(19) United States
(12) Patent Application Publication
(10) Pub. No.: US 2026/0364184 A1
(43) Pub. Date: Nov. 19, 2026

(54) COMPOUND BASED ON OXOISOINDOLINYL-SUBSTITUTED TETRAHYDROPYRIMIDINE-DIONE AND USE THEREOF

(71) Applicant: GLUETACS THERAPEUTICS (SHANGHAI) CO., LTD., Shanghai (CN)

(72) Inventors: Xiaobao Yang, Shanghai (CN); Yan Li, Shanghai (CN); Renhong Sun, Shanghai (CN); Baoyin Zhao, Shanghai (CN); Hailong Zhou, Shanghai (CN)

(73) Assignee: GLUETACS THERAPEUTICS (SHANGHAI) CO., LTD., Shanghai (CN)

(21) Appl. No.: 19/489,235

(22) PCT Filed: Jun. 3, 2024
(86) PCT No.: PCT/CN2024/097049

§ 371 Date: Dec. 1, 2025

(30) Foreign Application Priority Data
Jun. 1, 2023 (CN) 202310644215.6

(51) Int. Cl. A61K 47/55; A61K 47/54; A61P 37/02

(52) U.S. Cl. CPC A61K 47/55; A61K 47/545; A61P 37/02

(57) ABSTRACT

The present disclosure provides compounds of Formula (I) and (II) or salts, enantiomers, stereoisomers, solvates, isotopically enriched analogs, prodrugs, or polymorphs thereof, and uses thereof. The present disclosure also provides pharmaceutical compositions comprising, as an active ingredient, the compounds of Formula (I) or salts, enantiomers, stereoisomers, solvates, isotopically enriched analogs, prodrugs, or polymorphs thereof, and uses thereof.

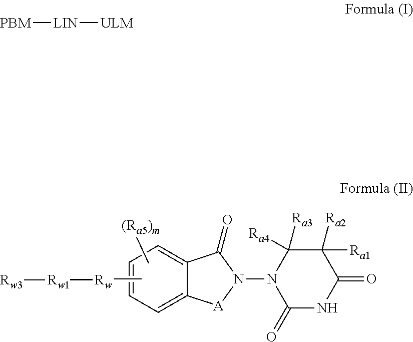

### TECHNICAL FIELD

The present disclosure relates to compounds of Formula (I), cereblon (CRBN) E3 ubiquitin ligase ligand compounds of Formula (II), and their applications. The CRBN E3 ubiquitin ligase ligand compounds of Formula (II) are capable of binding to the CRBN E3 ligase and are used in the preparation of the compounds of Formula (I). Based on the compounds of Formula (II) and various target protein ligands, the designed compounds of Formula (I) of the present disclosure can possess broad pharmacological activities through the degradation/inhibition of diverse pathogenic protein classes or families.

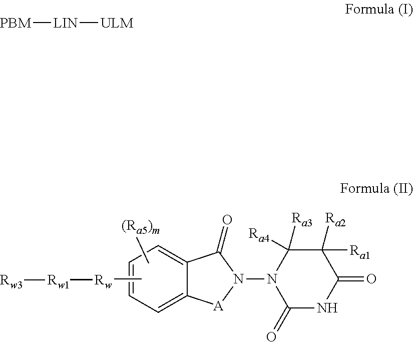

### BACKGROUND

Bifunctional proteolysis-targeting drugs consist of three moieties, one end of which is a ligand warhead that can bind to a specific target protein, and the other end is a small molecule ligand of E3 ubiquitin ligase, both of which are connected together through linkers of different lengths and categories located in the middle. The bifunctional proteolysis-targeting small molecules can utilize the binding effects of the ligands at both ends to simultaneously bind to a specific target protein and E3 ubiquitin ligase, thereby recruiting E3 ubiquitin ligase to the vicinity of the specific target protein and enabling E3 ubiquitin ligase to ubiquitinate the target protein. The polyubiquitinated target protein will be recognized and degraded by proteasomes in the body. The biggest difference between the bifunctional proteolysis-targeting drugs and traditional small molecule-based inhibitor drugs is that the former mobilize the entire cell as a drug effector unit. This drug action mode only requires small molecule drugs to briefly bind to the target protein and tag it for degradation. Therefore, a low drug dose can meet the requirements, greatly reduce the risk of off-target effects, and potentially eliminate tumor progression caused by abnormal expression of driver genes and drug resistance caused by acquired mutations in driver genes.

Over 600 distinct E3 ubiquitin ligases are involved in the design of proteolysis-targeting drugs; however, only a few have been successfully utilized in preclinical research on degraders. Among them, Cereblon (CRBN) E3 ubiquitin ligase is one of the most widely used E3 ubiquitin ligases. The known ligands for CRBN-type E3 ubiquitin ligase (CRBN ligands) include thalidomide and its analogs pomalidomide and lenalidomide, all of which possess a phthalimide backbone. Currently, several bifunctional protein degraders in clinical stages—such as ARV-110 targeting AR, ARV-471 targeting ER, NX-2127 targeting BTK, and FHD-609 targeting BRD9—are designed using CRBN ligands of the immunomodulatory imide drug (IMiD) type containing the phthalimide scaffold.

Upon binding to CRBN E3 ubiquitin ligase, IMiD-based CRBN ligands can themselves act as molecular glues, inducing CRBN to recognize novel substrate proteins, leading to their ubiquitination and degradation. This results in pharmacological effects such as antitumor and immunomodulatory activities. Molecular glue degraders directly target and eliminate specific proteins and offer potential advantages such as the ability to target “undruggable” proteins. Moreover, they typically exhibit low molecular weight and good drug-like properties, making them a major focus in drug development. In addition to the already marketed IMiD drugs, a range of molecular glue compounds based on the CRBN E3 ubiquitin ligase have been developed. Examples include CC-122, CC-220, CC-90009, CC-99282, and CC-92480 from Bristol Myers Squibb (BMS); DKY709 from Novartis; and CFT7455 from C4 Therapeutics, all of which are currently in clinical trials. The substrate spectrum of these molecular glues has also expanded from initially identified transcription factors IKZF1/3 to include casein kinase 1a (CK1a), zinc finger protein 91 (ZFP91), and translation termination factor GSPT1, among others. Degradation of these substrate proteins enables molecular glues to exert various pharmacological activities—such as immunomodulatory, anti-inflammatory, and antitumor effects—across different indications.

In summary, CRBN ligands can not only serve as components of bifunctional protein degraders to facilitate the effective degradation of specific disease-causing proteins, but can also function as molecular glue degraders inducing the degradation of diverse substrate proteins. Therefore, designing diverse molecular scaffolds and developing novel CRBN ligands with drug-like properties for application in bifunctional protein degrader development has become a major research focus in the field.

Despite this, such IMiD-based CRBN E3 ubiquitin ligase ligands with a phthalimide skeleton still face several challenges, notably including poor solubility and weak CRBN binding affinity. Furthermore, these IMID-based CRBN ligand compounds typically contain a piperidine-2,6-dione-substituted isoindolinone structure (i.e.,

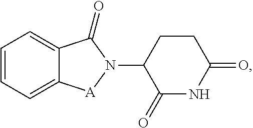

wherein A represents CO or CH₂). This structure incorporates a chiral center, resulting in the existence of R and S configurations. To obtain a single enantiomer, it is often necessary to employ costly methods such as asymmetric synthesis, enzymatic catalysis, or chiral resolution.

Consequently, there is a pressing need to develop a new series of novel CRBN ligands possessing high CRBN binding affinity and drug-like properties. These ligands are intended for the design and synthesis of corresponding bifunctional protein degraders targeting various proteins, for the treatment and/or prevention of target protein-mediated or associated diseases or disorders.

### SUMMARY OF INVENTION

In view of the above, an object of the present disclosure is to provide novel small molecule ligands for E3 ubiquitin ligases, bifunctional protein degraders designed based on such novel E3 ubiquitin ligase ligands and various target protein ligands, as well as their applications and usage methods. The E3 ligands provided herein exhibit stronger CRBN binding affinity and improved drug-like properties, and themselves demonstrate enhanced activity and/or selectivity as molecular glues for inducing substrate protein degradation. Furthermore, bifunctional protein degraders designed using the E3 ligands provided herein, targeting different proteins, show stronger degradation/inhibition effects against various classes or families of pathogenic proteins, favorable pharmacokinetic properties, and consequently elicit broad pharmacological activities.

To achieve the above objectives and other related purposes, in one aspect, the present disclosure provides novel CRBN E3 ubiquitin ligase ligands, which can function as molecular glues binding to the CRBN E3 ligase. The isoindolinyl-substituted tetrahydropyrimidinedione scaffold designed in the present invention addresses the chirality issue associated with the existing piperidine-2,6-dione-substituted isoindolinone structures. It exhibits CRBN binding affinity, favorable pharmacokinetic properties, and/or antitumor activity, thereby enabling its application in bifunctional protein-degrading small molecule drugs.

In some embodiments, the novel E3 ubiquitin ligase ligands of the present disclosure may be the compound of Formula (II):

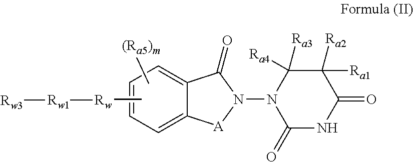

or salts, stereoisomers (including enantiomers), solvates, isotopically enriched analogs, prodrugs, or polymorphs thereof,
wherein A represents CO, CH₂, or CD₂;
Rₐ₁, Rₐ₂, Rₐ₃, and Rₐ₄ are the same or different and independently represent hydrogen, deuterium, halogen, C₁₋₆ alkyl, halogenated C₁₋₆ alkyl, deuterated C₁₋₆ alkyl, C₁₋₆ alkoxy, deuterated C₁₋₆ alkoxy, or halogenated C₁₋₆ alkoxy;
(Rₐ₅)ₘ indicates that the isoindoline ring to which it is attached is optionally substituted with m Ras, with each Ras being the same or different and each independently representing halogen, hydroxy, mercapto, nitro, amino, cyano, C₁₋₆ alkyl, halogenated C₁₋₆ alkyl, deuterated C₁₋₆ alkyl, C₁₋₆ alkoxy, deuterated C₁₋₆ alkoxy, halogenated C₁₋₆ alkoxy, C₂₋₆ alkenyl or C₂₋₆ alkynyl;
m represents an integer of 0, 1, 2, 3, or 4;
R_{w} represents O, S, S(O), S(O)₂, alkenylene, alkynylene, or N(Rₐ₆), wherein Rₐ₆ represents H or C₁₋₃ alkyl, or R_{w} represents a bond, or
R_{w} represents:

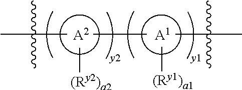

wherein each ring A¹ is the same or different and independently represents nitrogen-containing heterocyclylene, arylene, or heteroarylene, y1 represents an integer of 1 or 2, and (R_{y1})ₐ₁ indicates that each ring A¹ is independently optionally substituted with a1 R^{y1} substituents, wherein each R^{y1} independently represents deuterium, C₁₋₆ alkyl, deuterated C₁₋₆ alkyl, halogenated C₁₋₆ alkyl, C₁₋₆ alkoxy, halogen, amino, hydroxy, mercapto, cyano, carboxyl, C₂₋₆ alkynyl, or C₂₋₆ alkenyl, and a1 represents an integer of 0-20;
each ring A² is the same or different and independently represents heterocyclylene, cycloalkylene, arylene, or heteroarylene, y2 represents an integer of 0 or 1, and (R^{y2})ₐ₂ indicates that each ring A² is independently optionally substituted with a2 R^{y2} substituents, wherein each R^{y2} independently represents deuterium, C₁₋₆ alkyl, deuterated C₁₋₆ alkyl, halogenated C₁₋₆ alkyl, C₁₋₆ alkoxy, halogen, amino, hydroxy, mercapto, cyano, carboxyl, C₂₋₆ alkynyl, or C₂₋₆ alkenyl, and a2 represents an integer of 0-20; and
R_{w1} represents a bond or optionally substituted linear or branched C₁₋₂₀ alkylene, wherein any one or more methylene groups in the main carbon chain skeleton of the linear or branched C₁₋₂₀ alkylene are optionally replaced by one or more groups selected from Rₐ, R_{b}, and any combination thereof; wherein each Rₐ is independently selected from the group consisting of: O, C(O), OC(O), S, S(O), S(O)₂, S(O)₂N(Rₐ₇), N(Rₐ₇)S(O)₂, C(O)N(Rₐ₇), N(Rₐ₇)C(O), N(Rₐ₇), and N(Rₐ₇)C(O)N(Rₐ₇), where each Raz is independently H or C₁₋₃ alkyl, and when any one or more adjacent methylene in the main carbon chain skeleton of the linear or branched C₁₋₂₀ alkylene are replaced by one or more Rₐ groups, the Rₐ groups are not directly connected to each other; and wherein each R_{b} is independently selected from the group consisting of: optionally substituted cycloalkylene, optionally substituted arylene, optionally substituted heterocyclylene, optionally substituted heteroarylene, alkynylene, alkenylene, and any combination thereof, and
R_{w3} represents hydrogen, deuterium, leaving group, hydroxy, halogen, COOH, NH₂, N₃, CHO, methanesulfonyloxy, trifluoromethanesulfonyloxy, or p-toluenesulfonyloxy.

In another aspect, the bifunctional protein degraders designed based on the novel E3 ubiquitin ligase ligands and various target protein ligands may be the compound of Formula (I):

PBM-LIN-ULM Formula (I)

or salts, stereoisomers (including enantiomers), solvates, isotopically enriched analogs, prodrugs, or polymorphs thereof,
wherein PBM represents a target protein-binding moiety that is covalently linked to ULM via LIN;
wherein ULM represents an E3 ubiquitin ligase-binding ligand moiety, and represents the structure of Formula (ULM):

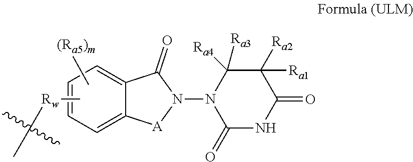

wherein A represents CO, CH₂, or CD₂;
Rₐ₁, Rₐ₂, Rₐ₃, and Rₐ₄ are the same or different and independently represent hydrogen, deuterium, halogen, C₁₋₆ alkyl, halogenated C₁₋₆ alkyl, deuterated C₁₋₆ alkyl, C₁₋₆ alkoxy, deuterated C₁₋₆ alkoxy, or halogenated C₁₋₆ alkoxy;
(Rₐ₅)ₘ indicates that the isoindoline ring to which it is attached is optionally substituted with m Ras, with each Ras being the same or different and each independently representing halogen, hydroxy, mercapto, nitro, amino, cyano, C₁₋₆ alkyl, halogenated C₁₋₆ alkyl, deuterated C₁₋₆ alkyl, C₁₋₆ alkoxy, deuterated C₁₋₆ alkoxy, halogenated C₁₋₆ alkoxy, C₂₋₆ alkenyl or C₂₋₆ alkynyl;
m represents an integer of 0, 1, 2, 3, or 4;
R_{w} represents O, S, S(O), S(O)₂, alkenylene, alkynylene, or N(Rₐ₆), wherein Rₐ₆ represents H or C₁₋₃ alkyl, or R_{w} represents a bond, or
R_{w} represents:

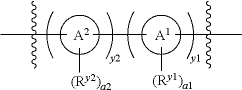

wherein each ring A¹ is the same or different and independently represents nitrogen-containing heterocyclylene, arylene, or heteroarylene, y1 represents an integer of 1 or 2, and (R_{y1})ₐ₁ indicates that each ring A¹ is independently optionally substituted with a1 R^{y1} substituents, wherein each R^{y1} independently represents deuterium, C₁₋₆ alkyl, deuterated C₁₋₆ alkyl, halogenated C₁₋₆ alkyl, C₁₋₆ alkoxy, halogen, amino, hydroxy, mercapto, cyano, carboxyl, C₂₋₆ alkynyl, or C₂₋₆ alkenyl, and a1 represents an integer of 0-20;
each ring A² is the same or different and independently represents heterocyclylene, cycloalkylene, arylene, or heteroarylene, y2 represents an integer of 0 or 1, and (R^{y2})ₐ₂ indicates that each ring A² is independently optionally substituted with a2 R^{y2} substituents, wherein each R^{y2} independently represents deuterium, C₁₋₆ alkyl, deuterated C₁₋₆ alkyl, halogenated C₁₋₆ alkyl, C₁₋₆ alkoxy, halogen, amino, hydroxy, mercapto, cyano, carboxyl, C₂₋₆ alkynyl, or C₂₋₆ alkenyl, and a2 represents an integer of 0-20; and LIN represents:

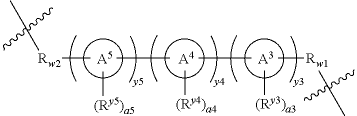

wherein R_{w2} is linked to PBM, and R_{w1} is linked to R_{w};
R_{w2} represents a bond, C(O), C(O)NH, or NHC(O);
each ring A³ is the same or different and independently represents heterocyclylene, cycloalkylene, arylene, or heteroarylene, y3 represents an integer of 0, 1, 2, or 3, and (R^{y3})ₐ₃ indicates that each ring A³ is independently optionally substituted with a3 R^{y3} substituents, wherein each R^{y3} independently represents deuterium, C₁₋₆ alkyl, deuterated C₁₋₆ alkyl, halogenated C₁₋₆ alkyl, C₁₋₆ alkoxy, halogen, amino, hydroxy, mercapto, cyano, carboxyl, C₂₋₆ alkynyl, or C₂₋₆ alkenyl, and a3 represents an integer of 0-10;
each ring A⁴ is the same or different and independently represents heterocyclylene, cycloalkylene, arylene, or heteroarylene, y4 represents an integer of 0, 1, 2, or 3, and (R^{y4})ₐ₄ indicates that each ring A⁴ is independently optionally substituted with a4 R^{y4} substituents, wherein each R^{y4} independently represents deuterium, C₁₋₆ alkyl, deuterated C₁₋₆ alkyl, halogenated C₁₋₆ alkyl, C₁₋₆ alkoxy, halogen, amino, hydroxy, mercapto, cyano, carboxyl, C₂₋₆ alkynyl, or C₂₋₆ alkenyl, and a4 represents an integer of 0-10;
each ring A⁵ is the same or different and independently represents heterocyclylene, cycloalkylene, arylene, or heteroarylene, y5 represents an integer of 0, 1, 2, or 3, and (R^{y5}) as indicates that each ring A⁵ is independently optionally substituted with a5 R^{y5} substituents, with each R^{y5} independently representing deuterium, C₁₋₆ alkyl, deuterated C₁₋₆ alkyl, halogenated C₁₋₆ alkyl, C₁₋₆ alkoxy, halogen, amino, hydroxy, mercapto, cyano, carboxyl, C₂₋₆ alkynyl, or C₂₋₆ alkenyl, and a5 represents an integer of 0-10;
R_{w1} represents a bond or optionally substituted linear or branched C₁₋₂₀ alkylene, wherein any one or more methylene groups in the main carbon chain skeleton of the linear or branched C₁₋₂₀ alkylene are optionally replaced by one or more groups selected from Rₐ, R_{b}, and any combination thereof; wherein each Rₐ is independently selected from the group consisting of: O, C(O), OC(O), S, S(O), S(O)₂, S(O)₂N(Rₐ₇), N(Rₐ₇)S(O)₂, C(O)N(Rₐ₇), N(Rₐ₇)C(O), N(Rₐ₇), and N(Rₐ₇)C(O)N(Rₐ₇), where each Rₐ₇ is independently H or C₁₋₃ alkyl, and when one or more adjacent methylene in the main carbon chain skeleton of the linear or branched C₁₋₂₀ alkylene are replaced by one or more Rₐ groups, the Rₐ groups are not directly connected to each other; and wherein each R_{b} is independently selected from the group consisting of: optionally substituted cycloalkylene, optionally substituted arylene, optionally substituted heterocyclylene, optionally substituted heteroarylene, alkynylene, alkenylene, and any combination thereof.

In a further aspect, the present disclosure provides a pharmaceutical composition comprising the compound of Formula (I) or salts, stereoisomers (including enantiomers), solvates, isotopically enriched analogs, prodrugs, or polymorphs thereof, and at least one pharmaceutically acceptable carrier.

In a further aspect, the present disclosure further provides a medicine kit or reagent kit comprising: the compound of Formula (I) or a pharmaceutically acceptable salt, or the pharmaceutical composition comprising the same.

In a further aspect, the present disclosure provides the compound of Formula (I) or salts, stereoisomers (including enantiomers), solvates, isotopically enriched analogs, prodrugs, or polymorphs thereof, or the pharmaceutical composition comprising the same as active ingredient, for use as a medicament.

In a further aspect, the present disclosure provides the compound of Formula (I) or salts, stereoisomers (including enantiomers), solvates, isotopically enriched analogs, prodrugs, or polymorphs thereof, or the pharmaceutical composition comprising the same as active ingredient for use in the treatment and/or prevention of a disease or disorder selected from the group consisting of tumors, autoinflammatory diseases, inflammatory diseases, autoimmune diseases, neurological disorders, respiratory diseases, hemorrhagic shock, transplant rejection, multiple organ dysfunction syndrome (MODS), sarcoidosis, adult respiratory distress syndrome, cardiovascular diseases, Richter syndrome (RS), acute liver failure, diabetes, Kennedy disease, seborrheic alopecia, hirsutism, skin diseases, dysfunctional uterine bleeding, anemia, pediatric aplastic anemia, endometriosis, polycystic ovary syndrome, and thyroid diseases.

In a further aspect, the present disclosure provides the use of the compound of Formula (I) or salts, stereoisomers (including enantiomers), solvates, isotopically enriched analogs, prodrugs, or polymorphs thereof, or the pharmaceutical composition as described herein, for the manufacture of a medicament for the prevention or treatment of a disease or disorder selected from the group consisting of tumors, autoinflammatory diseases, inflammatory diseases, autoimmune diseases, neurological disorders, respiratory diseases, hemorrhagic shock, transplant rejection, multiple organ dysfunction syndrome (MODS), sarcoidosis, adult respiratory distress syndrome, cardiovascular diseases, Richter syndrome (RS), acute liver failure, diabetes, Kennedy disease, seborrheic alopecia, hirsutism, skin diseases, dysfunctional uterine bleeding, anemia, pediatric aplastic anemia, endometriosis, polycystic ovary syndrome, and thyroid diseases.

In a further aspect, the present disclosure provides a method for treating or preventing a disease or disorder in a subject, comprising administering to the subject a therapeutically effective amount of the compound of Formula (I) or salts, enantiomers, stereoisomers, solvates, isotopically enriched analogs, prodrugs, or polymorphs thereof, or the pharmaceutical composition comprising the same, wherein the disease or disorder comprises tumors, autoinflammatory diseases, inflammatory diseases, autoimmune diseases, neurological disorders, respiratory diseases, hemorrhagic shock, transplant rejection, multiple organ dysfunction syndrome (MODS), sarcoidosis, adult respiratory distress syndrome, cardiovascular diseases, Richter syndrome (RS), acute liver failure, diabetes, Kennedy disease, seborrheic alopecia, hirsutism, skin diseases, dysfunctional uterine bleeding, anemia, pediatric aplastic anemia, endometriosis, polycystic ovary syndrome, and thyroid diseases.

In a further aspect, the present disclosure also provides a use of the compound of Formula (II) or salts, stereoisomers (including enantiomers), solvates, isotopically enriched analogs, prodrugs, or polymorphs thereof for the preparation of the compound of Formula (I) or salts, enantiomers, stereoisomers, solvates, isotopically enriched analogs, prodrugs, or polymorphs thereof of the present disclosure.

In a further aspect, the present disclosure also provides a method for preparing the compound of Formula (I) or salts, enantiomers, stereoisomers, solvates, isotopically enriched analogs, prodrugs, or polymorphs thereof of the present disclosure by using the compound of Formula (II) or salts, stereoisomers (including enantiomers), solvates, isotopically enriched analogs, prodrugs, or polymorphs thereof.

In a further aspect, the present disclosure provides a pharmaceutical composition comprising the compound of Formula (II) or salts, stereoisomers (including enantiomers), solvates, isotopically enriched analogs, prodrugs, or polymorphs thereof, and at least one pharmaceutically acceptable carrier.

In a further aspect, the present disclosure further provides a medicine kit or reagent kit comprising: the compound of Formula (II) or a pharmaceutically acceptable salt, or the pharmaceutical composition comprising the same.

In a further aspect, the present disclosure provides the compound of Formula (II) or salts, stereoisomers (including enantiomers), solvates, isotopically enriched analogs, prodrugs, or polymorphs thereof, or the pharmaceutical composition comprising the same as active ingredient, for use as a medicament.

In a further aspect, the present disclosure provides the compound of Formula (II) or salts, stereoisomers (including enantiomers), solvates, isotopically enriched analogs, prodrugs, or polymorphs thereof, or the pharmaceutical composition comprising the same as active ingredient for use in the prevention and/or treatment of a disease or disorder associated with a cereblon protein.

In a further aspect, the present disclosure provides the use of the compound of Formula (II) or pharmaceutically acceptable salts, stereoisomers (including enantiomers, diastereomers), solvates, isotopically enriched analogs, prodrugs, or polymorphs thereof, or the pharmaceutical composition as described herein, for the manufacture of a medicament for the prevention or treatment of a disease or disorder associated with a cereblon protein.

In a further aspect, the present disclosure provides a method for treating or preventing a disease or disorder associated with a cereblon protein in a subject, comprising administering to the subject a therapeutically effective amount of the compound of Formula (II) or salts, enantiomers, stereoisomers, solvates, isotopically enriched analogs, prodrugs, or polymorphs thereof, or the pharmaceutical composition comprising the same.

The disease or disorder associated with cereblon protein comprises tumors, infectious diseases, inflammatory diseases, autoimmune diseases, anemia, hemorrhagic shock, transplant rejection, multiple organ dysfunction syndrome (MODS), sarcoidosis, adult respiratory distress syndrome, cardiovascular diseases, Richter syndrome (RS), acute liver failure, or diabetes.

### DESCRIPTION OF DRAWINGS

FIG. 1 shows the Western blot experimental results of the degradation of the target substrate protein by the compounds of the present disclosure in cells.

### DETAILED DESCRIPTION OF THE INVENTION

The following detailed description is provided as exemplary specific embodiments to assist those skilled in the art in understanding and practicing the present disclosure. It should be appreciated, however, that such description is not intended to limit the scope of the present disclosure, and that various modifications and changes may be made to the specific embodiments described in the present disclosure without departing from the spirit and scope of the present disclosure. Such changes and modifications are to be understood as being included within the scope of the present invention as defined by the appended claims.

### I. Compounds

### Compounds of Formula (I)

The compound of Formula (I) of the present disclosure, or salts (including pharmaceutically acceptable salts), stereoisomers (including enantiomers), solvates, isotopically enriched analogs, prodrugs, or polymorphs thereof, can bind to the target protein, recruit the target protein to an E3 ubiquitin ligase for ubiquitination, and subsequently lead to its degradation. The compound of Formula (I) of the present disclosure can specifically target particular classes or families of target proteins, and exhibit a broad spectrum of pharmacological activities by degrading/inhibiting diverse classes or families of target proteins.

PBM-LIN-ULM Formula (I)

wherein PBM, LIN, and ULM are as defined in the compounds of Formula (I) above.

In some embodiments, the PBM may be a small molecule ligand that binds to specific target proteins, including but not limited to: epidermal growth factor receptor (EGFR); Cyclin-dependent kinase 4/6 (CDK4/6); anaplastic lymphoma kinase (ALK); activin receptor-like kinase 2 (ALK2); fibroblast growth factor receptors (FGFRs); proto-oncogene tyrosine-protein kinase receptor RET (RET); Focal adhesion kinase (FAK); breakpoint cluster region (BCR)-Abelson leukemia virus (ABL)(BCR-ABL) tyrosine kinase; Bruton tyrosine kinase (BTK); Androgen receptor (AR); Estrogen receptor (ER); Bromodomain and extra-terminal domain protein (BET); Interleukin-1 receptor-associated kinase 4 (IRAK4); Cyclin-dependent kinase 9 (CDK9); Cyclin-dependent kinase 2 (CDK2); serine/threonine protein kinase (AKT), microtubule-associated protein tau; Histone-lysine N-methyltransferase EZH2 (EZH2); neurotrophic receptor tyrosine kinase (NTRK); Src homology 2 domain containing protein tyrosine phosphatase (SHP2); Poly (ADP-ribose) polymerase (PARP); signal transducers and activators of transcription 3 (STAT3); FMS-like tyrosine kinase 3 (FLT3); B cell lymphoma-2 (BCL-2) family proteins; SOS Ras/Rac guanine nucleotide exchange factor 1 (SOS1); GTPase Kras (KRAS); SWI/SNF related, matrix associated, actin dependent regulator of chromatin, subfamily a, member 2/4 (SMARCA2/4); and ribosomal protein S6 kinase (RSK).

In some embodiments, PBM comprises the structures of the following formula:

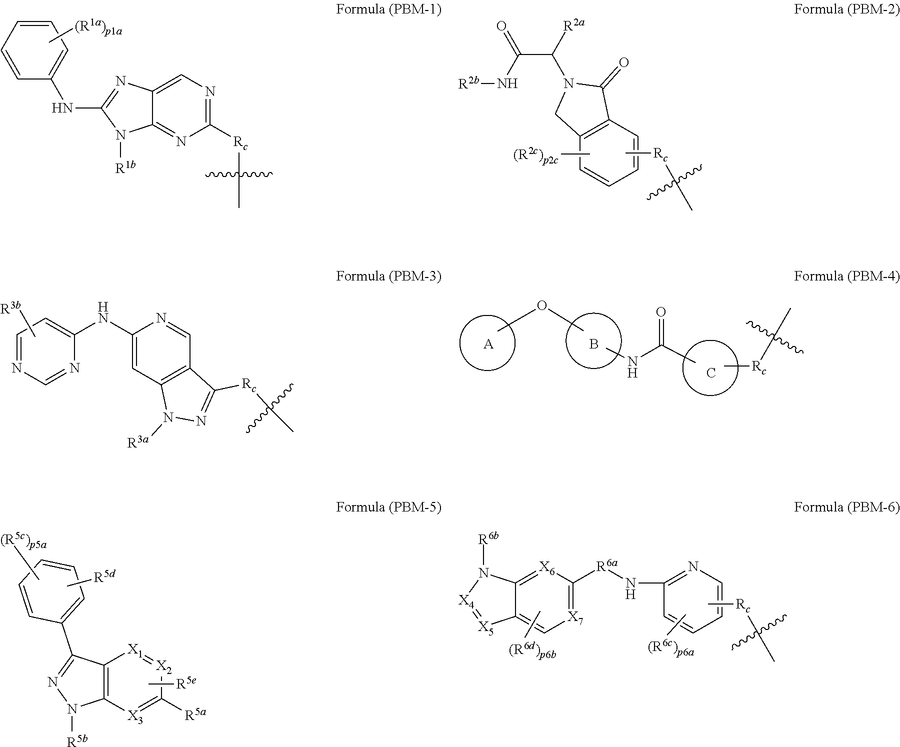

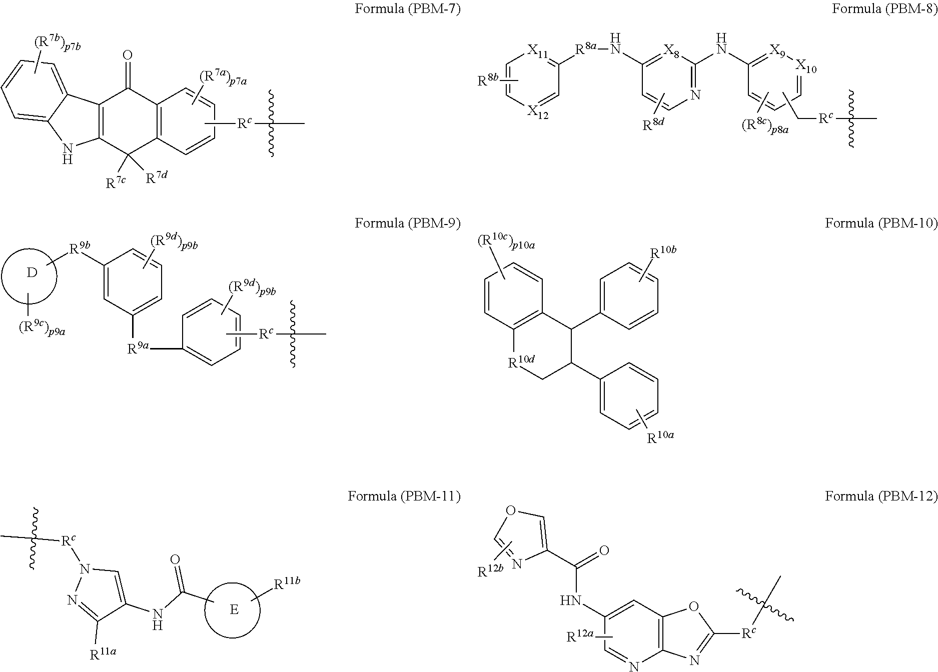

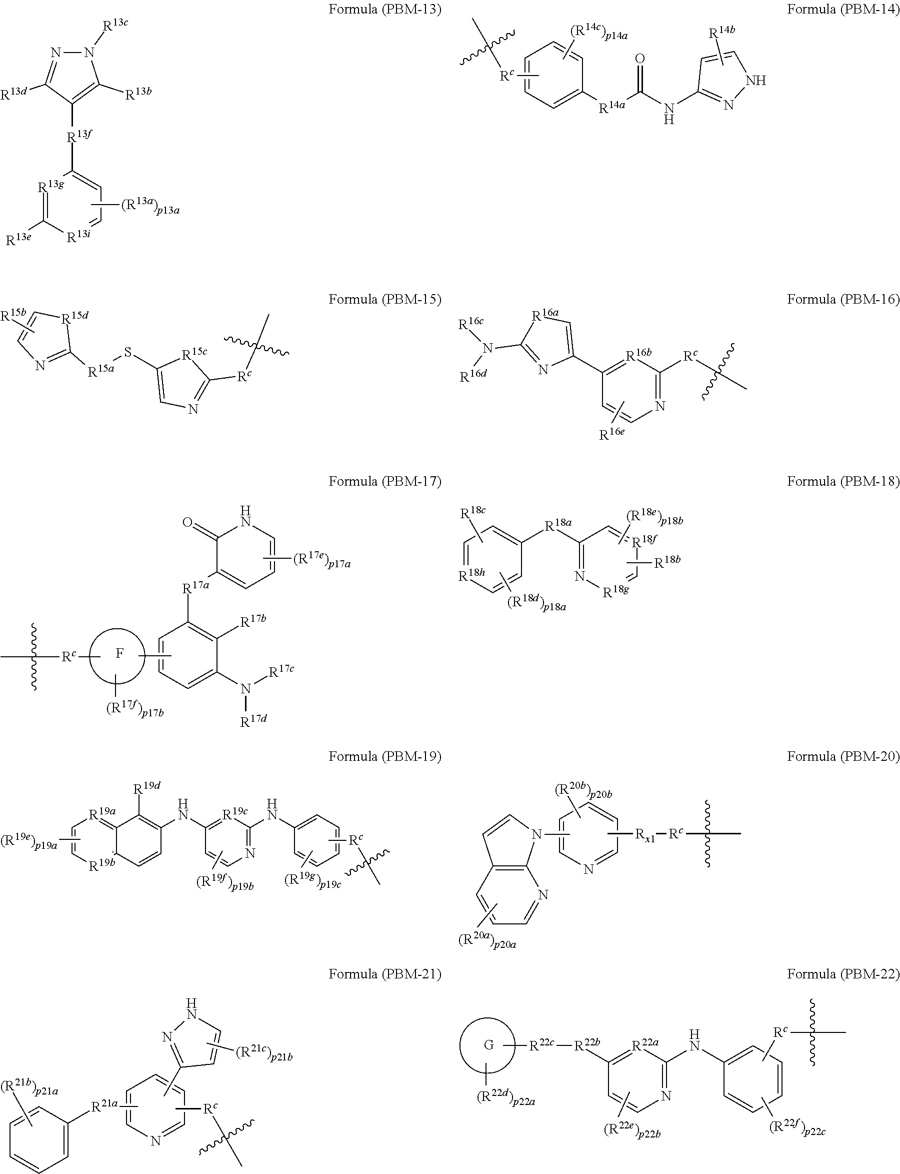

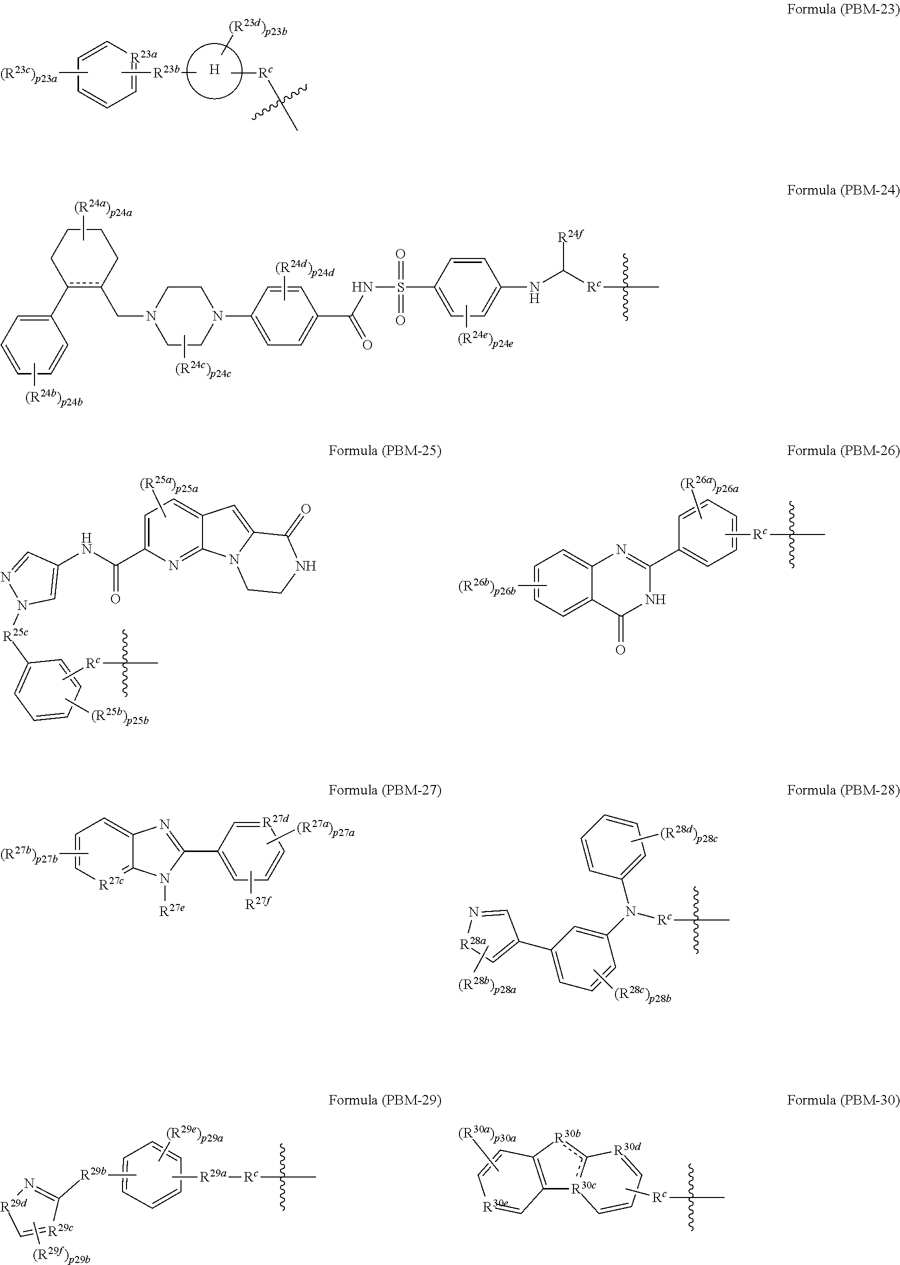

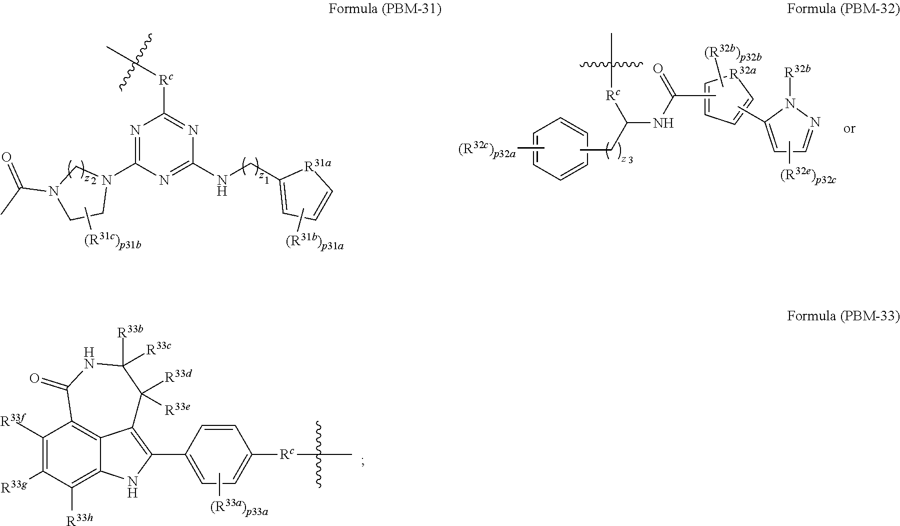

wherein
(R^{1a})ₚ₁ₐ in Formula (PBM-1) indicates that the benzene ring to which it is attached is substituted with p1a R^{1a} groups, with each R^{1a} being the same or different and independently representing deuterium, halogen, hydroxy, C₁₋₆ alkyl, deuterated C₁₋₆ alkyl, halogenated C₁₋₆ alkyl, C₁₋₆ alkoxy, halogenated C₁₋₆ alkoxy, NO₂, NH₂, or cyano;
p1a represents an integer of 0, 1, 2, 3, 4, or 5;
R^{1b} represents substituted or unsubstituted C₃₋₁₅ cycloalkyl, such as cyclopentyl;
R^{2a} in Formula (PBM-2) represents C₆₋₁₀ aryl or 5-to 20-membered heteroaryl, where the C₆₋₁₀ aryl and 5-to 20-membered heteroaryl are each independently optionally substituted with one or more (e.g., 1-10, 1-8, 2-6, 1-5, 1-4, 1-3, or 2-5) substituents independently selected from the group consisting of deuterium, halogen, hydroxy, cyano, amino, nitro, C₁₋₆ alkyl, halogenated C₁₋₆ alkyl, deuterated C₁₋₆ alkyl, C₁₋₆ alkoxy, halogenated C₁₋₆ alkoxy, and any combination thereof;
R^{2b} represents C₆₋₁₀ aryl or 5-to 20-membered heteroaryl (e.g., 5-to 20-membered heteroaryl containing one or two 5-to 7-membered rings and 1 to 4 heteroatoms selected from N, O, and S), wherein the C₆₋₁₀ aryl and the 5-to 20-membered heteroaryl are each independently optionally substituted with one or more (e.g., 1-10, 1-8, 2-6, 1-5, 1-4, 1-3, or 2-5) R^{2d}, with each R^{2d} being the same or different and each independently representing deuterium, halogen, hydroxy, cyano, amino, nitro, C₁₋₆ alkyl, halogenated C₁₋₆ alkyl, deuterated C₁₋₆ alkyl, C₁₋₆ alkoxy, or halogenated C₁₋₆ alkoxy;
(R^{2c})ₚ₂ₐ indicates that the isoindoline ring in Formula (PBM-2) is optionally substituted with p2a R^{2c}, with each R^{2c} being the same or different and independently representing deuterium, halogen, hydroxy, C₁₋₆ alkyl, halogenated C₁₋₆ alkyl, deuterated C₁₋₆ alkyl, NO₂, NH₂, C₁₋₆ alkoxy, halogenated C₁₋₆ alkoxy, or cyano;
p2a represents an integer of 0, 1, 2, or 3;
R^{3a} in Formula (PBM-3) represents hydrogen, C₁₋₁₀ alkyl

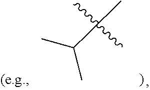 [SMILES: *C(C)C]

deuterated C₁₋₁₀ alkyl, or halogenated C₁₋₁₀ alkyl;
R^{3b} represents

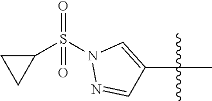 [SMILES: *c1cnn(S(=O)(=O)C2CC2)c1]

or 4-to 12-membered nitrogen-containing heterocyclyl, wherein the 4-to 12-membered nitrogen-containing heterocyclyl is optionally substituted with one or more (e.g., 1-10, 1-8, 2-6, 1-5, 1-4, 1-3, or 2-5) substituents independently selected from the group consisting of deuterium, halogen, hydroxy, C₁₋₆ alkyl, halogenated C₁₋₆ alkyl, deuterated C₁₋₆ alkyl, C₁₋₆ alkoxy, halogenated C₁₋₆ alkoxy, deuterated C₁₋₆ alkoxy, NO₂, NH₂, cyano, and any combination thereof;
ring A in Formula (PBM-4) represents C₆₋₁₀ aryl or 5-to 20-membered heteroaryl (e.g., 5-to 20-membered heteroaryl containing one or two 5-to 7-membered rings and 1 to 4 heteroatoms selected from N, O, and S), and the ring A is optionally substituted with one or more (e.g., 1-10, 1-8, 2-6, 1-5, 1-4, 1-3, or 2-5) R^{4a}, with each R^{4a} being the same or different and each independently representing deuterium, halogen, hydroxy, cyano, amino, nitro, C₁₋₆ alkyl, halogenated C₁₋₆ alkyl, deuterated C₁₋₆ alkyl, C₁₋₆ alkoxy, halogenated C₁₋₆ alkoxy, C₂₋₆ alkenyl or C₂₋₆ alkynyl;
ring B in Formula (PBM-4) represents C₃₋₁₅ cycloalkyl or 4-to 20-membered heterocyclyl (e.g., 4-to 20-membered heterocyclyl containing 1 to 4 heteroatoms selected from N, O and S), wherein the ring B is optionally substituted with one or more (e.g., 1-10, 1-8, 2-6, 1-5, 1-4, 1-3, or 2-5) R^{4b}, with each R^{4b} being the same or different and each independently representing deuterium, halogen, C₁₋₆ alkyl, halogenated C₁₋₆ alkyl, deuterated C₁₋₆ alkyl, C₁₋₆ alkoxy, or halogenated C₁₋₆ alkoxy;
ring C in Formula (PBM-4) represents 5-to 20-membered heteroaryl (e.g., 5-to 20-membered monocyclic or bicyclic heteroaryl containing 1 to 4 heteroatoms selected from N, O and S), wherein the ring C is optionally substituted with one or more (e.g., 1-10, 1-8, 2-6, 1-5, 1-4, 1-3, or 2-5) R^{4c}, with each R^{4c} being the same or different and each independently representing deuterium, halogen, hydroxy, cyano, amino, nitro, C₁₋₆ alkyl, halogenated C₁₋₆ alkyl, deuterated C₁₋₆ alkyl, C₁₋₆ alkoxy, or halogenated C₁₋₆ alkoxy;
X₁ in Formula (PBM-5) represents N or CR^{5f}, where R^{5f} represents hydrogen, deuterium, or amino, and X₂ represents N or CR^{5g}, where R^{5g} represents hydrogen or R_{c}, and X₃ represents CH or N;
R^{5a} and R^{5b} each independently represent hydrogen or R_{c};
(R^{5c})ₚ₅ₐ in Formula (PBM-5) indicates that the benzene ring to which it is attached is substituted with p5a R^{5c}, with each R^{5c} being the same or different and independently representing —O-aryl, —O-heteroaryl, 4-to 20-membered heterocyclyl, —C₁₋₃ alkylene-NHC(O)-heteroaryl, —C₁₋₃ alkylene-C(O)NH-heteroaryl, C₁₋₆ alkyl, deuterium, deuterated C₁₋₆ alkyl, halogen, or halogenated C₁₋₆ alkyl, wherein the 4-to 20-membered heterocyclyl, aryl, and heteroaryl are each independently optionally substituted with one or more (e.g., 1-10, 1-8, 2-6, 1-5, 1-4, 1-3, or 2-5) R^{5h}, with each R^{5h} being the same or different and each independently representing deuterium, halogen, hydroxy, cyano, amino, C₁₋₆ alkyl, halogenated C₁₋₆ alkyl, deuterated C₁₋₆ alkyl, C₁₋₆ alkoxy, or halogenated C₁₋₆ alkoxy;
p1a represents an integer of 1, 2, 3, or 4;
R^{5d} represents R_{c}, hydrogen, halogen, C₁₋₆ alkyl, halogenated C₁₋₆ alkyl, deuterated C₁₋₆ alkyl, halogenated C₁₋₆ alkoxy, or C₁₋₆ alkoxy;
R^{5e} represents hydrogen, halogen, cyano, C₁₋₆ alkyl, halogenated C₁₋₆ alkyl, or amino;
wherein R^{5g}, R^{5a}, R^{5b}, and R^{5d} are not simultaneously hydrogen, and only one of R^{5g}, R^{5a}, R^{5b}, and R^{5d} represents R_{c}, wherein
   when R^{5a} represents R_{c}, X₂ represents CH or N, and R^{5b} is hydrogen, and R^{5d} represents hydrogen, halogen, C₁₋₆ alkyl, halogenated C₁₋₆ alkyl, deuterated C₁₋₆ alkyl, halogenated C₁₋₆ alkoxy, or C₁₋₆ alkoxy;
   when X₂ represents CR^{5g}, where R^{5g} represents R_{c}, R^{5a} and R^{5b} are hydrogen, and R^{5d} represents hydrogen, halogen, C₁₋₆ alkyl, halogenated C₁₋₆ alkyl, deuterated C₁₋₆ alkyl, halogenated C₁₋₆ alkoxy, or C₁₋₆ alkoxy;
   when R^{5b} represents R_{c}, X₂ represents N or CH, and R^{5a} is hydrogen, and R^{5d} represents hydrogen, halogen, C₁₋₆ alkyl, halogenated C₁₋₆ alkyl, deuterated C₁₋₆ alkyl, halogenated C₁₋₆ alkoxy, or C₁₋₆ alkoxy, and
   when R^{5d} represents R_{c}, X₂ represents N or CH, and R^{5a} and R^{5b} are hydrogen;
X₄ in Formula (PBM-6) represents CR^{6e} or a fragment

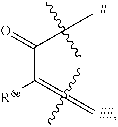

symbol #indicates the point of attachment to N atom adjacent to X₄, symbol ##indicates the point of attachment to X₅, and each R^{6e} independently represents deuterium, hydrogen, halogen, C₁₋₆ alkyl, halogenated C₁₋₆ alkyl, deuterated C₁₋₆ alkyl, —C(O)—C₁₋₆ alkyl, —C(O)-deuterated C₁₋₆ alkyl, or —C(O)NR^{6f}R^{6g}, where R^{6f} and R^{6g} are the same or different and independently represent hydrogen, C₁₋₆ alkyl, or deuterated C₁₋₆ alkyl;
X₅ in Formula (PBM-6) represents N or CR^{6h}, where R^{6h} represents hydrogen, deuterium, halogen, C₁₋₆ alkyl, deuterated C₁₋₆ alkyl, or halogenated C₁₋₆ alkyl;
X₆ and X₇ each independently represent CH or N;
R^{6a} represents a bond or optionally substituted heteroarylene (e.g., heteroarylene substituted with halogen or deuterium);
R^{6b} represents hydrogen, C₁₋₆ alkyl, halogenated C₁₋₆ alkyl, deuterated C₁₋₆ alkyl, or optionally substituted C₃₋₁₂ cycloalkyl;
(R^{6c})ₚ₆ₐ in Formula (PBM-6) indicates that the pyridine ring to which it is attached is optionally substituted with p6a R^{6c}, with each R^{6c} being the same or different and each independently representing deuterium, halogen, C₁₋₆ alkyl, C₁₋₆ alkoxy, halogenated C₁₋₆ alkoxy, deuterated C₁₋₆ alkyl, or halogenated C₁₋₆ alkyl, and p6a represents an integer of 0, 1, 2, or 3;
(R^{6d})_{p6b} in Formula (PBM-6) indicates that the nitrogen-containing bicyclic heteroaryl ring to which it is attached is optionally substituted with p6b R^{6d}, with each Rod being the same or different and each independently representing deuterium, halogen, C₁₋₆ alkyl, C₁₋₆ alkoxy, halogenated C₁₋₆ alkoxy, deuterated C₁₋₆ alkyl, or halogenated C₁₋₆ alkyl, and p6b represents an integer of 0 or 1;
(R^{7a})ₚ₇ₐ in Formula (PBM-7) indicates that the benzene ring to which it is attached is optionally substituted with p7a R^{7a}, with each R^{7a} being the same or different and each independently representing deuterium, cyano, halogen, C₁₋₆ alkyl, C₁₋₆ alkoxy, halogenated C₁₋₆ alkoxy, halogenated C₁₋₆ alkyl, or deuterated C₁₋₆ alkyl, and p7a represents an integer of 0, 1, 2, or 3;
(R^{7b})_{p7b} indicates that the benzene ring to which it is attached is optionally substituted with p7b R^{7b}, with each R^{7b} being the same or different and each independently representing deuterium, cyano, halogen, C₁₋₆ alkyl, C₁₋₆ alkoxy, halogenated C₁₋₆ alkoxy, halogenated C₁₋₆ alkyl, or deuterated C₁₋₆ alkyl, and p7b represents an integer of 0, 1, 2, 3, or 4;
R^{7c} and R^{7d} are the same or different and each independently represent deuterium, C₁₋₆ alkyl, halogen, halogenated C₁₋₆ alkyl, deuterated C₁₋₆ alkyl, C₁₋₆ alkoxy, or halogenated C₁₋₆ alkoxy;
X₈, X₉, X₁₀, X₁₁, and X₁₂ in Formula (PBM-8) are the same or different and independently represent N or CH;
R^{8a} represents a bond or optionally substituted methylene (e.g., halogenated methylene);
R^{8b} represents —C(O)N(R^{8f})R^{8e}, —N(R^{8f})C(O)R^{8e}, —S(O)₂N(R^{8f})R^{8a}, —N(R^{8f})S(O)₂R^{8e}, —S(O)₂R^{8e}, or —P(O)(R^{8e})₂, wherein each R^{8e} is the same or different and each independently represents C₁₋₆ alkyl or deuterated C₁₋₆ alkyl, and R^{8f} represents hydrogen or C₁₋₆ alkyl;
(R^{8c})ₚ₈ₐ in Formula (PBM-8) indicates that the 6-membered ring containing X₉ and X₁₀ to which it is attached is optionally substituted with p8a R^{8c}, with each R^{8c} being the same or different and each independently representing deuterium, halogen, hydroxy, amino, cyano, C₁₋₆ alkoxy, halogenated C₁₋₆ alkoxy, C₁₋₆ alkyl, deuterated C₁₋₆ alkyl, or halogenated C₁₋₆ alkyl;
p8a represents an integer of 0, 1, 2, 3, or 4;
R^{8d} represents deuterium, halogen, hydroxy, amino, cyano, C₁₋₆ alkoxy, halogenated C₁₋₆ alkoxy, C₁₋₆ alkyl, deuterated C₁₋₆ alkyl, or halogenated C₁₋₆ alkyl;
R^{9a} in Formula (PBM-9) represents —NHC(O)— or —C(O)NH—;
R^{9b} represents —NH—, —N(C₁₋₆ alkyl)-, or ethynylene;
ring D in Formula (PBM-9) represents a 5-to 20-membered monocyclic, bicyclic, or polycyclic heteroaryl group containing 1 to 4 heteroatoms selected from N, O, and S, and (R^{pc})ₚ₉ₐ indicates that the ring D is optionally substituted with p9a R^{9c}, with each R^{9c} being the same or different and each independently representing optionally substituted 5-to 15-membered heteroaryl, deuterium, halogen, hydroxy, cyano, amino, C₁₋₆ alkyl, halogenated C₁₋₆ alkyl, deuterated C₁₋₆ alkyl, C₁₋₆ alkoxy, or halogenated C₁₋₆ alkoxy;
p9a represents an integer of 0, 1, 2, or 3;
each (R^{9d})_{p9b} in Formula (PBM-9) independently indicates that the benzene ring to which it is attached is independently optionally substituted with p9b R^{9d}, with each Rod being the same or different and each independently representing deuterium, halogen, hydroxy, cyano, amino, C₁₋₆ alkoxy, halogenated C₁₋₆ alkoxy, C₁₋₆ alkyl, deuterated C₁₋₆ alkyl, or halogenated C₁₋₆ alkyl;
each p9b is the same or different and independently represents an integer of 0, 1, 2, 3, or 4;
R^{10d} in Formula (PBM-10) represents O, S, or CH₂;
(R^{10c})ₚ₁₀ₐ indicates that the benzo-fused six-membered ring containing R^{10d} in Formula (PBM-10) is optionally substituted with p10a R^{10c}, with each R^{10c} being the same or different and independently representing deuterium, halogen, hydroxy, cyano, amino, C₁₋₆ alkoxy, halogenated C₁₋₆ alkoxy, C₁₋₆ alkyl, deuterated C₁₋₆ alkyl, or halogenated C₁₋₆ alkyl;
p10a represents an integer of 0, 1, 2, 3, or 4;
only one of R^{10a} and R^{10b} represents R_{c}, and the other represents hydrogen, halogen, hydroxy, cyano, amino, C₁₋₆ alkoxy, halogenated C₁₋₆ alkoxy, C₁₋₆ alkyl, deuterated C₁₋₆ alkyl, or halogenated C₁₋₆ alkyl;
ring E in Formula (PBM-11) represents a 5-to 20-membered monocyclic, bicyclic, or polycyclic heteroarylene group containing 1 to 4 heteroatoms selected from N, O, and S;
R^{11b} represents optionally substituted 5-to 15-membered heteroaryl, optionally substituted 5-to 15-membered heterocyclyl, deuterium, halogen, hydroxy, cyano, amino, C₁₋₆ alkyl, halogenated C₁₋₆ alkyl, deuterated C₁₋₆ alkyl, C₁₋₆ alkoxy, or halogenated C₁₋₆ alkoxy;
R^{11a} represents hydrogen, halogen, C₁₋₆ alkyl, halogenated C₁₋₆ alkyl, deuterated C₁₋₆ alkyl, C₁₋₆ alkoxy, or halogenated C₁₋₆ alkoxy;
R^{12a} in Formula (PBM-12) represents optionally substituted 5-to 15-membered heterocyclyl, optionally substituted 5-to 15-membered heteroaryl, deuterium, halogen, hydroxy, cyano, amino, C₁₋₆ alkyl, halogenated C₁₋₆ alkyl, deuterated C₁₋₆ alkyl, C₁₋₆ alkoxy, or halogenated C₁₋₆ alkoxy;
R^{12b} represents a 5-to 20-membered monocyclic, bicyclic, or polycyclic heteroaryl group containing 1 to 4 heteroatoms selected from N, O, and S, and the heteroaryl is optionally substituted with one or more (e.g., 1-10, 1-8, 2-6, 1-5, 1-4, 1-3, or 2-5) substituents selected from the group consisting of deuterium, halogen, hydroxy, cyano, amino, C₁₋₆ alkyl, halogenated C₁₋₆ alkyl, deuterated C₁₋₆ alkyl, C₁₋₆ alkoxy, halogenated C₁₋₆ alkoxy, and any combination thereof;
R^{13g} in Formula (PBM-13) represents N or CR^{13h}, wherein R^{13h} represents hydrogen or halogen; R¹³ⁱ represents N or CR^{13j}, wherein R^{13j} represents hydrogen or halogen;
R^{13f} represents a bond, —C(O)NH—, —NHC(O)—, —S(O)₂NH—, or —NHS(O)₂—;
(R^{13a})ₚ₁₃ₐ indicates that the 6-membered ring containing R^{13g} and R¹³ⁱ in Formula (PBM-13) is optionally substituted with p13a R^{13a}, with each R^{13a} being the same or different and independently representing deuterium, halogen, hydroxy, cyano, amino, C₁₋₆ alkyl, C₁₋₆ alkoxy, halogenated C₁₋₆ alkoxy, deuterated C₁₋₆ alkyl, or halogenated C₁₋₆ alkyl;
p13a represents an integer of 0, 1 or 2;
R^{13b} represents hydrogen, halogen, C₁₋₆ alkyl, deuterated C₁₋₆ alkyl, halogenated C₁₋₆ alkyl, or—C₁₋₃ alkylene-C₃₋₆ cycloalkyl;
R^{13c} represents hydrogen, C₁₋₆ alkyl, deuterated C₁₋₆ alkyl, or halogenated C₁₋₆ alkyl, or
R^{13b} and R^{13c} together with their respective attached nitrogen and carbon atoms form an optionally substituted 5-to 7-membered nitrogen-containing heterocycle;
R^{13d} represents hydrogen or R_{c}, R^{13e} represents hydrogen or R_{c}, wherein R^{13d} and R^{13e} are not simultaneously hydrogen, and only one of R^{13d} and R^{13e} represents R_{c}, and the other represents hydrogen;
R^{14a} in Formula (PBM-14) represents a bond, C₁₋₃ alkylene, or halogenated C₁₋₃ alkylene;
R^{14b} represents optionally substituted halogen, C₁₋₆ alkyl, C₁₋₆ alkoxy, halogenated C₁₋₆ alkoxy, deuterated C₁₋₆ alkyl, halogenated C₁₋₆ alkyl, optionally substituted C₃₋₆ cycloalkyl, or optionally substituted C₁₋₆ alkyl (e.g., C₁₋₆ alkyl optionally substituted with optionally substituted aryl);
(R^{14c})ₚ₁₄ₐ indicates that the benzene ring in Formula (PBM-14) is optionally substituted with p14a R^{14c} with each R^{14c} being the same or different and independently representing deuterium, halogen, hydroxy, cyano, amino, C₁₋₆ alkyl, C₁₋₆ alkoxy, halogenated C₁₋₆ alkoxy, deuterated C₁₋₆ alkyl, or halogenated C₁₋₆ alkyl;
p14a represents an integer of 0, 1, 2, 3, or 4;
R^{15a} in Formula (PBM-15) represents C₁₋₃ alkylene or halogenated C₁₋₃ alkylene;
R^{15b} represents deuterium, C₁₋₆ alkyl, deuterated C₁₋₆ alkyl, halogenated C₁₋₆ alkyl, or optionally substituted C₃₋₆ cycloalkyl;
R^{15c} and R^{15d} each independently represent S or O;
R^{16a} in Formula (PBM-16) represents —S— or a fragment

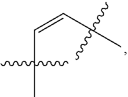 [SMILES: */C=C\*]

wherein when R^{16a} represents —S—, heteroaryl containing R^{16a} and nitrogen atom in Formula (PBM-16) is thiazolyl, and when R^{16a} represents the fragment

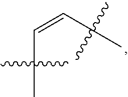 [SMILES: */C=C\*]

the heteroaryl containing R^{16a} and nitrogen atom in Formula (PBM-16) is pyridyl;
R^{16b} represents N or CH;
R^{16c} represents hydrogen or optionally substituted C₁₋₁₀ alkyl;
R^{16d} represents hydrogen, —C₁₋₃ alkylene-optionally substituted 4-to 10-membered heterocyclyl, or optionally substituted 4-to 10-membered heterocyclyl;
R^{16e} represents hydroxy, halogen, C₁₋₆ alkyl, C₁₋₆ alkoxy, halogenated C₁₋₆ alkoxy, deuterated C₁₋₆ alkyl, or halogenated C₁₋₆ alkyl;
R^{17a} in Formula (PBM-17) represents C(O)NH, NHC(O), (CH₂)₁₋₂C(O)NH, (CH₂)₁₋₂NHC(O), S(O)₂NH, or NHS(O)₂;
R^{17b} represents hydrogen, hydroxy, halogen, cyano, C₁₋₆ alkoxy, halogenated C₁₋₆ alkoxy, C₁₋₆ alkyl, deuterated C₁₋₆ alkyl, or halogenated C₁₋₆ alkyl;
R^{17c} represents hydrogen, C₁₋₆ alkyl, deuterated C₁₋₆ alkyl, or halogenated C₁₋₆ alkyl, or
R^{17b} and R^{17c}, together with their respective attached carbon and nitrogen atoms and the carbon atom on the benzene ring attached to the nitrogen atom, form an optionally substituted 4-to 6-membered heteroaromatic ring or an optionally substituted 4-to 6-membered heterocycle ring;
R^{17d} represents optionally substituted C₃₋₆ cycloalkyl, C₁₋₆ alkyl, deuterated C₁₋₆ alkyl, halogenated C₁₋₆ alkyl, or optionally substituted 4-to 20-membered heterocyclyl;
(R^{17e})ₚ₁₇ₐ indicates that pyridin-2 (1H)-one ring to which it is attached is substituted with p17a R^{17e}, with each R^{17e} being the same or different and independently representing hydroxy, halogen, cyano, C₁₋₆ alkyl, C₁₋₆ alkoxy, halogenated C₁₋₆ alkoxy, deuterated C₁₋₆ alkyl, or halogenated C₁₋₆ alkyl;
p17a represents an integer of 0, 1, 2, or 3;
ring F in Formula (PBM-17) represents arylene or 5-to 20-membered monocyclic or bicyclic heteroarylene containing from 1 to 4 heteroatoms selected from the group consisting of oxygen, nitrogen, and sulfur; and (R^{17f})_{p17b} indicates that the ring F is optionally substituted with p17b R^{17f}, with each R^{17f} being the same or different and each independently representing deuterium, halogen, hydroxy, cyano, amino, C₁₋₆ alkyl, halogenated C₁₋₆ alkyl, deuterated C₁₋₆ alkyl, C₁₋₆ alkoxy, or halogenated C₁₋₆ alkoxy;
p17b represents an integer of 0, 1, 2, 3, or 4;
R^{18f}, R^{18g}, and R^{18h} in Formula (PBM-18) each independently represent CH or N;
R^{18a} represents a bond, S, or NH;
R^{18b} represents R_{c}, optionally substituted cycloalkyl, or optionally substituted 4-to 15-membered nitrogen-containing heterocyclyl;
R^{18c} represents R_{c}, halogen, amino, hydroxy, cyano, C₁₋₆ alkyl, C₁₋₆ alkoxy, halogenated C₁₋₆ alkoxy, deuterated C₁₋₆ alkyl, halogenated C₁₋₆ alkyl, optionally substituted cycloalkyl, or optionally substituted 4-to 15-membered nitrogen-containing heterocyclyl;
wherein R^{18b} and R^{18c} do not simultaneously represent R_{c}, and only one of R^{18b} and R^{18c} represents R_{c} (i.e., one of R^{18b} and R^{18c} represents R_{c}, and the other does not represents R_{c});
(R^{18d})ₚ₁₈ₐ indicates that the 6-membered ring to which it is attached is optionally substituted with p18a R^{18d}, with each R^{18d} being the same or different and each independently representing hydroxy, amino, cyano, halogen, C₁₋₆ alkyl, C₁₋₆ alkoxy, halogenated C₁₋₆ alkoxy, deuterated C₁₋₆ alkyl, or halogenated C₁₋₆ alkyl;
p18a represents an integer of 0, 1, 2, 3, or 4;
(R^{18e})_{p18b} indicates that the 6-membered nitrogen-containing heteroaryl ring to which it is attached is optionally substituted with p18b R^{18e}, with each R^{18e} being the same or different and each independently representing hydroxy, amino, cyano, halogen, C₁₋₆ alkyl, C₁₋₆ alkoxy, halogenated C₁₋₆ alkoxy, deuterated C₁₋₆ alkyl, or halogenated C₁₋₆ alkyl;
p18b represents an integer of 0, 1 or 2;
R^{19a}, R^{19b}, and R^{19c} in Formula (PBM-19) each independently represent CH or N;
R^{19d} represents —C(O)NHR^{19h}, —NHC(O)R^{19h}, —S(O)₂NHR^{19h}, —NHS(O)₂R^{19h}, —S(O)₂R^{19h}, or —P(O)(R^{19h})₂, wherein each R^{19h} is the same or different and independently represents C₁₋₆ alkyl, deuterated C₁₋₆ alkyl, or halogenated C₁₋₆ alkyl;
(R^{19e})ₚ₁₉ₐ represents p19a R^{19e} groups, wherein each R^{19e} is the same or different and each independently represents halogen, hydroxy, cyano, amino, nitro, C₁₋₆ alkyl, halogenated C₁₋₆ alkyl, deuterated C₁₋₆ alkyl, C₁₋₆ alkoxy, or halogenated C₁₋₆ alkoxy;
p19a represents an integer of 0, 1, 2, 3, or 4;
(R^{19f}))_{p19b} represents p19b R^{19f} groups, wherein each R^{19f} is the same or different and each independently represents halogen, hydroxy, cyano, amino, nitro, C₁₋₆ alkyl, halogenated C₁₋₆ alkyl, deuterated C₁₋₆ alkyl, C₁₋₆ alkoxy, or halogenated C₁₋₆ alkoxy;
p19b represents an integer of 0, 1 or 2;
(R^{19g})_{p19c} indicates that the benzene ring to which it is attached is optionally substituted with p19c R^{19g}, with each R^{19g} independently representing halogen, hydroxy, cyano, amino, nitro, C₁₋₆ alkyl, halogenated C₁₋₆ alkyl, deuterated C₁₋₆ alkyl, C₁₋₆ alkoxy, or halogenated C₁₋₆ alkoxy;
p19c represents an integer of 0, 1, 2, 3, or 4;
Rₓ₁ in Formula (PBM-20) represents a bond or C(O), or Rₓ₁ represents —C(O)NH—Rₓ₂—C(O)—***, wherein symbol *** indicates the point of attachment to R_{c}, and Rₓ₂ represents optionally substituted C₃₋₁₅ cycloalkyl;
(R^{20a})ₚ₂₀ₐ indicates that the 1H-pyrrolo[2,3-b]pyridine ring to which it is attached is optionally substituted with p20a R^{20a}, wherein each R^{20a} is independently cyano, halogen, hydroxy, amino, nitro, C₁₋₆ alkyl, halogenated C₁₋₆ alkyl, deuterated C₁₋₆ alkyl, C₁₋₆ alkoxy, or halogenated C₁₋₆ alkoxy, and p20a represents an integer of 0, 1, 2, 3, 4, or 5;
(R^{20b})_{p20b} indicates that the pyridine ring to which it is attached is optionally substituted with p20b R^{20b} wherein each R^{20b} is independently cyano, halogen, hydroxy, nitro, C₁₋₆ alkyl, halogenated C₁₋₆ alkyl, deuterated C₁₋₆ alkyl, C₁₋₆ alkoxy, halogenated C₁₋₆ alkoxy, or NR^{20c}R^{20d}, where R^{20c} and R^{20d} are the same or different and independently represent hydrogen, C₁₋₆ alkyl, deuterated C₁₋₆ alkyl, halogenated C₁₋₆ alkyl, optionally substituted C₃₋₁₅ cycloalkyl, or optionally substituted 4-to 15-membered heterocyclyl, and p20b represents an integer of 0, 1, 2, or 3;
R^{21a} in Formula (PBM-21) represents NHC(O), C(O)NH, NHS(O)₂, or S(O)₂NH;
(R^{21b})ₚ₂₁ₐ indicates that the benzene ring to which it is attached is optionally substituted with p21a R^{21b}, wherein each R^{21b} independently represents halogen, hydroxy, cyano, amino, nitro, C₁₋₆ alkyl, halogenated C₁₋₆ alkyl, deuterated C₁₋₆ alkyl, C₁₋₆ alkoxy, or halogenated C₁₋₆ alkoxy, and p21a represents an integer of 0, 1, 2, 3, 4, or 5;
(R^{21c})_{p21b} indicates that the 1H-pyrazole ring to which it is attached is optionally substituted with p21b R^{21c}, wherein each R^{21c} is independently halogen, hydroxy, cyano, amino, nitro, C₁₋₆ alkyl, halogenated C₁₋₆ alkyl, deuterated C₁₋₆ alkyl, C₁₋₆ alkoxy, or halogenated C₁₋₆ alkoxy, and p21b represents an integer of 0, 1 or 2;
R^{22a} in Formula (PBM-22) represents N or CH, and R^{22b} represents O, S, NH, CH₂, CH(F), or C(F)₂;
R^{22c} represents a bond or optionally substituted methylene (e.g., halogenated methylene);
ring G represents C₆₋₁₅ aryl, 4-to 15-membered heterocyclyl, or 5-to 15-membered heteroaryl, and (R^{22d})ₚ₂₂ₐ indicates that the ring G to which it is attached is optionally substituted with p22a R^{22d}, wherein each R^{22d} is the same or different and each independently represents deuterium, halogen, hydroxy, cyano, amino, nitro, oxo, C₁₋₆ alkyl, halogenated C₁₋₆ alkyl, deuterated C₁₋₆ alkyl, C₁₋₆ alkoxy, or halogenated C₁₋₆ alkoxy, and p22a represents an integer of 0, 1, 2, 3, or 4;
(R^{22e})_{p22b} indicates that the 6-membered nitrogen-containing heteroaromatic ring containing R^{22a} to which it is attached is optionally substituted with p22b R^{22e}, wherein each R^{22e} independently represents deuterium, halogen, hydroxy, cyano, amino, nitro, C₁₋₆ alkoxy, halogenated C₁₋₆ alkoxy, C₁₋₆ alkyl, deuterated C₁₋₆ alkyl, or halogenated C₁₋₆ alkyl, and p22b represents an integer of 0, 1 or 2;
(R^{22f})_{p22c} indicates that the benzene ring to which it is attached is optionally substituted with p22c R^{22f}, wherein each R^{22f} is the same or different and independently represents deuterium, halogen, hydroxy, cyano, amino, nitro, C₁₋₆ alkoxy, halogenated C₁₋₆ alkoxy, C₁₋₆ alkyl, deuterated C₁₋₆ alkyl, or halogenated C₁₋₆ alkyl, and p22c represents an integer of 0, 1, 2, 3, or 4;
R^{23a} in Formula (PBM-23) represents N or CH;
R^{23b} represents NHC(O), C(O)NH, NHS(O)₂, or S(O)₂NH;
(R^{23c})ₚ₂₃ₐ indicates that the 6-membered ring containing R^{23a} is substituted with p23a R^{23c}, wherein each R^{23c} is the same or different and independently represents halogen, hydroxy, mercapto, cyano, amino, nitro, C₁₋₆ alkyl, halogenated C₁₋₆ alkyl, deuterated C₁₋₆ alkyl, C₁₋₆ alkoxy, or halogenated C₁₋₆ alkoxy, and p23a represents an integer of 0, 1, 2, 3, or 4;
ring H represents 5-to 15-membered heteroarylene, and (R^{23d})_{p23b} indicates that the ring H to which it is attached is optionally substituted with p23b R^{23d}, wherein each R^{23d} is the same or different and independently represents halogen, hydroxy, mercapto, cyano, amino, nitro, C₁₋₆ alkyl, halogenated C₁₋₆ alkyl, deuterated C₁₋₆ alkyl, hydroxy-substituted C₁₋₆ alkyl, amino-substituted C₁₋₆ alkyl, C₁₋₆ alkoxy, halogenated C₁₋₆ alkoxy, or optionally substituted C₃₋₁₅ cycloalkyl, and p23b represents an integer of 0, 1, 2, or 3;
the dashed line in Formula (PBM-24) indicates the optional presence of a double bond (i.e., the [symbol] in Formula (PBM-24) represents a single bond or a double bond);
(R^{24a})ₚ₂₄ₐ indicates that the 6-membered cycloalkyl ring to which it is attached is optionally substituted with p24a R^{24a}, wherein each R^{24a} is the same or different and independently represents halogen, hydroxy, mercapto, cyano, amino, nitro, oxo, C₁₋₆ alkyl, halogenated C₁₋₆ alkyl, deuterated C₁₋₆ alkyl, C₁₋₆ alkoxy, or halogenated C₁₋₆ alkoxy, and p24a represents an integer of 0, 1, 2, 3, 4, 5, 6, 7, or 8;
(R^{24b})_{p24b} indicates that the benzene ring to which it is attached is optionally substituted with p24b R^{24b} wherein each R^{24b} is the same or different and independently represents deuterium, halogen, hydroxy, mercapto, cyano, amino, nitro, C₁₋₆ alkyl, halogenated C₁₋₆ alkyl, deuterated C₁₋₆ alkyl, C₁₋₆ alkoxy, or halogenated C₁₋₆ alkoxy, and p24b represents an integer of 0, 1, 2, 3, 4, or 5;
(R^{24c})_{p24c} indicates that the 6-membered cycloalkyl ring to which it is attached is optionally substituted with p24c R^{24c}, wherein each R^{24c} is the same or different and independently represents halogen, cyano, oxo, C₁₋₆ alkyl, halogenated C₁₋₆ alkyl, deuterated C₁₋₆ alkyl, C₁₋₆ alkoxy, or halogenated C₁₋₆ alkoxy, and p24c represents an integer of 0, 1, 2, 3, 4, 5, 6, 7, or 8;
(R^{24d})ₚ₂₄ₐ indicates that the benzene ring to which it is attached is optionally substituted with p24d R^{24d} wherein each R^{24d} is the same or different and independently represents halogen, hydroxy, mercapto, C₁₋₆ alkyl, halogenated C₁₋₆ alkyl, deuterated C₁₋₆ alkyl, C₁₋₆ alkoxy, halogenated C₁₋₆ alkoxy, or

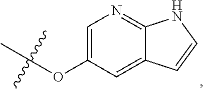 [SMILES: *Oc1cnc2[nH]ccc2c1]

and p24d represents an integer of 0, 1 or 2;
(R^{24e})ₚ₂₄ₑ indicates that the benzene ring to which it is attached is optionally substituted with p24e R^{24e} wherein each R^{24e} is the same or different and independently represents deuterium, halogen, hydroxy, mercapto, cyano, amino, nitro, —SO₂CF₃, C₁₋₆ alkyl, halogenated C₁₋₆ alkyl, deuterated C₁₋₆ alkyl, C₁₋₆ alkoxy, or halogenated C₁₋₆ alkoxy, and p24e represents an integer of 0, 1, 2, 3, or 4;
R^{24f} represents hydrogen, deuterium, halogen, hydroxy, mercapto, cyano, amino, nitro, C₁₋₆ alkyl, halogenated C₁₋₆ alkyl, deuterated C₁₋₆ alkyl, C₁₋₆ alkoxy, halogenated C₁₋₆ alkoxy, or

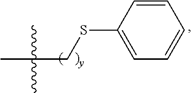

wherein y represents an integer of 1, 2, or 3;
(R^{25a})ₚ₂₅ₐ in Formula (PBM-25) represents p25a R^{25a} groups, wherein each R^{25a} is the same or different and independently represents halogen, hydroxy, mercapto, cyano, amino, nitro, C₁₋₆ alkyl, halogenated C₁₋₆ alkyl, deuterated C₁₋₆ alkyl, C₁₋₆ alkoxy, or halogenated C₁₋₆ alkoxy, and p25a represents an integer of 0, 1, 2, 3, 4, or 5;
(R^{25b})_{p25b} indicates that the benzene ring to which it is attached is optionally substituted with p25b R^{25b}, wherein each R^{25b} is the same or different and independently represents deuterium, halogen, hydroxy, mercapto, cyano, amino, nitro, C₁₋₆ alkyl, halogenated C₁₋₆ alkyl, deuterated C₁₋₆ alkyl, C₁₋₆ alkoxy, or halogenated C₁₋₆ alkoxy, and p25b represents an integer of 0, 1, 2, 3, or 4, and
R^{25c} represents C₁₋₆ alkylene or halogenated C₁₋₆ alkylene;
(R^{26a})ₚ₂₆ₐ in Formula (PBM-26) indicates that the benzene ring to which it is attached is optionally substituted with p26a R^{26a}, wherein each R^{26a} is the same or different and independently represents deuterium, halogen, hydroxy, amino, cyano, C₁₋₆ alkoxy, halogenated C₁₋₆ alkoxy, C₁₋₆ alkyl, deuterated C₁₋₆ alkyl, or halogenated C₁₋₆ alkyl;
p26a represents an integer of 0, 1, 2, 3, or 4;
(R^{26b})_{p26b} indicates that the 4-oxo-3,4-dihydroquinazoline ring to which it is attached is substituted with p26b R^{26b}, wherein each R^{26b} is the same or different and independently represents halogen, hydroxy, amino, cyano, C₁₋₆ alkoxy, halogenated C₁₋₆ alkoxy, C₁₋₆ alkyl, deuterated C₁₋₆ alkyl, or halogenated C₁₋₆ alkyl; p26b represents an integer of 1, 2, 3, or 4;
(R^{27a})ₚ₂₇ₐ in Formula (PBM-27) indicates that the 6-membered ring containing R^{27d} to which it is attached is optionally substituted with p27a R^{27a}, wherein each R^{27a} is the same or different and independently represents deuterium, halogen, hydroxy, amino, cyano, C₁₋₆ alkoxy, halogenated C₁₋₆ alkoxy, C₁₋₆ alkyl, deuterated C₁₋₆ alkyl, or halogenated C₁₋₆ alkyl;
p27a represents an integer of 0, 1, 2, or 3;
(R^{27b})_{p27b} represents p27b R^{27b} groups, wherein each R^{27b} is the same or different and independently represents deuterium, halogen, hydroxy, amino, cyano, C₁₋₆ alkoxy, halogenated C₁₋₆ alkoxy, C₁₋₆ alkyl, deuterated C₁₋₆ alkyl, halogenated C₁₋₆ alkyl, or optionally substituted 5-to 15-membered aryl;
p27b represents an integer of 0, 1, 2, or 3;
R^{27c} and R^{27d} each independently represents N or CH;
R^{27e} represents hydrogen or a group having the following structure of Formula (III):

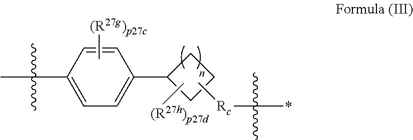

wherein (R^{27g})_{p27c} indicates that the benzene ring to which it is attached is optionally substituted with p27c R^{27g}, with each R^{27g} being the same or different and independently representing hydrogen, deuterium, halogen, hydroxy, amino, cyano, C₁₋₆ alkoxy, halogenated C₁₋₆ alkoxy, C₁₋₆ alkyl, deuterated C₁₋₆ alkyl, or halogenated C₁₋₆ alkyl;
p27c represents an integer of 0, 1, 2, 3, or 4;
n represents an integer of 1, 2, 3, 4, or 5;
(R^{27h})ₚ₂₇ₐ represents p27d R^{27h} groups, wherein each R^{27h} is the same or different and independently represents hydrogen, deuterium, halogen, hydroxy, amino, cyano, C₁₋₆ alkoxy, halogenated C₁₋₆ alkoxy, C₁₋₆ alkyl, deuterated C₁₋₆ alkyl, or halogenated C₁₋₆ alkyl;
p27d represents an integer of 0, 1, 2, 3, 4, 5, 6, or 7, and
symbol * indicates the point of attachment to LIN;
R^{27f} represents hydrogen or R_{c};
wherein when R^{27e} represents hydrogen, R^{27f} represents R_{c}; when R^{27f} represents hydrogen, R^{27e} represents the group having the structure of Formula (III);
R^{28a} represents O, S, or NH;
(R^{28b})ₚ₂₈ₐ indicates that the nitrogen-containing 5-membered ring to which it is attached is optionally substituted with p28a R^{28b}, wherein each R^{28b} is the same or different and independently represents deuterium, halogen, hydroxy, amino, cyano, C₁₋₆ alkoxy, halogenated C₁₋₆ alkoxy, C₁₋₆ alkyl, deuterated C₁₋₆ alkyl, or halogenated C₁₋₆ alkyl;
p28a represents an integer of 0, 1 or 2;
(R^{28c})_{p28b} indicates that the benzene ring to which it is attached is optionally substituted with p28b R^{28c} wherein each R^{28c} is the same or different and independently represents deuterium, halogen, hydroxy, amino, cyano, C₁₋₆ alkoxy, halogenated C₁₋₆ alkoxy, C₁₋₆ alkyl, deuterated C₁₋₆ alkyl, or halogenated C₁₋₆ alkyl;
p28b represents an integer of 0, 1, 2, 3, or 4;
(R^{28d})_{p28c} indicates that the benzene ring to which it is attached is optionally substituted with p28c R^{28d} A wherein each R^{28d} is the same or different and independently represents deuterium, halogen, hydroxy, amino, cyano, C₁₋₆ alkoxy, halogenated C₁₋₆ alkoxy, C₁₋₆ alkyl, deuterated C₁₋₆ alkyl, halogenated C₁₋₆ alkyl, or optionally substituted C₃₋₁₅ cycloalkyl;
p28c represents an integer of 0, 1, 2, 3, 4, or 5;
R^{29a} represents S(O)₂ or a bond;
R^{29b} represents NH, NHC(O), or C(O)NH;
R^{29c} represents CH or N;
R^{29d} represents N(R^{29h}), wherein R^{29h} represents H, —C(O)— optionally substituted aryl, or —C(O)— optionally substituted heteroaryl, or
R^{29d} represents fragment

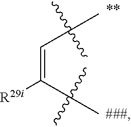

wherein symbol ** indicates the point of attachment to the adjacent nitrogen atom, symbol ###indicates the point of attachment to the adjacent carbon atom, and R²⁹ⁱ represents deuterium, hydrogen, halogen, C₁₋₆ alkyl, halogenated C₁₋₆ alkyl, or deuterated C₁₋₆ alkyl;
(R^{29e})ₚ₂₉ₐ indicates that the benzene ring to which it is attached is optionally substituted with p29a R^{29e}, wherein each R^{29e} is the same or different and independently represents deuterium, halogen, hydroxy, amino, cyano, C₁₋₆ alkoxy, halogenated C₁₋₆ alkoxy, C₁₋₆ alkyl, deuterated C₁₋₆ alkyl, or halogenated C₁₋₆ alkyl; p29a represents an integer of 0, 1, 2, 3, or 4;
(R^{29f})_{p29b} indicates that the nitrogen-containing ring to which it is attached is substituted by p29b R^{29f} substituents, wherein each R^{29f} is the same or different and independently represents deuterium, amino, halogen, hydroxy, cyano, C₁₋₆ alkoxy, halogenated C₁₋₆ alkoxy, C₁₋₆ alkyl, deuterated C₁₋₆ alkyl, halogenated C₁₋₆ alkyl, —NH-optionally substituted aryl, or—NH-optionally substituted heteroaryl;
p29b represents an integer of 1 or 2;
(R^{30a})ₚ₃₀ₐ in Formula (PBM-30) indicates that that the fused tricyclic ring to which it is attached is optionally substituted by p30a R^{30a} substituents, wherein each R^{30a} is the same or different and independently represents deuterium, halogen, hydroxy, amino, cyano, C₁₋₆ alkoxy, halogenated C₁₋₆ alkoxy, C₁₋₆ alkyl, deuterated C₁₋₆ alkyl, or halogenated C₁₋₆ alkyl;
p30a represents an integer of 0, 1, 2, 3, 4, 5, or 6;
[symbol] in Formula (PBM-30) represents a single bond or a double bond; fragment

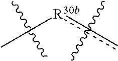

of the fused tricyclic ring in Formula (PBM-30) represents

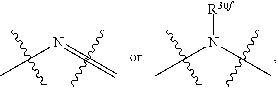

wherein R^{30f} represents hydrogen, C₁₋₆ alkyl, deuterated C₁₋₆ alkyl, or halogenated C₁₋₆ alkyl;
wherein when the fragment

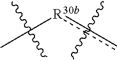

represents

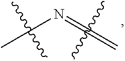 [SMILES: *N=*]

fragment

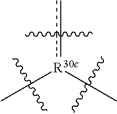

represents

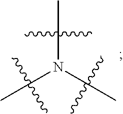 [SMILES: *N(*)*]

when the fragment

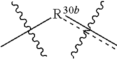

represents

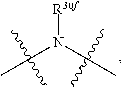

fragment

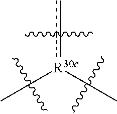

represents

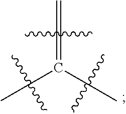 [SMILES: *C(*)=*]

R^{30d} and R^{30e} are the same or different and independently represent N or CH;
R^{31a} in Formula (PBM-31) represents O, S, or NH;
(R^{31b})ₚ₃₁ₐ indicates that the 5-membered ring containing R^{31a} to which it is attached is optionally substituted with p31a R^{31b}, wherein each R^{31b} is the same or different and independently represents deuterium, halogen, hydroxy, amino, cyano, C₁₋₆ alkoxy, halogenated C₁₋₆ alkoxy, C₁₋₆ alkyl, deuterated C₁₋₆ alkyl, or halogenated C₁₋₆ alkyl;
p31a represents an integer of 0, 1, 2, or 3;
(R^{31c})_{p31b} indicates that the diazaheterocyclyl to which it is attached is optionally substituted by p31b R^{31c} substituents, wherein each R^{31c} is the same or different and independently represents deuterium, halogen, hydroxy, amino, cyano, C₁₋₆ alkoxy, halogenated C₁₋₆ alkoxy, C₁₋₆ alkyl, deuterated C₁₋₆ alkyl, or halogenated C₁₋₆ alkyl;
p31b represents an integer of 0, 1, 2, 3, 4, 5, 6, 7, 8, 9, or 10;
Z1 represents an integer of 1, 2, or 3;
Z2 represents an integer of 1, 2, 3, 4, 5, or 6;
R^{32a} in Formula (PBM-32) represents O, S, or NH;
R^{32b} represents hydrogen, C₁₋₆ alkyl, deuterated C₁₋₆ alkyl, or halogenated C₁₋₆ alkyl;
Z3 represents an integer of 1, 2, or 3;
(R^{32c})ₚ₃₂ₐ indicates that the benzene ring to which it is attached is optionally substituted with p32a R^{32c} substituents, wherein each R^{32c} is the same or different and independently represents deuterium, halogen, hydroxy, amino, cyano, C₁₋₆ alkoxy, halogenated C₁₋₆ alkoxy, C₁₋₆ alkyl, deuterated C₁₋₆ alkyl, or halogenated C₁₋₆ alkyl;
p32a represents an integer of 0, 1, 2, 3, 4, or 5;
(R^{32d})_{p32b} indicates that the 5-membered ring containing R^{32a} to which it is attached is optionally substituted with p32b R^{32d} substituents, wherein each R^{32d} is the same or different and independently represents deuterium, halogen, hydroxy, amino, cyano, C₁₋₆ alkoxy, halogenated C₁₋₆ alkoxy, C₁₋₆ alkyl, deuterated C₁₋₆ alkyl, or halogenated C₁₋₆ alkyl;
p32b represents an integer of 0, 1 or 2;
(R^{32e})_{p32c} indicates that the pyrazole ring to which it is attached is optionally substituted by p32c R^{32e} substituents, wherein each R^{32e} is the same or different and independently represents deuterium, halogen, hydroxy, amino, cyano, C₁₋₆ alkoxy, halogenated C₁₋₆ alkoxy, C₁₋₆ alkyl, deuterated C₁₋₆ alkyl, or halogenated C₁₋₆ alkyl;
p32c represents an integer of 0, 1 or 2;
(R^{33a})ₚ₃₃ₐ indicates that the benzene ring to which it is attached is optionally substituted with p33a R^{33a} substituents, wherein each R^{33a} is the same or different and independently represents deuterium, halogen, hydroxy, amino, cyano, C₁₋₆ alkoxy, halogenated C₁₋₆ alkoxy, C₁₋₆ alkyl, deuterated C₁₋₆ alkyl, or halogenated C₁₋₆ alkyl;
p33a represents an integer of 0, 1, 2, 3, 4, or 5;
R^{33b}, R^{33c}, R^{33d}, R^{33e}, R^{33f}, R^{33g}, and R^{33h} in Formula (PBM-33) are the same or different and independently represent hydrogen, deuterium, halogen, hydroxy, amino, cyano, C₁₋₆ alkoxy, halogenated C₁₋₆ alkoxy, C₁₋₆ alkyl, deuterated C₁₋₆ alkyl, or halogenated C₁₋₆ alkyl; and
R_{c} in each general formula independently represents a bond, —O—, —N(Rₐ)—, —NHC(O)—*, —C(O)NH—*, —N(R_{d})—R_{f}—N(Rₑ)—*, —R_{f}—C(O)NH—*, —R_{f}—NHC(O)—*, —R_{f}—C(O)O—*, —R_{f}—OC(O)—*, —C(O)O—*, —OC(O)—*, —R_{f}—, —R_{f}—N(R_{d})—*—O—R_{f}—N(R_{d})—*,

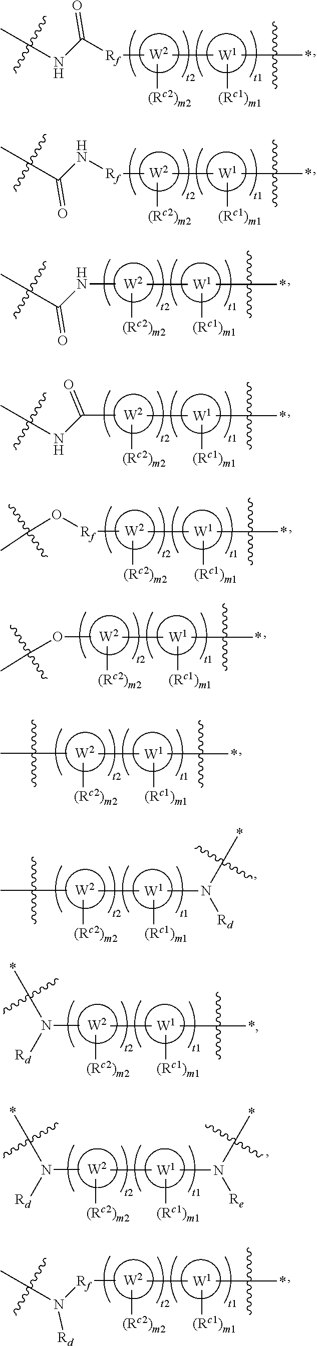

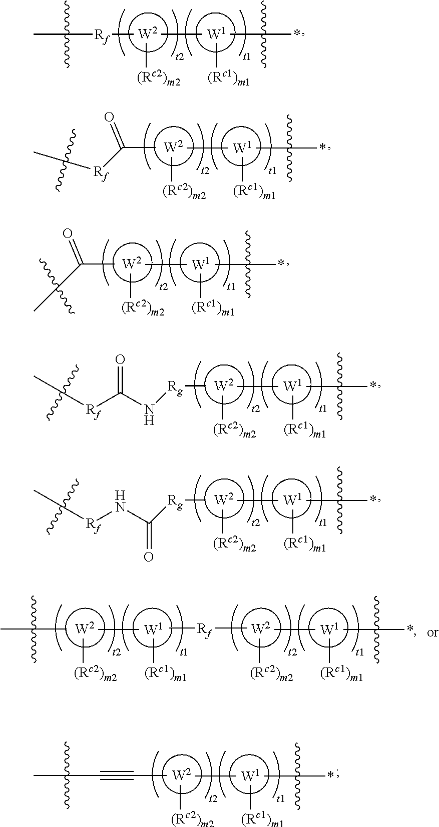

wherein each ring W¹ is the same or different and each independently represents nitrogen-containing heterocyclylene, t1 represents an integer of 1 or 2, and (R^{c1})ₘ₁ indicates that each ring W¹ is independently optionally substituted with m1 Rel groups, wherein each Rel is independently deuterium, C₁₋₆ alkyl, deuterated C₁₋₆ alkyl, halogenated C₁₋₆ alkyl, C₁₋₆ alkoxy, halogen, amino, hydroxy, mercapto, cyano, carboxyl, C₂₋₆ alkynyl, C₂₋₆ alkenyl, optionally deuterated C₃₋₆ cycloalkyl, or R^{c4}—R_{c3}—, wherein R_{c3} is optionally substituted C₁₋₆ alkylene or optionally substituted C₂₋₆ alkenylene, and R^{c4} represents halogen, R_{c5}R^{c6}N—, hydroxy, carboxyl, ethynyl, or vinyl, wherein R_{c5} and R_{c6} are the same or different and independently represent hydrogen or C₁₋₃ alkyl, and m1 represents an integer of 0-20;
each ring W² is the same or different and each independently represents heterocyclylene, cycloalkylene, arylene, or heteroarylene, t2 represents an integer of 0 or 1, and (R_{c2})ₘ₂ indicates that each ring W² is independently optionally substituted with m2 R_{c2} groups, wherein each R_{c2} is independently deuterium, C₁₋₆ alkyl, deuterated C₁₋₆ alkyl, halogenated C₁₋₆ alkyl, C₁₋₆ alkoxy, halogen, amino, hydroxy, mercapto, cyano, carboxyl, C₂₋₆ alkynyl, C₂₋₆ alkenyl, optionally deuterated C₃₋₆ cycloalkyl, or R_{c8}—R_{c7}—, wherein R_{c7} is optionally substituted C₁₋₆ alkylene or optionally substituted C₂₋₆ alkenylene, and R_{c8} represents halogen, R_{c9}R^{c10}N—, hydroxy, carboxyl, ethynyl, or vinyl, wherein Rₑ₉ and Rₑ₁₀ are the same or different and independently represent hydrogen or C₁₋₃ alkyl, and m2 represents an integer of 0-20; and
R_{d} and Rₑ each independently represent hydrogen or C₁₋₆ alkyl, R_{f} and R_{g} each independently represent optionally substituted C₁₋₆ alkylene or optionally substituted C₂₋₆ alkenylene, and symbol * indicates the point of attachment to LIN.

In the embodiments of the present disclosure, the number of substituents is not theoretically limited in any way, or is automatically limited by the size of the building units (i.e., the total number of replaceable hydrogen atoms in the building units), or as clearly defined herein.

In some embodiments, PBM represents a small molecule ligand of EGFR.

In some embodiments, PBM represents a structure of Formula (PBM-1):

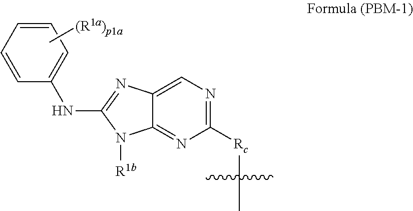

wherein (R^{1a})ₚ₁ₐ indicates that the benzene ring to which it is attached is substituted with p1a R^{1a} groups, with each R^{1a} being the same or different and independently representing deuterium, halogen (e.g., fluorine, chlorine, bromine, or iodine), hydroxy, C₁₋₆ alkyl (e.g., C₁₋₄ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, or perdeuterated C₁₋₄ alkyl, such as CD₃, CD₃CD₂—, CD₃CD₂CD₂— etc.), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), C₁₋₆ alkoxy (e.g., C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), NO₂, NH₂, or cyano;
p1a represents an integer of 0, 1, 2, 3, 4, or 5;
R^{1b} represents substituted or unsubstituted C₃₋₁₅ cycloalkyl (e.g., substituted or unsubstituted C₃₋₁₀ cycloalkyl, or C₃₋₆ cycloalkyl, such as cyclopropyl, cyclobutyl, cyclopentyl, or cyclohexyl). In some embodiments, the C₃₋₁₅ cycloalkyl is optionally substituted with one or more (e.g., 1-3, 1, 2, or 3) substituents selected from the group consisting of deuterium, hydroxy, amino, mercapto, nitro, halogen (e.g., fluorine, chlorine, bromine, or iodine), cyano, C₁₋₆ alkyl (e.g., C₁₋₄ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, or perdeuterated C₁₋₄ alkyl, such as CD₃, CD₃CD₂—, CD₃CD₂CD₂— etc.), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), optionally halogenated C₃₋₆ cycloalkyl, C₁₋₆ alkoxy (e.g., C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), C₁₋₆ alkyl-NH—, NH₂-C₁₋₆ alkylene, C₁₋₆ alkyl-NHC(O)—, C₁₋₆ alkyl-C(O)NH—, and any combination thereof.

In some embodiments, PBM represents the structure of Formula (PBM-1-1):

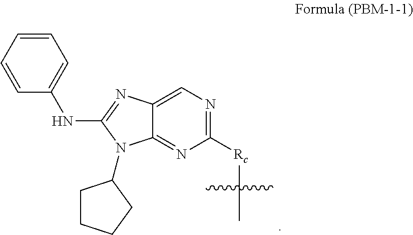

In some embodiments, PBM represents a structure of Formula (PBM-2-1A), Formula (PBM-2-1B), Formula (PBM-2-1C), or Formula (PBM-2-1D)(which are non-limiting examples of the structure of Formula (PBM-2)):

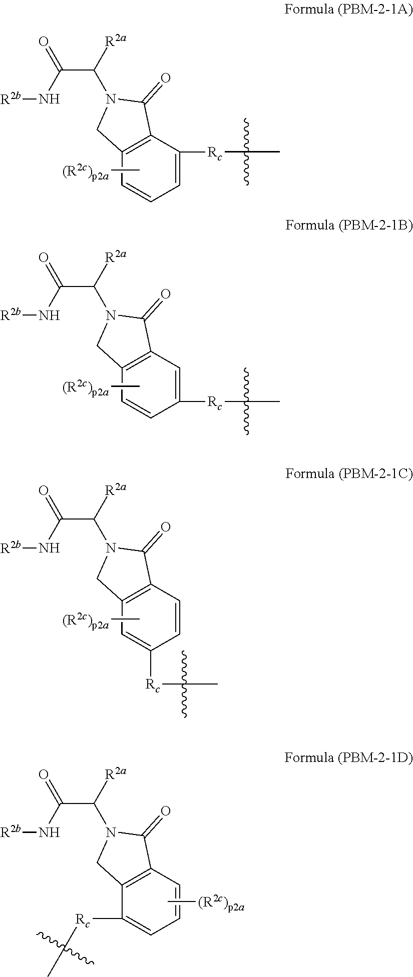

wherein in each of Formula (PBM-2-1A), Formula (PBM-2-1B), Formula (PBM-2-1C), and Formula (PBM-2-1D),
R^{2a} represents C₆₋₁₀ aryl or 5-to 20-membered heteroaryl (e.g., 5-to 20-membered heteroaryl containing one or two 5-to 7-membered rings and 1 to 4 heteroatoms selected from N, O, and S), wherein the C₆₋₁₀ aryl and 5-to 20-membered heteroaryl are each independently optionally substituted with one or more (e.g., 1-6, such as 1, 2, 3, 4, 5, or 6) substituents independently selected from the group consisting of deuterium, halogen (e.g., fluorine, chlorine, bromine, or iodine), hydroxy, cyano, amino, nitro, C₁₋₆ alkyl (e.g., C₁₋₆ alkyl, C₁₋₄ alkyl, or C₁₋₃ alkyl, such as methyl, ethyl, propyl, isopropyl, butyl, sec-butyl, tert-butyl, pentyl, or hexyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), C₁₋₆ alkoxy (e.g., C₁₋₅ alkoxy, or C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), and any combination thereof;
R^{2b} represents C₆₋₁₀ aryl or 5-to 20-membered heteroaryl (e.g., 5-to 20-membered heteroaryl containing one or two 5-to 7-membered rings and 1 to 4 heteroatoms selected from N, O, and S), wherein the C₆₋₁₀ aryl and 5-to 20-membered heteroaryl are each independently optionally substituted with one or more (e.g., 1-6, such as 1, 2, 3, 4, 5, or 6) substituents R^{2d} independently selected from the group consisting of deuterium, halogen (e.g., fluorine, chlorine, bromine, or iodine), hydroxy, cyano, amino, nitro, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), C₁₋₆ alkoxy (e.g., C₁₋₄ alkoxy, such as methoxy, ethoxy, propoxy, isopropoxy, n-butoxy, sec-butoxy, or tert-butoxy), and halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), and
(R^{2c})ₚ₂ₐ indicates that the isoindoline ring to which it is attached is optionally substituted with p2a R^{2c}, wherein each R^{2c} is the same or different and independently represents deuterium, halogen (e.g., fluorine, chlorine, bromine, or iodine), hydroxy, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), NO₂, NH₂, C₁₋₆ alkoxy (e.g., C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), or cyano, and p2a represents an integer of 0, 1, 2, or 3.

In some embodiments, R^{2a} represents C₆₋₁₀ aryl, such as but not limited to phenyl or naphthyl, each of which is optionally substituted with one or more (e.g., 1-6, such as 1, 2, 3, 4, 5, or 6) substituents independently selected from the group consisting of deuterium, halogen (e.g., fluorine, chlorine, bromine, or iodine), hydroxy, cyano, amino, nitro, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), C₁₋₆ alkoxy (e.g., C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), and any combination thereof.

In some embodiments, R^{2a} represents 5-to 20-membered heteroaryl, e.g., 5-to 20-membered heteroaryl containing one or two 5-to 7-membered rings and 1 to 4 heteroatoms selected from N, O, and S. In a sub-embodiment, R^{2a} represents 5-to 14-membered monocyclic or bicyclic aromatic ring group containing one or more (e.g., from 1 to 6, or from 1 to 5, or from 1 to 4, or from 1 to 3) heteroatoms selected from the group consisting of oxygen, nitrogen, and sulfur. In a sub-embodiment, examples of heteroaryl include, but are not limited to, furanyl, oxazolyl, isoxazolyl, oxadiazolyl, thienyl, thiazolyl, isothiazolyl, thiadiazolyl, pyrrolyl, imidazolyl, pyrazolyl, triazolyl, pyridyl, pyrimidinyl, pyridazinyl, pyrazinyl, indolyl, isoindolyl, benzofuranyl, isobenzofuranyl, benzothienyl, indazolyl, benzimidazolyl, benzoxazolyl, benzisoxazolyl, benzothiazolyl, benzisothiazolyl, benzotriazolyl, benzo[2,1,3]oxadiazolyl, benzo[2,1,3]thiadiazolyl, benzo[1,2,3]thiadiazolyl, quinolinyl, isoquinolinyl, naphthyridinyl, cinnolinyl, quinazolinyl, quinoxalinyl, phthalazinyl, pyrazolo[1,5-alpyridyl, pyrazolo[1,5-a]pyrimidinyl, imidazo[1,2-a]pyridyl, 1H-pyrrolo[3,2-b]pyridyl, 1H-pyrrolo[2,3-b]pyridyl, pyrrolo[2,1-b]thiazolyl, imidazo[2,1-b]thiazolyl, imidazo[1,2-b]pyridazinyl or 6,7-dihydro-5H-pyrrolo[1,2-c]imidazolyl. The heteroaryl group is optionally substituted with one or more (e.g., 1-6, such as 1, 2, 3, 4, 5, or 6) substituents independently selected from the group consisting of deuterium, halogen (e.g., fluorine, chlorine, bromine, or iodine), hydroxy, cyano, amino, nitro, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), C₁₋₆ alkoxy (e.g., C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), and any combination thereof.

In some embodiments, R^{2b} represents C₆₋₁₀ aryl, such as but not limited to phenyl or naphthyl, each of which is optionally substituted with one or more (e.g., 1-6, such as 1, 2, 3, 4, 5, or 6) substituents R^{2d} as defined above.

In some embodiments, R^{2b} represents heteroaryl containing one or two 5-to 7-membered rings and 1 to 4 heteroatoms selected from N, O, and S. In a sub-embodiment, R^{2b} represents 5-to 14-membered monocyclic or bicyclic aromatic ring group containing one or more (e.g., from 1 to 6, or from 1 to 5, or from 1 to 4, or from 1 to 3) heteroatoms selected from the group consisting of oxygen, nitrogen, and sulfur. In a sub-embodiment, examples of heteroaryl include, but are not limited to, furanyl, oxazolyl, isoxazolyl, oxadiazolyl, thienyl, thiazolyl, isothiazolyl, thiadiazolyl, pyrrolyl, imidazolyl, pyrazolyl, triazolyl, pyridyl, pyrimidinyl, pyridazinyl, pyrazinyl, indolyl, isoindolyl, benzofuranyl, isobenzofuranyl, benzothienyl, indazolyl, benzimidazolyl, benzoxazolyl, benzisoxazolyl, benzothiazolyl, benzisothiazolyl, benzotriazolyl, benzo[2,1,3]oxadiazolyl, benzo[2,1,3]thiadiazolyl, benzo[1,2,3]thiadiazolyl, quinolinyl, isoquinolinyl, naphthyridinyl, cinnolinyl, quinazolinyl, quinoxalinyl, phthalazinyl, pyrazolo[1,5-a]pyridyl, pyrazolo[1,5-alpyrimidinyl, imidazo[1,2-a]pyridyl, 1H-pyrrolo[3,2-b]pyridyl, 1H-pyrrolo[2,3-b]pyridyl, pyrrolo[2,1-b]thiazolyl, imidazo[2,1-b]thiazolyl, and imidazo[1,2-b]pyridazinyl. The heteroaryl group is optionally substituted with one or more (e.g., 1-6, such as 1, 2, 3, 4, 5, or 6) substituents R^{2d} as defined above.

In some embodiments, PBM represents a structure of Formula (PBM-2-1), Formula (PBM-2-2), Formula (PBM-2-3), Formula (PBM-2-4), or Formula (PBM-2-5):

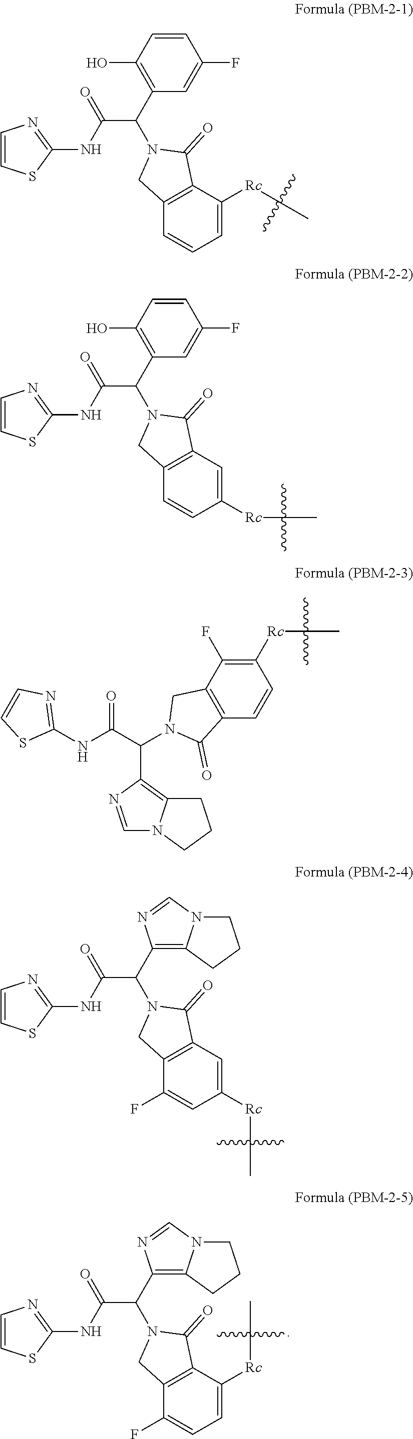

In some embodiments, PBM represents a structure of Formula (PBM-3):

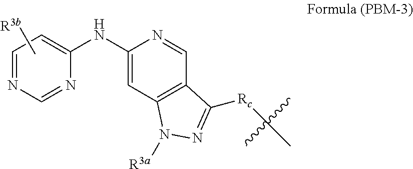

wherein R^{3a} represents hydrogen, C₁₋₁₀ alkyl (e.g., C₁₋₈ alkyl, C₁₋₆ alkyl, or C₁₋₄ alkyl, such as methyl, ethyl, propyl, isopropyl

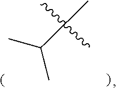 [SMILES: *C(C)C]

butyl, sec-butyl, tert-butyl, pentyl, hexyl, heptyl, or octyl), deuterated C₁₋₁₀ alkyl (e.g., perdeuterated C₁₋₈ alkyl, perdeuterated C₁₋₆ alkyl, or perdeuterated C₁₋₄ alkyl, such as CD₃, CD₃CD₂—, CD₃CD₂CD₂— etc.), or halogenated C₁₋₁₀ alkyl (e.g., halogenated C₁₋₈ alkyl, or halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), and
R^{3b} represents

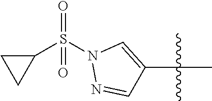 [SMILES: *c1cnn(S(=O)(=O)C2CC2)c1]

or 4-to 12-membered nitrogen-containing heterocyclyl, wherein the 4-to 12-membered nitrogen-containing heterocyclyl is optionally substituted with one or more substituents independently selected from the group consisting of deuterium, halogen (e.g., fluorine, chlorine, bromine, or iodine), hydroxy, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), halogenated C₁₋₆ alkyl (e.g., halogenated C₁₋₅ alkyl, or halogenated C₁₋₃ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), C₁₋₆ alkoxy (e.g., C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), deuterated C₁₋₆ alkoxy (e.g., perdeuterated C₁₋₄ alkoxy, such as CD₃—O—, CD₃CD₂—O—, or CD₃CD₂CD₂—O—), NO₂, NH₂, cyano, and any combination thereof. In some sub-embodiments, examples of 4-to 12-membered nitrogen-containing heterocyclyl include, but are not limited to, azetidinyl, pyrrolidinyl, imidazolidinyl, pyrazolidyl, oxazolidinyl, thiazolidinyl, piperidinyl, piperazinyl, morpholinyl, thiomorpholinyl, dioxacyclohexyl, azacycloheptyl, diazacycloheptanyl (e.g., 1,4-diazacycloheptan-1-yl), azacyclooctyl, diazacyclooctyl, azabicyclo[3.1.1]heptanyl, azabicyclo[2.2.1]heptanyl, azabicyclo[3.2.1]octanyl, azabicyclo[2.2.2]octanyl, diazabicyclo[3.1.1]heptanyl, diazabicyclo[2.2.1]heptanyl, diazabicyclo[3.2.1]octanyl (e.g., 3,8-diazabicyclo[3.2.1]octan-3-yl), and diazabicyclo[2.2.2]octanyl (e.g., 2,5-diazabicyclo[2.2.2]octan-2-yl).

In some embodiments, PBM represents a structure of Formula (PBM-3-1) or Formula (PBM-3-2):

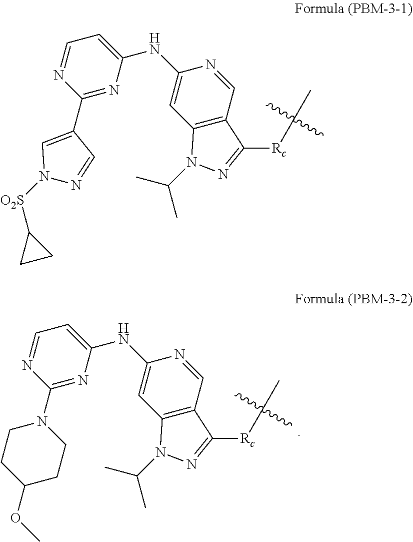

In some embodiments. PBM represents a structure of Formula (PBM-19):

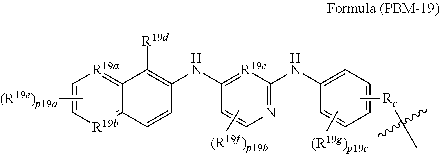

wherein R^{19a}, R^{19b}, and R^{19c} each independently represent CH or N;
R^{19d} represents —C(O)NHR^{19h}, —NHC(O)R^{19h}, —S(O)₂NHR^{19h}, —NHS(O)₂R^{19h}, —S(O)₂R^{19h}, or —P(O)(R^{19h})₂, wherein each R^{19h} is the same or different and independently represents C₁₋₆ alkyl (e.g., C₁₋₄ alkyl, such as methyl, ethyl, propyl, isopropyl, butyl, sec-butyl, tert-butyl, pentyl, or hexyl), deuterated C₁₋₆ alkyl (e.g., perdeuterated C₁₋₅ alkyl, perdeuterated C₁₋₄ alkyl, or perdeuterated C₁₋₃ alkyl, such as CD₃, CD₃CD₂—, CD₃CD₂CD₂-etc.), or halogenated C₁₋₆ alkyl (e.g., halogenated C₁₋₅ alkyl, or halogenated C₁₋₃ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—);
(R^{19e})ₚ₁₉ₐ represents p19a R^{19e} groups, wherein each R^{19e} is the same or different and independently represents halogen (e.g., fluorine, chlorine, bromine, or iodine), hydroxy, cyano, amino, nitro, C₁₋₆ alkyl (e.g., C₁₋₄ alkyl, such as methyl, ethyl, propyl, isopropyl, butyl, sec-butyl, tert-butyl, pentyl, or hexyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), deuterated C₁₋₆ alkyl (e.g., perdeuterated C₁₋₅ alkyl, perdeuterated C₁₋₄ alkyl, or perdeuterated C₁₋₃ alkyl, such as CD₃, CD₃CD₂—, CD₃CD₂CD₂— etc.), C₁₋₆ alkoxy (e.g., C₁₋₅ alkoxy, C₁₋₄ alkoxy, or C₁₋₃ alkoxy, such as methoxy, ethoxy, propoxy, isopropoxy, n-butoxy, sec-butoxy, or tert-butoxy), or halogenated C₁₋₆ alkoxy (e.g., halogenated C₁₋₄ alkoxy, such as trifluoromethoxy);
p19a represents an integer of 0, 1, 2, 3, or 4;
(R^{19f})_{p19b} represents p19b R^{19f} groups, wherein each R^{19f} is the same or different and independently represents halogen (e.g., fluorine, chlorine, bromine, or iodine), hydroxy, cyano, amino, nitro, C₁₋₆ alkyl (e.g., C₁₋₄ alkyl, such as methyl, ethyl, propyl, isopropyl, butyl, sec-butyl, tert-butyl, pentyl, or hexyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), deuterated C₁₋₆ alkyl (e.g., perdeuterated C₁₋₅ alkyl, perdeuterated C₁₋₄ alkyl, or perdeuterated C₁₋₃ alkyl, such as CD₃, CD₃CD₂—, CD₃CD₂CD₂— etc.), C₁₋₆ alkoxy (e.g., C₁₋₅ alkoxy, C₁₋₄ alkoxy, or C₁₋₃ alkoxy, such as methoxy, ethoxy, propoxy, isopropoxy, n-butoxy, sec-butoxy, or tert-butoxy), or halogenated C₁₋₆ alkoxy (e.g., halogenated C₁₋₄ alkoxy, such as trifluoromethoxy);
p19b represents an integer of 0, 1 or 2;
(R^{19g})_{p19c} indicates that the benzene ring to which it is attached is optionally substituted with p19c R^{19g}, with each R^{19g} independently representing halogen (e.g., fluorine, chlorine, bromine, or iodine), hydroxy, cyano, amino, nitro, C₁₋₆ alkyl (e.g., C₁₋₄ alkyl, such as methyl, ethyl, propyl, isopropyl, butyl, sec-butyl, tert-butyl, pentyl, or hexyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), deuterated C₁₋₆ alkyl (e.g., perdeuterated C₁₋₅ alkyl, perdeuterated C₁₋₄ alkyl, or perdeuterated C₁₋₃ alkyl, such as CD₃, CD₃CD₂—, CD₃CD₂CD₂— etc.), C₁₋₆ alkoxy (e.g., C₁₋₅ alkoxy, C₁₋₄ alkoxy, or C₁₋₃ alkoxy, such as methoxy, ethoxy, propoxy, isopropoxy, n-butoxy, sec-butoxy, or tert-butoxy), or halogenated C₁₋₆ alkoxy (e.g., halogenated C₁₋₄ alkoxy, such as trifluoromethoxy); and
p19c represents an integer of 0, 1, 2, 3, or 4.

In some embodiments, R^{19a} and R^{19b} each independently represent CH or N. In some sub-embodiments, R^{19a} represents CH, and R^{19b} represents N. In some sub-embodiments, R^{19a} represents CH, and R^{19b} represents CH. In some sub-embodiments, R^{19a} represents N, and R^{19b} represents CH. In some sub-embodiments, R^{19a} represents N, and R^{19b} represents N.

In some embodiments, R^{19c} represents CH or N. In some sub-embodiments, R^{19c} represents N. In some sub-embodiments, R^{19c} represents CH.

In some embodiments, R^{19d} represents-C(O)NHR^{19h}, —NHC(O)R^{19h}, —S(O)₂NHR^{19h}, —NHS(O)₂R^{19h}, —S(O)₂R^{19h}, or —P(O)(R^{19h})₂, wherein each R^{19h} is the same or different and independently represents C₁₋₆ alkyl (e.g., C₁₋₄ alkyl, such as methyl, ethyl, propyl, isopropyl, butyl, sec-butyl, tert-butyl, pentyl, or hexyl), deuterated C₁₋₆ alkyl (e.g., perdeuterated C₁₋₅ alkyl, perdeuterated C₁₋₄ alkyl, or perdeuterated C₁₋₃ alkyl, such as CD₃, CD₃CD₂—, CD₃CD₂CD₂— etc.), or halogenated C₁₋₆ alkyl (e.g., halogenated C₁₋₅ alkyl, or halogenated C₁₋₃ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—). In some sub-embodiments, R^{19d} represents —P(O)(R^{19h})₂, wherein each R^{19h} is the same or different and independently represents C₁₋₆ alkyl (e.g., C₁₋₄ alkyl, such as methyl, ethyl, propyl, isopropyl, butyl, sec-butyl, tert-butyl, pentyl, or hexyl), deuterated C₁₋₆ alkyl (e.g., perdeuterated C₁₋₅ alkyl, perdeuterated C₁₋₄ alkyl, or perdeuterated C₁₋₃ alkyl, such as CD₃, CD₃CD₂—, CD₃CD₂CD₂— etc.), or halogenated C₁₋₆ alkyl (e.g., halogenated C₁₋₅ alkyl, or halogenated C₁₋₃ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—). In some sub-embodiments, R^{19d} represents —P(O)(CH₃)₂.

In some embodiments, p19c represents an integer of 0, 1, 2, 3, or 4. In some sub-embodiments, p19c represents an integer of 2.

In some embodiments, PBM represents a structure of Formula (PBM-19-1), Formula (PBM-19-2), Formula (PBM-19-3), Formula (PBM-19-4), or Formula (PBM-19-5):

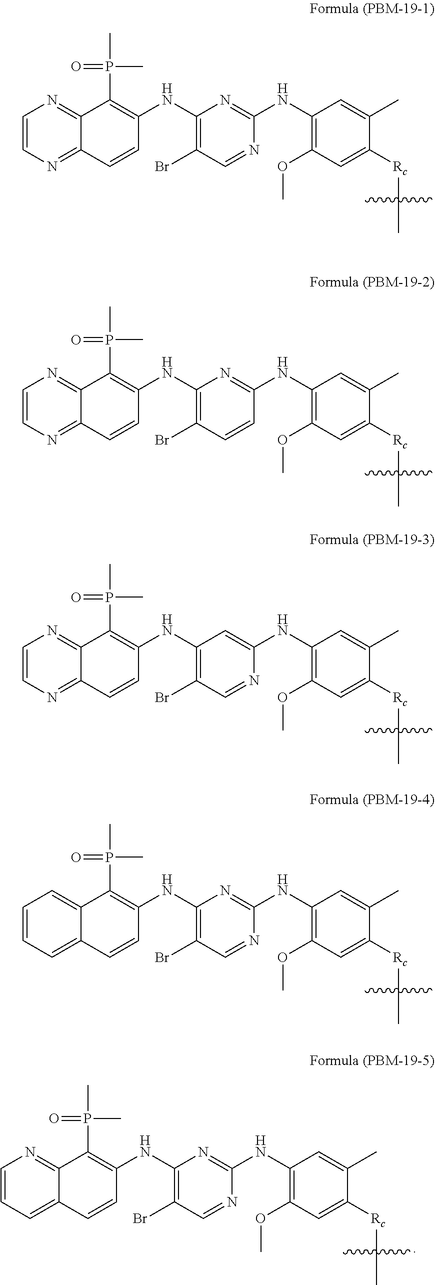

In some embodiments, PBM represents a small molecule ligand of AR.

In some embodiments, PBM represents the structure of Formula (PBM-4):

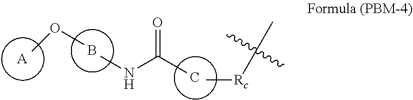

wherein ring A represents C₆₋₁₀ aryl (e.g., phenyl or naphthyl), or 5-to 20-membered heteroaryl (e.g., 5-to 20-membered heteroaryl containing one or two 5-to 7-membered rings and 1 to 4 heteroatoms selected from N, O, and S). Ring A is optionally substituted with one or more (e.g., 1-10, 1-8, or 1-6, such as 1, 2, 3, 4, 5, 6, 7, 8, 9, or 10) R^{4a}, and each R^{4a} is the same or different and each independently represents deuterium, halogen (e.g., fluorine, chlorine, bromine, or iodine), hydroxy, cyano, amino, nitro, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), C₁₋₆ alkoxy (e.g., C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), C₂₋₆ alkenyl (e.g., vinyl, propenyl, or butenyl), or C₂₋₆ alkynyl (e.g., ethynyl, propynyl or butynyl);
ring B represents C₃₋₁₅ cycloalkyl, or 4-to 20-membered heterocyclyl (e.g., 4-to 20-membered heterocyclyl containing 1 to 4 heteroatoms selected from N, O and S), wherein ring B is optionally substituted with one or more (e.g., 1-10, 1-8, or 1-6, such as 1, 2, 3, 4, 5, 6, 7, 8, 9, or 10) R^{4b}, and each R^{4b} is the same or different and each independently represents deuterium, halogen (e.g., fluorine, chlorine, bromine, or iodine), C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), C₁₋₆ alkoxy (e.g., C₁₋₄ alkoxy, such as methoxy, ethoxy, propoxy, isopropoxy, n-butoxy, sec-butoxy, or tert-butoxy), or halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), and
ring C represents 5-to 20-membered heteroaryl (e.g., 5-to 20-membered monocyclic or bicyclic heteroaryl containing 1 to 4 heteroatoms selected from N, O and S), wherein ring C is optionally substituted with one or more (e.g., 1-10, 1-8, or 1-6, such as 1, 2, 3, 4, 5, 6, 7, 8, 9, or 10) R^{4c}, and each R⁴ is the same or different and each independently represents deuterium, halogen (e.g., fluorine, chlorine, bromine, or iodine), hydroxy, cyano, amino, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), C₁₋₆ alkoxy (e.g., C₁₋₄ alkoxy, such as methoxy, ethoxy, propoxy, isopropoxy, n-butoxy, sec-butoxy, or tert-butoxy), or halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—).

In some embodiments, PBM represents the structure of Formula (PBM-4-1A):

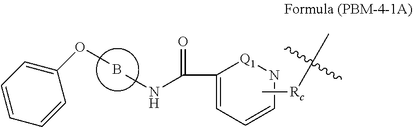

wherein the benzene ring in Formula (PBM-4-1A) is optionally substituted with one or more (e.g., 1-10, 1-8, or 1-6, such as 1, 2, 3, 4, 5, 6, 7, 8, 9, or 10) R^{4a}, and each R^{4a} is the same or different and each independently represents deuterium, halogen (e.g., fluorine, chlorine, bromine, or iodine), hydroxy, cyano, amino, nitro, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), C₁₋₆ alkoxy (e.g., C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), C₂₋₆ alkenyl (e.g., vinyl, propenyl, or butenyl), or C₂₋₆ alkynyl (e.g., ethynyl, propynyl or butynyl);
ring B represents C₃₋₁₅ cycloalkyl or 4-to 20-membered heterocyclyl (e.g., 4-to 20-membered heterocyclyl containing 1 to 4 heteroatoms selected from N, O and S), wherein ring B is optionally substituted with one or more (e.g., 1-10, 1-8, or 1-6, such as 1, 2, 3, 4, 5, 6, 7, 8, 9, or 10) R^{4b}, and each R^{4b} is the same or different and each independently represents deuterium, halogen (e.g., fluorine, chlorine, bromine, or iodine), C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), C₁₋₆ alkoxy (e.g., C₁₋₄ alkoxy, such as methoxy, ethoxy, propoxy, isopropoxy, n-butoxy, sec-butoxy, or tert-butoxy), or halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—);
Q₁ represents N or CH, and
the heteroaryl group containing Q₁ and a nitrogen atom in Formula (PBM-4-1A) is optionally substituted with one or more (e.g., 1-3, such as 1, 2, or 3) R^{4c}, and each R^{4c} is the same or different and each independently represents deuterium, halogen (e.g., fluorine, chlorine, bromine, or iodine), hydroxy, cyano, amino, nitro, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), C₁₋₆ alkoxy (e.g., C₁₋₄ alkoxy, such as methoxy, ethoxy, propoxy, isopropoxy, n-butoxy, sec-butoxy, or tert-butoxy), or halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—).

In some embodiments, ring B in each of Formula (PBM-4) and Formula (PBM-4-1A) each independently represents C₃₋₁₅ cycloalkyl or 4-to 20-membered heterocyclyl (e.g., 4-to 20-membered heterocyclyl containing 1 to 4 heteroatoms selected from N, O and S). Examples of C₃₋₁₅ cycloalkyl include, but are not limited to, cyclopropyl, cyclobutyl, cyclopentyl, cyclopentenyl, cyclohexyl, cyclohexenyl, cycloheptyl, cyclooctyl, decalinyl, octahydropentalenyl, octahydro-1H-indenyl, 2,3-dihydro-1H-indenyl, spiro-cycloalkyl (e.g., C₅₋₁₅ spiro-cycloalkyl), adamantanyl, noradamantanyl, and norbornyl. The optional substituents of C₃₋₁₅ cycloalkyl are optionally selected from the group consisting of halogen (e.g., fluorine, chlorine, bromine, or iodine), hydroxy, mercapto, cyano, amino, nitro, C₁₋₆ alkyl (e.g., C₁₋₄ alkyl, or C₁₋₃ alkyl, such as methyl, ethyl, propyl, isopropyl, butyl, sec-butyl, tert-butyl, pentyl, or hexyl), halogenated C₁₋₆ alkyl (e.g., halogenated C₁₋₄ alkyl, or halogenated C₁₋₃ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), C₁₋₆ alkoxy (e.g., C₁₋₄ alkoxy, such as methoxy, ethoxy, propoxy, isopropoxy, n-butoxy, sec-butoxy, or tert-butoxy), and halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—). Examples of 4-to 20-membered heterocyclyl include, but are not limited to, azetidinyl, oxetanyl, pyrrolidinyl, imidazolidinyl, pyrazolidyl, tetrahydrofuranyl, tetrahydropyranyl, tetrahydrothienyl, tetrahydrothiopyranyl, oxazolidinyl, thiazolidinyl, piperidinyl, piperazinyl, morpholinyl, thiomorpholinyl, dioxacyclohexyl, azacycloheptyl, diazacycloheptanyl (e.g., 1,4-diazacycloheptan-1-yl), azacyclooctyl, diazacyclooctyl, azabicyclo[3.1.1]heptanyl, azabicyclo[2.2.1]heptanyl, azabicyclo[3.2.1]octanyl, azabicyclo[2.2.2]octanyl, diazabicyclo[3.1.1]heptanyl, diazabicyclo[2.2.1]heptanyl, diazabicyclo[3.2.1]octanyl (e.g., 3,8-diazabicyclo[3.2.1]octan-3-yl), and diazabicyclo[2.2.2]octanyl (e.g., 2,5-diazabicyclo[2.2.2]octan-2-yl). The optional substituents of 4-to 20-membered heterocyclyl are optionally selected from the group consisting of halogen (e.g., fluorine, chlorine, bromine, or iodine), hydroxy, mercapto, cyano, amino, nitro, C₁₋₆ alkyl (e.g., C₁₋₄ alkyl, or C₁₋₃ alkyl, such as methyl, ethyl, propyl, isopropyl, butyl, sec-butyl, tert-butyl, pentyl, or hexyl), halogenated C₁₋₆ alkyl (e.g., halogenated C₁₋₄ alkyl, or halogenated C₁₋₃ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), C₁₋₆ alkoxy (e.g., C₁₋₄ alkoxy, such as methoxy, ethoxy, propoxy, isopropoxy, n-butoxy, sec-butoxy, or tert-butoxy), and halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—).

In some embodiments, PBM represents a structure of Formula (PBM-4-1), Formula (PBM-4-2), Formula (PBM-4-3), or Formula (PBM-4-4):

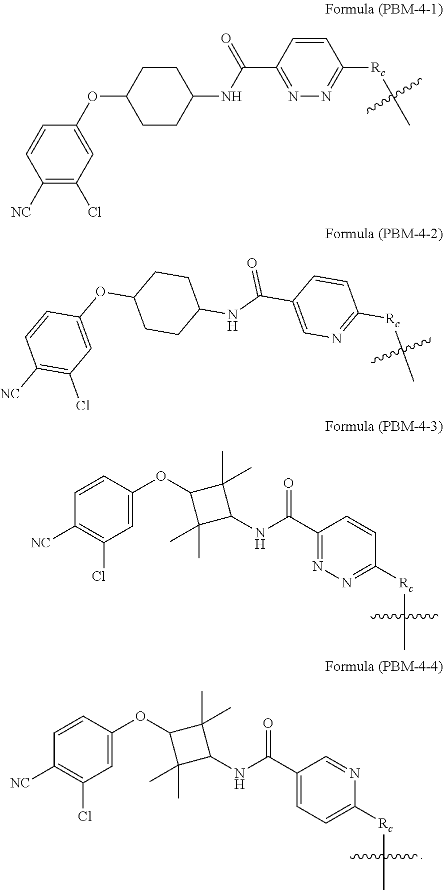

In some embodiments. PBM represents a structure of Formula (PBM-27):

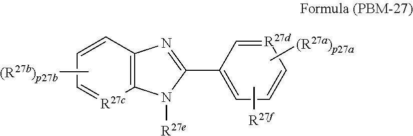

wherein (R^{27a})ₚ₂₇ₐ in Formula (PBM-27) indicates that the 6-membered ring containing R^{27d} to which it is attached is optionally substituted with p27a R^{27a}, wherein each R^{27a} is the same or different and independently represents deuterium, halogen (e.g., fluorine, chlorine, bromine, or iodine), hydroxy, amino, cyano, C₁₋₆ alkoxy (e.g., C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), C₁₋₆ alkyl (e.g., C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), or halogenated C₁₋₆ alkyl (e.g., halogenated C₁₋₄ alkyl, or halogenated C₁₋₃ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—);
p27a represents an integer of 0, 1, 2, or 3;
(R^{27b})_{p27b} represents p27b R^{27b} groups, wherein each R^{27b} is the same or different and independently represents deuterium, halogen (e.g., fluorine, chlorine, bromine, or iodine), hydroxy, amino, cyano, C₁₋₆ alkoxy (e.g., C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), C₁₋₆ alkyl (e.g., C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), halogenated C₁₋₆ alkyl (e.g., halogenated C₁₋₄ alkyl, or halogenated C₁₋₃ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), or optionally substituted C₆₋₁₀ aryl;
p27b represents an integer of 0, 1, 2, or 3;
R^{27c} and R^{27d} each independently represent N or CH;
R^{27e} represents hydrogen or a group having the following structure of Formula (III):

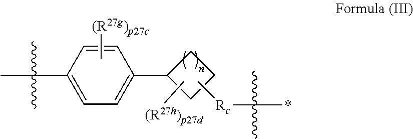

wherein (R^{27g})_{p27c} indicates that the benzene ring to which it is attached is optionally substituted with p27c R^{27g}, wherein each R^{27g} is the same or different and independently represents deuterium, halogen (e.g., fluorine, chlorine, bromine, or iodine), hydroxy, amino, cyano, C₁₋₆ alkoxy (e.g., C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), C₁₋₆ alkyl (e.g., C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), or halogenated C₁₋₆ alkyl (e.g., halogenated C₁₋₄ alkyl, or halogenated C₁₋₃ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—);
p27c represents an integer of 0, 1, 2, 3, or 4;
n represents an integer of 1, 2, 3, 4, or 5;
(R^{27h})ₚ₂₇ₐ represents p27d R^{27h} groups, wherein each R^{27h} is the same or different and independently represents deuterium, halogen (e.g., fluorine, chlorine, bromine, or iodine), hydroxy, amino, cyano, C₁₋₆ alkoxy (e.g., C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), C₁₋₆ alkyl (e.g., C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), or halogenated C₁₋₆ alkyl (e.g., halogenated C₁₋₄ alkyl, or halogenated C₁₋₃ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—);
p27d represents an integer of 0, 1, 2, 3, 4, 5, 6, or 7, and
symbol * indicates the point of attachment to LIN;
R^{27f} represents hydrogen or R_{c};
wherein when R^{27e} represents hydrogen, R^{27f} represents R_{c}; when R^{27f} represents hydrogen, R^{27e} represents the group having the structure of Formula (III).

In some embodiments, R^{27e} represents hydrogen, and R^{27f} represents R_{c}.

In some embodiments, R^{27f} represents hydrogen, and R^{27e} represents the group having the structure of Formula (III):

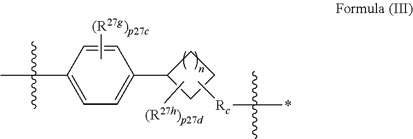

wherein (R^{27g})_{p27c} indicates that the benzene ring to which it is attached is optionally substituted with p27c R^{27g}, wherein each R^{27g} is the same or different and independently represents deuterium, halogen (e.g., fluorine, chlorine, bromine, or iodine), hydroxy, amino, cyano, C₁₋₆ alkoxy (e.g., C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), C₁₋₆ alkyl (e.g., C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), or halogenated C₁₋₆ alkyl (e.g., halogenated C₁₋₄ alkyl, or halogenated C₁₋₃ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—);
p27c represents an integer of 0, 1, 2, 3, or 4;
n represents an integer of 1, 2, 3, 4, or 5;
(R^{27h})ₚ₂₇ₐ indicates that the cycloalkyl to which it is attached is optionally substituted with p27d R^{27h} substituents, wherein each R^{27h} is the same or different and independently represents deuterium, halogen (e.g., fluorine, chlorine, bromine, or iodine), hydroxy, amino, cyano, C₁₋₆ alkoxy (e.g., C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), C₁₋₆ alkyl (e.g., C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), or halogenated C₁₋₆ alkyl (e.g., halogenated C₁₋₄ alkyl, or halogenated C₁₋₃ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—);
p27d represents an integer of 0, 1, 2, 3, 4, 5, 6, or 7, and
symbol * indicates the point of attachment to LIN.

In some embodiments, PBM represents a structure of Formula (PBM-27-A):

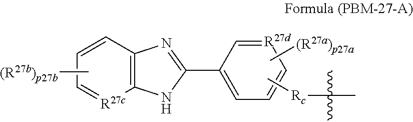

wherein (R^{27a})ₚ₂₇ₐ in Formula (PBM-27-A) indicates that the 6-membered ring containing R^{27d} to which it is attached is optionally substituted with p27a R^{27a}, wherein each R^{27a} is the same or different and independently represents deuterium, halogen (e.g., fluorine, chlorine, bromine, or iodine), hydroxy, amino, cyano, C₁₋₆ alkoxy (e.g., C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), C₁₋₆ alkyl (e.g., C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), or halogenated C₁₋₆ alkyl (e.g., halogenated C₁₋₄ alkyl, or halogenated C₁₋₃ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—);
p27a represents an integer of 0, 1, 2, or 3;
(R^{27b})_{p27b} represents p27b R^{27b} groups, wherein each R^{27b} is the same or different and independently represents deuterium, halogen (e.g., fluorine, chlorine, bromine, or iodine), hydroxy, amino, cyano, C₁₋₆ alkoxy (e.g., C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), C₁₋₆ alkyl (e.g., C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), halogenated C₁₋₆ alkyl (e.g., halogenated C₁₋₄ alkyl, or halogenated C₁₋₃ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), or optionally substituted C₆₋₁₀ aryl;
p27b represents an integer of 0, 1, 2, or 3, and
R^{27c} and R^{27d} each independently represent N or CH.

In some embodiments, R^{27b} in Formula (PBM-27) and Formula (PBM-27-A) each independently represents a C₆₋₁₀ aryl, such as, but not limited to, phenyl or naphthyl, each of which is optionally substituted with one or more (e.g., 1-6, such as 1, 2, 3, 4, 5, or 6) substituents independently selected from the group consisting of deuterium, halogen (e.g., fluorine, chlorine, bromine, or iodine), hydroxy, cyano, amino, nitro, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), C₁₋₆ alkoxy (e.g., C₁₋₄ alkoxy, such as methoxy, ethoxy, propoxy, isopropoxy, n-butoxy, sec-butoxy, or tert-butoxy), or halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—).

In some embodiments, R^{27c} and R^{27d} in Formula (PBM-27-A) represent CH.

In some embodiments, PBM represents a structure of Formula (PBM-27-1):

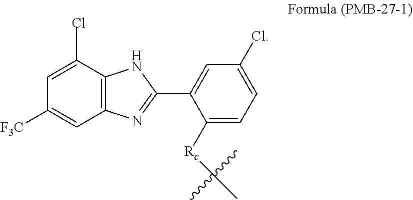

In some embodiments, PBM represents a structure of Formula (PBM-28):

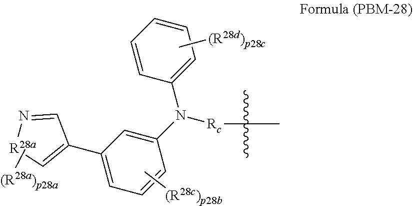

wherein R^{28a} in Formula (PBM-28) represents O, S, or NH;
(R^{28b})ₚ₂₈ₐ indicates that the nitrogen-containing 5-membered ring to which it is attached is optionally substituted with p28a R^{28b}, wherein each R^{28b} is the same or different and independently represents deuterium, halogen (e.g., fluorine, chlorine, bromine, or iodine), hydroxy, amino, cyano, C₁₋₆ alkoxy (e.g., C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), C₁₋₆ alkyl (e.g., C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), or halogenated C₁₋₆ alkyl (e.g., halogenated C₁₋₄ alkyl, or halogenated C₁₋₃ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—);
p28a represents an integer of 0, 1 or 2;
(R^{28c})_{p28b} indicates that the benzene ring to which it is attached is optionally substituted with p28b R^{28c} wherein each R^{28c} is the same or different and independently represents deuterium, halogen (e.g., fluorine, chlorine, bromine, or iodine), hydroxy, amino, cyano, C₁₋₆ alkoxy (e.g., C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), C₁₋₆ alkyl (e.g., C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), or halogenated C₁₋₆ alkyl (e.g., halogenated C₁₋₄ alkyl, or halogenated C₁₋₃ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—);
p28b represents an integer of 0, 1, 2, 3, or 4;
(R^{28d})_{p28c} indicates that the benzene ring to which it is attached is optionally substituted with p28c R^{28d}, wherein each R^{28d} is the same or different and independently represents deuterium, halogen (e.g., fluorine, chlorine, bromine, or iodine), hydroxy, amino, cyano, C₁₋₆ alkoxy (e.g., C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), C₁₋₆ alkyl (e.g., C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), halogenated C₁₋₆ alkyl (e.g., halogenated C₁₋₄ alkyl, or halogenated C₁₋₃ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), or optionally substituted C₃₋₁₅ cycloalkyl, and
p28c represents an integer of 0, 1, 2, 3, 4, or 5.

In some embodiments, R^{28d} in Formula (PBM-28) each independently represents C₃₋₁₅ cycloalkyl. Examples of C₃₋₁₅ cycloalkyl include, but are not limited to, cyclopropyl, cyclobutyl, cyclopentyl, cyclopentenyl, cyclohexyl, cyclohexenyl, cycloheptyl, cyclooctyl, decalinyl, octahydropentalenyl, octahydro-1H-indenyl, 2,3-dihydro-1H-indenyl, spiro-cycloalkyl (e.g., C₅₋₁₅ spiro-cycloalkyl), adamantanyl, noradamantanyl, and norbornyl. The optional substituents of C₃₋₁₅ cycloalkyl are optionally selected from the group consisting of halogen (e.g., fluorine, chlorine, bromine, or iodine), hydroxy, mercapto, cyano, amino, nitro, C₁₋₆ alkyl (e.g., C₁₋₄ alkyl, or C₁₋₃ alkyl, such as methyl, ethyl, propyl, isopropyl, butyl, sec-butyl, tert-butyl, pentyl, or hexyl), halogenated C₁₋₆ alkyl (e.g., halogenated C₁₋₄ alkyl, or halogenated C₁₋₃ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), C₁₋₆ alkoxy (e.g., C₁₋₄ alkoxy, such as methoxy, ethoxy, propoxy, isopropoxy, n-butoxy, sec-butoxy, or tert-butoxy), and halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—).

In some embodiments, R^{28a} in Formula (PBM-28) represents O.

In some embodiments, PBM represents a structure of Formula (PBM-28-1):

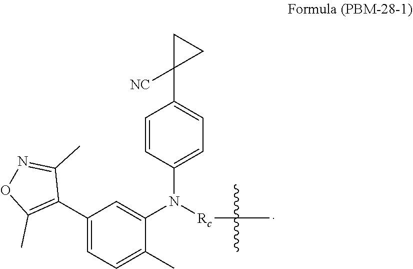

In some embodiments, PBM represents a small molecule ligand of AKT.

In some embodiments, PBM represents a structure of Formula (PBM-27-B):

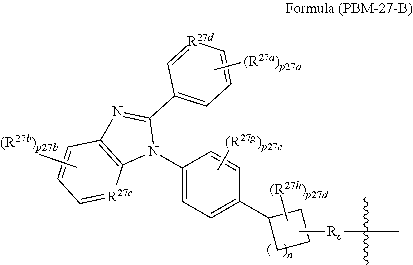

wherein (R^{27a})ₚ₂₇ₐ in Formula (PBM-27-B) indicates that the 6-membered ring containing R^{27d} to which it is attached is optionally substituted with p27a R^{27a}, wherein each R^{27a} is the same or different and independently represents deuterium, halogen (e.g., fluorine, chlorine, bromine, or iodine), hydroxy, amino, cyano, C₁₋₆ alkoxy (e.g., C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), C₁₋₆ alkyl (e.g., C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), or halogenated C₁₋₆ alkyl (e.g., halogenated C₁₋₄ alkyl, or halogenated C₁₋₃ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—);
p27a represents an integer of 0, 1, 2, or 3;
(R^{27b})_{p27b} represents p27b R^{27b} groups, wherein each R^{27b} is the same or different and independently represents deuterium, halogen (e.g., fluorine, chlorine, bromine, or iodine), hydroxy, amino, cyano, C₁₋₆ alkoxy (e.g., C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), C₁₋₆ alkyl (e.g., C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), halogenated C₁₋₆ alkyl (e.g., halogenated C₁₋₄ alkyl, or halogenated C₁₋₃ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), or optionally substituted C₆₋₁₀ aryl;
p27b represents an integer of 0, 1, 2, or 3;
R^{27c} and R^{27d} each independently represent N or CH;
(R^{27g})_{p27c} indicates that the benzene ring to which it is attached is optionally substituted with p27c R^{27g} wherein each R^{27g} is the same or different and independently represents deuterium, halogen (e.g., fluorine, chlorine, bromine, or iodine), hydroxy, amino, cyano, C₁₋₆ alkoxy (e.g., C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), C₁₋₆ alkyl (e.g., C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), or halogenated C₁₋₆ alkyl (e.g., halogenated C₁₋₄ alkyl, or halogenated C₁₋₃ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—);
p27c represents an integer of 0, 1, 2, 3, or 4;
n represents an integer of 1, 2, 3, 4, or 5;
(R^{27h})_{p27d} represents p27d R^{27h} groups, wherein each R^{27h} is the same or different and independently represents deuterium, halogen (e.g., fluorine, chlorine, bromine, or iodine), hydroxy, amino, cyano, C₁₋₆ alkoxy (e.g., C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), C₁₋₆ alkyl (e.g., C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), or halogenated C₁₋₆ alkyl (e.g., halogenated C₁₋₄ alkyl, or halogenated C₁₋₃ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), and
p27d represents an integer of 0, 1, 2, 3, 4, 5, 6, or 7.

In some embodiments, R^{27c} and R^{27d} in Formula (PBM-27-B) represent N.

In some embodiments, PBM represents a structure of Formula (PBM-27-2):

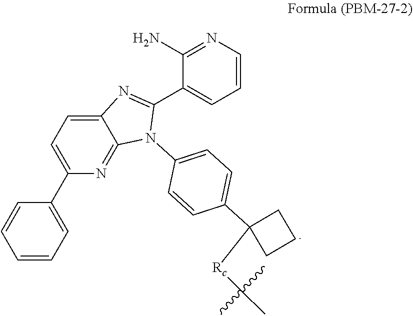

In some embodiments, PBM represents a structure of Formula (PBM-32):

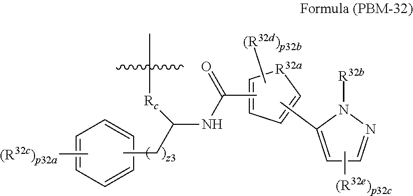

wherein R^{32a} in Formula (PBM-32) represents O, S, or NH;
R^{32b} represents hydrogen, C₁₋₆ alkyl (e.g., C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), or halogenated C₁₋₆ alkyl (e.g., halogenated C₁₋₄ alkyl, or halogenated C₁₋₃ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—);
Z3 represents an integer of 1, 2, or 3;
(R^{32c})ₚ₃₂ₐ indicates that the benzene ring to which it is attached is optionally substituted with p32a R^{32c} substituents, wherein each R^{32c} is the same or different and independently represents deuterium, halogen (e.g., fluorine, chlorine, bromine, or iodine), hydroxy, amino, cyano, C₁₋₆ alkoxy (e.g., C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), C₁₋₆ alkyl (e.g., C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), or halogenated C₁₋₆ alkyl (e.g., halogenated C₁₋₄ alkyl, or halogenated C₁₋₃ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—);
p32a represents an integer of 0, 1, 2, 3, 4, or 5;
(R^{32d})_{p32b} indicates that the 5-membered ring containing R^{32a} to which it is attached is optionally substituted with p32b R^{32d} substituents, wherein each R^{32d} is the same or different and independently represents deuterium, halogen (e.g., fluorine, chlorine, bromine, or iodine), hydroxy, amino, cyano, C₁₋₆ alkoxy (e.g., C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), C₁₋₆ alkyl (e.g., C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), or halogenated C₁₋₆ alkyl (e.g., halogenated C₁₋₄ alkyl, or halogenated C₁₋₃ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—);
p32b represents an integer of 0, 1 or 2;
(R^{32e})_{p32c} indicates that the pyrazole ring to which it is attached is optionally substituted by p32c R^{32e} substituents, wherein each R^{32e} is the same or different and independently represents deuterium, halogen (e.g., fluorine, chlorine, bromine, or iodine), hydroxy, amino, cyano, C₁₋₆ alkoxy (e.g., C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), C₁₋₆ alkyl (e.g., C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), or halogenated C₁₋₆ alkyl (e.g., halogenated C₁₋₄ alkyl, or halogenated C₁₋₃ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), and
p32c represents an integer of 0, 1 or 2.

In some embodiments, PBM represents a structure of Formula (PBM-32-1) or Formula (PBM-32-2):

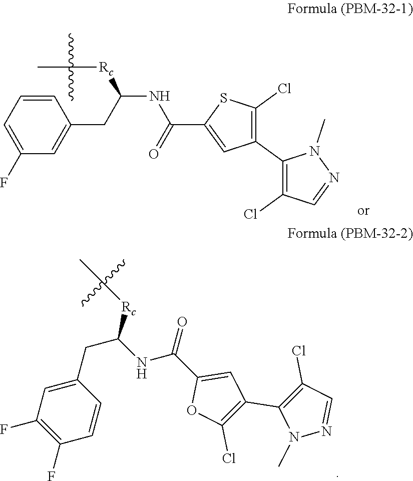

In some embodiments, PBM represents a small molecule ligand of BTK.

In some embodiments, PBM represents a structure of Formula (PBM-5):

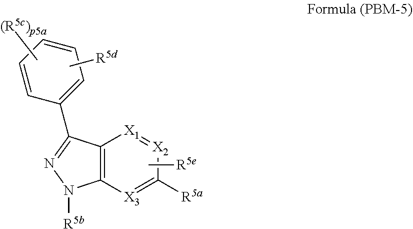

wherein X₁ in Formula (PBM-5) represents N or CR^{5f}, where R^{5f} represents hydrogen, deuterium, or amino, and X₂ represents N or CR^{5g}, where R^{5g} represents hydrogen or R_{c}, and X₃ represents CH or N;
R^{5a} and R^{5b} each independently represent hydrogen or R_{c};
(R^{5c})ₚ₅ₐ indicates that the benzene ring to which it is attached is substituted with p5a R^{5c}, with each R^{5c} being the same or different and independently representing —O-aryl, —O-heteroaryl, 4-to 20-membered heterocyclyl, —C₁₋₃ alkylene-NHC(O)-heteroaryl, —C₁₋₃ alkylene-C(O)NH-heteroaryl, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), deuterium, deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), halogen (e.g., fluorine, chlorine, bromine, or iodine), or halogenated C₁₋₆ alkyl (e.g., halogenated C₁₋₄ alkyl, or halogenated C₁₋₃ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), wherein the 4-to 20-membered heterocyclyl, aryl and heteroaryl are each independently optionally substituted with one or more (e.g., 1-5, such as 1, 2, 3, 4, or 5) R^{5h}, and each R^{5h} is the same or different and each independently represents deuterium, halogen (e.g., fluorine, chlorine, bromine, or iodine), hydroxy, cyano, amino, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), halogenated C₁₋₆ alkyl (e.g., halogenated C₁₋₄ alkyl, or halogenated C₁₋₃ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), C₁₋₆ alkoxy (e.g., C₁₋₄ alkoxy, or C₁₋₃ alkoxy, such as methoxy, ethoxy, propoxy, isopropoxy, n-butoxy, sec-butoxy, or tert-butoxy), or halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—);
p5a represents an integer of 1, 2, 3, or 4;
R^{5d} represents R_{c}, hydrogen, halogen (e.g., fluorine, chlorine, bromine, or iodine), C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), halogenated C₁₋₆ alkyl (e.g., halogenated C₁₋₄ alkyl, or halogenated C₁₋₃ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), or C₁₋₆ alkoxy (e.g., C₁₋₄ alkoxy, or C₁₋₃ alkoxy, such as methoxy, ethoxy, propoxy, isopropoxy, n-butoxy, sec-butoxy, or tert-butoxy);
R^{5e} represents hydrogen, halogen (e.g., fluorine, chlorine, bromine, or iodine), cyano, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), halogenated C₁₋₆ alkyl (e.g., halogenated C₁₋₄ alkyl, or halogenated C₁₋₃ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), or amino;
wherein R^{5g}, R^{5a}, R^{5b}, and R^{5d} are not simultaneously hydrogen, and only one of R^{5g}, R^{5a}, R^{5b}, and R^{5d} represents R_{c}, wherein
   when R^{5a} represents R_{c}, X₂ represents CH or N, and R^{5b} is hydrogen, and R^{5d} represents hydrogen, halogen, C₁₋₆ alkyl, halogenated C₁₋₆ alkyl, deuterated C₁₋₆ alkyl, halogenated C₁₋₆ alkoxy, or C₁₋₆ alkoxy;
   when X₂ represents CR^{5g}, where R^{5g} represents R_{c}, R^{5a} and R^{5b} are hydrogen, and R^{5d} represents hydrogen, halogen, C₁₋₆ alkyl, halogenated C₁₋₆ alkyl, deuterated C₁₋₆ alkyl, halogenated C₁₋₆ alkoxy, or C₁₋₆ alkoxy;
   when R^{5b} represents R_{c}, X₂ represents N or CH, and R^{5a} is hydrogen, and R^{5d} represents hydrogen, halogen, C₁₋₆ alkyl, halogenated C₁₋₆ alkyl, deuterated C₁₋₆ alkyl, halogenated C₁₋₆ alkoxy, or C₁₋₆ alkoxy, or
   when R^{5d} represents R_{c}, X₂ represents N or CH, and R^{5a} and R^{5b} are hydrogen.

As used herein, one of R^{5g}, R^{5a}, R^{5b}, and R^{5d} in Formula (PBM-5) represents R_{c}, and the remaining ones are not R_{c}.

In some embodiments, R^{5c} in Formula (PBM-5) represents —O-aryl, —O-heteroaryl, —C₁₋₃ alkylene-NHC(O)-heteroaryl, or —C₁₋₃ alkylene-C(O)NH-heteroaryl, wherein the aryl may be optionally substituted C₆₋₁₀ aryl, including but not limited to e.g., phenyl or naphthyl, and the heteroaryl may be optionally substituted5-to 14-membered monocyclic, bicyclic, or polycyclic aromatic ring group containing one or more (e.g., from 1 to 6, or from 1 to 5, or from 1 to 4, or from 1 to 3) heteroatoms selected from the group consisting of oxygen, nitrogen, and sulfur. In a sub-embodiment, R^{5c} represents —O-phenyl or —O-naphthyl, wherein the phenyl and naphthyl are each optionally substituted with one or more (e.g., 1-5, such as 1, 2, 3, 4, or 5) R^{5h}, and each R^{5h} is the same or different and each independently represents deuterium, halogen (e.g., fluorine, chlorine, bromine, or iodine), hydroxy, cyano, amino, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), halogenated C₁₋₆ alkyl (e.g., halogenated C₁₋₄ alkyl, or halogenated C₁₋₃ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), C₁₋₆ alkoxy (e.g., C₁₋₄ alkoxy, or C₁₋₃ alkoxy, such as methoxy, ethoxy, propoxy, isopropoxy, n-butoxy, sec-butoxy, or tert-butoxy), or halogenated C₁₋₆ alkoxy (e.g., halogenated C₁₋₄ alkoxy, or halogenated C₁₋₃ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—). In a sub-embodiment, R^{5c} represents —O-heteroaryl, —C₁₋₃ alkylene-NHC(O)-heteroaryl, or —C₁₋₃ alkylene-C(O)NH-heteroaryl, wherein the heteroaryl is optionally substituted with one or more (e.g., 1-5, such as 1, 2, 3, 4, or 5) R^{5h}, wherein R^{5h} is the same or different and each independently represents deuterium, halogen (e.g., fluorine, chlorine, bromine, or iodine), hydroxy, cyano, amino, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), halogenated C₁₋₆ alkyl (e.g., halogenated C₁₋₄ alkyl, or halogenated C₁₋₃ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), C₁₋₆ alkoxy (e.g., C₁₋₄ alkoxy, or C₁₋₃ alkoxy, such as methoxy, ethoxy, propoxy, isopropoxy, n-butoxy, sec-butoxy, or tert-butoxy), or halogenated C₁₋₆ alkoxy (e.g., halogenated C₁₋₄ alkoxy, or halogenated C₁₋₃ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—). In a sub-embodiment, examples of heteroaryl include, but are not limited to, furanyl, oxazolyl, isoxazolyl, oxadiazolyl, thienyl, thiazolyl, isothiazolyl, thiadiazolyl, pyrrolyl, imidazolyl, pyrazolyl, triazolyl, pyridyl, pyrimidinyl, pyridazinyl, pyrazinyl, indolyl, isoindolyl, benzofuranyl, isobenzofuranyl, benzothienyl, indazolyl, benzimidazolyl, benzoxazolyl, benzisoxazolyl, benzothiazolyl, benzisothiazolyl, benzotriazolyl, benzo[2,1,3]oxadiazolyl, benzo[2,1,3]thiadiazolyl, benzo[1,2,3]thiadiazolyl, quinolinyl, isoquinolinyl, naphthyridinyl, cinnolinyl, quinazolinyl, quinoxalinyl, phthalazinyl, pyrazolo[1,5-a]pyridyl, pyrazolo[1,5-alpyrimidinyl, imidazo[1,2-a]pyridyl, 1H-pyrrolo[3,2-b]pyridyl, 1H-pyrrolo[2,3-b]pyridyl, pyrrolo[2,1-b]thiazolyl, and imidazo[2,1-b]thiazolyl.

In some embodiments, R^{5c} in Formula (PBM-5) represents 4-to 20-membered (e.g., 4-to 15-membered, 4-to 12-membered, 4-to 10-membered, 4-to 7-membered, or 4-to 6-membered)monocyclic, bicyclic, or polycyclic heterocyclyl, wherein the 4-to 20-membered monocyclic, bicyclic, or polycyclic heterocyclyl may be optionally substituted. In a sub-embodiment, R^{5c} represents 4-to 20-membered monocyclic, bicyclic, or polycyclic heterocyclyl, which is optionally substituted with one or more (e.g., 1-5, such as 1, 2, 3, 4, or 5) R^{5h}, and each R^{5h} is the same or different and each independently represents deuterium, halogen (e.g., fluorine, chlorine, bromine, or iodine), hydroxy, cyano, amino, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), halogenated C₁₋₆ alkyl (e.g., halogenated C₁₋₄ alkyl, or halogenated C₁₋₃ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), C₁₋₆ alkoxy (e.g., C₁₋₄ alkoxy, or C₁₋₃ alkoxy, such as methoxy, ethoxy, propoxy, isopropoxy, n-butoxy, sec-butoxy, or tert-butoxy), or halogenated C₁₋₆ alkoxy (e.g., halogenated C₁₋₄ alkoxy, or halogenated C₁₋₃ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—). In a sub-embodiment, examples of 4-to 20-membered monocyclic, bicyclic, or polycyclic heterocyclyl include, but are not limited to, azetidinyl, oxetanyl, pyrrolidinyl, imidazolidinyl, pyrazolidyl, tetrahydrofuranyl, tetrahydropyranyl, tetrahydrothienyl, tetrahydrothiopyranyl, oxazolidinyl, thiazolidinyl, piperidinyl, piperazinyl, morpholinyl, thiomorpholinyl, dioxacyclohexyl, azacycloheptyl, diazacycloheptanyl (e.g., 1,4-diazacycloheptan-1-yl), azacyclooctyl, diazacyclooctyl, azabicyclo[3.1.1]heptanyl, azabicyclo[2.2.1]heptanyl, azabicyclo[3.2.1]octanyl, azabicyclo[2.2.2]octanyl, diazabicyclo[3.1.1]heptanyl, diazabicyclo[2.2.1]heptanyl, diazabicyclo[3.2.1]octanyl ((e.g., 3,8-diazabicyclo[3.2.1]octan-3-yl), and diazabicyclo[2.2.2]octanyl (e.g., 2,5-diazabicyclo[2.2.2]octan-2-yl).

In some embodiments, R^{5b} represents R_{c}; R^{5a} is hydrogen; X₂ represents N or CH; and R^{5d} represents hydrogen, halogen, C₁₋₆ alkyl, halogenated C₁₋₆ alkyl, deuterated C₁₋₆ alkyl, halogenated C₁₋₆ alkoxy, or C₁₋₆ alkoxy.

In some embodiments, R^{5b} represents R_{c}; R^{5a} is hydrogen; R^{5d} represents hydrogen, halogen, C₁₋₆ alkyl, halogenated C₁₋₆ alkyl, deuterated C₁₋₆ alkyl, halogenated C₁₋₆ alkoxy, or C₁₋₆ alkoxy; and X₂ represents N; X₁ represents CR^{5f}, wherein R^{5f} is hydrogen or amino; and X₃ represents N.

In some embodiments, R^{5b} represents R_{c}; R^{5a} is hydrogen; R^{5d} represents hydrogen, halogen, C₁₋₆ alkyl, halogenated C₁₋₆ alkyl, deuterated C₁₋₆ alkyl, halogenated C₁₋₆ alkoxy, or C₁₋₆ alkoxy; and X₂ represents N; X₁ represents CR^{5f}, wherein R^{5f} is hydrogen or amino; and X₃ represents CH.

In some embodiments, R^{5b} represents R_{c}; R^{5a} is hydrogen; R^{5d} represents hydrogen, halogen, C₁₋₆ alkyl, halogenated C₁₋₆ alkyl, deuterated C₁₋₆ alkyl, halogenated C₁₋₆ alkoxy, or C₁₋₆ alkoxy; and X₂ represents N; X₁ represents N; and X₃ represents CH.

In some embodiments, R^{5b} represents R_{c}; R^{5a} is hydrogen; R^{5d} represents hydrogen, halogen, C₁₋₆ alkyl, halogenated C₁₋₆ alkyl, deuterated C₁₋₆ alkyl, halogenated C₁₋₆ alkoxy, or C₁₋₆ alkoxy; and X₂ represents N; X₁ represents N; and X₃ represents N.

In some embodiments, R^{5b} represents R_{c}; R^{5a} is hydrogen; R^{5d} represents hydrogen, halogen, C₁₋₆ alkyl, halogenated C₁₋₆ alkyl, deuterated C₁₋₆ alkyl, halogenated C₁₋₆ alkoxy, or C₁₋₆ alkoxy; and X₂ represents CH; X₁ represents CR^{5f}, wherein R^{5f} is hydrogen or amino; and X₃ represents N.

In some embodiments, R^{5b} represents R_{c}; R^{5a} is hydrogen; R^{5d} represents hydrogen, halogen, C₁₋₆ alkyl, halogenated C₁₋₆ alkyl, deuterated C₁₋₆ alkyl, halogenated C₁₋₆ alkoxy, or C₁₋₆ alkoxy; and X₂ represents CH, X₁ represents CR^{5f}, wherein R^{5f} is hydrogen or amino; and X₃ represents CH.

In some embodiments, R^{5b} represents R_{c}; R^{5a} is hydrogen; R^{5d} represents hydrogen, halogen, C₁₋₆ alkyl, halogenated C₁₋₆ alkyl, deuterated C₁₋₆ alkyl, halogenated C₁₋₆ alkoxy, or C₁₋₆ alkoxy; and X₂ represents CH; X₁ represents N; and X₃ represents CH.

In some embodiments, R^{5b} represents R_{c}; R^{5a} is hydrogen; R^{5d} represents hydrogen, halogen, C₁₋₆ alkyl, halogenated C₁₋₆ alkyl, deuterated C₁₋₆ alkyl, halogenated C₁₋₆ alkoxy, or C₁₋₆ alkoxy; and X₂ represents CH; X₁ represents N; and X₃ represents N.

In some embodiments, PBM represents a structure of Formula (PBM-5-1A):

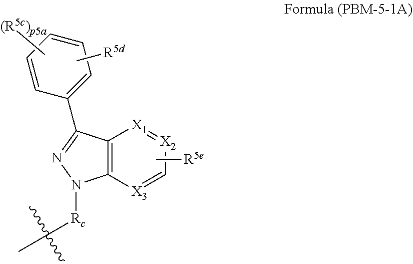

wherein X₁ in Formula (PBM-5-1A) represents N or CR^{5f}, where R^{5f} is hydrogen or amino, and X₂ represents N or CH, and X₃ represents CH or N;
(R^{5c})ₚ₅ₐ in Formula (PBM-5-1A) indicates that the benzene ring to which it is attached is substituted with p5a R^{5c}, and each R^{5c} is the same or different and independently represents —O-aryl, —O-heteroaryl, 4-to 20-membered heterocyclyl, —C₁₋₃ alkylene-NHC(O)-heteroaryl, or —C₁₋₃ alkylene-C(O)NH-heteroaryl, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), deuterium, deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), halogen (e.g., fluorine, chlorine, bromine, or iodine), or halogenated C₁₋₆ alkyl (e.g., halogenated C₁₋₄ alkyl, or halogenated C₁₋₃ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), wherein the 4-to 20-membered heterocyclyl, aryl and heteroaryl are each independently optionally substituted with one or more (e.g., 1-5, such as 1, 2, 3, 4, or 5) R^{5h}, and each R^{5h} is the same or different and independently represents deuterium, halogen (e.g., fluorine, chlorine, bromine, or iodine), hydroxy, cyano, amino, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), halogenated C₁₋₆ alkyl (e.g., halogenated C₁₋₄ alkyl, or halogenated C₁₋₃ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), C₁₋₆ alkoxy (e.g., C₁₋₄ alkoxy, or C₁₋₃ alkoxy, such as methoxy, ethoxy, propoxy, isopropoxy, n-butoxy, sec-butoxy, or tert-butoxy), or halogenated C₁₋₆ alkoxy (e.g., halogenated C₁₋₄ alkoxy, or halogenated C₁₋₃ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—);
p5a represents an integer of 1, 2, 3, or 4;
R^{5d} represents hydrogen, halogen (e.g., fluorine, chlorine, bromine, or iodine), C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), halogenated C₁₋₆ alkyl (e.g., halogenated C₁₋₄ alkyl, or halogenated C₁₋₃ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), or C₁₋₆ alkoxy (e.g., C₁₋₄ alkoxy, or C₁₋₃ alkoxy, such as methoxy, ethoxy, propoxy, isopropoxy, n-butoxy, sec-butoxy, or tert-butoxy); e.g., R^{5d} represents hydrogen or methyl, and
R^{5e} represents hydrogen, halogen (e.g., fluorine, chlorine, bromine, or iodine), cyano, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), halogenated C₁₋₆ alkyl (e.g., halogenated C₁₋₄ alkyl, or halogenated C₁₋₃ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), or amino.

In some embodiments, R^{5c} in Formula (PBM-5-1A) represents-O-aryl, —O-heteroaryl, —C₁₋₃ alkylene-NHC(O)-heteroaryl, or—C₁₋₃ alkylene-C(O)NH-heteroaryl, wherein the aryl may be optionally substituted C₆₋₁₀ aryl, including but not limited to e.g., phenyl or naphthyl, and the heteroaryl may be optionally substituted 5-to 14-membered monocyclic or bicyclic aromatic ring group containing one or more (e.g., from 1 to 6, or from 1 to 5, or from 1 to 4, or from 1 to 3) heteroatoms selected from the group consisting of oxygen, nitrogen, and sulfur. In a sub-embodiment, R^{5c} represents-O-phenyl or—O-naphthyl, wherein the phenyl and naphthyl are each optionally substituted with one or more (e.g., 1-5, such as 1, 2, 3, 4, or 5) R^{5h}, and each R^{5h} is the same or different and each independently represents deuterium, halogen (e.g., fluorine, chlorine, bromine, or iodine), hydroxy, cyano, amino, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), halogenated C₁₋₆ alkyl (e.g., halogenated C₁₋₄ alkyl, or halogenated C₁₋₃ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), C₁₋₆ alkoxy (e.g., C₁₋₄ alkoxy, or C₁₋₃ alkoxy, such as methoxy, ethoxy, propoxy, isopropoxy, n-butoxy, sec-butoxy, or tert-butoxy), or halogenated C₁₋₆ alkoxy (e.g., halogenated C₁₋₄ alkoxy, or halogenated C₁₋₃ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—). In a sub-embodiment, R^{5c} represents-O-heteroaryl, —C₁₋₃ alkylene-NHC(O)-heteroaryl, or—C₁₋₃ alkylene-C(O)NH-heteroaryl, wherein the heteroaryl is optionally substituted with one or more (e.g., 1-5, such as 1, 2, 3, 4, or 5) R^{5h}, and each R^{5h} is the same or different and each independently represents deuterium, halogen (e.g., fluorine, chlorine, bromine, or iodine), hydroxy, cyano, amino, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), halogenated C₁₋₆ alkyl (e.g., halogenated C₁₋₄ alkyl, or halogenated C₁₋₃ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), C₁₋₆ alkoxy (e.g., C₁₋₄ alkoxy, or C₁₋₃ alkoxy, such as methoxy, ethoxy, propoxy, isopropoxy, n-butoxy, sec-butoxy, or tert-butoxy), or halogenated C₁₋₆ alkoxy (e.g., halogenated C₁₋₄ alkoxy, or halogenated C₁₋₃ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—). In a sub-embodiment, examples of heteroaryl include, but are not limited to, furanyl, oxazolyl, isoxazolyl, oxadiazolyl, thienyl, thiazolyl, isothiazolyl, thiadiazolyl, pyrrolyl, imidazolyl, pyrazolyl, triazolyl, pyridyl, pyrimidinyl, pyridazinyl, pyrazinyl, indolyl, isoindolyl, benzofuranyl, isobenzofuranyl, benzothienyl, indazolyl, benzimidazolyl, benzoxazolyl, benzisoxazolyl, benzothiazolyl, benzisothiazolyl, benzotriazolyl, benzo[2,1,3]oxadiazolyl, benzo[2,1,3]thiadiazolyl, benzo[1,2,3]thiadiazolyl, quinolinyl, isoquinolinyl, naphthyridinyl, cinnolinyl, quinazolinyl, quinoxalinyl, phthalazinyl, pyrazolo[1,5-a]pyridyl, pyrazolo[1,5-alpyrimidinyl, imidazo[1,2-a]pyridyl, 1H-pyrrolo[3,2-b]pyridyl, 1H-pyrrolo[2,3-b]pyridyl, pyrrolo[2,1-b]thiazolyl, and imidazo[2,1-b]thiazolyl.

In some embodiments, R^{5c} in Formula (PBM-5-1A) represents 4-to 20-membered (e.g., 4-to 15-membered, 4-to 12-membered, 4-to 10-membered, 4-to 7-membered, or 4-to 6-membered)monocyclic, bicyclic, or polycyclic heterocyclyl, wherein the 4-to 20-membered monocyclic, bicyclic, or polycyclic heterocyclyl may be optionally substituted. In a sub-embodiment, R^{5c} represents 4-to 20-membered monocyclic, bicyclic, or polycyclic heterocyclyl, which is optionally substituted with one or more (e.g., 1-5, such as 1, 2, 3, 4, or 5) R^{5h}, and each R^{5h} is the same or different and each independently represents deuterium, halogen (e.g., fluorine, chlorine, bromine, or iodine), hydroxy, cyano, amino, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), halogenated C₁₋₆ alkyl (e.g., halogenated C₁₋₄ alkyl, or halogenated C₁₋₃ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), C₁₋₆ alkoxy (e.g., C₁₋₄ alkoxy, or C₁₋₃ alkoxy, such as methoxy, ethoxy, propoxy, isopropoxy, n-butoxy, sec-butoxy, or tert-butoxy), or halogenated C₁₋₆ alkoxy (e.g., halogenated C₁₋₄ alkoxy, or halogenated C₁₋₃ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—). In a sub-embodiment, examples of 4-to 20-membered monocyclic, bicyclic, or polycyclic heterocyclyl include, but are not limited to, azetidinyl, oxetanyl, pyrrolidinyl, imidazolidinyl, pyrazolidyl, tetrahydrofuranyl, tetrahydropyranyl, tetrahydrothienyl, tetrahydrothiopyranyl, oxazolidinyl, thiazolidinyl, piperidinyl, piperazinyl, morpholinyl, thiomorpholinyl, dioxacyclohexyl, azacycloheptyl, diazacycloheptanyl (e.g., 1,4-diazacycloheptan-1-yl), azacyclooctyl, diazacyclooctyl, azabicyclo[3.1.1]heptanyl, azabicyclo[2.2.1]heptanyl, azabicyclo[3.2.1]octanyl, azabicyclo[2.2.2]octanyl, diazabicyclo[3.1.1]heptanyl, diazabicyclo[2.2.1]heptanyl, diazabicyclo[3.2.1]octanyl (e.g., 3,8-diazabicyclo[3.2.1]octan-3-yl), and diazabicyclo[2.2.2]octanyl (e.g., 2,5-diazabicyclo[2.2.2]octan-2-yl).

In some embodiments, X₁ in Formula (PBM-5-1A) represents N; X₂ represents N; and X₃ represents CH.

In some embodiments, X₁ in Formula (PBM-5-1A) represents N; X₂ represents N; and X₃ represents N.

In some embodiments, X₁ in Formula (PBM-5-1A) represents N; X₂ represents CH; and X₃ represents CH.

In some embodiments, X₁ in Formula (PBM-5-1A) represents N; X₂ represents CH; and X₃ represents N.

In some embodiments, X₁ in Formula (PBM-5-1A) represents CR^{5f}, wherein R^{5f} is hydrogen or amino; X₂ represents N; and X₃ represents N.

In some embodiments, X₁ in Formula (PBM-5-1A) represents CR^{5f}, wherein R^{5f} is hydrogen or amino; X₂ represents N; and X₃ represents CH.

In some embodiments, X₁ in Formula (PBM-5-1A) represents CR^{5f}, wherein R^{5f} is hydrogen or amino; X₂ represents CH; and X₃ represents N.

In some embodiments, X₁ in Formula (PBM-5-1A) represents CR^{5f}, wherein R^{5f} is hydrogen or amino; X₂ represents CH; and X₃ represents CH.

In some embodiments, PBM represents the structure of Formula (PBM-5-1):

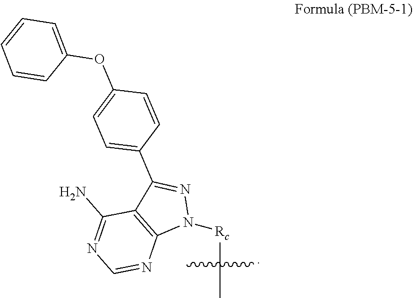

In some embodiments, PBM represents a small molecule ligand of CDK4/6.

In some embodiments, PBM represents a structure of Formula (PBM-6-1A), Formula (PBM-6-1B), Formula (PBM-6-1C), or Formula (PBM-6-1D):

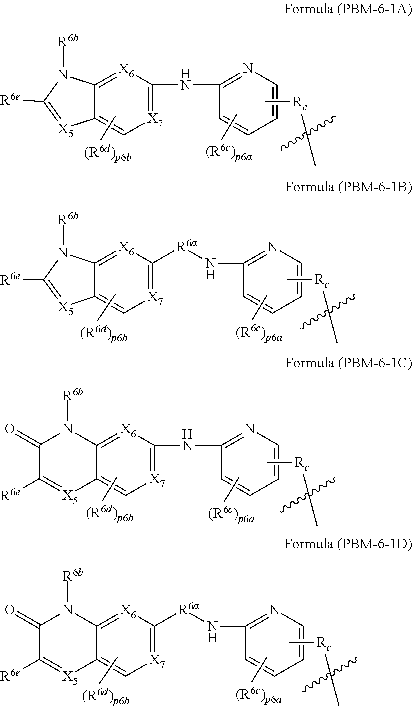

wherein each R^{6e} independently represents deuterium, hydrogen, halogen (e.g., fluorine, chlorine, bromine, or iodine), C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), halogenated C₁₋₆ alkyl (e.g., halogenated C₁₋₄ alkyl, or halogenated C₁₋₃ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), —C(O)—C₁₋₆ alkyl (e.g., —C(O)—C₁₋₅ alkyl, or—C(O)—C₁₋₃ alkyl, such as —C(O)—CH₃, —C(O)—CH₂CH₃), —C(O)-deuterated C₁₋₆ alkyl (e.g., —C(O)-deuterated C₁₋₅ alkyl, or —C(O)-deuterated C₁₋₃ alkyl, such as —C(O)—CD₃), or —C(O)NR^{6f}R^{6g}, wherein R^{6f}, R^{6g} are the same or different and independently represent hydrogen, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), or deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), and R^{6f} and R^{6g} are not simultaneously hydrogen;

each X₅ independently represents N or CR^{6h}, wherein R^{6h} represents hydrogen, deuterium, halogen (e.g., fluorine, chlorine, bromine, or iodine), C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), or halogenated C₁₋₆ alkyl (e.g., halogenated C₁₋₄ alkyl, or halogenated C₁₋₃ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—);
X₆ and X₇ each independently represent CH or N;
R^{6a} in each of Formula (PBM-6-1B) and Formula (PBM-6-1D) each independently represents optionally substituted heteroarylene (e.g., optionally substituted 5-to 20-membered monocyclic, bicyclic, or polycyclic aromatic ring group containing one or more (e.g., from 1 to 6, or from 1 to 5, or from 1 to 4, or from 1 to 3) heteroatoms independently selected from the group consisting of oxygen, nitrogen, and sulfur), wherein the heteroarylene is optionally substituted with 1-2 substituents each independently selected from the group consisting of halogen (e.g., fluorine, chlorine, bromine, or iodine), hydroxy, cyano, amino, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), C₁₋₆ alkoxy (e.g., C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), and any combination thereof;
each Rob independently represents hydrogen, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), or optionally substituted C₃₋₁₂ cycloalkyl (e.g., cyclopropyl, cyclobutyl, cyclopentyl, cyclopentenyl, cyclohexyl, cyclohexenyl, or cycloheptyl, which are optionally substituted with a substituent selected from the group consisting of e.g., halogen, C₁₋₆ alkyl, halogenated C₁₋₆ alkyl, or deuterated C₁₋₆ alkyl);
each (R^{6c})ₚ₆ₐ independently indicates that the pyridine ring to which it is attached is optionally substituted with p6a R^{6c}, wherein each R^{6c} is the same or different and independently represents deuterium, halogen (e.g., fluorine, chlorine, bromine, or iodine), C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), C₁₋₆ alkoxy (e.g., C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), or halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), and poa represents an integer of 0, 1, 2, or 3, and
each (R^{6d})_{p6b} independently indicates that the nitrogen-containing bicyclic heteroaryl ring to which it is attached is optionally substituted with p6b Rod, wherein each Rod is the same or different and independently represents deuterium, halogen (e.g., fluorine, chlorine, bromine, or iodine), C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), C₁₋₆ alkoxy (e.g., C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), or halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), and p6b represents an integer of 0 or 1.

In some embodiments, R^{6a} in each of Formula (PBM-6-1B) and Formula (PBM-6-1D) independently represents optionally substituted heteroarylene, e.g., optionally substituted 5-to 14-membered monocyclic, bicyclic, or polycyclic heteroarylene group containing one or more (e.g., from 1 to 6, or from 1 to 5, or from 1 to 4, or from 1 to 3) heteroatoms independently selected from the group consisting of oxygen, nitrogen, and sulfur. Examples of heteroarylene include, but are not limited to, furanylene, oxazolylene, isoxazolylene, oxadiazolylene, thienylene, thiazolylene, isothiazolylene, thiadiazolylene, pyrrolylene, imidazolylene, pyrazolylene, triazolylene, pyridylene, pyrimidinylene, pyridazinylene, pyrazinylene, indolylene, isoindolylene, benzofuranylene, isobenzofuranylene, benzothienylene, indazolylene, benzimidazolylene, benzoxazolylene, benzisoxazolylene, benzothiazolylene, benzisothiazolylene, benzotriazolylene, benzo[2,1,3]oxadiazolylene, benzo[2,1,3]thiadiazolylene, benzo[1,2,3]thiadiazolylene, quinolinylene, isoquinolinylene, naphthyridinylene, cinnolinylene, quinazolinylene, quinoxalinylene, phthalazinylene, pyrazolo[1,5-alpyridylene, pyrazolo[1,5-a]pyrimidinylene, imidazo[1,2-a]pyridylene, 1H-pyrrolo[3,2-b]pyridylene, 1H-pyrrolo[2,3-b]pyridylene, 4H-furo[3,2-b]pyrrolylene, pyrrolo[2,1-b]thiazolylene, and imidazo[2,1-b]thiazolylene. Heteroarylene is optionally substituted with one or more (e.g., 1-4, such as 1, 2, 3, or 4) substituents selected from the group consisting of: halogen (e.g., fluorine, chlorine, bromine, or iodine), hydroxy, cyano, amino, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), C₁₋₆ alkoxy (e.g., C₁₋₄ alkoxy, such as methoxy, ethoxy, propoxy, isopropoxy, n-butoxy, sec-butoxy, or tert-butoxy), or halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—).

In some embodiments, PBM represents the structure of Formula (PBM-6-1), Formula (PBM-6-2), or Formula (PBM-6-3):

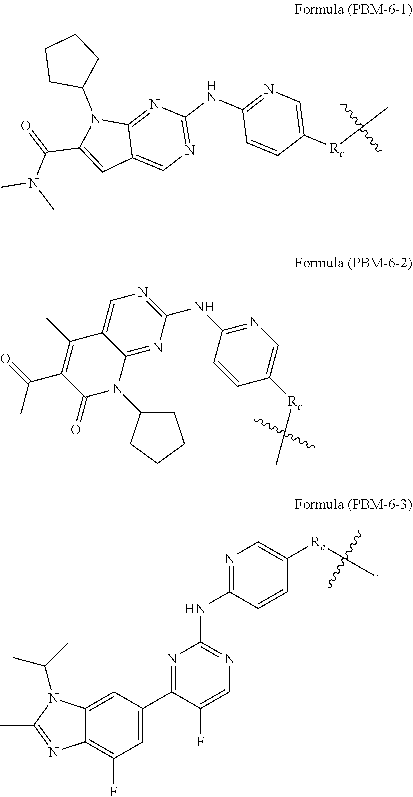

In some embodiments, PBM represents a small molecule ligand of ALK.

In some embodiments, PBM represents the structure of Formula (PBM-7):

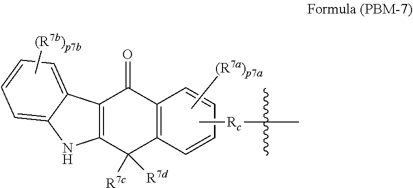

wherein (R^{7a})ₚ₇ₐ in Formula (PBM-7) indicates that the benzene ring to which it is attached is optionally substituted with p7a R^{7a}, wherein each R^{7a} independently represents deuterium, cyano, halogen (e.g., fluorine, chlorine, bromine, or iodine), C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), C₁₋₆ alkoxy (e.g., C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), or deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), and p7a represents an integer of 0, 1, 2, or 3; and
(R^{7b})_{p7b} indicates that the benzene ring to which it is attached is optionally substituted with p7b R^{7b}, wherein each R^{7b} independently represents deuterium, cyano, halogen (e.g., fluorine, chlorine, bromine, or iodine), C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), C₁₋₆ alkoxy (e.g., C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), or deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), and p7b represents an integer of 0, 1, 2, 3, or 4, and
R^{7c} and R^{7d} are the same or different and each independently represent deuterium, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), halogen (e.g., fluorine, chlorine, bromine, or iodine), halogenated C₁₋₆ alkyl (e.g., fluorine, chlorine, bromine, or iodine), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), C₁₋₆ alkoxy (e.g., C₁₋₄ alkoxy, such as methoxy, ethoxy, propoxy, isopropoxy, n-butoxy, sec-butoxy, or tert-butoxy), or halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—).

In some embodiments, PBM represents the structure of Formula (PBM-7-1):

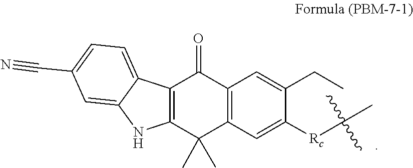

In some embodiments, PBM represents the structure of Formula (PBM-8):

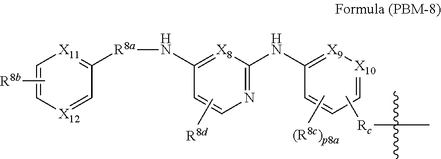

wherein X₈, X₉, X₁₀, X₁₁, and X₁₂ in Formula (PBM-8) are the same or different and independently represent N or CH;
R^{8a} represents a bond or optionally substituted methylene (e.g., halogenated methylene);
R^{8b} represents —C(O)N(R^{8f}) R^{8e}, —N(R^{8f})C(O)R^{8e}, —S(O)₂N(R^{8f})R^{8e}, —N(R^{8f})S(O)₂R^{8e}, —S(O)₂R^{8e}, or —P(O)(R^{8e})₂, wherein each R^{8e} is the same or different and independently represents C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), or deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), and R^{8f} represents hydrogen, or C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl);
(R^{8c})ₚ₈ₐ in Formula (PBM-8) indicates that the 6-membered ring containing X₉ and X₁₀ to which it is attached is optionally substituted with p8a R^{8c}, with each R^{8c} being the same or different and independently representing deuterium, halogen (e.g., fluorine, chlorine, bromine, or iodine), hydroxy, amino, cyano, C₁₋₆ alkoxy (e.g., C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), or halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—);
p8a represents an integer of 0, 1, 2, 3, or 4, and
R^{8d} represents deuterium, halogen (e.g., fluorine, chlorine, bromine, or iodine), hydroxy, amino, cyano, C₁₋₆ alkoxy (e.g., C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), or halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—).

In some embodiments, R^{8a} in Formula (PBM-8) represents a bond. In other words, the 6-membered ring containing X₁₁ and X₁₂ in Formula (PBM-8) is directly bonded to the —NH— group.

In some embodiments, R^{8a} in Formula (PBM-8) represents optionally substituted methylene (e.g., halogenated methylene).

In some embodiments, PBM represents the structure of Formula (PBM-8-1A):

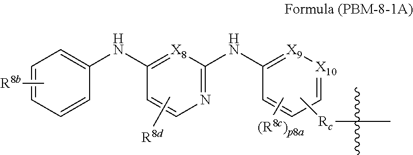

wherein X₈, X₉, and X₁₀ are the same or different and independently represent N or CH;
R^{8b} represents-C(O)N(R^{8f})R^{8e}, —N(R^{8f})C(O)R^{8e}, —S(O)₂N(R^{8f})R^{8e}, —N(R^{8f})S(O)₂R^{8e}, —S(O)₂R^{8e}, or —P(O)(R^{8e})₂, wherein each R^{8e} is the same or different and independently represents C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), or deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), and R^{8f} represents hydrogen or C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl);
(R^{8c})ₚ₈ₐ indicates that the 6-membered ring containing X₉ and X₁₀ to which it is attached is optionally substituted with p8a R^{8c}, with each R^{8c} being the same or different and independently representing deuterium, halogen (e.g., fluorine, chlorine, bromine, or iodine), hydroxy, amino, cyano, C₁₋₆ alkoxy (e.g., C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), or halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—);
p8a represents an integer of 0, 1, 2, 3, or 4; and
R^{8d} represents deuterium, halogen (e.g., fluorine, chlorine, bromine, or iodine), hydroxy, amino, cyano, C₁₋₆ alkoxy (e.g., C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), or halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—).

In some embodiments, PBM represents the structure of Formula (PBM-8-1B):

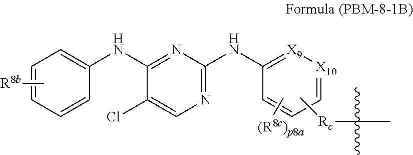

wherein X₉ and X₁₀ are the same or different and independently represent N or CH;
R^{8b} represents —C(O)N(R^{8f})R^{8e}, —N(R^{8f})C(O)R^{8e}, —S(O)₂N(R^{8f}) R^{8e}, —N(R^{8f})S(O)₂R^{8e}, —S(O)₂R^{8e}, or —P(O)(R^{8e})₂, wherein each R^{8e} is the same or different and independently represents C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), or deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), and R^{8f} represents hydrogen, or C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl);
(R^{8c})ₚ₈ₐ indicates that the 6-membered ring containing X₉ and X₁₀ to which it is attached is optionally substituted with p8a R^{8c}, with each R^{8c} being the same or different and independently representing deuterium, halogen (e.g., fluorine, chlorine, bromine, or iodine), hydroxy, amino, cyano, C₁₋₆ alkoxy (e.g., C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), or halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—); and
p8a represents an integer of 0, 1, 2, 3, or 4.

In some embodiments, PBM represents the structure of Formula (PBM-8-1) or Formula (PBM-8-2):

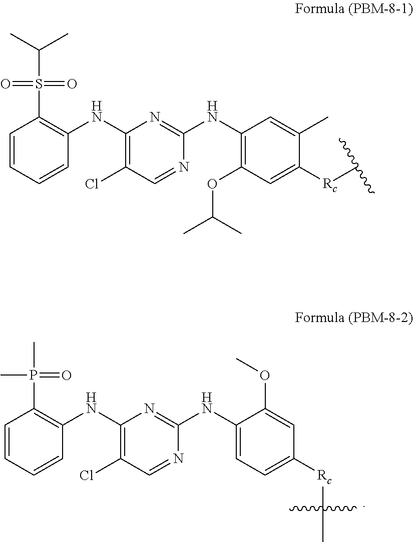

In some embodiments, PBM represents a small molecule ligand of BCR-ABL.

In some embodiments, PBM represents the structure of Formula (PBM-9):

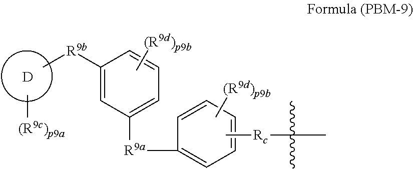

wherein R^{9a} represents —NHC(O)— or —C(O)NH—;
R^{9b} represents —NH—, —N(C₁₋₆ alkyl)-, or ethynylene;
ring D represents a 5-to 20-membered monocyclic, bicyclic, or polycyclic heteroaryl group containing 1 to 4 heteroatoms selected from N, O, and S, and (R^{9c})ₚ₉ₐ indicates that the ring D is optionally substituted with p9a R^{9c}, with each R^{9c} being the same or different and each independently representing optionally substituted 5-to 15-membered heteroaryl, deuterium, halogen (e.g., fluorine, chlorine, bromine, or iodine), hydroxy, cyano, amino, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), C₁₋₆ alkoxy (e.g., C₁₋₄ alkoxy, such as methoxy, ethoxy, propoxy, isopropoxy, n-butoxy, sec-butoxy, or tert-butoxy), or halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—);
p9a represents an integer of 0, 1, 2, or 3;
each (R^{9d})_{p9b} in Formula (PBM-9) independently indicates that the benzene ring to which it is attached is independently optionally substituted with p9b R^{9d}, with each Rod being the same or different and each independently representing deuterium, halogen (e.g., fluorine, chlorine, bromine, or iodine), hydroxy, cyano, amino, C₁₋₆ alkoxy (e.g., C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), or halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), and
each p9b is the same or different and independently represents an integer of 0, 1, 2, 3, or 4.

In some embodiments, ring D in Formula (PBM-9) represents a 5-to 20-membered monocyclic, bicyclic, or polycyclic heteroaryl group containing 1 to 4 heteroatoms selected from N, O, and S. In a sub-embodiment, examples of 5-to 20-membered monocyclic, bicyclic, or polycyclic heteroaryl include, but are not limited to, furanyl, oxazolyl, isoxazolyl, oxadiazolyl, thienyl, thiazolyl, isothiazolyl, thiadiazolyl, pyrrolyl, imidazolyl, pyrazolyl, triazolyl, pyridyl, pyrimidinyl, pyridazinyl, pyrazinyl, indolyl, isoindolyl, benzofuranyl, isobenzofuranyl, benzothienyl, indazolyl, benzimidazolyl, benzoxazolyl, benzisoxazolyl, benzothiazolyl, benzisothiazolyl, benzotriazolyl, benzo[2,1,3]oxadiazolyl, benzo[2,1,3]thiadiazolyl, benzo[1,2,3]thiadiazolyl, quinolinyl, isoquinolinyl, naphthyridinyl, cinnolinyl, quinazolinyl, quinoxalinyl, phthalazinyl, pyrazolo[1,5-alpyridyl, pyrazolo[1,5-a]pyrimidinyl, imidazo[1,2-a]pyridyl, 1H-pyrrolo[3,2-b]pyridyl, 1H-pyrrolo[2,3-b]pyridyl, pyrrolo[2,1-b]thiazolyl, imidazo[2,1-b]thiazolyl, and imidazo[1,2-b]pyridazinyl. The heteroaryl group is optionally substituted with p9a R^{9c}, wherein p9a represents an integer of 0, 1, 2, or 3, and wherein each R^{9c} is the same or different and independently represents: optionally substituted 5-to 15-membered heteroaryl, deuterium, halogen (e.g., fluorine, chlorine, bromine, or iodine), hydroxy, cyano, amino, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), C₁₋₆ alkoxy (e.g., C₁₋₄ alkoxy, such as methoxy, ethoxy, propoxy, isopropoxy, n-butoxy, sec-butoxy, or tert-butoxy), or halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—).

In some embodiments, R^{9c} in Formula (PBM-9) represents optionally substituted 5-to 15-membered heteroaryl. In some embodiments, the 5-to 15-membered heteroaryl is 5-to 14-membered monocyclic or bicyclic aromatic ring group containing one or more (e.g., from 1 to 6, or from 1 to 5, or from 1 to 4, or from 1 to 3) heteroatoms selected from the group consisting of oxygen, nitrogen, and sulfur. In some sub-embodiments, examples of heteroaryl include, but are not limited to, furanyl, oxazolyl, isoxazolyl, oxadiazolyl, thienyl, thiazolyl, isothiazolyl, thiadiazolyl, pyrrolyl, imidazolyl, pyrazolyl, triazolyl, pyridyl, pyrimidinyl, pyridazinyl, pyrazinyl, indolyl, isoindolyl, benzofuranyl, isobenzofuranyl, benzothienyl, indazolyl, benzimidazolyl, benzoxazolyl, benzisoxazolyl, benzothiazolyl, benzisothiazolyl, benzotriazolyl, benzo[2,1,3]oxadiazolyl, benzo[2,1,3]thiadiazolyl, benzo[1,2,3]thiadiazolyl, quinolinyl, isoquinolinyl, naphthyridinyl, cinnolinyl, quinazolinyl, quinoxalinyl, phthalazinyl, pyrazolo[1,5-a]pyridyl, pyrazolo[1,5-alpyrimidinyl, imidazo[1,2-a]pyridyl, 1H-pyrrolo[3,2-b]pyridyl, 1H-pyrrolo[2,3-b]pyridyl, pyrrolo[2,1-b]thiazolyl, imidazo[2,1-b]thiazolyl, and imidazo[1,2-b]pyridazinyl. The heteroaryl group is optionally substituted with one or more (e.g., 1-6, such as 1, 2, 3, 4, 5, or 6) substituents optionally selected from the group consisting of: halogen (e.g., fluorine, chlorine, bromine, or iodine), hydroxy, cyano, amino, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), C₁₋₆ alkoxy (e.g., C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), and any combination thereof.

In some embodiments, Rob in Formula (PBM-9) represents-NH—.

In some embodiments, Rob in Formula (PBM-9) represents ethynylene.

In some embodiments, PBM represents the structure of Formula (PBM-9-1A):

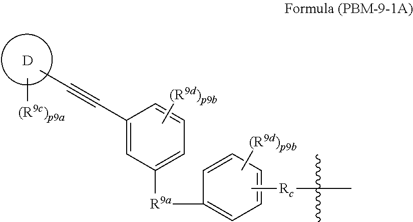

wherein R^{9a} represents —NHC(O)— or —C(O)NH—;
ring D represents a 5-to 20-membered monocyclic, bicyclic, or polycyclic heteroaryl group containing 1 to 4 heteroatoms selected from N, O, and S, and (R^{9c})ₚ₉ₐ indicates that the ring D is optionally substituted with p9a R^{9c}, with each Roc being the same or different and each independently representing optionally substituted 5-to 15-membered heteroaryl, deuterium, halogen (e.g., fluorine, chlorine, bromine, or iodine), hydroxy, cyano, amino, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), C₁₋₆ alkoxy (e.g., C₁₋₄ alkoxy, such as methoxy, ethoxy, propoxy, isopropoxy, n-butoxy, sec-butoxy, or tert-butoxy), or halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—);
p9a represents an integer of 0, 1, 2, or 3;
each (R^{9d})_{p9b} in Formula (PBM-9-1A) independently indicates that the benzene ring to which it is attached is independently optionally substituted with p9b R^{9d}, with each Rod being the same or different and each independently representing deuterium, halogen (e.g., fluorine, chlorine, bromine, or iodine), hydroxy, cyano, amino, C₁₋₆ alkoxy (e.g., C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), or halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), and
each p9b is the same or different and independently represents an integer of 0, 1, 2, 3, or 4.

In some embodiments, ring D in Formula (PBM-9-1A) represents a 5-to 20-membered monocyclic, bicyclic, or polycyclic heteroaryl group containing 1 to 4 heteroatoms selected from N, O, and S. In a sub-embodiment, examples of 5-to 20-membered monocyclic, bicyclic, or polycyclic heteroaryl include, but are not limited to, furanyl, oxazolyl, isoxazolyl, oxadiazolyl, thienyl, thiazolyl, isothiazolyl, thiadiazolyl, pyrrolyl, imidazolyl, pyrazolyl, triazolyl, pyridyl, pyrimidinyl, pyridazinyl, pyrazinyl, indolyl, isoindolyl, benzofuranyl, isobenzofuranyl, benzothienyl, indazolyl, benzimidazolyl, benzoxazolyl, benzisoxazolyl, benzothiazolyl, benzisothiazolyl, benzotriazolyl, benzo[2,1,3]oxadiazolyl, benzo[2,1,3]thiadiazolyl, benzo[1,2,3]thiadiazolyl, quinolinyl, isoquinolinyl, naphthyridinyl, cinnolinyl, quinazolinyl, quinoxalinyl, phthalazinyl, pyrazolo[1,5-alpyridyl, pyrazolo[1,5-a]pyrimidinyl, imidazo[1,2-a]pyridyl, 1H-pyrrolo[3,2-b]pyridyl, 1H-pyrrolo[2,3-b]pyridyl, pyrrolo[2,1-b]thiazolyl, imidazo[2,1-b]thiazolyl, and imidazo[1,2-b]pyridazinyl. The heteroaryl group is optionally substituted with p9a R^{9c}, wherein p9a represents an integer of 0, 1, 2, or 3, and each Roc is the same or different and independently represents: optionally substituted 5-to 15-membered heteroaryl, deuterium, halogen (e.g., fluorine, chlorine, bromine, or iodine), hydroxy, cyano, amino, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), C₁₋₆ alkoxy (e.g., C₁₋₄ alkoxy, such as methoxy, ethoxy, propoxy, isopropoxy, n-butoxy, sec-butoxy, or tert-butoxy), or halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—).

In some embodiments, Roc in Formula (PBM-9-1A) represents optionally substituted 5-to 15-membered heteroaryl. In some embodiments, the 5-to 15-membered heteroaryl is 5-to 14-membered monocyclic, bicyclic, or polycyclic aromatic ring group containing one or more (e.g., from 1 to 6, or from 1 to 5, or from 1 to 4, or from 1 to 3) heteroatoms selected from the group consisting of oxygen, nitrogen, and sulfur. In some sub-embodiments, examples of heteroaryl include, but are not limited to, furanyl, oxazolyl, isoxazolyl, oxadiazolyl, thienyl, thiazolyl, isothiazolyl, thiadiazolyl, pyrrolyl, imidazolyl, pyrazolyl, triazolyl, pyridyl, pyrimidinyl, pyridazinyl, pyrazinyl, indolyl, isoindolyl, benzofuranyl, isobenzofuranyl, benzothienyl, indazolyl, benzimidazolyl, benzoxazolyl, benzisoxazolyl, benzothiazolyl, benzisothiazolyl, benzotriazolyl, benzo[2,1,3]oxadiazolyl, benzo[2,1,3]thiadiazolyl, benzo[1,2,3]thiadiazolyl, quinolinyl, isoquinolinyl, naphthyridinyl, cinnolinyl, quinazolinyl, quinoxalinyl, phthalazinyl, pyrazolo[1,5-a]pyridyl, pyrazolo[1,5-alpyrimidinyl, imidazo[1,2-a]pyridyl, 1H-pyrrolo[3,2-b]pyridyl, 1H-pyrrolo[2,3-b]pyridyl, pyrrolo[2,1-b]thiazolyl, imidazo[2,1-b]thiazolyl, and imidazo[1,2-b]pyridazinyl. The heteroaryl group is optionally substituted with one or more (e.g., 1-6, such as 1, 2, 3, 4, 5, or 6) substituents optionally selected from the group consisting of: halogen (e.g., fluorine, chlorine, bromine, or iodine), hydroxy, cyano, amino, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), C₁₋₆ alkoxy (e.g., C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), and any combination thereof.

In some embodiments, PBM represents the structure of Formula (PBM-9-1):

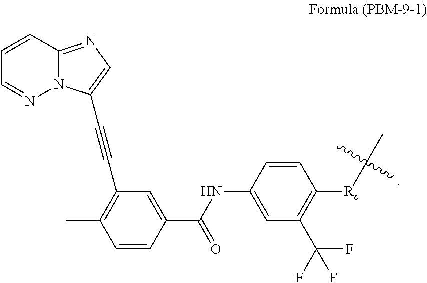

In some embodiments, PBM represents the structure of Formula (PBM-21):

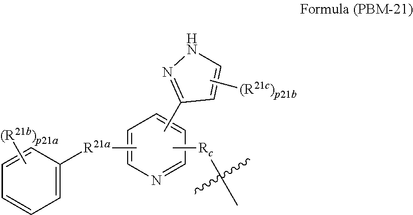

wherein R^{21a} in Formula (PBM-21) represents NHC(O), C(O)NH, NHS(O)₂, or S(O)₂NH;
(R^{21b})ₚ₂₁ₐ indicates that the benzene ring to which it is attached is optionally substituted with p21a R^{21b} wherein each R^{21b} is independently halogen (e.g., fluorine, chlorine, bromine, or iodine), hydroxy, cyano, amino, nitro, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), C₁₋₆ alkoxy (e.g., C₁₋₄ alkoxy, such as methoxy, ethoxy, propoxy, isopropoxy, n-butoxy, sec-butoxy, or tert-butoxy), or halogenated C₁₋₆ alkoxy (e.g., halogenated C₁₋₄ alkoxy, such as F₃C—O—, FCH₂—O—, F₂CH—O—, F₂ClC—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), and p21a represents an integer of 0, 1, 2, 3, 4, or 5, and
(R^{21c})_{p21b} indicates that the 1H-pyrazole ring to which it is attached is optionally substituted with p21b R^{21c}, wherein each R^{21c} is independently halogen (e.g., fluorine, chlorine, bromine, or iodine), hydroxy, cyano, amino, nitro, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), C₁₋₆ alkoxy (e.g., C₁₋₄ alkoxy, such as methoxy, ethoxy, propoxy, isopropoxy, n-butoxy, sec-butoxy, or tert-butoxy), or halogenated C₁₋₆ alkoxy (e.g., halogenated C₁₋₄ alkoxy, such as F₃C—O—, FCH₂—O—, F₂CH—O—, F₂ClCH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), and p21b represents an integer of 0, 1 or 2.

In some embodiments, p21a in Formula (PBM-21) represents an integer of 1, and R^{21b} is halogen (e.g., fluorine, chlorine, bromine, or iodine), hydroxy, cyano, amino, nitro, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), C₁₋₆ alkoxy (e.g., C₁₋₄ alkoxy, such as methoxy, ethoxy, propoxy, isopropoxy, n-butoxy, sec-butoxy, or tert-butoxy), or halogenated C₁₋₆ alkoxy (e.g., halogenated C₁₋₄ alkoxy, such as F₃C—O—, FCH₂—O—, F₂CH—O—, F₂ClCH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—). In some embodiments, R^{21b} is F₂ClC—O—.

In some embodiments, R^{21a} represents NHC(O). In some embodiments, R^{21a} represents C(O)NH.

In some embodiments, p21b represents an integer of 0.

In some embodiments, PBM represents the structure of Formula (PBM-21-1):

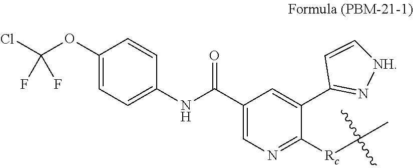

In some embodiments, PBM represents a small molecule ligand of FAK.

In some embodiments, PBM represents the structure of Formula (PBM-8-1C):

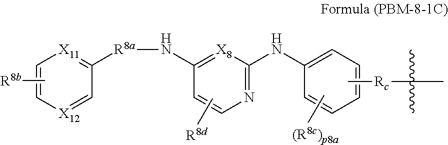

wherein X₈, X₁₁, and X₁₂ in Formula (PBM-8-1C) are the same or different and independently represent N or CH;
R^{8a} represents a bond or optionally substituted methylene (e.g., halogenated methylene);
R^{8b} represents —C(O)N(R^{8f}) R^{8e}, —N(R^{8f})C(O)R^{8a}, —S(O)₂N(R^{8f})R^{8c}, —N(R^{8f})S(O)₂R^{8e}, —S(O)₂R^{8e}, or —P(O)(R^{8e})₂, wherein each R^{8e} is the same or different and independently represents C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), or deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), and R^{8f} represents hydrogen or C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl);
(R^{8c})ₚ₈ₐ in Formula (PBM-8-1C) indicates that the benzene ring to which it is attached is optionally substituted with p8a R^{8c}, wherein each R^{8c} is the same or different and independently represents deuterium, halogen (e.g., fluorine, chlorine, bromine, or iodine), hydroxy, amino, cyano, C₁₋₆ alkoxy (e.g., C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, F₂ClCH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), or halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—);
p8a represents an integer of 0, 1, 2, 3, or 4, and
R^{8d} represents deuterium, halogen (e.g., fluorine, chlorine, bromine, or iodine), hydroxy, amino, cyano, C₁₋₆ alkoxy (e.g., C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, F₂ClCH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), or halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—).

In some embodiments, R^{8a} in Formula (PBM-8-1C) represents a bond. In other words, the 6-membered ring containing X₁₁ and X₁₂ in Formula (PBM-8-1C) is directly bonded to the—NH— group.

In some embodiments, R^{8a} in Formula (PBM-8-1C) represents optionally substituted methylene (e.g., halogenated methylene).

In some embodiments, PBM represents the structure of Formula (PBM-8-1D) or Formula (PBM-8-1E):

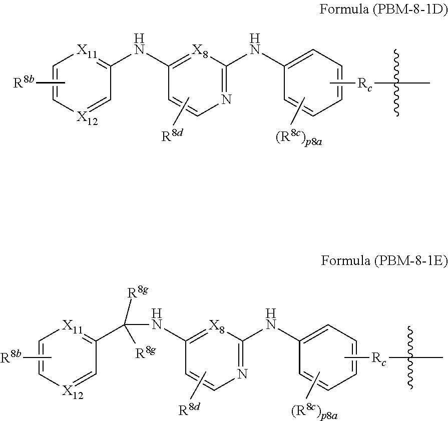

wherein X₈, X₁₁, and X₁₂ are the same or different and independently represent N or CH;
each R^{8g} is the same or different and independently represents hydrogen or halogen;
R^{8b} represents-C(O)N(R^{8f}) R^{8e}, —N(R^{8f})C(O)R^{8e}, —S(O)₂N(R^{8f}) R^{8e}, —N(R^{8f})S(O)₂R^{8e}, —S(O)₂R^{8e}, or —P(O)(R^{8e})₂, wherein each R^{8e} is the same or different and independently represents C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), or deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), and R^{8f} represents hydrogen, or C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl);
(R^{8c})ₚ₈ₐ indicates that the benzene ring to which it is attached is optionally substituted with p8a R^{8c}, wherein each R^{8c} is the same or different and independently represents deuterium, halogen (e.g., fluorine, chlorine, bromine, or iodine), hydroxy, amino, cyano, C₁₋₆ alkoxy (e.g., C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, F₂ClCH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), or halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—);
p8a represents an integer of 0, 1, 2, 3, or 4, and
R^{8d} represents deuterium, halogen (e.g., fluorine, chlorine, bromine, or iodine), hydroxy, amino, cyano, C₁₋₆ alkoxy (e.g., C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, F₂ClCH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), or halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—).

In some embodiments, PBM represents the structure of Formula (PBM-8-3), Formula (PBM-8-4), Formula (PBM-8-5), Formula (PBM-8-6), Formula (PBM-8-7), or Formula (PBM-8-8):

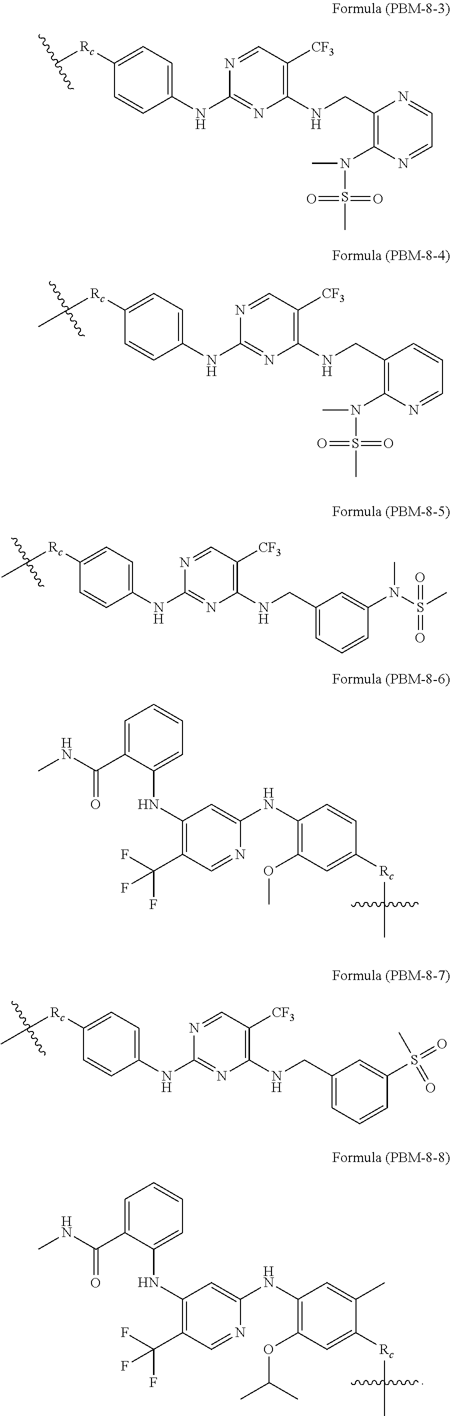

In some embodiments, PBM represents the structure of Formula (PBM-22):

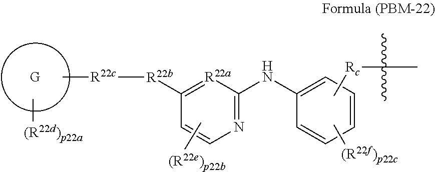

wherein R^{22a} represents N or CH;
R^{22b} represents O, S, NH, CH₂, CH(F), or C(F)₂;
R^{22c} represents a bond, or optionally substituted methylene (e.g., halogenated methylene);
ring G represents C₆₋₁₅ aryl, 4-to 15-membered heterocyclyl, or 5-to 15-membered heteroaryl, and (R^{22d})ₚ₂₂ₐ indicates that the ring G to which it is attached is optionally substituted with p22a R^{22d}, wherein each R^{22d} is the same or different and each independently represents deuterium, halogen (e.g., fluorine, chlorine, bromine, or iodine), hydroxy, cyano, amino, nitro, oxo, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), C₁₋₆ alkoxy (e.g., C₁₋₄ alkoxy, such as methoxy, ethoxy, propoxy, isopropoxy, n-butoxy, sec-butoxy, or tert-butoxy), or halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), and p22a represents an integer of 0, 1, 2, 3, or 4;
(R^{22e})_{p22b} indicates that the 6-membered nitrogen-containing heteroaromatic ring containing R^{22a} to which it is attached is optionally substituted with p22b R^{22e}, and each R^{22e} represents deuterium, halogen (e.g., fluorine, chlorine, bromine, or iodine), hydroxy, cyano, amino, nitro, C₁₋₆ alkoxy (e.g., C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, F₂ClCH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), or halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), and p22b represents an integer of 0, 1 or 2;
(R^{22f})_{p22c} indicates that the benzene ring to which it is attached is optionally substituted with p22c R^{22f}, wherein each R^{22f} is the same or different and independently represents deuterium, halogen (e.g., fluorine, chlorine, bromine, or iodine), hydroxy, cyano, amino, nitro, C₁₋₆ alkoxy (e.g., C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, F₂ClCH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), or halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), and p22c represents an integer of 0, 1, 2, 3, or 4.

In some embodiments, R^{22a} represents N.

In some embodiments, R^{22a} represents CH.

In some embodiments, R^{22c} represents a bond.

In some embodiments, R^{22c} represents optionally substituted methylene (e.g., methylene optionally substituted with halogen (e.g., fluorine, chlorine, bromine, or iodine)).

In some embodiments, ring G represents C₆₋₁₅ aryl, 4-to 15-membered heterocyclyl, or 5-to 15-membered heteroaryl. Examples of C₆₋₁₅ aryl include, but are not limited to, phenyl and naphthyl. Examples of 4-to 15-membered heterocyclyl include, but are not limited to, azetidinyl, oxetanyl, pyrrolidinyl, imidazolidinyl, pyrazolidyl, tetrahydrofuranyl, tetrahydropyranyl, tetrahydrothienyl, tetrahydrothiopyranyl, oxazolidinyl, thiazolidinyl, piperidinyl, piperazinyl, morpholinyl, thiomorpholinyl, dioxacyclohexyl, azacycloheptyl, diazacycloheptanyl (e.g., 1,4-diazacycloheptan-1-yl), azacyclooctyl, diazacyclooctyl, azabicyclo[3.1.1]heptanyl, azabicyclo[2.2.1]heptanyl, azabicyclo[3.2.1]octanyl, azabicyclo[2.2.2]octanyl, diazabicyclo[3.1.1]heptanyl, diazabicyclo[2.2.1]heptanyl, diazabicyclo[3.2.1]octanyl (e.g., 3,8-diazabicyclo[3.2.1]octan-3-yl), diazabicyclo[2.2.2]octanyl (e.g., 2,5-diazabicyclo[2.2.2]octan-2-yl). Examples of 5-to 15-membered heteroaryl include, but are not limited to, furanyl, oxazolyl, isoxazolyl, oxadiazolyl, thienyl, thiazolyl, isothiazolyl, thiadiazolyl, pyrrolyl, imidazolyl, pyrazolyl, triazolyl, pyridyl, pyrimidinyl, pyridazinyl, pyrazinyl, indolyl, isoindolyl, isoindolinyl, benzofuranyl, isobenzofuranyl, benzothienyl, indazolyl, benzimidazolyl, benzoxazolyl, benzisoxazolyl, benzothiazolyl, benzisothiazolyl, benzotriazolyl, benzo[2,1,3]oxadiazolyl, benzo[2,1,3]thiadiazolyl, benzo[1,2,3]thiadiazolyl, quinolinyl, isoquinolinyl, naphthyridinyl, cinnolinyl, quinazolinyl, quinoxalinyl, phthalazinyl, pyrazolo[1,5-a]pyridyl, pyrazolo[1,5-alpyrimidinyl, imidazo[1,2-a]pyridyl, 1H-pyrrolo[3,2-b]pyridyl, 1H-pyrrolo[2,3-b]pyridyl, 4H-furo[3,2-b]pyrrolyl, pyrrolo[2,1-b]thiazolyl, and imidazo[2,1-b]thiazolyl.

In some embodiments, (R^{22d})ₚ₂₂ₐ in Formula (PBM-22) indicates that the ring G to which it is attached is optionally substituted with p22a R^{22d}, wherein each R^{22d} is the same or different and each independently represents deuterium, halogen (e.g., fluorine, chlorine, bromine, or iodine), hydroxy, cyano, amino, nitro, oxo, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), C₁₋₆ alkoxy (e.g., C₁₋₄ alkoxy, such as methoxy, ethoxy, propoxy, isopropoxy, n-butoxy, sec-butoxy, or tert-butoxy), or halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), and p22a represents an integer of 0, 1, 2, 3, or 4.

In some embodiments, ring G represents isoindolinyl, which is optionally substituted with p22a R^{22d} and each R^{22d} is the same or different and each independently represents deuterium, halogen (e.g., fluorine, chlorine, bromine, or iodine), hydroxy, cyano, amino, nitro, oxo, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), C₁₋₆ alkoxy (e.g., C₁₋₄ alkoxy, such as methoxy, ethoxy, propoxy, isopropoxy, n-butoxy, sec-butoxy, or tert-butoxy), or halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), and p22a represents an integer of 0, 1, 2, 3, or 4. In some embodiments, p22a represents an integer of 0, 1, 2, 3, or 4. In some embodiments, ring G represents isoindolinyl, which is optionally substituted with halogen (e.g., fluorine, chlorine, bromine, or iodine), oxo, and C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl).

In some embodiments, (R^{22e})_{p22b} indicates that the 6-membered nitrogen-containing heteroaromatic ring containing R^{22a} to which it is attached is optionally substituted with p22b R^{22e}, and each R^{22e} represents deuterium, halogen (e.g., fluorine, chlorine, bromine, or iodine), hydroxy, cyano, amino, nitro, C₁₋₆ alkoxy (e.g., C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), or halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), and p22b represents an integer of 0, 1 or 2. In some embodiments, each R^{22e} independently represents deuterium, halogen (e.g., fluorine, chlorine, bromine, or iodine), hydroxy, cyano, amino, nitro, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), or halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), and p22b represents an integer of 0, 1 or 2.

In some embodiments, (R^{22f})_{p22c} indicates that the benzene ring to which it is attached is optionally substituted with p22c R^{22f}, wherein each R^{22f} is the same or different and independently represents deuterium, halogen (e.g., fluorine, chlorine, bromine, or iodine), hydroxy, cyano, amino, nitro, C₁₋₆ alkoxy (e.g., C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), or halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), and p22c represents an integer of 0, 1, 2, 3, or 4. In some embodiments, each R^{22f} independently represents halogen (e.g., fluorine, chlorine, bromine, or iodine), or C₁₋₆ alkoxy (e.g., C₁₋₄ alkoxy, such as methoxy, ethoxy, propoxy, isopropoxy, n-butoxy, sec-butoxy, or tert-butoxy), and p22c represents an integer of 0, 1, 2, 3, or 4.

In some embodiments, PBM represents the structure of Formula (PBM-22-1A), Formula (PBM-22-1B), Formula (PBM-22-1C), Formula (PBM-22-2A), Formula (PBM-22-2B), or Formula (PBM-22-2C):

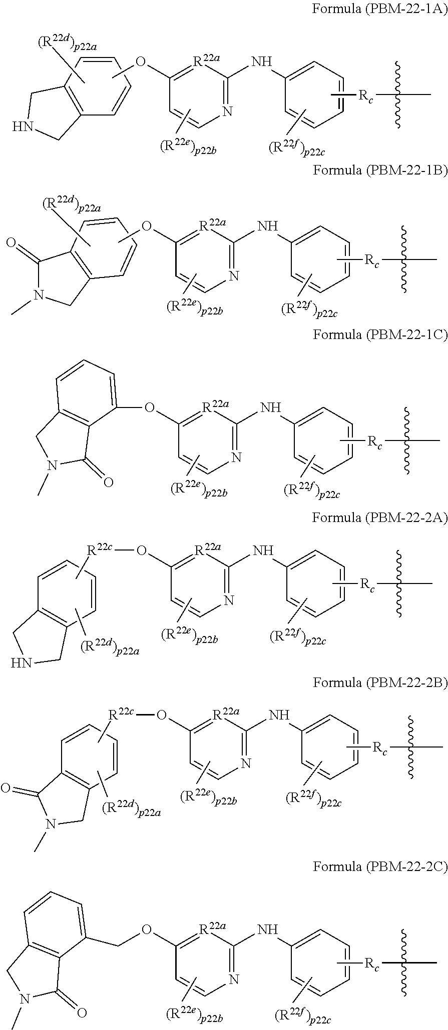

wherein R^{22a}, R^{22c}, (R^{22d})ₚ₂₂ₐ, (R^{22e})_{p22b}, and (R^{22f})_{p22c} are as defined in various embodiments of Formula (PBM-22) and each sub-embodiments thereof.

In some embodiments, PBM represents a structure of Formula (PBM-22-1), Formula (PBM-22-2), Formula (PBM-22-3), or Formula (PBM-22-4):

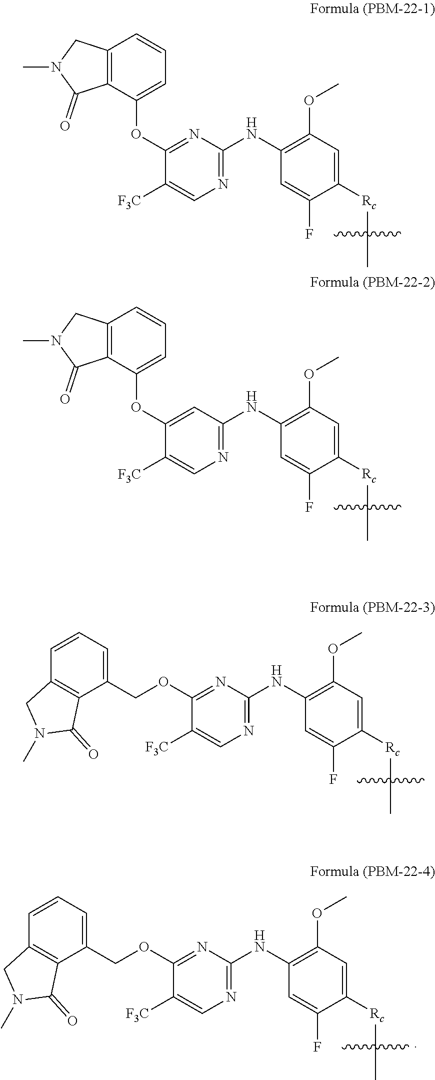

In some embodiments, PBM represents a small molecule ligand of ER.

In some embodiments, PBM represents a structure of Formula (PBM-10-1A), or Formula (PBM-10-1B) (which are non-limiting embodiments of the structure of Formula (PBM-10)):

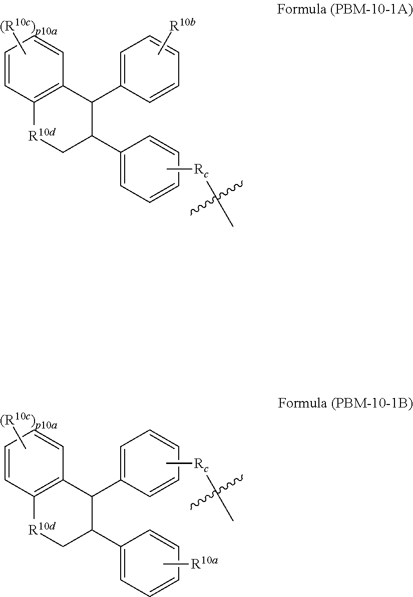

wherein each R^{10d} independently represents O, S, or CH₂;
(R^{10c})ₚ₁₀ₐ indicates that the benzo-fused six-membered ring containing R^{10d} is optionally substituted with p10a R^{10c}, with each R^{10c} being the same or different and independently representing hydrogen, hydroxy, halogen (e.g., fluorine, chlorine, bromine, or iodine), hydroxy, cyano, amino, C₁₋₆ alkoxy (e.g., C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), or halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—);
p10a represents an integer of 0, 1, 2, 3, or 4; and
R^{10a} and R^{10b} each independently represent hydrogen, halogen (e.g., fluorine, chlorine, bromine, or iodine), hydroxy, cyano, amino, C₁₋₆ alkoxy (e.g., C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), or halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—).

In some embodiments, PBM represents a structure of Formula (PBM-10-1), Formula (PBM-10-2), Formula (PBM-10-3), Formula (PBM-10-4), Formula (PBM-10-5), Formula (PBM-10-6), Formula (PBM-10-7), Formula (PBM-10-8), Formula (PBM-10-9), Formula (PBM-10-10), Formula (PBM-10-11), or Formula (PBM-10-12):

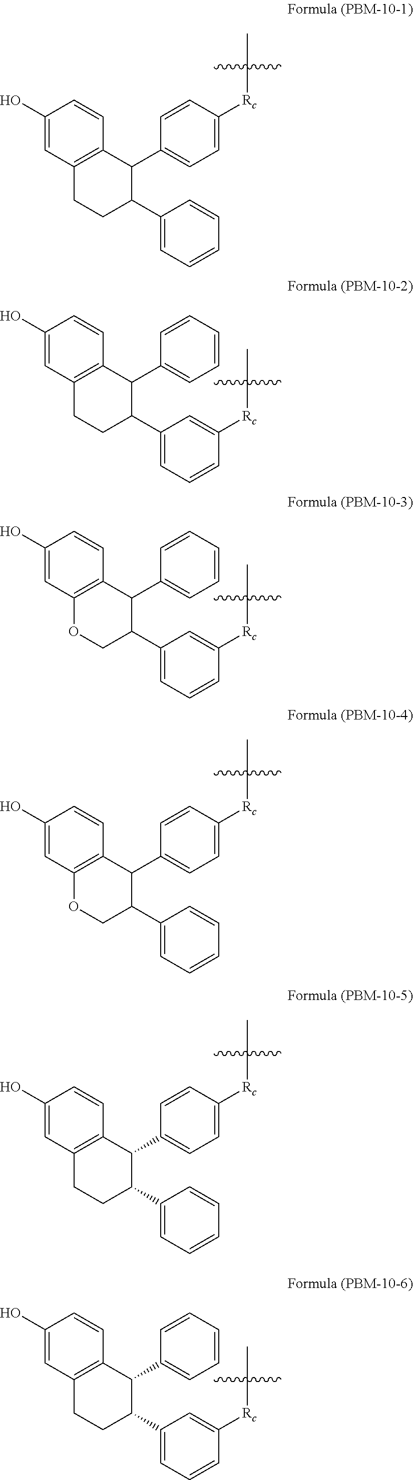

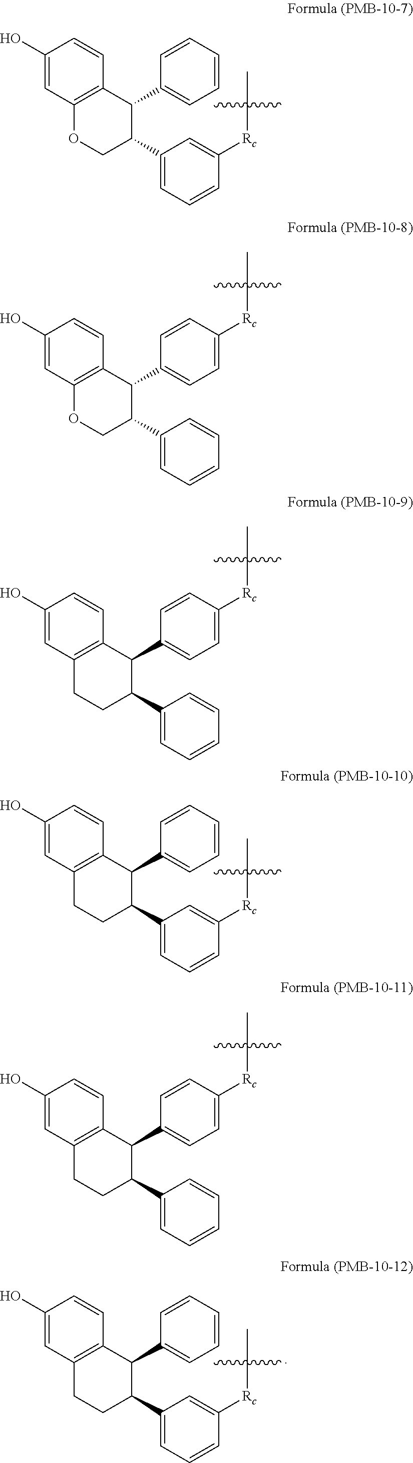

In some embodiments, PBM represents a small molecule ligand of IRAK4.

In some embodiments, PBM represents a structure of Formula (PBM-11):

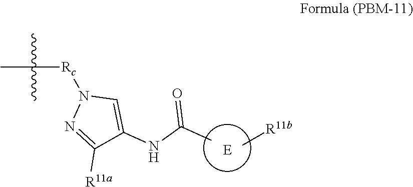

wherein ring E in Formula (PBM-11) represents a 5-to 20-membered monocyclic, bicyclic, or polycyclic heteroarylene group containing 1 to 4 heteroatoms selected from N, O, and S;
R^{11b} represents optionally substituted 5-to 15-membered heteroaryl, optionally substituted 5-to 15-membered heterocyclyl, deuterium, halogen (e.g., fluorine, chlorine, bromine, or iodine), hydroxy, cyano, amino, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), C₁₋₆ alkoxy (e.g., C₁₋₄ alkoxy, such as methoxy, ethoxy, propoxy, isopropoxy, n-butoxy, sec-butoxy, or tert-butoxy), or halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—);
R^{11a} represents hydrogen, halogen (e.g., fluorine, chlorine, bromine, or iodine), C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), C₁₋₆ alkoxy (e.g., C₁₋₄ alkoxy, such as methoxy, ethoxy, propoxy, isopropoxy, n-butoxy, sec-butoxy, or tert-butoxy), or halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—).

In some embodiments, ring E in Formula (PBM-11) represents a 5-to 20-membered monocyclic, bicyclic, or polycyclic heteroarylene group containing 1 to 4 heteroatoms selected from N, O, and S. In some embodiments, the 5-to 20-membered monocyclic, bicyclic, or polycyclic heteroarylene is 5-to 14-membered divalent monocyclic, bicyclic, or polycyclic aromatic ring group containing from 1 to 4 (e.g., from 1 to 4, or from 1 to 3, or from 1 to 2, or 1) heteroatoms selected from the group consisting of oxygen, nitrogen, and sulfur. In some sub-embodiments, examples of 5-to 20-membered monocyclic, bicyclic, or polycyclic heteroarylene include, but are not limited to, furanylene, oxazolylene, isoxazolylene, oxadiazolylene, thienylene, thiazolylene, isothiazolylene, thiadiazolylene, pyrrolylene, imidazolylene, pyrazolylene, triazolylene, pyridylene, pyrimidinylene, pyridazinylene, pyrazinylene, indolylene, isoindolylene, benzofuranylene, isobenzofuranylene, benzothienylene, indazolylene, benzimidazolylene, benzoxazolylene, benzisoxazolylene, benzothiazolylene, benzisothiazolylene, benzotriazolylene, benzo[2,1,3]oxadiazolylene, benzo[2,1,3]thiadiazolylene, benzo[1,2,3]thiadiazolylene, quinolinylene, isoquinolinylene, naphthyridinylene, cinnolinylene, quinazolinylene, quinoxalinylene, phthalazinylene, pyrazolo[1,5-a]pyridylene, pyrazolo[1,5-a]pyrimidinylene, imidazo[1,2-a]pyridylene, 1H-pyrrolo[3,2-b]pyridylene, 1H-pyrrolo[2,3-b]pyridylene, pyrrolo[2,1-b]thiazolylene, imidazo[2,1-b]thiazolylene, and imidazo[1,2-b]pyridazinylene. The 5-to 20-membered monocyclic, bicyclic, or polycyclic heteroarylene is optionally substituted with one or more (e.g., 1-3, such as 1, 2, or 3) R^{11b} substituents, wherein each R^{11b} independently represents optionally substituted 5-to 15-membered heteroaryl, optionally substituted 5-to 15-membered heterocyclyl, deuterium, halogen (e.g., fluorine, chlorine, bromine, or iodine), hydroxy, cyano, amino, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), C₁₋₆ alkoxy (e.g., C₁₋₄ alkoxy, such as methoxy, ethoxy, propoxy, isopropoxy, n-butoxy, sec-butoxy, or tert-butoxy), or halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—).

In some embodiments, R^{11b} in Formula (PBM-11) represents optionally substituted 5-to 15-membered heteroaryl. In some embodiments, 5-to 15-membered heteroaryl is 5-to 15-membered monocyclic, bicyclic, or polycyclic aromatic ring group containing from 1 to 4 (e.g., from 1 to 4, or from 1 to 3, or from 1 to 2, or 1) heteroatoms selected from the group consisting of oxygen, nitrogen, and sulfur. In some sub-embodiments, examples of heteroaryl include, but are not limited to, furanyl, oxazolyl, isoxazolyl, oxadiazolyl, thienyl, thiazolyl, isothiazolyl, thiadiazolyl, pyrrolyl, imidazolyl, pyrazolyl, triazolyl, pyridyl, pyrimidinyl, pyridazinyl, pyrazinyl, indolyl, isoindolyl, benzofuranyl, isobenzofuranyl, benzothienyl, indazolyl, benzimidazolyl, benzoxazolyl, benzisoxazolyl, benzothiazolyl, benzisothiazolyl, benzotriazolyl, benzo[2,1,3]oxadiazolyl, benzo[2,1,3]thiadiazolyl, benzo[1,2,3]thiadiazolyl, quinolinyl, isoquinolinyl, naphthyridinyl, cinnolinyl, quinazolinyl, quinoxalinyl, phthalazinyl, pyrazolo[1,5-a]pyridyl, pyrazolo[1,5-a]pyrimidinyl, imidazo[1,2-alpyridyl, 1H-pyrrolo[3,2-b]pyridyl, 1H-pyrrolo[2,3-b]pyridyl, pyrrolo[2,1-b]thiazolyl, imidazo[2,1-b]thiazolyl, and imidazo[1,2-b]pyridazinyl. Heteroaryl represented by R^{11b} is optionally substituted with one or more (e.g., 1-3, such as 1, 2, or 3) substituents which are optionally selected from the group consisting of:—N(R^{11c})—C₁₋₆ alkylene-optionally substituted C₃₋₈ cycloalkyl, —N(R^{11c})-optionally halogenated C₁₋₆ alkyl, halogen (e.g., fluorine, chlorine, bromine, or iodine), hydroxy, cyano, amino, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), C₁₋₆ alkoxy (e.g., C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), and any combination thereof, wherein each R^{11c} independently represents hydrogen or C₁₋₃ alkyl. In some embodiments, the substituent of heteroaryl represented by R^{11b} is —N(R^{11c})—C₁₋₆ alkylene-optionally substituted C₃₋₈ cycloalkyl, wherein R^{11c} represents hydrogen or C₁₋₃ alkyl (e.g., methyl). In some embodiments, examples of C₁₋₆ alkylene include, but are not limited to, C₁₋₃ alkylene. In some embodiments, examples of C₃₋₈ cycloalkyl include, but are not limited to, C₃₋₆ cycloalkyl. In some embodiments, representative examples of C₃₋₈ cycloalkyl include, but are not limited to, cyclopropyl, cyclobutyl, cyclopentyl, cyclopentenyl, cyclohexyl, cyclohexenyl, cycloheptyl, and cyclooctyl. C₃₋₈ cycloalkyl is optionally substituted with one or more (e.g., 1-3, such as 1, 2, or 3) substituents selected from the group consisting of C₁-C₃ alkyl, C₃-6 cycloalkyl, hydroxy, amino, mercapto, halogen, C₁-C₃ alkoxy, C₁-C₃ alkylamino, halogenated C₁-C₃ alkyl, amino-substituted C₁₋₃ alkylene, cyano, and any combination thereof. In some embodiments, the substituent of heteroaryl represented by R^{11b} is —N(R^{11c})-optionally halogenated C₁₋₆ alkyl, wherein R^{11c} represents hydrogen or C₁₋₃ alkyl (e.g., methyl). In some embodiments, examples of optionally halogenated C₁₋₆ alkyl include, but are not limited to, optionally halogenated C₁₋₄ alkyl, optionally halogenated C₁₋₃ alkyl, and optionally halogenated C₁₋₂ alkyl. In some embodiments, representative examples of optionally halogenated C₁₋₆ alkyl include, but are not limited to, —CH₂—CF₃.

In some embodiments, R^{11b} in Formula (PBM-11) represents optionally substituted 5-to 15-membered heterocyclyl. In some sub-embodiments, “5-to 15-membered heterocyclyl” refers to a 5-to 15-membered saturated or partially unsaturated (i.e., containing one or more double bonds, but not having a fully conjugated x-electron system) monocyclic, bicyclic, or tricyclic cyclic hydrocarbon group containing one or more (e.g., from 1 to 5, or from 1 to 4, from 1 to 3, from 1 to 2, or 1) heteroatoms independently selected from sulfur, oxygen, and nitrogen. In some sub-embodiments, examples of heterocyclyl include, but are not limited to, azetidinyl, oxetanyl, pyrrolidinyl, imidazolidinyl, pyrazolidyl, tetrahydrofuranyl, tetrahydropyranyl, tetrahydrothienyl, tetrahydrothiopyranyl, oxazolidinyl, thiazolidinyl, piperidinyl, piperazinyl, morpholinyl, thiomorpholinyl, dioxacyclohexyl, azacycloheptyl, azacyclooctyl, diazacycloheptanyl (e.g., 1,4-diazacycloheptan-1-yl), and diazacyclooctyl. The heterocyclyl is optionally substituted with one or more (e.g., 1 to 5, 1 to 4, 1 to 3, 1 to 2, or 1) substituents selected from the group consisting of deuterium, hydroxy, amino, mercapto, nitro, halogen (e.g., fluorine, chlorine, bromine, or iodine), cyano, C₁₋₆ alkyl, halogenated C₁₋₆ alkyl, deuterated C₁₋₆ alkyl, C₃-6 cycloalkyl, C₁₋₆ alkoxy, halogenated C₁₋₆ alkoxy, and any combination thereof.

In some embodiments, PBM represents the structure of Formula (PBM-11-1), Formula (PBM-11-2), Formula (PBM-11-3), or Formula (PBM-11-4):

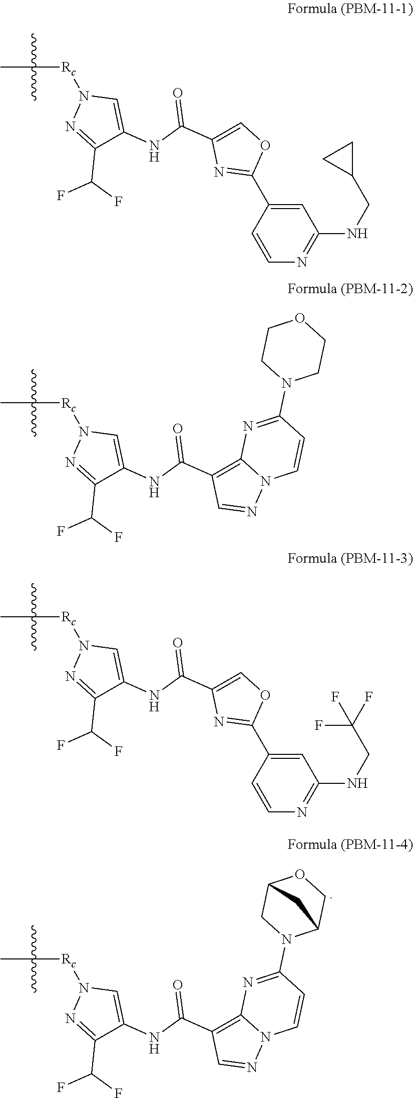

In some embodiments, PBM represents the structure of Formula (PBM-12):

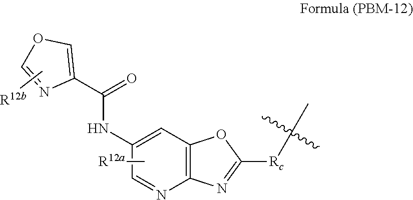

wherein R^{12a} in Formula (PBM-12) represents optionally substituted 5-to 15-membered heterocyclyl, optionally substituted 5-to 15-membered heteroaryl, deuterium, halogen (e.g., fluorine, chlorine, bromine, or iodine), hydroxy, cyano, amino, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), C₁₋₆ alkoxy (e.g., C₁₋₄ alkoxy, such as methoxy, ethoxy, propoxy, isopropoxy, n-butoxy, sec-butoxy, or tert-butoxy), or halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), and
R^{12b} represents a 5-to 20-membered monocyclic, bicyclic, or polycyclic heteroaryl group containing 1 to 4 heteroatoms selected from N, O, and S, and the heteroaryl is optionally substituted with one or more (e.g., 1-6, such as 1, 2, 3, 4, 5, or 6) substituents selected from the group consisting of deuterium, halogen (e.g., fluorine, chlorine, bromine, or iodine), hydroxy, cyano, amino, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), C₁₋₆ alkoxy (e.g., C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), and any combination thereof.

In some embodiments, R^{12a} in Formula (PBM-12) represents optionally substituted 5-to 15-membered heterocyclyl. In some sub-embodiments, “5-to 15-membered heterocyclyl” refers to a 5-to 15-membered saturated or partially unsaturated (i.e., containing one or more double bonds, but not having a fully conjugated x-electron system) monocyclic, bicyclic, or tricyclic cyclic hydrocarbon group containing one or more (e.g., from 1 to 5, or from 1 to 4, from 1 to 3, from 1 to 2, or 1) heteroatoms independently selected from sulfur, oxygen, and nitrogen. In some sub-embodiments, examples of heterocyclyl include, but are not limited to, azetidinyl, oxetanyl, pyrrolidinyl, imidazolidinyl, pyrazolidyl, tetrahydrofuranyl, tetrahydropyranyl, tetrahydrothienyl, tetrahydrothiopyranyl, oxazolidinyl, thiazolidinyl, piperidinyl, piperazinyl, morpholinyl, thiomorpholinyl, dioxacyclohexyl, azacycloheptyl, azacyclooctyl, diazacycloheptanyl (e.g., 1,4-diazacycloheptan-1-yl), and diazacyclooctyl. The heterocyclyl is optionally substituted with one or more (e.g., 1 to 5, 1 to 4, 1 to 3, 1 to 2, or 1) substituents selected from the group consisting of deuterium, hydroxy, amino, mercapto, nitro, halogen (e.g., fluorine, chlorine, bromine, or iodine), cyano, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), C₃₋₆ cycloalkyl, C₁₋₆ alkoxy (e.g., C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), and any combination thereof.

In some embodiments, R^{12b} in Formula (PBM-12) represents optionally substituted 5-to 20-membered (e.g., 5-to 15-membered, 5-to 10-membered, or 5-to 6-membered) monocyclic, bicyclic, or polycyclic heteroaryl containing from 1 to 4 heteroatoms selected from the group consisting of oxygen, nitrogen, and sulfur. In some sub-embodiments, examples of heteroaryl include, but are not limited to, furanyl, oxazolyl, isoxazolyl, oxadiazolyl, thienyl, thiazolyl, isothiazolyl, thiadiazolyl, pyrrolyl, imidazolyl, pyrazolyl, triazolyl, pyridyl, pyrimidinyl, pyridazinyl, pyrazinyl, indolyl, isoindolyl, benzofuranyl, isobenzofuranyl, benzothienyl, indazolyl, benzimidazolyl, benzoxazolyl, benzisoxazolyl, benzothiazolyl, benzisothiazolyl, benzotriazolyl, benzo[2,1,3]oxadiazolyl, benzo[2,1,3]thiadiazolyl, benzo[1,2,3]thiadiazolyl, quinolinyl, isoquinolinyl, naphthyridinyl, cinnolinyl, quinazolinyl, quinoxalinyl, phthalazinyl, pyrazolo[1,5-a]pyridyl, pyrazolo[1,5-alpyrimidinyl, imidazo[1,2-a]pyridyl, 1H-pyrrolo[3,2-b]pyridyl, 1H-pyrrolo[2,3-b]pyridyl, pyrrolo[2,1-b]thiazolyl, imidazo[2,1-b]thiazolyl, and imidazo[1,2-b]pyridazinyl. The heteroaryl is optionally substituted with one or more (e.g., 1-3, such as 1, 2, or 3) substituents optionally selected from the group consisting of: halogen (e.g., fluorine, chlorine, bromine, or iodine), hydroxy, cyano, amino, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), C₁₋₆ alkoxy (e.g., C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), and any combination thereof.

In some embodiments, PBM represents the structure of Formula (PBM-12-1), Formula (PBM-12-2), or Formula (PBM-12-3):

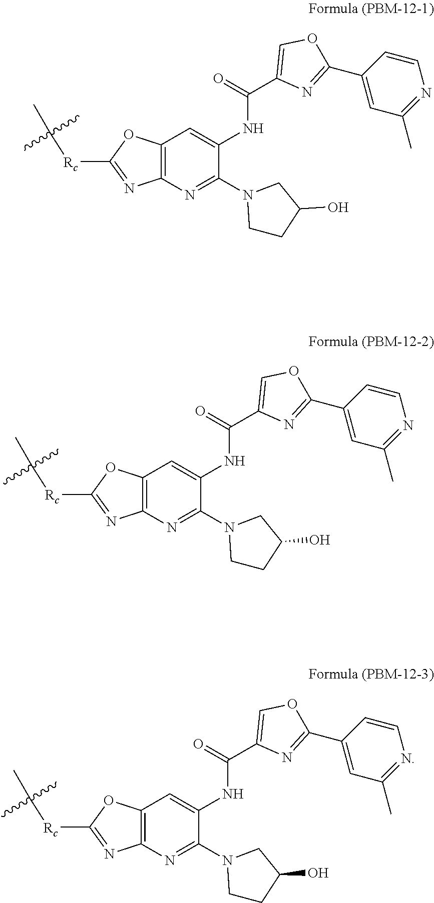

In some embodiments, PBM represents the structure of Formula (PBM-20):

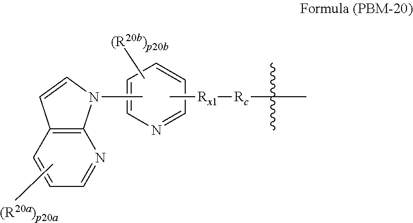

wherein Rₓ₁ in Formula (PBM-20) represents a bond or C(O), or Rₓ₁ represents-C(O)NH—Rₓ₂—C(O)—***, wherein symbol *** indicates the point of attachment to R_{c}, and Rₓ₂ represents optionally substituted C₃₋₁₅ cycloalkyl;
(R^{20a})ₚ₂₀ₐ indicates that the 1H-pyrrolo[2,3-b]pyridine ring to which it is attached is optionally substituted with p20a R^{20a}, wherein each R^{20a} is independently cyano, halogen (e.g., fluorine, chlorine, bromine, or iodine), hydroxy, amino, nitro, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), C₁₋₆ alkoxy (e.g., C₁₋₄ alkoxy, such as methoxy, CD₃—O—, ethoxy, propoxy, isopropoxy, n-butoxy, isobutoxy, or tert-butoxy), or halogenated C₁₋₆ alkoxy (e.g., halogenated C₁₋₄ alkoxy, such as F₃C—O—, FCH₂—O—, F₂CH—O—, ClCH₂—O—, F₂ClCH—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), and p20a represents an integer of 0, 1, 2, 3, 4, or 5;
(R^{20b})_{p20b} indicates that the pyridine ring to which it is attached is optionally substituted with p20b R^{20b} wherein each R^{20b} is independently cyano, halogen (e.g., fluorine, chlorine, bromine, or iodine), hydroxy, nitro, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), C₁₋₆ alkoxy (e.g., C₁₋₄ alkoxy, such as methoxy, CD₃—O—, ethoxy, propoxy, isopropoxy, n-butoxy, isobutoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, F₂ClCH—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), or NR^{20c}R^{20d}, where R^{20c} and R^{20d} are the same or different and independently represent hydrogen, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), optionally substituted C₃₋₁₅ cycloalkyl, or optionally substituted 4-to 15-membered heterocyclyl, and p20b represents an integer of 0, 1, 2, or 3.

In some embodiments, Rₓ₁ in Formula (PBM-20) represents a bond.

In some embodiments, Rₓ₁ in Formula (PBM-20) represents C(O).

In some embodiments, Rₓ₁ in Formula (PBM-20) represents-C(O)NH—Rₓ₂—C(O)—***, wherein symbol *** indicates the point of attachment to R_{c}, and Rₓ₂ represents optionally substituted C₃₋₁₅ cycloalkyl. In some sub-embodiments, examples of the optionally substituted C₃₋₁₅ cycloalkyl include, but are not limited to, optionally substituted C₃₋₁₁ cycloalkyl and optionally substituted C₃₋₈ cycloalkyl, e.g., optionally substituted cyclopropyl, optionally substituted cyclobutyl, optionally substituted cyclopentyl, optionally substituted cyclohexyl, optionally substituted cycloheptyl, or optionally substituted cyclooctyl. In some embodiments, C₃₋₁₅ cycloalkyl is optionally substituted with one or more (e.g., 1-3, 1, 2, or 3) substituents selected from the group consisting of: deuterium, hydroxy, amino, mercapto, nitro, halogen (e.g., fluorine, chlorine, bromine, or iodine), cyano, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), C₁₋₆ alkoxy (e.g., C₁₋₄ alkoxy, such as methoxy, CD₃—O—, ethoxy, propoxy, isopropoxy, n-butoxy, isobutoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, F₂ClCH—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), and any combination thereof.

In some embodiments, (R^{20a})ₚ₂₀ₐ indicates that the 1H-pyrrolo[2,3-b]pyridine ring in Formula (PBM-20) to which it is attached is optionally substituted with p20a R^{20a}, wherein each R^{20a} is independently cyano, halogen (e.g., fluorine, chlorine, bromine, or iodine), hydroxy, amino, nitro, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), C₁₋₆ alkoxy (e.g., C₁₋₄ alkoxy, such as methoxy, CD₃—O—, ethoxy, propoxy, isopropoxy, n-butoxy, isobutoxy, or tert-butoxy), or halogenated C₁₋₆ alkoxy (e.g., halogenated C₁₋₄ alkoxy, such as F₃C—O—, FCH₂—O—, F₂CH—O—, ClCH₂—O—, F₂ClC—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), and p20a represents an integer of 0, 1, 2, 3, 4, or 5.

In some embodiments, (R^{20a})ₚ₂₀ₐ indicates that the 1H-pyrrolo[2,3-b]pyridine ring in Formula (PBM-20) to which it is attached is substituted with p20a R^{20a}, wherein p20a represents an integer of 1, and R^{20a} is cyano.

In some embodiments, (R^{20b})_{p20b} indicates that the pyridine ring to which it is attached is optionally substituted with p20b R^{20b}, wherein each R^{20b} is independently cyano, halogen (e.g., fluorine, chlorine, bromine, or iodine), hydroxy, nitro, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), C₁₋₆ alkoxy (e.g., C₁₋₄ alkoxy, such as methoxy, CD₃—O—, ethoxy, propoxy, isopropoxy, n-butoxy, isobutoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, F₂ClCH—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), or NR^{20c}R^{20d}, where R^{20c} and R^{20d} are the same or different and independently represent hydrogen, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), optionally substituted C₃₋₁₅ cycloalkyl (e.g., optionally substituted C₃₋₁₂ cycloalkyl, and optionally substituted C₃₋₈ cycloalkyl), or optionally substituted 4-to 15-membered heterocyclyl (e.g., optionally substituted 4-to 12-membered heterocyclyl, or optionally substituted 4-to 10-membered heterocyclyl), and p20b represents an integer of 0, 1, 2, or 3. In some sub-embodiments, examples of the optionally substituted C₃₋₁₅ cycloalkyl include, but are not limited to, optionally substituted cyclopropyl, optionally substituted cyclobutyl, optionally substituted cyclopentyl, optionally substituted cyclohexyl, optionally substituted cycloheptyl, or optionally substituted cyclooctyl. In some sub-embodiments, examples of the optionally substituted 4-to 15-membered heterocyclyl include, but are not limited to, azetidinyl, oxetanyl, pyrrolidinyl, imidazolidinyl, pyrazolidyl, tetrahydrofuranyl, tetrahydropyranyl, tetrahydrothienyl, tetrahydrothiopyranyl, oxazolidinyl, thiazolidinyl, piperidinyl, piperazinyl, morpholinyl, thiomorpholinyl, dioxacyclohexyl, azacycloheptyl, diazacycloheptanyl, azacyclooctyl, and diazacyclooctyl. In some sub-embodiments, substituents of the optionally substituted C₃₋₁₅ cycloalkyl and optionally substituted 4-to 15-membered heterocyclyl are optionally selected from the group consisting of: deuterium, hydroxy, amino, mercapto, nitro, halogen (e.g., fluorine, chlorine, bromine, or iodine), cyano, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), C₁₋₆ alkoxy (e.g., C₁₋₄ alkoxy, such as methoxy, CD₃—O—, ethoxy, propoxy, isopropoxy, n-butoxy, isobutoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, F₂ClC—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), and any combination thereof.

In some embodiments, (R^{20b})_{p20b} in Formula (PBM-20) indicates that the pyridine ring to which it is attached is substituted with p20b R^{20b}, wherein p20b represents an integer of 1, and R^{20b} is NR^{20c}R^{20d}, wherein R^{20c} represents hydrogen, and R^{20d} represents C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl).

In some embodiments, PBM represents the structure of Formula (PBM-20-1), Formula (PBM-20-2), Formula (PBM-20-3), or Formula (PBM-20-4):

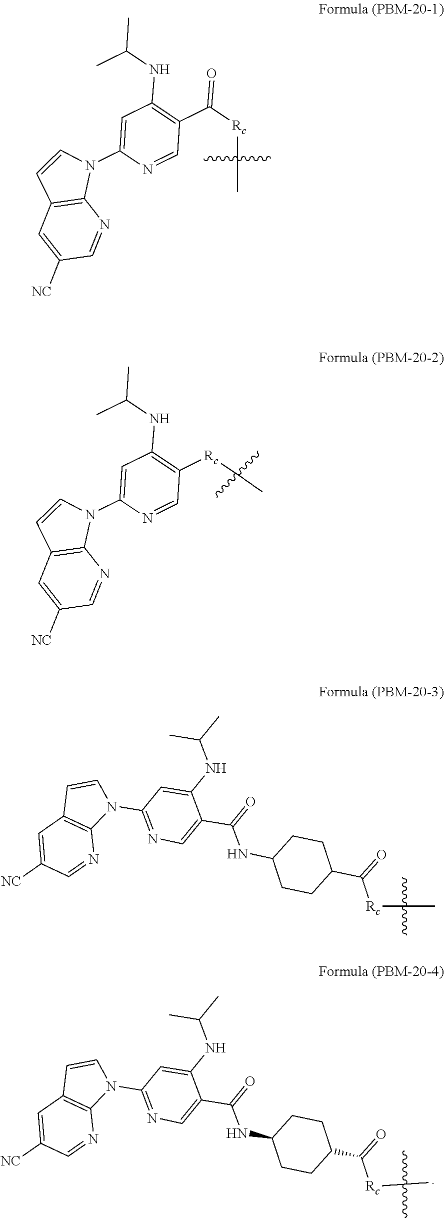

In some embodiments, PBM represents the structure of Formula (PBM-23):

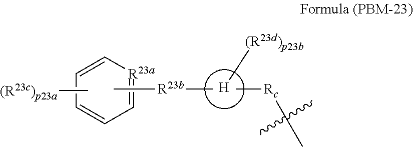

wherein R^{23a} in Formula (PBM-23) represents N or CH;
R^{23b} represents NHC(O), C(O)NH, NHS(O)₂, or S(O)₂NH;
(R^{23c})ₚ₂₃ₐ indicates that the 6-membered ring containing R^{23a} is substituted with p23a R^{23c}, wherein each R^{23c} is the same or different and independently represents halogen (e.g., fluorine, chlorine, bromine, or iodine), hydroxy, mercapto, cyano, amino, nitro, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), C₁₋₆ alkoxy (e.g., C₁₋₄ alkoxy, such as methoxy, CD₃—O—, ethoxy, propoxy, isopropoxy, n-butoxy, isobutoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, F₂ClCH—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), and p23a represents an integer of 0, 1, 2, 3, or 4, and ring H represents 5-to 15-membered heteroarylene, and (R^{23d})_{p23b} indicates that the ring H to which it is attached is optionally substituted with p23b R^{23d}, wherein each R^{23d} is the same or different and independently represents halogen (e.g., fluorine, chlorine, bromine, or iodine), hydroxy, mercapto, cyano, amino, nitro, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), hydroxy-substituted C₁₋₆ alkyl, amino-substituted C₁₋₆ alkyl, C₁₋₆ alkoxy (e.g., C₁₋₄ alkoxy, such as methoxy, CD₃—O—, ethoxy, propoxy, isopropoxy, n-butoxy, isobutoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, F₂ClCH—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), or optionally substituted C₃₋₁₅ cycloalkyl, and p23b represents an integer of 0, 1, 2, or 3.

In some embodiments, R^{23a} represents N. In some embodiments, R^{23a} represents CH.

In some embodiments, (R^{23c})ₚ₂₃ₐ indicates that the 6-membered ring containing R^{23a} is substituted with p23a R^{23c}, wherein each R^{23c} is the same or different and independently represents halogen (e.g., fluorine, chlorine, bromine, or iodine), hydroxy, mercapto, cyano, amino, nitro, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), deuterated C₁₋₆ alkyl (e.g., deuterated C₁₋₃ alkyl, such as CD₃), C₁₋₆ alkoxy (e.g., C₁₋₄ alkoxy, such as methoxy, ethoxy, propoxy, isopropoxy, n-butoxy, sec-butoxy, or tert-butoxy), or halogenated C₁₋₆ alkoxy (e.g., halogenated C₁₋₄ alkoxy, such as F₃C—O—, FCH₂—O—, F₂CH—O—, ClCH₂—O—, F₂ClC—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), and p23a represents an integer of 0, 1, 2, 3, or 4, and the aromatic ring containing R^{23a} is a pyridine ring or a benzene ring. In some embodiments, the aromatic ring containing R^{23a} is a pyridine ring, and R^{23c} represents F₃C—, and p23a represents an integer of 1.

In some embodiments, ring H represents 5-to 15-membered heteroarylene, e.g., 5-to 12-membered, or 5-to 10-membered heteroarylene. In some embodiments, examples of ring H include, but are not limited to, benzimidazolylene, benzothiazolylene, 2H-indazolylene, benzoxazolylene, isoindolylene, benzofuranylene, benzisoxazolylene, isobenzofuranylene, benzothienylene, benzisothiazolylene, benzotriazolylene, benzo[2,1,3]oxadiazolylene, benzo[2,1,3]thiadiazolylene, benzo[1,2,3]thiadiazolylene, quinolinylene, isoquinolinylene, naphthyridinylene, cinnolinylene, quinazolinylene, quinoxalinylene, phthalazinylene, pyrazolo[1,5-a]pyridylene, pyrazolo[1,5-a]pyrimidinylene, imidazo[1,2-a]pyridylene, 1H-pyrrolo[3,2-b]pyridylene, 1H-pyrrolo[2,3-b]pyridylene, pyrrolo[2,1-b]thiazolylene, imidazo[2,1-b]thiazolylene, imidazo[1,2-b]pyridazinylene, and 6,7-dihydro-5H-pyrrolo[1,2-c]imidazolylene. Ring His optionally substituted with p23b R^{23d}, wherein each R^{23d} is the same or different and independently represents halogen (e.g., fluorine, chlorine, bromine, or iodine), hydroxy, mercapto, cyano, amino, nitro, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), deuterated C₁₋₆ alkyl (e.g., deuterated C₁₋₃ alkyl, such as CD₃), hydroxy-substituted C₁₋₆ alkyl (e.g., hydroxy-substituted C₁₋₄ alkyl, such as hydroxypropyl, and hydroxyisopropyl), amino-substituted C₁₋₆ alkyl (e.g., amino-substituted C₁₋₄ alkyl, such as aminopropyl, aminoisopropyl), C₁₋₆ alkoxy (e.g., C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, F₂ClCH—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), or optionally substituted C₃₋₁₅ cycloalkyl, and p23b represents an integer of 0, 1, 2, or 3. In some embodiments, R^{23d} represents hydroxypropyl or hydroxyisopropyl, and p23b represents an integer of 1. In some embodiments, R^{23d} represents optionally substituted C₃₋₁₅ cycloalkyl. Examples of C₃₋₁₅ cycloalkyl include, but are not limited to, cyclopropyl, cyclobutyl, cyclopentyl, cyclopentenyl, cyclohexyl, cyclohexenyl, cycloheptyl, cyclooctyl, decalinyl, octahydropentalenyl, octahydro-1H-indenyl, 2,3-dihydro-1H-indenyl, spiro-cycloalkyl (e.g., C₅₋₁₅ spiro-cycloalkyl), adamantanyl, noradamantanyl, and norbornyl. The optional substituents of C₃₋₁₅ cycloalkyl are optionally selected from the group consisting of halogen (e.g., fluorine, chlorine, bromine, or iodine), hydroxy, mercapto, cyano, amino, nitro, C₁₋₆ alkyl (e.g., C₁₋₄ alkyl, or C₁₋₃ alkyl, such as methyl, ethyl, propyl, isopropyl, butyl, sec-butyl, tert-butyl, pentyl, or hexyl), halogenated C₁₋₆ alkyl (e.g., halogenated C₁₋₄ alkyl, or halogenated C₁₋₃ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), C₁₋₆ alkoxy (e.g., C₁₋₄ alkoxy, such as methoxy, CD₃—O—, ethoxy, propoxy, isopropoxy, n-butoxy, isobutoxy, or tert-butoxy), and halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—).

In some embodiments, PBM represents a structure of Formula (PBM-23-1), Formula (PBM-23-2), Formula (PBM-23-3), Formula (PBM-23-4), Formula (PBM-23-5), Formula (PBM-23-6), Formula (PBM-23-7), Formula (PBM-23-8), Formula (PBM-23-9), Formula (PBM-23-10), Formula (PBM-23-11), Formula (PBM-23-12), or Formula (PBM-23-13):

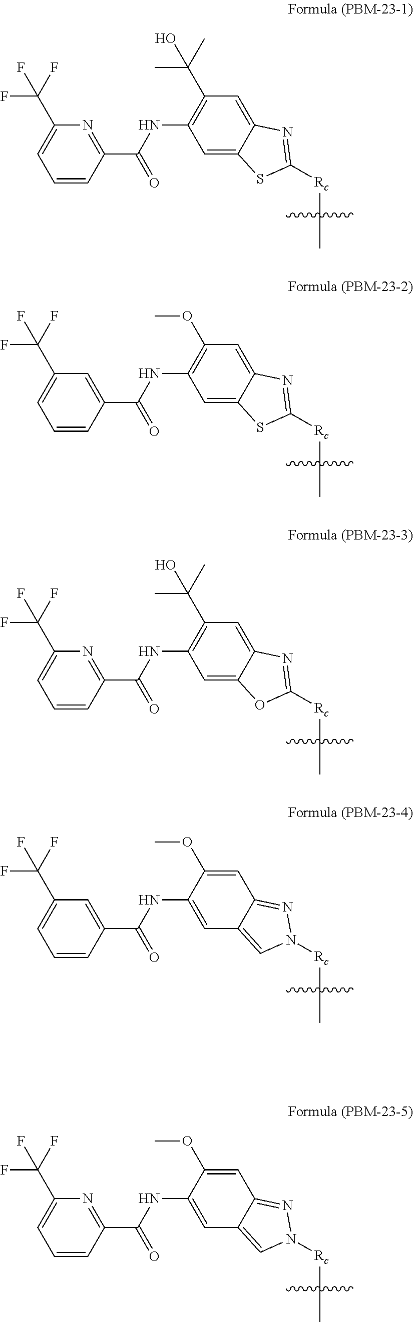

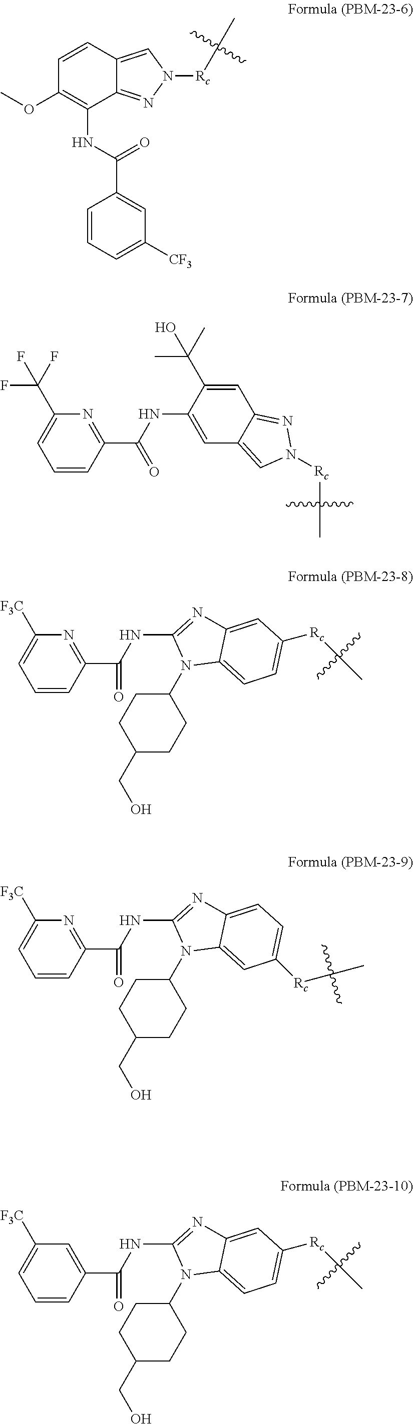

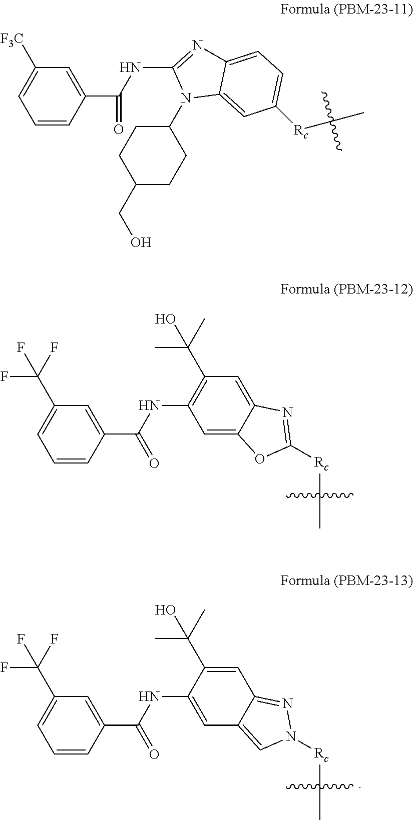

In some embodiments, PBM represents a small molecule ligand of CDK9.

In some embodiments, PBM represents a structure of Formula (PBM-13):

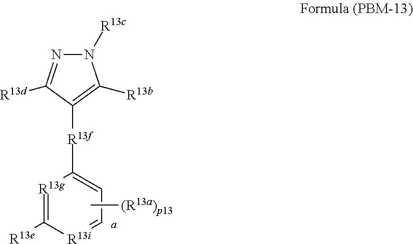

wherein R^{13g} in Formula (PBM-13) represents N or CR^{13h}, where R^{13h} represents hydrogen or halogen (e.g., fluorine, chlorine, bromine, or iodine); R¹³ⁱ represents N or CR^{13j}, where R^{13j} represents hydrogen or halogen (e.g., fluorine, chlorine, bromine, or iodine);
R^{13f} represents a bond, —C(O)NH—, —NHC(O)—, —S(O)₂NH—, or —NHS(O)₂—;
(R^{13a})ₚ₁₃ₐ indicates that the 6-membered ring containing R^{13g} and R¹³ⁱ in Formula (PBM-13) is optionally substituted with p13a R^{13a}, with each R^{13a} being the same or different and independently representing deuterium, halogen (e.g., fluorine, chlorine, bromine, or iodine), hydroxy, cyano, amino, C₁₋₆ alkyl (e.g., C₁₋₄ alkyl, or C₁₋₃ alkyl, such as methyl, ethyl, propyl, isopropyl, butyl, sec-butyl, tert-butyl, pentyl, or hexyl), halogenated C₁₋₆ alkyl (e.g., halogenated C₁₋₄ alkyl, or halogenated C₁₋₃ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), C₁₋₆ alkoxy (e.g., C₁₋₄ alkoxy, such as methoxy, CD₃—O—, ethoxy, propoxy, isopropoxy, n-butoxy, isobutoxy, or tert-butoxy), and halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—);
p13a represents an integer of 0, 1 or 2;
R^{13b} represents hydrogen, halogen (e.g., fluorine, chlorine, bromine, or iodine), C₁₋₆ alkyl (e.g., C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), halogenated C₁₋₆ alkyl (e.g., halogenated C₁₋₄ alkyl, or halogenated C₁₋₃ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), or —C₁₋₃ alkylene-C₃₋₆ cycloalkyl;
R^{13c} represents hydrogen, C₁₋₆ alkyl (e.g., C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), or halogenated C₁₋₆ alkyl (e.g., halogenated C₁₋₄ alkyl, or halogenated C₁₋₃ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), or
R^{13b} and R^{13c} together with their respective attached nitrogen and carbon atoms form an optionally substituted 5-to 7-membered nitrogen-containing heterocycle;
R^{13d} represents hydrogen or R_{c}, and R^{13e} represents hydrogen or R_{c}, wherein R^{13d} and R^{13e} are not simultaneously hydrogen, and only one of R^{13d} and R^{13e} represents R_{c}, and the other represents hydrogen. (In other words, one of R^{13d} and R^{13e} represents R_{c}, and the other represents hydrogen).

In some embodiments, R^{13f} in Formula (PBM-13) represents a bond.

In some embodiments, R^{13f} in Formula (PBM-13) represents-C(O)NH—, —NHC(O)—, —S(O)₂NH—, or—NHS(O)₂—.

In some embodiments, R^{13b} in Formula (PBM-13) represents hydrogen, halogen (e.g., fluorine, chlorine, bromine, or iodine), C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), or —C₁₋₃ alkylene-C₃₋₆ cycloalkyl (e.g.,-methylene-cyclopropyl), and/or
R^{13c} represents hydrogen, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), or halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—).

In some embodiments, R^{13b} and R^{13c} in Formula (PBM-13) together with their respective attached nitrogen and carbon atoms form an optionally substituted 5-to 7-membered nitrogen-containing heterocycle. In some embodiments, the 5-to 7-membered nitrogen-containing heterocycle includes, but is not limited to, e.g., 5-to 7-membered heterocycle (e.g., 5-to 6-membered or 5-membered heterocycle) containing one nitrogen atom and optionally containing heteroatoms selected from nitrogen, oxygen, and sulfur. The 5-to 7-membered nitrogen-containing heterocycle is fused with the pyrazole ring in Formula (PBM-13) to form a fused ring system, including, but not limited to, a 5,6-dihydro-4H-pyrrolo[1,2-b]pyrazole ring. The 5-to 7-membered nitrogen-containing heterocycle is optionally substituted with one or more (e.g., 1 to 6, or 1 to 5, or 1 to 4, 1 to 3, or 2) substituents selected from the group consisting of: halogen (e.g., fluorine, chlorine, bromine, or iodine), hydroxy, amino, cyano, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), or deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.).

In some embodiments, the 5-to 7-membered nitrogen-containing heterocycle, formed by R^{13b} and R^{13c} together with their respective attached nitrogen and carbon atoms, is fused with the adjacent pyrazole ring to form the following groups:

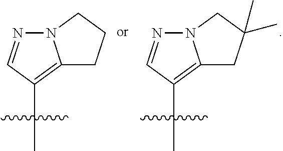 [SMILES: *c1cnn2c1CCC2 ; *c1cnn2c1CC(C)(C)C2]

In some embodiments, PBM represents the structure of Formula (PBM-13-1A):

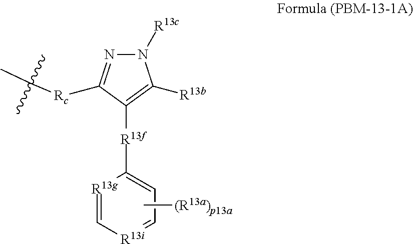

wherein R^{13g} represents N or CR^{13h}, where R^{13h} represents hydrogen or halogen; R¹³ⁱ represents N or CR^{13j}, where R^{13j} represents hydrogen or halogen;
R^{13f} represents a bond, —C(O)NH—, —NHC(O)—, —S(O)₂NH—, or —NHS(O)₂—;
(R^{13a})ₚ₁₃ₐ indicates that the 6-membered ring containing R^{13g} and R¹³ⁱ to which it is attached is optionally substituted with p13a R^{13a}, wherein each R^{13a} represents deuterium, halogen (e.g., fluorine, chlorine, bromine, or iodine), hydroxy, cyano, amino, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), C₁₋₆ alkoxy (e.g., C₁₋₄ alkoxy, such as methoxy, CD₃—O—, ethoxy, propoxy, isopropoxy, n-butoxy, isobutoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), or halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—);
p13a represents an integer of 0, 1 or 2;
R^{13b} represents hydrogen, halogen (e.g., fluorine, chlorine, bromine, or iodine), C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), or —C₁₋₃ alkylene-C₃₋₆ cycloalkyl (e.g.,-methylene-cyclopropyl);
R^{13c} represents hydrogen, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), or halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), or
R^{13b} and R^{13c} together with their respective attached nitrogen and carbon atoms form an optionally substituted 5-to 7-membered nitrogen-containing heterocycle.

In some embodiments, PBM represents the structure of Formula (PBM-13-1B):

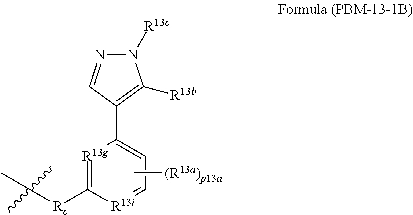

wherein R^{13g} represents N or CR^{13h}, where R^{13h} represents hydrogen or halogen; R¹³ⁱ represents N or CR^{13j}, where R^{13j} represents hydrogen or halogen;
(R^{13a})ₚ₁₃ₐ indicates that the 6-membered ring containing R^{13g} and R¹³ⁱ to which it is attached is optionally substituted with p13a R^{13a}, wherein each R^{13a} represents deuterium, halogen (e.g., fluorine, chlorine, bromine, or iodine), hydroxy, cyano, amino, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), C₁₋₆ alkoxy (e.g., C₁₋₄ alkoxy, such as methoxy, CD₃—O—, ethoxy, propoxy, isopropoxy, n-butoxy, isobutoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), or halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—);
p13a represents an integer of 0, 1 or 2;
R^{13b} represents hydrogen, halogen (e.g., fluorine, chlorine, bromine, or iodine), C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), or—C₁₋₃ alkylene-C₃₋₆ cycloalkyl (e.g.,-methylene-cyclopropyl);
R^{13c} represents hydrogen, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), or halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), or
R^{13b} and R^{13c} together with their respective attached nitrogen and carbon atoms form an optionally substituted 5-to 7-membered nitrogen-containing heterocycle.

In some embodiments, R^{13b} in each of Formula (PBM-13-1A) and Formula (PBM-13-1B) each independently represents hydrogen, halogen (e.g., fluorine, chlorine, bromine, or iodine), C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), or—C₁₋₃ alkylene-C₃₋₆ cycloalkyl (e.g.,-methylene-cyclopropyl).

In some embodiments, R^{13c} in each of Formula (PBM-13-1A) and Formula (PBM-13-1B) each independently represents hydrogen, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), or halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—).

In some embodiments, R^{13b} and R^{13c} in Formula (PBM-13-1A) each independently represent hydrogen.

In some embodiments, R^{13b} in Formula (PBM-13-1B) represents-methylene-cyclopropyl, and R^{13c} represents methyl.

In some embodiments of Formula (PBM-13-1A) and Formula (PBM-13-1B), the pyrazole ring is fused with a nitrogen-containing heterocycle, which is formed by R^{13b} and R^{13c} together with their respective attached nitrogen and carbon atoms, to form the following group:

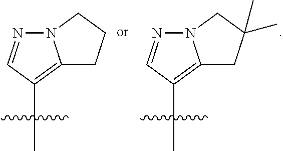 [SMILES: *c1cnn2c1CCC2 ; *c1cnn2c1CC(C)(C)C2]

In some embodiments, PBM represents the structure of Formula (PBM-13-1) or Formula (PBM-13-2):

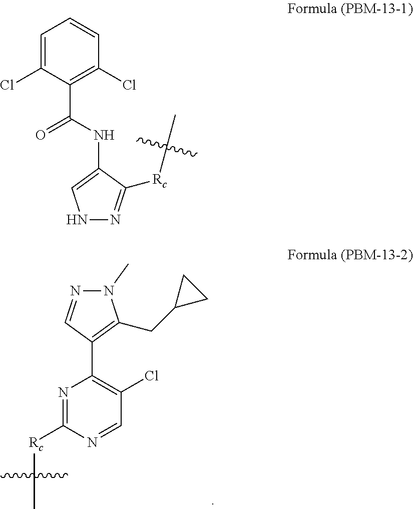

In some embodiments, PBM represents the structure of Formula (PBM-14):

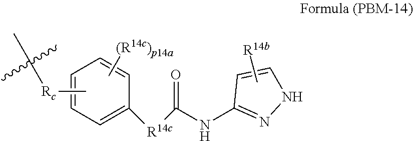

wherein R^{14a} represents a bond, C₁₋₃ alkylene (e.g., methylene, ethylene, or propylene), or halogenated C₁₋₃ alkylene (e.g., halogenated methylene, halogenated ethylene or halogenated propylene);
R^{14b} represents halogen (e.g., fluorine, chlorine, bromine, or iodine), C₁₋₆ alkoxy (e.g., C₁₋₄ alkoxy, such as methoxy, CD₃—O—, ethoxy, propoxy, isopropoxy, n-butoxy, isobutoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), optionally substituted C₃₋₁₅ cycloalkyl (e.g., optionally substituted cyclopropyl, optionally substituted cyclobutyl, optionally substituted cyclopentyl, or optionally substituted cyclohexyl, or e.g., halogenated C₃₋₆ cycloalkyl, such as halogenated cyclopropyl, halogenated cyclobutyl, halogenated cyclopentyl, or halogenated cyclohexyl), or optionally substituted C₁₋₆ alkyl (e.g., optionally substituted C₁₋₅ alkyl, optionally substituted C₁₋₄ alkyl, or optionally substituted C₁₋₃ alkyl);
(R^{14c})ₚ₁₄ₐ indicates that the benzene ring in Formula (PBM-14) is optionally substituted with p14a R^{14c} with each R^{14c} being the same or different and independently representing deuterium, halogen, hydroxy, cyano, amino, C₁₋₆ alkyl, C₁₋₆ alkoxy, halogenated C₁₋₆ alkoxy, deuterated C₁₋₆ alkyl, or halogenated C₁₋₆ alkyl, and
p14a represents an integer of 0, 1, 2, 3, or 4.

In some embodiments, R^{14a} in Formula (PBM-14) represents a bond.

In some embodiments, R^{14a} in Formula (PBM-14) represents C₁₋₃ alkylene (e.g., methylene, ethylene, or propylene), or halogenated C₁₋₃ alkylene (e.g., halogenated methylene, halogenated ethylene or halogenated propylene).

In some embodiments, R^{14b} in Formula (PBM-14) represents optionally substituted C₃₋₁₅ cycloalkyl, e.g., optionally substituted C₃₋₈ cycloalkyl, such as optionally substituted cyclopropyl, optionally substituted cyclobutyl, optionally substituted cyclopentyl, or optionally substituted cyclohexyl. In some embodiments, C₃₋₁₅ cycloalkyl is optionally substituted with one or more (e.g., 1-3, 1, 2, or 3) substituents selected from the group consisting of: deuterium, hydroxy, amino, mercapto, nitro, halogen (e.g., fluorine, chlorine, bromine, or iodine), cyano, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), C₃₋₆ cycloalkyl, C₁₋₆ alkoxy (e.g., C₁₋₄ alkoxy, such as methoxy, CD₃—O—, ethoxy, propoxy, isopropoxy, n-butoxy, isobutoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), and any combination thereof.

In some embodiments, R^{14b} in Formula (PBM-14) represents cyclobutyl.

In some embodiments, PBM represents the structure of Formula (PBM-14-1):

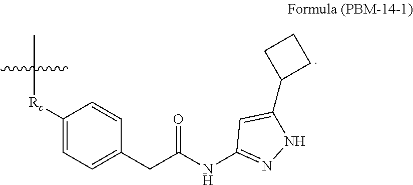

In some embodiments, PBM represents the structure of Formula (PBM-15):

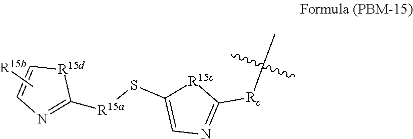

wherein R^{15a} in Formula (PBM-15) represents C₁₋₃ alkylene (e.g., methylene, ethylene, or propylene), or halogenated C₁₋₃ alkylene (e.g., halogenated methylene, halogenated ethylene or halogenated propylene), and R^{15b} represents deuterium, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), or optionally substituted C₃₋₆ cycloalkyl, and
R^{15c} and R^{15d} each independently represent S or O.

In some embodiments, R^{15b} in Formula (PBM-15) represents optionally substituted C₃₋₆ cycloalkyl, e.g., optionally substituted cyclopropyl, optionally substituted cyclobutyl, optionally substituted cyclopentyl, or optionally substituted cyclohexyl. In some embodiments, C₃₋₆ cycloalkyl is optionally substituted with one or more (e.g., 1-3, 1, 2, or 3) substituents selected from the group consisting of: deuterium, hydroxy, amino, mercapto, nitro, cyano, halogen (e.g., fluorine, chlorine, bromine, or iodine), C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), optionally deuterated C₃₋₆ cycloalkyl (e.g., optionally perdeuterated cyclopropyl, optionally perdeuterated cyclobutyl, optionally perdeuterated cyclopentyl, or optionally perdeuterated cyclohexyl), C₁₋₆ alkoxy (e.g., C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), and any combination thereof.

In some embodiments, PBM represents the structure of Formula (PBM-15-1):

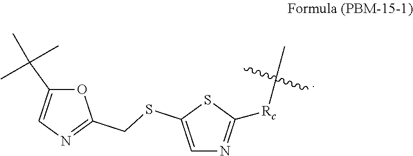

In some embodiments, PBM represents the structure of Formula (PBM-16):

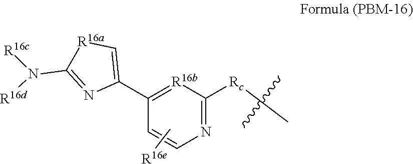

wherein R^{16a} in Formula (PBM-16) represents —S— or a fragment

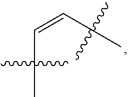 [SMILES: */C=C\*]

wherein when R^{16a} represents —S—, the heteroaryl ring containing R^{16a} and nitrogen atom in Formula (PBM-16) is thiazolyl ring, and when R^{16a} represents the fragment

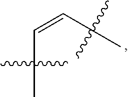 [SMILES: */C=C\*]

the heteroaryl ring containing R^{16a} and nitrogen atom in Formula (PBM-16) is pyridyl ring;
R^{16b} represents N or CH;
R^{16c} represents hydrogen or optionally substituted C₁₋₁₀ alkyl;
R^{16d} represents hydrogen, —C₁₋₃ alkylene-optionally substituted 4-to 10-membered heterocyclyl, or optionally substituted 4-to 10-membered heterocyclyl; and
R^{16e} represents hydroxy, halogen (e.g., fluorine, chlorine, bromine, or iodine), C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), C₁₋₆ alkoxy (e.g., C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), or halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—).

In some embodiments, when R^{16a} in Formula (PBM-16) represents —S—, the heteroaryl ring containing R^{16a} and nitrogen atom in Formula (PBM-16) is thiazolyl.

In some embodiments, when R^{16a} in Formula (PBM-16) represents the fragment

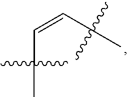 [SMILES: */C=C\*]

the heteroaryl ring containing R^{16a} and nitrogen atom in Formula (PBM-16) is pyridyl.

In some embodiments, R^{16a} in Formula (PBM-16) represents hydrogen or optionally substituted C₁₋₁₀ alkyl. In some embodiments, examples of the optionally substituted C₁₋₁₀ alkyl include, but are not limited to, optionally substituted C₁₋₈ alkyl, optionally substituted C₁₋₆ alkyl, or optionally substituted C₁₋₄ alkyl. In some embodiments, C₁₋₁₀ alkyl is optionally substituted with one or more (e.g., 1-3, 1, 2, or 3) substituents selected from the group consisting of deuterium, hydroxy, amino, mercapto, nitro, halogen (e.g., fluorine, chlorine, bromine, or iodine), cyano, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), C₁₋₆ alkoxy (e.g., C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), or halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), C₃₋₆ cycloalkyl, and any combination thereof.

In some embodiments, R^{16d} in Formula (PBM-16) represents hydrogen, —C₁₋₃ alkylene-optionally substituted 4-to 10-membered heterocyclyl, or optionally substituted 4-to 10-membered heterocyclyl. In some embodiments, each optionally substituted 4-to 10-membered heterocyclyl is independently e.g., 4-to 10-membered (e.g., 4-to 9-membered, 4-to 8-membered, 5-to 7-membered, or 4-to 6-membered) monocyclic or bicyclic heterocyclyl group containing one or more (e.g., 1-4, or 1, 2, 3, or 4) heteroatoms selected from nitrogen, oxygen, and sulfur, including but not limited to azetidinyl, oxetanyl, pyrrolidinyl, imidazolidinyl, pyrazolidyl, tetrahydrofuranyl, tetrahydropyranyl, tetrahydrothienyl, tetrahydrothiopyranyl, oxazolidinyl, thiazolidinyl, piperidinyl, piperazinyl, morpholinyl, thiomorpholinyl, dioxacyclohexyl, azacycloheptyl, diazacycloheptanyl (e.g., 1,4-diazacycloheptan-1-yl), azacyclooctyl, diazacyclooctyl, azabicyclo[3.1.1]heptanyl, azabicyclo[2.2.1]heptanyl, azabicyclo[3.2.1]octanyl, azabicyclo[2.2.2]octanyl, diazabicyclo[3.1.1]heptanyl, diazabicyclo[2.2.1]heptanyl, diazabicyclo[3.2.1]octanyl (e.g., 3,8-diazabicyclo[3.2.1]octan-3-yl), and diazabicyclo[2.2.2]octanyl (e.g., 2,5-diazabicyclo[2.2.2]octan-2-yl). In some embodiments, the 4-to 10-membered heterocyclyl is optionally substituted with a substituent selected from the group consisting of deuterium, hydroxy, amino, mercapto, nitro, halogen (e.g., fluorine, chlorine, bromine, or iodine), cyano, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), C₁₋₆ alkoxy (e.g., C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), or halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), C₃₋₆ cycloalkyl, and any combination thereof.

In some embodiments, PBM represents the structure of Formula (PBM-16-1A) or Formula (PBM-16-1B):

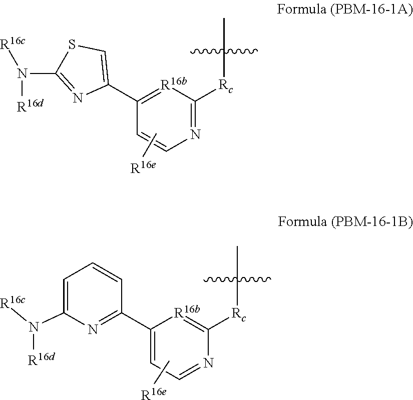

wherein R^{16b} represents N or CH;
R^{16c} represents hydrogen or optionally substituted C₁₋₁₀ alkyl;
R^{16d} represents hydrogen, —C₁₋₃ alkylene-optionally substituted 4-to 10-membered heterocyclyl, or optionally substituted 4-to 10-membered heterocyclyl, and
R^{16e} represents hydroxy, halogen (e.g., fluorine, chlorine, bromine, or iodine), C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), C₁₋₆ alkoxy (e.g., C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), or halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—).

In some embodiments, R^{16c} in each of Formula (PBM-16-1A) and Formula (PBM-16-1B) each independently represents hydrogen or optionally substituted C₁₋₁₀ alkyl. In some embodiments, examples of the optionally substituted C₁₋₁₀ alkyl include, but are not limited to, optionally substituted C₁₋₈ alkyl, optionally substituted C₁₋₆ alkyl, or optionally substituted C₁₋₄ alkyl. In some embodiments, C₁₋₁₀ alkyl is optionally substituted with one or more (e.g., 1-3, 1, 2, or 3) substituents selected from the group consisting of deuterium, hydroxy, amino, mercapto, nitro, halogen (e.g., fluorine, chlorine, bromine, or iodine), cyano, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), C₁₋₆ alkoxy (e.g., C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), or halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), C₃₋₆ cycloalkyl, and any combination thereof.

In some embodiments, R^{16d} in each of Formula (PBM-16-1A) and Formula (PBM-16-1B) each independently represents hydrogen, —C₁₋₃ alkylene-optionally substituted 4-to 10-membered heterocyclyl, or optionally substituted 4-to 10-membered heterocyclyl. In some embodiments, each optionally substituted 4-to 10-membered heterocyclyl is independently e.g., 4-to 10-membered (e.g., 4-to 9-membered, 4-to 8-membered, 5-to 7-membered, or 4-to 6-membered) monocyclic, bicyclic, or polycyclic heterocyclyl group containing one or more (e.g., 1-4, or 1, 2, 3, or 4) heteroatoms selected from nitrogen, oxygen, and sulfur, including but not limited to azetidinyl, oxetanyl, pyrrolidinyl, imidazolidinyl, pyrazolidyl, tetrahydrofuranyl, tetrahydropyranyl, tetrahydrothienyl, tetrahydrothiopyranyl, oxazolidinyl, thiazolidinyl, piperidinyl, piperazinyl, morpholinyl, thiomorpholinyl, dioxacyclohexyl, azacycloheptyl, diazacycloheptanyl (e.g., 1,4-diazacycloheptan-1-yl), azacyclooctyl, diazacyclooctyl, azabicyclo[3.1.1]heptanyl, azabicyclo[2.2.1]heptanyl, azabicyclo[3.2.1]octanyl, azabicyclo[2.2.2]octanyl, diazabicyclo[3.1.1]heptanyl, diazabicyclo[2.2.1]heptanyl, diazabicyclo[3.2.1]octanyl (e.g., 3,8-diazabicyclo[3.2.1]octan-3-yl), and diazabicyclo[2.2.2]octanyl (e.g., 2,5-diazabicyclo[2.2.2]octan-2-yl). In some embodiments, the 4-to 10-membered heterocyclyl is optionally substituted with a substituent selected from the group consisting of deuterium, hydroxy, amino, mercapto, nitro, halogen (e.g., fluorine, chlorine, bromine, or iodine), cyano, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), C₁₋₆ alkoxy (e.g., C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), C₃₋₆ cycloalkyl, and any combination thereof.

In some embodiments, PBM represents a structure of Formula (PBM-16-1) or Formula (PBM-16-2):

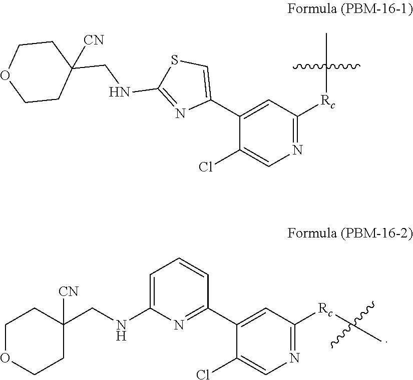

In some embodiments, PBM represents a small molecule ligand of EZH2.

In some embodiments, PBM represents the structure of Formula (PBM-17):

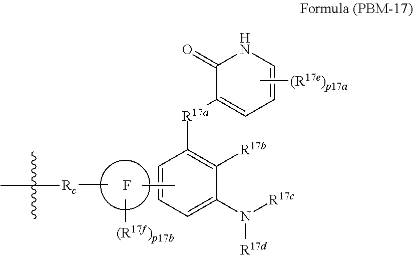

wherein R^{17a} in Formula (PBM-17) represents C(O)NH, NHC(O), (CH₂)₁₋₂C(O)NH, (CH₂)₁₋₂NHC(O), C(O)NH(CH₂)₁₋₂, NHC(O)(CH₂)₁₋₂, S(O)₂NH, or NHS(O)₂;
R^{17b} represents hydrogen, hydroxy, halogen (e.g., fluorine, chlorine, bromine, or iodine), cyano, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), C₁₋₆ alkoxy (e.g., C₁₋₅ alkoxy, C₁₋₄ alkoxy, or C₁₋₃ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), or halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—);
R^{17c} represents hydrogen, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), or halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), or
R^{17b} and R^{17c}, together with their respective attached carbon and nitrogen atoms and the carbon atom on the benzene ring attached to the nitrogen atom, form an optionally substituted 4-to 6-membered heteroaromatic ring or an optionally substituted 4-to 6-membered heterocycle ring;
R^{17d} represents optionally substituted C₃₋₆ cycloalkyl (e.g., optionally substituted cyclopropyl, optionally substituted cyclobutyl, optionally substituted cyclopentyl, or optionally substituted cyclohexyl), C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), or optionally substituted 4-to 20-membered heterocyclyl;
(R^{17e})ₚ₁₇ₐ indicates that pyridin-2 (1H)-one ring to which it is attached is substituted with p17a R^{17e}, with each R^{17e} being the same or different and independently representing hydroxy, halogen (e.g., fluorine, chlorine, bromine, or iodine), cyano, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), C₁₋₆ alkoxy (e.g., C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), or halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—);
p17a represents an integer of 0, 1, 2, or 3;
ring F in Formula (PBM-17) represents arylene or 5-to 20-membered monocyclic or bicyclic heteroarylene containing from 1 to 4 heteroatoms selected from the group consisting of oxygen, nitrogen, and sulfur; and (R^{17f})_{p17b} indicates that the ring F is optionally substituted with p17b R^{17f}, with each R^{17f} being the same or different and each independently representing deuterium, halogen (e.g., fluorine, chlorine, bromine, or iodine), hydroxy, cyano, amino, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), C₁₋₆ alkoxy (e.g., C₁₋₄ alkoxy, such as methoxy, ethoxy, propoxy, isopropoxy, n-butoxy, sec-butoxy, or tert-butoxy), or halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), and
p17b represents an integer of 0, 1, 2, 3, or 4.

In some embodiments, R^{17a} in Formula (PBM-17) represents C(O)NH, NHC(O), CH₂C(O)NH, CH₂CH₂C(O)NH, CH₂NHC(O), CH₂CH₂NHC(O), C(O)NHCH₂, C(O)NHCH₂CH₂, NHC(O)CH₂, NHC(O)CH₂CH₂, S(O)₂NH, or NHS(O)₂.

In some embodiments, R^{17b} in Formula (PBM-17) represents hydrogen, hydroxy, halogen (e.g., fluorine, chlorine, bromine, or iodine), cyano, C₁₋₆ alkoxy (e.g., C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), or halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—).

In some embodiments, R^{17c} in Formula (PBM-17) represents hydrogen, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), or halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—).

In some embodiments, R^{17b} and R^{17c} in Formula (PBM-17), together with their respective attached carbon and nitrogen atoms and the carbon atom on the benzene ring attached to the nitrogen atom, form an optionally substituted 4-to 6-membered heteroaromatic ring (e.g., optionally substituted 4-to 6-membered nitrogen-containing heteroaromatic ring), or optionally substituted 4-to 6-membered heterocycle ring (e.g., optionally substituted 4-to 6-membered nitrogen-containing heterocycle ring). In some embodiments, R^{17b} and R^{17c} in Formula (PBM-17) together represent the following fragment:—CH₂—, —CH═CH—, —CH═N—, —N═CH—, —N═N—, —CH₂—CH₂—, —NH—CH₂—, or —CH₂—CH₂—CH₂—. In some embodiments, the 4-to 6-membered nitrogen-containing heterocycle ring includes, but is not limited to, e.g., 4-to 6-membered (e.g., 4-membered, 5-membered or 6-membered) heterocycle containing one nitrogen atom and optionally containing heteroatoms selected from nitrogen, oxygen, and sulfur. The 4-to 6-membered nitrogen-containing heterocycle is fused with the benzene ring in Formula (PBM-31) to form a fused ring system. The 4-to 6-membered nitrogen-containing heterocycle is optionally substituted with one or more (e.g., 1-4, 1-3, or 2) substituents selected from the group consisting of: halogen (e.g., fluorine, chlorine, bromine, or iodine), hydroxy, amino, cyano, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), or deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.).

In some embodiments, R^{17d} in Formula (PBM-17) represents optionally substituted C₃₋₆ cycloalkyl (e.g., optionally substituted cyclopropyl, optionally substituted cyclobutyl, optionally substituted cyclopentyl, or optionally substituted cyclohexyl), C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), or optionally substituted 4-to 20-membered heterocyclyl.

In some embodiments, R^{17d} in Formula (PBM-17) represents optionally substituted C₃₋₆ cycloalkyl, e.g., optionally substituted cyclopropyl, optionally substituted cyclobutyl, optionally substituted cyclopentyl, or optionally substituted cyclohexyl. In some embodiments, C₃₋₆ cycloalkyl is optionally substituted with one or more (e.g., 1-3, 1, 2, or 3) substituents selected from the group consisting of: deuterium, hydroxy, amino, mercapto, nitro, halogen, cyano, C₁₋₆ alkyl, deuterated C₁₋₆ alkyl, halogenated C₁₋₆ alkyl, C₃-6 cycloalkyl, C₁₋₆ alkoxy, halogenated C₁₋₆ alkoxy, and any combination thereof.

In some embodiments, R^{17d} in Formula (PBM-17) represents optionally substituted 4-to 20-membered heterocyclyl. The optionally substituted 4-to 20-membered heterocyclyl is e.g., optionally substituted 4-to 20-membered (e.g., 4-to 15-membered, 4-to 9-membered, 4-to 8-membered, 5-to 7-membered, or 4-to 6-membered) monocyclic, bicyclic, or polycyclic heterocyclyl group containing one or more (e.g., 1-4, or 1, 2, 3, or 4) heteroatoms selected from nitrogen, oxygen, and sulfur, including but not limited to the following optionally substituted groups: azetidinyl, oxetanyl, pyrrolidinyl, imidazolidinyl, pyrazolidyl, tetrahydrofuranyl, tetrahydropyranyl, tetrahydrothienyl, tetrahydrothiopyranyl, oxazolidinyl, thiazolidinyl, piperidinyl, piperazinyl, morpholinyl, thiomorpholinyl, dioxacyclohexyl, azacycloheptyl, diazacycloheptanyl (e.g., 1,4-diazacycloheptan-1-yl), azacyclooctyl, diazacyclooctyl, azabicyclo[3.1.1]heptanyl, azabicyclo[2.2.1]heptanyl, azabicyclo[3.2.1]octanyl, azabicyclo[2.2.2]octanyl, diazabicyclo[3.1.1]heptanyl, diazabicyclo[2.2.1]heptanyl, diazabicyclo[3.2.1]octanyl (e.g., 3,8-diazabicyclo[3.2.1]octan-3-yl), and diazabicyclo[2.2.2]octanyl (e.g., 2,5-diazabicyclo[2.2.2]octan-2-yl). In some embodiments, the 4-to 20-membered heterocyclyl is optionally substituted with a substituent selected from the group consisting of deuterium, hydroxy, amino, mercapto, nitro, halogen, cyano, C₁₋₆ alkyl, deuterated C₁₋₆ alkyl, halogenated C₁₋₆ alkyl, C₃-6 cycloalkyl, C₁₋₆ alkoxy, halogenated C₁₋₆ alkoxy, and any combination thereof.

In some embodiments, ring F in Formula (PBM-17) represents arylene (e.g., C₅₋₂₀ arylene, C₅₋₁₅ arylene, C₅₋₁₀ arylene, or C₅₋₆ arylene), or 5-to 20-membered (e.g., 5-to 15-membered, 5-to 10-membered, 5-to 9-membered, 5-to 8-membered, 5-to 7-membered, or 6-membered) monocyclic or bicyclic heteroarylene group containing from 1 to 4 (e.g., from 1 to 3, or from 1 to 2) heteroatoms selected from N, O, and S. Examples of the arylene include, but are not limited to, phenylene and naphthylene. Examples of the heteroarylene include, but are not limited to, furanylene, oxazolylene, isoxazolylene, oxadiazolylene, thienylene, thiazolylene, isothiazolylene, thiadiazolylene, pyrrolylene, imidazolylene, pyrazolylene, triazolylene, pyridylene, pyrimidinylene, pyridazinylene, pyrazinylene, indolylene, isoindolylene, benzofuranylene, isobenzofuranylene, benzothienylene, indazolylene, benzimidazolylene, benzoxazolylene, benzisoxazolylene, benzothiazolylene, benzisothiazolylene, benzotriazolylene, benzo[2,1,3]oxadiazolylene, benzo[2,1,3]thiadiazolylene, benzo[1,2,3]thiadiazolylene, quinolinylene, isoquinolinylene, naphthyridinylene, cinnolinylene, quinazolinylene, quinoxalinylene, phthalazinylene, pyrazolo[1,5-a]pyridylene, pyrazolo[1,5-a]pyrimidinylene, imidazo[1,2-alpyridylene, 1H-pyrrolo[3,2-b]pyridylene, 1H-pyrrolo[2,3-b]pyridylene, 4H-furo[3,2-b]pyrrolylene, pyrrolo[2,1-b]thiazolylene, and imidazo[2,1-b]thiazolylene. In some embodiments, the ring F is optionally substituted with p17b R^{17f}, with each R^{17f} being the same or different and independently representing deuterium, halogen (e.g., fluorine, chlorine, bromine, or iodine), hydroxy, cyano, amino, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), C₁₋₆ alkoxy (e.g., C₁₋₄ alkoxy, such as methoxy, ethoxy, propoxy, isopropoxy, n-butoxy, sec-butoxy, or tert-butoxy), or halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—); and p17b represents an integer of 0, 1, 2, 3, or 4.

In some embodiments, PBM represents the structure of Formula (PBM-17-1A), Formula (PBM-17-1B), Formula (PBM-17-1C), Formula (PBM-17-1D), Formula (PBM-17-1E), Formula (PBM-17-1F), Formula (PBM-17-1G), Formula (PBM-17-1H), Formula (PBM-17-1I), Formula (PBM-17-1J), or Formula (PBM-17-1K):

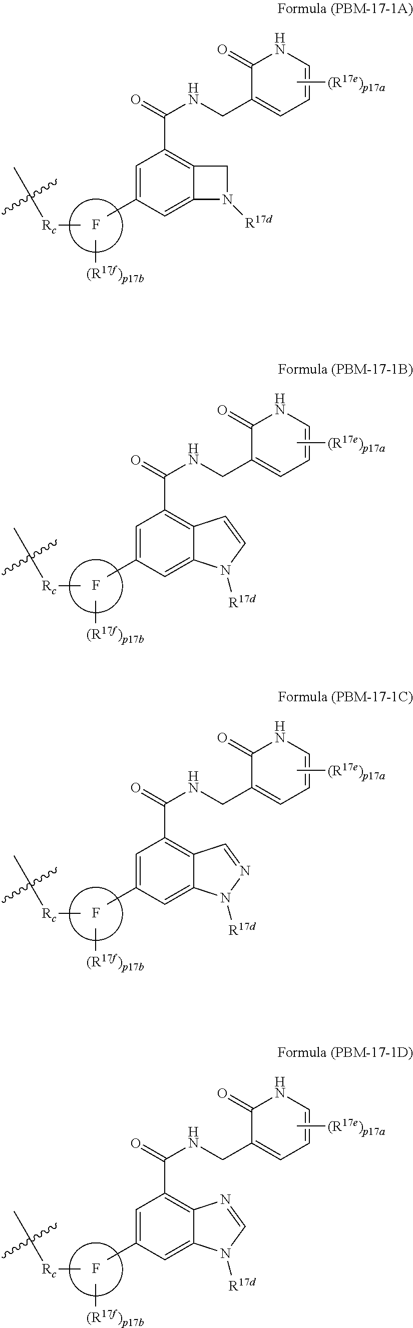

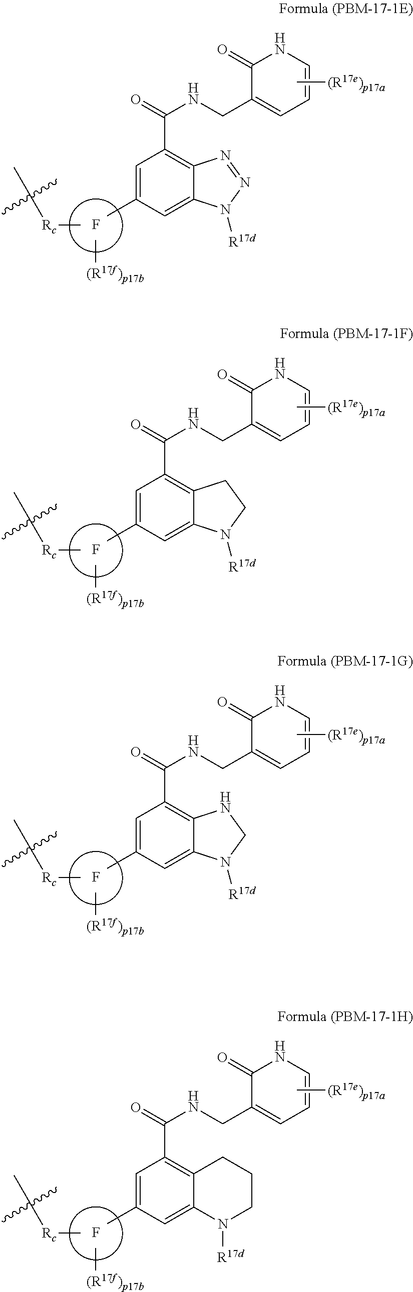

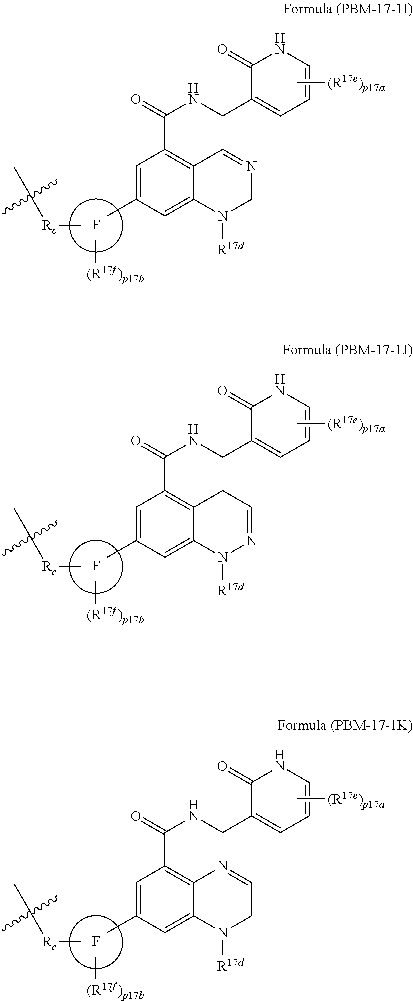

wherein R^{17d} in each of the above formulae independently represents optionally substituted C₃₋₆ cycloalkyl (e.g., optionally substituted cyclopropyl, optionally substituted cyclobutyl, optionally substituted cyclopentyl, or optionally substituted cyclohexyl), C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), or optionally substituted 4-to 10-membered heterocyclyl;
(R^{17e})ₚ₁₇ₐ in each of the above formulae independently represents indicates that pyridin-2 (1H)-one ring to which it is attached is substituted with p17a R^{17e}, wherein each R^{17e} is the same or different and independently represents hydroxy, halogen (e.g., fluorine, chlorine, bromine, or iodine), cyano, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), C₁₋₆ alkoxy (e.g., C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), or halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—);
p17a in each of the above formulae independently represents an integer of 0, 1, 2, or 3;
ring F in each of the above formulae independently represents arylene, or 5-to 20-membered monocyclic or bicyclic heteroarylene containing from 1 to 4 heteroatoms selected from the group consisting of oxygen, nitrogen, and sulfur; and (R^{17f})_{p17b} indicates that the ring F is optionally substituted with p17b R^{17f}, with each R^{17f} being the same or different and each independently representing deuterium, halogen (e.g., fluorine, chlorine, bromine, or iodine), hydroxy, cyano, amino, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), C₁₋₆ alkoxy (e.g., C₁₋₄ alkoxy, such as methoxy, ethoxy, propoxy, isopropoxy, n-butoxy, sec-butoxy, or tert-butoxy), or halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), and
p17b in each of the above formulae independently represents an integer of 0, 1, 2, 3, or 4.

In some embodiments, each R^{17d} in Formula (PBM-17-1A), Formula (PBM-17-1B), Formula (PBM-17-1C), Formula (PBM-17-1D), Formula (PBM-17-1E), Formula (PBM-17-1F), Formula (PBM-17-1G), Formula (PBM-17-1H), Formula (PBM-17-1I), Formula (PBM-17-1J), and Formula (PBM-17-1K) independently represents optionally substituted C₃₋₆ cycloalkyl, e.g., optionally substituted cyclopropyl, optionally substituted cyclobutyl, optionally substituted cyclopentyl, or optionally substituted cyclohexyl. In some embodiments, C₃₋₆ cycloalkyl is optionally substituted with one or more (e.g., 1-3, 1, 2, or 3) substituents selected from the group consisting of: deuterium, hydroxy, amino, mercapto, nitro, halogen, cyano, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), C₁₋₆ alkoxy (e.g., C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), C₃₋₆ cycloalkyl, and any combination thereof.

In some embodiments, each R^{17d} in Formula (PBM-17-1A), Formula (PBM-17-1B), Formula (PBM-17-1C), Formula (PBM-17-1D), Formula (PBM-17-1E), Formula (PBM-17-1F), Formula (PBM-17-1G), Formula (PBM-17-1H), Formula (PBM-17-1I), Formula (PBM-17-1J), and Formula (PBM-17-1K) each independently represents optionally substituted 4-to 20-membered heterocyclyl. The optionally substituted 4-to 20-membered heterocyclyl is e.g., optionally substituted4-to 20-membered (e.g., 4-to 15-membered, 4-to 12-membered, 4-to 9-membered, 4-to 8-membered, 5-to 7-membered, or 4-to 6-membered) monocyclic, bicyclic, or polycyclic heterocyclyl group containing one or more (e.g., 1-4, or 1, 2, 3, or 4) heteroatoms selected from nitrogen, oxygen, and sulfur, including but not limited to the following optionally substituted groups: azetidinyl, oxetanyl, pyrrolidinyl, imidazolidinyl, pyrazolidyl, tetrahydrofuranyl, tetrahydropyranyl, tetrahydrothienyl, tetrahydrothiopyranyl, oxazolidinyl, thiazolidinyl, piperidinyl, piperazinyl, morpholinyl, thiomorpholinyl, dioxacyclohexyl, azacycloheptyl, diazacycloheptanyl (e.g., 1,4-diazacycloheptan-1-yl), azacyclooctyl, diazacyclooctyl, azabicyclo[3.1.1]heptanyl, azabicyclo[2.2.1]heptanyl, azabicyclo[3.2.1]octanyl, azabicyclo[2.2.2]octanyl, diazabicyclo[3.1.1]heptanyl, diazabicyclo[2.2.1]heptanyl, diazabicyclo[3.2.1]octanyl (e.g., 3,8-diazabicyclo[3.2.1]octan-3-yl), and diazabicyclo[2.2.2]octanyl (e.g., 2,5-diazabicyclo[2.2.2]octan-2-yl). In some embodiments, the 4-to 20-membered heterocyclyl is optionally substituted with a substituent selected from the group consisting of deuterium, hydroxy, amino, mercapto, nitro, halogen, cyano, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), C₁₋₆ alkoxy (e.g., C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), C₃₋₆ cycloalkyl, and any combination thereof.

In some embodiments, ring F in each of Formula (PBM-17-1A), Formula (PBM-17-1B), Formula (PBM-17-1C), Formula (PBM-17-1D), Formula (PBM-17-1E), Formula (PBM-17-1F), Formula (PBM-17-1G), Formula (PBM-17-1H), Formula (PBM-17-1I), Formula (PBM-17-1J), and Formula (PBM-17-1K) each independently represents arylene (e.g., C₅₋₂₀ arylene, C₅₋₁₅ arylene, C₅₋₁₀ arylene, or C₅₋₆ arylene), or 5-to 20-membered (e.g., 5-to 15-membered, 5-to 10-membered, 5-to 9-membered, 5-to 8-membered, 5-to 7-membered, or 6-membered) monocyclic or bicyclic heteroarylene group containing from 1 to 4 (e.g., from 1 to 3, or from 1 to 2) heteroatoms selected from N, O, and S. Examples of the arylene include, but are not limited to, phenylene and naphthylene. Examples of heteroarylene include, but are not limited to, furanylene, oxazolylene, isoxazolylene, oxadiazolylene, thienylene, thiazolylene, isothiazolylene, thiadiazolylene, pyrrolylene, imidazolylene, pyrazolylene, triazolylene, pyridylene, pyrimidinylene, pyridazinylene, pyrazinylene, indolylene, isoindolylene, benzofuranylene, isobenzofuranylene, benzothienylene, indazolylene, benzimidazolylene, benzoxazolylene, benzisoxazolylene, benzothiazolylene, benzisothiazolylene, benzotriazolylene, benzo[2,1,3]oxadiazolylene, benzo[2,1,3]thiadiazolylene, benzo[1,2,3]thiadiazolylene, quinolinylene, isoquinolinylene, naphthyridinylene, cinnolinylene, quinazolinylene, quinoxalinylene, phthalazinylene, pyrazolo[1,5-a]pyridylene, pyrazolo[1,5-a]pyrimidinylene, imidazo[1,2-a]pyridylene, 1H-pyrrolo[3,2-b]pyridylene, 1H-pyrrolo[2,3-b]pyridylene, 4H-furo[3,2-b]pyrrolylene, pyrrolo[2,1-b]thiazolylene, and imidazo[2,1-b]thiazolylene. In some embodiments, the ring F is optionally substituted with p17b R^{17f}, and each R^{17f} is the same or different and independently represents deuterium, halogen (e.g., fluorine, chlorine, bromine, or iodine), hydroxy, cyano, amino, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), C₁₋₆ alkoxy (e.g., C₁₋₄ alkoxy, such as methoxy, ethoxy, propoxy, isopropoxy, n-butoxy, sec-butoxy, or tert-butoxy), or halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—); and p17b represents an integer of 0, 1, 2, 3, or 4.

In some embodiments, PBM represents a structure of Formula (PBM-17-1), Formula (PBM-17-2), or Formula (PBM-17-3):

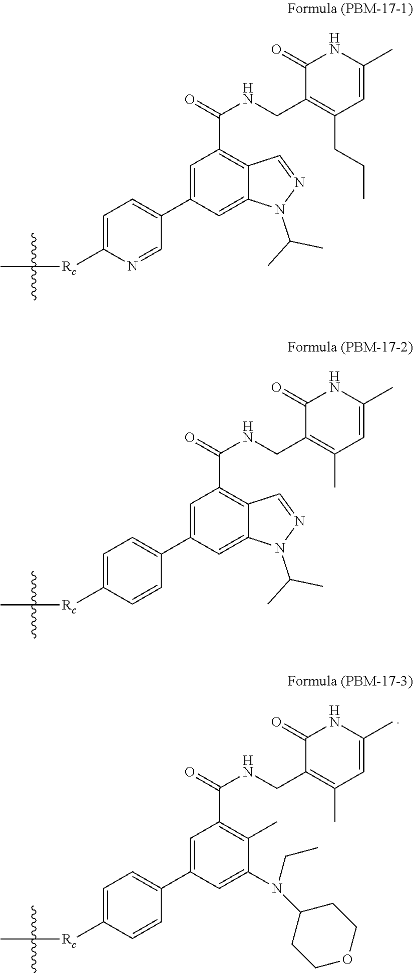

In some embodiments, PBM represents a small molecule ligand of BCL-2.

In some embodiments, PBM represents the structure of Formula (PBM-24):

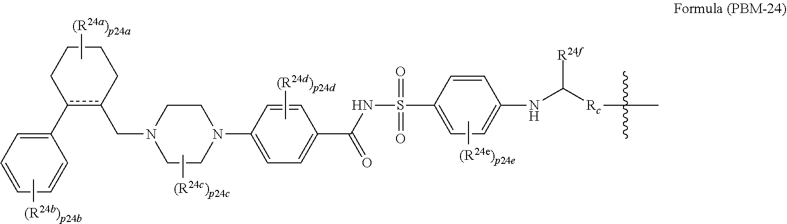

wherein the dashed line-in Formula (PBM-24) indicates the optional presence of a double bond;
(R^{24a})ₚ₂₄ₐ indicates that the 6-membered cycloalkyl ring to which it is attached is optionally substituted with p24a R^{24a}, wherein each R^{24a} is the same or different and independently represents halogen (e.g., fluorine, chlorine, bromine, or iodine), hydroxy, mercapto, cyano, amino, nitro, oxo, C₁₋₆ alkyl (e.g., C₁₋₄ alkyl, such as methyl, ethyl, propyl, isopropyl, butyl, sec-butyl, tert-butyl, pentyl, or hexyl), C₁₋₆ alkoxy (e.g., C₁₋₅ alkoxy, or C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, or halogenated C₁₋₃ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), deuterated C₁₋₆ alkyl (e.g., perdeuterated C₁₋₆ alkyl, or perdeuterated C₁₋₄ alkyl, such as CD₃, CD₃CD₂—, CD₃CD₂CD₂— etc.), or halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), and p24a represents an integer of 0, 1, 2, 3, 4, 5, 6, 7, or 8;
(R^{24b})_{p24b} indicates that the benzene ring to which it is attached is optionally substituted with p24b R^{24b} wherein each R^{24b} is the same or different and independently represents deuterium, halogen (e.g., fluorine, chlorine, bromine, or iodine), hydroxy, mercapto, cyano, amino, nitro, C₁₋₆ alkyl (e.g., C₁₋₄ alkyl, such as methyl, ethyl, propyl, isopropyl, butyl, sec-butyl, tert-butyl, pentyl, or hexyl), C₁₋₆ alkoxy (e.g., C₁₋₅ alkoxy, or C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, or halogenated C₁₋₃ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), deuterated C₁₋₆ alkyl (e.g., perdeuterated C₁₋₆ alkyl, or perdeuterated C₁₋₄ alkyl, such as CD₃, CD₃CD₂—, CD₃CD₂CD₂— etc.), or halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), and p24b represents an integer of 0, 1, 2, 3, 4, or 5;
(R^{24c})_{p24c} indicates that the piperazinylene to which it is attached is optionally substituted with p24c R^{24c}, wherein each R^{24c} is the same or different and independently represents halogen (e.g., fluorine, chlorine, bromine, or iodine), cyano, oxo, C₁₋₆ alkyl (e.g., C₁₋₄ alkyl, such as methyl, ethyl, propyl, isopropyl, butyl, sec-butyl, tert-butyl, pentyl, or hexyl), C₁₋₆ alkoxy (e.g., C₁₋₅ alkoxy, or C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, or halogenated C₁₋₃ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), deuterated C₁₋₆ alkyl (e.g., perdeuterated C₁₋₆ alkyl, or perdeuterated C₁₋₄ alkyl, such as CD₃, CD₃CD₂—, CD₃CD₂CD₂— etc.), or halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), and p24c represents an integer of 0, 1, 2, 3, 4, 5, 6, 7, or 8;
(R^{24d})ₚ₂₄ₐ indicates that the benzene ring to which it is attached is optionally substituted with p24d R^{24d} wherein each R^{24d} is the same or different and independently represents halogen (e.g., fluorine, chlorine, bromine, or iodine), hydroxy, mercapto, C₁₋₆ alkyl (e.g., C₁₋₄ alkyl, such as methyl, ethyl, propyl, isopropyl, butyl, sec-butyl, tert-butyl, pentyl, or hexyl), C₁₋₆ alkoxy (e.g., C₁₋₅ alkoxy, or C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, or halogenated C₁₋₃ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), deuterated C₁₋₆ alkyl (e.g., perdeuterated C₁₋₆ alkyl, or perdeuterated C₁₋₄ alkyl, such as CD₃, CD₃CD₂—, CD₃CD₂CD₂— etc.), or halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), or

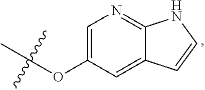 [SMILES: *Oc1cnc2[nH]ccc2c1]

and p24d represents an integer of 0, 1 or 2;
(R^{24e})ₚ₂₄ₑ indicates that the benzene ring to which it is attached is optionally substituted with p24e R^{24e} wherein each R^{24e} is the same or different and independently represents deuterium, halogen (e.g., fluorine, chlorine, bromine, or iodine), hydroxy, mercapto, cyano, amino, nitro, —SO₂CF₃, C₁₋₆ alkyl (e.g., C₁₋₄ alkyl, such as methyl, ethyl, propyl, isopropyl, butyl, sec-butyl, tert-butyl, pentyl, or hexyl), C₁₋₆ alkoxy (e.g., C₁₋₅ alkoxy, or C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, or halogenated C₁₋₃ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), deuterated C₁₋₆ alkyl (e.g., perdeuterated C₁₋₆ alkyl, or perdeuterated C₁₋₄ alkyl, such as CD₃, CD₃CD₂—, CD₃CD₂CD₂— etc.), or halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), and p24e represents an integer of 0, 1, 2, 3, or 4, and
R^{24f} represents hydrogen, deuterium, halogen (e.g., fluorine, chlorine, bromine, or iodine), hydroxy, mercapto, cyano, amino, nitro, C₁₋₆ alkyl (e.g., C₁₋₄ alkyl, such as methyl, ethyl, propyl, isopropyl, butyl, sec-butyl, tert-butyl, pentyl, or hexyl), C₁₋₆ alkoxy (e.g., C₁₋₅ alkoxy, or C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, or halogenated C₁₋₃ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), deuterated C₁₋₆ alkyl (e.g., perdeuterated C₁₋₆ alkyl, or perdeuterated C₁₋₄ alkyl, such as CD₃, CD₃CD₂—, CD₃CD₂CD₂— etc.), or halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), or

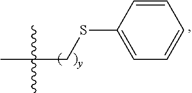

wherein y represents an integer of 1, 2, or 3.

In some sub-embodiments, the dashed line --- in Formula (PBM-24) indicates the presence of a double bond; that is, the six-membered ring group containing this dashed line represents

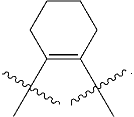 [SMILES: *C1=C(*)CCCC1]

In some sub-embodiments, the dashed line --- in Formula (PBM-24) indicates the absence of a double bond; that is, the six-membered ring group containing this dashed line represents

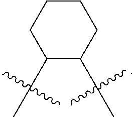 [SMILES: *C1CCCCC1*]

In some sub-embodiments, R^{24d} in Formula (PBM-24) represents

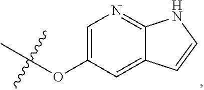 [SMILES: *Oc1cnc2[nH]ccc2c1]

and p24d represents an integer of 1. In some sub-embodiments, p24d represents an integer of 0.

In some sub-embodiments, p24e in Formula (PBM-24) represents an integer of 1, and R^{24e} represents nitro (i.e., NO₂) or SO₂CF₃.

In some sub-embodiments, in Formula (PBM-24), R^{24f} represents hydrogen, or

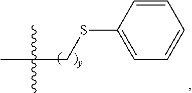

wherein y represents an integer of 1, 2, or 3.

In some sub-embodiments, PBM represents a structure of Formula (PBM-24-1), Formula (PBM-24-2), Formula (PBM-24-3), Formula (PBM-24-4), or Formula (PBM-24-5):

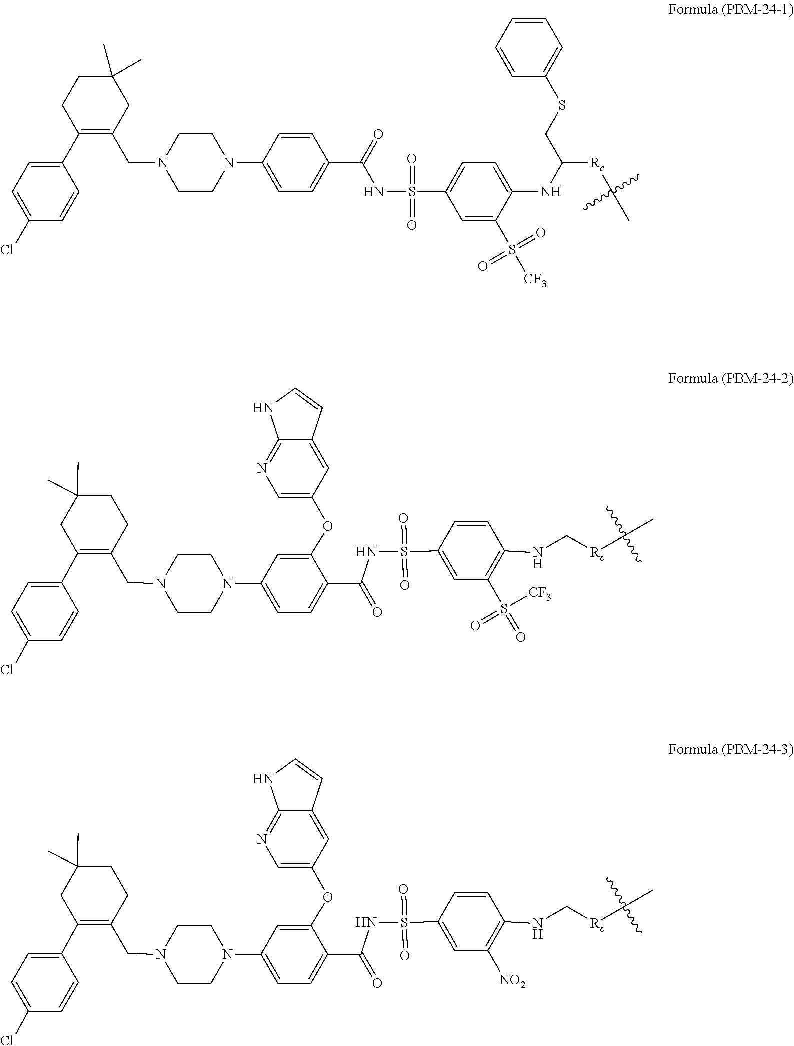

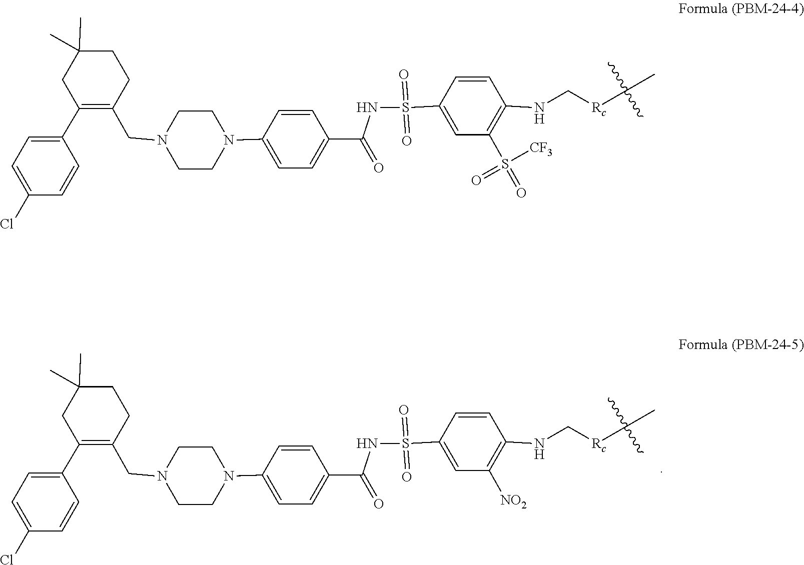

In some embodiments, PBM represents a small molecule ligand of RSK.

In some embodiments, PBM represents a structure of Formula (PBM-25):

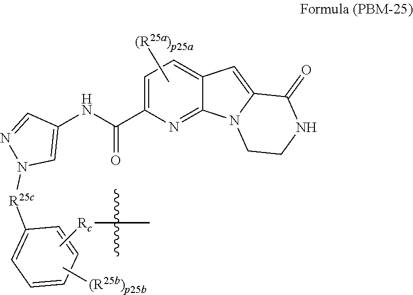

wherein (R^{25a})ₚ₂₅ₐ in Formula (PBM-25) represents p25a R^{25a} groups, wherein each R^{25a} is the same or different and independently represents halogen (e.g., fluorine, chlorine, bromine, or iodine), hydroxy, mercapto, cyano, amino, nitro, C₁₋₆ alkyl (e.g., C₁₋₄ alkyl, such as methyl, ethyl, propyl, isopropyl, butyl, sec-butyl, tert-butyl, pentyl, or hexyl), C₁₋₆ alkoxy (e.g., C₁₋₅ alkoxy, or C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), deuterated C₁₋₆ alkyl (e.g., perdeuterated C₁₋₆ alkyl, or perdeuterated C₁₋₄ alkyl, such as CD₃, CD₃CD₂—, CD₃CD₂CD₂— etc.), or halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), and p25a represents an integer of 0, 1, 2, 3, 4, or 5;
(R^{25b})_{p25b} indicates that the benzene ring to which it is attached is optionally substituted with p25b R^{25b}, wherein each R^{25b} is the same or different and independently represents deuterium, halogen (e.g., fluorine, chlorine, bromine, or iodine), hydroxy, mercapto, cyano, amino, nitro, C₁₋₆ alkyl (e.g., C₁₋₄ alkyl, such as methyl, ethyl, propyl, isopropyl, butyl, sec-butyl, tert-butyl, pentyl, or hexyl), C₁₋₆ alkoxy (e.g., C₁₋₅ alkoxy, or C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), deuterated C₁₋₆ alkyl (e.g., perdeuterated C₁₋₆ alkyl, or perdeuterated C₁₋₄ alkyl, such as CD₃, CD₃CD₂—, CD₃CD₂CD₂— etc.), or halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), and p25b represents an integer of 0, 1, 2, 3, or 4, and
R^{25c} represents C₁₋₆ alkylene or halogenated C₁₋₆ alkylene.

In some embodiments, R^{25c} represents C₁₋₆ alkylene, e.g., methylene or ethylene.

In some embodiments, R^{25c} represents halogenated C₁₋₆ alkylene, e.g., difluoromethylene.

In some embodiments, PBM represents the structure of Formula (PBM-25-1):

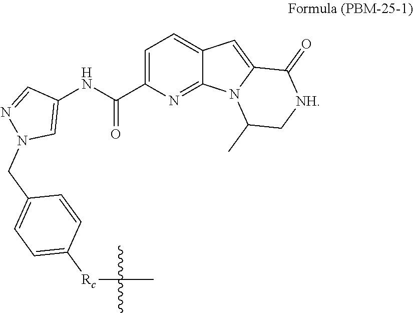

In some embodiments, PBM represents a small molecule ligand of SMARCA2/4.

In some embodiments, PBM represents the structure of Formula (PBM-18-1A)(which is an example of the structure of Formula (PBM-18)):

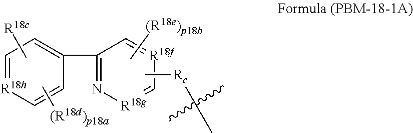

wherein R^{18f}, R^{18g}, and R^{18h} in Formula (PBM-18-1A) each independently represent CH or N;
R^{18c} represents halogen (e.g., fluorine, chlorine, bromine, or iodine), amino, hydroxy, cyano, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), C₁₋₆ alkoxy (e.g., C₁₋₄ alkoxy, or C₁₋₃ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), halogenated C₁₋₆ alkyl (e.g., halogenated C₁₋₅ alkyl, or halogenated C₁₋₃ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), optionally substituted cycloalkyl (e.g., optionally substituted C₃₋₁₅ cycloalkyl), or optionally substituted 4-to 15-membered nitrogen-containing heterocyclyl;
(R^{18d})ₚ₁₈ₐ indicates that the 6-membered ring to which it is attached is optionally substituted with p18a R^{18d}, with each R^{18d} being the same or different and each independently representing hydroxy, amino, cyano, halogen (e.g., fluorine, chlorine, bromine, or iodine), C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), C₁₋₆ alkoxy (e.g., C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), or halogenated C₁₋₆ alkyl (e.g., halogenated C₁₋₅ alkyl, or halogenated C₁₋₃ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—);
p18a represents an integer of 0, 1, 2, 3, or 4;
(R^{18e})_{p18b} indicates that the 6-membered nitrogen-containing heteroaryl ring to which it is attached is optionally substituted with p18b R^{18e}, with each R^{18e} being the same or different and each independently representing hydroxy, amino, cyano, halogen (e.g., fluorine, chlorine, bromine, or iodine), C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), C₁₋₆ alkoxy (e.g., C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), or halogenated C₁₋₆ alkyl (e.g., halogenated C₁₋₅ alkyl, or halogenated C₁₋₃ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), and
p18b represents an integer of 0, 1 or 2.

In some embodiments, R^{18f} in Formula (PBM-18-1A) represents CH, and R^{18g} represents CH. In some embodiments, R^{18f} in Formula (PBM-18-1A) represents CH, and R^{18g} represents N. In some embodiments, R^{18f} in Formula (PBM-18-1A) represents N, and R^{18g} represents N. In some embodiments, R^{18f} in Formula (PBM-18-1A) represents N, and R^{18g} represents CH.

In some embodiments, R^{18f} in Formula (PBM-18-1A) represents CH, R^{18g} represents CH, and R^{18h} represents CH. In some embodiments, R^{18f} in Formula (PBM-18-1A) represents CH, R^{18g} represents N, and R^{18h} represents CH. In some embodiments, R^{18f} in Formula (PBM-18-1A) represents CH, R^{18g} represents N, and R^{18h} represents N. In some embodiments, R^{18f} in Formula (PBM-18-1A) represents CH, R^{18g} represents CH, and R^{18h} represents N. In some embodiments, R^{18f} in Formula (PBM-18-1A) represents N, R^{18g} represents N, and R^{18h} represents N. In some embodiments, R^{18f} in Formula (PBM-18-1A) represents N, R^{18g} represents CH, and R^{18h} represents N. In some embodiments, R^{18f} in Formula (PBM-18-1A) represents N, R^{18g} represents CH, and R^{18h} represents CH. In some embodiments, R^{18f} in Formula (PBM-18-1A) represents N, R^{18g} represents N, and R^{18h} represents CH.

In some embodiments, PBM represents a structure of Formula (PBM-18-1) or Formula (PBM-18-2):

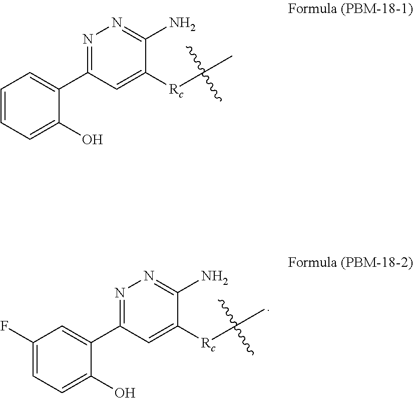

In some embodiments, PBM represents a small molecule ligand of BET.

In some embodiments, PBM represents the structure of Formula (PBM-26):

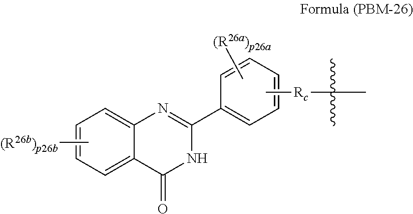

wherein (R^{26a})ₚ₂₆ₐ in Formula (PBM-26) indicates that the benzene ring to which it is attached is optionally substituted with p26a R^{26a}, wherein each R^{26a} is the same or different and independently represents deuterium, halogen (e.g., fluorine, chlorine, bromine, or iodine), hydroxy, amino, cyano, C₁₋₆ alkoxy (e.g., C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), or halogenated C₁₋₆ alkyl (e.g., halogenated C₁₋₅ alkyl, or halogenated C₁₋₃ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—);
p26a represents an integer of 0, 1, 2, 3, or 4;
(R^{26b})_{p26b} indicates that the 4-oxo-3,4-dihydroquinazoline ring to which it is attached is substituted with p26b R^{26b}, wherein each R^{26b} is the same or different and independently represents halogen (e.g., fluorine, chlorine, bromine, or iodine), hydroxy, amino, cyano, C₁₋₆ alkoxy (e.g., C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), or halogenated C₁₋₆ alkyl (e.g., halogenated C₁₋₅ alkyl, or halogenated C₁₋₃ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), and
p26b represents an integer of 1, 2, 3, or 4.

In some embodiments, PBM represents the structure of Formula (PBM-26-1):

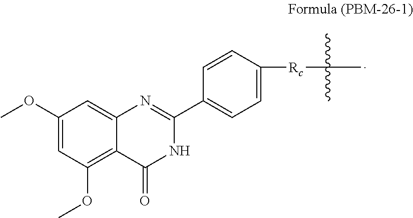

In some embodiments, PBM represents a small molecule ligand of CDK2.

In some embodiments, PBM represents the structure of Formula (PBM-29):

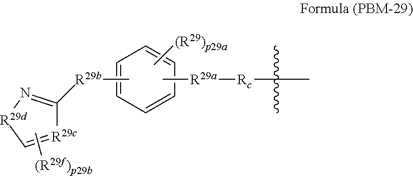

wherein R^{29a} represents S(O)₂ or a bond;
R^{29b} represents NH, NHC(O), or C(O)NH;
R^{29c} represents CH or N;
R^{29d} represents N(R^{29h}), wherein R^{29h} represents H, —C(O)-optionally substituted aryl, or —C(O)— optionally substituted heteroaryl, or
R^{29d} represents fragment

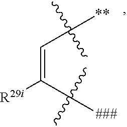

wherein symbol ** indicates the point of attachment to the adjacent nitrogen atom, symbol ###indicates the point of attachment to the adjacent carbon atom, and R²⁹ⁱ represents deuterium, hydrogen, halogen (e.g., fluorine, chlorine, bromine, or iodine), C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), halogenated C₁₋₆ alkyl (e.g., halogenated C₁₋₅ alkyl, or halogenated C₁₋₃ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), or deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.);
(R^{29e})ₚ₂ₐ indicates that the benzene ring to which it is attached is optionally substituted with p29a R^{29e}, wherein each R^{29e} is the same or different and independently represents deuterium, halogen (e.g., fluorine, chlorine, bromine, or iodine), hydroxy, amino, cyano, C₁₋₆ alkoxy, halogenated C₁₋₆ alkoxy, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), or halogenated C₁₋₆ alkyl (e.g., halogenated C₁₋₅ alkyl, or halogenated C₁₋₃ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—);
p29a represents an integer of 0, 1, 2, 3, or 4;
(R^{29f})_{p2pb} indicates that the nitrogen-containing ring to which it is attached is substituted by p29b R^{29f} substituents, wherein each R^{29f} is the same or different and independently represents deuterium, amino, halogen (e.g., fluorine, chlorine, bromine, or iodine), hydroxy, cyano, C₁₋₆ alkoxy, halogenated C₁₋₆ alkoxy, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), halogenated C₁₋₆ alkyl (e.g., halogenated C₁₋₅ alkyl, or halogenated C₁₋₃ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), —NH-optionally substituted aryl, or—NH-optionally substituted heteroaryl, and
p29b represents an integer of 1 or 2.

In some embodiments, PBM represents the structure of Formula (PBM-29-A):

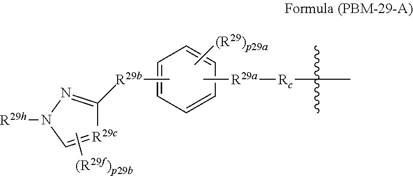

wherein R^{29a} represents S(O)₂ or a bond;
R^{29b} represents NH, NHC(O), or C(O)NH;
R^{29c} represents CH or N;
R^{29h} represents H, —C(O)-optionally substituted aryl, or —C(O)-optionally substituted heteroaryl, or
(R^{29e})ₚ₂₉ₐ indicates that the benzene ring to which it is attached is optionally substituted with p29a R^{29e} wherein each R^{29e} is the same or different and independently represents deuterium, halogen (e.g., fluorine, chlorine, bromine, or iodine), hydroxy, amino, cyano, C₁₋₆ alkoxy, halogenated C₁₋₆ alkoxy, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), or halogenated C₁₋₆ alkyl (e.g., halogenated C₁₋₅ alkyl, or halogenated C₁₋₃ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—);
p29a represents an integer of 0, 1, 2, 3, or 4;
(R^{29f})_{p29b} indicates that the nitrogen-containing ring to which it is attached is substituted by p29b R^{29f} substituents, wherein each R^{29f} is the same or different and independently represents deuterium, amino, halogen (e.g., fluorine, chlorine, bromine, or iodine), hydroxy, cyano, C₁₋₆ alkoxy, halogenated C₁₋₆ alkoxy, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), halogenated C₁₋₆ alkyl (e.g., halogenated C₁₋₅ alkyl, or halogenated C₁₋₃ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), —NH-optionally substituted aryl, or—NH-optionally substituted heteroaryl, and
p29b represents an integer of 1 or 2.

In some embodiments, PBM represents the structure of Formula (PBM-29-B):

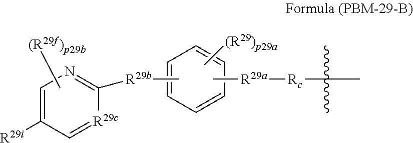

wherein R^{29a} represents S(O)₂ or a bond;
R^{29b} represents NH, NHC(O), or C(O)NH;
R^{29c} represents CH or N;
R²⁹ⁱ represents deuterium, hydrogen, halogen (e.g., fluorine, chlorine, bromine, or iodine), C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), halogenated C₁₋₆ alkyl (e.g., halogenated C₁₋₅ alkyl, or halogenated C₁₋₃ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), or deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.);
(R^{29e})ₚ₂₉ₐ indicates that the benzene ring to which it is attached is optionally substituted with p29a R^{29e}, wherein each R^{29e} is the same or different and independently represents deuterium, halogen (e.g., fluorine, chlorine, bromine, or iodine), hydroxy, amino, cyano, C₁₋₆ alkoxy, halogenated C₁₋₆ alkoxy, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), or halogenated C₁₋₆ alkyl (e.g., halogenated C₁₋₅ alkyl, or halogenated C₁₋₃ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—);
p29a represents an integer of 0, 1, 2, 3, or 4;
(R²⁹ⁱ)_{p29b} indicates that the nitrogen-containing ring to which it is attached is substituted by p29b R^{29f} substituents, wherein each R^{29f} is the same or different and independently represents deuterium, amino, halogen (e.g., fluorine, chlorine, bromine, or iodine), hydroxy, cyano, C₁₋₆ alkoxy, halogenated C₁₋₆ alkoxy, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), halogenated C₁₋₆ alkyl (e.g., halogenated C₁₋₅ alkyl, or halogenated C₁₋₃ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), —NH-optionally substituted aryl, or—NH-optionally substituted heteroaryl, and
p29b represents an integer of 1 or 2.

In some embodiments, R^{29h} in each of Formula (PBM-29) and Formula (PBM-29-A) independently represents H.

In some embodiments, R^{29h} in each of Formula (PBM-29) and Formula (PBM-29-A) independently represents-C(O)-optionally substituted aryl. In some sub-embodiments, examples of the aryl include, but are not limited to, C₆₋₂₀ aryl, C₆₋₁₅ aryl, and C₆₋₁₀ aryl, e.g., phenyl and naphthyl. The aryl is optionally substituted with one or more (e.g., 1-6, such as 1, 2, 3, 4, 5, or 6) substituents independently selected from the group consisting of deuterium, halogen (e.g., fluorine, chlorine, bromine, or iodine), hydroxy, cyano, amino, nitro, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), C₁₋₆ alkoxy (e.g., C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), C(O)NH₂, and any combination thereof.

In some embodiments, R^{29h} in each of Formula (PBM-29) and Formula (PBM-29-A) independently represents-C(O)-optionally substituted heteroaryl. In some sub-embodiments, the heteroaryl comprises 5-to 20-membered heteroaryl (e.g., 5-to 15-membered heteroaryl), e.g., 5-to 20-membered heteroaryl containing one or two 5-to 7-membered rings and 1 to 4 heteroatoms selected from N, O, and S. Examples of the heteroaryl include, but are not limited to, furanyl, oxazolyl, isoxazolyl, oxadiazolyl, thienyl, thiazolyl, isothiazolyl, thiadiazolyl, pyrrolyl, imidazolyl, pyrazolyl, triazolyl, pyridyl, pyrimidinyl, pyridazinyl, pyrazinyl, indolyl, isoindolyl, benzofuranyl, isobenzofuranyl, benzothienyl, indazolyl, benzimidazolyl, benzoxazolyl, benzisoxazolyl, benzothiazolyl, benzisothiazolyl, benzotriazolyl, benzo[2,1,3]oxadiazolyl, benzo[2,1,3]thiadiazolyl, benzo[1,2,3]thiadiazolyl, quinolinyl, isoquinolinyl, naphthyridinyl, cinnolinyl, quinazolinyl, quinoxalinyl, phthalazinyl, pyrazolo[1,5-a]pyridyl, pyrazolo[1,5-a]pyrimidinyl, imidazo[1,2-alpyridyl, 1H-pyrrolo[3,2-b]pyridyl, 1H-pyrrolo[2,3-b]pyridyl, pyrrolo[2,1-b]thiazolyl, imidazo[2,1-b]thiazolyl, imidazo[1,2-b]pyridazinyl, 6,7-dihydro-5H-pyrrolo[1,2-c]imidazolyl, and 7H-pyrrolo[2,3-d]pyrimidinyl. The heteroaryl group is optionally substituted with one or more (e.g., 1-6, such as 1, 2, 3, 4, 5, or 6) substituents independently selected from the group consisting of deuterium, halogen (e.g., fluorine, chlorine, bromine, or iodine), hydroxy, cyano, amino, nitro, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), C₁₋₆ alkoxy (e.g., C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), and any combination thereof.

In some embodiments, (R^{29f})_{p29b} in each of Formula (PBM-29), Formula (PBM-29-A), and Formula (PBM-29-B) independently indicates that the nitrogen-containing ring to which it is attached is substituted by p29b R^{29f} substituents, wherein each R^{29f} is the same or different and independently represents halogen (e.g., fluorine, chlorine, bromine, or iodine), amino, —NH-optionally substituted aryl, or—NH-optionally substituted heteroaryl, and p29b represents an integer of 1 or 2. In some sub-embodiments, examples of the aryl include, but are not limited to, C₆₋₂₀ aryl, C₆₋₁₅ aryl, and C₆₋₁₀ aryl, e.g., phenyl and naphthyl. The aryl is optionally substituted with one or more (e.g., 1-6, such as 1, 2, 3, 4, 5, or 6) substituents independently selected from the group consisting of deuterium, halogen (e.g., fluorine, chlorine, bromine, or iodine), hydroxy, cyano, amino, nitro, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), C₁₋₆ alkoxy (e.g., C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), C(O)NH₂, and any combination thereof. In some sub-embodiments, the heteroaryl comprises 5-to 20-membered heteroaryl (e.g., 5-to 15-membered heteroaryl), e.g., 5-to 20-membered heteroaryl containing one or two 5-to 7-membered rings and 1 to 4 heteroatoms selected from N, O, and S. Examples of heteroaryl include, but are not limited to, furanyl, oxazolyl, isoxazolyl, oxadiazolyl, thienyl, thiazolyl, isothiazolyl, thiadiazolyl, pyrrolyl, imidazolyl, pyrazolyl, triazolyl, pyridyl, pyrimidinyl, pyridazinyl, pyrazinyl, indolyl, isoindolyl, benzofuranyl, isobenzofuranyl, benzothienyl, indazolyl, benzimidazolyl, benzoxazolyl, benzisoxazolyl, benzothiazolyl, benzisothiazolyl, benzotriazolyl, benzo[2,1,3]oxadiazolyl, benzo[2,1,3]thiadiazolyl, benzo[1,2,3]thiadiazolyl, quinolinyl, isoquinolinyl, naphthyridinyl, cinnolinyl, quinazolinyl, quinoxalinyl, phthalazinyl, pyrazolo[1,5-a]pyridyl, pyrazolo[1,5-a]pyrimidinyl, imidazo[1,2-a]pyridyl, 1H-pyrrolo[3,2-b]pyridyl, 1H-pyrrolo[2,3-b]pyridyl, pyrrolo[2,1-b]thiazolyl, imidazo[2,1-b]thiazolyl, imidazo[1,2-b]pyridazinyl, 6,7-dihydro-5H-pyrrolo[1,2-c]imidazolyl, and 7H-pyrrolo[2,3-d]pyrimidinyl. The heteroaryl group is optionally substituted with one or more (e.g., 1-6, such as 1, 2, 3, 4, 5, or 6) substituents independently selected from the group consisting of deuterium, halogen (e.g., fluorine, chlorine, bromine, or iodine), hydroxy, cyano, amino, nitro, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), C₁₋₆ alkoxy (e.g., C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), and any combination thereof.

In some embodiments, PBM represents the structure of Formula (PBM-29-1), Formula (PBM-29-2), Formula (PBM-29-3), or Formula (PBM-29-4):

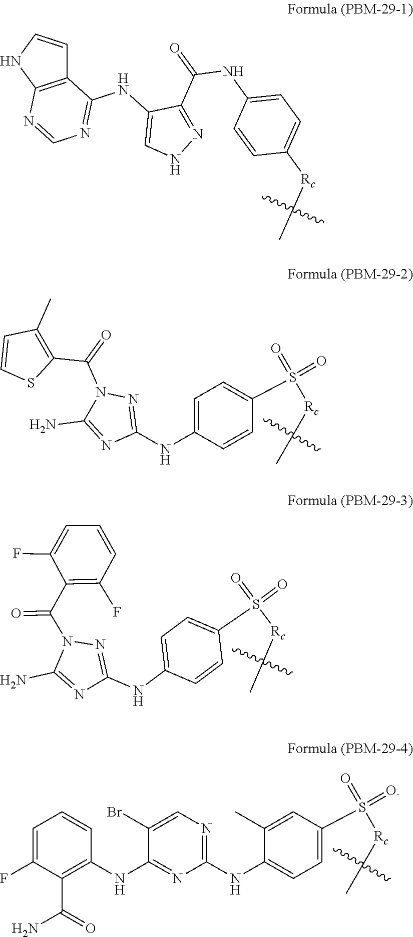

In some embodiments, PBM represents a small molecule ligand of Tau.

In some embodiments, PBM represents the structure of Formula (PBM-30):

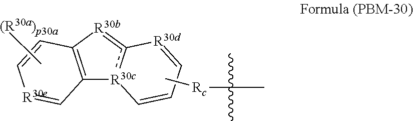

wherein (R^{30a})ₚ₃₀ₐ in Formula (PBM-30) indicates that that the fused tricyclic ring to which it is attached is optionally substituted by p30a R^{30a} substituents, wherein each R^{30a} is the same or different and independently represents deuterium, halogen (e.g., fluorine, chlorine, bromine, or iodine), hydroxy, amino, cyano, C₁₋₆ alkoxy (e.g., C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), or halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—);
p30a represents an integer of 0, 1, 2, 3, 4, 5, or 6;
[symbol] in Formula (PBM-30) represents a single bond or a double bond;
fragment

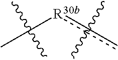

of the fused tricyclic ring in Formula (PBM-30) represents

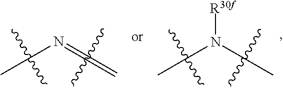

wherein R^{30f} represents hydrogen, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), or halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—);
wherein when the fragment

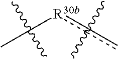

represents

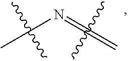 [SMILES: *N=*]

fragment

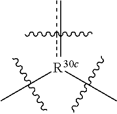

represents

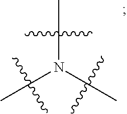 [SMILES: *N(*)*]

when the fragment

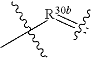

represents

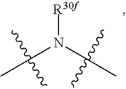

fragment

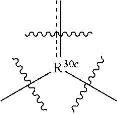

represents

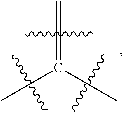 [SMILES: *C(*)=*]

and
R^{30d} and R^{30e} are the same or different and independently represent N or CH.

In some embodiments, PBM represents the structure of Formula (PBM-30-A):

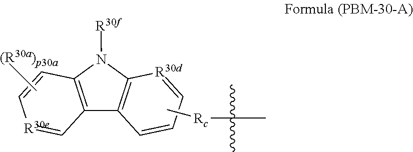

wherein (R^{30a})ₚ₃₀ₐ in Formula (PBM-30-A) indicates that that the fused tricyclic ring to which it is attached is optionally substituted by p30a R^{30a} substituents, wherein each R^{30a} is the same or different and independently represents deuterium, halogen (e.g., fluorine, chlorine, bromine, or iodine), hydroxy, amino, cyano, C₁₋₆ alkoxy (e.g., C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), or halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—);
p30a represents an integer of 0, 1, 2, 3, 4, 5, or 6;
R^{30f} represents hydrogen, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), or halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), and
R^{30d} and R^{30e} are the same or different and independently represent N, or CH.

In some embodiments, PBM represents the structure of Formula (PBM-30-B):

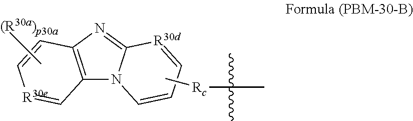

wherein (R^{30a})ₚ₃₀ₐ in Formula (PBM-30-B) indicates that that the fused tricyclic ring to which it is attached is optionally substituted by p30a R^{30a} substituents, wherein each R^{30a} is the same or different and independently represents deuterium, halogen (e.g., fluorine, chlorine, bromine, or iodine), hydroxy, amino, cyano, C₁₋₆ alkoxy (e.g., C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), or halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—);
p30a represents an integer of 0, 1, 2, 3, 4, 5, or 6, and
R^{30d} and R^{30e} are the same or different and independently represent N or CH.

In some embodiments, PBM represents the structure of Formula (PBM-30-1), Formula (PBM-30-2), Formula (PBM-30-3), Formula (PBM-30-4), or Formula (PBM-30-5):

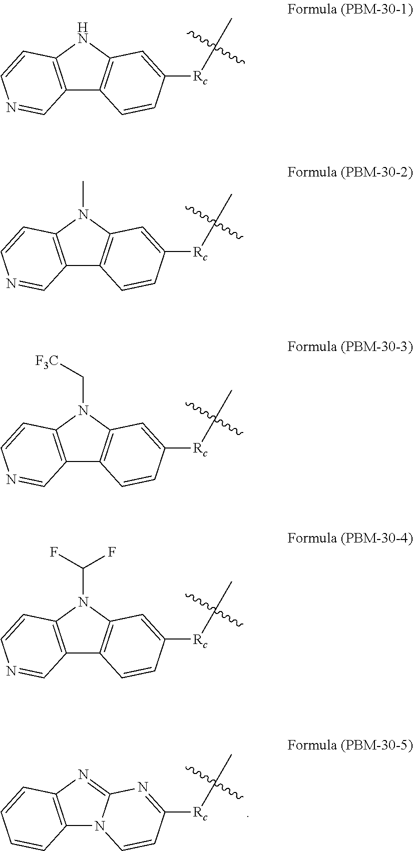

In some embodiments, PBM represents the structure of Formula (PBM-31):

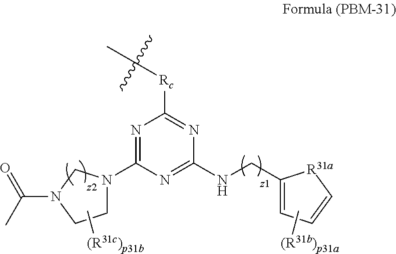

wherein R^{31a} in Formula (PBM-31) represents O, S, or NH;
(R^{31b})ₚ₃₁ₐ indicates that the 5-membered ring containing R^{31a} to which it is attached is optionally substituted with p31a R^{31b}, wherein each R^{31b} is the same or different and independently represents deuterium, halogen (e.g., fluorine, chlorine, bromine, or iodine), hydroxy, amino, cyano, C₁₋₆ alkoxy (e.g., C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), or halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—);
p31a represents an integer of 0, 1, 2, or 3;
(R^{31c})_{p31b} indicates that the diazaheterocyclyl to which it is attached is optionally substituted by p31b R^{31c} substituents, wherein each R^{31c} is the same or different and independently represents deuterium, halogen (e.g., fluorine, chlorine, bromine, or iodine), hydroxy, amino, cyano, C₁₋₆ alkoxy (e.g., C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), or halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—);
p31b represents an integer of 0, 1, 2, 3, 4, 5, 6, 7, 8, 9, or 10;
Z1 represents an integer of 1, 2, or 3, and
Z2 represents an integer of 1, 2, 3, 4, 5, or 6.

In some embodiments, PBM represents the structure of Formula (PBM-31-1):

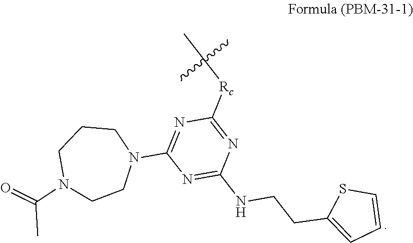

In some embodiments, PBM represents a small molecule ligand of PARP.

In some embodiments, PBM represents the structure of Formula (PBM-33):

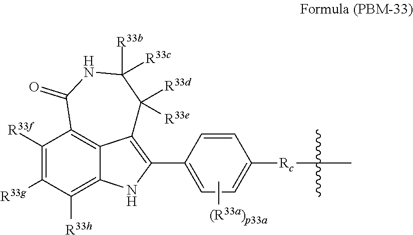

wherein (R^{33a})ₚ₃₃ₐ in Formula (PBM-33) indicates that the benzene ring to which it is attached is optionally substituted with p33a R^{33a} substituents, wherein each R^{33a} is the same or different and independently represents deuterium, halogen (e.g., fluorine, chlorine, bromine, or iodine), hydroxy, amino, cyano, C₁₋₆ alkoxy (e.g., C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), or halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—);
p33a represents an integer of 0, 1, 2, 3, 4, or 5, and
R^{33b}, R^{33c}, R^{33d}, R^{33e}, R^{33f}, R^{33g}, and R^{33h} are the same or different and independently represent hydrogen, deuterium, halogen (e.g., fluorine, chlorine, bromine, or iodine), hydroxy, amino, cyano, C₁₋₆ alkoxy (e.g., C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), or halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—).

In some embodiments, PBM represents the structure of Formula (PBM-33-1):

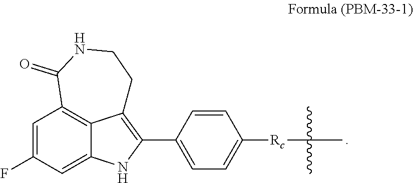

In the present disclosure, the PBM is connected to the LIN via R_{c} in its general formula.

In some embodiments, R_{c} in each of the general formulae of PBM represents a bond, —O—, —NHC(O)—*, —C(O)NH—*, —C(O)O—*, —OC(O)—*, or —NH—.

In some embodiments, R_{c} in each of the general formulae of PBM represents a structure of the following Formulae:

—N(R_{d})—, —N(R_{d})—R_{f}—N(R_{f})—*, —R_{f}—C(O)NH—*, —R_{f}—NHC(O)—*, —R_{f}—C(O)O—*, —R_{f}—OC(O)—*, —R_{f}—, —R_{f}—N(R_{d})—*, —O—R_{f}—N(R_{d})—*,

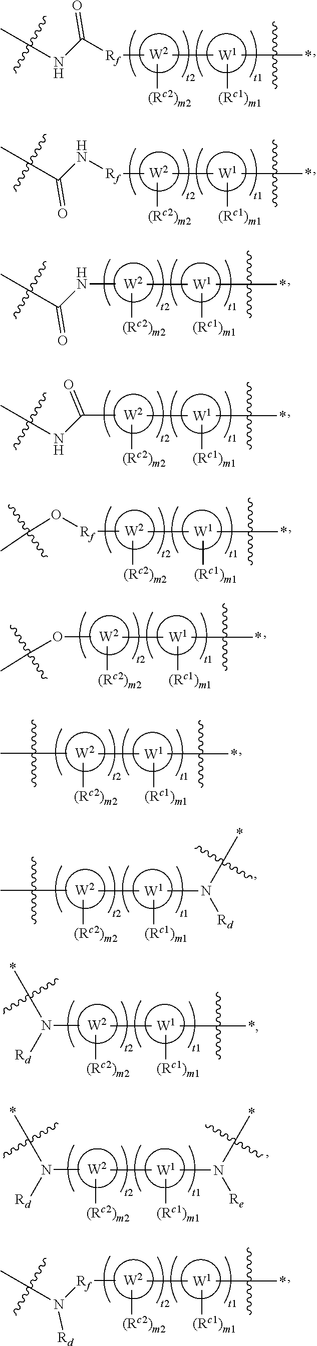

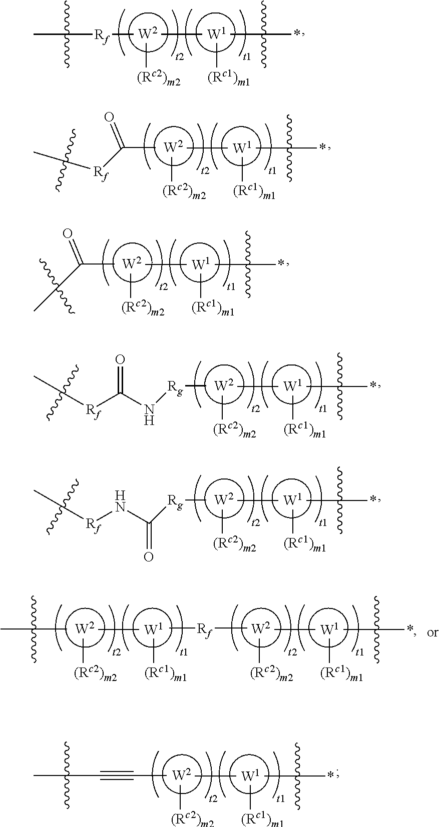

wherein each ring W¹ is the same or different and each independently represents 4-to 20-membered nitrogen-containing heterocyclylene, t1 represents an integer of 1 or 2, and (R^{c1})ₘ₁ indicates that each ring W¹ is independently optionally substituted with m1 Rel groups, wherein each Rel is independently deuterium, C₁₋₆ alkyl, deuterated C₁₋₆ alkyl, halogenated C₁₋₆ alkyl, C₁₋₆ alkoxy, halogenated C₁₋₆ alkoxy, deuterated C₁₋₆ alkoxy, halogen, amino, hydroxy, mercapto, cyano, carboxyl, C₂₋₆ alkynyl, C₂₋₆ alkenyl, optionally deuterated C₃₋₆ cycloalkyl, or R^{c4}—R_{c3}—, wherein R_{c3} is optionally substituted C₁₋₆ alkylene or optionally substituted C₂₋₆ alkenylene, and R^{c4} represents halogen, R_{c5}R^{c6}N—, hydroxy, carboxyl, ethynyl, or vinyl, wherein R^{c5} and R_{c6} are the same or different and independently represent hydrogen or C₁₋₃ alkyl, and m1 represents an integer of 0-20;
each ring W² is the same or different and each independently represents 4-to 20-membered nitrogen-containing heterocyclylene, C₃₋₂₀ cycloalkylene, C₅₋₂₀ arylene, or 5-to 20-membered heteroarylene, t2 represents an integer of 0 or 1, and (R_{c2})ₘ₂ indicates that each ring W² is independently optionally substituted with m2 R_{c2} groups, wherein each R_{c2} is independently deuterium, C₁₋₆ alkyl, deuterated C₁₋₆ alkyl, halogenated C₁₋₆ alkyl, C₁₋₆ alkoxy, halogenated C₁₋₆ alkoxy, deuterated C₁₋₆ alkoxy, halogen, amino, hydroxy, mercapto, cyano, carboxyl, C₂₋₆ alkynyl, C₂₋₆ alkenyl, optionally deuterated C₃₋₆ cycloalkyl, or R_{c8}—R_{c7}—, wherein R_{c7} is optionally substituted C₁₋₆ alkylene or optionally substituted C₂₋₆alkenylene, and R_{c8} represents halogen, R_{c9}R^{c10}N—, hydroxy, carboxyl, ethynyl, or vinyl, wherein R_{c9} and R^{c10} are the same or different and independently represent hydrogen or C₁₋₃ alkyl, and m2 represents an integer of 0-20; and
R_{d} and Rₑ each independently represent hydrogen or C₁₋₆ alkyl; R_{f} and R_{g} each independently represent optionally substituted C₁₋₆ alkylene or optionally substituted C₂₋₆ alkenylene;
the C₁₋₆ alkylene and C₂₋₆ alkenylene are each independently optionally substituted with one or more substituents selected from the group consisting of deuterium, halogen, hydroxy, mercapto, cyano, amino, nitro, C₁₋₆ alkyl, halogenated C₁₋₆ alkyl, deuterated C₁₋₆ alkyl, C₁₋₆ alkoxy, or halogenated C₁₋₆ alkoxy; and symbol * indicates the point of attachment to LIN.

In some embodiments, R^{a} and Rₑ each independently represent hydrogen or C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl).

In some embodiments, R^{f} and R_{g} each independently represent C₁₋₆ alkylene and C₂₋₆ alkenylene. The C₁₋₆ alkylene and C₂₋₆ alkenylene are optionally substituted with one or more (e.g., 1-5, such as 1, 2, 3, 4, or 5) substituents selected from the group consisting of deuterium, halogen, hydroxy, mercapto, cyano, amino, nitro, C₁₋₆ alkyl, halogenated C₁₋₃ alkyl, deuterated C₁₋₆ alkyl, C₁₋₆ alkoxy, or halogenated C₁₋₆ alkoxy, and any combination thereof.

In some embodiments, t1 represents an integer of 1.

In some embodiments, t1 represents an integer of 2.

In some embodiments, each ring W¹ is the same or different and each independently represents 4-to 20-membered (e.g., 4-to 15-membered, 4-to 12-membered, 4-to 11-membered, 4-to 10-membered, 4-to 9-membered, 4-to 8-membered, 4-to 7-membered, 4-to 6-membered, 5-to 15-membered, or 5-to 9-membered) nitrogen-containing heterocyclylene, e.g., 4-to 20-membered (e.g., 4-to 15-membered, 4-to 12-membered, 4-to 11-membered, 4-to 10-membered, 4-to 9-membered, 4-to 8-membered, 4-to 7-membered, 4-to 6-membered, 5-to 15-membered, or 5-to 9-membered) heterocyclylene containing one nitrogen atom and optionally containing a heteroatom(s) selected from nitrogen, oxygen, and sulfur. Examples of the nitrogen-containing heterocyclylene include, but are not limited to, e.g., piperidinylene, piperazinylene, morpholinylene, azetidinylene, pyrrolidinylene, imidazolidylene, pyrazolidylene, oxazolidinylene, thiazolidinylene, thiomorpholinylene, azepanylene, diazacycloheptanylene, azacyclooctylene, diazacyclooctylene, azabicyclo[3.1.1]heptanylene, azabicyclo[2.2.1]heptanylene, azabicyclo[3.2.1]octanylene, azabicyclo[2.2.2]octanylene, diazabicyclo[3.1.1]heptanylene, diazabicyclo[2.2.1]heptanylene, diazabicyclo[3.2.1]octanylene, diazabicyclo[2.2.2]octanylene, 3-azaspiro[5.5]undecanylene, 8-azaspiro[4.5]decanylene, 2-oxa-8-azaspiro[4.5]decanylene, and 3-methyl-2-oxa-8-azaspiro[4.5]decanylene. Each ring W¹ is independently optionally substituted with m1 Rel groups, wherein each Rel is independently deuterium, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), C₁₋₆ alkoxy (e.g., C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), deuterated C₁₋₆ alkoxy, halogen (e.g., fluorine, chlorine, bromine, or iodine), amino, hydroxy, mercapto, cyano, carboxyl, C₂₋₆ alkynyl (e.g., C₂₋₄ alkynyl, such as ethynyl, propynyl or butynyl), C₂₋₆ alkenyl (e.g., C₂₋₄ alkenyl, such as vinyl, propenyl, or butenyl), optionally deuterated C₃₋₆ cycloalkyl (e.g., optionally perdeuterated cyclopropyl, optionally perdeuterated cyclobutyl, optionally perdeuterated cyclopentyl, or optionally perdeuterated cyclohexyl), or R^{c4}—R_{c3}—, wherein R_{c3} is optionally substituted C₁₋₆ alkylene (e.g., optionally substituted C₁₋₄ alkylene, such as optionally substituted methylene, ethylene, or propylene) or optionally substituted C₂₋₆ alkenylene (e.g., optionally substituted C₂₋₄ alkenylene, such as optionally substituted vinylene, propenylene, butenylene), and R^{c4} represents halogen (e.g., fluorine, chlorine, bromine, or iodine), R_{c5}R_{c6}N—, hydroxy, carboxyl, ethynyl, or vinyl, wherein R_{c5} and R_{c6} are the same or different and independently represent hydrogen, or C₁₋₃ alkyl (e.g., methyl, ethyl, or propyl); and the C₁₋₆ alkylene and C₂₋₆alkenylene are optionally substituted with one or more (e.g., 1-5, such as 1, 2, 3, 4, or 5) substituents selected from the group consisting of deuterium, halogen (e.g., fluorine, chlorine, bromine, or iodine), hydroxy, mercapto, cyano, amino, nitro, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), halogenated C₁₋₃ alkyl (e.g., halogenated C₁₋₂ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), C₁₋₃ alkoxy (e.g., methoxy, ethoxy, propoxy, or isopropoxy), halogenated C₁₋₃ alkoxy (e.g., halogenated C₁₋₂ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), and any combination thereof. m1 represents an integer of 0-20, e.g., an integer of 0, 1, 2, 3, 4, 5, 6, 7, 8, 9, 10, 11, 12, 13, 14, 15, or 20. The number of substituents is not theoretically limited in any way, or is automatically limited by the size of the building units.

In some embodiments, each ring W¹ is the same or different and each independently represents the following optionally substituted groups:

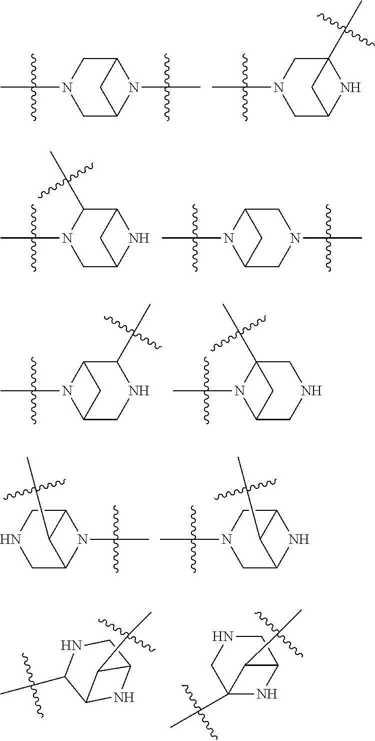

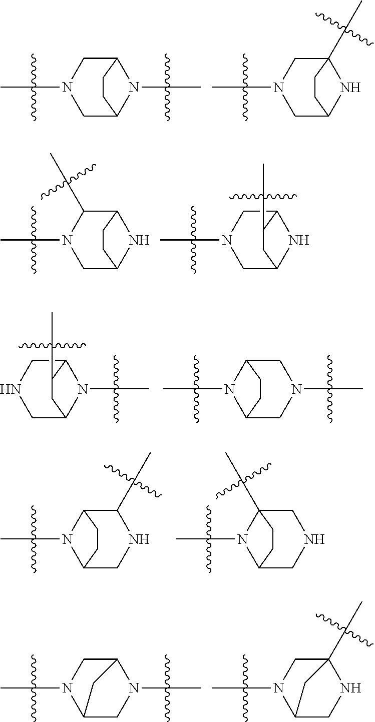

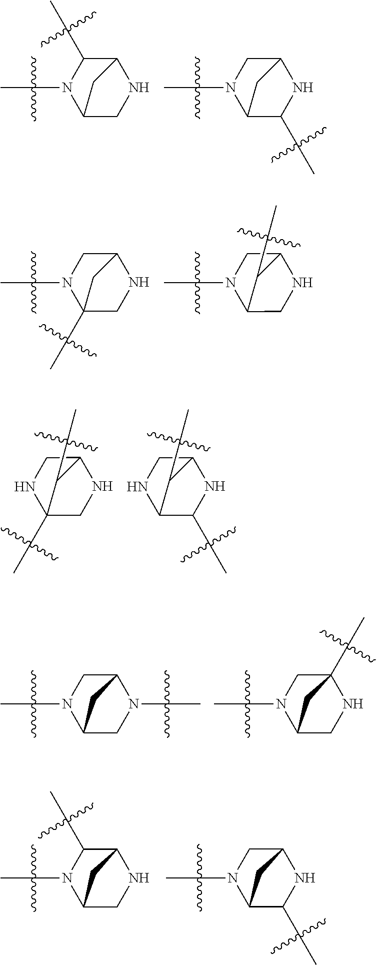

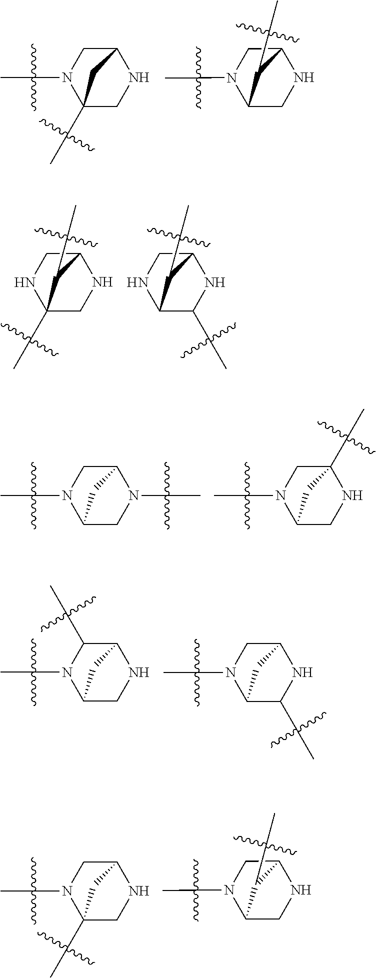

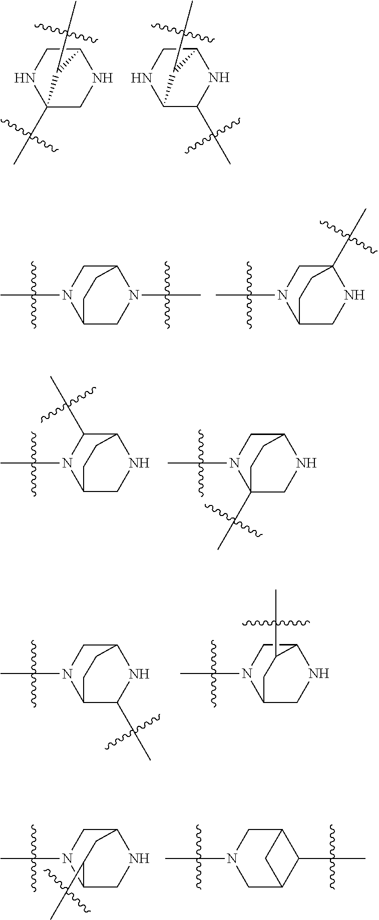

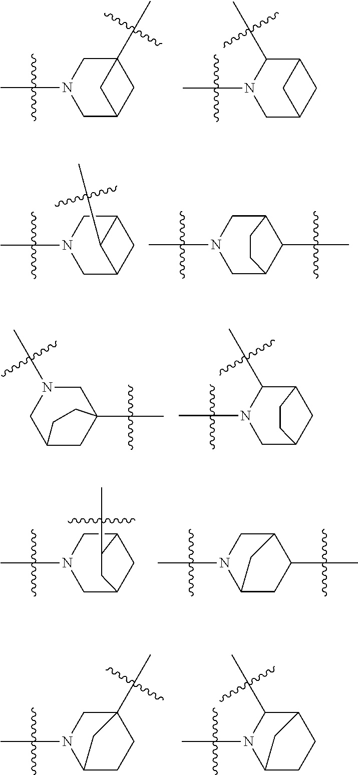

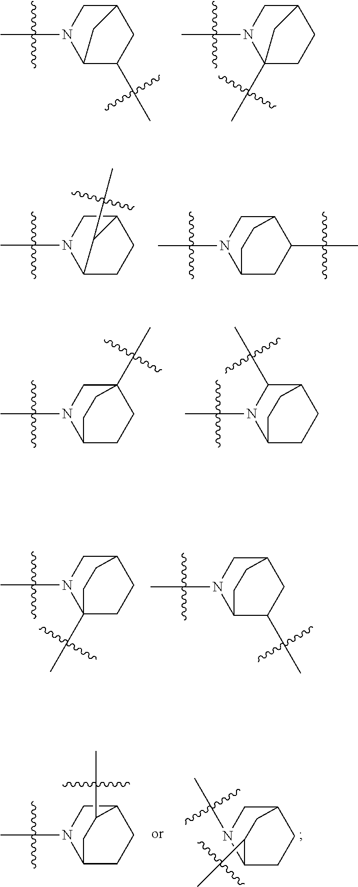

each ring W¹ is independently optionally substituted with m1 Rel groups, with each Rel being independently deuterium, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), C₁₋₆ alkoxy (e.g., C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), deuterated C₁₋₆ alkoxy, halogen (e.g., fluorine, chlorine, bromine, or iodine), amino, hydroxy, mercapto, cyano, carboxyl, C₂₋₆ alkynyl (e.g., C₂₋₄ alkynyl, such as ethynyl, propynyl or butynyl), C₂₋₆ alkenyl (e.g., C₂₋₄ alkenyl, such as vinyl, propenyl, or butenyl), optionally deuterated C₃₋₆ cycloalkyl (e.g., optionally perdeuterated cyclopropyl, optionally perdeuterated cyclobutyl, optionally perdeuterated cyclopentyl, or optionally perdeuterated cyclohexyl), or R^{c4}—R_{c3}—, wherein R_{c3} is optionally substituted C₁₋₆ alkylene (e.g., optionally substituted C₁₋₄ alkylene, such as optionally substituted methylene, ethylene, or propylene) or optionally substituted C₂₋₆ alkenylene (e.g., optionally substituted C₂₋₄alkenylene, such as optionally substituted vinylene, propenylene, butenylene), and R^{c4} represents halogen (e.g., fluorine, chlorine, bromine, or iodine), R_{c5}R_{c6}N—, hydroxy, carboxyl, ethynyl, or vinyl, wherein R_{c5} and R_{c6} are the same or different and independently represent hydrogen or C₁₋₃ alkyl (e.g., methyl, ethyl, or propyl); and the C₁₋₆ alkylene and C₂₋₆ alkenylene are optionally substituted with one or more substituents selected from the group consisting of deuterium, halogen (e.g., fluorine, chlorine, bromine, or iodine), hydroxy, mercapto, cyano, amino, nitro, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), halogenated C₁₋₃ alkyl (e.g., halogenated C₁₋₂ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), C₁₋₃ alkoxy (e.g., methoxy, ethoxy, propoxy, or isopropoxy), halogenated C₁₋₃ alkoxy (e.g., halogenated C₁₋₂ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), and any combination thereof. m1 represents an integer of 0-20, e.g., an integer of 0, 1, 2, 3, 4, 5, 6, 7, 8, 9, 10, 11, 12, 13, 14, 15, or 20. The number of substituents is not theoretically limited in any way, or is automatically limited by the size of the building units.

In some embodiments, each ring W² is the same or different and independently represents 4-to 20-membered (e.g., 4-to 15-membered, 4-to 12-membered, 4-to 11-membered, 4-to 10-membered, 4-to 9-membered, 4-to 8-membered, 4-to 7-membered, 4-to 6-membered, 5-to 15-membered, or 5-to 9-membered) nitrogen-containing heterocyclylene, C₃₋₂₀ cycloalkylene (e.g., C₃₋₁₅ cycloalkylene or C₃₋₁₁ cycloalkylene), C₅₋₂₀ arylene (e.g., C₆₋₂₀ arylene, C₅₋₁₅ arylene, or C₆₋₁₅ arylene), or 5-to 20-membered (e.g., 5-to 15-membered, 5-to 10-membered, 5-to 9-membered, 5-to 8-membered, 5-to 7-membered, or 5-to 6-membered) heteroarylene. In some embodiments, the 4-to 20-membered nitrogen-containing heterocyclylene is a 4-to 20-membered (e.g., 4-to 15-membered, 4-to 12-membered, 4-to 11-membered, 4-to 10-membered, 4-to 9-membered, 4-to 8-membered, 4-to 7-membered, 4-to 6-membered, 5-to 15-membered, or 5-to 9-membered) heterocyclylene containing one nitrogen atom and optionally containing a heteroatom(s) selected from nitrogen, oxygen, and sulfur. Examples of the 4-to 20-membered nitrogen-containing heterocyclylene include, but are not limited to, e.g., piperidinylene, piperazinylene, morpholinylene, azetidinylene, pyrrolidinylene, imidazolidylene, pyrazolidylene, oxazolidinylene, thiazolidinylene, thiomorpholinylene, azepanylene, diazacycloheptanylene, azacyclooctylene, diazacyclooctylene, azabicyclo[3.1.1]heptanylene, azabicyclo[2.2.1]heptanylene, azabicyclo[3.2.1]octanylene, azabicyclo[2.2.2]octanylene, diazabicyclo[3.1.1]heptanylene, diazabicyclo[2.2.1]heptanylene, diazabicyclo[3.2.1]octanylene, diazabicyclo[2.2.2]octanylene, 3-azaspiro[5.5]undecanylene, 8-azaspiro[4.5]decanylene, 2-oxa-8-azaspiro[4.5]decanylene, and 3-methyl-2-oxa-8-azaspiro[4.5]decanylene. In some embodiments, examples of the C₃₋₂₀ cycloalkylene include, but are not limited to, e.g., cyclopropylene, cyclobutylene, cyclopentylene, cyclopentenylene, cyclohexylene, cyclohexenylene, cycloheptylene, cyclooctylene, decalinylene, octahydropentalenylene, octahydro-1H-indenylene, 2,3-dihydro-1H-indenylene, spiro-cycloalkylene (e.g., C₅₋₁₅ spiro-cycloalkylene), adamantanylene, noradamantanylene, and norbornylene. In some embodiments, examples of the 5-to 20-membered arylene include, but are not limited to, e.g., phenylene or naphthylene. In some embodiments, the 5-to 20-membered heteroarylene is a 5-to 20-membered (e.g., 5-to 15-membered, 5-to 12-membered, 5-to 9-membered, 5-to 8-membered, 5-to 7-membered, or 5-to 6-membered) heteroarylene containing one nitrogen atom and optionally containing a heteroatom(s) selected from nitrogen, oxygen, and sulfur. In some embodiments, examples of the 5-to 20-membered heteroarylene include, but are not limited to, e.g., oxazolylene, isoxazolylene, oxadiazolylene, thienylene, thiazolylene, isothiazolylene, thiadiazolylene, pyrrolylene, imidazolylene, pyrazolylene, triazolylene, pyridylene, pyrimidinylene, pyridazinylene, pyrazinylene, indolylene, isoindolylene, benzofuranylene, isobenzofuranylene, benzothienylene, indazolylene, benzimidazolylene, benzoxazolylene, benzisoxazolylene, benzothiazolylene, benzisothiazolylene, benzotriazolylene, benzo[2,1,3]oxadiazolylene, benzo[2,1,3]thiadiazolylene, benzo[1,2,3]thiadiazolylene, quinolinylene, isoquinolinylene, naphthyridinylene, cinnolinylene, quinazolinylene, quinoxalinylene, phthalazinylene, pyrazolo[1,5-a]pyridylene, pyrazolo[1,5-alpyrimidinylene, imidazo[1,2-a]pyridylene, 1H-pyrrolo[3,2-b]pyridylene, 1H-pyrrolo[2,3-b]pyridylene, pyrrolo[2,1-b]thiazolylene, and imidazo[2,1-b]thiazolylene. Each ring W² is independently optionally substituted with m2 R_{c2} groups, with each R_{c2} being independently deuterium, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), C₁₋₆ alkoxy (e.g., C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), deuterated C₁₋₆ alkoxy, halogen (e.g., fluorine, chlorine, bromine, or iodine), amino, hydroxy, mercapto, cyano, carboxyl, C₂₋₆ alkynyl (e.g., C₂₋₄ alkynyl, such as ethynyl, propynyl or butynyl), C₂₋₆ alkenyl (e.g., C₂₋₄ alkenyl, such as vinyl, propenyl, or butenyl), optionally deuterated C₃₋₆ cycloalkyl (e.g., optionally perdeuterated cyclopropyl, optionally perdeuterated cyclobutyl, optionally perdeuterated cyclopentyl, or optionally perdeuterated cyclohexyl), or R_{c8}—R_{c7}—, wherein R_{c7} represents optionally substituted C₁₋₆ alkylene (e.g., optionally substituted C₁₋₄ alkylene, such as optionally substituted methylene, ethylene, or propylene) or optionally substituted C₂₋₆ alkenylene (e.g., optionally substituted C₂₋₄ alkenylene, such as optionally substituted vinylene, propenylene, butenylene), and R_{c8} represents halogen (e.g., fluorine, chlorine, bromine, or iodine), R_{c9}R^{c10}N—, hydroxy, carboxyl, ethynyl, or vinyl, wherein R_{c9} and R^{c10} are the same or different and independently represent hydrogen or C₁₋₃ alkyl (e.g., methyl, ethyl, or propyl); and the C₁₋₆ alkylene and C₂₋₆ alkenylene are each independently optionally substituted with one or more (e.g., 1-5, such as 1, 2, 3, 4, or 5) substituents selected from the group consisting of deuterium, halogen, hydroxy, mercapto, cyano, amino, nitro, C₁₋₆ alkyl, halogenated C₁₋₆ alkyl, deuterated C₁₋₆ alkyl, C₁₋₆ alkoxy, and halogenated C₁₋₆ alkoxy. m2 represents an integer of 0-20, e.g., an integer of 0, 1, 2, 3, 4, 5, 6, 7, 8, 9, 10, 11, 12, 13, 14, 15, or 20. The number of substituents is not theoretically limited in any way, or is automatically limited by the size of the building units.

In some embodiments, each ring W² is the same or different and independently represents:

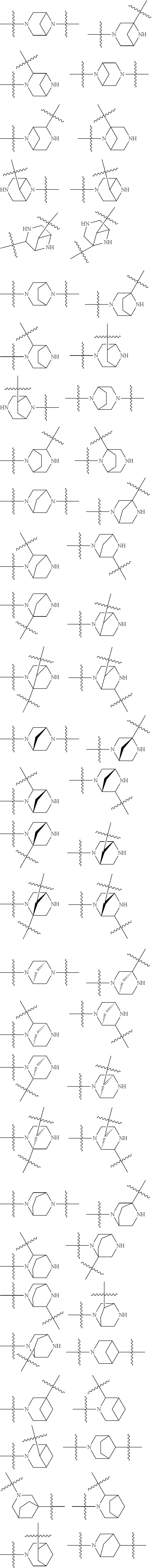

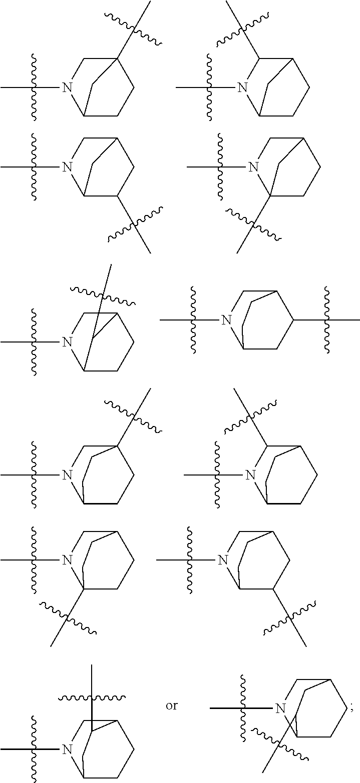

each ring W² is independently optionally substituted with m2 R_{c2} groups, with each R_{c2} being independently deuterium, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), C₁₋₆ alkoxy (e.g., C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), deuterated C₁₋₆ alkoxy, halogen (e.g., fluorine, chlorine, bromine, or iodine), amino, hydroxy, mercapto, cyano, carboxyl, C₂₋₆ alkynyl (e.g., C₂₋₄ alkynyl, such as ethynyl, propynyl or butynyl), C₂₋₆ alkenyl (e.g., C₂₋₄ alkenyl, such as vinyl, propenyl, or butenyl), optionally deuterated C₃₋₆ cycloalkyl (e.g., optionally perdeuterated cyclopropyl, optionally perdeuterated cyclobutyl, optionally perdeuterated cyclopentyl, or optionally perdeuterated cyclohexyl), or R_{c8}—R_{c7}—, wherein R_{c7} represents optionally substituted C₁₋₆ alkylene (e.g., optionally substituted C₁₋₄ alkylene, such as optionally substituted methylene, ethylene, or propylene) or optionally substituted C₂₋₆ alkenylene (e.g., optionally substituted C₂₋₄ alkenylene, such as optionally substituted vinylene, propenylene, butenylene), and R_{c8} represents halogen (e.g., fluorine, chlorine, bromine, or iodine), R_{c9}R^{c10}N—, hydroxy, carboxyl, ethynyl, or vinyl, wherein R_{c9} and R^{c10} are the same or different and independently represent hydrogen or C₁₋₃ alkyl (e.g., methyl, ethyl, or propyl); and the C₁₋₆ alkylene and C₂₋₆ alkenylene are each independently optionally substituted with one or more (e.g., 1-5, such as 1, 2, 3, 4, or 5) substituents selected from the group consisting of deuterium, halogen, hydroxy, mercapto, cyano, amino, nitro, C₁₋₆ alkyl, halogenated C₁₋₆ alkyl, deuterated C₁₋₆ alkyl, C₁₋₆ alkoxy, or halogenated C₁₋₆ alkoxy. m2 represents an integer of 0-20, e.g., an integer of 0, 1, 2, 3, 4, 5, 6, 7, 8, 9, 10, 11, 12, 13, 14, 15, or 20. The number of substituents is not theoretically limited in any way, or is automatically limited by the size of the building units.

In some embodiments, t2 represents an integer of 0.

In some embodiments, t2 represents an integer of 1.

In some embodiments, the moiety

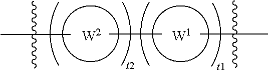

in each of the general formulae of R_{c} represents the following groups:

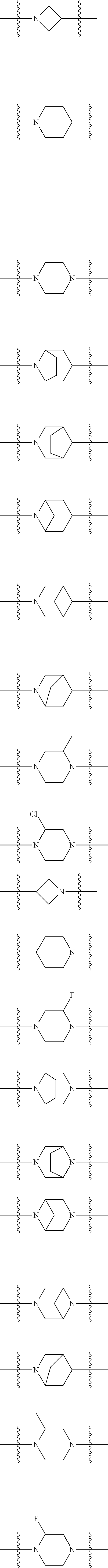

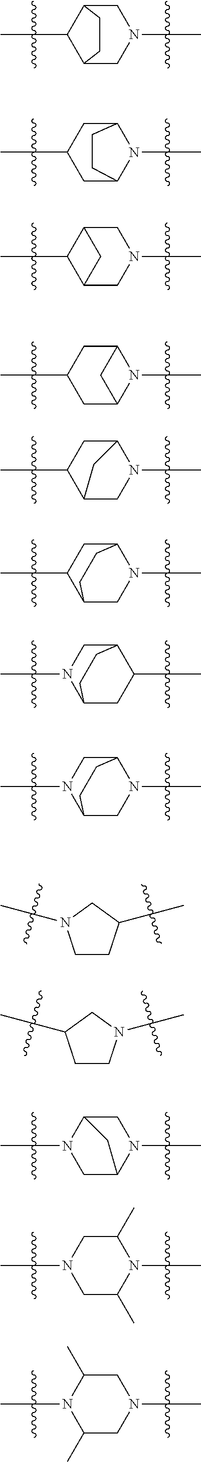

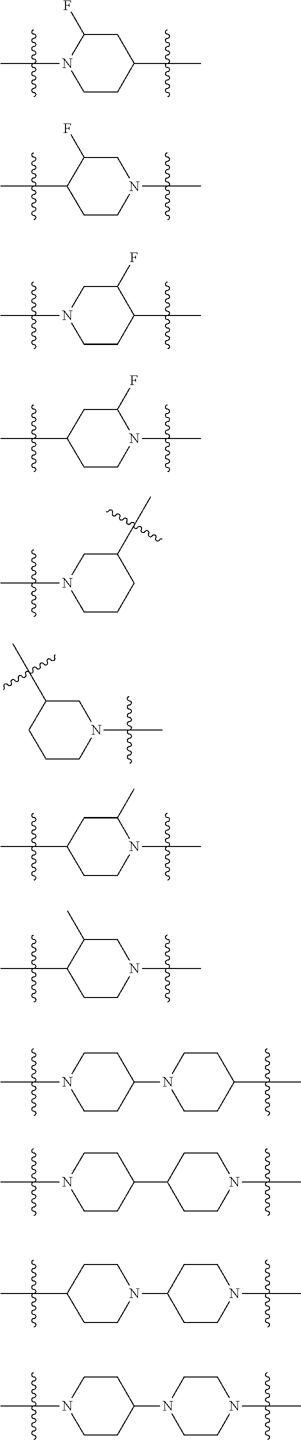

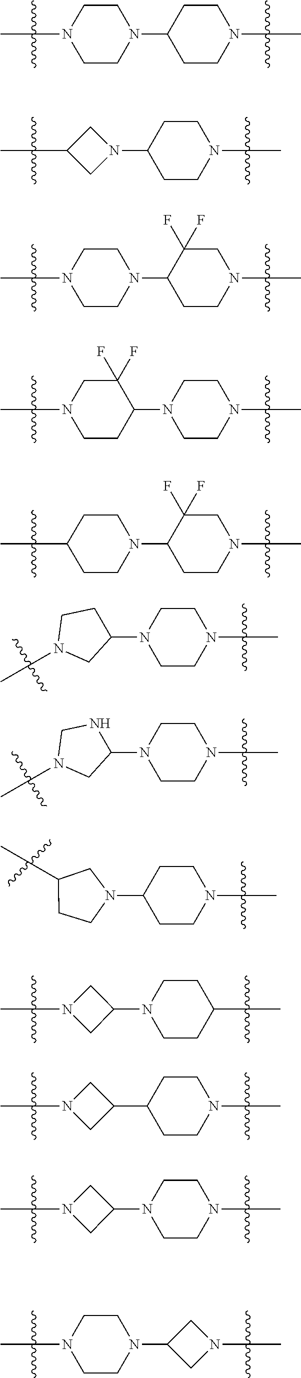

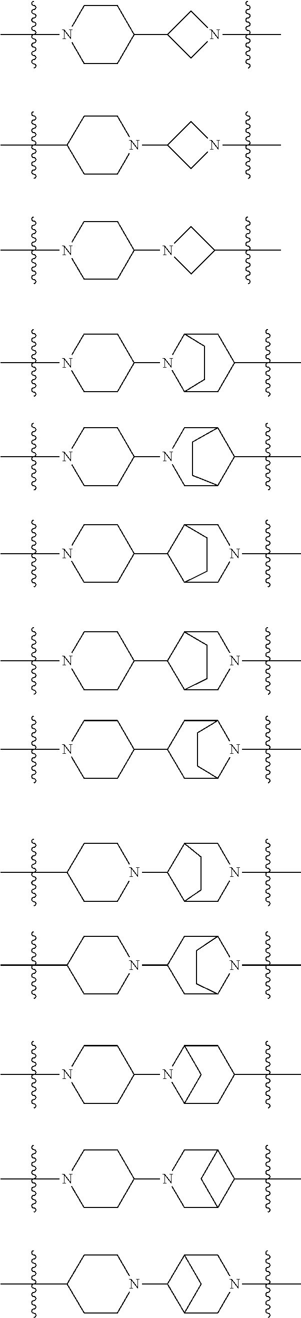

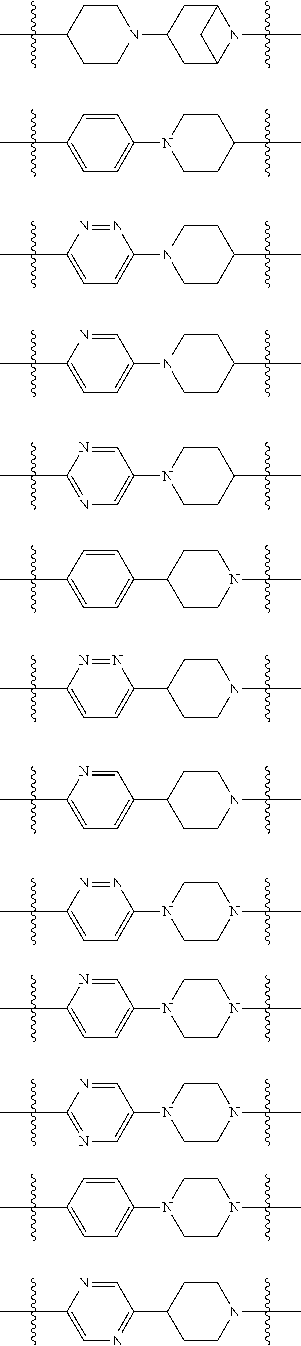

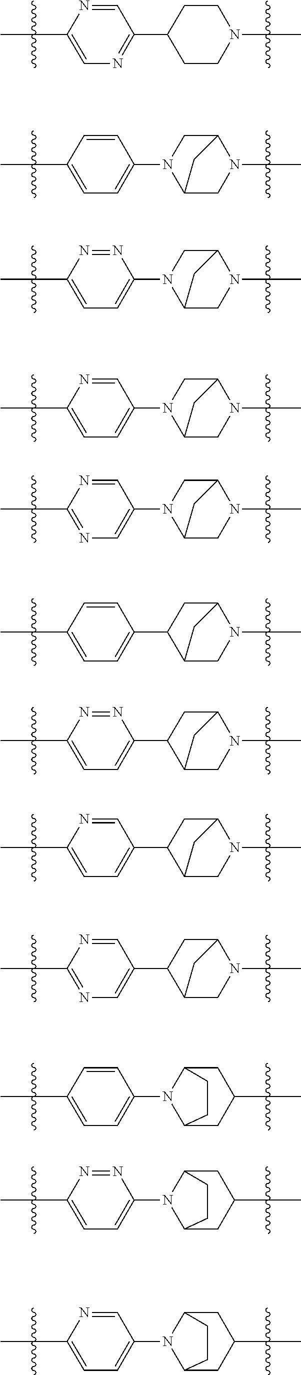

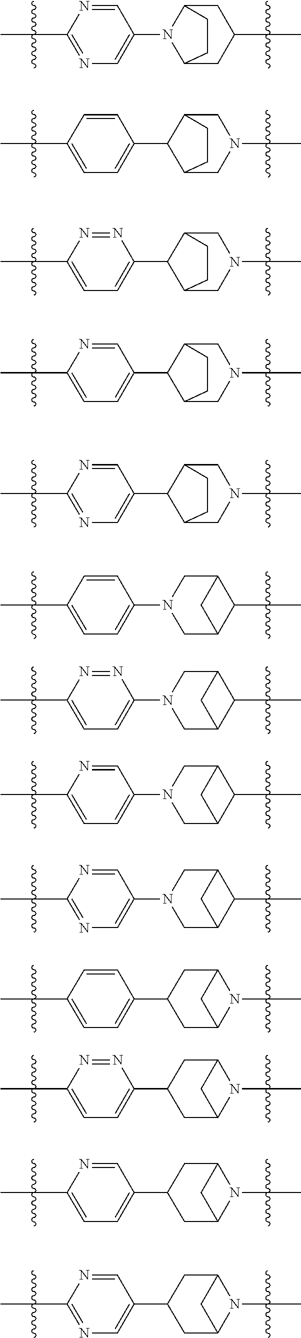

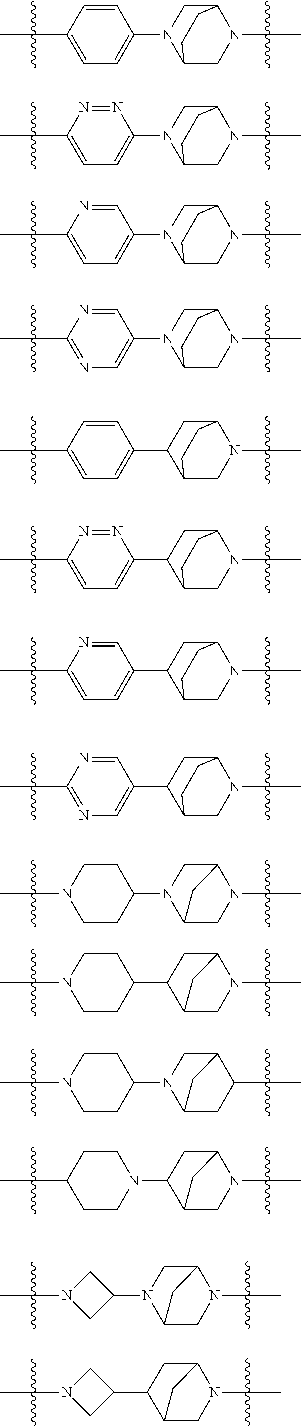

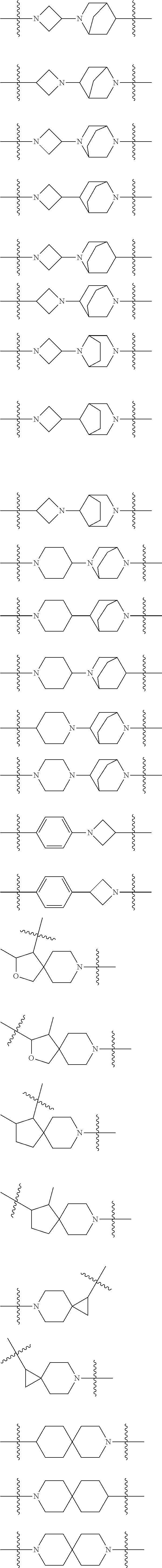

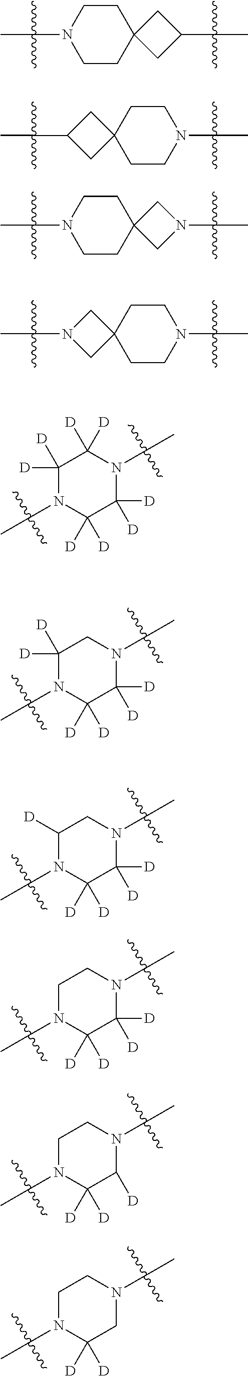

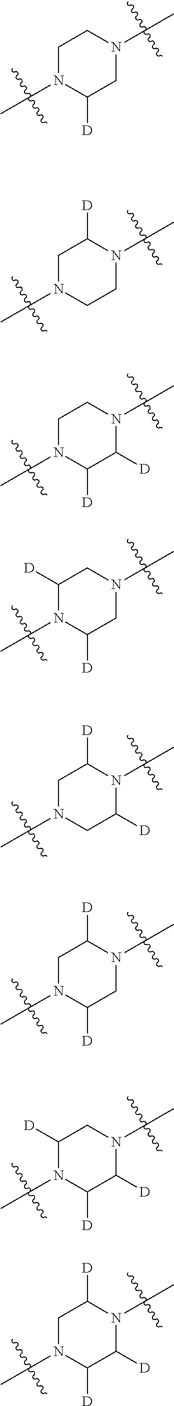

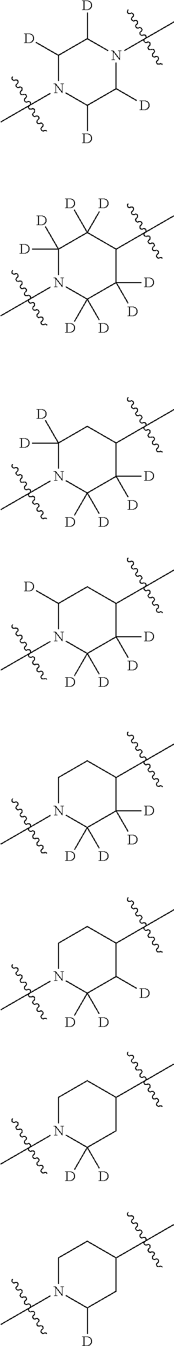

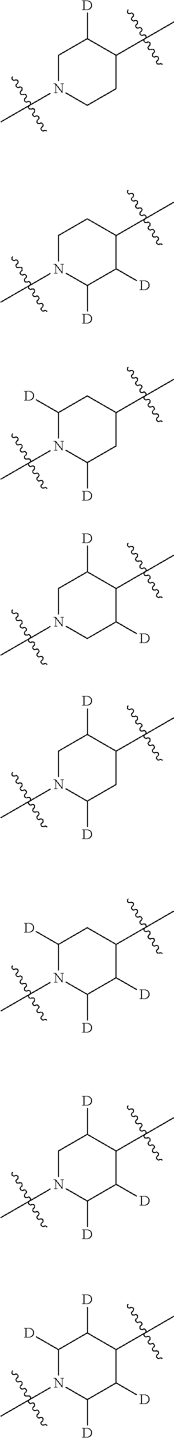

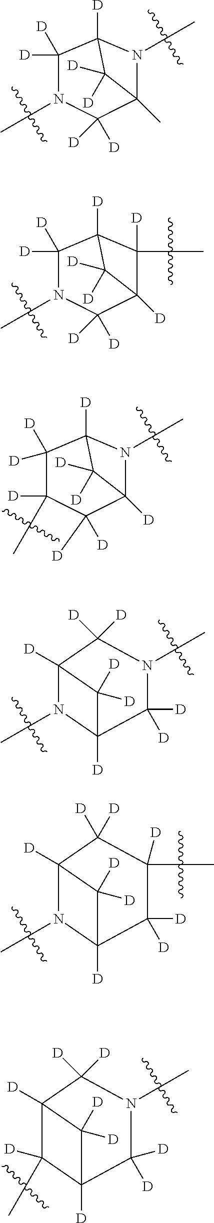

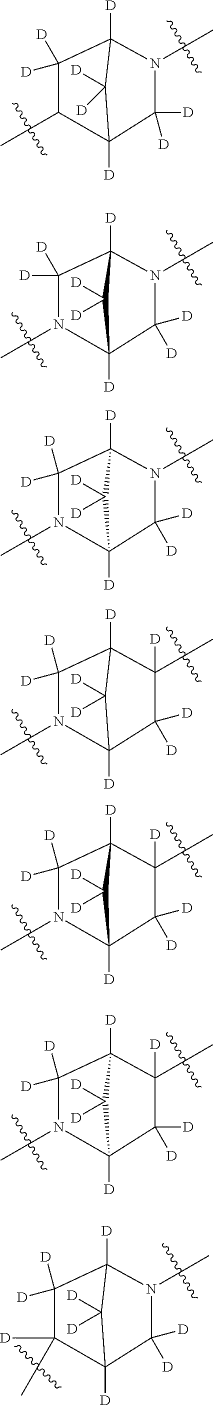

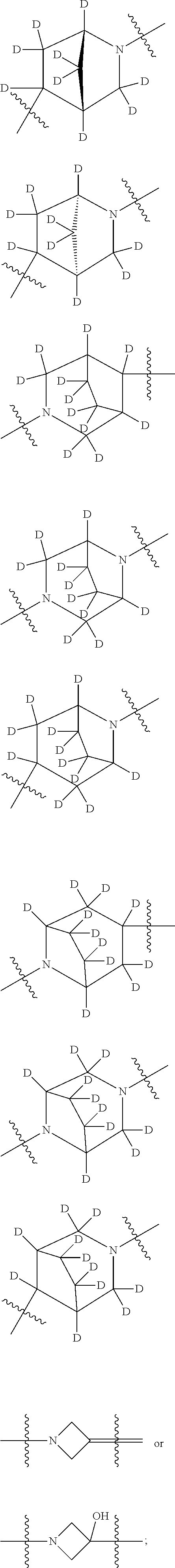

wherein the groups are each independently optionally substituted with one or more (e.g., 1-5, such as 1, 2, 3, 4, or 5) substituents selected from the group consisting of deuterium, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), C₁₋₆ alkoxy (e.g., C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), deuterated C₁₋₆ alkoxy (e.g., perdeuterated C₁₋₄ alkoxy, such as CD₃-O—, CD₃CD₂—O—, or CD₃CD₂CD₂—O—), halogen (e.g., fluorine, chlorine, bromine, or iodine), amino, hydroxy, mercapto, cyano, carboxyl, C₂₋₆ alkynyl (e.g., C₂₋₄ alkynyl, such as ethynyl, propynyl or butynyl), C₂₋₆ alkenyl (e.g., C₂₋₄ alkenyl, such as vinyl, propenyl, or butenyl), optionally deuterated C₃₋₆ cycloalkyl (e.g., optionally perdeuterated cyclopropyl, optionally perdeuterated cyclobutyl, optionally perdeuterated cyclopentyl, or optionally perdeuterated cyclohexyl), R^{c12}—R^{c11}, and any combination thereof, wherein R^{c11} represents optionally substituted C₁₋₆ alkylene (e.g., optionally substituted C₁₋₄ alkylene, such as optionally substituted methylene, ethylene, or propylene), or optionally substituted C₂₋₆ alkenylene (e.g., optionally substituted C₂₋₄ alkenylene, such as optionally substituted vinylene, propenylene, butenylene), and R^{c12} represents halogen (e.g., fluorine, chlorine, bromine, or iodine), R^{c13}R^{c14}N—, hydroxy, carboxyl, ethynyl, or vinyl, wherein R^{c13} and R^{c14} are the same or different and independently represent hydrogen or C₁₋₃ alkyl (e.g., methyl, ethyl, or propyl); and the C₁₋₆ alkylene and C₂₋₆ alkenylene are each independently optionally substituted with one or more substituents selected from the group consisting of deuterium, halogen, hydroxy, mercapto, cyano, amino, nitro, C₁₋₆ alkyl, halogenated C₁₋₆ alkyl, deuterated C₁₋₆ alkyl, C₁₋₆ alkoxy, and halogenated C₁₋₆ alkoxy.

In some embodiments, the R_{c} of the compound of Formula (I) represents the following groups: a bond, —O—, —NHC(O)—*, —C(O)NH—*, —C(O)O—*, —OC(O)—*, —CH₂C(O)O—*, —CH₂OC(O)—*, —NH—, —N(CH₃)—, —N(CH₃)—(CH₂)₂—N(CH₃)—*, —N(CH₃)—(CH₂)₃—N(CH₃)—*, —NH—(CH₂)₂—N(CH₃)—*, —N(CH₃)—(CH₂)₃—NH—*, —CH₂—NHC(O)—*, —CH₂—C(O)NH—*, —CH₂—NH—*, —O—(CH₂)₂—N(CH₃)—*, —O—(CH₂)₂—NH—*, or —CH₂—N(CH₃)—*, or

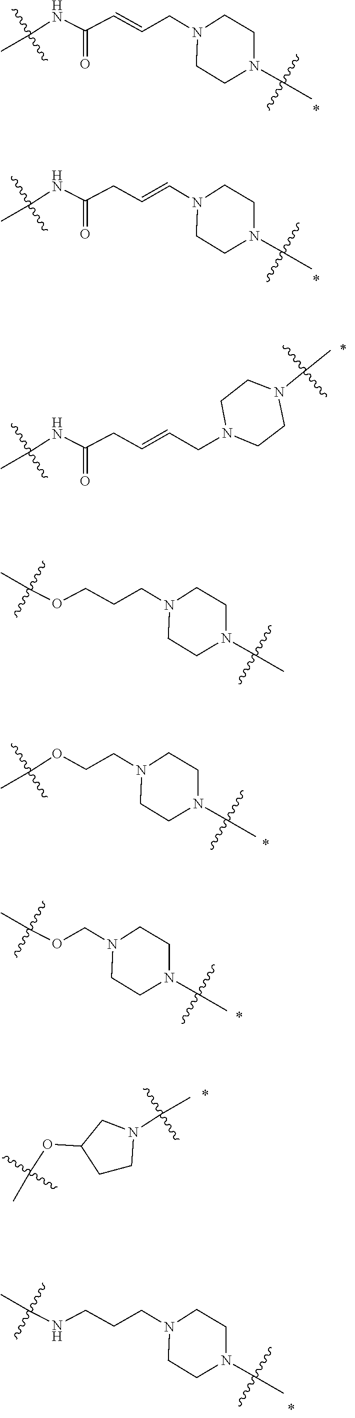

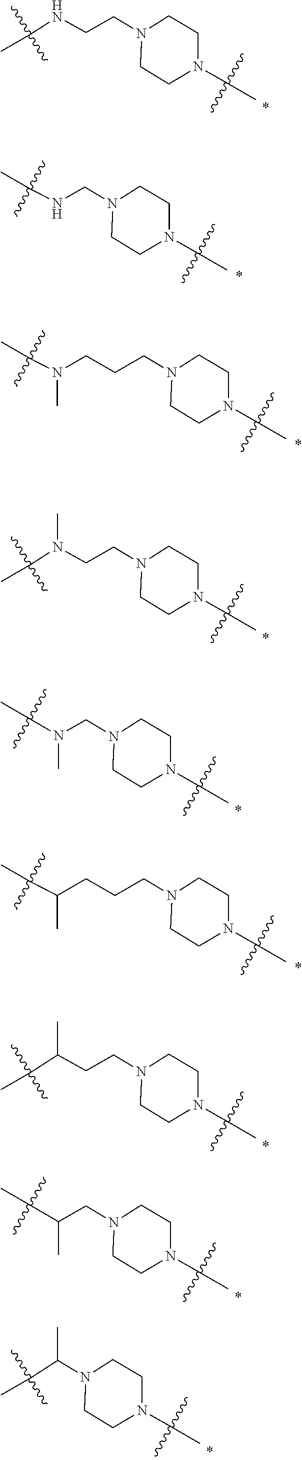

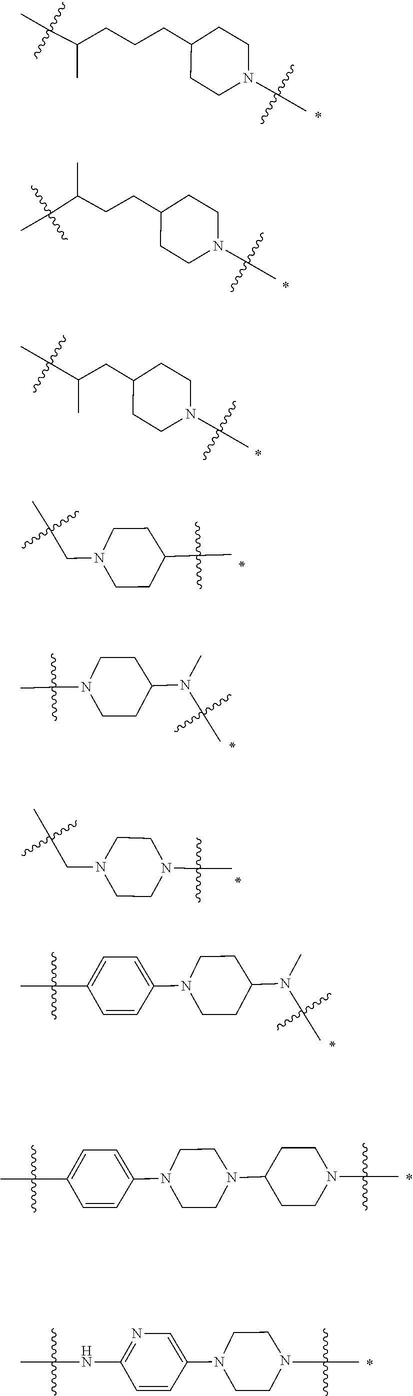

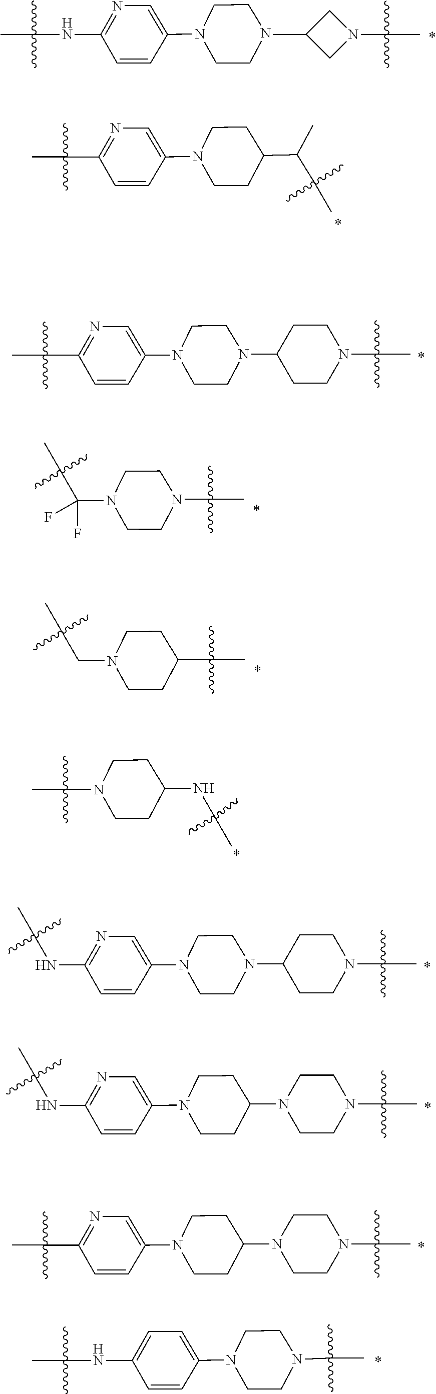

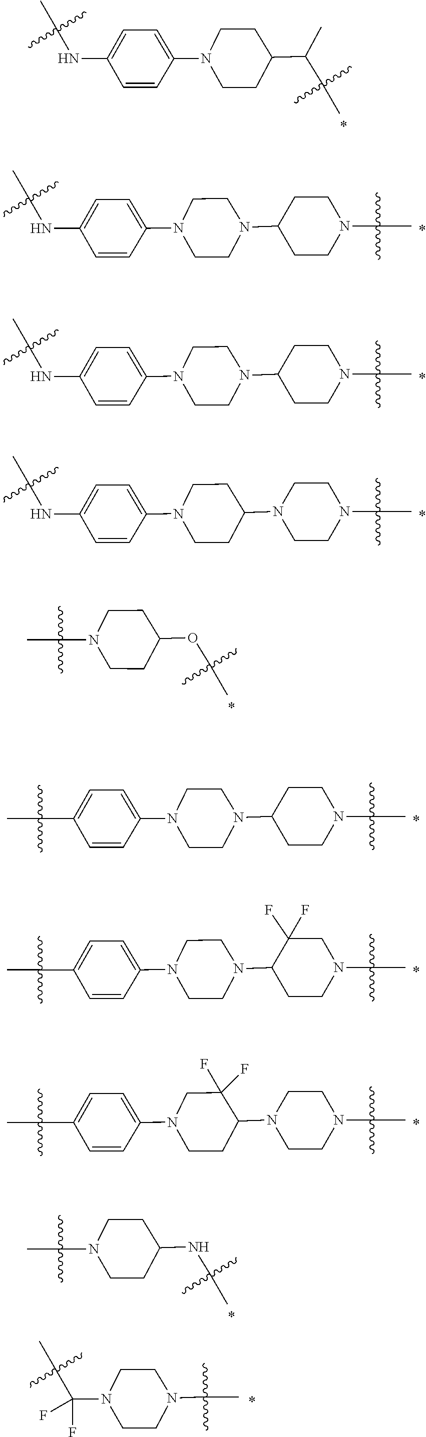

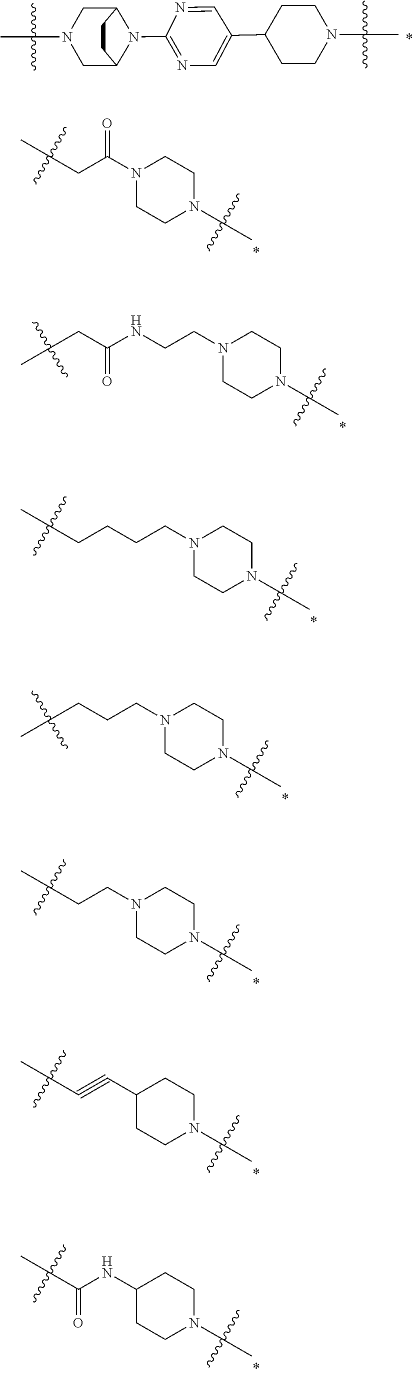

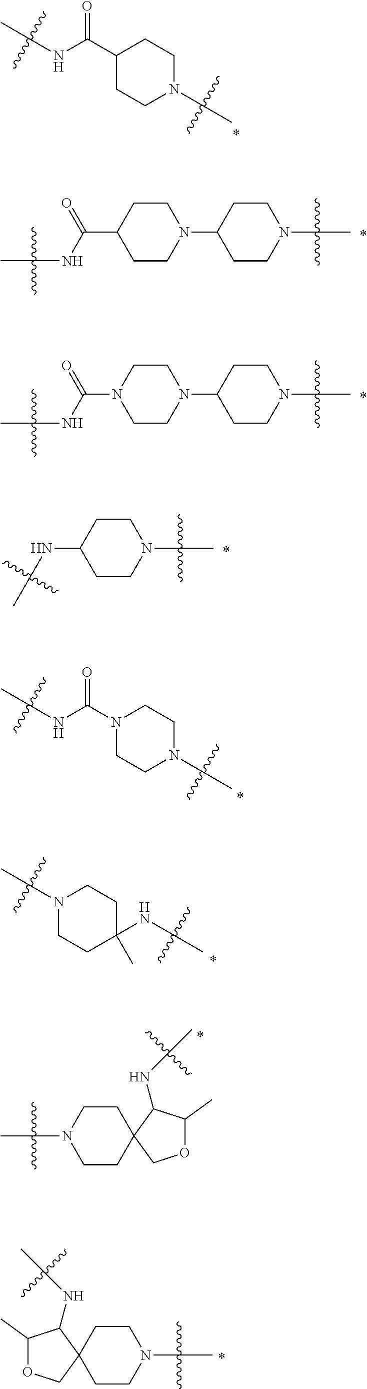

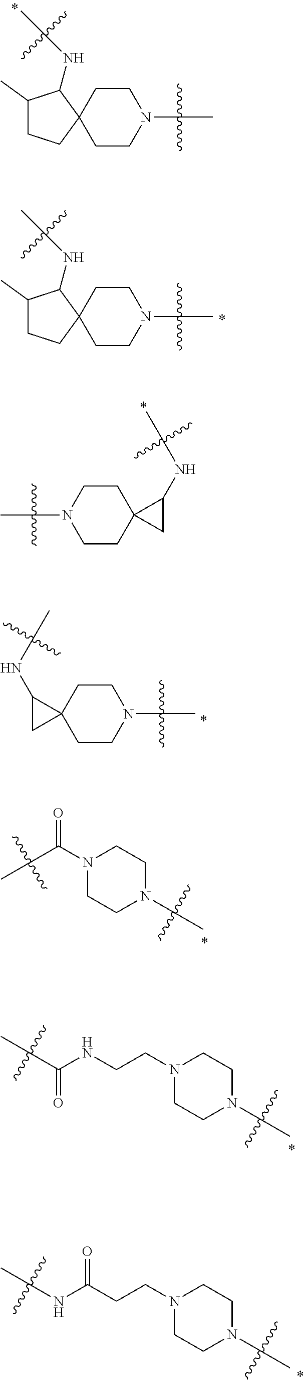

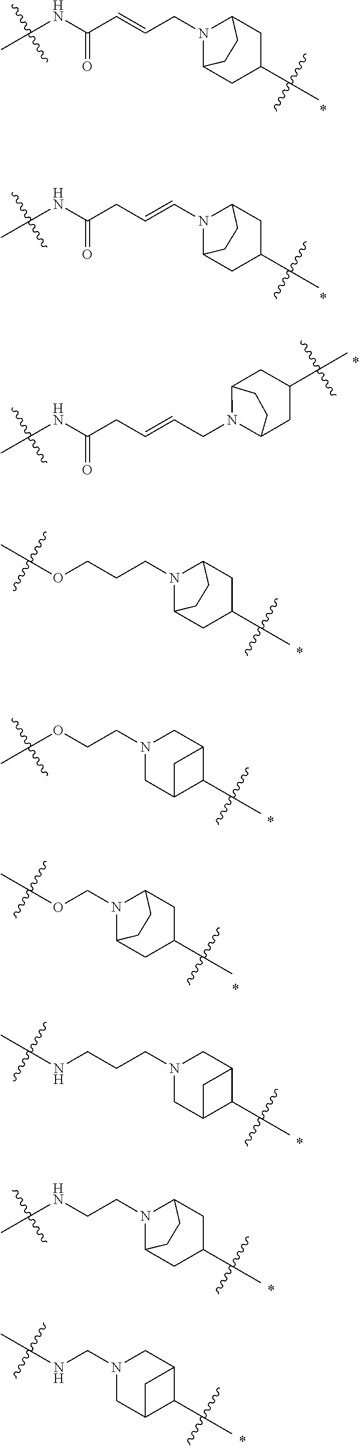

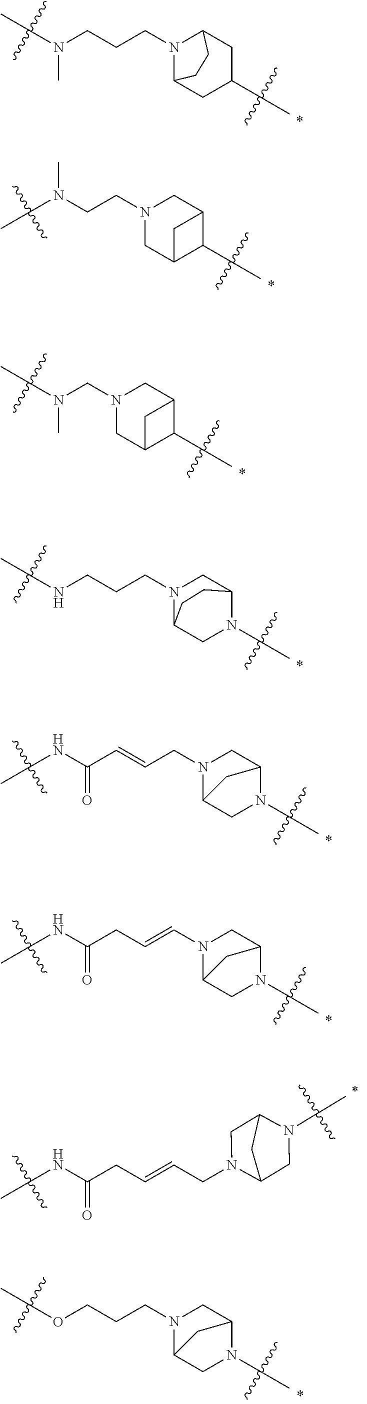

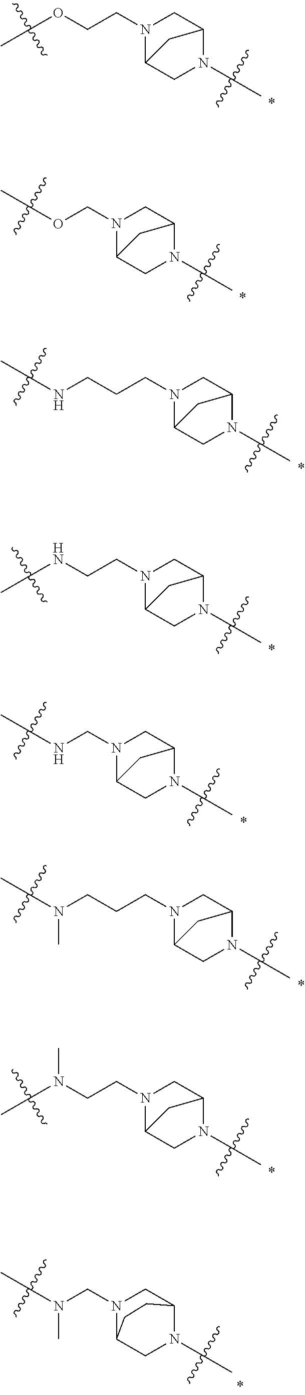

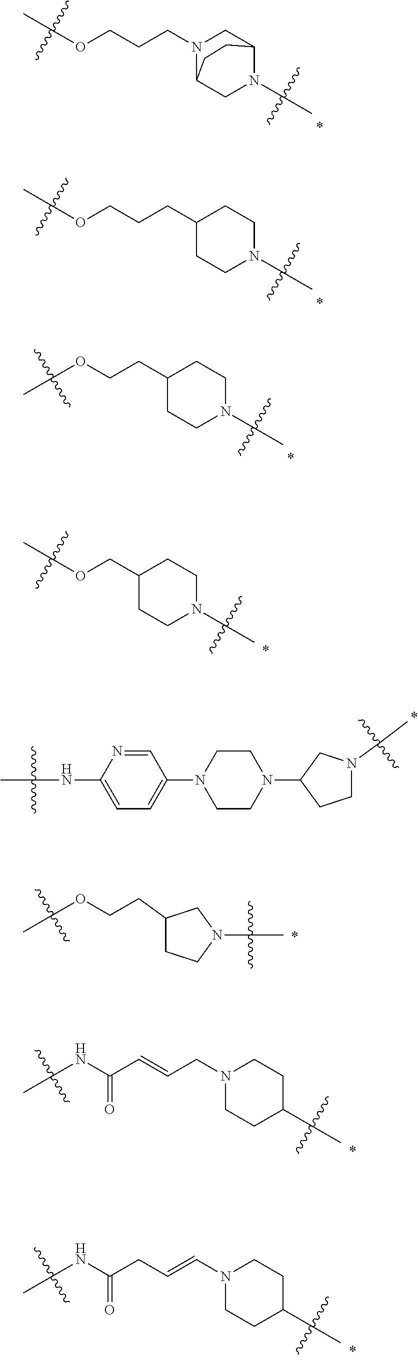

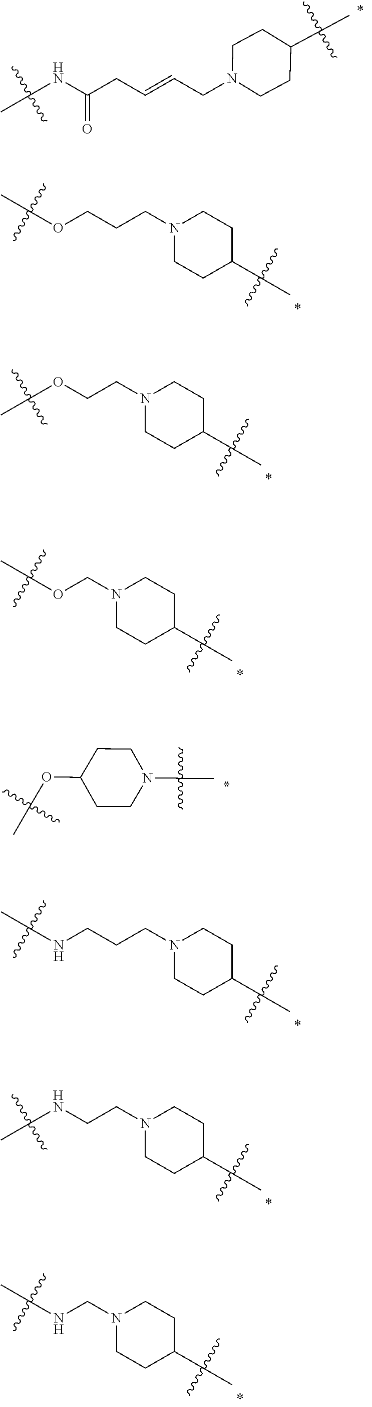

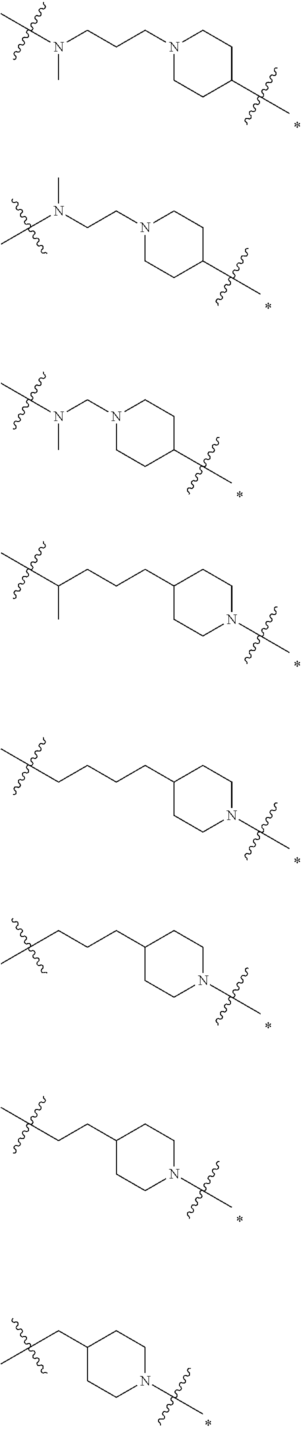

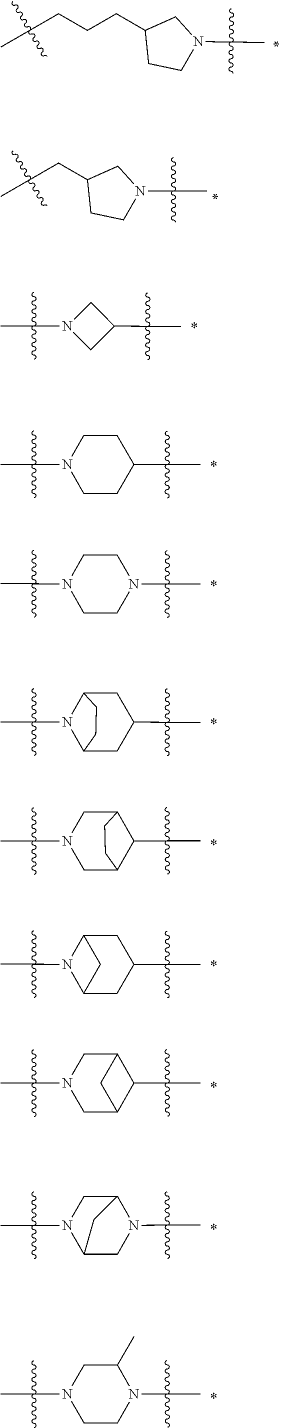

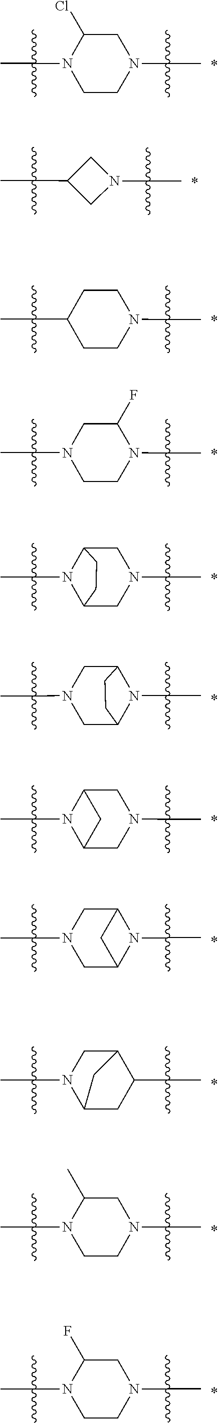

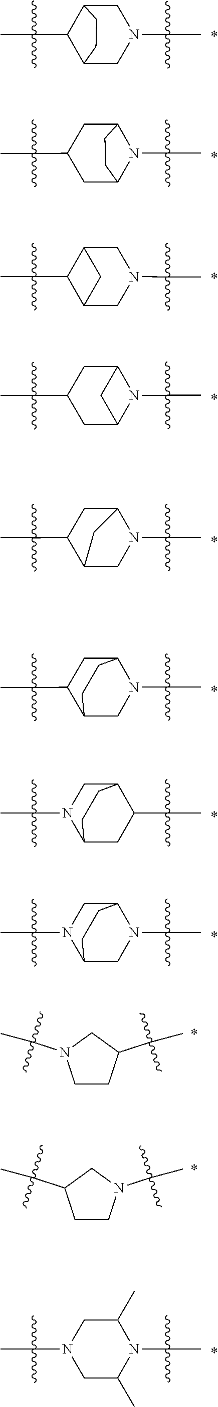

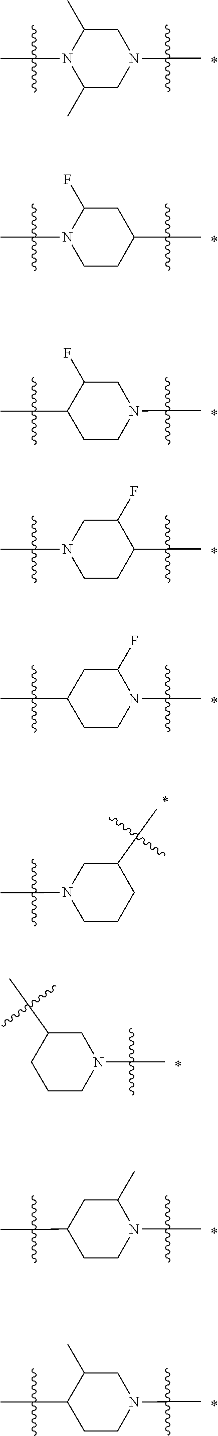

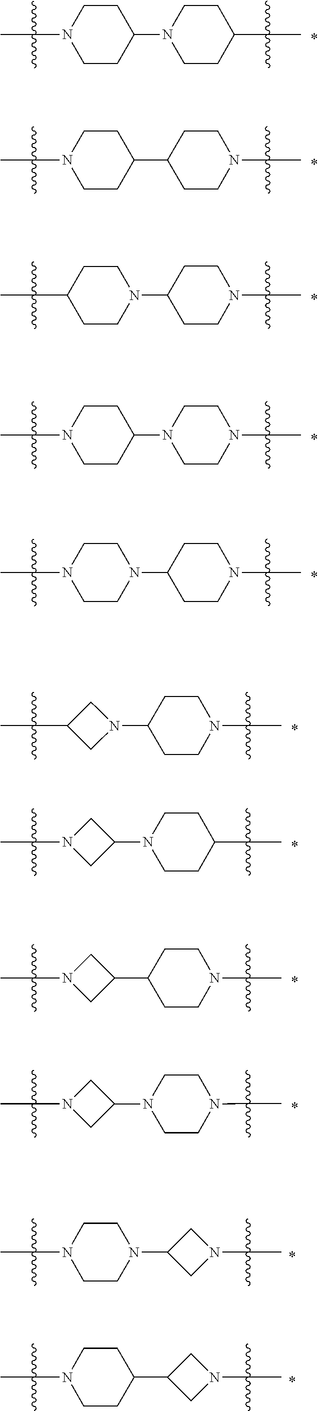

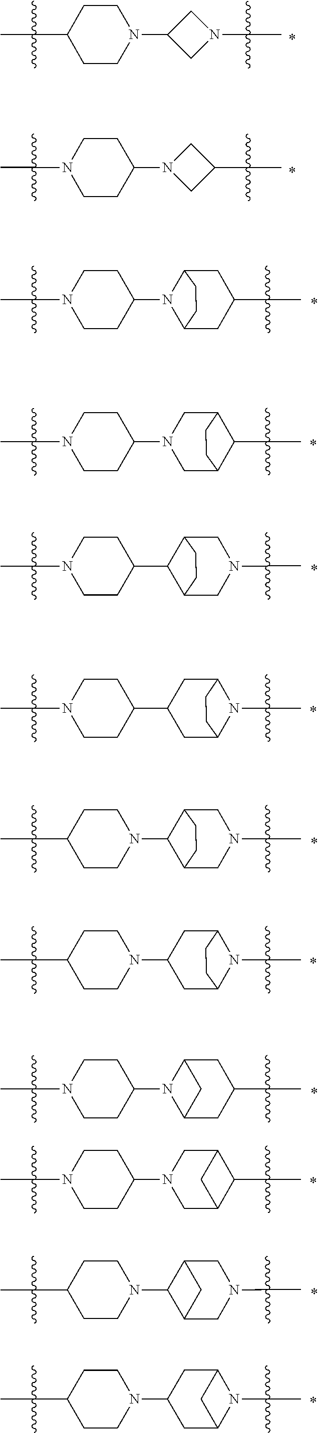

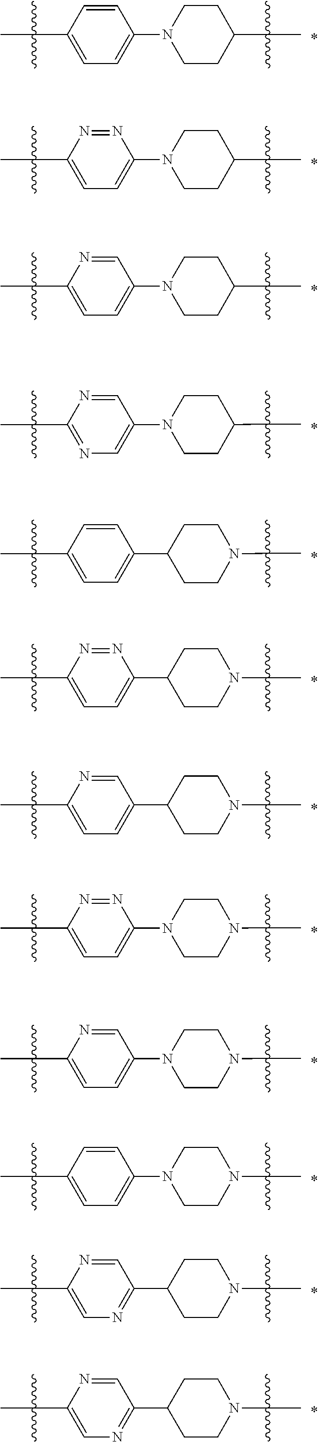

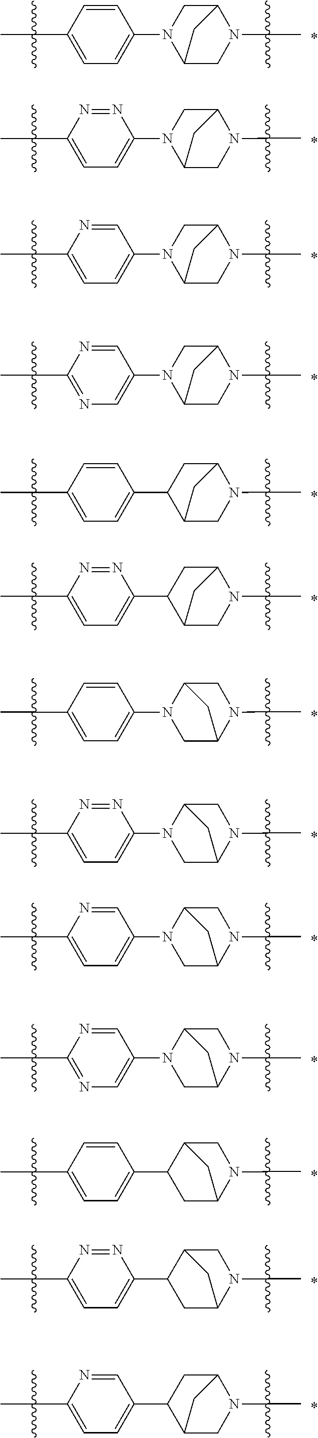

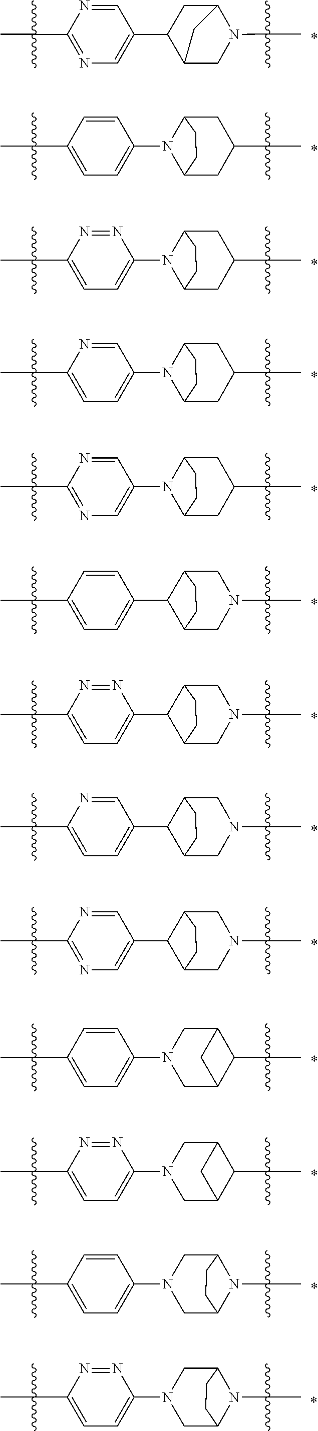

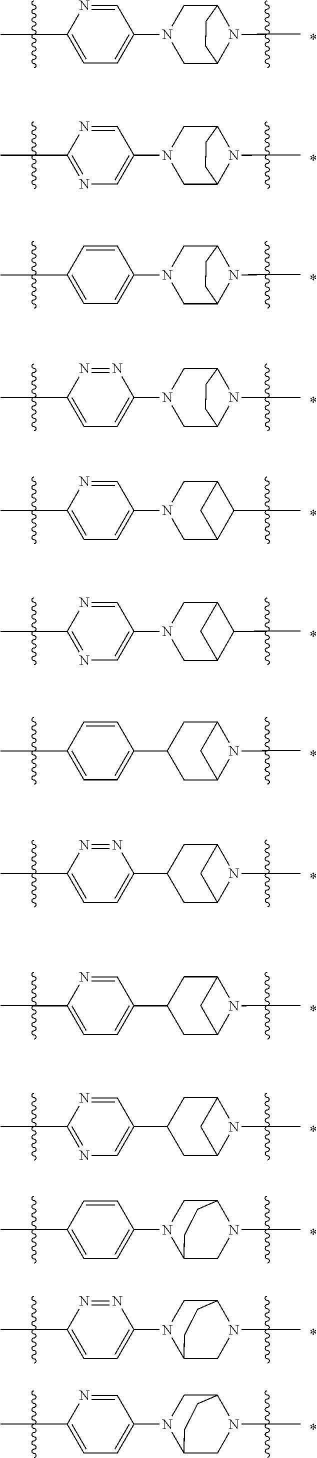

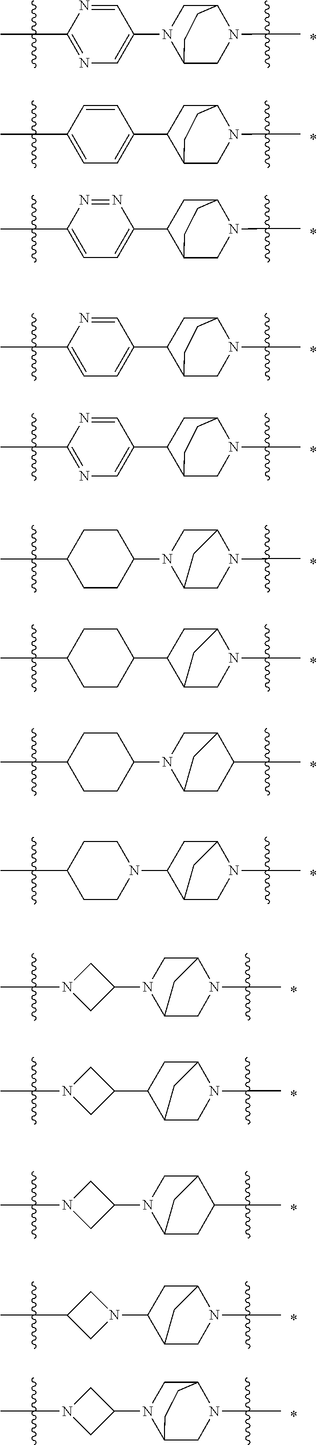

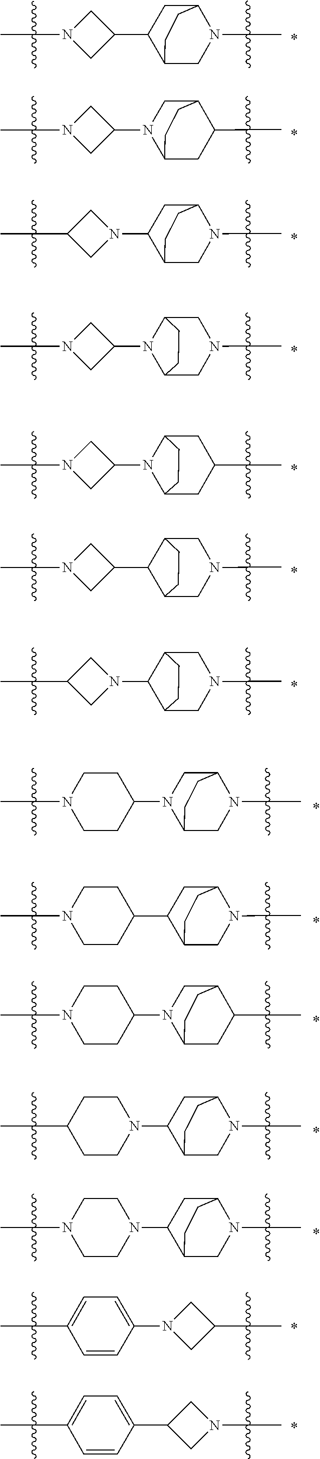

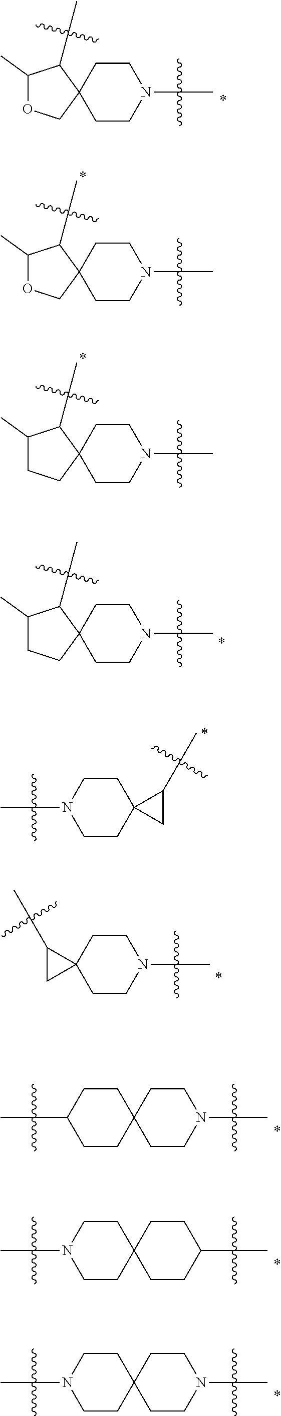

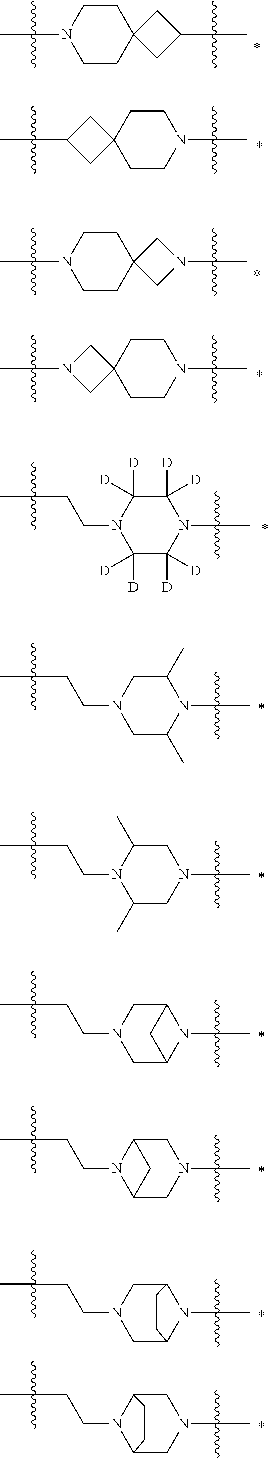

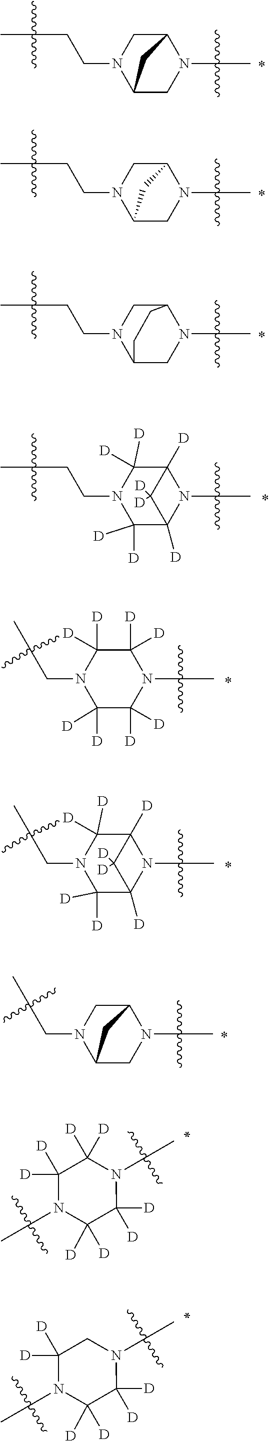

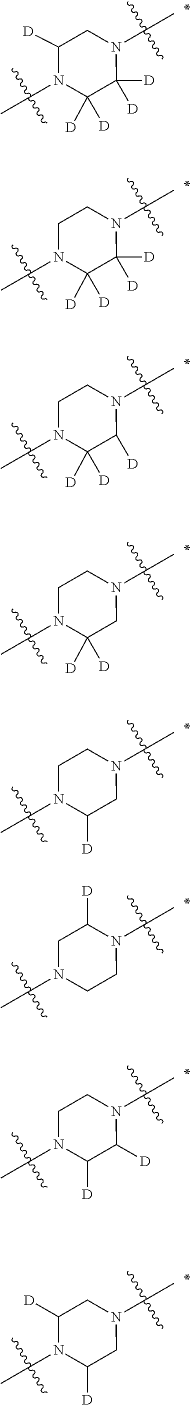

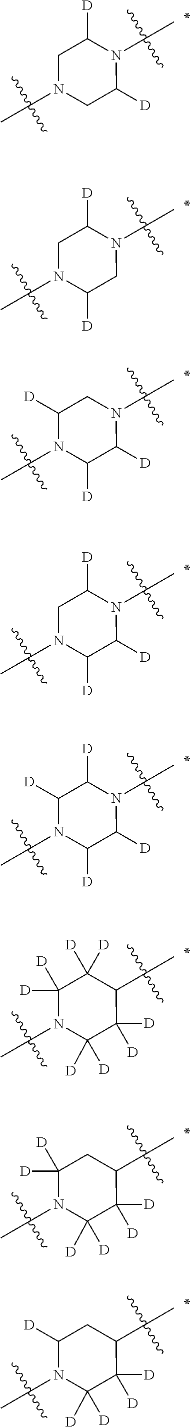

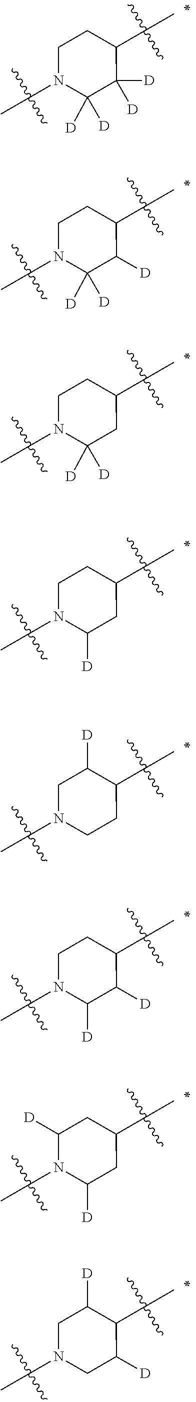

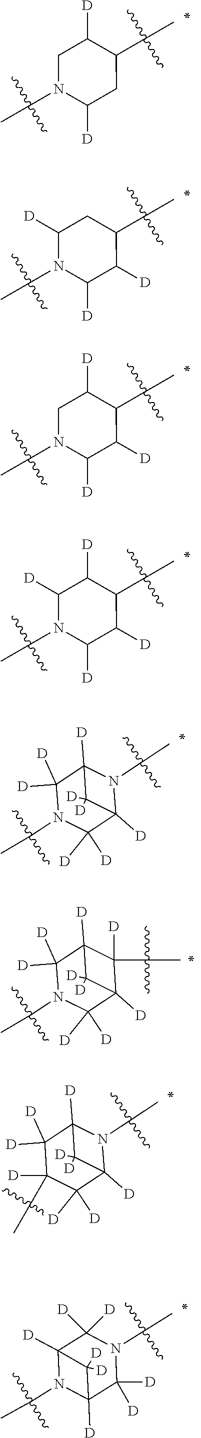

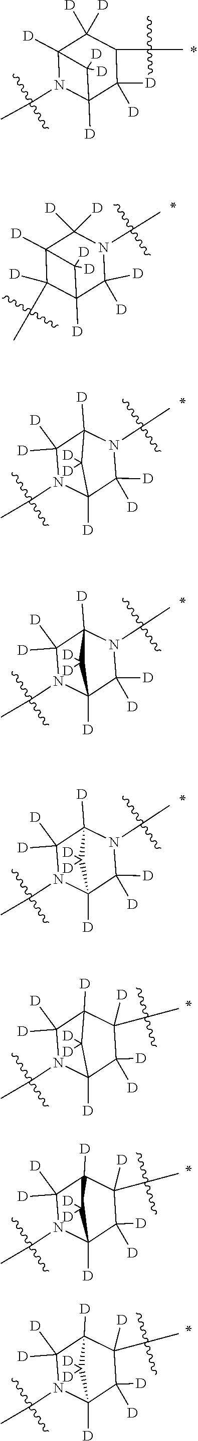

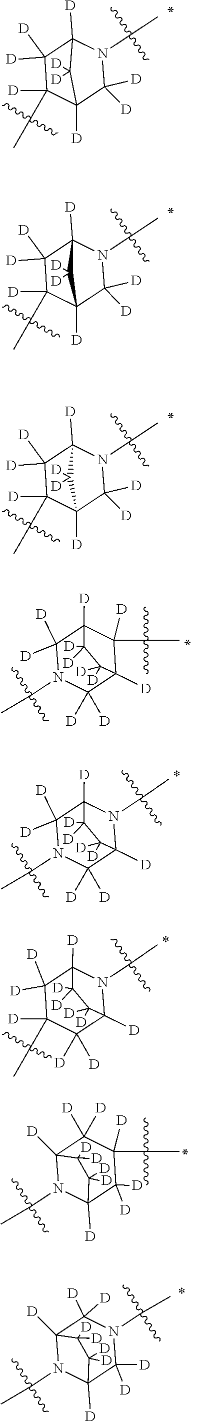

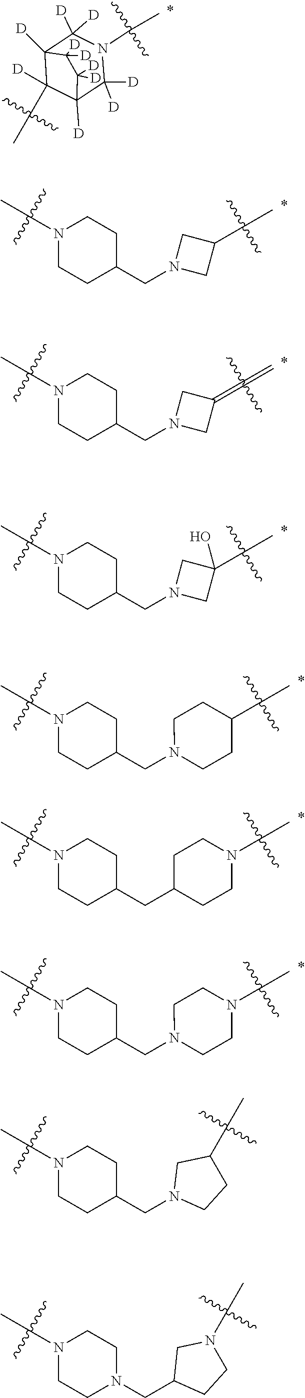

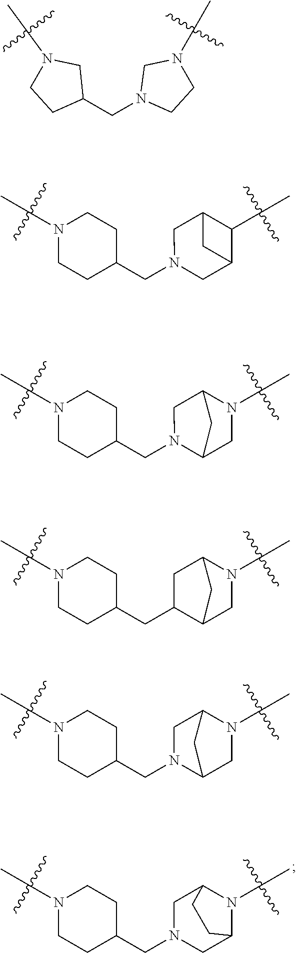 [SMILES: *N1CCC(CN2CCN(*)C2)C1 ; *C1C2CC1CN(CC1CCN(*)CC1)C2 ; *N1CCC(CN2CC3CC2CN3*)CC1 ; *N1CCC(CC2CC3CC2CN3*)CC1 ; *N1CCC(CN2CC3CC2CN3*)CC1 ; *N1CCC(CN2CC3CCC(C2)N3*)CC1]

wherein the groups are each independently optionally substituted with one or more (e.g., 1-5, such as 1, 2, 3, 4, or 5) substituents selected from the group consisting of deuterium, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), C₁₋₆ alkoxy (e.g., C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), halogen (e.g., fluorine, chlorine, bromine, or iodine), amino, hydroxy, mercapto, cyano, C₂₋₆ alkynyl (e.g., C₂₋₄ alkynyl, such as ethynyl, propynyl or butynyl), C₂₋₆ alkenyl (e.g., C₂₋₄ alkenyl, such as vinyl, propenyl, or butenyl), optionally deuterated C₃₋₆ cycloalkyl (e.g., optionally perdeuterated cyclopropyl, optionally perdeuterated cyclobutyl, optionally perdeuterated cyclopentyl, or optionally perdeuterated cyclohexyl), R^{c12}—R^{c11}—, and any combination thereof; wherein R^{c11} represents optionally substituted C₁₋₆ alkylene (e.g., optionally substituted C₁₋₄ alkylene, such as optionally substituted methylene, ethylene, or propylene) or optionally substituted C₂₋₆ alkenylene (e.g., optionally substituted C₂₋₄ alkenylene, such as optionally substituted vinylene, propenylene, butenylene), and R^{c12} represents halogen (e.g., fluorine, chlorine, bromine, or iodine), R^{c13}R^{c14}N—, hydroxy, carboxyl, ethynyl, or vinyl, wherein R^{c13} and R^{c14} are the same or different and independently represent hydrogen or C₁₋₃ alkyl (e.g., methyl, ethyl, or propyl); and the C₁₋₆ alkylene and C₂₋₆ alkenylene are each independently optionally substituted with one or more deuterium, halogen, hydroxy, mercapto, cyano, amino, nitro, C₁₋₆ alkyl, halogenated C₁₋₆ alkyl, deuterated C₁₋₆ alkyl, C₁₋₆ alkoxy, or halogenated C₁₋₆ alkoxy; and
symbol * indicates the point of attachment to LIN.

In some embodiments, the ULM represents a ligand moiety capable of binding to an E3 ubiquitin ligase of the compound of Formula (I), and represents the structure of the following Formula (ULM):

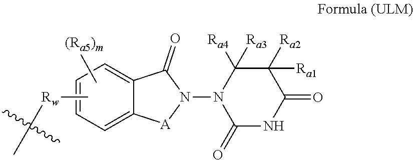

wherein A represents CO, CH₂, or CD₂;
Rₐ₁, Rₐ₂, Rₐ₃, and Rₐ₄ are the same or different and independently represent hydrogen, deuterium, halogen (e.g., fluorine, chlorine, bromine, or iodine), C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), C₁₋₆ alkoxy (e.g., C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), or deuterated C₁₋₆ alkoxy;
(Rₐ₅)ₘ indicates that the isoindoline ring to which it is attached is optionally substituted with m Ras, with each Ras being the same or different and each independently representing halogen (e.g., fluorine, chlorine, bromine, or iodine), hydroxy, mercapto, nitro, amino, cyano, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), C₁₋₆ alkoxy (e.g., C₁₋₄ alkoxy, such as methoxy, ethoxy, propoxy, isopropoxy, n-butoxy, sec-butoxy, or tert-butoxy), deuterated C₁₋₆ alkoxy, halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), C₂₋₆ alkenyl, or C₂₋₆ alkynyl;
m represents an integer of 0, 1, 2, 3, or 4;
R_{w} represents O, S, S(O), S(O)₂, alkenylene (e.g., C₂₋₆ alkenylene), alkynylene (e.g., C₂₋₆ alkynylene), or N(Rₐ₆), where Rₐ₆ represents H or C₁₋₃ alkyl; or
R_{w} represents a bond; or
R_{w} represents:

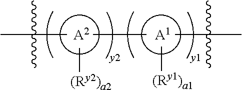

wherein each ring A¹ is the same or different and independently represents nitrogen-containing heterocyclylene (e.g., 4-to 20-membered nitrogen-containing heterocyclylene), arylene (e.g., C₅₋₂₀ arylene), or heteroarylene (e.g., 5-to 20-membered heteroarylene), y1 represents an integer of 1 or 2, and (R^{y1})ₐ₁ indicates that each ring A¹ is independently optionally substituted with a1 R^{y1} substituents, with each R^{y1} independently representing deuterium, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), C₁₋₆ alkoxy (e.g., C₁₋₄ alkoxy, such as methoxy, ethoxy, propoxy, isopropoxy, n-butoxy, sec-butoxy, or tert-butoxy), halogen (e.g., fluorine, chlorine, bromine, or iodine), amino, hydroxy, mercapto, cyano, carboxyl, C₂₋₆ alkynyl, or C₂₋₆ alkenyl, and a1 represents an integer of 0-20, and
each ring A² is the same or different and independently represents heterocyclylene (e.g., 4-to 20-membered heterocyclylene), cycloalkylene (e.g., C₃₋₂₀cycloalkylene), arylene (e.g., C₅₋₂₀ arylene), or heteroarylene (e.g., 5-to 20-membered heteroarylene), y2 represents an integer of 0 or 1, and (R^{y2})ₐ₂ indicates that each ring A² is independently optionally substituted with a2 R^{y2} substituents, with each R^{y2} independently representing deuterium, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), C₁₋₆ alkoxy (e.g., C₁₋₄ alkoxy, such as methoxy, ethoxy, propoxy, isopropoxy, n-butoxy, sec-butoxy, or tert-butoxy), halogen (e.g., fluorine, chlorine, bromine, or iodine), amino, hydroxy, mercapto, cyano, carboxyl, C₂₋₆ alkynyl, or C₂₋₆ alkenyl, and a2 represents an integer of 0-20.

In some embodiments, A of the compound of Formula (I) represents CO. In some embodiments, A of the compound of Formula (I) represents CH₂. In some embodiments, A of the compound of Formula (I) represents CD₂.

In some embodiments, Rₐ₁, Rₐ₂, Rₐ₃, and Rₐ₄ of the compound of Formula (I) represent hydrogen.

In some embodiments, R_{w} of the compound of Formula (I) represents a bond.

In some embodiments, R_{w} of the compound of Formula (I) represents O, S, S(O), S(O)₂, alkenylene (e.g., C₂₋₆alkenylene), alkynylene (e.g., C₂₋₆alkynylene), or N(Rₐ₆), wherein Rₐ₆ represents H or C₁₋₃ alkyl.

In some embodiments, R_{w} of the compound of Formula (I) represents:

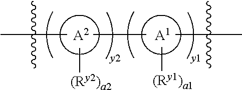

wherein each ring A¹ is the same or different and independently represents 4-to 20-membered (e.g., 4-to 18-membered, 4-to 15-membered, 4-to 12-membered, 4-to 11-membered, 4-to 10-membered, 4-to 9-membered, 4-to 8-membered, 4-to 7-membered, 4-to 6-membered, 5-to 15-membered, or 5-to 9-membered) nitrogen-containing heterocyclylene, C₅₋₂₀ arylene (e.g., C₆₋₂₀ arylene, C₅₋₁₅ arylene, or C₆₋₁₅ arylene), or 5-to 20-membered heteroarylene (e.g., 5-to 15-membered, 5-to 10-membered, 5-to 9-membered, 5-to 8-membered, 5-to 7-membered, or 5-to 6-membered heteroarylene). In some embodiments, 4-to 20-membered nitrogen-containing heterocyclylene is a 4-to 20-membered (e.g., 4-to 15-membered, 4-to 12-membered, 4-to 11-membered, 4-to 10-membered, 4-to 9-membered, 4-to 8-membered, 4-to 7-membered, 4-to 6-membered, 5-to 15-membered, or 5-to 9-membered) heterocyclylene containing one nitrogen atom and optionally containing a heteroatom(s) selected from nitrogen, oxygen, and sulfur. Examples of the 4-to 20-membered nitrogen-containing heterocyclylene include, but are not limited to, e.g., piperidinylene, piperazinylene, morpholinylene, azetidinylene, pyrrolidinylene, imidazolidylene, pyrazolidylene, oxazolidinylene, thiazolidinylene, thiomorpholinylene, azepanylene, diazacycloheptanylene, azacyclooctylene, diazacyclooctylene, azabicyclo[3.1.1]heptanylene, azabicyclo[2.2.1]heptanylene, azabicyclo[3.2.1]octanylene, azabicyclo[2.2.2]octanylene, diazabicyclo[3.1.1]heptanylene, diazabicyclo[2.2.1]heptanylene, diazabicyclo[3.2.1]octanylene, diazabicyclo[2.2.2]octanylene, 3-azaspiro[5.5]undecanylene, 8-azaspiro[4.5]decanylene, 2-oxa-8-azaspiro[4.5]decanylene, and 3-methyl-2-oxa-8-azaspiro[4.5]decanylene. In some embodiments, examples of the 5-to 20-membered arylene include, but are not limited to, e.g., phenylene or naphthylene. In some embodiments, the 5-to 20-membered heteroarylene is a 5-to 20-membered (e.g., 5-to 15-membered, 5-to 12-membered, 5-to 9-membered, 5-to 8-membered, 5-to 7-membered, or 5-to 6-membered) heteroarylene containing one nitrogen atom and optionally containing a heteroatom(s) selected from nitrogen, oxygen, and sulfur. In some embodiments, examples of the 5-to 20-membered heteroarylene include, but are not limited to, e.g., oxazolylene, isoxazolylene, oxadiazolylene, thienylene, thiazolylene, isothiazolylene, thiadiazolylene, pyrrolylene, imidazolylene, pyrazolylene, triazolylene, pyridylene, pyrimidinylene, pyridazinylene, pyrazinylene, indolylene, isoindolylene, benzofuranylene, isobenzofuranylene, benzothienylene, indazolylene, benzimidazolylene, benzoxazolylene, benzisoxazolylene, benzothiazolylene, benzisothiazolylene, benzotriazolylene, benzo[2,1,3]oxadiazolylene, benzo[2,1,3]thiadiazolylene, benzo[1,2,3]thiadiazolylene, quinolinylene, isoquinolinylene, naphthyridinylene, cinnolinylene, quinazolinylene, quinoxalinylene, phthalazinylene, pyrazolo[1,5-a]pyridylene, pyrazolo[1,5-a]pyrimidinylene, imidazo[1,2-a]pyridylene, 1H-pyrrolo[3,2-b]pyridylene, 1H-pyrrolo[2,3-b]pyridylene, pyrrolo[2,1-b]thiazolylene, and imidazo[2,1-b]thiazolylene. Each ring A¹ is independently optionally substituted with a1 R^{y1} substituents, and each R^{y1} independently represents deuterium, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), C₁₋₆ alkoxy (e.g., C₁₋₄ alkoxy, such as methoxy, ethoxy, propoxy, isopropoxy, n-butoxy, sec-butoxy, or tert-butoxy), halogen (e.g., fluorine, chlorine, bromine, or iodine), amino, hydroxy, mercapto, cyano, carboxyl, C₂₋₆ alkynyl, or C₂₋₆ alkenyl. a1 represents an integer of 0-20, e.g., an integer of 0, 1, 2, 3, 4, 5, 6, 7, 8, 9, 10, 11, 12, 13, 14, 15, or 20. The number of substituents is not theoretically limited in any way, or is automatically limited by the size of the building units.

In some embodiments, y1 of the compound of Formula (I) represents an integer of 1.

In some embodiments, y1 of the compound of Formula (I) represents an integer of 2.

In some embodiments, each ring A¹ of the compound of Formula (I) is the same or different and independently represents the following optionally substituted groups:

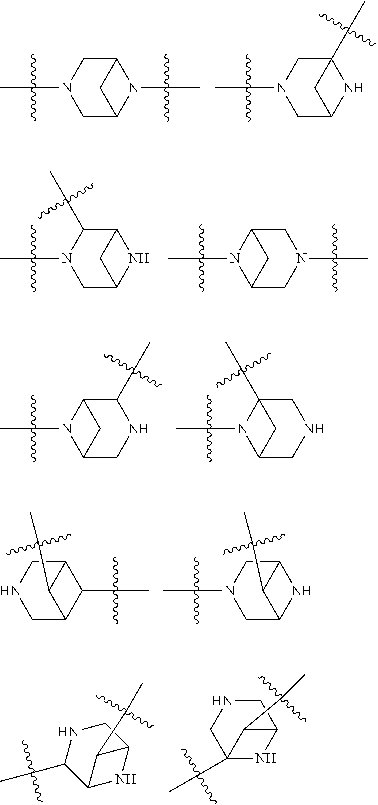

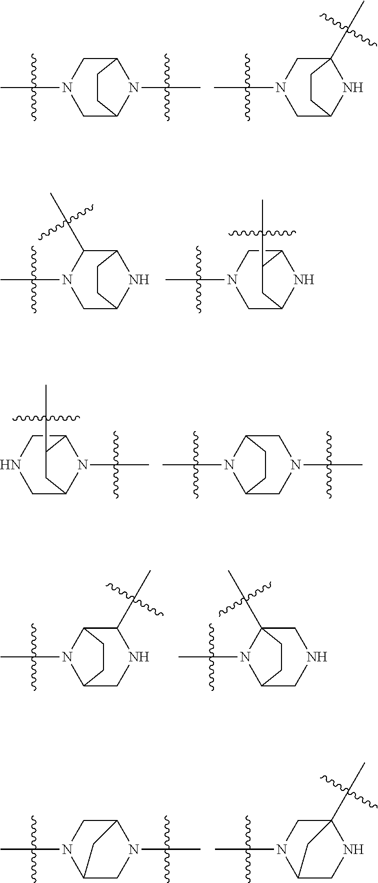

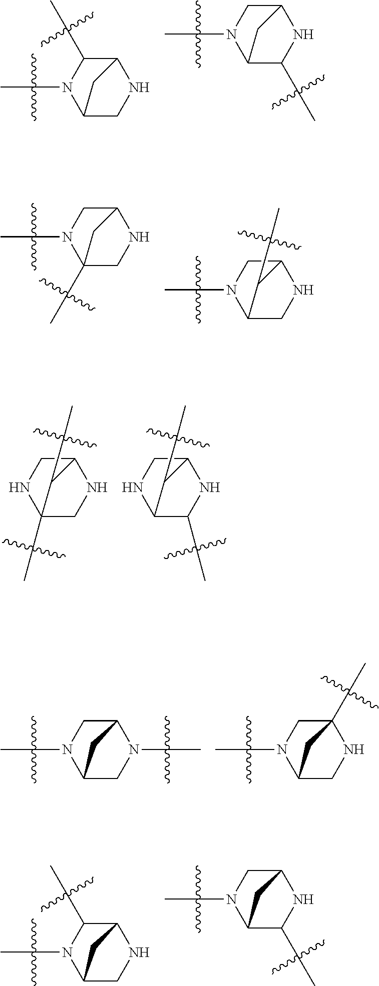

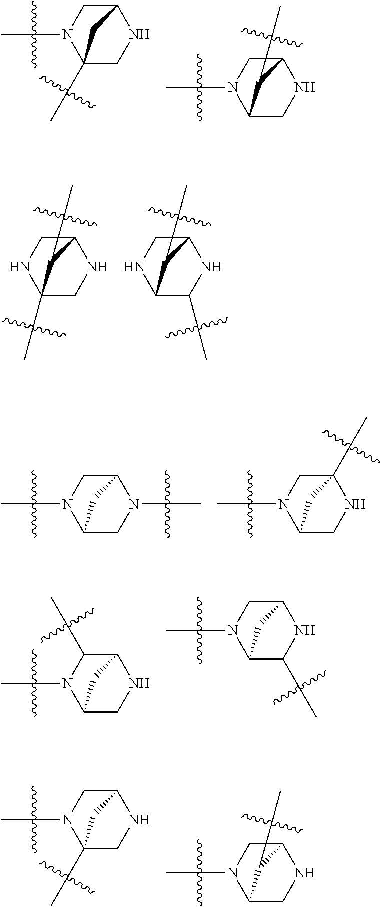

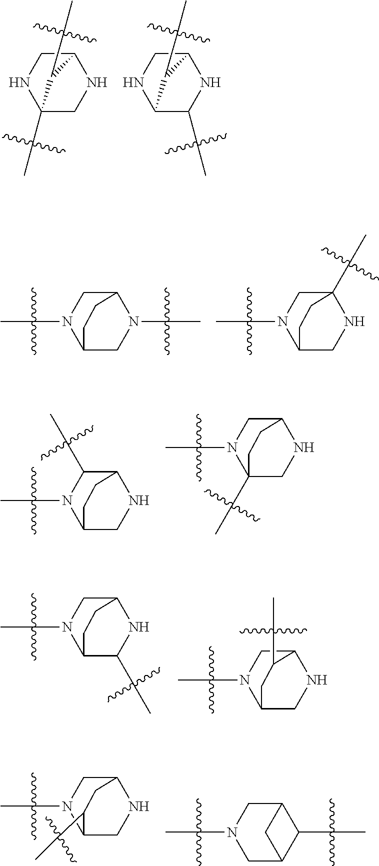

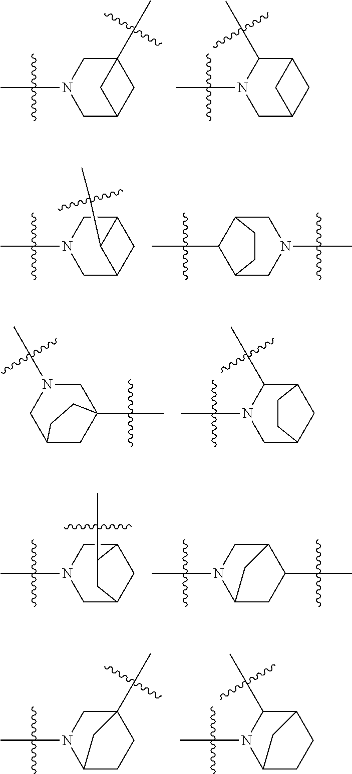

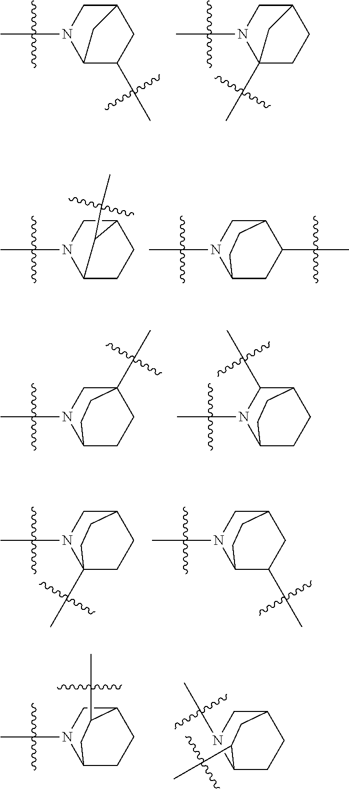

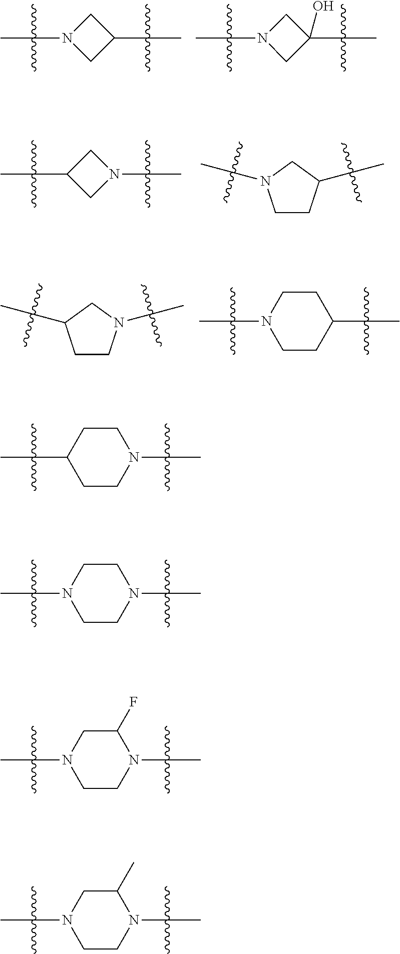

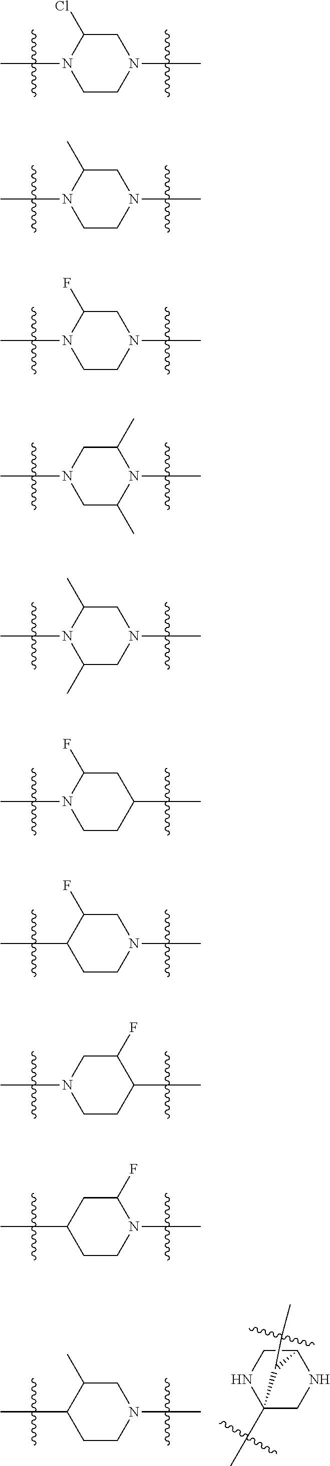

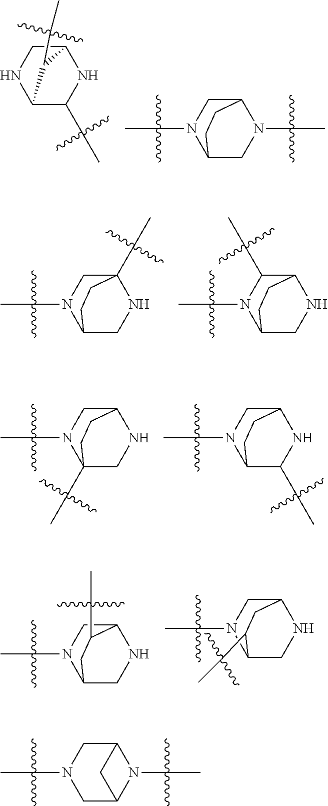

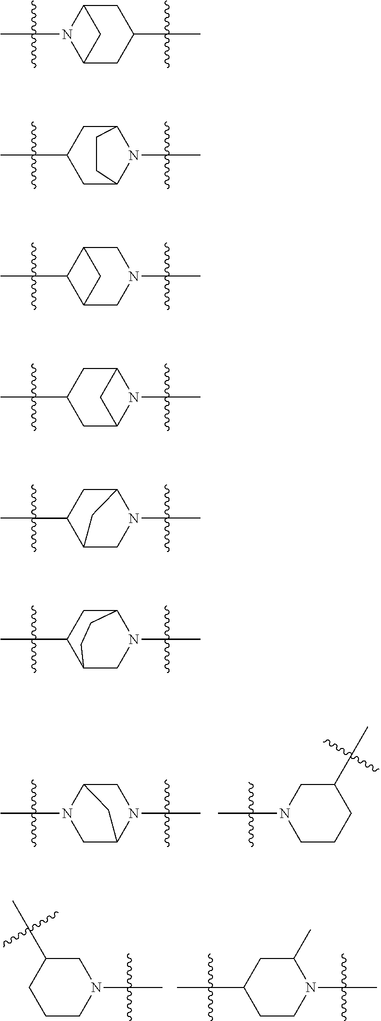

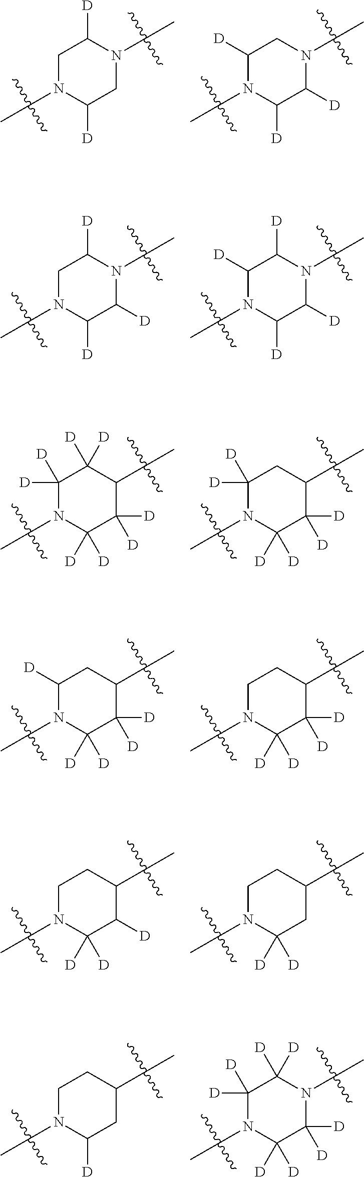

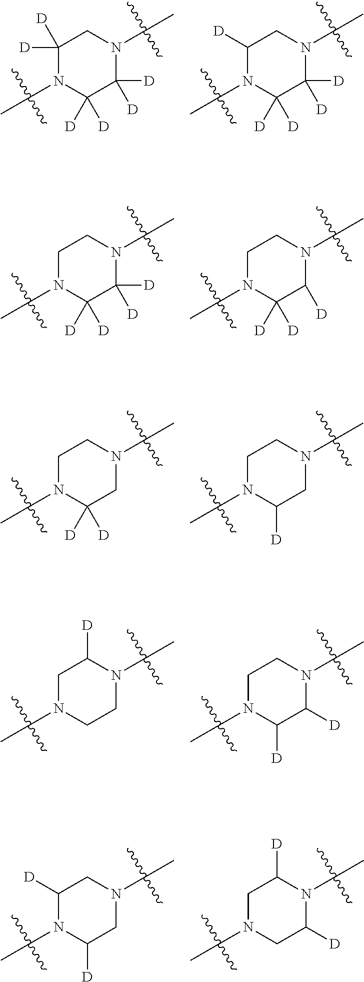

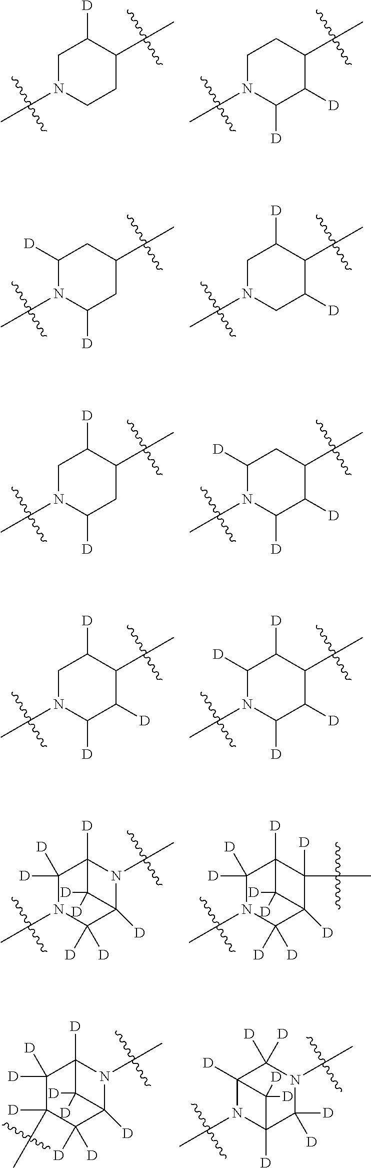

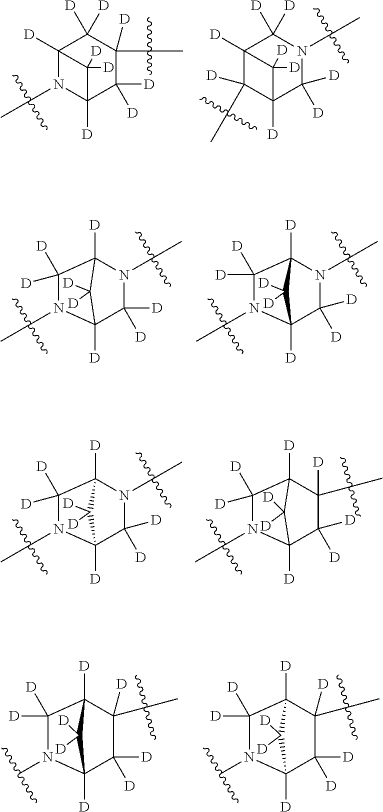

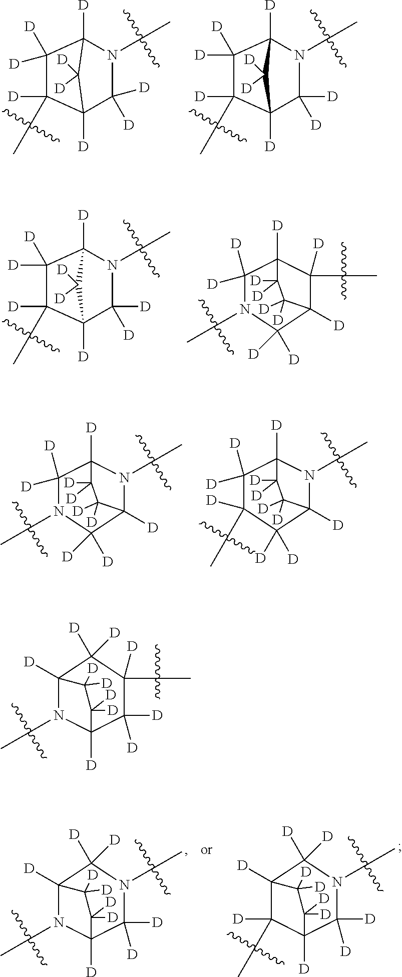

each ring A¹ is independently optionally substituted with a1 R^{y1} substituents, and each R^{y1} independently represents deuterium, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), C₁₋₆ alkoxy (e.g., C₁₋₄ alkoxy, such as methoxy, ethoxy, propoxy, isopropoxy, n-butoxy, sec-butoxy, or tert-butoxy), halogen (e.g., fluorine, chlorine, bromine, or iodine), amino, hydroxy, mercapto, cyano, carboxyl, C₂₋₆ alkynyl (e.g., C₂₋₄ alkynyl, such as ethynyl, propynyl or butynyl), or C₂₋₆ alkenyl (e.g., C₂₋₄ alkenyl, such as vinyl, propenyl, or butenyl). a1 represents an integer of 0-20, e.g., an integer of 0, 1, 2, 3, 4, 5, 6, 7, 8, 9, 10, 11, 12, 13, 14, 15, or 20. The number of substituents is not theoretically limited in any way, or is automatically limited by the size of the building units.

In some embodiments, each ring A² of the compound of Formula (I) is the same or different and independently represents 4-to 20-membered (e.g., 4-to 18-membered, 4-to 15-membered, 4-to 12-membered, 4-to 11-membered, 4-to 10-membered, 4-to 9-membered, 4-to 8-membered, 4-to 7-membered, 4-to 6-membered, 5-to 15-membered, or 5-to 9-membered) heterocyclylene, C₃₋₂₀cycloalkylene (e.g., C₃-15cycloalkylene, or C₃₋₁₁cycloalkylene), C₅₋₂₀ arylene (e.g., C₆₋₂₀ arylene, C₅₋₁₅ arylene, or C₆₋₁₅ arylene), or 5-to 20-membered heteroarylene (e.g., 5-to 15-membered, 5-to 10-membered, 5-to 9-membered, 5-to 8-membered, 5-to 7-membered, or 5-to 6-membered heteroarylene). In some embodiments, the 4-to 20-membered heterocyclylene is a 4-to 20-membered (e.g., 4-to 15-membered, 4-to 12-membered, 4-to 11-membered, 4-to 10-membered, 4-to 9-membered, 4-to 8-membered, 4-to 7-membered, 4-to 6-membered, 5-to 15-membered, or 5-to 9-membered) saturated or partially unsaturated (i.e., containing one or more double bonds, but not having a fully conjugated x-electron system) divalent monocyclic, bicyclic, tricyclic or polycyclic cyclic hydrocarbon group containing one or more (e.g., from 1 to 5, from 1 to 4, or from 1 to 3, or from 1 to 2, or 1) heteroatoms selected from the group consisting of oxygen, nitrogen, and sulfur. Examples of the 4-to 20-membered heterocyclylene include, but are not limited to, monocyclic heterocyclylene (e.g., 4-to 20-membered monocyclic heterocyclylene), bridged heterocyclylene (e.g., 5-to 20-membered bridged heterocyclylene, or 7-to 15-membered bridged heterocyclylene), fused heterocyclylene, and spiro-heterocyclylene (e.g., 5-to 20-membered spiro-heterocyclylene), e.g,, azetidinylene, oxetanylene, pyrrolidinylene, imidazolidinylene, pyrazolidylene, tetrahydrofuranylene, tetrahydropyranylene, tetrahydrothienylene, tetrahydrothiopyranylene, oxazolidinylene, thiazolidinylene, piperidinylene, piperazinylene, morpholinylene, thiomorpholinylene, azacycloheptanylene, azacyclooctylene, dioxacyclohexylene, diazacycloheptanylene, diazacyclooctylene, 6-azabicyclo[3.1.1]heptanylene, 2,5-diazabicyclo[2.2.1]heptanylene, 3,6-diazabicyclo[3.1.1]heptanylene, 3-azabicyclo[3.2.1]octanylene, 3,8-diazabicyclo[3.2.1]octanylene, 3,8-diazabicyclo[3.2.1]octanylene, 2,5-diazabicyclo[2.2.2]octanylene, 6-azabicyclo[3.1.1]heptanylene, 2,5-diazabicyclo[2.2.1]heptanylene, 3,6-diazabicyclo[3.1.1]heptanylene, 3-azabicyclo[3.2.1]octanylene, 3,8-diazabicyclo[3.2.1]octanylene, 3,8-diazabicyclo[3.2.1]octanylene, 2,5-diazabicyclo[2.2.2]octanylene, and azaspirocycloalkylene (e.g., 5-to 20-membered azaspirocycloalkylene, such as 3-azaspiro[5.5]undecanylene). In some embodiments, examples of the C₃₋₂₀ cycloalkylene include, but are not limited to, e.g., cyclopropylene, cyclobutylene, cyclopentylene, cyclopentenylene, cyclohexylene, cyclohexenylene, cycloheptylene, cyclooctylene, decalinylene, octahydropentalenylene, octahydro-1H-indenylene, 2,3-dihydro-1H-indenylene, spiro-cycloalkylene (e.g., C₅₋₁₅ spiro-cycloalkylene), adamantanylene, noradamantanylene, and norbornylene. In some embodiments, examples of the 5-to 20-membered arylene include, but are not limited to, e.g., phenylene or naphthylene. In some embodiments, the 5-to 20-membered heteroarylene is a 5-to 20-membered (e.g., 5-to 15-membered, 5-to 12-membered, 5-to 9-membered, 5-to 8-membered, 5-to 7-membered, or 5-to 6-membered) heteroarylene containing one nitrogen atom and optionally containing a heteroatom(s) selected from nitrogen, oxygen, and sulfur. In some embodiments, examples of the 5-to 20-membered heteroarylene include, but are not limited to, e.g., oxazolylene, isoxazolylene, oxadiazolylene, thienylene, thiazolylene, isothiazolylene, thiadiazolylene, pyrrolylene, imidazolylene, pyrazolylene, triazolylene, pyridylene, pyrimidinylene, pyridazinylene, pyrazinylene, indolylene, isoindolylene, benzofuranylene, isobenzofuranylene, benzothienylene, indazolylene, benzimidazolylene, benzoxazolylene, benzisoxazolylene, benzothiazolylene, benzisothiazolylene, benzotriazolylene, benzo[2,1,3]oxadiazolylene, benzo[2,1,3]thiadiazolylene, benzo[1,2,3]thiadiazolylene, quinolinylene, isoquinolinylene, naphthyridinylene, cinnolinylene, quinazolinylene, quinoxalinylene, phthalazinylene, pyrazolo[1,5-a]pyridylene, pyrazolo[1,5-a]pyrimidinylene, imidazo[1,2-a]pyridylene, 1H-pyrrolo[3,2-b]pyridylene, 1H-pyrrolo[2,3-b]pyridylene, pyrrolo[2,1-b]thiazolylene, and imidazo[2,1-b]thiazolylene. Each ring A² is independently optionally substituted with a2 R^{y2} substituents, and each R^{y2} independently represents deuterium, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), C₁₋₆ alkoxy (e.g., C₁₋₄ alkoxy, such as methoxy, ethoxy, propoxy, isopropoxy, n-butoxy, sec-butoxy, or tert-butoxy), halogen (e.g., fluorine, chlorine, bromine, or iodine), amino, hydroxy, mercapto, cyano, carboxyl, C₂₋₆ alkynyl, or C₂₋₆ alkenyl. a2 represents an integer of 0-20, e.g., an integer of 0, 1, 2, 3, 4, 5, 6, 7, 8, 9, 10, 11, 12, 13, 14, 15, or 20. The number of substituents is not theoretically limited in any way, or is automatically limited by the size of the building units.

In some embodiments, y2 of the compound of Formula (I) represents an integer of 0.

In some embodiments, y2 of the compound of Formula (I) represents an integer of 1.

In some embodiments, each ring A² of the compound of Formula (I) is the same or different and independently represents the following optionally substituted groups:

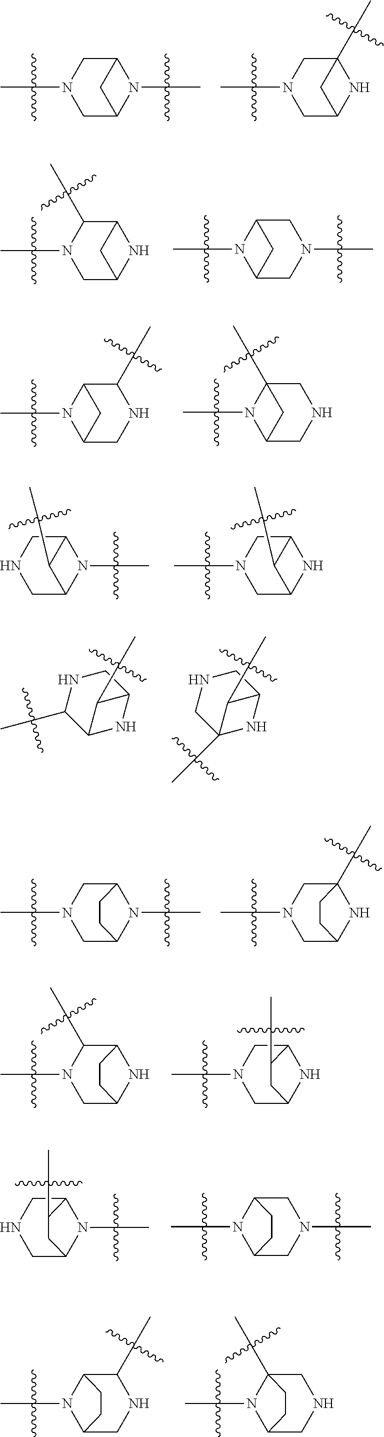

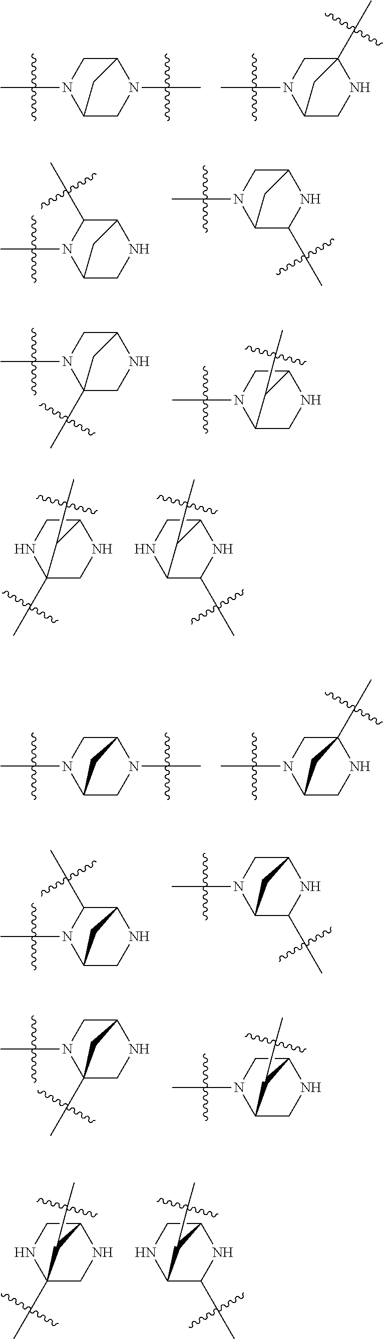

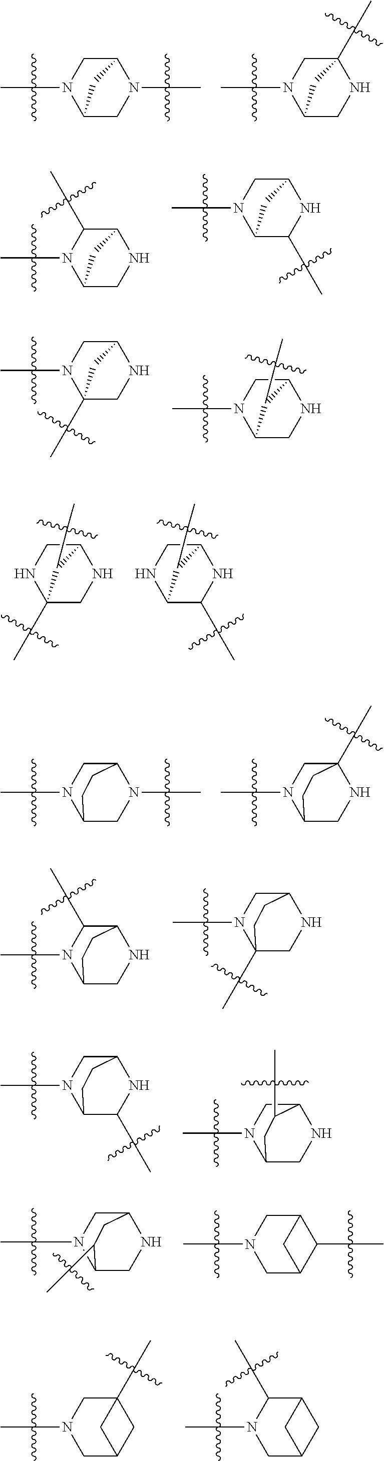

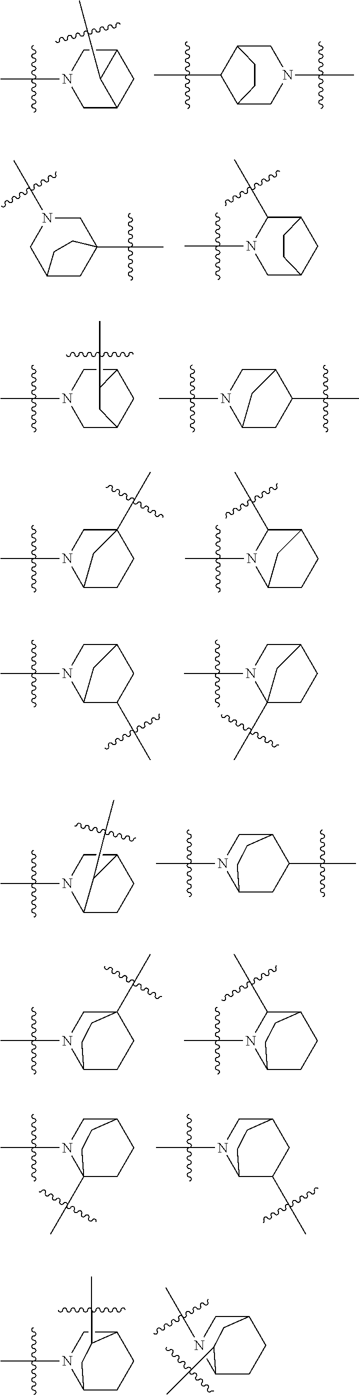

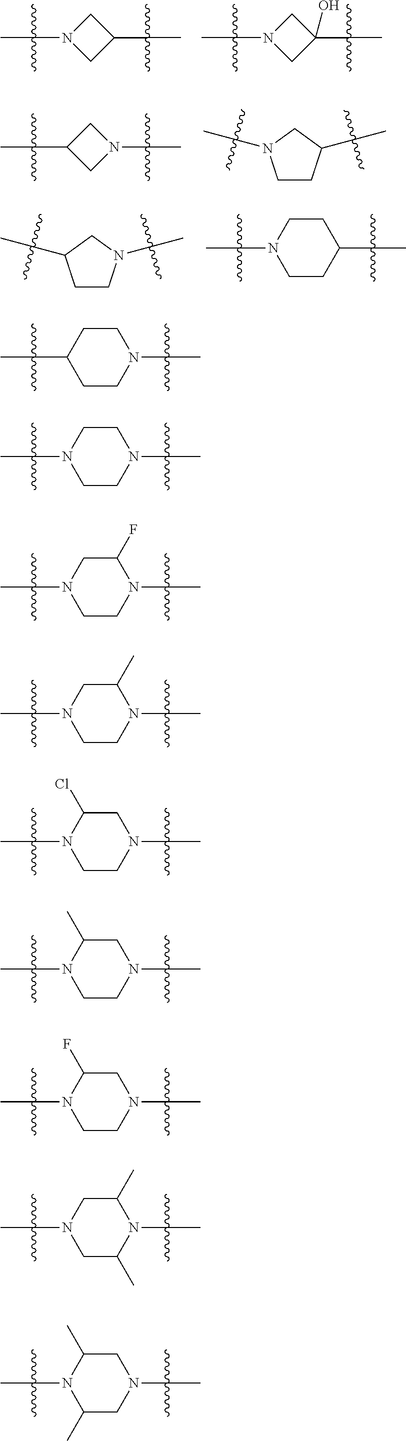

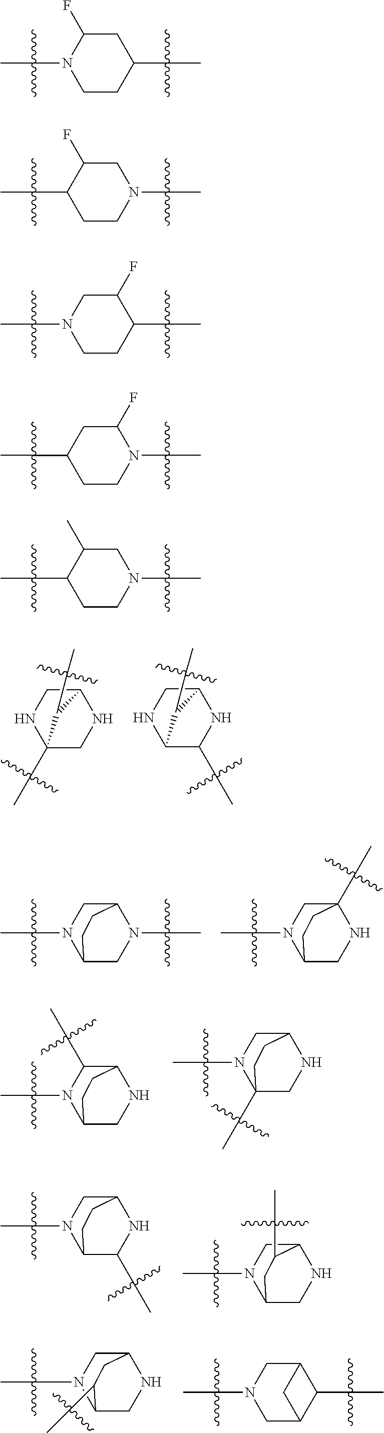

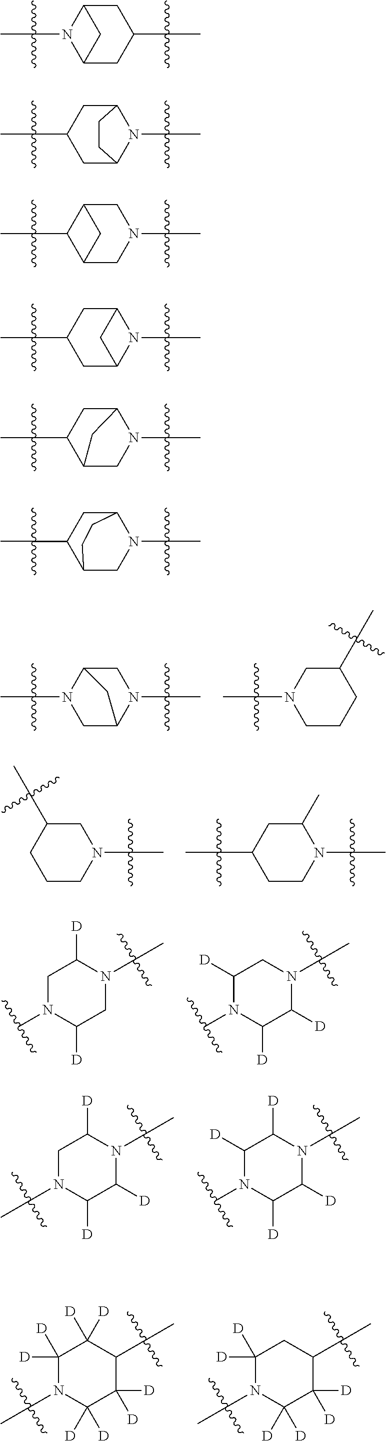

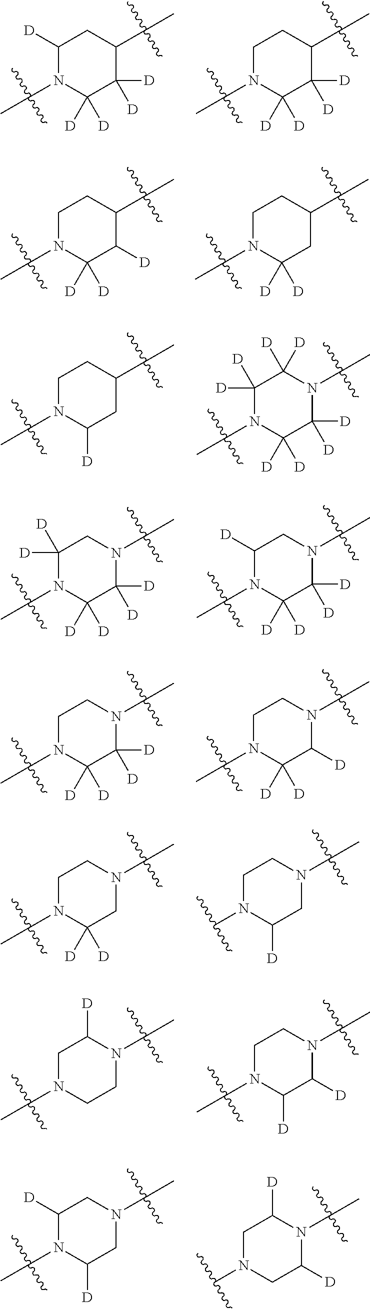

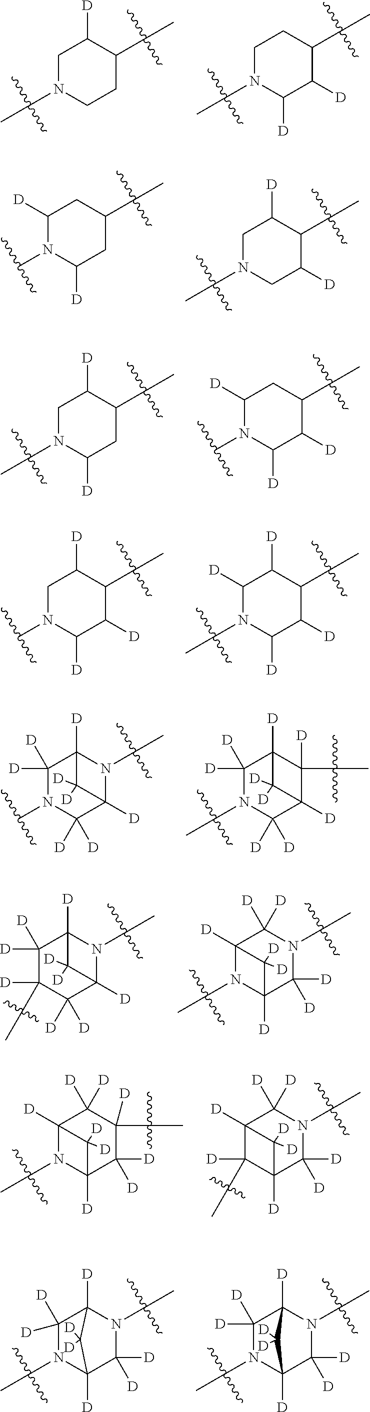

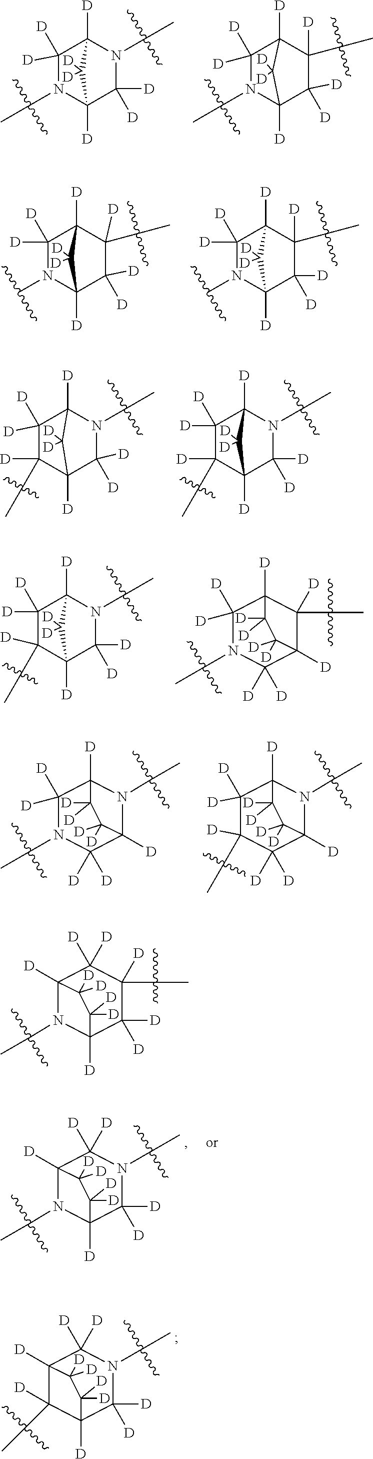

each ring A² is independently optionally substituted with a2 R^{y2} groups, wherein each R^{y2} represents independently deuterium, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), C₁₋₆ alkoxy (e.g., C₁₋₄ alkoxy, such as methoxy, ethoxy, propoxy, isopropoxy, n-butoxy, sec-butoxy, or tert-butoxy), halogen (e.g., fluorine, chlorine, bromine, or iodine), amino, hydroxy, mercapto, cyano, carboxyl, C₂₋₆ alkynyl (e.g., C₂₋₄ alkynyl, such as ethynyl, propynyl or butynyl), or C₂₋₆ alkenyl (e.g., C₂₋₄ alkenyl, such as vinyl, propenyl, or butenyl). a2 represents an integer of 0-20, such as an integer of 0, 1, 2, 3, 4, 5, 6, 7, 8, 9, 10, 11, 12, 13, 14, 15, or 20. The number of substituents is not theoretically limited in any way, or is automatically limited by the size of the building units.

In some embodiments, R_{w} of the compound of Formula (I) represents:

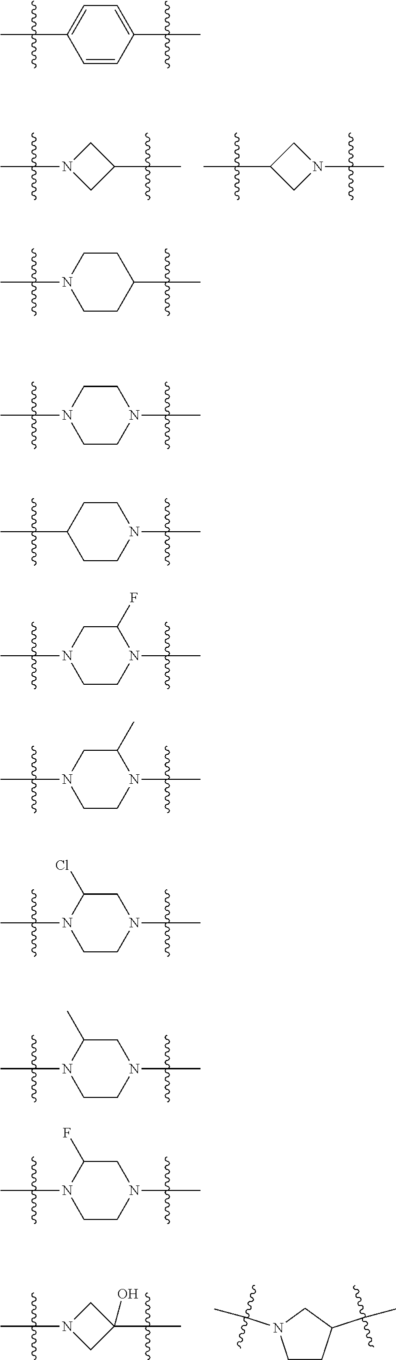

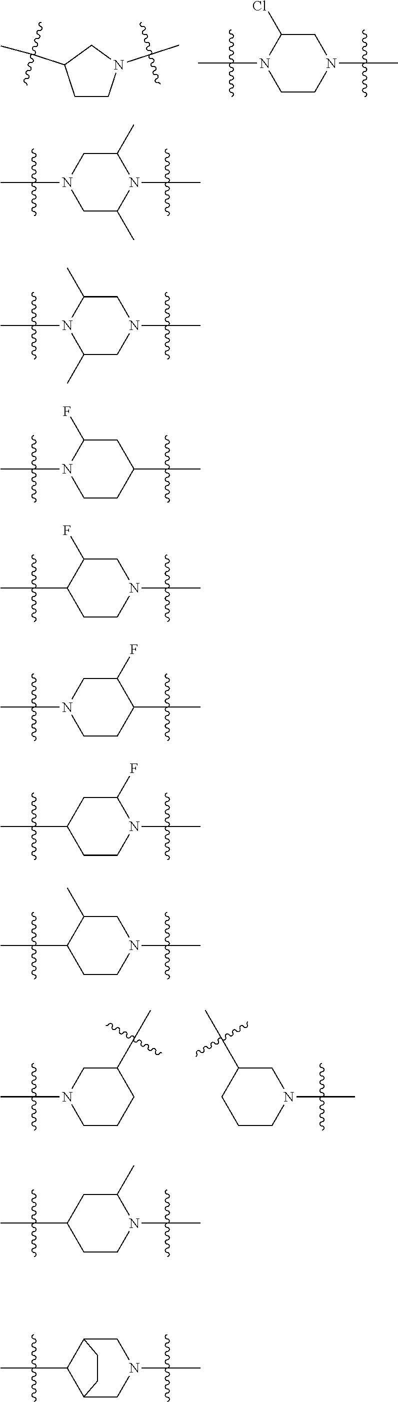

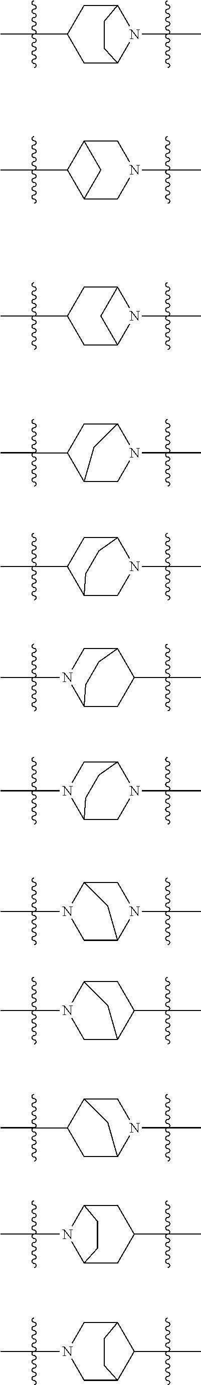

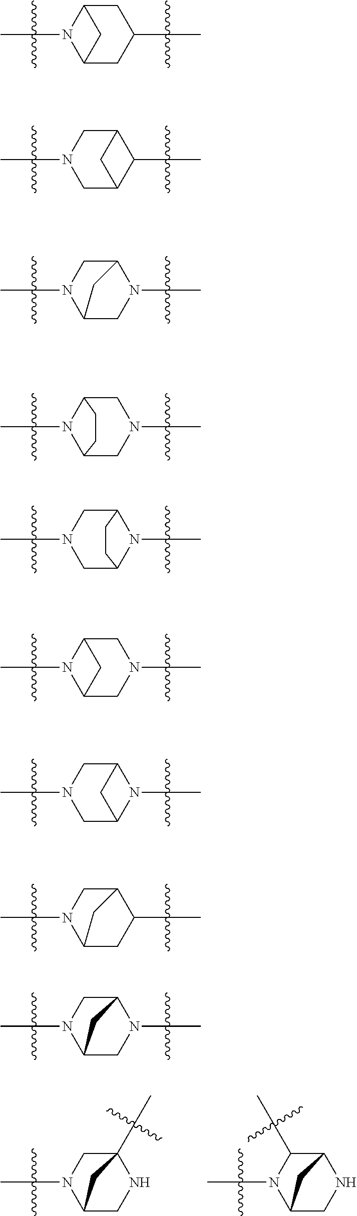

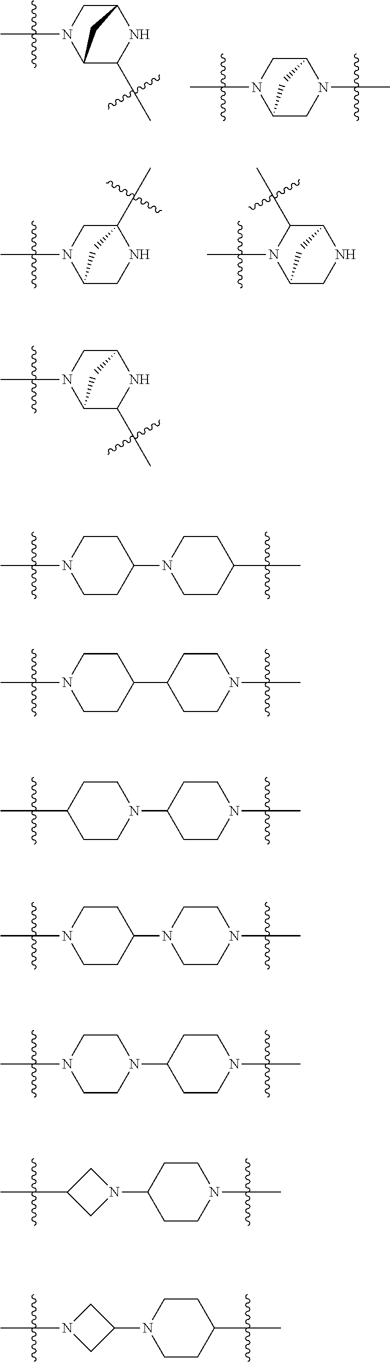

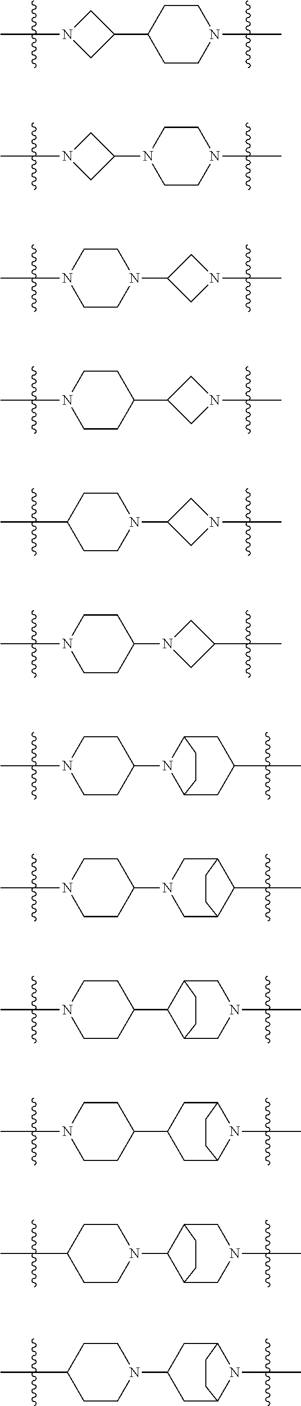

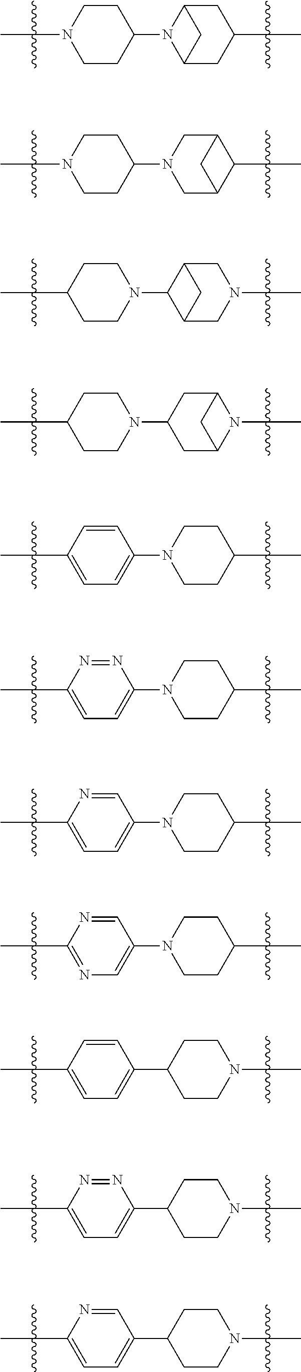

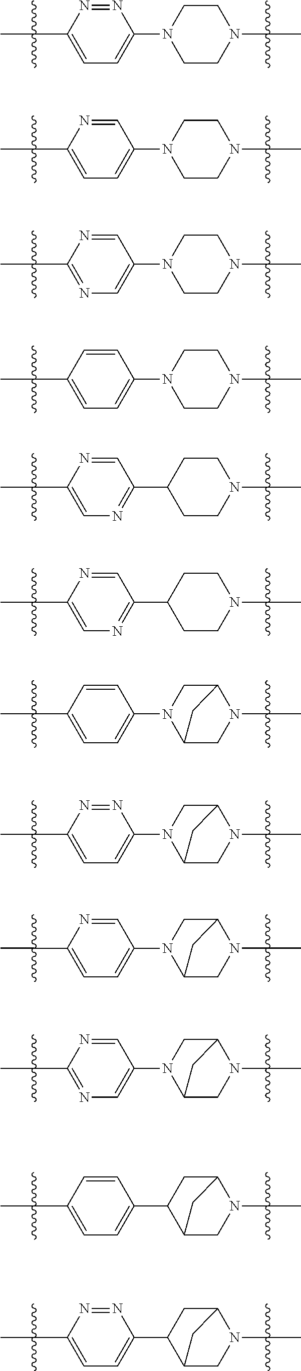

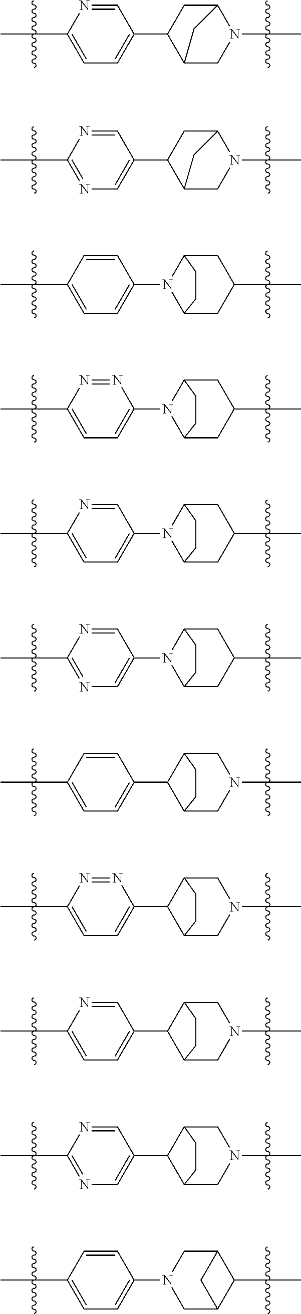

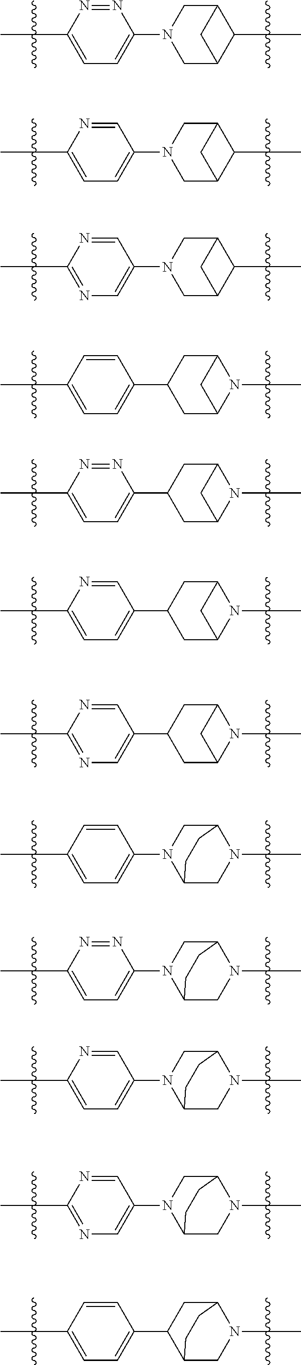

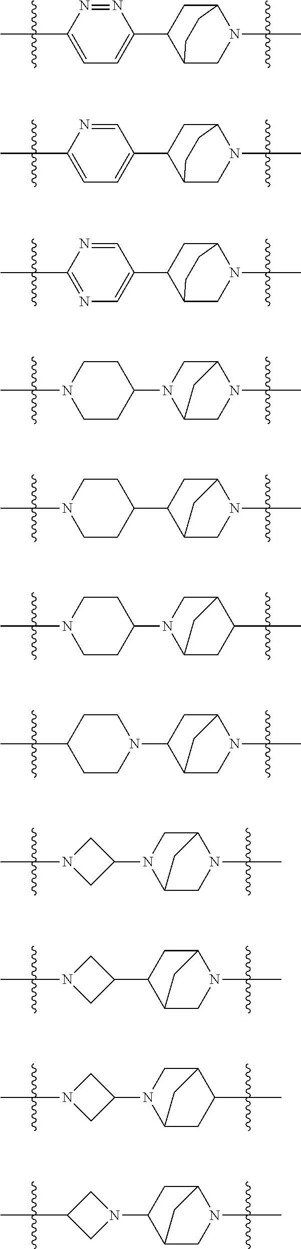

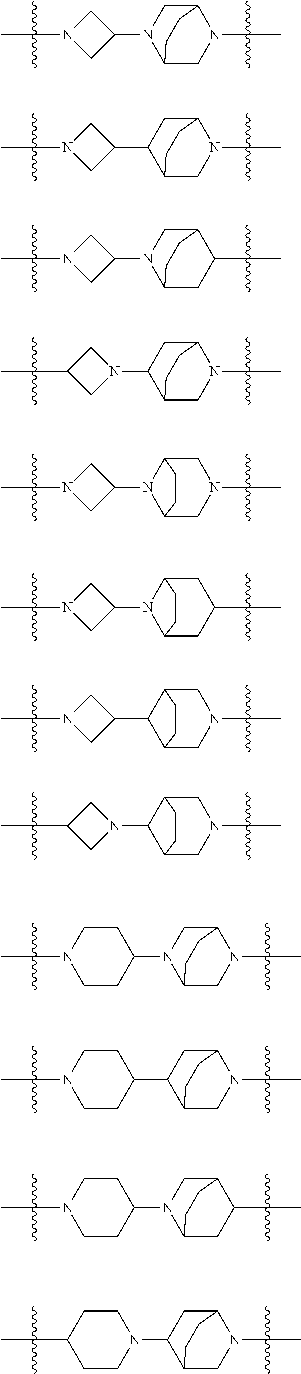

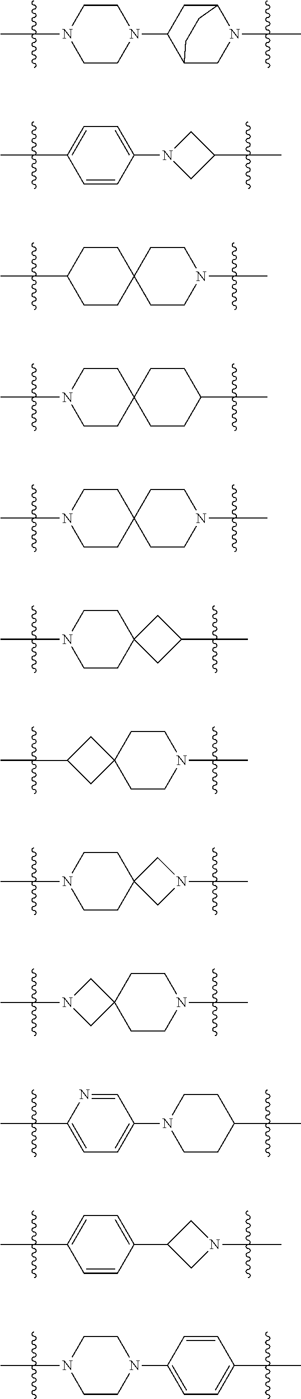

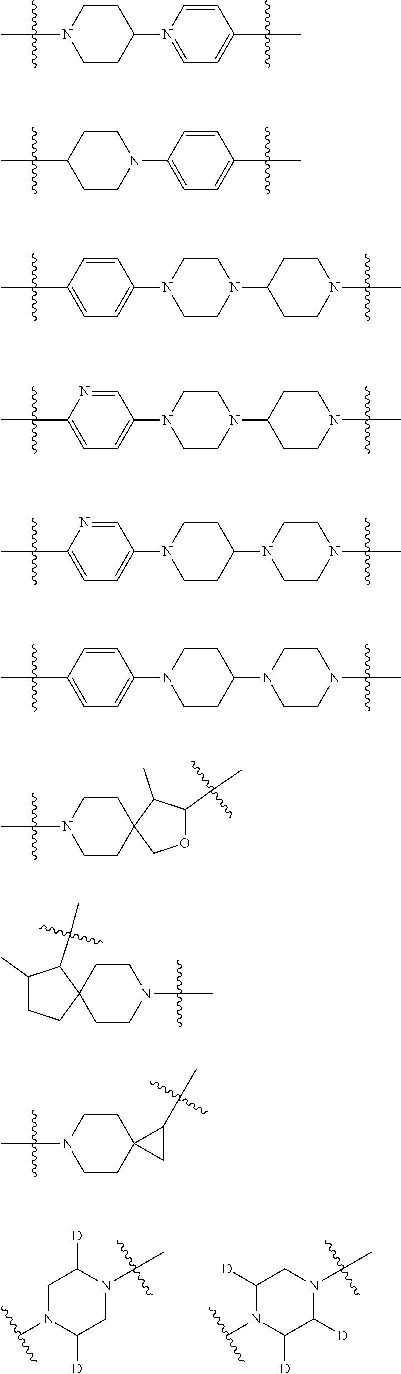

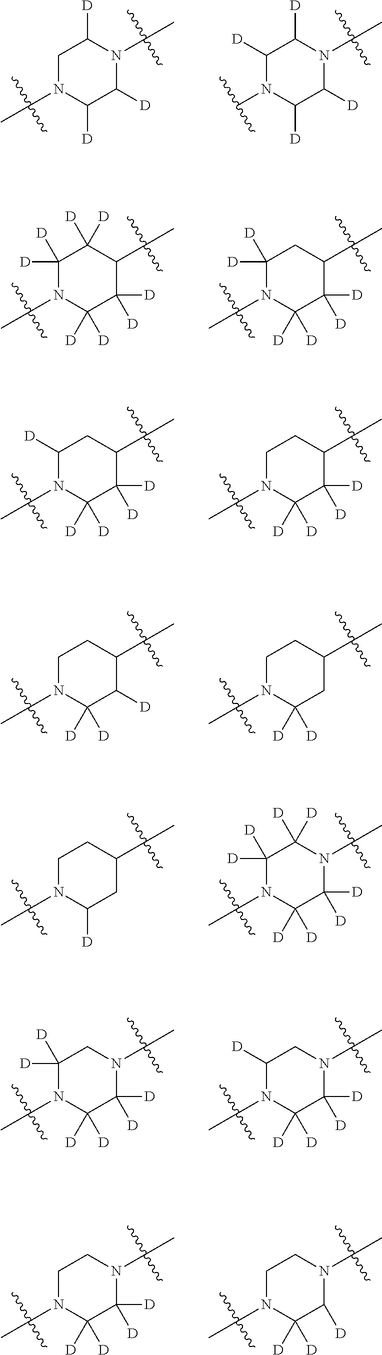

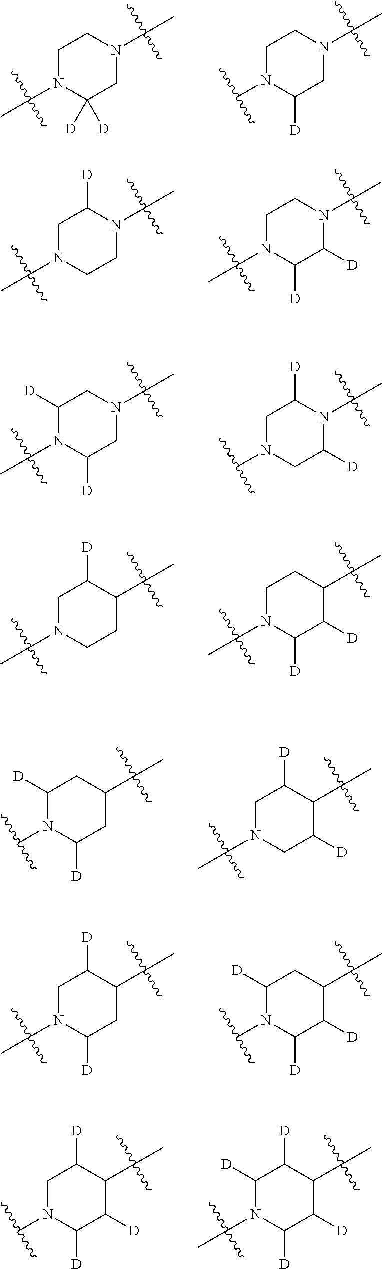

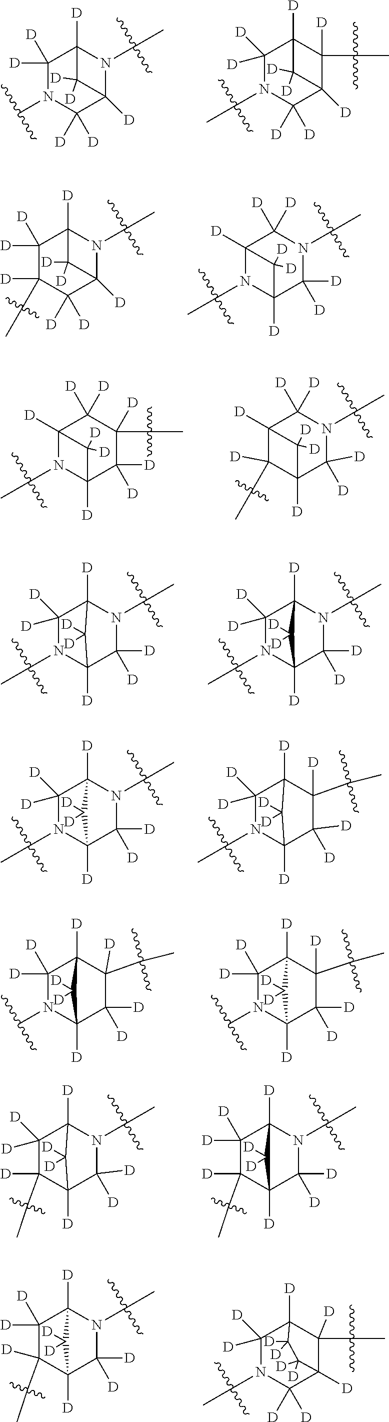

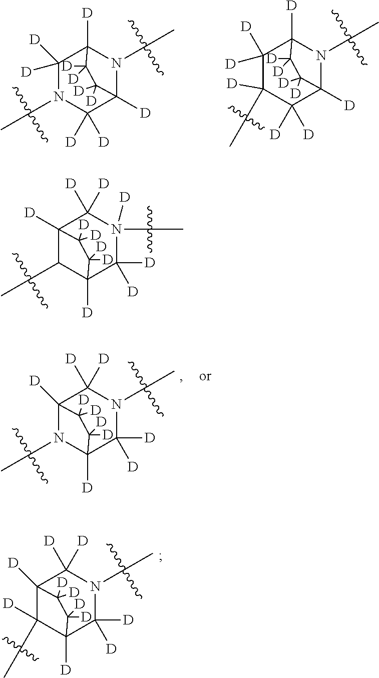

wherein the groups are optionally substituted with a substituent selected from the group consisting of deuterium, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), C₁₋₆ alkoxy (e.g., C₁₋₄ alkoxy, such as methoxy, ethoxy, propoxy, isopropoxy, n-butoxy, sec-butoxy, or tert-butoxy), halogen (e.g., fluorine, chlorine, bromine, or iodine), amino, hydroxy, mercapto, cyano, carboxyl, C₂₋₆ alkynyl (e.g., C₂₋₄ alkynyl, such as ethynyl, propynyl or butynyl), or C₂₋₆ alkenyl (e.g., C₂₋₄ alkenyl, such as vinyl, propenyl, or butenyl).

In some embodiments, ULM of the compound of Formula (I) represents a structure of Formula (ULM-1-1), Formula (ULM-1-2), Formula (ULM-1-3), or Formula (ULM-1-4):

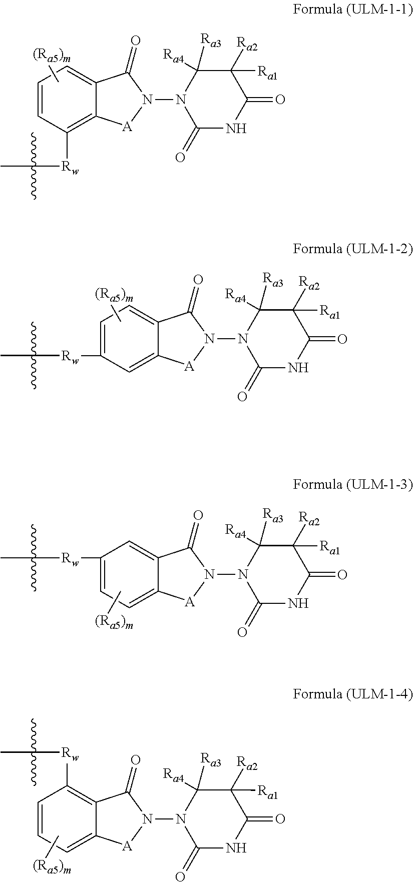

wherein A represents CO, CH₂, or CD₂;
Rₐ₁, Rₐ₂, Rₐ₃, and Rₐ₄ are the same or different and independently represent hydrogen, deuterium, halogen, C₁₋₆ alkyl, halogenated C₁₋₆ alkyl, deuterated C₁₋₆ alkyl, C₁₋₆ alkoxy, deuterated C₁₋₆ alkoxy, or halogenated C₁₋₆ alkoxy;
(Rₐ₅)ₘ indicates that the isoindoline ring to which it is attached is optionally substituted with m Ras, with each Ras being the same or different and each independently representing halogen, hydroxy, mercapto, nitro, amino, cyano, C₁₋₆ alkyl, halogenated C₁₋₆ alkyl, deuterated C₁₋₆ alkyl, C₁₋₆ alkoxy, deuterated C₁₋₆ alkoxy, halogenated C₁₋₆ alkoxy, C₂₋₆ alkenyl or C₂₋₆ alkynyl;
m represents an integer of 0, 1, 2, 3, or 4;
R_{w} represents O, S, S(O), S(O)₂, alkenylene, alkynylene, or N(Rₐ₆), wherein Rₐ₆ represents H or C₁₋₃ alkyl, or
R_{w} represents a bond, or
R_{w} represents:

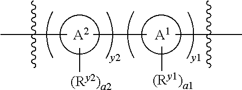

wherein each ring A¹ is the same or different and independently represents nitrogen-containing heterocyclylene (e.g., 4-to 20-membered nitrogen-containing heterocyclylene), arylene (e.g., C₅₋₂₀ arylene, C₆₋₁₅ arylene), or heteroarylene (e.g., 5-to 20-membered heteroarylene), y1 represents an integer of 1 or 2, and (R_{y1})ₐ₁ indicates that each ring A¹ is independently optionally substituted with a1 R_{y1} substituents, with each R^{y1} independently representing deuterium, C₁₋₆ alkyl, deuterated C₁₋₆ alkyl, halogenated C₁₋₆ alkyl, C₁₋₆ alkoxy, halogen, amino, hydroxy, mercapto, cyano, carboxyl, C₂₋₆ alkynyl, or C₂₋₆ alkenyl, and a1 represents an integer of 0-20 (e.g., an integer of 0, 1, 2, 3, 4, 5, 6, 7, 8, 9, 10, 11, 12, 13, 14, 15, or 20. The number of substituents is not theoretically limited in any way, or is automatically limited by the size of the building units);
each ring A² is the same or different and independently represents heterocyclylene (e.g., 4-to 20-membered heterocyclylene), cycloalkylene (e.g., C₃₋₂₀ cycloalkylene), arylene (e.g., C₅₋₂₀ arylene, or C₆₋₁₅ arylene), or heteroarylene (e.g., 5-to 20-membered heteroarylene), y2 represents an integer of 0 or 1, and (R^{y2})ₐ₂ indicates that each ring A² is independently optionally substituted with a2 R^{y2} substituents, with each R^{y2} independently representing deuterium, C₁₋₆ alkyl, deuterated C₁₋₆ alkyl, halogenated C₁₋₆ alkyl, C₁₋₆ alkoxy, halogen, amino, hydroxy, mercapto, cyano, carboxyl, C₂₋₆ alkynyl, or C₂₋₆ alkenyl, and a2 represents an integer of 0-20 (e.g., an integer of 0, 1, 2, 3, 4, 5, 6, 7, 8, 9, 10, 11, 12, 13, 14, 15, or 20. The number of substituents is not theoretically limited in any way, or is automatically limited by the size of the building units).

In some embodiments, ULM of the compound of Formula (I) represents a structure of the following Formulae:

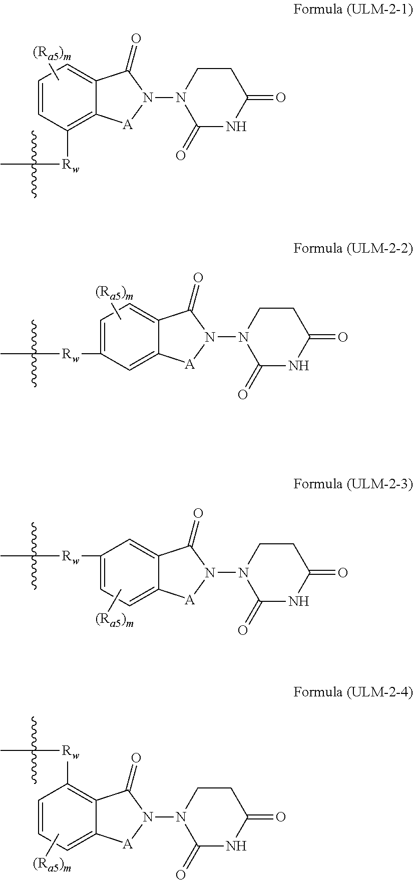

wherein the A, (Rₐ₅)ₘ, and R_{w} are as defined herein.

In some embodiments, LIN of the compound of Formula (I) represents:

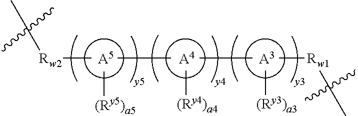

wherein R_{w2} is linked to PBM, and R_{w1} is linked to R_{w};
R_{w2} represents a bond, C(O), C(O)NH, or NHC(O);
each ring A³ is the same or different and independently represents heterocyclylene (e.g., 4-to 20-membered heterocyclylene), cycloalkylene (e.g., C₃₋₂₀cycloalkylene), arylene (e.g., C₅₋₂₀ arylene), or heteroarylene (e.g., 5-to 20-membered heteroarylene), y3 represents an integer of 0, 1, 2, or 3, and (R^{y3})ₐ₃ indicates that each ring A³ is independently optionally substituted with a3 R^{y3} substituents, with each R^{y3} independently representing deuterium, C₁₋₆ alkyl, deuterated C₁₋₆ alkyl, halogenated C₁₋₆ alkyl, C₁₋₆ alkoxy, halogen, amino, hydroxy, mercapto, cyano, carboxyl, C₂₋₆ alkynyl, or C₂₋₆ alkenyl, and a3 represents an integer of 0-10;
each ring A⁴ is the same or different and independently represents heterocyclylene (e.g., 4-to 20-membered heterocyclylene), cycloalkylene (e.g., C₃₋₂₀cycloalkylene), arylene (e.g., C₅₋₂₀ arylene), or heteroarylene (e.g., 5-to 20-membered heteroarylene), y4 represents an integer of 0, 1, 2, or 3, and (R^{y4})ₐ₄ indicates that each ring A⁴ is independently optionally substituted with a4 R^{y4} substituents, with each R^{y4} independently representing deuterium, C₁₋₆ alkyl, deuterated C₁₋₆ alkyl, halogenated C₁₋₆ alkyl, C₁₋₆ alkoxy, halogen, amino, hydroxy, mercapto, cyano, carboxyl, C₂₋₆ alkynyl, or C₂₋₆ alkenyl, and a4 represents an integer of 0-10;
each ring A⁵ is the same or different and independently represents heterocyclylene (e.g., 4-to 20-membered heterocyclylene), cycloalkylene (e.g., C₃₋₂₀cycloalkylene), arylene (e.g., C₅₋₂₀ arylene), or heteroarylene (e.g., 5-to 20-membered heteroarylene), y5 represents an integer of 0, 1, 2, or 3, and (R^{y5})ₐ₅ indicates that each ring A⁵ is independently optionally substituted with a5 R^{y5} substituents, with each R^{y5} independently representing deuterium, C₁₋₆ alkyl, deuterated C₁₋₆ alkyl, halogenated C₁₋₆ alkyl, C₁₋₆ alkoxy, halogen, amino, hydroxy, mercapto, cyano, carboxyl, C₂₋₆ alkynyl, or C₂₋₆ alkenyl, and a5 represents an integer of 0-10;
wherein R_{w1} represents a bond or optionally substituted linear or branched C₁₋₂₀ alkylene, wherein any one or more methylene groups in the main carbon chain skeleton of the linear or branched C₁₋₂₀ alkylene are optionally replaced by one or more groups selected from Rₐ, R_{b}, and any combination thereof; wherein each Rₐ is independently selected from the group consisting of: O, C(O), OC(O), S, S(O), S(O)₂, S(O)₂N(Rₐ₇), N(Rₐ₇)S(O)₂, C(O)N(Rₐ₇), N(Rₐ₇)C(O), N(Rₐ₇), and N(Rₐ₇)C(O)N(Rₐ₇), where each Raz is independently H or C₁₋₃ alkyl, and when any two or more methylene in the main carbon chain skeleton of the linear or branched C₁₋₂₀ alkylene are optionally replaced by two or more Rₐ groups, the Rₐ groups are not directly connected to each other; and wherein each R_{b} is independently selected from the group consisting of: optionally substituted cycloalkylene (e.g., optionally substituted C₃₋₂₀ cycloalkylene), optionally substituted arylene (e.g., optionally substituted C₅₋₂₀ arylene), optionally substituted heterocyclylene (e.g., optionally substituted 4-to 20-membered heterocyclylene), optionally substituted heteroarylene (e.g., optionally substituted 5-to 20-membered heteroarylene), alkynylene (e.g., C₂₋₆ alkynylene), alkenylene (e.g., C₂₋₆ alkenylene), and any combination thereof.

In some embodiments, R_{w2} of the compound of Formula (I) represents a bond.

In some embodiments, R_{w2} of the compound of Formula (I) represents C(O).

In some embodiments, R_{w2} of the compound of Formula (I) represents C(O)NH or NHC(O).

In some embodiments, each ring A³ of the compound of Formula (I) is the same or different and independently represents heterocyclylene (e.g., 4-to 20-membered heterocyclylene, such as 4-to 18-membered, 4-to 15-membered, 4-to 12-membered, 4-to 11-membered, 4-to 10-membered, 4-to 9-membered, 4-to 8-membered, 4-to 7-membered, 4-to 6-membered, 5-to 15-membered, or 5-to 9-memberedheterocyclylene), cycloalkylene (e.g., C₃₋₂₀ cycloalkylene), arylene (e.g., C₅₋₂₀ arylene, such as C₆₋₂₀ arylene, C₅₋₁₅ arylene, or C₆₋₁₅ arylene), or heteroarylene (e.g., 5-to 20-membered heteroarylene, such as 5-to 15-membered, 5-to 10-membered, 5-to 9-membered, 5-to 8-membered, 5-to 7-membered, or 5-to 6-membered heteroarylene). In some embodiments, 4-to 20-membered heterocyclylene is a 4-to 20-membered (e.g., 4-to 15-membered, 4-to 12-membered, 4-to 11-membered, 4-to 10-membered, 4-to 9-membered, 4-to 8-membered, 4-to 7-membered, 4-to 6-membered, 5-to 15-membered, or 5-to 9-membered) saturated or partially unsaturated (i.e., containing one or more double bonds, but not having a fully conjugated x-electron system) divalent monocyclic, bicyclic, tricyclic or polycyclic cyclic hydrocarbon group containing one or more (e.g., from 1 to 5, from 1 to 4, or from 1 to 3, or from 1 to 2, or 1) heteroatoms selected from the group consisting of oxygen, nitrogen, and sulfur. Examples of the 4-to 20-membered heterocyclylene include, but are not limited to, monocyclic heterocyclylene (e.g., 4-to 20-membered monocyclic heterocyclylene), bridged heterocyclylene (e.g., 5-to 20-membered bridged heterocyclylene, or 7-to 15-membered bridged heterocyclylene), fused heterocyclylene, and spiro-heterocyclylene (e.g., 5-to 20-membered spiro-heterocyclylene), e.g., azetidinylene, oxetanylene, pyrrolidinylene, imidazolidinylene, pyrazolidylene, tetrahydrofuranylene, tetrahydropyranylene, tetrahydrothienylene, tetrahydrothiopyranylene, oxazolidinylene, thiazolidinylene, piperidinylene, piperazinylene, morpholinylene, thiomorpholinylene, azacycloheptanylene, azacyclooctylene, dioxacyclohexylene, diazacycloheptanylene, diazacyclooctylene, 6-azabicyclo[3.1.1]heptanylene, 2,5-diazabicyclo[2.2.1]heptanylene, 3,6-diazabicyclo[3.1.1]heptanylene, 3-azabicyclo[3.2.1]octanylene, 3,8-diazabicyclo[3.2.1]octanylene, 3,8-diazabicyclo[3.2.1]octanylene, 2,5-diazabicyclo[2.2.2]octanylene, and azaspirocycloalkylene (e.g., 5-to 20-membered azaspirocycloalkylene, such as 3-azaspiro[5.5]undecanylene). In some embodiments, examples of the C₃₋₂₀ cycloalkylene include, but are not limited to, e.g., cyclopropylene, cyclobutylene, cyclopentylene, cyclopentenylene, cyclohexylene, cyclohexenylene, cycloheptylene, cyclooctylene, decalinylene, octahydropentalenylene, octahydro-1H-indenylene, 2,3-dihydro-1H-indenylene, spiro-cycloalkylene (e.g., C₅₋₁₅ spiro-cycloalkylene), adamantanylene, noradamantanylene, and norbornylene. In some embodiments, examples of the 5-to 20-membered arylene include, but are not limited to, e.g., phenylene or naphthylene. In some embodiments, 5-to 20-membered heteroarylene is a 5-to 20-membered (e.g., 5-to 15-membered, 5-to 12-membered, 5-to 9-membered, 5-to 8-membered, 5-to 7-membered, or 5-to 6-membered) heteroarylene containing one nitrogen atom and optionally containing a heteroatom(s) selected from nitrogen, oxygen, and sulfur. In some embodiments, examples of the 5-to 20-membered heteroarylene include, but are not limited to, e.g., oxazolylene, isoxazolylene, oxadiazolylene, thienylene, thiazolylene, isothiazolylene, thiadiazolylene, pyrrolylene, imidazolylene, pyrazolylene, triazolylene, pyridylene, pyrimidinylene, pyridazinylene, pyrazinylene, indolylene, isoindolylene, benzofuranylene, isobenzofuranylene, benzothienylene, indazolylene, benzimidazolylene, benzoxazolylene, benzisoxazolylene, benzothiazolylene, benzisothiazolylene, benzotriazolylene, benzo[2,1,3]oxadiazolylene, benzo[2,1,3]thiadiazolylene, benzo[1,2,3]thiadiazolylene, quinolinylene, isoquinolinylene, naphthyridinylene, cinnolinylene, quinazolinylene, quinoxalinylene, phthalazinylene, pyrazolo[1,5-a]pyridylene, pyrazolo[1,5-a]pyrimidinylene, imidazo[1,2-a]pyridylene, 1H-pyrrolo[3,2-b]pyridylene, 1H-pyrrolo[2,3-b]pyridylene, pyrrolo[2,1-b]thiazolylene, and imidazo[2,1-b]thiazolylene. Each ring A³ is independently optionally substituted with a3 R^{y3} substituents, and each R^{y3} independently represents deuterium, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), C₁₋₆ alkoxy (e.g., C₁₋₄ alkoxy, such as methoxy, ethoxy, propoxy, isopropoxy, n-butoxy, sec-butoxy, or tert-butoxy), halogen (e.g., fluorine, chlorine, bromine, or iodine), amino, hydroxy, mercapto, cyano, carboxyl, C₂₋₆ alkynyl, or C₂₋₆ alkenyl. a3 represents an integer of 0-10, e.g., an integer of 0, 1, 2, 3, 4, 5, 6, 7, 8, 9, or 10. The number of substituents is not theoretically limited in any way, or is automatically limited by the size of the building units.

In some embodiments, y3 of the compound of Formula (I) represents an integer of 0, 1, 2, or 3.

In some embodiments, each ring A³ of the compound of Formula (I) is the same or different and independently represents the following optionally substituted groups:

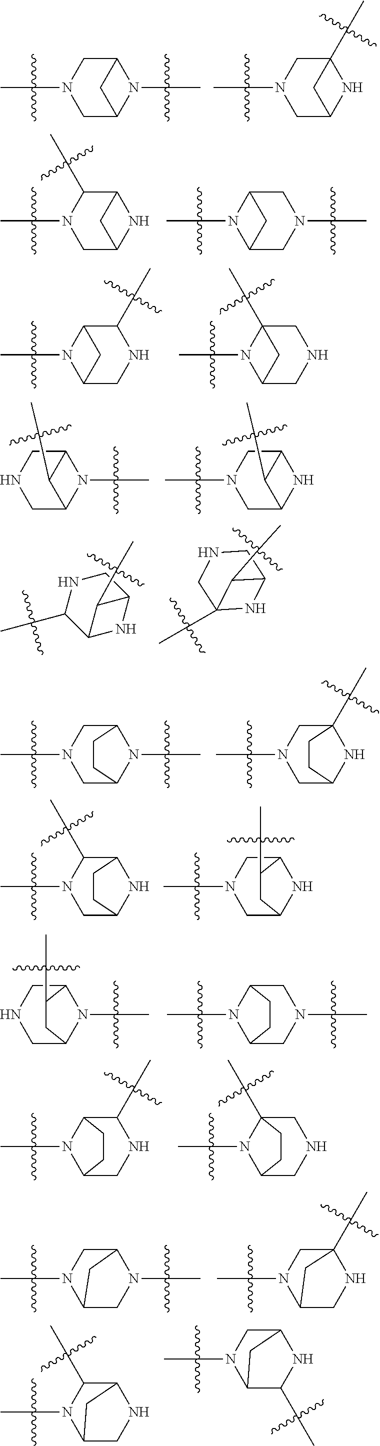

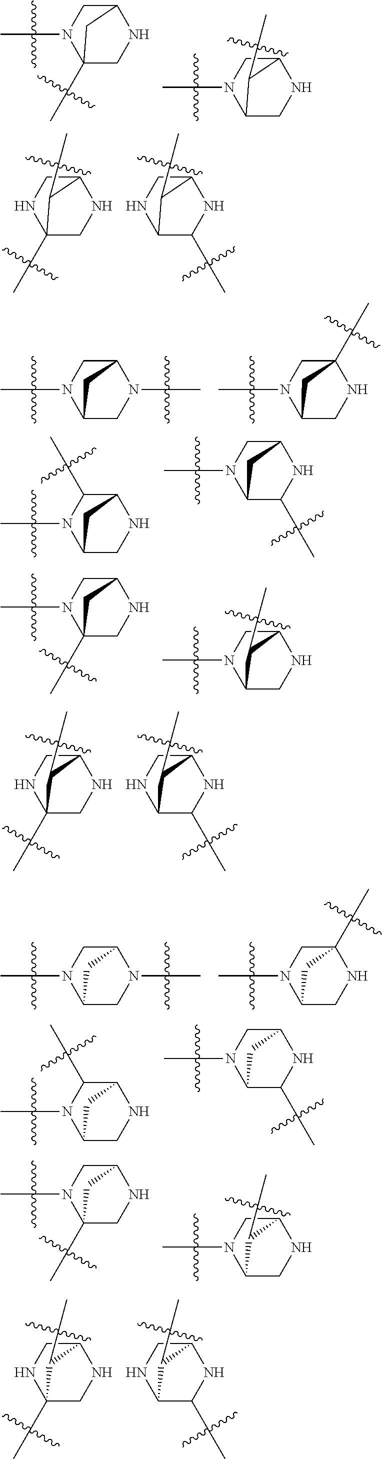

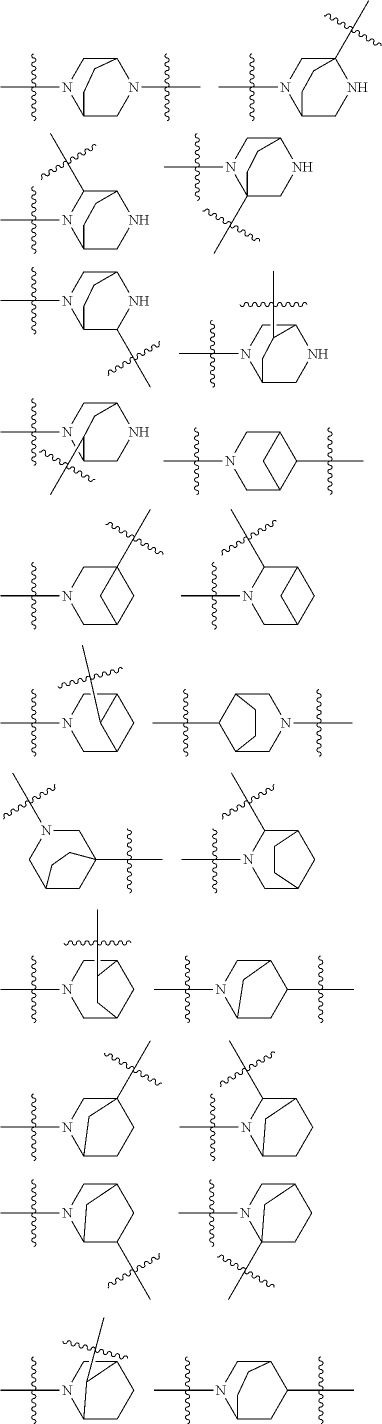

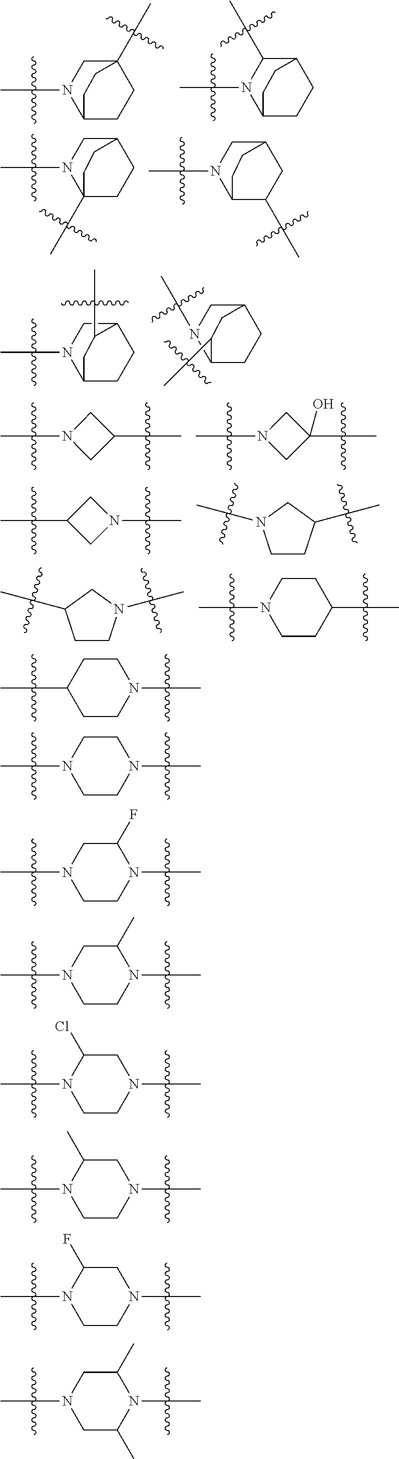

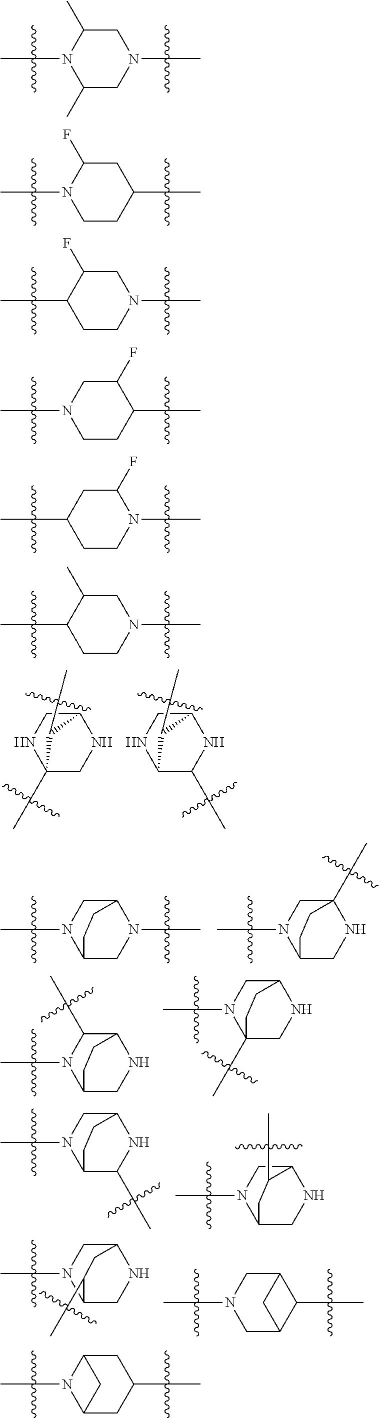

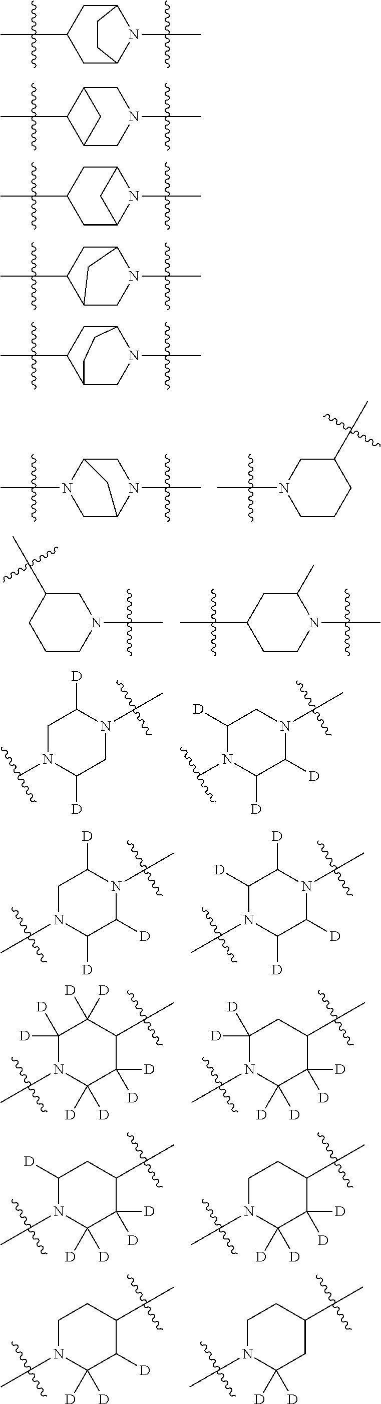

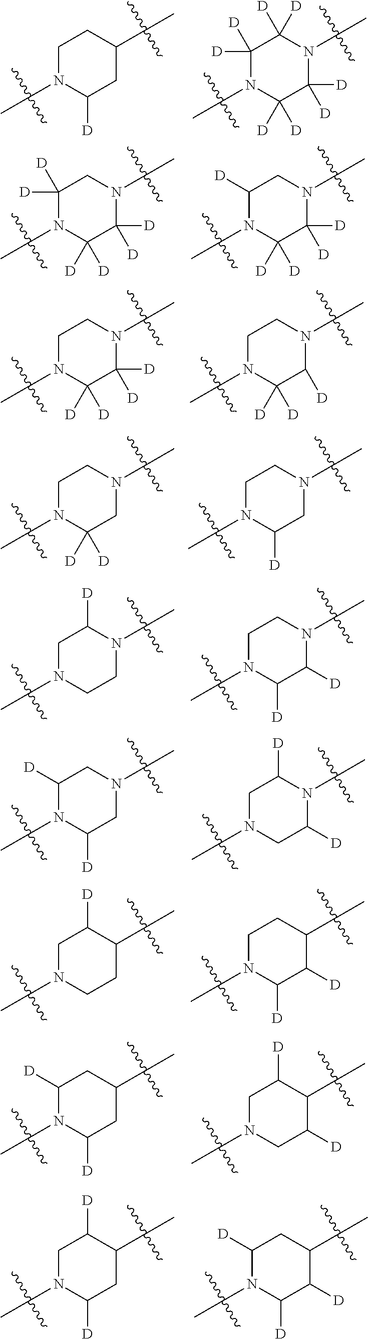

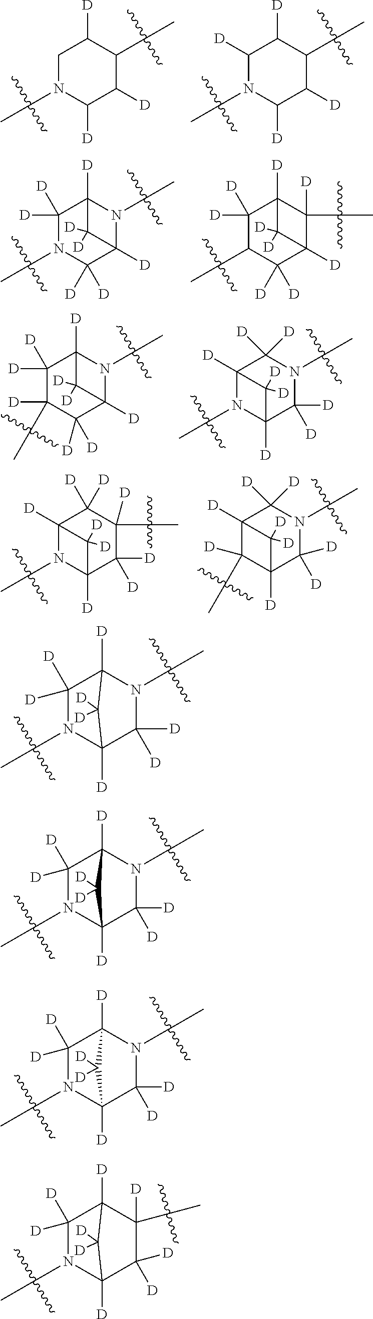

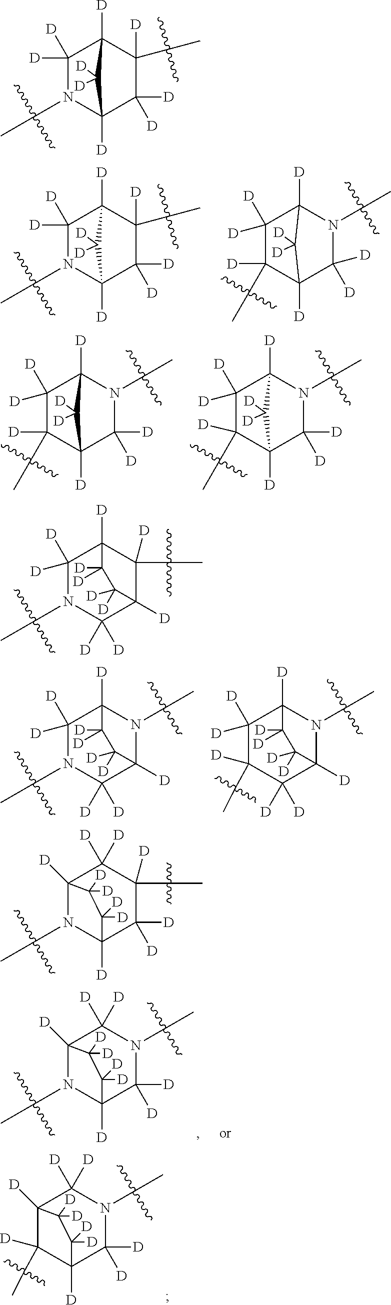

each ring A³ is independently optionally substituted with a3 R^{y3} substituents, and each R^{y3} independently represents deuterium, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), C₁₋₆ alkoxy (e.g., C₁₋₄ alkoxy, such as methoxy, ethoxy, propoxy, isopropoxy, n-butoxy, sec-butoxy, or tert-butoxy), halogen (e.g., fluorine, chlorine, bromine, or iodine), amino, hydroxy, mercapto, cyano, carboxyl, C₂₋₆ alkynyl (e.g., C₂₋₄ alkynyl, such as ethynyl, propynyl or butynyl), or C₂₋₆ alkenyl (e.g., C₂₋₄ alkenyl, such as vinyl, propenyl, or butenyl). a3 represents an integer of 0-10, e.g., an integer of 0, 1, 2, 3, 4, 5, 6, 7, 8, 9, or 10. The number of substituents is not theoretically limited in any way, or is automatically limited by the size of the building units.

In some embodiments, each ring A⁴ of the compound of Formula (I) is the same or different and independently represents heterocyclylene (e.g., 4-to 20-membered heterocyclylene, such as 4-to 18-membered, 4-to 15-membered, 4-to 12-membered, 4-to 11-membered, 4-to 10-membered, 4-to 9-membered, 4-to 8-membered, 4-to 7-membered, 4-to 6-membered, 5-to 15-membered, or 5-to 9-membered heterocyclylene), cycloalkylene (e.g., C₃₋₂₀ cycloalkylene), arylene (e.g., C₅₋₂₀ arylene, such as C₆₋₂₀ arylene, C₅₋₁₅ arylene, or C₆₋₁₅ arylene), or heteroarylene (e.g., 5-to 20-membered heteroarylene, such as 5-to 15-membered, 5-to 10-membered, 5-to 9-membered, 5-to 8-membered, 5-to 7-membered, or 5-to 6-membered heteroarylene). In some embodiments, the 4-to 20-membered heterocyclylene is a 4-to 20-membered (e.g., 4-to 15-membered, 4-to 12-membered, 4-to 11-membered, 4-to 10-membered, 4-to 9-membered, 4-to 8-membered, 4-to 7-membered, 4-to 6-membered, 5-to 15-membered, or 5-to 9-membered) saturated or partially unsaturated (i.e., containing one or more double bonds, but not having a fully conjugated x-electron system) divalent monocyclic, bicyclic, tricyclic or polycyclic cyclic hydrocarbon group containing one or more (e.g., from 1 to 5, from 1 to 4, or from 1 to 3, or from 1 to 2, or 1) heteroatoms selected from the group consisting of oxygen, nitrogen, and sulfur. Examples of the 4-to 20-membered heterocyclylene include, but are not limited to, monocyclic heterocyclylene (e.g., 4-to 20-membered monocyclic heterocyclylene), bridged heterocyclylene (e.g., 5-to 20-membered bridged heterocyclylene, or 7-to 15-membered bridged heterocyclylene), fused heterocyclylene, and spiro-heterocyclylene (e.g., 5-to 20-membered spiro-heterocyclylene), e.g., azetidinylene, oxetanylene, pyrrolidinylene, imidazolidinylene, pyrazolidylene, tetrahydrofuranylene, tetrahydropyranylene, tetrahydrothienylene, tetrahydrothiopyranylene, oxazolidinylene, thiazolidinylene, piperidinylene, piperazinylene, morpholinylene, thiomorpholinylene, azacycloheptanylene, azacyclooctylene, dioxacyclohexylene, diazacycloheptanylene, diazacyclooctylene, 6-azabicyclo[3.1.1]heptanylene, 2,5-diazabicyclo[2.2.1]heptanylene, 3,6-diazabicyclo[3.1.1]heptanylene, 3-azabicyclo[3.2.1]octanylene, 3,8-diazabicyclo[3.2.1]octanylene, 3,8-diazabicyclo[3.2.1]octanylene, 2,5-diazabicyclo[2.2.2]octanylene, and azaspirocycloalkylene (e.g., 5-to 20-membered azaspirocycloalkylene, such as 3-azaspiro[5.5]undecanylene). In some embodiments, examples of the C₃₋₂₀ cycloalkylene include, but are not limited to, e.g., cyclopropylene, cyclobutylene, cyclopentylene, cyclopentenylene, cyclohexylene, cyclohexenylene, cycloheptylene, cyclooctylene, decalinylene, octahydropentalenylene, octahydro-1H-indenylene, 2,3-dihydro-1H-indenylene, spiro-cycloalkylene (e.g., C₅₋₁₅ spiro-cycloalkylene), adamantanylene, noradamantanylene, and norbornylene. In some embodiments, examples of the 5-to 20-membered arylene include, but are not limited to, e.g., phenylene or naphthylene. In some embodiments, the 5-to 20-membered heteroarylene is a 5-to 20-membered (e.g., 5-to 15-membered, 5-to 12-membered, 5-to 9-membered, 5-to 8-membered, 5-to 7-membered, or 5-to 6-membered) heteroarylene containing one nitrogen atom and optionally containing a heteroatom(s) selected from nitrogen, oxygen, and sulfur. In some embodiments, examples of the 5-to 20-membered heteroarylene include, but are not limited to, e.g., oxazolylene, isoxazolylene, oxadiazolylene, thienylene, thiazolylene, isothiazolylene, thiadiazolylene, pyrrolylene, imidazolylene, pyrazolylene, triazolylene, pyridylene, pyrimidinylene, pyridazinylene, pyrazinylene, indolylene, isoindolylene, benzofuranylene, isobenzofuranylene, benzothienylene, indazolylene, benzimidazolylene, benzoxazolylene, benzisoxazolylene, benzothiazolylene, benzisothiazolylene, benzotriazolylene, benzo[2,1,3]oxadiazolylene, benzo[2,1,3]thiadiazolylene, benzo[1,2,3]thiadiazolylene, quinolinylene, isoquinolinylene, naphthyridinylene, cinnolinylene, quinazolinylene, quinoxalinylene, phthalazinylene, pyrazolo[1,5-a]pyridylene, pyrazolo[1,5-a]pyrimidinylene, imidazo[1,2-a]pyridylene, 1H-pyrrolo[3,2-b]pyridylene, 1H-pyrrolo[2,3-b]pyridylene, pyrrolo[2,1-b]thiazolylene, and imidazo[2,1-b]thiazolylene. Each ring A⁴ is independently optionally substituted with a4 R^{y4} substituents, and each R^{y4} independently represents deuterium, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), C₁₋₆ alkoxy (e.g., C₁₋₄ alkoxy, such as methoxy, ethoxy, propoxy, isopropoxy, n-butoxy, sec-butoxy, or tert-butoxy), halogen (e.g., fluorine, chlorine, bromine, or iodine), amino, hydroxy, mercapto, cyano, carboxyl, C₂₋₆ alkynyl, or C₂₋₆ alkenyl. a4 represents an integer of 0-10, e.g., an integer of 0, 1, 2, 3, 4, 5, 6, 7, 8, 9, or 10. The number of substituents is not theoretically limited in any way, or is automatically limited by the size of the building units.

In some embodiments, y4 of the compound of Formula (I) represents an integer of 0, 1, 2, or 3.

In some embodiments, each ring A⁴ of the compound of Formula (I) is the same or different and independently represents the following optionally substituted groups:

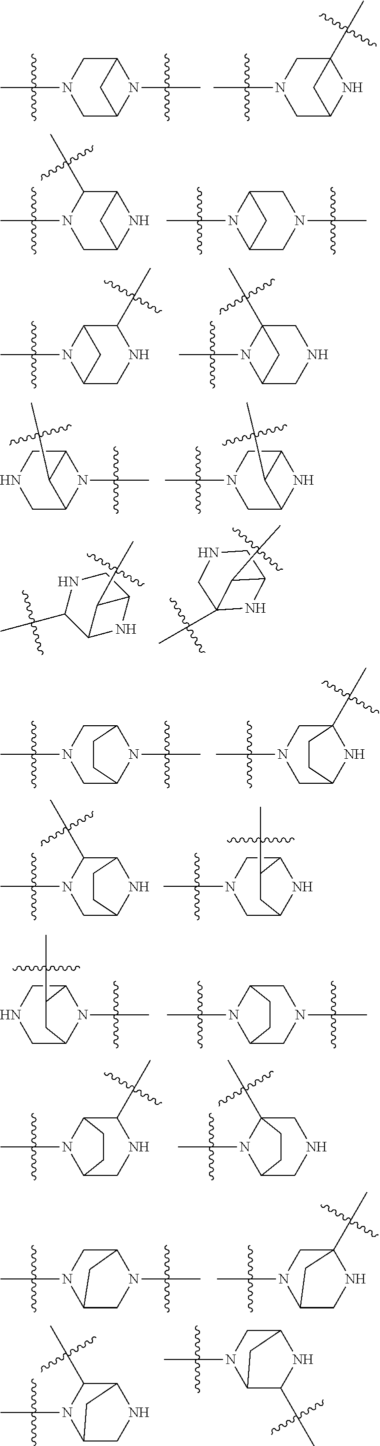

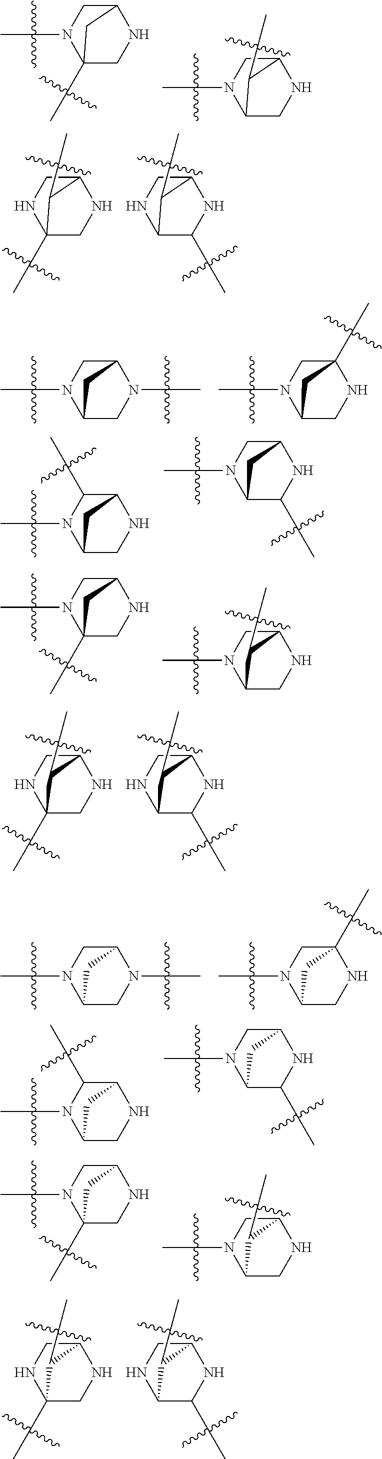

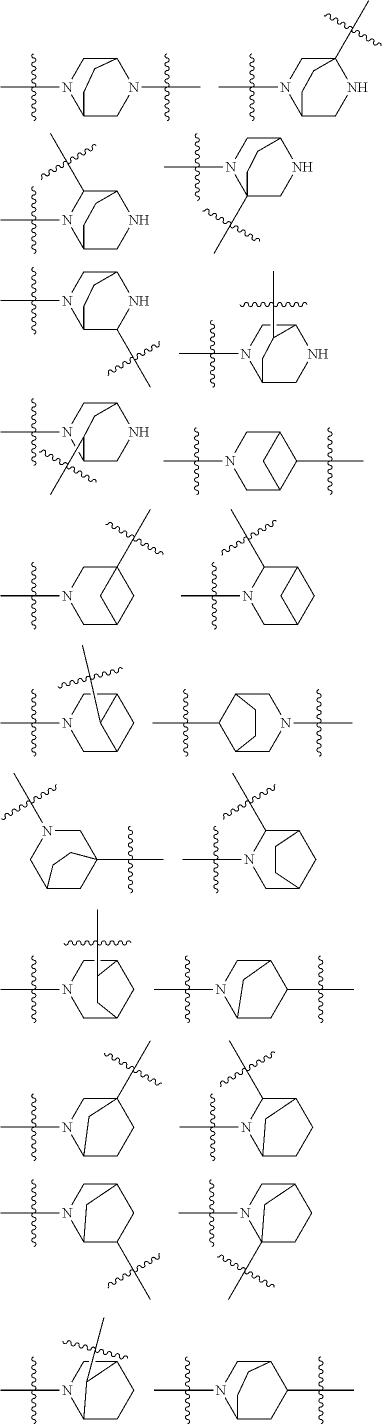

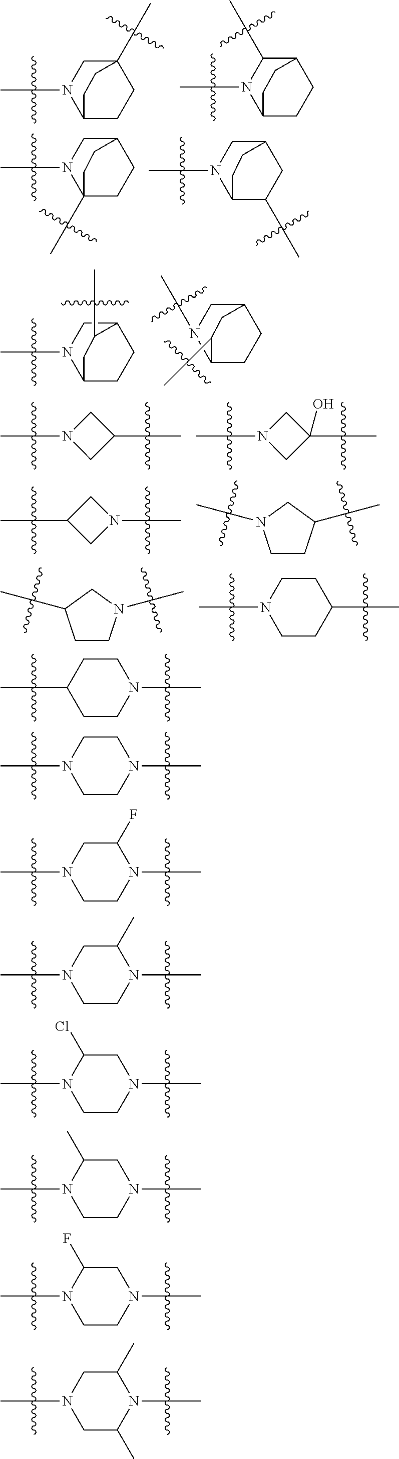

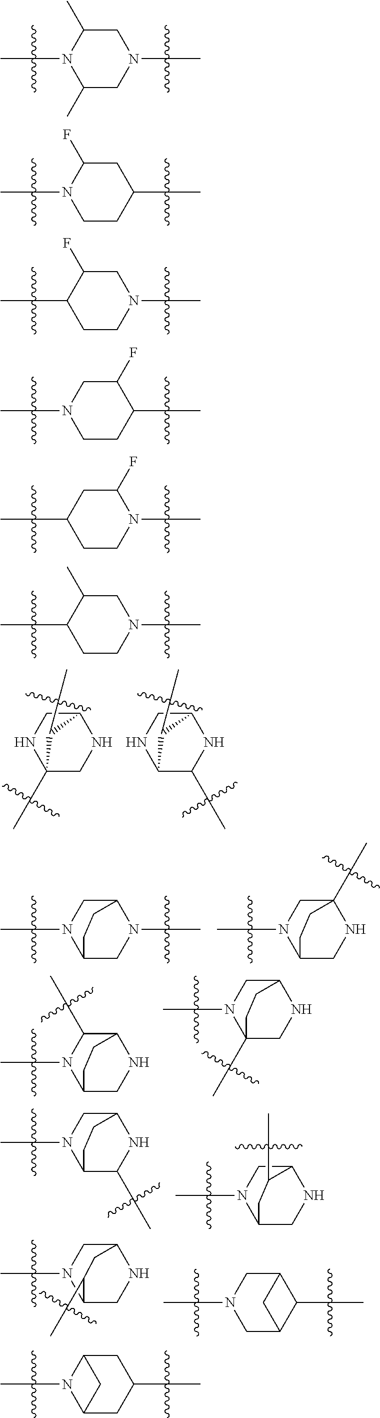

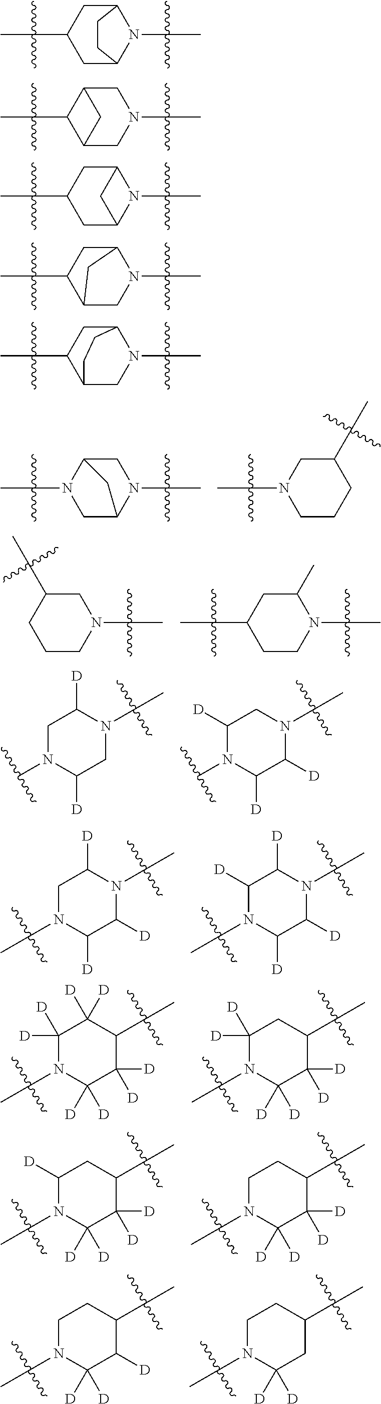

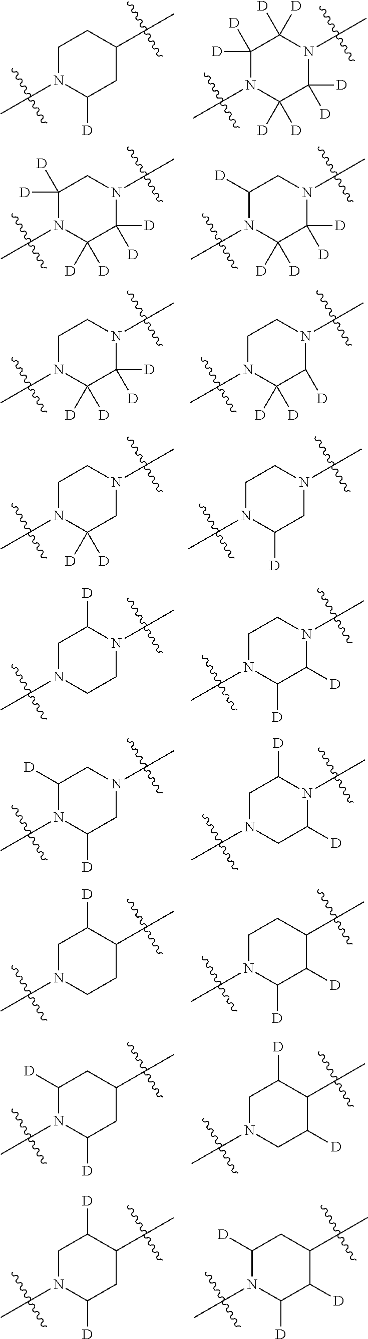

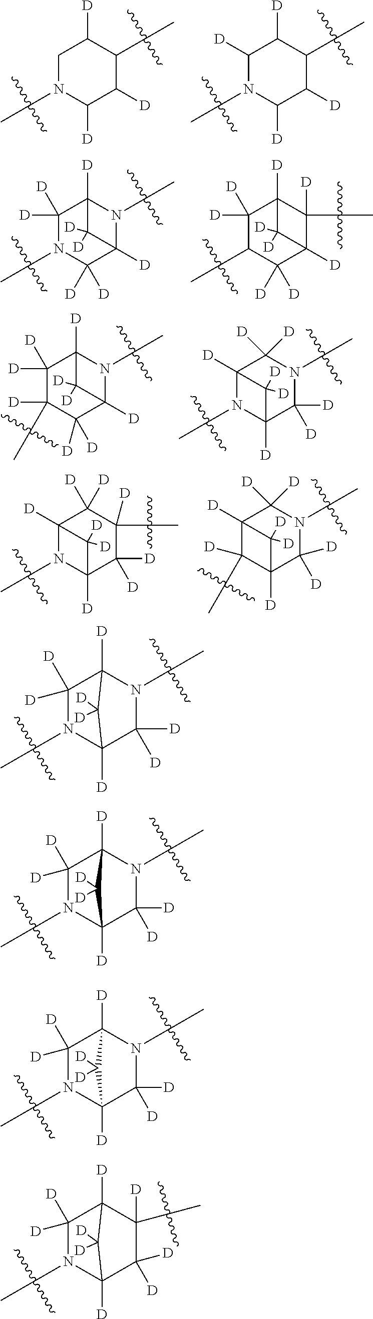

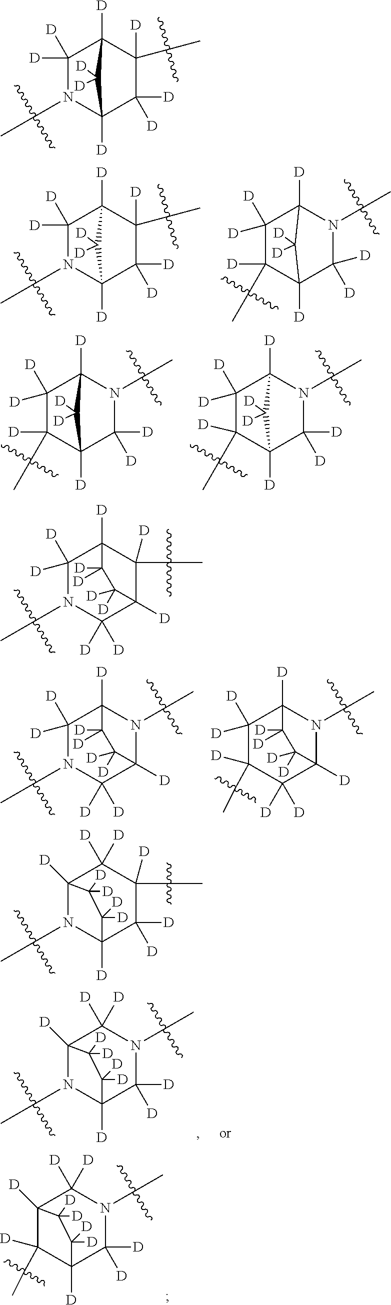

each ring A⁴ is independently optionally substituted with a4 R^{y4} groups, wherein each R^{y4} represents independently deuterium, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), C₁₋₆ alkoxy (e.g., C₁₋₄ alkoxy, such as methoxy, ethoxy, propoxy, isopropoxy, n-butoxy, sec-butoxy, or tert-butoxy), halogen (e.g., fluorine, chlorine, bromine, or iodine), amino, hydroxy, mercapto, cyano, carboxyl, C₂₋₆ alkynyl (e.g., C₂₋₄ alkynyl, such as ethynyl, propynyl or butynyl), or C₂₋₆ alkenyl (e.g., C₂₋₄ alkenyl, such as vinyl, propenyl, or butenyl). a4 represents an integer of 0-10, e.g., an integer of 0, 1, 2, 3, 4, 5, 6, 7, 8, 9, or 10. The number of substituents is not theoretically limited in any way, or is automatically limited by the size of the building units.

In some embodiments, each ring A⁵ of the compound of Formula (I) is the same or different and independently represents heterocyclylene (e.g., 4-to 20-membered heterocyclylene, such as 4-to 18-membered, 4-to 15-membered, 4-to 12-membered, 4-to 11-membered, 4-to 10-membered, 4-to 9-membered, 4-to 8-membered, 4-to 7-membered, 4-to 6-membered, 5-to 15-membered, or 5-to 9-membered heterocyclylene), cycloalkylene (e.g., C₃₋₂₀cycloalkylene), arylene (e.g., C₅₋₂₀ arylene, such as C₆₋₂₀ arylene, C₅₋₁₅ arylene, or C₆₋₁₅ arylene), or heteroarylene (e.g., 5-to 20-membered heteroarylene, such as 5-to 15-membered, 5-to 10-membered, 5-to 9-membered, 5-to 8-membered, 5-to 7-membered, or 5-to 6-membered heteroarylene). In some embodiments, the 4-to 20-membered heterocyclylene is a 4-to 20-membered (e.g., 4-to 15-membered, 4-to 12-membered, 4-to 11-membered, 4-to 10-membered, 4-to 9-membered, 4-to 8-membered, 4-to 7-membered, 4-to 6-membered, 5-to 15-membered, or 5-to 9-membered) saturated or partially unsaturated (i.e., containing one or more double bonds, but not having a fully conjugated x-electron system) divalent monocyclic, bicyclic, tricyclic or polycyclic cyclic hydrocarbon group containing one or more (e.g., from 1 to 5, from 1 to 4, or from 1 to 3, or from 1 to 2, or 1) heteroatoms selected from the group consisting of oxygen, nitrogen, and sulfur. Examples of the 4-to 20-membered heterocyclylene include, but are not limited to, monocyclic heterocyclylene (e.g., 4-to 20-membered monocyclic heterocyclylene), bridged heterocyclylene (e.g., 5-to 20-membered bridged heterocyclylene, or 7-to 15-membered bridged heterocyclylene), fused heterocyclylene, and spiro-heterocyclylene (e.g., 5-to 20-membered spiro-heterocyclylene), e.g., azetidinylene, oxetanylene, pyrrolidinylene, imidazolidinylene, pyrazolidylene, tetrahydrofuranylene, tetrahydropyranylene, tetrahydrothienylene, tetrahydrothiopyranylene, oxazolidinylene, thiazolidinylene, piperidinylene, piperazinylene, morpholinylene, thiomorpholinylene, azacycloheptanylene, azacyclooctylene, dioxacyclohexylene, diazacycloheptanylene, diazacyclooctylene, 6-azabicyclo[3.1.1]heptanylene, 2,5-diazabicyclo[2.2.1]heptanylene, 3,6-diazabicyclo[3.1.1]heptanylene, 3-azabicyclo[3.2.1]octanylene, 3,8-diazabicyclo[3.2.1]octanylene, 3,8-diazabicyclo[3.2.1]octanylene, 2,5-diazabicyclo[2.2.2]octanylene, and azaspirocycloalkylene (e.g., 5-to 20-membered azaspirocycloalkylene, such as 3-azaspiro[5.5]undecanylene). In some embodiments, examples of the C₃₋₂₀ cycloalkylene include, but are not limited to, e.g., cyclopropylene, cyclobutylene, cyclopentylene, cyclopentenylene, cyclohexylene, cyclohexenylene, cycloheptylene, cyclooctylene, decalinylene, octahydropentalenylene, octahydro-1H-indenylene, 2,3-dihydro-1H-indenylene, spiro-cycloalkylene (e.g., C₅₋₁₅ spiro-cycloalkylene), adamantanylene, noradamantanylene, and norbornylene. In some embodiments, examples of the 5-to 20-membered arylene include, but are not limited to, e.g., phenylene or naphthylene. In some embodiments, the 5-to 20-membered heteroarylene is a 5-to 20-membered (e.g., 5-to 15-membered, 5-to 12-membered, 5-to 9-membered, 5-to 8-membered, 5-to 7-membered, or 5-to 6-membered) heteroarylene containing one nitrogen atom and optionally containing a heteroatom(s) selected from nitrogen, oxygen, and sulfur. In some embodiments, examples of the 5-to 20-membered heteroarylene include, but are not limited to, e.g., oxazolylene, isoxazolylene, oxadiazolylene, thienylene, thiazolylene, isothiazolylene, thiadiazolylene, pyrrolylene, imidazolylene, pyrazolylene, triazolylene, pyridylene, pyrimidinylene, pyridazinylene, pyrazinylene, indolylene, isoindolylene, benzofuranylene, isobenzofuranylene, benzothienylene, indazolylene, benzimidazolylene, benzoxazolylene, benzisoxazolylene, benzothiazolylene, benzisothiazolylene, benzotriazolylene, benzo[2,1,3]oxadiazolylene, benzo[2,1,3]thiadiazolylene, benzo[1,2,3]thiadiazolylene, quinolinylene, isoquinolinylene, naphthyridinylene, cinnolinylene, quinazolinylene, quinoxalinylene, phthalazinylene, pyrazolo[1,5-a]pyridylene, pyrazolo[1,5-a]pyrimidinylene, imidazo[1,2-alpyridylene, 1H-pyrrolo[3,2-b]pyridylene, 1H-pyrrolo[2,3-b]pyridylene, pyrrolo[2,1-b]thiazolylene, and imidazo[2,1-b]thiazolylene. Each ring A⁵ is independently optionally substituted with a5 R^{y5} substituents, and each R^{y5} independently represents deuterium, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), C₁₋₆ alkoxy (e.g., C₁₋₄ alkoxy, such as methoxy, ethoxy, propoxy, isopropoxy, n-butoxy, sec-butoxy, or tert-butoxy), halogen (e.g., fluorine, chlorine, bromine, or iodine), amino, hydroxy, mercapto, cyano, carboxyl, C₂₋₆ alkynyl, or C₂₋₆ alkenyl. a5 represents an integer of 0-10, e.g., an integer of 0, 1, 2, 3, 4, 5, 6, 7, 8, 9, or 10. The number of substituents is not theoretically limited in any way, or is automatically limited by the size of the building units.

In some embodiments, y5 of the compound of Formula (I) represents an integer of 0, 1, 2, or 3.

In some embodiments, each ring A⁵ of the compound of Formula (I) is the same or different and independently represents the following optionally substituted groups:

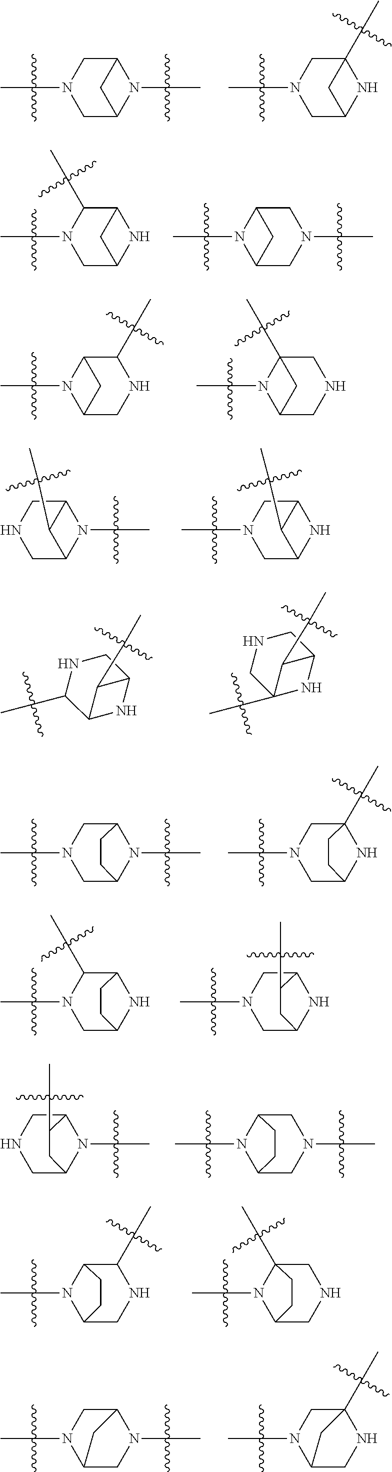

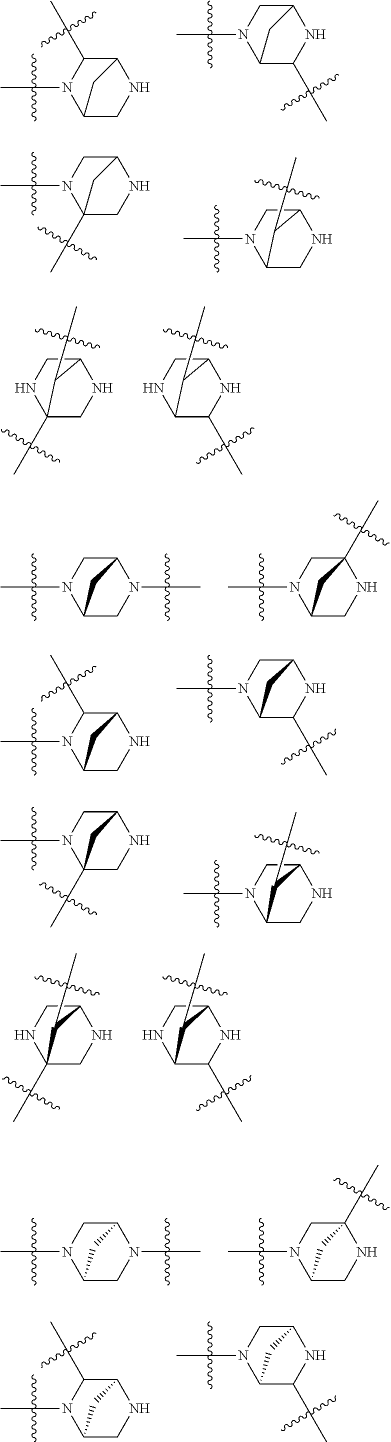

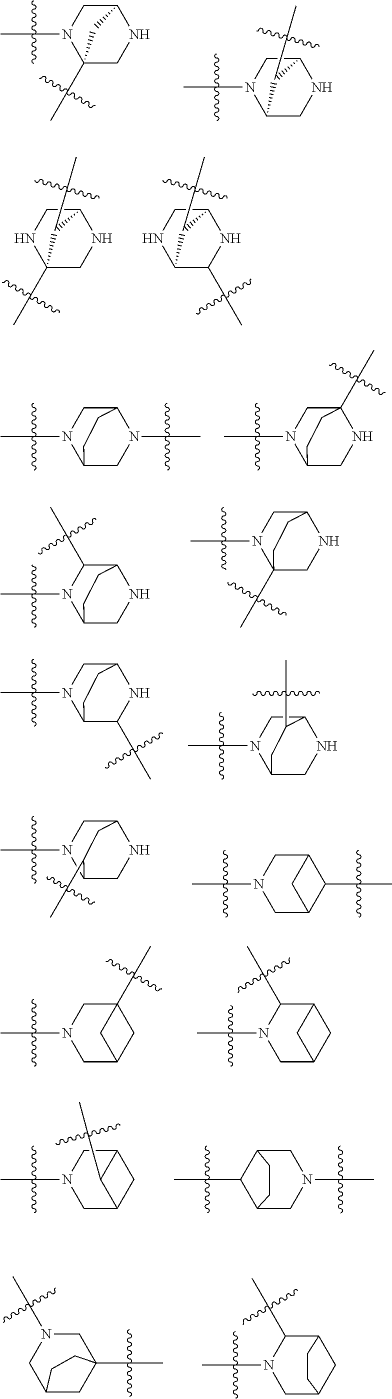

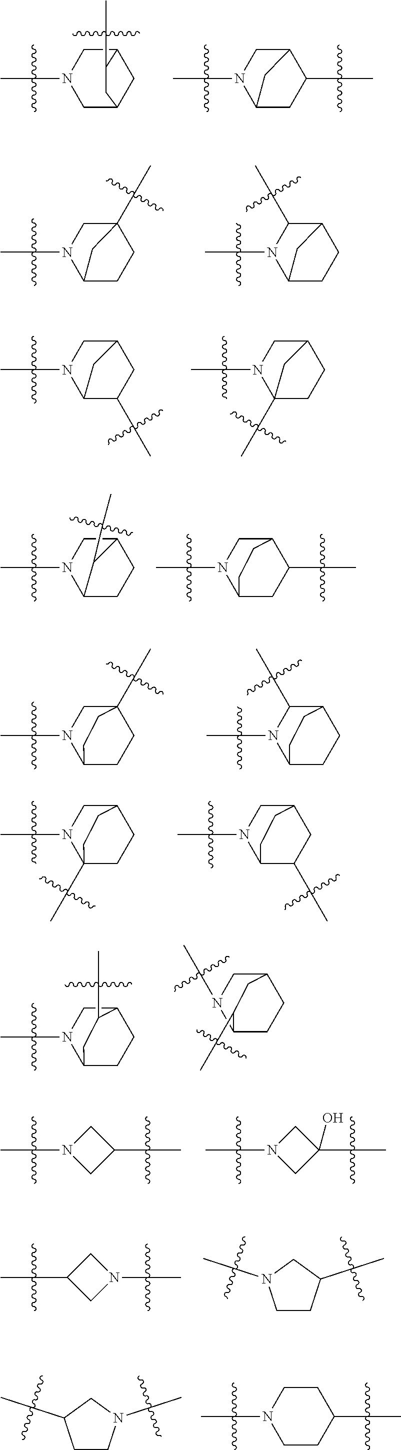

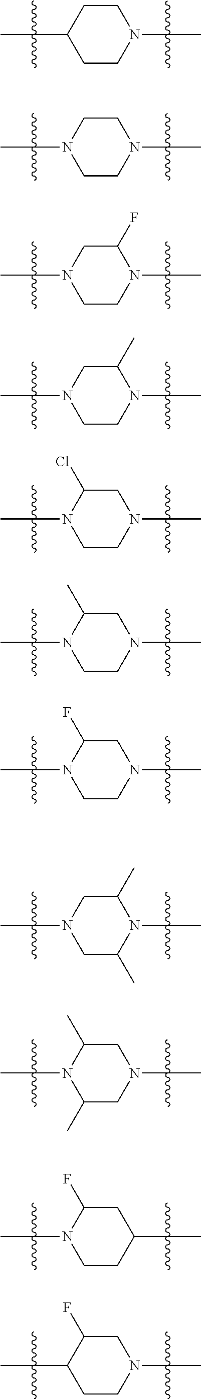

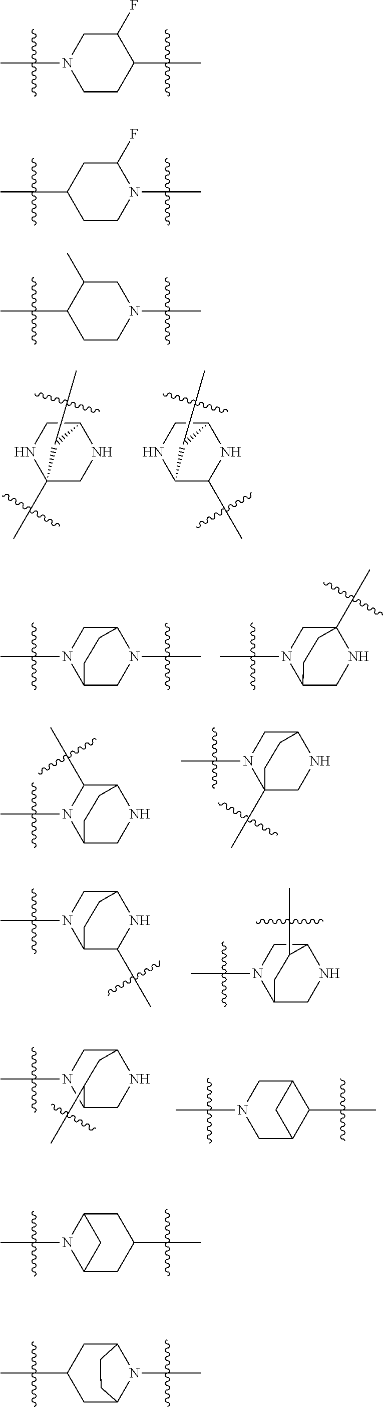

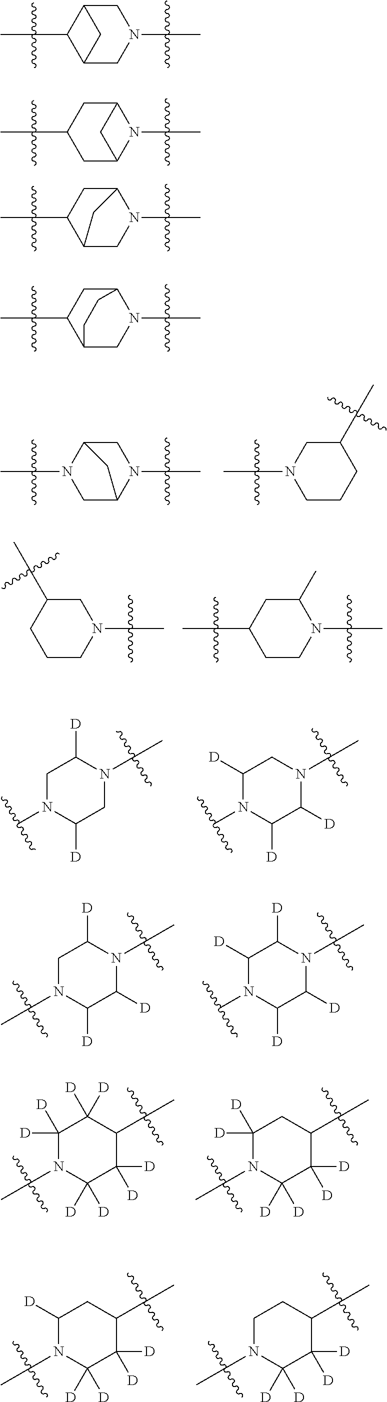

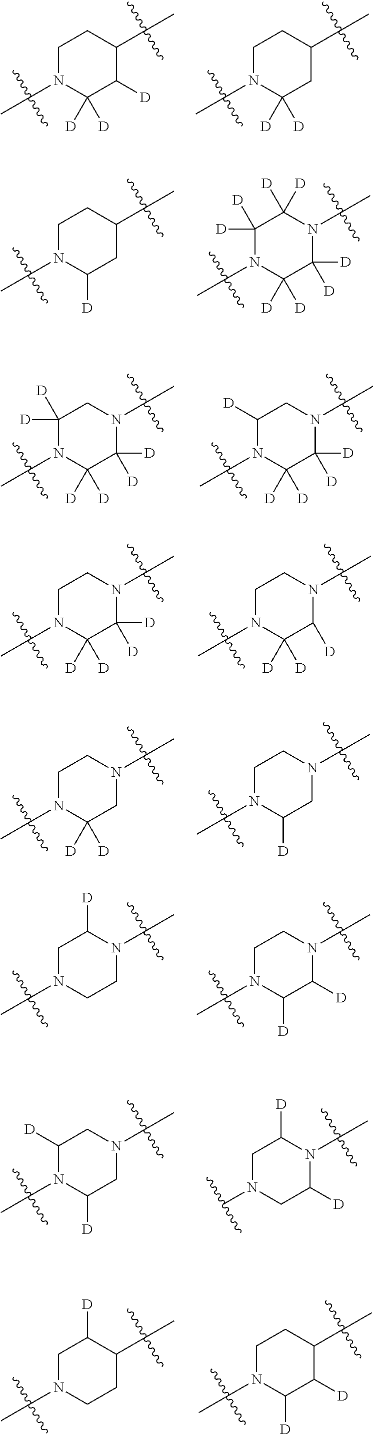

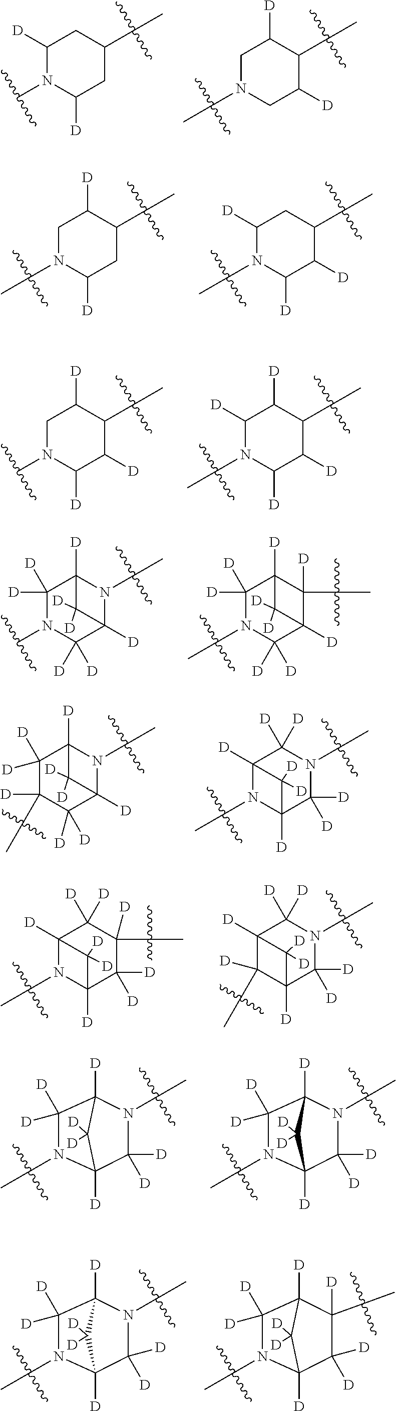

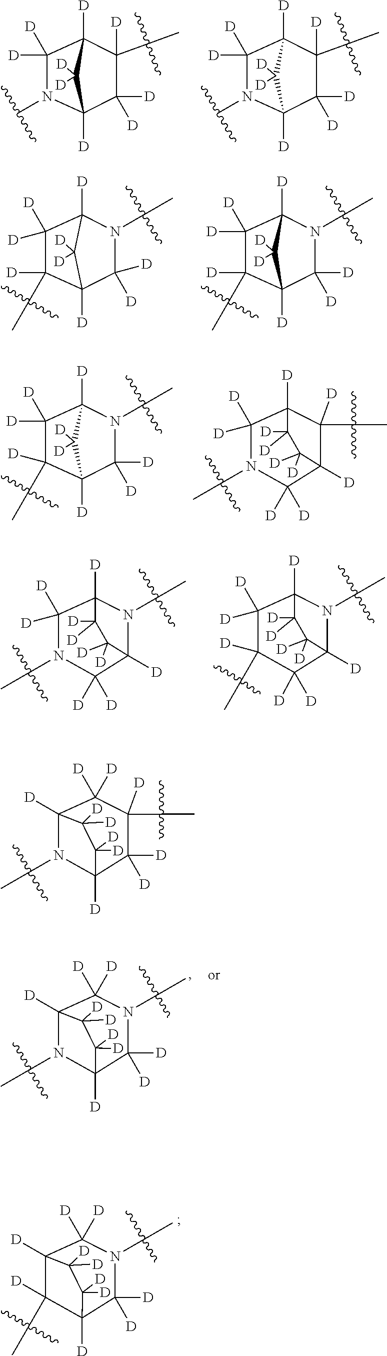

each ring A⁵ is independently optionally substituted with a5 R^{y5} groups, wherein each R^{y5} represents independently deuterium, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), C₁₋₆ alkoxy (e.g., C₁₋₄ alkoxy, such as methoxy, ethoxy, propoxy, isopropoxy, n-butoxy, sec-butoxy, or tert-butoxy), halogen (e.g., fluorine, chlorine, bromine, or iodine), amino, hydroxy, mercapto, cyano, carboxyl, C₂₋₆ alkynyl (e.g., C₂₋₄ alkynyl, such as ethynyl, propynyl or butynyl), or C₂₋₆ alkenyl (e.g., C₂₋₄ alkenyl, such as vinyl, propenyl, or butenyl). a5 represents an integer of 0-10, e.g., an integer of 0, 1, 2, 3, 4, 5, 6, 7, 8, 9, or 10. The number of substituents is not theoretically limited in any way, or is automatically limited by the size of the building units.

In some embodiments, examples of the moiety

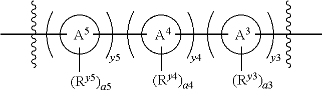

in each of the general formulae of the LIN of the compound of Formula (I) include, but are not limited to, the following groups:

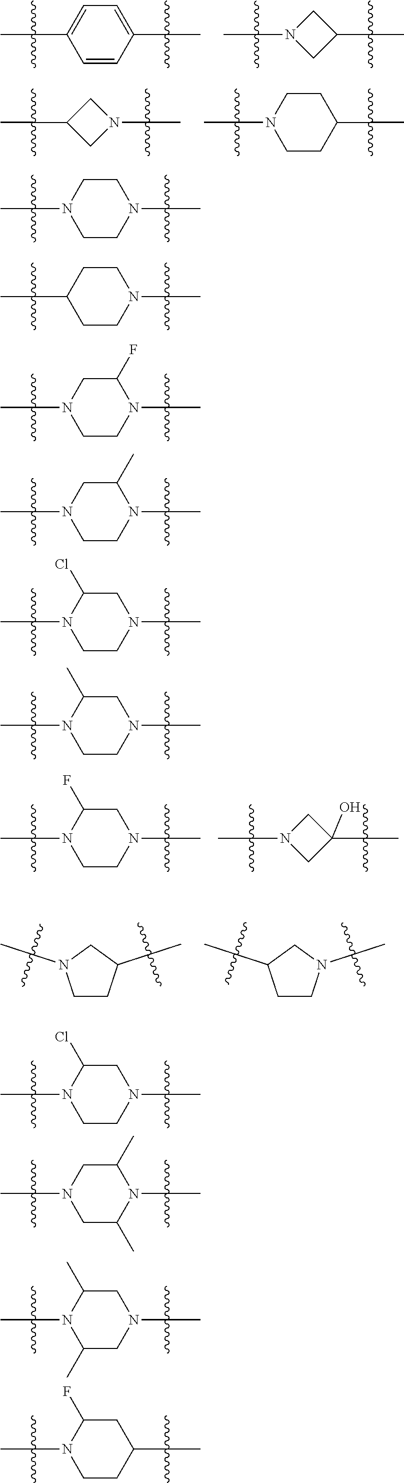

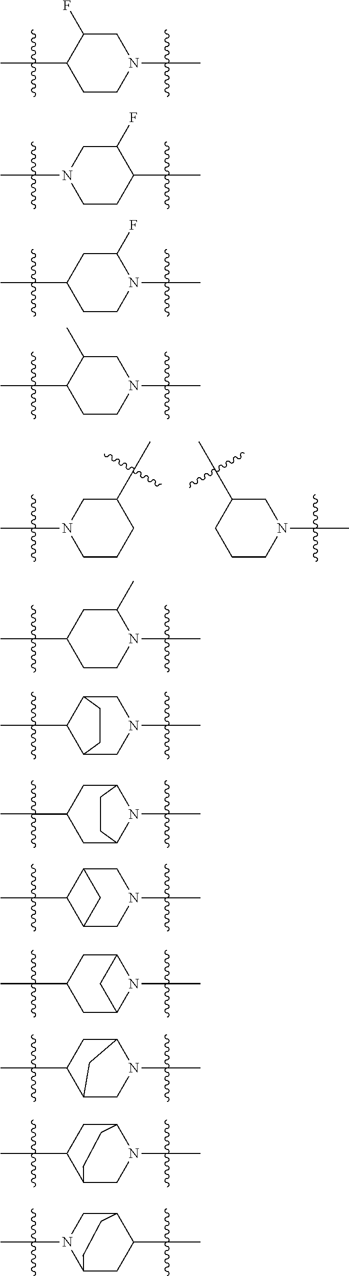

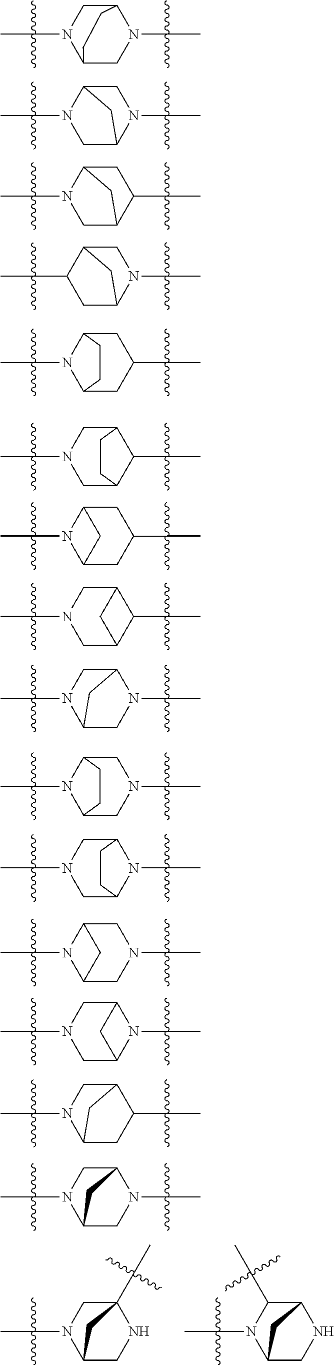

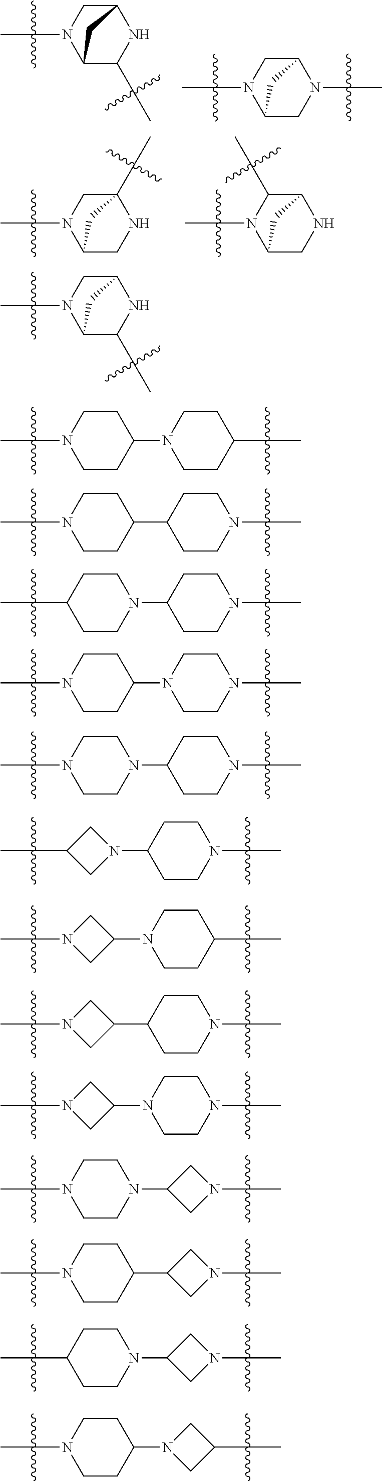

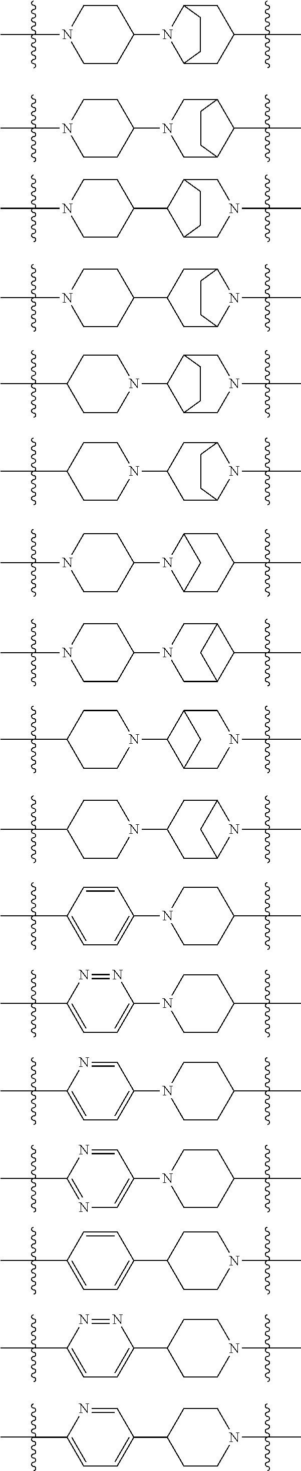

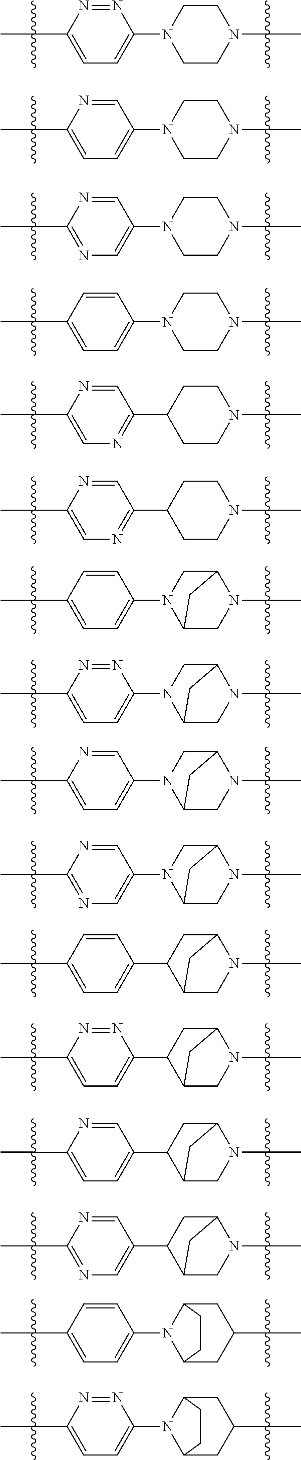

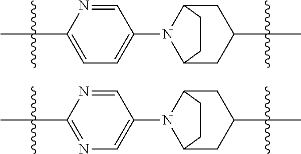

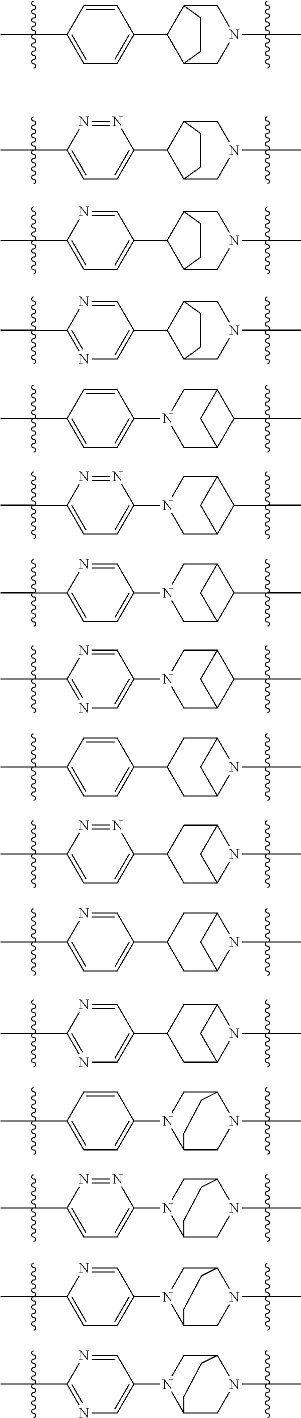

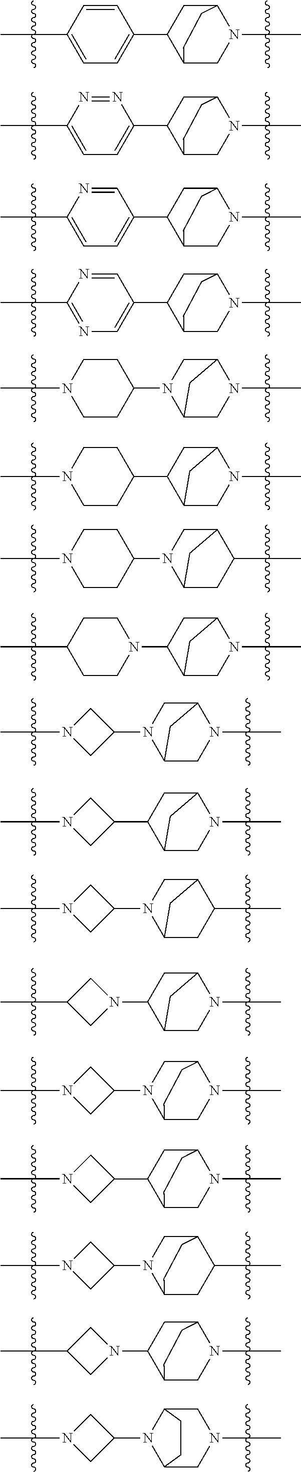

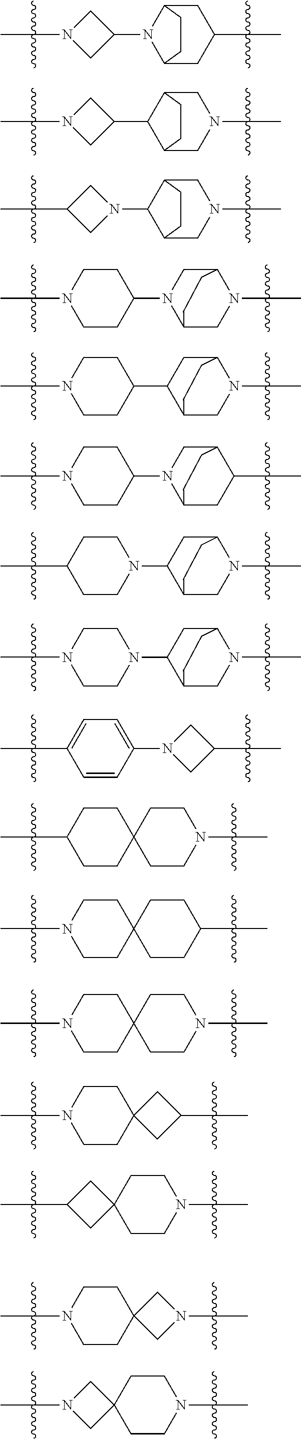

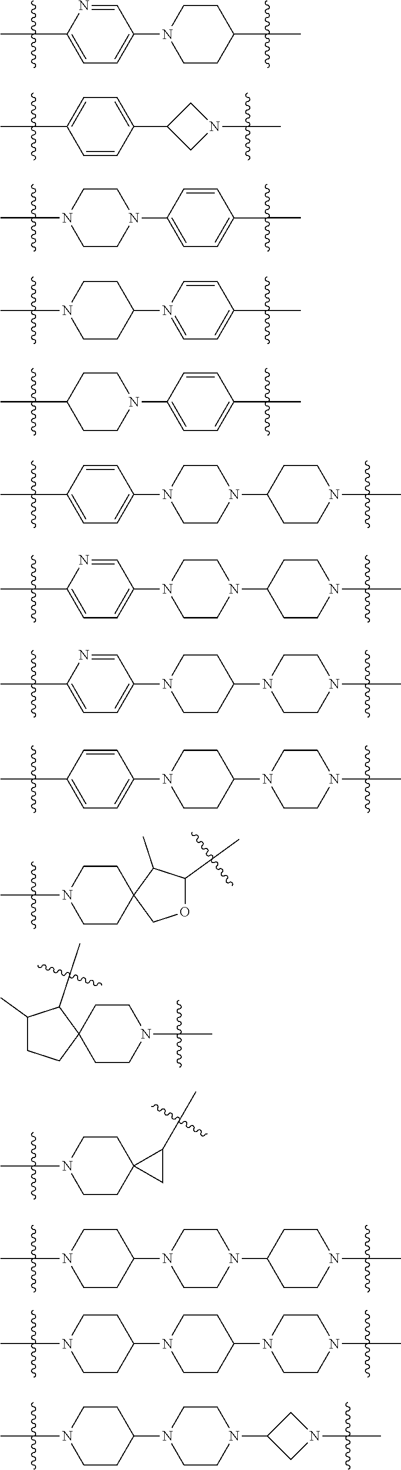

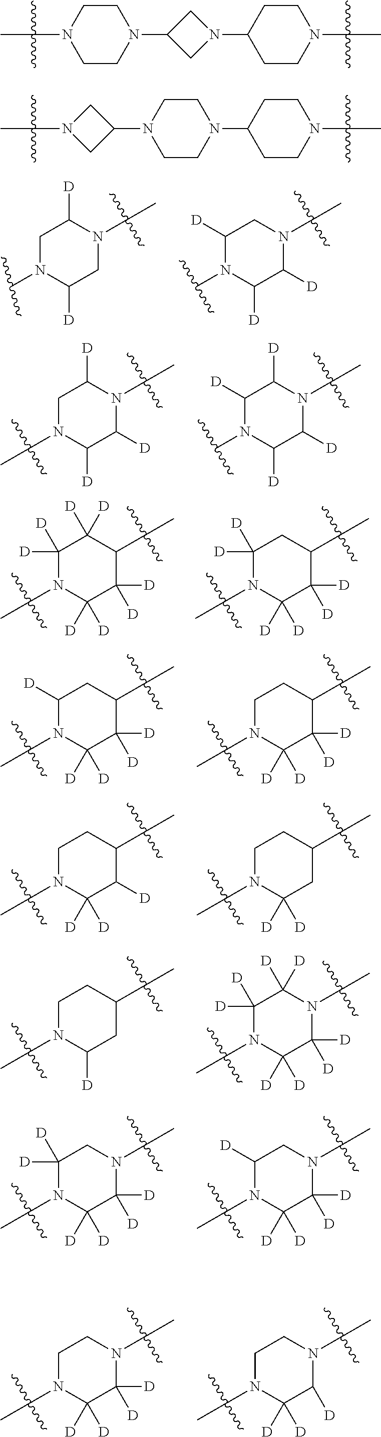

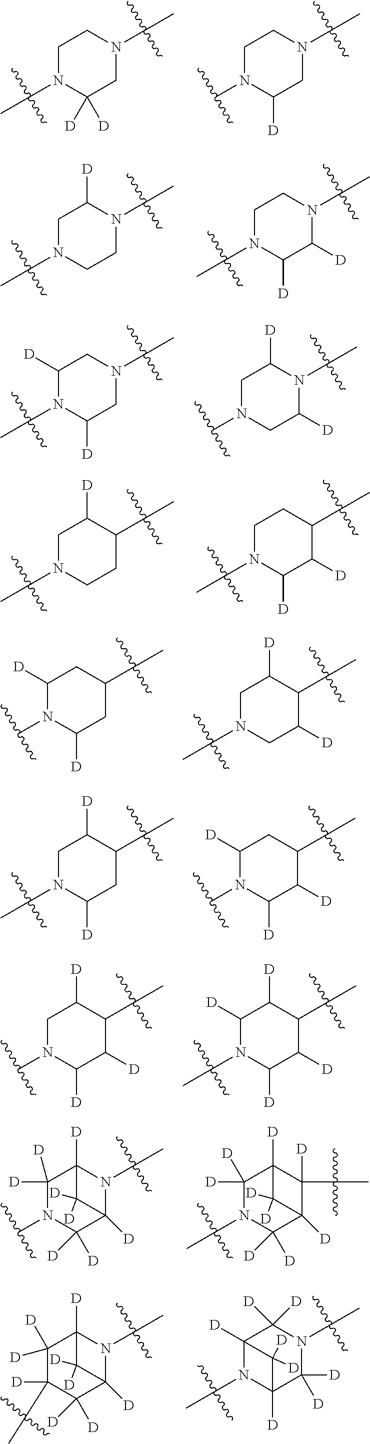

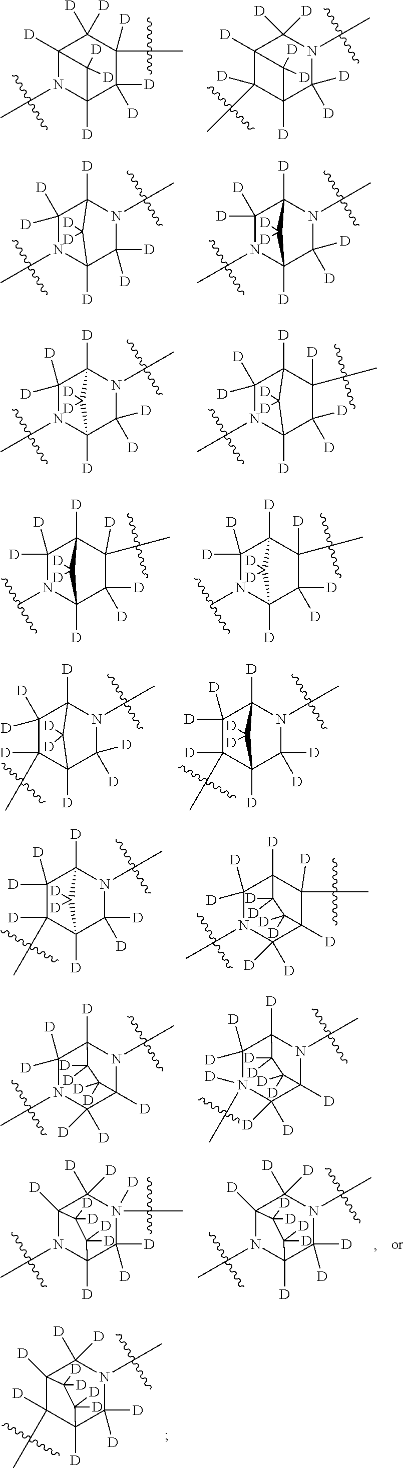

wherein the groups are optionally substituted with a substituent selected from the group consisting of deuterium, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), C₁₋₆ alkoxy (e.g., C₁₋₄ alkoxy, such as methoxy, ethoxy, propoxy, isopropoxy, n-butoxy, sec-butoxy, or tert-butoxy), halogen, amino, hydroxy, mercapto, cyano, carboxyl, C₂₋₆ alkynyl, or C₂₋₆ alkenyl. In the present disclosure, unless otherwise specified, any one end of the general formulas provided above may be connected to R_{w1} of the compound of Formula (I), while the other end is connected to R_{w2}.

In some embodiments, R_{w1} of the compound of Formula (I) represents a bond.

In some embodiments, R_{w1} of the compound of Formula (I) represents optionally substituted linear or branched C₁₋₂₀ alkylene, wherein any one or more (e.g., 1-15, 1-10, 1-9, 1-8, 1-7, 1-6, 1-5, 1-4, 1-3, or 1-2, or 1) methylene groups in the main carbon chain skeleton of the linear or branched C₁₋₂₀ alkylene are optionally substituted with one or more (e.g., 1-15, 1-10, 1-9, 1-8, 1-7, 1-6, 1-5, 1-4, 1-3, or 1-2, or 1) groups selected from: Rₐ, R_{b}, and any combination thereof; wherein each R^{a} is independently selected from the group consisting of: O, C(O), OC(O), S, S(O), S(O)₂, S(O)₂N(Rₐ₇), N(Rₐ₇)S(O)₂, C(O)N(Rₐ₇), N(Rₐ₇)C(O), N(Rₐ₇), and N(Rₐ₇)C(O)N(Rₐ₇), wherein each Raz independently represents H or C₁₋₃ alkyl, and when any two or more methylene in the main carbon chain skeleton of the linear or branched C₁₋₂₀ alkylene are replaced by two or more Rₐ groups, the Rₐ groups are not directly connected to each other; and wherein each R_{b} is independently selected from the group consisting of: optionally substituted cycloalkylene (e.g., optionally substituted C₃₋₂₀ cycloalkylene), optionally substituted arylene (e.g., optionally substituted C₅₋₂₀ arylene), optionally substituted heterocyclylene (e.g., optionally substituted 4-to 20-membered heterocyclylene), optionally substituted heteroarylene (e.g., optionally substituted 5-to 20-membered heteroarylene), alkynylene (e.g., C₂₋₆ alkynylene), alkenylene (e.g., C₂₋₆ alkenylene), and any combination thereof, wherein the cycloalkylene, arylene, heterocyclylene, and heteroarylene are each independently optionally substituted with a substituent selected from the group consisting of deuterium, hydroxy, amino, mercapto, nitro, halogen, cyano, oxo, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), C₁₋₆ alkoxy (e.g., C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), C₃₋₆ cycloalkyl, halogenated C₃₋₆ cycloalkyl, deuterated C₃₋₆ cycloalkyl, 4-to 10-membered heterocyclyl, and any combination thereof; and any one or more hydrogens of said linear or branched C₁₋₂₀ alkylene are optionally replaced by a substituent selected from: deuterium, hydroxy, amino, mercapto, nitro, halogen, cyano, deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), C₁₋₆ alkoxy (e.g., C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), C₃₋₆ cycloalkyl, halogenated C₃₋₆ cycloalkyl, deuterated C₃₋₆ cycloalkyl, 4-to 10-membered heterocyclyl, and any combination thereof.

In some embodiments, R_{w1} of the compound of Formula (I) represents a structure of the following Formulae:
—C₁₋₁₅ alkylene-;
—(C(Rₐ₈)(Rₐ₉))ₙ₁—(Rₐ(C(Rₐ₁₀)(Rₐ₁₁))ₙ₂)ₘ₃—;
—(C(Rₐ₈)(Rₐ₉))ₙ₁—(Rₐ(C(Rₐ₁₀)(Rₐ₁₁))ₙ₂)ₘ₃—(Rₐ(C(R^{a12})(R^{a13}))ₙ₃)ₘ₄—;
—(C(Rₐ₈)(Rₐ₉))ₙ₁—(Rₐ(C(Rₐ₁₀)(Rₐ₁₁))ₙ₂)ₘ₃—(Rₐ(C(R^{a12})(R^{a13}))ₙ₃)ₘ₄—(Rₐ(C(Rₐ₁₄)(Rₐ₁₅))ₙ₄)ₘ₅—;
—(C(Rₐ₈)(Rₐ₉))ₙ₁—((C(Rₐ₁₀)(Rₐ₁₁))ₙ₂Rₐ)ₘ₃—;
—(C(Rₐ₈)(Rₐ₉))ₙ₁—((C(Rₐ₁₀)(Rₐ₁₁))ₙ₂Rₐ)ₘ₃—((C(R^{a12})(Rₐ₁₃))ₙ₃Rₐ)ₘ₄—;
—(C(Rₐ₈)(Rₐ₉))ₙ₁—((C(Rₐ₁₀)(Rₐ₁₁))ₙ₂Rₐ)ₘ₃—((C(R^{a12})(R^{a13}))ₙ₃Rₐ)ₘ₄—((C(Rₐ₁₄)(Rₐ₁₅))ₙ₄Rₐ)ₘ₅—;
—(C(Rₐ₈)(Rₐ₉))ₙ₁—(R_{b}(C(Rₐ₁₀)(Rₐ₁₁))ₙ₂)ₘ₃—;
—(C(Rₐ₈)(Rₐ₉))ₙ₁—(R_{b} (C(Rₐ₁₀)(Rₐ₁₁))ₙ₂)ₘ₃—(R_{b} (C(R^{a12})(Rₐ₁₃))ₙ₃)ₘ₄—;
—(C(Rₐ₈)(Rₐ₉))ₙ₁—(R_{b} (C(Rₐ₁₀)(Rₐ₁₁))ₙ₂)ₘ₃—(R_{b} (C(R^{a12})(R^{a13}))ₙ₃)ₘ₄—(R_{b} (C(Rₐ₁₄)(Rₐ₁₅))ₙ₄)ₘ₅—;
—(C(Rₐ₈)(Rₐ₉))ₙ₁—((C(Rₐ₁₀)(Rₐ₁₁))ₙ₂R_{b})ₘ₃—;
—(C(Rₐ₈)(Rₐ₉))ₙ₁—((C(Rₐ₁₀)(Rₐ₁₁))ₙ₂R_{b})ₘ₃—((C(R^{a12})(R^{a13}))ₙ₃R_{b})ₘ₄—;
—(C(Rₐ₈)(Rₐ₉))ₙ₁—((C(Rₐ₁₀)(Rₐ₁₁))ₙ₂R_{b})ₘ₃—((C(R^{a12})(R^{a13}))ₙ₃R_{b})ₘ₄—((C(Rₐ₁₄)(Rₐ₁₅))ₙ₄R_{b})ₘ₅—;
—(C(Rₐ₈)(Rₐ₉))ₙ₁—(Rₐ—R_{b}—(C(Rₐ₁₀)(Rₐ₁₁))ₙ₂)ₘ₃—;
—(C(Rₐ₈)(Rₐ₉))ₙ₁—(Rₐ(C(Rₐ₁₀)(Rₐ₁₁))ₙ₂)ₘ₃—(R_{b} (C(R^{a12})(R^{a13}))ₙ₃)ₘ₄—;
—(C(Rₐ₈)(Rₐ₉))ₙ₁—(R_{b}—Rₐ—(C(Rₐ₁₀)(Rₐ₁₁))ₙ₂)ₘ₃—;
—(C(Rₐ₈)(Rₐ₉))ₙ₁—(R_{b} (C(Rₐ₁₀)(Rₐ₁₁))ₙ₂)ₘ₃—(Rₐ(C(R^{a12})(R^{a13}))ₙ₃)ₘ₄—;
—(C(Rₐ₈)(Rₐ₉))ₙ₁—((C(Rₐ₁₀)(Rₐ₁₁))ₙ₂—Rₐ—R_{b})ₘ₃—;
—(C(Rₐ₈)(Rₐ₉))ₙ₁—((C(Rₐ₁₀)(Rₐ₁₁))ₙ₂Rₐ)ₘ₃—((C(Rₐ₁₂)(R^{a13}))ₙ₃R_{b})ₘ₄—;
—(C(Rₐ₈)(Rₐ₉))ₙ₁—((C(Rₐ₁₀)(Rₐ₁₁))ₙ₂—R_{b}—Rₐ)ₘ₃—, or
—(C(Rₐ₈)(Rₐ₉))ₙ₁—((C(Rₐ₁₀)(Rₐ₁₁))ₙ₂R_{b})ₘ₃—((C(R^{a12})(R^{a13}))ₙ₃Rₐ)ₘ₄—;
wherein each Rₐ is independently selected from the group consisting of O, C(O), OC(O), S, S(O), S(O)₂, S(O)₂N(Rₐ₇), N(Rₐ₇)S(O)₂, C(O)N(Rₐ₇), N(Rₐ₇)C(O), N(Rₐ₇), and N(Rₐ₇)C(O)N(Rₐ₇), wherein each Rₐ₇ independently represents H or C₁₋₃ alkyl; and each R_{b} is independently selected from the group consisting of optionally substituted cycloalkylene (e.g., optionally substituted C₃₋₂₀cycloalkylene), optionally substituted arylene (e.g., optionally substituted C₅₋₂₀ arylene), optionally substituted heterocyclylene (e.g., optionally substituted 4-to 20-membered heterocyclylene), optionally substituted heteroarylene (e.g., 5-to 20-membered heteroarylene), alkynylene (e.g., C₂₋₆ alkynylene), alkenylene (e.g., C₂₋₆ alkenylene), and any combination thereof, wherein the cycloalkylene, arylene, heterocyclylene, and heteroarylene are each independently optionally substituted with a substituent selected from the group consisting of deuterium, hydroxy, amino, mercapto, nitro, halogen, cyano, oxo, C₁₋₆ alkyl, halogenated C₁₋₆ alkyl, deuterated C₁₋₆ alkyl, C₁₋₆ alkoxy, halogenated C₁₋₆ alkoxy, C₃₋₆ cycloalkyl, halogenated C₃₋₆ cycloalkyl, deuterated C₃₋₆ cycloalkyl, 4-to 10-membered heterocyclyl, and any combination thereof;
Rₐ₈, Rₐ₉, Rₐ₁₀, Rₐ₁₁, Rₐ₁₂, Rₐ₁₃, Rₐ₁₄, and Rₐ₁₅ each independently represent H, deuterium, hydroxy, amino, mercapto, nitro, halogen, cyano, oxo, halogenated C₁₋₆ alkyl, deuterated C₁₋₆ alkyl, C₁₋₆ alkoxy, halogenated C₁₋₆ alkoxy, C₃₋₆ cycloalkyl, halogenated C₃₋₆ cycloalkyl, deuterated C₃₋₆ cycloalkyl, 4-to 10-membered heterocyclyl, and any combination thereof, and
n1, n2, n3, n4, m3, m4, and m5 each independently represent an integer of 1, 2, 3, 4, 5, 6, 7, 8, 9, or 10; and
any one or more hydrogens of said C₁₋₁₅ alkylene are optionally replaced by a substituent selected from: deuterium, hydroxy, amino, mercapto, nitro, halogen, cyano, halogenated C₁₋₆ alkyl, deuterated C₁₋₆ alkyl, C₁₋₆ alkoxy, halogenated C₁₋₆ alkoxy, C₃₋₆ cycloalkyl, halogenated C₃₋₆ cycloalkyl, deuterated C₃₋₆ cycloalkyl, 4-to 10-membered heterocyclyl, and any combination thereof. In the present disclosure, unless otherwise specified, any one end of the general formulas provided above may be connected to R_{w} of the compound of Formula (I), while the other end is connected to ring A³. The number of carbon atoms of each of the above general formulas, namely the sum of n1+n2*m3, the sum of n1+n2*m3+n3*m4, the sum of n1+n2*m3+n3*m4+n4*m5, each independently is an integer less than or equal to 20.

In some embodiments, R_{w1} of the compound of Formula (I) represents-C₁₋₁₅ alkylene-, wherein any one or more hydrogens of said C₁₋₁₅ alkylene are optionally replaced by a substituent selected from: deuterium, hydroxy, amino, mercapto, nitro, halogen, cyano, halogenated C₁₋₆ alkyl, deuterated C₁₋₆ alkyl, C₁₋₆ alkoxy, halogenated C₁₋₆ alkoxy, C₃₋₆ cycloalkyl, halogenated C₃₋₆ cycloalkyl, deuterated C₃₋₆ cycloalkyl, 4-to 10-membered heterocyclyl, and any combination thereof.

In some embodiments, R_{w1} of the compound of Formula (I) represents the following groups:
—CH₂—, —(CH₂)₂—, —(CH₂)₃—, —(CH₂)₄—, —(CH₂)₅—, —(CH₂)₆—, —(CH₂)₇—, —(CH₂)₈—, —(CH₂)₉—, —(CH₂)₁₀—, —(CH₂)₁₁—, —(CH₂)₁₂—, —(CH₂)₁₃—, —(CH₂)₁₄—, or —(CH₂)₁₅—;
   wherein any one or more hydrogens of said groups are optionally replaced by a substituent selected from: deuterium, hydroxy, amino, mercapto, nitro, halogen, cyano, halogenated C₁₋₆ alkyl, deuterated C₁₋₆ alkyl, C₁₋₆ alkoxy, halogenated C₁₋₆ alkoxy, C₃₋₆ cycloalkyl, halogenated C₃₋₆ cycloalkyl, deuterated C₃₋₆ cycloalkyl, 4-to 10-membered heterocyclyl, and any combination thereof.

In some embodiments, R_{w1} of the compound of Formula (I) represents a structure of the following Formulae:
—(C(Rₐ₈)(Rₐ₉))ₙ₁—(O(C(Rₐ₁₀)(Rₐ₁₁))ₙ₂)ₘ₃—;
—(C(Rₐ₈)(Rₐ₉))ₙ₁—(O(C(Rₐ₁₀)(Rₐ₁₁))ₙ₂)ₘ₃—(O(C(R^{a12})(R^{a13}))ₙ₃)ₘ₄—;
—(C(Rₐ₈)(Rₐ₉))ₙ₁—(O(C(Rₐ₁₀)(Rₐ₁₁))ₙ₂)ₘ₃—(((C(R^{a12})(R^{a13}))ₙ₃)ₘ₄—(O(C(Rₐ₁₄)(Rₐ₁₅))ₙ₄)ₘ₅—;
—(C(Rₐ₈)(Rₐ₉))ₙ₁—((C(Rₐ₁₀)(Rₐ₁₁))ₙ₂₀)ₘ₃—;
—(C(Rₐ₈)(Rₐ₉))ₙ₁—((C(Rₐ₁₀)(Rₐ₁₁))ₙ₂₀)ₘ₃—((C(R^{a12})(R^{a13}))ₙ₃₀)ₘ₄—;
—(C(Rₐ₈)(Rₐ₉))ₙ₁—((C(Rₐ₁₀)(Rₐ₁₁))ₙ₂₀)ₘ₃—((C(R^{a12})(R^{a13}))ₙ₃₀)ₘ₄—((C(Rₐ₁₄)(Rₐ₁₅))ₙ₄₀)ₘ₅—;
—(C(Rₐ₈)(Rₐ₉))ₙ₁—(N(Rₐ₇)(C(Rₐ₁₀)(Rₐ₁₁))ₙ₂)ₘ₃—;
—(C(Rₐ₈)(Rₐ₉))ₙ₁—(N(Rₐ₇)(C(Rₐ₁₀)(Rₐ₁₁))ₙ₂)ₘ₃—(N(Rₐ₇)(C(R^{a12})(R^{a13}))ₙ₃)ₘ₄—;
—(C(Rₐ₈)(Rₐ₉))ₙ₁—(N(Rₐ₇)(C(Rₐ₁₀)(Rₐ₁₁))ₙ₂)ₘ₃—(N(Rₐ₇)(C(R^{a12})(R^{a13}))ₙ₃)ₘ₄—(N(Rₐ₇)(C(Rₐ₁₄)(Rₐ₁₅))ₙ₄)ₘ₅—;
—(C(Rₐ₈)(Rₐ₉))ₙ₁—((C(Rₐ₁₀)(Rₐ₁₁))ₙ₂N(Rₐ₇))ₘ₃—;
—(C(Rₐ₈)(Rₐ₉))ₙ₁—((C(Rₐ₁₀)(Rₐ₁₁))ₙ₂N(Rₐ₇))ₘ₃—((C(R^{a12})(R^{a13}))ₙ₃N(Rₐ₇))ₘ₄—;
—(C(Rₐ₈)(Rₐ₉))ₙ₁—((C(Rₐ₁₀)(Rₐ₁₁))ₙ₂N(Rₐ₇))ₘ₃—((C(R^{a12})(R^{a13}))ₙ₃N(Rₐ₇))ₘ₄—((C(Rₐ₁₄)(Rₐ₁₅))ₙ₄N(Rₐ₇))ₘ₅—;
—(C(Rₐ₈)(Rₐ₉))ₙ₁—(C(O)N(Rₐ₇)—(C(Rₐ₁₀)(Rₐ₁₁))ₙ₂)ₘ₃—;
—(C(Rₐ₈)(Rₐ₉))ₙ₁—(C(O)N(Rₐ₇)—(C(Rₐ₁₀)(Rₐ₁₁))ₙ₂)ₘ₃—(C(O)N(Rₐ₇)—(C(R^{a12})(R^{a13}))ₙ₃)ₘ₄—;
—(C(Rₐ₈)(Rₐ₉))ₙ₁—(C(O)N(Rₐ₇)—(C(Rₐ₁₀)(Rₐ₁₁))ₙ₂)ₘ₃—(C(O)N(Rₐ₇)—(C(R^{a12})(R^{a13}))ₙ₃)ₘ₄—(C(O)N(Rₐ₇)—(C(Rₐ₁₄)(Rₐ₁₅))ₙ₄)ₘ₅—;
—(C(Rₐ₈)(Rₐ₉))ₙ₁—((C(Rₐ₁₀)(Rₐ₁₁))ₙ₂—C(O)N(Rₐ₇))ₘ₃—;
—(C(Rₐ₈)(Rₐ₉))ₙ₁—((C(Rₐ₁₀)(Rₐ₁₁))ₙ₂—C(O)N(Rₐ₇))ₘ₃—((C(R^{a12})(R^{a13}))ₙ₃—C(O)N(Rₐ₇))ₘ₄—;
—(C(Rₐ₈)(Rₐ₉))ₙ₁—((C(Rₐ₁₀)(Rₐ₁₁))ₙ₂—C(O)N(Rₐ₇))ₘ₃—((C(R^{a12})(Rₐ₁₃))ₙ₃—C(O)N(Rₐ₇))ₘ₄—((C(Rₐ₁₄)(Rₐ₁₅))ₙ₄—C(O)N(Rₐ₇))ₘ₅—;
—(C(Rₐ₈)(Rₐ₉))ₙ₁—(C(O)N(Rₐ₇)—(C(Rₐ₁₀)(Rₐ₁₁))ₙ₂)ₘ₃—(O—(C(R^{a12})(R^{a13}))ₙ₃)ₘ₄—;
—(C(Rₐ₈)(Rₐ₉))ₙ₁—C(O)N(Rₐ₇)—(C(Rₐ₁₀)(Rₐ₁₁))ₙ₂—(O(C(R^{a12})(R^{a13}))ₙ₃)ₘ₃—;
—(C(Rₐ₈)(Rₐ₉))ₙ₁—((C(Rₐ₁₀)(Rₐ₁₁))ₙ₂—C(O)N(Rₐ₇))ₘ₃—((C(R^{a12})(R^{a13}))ₙ₃₀)ₘ₄—;
—(C(Rₐ₈)(Rₐ₉))ₙ₁—(C(Rₐ₁₀)(Rₐ₁₁))ₙ₂—C(O)N(Rₐ₇)—((C(R^{a12})(R^{a13}))ₙ₃₀)ₘ₃—;
—(C(Rₐ₈)(Rₐ₉))ₙ₁—(N(Rₐ₇)C(O)—(C(Rₐ₁₀)(Rₐ₁₁))ₙ₂)ₘ₃—;
—(C(Rₐ₈)(Rₐ₉))ₙ₁—(N(Rₐ₇)C(O)—(C(Rₐ₁₀)(Rₐ₁₁))ₙ₂)ₘ₃—(O—(C(R^{a12})(R^{a13})))ₙ₃)ₘ₄—;
—(C(Rₐ₈)(Rₐ₉))ₙ₁—(N(Rₐ₇)C(O)—(C(Rₐ₁₀)(Rₐ₁₁))ₙ₂)ₘ₃—(O—(C(R^{a12})(R^{a13}))ₙ₃)ₘ₄—(O—(C(Rₐ₁₄)(Rₐ₁₅))ₙ₄)ₘ₅—;
—(C(Rₐ₈)(Rₐ₉))ₙ₁—N(Rₐ₇)C(O)—(C(Rₐ₁₀)(Rₐ₁₁))ₙ₂—(O(C(R^{a12})(R^{a13}))ₙ₃)ₘ₃—;
—(C(Rₐ₈)(Rₐ₉))ₙ₁—N(Rₐ₇)C(O)N(Rₐ₇)—(C(Rₐ₁₀)(Rₐ₁₁))ₙ₂—;
—(C(Rₐ₈)(Rₐ₉))ₙ₁—((C(Rₐ₁₀)(Rₐ₁₁))ₙ₂—N(Rₐ₇)C(O))ₘ₃—;
—(C(Rₐ₈)(Rₐ₉))ₙ₁—((C(Rₐ₁₀)(Rₐ₁₁))ₙ₂—N(Rₐ₇)C(O))ₘ₃—((C(R^{a12})(R^{a13})))ₙ₃—O)ₘ₄—;
—(C(Rₐ₈)(Rₐ₉))ₙ₁—((C(Rₐ₁₀)(Rₐ₁₁))ₙ₂—N(Rₐ₇)C(O))ₘ₃—((C(R^{a12})(R^{a13}))ₙ₃₀)ₘ₄—((C(Rₐ₁₄)(Rₐ₁₅))ₙ₄₀)ₘ₅—;
—(C(Rₐ₈)(Rₐ₉))ₙ₁—(C(Rₐ₁₀)(Rₐ₁₁))ₙ₂—N(Rₐ₇)C(O)—((C(R^{a12})(R^{a13}))ₙ₃₀)ₘ₃—;
—(C(Rₐ₈)(Rₐ₉))ₙ₁-(arylene-(C(Rₐ₁₀)(Rₐ₁₁))ₙ₂)ₘ₃—;
—(C(Rₐ₈)(Rₐ₉))ₙ₁-(arylene-(C(Rₐ₁₀)(Rₐ₁₁))ₙ₂)ₘ₃-arylene-(C(R^{a12})(R^{a13}))ₙ₃—;
—(C(Rₐ₈)(Rₐ₉))ₙ₁—((C(Rₐ₁₀)(Rₐ₁₁))ₙ₂-arylene)ₘ₃-;
—(C(Rₐ₈)(Rₐ₉))ₙ₁—((C(Rₐ₁₀)(Rₐ₁₁))ₙ₂-arylene)ₘ₃-(C(R^{a12})(R^{a13}))ₙ₃-arylene-;
—(C(Rₐ₈)(Rₐ₉))ₙ₁-(heterocyclylene-(C(Rₐ₁₀)(Rₐ₁₁))ₙ₂)ₘ₃—;
—(C(Rₐ₈)(Rₐ₉))ₙ₁-(heterocyclylene-(C(Rₐ₁₀)(Rₐ₁₁))ₙ₂)ₘ₃-(heterocyclylene-(C(R^{a12})(R^{a13}))ₙ₃)ₘ₄—;
—(C(Rₐ₈)(Rₐ₉))ₙ₁—((C(Rₐ₁₀)(Rₐ₁₁))ₙ₂-heterocyclylene)ₘ₃-;
—(C(Rₐ₈)(Rₐ₉))ₙ₁—((C(Rₐ₁₀)(Rₐ₁₁))ₙ₂-heterocyclylene)ₘ₃-((C(R^{a12})(R^{a13}))ₙ₃-heterocyclylene)ₘ₄-;
—(C(Rₐ₈)(Rₐ₉))ₙ₁-(heteroarylene-(C(Rₐ₁₀)(Rₐ₁₁))ₙ₂)ₘ₃—;
—(C(Rₐ₈)(Rₐ₉))ₙ₁-(heteroarylene-(C(Rₐ₁₀)(Rₐ₁₁))ₙ₂)ₘ₃-(heteroarylene-(C(R^{a12})(R^{a13}))ₙ₃)ₘ₄—;
—(C(Rₐ₈)(Rₐ₉))ₙ₁—((C(Rₐ₁₀)(Rₐ₁₁))ₙ₂-heteroarylene)ₘ₃-;
—(C(Rₐ₈)(Rₐ₉))ₙ₁—((C(Rₐ₁₀)(Rₐ₁₁))ₙ₂-heteroarylene)ₘ₃-((C(R^{a12})(R^{a13}))ₙ₃-heteroarylene)ₘ₄-;
—(C(Rₐ₈)(Rₐ₉))ₙ₁-(cycloalkylene-(C(Rₐ₁₀)(Rₐ₁₁))ₙ₂)ₘ₃—;
—(C(Rₐ₈)(Rₐ₉))ₙ₁-(cycloalkylene-(C(Rₐ₁₀)(Rₐ₁₁))ₙ₂)ₘ₃-(cycloalkylene-(C(R^{a12})(R^{a13}))ₙ₃)ₘ₄—;
—(C(Rₐ₈)(Rₐ₉))ₙ₁—((C(Rₐ₁₀)(Rₐ₁₁))ₙ₂-cycloalkylene)ₘ₃-, or
(C(Rₐ₈)(Rₐ₉))ₙ₁—((C(Rₐ₁₀)(Rₐ₁₁))ₙ₂-cycloalkylene)ₘ₃-((C(R^{a12})(R^{a13}))ₙ₃-cycloalkylene)ₘ₄-;
wherein each Rₐ₇ independently represents H or C₁₋₃ alkyl;
the cycloalkylene (e.g., C₃₋₂₀cycloalkylene), arylene (e.g., C₅₋₂₀ arylene), heterocyclylene (e.g., 4-to 20-membered heterocyclylene), and heteroarylene (e.g., 5-to 20-membered heteroarylene) are each independently optionally substituted with a substituent selected from the group consisting of deuterium, hydroxy, amino, mercapto, nitro, halogen, cyano, oxo, C₁₋₆ alkyl, halogenated C₁₋₆ alkyl, deuterated C₁₋₆ alkyl, C₁₋₆ alkoxy, halogenated C₁₋₆ alkoxy, C₃₋₆ cycloalkyl, halogenated C₃₋₆ cycloalkyl, deuterated C₃₋₆ cycloalkyl, 4-to 10-membered heterocyclyl, and any combination thereof;
Rₐ₈, Rₐ₉, Rₐ₁₀, Rₐ₁₁, Rₐ₁₂, Rₐ₁₃, Rₐ₁₄, and Rₐ₁₅ each independently represent H, deuterium, hydroxy, amino, mercapto, nitro, halogen, cyano, halogenated C₁₋₆ alkyl, deuterated C₁₋₆ alkyl, C₁₋₆ alkoxy, halogenated C₁₋₆ alkoxy, C₃₋₆ cycloalkyl, halogenated C₃₋₆ cycloalkyl, deuterated C₃₋₆ cycloalkyl, 4-to 10-membered heterocyclyl, and any combination thereof, and
n1, n2, n3, n4, m3, m4, and m5 each independently represent an integer of 1, 2, 3, 4, 5, 6, 7, 8, 9, or 10. The total number of carbon atoms of alkylene in each of the above general formulas, namely the sum of n1+n2*m3, the sum of n1+n2*m3+n3*m4, the sum of n1+n2*m3+n3*m4+n4*m5, each independently is an integer less than or equal to 20.

In some embodiments, examples of the cycloalkylene in the general formula structure represented by R_{w1} of the compound of Formula (I) include, but are not limited to, C₃₋₂₀ cycloalkylene, C₃₋₁₅cycloalkylene, and C₃₋₁₁cycloalkylene, e.g., cyclopropylene, cyclobutylene, cyclopentylene, cyclopentenylene, cyclohexylene, cyclohexenylene, cycloheptylene, cyclooctylene, decalinylene, octahydropentalenylene, octahydro-1H-indenylene, 2,3-dihydro-1H-indenylene, spiro-cycloalkylene (e.g., C₅₋₁₅ spiro-cycloalkylene), adamantanylene, noradamantanylene, and norbornylene. The cycloalkylene is optionally substituted with a substituent selected from the group consisting of deuterium, hydroxy, amino, mercapto, nitro, halogen, cyano, oxo, C₁₋₆ alkyl, halogenated C₁₋₆ alkyl, deuterated C₁₋₆ alkyl, C₁₋₆ alkoxy, halogenated C₁₋₆ alkoxy, C₃₋₆ cycloalkyl, halogenated C₃₋₆ cycloalkyl, deuterated C₃₋₆ cycloalkyl, 4-to 10-membered heterocyclyl, and any combination thereof.

In some embodiments, examples of the arylene in the general formula structure represented by R_{w1} of the compound of Formula (I) include, but are not limited to, C₅₋₂₀ arylene, C₆-20 arylene, C₅₋₁₅ arylene, and C₆₋₁₅ arylene, e.g., phenylene or naphthylene. The arylene is optionally substituted with a substituent selected from the group consisting of deuterium, hydroxy, amino, mercapto, nitro, halogen, cyano, C₁₋₆ alkyl, halogenated C₁₋₆ alkyl, deuterated C₁₋₆ alkyl, C₁₋₆ alkoxy, halogenated C₁₋₆ alkoxy, C₃₋₆ cycloalkyl, halogenated C₃₋₆ cycloalkyl, deuterated C₃₋₆ cycloalkyl, 4-to 10-membered heterocyclyl, and any combination thereof.

In some embodiments, examples of the heterocyclylene in the general formula structure represented by R_{w1} of the compound of Formula (I) include, but are not limited to, 4-to 20-membered, 4-to 15-membered, 4-to 12-membered, 4-to 11-membered, 4-to 10-membered, 4-to 9-membered, 4-to 8-membered, 4-to 7-membered, 4-to 6-membered, 5-to 15-membered, and 5-to 9-membered heterocyclylene, e.g., azetidinylene, oxetanylene, pyrrolidinylene, imidazolidinylene, pyrazolidylene, tetrahydrofuranylene, tetrahydropyranylene, tetrahydrothienylene, tetrahydrothiopyranylene, oxazolidinylene, thiazolidinylene, piperidinylene, piperazinylene, morpholinylene, thiomorpholinylene, azacycloheptanylene, azacyclooctylene, dioxacyclohexylene, azacycloheptanylene, azacyclooctylene, diazacycloheptanylene, diazacyclooctylene, 6-azabicyclo[3.1.1]heptanylene, 2,5-diazabicyclo[2.2.1]heptanylene, 3,6-diazabicyclo[3.1.1]heptanylene, 3-azabicyclo[3.2.1]octanylene, 3,8-diazabicyclo[3.2.1]octanylene, 3,8-diazabicyclo[3.2.1]octanylene, 2,5-diazabicyclo[2.2.2]octanylene, and 5-to 20-membered azaspirocycloalkylene. The heterocyclylene is optionally substituted with a substituent selected from the group consisting of deuterium, hydroxy, amino, mercapto, nitro, halogen, cyano, oxo, C₁₋₆ alkyl, halogenated C₁₋₆ alkyl, deuterated C₁₋₆ alkyl, C₁₋₆ alkoxy, halogenated C₁₋₆ alkoxy, C₃₋₆ cycloalkyl, halogenated C₃₋₆ cycloalkyl, deuterated C₃₋₆ cycloalkyl, 4-to 10-membered heterocyclyl, and any combination thereof.

In some embodiments, examples of the heteroarylene in the general formula structure represented by R_{w1} of the compound of Formula (I) include, but are not limited to, 5-to 20-membered heteroarylene, 5-to 15-membered, 5-to 10-membered, 5-to 9-membered, 5-to 8-membered, 5-to 7-membered, and 5-to 6-membered heteroarylene, e.g., oxazolylene, isoxazolylene, oxadiazolylene, thienylene, thiazolylene, isothiazolylene, thiadiazolylene, pyrrolylene, imidazolylene, pyrazolylene, triazolylene, pyridylene, pyrimidinylene, pyridazinylene, pyrazinylene, indolylene, isoindolylene, benzofuranylene, isobenzofuranylene, benzothienylene, indazolylene, benzimidazolylene, benzoxazolylene, benzisoxazolylene, benzothiazolylene, benzisothiazolylene, benzotriazolylene, benzo[2,1,3]oxadiazolylene, benzo[2,1,3]thiadiazolylene, benzo[1,2,3]thiadiazolylene, quinolinylene, isoquinolinylene, naphthyridinylene, cinnolinylene, quinazolinylene, quinoxalinylene, phthalazinylene, pyrazolo[1,5-a]pyridylene, pyrazolo[1,5-a]pyrimidinylene, imidazo[1,2-a]pyridylene, 1H-pyrrolo[3,2-b]pyridylene, 1H-pyrrolo[2,3-b]pyridylene, pyrrolo[2,1-b]thiazolylene, and imidazo[2,1-b]thiazolylene. The heteroarylene is optionally substituted with a substituent selected from the group consisting of deuterium, hydroxy, amino, mercapto, nitro, halogen, cyano, C₁₋₆ alkyl, halogenated C₁₋₆ alkyl, deuterated C₁₋₆ alkyl, C₁₋₆ alkoxy, halogenated C₁₋₆ alkoxy, C₃₋₆ cycloalkyl, halogenated C₃₋₆ cycloalkyl, deuterated C₃₋₆ cycloalkyl, 4-to 10-membered heterocyclyl, and any combination thereof.

In some embodiments, examples of R_{w1} of the compound of Formula (I) include, but are not limited to, the following groups:
O—CH₂—, —O—(CH₂)₂—, —O—(CH₂)₃—, —O—(CH₂)₄—, —O—(CH₂)₅—, —O—(CH₂)₆—, —O—(CH₂)₇—, —O—(CH₂)₈—, —O—(CH₂)₉—, —O—(CH₂)₁₀—, —(CH₂)₁—O—, —(CH₂)₂—O—, —(CH₂)₃—O—, —(CH₂)₄—O—, —(CH₂)₅—O—, —(CH₂)₆—O—, —(CH₂)₇—O—, —(CH₂)₈—O—, (CH₂)₉—O—, —(CH₂)₁₀—O—, —CH₂—O—CH₂—, —CH₂—O—(CH₂)₂—, —(CH₂)₁—O—(CH₂)₃—, —(CH₂)₁—O—(CH₂)₄—, —(CH₂)₁—O—(CH₂)₅—, —(CH₂)₁—O—(CH₂)₆—, —(CH₂)₁—O—(CH₂)₇—, —(CH₂)₁—O—(CH₂)₈—, —(CH₂)₁—O—(CH₂)₉—, —(CH₂)₁—O—(CH₂)₁₀—, —(CH₂)₂—O—(CH₂)₁—, —(CH₂)₂—O—(CH₂)₂—, —(CH₂)₂—O—(CH₂)₃—, —(CH₂)₂—O—(CH₂)₄—, —(CH₂)₂—O—(CH₂)₅—, —(CH₂)₂—O—(CH₂)₆—, —(CH₂)₂—O—(CH₂)₇—, —(CH₂)₂—O—(CH₂)₈—, —(CH₂)₂—O—(CH₂)₉—, —(CH₂)₂—O—(CH₂)₁₀—, —(CH₂)₃—O—(CH₂)₁—, —(CH₂)₃—O—(CH₂)₂—, —(CH₂)₃—O—(CH₂)₃—, —(CH₂)₃—O—(CH₂)₄—, —(CH₂)₃—O—(CH₂)₅—, —(CH₂)₃—O—(CH₂)₆—, —(CH₂)₃—O—(CH₂)₇—, —(CH₂)₄—O—(CH₂)₁—, —(CH₂)₄—O—(CH₂)₂—, —(CH₂)₄—O—(CH₂)₃—, —(CH₂)₄—O—(CH₂)₄—, —(CH₂)₄—O—(CH₂)₅—, —(CH₂)₄—O—(CH₂)₆—, —(CH₂)₅—O—(CH₂)₁—, —(CH₂)₅—O—(CH₂)₂—, —(CH₂)₅—O—(CH₂)₃—, —(CH₂)₅—O—(CH₂)₄—, —(CH₂)₅—O—(CH₂)₅—, —(CH₂)₆—O—(CH₂)₁—, —(CH₂)₆—O—(CH₂)₂—, —(CH₂)₆—O—(CH₂)₃—, —(CH₂)₆—O—(CH₂)₄—, —(CH₂)₇—O—(CH₂)₁—, —(CH₂)₇—O—(CH₂)₂—, —(CH₂)₇—O—(CH₂)₃—, —(CH₂)₈—O—(CH₂)₁—, —(CH₂)₈—O—(CH₂)₂—, —CH(CH₃)—O—(CH₂)₁—, —CH(CH₃)—O—(CH₂)₂—, —CH(CH₃)—O—(CH₂)₃—, —CH(CH₃)—O—(CH₂)₄—, —CH(CH₃)—O—(CH₂)₅—, —CH(CH₃)—O—(CH₂)₆—, —CH(CH₃)—O—(CH₂)₇—, —CH(CH₃)—O—(CH₂)₈—, —CH(CH₃)—O—(CH₂)₉—, —CH(CH₃)—O—(CH₂)₁₀—, —(O(CH₂)₂)₂—, —(O(CH₂)₂)₃—, —(O(CH₂)₂)₄—, —(O(CH₂)₂)₅—, —(O(CH₂)₂)₆—, —(O(CH₂)₂)₇—, —CH₂—(O(CH₂)₂)₂—, —CH₂—(O(CH₂)₂)₃—, —CH₂—(O(CH₂)₂)₄—, —CH₂—(O(CH₂)₂)₅—, —CH₂—(O(CH₂)₂)₆—, —CH₂—(O(CH₂)₂)₇—, —CH₂—(O(CH₂)₂)₁—OCH₂—, —CH₂—(O(CH₂)₂)₂—OCH₂—, —CH₂—(O(CH₂)₂)₃—OCH₂—, —CH₂—(O(CH₂)₂)₄—OCH₂—, —CH₂—(O(CH₂)₂)₅—OCH₂—, —(CH₂)₂—(O(CH₂)₂)₂—, —(CH₂)₂—(O(CH₂)₂)₃—, —(CH₂)₂—(O(CH₂)₂)₄—, —(CH₂)₂—(O(CH₂)₂)₅—, —(CH₂)₂—(O(CH₂)₂)₆—, —(CH₂)₃—(O(CH₂)₂)₂—, —(CH₂)₃—(O(CH₂)₂)₃—, —(CH₂)₃—(O(CH₂)₂)₄—, —(CH₂)₃—(O(CH₂)₂)₅—, —(CH₂)₄—(O(CH₂)₂)₂—, —(CH₂)₄—(O(CH₂)₂)₃—, —(CH₂)₄—(O(CH₂)₂)₄—, —(CH₂)₄—(O(CH₂)₂)₅—, —CH₂—(O(CH₂)₃)₂—, —CH₂—(O(CH₂)₃)₃—, —CH₂—(O(CH₂)₃)₄—, —(CH₂)₂—(O(CH₂)₃)₂—, —(CH₂)₂—(O(CH₂)₃)₃—, —(CH₂)₂—(O(CH₂)₃)₄—, —(CH₂)₃—(O(CH₂)₃)₂—, —(CH₂)₃—(O(CH₂)₃)₃—, —(CH₂)₃—(O(CH₂)₃)₄—, —CH₂—O—(CH₂)₂—O—(CH₂)₃—, —CH₂—(O(CH₂)₂)₂—(O(CH₂)₃)₂—, (CH₂)₂—O—(CH₂)₂—O—(CH₂)₃—, —(CH₂)₂—(O(CH₂)₂)₂—(O(CH₂)₃)₂—, —(CH₂)₃—O—(CH₂)₂—O—(CH₂)₃—, —(CH₂)₃—(O(CH₂)₂)₂—(O(CH₂)₃)₂—, —CH₂—O—(CH₂)₃—O—(CH₂)₂—, —CH₂—(O(CH₂)₃)₂—(O(CH₂)₂)₂—, —(CH₂)₂—O—(CH₂)₃—O—(CH₂)₂—, —(CH₂)₂—(O(CH₂)₃)₂—(O(CH₂)₂)₂—, —(CH₂)₃—O—(CH₂)₃—O—(CH₂)₂—, —(CH₂)₃—(O(CH₂)₃)₂—(O(CH₂)₂)₂—, —CH₂—O—(CH₂)₂—O—CH₂—, —(CH₂)₂—O—(CH₂)₂—O—CH₂—, —(CH₂)₂—(O(CH₂)₂)₂—O—(CH₂)₃—, —(CH₂)₂—(O(CH₂)₂)₃—O—(CH₂)₃—, —(CH₂)₂—(O(CH₂)₂)₄—O—(CH₂)₃—, —(CH₂)₅—(O(CH₂)₂)₂—O—(CH₂)₅—, —(CH₂)₅—(O(CH₂)₂)₂—O—(CH₂)₆—, —(CH₂)₁—N(Rₐ₇)—(CH₂)₁—, —(CH₂)₁—N(Rₐ₇)—(CH₂)₂—, —(CH₂)₁—N(Rₐ₇)—(CH₂)₃—, —(CH₂)₁—N(Rₐ₇)—(CH₂)₄—, —(CH₂)₁—N(Rₐ₇)—(CH₂)₅—, —(CH₂)₁—N(Rₐ₇)—(CH₂)₆—, —(CH₂)₁—N(Rₐ₇)—(CH₂)₇—, —(CH₂)₁—N(Rₐ₇)—(CH₂)₈—, —(CH₂)₁—N(Rₐ₇)—(CH₂)₉—, —(CH₂)₁—N(Rₐ₇)—(CH₂)₁₀—, —N(Rₐ₇)—(CH₂)₁—, —N(Rₐ₇)—(CH₂)₂—, —N(Rₐ₇)—(CH₂)₃—, —N(Rₐ₇)—(CH₂)₄—, —N(Rₐ₇)—(CH₂)₅—, —N(Rₐ₇)—(CH₂)₆—, —N(Rₐ₇)—(CH₂)₇—, —N(Rₐ₇)—(CH₂)₈—, —N(Rₐ₇)—(CH₂)₉—, —N(Rₐ₇)—(CH₂)₁₀—, —(CH₂)₂—N(Rₐ₇)—(CH₂)₁—, —(CH₂)₂—N(Rₐ₇)—(CH₂)₂—, —(CH₂)₂—N(Rₐ₇)—(CH₂)₃—, —(CH₂)₂—N(Rₐ₇)—(CH₂)₄—, —(CH₂)₂—N(Rₐ₇)—(CH₂)₅—, —(CH₂)₂—N(Rₐ₇)—(CH₂)₆—, —(CH₂)₂—N(Rₐ₇)—(CH₂)₇—, —(CH₂)₂—N(Rₐ₇)—(CH₂)₈—, —(CH₂)₂—N(Rₐ₇)—(CH₂)₉—, —(CH₂)₂—N(Rₐ₇)—(CH₂)₁₀—, —(CH₂)₃—N(Rₐ₇)—(CH₂)₁—, —(CH₂)₃—N(Rₐ₇)—(CH₂)₂—, —(CH₂)₃—N(Rₐ₇)—(CH₂)₃—, —(CH₂)₄—N(Rₐ₇)—(CH₂)₁—, —(CH₂)₄—N(Rₐ₇)—(CH₂)₂—, —(CH₂)₄—N(Rₐ₇)—(CH₂)₃—, —(CH₂)₄—N(Rₐ₇)—(CH₂)₄—, —(CH₂)₅—N(Rₐ₇)—(CH₂)₁—, —(CH₂)₅—N(Rₐ₇)—(CH₂)₂—, —(CH₂)₅—N(Rₐ₇)—(CH₂)₃—, —(CH₂)₅—N(Rₐ₇)—(CH₂)₄—, —(CH₂)₅—N(Rₐ₇)—(CH₂)₅—, —(CH₂)₆—N(Rₐ₇)—(CH₂)₁—, —(CH₂)₆—N(Rₐ₇)—(CH₂)₂—, —(CH₂)₆—N(Rₐ₇)—(CH₂)₃—, —(CH₂)₇—N(Rₐ₇)—(CH₂)₁—, —(CH₂)₇—N(Rₐ₇)—(CH₂)₂—, —(CH₂)₇—N(Rₐ₇)—(CH₂)₃—, —(CH₂)₈—N(Rₐ₇)—(CH₂)₁—, —(CH₂)₈—N(Rₐ₇)—(CH₂)₂—, —(CH₂)₈—N(Rₐ₇)—(CH₂)₃—, —CH(CH₃)—N(Rₐ₇)—(CH₂)₁—, —CH(CH₃)—N(Rₐ₇)—(CH₂)₂—, —CH(CH₃)—N(Rₐ₇)—(CH₂)₃—, —CH(CH₃)—N(Rₐ₇)—(CH₂)₄—, —CH(CH₃)—N(Rₐ₇)—(CH₂)₅—, —CH(CH₃)—N(Rₐ₇)—(CH₂)₆—, —CH(CH₃)—N(Rₐ₇)—(CH₂)₇—, —CH(CH₃)—N(Rₐ₇)—(CH₂)₈—, —CH(CH₃)—N(Rₐ₇)—(CH₂)₉—, —CH(CH₃)—N(Rₐ₇)—(CH₂)₁₀—, —C(O)NHCH₂—, —C(O)NH(CH₂)₂—, —C(O)NH(CH₂)₃—, —C(O)NH(CH₂)₄—, —C(O)NH(CH₂)₅—, —CH₂C(O)NHCH₂—, —(CH₂)₂C(O)NH(CH₂)₂—, —(CH₂)₂C(O)NH(CH₂)₃—, —(CH₂)₂C(O)NH(CH₂)₄—, —(CH₂)₂C(O)NH(CH₂)₅—, —(CH₂)₃C(O)NH(CH₂)₃—, —(CH₂)₃C(O)NH(CH₂)₄—, —(CH₂)₄C(O)NH(CH₂)₄—, —(CH₂)₅C(O)NH(CH₂)₅—, —(CH₂)₆C(O)NH(CH₂)₇—, —(CH₂)₆C(O)NH(CH₂)₆—, —(CH₂)₇C(O)NH(CH₂)₇—, —(CH₂)₂C(O)NH(CH₂)₂—O—(CH₂)₂—, —NHC(O)CH₂—, —NHC(O)(CH₂)₂—, —NHC(O)(CH₂)₃—, —NHC(O)(CH₂)₄—, —NHC(O)(CH₂)₅—, —CH₂NHC(O)CH₂—, —(CH₂)₂NHC(O)(CH₂)₂—, —(CH₂)₂NHC(O)(CH₂)₃—, —(CH₂)₂NHC(O)(CH₂)₄—, (CH₂)₂NHC(O)(CH₂)₅—, —(CH₂)₃NHC(O)(CH₂)₃—, —(CH₂)₃NHC(O)(CH₂)₄—, —(CH₂)₄NHC(O)(CH₂)₄—, —(CH₂)₅NHC(O)(CH₂)₅—, —(CH₂)₆NHC(O)(CH₂)₇—, —(CH₂), NHC(O)(CH₂)₆—, —(CH₂)—NHC(O)(CH₂)₇—, —(CH₂)₄NHC(O)(CH₂)₈—, —(CH₂)₂NHC(O)(CH₂)₂—O—(CH₂)₂—, —(CH₂)₄NHC(O)CH₂—,-phenylene-CH₂—, -phenylene-(CH₂)₂—,-phenylene-(CH₂)₃—,-phenylene-(CH₂)₄—,-phenylene-(CH₂)₅—,-phenylene-(CH₂)₆—, -phenylene-(CH₂)₇—,-phenylene-(CH₂)₈—, —CH₂-phenylene-CH₂—, —CH₂-phenylene-(CH₂)₂—, —CH₂-phenylene-(CH₂)₃—, —CH₂-phenylene-(CH₂)₄—, —CH₂-phenylene-(CH₂)₅—, —CH₂-phenylene-(CH₂)₆—, —CH₂-phenylene-(CH₂)₇—, —CH₂-phenylene-(CH₂)₈—, —(CH₂)₂-phenylene-(CH₂)₁—, —(CH₂)₂-phenylene-(CH₂)₂—, —(CH₂)₂-phenylene-(CH₂)₃—, —(CH₂)₂-phenylene-(CH₂)₄—, —(CH₂)₂-phenylene-(CH₂)₅—, —(CH₂)₂-phenylene-(CH₂)₆—, —(CH₂)₂-phenylene-(CH₂)₇—, (CH₂)₂-phenylene-(CH₂)₈—, —(CH₂)₃-phenylene-CH₂—, —(CH₂)₃-phenylene-(CH₂)₂—, —(CH₂)₃-phenylene-(CH₂)₃—, —(CH₂)₃-phenylene-(CH₂)₄—, —(CH₂)₃-phenylene-(CH₂)₅—, —(CH₂)₃-phenylene-(CH₂)₆—, —(CH₂)₃-phenylene-(CH₂)₇—, (CH₂)₃-phenylene-(CH₂)₈—, —(CH₂)₄-phenylene-CH₂—, —(CH₂)₄-phenylene-(CH₂)₂—, —(CH₂)₄-phenylene-(CH₂)₃—, —(CH₂)₄-phenylene-(CH₂)₄—, —(CH₂)₄-phenylene-(CH₂)₅—, —(CH₂)₄-phenylene-(CH₂)₆—, —(CH₂)₄-phenylene-(CH₂)₇—, (CH₂)₄-phenylene-(CH₂)₈—, —(CH₂)₅-phenylene-(CH₂)₁—, —(CH₂)₅-phenylene-(CH₂)₂—, —(CH₂)₅-phenylene-(CH₂)₃—, —(CH₂)₅-phenylene-(CH₂)₄—, —(CH₂)₅-phenylene-(CH₂)₅—, —(CH₂)₅-phenylene-(CH₂)₆—, —(CH₂)₅-phenylene-(CH₂)₇—, —(CH₂)₅-phenylene-(CH₂)₈—, —(CH₂)₆-phenylene-(CH₂)₁—, —(CH₂)₆-phenylene-(CH₂)₂—, —(CH₂)₆-phenylene-(CH₂)₃—, —(CH₂)₆-phenylene-(CH₂)₄—, —(CH₂)₆-phenylene-(CH₂)₅—, —(CH₂)₆-phenylene-(CH₂)₆—, (CH₂)₆-phenylene-(CH₂)₇—, —(CH₂)₆-phenylene-(CH₂)₈—, —(CH₂)₇-phenylene-(CH₂)₁—, —(CH₂)₇-phenylene-(CH₂)₂—, —(CH₂)₇-phenylene-(CH₂)₃—, —(CH₂)₇-phenylene-(CH₂)₄—, —(CH₂)₇-phenylene-(CH₂)₈—, —(CH₂)₈-phenylene-CH₂—, —(CH₂)₈-phenylene-(CH₂)₂—, —(CH₂)₈-phenylene-(CH₂)₃—, —(CH₂)₈-phenylene-(CH₂)₄—, —(CH₂)₈-phenylene-(CH₂)₅—, —(CH₂)₈-phenylene-(CH₂)₆—, —(CH₂)₈-phenylene-(CH₂)₇—, —N(Rₐ₇)—CH₂-phenylene-CH₂—, —N(Rₐ₇)—CH₂-phenylene-(CH₂)₂—, —N(Rₐ₇)—CH₂-phenylene-(CH₂)₃—, —N(Rₐ₇)—CH₂-phenylene-(CH₂)₄—, —N(Rₐ₇)—CH₂-phenylene-(CH₂)₅—, —N(Rₐ₇)—CH₂-phenylene-(CH₂)₆—, —N(Rₐ₇)—CH₂-phenylene-(CH₂)₇—, —N(Rₐ₇)—CH₂-phenylene-(CH₂)₈—, —CH₂—N(Rₐ₇)—CH₂-phenylene-CH₂—, —CH₂—N(Rₐ₇)—CH₂-phenylene-(CH₂)₂—, —CH₂—N(Rₐ₇)—CH₂-phenylene-(CH₂)₃—, —CH₂—N(Rₐ₇)—CH₂-phenylene-(CH₂)₄—, —CH₂—N(Rₐ₇)—CH₂-phenylene-(CH₂)₅—, —CH₂—N(Rₐ₇)—CH₂-phenylene-(CH₂)₆—, —CH₂—N(Rₐ₇)—CH₂-phenylene-(CH₂)₇—, —CH₂—N(Rₐ₇)—CH₂-phenylene-(CH₂)₈—, —CH₂—N(Rₐ₇)—(CH₂)₂-phenylene-CH₂—, —CH₂—N(Rₐ₇)—(CH₂)₂-phenylene-(CH₂)₂—, —CH₂—N(Rₐ₇)—(CH₂)₂- phenylene-(CH₂)₃—, —CH₂—N(Rₐ₇)—(CH₂)₂-phenylene-(CH₂)₄—, —CH₂—N(Rₐ₇)—(CH₂)₂-phenylene-(CH₂)₅—, —CH₂—N(Rₐ₇)—(CH₂)₂-phenylene-(CH₂)₆—, —CH₂—N(Rₐ₇)—(CH₂)₂-phenylene-(CH₂)₇—, —CH₂—N(Rₐ₇)—(CH₂)₂-phenylene-(CH₂)₈—, —(CH₂)₂—N(Rₐ₇)—CH₂-phenylene-CH₂—, —(CH₂)₂—N(Rₐ₇)—CH₂-phenylene-(CH₂)₂—, —(CH₂)₂—N(Rₐ₇)—CH₂-phenylene-(CH₂)₃—, (CH₂)₂—N(Rₐ₇)—CH₂-phenylene-(CH₂)₄—, —(CH₂)₂—N(Rₐ₇)—CH₂-phenylene-(CH₂)₅—, —(CH₂)₂—N(Rₐ₇)—CH₂-phenylene-(CH₂)₆—, —(CH₂)₂—N(Rₐ₇)—CH₂-phenylene-(CH₂)₇—, —(CH₂)₂—N(Rₐ₇)—CH₂-phenylene-(CH₂)₈—, —(CH₂)₃—N(Rₐ₇)—CH₂-phenylene-CH₂—, —(CH₂)₃—N(Rₐ₇)—CH₂-phenylene-(CH₂)₂—, —(CH₂)₃—N(Rₐ₇)—CH₂-phenylene-(CH₂)₃—, (CH₂)₃—N(Rₐ₇)—CH₂-phenylene-(CH₂)₈—, —(CH₂)₄—N(Rₐ₇)—CH₂-phenylene-CH₂—, —(CH₂)₄—N(Rₐ₇)—CH₂- phenylene-(CH₂)₂—, —(CH₂)₄—N(Rₐ₇)—CH₂-phenylene-(CH₂)₃—, —(CH₂)₄—N(Rₐ₇)—CH₂-phenylene-(CH₂)₈—, —(CH₂)₅—N(Rₐ₇)—CH₂-phenylene-(CH₂)₃—, —(CH₂)₅—N(Rₐ₇)—CH₂-phenylene-(CH₂)₈—, —(CH₂)₆—N(Rₐ₇)—CH₂-phenylene-(CH₂)₃—, —(CH₂)₆—N(Rₐ₇)—CH₂-phenylene-(CH₂)₈—, —(CH₂)₇—N(Rₐ₇)—CH₂-phenylene-(CH₂)₃—, —(CH₂)₈—N(Rₐ₇)—CH₂-phenylene-CH₂—, (CH₂)₈—N(Rₐ₇)—CH₂-phenylene-(CH₂)₂—, —(CH₂)₈—N(Rₐ₇)—CH₂-phenylene-(CH₂)₃—, —(CH₂)₈—N(Rₐ₇)—CH₂-phenylene-(CH₂)₄—, —(CH₂)₈—N(Rₐ₇)—CH₂-phenylene-(CH₂)₅—, —(CH₂)₈—N(Rₐ₇)—CH₂-phenylene-(CH₂)₆—, - piperidinylene-CH₂—, -piperidinylene-(CH₂)₂—, -piperidinylene-(CH₂)₃—, -piperidinylene-(CH₂)₄—, piperidinylene-(CH₂)₅—, -piperidinylene-(CH₂)₆—, -piperidinylene-(CH₂)₇—, -piperidinylene-(CH₂)₈—, —CH₂-piperidinylene-CH₂—, —CH₂-piperidinylene-(CH₂)₂—, —CH₂-piperidinylene-(CH₂)₃—, —CH₂-piperidinylene-(CH₂)₄—, —CH₂-piperidinylene-(CH₂)₅—, —CH₂-piperidinylene-(CH₂)₆—, —CH₂-piperidinylene-(CH₂)₇—, —CH₂-piperidinylene-(CH₂)₈—, —(CH₂)₂-piperidinylene-(CH₂)₁—, —(CH₂)₂-piperidinylene-(CH₂)₂—, —(CH₂)₂-piperidinylene-(CH₂)₃—, —(CH₂)₂-piperidinylene-(CH₂)₄—, —(CH₂)₂-piperidinylene-(CH₂)₅—, —(CH₂)₂-piperidinylene-(CH₂)₆—, —(CH₂)₂-piperidinylene-(CH₂)₇—, —(CH₂)₂-piperidinylene-(CH₂)₈—, —(CH₂)₃-piperidinylene-CH₂—, —(CH₂)₃-piperidinylene-(CH₂)₂—, —(CH₂)₃-piperidinylene-(CH₂)₃—, —(CH₂)₃-piperidinylene-(CH₂)₄—, —(CH₂)₃-piperidinylene-(CH₂)₅—, —(CH₂)₃-piperidinylene-(CH₂)₆—, —(CH₂)₃-piperidinylene-(CH₂)₇—, —(CH₂)₃-piperidinylene-(CH₂)₈—, —(CH₂)₄-piperidinylene-CH₂—, —(CH₂)₄-piperidinylene-(CH₂)₂—, —(CH₂)₄-piperidinylene-(CH₂)₃—, —(CH₂)₄-piperidinylene-(CH₂)₄—, —(CH₂)₄-piperidinylene-(CH₂)₅—, —(CH₂)₄-piperidinylene-(CH₂)₆—, —(CH₂)₄-piperidinylene-(CH₂)₇—, —(CH₂)₄-piperidinylene-(CH₂)₈—, —(CH₂)₅-piperidinylene-(CH₂)₁—, —(CH₂)₅-piperidinylene-(CH₂)₂—, —(CH₂)₅—(CH₂)₅-piperidinylene-(CH₂)₅—, —(CH₂)₅-piperidinylene-(CH₂)₃—, —(CH₂)₅-piperidinylene-(CH₂)₄—, (CH₂)₅-piperidinylene-(CH₂)₈—, —(CH₂)₆—, —(CH₂)₅-piperidinylene-(CH₂)₇—, piperidinylene-(CH₂)₆—, (CH₂)₆-piperidinylene-(CH₂)₂—, —(CH₂)₆—, —(CH₂)₆-piperidinylene-(CH₂)₃—, piperidinylene-(CH₂)₁—, (CH₂)₆—, —(CH₂)₆-piperidinylene-(CH₂)₆—, —(CH₂)₆-piperidinylene-(CH₂)₅—, piperidinylene-(CH₂)₄—, (CH₂)₇-piperidinylene-(CH₂)₁—, —(CH₂)₇—, —(CH₂)₆-piperidinylene-(CH₂)₈—, piperidinylene-(CH₂)₇—, (CH₂)₇-piperidinylene-(CH₂)₄—, —(CH₂)₇—, —(CH₂)₇-piperidinylene-(CH₂)₃—, piperidinylene-(CH₂)₂—, piperidinylene-(CH₂)₈—, —(CH₂)₈-piperidinylene-CH₂—, —(CH₂)₈-piperidinylene-(CH₂)₂—, —(CH₂)₈-piperidinylene-(CH₂)₃—, —(CH₂)₈-piperidinylene-(CH₂)₄—, —(CH₂)₈-piperidinylene-(CH₂)₅—, —(CH₂)₈-piperidinylene-(CH₂)₆—, —(CH₂)₈-piperidinylene-(CH₂)₇—, —N(Rₐ₇)—CH₂-piperidinylene-CH₂—, —N(Rₐ₇)—CH₂-piperidinylene-(CH₂)₂—, —N(Rₐ₇)—CH₂-piperidinylene-(CH₂)₃—, —N(Rₐ₇)—CH₂-piperidinylene-(CH₂)₄—, —N(Rₐ₇)—CH₂-piperidinylene-(CH₂)₅—, —N(Rₐ₇)—CH₂-piperidinylene-(CH₂)₆—, —N(Rₐ₇)—CH₂-piperidinylene-(CH₂)₇—, —N(Rₐ₇)—CH₂-piperidinylene-(CH₂)₈—, —CH₂—N(Rₐ₇)—CH₂-piperidinylene-CH₂—, —CH₂—N(Rₐ₇)—CH₂-piperidinylene-(CH₂)₂—, —CH₂—N(Rₐ₇)—CH₂-piperidinylene- (CH₂)₃—, —CH₂—N(Rₐ₇)—CH₂-piperidinylene-(CH₂)₄—, —CH₂—N(Rₐ₇)—CH₂-piperidinylene-(CH₂)₅—, —CH₂—N(Rₐ₇)—CH₂-piperidinylene-(CH₂)₆—, —CH₂—N(Rₐ₇)—CH₂-piperidinylene-(CH₂)₇—, —CH₂—N(Rₐ₇)—CH₂- piperidinylene-(CH₂)₈—, —CH₂—N(Rₐ₇)—(CH₂)₂-piperidinylene-CH₂—, —CH₂—N(Rₐ₇)—(CH₂)₂-piperidinylene-(CH₂)₂—, —CH₂—N(Rₐ₇)—(CH₂)₂-piperidinylene-(CH₂)₃—, —CH₂—N(Rₐ₇)—(CH₂)₂-piperidinylene-(CH₂)₄—, —CH₂—N(Rₐ₇)—(CH₂)₂-piperidinylene-(CH₂)₅—, —CH₂—N(Rₐ₇)—(CH₂)₂-piperidinylene-(CH₂)₆—, —CH₂—N(Rₐ₇)—(CH₂)₂-piperidinylene-(CH₂)₇—, —CH₂—N(Rₐ₇)—(CH₂)₂-piperidinylene-(CH₂)₈—, —(CH₂)₂—N(Rₐ₇)—CH₂-piperidinylene-CH₂—, —(CH₂)₂—N(Rₐ₇)—CH₂-piperidinylene-(CH₂)₂—, —(CH₂)₂—N(Rₐ₇)—CH₂-piperidinylene-(CH₂)₃—, —(CH₂)₂—N(Rₐ₇)—CH₂- piperidinylene-(CH₂)₄—, —(CH₂)₂—N(Rₐ₇)—CH₂-piperidinylene-(CH₂)₅—, —(CH₂)₂—N(Rₐ₇)—CH₂-piperidinylene-(CH₂)₆—, —(CH₂)₂—N(Rₐ₇)—CH₂-piperidinylene-(CH₂)₇—, —(CH₂)₂—N(Rₐ₇)—CH₂-piperidinylene-(CH₂)₈—, —(CH₂)₃—N(Rₐ₇)—CH₂-piperidinylene-CH₂—, —(CH₂)₃—N(Rₐ₇)—CH₂-piperidinylene-(CH₂)₂—, —(CH₂)₃—N(Rₐ₇)—CH₂- piperidinylene-(CH₂)₃—, —(CH₂)₃—N(Rₐ₇)—CH₂-piperidinylene-(CH₂)₈—, —(CH₂)₄—N(Rₐ₇)—CH₂-piperidinylene-CH₂—, (CH₂)₄—N(Rₐ₇)—CH₂-piperidinylene-(CH₂)₂—, —(CH₂)₄—N(Rₐ₇)—CH₂-piperidinylene-(CH₂)₃—, —(CH₂)₄—N(Rₐ₇)—CH₂-piperidinylene-(CH₂)₈—, —(CH₂)₅—N(Rₐ₇)—CH₂-piperidinylene-(CH₂)₃—, —(CH₂)₅—N(Rₐ₇)—CH₂-piperidinylene-(CH₂)₈—, —(CH₂)₆—N(Rₐ₇)—CH₂-piperidinylene-(CH₂)₃—, —(CH₂)₆—N(Rₐ₇)—CH₂-piperidinylene-(CH₂)₈—, —(CH₂)₇—N(Rₐ₇)—CH₂-piperidinylene-(CH₂)₃—, —(CH₂)₈—N(Rₐ₇)—CH₂-piperidinylene-CH₂—, —(CH₂)₈—N(Rₐ₇)—CH₂- piperidinylene-(CH₂)₂—, —(CH₂)₈—N(Rₐ₇)—CH₂-piperidinylene-(CH₂)₃—, —(CH₂)₈—N(Rₐ₇)—CH₂-piperidinylene-(CH₂)₄—, —(CH₂)₈—N(Rₐ₇)—CH₂-piperidinylene-(CH₂)₅—, —(CH₂)₈—N(Rₐ₇)—CH₂-piperidinylene-(CH₂)₆—, piperazinylene-CH₂—,-piperazinylene-(CH₂)₂—,-piperazinylene-(CH₂)₃—,-piperazinylene-(CH₂)₄—, -piperazinylene-(CH₂)₅—,-piperazinylene-(CH₂)₆—,-piperazinylene-(CH₂)₇—,-piperazinylene-(CH₂)₈—, —CH₂-piperazinylene-CH₂—, —CH₂-piperazinylene-(CH₂)₂—, —CH₂-piperazinylene-(CH₂)₃—, —CH₂-piperazinylene-(CH₂)₄—, —CH₂-piperazinylene-(CH₂)₅—, —CH₂-piperazinylene-(CH₂)₆—, —CH₂-piperazinylene-(CH₂)₇—, —CH₂-piperazinylene-(CH₂)₈—, —(CH₂)₂—, —(CH₂)₂-piperazinylene-(CH₂)₁—, —(CH₂)₂-piperazinylene-(CH₂)₂—, (CH₂)₂-piperazinylene-(CH₂)₅—, —(CH₂)₂-piperazinylene-(CH₂)₃—, —(CH₂)₂-piperazinylene-(CH₂)₄—, (CH₂)₂-piperazinylene-(CH₂)₈—, —(CH₂)₃-piperazinylene-(CH₂)₆—, —(CH₂)₂-piperazinylene-(CH₂)₇—, piperazinylene-CH₂—, —(CH₂)₃-piperazinylene-(CH₂)₂—, —(CH₂)₃-piperazinylene-(CH₂)₃—, —(CH₂)₃-piperazinylene-(CH₂)₄—, —(CH₂)₃-piperazinylene-(CH₂)₅—, —(CH₂)₃-piperazinylene-(CH₂)₆—, —(CH₂)₃-piperazinylene-(CH₂)₇—, —(CH₂)₃-piperazinylene-(CH₂)₈—, —(CH₂)₄-piperazinylene-CH₂—, piperazinylene-(CH₂)₃—, —(CH₂)₄-piperazinylene-(CH₂)₄—, piperazinylene-(CH₂)₆—, —(CH₂)₄-piperazinylene-(CH₂)₇—, piperazinylene-(CH₂)₁—, —(CH₂)₅-piperazinylene-(CH₂)₂—, piperazinylene-(CH₂)₄—, —(CH₂)₅-piperazinylene-(CH₂)₅—, piperazinylene-(CH₂)₇—, —(CH₂)₅-piperazinylene-(CH₂)₈—, piperazinylene-(CH₂)₂—, —(CH₂)₆-piperazinylene-(CH₂)₃—, piperazinylene-(CH₂)₅—, —(CH₂)₆-piperazinylene-(CH₂)₆—, piperazinylene-(CH₂)₈—, —(CH₂)₇-piperazinylene-(CH₂)₁—, (CH₂)₄-piperazinylene-(CH₂)₂—, —(CH₂)₄—(CH₂)₄-piperazinylene-(CH₂)₅—, —(CH₂)₄—(CH₂)₄-piperazinylene-(CH₂)₈—, —(CH₂)₅—(CH₂)₅-piperazinylene-(CH₂)₃—, —(CH₂)₅—(CH₂)₅-piperazinylene-(CH₂)₆—, —(CH₂)₅—(CH₂)₆-piperazinylene-(CH₂)₁—, —(CH₂)₆—(CH₂)₆-piperazinylene-(CH₂)₄—, —(CH₂)₆—(CH₂)₆-piperazinylene-(CH₂)₇—, —(CH₂)₆—(CH₂)₇-piperazinylene-(CH₂)₂—, —(CH₂)₇-piperazinylene-(CH₂)₃—, —(CH₂)₇-piperazinylene-(CH₂)₄—, —(CH₂)₈-piperazinylene-CH₂—, —(CH₂)₈-piperazinylene-(CH₂)₂—, —(CH₂)₈-piperazinylene-(CH₂)₃—, —(CH₂)₈-piperazinylene-(CH₂)₄—, —(CH₂)₈-piperazinylene-(CH₂)₅—, or —(CH₂)₈-piperazinylene-(CH₂)₆—;
wherein each Rₐ₇ independently represents H or C₁₋₃ alkyl;
the phenylene, piperazinylene and piperidinylene are each independently optionally substituted with a substituent selected from the group consisting of deuterium, hydroxy, amino, mercapto, nitro, halogen, cyano, oxo, C₁₋₆ alkyl, halogenated C₁₋₆ alkyl, deuterated C₁₋₆ alkyl, C₁₋₆ alkoxy, halogenated C₁₋₆ alkoxy, C₃₋₆ cycloalkyl, halogenated C₃₋₆ cycloalkyl, deuterated C₃₋₆ cycloalkyl, 4-to 10-membered heterocyclyl, and any combination thereof. In the present disclosure, unless otherwise specified, any one end of the general formulas provided above may be connected to R_{w} of the compound of Formula (I), while the other end is connected to ring A³. For example, in the group-O—CH₂—, the O can be connected to R_{w} of the compound of Formula (I), and the CH₂ can be connected to A³ of the compound of Formula (I), and vice versa.

In some embodiments, when R_{w2} and R_{w1} of the compound of Formula (I) both represent a bond, and y3, y4, and y5 all represent an integer of 0, then LIN represents a bond (i.e., LIN is absent), and R_{w} of the compound of Formula (I) is directly connected to the R_{c} group of the PBM.

In some embodiments, examples of LIN of the compound of Formula (I) include, but are not limited to, the following groups:
C(O), C(O)NH, NHC(O), —CH₂—, —(CH₂)₂—, —(CH₂)₃—, —(CH₂)₄—, —(CH₂)₅—, —(CH₂)₆—, —(CH₂)₇—, —(CH₂)₈—, —(CH₂)₉—, —(CH₂)₁₀—, —(CH₂)₁₁—, —(CH₂)₁₂—, —(CH₂)₁₃—, —(CH₂)₁₄—, —(CH₂)₁₅—; —O—CH₂—, —O—(CH₂)₂—, —O—(CH₂)₃—, —O—(CH₂)₄—, —O—(CH₂)₅—, —O—(CH₂)₆—, —O—(CH₂)₇—, —O—(CH₂)₈—, —O—(CH₂)₉—, —O—(CH₂)₁₀—, —(CH₂)₁—O—, —(CH₂)₂—O—, —(CH₂)₃—O—, —(CH₂)₄—O—, —(CH₂)₅—O—, —(CH₂)₆—O—, —(CH₂)₇—O—, —(CH₂)₈—O—, —(CH₂)₉—O—, —(CH₂)₁₀—O—, —CH₂—O—CH₂—, —CH₂—O—(CH₂)₂—, —(CH₂)₁—O—(CH₂)₃—, —(CH₂)₁—O—(CH₂)₄—, —(CH₂)₁—O—(CH₂)₅—, —(CH₂)₁—O—(CH₂)₆—, —(CH₂)₁—O—(CH₂)₇—, —(CH₂)₁—O—(CH₂)₈—, —(CH₂)₂—O—(CH₂)₁—, —(CH₂)₂—O—(CH₂)₂—, —(CH₂)₂—O—(CH₂)₃—, —(CH₂)₂—O—(CH₂)₄—, —(CH₂)₂—O—(CH₂)₅—, —(CH₂)₂—O—(CH₂)₆—, —(CH₂)₂—O—(CH₂)₇—, —(CH₂)₂—O—(CH₂)₈—, —(CH₂)₃—O—(CH₂)₁—, —(CH₂)₃—O—(CH₂)₂—, —(CH₂)₃—O—(CH₂)₃—, —(CH₂)₃—O—(CH₂)₄—, —(CH₂)₃—O—(CH₂)₅—, —(CH₂)₃—O—(CH₂)₆—, —(CH₂)₃—O—(CH₂)₇—, —(CH₂)₄—O—(CH₂)₁—, —(CH₂)₄—O—(CH₂)₂—, —(CH₂)₄—O—(CH₂)₃—, —(CH₂)₄—O—(CH₂)₄—, —(CH₂)₄—O—(CH₂)₅—, —(CH₂)₄—O—(CH₂)₆—, —(CH₂)₅—O—(CH₂)₁—, —(CH₂)₅—O—(CH₂)₂—, —(CH₂)₅—O—(CH₂)₃—, —(CH₂)₅—O—(CH₂)₄—, —(CH₂)₅—O—(CH₂)₅—, —(CH₂)₆—O—(CH₂)₁—, —(CH₂)₆—O—(CH₂)₂—, —(CH₂)₆—O—(CH₂)₃—, —(CH₂)₆—O—(CH₂)₄—, —(CH₂)₇—O—(CH₂)₁—, —(CH₂)₇—O—(CH₂)₂—, —(CH₂)₇—O—(CH₂)₃—, —(CH₂)₈—O—(CH₂)₁—, —(CH₂)₈—O—(CH₂)₂—, —CH(CH₃)—O—(CH₂)₁—, —CH(CH₃)—O—(CH₂)₂—, —CH(CH₃)—O—(CH₂)₃—, —CH(CH₃)—O—(CH₂)₄—, —CH(CH₃)—O—(CH₂)₅—, —CH(CH₃)—O—(CH₂)₆—, —CH(CH₃)—O—(CH₂)₇—, —CH(CH₃)—O—(CH₂)₈—, —(O(CH₂)₂)₂—, —(O(CH₂)₂)₃—, —(O(CH₂)₂)₄—, —(O(CH₂)₂)₅—, —(O(CH₂)₂)₆—, —CH₂—(O(CH₂)₂)₂—, —CH₂—(O(CH₂)₂)₃—, —CH₂—(O(CH₂)₂)₄—, —CH₂—(O(CH₂)₂)₅—, —CH₂—(O(CH₂)₂)₆—, —CH₂—(O(CH₂)₂)₁—OCH₂—, —CH₂—(O(CH₂)₂)₂—OCH₂—, —CH₂—(O(CH₂)₂)₃—OCH₂—, —CH₂—(O(CH₂)₂)₄—OCH₂—, —CH₂—(O(CH₂)₂)₅—OCH₂—, —CH₂—(O(CH₂)₂)₆—OCH₂—, —(CH₂)₂—(O(CH₂)₂)₂—, —(CH₂)₂—(O(CH₂)₂)₃—, —(CH₂)₂—(O(CH₂)₂)₄—, —(CH₂)₂—(O(CH₂)₂)₅—, (CH₂)₂—(O(CH₂)₂)₆—, —(CH₂)₃—(O(CH₂)₂)₂—, —(CH₂)₃—(O(CH₂)₂)₃—, —(CH₂)₃—(O(CH₂)₂)₄—, —(CH₂)₃—(O(CH₂)₂)₅—, —(CH₂)₄—(O(CH₂)₂)₂—, —(CH₂)₄—(O(CH₂)₂)₃—, —(CH₂)₄—(O(CH₂)₂)₄—, —(CH₂)₄—(O(CH₂)₂)₅—, —CH₂—(O(CH₂)₃)₂—, —CH₂—(O(CH₂)₃)₃—, —CH₂—(O(CH₂)₃)₄—, —(CH₂)₂—(O(CH₂)₃)₂—, —(CH₂)₂—(O(CH₂)₃)₃—, —(CH₂)₂—(O(CH₂)₃)₄—, —(CH₂)₃—(O(CH₂)₃)₂—, —(CH₂)₃—(O(CH₂)₃)₃—, —CH₂—O—(CH₂)₂—O—(CH₂)₃—, —CH₂—(O(CH₂)₂)₂—(O(CH₂)₃)₂—, (CH₂)₂—O—(CH₂)₂—O—(CH₂)₃—, —(CH₂)₂—(O(CH₂)₂)₂—(O(CH₂)₃)₂—, —(CH₂)₃—O—(CH₂)₂—O—(CH₂)₃—, —(CH₂)₃—(O(CH₂)₂)₂—(O(CH₂)₃)₂—, —CH₂—O—(CH₂)₃—O—(CH₂)₂—, —CH₂—(O(CH₂)₃)₂—(O(CH₂)₂)₂—, (CH₂)₁—N(Rₐ₇)—(CH₂)₁—, —(CH₂)₁—N(Rₐ₇)—(CH₂)₂—, —(CH₂)₁—N(Rₐ₇)—(CH₂)₃—, —(CH₂)₁—N(Rₐ₇)—(CH₂)₄—, —(CH₂)₁—N(Rₐ₇)—(CH₂)₅—, —(CH₂)₁—N(Rₐ₇)—(CH₂)₆—, —N(Rₐ₇)—(CH₂)₁—, —N(Rₐ₇)—(CH₂)₂—, —N(Rₐ₇)—(CH₂)₃—, —N(Rₐ₇)—(CH₂)₄—, —N(Rₐ₇)—(CH₂)₅—, —N(Rₐ₇)—(CH₂)₆—, —(CH₂)₂—N(Rₐ₇)—(CH₂)₁—, —(CH₂)₂—N(Rₐ₇)—(CH₂)₂—, —(CH₂)₂—N(Rₐ₇)—(CH₂)₃—, —(CH₂)₂—N(Rₐ₇)—(CH₂)₄—, —(CH₂)₂—N(Rₐ₇)—(CH₂)₅—, —(CH₂)₂—N(Rₐ₇)—(CH₂)₆—, —(CH₂)₃—N(Rₐ₇)—(CH₂)₁—, —(CH₂)₃—N(Rₐ₇)—(CH₂)₂—, —(CH₂)₃—N(Rₐ₇)—(CH₂)₃—, —(CH₂)₄—N(Rₐ₇)—(CH₂)₁—, —(CH₂)₄—N(Rₐ₇)—(CH₂)₂—, —(CH₂)₄—N(Rₐ₇)—(CH₂)₃—, —(CH₂)₄—N(Rₐ₇)—(CH₂)₄—, —(CH₂)₅—N(Rₐ₇)—(CH₂)₁—, —(CH₂)₅—N(Rₐ₇)—(CH₂)₂—, —(CH₂)₅—N(Rₐ₇)—(CH₂)₃—, —(CH₂)₅—N(Rₐ₇)—(CH₂)₄—, —(CH₂)₅—N(Rₐ₇)—(CH₂)₅—, —(CH₂)₆—N(Rₐ₇)—(CH₂)₁—, —(CH₂)₆—N(Rₐ₇)—(CH₂)₂—, —(CH₂)₆—N(Rₐ₇)—(CH₂)₃—, —(CH₂)₇—N(Rₐ₇)—(CH₂)₁—, —(CH₂)₇—N(Rₐ₇)—(CH₂)₂—, —(CH₂)₇—N(Rₐ₇)—(CH₂)₃—, —(CH₂)₈—N(Rₐ₇)—(CH₂)₁—, —(CH₂)₈—N(Rₐ₇)—(CH₂)₂—, —(CH₂)₈—N(Rₐ₇)—(CH₂)₃—, —CH(CH₃)—N(Rₐ₇)—(CH₂)₁—, —CH(CH₃)—N(Rₐ₇)—(CH₂)₂—, —CH(CH₃)—N(Rₐ₇)—(CH₂)₃—, —CH(CH₃)—N(Rₐ₇)—(CH₂)₄—, —CH(CH₃)—N(Rₐ₇)—(CH₂)₅—, —CH(CH₃)—N(Rₐ₇)—(CH₂)₆—, —C(O)NHCH₂—, —C(O)NH(CH₂)₂—, —C(O)NH(CH₂)₃—, —C(O)NH(CH₂)₄—, —C(O)NH(CH₂)₅—, —CH₂C(O)NHCH₂—, —(CH₂)₂C(O)NH(CH₂)₂—, —(CH₂)₂C(O)NH(CH₂)₃—, —(CH₂)₂C(O)NH(CH₂)₄—, —(CH₂)₂C(O)NH(CH₂)₅—, —(CH₂)₃C(O)NH(CH₂)₃—, —(CH₂)₃C(O)NH(CH₂)₄—, —(CH₂)₄C(O)NH(CH₂)₄—, —(CH₂)₅C(O)NH(CH₂)₅—, —(CH₂)₂C(O)NH(CH₂)₂—O—(CH₂)₂—, —NHC(O)CH₂—, —NHC(O)(CH₂)₂—, —NHC(O)(CH₂)₃—, —NHC(O)(CH₂)₄—, —NHC(O)(CH₂)₅—, —CH₂NHC(O)CH₂—, —(CH₂)₂NHC(O)(CH₂)₂—, —(CH₂)₂NHC(O)(CH₂)₃—, —(CH₂)₂NHC(O)(CH₂)₄—, —(CH₂)₂NHC(O)(CH₂)₅—, —(CH₂)₃NHC(O)(CH₂)₃—, —(CH₂)₃NHC(O)(CH₂)₄—, —(CH₂)₄NHC(O)(CH₂)₄—, —(CH₂)₅NHC(O)(CH₂)₅—, —(CH₂)₂NHC(O)(CH₂)₂—O—(CH₂)₂—, —(CH₂)₄NHC(O)CH₂—, —CH₂-phenylene-CH₂—, —CH₂-phenylene-(CH₂)₂—, —CH₂-phenylene-(CH₂)₃—, —CH₂-phenylene-(CH₂)₄—, —CH₂-phenylene-(CH₂)₅—, —CH₂-phenylene-(CH₂)₆—, —(CH₂)₂-phenylene-(CH₂)₁—, —(CH₂)₂-phenylene-(CH₂)₂—, —(CH₂)₂-phenylene-(CH₂)₃—, —(CH₂)₂-phenylene-(CH₂)₄—, —(CH₂)₂-phenylene-(CH₂)₅—, —(CH₂)₃-phenylene-CH₂—, —(CH₂)₃-phenylene-(CH₂)₂—, —(CH₂)₃-phenylene-(CH₂)₃—, —(CH₂)₃-phenylene-(CH₂)₄—, —(CH₂)₃-phenylene-(CH₂)₅—, —(CH₂)₄-phenylene-CH₂—, —(CH₂)₄-phenylene-(CH₂)₂—, —(CH₂)₄-phenylene-(CH₂)₃—, —(CH₂)₄-phenylene-(CH₂)₄—, —(CH₂)₄-phenylene-(CH₂)₅—, —CH₂-piperidinylene-CH₂—, —CH₂-piperidinylene-(CH₂)₂—, —CH₂-piperidinylene-(CH₂)₃—, —CH₂-piperidinylene-(CH₂)₄—, —CH₂-piperidinylene-(CH₂)₅—, —CH₂-piperidinylene-(CH₂)₆—, —(CH₂)₂-piperidinylene-(CH₂)₁—, —(CH₂)₂-piperidinylene-(CH₂)₂—, —(CH₂)₂-piperidinylene-(CH₂)₃—, —(CH₂)₂-piperidinylene-(CH₂)₄—, —(CH₂)₂-piperidinylene-(CH₂)₅—, —(CH₂)₃-piperidinylene-CH₂—, —(CH₂)₃-piperidinylene-(CH₂)₂—, —(CH₂)₃-piperidinylene-(CH₂)₃—, —(CH₂)₃-piperidinylene-(CH₂)₄—, —(CH₂)₃-piperidinylene-(CH₂)₅—, —CH₂-piperazinylene-CH₂—, —CH₂-piperazinylene-(CH₂)₂—, —CH₂-piperazinylene-(CH₂)₃—, —CH₂-piperazinylene-(CH₂)₄—, —CH₂-piperazinylene-(CH₂)₅—, —(CH₂)₂-piperazinylene-(CH₂)₁—, —(CH₂)₂-piperazinylene-(CH₂)₂—, —(CH₂)₂-piperazinylene-(CH₂)₃—, —(CH₂)₂-piperazinylene-(CH₂)₄—, —(CH₂)₂-piperazinylene-(CH₂)₅—, —(CH₂)₃-piperazinylene-CH₂—, —(CH₂)₃-piperazinylene-(CH₂)₂—, —(CH₂)₃-piperazinylene-(CH₂)₃—, —(CH₂)₃-piperazinylene-(CH₂)₄—, —(CH₂)₃-piperazinylene-(CH₂)₅—, —(CH₂)₄-piperazinylene-CH₂—, —(CH₂)₄-piperazinylene-(CH₂)₂—, —(CH₂)₄-piperazinylene-(CH₂)₃—, —(CH₂)₄-piperazinylene-(CH₂)₄—, —(CH₂)₄-piperazinylene-(CH₂)₅—, —(CH₂)₈-piperazinylene-CH₂—, —(CH₂)₈-piperazinylene-(CH₂)₂—, —(CH₂)₈-piperazinylene-(CH₂)₃—, —(CH₂)₈-piperazinylene-(CH₂)₄—, or —(CH₂)₈-piperazinylene-(CH₂)₅—;
wherein the groups are optionally substituted with a substituent selected from the group consisting of deuterium, C₁₋₆ alkyl, deuterated C₁₋₆ alkyl, halogenated C₁₋₆ alkyl, C₁₋₆ alkoxy, halogen, amino, hydroxy, mercapto, cyano, carboxyl, C₂₋₆ alkynyl, or C₂₋₆ alkenyl. In the present disclosure, unless otherwise specified, any one end of the above-mentioned groups may be connected to R_{w} of the compound of Formula (I), while the other end is connected to Re of the PBM.

In some embodiments, examples of LIN of the compound of Formula (I) include, but are not limited to, the following groups:

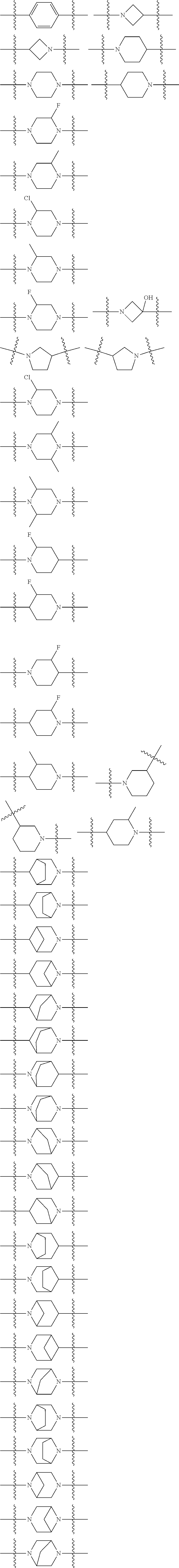

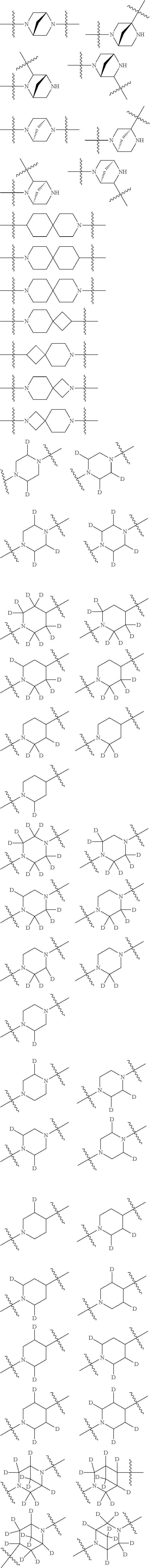

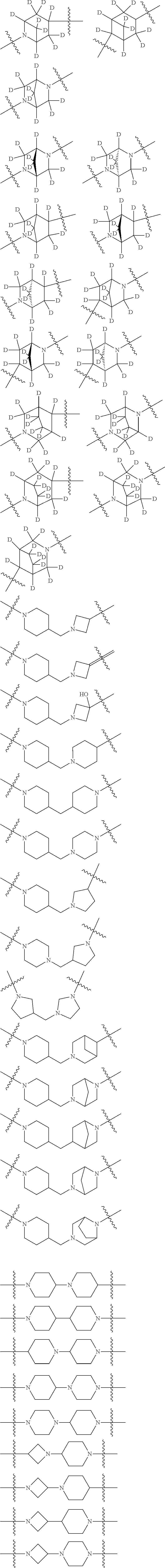

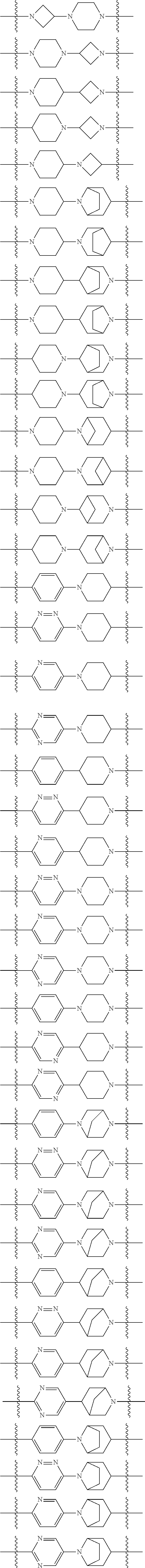

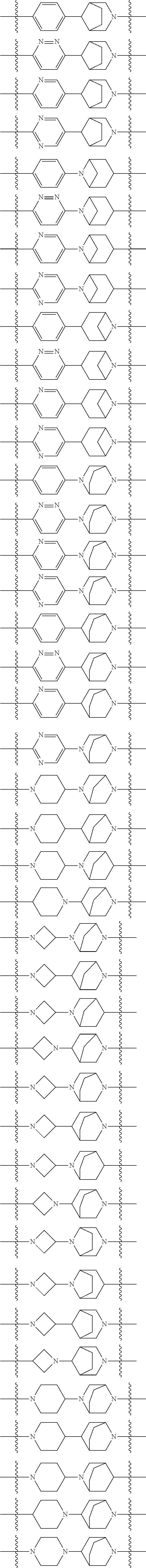

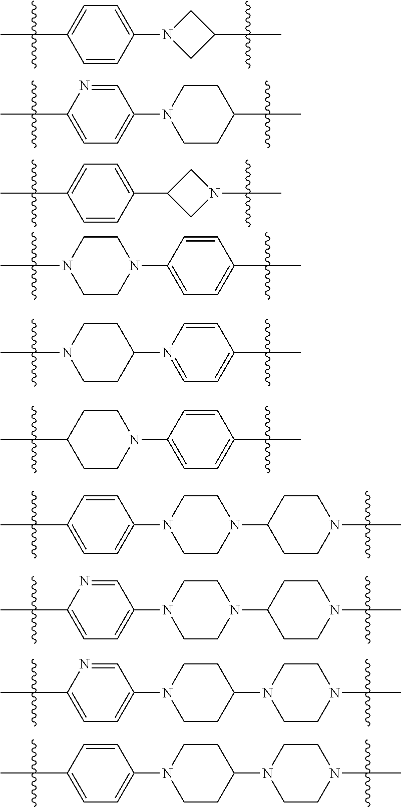

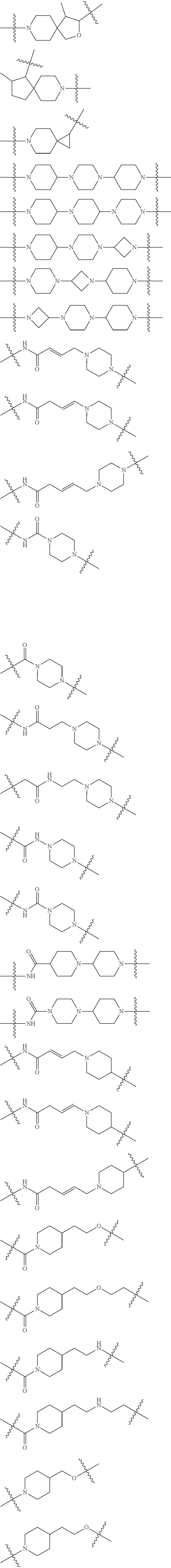

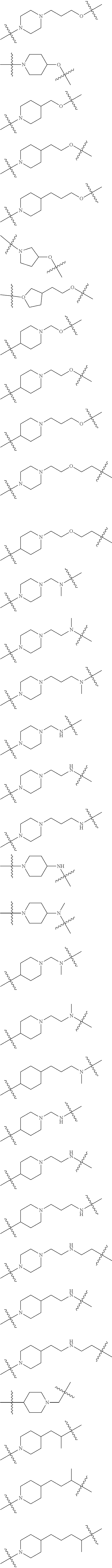

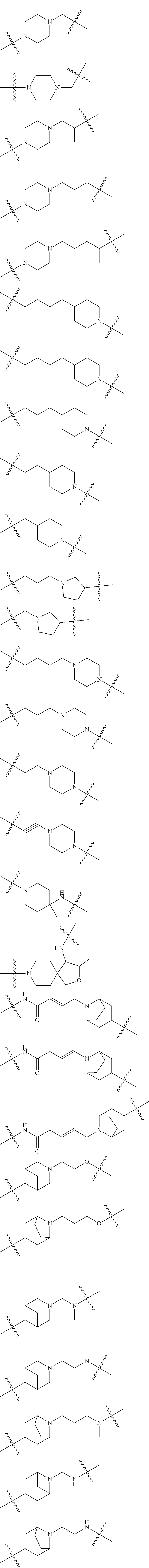

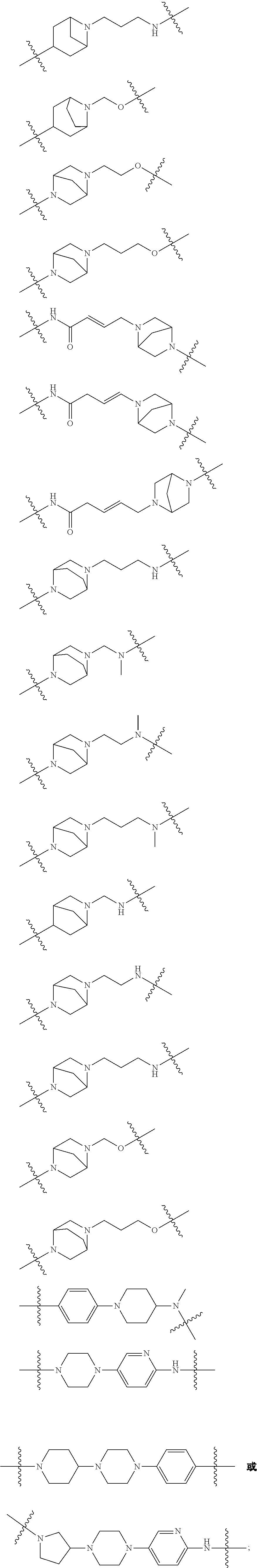

wherein the groups are optionally substituted with a substituent selected from the group consisting of deuterium, C₁₋₆ alkyl, deuterated C₁₋₆ alkyl, halogenated C₁₋₆ alkyl, C₁₋₆ alkoxy, halogen, amino, hydroxy, mercapto, cyano, carboxyl, C₂₋₆ alkynyl, or C₂₋₆ alkenyl. In the present disclosure, unless otherwise specified, any one end of the above-mentioned groups may be connected to R_{w} of the compound of Formula (I), while the other end is connected to R_{c} of the PBM.

In some embodiments, the compound of Formula (I) is also represented by Formula (I-1), Formula (I-2), Formula (I-3), Formula (I-4), Formula (I-5-1), Formula (I-5-2), Formula (I-5-3), Formula (I-5-4), Formula (I-6), Formula (I-7), Formula (I-8), Formula (I-9), Formula (I-10-1), Formula (I-10-2), Formula (I-11), Formula (I-12), Formula (I-13-1), Formula (I-13-2), Formula (I-14), Formula (I-15), Formula (I-16), Formula (I-17), Formula (I-18-1), Formula (I-18-2), Formula (I-19), Formula (I-20), Formula (I-21), Formula (I-22), Formula (I-23), Formula (I-24), Formula (I-25), Formula (I-26), Formula (I-27-1), Formula (I-27-2), Formula (I-28), Formula (I-29), Formula (I-30), Formula (I-31), Formula (I-32), or Formula (I-33):

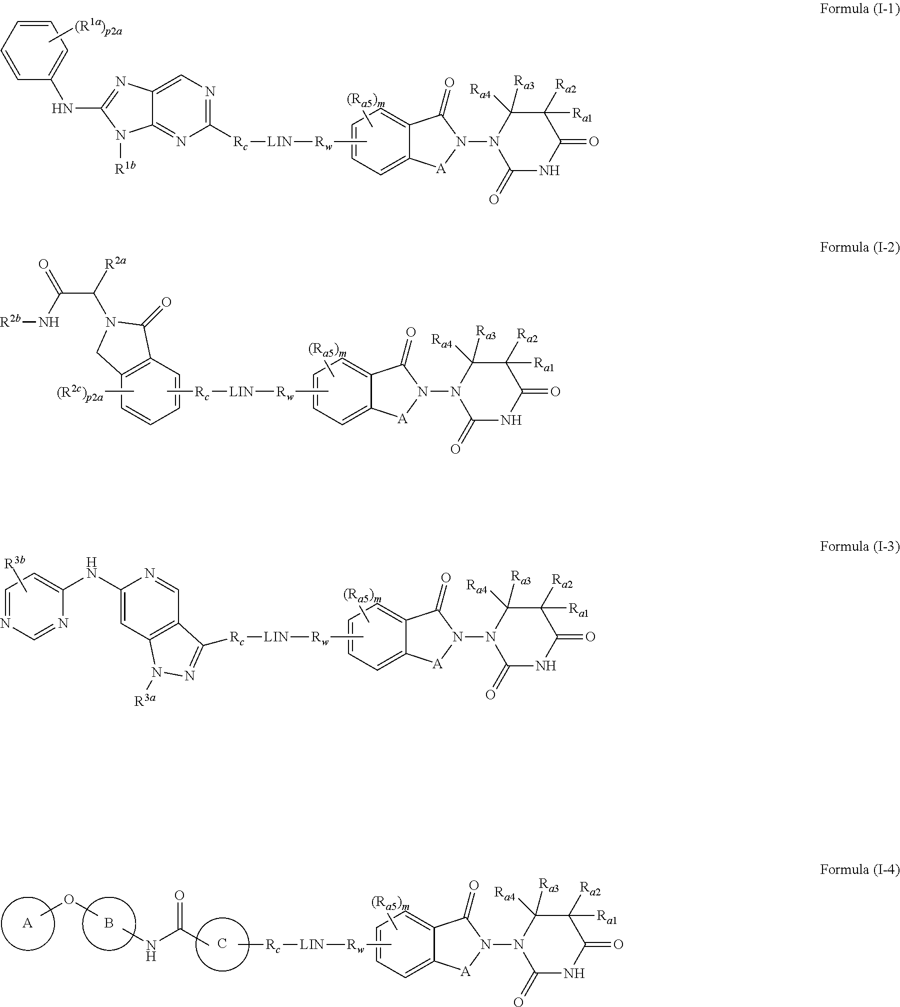

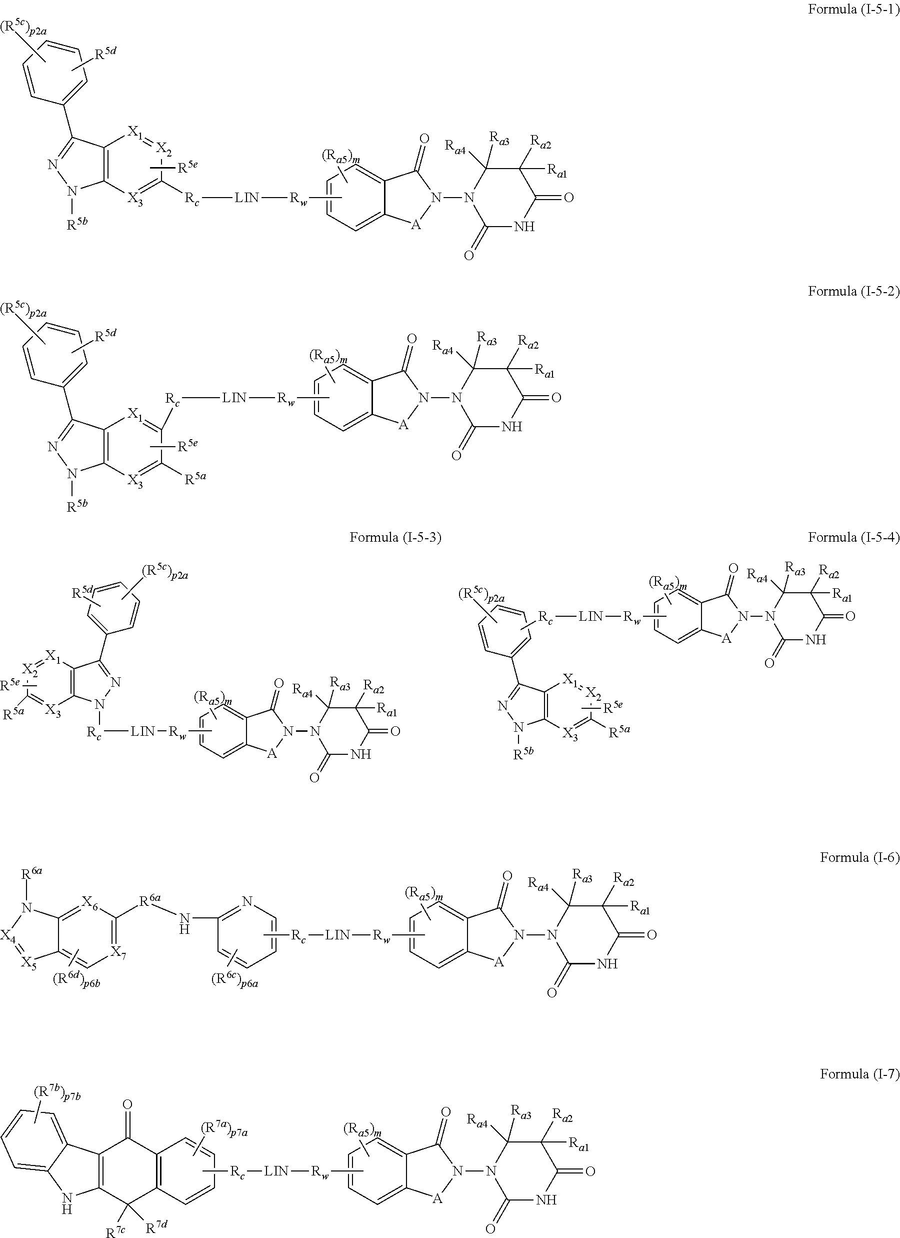

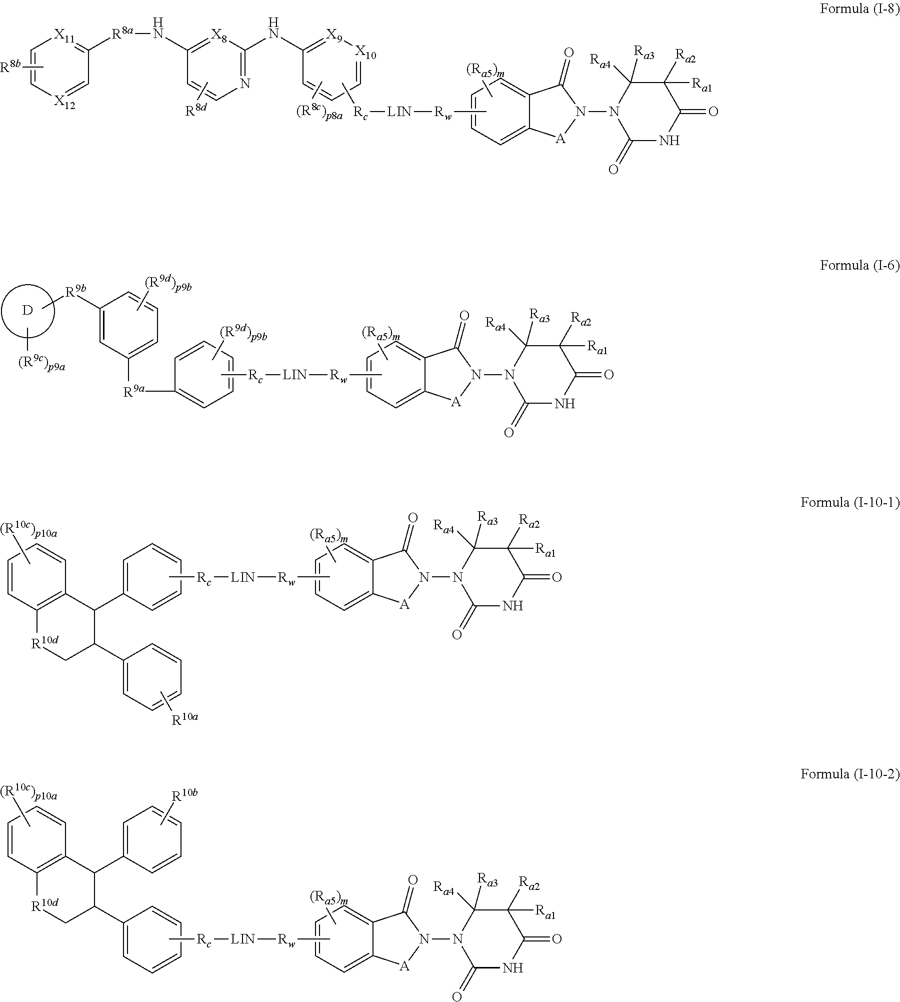

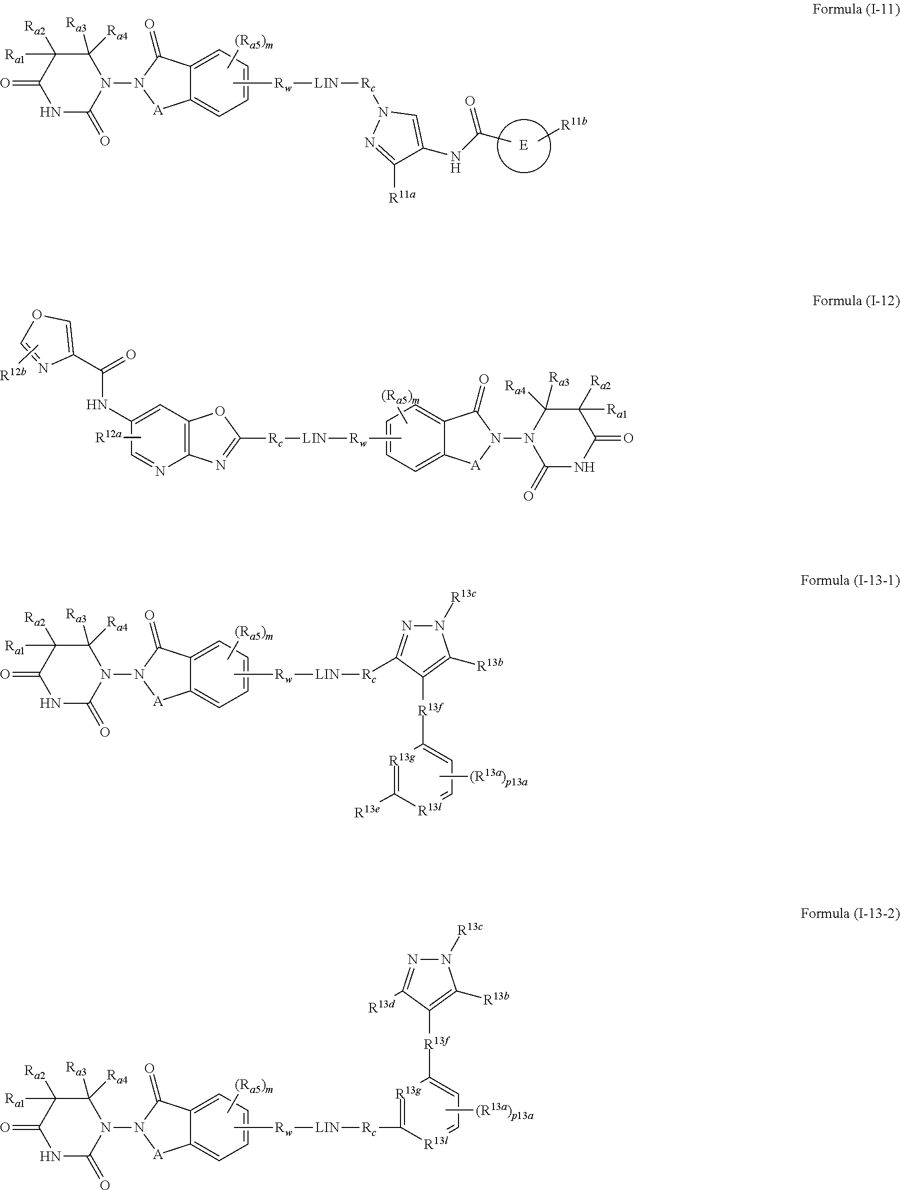

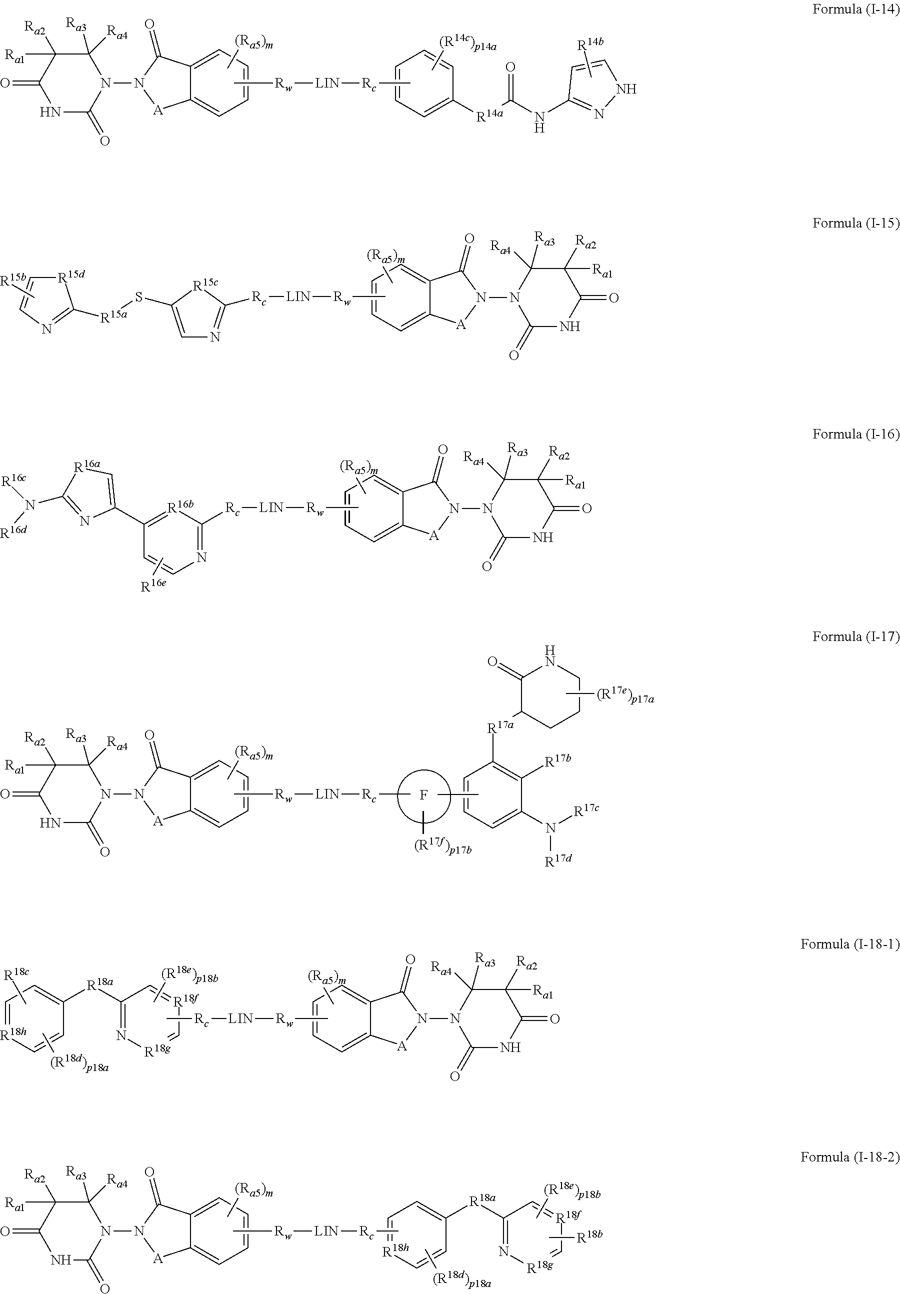

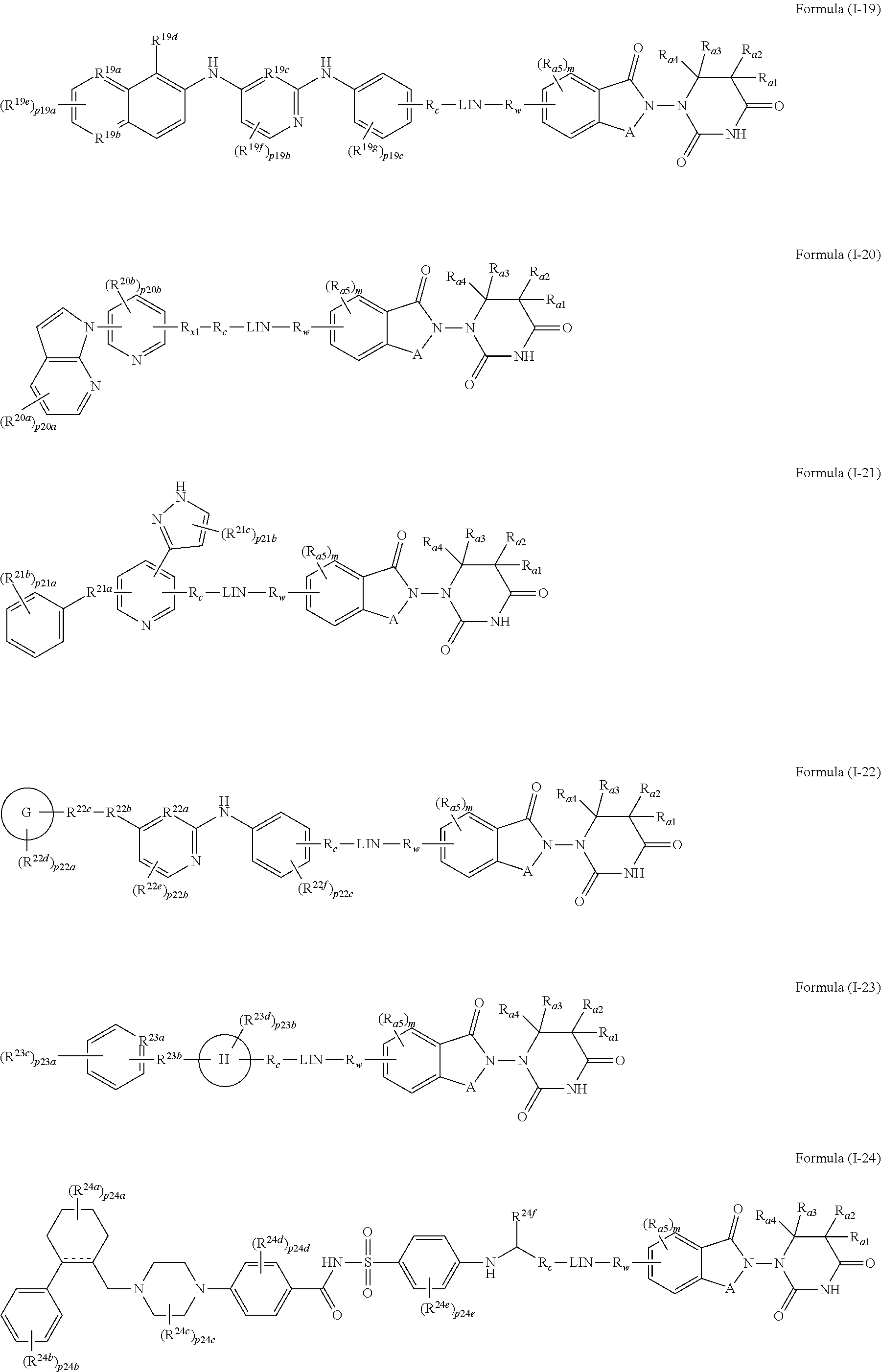

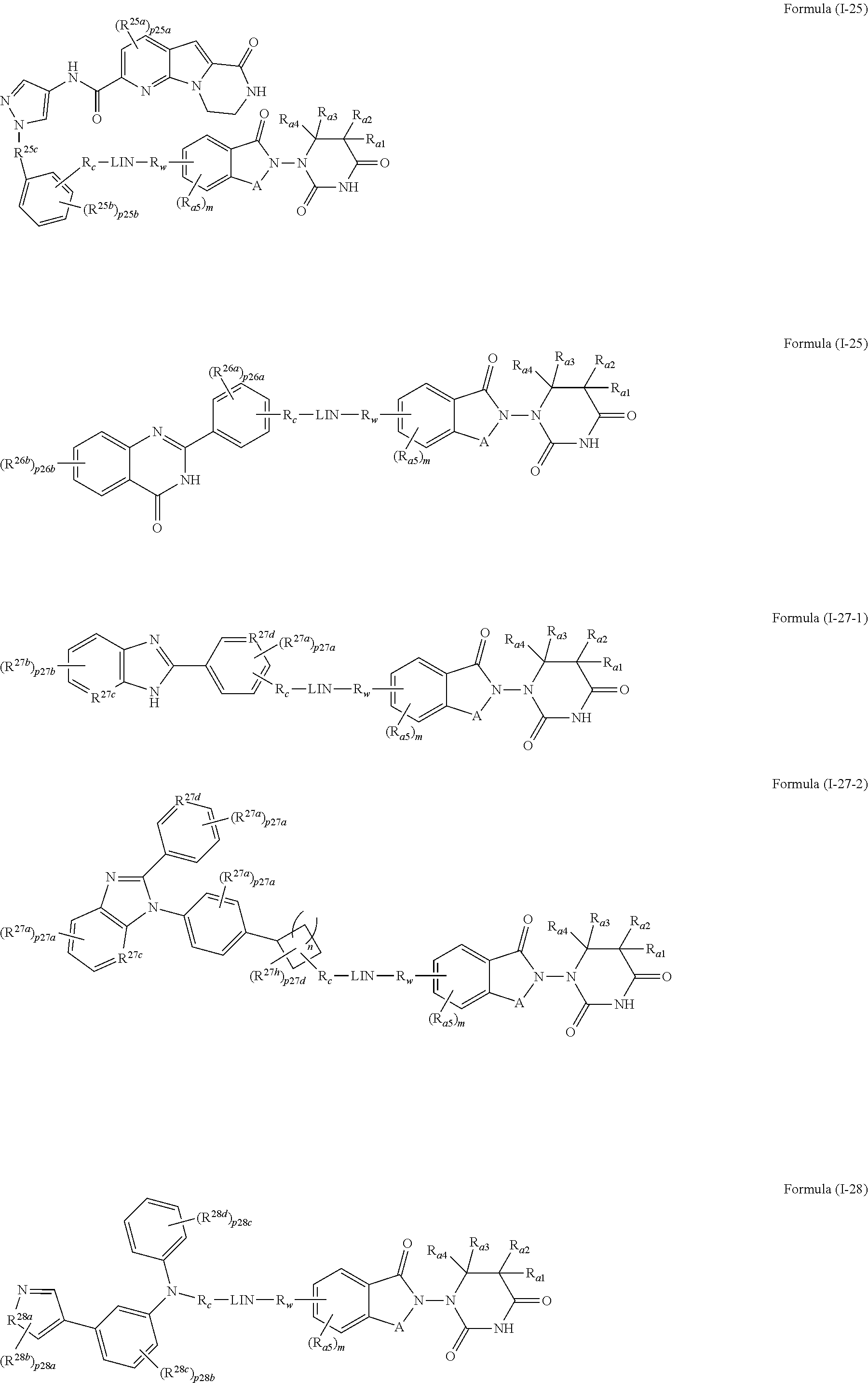

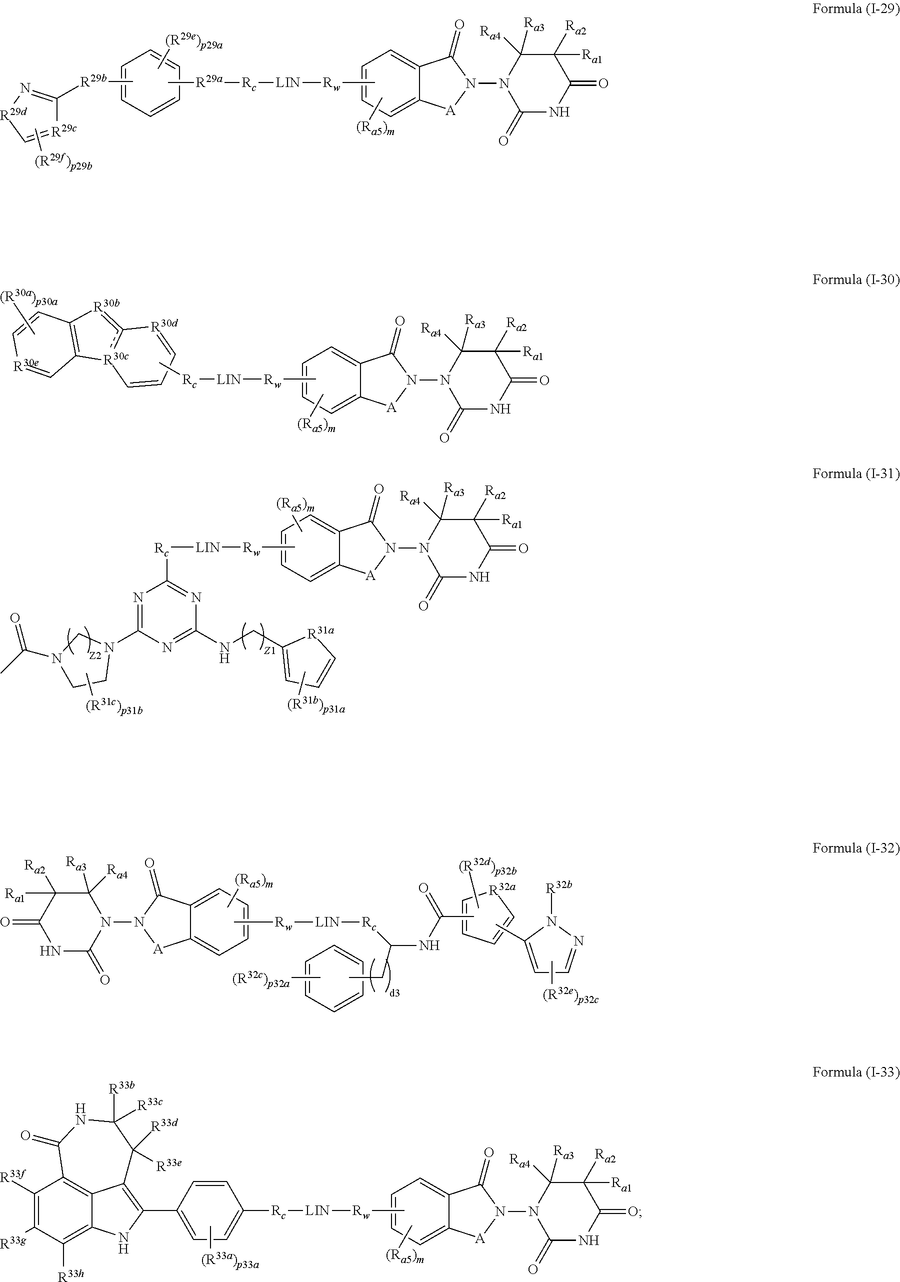

wherein A, Rₐ₁, Rₐ₂, Rₐ₃, Rₐ₄, (Rₐ₅)ₘ, R_{w}, LIN, R_{c}, (R^{1a})ₚ₁ₐ, R^{1b}, R^{2a}, R^{2b}, (R^{2c})ₚ₂ₐ, R^{3a}, R^{3b}, ring A, ring B, ring C, R^{5a}, R^{5b}, (R^{5c})ₚ₅ₐ, R^{5d}, R^{5e}, X₁, X₂, X₃, R^{6a}, R^{6b}, (R^{6c})ₚ₆ₐ, (R^{6d})_{p6b}, X₄, X₅, X₆, X₇, (R^{7a})ₚ₇ₐ, (R^{7b})_{p7b}, R^{7c}, R^{7d}, R^{8a}, R^{8b}, (R^{8c})ₚ₈ₐ, R^{8d}, X₈, X₉, X₁₀, X₁₁, X₁₂, R^{9a}, R^{9b}, (R^{9c})ₚ₉ₐ, (R^{9d})_{p9b}, R^{10a}, R^{10b}, (R^{10c})ₚ₁₀ₐ, R^{10d}, R^{11a}, R^{11b}, ring E, R^{12a}, R^{12b}, (R^{13a})ₚ₁₃ₐ, R^{13b}, R^{13c}, R^{13d}, R^{13e}, R^{13f}, R^{13g}, R¹³ⁱ, R₁₄ₐ, R^{14b}, (R^{14c})ₚ₁₄ₐ, R^{15a}, R^{15b}, R^{15c}, R^{15d}, R^{16a}, R^{16b}, R^{16c}, R^{16d}, R^{16e}, R^{17a}, R^{17b}, R^{17c}, R^{17d}, (R^{17e})ₚ₁₇ₐ, (R^{17f})_{p17b}, ring F, R^{18a}, R^{18c}, (R^{18d})ₚ₁₈ₐ, (R^{18e})_{p18b}, R^{18f}, R^{18g}, R^{18h}, R^{19a}, R^{19b}, R^{19c}, R^{19d}, (R^{19e})ₚ₁₉ₐ, (R^{19f})_{p19b}, (R^{19g})_{p19c}, (R^{20a})ₚ₂₀ₐ, (R^{20b})_{p20b}, Rₓ₁, R₂₁ₐ, (R^{21b})ₚ₂₁ₐ, (R_{21c})_{p21b}, R^{22a}, R^{22b}, R^{22c}, (R^{22d})ₚ₂₂ₐ, (R^{22e})_{p22b}, (R^{22f})_{p22c}, ring G, R^{23a}, R^{23b}, (R^{23c})ₚ₂₃ₐ, (R^{23d})_{p23b}, ring H, (R^{24a})ₚ₂₄ₐ, (R^{24b})_{p24b}, (R^{24c})_{p24c}, (R^{24d})_{p24d}, (R^{24e})ₚ₂₄ₑ, R^{24f}, (R^{25a})ₚ₂₅ₐ, (R^{25b})_{p25b}, R^{25c}, (R^{26a})ₚ₂₆ₐ, (R^{26b})_{p26b}, (R^{27a})ₚ₂₇ₐ, (R^{27b})_{p27b}, R^{27c}, R^{27d}, (R^{27g})_{p27c}, (R^{30a}) n, (R^{27h})ₚ₂₇ₐ, R^{28a}, (R^{28b})ₚ₂₈ₐ, (R^{28c})_{p28b}, (R^{28d})_{p28c}, R^{29a}, R^{29b}, R^{29c}, R^{29d}, (R^{29e})ₚ₂₉ₐ, (R^{29f})_{p29b}, (R^{30a})ₚ₃₀ₐ, [symbol] fragment

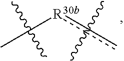

fragment

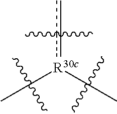

R^{30d}, R^{30e}, R^{31a}, (R^{31b})ₚ₃₁ₐ, (R^{31c})_{p31b}, Z1, Z2, R^{32a}, R^{32b}, (R^{32c})ₚ₃₂ₐ, (R^{32d})_{p32b}, (R^{32e})_{p32c}, Z3, (R^{33a})ₚ₃₃ₐ, R^{33b}, R^{33c}, R^{33d}, R^{33e}, R^{33f}, R^{33g}, and R^{33h} are as defined in the compound of Formula (I) and each sub-embodiments thereof.

In some embodiments, the compounds of Formula (I) of the present invention and their salts (including pharmaceutically acceptable salts, such as hydrochloride, etc.), prodrugs, solvates, isotopically enriched analogs, polymorphs, stereoisomers (including enantiomers and diastereomers), or mixture of stereoisomers thereof in Table 1 below are provided.

TABLE 1
The compounds of the present disclosure

| Compound | | |
|---|---|---|
| No. | Structure of compound | Compound′s name |
| GT- 0507 5 | 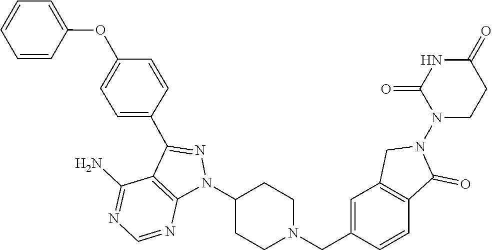 [SMILES: Nc1ncnc2c1c(-c1ccc(Oc3ccccc3)cc1)nn2C1CCN(Cc2ccc3c(c2)CN(N2CCC(=O)NC2=O)C3=O)CC1] | 1-(5-((4-(4-amino-3-(4-phenoxyphenyl)-1H- pyrazolo[3,4-d]pyrimidin-1-yl)piperidin-1- yl)methyl)-1-oxoisoindolin-2- yl)dihydropyrimidine-2,4(1H,3H)-dione |
| GT- 0583 6 | 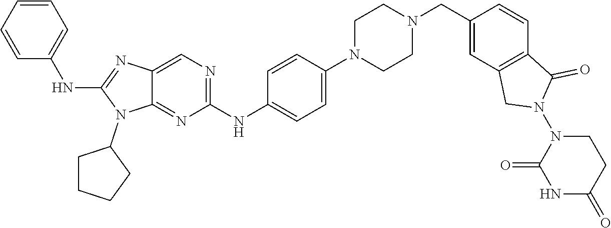 [SMILES: O=C1CCN(N2Cc3cc(CN4CCN(c5ccc(Nc6ncc7nc(Nc8ccccc8)n(C8CCCC8)c7n6)cc5)CC4)ccc3C2=O)C(=O)N1] | 1-(5-((4-(4-((9-cyclopentyl-8-(phenylamino)- 9H-purin-2-yl)amino)phenyl)piperazin-1- yl)methyl)-1-oxoisoindolin-2- yl)dihydropyrimidine-2,4(1H,3H)-dione |
| GT- 0584 2 | 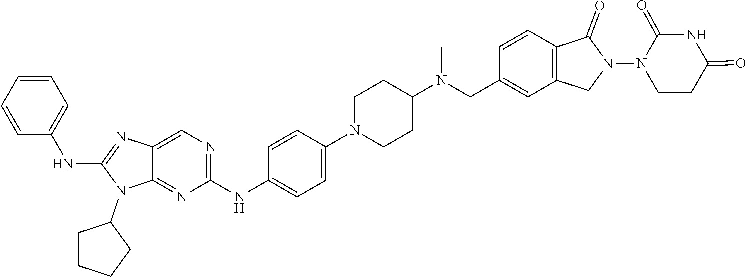 [SMILES: CN(Cc1ccc2c(c1)CN(N1CCC(=O)NC1=O)C2=O)C1CCN(c2ccc(Nc3ncc4nc(Nc5ccccc5)n(C5CCCC5)c4n3)cc2)CC1] | 1-(5-(((1-(4-((9-cyclopentyl-8-(phenylamino)- 9H-purin-2-yl)amino)phenyl)piperidin-4- yl)(methyl)amino)methyl)-1-oxoisoindolin-2- yl)dihydropyrimidine-2,4(1H,3H)-dione |
| GT- 0591 4 | 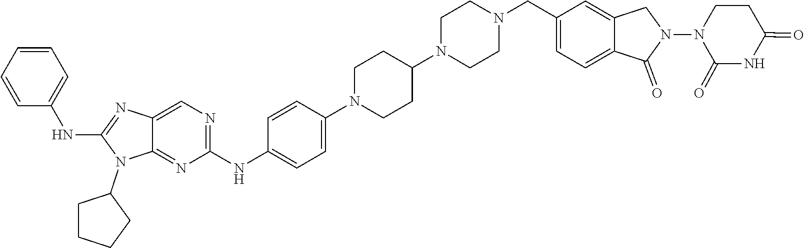 [SMILES: O=C1CCN(N2Cc3cc(CN4CCN(C5CCN(c6ccc(Nc7ncc8nc(Nc9ccccc9)n(C9CCCC9)c8n7)cc6)CC5)CC4)ccc3C2=O)C(=O)N1] | 1-(5-((4-(1-(4-((9-cyclopentyl-8- (phenylamino)-9H-purin-2- yl)amino)phenyl)piperidin-4-yl)piperazin-1- yl)methyl)-1-oxoisoindolin-2- yl)dihydropyrimidine-2,4(1H,3H)-dione |
| GT- 0594 6 | 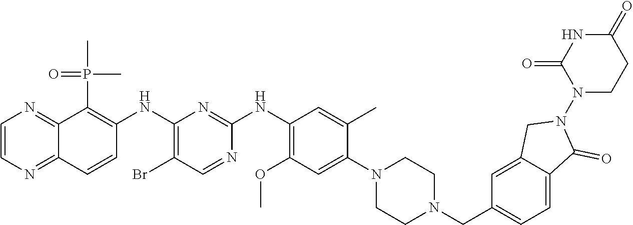 [SMILES: COc1cc(N2CCN(Cc3ccc4c(c3)CN(N3CCC(=O)NC3=O)C4=O)CC2)c(C)cc1Nc1ncc(Br)c(Nc2ccc3nccnc3c2P(C)(C)=O)n1] | 1-(5-((4-(4-((5-bromo-4-((5- (dimethylphosphoryl)quinoxalin-6- yl)amino)pyrimidin-2-yl)amino)-5-methoxy- 2-methylphenyl)piperazin-1-yl)methyl)-1- oxoisoindolin-2-yl)dihydropyrimidine- 2,4(1H,3H)-dione |
| GT- 0594 8 | 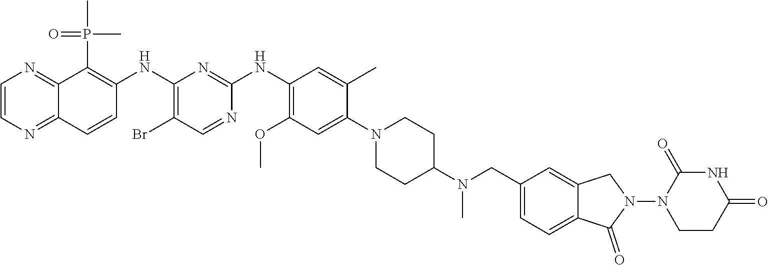 [SMILES: COc1cc(N2CCC(N(C)Cc3ccc4c(c3)CN(N3CCC(=O)NC3=O)C4=O)CC2)c(C)cc1Nc1ncc(Br)c(Nc2ccc3nccnc3c2P(C)(C)=O)n1] | 1-(5-(((1-(4-((5-bromo-4-((5- (dimethylphosphoryl)quinoxalin-6- yl)amino)pyrimidin-2-yl)amino)-5-methoxy- 2-methylphenyl)piperidin-4- yl)(methyl)amino)methyl)-1-oxoisoindolin-2- yl)dihydropyrimidine-2,4(1H,3H)-dione |
| GT- 0599 6 | 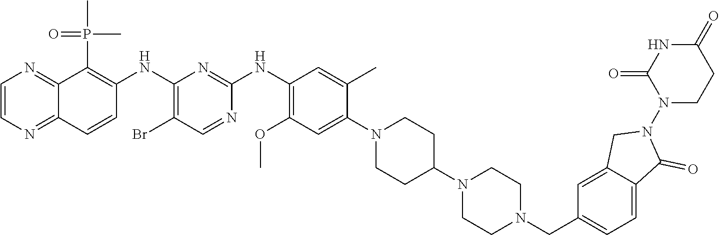 [SMILES: COc1cc(N2CCC(N3CCN(Cc4ccc5c(c4)CN(N4CCC(=O)NC4=O)C5=O)CC3)CC2)c(C)cc1Nc1ncc(Br)c(Nc2ccc3nccnc3c2P(C)(C)=O)n1] | 1-(5-((4-(1-(4-((5-bromo-4-((5- (dimethylphosphoryl)quinoxalin-6- yl)amino)pyrimidin-2-yl)amino)-5-methoxy- 2-methylphenyl)piperidin-4-yl)piperazin-1- yl)methyl)-1-oxoisoindolin-2- yl)dihydropyrimidine-2,4(1H,3H)-dione |
| | 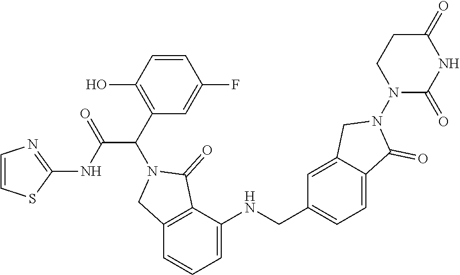 [SMILES: O=C1CCN(N2Cc3cc(CNc4cccc5c4C(=O)N(C(C(=O)Nc4nccs4)c4cc(F)ccc4O)C5)ccc3C2=O)C(=O)N1] | 2-(7-(((2-(2,4-dioxotetrahydropyrimidin- 1(2H)-yl)-1-oxoisoindolin-5- yl)methyl)amino)-1-oxoisoindolin-2-yl)-2-(5- fluoro-2-hydroxyphenyl)-N-(thiazol-2- yl)acetamide |
| | 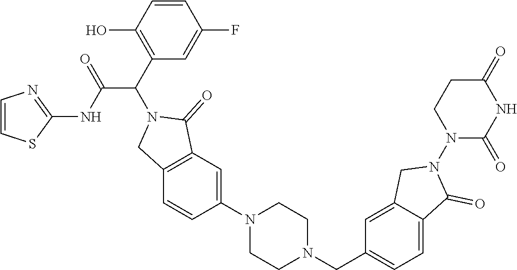 [SMILES: O=C1CCN(N2Cc3cc(CN4CCN(c5ccc6c(c5)C(=O)N(C(C(=O)Nc5nccs5)c5cc(F)ccc5O)C6)CC4)ccc3C2=O)C(=O)N1] | 2-(6-(4-((2-(2,4-dioxotetrahydropyrimidin- 1(2H)-yl)-1-oxoisoindolin-5- yl)methyl)piperazin-1-yl)-1-oxoisoindolin-2- yl)-2-(5-fluoro-2-hydroxyphenyl)-N-(thiazol- 2-yl)acetamide |
| | 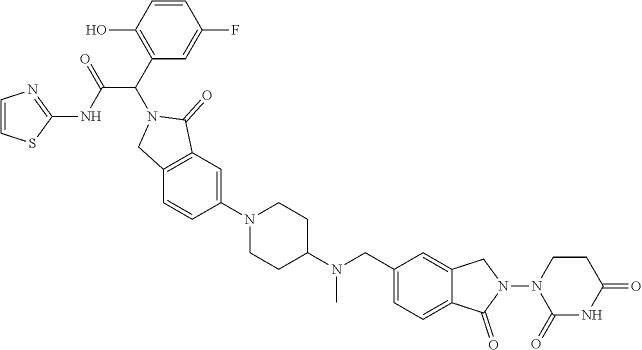 [SMILES: CN(Cc1ccc2c(c1)CN(N1CCC(=O)NC1=O)C2=O)C1CCN(c2ccc3c(c2)C(=O)N(C(C(=O)Nc2nccs2)c2cc(F)ccc2O)C3)CC1] | 2-(6-(4-(((2-(2,4-dioxotetrahydropyrimidin- 1(2H)-yl)-1-oxoisoindolin-5- yl)methyl)(methyl)amino)piperidin-1-yl)-1- oxoisoindolin-2-yl)-2-(5-fluoro-2- hydroxyphenyl)-N-(thiazol-2-yl)acetamide |
| | 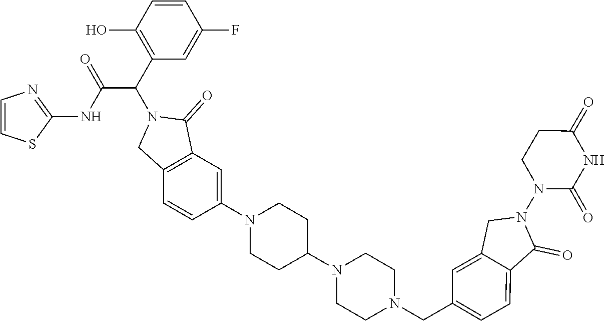 [SMILES: O=C1CCN(N2Cc3cc(CN4CCN(C5CCN(c6ccc7c(c6)C(=O)N(C(C(=O)Nc6nccs6)c6cc(F)ccc6O)C7)CC5)CC4)ccc3C2=O)C(=O)N1] | 2-(6-(4-(4-((2-(2,4-dioxotetrahydropyrimidin- 1(2H)-yl)-1-oxoisoindolin-5- yl)methyl)piperazin-1-yl)piperidin-1-yl)-1- oxoisoindolin-2-yl)-2-(5-fluoro-2- hydroxyphenyl)-N-(thiazol-2-yl)acetamide |
| | 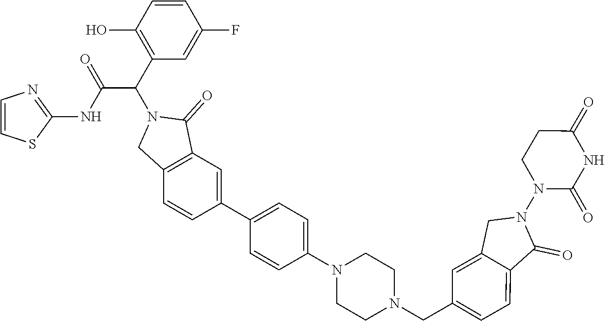 [SMILES: O=C1CCN(N2Cc3cc(CN4CCN(c5ccc(-c6ccc7c(c6)C(=O)N(C(C(=O)Nc6nccs6)c6cc(F)ccc6O)C7)cc5)CC4)ccc3C2=O)C(=O)N1] | 2-(6-(4-(4-((2-(2,4-dioxotetrahydropyrimidin- 1(2H)-yl)-1-oxoisoindolin-5- yl)methyl)piperazin-1-yl)phenyl)-1- oxoisoindolin-2-yl)-2-(5-fluoro-2- hydroxyphenyl)-N-(thiazol-2-yl)acetamide |
| | 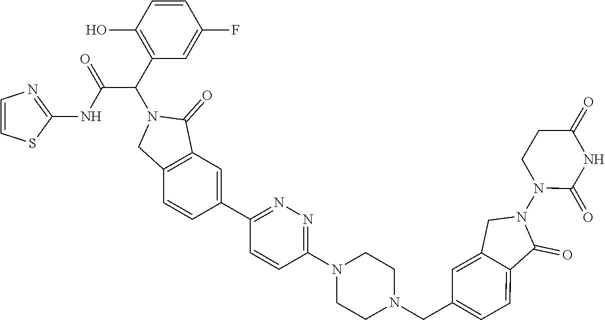 [SMILES: O=C1CCN(N2Cc3cc(CN4CCN(c5ccc(-c6ccc7c(c6)C(=O)N(C(C(=O)Nc6nccs6)c6cc(F)ccc6O)C7)nn5)CC4)ccc3C2=O)C(=O)N1] | 2-(6-(6-(4-((2-(2,4-dioxotetrahydropyrimidin- 1(2H)-yl)-1-oxoisoindolin-5- yl)methyl)piperazin-1-yl)pyridazin-3-yl)-1- oxoisoindolin-2-yl)-2-(5-fluoro-2- hydroxyphenyl)-N-(thiazol-2-yl)acetamide |
| GT- 0615 9 | 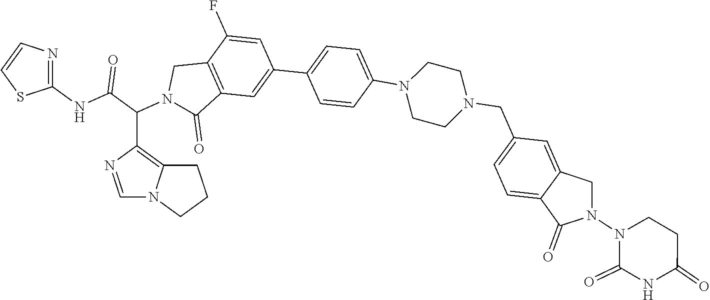 [SMILES: O=C1CCN(N2Cc3cc(CN4CCN(c5ccc(-c6cc(F)c7c(c6)C(=O)N(C(C(=O)Nc6nccs6)c6ncn8c6CCC8)C7)cc5)CC4)ccc3C2=O)C(=O)N1] | 2-(6,7-dihydro-5H-pyrrolo[1,2-c]imidazol-1- yl)-2-(6-(4-(4-((2-(2,4- dioxotetrahydropyrimidin-1(2H)-yl)-1- oxoisoindolin-5-yl)methyl)piperazin-1- yl)phenyl)-4-fluoro-1-oxoisoindolin-2-yl)-N- (thiazol-2-yl)acetamide |
| GT- 0511 4 | 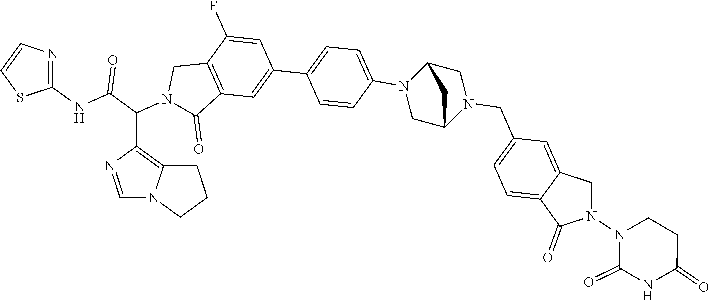 [SMILES: O=C1CCN(N2Cc3cc(CN4C[C@@H]5C[C@H]4CN5c4ccc(-c5cc(F)c6c(c5)C(=O)N(C(C(=O)Nc5nccs5)c5ncn7c5CCC7)C6)cc4)ccc3C2=O)C(=O)N1] | 2-(6,7-dihydro-5H-pyrrolo[1,2-c]imidazol-1- yl)-2-(6-(4-((1S,4S)-5-((2-(2,4- dioxotetrahydropyrimidin-1(2H)-yl)-1- oxoisoindolin-5-yl)methyl)-2,5- diazabicyclo[2.2.1]heptan-2-yl)phenyl)-4- fluoro-1-oxoisoindolin-2-yl)-N-(thiazol-2- yl)acetamide |
| GT- 0617 2 | 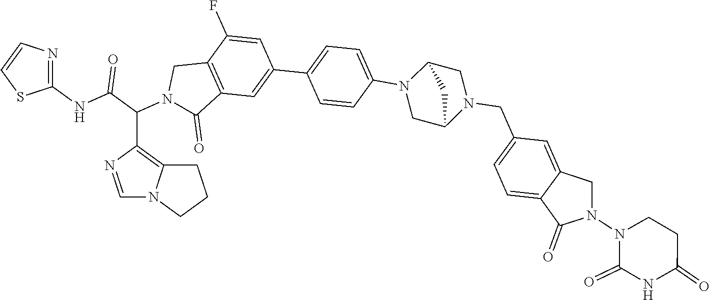 [SMILES: O=C1CCN(N2Cc3cc(CN4C[C@H]5C[C@@H]4CN5c4ccc(-c5cc(F)c6c(c5)C(=O)N(C(C(=O)Nc5nccs5)c5ncn7c5CCC7)C6)cc4)ccc3C2=O)C(=O)N1] | 2-(6,7-dihydro-5H-pyrrolo[1,2-c]imidazol-1- yl)-2-(6-(4-((1R,4R)-5-((2-(2,4- dioxotetrahydropyrimidin-1(2H)-yl)-1- oxoisoindolin-5-yl)methyl)-2,5- diazabicyclo[2.2.1]heptan-2-yl)phenyl)-4- fluoro-1-oxoisoindolin-2-yl)-N-(thiazol-2- yl)acetamide |
| GT- 0625 0 | 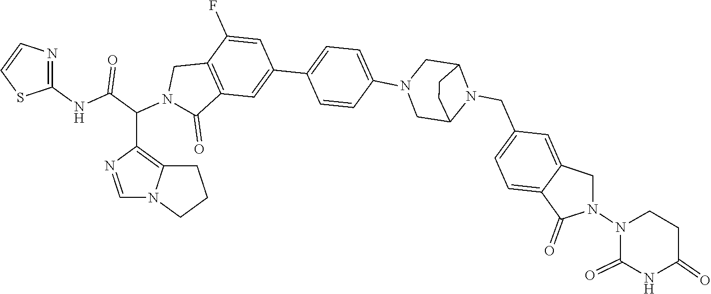 [SMILES: O=C1CCN(N2Cc3cc(CN4C5CCC4CN(c4ccc(-c6cc(F)c7c(c6)C(=O)N(C(C(=O)Nc6nccs6)c6ncn8c6CCC8)C7)cc4)C5)ccc3C2=O)C(=O)N1] | 2-(6,7-dihydro-5H-pyrrolo[1,2-c]imidazol-1- yl)-2-(6-(4-(8-((2-(2,4- dioxotetrahydropyrimidin-1(2H)-yl)-1- oxoisoindolin-5-yl)methyl)-3,8- diazabicyclo[3.2.1]octan-3-yl)phenyl)-4- fluoro-1-oxoisoindolin-2-yl)-N-(thiazol-2- yl)acetamide |
| GT- 0509 9 | 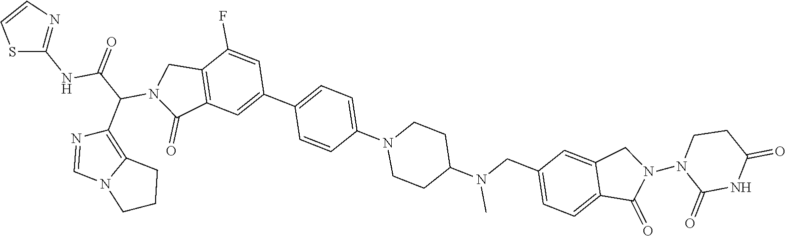 [SMILES: CN(Cc1ccc2c(c1)CN(N1CCC(=O)NC1=O)C2=O)C1CCN(c2ccc(-c3cc(F)c4c(c3)C(=O)N(C(C(=O)Nc3nccs3)c3ncn5c3CCC5)C4)cc2)CC1] | 2-(6,7-dihydro-5H-pyrrolo[1,2-c]imidazol-1- yl)-2-(6-(4-(4-(((2-(2,4- dioxotetrahydropyrimidin-1(2H)-yl)-1- oxoisoindolin-5- yl)methyl)(methyl)amino)piperidin-1- yl)phenyl)-4-fluoro-1-oxoisoindolin-2-yl)-N- (thiazol-2-yl)acetamide |
| GT- 0624 1 | 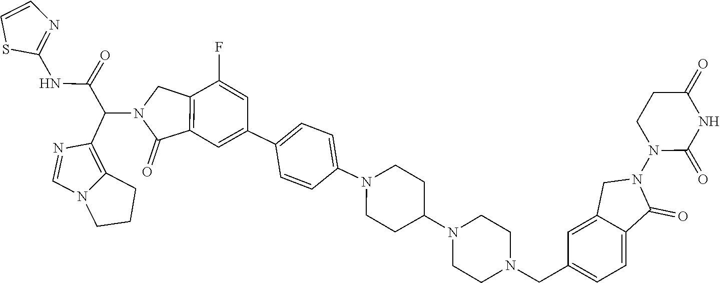 [SMILES: O=C1CCN(N2Cc3cc(CN4CCN(C5CCN(c6ccc(-c7cc(F)c8c(c7)C(=O)N(C(C(=O)Nc7nccs7)c7ncn9c7CCC9)C8)cc6)CC5)CC4)ccc3C2=O)C(=O)N1] | 2-(6,7-dihydro-5H-pyrrolo[1,2-c]imidazol-1- yl)-2-(6-(4-(4-(4-((2-(2,4- dioxotetrahydropyrimidin-1(2H)-yl)-1- oxoisoindolin-5-yl)methyl)piperazin-1- yl)piperidin-1-yl)phenyl)-4-fluoro-1- oxoisoindolin-2-yl)-N-(thiazol-2- yl)acetamide |
| GT- 0631 5 | 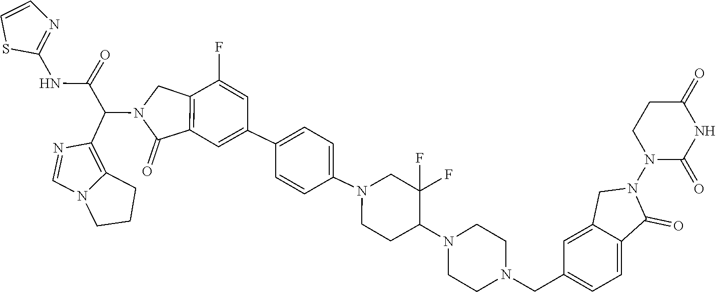 [SMILES: O=C1CCN(N2Cc3cc(CN4CCN(C5CCN(c6ccc(-c7cc(F)c8c(c7)C(=O)N(C(C(=O)Nc7nccs7)c7ncn9c7CCC9)C8)cc6)CC5(F)F)CC4)ccc3C2=O)C(=O)N1] | 2-(6,7-dihydro-5H-pyrrolo[1,2-c]imidazol-1- yl)-2-(6-(4-(4-(4-((2-(2,4- dioxotetrahydropyrimidin-1(2H)-yl)-1- oxoisoindolin-5-yl)methyl)piperazin-1-yl)- 3,3-difluoropiperidin-1-yl)phenyl)-4-fluoro-1- oxoisoindolin-2-yl)-N-(thiazol-2- yl)acetamide |
| GT- 0632 4 | 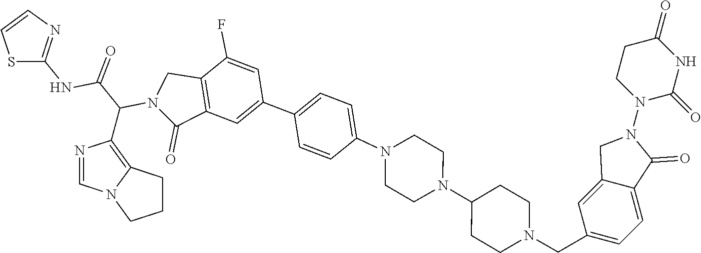 [SMILES: O=C1CCN(N2Cc3cc(CN4CCC(N5CCN(c6ccc(-c7cc(F)c8c(c7)C(=O)N(C(C(=O)Nc7nccs7)c7ncn9c7CCC9)C8)cc6)CC5)CC4)ccc3C2=O)C(=O)N1] | 2-(6,7-dihydro-5H-pyrrolo[1,2-c]imidazol-1- yl)-2-(6-(4-(4-(1-((2-(2,4- dioxotetrahydropyrimidin-1(2H)-yl)-1- oxoisoindolin-5-yl)methyl)piperidin-4- yl)piperazin-1-yl)phenyl)-4-fluoro-1- oxoisoindolin-2-yl)-N-(thiazol-2- yl)acetamide |
| | 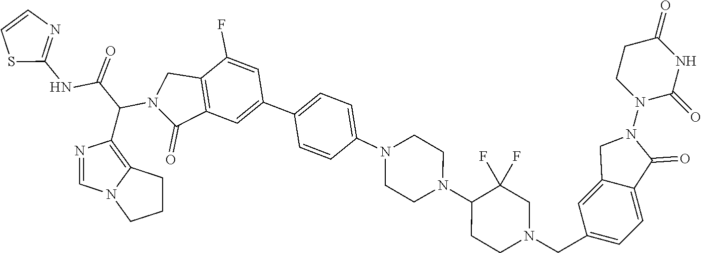 [SMILES: O=C1CCN(N2Cc3cc(CN4CCC(N5CCN(c6ccc(-c7cc(F)c8c(c7)C(=O)N(C(C(=O)Nc7nccs7)c7ncn9c7CCC9)C8)cc6)CC5)C(F)(F)C4)ccc3C2=O)C(=O)N1] | 2-(6,7-dihydro-5H-pyrrolo[1,2-c]imidazol-1- yl)-2-(6-(4-(4-(1-((2-(2,4- dioxotetrahydropyrimidin-1(2H)-yl)-1- oxoisoindolin-5-yl)methyl)-3,3- difluoropiperidin-4-yl)piperazin-1-yl)phenyl)- 4-fluoro-1-oxoisoindolin-2-yl)-N-(thiazol-2- yl)acetamide |
| GT- 0601 0 | 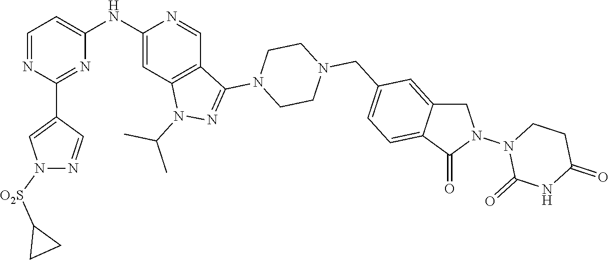 [SMILES: CC(C)n1nc(N2CCN(Cc3ccc4c(c3)CN(N3CCC(=O)NC3=O)C4=O)CC2)c2cnc(Nc3ccnc(-c4cnn(S(=O)(=O)C5CC5)c4)n3)cc21] | 1-(5-((4-(6-((2-(1-(cyclopropylsulfonyl)-1H- pyrazol-4-yl)pyrimidin-4-yl)amino)-1- isopropyl-1H-pyrazolo[4,3-c]pyridin-3- yl)piperazin-1-yl)methyl)-1-oxoisoindolin-2- yl)dihydropyrimidine-2,4(1H,3H)-dione |
| GT- 0601 9 | 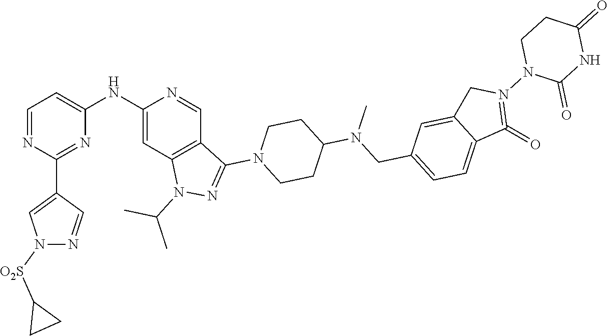 | 1-(5-(((1-(6-((2-(1-(cyclopropylsulfonyl)-1H- pyrazol-4-yl)pyrimidin-4-yl)amino)-1- isopropyl-1H-pyrazolo[4,3-c]pyridin-3- yl)piperidin-4-yl)(methyl)amino)methyl)-1- oxoisoindolin-2-yl)dihydropyrimidine- 2,4(1H,3H)-dione |
| GT- 0607 7 | 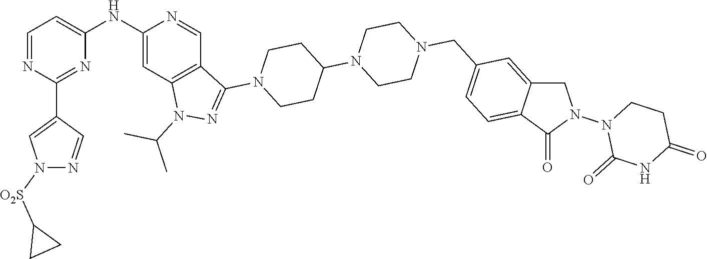 [SMILES: CC(C)n1nc(N2CCC(N3CCN(Cc4ccc5c(c4)CN(N4CCC(=O)NC4=O)C5=O)CC3)CC2)c2cnc(Nc3ccnc(-c4cnn(S(=O)(=O)C5CC5)c4)n3)cc21] | 1-(5-((4-(1-(6-((2-(1-(cyclopropylsulfonyl)- 1H-pyrazol-4-yl)pyrimidin-4-yl)amino)-1- isopropyl-1H-pyrazolo[4,3-c]pyridin-3- yl)piperidin-4-yl)piperazin-1-yl)methyl)-1- oxoisoindolin-2-yl)dihydropyrimidine- 2,4(1H,3H)-dione |
| GT- 0603 4 | 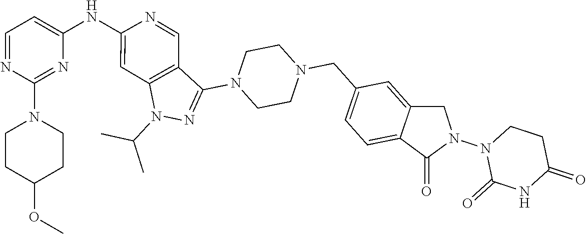 [SMILES: COC1CCN(c2nccc(Nc3cc4c(cn3)c(N3CCN(Cc5ccc6c(c5)CN(N5CCC(=O)NC5=O)C6=O)CC3)nn4C(C)C)n2)CC1] | 1-(5-((4-(1-isopropyl-6-((2-(4- methoxypiperidin-1-yl)pyrimidin-4- yl)amino)-1H-pyrazolo[4,3-c]pyridin-3- yl)piperazin-1-yl)methyl)-1-oxoisoindolin-2- yl)dihydropyrimidine-2,4(1H,3H)-dione |
| GT- 0610 9 | 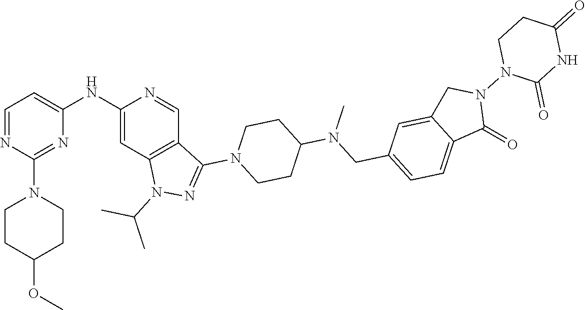 [SMILES: COC1CCN(c2nccc(Nc3cc4c(cn3)c(N3CCC(N(C)Cc5ccc6c(c5)CN(N5CCC(=O)NC5=O)C6=O)CC3)nn4C(C)C)n2)CC1] | 1-(5-(((1-(1-isopropyl-6-((2-(4- methoxypiperidin-1-yl)pyrimidin-4- yl)amino)-1H-pyrazolo[4,3-c]pyridin-3- yl)piperidin-4-yl)(methyl)amino)methyl)-1- oxoisoindolin-2-yl)dihydropyrimidine- 2,4(1H,3H)-dione |
| | 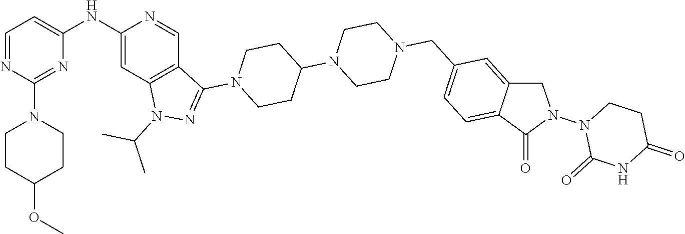 [SMILES: COC1CCN(c2nccc(Nc3cc4c(cn3)c(N3CCC(N5CCN(Cc6ccc7c(c6)CN(N6CCC(=O)NC6=O)C7=O)CC5)CC3)nn4C(C)C)n2)CC1] | 1-(5-((4-(1-(1-isopropyl-6-((2-(4- methoxypiperidin-1-yl)pyrimidin-4- yl)amino)-1H-pyrazolo[4,3-c]pyridin-3- yl)piperidin-4-yl)piperazin-1-yl)methyl)-1- oxoisoindolin-2-yl)dihydropyrimidine- 2,4(1H,3H)-dione |
| | 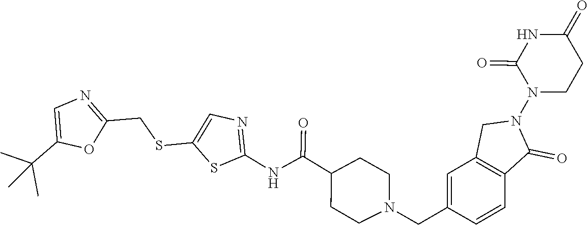 [SMILES: CC(C)(C)c1cnc(CSc2cnc(NC(=O)C3CCN(Cc4ccc5c(c4)CN(N4CCC(=O)NC4=O)C5=O)CC3)s2)o1] | N-(5-(((5-(tert-butyl)oxazol-2- yl)methyl)thio)thiazol-2-yl)-1-((2-(2,4- dioxotetrahydropyrimidin-1(2H)-yl)-1- oxoisoindolin-5-yl)methyl)piperidine-4- carboxamide |
| | 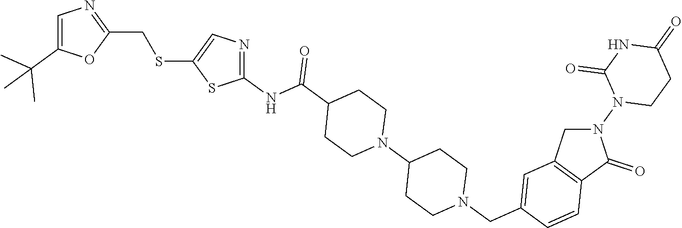 [SMILES: CC(C)(C)c1cnc(CSc2cnc(NC(=O)C3CCN(C4CCN(Cc5ccc6c(c5)CN(N5CCC(=O)NC5=O)C6=O)CC4)CC3)s2)o1] | N-(5-(((5-(tert-butyl)oxazol-2- yl)methyl)thio)thiazol-2-yl)-1′-((2-(2,4- dioxotetrahydropyrimidin-1(2H)-yl)-1- oxoisoindolin-5-yl)methyl)-[1,4′- bipiperidine]-4-carboxamide |
| | 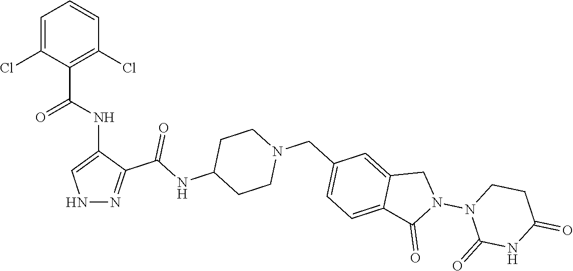 [SMILES: O=C1CCN(N2Cc3cc(CN4CCC(NC(=O)c5n[nH]cc5NC(=O)c5c(Cl)cccc5Cl)CC4)ccc3C2=O)C(=O)N1] | 4-(2,6-dichlorobenzamido)-N-(1-((2-(2,4- dioxotetrahydropyrimidin-1(2H)-yl)-1- oxoisoindolin-5-yl)methyl)piperidin-4-yl)- 1H-pyrazole-3-carboxamide |
| | 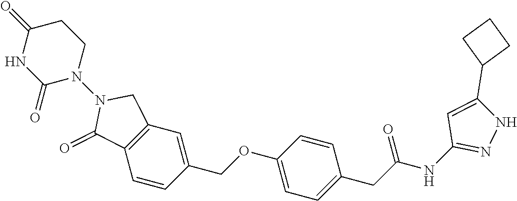 [SMILES: O=C1CCN(N2Cc3cc(COc4ccc(CC(=O)Nc5cc(C6CCC6)[nH]n5)cc4)ccc3C2=O)C(=O)N1] | N-(5-cyclobutyl-1H-pyrazol-3-yl)-2-(4-((2- (2,4-dioxotetrahydropyrimidin-1(2H)-yl)-1- oxoisoindolin-5- yl)methoxy)phenyl)acetamide |
| GT- 0821 2 | 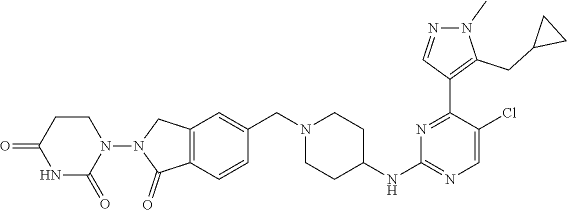 [SMILES: Cn1ncc(-c2nc(NC3CCN(Cc4ccc5c(c4)CN(N4CCC(=O)NC4=O)C5=O)CC3)ncc2Cl)c1CC1CC1] | 1-(5-((4-((5-chloro-4-(5-(cyclopropylmethyl)- 1-methyl-1H-pyrazol-4-yl)pyrimidin-2- yl)amino)piperidin-1-yl)methyl)-1- oxoisoindolin-2-yl)dihydropyrimidine- 2,4(1H,3H)-dione |
| GT- 0815 3 | 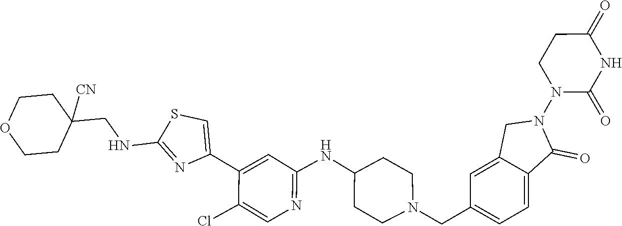 [SMILES: N#CC1(CNc2nc(-c3cc(NC4CCN(Cc5ccc6c(c5)CN(N5CCC(=O)NC5=O)C6=O)CC4)ncc3Cl)cs2)CCOCC1] | 4-(((4-(5-chloro-2-((1-((2-(2,4- dioxotetrahydropyrimidin-1(2H)-yl)-1- oxoisoindolin-5-yl)methyl)piperidin-4- yl)amino)pyridin-4-yl)thiazol-2- yl)amino)methyl)tetrahydro-2H-pyran-4- carbonitrile |
| GT- 0821 4 | 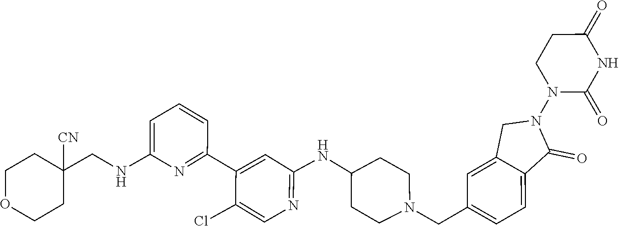 [SMILES: N#CC1(CNc2cccc(-c3cc(NC4CCN(Cc5ccc6c(c5)CN(N5CCC(=O)NC5=O)C6=O)CC4)ncc3Cl)n2)CCOCC1] | 4-(((5′-chloro-2′-((1-((2-(2,4- dioxotetrahydropyrimidin-1(2H)-yl)-1- oxoisoindolin-5-yl)methyl)piperidin-4- yl)amino)-[2,4′-bipyridin]-6- yl)amino)methyl)tetrahydro-2H-pyran-4- carbonitrile |
| | 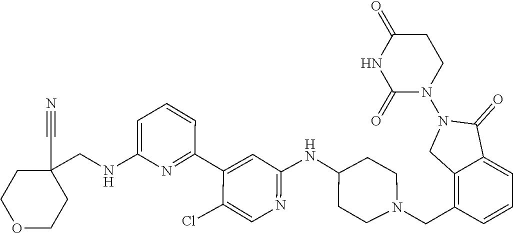 [SMILES: N#CC1(CNc2cccc(-c3cc(NC4CCN(Cc5cccc6c5CN(N5CCC(=O)NC5=O)C6=O)CC4)ncc3Cl)n2)CCOCC1] | 4-(((5′-chloro-2′-((1-((2-(2,4- dioxotetrahydropyrimidin-1(2H)-yl)-1- oxoisoindolin-4-yl)methyl)piperidin-4- yl)amino)-[2,4′-bipyridin]-6- yl)amino)methyl)tetrahydro-2H-pyran-4- carbonitrile |
| GT- 0822 2 | 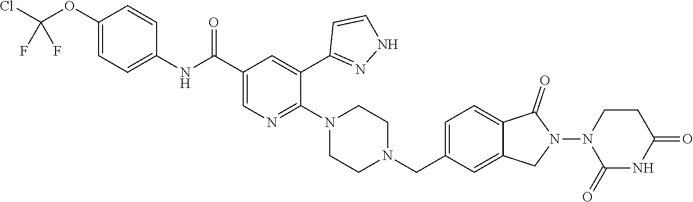 [SMILES: O=C1CCN(N2Cc3cc(CN4CCN(c5ncc(C(=O)Nc6ccc(OC(F)(F)Cl)cc6)cc5-c5cc[nH]n5)CC4)ccc3C2=O)C(=O)N1] | N-(4-(chlorodifluoromethoxy)phenyl)-6-(4- ((2-(2,4-dioxotetrahydropyrimidin-1(2H)-yl)- 1-oxoisoindolin-5-yl)methyl)piperazin-1-yl)- 5-(1H-pyrazol-3-yl)nicotinamide |
| | 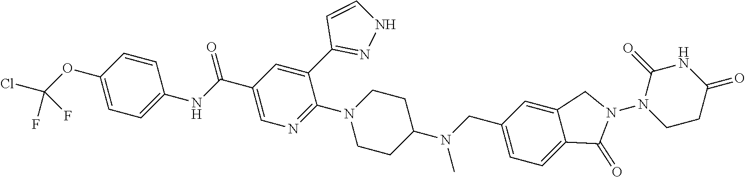 [SMILES: CN(Cc1ccc2c(c1)CN(N1CCC(=O)NC1=O)C2=O)C1CCN(c2ncc(C(=O)Nc3ccc(OC(F)(F)Cl)cc3)cc2-c2cc[nH]n2)CC1] | N-(4-(chlorodifluoromethoxy)phenyl)-6-(4- (((2-(2,4-dioxotetrahydropyrimidin-1(2H)- yl)-1-oxoisoindolin-5- yl)methyl)(methyl)amino)piperidin-1-yl)-5- (1H-pyrazol-3-yl)nicotinamide |
| GT- 0822 4 | 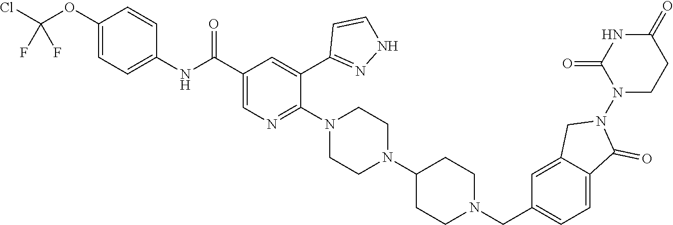 [SMILES: O=C1CCN(N2Cc3cc(CN4CCC(N5CCN(c6ncc(C(=O)Nc7ccc(OC(F)(F)Cl)cc7)cc6-c6cc[nH]n6)CC5)CC4)ccc3C2=O)C(=O)N1] | N-(4-(chlorodifluoromethoxy)phenyl)-6-(4- (1-((2-(2,4-dioxotetrahydropyrimidin-1(2H)- yl)-1-oxoisoindolin-5-yl)methyl)piperidin-4- yl)piperazin-1-yl)-5-(1H-pyrazol-3- yl)nicotinamide |
| GT- 0822 6 | 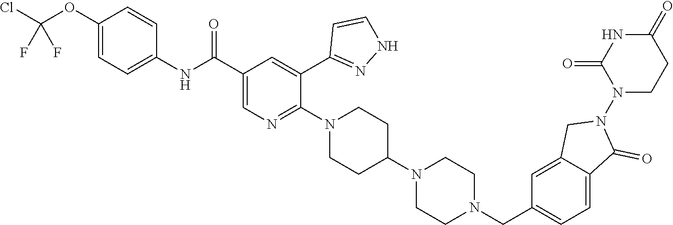 [SMILES: O=C1CCN(N2Cc3cc(CN4CCN(C5CCN(c6ncc(C(=O)Nc7ccc(OC(F)(F)Cl)cc7)cc6-c6cc[nH]n6)CC5)CC4)ccc3C2=O)C(=O)N1] | N-(4-(chlorodifluoromethoxy)phenyl)-6-(4- (4-((2-(2,4-dioxotetrahydropyrimidin-1(2H)- yl)-1-oxoisoindolin-5-yl)methyl)piperazin-1- yl)piperidin-1-yl)-5-(1H-pyrazol-3- yl)nicotinamide |
| GT- 0821 6 | 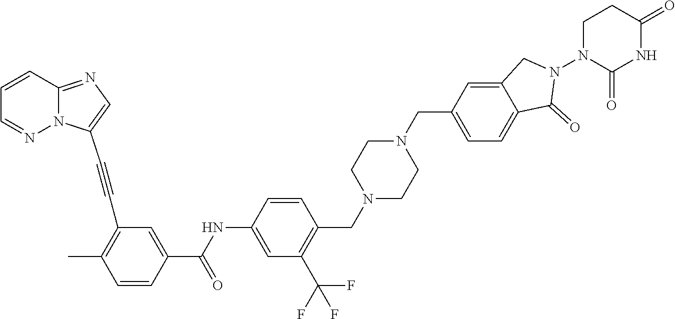 [SMILES: Cc1ccc(C(=O)Nc2ccc(CN3CCN(Cc4ccc5c(c4)CN(N4CCC(=O)NC4=O)C5=O)CC3)c(C(F)(F)F)c2)cc1C#Cc1cnc2cccnn12] | N-(4-((4-((2-(2,4-dioxotetrahydropyrimidin- 1(2H)-yl)-1-oxoisoindolin-5- yl)methyl)piperazin-1-yl)methyl)-3- (trifluoromethyl)phenyl)-3-(imidazo[1,2- b]pyridazin-3-ylethynyl)-4-methylbenzamide |
| GT- 0821 8 | 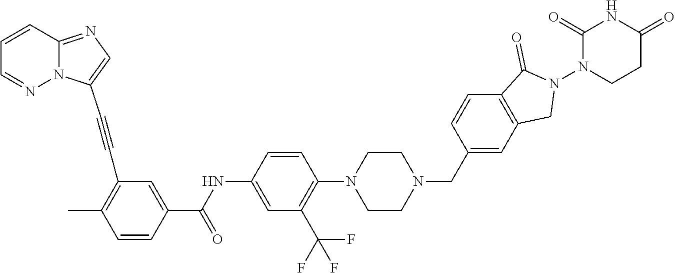 [SMILES: Cc1ccc(C(=O)Nc2ccc(N3CCN(Cc4ccc5c(c4)CN(N4CCC(=O)NC4=O)C5=O)CC3)c(C(F)(F)F)c2)cc1C#Cc1cnc2cccnn12] | N-(4-(4-((2-(2,4-dioxotetrahydropyrimidin- 1(2H)-yl)-1-oxoisoindolin-5- yl)methyl)piperazin-1-yl)-3- (trifluoromethyl)phenyl)-3-(imidazo[1,2- b\|pyridazin-3-ylethynyl)-4-methylbenzamide |
| | 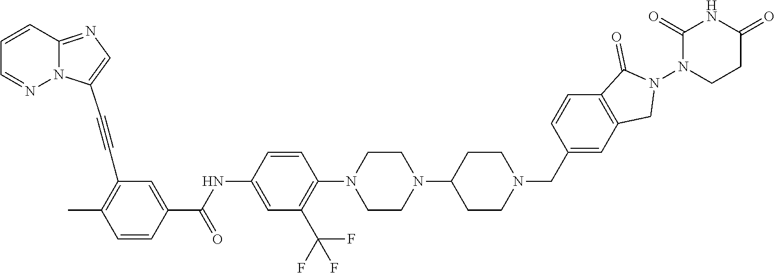 [SMILES: Cc1ccc(C(=O)Nc2ccc(N3CCN(C4CCN(Cc5ccc6c(c5)CN(N5CCC(=O)NC5=O)C6=O)CC4)CC3)c(C(F)(F)F)c2)cc1C#Cc1cnc2cccnn12] | N-(4-(4-(1-((2-(2,4- dioxotetrahydropyrimidin-1(2H)-yl)-1- oxoisoindolin-5-yl)methyl)piperidin-4- yl)piperazin-1-yl)-3-(trifluoromethyl)phenyl)- 3-(imidazo[1,2-b]pyridazin-3-ylethynyl)-4- methylbenzamide |
| GT- 0822 0 | 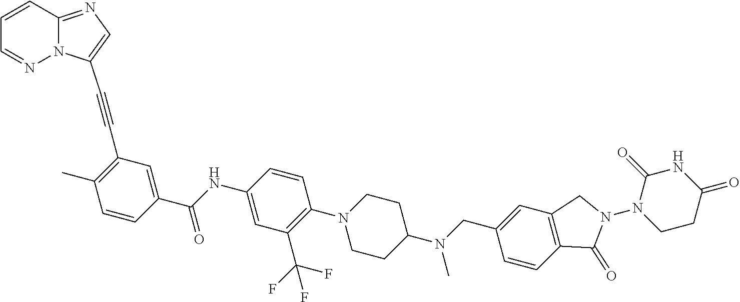 [SMILES: Cc1ccc(C(=O)Nc2ccc(N3CCC(N(C)Cc4ccc5c(c4)CN(N4CCC(=O)NC4=O)C5=O)CC3)c(C(F)(F)F)c2)cc1C#Cc1cnc2cccnn12] | N-(4-(4-(((2-(2,4-dioxotetrahydropyrimidin- 1(2H)-yl)-1-oxoisoindolin-5- yl)methyl)(methyl)amino)piperidin-1-yl)-3- (trifluoromethyl)phenyl)-3-(imidazo[1,2- b]pyridazin-3-ylethynyl)-4-methylbenzamide |
| GT- 0633 5 | 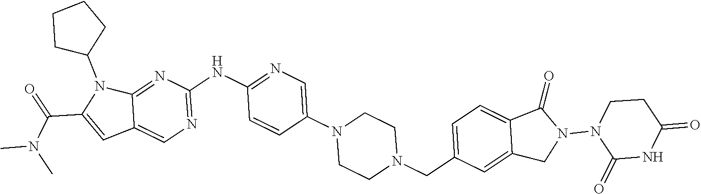 [SMILES: CN(C)C(=O)c1cc2cnc(Nc3ccc(N4CCN(Cc5ccc6c(c5)CN(N5CCC(=O)NC5=O)C6=O)CC4)cn3)nc2n1C1CCCC1] | 7-cyclopentyl-2-((5-(4-((2-(2,4- dioxotetrahydropyrimidin-1(2H)-yl)-1- oxoisoindolin-5-yl)methyl)piperazin-1- yl)pyridin-2-yl)amino)-N,N-dimethyl-7H- pyrrolo[2,3-d]pyrimidine-6-carboxamide |
| | 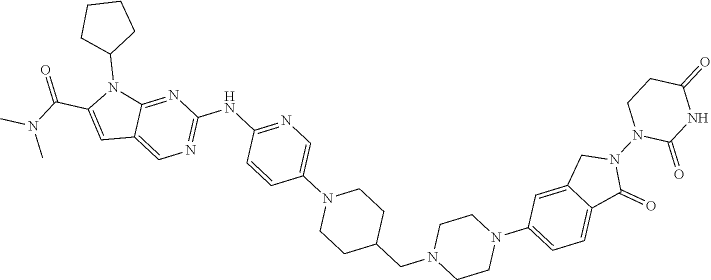 [SMILES: CN(C)C(=O)c1cc2cnc(Nc3ccc(N4CCC(CN5CCN(c6ccc7c(c6)CN(N6CCC(=O)NC6=O)C7=O)CC5)CC4)cn3)nc2n1C1CCCC1] | 7-cyclopentyl-2-((5-(4-((4-(2-(2,4- dioxotetrahydropyrimidin-1(2H)-yl)-1- oxoisoindolin-5-yl)piperazin-1- yl)methyl)piperidin-1-yl)pyridin-2-yl)amino)- N,N-dimethyl-7H-pyrrolo[2,3-d]pyrimidine- 6-carboxamide |
| GT- 0648 7 | 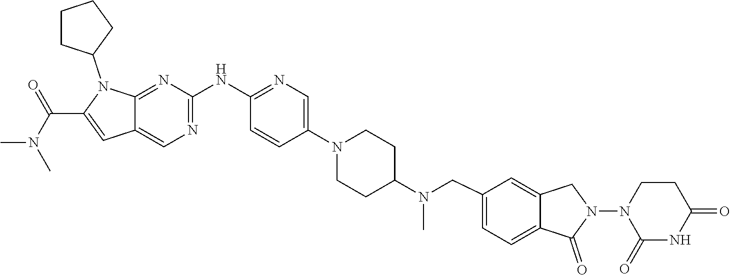 [SMILES: CN(C)C(=O)c1cc2cnc(Nc3ccc(N4CCC(N(C)Cc5ccc6c(c5)CN(N5CCC(=O)NC5=O)C6=O)CC4)cn3)nc2n1C1CCCC1] | 7-cyclopentyl-2-((5-(4-(((2-(2,4- dioxotetrahydropyrimidin-1(2H)-yl)-1- oxoisoindolin-5- yl)methyl)(methyl)amino)piperidin-1- yl)pyridin-2-yl)amino)-N,N-dimethyl-7H- pyrrolo[2,3-d]pyrimidine-6-carboxamide |
| GT- 0649 7 | 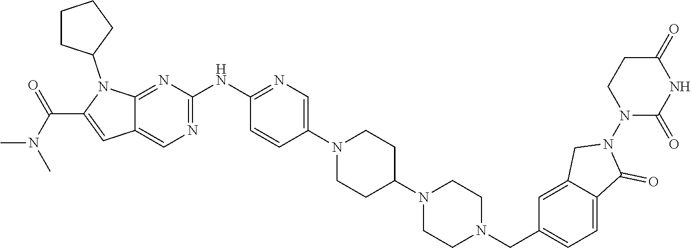 [SMILES: CN(C)C(=O)c1cc2cnc(Nc3ccc(N4CCC(N5CCN(Cc6ccc7c(c6)CN(N6CCC(=O)NC6=O)C7=O)CC5)CC4)cn3)nc2n1C1CCCC1] | 7-cyclopentyl-2-((5-(4-(4-((2-(2,4- dioxotetrahydropyrimidin-1(2H)-yl)-1- oxoisoindolin-5-yl)methyl)piperazin-1- yl)piperidin-1-yl)pyridin-2-yl)amino)-N,N- dimethyl-7H-pyrrolo[2,3-d]pyrimidine-6- carboxamide |
| | 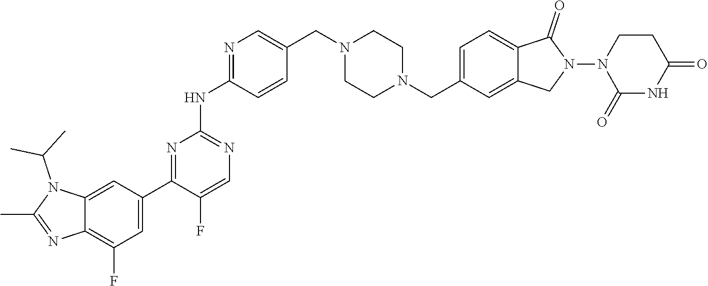 [SMILES: Cc1nc2c(F)cc(-c3nc(Nc4ccc(CN5CCN(Cc6ccc7c(c6)CN(N6CCC(=O)NC6=O)C7=O)CC5)cn4)ncc3F)cc2n1C(C)C] | 1-(5-((4-((6-((5-fluoro-4-(4-fluoro-1- isopropyl-2-methyl-1H-benzo[d]imidazol-6- yl)pyrimidin-2-yl)amino)pyridin-3- yl)methyl)piperazin-1-yl)methyl)-1- oxoisoindolin-2-yl)dihydropyrimidine- 2,4(1H,3H)-dione |
| GT- 0667 2 | 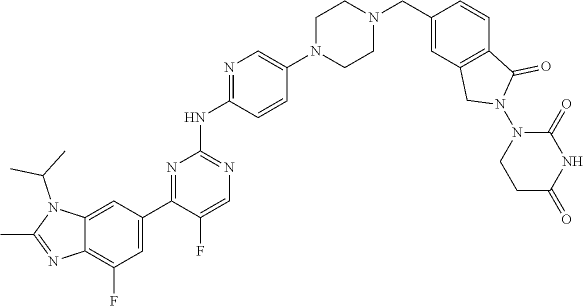 [SMILES: Cc1nc2c(F)cc(-c3nc(Nc4ccc(N5CCN(Cc6ccc7c(c6)CN(N6CCC(=O)NC6=O)C7=O)CC5)cn4)ncc3F)cc2n1C(C)C] | 1-(5-((4-(6-((5-fluoro-4-(4-fluoro-1- isopropyl-2-methyl-1H-benzo[d]imidazol-6- yl)pyrimidin-2-yl)amino)pyridin-3- yl)piperazin-1-yl)methyl)-1-oxoisoindolin-2- yl)dihydropyrimidine-2,4(1H,3H)-dione |
| GT- 0683 7 | 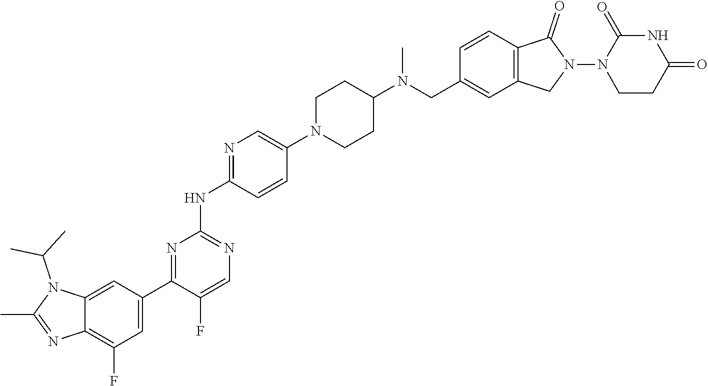 [SMILES: Cc1nc2c(F)cc(-c3nc(Nc4ccc(N5CCC(N(C)Cc6ccc7c(c6)CN(N6CCC(=O)NC6=O)C7=O)CC5)cn4)ncc3F)cc2n1C(C)C] | 1-(5-(((1-(6-((5-fluoro-4-(4-fluoro-1- isopropyl-2-methyl-1H-benzo[d]imidazol-6- yl)pyrimidin-2-yl)amino)pyridin-3- yl)piperidin-4-yl)(methyl)amino)methyl)-1- oxoisoindolin-2-yl)dihydropyrimidine- 2,4(1H,3H)-dione |
| GT- 0673 6 | 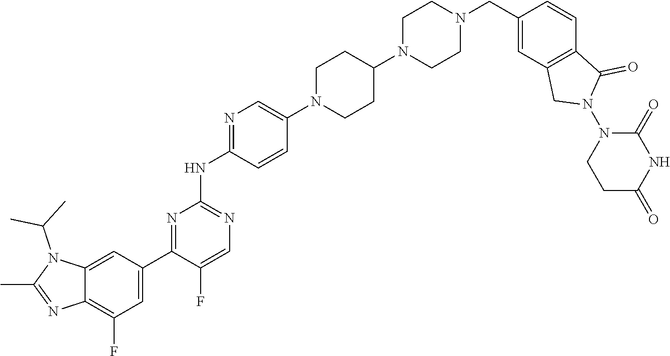 [SMILES: Cc1nc2c(F)cc(-c3nc(Nc4ccc(N5CCC(N6CCN(Cc7ccc8c(c7)CN(N7CCC(=O)NC7=O)C8=O)CC6)CC5)cn4)ncc3F)cc2n1C(C)C] | 1-(5-((4-(1-(6-((5-fluoro-4-(4-fluoro-1- isopropyl-2-methyl-1H-benzo[d]imidazol-6- yl)pyrimidin-2-yl)amino)pyridin-3- yl)piperidin-4-yl)piperazin-1-yl)methyl)-1- oxoisoindolin-2-yl)dihydropyrimidine- 2,4(1H,3H)-dione |
| GT- 0657 3 | 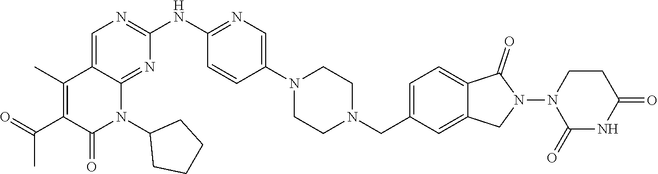 [SMILES: CC(=O)c1c(C)c2cnc(Nc3ccc(N4CCN(Cc5ccc6c(c5)CN(N5CCC(=O)NC5=O)C6=O)CC4)cn3)nc2n(C2CCCC2)c1=O] | 1-(5-((4-(6-((6-acetyl-8-cyclopentyl-5- methyl-7-oxo-7,8-dihydropyrido[2,3- d]pyrimidin-2-yl)amino)pyridin-3- yl)piperazin-1-yl)methyl)-1-oxoisoindolin-2- yl)dihydropyrimidine-2,4(1H,3H)-dione |
| GT- 0658 6 | 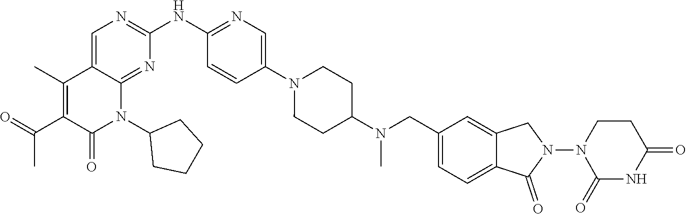 [SMILES: CC(=O)c1c(C)c2cnc(Nc3ccc(N4CCC(N(C)Cc5ccc6c(c5)CN(N5CCC(=O)NC5=O)C6=O)CC4)cn3)nc2n(C2CCCC2)c1=O] | 1-(5-(((1-(6-((6-acetyl-8-cyclopentyl-5- methyl-7-oxo-7,8-dihydropyrido[2,3- d]pyrimidin-2-yl)amino)pyridin-3- yl)piperidin-4-yl)(methyl)amino)methyl)-1- oxoisoindolin-2-yl)dihydropyrimidine- 2,4(1H,3H)-dione |
| | 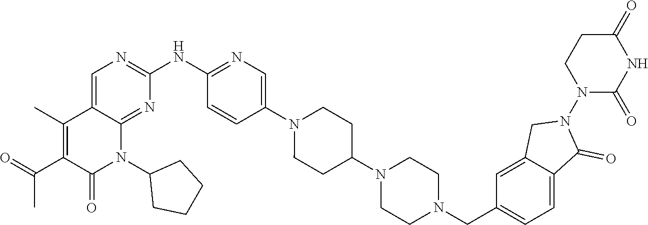 [SMILES: CC(=O)c1c(C)c2cnc(Nc3ccc(N4CCC(N5CCN(Cc6ccc7c(c6)CN(N6CCC(=O)NC6=O)C7=O)CC5)CC4)cn3)nc2n(C2CCCC2)c1=O] | 1-(5-((4-(1-(6-((6-acetyl-8-cyclopentyl-5- methyl-7-oxo-7,8-dihydropyrido[2,3- d]pyrimidin-2-yl)amino)pyridin-3- yl)piperidin-4-yl)piperazin-1-yl)methyl)-1- oxoisoindolin-2-yl)dihydropyrimidine- 2,4(1H,3H)-dione |
| GT- 0814 8 | 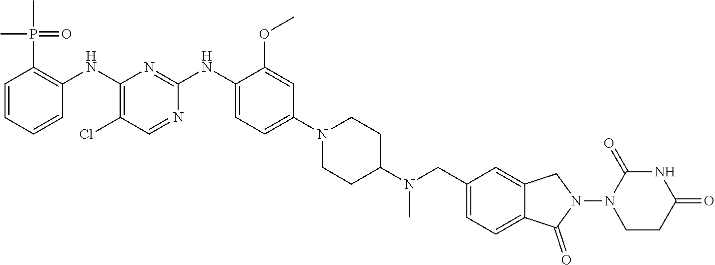 [SMILES: COc1cc(N2CCC(N(C)Cc3ccc4c(c3)CN(N3CCC(=O)NC3=O)C4=O)CC2)ccc1Nc1ncc(Cl)c(Nc2ccccc2P(C)(C)=O)n1] | 1-(5-(((1-(4-((5-chloro-4-((2- (dimethylphosphoryl)phenyl)amino)pyrimidin- 2-yl)amino)-3-methoxyphenyl)piperidin-4- yl)(methyl)amino)methyl)-1-oxoisoindolin-2- yl)dihydropyrimidine-2,4(1H,3H)-dione |
| | 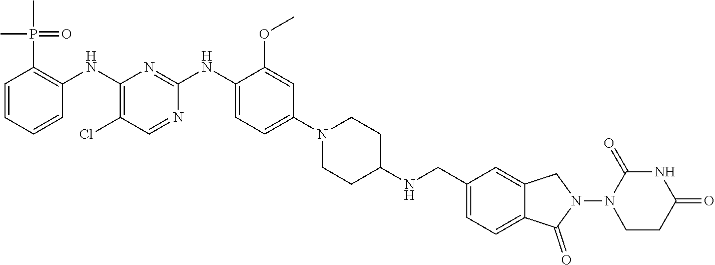 [SMILES: COc1cc(N2CCC(NCc3ccc4c(c3)CN(N3CCC(=O)NC3=O)C4=O)CC2)ccc1Nc1ncc(Cl)c(Nc2ccccc2P(C)(C)=O)n1] | 1-(5-(((1-(4-((5-chloro-4-((2- (dimethylphosphoryl)phenyl)amino)pyrimidin- 2-yl)amino)-3-methoxyphenyl)piperidin-4- yl)amino)methyl)-1-oxoisoindolin-2- yl)dihydropyrimidine-2,4(1H,3H)-dione |
| GT- 0734 1 | 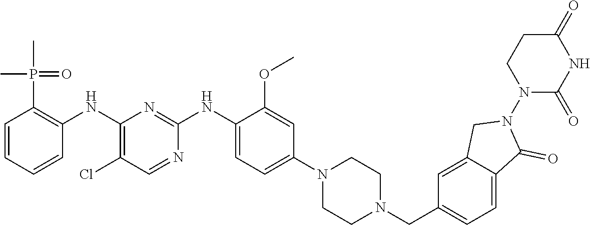 [SMILES: COc1cc(N2CCN(Cc3ccc4c(c3)CN(N3CCC(=O)NC3=O)C4=O)CC2)ccc1Nc1ncc(Cl)c(Nc2ccccc2P(C)(C)=O)n1] | 1-(5-((4-(4-((5-chloro-4-((2- (dimethylphosphoryl)phenyl)amino)pyrimidin- 2-yl)amino)-3-methoxyphenyl)piperazin-1- yl)methyl)-1-oxoisoindolin-2- yl)dihydropyrimidine-2,4(1H,3H)-dione |
| GT- 0814 6 | 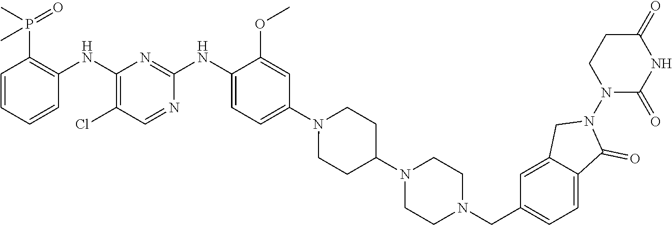 [SMILES: COc1cc(N2CCC(N3CCN(Cc4ccc5c(c4)CN(N4CCC(=O)NC4=O)C5=O)CC3)CC2)ccc1Nc1ncc(Cl)c(Nc2ccccc2P(C)(C)=O)n1] | 1-(5-((4-(1-(4-((5-chloro-4-((2- (dimethylphosphoryl)phenyl)amino)pyrimidin- 2-yl)amino)-3-methoxyphenyl)piperidin-4- yl)piperazin-1-yl)methyl)-1-oxoisoindolin-2- yl)dihydropyrimidine-2,4(1H,3H)-dione |
| GT- 0682 2 | 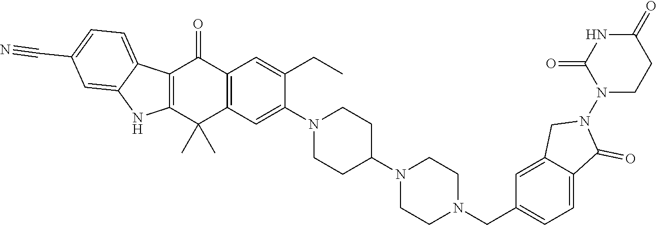 [SMILES: CCc1cc2c(cc1N1CCC(N3CCN(Cc4ccc5c(c4)CN(N4CCC(=O)NC4=O)C5=O)CC3)CC1)C(C)(C)c1[nH]c3cc(C#N)ccc3c1C2=O] | 8-(4-(4-((2-(2,4-dioxotetrahydropyrimidin- 1(2H)-yl)-1-oxoisoindolin-5- yl)methyl)piperazin-1-yl)piperidin-1-yl)-9- ethyl-6,6-dimethyl-11-oxo-6,11-dihydro-5H- benzo[b]carbazole-3-carbonitrile |
| GT- 0734 0 | 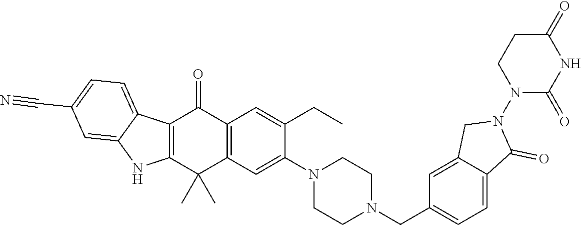 [SMILES: CCc1cc2c(cc1N1CCN(Cc3ccc4c(c3)CN(N3CCC(=O)NC3=O)C4=O)CC1)C(C)(C)c1[nH]c3cc(C#N)ccc3c1C2=O] | 8-(4-((2-(2,4-dioxotetrahydropyrimidin- 1(2H)-yl)-1-oxoisoindolin-5- yl)methyl)piperazin-1-yl)-9-ethyl-6,6- dimethyl-11-oxo-6,11-dihydro-5H- benzo[b]carbazole-3-carbonitrile |
| | 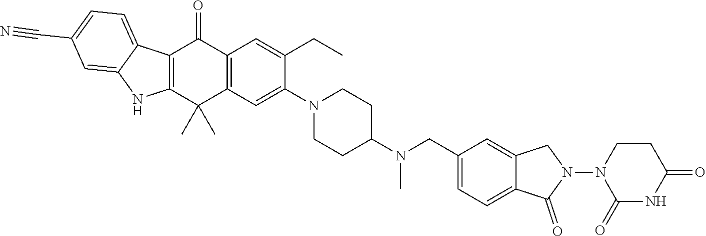 [SMILES: CCc1cc2c(cc1N1CCC(N(C)Cc3ccc4c(c3)CN(N3CCC(=O)NC3=O)C4=O)CC1)C(C)(C)c1[nH]c3cc(C#N)ccc3c1C2=O] | 8-(4-(((2-(2,4-dioxotetrahydropyrimidin- 1(2H)-yl)-1-oxoisoindolin-5- yl)methyl)(methyl)amino)piperidin-1-yl)-9- ethyl-6,6-dimethyl-11-oxo-6,11-dihydro-5H- benzo[b]carbazole-3-carbonitrile |
| | 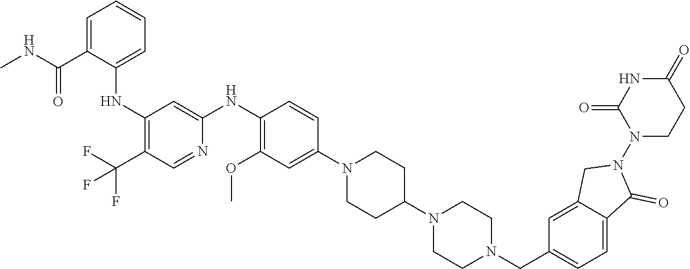 [SMILES: CNC(=O)c1ccccc1Nc1cc(Nc2ccc(N3CCC(N4CCN(Cc5ccc6c(c5)CN(N5CCC(=O)NC5=O)C6=O)CC4)CC3)cc2OC)ncc1C(F)(F)F] | 2-((2-((4-(4-(4-((2-(2,4- dioxotetrahydropyrimidin-1(2H)-yl)-1- oxoisoindolin-5-yl)methyl)piperazin-1- yl)piperidin-1-yl)-2-methoxyphenyl)amino)- 5-(trifluoromethyl)pyridin-4-yl)amino)-N- methylbenzamide |
| | 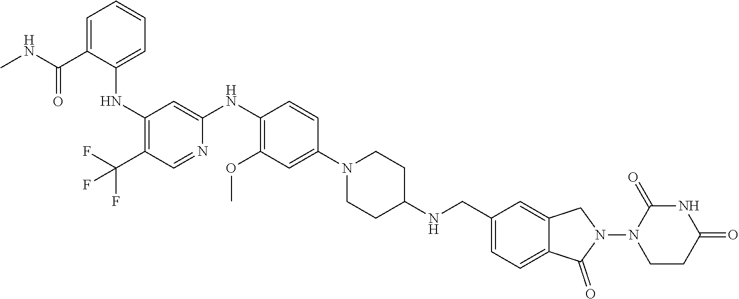 [SMILES: CNC(=O)c1ccccc1Nc1cc(Nc2ccc(N3CCC(NCc4ccc5c(c4)CN(N4CCC(=O)NC4=O)C5=O)CC3)cc2OC)ncc1C(F)(F)F] | 2-((2-((4-(4-(((2-(2,4- dioxotetrahydropyrimidin-1(2H)-yl)-1- oxoisoindolin-5-yl)methyl)amino)piperidin-1- yl)-2-methoxyphenyl)amino)-5- (trifluoromethyl)pyridin-4-yl)amino)-N- methylbenzamide |
| GT- 0764 7 | 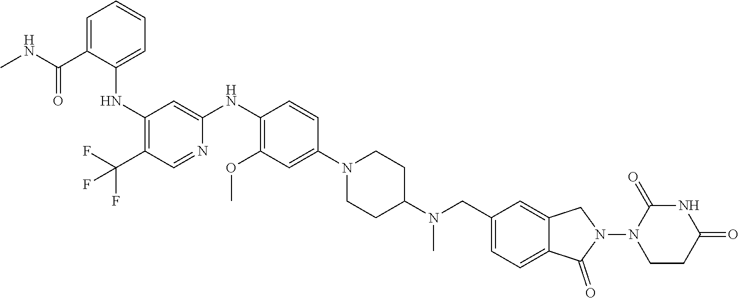 [SMILES: CNC(=O)c1ccccc1Nc1cc(Nc2ccc(N3CCC(N(C)Cc4ccc5c(c4)CN(N4CCC(=O)NC4=O)C5=O)CC3)cc2OC)ncc1C(F)(F)F] | 2-((2-((4-(4-(((2-(2,4- dioxotetrahydropyrimidin-1(2H)-yl)-1- oxoisoindolin-5- yl)methyl)(methyl)amino)piperidin-1-yl)-2- methoxyphenyl)amino)-5- (trifluoromethyl)pyridin-4-yl)amino)-N- methylbenzamide |
| GT- 0766 0 | 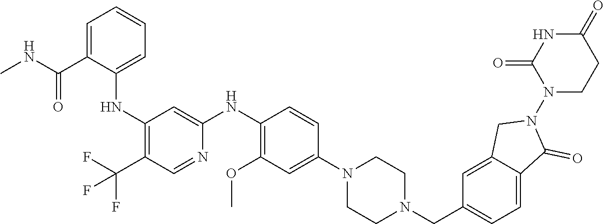 [SMILES: CNC(=O)c1ccccc1Nc1cc(Nc2ccc(N3CCN(Cc4ccc5c(c4)CN(N4CCC(=O)NC4=O)C5=O)CC3)cc2OC)ncc1C(F)(F)F] | 2-((2-((4-(4-((2-(2,4- dioxotetrahydropyrimidin-1(2H)-yl)-1- oxoisoindolin-5-yl)methyl)piperazin-1-yl)-2- methoxyphenyl)amino)-5- (trifluoromethyl)pyridin-4-yl)amino)-N- methylbenzamide |
| GT- 0766 3 | 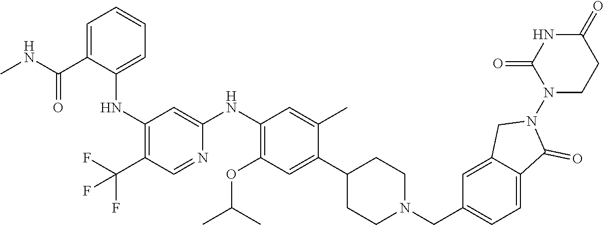 [SMILES: CNC(=O)c1ccccc1Nc1cc(Nc2cc(C)c(C3CCN(Cc4ccc5c(c4)CN(N4CCC(=O)NC4=O)C5=O)CC3)cc2OC(C)C)ncc1C(F)(F)F] | 2-((2-((4-(1-((2-(2,4- dioxotetrahydropyrimidin-1(2H)-yl)-1- oxoisoindolin-5-yl)methyl)piperidin-4-yl)-2- isopropoxy-5-methylphenyl)amino)-5- (trifluoromethyl)pyridin-4-yl)amino)-N- methylbenzamide |
| | 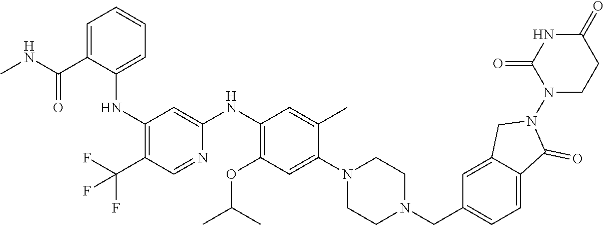 [SMILES: CNC(=O)c1ccccc1Nc1cc(Nc2cc(C)c(N3CCN(Cc4ccc5c(c4)CN(N4CCC(=O)NC4=O)C5=O)CC3)cc2OC(C)C)ncc1C(F)(F)F] | 2-((2-((4-(4-((2-(2,4- dioxotetrahydropyrimidin-1(2H)-yl)-1- oxoisoindolin-5-yl)methyl)piperazin-1-yl)-2- isopropoxy-5-methylphenyl)amino)-5- (trifluoromethyl)pyridin-4-yl)amino)-N- methylbenzamide |
| | 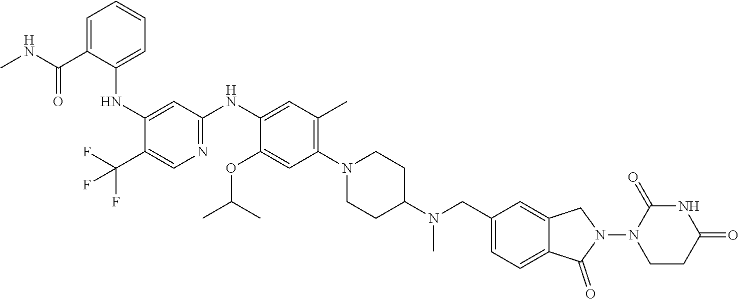 [SMILES: CNC(=O)c1ccccc1Nc1cc(Nc2cc(C)c(N3CCC(N(C)Cc4ccc5c(c4)CN(N4CCC(=O)NC4=O)C5=O)CC3)cc2OC(C)C)ncc1C(F)(F)F] | 2-((2-((4-(4-(((2-(2,4- dioxotetrahydropyrimidin-1(2H)-yl)-1- oxoisoindolin-5- yl)methyl)(methyl)amino)piperidin-1-yl)-2- isopropoxy-5-methylphenyl)amino)-5- (trifluoromethyl)pyridin-4-yl)amino)-N- methylbenzamide |
| | 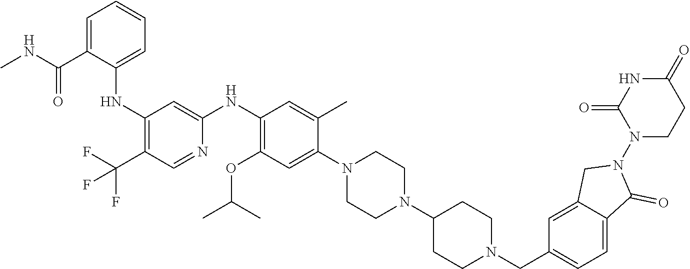 [SMILES: CNC(=O)c1ccccc1Nc1cc(Nc2cc(C)c(N3CCN(C4CCN(Cc5ccc6c(c5)CN(N5CCC(=O)NC5=O)C6=O)CC4)CC3)cc2OC(C)C)ncc1C(F)(F)F] | 2-((2-((4-(4-(1-((2-(2,4- dioxotetrahydropyrimidin-1(2H)-yl)-1- oxoisoindolin-5-yl)methyl)piperidin-4- yl)piperazin-1-yl)-2-isopropoxy-5- methylphenyl)amino)-5- (trifluoromethyl)pyridin-4-yl)amino)-N- methylbenzamide |
| | 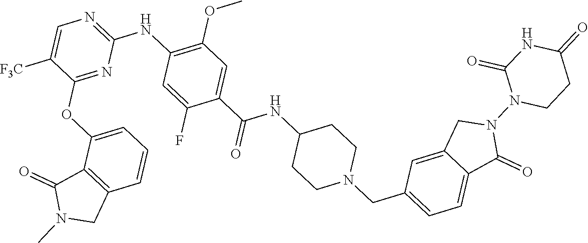 [SMILES: COc1cc(C(=O)NC2CCN(Cc3ccc4c(c3)CN(N3CCC(=O)NC3=O)C4=O)CC2)c(F)cc1Nc1ncc(C(F)(F)F)c(Oc2cccc3c2C(=O)N(C)C3)n1] | N-(1-((2-(2,4-dioxotetrahydropyrimidin- 1(2H)-yl)-1-oxoisoindolin-5- yl)methyl)piperidin-4-yl)-2-fluoro-5- methoxy-4-((4-((2-methyl-3-oxoisoindolin-4- yl)oxy)-5-(trifluoromethyl)pyrimidin-2- yl)amino)benzamide |
| GT- 0750 4 | 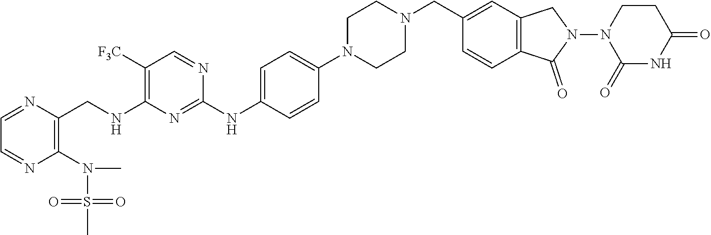 [SMILES: CN(c1nccnc1CNc1nc(Nc2ccc(N3CCN(Cc4ccc5c(c4)CN(N4CCC(=O)NC4=O)C5=O)CC3)cc2)ncc1C(F)(F)F)S(C)(=O)=O] | N-(3-(((2-((4-(4-((2-(2,4- dioxotetrahydropyrimidin-1(2H)-yl)-1- oxoisoindolin-5-yl)methyl)piperazin-1- yl)phenyl)amino)-5- (trifluoromethyl)pyrimidin-4- yl)amino)methyl)pyrazin-2-yl)-N- methylmethanesulfonamide |
| GT- 0813 6 | 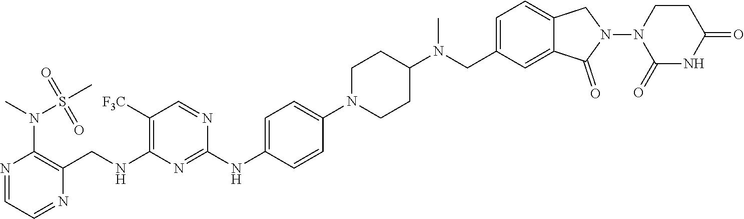 [SMILES: CN(Cc1ccc2c(c1)C(=O)N(N1CCC(=O)NC1=O)C2)C1CCN(c2ccc(Nc3ncc(C(F)(F)F)c(NCc4nccnc4N(C)S(C)(=O)=O)n3)cc2)CC1] | N-(3-(((2-((4-(4-(((2-(2,4- dioxotetrahydropyrimidin-1(2H)-yl)-3- oxoisoindolin-5- yl)methyl)(methyl)amino)piperidin-1- yl)phenyl)amino)-5- (trifluoromethyl)pyrimidin-4- yl)amino)methyl)pyrazin-2-yl)-N- methylmethanesulfonamide |
| GT- 0813 8 | 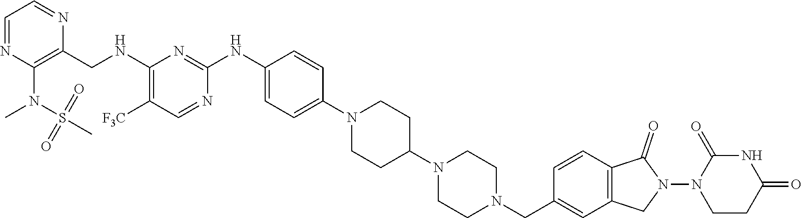 [SMILES: CN(c1nccnc1CNc1nc(Nc2ccc(N3CCC(N4CCN(Cc5ccc6c(c5)CN(N5CCC(=O)NC5=O)C6=O)CC4)CC3)cc2)ncc1C(F)(F)F)S(C)(=O)=O] | N-(3-(((2-((4-(4-(4-((2-(2,4- dioxotetrahydropyrimidin-1(2H)-yl)-1- oxoisoindolin-5-yl)methyl)piperazin-1- yl)piperidin-1-yl)phenyl)amino)-5- (trifluoromethyl)pyrimidin-4- yl)amino)methyl)pyrazin-2-yl)-N- methylmethanesulfonamide |
| GT- 0814 0 | 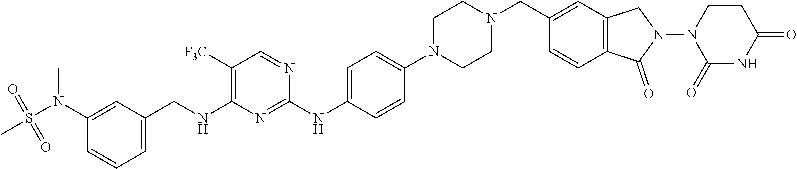 [SMILES: CN(c1cccc(CNc2nc(Nc3ccc(N4CCN(Cc5ccc6c(c5)CN(N5CCC(=O)NC5=O)C6=O)CC4)cc3)ncc2C(F)(F)F)c1)S(C)(=O)=O] | N-(3-(((2-((4-(4-((2-(2,4- dioxotetrahydropyrimidin-1(2H)-yl)-1- oxoisoindolin-5-yl)methyl)piperazin-1- yl)phenyl)amino)-5- (trifluoromethyl)pyrimidin-4- yl)amino)methyl)phenyl)-N- methylmethanesulfonamide |
| | 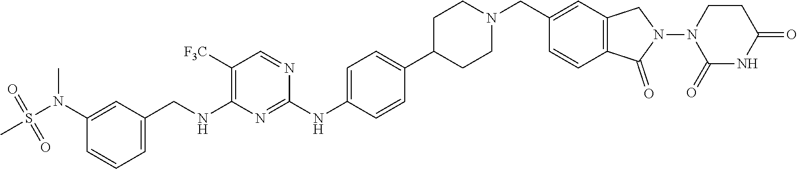 [SMILES: CN(c1cccc(CNc2nc(Nc3ccc(C4CCN(Cc5ccc6c(c5)CN(N5CCC(=O)NC5=O)C6=O)CC4)cc3)ncc2C(F)(F)F)c1)S(C)(=O)=O] | N-(3-(((2-((4-(1-((2-(2,4- dioxotetrahydropyrimidin-1(2H)-yl)-1- oxoisoindolin-5-yl)methyl)piperidin-4- yl)phenyl)amino)-5- (trifluoromethyl)pyrimidin-4- yl)amino)methyl)phenyl)-N- methylmethanesulfonamide |
| GT- 0814 2 | 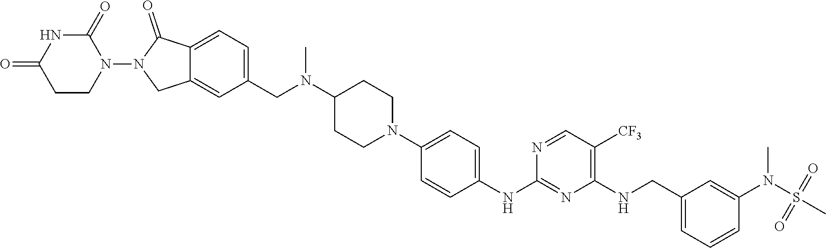 [SMILES: CN(Cc1ccc2c(c1)CN(N1CCC(=O)NC1=O)C2=O)C1CCN(c2ccc(Nc3ncc(C(F)(F)F)c(NCc4cccc(N(C)S(C)(=O)=O)c4)n3)cc2)CC1] | N-(3-(((2-((4-(4-(((2-(2,4- dioxotetrahydropyrimidin-1(2H)-yl)-1- oxoisoindolin-5- yl)methyl)(methyl)amino)piperidin-1- yl)phenyl)amino)-5- (trifluoromethyl)pyrimidin-4- yl)amino)methyl)phenyl)-N- methylmethanesulfonamide |
| GT- 0814 4 | 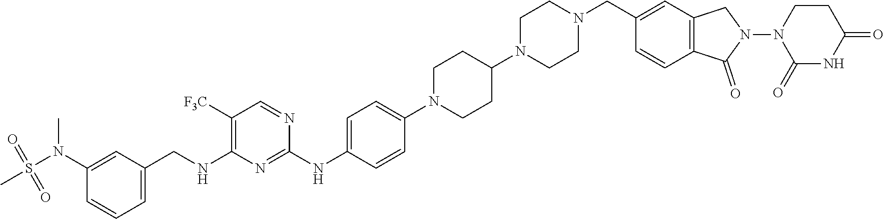 [SMILES: CN(c1cccc(CNc2nc(Nc3ccc(N4CCC(N5CCN(Cc6ccc7c(c6)CN(N6CCC(=O)NC6=O)C7=O)CC5)CC4)cc3)ncc2C(F)(F)F)c1)S(C)(=O)=O] | N-(3-(((2-((4-(4-(4-((2-(2,4- dioxotetrahydropyrimidin-1(2H)-yl)-1- oxoisoindolin-5-yl)methyl)piperazin-1- yl)piperidin-1-yl)phenyl)amino)-5- (trifluoromethyl)pyrimidin-4- yl)amino)methyl)phenyl)-N- methylmethanesulfonamide |
| GT- 0751 4 | 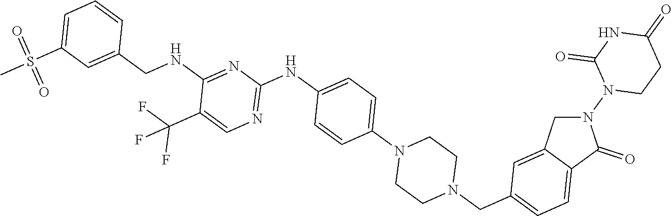 [SMILES: CS(=O)(=O)c1cccc(CNc2nc(Nc3ccc(N4CCN(Cc5ccc6c(c5)CN(N5CCC(=O)NC5=O)C6=O)CC4)cc3)ncc2C(F)(F)F)c1] | 1-(5-((4-(4-((4-((3- (methylsulfonyl)benzyl)amino)-5- (trifluoromethyl)pyrimidin-2- yl)amino)phenyl)piperazin-1-yl)methyl)-1- oxoisoindolin-2-yl)dihydropyrimidine- 2,4(1H,3H)-dione |
| GT- 0495 1 | 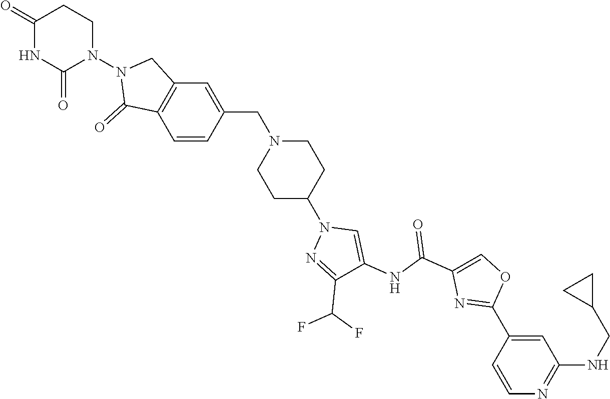 [SMILES: O=C1CCN(N2Cc3cc(CN4CCC(n5cc(NC(=O)c6coc(-c7ccnc(NCC8CC8)c7)n6)c(C(F)F)n5)CC4)ccc3C2=O)C(=O)N1] | 2-(2-((cyclopropylmethyl)amino)pyridin-4- yl)-N-(3-(difluoromethyl)-1-(1-((2-(2,4- dioxotetrahydropyrimidin-1(2H)-yl)-1- oxoisoindolin-5-yl)methyl)piperidin-4-yl)- 1H-pyrazol-4-yl)oxazole-4-carboxamide |
| | 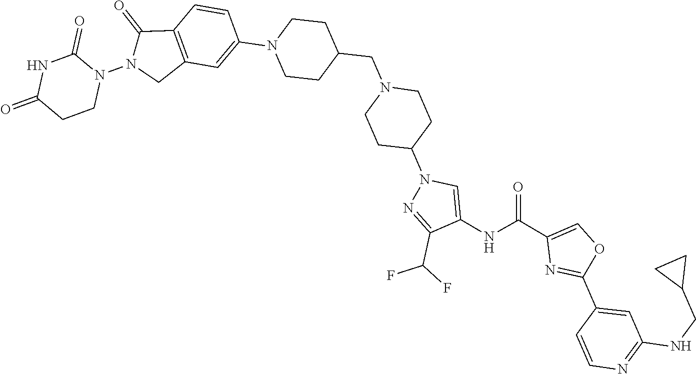 [SMILES: O=C1CCN(N2Cc3cc(N4CCC(CN5CCC(n6cc(NC(=O)c7coc(-c8ccnc(NCC9CC9)c8)n7)c(C(F)F)n6)CC5)CC4)ccc3C2=O)C(=O)N1] | 2-(2-((cyclopropylmethyl)amino)pyridin-4- yl)-N-(3-(difluoromethyl)-1-(1-((1-(2-(2,4- dioxotetrahydropyrimidin-1(2H)-yl)-1- oxoisoindolin-5-yl)piperidin-4- yl)methyl)piperidin-4-yl)-1H-pyrazol-4- yl)oxazole-4-carboxamide |
| | 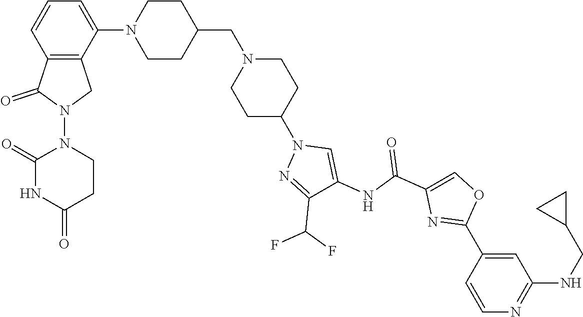 [SMILES: O=C1CCN(N2Cc3c(cccc3N3CCC(CN4CCC(n5cc(NC(=O)c6coc(-c7ccnc(NCC8CC8)c7)n6)c(C(F)F)n5)CC4)CC3)C2=O)C(=O)N1] | 2-(2-((cyclopropylmethyl)amino)pyridin-4- yl)-N-(3-(difluoromethyl)-1-(1-((1-(2-(2,4- dioxotetrahydropyrimidin-1(2H)-yl)-1- oxoisoindolin-4-yl)piperidin-4- yl)methyl)piperidin-4-yl)-1H-pyrazol-4- yl)oxazole-4-carboxamide |
| | 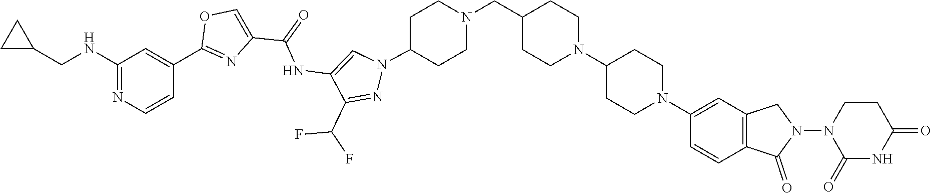 [SMILES: O=C1CCN(N2Cc3cc(N4CCC(N5CCC(CN6CCC(n7cc(NC(=O)c8coc(-c9ccnc(NCC%10CC%10)c9)n8)c(C(F)F)n7)CC6)CC5)CC4)ccc3C2=O)C(=O)N1] | 2-(2-((cyclopropylmethyl)amino)pyridin-4- yl)-N-(3-(difluoromethyl)-1-(1-((1′-(2-(2,4- dioxotetrahydropyrimidin-1(2H)-yl)-1- oxoisoindolin-5-yl)-[1,4′-bipiperidin]-4- yl)methyl)piperidin-4-yl)-1H-pyrazol-4- yl)oxazole-4-carboxamide |
| | 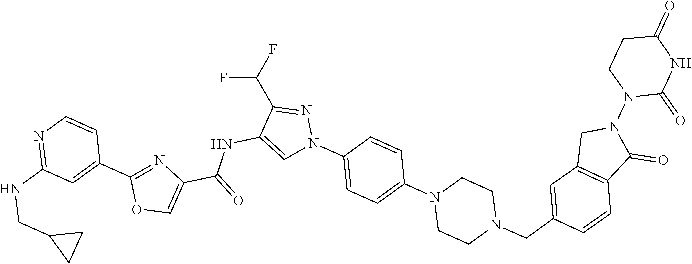 [SMILES: O=C1CCN(N2Cc3cc(CN4CCN(c5ccc(-n6cc(NC(=O)c7coc(-c8ccnc(NCC9CC9)c8)n7)c(C(F)F)n6)cc5)CC4)ccc3C2=O)C(=O)N1] | 2-(2-((cyclopropylmethyl)amino)pyridin-4- yl)-N-(3-(difluoromethyl)-1-(4-(4-((2-(2,4- dioxotetrahydropyrimidin-1(2H)-yl)-1- oxoisoindolin-5-yl)methyl)piperazin-1- yl)phenyl)-1H-pyrazol-4-yl)oxazole-4- carboxamide |
| GT- 0487 6 | 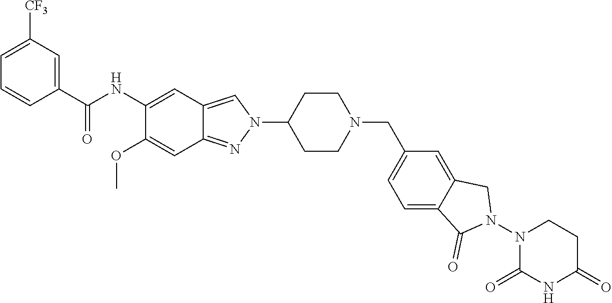 [SMILES: COc1cc2nn(C3CCN(Cc4ccc5c(c4)CN(N4CCC(=O)NC4=O)C5=O)CC3)cc2cc1NC(=O)c1cccc(C(F)(F)F)c1] | N-(2-(1-((2-(2,4-dioxotetrahydropyrimidin- 1(2H)-yl)-1-oxoisoindolin-5- yl)methyl)piperidin-4-yl)-6-methoxy-2H- indazol-5-yl)-3-(trifluoromethyl)benzamide |
| | 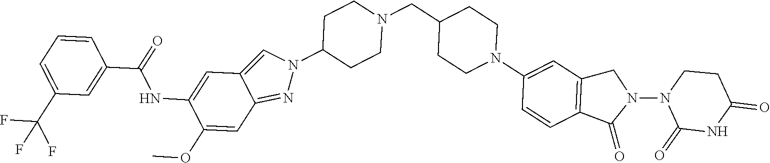 [SMILES: COc1cc2nn(C3CCN(CC4CCN(c5ccc6c(c5)CN(N5CCC(=O)NC5=O)C6=O)CC4)CC3)cc2cc1NC(=O)c1cccc(C(F)(F)F)c1] | N-(2-(1-((1-(2-(2,4- dioxotetrahydropyrimidin-1(2H)-yl)-1- oxoisoindolin-5-yl)piperidin-4- yl)methyl)piperidin-4-yl)-6-methoxy-2H- indazol-5-yl)-3-(trifluoromethyl)benzamide |
| GT- 0488 0 | 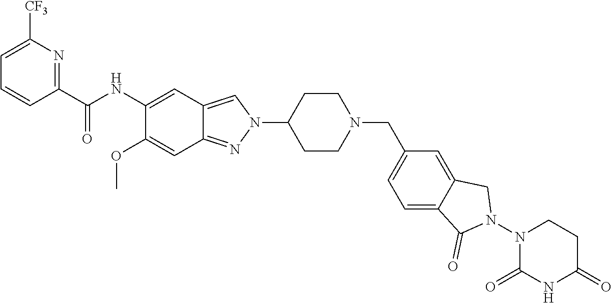 [SMILES: COc1cc2nn(C3CCN(Cc4ccc5c(c4)CN(N4CCC(=O)NC4=O)C5=O)CC3)cc2cc1NC(=O)c1cccc(C(F)(F)F)n1] | N-(2-(1-((2-(2,4-dioxotetrahydropyrimidin- 1(2H)-yl)-1-oxoisoindolin-5- yl)methyl)piperidin-4-yl)-6-methoxy-2H- indazol-5-yl)-6-(trifluoromethyl)picolinamide |
| | 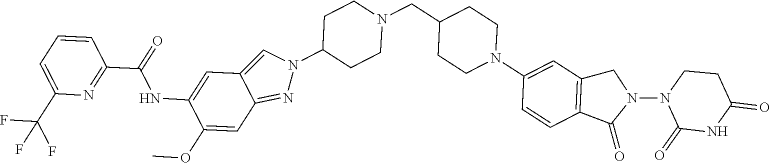 [SMILES: COc1cc2nn(C3CCN(CC4CCN(c5ccc6c(c5)CN(N5CCC(=O)NC5=O)C6=O)CC4)CC3)cc2cc1NC(=O)c1cccc(C(F)(F)F)n1] | N-(2-(1-((1-(2-(2,4- dioxotetrahydropyrimidin-1(2H)-yl)-1- oxoisoindolin-5-yl)piperidin-4- yl)methyl)piperidin-4-yl)-6-methoxy-2H- indazol-5-yl)-6-(trifluoromethyl)picolinamide |
| GT- 0488 4 | 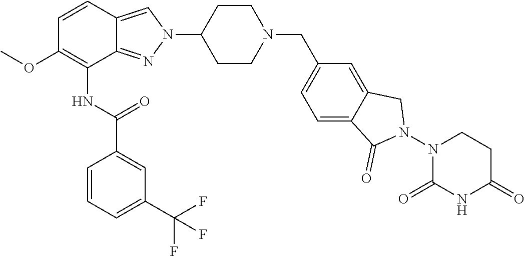 [SMILES: COc1ccc2cn(C3CCN(Cc4ccc5c(c4)CN(N4CCC(=O)NC4=O)C5=O)CC3)nc2c1NC(=O)c1cccc(C(F)(F)F)c1] | N-(2-(1 -((2-(2,4-dioxotetrahydropyrimidin- 1(2H)-yl)-1-oxoisoindolin-5- yl)methyl)piperidin-4-yl)-6-methoxy-2H- indazol-7-yl)-3-(trifluoromethyl)benzamide |
| GT- 0574 9 | 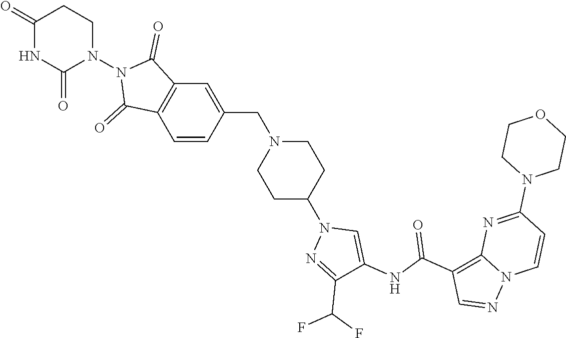 [SMILES: O=C1CCN(N2C(=O)c3ccc(CN4CCC(n5cc(NC(=O)c6cnn7ccc(N8CCOCC8)nc67)c(C(F)F)n5)CC4)cc3C2=O)C(=O)N1] | N-(3-(difluoromethyl)-1-(1-((2-(2,4- dioxotetrahydropyrimidin-1(2H)-yl)-1- oxoisoindolin-5-yl)methyl)piperidin-4-yl)- 1H-pyrazol-4-yl)-5-morpholinopyrazolo[1,5- a]pyrimidine-3-carboxamide |
| GT- 0502 8 | 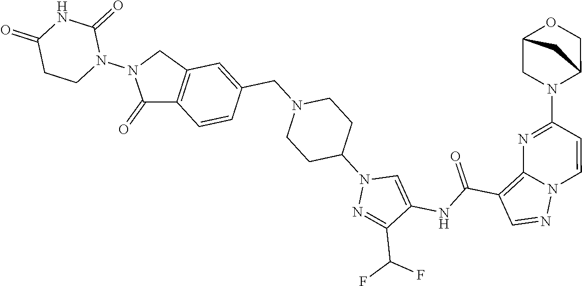 [SMILES: O=C1CCN(N2Cc3cc(CN4CCC(n5cc(NC(=O)c6cnn7ccc(N8C[C@H]9C[C@@H]8CO9)nc67)c(C(F)F)n5)CC4)ccc3C2=O)C(=O)N1] | 5-((1R,4R)-2-oxa-5-azabicyclo[2.2.1]heptan- 5-yl)-N-(3-(difluoromethyl)-1-(1-((2-(2,4- dioxotetrahydropyrimidin-1(2H)-yl)-1- oxoisoindolin-5-yl)methyl)piperidin-4-yl)- 1H-pyrazol-4-yl)pyrazolo[1,5-a]pyrimidine- 3-carboxamide |
| | 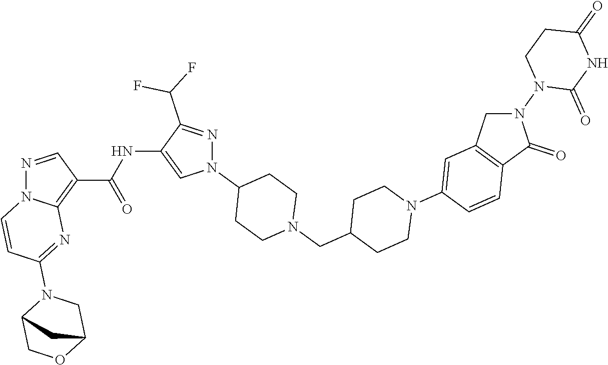 [SMILES: O=C1CCN(N2Cc3cc(N4CCC(CN5CCC(n6cc(NC(=O)c7cnn8ccc(N9C[C@H]%10C[C@@H]9CO%10)nc78)c(C(F)F)n6)CC5)CC4)ccc3C2=O)C(=O)N1] | 5-((1R,4R)-2-oxa-5-azabicyclo[2.2.1]heptan- 5-yl)-N-(3-(difluoromethyl)-1-(1-((1-(2-(2,4- dioxotetrahydropyrimidin-1(2H)-yl)-1- oxoisoindolin-5-yl)piperidin-4- yl)methyl)piperidin-4-yl)-1H-pyrazol-4- yl)pyrazolo[1,5-a]pyrimidine-3-carboxamide |
| | 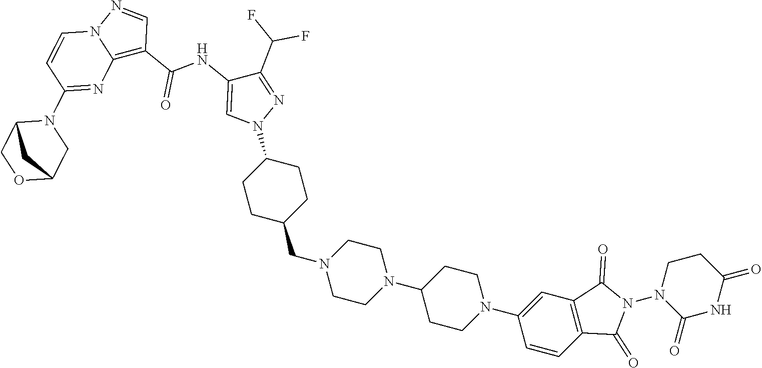 [SMILES: O=C1CCN(N2C(=O)c3ccc(N4CCC(N5CCN(C[C@H]6CC[C@H](n7cc(NC(=O)c8cnn9ccc(N%10C[C@H]%11C[C@@H]%10CO%11)nc89)c(C(F)F)n7)CC6)CC5)CC4)cc3C2=O)C(=O)N1] | 5-((1R,4R)-2-oxa-5-azabicyclo[2.2.1]heptan- 5-yl)-N-(3-(difluoromethyl)-1-((1r,4R)-4-((4- (1-(2-(2,4-dioxotetrahydropyrimidin-1(2H)- yl)-1-oxoisoindolin-5-yl)piperidin-4- yl)piperazin-1-yl)methyl)cyclohexyl)-1H- pyrazol-4-yl)pyrazolo[1,5-a]pyrimidine-3- carboxamide |
| | 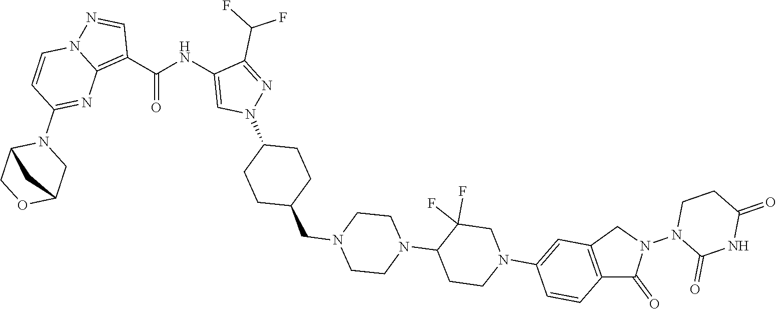 [SMILES: O=C1CCN(N2Cc3cc(N4CCC(N5CCN(C[C@H]6CC[C@H](n7cc(NC(=O)c8cnn9ccc(N%10C[C@H]%11C[C@@H]%10CO%11)nc89)c(C(F)F)n7)CC6)CC5)C(F)(F)C4)ccc3C2=O)C(=O)N1] | 5-((1R,4R)-2-oxa-5-azabicyclo[2.2.1]heptan- 5-yl)-N-(3-(difluoromethyl)-1-((1r,4R)-4-((4- (1-(2-(2,4-dioxotetrahydropyrimidin-1(2H)- yl)-1-oxoisoindolin-5-yl)-3,3- difluoropiperidin-4-yl)piperazin-1- yl)methyl)cyclohexyl)-1H-pyrazol-4- yl)pyrazolo[1,5-a]pyrimidine-3-carboxamide |
| | 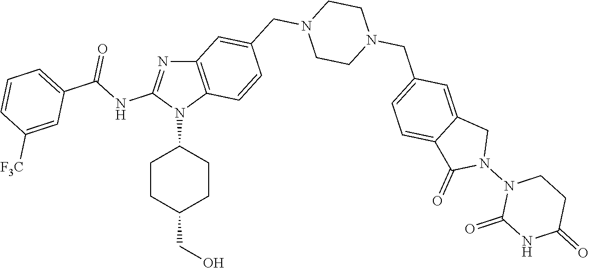 [SMILES: O=C1CCN(N2Cc3cc(CN4CCN(Cc5ccc6c(c5)nc(NC(=O)c5cccc(C(F)(F)F)c5)n6[C@H]5CC[C@@H](CO)CC5)CC4)ccc3C2=O)C(=O)N1] | N-(5-((4-((2-(2,4-dioxotetrahydropyrimidin- 1(2H)-yl)-1-oxoisoindolin-5- yl)methyl)piperazin-1-yl)methyl)-1-((1s,4s)- 4-(hydroxymethyl)cyclohexyl)-1H- benzo[d]imidazol-2-yl)-3- (trifluoromethyl)benzamide |
| GT- 0494 9 | 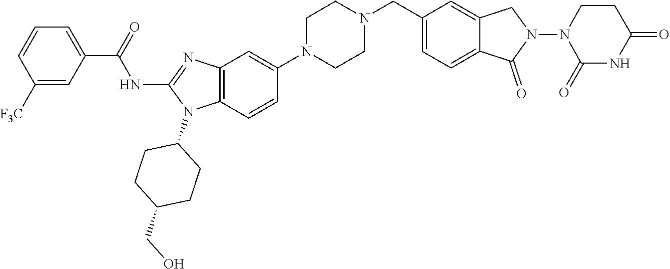 [SMILES: O=C1CCN(N2Cc3cc(CN4CCN(c5ccc6c(c5)nc(NC(=O)c5cccc(C(F)(F)F)c5)n6[C@H]5CC[C@@H](CO)CC5)CC4)ccc3C2=O)C(=O)N1] | N-(5-(4-((2-(2,4-dioxotetrahydropyrimidin- 1(2H)-yl)-1-oxoisoindolin-5- yl)methyl)piperazin-1-yl)-1-((1s,4s)-4- (hydroxymethyl)cyclohexyl)-1H- benzo[d]imidazol-2-yl)-3- (trifluoromethyl)benzamide |
| | 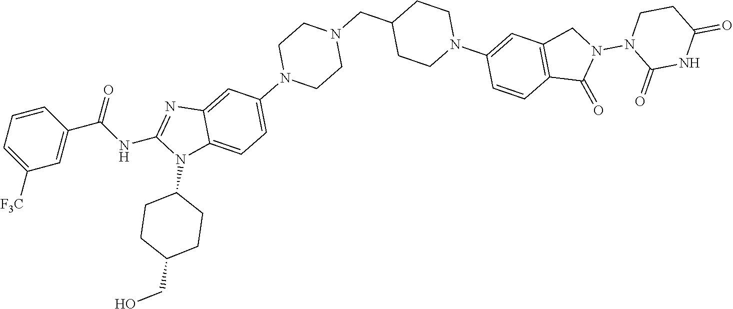 [SMILES: O=C1CCN(N2Cc3cc(N4CCC(CN5CCN(c6ccc7c(c6)nc(NC(=O)c6cccc(C(F)(F)F)c6)n7[C@H]6CC[C@@H](CO)CC6)CC5)CC4)ccc3C2=O)C(=O)N1] | N-(5-(4-((1-(2-(2,4- dioxotetrahydropyrimidin-1(2H)-yl)-1- oxoisoindolin-5-yl)piperidin-4- yl)methyl)piperazin-1-yl)-1-((1s,4s)-4- (hydroxymethyl)cyclohexyl)-1H- benzo[d]imidazol-2-yl)-3- (trifluoromethyl)benzamide |
| GT- 0501 9 | 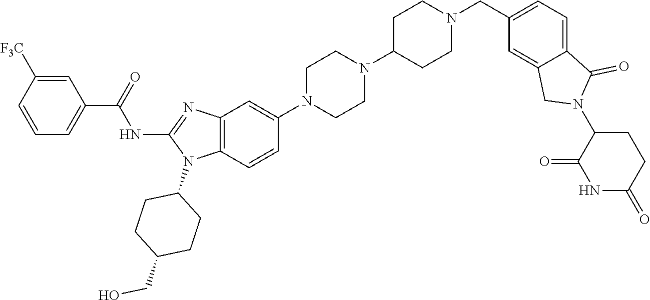 [SMILES: O=C1CCC(N2Cc3cc(CN4CCC(N5CCN(c6ccc7c(c6)nc(NC(=O)c6cccc(C(F)(F)F)c6)n7[C@H]6CC[C@@H](CO)CC6)CC5)CC4)ccc3C2=O)C(=O)N1] | N-(5-(4-(1-((2-(2,4- dioxotetrahydropyrimidin-1(2H)-yl)-1- oxoisoindolin-5-yl)methyl)piperidin-4- yl)piperazin-1-yl)-1-((1s,4s)-4- (hydroxymethyl)cyclohexyl)-1H- benzo[d]imidazol-2-yl)-3- (trifluoromethyl)benzamide |
| GT- 0531 9 | 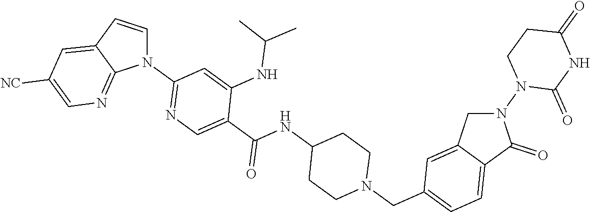 [SMILES: CC(C)Nc1cc(-n2ccc3cc(C#N)cnc32)ncc1C(=O)NC1CCN(Cc2ccc3c(c2)CN(N2CCC(=O)NC2=O)C3=O)CC1] | 6-(5-cyano-1H-pyrrolo[2,3-b]pyridin-1-yl)-N- (1-((2-(2,4-dioxotetrahydropyrimidin-1(2H)- yl)-1-oxoisoindolin-5-yl)methyl)piperidin-4- yl)-4-(isopropylamino)nicotinamide |
| GT- 0518 4 | 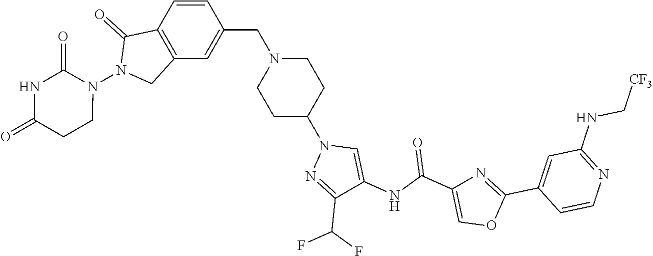 [SMILES: O=C1CCN(N2Cc3cc(CN4CCC(n5cc(NC(=O)c6coc(-c7ccnc(NCC(F)(F)F)c7)n6)c(C(F)F)n5)CC4)ccc3C2=O)C(=O)N1] | N-(3-(difluoromethyl)-1-(1-((2-(2,4- dioxotetrahydropyrimidin-1(2H)-yl)-1- oxoisoindolin-5-yl)methyl)piperidin-4-yl)- 1H-pyrazol-4-yl)-2-(2-((2,2,2- trifluoroethyl)amino)pyridin-4-yl)oxazole-4- carboxamide |
| | 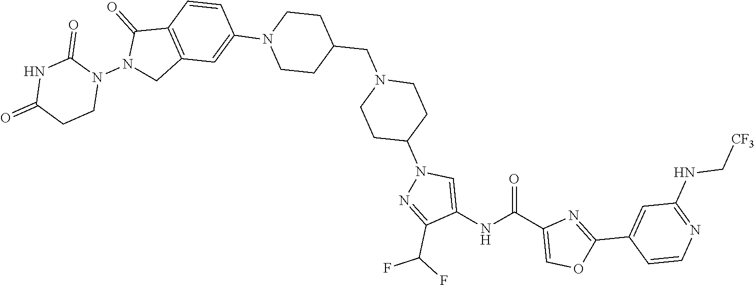 [SMILES: O=C1CCN(N2Cc3cc(N4CCC(CN5CCC(n6cc(NC(=O)c7coc(-c8ccnc(NCC(F)(F)F)c8)n7)c(C(F)F)n6)CC5)CC4)ccc3C2=O)C(=O)N1] | N-(3-(difluoromethyl)-1-(1-((1-(2-(2,4- dioxotetrahydropyrimidin-1(2H)-yl)-1- oxoisoindolin-5-yl)piperidin-4- yl)methyl)piperidin-4-yl)-1H-pyrazol-4-yl)-2- (2-((2,2,2-trifluoroethyl)amino)pyridin-4- yl)oxazole-4-carboxamide |
| | 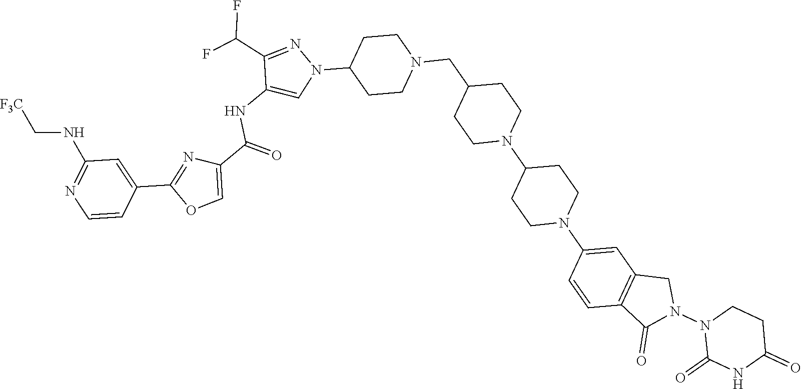 [SMILES: O=C1CCN(N2Cc3cc(N4CCC(N5CCC(CN6CCC(n7cc(NC(=O)c8coc(-c9ccnc(NCC(F)(F)F)c9)n8)c(C(F)F)n7)CC6)CC5)CC4)ccc3C2=O)C(=O)N1] | N-(3-(difluoromethyl)-1-(1-((1′-(2-(2,4- dioxotetrahydropyrimidin-1(2H)-yl)-1- oxoisoindolin-5-yl)-[1,4′-bipiperidin]-4- yl)methyl)piperidin-4-yl)-1H-pyrazol-4-yl)-2- (2-((2,2,2-trifluoroethyl)amino)pyridin-4- yl)oxazole-4-carboxamide |
| GT- 0531 8 | 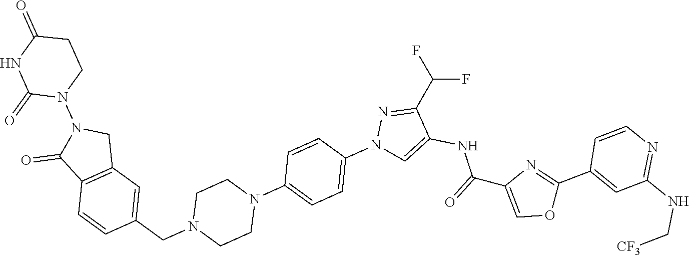 [SMILES: O=C1CCN(N2Cc3cc(CN4CCN(c5ccc(-n6cc(NC(=O)c7coc(-c8ccnc(NCC(F)(F)F)c8)n7)c(C(F)F)n6)cc5)CC4)ccc3C2=O)C(=O)N1] | N-(3-(difluoromethyl)-1-(4-(4-((2-(2,4- dioxotetrahydropyrimidin-1(2H)-yl)-1- oxoisoindolin-5-yl)methyl)piperazin-1- yl)phenyl)-1H-pyrazol-4-yl)-2-(2-((2,2,2- trifluoroethyl)amino)pyridin-4-yl)oxazole-4- carboxamide |
| GT- 0575 0 | 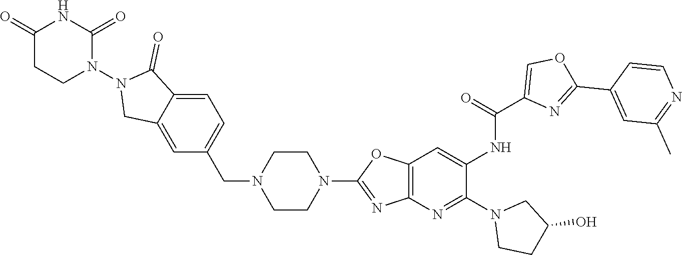 [SMILES: Cc1cc(-c2nc(C(=O)Nc3cc4oc(N5CCN(Cc6ccc7c(c6)CN(N6CCC(=O)NC6=O)C7=O)CC5)nc4nc3N3CC[C@@H](O)C3)co2)ccn1] | (R)-N-(2-(4-((2-(2,4- dioxotetrahydropyrimidin-1(2H)-yl)-1- oxoisoindolin-5-yl)methyl)piperazin-1-yl)-5- (3-hydroxypyrrolidin-1-yl)oxazolo[4,5- b]pyridin-6-yl)-2-(2-methylpyridin-4- yl)oxazole-4-carboxamide |
| GT- 0574 7 | 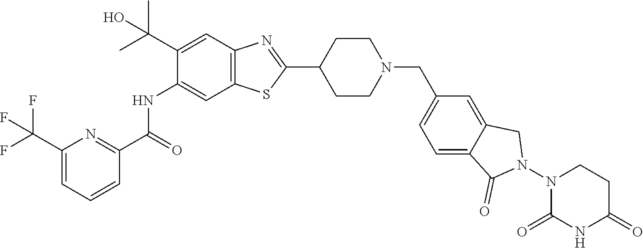 [SMILES: CC(C)(O)c1cc2nc(C3CCN(Cc4ccc5c(c4)CN(N4CCC(=O)NC4=O)C5=O)CC3)sc2cc1NC(=O)c1cccc(C(F)(F)F)n1] | N-(2-(1-((2-(2,4-dioxotetrahydropyrimidin- 1(2H)-yl)-1-oxoisoindolin-5- yl)methyl)piperidin-4-yl)-5-(2- hydroxypropan-2-yl)benzo[d]thiazol-6-yl)-6- (trifluoromethyl)picolinamide |
| GT- 0574 8 | 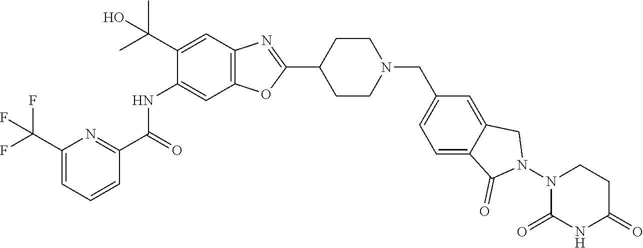 [SMILES: CC(C)(O)c1cc2nc(C3CCN(Cc4ccc5c(c4)CN(N4CCC(=O)NC4=O)C5=O)CC3)oc2cc1NC(=O)c1cccc(C(F)(F)F)n1] | N-(2-(1-((2-(2,4-dioxotetrahydropyrimidin- 1(2H)-yl)-1-oxoisoindolin-5- yl)methyl)piperidin-4-yl)-5-(2- hydroxypropan-2-yl)benzo[d]oxazol-6-yl)-6- (trifluoromethyl)picolinamide |
| GT- 0574 6 | 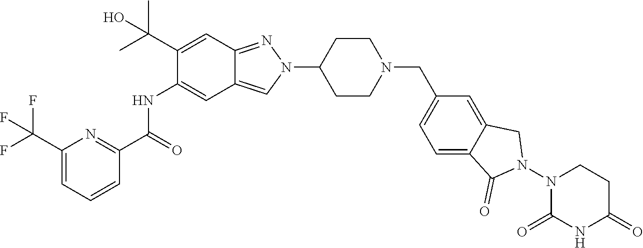 [SMILES: CC(C)(O)c1cc2nn(C3CCN(Cc4ccc5c(c4)CN(N4CCC(=O)NC4=O)C5=O)CC3)cc2cc1NC(=O)c1cccc(C(F)(F)F)n1] | N-(2-(1-((2-(2,4-dioxotetrahydropyrimidin- 1(2H)-yl)-1-oxoisoindolin-5- yl)methyl)piperidin-4-yl)-6-(2- hydroxypropan-2-yl)-2H-indazol-5-yl)-6- (trifluoromethyl)picolinamide |
| GT- 0841 8 | 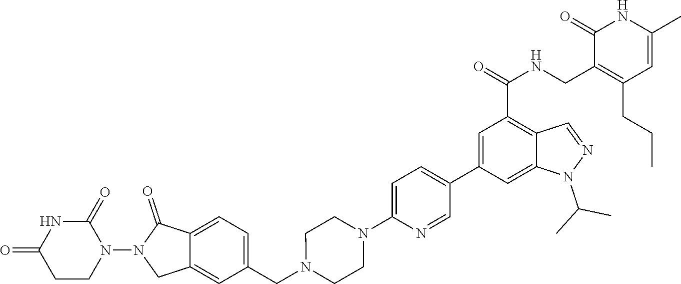 [SMILES: CCCc1cc(C)[nH]c(=O)c1CNC(=O)c1cc(-c2ccc(N3CCN(Cc4ccc5c(c4)CN(N4CCC(=O)NC4=O)C5=O)CC3)nc2)cc2c1cnn2C(C)C] | 6-(6-(4-((2-(2,4-dioxotetrahydropyrimidin- 1(2H)-yl)-1-oxoisoindolin-5- yl)methyl)piperazin-1-yl)pyridin-3-yl)-1- isopropyl-N-((6-methyl-2-oxo-4-propyl-1,2- dihydropyridin-3-yl)methyl)-1H-indazole-4- carboxamide |
| GT- 0842 0 | 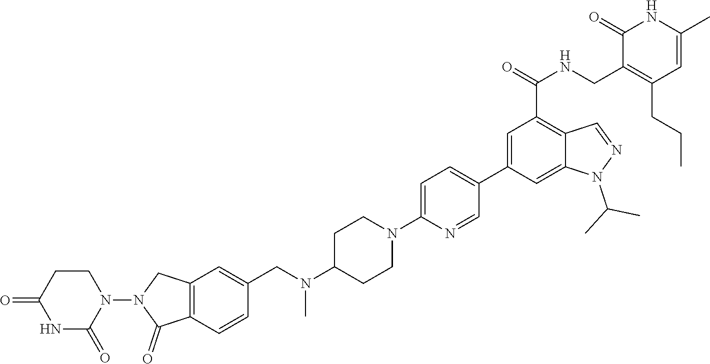 [SMILES: CCCc1cc(C)[nH]c(=O)c1CNC(=O)c1cc(-c2ccc(N3CCC(N(C)Cc4ccc5c(c4)CN(N4CCC(=O)NC4=O)C5=O)CC3)nc2)cc2c1cnn2C(C)C] | 6-(6-(4-(((2-(2,4-dioxotetrahydropyrimidin- 1(2H)-yl)-1-oxoisoindolin-5- yl)methyl)(methyl)amino)piperidin-1- yl)pyridin-3-yl)-1-isopropyl-N-((6-methyl-2- oxo-4-propyl-1,2-dihydropyridin-3- yl)methyl)-1H-indazole-4-carboxamide |
| GT- 0842 2 | 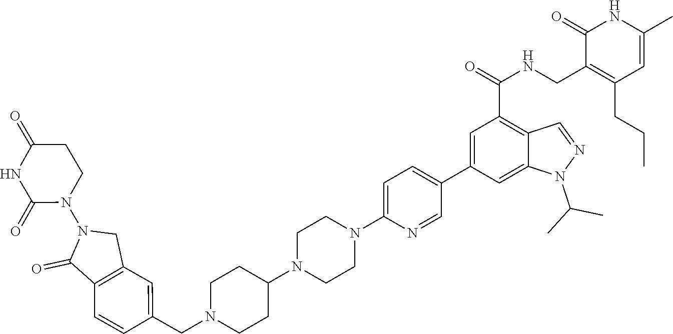 [SMILES: CCCc1cc(C)[nH]c(=O)c1CNC(=O)c1cc(-c2ccc(N3CCN(C4CCN(Cc5ccc6c(c5)CN(N5CCC(=O)NC5=O)C6=O)CC4)CC3)nc2)cc2c1cnn2C(C)C] | 6-(6-(4-(1-((2-(2,4-dioxotetrahydropyrimidin- 1(2H)-yl)-1-oxoisoindolin-5- yl)methyl)piperidin-4-yl)piperazin-1- yl)pyridin-3-yl)-1-isopropyl-N-((6-methyl-2- oxo-4-propyl-1,2-dihydropyridin-3- yl)methyl)-1H-indazole-4-carboxamide |
| GT- 0850 8 | 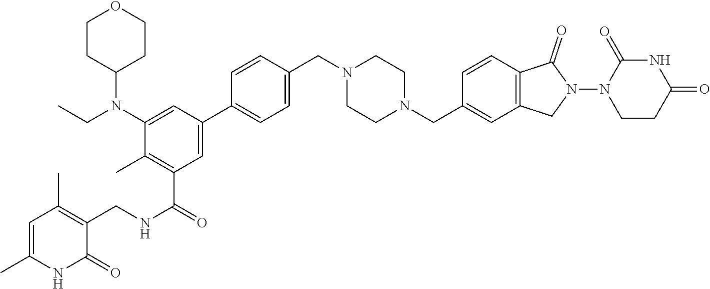 [SMILES: CCN(c1cc(-c2ccc(CN3CCN(Cc4ccc5c(c4)CN(N4CCC(=O)NC4=O)C5=O)CC3)cc2)cc(C(=O)NCc2c(C)cc(C)[nH]c2=O)c1C)C1CCOCC1] | N-((4,6-dimethyl-2-oxo-1,2-dihydropyridin- 3-yl)methyl)-4′-((4-((2-(2,4- dioxotetrahydropyrimidin-1(2H)-yl)-1- oxoisoindolin-5-yl)methyl)piperazin-1- yl)methyl)-5-(ethyl(tetrahydro-2H-pyran-4- yl)amino)-4-methyl-[1,1′-biphenyl]-3- carboxamide |
| GT- 0851 0 | 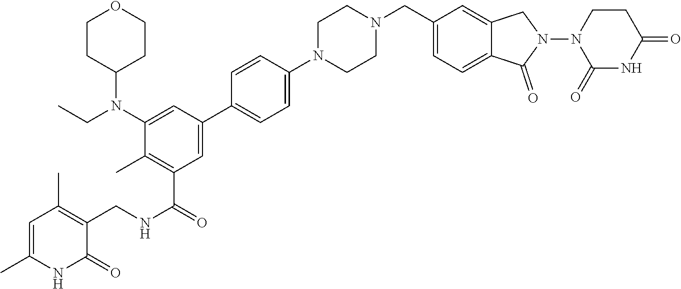 [SMILES: CCN(c1cc(-c2ccc(N3CCN(Cc4ccc5c(c4)CN(N4CCC(=O)NC4=O)C5=O)CC3)cc2)cc(C(=O)NCc2c(C)cc(C)[nH]c2=O)c1C)C1CCOCC1] | N-((4,6-dimethyl-2-oxo-1,2-dihydropyridin- 3-yl)methyl)-4′-(4-((2-(2,4- dioxotetrahydropyrimidin-1(2H)-yl)-1- oxoisoindolin-5-yl)methyl)piperazin-1-yl)-5- (ethyl(tetrahydro-2H-pyran-4-yl)amino)-4- methyl-[1,1′-biphenyl]-3-carboxamide |
| GT- 0851 2 | 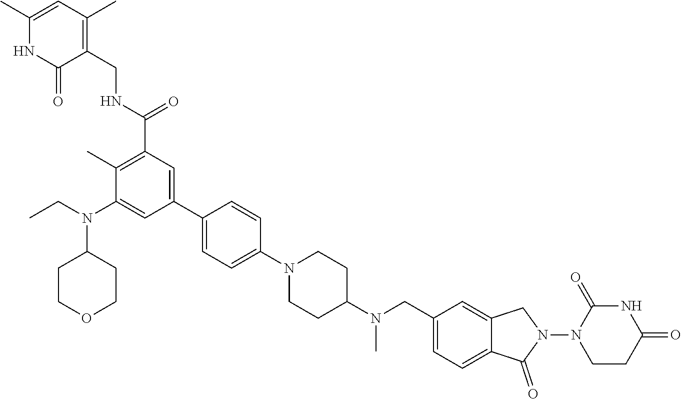 [SMILES: CCN(c1cc(-c2ccc(N3CCC(N(C)Cc4ccc5c(c4)CN(N4CCC(=O)NC4=O)C5=O)CC3)cc2)cc(C(=O)NCc2c(C)cc(C)[nH]c2=O)c1C)C1CCOCC1] | N-((4,6-dimethyl-2-oxo-1,2-dihydropyridin- 3-yl)methyl)-4′-(4-(((2-(2,4- dioxotetrahydropyrimidin-1(2H)-yl)-1- oxoisoindolin-5- yl)methyl)(methyl)amino)piperidin-1-yl)-5- (ethyl(tetrahydro-2H-pyran-4-yl)amino)-4- methyl-[1,1′-biphenyl]-3-carboxamide |
| GT- 0851 4 | 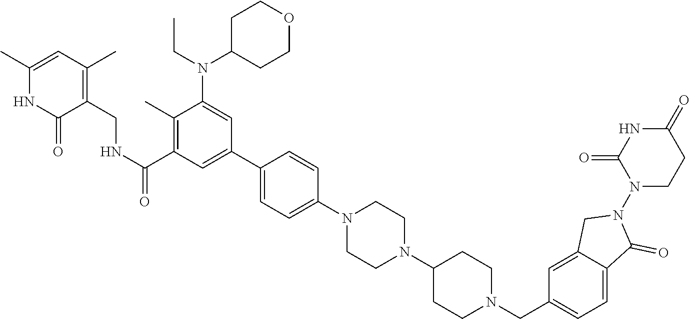 [SMILES: CCN(c1cc(-c2ccc(N3CCN(C4CCN(Cc5ccc6c(c5)CN(N5CCC(=O)NC5=O)C6=O)CC4)CC3)cc2)cc(C(=O)NCc2c(C)cc(C)[nH]c2=O)c1C)C1CCOCC1] | N-((4,6-dimethyl-2-oxo-1,2-dihydropyridin- 3-yl)methyl)-4′-(4-(1-((2-(2,4- dioxotetrahydropyrimidin-1(2H)-yl)-1- oxoisoindolin-5-yl)methyl)piperidin-4- yl)piperazin-1-yl)-5-(ethyl(tetrahydro-2H- pyran-4-yl)amino)-4-methyl-[1,1′-biphenyl]- 3-carboxamide |
| | 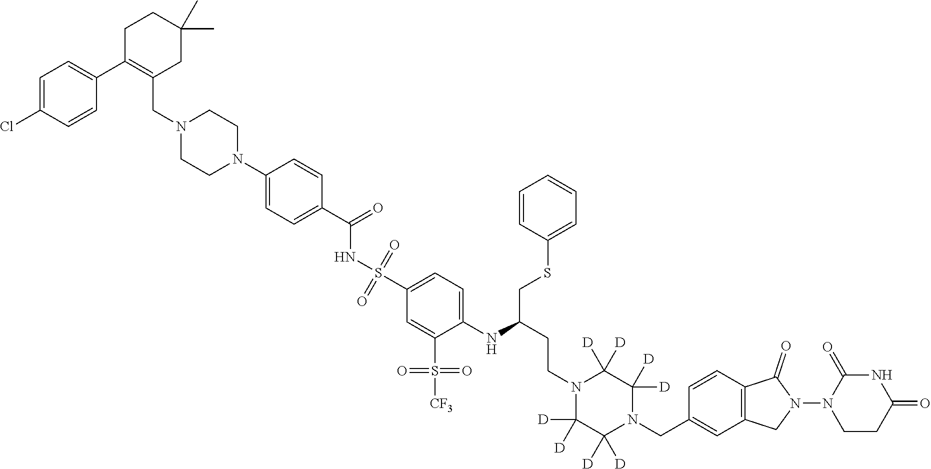 [SMILES: [2H]C1([2H])N(CC[C@H](CSc2ccccc2)Nc2ccc(S(=O)(=O)NC(=O)c3ccc(N4CCN(CC5=C(c6ccc(Cl)cc6)CCC(C)(C)C5)CC4)cc3)cc2S(=O)(=O)C(F)(F)F)C([2H])([2H])C([2H])([2H])N(Cc2ccc3c(c2)CN(N2CCC(=O)NC2=O)C3=O)C1([2H])[2H]] | (R)-4-(4-((4′-chloro-4,4-dimethyl-3,4,5,6- tetrahydro-[1,1′-biphenyl]-2- yl)methyl)piperazin-1-yl)-N-((4-((4-(4-((2- (2,4-dioxotetrahydropyrimidin-1(2H)-yl)-1- oxoisoindolin-5-yl)methyl)piperazin-1-yl- 2,2,3,3,5,5,6,6-d₈)-1-(phenylthio)butan-2- yl)amino)-3- ((trifluoromethyl)sulfonyl)phenyl)sulfonyl) benzamide |
| | 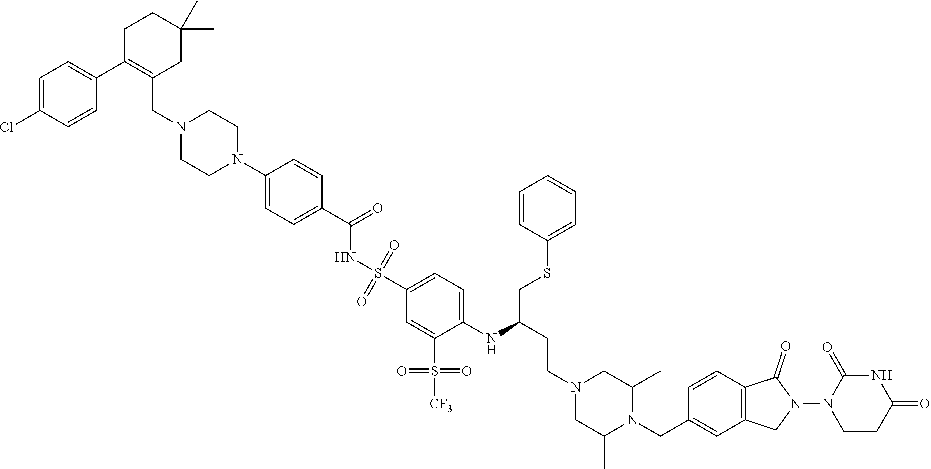 [SMILES: CC1CN(CC[C@H](CSc2ccccc2)Nc2ccc(S(=O)(=O)NC(=O)c3ccc(N4CCN(CC5=C(c6ccc(Cl)cc6)CCC(C)(C)C5)CC4)cc3)cc2S(=O)(=O)C(F)(F)F)CC(C)N1Cc1ccc2c(c1)CN(N1CCC(=O)NC1=O)C2=O] | 4-(4-((4′-chloro-4,4-dimethyl-3,4,5,6- tetrahydro-[1,1′-biphenyl]-2- yl)methyl)piperazin-1-yl)-N-((4-(((2R)-4-(4- ((2-(2,4-dioxotetrahydropyrimidin-1(2H)-yl)- 1-oxoisoindolin-5-yl)methyl)-3,5- dimethylpiperazin-1-yl)-1-(phenylthio)butan- 2-yl)amino)-3- ((trifluoromethyl)sulfonyl)phenyl)sulfonyl) benzamide |
| | 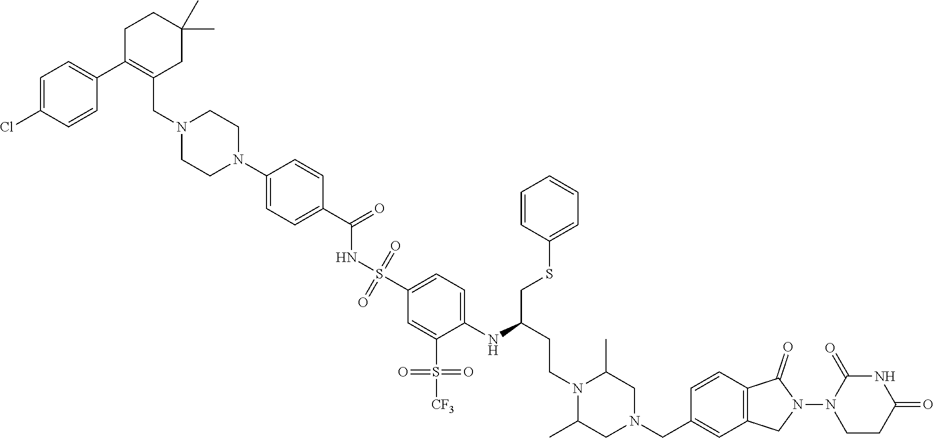 [SMILES: CC1CN(Cc2ccc3c(c2)CN(N2CCC(=O)NC2=O)C3=O)CC(C)N1CC[C@H](CSc1ccccc1)Nc1ccc(S(=O)(=O)NC(=O)c2ccc(N3CCN(CC4=C(c5ccc(Cl)cc5)CCC(C)(C)C4)CC3)cc2)cc1S(=O)(=O)C(F)(F)F] | 4-(4-((4′-chloro-4,4-dimethyl-3,4,5,6- tetrahydro-[1,1′-biphenyl]-2- yl)methyl)piperazin-1-yl)-N-((4-(((2R)-4-(4- ((2-(2,4-dioxotetrahydropyrimidin-1(2H)-yl)- 1-oxoisoindolin-5-yl)methyl)-2,6- dimethylpiperazin-1-yl)-1-(phenylthio)butan- 2-yl)amino)-3- ((trifluoromethyl)sulfonyl)phenyl)sulfonyl) benzamide |
| | 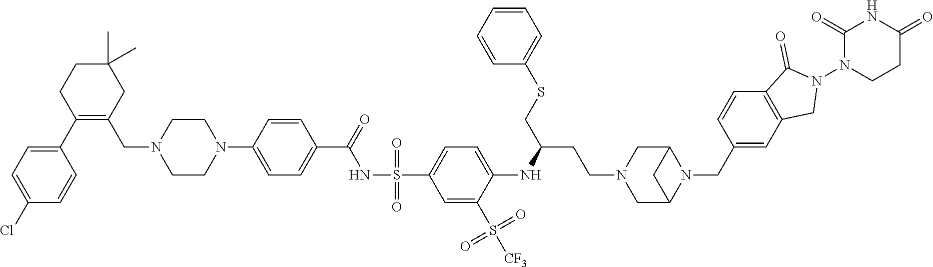 [SMILES: CC1(C)CCC(c2ccc(Cl)cc2)=C(CN2CCN(c3ccc(C(=O)NS(=O)(=O)c4ccc(N[C@H](CCN5CC6CC(C5)N6Cc5ccc6c(c5)CN(N5CCC(=O)NC5=O)C6=O)CSc5ccccc5)c(S(=O)(=O)C(F)(F)F)c4)cc3)CC2)C1] | 4-(4-((4′-chloro-4,4-dimethyl-3,4,5,6- tetrahydro-[1,1′-biphenyl]-2- yl)methyl)piperazin-1-yl)-N-((4-(((2R)-4-(6- ((2-(2,4-dioxotetrahydropyrimidin-1(2H)-yl)- 1-oxoisoindolin-5-yl)methyl)-3,6- diazabicyclo[3.1.1]heptan-3-yl)-1- (phenylthio)butan-2-yl)amino)-3- ((trifluoromethyl)sulfonyl)phenyl)sulfonyl) benzamide |
| | 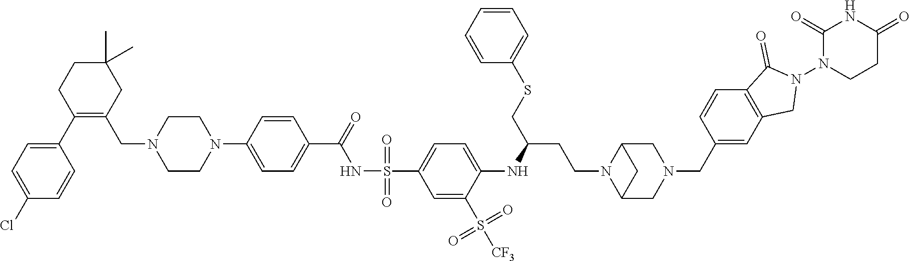 [SMILES: CC1(C)CCC(c2ccc(Cl)cc2)=C(CN2CCN(c3ccc(C(=O)NS(=O)(=O)c4ccc(N[C@H](CCN5C6CC5CN(Cc5ccc7c(c5)CN(N5CCC(=O)NC5=O)C7=O)C6)CSc5ccccc5)c(S(=O)(=O)C(F)(F)F)c4)cc3)CC2)C1] | 4-(4-((4′-chloro-4,4-dimethyl-3,4,5,6- tetrahydro-[1,1′-biphenyl]-2- yl)methyl)piperazin-1-yl)-N-((4-(((2R)-4-(3- ((2-(2,4-dioxotetrahydropyrimidin-1(2H)-yl)- 1-oxoisoindolin-5-yl)methyl)-3,6- diazabicyclo[3.1.1]heptan-6-yl)-1- (phenylthio)butan-2-yl)amino)-3- ((trifluoromethyl)sulfonyl)phenyl)sulfonyl) benzamide |
| GT- 0781 3 | 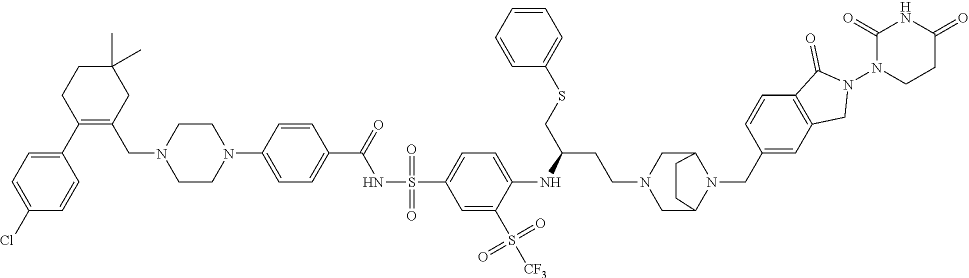 | 4-(4-((4′-chloro-4,4-dimethyl-3,4,5,6- tetrahydro-[1,1′-biphenyl]-2- yl)methyl)piperazin-1-yl)-N-((4-(((2R)-4-(8- ((2-(2,4-dioxotetrahydropyrimidin-1(2H)-yl)- 1-oxoisoindolin-5-yl)methyl)-3,8- diazabicyclo[3.2.1]octan-3-yl)-1- (phenylthio)butan-2-yl)amino)-3- ((trifluoromethyl)sulfonyl)phenyl)sulfonyl) benzamide |
| GT- 0781 4 | 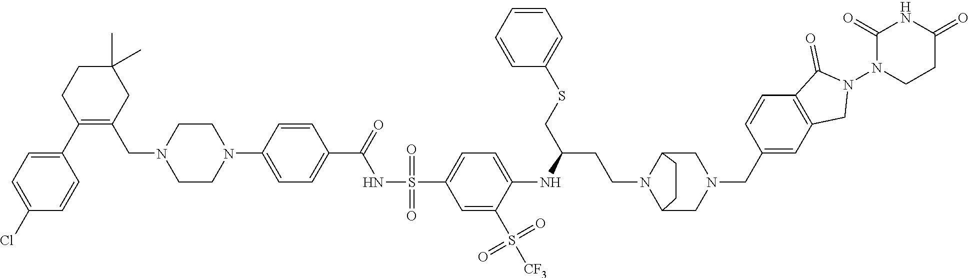 | 4-(4-((4′-chloro-4,4-dimethyl-3,4,5,6- tetrahydro-[1,1′-biphenyl]-2- yl)methyl)piperazin-1-yl)-N-((4-(((2R)-4-(3- ((2-(2,4-dioxotetrahydropyrimidin-1(2H)-yl)- 1-oxoisoindolin-5-yl)methyl)-3,8- diazabicyclo[3.2.1]octan-8-yl)-1- (phenylthio)butan-2-yl)amino)-3- ((trifluoromethyl)sulfonyl)phenyl)sulfonyl) benzamide |
| GT- 0781 2 | 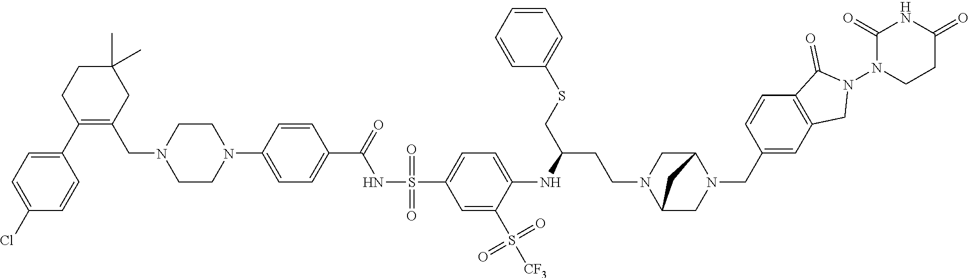 | 4-(4-((4′-chloro-4,4-dimethyl-3,4,5,6- tetrahydro-[1,1′-biphenyl]-2- yl)methyl)piperazin-1-yl)-N-((4-(((R)-4- ((1R,4R)-5-((2-(2,4- dioxotetrahydropyrimidin-1(2H)-yl)-1- oxoisoindolin-5-yl)methyl)-2,5- diazabicyclo[2.2.1]heptan-2-yl)-1- (phenylthio)butan-2-yl)amino)-3- ((trifluoromethyl)sulfonyl)phenyl)sulfonyl) benzamide |
| | 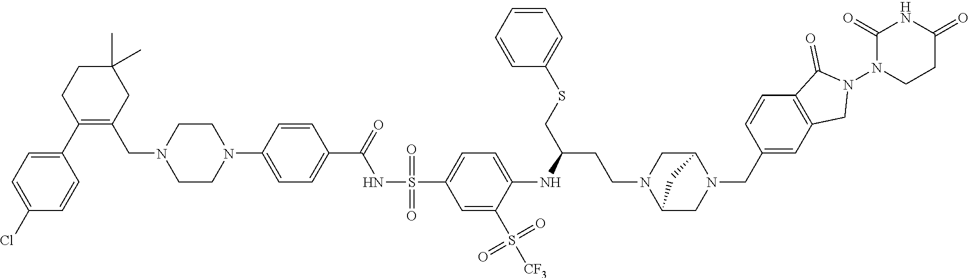 | 4-(4-((4′-chloro-4,4-dimethyl-3,4,5,6- tetrahydro-[1,1′-biphenyl]-2- yl)methyl)piperazin-1-yl)-N-((4-(((R)-4- ((1S,4S)-5-((2-(2,4- dioxotetrahydropyrimidin-1(2H)-yl)-1- oxoisoindolin-5-yl)methyl)-2,5- diazabicyclo[2.2.1]heptan-2-yl)-1- (phenylthio)butan-2-yl)amino)-3- ((trifluoromethyl)sulfonyl)phenyl)sulfonyl) benzamide |
| GT- 0781 1 | 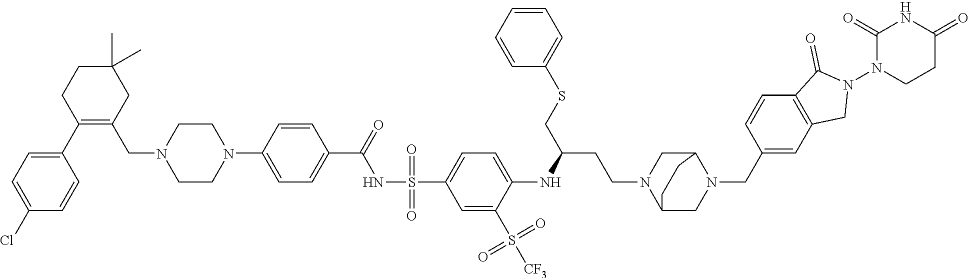 | 4-(4-((4′-chloro-4,4-dimethyl-3,4,5,6- tetrahydro-[1,1′-biphenyl]-2- yl)methyl)piperazin-1-yl)-N-((4-(((2R)-4-(5- ((2-(2,4-dioxotetrahydropyrimidin-1(2H)-yl)- 1-oxoisoindolin-5-yl)methyl)-2,5- diazabicyclo[2.2.2]octan-2-yl)-1- (phenylthio)butan-2-yl)amino)-3- ((trifluoromethyl)sulfonyl)phenyl)sulfonyl) benzamide |
| | 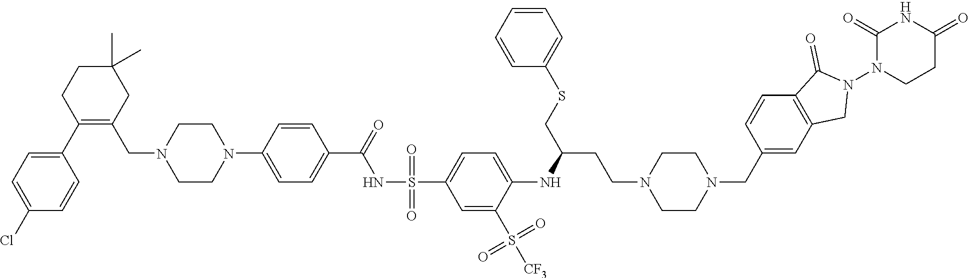 | (R)-4-(4-((4′-chloro-4,4-dimethyl-3,4,5,6- tetrahydro-[1,1′-biphenyl]-2- yl)methyl)piperazin-1-yl)-N-((4-((4-(4-((2- (2,4-dioxotetrahydropyrimidin-1(2H)-yl)-1- oxoisoindolin-5-yl)methyl)piperazin-1-yl)-1- (phenylthio)butan-2-yl)amino)-3- ((trifluoromethyl)sulfonyl)phenyl)sulfonyl) benzamide |
| | 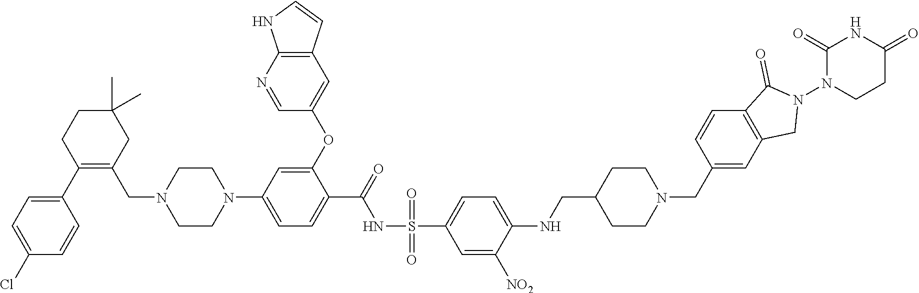 | 2-((1H-pyrrolo[2,3-b]pyridin-5-yl)oxy)-4-(4- ((4′-chloro-5,5-dimethyl-3,4,5,6-tetrahydro- [1,1′-biphenyl]-2-yl)methyl)piperazin-1-yl)- N-((4-(((1-((2-(2,4-dioxotetrahydropyrimidin- 1(2H)-yl)-1-oxoisoindolin-5- yl)methyl)piperidin-4-yl)methyl)amino)-3- nitrophenyl)sulfonyl)benzamide |
| | 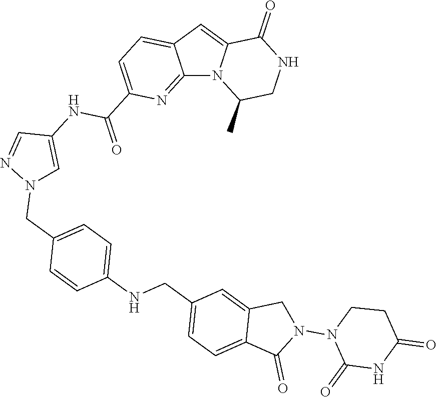 [SMILES: C[C@@H]1CNC(=O)c2cc3ccc(C(=O)Nc4cnn(Cc5ccc(NCc6ccc7c(c6)CN(N6CCC(=O)NC6=O)C7=O)cc5)c4)nc3n21] | (R)-N-(1-(4-(((2-(2,4- dioxotetrahydropyrimidin-1(2H)-yl)-1- oxoisoindolin-5-yl)methyl)amino)benzyl)- 1H-pyrazol-4-yl)-9-methyl-6-oxo-6,7,8,9- tetrahydropyrido[3′,2′:4,5]pyrrolo[1,2- a]pyrazine-2-carboxamide |
| GT- 0814 9 | 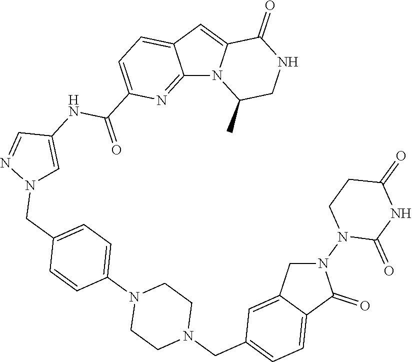 [SMILES: C[C@@H]1CNC(=O)c2cc3ccc(C(=O)Nc4cnn(Cc5ccc(N6CCN(Cc7ccc8c(c7)CN(N7CCC(=O)NC7=O)C8=O)CC6)cc5)c4)nc3n21] | (R)-N-(1-(4-(4-((2-(2,4- dioxotetrahydropyrimidin-1(2H)-yl)-1- oxoisoindolin-5-yl)methyl)piperazin-1- yl)benzyl)-1H-pyrazol-4-yl)-9-methyl-6-oxo- 6,7,8,9-tetrahydropyrido[3′,2′:4,5]pyrrolo[1,2- a]pyrazine-2-carboxamide |
| | 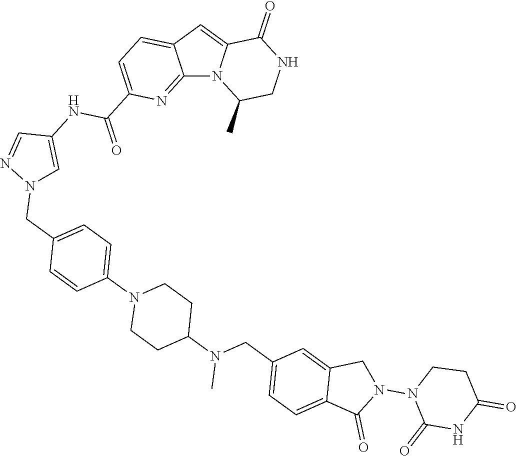 [SMILES: C[C@@H]1CNC(=O)c2cc3ccc(C(=O)Nc4cnn(Cc5ccc(N6CCC(N(C)Cc7ccc8c(c7)CN(N7CCC(=O)NC7=O)C8=O)CC6)cc5)c4)nc3n21] | (R)-N-(1-(4-(4-(((2-(2,4- dioxotetrahydropyrimidin-1(2H)-yl)-1- oxoisoindolin-5- yl)methyl)(methyl)amino)piperidin-1- yl)benzyl)-1H-pyrazol-4-yl)-9-methyl-6-oxo- 6,7,8,9-tetrahydropyrido[3′,2′:4,5]pyrrolo[1,2- a]pyrazine-2-carboxamide |
| | 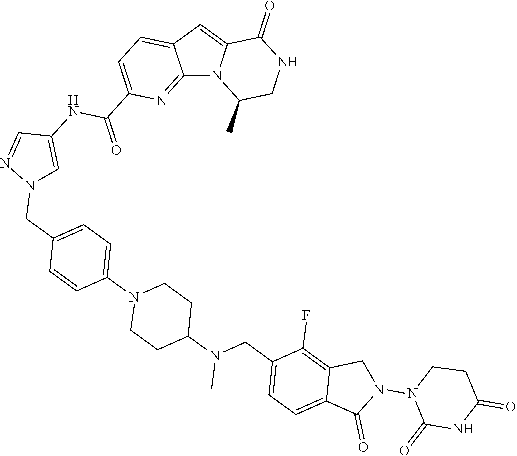 [SMILES: C[C@@H]1CNC(=O)c2cc3ccc(C(=O)Nc4cnn(Cc5ccc(N6CCC(N(C)Cc7ccc8c(c7F)CN(N7CCC(=O)NC7=O)C8=O)CC6)cc5)c4)nc3n21] | (R)-N-(1-(4-(4-(((2-(2,4- dioxotetrahydropyrimidin-1(2H)-yl)-4-fluoro- 1-oxoisoindolin-5- yl)methyl)(methyl)amino)piperidin-1- yl)benzyl)-1H-pyrazol-4-yl)-9-methyl-6-oxo- 6,7,8,9-tetrahydropyrido[3′,2′:4,5]pyrrolo[1,2- a]pyrazine-2-carboxamide |
| GT- 0815 1 | 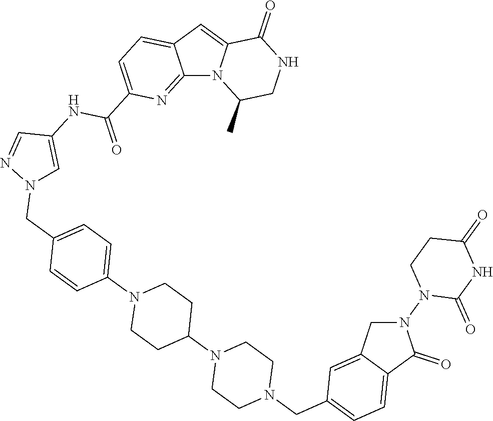 [SMILES: C[C@@H]1CNC(=O)c2cc3ccc(C(=O)Nc4cnn(Cc5ccc(N6CCC(N7CCN(Cc8ccc9c(c8)CN(N8CCC(=O)NC8=O)C9=O)CC7)CC6)cc5)c4)nc3n21] | (R)-N-(1-(4-(4-(4-((2-(2,4- dioxotetrahydropyrimidin-1(2H)-yl)-1- oxoisoindolin-5-yl)methyl)piperazin-1- yl)piperidin-1-yl)benzyl)-1H-pyrazol-4-yl)-9- methyl-6-oxo-6,7,8,9- tetrahydropyrido[3′,2′:4,5]pyrrolo[1,2- a]pyrazine-2-carboxamide |
| GT- 0495 2 | 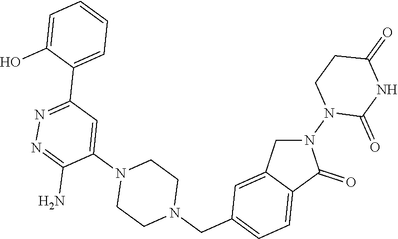 [SMILES: Nc1nnc(-c2ccccc2O)cc1N1CCN(Cc2ccc3c(c2)CN(N2CCC(=O)NC2=O)C3=O)CC1] | 1-(5-((4-(3-amino-6-(2- hydroxyphenyl)pyridazin-4-yl)piperazin-1- yl)methyl)-1-oxoisoindolin-2- yl)dihydropyrimidine-2,4(1H,3H)-dione |
| GT- 0506 1 | 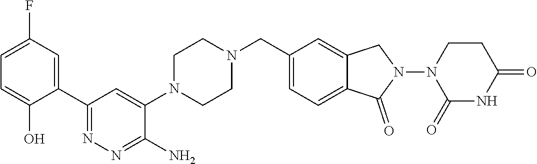 [SMILES: Nc1nnc(-c2cc(F)ccc2O)cc1N1CCN(Cc2ccc3c(c2)CN(N2CCC(=O)NC2=O)C3=O)CC1] | 1-(5-((4-(3-amino-6-(5-fluoro-2- hydroxyphenyl)pyridazin-4-yl)piperazin-1- yl)methyl)-1-oxoisoindolin-2- yl)dihydropyrimidine-2,4(1H,3H)-dione |
| GT- 0482 4 | 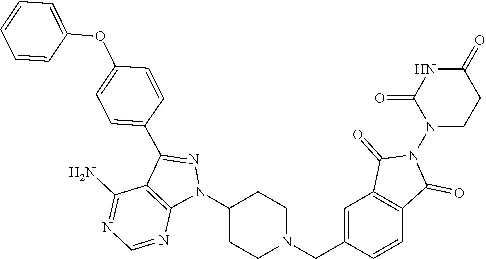 [SMILES: Nc1ncnc2c1c(-c1ccc(Oc3ccccc3)cc1)nn2C1CCN(Cc2ccc3c(c2)C(=O)N(N2CCC(=O)NC2=O)C3=O)CC1] | 5-((4-(4-amino-3-(4-phenoxyphenyl)-1H- pyrazolo[3,4-d]pyrimidin-1-yl)piperidin-1- yl)methyl)-2-(2,4-dioxotetrahydropyrimidin- 1(2H)-yl)isoindoline-1,3-dione |
| GT- 0482 4 | 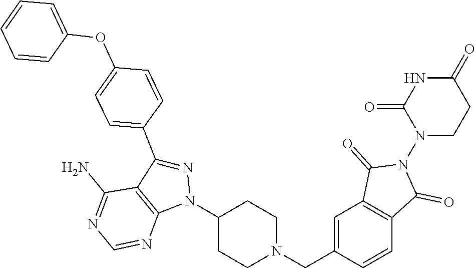 [SMILES: Nc1ncnc2c1c(-c1ccc(Oc3ccccc3)cc1)nn2C1CCN(Cc2ccc3c(c2)C(=O)N(N2CCC(=O)NC2=O)C3=O)CC1] | 5-((4-(4-amino-3-(4-phenoxyphenyl)-1H- pyrazolo[3,4-d]pyrimidin-1-yl)piperidin-1- yl)methyl)-2-(2,4-dioxotetrahydropyrimidin- 1(2H)-yl)isoindoline-1,3-dione |
| GT- 0507 6 | 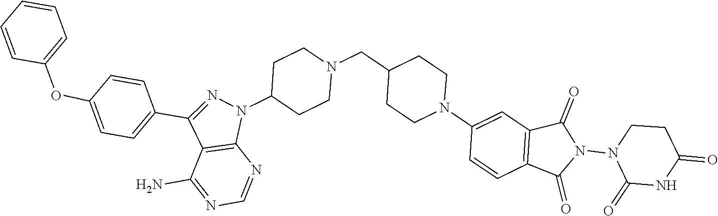 [SMILES: Nc1ncnc2c1c(-c1ccc(Oc3ccccc3)cc1)nn2C1CCN(CC2CCN(c3ccc4c(c3)C(=O)N(N3CCC(=O)NC3=O)C4=O)CC2)CC1] | 5-(4-((4-(4-amino-3-(4-phenoxyphenyl)-1H- pyrazolo[3,4-d]pyrimidin-1-yl)piperidin-1- yl)methyl)piperidin-1-yl)-2-(2,4- dioxotetrahydropyrimidin-1(2H)- yl)isoindoline-1,3-dione |
| GT- 0507 7 | 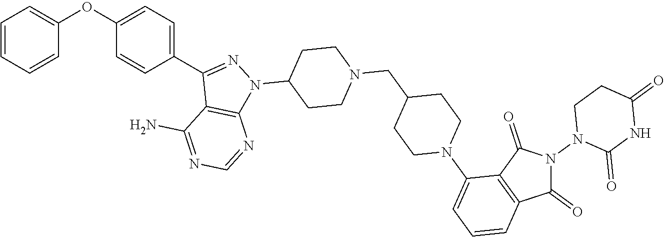 [SMILES: Nc1ncnc2c1c(-c1ccc(Oc3ccccc3)cc1)nn2C1CCN(CC2CCN(c3cccc4c3C(=O)N(N3CCC(=O)NC3=O)C4=O)CC2)CC1] | 4-(4-((4-(4-amino-3-(4-phenoxyphenyl)-1H- pyrazolo[3,4-d]pyrimidin-1-yl)piperidin-1- yl)methyl)piperidin-1-yl)-2-(2,4- dioxotetrahydropyrimidin-1(2H)- yl)isoindoline-1,3-dione |
| GT- 0509 6 | 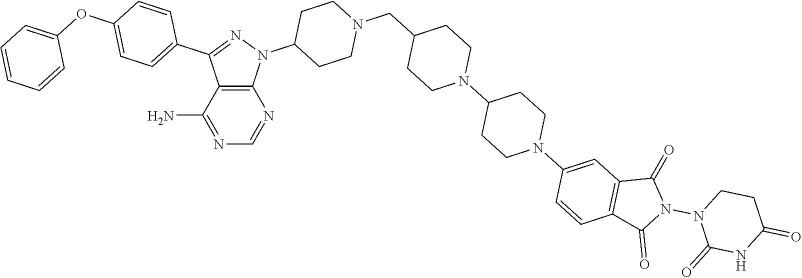 | 5-(4-((4-(4-amino-3-(4-phenoxyphenyl)-1H- pyrazolo[3,4-d]pyrimidin-1-yl)piperidin-1- yl)methyl)-[1,4′-bipiperidin]-1′-yl)-2-(2,4- dioxotetrahydropyrimidin-1(2H)- yl)isoindoline-1,3-dione |
| GT- 0482 3 | 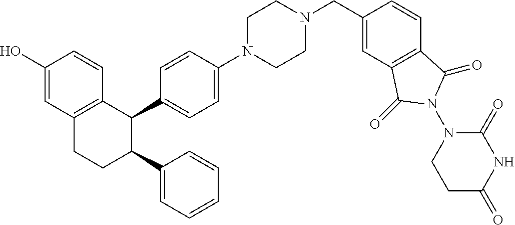 [SMILES: O=C1CCN(N2C(=O)c3ccc(CN4CCN(c5ccc([C@@H]6c7ccc(O)cc7CC[C@@H]6c6ccccc6)cc5)CC4)cc3C2=O)C(=O)N1] | 2-(2,4-dioxotetrahydropyrimidin-1(2H)-yl)-5- ((4-(4-((1R,2S)-6-hydroxy-2-phenyl-1,2,3,4- tetrahydronaphthalen-1-yl)phenyl)piperazin- 1-yl)methyl)isoindoline-1,3-dione |
| GT- 0477 8 | 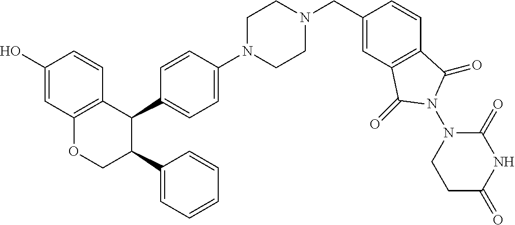 [SMILES: O=C1CCN(N2C(=O)c3ccc(CN4CCN(c5ccc([C@@H]6c7ccc(O)cc7OC[C@@H]6c6ccccc6)cc5)CC4)cc3C2=O)C(=O)N1] | 2-(2,4-dioxotetrahydropyrimidin-1(2H)-yl)-5- ((4-(4-((3S,4R)-7-hydroxy-3-phenylchroman- 4-yl)phenyl)piperazin-1- yl)methyl)isoindoline-1,3-dione |
| GT- 0530 1 | 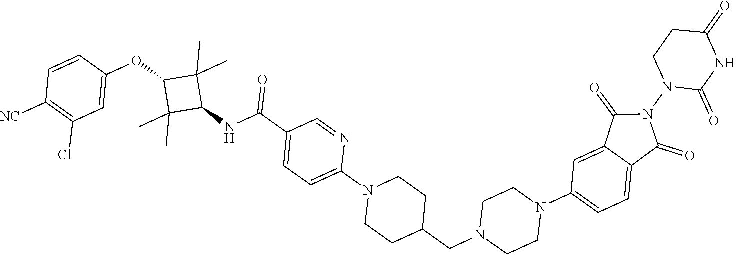 [SMILES: CC1(C)[C@H](NC(=O)c2ccc(N3CCC(CN4CCN(c5ccc6c(c5)C(=O)N(N5CCC(=O)NC5=O)C6=O)CC4)CC3)nc2)C(C)(C)[C@H]1Oc1ccc(C#N)c(Cl)c1] | N-((1r,3r)-3-(3-chloro-4-cyanophenoxy)- 2,2,4,4-tetramethylcyclobutyl)-6-(4-((4-(2- (2,4-dioxotetrahydropyrimidin-1(2H)-yl)-1,3- dioxoisoindolin-5-yl)piperazin-1- yl)methyl)piperidin-1-yl)nicotinamide |
| GT- 0530 2 | 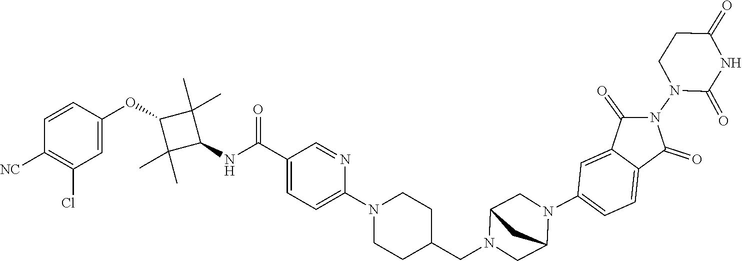 [SMILES: CC1(C)[C@H](NC(=O)c2ccc(N3CCC(CN4C[C@@H]5C[C@H]4CN5c4ccc5c(c4)C(=O)N(N4CCC(=O)NC4=O)C5=O)CC3)nc2)C(C)(C)[C@H]1Oc1ccc(C#N)c(Cl)c1] | N-((1r,3S)-3-(3-chloro-4-cyanophenoxy)- 2,2,4,4-tetramethylcyclobutyl)-6-(4- (((1S,4S)-5-(2-(2,4- dioxotetrahydropyrimidin-1(2H)-yl)-1,3- dioxoisoindolin-5-yl)-2,5- diazabicyclo[2.2.1]heptan-2- yl)methyl)piperidin-1-yl)nicotinamide |
| GT- 0530 3 | 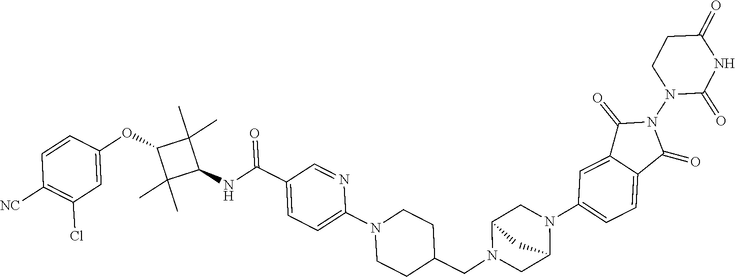 [SMILES: CC1(C)[C@H](NC(=O)c2ccc(N3CCC(CN4C[C@H]5C[C@@H]4CN5c4ccc5c(c4)C(=O)N(N4CCC(=O)NC4=O)C5=O)CC3)nc2)C(C)(C)[C@H]1Oc1ccc(C#N)c(Cl)c1] | N-((1r,3R)-3-(3-chloro-4-cyanophenoxy)- 2,2,4,4-tetramethylcyclobutyl)-6-(4- (((1R,4R)-5-(2-(2,4- dioxotetrahydropyrimidin-1(2H)-yl)-1,3- dioxoisoindolin-5-yl)-2,5- diazabicyclo[2.2.1]heptan-2- yl)methyl)piperidin-1-yl)nicotinamide |
| GT- 0530 4 | 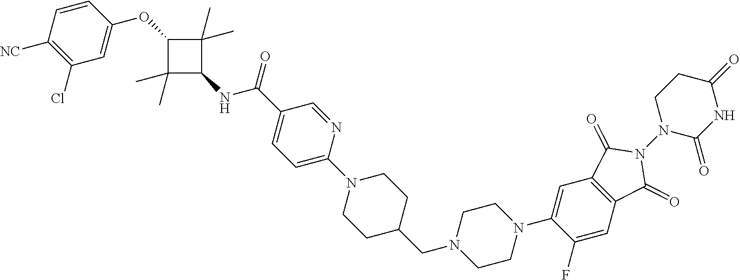 | N-((1r,3r)-3-(3-chloro-4-cyanophenoxy)- 2,2,4,4-tetramethylcyclobutyl)-6-(4-((4-(2- (2,4-dioxotetrahydropyrimidin-1(2H)-yl)-6- fluoro-1,3-dioxoisoindolin-5-yl)piperazin-1- yl)methyl)piperidin-1-yl)nicotinamide |
| GT- 0530 5 | 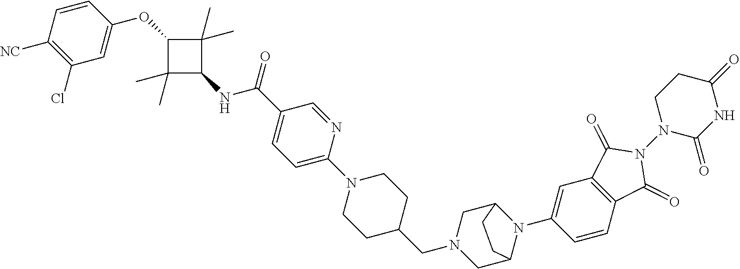 | N-((1r,3r)-3-(3-chloro-4-cyanophenoxy)- 2,2,4,4-tetramethylcyclobutyl)-6-(4-((8-(2- (2,4-dioxotetrahydropyrimidin-1(2H)-yl)-1,3- dioxoisoindolin-5-yl)-3,8- diazabicyclo[3.2.1]octan-3- yl)methyl)piperidin-1-yl)nicotinamide |
| GT- 0530 6 | 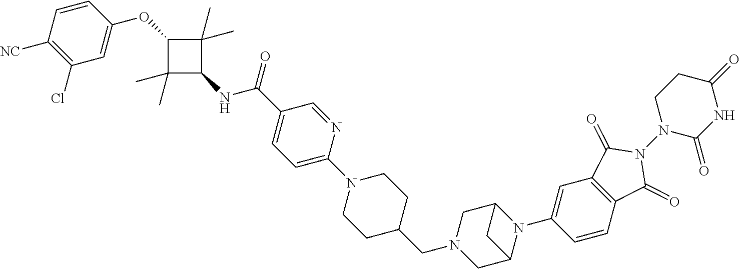 | N-((1r,3r)-3-(3-chloro-4-cyanophenoxy)- 2,2,4,4-tetramethylcyclobutyl)-6-(4-((6-(2- (2,4-dioxotetrahydropyrimidin-1(2H)-yl)-1,3- dioxoisoindolin-5-yl)-3,6- diazabicyclo[3.1.1]heptan-3- yl)methyl)piperidin-1-yl)nicotinamide |
| GT- 0534 5 | 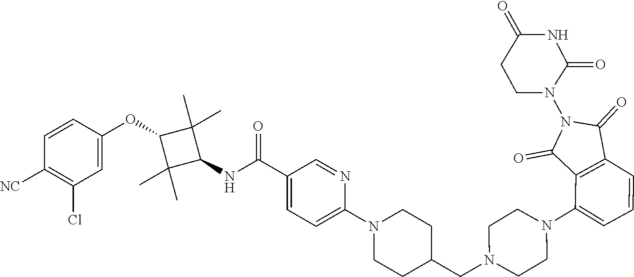 [SMILES: CC1(C)[C@H](NC(=O)c2ccc(N3CCC(CN4CCN(c5cccc6c5C(=O)N(N5CCC(=O)NC5=O)C6=O)CC4)CC3)nc2)C(C)(C)[C@H]1Oc1ccc(C#N)c(Cl)c1] | N-((1r,3r)-3-(3-chloro-4-cyanophenoxy)- 2,2,4,4-tetramethylcyclobutyl)-6-(4-((4-(2- (2,4-dioxotetrahydropyrimidin-1(2H)-yl)-1,3- dioxoisoindolin-4-yl)piperazin-1- yl)methyl)piperidin-1-yl)nicotinamide |
| GT- 0534 6 | 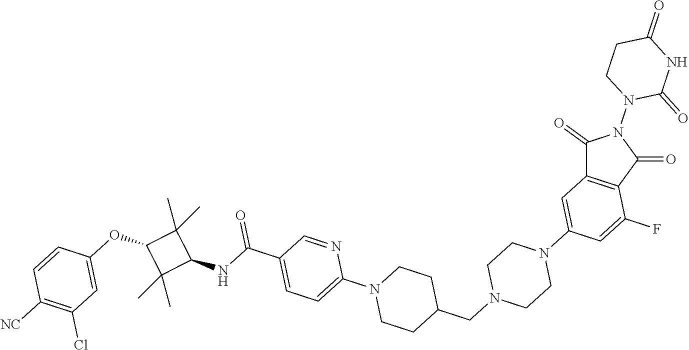 [SMILES: CC1(C)[C@H](NC(=O)c2ccc(N3CCC(CN4CCN(c5cc(F)c6c(c5)C(=O)N(N5CCC(=O)NC5=O)C6=O)CC4)CC3)nc2)C(C)(C)[C@H]1Oc1ccc(C#N)c(Cl)c1] | N-((1r,3r)-3-(3-chloro-4-cyanophenoxy)- 2,2,4,4-tetramethylcyclobutyl)-6-(4-((4-(2- (2,4-dioxotetrahydropyrimidin-1(2H)-yl)-7- fluoro-1,3-dioxoisoindolin-5-yl)piperazin-1- yl)methyl)piperidin-1-yl)nicotinamide |
| GT- 0534 7 | 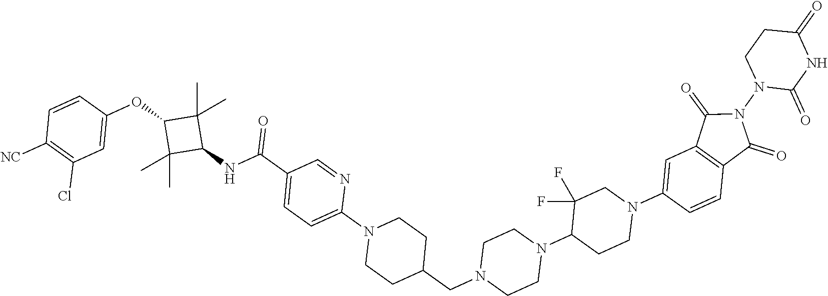 [SMILES: CC1(C)[C@H](NC(=O)c2ccc(N3CCC(CN4CCN(C5CCN(c6ccc7c(c6)C(=O)N(N6CCC(=O)NC6=O)C7=O)CC5(F)F)CC4)CC3)nc2)C(C)(C)[C@H]1Oc1ccc(C#N)c(Cl)c1] | N-((1r,3r)-3-(3-chloro-4-cyanophenoxy)- 2,2,4,4-tetramethylcyclobutyl)-6-(4-((4-(1-(2- (2,4-dioxotetrahydropyrimidin-1(2H)-yl)-1,3- dioxoisoindolin-5-yl)-3,3-difluoropiperidin-4- yl)piperazin-1-yl)methyl)piperidin-1- yl)nicotinamide |
| GT- 0522 5 | 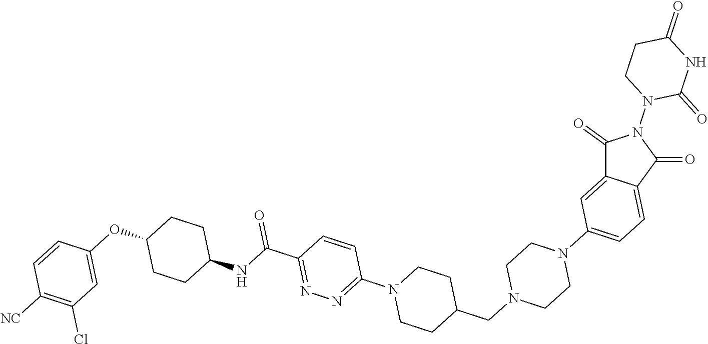 [SMILES: N#Cc1ccc(O[C@H]2CC[C@H](NC(=O)c3ccc(N4CCC(CN5CCN(c6ccc7c(c6)C(=O)N(N6CCC(=O)NC6=O)C7=O)CC5)CC4)nn3)CC2)cc1Cl] | N-((1r,4r)-4-(3-chloro-4- cyanophenoxy)cyclohexyl)-6-(4-((4-(2-(2,4- dioxotetrahydropyrimidin-1(2H)-yl)-1,3- dioxoisoindolin-5-yl)piperazin-1- yl)methyl)piperidin-1-yl)pyridazine-3- carboxamide |
| GT- 0522 6 | 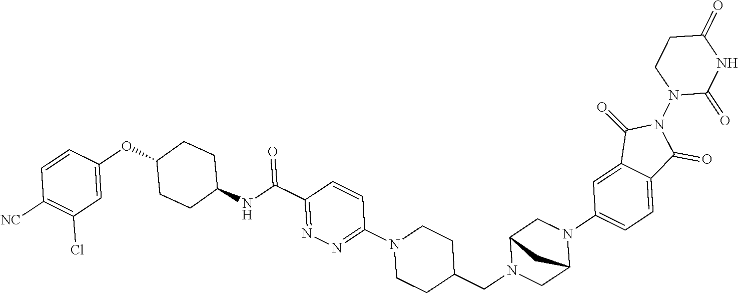 [SMILES: N#Cc1ccc(O[C@H]2CC[C@H](NC(=O)c3ccc(N4CCC(CN5C[C@@H]6C[C@H]5CN6c5ccc6c(c5)C(=O)N(N5CCC(=O)NC5=O)C6=O)CC4)nn3)CC2)cc1Cl] | N-((1r,4r)-4-(3-chloro-4- cyanophenoxy)cyclohexyl)-6-(4-(((1S,4S)-5- (2-(2,4-dioxotetrahydropyrimidin-1(2H)-yl)- 1,3-dioxoisoindolin-5-yl)-2,5- diazabicyclo[2.2.1]heptan-2- yl)methyl)piperidin-1-yl)pyridazine-3- carboxamide |
| GT- 0522 7 | 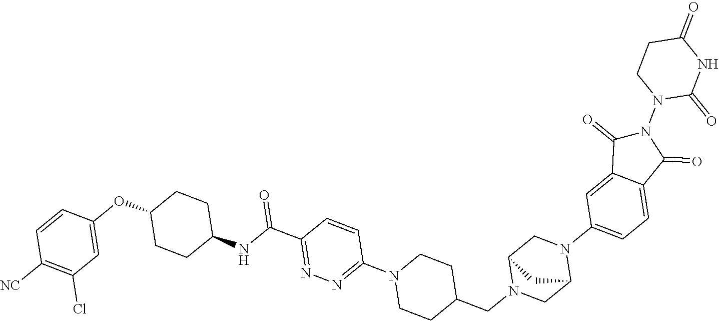 | N-((1r,4r)-4-(3-chloro-4- cyanophenoxy)cyclohexyl)-6-(4-(((1R,4R)-5- (2-(2,4-dioxotetrahydropyrimidin-1(2H)-yl)- 1,3-dioxoisoindolin-5-yl)-2,5- diazabicyclo[2.2.1]heptan-2- yl)methyl)piperidin-1-yl)pyridazine-3- carboxamide |
| GT- 0522 8 | 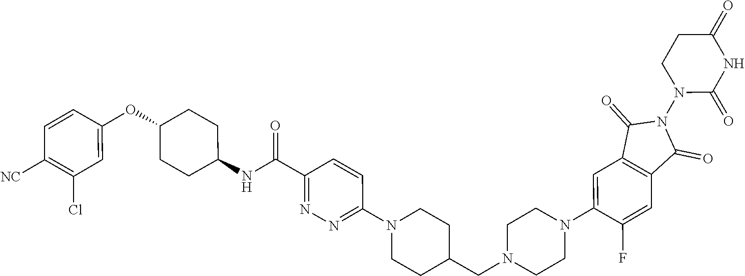 [SMILES: N#Cc1ccc(O[C@H]2CC[C@H](NC(=O)c3ccc(N4CCC(CN5CCN(c6cc7c(cc6F)C(=O)N(N6CCC(=O)NC6=O)C7=O)CC5)CC4)nn3)CC2)cc1Cl] | N-((1r,4r)-4-(3-chloro-4- cyanophenoxy)cyclohexyl)-6-(4-((4-(2-(2,4- dioxotetrahydropyrimidin-1(2H)-yl)-6-fluoro- 1,3-dioxoisoindolin-5-yl)piperazin-1- yl)methyl)piperidin-1-yl)pyridazine-3- carboxamide |
| GT- 0522 9 | 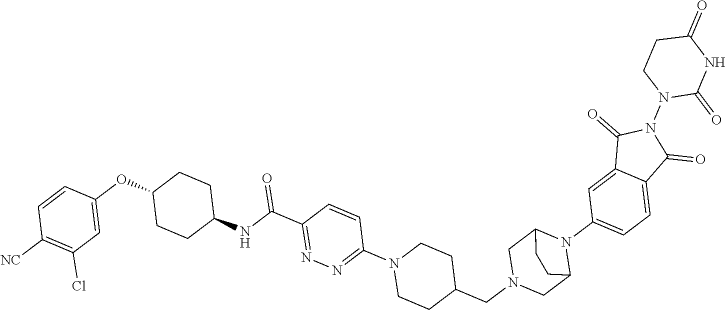 [SMILES: N#Cc1ccc(O[C@H]2CC[C@H](NC(=O)c3ccc(N4CCC(CN5CC6CCC(C5)N6c5ccc6c(c5)C(=O)N(N5CCC(=O)NC5=O)C6=O)CC4)nn3)CC2)cc1Cl] | N-((1r,4r)-4-(3-chloro-4- cyanophenoxy)cyclohexyl)-6-(4-((8-(2-(2,4- dioxotetrahydropyrimidin-1(2H)-yl)-1,3- dioxoisoindolin-5-yl)-3,8- diazabicyclo[3.2.1]octan-3- yl)methyl)piperidin-1-yl)pyridazine-3- carboxamide |
| GT- 0523 0 | 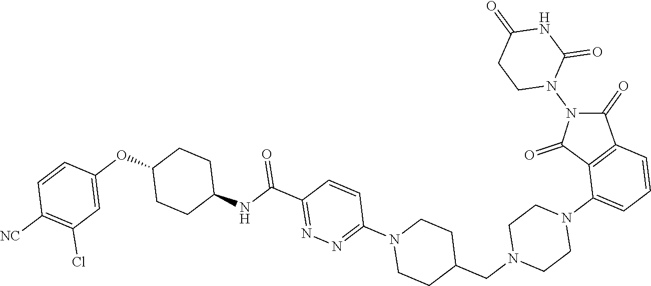 [SMILES: N#Cc1ccc(O[C@H]2CC[C@H](NC(=O)c3ccc(N4CCC(CN5CCN(c6cccc7c6C(=O)N(N6CCC(=O)NC6=O)C7=O)CC5)CC4)nn3)CC2)cc1Cl] | N-((1r,4r)-4-(3-chloro-4- cyanophenoxy)cyclohexyl)-6-(4-((4-(2-(2,4- dioxotetrahydropyrimidin-1(2H)-yl)-1,3- dioxoisoindolin-4-yl)piperazin-1- yl)methyl)piperidin-1-yl)pyridazine-3- carboxamide |
| GT- 0523 1 | 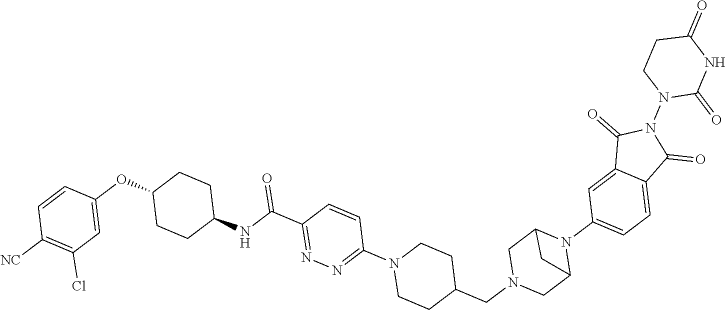 [SMILES: N#Cc1ccc(O[C@H]2CC[C@H](NC(=O)c3ccc(N4CCC(CN5CC6CC(C5)N6c5ccc6c(c5)C(=O)N(N5CCC(=O)NC5=O)C6=O)CC4)nn3)CC2)cc1Cl] | N-((1r,4r)-4-(3-chloro-4- cyanophenoxy)cyclohexyl)-6-(4-((6-(2-(2,4- dioxotetrahydropyrimidin-1(2H)-yl)-1,3- dioxoisoindolin-5-yl)-3,6- diazabicyclo[3.1.1]heptan-3- yl)methyl)piperidin-1-yl)pyridazine-3- carboxamide |
| GT- 0523 2 | 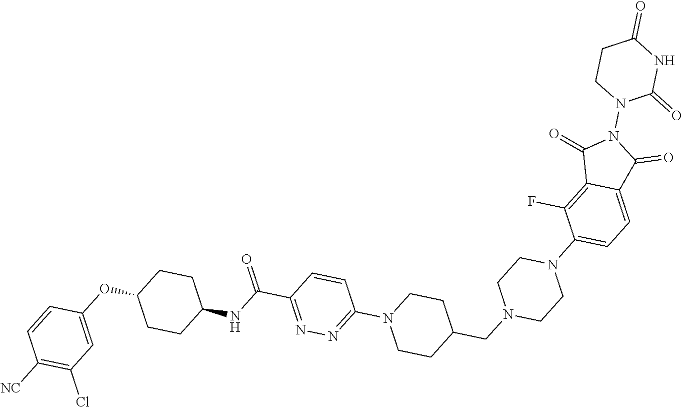 [SMILES: N#Cc1ccc(O[C@H]2CC[C@H](NC(=O)c3ccc(N4CCC(CN5CCN(c6ccc7c(c6F)C(=O)N(N6CCC(=O)NC6=O)C7=O)CC5)CC4)nn3)CC2)cc1Cl] | N-((1r,4r)-4-(3-chloro-4- cyanophenoxy)cyclohexyl)-6-(4-((4-(2-(2,4- dioxotetrahydropyrimidin-1(2H)-yl)-4-fluoro- 1,3-dioxoisoindolin-5-yl)piperazin-1- yl)methyl)piperidin-1-yl)pyridazine-3- carboxamide |
| GT- 0523 3 | 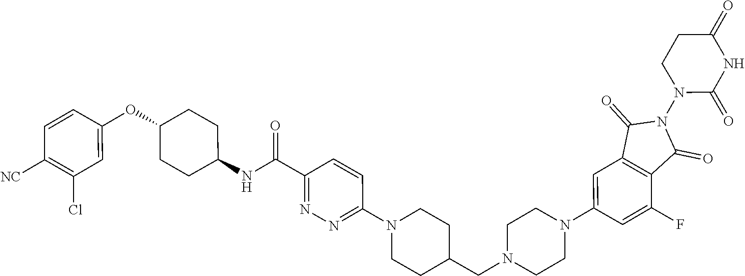 [SMILES: N#Cc1ccc(O[C@H]2CC[C@H](NC(=O)c3ccc(N4CCC(CN5CCN(c6cc(F)c7c(c6)C(=O)N(N6CCC(=O)NC6=O)C7=O)CC5)CC4)nn3)CC2)cc1Cl] | N-((1r,4r)-4-(3-chloro-4- cyanophenoxy)cyclohexyl)-6-(4-((4-(2-(2,4- dioxotetrahydropyrimidin-1(2H)-yl)-7-fluoro- 1,3-dioxoisoindolin-5-yl)piperazin-1- yl)methyl)piperidin-1-yl)pyridazine-3- carboxamide |
| GT- 0523 5 | 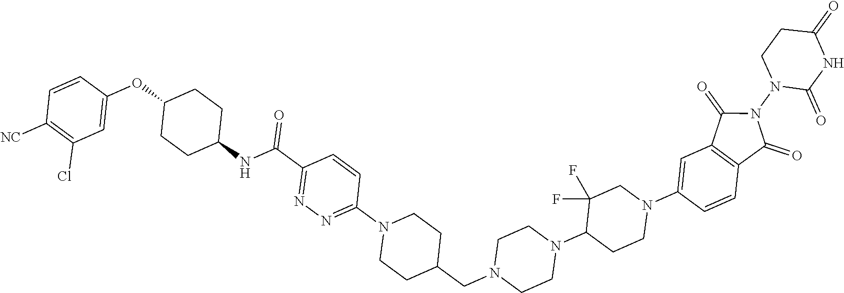 [SMILES: N#Cc1ccc(O[C@H]2CC[C@H](NC(=O)c3ccc(N4CCC(CN5CCN(C6CCN(c7ccc8c(c7)C(=O)N(N7CCC(=O)NC7=O)C8=O)CC6(F)F)CC5)CC4)nn3)CC2)cc1Cl] | N-((1r,4r)-4-(3-chloro-4- cyanophenoxy)cyclohexyl)-6-(4-((4-(1-(2- (2,4-dioxotetrahydropyrimidin-1(2H)-yl)-1,3- dioxoisoindolin-5-yl)-3,3-difluoropiperidin-4- yl)piperazin-1-yl)methyl)piperidin-1- yl)pyridazine-3-carboxamide |
| | 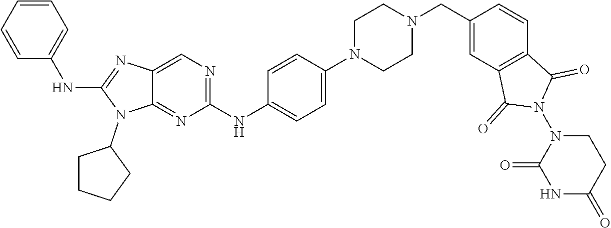 [SMILES: O=C1CCN(N2C(=O)c3ccc(CN4CCN(c5ccc(Nc6ncc7nc(Nc8ccccc8)n(C8CCCC8)c7n6)cc5)CC4)cc3C2=O)C(=O)N1] | 5-((4-(4-((9-cyclopentyl-8-(phenylamino)- 9H-purin-2-yl)amino)phenyl)piperazin-1- yl)methyl)-2-(2,4-dioxotetrahydropyrimidin- 1(2H)-yl)isoindoline-1,3-dione |
| GT- 0711 3 | 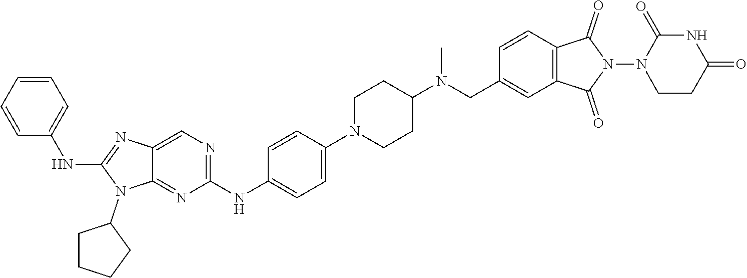 [SMILES: CN(Cc1ccc2c(c1)C(=O)N(N1CCC(=O)NC1=O)C2=O)C1CCN(c2ccc(Nc3ncc4nc(Nc5ccccc5)n(C5CCCC5)c4n3)cc2)CC1] | 5-(((1-(4-((9-cyclopentyl-8-(phenylamino)- 9H-purin-2-yl)amino)phenyl)piperidin-4- yl)(methyl)amino)methyl)-2-(2,4- dioxotetrahydropyrimidin-1(2H)- yl)isoindoline-1,3-dione |
| GT- 0612 4 | 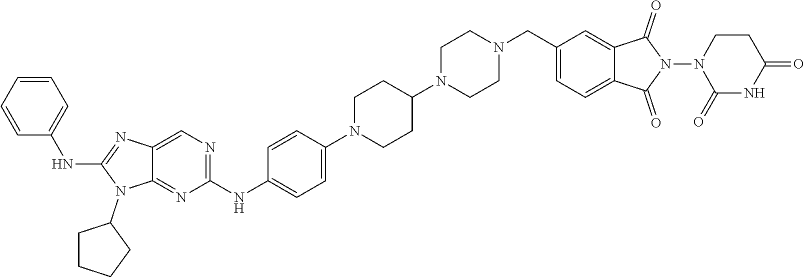 [SMILES: O=C1CCN(N2C(=O)c3ccc(CN4CCN(C5CCN(c6ccc(Nc7ncc8nc(Nc9ccccc9)n(C9CCCC9)c8n7)cc6)CC5)CC4)cc3C2=O)C(=O)N1] | 5-((4-(1-(4-((9-cyclopentyl-8-(phenylamino)- 9H-purin-2-yl)amino)phenyl)piperidin-4- yl)piperazin-1-yl)methyl)-2-(2,4- dioxotetrahydropyrimidin-1(2H)- yl)isoindoline-1,3-dione |
| | 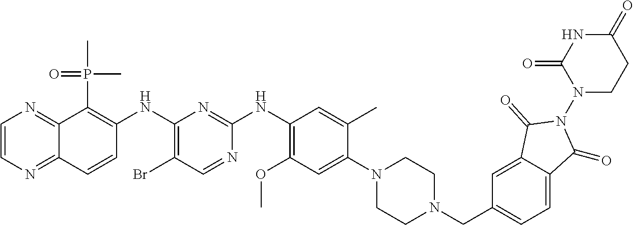 [SMILES: COc1cc(N2CCN(Cc3ccc4c(c3)C(=O)N(N3CCC(=O)NC3=O)C4=O)CC2)c(C)cc1Nc1ncc(Br)c(Nc2ccc3nccnc3c2P(C)(C)=O)n1] | 5-((4-(4-((5-bromo-4-((5- (dimethylphosphoryl)quinoxalin-6- yl)amino)pyrimidin-2-yl)amino)-5-methoxy- 2-methylphenyl)piperazin-1-yl)methyl)-2- (2,4-dioxotetrahydropyrimidin-1(2H)- yl)isoindoline-1,3-dione |
| GT- 0612 5 | 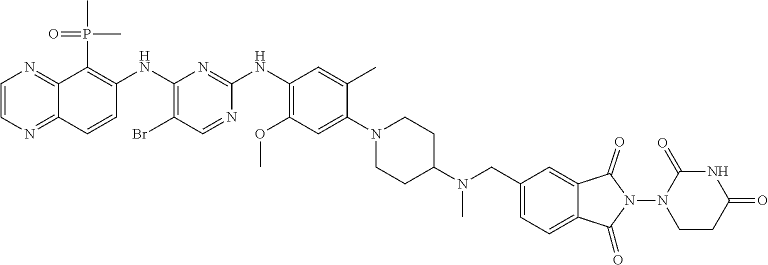 [SMILES: COc1cc(N2CCC(N(C)Cc3ccc4c(c3)C(=O)N(N3CCC(=O)NC3=O)C4=O)CC2)c(C)cc1Nc1ncc(Br)c(Nc2ccc3nccnc3c2P(C)(C)=O)n1] | 5-(((1-(4-((5-bromo-4-((5- (dimethylphosphoryl)quinoxalin-6- yl)amino)pyrimidin-2-yl)amino)-5-methoxy- 2-methylphenyl)piperidin-4- yl)(methyl)amino)methyl)-2-(2,4- dioxotetrahydropyrimidin-1(2H)- yl)isoindoline-1,3-dione |
| GT- 0615 4 | 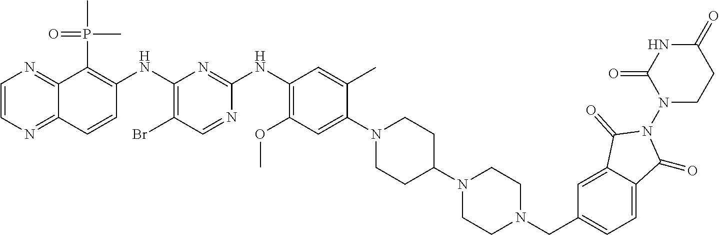 [SMILES: COc1cc(N2CCC(N3CCN(Cc4ccc5c(c4)C(=O)N(N4CCC(=O)NC4=O)C5=O)CC3)CC2)c(C)cc1Nc1ncc(Br)c(Nc2ccc3nccnc3c2P(C)(C)=O)n1] | 5-((4-(1-(4-((5-bromo-4-((5- (dimethylphosphoryl)quinoxalin-6- yl)amino)pyrimidin-2-yl)amino)-5-methoxy- 2-methylphenyl)piperidin-4-yl)piperazin-1- yl)methyl)-2-(2,4-dioxotetrahydropyrimidin- 1(2H)-yl)isoindoline-1,3-dione |
| | 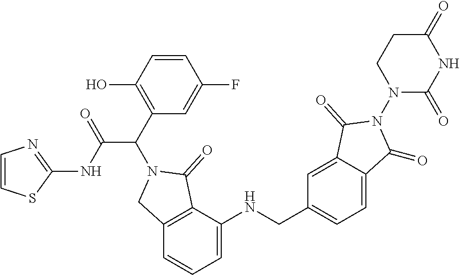 [SMILES: O=C1CCN(N2C(=O)c3ccc(CNc4cccc5c4C(=O)N(C(C(=O)Nc4nccs4)c4cc(F)ccc4O)C5)cc3C2=O)C(=O)N1] | 2-(7-(((2-(2,4-dioxotetrahydropyrimidin- 1(2H)-yl)-1,3-dioxoisoindolin-5- yl)methyl)amino)-1-oxoisoindolin-2-yl)-2-(5- fluoro-2-hydroxyphenyl)-N-(thiazol-2- yl)acetamide |
| | 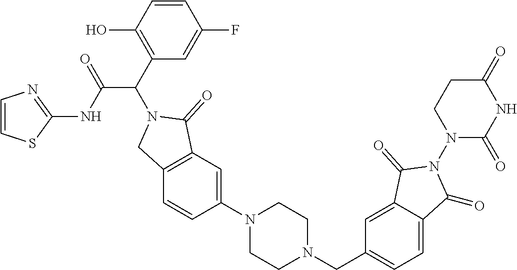 [SMILES: O=C1CCN(N2C(=O)c3ccc(CN4CCN(c5ccc6c(c5)C(=O)N(C(C(=O)Nc5nccs5)c5cc(F)ccc5O)C6)CC4)cc3C2=O)C(=O)N1] | 2-(6-(4-((2-(2,4-dioxotetrahydropyrimidin- 1(2H)-yl)-1,3-dioxoisoindolin-5- yl)methyl)piperazin-1-yl)-1-oxoisoindolin-2- yl)-2-(5-fluoro-2-hydroxyphenyl)-N-(thiazol- 2-yl)acetamide |
| | 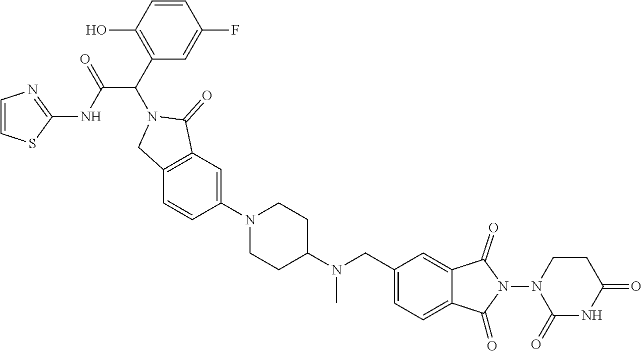 [SMILES: CN(Cc1ccc2c(c1)C(=O)N(N1CCC(=O)NC1=O)C2=O)C1CCN(c2ccc3c(c2)C(=O)N(C(C(=O)Nc2nccs2)c2cc(F)ccc2O)C3)CC1] | 2-(6-(4-(((2-(2,4-dioxotetrahydropyrimidin- 1(2H)-yl)-1,3-dioxoisoindolin-5- yl)methyl)(methyl)amino)piperidin-1-yl)-1- oxoisoindolin-2-yl)-2-(5-fluoro-2- hydroxyphenyl)-N-(thiazol-2-yl)acetamide |
| | 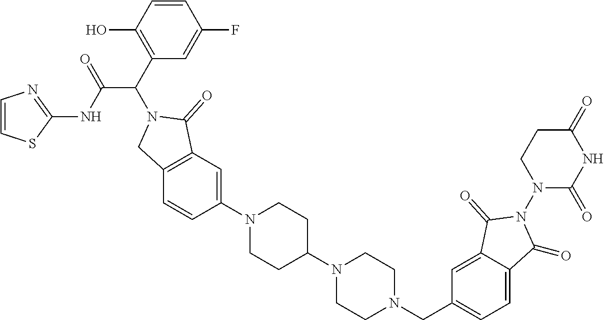 [SMILES: O=C1CCN(N2C(=O)c3ccc(CN4CCN(C5CCN(c6ccc7c(c6)C(=O)N(C(C(=O)Nc6nccs6)c6cc(F)ccc6O)C7)CC5)CC4)cc3C2=O)C(=O)N1] | 2-(6-(4-(4-((2-(2,4-dioxotetrahydropyrimidin- 1(2H)-yl)-1,3-dioxoisoindolin-5- yl)methyl)piperazin-1-yl)piperidin-1-yl)-1- oxoisoindolin-2-yl)-2-(5-fluoro-2- hydroxyphenyl)-N-(thiazol-2-yl)acetamide |
| | 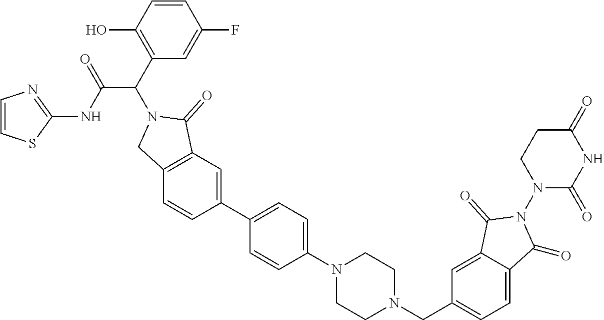 [SMILES: O=C1CCN(N2C(=O)c3ccc(CN4CCN(c5ccc(-c6ccc7c(c6)C(=O)N(C(C(=O)Nc6nccs6)c6cc(F)ccc6O)C7)cc5)CC4)cc3C2=O)C(=O)N1] | 2-(6-(4-(4-((2-(2,4-dioxotetrahydropyrimidin- 1(2H)-yl)-1,3-dioxoisoindolin-5- yl)methyl)piperazin-1-yl)phenyl)-1- oxoisoindolin-2-yl)-2-(5-fluoro-2- hydroxyphenyl)-N-(thiazol-2-yl)acetamide |
| | 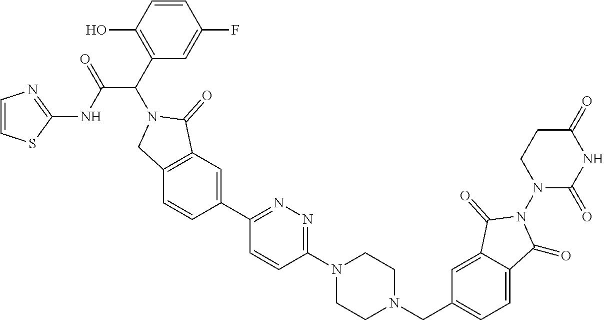 [SMILES: O=C1CCN(N2C(=O)c3ccc(CN4CCN(c5ccc(-c6ccc7c(c6)C(=O)N(C(C(=O)Nc6nccs6)c6cc(F)ccc6O)C7)nn5)CC4)cc3C2=O)C(=O)N1] | 2-(6-(6-(4-((2-(2,4-dioxotetrahydropyrimidin- 1(2H)-yl)-1,3-dioxoisoindolin-5- yl)methyl)piperazin-1-yl)pyridazin-3-yl)-1- oxoisoindolin-2-yl)-2-(5-fluoro-2- hydroxyphenyl)-N-(thiazol-2-yl)acetamide |
| GT- 0616 0 | 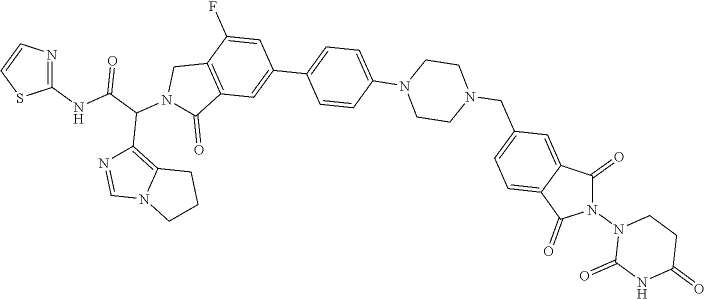 | 2-(6,7-dihydro-5H-pyrrolo[1,2-c]imidazol-1- yl)-2-(6-(4-(4-((2-(2,4- dioxotetrahydropyrimidin-1(2H)-yl)-1,3- dioxoisoindolin-5-yl)methyl)piperazin-1- yl)phenyl)-4-fluoro-1-oxoisoindolin-2-yl)-N- (thiazol-2-yl)acetamide |
| | 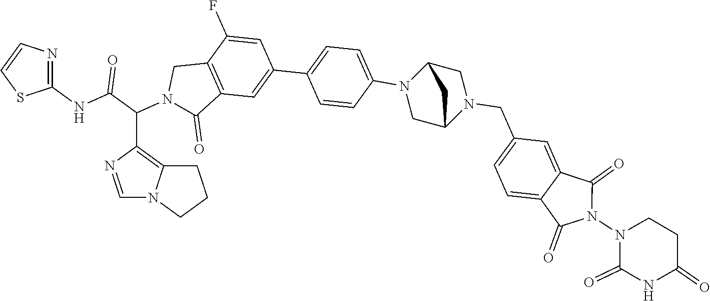 [SMILES: O=C1CCN(N2C(=O)c3ccc(CN4C[C@@H]5C[C@H]4CN5c4ccc(-c5cc(F)c6c(c5)C(=O)N(C(C(=O)Nc5nccs5)c5ncn7c5CCC7)C6)cc4)cc3C2=O)C(=O)N1] | 2-(6,7-dihydro-5H-pyrrolo[1,2-c]imidazol-1- yl)-2-(6-(4-((1S,4S)-5-((2-(2,4- dioxotetrahydropyrimidin-1(2H)-yl)-1,3- dioxoisoindolin-5-yl)methyl)-2,5- diazabicyclo[2.2.1]heptan-2-yl)phenyl)-4- fluoro-1-oxoisoindolin-2-yl)-N-(thiazol-2- yl)acetamide |
| GT- 0617 3 | 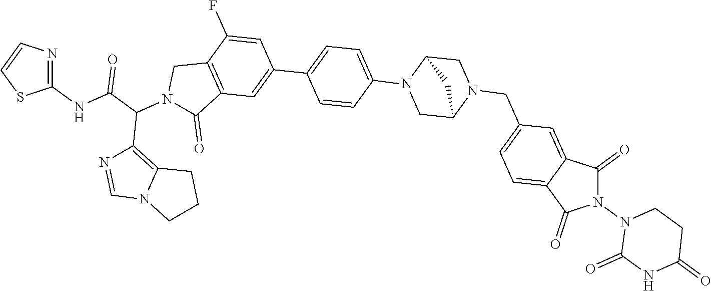 [SMILES: O=C1CCN(N2C(=O)c3ccc(CN4C[C@H]5C[C@@H]4CN5c4ccc(-c5cc(F)c6c(c5)C(=O)N(C(C(=O)Nc5nccs5)c5ncn7c5CCC7)C6)cc4)cc3C2=O)C(=O)N1] | 2-(6,7-dihydro-5H-pyrrolo[1,2-c]imidazol-1- yl)-2-(6-(4-((1R,4R)-5-((2-(2,4- dioxotetrahydropyrimidin-1(2H)-yl)-1,3- dioxoisoindolin-5-yl)methyl)-2,5- diazabicyclo[2.2.1]heptan-2-yl)phenyl)-4- fluoro-1-oxoisoindolin-2-yl)-N-(thiazol-2- yl)acetamide |
| GT- 0625 1 | 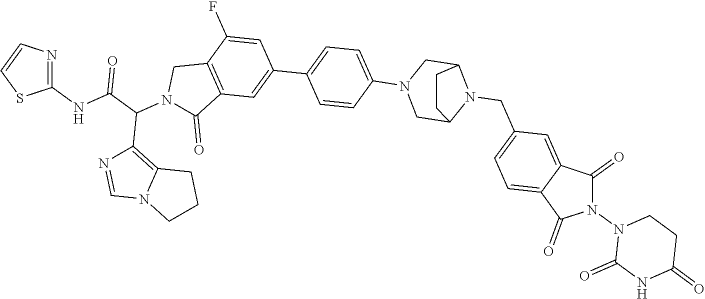 | 2-(6,7-dihydro-5H-pyrrolo[1,2-c]imidazol-1- yl)-2-(6-(4-(8-((2-(2,4- dioxotetrahydropyrimidin-1(2H)-yl)-1,3- dioxoisoindolin-5-yl)methyl)-3,8- diazabicyclo[3.2.1]octan-3-yl)phenyl)-4- fluoro-1-oxoisoindolin-2-yl)-N-(thiazol-2- yl)acetamide |
| GT- 0519 6 | 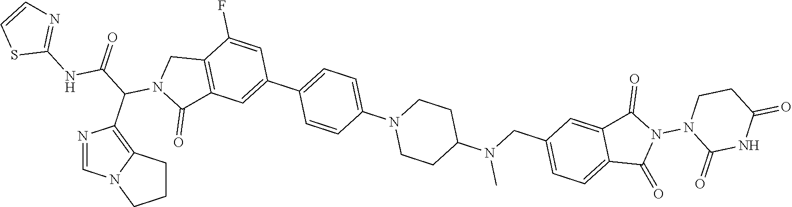 [SMILES: CN(Cc1ccc2c(c1)C(=O)N(N1CCC(=O)NC1=O)C2=O)C1CCN(c2ccc(-c3cc(F)c4c(c3)C(=O)N(C(C(=O)Nc3nccs3)c3ncn5c3CCC5)C4)cc2)CC1] | 2-(6,7-dihydro-5H-pyrrolo[1,2-c]imidazol-1- yl)-2-(6-(4-(4-(((2-(2,4- dioxotetrahydropyrimidin-1(2H)-yl)-1,3- dioxoisoindolin-5- yl)methyl)(methyl)amino)piperidin-1- yl)phenyl)-4-fluoro-1-oxoisoindolin-2-yl)-N- (thiazol-2-yl)acetamide |
| GT- 0624 2 | 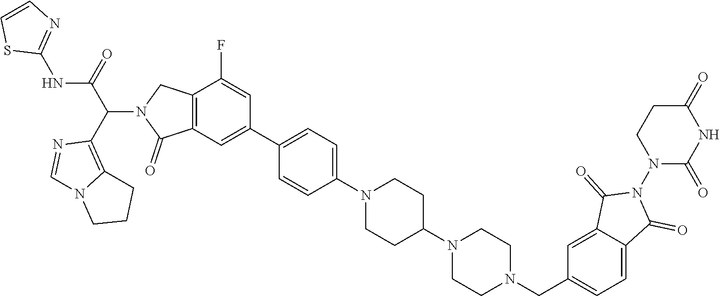 [SMILES: O=C1CCN(N2C(=O)c3ccc(CN4CCN(C5CCN(c6ccc(-c7cc(F)c8c(c7)C(=O)N(C(C(=O)Nc7nccs7)c7ncn9c7CCC9)C8)cc6)CC5)CC4)cc3C2=O)C(=O)N1] | 2-(6,7-dihydro-5H-pyrrolo[1,2-c]imidazol-1- yl)-2-(6-(4-(4-(4-((2-(2,4- dioxotetrahydropyrimidin-1(2H)-yl)-1,3- dioxoisoindolin-5-yl)methyl)piperazin-1- yl)piperidin-1-yl)phenyl)-4-fluoro-1- oxoisoindolin-2-yl)-N-(thiazol-2- yl)acetamide |
| GT- 0631 6 | 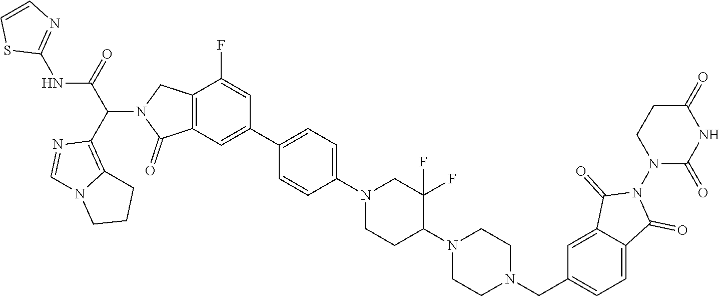 [SMILES: O=C1CCN(N2C(=O)c3ccc(CN4CCN(C5CCN(c6ccc(-c7cc(F)c8c(c7)C(=O)N(C(C(=O)Nc7nccs7)c7ncn9c7CCC9)C8)cc6)CC5(F)F)CC4)cc3C2=O)C(=O)N1] | 2-(6,7-dihydro-5H-pyrrolo[1,2-c]imidazol-1- yl)-2-(6-(4-(4-(4-((2-(2,4- dioxotetrahydropyrimidin-1(2H)-yl)-1,3- dioxoisoindolin-5-yl)methyl)piperazin-1-yl)- 3,3-difluoropiperidin-1-yl)phenyl)-4-fluoro-1- oxoisoindolin-2-yl)-N-(thiazol-2- yl)acetamide |
| GT- 0632 5 | 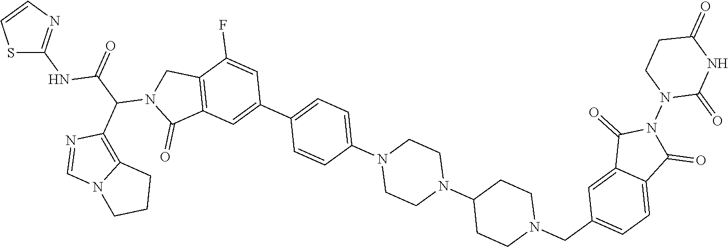 [SMILES: O=C1CCN(N2C(=O)c3ccc(CN4CCC(N5CCN(c6ccc(-c7cc(F)c8c(c7)C(=O)N(C(C(=O)Nc7nccs7)c7ncn9c7CCC9)C8)cc6)CC5)CC4)cc3C2=O)C(=O)N1] | 2-(6,7-dihydro-5H-pyrrolo[1,2-c]imidazol-1- yl)-2-(6-(4-(4-(1-((2-(2,4- dioxotetrahydropyrimidin-1(2H)-yl)-1,3- dioxoisoindolin-5-yl)methyl)piperidin-4- yl)piperazin-1-yl)phenyl)-4-fluoro-1- oxoisoindolin-2-yl)-N-(thiazol-2- yl)acetamide |
| | 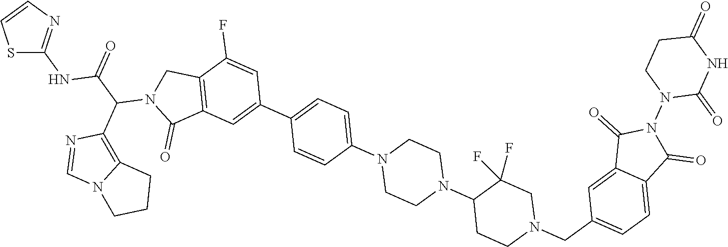 [SMILES: O=C1CCN(N2C(=O)c3ccc(CN4CCC(N5CCN(c6ccc(-c7cc(F)c8c(c7)C(=O)N(C(C(=O)Nc7nccs7)c7ncn9c7CCC9)C8)cc6)CC5)C(F)(F)C4)cc3C2=O)C(=O)N1] | 2-(6,7-dihydro-5H-pyrrolo[1,2-c]imidazol-1- yl)-2-(6-(4-(4-(1-((2-(2,4- dioxotetrahydropyrimidin-1(2H)-yl)-1,3- dioxoisoindolin-5-yl)methyl)-3,3- difluoropiperidin-4-yl)piperazin-1-yl)phenyl)- 4-fluoro-1-oxoisoindolin-2-yl)-N-(thiazol-2- yl)acetamide |
| | 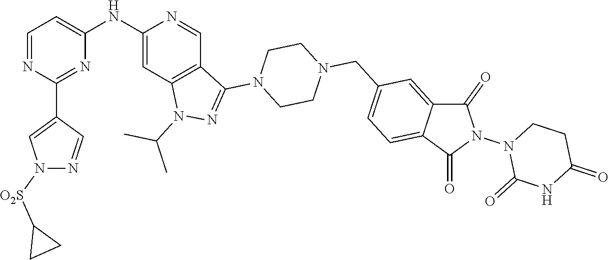 [SMILES: CC(C)n1nc(N2CCN(Cc3ccc4c(c3)C(=O)N(N3CCC(=O)NC3=O)C4=O)CC2)c2cnc(Nc3ccnc(-c4cnn(S(=O)(=O)C5CC5)c4)n3)cc21] | 5-((4-(6-((2-(1-(cyclopropylsulfonyl)-1H- pyrazol-4-yl)pyrimidin-4-yl)amino)-1- isopropyl-1H-pyrazolo[4,3-c]pyridin-3- yl)piperazin-1-yl)methyl)-2-(2,4- dioxotetrahydropyrimidin-1(2H)- yl)isoindoline-1,3-dione |
| | 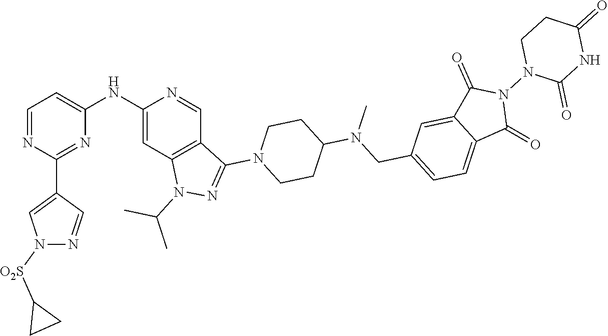 [SMILES: CC(C)n1nc(N2CCC(N(C)Cc3ccc4c(c3)C(=O)N(N3CCC(=O)NC3=O)C4=O)CC2)c2cnc(Nc3ccnc(-c4cnn(S(=O)(=O)C5CC5)c4)n3)cc21] | 5-(((1-(6-((2-(1-(cyclopropylsulfonyl)-1H- pyrazol-4-yl)pyrimidin-4-yl)amino)-1- isopropyl-1H-pyrazolo[4,3-c]pyridin-3- yl)piperidin-4-yl)(methyl)amino)methyl)-2- (2,4-dioxotetrahydropyrimidin-1(2H)- yl)isoindoline-1,3-dione |
| GT- 0612 6 | 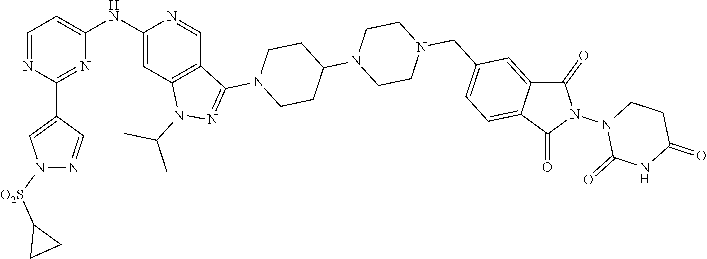 [SMILES: CC(C)n1nc(N2CCC(N3CCN(Cc4ccc5c(c4)C(=O)N(N4CCC(=O)NC4=O)C5=O)CC3)CC2)c2cnc(Nc3ccnc(-c4cnn(S(=O)(=O)C5CC5)c4)n3)cc21] | 5-((4-(1-(6-((2-(1-(cyclopropylsulfonyl)-1H- pyrazol-4-yl)pyrimidin-4-yl)amino)-1- isopropyl-1H-pyrazolo[4,3-c]pyridin-3- yl)piperidin-4-yl)piperazin-1-yl)methyl)-2- (2,4-dioxotetrahydropyrimidin-1(2H)- yl)isoindoline-1,3-dione |
| GT- 0612 7 | 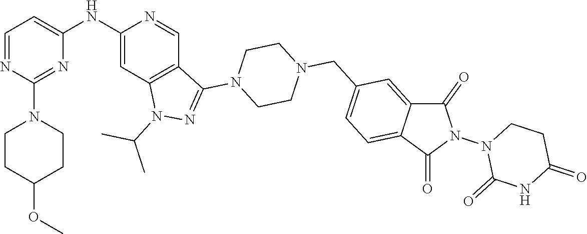 [SMILES: COC1CCN(c2nccc(Nc3cc4c(cn3)c(N3CCN(Cc5ccc6c(c5)C(=O)N(N5CCC(=O)NC5=O)C6=O)CC3)nn4C(C)C)n2)CC1] | 2-(2,4-dioxotetrahydropyrimidin-1(2H)-yl)-5- ((4-(1-isopropyl-6-((2-(4-methoxypiperidin- 1-yl)pyrimidin-4-yl)amino)-1H-pyrazolo[4,3- c]pyridin-3-yl)piperazin-1- yl)methyl)isoindoline-1,3-dione |
| GT- 0611 0 | 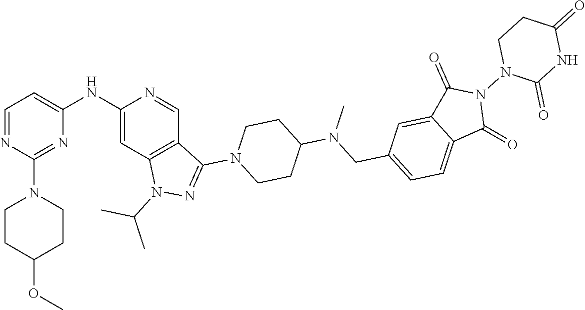 [SMILES: COC1CCN(c2nccc(Nc3cc4c(cn3)c(N3CCC(N(C)Cc5ccc6c(c5)C(=O)N(N5CCC(=O)NC5=O)C6=O)CC3)nn4C(C)C)n2)CC1] | 2-(2,4-dioxotetrahydropyrimidin-1(2H)-yl)-5- (((1-(1-isopropyl-6-((2-(4-methoxypiperidin- 1-yl)pyrimidin-4-yl)amino)-1H-pyrazolo[4,3- c]pyridin-3-yl)piperidin-4- yl)(methyl)amino)methyl)isoindoline-1,3- dione |
| | 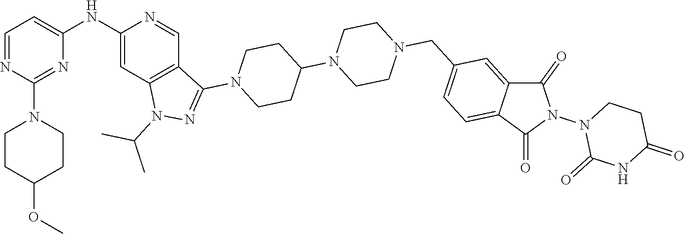 [SMILES: COC1CCN(c2nccc(Nc3cc4c(cn3)c(N3CCC(N5CCN(Cc6ccc7c(c6)C(=O)N(N6CCC(=O)NC6=O)C7=O)CC5)CC3)nn4C(C)C)n2)CC1] | 2-(2,4-dioxotetrahydropyrimidin-1(2H)-yl)-5- ((4-(1-(1-isopropyl-6-((2-(4- methoxypiperidin-1-yl)pyrimidin-4- yl)amino)-1H-pyrazolo[4,3-c]pyridin-3- yl)piperidin-4-yl)piperazin-1- yl)methyl)isoindoline-1,3-dione |
| | 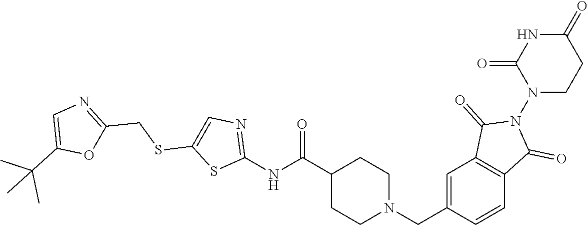 [SMILES: CC(C)(C)c1cnc(CSc2cnc(NC(=O)C3CCN(Cc4ccc5c(c4)C(=O)N(N4CCC(=O)NC4=O)C5=O)CC3)s2)o1] | N-(5-(((5-(tert-butyl)oxazol-2- yl)methyl)thio)thiazol-2-yl)-1-((2-(2,4- dioxotetrahydropyrimidin-1(2H)-yl)-1,3- dioxoisoindolin-5-yl)methyl)piperidine-4- carboxamide |
| | 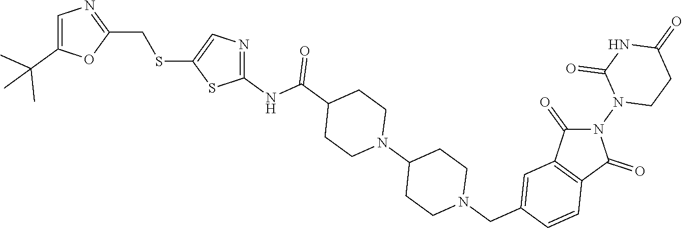 [SMILES: CC(C)(C)c1cnc(CSc2cnc(NC(=O)C3CCN(C4CCN(Cc5ccc6c(c5)C(=O)N(N5CCC(=O)NC5=O)C6=O)CC4)CC3)s2)o1] | N-(5-(((5-(tert-butyl)oxazol-2- yl)methyl)thio)thiazol-2-yl)-1′-((2-(2,4- dioxotetrahydropyrimidin-1(2H)-yl)-1,3- dioxoisoindolin-5-yl)methyl)-[1,4′- bipiperidine]-4-carboxamide |
| | 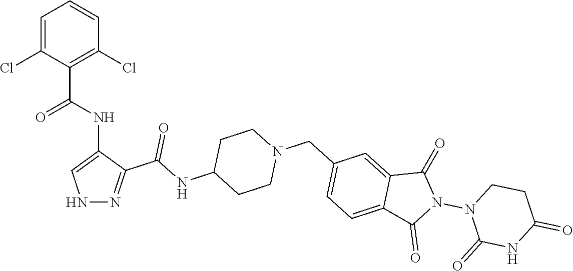 [SMILES: O=C1CCN(N2C(=O)c3ccc(CN4CCC(NC(=O)c5n[nH]cc5NC(=O)c5c(Cl)cccc5Cl)CC4)cc3C2=O)C(=O)N1] | 4-(2,6-dichlorobenzamido)-N-(1-((2-(2,4- dioxotetrahydropyrimidin-1(2H)-yl)-1,3- dioxoisoindolin-5-yl)methyl)piperidin-4-yl)- 1H-pyrazole-3-carboxamide |
| | 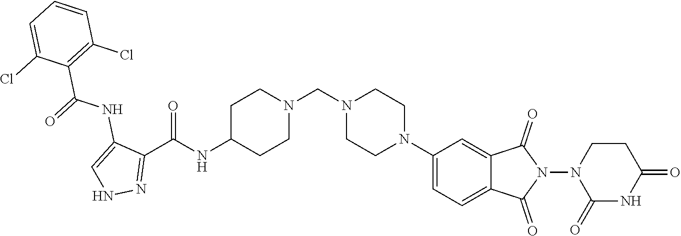 [SMILES: O=C1CCN(N2C(=O)c3ccc(N4CCN(CN5CCC(NC(=O)c6n[nH]cc6NC(=O)c6c(Cl)cccc6Cl)CC5)CC4)cc3C2=O)C(=O)N1] | 4-(2,6-dichlorobenzamido)-N-(1-((4-(2-(2,4- dioxotetrahydropyrimidin-1(2H)-yl)-1,3- dioxoisoindolin-5-yl)piperazin-1- yl)methyl)piperidin-4-yl)-1H-pyrazole-3- carboxamide |
| | 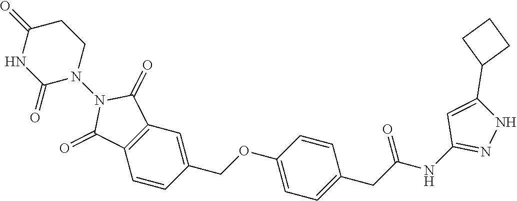 [SMILES: O=C1CCN(N2C(=O)c3ccc(COc4ccc(CC(=O)Nc5cc(C6CCC6)[nH]n5)cc4)cc3C2=O)C(=O)N1] | N-(5-cyclobutyl-1H-pyrazol-3-yl)-2-(4-((2- (2,4-dioxotetrahydropyrimidin-1(2H)-yl)-1,3- dioxoisoindolin-5- yl)methoxy)phenyl)acetamide |
| GT- 0821 3 | 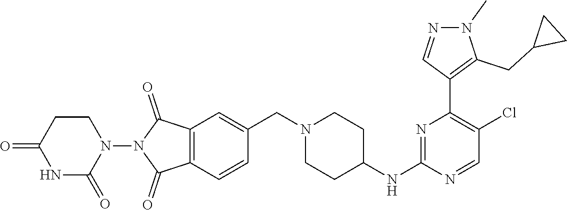 [SMILES: Cn1ncc(-c2nc(NC3CCN(Cc4ccc5c(c4)C(=O)N(N4CCC(=O)NC4=O)C5=O)CC3)ncc2Cl)c1CC1CC1] | 5-((4-((5-chloro-4-(5-(cyclopropylmethyl)-1- methyl-1H-pyrazol-4-yl)pyrimidin-2- yl)amino)piperidin-1-yl)methyl)-2-(2,4- dioxotetrahydropyrimidin-1(2H)- yl)isoindoline-1,3-dione |
| GT- 0815 4 | 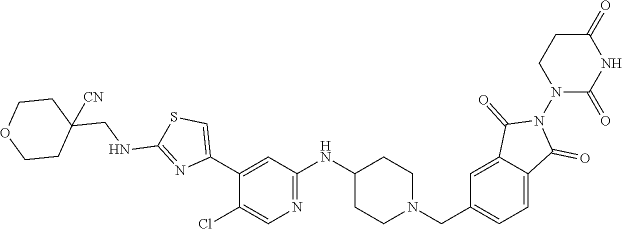 [SMILES: N#CC1(CNc2nc(-c3cc(NC4CCN(Cc5ccc6c(c5)C(=O)N(N5CCC(=O)NC5=O)C6=O)CC4)ncc3Cl)cs2)CCOCC1] | 4-(((4-(5-chloro-2-((1-((2-(2,4- dioxotetrahydropyrimidin-1(2H)-yl)-1,3- dioxoisoindolin-5-yl)methyl)piperidin-4- yl)amino)pyridin-4-yl)thiazol-2- yl)amino)methyl)tetrahydro-2H-pyran-4- carbonitrile |
| GT- 0821 5 | 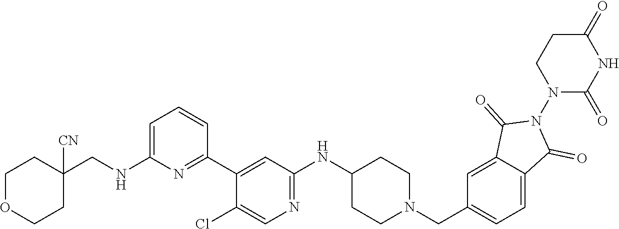 [SMILES: N#CC1(CNc2cccc(-c3cc(NC4CCN(Cc5ccc6c(c5)C(=O)N(N5CCC(=O)NC5=O)C6=O)CC4)ncc3Cl)n2)CCOCC1] | 4-(((5′-chloro-2′-((1-((2-(2,4- dioxotetrahydropyrimidin-1(2H)-yl)-1,3- dioxoisoindolin-5-yl)methyl)piperidin-4- yl)amino)-[2,4′-bipyridin]-6- yl)amino)methyl)tetrahydro-2H-pyran-4- carbonitrile |
| GT- 0822 3 | 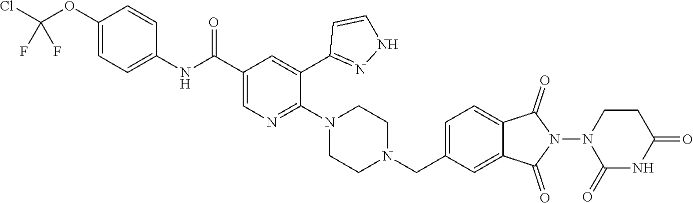 [SMILES: O=C1CCN(N2C(=O)c3ccc(CN4CCN(c5ncc(C(=O)Nc6ccc(OC(F)(F)Cl)cc6)cc5-c5cc[nH]n5)CC4)cc3C2=O)C(=O)N1] | N-(4-(chlorodifluoromethoxy)phenyl)-6-(4- ((2-(2,4-dioxotetrahydropyrimidin-1(2H)-yl)- 1,3-dioxoisoindolin-5-yl)methyl)piperazin-1- yl)-5-(1H-pyrazol-3-yl)nicotinamide |
| | 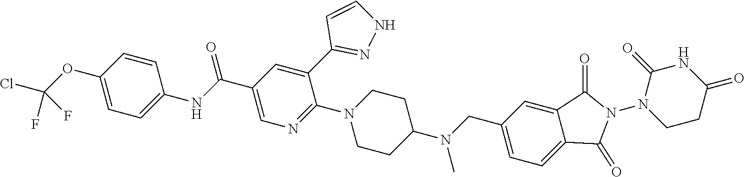 [SMILES: CN(Cc1ccc2c(c1)C(=O)N(N1CCC(=O)NC1=O)C2=O)C1CCN(c2ncc(C(=O)Nc3ccc(OC(F)(F)Cl)cc3)cc2-c2cc[nH]n2)CC1] | N-(4-(chlorodifluoromethoxy)phenyl)-6-(4- (((2-(2,4-dioxotetrahydropyrimidin-1(2H)- yl)-1,3-dioxoisoindolin-5- yl)methyl)(methyl)amino)piperidin-1-yl)-5- (1H-pyrazol-3-yl)nicotinamide |
| GT- 0822 5 | 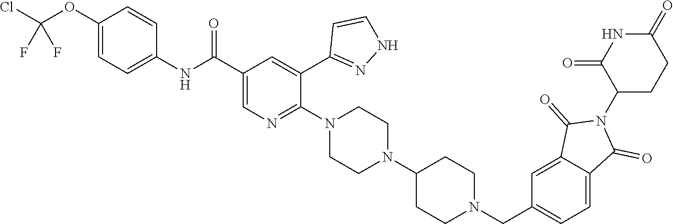 [SMILES: O=C1CCC(N2C(=O)c3ccc(CN4CCC(N5CCN(c6ncc(C(=O)Nc7ccc(OC(F)(F)Cl)cc7)cc6-c6cc[nH]n6)CC5)CC4)cc3C2=O)C(=O)N1] | N-(4-(chlorodifluoromethoxy)phenyl)-6-(4- (1-((2-(2,4-dioxotetrahydropyrimidin-1(2H)- yl)-1,3-dioxoisoindolin-5- yl)methyl)piperidin-4-yl)piperazin-1-yl)-5- (1H-pyrazol-3-yl)nicotinamide |
| GT- 0822 7 | 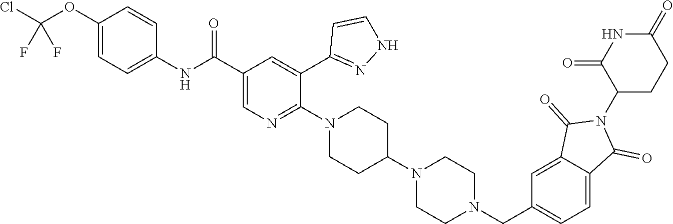 [SMILES: O=C1CCC(N2C(=O)c3ccc(CN4CCN(C5CCN(c6ncc(C(=O)Nc7ccc(OC(F)(F)Cl)cc7)cc6-c6cc[nH]n6)CC5)CC4)cc3C2=O)C(=O)N1] | N-(4-(chlorodifluoromethoxy)phenyl)-6-(4- (4-((2-(2,4-dioxotetrahydropyrimidin-1(2H)- yl)-1,3-dioxoisoindolin-5- yl)methyl)piperazin-1-yl)piperidin-1-yl)-5- (1H-pyrazol-3-yl)nicotinamide |
| GT- 0821 7 | 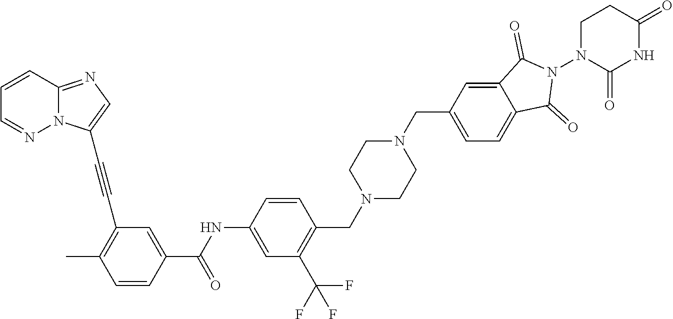 [SMILES: Cc1ccc(C(=O)Nc2ccc(CN3CCN(Cc4ccc5c(c4)C(=O)N(N4CCC(=O)NC4=O)C5=O)CC3)c(C(F)(F)F)c2)cc1C#Cc1cnc2cccnn12] | N-(4-((4-((2-(2,4-dioxotetrahydropyrimidin- 1(2H)-yl)-1,3-dioxoisoindolin-5- yl)methyl)piperazin-1-yl)methyl)-3- (trifluoromethyl)phenyl)-3-(imidazo[1,2- b]pyridazin-3-ylethynyl)-4-methylbenzamide |
| GT- 0821 9 | 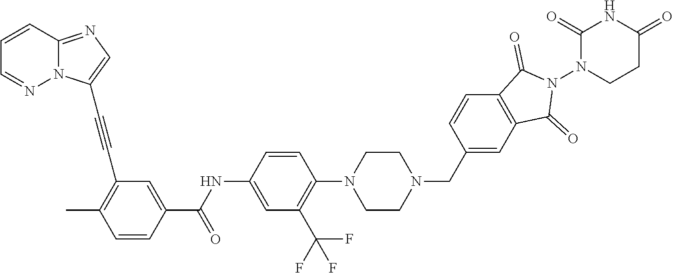 [SMILES: Cc1ccc(C(=O)Nc2ccc(N3CCN(Cc4ccc5c(c4)C(=O)N(N4CCC(=O)NC4=O)C5=O)CC3)c(C(F)(F)F)c2)cc1C#Cc1cnc2cccnn12] | N-(4-(4-((2-(2,4-dioxotetrahydropyrimidin- 1(2H)-yl)-1,3-dioxoisoindolin-5- yl)methyl)piperazin-1-yl)-3- (trifluoromethyl)phenyl)-3-(imidazo[1,2- b]pyridazin-3-ylethynyl)-4-methylbenzamide |
| | 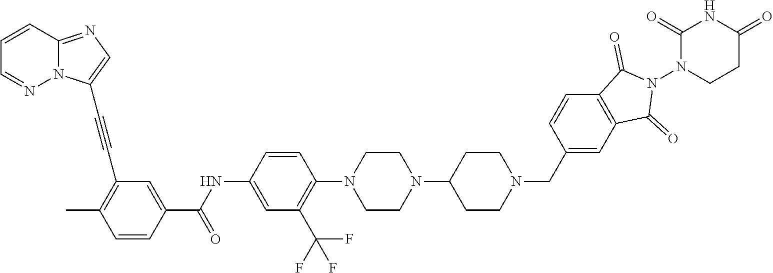 [SMILES: Cc1ccc(C(=O)Nc2ccc(N3CCN(C4CCN(Cc5ccc6c(c5)C(=O)N(N5CCC(=O)NC5=O)C6=O)CC4)CC3)c(C(F)(F)F)c2)cc1C#Cc1cnc2cccnn12] | N-(4-(4-(1-((2-(2,4- dioxotetrahydropyrimidin-1(2H)-yl)-1,3- dioxoisoindolin-5-yl)methyl)piperidin-4- yl)piperazin-1-yl)-3-(trifluoromethyl)phenyl)- 3-(imidazo[1,2-b]pyridazin-3-ylethynyl)-4- methylbenzamide |
| GT- 0822 1 | 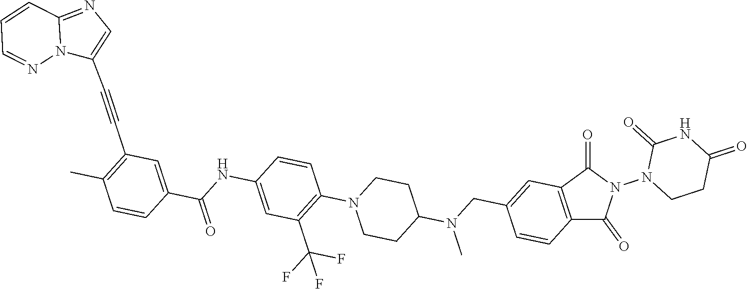 [SMILES: Cc1ccc(C(=O)Nc2ccc(N3CCC(N(C)Cc4ccc5c(c4)C(=O)N(N4CCC(=O)NC4=O)C5=O)CC3)c(C(F)(F)F)c2)cc1C#Cc1cnc2cccnn12] | N-(4-(4-(((2-(2,4-dioxotetrahydropyrimidin- 1(2H)-yl)-1,3-dioxoisoindolin-5- yl)methyl)(methyl)amino)piperidin-1-yl)-3- (trifluoromethyl)phenyl)-3-(imidazo[1,2- b]pyridazin-3-ylethynyl)-4-methylbenzamide |
| GT- 0633 6 | 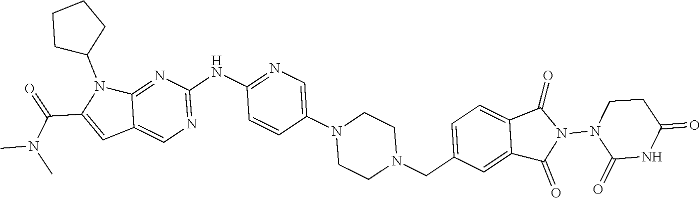 [SMILES: CN(C)C(=O)c1cc2cnc(Nc3ccc(N4CCN(Cc5ccc6c(c5)C(=O)N(N5CCC(=O)NC5=O)C6=O)CC4)cn3)nc2n1C1CCCC1] | 7-cyclopentyl-2-((5-(4-((2-(2,4- dioxotetrahydropyrimidin-1(2H)-yl)-1,3- dioxoisoindolin-5-yl)methyl)piperazin-1- yl)pyridin-2-yl)amino)-N,N-dimethyl-7H- pyrrolo[2,3-d]pyrimidine-6-carboxamide |
| GT- 0512 1 | 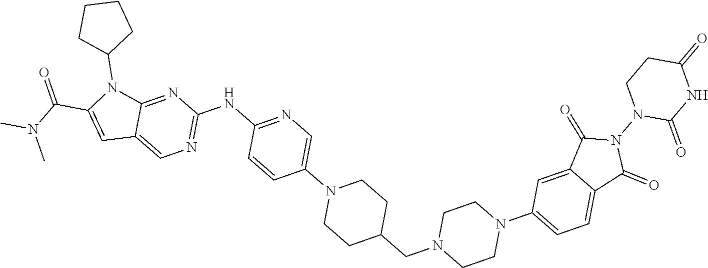 [SMILES: CN(C)C(=O)c1cc2cnc(Nc3ccc(N4CCC(CN5CCN(c6ccc7c(c6)C(=O)N(N6CCC(=O)NC6=O)C7=O)CC5)CC4)cn3)nc2n1C1CCCC1] | 7-cyclopentyl-2-((5-(4-((4-(2-(2,4- dioxotetrahydropyrimidin-1(2H)-yl)-1,3- dioxoisoindolin-5-yl)piperazin-1- yl)methyl)piperidin-1-yl)pyridin-2-yl)amino)- N,N-dimethyl-7H-pyrrolo[2,3-d]pyrimidine- 6-carboxamide |
| GT- 0648 8 | 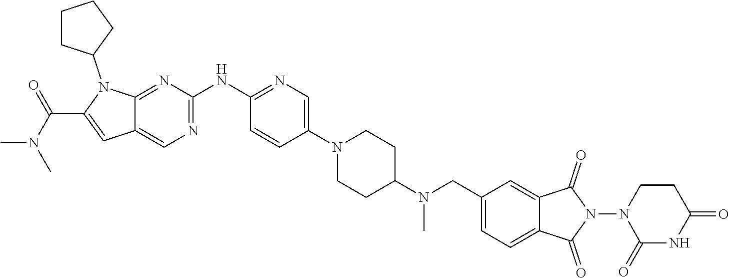 [SMILES: CN(C)C(=O)c1cc2cnc(Nc3ccc(N4CCC(N(C)Cc5ccc6c(c5)C(=O)N(N5CCC(=O)NC5=O)C6=O)CC4)cn3)nc2n1C1CCCC1] | 7-cyclopentyl-2-((5-(4-(((2-(2,4- dioxotetrahydropyrimidin-1(2H)-yl)-1,3- dioxoisoindolin-5- yl)methyl)(methyl)amino)piperidin-1- yl)pyridin-2-yl)amino)-N,N-dimethyl-7H- pyrrolo[2,3-d]pyrimidine-6-carboxamide |
| GT- 0649 8 | 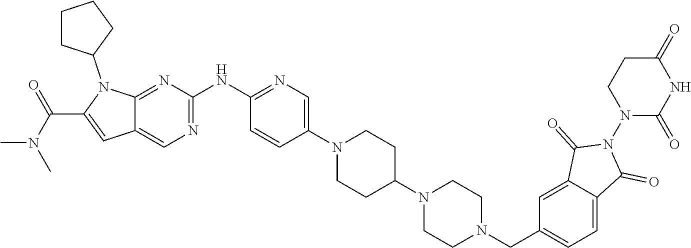 [SMILES: CN(C)C(=O)c1cc2cnc(Nc3ccc(N4CCC(N5CCN(Cc6ccc7c(c6)C(=O)N(N6CCC(=O)NC6=O)C7=O)CC5)CC4)cn3)nc2n1C1CCCC1] | 7-cyclopentyl-2-((5-(4-(4-((2-(2,4- dioxotetrahydropyrimidin-1(2H)-yl)-1,3- dioxoisoindolin-5-yl)methyl)piperazin-1- yl)piperidin-1-yl)pyridin-2-yl)amino)-N,N- dimethyl-7H-pyrrolo[2,3-d]pyrimidine-6- carboxamide |
| GT- 0519 7 | 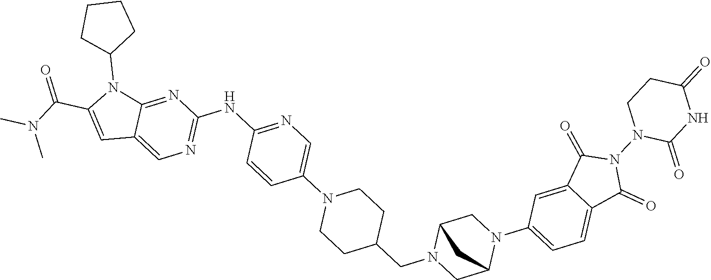 [SMILES: CN(C)C(=O)c1cc2cnc(Nc3ccc(N4CCC(CN5C[C@@H]6C[C@H]5CN6c5ccc6c(c5)C(=O)N(N5CCC(=O)NC5=O)C6=O)CC4)cn3)nc2n1C1CCCC1] | 7-cyclopentyl-2-((5-(4-(((1S,4S)-5-(2-(2,4- dioxotetrahydropyrimidin-1(2H)-yl)-1,3- dioxoisoindolin-5-yl)-2,5- diazabicyclo[2.2.1]heptan-2- yl)methyl)piperidin-1-yl)pyridin-2-yl)amino)- N,N-dimethyl-7H-pyrrolo[2,3-d]pyrimidine- 6-carboxamide |
| GT- 0519 8 | 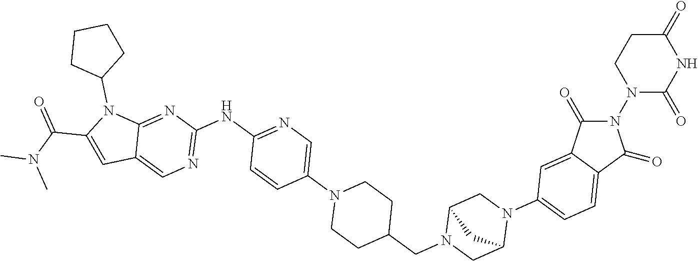 [SMILES: CN(C)C(=O)c1cc2cnc(Nc3ccc(N4CCC(CN5C[C@H]6C[C@@H]5CN6c5ccc6c(c5)C(=O)N(N5CCC(=O)NC5=O)C6=O)CC4)cn3)nc2n1C1CCCC1] | 7-cyclopentyl-2-((5-(4-(((1R,4R)-5-(2-(2,4- dioxotetrahydropyrimidin-1(2H)-yl)-1,3- dioxoisoindolin-5-yl)-2,5- diazabicyclo[2.2.1]heptan-2- yl)methyl)piperidin-1-yl)pyridin-2-yl)amino)- N,N-dimethyl-7H-pyrrolo[2,3-d]pyrimidine- 6-carboxamide |
| GT- 0519 9 | 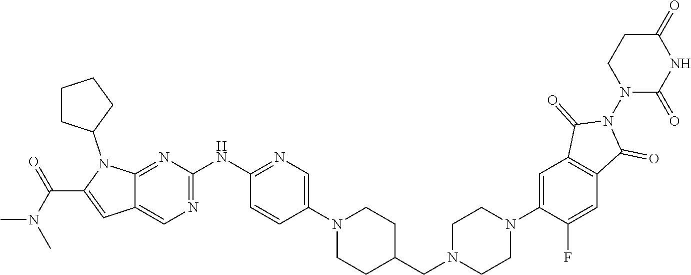 [SMILES: CN(C)C(=O)c1cc2cnc(Nc3ccc(N4CCC(CN5CCN(c6cc7c(cc6F)C(=O)N(N6CCC(=O)NC6=O)C7=O)CC5)CC4)cn3)nc2n1C1CCCC1] | 7-cyclopentyl-2-((5-(4-((4-(2-(2,4- dioxotetrahydropyrimidin-1(2H)-yl)-6-fluoro- 1,3-dioxoisoindolin-5-yl)piperazin-1- yl)methyl)piperidin-1-yl)pyridin-2-yl)amino)- N,N-dimethyl-7H-pyrrolo[2,3-d]pyrimidine- 6-carboxamide |
| GT- 0520 0 | 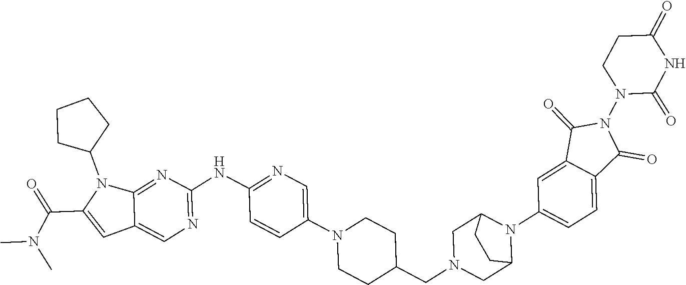 [SMILES: CN(C)C(=O)c1cc2cnc(Nc3ccc(N4CCC(CN5CC6CCC(C5)N6c5ccc6c(c5)C(=O)N(N5CCC(=O)NC5=O)C6=O)CC4)cn3)nc2n1C1CCCC1] | 7-cyclopentyl-2-((5-(4-((8-(2-(2,4- dioxotetrahydropyrimidin-1(2H)-yl)-1,3- dioxoisoindolin-5-yl)-3,8- diazabicyclo[3.2.1]octan-3- yl)methyl)piperidin-1-yl)pyridin-2-yl)amino)- N,N-dimethyl-7H-pyrrolo[2,3-d]pyrimidine- 6-carboxamide |
| GT- 0520 1 | 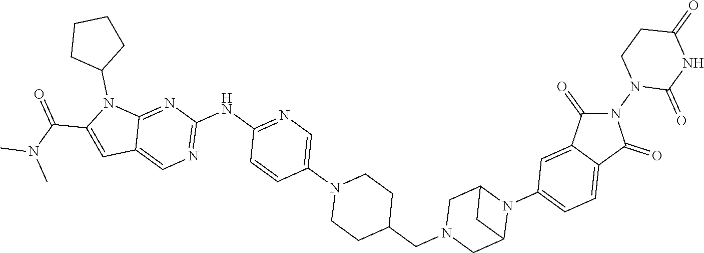 [SMILES: CN(C)C(=O)c1cc2cnc(Nc3ccc(N4CCC(CN5CC6CC(C5)N6c5ccc6c(c5)C(=O)N(N5CCC(=O)NC5=O)C6=O)CC4)cn3)nc2n1C1CCCC1] | 7-cyclopentyl-2-((5-(4-((6-(2-(2,4- dioxotetrahydropyrimidin-1(2H)-yl)-1,3- dioxoisoindolin-5-yl)-3,6- diazabicyclo[3.1.1]heptan-3- yl)methyl)piperidin-1-yl)pyridin-2-yl)amino)- N,N-dimethyl-7H-pyrrolo[2,3-d]pyrimidine- 6-carboxamide |
| GT- 0520 2 | 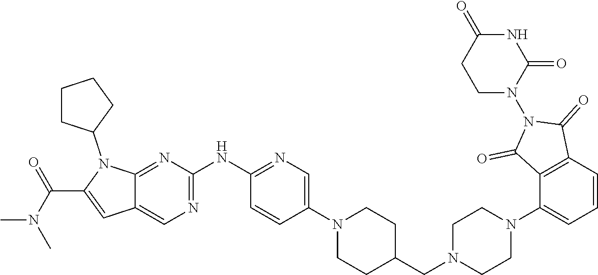 [SMILES: CN(C)C(=O)c1cc2cnc(Nc3ccc(N4CCC(CN5CCN(c6cccc7c6C(=O)N(N6CCC(=O)NC6=O)C7=O)CC5)CC4)cn3)nc2n1C1CCCC1] | 7-cyclopentyl-2-((5-(4-((4-(2-(2,4- dioxotetrahydropyrimidin-1(2H)-yl)-1,3- dioxoisoindolin-4-yl)piperazin-1- yl)methyl)piperidin-1-yl)pyridin-2-yl)amino)- N,N-dimethyl-7H-pyrrolo[2,3-d]pyrimidine- 6-carboxamide |
| GT- 0520 3 | 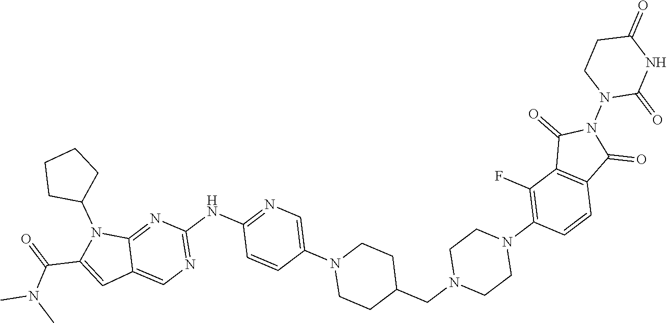 [SMILES: CN(C)C(=O)c1cc2cnc(Nc3ccc(N4CCC(CN5CCN(c6ccc7c(c6F)C(=O)N(N6CCC(=O)NC6=O)C7=O)CC5)CC4)cn3)nc2n1C1CCCC1] | 7-cyclopentyl-2-((5-(4-((4-(2-(2,4- dioxotetrahydropyrimidin-1(2H)-yl)-4-fluoro- 1,3-dioxoisoindolin-5-yl)piperazin-1- yl)methyl)piperidin-1-yl)pyridin-2-yl)amino)- N,N-dimethyl-7H-pyrrolo[2,3-d]pyrimidine- 6-carboxamide |
| GT- 0520 4 | 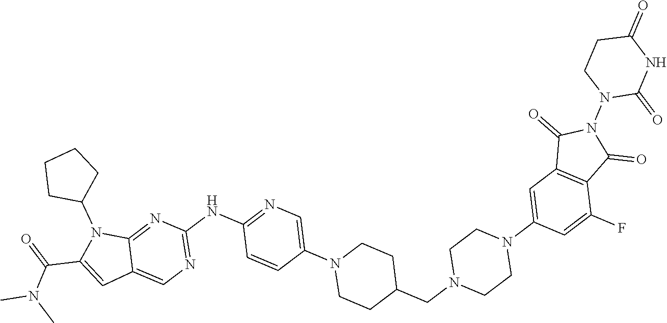 [SMILES: CN(C)C(=O)c1cc2cnc(Nc3ccc(N4CCC(CN5CCN(c6cc(F)c7c(c6)C(=O)N(N6CCC(=O)NC6=O)C7=O)CC5)CC4)cn3)nc2n1C1CCCC1] | 7-cyclopentyl-2-((5-(4-((4-(2-(2,4- dioxotetrahydropyrimidin-1(2H)-yl)-7-fluoro- 1,3-dioxoisoindolin-5-yl)piperazin-1- yl)methyl)piperidin-1-yl)pyridin-2-yl)amino)- N,N-dimethyl-7H-pyrrolo[2,3-d]pyrimidine- 6-carboxamide |
| GT- 0520 5 | 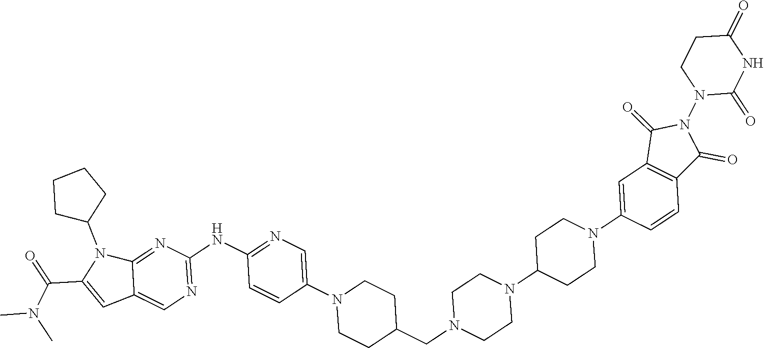 [SMILES: CN(C)C(=O)c1cc2cnc(Nc3ccc(N4CCC(CN5CCN(C6CCN(c7ccc8c(c7)C(=O)N(N7CCC(=O)NC7=O)C8=O)CC6)CC5)CC4)cn3)nc2n1C1CCCC1] | 7-cyclopentyl-2-((5-(4-((4-(1-(2-(2,4- dioxotetrahydropyrimidin-1(2H)-yl)-1,3- dioxoisoindolin-5-yl)piperidin-4-yl)piperazin- 1-yl)methyl)piperidin-1-yl)pyridin-2- yl)amino)-N,N-dimethyl-7H-pyrrolo[2,3- d]pyrimidine-6-carboxamide |
| GT- 0520 6 | 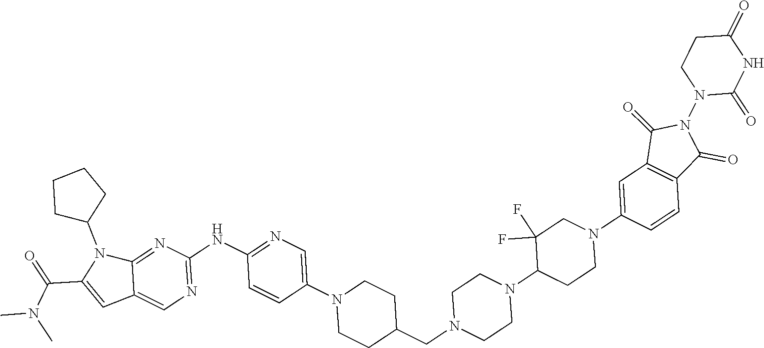 [SMILES: CN(C)C(=O)c1cc2cnc(Nc3ccc(N4CCC(CN5CCN(C6CCN(c7ccc8c(c7)C(=O)N(N7CCC(=O)NC7=O)C8=O)CC6(F)F)CC5)CC4)cn3)nc2n1C1CCCC1] | 7-cyclopentyl-2-((5-(4-((4-(1-(2-(2,4- dioxotetrahydropyrimidin-1(2H)-yl)-1,3- dioxoisoindolin-5-yl)-3,3-difluoropiperidin-4- yl)piperazin-1-yl)methyl)piperidin-1- yl)pyridin-2-yl)amino)-N,N-dimethyl-7H- pyrrolo[2,3-d]pyrimidine-6-carboxamide |
| GT- 0666 1 | 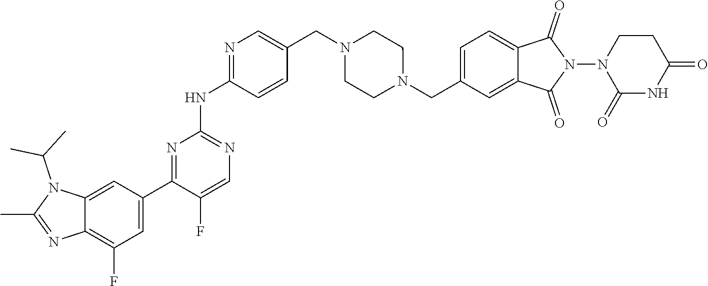 [SMILES: Cc1nc2c(F)cc(-c3nc(Nc4ccc(CN5CCN(Cc6ccc7c(c6)C(=O)N(N6CCC(=O)NC6=O)C7=O)CC5)cn4)ncc3F)cc2n1C(C)C] | 2-(2,4-dioxotetrahydropyrimidin-1(2H)-yl)-5- ((4-((6-((5-fluoro-4-(4-fluoro-1-isopropyl-2- methyl-1H-benzo[d]imidazol-6-yl)pyrimidin- 2-yl)amino)pyridin-3-yl)methyl)piperazin-1- yl)methyl)isoindoline-1,3-dione |
| GT- 0667 3 | 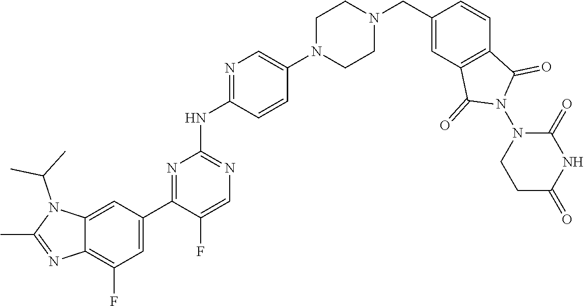 [SMILES: Cc1nc2c(F)cc(-c3nc(Nc4ccc(N5CCN(Cc6ccc7c(c6)C(=O)N(N6CCC(=O)NC6=O)C7=O)CC5)cn4)ncc3F)cc2n1C(C)C] | 2-(2,4-dioxotetrahydropyrimidin-1(2H)-yl)-5- ((4-(6-((5-fluoro-4-(4-fluoro-1-isopropyl-2- methyl-1H-benzo[d]imidazol-6-yl)pyrimidin- 2-yl)amino)pyridin-3-yl)piperazin-1- yl)methyl)isoindoline-1,3-dione |
| GT- 0673 2 | 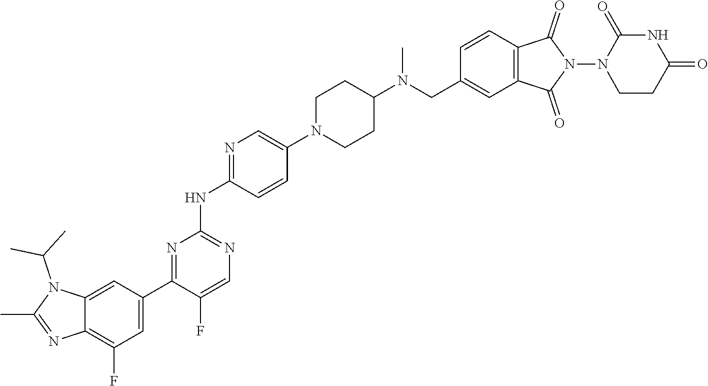 [SMILES: Cc1nc2c(F)cc(-c3nc(Nc4ccc(N5CCC(N(C)Cc6ccc7c(c6)C(=O)N(N6CCC(=O)NC6=O)C7=O)CC5)cn4)ncc3F)cc2n1C(C)C] | 2-(2,4-dioxotetrahydropyrimidin-1(2H)-yl)-5- (((1-(6-((5-fluoro-4-(4-fluoro-1-isopropyl-2- methyl-1H-benzo[d]imidazol-6-yl)pyrimidin- 2-yl)amino)pyridin-3-yl)piperidin-4- yl)(methyl)amino)methyl)isoindoline-1,3- dione |
| GT- 0673 7 | 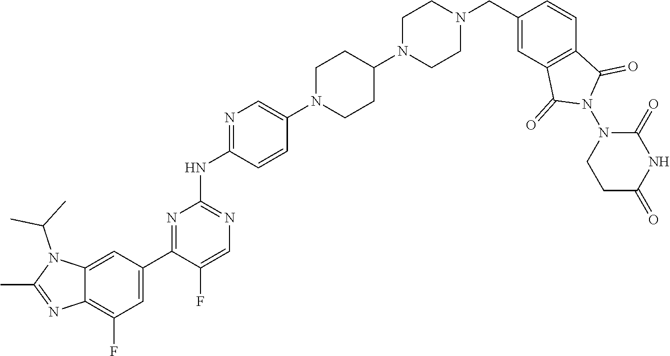 [SMILES: Cc1nc2c(F)cc(-c3nc(Nc4ccc(N5CCC(N6CCN(Cc7ccc8c(c7)C(=O)N(N7CCC(=O)NC7=O)C8=O)CC6)CC5)cn4)ncc3F)cc2n1C(C)C] | 2-(2,4-dioxotetrahydropyrimidin-1(2H)-yl)-5- ((4-(1-(6-((5-fluoro-4-(4-fluoro-1-isopropyl- 2-methyl-1H-benzo[d]imidazol-6- yl)pyrimidin-2-yl)amino)pyridin-3- yl)piperidin-4-yl)piperazin-1- yl)methyl)isoindoline-1,3-dione |
| GT- 0657 4 | 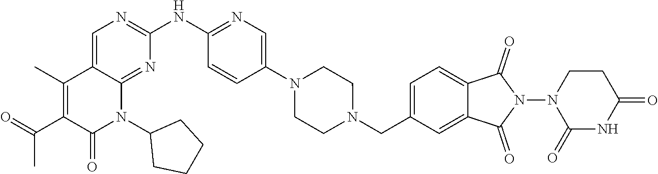 [SMILES: CC(=O)c1c(C)c2cnc(Nc3ccc(N4CCN(Cc5ccc6c(c5)C(=O)N(N5CCC(=O)NC5=O)C6=O)CC4)cn3)nc2n(C2CCCC2)c1=O] | 5-((4-(6-((6-acetyl-8-cyclopentyl-5-methyl-7- oxo-7,8-dihydropyrido[2,3-d]pyrimidin-2- yl)amino)pyridin-3-yl)piperazin-1- yl)methyl)-2-(2,4-dioxotetrahydropyrimidin- 1(2H)-yl)isoindoline-1,3-dione |
| GT- 0658 7 | 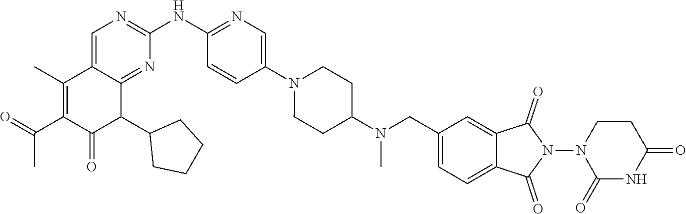 [SMILES: CC(=O)C1=C(C)c2cnc(Nc3ccc(N4CCC(N(C)Cc5ccc6c(c5)C(=O)N(N5CCC(=O)NC5=O)C6=O)CC4)cn3)nc2C(C2CCCC2)C1=O] | 5-(((1-(6-((6-acetyl-8-cyclopentyl-5-methyl- 7-oxo-7,8-dihydropyrido[2,3-d]pyrimidin-2- yl)amino)pyridin-3-yl)piperidin-4- yl)(methyl)amino)methyl)-2-(2,4- dioxotetrahydropyrimidin-1(2H)- yl)isoindoline-1,3-dione |
| GT- 0658 3 | 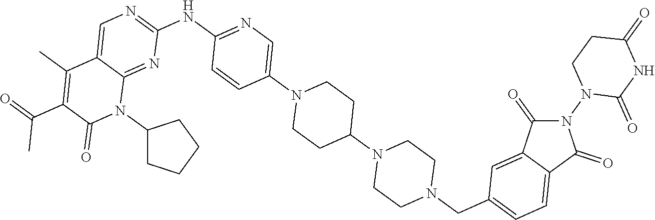 [SMILES: CC(=O)c1c(C)c2cnc(Nc3ccc(N4CCC(N5CCN(Cc6ccc7c(c6)C(=O)N(N6CCC(=O)NC6=O)C7=O)CC5)CC4)cn3)nc2n(C2CCCC2)c1=O] | 5-((4-(1-(6-((6-acetyl-8-cyclopentyl-5- methyl-7-oxo-7,8-dihydropyrido[2,3- d]pyrimidin-2-yl)amino)pyridin-3- yl)piperidin-4-yl)piperazin-1-yl)methyl)-2- (2,4-dioxotetrahydropyrimidin-1(2H)- yl)isoindoline-1,3-dione |
| | 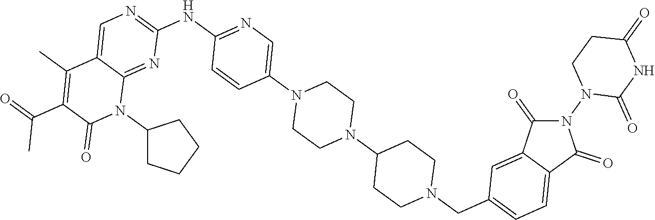 [SMILES: CC(=O)c1c(C)c2cnc(Nc3ccc(N4CCN(C5CCN(Cc6ccc7c(c6)C(=O)N(N6CCC(=O)NC6=O)C7=O)CC5)CC4)cn3)nc2n(C2CCCC2)c1=O] | 5-((4-(4-(6-((6-acetyl-8-cyclopentyl-5- methyl-7-oxo-7,8-dihydropyrido[2,3- d]pyrimidin-2-yl)amino)pyridin-3- yl)piperazin-1-yl)piperidin-1-yl)methyl)-2- (2,4-dioxotetrahydropyrimidin-1(2H)- yl)isoindoline-1,3-dione |
| GT- 0466 0 | 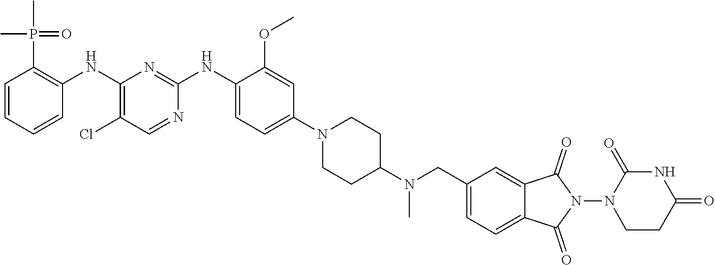 [SMILES: COc1cc(N2CCC(N(C)Cc3ccc4c(c3)C(=O)N(N3CCC(=O)NC3=O)C4=O)CC2)ccc1Nc1ncc(Cl)c(Nc2ccccc2P(C)(C)=O)n1] | 5-(((1-(4-((5-chloro-4-((2- (dimethylphosphoryl)phenyl)amino)pyrimidin- 2-yl)amino)-3-methoxyphenyl)piperidin-4- yl)(methyl)amino)methyl)-2-(2,4- dioxotetrahydropyrimidin-1(2H)- yl)isoindoline-1,3-dione |
| | 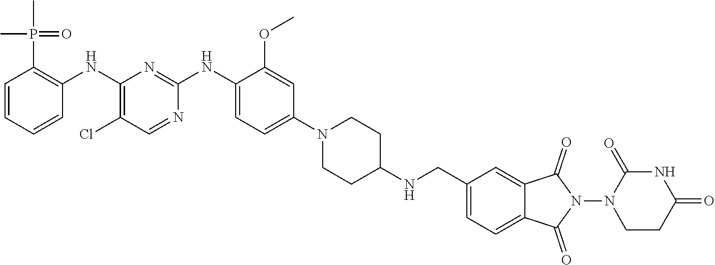 [SMILES: COc1cc(N2CCC(NCc3ccc4c(c3)C(=O)N(N3CCC(=O)NC3=O)C4=O)CC2)ccc1Nc1ncc(Cl)c(Nc2ccccc2P(C)(C)=O)n1] | 5-(((1-(4-((5-chloro-4-((2- (dimethylphosphoryl)phenyl)amino)pyrimidin- 2-yl)amino)-3-methoxyphenyl)piperidin-4- yl)amino)methyl)-2-(2,4- dioxotetrahydropyrimidin-1(2H)- yl)isoindoline-1,3-dione |
| GT- 0734 2 | 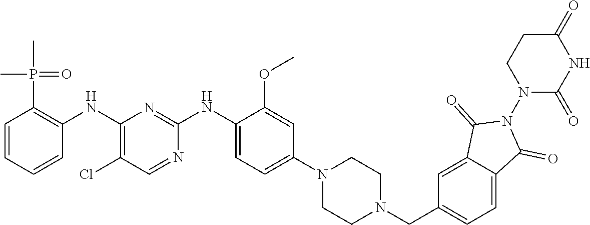 [SMILES: COc1cc(N2CCN(Cc3ccc4c(c3)C(=O)N(N3CCC(=O)NC3=O)C4=O)CC2)ccc1Nc1ncc(Cl)c(Nc2ccccc2P(C)(C)=O)n1] | 5-((4-(4-((5-chloro-4-((2- (dimethylphosphoryl)phenyl)amino)pyrimidin- 2-yl)amino)-3-methoxyphenyl)piperazin-1- yl)methyl)-2-(2,4-dioxotetrahydropyrimidin- 1(2H)-yl)isoindoline-1,3-dione |
| GT- 0814 7 | 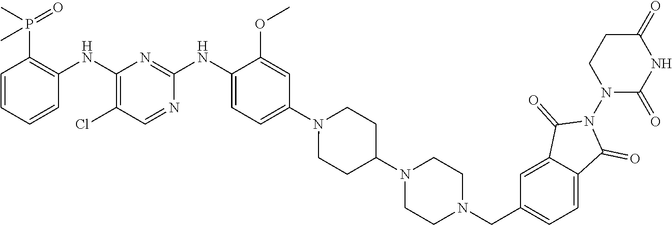 [SMILES: COc1cc(N2CCC(N3CCN(Cc4ccc5c(c4)C(=O)N(N4CCC(=O)NC4=O)C5=O)CC3)CC2)ccc1Nc1ncc(Cl)c(Nc2ccccc2P(C)(C)=O)n1] | 5-((4-(1-(4-((5-chloro-4-((2- (dimethylphosphoryl)phenyl)amino)pyrimidin- 2-yl)amino)-3-methoxyphenyl)piperidin-4- yl)piperazin-1-yl)methyl)-2-(2,4- dioxotetrahydropyrimidin-1(2H)- yl)isoindoline-1,3-dione |
| GT- 0466 1 | 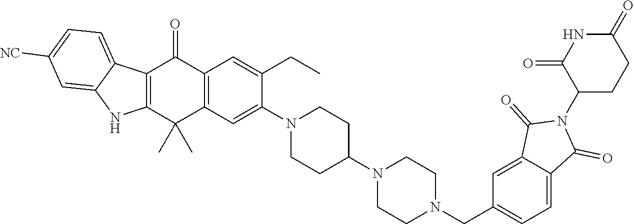 [SMILES: CCc1cc2c(cc1N1CCC(N3CCN(Cc4ccc5c(c4)C(=O)N(C4CCC(=O)NC4=O)C5=O)CC3)CC1)C(C)(C)c1[nH]c3cc(C#N)ccc3c1C2=O] | 8-(4-(4-((2-(2,4-dioxotetrahydropyrimidin- 1(2H)-yl)-1,3-dioxoisoindolin-5- yl)methyl)piperazin-1-yl)piperidin-1-yl)-9- ethyl-6,6-dimethyl-11-oxo-6,11-dihydro-5H- benzo[b]carbazole-3-carbonitrile |
| GT- 0732 0 | 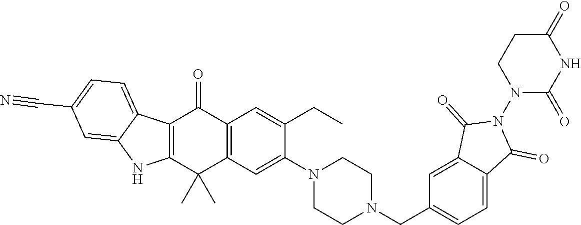 [SMILES: CCc1cc2c(cc1N1CCN(Cc3ccc4c(c3)C(=O)N(N3CCC(=O)NC3=O)C4=O)CC1)C(C)(C)c1[nH]c3cc(C#N)ccc3c1C2=O] | 8-(4-((2-(2,4-dioxotetrahydropyrimidin- 1(2H)-yl)-1,3-dioxoisoindolin-5- yl)methyl)piperazin-1-yl)-9-ethyl-6,6- dimethyl-11-oxo-6,11-dihydro-5H- benzo[b]carbazole-3-carbonitrile |
| | 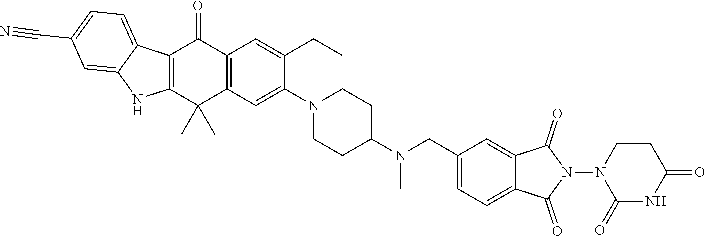 [SMILES: CCc1cc2c(cc1N1CCC(N(C)Cc3ccc4c(c3)C(=O)N(N3CCC(=O)NC3=O)C4=O)CC1)C(C)(C)c1[nH]c3cc(C#N)ccc3c1C2=O] | 8-(4-(((2-(2,4-dioxotetrahydropyrimidin- 1(2H)-yl)-1,3-dioxoisoindolin-5- yl)methyl)(methyl)amino)piperidin-1-yl)-9- ethyl-6,6-dimethyl-11-oxo-6,11-dihydro-5H- benzo[b]carbazole-3-carbonitrile |
| GT- 0465 9 | 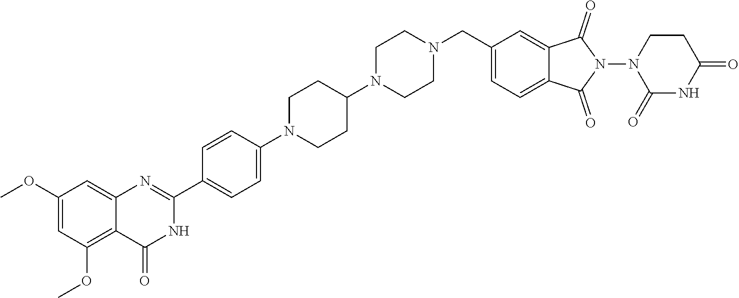 [SMILES: COc1cc(OC)c2c(=O)[nH]c(-c3ccc(N4CCC(N5CCN(Cc6ccc7c(c6)C(=O)N(N6CCC(=O)NC6=O)C7=O)CC5)CC4)cc3)nc2c1] | 5-((4-(1-(4-(5,7-dimethoxy-4-oxo-3,4- dihydroquinazolin-2-yl)phenyl)piperidin-4- yl)piperazin-1-yl)methyl)-2-(2,4- dioxotetrahydropyrimidin-1(2H)- yl)isoindoline-1,3-dione |
| | 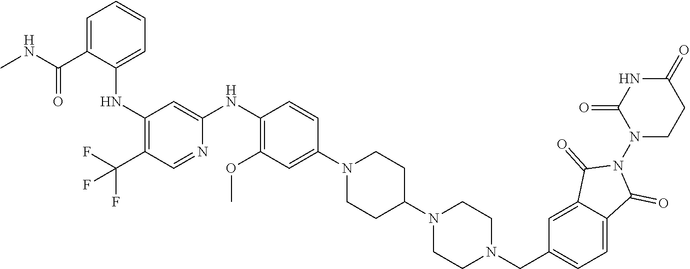 [SMILES: CNC(=O)c1ccccc1Nc1cc(Nc2ccc(N3CCC(N4CCN(Cc5ccc6c(c5)C(=O)N(N5CCC(=O)NC5=O)C6=O)CC4)CC3)cc2OC)ncc1C(F)(F)F] | 2-((2-((4-(4-(4-((2-(2,4- dioxotetrahydropyrimidin-1(2H)-yl)-1,3- dioxoisoindolin-5-yl)methyl)piperazin-1- yl)piperidin-1-yl)-2-methoxyphenyl)amino)- 5-(trifluoromethyl)pyridin-4-yl)amino)-N- methylbenzamide |
| | 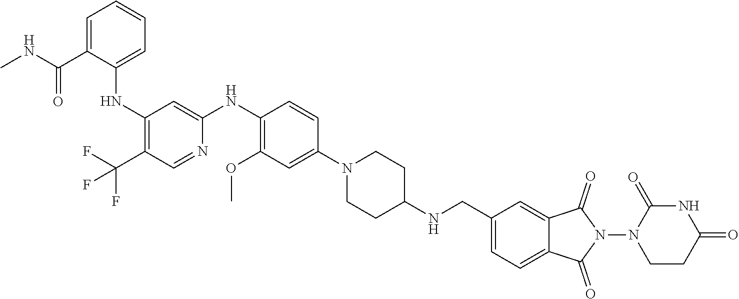 [SMILES: CNC(=O)c1ccccc1Nc1cc(Nc2ccc(N3CCC(NCc4ccc5c(c4)C(=O)N(N4CCC(=O)NC4=O)C5=O)CC3)cc2OC)ncc1C(F)(F)F] | 2-((2-((4-(4-(((2-(2,4- dioxotetrahydropyrimidin-1(2H)-yl)-1,3- dioxoisoindolin-5-yl)methyl)amino)piperidin- 1-yl)-2-methoxyphenyl)amino)-5- (trifluoromethyl)pyridin-4-yl)amino)-N- methylbenzamide |
| GT- 0764 8 | 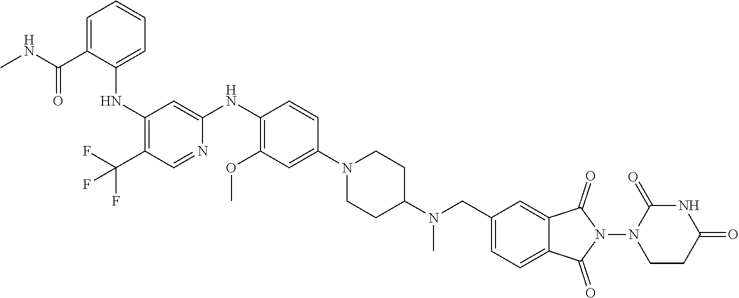 [SMILES: CNC(=O)c1ccccc1Nc1cc(Nc2ccc(N3CCC(N(C)Cc4ccc5c(c4)C(=O)N(N4CCC(=O)NC4=O)C5=O)CC3)cc2OC)ncc1C(F)(F)F] | 2-((2-((4-(4-(((2-(2,4- dioxotetrahydropyrimidin-1(2H)-yl)-1,3- dioxoisoindolin-5- yl)methyl)(methyl)amino)piperidin-1-yl)-2- methoxyphenyl)amino)-5- (trifluoromethyl)pyridin-4-yl)amino)-N- methylbenzamide |
| GT- 0766 1 | 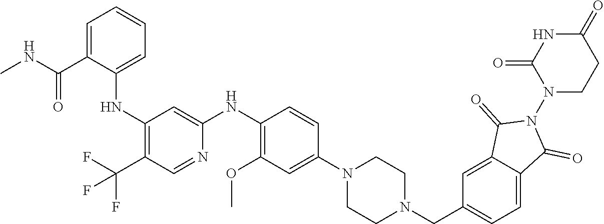 [SMILES: CNC(=O)c1ccccc1Nc1cc(Nc2ccc(N3CCN(Cc4ccc5c(c4)C(=O)N(N4CCC(=O)NC4=O)C5=O)CC3)cc2OC)ncc1C(F)(F)F] | 2-((2-((4-(4-((2-(2,4- dioxotetrahydropyrimidin-1(2H)-yl)-1,3- dioxoisoindolin-5-yl)methyl)piperazin-1-yl)- 2-methoxyphenyl)amino)-5- (trifluoromethyl)pyridin-4-yl)amino)-N- methylbenzamide |
| GT- 0766 4 | 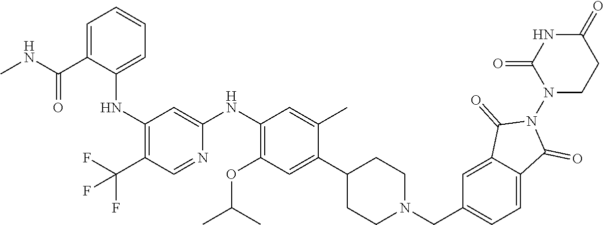 [SMILES: CNC(=O)c1ccccc1Nc1cc(Nc2cc(C)c(C3CCN(Cc4ccc5c(c4)C(=O)N(N4CCC(=O)NC4=O)C5=O)CC3)cc2OC(C)C)ncc1C(F)(F)F] | 2-((2-((4-(1-((2-(2,4- dioxotetrahydropyrimidin-1(2H)-yl)-1,3- dioxoisoindolin-5-yl)methyl)piperidin-4-yl)- 2-isopropoxy-5-methylphenyl)amino)-5- (trifluoromethyl)pyridin-4-yl)amino)-N- methylbenzamide |
| | 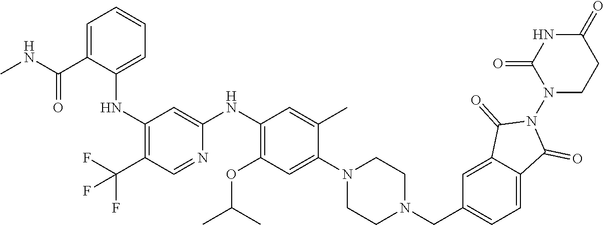 [SMILES: CNC(=O)c1ccccc1Nc1cc(Nc2cc(C)c(N3CCN(Cc4ccc5c(c4)C(=O)N(N4CCC(=O)NC4=O)C5=O)CC3)cc2OC(C)C)ncc1C(F)(F)F] | 2-((2-((4-(4-((2-(2,4- dioxotetrahydropyrimidin-1(2H)-yl)-1,3- dioxoisoindolin-5-yl)methyl)piperazin-1-yl)- 2-isopropoxy-5-methylphenyl)amino)-5- (trifluoromethyl)pyridin-4-yl)amino)-N- methylbenzamide |
| | 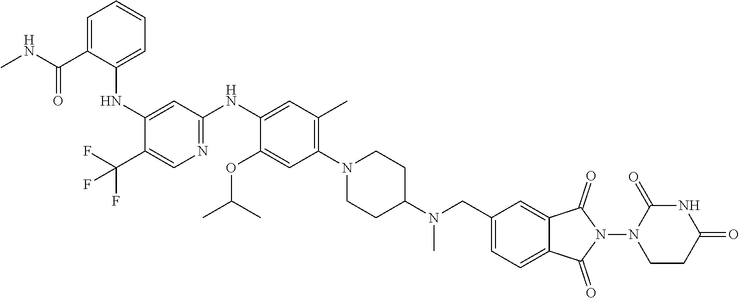 [SMILES: CNC(=O)c1ccccc1Nc1cc(Nc2cc(C)c(N3CCC(N(C)Cc4ccc5c(c4)C(=O)N(N4CCC(=O)NC4=O)C5=O)CC3)cc2OC(C)C)ncc1C(F)(F)F] | 2-((2-((4-(4-(((2-(2,4- dioxotetrahydropyrimidin-1(2H)-yl)-1,3- dioxoisoindolin-5- yl)methyl)(methyl)amino)piperidin-1-yl)-2- isopropoxy-5-methylphenyl)amino)-5- (trifluoromethyl)pyridin-4-yl)amino)-N- methylbenzamide |
| | 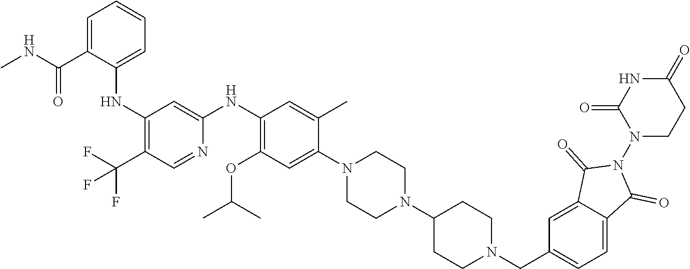 [SMILES: CNC(=O)c1ccccc1Nc1cc(Nc2cc(C)c(N3CCN(C4CCN(Cc5ccc6c(c5)C(=O)N(N5CCC(=O)NC5=O)C6=O)CC4)CC3)cc2OC(C)C)ncc1C(F)(F)F] | 2-((2-((4-(4-(1-((2-(2,4- dioxotetrahydropyrimidin-1(2H)-yl)-1,3- dioxoisoindolin-5-yl)methyl)piperidin-4- yl)piperazin-1-yl)-2-isopropoxy-5- methylphenyl)amino)-5- (trifluoromethyl)pyridin-4-yl)amino)-N- methylbenzamide |
| | 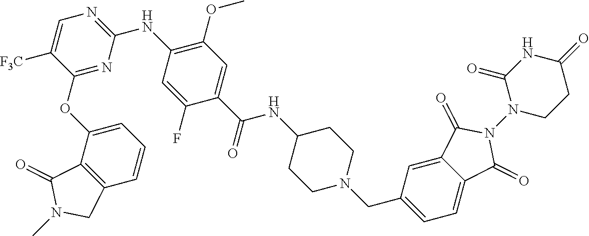 [SMILES: COc1cc(C(=O)NC2CCN(Cc3ccc4c(c3)C(=O)N(N3CCC(=O)NC3=O)C4=O)CC2)c(F)cc1Nc1ncc(C(F)(F)F)c(Oc2cccc3c2C(=O)N(C)C3)n1] | N-(1-((2-(2,4-dioxotetrahydropyrimidin- 1(2H)-yl)-1,3-dioxoisoindolin-5- yl)methyl)piperidin-4-yl)-2-fluoro-5- methoxy-4-((4-((2-methyl-3-oxoisoindolin-4- yl)oxy)-5-(trifluoromethyl)pyrimidin-2- yl)amino)benzamide |
| GT- 0750 5 | 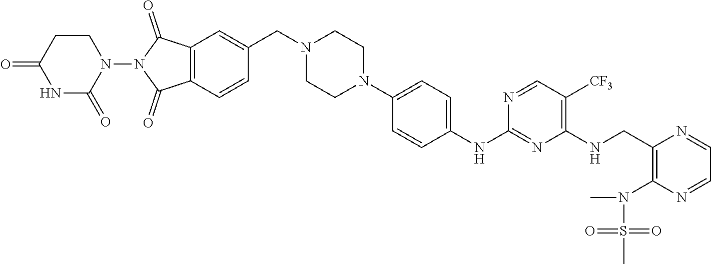 [SMILES: CN(c1nccnc1CNc1nc(Nc2ccc(N3CCN(Cc4ccc5c(c4)C(=O)N(N4CCC(=O)NC4=O)C5=O)CC3)cc2)ncc1C(F)(F)F)S(C)(=O)=O] | N-(3-(((2-((4-(4-((2-(2,4- dioxotetrahydropyrimidin-1(2H)-yl)-1,3- dioxoisoindolin-5-yl)methyl)piperazin-1- yl)phenyl)amino)-5- (trifluoromethyl)pyrimidin-4- yl)amino)methyl)pyrazin-2-yl)-N- methylmethanesulfonamide |
| GT- 0813 7 | 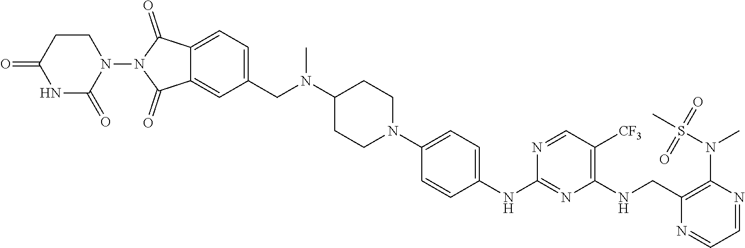 [SMILES: CN(Cc1ccc2c(c1)C(=O)N(N1CCC(=O)NC1=O)C2=O)C1CCN(c2ccc(Nc3ncc(C(F)(F)F)c(NCc4nccnc4N(C)S(C)(=O)=O)n3)cc2)CC1] | N-(3-(((2-((4-(4-(((2-(2,4- dioxotetrahydropyrimidin-1(2H)-yl)-1,3- dioxoisoindolin-5- yl)methyl)(methyl)amino)piperidin-1- yl)phenyl)amino)-5- (trifluoromethyl)pyrimidin-4- yl)amino)methyl)pyrazin-2-yl)-N- methylmethanesulfonamide |
| GT- 0813 9 | 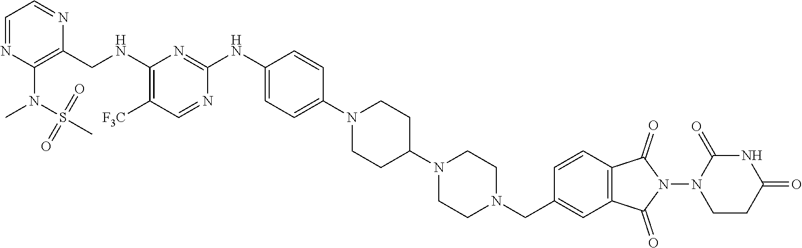 [SMILES: CN(c1nccnc1CNc1nc(Nc2ccc(N3CCC(N4CCN(Cc5ccc6c(c5)C(=O)N(N5CCC(=O)NC5=O)C6=O)CC4)CC3)cc2)ncc1C(F)(F)F)S(C)(=O)=O] | N-(3-(((2-((4-(4-(4-((2-(2,4- dioxotetrahydropyrimidin-1(2H)-yl)-1,3- dioxoisoindolin-5-yl)methyl)piperazin-1- yl)piperidin-1-yl)phenyl)amino)-5- (trifluoromethyl)pyrimidin-4- yl)amino)methyl)pyrazin-2-yl)-N- methylmethanesulfonamide |
| GT- 0814 1 | 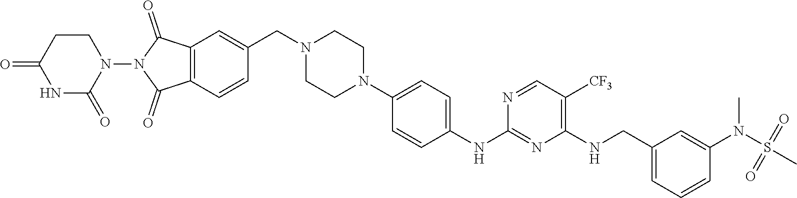 [SMILES: CN(c1cccc(CNc2nc(Nc3ccc(N4CCN(Cc5ccc6c(c5)C(=O)N(N5CCC(=O)NC5=O)C6=O)CC4)cc3)ncc2C(F)(F)F)c1)S(C)(=O)=O] | N-(3-(((2-((4-(4-((2-(2,4- dioxotetrahydropyrimidin-1(2H)-yl)-1,3- dioxoisoindolin-5-yl)methyl)piperazin-1- yl)phenyl)amino)-5- (trifluoromethyl)pyrimidin-4- yl)amino)methyl)phenyl)-N- methylmethanesulfonamide |
| | 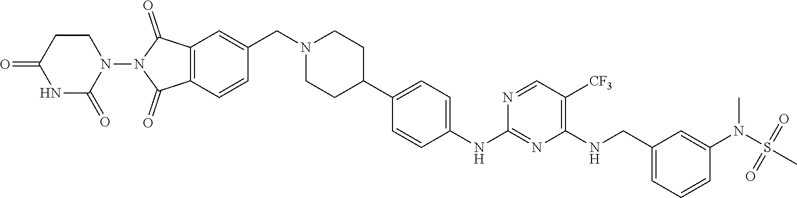 [SMILES: CN(c1cccc(CNc2nc(Nc3ccc(C4CCN(Cc5ccc6c(c5)C(=O)N(N5CCC(=O)NC5=O)C6=O)CC4)cc3)ncc2C(F)(F)F)c1)S(C)(=O)=O] | N-(3-(((2-((4-(1-((2-(2,4- dioxotetrahydropyrimidin-1(2H)-yl)-1,3- dioxoisoindolin-5-yl)methyl)piperidin-4- yl)phenyl)amino)-5- (trifluoromethyl)pyrimidin-4- yl)amino)methyl)phenyl)-N- methylmethanesulfonamide |
| GT- 0814 3 | 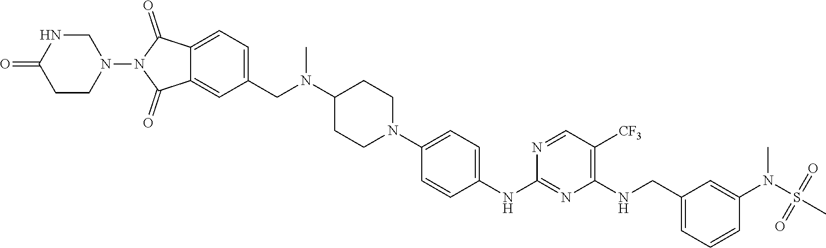 [SMILES: CN(Cc1ccc2c(c1)C(=O)N(N1CCC(=O)NC1)C2=O)C1CCN(c2ccc(Nc3ncc(C(F)(F)F)c(NCc4cccc(N(C)S(C)(=O)=O)c4)n3)cc2)CC1] | N-(3-(((2-((4-(4-(((2-(2,4- dioxotetrahydropyrimidin-1(2H)-yl)-1,3- dioxoisoindolin-5- yl)methyl)(methyl)amino)piperidin-1- yl)phenyl)amino)-5- (trifluoromethyl)pyrimidin-4- yl)amino)methyl)phenyl)-N- methylmethanesulfonamide |
| GT- 0814 5 | 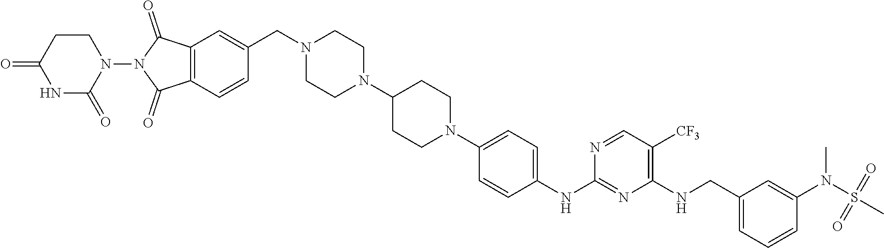 [SMILES: CN(c1cccc(CNc2nc(Nc3ccc(N4CCC(N5CCN(Cc6ccc7c(c6)C(=O)N(N6CCC(=O)NC6=O)C7=O)CC5)CC4)cc3)ncc2C(F)(F)F)c1)S(C)(=O)=O] | N-(3-(((2-((4-(4-(4-((2-(2,4- dioxotetrahydropyrimidin-1(2H)-yl)-1,3- dioxoisoindolin-5-yl)methyl)piperazin-1- yl)piperidin-1-yl)phenyl)amino)-5- (trifluoromethyl)pyrimidin-4- yl)amino)methyl)phenyl)-N- methylmethanesulfonamide |
| GT- 0751 5 | 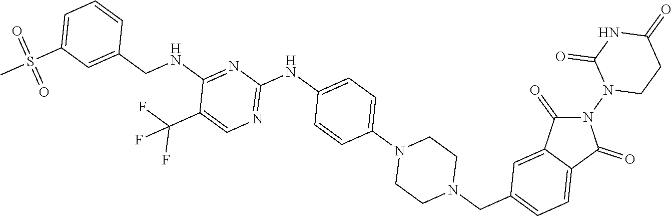 [SMILES: CS(=O)(=O)c1cccc(CNc2nc(Nc3ccc(N4CCN(Cc5ccc6c(c5)C(=O)N(N5CCC(=O)NC5=O)C6=O)CC4)cc3)ncc2C(F)(F)F)c1] | 2-(2,4-dioxotetrahydropyrimidin-1(2H)-yl)-5- ((4-(4-((4-((3- (methylsulfonyl)benzyl)amino)-5- (trifluoromethyl)pyrimidin-2- yl)amino)phenyl)piperazin-1- yl)methyl)isoindoline-1,3-dione |
| GT- 0466 8 | 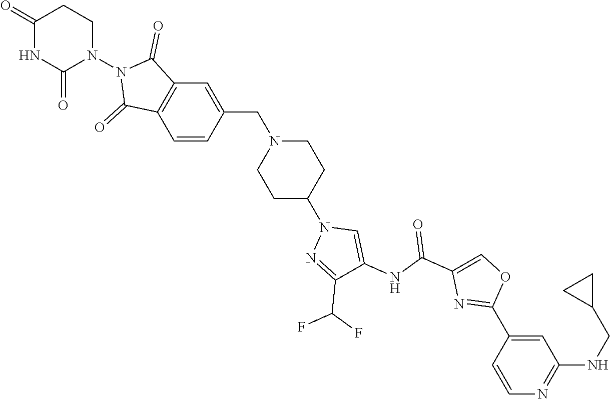 [SMILES: O=C1CCN(N2C(=O)c3ccc(CN4CCC(n5cc(NC(=O)c6coc(-c7ccnc(NCC8CC8)c7)n6)c(C(F)F)n5)CC4)cc3C2=O)C(=O)N1] | 2-(2-((cyclopropylmethyl)amino)pyridin-4- yl)-N-(3-(difluoromethyl)-1-(1-((2-(2,4- dioxotetrahydropyrimidin-1(2H)-yl)-1,3- dioxoisoindolin-5-yl)methyl)piperidin-4-yl)- 1H-pyrazol-4-yl)oxazole-4-carboxamide |
| GT- 0495 3 | 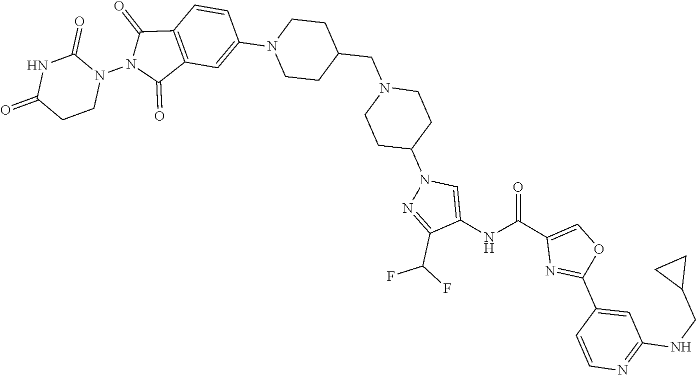 [SMILES: O=C1CCN(N2C(=O)c3ccc(N4CCC(CN5CCC(n6cc(NC(=O)c7coc(-c8ccnc(NCC9CC9)c8)n7)c(C(F)F)n6)CC5)CC4)cc3C2=O)C(=O)N1] | 2-(2-((cyclopropylmethyl)amino)pyridin-4- yl)-N-(3-(difluoromethyl)-1-(1-((1-(2-(2,4- dioxotetrahydropyrimidin-1(2H)-yl)-1,3- dioxoisoindolin-5-yl)piperidin-4- yl)methyl)piperidin-4-yl)-1H-pyrazol-4- yl)oxazole-4-carboxamide |
| GT- 0513 2 | 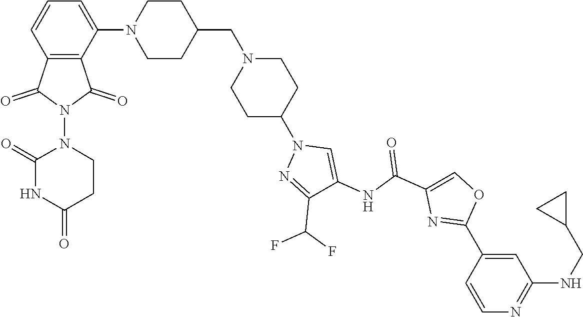 [SMILES: O=C1CCN(N2C(=O)c3cccc(N4CCC(CN5CCC(n6cc(NC(=O)c7coc(-c8ccnc(NCC9CC9)c8)n7)c(C(F)F)n6)CC5)CC4)c3C2=O)C(=O)N1] | 2-(2-((cyclopropylmethyl)amino)pyridin-4- yl)-N-(3-(difluoromethyl)-1-(1-((1-(2-(2,4- dioxotetrahydropyrimidin-1(2H)-yl)-1,3- dioxoisoindolin-4-yl)piperidin-4- yl)methyl)piperidin-4-yl)-1H-pyrazol-4- yl)oxazole-4-carboxamide |
| GT- 0513 3 | 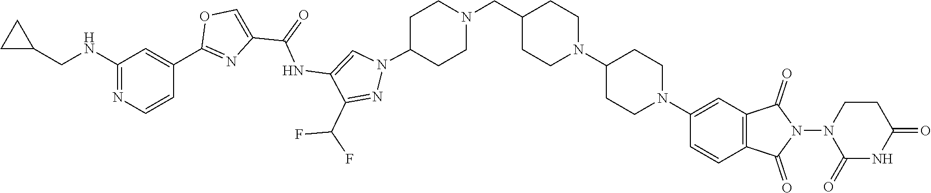 [SMILES: O=C1CCN(N2C(=O)c3ccc(N4CCC(N5CCC(CN6CCC(n7cc(NC(=O)c8coc(-c9ccnc(NCC%10CC%10)c9)n8)c(C(F)F)n7)CC6)CC5)CC4)cc3C2=O)C(=O)N1] | 2-(2-((cyclopropylmethyl)amino)pyridin-4- yl)-N-(3-(difluoromethyl)-1-(1-((1′-(2-(2,4- dioxotetrahydropyrimidin-1(2H)-yl)-1,3- dioxoisoindolin-5-yl)-[1,4′-bipiperidin]-4- yl)methyl)piperidin-4-yl)-1H-pyrazol-4- yl)oxazole-4-carboxamide |
| | 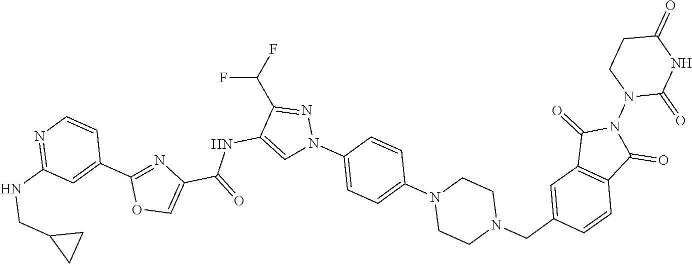 [SMILES: O=C1CCN(N2C(=O)c3ccc(CN4CCN(c5ccc(-n6cc(NC(=O)c7coc(-c8ccnc(NCC9CC9)c8)n7)c(C(F)F)n6)cc5)CC4)cc3C2=O)C(=O)N1] | 2-(2-((cyclopropylmethyl)amino)pyridin-4- yl)-N-(3-(difluoromethyl)-1-(4-(4-((2-(2,4- dioxotetrahydropyrimidin-1(2H)-yl)-1,3- dioxoisoindolin-5-yl)methyl)piperazin-1- yl)phenyl)-1H-pyrazol-4-yl)oxazole-4- carboxamide |
| GT- 0507 8 | 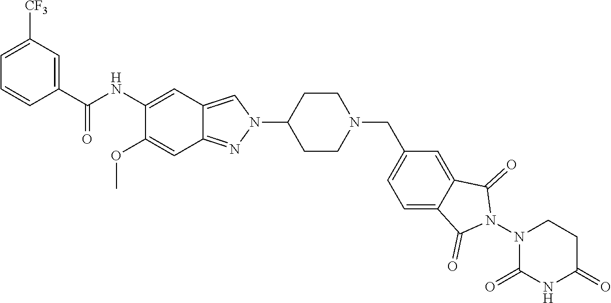 [SMILES: COc1cc2nn(C3CCN(Cc4ccc5c(c4)C(=O)N(N4CCC(=O)NC4=O)C5=O)CC3)cc2cc1NC(=O)c1cccc(C(F)(F)F)c1] | N-(2-(1-((2-(2,4-dioxotetrahydropyrimidin- 1(2H)-yl)-1,3-dioxoisoindolin-5- yl)methyl)piperidin-4-yl)-6-methoxy-2H- indazol-5-yl)-3-(trifluoromethyl)benzamide |
| GT- 0487 7 | 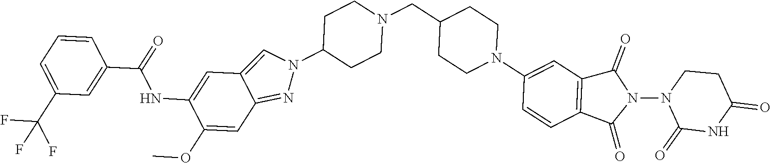 [SMILES: COc1cc2nn(C3CCN(CC4CCN(c5ccc6c(c5)C(=O)N(N5CCC(=O)NC5=O)C6=O)CC4)CC3)cc2cc1NC(=O)c1cccc(C(F)(F)F)c1] | N-(2-(1-((1-(2-(2,4- dioxotetrahydropyrimidin-1(2H)-yl)-1,3- dioxoisoindolin-5-yl)piperidin-4- yl)methyl)piperidin-4-yl)-6-methoxy-2H- indazol-5-yl)-3-(trifluoromethyl)benzamide |
| GT- 0507 9 | 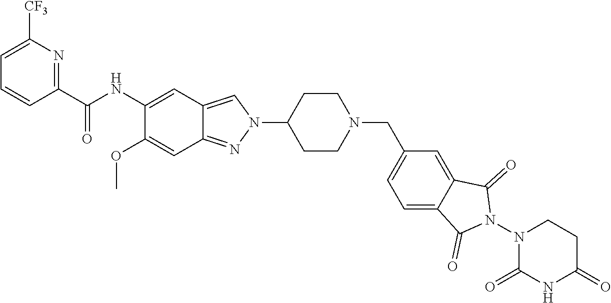 [SMILES: COc1cc2nn(C3CCN(Cc4ccc5c(c4)C(=O)N(N4CCC(=O)NC4=O)C5=O)CC3)cc2cc1NC(=O)c1cccc(C(F)(F)F)n1] | N-(2-(1-((2-(2,4-dioxotetrahydropyrimidin- 1(2H)-yl)-1,3-dioxoisoindolin-5- yl)methyl)piperidin-4-yl)-6-methoxy-2H- indazol-5-yl)-6-(trifluoromethyl)picolinamide |
| GT- 0488 3 | 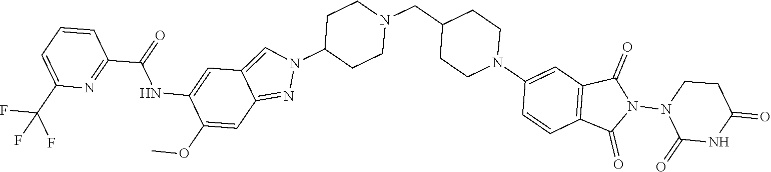 [SMILES: COc1cc2nn(C3CCN(CC4CCN(c5ccc6c(c5)C(=O)N(N5CCC(=O)NC5=O)C6=O)CC4)CC3)cc2cc1NC(=O)c1cccc(C(F)(F)F)n1] | N-(2-(1-((1-(2-(2,4- dioxotetrahydropyrimidin-1(2H)-yl)-1,3- dioxoisoindolin-5-yl)piperidin-4- yl)methyl)piperidin-4-yl)-6-methoxy-2H- indazol-5-yl)-6-(trifluoromethyl)picolinamide |
| GT- 0488 5 | 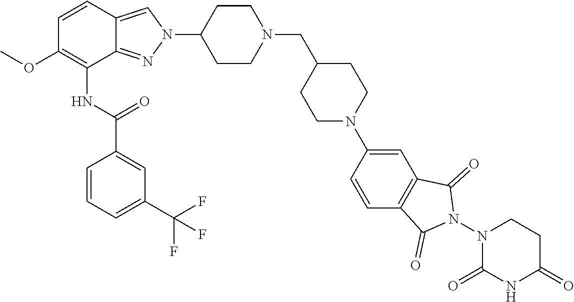 [SMILES: COc1ccc2cn(C3CCN(CC4CCN(c5ccc6c(c5)C(=O)N(N5CCC(=O)NC5=O)C6=O)CC4)CC3)nc2c1NC(=O)c1cccc(C(F)(F)F)c1] | N-(2-(1-((1-(2-(2,4- dioxotetrahydropyrimidin-1(2H)-yl)-1,3- dioxoisoindolin-5-yl)piperidin-4- yl)methyl)piperidin-4-yl)-6-methoxy-2H- indazol-7-yl)-3-(trifluoromethyl)benzamide |
| GT- 0462 1 | 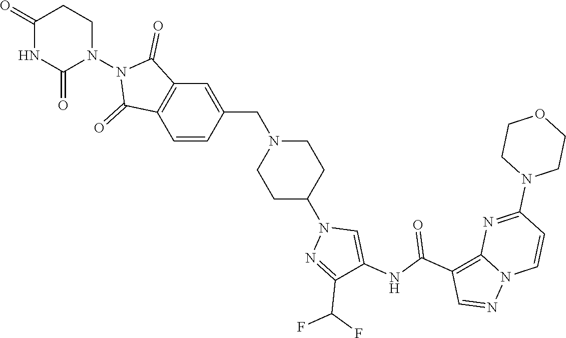 [SMILES: O=C1CCN(N2C(=O)c3ccc(CN4CCC(n5cc(NC(=O)c6cnn7ccc(N8CCOCC8)nc67)c(C(F)F)n5)CC4)cc3C2=O)C(=O)N1] | N-(3-(difluoromethyl)-1-(1-((2-(2,4- dioxotetrahydropyrimidin-1(2H)-yl)-1,3- dioxoisoindolin-5-yl)methyl)piperidin-4-yl)- 1H-pyrazol-4-yl)-5-morpholinopyrazolo[1,5- a]pyrimidine-3-carboxamide |
| GT- 0522 4 | 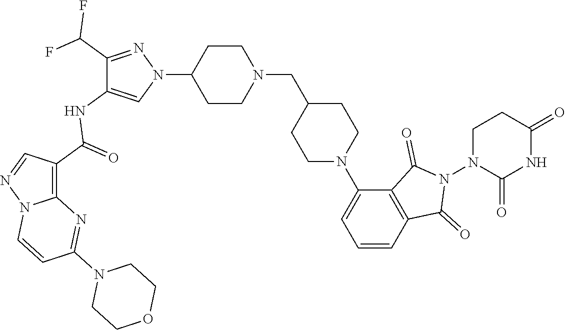 [SMILES: O=C1CCN(N2C(=O)c3cccc(N4CCC(CN5CCC(n6cc(NC(=O)c7cnn8ccc(N9CCOCC9)nc78)c(C(F)F)n6)CC5)CC4)c3C2=O)C(=O)N1] | N-(3-(difluoromethyl)-1-(1-((1-(2-(2,4- dioxotetrahydropyrimidin-1(2H)-yl)-1,3- dioxoisoindolin-4-yl)piperidin-4- yl)methyl)piperidin-4-yl)-1H-pyrazol-4-yl)-5- morpholinopyrazolo[1,5-a]pyrimidine-3- carboxamide |
| GT- 0529 1 | 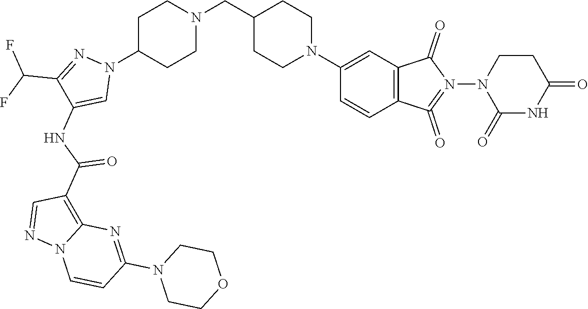 | N-(3-(difluoromethyl)-1-(1-((1-(2-(2,4- dioxotetrahydropyrimidin-1(2H)-yl)-1,3- dioxoisoindolin-5-yl)piperidin-4- yl)methyl)piperidin-4-yl)-1H-pyrazol-4-yl)-5- morpholinopyrazolo[1,5-a]pyrimidine-3- carboxamide |
| GT- 0508 0 | 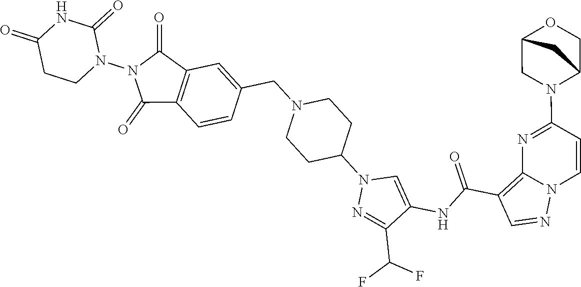 [SMILES: O=C1CCN(N2C(=O)c3ccc(CN4CCC(n5cc(NC(=O)c6cnn7ccc(N8C[C@H]9C[C@@H]8CO9)nc67)c(C(F)F)n5)CC4)cc3C2=O)C(=O)N1] | 5-((1R,4R)-2-oxa-5-azabicyclo[2.2.1]heptan- 5-yl)-N-(3-(difluoromethyl)-1-(1-((2-(2,4- dioxotetrahydropyrimidin-1(2H)-yl)-1,3- dioxoisoindolin-5-yl)methyl)piperidin-4-yl)- 1H-pyrazol-4-yl)pyrazolo[1,5-a]pyrimidine- 3-carboxamide |
| GT- 0505 6 | 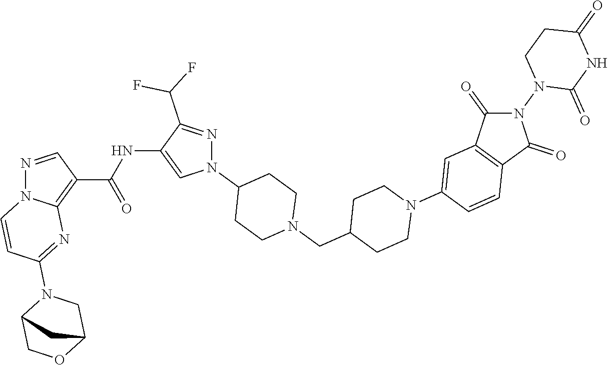 [SMILES: O=C1CCN(N2C(=O)c3ccc(N4CCC(CN5CCC(n6cc(NC(=O)c7cnn8ccc(N9C[C@H]%10C[C@@H]9CO%10)nc78)c(C(F)F)n6)CC5)CC4)cc3C2=O)C(=O)N1] | 5-((1R,4R)-2-oxa-5-azabicyclo[2.2.1]heptan- 5-yl)-N-(3-(difluoromethyl)-1-(1-((1-(2-(2,4- dioxotetrahydropyrimidin-1(2H)-yl)-1,3- dioxoisoindolin-5-yl)piperidin-4- yl)methyl)piperidin-4-yl)-1H-pyrazol-4- yl)pyrazolo[1,5-a]pyrimidine-3-carboxamide |
| GT- 0528 5 | 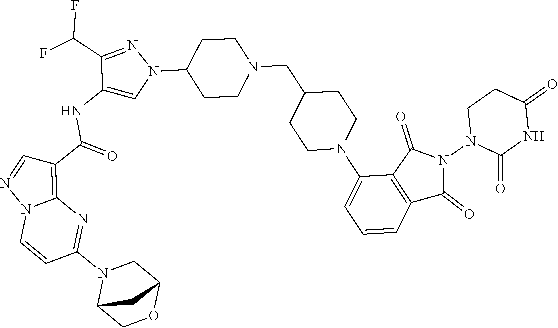 [SMILES: O=C1CCN(N2C(=O)c3cccc(N4CCC(CN5CCC(n6cc(NC(=O)c7cnn8ccc(N9C[C@H]%10C[C@@H]9CO%10)nc78)c(C(F)F)n6)CC5)CC4)c3C2=O)C(=O)N1] | 5-((1R,4R)-2-oxa-5-azabicyclo[2.2.1]heptan- 5-yl)-N-(3-(difluoromethyl)-1-(1-((1-(2-(2,4- dioxotetrahydropyrimidin-1(2H)-yl)-1,3- dioxoisoindolin-4-yl)piperidin-4- yl)methyl)piperidin-4-yl)-1H-pyrazol-4- yl)pyrazolo[1,5-a]pyrimidine-3-carboxamide |
| GT- 0523 6 | 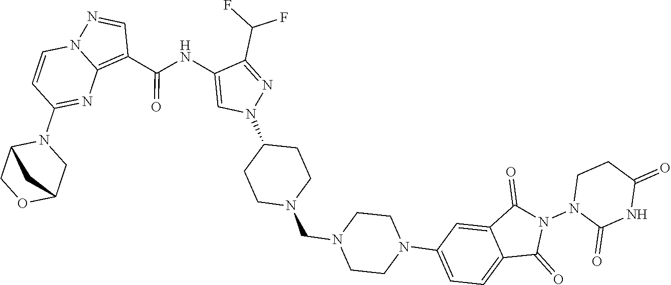 | 5-((1R,4R)-2-oxa-5-azabicyclo[2.2.1]heptan- 5-yl)-N-(3-(difluoromethyl)-1-((1r,4R)-4-((4- (2-(2,4-dioxotetrahydropyrimidin-1(2H)-yl)- 1,3-dioxoisoindolin-5-yl)piperazin-1- yl)methyl)cyclohexyl)-1H-pyrazol-4- yl)pyrazolo[1,5-a]pyrimidine-3-carboxamide |
| GT- 0523 7 | 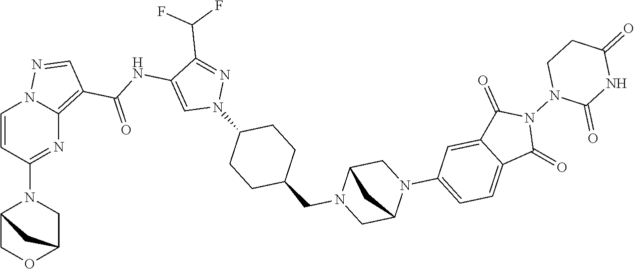 [SMILES: O=C1CCN(N2C(=O)c3ccc(N4C[C@@H]5C[C@H]4CN5C[C@H]4CC[C@H](n5cc(NC(=O)c6cnn7ccc(N8C[C@H]9C[C@@H]8CO9)nc67)c(C(F)F)n5)CC4)cc3C2=O)C(=O)N1] | 5-((1R,4R)-2-oxa-5-azabicyclo[2.2.1]heptan- 5-yl)-N-(3-(difluoromethyl)-1-((1S,4R)-4- (((1S,4S)-5-(2-(2,4- dioxotetrahydropyrimidin-1(2H)-yl)-1,3- dioxoisoindolin-5-yl)-2,5- diazabicyclo[2.2.1]heptan-2- yl)methyl)cyclohexyl)-1H-pyrazol-4- yl)pyrazolo[1,5-a]pyrimidine-3-carboxamide |
| GT- 0523 8 | 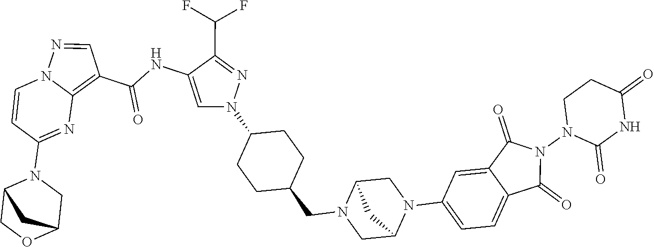 [SMILES: O=C1CCN(N2C(=O)c3ccc(N4C[C@H]5C[C@@H]4CN5C[C@H]4CC[C@H](n5cc(NC(=O)c6cnn7ccc(N8C[C@H]9C[C@@H]8CO9)nc67)c(C(F)F)n5)CC4)cc3C2=O)C(=O)N1] | 5-((1R,4R)-2-oxa-5-azabicyclo[2.2.1]heptan- 5-yl)-N-(3-(difluoromethyl)-1-((1R,4R)-4- (((1R,4R)-5-(2-(2,4- dioxotetrahydropyrimidin-1(2H)-yl)-1,3- dioxoisoindolin-5-yl)-2,5- diazabicyclo[2.2.1]heptan-2- yl)methyl)cyclohexyl)-1H-pyrazol-4- yl)pyrazolo[1,5-a]pyrimidine-3-carboxamide |
| GT- 0512 2 | 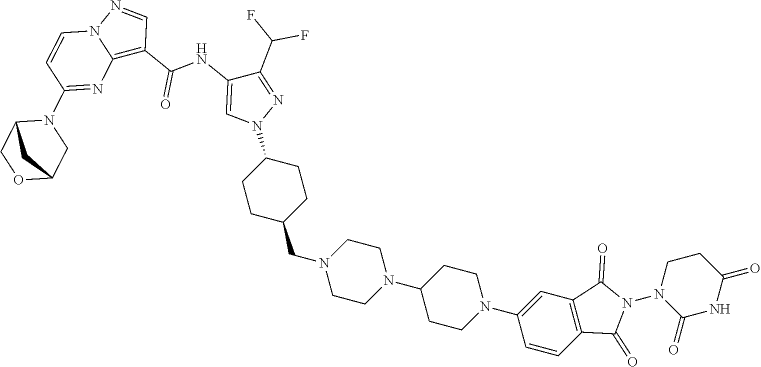 [SMILES: O=C1CCN(N2C(=O)c3ccc(N4CCC(N5CCN(C[C@H]6CC[C@H](n7cc(NC(=O)c8cnn9ccc(N%10C[C@H]%11C[C@@H]%10CO%11)nc89)c(C(F)F)n7)CC6)CC5)CC4)cc3C2=O)C(=O)N1] | 5-((1R,4R)-2-oxa-5-azabicyclo[2.2.1]heptan- 5-yl)-N-(3-(difluoromethyl)-1-((1r,4R)-4-((4- (1-(2-(2,4-dioxotetrahydropyrimidin-1(2H)- yl)-1,3-dioxoisoindolin-5-yl)piperidin-4- yl)piperazin-1-yl)methyl)cyclohexyl)-1H- pyrazol-4-yl)pyrazolo[1,5-a]pyrimidine-3- carboxamide |
| GT- 0523 9 | 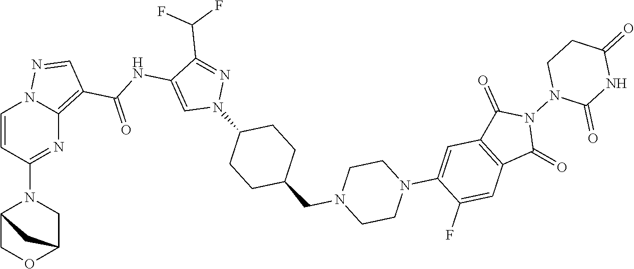 [SMILES: O=C1CCN(N2C(=O)c3cc(F)c(N4CCN(C[C@H]5CC[C@H](n6cc(NC(=O)c7cnn8ccc(N9C[C@H]%10C[C@@H]9CO%10)nc78)c(C(F)F)n6)CC5)CC4)cc3C2=O)C(=O)N1] | 5-((1R,4R)-2-oxa-5-azabicyclo[2.2.1]heptan- 5-yl)-N-(3-(difluoromethyl)-1-((1r,4R)-4-((4- (2-(2,4-dioxotetrahydropyrimidin-1(2H)-yl)- 6-fluoro-1,3-dioxoisoindolin-5-yl)piperazin- 1-yl)methyl)cyclohexyl)-1H-pyrazol-4- yl)pyrazolo[1,5-a]pyrimidine-3-carboxamide |
| GT- 0524 0 | 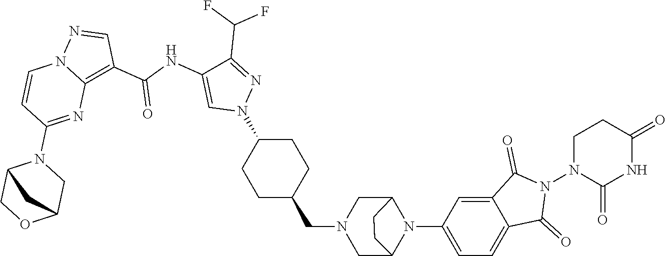 [SMILES: O=C1CCN(N2C(=O)c3ccc(N4C5CCC4CN(C[C@H]4CC[C@H](n6cc(NC(=O)c7cnn8ccc(N9C[C@H]%10C[C@@H]9CO%10)nc78)c(C(F)F)n6)CC4)C5)cc3C2=O)C(=O)N1] | 5-((1R,4R)-2-oxa-5-azabicyclo[2.2.1]heptan- 5-yl)-N-(3-(difluoromethyl)-1-((1r,4R)-4-((8- (2-(2,4-dioxotetrahydropyrimidin-1(2H)-yl)- 1,3-dioxoisoindolin-5-yl)-3,8- diazabicyclo[3.2.1]octan-3- yl)methyl)cyclohexyl)-1H-pyrazol-4- yl)pyrazolo[1,5-a]pyrimidine-3-carboxamide |
| GT- 0524 1 | 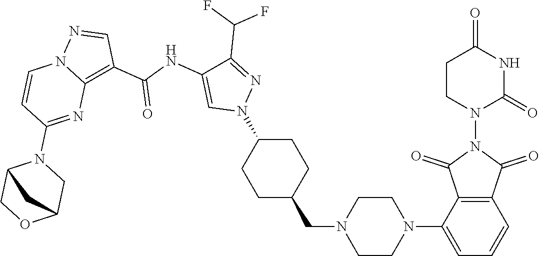 [SMILES: O=C1CCN(N2C(=O)c3cccc(N4CCN(C[C@H]5CC[C@H](n6cc(NC(=O)c7cnn8ccc(N9C[C@H]%10C[C@@H]9CO%10)nc78)c(C(F)F)n6)CC5)CC4)c3C2=O)C(=O)N1] | 5-((1R,4R)-2-oxa-5-azabicyclo[2.2.1]heptan- 5-yl)-N-(3-(difluoromethyl)-1-((1r,4R)-4-((4- (2-(2,4-dioxotetrahydropyrimidin-1(2H)-yl)- 1,3-dioxoisoindolin-4-yl)piperazin-1- yl)methyl)cyclohexyl)-1H-pyrazol-4- yl)pyrazolo[1,5-a]pyrimidine-3-carboxamide |
| GT- 0524 2 | 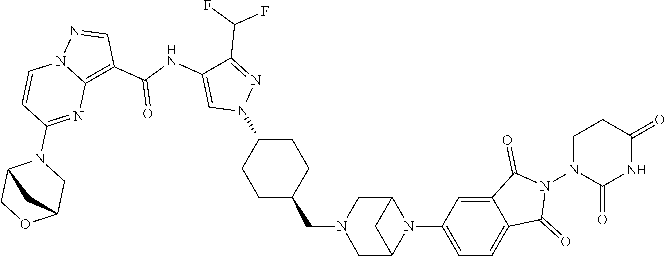 [SMILES: O=C1CCN(N2C(=O)c3ccc(N4C5CC4CN(C[C@H]4CC[C@H](n6cc(NC(=O)c7cnn8ccc(N9C[C@H]%10C[C@@H]9CO%10)nc78)c(C(F)F)n6)CC4)C5)cc3C2=O)C(=O)N1] | 5-((1R,4R)-2-oxa-5-azabicyclo[2.2.1]heptan- 5-yl)-N-(3-(difluoromethyl)-1-((1r,4R)-4-((6- (2-(2,4-dioxotetrahydropyrimidin-1(2H)-yl)- 1,3-dioxoisoindolin-5-yl)-3,6- diazabicyclo[3.1.1]heptan-3- yl)methyl)cyclohexyl)-1H-pyrazol-4- yl)pyrazolo[1,5-a]pyrimidine-3-carboxamide |
| GT- 0529 9 | 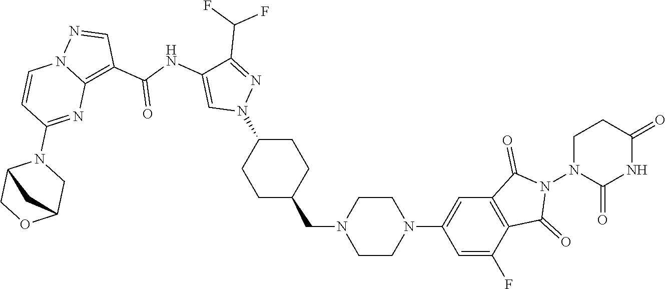 [SMILES: O=C1CCN(N2C(=O)c3cc(N4CCN(C[C@H]5CC[C@H](n6cc(NC(=O)c7cnn8ccc(N9C[C@H]%10C[C@@H]9CO%10)nc78)c(C(F)F)n6)CC5)CC4)cc(F)c3C2=O)C(=O)N1] | 5-((1R,4R)-2-oxa-5-azabicyclo[2.2.1]heptan- 5-yl)-N-(3-(difluoromethyl)-1-((1r,4R)-4-((4- (2-(2,4-dioxotetrahydropyrimidin-1(2H)-yl)- 7-fluoro-1,3-dioxoisoindolin-5-yl)piperazin- 1-yl)methyl)cyclohexyl)-1H-pyrazol-4- yl)pyrazolo[1,5-a]pyrimidine-3-carboxamide |
| GT- 0512 3 | 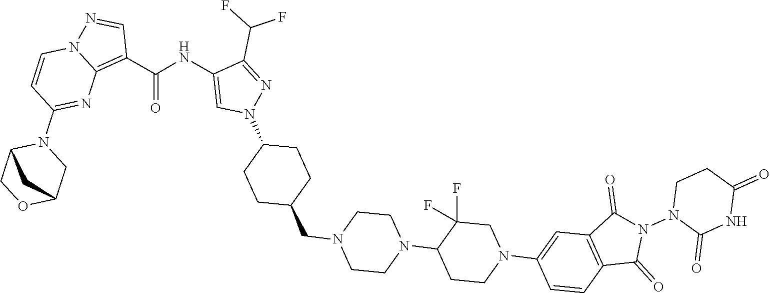 [SMILES: O=C1CCN(N2C(=O)c3ccc(N4CCC(N5CCN(C[C@H]6CC[C@H](n7cc(NC(=O)c8cnn9ccc(N%10C[C@H]%11C[C@@H]%10CO%11)nc89)c(C(F)F)n7)CC6)CC5)C(F)(F)C4)cc3C2=O)C(=O)N1] | 5-((1R,4R)-2-oxa-5-azabicyclo[2.2.1]heptan- 5-yl)-N-(3-(difluoromethyl)-1-((1r,4R)-4-((4- (1-(2-(2,4-dioxotetrahydropyrimidin-1(2H)- yl)-1,3-dioxoisoindolin-5-yl)-3,3- difluoropiperidin-4-yl)piperazin-1- yl)methyl)cyclohexyl)-1H-pyrazol-4- yl)pyrazolo[1,5-a]pyrimidine-3-carboxamide |
| | 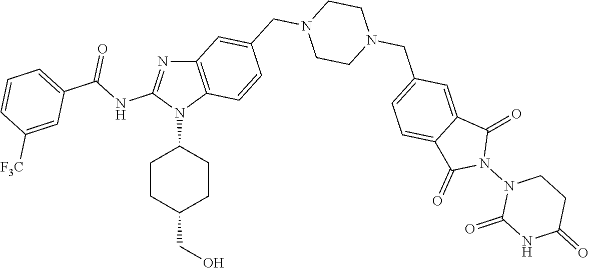 [SMILES: O=C1CCN(N2C(=O)c3ccc(CN4CCN(Cc5ccc6c(c5)nc(NC(=O)c5cccc(C(F)(F)F)c5)n6[C@H]5CC[C@@H](CO)CC5)CC4)cc3C2=O)C(=O)N1] | N-(5-((4-((2-(2,4-dioxotetrahydropyrimidin- 1(2H)-yl)-1,3-dioxoisoindolin-5- yl)methyl)piperazin-1-yl)methyl)-1-((1s,4s)- 4-(hydroxymethyl)cyclohexyl)-1H- benzo[d]imidazol-2-yl)-3- (trifluoromethyl)benzamide |
| GT- 0508 1 | 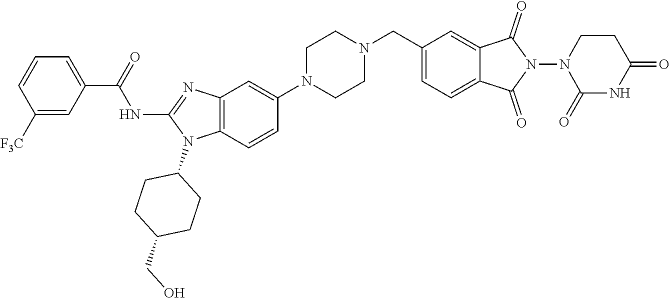 [SMILES: O=C1CCN(N2C(=O)c3ccc(CN4CCN(c5ccc6c(c5)nc(NC(=O)c5cccc(C(F)(F)F)c5)n6[C@H]5CC[C@@H](CO)CC5)CC4)cc3C2=O)C(=O)N1] | N-(5-(4-((2-(2,4-dioxotetrahydropyrimidin- 1(2H)-yl)-1,3-dioxoisoindolin-5- yl)methyl)piperazin-1-yl)-1-((1s,4s)-4- (hydroxymethyl)cyclohexyl)-1H- benzo[d]imidazol-2-yl)-3- (trifluoromethyl)benzamide |
| GT- 0505 5 | 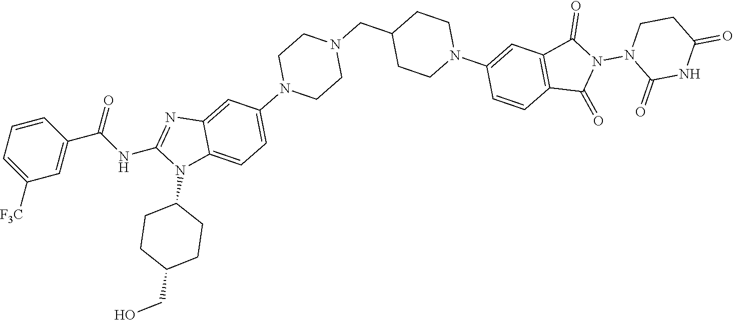 [SMILES: O=C1CCN(N2C(=O)c3ccc(N4CCC(CN5CCN(c6ccc7c(c6)nc(NC(=O)c6cccc(C(F)(F)F)c6)n7[C@H]6CC[C@@H](CO)CC6)CC5)CC4)cc3C2=O)C(=O)N1] | N-(5-(4-((1-(2-(2,4- dioxotetrahydropyrimidin-1(2H)-yl)-1,3- dioxoisoindolin-5-yl)piperidin-4- yl)methyl)piperazin-1-yl)-1-((1s,4s)-4- (hydroxymethyl)cyclohexyl)-1H- benzo[d]imidazol-2-yl)-3- (trifluoromethyl)benzamide |
| GT- 0532 5 | 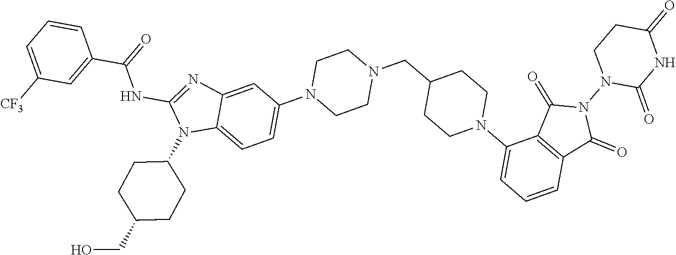 [SMILES: O=C1CCN(N2C(=O)c3cccc(N4CCC(CN5CCN(c6ccc7c(c6)nc(NC(=O)c6cccc(C(F)(F)F)c6)n7[C@H]6CC[C@@H](CO)CC6)CC5)CC4)c3C2=O)C(=O)N1] | N-(5-(4-((1-(2-(2,4- dioxotetrahydropyrimidin-1(2H)-yl)-1,3- dioxoisoindolin-4-yl)piperidin-4- yl)methyl)piperazin-1-yl)-1-((1s,4s)-4- (hydroxymethyl)cyclohexyl)-1H- benzo[d]imidazol-2-yl)-3- (trifluoromethyl)benzamide |
| | 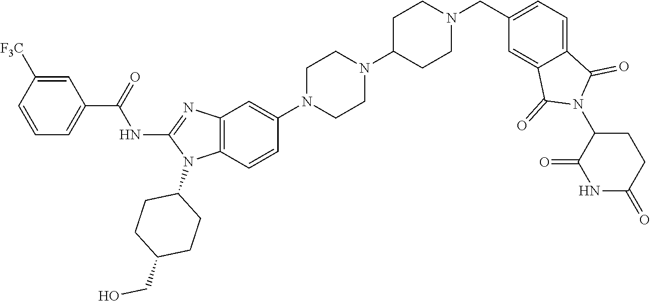 [SMILES: O=C1CCC(N2C(=O)c3ccc(CN4CCC(N5CCN(c6ccc7c(c6)nc(NC(=O)c6cccc(C(F)(F)F)c6)n7[C@H]6CC[C@@H](CO)CC6)CC5)CC4)cc3C2=O)C(=O)N1] | N-(5-(4-(1-((2-(2,4- dioxotetrahydropyrimidin-1(2H)-yl)-1,3- dioxoisoindolin-5-yl)methyl)piperidin-4- yl)piperazin-1-yl)-1-((1s,4s)-4- (hydroxymethyl)cyclohexyl)-1H- benzo[d]imidazol-2-yl)-3- (trifluoromethyl)benzamide |
| GT- 0532 6 | 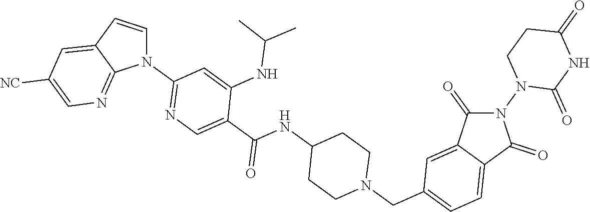 [SMILES: CC(C)Nc1cc(-n2ccc3cc(C#N)cnc32)ncc1C(=O)NC1CCN(Cc2ccc3c(c2)C(=O)N(N2CCC(=O)NC2=O)C3=O)CC1] | 6-(5-cyano-1H-pyrrolo[2,3-b]pyridin-1-yl)-N- (1-((2-(2,4-dioxotetrahydropyrimidin-1(2H)- yl)-1,3-dioxoisoindolin-5- yl)methyl)piperidin-4-yl)-4- (isopropylamino)nicotinamide |
| GT- 0518 8 | 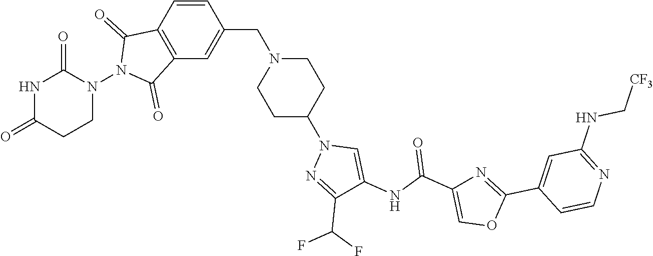 [SMILES: O=C1CCN(N2C(=O)c3ccc(CN4CCC(n5cc(NC(=O)c6coc(-c7ccnc(NCC(F)(F)F)c7)n6)c(C(F)F)n5)CC4)cc3C2=O)C(=O)N1] | N-(3-(difluoromethyl)-1-(1-((2-(2,4- dioxotetrahydropyrimidin-1(2H)-yl)-1,3- dioxoisoindolin-5-yl)methyl)piperidin-4-yl)- 1H-pyrazol-4-yl)-2-(2-((2,2,2- trifluoroethyl)amino)pyridin-4-yl)oxazole-4- carboxamide |
| GT- 0514 2 | 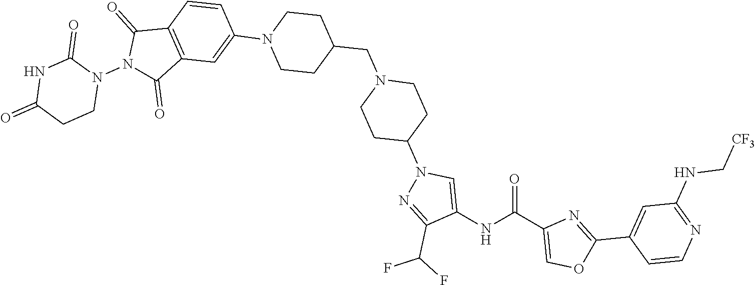 [SMILES: O=C1CCN(N2C(=O)c3ccc(N4CCC(CN5CCC(n6cc(NC(=O)c7coc(-c8ccnc(NCC(F)(F)F)c8)n7)c(C(F)F)n6)CC5)CC4)cc3C2=O)C(=O)N1] | N-(3-(difluoromethyl)-1-(1-((1-(2-(2,4- dioxotetrahydropyrimidin-1(2H)-yl)-1,3- dioxoisoindolin-5-yl)piperidin-4- yl)methyl)piperidin-4-yl)-1H-pyrazol-4-yl)-2- (2-((2,2,2-trifluoroethyl)amino)pyridin-4- yl)oxazole-4-carboxamide |
| GT- 0514 3 | 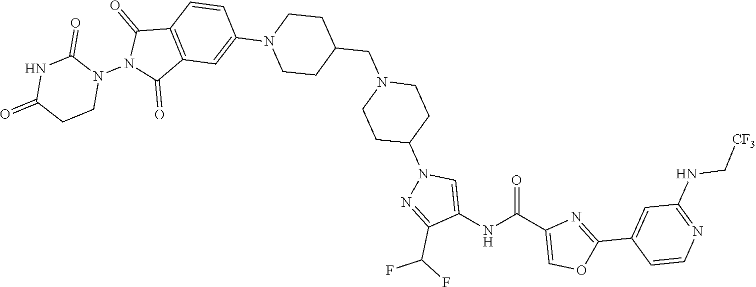 [SMILES: O=C1CCN(N2C(=O)c3ccc(N4CCC(CN5CCC(n6cc(NC(=O)c7coc(-c8ccnc(NCC(F)(F)F)c8)n7)c(C(F)F)n6)CC5)CC4)cc3C2=O)C(=O)N1] | N-(3-(difluoromethyl)-1-(1-((1-(2-(2,4- dioxotetrahydropyrimidin-1(2H)-yl)-1,3- dioxoisoindolin-5-yl)piperidin-4- yl)methyl)piperidin-4-yl)-1H-pyrazol-4-yl)-2- (2-((2,2,2-trifluoroethyl)amino)pyridin-4- yl)oxazole-4-carboxamide |
| GT- 0514 4 | 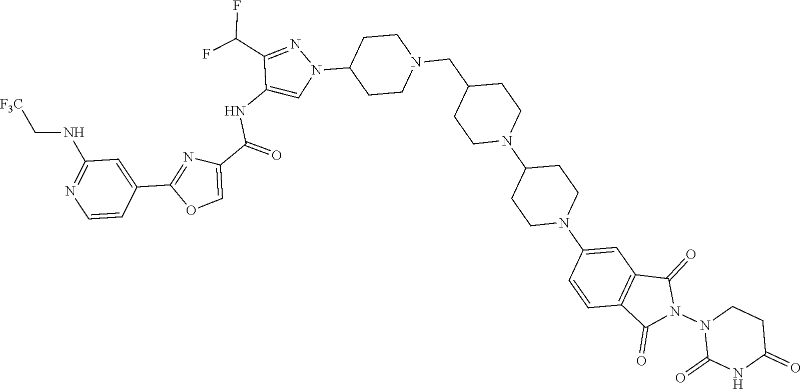 [SMILES: O=C1CCN(N2C(=O)c3ccc(N4CCC(N5CCC(CN6CCC(n7cc(NC(=O)c8coc(-c9ccnc(NCC(F)(F)F)c9)n8)c(C(F)F)n7)CC6)CC5)CC4)cc3C2=O)C(=O)N1] | N-(3-(difluoromethyl)-1-(1-((1′-(2-(2,4- dioxotetrahydropyrimidin-1(2H)-yl)-1,3- dioxoisoindolin-5-yl)-[1,4′-bipiperidin]-4- yl)methyl)piperidin-4-yl)-1H-pyrazol-4-yl)-2- (2-((2,2,2-trifluoroethyl)amino)pyridin-4- yl)oxazole-4-carboxamide |
| GT- 0580 3 | 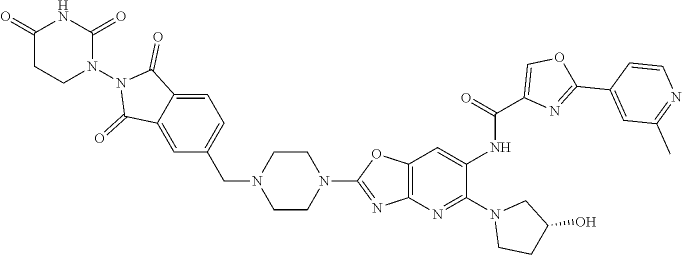 [SMILES: Cc1cc(-c2nc(C(=O)Nc3cc4oc(N5CCN(Cc6ccc7c(c6)C(=O)N(N6CCC(=O)NC6=O)C7=O)CC5)nc4nc3N3CC[C@@H](O)C3)co2)ccn1] | (R)-N-(2-(4-((2-(2,4- dioxotetrahydropyrimidin-1(2H)-yl)-1,3- dioxoisoindolin-5-yl)methyl)piperazin-1-yl)- 5-(3-hydroxypyrrolidin-1-yl)oxazolo[4,5- b]pyridin-6-yl)-2-(2-methylpyridin-4- yl)oxazole-4-carboxamide |
| GT- 0576 2 | 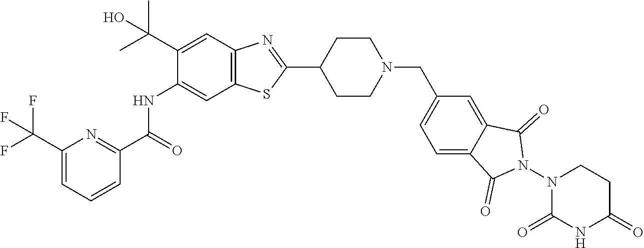 [SMILES: CC(C)(O)c1cc2nc(C3CCN(Cc4ccc5c(c4)C(=O)N(N4CCC(=O)NC4=O)C5=O)CC3)sc2cc1NC(=O)c1cccc(C(F)(F)F)n1] | N-(2-(1-((2-(2,4-dioxotetrahydropyrimidin- 1(2H)-yl)-1,3-dioxoisoindolin-5- yl)methyl)piperidin-4-yl)-5-(2- hydroxypropan-2-yl)benzo[d]thiazol-6-yl)-6- (trifluoromethyl)picolinamide |
| GT- 0577 0 | 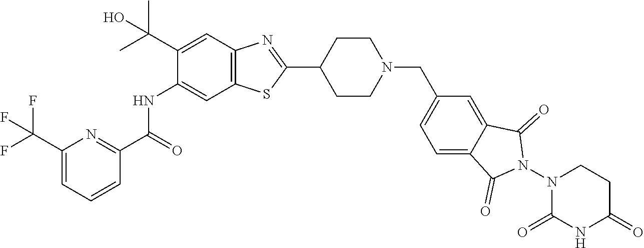 [SMILES: CC(C)(O)c1cc2nc(C3CCN(Cc4ccc5c(c4)C(=O)N(N4CCC(=O)NC4=O)C5=O)CC3)sc2cc1NC(=O)c1cccc(C(F)(F)F)n1] | N-(2-(1-((2-(2,4-dioxotetrahydropyrimidin- 1(2H)-yl)-1,3-dioxoisoindolin-5- yl)methyl)piperidin-4-yl)-5-(2- hydroxypropan-2-yl)benzo[d]oxazol-6-yl)-6- (trifluoromethyl)picolinamide |
| GT- 0522 2 | 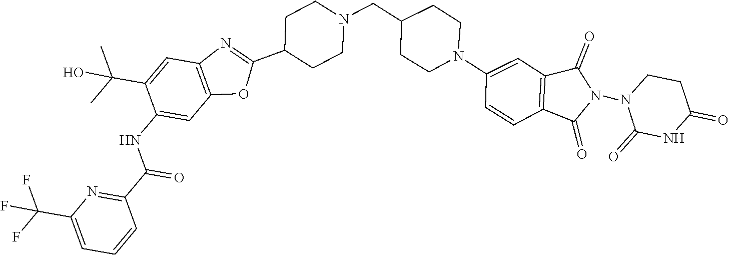 [SMILES: CC(C)(O)c1cc2nc(C3CCN(CC4CCN(c5ccc6c(c5)C(=O)N(N5CCC(=O)NC5=O)C6=O)CC4)CC3)oc2cc1NC(=O)c1cccc(C(F)(F)F)n1] | N-(2-(1-((1-(2-(2,4- dioxotetrahydropyrimidin-1(2H)-yl)-1,3- dioxoisoindolin-5-yl)piperidin-4- yl)methyl)piperidin-4-yl)-5-(2- hydroxypropan-2-yl)benzo[d]oxazol-6-yl)-6- (trifluoromethyl)picolinamide |
| GT- 0522 3 | 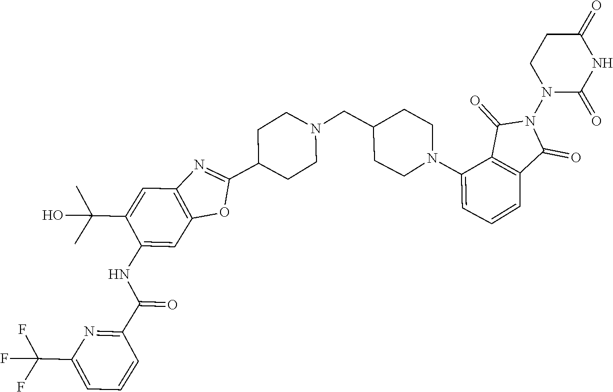 [SMILES: CC(C)(O)c1cc2nc(C3CCN(CC4CCN(c5cccc6c5C(=O)N(N5CCC(=O)NC5=O)C6=O)CC4)CC3)oc2cc1NC(=O)c1cccc(C(F)(F)F)n1] | N-(2-(1-((1-(2-(2,4- dioxotetrahydropyrimidin-1(2H)-yl)-1,3- dioxoisoindolin-4-yl)piperidin-4- yl)methyl)piperidin-4-yl)-5-(2- hydroxypropan-2-yl)benzo[d]oxazol-6-yl)-6- (trifluoromethyl)picolinamide |
| GT- 0575 2 | 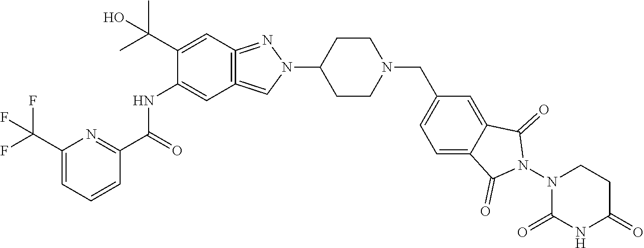 | N-(2-(1-((2-(2,4-dioxotetrahydropyrimidin- 1(2H)-yl)-1,3-dioxoisoindolin-5- yl)methyl)piperidin-4-yl)-6-(2- hydroxypropan-2-yl)-2H-indazol-5-yl)-6- (trifluoromethyl)picolinamide |
| GT- 0522 0 | 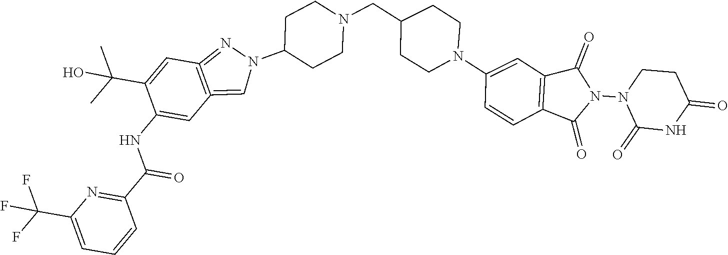 | N-(2-(1-((1-(2-(2,4- dioxotetrahydropyrimidin-1(2H)-yl)-1,3- dioxoisoindolin-5-yl)piperidin-4- yl)methyl)piperidin-4-yl)-6-(2- hydroxypropan-2-yl)-2H-indazol-5-yl)-6- (trifluoromethyl)picolinamide |
| GT- 0522 1 | 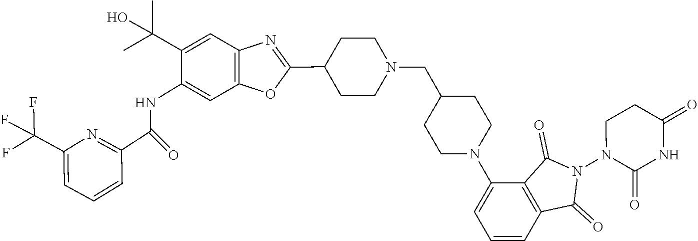 [SMILES: CC(C)(O)c1cc2nc(C3CCN(CC4CCN(c5cccc6c5C(=O)N(N5CCC(=O)NC5=O)C6=O)CC4)CC3)oc2cc1NC(=O)c1cccc(C(F)(F)F)n1] | N-(2-(1-((1-(2-(2,4- dioxotetrahydropyrimidin-1(2H)-yl)-1,3- dioxoisoindolin-4-yl)piperidin-4- yl)methyl)piperidin-4-yl)-6-(2- hydroxypropan-2-yl)-2H-indazol-5-yl)-6- (trifluoromethyl)picolinamide |
| GT- 0529 0 | 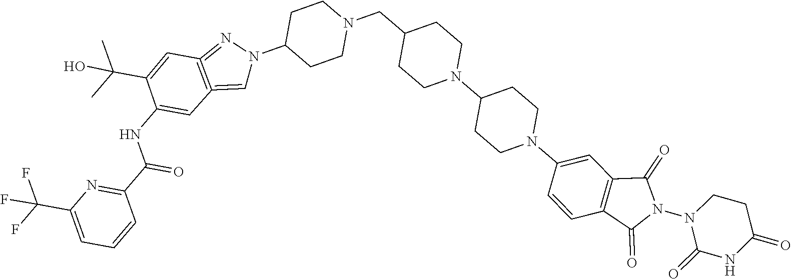 | N-(2-(1-((1′-(2-(2,4- dioxotetrahydropyrimidin-1(2H)-yl)-1,3- dioxoisoindolin-5-yl)-[1,4′-bipiperidin]-4- yl)methyl)piperidin-4-yl)-6-(2- hydroxypropan-2-yl)-2H-indazol-5-yl)-6- (trifluoromethyl)picolinamide |
| GT- 0841 9 | 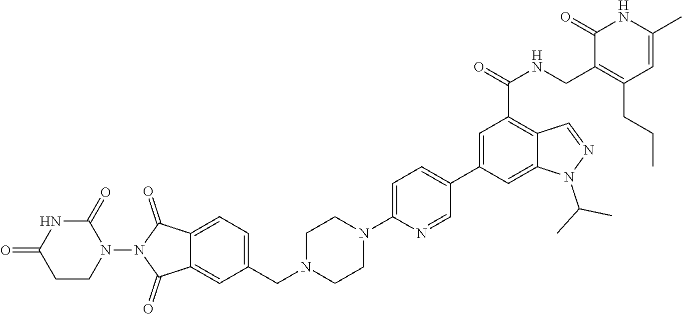 [SMILES: CCCc1cc(C)[nH]c(=O)c1CNC(=O)c1cc(-c2ccc(N3CCN(Cc4ccc5c(c4)C(=O)N(N4CCC(=O)NC4=O)C5=O)CC3)nc2)cc2c1cnn2C(C)C] | 6-(6-(4-((2-(2,4-dioxotetrahydropyrimidin- 1(2H)-yl)-1,3-dioxoisoindolin-5- yl)methyl)piperazin-1-yl)pyridin-3-yl)-1- isopropyl-N-((6-methyl-2-oxo-4-propyl-1,2- dihydropyridin-3-yl)methyl)-1H-indazole-4- carboxamide |
| GT- 0842 1 | 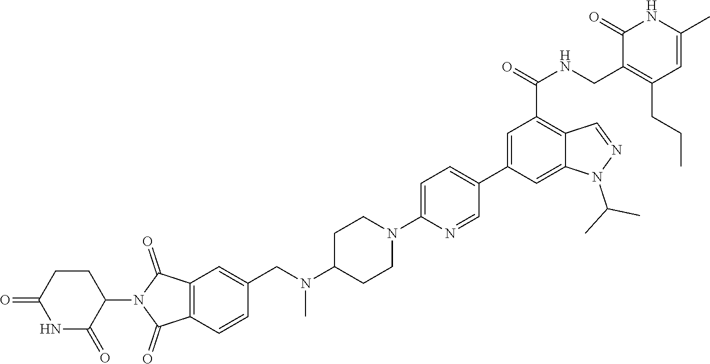 [SMILES: CCCc1cc(C)[nH]c(=O)c1CNC(=O)c1cc(-c2ccc(N3CCC(N(C)Cc4ccc5c(c4)C(=O)N(C4CCC(=O)NC4=O)C5=O)CC3)nc2)cc2c1cnn2C(C)C] | 6-(6-(4-(((2-(2,4-dioxotetrahydropyrimidin- 1(2H)-yl)-1,3-dioxoisoindolin-5- yl)methyl)(methyl)amino)piperidin-1- yl)pyridin-3-yl)-1-isopropyl-N-((6-methyl-2- oxo-4-propyl-1,2-dihydropyridin-3- yl)methyl)-1H-indazole-4-carboxamide |
| GT- 0842 3 | 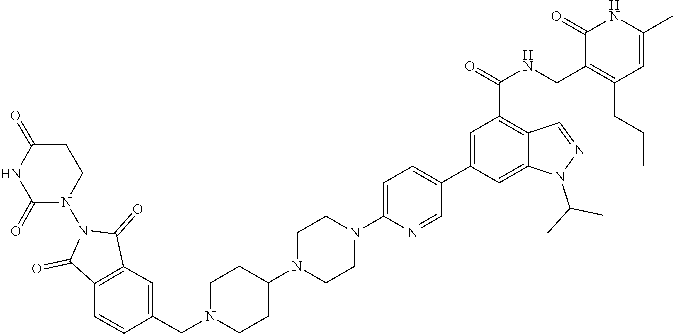 [SMILES: CCCc1cc(C)[nH]c(=O)c1CNC(=O)c1cc(-c2ccc(N3CCN(C4CCN(Cc5ccc6c(c5)C(=O)N(N5CCC(=O)NC5=O)C6=O)CC4)CC3)nc2)cc2c1cnn2C(C)C] | 6-(6-(4-(1-((2-(2,4-dioxotetrahydropyrimidin- 1(2H)-yl)-1,3-dioxoisoindolin-5- yl)methyl)piperidin-4-yl)piperazin-1- yl)pyridin-3-yl)-1-isopropyl-N-((6-methyl-2- oxo-4-propyl-1,2-dihydropyridin-3- yl)methyl)-1H-indazole-4-carboxamide |
| GT- 0850 9 | 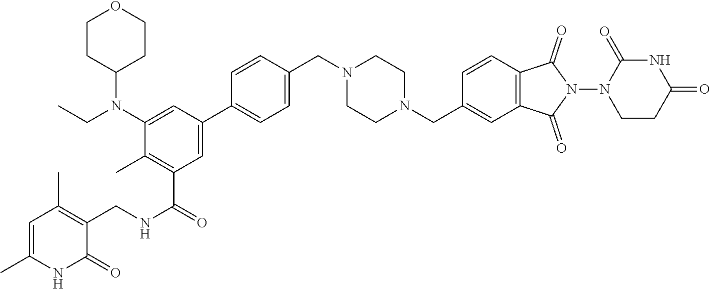 [SMILES: CCN(c1cc(-c2ccc(CN3CCN(Cc4ccc5c(c4)C(=O)N(N4CCC(=O)NC4=O)C5=O)CC3)cc2)cc(C(=O)NCc2c(C)cc(C)[nH]c2=O)c1C)C1CCOCC1] | N-((4,6-dimethyl-2-oxo-1,2-dihydropyridin- 3-yl)methyl)-4′-((4-((2-(2,4- dioxotetrahydropyrimidin-1(2H)-yl)-1,3- dioxoisoindolin-5-yl)methyl)piperazin-1- yl)methyl)-5-(ethyl(tetrahydro-2H-pyran-4- yl)amino)-4-methyl-[1,1′-biphenyl]-3- carboxamide |
| GT- 0851 1 | 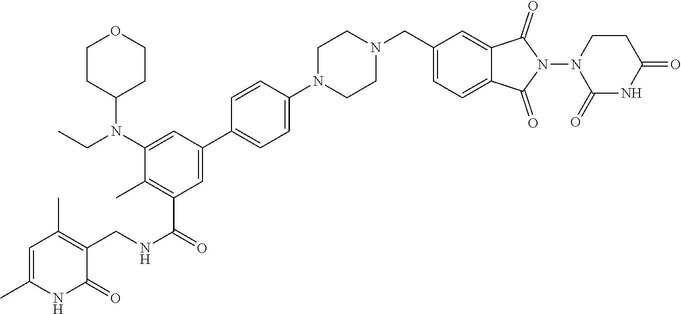 [SMILES: CCN(c1cc(-c2ccc(N3CCN(Cc4ccc5c(c4)C(=O)N(N4CCC(=O)NC4=O)C5=O)CC3)cc2)cc(C(=O)NCc2c(C)cc(C)[nH]c2=O)c1C)C1CCOCC1] | N-((4,6-dimethyl-2-oxo-1,2-dihydropyridin- 3-yl)methyl)-4′-(4-((2-(2,4- dioxotetrahydropyrimidin-1(2H)-yl)-1,3- dioxoisoindolin-5-yl)methyl)piperazin-1-yl)- 5-(ethyl(tetrahydro-2H-pyran-4-yl)amino)-4- methyl-[1,1′-biphenyl]-3-carboxamide |
| GT- 0851 3 | 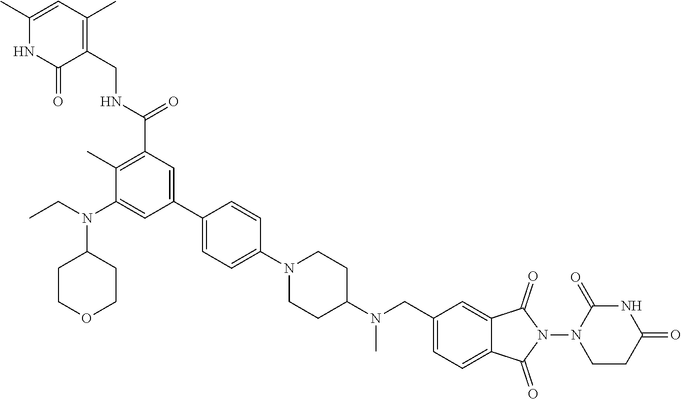 [SMILES: CCN(c1cc(-c2ccc(N3CCC(N(C)Cc4ccc5c(c4)C(=O)N(N4CCC(=O)NC4=O)C5=O)CC3)cc2)cc(C(=O)NCc2c(C)cc(C)[nH]c2=O)c1C)C1CCOCC1] | N-((4,6-dimethyl-2-oxo-1,2-dihydropyridin- 3-yl)methyl)-4′-(4-(((2-(2,4- dioxotetrahydropyrimidin-1(2H)-yl)-1,3- dioxoisoindolin-5- yl)methyl)(methyl)amino)piperidin-1-yl)-5- (ethyl(tetrahydro-2H-pyran-4-yl)amino)-4- methyl-[1,1′-biphenyl]-3-carboxamide |
| GT- 0851 5 | 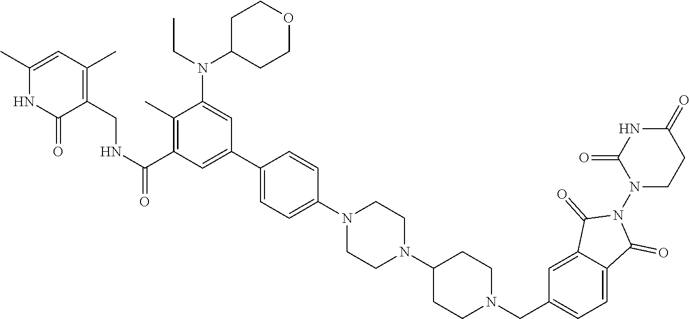 [SMILES: CCN(c1cc(-c2ccc(N3CCN(C4CCN(Cc5ccc6c(c5)C(=O)N(N5CCC(=O)NC5=O)C6=O)CC4)CC3)cc2)cc(C(=O)NCc2c(C)cc(C)[nH]c2=O)c1C)C1CCOCC1] | N-((4,6-dimethyl-2-oxo-1,2-dihydropyridin- 3-yl)methyl)-4′-(4-(1-((2-(2,4- dioxotetrahydropyrimidin-1(2H)-yl)-1,3- dioxoisoindolin-5-yl)methyl)piperidin-4- yl)piperazin-1-yl)-5-(ethyl(tetrahydro-2H- pyran-4-yl)amino)-4-methyl-[1,1′-biphenyl]- 3-carboxamide |
| | 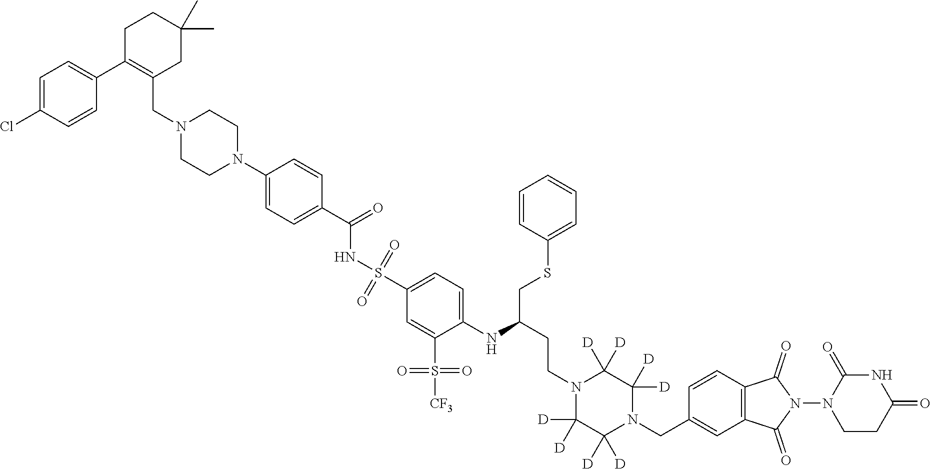 [SMILES: [2H]C1([2H])N(CC[C@H](CSc2ccccc2)Nc2ccc(S(=O)(=O)NC(=O)c3ccc(N4CCN(CC5=C(c6ccc(Cl)cc6)CCC(C)(C)C5)CC4)cc3)cc2S(=O)(=O)C(F)(F)F)C([2H])([2H])C([2H])([2H])N(Cc2ccc3c(c2)C(=O)N(N2CCC(=O)NC2=O)C3=O)C1([2H])[2H]] | (R)-4-(4-((4′-chloro-4,4-dimethyl-3,4,5,6- tetrahydro-[1,1′-biphenyl]-2- yl)methyl)piperazin-1-yl)-N-((4-((4-(4-((2- (2,4-dioxotetrahydropyrimidin-1(2H)-yl)-1,3- dioxoisoindolin-5-yl)methyl)piperazin-1-yl- 2,2,3,3,5,5,6,6-d₈)-1-(phenylthio)butan-2- yl)amino)-3- ((trifluoromethyl)sulfonyl)phenyl)sulfonyl) benzamide |
| | 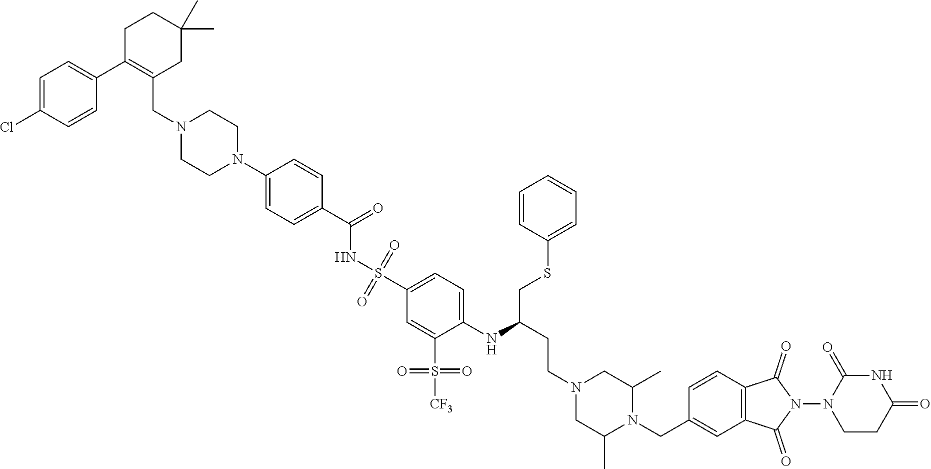 [SMILES: CC1CN(CC[C@H](CSc2ccccc2)Nc2ccc(S(=O)(=O)NC(=O)c3ccc(N4CCN(CC5=C(c6ccc(Cl)cc6)CCC(C)(C)C5)CC4)cc3)cc2S(=O)(=O)C(F)(F)F)CC(C)N1Cc1ccc2c(c1)C(=O)N(N1CCC(=O)NC1=O)C2=O] | 4-(4-((4′-chloro-4,4-dimethyl-3,4,5,6- tetrahydro-[1,1′-biphenyl]-2- yl)methyl)piperazin-1-yl)-N-((4-(((2R)-4-(4- ((2-(2,4-dioxotetrahydropyrimidin-1(2H)-yl)- 1,3-dioxoisoindolin-5-yl)methyl)-3,5- dimethylpiperazin-1-yl)-1-(phenylthio)butan- 2-yl)amino)-3- ((trifluoromethyl)sulfonyl)phenyl)sulfonyl) benzamide |
| | 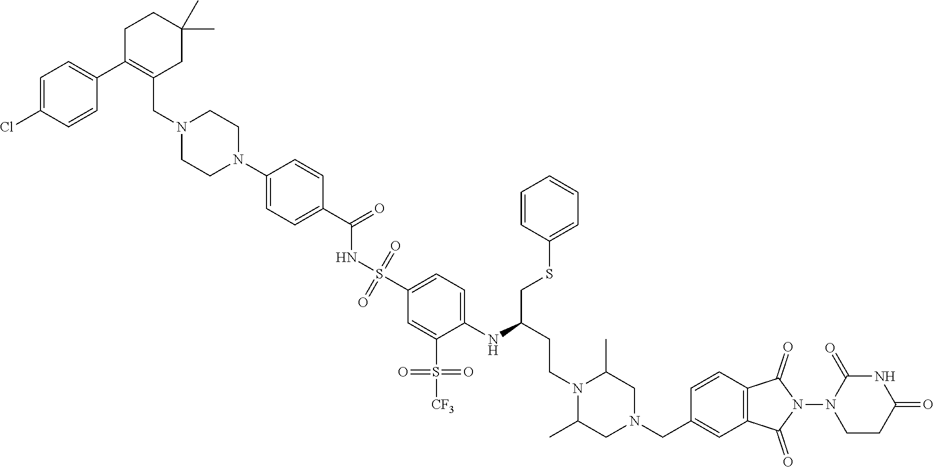 [SMILES: CC1CN(Cc2ccc3c(c2)C(=O)N(N2CCC(=O)NC2=O)C3=O)CC(C)N1CC[C@H](CSc1ccccc1)Nc1ccc(S(=O)(=O)NC(=O)c2ccc(N3CCN(CC4=C(c5ccc(Cl)cc5)CCC(C)(C)C4)CC3)cc2)cc1S(=O)(=O)C(F)(F)F] | 4-(4-((4′-chloro-4,4-dimethyl-3,4,5,6- tetrahydro-[1,1′-biphenyl]-2- yl)methyl)piperazin-1-yl)-N-((4-(((2R)-4-(4- ((2-(2,4-dioxotetrahydropyrimidin-1(2H)-yl)- 1,3-dioxoisoindolin-5-yl)methyl)-2,6- dimethylpiperazin-1-yl)-1-(phenylthio)butan- 2-yl)amino)-3- ((trifluoromethyl)sulfonyl)phenyl)sulfonyl) benzamide |
| | 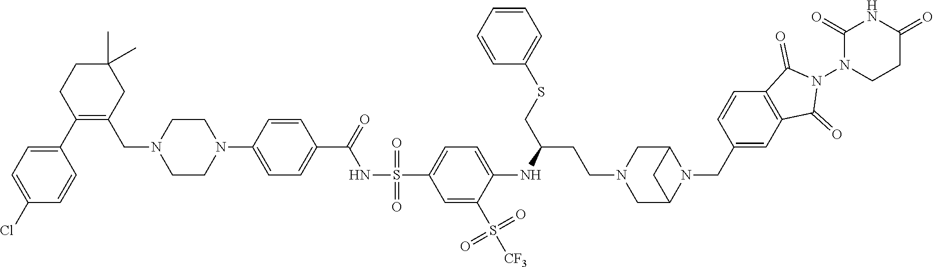 [SMILES: CC1(C)CCC(c2ccc(Cl)cc2)=C(CN2CCN(c3ccc(C(=O)NS(=O)(=O)c4ccc(N[C@H](CCN5CC6CC(C5)N6Cc5ccc6c(c5)C(=O)N(N5CCC(=O)NC5=O)C6=O)CSc5ccccc5)c(S(=O)(=O)C(F)(F)F)c4)cc3)CC2)C1] | 4-(4-((4′-chloro-4,4-dimethyl-3,4,5,6- tetrahydro-[1,1′-biphenyl]-2- yl)methyl)piperazin-1-yl)-N-((4-(((2R)-4-(6- ((2-(2,4-dioxotetrahydropyrimidin-1(2H)-yl)- 1,3-dioxoisoindolin-5-yl)methyl)-3,6- diazabicyclo[3.1.1]heptan-3-yl)-1- (phenylthio)butan-2-yl)amino)-3- ((trifluoromethyl)sulfonyl)phenyl)sulfonyl) benzamide |
| | 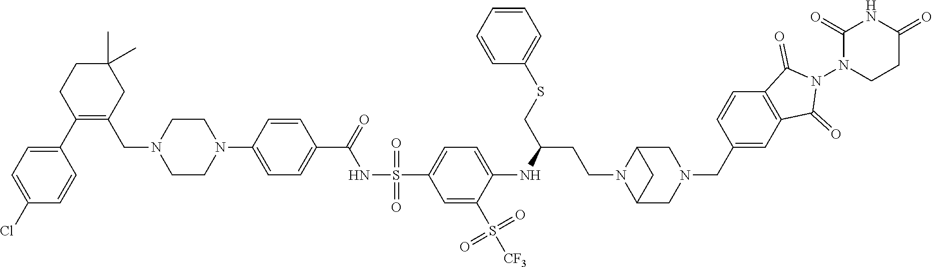 [SMILES: CC1(C)CCC(c2ccc(Cl)cc2)=C(CN2CCN(c3ccc(C(=O)NS(=O)(=O)c4ccc(N[C@H](CCN5C6CC5CN(Cc5ccc7c(c5)C(=O)N(N5CCC(=O)NC5=O)C7=O)C6)CSc5ccccc5)c(S(=O)(=O)C(F)(F)F)c4)cc3)CC2)C1] | 4-(4-((4′-chloro-4,4-dimethyl-3,4,5,6- tetrahydro-[1,1′-biphenyl]-2- yl)methyl)piperazin-1-yl)-N-((4-(((2R)-4-(3- ((2-(2,4-dioxotetrahydropyrimidin-1(2H)-yl)- 1,3-dioxoisoindolin-5-yl)methyl)-3,6- diazabicyclo[3.1.1]heptan-6-yl)-1- (phenylthio)butan-2-yl)amino)-3- ((trifluoromethyl)sulfonyl)phenyl)sulfonyl) benzamide |
| GT- 0781 7 | 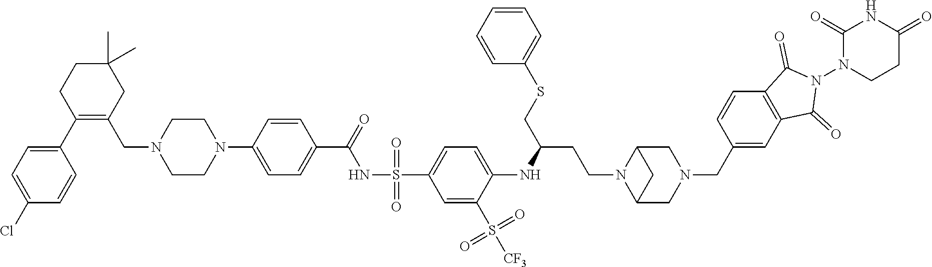 [SMILES: CC1(C)CCC(c2ccc(Cl)cc2)=C(CN2CCN(c3ccc(C(=O)NS(=O)(=O)c4ccc(N[C@H](CCN5C6CC5CN(Cc5ccc7c(c5)C(=O)N(N5CCC(=O)NC5=O)C7=O)C6)CSc5ccccc5)c(S(=O)(=O)C(F)(F)F)c4)cc3)CC2)C1] | 4-(4-((4′-chloro-4,4-dimethyl-3,4,5,6- tetrahydro-[1,1′-biphenyl]-2- yl)methyl)piperazin-1-yl)-N-((4-(((2R)-4-(8- ((2-(2,4-dioxotetrahydropyrimidin-1(2H)-yl)- 1,3-dioxoisoindolin-5-yl)methyl)-3,8- diazabicyclo[3.2.1]octan-3-yl)-1- (phenylthio)butan-2-yl)amino)-3- ((trifluoromethyl)sulfonyl)phenyl)sulfonyl) benzamide |
| GT- 0782 1 | 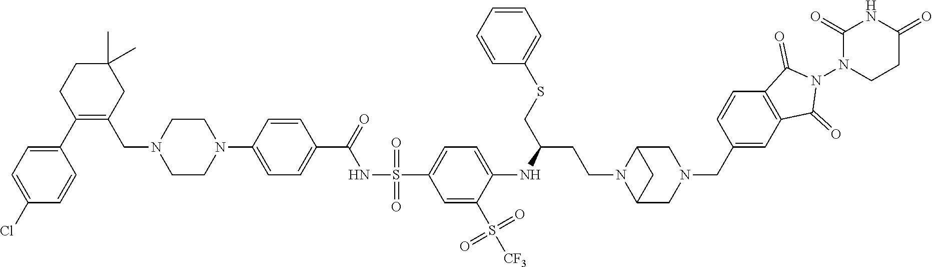 [SMILES: CC1(C)CCC(c2ccc(Cl)cc2)=C(CN2CCN(c3ccc(C(=O)NS(=O)(=O)c4ccc(N[C@H](CCN5C6CC5CN(Cc5ccc7c(c5)C(=O)N(N5CCC(=O)NC5=O)C7=O)C6)CSc5ccccc5)c(S(=O)(=O)C(F)(F)F)c4)cc3)CC2)C1] | 4-(4-((4′-chloro-4,4-dimethyl-3,4,5,6- tetrahydro-[1,1′-biphenyl]-2- yl)methyl)piperazin-1-yl)-N-((4-(((2R)-4-(3- ((2-(2,4-dioxotetrahydropyrimidin-1(2H)-yl)- 1,3-dioxoisoindolin-5-yl)methyl)-3,8- diazabicyclo[3.2.1]octan-8-yl)-1- (phenylthio)butan-2-yl)amino)-3- ((trifluoromethyl)sulfonyl)phenyl)sulfonyl) benzamide |
| GT- 0781 6 | 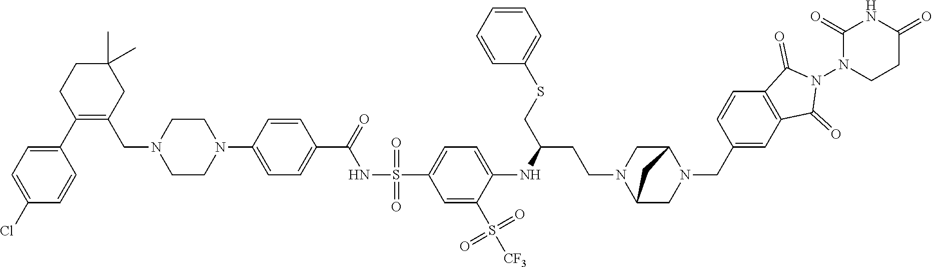 [SMILES: CC1(C)CCC(c2ccc(Cl)cc2)=C(CN2CCN(c3ccc(C(=O)NS(=O)(=O)c4ccc(N[C@H](CCN5C[C@H]6C[C@@H]5CN6Cc5ccc6c(c5)C(=O)N(N5CCC(=O)NC5=O)C6=O)CSc5ccccc5)c(S(=O)(=O)C(F)(F)F)c4)cc3)CC2)C1] | 4-(4-((4′-chloro-4,4-dimethyl-3,4,5,6- tetrahydro-[1,1′-biphenyl]-2- yl)methyl)piperazin-1-yl)-N-((4-(((R)-4- ((1R,4R)-5-((2-(2,4- dioxotetrahydropyrimidin-1(2H)-yl)-1,3- dioxoisoindolin-5-yl)methyl)-2,5- diazabicyclo[2.2.1]heptan-2-yl)-1- (phenylthio)butan-2-yl)amino)-3- ((trifluoromethyl)sulfonyl)phenyl)sulfonyl) benzamide |
| | 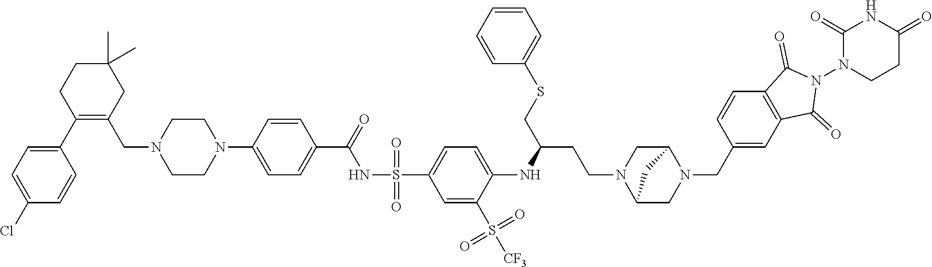 [SMILES: CC1(C)CCC(c2ccc(Cl)cc2)=C(CN2CCN(c3ccc(C(=O)NS(=O)(=O)c4ccc(N[C@H](CCN5C[C@@H]6C[C@H]5CN6Cc5ccc6c(c5)C(=O)N(N5CCC(=O)NC5=O)C6=O)CSc5ccccc5)c(S(=O)(=O)C(F)(F)F)c4)cc3)CC2)C1] | 4-(4-((4′-chloro-4,4-dimethyl-3,4,5,6- tetrahydro-[1,1′-biphenyl]-2- yl)methyl)piperazin-1-yl)-N-((4-(((R)-4- ((1S,4S)-5-((2-(2,4- dioxotetrahydropyrimidin-1(2H)-yl)-1,3- dioxoisoindolin-5-yl)methyl)-2,5- diazabicyclo[2.2.1]heptan-2-yl)-1- (phenylthio)butan-2-yl)amino)-3- ((trifluoromethyl)sulfonyl)phenyl)sulfonyl) benzamide |
| GT- 0781 5 | 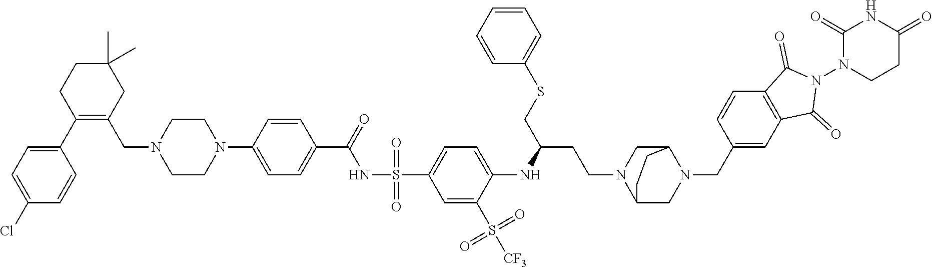 [SMILES: CC1(C)CCC(c2ccc(Cl)cc2)=C(CN2CCN(c3ccc(C(=O)NS(=O)(=O)c4ccc(N[C@H](CCN5CC6CCC5CN6Cc5ccc6c(c5)C(=O)N(N5CCC(=O)NC5=O)C6=O)CSc5ccccc5)c(S(=O)(=O)C(F)(F)F)c4)cc3)CC2)C1] | 4-(4-((4′-chloro-4,4-dimethyl-3,4,5,6- tetrahydro-[1,1′-biphenyl]-2- yl)methyl)piperazin-1-yl)-N-((4-(((2R)-4-(5- ((2-(2,4-dioxotetrahydropyrimidin-1(2H)-yl)- 1,3-dioxoisoindolin-5-yl)methyl)-2,5- diazabicyclo[2.2.2]octan-2-yl)-1- (phenylthio)butan-2-yl)amino)-3- ((trifluoromethyl)sulfonyl)phenyl)sulfonyl) benzamide |
| | 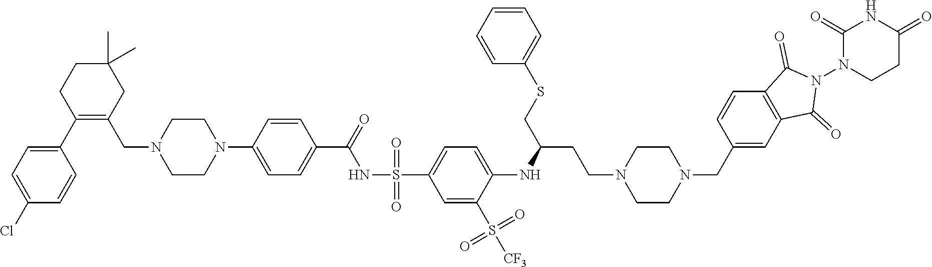 [SMILES: CC1(C)CCC(c2ccc(Cl)cc2)=C(CN2CCN(c3ccc(C(=O)NS(=O)(=O)c4ccc(N[C@H](CCN5CCN(Cc6ccc7c(c6)C(=O)N(N6CCC(=O)NC6=O)C7=O)CC5)CSc5ccccc5)c(S(=O)(=O)C(F)(F)F)c4)cc3)CC2)C1] | (R)-4-(4-((4′-chloro-4,4-dimethyl-3,4,5,6- tetrahydro-[1,1′-biphenyl]-2- yl)methyl)piperazin-1-yl)-N-((4-((4-(4-((2- (2,4-dioxotetrahydropyrimidin-1(2H)-yl)-1,3- dioxoisoindolin-5-yl)methyl)piperazin-1-yl)- 1-(phenylthio)butan-2-yl)amino)-3- ((trifluoromethyl)sulfonyl)phenyl)sulfonyl) benzamide |
| | 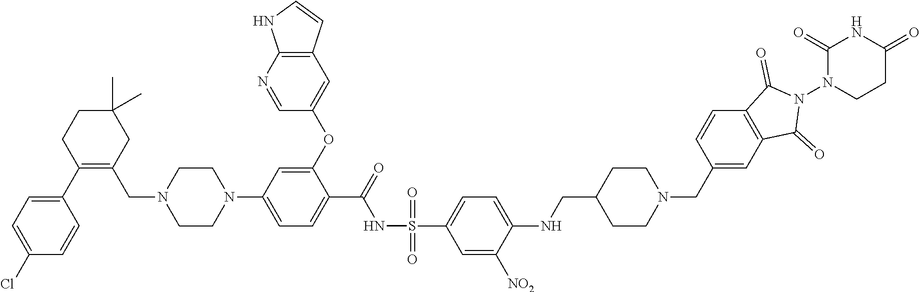 [SMILES: CC1(C)CCC(c2ccc(Cl)cc2)=C(CN2CCN(c3ccc(C(=O)NS(=O)(=O)c4ccc(NCC5CCN(Cc6ccc7c(c6)C(=O)N(N6CCC(=O)NC6=O)C7=O)CC5)c([N+](=O)[O-])c4)c(Oc4cnc5[nH]ccc5c4)c3)CC2)C1] | 2-((1H-pyrrolo[2,3-b]pyridin-5-yl)oxy)-4-(4- ((4′-chloro-5,5-dimethyl-3,4,5,6-tetrahydro- [1,1′-biphenyl]-2-yl)methyl)piperazin-1-yl)- N-((4-(((1-((2-(2,4-dioxotetrahydropyrimidin- 1(2H)-yl)-1,3-dioxoisoindolin-5- yl)methyl)piperidin-4-yl)methyl)amino)-3- nitrophenyl)sulfonyl)benzamide |
| | 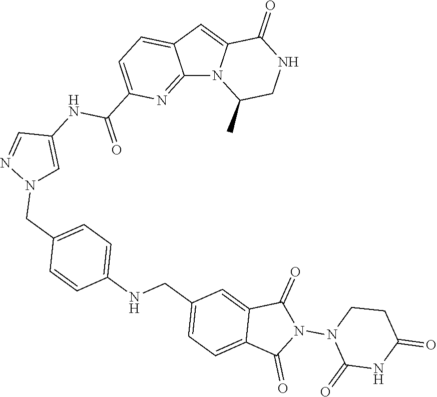 [SMILES: C[C@@H]1CNC(=O)c2cc3ccc(C(=O)Nc4cnn(Cc5ccc(NCc6ccc7c(c6)C(=O)N(N6CCC(=O)NC6=O)C7=O)cc5)c4)nc3n21] | (R)-N-(1-(4-(((2-(2,4- dioxotetrahydropyrimidin-1(2H)-yl)-1,3- dioxoisoindolin-5-yl)methyl)amino)benzyl)- 1H-pyrazol-4-yl)-9-methyl-6-oxo-6,7,8,9- tetrahydropyrido[3′,2′:4,5]pyrrolo[1,2- a]pyrazine-2-carboxamide |
| GT- 0815 0 | 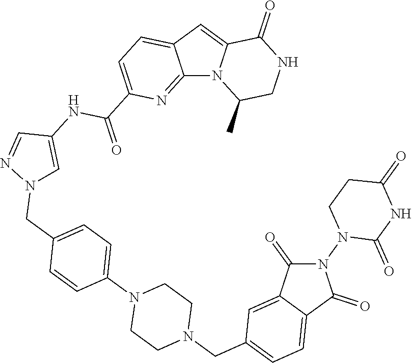 [SMILES: C[C@@H]1CNC(=O)c2cc3ccc(C(=O)Nc4cnn(Cc5ccc(N6CCN(Cc7ccc8c(c7)C(=O)N(N7CCC(=O)NC7=O)C8=O)CC6)cc5)c4)nc3n21] | (R)-N-(1-(4-(4-((2-(2,4- dioxotetrahydropyrimidin-1(2H)-yl)-1,3- dioxoisoindolin-5-yl)methyl)piperazin-1- yl)benzyl)-1H-pyrazol-4-yl)-9-methyl-6-oxo- 6,7,8,9-tetrahydropyrido[3′,2′:4,5]pyrrolo[1,2- a]pyrazine-2-carboxamide |
| | 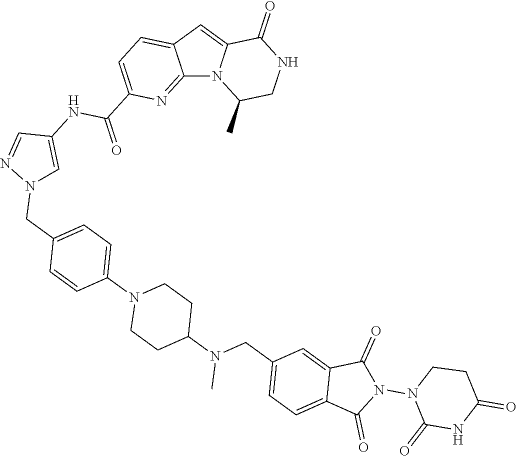 [SMILES: C[C@@H]1CNC(=O)c2cc3ccc(C(=O)Nc4cnn(Cc5ccc(N6CCC(N(C)Cc7ccc8c(c7)C(=O)N(N7CCC(=O)NC7=O)C8=O)CC6)cc5)c4)nc3n21] | (R)-N-(1-(4-(4-(((2-(2,4- dioxotetrahydropyrimidin-1(2H)-yl)-1,3- dioxoisoindolin-5- yl)methyl)(methyl)amino)piperidin-1- yl)benzyl)-1H-pyrazol-4-yl)-9-methyl-6-oxo- 6,7,8,9-tetrahydropyrido[3′,2′:4,5]pyrrolo[1,2- a]pyrazine-2-carboxamide |
| GT- 0815 2 | 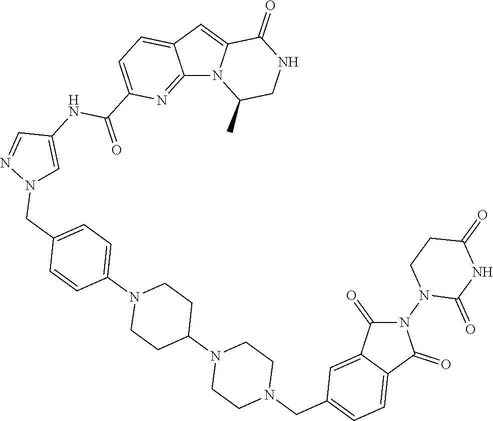 [SMILES: C[C@@H]1CNC(=O)c2cc3ccc(C(=O)Nc4cnn(Cc5ccc(N6CCC(N7CCN(Cc8ccc9c(c8)C(=O)N(N8CCC(=O)NC8=O)C9=O)CC7)CC6)cc5)c4)nc3n21] | (R)-N-(1-(4-(4-(4-((2-(2,4- dioxotetrahydropyrimidin-1(2H)-yl)-1,3- dioxoisoindolin-5-yl)methyl)piperazin-1- yl)piperidin-1-yl)benzyl)-1H-pyrazol-4-yl)-9- methyl-6-oxo-6,7,8,9- tetrahydropyrido [3′,2′:4,5]pyrrolo[1,2- a]pyrazine-2-carboxamide |
| GT- 0507 1 | 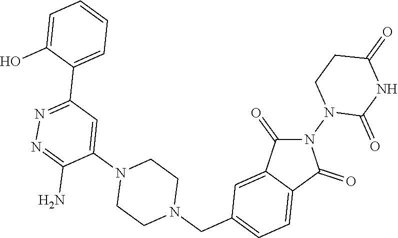 [SMILES: Nc1nnc(-c2ccccc2O)cc1N1CCN(Cc2ccc3c(c2)C(=O)N(N2CCC(=O)NC2=O)C3=O)CC1] | 5-((4-(3-amino-6-(2- hydroxyphenyl)pyridazin-4-yl)piperazin-1- yl)methyl)-2-(2,4-dioxotetrahydropyrimidin- 1(2H)-yl)isoindoline-1,3-dione |
| GT- 0495 4 | 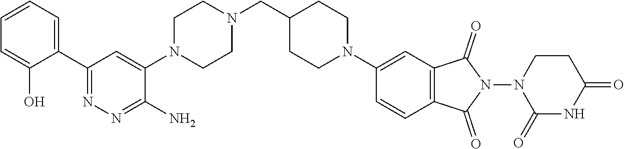 [SMILES: Nc1nnc(-c2ccccc2O)cc1N1CCN(CC2CCN(c3ccc4c(c3)C(=O)N(N3CCC(=O)NC3=O)C4=O)CC2)CC1] | 5-(4-((4-(3-amino-6-(2- hydroxyphenyl)pyridazin-4-yl)piperazin-1- yl)methyl)piperidin-1-yl)-2-(2,4- dioxotetrahydropyrimidin-1(2H)- yl)isoindoline-1,3-dione |
| GT- 0507 0 | 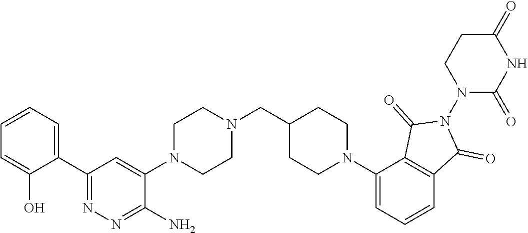 [SMILES: Nc1nnc(-c2ccccc2O)cc1N1CCN(CC2CCN(c3cccc4c3C(=O)N(N3CCC(=O)NC3=O)C4=O)CC2)CC1] | 4-(4-((4-(3-amino-6-(2- hydroxyphenyl)pyridazin-4-yl)piperazin-1- yl)methyl)piperidin-1-yl)-2-(2,4- dioxotetrahydropyrimidin-1(2H)- yl)isoindoline-1,3-dione |
| GT- 0509 5 | 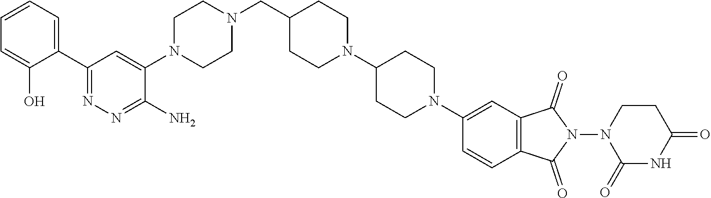 [SMILES: Nc1nnc(-c2ccccc2O)cc1N1CCN(CC2CCN(C3CCN(c4ccc5c(c4)C(=O)N(N4CCC(=O)NC4=O)C5=O)CC3)CC2)CC1] | 5-(4-((4-(3-amino-6-(2- hydroxyphenyl)pyridazin-4-yl)piperazin-1- yl)methyl)-[1,4′-bipiperidin]-1′-yl)-2-(2,4- dioxotetrahydropyrimidin-1(2H)- yl)isoindoline-1,3-dione |
| GT- 0507 2 | 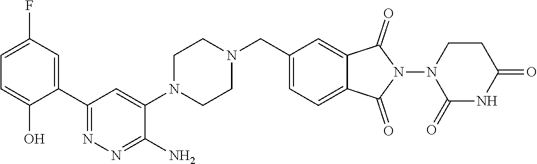 [SMILES: Nc1nnc(-c2cc(F)ccc2O)cc1N1CCN(Cc2ccc3c(c2)C(=O)N(N2CCC(=O)NC2=O)C3=O)CC1] | 5-((4-(3-amino-6-(5-fluoro-2- hydroxyphenyl)pyridazin-4-yl)piperazin-1- yl)methyl)-2-(2,4-dioxotetrahydropyrimidin- 1(2H)-yl)isoindoline-1,3-dione |
| GT- 0506 8 | 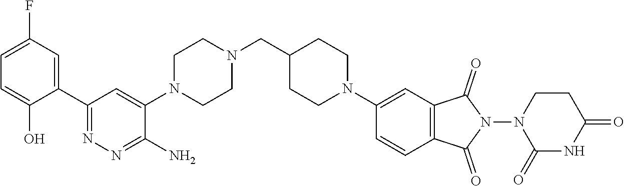 [SMILES: Nc1nnc(-c2cc(F)ccc2O)cc1N1CCN(CC2CCN(c3ccc4c(c3)C(=O)N(N3CCC(=O)NC3=O)C4=O)CC2)CC1] | 5-(4-((4-(3-amino-6-(5-fluoro-2- hydroxyphenyl)pyridazin-4-yl)piperazin-1- yl)methyl)piperidin-1-yl)-2-(2,4- dioxotetrahydropyrimidin-1(2H)- yl)isoindoline-1,3-dione |
| GT- 0506 9 | 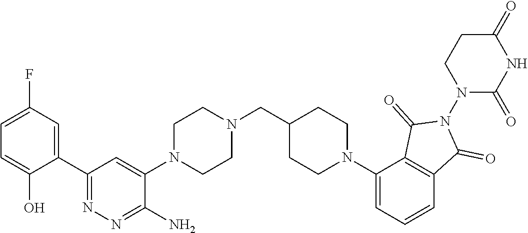 [SMILES: Nc1nnc(-c2cc(F)ccc2O)cc1N1CCN(CC2CCN(c3cccc4c3C(=O)N(N3CCC(=O)NC3=O)C4=O)CC2)CC1] | 4-(4-((4-(3-amino-6-(5-fluoro-2- hydroxyphenyl)pyridazin-4-yl)piperazin-1- yl)methyl)piperidin-1-yl)-2-(2,4- dioxotetrahydropyrimidin-1(2H)- yl)isoindoline-1,3-dione |
| GT- 0509 4 | 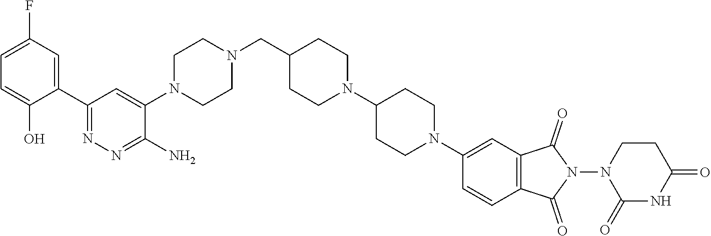 [SMILES: Nc1nnc(-c2cc(F)ccc2O)cc1N1CCN(CC2CCN(C3CCN(c4ccc5c(c4)C(=O)N(N4CCC(=O)NC4=O)C5=O)CC3)CC2)CC1] | 5-(4-((4-(3-amino-6-(5-fluoro-2- hydroxyphenyl)pyridazin-4-yl)piperazin-1- yl)methyl)-[1,4′-bipiperidin]-1′-yl)-2-(2,4- dioxotetrahydropyrimidin-1(2H)- yl)isoindoline-1,3-dione |
| GT- 0584 3 | 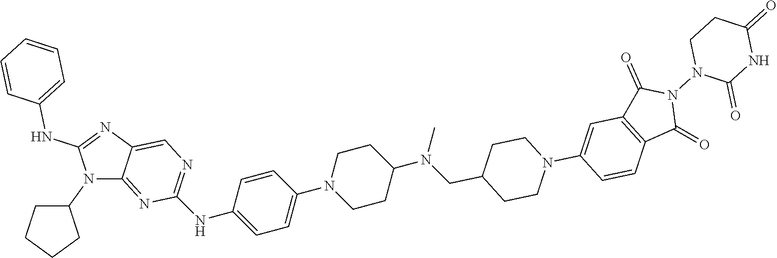 [SMILES: CN(CC1CCN(c2ccc3c(c2)C(=O)N(N2CCC(=O)NC2=O)C3=O)CC1)C1CCN(c2ccc(Nc3ncc4nc(Nc5ccccc5)n(C5CCCC5)c4n3)cc2)CC1] | 5-(4-(((1-(4-((9-cyclopentyl-8-(phenylamino)- 9H-purin-2-yl)amino)phenyl)piperidin-4- yl)(methyl)amino)methyl)piperidin-1-yl)-2- (2,4-dioxotetrahydropyrimidin-1(2H)- yl)isoindoline-1,3-dione |
| GT- 0584 4 | 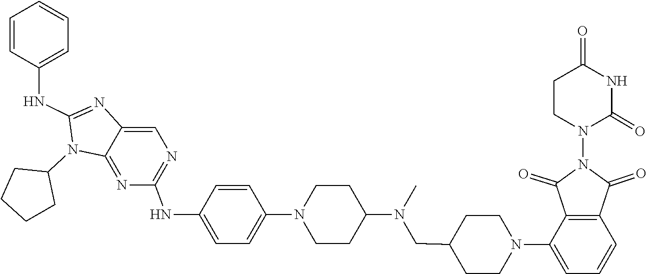 [SMILES: CN(CC1CCN(c2cccc3c2C(=O)N(N2CCC(=O)NC2=O)C3=O)CC1)C1CCN(c2ccc(Nc3ncc4nc(Nc5ccccc5)n(C5CCCC5)c4n3)cc2)CC1] | 4-(4-(((1-(4-((9-cyclopentyl-8-(phenylamino)- 9H-purin-2-yl)amino)phenyl)piperidin-4- yl)(methyl)amino)methyl)piperidin-1-yl)-2- (2,4-dioxotetrahydropyrimidin-1(2H)- yl)isoindoline-1,3-dione |
| GT- 0595 5 | 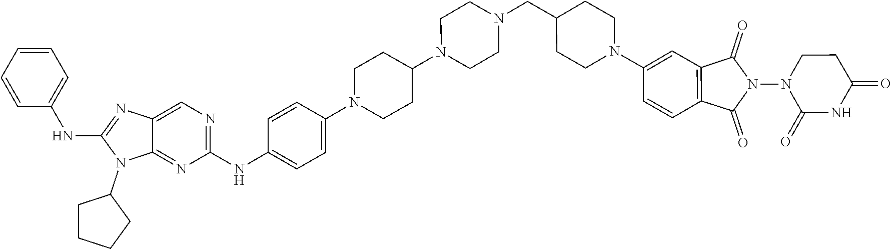 [SMILES: O=C1CCN(N2C(=O)c3ccc(N4CCC(CN5CCN(C6CCN(c7ccc(Nc8ncc9nc(Nc%10ccccc%10)n(C%10CCCC%10)c9n8)cc7)CC6)CC5)CC4)cc3C2=O)C(=O)N1] | 5-(4-((4-(1-(4-((9-cyclopentyl-8- (phenylamino)-9H-purin-2- yl)amino)phenyl)piperidin-4-yl)piperazin-1- yl)methyl)piperidin-1-yl)-2-(2,4- dioxotetrahydropyrimidin-1(2H)- yl)isoindoline-1,3-dione |
| GT- 0595 6 | 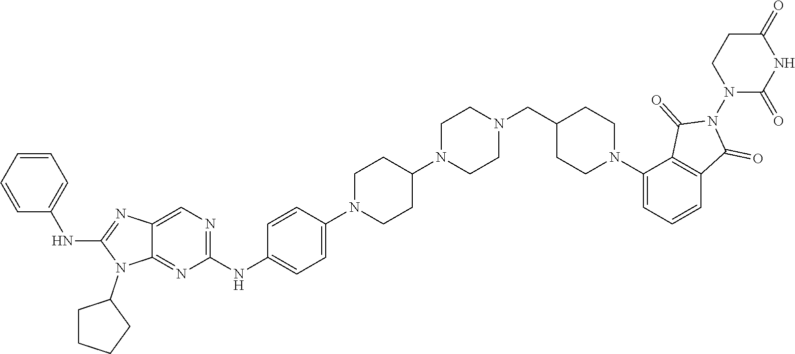 [SMILES: O=C1CCN(N2C(=O)c3cccc(N4CCC(CN5CCN(C6CCN(c7ccc(Nc8ncc9nc(Nc%10ccccc%10)n(C%10CCCC%10)c9n8)cc7)CC6)CC5)CC4)c3C2=O)C(=O)N1] | 4-(4-((4-(1-(4-((9-cyclopentyl-8- (phenylamino)-9H-purin-2- yl)amino)phenyl)piperidin-4-yl)piperazin-1- yl)methyl)piperidin-1-yl)-2-(2,4- dioxotetrahydropyrimidin-1(2H)- yl)isoindoline-1,3-dione |
| GT- 0594 9 | 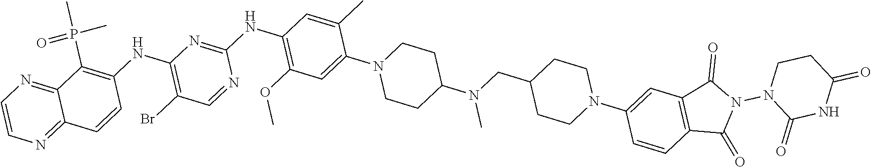 [SMILES: COc1cc(N2CCC(N(C)CC3CCN(c4ccc5c(c4)C(=O)N(N4CCC(=O)NC4=O)C5=O)CC3)CC2)c(C)cc1Nc1ncc(Br)c(Nc2ccc3nccnc3c2P(C)(C)=O)n1] | 5-(4-(((1-(4-((5-bromo-4-((5- (dimethylphosphoryl)quinoxalin-6- yl)amino)pyrimidin-2-yl)amino)-5-methoxy- 2-methylphenyl)piperidin-4- yl)(methyl)amino)methyl)piperidin-1-yl)-2- (2,4-dioxotetrahydropyrimidin-1(2H)- yl)isoindoline-1,3-dione |
| GT- 0595 0 | 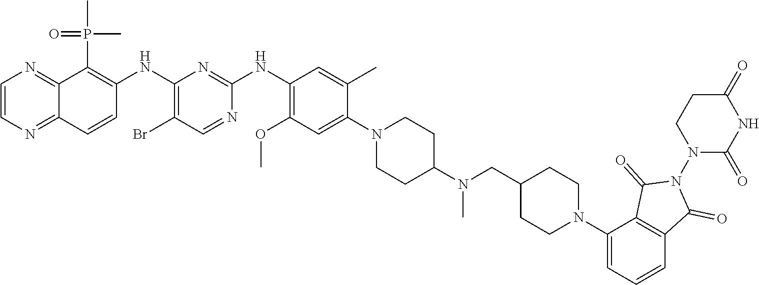 [SMILES: COc1cc(N2CCC(N(C)CC3CCN(c4cccc5c4C(=O)N(N4CCC(=O)NC4=O)C5=O)CC3)CC2)c(C)cc1Nc1ncc(Br)c(Nc2ccc3nccnc3c2P(C)(C)=O)n1] | 4-(4-(((1-(4-((5-bromo-4-((5- (dimethylphosphoryl)quinoxalin-6- yl)amino)pyrimidin-2-yl)amino)-5-methoxy- 2-methylphenyl)piperidin-4- yl)(methyl)amino)methyl)piperidin-1-yl)-2- (2,4-dioxotetrahydropyrimidin-1(2H)- yl)isoindoline-1,3-dione |
| GT- 0599 7 | 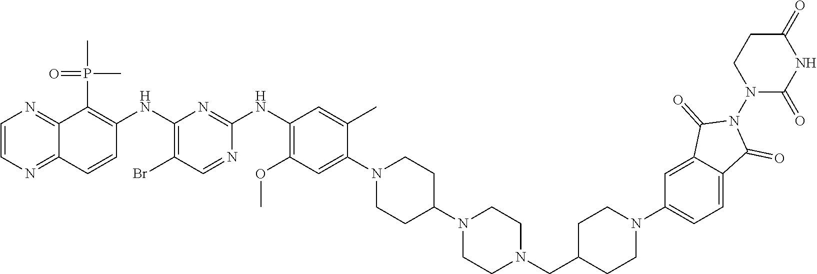 [SMILES: COc1cc(N2CCC(N3CCN(CC4CCN(c5ccc6c(c5)C(=O)N(N5CCC(=O)NC5=O)C6=O)CC4)CC3)CC2)c(C)cc1Nc1ncc(Br)c(Nc2ccc3nccnc3c2P(C)(C)=O)n1] | 5-(4-((4-(1-(4-((5-bromo-4-((5- (dimethylphosphoryl)quinoxalin-6- yl)amino)pyrimidin-2-yl)amino)-5-methoxy- 2-methylphenyl)piperidin-4-yl)piperazin-1- yl)methyl)piperidin-1-yl)-2-(2,4- dioxotetrahydropyrimidin-1(2H)- yl)isoindoline-1,3-dione |
| GT- 0599 8 | 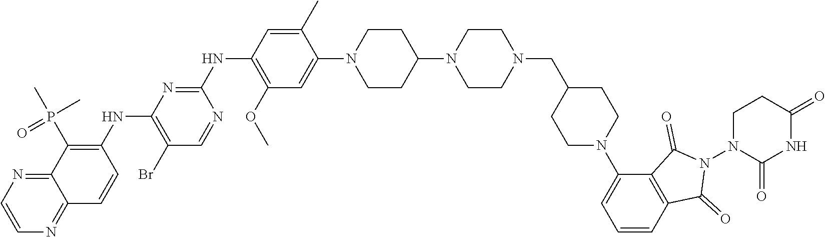 [SMILES: COc1cc(N2CCC(N3CCN(CC4CCN(c5cccc6c5C(=O)N(N5CCC(=O)NC5=O)C6=O)CC4)CC3)CC2)c(C)cc1Nc1ncc(Br)c(Nc2ccc3nccnc3c2P(C)(C)=O)n1] | 4-(4-((4-(1-(4-((5-bromo-4-((5- (dimethylphosphoryl)quinoxalin-6- yl)amino)pyrimidin-2-yl)amino)-5-methoxy- 2-methylphenyl)piperidin-4-yl)piperazin-1- yl)methyl)piperidin-1-yl)-2-(2,4- dioxotetrahydropyrimidin-1(2H)- yl)isoindoline-1,3-dione |
| GT- 0607 3 | 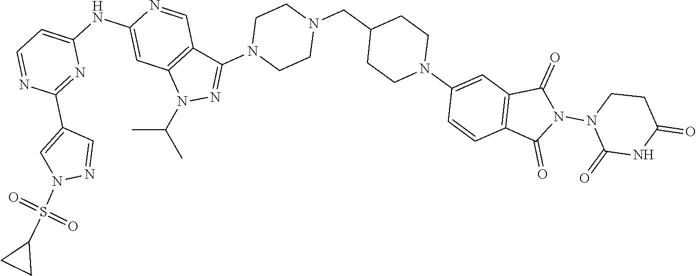 [SMILES: CC(C)n1nc(N2CCN(CC3CCN(c4ccc5c(c4)C(=O)N(N4CCC(=O)NC4=O)C5=O)CC3)CC2)c2cnc(Nc3ccnc(-c4cnn(S(=O)(=O)C5CC5)c4)n3)cc21] | 5-(4-((4-(6-((2-(1-(cyclopropylsulfonyl)-1H- pyrazol-4-yl)pyrimidin-4-yl)amino)-1- isopropyl-1H-pyrazolo[4,3-c]pyridin-3- yl)piperazin-1-yl)methyl)piperidin-1-yl)-2- (2,4-dioxotetrahydropyrimidin-1(2H)- yl)isoindoline-1,3-dione |
| GT- 0607 5 | 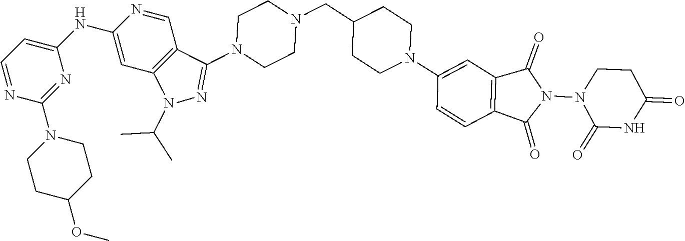 [SMILES: COC1CCN(c2nccc(Nc3cc4c(cn3)c(N3CCN(CC5CCN(c6ccc7c(c6)C(=O)N(N6CCC(=O)NC6=O)C7=O)CC5)CC3)nn4C(C)C)n2)CC1] | 2-(2,4-dioxotetrahydropyrimidin-1(2H)-yl)-5- (4-((4-(1-isopropyl-6-((2-(4- methoxypiperidin-1-yl)pyrimidin-4- yl)amino)-1H-pyrazolo[4,3-c]pyridin-3- yl)piperazin-1-yl)methyl)piperidin-1- yl)isoindoline-1,3-dione |
| GT- 0603 5 | 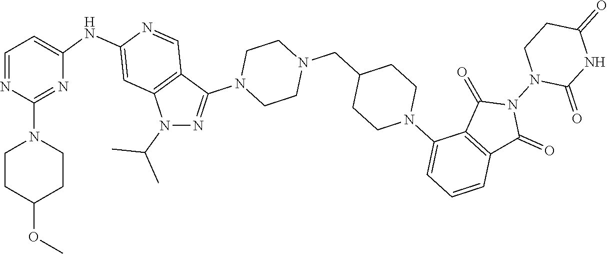 [SMILES: COC1CCN(c2nccc(Nc3cc4c(cn3)c(N3CCN(CC5CCN(c6cccc7c6C(=O)N(N6CCC(=O)NC6=O)C7=O)CC5)CC3)nn4C(C)C)n2)CC1] | 2-(2,4-dioxotetrahydropyrimidin-1(2H)-yl)-4- (4-((4-(1-isopropyl-6-((2-(4- methoxypiperidin-1-yl)pyrimidin-4- yl)amino)-1H-pyrazolo[4,3-c]pyridin-3- yl)piperazin-1-yl)methyl)piperidin-1- yl)isoindoline-1,3-dione |
| GT- 0611 2 | 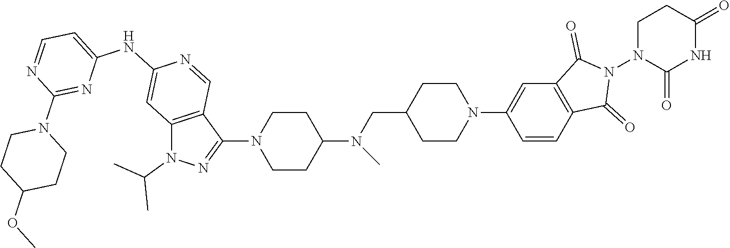 [SMILES: COC1CCN(c2nccc(Nc3cc4c(cn3)c(N3CCC(N(C)CC5CCN(c6ccc7c(c6)C(=O)N(N6CCC(=O)NC6=O)C7=O)CC5)CC3)nn4C(C)C)n2)CC1] | 2-(2,4-dioxotetrahydropyrimidin-1(2H)-yl)-5- (4-(((1-(1-isopropyl-6-((2-(4- methoxypiperidin-1-yl)pyrimidin-4- yl)amino)-1H-pyrazolo[4,3-c]pyridin-3- yl)piperidin-4- yl)(methyl)amino)methyl)piperidin-1- yl)isoindoline-1,3-dione |
| GT- 0611 9 | 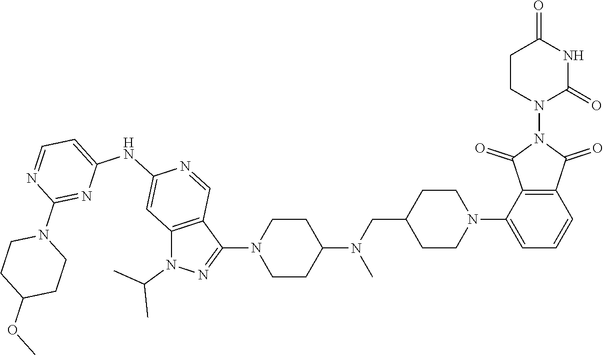 [SMILES: COC1CCN(c2nccc(Nc3cc4c(cn3)c(N3CCC(N(C)CC5CCN(c6cccc7c6C(=O)N(N6CCC(=O)NC6=O)C7=O)CC5)CC3)nn4C(C)C)n2)CC1] | 2-(2,4-dioxotetrahydropyrimidin-1(2H)-yl)-4- (4-(((1-(1-isopropyl-6-((2-(4- methoxypiperidin-1-yl)pyrimidin-4- yl)amino)-1H-pyrazolo[4,3-c]pyridin-3- yl)piperidin-4- yl)(methyl)amino)methyl)piperidin-1- yl)isoindoline-1,3-dione |
| GT- 0616 1 | 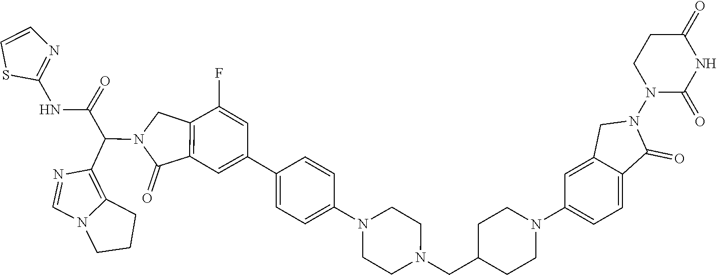 [SMILES: O=C1CCN(N2Cc3cc(N4CCC(CN5CCN(c6ccc(-c7cc(F)c8c(c7)C(=O)N(C(C(=O)Nc7nccs7)c7ncn9c7CCC9)C8)cc6)CC5)CC4)ccc3C2=O)C(=O)N1] | 2-(6,7-dihydro-5H-pyrrolo[1,2-c]imidazol-1- yl)-2-(6-(4-(4-((1-(2-(2,4- dioxotetrahydropyrimidin-1(2H)-yl)-1,3- dioxoisoindolin-5-yl)piperidin-4- yl)methyl)piperazin-1-yl)phenyl)-4-fluoro-1- oxoisoindolin-2-yl)-N-(thiazol-2- yl)acetamide |
| GT- 0616 2 | 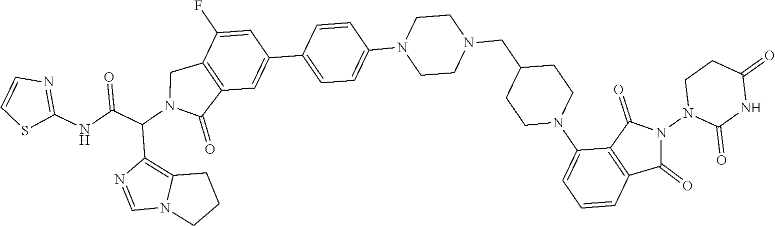 [SMILES: O=C1CCN(N2C(=O)c3cccc(N4CCC(CN5CCN(c6ccc(-c7cc(F)c8c(c7)C(=O)N(C(C(=O)Nc7nccs7)c7ncn9c7CCC9)C8)cc6)CC5)CC4)c3C2=O)C(=O)N1] | 2-(6,7-dihydro-5H-pyrrolo[1,2-c]imidazol-1- yl)-2-(6-(4-(4-((1-(2-(2,4- dioxotetrahydropyrimidin-1(2H)-yl)-1,3- dioxoisoindolin-4-yl)piperidin-4- yl)methyl)piperazin-1-yl)phenyl)-4-fluoro-1- oxoisoindolin-2-yl)-N-(thiazol-2- yl)acetamide |
| GT- 0617 4 | 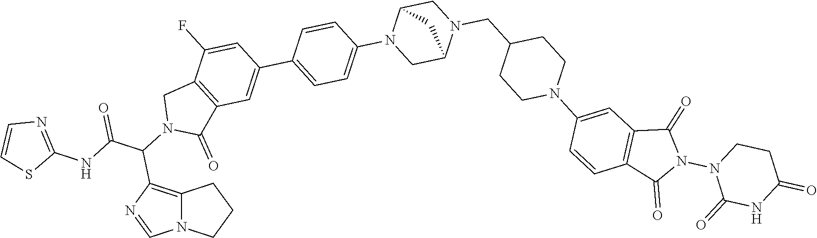 [SMILES: O=C1CCN(N2C(=O)c3ccc(N4CCC(CN5C[C@H]6C[C@@H]5CN6c5ccc(-c6cc(F)c7c(c6)C(=O)N(C(C(=O)Nc6nccs6)c6ncn8c6CCC8)C7)cc5)CC4)cc3C2=O)C(=O)N1] | 2-(6,7-dihydro-5H-pyrrolo[1,2-c]imidazol-1- yl)-2-(6-(4-((1R,4R)-5-((1-(2-(2,4- dioxotetrahydropyrimidin-1(2H)-yl)-1,3- dioxoisoindolin-5-yl)piperidin-4-yl)methyl)- 2,5-diazabicyclo[2.2.1]heptan-2-yl)phenyl)-4- fluoro-1-oxoisoindolin-2-yl)-N-(thiazol-2- yl)acetamide |
| GT- 0617 5 | 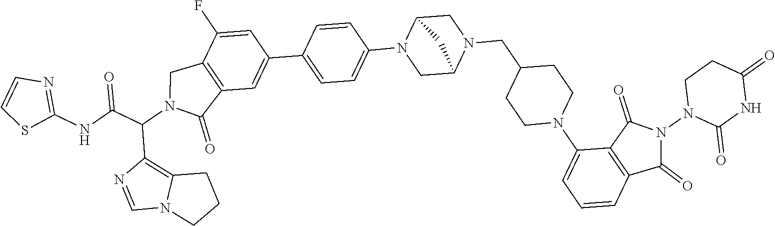 [SMILES: O=C1CCN(N2C(=O)c3cccc(N4CCC(CN5C[C@H]6C[C@@H]5CN6c5ccc(-c6cc(F)c7c(c6)C(=O)N(C(C(=O)Nc6nccs6)c6ncn8c6CCC8)C7)cc5)CC4)c3C2=O)C(=O)N1] | 2-(6,7-dihydro-5H-pyrrolo[1,2-c]imidazol-1- yl)-2-(6-(4-((1R,4R)-5-((1-(2-(2,4- dioxotetrahydropyrimidin-1(2H)-yl)-1,3- dioxoisoindolin-4-yl)piperidin-4-yl)methyl)- 2,5-diazabicyclo[2.2.1]heptan-2-yl)phenyl)-4- fluoro-1-oxoisoindolin-2-yl)-N-(thiazol-2- yl)acetamide |
| GT- 0471 9 | 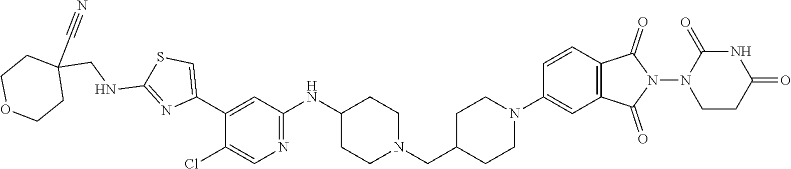 [SMILES: N#CC1(CNc2nc(-c3cc(NC4CCN(CC5CCN(c6ccc7c(c6)C(=O)N(N6CCC(=O)NC6=O)C7=O)CC5)CC4)ncc3Cl)cs2)CCOCC1] | 4-(((4-(5-chloro-2-((1-((1-(2-(2,4- dioxotetrahydropyrimidin-1(2H)-yl)-1,3- dioxoisoindolin-5-yl)piperidin-4- yl)methyl)piperidin-4-yl)amino)pyridin-4- yl)thiazol-2-yl)amino)methyl)tetrahydro-2H- pyran-4-carbonitrile |
| GT- 0834 5 | 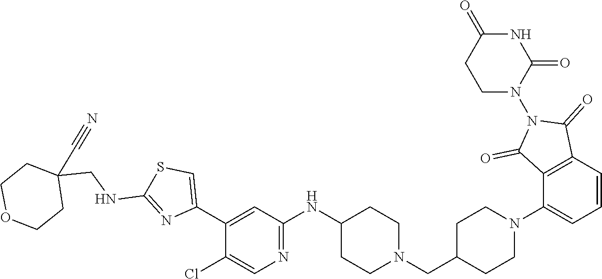 [SMILES: N#CC1(CNc2nc(-c3cc(NC4CCN(CC5CCN(c6cccc7c6C(=O)N(N6CCC(=O)NC6=O)C7=O)CC5)CC4)ncc3Cl)cs2)CCOCC1] | 4-(((4-(5-chloro-2-((1-((1-(2-(2,4- dioxotetrahydropyrimidin-1(2H)-yl)-1,3- dioxoisoindolin-4-yl)piperidin-4- yl)methyl)piperidin-4-yl)amino)pyridin-4- yl)thiazol-2-yl)amino)methyl)tetrahydro-2H- pyran-4-carbonitrile |
| GT- 0834 6 | 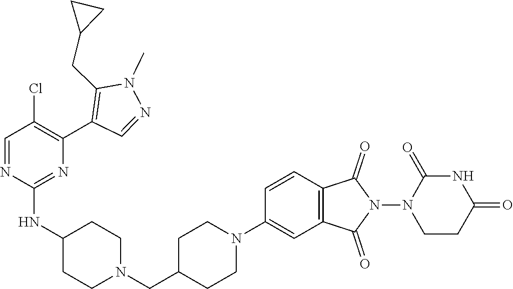 [SMILES: Cn1ncc(-c2nc(NC3CCN(CC4CCN(c5ccc6c(c5)C(=O)N(N5CCC(=O)NC5=O)C6=O)CC4)CC3)ncc2Cl)c1CC1CC1] | 5-(4-((4-((5-chloro-4-(5-(cyclopropylmethyl)- 1-methyl-1H-pyrazol-4-yl)pyrimidin-2- yl)amino)piperidin-1-yl)methyl)piperidin-1- yl)-2-(2,4-dioxotetrahydropyrimidin-1(2H)- yl)isoindoline-1,3-dione |
| GT- 0834 7 | 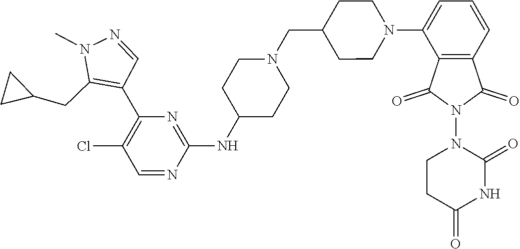 [SMILES: Cn1ncc(-c2nc(NC3CCN(CC4CCN(c5cccc6c5C(=O)N(N5CCC(=O)NC5=O)C6=O)CC4)CC3)ncc2Cl)c1CC1CC1] | 4-(4-((4-((5-chloro-4-(5-(cyclopropylmethyl)- 1-methyl-1H-pyrazol-4-yl)pyrimidin-2- yl)amino)piperidin-1-yl)methyl)piperidin-1- yl)-2-(2,4-dioxotetrahydropyrimidin-1(2H)- yl)isoindoline-1,3-dione |
| GT- 0834 8 | 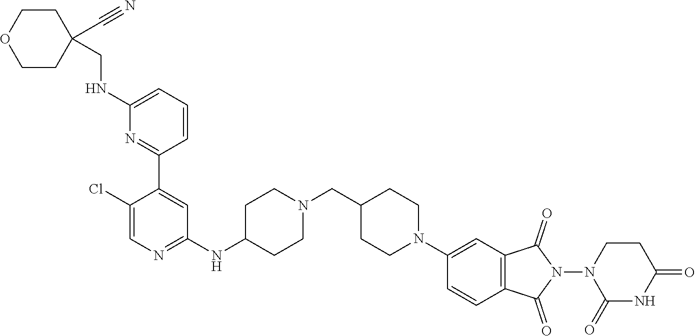 [SMILES: N#CC1(CNc2cccc(-c3cc(NC4CCN(CC5CCN(c6ccc7c(c6)C(=O)N(N6CCC(=O)NC6=O)C7=O)CC5)CC4)ncc3Cl)n2)CCOCC1] | 4-(((5′-chloro-2′-((1-((1-(2-(2,4- dioxotetrahydropyrimidin-1(2H)-yl)-1,3- dioxoisoindolin-5-yl)piperidin-4- yl)methyl)piperidin-4-yl)amino)-[2,4′- bipyridin]-6-yl)amino)methyl)tetrahydro-2H- pyran-4-carbonitrile |
| GT- 0834 9 | 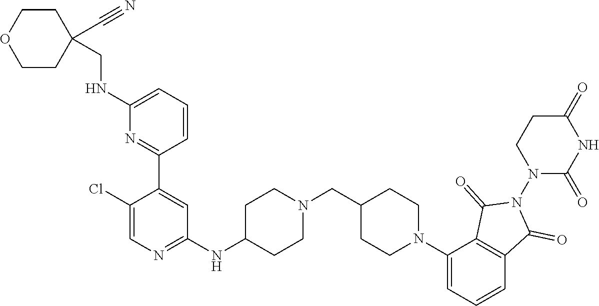 [SMILES: N#CC1(CNc2cccc(-c3cc(NC4CCN(CC5CCN(c6cccc7c6C(=O)N(N6CCC(=O)NC6=O)C7=O)CC5)CC4)ncc3Cl)n2)CCOCC1] | 4-(((5′-chloro-2′-((1-((1-(2-(2,4- dioxotetrahydropyrimidin-1(2H)-yl)-1,3- dioxoisoindolin-4-yl)piperidin-4- yl)methyl)piperidin-4-yl)amino)-[2,4′- bipyridin]-6-yl)amino)methyl)tetrahydro-2H- pyran-4-carbonitrile |
| GT- 0822 8 | 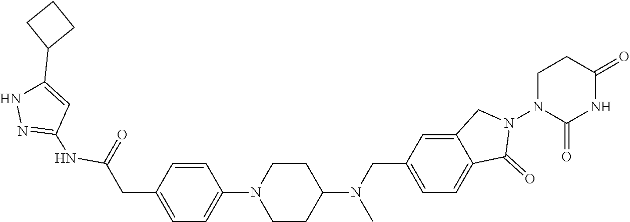 [SMILES: CN(Cc1ccc2c(c1)CN(N1CCC(=O)NC1=O)C2=O)C1CCN(c2ccc(CC(=O)Nc3cc(C4CCC4)[nH]n3)cc2)CC1] | N-(5-cyclobutyl-1H-pyrazol-3-yl)-2-(4-(4- (((2-(2,4-dioxotetrahydropyrimidin-1(2H)- yl)-1-oxoisoindolin-5- yl)methyl)(methyl)amino)piperidin-1- yl)phenyl)acetamide |
| GT- 0824 4 | 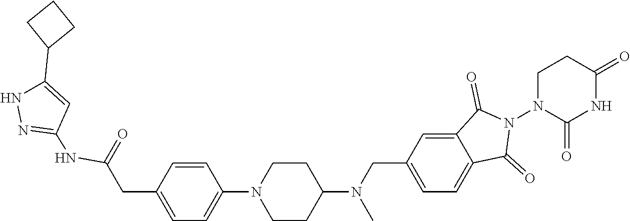 [SMILES: CN(Cc1ccc2c(c1)C(=O)N(N1CCC(=O)NC1=O)C2=O)C1CCN(c2ccc(CC(=O)Nc3cc(C4CCC4)[nH]n3)cc2)CC1] | N-(5-cyclobutyl-1H-pyrazol-3-yl)-2-(4-(4- (((2-(2,4-dioxotetrahydropyrimidin-1(2H)- yl)-1,3-dioxoisoindolin-5- yl)methyl)(methyl)amino)piperidin-1- yl)phenyl)acetamide |
| GT- 0822 9 | 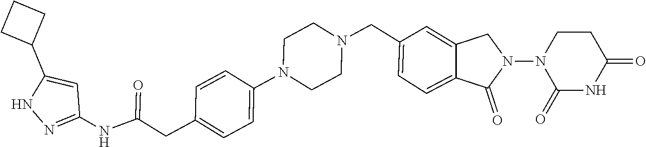 [SMILES: O=C1CCN(N2Cc3cc(CN4CCN(c5ccc(CC(=O)Nc6cc(C7CCC7)[nH]n6)cc5)CC4)ccc3C2=O)C(=O)N1] | N-(5-cyclobutyl-1H-pyrazol-3-yl)-2-(4-(4- ((2-(2,4-dioxotetrahydropyrimidin-1(2H)-yl)- 1-oxoisoindolin-5-yl)methyl)piperazin-1- yl)phenyl)acetamide |
| GT- 0823 0 | 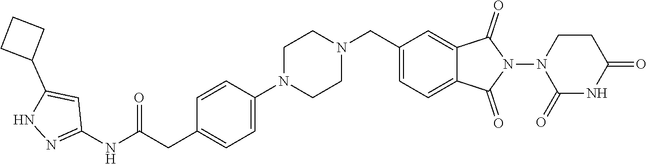 [SMILES: O=C1CCN(N2C(=O)c3ccc(CN4CCN(c5ccc(CC(=O)Nc6cc(C7CCC7)[nH]n6)cc5)CC4)cc3C2=O)C(=O)N1] | N-(5-cyclobutyl-1H-pyrazol-3-yl)-2-(4-(4- ((2-(2,4-dioxotetrahydropyrimidin-1(2H)-yl)- 1,3-dioxoisoindolin-5-yl)methyl)piperazin-1- yl)phenyl)acetamide |
| GT- 0835 0 | 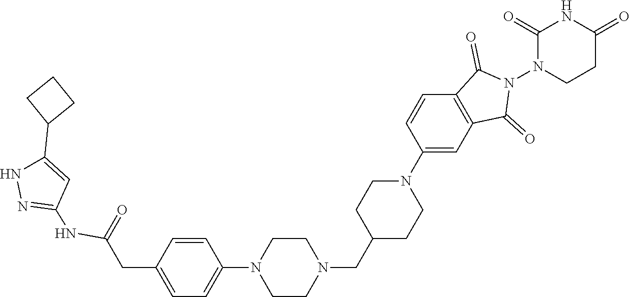 [SMILES: O=C1CCN(N2C(=O)c3ccc(N4CCC(CN5CCN(c6ccc(CC(=O)Nc7cc(C8CCC8)[nH]n7)cc6)CC5)CC4)cc3C2=O)C(=O)N1] | N-(5-cyclobutyl-1H-pyrazol-3-yl)-2-(4-(4- ((1-(2-(2,4-dioxotetrahydropyrimidin-1(2H)- yl)-1,3-dioxoisoindolin-5-yl)piperidin-4- yl)methyl)piperazin-1-yl)phenyl)acetamide |
| GT- 0835 1 | 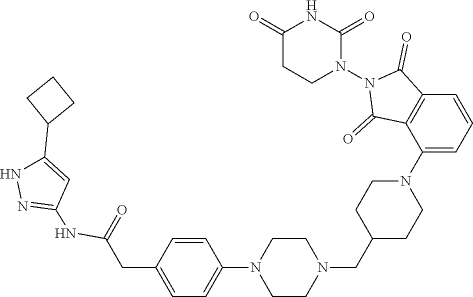 [SMILES: O=C1CCN(N2C(=O)c3cccc(N4CCC(CN5CCN(c6ccc(CC(=O)Nc7cc(C8CCC8)[nH]n7)cc6)CC5)CC4)c3C2=O)C(=O)N1] | N-(5-cyclobutyl-1H-pyrazol-3-yl)-2-(4-(4- ((1-(2-(2,4-dioxotetrahydropyrimidin-1(2H)- yl)-1,3-dioxoisoindolin-4-yl)piperidin-4- yl)methyl)piperazin-1-yl)phenyl)acetamide |
| GT- 0835 2 | 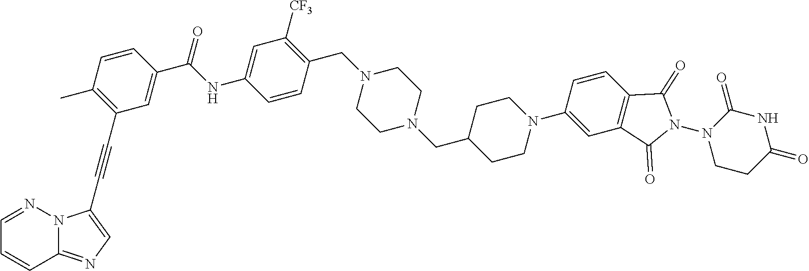 [SMILES: Cc1ccc(C(=O)Nc2ccc(CN3CCN(CC4CCN(c5ccc6c(c5)C(=O)N(N5CCC(=O)NC5=O)C6=O)CC4)CC3)c(C(F)(F)F)c2)cc1C#Cc1cnc2cccnn12] | N-(4-((4-((1-(2-(2,4- dioxotetrahydropyrimidin-1(2H)-yl)-1,3- dioxoisoindolin-5-yl)piperidin-4- yl)methyl)piperazin-1-yl)methyl)-3- (trifluoromethyl)phenyl)-3-(imidazo[1,2- b]pyridazin-3-ylethynyl)-4-methylbenzamide |
| GT- 0835 3 | 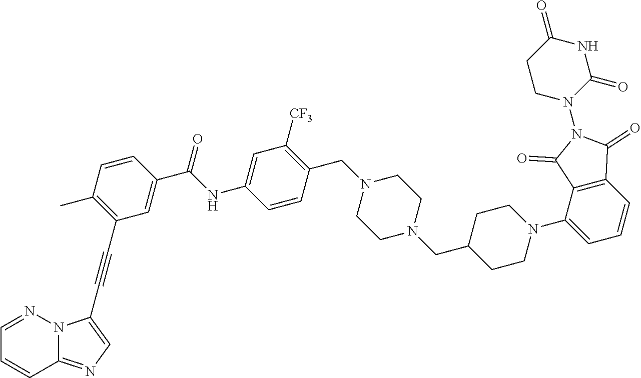 [SMILES: Cc1ccc(C(=O)Nc2ccc(CN3CCN(CC4CCN(c5cccc6c5C(=O)N(N5CCC(=O)NC5=O)C6=O)CC4)CC3)c(C(F)(F)F)c2)cc1C#Cc1cnc2cccnn12] | N-(4-((4-((1-(2-(2,4- dioxotetrahydropyrimidin-1(2H)-yl)-1,3- dioxoisoindolin-4-yl)piperidin-4- yl)methyl)piperazin-1-yl)methyl)-3- (trifluoromethyl)phenyl)-3-(imidazo[1,2- b]pyridazin-3-ylethynyl)-4-methylbenzamide |
| GT- 0835 4 | 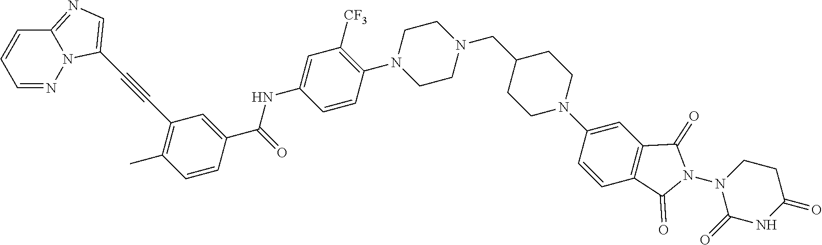 [SMILES: Cc1ccc(C(=O)Nc2ccc(N3CCN(CC4CCN(c5ccc6c(c5)C(=O)N(N5CCC(=O)NC5=O)C6=O)CC4)CC3)c(C(F)(F)F)c2)cc1C#Cc1cnc2cccnn12] | N-(4-(4-((1-(2-(2,4- dioxotetrahydropyrimidin-1(2H)-yl)-1,3- dioxoisoindolin-5-yl)piperidin-4- yl)methyl)piperazin-1-yl)-3- (trifluoromethyl)phenyl)-3-(imidazo[1,2- b]pyridazin-3-ylethynyl)-4-methylbenzamide |
| GT- 0835 5 | 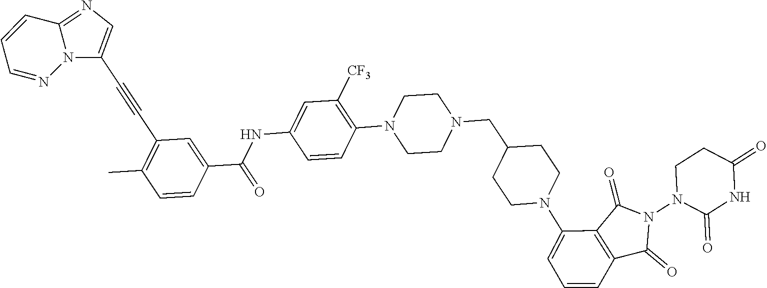 [SMILES: Cc1ccc(C(=O)Nc2ccc(N3CCN(CC4CCN(c5cccc6c5C(=O)N(N5CCC(=O)NC5=O)C6=O)CC4)CC3)c(C(F)(F)F)c2)cc1C#Cc1cnc2cccnn12] | N-(4-(4-((1-(2-(2,4- dioxotetrahydropyrimidin-1(2H)-yl)-1,3- dioxoisoindolin-4-yl)piperidin-4- yl)methyl)piperazin-1-yl)-3- (trifluoromethyl)phenyl)-3-(imidazo[1,2- b]pyridazin-3-ylethynyl)-4-methylbenzamide |
| GT- 0835 6 | 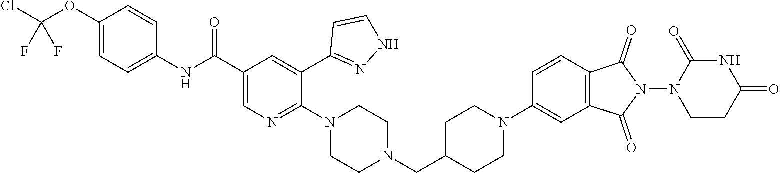 [SMILES: O=C1CCN(N2C(=O)c3ccc(N4CCC(CN5CCN(c6ncc(C(=O)Nc7ccc(OC(F)(F)Cl)cc7)cc6-c6cc[nH]n6)CC5)CC4)cc3C2=O)C(=O)N1] | N-(4-(chlorodifluoromethoxy)phenyl)-6-(4- ((1-(2-(2,4-dioxotetrahydropyrimidin-1(2H)- yl)-1,3-dioxoisoindolin-5-yl)piperidin-4- yl)methyl)piperazin-1-yl)-5-(1H-pyrazol-3- yl)nicotinamide |
| GT- 0835 7 | 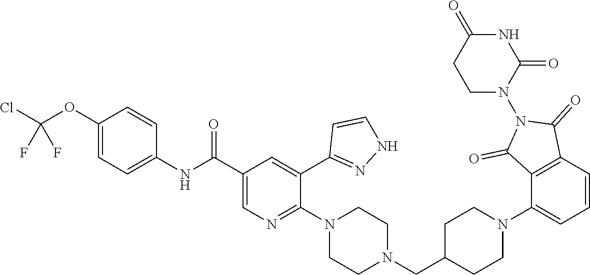 [SMILES: O=C1CCN(N2C(=O)c3cccc(N4CCC(CN5CCN(c6ncc(C(=O)Nc7ccc(OC(F)(F)Cl)cc7)cc6-c6cc[nH]n6)CC5)CC4)c3C2=O)C(=O)N1] | N-(4-(chlorodifluoromethoxy)phenyl)-6-(4- ((1-(2-(2,4-dioxotetrahydropyrimidin-1(2H)- yl)-1,3-dioxoisoindolin-4-yl)piperidin-4- yl)methyl)piperazin-1-yl)-5-(1H-pyrazol-3- yl)nicotinamide |
| GT- 0835 8 | 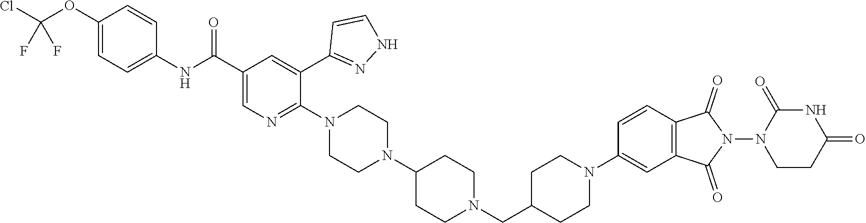 [SMILES: O=C1CCN(N2C(=O)c3ccc(N4CCC(CN5CCC(N6CCN(c7ncc(C(=O)Nc8ccc(OC(F)(F)Cl)cc8)cc7-c7cc[nH]n7)CC6)CC5)CC4)cc3C2=O)C(=O)N1] | N-(4-(chlorodifluoromethoxy)phenyl)-6-(4- (1-((1-(2-(2,4-dioxotetrahydropyrimidin- 1(2H)-yl)-1,3-dioxoisoindolin-5-yl)piperidin- 4-yl)methyl)piperidin-4-yl)piperazin-1-yl)-5- (1H-pyrazol-3-yl)nicotinamide |
| GT- 0835 9 | 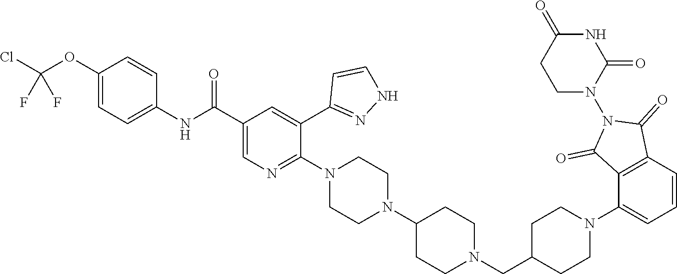 [SMILES: O=C1CCN(N2C(=O)c3cccc(N4CCC(CN5CCC(N6CCN(c7ncc(C(=O)Nc8ccc(OC(F)(F)Cl)cc8)cc7-c7cc[nH]n7)CC6)CC5)CC4)c3C2=O)C(=O)N1] | N-(4-(chlorodifluoromethoxy)phenyl)-6-(4- (1-((1-(2-(2,4-dioxotetrahydropyrimidin- 1(2H)-yl)-1,3-dioxoisoindolin-4-yl)piperidin- 4-yl)methyl)piperidin-4-yl)piperazin-1-yl)-5- (1H-pyrazol-3-yl)nicotinamide |
| GT- 0836 0 | 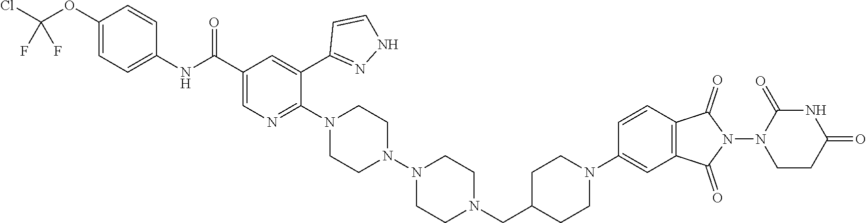 [SMILES: O=C1CCN(N2C(=O)c3ccc(N4CCC(CN5CCN(N6CCN(c7ncc(C(=O)Nc8ccc(OC(F)(F)Cl)cc8)cc7-c7cc[nH]n7)CC6)CC5)CC4)cc3C2=O)C(=O)N1] | N-(4-(chlorodifluoromethoxy)phenyl)-6-(4- (4-((1-(2-(2,4-dioxotetrahydropyrimidin- 1(2H)-yl)-1,3-dioxoisoindolin-5-yl)piperidin- 4-yl)methyl)piperazin-1-yl)piperidin-1-yl)-5- (1H-pyrazol-3-yl)nicotinamide |
| GT- 0836 1 | 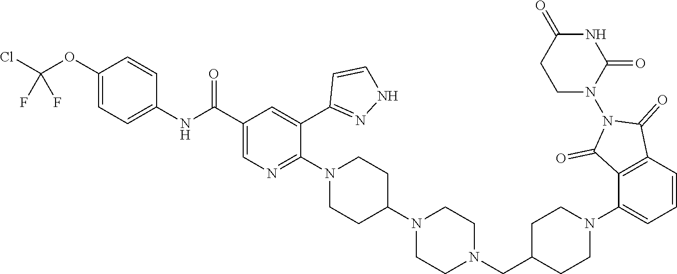 [SMILES: O=C1CCN(N2C(=O)c3cccc(N4CCC(CN5CCN(C6CCN(c7ncc(C(=O)Nc8ccc(OC(F)(F)Cl)cc8)cc7-c7cc[nH]n7)CC6)CC5)CC4)c3C2=O)C(=O)N1] | N-(4-(chlorodifluoromethoxy)phenyl)-6-(4- (4-((1-(2-(2,4-dioxotetrahydropyrimidin- 1(2H)-yl)-1,3-dioxoisoindolin-4-yl)piperidin- 4-yl)methyl)piperazin-1-yl)piperidin-1-yl)-5- (1H-pyrazol-3-yl)nicotinamide |
| GT- 0633 7 | 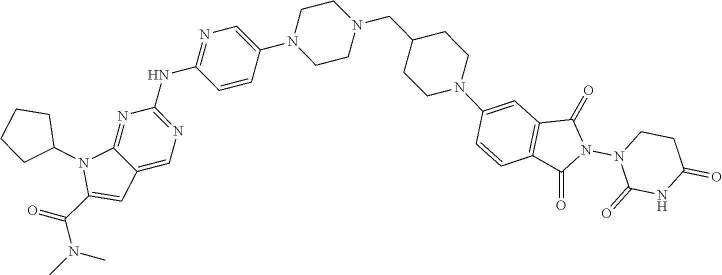 [SMILES: CN(C)C(=O)c1cc2cnc(Nc3ccc(N4CCN(CC5CCN(c6ccc7c(c6)C(=O)N(N6CCC(=O)NC6=O)C7=O)CC5)CC4)cn3)nc2n1C1CCCC1] | 7-cyclopentyl-2-((5-(4-((1-(2-(2,4- dioxotetrahydropyrimidin-1(2H)-yl)-1,3- dioxoisoindolin-5-yl)piperidin-4- yl)methyl)piperazin-1-yl)pyridin-2- yl)amino)-N,N-dimethyl-7H-pyrrolo[2,3- d]pyrimidine-6-carboxamide |
| GT- 0633 8 | 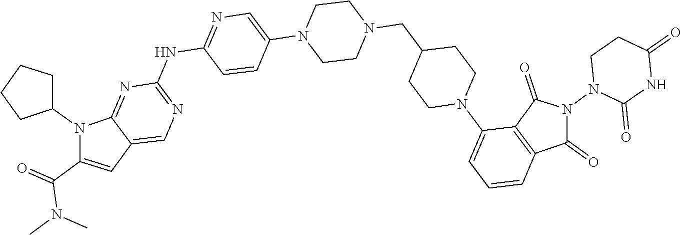 [SMILES: CN(C)C(=O)c1cc2cnc(Nc3ccc(N4CCN(CC5CCN(c6cccc7c6C(=O)N(N6CCC(=O)NC6=O)C7=O)CC5)CC4)cn3)nc2n1C1CCCC1] | 7-cyclopentyl-2-((5-(4-((1-(2-(2,4- dioxotetrahydropyrimidin-1(2H)-yl)-1,3- dioxoisoindolin-4-yl)piperidin-4- yl)methyl)piperazin-1-yl)pyridin-2- yl)amino)-N,N-dimethyl-7H-pyrrolo[2,3- d]pyrimidine-6-carboxamide |
| GT- 0648 9 | 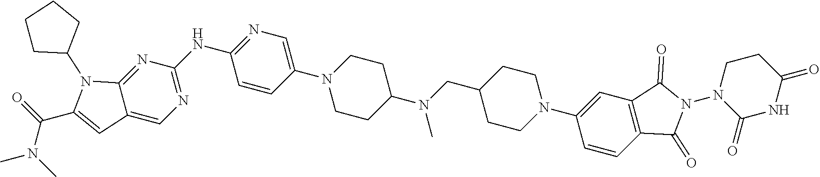 [SMILES: CN(C)C(=O)c1cc2cnc(Nc3ccc(N4CCC(N(C)CC5CCN(c6ccc7c(c6)C(=O)N(N6CCC(=O)NC6=O)C7=O)CC5)CC4)cn3)nc2n1C1CCCC1] | 7-cyclopentyl-2-((5-(4-(((1-(2-(2,4- dioxotetrahydropyrimidin-1(2H)-yl)-1,3- dioxoisoindolin-5-yl)piperidin-4- yl)methyl)(methyl)amino)piperidin-1- yl)pyridin-2-yl)amino)-N,N-dimethyl-7H- pyrrolo[2,3-d]pyrimidine-6-carboxamide |
| GT- 0666 2 | 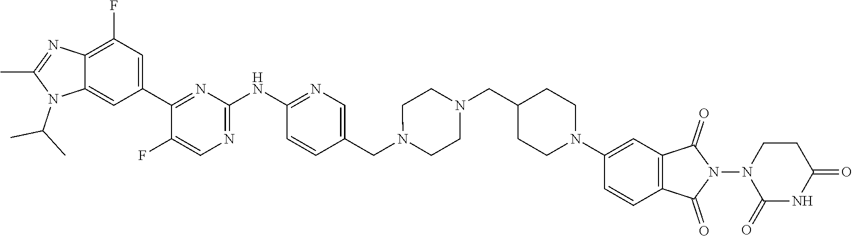 [SMILES: Cc1nc2c(F)cc(-c3nc(Nc4ccc(CN5CCN(CC6CCN(c7ccc8c(c7)C(=O)N(N7CCC(=O)NC7=O)C8=O)CC6)CC5)cn4)ncc3F)cc2n1C(C)C] | 2-(2,4-dioxotetrahydropyrimidin-1(2H)-yl)-5- (4-((4-((6-((5-fluoro-4-(4-fluoro-1-isopropyl- 2-methyl-1H-benzo[d]imidazol-6- yl)pyrimidin-2-yl)amino)pyridin-3- yl)methyl)piperazin-1-yl)methyl)piperidin-1- yl)isoindoline-1,3-dione |
| GT- 0666 3 | 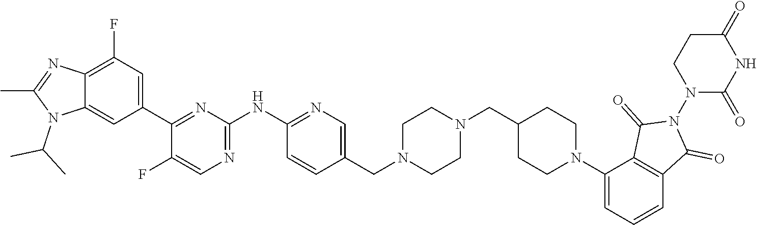 [SMILES: Cc1nc2c(F)cc(-c3nc(Nc4ccc(CN5CCN(CC6CCN(c7cccc8c7C(=O)N(N7CCC(=O)NC7=O)C8=O)CC6)CC5)cn4)ncc3F)cc2n1C(C)C] | 2-(2,4-dioxotetrahydropyrimidin-1(2H)-yl)-4- (4-((4-((6-((5-fluoro-4-(4-fluoro-1-isopropyl- 2-methyl-1H-benzo[d]imidazol-6- yl)pyrimidin-2-yl)amino)pyridin-3- yl)methyl)piperazin-1-yl)methyl)piperidin-1- yl)isoindoline-1,3-dione |
| GT- 0673 3 | 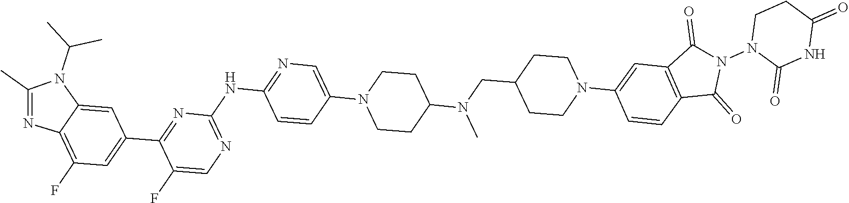 [SMILES: Cc1nc2c(F)cc(-c3nc(Nc4ccc(N5CCC(N(C)CC6CCN(c7ccc8c(c7)C(=O)N(N7CCC(=O)NC7=O)C8=O)CC6)CC5)cn4)ncc3F)cc2n1C(C)C] | 2-(2,4-dioxotetrahydropyrimidin-1(2H)-yl)-5- (4-(((1-(6-((5-fluoro-4-(4-fluoro-1-isopropyl- 2-methyl-1H-benzo[d]imidazol-6- yl)pyrimidin-2-yl)amino)pyridin-3- yl)piperidin-4- yl)(methyl)amino)methyl)piperidin-1- yl)isoindoline-1,3-dione |
| GT- 0673 4 | 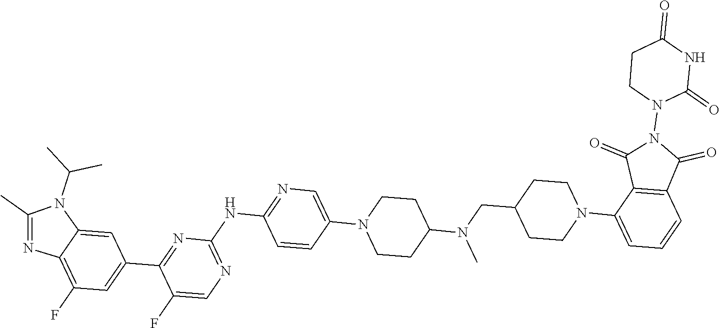 [SMILES: Cc1nc2c(F)cc(-c3nc(Nc4ccc(N5CCC(N(C)CC6CCN(c7cccc8c7C(=O)N(N7CCC(=O)NC7=O)C8=O)CC6)CC5)cn4)ncc3F)cc2n1C(C)C] | 2-(2,4-dioxotetrahydropyrimidin-1(2H)-yl)-4- (4-(((1-(6-((5-fluoro-4-(4-fluoro-1-isopropyl- 2-methyl-1H-benzo[d]imidazol-6- yl)pyrimidin-2-yl)amino)pyridin-3- yl)piperidin-4- yl)(methyl)amino)methyl)piperidin-1- yl)isoindoline-1,3-dione |
| GT- 0582 0 | 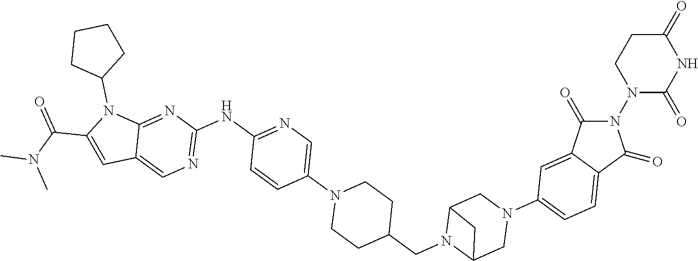 [SMILES: CN(C)C(=O)c1cc2cnc(Nc3ccc(N4CCC(CN5C6CC5CN(c5ccc7c(c5)C(=O)N(N5CCC(=O)NC5=O)C7=O)C6)CC4)cn3)nc2n1C1CCCC1] | 7-cyclopentyl-2-((5-(4-((3-(2-(2,4- dioxotetrahydropyrimidin-1(2H)-yl)-1,3- dioxoisoindolin-5-yl)-3,6- diazabicyclo[3.1.1]heptan-6- yl)methyl)piperidin-1-yl)pyridin-2-yl)amino)- N,N-dimethyl-7H-pyrrolo[2,3-d]pyrimidine- 6-carboxamide |
| GT- 0581 2 | 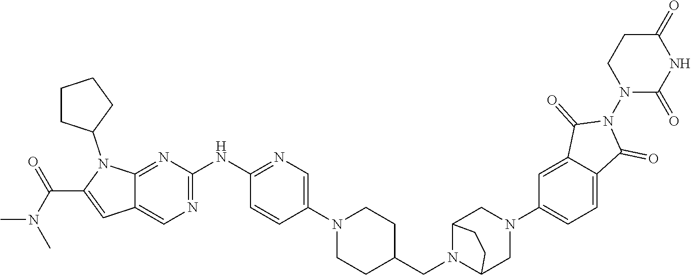 [SMILES: CN(C)C(=O)c1cc2cnc(Nc3ccc(N4CCC(CN5C6CCC5CN(c5ccc7c(c5)C(=O)N(N5CCC(=O)NC5=O)C7=O)C6)CC4)cn3)nc2n1C1CCCC1] | 7-cyclopentyl-2-((5-(4-((3-(2-(2,4- dioxotetrahydropyrimidin-1(2H)-yl)-1,3- dioxoisoindolin-5-yl)-3,8- diazabicyclo[3.2.1]octan-8- yl)methyl)piperidin-1-yl)pyridin-2-yl)amino)- N,N-dimethyl-7H-pyrrolo[2,3-d]pyrimidine- 6-carboxamide |
| GT- 0583 1 | 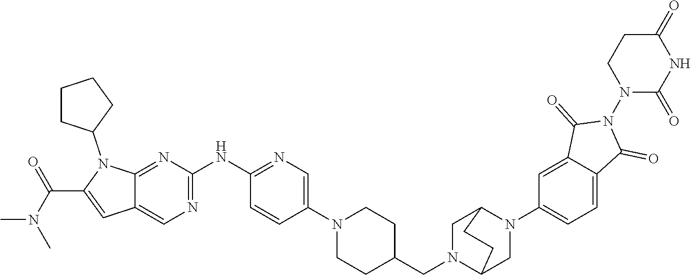 [SMILES: CN(C)C(=O)c1cc2cnc(Nc3ccc(N4CCC(CN5CC6CCC5CN6c5ccc6c(c5)C(=O)N(N5CCC(=O)NC5=O)C6=O)CC4)cn3)nc2n1C1CCCC1] | 7-cyclopentyl-2-((5-(4-((5-(2-(2,4- dioxotetrahydropyrimidin-1(2H)-yl)-1,3- dioxoisoindolin-5-yl)-2,5- diazabicyclo[2.2.2]octan-2- yl)methyl)piperidin-1-yl)pyridin-2-yl)amino)- N,N-dimethyl-7H-pyrrolo[2,3-d]pyrimidine- 6-carboxamide |
| GT- 0682 3 | 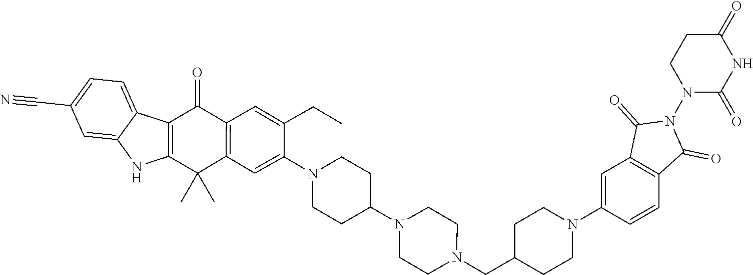 [SMILES: CCc1cc2c(cc1N1CCC(N3CCN(CC4CCN(c5ccc6c(c5)C(=O)N(N5CCC(=O)NC5=O)C6=O)CC4)CC3)CC1)C(C)(C)c1[nH]c3cc(C#N)ccc3c1C2=O] | 8-(4-(4-((1-(2-(2,4-dioxotetrahydropyrimidin- 1(2H)-yl)-1,3-dioxoisoindolin-5-yl)piperidin- 4-yl)methyl)piperazin-1-yl)piperidin-1-yl)-9- ethyl-6,6-dimethyl-11-oxo-6,11-dihydro-5H- benzo[b]carbazole-3-carbonitrile |
| GT- 0729 8 | 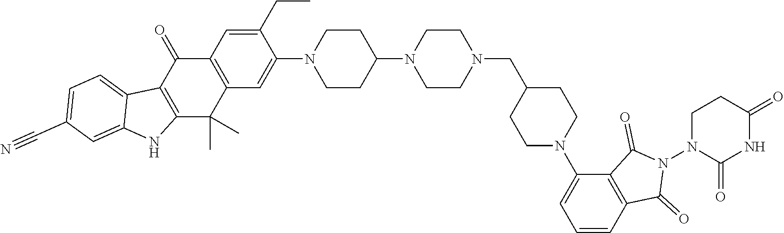 [SMILES: CCc1cc2c(cc1N1CCC(N3CCN(CC4CCN(c5cccc6c5C(=O)N(N5CCC(=O)NC5=O)C6=O)CC4)CC3)CC1)C(C)(C)c1[nH]c3cc(C#N)ccc3c1C2=O] | 8-(4-(4-((1-(2-(2,4-dioxotetrahydropyrimidin- 1(2H)-yl)-1,3-dioxoisoindolin-4-yl)piperidin- 4-yl)methyl)piperazin-1-yl)piperidin-1-yl)-9- ethyl-6,6-dimethyl-11-oxo-6,11-dihydro-5H- benzo[b]carbazole-3-carbonitrile |
| GT- 0732 1 | 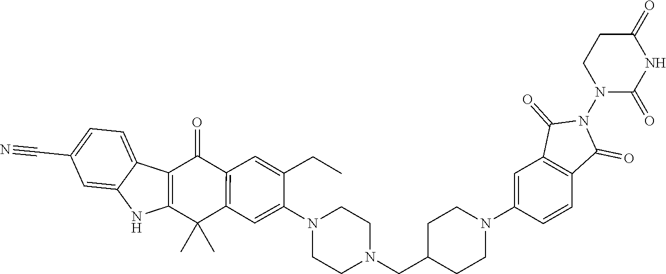 [SMILES: CCc1cc2c(cc1N1CCN(CC3CCN(c4ccc5c(c4)C(=O)N(N4CCC(=O)NC4=O)C5=O)CC3)CC1)C(C)(C)c1[nH]c3cc(C#N)ccc3c1C2=O] | 8-(4-((1-(2-(2,4-dioxotetrahydropyrimidin- 1(2H)-yl)-1,3-dioxoisoindolin-5-yl)piperidin- 4-yl)methyl)piperazin-1-yl)-9-ethyl-6,6- dimethyl-11-oxo-6,11-dihydro-5H- benzo[b]carbazole-3-carbonitrile |
| GT- 0732 2 | 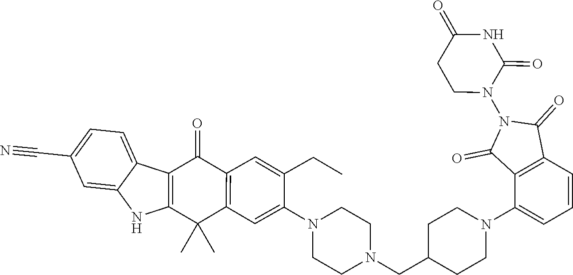 [SMILES: CCc1cc2c(cc1N1CCN(CC3CCN(c4cccc5c4C(=O)N(N4CCC(=O)NC4=O)C5=O)CC3)CC1)C(C)(C)c1[nH]c3cc(C#N)ccc3c1C2=O] | 8-(4-((1-(2-(2,4-dioxotetrahydropyrimidin- 1(2H)-yl)-1,3-dioxoisoindolin-4-yl)piperidin- 4-yl)methyl)piperazin-1-yl)-9-ethyl-6,6- dimethyl-11-oxo-6,11-dihydro-5H- benzo[b]carbazole-3-carbonitrile |
| GT- 0736 2 | 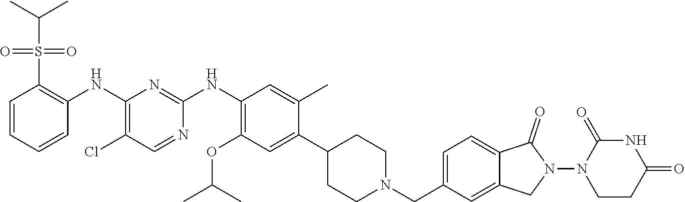 [SMILES: Cc1cc(Nc2ncc(Cl)c(Nc3ccccc3S(=O)(=O)C(C)C)n2)c(OC(C)C)cc1C1CCN(Cc2ccc3c(c2)CN(N2CCC(=O)NC2=O)C3=O)CC1] | 1-(5-((4-(4-((5-chloro-4-((2- (isopropylsulfonyl)phenyl)amino)pyrimidin- 2-yl)amino)-5-isopropoxy-2- methylphenyl)piperidin-1-yl)methyl)-1- oxoisoindolin-2-yl)dihydropyrimidine- 2,4(1H,3H)-dione |
| GT- 0736 3 | 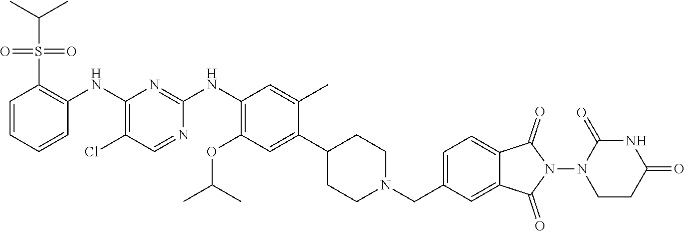 [SMILES: Cc1cc(Nc2ncc(Cl)c(Nc3ccccc3S(=O)(=O)C(C)C)n2)c(OC(C)C)cc1C1CCN(Cc2ccc3c(c2)C(=O)N(N2CCC(=O)NC2=O)C3=O)CC1] | 5-((4-(4-((5-chloro-4-((2- (isopropylsulfonyl)phenyl)amino)pyrimidin- 2-yl)amino)-5-isopropoxy-2- methylphenyl)piperidin-1-yl)methyl)-2-(2,4- dioxotetrahydropyrimidin-1(2H)- yl)isoindoline-1,3-dione |
| GT- 0824 9 | 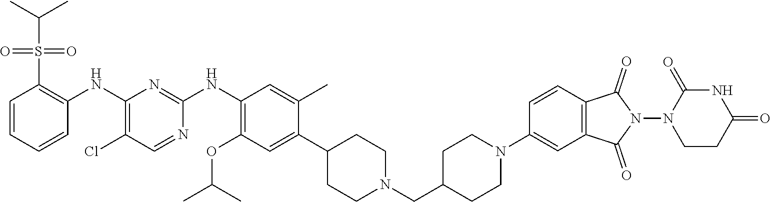 [SMILES: Cc1cc(Nc2ncc(Cl)c(Nc3ccccc3S(=O)(=O)C(C)C)n2)c(OC(C)C)cc1C1CCN(CC2CCN(c3ccc4c(c3)C(=O)N(N3CCC(=O)NC3=O)C4=O)CC2)CC1] | 5-(4-((4-(4-((5-chloro-4-((2- (isopropylsulfonyl)phenyl)amino)pyrimidin- 2-yl)amino)-5-isopropoxy-2- methylphenyl)piperidin-1- yl)methyl)piperidin-1-yl)-2-(2,4- dioxotetrahydropyrimidin-1(2H)- yl)isoindoline-1,3-dione |
| GT- 0825 0 | 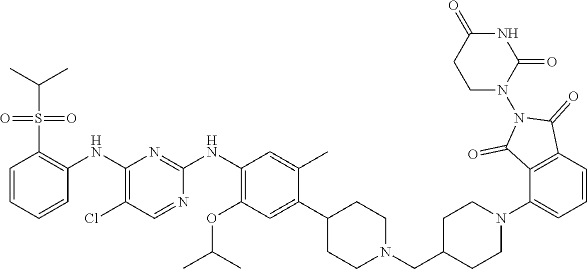 [SMILES: Cc1cc(Nc2ncc(Cl)c(Nc3ccccc3S(=O)(=O)C(C)C)n2)c(OC(C)C)cc1C1CCN(CC2CCN(c3cccc4c3C(=O)N(N3CCC(=O)NC3=O)C4=O)CC2)CC1] | 4-(4-((4-(4-((5-chloro-4-((2- (isopropylsulfonyl)phenyl)amino)pyrimidin- 2-yl)amino)-5-isopropoxy-2- methylphenyl)piperidin-1- yl)methyl)piperidin-1-yl)-2-(2,4- dioxotetrahydropyrimidin-1(2H)- yl)isoindoline-1,3-dione |
| GT- 0825 1 | 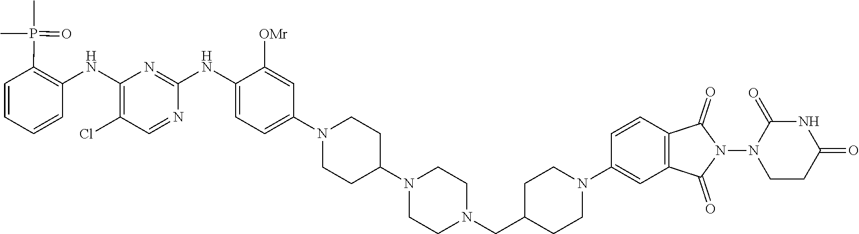 | 5-(4-((4-(1-(4-((5-chloro-4-((2- (dimethylphosphoryl)phenyl)amino)pyrimidin- 2-yl)amino)-3-methoxyphenyl)piperidin-4- yl)piperazin-1-yl)methyl)piperidin-1-yl)-2- (2,4-dioxotetrahydropyrimidin-1(2H)- yl)isoindoline-1,3-dione |
| GT- 0825 2 | 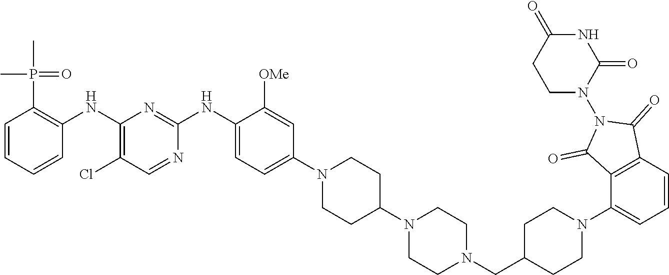 [SMILES: COc1cc(N2CCC(N3CCN(CC4CCN(c5cccc6c5C(=O)N(N5CCC(=O)NC5=O)C6=O)CC4)CC3)CC2)ccc1Nc1ncc(Cl)c(Nc2ccccc2P(C)(C)=O)n1] | 4-(4-((4-(1-(4-((5-chloro-4-((2- (dimethylphosphoryl)phenyl)amino)pyrimidin- 2-yl)amino)-3-methoxyphenyl)piperidin-4- yl)piperazin-1-yl)methyl)piperidin-1-yl)-2- (2,4-dioxotetrahydropyrimidin-1(2H)- yl)isoindoline-1,3-dione |
| GT- 0734 3 | 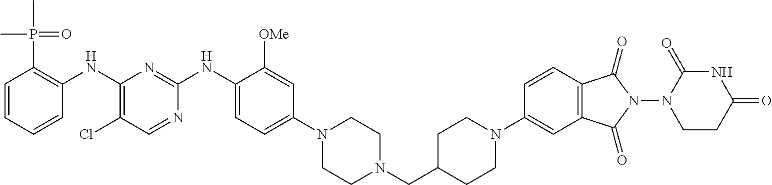 [SMILES: COc1cc(N2CCN(CC3CCN(c4ccc5c(c4)C(=O)N(N4CCC(=O)NC4=O)C5=O)CC3)CC2)ccc1Nc1ncc(Cl)c(Nc2ccccc2P(C)(C)=O)n1] | 5-(4-((4-(4-((5-chloro-4-((2- (dimethylphosphoryl)phenyl)amino)pyrimidin- 2-yl)amino)-3-methoxyphenyl)piperazin-1- yl)methyl)piperidin-1-yl)-2-(2,4- dioxotetrahydropyrimidin-1(2H)- yl)isoindoline-1,3-dione |
| GT- 0734 4 | 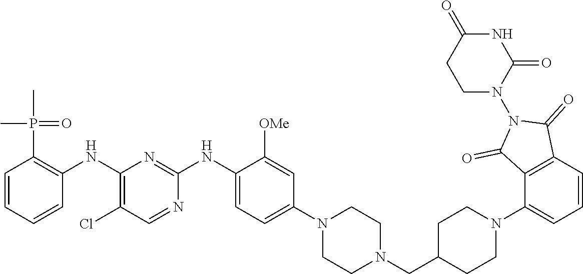 [SMILES: COc1cc(N2CCN(CC3CCN(c4cccc5c4C(=O)N(N4CCC(=O)NC4=O)C5=O)CC3)CC2)ccc1Nc1ncc(Cl)c(Nc2ccccc2P(C)(C)=O)n1] | 4-(4-((4-(4-((5-chloro-4-((2- (dimethylphosphoryl)phenyl)amino)pyrimidin- 2-yl)amino)-3-methoxyphenyl)piperazin-1- yl)methyl)piperidin-1-yl)-2-(2,4- dioxotetrahydropyrimidin-1(2H)- yl)isoindoline-1,3-dione |
| GT- 0825 3 | 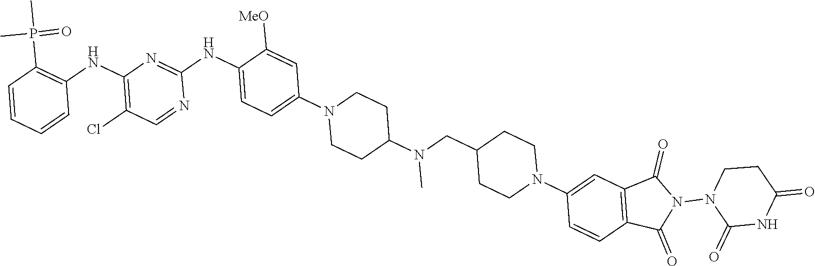 [SMILES: COc1cc(N2CCC(N(C)CC3CCN(c4ccc5c(c4)C(=O)N(N4CCC(=O)NC4=O)C5=O)CC3)CC2)ccc1Nc1ncc(Cl)c(Nc2ccccc2P(C)(C)=O)n1] | 5-(4-(((1-(4-((5-chloro-4-((2- (dimethylphosphoryl)phenyl)amino)pyrimidin- 2-yl)amino)-3-methoxyphenyl)piperidin-4- yl)(methyl)amino)methyl)piperidin-1-yl)-2- (2,4-dioxotetrahydropyrimidin-1(2H)- yl)isoindoline-1,3-dione |
| GT- 0825 4 | 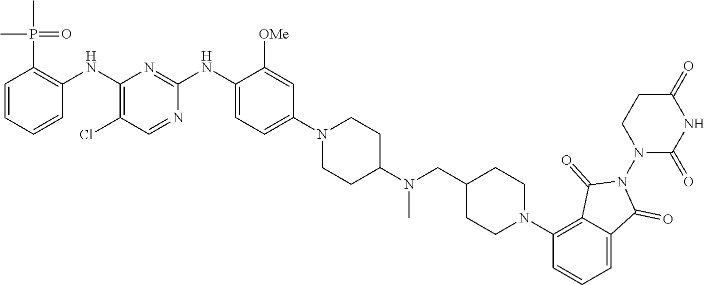 [SMILES: COc1cc(N2CCC(N(C)CC3CCN(c4cccc5c4C(=O)N(N4CCC(=O)NC4=O)C5=O)CC3)CC2)ccc1Nc1ncc(Cl)c(Nc2ccccc2P(C)(C)=O)n1] | 4-(4-(((1-(4-((5-chloro-4-((2- (dimethylphosphoryl)phenyl)amino)pyrimidin- 2-yl)amino)-3-methoxyphenyl)piperidin-4- yl)(methyl)amino)methyl)piperidin-1-yl)-2- (2,4-dioxotetrahydropyrimidin-1(2H)- yl)isoindoline-1,3-dione |
| GT- 0852 6 | 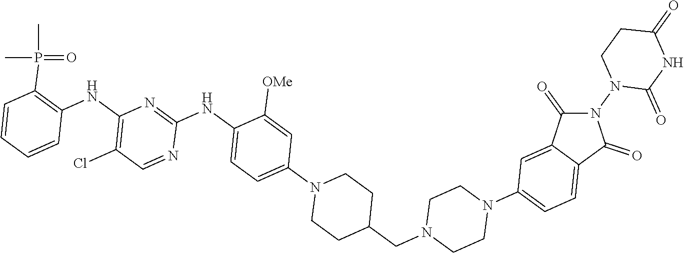 [SMILES: COc1cc(N2CCC(CN3CCN(c4ccc5c(c4)C(=O)N(N4CCC(=O)NC4=O)C5=O)CC3)CC2)ccc1Nc1ncc(Cl)c(Nc2ccccc2P(C)(C)=O)n1] | 5-(4-((1-(4-((5-chloro-4-((2- (dimethylphosphoryl)phenyl)amino)pyrimidin- 2-yl)amino)-3-methoxyphenyl)piperidin-4- yl)methyl)piperazin-1-yl)-2-(2,4- dioxotetrahydropyrimidin-1(2H)- yl)isoindoline-1,3-dione |
| GT- 0852 7 | 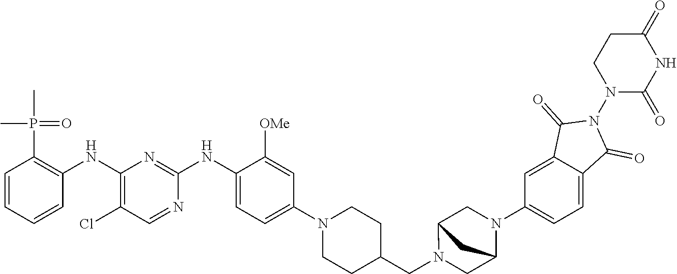 [SMILES: COc1cc(N2CCC(CN3C[C@@H]4C[C@H]3CN4c3ccc4c(c3)C(=O)N(N3CCC(=O)NC3=O)C4=O)CC2)ccc1Nc1ncc(Cl)c(Nc2ccccc2P(C)(C)=O)n1] | 5-((1S,4S)-5-((1-(4-((5-chloro-4-((2- (dimethylphosphoryl)phenyl)amino)pyrimidin- 2-yl)amino)-3-methoxyphenyl)piperidin-4- yl)methyl)-2,5-diazabicyclo[2.2.1]heptan-2- yl)-2-(2,4-dioxotetrahydropyrimidin-1(2H)- yl)isoindoline-1,3-dione |
| GT- 0852 8 | 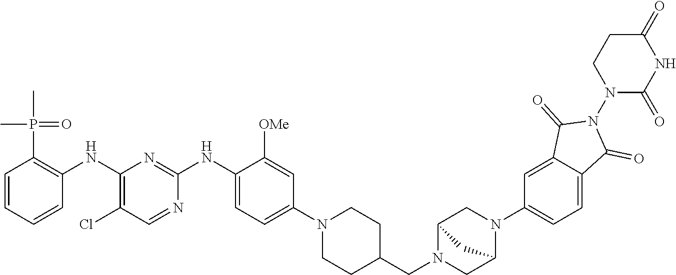 [SMILES: COc1cc(N2CCC(CN3C[C@H]4C[C@@H]3CN4c3ccc4c(c3)C(=O)N(N3CCC(=O)NC3=O)C4=O)CC2)ccc1Nc1ncc(Cl)c(Nc2ccccc2P(C)(C)=O)n1] | 5-((1R,4R)-5-((1-(4-((5-chloro-4-((2- (dimethylphosphoryl)phenyl)amino)pyrimidin- 2-yl)amino)-3-methoxyphenyl)piperidin-4- yl)methyl)-2,5-diazabicyclo[2.2.1]heptan-2- yl)-2-(2,4-dioxotetrahydropyrimidin-1(2H)- yl)isoindoline-1,3-dione |
| GT- 0852 9 | 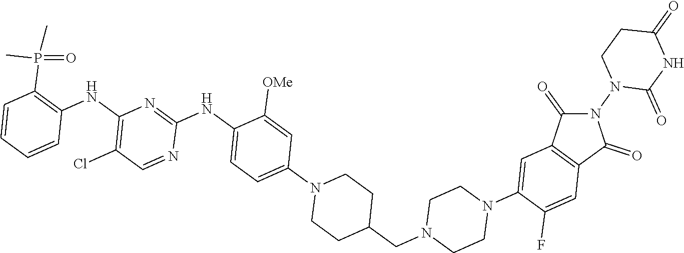 [SMILES: COc1cc(N2CCC(CN3CCN(c4cc5c(cc4F)C(=O)N(N4CCC(=O)NC4=O)C5=O)CC3)CC2)ccc1Nc1ncc(Cl)c(Nc2ccccc2P(C)(C)=O)n1] | 5-(4-((1-(4-((5-chloro-4-((2- (dimethylphosphoryl)phenyl)amino)pyrimidin- 2-yl)amino)-3-methoxyphenyl)piperidin-4- yl)methyl)piperazin-1-yl)-2-(2,4- dioxotetrahydropyrimidin-1(2H)-yl)-6- fluoroisoindoline-1,3-dione |
| GT- 0853 0 | 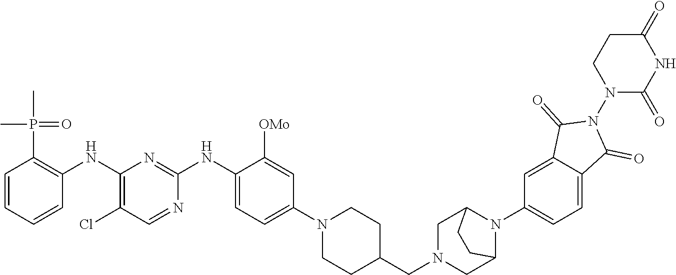 | 5-(3-((1-(4-((5-chloro-4-((2- (dimethylphosphoryl)phenyl)amino)pyrimidin- 2-yl)amino)-3-methoxyphenyl)piperidin-4- yl)methyl)-3,8-diazabicyclo[3.2.1]octan-8- yl)-2-(2,4-dioxotetrahydropyrimidin-1(2H)- yl)isoindoline-1,3-dione |
| GT- 0746 8 | 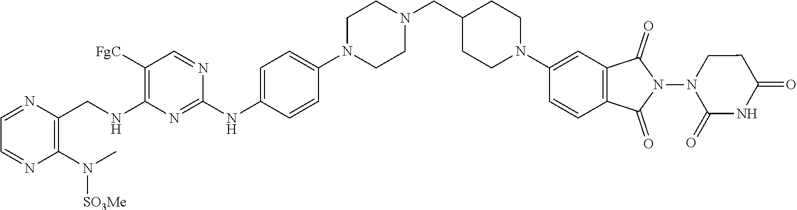 | N-(3-(((2-((4-(4-((1-(2-(2,4- dioxotetrahydropyrimidin-1(2H)-yl)-1,3- dioxoisoindolin-5-yl)piperidin-4- yl)methyl)piperazin-1-yl)phenyl)amino)-5- (trifluoromethyl)pyrimidin-4- yl)amino)methyl)pyrazin-2-yl)-N- methylmethanesulfonamide |
| GT- 0746 9 | 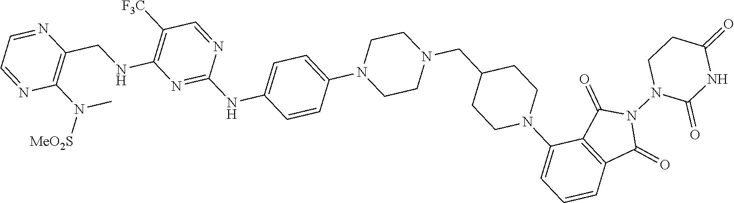 [SMILES: CN(c1nccnc1CNc1nc(Nc2ccc(N3CCN(CC4CCN(c5cccc6c5C(=O)N(N5CCC(=O)NC5=O)C6=O)CC4)CC3)cc2)ncc1C(F)(F)F)S(C)(=O)=O] | N-(3-(((2-((4-(4-((1-(2-(2,4- dioxotetrahydropyrimidin-1(2H)-yl)-1,3- dioxoisoindolin-4-yl)piperidin-4- yl)methyl)piperazin-1-yl)phenyl)amino)-5- (trifluoromethyl)pyrimidin-4- yl)amino)methyl)pyrazin-2-yl)-N- methylmethanesulfonamide |
| GT- 0824 5 | 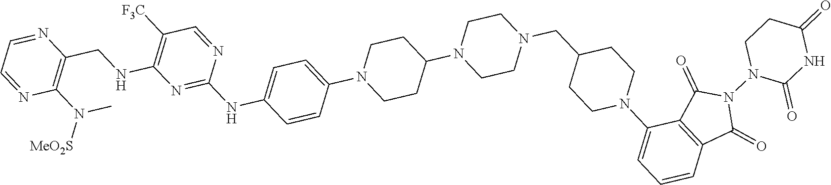 [SMILES: CN(c1nccnc1CNc1nc(Nc2ccc(N3CCC(N4CCN(CC5CCN(c6cccc7c6C(=O)N(N6CCC(=O)NC6=O)C7=O)CC5)CC4)CC3)cc2)ncc1C(F)(F)F)S(C)(=O)=O] | N-(3-(((2-((4-(4-(4-((1-(2-(2,4- dioxotetrahydropyrimidin-1(2H)-yl)-1,3- dioxoisoindolin-4-yl)piperidin-4- yl)methyl)piperazin-1-yl)piperidin-1- yl)phenyl)amino)-5- (trifluoromethyl)pyrimidin-4- yl)amino)methyl)pyrazin-2-yl)-N- methylmethanesulfonamide |
| GT- 0747 0 | 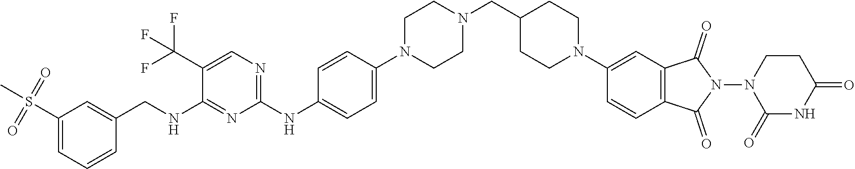 [SMILES: CS(=O)(=O)c1cccc(CNc2nc(Nc3ccc(N4CCN(CC5CCN(c6ccc7c(c6)C(=O)N(N6CCC(=O)NC6=O)C7=O)CC5)CC4)cc3)ncc2C(F)(F)F)c1] | 2-(2,4-dioxotetrahydropyrimidin-1(2H)-yl)-5- (4-((4-(4-((4-((3- (methylsulfonyl)benzyl)amino)-5- (trifluoromethyl)pyrimidin-2- yl)amino)phenyl)piperazin-1- yl)methyl)piperidin-1-yl)isoindoline-1,3- dione |
| GT- 0747 1 | 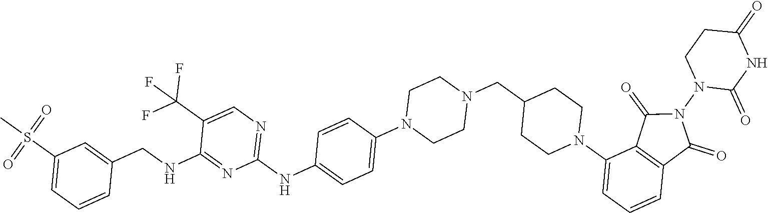 [SMILES: CS(=O)(=O)c1cccc(CNc2nc(Nc3ccc(N4CCN(CC5CCN(c6cccc7c6C(=O)N(N6CCC(=O)NC6=O)C7=O)CC5)CC4)cc3)ncc2C(F)(F)F)c1] | 2-(2,4-dioxotetrahydropyrimidin-1(2H)-yl)-4- (4-((4-(4-((4-((3- (methylsulfonyl)benzyl)amino)-5- (trifluoromethyl)pyrimidin-2- yl)amino)phenyl)piperazin-1- yl)methyl)piperidin-1-yl)isoindoline-1,3- dione |
| GT- 0824 6 | 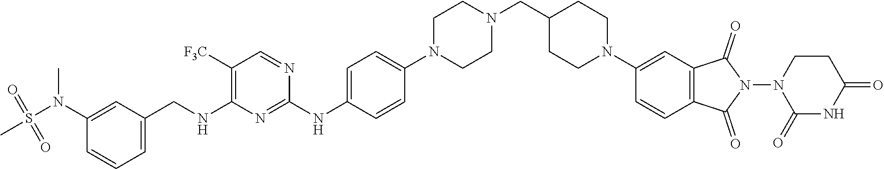 [SMILES: CN(c1cccc(CNc2nc(Nc3ccc(N4CCN(CC5CCN(c6ccc7c(c6)C(=O)N(N6CCC(=O)NC6=O)C7=O)CC5)CC4)cc3)ncc2C(F)(F)F)c1)S(C)(=O)=O] | N-(3-(((2-((4-(4-((1-(2-(2,4- dioxotetrahydropyrimidin-1(2H)-yl)-1,3- dioxoisoindolin-5-yl)piperidin-4- yl)methyl)piperazin-1-yl)phenyl)amino)-5- (trifluoromethyl)pyrimidin-4- yl)amino)methyl)phenyl)-N- methylmethanesulfonamide |
| GT- 0824 7 | 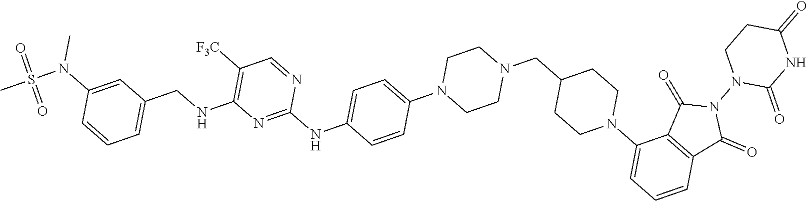 [SMILES: CN(c1cccc(CNc2nc(Nc3ccc(N4CCN(CC5CCN(c6cccc7c6C(=O)N(N6CCC(=O)NC6=O)C7=O)CC5)CC4)cc3)ncc2C(F)(F)F)c1)S(C)(=O)=O] | N-(3-(((2-((4-(4-((1-(2-(2,4- dioxotetrahydropyrimidin-1(2H)-yl)-1,3- dioxoisoindolin-4-yl)piperidin-4- yl)methyl)piperazin-1-yl)phenyl)amino)-5- (trifluoromethyl)pyrimidin-4- yl)amino)methyl)phenyl)-N- methylmethanesulfonamide |
| GT- 0824 8 | 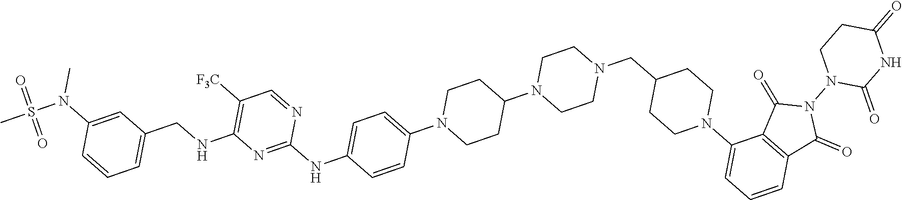 [SMILES: CN(c1cccc(CNc2nc(Nc3ccc(N4CCC(N5CCN(CC6CCN(c7cccc8c7C(=O)N(N7CCC(=O)NC7=O)C8=O)CC6)CC5)CC4)cc3)ncc2C(F)(F)F)c1)S(C)(=O)=O] | N-(3-(((2-((4-(4-(4-((1-(2-(2,4- dioxotetrahydropyrimidin-1(2H)-yl)-1,3- dioxoisoindolin-4-yl)piperidin-4- yl)methyl)piperazin-1-yl)piperidin-1- yl)phenyl)amino)-5- (trifluoromethyl)pyrimidin-4- yl)amino)methyl)phenyl)-N- methylmethanesulfonamide |
| GT- 0766 5 | 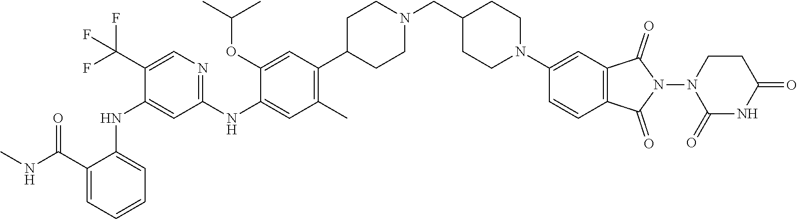 [SMILES: CNC(=O)c1ccccc1Nc1cc(Nc2cc(C)c(C3CCN(CC4CCN(c5ccc6c(c5)C(=O)N(N5CCC(=O)NC5=O)C6=O)CC4)CC3)cc2OC(C)C)ncc1C(F)(F)F] | 2-((2-((4-(1-((1-(2-(2,4- dioxotetrahydropyrimidin-1(2H)-yl)-1,3- dioxoisoindolin-5-yl)piperidin-4- yl)methyl)piperidin-4-yl)-2-isopropoxy-5- methylphenyl)amino)-5- (trifluoromethyl)pyridin-4-yl)amino)-N- methylbenzamide |
| GT- 0766 6 | 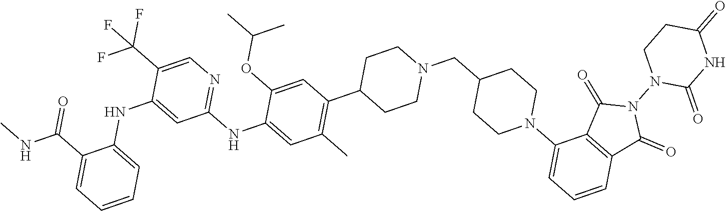 [SMILES: CNC(=O)c1ccccc1Nc1cc(Nc2cc(C)c(C3CCN(CC4CCN(c5cccc6c5C(=O)N(N5CCC(=O)NC5=O)C6=O)CC4)CC3)cc2OC(C)C)ncc1C(F)(F)F] | 2-((2-((4-(1-((1-(2-(2,4- dioxotetrahydropyrimidin-1(2H)-yl)-1,3- dioxoisoindolin-4-yl)piperidin-4- yl)methyl)piperidin-4-yl)-2-isopropoxy-5- methylphenyl)amino)-5- (trifluoromethyl)pyridin-4-yl)amino)-N- methylbenzamide |
| GT- 0581 3 | 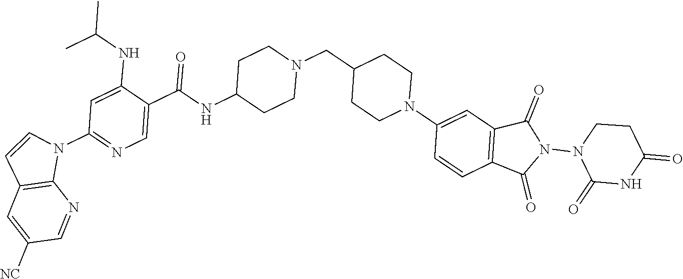 [SMILES: CC(C)Nc1cc(-n2ccc3cc(C#N)cnc32)ncc1C(=O)NC1CCN(CC2CCN(c3ccc4c(c3)C(=O)N(N3CCC(=O)NC3=O)C4=O)CC2)CC1] | 6-(5-cyano-1H-pyrrolo[2,3-b]pyridin-1-yl)-N- (1 -((1-(2-(2,4-dioxotetrahydropyrimidin- 1(2H)-yl)-1,3-dioxoisoindolin-5-yl)piperidin- 4-yl)methyl)piperidin-4-yl)-4- (isopropylamino)nicotinamide |
| GT- 0581 4 | 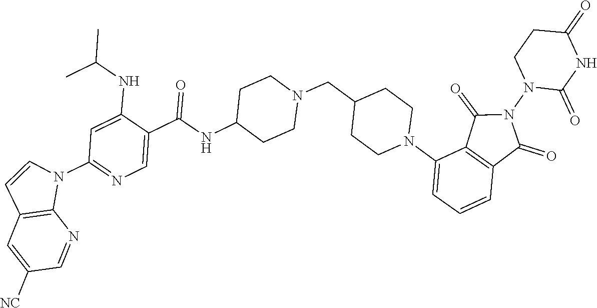 [SMILES: CC(C)Nc1cc(-n2ccc3cc(C#N)cnc32)ncc1C(=O)NC1CCN(CC2CCN(c3cccc4c3C(=O)N(N3CCC(=O)NC3=O)C4=O)CC2)CC1] | 6-(5-cyano-1H-pyrrolo[2,3-b]pyridin-1-yl)-N- (1-((1-(2-(2,4-dioxotetrahydropyrimidin- 1(2H)-yl)-1,3-dioxoisoindolin-4-yl)piperidin- 4-yl)methyl)piperidin-4-yl)-4- (isopropylamino)nicotinamide |
| GT- 0581 5 | 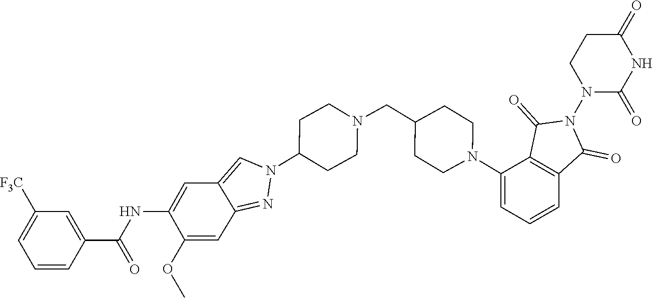 [SMILES: COc1cc2nn(C3CCN(CC4CCN(c5cccc6c5C(=O)N(N5CCC(=O)NC5=O)C6=O)CC4)CC3)cc2cc1NC(=O)c1cccc(C(F)(F)F)c1] | N-(2-(1-((1-(2-(2,4- dioxotetrahydropyrimidin-1(2H)-yl)-1,3- dioxoisoindolin-4-yl)piperidin-4- yl)methyl)piperidin-4-yl)-6-methoxy-2H- indazol-5-yl)-3-(trifluoromethyl)benzamide |
| GT- 0581 7 | 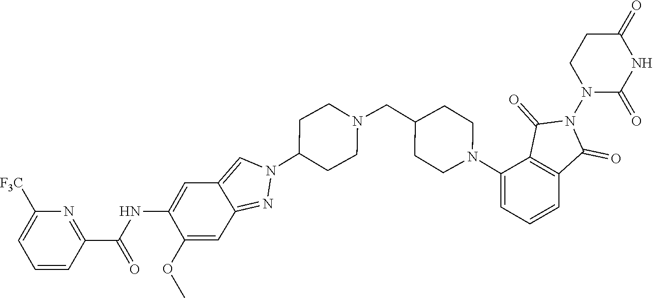 [SMILES: COc1cc2nn(C3CCN(CC4CCN(c5cccc6c5C(=O)N(N5CCC(=O)NC5=O)C6=O)CC4)CC3)cc2cc1NC(=O)c1cccc(C(F)(F)F)n1] | N-(2-(1-((1-(2-(2,4- dioxotetrahydropyrimidin-1(2H)-yl)-1,3- dioxoisoindolin-4-yl)piperidin-4- yl)methyl)piperidin-4-yl)-6-methoxy-2H- indazol-5-yl)-6-(trifluoromethyl)picolinamide |
| GT- 0581 8 | 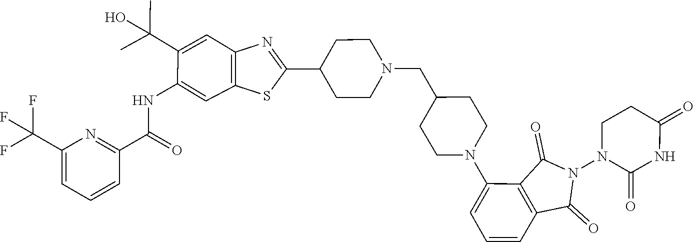 [SMILES: CC(C)(O)c1cc2nc(C3CCN(CC4CCN(c5cccc6c5C(=O)N(N5CCC(=O)NC5=O)C6=O)CC4)CC3)sc2cc1NC(=O)c1cccc(C(F)(F)F)n1] | N-(2-(1-((1-(2-(2,4- dioxotetrahydropyrimidin-1(2H)-yl)-1,3- dioxoisoindolin-4-yl)piperidin-4- yl)methyl)piperidin-4-yl)-5-(2- hydroxypropan-2-yl)benzo[d]thiazol-6-yl)-6- (trifluoromethyl)picolinamide |
| GT- 0581 9 | 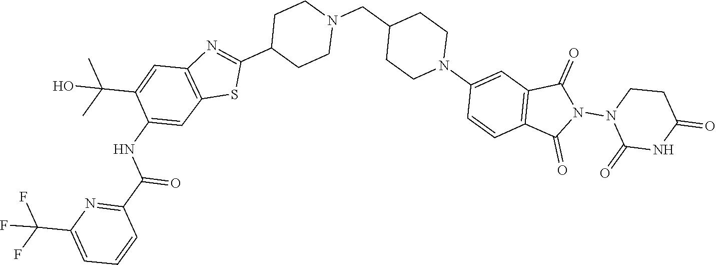 [SMILES: CC(C)(O)c1cc2nc(C3CCN(CC4CCN(c5ccc6c(c5)C(=O)N(N5CCC(=O)NC5=O)C6=O)CC4)CC3)sc2cc1NC(=O)c1cccc(C(F)(F)F)n1] | N-(2-(1-((1-(2-(2,4- dioxotetrahydropyrimidin-1(2H)-yl)-1,3- dioxoisoindolin-5-yl)piperidin-4- yl)methyl)piperidin-4-yl)-5-(2- hydroxypropan-2-yl)benzo[d]thiazol-6-yl)-6- (trifluoromethyl)picolinamide |
| GT- 0575 9 | 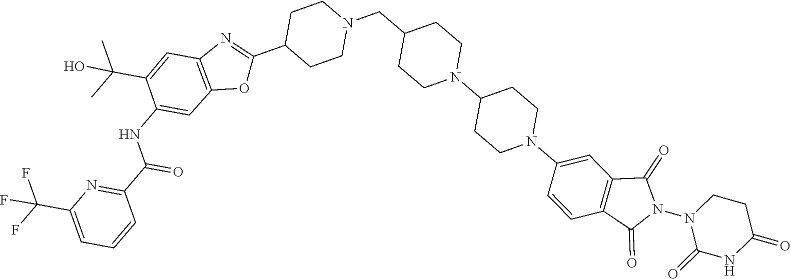 [SMILES: CC(C)(O)c1cc2nc(C3CCN(CC4CCN(C5CCN(c6ccc7c(c6)C(=O)N(N6CCC(=O)NC6=O)C7=O)CC5)CC4)CC3)oc2cc1NC(=O)c1cccc(C(F)(F)F)n1] | N-(2-(1-((1′-(2-(2,4- dioxotetrahydropyrimidin-1(2H)-yl)-1,3- dioxoisoindolin-5-yl)-[1,4′-bipiperidin]-4- yl)methyl)piperidin-4-yl)-5-(2- hydroxypropan-2-yl)benzo[d]oxazol-6-yl)-6- (trifluoromethyl)picolinamide |
| GT- 0576 0 | 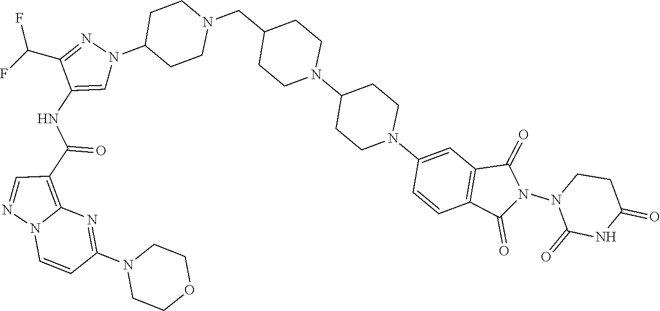 [SMILES: O=C1CCN(N2C(=O)c3ccc(N4CCC(N5CCC(CN6CCC(n7cc(NC(=O)c8cnn9ccc(N%10CCOCC%10)nc89)c(C(F)F)n7)CC6)CC5)CC4)cc3C2=O)C(=O)N1] | N-(3-(difluoromethyl)-1-(1-((1′-(2-(2,4- dioxotetrahydropyrimidin-1(2H)-yl)-1,3- dioxoisoindolin-5-yl)-[1,4′-bipiperidin]-4- yl)methyl)piperidin-4-yl)-1H-pyrazol-4-yl)-5- morpholinopyrazolo[1,5-a]pyrimidine-3- carboxamide |
| GT- 0581 6 | 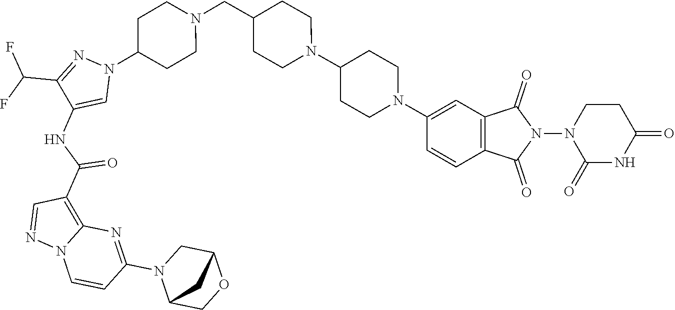 [SMILES: O=C1CCN(N2C(=O)c3ccc(N4CCC(N5CCC(CN6CCC(n7cc(NC(=O)c8cnn9ccc(N%10C[C@H]%11C[C@@H]%10CO%11)nc89)c(C(F)F)n7)CC6)CC5)CC4)cc3C2=O)C(=O)N1] | 5-((1R,4R)-2-oxa-5-azabicyclo[2.2.1]heptan- 5-yl)-N-(3-(difluoromethyl)-1-(1-((1′-(2-(2,4- dioxotetrahydropyrimidin-1(2H)-yl)-1,3- dioxoisoindolin-5-yl)-[1,4′-bipiperidin]-4- yl)methyl)piperidin-4-yl)-1H-pyrazol-4- yl)pyrazolo[1,5-a]pyrimidine-3-carboxamide |
| GT- 0580 5 | 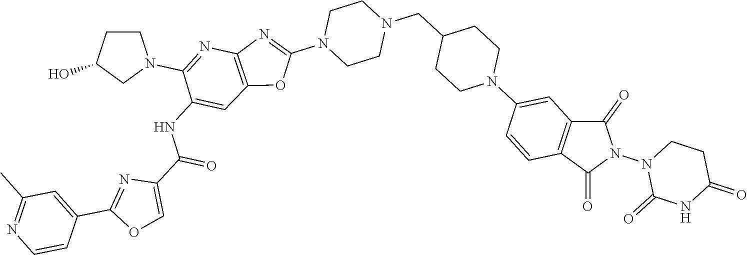 [SMILES: Cc1cc(-c2nc(C(=O)Nc3cc4oc(N5CCN(CC6CCN(c7ccc8c(c7)C(=O)N(N7CCC(=O)NC7=O)C8=O)CC6)CC5)nc4nc3N3CC[C@@H](O)C3)co2)ccn1] | (R)-N-(2-(4-((1-(2-(2,4- dioxotetrahydropyrimidin-1(2H)-yl)-1,3- dioxoisoindolin-5-yl)piperidin-4- yl)methyl)piperazin-1-yl)-5-(3- hydroxypyrrolidin-1-yl)oxazolo[4,5- b]pyridin-6-yl)-2-(2-methylpyridin-4- yl)oxazole-4-carboxamide |
| GT- 0580 6 | 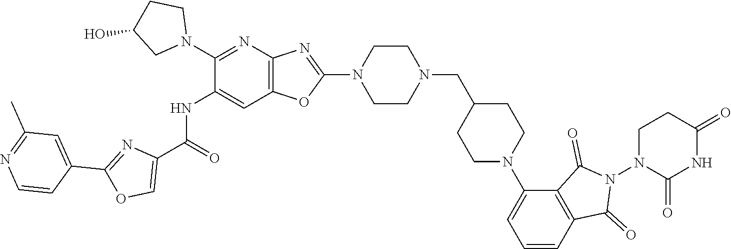 [SMILES: Cc1cc(-c2nc(C(=O)Nc3cc4oc(N5CCN(CC6CCN(c7cccc8c7C(=O)N(N7CCC(=O)NC7=O)C8=O)CC6)CC5)nc4nc3N3CC[C@@H](O)C3)co2)ccn1] | (R)-N-(2-(4-((1-(2-(2,4- dioxotetrahydropyrimidin-1(2H)-yl)-1,3- dioxoisoindolin-4-yl)piperidin-4- yl)methyl)piperazin-1-yl)-5-(3- hydroxypyrrolidin-1-yl)oxazolo[4,5- b]pyridin-6-yl)-2-(2-methylpyridin-4- yl)oxazole-4-carboxamide |
| GT- 0616 8 | 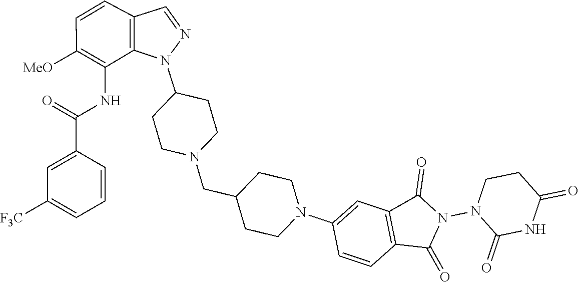 [SMILES: COc1ccc2cnn(C3CCN(CC4CCN(c5ccc6c(c5)C(=O)N(N5CCC(=O)NC5=O)C6=O)CC4)CC3)c2c1NC(=O)c1cccc(C(F)(F)F)c1] | N-(1-(1-((1-(2-(2,4- dioxotetrahydropyrimidin-1(2H)-yl)-1,3- dioxoisoindolin-5-yl)piperidin-4- yl)methyl)piperidin-4-yl)-6-methoxy-1H- indazol-7-yl)-3-(trifluoromethyl)benzamide |
| GT- 0864 7 | 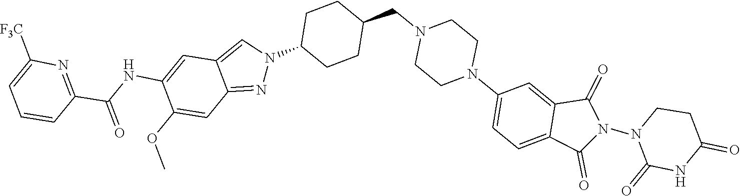 [SMILES: COc1cc2nn([C@H]3CC[C@H](CN4CCN(c5ccc6c(c5)C(=O)N(N5CCC(=O)NC5=O)C6=O)CC4)CC3)cc2cc1NC(=O)c1cccc(C(F)(F)F)n1] | N-(2-((1r,4r)-4-((4-(2-(2,4- dioxotetrahydropyrimidin-1(2H)-yl)-1,3- dioxoisoindolin-5-yl)piperazin-1- yl)methyl)cyclohexyl)-6-methoxy-2H- indazol-5-yl)-6-(trifluoromethyl)picolinamide |
| GT- 0864 8 | 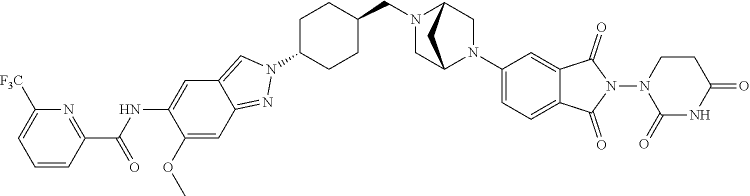 | N-(2-((1S,4r)-4-(((1S,4S)-5-(2-(2,4- dioxotetrahydropyrimidin-1(2H)-yl)-1,3- dioxoisoindolin-5-yl)-2,5- diazabicyclo[2.2.1]heptan-2- yl)methyl)cyclohexyl)-6-methoxy-2H- indazol-5-yl)-6-(trifluoromethyl)picolinamide |
| GT- 0865 0 | 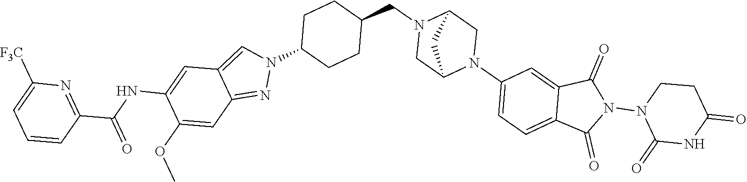 | N-(2-((1R,4r)-4-(((1R,4R)-5-(2-(2,4- dioxotetrahydropyrimidin-1(2H)-yl)-1,3- dioxoisoindolin-5-yl)-2,5- yl)methyl)cyclohexyl)-6-methoxy-2H- indazol-5-yl)-6-(trifluoromethyl)picolinamide |
| GT- 0865 1 | 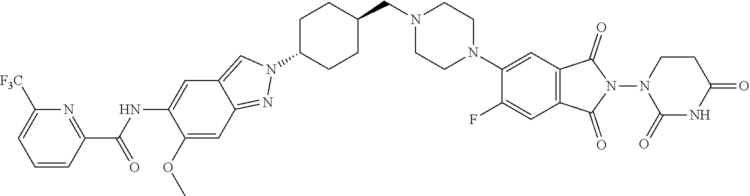 | N-(2-((1r,4r)-4-((4-(2-(2,4- dioxotetrahydropyrimidin-1(2H)-yl)-6-fluoro- 1,3-dioxoisoindolin-5-yl)piperazin-1- yl)methyl)cyclohexyl)-6-methoxy-2H- indazol-5-yl)-6-(trifluoromethyl)picolinamide |
| GT- 0865 2 | 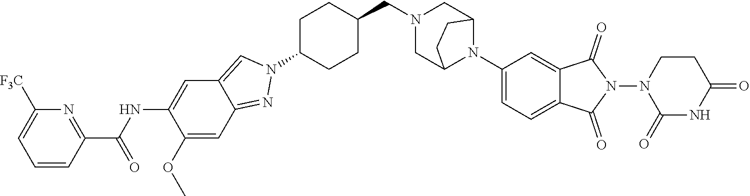 | N-(2-((1r,4r)-4-((8-(2-(2,4- dioxotetrahydropyrimidin-1(2H)-yl)-1,3- dioxoisoindolin-5-yl)-3,8- diazabicyclo[3.2.1]octan-3- yl)methyl)cyclohexyl)-6-methoxy-2H- indazol-5-yl)-6-(trifluoromethyl)picolinamide |
| GT- 0865 3 | 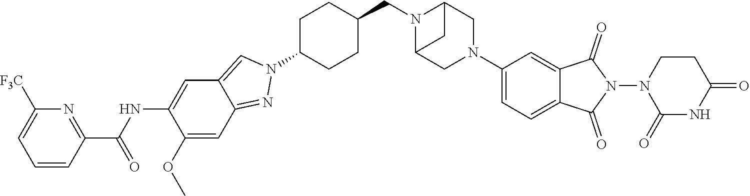 | N-(2-((1r,4r)-4-((3-(2-(2,4- dioxotetrahydropyrimidin-1(2H)-yl)-1,3- dioxoisoindolin-5-yl)-3,6- diazabicyclo[3.1.1]heptan-6- yl)methyl)cyclohexyl)-6-methoxy-2H- indazol-5-yl)-6-(trifluoromethyl)picolinamide |
| GT- 0866 1 | 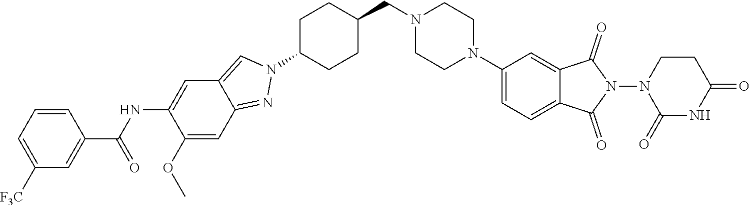 [SMILES: COc1cc2nn([C@H]3CC[C@H](CN4CCN(c5ccc6c(c5)C(=O)N(N5CCC(=O)NC5=O)C6=O)CC4)CC3)cc2cc1NC(=O)c1cccc(C(F)(F)F)c1] | N-(2-((1r,4r)-4-((4-(2-(2,4- dioxotetrahydropyrimidin-1(2H)-yl)-1,3- dioxoisoindolin-5-yl)piperazin-1- yl)methyl)cyclohexyl)-6-methoxy-2H- indazol-5-yl)-3-(trifluoromethyl)benzamide |
| GT- 0866 2 | 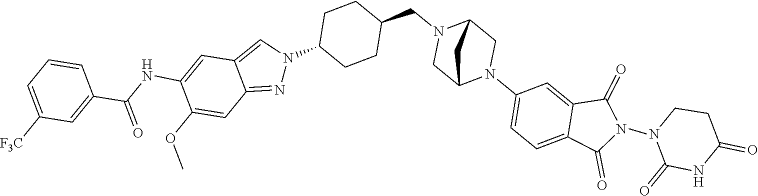 [SMILES: COc1cc2nn([C@H]3CC[C@H](CN4C[C@@H]5C[C@H]4CN5c4ccc5c(c4)C(=O)N(N4CCC(=O)NC4=O)C5=O)CC3)cc2cc1NC(=O)c1cccc(C(F)(F)F)c1] | N-(2-((1S,4r)-4-(((1S,4S)-5-(2-(2,4- dioxotetrahydropyrimidin-1(2H)-yl)-1,3- dioxoisoindolin-5-yl)-2,5- diazabicyclo[2.2.1]heptan-2- yl)methyl)cyclohexyl)-6-methoxy-2H- indazol-5-yl)-3-(trifluoromethyl)benzamide |
| GT- 0866 3 | 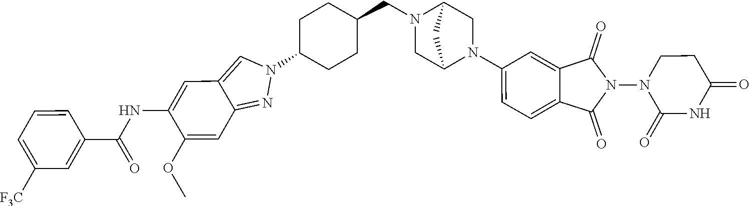 [SMILES: COc1cc2nn([C@H]3CC[C@H](CN4C[C@H]5C[C@@H]4CN5c4ccc5c(c4)C(=O)N(N4CCC(=O)NC4=O)C5=O)CC3)cc2cc1NC(=O)c1cccc(C(F)(F)F)c1] | N-(2-((1R,4r)-4-(((1R,4R)-5-(2-(2,4- dioxotetrahydropyrimidin-1(2H)-yl)-1,3- dioxoisoindolin-5-yl)-2,5- diazabicyclo[2.2.1]heptan-2- yl)methyl)cyclohexyl)-6-methoxy-2H- indazol-5-yl)-3-(trifluoromethyl)benzamide |
| GT- 0866 4 | 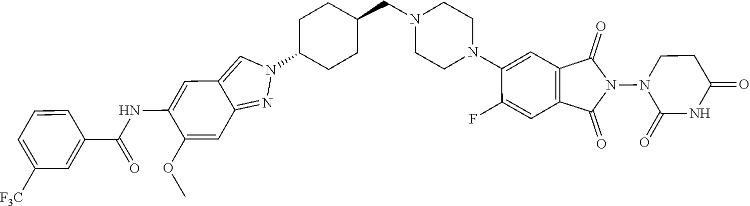 [SMILES: COc1cc2nn([C@H]3CC[C@H](CN4CCN(c5cc6c(cc5F)C(=O)N(N5CCC(=O)NC5=O)C6=O)CC4)CC3)cc2cc1NC(=O)c1cccc(C(F)(F)F)c1] | N-(2-((1r,4r)-4-((4-(2-(2,4- dioxotetrahydropyrimidin-1(2H)-yl)-6-fluoro- 1,3-dioxoisoindolin-5-yl)piperazin-1- yl)methyl)cyclohexyl)-6-methoxy-2H- indazol-5-yl)-3-(trifluoromethyl)benzamide |
| GT- 0866 5 | 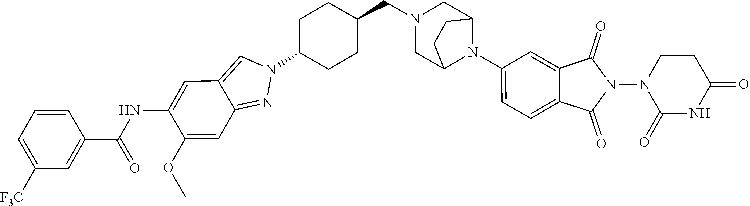 [SMILES: COc1cc2nn([C@H]3CC[C@H](CN4CC5CCC(C4)N5c4ccc5c(c4)C(=O)N(N4CCC(=O)NC4=O)C5=O)CC3)cc2cc1NC(=O)c1cccc(C(F)(F)F)c1] | N-(2-((1r,4r)-4-((8-(2-(2,4- dioxotetrahydropyrimidin-1(2H)-yl)-1,3- dioxoisoindolin-5-yl)-3,8- diazabicyclo[3.2.1]octan-3- yl)methyl)cyclohexyl)-6-methoxy-2H- indazol-5-yl)-3-(trifluoromethyl)benzamide |
| GT- 0866 6 | 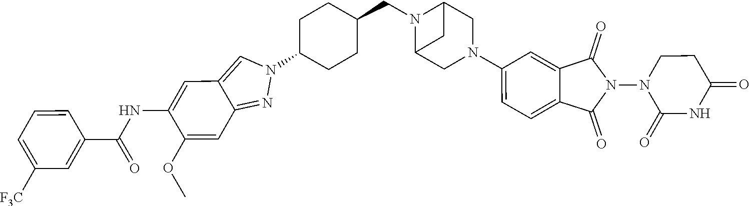 [SMILES: COc1cc2nn([C@H]3CC[C@H](CN4C5CC4CN(c4ccc6c(c4)C(=O)N(N4CCC(=O)NC4=O)C6=O)C5)CC3)cc2cc1NC(=O)c1cccc(C(F)(F)F)c1] | N-(2-((1r,4r)-4-((3-(2-(2,4- dioxotetrahydropyrimidin-1(2H)-yl)-1,3- dioxoisoindolin-5-yl)-3,6- diazabicyclo[3.1.1]heptan-6- yl)methyl)cyclohexyl)-6-methoxy-2H- indazol-5-yl)-3-(trifluoromethyl)benzamide |
| GT- 0865 4 | 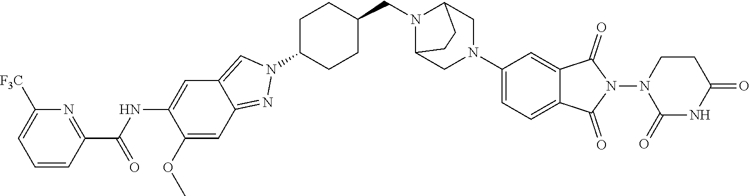 | N-(2-((1r,4r)-4-((3-(2-(2,4- dioxotetrahydropyrimidin-1(2H)-yl)-1,3- dioxoisoindolin-5-yl)-3,8- diazabicyclo[3.2.1]octan-8- yl)methyl)cyclohexyl)-6-methoxy-2H- indazol-5-yl)-6-(trifluoromethyl)picolinamide |
| GT- 0865 5 | 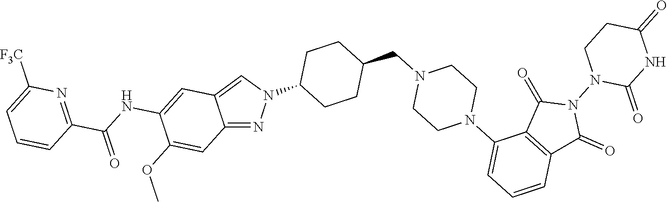 [SMILES: COc1cc2nn([C@H]3CC[C@H](CN4CCN(c5cccc6c5C(=O)N(N5CCC(=O)NC5=O)C6=O)CC4)CC3)cc2cc1NC(=O)c1cccc(C(F)(F)F)n1] | N-(2-((1r,4r)-4-((4-(2-(2,4- dioxotetrahydropyrimidin-1(2H)-yl)-1,3- dioxoisoindolin-4-yl)piperazin-1- yl)methyl)cyclohexyl)-6-methoxy-2H- indazol-5-yl)-6-(trifluoromethyl)picolinamide |
| GT- 0865 6 | 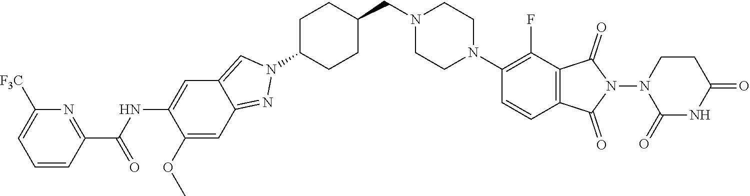 | N-(2-((1r,4r)-4-((4-(2-(2,4- dioxotetrahydropyrimidin-1(2H)-yl)-4-fluoro- 1,3-dioxoisoindolin-5-yl)piperazin-1- yl)methyl)cyclohexyl)-6-methoxy-2H- indazol-5-yl)-6-(trifluoromethyl)picolinamide |
| GT- 0865 7 | 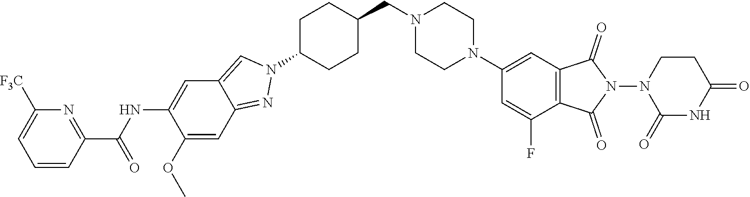 | N-(2-((1r,4r)-4-((4-(2-(2,4- dioxotetrahydropyrimidin-1(2H)-yl)-7-fluoro- 1,3-dioxoisoindolin-5-yl)piperazin-1- yl)methyl)cyclohexyl)-6-methoxy-2H- indazol-5-yl)-6-(trifluoromethyl)picolinamide |
| GT- 0865 8 | 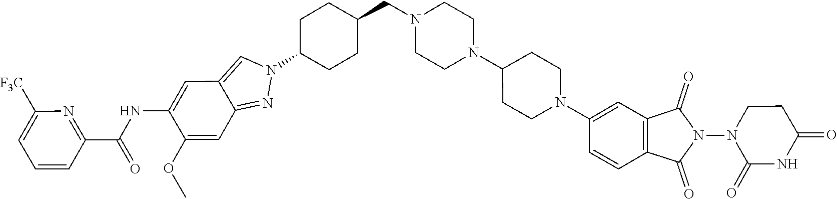 [SMILES: COc1cc2nn([C@H]3CC[C@H](CN4CCN(C5CCN(c6ccc7c(c6)C(=O)N(N6CCC(=O)NC6=O)C7=O)CC5)CC4)CC3)cc2cc1NC(=O)c1cccc(C(F)(F)F)n1] | N-(2-((1r,4r)-4-((4-(1-(2-(2,4- dioxotetrahydropyrimidin-1(2H)-yl)-1,3- dioxoisoindolin-5-yl)piperidin-4-yl)piperazin- 1-yl)methyl)cyclohexyl)-6-methoxy-2H- indazol-5-yl)-6-(trifluoromethyl)picolinamide |
| GT- 0865 9 | 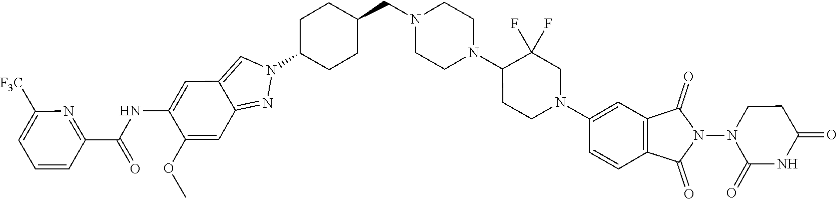 [SMILES: COc1cc2nn([C@H]3CC[C@H](CN4CCN(C5CCN(c6ccc7c(c6)C(=O)N(N6CCC(=O)NC6=O)C7=O)CC5(F)F)CC4)CC3)cc2cc1NC(=O)c1cccc(C(F)(F)F)n1] | N-(2-((1r,4r)-4-((4-(1-(2-(2,4- dioxotetrahydropyrimidin-1(2H)-yl)-1,3- dioxoisoindolin-5-yl)-3,3-difluoropiperidin-4- yl)piperazin-1-yl)methyl)cyclohexyl)-6- methoxy-2H-indazol-5-yl)-6- (trifluoromethyl)picolinamide |
| GT- 0866 0 | 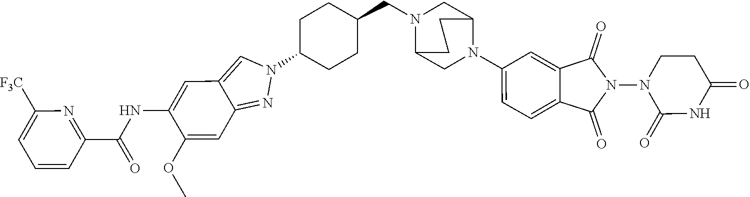 [SMILES: COc1cc2nn([C@H]3CC[C@H](CN4CC5CCC4CN5c4ccc5c(c4)C(=O)N(N4CCC(=O)NC4=O)C5=O)CC3)cc2cc1NC(=O)c1cccc(C(F)(F)F)n1] | N-(2-((1r,4r)-4-((5-(2-(2,4- dioxotetrahydropyrimidin-1(2H)-yl)-1,3- dioxoisoindolin-5-yl)-2,5- diazabicyclo[2.2.2]octan-2- yl)methyl)cyclohexyl)-6-methoxy-2H- indazol-5-yl)-6-(trifluoromethyl)picolinamide |
| GT- 0866 7 | 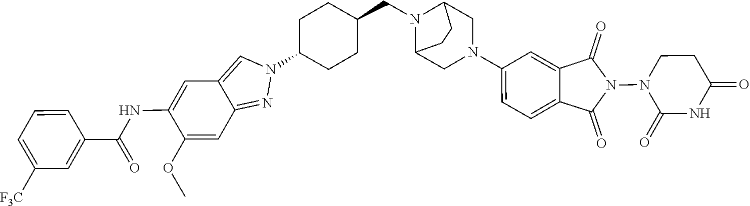 [SMILES: COc1cc2nn([C@H]3CC[C@H](CN4C5CCC4CN(c4ccc6c(c4)C(=O)N(N4CCC(=O)NC4=O)C6=O)C5)CC3)cc2cc1NC(=O)c1cccc(C(F)(F)F)c1] | N-(2-((1r,4r)-4-((3-(2-(2,4- dioxotetrahydropyrimidin-1(2H)-yl)-1,3- dioxoisoindolin-5-yl)-3,8- diazabicyclo[3.2.1]octan-8- yl)methyl)cyclohexyl)-6-methoxy-2H- indazol-5-yl)-3-(trifluoromethyl)benzamide |
| GT- 0866 8 | 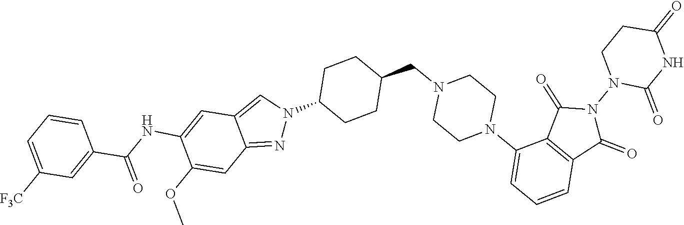 [SMILES: COc1cc2nn([C@H]3CC[C@H](CN4CCN(c5cccc6c5C(=O)N(N5CCC(=O)NC5=O)C6=O)CC4)CC3)cc2cc1NC(=O)c1cccc(C(F)(F)F)c1] | N-(2-((1r,4r)-4-((4-(2-(2,4- dioxotetrahydropyrimidin-1(2H)-yl)-1,3- dioxoisoindolin-4-yl)piperazin-1- yl)methyl)cyclohexyl)-6-methoxy-2H- indazol-5-yl)-3-(trifluoromethyl)benzamide |
| GT- 0866 9 | 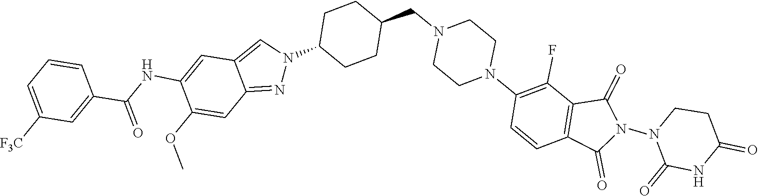 [SMILES: COc1cc2nn([C@H]3CC[C@H](CN4CCN(c5ccc6c(c5F)C(=O)N(N5CCC(=O)NC5=O)C6=O)CC4)CC3)cc2cc1NC(=O)c1cccc(C(F)(F)F)c1] | N-(2-((1r,4r)-4-((4-(2-(2,4- dioxotetrahydropyrimidin-1(2H)-yl)-4-fluoro- 1,3-dioxoisoindolin-5-yl)piperazin-1- yl)methyl)cyclohexyl)-6-methoxy-2H- indazol-5-yl)-3-(trifluoromethyl)benzamide |
| GT- 0867 0 | 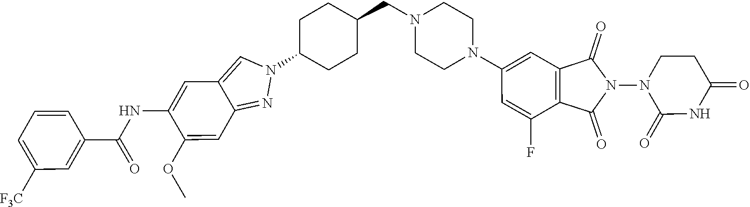 [SMILES: COc1cc2nn([C@H]3CC[C@H](CN4CCN(c5cc(F)c6c(c5)C(=O)N(N5CCC(=O)NC5=O)C6=O)CC4)CC3)cc2cc1NC(=O)c1cccc(C(F)(F)F)c1] | N-(2-((1r,4r)-4-((4-(2-(2,4- dioxotetrahydropyrimidin-1(2H)-yl)-7-fluoro- 1,3-dioxoisoindolin-5-yl)piperazin-1- yl)methyl)cyclohexyl)-6-methoxy-2H- indazol-5-yl)-3-(trifluoromethyl)benzamide |
| GT- 0867 1 | 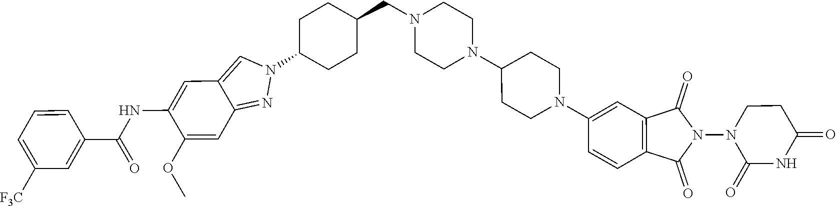 [SMILES: COc1cc2nn([C@H]3CC[C@H](CN4CCN(C5CCN(c6ccc7c(c6)C(=O)N(N6CCC(=O)NC6=O)C7=O)CC5)CC4)CC3)cc2cc1NC(=O)c1cccc(C(F)(F)F)c1] | N-(2-((1r,4r)-4-((4-(1-(2-(2,4- dioxotetrahydropyrimidin-1(2H)-yl)-1,3- dioxoisoindolin-5-yl)piperidin-4-yl)piperazin- 1-yl)methyl)cyclohexyl)-6-methoxy-2H- indazol-5-yl)-3-(trifluoromethyl)benzamide |
| GT- 0867 2 | 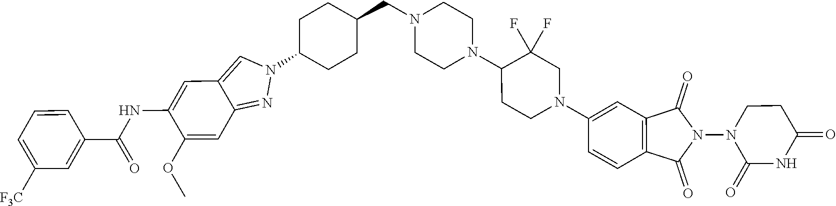 [SMILES: COc1cc2nn([C@H]3CC[C@H](CN4CCN(C5CCN(c6ccc7c(c6)C(=O)N(N6CCC(=O)NC6=O)C7=O)CC5(F)F)CC4)CC3)cc2cc1NC(=O)c1cccc(C(F)(F)F)c1] | N-(2-((1r,4r)-4-((4-(1-(2-(2,4- dioxotetrahydropyrimidin-1(2H)-yl)-1,3- dioxoisoindolin-5-yl)-3,3-difluoropiperidin-4- yl)piperazin-1-yl)methyl)cyclohexyl)-6- methoxy-2H-indazol-5-yl)-3- (trifluoromethyl)benzamide |
| GT- 0867 3 | 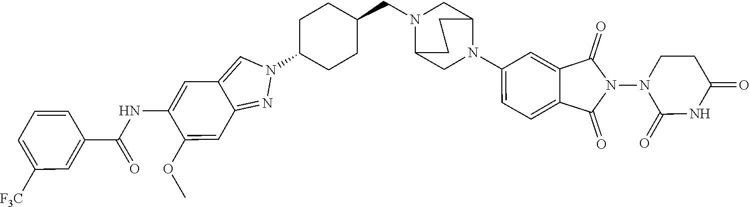 [SMILES: COc1cc2nn([C@H]3CC[C@H](CN4CC5CCC4CN5c4ccc5c(c4)C(=O)N(N4CCC(=O)NC4=O)C5=O)CC3)cc2cc1NC(=O)c1cccc(C(F)(F)F)c1] | N-(2-((1r,4r)-4-((5-(2-(2,4- dioxotetrahydropyrimidin-1(2H)-yl)-1,3- dioxoisoindolin-5-yl)-2,5- diazabicyclo[2.2.2]octan-2- yl)methyl)cyclohexyl)-6-methoxy-2H- indazol-5-yl)-3-(trifluoromethyl)benzamide |
| GT- 0582 2 | 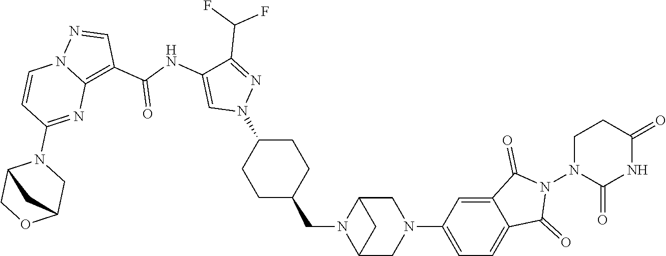 [SMILES: O=C1CCN(N2C(=O)c3ccc(N4CC5CC(C4)N5C[C@H]4CC[C@H](n5cc(NC(=O)c6cnn7ccc(N8C[C@H]9C[C@@H]8CO9)nc67)c(C(F)F)n5)CC4)cc3C2=O)C(=O)N1] | 5-((1R,4R)-2-oxa-5-azabicyclo[2.2.1]heptan- 5-yl)-N-(3-(difluoromethyl)-1-((1r,4R)-4-((3- (2-(2,4-dioxotetrahydropyrimidin-1(2H)-yl)- 1,3-dioxoisoindolin-5-yl)-3,6- diazabicyclo[3.1.1]heptan-6- yl)methyl)cyclohexyl)-1H-pyrazol-4- yl)pyrazolo[1,5-a]pyrimidine-3-carboxamide |
| GT- 0582 5 | 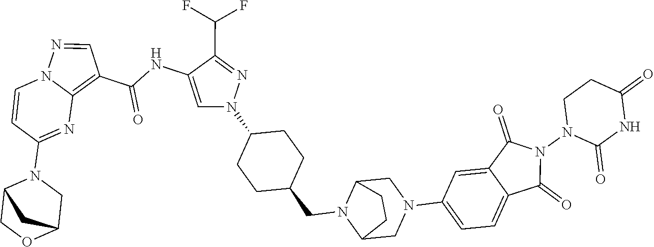 [SMILES: O=C1CCN(N2C(=O)c3ccc(N4CC5CCC(C4)N5C[C@H]4CC[C@H](n5cc(NC(=O)c6cnn7ccc(N8C[C@H]9C[C@@H]8CO9)nc67)c(C(F)F)n5)CC4)cc3C2=O)C(=O)N1] | 5-((1R,4R)-2-oxa-5-azabicyclo[2.2.1]heptan- 5-yl)-N-(3-(difluoromethyl)-1-((1r,4R)-4-((3- (2-(2,4-dioxotetrahydropyrimidin-1(2H)-yl)- 1,3-dioxoisoindolin-5-yl)-3,8- diazabicyclo[3.2.1]octan-8- yl)methyl)cyclohexyl)-1H-pyrazol-4- yl)pyrazolo[1,5-a]pyrimidine-3-carboxamide |
| GT- 0582 7 | 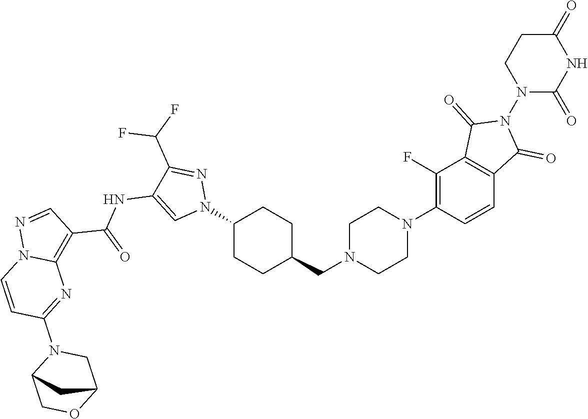 [SMILES: O=C1CCN(N2C(=O)c3ccc(N4CCN(C[C@H]5CC[C@H](n6cc(NC(=O)c7cnn8ccc(N9C[C@H]%10C[C@@H]9CO%10)nc78)c(C(F)F)n6)CC5)CC4)c(F)c3C2=O)C(=O)N1] | 5-((1R,4R)-2-oxa-5-azabicyclo[2.2.1]heptan- 5-yl)-N-(3-(difluoromethyl)-1-((1r,4R)-4-((4- (2-(2,4-dioxotetrahydropyrimidin-1(2H)-yl)- 4-fluoro-1,3-dioxoisoindolin-5-yl)piperazin- 1-yl)methyl)cyclohexyl)-1H-pyrazol-4- yl)pyrazolo[1,5-a]pyrimidine-3-carboxamide |
| GT- 0583 3 | 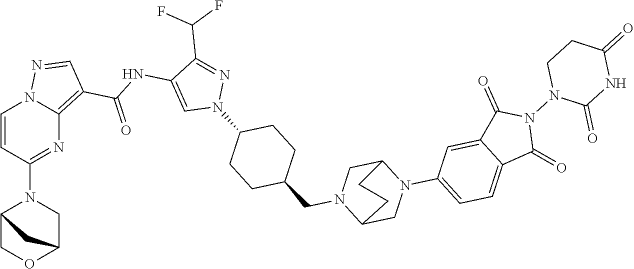 [SMILES: O=C1CCN(N2C(=O)c3ccc(N4CC5CCC4CN5C[C@H]4CC[C@H](n5cc(NC(=O)c6cnn7ccc(N8C[C@H]9C[C@@H]8CO9)nc67)c(C(F)F)n5)CC4)cc3C2=O)C(=O)N1] | 5-((1R,4R)-2-oxa-5-azabicyclo[2.2.1]heptan- 5-yl)-N-(3-(difluoromethyl)-1-((1r,4R)-4-((5- (2-(2,4-dioxotetrahydropyrimidin-1(2H)-yl)- 1,3-dioxoisoindolin-5-yl)-2,5- diazabicyclo[2.2.2]octan-2- yl)methyl)cyclohexyl)-1H-pyrazol-4- yl)pyrazolo[1,5-a]pyrimidine-3-carboxamide |
| GT- 0851 6 | 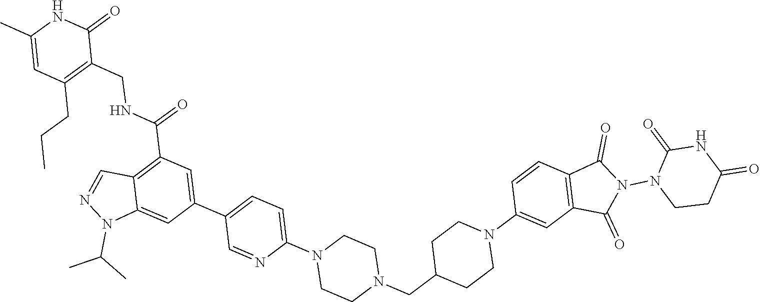 [SMILES: CCCc1cc(C)[nH]c(=O)c1CNC(=O)c1cc(-c2ccc(N3CCN(CC4CCN(c5ccc6c(c5)C(=O)N(N5CCC(=O)NC5=O)C6=O)CC4)CC3)nc2)cc2c1cnn2C(C)C] | 6-(6-(4-((1-(2-(2,4-dioxotetrahydropyrimidin- 1(2H)-yl)-1,3-dioxoisoindolin-5-yl)piperidin- 4-yl)methyl)piperazin-1-yl)pyridin-3-yl)-1- isopropyl-N-((6-methyl-2-oxo-4-propyl-1,2- dihydropyridin-3-yl)methyl)-1H-indazole-4- carboxamide |
| GT- 0851 7 | 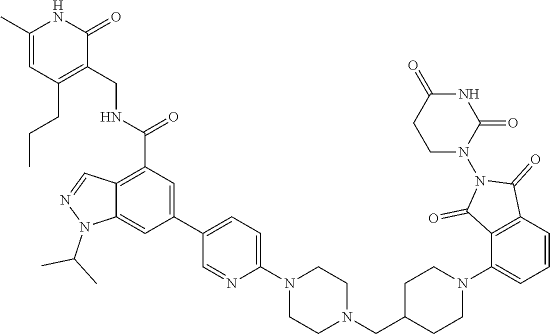 [SMILES: CCCc1cc(C)[nH]c(=O)c1CNC(=O)c1cc(-c2ccc(N3CCN(CC4CCN(c5cccc6c5C(=O)N(N5CCC(=O)NC5=O)C6=O)CC4)CC3)nc2)cc2c1cnn2C(C)C] | 6-(6-(4-((1-(2-(2,4-dioxotetrahydropyrimidin- 1(2H)-yl)-1,3-dioxoisoindolin-4-yl)piperidin- 4-yl)methyl)piperazin-1-yl)pyridin-3-yl)-1- isopropyl-N-((6-methyl-2-oxo-4-propyl-1,2- dihydropyridin-3-yl)methyl)-1H-indazole-4- carboxamide |
| GT- 0851 8 | 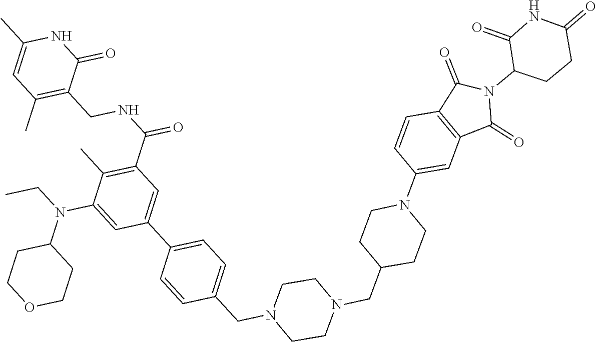 [SMILES: CCN(c1cc(-c2ccc(CN3CCN(CC4CCN(c5ccc6c(c5)C(=O)N(C5CCC(=O)NC5=O)C6=O)CC4)CC3)cc2)cc(C(=O)NCc2c(C)cc(C)[nH]c2=O)c1C)C1CCOCC1] | N-((4,6-dimethyl-2-oxo-1,2-dihydropyridin- 3-yl)methyl)-4′-((4-((1-(2-(2,4- dioxotetrahydropyrimidin-1(2H)-yl)-1,3- dioxoisoindolin-5-yl)piperidin-4- yl)methyl)piperazin-1-yl)methyl)-5- (ethyl(tetrahydro-2H-pyran-4-yl)amino)-4- methyl-[1,1′-biphenyl]-3-carboxamide |
| GT- 0851 9 | 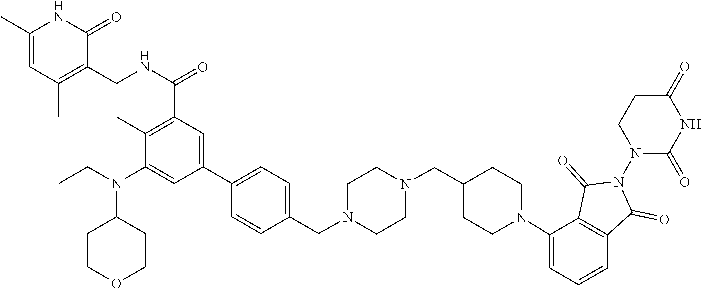 [SMILES: CCN(c1cc(-c2ccc(CN3CCN(CC4CCN(c5cccc6c5C(=O)N(N5CCC(=O)NC5=O)C6=O)CC4)CC3)cc2)cc(C(=O)NCc2c(C)cc(C)[nH]c2=O)c1C)C1CCOCC1] | N-((4,6-dimethyl-2-oxo-1,2-dihydropyridin- 3-yl)methyl)-4′-((4-((1-(2-(2,4- dioxotetrahydropyrimidin-1(2H)-yl)-1,3- dioxoisoindolin-4-yl)piperidin-4- yl)methyl)piperazin-1-yl)methyl)-5- (ethyl(tetrahydro-2H-pyran-4-yl)amino)-4- methyl-[1,1′-biphenyl]-3-carboxamide |
| GT- 0852 0 | 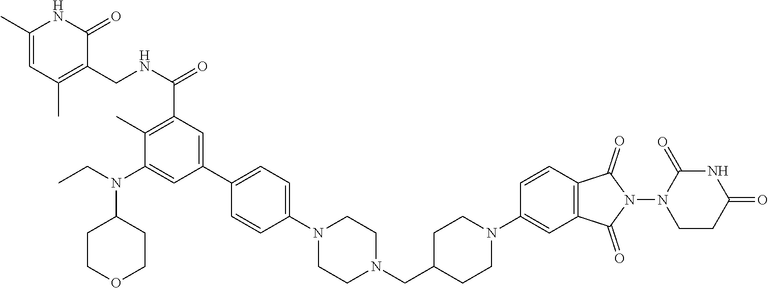 [SMILES: CCN(c1cc(-c2ccc(N3CCN(CC4CCN(c5ccc6c(c5)C(=O)N(N5CCC(=O)NC5=O)C6=O)CC4)CC3)cc2)cc(C(=O)NCc2c(C)cc(C)[nH]c2=O)c1C)C1CCOCC1] | N-((4,6-dimethyl-2-oxo-1,2-dihydropyridin- 3-yl)methyl)-4′-(4-((1-(2-(2,4- dioxotetrahydropyrimidin-1(2H)-yl)-1,3- dioxoisoindolin-5-yl)piperidin-4- yl)methyl)piperazin-1-yl)-5-(ethyl(tetrahydro- 2H-pyran-4-yl)amino)-4-methyl-[1,1′- biphenyl]-3-carboxamide |
| GT- 0852 1 | 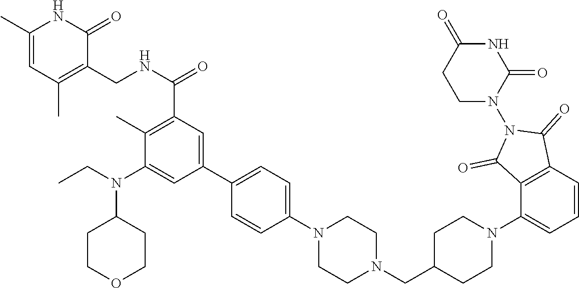 [SMILES: CCN(c1cc(-c2ccc(N3CCN(CC4CCN(c5cccc6c5C(=O)N(N5CCC(=O)NC5=O)C6=O)CC4)CC3)cc2)cc(C(=O)NCc2c(C)cc(C)[nH]c2=O)c1C)C1CCOCC1] | N-((4,6-dimethyl-2-oxo-1,2-dihydropyridin- 3-yl)methyl)-4′-(4-((1-(2-(2,4- dioxotetrahydropyrimidin-1(2H)-yl)-1,3- dioxoisoindolin-4-yl)piperidin-4- yl)methyl)piperazin-1-yl)-5-(ethyl(tetrahydro- 2H-pyran-4-yl)amino)-4-methyl-[1,1′- biphenyl]-3-carboxamide |
| GT- 0852 2 | 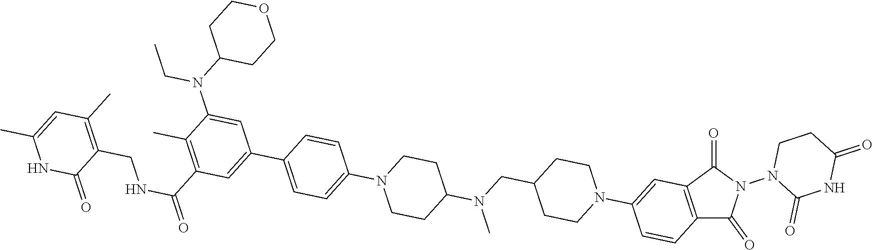 [SMILES: CCN(c1cc(-c2ccc(N3CCC(N(C)CC4CCN(c5ccc6c(c5)C(=O)N(N5CCC(=O)NC5=O)C6=O)CC4)CC3)cc2)cc(C(=O)NCc2c(C)cc(C)[nH]c2=O)c1C)C1CCOCC1] | N-((4,6-dimethyl-2-oxo-1,2-dihydropyridin- 3-yl)methyl)-4′-(4-(((1-(2-(2,4- dioxotetrahydropyrimidin-1(2H)-yl)-1,3- dioxoisoindolin-5-yl)piperidin-4- yl)methyl)(methyl)amino)piperidin-1-yl)-5- (ethyl(tetrahydro-2H-pyran-4-yl)amino)-4- methyl-[1,1′-biphenyl]-3-carboxamide |
| GT- 0852 3 | 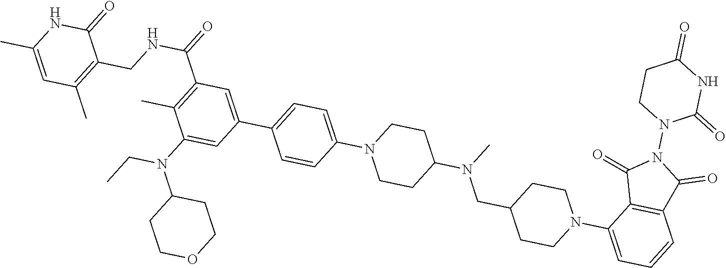 [SMILES: CCN(c1cc(-c2ccc(N3CCC(N(C)CC4CCN(c5cccc6c5C(=O)N(N5CCC(=O)NC5=O)C6=O)CC4)CC3)cc2)cc(C(=O)NCc2c(C)cc(C)[nH]c2=O)c1C)C1CCOCC1] | N-((4,6-dimethyl-2-oxo-1,2-dihydropyridin- 3-yl)methyl)-4′-(4-(((1-(2-(2,4- dioxotetrahydropyrimidin-1(2H)-yl)-1,3- dioxoisoindolin-4-yl)piperidin-4- yl)methyl)(methyl)amino)piperidin-1-yl)-5- (ethyl(tetrahydro-2H-pyran-4-yl)amino)-4- methyl-[1,1′-biphenyl]-3-carboxamide |
| GT- 0852 4 | 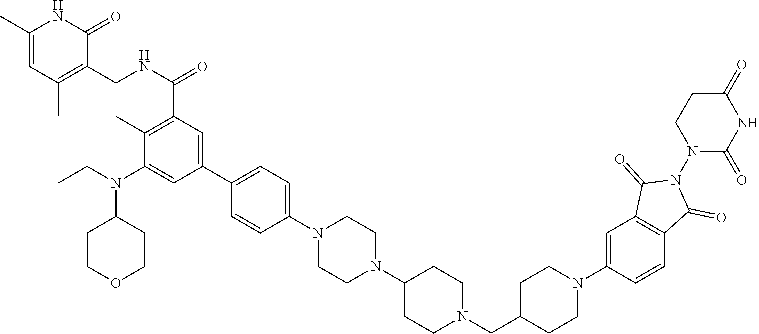 [SMILES: CCN(c1cc(-c2ccc(N3CCN(C4CCN(CC5CCN(c6ccc7c(c6)C(=O)N(N6CCC(=O)NC6=O)C7=O)CC5)CC4)CC3)cc2)cc(C(=O)NCc2c(C)cc(C)[nH]c2=O)c1C)C1CCOCC1] | N-((4,6-dimethyl-2-oxo-1,2-dihydropyridin- 3-yl)methyl)-4′-(4-(1-((1-(2-(2,4- dioxotetrahydropyrimidin-1(2H)-yl)-1,3- dioxoisoindolin-5-yl)piperidin-4- yl)methyl)piperidin-4-yl)piperazin-1-yl)-5- (ethyl(tetrahydro-2H-pyran-4-yl)amino)-4- methyl-[1,1′-biphenyl]-3-carboxamide |
| GT- 0852 5 | 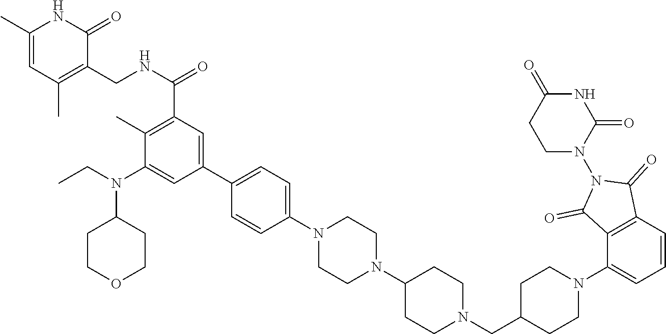 [SMILES: CCN(c1cc(-c2ccc(N3CCN(C4CCN(CC5CCN(c6cccc7c6C(=O)N(N6CCC(=O)NC6=O)C7=O)CC5)CC4)CC3)cc2)cc(C(=O)NCc2c(C)cc(C)[nH]c2=O)c1C)C1CCOCC1] | N-((4,6-dimethyl-2-oxo-1,2-dihydropyridin- 3-yl)methyl)-4′-(4-(1-((1-(2-(2,4- dioxotetrahydropyrimidin-1(2H)-yl)-1,3- dioxoisoindolin-4-yl)piperidin-4- yl)methyl)piperidin-4-yl)piperazin-1-yl)-5- (ethyl(tetrahydro-2H-pyran-4-yl)amino)-4- methyl-[1,1′-biphenyl]-3-carboxamide |
| GT- 0825 5 | 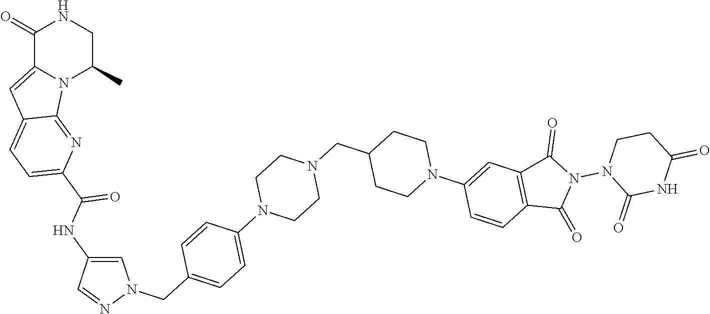 [SMILES: C[C@@H]1CNC(=O)c2cc3ccc(C(=O)Nc4cnn(Cc5ccc(N6CCN(CC7CCN(c8ccc9c(c8)C(=O)N(N8CCC(=O)NC8=O)C9=O)CC7)CC6)cc5)c4)nc3n21] | (R)-N-(1-(4-(4-((1-(2-(2,4- dioxotetrahydropyrimidin-1(2H)-yl)-1,3- dioxoisoindolin-5-yl)piperidin-4- yl)methyl)piperazin-1-yl)benzyl)-1H-pyrazol- 4-yl)-9-methyl-6-oxo-6,7,8,9- tetrahydropyrido [3′,2′:4,5]pyrrolo[1,2- a]pyrazine-2-carboxamide |
| GT- 0825 6 | 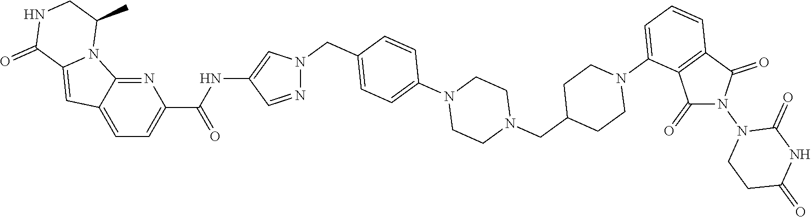 [SMILES: C[C@@H]1CNC(=O)c2cc3ccc(C(=O)Nc4cnn(Cc5ccc(N6CCN(CC7CCN(c8cccc9c8C(=O)N(N8CCC(=O)NC8=O)C9=O)CC7)CC6)cc5)c4)nc3n21] | (R)-N-(1-(4-(4-((1-(2-(2,4- dioxotetrahydropyrimidin-1(2H)-yl)-1,3- dioxoisoindolin-4-yl)piperidin-4- yl)methyl)piperazin-1-yl)benzyl)-1H-pyrazol- 4-yl)-9-methyl-6-oxo-6,7,8,9- tetrahydropyrido [3′,2′:4,5]pyrrolo[1,2- a]pyrazine-2-carboxamide |
| GT- 0834 3 | 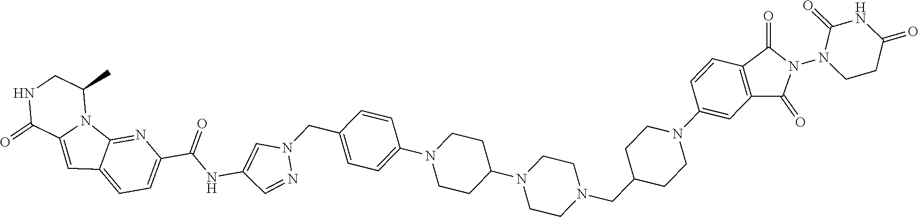 [SMILES: C[C@@H]1CNC(=O)c2cc3ccc(C(=O)Nc4cnn(Cc5ccc(N6CCC(N7CCN(CC8CCN(c9ccc%10c(c9)C(=O)N(N9CCC(=O)NC9=O)C%10=O)CC8)CC7)CC6)cc5)c4)nc3n21] | (R)-N-(1-(4-(4-(4-((1-(2-(2,4- dioxotetrahydropyrimidin-1(2H)-yl)-1,3- dioxoisoindolin-5-yl)piperidin-4- yl)methyl)piperazin-1-yl)piperidin-1- yl)benzyl)-1H-pyrazol-4-yl)-9-methyl-6-oxo- 6,7,8,9-tetrahydropyrido[3′,2′:4,5]pyrrolo[1,2- a]pyrazine-2-carboxamide |
| GT- 0834 4 | 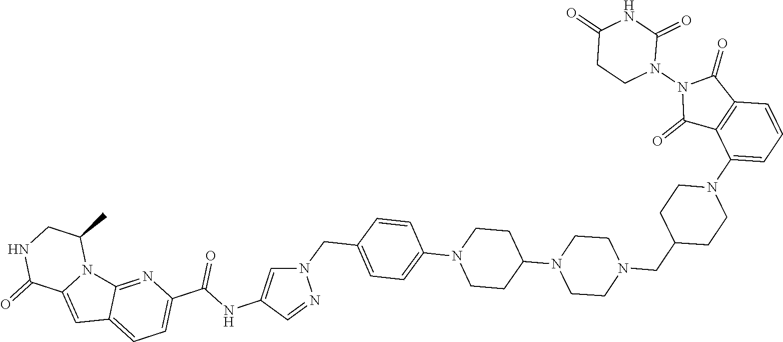 [SMILES: C[C@@H]1CNC(=O)c2cc3ccc(C(=O)Nc4cnn(Cc5ccc(N6CCC(N7CCN(CC8CCN(c9cccc%10c9C(=O)N(N9CCC(=O)NC9=O)C%10=O)CC8)CC7)CC6)cc5)c4)nc3n21] | (R)-N-(1-(4-(4-(4-((1-(2-(2,4- dioxotetrahydropyrimidin-1(2H)-yl)-1,3- dioxoisoindolin-4-yl)piperidin-4- yl)methyl)piperazin-1-yl)piperidin-1- yl)benzyl)-1H-pyrazol-4-yl)-9-methyl-6-oxo- 6,7,8,9-tetrahydropyrido[3′,2′:4,5]pyrrolo[1,2- a]pyrazine-2-carboxamide |
| GT- 0682 8 | 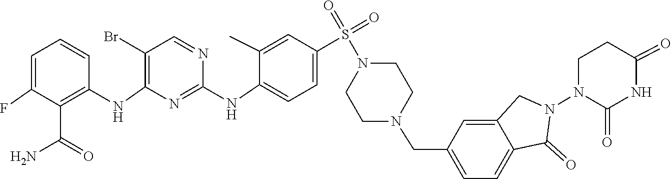 [SMILES: Cc1cc(S(=O)(=O)N2CCN(Cc3ccc4c(c3)CN(N3CCC(=O)NC3=O)C4=O)CC2)ccc1Nc1ncc(Br)c(Nc2cccc(F)c2C(N)=O)n1] | 2-((5-bromo-2-((4-((4-((2-(2,4- dioxotetrahydropyrimidin-1(2H)-yl)-1- oxoisoindolin-5-yl)methyl)piperazin-1- yl)sulfonyl)-2- methylphenyl)amino)pyrimidin-4-yl)amino)- 6-fluorobenzamide |
| GT- 0682 9 | 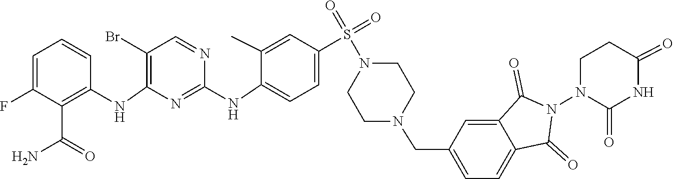 [SMILES: Cc1cc(S(=O)(=O)N2CCN(Cc3ccc4c(c3)C(=O)N(N3CCC(=O)NC3=O)C4=O)CC2)ccc1Nc1ncc(Br)c(Nc2cccc(F)c2C(N)=O)n1] | 2-((5-bromo-2-((4-((4-((2-(2,4- dioxotetrahydropyrimidin-1(2H)-yl)-1,3- dioxoisoindolin-5-yl)methyl)piperazin-1- yl)sulfonyl)-2- methylphenyl)amino)pyrimidin-4-yl)amino)- 6-fluorobenzamide |
| GT- 0833 7 | 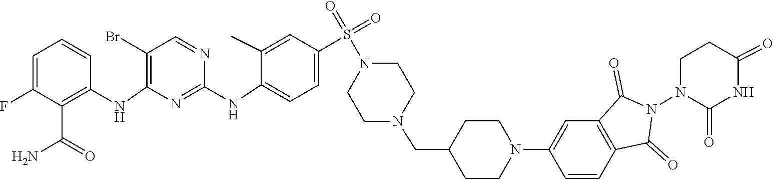 [SMILES: Cc1cc(S(=O)(=O)N2CCN(CC3CCN(c4ccc5c(c4)C(=O)N(N4CCC(=O)NC4=O)C5=O)CC3)CC2)ccc1Nc1ncc(Br)c(Nc2cccc(F)c2C(N)=O)n1] | 2-((5-bromo-2-((4-((4-((1-(2-(2,4- dioxotetrahydropyrimidin-1(2H)-yl)-1,3- dioxoisoindolin-5-yl)piperidin-4- yl)methyl)piperazin-1-yl)sulfonyl)-2- methylphenyl)amino)pyrimidin-4-yl)amino)- 6-fluorobenzamide |
| GT- 0833 8 | 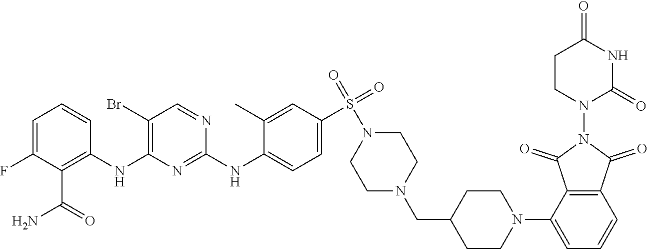 [SMILES: Cc1cc(S(=O)(=O)N2CCN(CC3CCN(c4cccc5c4C(=O)N(N4CCC(=O)NC4=O)C5=O)CC3)CC2)ccc1Nc1ncc(Br)c(Nc2cccc(F)c2C(N)=O)n1] | 2-((5-bromo-2-((4-((4-((1-(2-(2,4- dioxotetrahydropyrimidin-1(2H)-yl)-1,3- dioxoisoindolin-4-yl)piperidin-4- yl)methyl)piperazin-1-yl)sulfonyl)-2- methylphenyl)amino)pyrimidin-4-yl)amino)- 6-fluorobenzamide |
| GT- 0674 8 | 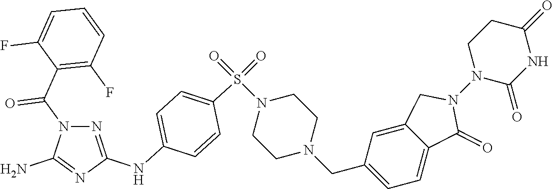 [SMILES: Nc1nc(Nc2ccc(S(=O)(=O)N3CCN(Cc4ccc5c(c4)CN(N4CCC(=O)NC4=O)C5=O)CC3)cc2)nn1C(=O)c1c(F)cccc1F] | 1-(5-((4-((4-((5-amino-1-(2,6- difluorobenzoyl)-1H-1,2,4-triazol-3- yl)amino)phenyl)sulfonyl)piperazin-1- yl)methyl)-1-oxoisoindolin-2- yl)dihydropyrimidine-2,4(1H,3H)-dione |
| GT- 0674 9 | 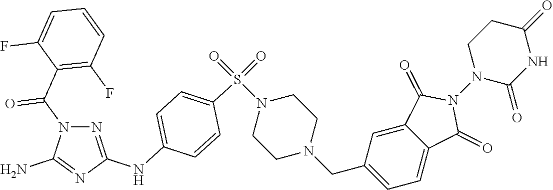 [SMILES: Nc1nc(Nc2ccc(S(=O)(=O)N3CCN(Cc4ccc5c(c4)C(=O)N(N4CCC(=O)NC4=O)C5=O)CC3)cc2)nn1C(=O)c1c(F)cccc1F] | 5-((4-((4-((5-amino-1-(2,6-difluorobenzoyl)- 1H-1,2,4-triazol-3- yl)amino)phenyl)sulfonyl)piperazin-1- yl)methyl)-2-(2,4-dioxotetrahydropyrimidin- 1(2H)-yl)isoindoline-1,3-dione |
| GT- 0833 9 | 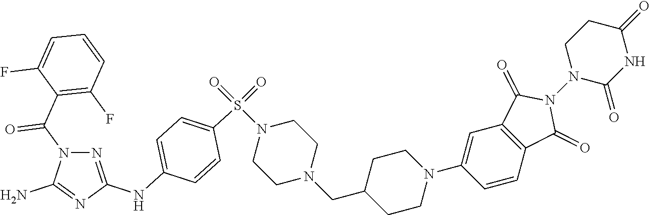 [SMILES: Nc1nc(Nc2ccc(S(=O)(=O)N3CCN(CC4CCN(c5ccc6c(c5)C(=O)N(N5CCC(=O)NC5=O)C6=O)CC4)CC3)cc2)nn1C(=O)c1c(F)cccc1F] | 5-(4-((4-((4-((5-amino-1-(2,6- difluorobenzoyl)-1H-1,2,4-triazol-3- yl)amino)phenyl)sulfonyl)piperazin-1- yl)methyl)piperidin-1-yl)-2-(2,4- dioxotetrahydropyrimidin-1(2H)- yl)isoindoline-1,3-dione |
| GT- 0834 0 | 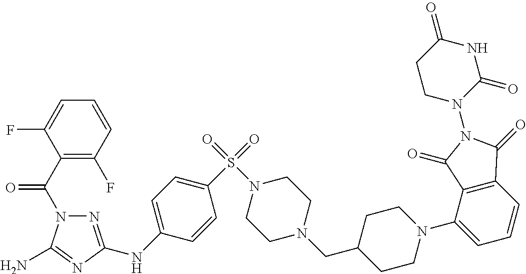 | 4-(4-((4-((4-((5-amino-1-(2,6- difluorobenzoyl)-1H-1,2,4-triazol-3- yl)amino)phenyl)sulfonyl)piperazin-1- yl)methyl)piperidin-1-yl)-2-(2,4- dioxotetrahydropyrimidin-1(2H)- yl)isoindoline-1,3-dione |
| GT- 0685 2 | 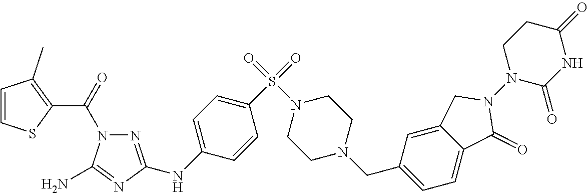 | 1-(5-((4-((4-((5-amino-1-(3-methylthiophene- 2-carbonyl)-1H-1,2,4-triazol-3- yl)amino)phenyl)sulfonyl)piperazin-1- yl)methyl)-1-oxoisoindolin-2- yl)dihydropyrimidine-2,4(1H,3H)-dione |
| GT- 0685 3 | 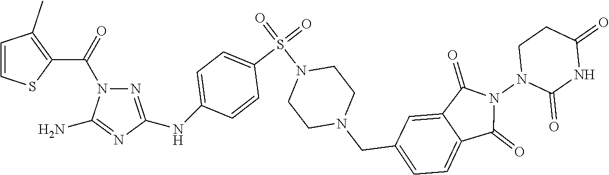 [SMILES: Cc1ccsc1C(=O)n1nc(Nc2ccc(S(=O)(=O)N3CCN(Cc4ccc5c(c4)C(=O)N(N4CCC(=O)NC4=O)C5=O)CC3)cc2)nc1N] | 5-((4-((4-((5-amino-1-(3-methylthiophene-2- carbonyl)-1H-1,2,4-triazol-3- yl)amino)phenyl)sulfonyl)piperazin-1- yl)methyl)-2-(2,4-dioxotetrahydropyrimidin- 1(2H)-yl)isoindoline-1,3-dione |
| GT- 0834 1 | 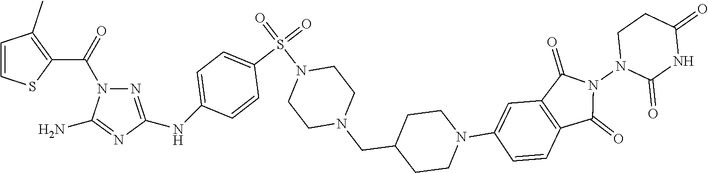 [SMILES: Cc1ccsc1C(=O)n1nc(Nc2ccc(S(=O)(=O)N3CCN(CC4CCN(c5ccc6c(c5)C(=O)N(N5CCC(=O)NC5=O)C6=O)CC4)CC3)cc2)nc1N] | 5-(4-((4-((4-((5-amino-1-(3-methylthiophene- 2-carbonyl)-1H-1,2,4-triazol-3- yl)amino)phenyl)sulfonyl)piperazin-1- yl)methyl)piperidin-1-yl)-2-(2,4- dioxotetrahydropyrimidin-1(2H)- yl)isoindoline-1,3-dione |
| GT- 0834 2 | 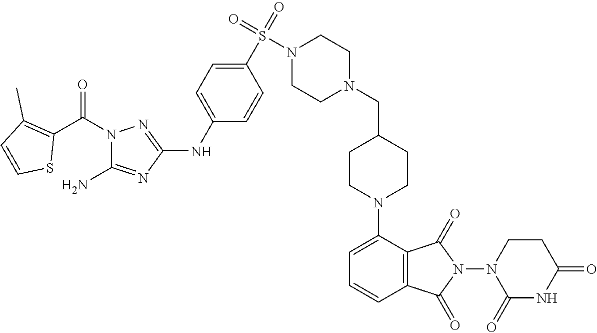 [SMILES: Cc1ccsc1C(=O)n1nc(Nc2ccc(S(=O)(=O)N3CCN(CC4CCN(c5cccc6c5C(=O)N(N5CCC(=O)NC5=O)C6=O)CC4)CC3)cc2)nc1N] | 4-(4-((4-((4-((5-amino-1-(3-methylthiophene- 2-carbonyl)-1H-1,2,4-triazol-3- yl)amino)phenyl)sulfonyl)piperazin-1- yl)methyl)piperidin-1-yl)-2-(2,4- dioxotetrahydropyrimidin-1(2H)- yl)isoindoline-1,3-dione |
| GT- 0839 4 | 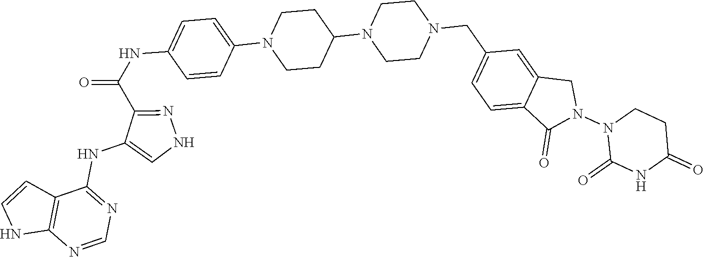 [SMILES: O=C1CCN(N2Cc3cc(CN4CCN(C5CCN(c6ccc(NC(=O)c7n[nH]cc7Nc7ncnc8[nH]ccc78)cc6)CC5)CC4)ccc3C2=O)C(=O)N1] | 4-((7H-pyrrolo[2,3-d]pyrimidin-4-yl)amino)- N-(4-(4-(4-((2-(2,4- dioxotetrahydropyrimidin-1(2H)-yl)-1- oxoisoindolin-5-yl)methyl)piperazin-1- yl)piperidin-1-yl)phenyl)-1H-pyrazole-3- carboxamide |
| GT- 0839 5 | 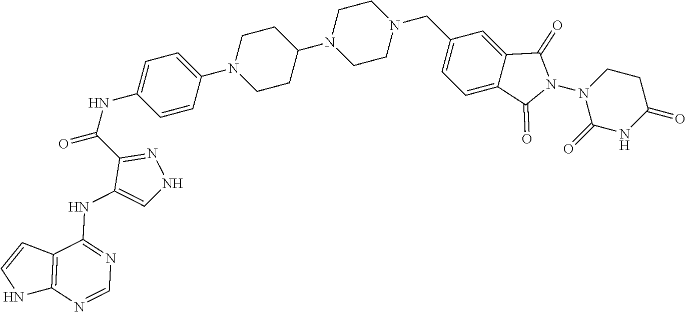 | 4-((7H-pyrrolo[2,3-d]pyrimidin-4-yl)amino)- N-(4-(4-(4-((2-(2,4- dioxotetrahydropyrimidin-1(2H)-yl)-1,3- dioxoisoindolin-5-yl)methyl)piperazin-1- yl)piperidin-1-yl)phenyl)-1H-pyrazole-3- carboxamide |
| GT- 0839 6 | 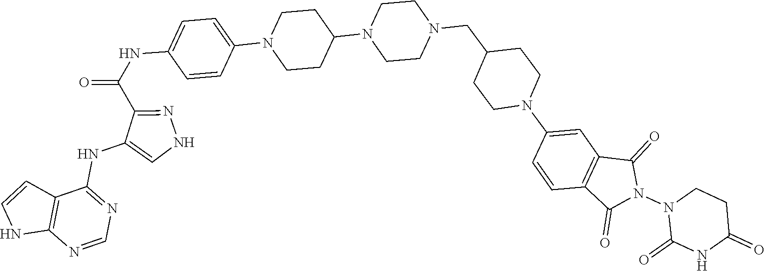 [SMILES: O=C1CCN(N2C(=O)c3ccc(N4CCC(CN5CCN(C6CCN(c7ccc(NC(=O)c8n[nH]cc8Nc8ncnc9[nH]ccc89)cc7)CC6)CC5)CC4)cc3C2=O)C(=O)N1] | 4-((7H-pyrrolo[2,3-d]pyrimidin-4-yl)amino)- N-(4-(4-(4-((1-(2-(2,4- dioxotetrahydropyrimidin-1(2H)-yl)-1,3- dioxoisoindolin-5-yl)piperidin-4- yl)methyl)piperazin-1-yl)piperidin-1- yl)phenyl)-1H-pyrazole-3-carboxamide |
| GT- 0839 7 | 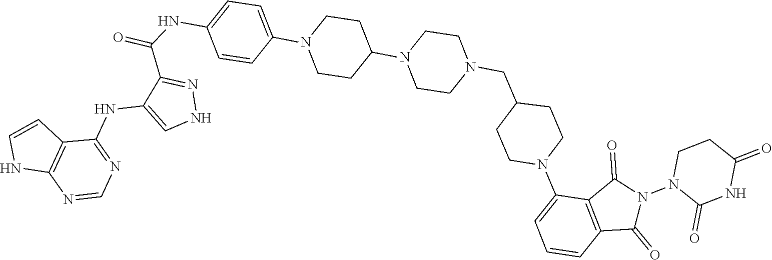 [SMILES: O=C1CCN(N2C(=O)c3cccc(N4CCC(CN5CCN(C6CCN(c7ccc(NC(=O)c8n[nH]cc8Nc8ncnc9[nH]ccc89)cc7)CC6)CC5)CC4)c3C2=O)C(=O)N1] | 4-((7H-pyrrolo[2,3-d]pyrimidin-4-yl)amino)- N-(4-(4-(4-((1-(2-(2,4- dioxotetrahydropyrimidin-1(2H)-yl)-1,3- dioxoisoindolin-4-yl)piperidin-4- yl)methyl)piperazin-1-yl)piperidin-1- yl)phenyl)-1H-pyrazole-3-carboxamide |
| GT- 0853 5 | 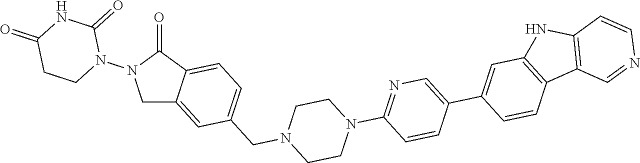 [SMILES: O=C1CCN(N2Cc3cc(CN4CCN(c5ccc(-c6ccc7c(c6)[nH]c6ccncc67)cn5)CC4)ccc3C2=O)C(=O)N1] | 1-(5-((4-(5-(5H-pyrido[4,3-b]indol-7- yl)pyridin-2-yl)piperazin-1-yl)methyl)-1- oxoisoindolin-2-yl)dihydropyrimidine- 2,4(1H,3H)-dione |
| GT- 0854 2 | 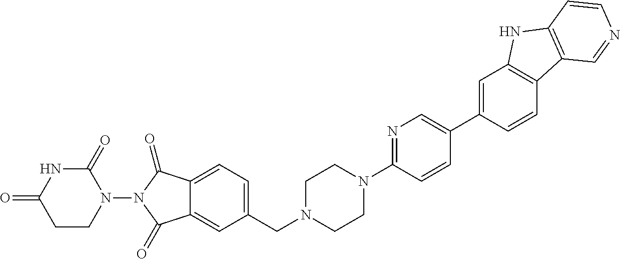 [SMILES: O=C1CCN(N2C(=O)c3ccc(CN4CCN(c5ccc(-c6ccc7c(c6)[nH]c6ccncc67)cn5)CC4)cc3C2=O)C(=O)N1] | 5-((4-(5-(5H-pyrido[4,3-b]indol-7-yl)pyridin- 2-yl)piperazin-1-yl)methyl)-2-(2,4- dioxotetrahydropyrimidin-1(2H)- yl)isoindoline-1,3-dione |
| GT- 0855 7 | 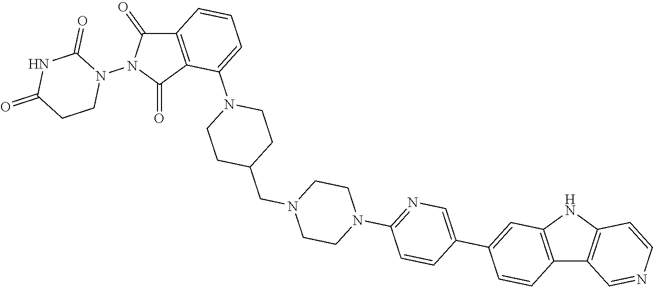 | 4-(4-((4-(5-(5H-pyrido[4,3-b]indol-7- yl)pyridin-2-yl)piperazin-1- yl)methyl)piperidin-1-yl)-2-(2,4- dioxotetrahydropyrimidin-1(2H)- yl)isoindoline-1,3-dione |
| GT- 0853 6 | 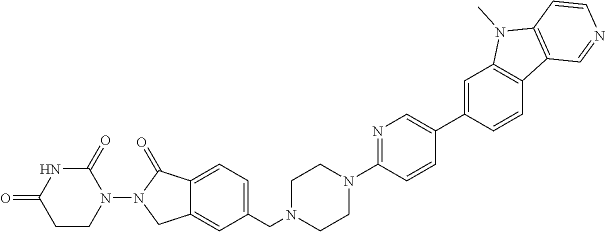 [SMILES: Cn1c2ccncc2c2ccc(-c3ccc(N4CCN(Cc5ccc6c(c5)CN(N5CCC(=O)NC5=O)C6=O)CC4)nc3)cc21] | 1-(5-((4-(5-(5-methyl-5H-pyrido[4,3-b]indol- 7-yl)pyridin-2-yl)piperazin-1-yl)methyl)-1- oxoisoindolin-2-yl)dihydropyrimidine- 2,4(1H,3H)-dione |
| GT- 0854 3 | 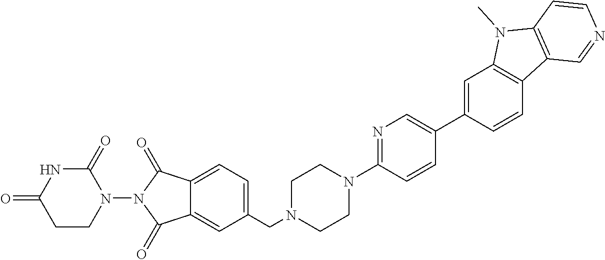 [SMILES: Cn1c2ccncc2c2ccc(-c3ccc(N4CCN(Cc5ccc6c(c5)C(=O)N(N5CCC(=O)NC5=O)C6=O)CC4)nc3)cc21] | 2-(2,4-dioxotetrahydropyrimidin-1(2H)-yl)-5- ((4-(5-(5-methyl-5H-pyrido[4,3-b]indol-7- yl)pyridin-2-yl)piperazin-1- yl)methyl)isoindoline-1,3-dione |
| GT- 0855 0 | 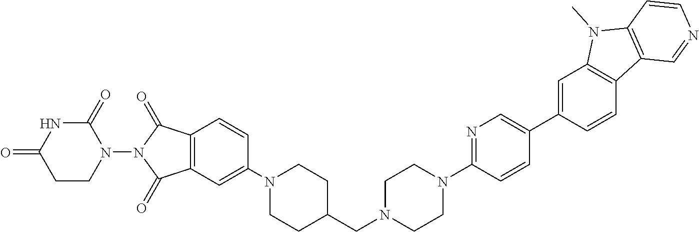 [SMILES: Cn1c2ccncc2c2ccc(-c3ccc(N4CCN(CC5CCN(c6ccc7c(c6)C(=O)N(N6CCC(=O)NC6=O)C7=O)CC5)CC4)nc3)cc21] | 2-(2,4-dioxotetrahydropyrimidin-1(2H)-yl)-5- (4-((4-(5-(5-methyl-5H-pyrido[4,3-b]indol-7- yl)pyridin-2-yl)piperazin-1- yl)methyl)piperidin-1-yl)isoindoline-1,3- dione |
| GT- 0855 8 | 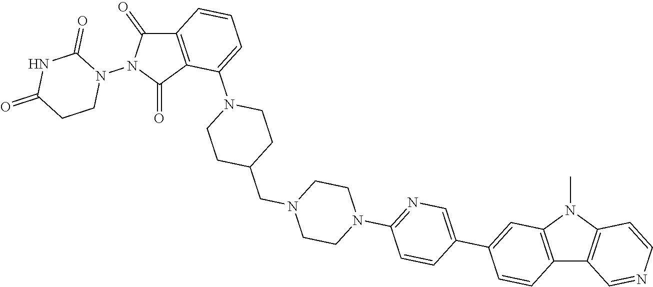 [SMILES: Cn1c2ccncc2c2ccc(-c3ccc(N4CCN(CC5CCN(c6cccc7c6C(=O)N(N6CCC(=O)NC6=O)C7=O)CC5)CC4)nc3)cc21] | 2-(2,4-dioxotetrahydropyrimidin-1(2H)-yl)-4- (4-((4-(5-(5-methyl-5H-pyrido[4,3-b]indol-7- yl)pyridin-2-yl)piperazin-1- yl)methyl)piperidin-1-yl)isoindoline-1,3- dione |
| GT- 0853 7 | 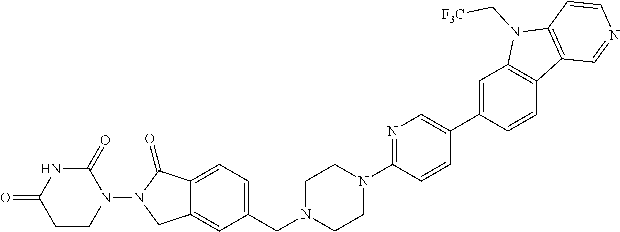 [SMILES: O=C1CCN(N2Cc3cc(CN4CCN(c5ccc(-c6ccc7c8cnccc8n(CC(F)(F)F)c7c6)cn5)CC4)ccc3C2=O)C(=O)N1] | 1-(1-oxo-5-((4-(5-(5-(2,2,2-trifluoroethyl)- 5H-pyrido[4,3-b]indol-7-yl)pyridin-2- yl)piperazin-1-yl)methyl)isoindolin-2- yl)dihydropyrimidine-2,4(1H,3H)-dione |
| GT- 0854 4 | 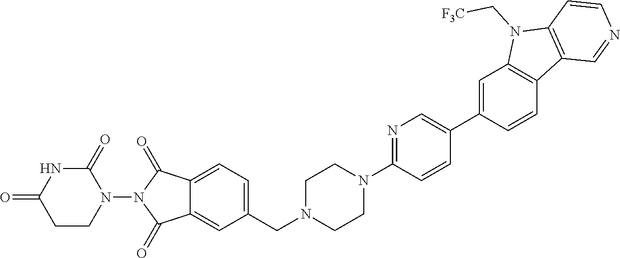 [SMILES: O=C1CCN(N2C(=O)c3ccc(CN4CCN(c5ccc(-c6ccc7c8cnccc8n(CC(F)(F)F)c7c6)cn5)CC4)cc3C2=O)C(=O)N1] | 2-(2,4-dioxotetrahydropyrimidin-1(2H)-yl)-5- ((4-(5-(5-(2,2,2-trifluoroethyl)-5H- pyrido[4,3-b]indol-7-yl)pyridin-2- yl)piperazin-1-yl)methyl)isoindoline-1,3- dione |
| GT- 0855 1 | 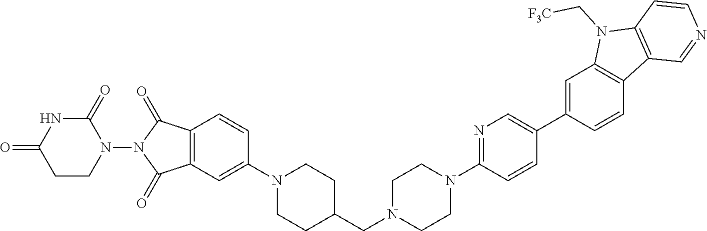 [SMILES: O=C1CCN(N2C(=O)c3ccc(N4CCC(CN5CCN(c6ccc(-c7ccc8c9cnccc9n(CC(F)(F)F)c8c7)cn6)CC5)CC4)cc3C2=O)C(=O)N1] | 2-(2,4-dioxotetrahydropyrimidin-1(2H)-yl)-5- (4-((4-(5-(5-(2,2,2-trifluoroethyl)-5H- pyrido[4,3-b]indol-7-yl)pyridin-2- yl)piperazin-1-yl)methyl)piperidin-1- yl)isoindoline-1,3-dione |
| GT- 0855 9 | 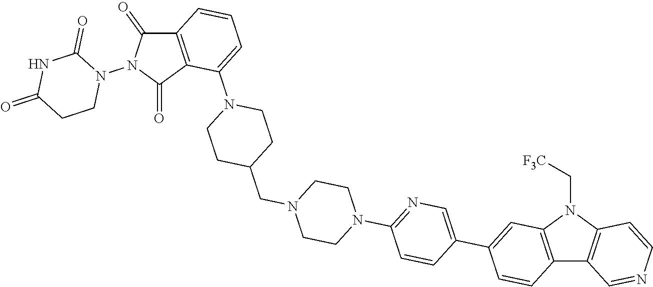 [SMILES: O=C1CCN(N2C(=O)c3cccc(N4CCC(CN5CCN(c6ccc(-c7ccc8c9cnccc9n(CC(F)(F)F)c8c7)cn6)CC5)CC4)c3C2=O)C(=O)N1] | 2-(2,4-dioxotetrahydropyrimidin-1(2H)-yl)-4- (4-((4-(5-(5-(2,2,2-trifluoroethyl)-5H- pyrido[4,3-b]indol-7-yl)pyridin-2- yl)piperazin-1-yl)methyl)piperidin-1- yl)isoindoline-1,3-dione |
| GT- 0853 8 | 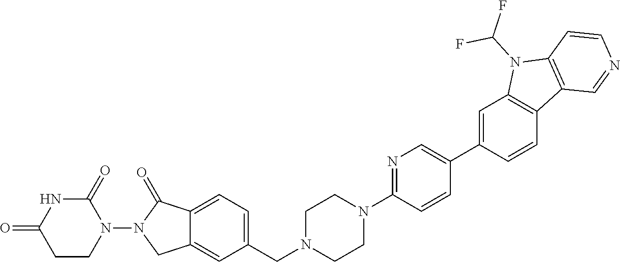 [SMILES: O=C1CCN(N2Cc3cc(CN4CCN(c5ccc(-c6ccc7c8cnccc8n(C(F)F)c7c6)cn5)CC4)ccc3C2=O)C(=O)N1] | 1-(5-((4-(5-(5-(difluoromethyl)-5H- pyrido[4,3-b]indol-7-yl)pyridin-2- yl)piperazin-1-yl)methyl)-1-oxoisoindolin-2- yl)dihydropyrimidine-2,4(1H,3H)-dione |
| GT- 0854 5 | 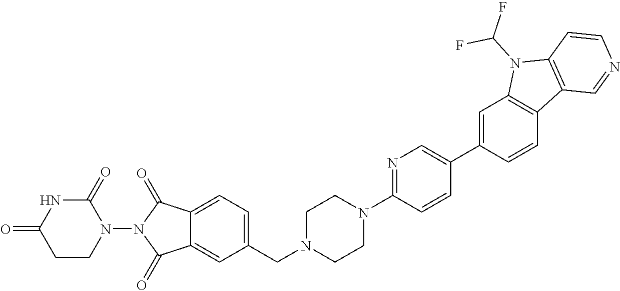 [SMILES: O=C1CCN(N2C(=O)c3ccc(CN4CCN(c5ccc(-c6ccc7c8cnccc8n(C(F)F)c7c6)cn5)CC4)cc3C2=O)C(=O)N1] | 5-((4-(5-(5-(difluoromethyl)-5H-pyrido[4,3- b]indol-7-yl)pyridin-2-yl)piperazin-1- yl)methyl)-2-(2,4-dioxotetrahydropyrimidin- 1(2H)-yl)isoindoline-1,3-dione |
| GT- 0855 2 | 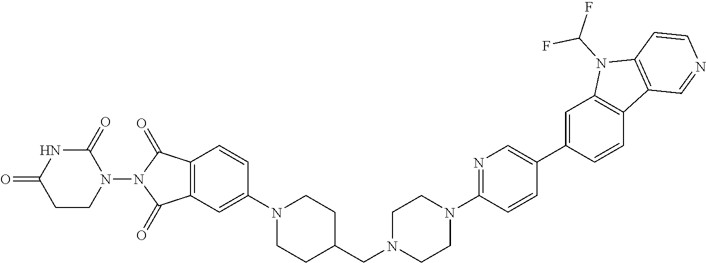 [SMILES: O=C1CCN(N2C(=O)c3ccc(N4CCC(CN5CCN(c6ccc(-c7ccc8c9cnccc9n(C(F)F)c8c7)cn6)CC5)CC4)cc3C2=O)C(=O)N1] | 5-(4-((4-(5-(5-(difluoromethyl)-5H- pyrido[4,3-b]indol-7-yl)pyridin-2- yl)piperazin-1-yl)methyl)piperidin-1-yl)-2- (2,4-dioxotetrahydropyrimidin-1(2H)- yl)isoindoline-1,3-dione |
| GT- 0856 0 | 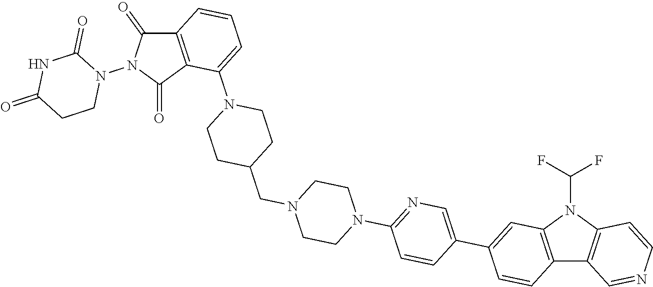 [SMILES: O=C1CCN(N2C(=O)c3cccc(N4CCC(CN5CCN(c6ccc(-c7ccc8c9cnccc9n(C(F)F)c8c7)cn6)CC5)CC4)c3C2=O)C(=O)N1] | 4-(4-((4-(5-(5-(difluoromethyl)-5H- pyrido[4,3-b]indol-7-yl)pyridin-2- yl)piperazin-1-yl)methyl)piperidin-1-yl)-2- (2,4-dioxotetrahydropyrimidin-1(2H)- yl)isoindoline-1,3-dione |
| GT- 0853 9 | 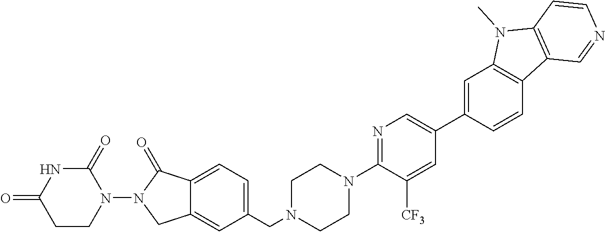 [SMILES: Cn1c2ccncc2c2ccc(-c3cnc(N4CCN(Cc5ccc6c(c5)CN(N5CCC(=O)NC5=O)C6=O)CC4)c(C(F)(F)F)c3)cc21] | 1-(5-((4-(5-(5-methyl-5H-pyrido[4,3-b]indol- 7-yl)-3-(trifluoromethyl)pyridin-2- yl)piperazin-1-yl)methyl)-1-oxoisoindolin-2- yl)dihydropyrimidine-2,4(1H,3H)-dione |
| GT- 0854 6 | 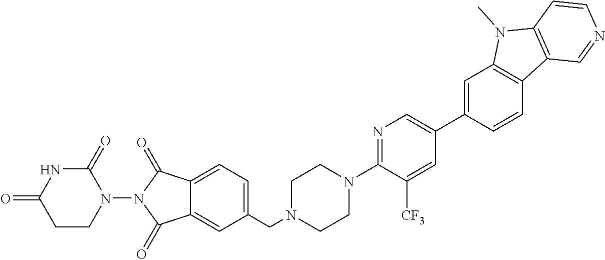 [SMILES: Cn1c2ccncc2c2ccc(-c3cnc(N4CCN(Cc5ccc6c(c5)C(=O)N(N5CCC(=O)NC5=O)C6=O)CC4)c(C(F)(F)F)c3)cc21] | 2-(2,4-dioxotetrahydropyrimidin-1(2H)-yl)-5- ((4-(5-(5-methyl-5H-pyrido[4,3-b]indol-7-yl)- 3-(trifluoromethyl)pyridin-2-yl)piperazin-1- yl)methyl)isoindoline-1,3-dione |
| GT- 0855 3 | 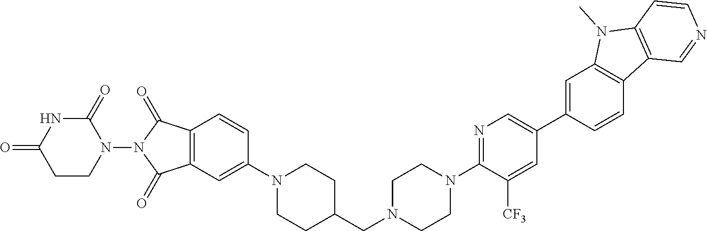 [SMILES: Cn1c2ccncc2c2ccc(-c3cnc(N4CCN(CC5CCN(c6ccc7c(c6)C(=O)N(N6CCC(=O)NC6=O)C7=O)CC5)CC4)c(C(F)(F)F)c3)cc21] | 2-(2,4-dioxotetrahydropyrimidin-1(2H)-yl)-5- (4-((4-(5-(5-methyl-5H-pyrido[4,3-b]indol-7- yl)-3-(trifluoromethyl)pyridin-2-yl)piperazin- 1-yl)methyl)piperidin-1-yl)isoindoline-1,3- dione |
| GT- 0856 1 | 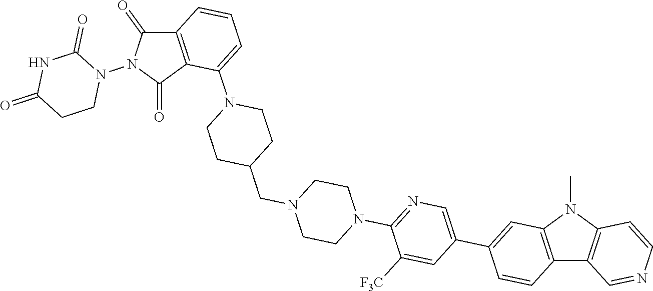 [SMILES: Cn1c2ccncc2c2ccc(-c3cnc(N4CCN(CC5CCN(c6cccc7c6C(=O)N(N6CCC(=O)NC6=O)C7=O)CC5)CC4)c(C(F)(F)F)c3)cc21] | 2-(2,4-dioxotetrahydropyrimidin-1(2H)-yl)-4- (4-((4-(5-(5-methyl-5H-pyrido[4,3-b]indol-7- yl)-3-(trifluoromethyl)pyridin-2-yl)piperazin- 1-yl)methyl)piperidin-1-yl)isoindoline-1,3- dione |
| GT- 0854 7 | 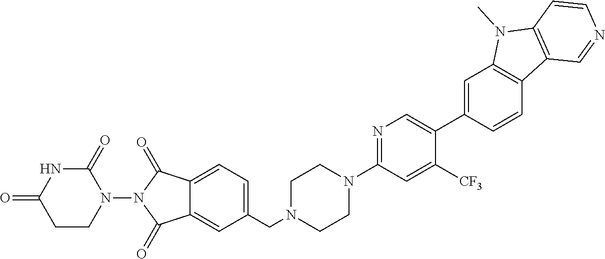 [SMILES: Cn1c2ccncc2c2ccc(-c3cnc(N4CCN(Cc5ccc6c(c5)C(=O)N(N5CCC(=O)NC5=O)C6=O)CC4)cc3C(F)(F)F)cc21] | 2-(2,4-dioxotetrahydropyrimidin-1(2H)-yl)-5- ((4-(5-(5-methyl-5H-pyrido[4,3-b]indol-7-yl)- 4-(trifluoromethyl)pyridin-2-yl)piperazin-1- yl)methyl)isoindoline-1,3-dione |
| GT- 0855 4 | 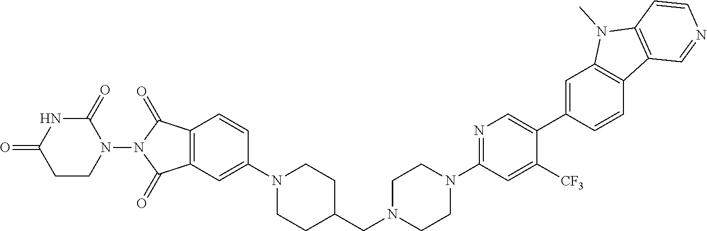 [SMILES: Cn1c2ccncc2c2ccc(-c3cnc(N4CCN(CC5CCN(c6ccc7c(c6)C(=O)N(N6CCC(=O)NC6=O)C7=O)CC5)CC4)cc3C(F)(F)F)cc21] | 2-(2,4-dioxotetrahydropyrimidin-1(2H)-yl)-5- (4-((4-(5-(5-methyl-5H-pyrido[4,3-b]indol-7- yl)-4-(trifluoromethyl)pyridin-2-yl)piperazin- 1-yl)methyl)piperidin-1-yl)isoindoline-1,3- dione |
| GT- 0856 2 | 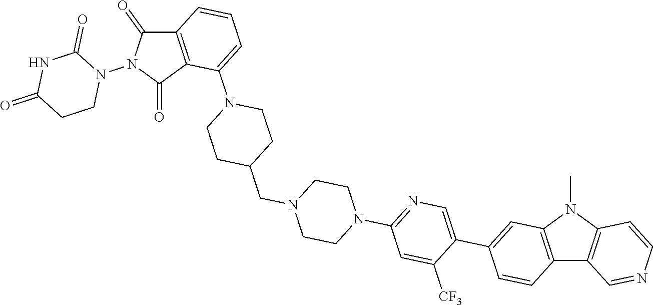 [SMILES: Cn1c2ccncc2c2ccc(-c3cnc(N4CCN(CC5CCN(c6cccc7c6C(=O)N(N6CCC(=O)NC6=O)C7=O)CC5)CC4)cc3C(F)(F)F)cc21] | 2-(2,4-dioxotetrahydropyrimidin-1(2H)-yl)-4- (4-((4-(5-(5-methyl-5H-pyrido[4,3-b]indol-7- yl)-4-(trifluoromethyl)pyridin-2-yl)piperazin- 1-yl)methyl)piperidin-1-yl)isoindoline-1,3- dione |
| GT- 0854 0 | 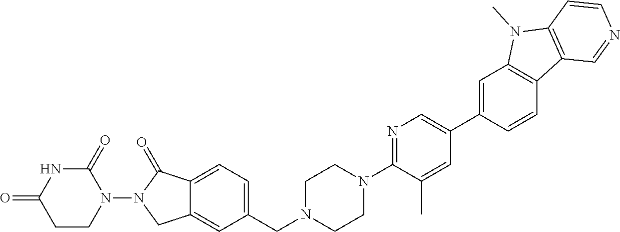 [SMILES: Cc1cc(-c2ccc3c4cnccc4n(C)c3c2)cnc1N1CCN(Cc2ccc3c(c2)CN(N2CCC(=O)NC2=O)C3=O)CC1] | 1-(5-((4-(3-methyl-5-(5-methyl-5H- pyrido[4,3-b]indol-7-yl)pyridin-2- yl)piperazin-1-yl)methyl)-1-oxoisoindolin-2- yl)dihydropyrimidine-2,4(1H,3H)-dione |
| GT- 0854 8 | 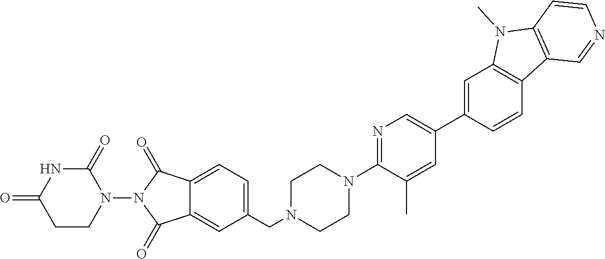 [SMILES: Cc1cc(-c2ccc3c4cnccc4n(C)c3c2)cnc1N1CCN(Cc2ccc3c(c2)C(=O)N(N2CCC(=O)NC2=O)C3=O)CC1] | 2-(2,4-dioxotetrahydropyrimidin-1(2H)-yl)-5- ((4-(3-methyl-5-(5-methyl-5H-pyrido[4,3- b]indol-7-yl)pyridin-2-yl)piperazin-1- yl)methyl)isoindoline-1,3-dione |
| GT- 0855 5 | 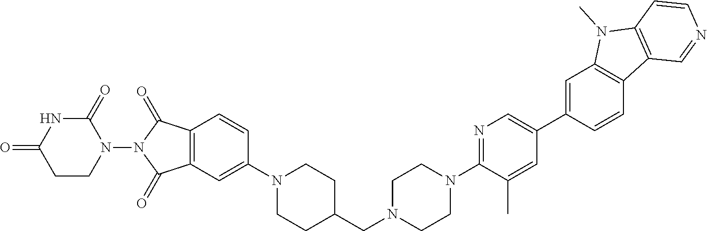 [SMILES: Cc1cc(-c2ccc3c4cnccc4n(C)c3c2)cnc1N1CCN(CC2CCN(c3ccc4c(c3)C(=O)N(N3CCC(=O)NC3=O)C4=O)CC2)CC1] | 2-(2,4-dioxotetrahydropyrimidin-1(2H)-yl)-5- (4-((4-(3-methyl-5-(5-methyl-5H-pyrido[4,3- b]indol-7-yl)pyridin-2-yl)piperazin-1- yl)methyl)piperidin-1-yl)isoindoline-1,3- dione |
| GT- 0856 3 | 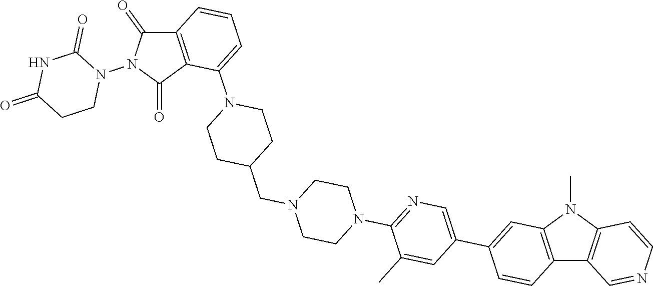 [SMILES: Cc1cc(-c2ccc3c4cnccc4n(C)c3c2)cnc1N1CCN(CC2CCN(c3cccc4c3C(=O)N(N3CCC(=O)NC3=O)C4=O)CC2)CC1] | 2-(2,4-dioxotetrahydropyrimidin-1(2H)-yl)-4- (4-((4-(3-methyl-5-(5-methyl-5H-pyrido[4,3- b]indol-7-yl)pyridin-2-yl)piperazin-1- yl)methyl)piperidin-1-yl)isoindoline-1,3- dione |
| GT- 0854 1 | 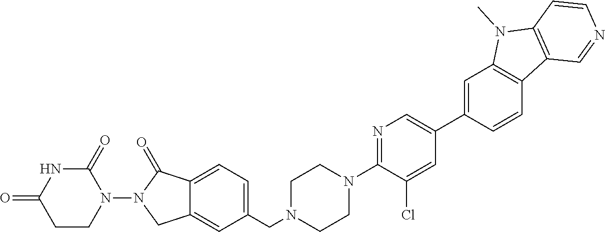 [SMILES: Cn1c2ccncc2c2ccc(-c3cnc(N4CCN(Cc5ccc6c(c5)CN(N5CCC(=O)NC5=O)C6=O)CC4)c(Cl)c3)cc21] | 1-(5-((4-(3-chloro-5-(5-methyl-5H- pyrido[4,3-b]indol-7-yl)pyridin-2- yl)piperazin-1-yl)methyl)-1-oxoisoindolin-2- yl)dihydropyrimidine-2,4(1H,3H)-dione |
| GT- 0854 9 | 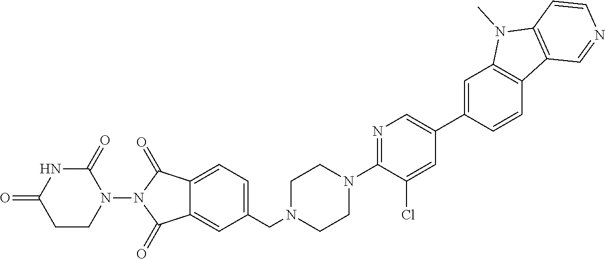 [SMILES: Cn1c2ccncc2c2ccc(-c3cnc(N4CCN(Cc5ccc6c(c5)C(=O)N(N5CCC(=O)NC5=O)C6=O)CC4)c(Cl)c3)cc21] | 5-((4-(3-chloro-5-(5-methyl-5H-pyrido[4,3- b]indol-7-yl)pyridin-2-yl)piperazin-1- yl)methyl)-2-(2,4-dioxotetrahydropyrimidin- 1(2H)-yl)isoindoline-1,3-dione |
| GT- 0855 6 | 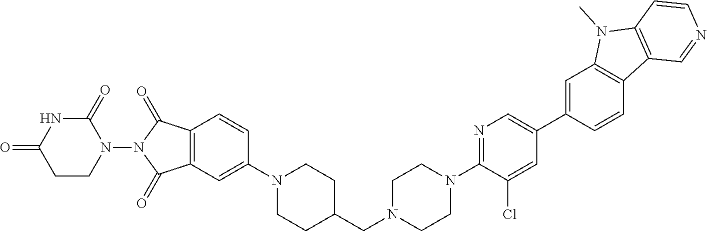 [SMILES: Cn1c2ccncc2c2ccc(-c3cnc(N4CCN(CC5CCN(c6ccc7c(c6)C(=O)N(N6CCC(=O)NC6=O)C7=O)CC5)CC4)c(Cl)c3)cc21] | 5-(4-((4-(3-chloro-5-(5-methyl-5H- pyrido[4,3-b]indol-7-yl)pyridin-2- yl)piperazin-1-yl)methyl)piperidin-1-yl)-2- (2,4-dioxotetrahydropyrimidin-1(2H)- yl)isoindoline-1,3-dione |
| GT- 0856 4 | 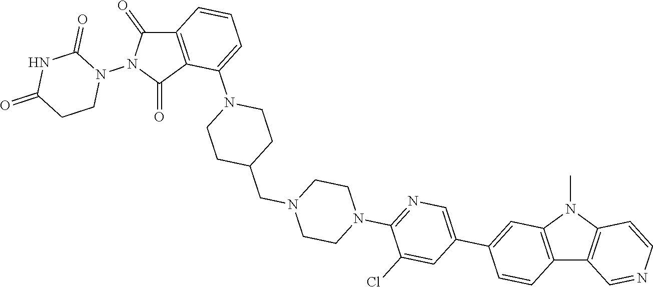 [SMILES: Cn1c2ccncc2c2ccc(-c3cnc(N4CCN(CC5CCN(c6cccc7c6C(=O)N(N6CCC(=O)NC6=O)C7=O)CC5)CC4)c(Cl)c3)cc21] | 4-(4-((4-(3-chloro-5-(5-methyl-5H- pyrido[4,3-b]indol-7-yl)pyridin-2- yl)piperazin-1-yl)methyl)piperidin-1-yl)-2- (2,4-dioxotetrahydropyrimidin-1(2H)- yl)isoindoline-1,3-dione |
| GT- 0856 5 | 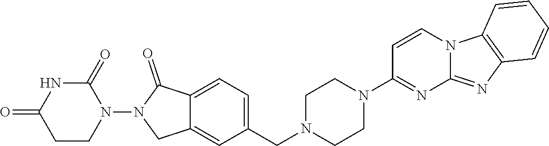 [SMILES: O=C1CCN(N2Cc3cc(CN4CCN(c5ccn6c(n5)nc5ccccc56)CC4)ccc3C2=O)C(=O)N1] | 1-(5-((4-(benzo[4,5]imidazo[1,2-a]pyrimidin- 2-yl)piperazin-1-yl)methyl)-1-oxoisoindolin- 2-yl)dihydropyrimidine-2,4(1H,3H)-dione |
| GT- 0856 7 | 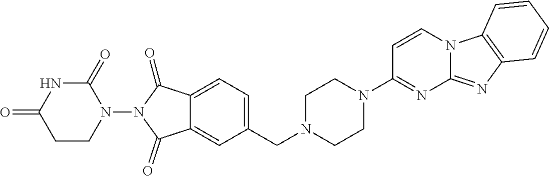 [SMILES: O=C1CCN(N2C(=O)c3ccc(CN4CCN(c5ccn6c(n5)nc5ccccc56)CC4)cc3C2=O)C(=O)N1] | 5-((4-(benzo[4,5]imidazo[1,2-a]pyrimidin-2- yl)piperazin-1-yl)methyl)-2-(2,4- dioxotetrahydropyrimidin-1(2H)- yl)isoindoline-1,3-dione |
| GT- 0857 0 | 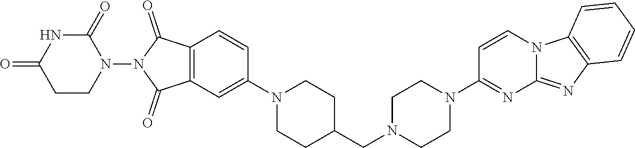 [SMILES: O=C1CCN(N2C(=O)c3ccc(N4CCC(CN5CCN(c6ccn7c(n6)nc6ccccc67)CC5)CC4)cc3C2=O)C(=O)N1] | 5-(4-((4-(benzo[4,5]imidazo[1,2-a]pyrimidin- 2-yl)piperazin-1-yl)methyl)piperidin-1-yl)-2- (2,4-dioxotetrahydropyrimidin-1(2H)- yl)isoindoline-1,3-dione |
| GT- 0857 1 | 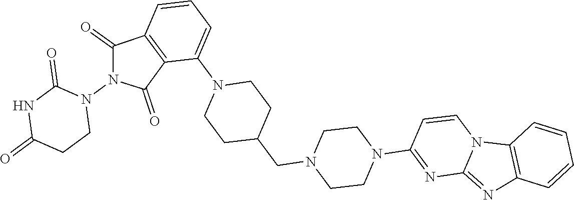 [SMILES: O=C1CCN(N2C(=O)c3cccc(N4CCC(CN5CCN(c6ccn7c(n6)nc6ccccc67)CC5)CC4)c3C2=O)C(=O)N1] | 4-(4-((4-(benzo[4,5]imidazo[1,2-a]pyrimidin- 2-yl)piperazin-1-yl)methyl)piperidin-1-yl)-2- (2,4-dioxotetrahydropyrimidin-1(2H)- yl)isoindoline-1,3-dione |
| GT- 0856 8 | 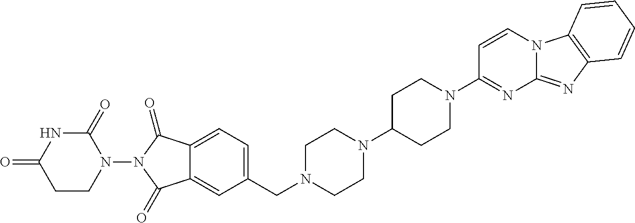 [SMILES: O=C1CCN(N2C(=O)c3ccc(CN4CCN(C5CCN(c6ccn7c(n6)nc6ccccc67)CC5)CC4)cc3C2=O)C(=O)N1] | 5-((4-(1-(benzo[4,5]imidazo[1,2-a]pyrimidin- 2-yl)piperidin-4-yl)piperazin-1-yl)methyl)-2- (2,4-dioxotetrahydropyrimidin-1(2H)- yl)isoindoline-1,3-dione |
| GT- 0856 6 | 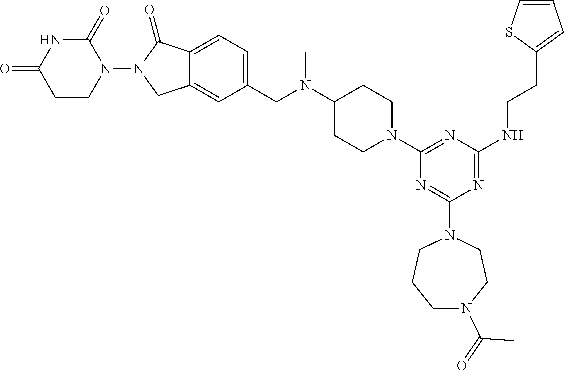 [SMILES: CC(=O)N1CCCN(c2nc(NCCc3cccs3)nc(N3CCC(N(C)Cc4ccc5c(c4)CN(N4CCC(=O)NC4=O)C5=O)CC3)n2)CC1] | 1-(5-(((1-(4-(4-acetyl-1,4-diazepan-1-yl)-6- ((2-(thiophen-2-yl)ethyl)amino)-1,3,5-triazin- 2-yl)piperidin-4-yl)(methyl)amino)methyl)-1- oxoisoindolin-2-yl)dihydropyrimidine- 2,4(1H,3H)-dione |
| GT- 0856 9 | 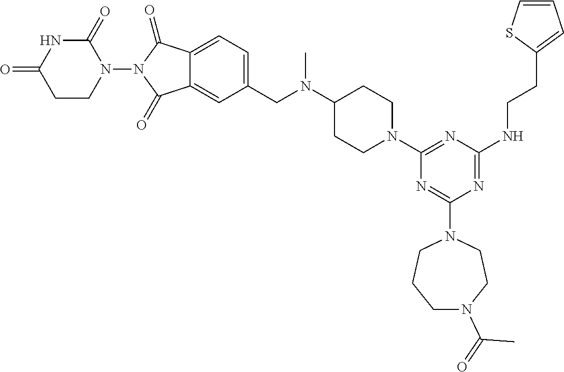 [SMILES: CC(=O)N1CCCN(c2nc(NCCc3cccs3)nc(N3CCC(N(C)Cc4ccc5c(c4)C(=O)N(N4CCC(=O)NC4=O)C5=O)CC3)n2)CC1] | 5-(((1-(4-(4-acetyl-1,4-diazepan-1-yl)-6-((2- (thiophen-2-yl)ethyl)amino)-1,3,5-triazin-2- yl)piperidin-4-yl)(methyl)amino)methyl)-2- (2,4-dioxotetrahydropyrimidin-1(2H)- yl)isoindoline-1,3-dione |
| GT- 0859 7 | 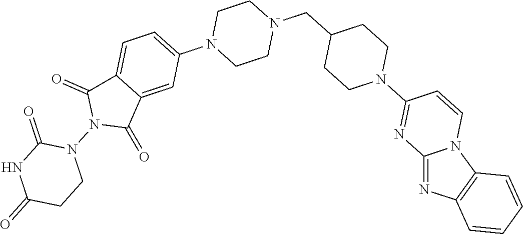 [SMILES: O=C1CCN(N2C(=O)c3ccc(N4CCN(CC5CCN(c6ccn7c(n6)nc6ccccc67)CC5)CC4)cc3C2=O)C(=O)N1] | 5-(4-((1-(benzo[4,5]imidazo[1,2-a]pyrimidin- 2-yl)piperidin-4-yl)methyl)piperazin-1-yl)-2- (2,4-dioxotetrahydropyrimidin-1(2H)- yl)isoindoline-1,3-dione |
| GT- 0860 5 | 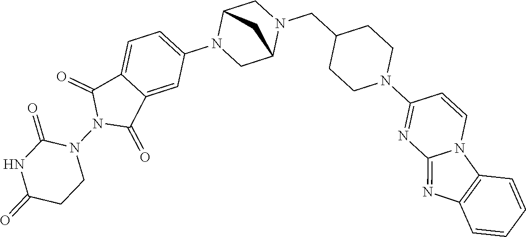 [SMILES: O=C1CCN(N2C(=O)c3ccc(N4C[C@@H]5C[C@H]4CN5CC4CCN(c5ccn6c(n5)nc5ccccc56)CC4)cc3C2=O)C(=O)N1] | 5-((1S,4S)-5-((1-(benzo[4,5]imidazo[1,2- a]pyrimidin-2-yl)piperidin-4-yl)methyl)-2,5- diazabicyclo[2.2.1]heptan-2-yl)-2-(2,4- dioxotetrahydropyrimidin-1(2H)- yl)isoindoline-1,3-dione |
| GT- 0860 6 | 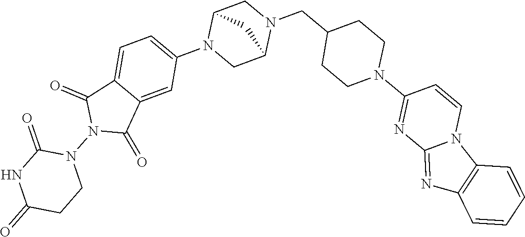 [SMILES: O=C1CCN(N2C(=O)c3ccc(N4C[C@H]5C[C@@H]4CN5CC4CCN(c5ccn6c(n5)nc5ccccc56)CC4)cc3C2=O)C(=O)N1] | 5-((1R,4R)-5-((1-(benzo[4,5]imidazo[1,2- a]pyrimidin-2-yl)piperidin-4-yl)methyl)-2,5- diazabicyclo[2.2.1]heptan-2-yl)-2-(2,4- dioxotetrahydropyrimidin-1(2H)- yl)isoindoline-1,3-dione |
| GT- 0860 7 | 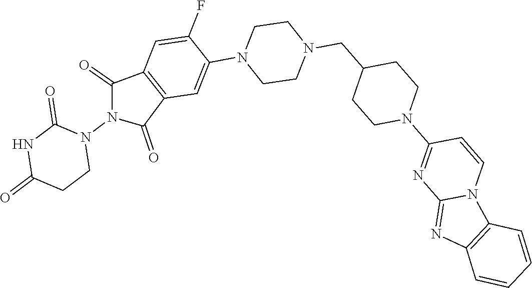 [SMILES: O=C1CCN(N2C(=O)c3cc(F)c(N4CCN(CC5CCN(c6ccn7c(n6)nc6ccccc67)CC5)CC4)cc3C2=O)C(=O)N1] | 5-(4-((1-(benzo[4,5]imidazo[1,2-a]pyrimidin- 2-yl)piperidin-4-yl)methyl)piperazin-1-yl)-2- (2,4-dioxotetrahydropyrimidin-1(2H)-yl)-6- fluoroisoindoline-1,3-dione |
| GT- 0860 8 | 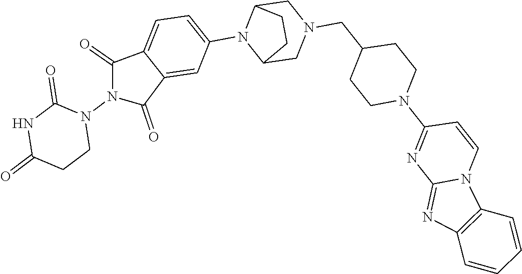 [SMILES: O=C1CCN(N2C(=O)c3ccc(N4C5CCC4CN(CC4CCN(c6ccn7c(n6)nc6ccccc67)CC4)C5)cc3C2=O)C(=O)N1] | 5-(3-((1-(benzo[4,5]imidazo[1,2-a]pyrimidin- 2-yl)piperidin-4-yl)methyl)-3,8- diazabicyclo[3.2.1]octan-8-yl)-2-(2,4- dioxotetrahydropyrimidin-1(2H)- yl)isoindoline-1,3-dione |
| GT- 0860 9 | 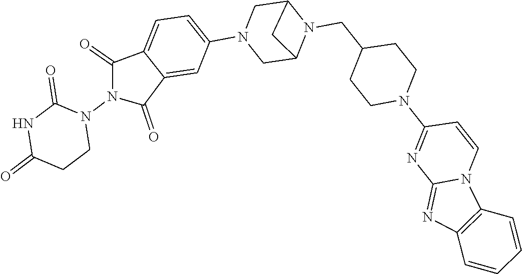 [SMILES: O=C1CCN(N2C(=O)c3ccc(N4CC5CC(C4)N5CC4CCN(c5ccn6c(n5)nc5ccccc56)CC4)cc3C2=O)C(=O)N1] | 5-(6-((1-(benzo[4,5]imidazo[1,2-a]pyrimidin- 2-yl)piperidin-4-yl)methyl)-3,6- diazabicyclo[3.1.1]heptan-3-yl)-2-(2,4- dioxotetrahydropyrimidin-1(2H)- yl)isoindoline-1,3-dione |
| GT- 0861 0 | 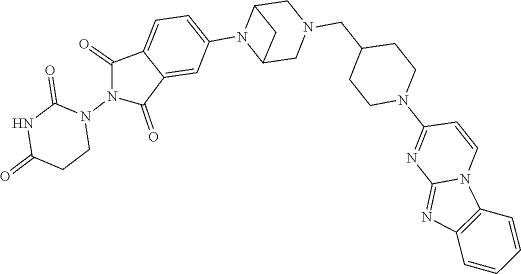 [SMILES: O=C1CCN(N2C(=O)c3ccc(N4C5CC4CN(CC4CCN(c6ccn7c(n6)nc6ccccc67)CC4)C5)cc3C2=O)C(=O)N1] | 5-(3-((1-(benzo[4,5]imidazo[1,2-a]pyrimidin- 2-yl)piperidin-4-yl)methyl)-3,6- diazabicyclo[3.1.1]heptan-6-yl)-2-(2,4- dioxotetrahydropyrimidin-1(2H)- yl)isoindoline-1,3-dione |
| GT- 0882 2 | 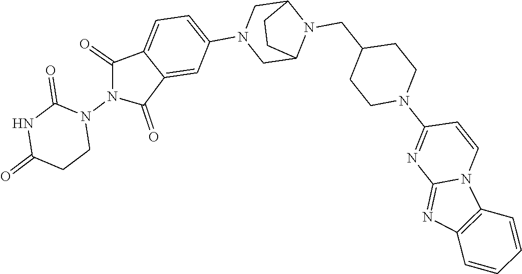 [SMILES: O=C1CCN(N2C(=O)c3ccc(N4CC5CCC(C4)N5CC4CCN(c5ccn6c(n5)nc5ccccc56)CC4)cc3C2=O)C(=O)N1] | 5-(8-((1-(benzo[4,5]imidazo[1,2-a]pyrimidin- 2-yl)piperidin-4-yl)methyl)-3,8- diazabicyclo[3.2.1]octan-3-yl)-2-(2,4- dioxotetrahydropyrimidin-1(2H)- yl)isoindoline-1,3-dione |
| GT- 0882 3 | 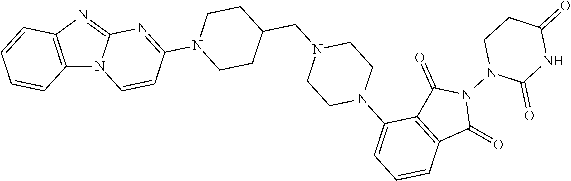 [SMILES: O=C1CCN(N2C(=O)c3cccc(N4CCN(CC5CCN(c6ccn7c(n6)nc6ccccc67)CC5)CC4)c3C2=O)C(=O)N1] | 4-(4-((1-(benzo[4,5]imidazo[1,2-a]pyrimidin- 2-yl)piperidin-4-yl)methyl)piperazin-1-yl)-2- (2,4-dioxotetrahydropyrimidin-1(2H)- yl)isoindoline-1,3-dione |
| GT- 0882 4 | 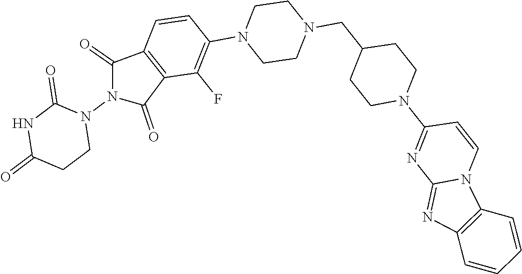 [SMILES: O=C1CCN(N2C(=O)c3ccc(N4CCN(CC5CCN(c6ccn7c(n6)nc6ccccc67)CC5)CC4)c(F)c3C2=O)C(=O)N1] | 5-(4-((1-(benzo[4,5]imidazo[1,2-a]pyrimidin- 2-yl)piperidin-4-yl)methyl)piperazin-1-yl)-2- (2,4-dioxotetrahydropyrimidin-1(2H)-yl)-4- fluoroisoindoline-1,3-dione |
| GT- 0882 5 | 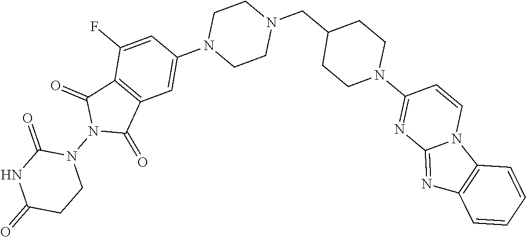 [SMILES: O=C1CCN(N2C(=O)c3cc(N4CCN(CC5CCN(c6ccn7c(n6)nc6ccccc67)CC5)CC4)cc(F)c3C2=O)C(=O)N1] | 6-(4-((1-(benzo[4,5]imidazo[1,2-a]pyrimidin- 2-yl)piperidin-4-yl)methyl)piperazin-1-yl)-2- (2,4-dioxotetrahydropyrimidin-1(2H)-yl)-4- fluoroisoindoline-1,3-dione |
| GT- 0882 6 | 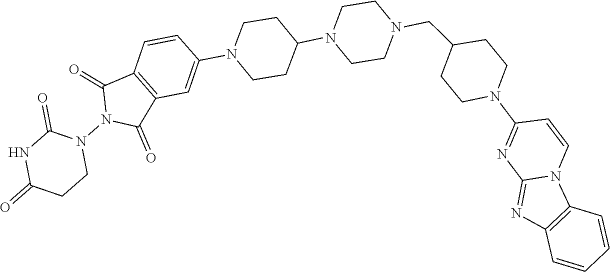 [SMILES: O=C1CCN(N2C(=O)c3ccc(N4CCC(N5CCN(CC6CCN(c7ccn8c(n7)nc7ccccc78)CC6)CC5)CC4)cc3C2=O)C(=O)N1] | 5-(4-(4-((1-(benzo[4,5]imidazo[1,2- a]pyrimidin-2-yl)piperidin-4- yl)methyl)piperazin-1-yl)piperidin-1-yl)-2- (2,4-dioxotetrahydropyrimidin-1(2H)- yl)isoindoline-1,3-dione |
| GT- 0882 7 | 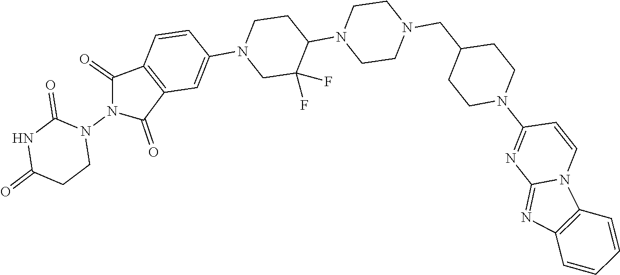 [SMILES: O=C1CCN(N2C(=O)c3ccc(N4CCC(N5CCN(CC6CCN(c7ccn8c(n7)nc7ccccc78)CC6)CC5)C(F)(F)C4)cc3C2=O)C(=O)N1] | 5-(4-(4-((1-(benzo[4,5]imidazo[1,2- a]pyrimidin-2-yl)piperidin-4- yl)methyl)piperazin-1-yl)-3,3- difluoropiperidin-1-yl)-2-(2,4- dioxotetrahydropyrimidin-1(2H)- yl)isoindoline-1,3-dione |
| GT- 0882 8 | 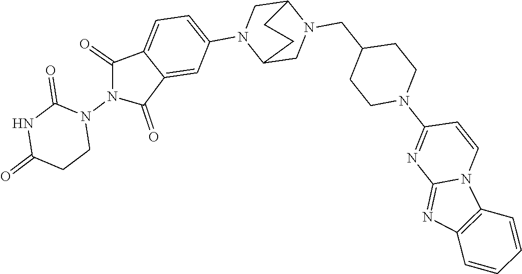 [SMILES: O=C1CCN(N2C(=O)c3ccc(N4CC5CCC4CN5CC4CCN(c5ccn6c(n5)nc5ccccc56)CC4)cc3C2=O)C(=O)N1] | 5-(5-((1-(benzo[4,5]imidazo[1,2-a]pyrimidin- 2-yl)piperidin-4-yl)methyl)-2,5- diazabicyclo[2.2.2]octan-2-yl)-2-(2,4- dioxotetrahydropyrimidin-1(2H)- yl)isoindoline-1,3-dione |
| GT- 0647 7 | 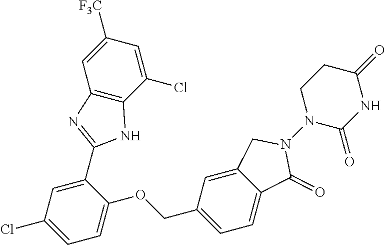 [SMILES: O=C1CCN(N2Cc3cc(COc4ccc(Cl)cc4-c4nc5cc(C(F)(F)F)cc(Cl)c5[nH]4)ccc3C2=O)C(=O)N1] | 1-(5-((4-chloro-2-(7-chloro-5- (trifluoromethyl)-1H-benzo[d]imidazol-2- yl)phenoxy)methyl)-1-oxoisoindolin-2- yl)dihydropyrimidine-2,4(1H,3H)-dione |
| GT- 0647 8 | 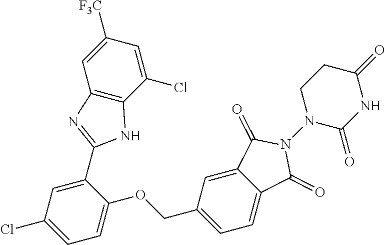 [SMILES: O=C1CCN(N2C(=O)c3ccc(COc4ccc(Cl)cc4-c4nc5cc(C(F)(F)F)cc(Cl)c5[nH]4)cc3C2=O)C(=O)N1] | 5-((4-chloro-2-(7-chloro-5-(trifluoromethyl)- 1H-benzo[d]imidazol-2-yl)phenoxy)methyl)- 2-(2,4-dioxotetrahydropyrimidin-1(2H)- yl)isoindoline-1,3-dione |
| GT- 0640 9 | 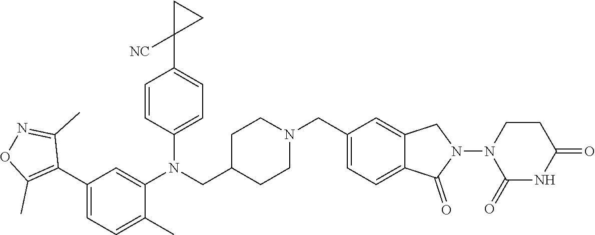 [SMILES: Cc1ccc(-c2c(C)noc2C)cc1N(CC1CCN(Cc2ccc3c(c2)CN(N2CCC(=O)NC2=O)C3=O)CC1)c1ccc(C2(C#N)CC2)cc1] | 1-(4-((5-(3,5-dimethylisoxazol-4-yl)-2- methylphenyl)((1-((2-(2,4- dioxotetrahydropyrimidin-1(2H)-yl)-1- oxoisoindolin-5-yl)methyl)piperidin-4- yl)methyl)amino)phenyl)cyclopropane-1- carbonitrile |
| GT- 0641 0 | 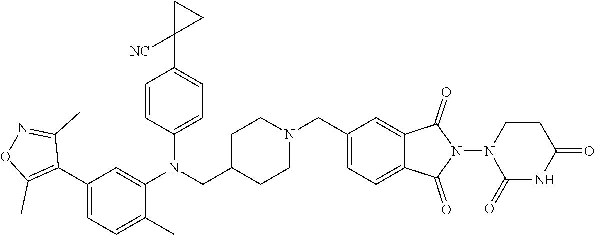 [SMILES: Cc1ccc(-c2c(C)noc2C)cc1N(CC1CCN(Cc2ccc3c(c2)C(=O)N(N2CCC(=O)NC2=O)C3=O)CC1)c1ccc(C2(C#N)CC2)cc1] | 1-(4-((5-(3,5-dimethylisoxazol-4-yl)-2- methylphenyl)((1-((2-(2,4- dioxotetrahydropyrimidin-1(2H)-yl)-1,3- dioxoisoindolin-5-yl)methyl)piperidin-4- yl)methyl)amino)phenyl)cyclopropane-1- carbonitrile |
| GT- 0641 8 | 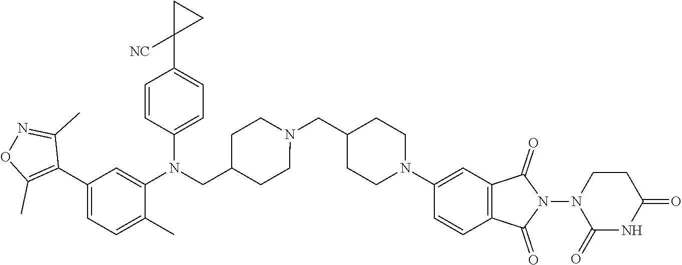 [SMILES: Cc1ccc(-c2c(C)noc2C)cc1N(CC1CCN(CC2CCN(c3ccc4c(c3)C(=O)N(N3CCC(=O)NC3=O)C4=O)CC2)CC1)c1ccc(C2(C#N)CC2)cc1] | 1-(4-((5-(3,5-dimethylisoxazol-4-yl)-2- methylphenyl)((1-((1-(2-(2,4- dioxotetrahydropyrimidin-1(2H)-yl)-1,3- dioxoisoindolin-5-yl)piperidin-4- yl)methyl)piperidin-4- yl)methyl)amino)phenyl)cyclopropane-1- carbonitrile |
| GT- 0641 9 | 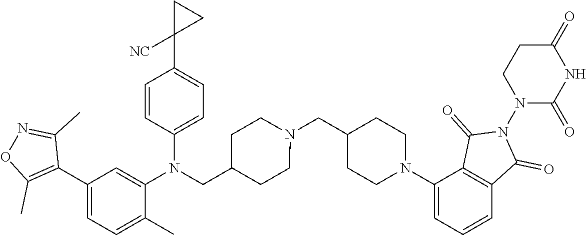 [SMILES: Cc1ccc(-c2c(C)noc2C)cc1N(CC1CCN(CC2CCN(c3cccc4c3C(=O)N(N3CCC(=O)NC3=O)C4=O)CC2)CC1)c1ccc(C2(C#N)CC2)cc1] | 1-(4-((5-(3,5-dimethylisoxazol-4-yl)-2- methylphenyl)((1-((1-(2-(2,4- dioxotetrahydropyrimidin-1(2H)-yl)-1,3- dioxoisoindolin-4-yl)piperidin-4- yl)methyl)piperidin-4- yl)methyl)amino)phenyl)cyclopropane-1- carbonitrile |
| GT- 0641 3 | 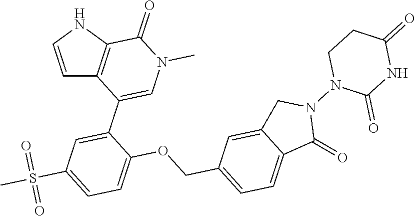 [SMILES: Cn1cc(-c2cc(S(C)(=O)=O)ccc2OCc2ccc3c(c2)CN(N2CCC(=O)NC2=O)C3=O)c2cc[nH]c2c1=O] | 1-(5-((2-(6-methyl-7-oxo-6,7-dihydro-1H- pyrrolo[2,3-c]pyridin-4-yl)-4- (methylsulfonyl)phenoxy)methyl)-1- oxoisoindolin-2-yl)dihydropyrimidine- 2,4(1H,3H)-dione |
| GT- 0582 1 | 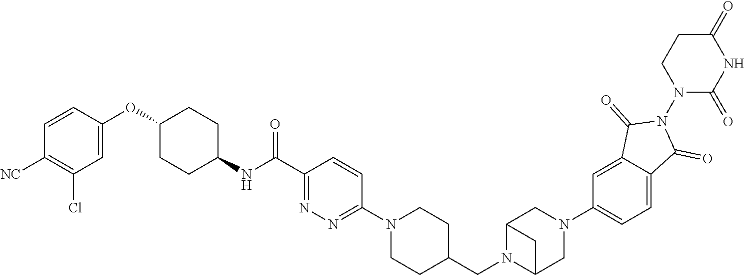 [SMILES: N#Cc1ccc(O[C@H]2CC[C@H](NC(=O)c3ccc(N4CCC(CN5C6CC5CN(c5ccc7c(c5)C(=O)N(N5CCC(=O)NC5=O)C7=O)C6)CC4)nn3)CC2)cc1Cl] | N-((1r,4r)-4-(3-chloro-4- cyanophenoxy)cyclohexyl)-6-(4-((3-(2-(2,4- dioxotetrahydropyrimidin-1(2H)-yl)-1,3- dioxoisoindolin-5-yl)-3,6- diazabicyclo[3.1.1]heptan-6- yl)methyl)piperidin-1-yl)pyridazine-3- carboxamide |
| GT- 0582 4 | 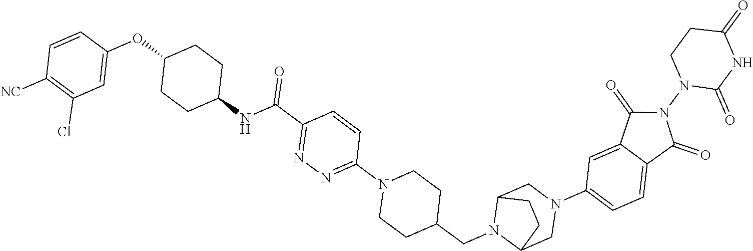 [SMILES: N#Cc1ccc(O[C@H]2CC[C@H](NC(=O)c3ccc(N4CCC(CN5C6CCC5CN(c5ccc7c(c5)C(=O)N(N5CCC(=O)NC5=O)C7=O)C6)CC4)nn3)CC2)cc1Cl] | N-((1r,4r)-4-(3-chloro-4- cyanophenoxy)cyclohexyl)-6-(4-((3-(2-(2,4- dioxotetrahydropyrimidin-1(2H)-yl)-1,3- dioxoisoindolin-5-yl)-3,8- diazabicyclo[3.2.1]octan-8- yl)methyl)piperidin-1-yl)pyridazine-3- carboxamide |
| GT- 0582 9 | 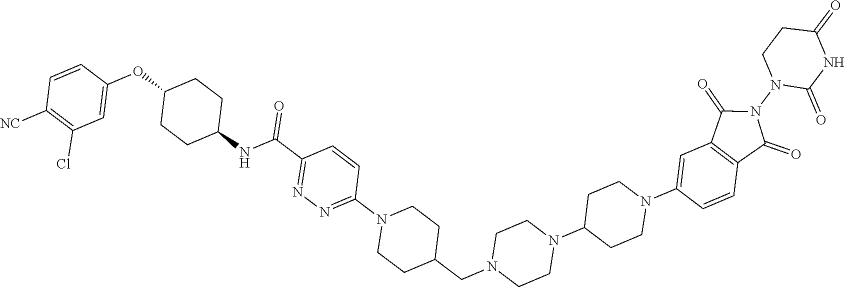 [SMILES: N#Cc1ccc(O[C@H]2CC[C@H](NC(=O)c3ccc(N4CCC(CN5CCN(C6CCN(c7ccc8c(c7)C(=O)N(N7CCC(=O)NC7=O)C8=O)CC6)CC5)CC4)nn3)CC2)cc1Cl] | N-((1r,4r)-4-(3-chloro-4- cyanophenoxy)cyclohexyl)-6-(4-((4-(1-(2- (2,4-dioxotetrahydropyrimidin-1(2H)-yl)-1,3- dioxoisoindolin-5-yl)piperidin-4-yl)piperazin- 1-yl)methyl)piperidin-1-yl)pyridazine-3- carboxamide |
| GT- 0583 2 | 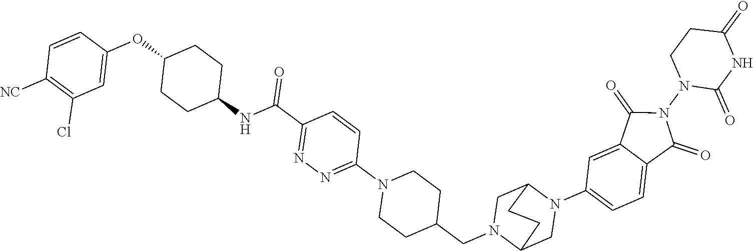 [SMILES: N#Cc1ccc(O[C@H]2CC[C@H](NC(=O)c3ccc(N4CCC(CN5CC6CCC5CN6c5ccc6c(c5)C(=O)N(N5CCC(=O)NC5=O)C6=O)CC4)nn3)CC2)cc1Cl] | N-((1r,4r)-4-(3-chloro-4- cyanophenoxy)cyclohexyl)-6-(4-((5-(2-(2,4- dioxotetrahydropyrimidin-1(2H)-yl)-1,3- dioxoisoindolin-5-yl)-2,5- diazabicyclo[2.2.2]octan-2- yl)methyl)piperidin-1-yl)pyridazine-3- carboxamide |
| GT- 0582 3 | 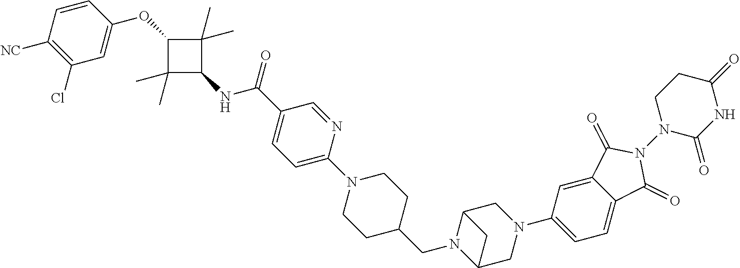 [SMILES: CC1(C)[C@H](NC(=O)c2ccc(N3CCC(CN4C5CC4CN(c4ccc6c(c4)C(=O)N(N4CCC(=O)NC4=O)C6=O)C5)CC3)nc2)C(C)(C)[C@H]1Oc1ccc(C#N)c(Cl)c1] | N-((1r,3r)-3-(3-chloro-4-cyanophenoxy)- 2,2,4,4-tetramethylcyclobutyl)-6-(4-((3-(2- (2,4-dioxotetrahydropyrimidin-1(2H)-yl)-1,3- dioxoisoindolin-5-yl)-3,6- diazabicyclo[3.1.1]heptan-6- yl)methyl)piperidin-1-yl)nicotinamide |
| GT- 0582 6 | 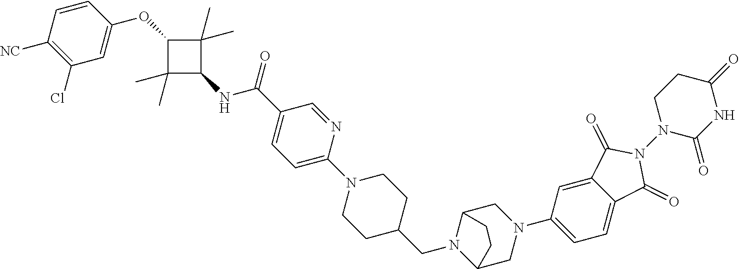 [SMILES: CC1(C)[C@H](NC(=O)c2ccc(N3CCC(CN4C5CCC4CN(c4ccc6c(c4)C(=O)N(N4CCC(=O)NC4=O)C6=O)C5)CC3)nc2)C(C)(C)[C@H]1Oc1ccc(C#N)c(Cl)c1] | N-((1r,3r)-3-(3-chloro-4-cyanophenoxy)- 2,2,4,4-tetramethylcyclobutyl)-6-(4-((3-(2- (2,4-dioxotetrahydropyrimidin-1(2H)-yl)-1,3- dioxoisoindolin-5-yl)-3,8- diazabicyclo[3.2.1]octan-8- yl)methyl)piperidin-1-yl)nicotinamide |
| GT- 0582 8 | 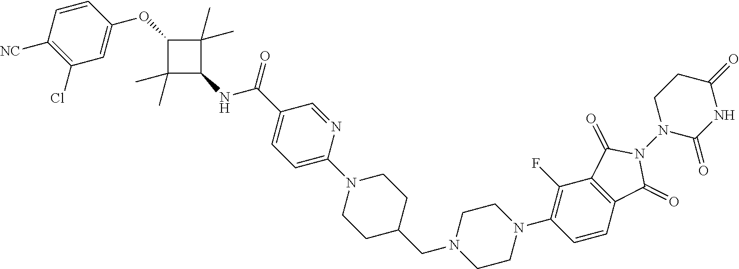 [SMILES: CC1(C)[C@H](NC(=O)c2ccc(N3CCC(CN4CCN(c5ccc6c(c5F)C(=O)N(N5CCC(=O)NC5=O)C6=O)CC4)CC3)nc2)C(C)(C)[C@H]1Oc1ccc(C#N)c(Cl)c1] | N-((1r,3r)-3-(3-chloro-4-cyanophenoxy)- 2,2,4,4-tetramethylcyclobutyl)-6-(4-((4-(2- (2,4-dioxotetrahydropyrimidin-1(2H)-yl)-4- fluoro-1,3-dioxoisoindolin-5-yl)piperazin-1- yl)methyl)piperidin-1-yl)nicotinamide |
| GT- 0583 0 | 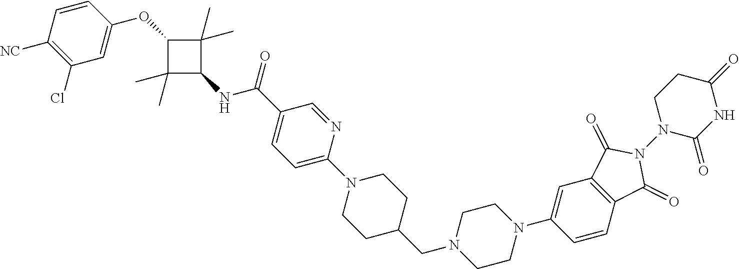 [SMILES: CC1(C)[C@H](NC(=O)c2ccc(N3CCC(CN4CCN(c5ccc6c(c5)C(=O)N(N5CCC(=O)NC5=O)C6=O)CC4)CC3)nc2)C(C)(C)[C@H]1Oc1ccc(C#N)c(Cl)c1] | N-((1r,3r)-3-(3-chloro-4-cyanophenoxy)- 2,2,4,4-tetramethylcyclobutyl)-6-(4-((4-(1-(2- (2,4-dioxotetrahydropyrimidin-1(2H)-yl)-1,3- dioxoisoindolin-5-yl)piperidin-4-yl)piperazin- 1-yl)methyl)piperidin-1-yl)nicotinamide |
| GT- 0583 4 | 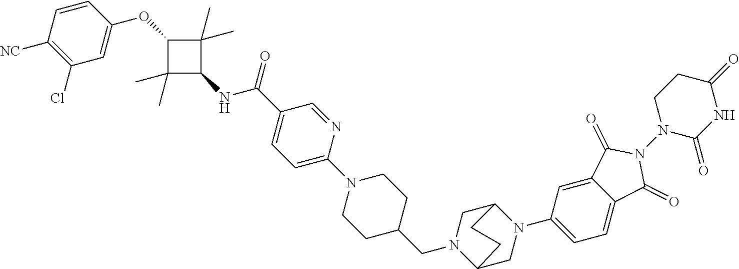 [SMILES: CC1(C)[C@H](NC(=O)c2ccc(N3CCC(CN4CC5CCC4CN5c4ccc5c(c4)C(=O)N(N4CCC(=O)NC4=O)C5=O)CC3)nc2)C(C)(C)[C@H]1Oc1ccc(C#N)c(Cl)c1] | N-((1r,3r)-3-(3-chloro-4-cyanophenoxy)- 2,2,4,4-tetramethylcyclobutyl)-6-(4-((5-(2- (2,4-dioxotetrahydropyrimidin-1(2H)-yl)-1,3- dioxoisoindolin-5-yl)-2,5- diazabicyclo[2.2.2]octan-2- yl)methyl)piperidin-1-yl)nicotinamide |
| GT- 0857 2 | 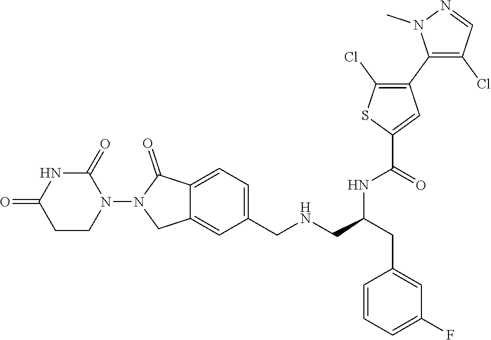 [SMILES: Cn1ncc(Cl)c1-c1cc(C(=O)N[C@H](CNCc2ccc3c(c2)CN(N2CCC(=O)NC2=O)C3=O)Cc2cccc(F)c2)sc1Cl] | (S)-5-chloro-4-(4-chloro-1-methyl-1H- pyrazol-5-yl)-N-(1-(((2-(2,4- dioxotetrahydropyrimidin-1(2H)-yl)-1- oxoisoindolin-5-yl)methyl)amino)-3-(3- fluorophenyl)propan-2-yl)thiophene-2- carboxamide |
| GT- 0857 3 | 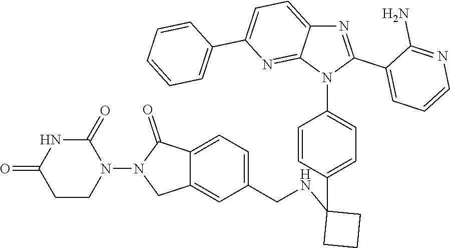 [SMILES: Nc1ncccc1-c1nc2ccc(-c3ccccc3)nc2n1-c1ccc(C2(NCc3ccc4c(c3)CN(N3CCC(=O)NC3=O)C4=O)CCC2)cc1] | 1-(5-(((1-(4-(2-(2-aminopyridin-3-yl)-5- phenyl-3H-imidazo[4,5-b]pyridin-3- yl)phenyl)cyclobutyl)amino)methyl)-1- oxoisoindolin-2-yl)dihydropyrimidine- 2,4(1H,3H)-dione |
| GT- 0857 4 | 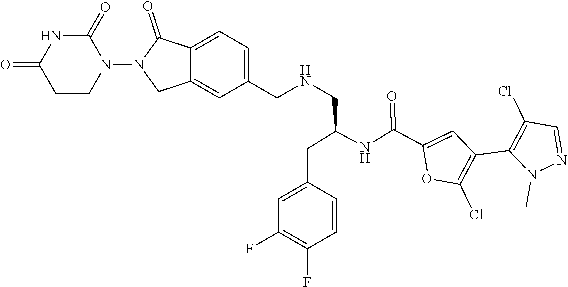 [SMILES: Cn1ncc(Cl)c1-c1cc(C(=O)N[C@H](CNCc2ccc3c(c2)CN(N2CCC(=O)NC2=O)C3=O)Cc2ccc(F)c(F)c2)oc1Cl] | (S)-5-chloro-4-(4-chloro-1-methyl-1H- pyrazol-5-yl)-N-(1-(3,4-difluorophenyl)-3- (((2-(2,4-dioxotetrahydropyrimidin-1(2H)- yl)-1-oxoisoindolin-5- yl)methyl)amino)propan-2-yl)furan-2- carboxamide |
| GT- 0857 5 | 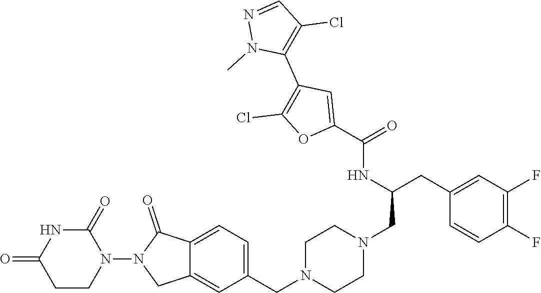 [SMILES: Cn1ncc(Cl)c1-c1cc(C(=O)N[C@@H](Cc2ccc(F)c(F)c2)CN2CCN(Cc3ccc4c(c3)CN(N3CCC(=O)NC3=O)C4=O)CC2)oc1Cl] | (S)-5-chloro-4-(4-chloro-1-methyl-1H- pyrazol-5-yl)-N-(1-(3,4-difluorophenyl)-3-(4- ((2-(2,4-dioxotetrahydropyrimidin-1(2H)-yl)- 1-oxoisoindolin-5-yl)methyl)piperazin-1- yl)propan-2-yl)furan-2-carboxamide |
| GT- 0857 6 | 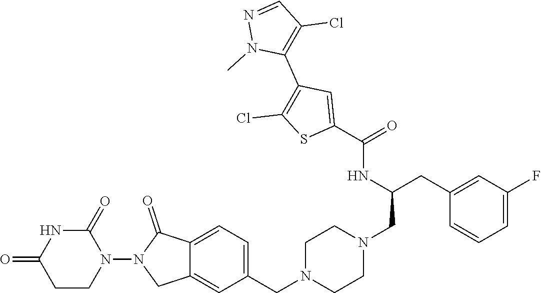 [SMILES: Cn1ncc(Cl)c1-c1cc(C(=O)N[C@@H](Cc2cccc(F)c2)CN2CCN(Cc3ccc4c(c3)CN(N3CCC(=O)NC3=O)C4=O)CC2)sc1Cl] | (S)-5-chloro-4-(4-chloro-1-methyl-1H- pyrazol-5-yl)-N-(1-(4-((2-(2,4- dioxotetrahydropyrimidin-1(2H)-yl)-1- oxoisoindolin-5-yl)methyl)piperazin-1-yl)-3- (3-fluorophenyl)propan-2-yl)thiophene-2- carboxamide |
| GT- 0857 7 | 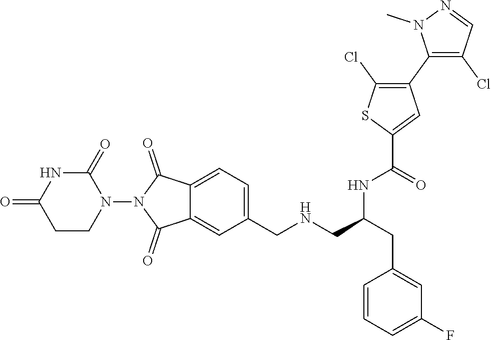 [SMILES: Cn1ncc(Cl)c1-c1cc(C(=O)N[C@H](CNCc2ccc3c(c2)C(=O)N(N2CCC(=O)NC2=O)C3=O)Cc2cccc(F)c2)sc1Cl] | (S)-5-chloro-4-(4-chloro-1-methyl-1H- pyrazol-5-yl)-N-(1-(((2-(2,4- dioxotetrahydropyrimidin-1(2H)-yl)-1,3- dioxoisoindolin-5-yl)methyl)amino)-3-(3- fluorophenyl)propan-2-yl)thiophene-2- carboxamide |
| GT- 0857 8 | 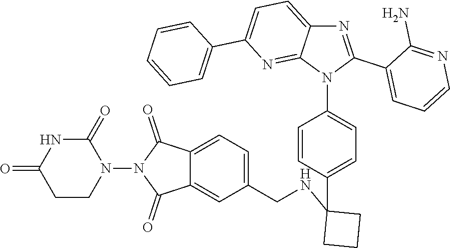 [SMILES: Nc1ncccc1-c1nc2ccc(-c3ccccc3)nc2n1-c1ccc(C2(NCc3ccc4c(c3)C(=O)N(N3CCC(=O)NC3=O)C4=O)CCC2)cc1] | 5-(((1-(4-(2-(2-aminopyridin-3-yl)-5-phenyl- 3H-imidazo[4,5-b]pyridin-3- yl)phenyl)cyclobutyl)amino)methyl)-2-(2,4- dioxotetrahydropyrimidin-1(2H)- yl)isoindoline-1,3-dione |
| GT- 0857 9 | 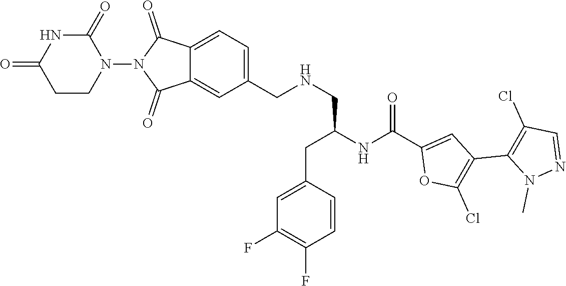 [SMILES: Cn1ncc(Cl)c1-c1cc(C(=O)N[C@H](CNCc2ccc3c(c2)C(=O)N(N2CCC(=O)NC2=O)C3=O)Cc2ccc(F)c(F)c2)oc1Cl] | (S)-5-chloro-4-(4-chloro-1-methyl-1H- pyrazol-5-yl)-N-(1-(3,4-difluorophenyl)-3- (((2-(2,4-dioxotetrahydropyrimidin-1(2H)- yl)-1,3-dioxoisoindolin-5- yl)methyl)amino)propan-2-yl)furan-2- carboxamide |
| GT- 0858 0 | 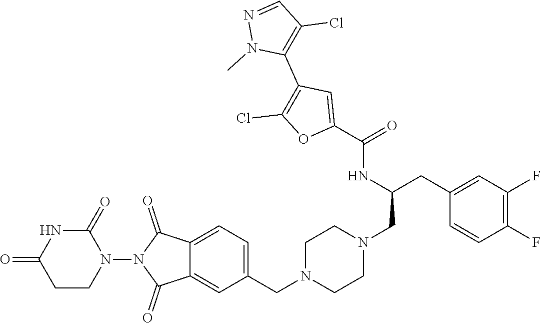 [SMILES: Cn1ncc(Cl)c1-c1cc(C(=O)N[C@@H](Cc2ccc(F)c(F)c2)CN2CCN(Cc3ccc4c(c3)C(=O)N(N3CCC(=O)NC3=O)C4=O)CC2)oc1Cl] | (S)-5-chloro-4-(4-chloro-1-methyl-1H- pyrazol-5-yl)-N-(1-(3,4-difluorophenyl)-3-(4- ((2-(2,4-dioxotetrahydropyrimidin-1(2H)-yl)- 1,3-dioxoisoindolin-5-yl)methyl)piperazin-1- yl)propan-2-yl)furan-2-carboxamide |
| GT- 0858 1 | 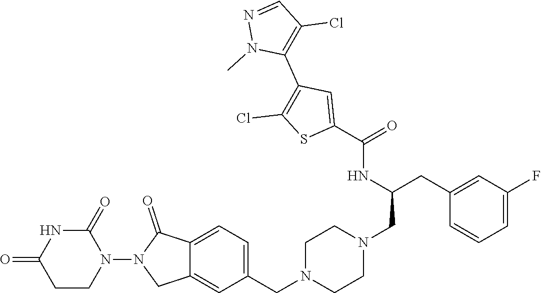 [SMILES: Cn1ncc(Cl)c1-c1cc(C(=O)N[C@@H](Cc2cccc(F)c2)CN2CCN(Cc3ccc4c(c3)CN(N3CCC(=O)NC3=O)C4=O)CC2)sc1Cl] | (S)-5-chloro-4-(4-chloro-1-methyl-1H- pyrazol-5-yl)-N-(1-(4-((2-(2,4- dioxotetrahydropyrimidin-1(2H)-yl)-1,3- dioxoisoindolin-5-yl)methyl)piperazin-1-yl)- 3-(3-fluorophenyl)propan-2-yl)thiophene-2- carboxamide |
| GT- 0858 2 | 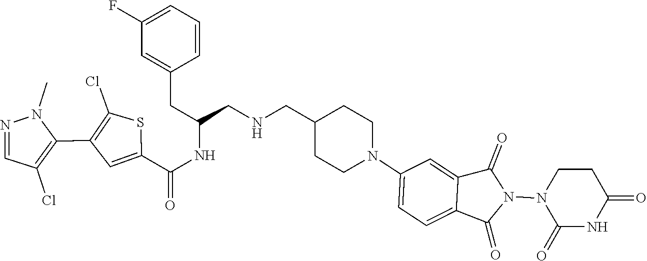 | (S)-5-chloro-4-(4-chloro-1-methyl-1H- pyrazol-5-yl)-N-(1-(((1-(2-(2,4- dioxotetrahydropyrimidin-1(2H)-yl)-1,3- dioxoisoindolin-5-yl)piperidin-4- yl)methyl)amino)-3-(3-fluorophenyl)propan- 2-yl)thiophene-2-carboxamide |
| GT- 0858 3 | 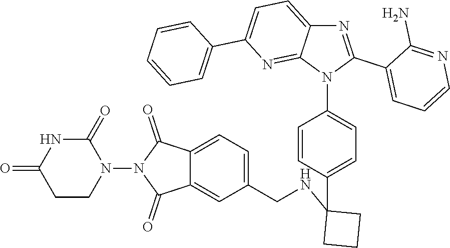 [SMILES: Nc1ncccc1-c1nc2ccc(-c3ccccc3)nc2n1-c1ccc(C2(NCc3ccc4c(c3)C(=O)N(N3CCC(=O)NC3=O)C4=O)CCC2)cc1] | 5-(4-(((1-(4-(2-(2-aminopyridin-3-yl)-5- phenyl-3H-imidazo[4,5-b]pyridin-3- yl)phenyl)cyclobutyl)amino)methyl)piperidin- 1-yl)-2-(2,4-dioxotetrahydropyrimidin- 1(2H)-yl)isoindoline-1,3-dione |
| GT- 0858 4 | 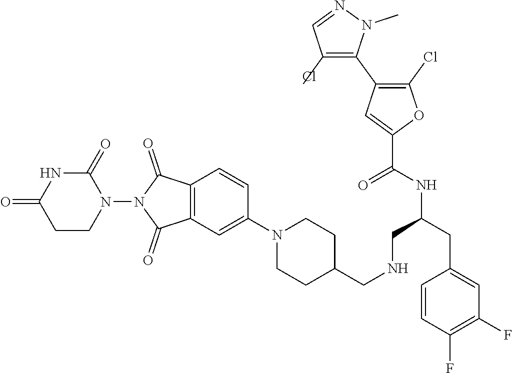 [SMILES: Cc1cnn(C)c1-c1cc(C(=O)N[C@H](CNCC2CCN(c3ccc4c(c3)C(=O)N(N3CCC(=O)NC3=O)C4=O)CC2)Cc2ccc(F)c(F)c2)oc1Cl ; Cl] | (S)-5-chloro-4-(4-chloro-1-methyl-1H- pyrazol-5-yl)-N-(1-(3,4-difluorophenyl)-3- (((1-(2-(2,4-dioxotetrahydropyrimidin-1(2H)- yl)-1,3-dioxoisoindolin-5-yl)piperidin-4- yl)methyl)amino)propan-2-yl)furan-2- carboxamide |
| GT- 0858 5 | 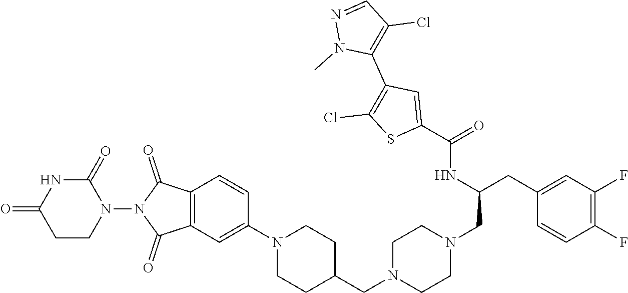 [SMILES: Cn1ncc(Cl)c1-c1cc(C(=O)N[C@@H](Cc2ccc(F)c(F)c2)CN2CCN(CC3CCN(c4ccc5c(c4)C(=O)N(N4CCC(=O)NC4=O)C5=O)CC3)CC2)sc1Cl] | (S)-5-chloro-4-(4-chloro-1-methyl-1H- pyrazol-5-yl)-N-(1-(3,4-difluorophenyl)-3-(4- ((1-(2-(2,4-dioxotetrahydropyrimidin-1(2H)- yl)-1,3-dioxoisoindolin-5-yl)piperidin-4- yl)methyl)piperazin-1-yl)propan-2-yl)furan- 2-carboxamide |
| GT- 0858 6 | 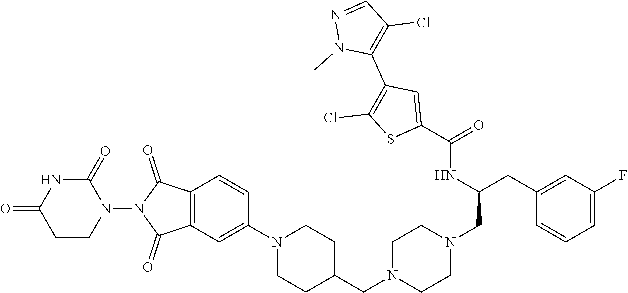 [SMILES: Cn1ncc(Cl)c1-c1cc(C(=O)N[C@@H](Cc2cccc(F)c2)CN2CCN(CC3CCN(c4ccc5c(c4)C(=O)N(N4CCC(=O)NC4=O)C5=O)CC3)CC2)sc1Cl] | (S)-5-chloro-4-(4-chloro-1-methyl-1H- pyrazol-5-yl)-N-(1-(4-((1-(2-(2,4- dioxotetrahydropyrimidin-1(2H)-yl)-1,3- dioxoisoindolin-5-yl)piperidin-4- yl)methyl)piperazin-1-yl)-3-(3- fluorophenyl)propan-2-yl)thiophene-2- carboxamide |
| GT- 0858 7 | 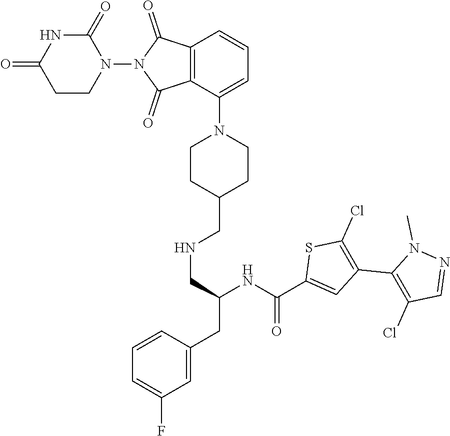 [SMILES: Cn1ncc(Cl)c1-c1cc(C(=O)N[C@H](CNCC2CCN(c3cccc4c3C(=O)N(N3CCC(=O)NC3=O)C4=O)CC2)Cc2cccc(F)c2)sc1Cl] | (S)-5-chloro-4-(4-chloro-1-methyl-1H- pyrazol-5-yl)-N-(1-(((1-(2-(2,4- dioxotetrahydropyrimidin-1(2H)-yl)-1,3- dioxoisoindolin-4-yl)piperidin-4- yl)methyl)amino)-3-(3-fluorophenyl)propan- 2-yl)thiophene-2-carboxamide |
| GT- 0858 8 | 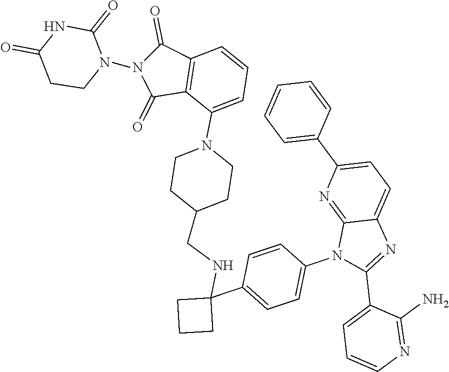 [SMILES: Nc1ncccc1-c1nc2ccc(-c3ccccc3)nc2n1-c1ccc(C2(NCC3CCN(c4cccc5c4C(=O)N(N4CCC(=O)NC4=O)C5=O)CC3)CCC2)cc1] | 4-(4-(((1-(4-(2-(2-aminopyridin-3-yl)-5- phenyl-3H-imidazo[4,5-b]pyridin-3- yl)phenyl)cyclobutyl)amino)methyl)piperidin- 1-yl)-2-(2,4-dioxotetrahydropyrimidin- 1(2H)-yl)isoindoline-1,3-dione |
| GT- 0858 9 | 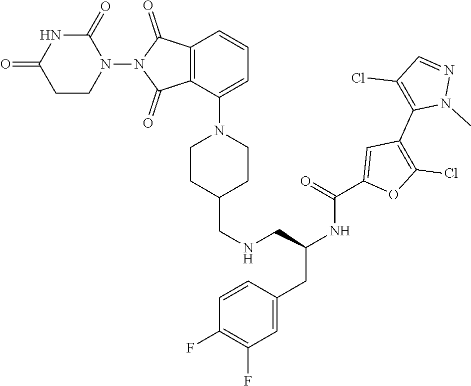 [SMILES: Cn1ncc(Cl)c1-c1cc(C(=O)N[C@H](CNCC2CCN(c3cccc4c3C(=O)N(N3CCC(=O)NC3=O)C4=O)CC2)Cc2ccc(F)c(F)c2)oc1Cl] | (S)-5-chloro-4-(4-chloro-1-methyl-1H- pyrazol-5-yl)-N-(1-(3,4-difluorophenyl)-3- (((1-(2-(2,4-dioxotetrahydropyrimidin-1(2H)- yl)-1,3-dioxoisoindolin-4-yl)piperidin-4- yl)methyl)amino)propan-2-yl)furan-2- carboxamide |
| GT- 0859 0 | 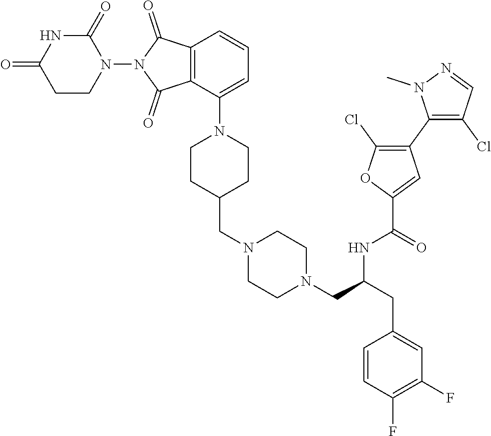 [SMILES: Cn1ncc(Cl)c1-c1cc(C(=O)N[C@@H](Cc2ccc(F)c(F)c2)CN2CCN(CC3CCN(c4cccc5c4C(=O)N(N4CCC(=O)NC4=O)C5=O)CC3)CC2)oc1Cl] | (S)-5-chloro-4-(4-chloro-1-methyl-1H- pyrazol-5-yl)-N-(1-(3,4-difluorophenyl)-3-(4- ((1-(2-(2,4-dioxotetrahydropyrimidin-1(2H)- yl)-1,3-dioxoisoindolin-4-yl)piperidin-4- yl)methyl)piperazin-1-yl)propan-2-yl)furan- 2-carboxamide |
| GT- 0859 1 | 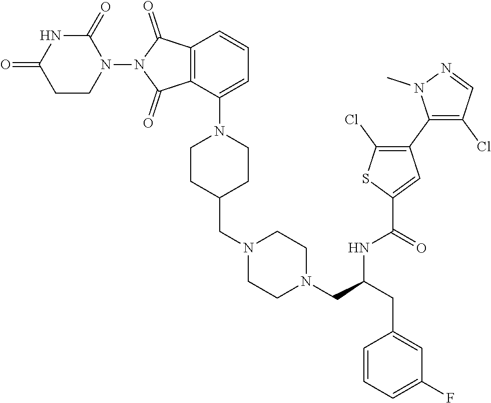 [SMILES: Cn1ncc(Cl)c1-c1cc(C(=O)N[C@@H](Cc2cccc(F)c2)CN2CCN(CC3CCN(c4cccc5c4C(=O)N(N4CCC(=O)NC4=O)C5=O)CC3)CC2)sc1Cl] | (S)-5-chloro-4-(4-chloro-1-methyl-1H- pyrazol-5-yl)-N-(1-(4-((1-(2-(2,4- dioxotetrahydropyrimidin-1(2H)-yl)-1,3- dioxoisoindolin-4-yl)piperidin-4- yl)methyl)piperazin-1-yl)-3-(3- fluorophenyl)propan-2-yl)thiophene-2- carboxamide |
| GT- 0840 3 | 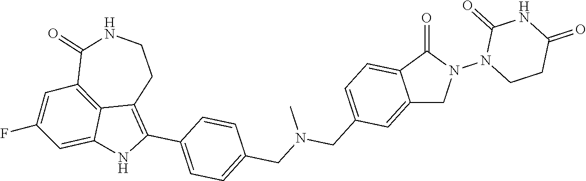 [SMILES: CN(Cc1ccc(-c2[nH]c3cc(F)cc4c3c2CCNC4=O)cc1)Cc1ccc2c(c1)CN(N1CCC(=O)NC1=O)C2=O] | 1-(5-(((4-(8-fluoro-1-oxo-2,3,4,6-tetrahydro- 1H-azepino[5,4,3-cd]indol-5- yl)benzyl)(methyl)amino)methyl)-1- oxoisoindolin-2-yl)dihydropyrimidine- 2,4(1H,3H)-dione |
| GT- 0840 4 | 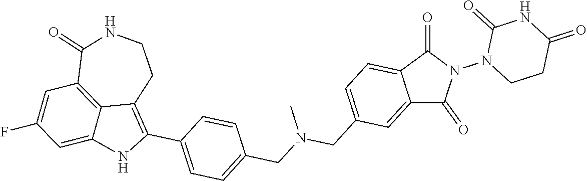 [SMILES: CN(Cc1ccc(-c2[nH]c3cc(F)cc4c3c2CCNC4=O)cc1)Cc1ccc2c(c1)C(=O)N(N1CCC(=O)NC1=O)C2=O] | 2-(2,4-dioxotetrahydropyrimidin-1(2H)-yl)-5- (((4-(8-fluoro-1-oxo-2,3,4,6-tetrahydro-1H- azepino[5,4,3-cd]indol-5- yl)benzyl)(methyl)amino)methyl)isoindoline- 1,3-dione |
| GT- 0840 5 | 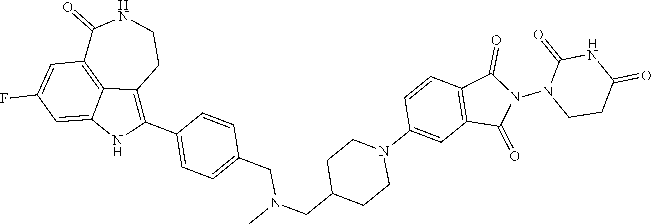 [SMILES: CN(Cc1ccc(-c2[nH]c3cc(F)cc4c3c2CCNC4=O)cc1)CC1CCN(c2ccc3c(c2)C(=O)N(N2CCC(=O)NC2=O)C3=O)CC1] | 2-(2,4-dioxotetrahydropyrimidin-1(2H)-yl)-5- (4-(((4-(8-fluoro-1-oxo-2,3,4,6-tetrahydro- 1H-azepino[5,4,3-cd]indol-5- yl)benzyl)(methyl)amino)methyl)piperidin-1- yl)isoindoline-1,3-dione |
| GT- 0840 6 | 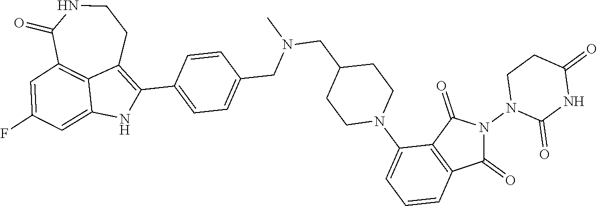 [SMILES: CN(Cc1ccc(-c2[nH]c3cc(F)cc4c3c2CCNC4=O)cc1)CC1CCN(c2cccc3c2C(=O)N(N2CCC(=O)NC2=O)C3=O)CC1] | 2-(2,4-dioxotetrahydropyrimidin-1(2H)-yl)-4- (4-(((4-(8-fluoro-1-oxo-2,3,4,6-tetrahydro- 1H-azepino[5,4,3-cd]indol-5- yl)benzyl)(methyl)amino)methyl)piperidin-1- yl)isoindoline-1,3-dione |
| GT- 0897 3 | 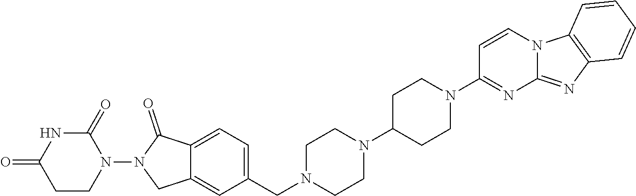 [SMILES: O=C1CCN(N2Cc3cc(CN4CCN(C5CCN(c6ccn7c(n6)nc6ccccc67)CC5)CC4)ccc3C2=O)C(=O)N1] | 1-(5-((4-(1-(benzo[4,5]imidazo[1,2- a]pyrimidin-2-yl)piperidin-4-yl)piperazin-1- yl)methyl)-1-oxoisoindolin-2- yl)dihydropyrimidine-2,4(1H,3H)-dione |

### Compound of Formula (II)

The present disclosure provides a compound of Formula (II) or salts (including pharmaceutically acceptable salts), stereoisomers (including enantiomers), solvates, isotopically enriched analogs, prodrugs, or polymorphs thereof:

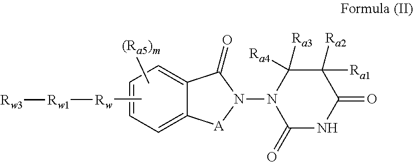

wherein A represents CO, CH₂, or CD₂;
Rₐ₁, Rₐ₂, Rₐ₃, and Rₐ₄ are the same or different and independently represent hydrogen, deuterium, halogen (e.g., fluorine, chlorine, bromine, or iodine), C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂-etc.), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), C₁₋₆ alkoxy (e.g., C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), or deuterated C₁₋₆ alkoxy;
(Rₐ₅)ₘ indicates that the isoindoline ring to which it is attached is optionally substituted with m Rₐ₈, with each Ras being the same or different and each independently representing halogen (e.g., fluorine, chlorine, bromine, or iodine), hydroxy, mercapto, nitro, amino, cyano, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), C₁₋₆ alkoxy (e.g., C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), deuterated C₁₋₆ alkoxy, C₂₋₆ alkenyl or C₂₋₆ alkynyl;
m represents an integer of 0, 1, 2, 3, or 4;
R_{w} represents O, S, S(O), S(O)₂, alkenylene (e.g., C₂₋₆ alkenylene), alkynylene (e.g., C₂₋₆ alkynylene), or N(Rₐ₆), wherein Rₐ₆ represents H or C₁₋₃ alkyl, or
R_{w} represents a bond, or
R_{w} represents:

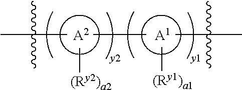

wherein each ring A¹ is the same or different and independently represents nitrogen-containing heterocyclylene (e.g., 4-to 20-membered nitrogen-containing heterocyclylene), arylene (e.g., C₅₋₂₀ arylene), or heteroarylene (e.g., 5-to 20-membered heteroarylene), y1 represents an integer of 1 or 2; and (R_{y1})ₐ₁ indicates that each ring A¹ is independently optionally substituted with a1 R^{y1} substituents, with each R^{y1} independently representing deuterium, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), C₁₋₆ alkoxy (e.g., C₁₋₄ alkoxy, such as methoxy, ethoxy, propoxy, isopropoxy, n-butoxy, sec-butoxy, or tert-butoxy), halogen (e.g., fluorine, chlorine, bromine, or iodine), amino, hydroxy, mercapto, cyano, carboxyl, C₂₋₆ alkynyl, or C₂₋₆ alkenyl, and a1 represents an integer of 0-20;
each ring A² is the same or different and independently represents heterocyclylene (e.g., 4-to 20-membered heterocyclylene), cycloalkylene (e.g., C₃₋₂₀cycloalkylene), arylene (e.g., C₅₋₂₀ arylene), or heteroarylene (e.g., 5-to 20-membered heteroarylene), y2 represents an integer of 0 or 1; and (R^{y2})ₐ2 indicates that each ring A² is independently optionally substituted with a2 R^{y2} substituents, with each R^{y2} independently representing deuterium, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), C₁₋₆ alkoxy (e.g., C₁₋₄ alkoxy, such as methoxy, ethoxy, propoxy, isopropoxy, n-butoxy, sec-butoxy, or tert-butoxy), halogen (e.g., fluorine, chlorine, bromine, or iodine), amino, hydroxy, mercapto, cyano, carboxyl, C₂₋₆ alkynyl, or C₂₋₆ alkenyl, and a2 represents an integer of 0-20; and
R_{w1} represents a bond or optionally substituted linear or branched C₁₋₂₀ alkylene, wherein any one or more (e.g., 1-20, 1-16, 1-14, 1-12, 1-8, 1-6, 1-4, or 2)methylene groups in the main carbon chain skeleton of the linear or branched C₁₋₂₀ alkylene are optionally replaced by one or more (e.g., 1-20, 1-16, 1-14, 1-12, 1-8, 1-6, 1-4, or 2) groups selected from: Rₐ, R_{b}, and any combination thereof; wherein each Rₐ is independently selected from the group consisting of: O, C(O), OC(O), S, S(O), S(O)₂, S(O)₂N(Rₐ₇), N(Rₐ₇) S(O)₂, C(O)N(Rₐ₇), N(Rₐ₇)C(O), N(Rₐ₇), and N(Rₐ₇)C(O)N(Rₐ₇), wherein each Rₐ₇ each independently represents H or C₁₋₃ alkyl, and when any two or more methylene in the main carbon chain skeleton of the linear or branched C₁₋₂₀ alkylene are optionally replaced by two or more Rₐ groups, the Rₐ groups are not directly connected to each other; and wherein each R_{b} groups is independently selected from the group consisting of optionally substituted cycloalkylene (e.g., optionally substituted C₃₋₂₀ cycloalkylene), optionally substituted arylene (e.g., optionally substituted C₅₋₂₀ arylene), optionally substituted heterocyclylene (e.g., optionally substituted 4-to 20-membered heterocyclylene), optionally substituted heteroarylene (e.g., optionally substituted 5-to 20-membered heteroarylene), alkynylene (e.g., C₂₋₆ alkynylene), alkenylene (e.g., C₂₋₆ alkenylene), and any combination thereof, and
R_{w3} represents hydrogen, deuterium, leaving group, hydroxy, halogen, COOH, NH₂, N₃, CHO, methanesulfonyloxy, trifluoromethanesulfonyloxy or p-toluenesulfonyloxy.

The compound of Formula (II) of the present disclosure, or salts (including pharmaceutically acceptable salts), stereoisomers (including enantiomers), solvates, isotopically enriched analogs, prodrugs, or polymorphs thereof, can act as molecular glues binding to the CRBN E3 ubiquitin ligase. The compound of Formula (II) of the present disclosure contains a dihydropyrimidine-2,4-dione-substituted isoindolinone backbone structure (i.e.,

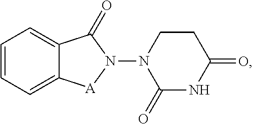

wherein A represents CO or CH₂). The design of this backbone structure skillfully avoids the generation of chirality, which can address the chirality issues associated with existing piperidine-2,6-dione-substituted isoindolinone structures (i.e.,

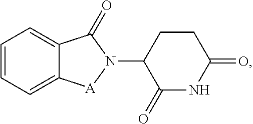

wherein A represents CO or CH₂). It also exhibits CRBN binding affinity, favorable pharmacokinetic properties, or antitumor activity, and can further be applied to bifunctional protein-degrading small-molecule drugs.

In some embodiments, A of the compound of Formula (II) represents CO. In some embodiments, A of the compound of Formula (II) represents CH₂. In some embodiments, A of the compound of Formula (II) represents CD₂.

In some embodiments, Rₐ₁, Rₐ₂, Rₐ₃, and Rₐ₄ of the compound of Formula (II) are the same or different and independently represent hydrogen, deuterium, halogen (e.g., fluorine, chlorine, bromine, or iodine), C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), C₁₋₆ alkoxy (e.g., C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, C₁₂CH—O—, CF₃CF₂—O—, CF₃CHF—O—, CHF₂CF₂—O—, CHF₂CHF—O—, CF₃CH₂—O—, or CH₂ClCH₂—O—), or deuterated C₁₋₆ alkoxy.

In some embodiments, Rₐ₁, Rₐ₂, Rₐ₃, and Rₐ₄ of the compound of Formula (II) represent hydrogen.

In some embodiments, m of the compound of Formula (II) represents an integer of 0, 1, 2, 3, or 4.

In some embodiments, R_{w3} of the compound of Formula (II) represents hydrogen, deuterium, COOH, NH₂, N₃, CHO, halogen, hydroxy, or a leaving group (e.g., —N₃, chlorine, bromine, iodine sulfonate, such as methanesulfonyloxy (MsO—), trifluoromethanesulfonyloxy (TfO—), or p-toluenesulfonyloxy (TsO—)).

In some embodiments, R_{w} of the compound of Formula (II) represents a bond.

In some embodiments, R_{w} of the compound of Formula (II) represents O, S, S(O), S(O)₂, alkenylene (e.g., C₂₋₆alkenylene), alkynylene (e.g., C₂₋₆alkynylene), or N(Rₐ₆), wherein Rₐ₆ represents H or C₁₋₃ alkyl.

In some embodiments, R_{w} of the compound of Formula (II) represents:

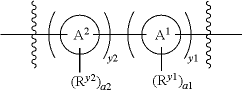

wherein each ring A¹ is the same or different and independently represents 4-to 20-membered (e.g., 4-to 18-membered, 4-to 15-membered, 4-to 12-membered, 4-to 11-membered, 4-to 10-membered, 4-to 9-membered, 4-to 8-membered, 4-to 7-membered, 4-to 6-membered, 5-to 15-membered, or 5-to 9-membered) nitrogen-containing heterocyclylene, C₅₋₂₀ arylene (e.g., C₆₋₂₀ arylene, C₅₋₁₅ arylene, or C₆₋₁₅ arylene), or 5-to 20-membered heteroarylene (e.g., 5-to 15-membered, 5-to 10-membered, 5-to 9-membered, 5-to 8-membered, 5-to 7-membered, or 5-to 6-membered heteroarylene). In some embodiments, 4-to 20-membered nitrogen-containing heterocyclylene is a 4-to 20-membered (e.g., 4-to 15-membered, 4-to 12-membered, 4-to 11-membered, 4-to 10-membered, 4-to 9-membered, 4-to 8-membered, 4-to 7-membered, 4-to 6-membered, 5-to 15-membered, or 5-to 9-membered) heterocyclylene containing one nitrogen atom and optionally containing a heteroatom(s) selected from nitrogen, oxygen, and sulfur. Examples of the 4-to 20-membered heterocyclylene include, but are not limited to, e.g., piperidinylene, piperazinylene, morpholinylene, azetidinylene, pyrrolidinylene, imidazolidylene, pyrazolidylene, oxazolidinylene, thiazolidinylene, thiomorpholinylene, azepanylene, diazacycloheptanylene, azacyclooctylene, diazacyclooctylene, azabicyclo[3.1.1]heptanylene, azabicyclo[2.2.1]heptanylene, azabicyclo[3.2.1]octanylene, azabicyclo[2.2.2]octanylene, diazabicyclo[3.1.1]heptanylene, diazabicyclo[2.2.1]heptanylene, diazabicyclo[3.2.1]octanylene, diazabicyclo[2.2.2]octanylene, 3-azaspiro[5.5]undecanylene, 8-azaspiro[4.5]decanylene, 2-oxa-8-azaspiro[4.5]decanylene, or 3-methyl-2-oxa-8-azaspiro[4.5]decanylene. In some embodiments, examples of the 5-to 20-membered arylene include, but are not limited to, e.g., phenylene or naphthylene. In some embodiments, the 5-to 20-membered heteroarylene is a 5-to 20-membered (e.g., 5-to 15-membered, 5-to 12-membered, 5-to 9-membered, 5-to 8-membered, 5-to 7-membered, or 5-to 6-membered) heteroarylene containing one nitrogen atom and optionally containing a heteroatom(s) selected from nitrogen, oxygen, and sulfur. In some embodiments, examples of the 5-to 20-membered heteroarylene include, but are not limited to, e.g., oxazolylene, isoxazolylene, oxadiazolylene, thienylene, thiazolylene, isothiazolylene, thiadiazolylene, pyrrolylene, imidazolylene, pyrazolylene, triazolylene, pyridylene, pyrimidinylene, pyridazinylene, pyrazinylene, indolylene, isoindolylene, benzofuranylene, isobenzofuranylene, benzothienylene, indazolylene, benzimidazolylene, benzoxazolylene, benzisoxazolylene, benzothiazolylene, benzisothiazolylene, benzotriazolylene, benzo[2,1,3]oxadiazolylene, benzo[2,1,3]thiadiazolylene, benzo[1,2,3]thiadiazolylene, quinolinylene, isoquinolinylene, naphthyridinylene, cinnolinylene, quinazolinylene, quinoxalinylene, phthalazinylene, pyrazolo[1,5-a]pyridylene, pyrazolo[1,5-a]pyrimidinylene, imidazo[1,2-a]pyridylene, 1H-pyrrolo[3,2-b]pyridylene, 1H-pyrrolo[2,3-b]pyridylene, pyrrolo[2,1-b]thiazolylene, and imidazo[2,1-b]thiazolylene. Each ring A¹ is independently optionally substituted with a1 R^{y1} substituents, and each R_{y1} independently represents deuterium, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), C₁₋₆ alkoxy (e.g., C₁₋₄ alkoxy, such as methoxy, ethoxy, propoxy, isopropoxy, n-butoxy, sec-butoxy, or tert-butoxy), halogen (e.g., fluorine, chlorine, bromine, or iodine), amino, hydroxy, mercapto, cyano, carboxyl, C₂₋₆ alkynyl, or C₂₋₆ alkenyl. a1 represents an integer of 0-20, e.g., an integer of 0, 1, 2, 3, 4, 5, 6, 7, 8, 9, 10, 11, 12, 13, 14, 15, or 20. The number of substituents is not theoretically limited in any way, or is automatically limited by the size of the building units.

In some embodiments, y1 of the compound of Formula (II) represents an integer of 1.

In some embodiments, y1 of the compound of Formula (II) represents an integer of 2.

In some embodiments, each ring A¹ of the compound of Formula (II) is the same or different and independently represents the following optionally substituted groups:

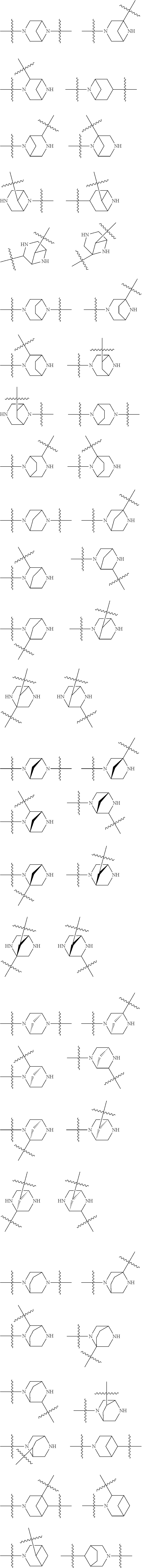

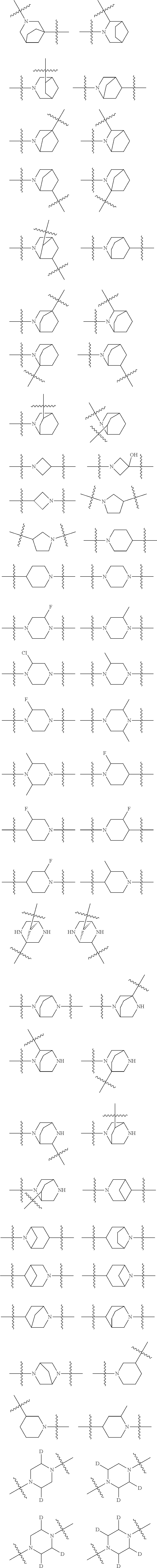

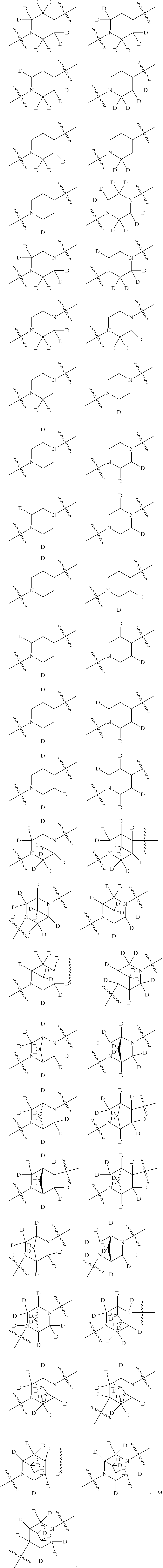

each ring A¹ is independently optionally substituted with a1 R_{y1} substituents, and each R^{y1} independently represents deuterium, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), C₁₋₆ alkoxy (e.g., C₁₋₄ alkoxy, such as methoxy, ethoxy, propoxy, isopropoxy, n-butoxy, sec-butoxy, or tert-butoxy), halogen (e.g., fluorine, chlorine, bromine, or iodine), amino, hydroxy, mercapto, cyano, carboxyl, C₂₋₆ alkynyl (e.g., C₂₋₄ alkynyl, such as ethynyl, propynyl or butynyl), or C₂₋₆ alkenyl (e.g., C₂₋₄ alkenyl, such as vinyl, propenyl, or butenyl). a1 represents an integer of 0-20, e.g., an integer of 0, 1, 2, 3, 4, 5, 6, 7, 8, 9, 10, 11, 12, 13, 14, 15, or 20. The number of substituents is not theoretically limited in any way, or is automatically limited by the size of the building units.

In some embodiments, each ring A² in the compound of Formula (II) is the same or different and independently represents heterocyclylene (e.g., 4-to 20-membered heterocyclylene, such as 4-to 18-membered, 4-to 15-membered, 4-to 12-membered, 4-to 11-membered, 4-to 10-membered, 4-to 9-membered, 4-to 8-membered, 4-to 7-membered, 4-to 6-membered, 5-to 15-membered, or 5-to 9-membered heterocyclylene), cycloalkylene (e.g., C₃₋₂₀cycloalkylene, C₃₋₁₅cycloalkylene, or C₃₋₁₁cycloalkylene), arylene (e.g., C₅₋₂₀ arylene, such as C₆₋₂₀ arylene, C₅₋₁₅ arylene, or C₆₋₁₅ arylene), or heteroarylene (e.g., 5-to 20-membered heteroarylene, such as 5-to 15-membered, 5-to 10-membered, 5-to 9-membered, 5-to 8-membered, 5-to 7-membered, or 5-to 6-membered heteroarylene). In some embodiments, the 4-to 20-membered heterocyclylene is a 4-to 20-membered (e.g., 4-to 15-membered, 4-to 12-membered, 4-to 11-membered, 4-to 10-membered, 4-to 9-membered, 4-to 8-membered, 4-to 7-membered, 4-to 6-membered, 5-to 15-membered, or 5-to 9-membered) saturated or partially unsaturated (i.e., containing one or more double bonds, but not having a fully conjugated π-electron system) divalent monocyclic, bicyclic, tricyclic or polycyclic cyclic hydrocarbon group containing one or more (e.g., from 1 to 5, from 1 to 4, or from 1 to 3, or from 1 to 2, or 1) heteroatoms selected from the group consisting of oxygen, nitrogen, and sulfur. Examples of the 4-to 20-membered heterocyclylene include, but are not limited to, monocyclic heterocyclylene (e.g., 4-to 20-membered monocyclic heterocyclylene), bridged heterocyclylene (e.g., 5-to 20-membered bridged heterocyclylene, or 7-to 15-membered bridged heterocyclylene), fused heterocyclylene, and spiro-heterocyclylene (e.g., 5-to 20-membered spiro-heterocyclylene), e.g., azetidinylene, oxetanylene, pyrrolidinylene, imidazolidinylene, pyrazolidylene, tetrahydrofuranylene, tetrahydropyranylene, tetrahydrothienylene, tetrahydrothiopyranylene, oxazolidinylene, thiazolidinylene, piperidinylene, piperazinylene, morpholinylene, thiomorpholinylene, azacycloheptanylene, azacyclooctylene, dioxacyclohexylene, diazacycloheptanylene, diazacyclooctylene, 6-azabicyclo[3.1.1]heptanylene, 2,5-diazabicyclo[2.2.1]heptanylene, 3,6-diazabicyclo[3.1.1]heptanylene, 3-azabicyclo[3.2.1]octanylene, 3,8-diazabicyclo[3.2.1]octanylene, 3,8-diazabicyclo[3.2.1]octanylene, 2,5-diazabicyclo[2.2.2]octanylene, and azaspirocycloalkylene (e.g., 5-to 20-membered azaspirocycloalkylene, such as 3-azaspiro[5.5]undecanylene). In some embodiments, examples of the C₃₋₂₀ cycloalkylene include, but are not limited to, e.g., cyclopropylene, cyclobutylene, cyclopentylene, cyclopentenylene, cyclohexylene, cyclohexenylene, cycloheptylene, cyclooctylene, decalinylene, octahydropentalenylene, octahydro-1H-indenylene, 2,3-dihydro-1H-indenylene, spiro-cycloalkylene (e.g., C₅₋₁₅ spiro-cycloalkylene), adamantanylene, noradamantanylene, and norbornylene. In some embodiments, examples of the 5-to 20-membered arylene include, but are not limited to, e.g., phenylene or naphthylene. In some embodiments, 5-to 20-membered heteroarylene is a 5-to 20-membered (e.g., 5-to 15-membered, 5-to 12-membered, 5-to 9-membered, 5-to 8-membered, 5-to 7-membered, or 5-to 6-membered) heteroarylene containing one nitrogen atom and optionally containing a heteroatom(s) selected from nitrogen, oxygen, and sulfur. In some embodiments, examples of the 5-to 20-membered heteroarylene include, but are not limited to, e.g., oxazolylene, isoxazolylene, oxadiazolylene, thienylene, thiazolylene, isothiazolylene, thiadiazolylene, pyrrolylene, imidazolylene, pyrazolylene, triazolylene, pyridylene, pyrimidinylene, pyridazinylene, pyrazinylene, indolylene, isoindolylene, benzofuranylene, isobenzofuranylene, benzothienylene, indazolylene, benzimidazolylene, benzoxazolylene, benzisoxazolylene, benzothiazolylene, benzisothiazolylene, benzotriazolylene, benzo[2,1,3]oxadiazolylene, benzo[2,1,3]thiadiazolylene, benzo[1,2,3]thiadiazolylene, quinolinylene, isoquinolinylene, naphthyridinylene, cinnolinylene, quinazolinylene, quinoxalinylene, phthalazinylene, pyrazolo[1,5-a]pyridylene, pyrazolo[1,5-alpyrimidinylene, imidazo[1,2-a]pyridylene, 1H-pyrrolo[3,2-b]pyridylene, 1H-pyrrolo[2,3-b]pyridylene, pyrrolo[2,1-b]thiazolylene, and imidazo[2,1-b]thiazolylene. Each ring A² is independently optionally substituted with a2 R^{y2} substituents, and each R^{y2} independently represents deuterium, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), C₁₋₆ alkoxy (e.g., C₁₋₄ alkoxy, such as methoxy, ethoxy, propoxy, isopropoxy, n-butoxy, sec-butoxy, or tert-butoxy), halogen (e.g., fluorine, chlorine, bromine, or iodine), amino, hydroxy, mercapto, cyano, carboxyl, C₂₋₆ alkynyl, or C₂₋₆ alkenyl. a2 represents an integer of 0-20, e.g., an integer of 0, 1, 2, 3, 4, 5, 6, 7, 8, 9, 10, 11, 12, 13, 14, 15, or 20. The number of substituents is not theoretically limited in any way, or is automatically limited by the size of the building units.

In some embodiments, each ring A² of the compound of Formula (II) is the same or different and independently represents the following optionally substituted groups:

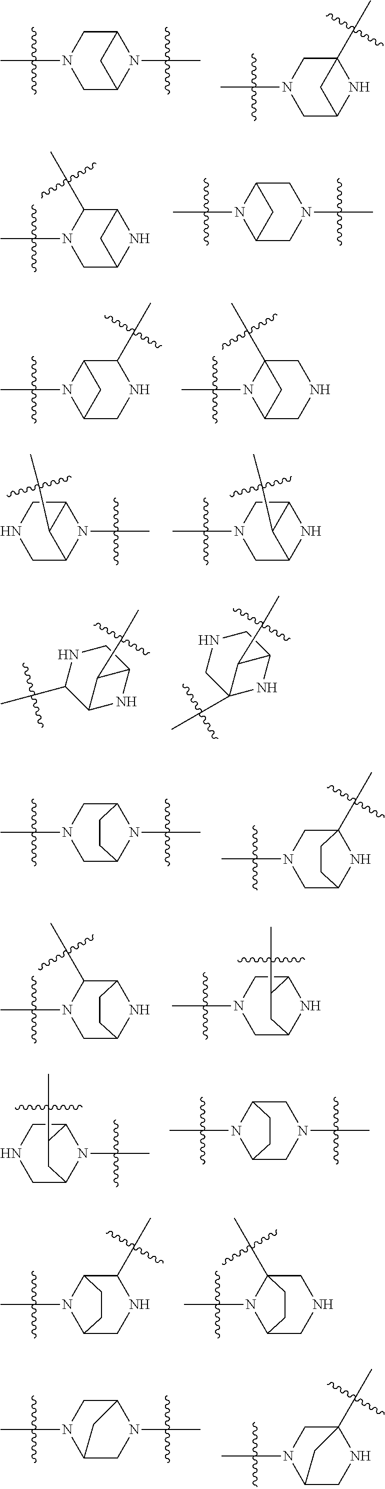

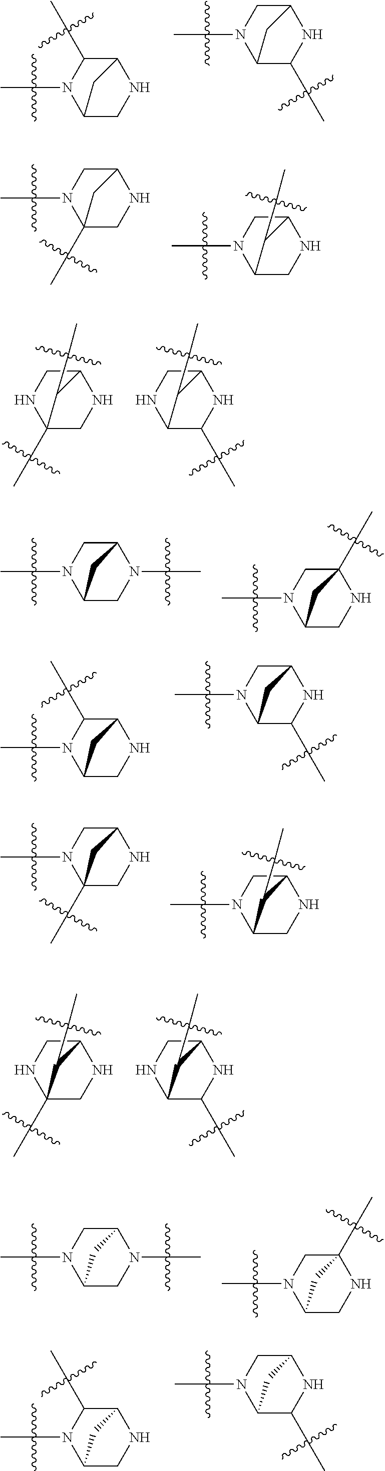

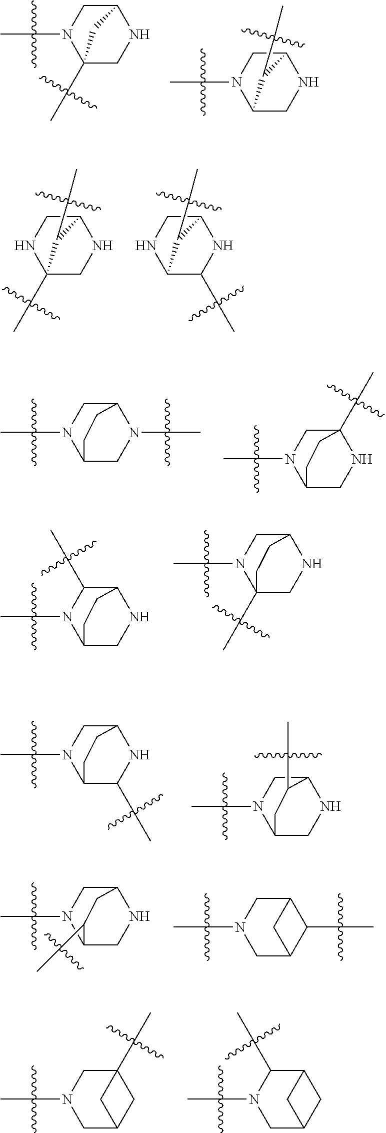

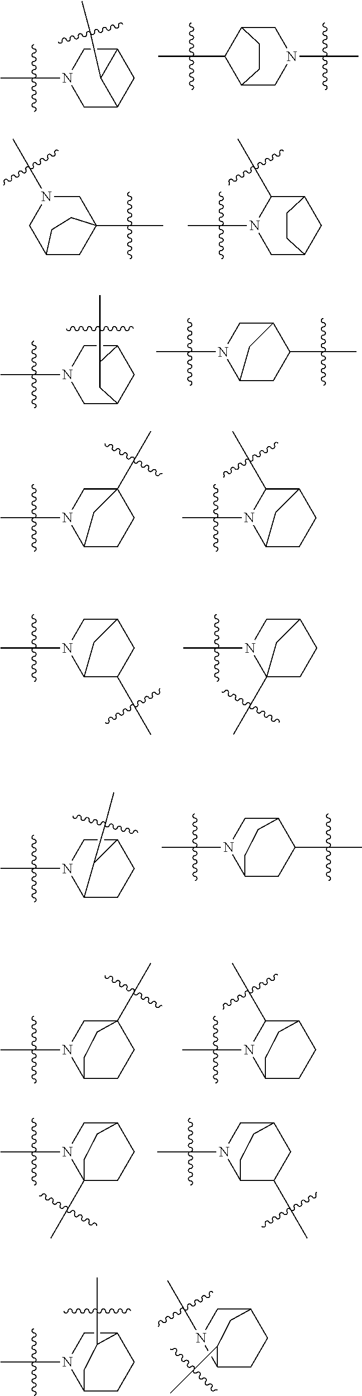

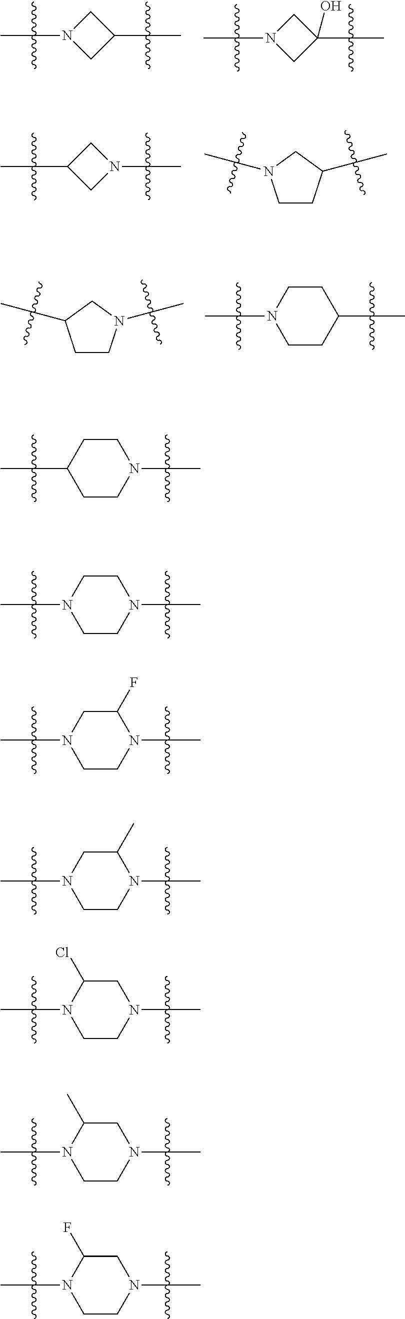

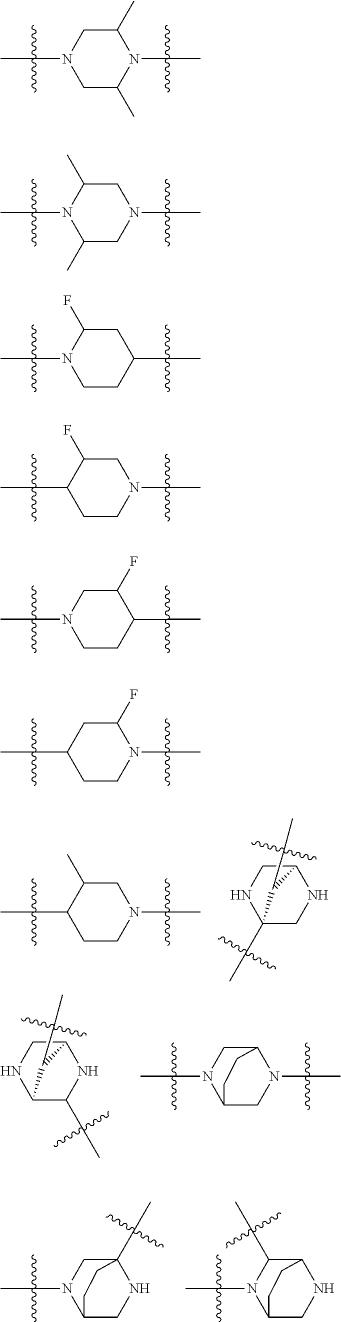

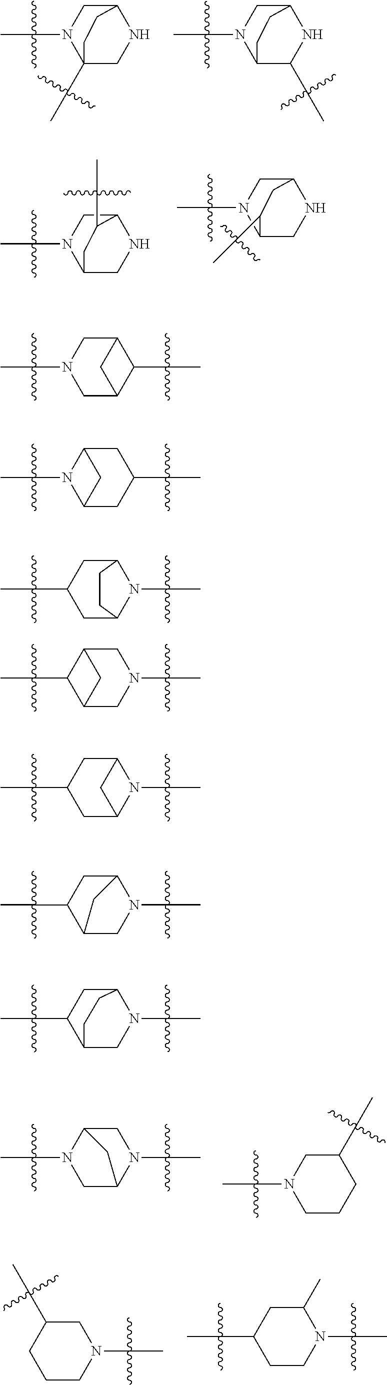

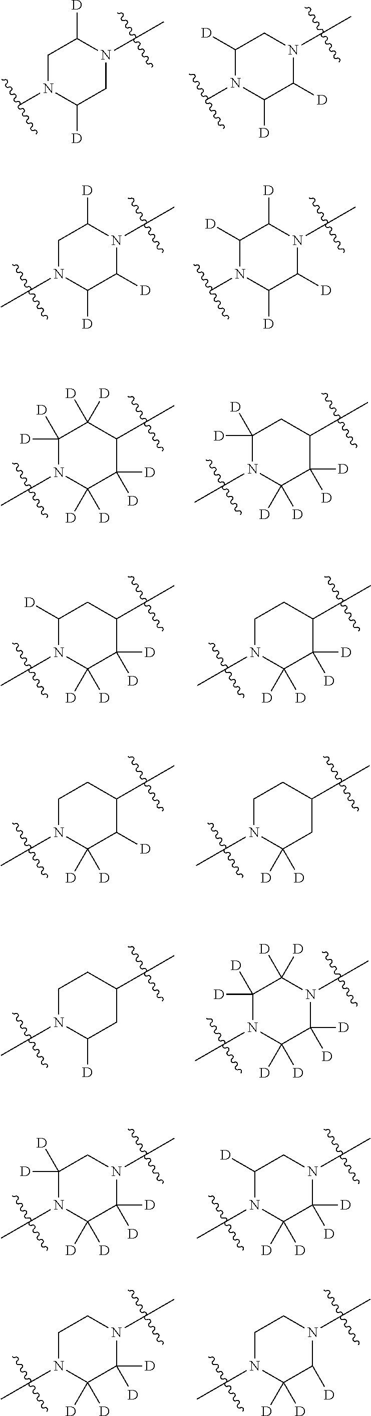

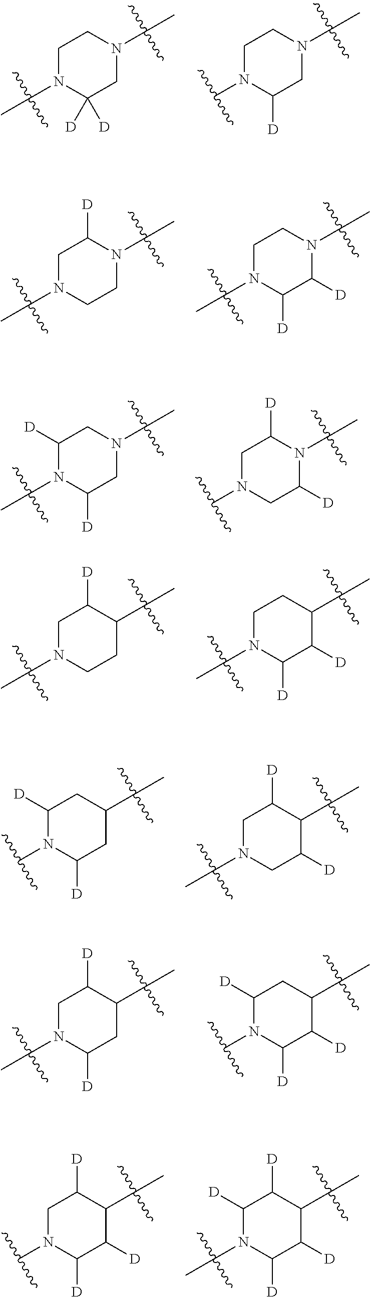

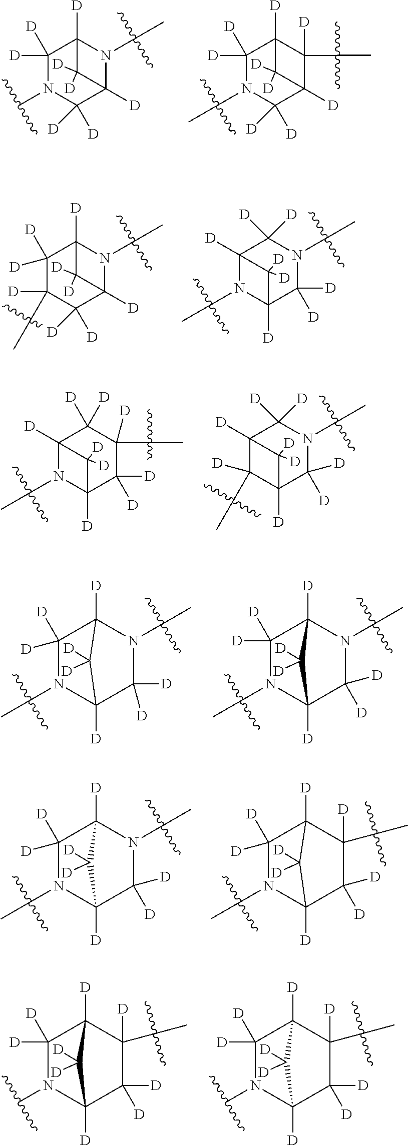

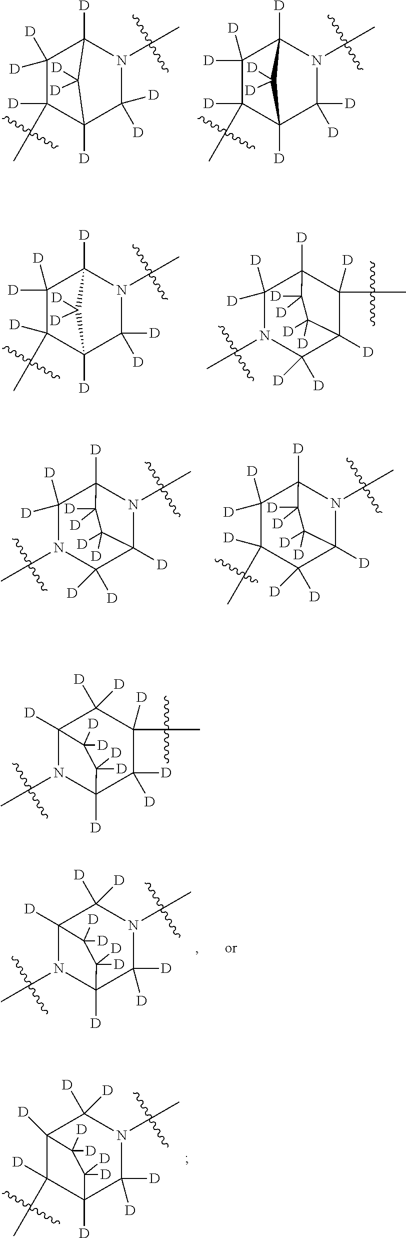

each ring A² is independently optionally substituted with a2 R^{y2} groups, wherein each R^{y2} is independently deuterium, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), C₁₋₆ alkoxy (e.g., C₁₋₄ alkoxy, such as methoxy, ethoxy, propoxy, isopropoxy, n-butoxy, sec-butoxy, or tert-butoxy), halogen (e.g., fluorine, chlorine, bromine, or iodine), amino, hydroxy, mercapto, cyano, carboxyl, C₂₋₆ alkynyl (e.g., C₂₋₄ alkynyl, such as ethynyl, propynyl or butynyl), or C₂₋₆ alkenyl (e.g., C₂₋₄ alkenyl, such as vinyl, propenyl, or butenyl). a2 represents an integer of 0-20, e.g., an integer of 0, 1, 2, 3, 4, 5, 6, 7, 8, 9, 10, 11, 12, 13, 14, 15, or 20. The number of substituents is not theoretically limited in any way, or is automatically limited by the size of the building units.

In some embodiments, y2 of the compound of Formula (II) represents an integer of 0.

In some embodiments, y2 of the compound of Formula (II) represents an integer of 1.

In some embodiments, R_{w} of the compound of Formula (II) represents:

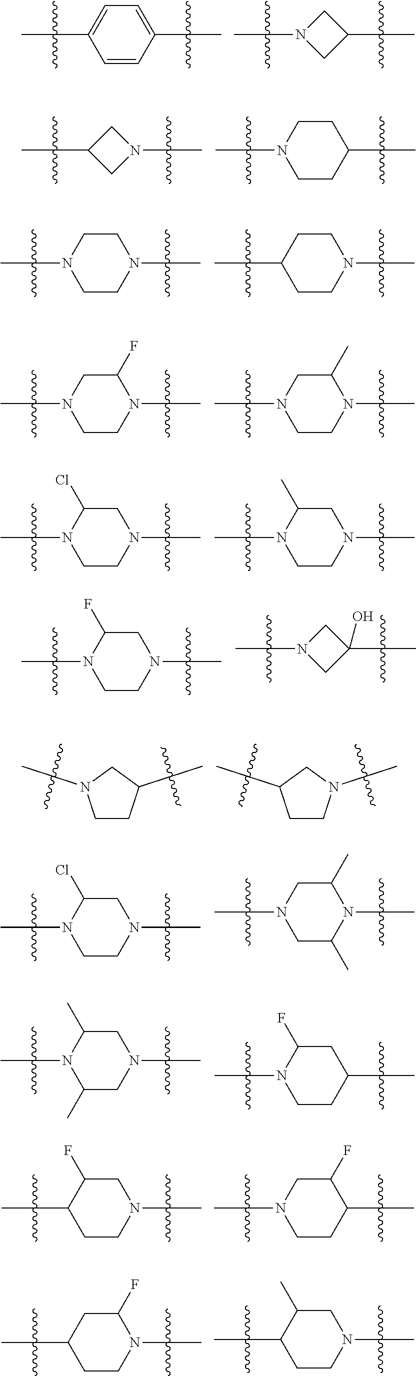

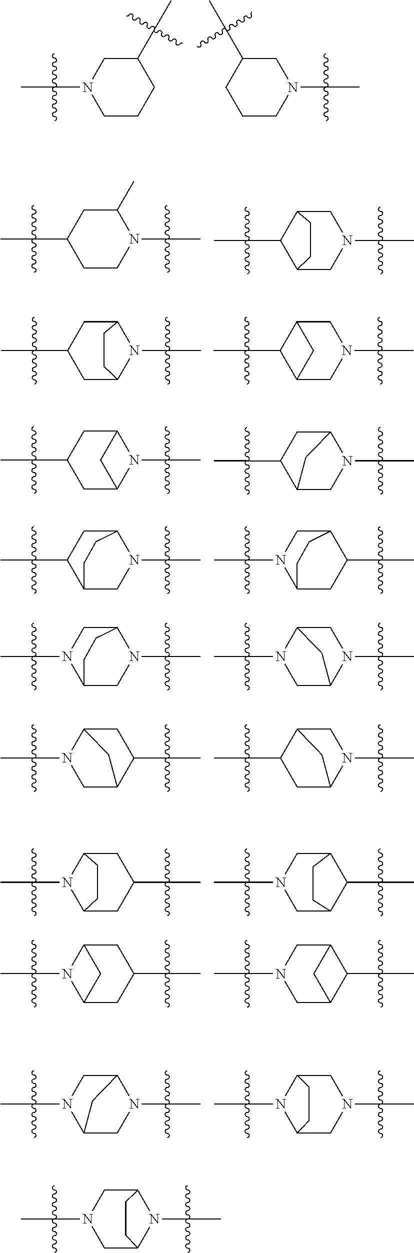

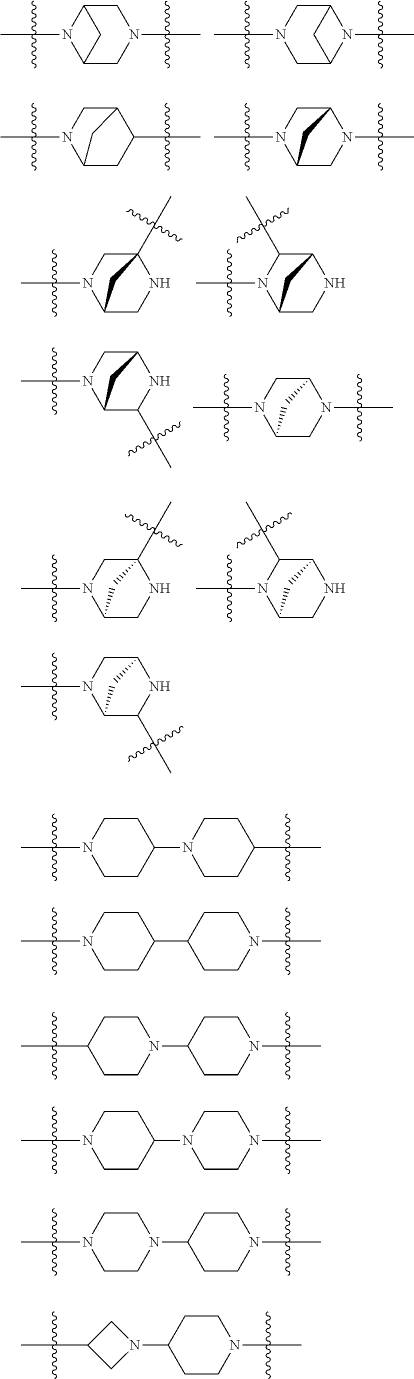

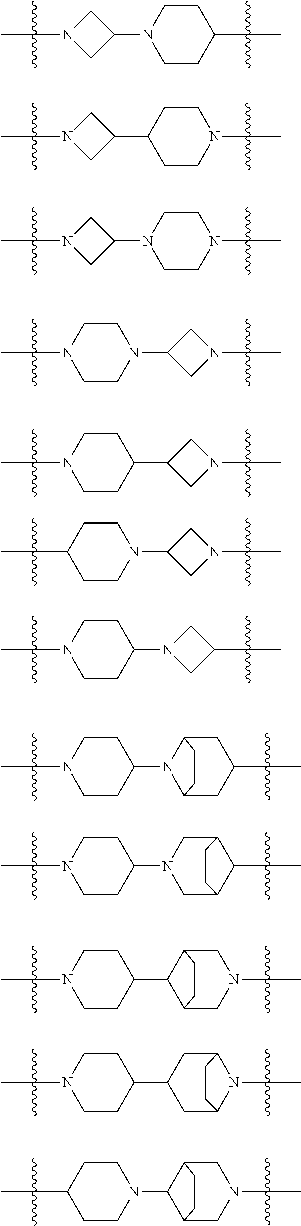

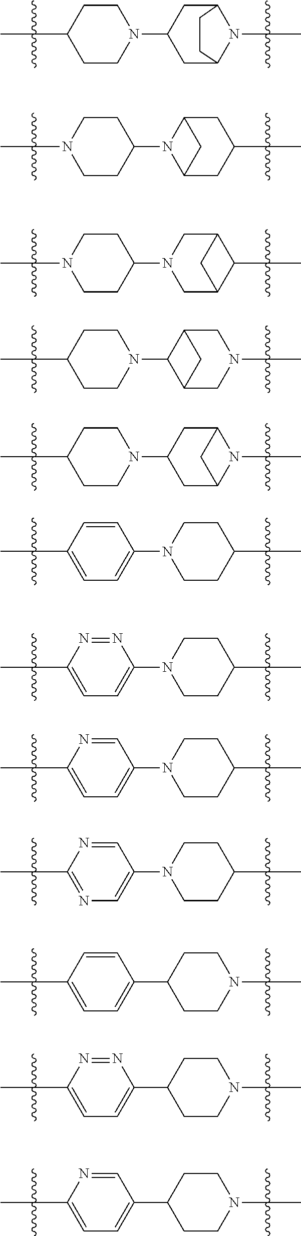

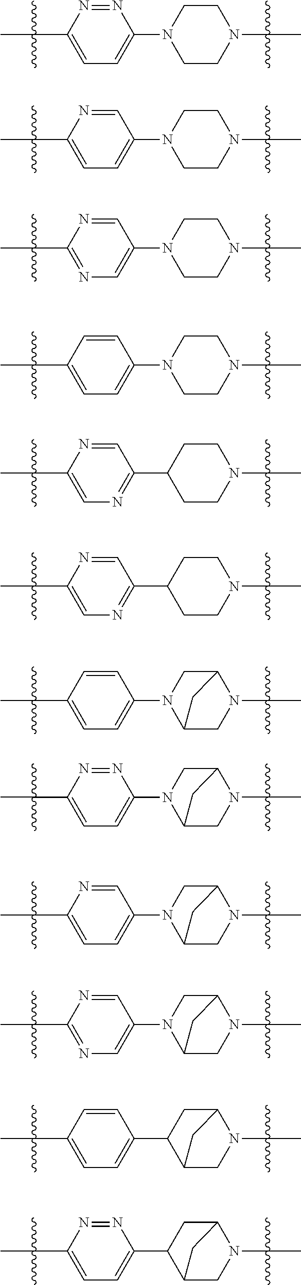

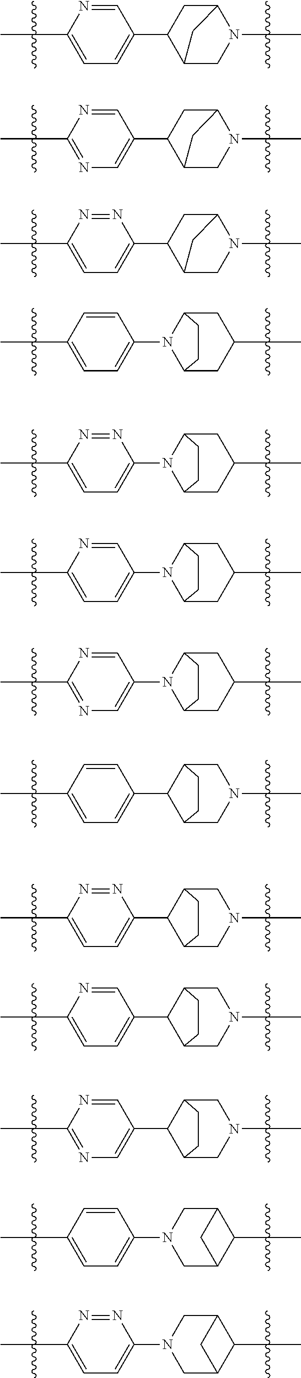

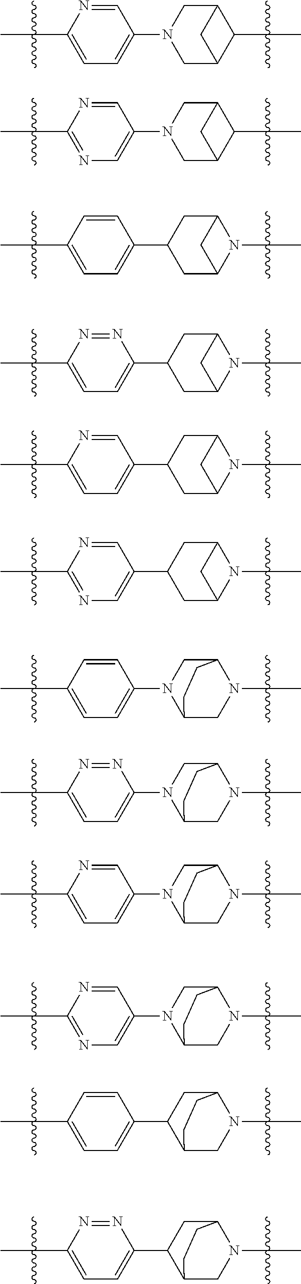

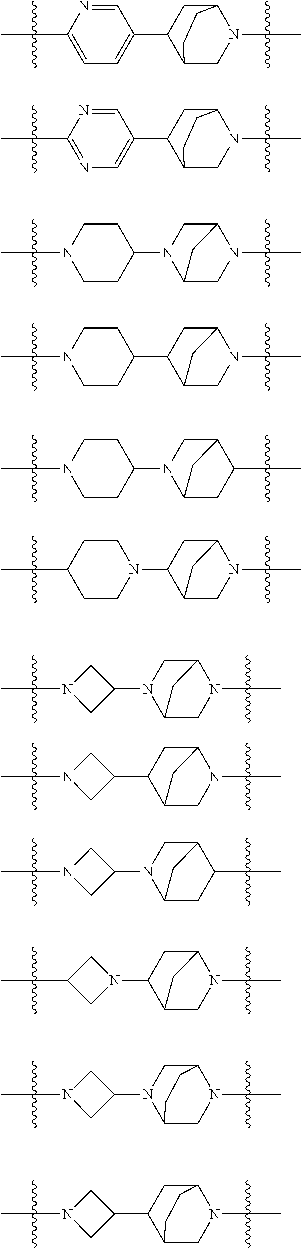

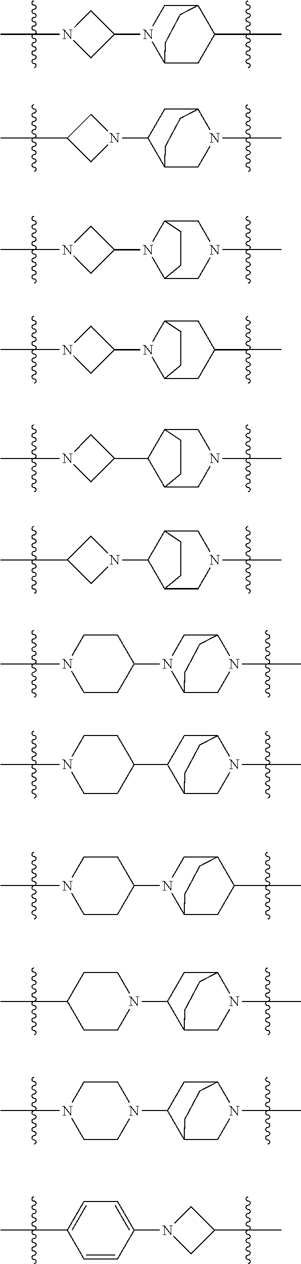

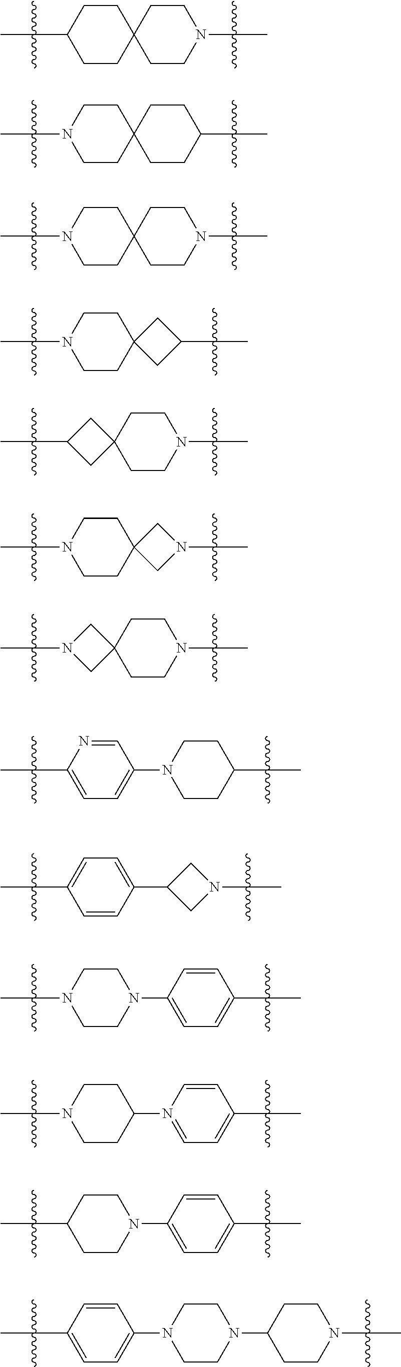

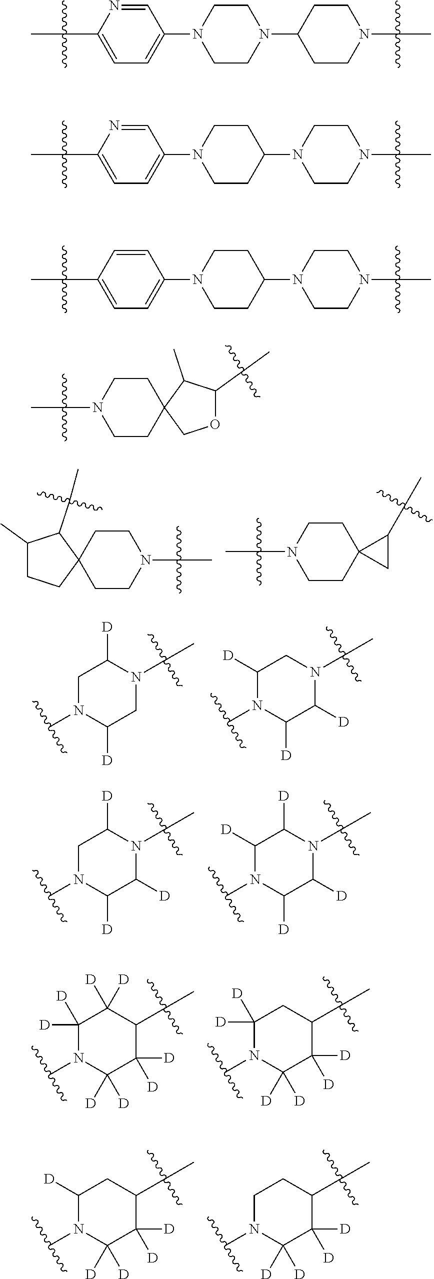

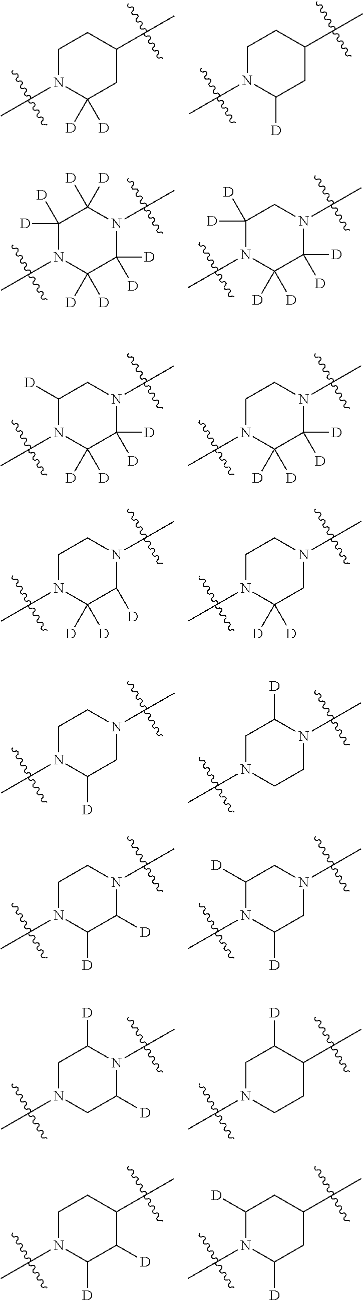

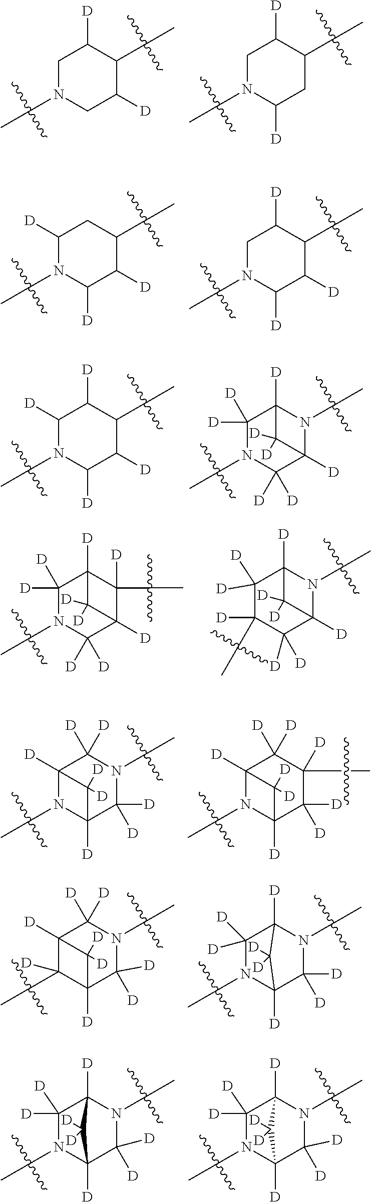

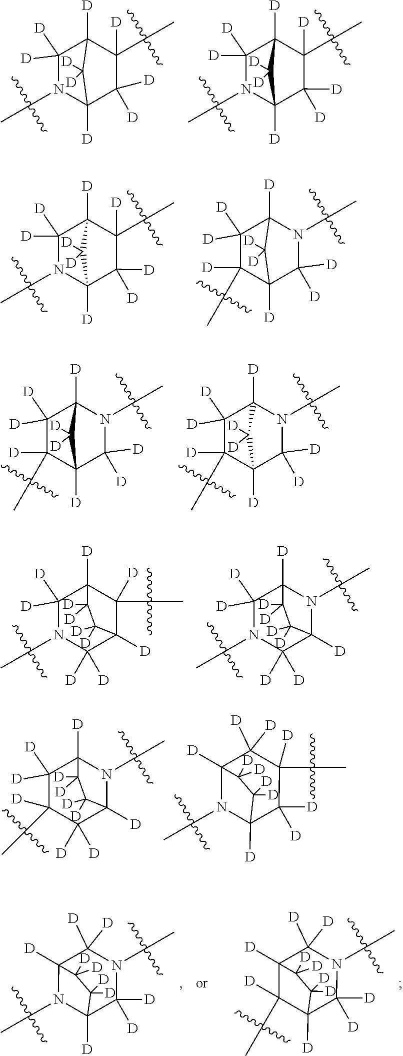

wherein the groups are optionally substituted with a substituent selected from the group consisting of deuterium, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), C₁₋₆ alkoxy (e.g., C₁₋₄ alkoxy, such as methoxy, ethoxy, propoxy, isopropoxy, n-butoxy, sec-butoxy, or tert-butoxy), halogen (e.g., fluorine, chlorine, bromine, or iodine), amino, hydroxy, mercapto, cyano, carboxyl, C₂₋₆ alkynyl (e.g., C₂₋₄ alkynyl, such as ethynyl, propynyl or butynyl), or C₂₋₆ alkenyl (e.g., C₂₋₄ alkenyl, such as vinyl, propenyl, or butenyl).

In some embodiments, R_{w1} of the compound of Formula (II) represents a bond.

In some embodiments, R_{w1} of the compound of Formula (II) represents optionally substituted linear or branched C₁₋₂₀ alkylene, wherein any one or more (e.g., 1-15, 1-10, 1-9, 1-8, 1-7, 1-6, 1-5, 1-4, 1-3, or 1-2, or 1) methylene groups in the main carbon chain skeleton of the linear or branched C₁₋₂₀ alkylene are optionally replaced by one or more (e.g., 1-20 or 1-15, 1-10, 1-9, 1-8, 1-7, 1-6, 1-5, 1-4, 1-3, or 1-2, or 1) groups selected from: Rₐ, R_{b}, and any combination thereof; wherein each Rₐ is independently selected from the group consisting of: O, C(O), OC(O), S, S(O), S(O)₂, S(O)₂N(Rₐ₇), N(Rₐ₇) S(O)₂, C(O)N(Rₐ₇), N(Rₐ₇)C(O), N(Rₐ₇), and N(Rₐ₇)C(O)N(Rₐ₇), wherein each Raz independently represents H or C₁₋₃ alkyl, and when any two or more methylene in the main carbon chain skeleton of the linear or branched C₁₋₂₀ alkylene are optionally replaced by two or more Rₐ groups, the Rₐ groups are not directly connected to each other; and wherein each R_{b} is independently selected from the group consisting of: optionally substituted cycloalkylene (e.g., optionally substituted C₃₋₂₀ cycloalkylene), optionally substituted arylene (e.g., optionally substituted C₅₋₂₀ arylene), optionally substituted heterocyclylene (e.g., optionally substituted 4-to 20-membered heterocyclylene), optionally substituted heteroarylene (e.g., optionally substituted 5-to 20-membered heteroarylene), alkynylene (e.g., C₂₋₆alkynylene), alkenylene (e.g., C₂₋₆ alkenylene), and any combination thereof, wherein the cycloalkylene, arylene, heterocyclylene, and heteroarylene are each independently optionally substituted with a substituent selected from the group consisting of deuterium, hydroxy, amino, mercapto, nitro, halogen, cyano, oxo, C₁₋₆ alkyl (e.g., C₁₋₅ alkyl, C₁₋₄ alkyl, or C₁₋₃ alkyl, such as methyl, ethyl, propyl, isopropyl, butyl, sec-butyl, tert-butyl, pentyl, or hexyl), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), C₁₋₆ alkoxy (e.g., C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), C₃₋₆ cycloalkyl, halogenated C₃₋₆ cycloalkyl, deuterated C₃₋₆ cycloalkyl, 4-to 10-membered heterocyclyl, and any combination thereof; and one or more hydrogens of said linear or branched C₁₋₂₀ alkylene are optionally replaced by a substituent selected from: deuterium, hydroxy, amino, mercapto, nitro, halogen, cyano, deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), C₁₋₆ alkoxy (e.g., C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), C₃₋₆ cycloalkyl, halogenated C₃₋₆ cycloalkyl, deuterated C₃₋₆ cycloalkyl, 4-to 10-membered heterocyclyl, and any combination thereof.

In some embodiments, R_{w1} of the compound of Formula (II) represents a structure of the following Formulae:
—C₁₋₁₅ alkylene-;
—(C(Rₐ₈)(Rₐ₉))ₙ₁—(Rₐ(C(Rₐ₁₀)(Rₐ₁₁))ₙ₂)ₘ₃—;
—(C(Rₐ₈)(Rₐ₉))ₙ₁—(Rₐ(C(Rₐ₁₀)(Rₐ₁₁))ₙ₂)ₘ₃—(Rₐ(C(Rₐ₁₂)(Rₐ₁₃))ₙ₃)ₘ₄—;
—(C(Rₐ₈)(Rₐ₉))ₙ₁—(Rₐ(C(Rₐ₁₀)(Rₐ₁₁))ₙ₂)ₘ₃—(Rₐ(C(Rₐ₁₂)(Rₐ₁₃))ₙ₃)ₘ₄—(Rₐ(C(Rₐ₁₄)(Rₐ₁₅))ₙ₄)ₘ₅—;
—(C(Rₐ₈)(Rₐ₉))ₙ₁—((C(Rₐ₁₀)(Rₐ₁₁))ₙ₂Rₐ)ₘ₃—;
—(C(Rₐ₈)(Rₐ₉))ₙ₁—((C(Rₐ₁₀)(Rₐ₁₁))ₙ₂Rₐ)ₘ₃—((C(Rₐ₁₂)(Rₐ₁₃))ₙ₃Rₐ)ₘ₄—;
—(C(Rₐ₈)(Rₐ₉))ₙ₁—((C(Rₐ₁₀)(Rₐ₁₁))ₙ₂Rₐ)ₘ₃—((C(Rₐ₁₂)(Rₐ₁₃))ₙ₃Rₐ)ₘ₄—((C(Rₐ₁₄)(Rₐ₁₅))ₙ₄Rₐ)ₘ₅—;
—(C(Rₐ₈)(Rₐ₉))ₙ₁—(R_{b}(C(Rₐ₁₀)(Rₐ₁₁))ₙ₂)ₘ₃—;
—(C(Rₐ₈)(Rₐ₉))ₙ₁—(R_{b}(C(Rₐ₁₀)(Rₐ₁₁))ₙ₂)ₘ₃—(R_{b}(C(Rₐ₁₂)(Rₐ₁₃))ₙ₃)ₘ₄—;
—(C(Rₐ₈)(Rₐ₉))ₙ₁—(R_{b}(C(Rₐ₁₀)(Rₐ₁₁))ₙ₂)ₘ₃—(R_{b}(C(Rₐ₁₂)(Rₐ₁₃))ₙ₃)ₘ₄—(R_{b}(C(Rₐ₁₄)(Rₐ₁₅))ₙ₄)ₘ₅—;
—(C(Rₐ₈)(Rₐ₉))ₙ₁—((C(Rₐ₁₀)(Rₐ₁₁))ₙ₂R_{b})ₘ₃—;
—(C(Rₐ₈)(Rₐ₉))ₙ₁—((C(Rₐ₁₀)(Rₐ₁₁))ₙ₂R_{b})ₘ₃—((C(Rₐ₁₂)(Rₐ₁₃))ₙ₃R_{b})ₘ₄—;
—(C(Rₐ₈)(Rₐ₉))ₙ₁—((C(Rₐ₁₀)(Rₐ₁₁))ₙ₂R_{b})ₘ₃—((C(Rₐ₁₂)(Rₐ₁₃))ₙ₃R_{b})ₘ₄—((C(Rₐ₁₄)(Rₐ₁₅))ₙ₄R_{b})ₘ₅—;
—(C(Rₐ₈)(Rₐ₉))ₙ₁—(Rₐ—R_{b}—(C(Rₐ₁₀)(Rₐ₁₁))ₙ₂)ₘ₃—;
—(C(Rₐ₈)(Rₐ₉))ₙ₁—(Rₐ(C(Rₐ₁₀)(Rₐ₁₁))ₙ₂)ₘ₃—(R_{b}(C(Rₐ₁₂)(Rₐ₁₃))ₙ₃)ₘ₄—;
—(C(Rₐ₈)(Rₐ₉))ₙ₁—(R_{b}—Rₐ—(C(Rₐ₁₀)(Rₐ₁₁))ₙ₂)ₘ₃—;
—(C(Rₐ₈)(Rₐ₉))ₙ₁—(R_{b}(C(Rₐ₁₀)(Rₐ₁₁))ₙ₂)ₘ₃—(Rₐ(C(Rₐ₁₂)(Rₐ₁₃))ₙ₃)ₘ₄—;
—(C(Rₐ₈)(Rₐ₉))ₙ₁—((C(Rₐ₁₀)(Rₐ₁₁))ₙ₂—Rₐ—R_{b})ₘ₃—;
—(C(Rₐ₈)(Rₐ₉))ₙ₁—((C(Rₐ₁₀)(Rₐ₁₁))ₙ₂Rₐ)ₘ₃—((C(Rₐ₁₂)(Rₐ₁₃))ₙ₃R_{b})ₘ₄—;
—(C(Rₐ₈)(Rₐ₉))ₙ₁—((C(Rₐ₁₀)(Rₐ₁₁))ₙ₂—R_{b}—Rₐ)ₘ₃—, or
—(C(Rₐ₈)(Rₐ₉))ₙ₁—((C(Rₐ₁₀)(Rₐ₁₁))ₙ₂R_{b})ₘ₃—((C(Rₐ₁₂)(Rₐ₁₃))ₙ₃Rₐ)ₘ₄—;
wherein each Rₐ is independently selected from the group consisting of O, C(O), OC(O), S, S(O), S(O)₂, S(O)₂N(Rₐ₇), N(Rₐ₇) S(O)₂, C(O)N(Rₐ₇), N(Rₐ₇)C(O), N(Rₐ₇), and N(Rₐ₇)C(O)N(Rₐ₇), wherein each Rₐ₇ independently represents H or C₁₋₃ alkyl; and each R_{b} is independently selected from the group consisting of optionally substituted cycloalkylene (e.g., optionally substituted C₃-20cycloalkylene), optionally substituted arylene (e.g., optionally substituted C₅₋₂₀ arylene), optionally substituted heterocyclylene (e.g., optionally substituted 4-to 20-membered heterocyclylene), optionally substituted heteroarylene (e.g., 5-to 20-membered heteroarylene), alkynylene (e.g., C₂₋₆alkynylene), alkenylene (e.g., C₂₋₆alkenylene), and any combination thereof, wherein the cycloalkylene, arylene, heterocyclylene, and heteroarylene are each independently optionally substituted with a substituent selected from the group consisting of deuterium, hydroxy, amino, mercapto, nitro, halogen, cyano, oxo, C₁₋₆ alkyl, halogenated C₁₋₆ alkyl, deuterated C₁₋₆ alkyl, C₁₋₆ alkoxy, halogenated C₁₋₆ alkoxy, C₃₋₆ cycloalkyl, halogenated C₃₋₆ cycloalkyl, deuterated C₃₋₆ cycloalkyl, 4-to 10-membered heterocyclyl, and any combination thereof;
Rₐ₈, Rₐ₉, Rₐ₁₀, Rₐ₁₁, Rₐ₁₂, Rₐ₁₃, Rₐ₁₄, and Rₐ₁₅ each independently represent H, deuterium, hydroxy, amino, mercapto, nitro, halogen, cyano, oxo, halogenated C₁₋₆ alkyl, deuterated C₁₋₆ alkyl, C₁₋₆ alkoxy, halogenated C₁₋₆ alkoxy, C₃₋₆ cycloalkyl, halogenated C₃₋₆ cycloalkyl, deuterated C₃₋₆ cycloalkyl, 4-to 10-membered heterocyclyl, and any combination thereof, and
n1, n2, n3, n4, m3, m4, and m5 each independently represent an integer of 1, 2, 3, 4, 5, 6, 7, 8, 9, or 10; and
any one or more hydrogens of said C₁₋₁₅ alkylene are optionally replaced by a substituent selected from: deuterium, hydroxy, amino, mercapto, nitro, halogen, cyano, halogenated C₁₋₆ alkyl, deuterated C₁₋₆ alkyl, C₁₋₆ alkoxy, halogenated C₁₋₆ alkoxy, C₃₋₆ cycloalkyl, halogenated C₃₋₆ cycloalkyl, deuterated C₃₋₆ cycloalkyl, 4-to 10-membered heterocyclyl, and any combination thereof. In the present disclosure, unless otherwise specified, any one end of the general formulas provided above may be connected to R_{w} of the compound of Formula (II), while the other end is connected to R_{w3}. The number of carbon atoms of each of the above general formulas (excluding the number of carbon atoms in the Rₐ and R_{b} groups), namely the sum of n1+n2*m3, the sum of n1+n2*m3+n3*m4, the sum of n1+n2*m3+n3*m4+n4*m5, each independently is an integer less than or equal to 20.

In some embodiments, R_{w1} of the compound of Formula (II) represents —C₁₋₁₅ alkylene-, wherein any one or more hydrogens of said C₁₋₁₅ alkylene are optionally replaced by a substituent selected from: deuterium, hydroxy, amino, mercapto, nitro, halogen, cyano, halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), C₁₋₆ alkoxy (e.g., C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O—, or CH₂ClCH₂—O—), C₃₋₆ cycloalkyl, halogenated C₃₋₆ cycloalkyl, deuterated C₃₋₆ cycloalkyl, 4-to 10-membered heterocyclyl, and any combination thereof.

In some embodiments, R_{w1} of the compound of Formula (II) represents the following groups:
—CH₂—, —(CH₂)₂—, —(CH₂)₃—, —(CH₂)₄—, —(CH₂)₅—, —(CH₂)₆—, —(CH₂)₇—, —(CH₂)₈—, —(CH₂)₉—, —(CH₂)₁₀—, —(CH₂)₁₁—, —(CH₂)₁₂—, —(CH₂)₁₃—, —(CH₂)₁₄—, or —(CH₂)₁₅—;
   wherein any one or more hydrogens of said groups are optionally replaced by a substituent selected from: deuterium, hydroxy, amino, mercapto, nitro, halogen, cyano, halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂—, or CH₂ClCH₂—), deuterated C₁₋₆ alkyl (e.g., perdeuterated C₁₋₆ alkyl, perdeuterated C₁₋₅ alkyl, or perdeuterated C₁₋₄ alkyl, such as CD₃, CD₃CD₂—, CD₃CD₂CD₂— etc.), C₁₋₆ alkoxy (e.g., C₁₋₄ alkoxy, such as methoxy, ethoxy, propoxy, isopropoxy, n-butoxy, sec-butoxy, or tert-butoxy), halogenated C₁₋₆ alkoxy (e.g., halogenated C₁₋₄ alkoxy, such as F₃C—O—, FCH₂—O—, F₂CH—O—, ClCH₂—O—, C₁₂CH—O—, CF₃CF₂—O—, CF₃CHF—O—, CHF₂CF₂—O—, CHF₂CHF—O—, CF₃CH₂—O—, or CH₂ClCH₂—O—), C₃₋₆ cycloalkyl, halogenated C₃₋₆ cycloalkyl, deuterated C₃₋₆ cycloalkyl, 4-to 10-membered heterocyclyl, and any combination thereof.

In some embodiments, R_{w1} of the compound of Formula (II) represents a structure of the following Formulae:
—(C(Rₐ₈)(Rₐ₉)ₙ₁—(O(C(Rₐ₁₀)(Rₐ₁₁))ₙ₂)ₘ₃—;
—(C(Rₐ₈)(Rₐ₉))ₙ₁—(O(C(Rₐ₁₀)(Rₐ₁₁))ₙ₂)ₘ₃—(O(C(Rₐ₁₂)(Rₐ₁₃))ₙ₃)ₘ₄—;
—(C(Rₐ₈)(Rₐ₉))ₙ₁—(O(C(Rₐ₁₀)(Rₐ₁₁))ₙ₂)ₘ₃—(O(C(Rₐ₁₂)(Rₐ₁₃))ₙ₃)ₘ₄—(O(C(Rₐ₁₄)(Rₐ₁₅))ₙ₄)ₘ₅—;
—(C(Rₐ₈)(Rₐ₉))ₙ₁—((C(Rₐ₁₀)(Rₐ₁₁))ₙ₂₀)ₘ₃—;
—(C(Rₐ₈)(Rₐ₉))ₙ₁—((C(Rₐ₁₀)(Rₐ₁₁))ₙ₂₀)ₘ₃—((C(Rₐ₁₂)(Rₐ₁₃))ₙ₃₀)ₘ₄—;
—(C(Rₐ₈)(Rₐ₉))ₙ₁—((C(Rₐ₁₀)(Rₐ₁₁))ₙ₂₀)ₘ₃—((C(Rₐ₁₂)(Rₐ₁₃))ₙ₃₀)ₘ₄—((C(Rₐ₁₄)(Rₐ₁₅))ₙ₄₀)ₘ₅—;
—(C(Rₐ₈)(Rₐ₉))ₙ₁—(N(Rₐ₇)(C(Rₐ₁₀)(Rₐ₁₁))ₙ₂)ₘ₃—;
—(C(Rₐ₈)(Rₐ₉))ₙ₁—(N(Rₐ₇)(C(Rₐ₁₀)(Rₐ₁₁))ₙ₂)ₘ₃—(N(Rₐ₇)(C(Rₐ₁₂)(Rₐ₁₃))ₙ₃)ₘ₄—;
—(C(Rₐ₈)(Rₐ₉))ₙ₁—(N(Rₐ₇)(C(Rₐ₁₀)(Rₐ₁₁))ₙ₂)ₘ₃—(N(Rₐ₇)(C(Rₐ₁₂)(Rₐ₁₃))ₙ₃)ₘ₄—(N(Rₐ₇)(C(Rₐ₁₄)(Rₐ₁₅))ₙ₄)ₘ₅—;
—(C(Rₐ₈)(Rₐ₉))ₙ₁—((C(Rₐ₁₀)(Rₐ₁₁))ₙ₂N(Rₐ₇))ₘ₃—;
—(C(Rₐ₈)(Rₐ₉))ₙ₁—((C(Rₐ₁₀)(Rₐ₁₁))ₙ₂N(Rₐ₇))ₘ₃—((C(Rₐ₁₂)(Rₐ₁₃))ₙ₃N(Rₐ₇))ₘ₄—;
—(C(Rₐ₈)(Rₐ₉))ₙ₁—((C(Rₐ₁₀)(Rₐ₁₁))ₙ₂N(Rₐ₇))ₘ₃—((C(Rₐ₁₂)(Rₐ₁₃))ₙ₃N(Rₐ₇))ₘ₄—((C(Rₐ₁₄)(Rₐ₁₅))ₙ₄N(Rₐ₇))ₘ₅—;
—(C(Rₐ₈)(Rₐ₉))ₙ₁—(C(O)N(Rₐ₇)—(C(Rₐ₁₀)(Rₐ₁₁))ₙ₂)ₘ₃—;
—(C(Rₐ₈)(Rₐ₉))ₙ₁—(C(O)N(Rₐ₇)—(C(Rₐ₁₀)(Rₐ₁₁))ₙ₂)ₘ₃—(C(O)N(Rₐ₇)—(C(Rₐ₁₂)(Rₐ₁₃))ₙ₃)ₘ₄—;
—(C(Rₐ₈)(Rₐ₉))ₙ₁—(C(O)N(Rₐ₇)—(C(Rₐ₁₀)(Rₐ₁₁))ₙ₂)ₘ₃—(C(O)N(Rₐ₇)—(C(Rₐ₁₂)(Rₐ₁₃))ₙ₃)ₘ₄—(C(O)N(Rₐ₇)—(C(Rₐ₁₄)(Rₐ₁₅))ₙ₄)ₘ₅—;
—(C(Rₐ₈)(Rₐ₉))ₙ₁—((C(Rₐ₁₀)(Rₐ₁₁))ₙ₂—C(O)N(Rₐ₇))ₘ₃—;
—(C(Rₐ₈)(Rₐ₉))ₙ₁—((C(Rₐ₁₀)(Rₐ₁₁))ₙ₂—C(O)N(Rₐ₇))ₘ₃—((C(Rₐ₁₂)(Rₐ₁₃))ₙ₃—C(O)N(Rₐ₇))ₘ₄—;
—(C(Rₐ₈)(Rₐ₉))ₙ₁—((C(Rₐ₁₀)(Rₐ₁₁))ₙ₂—C(O)N(Rₐ₇))ₘ₃—((C(Rₐ₁₂)(Rₐ₁₃))ₙ₃—C(O)N(Rₐ₇))ₘ₄—((C(Rₐ₁₄)(Rₐ₁₅))ₙ₄—C(O)N(Rₐ₇))ₘ₅—;
—(C(Rₐ₈)(Rₐ₉))ₙ₁—(C(O)N(Rₐ₇)—(C(Rₐ₁₀)(Rₐ₁₁))ₙ₂)ₘ₃—(O—(C(Rₐ₁₂)(Rₐ₁₃))ₙ₃)ₘ₄—;
—(C(Rₐ₈)(Rₐ₉))ₙ₁—C(O)N(Rₐ₇)—(C(Rₐ₁₀)(Rₐ₁₁))ₙ₂—(O(C(Rₐ₁₂)(Rₐ₁₃))ₙ₃)ₘ₃—;
—(C(Rₐ₈)(Rₐ₉))ₙ₁—((C(Rₐ₁₀)(Rₐ₁₁))ₙ₂—C(O)N(Rₐ₇))ₘ₃—((C(Rₐ₁₂)(Rₐ₁₃))ₙ₃₀)ₘ₄—;
—(C(Rₐ₈)(Rₐ₉))ₙ₁—(C(Rₐ₁₀)(Rₐ₁₁))ₙ₂—C(O)N(Rₐ₇)—((C(Rₐ₁₂)(Rₐ₁₃))ₙ₃₀)ₘ₃—;
—(C(Rₐ₈)(Rₐ₉))ₙ₁—(N(Rₐ₇)C(O)—(C(Rₐ₁₀)(Rₐ₁₁))ₙ₂)ₘ₃—;
—(C(Rₐ₈)(Rₐ₉))ₙ₁—(N(Rₐ₇)C(O)—(C(Rₐ₁₀)(Rₐ₁₁))ₙ₂)ₘ₃—(O—(C(Rₐ₁₂)(Rₐ₁₃)))ₙ₃)ₘ₄—;
—(C(Rₐ₈)(Rₐ₉))ₙ₁—(N(Rₐ₇)C(O)—(C(Rₐ₁₀)(Rₐ₁₁))ₙ₂)ₘ₃—(O—(C(Rₐ₁₂)(Rₐ₁₃))ₙ₃)ₘ₄—(O—(C(Rₐ₁₄)(Rₐ₁₅))ₙ₄)ₘ₅—;
—(C(Rₐ₈)(Rₐ₉))ₙ₁—N(Rₐ₇)C(O)—(C(Rₐ₁₀)(Rₐ₁₁))ₙ₂—(O(C(Rₐ₁₂)(Rₐ₁₃))ₙ₃)ₘ₃—;
—(C(Rₐ₈)(Rₐ₉))ₙ₁—N(Rₐ₇)C(O)N(Rₐ₇)—(C(Rₐ₁₀)(Rₐ₁₁))ₙ₂—; —(C(Rₐ₈)(Rₐ₉))ₙ₁—((C(Rₐ₁₀)(Rₐ₁₁))ₙ₂—N(Rₐ₇)C(O))ₘ₃—;
—(C(Rₐ₈)(Rₐ₉))ₙ₁—((C(Rₐ₁₀)(Rₐ₁₁))ₙ₂—N(Rₐ₇)C(O))ₘ₃—((C(Rₐ₁₂)(Rₐ₁₃)))ₙ₃—O)ₘ₄—;
—(C(Rₐ₈)(Rₐ₉))ₙ₁—((C(Rₐ₁₀)(Rₐ₁₁))ₙ₂—N(Rₐ₇)C(O))ₘ₃—((C(Rₐ₁₂)(Rₐ₁₃))ₙ₃₀)ₘ₄—((C(Rₐ₁₄)(Rₐ₁₅))ₙ₄₀)ₘ₅—;
—(C(Rₐ₈)(Rₐ₉))ₙ₁—(C(Rₐ₁₀)(Rₐ₁₁))ₙ₂—N(Rₐ₇)C(O)—((C(Rₐ₁₂)(Rₐ₁₃))ₙ₃₀)ₘ₃—;
—(C(Rₐ₈)(Rₐ₉))ₙ₁-(arylene-(C(Rₐ₁₀)(Rₐ₁₁))ₙ₂)ₘ₃—;
—(C(Rₐ₈)(Rₐ₉))ₙ₁-(arylene-(C(Rₐ₁₀)(Rₐ₁₁))ₙ₂)ₘ₃-arylene-(C(Rₐ₁₂)(Rₐ₁₃))ₙ₃—;
—(C(Rₐ₈)(Rₐ₉))ₙ₁—((C(Rₐ₁₀)(Rₐ₁₁))ₙ₂-arylene)ₘ₃-;
—(C(Rₐ₈)(Rₐ₉))ₙ₁—((C(Rₐ₁₀)(Rₐ₁₁))ₙ₂-arylene)ₘ₃-(C(Rₐ₁₂)(Rₐ₁₃))ₙ₃-arylene-;
—(C(Rₐ₈)(Rₐ₉))ₙ₁-(heterocyclylene-(C(Rₐ₁₀)(Rₐ₁₁))ₙ₂)ₘ₃—;
—(C(Rₐ₈)(Rₐ₉))ₙ₁-(heterocyclylene-(C(Rₐ₁₀)(Rₐ₁₁))ₙ₂)ₘ₃-(heterocyclylene-(C(Rₐ₁₂)(Rₐ₁₃))ₙ₃)ₘ₄—;
—(C(Rₐ₈)(Rₐ₉))ₙ₁—((C(Rₐ₁₀)(Rₐ₁₁))ₙ₂-heterocyclylene)ₘ₃-;
—(C(Rₐ₈)(Rₐ₉))ₙ₁—((C(Rₐ₁₀)(Rₐ₁₁))ₙ₂-heterocyclylene)ₘ₃-((C(Rₐ₁₂)(Rₐ₁₃))ₙ₃-heterocyclylene)ₘ₄-;
—(C(Rₐ₈)(Rₐ₉))ₙ₁-(heteroarylene-(C(Rₐ₁₀)(Rₐ₁₁))ₙ₂)ₘ₃—;
—(C(Rₐ₈)(Rₐ₉))ₙ₁-(heteroarylene-(C(Rₐ₁₀)(Rₐ₁₁))ₙ₂)ₘ₃-(heteroarylene-(C(Rₐ₁₂)(Rₐ₁₃))ₙ₃)ₘ₄—;
—(C(Rₐ₈)(Rₐ₉))ₙ₁—((C(Rₐ₁₀)(Rₐ₁₁))ₙ₂-heteroarylene)ₘ₃-;
—(C(Rₐ₈)(Rₐ₉))ₙ₁—((C(Rₐ₁₀)(Rₐ₁₁))ₙ₂-heteroarylene)ₘ₃-((C(Rₐ₁₂)(Rₐ₁₃))ₙ₃-heteroarylene)ₘ₄-;
—(C(Rₐ₈)(Rₐ₉))ₙ₁-(cycloalkylene-(C(Rₐ₁₀)(Rₐ₁₁))ₙ₂)ₘ₃—;
—(C(Rₐ₈)(Rₐ₉))ₙ₁-(cycloalkylene-(C(Rₐ₁₀)(Rₐ₁₁))ₙ₂)ₘ₃-(cycloalkylene-(C(Rₐ₁₂)(Rₐ₁₃))ₙ₃)ₘ₄—;
—(C(Rₐ₈)(Rₐ₉))ₙ₁—((C(Rₐ₁₀)(Rₐ₁₁))ₙ₂-cycloalkylene)ₘ₃-, or
—(C(Rₐ₈)(Rₐ₉))ₙ₁—((C(Rₐ₁₀)(Rₐ₁₁))ₙ₂-cycloalkylene)ₘ₃-((C(Rₐ₁₂)(Rₐ₁₃))ₙ₃-cycloalkylene)ₘ₄-;
wherein each Rₐ₇ independently represents H or C₁₋₃ alkyl;
the cycloalkylene (e.g., C₃₋₂₀ cycloalkylene), arylene (e.g., C₅₋₂₀ arylene), heterocyclylene (e.g., 4-to 20-membered heterocyclylene), and heteroarylene (e.g., 5-to 20-membered heteroarylene) are each independently optionally substituted with a substituent selected from the group consisting of deuterium, hydroxy, amino, mercapto, nitro, halogen, cyano, oxo, C₁₋₆ alkyl, halogenated C₁₋₆ alkyl, deuterated C₁₋₆ alkyl, C₁₋₆ alkoxy, halogenated C₁₋₆ alkoxy, C₃₋₆ cycloalkyl, halogenated C₃₋₆ cycloalkyl, deuterated C₃₋₆ cycloalkyl, 4-to 10-membered heterocyclyl, and any combination thereof;
Rₐ₈, Rₐ₉, Rₐ₁₀, Rₐ₁₁, Rₐ₁₂, Rₐ₁₃, Rₐ₁₄, and Rₐ₁₅ each independently represent H, deuterium, hydroxy, amino, mercapto, nitro, halogen, cyano, halogenated C₁₋₆ alkyl, deuterated C₁₋₆ alkyl, C₁₋₆ alkoxy, halogenated C₁₋₆ alkoxy, C₃₋₆ cycloalkyl, halogenated C₃₋₆ cycloalkyl, deuterated C₃₋₆ cycloalkyl, 4-to 10-membered heterocyclyl, and any combination thereof, and
n1, n2, n3, n4, m3, m4, and m5 each independently represent an integer of 1, 2, 3, 4, 5, 6, 7, 8, 9, or 10. The total number of carbon atoms of alkylene in each of the above general formulas, namely the sum of n1+n2*m3, the sum of n1+n2*m3+n3*m4, the sum of n1+n2*m3+n3*m4+n4*m5, each independently is an integer less than or equal to 20.

In some embodiments, examples of the cycloalkylene in the general formula structure represented by R_{w1} of the compound of Formula (II) include, but are not limited to, C₃₋₂₀ cycloalkylene, C₃₋₁₅cycloalkylene, and C₃₋₁₁cycloalkylene, such as cyclopropylene, cyclobutylene, cyclopentylene, cyclopentenylene, cyclohexylene, cyclohexenylene, cycloheptylene, cyclooctylene, decalinylene, octahydropentalenylene, octahydro-1H-indenylene, 2,3-dihydro-1H-indenylene, spiro-cycloalkylene (e.g., C₅₋₁₅ spiro-cycloalkylene), adamantanylene, noradamantanylene, and norbornylene. The cycloalkylene is optionally substituted with a substituent selected from the group consisting of deuterium, hydroxy, amino, mercapto, nitro, halogen, cyano, oxo, C₁₋₆ alkyl, halogenated C₁₋₆ alkyl, deuterated C₁₋₆ alkyl, C₁₋₆ alkoxy, halogenated C₁₋₆ alkoxy, C₃₋₆ cycloalkyl, halogenated C₃₋₆ cycloalkyl, deuterated C₃₋₆ cycloalkyl, 4-to 10-membered heterocyclyl, and any combination thereof.

In some embodiments, examples of the arylene in the general formula structure represented by R_{w1} of the compound of Formula (II) include, but are not limited to, C₅₋₂₀ arylene, C₆₋₂₀ arylene, C₅₋₁₅ arylene, and C₆₋₁₅ arylene, such as phenylene or naphthylene. The arylene is optionally substituted with a substituent selected from the group consisting of deuterium, hydroxy, amino, mercapto, nitro, halogen, cyano, C₁₋₆ alkyl, halogenated C₁₋₆ alkyl, deuterated C₁₋₆ alkyl, C₁₋₆ alkoxy, halogenated C₁₋₆ alkoxy, C₃₋₆ cycloalkyl, halogenated C₃₋₆ cycloalkyl, deuterated C₃₋₆ cycloalkyl, 4-to 10-membered heterocyclyl, and any combination thereof.

In some embodiments, examples of the heterocyclylene in the general formula structure represented by R_{w1} of the compound of Formula (II) include, but are not limited to, 4-to 20-membered, 4-to 15-membered, 4-to 12-membered, 4-to 11-membered, 4-to 10-membered, 4-to 9-membered, 4-to 8-membered, 4-to 7-membered, 4-to 6-membered, 5-to 15-membered, and 5-to 9-membered heterocyclylene, such as azetidinylene, oxetanylene, pyrrolidinylene, imidazolidinylene, pyrazolidylene, tetrahydrofuranylene, tetrahydropyranylene, tetrahydrothienylene, tetrahydrothiopyranylene, oxazolidinylene, thiazolidinylene, piperidinylene, piperazinylene, morpholinylene, thiomorpholinylene, azacycloheptanylene, azacyclooctylene, dioxacyclohexylene, azacycloheptanylene, azacyclooctylene, diazacycloheptanylene, diazacyclooctylene, 6-azabicyclo[3.1.1]heptanylene, 2,5-diazabicyclo[2.2.1]heptanylene, 3,6-diazabicyclo[3.1.1]heptanylene, 3-azabicyclo[3.2.1]octanylene, 3,8-diazabicyclo[3.2.1]octanylene, 3,8-diazabicyclo[3.2.1]octanylene, 2,5-diazabicyclo[2.2.2]octanylene, and 5-to 20-membered azaspirocycloalkylene. The heterocyclylene is optionally substituted with a substituent selected from the group consisting of deuterium, hydroxy, amino, mercapto, nitro, halogen, cyano, oxo, C₁₋₆ alkyl, halogenated C₁₋₆ alkyl, deuterated C₁₋₆ alkyl, C₁₋₆ alkoxy, halogenated C₁₋₆ alkoxy, C₃₋₆ cycloalkyl, halogenated C₃₋₆ cycloalkyl, deuterated C₃₋₆ cycloalkyl, 4-to 10-membered heterocyclyl, and any combination thereof.

In some embodiments, examples of the heteroarylene in the general formula structure represented by R_{w1} of the compound of Formula (II) include, but are not limited to, 5-to 20-membered heteroarylene, 5-to 15-membered, 5-to 10-membered, 5-to 9-membered, 5-to 8-membered, 5-to 7-membered, and 5-to 6-membered heteroarylene, e.g., oxazolylene, isoxazolylene, oxadiazolylene, thienylene, thiazolylene, isothiazolylene, thiadiazolylene, pyrrolylene, imidazolylene, pyrazolylene, triazolylene, pyridylene, pyrimidinylene, pyridazinylene, pyrazinylene, indolylene, isoindolylene, benzofuranylene, isobenzofuranylene, benzothienylene, indazolylene, benzimidazolylene, benzoxazolylene, benzisoxazolylene, benzothiazolylene, benzisothiazolylene, benzotriazolylene, benzo[2,1,3]oxadiazolylene, benzo[2,1,3]thiadiazolylene, benzo[1,2,3]thiadiazolylene, quinolinylene, isoquinolinylene, naphthyridinylene, cinnolinylene, quinazolinylene, quinoxalinylene, phthalazinylene, pyrazolo[1,5-a]pyridylene, pyrazolo[1,5-a]pyrimidinylene, imidazo[1,2-a]pyridylene, 1H-pyrrolo[3,2-b]pyridylene, 1H-pyrrolo[2,3-b]pyridylene, pyrrolo[2,1-b]thiazolylene, and imidazo[2,1-b]thiazolylene. The heteroarylene is optionally substituted with a substituent selected from the group consisting of deuterium, hydroxy, amino, mercapto, nitro, halogen, cyano, C₁₋₆ alkyl, halogenated C₁₋₆ alkyl, deuterated C₁₋₆ alkyl, C₁₋₆ alkoxy, halogenated C₁₋₆ alkoxy, C₃₋₆ cycloalkyl, halogenated C₃₋₆ cycloalkyl, deuterated C₃₋₆ cycloalkyl, 4-to 10-membered heterocyclyl, and any combination thereof.

In some embodiments, examples of R_{w1} of the compound of Formula (II) include, but are not limited to, the following groups:
—O—CH₂—, —O—(CH₂)₂—, —O—(CH₂)₃—, —O—(CH₂)₄—, —O—(CH₂)₅—, —O—(CH₂)₆—, —O—(CH₂)₇—, —O—(CH₂)₈—, —O—(CH₂)₉—, —O—(CH₂)₁₀—, —(CH₂)₁—O—, —(CH₂)₂—O—, —(CH₂)₃—O—, —(CH₂)₄—O—, —(CH₂)₅—O—, —(CH₂)₆—O—, —(CH₂)₇—O—, —(CH₂)₈—O—, (CH₂)₉—O—, —(CH₂)₁₀—O—, —CH₂—O—CH₂—, —CH₂—O—(CH₂)₂—, —(CH₂)₁—O—(CH₂)₃—, —(CH₂)₁—O—(CH₂)₄—, —(CH₂)₁—O—(CH₂)₅—, —(CH₂)₁—O—(CH₂)₆—, —(CH₂)₁—O—(CH₂)₇—, —(CH₂)₁—O—(CH₂)₈—, —(CH₂)₁—O—(CH₂)₉—, —(CH₂)₁—O—(CH₂)₁₀—, —(CH₂)₂—O—(CH₂)₁—, —(CH₂)₂—O—(CH₂)₂—, —(CH₂)₂—O—(CH₂)₃—, —(CH₂)₂—O—(CH₂)₄—, —(CH₂)₂—O—(CH₂)₅—, —(CH₂)₂—O—(CH₂)₆—, —(CH₂)₂—O—(CH₂)₇—, —(CH₂)₂—O—(CH₂)₈—, —(CH₂)₂—O—(CH₂)₉—, —(CH₂)₂—O—(CH₂)₁₀—, —(CH₂)₃—O—(CH₂)₁—, —(CH₂)₃—O—(CH₂)₂—, —(CH₂)₃—O—(CH₂)₃—, —(CH₂)₃—O—(CH₂)₄—, —(CH₂)₃—O—(CH₂)₅—, —(CH₂)₃—O—(CH₂)₆—, —(CH₂)₃—O—(CH₂)₇—, —(CH₂)₄—O—(CH₂)₁—, —(CH₂)₄—O—(CH₂)₂—, —(CH₂)₄—O—(CH₂)₃—, —(CH₂)₄—O—(CH₂)₄—, —(CH₂)₄—O—(CH₂)₅—, —(CH₂)₄—O—(CH₂)₆—, —(CH₂)₅—O—(CH₂)₁—, —(CH₂)₅—O—(CH₂)₂—, —(CH₂)₅—O—(CH₂)₃—, —(CH₂)₅—O—(CH₂)₄—, —(CH₂)₅—O—(CH₂)₅—, —(CH₂)₆—O—(CH₂)₁—, —(CH₂)₆—O—(CH₂)₂—, —(CH₂)₆—O—(CH₂)₃—, —(CH₂)₆—O—(CH₂)₄—, —(CH₂)₇—O—(CH₂)₁—, —(CH₂)₇—O—(CH₂)₂—, —(CH₂)₇—O—(CH₂)₃—, —(CH₂)₈—O—(CH₂)₁—, —(CH₂)₈—O—(CH₂)₂—, —CH(CH₃)—O—(CH₂)₁—, —CH(CH₃)—O—(CH₂)₂—, —CH(CH₃)—O—(CH₂)₃—, —CH(CH₃)—O—(CH₂)₄—, —CH(CH₃)—O—(CH₂)₅—, —CH(CH₃)—O—(CH₂)₆—, —CH(CH₃)—O—(CH₂)₇—, —CH(CH₃)—O—(CH₂)₈—, —CH(CH₃)—O—(CH₂)₉—, —CH(CH₃)—O—(CH₂)₁₀—, —(O(CH₂)₂)₂—, —(O(CH₂)₂)₃—, —(O(CH₂)₂)₄—, —(O(CH₂)₂)₅—, —(O(CH₂)₂)₆—, —(O(CH₂)₂)₇—, —CH₂—(O(CH₂)₂)₂—, —CH₂—(O(CH₂)₂)₃—, —CH₂—(O(CH₂)₂)₄—, —CH₂—(O(CH₂)₂)₅—, —CH₂—(O(CH₂)₂)₆—, —CH₂—(O(CH₂)₂)₇—, —CH₂—(O(CH₂)₂)₁—OCH₂—, —CH₂—(O(CH₂)₂)₂—OCH₂—, —CH₂—(O(CH₂)₂)₃—OCH₂—, —CH₂—(O(CH₂)₂)₄—OCH₂—, —CH₂—(O(CH₂)₂)₅—OCH₂—, —(CH₂)₂—(O(CH₂)₂)₂—, —(CH₂)₂—(O(CH₂)₂)₃—, —(CH₂)₂—(O(CH₂)₂)₄—, —(CH₂)₂—(O(CH₂)₂)₅—, —(CH₂)₂—(O(CH₂)₂)₆—, —(CH₂)₃—(O(CH₂)₂)₂—, —(CH₂)₃—(O(CH₂)₂)₃—, —(CH₂)₃—(O(CH₂)₂)₄—, —(CH₂)₃—(O(CH₂)₂)₅—, —(CH₂)₄—(O(CH₂)₂)₂—, —(CH₂)₄—(O(CH₂)₂)₃—, —(CH₂)₄—(O(CH₂)₂)₄—, —(CH₂)₄—(O(CH₂)₂)₅—, —CH₂—(O(CH₂)₃)₂—, —CH₂—(O(CH₂)₃)₃—, —CH₂—(O(CH₂)₃)₄—, (CH₂)₂—(O(CH₂)₃)₂—, —(CH₂)₂—(O(CH₂)₃)₃—, —(CH₂)₂—(O(CH₂)₃)₄—, —(CH₂)₃—(O(CH₂)₃)₂—, —(CH₂)₃—(O(CH₂)₃)₃—, —(CH₂)₃—(O(CH₂)₃)₄—, —CH₂—O—(CH₂)₂—O—(CH₂)₃—, —CH₂—(O(CH₂)₂)₂—(O(CH₂)₃)₂—, (CH₂)₂—O—(CH₂)₂—O—(CH₂)₃—, —(CH₂)₂—(O(CH₂)₂)₂—(O(CH₂)₃)₂—, —(CH₂)₃—O—(CH₂)₂—O—(CH₂)₃—, —(CH₂)₃—(O(CH₂)₂)₂—(O(CH₂)₃)₂—, —CH₂—O—(CH₂)₃—O—(CH₂)₂—, —CH₂—(O(CH₂)₃)₂—(O(CH₂)₂)₂—, —(CH₂)₂—O—(CH₂)₃—O—(CH₂)₂—, —(CH₂)₂—(O(CH₂)₃)₂—(O(CH₂)₂)₂—, —(CH₂)₃—O—(CH₂)₃—O—(CH₂)₂—, —(CH₂)₃—(O(CH₂)₃)₂—(O(CH₂)₂)₂—, —CH₂—O—(CH₂)₂—O—CH₂—, —(CH₂)₂—O—(CH₂)₂—O—CH₂—, —(CH₂)₂—(O(CH₂)₂)₂—O—(CH₂)₃—, —(CH₂)₂—(O(CH₂)₂)₃—O—(CH₂)₃—, —(CH₂)₂—(O(CH₂)₂)₄—O—(CH₂)₃—, —(CH₂)₅—(O(CH₂)₂)₂—O—(CH₂)₅—, —(CH₂)₅—(O(CH₂)₂)₂—O—(CH₂)₆—, —(CH₂)₁—N(Rₐ₇)—(CH₂)₁—, —(CH₂)₁—N(Rₐ₇)—(CH₂)₂—, —(CH₂)₁—N(Rₐ₇)—(CH₂)₃—, —(CH₂)₁—N(Rₐ₇)—(CH₂)₄—, —(CH₂)₁—N(Rₐ₇)—(CH₂)₅—, —(CH₂)₁—N(Rₐ₇)—(CH₂)₆—, —(CH₂)₁—N(Rₐ₇)—(CH₂)₇—, —(CH₂)₁—N(Rₐ₇)—(CH₂)₈—, —(CH₂)₁—N(Rₐ₇)—(CH₂)₉—, —(CH₂)₁—N(Rₐ₇)—(CH₂)₁₀—, —N(Rₐ₇)—(CH₂)₁—, —N(Rₐ₇)—(CH₂)₂—, —N(Rₐ₇)—(CH₂)₃—, —N(Rₐ₇)—(CH₂)₄—, —N(Rₐ₇)—(CH₂)₅—, —N(Rₐ₇)—(CH₂)₆—, —N(Rₐ₇)—(CH₂)₇—, —N(Rₐ₇)—(CH₂)₈—, —N(Rₐ₇)—(CH₂)₉—, —N(Rₐ₇)—(CH₂)₁₀—, —(CH₂)₂—N(Rₐ₇)—(CH₂)₁—, —(CH₂)₂—N(Rₐ₇)—(CH₂)₂—, —(CH₂)₂—N(Rₐ₇)—(CH₂)₃—, —(CH₂)₂—N(Rₐ₇)—(CH₂)₄—, —(CH₂)₂—N(Rₐ₇)—(CH₂)₅—, —(CH₂)₂—N(Rₐ₇)—(CH₂)₆—, —(CH₂)₂—N(Rₐ₇)—(CH₂)₇—, —(CH₂)₂—N(Rₐ₇)—(CH₂)₈—, —(CH₂)₂—N(Rₐ₇)—(CH₂)₉—, —(CH₂)₂—N(Rₐ₇)—(CH₂)₁₀—, —(CH₂)₃—N(Rₐ₇)—(CH₂)₁—, —(CH₂)₃—N(Rₐ₇)—(CH₂)₂—, —(CH₂)₃—N(Rₐ₇)—(CH₂)₃—, —(CH₂)₄—N(Rₐ₇)—(CH₂)₁—, —(CH₂)₄—N(Rₐ₇)—(CH₂)₂—, —(CH₂)₄—N(Rₐ₇)—(CH₂)₃—, —(CH₂)₄—N(Rₐ₇)—(CH₂)₄—, —(CH₂)₅—N(Rₐ₇)—(CH₂)₁—, —(CH₂)₅—N(Rₐ₇)—(CH₂)₂—, —(CH₂)₅—N(Rₐ₇)—(CH₂)₃—, —(CH₂)₅—N(Rₐ₇)—(CH₂)₄—, —(CH₂)₅—N(Rₐ₇)—(CH₂)₅—, —(CH₂)₆—N(Rₐ₇)—(CH₂)₁—, —(CH₂)₆—N(Rₐ₇)—(CH₂)₂—, —(CH₂)₆—N(Rₐ₇)—(CH₂)₃—, —(CH₂)₇—N(Rₐ₇)—(CH₂)₁—, —(CH₂)₇—N(Rₐ₇)—(CH₂)₂—, —(CH₂)₇—N(Rₐ₇)—(CH₂)₃—, —(CH₂)₈—N(Rₐ₇)—(CH₂)₁—, —(CH₂)₈—N(Rₐ₇)—(CH₂)₂—, —(CH₂)₈—N(Rₐ₇)—(CH₂)₃—, —CH(CH₃)—N(Rₐ₇)—(CH₂)₁—, —CH(CH₃)—N(Rₐ₇)—(CH₂)₂—, —CH(CH₃)—N(Rₐ₇)—(CH₂)₃—, —CH(CH₃)—N(Rₐ₇)—(CH₂)₄—, —CH(CH₃)—N(Rₐ₇)—(CH₂)₅—, —CH(CH₃)—N(Rₐ₇)—(CH₂)₆—, —CH(CH₃)—N(Rₐ₇)—(CH₂)₇—, —CH(CH₃)—N(Rₐ₇)—(CH₂)₈—, —CH(CH₃)—N(Rₐ₇)—(CH₂)₉—, —CH(CH₃)—N(Rₐ₇)—(CH₂)₁₀—, —C(O)NHCH₂—, —C(O)NH(CH₂)₂—, —C(O)NH(CH₂)₃—, —C(O)NH(CH₂)₄—, —C(O)NH(CH₂)₅—, —CH₂C(O)NHCH₂—, —(CH₂)₂C(O)NH(CH₂)₂—, —(CH₂)₂C(O)NH(CH₂)₃—, —(CH₂)₂C(O)NH(CH₂)₄—, —(CH₂)₂C(O)NH(CH₂)₅—, —(CH₂)₃C(O)NH(CH₂)₃—, —(CH₂)₃C(O)NH(CH₂)₄—, —(CH₂)₄C(O)NH(CH₂)₄—, —(CH₂)₅C(O)NH(CH₂)₅—, —(CH₂)₆C(O)NH(CH₂)₇—, —(CH₂), C(O)NH(CH₂)₆—, —(CH₂)—C(O)NH(CH₂)₇—, —(CH₂)₂C(O)NH(CH₂)₂—O—(CH₂)₂—, —NHC(O)CH₂—, —NHC(O)(CH₂)₂—, —NHC(O)(CH₂)₃—, —NHC(O)(CH₂)₄—, —NHC(O)(CH₂)₅—, —CH₂NHC(O)CH₂—, —(CH₂)₂NHC(O)(CH₂)₂—, —(CH₂)₂NHC(O)(CH₂)₃—, —(CH₂)₂NHC(O)(CH₂)₄—, (CH₂)₂NHC(O)(CH₂)₅—, —(CH₂)₃NHC(O)(CH₂)₃—, —(CH₂)₃NHC(O)(CH₂)₄—, —(CH₂)₄NHC(O)(CH₂)₄—, —(CH₂)₅NHC(O)(CH₂)₅—, —(CH₂)₆NHC(O)(CH₂)₇—, —(CH₂)₆NHC(O)(CH₂)₆—, —(CH₂)₇NHC(O)(CH₂)₇—, (CH₂)₄NHC(O)(CH₂)₈—, —(CH₂)₂NHC(O)(CH₂)₂—O—(CH₂)₂—, —(CH₂)₄NHC(O)CH₂—,-phenylene-CH₂—, phenylene-(CH₂)₂—,-phenylene-(CH₂)₃—,-phenylene-(CH₂)₄—,-phenylene-(CH₂)₅—,-phenylene-(CH₂)₆—,-phenylene-(CH₂)₇—,-phenylene-(CH₂)₈—, —CH₂-phenylene-CH₂—, —CH₂-phenylene-(CH₂)₂—, —CH₂-phenylene-(CH₂)₃—, —CH₂-phenylene-(CH₂)₄—, —CH₂-phenylene-(CH₂)₅—, —CH₂-phenylene-(CH₂)₆—, —CH₂-phenylene-(CH₂)₇—, —CH₂-phenylene-(CH₂)₈—, —(CH₂)₂-phenylene-(CH₂)₁—, —(CH₂)₂-phenylene-(CH₂)₂—, —(CH₂)₂-phenylene-(CH₂)₃—, —(CH₂)₂-phenylene-(CH₂)₄—, —(CH₂)₂-phenylene-(CH₂)₅—, —(CH₂)₂-phenylene-(CH₂)₆—, —(CH₂)₂-phenylene-(CH₂)₇—, (CH₂)₂-phenylene-(CH₂)₈—, —(CH₂)₃-phenylene-CH₂—, —(CH₂)₃-phenylene-(CH₂)₂—, —(CH₂)₃-phenylene-(CH₂)₃—, —(CH₂)₃-phenylene-(CH₂)₄—, —(CH₂)₃-phenylene-(CH₂)₅—, —(CH₂)₃-phenylene-(CH₂)₆—, —(CH₂)₃-phenylene-(CH₂)₇—, (CH₂)₃-phenylene-(CH₂)₈—, —(CH₂)₄-phenylene-CH₂—, —(CH₂)₄-phenylene-(CH₂)₂—, —(CH₂)₄-phenylene-(CH₂)₃—, —(CH₂)₄-phenylene-(CH₂)₄—, —(CH₂)₄-phenylene-(CH₂)₅—, —(CH₂)₄-phenylene-(CH₂)₆—, —(CH₂)₄-phenylene-(CH₂)₇—, (CH₂)₄-phenylene-(CH₂)₈—, —(CH₂)₅-phenylene-(CH₂)₁—, —(CH₂)₅-phenylene-(CH₂)₂—, —(CH₂)₅-phenylene-(CH₂)₃—, —(CH₂)₅-phenylene-(CH₂)₄—, —(CH₂)₅-phenylene-(CH₂)₅—, —(CH₂)₅-phenylene-(CH₂)₆—, —(CH₂)₅-phenylene-(CH₂)₇—, —(CH₂)₅-phenylene-(CH₂)₈—, —(CH₂)₆-phenylene-(CH₂)₁—, —(CH₂)₆-phenylene-(CH₂)₂—, —(CH₂)₆-phenylene-(CH₂)₃—, —(CH₂)₆-phenylene-(CH₂)₄—, —(CH₂)₆-phenylene-(CH₂)₅—, —(CH₂)₆-phenylene-(CH₂)₆—, (CH₂)₆-phenylene-(CH₂)₇—, —(CH₂)₆-phenylene-(CH₂)₈—, —(CH₂)₇-phenylene-(CH₂)₁—, —(CH₂)₇-phenylene-(CH₂)₂—, —(CH₂)₇-phenylene-(CH₂)₃—, —(CH₂)₇-phenylene-(CH₂)₄—, —(CH₂)₇-phenylene-(CH₂)₈—, —(CH₂)₈-phenylene-CH₂—, —(CH₂)₈-phenylene-(CH₂)₂—, —(CH₂)₈-phenylene-(CH₂)₃—, —(CH₂)₈-phenylene-(CH₂)₄—, —(CH₂)₈-phenylene-(CH₂)₅—, —(CH₂)₈-phenylene-(CH₂)₆—, —(CH₂)₈-phenylene-(CH₂)₇—, —N(Rₐ₇)—CH₂-phenylene-CH₂—, —N(Rₐ₇)—CH₂-phenylene-(CH₂)₂—, —N(Rₐ₇)—CH₂-phenylene-(CH₂)₃—, —N(Rₐ₇)—CH₂-phenylene-(CH₂)₄—, —N(Rₐ₇)—CH₂-phenylene-(CH₂)₅—, —N(Rₐ₇)—CH₂-phenylene-(CH₂)₆—, —N(Rₐ₇)—CH₂-phenylene-(CH₂)₇—, —N(Rₐ₇)—CH₂-phenylene-(CH₂)₈—, —CH₂—N(Rₐ₇)—CH₂-phenylene-CH₂—, —CH₂—N(Rₐ₇)—CH₂-phenylene-(CH₂)₂—, —CH₂—N(Rₐ₇)—CH₂-phenylene-(CH₂)₃—, —CH₂—N(Rₐ₇)—CH₂-phenylene-(CH₂)₄—, —CH₂—N(Rₐ₇)—CH₂-phenylene-(CH₂)₅—, —CH₂—N(Rₐ₇)—CH₂-phenylene-(CH₂)₆—, —CH₂—N(Rₐ₇)—CH₂-phenylene-(CH₂)₇—, —CH₂—N(Rₐ₇)—CH₂-phenylene-(CH₂)₈—, —CH₂—N(Rₐ₇)—(CH₂)₂-phenylene-CH₂—, —CH₂—N(Rₐ₇)—(CH₂)₂-phenylene-(CH₂)₂—, —CH₂—N(Rₐ₇)—(CH₂)₂- phenylene-(CH₂)₃—, —CH₂—N(Rₐ₇)—(CH₂)₂-phenylene-(CH₂)₄—, —CH₂—N(Rₐ₇)—(CH₂)₂-phenylene-(CH₂)₅—, —CH₂—N(Rₐ₇)—(CH₂)₂-phenylene-(CH₂)₆—, —CH₂—N(Rₐ₇)—(CH₂)₂-phenylene-(CH₂)₇—, —CH₂—N(Rₐ₇)—(CH₂)₂-phenylene-(CH₂)₈—, —(CH₂)₂—N(Rₐ₇)—CH₂-phenylene-CH₂—, —(CH₂)₂—N(Rₐ₇)—CH₂-phenylene-(CH₂)₂—, —(CH₂)₂—N(Rₐ₇)—CH₂-phenylene-(CH₂)₃—, (CH₂)₂—N(Rₐ₇)—CH₂-phenylene-(CH₂)₄—, —(CH₂)₂—N(Rₐ₇)—CH₂-phenylene-(CH₂)₅—, —(CH₂)₂—N(Rₐ₇)—CH₂-phenylene-(CH₂)₆—, —(CH₂)₂—N(Rₐ₇)—CH₂-phenylene-(CH₂)₇—, —(CH₂)₂—N(Rₐ₇)—CH₂-phenylene-(CH₂)₈—, —(CH₂)₃—N(Rₐ₇)—CH₂-phenylene-CH₂—, —(CH₂)₃—N(Rₐ₇)—CH₂-phenylene-(CH₂)₂—, —(CH₂)₃—N(Rₐ₇)—CH₂-phenylene-(CH₂)₃—, (CH₂)₃—N(Rₐ₇)—CH₂-phenylene-(CH₂)₈—, —(CH₂)₄—N(Rₐ₇)—CH₂-phenylene-CH₂—, —(CH₂)₄—N(Rₐ₇)—CH₂- phenylene-(CH₂)₂—, —(CH₂)₄—N(Rₐ₇)—CH₂-phenylene-(CH₂)₃—, —(CH₂)₄—N(Rₐ₇)—CH₂-phenylene-(CH₂)₈—, —(CH₂)₅—N(Rₐ₇)—CH₂-phenylene-(CH₂)₃—, —(CH₂)₅—N(Rₐ₇)—CH₂-phenylene-(CH₂)₈—, —(CH₂)₆—N(Rₐ₇)—CH₂-phenylene-(CH₂)₃—, —(CH₂)₆—N(Rₐ₇)—CH₂-phenylene-(CH₂)₈—, —(CH₂)₇—N(Rₐ₇)—CH₂-phenylene-(CH₂)₃—, —(CH₂)₈—N(Rₐ₇)—CH₂-phenylene- CH₂—, (CH₂)₈—N(Rₐ₇)—CH₂-phenylene-(CH₂)₂—, —(CH₂)₈—N(Rₐ₇)—CH₂-phenylene-(CH₂)₃—, —(CH₂)₈—N(Rₐ₇)—CH₂-phenylene-(CH₂)₄—, —(CH₂)₈—N(Rₐ₇)—CH₂-phenylene-(CH₂)₅—, —(CH₂)₈—N(Rₐ₇)—CH₂-phenylene-(CH₂)₆—,- piperidinylene-CH₂—,-piperidinylene-(CH₂)₂—,-piperidinylene-(CH₂)₃—,-piperidinylene-(CH₂)₄—, piperidinylene-(CH₂)₅—,-piperidinylene-(CH₂)₆—,-piperidinylene-(CH₂)₇—,-piperidinylene-(CH₂)₈—, —CH₂-piperidinylene-CH₂—, —CH₂-piperidinylene-(CH₂)₂—, —CH₂-piperidinylene-(CH₂)₃—, —CH₂-piperidinylene-(CH₂)₄—, —CH₂-piperidinylene-(CH₂)₅—, —CH₂-piperidinylene-(CH₂)₆—, —CH₂-piperidinylene-(CH₂)₇—, —CH₂-piperidinylene-(CH₂)₈—, —(CH₂)₂-piperidinylene-(CH₂)₁—, —(CH₂)₂-piperidinylene-(CH₂)₂—, —(CH₂)₂-piperidinylene-(CH₂)₃—, —(CH₂)₂-piperidinylene-(CH₂)₄—, —(CH₂)₂-piperidinylene-(CH₂)₅—, —(CH₂)₂-piperidinylene-(CH₂)₆—, —(CH₂)₂-piperidinylene-(CH₂)₇—, —(CH₂)₂-piperidinylene-(CH₂)₈—, —(CH₂)₃-piperidinylene-CH₂—, —(CH₂)₃-piperidinylene-(CH₂)₂—, —(CH₂)₃-piperidinylene-(CH₂)₃—, —(CH₂)₃-piperidinylene-(CH₂)₄—, —(CH₂)₃-piperidinylene-(CH₂)₅—, —(CH₂)₃-piperidinylene-(CH₂)₆—, —(CH₂)₃-piperidinylene-(CH₂)₇—, —(CH₂)₃-piperidinylene-(CH₂)₈—, —(CH₂)₄-piperidinylene-CH₂—, —(CH₂)₄-piperidinylene-(CH₂)₂—, —(CH₂)₄-piperidinylene-(CH₂)₃—, —(CH₂)₄-piperidinylene-(CH₂)₄—, —(CH₂)₄-piperidinylene-(CH₂)₅—, —(CH₂)₄-piperidinylene-(CH₂)₆—, —(CH₂)₄-piperidinylene-(CH₂)₇—, —(CH₂)₄-piperidinylene-(CH₂)₈—, —(CH₂)₅-piperidinylene-(CH₂)₁—, —(CH₂)₅-piperidinylene-(CH₂)₂—, —(CH₂)₅-piperidinylene-(CH₂)₃—, —(CH₂)₅-piperidinylene-(CH₂)₄—, —(CH₂)₅-piperidinylene-(CH₂)₅—, —(CH₂)₅—(CH₂)₅-piperidinylene-(CH₂)₈—, piperidinylene-(CH₂)₆—, —(CH₂)₅-piperidinylene-(CH₂)₇—, —(CH₂)₆—(CH₂)₆-piperidinylene-(CH₂)₂—, —(CH₂)₆—, —(CH₂)₆-piperidinylene-(CH₂)₃—, piperidinylene-(CH₂)₁—, (CH₂)₆—, —(CH₂)₆-piperidinylene-(CH₂)₆—, —(CH₂)₆-piperidinylene-(CH₂)₅—, piperidinylene-(CH₂)₄—, (CH₂)₇-piperidinylene-(CH₂)₁—, —(CH₂)₇—, —(CH₂)₆-piperidinylene-(CH₂)₈—, piperidinylene-(CH₂)₇—, (CH₂)₇-piperidinylene-(CH₂)₄—, —(CH₂)₇—, —(CH₂)₇-piperidinylene-(CH₂)₃—, piperidinylene-(CH₂)₂—, piperidinylene-(CH₂)₈—, —(CH₂)₈-piperidinylene-CH₂—, —(CH₂)₈-piperidinylene-(CH₂)₂—, —(CH₂)₈-piperidinylene-(CH₂)₃—, —(CH₂)₈-piperidinylene-(CH₂)₄—, —(CH₂)₈-piperidinylene-(CH₂)₅—, —(CH₂)₈-piperidinylene-(CH₂)₆—, —(CH₂)₈-piperidinylene-(CH₂)₇—, —N(Rₐ₇)—CH₂-piperidinylene-CH₂—, —N(Rₐ₇)—CH₂-piperidinylene-(CH₂)₂—, —N(Rₐ₇)—CH₂-piperidinylene-(CH₂)₃—, —N(Rₐ₇)—CH₂-piperidinylene-(CH₂)₄—, —N(Rₐ₇)—CH₂-piperidinylene-(CH₂)₅—, —N(Rₐ₇)—CH₂-piperidinylene-(CH₂)₆—, —N(Rₐ₇)—CH₂-piperidinylene-(CH₂)₇—, —N(Rₐ₇)—CH₂-piperidinylene-(CH₂)₈—, —CH₂—N(Rₐ₇)—CH₂-piperidinylene-CH₂—, —CH₂—N(Rₐ₇)—CH₂-piperidinylene-(CH₂)₂—, —CH₂—N(Rₐ₇)—CH₂-piperidinylene-(CH₂)₃—, —CH₂—N(Rₐ₇)—CH₂-piperidinylene-(CH₂)₄—, —CH₂—N(Rₐ₇)—CH₂-piperidinylene-(CH₂)₅—, —CH₂—N(Rₐ₇)—CH₂-piperidinylene-(CH₂)₆—, —CH₂—N(Rₐ₇)—CH₂-piperidinylene-(CH₂)₇—, —CH₂—N(Rₐ₇)—CH₂-piperidinylene-(CH₂)₈—, —CH₂—N(Rₐ₇)—(CH₂)₂-piperidinylene-CH₂—, —CH₂—N(Rₐ₇)—(CH₂)₂-piperidinylene-(CH₂)₂—, —CH₂—N(Rₐ₇)—(CH₂)₂-piperidinylene-(CH₂)₃—, —CH₂—N(Rₐ₇)—(CH₂)₂-piperidinylene-(CH₂)₄—, —CH₂—N(Rₐ₇)—(CH₂)₂-piperidinylene-(CH₂)₅—, —CH₂—N(Rₐ₇)—(CH₂)₂-piperidinylene-(CH₂)₆—, —CH₂—N(Rₐ₇)—(CH₂)₂-piperidinylene-(CH₂)₇—, —CH₂—N(Rₐ₇)—(CH₂)₂-piperidinylene-(CH₂)₈—, —(CH₂)₂—N(Rₐ₇)—CH₂-piperidinylene-CH₂—, —(CH₂)₂—N(Rₐ₇)—CH₂-piperidinylene-(CH₂)₂—, —(CH₂)₂—N(Rₐ₇)—CH₂-piperidinylene-(CH₂)₃—, —(CH₂)₂—N(Rₐ₇)—CH₂-piperidinylene-(CH₂)₄—, —(CH₂)₂—N(Rₐ₇)—CH₂-piperidinylene-(CH₂)₅—, —(CH₂)₂—N(Rₐ₇)—CH₂-piperidinylene-(CH₂)₆—, —(CH₂)₂—N(Rₐ₇)—CH₂-piperidinylene-(CH₂)₇—, —(CH₂)₂—N(Rₐ₇)—CH₂-piperidinylene-(CH₂)₈—, —(CH₂)₃—N(Rₐ₇)—CH₂-piperidinylene-CH₂—, —(CH₂)₃—N(Rₐ₇)—CH₂-piperidinylene-(CH₂)₂—, —(CH₂)₃—N(Rₐ₇)—CH₂-piperidinylene-(CH₂)₃—, —(CH₂)₃—N(Rₐ₇)—CH₂-piperidinylene-(CH₂)₈—, —(CH₂)₄—N(Rₐ₇)—CH₂-piperidinylene-CH₂—, (CH₂)₄—N(Rₐ₇)—CH₂-piperidinylene-(CH₂)₂—, —(CH₂)₄—N(Rₐ₇)—CH₂-piperidinylene-(CH₂)₃—, —(CH₂)₄—N(Rₐ₇)—CH₂-piperidinylene-(CH₂)₈—, —(CH₂)₅—N(Rₐ₇)—CH₂-piperidinylene-(CH₂)₃—, —(CH₂)₅—N(Rₐ₇)—CH₂-piperidinylene-(CH₂)₈—, —(CH₂)₆—N(Rₐ₇)—CH₂-piperidinylene-(CH₂)₃—, —(CH₂)₆—N(Rₐ₇)—CH₂-piperidinylene-(CH₂)₈—, —(CH₂)₇—N(Rₐ₇)—CH₂-piperidinylene-(CH₂)₃—, —(CH₂)₈—N(Rₐ₇)—CH₂-piperidinylene-CH₂—, —(CH₂)₈—N(Rₐ₇)—CH₂-piperidinylene-(CH₂)₂—, —(CH₂)₈—N(Rₐ₇)—CH₂-piperidinylene-(CH₂)₃—, —(CH₂)₈—N(Rₐ₇)—CH₂-piperidinylene-(CH₂)₄—, —(CH₂)₈—N(Rₐ₇)—CH₂-piperidinylene-(CH₂)₅—, —(CH₂)₈—N(Rₐ₇)—CH₂-piperidinylene-(CH₂)₆—,-piperazinylene- CH₂—,-piperazinylene-(CH₂)₂—,-piperazinylene-(CH₂)₃—,-piperazinylene-(CH₂)₄—,-piperazinylene-(CH₂)₅—,-piperazinylene-(CH₂)₆—,-piperazinylene-(CH₂)₇—,-piperazinylene-(CH₂)₈—, —CH₂-piperazinylene-CH₂—, —CH₂-piperazinylene-(CH₂)₂—, —CH₂-piperazinylene-(CH₂)₃—, —CH₂-piperazinylene-(CH₂)₄—, —CH₂-piperazinylene-(CH₂)₅—, —CH₂-piperazinylene-(CH₂)₆—, —CH₂-piperazinylene-(CH₂)₇—, —CH₂-piperazinylene-(CH₂)₈—, —(CH₂)₂-piperazinylene-(CH₂)₁—, —(CH₂)₂-piperazinylene-(CH₂)₂—, —(CH₂)₂-piperazinylene-(CH₂)₃—, —(CH₂)₂-piperazinylene-(CH₂)₄—, —(CH₂)₂-piperazinylene-(CH₂)₅—, —(CH₂)₂-piperazinylene-(CH₂)₆—, —(CH₂)₂-piperazinylene-(CH₂)₇—, —(CH₂)₂-piperazinylene-(CH₂)₈—, —(CH₂)₃-piperazinylene-CH₂—, —(CH₂)₃-piperazinylene-(CH₂)₂—, —(CH₂)₃-piperazinylene-(CH₂)₃—, —(CH₂)₃-piperazinylene-(CH₂)₄—, —(CH₂)₃-piperazinylene-(CH₂)₅—, —(CH₂)₃-piperazinylene-(CH₂)₆—, —(CH₂)₃-piperazinylene-(CH₂)₇—, —(CH₂)₃-piperazinylene-(CH₂)₈—, —(CH₂)₄-piperazinylene-CH₂—, piperazinylene-(CH₂)₃—, —(CH₂)₄-piperazinylene-(CH₂)₄—, piperazinylene-(CH₂)₆—, —(CH₂)₄-piperazinylene-(CH₂)₇—, piperazinylene-(CH₂)₁—, —(CH₂)₅-piperazinylene-(CH₂)₂—, piperazinylene-(CH₂)₄—, —(CH₂)₅-piperazinylene-(CH₂)₅—, piperazinylene-(CH₂)₇—, —(CH₂)₅-piperazinylene-(CH₂)₈—, piperazinylene-(CH₂)₂—, —(CH₂)₆-piperazinylene-(CH₂)₃—, (CH₂)₄-piperazinylene-(CH₂)₂—, —(CH₂)₄—(CH₂)₄-piperazinylene-(CH₂)₅—, —(CH₂)₄—(CH₂)₄-piperazinylene-(CH₂)₈—, —(CH₂)₅—(CH₂)₅-piperazinylene-(CH₂)₃—, —(CH₂)₅—(CH₂)₅-piperazinylene-(CH₂)₆—, —(CH₂)₅—(CH₂)₆-piperazinylene-(CH₂)₁—, —(CH₂)₆—(CH₂)₆-piperazinylene-(CH₂)₄—, —(CH₂)₆-piperazinylene-(CH₂)₅—, —(CH₂)₆-piperazinylene-(CH₂)₆—, —(CH₂)₆-piperazinylene-(CH₂)₇—, —(CH₂)₆-piperazinylene-(CH₂)₈—, —(CH₂)₇-piperazinylene-(CH₂)₁—, —(CH₂)₇-piperazinylene-(CH₂)₂—, —(CH₂)₇-piperazinylene-(CH₂)₃—, —(CH₂)₇-piperazinylene-(CH₂)₄—, —(CH₂)₈-piperazinylene-CH₂—, —(CH₂)₈-piperazinylene-(CH₂)₂—, —(CH₂)₈-piperazinylene-(CH₂)₃—, —(CH₂)₈-piperazinylene-(CH₂)₄—, —(CH₂)₈-piperazinylene-(CH₂)₅—, or —(CH₂)₈-piperazinylene-(CH₂)₆—;
wherein each Rₐ₇ independently represents H or C₁₋₃ alkyl;
each phenylene is independently optionally substituted with a substituent selected from the group consisting of deuterium, hydroxy, amino, mercapto, nitro, halogen, cyano, C₁₋₆ alkyl, halogenated C₁₋₆ alkyl, deuterated C₁-6 alkyl, C₁₋₆ alkoxy, halogenated C₁₋₆ alkoxy, C₃₋₆ cycloalkyl, halogenated C₃₋₆ cycloalkyl, deuterated C₃₋₆ cycloalkyl, 4-to 10-membered heterocyclyl, and any combination thereof, and
the piperazinylene and piperidinylene are each independently optionally substituted with a substituent selected from the group consisting of deuterium, hydroxy, amino, mercapto, nitro, halogen, cyano, oxo, C₁₋₆ alkyl, halogenated C₁₋₆ alkyl, deuterated C₁₋₆ alkyl, C₁₋₆ alkoxy, halogenated C₁₋₆ alkoxy, C₃₋₆ cycloalkyl, halogenated C₃₋₆ cycloalkyl, deuterated C₃₋₆ cycloalkyl, 4-to 10-membered heterocyclyl, and any combination thereof. In the present disclosure, unless otherwise specified, any one end of the above-mentioned groups may be connected to R_{w} of the compound of Formula (II), while the other end is connected to R_{w3}. For example, in the group-O—CH₂—, the O can be connected to R_{w} of the compound of Formula (II), and the CH₂ can be connected to R_{w3} of the compound of Formula (II), and vice versa.

In some embodiments, the compound of Formula (II) is also represented by Formula (II-A), Formula (II-B), Formula (II-C), or Formula (II-D):

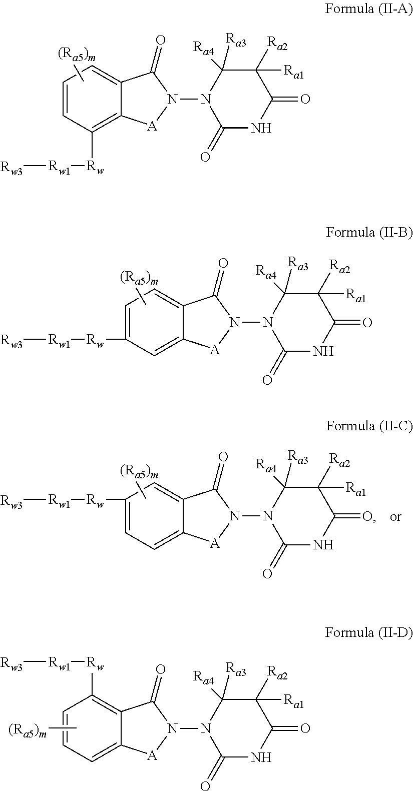

wherein A, (Rₐ₅)ₘ, Rₐ₁, Rₐ₂, Rₐ₃, Rₐ₄, R_{w}, R_{w1}, and R_{w3} are as defined in the compound of Formula (II) above and embodiments thereof.

In some embodiments, the compound of Formula (II) is also represented by Formula (II-1):

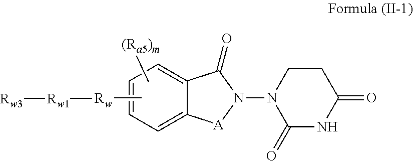

wherein A, (Rₐ₅)ₘ, R_{w}, R_{w1}, and R_{w3} are as defined in the compound of Formula (II) above and embodiments thereof.

In some embodiments, R_{w} of the compound of Formula (II-1) represents O, S, S(O), S(O)₂, alkenylene, alkynylene, or N(Rₐ₆), wherein Rₐ₆ represents H or C₁₋₃ alkyl. In some embodiments, R_{w} of the compound of Formula (II-1) represents a bond.

In some embodiments, the compound of Formula (II) is also represented by Formula (II-2):

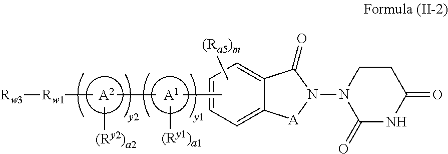

wherein A, (Rₐ₅)ₘ, ring A¹, ring A², y1, (R^{y1})ₐ₁, y2, (R^{y2})ₐ2, R_{w1}, and R_{w3} are as defined in the compound of Formula (II) above and embodiments thereof.

In some embodiments, the compounds of Formula (II) of the present invention and their salts (including pharmaceutically acceptable salts, such as hydrochloride, etc.), prodrugs, solvates, isotopically enriched analogs, polymorphs, stereoisomers (including enantiomers and diastereomers), or mixture of stereoisomers thereof in Table 2 below are provided.

TABLE 2
The compounds of the present disclosure

| Compound | | |
|---|---|---|
| No. | Structure of compound | Compound's name |
| GT-07014 | 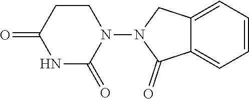 [SMILES: O=C1CCN(N2Cc3ccccc3C2=O)C(=O)N1] | 1-(1-oxoisoindolin-2-yl)dihydropyrimidine- 2,4(1H, 3H)-dione |
| GT-05575 | 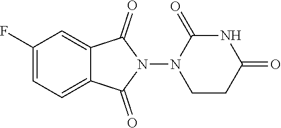 [SMILES: O=C1CCN(N2C(=O)c3ccc(F)cc3C2=O)C(=O)N1] | 2-(2,4-dioxotetrahydropyrimidin-1(2H)-yl)-5- fluoroisoindoline-1,3-dione |
| GT-05576 | 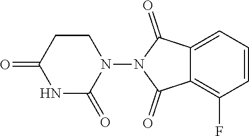 [SMILES: O=C1CCN(N2C(=O)c3cccc(F)c3C2=O)C(=O)N1] | 2-(2,4-dioxotetrahydropyrimidin-1(2H)-yl)-4- fluoroisoindoline-1,3-dione |
| | 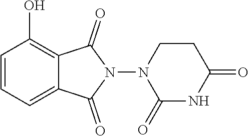 [SMILES: O=C1CCN(N2C(=O)c3cccc(O)c3C2=O)C(=O)N1] | 2-(2,4-dioxotetrahydropyrimidin-1(2H)-yl)-4- hydroxyisoindoline-1,3-dione |
| | 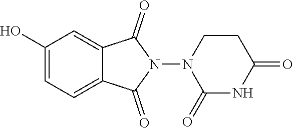 [SMILES: O=C1CCN(N2C(=O)c3ccc(O)cc3C2=O)C(=O)N1] | 2-(2,4-dioxotetrahydropyrimidin-1(2H)-yl)-5- hydroxyisoindoline-1,3-dione |
| GT-04511 | 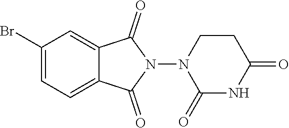 [SMILES: O=C1CCN(N2C(=O)c3ccc(Br)cc3C2=O)C(=O)N1] | 5-bromo-2-(2,4-dioxotetrahydropyrimidin-1(2H)- yl)isoindoline-1,3-dione |
| | 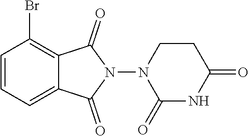 [SMILES: O=C1CCN(N2C(=O)c3cccc(Br)c3C2=O)C(=O)N1] | 4-bromo-2-(2,4-dioxotetrahydropyrimidin-1(2H)- yl)isoindoline-1,3-dione |
| GT-04536 | 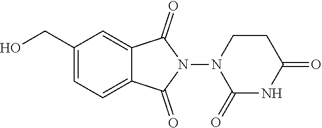 [SMILES: O=C1CCN(N2C(=O)c3ccc(CO)cc3C2=O)C(=O)N1] | 2-(2,4-dioxotetrahydropyrimidin-1(2H)-yl)-5- (hydroxymethyl)isoindoline-1,3-dione |
| | 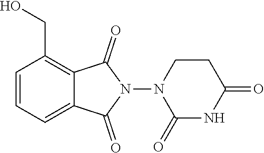 [SMILES: O=C1CCN(N2C(=O)c3cccc(CO)c3C2=O)C(=O)N1] | 2-(2,4-dioxotetrahydropyrimidin-1(2H)-yl)-4- (hydroxymethyl)isoindoline-1,3-dione |
| GT-07968 | 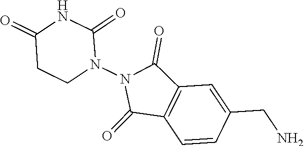 [SMILES: NCc1ccc2c(c1)C(=O)N(N1CCC(=O)NC1=O)C2=O] | 5-(aminomethyl)-2-(2,4-dioxotetrahydropyrimidin- 1(2H)-yl)isoindoline-1,3-dione |
| | 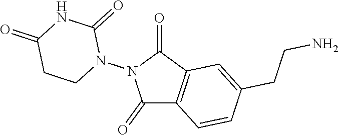 [SMILES: NCCc1ccc2c(c1)C(=O)N(N1CCC(=O)NC1=O)C2=O] | 5-(2-aminoethyl)-2-(2,4- dioxotetrahydropyrimidin-1(2H)-yl)isoindoline- 1,3-dione |
| | 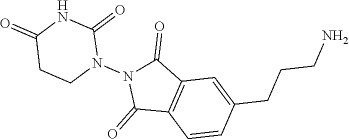 [SMILES: NCCCc1ccc2c(c1)C(=O)N(N1CCC(=O)NC1=O)C2=O] | 5-(3-aminopropyl)-2-(2,4- dioxotetrahydropyrimidin-1(2H)-yl)isoindoline- 1,3-dione |
| | 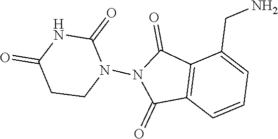 [SMILES: NCc1cccc2c1C(=O)N(N1CCC(=O)NC1=O)C2=O] | 4-(aminomethyl)-2-(2,4-dioxotetrahydropyrimidin- 1(2H)-yl)isoindoline-1,3-dione |
| | 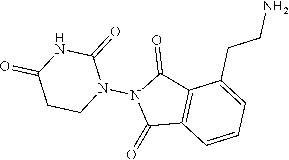 [SMILES: NCCc1cccc2c1C(=O)N(N1CCC(=O)NC1=O)C2=O] | 4-(2-aminoethyl)-2-(2,4- dioxotetrahydropyrimidin-1(2H)-yl)isoindoline- 1,3-dione |
| | 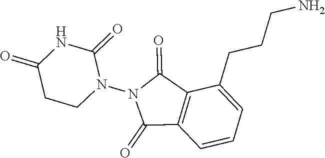 [SMILES: NCCCc1cccc2c1C(=O)N(N1CCC(=O)NC1=O)C2=O] | 4-(3-aminopropyl)-2-(2,4- dioxotetrahydropyrimidin-1(2H)-yl)isoindoline- 1,3-dione |
| | 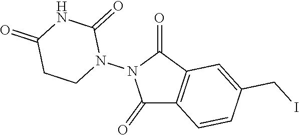 [SMILES: O=C1CCN(N2C(=O)c3ccc(CI)cc3C2=O)C(=O)N1] | 2-(2,4-dioxotetrahydropyrimidin-1(2H)-yl)-5- (iodomethyl)isoindoline-1,3-dione |
| | 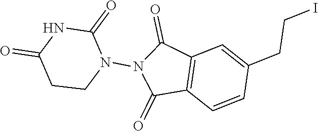 [SMILES: O=C1CCN(N2C(=O)c3ccc(CCI)cc3C2=O)C(=O)N1] | 2-(2,4-dioxotetrahydropyrimidin-1(2H)-yl)-5-(2- iodoethyl)isoindoline-1,3-dione |
| | 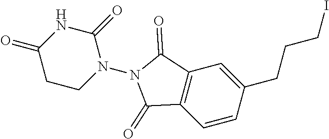 [SMILES: O=C1CCN(N2C(=O)c3ccc(CCCI)cc3C2=O)C(=O)N1] | 2-(2,4-dioxotetrahydropyrimidin-1(2H)-yl)-5-(3- iodopropyl)isoindoline-1,3-dione |
| | 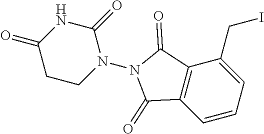 [SMILES: O=C1CCN(N2C(=O)c3cccc(CI)c3C2=O)C(=O)N1] | 2-(2,4-dioxotetrahydropyrimidin-1(2H)-yl)-4- (iodomethyl)isoindoline-1,3-dione |
| | 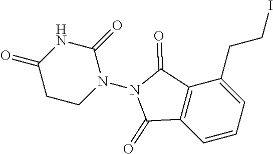 [SMILES: O=C1CCN(N2C(=O)c3cccc(CCI)c3C2=O)C(=O)N1] | 2-(2,4-dioxotetrahydropyrimidin-1(2H)-yl)-4-(2- iodoethyl)isoindoline-1,3-dione |
| | 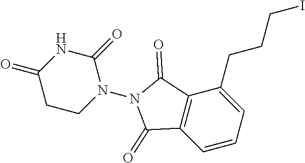 [SMILES: O=C1CCN(N2C(=O)c3cccc(CCCI)c3C2=O)C(=O)N1] | 2-(2,4-dioxotetrahydropyrimidin-1(2H)-yl)-4-(3- iodopropyl)isoindoline-1,3-dione |
| GT-06979 | 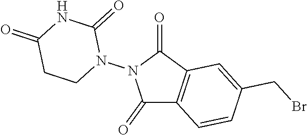 [SMILES: O=C1CCN(N2C(=O)c3ccc(CBr)cc3C2=O)C(=O)N1] | 5-(bromomethyl)-2-(2,4- dioxotetrahydropyrimidin-1(2H)-yl)isoindoline- 1,3-dione |
| | 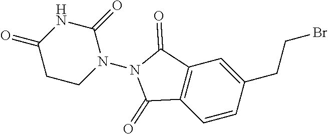 [SMILES: O=C1CCN(N2C(=O)c3ccc(CCBr)cc3C2=O)C(=O)N1] | 5-(2-bromoethyl)-2-(2,4- dioxotetrahydropyrimidin-1(2H)-yl)isoindoline- 1,3-dione |
| | 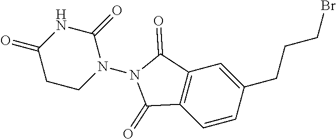 [SMILES: O=C1CCN(N2C(=O)c3ccc(CCCBr)cc3C2=O)C(=O)N1] | 5-(3-bromopropyl)-2-(2,4- dioxotetrahydropyrimidin-1(2H)-yl)isoindoline- 1,3-dione |
| | 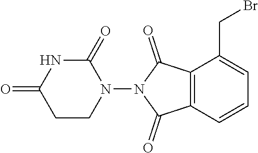 [SMILES: O=C1CCN(N2C(=O)c3cccc(CBr)c3C2=O)C(=O)N1] | 4-(bromomethyl)-2-(2,4- dioxotetrahydropyrimidin-1(2H)-yl)isoindoline- 1,3-dione |
| | 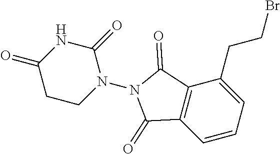 [SMILES: O=C1CCN(N2C(=O)c3cccc(CCBr)c3C2=O)C(=O)N1] | 4-(2-bromoethyl)-2-(2,4- dioxotetrahydropyrimidin-1(2H)-yl)isoindoline- 1,3-dione |
| | 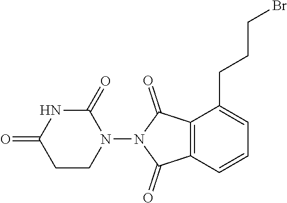 [SMILES: O=C1CCN(N2C(=O)c3cccc(CCCBr)c3C2=O)C(=O)N1] | 4-(3-bromopropyl)-2-(2,4- dioxotetrahydropyrimidin-1(2H)-yl)isoindoline- 1,3-dione |
| | 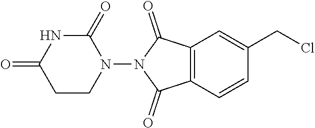 [SMILES: O=C1CCN(N2C(=O)c3ccc(CCl)cc3C2=O)C(=O)N1] | 5-(chloromethyl)-2-(2,4- dioxotetrahydropyrimidin-1(2H)-yl)isoindoline- 1,3-dione |
| | 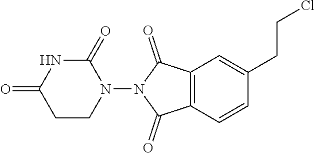 [SMILES: O=C1CCN(N2C(=O)c3ccc(CCCl)cc3C2=O)C(=O)N1] | 5-(2-chloroethyl)-2-(2,4- dioxotetrahydropyrimidin-1(2H)-yl)isoindoline- 1,3-dione |
| | 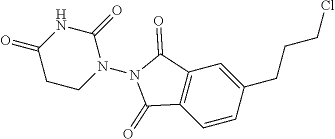 [SMILES: O=C1CCN(N2C(=O)c3ccc(CCCCl)cc3C2=O)C(=O)N1] | 5-(3-chloropropyl)-2-(2,4- dioxotetrahydropyrimidin-1(2H)-yl)isoindoline- 1,3-dione |
| | 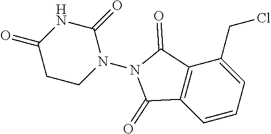 [SMILES: O=C1CCN(N2C(=O)c3cccc(CCl)c3C2=O)C(=O)N1] | 4-(chloromethyl)-2-(2,4- dioxotetrahydropyrimidin-1(2H)-yl)isoindoline- 1,3-dione |
| | 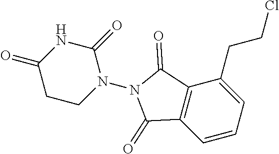 [SMILES: O=C1CCN(N2C(=O)c3cccc(CCCl)c3C2=O)C(=O)N1] | 4-(2-chloroethyl)-2-(2,4- dioxotetrahydropyrimidin-1(2H)-yl)isoindoline- 1,3-dione |
| | 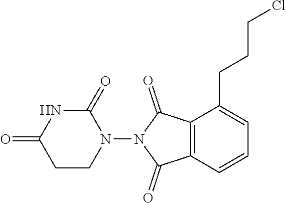 [SMILES: O=C1CCN(N2C(=O)c3cccc(CCCCl)c3C2=O)C(=O)N1] | 4-(3-chloropropyl)-2-(2,4- dioxotetrahydropyrimidin-1(2H)-yl)isoindoline- 1,3-dione |
| | 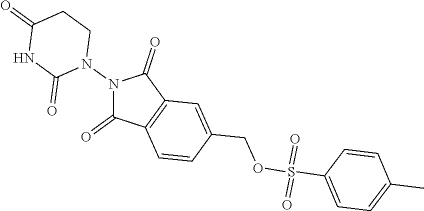 [SMILES: Cc1ccc(S(=O)(=O)OCc2ccc3c(c2)C(=O)N(N2CCC(=O)NC2=O)C3=O)cc1] | (2-(2,4-dioxotetrahydropyrimidin-1(2H)-yl)-1,3- dioxoisoindolin-5-yl)methyl 4- methylbenzenesulfonate |
| | 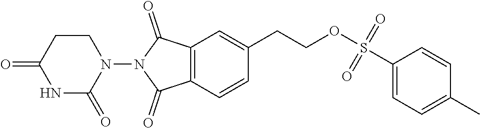 [SMILES: Cc1ccc(S(=O)(=O)OCCc2ccc3c(c2)C(=O)N(N2CCC(=O)NC2=O)C3=O)cc1] | 2-(2-(2,4-dioxotetrahydropyrimidin-1(2H)-yl)-1,3- dioxoisoindolin-5-yl)ethyl 4- methylbenzenesulfonate |
| | 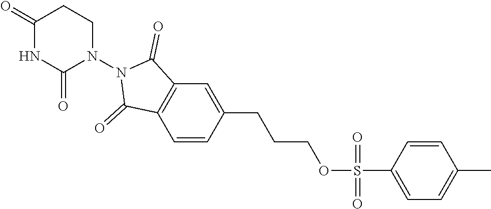 [SMILES: Cc1ccc(S(=O)(=O)OCCCc2ccc3c(c2)C(=O)N(N2CCC(=O)NC2=O)C3=O)cc1] | 3-(2-(2,4-dioxotetrahydropyrimidin-1(2H)-yl)-1,3- dioxoisoindolin-5-yl)propyl 4- methylbenzenesulfonate |
| | 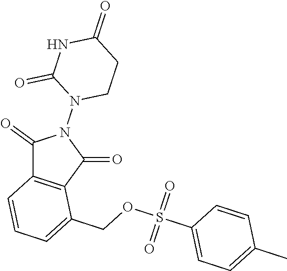 [SMILES: Cc1ccc(S(=O)(=O)OCc2cccc3c2C(=O)N(N2CCC(=O)NC2=O)C3=O)cc1] | (2-(2,4-dioxotetrahydropyrimidin-1(2H)-yl)-1,3- dioxoisoindolin-4-yl)methyl 4- methylbenzenesulfonate |
| | 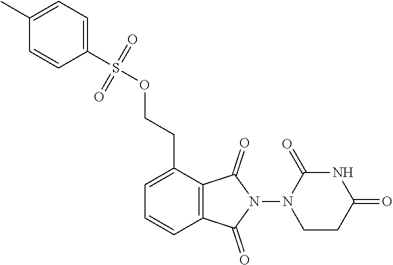 [SMILES: Cc1ccc(S(=O)(=O)OCCc2cccc3c2C(=O)N(N2CCC(=O)NC2=O)C3=O)cc1] | 2-(2-(2,4-dioxotetrahydropyrimidin-1(2H)-yl)-1,3- dioxoisoindolin-4-yl)ethyl 4- methylbenzenesulfonate |
| | 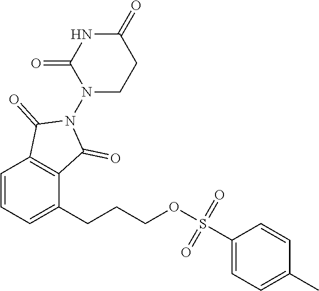 [SMILES: Cc1ccc(S(=O)(=O)OCCCc2cccc3c2C(=O)N(N2CCC(=O)NC2=O)C3=O)cc1] | 3-(2-(2,4-dioxotetrahydropyrimidin-1(2H)-yl)-1,3- dioxoisoindolin-4-yl)propyl 4- methylbenzenesulfonate |
| GT-05591 | 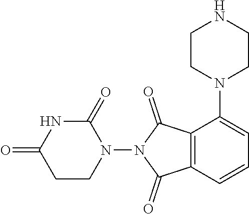 [SMILES: O=C1CCN(N2C(=O)c3cccc(N4CCNCC4)c3C2=O)C(=O)N1] | 2-(2,4-dioxotetrahydropyrimidin-1(2H)-yl)-4- (piperazin-1-yl)isoindoline-1,3-dione |
| GT-05581 | 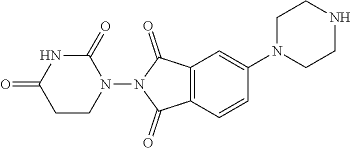 [SMILES: O=C1CCN(N2C(=O)c3ccc(N4CCNCC4)cc3C2=O)C(=O)N1] | 2-(2,4-dioxotetrahydropyrimidin-1(2H)-yl)-5- (piperazin-1-yl)isoindoline-1,3-dione |
| GT-05592 | 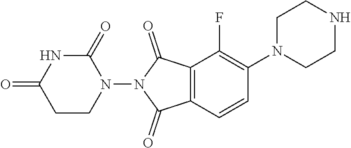 [SMILES: O=C1CCN(N2C(=O)c3ccc(N4CCNCC4)c(F)c3C2=O)C(=O)N1] | 2-(2,4-dioxotetrahydropyrimidin-1(2H)-yl)-4- fluoro-5-(piperazin-1-yl)isoindoline-1,3-dione |
| GT-05584 | 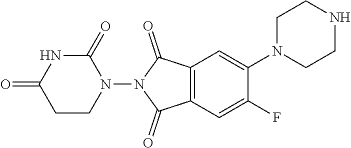 [SMILES: O=C1CCN(N2C(=O)c3cc(F)c(N4CCNCC4)cc3C2=O)C(=O)N1] | 2-(2,4-dioxotetrahydropyrimidin-1(2H)-yl)-5- fluoro-6-(piperazin-1-yl)isoindoline-1,3-dione |
| GT-05593 | 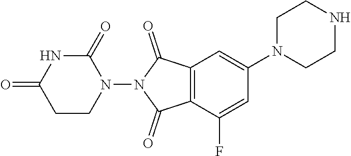 [SMILES: O=C1CCN(N2C(=O)c3cc(N4CCNCC4)cc(F)c3C2=O)C(=O)N1] | 2-(2,4-dioxotetrahydropyrimidin-1(2H)-yl)-4- fluoro-6-(piperazin-1-yl)isoindoline-1,3-dione |
| GT-05587 | 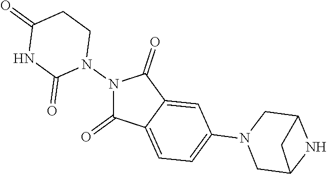 [SMILES: O=C1CCN(N2C(=O)c3ccc(N4CC5CC(C4)N5)cc3C2=O)C(=O)N1] | 5-(3,6-diazabicyclo[3.1.1]heptan-3-yl)-2-(2,4- dioxotetrahydropyrimidin-1(2H)-yl)isoindoline- 1,3-dione |
| GT-05588 | 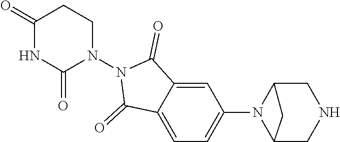 [SMILES: O=C1CCN(N2C(=O)c3ccc(N4C5CNCC4C5)cc3C2=O)C(=O)N1] | 5-(3,6-diazabicyclo[3.1.1 ]heptan-6-yl)-2-(2,4- dioxotetrahydropyrimidin-1(2H)-yl)isoindoline- 1,3-dione |
| GT-05583 | 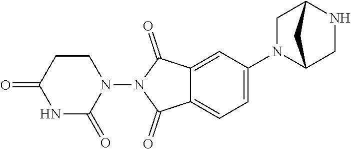 [SMILES: O=C1CCN(N2C(=O)c3ccc(N4C[C@H]5C[C@@H]4CN5)cc3C2=O)C(=O)N1] | 5-((1R, 4R)-2,5-diazabicyclo[2.2.1]heptan-2-yl)-2- (2,4-dioxotetrahydropyrimidin-1(2H)- yl)isoindoline-1,3-dione |
| GT-05582 | 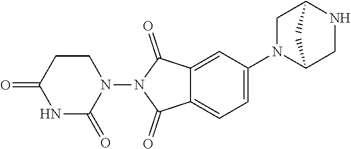 [SMILES: O=C1CCN(N2C(=O)c3ccc(N4C[C@@H]5C[C@H]4CN5)cc3C2=O)C(=O)N1] | 5-((1S, 4S)-2,5-diazabicyclo[2.2.1]heptan-2-yl)-2- (2,4-dioxotetrahydropyrimidin-1(2H)- yl)isoindoline-1,3-dione |
| GT-05589 | 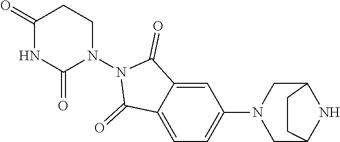 [SMILES: O=C1CCN(N2C(=O)c3ccc(N4CC5CCC(C4)N5)cc3C2=O)C(=O)N1] | 5-(3,8-diazabicyclo[3.2.1]octan-3-yl)-2-(2,4- dioxotetrahydropyrimidin-1(2H)-yl)isoindoline- 1,3-dione |
| GT-05586 | 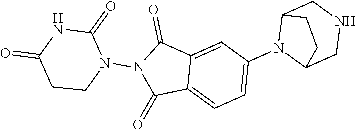 [SMILES: O=C1CCN(N2C(=O)c3ccc(N4C5CCC4CNC5)cc3C2=O)C(=O)N1] | 5-(3,8-diazabicyclo[3.2.1]octan-8-yl)-2-(2,4- dioxotetrahydropyrimidin-1(2H)-yl)isoindoline- 1,3-dione |
| GT-05585 | 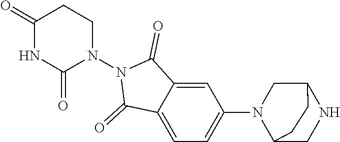 [SMILES: O=C1CCN(N2C(=O)c3ccc(N4CC5CCC4CN5)cc3C2=O)C(=O)N1] | 5-(2,5-diazabicyclo[2.2.2]octan-2-yl)-2-(2,4- dioxotetrahydropyrimidin-1(2H)-yl)isoindoline- 1,3-dione |
| GT-05594 | 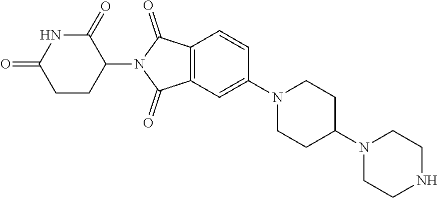 [SMILES: O=C1CCC(N2C(=O)c3ccc(N4CCC(N5CCNCC5)CC4)cc3C2=O)C(=O)N1] | 2-(2,4-dioxotetrahydropyrimidin-1(2H)-yl)-5-(4- (piperazin-1-yl)piperidin-1-yl)isoindoline-1,3- dione |
| GT-04990 | 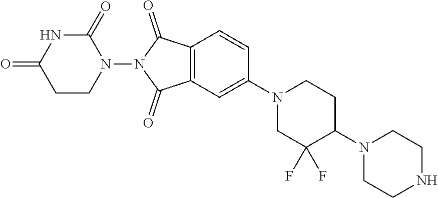 [SMILES: O=C1CCN(N2C(=O)c3ccc(N4CCC(N5CCNCC5)C(F)(F)C4)cc3C2=O)C(=O)N1] | 5-(3,3-difluoro-4-(piperazin-1-yl)piperidin-1-yl)-2- (2,4-dioxotetrahydropyrimidin-1(2H)- yl)isoindoline-1,3-dione |
| GT-05590 | 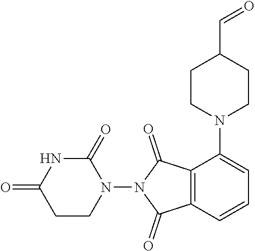 [SMILES: O=CC1CCN(c2cccc3c2C(=O)N(N2CCC(=O)NC2=O)C3=O)CC1] | 1-(2-(2,4-dioxotetrahydropyrimidin-1(2H)-yl)-1,3- dioxoisoindolin-4-yl)piperidine-4-carbaldehyde |
| GT-05580 | 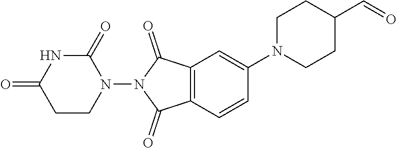 [SMILES: O=CC1CCN(c2ccc3c(c2)C(=O)N(N2CCC(=O)NC2=O)C3=O)CC1] | 1-(2-(2,4-dioxotetrahydropyrimidin-1(2H)-yl)-1,3- dioxoisoindolin-5-yl)piperidine-4-carbaldehyde |
| GT-05595 | 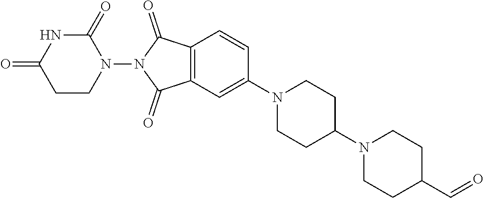 [SMILES: O=CC1CCN(C2CCN(c3ccc4c(c3)C(=O)N(N3CCC(=O)NC3=O)C4=O)CC2)CC1] | 1′-(2-(2,4-dioxotetrahydropyrimidin-1(2H)-yl)-1,3- dioxoisoindolin-5-yl)-[1,4′-bipiperidine]-4- carbaldehyde |
| | 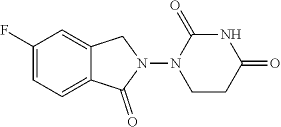 [SMILES: O=C1CCN(N2Cc3cc(F)ccc3C2=O)C(=O)N1] | 1-(5-fluoro-1-oxoisoindolin-2- yl)dihydropyrimidine-2,4(1H, 3H)-dione |
| | 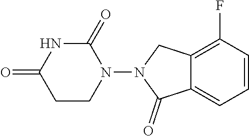 [SMILES: O=C1CCN(N2Cc3c(F)cccc3C2=O)C(=O)N1] | 1-(4-fluoro-1-oxoisoindolin-2- yl)dihydropyrimidine-2,4(1H, 3H)-dione |
| | 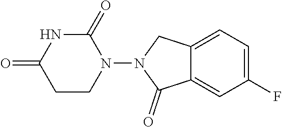 [SMILES: O=C1CCN(N2Cc3ccc(F)cc3C2=O)C(=O)N1] | 1-(6-fluoro-1-oxoisoindolin-2- yl)dihydropyrimidine-2,4(1H, 3H)-dione |
| | 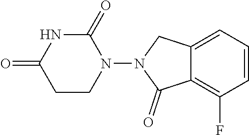 [SMILES: O=C1CCN(N2Cc3cccc(F)c3C2=O)C(=O)N1] | 1-(7-fluoro-1-oxoisoindolin-2- yl)dihydropyrimidine-2,4(1H, 3H)-dione |
| GT-05570 | 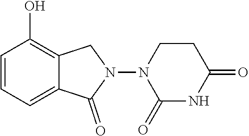 [SMILES: O=C1CCN(N2Cc3c(O)cccc3C2=O)C(=O)N1] | 1-(4-hydroxy-1-oxoisoindolin-2- yl)dihydropyrimidine-2,4(1H, 3H)-dione |
| | 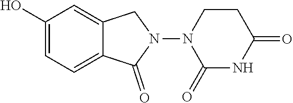 [SMILES: O=C1CCN(N2Cc3cc(O)ccc3C2=O)C(=O)N1] | 1-(5-hydroxy-1-oxoisoindolin-2- yl)dihydropyrimidine-2,4(1H, 3H)-dione |
| | 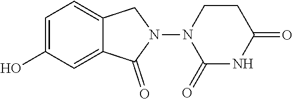 [SMILES: O=C1CCN(N2Cc3ccc(O)cc3C2=O)C(=O)N1] | 1-(6-hydroxy-1-oxoisoindolin-2- yl)dihydropyrimidine-2,4(1H, 3H)-dione |
| | 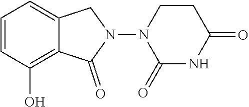 [SMILES: O=C1CCN(N2Cc3cccc(O)c3C2=O)C(=O)N1] | 1-(7-hydroxy-1-oxoisoindolin-2- yl)dihydropyrimidine-2,4(1H, 3H)-dione |
| | 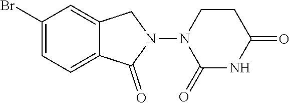 [SMILES: O=C1CCN(N2Cc3cc(Br)ccc3C2=O)C(=O)N1] | 1-(5-bromo-1-oxoisoindolin-2- yl)dihydropyrimidine-2,4(1H, 3H)-dione |
| | 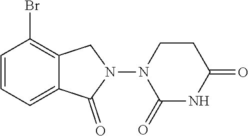 [SMILES: O=C1CCN(N2Cc3c(Br)cccc3C2=O)C(=O)N1] | 1-(4-bromo-1-oxoisoindolin-2- yl)dihydropyrimidine-2,4(1H, 3H)-dione |
| | 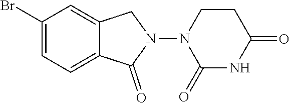 [SMILES: O=C1CCN(N2Cc3cc(Br)ccc3C2=O)C(=O)N1] | 1-(5-bromo-1-oxoisoindolin-2- yl)dihydropyrimidine-2,4(1H, 3H)-dione |
| | 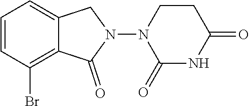 [SMILES: O=C1CCN(N2Cc3cccc(Br)c3C2=O)C(=O)N1] | 1-(7-bromo-1-oxoisoindolin-2- yl)dihydropyrimidine-2,4(1H, 3H)-dione |
| GT-05567 | 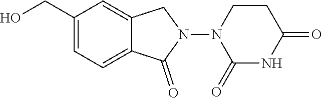 [SMILES: O=C1CCN(N2Cc3cc(CO)ccc3C2=O)C(=O)N1] | 1-(5-(hydroxymethyl)-1-oxoisoindolin-2- yl)dihydropyrimidine-2,4(1H, 3H)-dione |
| | 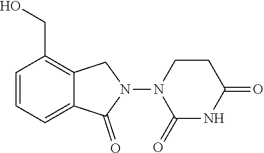 [SMILES: O=C1CCN(N2Cc3c(CO)cccc3C2=O)C(=O)N1] | 1-(4-(hydroxymethyl)-1-oxoisoindolin-2- yl)dihydropyrimidine-2,4(1H, 3H)-dione |
| GT-08118 | 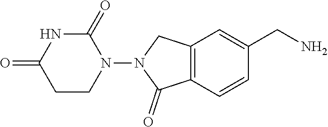 [SMILES: NCc1ccc2c(c1)CN(N1CCC(=O)NC1=O)C2=O] | 1-(5-(aminomethyl)-1-oxoisoindolin-2- yl)dihydropyrimidine-2,4(1H, 3H)-dione |
| | 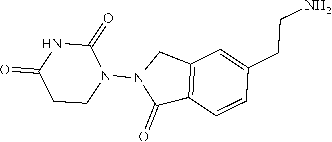 [SMILES: NCCc1ccc2c(c1)CN(N1CCC(=O)NC1=O)C2=O] | 1-(5-(2-aminoethyl)-1-oxoisoindolin-2- yl)dihydropyrimidine-2,4(1H, 3H)-dione |
| | 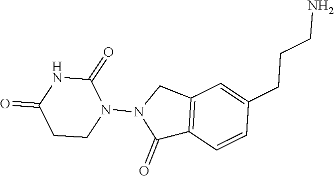 [SMILES: NCCCc1ccc2c(c1)CN(N1CCC(=O)NC1=O)C2=O] | 1-(5-(3-aminopropyl)-1-oxoisoindolin-2- yl)dihydropyrimidine-2,4(1H, 3H)-dione |
| | 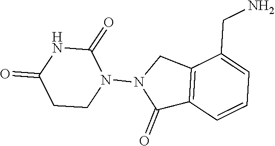 [SMILES: NCc1cccc2c1CN(N1CCC(=O)NC1=O)C2=O] | 1-(4-(aminomethyl)-1-oxoisoindolin-2- yl)dihydropyrimidine-2,4(1H, 3H)-dione |
| | 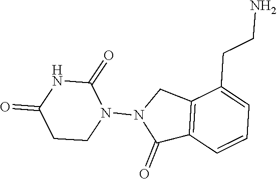 [SMILES: NCCc1cccc2c1CN(N1CCC(=O)NC1=O)C2=O] | 1-(4-(2-aminoethyl)-1-oxoisoindolin-2- yl)dihydropyrimidine-2,4(1H, 3H)-dione |
| | 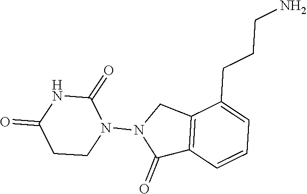 [SMILES: NCCCc1cccc2c1CN(N1CCC(=O)NC1=O)C2=O] | 1-(4-(3-aminopropyl)-1-oxoisoindolin-2- yl)dihydropyrimidine-2,4(1H, 3H)-dione |
| | 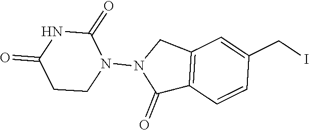 [SMILES: O=C1CCN(N2Cc3cc(CI)ccc3C2=O)C(=O)N1] | 1-(5-(iodomethyl)-1-oxoisoindolin-2- yl)dihydropyrimidine-2,4(1H, 3H)-dione |
| | 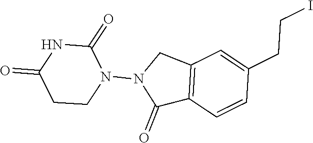 [SMILES: O=C1CCN(N2Cc3cc(CCI)ccc3C2=O)C(=O)N1] | 1-(5-(2-iodoethyl)-1-oxoisoindolin-2- yl)dihydropyrimidine-2,4(1H, 3H)-dione |
| | 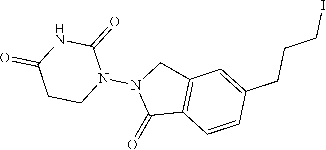 [SMILES: O=C1CCN(N2Cc3cc(CCCI)ccc3C2=O)C(=O)N1] | 1-(5-(3-iodopropyl)-1-oxoisoindolin-2- yl)dihydropyrimidine-2,4(1H, 3H)-dione |
| | 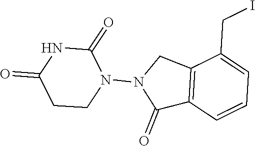 [SMILES: O=C1CCN(N2Cc3c(CI)cccc3C2=O)C(=O)N1] | 1-(4-(iodomethyl)-1-oxoisoindolin-2- yl)dihydropyrimidine-2,4(1H, 3H)-dione |
| | 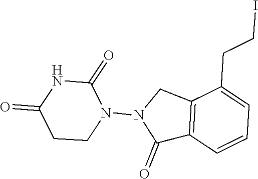 [SMILES: O=C1CCN(N2Cc3c(CCI)cccc3C2=O)C(=O)N1] | 1-(4-(2-iodoethyl)-1-oxoisoindolin-2- yl)dihydropyrimidine-2,4(1H, 3H)-dione |
| | 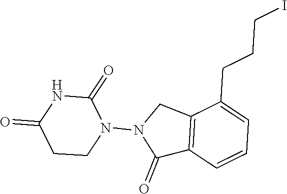 [SMILES: O=C1CCN(N2Cc3c(CCCI)cccc3C2=O)C(=O)N1] | 1-(4-(3-iodopropyl)-1-oxoisoindolin-2- yl)dihydropyrimidine-2,4(1H, 3H)-dione |
| GT-05568 | 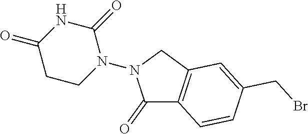 [SMILES: O=C1CCN(N2Cc3cc(CBr)ccc3C2=O)C(=O)N1] | 1-(5-(bromomethyl)-1-oxoisoindolin-2- yl)dihydropyrimidine-2,4(1H, 3H)-dione |
| | 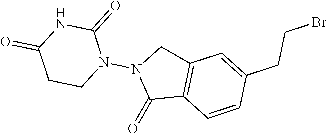 [SMILES: O=C1CCN(N2Cc3cc(CCBr)ccc3C2=O)C(=O)N1] | 1-(5-(2-bromoethyl)-1-oxoisoindolin-2- yl)dihydropyrimidine-2,4(1H, 3H)-dione |
| | 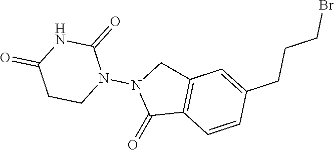 [SMILES: O=C1CCN(N2Cc3cc(CCCBr)ccc3C2=O)C(=O)N1] | 1-(5-(3-bromopropyl)-1-oxoisoindolin-2- yl)dihydropyrimidine-2,4(1H, 3H)-dione |
| | 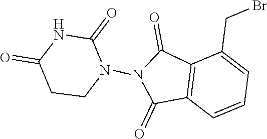 [SMILES: O=C1CCN(N2C(=O)c3cccc(CBr)c3C2=O)C(=O)N1] | 1-(4-(bromomethyl)-1-oxoisoindolin-2- yl)dihydropyrimidine-2,4(1H, 3H)-dione |
| | 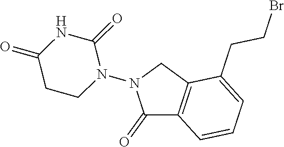 [SMILES: O=C1CCN(N2Cc3c(CCBr)cccc3C2=O)C(=O)N1] | 1-(4-(2-bromoethyl)-1-oxoisoindolin-2- yl)dihydropyrimidine-2,4(1H, 3H)-dione |
| | 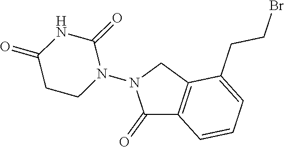 [SMILES: O=C1CCN(N2Cc3c(CCBr)cccc3C2=O)C(=O)N1] | 1-(4-(3-bromopropyl)-1-oxoisoindolin-2- yl)dihydropyrimidine-2,4(1H, 3H)-dione |
| | 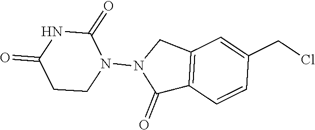 [SMILES: O=C1CCN(N2Cc3cc(CCl)ccc3C2=O)C(=O)N1] | 1-(5-(chloromethyl)-1-oxoisoindolin-2- yl)dihydropyrimidine-2,4(1H, 3H)-dione |
| | 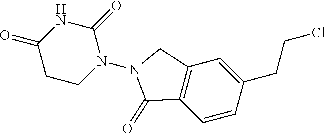 [SMILES: O=C1CCN(N2Cc3cc(CCCl)ccc3C2=O)C(=O)N1] | 1-(5-(2-chloroethyl)-1-oxoisoindolin-2- yl)dihydropyrimidine-2,4(1H, 3H)-dione |
| | 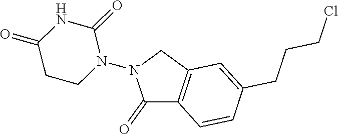 [SMILES: O=C1CCN(N2Cc3cc(CCCCl)ccc3C2=O)C(=O)N1] | 1-(5-(3-chloropropyl)-1-oxoisoindolin-2- yl)dihydropyrimidine-2,4(1H, 3H)-dione |
| | 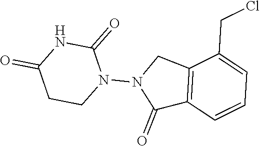 [SMILES: O=C1CCN(N2Cc3c(CCl)cccc3C2=O)C(=O)N1] | 1-(4-(chloromethyl)-1-oxoisoindolin-2- yl)dihydropyrimidine-2,4(1H, 3H)-dione |
| | 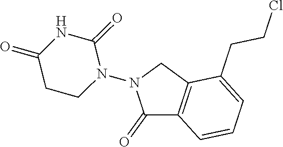 [SMILES: O=C1CCN(N2Cc3c(CCCl)cccc3C2=O)C(=O)N1] | 1-(4-(2-chloroethyl)-1-oxoisoindolin-2- yl)dihydropyrimidine-2,4(1H, 3H)-dione |
| | 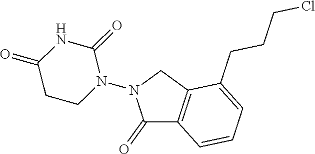 [SMILES: O=C1CCN(N2Cc3c(CCCCl)cccc3C2=O)C(=O)N1] | 1-(4-(3-chloropropyl)-1-oxoisoindolin-2- yl)dihydropyrimidine-2,4(1H, 3H)-dione |
| | 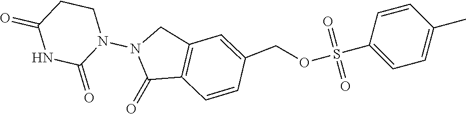 [SMILES: Cc1ccc(S(=O)(=O)OCc2ccc3c(c2)CN(N2CCC(=O)NC2=O)C3=O)cc1] | (2-(2,4-dioxotetrahydropyrimidin-1(2H)-yl)-1- oxoisoindolin-5-yl)methyl 4- methylbenzenesulfonate |
| | 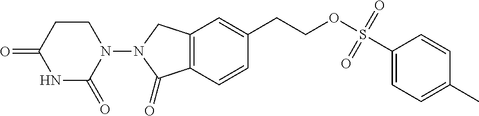 [SMILES: Cc1ccc(S(=O)(=O)OCCc2ccc3c(c2)CN(N2CCC(=O)NC2=O)C3=O)cc1] | 2-(2-(2,4-dioxotetrahydropyrimidin-1(2H)-yl)-1- oxoisoindolin-5-yl)ethyl 4- methylbenzenesulfonate |
| | 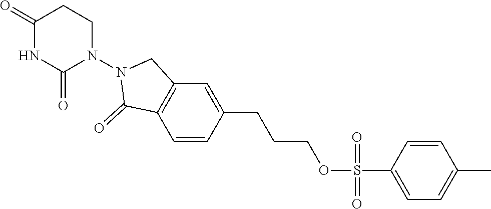 [SMILES: Cc1ccc(S(=O)(=O)OCCCc2ccc3c(c2)CN(N2CCC(=O)NC2=O)C3=O)cc1] | 3-(2-(2,4-dioxotetrahydropyrimidin-1(2H)-yl)-1- oxoisoindolin-5-yl)propyl 4- methylbenzenesulfonate |
| | 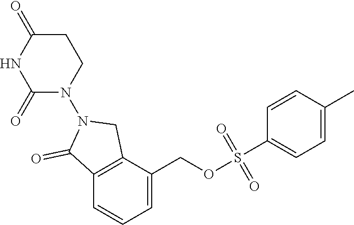 [SMILES: Cc1ccc(S(=O)(=O)OCc2cccc3c2CN(N2CCC(=O)NC2=O)C3=O)cc1] | (2-(2,4-dioxotetrahydropyrimidin-1(2H)-yl)-1- oxoisoindolin-4-yl)methyl 4- methylbenzenesulfonate |
| | 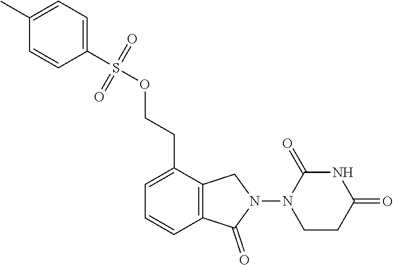 [SMILES: Cc1ccc(S(=O)(=O)OCCc2cccc3c2CN(N2CCC(=O)NC2=O)C3=O)cc1] | 2-(2-(2,4-dioxotetrahydropyrimidin-1(2H)-yl)-1- oxoisoindolin-4-yl)ethyl 4- methylbenzenesulfonate |
| | 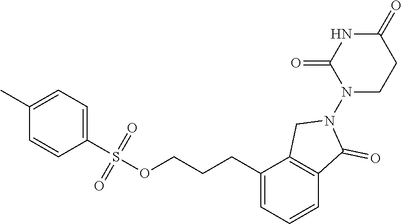 [SMILES: Cc1ccc(S(=O)(=O)OCCCc2cccc3c2CN(N2CCC(=O)NC2=O)C3=O)cc1] | 3-(2-(2,4-dioxotetrahydropyrimidin-1(2H)-yl)-1- oxoisoindolin-4-yl)propyl 4- methylbenzenesulfonate |
| | 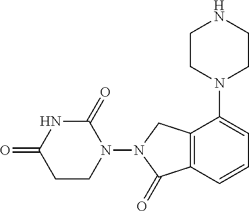 [SMILES: O=C1CCN(N2Cc3c(cccc3N3CCNCC3)C2=O)C(=O)N1] | 1-(1-oxo-4-(piperazin-1-yl)isoindolin-2- yl)dihydropyrimidine-2,4(1H, 3H)-dione |
| | 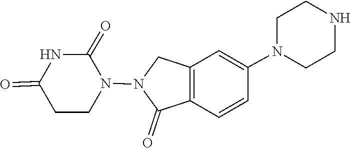 [SMILES: O=C1CCN(N2Cc3cc(N4CCNCC4)ccc3C2=O)C(=O)N1] | 1-(1-oxo-5-(piperazin-1-yl)isoindolin-2- yl)dihydropyrimidine-2,4(1H, 3H)-dione |
| | 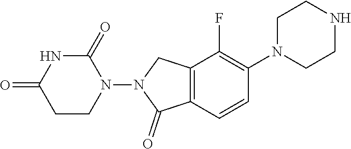 [SMILES: O=C1CCN(N2Cc3c(ccc(N4CCNCC4)c3F)C2=O)C(=O)N1] | 1-(4-fluoro-1-oxo-5-(piperazin-1-yl)isoindolin-2- yl)dihydropyrimidine-2,4(1H, 3H)-dione |
| | 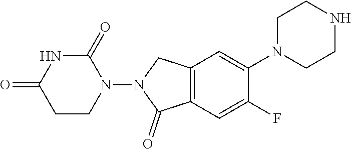 [SMILES: O=C1CCN(N2Cc3cc(N4CCNCC4)c(F)cc3C2=O)C(=O)N1] | 1-(6-fluoro-1-oxo-5-(piperazin-1-yl)isoindolin-2- yl)dihydropyrimidine-2,4(1H, 3H)-dione |
| | 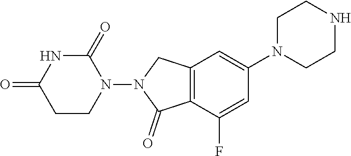 [SMILES: O=C1CCN(N2Cc3cc(N4CCNCC4)cc(F)c3C2=O)C(=O)N1] | 1-(7-fluoro-1-oxo-5-(piperazin-1-yl)isoindolin-2- yl)dihydropyrimidine-2,4(1H, 3H)-dione |
| | 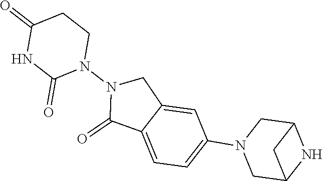 [SMILES: O=C1CCN(N2Cc3cc(N4CC5CC(C4)N5)ccc3C2=O)C(=O)N1] | 1-(5-(3,6-diazabicyclo[3.1.1 ]heptan-3-yl)-1- oxoisoindolin-2-yl)dihydropyrimidine-2,4(1H, 3H)- dione |
| | 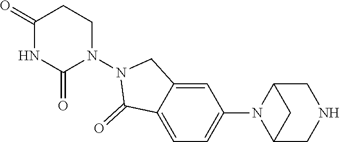 [SMILES: O=C1CCN(N2Cc3cc(N4C5CNCC4C5)ccc3C2=O)C(=O)N1] | 1-(5-(3,6-diazabicyclo[3.1.1]heptan-6-yl)-1- oxoisoindolin-2-yl)dihydropyrimidine-2,4(1H, 3H)- dione |
| | 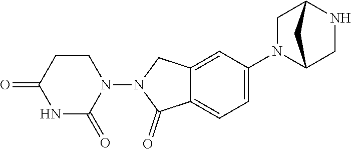 [SMILES: O=C1CCN(N2Cc3cc(N4C[C@H]5C[C@@H]4CN5)ccc3C2=O)C(=O)N1] | 1-(5-((1R, 4R)-2,5-diazabicyclo[2.2.1]heptan-2-yl)- 1-oxoisoindolin-2-yl)dihydropyrimidine- 2,4(1H, 3H)-dione |
| | 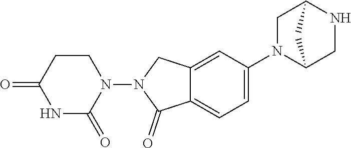 [SMILES: O=C1CCN(N2Cc3cc(N4C[C@@H]5C[C@H]4CN5)ccc3C2=O)C(=O)N1] | 1-(5-((1S, 4S)-2,5-diazabicyclo[2.2.1]heptan-2-yl)- 1-oxoisoindolin-2-yl)dihydropyrimidine- 2,4(1H, 3H)-dione |
| | 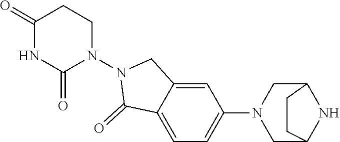 [SMILES: O=C1CCN(N2Cc3cc(N4CC5CCC(C4)N5)ccc3C2=O)C(=O)N1] | 1-(5-(3,8-diazabicyclo[3.2.1]octan-3-yl)-1- oxoisoindolin-2-yl)dihydropyrimidine-2,4(1H, 3H)- dione |
| | 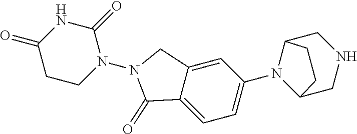 [SMILES: O=C1CCN(N2Cc3cc(N4C5CCC4CNC5)ccc3C2=O)C(=O)N1] | 1-(5-(3,8-diazabicyclo[3.2.1]octan-8-yl)-1- oxoisoindolin-2-yl)dihydropyrimidine-2,4(1H, 3H)- dione |
| | 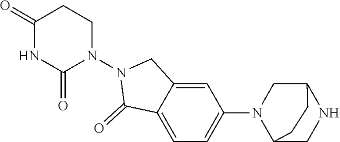 [SMILES: O=C1CCN(N2Cc3cc(N4CC5CCC4CN5)ccc3C2=O)C(=O)N1] | 1-(5-(2,5-diazabicyclo[2.2.2]octan-2-yl)-1- oxoisoindolin-2-yl)dihydropyrimidine-2,4(1H, 3H)- dione |
| | 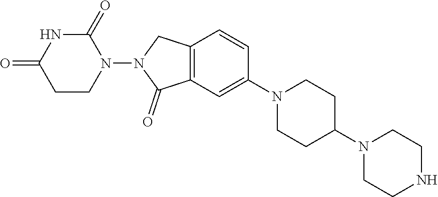 [SMILES: O=C1CCN(N2Cc3ccc(N4CCC(N5CCNCC5)CC4)cc3C2=O)C(=O)N1] | 1-(1-oxo-6-(4-(piperazin-1-yl)piperidin-1- yl)isoindolin-2-yl)dihydropyrimidine-2,4(1H, 3H)- dione |
| | 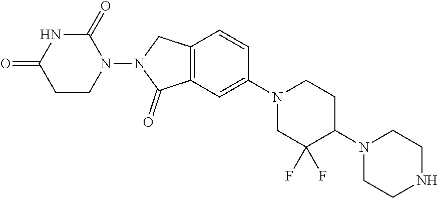 [SMILES: O=C1CCN(N2Cc3ccc(N4CCC(N5CCNCC5)C(F)(F)C4)cc3C2=O)C(=O)N1] | 1-(6-(3,3-difluoro-4-(piperazin-1-yl)piperidin-1- yl)-1-oxoisoindolin-2-yl)dihydropyrimidine- 2,4(1H, 3H)-dione |
| | 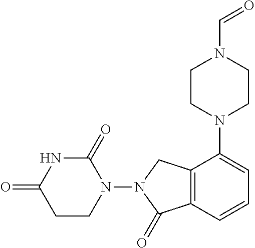 [SMILES: O=CN1CCN(c2cccc3c2CN(N2CCC(=O)NC2=O)C3=O)CC1] | 1-(2-(2,4-dioxotetrahydropyrimidin-1(2H)-yl)-1- oxoisoindolin-4-yl)piperidine-4-carbaldehyde |
| | 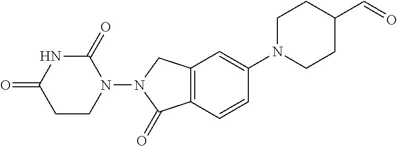 [SMILES: O=CC1CCN(c2ccc3c(c2)CN(N2CCC(=O)NC2=O)C3=O)CC1] | 1-(2-(2,4-dioxotetrahydropyrimidin-1(2H)-yl)-1- oxoisoindolin-5-yl)piperidine-4-carbaldehyde |
| | 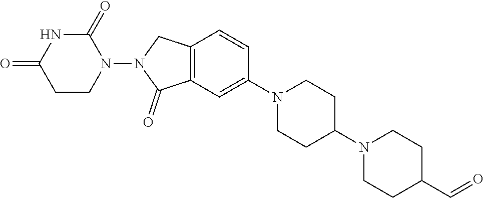 [SMILES: O=CC1CCN(C2CCN(c3ccc4c(c3)C(=O)N(N3CCC(=O)NC3=O)C4)CC2)CC1] | 1′-(2-(2,4-dioxotetrahydropyrimidin-1(2H)-yl)-3- oxoisoindolin-5-yl)-[1,4′-bipiperidine]-4- carbaldehyde |

### II. Other Forms of Compounds (Including Salts, Enantiomers, Stereoisomers, Solvates, Isotopically Enriched Analogs, Prodrugs, or Polymorphs of Compounds)

The compounds of the present disclosure have the structures of any one of Formula (I), Formula (II), Formula (I-1), Formula (I-2), Formula (I-3), Formula (I-4), Formula (I-5-1), Formula (I-5-2), Formula (I-5-3), Formula (I-5-4), Formula (I-6), Formula (I-7), Formula (I-8), Formula (I-9), Formula (I-10-1), Formula (I-10-2), Formula (I-11), Formula (I-12), Formula (I-13-1), Formula (I-13-2), Formula (I-14), Formula (I-15), Formula (I-16), Formula (I-17), Formula (I-18-1), Formula (I-18-2), Formula (I-19), Formula (I-20), Formula (I-21), Formula (I-22), Formula (I-23), Formula (I-24), Formula (I-25), Formula (I-26), Formula (I-27-1), Formula (I-27-2), Formula (I-28), Formula (I-29), Formula (I-30), Formula (I-31), Formula (I-32), or Formula (I-33). Unless otherwise specified, all references to the compounds of the present disclosure also include compounds of any one of Formula (I), Formula (II), Formula (I-1), Formula (I-2), Formula (I-3), Formula (I-4), Formula (I-5-1), Formula (I-5-2), Formula (I-5-3), Formula (I-5-4), Formula (I-6), Formula (I-7), Formula (I-8), Formula (I-9), Formula (I-10-1), Formula (I-10-2), Formula (I-11), Formula (I-12), Formula (I-13-1), Formula (I-13-2), Formula (I-14), Formula (I-15), Formula (I-16), Formula (I-17), Formula (I-18-1), Formula (I-18-2), Formula (I-19), Formula (I-20), Formula (I-21), Formula (I-22), Formula (I-23), Formula (I-24), Formula (I-25), Formula (I-26), Formula (I-27-1), Formula (I-27-2), Formula (I-28), Formula (I-29), Formula (I-30), Formula (I-31), Formula (I-32), or Formula (I-33) and specific compounds within the scope of these general formulae.

It should be recognized that compounds of the present disclosure (including Formula (I), Formula (II), Formula (I-1), Formula (I-2), Formula (I-3), Formula (I-4), Formula (I-5-1), Formula (I-5-2), Formula (I-5-3), Formula (I-5-4), Formula (I-6), Formula (I-7), Formula (I-8), Formula (I-9), Formula (I-10-1), Formula (I-10-2), Formula (I-11), Formula (I-12), Formula (I-13-1), Formula (I-13-2), Formula (I-14), Formula (I-15), Formula (I-16), Formula (I-17), Formula (I-18-1), Formula (I-18-2), Formula (I-19), Formula (I-20), Formula (I-21), Formula (I-22), Formula (I-23), Formula (I-24), Formula (I-25), Formula (I-26), Formula (I-27-1), Formula (I-27-2), Formula (I-28), Formula (I-29), Formula (I-30), Formula (I-31), Formula (I-32), or Formula (I-33) may have a stereo-configuration and thus can exist in more than one stereoisomeric form. The present disclosure also relates to optically enriched compounds having a stereo-configuration, e.g., greater than about 90% enantiomeric/diastereomeric excess (“ee”), such as about 95% ee or 97% ee, or greater than 99% ee, and mixtures thereof, including racemic mixtures. As used herein, “optically enriched” means that a mixture of enantiomers consists of a significantly greater proportion of one enantiomer, and can be described by enantiomeric excess (ee %). Purification of isomers and separation of mixtures of isomers can be accomplished by standard techniques known in the art (e.g., column chromatography, preparative TLC, preparative HPLC, asymmetric synthesis (e.g., by using chiral intermediates) and/or or chiral resolution, etc.).

In some embodiments, polymorph forms or salts of the compounds of the present disclosure are also provided. Salts of the compounds of the present disclosure can be pharmaceutically acceptable salts including, but not limited to, hydrohalides (including hydrochlorides and hydrobromates), sulfate, citrate, maleate, mesylate, lactate, lactobionate, L-tartrate, fumarate, L-malate, α-ketoglutarate, hippurate, D-glucuronate, D-gluconate, α-D-glucoheptonate, glycolate, mucate, L-ascorbate, orotate, picrate, glycinate, alaninate, arginate, cinnamate, laurate, pamoate, sebacate, besylate, methanesulfonate, ethanesulfonate, edisylate, formate, acetate, 2,2-dichloroacetate, trimethylacetate, propionate, valerate, palmitate, triphenylacetate, 2-ethyl-butane-1,4-dioate, iodate, nicotinate, L-pyroglutamate, L-prolinate, ferulate, 2-hydroxyethanesulfonate, nitrate, gentisate, cholate, salicylate, terephthalate, glutarate, adipate, stearate, oleate, undecylenate, camphorate, camsylate, dodecylsulfate, phosphate, thiocyanate, dihydrogen phosphate, pyrophosphate, metaphosphate, oxalate, malonate, benzoate, mandelate, succinate, pyruvate, 4-chlorobenzenesulfonate, 1,5-naphthalenedisulfonate, 3-hydroxy-2-naphthoate, 1-hydroxy-2-naphthoate, 2-naphthalenesulfonate, trifluoroacetate, glycolate, or 4-methylbenzenesulfonate, etc. The compounds of the present disclosure can exist as non-solvated or solvated forms in pharmaceutically acceptable solvents such as water, ethanol, and the like. In some embodiments, the compounds of the present disclosure can be prepared as prodrugs or precursor drugs. Prodrugs can be converted into parent drugs in the body to play their role. In some embodiments, isotopically-labeled compounds of the present disclosure are also provided, examples of which include deuterium (D or 2H).

### III. Compositions/Formulations

In some embodiments, the present disclosure provides a pharmaceutical composition comprising as active ingredient the compound of the present disclosure (the compound of Formula (I), or the compound of Formula (II)) or a pharmaceutically acceptable salt, solvate, isotopically enriched analog, polymorph, prodrug, stereoisomer (including enantiomer), or mixture of stereoisomers thereof, and at least one pharmaceutically acceptable carrier.

In some embodiments, pharmaceutically acceptable carriers include, but are not limited to, fillers, stabilizers, dispersants, suspending agents, diluents, excipients, thickeners, colorants, solvents, or encapsulating materials. Each carrier must be “acceptable” in the sense of being compatible with the other ingredients of the formulation (including the compounds useful in the present disclosure) and not injurious to the patient. Some examples of materials that can be used as pharmaceutically acceptable carriers include: sugars such as lactose, glucose, and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository wax; oils such as peanut oil, cottonseed oil, safflower oil, sesame oil, olive oil, corn oil and soybean oil; glycols such as propylene glycol; polyols such as glycerol, sorbitol, mannitol, and polyethylene glycol; esters such as ethyl oleate and ethyl laurate; agar; buffers such as magnesium hydroxide and aluminum hydroxide; surfactant phosphate buffer solution; polyethylene oxide, polyvinylpyrrolidone, polyacrylamide, poloxamer; and other common non-toxic compatible substances used in pharmaceutical formulations.

The pharmaceutical composition of the present disclosure can further comprise at least one second therapeutic agent, e.g., an anticancer agent. The second therapeutic agent may be used in combination with the compounds of Formula (I)(or the compounds of Formula (II)) described in the present disclosure to treat the diseases or disorders as disclosed herein. The second therapeutic agent includes, but is not limited to, chemotherapeutic agents, immunotherapeutic agents, gene therapy agents, and the like.

The pharmaceutical composition of the present disclosure comprising, as an active ingredient, the compounds of the present disclosure (the compounds of Formula (I) or Formula (II)) or a pharmaceutically acceptable salt thereof can be formulated into any suitable formulations such as sprays, patches, tablets (such as conventional tablets, dispersible tablets, orally disintegrating tablets), capsules (such as soft capsules, hard capsules, enteric-coated capsules), dragees, troches, powders, granules, powder injections, suppositories, or liquid formulations (such as suspensions (e.g., aqueous or oily suspensions), solutions, emulsions, or syrups), or conventional injection dosage forms such as injectable solutions (e.g., sterile injectable solutions formulated according to methods known in the art using water, Ringer's solution, or isotonic sodium chloride solution or the like as a vehicle or solvent) or lyophilized injectable formulation and the like, depending upon a suitable route of administration (including, but not limited to, nasal administration, inhalation administration, topical administration, oral administration, oral mucosal administration, rectal administration, intrapleural administration, intraperitoneal administration, vaginal administration, intramuscular administration, subcutaneous administration, transdermal administration, epidural administration, intrathecal administration, and intravenous administration). Those skilled in the art can also formulate the compounds of the present disclosure (the compounds of Formula (I) or Formula (II)) into conventional, dispersible, chewable, orally disintegrating or rapidly dissolving formulations, or sustained-release capsules or controlled-release capsules as needed.

The compounds of the present disclosure (the compounds of Formula (I) or Formula (II)), as an active ingredient, is contained in the pharmaceutically acceptable carrier or diluent in an amount sufficient to deliver to a subject a therapeutically effective amount for the indication to be treated, without causing serious toxic effects in the subject treated. A dosage of the active compound for all diseases or disorders mentioned herein ranges, for example, from about 5 ng/kg to 500 mg/kg, from about 10 ng/kg to 300 mg/kg per day, such as from 0.1 to 100 mg/kg, or from 0.5 to about 25 mg per kilogram body weight of the subject per day.

The compounds of the present disclosure (the compounds of Formula (I) or Formula (II)) or pharmaceutically acceptable salts thereof may be conveniently administered in any suitable unit dosage form. Suitable unit dosage form specifications include, but are not limited to, less than 1 mg, 1 mg to 3000 mg, 5 mg to 1000 mg, for example, 5 to 500 mg, 25 to 250 mg of active ingredient per unit dosage form.

### IV. Kits/Packaged Products

The compounds of the present disclosure (the compound of Formula (I) or Formula (II)) or a pharmaceutically acceptable salt, solvate, isotopically enriched analog, polymorph, prodrug, stereoisomer (including enantiomer), or mixture of stereoisomers thereof is used as a medicament. The medicament of the present disclosure or the pharmaceutical composition of the present disclosure may be presented in a kit/packaged product. The kit/packaged product may include a package or container including, but not limited to, ampoules, blister packs, pharmaceutical plastic bottles, vials, pharmaceutical glass bottles, containers, syringes, laminated flexible packaging, co-extruded film infusion containers, test tubes and dispensing devices, and the like. The kit/packaged product may contain instructions for use of the product.

### V. Methods and Uses

The compounds of Formula (I) or a pharmaceutically acceptable salt, solvate, isotopically enriched analog, polymorph, prodrug, stereoisomer (including enantiomer), or mixture of stereoisomers thereof can be used as a medicament. Especially the compounds of Formula (I) or a pharmaceutically acceptable salt, solvate, isotopically enriched analog, polymorph, prodrug, stereoisomer (including enantiomer), or mixture of stereoisomers thereof can be used for the manufacture of a medicament for the prevention and/or treatment of diseases or disorders selected from the group consisting of tumors, autoinflammatory diseases, inflammatory diseases, autoimmune diseases, neurological disorders, respiratory diseases, hemorrhagic shock, transplant rejection, multiple organ dysfunction syndrome (MODS), sarcoidosis, adult respiratory distress syndrome, cardiovascular diseases, Richter syndrome (RS), acute liver failure, diabetes, Kennedy disease, seborrheic alopecia, hirsutism, skin diseases, dysfunctional uterine bleeding, anemia, pediatric aplastic anemia, endometriosis, polycystic ovary syndrome, and thyroid diseases. In some embodiments, the diseases or disorders include, but are not limited to, myeloma, including multiple myeloma, plasma cell myeloma, smoldering myeloma, smoldering multiple myeloma; myelofibrosis; bone marrow disease; myelodysplastic syndrome (MDS); previously treated myelodysplastic syndrome; transplantation-related cancer; neutropenia; leukemia, including chronic myelogenous leukemia, B-cell chronic lymphocytic leukemia, leukemia-associated anemia, acute myeloid leukemia (AML), chronic myeloid leukemia (CML), monocytic leukemia, myelomonocytic leukemia, acute lymphoblastic leukemia (ALL), acute T-lymphocytic leukemia, T-lymphocytic leukemia, chronic lymphocytic leukemia; lymphoma, including diffuse large B-cell lymphoma, non-Hodgkin's lymphoma, anaplastic lymphoma, anaplastic large cell lymphoma, immunoblastic T-Cell lymphoma, CD₂₀ positive lymphoma, mantle cell lymphoma, follicular lymphoma (FL), Burkitt's lymphoma, marginal zone lymphoma (MZL), primary lymphoma, B-cell lymphoma, recurrent B-cell non-Hodgkin's lymphoma, recurrent diffuse large B-cell lymphoma, recurrent mediastinal (thymic) large B-cell lymphoma, primary mediastinal (thymic) large B-cell lymphoma, recurrent transformed non-Hodgkin's lymphoma, refractory B-cell non-Hodgkin's lymphoma, refractory diffuse large B-cell lymphoma, refractory primary mediastinal (thymic) large B-cell lymphoma, refractory transformed non-Hodgkin's lymphoma; thyroid cancer; melanoma; lung cancer, including lung adenocarcinoma, lung squamous cell carcinoma, non-small cell lung cancer and small cell lung cancer; inflammatory myofibroblastoma; colorectal cancer; intestinal cancer; brain glioma; astroblastoma; glioma; peripheral neuroepithelioma; ovarian cancer; bronchial cancer; prostate cancer; breast cancer, including triple negative breast cancer, sporadic breast cancer, ductal carcinoma of the breast, and patients with Cowden syndrome; pancreatic cancer; central nervous system tumor; neuroblastoma; extramedullary plasmacytoma; plasmacytoma; gastric cancer; gastrointestinal stromal tumors; esophageal cancer; colorectal adenocarcinoma; esophageal squamous cell carcinoma; liver cancer; renal cell carcinoma; bladder cancer; endometrial cancer; head and neck cancer; brain cancer; oral cancer; sarcoma, including rhabdomyosarcoma, various lipogenic tumors, Ewing's sarcoma/primitive neuroectodermal tumors (Ewing/PNETs), and leiomyosarcoma; urothelial carcinoma; basal cell carcinoma; oral squamous cell carcinoma; cholangiocarcinoma; bone cancer; cervical cancer; skin cancer; Unverricht's syndrome; Richter syndrome (RS); sepsis syndrome; autoimmune diseases, including rheumatoid arthritis, autoimmune encephalomyelitis, ankylosing spondylitis, psoriasis, systemic lupus erythematosus, multiple sclerosis, recurrent oral ulcers, Kawasaki disease, polymyositis/dermatomyositis, Sjogren's syndrome, and atopic dermatitis; keratoconjunctivitis; autoinflammatory diseases, including gout, etc; inflammatory diseases, including Crohn's disease and ulcerative colitis, pneumonia, osteoarthritis, synovitis, systemic inflammatory response syndrome, airway inflammation, bronchitis; cerebral malaria; infectious diseases, including viral pneumonia, Acquired immunodeficiency syndrome (AIDS), COVID-19 novel coronavirus infection, gram-negative bacteria infection, gram-positive bacteria infection, tuberculosis, etc; septic shock; bacterial meningitis; chronic obstructive pulmonary disease; asthma; hemorrhagic shock; organ (including kidney, heart, lung) or tissue transplantation rejection; diabetes; sarcoidosis; adult respiratory distress syndrome; multiple organ dysfunction caused by cachexia and septic shock; acute liver failure; dysfunctional uterine bleeding; anemia; pediatric aplastic anemia; endometriosis; polycystic ovary syndrome; thyroid disease; cardiovascular diseases (e.g., coronary heart disease, congestive heart failure, myocardial infarction, atherosclerosis); skin diseases (e.g., acne); Kennedy disease; seborrheic alopecia; and hirsutism.

The present disclosure provides a method for treating and/or preventing a disease or disorder in a subject, comprising administering to the subject a therapeutically effective amount of the compound of Formula (I) or a pharmaceutically acceptable salt thereof, or the pharmaceutical composition of the present disclosure comprising as an active ingredient the compounds of Formula (I) of the present disclosure or a pharmaceutically acceptable salt thereof, wherein the diseases or disorders include, but are not limited to, tumors, autoinflammatory diseases, inflammatory diseases, autoimmune diseases, neurological disorders, respiratory diseases, hemorrhagic shock, transplant rejection, multiple organ dysfunction syndrome (MODS), sarcoidosis, adult respiratory distress syndrome, cardiovascular diseases, Richter syndrome (RS), acute liver failure, diabetes, Kennedy disease, seborrheic alopecia, hirsutism, skin diseases, dysfunctional uterine bleeding, anemia, pediatric aplastic anemia, endometriosis, polycystic ovary syndrome, and/or thyroid diseases. In some embodiments, the diseases or disorders include, but are not limited to, myeloma, including multiple myeloma, plasma cell myeloma, smoldering myeloma, smoldering multiple myeloma; myelofibrosis; bone marrow disease; myelodysplastic syndrome (MDS); previously treated myelodysplastic syndrome; transplantation-related cancer; neutropenia; leukemia, including chronic myelogenous leukemia, B-cell chronic lymphocytic leukemia, leukemia-associated anemia, acute myeloid leukemia (AML), chronic myeloid leukemia (CML), monocytic leukemia, myelomonocytic leukemia, acute lymphoblastic leukemia (ALL), acute T-lymphocytic leukemia, T-lymphocytic leukemia, chronic lymphocytic leukemia; lymphoma, including diffuse large B-cell lymphoma, non-Hodgkin's lymphoma, anaplastic lymphoma, anaplastic large cell lymphoma, immunoblastic T-Cell lymphoma, CD₂₀ positive lymphoma, mantle cell lymphoma, follicular lymphoma (FL), Burkitt's lymphoma, marginal zone lymphoma (MZL), primary lymphoma, B-cell lymphoma, recurrent B-cell non-Hodgkin's lymphoma, recurrent diffuse large B-cell lymphoma, recurrent mediastinal (thymic) large B-cell lymphoma, primary mediastinal (thymic) large B-cell lymphoma, recurrent transformed non-Hodgkin's lymphoma, refractory B-cell non-Hodgkin's lymphoma, refractory diffuse large B-cell lymphoma, refractory primary mediastinal (thymic) large B-cell lymphoma, refractory transformed non-Hodgkin's lymphoma; thyroid cancer; melanoma; lung cancer, including lung adenocarcinoma, lung squamous cell carcinoma, non-small cell lung cancer and small cell lung cancer; inflammatory myofibroblastoma; colorectal cancer; intestinal cancer; brain glioma; astroblastoma; glioma; peripheral neuroepithelioma; ovarian cancer; bronchial cancer; prostate cancer; breast cancer, including triple negative breast cancer, sporadic breast cancer, ductal carcinoma of the breast, and patients with Cowden syndrome; pancreatic cancer; central nervous system tumor; neuroblastoma; extramedullary plasmacytoma; plasmacytoma; gastric cancer; gastrointestinal stromal tumors; esophageal cancer; colorectal adenocarcinoma; esophageal squamous cell carcinoma; liver cancer; renal cell carcinoma; bladder cancer; endometrial cancer; head and neck cancer; brain cancer; oral cancer; sarcoma, including rhabdomyosarcoma, various lipogenic tumors, Ewing's sarcoma/primitive neuroectodermal tumors (Ewing/PNETs), and leiomyosarcoma; urothelial carcinoma; basal cell carcinoma; oral squamous cell carcinoma; cholangiocarcinoma; bone cancer; cervical cancer; skin cancer; Unverricht's syndrome; Richter syndrome (RS); sepsis syndrome; autoimmune diseases, including rheumatoid arthritis, autoimmune encephalomyelitis, ankylosing spondylitis, psoriasis, systemic lupus erythematosus, multiple sclerosis, recurrent oral ulcers, Kawasaki disease, polymyositis/dermatomyositis, Sjogren's syndrome, and atopic dermatitis; keratoconjunctivitis; autoinflammatory diseases, including gout, etc; inflammatory diseases, including Crohn's disease and ulcerative colitis, pneumonia, osteoarthritis, synovitis, systemic inflammatory response syndrome, airway inflammation, bronchitis; cerebral malaria; infectious diseases, including viral pneumonia, Acquired immunodeficiency syndrome (AIDS), COVID-19 novel coronavirus infection, gram-negative bacteria infection, gram-positive bacteria infection, tuberculosis, etc; septic shock; bacterial meningitis; chronic obstructive pulmonary disease; asthma; hemorrhagic shock; organ (including kidney, heart, lung) or tissue transplantation rejection; diabetes; sarcoidosis; adult respiratory distress syndrome; multiple organ dysfunction caused by cachexia and septic shock; acute liver failure; dysfunctional uterine bleeding; anemia; pediatric aplastic anemia; endometriosis; polycystic ovary syndrome; thyroid disease; cardiovascular diseases (e.g., coronary heart disease, congestive heart failure, myocardial infarction, atherosclerosis); skin diseases (e.g., acne); Kennedy disease; seborrheic alopecia; and hirsutism.

In the method for preventing and/or treating a disease or disorder (including a tumor or cancer) in a subject, the compound of Formula (I) of the present disclosure or the pharmaceutical composition comprising as an active ingredient the compound of Formula (I) of the present disclosure is administered to the subject through at least one mode of administration selected from the group consisting of nasal administration, inhalation administration, topical administration, oral administration, oral mucosal administration, rectal administration, pleural cavity administration, peritoneal administration, intramuscular administration, subcutaneous, transdermal, epidural, intrathecal, and intravenous administration.

The compound of Formula (II) of the present disclosure, or a pharmaceutically acceptable salt, solvate, isotopically enriched analog, polymorph, prodrug, stereoisomer (including enantiomer), or mixture of stereoisomers thereof, is a novel CRBN E3 ubiquitin ligase ligand, which can act as a molecular glue binding to the CRBN E3 ligase.

The compound of Formula (II) of the present disclosure, or a pharmaceutically acceptable salt, solvate, isotopically enriched analog, polymorph, prodrug, stereoisomer (including enantiomer), or mixture of stereoisomers thereof, can be used in the manufacture of a Cereblon protein (CRBN) binding agent.

The compound of Formula (II) of the present disclosure, or a pharmaceutically acceptable salt, solvate, isotopically enriched analog, polymorph, prodrug, stereoisomer (including enantiomer), or mixture of stereoisomers thereof, can be used for the preparation of the compound of Formula (I) of the present disclosure. The compound of Formula (I) of the present disclosure can be used for treating and/or preventing a disease or disorder selected from the group consisting of: tumors, autoinflammatory diseases, inflammatory diseases, autoimmune diseases, neurological disorders, respiratory diseases, hemorrhagic shock, transplant rejection, multiple organ dysfunction syndrome (MODS), sarcoidosis, adult respiratory distress syndrome, cardiovascular diseases, Richter syndrome (RS), acute liver failure, diabetes, Kennedy disease, seborrheic alopecia, hirsutism, skin diseases, dysfunctional uterine bleeding, anemia, pediatric aplastic anemia, endometriosis, polycystic ovary syndrome, and/or thyroid diseases.

The compound of Formula (II) of the present disclosure or a pharmaceutically acceptable salt, solvate, isotopically enriched analog, polymorph, prodrug, stereoisomer (including enantiomer), or mixture of stereoisomers thereof can be used as a medicament. Especially the compound of Formula (II) of the present disclosure or a pharmaceutically acceptable salt, solvate, isotopically enriched analog, polymorph, prodrug, stereoisomer (including enantiomer), or mixture of stereoisomers thereof can be used for the manufacture of a medicament for the prevention and/or treatment of a disease or disorder associated with a cereblon protein. The disease or disorder associated with a cereblon protein comprises: tumors, infectious disease, inflammatory diseases, autoimmune diseases, anemia, hemorrhagic shock, transplant rejection, multiple organ dysfunction syndrome (MODS), sarcoidosis, adult respiratory distress syndrome, cardiovascular diseases, Richter syndrome (RS), acute liver failure, or diabetes. In some embodiments, the disease or disorder associated with a cereblon protein include, but are not limited to, myeloma, including multiple myeloma, plasma cell myeloma, smoldering myeloma, smoldering multiple myeloma; myelofibrosis; bone marrow disease; myelodysplastic syndrome (MDS); previously treated myelodysplastic syndrome; transplantation-related cancer; neutropenia; leukemia, including chronic myelogenous leukemia, B-cell chronic lymphocytic leukemia, leukemia-associated anemia, acute myeloid leukemia (AML), chronic myeloid leukemia (CML), monocytic leukemia, myelomonocytic leukemia, acute lymphoblastic leukemia (ALL), acute T-lymphocytic leukemia, T-lymphocytic leukemia, chronic lymphocytic leukemia; lymphoma, including diffuse large B-cell lymphoma, non-Hodgkin's lymphoma, Hodgkin's lymphoma, anaplastic lymphoma, anaplastic large cell lymphoma, immunoblastic T-Cell lymphoma, CD20 positive lymphoma, mantle cell lymphoma, follicular lymphoma (FL), Burkitt's lymphoma, marginal zone lymphoma (MZL), primary lymphoma, B-cell lymphoma, recurrent B-cell non-Hodgkin's lymphoma, recurrent diffuse large B-cell lymphoma, recurrent mediastinal (thymic) large B-cell lymphoma, primary mediastinal (thymic) large B-cell lymphoma, recurrent transformed non-Hodgkin's lymphoma, refractory B-cell non-Hodgkin's lymphoma, refractory diffuse large B-cell lymphoma, refractory primary mediastinal (thymic) large B-cell lymphoma, refractory transformed non-Hodgkin's lymphoma; thyroid cancer; melanoma; lung cancer, including lung adenocarcinoma, lung squamous cell carcinoma; inflammatory myofibroblastoma; colorectal cancer; intestinal cancer; brain glioma; astroblastoma; ovarian cancer; bronchial cancer; prostate cancer; breast cancer, including triple negative breast cancer, sporadic breast cancer, ductal carcinoma of the breast, and patients with Cowden syndrome; pancreatic cancer; central nervous system tumor; neuroblastoma; glioma; peripheral neuroepithelioma; extramedullary plasmacytoma; plasmacytoma; gastric cancer; gastrointestinal stromal tumors; esophageal cancer; colorectal adenocarcinoma; esophageal squamous cell carcinoma; liver cancer; renal cell carcinoma; bladder cancer; endometrial cancer; metrocarcinoma; head and neck cancer; brain cancer; oral cancer; sarcoma, including rhabdomyosarcoma, various lipogenic tumors, Ewing's sarcoma/primitive neuroectodermal tumors (Ewing/PNETs), and leiomyosarcoma; urothelial carcinoma; basal cell carcinoma; oral squamous cell carcinoma; cholangiocarcinoma; bone cancer; cervical cancer; skin cancer; Richter syndrome (RS); sepsis syndrome; autoimmune diseases, including rheumatoid arthritis, autoimmune encephalomyelitis, ankylosing spondylitis, psoriasis, systemic lupus erythematosus, multiple sclerosis, recurrent oral ulcers, Kawasaki disease, polymyositis/dermatomyositis, Sjogren's syndrome, and atopic dermatitis; keratoconjunctivitis; inflammatory diseases, including Crohn's disease and ulcerative colitis, pneumonia, osteoarthritis, synovitis, systemic inflammatory response syndrome, airway inflammation, bronchitis; cerebral malaria; infectious diseases, including viral pneumonia, Acquired immunodeficiency syndrome (AIDS), COVID-19 novel coronavirus infection, gram-negative bacteria infection, gram-positive bacteria infection, tuberculosis, etc; septic shock; tuberculosis; bacterial meningitis; chronic obstructive pulmonary disease; asthma; hemorrhagic shock; organ (including kidney, heart, lung) or tissue transplantation rejection; diabetes; sarcoidosis; adult respiratory distress syndrome; anemia; pediatric aplastic anemia; cardiovascular diseases (e.g., coronary heart disease, congestive heart failure, myocardial infarction, atherosclerosis); multiple organ dysfunction caused by cachexia and septic shock; and acute liver failure.

The present disclosure provides a method for preventing and/or treating a disease or disorder associated with a cereblon protein in a subject, comprising administering to the subject a therapeutically effective amount of the compounds of Formula (II) of the present disclosure or a pharmaceutically acceptable salt thereof, or the pharmaceutical composition of the present disclosure comprising as an active ingredient the compounds of Formula (II) of the present disclosure or a pharmaceutically acceptable salt thereof. In some embodiments, the disease or disorder associated with a cereblon protein comprises: tumors, infectious disease, inflammatory diseases, autoimmune diseases, anemia, hemorrhagic shock, transplant rejection, multiple organ dysfunction syndrome (MODS), sarcoidosis, adult respiratory distress syndrome, cardiovascular diseases, Richter syndrome (RS), acute liver failure, or diabetes. In some embodiments, the disease or disorder associated with a cereblon protein include, but are not limited to, myeloma, including multiple myeloma, plasma cell myeloma, smoldering myeloma, smoldering multiple myeloma; myelofibrosis; bone marrow disease; myelodysplastic syndrome (MDS); previously treated myelodysplastic syndrome; transplantation-related cancer; neutropenia; leukemia, including chronic myelogenous leukemia, B-cell chronic lymphocytic leukemia, leukemia-associated anemia, acute myeloid leukemia (AML), chronic myeloid leukemia (CML), monocytic leukemia, myelomonocytic leukemia, acute lymphoblastic leukemia (ALL), acute T-lymphocytic leukemia, T-lymphocytic leukemia, chronic lymphocytic leukemia; lymphoma, including diffuse large B-cell lymphoma, non-Hodgkin's lymphoma, Hodgkin's lymphoma, anaplastic lymphoma, anaplastic large cell lymphoma, immunoblastic T-Cell lymphoma, CD20 positive lymphoma, mantle cell lymphoma, follicular lymphoma (FL), Burkitt's lymphoma, marginal zone lymphoma (MZL), primary lymphoma, B-cell lymphoma, recurrent B-cell non-Hodgkin's lymphoma, recurrent diffuse large B-cell lymphoma, recurrent mediastinal (thymic) large B-cell lymphoma, primary mediastinal (thymic) large B-cell lymphoma, recurrent transformed non-Hodgkin's lymphoma, refractory B-cell non-Hodgkin's lymphoma, refractory diffuse large B-cell lymphoma, refractory primary mediastinal (thymic) large B-cell lymphoma, refractory transformed non-Hodgkin's lymphoma; thyroid cancer; melanoma; lung cancer, including lung adenocarcinoma, lung squamous cell carcinoma; inflammatory myofibroblastoma; colorectal cancer; intestinal cancer; brain glioma; astroblastoma; ovarian cancer; bronchial cancer; prostate cancer; breast cancer, including triple negative breast cancer, sporadic breast cancer, ductal carcinoma of the breast, and patients with Cowden syndrome; pancreatic cancer; central nervous system tumor; neuroblastoma; glioma; peripheral neuroepithelioma; extramedullary plasmacytoma; plasmacytoma; gastric cancer; gastrointestinal stromal tumors; esophageal cancer; colorectal adenocarcinoma; esophageal squamous cell carcinoma; liver cancer; renal cell carcinoma; bladder cancer; endometrial cancer; metrocarcinoma; head and neck cancer; brain cancer; oral cancer; sarcoma, including rhabdomyosarcoma, various lipogenic tumors, Ewing's sarcoma/primitive neuroectodermal tumors (Ewing/PNETs), and leiomyosarcoma; urothelial carcinoma; basal cell carcinoma; oral squamous cell carcinoma; cholangiocarcinoma; bone cancer; cervical cancer; skin cancer; Richter syndrome (RS); sepsis syndrome; autoimmune diseases, including rheumatoid arthritis, autoimmune encephalomyelitis, ankylosing spondylitis, psoriasis, systemic lupus erythematosus, multiple sclerosis, recurrent oral ulcers, Kawasaki disease, polymyositis/dermatomyositis, Sjogren's syndrome, and atopic dermatitis; keratoconjunctivitis; inflammatory diseases, including Crohn's disease and ulcerative colitis, pneumonia, osteoarthritis, synovitis, systemic inflammatory response syndrome, airway inflammation, bronchitis; cerebral malaria; infectious diseases, including viral pneumonia, Acquired immunodeficiency syndrome (AIDS), COVID-19 novel coronavirus infection, gram-negative bacteria infection, gram-positive bacteria infection, tuberculosis, etc; septic shock; tuberculosis; bacterial meningitis; chronic obstructive pulmonary disease; asthma; hemorrhagic shock; organ (including kidney, heart, lung) or tissue transplantation rejection; diabetes; sarcoidosis; adult respiratory distress syndrome; anemia; pediatric aplastic anemia; cardiovascular diseases (e.g., coronary heart disease, congestive heart failure, myocardial infarction, atherosclerosis); multiple organ dysfunction caused by cachexia and septic shock; and acute liver failure.

In the method for preventing and/or treating diseases or disorders associated with cereblon protein, the compound of Formula (II) of the present disclosure or the pharmaceutical composition comprising as an active ingredient the compound of Formula (II) of the present disclosure is administered to the subject through at least one mode of administration selected from the group consisting of nasal administration, inhalation administration, topical administration, oral administration, oral mucosal administration, rectal administration, pleural cavity administration, peritoneal administration, intramuscular administration, subcutaneous, transdermal, epidural, intrathecal, and intravenous administration.

The term “treatment” or “treating” refers to administering to a subject the compound of the present disclosure (the compounds of Formula (I) or Formula (II)), or a pharmaceutically acceptable salt thereof, or the pharmaceutical composition comprising, as an active ingredient, the compound of the present disclosure (the compounds of Formula (I) or Formula (II)) or a pharmaceutically acceptable salt thereof, to mitigate (alleviate) undesirable diseases or conditions, such as the development of a cancer or tumor. The beneficial or desired clinical results of the present disclosure include, but are not limited to: alleviating symptoms, reducing the severity of the disease, stabilizing the state of the disease, slowing down or delaying the progression of the disease, improving or alleviating the condition, and alleviating the disease.

A “therapeutic effective amount” of the compound of the present disclosure depends on a variety of factors, including the activity of the specific compound used, the metabolic stability of the compound and the duration of its action, the age, sex and weight of the patient, the patient's current medical condition, the route and duration of administration, the excretion rate, the combined administration of additional drugs, and the progression of the diseases or conditions of the patient being treated. Those skilled in the art will be able to determine appropriate dosages based on these and other factors.

It is to be understood that the choice of using one or more active compounds and/or compositions and their dosage depends on the basic situations of the individual (which should generally render the individual situation to achieve the best effect). Dosing and dosing regimens should be within the ability of those skilled in the art, and the appropriate dosage depends on many factors including the knowledge and ability of the physicians, veterinarians or researchers (see e.g., Jun Li (chief editor), “Clinical Pharmacology”, 4th edition, People's Public Health Press, 2008).

As used herein, the term “patient” or “subject” to be treated refers to animal, for example mammal, including but not limited to primate (such as human being), cow, sheep, goat, horse, dog, cat, rabbit, guinea pig, rat, mice, etc.

### VI. DEFINITIONS

Unless otherwise specified, the following words, phrases and symbols used herein generally have the meanings as described below.

n general, the nomenclature used herein (including the IUPAC nomenclature) and the laboratory procedures described below (including those used in cell culture, organic chemistry, analytical chemistry, and pharmacology, etc.) are those well-known and commonly used in the art. Unless otherwise defined, all scientific and technical terms used herein in connection with the present disclosure described herein have the same meaning as commonly understood by one skill in the art. In addition, the use of the word “a” or “an” when used in conjunction with the term “comprising” or a noun in the claims and/or the specification may mean “one”, but it is also consistent with the meaning of “one or more”, “at least one”, and “one or more than one”. Similarly, the terms “another” or “other” can mean at least a second or more.

It should be understood that whenever the term “comprise” or “include” is used herein to describe various aspects, other similar aspects described by “consisting of” and/or “consisting essentially of” are also provided.

As used herein, the term “about” used alone or in combination refers to approximately, roughly, nearly, or around. When the term “about” is used in conjunction with a numerical range, it modifies that range by extending the boundaries above and below the stated numerical value. In general, the term “about” can modify a numerical value above and below the stated value by an upward or downward (increasing or decreasing) variation, e.g., 10%, 5%, 2%, or 1%.

As used herein, the wording “ . . . represents a bond” used alone or in combination means that the referenced group is a bond linker (that is, the referenced group is absent). For example, the wording “R_{w2} represents a bond” means that R_{w2} is a bond linker. In other words, when R_{w2} represents a bond, the R_{c} of PBM in the structure of Formula (I) is directly connected to the ring A⁵ in LIN. For example, the wording “R^{6a} in Formula (PBM-6) represents a bond” means that R^{6a} is a bond linker. In other words, when R^{6a} represents a bond, the nitrogen atom adjacent to R^{6a} in the structure of Formula (PBM-6) is directly linked to the carbon atom.

In this document, the term “optionally substituted” used alone or in combination means that the referenced group may be unsubstituted or substituted with one or more substituents as defined here. Herein, the wording “optionally substituted with . . . ” and “unsubstituted or substituted” can be used interchangeably. The term “substituted” generally represents the replacement of one or more hydrogen atoms in the referenced structure by identical or different specific substituents. The number of substituents is not theoretically limited in any way, or is automatically limited by the size of the building units (i.e., the total number of replaceable hydrogen atoms in the building units), or as clearly defined herein.

As used herein, the term “replaced” in the expression “any one or more methylene groups in the main carbon chain skeleton of a linear or branched C₁₋₂₀ alkylene are optionally be replaced by one or more groups selected from: Rₐ, R_{b}, and any combination thereof” used alone or in combination, has a definition known in the art, namely it may refer to one or more groups Rₐ, R_{b}, or any combination of groups Rₐ and R_{b} optionally replacing any one or more methylene groups in the main carbon chain skeleton of a C₁₋₂₀ alkylene. Herein, examples of the above-mentioned expression may include, but are not limited to, that any one or more (e.g., 1-20 or 1-15, 1-10, 1-9, 1-8, 1-7, 1-6, 1-5, 1-4, 1-3, or 1-2, or 1)methylene groups in the main carbon chain skeleton of a C₁₋₂₀ alkylene optionally are replaced by one or more (e.g., 1-20 or 1-15, 1-10, 1-9, 1-8, 1-7, 1-6, 1-5, 1-4, 1-3, or 1-2, or 1) groups Rₐ as defined herein, and/or one or more (e.g., 1-20 or 1-15, 1-10, 1-9, 1-8, 1-7, 1-6, 1-5, 1-4, 1-3, or 1-2, or 1) groups R_{b}, and/or one or more (e.g., 1-20 or 1-15, 1-10, 1-9, 1-8, 1-7, 1-6, 1-5, 1-4, 1-3, or 1-2, or 1) of any combination of groups Rₐ and R_{b}, and the resulting main chain skeleton group conforms to the covalent bond theory. When two or more methylene groups in the main carbon chain skeleton of the linear or branched C₁₋₂₀ alkylene are replaced by two or more groups Rₐ, the respective groups Rₐ are not directly connected to each other. When any one or more methylene groups in the main carbon chain skeleton of the linear or branched C₁₋₂₀ alkylene are replaced by one or more groups Rₐ, and/or one or more groups R_{b}, and/or one or more of any combination of groups Rₐ and R_{b}, the resulting main chain skeleton group may contain one or more (e.g., 1-20 or 1-15, 1-10, 1-9, 1-8, 1-7, 1-6, 1-5, 1-4, 1-3, or 1-2, or 1) of the following fragments, such as but not limited to: “—(CH₂)₀₋₅—Rₐ—(CH₂)₀₋₅-” fragment, “—Rₐ—(CH₂)₀₋₅—Rₐ-” fragment, “—(CH₂)₀₋₅-(R_{b})₀₋₅—(CH₂)_{0- 5}-” fragment, “—(R_{b})₀₋₅—(CH₂)₀₋₅-(R_{b})₀₋₅-” fragment, “—(CH₂)₀₋₅—Rₐ-(R_{b})₀₋₅—(CH₂)₀₋₅-” fragment, “—Rₐ—(CH₂)₀₋₅-(R_{b})₀₋₅—(CH₂)₀₋₅-” fragment, “—(CH₂)₀₋₅—Rₐ—(CH₂)₀₋₅-(R_{b})₀₋₅-” fragment, etc., wherein each Rₐ is the same or different, each R^{p} is the same or different, and as defined herein.

Herein, a bond interrupted by a wavy line shows the point of attachment of the depicted group to the rest of the molecule. For example, the group of Formula (ULM) depicted below

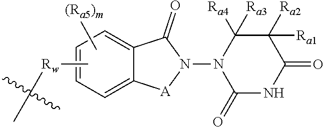

shows the point of attachment of R_{w} in said group to LIN of the compound of Formula (I). Herein, when a depicted group has two bonds interrupted by a wavy line, for example, when R_{w} represents

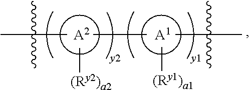

unless otherwise specified, it indicates that either end (e.g., ring A²) of said group may be connected to LIN, while the other end is connected to the isoindoline ring of Formula (ULM). Herein, when R_{c} represents the following group:

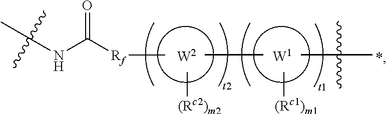

the symbol * indicates the point of attachment to LIN.

As used herein, the expression “one or more hydrogens of the C_{x-y} alkylene are replaced by . . . ”, used alone or in combination, means that any one or more hydrogens of the linear or branched C_{x-y} alkylene is replaced by a substituent(s) as defined herein. Herein, the term “one or more” may refer to part or all hydrogen atoms of each referenced alkylene group, including but not limited to 1-40 hydrogens, e.g., 1-30, such as 1-25, 1-20, 1-15, 1-10, 1-5, 1-4, 1-3, 1-2 or 1 hydrogen atoms. The number of hydrogens to be replaced is in principle not limited in any way, or is automatically limited by the size of the building unit.

As used herein, the term “deuterated” used alone or in combination, indicates that one or more hydrogen atoms in the referenced group are replaced by deuterium atoms.

As used herein, the term “oxo” or “oxo group” refers to ═O.

As used herein, the term “halogen atom” or “halogen”, used alone or in combination, refers to fluorine, chlorine, bromine, or iodine.

As used herein, the term “alkyl”, used alone or in combination, refers to a linear or branched alkyl group. The term “Cₓ-C_{y} alkyl” or “C_{x-y} alkyl” (x and y each being an integer) refers to a linear or branched alkyl group containing from x to y carbon atoms. The term “C₁₋₁₀ alkyl” used alone or in combination in the present disclosure refers to a linear or branched alkyl group containing from 1 to 10 carbon atoms. Examples of the C₁₋₁₀ alkyl of the present disclosure may include a C₁₋₉ alkyl, C₁₋₈ alkyl, C₂₋₈ alkyl, C₁₋₇ alkyl, C₁₋₆ alkyl, C₁₋₅ alkyl, C₁₋₄ alkyl, and C₁₋₃ alkyl. Representative examples include methyl, ethyl, n-propyl, isopropyl, n-butyl, isobutyl, sec-butyl, tert-butyl, pentyl, isopentyl, neopentyl, tert-pentyl, hexyl, heptyl, octyl, nonyl, and decyl. The term “C₁₋₃ alkyl” or “C₁-C₃ alkyl” in the present disclosure refers to an alkyl group containing from 1 to 3 carbon atoms, and representative examples thereof include methyl, ethyl, n-propyl, and isopropyl. In the present disclosure, the “alkyl” is optionally substituted with one or more substituents optionally selected from the group consisting of halogen, hydroxyl, cyano, C₁₋₃ alkyl, C₁₋₃ alkoxy, trifluoromethyl, 4-to 15-membered heterocyclyl, or a combination thereof.

As used herein, the term “halogenated alkyl”, used alone or in combination, refers to a linear or branched alkyl group substituted with one or more halogens, wherein one or more hydrogen atom(s) of the alkyl group are replaced with one or more halogens. The term “halogenated Cₓ-C_{y} alkyl” or “halogenated C_{x-y} alkyl” (x and y are each an integer) refers to a linear or branched alkyl containing from x to y carbon atoms substituted with one or more halogens. The term “halogenated C₁₋₁₀ alkyl” used alone or in combination in the present disclosure refers to a linear or branched alkyl group containing from 1 to 10 carbon atoms substituted with one or more halogens. Examples of the halogenated C₁₋₁₀ alkyl group of the present disclosure include halogenated C₁₋₉ alkyl group, e.g., halogenated C₁₋₈ alkyl group, halogenated C₂₋₈ alkyl group, halogenated C₁₋₇ alkyl group, halogenated C₁₋₆ alkyl, halogenated C₁₋₅ alkyl, or halogenated C₁₋₄ alkyl. Representative examples include halomethyl, haloethyl, halo-n-propyl, haloisopropyl, halo-n-butyl, haloisobutyl, halo-sec-butyl, halo-tert-butyl, halopentyl, haloisoamyl, haloneopentyl, halo-tert-pentyl, halohexyl, haloheptyl, halooctyl, halononyl, and halodecyl. The term “halogenated C₁₋₃ alkyl” or “halogenated C₁-C₃ alkyl” of the present disclosure refers to an alkyl group containing from 1 to 3 carbon atoms substituted with one or more halogens, and its representative examples include halomethyl, haloethyl, halo-n-propyl and haloisopropyl.

As used herein, the term “deuterated alkyl”, used alone or in combination, refers to a linear or branched alkyl group substituted with one or more deuterium atoms, wherein one or more hydrogen atoms of the alkyl group are replaced with one or more deuterium atoms. The term “deuterated Cₓ-C_{y} alkyl” or “deuterated C_{x-y} alkyl” (x and y are each an integer) refers to a linear or branched alkyl containing from x to y carbon atoms substituted with one or more deuterium atoms. The term “deuterated C₁₋₁₀ alkyl” used alone or in combination in the present disclosure refers to a linear or branched alkyl group containing from 1 to 10 carbon atoms substituted with one or more deuterium atoms. Examples of the deuterated C₁₋₁₀ alkyl group of the present disclosure include deuterated C₁₋₉ alkyl group, e.g., deuterated C₁₋₈ alkyl group, deuterated C₂₋₈ alkyl group, deuterated C₁₋₇ alkyl group, deuterated C₁₋₆ alkyl, deuterated C₁₋₅ alkyl, or deuterated C₁₋₄ alkyl. Representative examples include perdeuterated methyl (CD₃), perdeuterated ethyl (CD₃CD₂), perdeuterated n-propyl, perdeuterated isopropyl, perdeuterated n-butyl, perdeuterated isobutyl, perdeuterated sec-butyl, perdeuterated tert-butyl, perdeuterated pentyl, perdeuterated isopentyl, perdeuterated neopentyl, perdeuterated tert-pentyl, perdeuterated hexyl, perdeuterated heptyl, perdeuterated octyl, perdeuterated nonyl, and perdeuterated decyl. The term “deuterated C₁-3 alkyl” or “deuterated C₁-C₃ alkyl” of the present disclosure refers to an alkyl group containing from 1 to 3 carbon atoms substituted with one or more deuterium atoms, and its representative examples include perdeuterated methyl (CD₃) and perdeuterated ethyl (CD₃CD₂).

As used herein, the term “alkylene” (which is used interchangeably with “alkylene chain”), used alone or in combination, refers to a linear or branched divalent saturated hydrocarbon group composed of carbon and hydrogen atoms. The term “Cₓ-C_{y} alkylene” or “C_{x-y} alkylene” (x and y each being an integer) refers to a linear or branched alkylene group containing from x to y carbon atoms. Examples of the C₁-C₁₅ alkylene in the present disclosure may include C₁-C₁₅ alkylene, C₁-C₁₄ alkylene, C₁-C₁₃ alkylene, C₁-C₁₂ alkylene, C₁-C₁₁ alkylene, C₁-C₁₀ alkylene, C₁-C₉ alkylene, C₁-C₈ alkylene, C₁-C₇ alkylene, C₁-C₆ alkylene, C₁-C₅ alkylene, C₁-C₄ alkylene, C₁-C₃ alkylene, or C₁-C₂ alkylene. Representative examples include, but are not limited to, methylene, ethylene, propylene, isopropylene, butylene, isobutylene, sec-butylene, tert-butylene, n-pentylene, isopentylene, neopentylidene, tert-pentylene, hexylene, heptylene, octylene, nonylene, decylene, undecylene, dodecylene, tridecylene, tetradecylene, and pentadecylene. In the present disclosure, the “alkylene” is optionally substituted with one or more substituents optionally selected from C₁₋₃ alkyl, C₃₋₆ cycloalkyl, hydroxyl, amino, mercapto, halogen, C₁₋₃ alkoxy, C₁₋₃ alkylamino, halogenated C₁₋₃ alkyl, amino-substituted C₁₋₃ alkylene, C₁₋₃ alkyl-NHC(O)—, C₁₋₃ alkyl-C(O)NH—, cyano, or any combination thereof.

As used herein, the term “alkoxy”, used alone or in combination, refers to a linear or branched alkoxy group having structural formula of alkyl-O—. Optionally, the alkyl portion of the alkoxy group may contain 1-10 carbon atoms. Representative examples of “alkoxy” include, but are not limited to, methoxy, ethoxy, propoxy, isopropoxy, n-butoxy, isobutoxy, tert-butoxy, pentyloxy, 2-pentyloxy, isopentyloxy, neopentyloxy, hexyloxy, 2-hexyloxy, 3-hexyloxy, 3-methylpentyloxy, etc. The term “C₁-C₃ alkoxy” or “C₁₋₃ alkoxy” refers to a linear or branched alkoxy group containing from 1 to 3 carbon atoms. Representative examples of C₁₋₃ alkoxy include, but are not limited to, methoxy, ethoxy, n-propoxy, and isopropoxy.

As used herein, the term “halogenated alkoxy”, used alone or in combination, refers to an alkoxy group substituted with one or more halogen atoms. Optionally, the alkyl portion of the alkoxy group may contain 1-10 (e.g., 1-6, 1-4, or 1-3) carbon atoms. Examples of “halogenated alkoxy” include, but are not limited to, halogenated C₁₋₆ alkoxy or halogenated C₁₋₄ alkoxy. Representative examples include, but are not limited to, F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O— or CH₂ClCH₂—O—.

As used herein, the term “alkylamino”, used alone or in combination, refers to a linear or branched alkylamino group having structural formula of alkyl-NH—. Optionally, the alkyl portion of the alkylamino group may contain 1-10 (e.g., 1-6, 1-4, or 1-3) carbon atoms. Representative examples of “alkylamino” include, but are not limited to, methyl-NH—, ethyl-NH—, n-propyl-NH—, isopropyl-NH—, n-butyl-NH—, isobutyl-NH—, tert-butyl-NH—, pentyl-NH—, and hexyl-NH—, etc. The term “C₁-C₃ alkyl-NH—” or “C₁₋₃ alkyl-NH—” refers to a linear or branched alkyl-NH— group containing from 1 to 3 carbon atoms. Representative examples of C₁₋₃ alkyl-NH— include, but are not limited to, methyl-NH—, ethyl-NH—, n-propyl-NH—, and isopropyl-NH—.

As used herein, the term “amino-substituted alkylene”, used alone or in combination, refers to a linear or branched alkylene group substituted with amino having structural formula of NH₂-alkylene. Optionally, the alkylene portion of the amino-substituted alkylene group may contain 1-10 (e.g., 1-6, 1-4, or 1-3) carbon atoms. The term “amino-substituted C₁₋₃ alkylene” or “NH₂-C₁₋₃ alkyl” refers to a linear or branched alkylene group containing from 1 to 3 carbon atoms which is substituted with amino. Representative examples of amino-substituted C₁₋₃ alkylene include, but are not limited to, NH₂—CH₂—, NH₂—CH₂CH₂—, and NH₂—CH₂CH₂CH₂—.

As used herein, the term “alkyl-NHC(O)—”, used alone or in combination, refers to a linear or branched alkyl-NHC(O)— group having structural formula of alkyl-NHC(O)—. Optionally, the alkyl portion of the alkyl-NHC(O)— group may contain 1-10 (e.g., 1-6, 1-4, or 1-3) carbon atoms. The term “C₁₋₃ alkyl-NHC(O)—” or “C₁-C₃ alkyl-NHC(O)—” refers to a linear or branched alkyl-NHC(O)— group containing from 1 to 3 carbon atoms. Representative examples of C₁₋₃ alkyl-NHC(O)-include, but are not limited to, CH₃—NHC(O)—, CH₃CH₂—NHC(O)—, and CH₃CH₂CH₂—NHC(O)—.

As used herein, the term “alkyl-C(O)NH—”, used alone or in combination, refers to a linear or branched alkyl-C(O)NH— group having structural formula of alkyl-C(O)NH—. Optionally, the alkyl portion of the alkyl-C(O)NH— group may contain 1-10 (e.g., 1-6, 1-4, or 1-3) carbon atoms. The term “C₁₋₃ alkyl-C(O)NH—” or “C₁-C₃ alkyl-C(O)NH—” refers to a linear or branched alkyl-C(O)NH— group containing from 1 to 3 carbon atoms. Representative examples of C₁₋₃ alkyl-C(O)NH-include, but are not limited to, CH₃—C(O)NH—, CH₃CH₂—C(O)NH—, and CH₃CH₂CH₂—C(O)NH—.

As used herein, the term “heteroaryl”, used alone or in combination, refers to a 5-to 20-membered (e.g., 5-to 15-membered, 5-to 12-membered, 5-to 11-membered, 5-to 10-membered, 5-to 9-membered, 5-to 8-membered, 5-to 7-membered, 5-to 6-membered, 6-to 15-membered, or 6-to 9-membered) monocyclic, bicyclic, or polycyclic cyclic hydrocarbon radical containing at least one aromatic ring having one or more (e.g., from 1 to 6, or from 1 to 5, or from 1 to 4, or from 1 to 3) heteroatoms independently selected from the group consisting of oxygen, nitrogen, and sulfur. Bicyclic or polycyclic heteroaryl groups include bicyclic, tricyclic or tetracyclic heteroaryl groups, which contain one aromatic ring having one or more heteroatoms independently selected from O, S and N, and the remaining rings which may be a saturated, partially unsaturated or aromatic ring and can be carbocyclic ring or contain one or more heteroatoms independently selected from O, S and N. Examples of monocyclic heteroaryl groups include, but are not limited to, furanyl, oxazolyl, isoxazolyl, oxadiazolyl, thienyl, thiazolyl, isothiazolyl, thiadiazolyl, pyrrolyl, imidazolyl, pyrazolyl, triazolyl, pyridyl, pyrimidinyl, pyridazinyl, pyrazinyl, tetrazolyl, and triazinyl. Examples of bicyclic heteroaryl groups include, but are not limited to, indolyl, isoindolyl, isoindolinyl, benzofuranyl, isobenzofuranyl, benzothienyl, indazolyl, benzimidazolyl, benzoxazolyl, benzisoxazolyl, benzothiazolyl, benzisothiazolyl, benzotriazolyl, benzo[2,1,3]oxadiazolyl, benzo[2,1,3]thiadiazolyl, benzo[1,2,3]thiadiazolyl, quinolinyl, isoquinolinyl, naphthyridinyl, cinnolinyl, quinazolinyl, quinoxalinyl, phthalazinyl, oxazolopyridyl, furopyridyl, pteridyl, purinyl, pyridopyridyl, pyrazolo[1,5-a]pyridyl, pyrazolo[1,5-a]pyrimidinyl, imidazo[1,2-a]pyridyl, 1H-pyrrolo[3,2-b]pyridyl, 1H-pyrrolo[2,3-b]pyridyl, pyrrolo[2,1-b]thiazolyl, imidazo[2,1-b]thiazolyl, chromanyl, and 6,7-dihydrothieno[3,2-d]pyrimidinyl. Examples of tricyclic or polycyclic heteroaryl groups include, but are not limited to, acridinyl, benzindolyl, carbazolyl, dibenzofuranyl, xanthyl, 5,6,7,8-tetrahydrobenzo[4,5]thieno[2,3-d]pyrimidinyl, and 6,7-dihydro-5H-cyclopenta[4,5]thieno[2,3-d]pyrimidinyl. The heteroaryl group may be unsubstituted or substituted. A substituted heteroaryl refers to heteroaryl substituted one or more times (e.g., 1-4, 1-3, or 1-2 times) by a substituent(s) optionally selected from the group consisting of C₁₋₃ alkyl, C₃₋₆ cycloalkyl, hydroxyl, amino, mercapto, halogen, C₁₋₃ alkoxy, C₁₋₃ alkylamino, halogenated C₁₋₃ alkyl, amino-substituted C₁₋₃ alkylene, C₁₋₃ alkyl-NHC(O)—, C₁₋₃ alkyl-C(O)NH—, cyano, or any combination thereof.

As used herein, the term “heteroarylene”, used alone or in combination, refers to a 5-to 20-membered (e.g., 5-to 15-membered, 5-to 12-membered, 5-to 11-membered, 5-to 10-membered, 5-to 9-membered, 5-to 8-membered, 5-to 7-membered, 5-to 6-membered, 6-to 15-membered, or 6-to 9-membered) bivalent monocyclic, bicyclic, or polycyclic cyclic hydrocarbon radical containing at least one aromatic ring having one or more (e.g., from 1 to 6, or from 1 to 5, or from 1 to 4, or from 1 to 3) heteroatoms independently selected from the group consisting of oxygen, nitrogen, and sulfur. Bicyclic or polycyclic heteroarylene groups include bicyclic, tricyclic or tetracyclic heteroarylene groups, which contain one aromatic ring having one or more heteroatoms independently selected from O, S and N, and the remaining ring(s) which may be a saturated, partially unsaturated or aromatic ring and can be carbocyclic ring or contain one or more heteroatoms independently selected from O, S and N. Examples of monocyclic heteroarylene groups include, but are not limited to, furanylene, oxazolylene, isoxazolylene, oxadiazolylene, thienylene, thiazolylene, isothiazolylene, thiadiazolylene, pyrrolylene, imidazolylene, pyrazolylene, triazolylene, pyridylene, pyrimidinylene, pyridazinylene, pyrazinylene, tetrazolylene, and triazinylene. Examples of bicyclic heteroarylene groups include, but are not limited to, indolylene, isoindolylene, isoindolinylene, benzofuranylene, isobenzofuranylene, benzothienylene, indazolylene, benzimidazolylene, benzoxazolylene, benzisoxazolylene, benzothiazolylene, benzisothiazolylene, benzotriazolylene, benzo[2,1,3]oxadiazolylene, benzo[2,1,3]thiadiazolylene, benzo[1,2,3]thiadiazolylene, quinolinylene, isoquinolinylene, naphthyridinylene, cinnolinylene, quinazolinylene, quinoxalinylene, phthalazinylene, oxazolopyridylene, furopyridylene, pteridylene, purinylene, pyridopyridylene, pyrazolo[1,5-alpyridylene, pyrazolo[1,5-a]pyrimidinylene, imidazo[1,2-a]pyridylene, 1H-pyrrolo[3,2-b]pyridylene, 1H-pyrrolo[2,3-b]pyridylene, pyrrolo[2,1-b]thiazolylene, imidazo[2,1-b]thiazolylene, chromanylene, and 6,7-dihydrothieno[3,2-d]pyrimidinylene. Examples of tricyclic or polycyclic heteroarylene groups include, but are not limited to, acridinylene, benzindolylene, carbazolylene, dibenzofuranylene, xanthylene, 5,6,7,8-tetrahydrobenzo[4,5]thieno[2,3-d]pyrimidinylene, and 6,7-dihydro-5H-cyclopenta[4,5]thieno[2,3-d]pyrimidinylene. The heteroarylene group may be unsubstituted or substituted. A substituted heteroarylene refers to heteroarylene substituted one or more times (e.g., 1-4, 1-3, or 1-2 times) by a substituent(s) optionally selected from the group consisting of C₁₋₃ alkyl, C₃₋₆ cycloalkyl, hydroxyl, amino, mercapto, halogen, C₁₋₃ alkoxy, C₁₋₃ alkylamino, halogenated C₁₋₃ alkyl, amino-substituted C₁₋₃ alkylene, C₁₋₃ alkyl-NHC(O)—, C₁₋₃ alkyl-C(O)NH—, cyano, or any combination thereof.

As used herein, the term “aryl”, used alone or in combination, refers to a monovalent aromatic hydrocarbon group containing from 5 to 20 (e.g., from 5 to 14, from 5 to 15, from 5 to 10, from 5 to 9, from 5 to 8, from 6 to 15, or from 6 to 10) carbon atoms and optionally one or more fused rings, such as phenyl group, naphthyl group, or fluorenyl group. As used herein, the “aryl” is optionally substituted. A substituted aryl group refers to an aryl group substituted one or more times (e.g., 1-4, 1-3, or 1-2 times) with a substituent(s). For example, aryl is mono-, di-, tri-, or poly-substituted with a substituent(s) optionally selected from e.g., C₁₋₃ alkyl, C₃₋₆ cycloalkyl, hydroxyl, amino, mercapto, halogen, C₁₋₃ alkoxy, C₁₋₃ alkylamino, halogenated C₁₋₃ alkyl, amino-substituted C₁₋₃ alkylene, C₁₋₃ alkyl-NHC(O)—, C₁₋₃ alkyl-C(O)NH—, cyano, or any combination thereof.

As used herein, the term “arylene”, used alone or in combination, refers to a divalent aromatic hydrocarbon group containing from 5 to 20 (e.g., from 5 to 14, from 5 to 15, from 5 to 10, from 5 to 9, from 5 to 8, from 6 to 15, or from 6 to 10) carbon atoms and optionally one or more fused rings, such as phenylene, naphthylene, or fluorenylene. As used herein, the “arylene” is optionally substituted. A substituted arylene refers to an arylene group substituted one or more times (e.g., 1-4, 1-3, or 1-2 times) with a substituent(s). For example, arylene is mono-, di-, tri-, or poly-substituted with a substituent(s) optionally selected from e.g., C₁₋₃ alkyl, C₃₋₆ cycloalkyl, hydroxyl, amino, mercapto, halogen, C₁₋₃ alkoxy, C₁₋₃ alkylamino, halogenated C₁₋₃ alkyl, amino-substituted C₁₋₃ alkylene, C₁₋₃ alkyl-NHC(O)—, C₁₋₃ alkyl-C(O)NH—, cyano, or any combination thereof.

As used herein, the term “cycloalkyl”, used alone or in combination, refers to a saturated or partially unsaturated (i.e., containing one or more double bonds, but not having a fully conjugated π-electron system) monocyclic or bicyclic or tricyclic or polycyclic cyclic hydrocarbon radical, which in some embodiments contains from 3 to 20 carbon atoms (i.e., C₃₋₂₀ cycloalkyl), or from 3 to 15 carbon atoms (i.e., C₃₋₁₅ cycloalkyl), or from 3 to 12 carbon atoms (i.e., C₃₋₁₂ cycloalkyl), or from 3 to 11 carbon atoms (i.e., C₃₋₁₁ cycloalkyl), or from 3 to 10 carbon atoms (i.e., C₃₋₁₀ cycloalkyl), or from 3 to 8 carbon atoms (i.e., C₃₋₈ cycloalkyl), or from 3 to 7 carbon atoms (i.e., C₃₋₇ cycloalkyl), or from 3 to 6 carbon atoms (i.e., C₃₋₆ cycloalkyl). The term “cycloalkyl” includes monocyclic, bicyclic, tricyclic, and polycyclic cyclic hydrocarbon radical having from 3 to 20 carbon atoms. Examples of the term “cycloalkyl” include, but are not limited to, monocyclic cycloalkyl, bridged cycloalkyl (e.g., C₅₋₁₅ bridged cycloalkyl or C₇₋₁₅ bridged cycloalkyl), fused cycloalkyl, and spiro-cycloalkyl (e.g., C₅₋₁₅ spiro-cycloalkyl). Representative examples of monocyclic cycloalkyl groups include, but are not limited to, cyclopropyl, cyclobutyl, cyclopentyl, cyclopentenyl, cyclohexyl, cyclohexenyl, cycloheptyl, and cyclooctyl. Examples of bridged cycloalkyl, fused cycloalkyl and spiro-cycloalkyl groups include, but are not limited to, decalinyl, octahydropentalenyl, octahydro-1H-indenyl, C₅₋₁₅ spiro-cycloalkyl, adamantanyl, noradamantanyl, bornyl, norbornyl (also named as bicyclo[2.2.1]heptyl by the IUPAC system). As used herein, the “cycloalkyl” is optionally mono- or poly-substituted, such as, but not limited to, 2,2-, 2,3-, 2,4-, 2,5-, or 2,6-disubstituted cyclohexyl. The substituents of the substituted “cycloalkyl” can be optionally one or more (e.g., 1-5, 1-4, 1-3, 1-2, or 1) substituents selected from C₁₋₃ alkyl, C₃₋₆ cycloalkyl, hydroxyl, amino, mercapto, halogen, C₁₋₃ alkoxy, C₁₋₃ alkylamino, halogenated C₁₋₃ alkyl, amino-substituted C₁₋₃ alkylene, C₁₋₃ alkyl-NHC(O)—, C₁₋₃ alkyl-C(O)NH—, cyano, or any combination thereof. Examples of “C₃₋₆ cycloalkyl” include, but are not limited to, cyclopropyl, cyclobutyl, cyclopentyl, cyclopentenyl, and cyclohexyl.

As used herein, the term “C_{x-y} spiro-cycloalkyl” (x and y each being an integer), used alone or in combination, refers to a spiro-cycloalkyl group containing from x to y carbon atoms. As used herein, the term “C₅₋₁₅ spiro-cycloalkyl”, used alone or in combination, refers to a spiro-cycloalkyl group containing from 5 to 15 carbon atoms (e.g., 7-15, 5-11, 5-10, or 7-9 carbon atoms). The term “C₅₋₁₅ spirocycloalkyl” includes “C₇₋₁₅ spirocycloalkyl”, and representative examples thereof include, but are not limited to, spiro[3.3]heptanyl, spiro[2.5]octanyl, spiro[3.5]nonanyl, spiro[4.4]nonanyl, spiro[4.5]decanyl, and spiro[5.5]undecanyl. The “C₅₋₁₅ spiro-cycloalkyl” is optionally further substituted with one or more substituents selected from the group consisting of C₁₋₃ alkyl, halogenated C₁₋₃ alkyl, C₁₋₃ alkoxy, deuterated C₁₋₃ alkoxy, C₁₋₃ alkylamino, amino, oxo, halogen, hydroxy, cyano, or any combination thereof.

As used herein, the term “C_{x-y} bridged cycloalkyl” (where x and y are integers) used alone or in combination, refers to a bridged cycloalkyl group containing x to y carbon atoms. In the present invention, the term “C₅₋₁₅ bridged cycloalkyl” used alone or in combination, refers to a bridged cycloalkyl group containing 5 to 15 carbon atoms. Representative examples of the term “C₅₋₁₅ bridged cycloalkyl” include, but are not limited to, adamantanyl, noradamantanyl, norbornanyl (systematically named bicyclo[2.2.1]heptanyl), and cubanyl. Said “bridged cycloalkyl” may be optionally substituted with 1 to 10 substituents selected from: C₁₋₃ alkyl, deuterated C₁₋₃ alkyl, C₁₋₃ alkoxy, halogen, halogenated C₁₋₃ alkyl, C₁₋₃ alkylamino, amino, hydroxy, cyano, oxo, or any combination thereof.

As used herein, the term “cycloalkylene”, used alone or in combination, refers to a saturated or partially unsaturated (i.e., containing one or more double bonds, but not having a fully conjugated π-electron system) monocyclic, bicyclic, tricyclic or polycyclic divalent cyclic hydrocarbon radical, which in some embodiments contains from 3 to 20 carbon atoms (i.e., C₃₋₂₀ cycloalkylene), or from 3 to 15 carbon atoms (i.e., C₃₋₁₅ cycloalkylene), or from 3 to 12 carbon atoms (i.e., C₃₋₁₂ cycloalkylene), or from 3 to 11 carbon atoms (i.e., C₃₋₁₁ cycloalkylene), or from 3 to 10 carbon atoms (i.e., C₃₋₁₀ cycloalkylene), or from 3 to 8 carbon atoms (i.e., C₃₋₈ cycloalkylene), or from 3 to 7 carbon atoms (i.e., C₃₋₇ cycloalkylene), or from 3 to 6 carbon atoms (i.e., C₃₋₆ cycloalkylene). The term “cycloalkylene” includes monocyclic, bicyclic, tricyclic and polycyclic divalent cyclic hydrocarbon radical having from 3 to 20 carbon atoms. Examples of the term “cycloalkylene” include, but are not limited to, monocyclic cycloalkylene, bridged cycloalkylene (e.g., C₅₋₁₅ bridged cycloalkylene or C₇₋₁₅ bridged cycloalkylene), fused cycloalkylene (e.g., C₅₋₁₅ fused cycloalkylene), and spiro-cycloalkylene (e.g., C₅₋₁₅ spiro-cycloalkylene). Representative examples of monocyclic cycloalkylene groups include, but are not limited to, cyclopropylene, cyclobutylene, cyclopentylene, cyclopentenylene, cyclohexylene, cyclohexenylene, cycloheptylene, and cyclooctylene. Examples of bridged cycloalkylene, fused cycloalkylene and spiro-cycloalkylene groups include, but are not limited to, decalinylene, octahydropentalenylene, octahydro-1H-indenylene, 2,3-dihydro-1H-indenylene, spiro-cycloalkylene (e.g., C₅₋₁₅ spiro-cycloalkylene), adamantanylene, noradamantanylene, and norbornylene (also named as bicyclo[2.2.1]heptylene by the IUPAC system). As used herein, the “cycloalkylene” is optionally mono- or poly-substituted, such as, but not limited to, 2,2-, 2,3-, 2,4-, 2,5-, or 2,6-disubstituted cyclohexylene. The substituents of the substituted “cycloalkylene” can be optionally one or more (e.g., 1-5, 1-4, 1-3, 1-2, or 1) substituents selected from the group consisting of C₁-C₃ alkyl, C₃₋₆ cycloalkyl, hydroxy, amino, mercapto, halogen, C₁-C₃ alkoxy, C₁-C₃ alkylamino, halogenated C₁-C₃ alkyl, amino-substituted C₁₋₃ alkylene, C₁₋₃ alkyl-NHC(O)—, C₁₋₃ alkyl-C(O)NH—, cyano or any combination thereof.

As used herein, the term “C_{x-y} spiro-cycloalkylene” (x and y each being an integer), used alone or in combination, refers to a spiro-cycloalkylene group containing from x to y carbon atoms. As used herein, the term “C₅₋₁₅ spiro-cycloalkylene”, used alone or in combination, refers to a spiro-cycloalkylene group containing from 5 to 15 carbon atoms (e.g., 7-15, 5-11, 5-10, or 7-9 carbon atoms). Representative examples of “C₅₋₁₅ spiro-cycloalkylene” include, but are not limited to, spiro[3.3]heptylene, spiro[2.5]octylene, spiro[3.5]nonylene, spiro[4.4]nonylene, spiro[4.5]decylene, or spiro[5.5]undecylene. The “C₅₋₁₅ spiro-cycloalkylene” is optionally further substituted with one or more substituents selected from the group consisting of C₁₋₃ alkyl, C₃₋₆ cycloalkyl, hydroxyl, amino, mercapto, halogen, C₁₋₃ alkoxy, C₁₋₃ alkylamino, halogenated C₁₋₃ alkyl, amino-substituted C₁-3 alkylene, C₁₋₃ alkyl-NHC(O)—, C₁₋₃ alkyl-C(O)NH—, cyano, or any combination thereof.

As used herein, the term “C_{x-y} bridged cycloalkylene” (where x and y are integers) used alone or in combination, refers to a bridged cycloalkylene group containing x to y carbon atoms. In the present invention, the term “C₅₋₁₅ bridged cycloalkylene” used alone or in combination, refers to a bridged cycloalkylene group containing 5 to 15 (e.g., 7-15, 5-11, 5-10, or 7-9) carbon atoms. Representative examples of the term “C₅₋₁₅ bridged cycloalkylene” include, but are not limited to, adamantanylene, noradamantanylene, norbornanylene, bicyclo[2.2.1]heptanylene, 2-oxobicyclo[2.2.1]heptanylene and bicyclo[2.2.1]heptenylene. Said “C₅₋₁₅ bridged cycloalkylene” may be optionally substituted with one or more substituents selected from: C₁₋₃ alkyl, C₃₋₆ cycloalkyl, hydroxyl, amino, mercapto, halogen, C₁₋₃ alkoxy, C₁₋₃ alkylamino, halogenated C₁₋₃ alkyl, amino-substituted C₁₋₃ alkylene, C₁₋₃ alkyl-NHC(O)—, C₁₋₃ alkyl-C(O)NH—, cyano, or any combination thereof.

As used herein, the term “heterocyclyl” or “heterocyclic group”, used alone or in combination, refers to a 4-to 20-membered (e.g., 4-to 15-membered, 4-to 14-membered, 4-to 13-membered, 4-to 12-membered, 4-to 11-membered, 4-to 10-membered, 4-to 9-membered, 4-to 8-membered, 4-to 7-membered, 4-to 6-membered, 4-to 5-membered, or 4-to 9-membered) saturated or partially unsaturated (i.e., containing one or more double bonds, but not having a fully conjugated π-electron system) monocyclic, bicyclic, tricyclic or polycyclic cyclic hydrocarbon group containing one or more (e.g., from 1 to 5, or from 1 to 4, from 1 to 3, from 1 to 2, or 1) heteroatoms independently selected from sulfur, oxygen, and nitrogen. Examples of the heterocyclyl groups include, but not limited to, monocyclic heterocyclyl (e.g., 4-to 20-membered monocyclic heterocyclyl), bridged heterocyclyl (e.g., 5-to 20-membered bridged heterocyclyl, and 7-to 15-membered bridged heterocyclyl), fused heterocyclyl (e.g., 5-to 20-membered fused heterocyclyl), and spiro-heterocyclyl groups (e.g., 5-to 20-membered spiro-heterocyclyl). Representative examples of the monocyclic heterocyclyl include, but are not limited to, azetidinyl, oxetanyl, pyrrolidinyl, imidazolidinyl, pyrazolidyl, tetrahydrofuranyl, tetrahydropyranyl, tetrahydrothienyl, tetrahydrothiopyranyl, oxazolidinyl, thiazolidinyl, piperidinyl, piperazinyl, morpholinyl, thiomorpholinyl, dioxacyclohexyl, azacycloheptyl, azacyclooctyl, diazacycloheptanyl (e.g., 1,4-diazacycloheptan-1-yl) and diazacyclooctyl. Examples of bridged heterocyclyl, fused heterocyclyl and spiro-heterocyclyl groups include, but are not limited to, 6-azabicyclo[3.1.1]heptan-3-yl, 2,5-diazabicyclo[2.2.1]heptan-2-yl, 3,6-diazabicyclo[3.1.1]heptan-3-yl, 3-azabicyclo[3.2.1]octan-8-yl, 3,8-diazabicyclo[3.2.1]octan-8-yl, 3,8-diazabicyclo[3.2.1]octan-3-yl, 2,5-diazabicyclo[2.2.2]octan-2-yl, octahydro-1H-indolyl, pyrrolidinocyclopropyl, cyclopentylaziridinyl, pyrrolidinocyclobutyl, pyrrolidinopyrrolidinyl (e.g., hexahydropyrrolo[3,4-c]pyrrolyl), pyrrolidinopiperidinyl, pyrrolidinopiperazinyl, pyrrolidinomorpholinyl, piperidinomorpholinyl, and azaspirocycloalkyl (e.g., 5-to 20-membered azaspirocycloalkyl, such as 3-azaspiro[5.5]undecan-3-yl). The heterocyclyl may be unsubstituted or substituted as explicitly defined (e.g., mono-, di-, tri-, or poly-substituted) by a substituent(s) optionally selected from the group consisting of deuterium, hydroxy, amino, mercapto, nitro, halogen, cyano, optionally deuterated C₁₋₆ alkyl, halogenated C₁₋₆ alkyl, optionally deuterated C₃₋₆ cycloalkyl, optionally deuterated C₁₋₆ alkoxy, optionally deuterated C₁₋₆ alkyl-NH—, NH₂-C₁₋₆ alkylene, optionally deuterated C₁₋₆ alkyl-NHC(O)—, optionally deuterated C₁₋₆ alkyl-C(O)NH— and any combination thereof.

As used herein, the term “nitrogen-containing monocyclic heterocyclyl”, used alone or in combination, refers to 4-to 20-membered (e.g., 4-to 15-membered, 4-to 14-membered, 4-to 12-membered, 4-to 11-membered, 4-to 10-membered, 4-to 9-membered, 4-to 8-membered, 4-to 7-membered, 4-to 6-membered, 4-to 5-membered, or 5-to 9-membered) saturated or partially unsaturated (i.e., containing one or more double bonds, but not having a fully conjugated π-electron system) monocyclic monovalent cyclic hydrocarbon group containing one nitrogen atom and optionally one or more (e.g., from 1 to 5, or from 1 to 4, from 1 to 3, from 1 to 2, or 1) heteroatoms independently selected from sulfur, oxygen, and nitrogen. Representative examples of the nitrogen-containing monocyclic heterocyclyl include, but are not limited to, azetidinyl, pyrrolidinyl, imidazolidinyl, pyrazolidyl, oxazolidinyl, thiazolidinyl, piperidinyl, piperazinyl, morpholinyl, thiomorpholinyl, azacycloheptyl, azacyclooctyl, diazacycloheptanyl (e.g., 1,4-diazacycloheptan-1-yl), and diazacyclooctyl. The nitrogen-containing monocyclic heterocyclyl may be unsubstituted or substituted as explicitly defined (e.g., mono-, di-, tri-, or poly-substituted) by a substituent(s) optionally selected from the group consisting of deuterium, hydroxyl, amino, mercapto, nitro, halogen, cyano, optionally deuterated C₁₋₆ alkyl, halogenated C₁₋₆ alkyl, optionally deuterated C₃₋₆ cycloalkyl, optionally deuterated C₁₋₆ alkoxy, optionally deuterated C₁₋₆ alkyl-NH—, NH₂-C₁₋₆ alkylene, optionally deuterated C₁₋₆ alkyl-NHC(O)—, optionally deuterated C₁₋₆ alkyl-C(O)NH— or any combination thereof.

As used herein, the term “nitrogen-containing bridged heterocyclyl”, used alone or in combination, refers to 5-to 20-membered (e.g., 5-to 15-membered, 5-to 14-membered, 6-to 12-membered, 6-to 11-membered, 6-to 10-membered, 5-to 9-membered, 5-to 8-membered, 5-to 7-membered, 5-to 6-membered, or 6-to 9-membered) saturated or partially unsaturated (i.e., containing one or more double bonds, but not having a fully conjugated π-electron system) bicyclic, tricyclic or polycyclic monovalent cyclic bridged hydrocarbon group containing one nitrogen atom and optionally one or more (e.g., from 1 to 5, or from 1 to 4, from 1 to 3, from 1 to 2, or 1) heteroatoms independently selected from sulfur, oxygen, and nitrogen. Examples of nitrogen-containing bridged heterocyclyl include, but are not limited to, 6-azabicyclo[3.1.1]heptan-3-yl, 2,5-diazabicyclo[2.2.1]heptan-2-yl, 3,6-diazabicyclo[3.1.1]heptan-3-yl, 3-azabicyclo[3.2.1]octan-8-yl, 3,8-diazabicyclo[3.2.1]octan-8-yl, 3,8-diazabicyclo[3.2.1]octan-3-yl, and 2,5-diazabicyclo[2.2.2]octan-2-yl. The nitrogen-containing bridged heterocyclyl may be unsubstituted or substituted as explicitly defined (e.g., mono-, di-, tri-, or poly-substituted) by a substituent(s) optionally selected from the group consisting of deuterium, hydroxyl, amino, mercapto, nitro, halogen, cyano, optionally deuterated C₁₋₆ alkyl, halogenated C₁₋₆ alkyl, optionally deuterated C₃₋₆ cycloalkyl, optionally deuterated C₁₋₆ alkoxy, optionally deuterated C₁₋₆ alkyl-NH—, NH₂-C₁₋₆ alkylene, optionally deuterated C₁₋₆ alkyl-NHC(O)—, optionally deuterated C₁₋₆ alkyl-C(O)NH— or any combination thereof.

As used herein, the term “—O-optionally substituted heterocyclyl”, used alone or in combination, refers to a group formed by optionally substituted heterocyclyl connected to oxygen. The definition of “heterocyclyl” may be as defined above. Examples of the term “heterocyclyl” include, but are not limited to, monocyclic heterocyclyl (e.g., 4-to 20-membered monocyclic heterocyclyl), bridged heterocyclyl (e.g., 5-to 20-membered bridged heterocyclyl or 7-to 15-membered bridged heterocyclyl), fused heterocyclyl, and spiro heterocyclyl (e.g., 5-to 20-membered spiro heterocyclyl). Optionally, the heterocyclyl in the term “—O-optionally substituted heterocyclyl” refers to a 4-to 20-membered mono-, bi-, tri-, or polycyclic heterocyclyl containing one or more (e.g., 1-4, or 1, 2, 3, or 4) heteroatoms selected from nitrogen, oxygen, and sulfur. Examples of the “heterocyclyl” include, but are not limited to, azetidinyl, oxetanyl, pyrrolidinyl, imidazolidinyl, pyrazolidyl, tetrahydrofuranyl, tetrahydropyranyl, tetrahydrothienyl, tetrahydrothiopyranyl, oxazolidinyl, thiazolidinyl, piperidinyl, piperazinyl, morpholinyl, thiomorpholinyl, dioxacyclohexyl, azacycloheptyl, diazacycloheptanyl (e.g., 1,4-diazacycloheptan-1-yl), azacyclooctyl, diazacyclooctyl, azabicyclo[3.1.1]heptanyl, azabicyclo[2.2.1]heptanyl, azabicyclo[3.2.1]octanyl, azabicyclo[2.2.2]octanyl, diazabicyclo[3.1.1]heptanyl, diazabicyclo[2.2.1]heptanyl, diazabicyclo[3.2.1]octanyl (e.g., 3,8-diazabicyclo[3.2.1]octan-3-yl), and diazabicyclo[2.2.2]octanyl (e.g., 2,5-diazabicyclo[2.2.2]octan-2-yl). As used herein, the heterocyclyl is optionally substituted with a substituent selected from deuterium, hydroxy, amino, mercapto, nitro, halogen, cyano, optionally deuterated C₁₋₆ alkyl, halogenated C₁₋₆ alkyl, optionally deuterated C₃₋₆ cycloalkyl, optionally deuterated C₁₋₆ alkoxy, optionally deuterated C₁₋₆ alkyl-NH—, NH₂-C₁₋₆ alkylene, optionally deuterated C₁₋₆ alkyl-NHC(O)—, optionally deuterated C₁₋₆ alkyl-C(O)NH— or any combination thereof.

As used herein, the term “heterocyclylene”, used alone or in combination, refers to a 4-to 20-membered (optionally 4-to 15-membered, 4-to 14-membered, 4-to 13-membered, 4-to 12-membered, 4-to 11-membered, 4-to 10-membered, 4-to 9-membered, 4-to 8-membered, 4-to 7-membered, 4-to 6-membered, 4-to 5-membered, or 4-to 9-membered) saturated or partially unsaturated (i.e., containing one or more double bonds, but not having a fully conjugated π-electron system) monocyclic, bicyclic, tricyclic or polycyclic bivalent cyclic hydrocarbon group containing one or more (e.g., from 1 to 5, or from 1 to 4, from 1 to 3, from 1 to 2, or 1) heteroatoms independently selected from sulfur, oxygen, and nitrogen. In some embodiments, “heterocyclylene” may refer to e.g., a 4-to 15-membered (optionally 4-to 14-membered, 4-to 13-membered, 4-to 12-membered, 4-to 11-membered, 4-to 10-membered, 4-to 9-membered, 4-to 8-membered, 4-to 7-membered, 4-to 6-membered, 4-to 5-membered, or 4-to 9-membered) saturated or partially unsaturated (i.e., containing one or more double bonds, but not having a fully conjugated π-electron system) monocyclic, bicyclic, tricyclic or polycyclic bivalent cyclic hydrocarbon group containing one or more (e.g., from 1 to 5, or from 1 to 4, from 1 to 3, from 1 to 2, or 1) heteroatoms independently selected from sulfur, oxygen, and nitrogen. Examples of the heterocyclylene groups include, but are not limited to, monocyclic heterocyclylene (e.g., 4-to 20-membered monocyclic heterocyclylene), bridged heterocyclylene (e.g., 5-to 20-membered bridged heterocyclylene, and 7-to 15-membered bridged heterocyclylene), fused heterocyclylene (e.g., 5-to 20-membered fused heterocyclylene) and spiro-heterocyclylene groups (e.g., 5-to 20-membered spiro-heterocyclylene). Representative examples of the monocyclic heterocyclylene include, but are not limited to, azetidinylene, oxetanylene, pyrrolidinylene, imidazolidinylene, pyrazolidylene, tetrahydrofuranylene, tetrahydropyranylene, tetrahydrothienylene, tetrahydrothiopyranylene, oxazolidinylene, thiazolidinylene, piperidinylene, piperazinylene, morpholinylene, thiomorpholinylene, azacycloheptylene, azacyclooctylene, dioxacyclohexylene, diazacycloheptanylene (e.g., 1,4-diazacycloheptanylene, 4,5-diazacycloheptanylene, 1,3-diazacycloheptanylene), and diazacyclooctyl. Examples of the bridged heterocyclylene, fused heterocyclylene and spiro-heterocyclylene groups include, but are not limited to, 6-azabicyclo[3.1.1]heptanylene, 2,5-diazabicyclo[2.2.1]heptanylene, 3,6-diazabicyclo[3.1.1]heptanylene, 3-azabicyclo[3.2.1]octanylene, 3,8-diazabicyclo[3.2.1]octanylene, 3,8-diazabicyclo[3.2.1]octanylene, 2,5-diazabicyclo[2.2.2]octanylene, octahydro-1H-indolylene, pyrrolidinocyclopropylene, cyclopentylaziridinylene, pyrrolidinocyclobutylene, pyrrolidinopyrrolidinylene (e.g., hexahydropyrrolo[3,4-c]pyrrolylene), pyrrolidinopiperidinylene, pyrrolidinopiperazinylene, pyrrolidinomorpholinylene, piperidinomorpholinylene, and azaspirocycloalkylene (e.g., 5-to 20-membered azaspirocycloalkylene, such as 3-azaspiro[5.5]undecanylene). The heterocyclylene may be unsubstituted or substituted as explicitly defined (e.g., mono-, di-, tri-, or poly-substituted) by a substituent(s) optionally selected from the group consisting of deuterium, hydroxy, amino, mercapto, nitro, halogen, cyano, optionally deuterated C₁₋₆ alkyl, halogenated C₁₋₆ alkyl, optionally deuterated C₃₋₆ cycloalkyl, optionally deuterated C₁₋₆ alkoxy, optionally deuterated C₁₋₆ alkyl-NH—, NH₂-C₁₋₆ alkylene, optionally deuterated C₁₋₆ alkyl-NHC(O)—, optionally deuterated C₁₋₆ alkyl-C(O)NH— or any combination thereof.

As used herein, the term “nitrogen-containing monocyclic heterocyclylene”, used alone or in combination, refers to 4-to 20-membered (e.g., 4-to 15-membered, 4-to 14-membered, 4-to 12-membered, 4-to 11-membered, 4-to 10-membered, 4-to 9-membered, 4-to 8-membered, 4-to 7-membered, 4-to 6-membered, 4-to 5-membered, or 5-to 9-membered) saturated or partially unsaturated (i.e., containing one or more double bonds, but not having a fully conjugated π-electron system) monocyclic bivalent cyclic hydrocarbon group containing one nitrogen atom and optionally one or more (e.g., from 1 to 5, or from 1 to 4, from 1 to 3, from 1 to 2, or 1) heteroatoms independently selected from sulfur, oxygen, and nitrogen. Representative examples of the nitrogen-containing monocyclic heterocyclylene include, but are not limited to, piperidinylene, piperazinylene, morpholinylene, azetidinylene, pyrrolidinylene, imidazolidinylene, pyrazolidylene, oxazolidinylene, thiazolidinylene, thiomorpholinylene, azacycloheptylene, diazacycloheptanylene, azacyclooctylene, and diazacyclooctylene. The nitrogen-containing monocyclic heterocyclylene may be unsubstituted or substituted as explicitly defined (e.g., mono-, di-, tri-, or poly-substituted) by a substituent(s) optionally selected from the group consisting of deuterium, hydroxy, amino, mercapto, nitro, halogen, cyano, optionally deuterated C₁₋₆ alkyl, halogenated C₁₋₆ alkyl, optionally deuterated C₃₋₆ cycloalkyl, optionally deuterated C₁₋₆ alkoxy, optionally deuterated C₁₋₆ alkyl-NH—, NH₂-C₁₋₆ alkylene, optionally deuterated C₁₋₆ alkyl-NHC(O)—, optionally deuterated C₁₋₆ alkyl-C(O)NH— or any combination thereof.

As used herein, the term “nitrogen-containing bridged heterocyclylene”, used alone or in combination, refers to 5-to 20-membered (e.g., 5-to 15-membered, 5-to 14-membered, 6-to 12-membered, 6-to 11-membered, 6-to 10-membered, 5-to 9-membered, 5-to 8-membered, 5-to 7-membered, 5-to 6-membered, or 6-to 9-membered) saturated or partially unsaturated (i.e., containing one or more double bonds, but not having a fully conjugated π-electron system) bicyclic, tricyclic or polycyclic bivalent bridged cyclic hydrocarbon group containing one nitrogen atom and optionally one or more (e.g., from 1 to 5, or from 1 to 4, from 1 to 3, from 1 to 2, or 1) heteroatoms independently selected from sulfur, oxygen, and nitrogen. Examples of the nitrogen-containing bridged heterocyclylene include, but are not limited to, 6-azabicyclo[3.1.1]heptanylene, 2,5-diazabicyclo[2.2.1]heptanylene, 3,6-diazabicyclo[3.1.1]heptanylene, 3-azabicyclo[3.2.1]octanylene, 3,8-diazabicyclo[3.2.1]octanylene, 3,8-diazabicyclo[3.2.1]octanylene, and 2,5-diazabicyclo[2.2.2]octanylene. The nitrogen-containing heterocyclylene may be unsubstituted or substituted as explicitly defined (e.g., mono-, di-, tri-, or poly-substituted) by a substituent(s) optionally selected from the group consisting of deuterium, hydroxyl, amino, mercapto, nitro, halogen, cyano, optionally deuterated C₁₋₆ alkyl, halogenated C₁₋₆ alkyl, optionally deuterated C₃₋₆ cycloalkyl, optionally deuterated C₁₋₆ alkoxy, optionally deuterated C₁₋₆ alkyl-NH—, NH₂-C₁₋₆ alkylene, optionally deuterated C₁₋₆ alkyl-NHC(O)—, optionally deuterated C₁₋₆ alkyl-C(O)NH— or any combination thereof.

As used herein, the term “alkynylene”, used alone or in combination, refers to a linear or branched bivalent hydrocarbon group containing from 2 to 8 (e.g., from 2 to 6, from 2 to 5, from 2 to 4, or preferably 2) carbon atoms and having one or more (e.g., from 1 to 3, from 1 to 2, or 1) carbon-carbon triple bonds. Examples of alkynylene include, but are not limited to, ethynylene, 1-propynylene, 1-butynylene, and 1,3-diynylene.

As used herein, the term “alkynyl”, used alone or in combination, refers to a linear or branched monovalent hydrocarbon group containing from 2 to 8 (e.g., from 2 to 6, from 2 to 5, from 2 to 4, or preferably 2) carbon atoms and having one or more (e.g., from 1 to 3, from 1 to 2, or 1) carbon-carbon triple bonds. Examples of C₂₋₆ alkynyl include, but are not limited to, ethynyl, 1-propynyl, 1-butynyl, and 1,3-diynyl.

As used herein, the term “alkenylene”, used alone or in combination, refers to a linear or branched divalent hydrocarbon group containing from 2 to 8 (e.g., from 2 to 6, from 2 to 5, from 2 to 4, from 2 to 3, or 2) carbon atoms and having one or more (e.g., from 1 to 3, from 1 to 2, or 1) carbon-carbon double bonds. Examples of alkenylene groups include, but are not limited to, vinylene (e.g., —CH—CH—), 1-propenylene, allylidene, 1-butenylene, 2-butenylene, 3-butenylene, isobutenylene, pentenylene, n-pent-2,4-dienylene, 1-methyl-but-1-enylene, 2-methyl-but-1-enylene, 3-methyl-but-1-enylene, 1-methyl-but-2-enylene, 2-methyl-but-2-enylene, 3-methyl-but-2-enylene, 1-methyl-but-3-enylene, 2-methyl-but-3-enylene, 3-methyl-but-3-enylene, and hexenylene.

As used herein, the term “alkenyl”, used alone or in combination, refers to a linear or branched monovalent hydrocarbon group containing from 2 to 8 (e.g., from 2 to 6, from 2 to 5, from 2 to 4, from 2 to 3, or 2) carbon atoms and having one or more (e.g., from 1 to 3, from 1 to 2, or 1) carbon-carbon double bonds. Examples of C₂₋₆ alkenyl group include, but are not limited to, vinyl (e.g., CH₂—CH—), 1-propenyl, allyl, 1-butenyl, 2-butenyl, 3-butenyl, isobutenyl, pentenyl, n-pent-2,4-dienyl, 1-methyl-but-1-enyl, 2-methyl-but-1-enyl, 3-methyl-but-1-enyl, 1-methyl-but-2-enyl, 2-methyl-but-2-enyl, 3-methyl-but-2-enyl, 1-methyl-but-3-enyl, 2-methyl-but-3-enyl, 3-methyl-but-3-enyl, and hexenyl.

As used herein, “bornylane” or “bornane” (also known as 1,7,7-trimethylbicyclo[2.2.1]heptane; camphane; bornylane) has a definition known to those skilled in the art. As used herein, “camphanyl” or “bornyl” refers to a monovalent group of bornane, i.e., the group remaining after any one of the hydrogens in bornane is removed. Representative examples of “bornyl” include, but are not limited to, 1,7,7-trimethylbicyclo[2.2.1]heptan-2-yl, 1,7,7-trimethylbicyclo[2.2.1]heptan-3-yl, 1,7,7-trimethylbicyclo[2.2.1]heptan-4-yl, 1,7,7-trimethylbicyclo[2.2.1]heptan-5-yl, 1,7,7-trimethylbicyclo[2.2.1]heptan-6-yl,

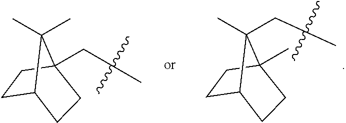 [SMILES: *CC12CCC(CC1)C2(C)C ; *CC1(C)C2CCC1(C)CC2]

As used herein, “bicyclo[2.2.1]heptane” also known as “norbornane”, has a definition known to those skilled in the art. As used herein, “bicyclo[2.2.1]heptyl” or “norbornyl” refers to a monovalent group of bicyclo[2.2.1]heptane, i.e., the group remaining after any hydrogen in bicyclo[2.2.1]heptane is removed. Representative examples of “bicyclo[2.2.1]heptyl” include, but are not limited to, bicyclo[2.2.1]heptan-2-yl, bicyclo[2.2.1]heptan-3-yl, bicyclo[2.2.1]heptan-4-yl, bicyclo[2.2.1]heptan-5-yl, or bicyclo[2.2.1]heptan-6-yl.

As used herein, the term “bicyclo[2.2.1]heptene” has a definition known to those skilled in the art. As used herein, “bicyclo[2.2.1]heptenyl” refers to a monovalent group of bicyclo[2.2.1]heptene, i.e., the group remaining after any hydrogen in bicyclo[2.2.1]heptene is removed. Representative examples of “bicyclo[2.2.1]heptenyl” include, but are not limited to, bicyclo[2.2.1]hept-5-en-2-yl, bicyclo[2.2.1]hept-5-en-3-yl, or bicyclo[2.2.1]hept-5-en-7-yl.

As used herein, “adamantane” (also known as tricyclo[3.3.1.1^{3,7}]decane) has a definition known to those skilled in the art, and its structural formula is e.g., as follows:

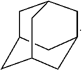

As used herein, “adamantanyl” refers to a monovalent group of adamantane, that is, the group remaining after any hydrogen in adamantane is removed. Representative examples of “adamantanyl” include, but are not limited to, 1-adamantanyl, 2-adamantanyl, 3-adamantanyl, 4-adamantanyl, 5-adamantanyl, 6-adamantanyl, 7-adamantanyl, 8-adamantanyl, 9-adam antanyl, or 10-adamantanyl.

As used herein, “noradamantane” has a definition known to those skilled in the art, and its structural formula is e.g., as follows:

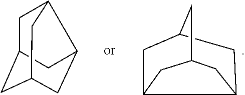 [SMILES: C1C2CC3CC1CC3C2 ; C1C2CC3CC1CC3C2]

As used herein, “noradamantanyl” refers to a monovalent group of noradamantane, that is, the group remaining after any hydrogen in noradamantane is removed. Representative examples of “noradamantanyl” include, but are not limited to, 1-noradamantanyl, 2-noradamantanyl, 3-noradamantanyl, 4-noradamantanyl, 5-noradamantanyl, 6-noradamantanyl, 7-noradamantanyl, 8-noradamantanyl or 9-noradamantanyl.

As used herein, “adamantanamine” has the definitions known to those skilled in the art, namely referring to an adamantane having an amino substituent, wherein the amino substituent can replace a hydrogen on a carbon at any position in the adamantane. An example of “adamantanamine” can be adamantan-1-amine (corresponding English chemical name is adamantan-1-amine or Tricyclo[3.3.1.1^{3,7}]decan-1-amine; CAS No.: 768-94-5), with the following structural Formula:

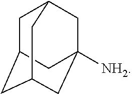 [SMILES: NC12CC3CC(CC(C3)C1)C2]

In the present disclosure, the term “leaving group” used alone or in combination is well known to those skilled in the art, which is a leaving molecular fragment (ion or neutral molecule) that carries a pair of electrons from a reactant in chemical reactions, as is a term used in nucleophilic substitution and elimination reactions. Common ionic leaving groups include Cl⁻, Br⁻, I⁻ and sulfonate (such as p-toluenesulfonate, TsO⁻), and neutral molecular leaving groups include water, ammonia and alcohol. In the present disclosure, those skilled in the art can select an appropriate leaving group as needed, such as but not limited to —N₃, halogen, methanesulfonyloxy (MsO—), trifluoromethanesulfonyloxy (TfO—) or p-toluenesulfonyloxy (TsO—), etc.

Salts or pharmaceutically acceptable salts, enantiomers, stereoisomers, solvates, polymorphs of the compounds of Formula (I) or Formula (II) of the present disclosure are also encompassed within the scope of the present invention.

In all embodiments of the present disclosure, the salts or pharmaceutically acceptable salts of the compounds of Formula (I) or Formula (II) refer to non-toxic inorganic or organic acid and/or base addition salts. Examples include: sulfates, hydrohalides (including hydrochlorides and hydrobromates), maleates, sulfonates, citrates, lactates, lactobionates, L-tartrates, fumarates, L-malates, L-lactates, α-Ketoglutarates, hippurates, D-glucuronates, D-gluconates, α-D-glucoheptonates, glycolates, mucates, L-ascorbates, orotates, picrates, glycinates, alaninates, argininates, cinnamates, laurates, pamoates, sebacates, benzenesulfonates, methanesulfonates, ethanesulfonates, edisylates, formates, acetates, 2,2-dichloroacetates, trimethylacetates, propionates, valerates, palmitates, triphenylacetates, 2-ethylsuccinates, iodates, niacinates, L-pyroglutamates, L-prolinates, ferulates, 2-hydroxyethanesulfonates, nitrates, gentisates, cholates, salicylates, terephthalates, glutarates, adipates, stearates, oleates, undecenoates, camphorates, camphorsulfonates, dodecyl sulfonates, phosphates, thiocyanates, dihydrophosphates, pyrophosphates, metaphosphates, oxalates, carbonates, malonates, benzoates, mandelates, succinates, pyruvates, para-chlorobenzenesulfonates, 1,5-naphthalenedisulfonates, 3-hydroxy-2-naphthoates, 1-hydroxy-2-naphthoates, 2-naphthalenesulfonates, hydroxyacetates, or p-toluenesulfonates, etc.

“Pharmaceutically acceptable carrier” refers to a pharmaceutically acceptable material, such as a filler, stabilizer, dispersant, suspending agent, diluent, excipient, thickener, solvent, or encapsulating material, with which the useful compounds according to the present disclosure are carried or transported into or administered to a patient so that they can perform their intended function. Generally, such constructs are carried or transported from one organ or part of the body to another organ or part of the body. The carrier is compatible with the other ingredients of the formulation, including the compounds useful in the present disclosure, and is not harmful to the patient, and the carrier must be “acceptable”. Some examples of materials that can be used as pharmaceutically acceptable carriers include sugars such as lactose, glucose, and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository wax; oils such as peanut oil, cottonseed oil, safflower oil, sesame oil, olive oil, corn oil and soybean oil; glycols such as propylene glycol; polyols such as glycerol, sorbitol, mannitol, and polyethylene glycol; esters such as ethyl oleate and ethyl laurate; agar; buffers such as magnesium hydroxide and aluminum hydroxide; surfactant phosphate buffer solution; and other common non-toxic compatible substances used in pharmaceutical formulations.

As used herein, the term “room temperature” refers to the ambient temperature, such as 20-30 °C.

As used herein, “stereoisomer” refers to a compound with the same chemical structural formula, but a different arrangement of atoms or groups in space. Stereoisomers include enantiomers, diastereomers, conformational isomers (rotational isomers), geometric isomers (cis/trans isomers), atropisomers, and so on.

As used herein, the term “solvate” refers to an association or complex formed by the interaction between one or more solvent molecules and the compounds of the present invention. Examples of solvents include water, isopropanol, ethanol, methanol, DMSO, ethyl acetate, acetic acid and ethanolamine. The term “hydrate” means that a complex formed with water.

As used herein, the term “chiral” refers to a molecule that is nonsuperimposable on its mirror image; whereas “achiral” refers to a molecule that can be superimposed on its mirror image.

As used herein, the term “enantiomers” refers to two isomers of a compound that are non-superimposable mirror images.

As used herein, the term “diastereomers” refers to stereoisomers that have two or more chiral centers but are not mirror images of each other. The diastereomers have different physical properties, such as melting point, boiling point, spectral properties and reactivity. The diastereomeric mixture can be separated by high-resolution analytical operations such as electrophoresis and chromatography, such as HPLC.

### EXAMPLES

In the following description, numerous specific details are set forth in order to provide a thorough understanding of the present disclosure. The present disclosure may be practiced without some or all of these specific details. In other cases, well-known process operations have not been described in detail in order not to unnecessarily obscure the present disclosure. Although the present disclosure will be described in conjunction with specific embodiments, it should be understood that this is not intended to limit the present disclosure to these embodiments.

The following abbreviations are used throughout the specification and examples:
ACN acetonitrile
AcOH acetic acid
AcOK potassium acetate
AIBN 2,2′-azobis(2-methylpropionitrile)
Bn Benzyl
Boc t-Butyloxy carbonyl
Brettphos Pd G3 Methanesulfonato (2-dicyclohexylphosphino-3,6-dimethoxy-2′,4′,6′-tri-i-propyl-1,1′-biphenyl)(2′-amino-1,1′-biphenyl-2-yl) palladium (II)
Brettphos Dicyclohexyl[3,6-dimethoxy-2′,4′,6′-tris(1-methylethyl) [1,1′-biphenyl]-2-yl]phosphine
BINAP 2,2′-Bis(diphenylphosphino)-1,1′-binaphthalene
BPO Dibenzoyl peroxide
CataCXium A Pd G3 Methanesulfonatobutyl-bis(1-adamantyl)phosphinepalladium (II)
Cbz Carbobenzoxy
Con. Concentration
DCE 1,2-Dichloroethane
DCM dichloromethane
DEA Diethylamine
DIAD Diisopropyl azodicarboxylate
DIEA, or DIPEA N, N-diisopropylethylamine
DMA N,N-Dimethylacetamide
DMAP 4-Dimethylaminopyridine
DME Dimethylether
DMEDA N,N′-dimethylethylenediamine
DMF N,N-dimethylformamide
DMSO dimethyl sulfoxide
EDCI 1-(3-dimethylaminopropyl)-3-ethylcarbodiimide hydrochloride
ESI electrospray ionization
equiv equivalent
EtOH ethanol
EtOAc, or EA Ethyl acetate
HATU 2-(7-Azabenzotriazol-1-yl)-N,N,N′,N′-tetramethyluronium hexafluorophosphate
HPLC high performance liquid chromatography
HRMS high resolution mass spectrometry
IPA Isopropyl alcohol
LC-MS liquid chromatography-mass spectrometry
LiHMDS Lithium hexamethyldisilazide
LRMS low resolution mass spectrometry
LC liquid chromatography
Me methyl
MeCN acetonitrile
MeOH Methanol
MS mass spectrometry
MsO-Methanesulfonyloxy
NaHMDS Sodium bis(trimethylsilyl)amide
NBS N-Bromosuccinimide
NMI N-Methylimidazole
¹H NMR Proton nuclear magnetic resonance
HFIP Hexafluoroisopropanol
MeO-Methoxy
o/n Overnight
PCC Pyridinium chlorochromate
PE petroleum ether
Pin₂B₂ pinacolborane
PyBOP Benzotriazol-1-yl-oxy-tris(pyrrolidino)phosphonium hexafluorophosphate
Ruphos Pd G3 Methanesulfonato (2-dicyclohexylphosphino-2′,6′-diisopropoxy-1,1′-biphenyl)(2′-amino-1,1′-biphenyl-2-yl) palladium (II)(CAS No.: 1445085-77-7)
rt room temperature
tBu tert-butyl
tBuONa Sodium tert-butoxide
TBSCI tert-Butyldimethylchlorosilane
TCFH N,N,N′,N′-Tetramethylchloroformamidinium hexafluorophosphate
TEA triethylamine
TFA trifluoroacetic acid
T_{f}O-Trifluoromethanesulfonate
Tf₂O Triflic anhydride
THF Tetrahydrofuran
TLC thin layer chromatography
TMS tetramethylsilane
TsCl p-toluenesulfonyl chloride
TsO-Tosyloxy
TsOH p-Toluenesulfonic acid
®T₃P propylphosphonic anhydride
TTIP titanium (IV) isopropoxide
Xantphos 4,5-bis(diphenylphosphino)-9,9-dimethylxanthene
Xphos 2-dicyclohexylphosphino-2′,4′,6′-triisopropylbiphenyl

In the present disclosure, the ¹H NMR spectrum was recorded on a Bruker-500 MHz nuclear magnetic resonance instrument, by using, as a solvent, CD₃OD (δ=3.31 ppm) containing 0.1% TMS (as an internal standard); or using, as a solvent, CDCl₃ (δ=7.26 ppm) containing 0.1% TMS (as an internal standard); or using, as a solvent, DMSO-d₆ (δ=2.50 ppm) containing 0.03% TMS (as an internal standard). LC-MS spectra were recorded on a Sciex API 2000 mass spectrometer equipped with an Agilent 1100 binary pump, DAD and ELSD detectors, or on an Agilent 1260-6125B single quadrupole LC-MS system equipped with an Agilent 1260 quaternary pump, DAD and ELSD detectors. HPLC preparative purification was performed using a SHIMADZU LC-20AP system. HPLC purity was determined using either a SHIMADZU LC-30AP or Waters 1525 system. All reactions were carried out under ambient atmosphere unless otherwise specified. Reaction progress was monitored by TLC or LC-MS.

Solvents and reagents are processed as follows:
the solvents used in the reactions such as DCM, DMF, NMP, anhydrous EtOH, and anhydrous MeOH were commercially available;
preparative HPLC was performed using HPLC-grade CH₃CN and deionized water;
all other reactants, reagents and chemicals were commercially available, unless otherwise specified, or were synthesized using methods known in the art.

Unless otherwise specified, the materials and reagents used in the following examples are commercially available and used directly, or can be synthesized by using methods known in the art.

### General Synthesis Methods

Compounds and/or pharmaceutically acceptable salts thereof of the present disclosure can be synthesized using commercially available raw materials by synthetic techniques known in the art. The synthetic schemes described below illustrate the preparation of most compounds. The starting materials or reagents used in each scheme can be purchased from commercial sources or prepared by methods known to those skilled in the art. One skilled in the art can prepare the salts, racemates, enantiomers, phosphates, sulfates, hydrochlorides and prodrug forms of the compounds of Formula (I) and Formula (II) of the present disclosure according to routine techniques in the art.

### General Synthesis Method 1 for Intermediate Compounds

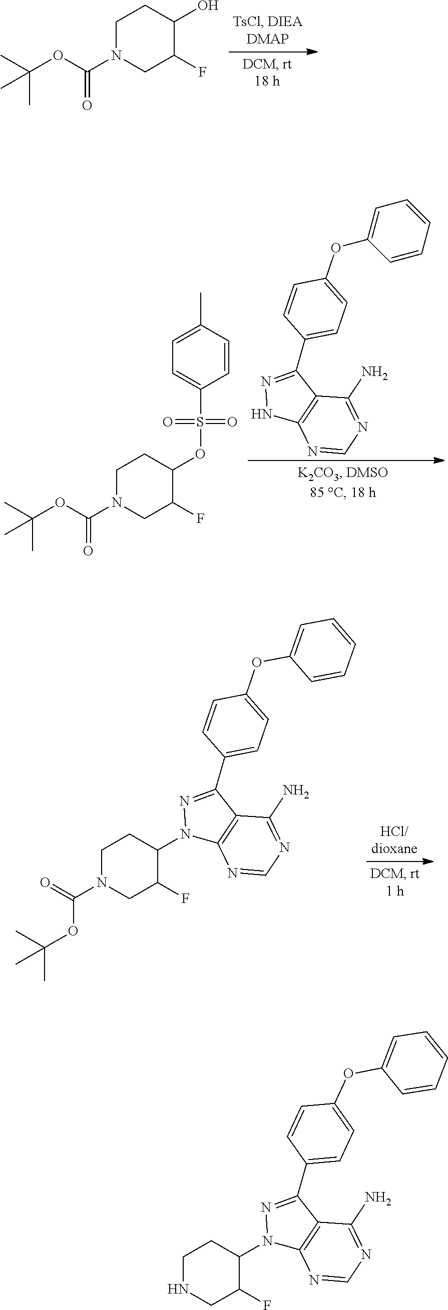

### General Synthesis Method 2 for Intermediate Compounds

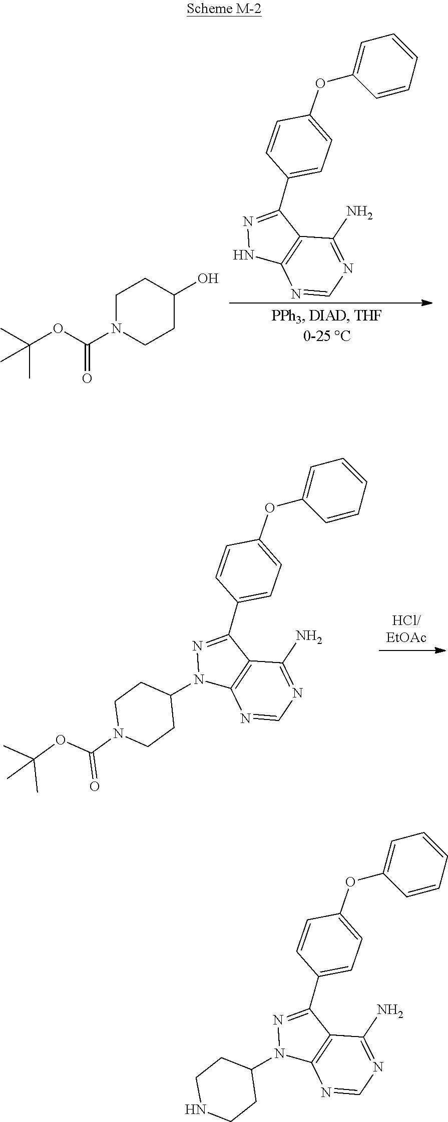 [SMILES: CC(C)(C)OC(=O)N1CCC(O)CC1 ; Nc1ncnc2[nH]nc(-c3ccc(Oc4ccccc4)cc3)c12 ; CC(C)(C)OC(=O)N1CCC(n2nc(-c3ccc(Oc4ccccc4)cc3)c3c(N)ncnc32)CC1 ; Nc1ncnc2c1c(-c1ccc(Oc3ccccc3)cc1)nn2C1CCNCC1]

### General Synthesis Method 3 for Intermediate Compounds

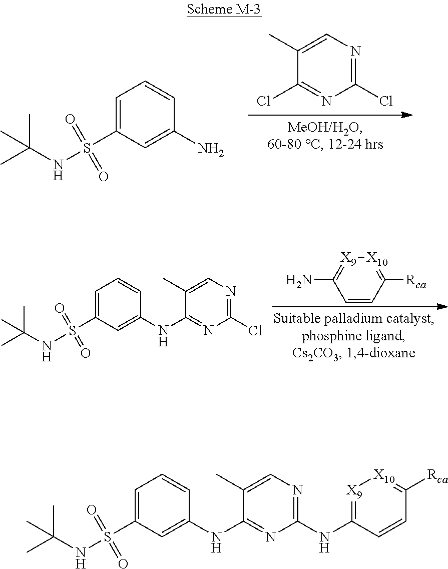

In Scheme M-3, X₉ and X₁₀ of the amine substrate used in Step 2 are the same or different and each independently represent N or CH. R_{ca} is a monovalent group corresponding to the divalent group R_{c} of the compound of Formula (I). For example, R_{ca} represents —OH, —NH(R_{d}), —C(O)NH₂, —N(R_{d})—R_{f}—NH(Rₑ), —R_{f}—C(O)NH₂, —R_{f}—C(O)OH, —C(O)OH, —R_{f}—H, —R_{f}—NH(R_{d}), —O—R_{f}—NH(R_{d}),

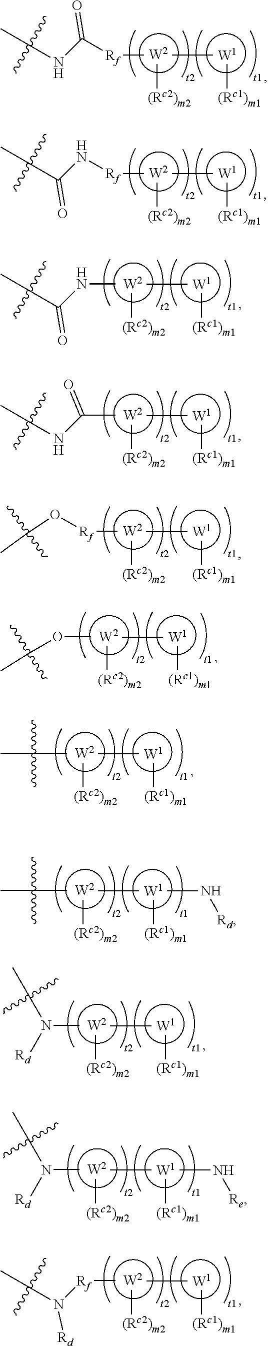

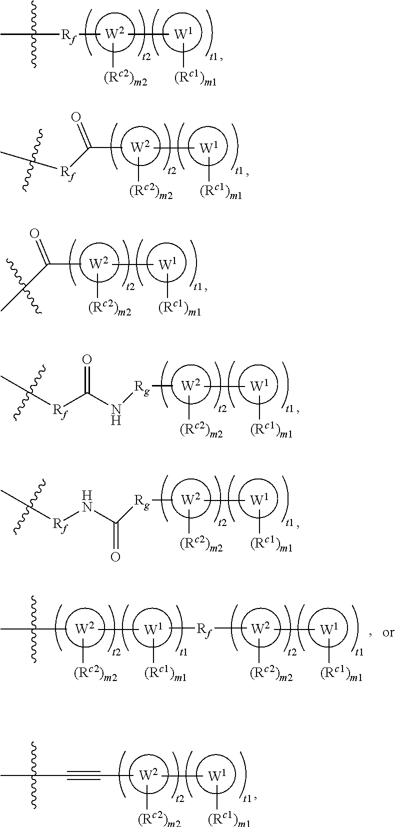

wherein R_{d}, Rₑ, R_{f}, R_{g}, R^{c1}, R^{c2}, ring W¹, ring W², t1, t2, m1, and m2 are as defined in the compound of Formula (I) and its various embodiments in the present disclosure. It should be understood that when the group R_{ca} contains reactive hydrogen that can participate in the reaction of Step 2, the reactive hydrogen can be protected by techniques and methods known to those skilled in the art, such as using protecting groups. For example, when R_{ca} represents a piperidinyl or piperazinyl group, conventional protecting groups such as Boc can be used to protect the hydrogen on the N. The removal of the protecting group can be achieved by techniques and methods known to those skilled in the art. For instance, the removal of the Boc protecting group can be accomplished under acidic conditions such as hydrochloric acid or trifluoroacetic acid.

The palladium catalyst and phosphine ligand used in Step 2 of Scheme M-3 can be conventional ones suitable for coupling reactions. There are many types of conventional palladium catalysts used for coupling reactions, such as palladium acetate (Pd(OAc)₂), tris(dibenzylideneacetone) dipalladium (Pd₂(dba)₃), diphenylphosphinoferrocene palladium dichloride, tetraphenylphosphine palladium, dichlorobis(triphenylphosphine) palladium, palladium on carbon, and so on. The phosphine ligand can be a conventional one like Xantphos or BINAP.

In Step 1 of Scheme M-3, to a solution of 3-amino-N-(tert-butyl)benzenesulfonamide (9.50 g, 41.608 mmol) in MeOH/H₂O (400 mL) was added 2,4-dichloro-5-methylpyrimidine (6.78 g, 41.608 mmol), and the reaction was stirred at 80 °C for 18 hours. After monitoring the reaction via LCMS and confirming its completion, the reaction mixture was cooled to room temperature, resulting in the precipitation of a large amount of white solid. The reaction mixture was filtered, and the filter cake was washed with methanol/water (50 mL, 1:1), dried under high vacuum at 50 °C, yielding a compound N-(tert-butyl)-3-((2-chloro-5-methylpyrimidin-4-yl)amino)benzenesulfonamide as white solid (8.00 g, 22.544 mmol, yield 54.18%). LCMS (ESI): calcd for (M) 354.09, found, (M+H)⁺ 355.10.

In Step 2 of Scheme M-3, to a solution of N-(tert-butyl)-3-((2-chloro-5-methylpyrimidin-4-yl)amino)benzenesulfonamide (1 equiv) and the corresponding amine substrate (1.3 equiv) in dioxane (50 mL) were added Cs₂CO₃ and a suitable palladium catalyst/phosphine ligand. The reaction was stirred at 100 °C for 4 hours. The reaction mixture was diluted with ethyl acetate and saturated NaCl solution, dried over Na₂SO₄, and concentrated under vacuum to obtain the crude product. The crude product was purified by silica gel flash column chromatography (eluent (v/v): EtOAc/PE=0/1-1/4) to give the target compound.

### General Synthesis Method 4 for Intermediate Compounds

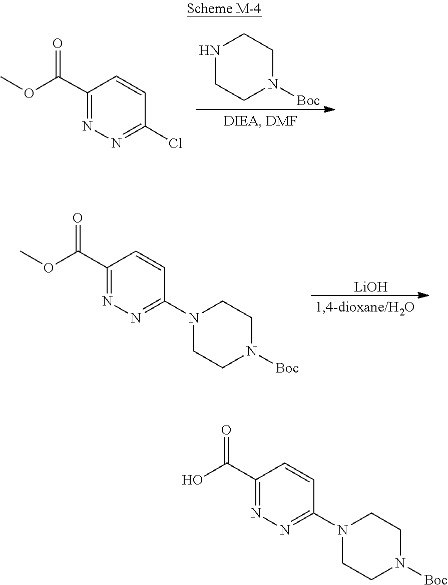 [SMILES: COC(=O)c1ccc(Cl)nn1 ; CC(C)(C)OC(=O)N1CCNCC1 ; COC(=O)c1ccc(N2CCN(C(=O)OC(C)(C)C)CC2)nn1 ; "LiOH" ; CC(C)(C)OC(=O)N1CCN(c2ccc(C(=O)O)nn2)CC1]

### General Synthesis Method 5 for Intermediate Compounds

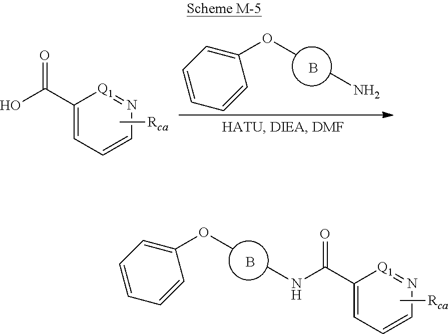

In Scheme M-5, ring B of the amine substrate and Q₁ of the acid substrate are as defined in Formula (PBM-4-1A) and its various embodiments in the present disclosure. The R_{ca} of the acid substrate used is a monovalent group corresponding to the divalent group R_{c} of the compound of Formula (I). For example, R_{ca} represents —OH, —NH(Rₐ), —C(O)NH₂, —N(Rₐ)—R_{f}—NH(Rₑ), —R_{f}—C(O)NH₂, —R_{f}—C(O)OH, —C(O)OH, —R_{f}—H, —R_{f}—NH(Rₐ), —O—R_{f}—NH(Rₐ),

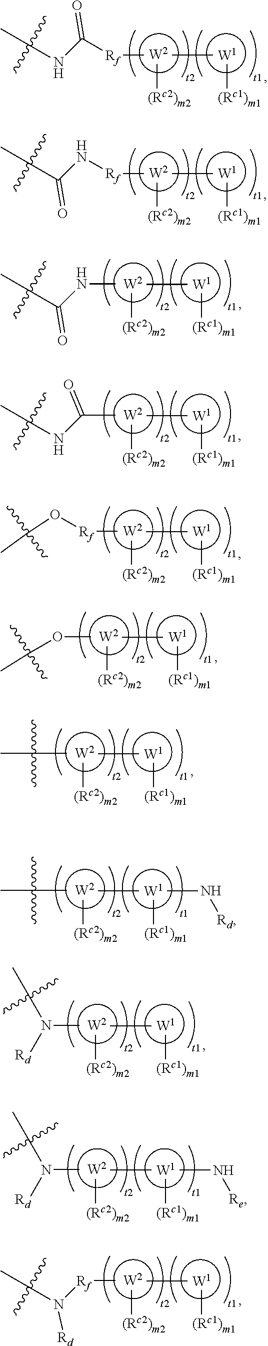

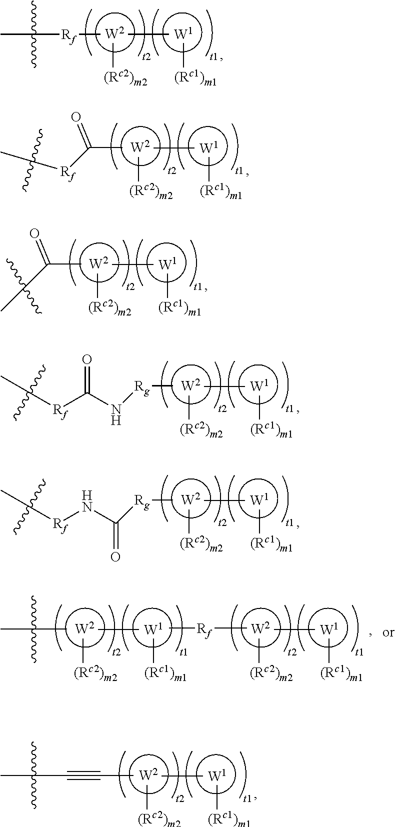

wherein R_{d}, Rₑ, R_{f}, R_{g}, R^{c1}, R^{c2}, ring W¹, ring W², t1, t2, m1, and m2 are as defined in the compound of Formula (I) and its various embodiments in the present disclosure. It should be understood that when the group R_{ca} contains an active hydrogen or reactive group that can participate in the reaction of Scheme M-5, the active hydrogen or reactive group can be protected by techniques and methods well known to those skilled in the art, such as protective groups. For example, when R_{ca} represents a piperidinyl or piperazinyl group, conventional protective groups such as Boc can be used to protect the hydrogen on N. The removal of the protective group can be achieved by techniques and methods well known to those skilled in the art, such as removing the protective group Boc under acidic conditions such as hydrochloric acid or trifluoroacetic acid.

In Scheme M-5, to a solution of the acid substrate (1 equiv) and amine substrate (1 equiv) in DMF were added HATU (1.5 equiv) and DIEA (5 equiv). The reaction mixture was then allowed to react at room temperature for 30 minutes. After monitoring the reaction via LCMS and confirming its completion, water was added to the reaction mixture, and the resulting mixture was extracted with ethyl acetate. The organic phase was washed with saturated saline and concentrated to obtain the crude product. The crude product was purified using a chromatography column (eluent (v/v): DCM/MeOH=100/1) to give the target compound.

### General Synthesis Method 6 for Intermediate Compounds

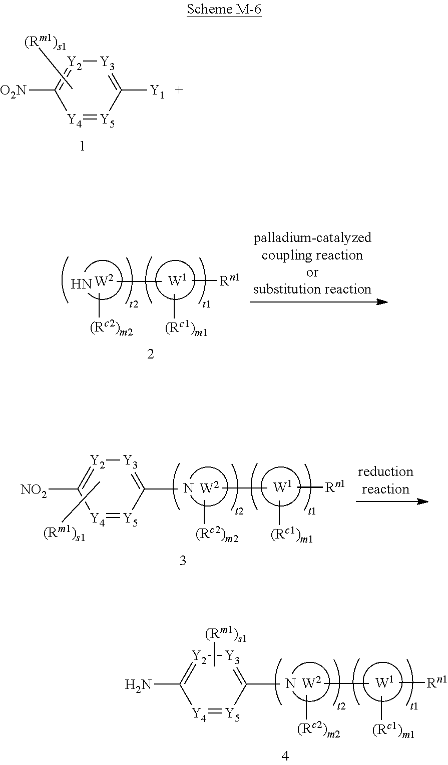

In Scheme M-6, (R^{m1})ₛ₁ of compound 1 indicates that the six-membered ring to which it is attached is optionally substituted with s1 R^{m1} groups, with each R^{m1} being the same or different and independently representing halogen, hydroxy, C₁₋₆ alkyl (such as methyl or ethyl), halo C₁₋₆ alkyl, deuterated C₁₋₆ alkyl, C₁₋₆ alkoxy (such as methoxy or isopropoxy), halo C₁₋₆ alkoxy, deuterated C₁₋₆ alkoxy, NO₂, NH₂, or cyano. s1 represents an integer of 0, 1, 2, 3, or 4. Y₁ represents halogen (such as fluorine, chlorine, or bromine), and Y₂, Y₃, Y₄, and Y₅ each independently represent N or CH, with the provision that Y₂, Y₃, Y₄, and Y₅ are not all N simultaneously. Ring W¹, ring W², t1, t2, (R^{c1})ₘ₁, and (R^{c2})ₘ₂ are as defined in the compound of Formula (I) of the present disclosure. Rⁿ¹ represents Boc, H, or —NHRₕ, where Rₕ represents hydrogen or C₁₋₆ alkyl (such as methyl or ethyl. It should be understood that when ring W¹-Rⁿ¹ contains additional reactive groups (such as —NH— or —NH₂) that can participate in the reaction of Scheme M-6, the reactive groups can be protected by techniques and methods known to those skilled in the art, such as using protecting groups.

Depending upon the target compound, the reaction substrate, reaction conditions (including reaction dosage, temperature, time, etc.), and work up, etc. in Scheme M-6 can be appropriately modified and adjusted using techniques and methods well known to those skilled in the art to obtain the desired target compound. In Scheme M-6, the substitution reaction or palladium-catalyzed coupling reaction of step 1 can be conventional techniques and methods known to those skilled in the art. When step 1 is a substitution reaction, Y₁ represents fluorine, and compounds 1 and 2 react under basic conditions (such as K₂CO₃). When step 1 is a palladium-catalyzed coupling reaction, Y₁ represents bromine, and the palladium catalyst and phosphine ligand used can be conventional ones suitable for coupling reactions. There are many types of conventional palladium catalysts for coupling reactions, such as palladium acetate (Pd(OAc)₂), tris(dibenzylideneacetone) dipalladium (Pd₂(dba)₃), diphenylphosphinoferrocene dichloropalladium, tetrakis(triphenylphosphine) palladium, dichlorobis(triphenylphosphine) palladium, palladium on carbon, etc. The phosphine ligand can be a conventional ligand such as Xantphos or BINAP. The reduction reaction of step 2 in Scheme M-6 can be conventional techniques and methods known to those skilled in the art, such as H₂/Pd/C or Fe/NH₄Cl/MeOH.

### General Synthesis Method 7 for Intermediate Compounds

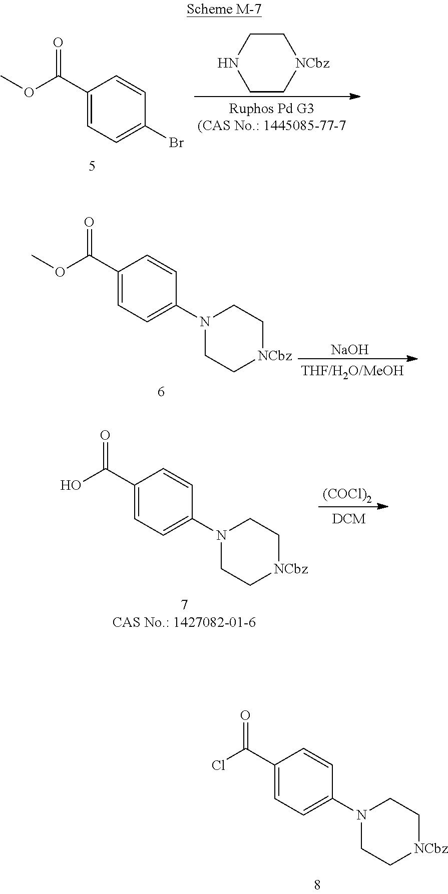

### General Synthesis Method 8 for Intermediate Compounds

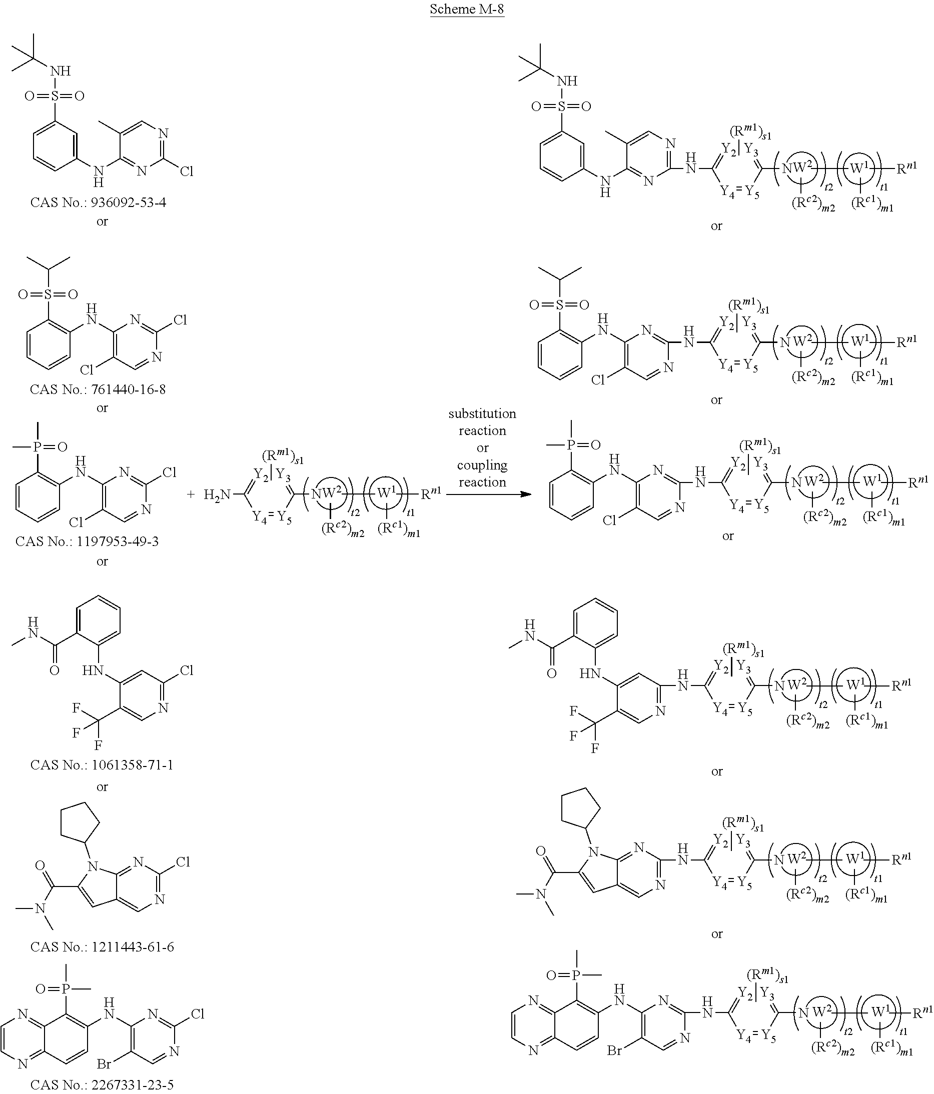

In Scheme M-8, (R^{m1})ₛ₁ of the compounds indicate that the six-membered ring to which it is attached is optionally substituted with s1 R^{m1} groups, with each R^{m1} being the same or different and independently representing halogen, hydroxy, C₁₋₆ alkyl (such as methyl or ethyl), halo C₁₋₆ alkyl, deuterated C₁₋₆ alkyl, C₁₋₆ alkoxy (such as methoxy or isopropoxy), halo C₁₋₆ alkoxy, deuterated C₁₋₆ alkoxy, NO₂, NH₂, or cyano. Y₁ represents halogen (such as fluorine, chlorine, or bromine), and Y₂, Y₃, Y₄, and Y₅ each independently represent N or CH, with the provision that Y₂, Y₃, Y₄, and Y₅ are not all N simultaneously. Ring W¹, ring W², t1, t2, (R^{c1})ₘ₁, and (R^{c2})ₘ₂ are as defined in the compound of Formula (I) of the present disclosure. Rⁿ¹ represents Boc, H, or —NHRₕ, where Rₕ represents hydrogen or C₁₋₆ alkyl (such as methyl or ethyl). It should be understood that when ring W¹-Rⁿ¹ contains additional reactive groups (such as —NH— or —NH₂) that can participate in the reaction of Scheme M-8, the reactive groups can be protected by techniques and methods known to those skilled in the art, such as using protecting groups. The removal of protecting groups can be achieved through techniques and methods known to those skilled in the art. For example, the removal of the Boc protecting group can be achieved under acidic conditions such as hydrochloric acid or trifluoroacetic acid.

Depending upon the target compound, the reaction substrate, reaction conditions (including reaction dosage, temperature, time, etc.), and work up, etc. in Scheme M-8 can be appropriately modified and adjusted using techniques and methods well known to those skilled in the art to obtain the desired target compound. In Scheme M-8, the substitution reaction or coupling reaction can be conventional techniques and methods known to those skilled in the art. For example, substitution reactions can be carried out under heating conditions in the presence of triethylamine/isopropanol, p-toluenesulfonic acid/n-butanol, DIEA/ethanol, NaHMDS, or NaI/DIEA/DMF; coupling reactions can be performed under heating conditions in the presence of Brettphos Pd G3/Brettphos ligand/Cs₂CO₃.

### General Synthesis Method 9 for Intermediate Compounds

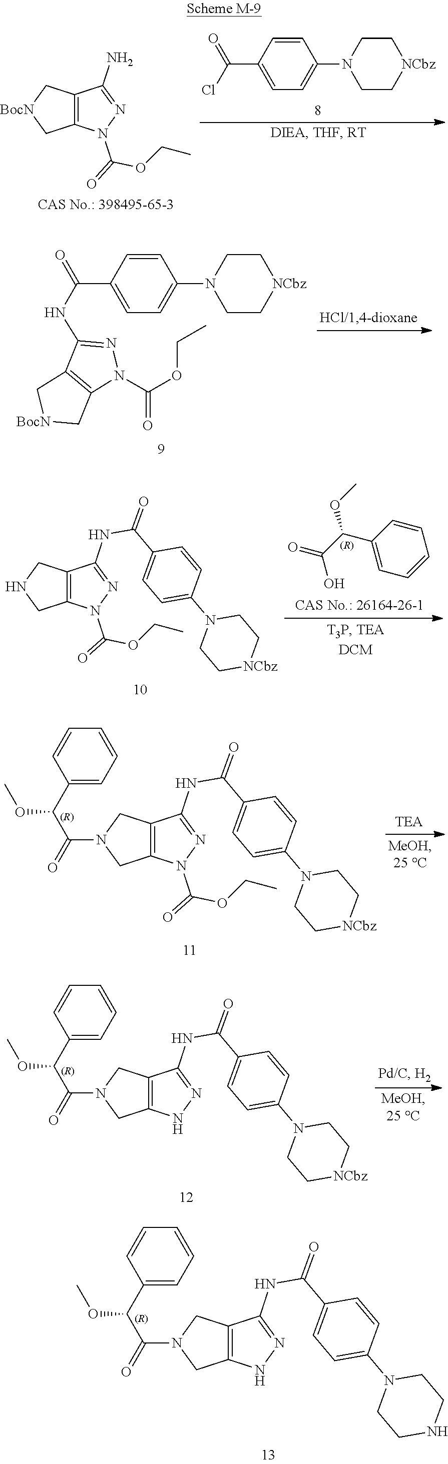

The compound 8 in Step 1 of Scheme M-9 can be prepared by referring to Scheme M-7.

### General Synthesis Method 10 for Intermediate Compounds

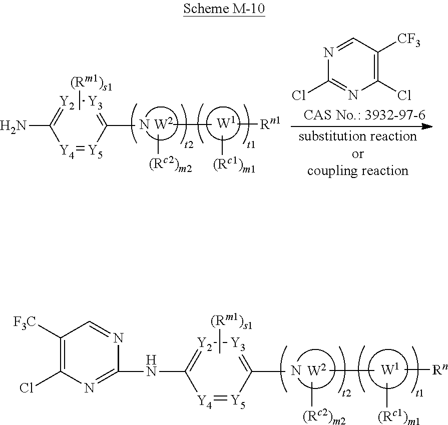

In Scheme M-10, (R^{m1})ₛ₁ of the compounds indicates that the benzene ring is optionally substituted with s1 R^{m1} groups, with each R^{m1} being the same or different and independently representing halogen, hydroxy, C₁₋₆ alkyl (such as methyl or ethyl), halo C₁₋₆ alkyl, deuterated C₁₋₆ alkyl, C₁₋₆ alkoxy (such as methoxy or isopropoxy), halo C₁₋₆ alkoxy, deuterated C₁₋₆ alkoxy, NO₂, NH₂, or cyano. s1 represents an integer of 0, 1, 2, 3, or 4. Y₁ represents halogen (such as fluorine, chlorine, or bromine), and Y₂, Y₃, Y₄, and Y₅ each independently represent N or CH, with the proviso that Y₂, Y₃, Y₄, and Y₅ are not all N at the same time. Rings W¹, W², t1, t2, (R^{c1})ₘ₁, (R^{c2})ₘ₂ are as defined in the compound of Formula (I) of the present disclosure. Rⁿ¹ represents Boc, H, or —NHRₕ, where Rₕ represents hydrogen or C₁₋₆ alkyl (such as methyl or ethyl). It should be understood that when ring W¹-Rⁿ¹ contains additional reactive groups (such as —NH— or —NH₂) that can participate in the reaction of Scheme M-10, the reactive groups can be protected by techniques and methods known to those skilled in the art, such as using protecting groups.

Depending upon the target compound, the reaction substrate, reaction conditions (including reaction dosage, temperature, time, etc.), and work up, etc. in Scheme M-10 can be appropriately modified and adjusted using techniques and methods well known to those skilled in the art to obtain the desired target compound. In Scheme M-10, the substitution reaction or coupling reaction can be conventional techniques and methods known to those skilled in the art. For example, substitution reactions can be carried out under heating conditions in the presence of triethylamine/isopropanol, DIEA/ethanol, NaHMDS, or NaI/DIEA/DMF; coupling reactions can be performed under heating conditions in the presence of Brettphos Pd G3/Brettphos ligand/Cs₂CO₃.

### General Synthesis Method 11 for Intermediate Compounds

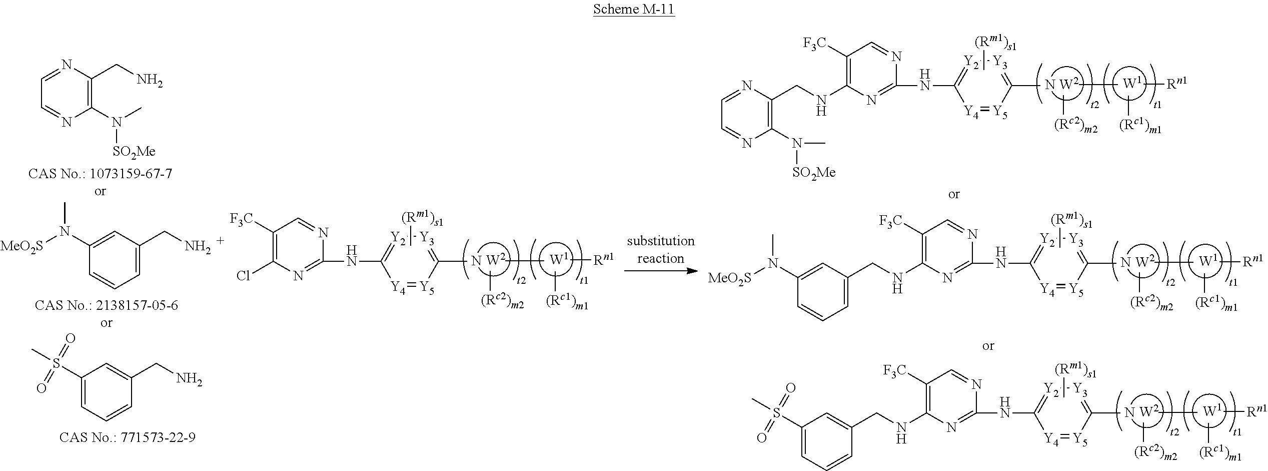

In Scheme M-11, (R^{m1})ₛ₁ of the compounds indicates that the benzene ring is optionally substituted with s1 R^{m1} groups, with each R^{m1} being the same or different and independently representing halogen, hydroxy, C₁₋₆ alkyl (such as methyl or ethyl), halo C₁₋₆ alkyl, deuterated C₁₋₆ alkyl, C₁₋₆ alkoxy (such as methoxy or isopropoxy), halo C₁₋₆ alkoxy, deuterated C₁₋₆ alkoxy, NO₂, NH₂, or cyano. Y₁ represents halogen (such as fluorine, chlorine, or bromine), and Y₂, Y₃, Y₄, and Y₅ each independently represent N or CH, with the proviso that Y₂, Y₃, Y₄, and Y₅ are not all N at the same time. Rings W¹, W², t1, t2, (R^{c1})ₘ₁, (R^{c2})ₘ₂ are as defined in the compound of Formula (I) of the present disclosure. Rⁿ¹ represents Boc, H, or —NHRₕ, where Rₕ represents hydrogen or C₁₋₆ alkyl (such as methyl or ethyl). It should be understood that when ring W¹-Rⁿ¹ contains additional reactive groups (such as —NH— or —NH₂) that can participate in the reaction of Scheme M-11, the reactive groups can be protected by techniques and methods known to those skilled in the art, such as using protecting groups. The removal of protecting groups can be achieved through techniques and methods known to those skilled in the art. For example, the removal of the Boc protecting group can be achieved under acidic conditions such as hydrochloric acid or trifluoroacetic acid.

Depending upon the target compound, the reaction substrate, reaction conditions (including reaction dosage, temperature, time, etc.), and work up, etc. in Scheme M-11 can be appropriately modified and adjusted using techniques and methods well known to those skilled in the art to obtain the desired target compound. In Scheme M-11, the substitution reaction can be conventional techniques and methods known to those skilled in the art. For example, substitution reactions can be carried out under heating conditions in the presence of triethylamine/isopropanol, DIEA/ethanol, or NaI/DIEA/DMF.

### General Synthesis Method 12 for Intermediate Compounds

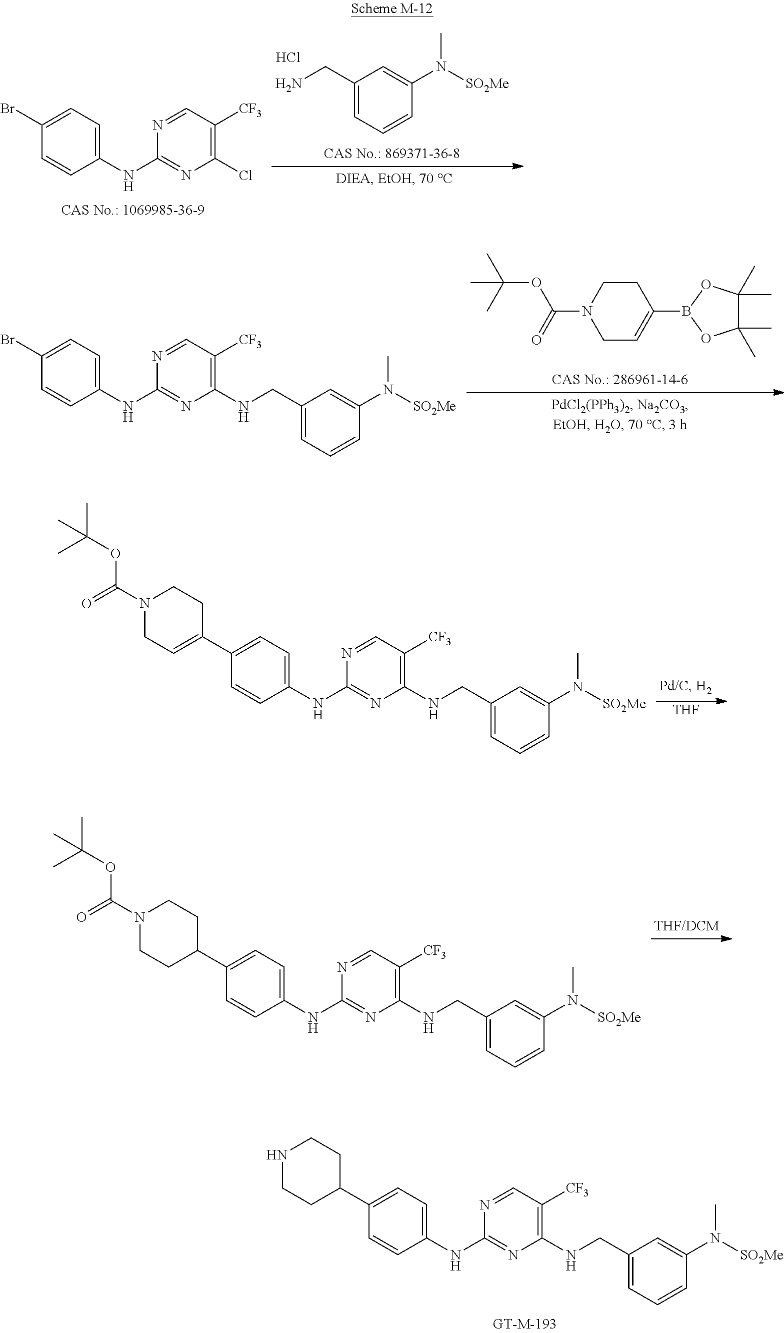

### General Synthesis Method 13 for Intermediate Compounds

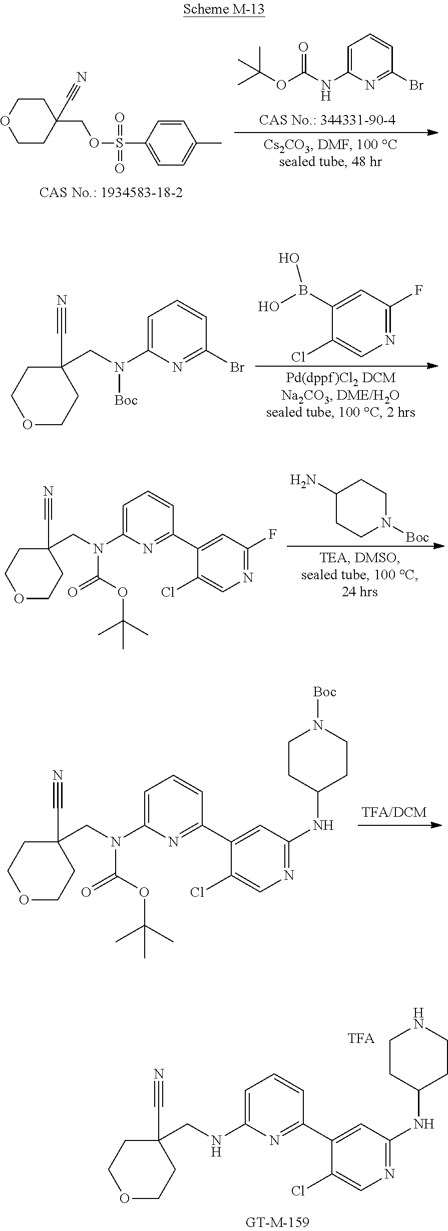

### General Synthesis Method 14 for Intermediate Compounds

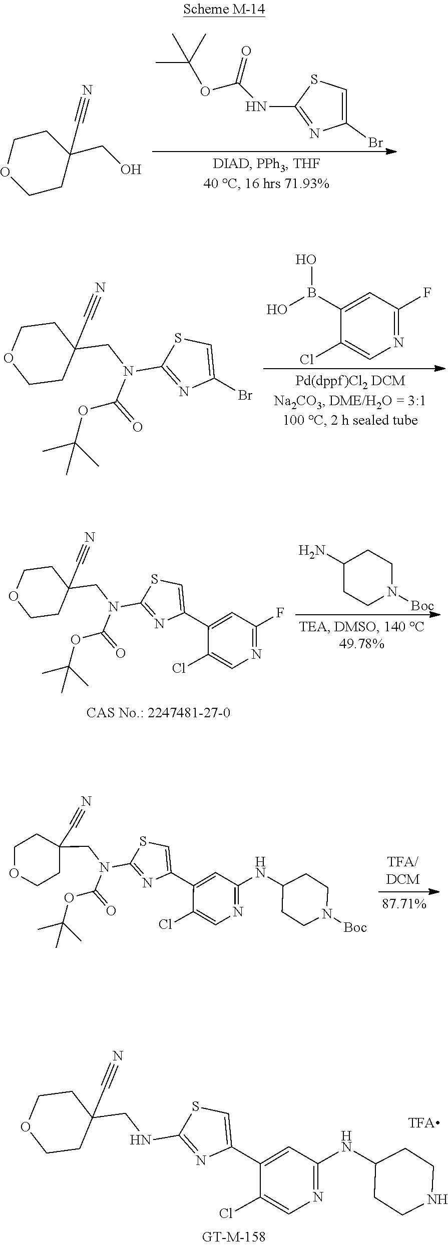

### General Synthesis Method 15 for Intermediate Compounds

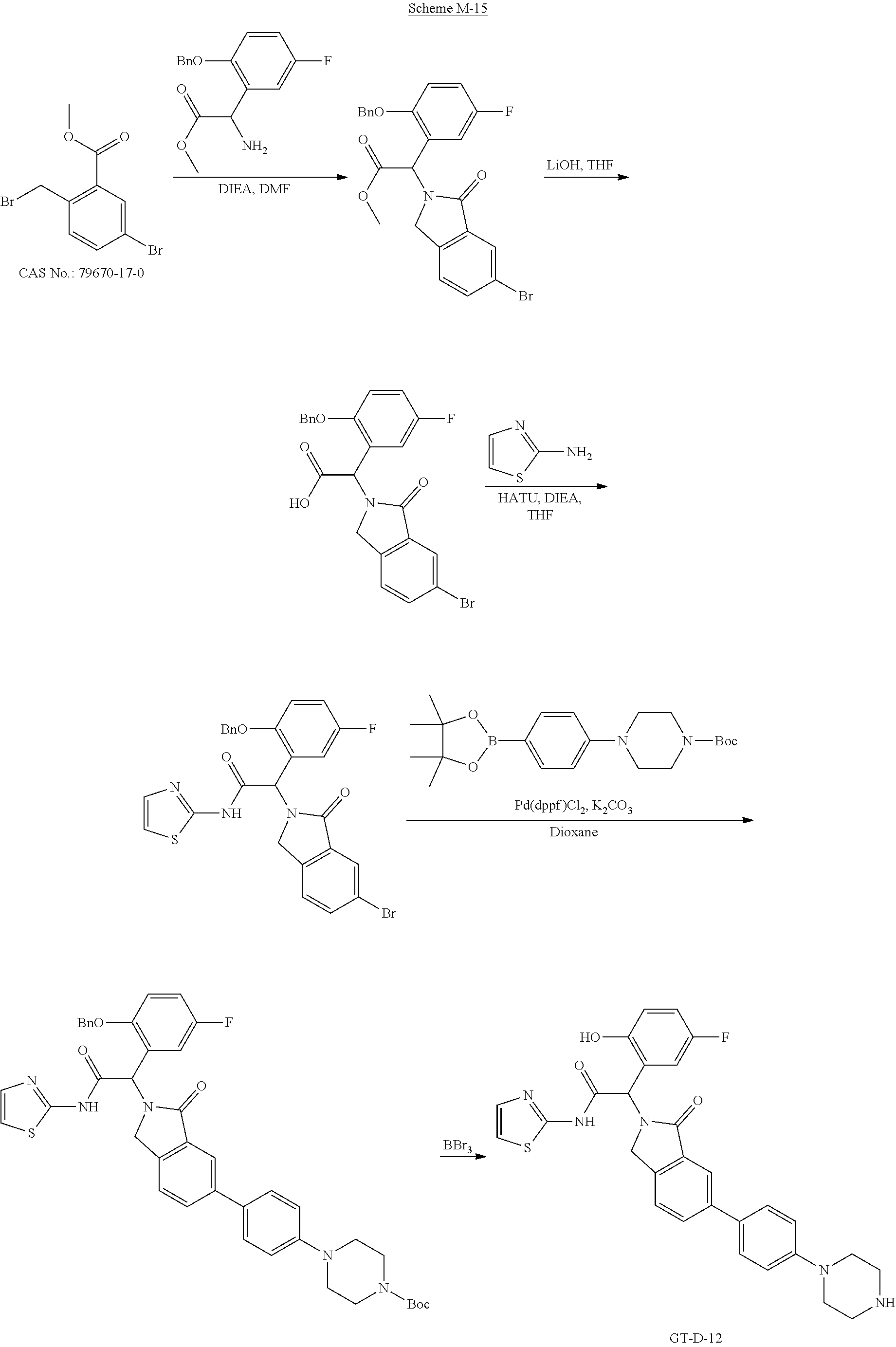

### General Synthesis Method 16 for Intermediate Compounds

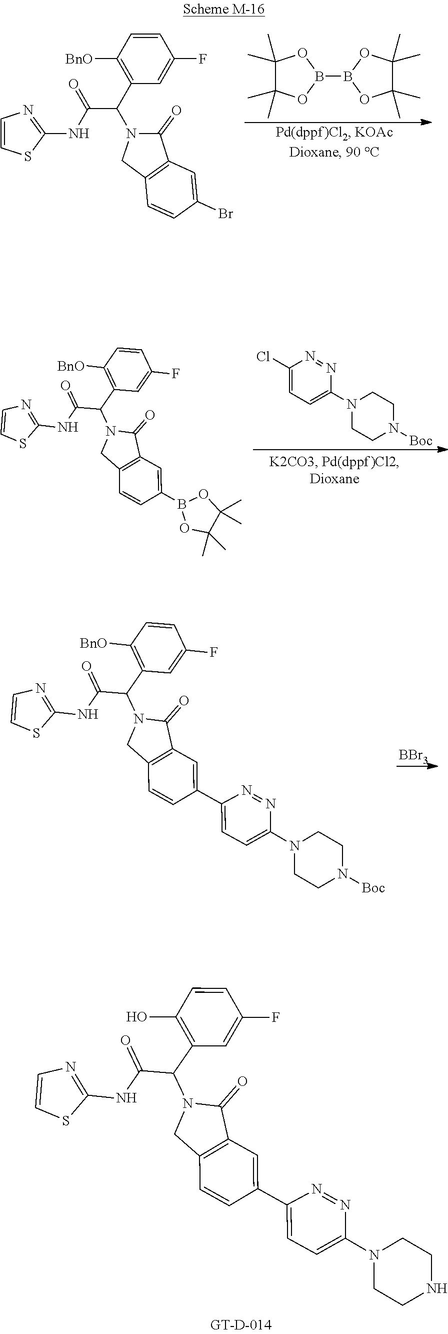

### General Synthesis Method 17 for Intermediate Compounds

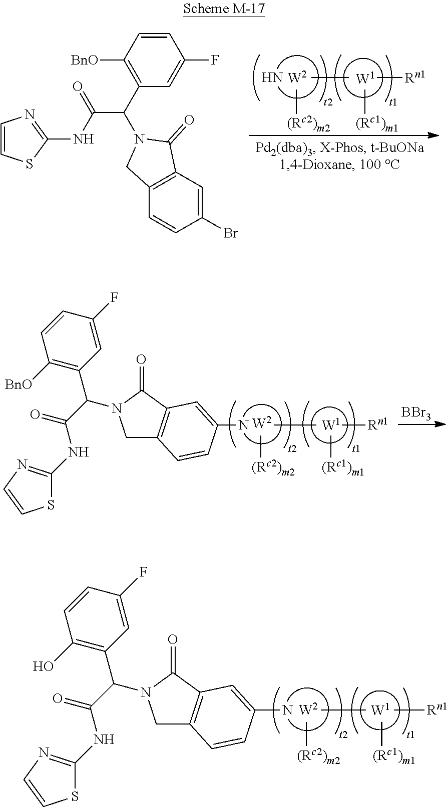

In Scheme M-17, the ring W¹, ring W², t1, t2, (R^{c1})ₘ₁, (R^{c2})ₘ₂ are as defined in the compound of Formula (I) of the present disclosure. Rⁿ¹ represents Boc, H, or —NHRₕ, where Rₕ represents hydrogen or C₁₋₆ alkyl (such as methyl or ethyl). It should be understood that when ring W¹-Rⁿ¹ contains additional reactive groups (such as —NH— or —NH₂) that can participate in the reaction of Scheme M-17, the reactive groups can be protected by techniques and methods known to those skilled in the art, such as using protecting groups. The removal of protecting groups can be achieved through techniques and methods known to those skilled in the art. For example, the removal of the Boc protecting group can be achieved under acidic conditions such as hydrochloric acid or trifluoroacetic acid.

Depending upon the target compound, the reaction substrate, reaction conditions (including reaction dosage, temperature, time, etc.), and work up, etc. in Scheme M-17 can be appropriately modified and adjusted using techniques and methods well known to those skilled in the art to obtain the desired target compound.

### General Synthesis Method 18 for Intermediate Compounds

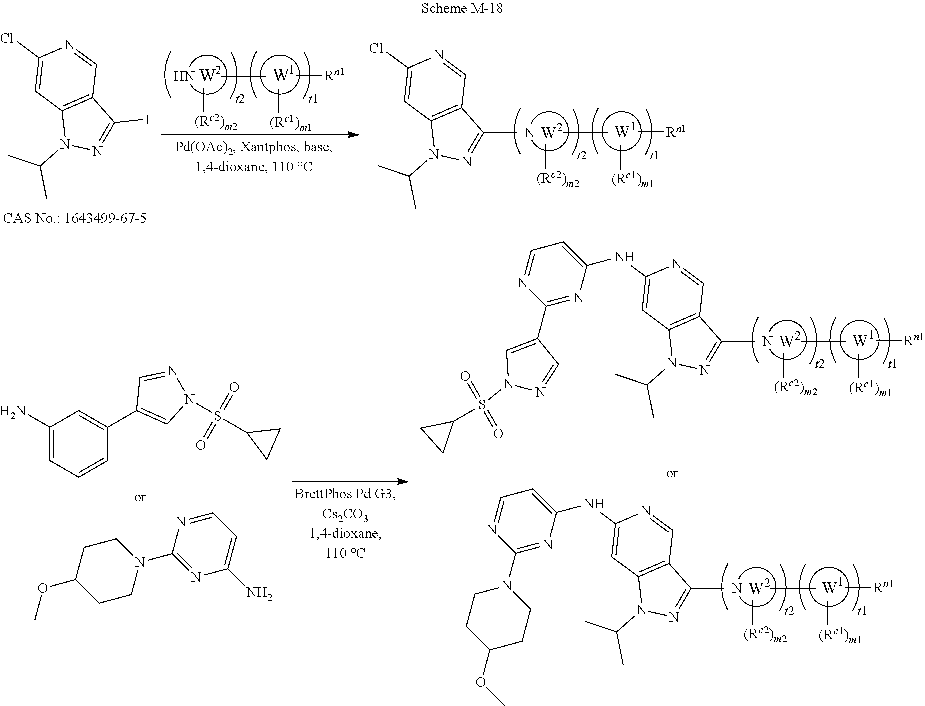

In Scheme M-18, the ring W¹, ring W², t1, t2, (R^{c1})ₘ₁, (R^{c2})ₘ₂ are as defined in the compound of Formula (I) of the present disclosure. Rⁿ¹ represents Boc, H, or —NHRₕ, where Rₕ represents hydrogen or C₁₋₆ alkyl (such as methyl or ethyl). It should be understood that when ring W¹-Rⁿ¹ contains additional reactive groups (such as —NH— or —NH₂) that can participate in the reaction of Scheme M-18, the reactive groups can be protected by techniques and methods known to those skilled in the art, such as using protecting groups. The removal of protecting groups can be achieved through techniques and methods known to those skilled in the art. For example, the removal of the Boc protecting group can be achieved under acidic conditions such as hydrochloric acid or trifluoroacetic acid.

### General Synthesis Method 19 for Intermediate Compounds

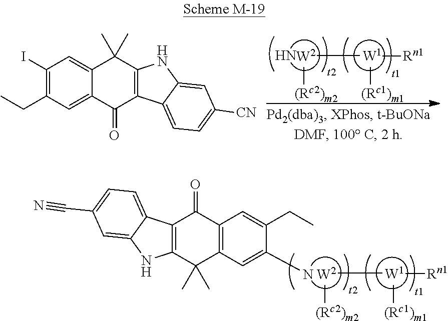

In Scheme M-19, the ring W¹, ring W², t1, t2, (R^{c1})ₘ₁, (R^{c2})ₘ₂ are as defined in the compound of Formula (I) of the present disclosure. Rⁿ¹ represents Boc, H, or —NHRₕ, where Rₕ represents hydrogen or C₁₋₆ alkyl (such as methyl or ethyl). It should be understood that when ring W¹-Rⁿ¹ contains additional reactive groups (such as —NH— or —NH₂) that can participate in the reaction of Scheme M-19, the reactive groups can be protected by techniques and methods known to those skilled in the art, such as using protecting groups. The removal of protecting groups can be achieved through techniques and methods known to those skilled in the art. For example, the removal of the Boc protecting group can be achieved under acidic conditions such as hydrochloric acid or trifluoroacetic acid.

### General Synthesis Method 20 For Intermediate Compounds

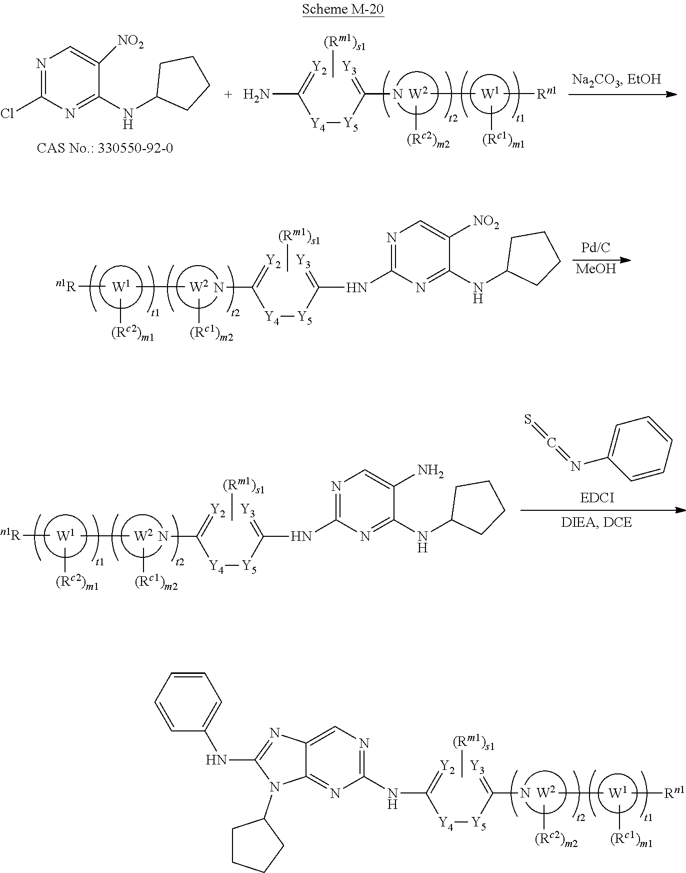

In Scheme M-20, (R^{m1})ₛ₁ of compounds indicates that the benzene ring is optionally substituted with s1 R^{m1} groups, with each R^{m1} being the same or different and independently representing halogen, hydroxy, C₁₋₆ alkyl (such as methyl or ethyl), halo C₁₋₆ alkyl, deuterated C₁₋₆ alkyl, C₁₋₆ alkoxy (such as methoxy or isopropoxy), halo C₁₋₆ alkoxy, deuterated C₁₋₆ alkoxy, NO₂, NH₂, or cyano. Y₁ represents halogen (such as fluorine, chlorine, or bromine), and Y₂, Y₃, Y₄, and Y₅ each independently represent N or CH, with the provision that Y₂, Y₃, Y₄, and Y₅ are not all N simultaneously. Ring W¹, ring W², t1, t2, (R^{c1})ₘ₁, (R^{c2})ₘ₂ are as defined in the compound of Formula (I) of the present disclosure. Rⁿ¹ represents Boc, H, or —NHRₕ, where Rₕ represents hydrogen or C₁₋₆ alkyl (such as methyl or ethyl). It should be understood that when ring W¹-Rⁿ¹ contains additional reactive groups (such as —NH— or —NH₂) that can participate in the reaction of Scheme M-20, the reactive groups can be protected by techniques and methods known to those skilled in the art, such as using protecting groups. The removal of protecting groups can be achieved through techniques and methods known to those skilled in the art. For example, the removal of the Boc protecting group can be achieved under acidic conditions such as hydrochloric acid or trifluoroacetic acid.

### General Synthesis Method 21 for Intermediate Compounds

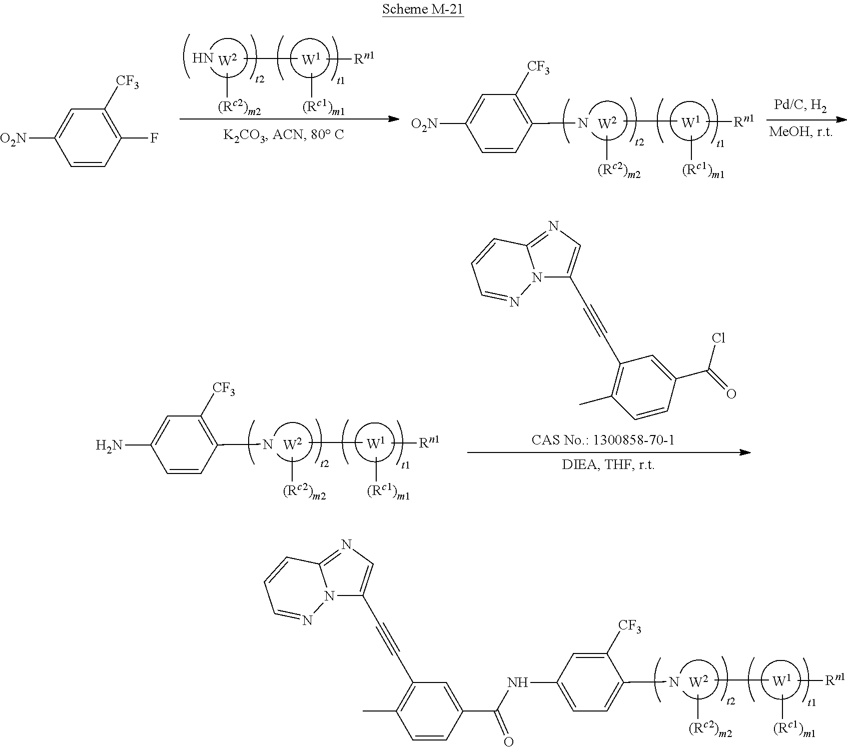

In Scheme 21, the rings W¹, W², t1, t2, (R^{c1})ₘ₁, (R^{c2})ₘ₂ are as defined in the compound of Formula (I) of the present disclosure. Rⁿ¹ represents Boc, H, or —NHRₕ, where Rₕ represents hydrogen or C₁₋₆ alkyl (such as methyl or ethyl). It should be understood that when ring W¹-Rⁿ¹ contains additional reactive groups (such as —NH— or —NH₂) that can participate in the reaction, the reactive groups can be protected by techniques and methods known to those skilled in the art, such as using protecting groups. The removal of protecting groups can be achieved through techniques and methods known to those skilled in the art. For example, the removal of the Boc protecting group can be achieved under acidic conditions (such as concentrated sulfuric acid, etc.).

### General Synthesis Method 22 for Intermediate Compounds

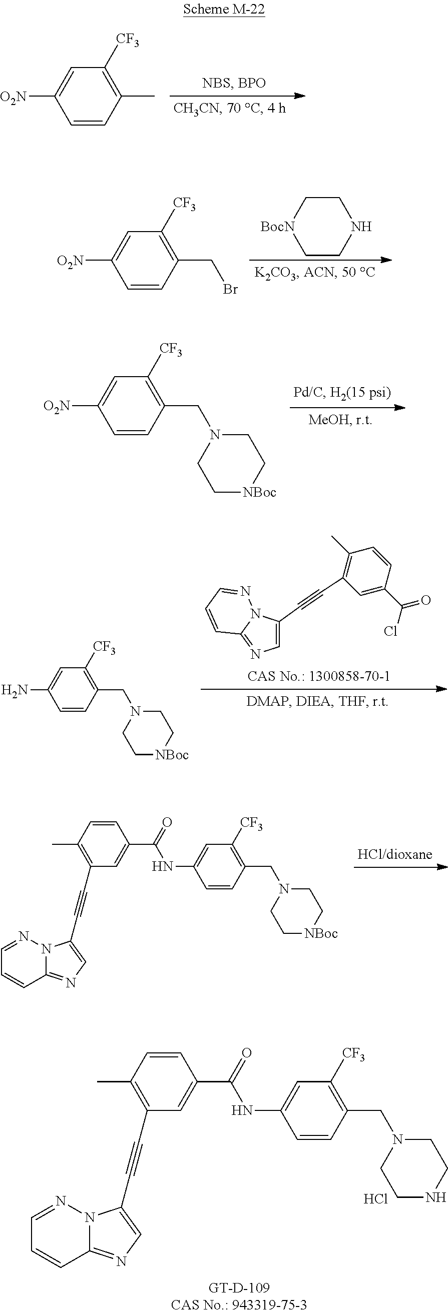

### General Synthesis Method 23 for Intermediate Compounds

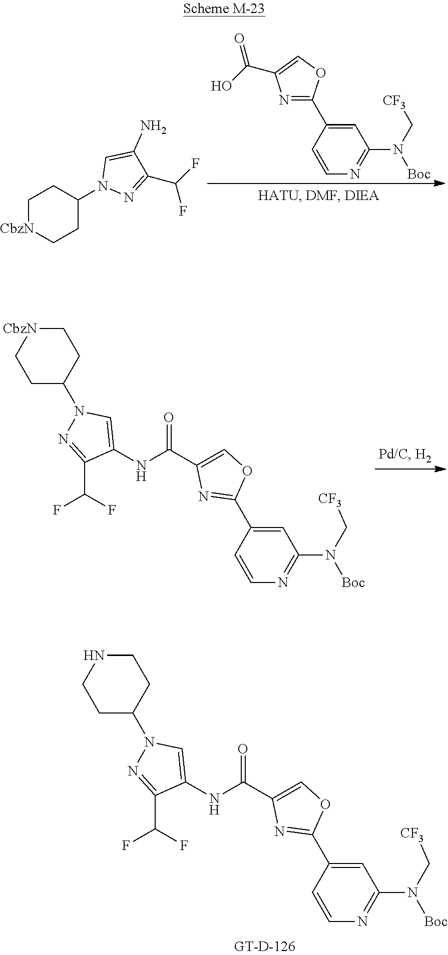 [SMILES: Nc1cn(C2CCN(C(=O)OCc3ccccc3)CC2)nc1C(F)F ; CC(C)(C)OC(=O)N(CC(F)(F)F)c1cc(-c2nc(C(=O)O)co2)ccn1 ; CC(C)(C)OC(=O)N(CC(F)(F)F)c1cc(-c2nc(C(=O)Nc3cn(C4CCN(C(=O)OCc5ccccc5)CC4)nc3C(F)F)co2)ccn1 ; CC(C)(C)OC(=O)N(CC(F)(F)F)c1cc(-c2nc(C(=O)Nc3cn(C4CCNCC4)nc3C(F)F)co2)ccn1]

### General Synthesis Method 24 for Intermediate Compounds

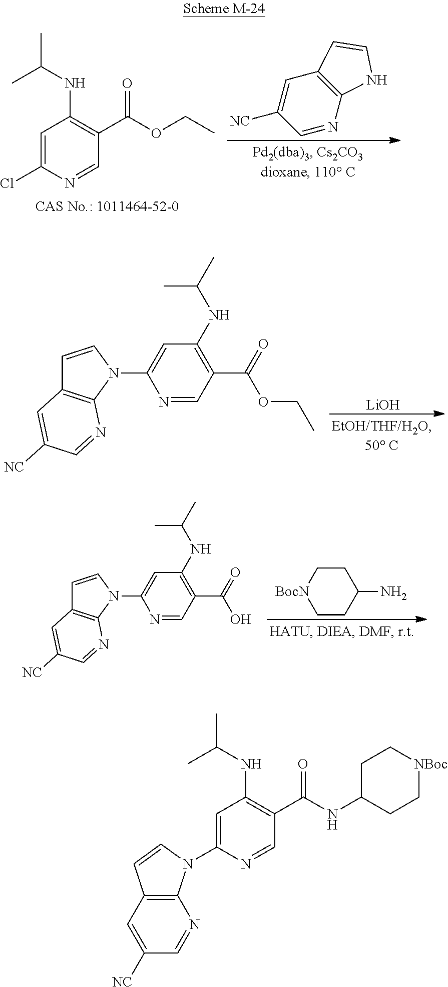

The removal of protecting groups can be achieved through techniques and methods known to those skilled in the art. For example, the removal of the Boc protecting group can be achieved under acidic conditions such as concentrated sulfuric acid.

### General Synthesis Method 25 for Intermediate Compounds

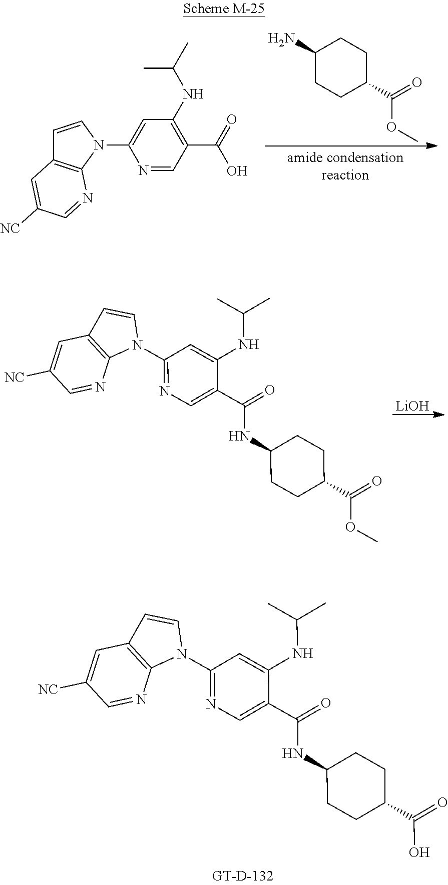 [SMILES: CC(C)Nc1cc(-n2ccc3cc(C#N)cnc32)ncc1C(=O)O ; COC(=O)[C@H]1CC[C@H](N)CC1 ; COC(=O)[C@H]1CC[C@H](NC(=O)c2cnc(-n3ccc4cc(C#N)cnc43)cc2NC(C)C)CC1 ; "LiOH" ; CC(C)Nc1cc(-n2ccc3cc(C#N)cnc32)ncc1C(=O)N[C@H]1CC[C@H](C(=O)O)CC1]

Depending upon the target compound, the reaction substrate, reaction conditions (including reaction dosage, temperature, time, etc.), and work up, etc. in Scheme M-25 can be appropriately modified and adjusted using techniques and methods well known to those skilled in the art to obtain the desired target compound. In Scheme M-25, the amide condensation reaction can be conventional techniques and methods known to those skilled in the art. For example, the amide condensation reaction can be carried out at room temperature in the presence of HATU/DIEA/DMF or HOAt/EDCI/TEA/DCM.

### General Synthesis Method 26 for Intermediate Compounds

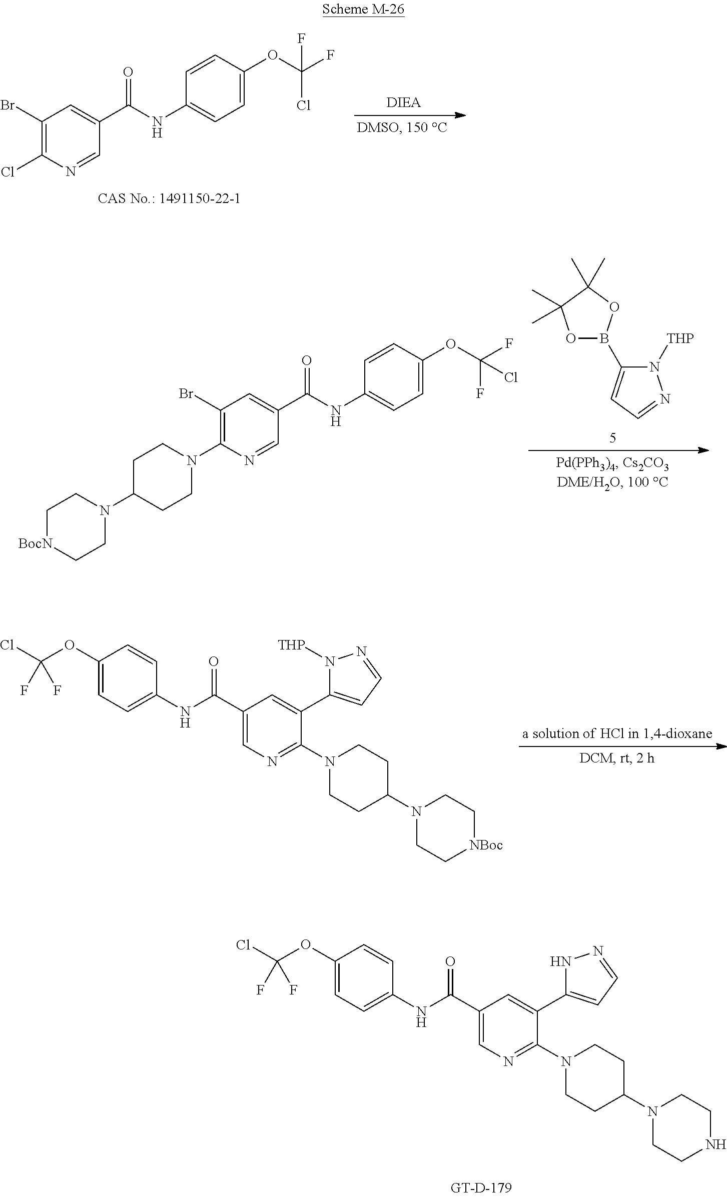

### General Synthesis Method 27 for Intermediate Compounds

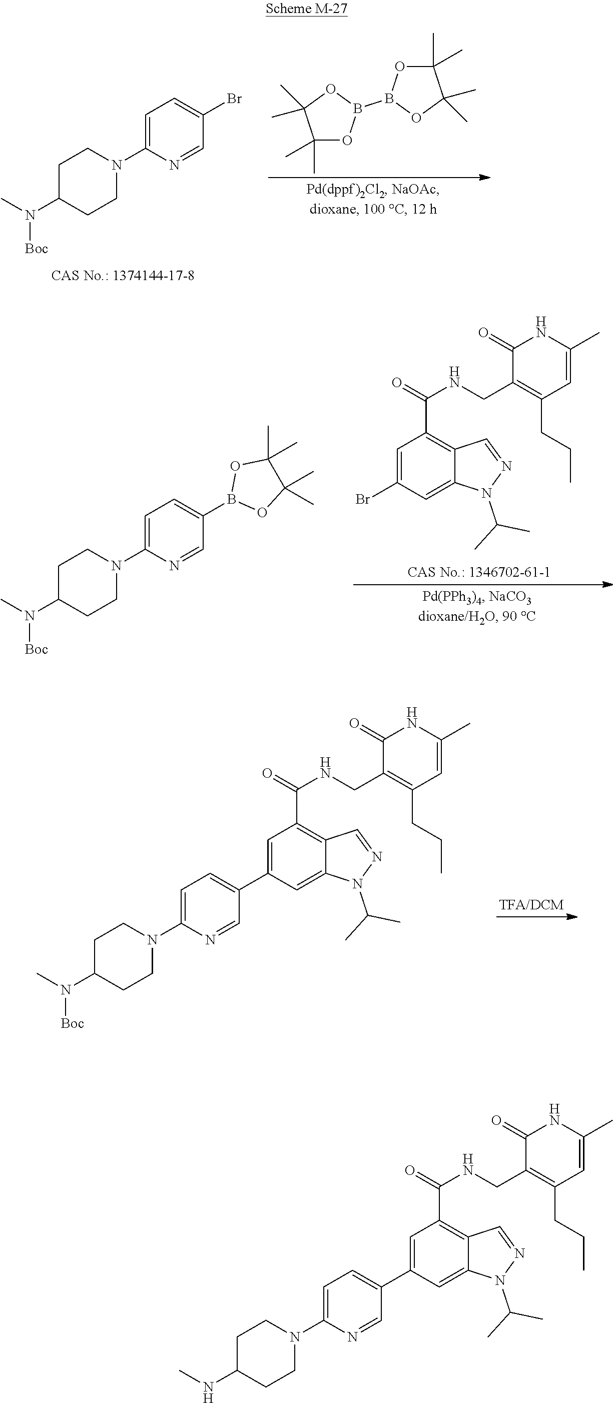

### General Synthesis Method 28 for Intermediate Compounds

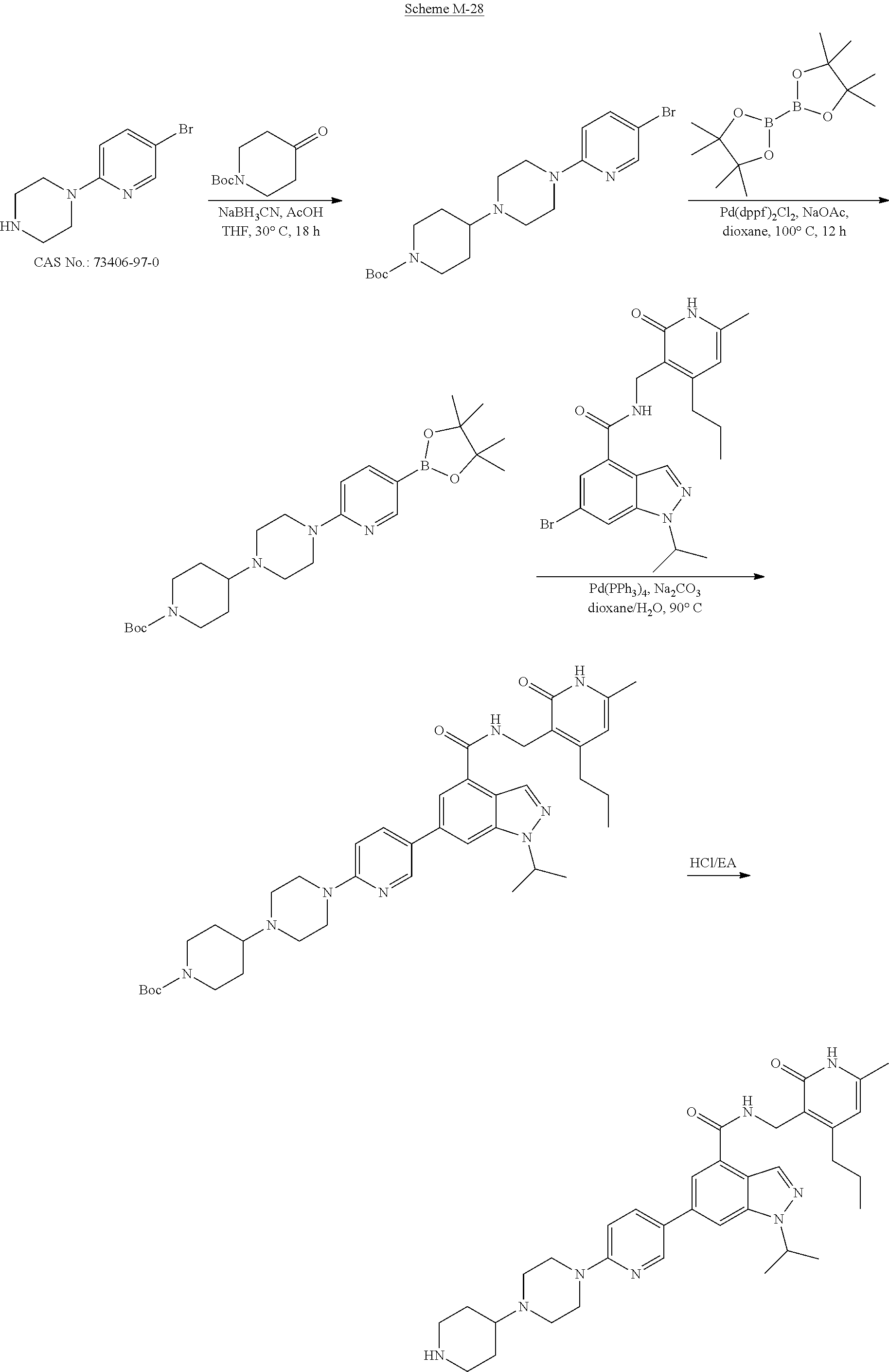

### General Synthesis Method 29 for Intermediate Compounds

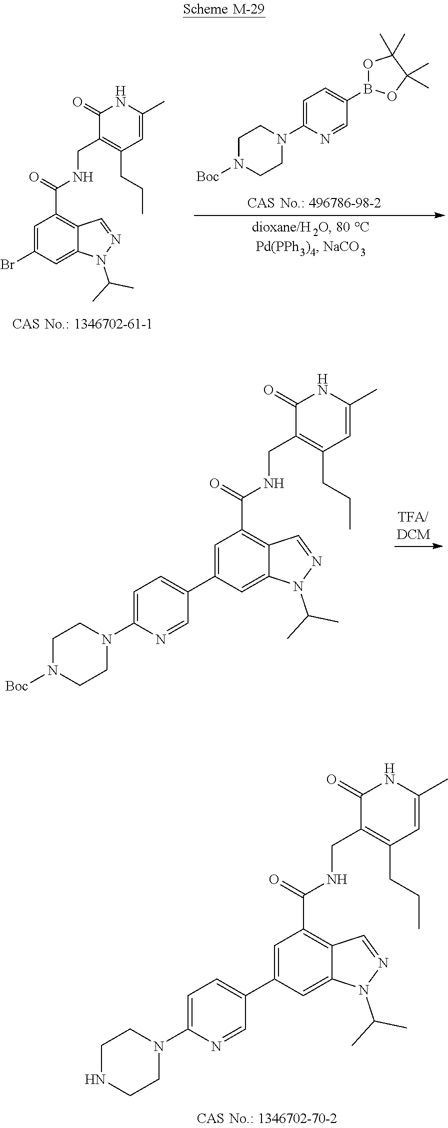

### General Synthesis Method 30 for Intermediate Compounds

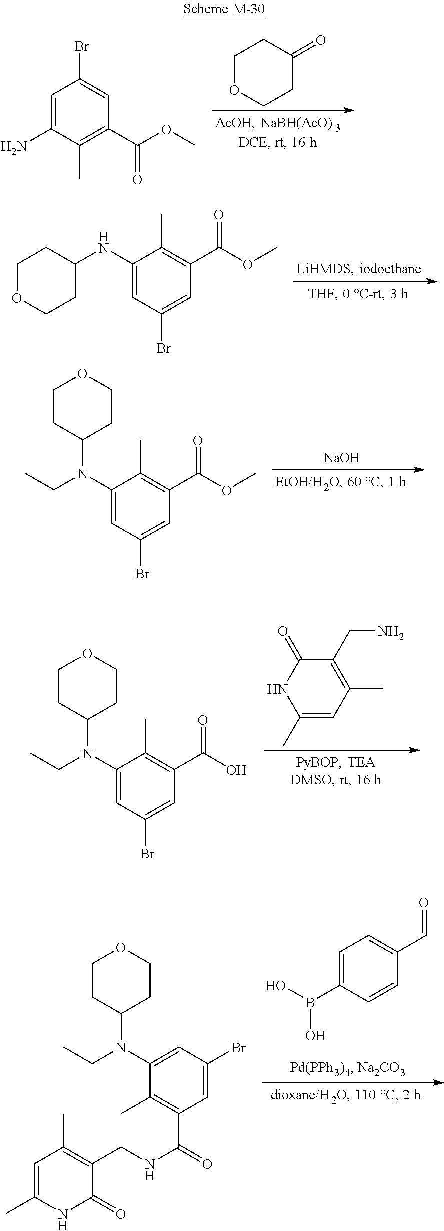

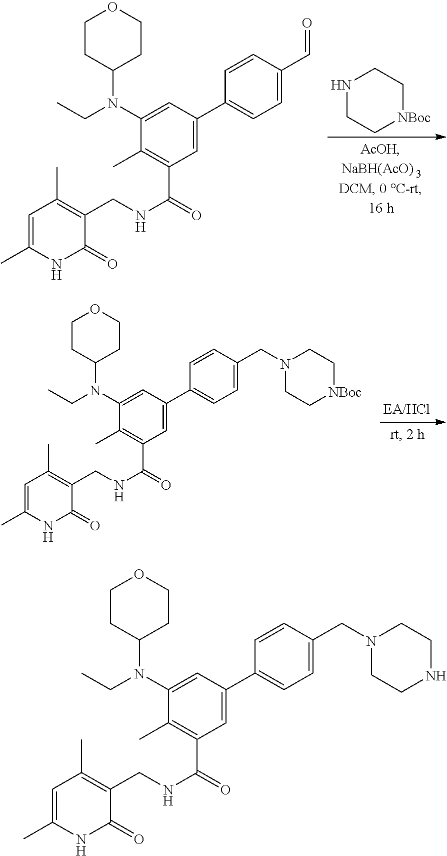

### General Synthesis Method 31 for Intermediate Compounds

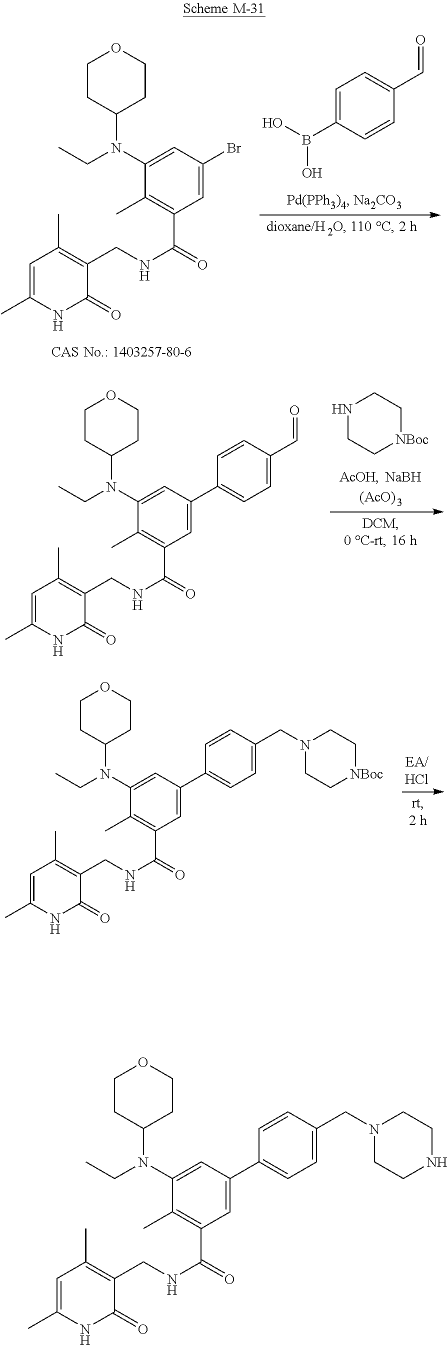

### General Synthesis Method 32 for Intermediate Compounds

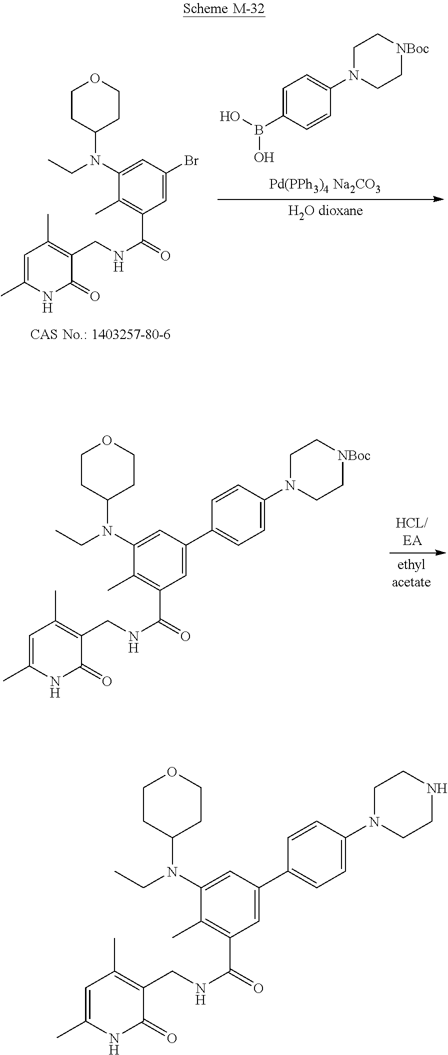

### General Synthesis Method 33 for Intermediate Compounds

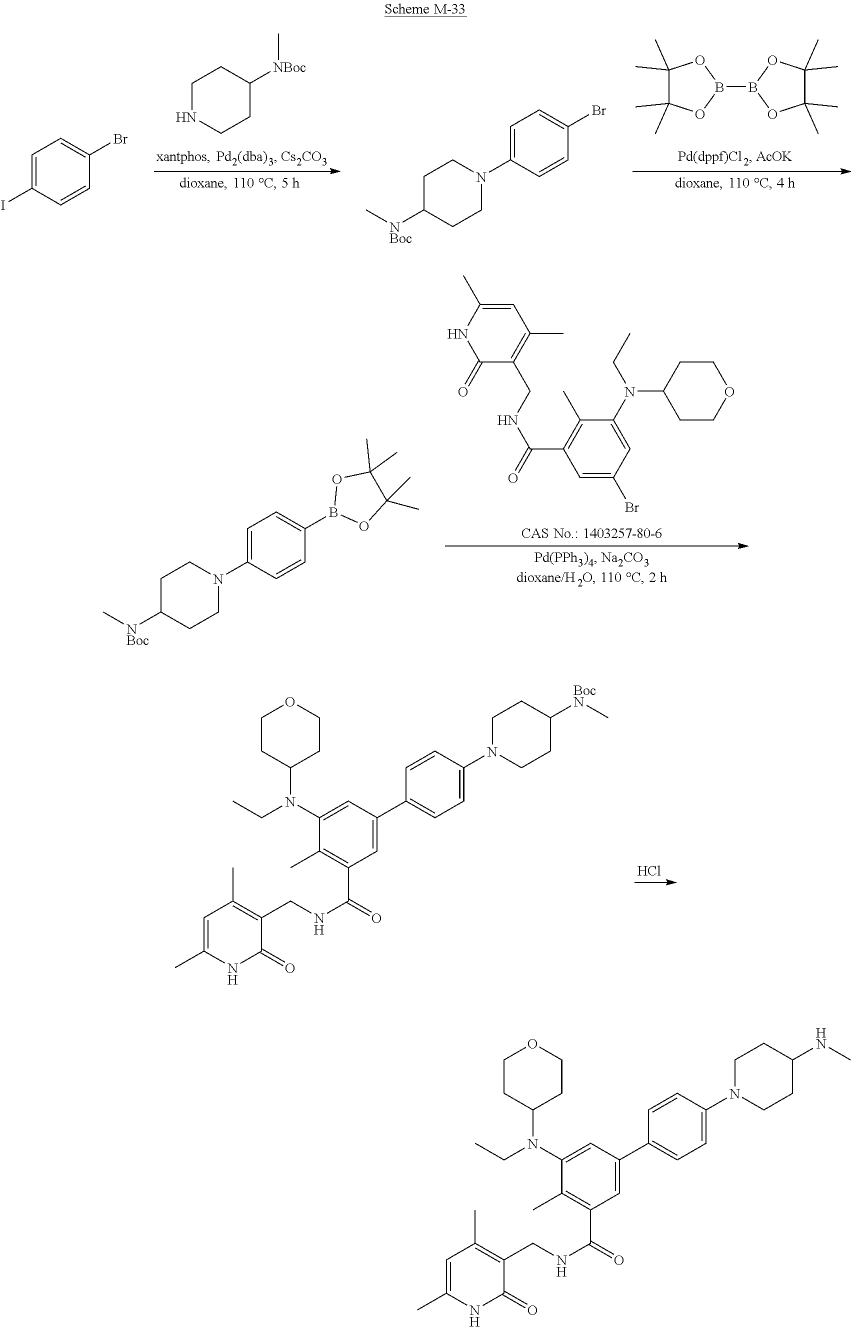

### General Synthesis Method 34 for Intermediate Compounds

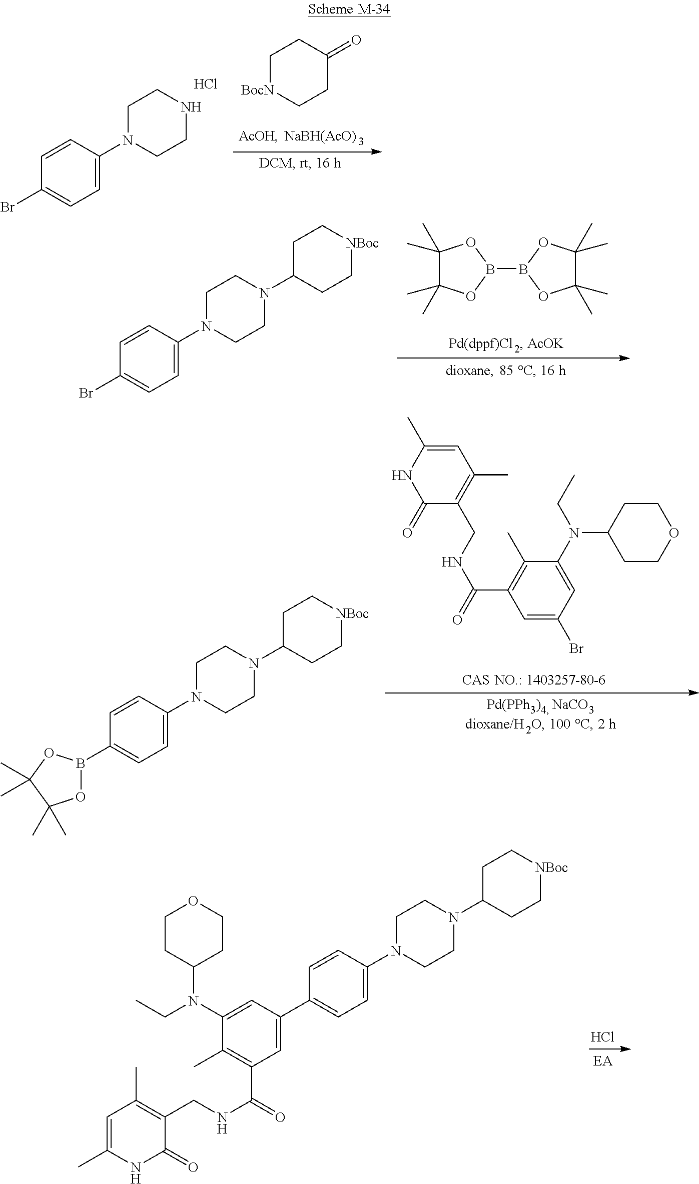

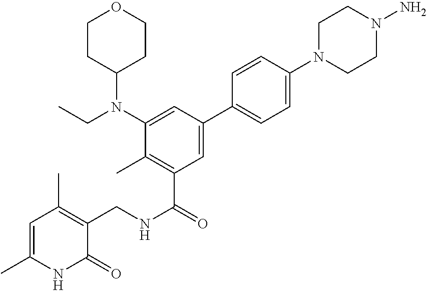

### General Synthesis Method 35 for Intermediate Compounds

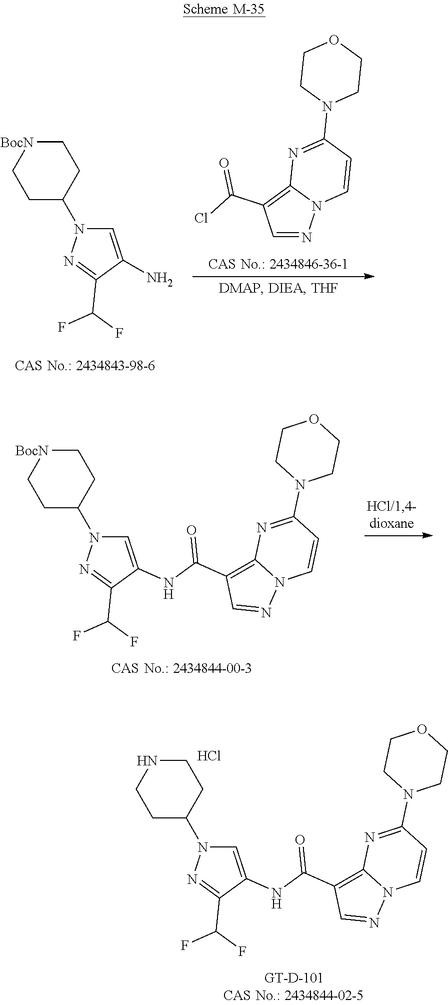

### General Synthesis Method 36 for Intermediate Compounds

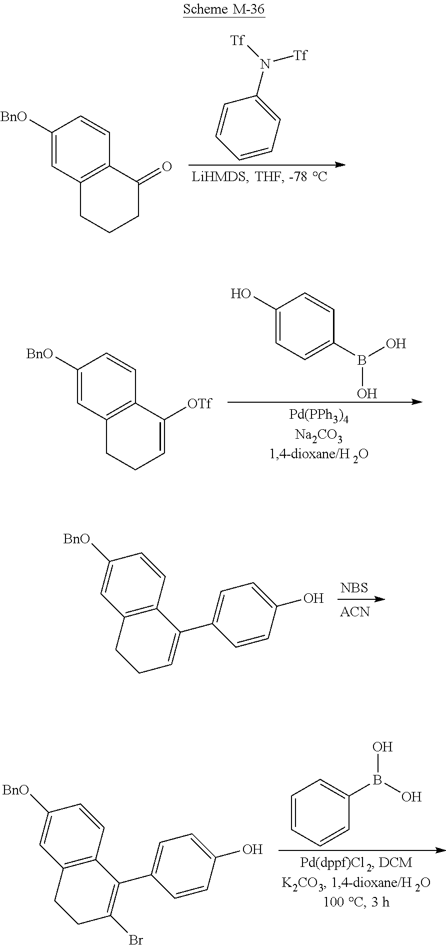

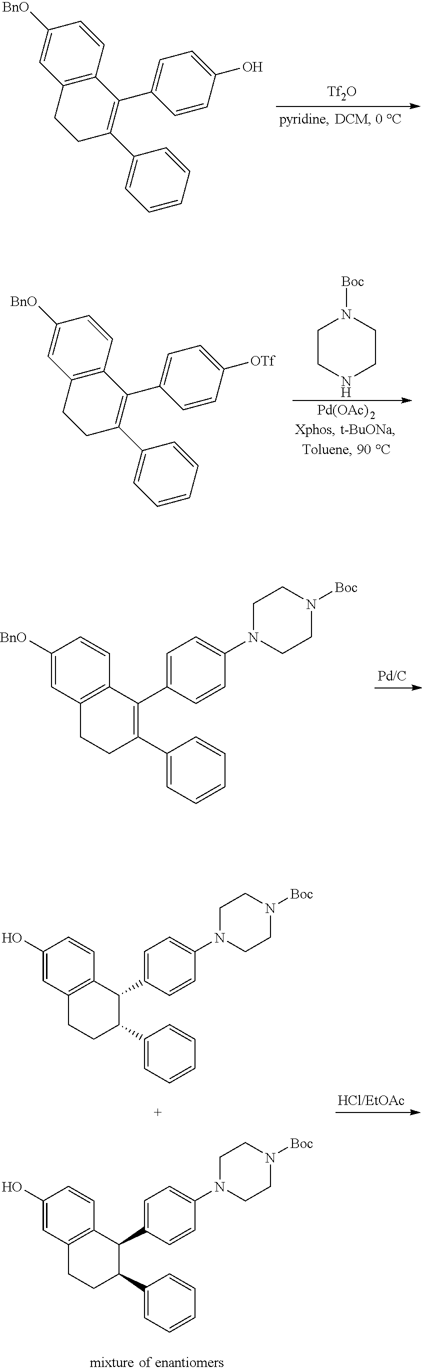

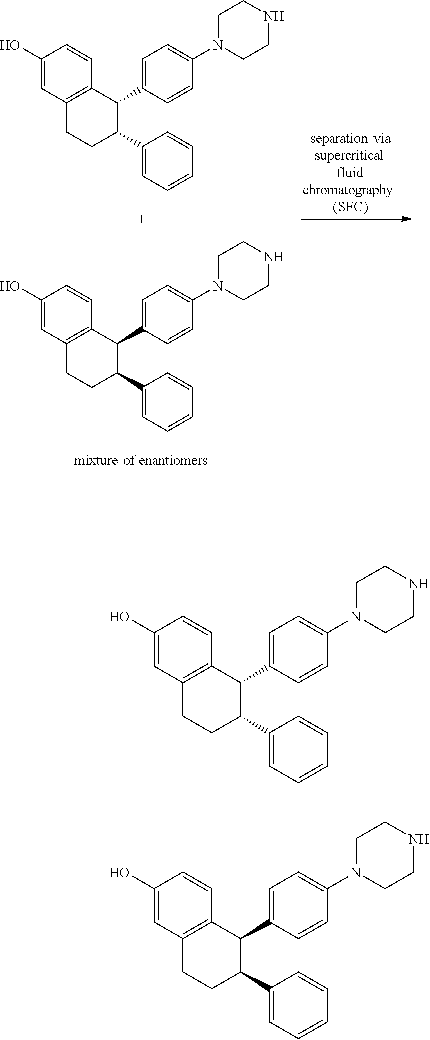

Pure enantiomeric products can be obtained through resolution/separation via supercritical fluid chromatography (SFC) and according to Scheme M-36. The conditions of supercritical fluid chromatography (SFC) can be appropriately modified and adjusted using techniques and methods well-known to those skilled in the art to obtain the desired target compound.

### General Synthesis Method 37 for Intermediate Compounds

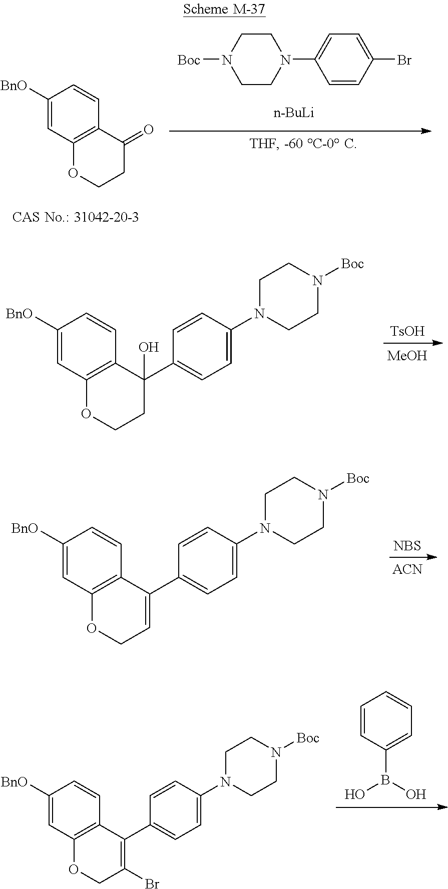

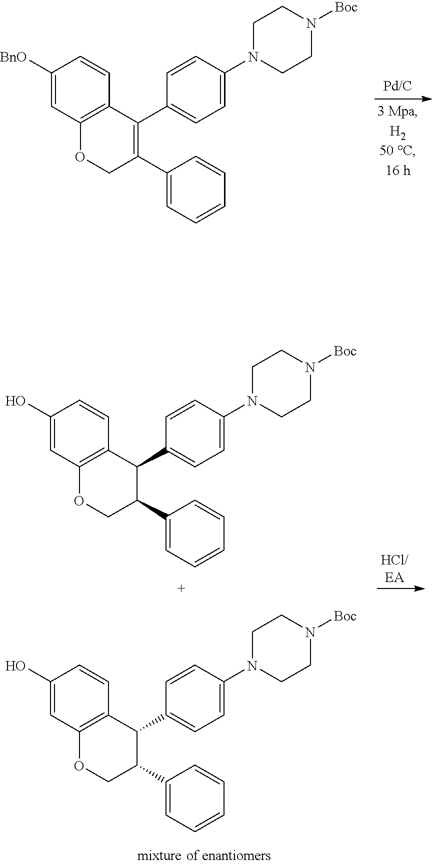

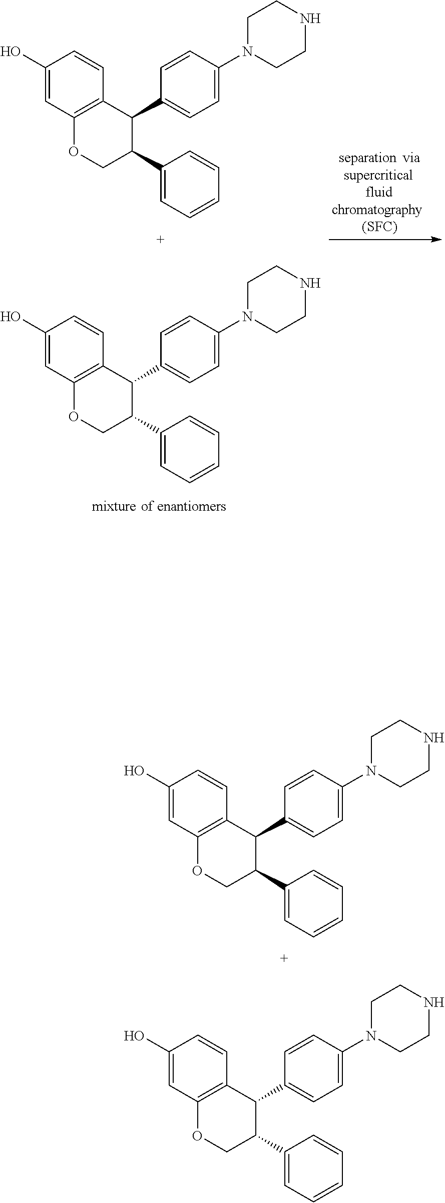

Pure enantiomeric products can be obtained through resolution/separation via supercritical fluid chromatography (SFC) and according to Scheme M-37. The conditions of supercritical fluid chromatography (SFC) can be appropriately modified and adjusted using techniques and methods well-known to those skilled in the art to obtain the desired target compound.

### General Synthesis Method 38 For Intermediate Compounds

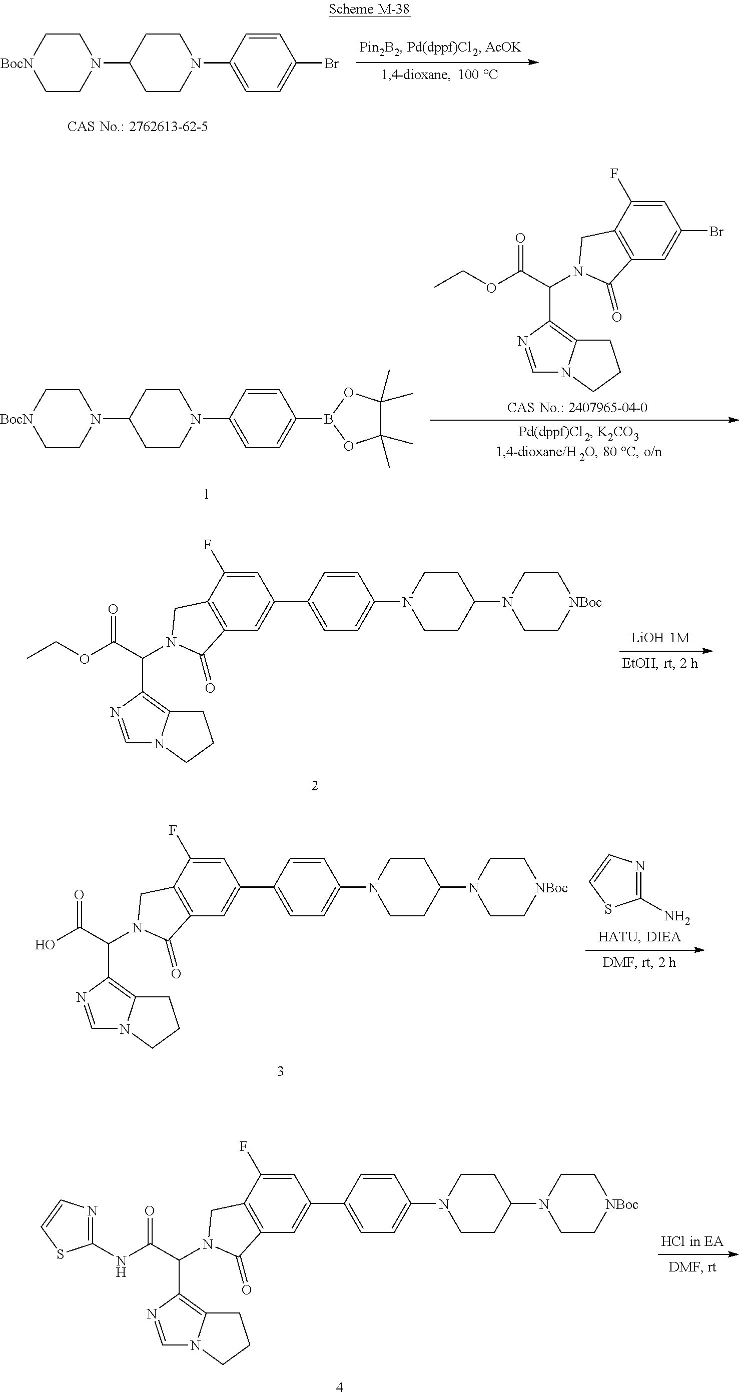

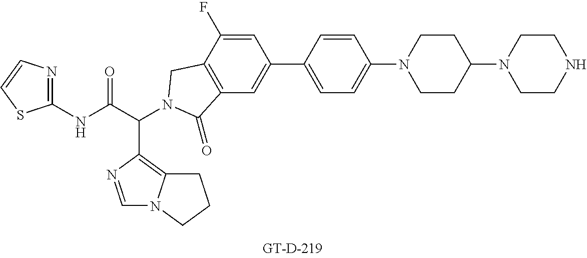

### General Synthesis Method 39 For Intermediate Compounds

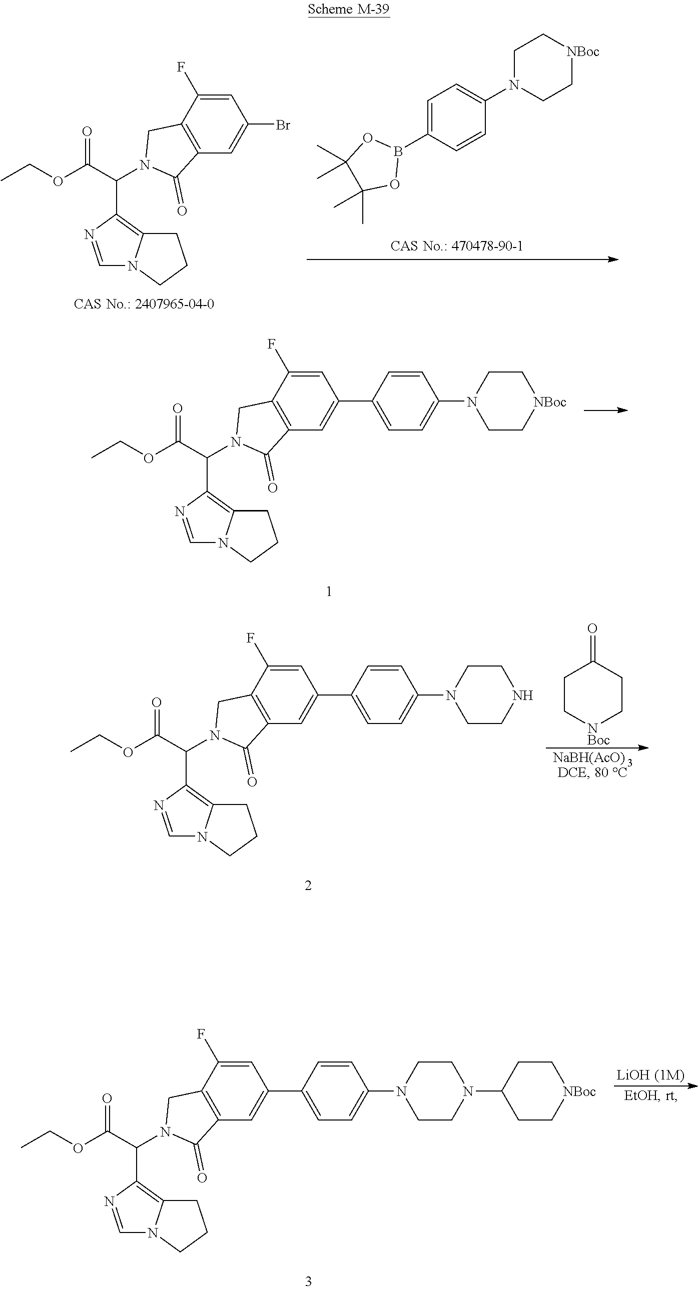

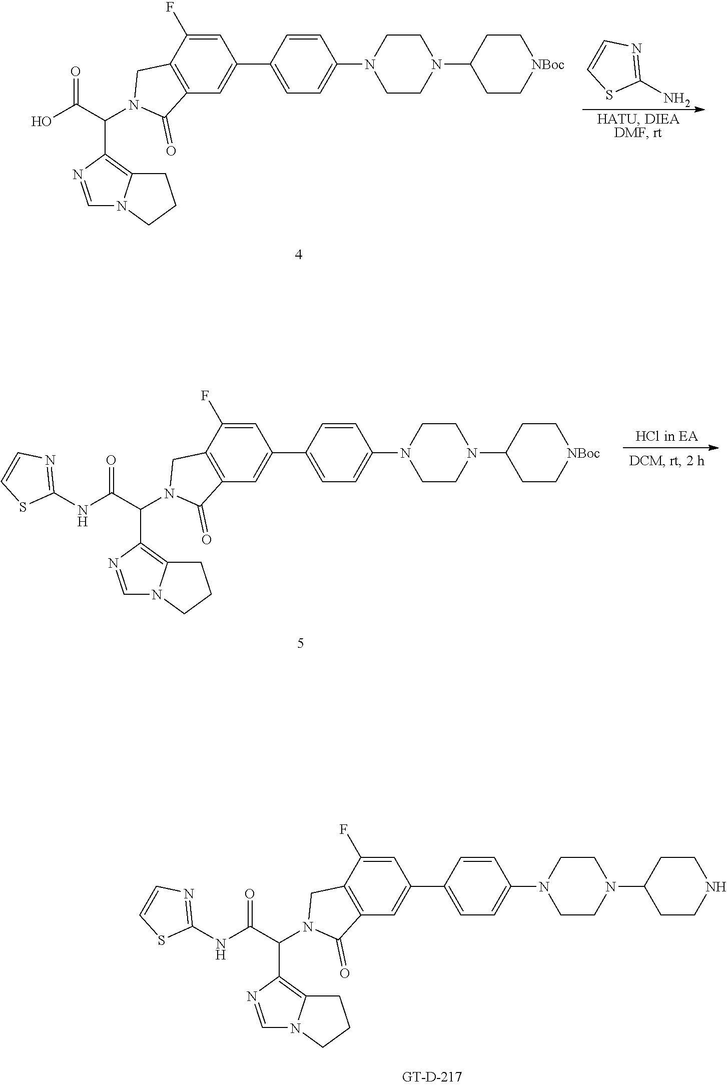

### General Synthesis Method 40 for Intermediate Compounds

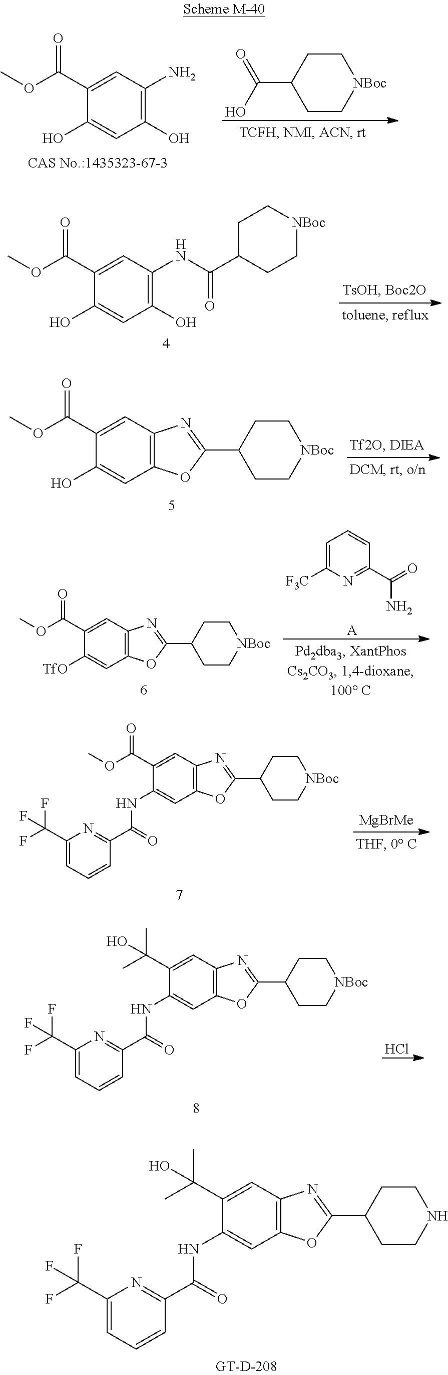

### General Synthesis Method 41 for Intermediate Compounds

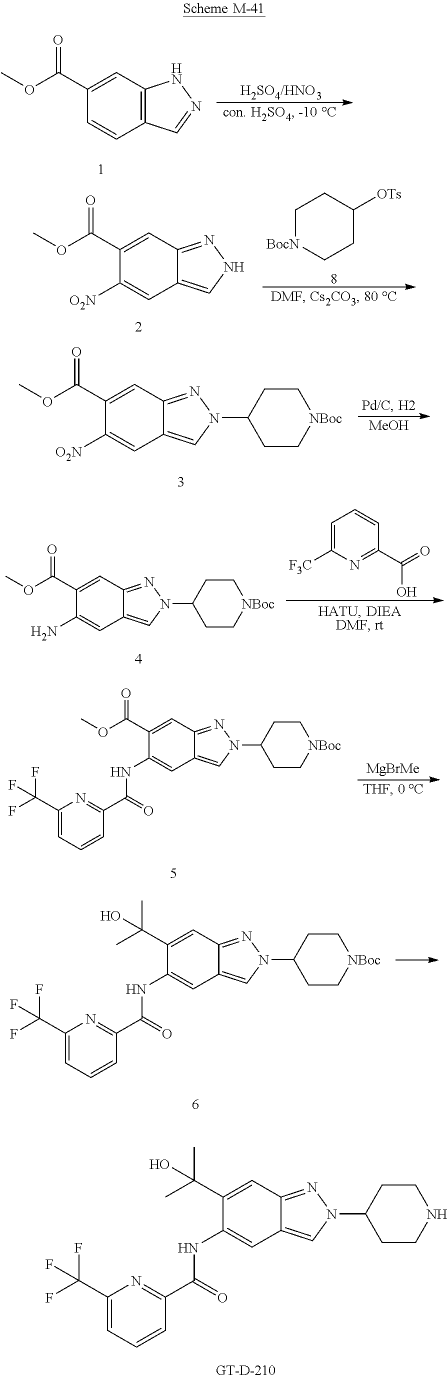

### General Synthesis Method 42 for Intermediate Compounds

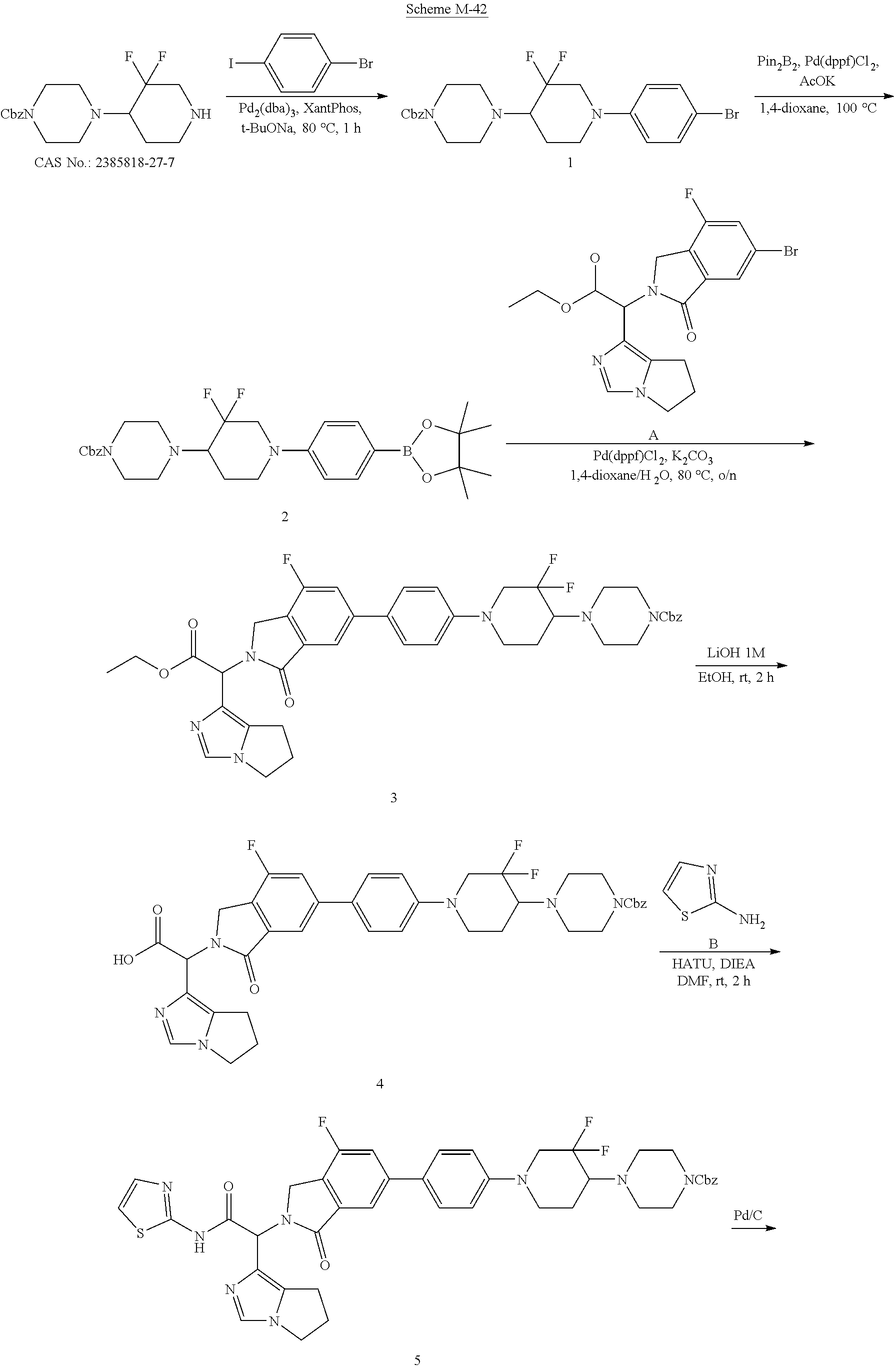

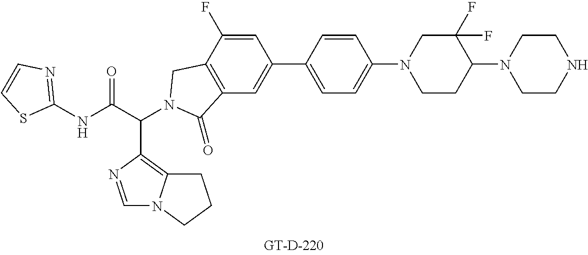

### General Synthesis Method 43 for Intermediate Compounds

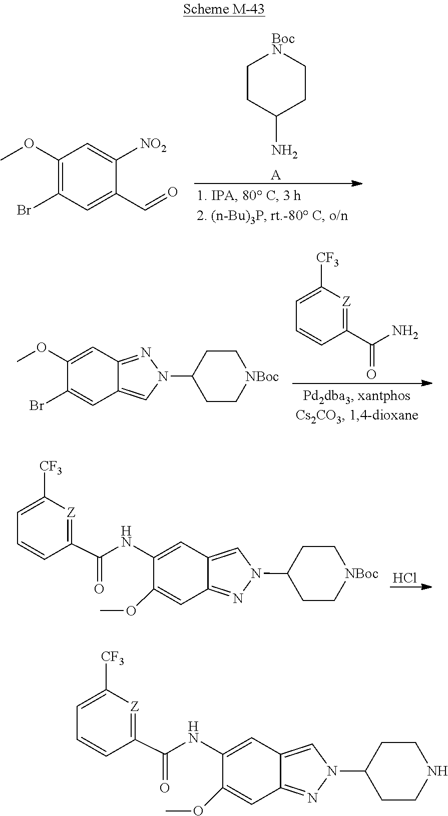

In Scheme M-43, Z represents CH or N.

### General Synthesis Method 44 for Intermediate Compounds

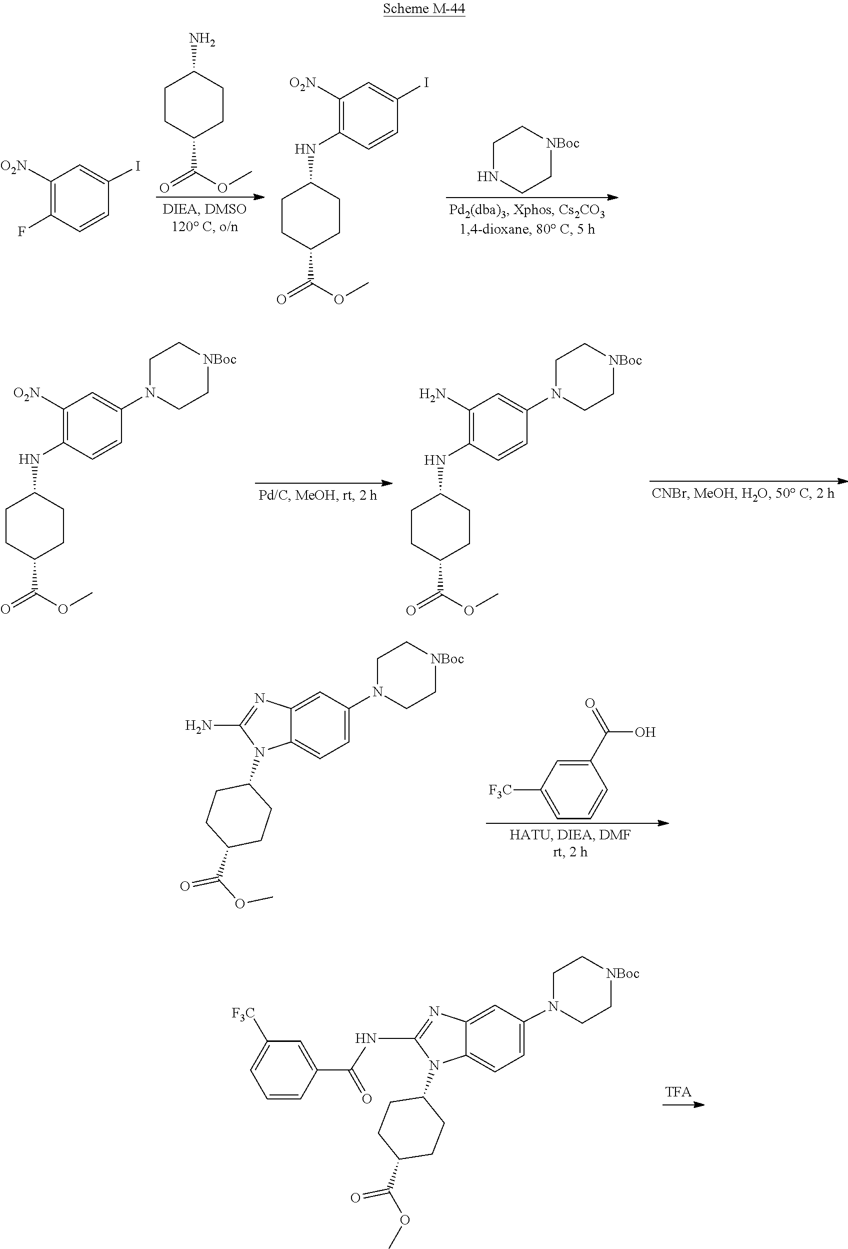

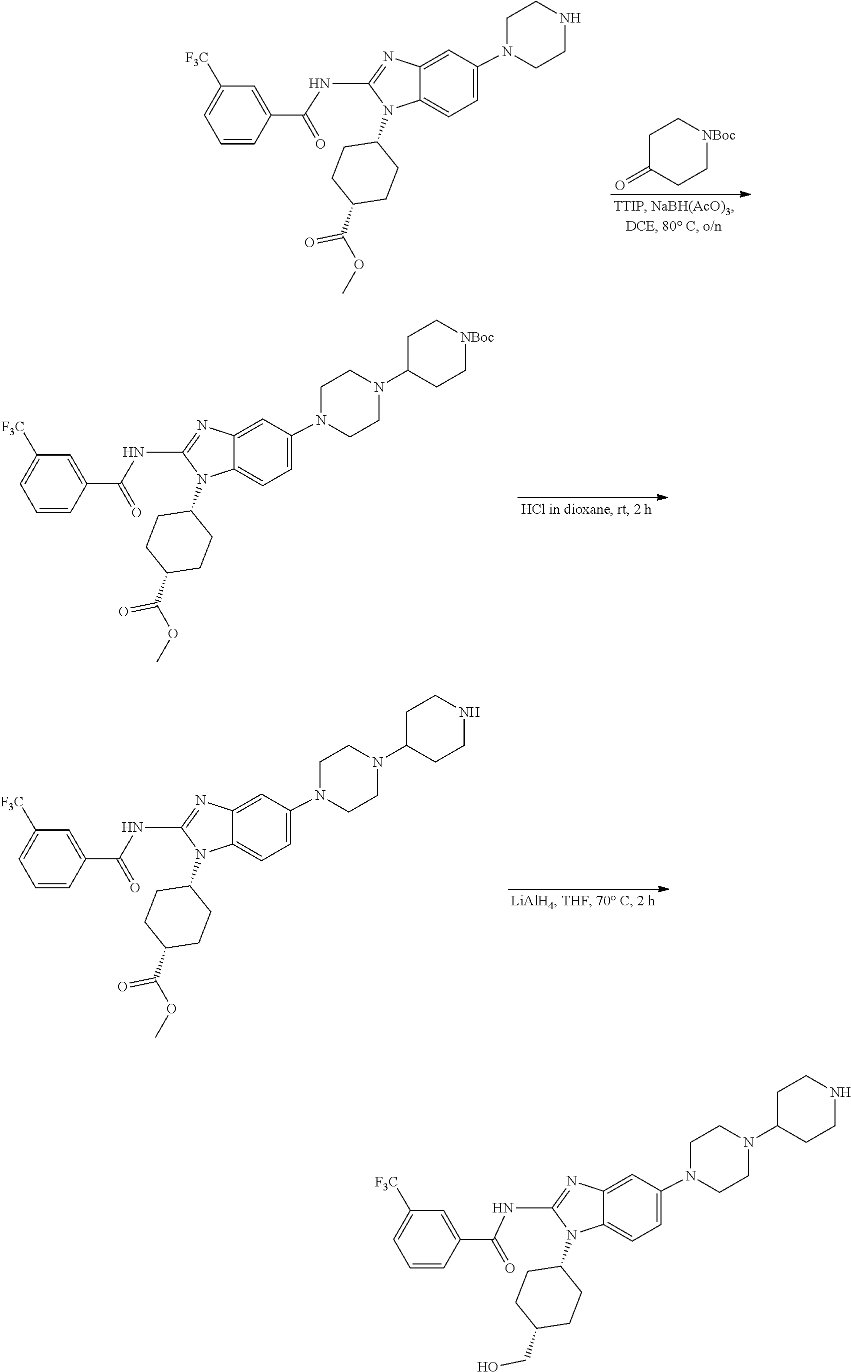 [SMILES: COC(=O)[C@H]1CC[C@@H](n2c(NC(=O)c3cccc(C(F)(F)F)c3)nc3cc(N4CCNCC4)ccc32)CC1 ; CC(C)(C)OC(=O)N1CCC(=O)CC1 ; COC(=O)[C@H]1CC[C@@H](n2c(NC(=O)c3cccc(C(F)(F)F)c3)nc3cc(N4CCN(C5CCN(C(=O)OC(C)(C)C)CC5)CC4)ccc32)CC1 ; COC(=O)[C@H]1CC[C@@H](n2c(NC(=O)c3cccc(C(F)(F)F)c3)nc3cc(N4CCN(C5CCNCC5)CC4)ccc32)CC1 ; O=C(Nc1nc2cc(N3CCN(C4CCNCC4)CC3)ccc2n1[C@H]1CC[C@@H](CO)CC1)c1cccc(C(F)(F)F)c1]

### General Synthesis Method 45 for Intermediate Compounds

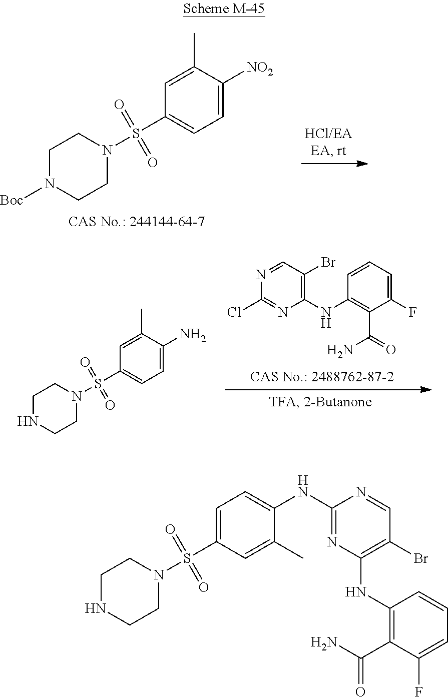 [SMILES: Cc1cc(S(=O)(=O)N2CCN(C(=O)OC(C)(C)C)CC2)ccc1[N+](=O)[O-] ; Cc1cc(S(=O)(=O)N2CCNCC2)ccc1N ; NC(=O)c1c(F)cccc1Nc1nc(Cl)ncc1Br ; Cc1cc(S(=O)(=O)N2CCNCC2)ccc1Nc1ncc(Br)c(Nc2cccc(F)c2C(N)=O)n1]

### General Synthesis Method 46 For Intermediate Compounds

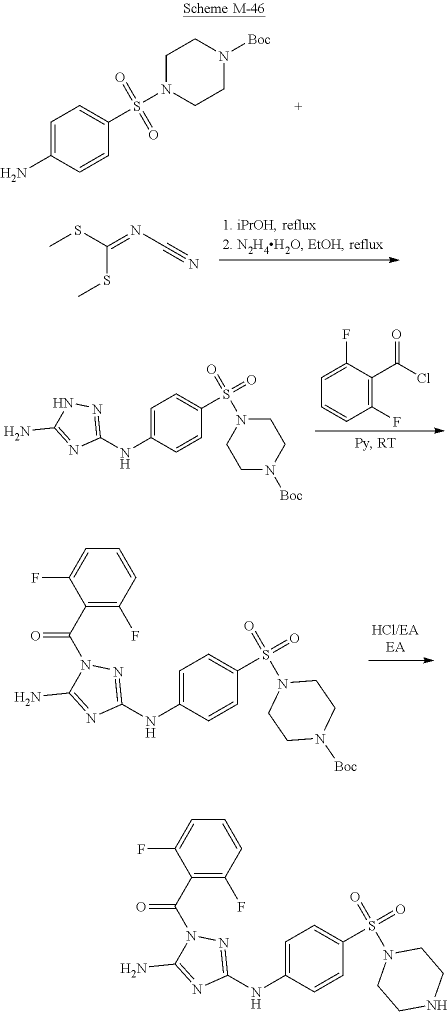

### General Synthesis Method 47 For Intermediate Compounds

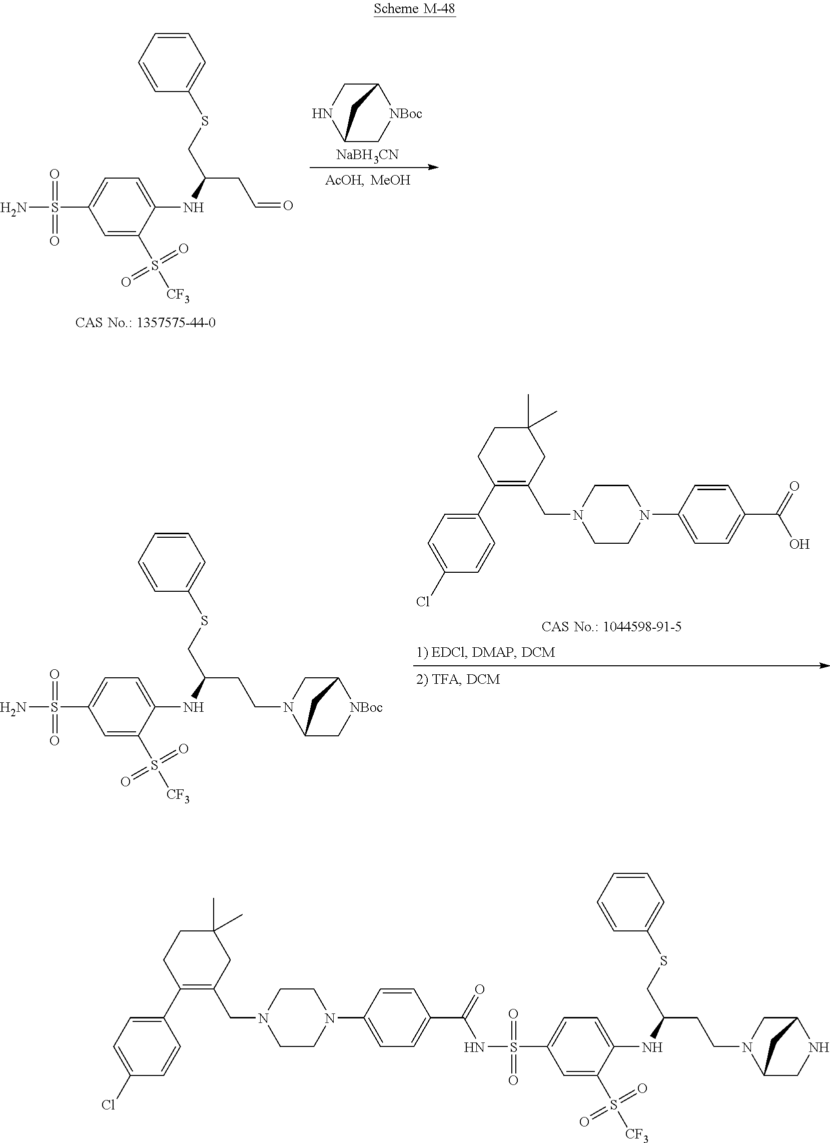

### General Synthesis Method 48 For Intermediate Compounds

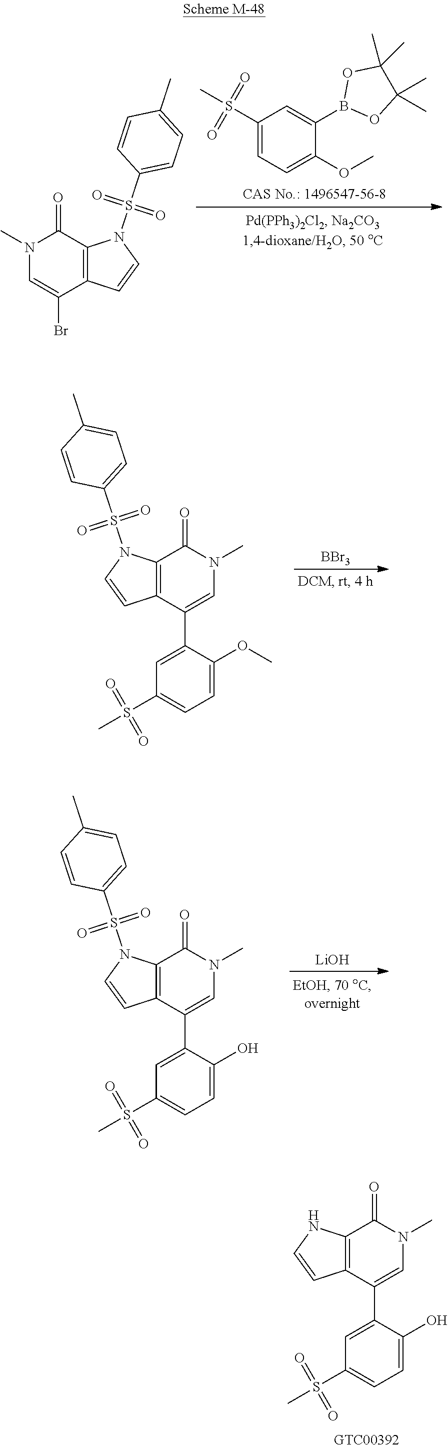

### General Synthesis Method 49 For Intermediate Compounds

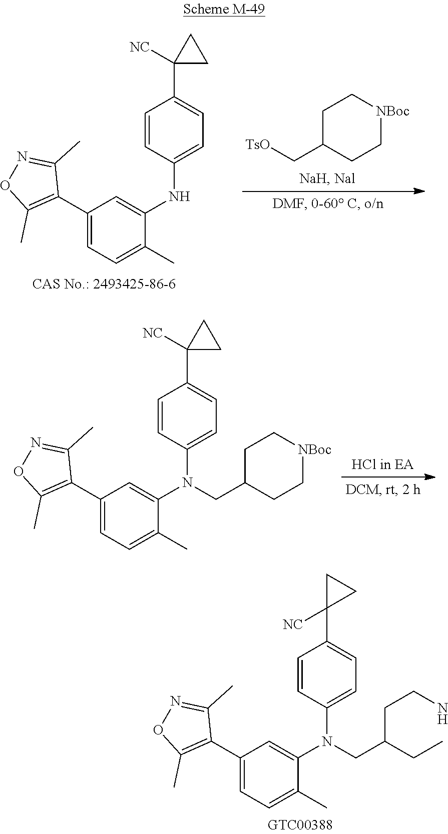 [SMILES: Cc1ccc(-c2c(C)noc2C)cc1Nc1ccc(C2(C#N)CC2)cc1 ; Cc1ccc(S(=O)(=O)OCC2CCN(C(=O)OC(C)(C)C)CC2)cc1 ; Cc1ccc(-c2c(C)noc2C)cc1N(CC1CCN(C(=O)OC(C)(C)C)CC1)c1ccc(C2(C#N)CC2)cc1 ; CCC(CCN)CN(c1ccc(C2(C#N)CC2)cc1)c1cc(-c2c(C)noc2C)ccc1C]

### General Synthesis Method 50 For Intermediate Compounds

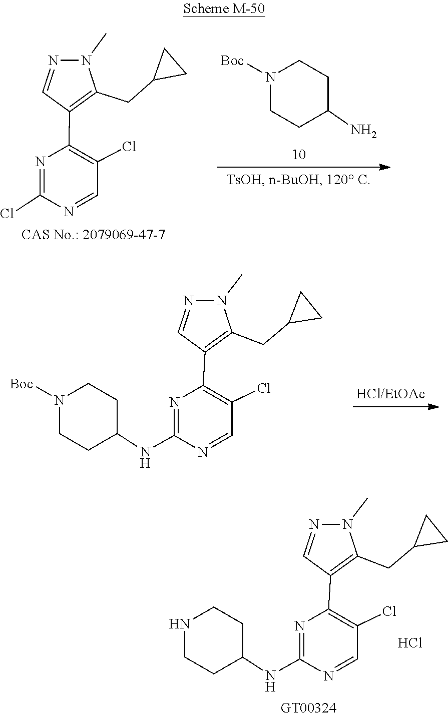 [SMILES: Cn1ncc(-c2nc(Cl)ncc2Cl)c1CC1CC1 ; CC(C)(C)OC(=O)N1CCC(N)CC1 ; Cn1ncc(-c2nc(NC3CCN(C(=O)OC(C)(C)C)CC3)ncc2Cl)c1CC1CC1 ; Cn1ncc(-c2nc(NC3CCNCC3)ncc2Cl)c1CC1CC1 ; Cl]

### General Synthesis Method 51 For Intermediate Compounds

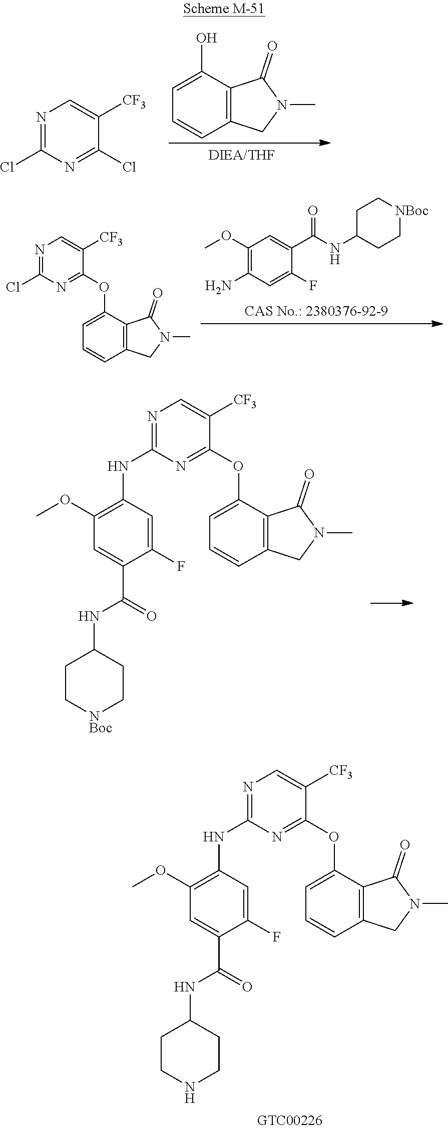

### General Synthesis Method 52 For Intermediate Compounds

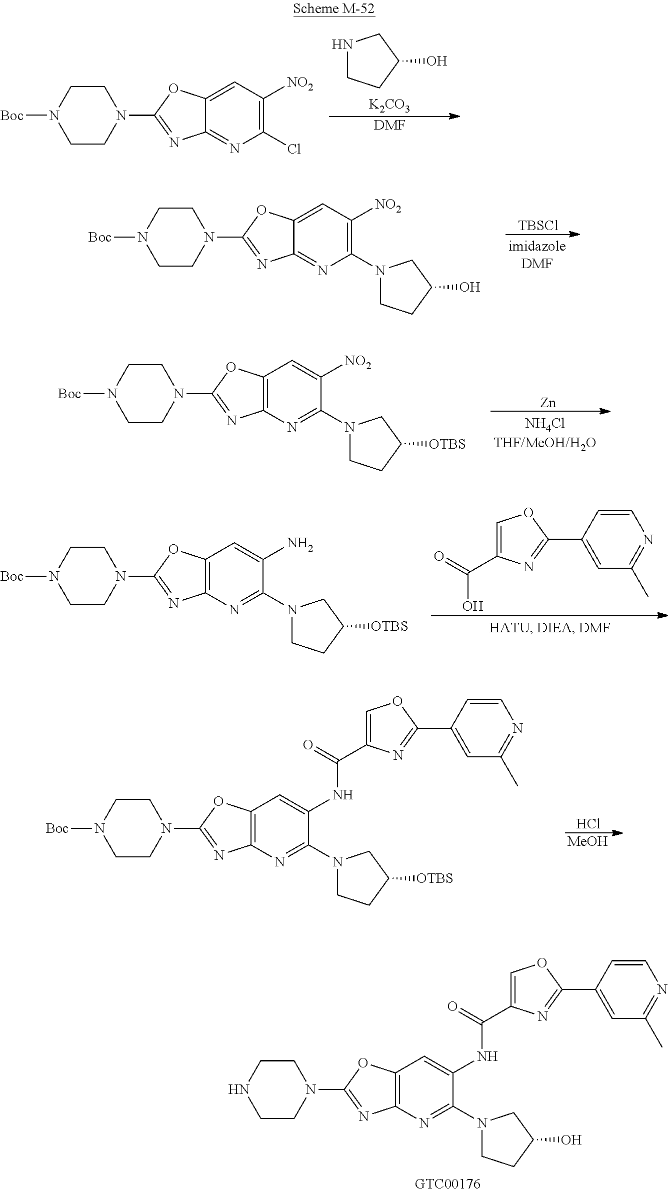

### General Synthesis Method 53 For Intermediate Compounds

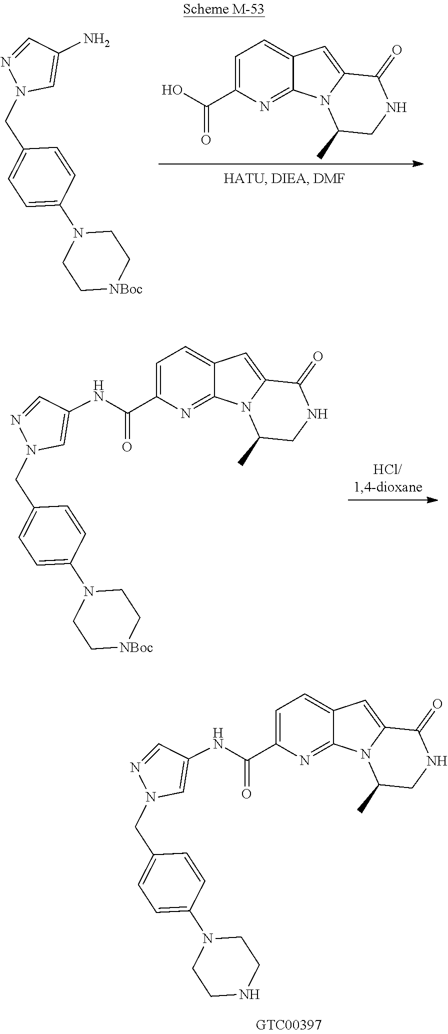 [SMILES: CC(C)(C)OC(=O)N1CCN(c2ccc(Cn3cc(N)cn3)cc2)CC1 ; C[C@@H]1CNC(=O)c2cc3ccc(C(=O)O)nc3n21 ; C[C@@H]1CNC(=O)c2cc3ccc(C(=O)Nc4cnn(Cc5ccc(N6CCN(C(=O)OC(C)(C)C)CC6)cc5)c4)nc3n21 ; C[C@@H]1CNC(=O)c2cc3ccc(C(=O)Nc4cnn(Cc5ccc(N6CCNCC6)cc5)c4)nc3n21]

### General Synthesis Method 54 For Intermediate Compounds

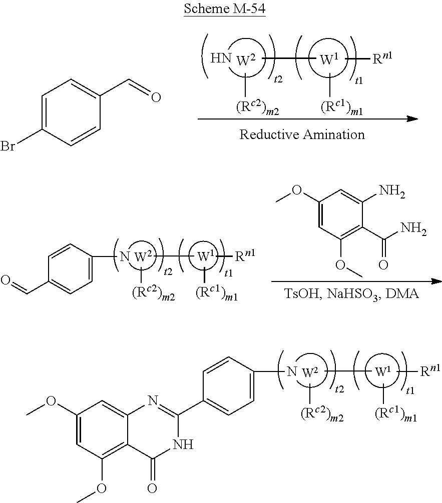

Ring W¹, ring W², t1, t2, (R^{c1})ₘ₁, and (R^{c2})ₘ₂ of the compounds in Scheme M-54 are as defined in the compound of Formula (I) of the present disclosure. Rⁿ¹ represents Boc, H, or —NHR_{g}, where R_{g} represents hydrogen or C₁₋₆ alkyl (e.g., methyl or ethyl). It should be understood that when ring W⁶-Rⁿ¹ contains additional reactive groups (such as —NH— or —NH₂) that can participate in the reaction of Scheme M-54, the reactive groups can be protected by techniques and methods known to those skilled in the art, such as using protecting groups. The removal of protecting groups can be achieved through techniques and methods known to those skilled in the art. For example, the removal of the Boc protecting group can be achieved under acidic conditions such as hydrochloric acid or trifluoroacetic acid.

### General Synthesis Method 55 For Intermediate Compounds

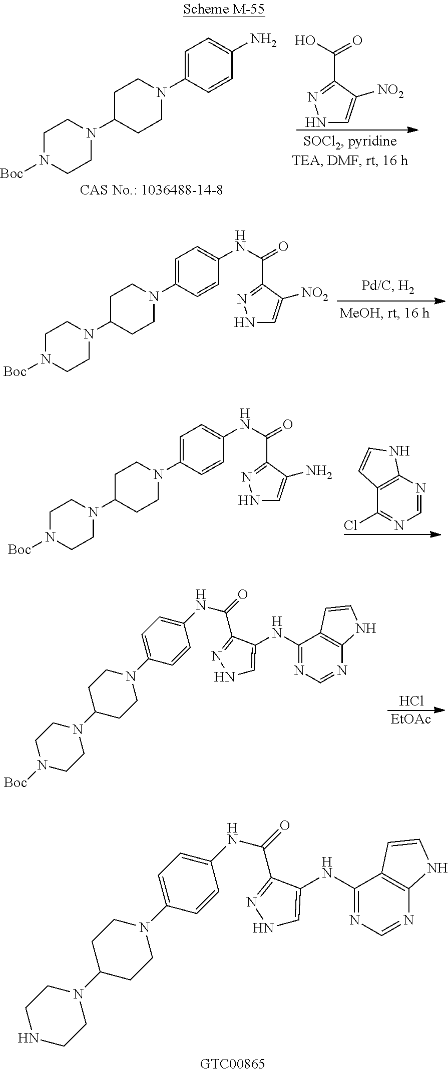

### General Synthesis Method 56 For Intermediate Compounds

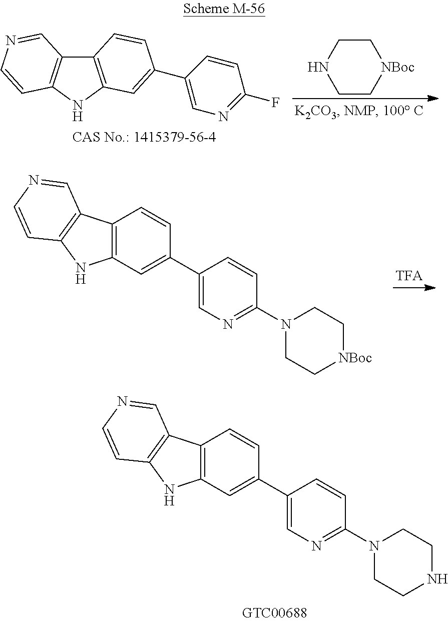 [SMILES: Fc1ccc(-c2ccc3c(c2)[nH]c2ccncc23)cn1 ; CC(C)(C)OC(=O)N1CCNCC1 ; CC(C)(C)OC(=O)N1CCN(c2ccc(-c3ccc4c(c3)[nH]c3ccncc34)cn2)CC1 ; c1cc2[nH]c3cc(-c4ccc(N5CCNCC5)nc4)ccc3c2cn1]

### General Synthesis Method 56 For Intermediate Compounds

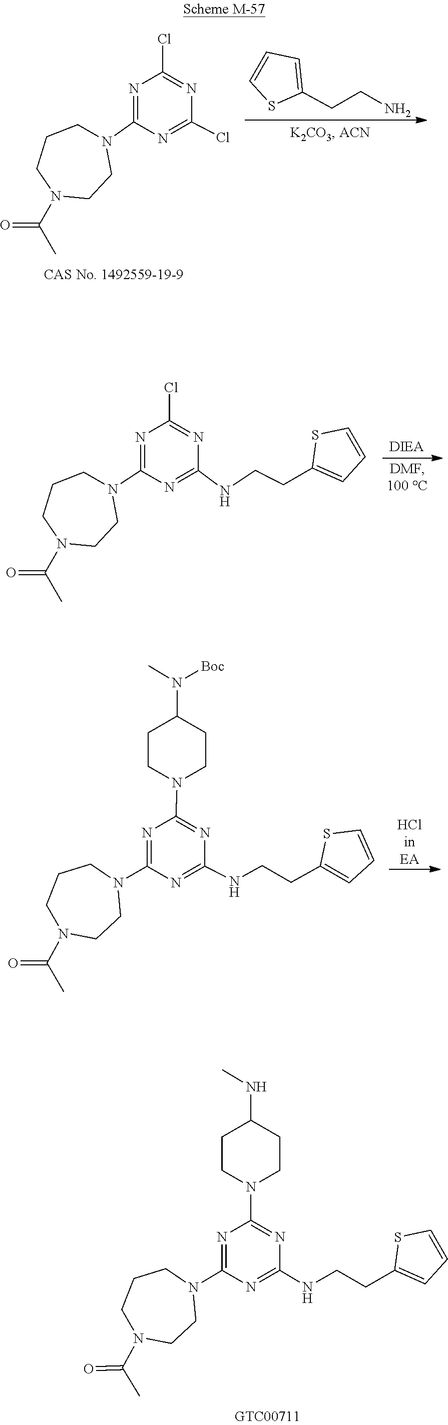 [SMILES: CC(=O)N1CCCN(c2nc(Cl)nc(Cl)n2)CC1 ; NCCc1cccs1 ; CC(=O)N1CCCN(c2nc(Cl)nc(NCCc3cccs3)n2)CC1 ; CC(=O)N1CCCN(c2nc(NCCc3cccs3)nc(N3CCC(N(C)C(=O)OC(C)(C)C)CC3)n2)CC1 ; CNC1CCN(c2nc(NCCc3cccs3)nc(N3CCCN(C(C)=O)CC3)n2)CC1]

Depending upon the target compound, the reaction substrate, reaction conditions (including reaction dosage, temperature, time, etc.), and work up, etc. in Scheme M-54 can be appropriately modified and adjusted using techniques and methods well known to those skilled in the art to obtain the desired target compound. In Scheme M-54, the reductive amination can be conventional techniques and methods known to those skilled in the art. For example, the reductive amination can be carried out at room temperature in the presence of sodium triacetoxyborohydride/1,2-dichloroethane, or sodium cyanoborohydride/1,2-dichloroethane.

### General Synthesis Method 1 for Compounds of Formula (I)

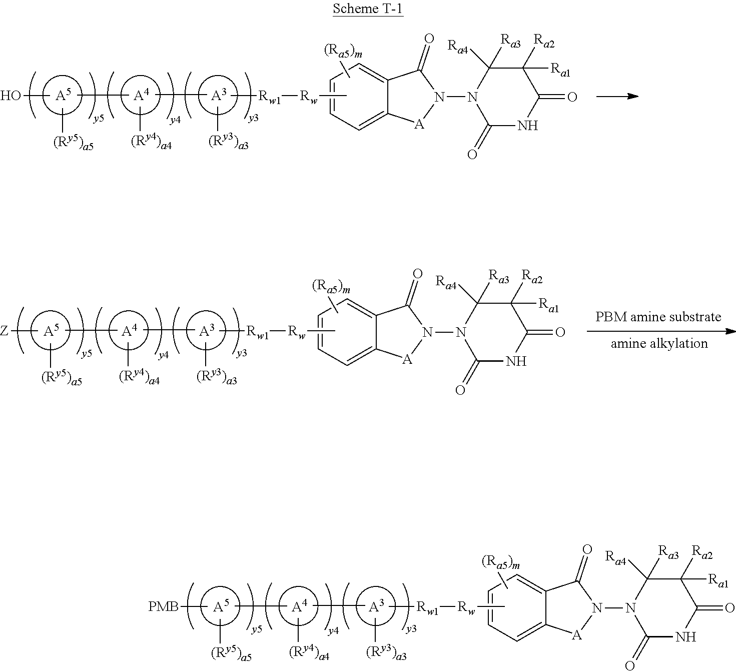

In Step 2 of Scheme T-1, the PBM amine substrate is a compound corresponding to the PBM moiety of the compound of Formula (I) of the present disclosure, containing aliphatic amine or heterocyclic amine (such as nitrogen-containing monocyclic heterocycle, nitrogen-containing bridged heterocycle, nitrogen-containing spiro heterocycle, or nitrogen-containing fused heterocycle) groups or amino compounds. The other substrate has Rₐ₁, Rₐ₂, Rₐ₃, Rₐ₄, A, (Rₐ₅)ₘ, R_{w}, R_{w1}, ring A³, ring A⁴, ring A⁵, y3, y4, y5, (R^{y3})ₐ₃, (R^{y4})ₐ₄, and (R^{y5})ₐ₅ as defined in the Formula (I) compound herein. The group Z may be Br, Cl, I, OMs, OTs, OTf, or the like. (Rₐ₅)ₘ indicates that the benzene ring to which it is attached is substituted with m Ras groups, with each Ras being the same or different and independently representing halogen, hydroxy, mercapto, nitro, amino, cyano, C₁₋₆ alkyl, halogenated C₁₋₆ alkyl, deuterated C₁₋₆ alkyl, C₁₋₆ alkoxy, deuterated C₁₋₆ alkoxy, halogenated C₁₋₆ alkoxy, C₂₋₆ alkenyl or C₂₋₆ alkynyl; m represents an integer of 0, 1, 2, 3, or 4.

Step 1:(1) When the group Z is OMs, OTs, or OTf, the alcohol substrate can undergo esterification with, for example, MsCl (methanesulfonyl chloride), TsCl (p-toluenesulfonyl chloride), or TfCl (trifluoromethanesulfonyl chloride) under basic conditions (such as TEA, DIEA) to obtain the corresponding target intermediate. Alternatively, (2) when the group Z is Br, Cl, or I, the alcohol substrate can undergo halogenation with, for example, CBr₄/PPh₃/DCM to obtain the corresponding target intermediate.

Step 2: to a solution of the amine substrate (1 equiv) and target intermediate (1.1 equiv) from step 1 in anhydrous DMF were added sodium iodide (1.0 equiv) and DIEA (3.0 equiv). The reaction mixture was stirred at room temperature to 80 °C for 0.5-24 hours. After monitoring the reaction via LCMS and confirming its completion, the reaction solution was filtered, and the filtrate was separated and purified by preparative HPLC to give the target compound.

### General Synthesis Method 2 for Compounds of Formula (I)

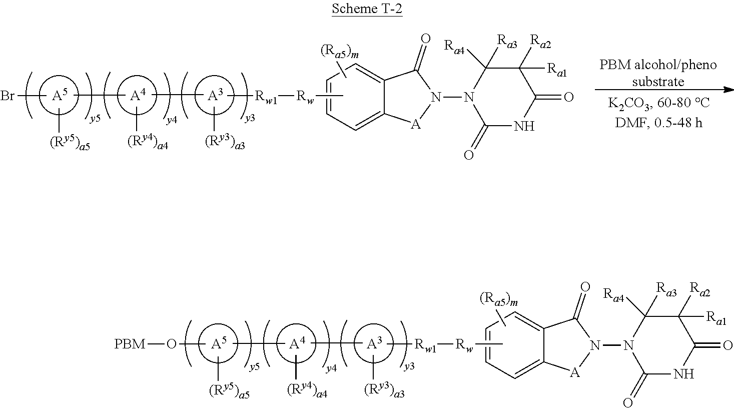

In Scheme T-2, the PBM alcohol/pheno substrate has the formula of PBM-OH. PBM, along with Rₐ₁, Rₐ₂, Rₐ₃, Rₐ₄, A, (Rₐ₅)ₘ, R_{w}, R_{w1}, ring A³, ring A⁴, ring A⁵, y3, y4, y5, (R^{y3})ₐ₃, (R^{y4})ₐ₄, and (R^{y5})ₐ₅ of the other substrate, are as defined in the compound of Formula (I) of the present disclosure.

To a solution of the PBM alcohol/pheno substrate (1 equiv) and Bromo substrate (1.2 equiv) in DMF was added K₂CO₃ (5 equiv). The reaction mixture was stirred at 50-80 °C for 0.5-48 hours. After monitoring the reaction via LCMS and confirming its completion, the reaction solution was filtered, and the filtrate was separated and purified by preparative HPLC to give the target compound.

### General Synthesis Method 3 for Compounds of Formula (I)

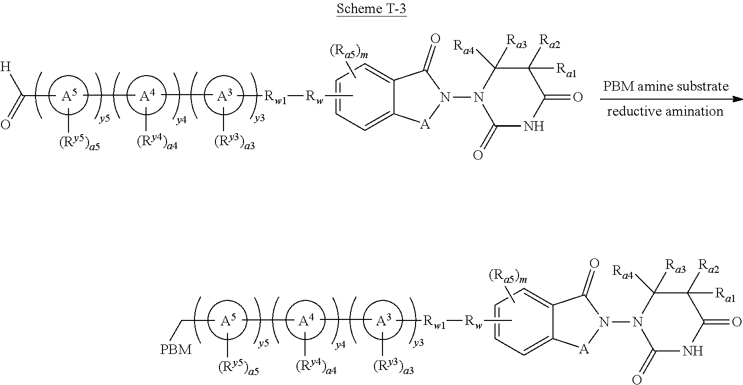

In Scheme T-3, the PBM amine substrate is a compound corresponding to the PBM moiety of the compound of Formula (I) of the present disclosure, containing aliphatic amine or heterocyclic amine (such as nitrogen-containing monocyclic heterocycle, nitrogen-containing bridged heterocycle, nitrogen-containing spiro heterocycle, or nitrogen-containing fused heterocycle) groups or amino compounds. PBM, along with Rₐ₁, Rₐ₂, Rₐ₃, Rₐ₄, A, (Rₐ₅)ₘ, R_{w}, R_{w1}, ring A³, ring A⁴, ring A⁵, y3, y4, y5, (R^{y3})ₐ₃, (R^{y4})ₐ₄, and (R^{y5}) as of the other substrate, are as defined in the compound of Formula (I) of the present disclosure. It should be understood that when the PBM amine substrate and/or aldehyde substrate contains additional reactive groups (such as —NH or —NH₂) that can participate in the reaction of Scheme T-3, the reactive groups can be protected by techniques and methods known to those skilled in the art, such as using protecting groups.

In Scheme T-3, the reductive amination can be conventional techniques and methods known to those skilled in the art. For example, the reductive amination can be conducted at a temperature ranging from room temperature to 80 °C in the presence of sodium triacetoxyborohydride and 1,2-dichloroethane. Alternatively, the reaction may be carried out using sodium borohydride in 1,4-dioxane over the same temperature range.

For example, the procedure of Scheme T-3 can be performed as follows: to a solution of the corresponding substrate aldehyde or carbonyl compound (1.0 equiv) and the PBM substrate amine (1.0 equiv) in dichloromethane (DCM) was added an appropriate amount of acetic acid. The resulting reaction mixture was reacted at room temperature for 30 minutes, followed by addition of sodium cyanoborohydride (3.0 equiv), and the reaction was continued at room temperature. After monitoring the reaction via LCMS and confirming its completion, the reaction mixture was separated by using C₁₈ reverse phase column chromatography to give the target compound.

### General Synthesis Method 4 for Compounds of Formula (I)

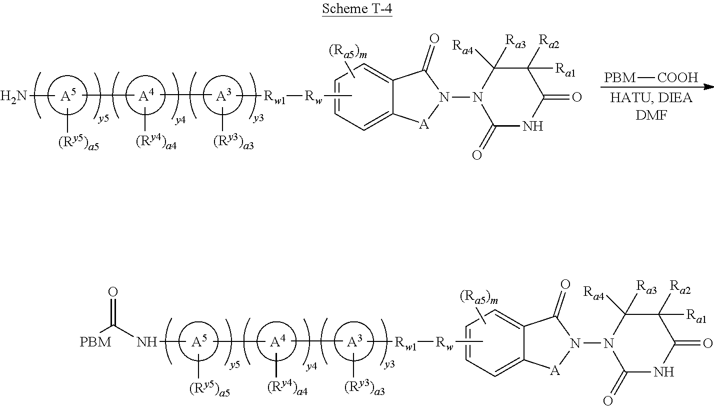

In Scheme T-4, the PBM, A, Rₐ₁, Rₐ₂, Rₐ₃, Rₐ₄, A, (Rₐ₅)ₘ, R_{w}, R_{w1}, ring A³, ring A⁴, ring A⁵, y3, y4, y5, (R^{y3})ₐ₃, (R^{y4})ₐ₄, and (R^{y5}) as of the target product are as defined herein for the Formula (I) compound. The condensation reaction in Scheme T-4 can follow conventional techniques and methods that are well-known to those skilled in the art. For example, it can be carried out using conventional condensing agents such as HOAt/EDCI, T₃P, etc., under alkaline conditions (e.g., in the presence of DMAP) at room temperature or under heating.

### General Synthesis Method 5 for Compounds of Formula (I)

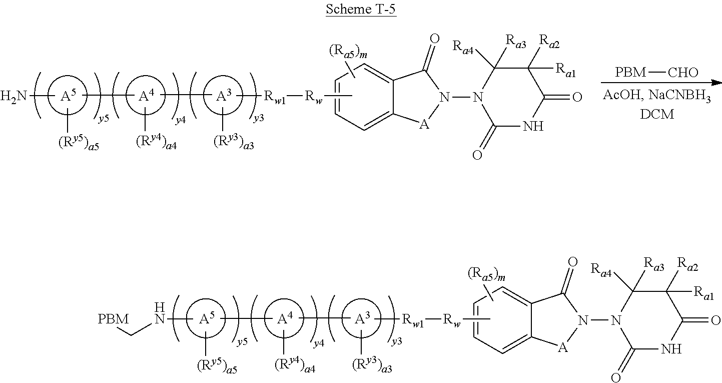

In Scheme T-5, the PBM, A, Rₐ₁, Rₐ₂, Rₐ₃, Rₐ₄, A, (Rₐ₅)ₘ, R_{w}, R_{w1}, ring A³, ring A⁴, ring A⁵, y3, y4, y5, (R^{y3})ₐ₃, (R^{y4})ₐ4, and (R^{y5}) as of the target product are as defined herein for the Formula (I) compound.

The reductive amination reaction in Scheme T-5 can follow conventional techniques and methods that are known to those skilled in the art. For example, the reductive amination reaction can be carried out at temperatures ranging from room temperature to 80 °C in the presence of sodium borohydride acetate and 1,2-dichloroethane.

For example, to a solution of the corresponding PBM aldehyde substrate (1.0 equivalent) and amine substrate (1.0 equivalent) in dichloromethane was added an appropriate amount of acetic acid. The resulting reaction mixture was allowed to react at room temperature for 30 minutes, followed by the addition of sodium cyanoborohydride (3.0 equivalents). The reaction was then continued at room temperature until completion, as monitored by LCMS. After the reaction was complete, the product was separated via C₁₈ reverse-phase column chromatography. The collected fractions were rotary evaporated to remove acetonitrile, and the resulting residue was lyophilized to give the target compound.

### General Synthesis Method 1 for Compounds of Formula (II)

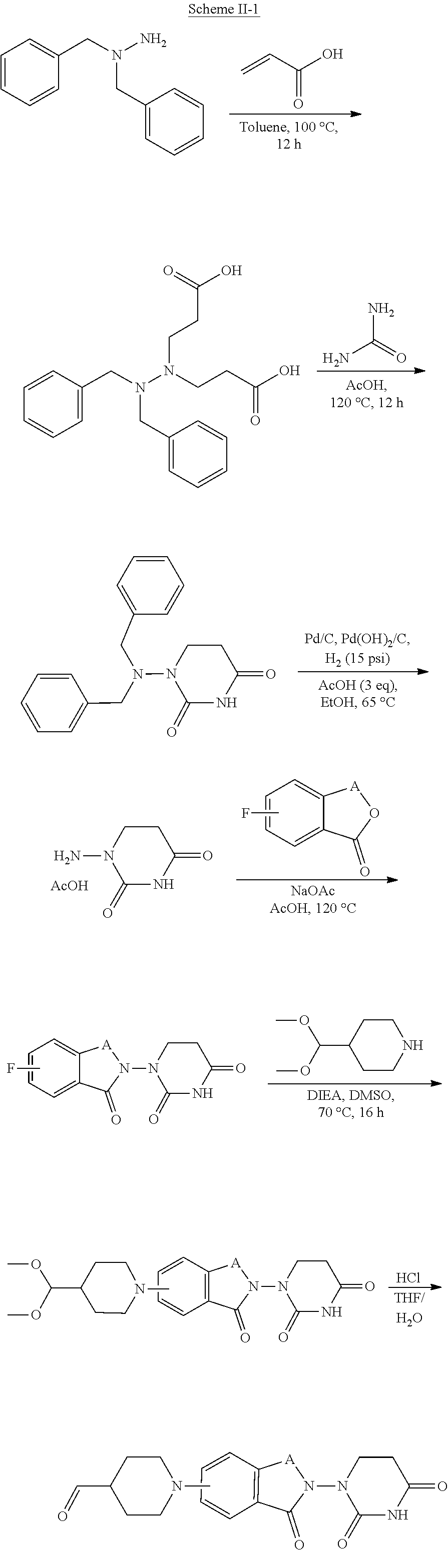

In Step 4 of Scheme II-1, A in the general structure of the fluoro substrate represents CH₂ or C(O).

### General Synthesis Method 2 for Compounds of Formula (II)

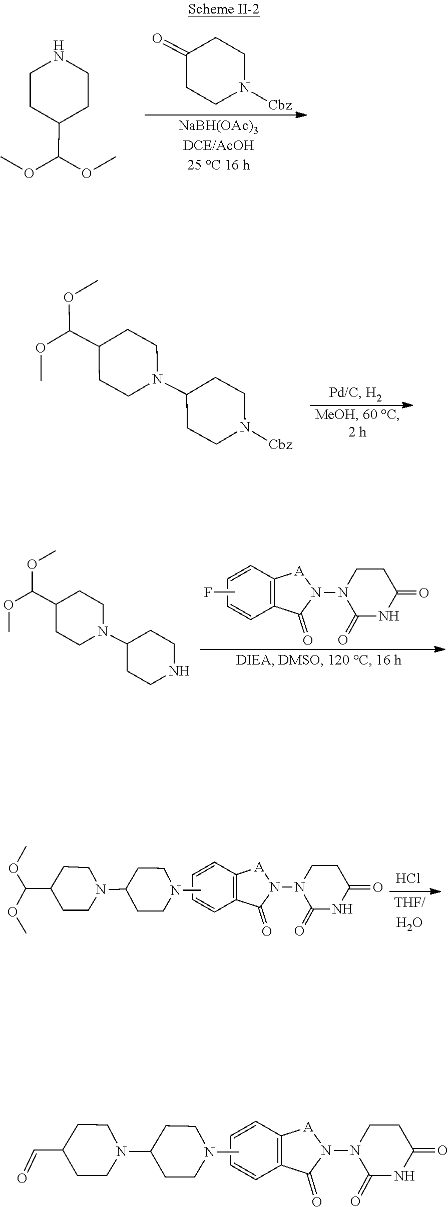

In Step 3 of Scheme II-2, A in the general structure of the fluoro substrate represents CH₂ or C(O).

### General Synthesis Method 3 for Compounds of Formula (II)

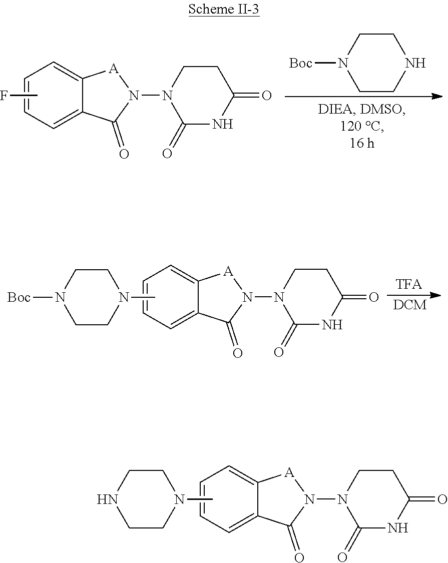

In Step 1 of Scheme II-3, A in the general structure of the fluoro substrate represents CH₂ or C(O). In Scheme II-3, the Boc protecting group can also be other protecting groups known to those skilled in the art, such as Cbz. The removal of these protecting groups can be accomplished by techniques and methods well-known in the art. For example, the Boc group can be removed under acidic conditions, such as with hydrochloric acid or trifluoroacetic acid (TFA), while the Cbz group can be removed by catalytic hydrogenation.

### General Synthesis Method 4 for Compounds of Formula (II)

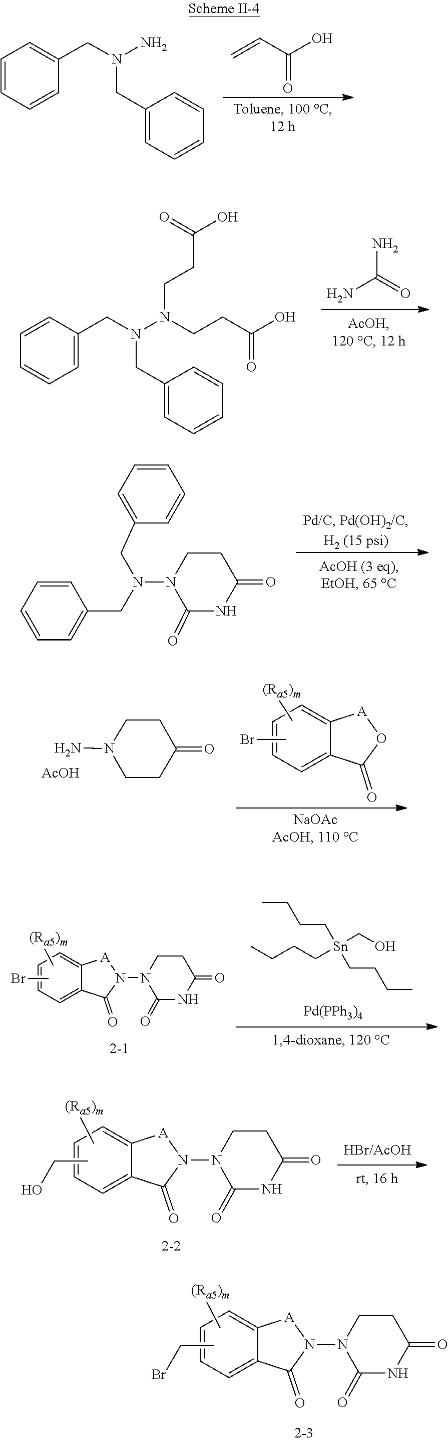

In Scheme II-4, the substituent Br on the substrate in Step 4 can be located at the 4-, 5-, 6-, or 7-position of the benzene ring of the isoindolinone moiety. Consequently, the Br substituent in the resulting intermediate 2-1, as well as the —CH₂—OH and —CH₂—Br substituents in the subsequent products 2-2 and 2-3, will correspondingly be located at the 4-, 5-, 6-, or 7-position of the benzene ring of the isoindolinone moiety. (Rₐ₅)ₘ and A are as defined in the compound of Formula (I) of the present disclosure and its various sub-embodiments.

The coupling reaction in Step 5 of Scheme II-4 can be conducted, for example, at a temperature between 40 °C and 120 °C in the presence of tetrakis(triphenylphosphine) palladium (O) and 1,4-dioxane (or DMF). The halogenation reaction in Step 6 of Scheme I-1 can be performed at room temperature using, for example, a hydrohalic acid and acetic acid. Alternatively, depending on requirements, the halogenation reaction in Step 6 can be replaced with an esterification reaction to prepare the corresponding products containing OMs, OTs, or ONs groups.

For example, the procedures for Steps 5 and 6 of Scheme II-4 can be carried out as follows:

Step 5: to a solution of the bromo substrate 2-1 (1.0 eq.) and (tributylstannyl)methanol (1.5 eq.) in DMF was added tetrakis(triphenylphosphine) palladium (O)(0.05 eq.). Under a nitrogen (N²) atmosphere, the reaction mixture was heated to 100 °C and stirred for 48 hours. Reaction completion was monitored by thin-layer chromatography (TLC). The reaction mixture was cooled to 60 °C, filtered hot to promptly remove the black solids, and the filtrate was concentrated under reduced pressure. Dichloromethane (DCM) was added to the residue, followed by filtration, to afford the solid intermediate compound 2-2.

Step 6: The solid intermediate compound 2-2 and HBr/AcOH were stirred at room temperature for 16 hours. Reaction completion was confirmed by TLC. The reaction mixture was directly concentrated to obtain the target compound.

### General Synthesis Method 5 for Compounds of Formula (II)

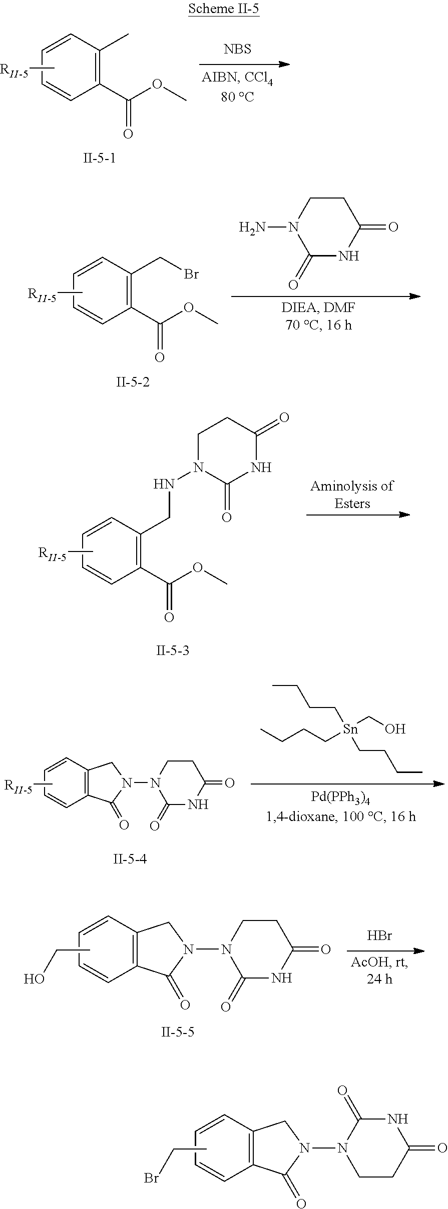

In Scheme II-5, the group R_{II-5} represents Br, Cl, or I.

The aminolysis of the ester in Step 3 of Scheme II-5 can be conducted at room temperature in the presence of CH₃ONa and methanol. Alternatively, the reaction can be performed at a temperature between 40 °C and 90 °C in the presence of CH₃OLi or Cs₂CO₃ in methanol, or DBU in THE under the same temperature range.

### General Synthesis Method 6 for Compounds of Formula (II)

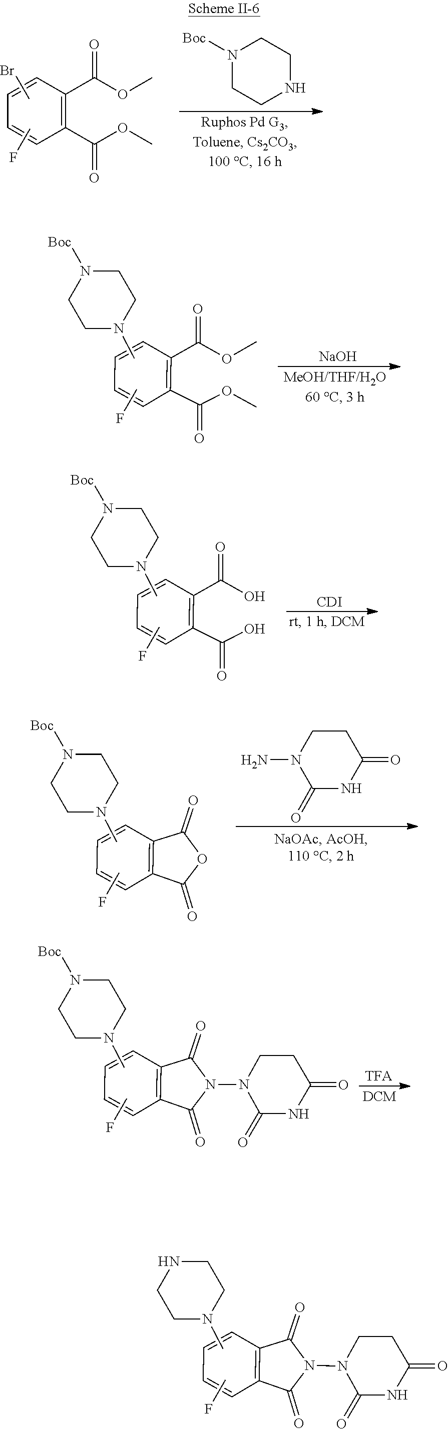

### General Synthesis Method 7 for Compounds of Formula (II)

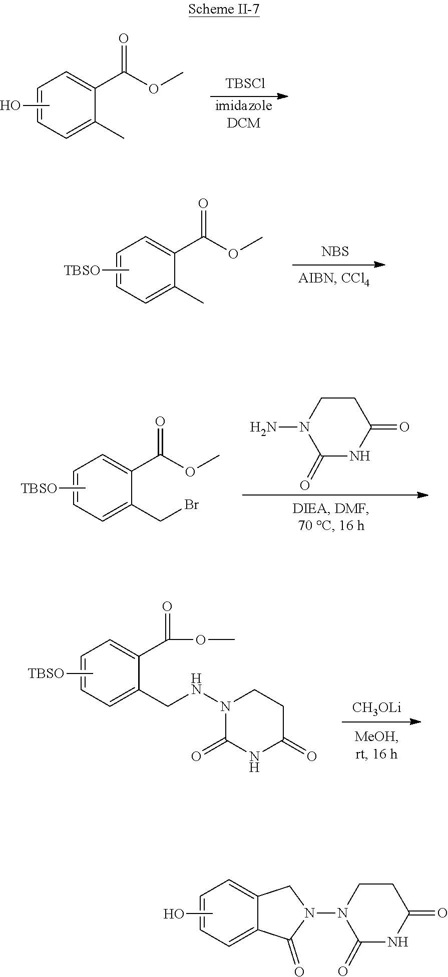

### General Synthesis Method 8 for Compounds of Formula (II)

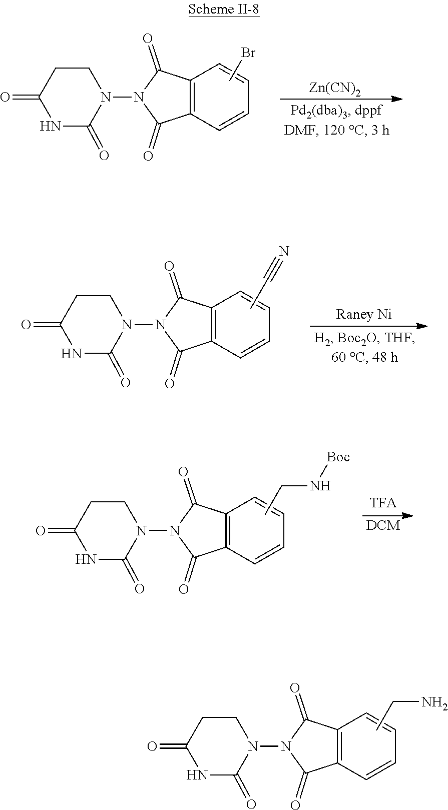

### General Synthesis Method 9 for Compounds of Formula (II)

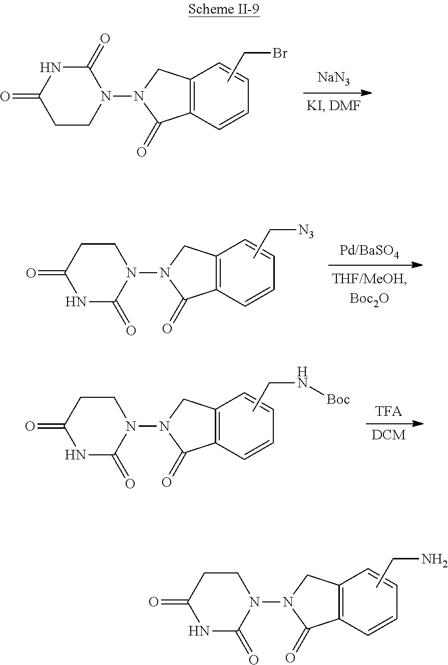

Depending upon the target compounds, the above schemes and their reaction substrates, reaction conditions (including reaction dosage, temperature, duration, etc.), work up, etc. can be appropriately modified and adjusted by techniques and methods well known to those skilled in the art to obtain the desired target compounds. The obtained target compounds can be further modified by the substituents and the like to obtain subsequent target compounds through methods well known to those skilled in the art.

### EXAMPLES

### Intermediate Example 1: Preparation of 1-((3S,4R)-3-fluoropiperidin-4-yl)-3-(4-phenoxyphenyl)-1H-pyrazolo[3,4-d]pyrimidin-4-amine

1-((3S,4R)-3-fluoropiperidin-4-yl)-3-(4-phenoxyphenyl)-1H-pyrazolo[3,4-d]pyrimidin-4-amine was prepared by referring to the method of Scheme M-1.

Step 1: to a solution of tert-butyl (3S,4S)-3-fluoro-4-hydroxypiperidine-1-carboxylate (370 mg, 1.69 mmol) in anhydrous dichloromethane were added DIEA (0.5 ml, 3.38 mmol), p-toluenesulfonyl chloride (386 mg, 2.03 mmol), and DMAP (206.16 mg, 1.69 mmol). The reaction mixture was stirred at room temperature for 18 hours, and the reaction was completed as detected by TLC. The reaction mixture was washed with H₂O (50 mL) and extracted with DCM (50 mL×3). The combined organic phases were dried over anhydrous sodium sulfate, filtered, and the filtrate was concentrated under reduced pressure. The residue was purified by column chromatography (eluent (v/v): PE/EtOAc=1/1) to give the white solid compound tert-butyl (3S,4S)-3-fluoro-4-(tosyloxy) piperidine-1-carboxylate (350 mg, yield 55%).

Step 2: to a solution of the compound tert-butyl (3S,4S)-3-fluoro-4-(tosyloxy) piperidine-1-carboxylate (350 mg, 0.94 mmol) in anhydrous DMSO (20 mL) were added 3-(4-phenoxyphenyl)-1H-pyrazolo[3,4-d]pyrimidin-4-amine (284 mg, 0.94 mmol) and K₂CO₃ (388 mg, 2.81 mmol). The reaction mixture was heated to 85 °C and stirred for 18 hours, and the reaction was completed as detected by TLC. The reaction mixture was cooled to room temperature, washed with H₂O (100 mL), and extracted with EtOAc (100 mL×3). The combined organic phases were dried over anhydrous sodium sulfate, filtered, and the filtrate was concentrated under reduced pressure. The residue was purified by column chromatography with eluent (DCM/MeOH=20/1) to give the white solid compound tert-butyl (3S,4R)-4-(4-amino-3-(4-phenoxyphenyl)-1H-pyrazolo[3,4-d]pyrimidin-1-yl)-3-fluoropiperidine-1-carboxylate (350 mg, yield 74%). LC/MS (ESI, m/z)505.3 [M+H]⁺.

Step 3: to a solution of the compound tert-butyl (3S,4R)-4-(4-amino-3-(4-phenoxyphenyl)-1H-pyrazolo[3,4-d]pyrimidin-1-yl)-3-fluoropiperidine-1-carboxylate (350 mg, 0.69 mmol) in anhydrous DCM (15 mL) was added HCl/dioxane (1.7 mL, 4M). The reaction mixture was stirred at room temperature for 1 hour, and the reaction was completed as detected by TLC. The reaction mixture was concentrated under reduced pressure to give the white solid target compound 1-((3S,4R)-3-fluoropiperidin-4-yl)-3-(4-phenoxyphenyl)-1H-pyrazolo[3,4-d]pyrimidin-4-amine (205 mg, yield 67%). ¹H NMR (400 MHZ, DMSO)_{δ 8.24} (d, J=5.6 Hz, 1H), 7.69 (d, J=8.6 Hz, 2H), 7.44 (dd, J=8.4, 7.6 Hz, 2H), 7.28-7.04 (m, 5H), 5.15-4.76 (m, 2H), 3.34 (d, J=7.3 Hz, 1H), 2.97 (d, J=12.3 Hz, 1H), 2.63 (ddd, J=15.0, 12.2, 3.5 Hz, 2H), 2.12 (dd, J=12.3, 4.1 Hz, 1H), 1.94 (d, J=12.7 Hz, 1H). ¹⁹F NMR (377 MHz, DMSO)_{δ}-185.23 (s). LC/MS (ESI, m/z) calcd for C₂₂H₂₂FN₆O⁺ [M+H]⁺: 405.2; found, 405.1.

### Intermediate Example 2: Preparation of 3-(4-phenoxyphenyl)-1-(piperidin-4-yl)-1H-pyrazolo[3,4-d]pyrimidin-4-amine hydrochloride

3-(4-phenoxyphenyl)-1-(piperidin-4-yl)-1H-pyrazolo[3,4-d]pyrimidin-4-amine hydrochloride was prepared by referring to the method of Scheme M-2.

Step 1: to a solution of triphenylphosphine (154.59 g, 574.1 mmol) in tetrahydrofuran (3 L) was added DIAD (117.54 g, 581.3 mmol) at 0 °C. After addition, the mixture was stirred for 0.5 hours. Then, to the mixture was added 2-methylpropan-2-yl 4-hydroxypiperidine-1-carboxylate (118.6 g, 589.3 mmol) at 0 °C, and the mixture was stirred for 0.5 hours. Subsequently, to the reaction solution was added 3-[4-(phenoxy)phenyl]-1H-pyrazolo[3,4-d]pyrimidin-4-amine (90 g, 296.7 mmol), and the temperature was slowly raised to 25 °C and stirred for 16 hours. TLC analysis showed the completion of the reaction. The reaction mixture was concentrated under vacuum to obtain the crude product. The crude product was purified by silica gel flash column chromatography (eluent (v/v): PE/EtOAc=I/O—1/1) to obtain the white solid compound tert-butyl 4-(4-amino-3-(4-phenoxyphenyl)-1H-pyrazolo[3,4-d]pyrimidin-1-yl) piperidine-1-carboxylate (92.0 g, 189.3 mmol, yield 63.8%). LCMS (ESI) calcd, for C₂₇H₃₁N₆O₃⁺ [M+H]⁺: 487.24; found, 487.30.

Step 2: tert-butyl 4-(4-amino-3-(4-phenoxyphenyl)-1H-pyrazolo[3,4-d]pyrimidin-1-yl) piperidine-1-carboxylate (92.0 g, 189.3 mmol) was dissolved in HCl/EtOAc (340 mL, 3 M) at 10 °C. The mixture was stirred at room temperature for 3 hours. TLC analysis showed the completion of the reaction. A large amount of white solid precipitated, which was filtered and ground with ethyl acetate/petroleum ether (1:8, 300 m¹) and filtered again. The solid was dried under high vacuum to obtain the white solid compound 4-(4-amino-3-(4-phenoxyphenyl)-1H-pyrazolo[3,4-d]pyrimidin-1-yl) piperidine hydrochloride (60 g, 154.957 mmol, yield 52%). ¹H NMR (400 MHZ, DMSO)_{δ 8.25} (s, 1H), 7.69-7.61 (m, 2H), 7.47-7.39 (m, 2H), 7.23-7.09 (m, 6H), 4.98-4.89 (m, 1H), 3.35-3.26 (m, 3H), 2.99 (t, J=12.1 Hz, 2H), 2.35-2.19 (m, 2H), 2.07-1.98 (m, 2H). LCMS (ESI) calcd, for C₂₂H₂₃N₆O⁺ [M+H]⁺: 387.19; found, 387.20.

### Intermediate Example 3: Preparation of tert-butyl 4-(2-(4-aminophenoxy)ethyl) piperazine-1-carboxylate

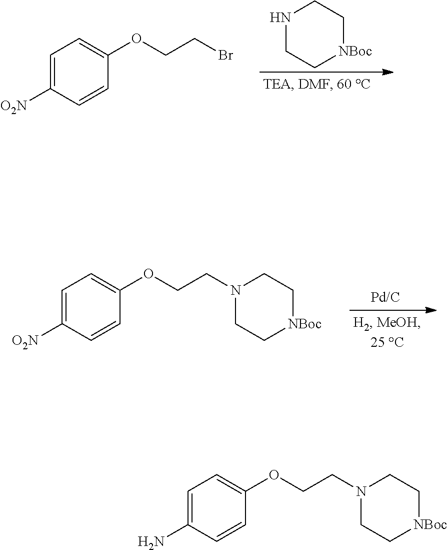 [SMILES: O=[N+]([O-])c1ccc(OCCBr)cc1 ; CC(C)(C)OC(=O)N1CCNCC1 ; CC(C)(C)OC(=O)N1CCN(CCOc2ccc([N+](=O)[O-])cc2)CC1 ; CC(C)(C)OC(=O)N1CCN(CCOc2ccc(N)cc2)CC1]

Step 1: To a solution of 1-(2-bromoethoxy)-4-nitrobenzene (4.50 g, 18.288 mmol) in N,N-dimethylformamide (100 mL) was added TEA (3.66 g, 36.576 mmol), and the reaction mixture was stirred at 60 °C for 30 minutes. TLC analysis showed the completion of the reaction. The reaction mixture was diluted with EtOAc (100 mL) and ice water (100 mL). The organic layer was separated and further washed with saturated brine (100 mL), then concentrated under vacuum. The residue was purified by silica gel column chromatography (eluent (v/v): PE/EtOAc=1/0-0/1) to obtain the colorless oily compound tert-butyl 4-(2-(4-nitrophenoxy)ethyl) piperazine-1-carboxylate (4 g, 11.383 mmol, yield 62.24%). ¹H NMR (300 MHz, CDCl₃) δ 8.19 (2H, m), 6.97 (2H, m), 4.19 (t, J=5.6; LCMS (ESI) calcd, for C₁₇H₂₆N₃O₅⁺ [M+H]⁺: 352.19 (M)_{351.18}, found, (M+H)⁺ 352.30.

Step 2: To a solution of tert-butyl 4-(2-(4-nitrophenoxy)ethyl) piperazine-1-carboxylate (4.00 g, 11.383 mmol) in MeOH (50 mL) was added Pd/C (0.12 g, 1.138 mmol, 10%), and the reaction was stirred under a hydrogen balloon at room temperature for 16 hours. LCMS analysis showed the completion of the reaction. The mixture was filtered through a celite bed and washed with methanol (20 mL). The filtrate was concentrated to obtain the off-white solid tert-butyl 4-(2-(4-aminophenoxy)ethyl) piperazine-1-carboxylate (3.1 g, yield 84.73%). LCMS (ESI) calcd, for C₁₇H₂₈N₃O₃⁺ [M+H]⁺: (M)322.21, found, (M+H)⁺ 322.20.

### Intermediate Example 4: Preparation of tert-butyl 4-(6-aminopyridazin-3-yl) piperazine-1-carboxylate

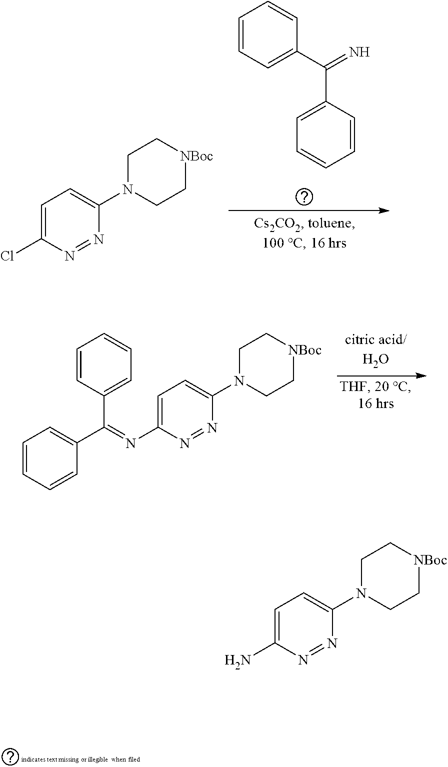

Step 1: Under argon protection, to a solution of tert-butyl 4-(6-chloropyridazin-3-yl) piperazine-1-carboxylate (3.00 g, 10.041 mmol) in toluene (60 mL) were added diphenylmethanimine (2.022 mL, 12.049 mmol), BINAP (0.06 g, 0.100 mmol), Cs₂CO₃ (6.54 g, 20.082 mmol) and Pd₂ (dba)₃ (0.46 g, 0.502 mmol). The resulting suspension was stirred at 100 °C for 16 hours. LCMS analysis showed the completion of the reaction. The reaction mixture was cooled to room temperature, then filtered through celite and washed with ethyl acetate (30 mL). The filtrate was washed with saturated brine (20 mL), dried over anhydrous magnesium sulfate, and filtered to separate the solid. The filtrate was concentrated under reduced pressure, and the resulting residue was purified by silica gel column chromatography to obtain the white solid compound tert-butyl 4-(6-((diphenylmethylene)amino)pyridazin-3-yl) piperazine-1-carboxylate (2.60 g, 5.862 mmol, yield 58.38%). LCMS (ESI) calcd, for C₂₆H₃₀N₅O₂⁺ [M+H]⁺: 443.20; found, [M+H]⁺=444.20.

Step 2: To a solution of tert-butyl 4-(6-((diphenylmethylene)amino)pyridazin-3-yl) piperazine-1-carboxylate (2.60 g, 5.862 mmol) in THF (50 mL) was added citric acid/H₂O (20 mL), and the mixture was stirred at 20 °C for 16 hours. LCMS analysis showed the completion of the reaction. The reaction mixture was concentrated under vacuum to remove THF. The resulting aqueous phase was adjusted to pH=8 with saturated NaHCO₃ and extracted with EtOAc (100 mL×3). The combined organic phases were dried over Na₂SO₄ and concentrated under reduced pressure to obtain the white solid tert-butyl 4-(6-aminopyridazin-3-yl) piperazine-1-carboxylate (1.6 g, yield 97.71%). LCMS (ESI) calcd, for C₁₃H₂₂N₅O₂⁺ [M+H]⁺: M: 280.18279.20; found, [M+H]⁺=280.20.

### Intermediate Example 5: Preparation of 6-(4-(tert-butoxycarbonyl)piperazin-1-yl)pyridazine-3-carboxylic acid

6-(4-(tert-butoxycarbonyl) piperazin-1-yl)pyridazine-3-carboxylic acid was prepared by referring to the method of Scheme M-4.

Step 1: to a solution of methyl 6-Chloropyridazine-3-carboxylate (500.00 mg, 2.897 mmol) and tert-butyl piperazine-1-carboxylate (809.45 mg, 4.346 mmol) in DMF (10.00 mL) was added DIEA (1.437 mL, 8.692 mmol), and the reaction mixture was stirred at 60 °C for 12 h. LC-MS analysis showed completion of the reaction. Water was added to the reaction mixture, which was then extracted with ethyl acetate. The organic phase was separated, washed with saturated brine, and concentrated to obtain a crude product. The crude product was purified by chromatography (eluent (v/v): PE/EA=5/1) to yield methyl 6-(4-(tert-butoxycarbonyl) piperazin-1-yl)pyridazine-3-carboxylate (900 mg, yield 75.16%) as a white solid. LCMS (ESI) calcd, for C₁₅H₂₃N⁴O₄⁺ [M+H]⁺: 323.16, found, 323.20.

Step 2: to a solution of methyl 6-(4-(tert-butoxycarbonyl) piperazin-1-yl)pyridazine-3-carboxylate (400 mg, 1.241 mmol) in a mixed solvent of 1,4-dioxane and water (1/1; 10 mL) was added Lithium hydroxide (260.32 mg, 6.204 mmol), and the reaction mixture was stirred at 60 °C for 12 h. LC-MS analysis showed completion of the reaction. The reaction mixture was concentrated under reduced pressure to remove the organic phase. The remaining aqueous phase was adjusted to pH=6 with concentrated hydrochloric acid and then extracted with ethyl acetate. The organic phase was separated, concentrated under reduced pressure to obtain 6-(4-(tert-butoxycarbonyl) piperazin-1-yl)pyridazine-3-carboxylic acid (300 mg, yield 76.06%) as a white solid, which can be used for the next step without further purification. LCMS (ESI) calcd, for C₁₄H₂₁N⁴O₄⁺ [M+H]⁺: 309.15, found, 309.20.

### Intermediate Example 6: Preparation of N-((1r,4r)-4-(3-chloro-4-cyanophenoxy)cyclohexyl)-6-(piperazin-1-yl)pyridazine-3-carboxamide

N-((1r,4r)-4-(3-chloro-4-cyanophenoxy)cyclohexyl)-6-(piperazin-1-yl)pyridazine-3-carboxamide was prepared by referring to the method of Scheme M-5.

To a solution of 6-(4-(tert-butoxycarbonyl) piperazin-1-yl)pyridazine-3-carboxylic acid (300 mg, 1.135 mmol) prepared from Intermediate Example 5 and 4-(((1r,4r)-4-aminocyclohexyl)oxy)-2-chlorobenzonitrile (326.61 mg, 1.135 mmol) in DMF (5.00 mL) were added HATU (647.40 mg, 1.703 mmol) and DIEA (0.938 mL, 5.676 mmol), and the resulting reaction mixture was stirred at room temperature for 30 minutes. LC-MS analysis showed completion of the reaction. Water was added to the reaction mixture, which was then extracted with ethyl acetate. The organic phase was separated, washed with saturated brine, and concentrated to obtain a crude product. The crude product was purified by chromatography (eluent (v/v): DCM/MeOH=100/1) to yield the white solid compound tert-butyl 4-(6-(((1r,4r)-4-(3-chloro-4-cyanophenoxy)cyclohexyl) carbamoyl)pyridazin-3-yl) piperazine-1-carboxylate (250 mg, yield 39.49%). LCMS (ESI) calcd, for C₂₇H₃₄ClN₆O₄⁺ [M+H]⁺: 541.23, found, 541.30.

Tert-butyl 4-(6-(((1r,4r)-4-(3-chloro-4-cyanophenoxy)cyclohexyl) carbamoyl)pyridazin-3-yl) piperazine-1-carboxylate (250 mg, 0.462 mmol) was dissolved in a mixed solvent of DMF (5.00 mL) and TFA (1.00 mL). The reaction mixture was stirred at room temperature for 3 h. LC-MS analysis showed completion of the reaction. The reaction mixture was concentrated under reduced pressure to remove the organic phase. The remaining aqueous phase was adjusted to pH=8 with saturated NaHCO₃ solution and then extracted with ethyl acetate. The organic phase was separated, concentrated under reduced pressure to obtain N-((1r,4r)-4-(3-chloro-4-cyanophenoxy)cyclohexyl)-6-(piperazin-1-yl)pyridazine-3-carboxamide (170 mg, yield 75.10%) as a white solid, which can be used for the next step without further purification. LCMS (ESI) calcd, for C₂₂H₂₆ClN₆O₂ [M+H]⁺: 441.17, found, 441.20.

### Intermediate Example 7: Preparation of 5-chloro-N²-(2-isopropoxy-5-methyl-4-(piperazin-1-yl)phenyl)-N⁴-(2-(isopropylsulfonyl)phenyl)pyrimidine-2,4-diamine (GT-D-86)

5-chloro-N²-(2-isopropoxy-5-methyl-4-(piperazin-1-yl)phenyl)-N⁴-(2-(isopropylsulfonyl)phenyl)pyrimidine-2,4-diamine (GT-D-86) was prepared by referring to Scheme M-8 (brown solid, 2.46 g). ¹H NMR (400 MHZ, DMSO)_{δ 9.51} (s, 1H), 9.14 (s, 2H), 8.43 (d, J=8.3 Hz, 1H), 8.26 (s, 1H), 8.20 (s, 1H), 7.85 (dd, J=7.9, 1.2 Hz, 1H), 7.63 (t, J=7.7 Hz, 1H), 7.51 (s, 1H), 7.37 (t, J=7.5 Hz, 1H), 6.70 (s, 1H), 4.56 (dd, J=12.0, 6.0 Hz, 1H), 3.23 (s, 4H), 3.04 (d, J=4.3 Hz, 4H), 2.09 (s, 3H), 1.19 (dd, J=24.2, 6.4 Hz, 12H). LCMS (ESI) calcd, for C₂₇H₃₆ClN₆O₃S⁺ [M+H]⁺: 559.2; found, 559.1.

### Intermediate Example 8: Preparation of 5-chloro-N²-(2-isopropoxy-5-methyl-4-(4-(methylamino) piperidin-1-yl)phenyl)-N⁴-(2-(isopropylsulfonyl)phenyl)pyrimidine-2,4-diamine (GT-D-87)

5-chloro-N²-(2-isopropoxy-5-methyl-4-(4-(methylamino) piperidin-1-yl)phenyl)-N⁴-(2-(isopropylsulfonyl)phenyl)pyrimidine-2,4-diamine (GT-D-87) was prepared by referring to Scheme M-8 (white solid, 1.2 g). ¹H NMR (400 MHZ, DMSO)_{δ 9.93} (s, 1H), 9.19 (s, 2H), 8.93 (s, 1H), 8.42 (s, 1H), 8.20 (s, 1H), 7.90 (s, 1H), 7.73 (t, J=7.7 Hz, 1H), 7.51 (t, J=7.7 Hz, 1H), 7.39 (s, 1H), 6.81 (s, 1H), 4.71-4.36 (m, 1H), 3.48 (dt, J=13.4, 6.7 Hz, 1H), 2.57 (t, J=5.3 Hz, 3H), 2.13 (s, 2H), 2.04 (s, 3H), 1.82 (s, 2H), 1.24 (d, J=6.0 Hz, 6H), 1.14 (d, J=6.8 Hz, 6H). LCMS (ESI) calcd, for C₂₉H₄₀ClN₆O₃S⁺ [M+H]⁺: 587.3; found, 587.3.

### Intermediate Example 9: Preparation of 5-chloro-N²-(2-isopropoxy-5-methyl-4-(4-(piperazin-1-yl) piperidin-1-yl)phenyl)-N⁴-(2-(isopropylsulfonyl)phenyl)pyrimidine-2,4-diamine (GT-D-88)

5-chloro-N²-(2-isopropoxy-5-methyl-4-(4-(piperazin-1-yl) piperidin-1-yl)phenyl)-N⁴-(2-(isopropylsulfonyl)phenyl)pyrimidine-2,4-diamine (GT-D-88) was prepared by referring to Scheme M-8 (brown solid, 1.2 g). ¹H NMR (400 MHZ, DMSO)_{δ 9.47} (s, 1H), 8.46 (d, J=7.9 Hz, 1H), 8.24 (s, 1H), 8.09 (s, 1H), 7.83 (d, J=7.9 Hz, 1H), 7.61 (t, J=7.8 Hz, 1H), 7.49 (s, 1H), 7.35 (t, J=7.7 Hz, 1H), 6.70 (s, 1H), 4.60-4.42 (m, 1H), 3.54-3.36 (m, 8H), 3.26-3.06 (m, 4H), 2.66 (t, J=11.1 Hz, 2H), 2.15 (d, J=11.0 Hz, 1H), 2.09 (s, 3H), 1.79 (s, 2H), 1.26 (dd, J=13.1, 7.2 Hz, 3H), 1.22-1.09 (m, 9H). LCMS (ESI) calcd, for C₃₂H₄₅ClN₇O₃S⁺ [M+H]⁺: 642.3; found, 642.3.

### Intermediate Example 10: Preparation of (2-((5-chloro-2-((2-methoxy-4-(piperazin-1-yl)phenyl)amino)pyrimidin-4-yl)amino)phenyl)dimethylphosphine oxide (GT-D-39)

(2-((5-chloro-2-((2-methoxy-4-(piperazin-1-yl)phenyl)amino)pyrimidin-4-yl)amino)phenyl)dimethylphosphine oxide (GT-D-39) was prepared by referring to Scheme M-8 (yellow solid, 7.9 g). ¹H NMR (400 MHZ, DMSO)_{δ 11.57} (s, 1H), 9.55 (s, 1H), 9.17 (s, 2H), 8.76 (s, 1H), 8.44 (s, 1H), 8.17 (s, 1H), 7.59 (dd, J=13.3, 7.7 Hz, 1H), 7.50-7.30 (m, 2H), 7.17 (t, J=7.3 Hz, 1H), 6.73 (d, J=2.1 Hz, 1H), 6.56 (dd, J=8.6, 2.2 Hz, 1H), 3.79 (s, 3H), 3.49-3.36 (m, 4H), 3.26 (m, 4H), 1.80 (s, 3H), 1.77 (s, 3H). LCMS (ESI) calcd, for C₂₃H₂₉ClN₆O₂P⁺ [M+H]⁺: 487.2; found, 487.2.

### Intermediate Example 11: Preparation of 2-((2-((2-methoxy-4-(piperazin-1-yl)phenyl)amino)-5-(trifluoromethyl)pyridin-4-yl)amino)-N-methylbenzamide (GT-M-10)

2-((2-((2-methoxy-4-(piperazin-1-yl)phenyl)amino)-5-(trifluoromethyl)pyridin-4-yl)amino)-N-methylbenzamide (GT-M-10) was prepared by referring to Scheme M-8 (white solid, 1.87 g). ¹H NMR (400 MHz, MeOD) δ 8.01 (s, 1H), 7.73 (d, J=7.9 Hz, 1H), 7.59 (d, J=3.8 Hz, 2H), 7.36 (dt, J=8.4, 4.1 Hz, 1H), 7.18 (d, J=8.6 Hz, 1H), 6.79 (d, J=2.1 Hz, 1H), 6.68 (dd, J=8.7, 2.2 Hz, 1H), 6.48 (s, 1H), 3.87 (s, 3H), 3.66 (s, 4H), 3.56-3.48 (m, 4H), 3.43-3.37 (m, 4H), 2.91 (d, J=13.7 Hz, 3H). LCMS (ESI) calcd, for C₂₅H₂₈F₃N₆O₂⁺ [M+H]⁺: 501.2; found, 501.2.

### Intermediate Example 12: Preparation of 2-((2-((4-(4-aminopiperidin-1-yl)-2-methoxyphenyl)amino)-5-(trifluoromethyl)pyridin-4-yl)amino)-N-methylbenzamide (GT-M-11)

2-((2-((4-(4-aminopiperidin-1-yl)-2-methoxyphenyl)amino)-5-(trifluoromethyl)pyridin-4-yl)amino)-N-methylbenzamide (GT-M-11) was prepared by referring to Scheme M-8 (brown solid, 0.4 g). ¹H NMR (400 MHZ, DMSO) § 10.13 (s, 1H), 8.63 (d, J=4.2 Hz, 1H), 8.27 (s, 1H), 8.14 (s, 1H), 7.74-7.60 (m, 1H), 7.44 (dd, J=6.3, 1.3 Hz, 2H), 7.36 (d, J=8.7 Hz, 1H), 7.08-7.00 (m, 1H), 6.57 (d, J=2.5 Hz, 1H), 6.51 (s, 1H), 6.43 (dd, J=8.8, 2.5 Hz, 1H), 3.77 (s, 3H), 3.57 (d, J=12.6 Hz, 3H), 2.75 (d, J=3.7 Hz, 4H). LCMS (ESI) calcd. for C₂₆H₃₀F₃N₆O₂⁺ [M+H]⁺: 515.2; found, 515.2.

### Intermediate Example 13: Preparation of 2-((2-((2-methoxy-4-(4-(piperazin-1-yl) piperidin-1-yl)phenyl)amino)-5-(trifluoromethyl)pyridin-4-yl)amino)-N-methylbenzamide (GT-M-12)

2-((2-((2-methoxy-4-(4-(piperazin-1-yl) piperidin-1-yl)phenyl)amino)-5-(trifluoromethyl)pyridin-4-yl)amino)-N-methylbenzamide (GT-M-12) was prepared by referring to Scheme M-8 (purple solid, 0.34 g). 1H NMR (400 MHZ, DMSO)_{δ 10.60} (s, 1H), 10.10 (s, 1H), 9.99 (s, 1H), 9.75 (s, 1H), 8.82 (d, J=4.6 Hz, 1H), 8.14 (s, 1H), 7.77 (d, J=7.7 Hz, 1H), 7.57 (d, J=3.2 Hz, 2H), 7.33-7.26 (m, 1H), 7.19 (s, 1H), 6.81 (s, 1H), 6.68 (s, 1H), 6.54 (s, 1H), 3.78 (d, J=26.5 Hz, 15H), 3.53 (s, 9H), 2.84 (s, 2H), 2.76 (d, J=4.5 Hz, 3H), 2.22 (d, J=10.6 Hz, 2H), 1.87 (d, J=9.5 Hz, 2H), 1.23 (s, 2H). LCMS (ESI) calcd, for C₃₀H₃₇F₃N₇O₂⁺ [M+H]⁺: 584.3; found, 584.0.

### Intermediate Example 14: Preparation of 2-((2-((2-methoxy-4-(piperazin-1-yl)phenyl)amino)-5-(trifluoromethyl)pyridin-4-yl)amino)-N-methylbenzamide (GT-M-171)

2-((2-((2-methoxy-4-(piperazin-1-yl)phenyl)amino)-5-(trifluoromethyl)pyridin-4-yl)amino)-N-methylbenzamide (GT-M-171) was prepared by referring to Scheme M-8 (yellow solid, 20.5 g). ¹H NMR (400 MHz, DMSO) & 10.39 (s, 1H), 9.20 (s, 1H), 8.80 (s, 2H), 8.69 (d, J=4.6 Hz, 1H), 8.13 (s, 1H), 7.73 (d, J=7.7 Hz, 1H), 7.53 (s, 2H), 7.35 (d, J=8.6 Hz, 1H), 7.20 (s, 1H), 6.70 (d, J=2.2 Hz, 1H), 6.59-6.49 (m, 2H), 3.80 (s, 3H), 3.36 (d, J=5.2 Hz, 4H), 3.25 (s, 4H), 2.76 (d, J=4.5 Hz, 3H). LCMS (ESI) calcd, for C₂₅H₂₈F₃N₆₀₂⁺ [M+H]⁺: 501.2; found, 501.3.

### Intermediate Example 15: Preparation of 2-((2-((2-methoxy-4-(4-(methylamino) piperidin-1-yl)phenyl)amino)-5-(trifluoromethyl)pyridin-4-yl)amino)-N-methylbenzamide (GT-M-172)

2-((2-((2-methoxy-4-(4-(methylamino) piperidin-1-yl)phenyl)amino)-5-(trifluoromethyl)pyridin-4-yl)amino)-N-methylbenzamide (GT-M-172) was prepared by referring to Scheme M-8 (white solid, 1.8 g). 1H NMR (400 MHZ, DMSO)_{δ 10.60} (s, 1H), 9.98 (s, 1H), 9.38 (s, 2H), 8.83 (d, J=4.5 Hz, 1H), 8.17 (s, 1H), 7.78 (d, J=7.8 Hz, 1H), 7.57 (d, J=3.9 Hz, 2H), 7.34-7.15 (m, 2H), 6.87 (d, J=54.9 Hz, 2H), 6.59 (s, 1H), 3.82 (s, 5H), 3.21 (s, 1H), 2.98 (s, 2H), 2.69 (t, J=41.5 Hz, 4H), 2.54 (t, J=5.3 Hz, 3H), 2.17 (s, 2H), 1.84 (s, 2H). LCMS (ESI) calcd, for C₂₇H₃₂F₃N₆₀₂⁺ [M+H]⁺: 529.3; found, 529.3.

### Intermediate Example 16: Preparation of N-methyl-N-(3-(((2-((4-(piperazin-1-yl)phenyl)amino)-5-(trifluoromethyl)pyrimidin-4-yl)amino)methyl) pyrazin-2-yl)methanesulfonamide (GT-M-151)

N-methyl-N-(3-(((2-((4-(piperazin-1-yl)phenyl)amino)-5-(trifluoromethyl)pyrimidin-4-yl)amino)methyl) pyrazin-2-yl)methanesulfonamide (GT-M-151) was prepared by referring to Scheme M-11 (white solid, 1.7 g). ¹H NMR (400 MHZ, MeOD)_{δ 8.60} (s, 1H), 8.56 (s, 1H), 8.30 (s, 1H), 7.41 (d, J=8.3 Hz, 2H), 7.20 (d, J=8.6 Hz, 2H), 5.13 (s, 2H), 3.59 (d, J=4.7 Hz, 4H), 3.49 (s, 4H), 3.20 (d, J=16.1 Hz, 3H), 3.13 (s, 3H). LCMS (ESI) calcd, for C₂₂H₂₇F₃N₉O₂S⁺ [M+H]⁺: 538.20; found, 538.3.

### Intermediate Example 17: Preparation of N-methyl-N-(3-(((2-((4-(piperazin-1-yl)phenyl)amino)-5-(trifluoromethyl)pyrimidin-4-yl)amino)methyl)phenyl)methanesulfonamide (GT-M-152)

N-methyl-N-(3-(((2-((4-(piperazin-1-yl)phenyl)amino)-5-(trifluoromethyl)pyrimidin-4-yl)amino)methyl)phenyl)methanesulfonamide (GT-M-152) was prepared by referring to Scheme M-11 (brown solid, 1.2 g). ¹H NMR (400 MHz, DMSO) § 10.92 (s, 1H), 9.52 (s, 2H), 9.06 (s, 1H), 8.48 (s, 1H), 7.43-7.25 (m, 6H), 7.18 (s, 1H), 6.97 (d, J=8.3 Hz, 2H), 4.64 (d, J=5.2 Hz, 2H), 3.37 (s, 4H), 3.20 (s, 4H), 3.15 (s, 3H), 2.87 (s, 3H). LCMS (ESI) calcd, for C₂₄H₂₉F₃N₇O₂S⁺ [M+H]⁺: 536.2; found, 536.7.

### Intermediate Example 18: Preparation of N-methyl-N-(3-(((2-((4-(4-(methylamino) piperidin-1-yl)phenyl)amino)-5-(trifluoromethyl)pyrimidin-4-yl)amino)methyl)phenyl)methanesulfonamide (GT-M-153)

N-methyl-N-(3-(((2-((4-(4-(methylamino) piperidin-1-yl)phenyl)amino)-5-(trifluoromethyl)pyrimidin-4-yl)amino)methyl)phenyl)methanesulfonamide (GT-M-153) was prepared by referring to Scheme M-11 (white solid, 1.069 g). ¹H NMR (400 MHZ, MeOD)_{δ 8.36} (d, J=22.7 Hz, 1H), 7.77 (t, J=8.4 Hz, 2H), 7.55 (d, J=8.5 Hz, 2H), 7.47 (t, J=7.9 Hz, 1H), 7.35 (d, J=7.8 Hz, 1H), 7.26 (s, 2H), 7.14 (s, 1H), 4.75 (s, 2H), 3.82 (dd, J=24.4, 12.5 Hz, 4H), 3.63 (d, J=11.4 Hz, 1H), 3.27 (d, J=8.0 Hz, 3H), 2.89 (d, J=18.3 Hz, 3H), 2.81 (s, 3H), 2.50 (d, J=13.0 Hz, 2H), 2.37 (d, J=10.7 Hz, 2H). LCMS (ESI) calcd, for C₂₆H₃₃F₃N₇O₂S⁺ [M+H]⁺: 564.2; found, 564.1.

### Intermediate Example 19: Preparation of N-methyl-N-(3-(((2-((4-(4-(methylamino) piperidin-1-yl)phenyl)amino)-5-(trifluoromethyl)pyrimidin-4-yl)amino)methyl) pyrazin-2-yl)methanesulfonamide (GT-M-174)

N-methyl-N-(3-(((2-((4-(4-(methylamino) piperidin-1-yl)phenyl)amino)-5-(trifluoromethyl)pyrimidin-4-yl)amino)methyl) pyrazin-2-yl)methanesulfonamide (GT-M-174) was prepared by referring to Scheme M-11 (white solid, 707 mg). ¹H NMR (400 MHZ, MeOD)_{δ 8.58} (dd, J=17.7, 2.3 Hz, 2H), 8.39 (s, 1H), 7.76 (dd, J=44.2, 9.0 Hz, 4H), 5.16 (s, 2H), 3.83 (d, J=13.7 Hz, 4H), 3.27 (s, 3H), 3.16 (s, 3H), 2.80 (s, 3H), 2.55-2.33 (m, 4H). LCMS (ESI) calcd, for C₂₄H₃₁F₃N₉O₂S⁺ [M+H]⁺: 566.2; found, 566.3.

### Intermediate Example 20: Preparation of N-methyl-N-(3-(((2-((4-(4-(piperazin-1-yl) piperidin-1-yl)phenyl)amino)-5-(trifluoromethyl)pyrimidin-4-yl)amino)methyl) pyrazin-2-yl)methanesulfonamide (GT-M-175)

N-methyl-N-(3-(((2-((4-(4-(piperazin-1-yl)piperidin-1-yl)phenyl)amino)-5-(trifluoromethyl)pyrimidin-4-yl)amino)methyl)pyrazin-2-yl)methanesulfonamide (GT-M-175) was prepared by referring to Scheme M-11 (brown solid, 560 mg). ¹H NMR (400 MHz, MeOD) δ 8.58 (dd, J=18.6, 2.4 Hz, 1H), 8.37 (s, 1H), 7.66 (s, 2H), 5.15 (s, 1H), 4.00-3.60 (m, 6H), 3.26 (s, 2H), 3.16 (s, 2H), 2.54 (dd, J=43.4, 11.6 Hz, 2H). LCMS (ESI) calcd. for C₂₇H₃₆F₃N₁₀O₂S⁺ [M+H]⁺: 621.3; found, 621.4.

### Intermediate Example 21: Preparation of N₄-(3-(methylsulfonyl)benzyl)-N₂-(4-(piperazin-1-yl)phenyl)-5-(trifluoromethyl)pyrimidine-2,4-diamine (GT-M-191)

N₄-(3-(methylsulfonyl)benzyl)-N₂-(4-(piperazin-1-yl)phenyl)-5-(trifluoromethyl)pyrimidine-2,4-diamine (GT-M-191) was prepared by referring to Scheme M-11 (purple solid, 500 mg). ¹H NMR (400 MHz, DMSO) δ 9.78 (s, 1H), 8.62 (s, 3H), 8.26 (s, 1H), 7.90 (s, 1H), 7.81 (d, J=7.1 Hz, 1H), 7.61 (t, J=7.6 Hz, 2H), 7.31 (d, J=7.9 Hz, 2H), 6.91 (d, J=8.7 Hz, 2H), 4.71 (d, J=5.5 Hz, 2H), 3.35-3.20 (m, 8H), 3.16 (d, J=11.3 Hz, 3H). LCMS (ESI) calcd, for C₂₃H₂₆F₃N₆O₂S⁺ [M+H]⁺: 507.2; found, 507.4.

### Intermediate Example 22: Preparation of N-methyl-N-(3-(((2-((4-(piperidin-4-yl)phenyl)amino)-5-(trifluoromethyl)pyrimidin-4-yl)amino)methyl)phenyl)methanesulfonamide (GT-M-193)

N-methyl-N-(3-(((2-((4-(piperidin-4-yl)phenyl)amino)-5-(trifluoromethyl)pyrimidin-4-yl)amino)methyl)phenyl)methanesulfonamide (GT-M-193) was prepared by referring to Scheme M-12 (white solid, 0.87 g). ¹H NMR (400 MHz, DMSO) δ 9.75 (s, 1H), 8.63 (s, 1H), 8.38 (s, 1H), 8.25 (s, 1H), 7.98 (s, 1H), 7.51 (d, J=8.0 Hz, 2H), 7.41-7.31 (m, 2H), 7.24 (dd, J=16.3, 7.7 Hz, 2H), 7.09 (d, J=8.4 Hz, 2H), 4.66 (d, J=5.6 Hz, 2H), 3.37 (d, J=12.2 Hz, 2H), 3.15 (s, 3H), 3.00 (q, J=11.9 Hz, 2H), 2.86 (s, 3H), 2.77 (t, J=12.0 Hz, 1H), 1.92 (d, J=13.5 Hz, 2H), 1.73 (dd, J=22.9, 12.5 Hz, 2H). LCMS (ESI) calcd, for C₂₅H₃₀F₃N₆O₂S⁺ [M+H]⁺: 535.2; found, 535.3.

### Intermediate Example 23: Preparation of 4-(((4-(5-chloro-2-(piperidin-4-ylamino)pyridin-4-yl) thiazol-2-yl)amino)methyl)tetrahydro-2H-pyran-4-carbonitrile (GT-M-158)

4-(((4-(5-chloro-2-(piperidin-4-ylamino)pyridin-4-yl) thiazol-2-yl)amino)methyl)tetrahydro-2H-pyran-4-carbonitrile (GT-M-158) was prepared by referring to Scheme M-14 (yellow solid, 900 mg). ¹H NMR (400 MHz, DMSO) δ 8.52 (s, 1H), 8.34 (s, 1H), 8.12 (t, J=6.2 Hz, 1H), 8.02 (s, 1H), 7.39 (s, 1H), 7.11 (s, 1H), 6.99 (s, 1H), 3.92 (s, 2H), 3.68 (d, J=6.1 Hz, 2H), 3.49 (t, J=11.3 Hz, 3H), 3.31 (d, J=12.7 Hz, 2H), 3.03 (d, J=10.3 Hz, 2H), 2.07 (d, J=10.8 Hz, 2H), 1.88 (d, J=13.4 Hz, 2H), 1.77-1.59 (m, 4H). LCMS (ESI) calcd. for C₂₀H₂₅ClN₆OS⁺ [M+H]⁺: 433.16; found, 433.1.

### Intermediate Example 24: Preparation of 4-(((5′-chloro-2′-(piperidin-4-ylamino)-[2,4′-bipyridin]-6-yl)amino)methyl)tetrahydro-2H-pyran-4-carbonitrile (GT-M-159)

4-(((5′-chloro-2′-(piperidin-4-ylamino)-[2,4′-bipyridin]-6-yl)amino)methyl)tetrahydro-2H-pyran-4-carbonitrile (GT-M-159) was prepared by referring to Scheme M-13 (yellow solid, 1.5 g, yield 86.9%). ¹H NMR (400 MHz, DMSO) δ 8.55 (s, 1H), 8.39 (s, 1H), 8.04 (s, 1H), 7.57-7.43 (m, 1H), 7.10 (t, J=6.4 Hz, 1H), 6.95 (d, J=7.3 Hz, 1H), 6.76 (d, J=7.2 Hz, 1H), 6.67 (d, J=10.0 Hz, 2H), 4.08-3.80 (m, 3H), 3.66 (d, J=6.4 Hz, 2H), 3.46 (dd, J=11.9, 10.5 Hz, 2H), 3.29 (s, 2H), 3.04 (dd, J=18.8, 7.9 Hz, 2H), 2.07 (d, J=10.8 Hz, 2H), 1.84 (d, J=13.2 Hz, 2H), 1.73-1.46 (m, 4H). LCMS (ESI) calcd, for C₂₂H₂₈ClN₆O⁺ [M+H]⁺: 427.2; found, 427.2.

### Intermediate Example 25: Preparation of 2-(5-fluoro-2-hydroxyphenyl)-2-(1-oxo-6-(4-(piperazin-1-yl)phenyl)isoindolin-2-yl)-N-(thiazol-2-yl)acetamide (GT-D-012)

2-(5-fluoro-2-hydroxyphenyl)-2-(1-oxo-6-(4-(piperazin-1-yl)phenyl)isoindolin-2-yl)-N-(thiazol-2-yl)acetamide (GT-D-012) was prepared by referring to Scheme M-15 (yellow solid, 110 mg). ¹H NMR (400 MHz, DMSO) § 10.20-8.61 (m, 1H), 7.87 (d, J=8.5 Hz, 2H), 7.63 (dd, J=11.3, 8.4 Hz, 4H), 7.49 (d, J=3.5 Hz, 1H), 7.27 (d, J=3.6 Hz, 1H), 7.12 (td, J=8.6, 3.1 Hz, 1H), 7.07 (d, J=8.8 Hz, 2H), 6.92 (dd, J=8.9, 4.8 Hz, 1H), 6.87 (dd, J=9.2, 3.0 Hz, 1H), 6.33 (s, 1H), 4.64 (d, J=17.5 Hz, 1H), 4.02 (d, J=17.5 Hz, 1H), 3.29 (d, J=4.4 Hz, 5H), 3.12-3.00 (m, 6H), 1.24 (s, 1H). LCMS (ESI) calcd, for C₂₉H₂₇FN₅O₃S⁺ [M+H]⁺: 544.2; found, 544.2.

### Intermediate Example 26: Preparation of 2-(5-fluoro-2-hydroxyphenyl)-2-(1-oxo-6-(6-(piperazin-1-yl)pyridazin-3-yl)isoindolin-2-yl)-N-(thiazol-2-yl)acetamide (GT-D-014)

2-(5-fluoro-2-hydroxyphenyl)-2-(1-oxo-6-(6-(piperazin-1-yl)pyridazin-3-yl)isoindolin-2-yl)-N-(thiazol-2-yl)acetamide (GT-D-014) was prepared by referring to Scheme M-16 (brown solid, 130 mg). ¹H NMR (400 MHz, DMSO) δ 8.31 (d, J=8.3 Hz, 2H), 8.06 (d, J=9.6 Hz, 1H), 7.68 (d, J=7.9 Hz, 1H), 7.48 (d, J=3.6 Hz, 1H), 7.33 (d, J=9.7 Hz, 1H), 7.25 (d, J=3.5 Hz, 1H), 7.11 (td, J=8.6, 3.1 Hz, 1H), 6.94-6.84 (m, 2H), 6.33 (s, 1H), 4.69 (d, J=17.8 Hz, 1H), 4.07 (d, J=17.8 Hz, 1H), 3.66-3.54 (m, 4H), 3.17 (s, 1H), 2.91-2.78 (m, 4H), 1.23 (s, 1H). LCMS (ESI) calcd, for C₂₇H₂₅FN₇O₃S⁺ [M+H]⁺: 546.2; found, 546.2.

### Intermediate Example 27: Preparation of 2-(5-fluoro-2-hydroxyphenyl)-2-(1-oxo-6-(piperazin-1-yl)isoindolin-2-yl)-N-(thiazol-2-yl)acetamide (GT-D-69)

2-(5-fluoro-2-hydroxyphenyl)-2-(1-oxo-6-(piperazin-1-yl)isoindolin-2-yl)-N-(thiazol-2-yl)acetamide (GT-D-69) was prepared by referring to Scheme M-17 (yellow solid, 515 mg). ¹H NMR (400 MHz, DMSO) δ 7.48 (d, J=3.5 Hz, 1H), 7.39 (d, J=8.4 Hz, 1H), 7.24 (dd, J=11.9, 2.8 Hz, 2H), 7.15 (d, J=1.8 Hz, 1H), 7.13-7.05 (m, 1H), 6.90 (dd, J=8.9, 4.8 Hz, 1H), 6.82 (dd, J=9.2, 2.9 Hz, 1H), 6.29 (s, 1H), 4.50 (d, J=17.0 Hz, 1H), 3.89 (d, J=17.0 Hz, 2H), 3.13 (s, 5H), 2.91 (s, 5H), 1.24 (s, 1H). LCMS (ESI) calcd, for C₂₃H₂₃FN₅O₃S⁺ [M+H]⁺: 468.2; found, 468.2.

### Intermediate Example 28: Preparation of 2-(5-fluoro-2-hydroxyphenyl)-2-(6-(4-(methylamino)piperidin-1-yl)-1-oxoisoindolin-2-yl)-N-(thiazol-2-yl)acetamide (GT-D-70)

2-(5-fluoro-2-hydroxyphenyl)-2-(6-(4-(methylamino)piperidin-1-yl)-1-oxoisoindolin-2-yl)-N-(thiazol-2-yl)acetamide (GT-D-70) was prepared by referring to Scheme M-17 (brown solid, 186 mg). ¹H NMR (400 MHz, DMSO) δ 7.40 (dd, J=29.5, 5.8 Hz, 2H), 7.22 (d, J=7.5 Hz, 1H), 7.16 (d, J=19.7 Hz, 3H), 7.05 (d, J=6.2 Hz, 1H), 6.91-6.76 (m, 2H), 6.24 (s, 1H), 4.54 (d, J=17.0 Hz, 1H), 3.94 (d, J=17.0 Hz, 1H), 3.67 (d, J=12.1 Hz, 2H), 3.17 (s, 1H), 2.78 (t, J=11.3 Hz, 2H), 2.31 (d, J=5.8 Hz, 3H), 1.89 (d, J=11.2 Hz, 2H), 1.35 (d, J=11.1 Hz, 2H). LCMS (ESI) calcd, for C₂₅H₂₇FN₅O₃S⁺ [M+H]⁺: 496.2; found, 496.2.

### Intermediate Example 29: Preparation of 2-(5-fluoro-2-hydroxyphenyl)-2-(1-oxo-6-(4-(piperazin-1-yl)piperidin-1-yl)isoindolin-2-yl)-N-(thiazol-2-yl)acetamide (GT-D-71)

2-(5-fluoro-2-hydroxyphenyl)-2-(1-oxo-6-(4-(piperazin-1-yl)piperidin-1-yl)isoindolin-2-yl)-N-(thiazol-2-yl)acetamide (GT-D-71) was prepared by referring to Scheme M-17 (brown solid, 110 mg). ¹H NMR (400 MHz, DMSO) δ 7.45 (s, 1H), 7.36 (d, J=8.4 Hz, 1H), 7.22 (d, J=9.5 Hz, 4H), 7.07 (s, 1H), 6.91-6.77 (m, 3H), 6.25 (s, 1H), 4.51 (d, J=17.1 Hz, 1H), 3.91 (d, J=16.9 Hz, 1H), 3.77 (d, J=12.1 Hz, 3H), 3.17 (s, 2H), 2.72 (s, 5H), 2.44 (s, 5H), 1.82 (d, J=10.7 Hz, 2H), 1.51 (d, J=11.4 Hz, 3H), 1.25 (s, 1H). LCMS (ESI) calcd, for C₂₈H₃₂FN₆O₃S⁺ [M+H]⁺: 551.2; found, 551.2.

### Intermediate Example 30: Preparation of 9-ethyl-6,6-dimethyl-11-oxo-8-(piperazin-1-yl)-6,11-dihydro-5H-benzo[b]carbazole-3-carbonitrile (GT-D-42)

9-ethyl-6,6-dimethyl-11-oxo-8-(piperazin-1-yl)-6,11-dihydro-5H-benzo[b]carbazole-3-carbonitrile (GT-D-42) was prepared by referring to Scheme M-19 (white solid, 240 mg). ¹H NMR (400 MHz, DMSO) δ 13.03 (s, 1H), 9.39 (s, 2H), 8.32 (d, J=8.1 Hz, 1H), 8.05 (d, J=29.2 Hz, 2H), 7.61 (d, J=9.0 Hz, 1H), 7.38 (s, 1H), 3.24 (d, J=14.9 Hz, 8H), 2.73 (d, J=7.5 Hz, 2H), 1.79 (s, 6H), 1.29 (t, J=7.5 Hz, 3H). LCMS (ESI) calcd. for C₂₅H₂₇N₄O⁺ [M+H]⁺: 399.2; found, 399.2.

### Intermediate Example 31: Preparation of 9-ethyl-6,6-dimethyl-11-oxo-8-(4-(piperazin-1-yl)piperidin-1-yl)-6,11-dihydro-5H-benzo[b]carbazole-3-carbonitrile (GT-D-113)

9-ethyl-6,6-dimethyl-11-oxo-8-(4-(piperazin-1-yl)piperidin-1-yl)-6,11-dihydro-5H-benzo[b]carbazole-3-carbonitrile (GT-D-113) was prepared by referring to Scheme M-19 (off-white solid, 1.4 g). ¹H NMR (400 MHz, MeOD) δ 8.39 (d, J=8.2 Hz, 1H), 8.23 (s, 1H), 7.86 (s, 1H), 7.62-7.48 (m, 2H), 3.75 (s, 9H), 3.53 (d, J=12.0 Hz, 2H), 3.18 (t, J=11.7 Hz, 2H), 2.86 (q, J=7.4 Hz, 2H), 2.43 (d, J=11.4 Hz, 2H), 2.22 (dd, J=20.7, 11.9 Hz, 2H), 1.82 (s, 6H), 1.37 (t, J=7.5 Hz, 3H). LCMS (ESI) calcd, for C₃₀H₃₆N₅O⁺ [M+H]⁺: 482.3; found, 482.2.

### Intermediate Example 32: Preparation of 9-ethyl-6,6-dimethyl-8-(4-(methylamino)piperidin-1-yl)-11-oxo-6,11-dihydro-5H-benzo[b]carbazole-3-carbonitrile (GT-D-85)

9-ethyl-6,6-dimethyl-8-(4-(methylamino)piperidin-1-yl)-11-oxo-6,11-dihydro-5H-benzo[b]carbazole-3-carbonitrile (GT-D-85) was prepared by referring to Scheme M-19 (white solid, 130 mg). ¹H NMR (400 MHz, MeOD) δ 8.31 (d, J=8.2 Hz, 1H), 8.11 (s, 1H), 7.77 (s, 1H), 7.45 (d, J=8.2 Hz, 1H), 7.36 (s, 1H), 3.29 (d, J=11.9 Hz, 2H), 2.91 (t, J=11.7 Hz, 2H), 2.78-2.64 (m, 5H), 2.18 (d, J=11.7 Hz, 2H), 1.86-1.75 (m, 2H), 1.71 (s, 6H), 1.25 (t, J=7.5 Hz, 3H), 1.22-1.18 (m, 1H). LCMS (ESI) calcd, for C₂₇H₃₁N₄O⁺ [M+H]⁺: 427.3; found, 427.2.

### Intermediate Example 33: Preparation of 9-cyclopentyl-N₈-phenyl-N₂-(4-(piperazin-1-yl)phenyl)-9H-purine-2,8-diamine (GT-D-62)

9-cyclopentyl-N₈-phenyl-N₂-(4-(piperazin-1-yl)phenyl)-9H-purine-2,8-diamine (GT-D-62) was prepared by referring to Scheme M-20 (white solid, 1.18 g). ¹H NMR (400 MHz, MeOD) δ 8.15 (s, 1H), 7.67-7.52 (m, 4H), 7.46 (dd, J=15.9, 8.0 Hz, 3H), 7.25 (d, J=9.0 Hz, 2H), 3.66 (s, 1H), 3.61-3.53 (m, 4H), 3.47 (dd, J=6.5, 3.6 Hz, 4H), 2.40 (dd, J=12.8, 7.3 Hz, 2H), 2.20 (d, J=7.9 Hz, 2H), 1.92 (s, 2H), 1.77-1.62 (m, 2H). LCMS (ESI) calcd, for C₂₆H₃₁N₈⁺ [M+H]⁺: 455.3; found, 455.0.

### Intermediate Example 34: Preparation of 9-cyclopentyl-N₂-(4-(4-(methylamino)piperidin-1-yl)phenyl)-N₈-phenyl-9H-purine-2,8-diamine (GT-D-67)

9-cyclopentyl-N₂-(4-(4-(methylamino)piperidin-1-yl)phenyl)-N₈-phenyl-9H-purine-2,8-diamine (GT-D-67) was prepared by referring to Scheme M-20 (yellow solid, 4.6 g). ¹H NMR (400 MHz, MeOD) δ 8.34 (s, 1H), 7.94-7.82 (m, 4H), 7.57 (dd, J=7.4, 6.3 Hz, 4H), 7.45 (dd, J=7.7, 4.2 Hz, 1H), 3.95-3.81 (m, 4H), 3.71-3.60 (m, 2H), 2.89 (s, 1H), 2.81 (s, 3H), 2.51 (t, J=15.4 Hz, 4H), 2.43-2.37 (m, 2H), 2.25 (d, J=8.3 Hz, 2H), 2.09-2.02 (m, 2H), 1.83-1.71 (m, 2H). LCMS (ESI) calcd, for C₂₈H₃₅N₈⁺ [M+H]⁺: 483.3; found, 483.3.

### Intermediate Example 35: Preparation of 9-cyclopentyl-N₈-phenyl-N₂-(4-(4-(piperazin-1-yl)piperidin-1-yl)phenyl)-9H-purine-2,8-diamine (GT-D-68)

9-cyclopentyl-N₈-phenyl-N₂-(4-(4-(piperazin-1-yl)piperidin-1-yl)phenyl)-9H-purine-2,8-diamine (GT-D-68) was prepared by referring to Scheme M-20 (yellow solid, 4.1 g). ¹H NMR (400 MHz, DMSO) δ 7.62 (d, J=8.9 Hz, 2H), 7.56 (t, J=3.7 Hz, 3H), 7.01-6.94 (m, 2H), 6.80 (d, J=9.0 Hz, 2H), 6.42 (t, J=7.1 Hz, 1H), 3.53 (d, J=11.9 Hz, 2H), 2.67 (s, 4H), 2.54 (d, J=10.1 Hz, 2H), 2.42 (s, 2H), 2.38-2.31 (m, 6H), 2.20 (t, J=11.2 Hz, 1H), 1.99 (s, 2H), 1.92-1.87 (m, 1H), 1.81 (d, J=10.0 Hz, 4H), 1.69-1.61 (m, 2H), 1.52 (dt, J=11.7, 8.7 Hz, 2H). LCMS (ESI) calcd, for C₃₁H₄₀N₉⁺ [M+H]⁺: 538.3; found, 538.4.

### Intermediate Example 36: Preparation of N-(2-(1-(cyclopropylsulfonyl)-1H-pyrazol-4-yl)pyrimidin-4-yl)-1-isopropyl-3-(piperazin-1-yl)-1H-pyrazolo[4,3-c]pyridin-6-amine (GT-D-18)

N-(2-(1-(cyclopropylsulfonyl)-1H-pyrazol-4-yl)pyrimidin-4-yl)-1-isopropyl-3-(piperazin-1-yl)-1H-pyrazolo[4,3-c]pyridin-6-amine (GT-D-18) was prepared by referring to Scheme M-18 (yellow solid, 0.75 g). ¹H NMR (400 MHz, CDCl₃) δ 8.73 (s, 1H), 8.69 (s, 1H), 8.47 (s, 1H), 8.39 (d, J=5.9 Hz, 1H), 8.12 (s, 1H), 7.97 (s, 1H), 6.81 (d, J=5.8 Hz, 1H), 4.65 (dt, J=13.3, 6.6 Hz, 1H), 3.56-3.43 (m, 4H), 3.16-3.00 (m, 4H), 2.90-2.80 (m, 1H), 1.58 (d, J=6.7 Hz, 6H), 1.55-1.49 (m, 2H), 1.26-1.19 (m, 2H). LCMS (ESI) calcd. for C₂₃H₂₉N₁₀O₂S⁺ [M+H]⁺: 509.2; found, 509.2.

### Intermediate Example 37: Preparation of N-(2-(1-(cyclopropylsulfonyl)-1H-pyrazol-4-yl)pyrimidin-4-yl)-1-isopropyl-3-(4-(methylamino)piperidin-1-yl)-1H-pyrazolo[4,3-c]pyridin-6-amine (GT-D-72)

N-(2-(1-(cyclopropylsulfonyl)-1H-pyrazol-4-yl)pyrimidin-4-yl)-1-isopropyl-3-(4-(methylamino)piperidin-1-yl)-1H-pyrazolo[4,3-c]pyridin-6-amine (GT-D-72) was prepared by referring to Scheme M-18 (yellow solid, 0.4 g). ¹H NMR (400 MHz, CDCl₃) δ 8.73 (s, 1H), 8.69 (s, 1H), 8.47 (s, 1H), 8.40 (d, J=5.9 Hz, 1H), 8.11 (s, 1H), 7.80 (s, 1H), 6.82 (d, J=5.8 Hz, 1H), 4.65 (dt, J=13.3, 6.6 Hz, 1H), 4.09 (t, J=12.3 Hz, 2H), 3.05 (t, J=11.3 Hz, 2H), 2.85 (ddd, J=12.5, 7.8, 4.7 Hz, 2H), 2.61 (s, 3H), 2.16 (d, J=10.7 Hz, 2H), 1.81 (dd, J=20.3, 11.2 Hz, 2H), 1.58 (d, J=6.7 Hz, 6H), 1.54 (dd, J=5.8, 3.7 Hz, 2H), 1.23 (dt, J=7.0, 3.6 Hz, 2H). LCMS (ESI) calcd, for C₂₅H₃₃N₁₀O₂S⁺ [M+H]⁺: 537.3; found, 537.4.

### Intermediate Example 38: Preparation of N-(2-(1-(cyclopropylsulfonyl)-1H-pyrazol-4-yl)pyrimidin-4-yl)-1-isopropyl-3-(4-(piperazin-1-yl)piperidin-1-yl)-1H-pyrazolo[4,3-c]pyridin-6-amine (GT-D-73)

N-(2-(1-(cyclopropylsulfonyl)-1H-pyrazol-4-yl)pyrimidin-4-yl)-1-isopropyl-3-(4-(piperazin-1-yl)piperidin-1-yl)-1H-pyrazolo[4,3-c]pyridin-6-amine (GT-D-73) was prepared by referring to Scheme M-18 (yellow solid, 1.19 g). ¹H NMR (400 MHz, DMSO) δ 10.37 (s, 1H), 8.86 (s, 1H), 8.70 (s, 1H), 8.48 (s, 1H), 8.41 (d, J=5.9 Hz, 1H), 8.32 (s, 1H), 7.13 (d, J=5.5 Hz, 1H), 4.72 (s, 1H), 4.02 (d, J=12.0 Hz, 2H), 3.53 (d, J=57.7 Hz, 1H), 3.33-3.27 (m, 2H), 2.88 (t, J=11.6 Hz, 2H), 2.76 (s, 4H), 2.35 (d, J=12.0 Hz, 1H), 1.87 (d, J=10.4 Hz, 2H), 1.60 (d, J=10.0 Hz, 2H), 1.47 (d, J=6.6 Hz, 6H), 1.36 (s, 2H), 1.28 (dd, J=7.5, 5.1 Hz, 2H). LCMS (ESI) calcd, for C₂₈H₃₈N₁₁O₂S⁺ [M+H]⁺: 592.3; found, 592.4.

### Intermediate Example 39: Preparation of 1-isopropyl-N-(2-(4-methoxypiperidin-1-yl)pyrimidin-4-yl)-3-(4-(methylamino)piperidin-1-yl)-1H-pyrazolo[4,3-c]pyridin-6-amine (GT-D-74)

1-isopropyl-N-(2-(4-methoxypiperidin-1-yl)pyrimidin-4-yl)-3-(4-(methylamino)piperidin-1-yl)-1H-pyrazolo[4,3-c]pyridin-6-amine (GT-D-74) was prepared by referring to Scheme M-18 (yellow solid, 0.97 g). ¹H NMR (400 MHz, MeOD) δ 9.38 (s, 1H), 8.03 (d, J=7.1 Hz, 1H), 7.80 (s, 1H), 6.59 (d, J=7.1 Hz, 1H), 4.89 (s, 1H), 4.30 (d, J=13.0 Hz, 2H), 3.89 (d, J=8.7 Hz, 2H), 3.67 (d, J=15.3 Hz, 2H), 3.59 (s, 1H), 3.39 (s, 3H), 3.37-3.32 (m, 1H), 3.20 (s, 2H), 2.77 (s, 3H), 2.27 (d, J=11.6 Hz, 2H), 2.07-1.94 (m, 2H), 1.83 (ddd, J=33.0, 16.1, 8.8 Hz, 4H), 1.54 (d, J=6.5 Hz, 6H). LCMS (ESI) calcd, for C₂₅H₃₈N₉O⁺ [M+H]⁺: 480.3; found, 480.4.

### Intermediate Example 40: Preparation of 1-isopropyl-N-(2-(4-methoxypiperidin-1-yl)pyrimidin-4-yl)-3-(4-(piperazin-1-yl)piperidin-1-yl)-1H-pyrazolo[4,3-c]pyridin-6-amine (GT-D-75)

1-isopropyl-N-(2-(4-methoxypiperidin-1-yl)pyrimidin-4-yl)-3-(4-(piperazin-1-yl)piperidin-1-yl)-1H-pyrazolo[4,3-c]pyridin-6-amine (GT-D-75) was prepared by referring to Scheme M-18 (yellow solid, 1.1 g). ¹H NMR (400 MHz, MeOD) δ 9.40 (s, 1H), 8.05 (d, J=7.0 Hz, 1H), 7.83 (s, 1H), 6.61 (d, J=7.0 Hz, 1H), 4.39 (d, J=12.7 Hz, 2H), 3.93 (s, 3H), 3.67 (dd, J=33.6, 15.5 Hz, 12H), 3.41 (s, 3H), 3.23 (t, J=12.4 Hz, 2H), 2.41 (d, J=10.4 Hz, 2H), 2.07 (dd, J=23.8, 8.9 Hz, 4H), 1.79 (s, 2H), 1.57 (d, J=6.4 Hz, 6H). LCMS (ESI) calcd. for C₂₈H₄₃N₁₀O⁺ [M+H]⁺: 535.4; found, 535.5.

### Intermediate Example 41: Preparation of 7-cyclopentyl-N,N-dimethyl-2-((5-(4-(methylamino)piperidin-1-yl)pyridin-2-yl)amino)-7H-pyrrolo[2,3-d]pyrimidine-6-carboxamide (GT-D-79)

7-cyclopentyl-N,N-dimethyl-2-((5-(4-(methylamino)piperidin-1-yl)pyridin-2-yl)amino)-7H-pyrrolo[2,3-d]pyrimidine-6-carboxamide (GT-D-79) was prepared by referring to Scheme M-8 (yellow solid, 800 mg). ¹H NMR (400 MHz, DMSO) δ 9.38 (s, 1H), 8.77 (s, 1H), 8.15 (d, J=9.1 Hz, 1H), 8.03 (d, J=2.9 Hz, 1H), 7.45 (dd, J=9.1, 3.0 Hz, 1H), 6.60 (s, 1H), 4.74 (p, J=8.8 Hz, 1H), 3.67 (d, J=12.5 Hz, 2H), 3.51 (s, 1H), 3.06 (s, 6H), 2.90-2.80 (m, 1H), 2.72 (t, J=11.1 Hz, 2H), 2.47 (s, 3H), 2.43 (d, J=11.5 Hz, 2H), 2.05-1.94 (m, 6H), 1.67-1.51 (m, 4H). LCMS (ESI) calcd, for C₂₅H₃₅N₈O⁺ [M+H]⁺: 463.3; found, 463.2.

### Intermediate Example 42: Preparation of 7-cyclopentyl-N,N-dimethyl-2-((5-(4-(piperazin-1-yl)piperidin-1-yl)pyridin-2-yl)amino)-7H-pyrrolo[2,3-d]pyrimidine-6-carboxamide (GT-D-80)

7-cyclopentyl-N,N-dimethyl-2-((5-(4-(piperazin-1-yl)piperidin-1-yl)pyridin-2-yl)amino)-7H-pyrrolo[2,3-d]pyrimidine-6-carboxamide (GT-D-80) was prepared by referring to Scheme M-8 (off-white solid, 305 mg). ¹H NMR (400 MHz, DMSO) δ 9.22 (s, 1H), 8.74 (s, 1H), 8.12 (d, J=9.2 Hz, 1H), 7.98 (d, J=2.8 Hz, 1H), 7.42 (dd, J=9.1, 2.9 Hz, 1H), 6.59 (s, 1H), 4.80-4.66 (m, 1H), 3.05 (s, 6H), 2.70-2.66 (m, 4H), 2.63 (d, J=11.8 Hz, 2H), 2.42 (s, 6H), 2.24 (d, J=11.4 Hz, 1H), 1.97 (s, 6H), 1.84 (d, J=11.2 Hz, 2H), 1.64 (s, 2H), 1.58-1.50 (m, 2H), 1.23 (s, 1H). LCMS (ESI) calcd, for C₂₈H₄₀N₉O⁺ [M+H]⁺: 518.3; found, 518.4.

### Intermediate Example 43: Preparation of N-(3-(difluoromethyl)-1-(piperidin-4-yl)-1H-pyrazol-4-yl)-5-morpholinopyrazolo[1,5-a]pyrimidine-3-carboxamide hydrochloride (GT-D-101)

N-(3-(difluoromethyl)-1-(piperidin-4-yl)-1H-pyrazol-4-yl)-5-morpholinopyrazolo[1,5-a]pyrimidine-3-carboxamide hydrochloride (GT-D-101) was prepared by referring to Scheme M-35 (yellow solid, 1.3 g). ¹H NMR (400 MHz, MeOD) δ 8.55 (d, J=8.0 Hz, 1H), 8.42 (s, 1H), 8.32 (s, 1H), 7.03-6.75 (m, 2H), 4.60 (s, 1H), 3.86-3.80 (m, 8H), 3.58 (dd, J=10.0, 6.5 Hz, 2H), 3.27-3.17 (m, 2H), 2.39-2.24 (m, 4H). LCMS (ESI) calcd. for C₂₀H₂₅F₂N₈O₂⁺ [M+H]⁺: 447.2; found, 447.2.

### Intermediate Example 44: Preparation of 3-(imidazo[1,2-b]pyridazin-3-ylethynyl)-4-methyl-N-(4-(piperazin-1-ylmethyl)-3-(trifluoromethyl)phenyl)benzamide hydrochloride (GT-D-109)

3-(imidazo[1,2-b]pyridazin-3-ylethynyl)-4-methyl-N-(4-(piperazin-1-ylmethyl)-3-(trifluoromethyl)phenyl)benzamide hydrochloride (GT-D-109) was prepared by referring to Scheme M-22 (off-white solid, 670 mg). ¹H NMR (400 MHz, MeOD) δ 9.10 (dd, J=4.5, 1.3 Hz, 1H), 8.62 (s, 1H), 8.52 (dd, J=9.3, 1.3 Hz, 1H), 8.39 (d, J=1.8 Hz, 1H), 8.24 (d, J=1.8 Hz, 1H), 8.14 (d, J=1.8 Hz, 1H), 8.09 (d, J=8.6 Hz, 1H), 7.98 (ddd, J=13.9, 8.7, 3.2 Hz, 2H), 7.55 (d, J=8.1 Hz, 1H), 4.53 (s, 2H), 3.60 (dd, J=26.0, 12.5 Hz, 8H), 2.69 (s, 3H). LCMS (ESI) calcd, for C₂₈H₂₆F₃N₆₀⁺ [M+H]⁺: 519.2; found, 519.4.

### Intermediate Example 45: Preparation of 3-(imidazo[1,2-b]pyridazin-3-ylethynyl)-4-methyl-N-(4-(piperazin-1-yl)-3-(trifluoromethyl)phenyl)benzamide (GT-D-110)

3-(imidazo[1,2-b]pyridazin-3-ylethynyl)-4-methyl-N-(4-(piperazin-1-yl)-3-(trifluoromethyl)phenyl)benzamide (GT-D-110) was prepared by referring to Scheme M-21 (off-white solid, 930 mg). ¹H NMR (400 MHz, MeOD) δ 9.10-9.00 (m, 1H), 8.55 (s, 1H), 8.47 (dd, J=9.3, 1.3 Hz, 1H), 8.22 (d, J=1.8 Hz, 1H), 8.15 (d, J=2.4 Hz, 1H), 8.05 (dd, J=8.7, 2.4 Hz, 1H), 7.97 (dd, J=8.0, 1.9 Hz, 1H), 7.89 (dd, J=9.3, 4.5 Hz, 1H), 7.65 (t, J=6.5 Hz, 1H), 7.59-7.56 (m, 1H), 3.38-3.34 (m, 4H), 3.23-3.14 (m, 4H), 2.68 (s, 3H). LCMS (ESI) calcd, for C₂₇H₂₄F₃N₆₀⁺ [M+H]⁺: 505.2; found, 505.3.

### Intermediate Example 46: Preparation of 3-(imidazo[1,2-b]pyridazin-3-ylethynyl)-4-methyl-N-(4-(4-(methylamino)piperidin-1-yl)-3-(trifluoromethyl)phenyl)benzamide (GT-D-111)

3-(imidazo[1,2-b]pyridazin-3-ylethynyl)-4-methyl-N-(4-(4-(methylamino)piperidin-1-yl)-3-(trifluoromethyl)phenyl)benzamide (GT-D-111) was prepared by referring to Scheme M-21 (white solid, 1.8 g). ¹H NMR (400 MHz, DMSO) δ 10.63 (s, 1H), 9.21 (s, 2H), 8.86 (dd, J=4.4, 1.1 Hz, 1H), 8.43 (s, 1H), 8.21 (dd, J=14.6, 1.9 Hz, 2H), 8.08 (s, 1H), 8.00 (dd, J=8.0, 1.6 Hz, 1H), 7.59-7.53 (m, 3H), 3.00 (d, J=11.7 Hz, 3H), 2.78 (s, 2H), 2.61 (s, 3H), 2.54 (t, J=5.4 Hz, 3H), 2.11 (d, J=10.0 Hz, 2H), 1.70 (dt, J=11.3, 8.1 Hz, 2H). LCMS (ESI) calcd, for C₂₉H₂₈F₃N₆O⁺ [M+H]⁺: 533.2; found, 533.2.

### Intermediate Example 47: Preparation of 3-(imidazo[1,2-b]pyridazin-3-ylethynyl)-4-methyl-N-(4-(4-(piperidin-4-yl)piperazin-1-yl)-3-(trifluoromethyl)phenyl)benzamide (GT-D-112)

3-(imidazo[1,2-b]pyridazin-3-ylethynyl)-4-methyl-N-(4-(4-(piperidin-4-yl)piperazin-1-yl)-3-(trifluoromethyl)phenyl)benzamide (GT-D-112) was prepared by referring to Scheme M-21 (brown solid, 2.1 g). ¹H NMR (400 MHz, DMSO) & 10.60 (s, 1H), 8.74 (dd, J=4.4, 1.5 Hz, 1H), 8.28 (dd, J=9.2, 1.5 Hz, 1H), 8.25 (s, 1H), 8.22 (d, J=2.1 Hz, 2H), 8.19-8.13 (m, 1H), 7.96 (dd, J=8.0, 1.8 Hz, 1H), 7.58 (t, J=9.0 Hz, 2H), 7.42 (dd, J=9.2, 4.5 Hz, 1H), 3.49 (d, J=13.0 Hz, 4H), 3.15 (s, 6H), 2.98-2.82 (m, 2H), 2.62 (s, 3H), 2.33 (d, J=10.9 Hz, 2H), 1.80 (d, J=9.7 Hz, 2H). LCMS (ESI) calcd, for C₃₂H₃₃F₃N₂O⁺ [M+H]⁺: 588.3; found, 588.2.

### Intermediate Example 48: Preparation of tert-butyl (4-(4-((3-(difluoromethyl)-1-(piperidin-4-yl)-1H-pyrazol-4-yl) carbamoyl) oxazol-2-yl)pyridin-2-yl) (2,2,2-trifluoroethyl) carbamate (GT-D-126)

tert-butyl (4-(4-((3-(difluoromethyl)-1-(piperidin-4-yl)-1H-pyrazol-4-yl) carbamoyl) oxazol-2-yl)pyridin-2-yl) (2,2,2-trifluoroethyl) carbamate (GT-D-126) was prepared by referring to Scheme M-23 (white solid, 2.2 g). ¹H NMR (400 MHz, CDCl₃) δ 9.06 (s, 1H), 8.53 (d, J=5.1 Hz, 1H), 8.39 (s, 1H), 8.34 (d, J=5.3 Hz, 2H), 7.72 (dd, J=5.1, 1.2 Hz, 1H), 6.84 (t, J=54.7 Hz, 1H), 4.88 (q, J=8.7 Hz, 2H), 4.21 (s, 1H), 3.26 (d, J=12.6 Hz, 2H), 2.77 (td, J=12.5, 2.1 Hz, 2H), 2.16 (d, J=12.2 Hz, 2H), 1.92 (dd, J=12.1, 3.8 Hz, 2H), 1.58 (s, 9H). LCMS (ESI) calcd, for C₂₅H₂₉F₅N₇O₄⁺ [M+H]⁺: 586.2; found, 586.3.

### Intermediate Example 49: Preparation of (1r,4r)-4-(6-(5-cyano-1H-pyrrolo[2,3-b]pyridin-1-yl)-4-(isopropylamino) nicotinamido)cyclohexane-1-carboxylic acid (GT-D-132)

(1r,4r)-4-(6-(5-cyano-1H-pyrrolo[2,3-b]pyridin-1-yl)-4-(isopropylamino) nicotinamido)cyclohexane-1-carboxylic acid (GT-D-132) was prepared by referring to Scheme M-25 (white solid, 3.1 g). ¹H NMR (400 MHz, DMSO) δ 12.11 (s, 1H), 8.81 (d, J=1.9 Hz, 1H), 8.67 (d, J=2.0 Hz, 1H), 8.63-8.55 (m, 2H), 8.52 (d, J=3.9 Hz, 1H), 8.37 (d, J=7.6 Hz, 1H), 8.07 (s, 1H), 6.89 (d, J=3.9 Hz, 1H), 3.77 (dt, J=19.1, 6.3 Hz, 2H), 2.17 (dd, J=11.2, 7.9 Hz, 1H), 2.04-1.85 (m, 4H), 1.39 (dd, J=16.9, 8.0 Hz, 4H), 1.30 (s, 3H), 1.29 (s, 3H). LCMS (ESI) calcd, for C₂₄H₂₇N₆O₃⁺ [M+H]⁺: 447.2; found, 447.2.

### Intermediate Example 50: Preparation of 6-(5-cyano-1H-pyrrolo[2,3-b]pyridin-1-yl)-4-(isopropylamino)-N-(piperidin-4-yl) nicotinamide (GT-D-133)

6-(5-cyano-1H-pyrrolo[2,3-b]pyridin-1-yl)-4-(isopropylamino)-N-(piperidin-4-yl) nicotinamide (GT-D-133) was prepared by referring to Scheme M-24 (white solid, 2.8 g). ¹H NMR (400 MHz, MeOD) δ 8.80 (d, J=1.3 Hz, 1H), 8.74 (s, 1H), 8.62 (d, J=1.5 Hz, 1H), 8.39 (d, J=4.0 Hz, 1H), 7.44 (s, 1H), 7.09 (d, J=4.0 Hz, 1H), 4.34-4.04 (m, 2H), 3.52 (d, J=13.1 Hz, 2H), 3.17 (dd, J=12.4, 10.3 Hz, 2H), 2.23 (d, J=11.3 Hz, 2H), 1.97 (td, J=14.5, 4.2 Hz, 2H), 1.39 (d, J=6.4 Hz, 6H). LCMS (ESI) calcd, for C₂₂H₂₆N₇O⁺ [M+H]⁺: 404.2; found, 404.3.

### Intermediate Example 51: Preparation of N-(4-(chlorodifluoromethoxy)phenyl)-6-(4-(piperazin-1-yl)piperidin-1-yl)-5-(1H-pyrazol-5-yl) nicotinamide (GT-D-179)

N-(4-(chlorodifluoromethoxy)phenyl)-6-(4-(piperazin-1-yl)piperidin-1-yl)-5-(1H-pyrazol-5-yl) nicotinamide (GT-D-179) was prepared by referring to Scheme M-26 (brown solid, 1.03 g). ¹H NMR (400 MHz, MeOD) δ 8.79 (d, J=2.3 Hz, 1H), 8.68 (d, J=2.3 Hz, 1H), 8.00 (d, J=2.3 Hz, 1H), 7.88-7.80 (m, 2H), 7.30 (d, J=9.1 Hz, 2H), 6.88 (d, J=2.2 Hz, 1H), 3.90 (dd, J=11.4, 5.8 Hz, 3H), 3.72 (s, 8H), 3.24 (d, J=12.2 Hz, 2H), 2.31 (d, J=10.7 Hz, 2H), 2.09 (dd, J=16.3, 7.7 Hz, 2H). LCMS (ESI) calcd, for C₂₅H₂₉ClF₂N₇O₂⁺ [M+H]⁺: 532.2; found, 532.2.

### Intermediate Example 52: Preparation of (6-((5-bromo-2-((2-methoxy-5-methyl-4-(piperazin-1-yl)phenyl)amino)pyrimidin-4-yl)amino)quinoxalin-5-yl)dimethylphosphine oxide (GT-S-50)

(6-((5-bromo-2-((2-methoxy-5-methyl-4-(piperazin-1-yl)phenyl)amino)pyrimidin-4-yl)amino)quinoxalin-5-yl)dimethylphosphine oxide (GT-S-50) was prepared by referring to Scheme M-8 (yellow solid, 950 mg). ¹H NMR (400 MHz, DMSO-d₆): δ ppm 12.76 (s, 1H), 9.23 (br, 2H), 8.91 (d, 3H), 8.38-8.32 (m, 2H), 8.00 (d, 1H), 7.49 (s, 1H), 6.77 (s, 1H), 3.83 (s, 3H), 3.32-3.24 (m, 4H), 3.12-2.97 (m, 4H), 2.13 (s, 3H), 2.10 (s, 3H), 2.03 (s, 3H). LCMS (ESI) calcd, for C₂₆H₃₁BrN₈O₂P⁺ [M+H]⁺: 597.2; found, 597.1.

### Intermediate Example 53: Preparation of (6-((5-bromo-2-((2-methoxy-5-methyl-4-(4-(methylamino)piperidin-1-yl)phenyl)amino)pyrimidin-4-yl)amino)quinoxalin-5-yl)dimethylphosphine oxide (GT-S-51)

(6-((5-bromo-2-((2-methoxy-5-methyl-4-(4-(methylamino)piperidin-1-yl)phenyl)amino)pyrimidin-4-yl)amino)quinoxalin-5-yl)dimethylphosphine oxide (GT-S-51) was prepared by referring to Scheme M-8 (yellow solid, 1.1 g). ¹H NMR (400 MHz, CD₃OD): δ ppm 8.94-8.90 (br, 3H), 8.30 (s, 1H), 8.10-8.07 (br, 1H), 7.35 (s, 1H), 6.89 (s, 1H), 3.91 (s, 3H), 3.39-3.24 (m, 3H), 2.91 (t, J=15.6 Hz, 2H), 2.82 (s, 3H), 2.30-2.19 (m, 11H), 1.98-1.84 (m, 11H), 1.98-1.84 (m, 2H). LCMS (ESI) calcd, for C₂₈H₃₅BrN₈O₂P⁺ [M+H]⁺: 625.2; found, 625.1.

### Intermediate Example 54: Preparation of (6-((5-bromo-2-((2-methoxy-5-methyl-4-(4-(piperazin-1-yl)piperidin-1-yl)phenyl)amino)pyrimidin-4-yl)amino)quinoxalin-5-yl)dimethylphosphine oxide (GT-S-52)

(6-((5-bromo-2-((2-methoxy-5-methyl-4-(4-(piperazin-1-yl)piperidin-1-yl)phenyl)amino)pyrimidin-4-yl)amino)quinoxalin-5-yl)dimethylphosphine oxide (GT-S-52) was prepared by referring to Scheme M-8 (yellow solid, 150 mg, yield 108.88%). ¹H NMR (400 MHz, DMSO-d₆) δ ppm 9.10-8.80 (m, 2H), 8.41 (s, 1H), 8.05-7.95 (m, 1H), 7.60-7.50 (m, 2H), 7.48-7.36 (m, 3H), 7.20-7.10 (m, 2H), 6.85-6.80 (m, 1H), 3.80-3.62 (m, 6H), 3.60-3.40 (m, 6H), 3.35-3.30 (m, 2H), 2.85-2.60 (m, 2H), 2.38-2.25 (m, 2H), 2.20-1.90 (m, 11H). LCMS (ESI) calcd, for C₃₁H₄₀BrN₉O₂P⁺ [M+H]⁺: 680.2; found, 680.1.

### Intermediate Example 55: Preparation of 1-isopropyl-N-((6-methyl-2-oxo-4-propyl-1,2-dihydropyridin-3-yl)methyl)-6-(6-(piperazin-1-yl)pyridin-3-yl)-1H-indazole-4-carboxamide (GT-S-7)

1-isopropyl-N-((6-methyl-2-oxo-4-propyl-1,2-dihydropyridin-3-yl)methyl)-6-(6-(piperazin-1-yl)pyridin-3-yl)-1H-indazole-4-carboxamide (GT-S-7) was prepared by referring to Scheme M-29 (yellow solid, 300 mg, yield 21%). ¹H NMR (400 MHz, DMSO-d₆) δ ppm 8.67-8.61 (m, 2H), 8.38 (s, 1H), 8.08-8.05 (br, 2H), 7.85 (s, 1H), 6.96 (d, J=11.6 Hz, 1H), 5.94 (s, 1H), 5.20-5.11 (m, 1H), 4.44 (d, J=6.4 Hz, 2H), 3.52-3.49 (m, 4H), 2.86-2.78 (m, 4H), 2.57-2.53 (m, 2H), 2.11 (s, 3H), 1.58-1.51 (m, 8H), 0.90 (t, J=9.6 Hz, 3 H). LCMS (ESI) calcd, for C₃₀H₃₈N₇O₂⁺ [M+H]⁺: 528.3; found, 528.5.

### Intermediate Example 56: Preparation of 1-isopropyl-N-((6-methyl-2-oxo-4-propyl-1,2-dihydropyridin-3-yl)methyl)-6-(6-(4-(methylamino)piperidin-1-yl)pyridin-3-yl)-1H-indazole-4-carboxamide (GT-S-8)

1-isopropyl-N-((6-methyl-2-oxo-4-propyl-1,2-dihydropyridin-3-yl)methyl)-6-(6-(4-(methylamino)piperidin-1-yl)pyridin-3-yl)-1H-indazole-4-carboxamide (GT-S-8) was prepared by referring to Scheme M-27 (light yellow solid, 1.8 g). ¹H NMR (400 MHz, DMSO-d₆): δ ppm 8.69-8.57 (m, 4H), 8.63 (s, 1H), 8.15-8.08 (m, 4H), 7.82 (br, 1H), 7.11 (d, J=8.8 Hz, 1H), 5.91 (s, 1H), 5.17-5.10 (m, 1H), 4.48-4.41 (m, 4H), 3.30-3.28 (br, 1H), 2.96 (t, J=12.6 Hz, 2H), 2.59 (t, J=5.2 Hz, 3H), 2.54-2.49 (m, 4H), 2.14 (s, 3H), 2.09-2.07 (m, 2H), 1.57-1.49 (m, 8H), 0.88 (t, J=7.4 Hz, 3H). LCMS (ESI) calcd, for C₃₂H₄₂N₇O₂⁺ [M+H]⁺: 556.3; found, 556.5.

### Intermediate Example 57: Preparation of 1-isopropyl-N-((6-methyl-2-oxo-4-propyl-1,2-dihydropyridin-3-yl)methyl)-6-(6-(4-(piperidin-4-yl)piperazin-1-yl)pyridin-3-yl)-1H-indazole-4-carboxamide (GT-S-9)

1-isopropyl-N-((6-methyl-2-oxo-4-propyl-1,2-dihydropyridin-3-yl)methyl)-6-(6-(4-(piperidin-4-yl)piperazin-1-yl)pyridin-3-yl)-1H-indazole-4-carboxamide (GT-S-9) was prepared by referring to Scheme M-28 (light yellow solid, 2.0 g). ¹H NMR (400 MHz, DMSO-d₆): δ ppm 11.99 (br, 1H), 9.40-9.37 (br, 1H), 8.78 (br, 1H), 8.67 (s, 1H), 8.45-8.40 (m, 2H), 8.24 (s, 1H), 7.88 (s, 1H), 7.40 (d, J=12 Hz, 1H), 5.99 (s, 1H), 5.24-5.15 (m, 1H), 4.66-4.62 (m, 2H), 4.45-4.44 (br, 2H), 3.77-3.18 (m, 7H), 3.25-3.18 (m, 2H), 2.97-2.90 (m, 2H), 2.58-2.52 (m, 4H), 2.37-2.33 (br, 2H), 2.17 (s, 3H), 2.10-2.02 (m, 2H), 1.57-1.51 (m, 8H), 0.89 (t, J=9.6 Hz, 3H). LCMS (ESI) calcd, for C₃₅H₄₇N₈O₂⁺ [M+H]⁺: 611.4; found, 611.7.

### Intermediate Example 58: Preparation of N-((4,6-dimethyl-2-oxo-1,2-dihydropyridin-3-yl)methyl)-5-(ethyl(tetrahydro-2H-pyran-4-yl)amino)-4-methyl-4′-(piperazin-1-ylmethyl)-[1,1′-biphenyl]-3-carboxamide (GT-S-10)

N-((4,6-dimethyl-2-oxo-1,2-dihydropyridin-3-yl)methyl)-5-(ethyl(tetrahydro-2H-pyran-4-yl)amino)-4-methyl-4′-(piperazin-1-ylmethyl)-[1,1′-biphenyl]-3-carboxamide (GT-S-10) was prepared by referring to Scheme M-31 (yellow solid, 1.4 g). ¹H NMR (400 MHz, DMSO-d₆): δ ppm 11.99 (br, 1H), 9.40-9.37 (br, 1H), 8.78 (br, 1H), 8.67 (s, 1H), 8.45-8.40 (m, 2H), 8.24 (s, 1H), 7.88 (s, 1H), 7.40 (d, J=12 Hz, 1H), 5.99 (s, 1H), 5.24-5.15 (m, 1H), 4.66-4.62 (m, 2H), 4.45-4.44 (br, 2H), 3.77-3.18 (m, 7H), 3.25-3.18 (m, 2H), 2.97-2.90 (m, 2H), 2.58-2.52 (m, 4H), 2.37-2.33 (br, 2H), 2.17 (s, 3H), 2.10-2.02 (m, 2H), 1.57-1.51 (m, 8H), 0.89 (t, J=9.6 Hz, 3H). LCMS (ESI) calcd, for C₃₄H₄₆N₅O₃⁺ [M+H]⁺: 611.4; found, 611.7.

### Intermediate Example 59: Preparation of N-((4,6-dimethyl-2-oxo-1,2-dihydropyridin-3-yl)methyl)-5-(ethyl(tetrahydro-2H-pyran-4-yl)amino)-4-methyl-4′-(piperazin-1-yl)-[1,1′-biphenyl]-3-carboxamide (GT-S-11)

N-((4,6-dimethyl-2-oxo-1,2-dihydropyridin-3-yl)methyl)-5-(ethyl(tetrahydro-2H-pyran-4-yl)amino)-4-methyl-4′-(piperazin-1-yl)-[1,1′-biphenyl]-3-carboxamide (GT-S-11) was prepared by referring to Scheme M-32 (white solid, 2.6 g). ¹H NMR (400 MHz, DMSO-d₆) δ 8.21 (t, J=6.0 Hz, 1H), 7.54 (d, J=11.6 Hz, 2H), 7.3 (s, 1H), 7.20 (s, 1H), 7.07 (d, J=11.2 Hz, 2H), 4.31 (d, J=6 Hz, 2H), 3.86 (d, J=13.6 Hz, 2H), 3.35-3.12 (m, 15 H), 2.24 (d, J=6.4 Hz, 6H), 2.14 (s, 2H), 1.71-1.48 (m, 4H), 0.86 (t, J=9.2 Hz, 3H). LCMS (ESI) calcd. for C₃₃H₄₄N₅O₃⁺ [M+H]⁺: 558.3; found, 558.5.

### Intermediate Example 60: Preparation of N-((4,6-dimethyl-2-oxo-1,2-dihydropyridin-3-yl)methyl)-5-(ethyl(tetrahydro-2H-pyran-4-yl)amino)-4-methyl-4′-(4-(methylamino)piperidin-1-yl)-[1,1′-biphenyl]-3-carboxamide (GT-S-12)

N-((4,6-dimethyl-2-oxo-1,2-dihydropyridin-3-yl)methyl)-5-(ethyl(tetrahydro-2H-pyran-4-yl)amino)-4-methyl-4′-(4-(methylamino)piperidin-1-yl)-[1,1′-biphenyl]-3-carboxamide (GT-S-12) was prepared by referring to Scheme M-33 (white solid, 1.8 g). ¹H NMR (400 MHz, DMSO-d₆) δ 8.16 (t, J=4.8 Hz, 1H), 7.45 (d, J=8.8 Hz, 2H), 7.33 (s, 1H), 7.16 (s, 1H), 6.98 (d, J=8.8 Hz, 2H), 5.86 (s, 1H), 4.29 (d, J=4.8 Hz, 2H), 3.82 (d, J=10.4 Hz, 2H), 3.65 (d, J=12.4 Hz, 2H), 3.25 (t, J=11.2 Hz, 3H), 3.10-3.01 (m, 4H), 2.76 (t, J=11.6 Hz, 2H), 2.45-2.38 (m, 1H), 2.29 (s, 3H), 2.21 (d, J=6.8 Hz, 6H), 2.11 (s, 3H), 1.89-1.85 (m, 2H), 1.66-1.64 (m, 2H), 1.55-1.49 (m, 2H), 1.32-1.28 (m, 2H), 0.83 (t, J=6.8 Hz, 3H). LCMS (ESI) calcd, for C₃₅H₄₈N₅O₃⁺ [M+H]⁺: 586.4; found, 586.2.

### Intermediate Example 61: Preparation of N-((4,6-dimethyl-2-oxo-1,2-dihydropyridin-3-yl)methyl)-5-(ethyl(tetrahydro-2H-pyran-4-yl)amino)-4-methyl-4′-(4-(piperidin-4-yl)piperazin-1-yl)-[1,1′-biphenyl]-3-carboxamide (GT-S-13)

N-((4,6-dimethyl-2-oxo-1,2-dihydropyridin-3-yl)methyl)-5-(ethyl(tetrahydro-2H-pyran-4-yl)amino)-4-methyl-4′-(4-(piperidin-4-yl)piperazin-1-yl)-[1,1′-biphenyl]-3-carboxamide (GT-S-13) was prepared by referring to Scheme M-34 (white solid, 1.6 g). ¹H NMR (400 MHz, DMSO-d₆) δ 8.18 (t, J=2.0 Hz, 1H), 7.47 (d, J=8.4 Hz, 2H), 7.33 (s, 1H), 7.16 (s, 1H), 6.98 (d, J=8.0 Hz, 2H), 5.86 (s, 1H), 4.29 (d, J=4.0 Hz, 2H), 4.14-4.06 (m, 1H), 3.82 (d, J=9.2 Hz, 2H), 3.41-3.36 (m, 2H), 3.29 (d, J=12.0 Hz, 3H), 3.14-2.98 (m, 9H), 3.62 (s, 4H), 2.28-2.21 (m, 7H), 2.11 (s, 3H), 1.68-1.64 (m, 4H), 1.53-1.50 (m, 2H), 1.26-1.24 (m, 2H), 0.83 (t, J=6.8 Hz, 3H). LCMS (ESI) calcd, for C₃₈H₅₃N₆O₃⁺ [M+H]⁺: 641.4; found, 642.0.

### Intermediate Example 62: Preparation of the Following Intermediate Compounds

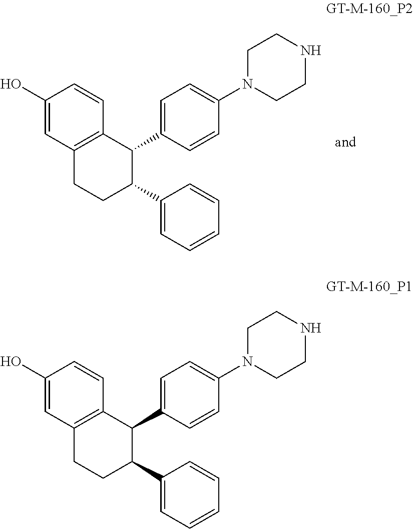 [SMILES: Oc1ccc2c(c1)CC[C@@H](c1ccccc1)[C@H]2c1ccc(N2CCNCC2)cc1 ; Oc1ccc2c(c1)CC[C@H](c1ccccc1)[C@@H]2c1ccc(N2CCNCC2)cc1]

Following the method of Scheme M-36, the enantiomeric mixture product obtained according to Step 8 of Scheme 49 was separated by supercritical fluid chromatography (SFC) to obtain the two intermediate compounds as described above (their retention times were 8.232 min and 9.400 min, respectively, in terms of analytical methods). The analytical method, preparation method, and conditions for SFC were shown below.

The analysis method and conditions for SFC are as follows:

| System: | Shimadzu LC-20AP | |
|---|---|---|
| Column name: | Daicel CHIRALPAK ®IE | |
| Column size: | 250*4.6 mm 5 μm | |
| Mobile Phase A: | n-Hexane | |
| Mobile Phase B: | EtOH (0.2% Diethylamine) | |
| Mobile Phase A: Mobile Phase B | 70:30 | |
| Wavelength: | 254 | nm |
| Flow | 1 | mL/min |
| Column temp: | 25 | °C |
| Injection: | 5 | μL |

| | |
|---|---|
| Solvent: | EtOH: HPLC grade |
| | n-Hexane: HPLC grade |

Preparation method for SFC is as follows:

| System: | Shimadzu | |
|---|---|---|
| Column name: | DAICELCHIRALPAK ®IE | |
| Column size: | 250*25 mm 10 μm | |
| Mobile Phase A: | n-Hexane | |
| Mobile Phase B: | ETOH (+0.1% 7.0 mol/l Ammonia in MeOH) | |
| A:B: | 70:30 | |
| Wavelength: | 214 | nm |
| Flow: | 30 | ml/min |
| Column temp: | rt | |
| Injection: | 1 | mL |
| Cycle time: | 12 | min |

| | |
|---|---|
| Solvent: | n-Hexane: redistilled grade |
| | ETOH: redistilled grade |
| Preparation of | The sample was dissolved in approximately |
| sample solution: | 30 mL of ethanol. |

One of the obtained intermediates (GT-M-160_P1) (white solid, 565 mg): ¹H NMR (400 MHz, DMSO) δ9.11 (s, 1H), 7.23-7.06 (m, 3H), 6.84 (d, J=6.7 Hz, 2H), 6.62 (dd, J=15.5, 5.3 Hz, 2H), 6.55-6.43 (m, 3H), 6.21 (d, J=8.6 Hz, 2H), 4.13 (d, J=4.9 Hz, 1H), 3.28 (s, 2H), 3.04-2.91 (m, 2H), 2.91-2.83 (m, 4H), 2.80-2.70 (m, 4H), 2.11 (dd, J=12.3, 6.4 Hz, 1H), 1.71 (d, J=7.5 Hz, 1H). LCMS (ESI) calcd, for C₂₆H₂₉N₂O⁺ [M+H]⁺: 385.23, found, 385.30. (retention time was 8.232 min in terms of analytical method).

Another intermediate (GT-M-160_P2) (white solid, 595 mg): ¹H NMR (400 MHz, DMSO) δ 9.10 (s, 1H), 7.14 (dd, J=15.2, 7.8 Hz, 3H), 6.84 (d, J=6.9 Hz, 2H), 6.66-6.59 (m, 2H), 6.50 (dd, J=14.1, 8.3 Hz, 3H), 6.22 (d, J=8.4 Hz, 2H), 4.14 (d, J=4.6 Hz, 1H), 3.30-3.23 (m, 2H), 2.97 (dd, J=15.6, 9.6 Hz, 2H), 2.90 (d, J=5.0 Hz, 4H), 2.80 (d, J=4.5 Hz, 4H), 2.11 (dd, J=12.2, 6.8 Hz, 1H), 1.72 (s, 1H). LCMS (ESI) calcd. for C₂₆H₂₉N₂O⁺ [M+H]⁺: 385.23, found, 385.30. (retention time was 9.400 min in terms of analytical method).

### Intermediate Example 63: Preparation of the Following Intermediate Compounds

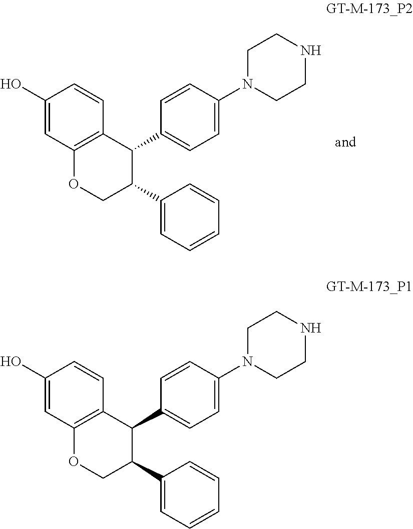 [SMILES: Oc1ccc2c(c1)OC[C@@H](c1ccccc1)[C@H]2c1ccc(N2CCNCC2)cc1 ; Oc1ccc2c(c1)OC[C@H](c1ccccc1)[C@@H]2c1ccc(N2CCNCC2)cc1]

Following the method of Scheme M-37, the enantiomeric mixture product obtained according to Step 6 of Scheme M-37 was separated by supercritical fluid chromatography (SFC) to obtain the two intermediate compounds as described above (their retention times were 1.763 min and 3.506 min, respectively, in terms of analytical methods). The analytical method, preparation method, and conditions for SFC were shown below.

The analysis method and conditions for SFC are as follows:

| System: | Waters Ultra Performance Convergence | | |
|---|---|---|---|
| | Chromatography (UPCC) (CA-185) | | |
| Column name: | DAICELCHIRALPAK ®OD | | |
| Column size: | 100*3.0 mm *3.0 μm | | |
| Mobile Phase A: | Supercritical CO₂ | | |
| Mobile Phase B: | Isopropanol (0.1% Diethylamine) | | |
| Wavelength: | 214 nm | | |
| Flow | 1.5 mL/min | | |
| Column temp: | 35 °C | | |
| Back Pressure(psi): | 1800 psi | | |
| Injection: | 0.3 μL | | |
| Run time: | 8 min | | |
| | Time (min) | A(%V/V) | B(%V/V) |
| Gradient | 0.00 | 60 | 40 |
| | 8.00 | 60 | 40 |

| | |
|---|---|
| Solvent: | Isopropanol: HPLC grade |
| | Supercritical CO₂: Food grade |
| Preparation of | Mobile Phase B: Add 1 mL Diethylamine in |
| mobile phases: | 1000 mL Isopropanol, then ultrasonic |
| | degassing for 15 min. |

Preparation method for SFC is as follows:

| System: | Waters SFC 150 | |
|---|---|---|
| Column name: | DAICELCHIRALCEL ®OD | |
| Column size: | 250*25 mm 10 μm | |
| Mobile Phase A: | Supercritical CO₂, | |
| Mobile Phase B: | Isopropanol (+0.1% 7.0 mol/l Ammonia in MEOH) | |
| A:B: | 45:55 | |
| Wavelength: | 214 | nm |
| Flow: | 80 | ml/min |
| Column temp: | RT | |
| Back Pressure: | 100 | bar |
| Injection: | 1.5 | mL |
| Cycle time: | 6 | min |

| | |
|---|---|
| Solvent: | Isopropanol: redistilled grade |
| | Supercritical CO₂: Food grade |

One of the obtained intermediates (GT-M-173_P1) (white solid, 555 mg): ¹H NMR (400 MHz, DMSO) δ9.33 (s, 1H), 7.22 (dd, J=20.1, 17.2 Hz, 3H), 6.88 (d, J=45.0 Hz, 2H), 6.71 (d, J=8.3 Hz, 1H), 6.64 (d, J=8.6 Hz, 2H), 6.44 (d, J=8.5 Hz, 2H), 6.39-6.22 (m, 2H), 4.39 (t, J=11.1 Hz, 1H), 4.30-4.06 (m, 2H), 3.55 (dt, J=39.1, 19.2 Hz, 1H), 2.95 (d, J=4.8 Hz, 4H), 2.83 (d, J=4.5 Hz, 4H). LCMS (ESI) calcd. for C₂₅H₂₇N₂O₂⁺ [M+H]⁺: 387.21, found, 387.40. (retention time was 1.763 min in terms of analytical method).

Another intermediate (GT-M-173_P2) (white solid, 495 mg): ¹H NMR (400 MHz, DMSO) δ 9.33 (s, 1H), 7.22 (dd, J=20.1, 17.2 Hz, 3H), 6.88 (d, J=45.0 Hz, 2H), 6.71 (d, J=8.3 Hz, 1H), 6.64 (d, J=8.6 Hz, 2H), 6.44 (d, J=8.5 Hz, 2H), 6.39-6.22 (m, 2H), 4.39 (t, J=11.1 Hz, 1H), 4.30-4.06 (m, 2H), 3.55 (dt, J=39.1, 19.2 Hz, 1H), 2.95 (d, J=4.8 Hz, 4H), 2.83 (d, J=4.5 Hz, 4H). LCMS (ESI) calcd, for C₂₅H₂₇N₂O₂⁺ [M+H]⁺: 387.21, found, 387.40. (retention time was 3.506 min in terms of analytical method).

### Intermediate Example 64: Preparation of N-(3-(difluoromethyl)-1-(1-methylpiperidin-4-yl)-1H-pyrazol-4-yl)-5-morpholinopyrazolo[1,5-a]pyrimidine-3-carboxamide (GT-04349)

The target compound (GT-04349) was prepared by referring to Scheme M-35 (white solid, 15 mg, yield 67.08%). ¹H NMR (400 MHz, MeOD) δ 8.45 (d, J=8.0 Hz, 1H), 8.33 (s, 1H), 8.22 (s, 1H), 6.97-6.59 (m, 2H), 4.50 (s, 2H), 3.69 (dd, J=35.3, 32.6 Hz, 10H), 3.16 (s, 1H), 2.85 (s, 3H), 2.29 (s, 4H). LCMS (ESI) calcd. for C₂₁H₂₇F₂N₈₀₂⁺ [M+H]⁺: 461.22, found, 461.2.

### Intermediate Example 65: Preparation of 2-(6,7-dihydro-5H-pyrrolo[1,2-c]imidazol-1-yl)-2-(4-fluoro-1-oxo-6-(4-(4-(piperazin-1-yl)piperidin-1-yl)phenyl)isoindolin-2-yl)-N-(thiazol-2-yl)acetamide (GT-D-219)

The target compound GT-D-219 was prepared by referring to Scheme M-38 (yellow solid, 1.1 g, yield 113.53%). ¹H NMR (400 MHz, MeOD) δ 8.92 (s, 1H), 8.03-7.82 (m, 5H), 7.77 (dd, J=10.0, 1.2 Hz, 1H), 7.58 (d, J=4.0 Hz, 1H), 7.34 (d, J=4.0 Hz, 1H), 4.95 (d, J=17.2 Hz, 2H), 4.65 (d, J=17.2 Hz, 1H), 4.44-4.29 (m, 2H), 4.00 (d, J=12.0 Hz, 3H), 3.92-3.65 (m, 10H), 3.10-2.97 (m, 1H), 2.94-2.82 (m, 1H), 2.78-2.48 (m, 6H). LCMS (ESI) calcd, for C₃₄H₃₈FN₈O₂S⁺ [M+H]⁺: 641.28, found, 641.4.

### Intermediate Example 66: Preparation of 2-(6,7-dihydro-5H-pyrrolo[1,2-c]imidazol-1-yl)-2-(4-fluoro-1-oxo-6-(4-(4-(piperidin-4-yl)piperazin-1-yl)phenyl)isoindolin-2-yl)-N-(thiazol-2-yl)acetamide (GT-D-217)

The target compound GT-D-217 was prepared by referring to Scheme M-39 (yellow solid, 800 mg, yield 99.45%). ¹H NMR (400 MHz, MeOD) δ 8.90 (s, 1H), 7.88 (s, 1H), 7.66 (d, J=8.6 Hz, 3H), 7.57-7.51 (m, 1H), 7.32-7.26 (m, 1H), 7.18-6.89 (m, 3H), 4.91 (d, J=16.0 Hz, 1H), 4.82-4.75 (m, 1H), 4.58 (d, J=17.0 Hz, 1H), 4.38-4.33 (m, 2H), 4.01 (s, 2H), 3.78 (s, 2H), 3.64 (d, J=12.0 Hz, 2H), 3.38 (s, 2H), 3.26 (s, 2H), 3.18-3.12 (m, 2H), 3.07-2.99 (m, 1H), 2.91-2.84 (m, 1H), 2.76-2.63 (m, 2H), 2.52 (d, J=12.0 Hz, 2H), 2.18-2.05 (m, 2H). LCMS (ESI) calcd, for C₃₄H₃₈FN₈O₂S⁺ [M+H]⁺: 641.28, found, 641.4.

### Intermediate Example 67: Preparation of 2-(6-(4-((1S,4S)-2,5-diazabicyclo[2.2.1]heptan-2-yl)phenyl)-4-fluoro-1-oxoisoindolin-2-yl)-2-(6,7-dihydro-5H-pyrrolo[1,2-c]imidazol-1-yl)-N-(thiazol-2-yl)acetamide (GT-D-222)

Following steps 2-5 in Scheme M-38, the reaction of the starting materials ethyl 2-(6-bromo-4-fluoro-1-oxoisoindolin-2-yl)-2-(6,7-dihydro-5H-pyrrolo[1,2-c]imidazol-1-yl)acetate (CAS NO.: 2407965-04-0) and (1S,4S)-tert-butyl 5-(4-(4,4,5,5-tetramethyl-1,3,2-dioxaborolan-2-yl)phenyl)-2,5-diazabicyclo[2.2.1]heptane-2-carboxylate (CAS NO.: 942189-80-2) afforded the target compound GT-D-222 (off-white solid, 0.56 g, yield 109.7%). ¹H NMR (400 MHz, MeOD) δ 8.90 (s, 1H), 7.85 (d, J=1.2 Hz, 1H), 7.69-7.59 (m, 3H), 7.54 (d, J=4.0 Hz, 1H), 7.29 (d, J=4.0 Hz, 1H), 6.81 (d, J=8.0 Hz, 2H), 4.91 (d, J=10.0 Hz, 1H), 4.87-4.81 (m, 1H), 4.74 (s, 1H), 4.62-4.50 (m, 2H), 4.35 (dd, J=12.0, 7.2 Hz, 2H), 3.79 (dd, J=10.0, 2.4 Hz, 1H), 3.40 (s, 2H), 3.06-2.81 (m, 2H), 2.77-2.61 (m, 2H), 2.33 (d, J=10.0 Hz, 1H), 2.09 (d, J=12.0 Hz, 1H), 2.00 (d, J=8.8 Hz, 1H), 1.60 (s, 1H). LCMS (ESI) calcd, for C₃₀H₂₉FN₇O₂S⁺ [M+H]⁺: 570.21, found, 570.2.

### Intermediate Example 68: Preparation of 2-(6,7-dihydro-5H-pyrrolo[1,2-c]imidazol-1-yl)-2-(4-fluoro-6-(4-(4-(methylamino)piperidin-1-yl)phenyl)-1-oxoisoindolin-2-yl)-N-(thiazol-2-yl)acetamide (GT-D-221)

Following steps 2-5 in Scheme M-38, the reaction of the starting materials ethyl 2-(6-bromo-4-fluoro-1-oxoisoindolin-2-yl)-2-(6,7-dihydro-5H-pyrrolo[1,2-c]imidazol-1-yl)acetate and tert-butyl (1-(4-(4,4,5,5-tetramethyl-1,3,2-dioxaborolan-2-yl)phenyl)piperidin-4-yl) carbamate (CAS No.: 2235416-80-3) afforded the target compound GT-D-221 (yellow solid, 0.72 g, yield 105.4%). ¹H NMR (400 MHz, MeOD) δ 8.91 (s, 1H), 7.96 (d, J=1.2 Hz, 1H), 7.92 (d, J=3.2 Hz, 3H), 7.77 (dd, J=10.0, 1.2 Hz, 1H), 7.55 (d, J=3.6 Hz, 1H), 7.30 (d, J=3.6 Hz, 1H), 6.51 (s, 1H), 4.96 (d, J=17.2 Hz, 1H), 4.88 (s, 1H), 4.83 (s, 1H), 4.63 (d, J=17.2 Hz, 1H), 4.34 (dt, J=12.0, 7.2 Hz, 2H), 3.93 (d, J=12.0 Hz, 2H), 3.85 (t, J=11.2 Hz, 2H), 3.63 (dd, J=20.0, 9.6 Hz, 1H), 3.08-2.98 (m, 1H), 2.91-2.83 (m, 1H), 2.81 (s, 3H), 2.74-2.63 (m, 2H), 2.51 (d, J=12.0 Hz, 2H), 2.44-2.31 (m, 2H). LCMS (ESI) calcd, for C₃₁H₃₃FN₇O₂S⁺ [M+H]⁺: 586.24, found, 586.3.

### Intermediate Example 69: Preparation of 2-(6-(4-(3,8-diazabicyclo[3.2.1]octan-3-yl)phenyl)-4-fluoro-1-oxoisoindolin-2-yl)-2-(6,7-dihydro-5H-pyrrolo[1,2-c]imidazol-1-yl)-N-(thiazol-2-yl)acetamide (GT-D-224)

With reference to Scheme M-38, replacement of the Step 1 compound (CAS No.: 2762613-62-5) with the starting compound 3-(4-bromophenyl)-3,8-diazabicyclo[3.2.1]octane-8-carboxylate (CAS No.: 1146427-86-2) afforded the target compound GT-D-224 as an off-white solid (619 mg, yield 105.0%). ¹H NMR (400 MHz, MeOD) δ 8.90 (s, 1H), 7.88 (d, J=1.0 Hz, 1H), 7.65 (dd, J=10.0, 7.2 Hz, 3H), 7.50 (d, J=4.0 Hz, 1H), 7.24 (d, J=4.0 Hz, 1H), 7.07 (d, J=8.0 Hz, 2H), 4.91 (d, J=16.0 Hz, 1H), 4.55 (d, J=16.0 Hz, 1H), 4.35 (dd, J=12.0, 8.0 Hz, 2H), 4.22 (s, 2H), 4.10 (q, J=7.2 Hz, 1H), 3.80 (d, J=10.0 Hz, 2H), 3.16 (t, J=12.0 Hz, 2H), 3.09-2.94 (m, 1H), 2.92-2.77 (m, 1H), 2.76-2.57 (m, 2H), 2.16 (s, 4H). LCMS (ESI) calcd, for C₃₁H₃₁FN₇O₂S⁺ [M+H]⁺: 584.22, found, 584.2.

### Intermediate Example 70: Preparation of 2-(6-(4-((1R,4R)-2,5-diazabicyclo[2.2.1]heptan-2-yl)phenyl)-4-fluoro-1-oxoisoindolin-2-yl)-2-(6,7-dihydro-5H-pyrrolo[1,2-c]imidazol-1-yl)-N-(thiazol-2-yl)acetamide (GT-D-223)

With reference to Steps 2-5 of Scheme M-38, the reaction of the starting compounds ethyl 2-(6-bromo-4-fluoro-1-oxoisoindolin-2-yl)-2-(6,7-dihydro-5H-pyrrolo[1,2-c]imidazol-1-yl)acetate (CAS No.: 2407965-04-0) and (1R,4R)-tert-butyl 5-(4-(4,4,5,5-tetramethyl-1,3,2-dioxaborolan-2-yl)phenyl)-2,5-diazabicyclo[2.2.1]heptane-2-carboxylate (CAS No.: 2654825-27-9) afforded the target compound GT-D-223 as an off-white solid (1.3 g, 101.7% yield). ¹H NMR (400 MHz, MeOD) δ 8.90 (s, 1H), 7.84 (d, J=4.0 Hz, 1H), 7.64-7.58 (m, 4H), 7.35 (d, J=4.0 Hz, 1H), 6.81 (d, J=8.7 Hz, 2H), 4.92 (s, 1H), 4.87-4.84 (m, 1H), 4.74 (s, 1H), 4.67-4.48 (m, 2H), 4.39-4.32 (m, 2H), 3.80-3.77 (m, 1H), 3.40 (s, 3H), 3.11-2.96 (m, 1H), 2.95-2.82 (m, 1H), 2.72-2.66 (m, 2H), 2.32 (d, J=8.0 Hz, 1H), 2.10 (d, J=12.0 Hz, 1H). LCMS (ESI) calcd, for C₃₀H₂₉FN₇O₂S⁺ [M+H]⁺: 570.21, found, 570.2.

### Intermediate Example 71: Preparation of 2-(6-(4-(4-(3,3-difluoropiperidin-4-yl)piperazin-1-yl)phenyl)-4-fluoro-1-oxoisoindolin-2-yl)-2-(6,7-dihydro-5H-pyrrolo[1,2-c]imidazol-1-yl)-N-(thiazol-2-yl)acetamide (GT-D-218)

With reference to Steps 3-6 of Scheme M-39, the reaction of the starting compounds ethyl 2-(6,7-dihydro-5H-pyrrolo[1,2-c]imidazol-1-yl)-2-(4-fluoro-1-oxo-6-(4-(piperazin-1-yl)phenyl)isoindolin-2-yl)acetate (Compound 2) and tert-butyl 3,3-difluoro-4-oxopiperidine-1-carboxylate afforded the target compound GT-D-218 (off-white solid, 645 mg, yield 103.32%). ¹H NMR (400 MHz, MeOD) δ 8.92 (s, 1H), 7.93 (d, J=1.2 Hz, 1H), 7.84 (d, J=8.8 Hz, 2H), 7.74 (dd, J=10.0, 1.2 Hz, 1H), 7.69-7.55 (m, 3H), 7.42 (d, J=4.0 Hz, 1H), 4.95 (s, 2H), 4.68 (d, J=16.0 Hz, 1H), 4.45-4.24 (m, 2H), 3.88 (t, J=8.0 Hz, 2H), 3.76 (s, 4H), 3.67-3.49 (m, 6H), 3.33 (d, J=12.0 Hz, 1H), 3.10-2.96 (m, 1H), 2.96-2.81 (m, 1H), 2.77-2.59 (m, 2H), 2.49 (d, J=12.0 Hz, 1H), 2.37-2.18 (m, 1H). LCMS (ESI) calcd, for C₃₄H₃₆F₃N₈O₂S⁺ [M+H]⁺: 677.26, found, 677.3.

### Intermediate Example 72: Preparation of N-(5-(2-hydroxypropan-2-yl)-2-(piperidin-4-yl)benzo[d]oxazol-6-yl)-6-(trifluoromethyl) picolinamide (GT-D-208)

With reference to Scheme M-40, the target compound GT-D-208 was obtained as a yellow solid (1.7 g, 109% yield). ¹H NMR (400 MHz, MeOD) δ 8.79 (s, 1H), 8.48 (d, J=8.0 Hz, 1H), 8.30 (t, J=8.0 Hz, 1H), 8.04 (d, J=7.6 Hz, 1H), 7.71 (s, 1H), 3.56-3.48 (m, 3H), 3.47-3.41 (m, 1H), 3.28-3.20 (m, 2H), 2.45 (dd, J=14.4, 3.6 Hz, 2H), 2.23-2.11 (m, 2H), 1.71 (s, 6H). LCMS (ESI) calcd, for C₂₂H₂₄F₃N₄₀₃⁺ [M+H]⁺: 449.18, found, 449.2.

### Intermediate Example 73: Preparation of N-(6-(2-hydroxypropan-2-yl)-2-(piperidin-4-yl)-2H-indazol-5-yl)-6-(trifluoromethyl) picolinamide (GT-D-210)

With reference to Scheme M-41, the target compound GT-D-210 was obtained as a yellow solid (1.52 g, yield 109.4%). ¹H NMR (400 MHz, D₂O) δ 8.20 (s, 1H), 8.10 (s, 1H), 8.04 (d, J=4.0 Hz, 2H), 7.85-7.79 (m, 1H), 7.49 (s, 1H), 4.67-4.62 (m, 1H), 3.57 (d, J=13.2 Hz, 2H), 3.20 (dd, J=12.8, 10.0 Hz, 2H), 2.34-2.14 (m, 4H), 1.54 (s, 6H). LCMS (ESI) calcd, for C₂₂H₂₅F₃N₅O₂⁺ [M+H]⁺: 448.20, found, 448.2.

### Intermediate Example 74: Preparation of 2-(6-(4-(3,3-difluoro-4-(piperazin-1-yl)piperidin-1-yl)phenyl)-4-fluoro-1-oxoisoindolin-2-yl)-2-(6,7-dihydro-5H-pyrrolo[1,2-c]imidazol-1-yl)-N-(thiazol-2-yl)acetamide (GT-D-220)

With reference to Scheme M-42, the target compound GT-D-220 was obtained as a brown solid (0.5 g, yield 66.6%). ¹H NMR (400 MHz, MeOD) δ 7.83 (s, 1H), 7.65 (s, 1H), 7.60-7.58 (m, 3H), 7.44 (d, J=3.6 Hz, 1H), 7.15 (d, J=4.0 Hz, 1H), 7.06 (d, J=8.0 Hz, 2H), 4.85 (s, 1H), 4.81 (s, 1H), 4.25 (d, J=16.0 Hz, 1H), 4.12-4.04 (m, 2H), 3.98-3.91 (m, 2H), 3.19 (d, J=4.0 Hz, 4H), 3.16-2.76 (m, 8H), 2.65-2.56 (m, 3H), 2.11-2.06 (m, 1H), 2.00-1.86 (m, 1H). LCMS (ESI) calcd, for C₃₄H₃₆F₃N₈O₂S⁺ [M+H]⁺: 677.26, found, 677.4.

### Intermediate Example 75: Preparation of N-(6-methoxy-2-(piperidin-4-yl)-2H-indazol-5-yl)-6-(trifluoromethyl) picolinamide (GT-D-209)

With reference to Scheme M-43, the target compound GT-D-209 was obtained as a yellow solid (4.2 g, yield 89.70%). ¹H NMR (400 MHz, MeOD) δ 8.82 (s, 1H), 8.78 (s, 1H), 8.40 (d, J=7.8 Hz, 1H), 8.29 (t, J=7.8 Hz, 1H), 8.04 (d, J=7.8 Hz, 1H), 7.11 (s, 1H), 5.19-5.05 (m, 1H), 4.09 (s, 3H), 3.71 (d, J=13.2 Hz, 2H), 3.45-3.34 (m, 2H), 2.71-2.44 (m, 4H). LCMS (ESI) calcd, for C₂₀H₂₁F₃N₅O₂⁺ [M+H]⁺: 420.16, found, 420.2.

### Intermediate Example 76: N-(6-methoxy-2-(piperidin-4-yl)-2H-indazol-5-yl)-3-(trifluoromethyl)benzamide (GT-D-225)

With reference to Scheme M-43, the target compound GT-D-225 was obtained as a white solid (1.8 g, yield 92.95%). ¹H NMR (400 MHz, MeOD) δ 8.58 (s, 1H), 8.53 (s, 1H), 8.27 (s, 1H), 8.23 (d, J=8.0 Hz, 1H), 7.94 (d, J=7.6 Hz, 1H), 7.78 (t, J=7.6 Hz, 1H), 7.17 (s, 1H), 5.03-4.94 (m, 1H), 4.07 (s, 3H), 3.68 (d, J=13.2 Hz, 2H), 3.39-3.34 (m, 2H), 2.71-2.30 (m, 4H). LCMS (ESI) calcd, for C₂₁H₂₂F₃N₄₀₂⁺ [M+H]⁺: 419.17, found, 419.0.

### Intermediate Example 77: N-(1-((1s,4s)-4-(hydroxymethyl)cyclohexyl)-5-(4-(piperidin-4-yl)piperazin-1-yl)-1H-benzo[d]imidazol-2-yl)-3-(trifluoromethyl)benzamide (GT-D-131)

With reference to Scheme M-44, the target compound GT-D-131 was obtained as a brown solid (410 mg, yield 42.96%). ¹H NMR (400 MHz, DMSO) δ 8.49 (d, J=7.6 Hz, 1H), 8.44 (s, 1H), 7.88 (d, J=7.8 Hz, 1H), 7.73 (t, J=7.6 Hz, 1H), 7.43 (d, J=8.8 Hz, 1H), 7.15 (d, J=2.2 Hz, 1H), 6.94 (dd, J=8.8, 2.4 Hz, 1H), 4.69 (s, 1H), 4.56 (s, 1H), 4.43 (s, 1H), 3.68 (s, 2H), 3.39 (s, 2H), 3.07 (d, J=5.2 Hz, 4H), 3.03 (d, J=12.8 Hz, 2H), 2.66 (d, J=4.4 Hz, 4H), 2.46 (d, J=11.6 Hz, 2H), 2.32 (d, J=3.6 Hz, 1H), 1.94 (d, J=13.2 Hz, 2H), 1.85 (s, 1H), 1.76 (d, J=11.2 Hz, 2H), 1.68 (d, J=9.6 Hz, 1H), 1.61 (d, J=10.4 Hz, 2H), 1.33 (dt, J=11.2, 9.6 Hz, 2H). LCMS (ESI) calcd, for C₃₁H₄₀F₃N₆O₂⁺ [M+H]⁺: 585.32, found, 585.2.

### Intermediate Example 78: N-(1-((1s,4s)-4-(hydroxymethyl)cyclohexyl)-5-(piperazin-1-yl)-1H-benzo[d]imidazol-2-yl)-3-(trifluoromethyl)benzamide (GT-D-129)

With reference to Steps 1-6 and Step 9 of Scheme M-44, the target compound GT-D-129 was obtained as a yellow solid (1 g, yield 42.23%). ¹H NMR (400 MHz, DMSO) δ 8.61-8.42 (m, 2H), 7.94 (s, 1H), 7.80 (s, 1H), 7.51 (d, J=8.0 Hz, 1H), 7.22 (s, 1H), 7.01 (d, J=7.6 Hz, 1H), 4.76-4.64 (m, 2H), 3.74 (s, 2H), 3.11 (s, 4H), 2.99 (s, 4H), 2.05-1.91 (m, 4H), 1.69 (s, 4H). LCMS (ESI) calcd, for C₂₆H₃₁F₃N₅O₂⁺ [M+H]⁺: 502.24, found, 502.3.

### Intermediate Example 79: Preparation of 2-((5-bromo-2-((2-methyl-4-(piperazin-1-ylsulfonyl)phenyl)amino)pyrimidin-4-yl)amino)-6-fluorobenzamide

With reference to Scheme M-45, the target compound was obtained (722 mg, yield 33%). ¹H NMR (300 MHz, DMSO-d₆) δ 10.03 (s, 1H), 8.14 (dd, J=19.3, 6.7 Hz, 4H), 7.51 (q, J=7.8 Hz, 1H), 7.26 (d, J=6.5 Hz, 2H), 7.01 (t, J=9.3 Hz, 1H), 6.68 (d, J=8.3 Hz, 1H), 6.10-5.52 (m, 2H), 3.74 (t, J=4.8 Hz, 4H), 2.85 (d, J=5.4 Hz, 4H), 2.08 (s, 3H). LCMS (ESI): calcd, for [M+H]⁺: 564.08; found, 564.1.

### Intermediate Example 80: Preparation of (5-amino-3-((4-(piperazin-1-ylsulfonyl)phenyl)amino)-1H-1,2,4-triazol-1-yl) (2,6-difluorophenyl)methanone

With reference to Scheme M-46, the target compound was obtained (252 mg, yield 88%). ¹H NMR (400 MHz, DMSO-d₆) δ 10.08 (s, 1H), 9.18 (s, 2H), 8.05 (s, 1H), 7.73 (ddd, J=15.2, 8.6, 6.6 Hz, 1H), 7.56 (q, J=9.0 Hz, 4H), 7.35 (t, J=8.3 Hz, 2H), 3.13 (q, J=4.7 Hz, 4H), 3.10-3.04 (m, 4H). LCMS (ESI): calcd. for [M+H]⁺: 464.13; found, 464.8.

### Intermediate Example 81: Preparation of N-((4-(((R)-4-((1R,4R)-2,5-diazabicyclo[2.2.1]heptan-2-yl)-1-(phenylthio) butan-2-yl)amino)-3-((trifluoromethyl) sulfonyl)phenyl) sulfonyl)-4-(4-((4′-chloro-4,4-dimethyl-3,4,5,6-tetrahydro-[1,1′-biphenyl]-2-yl)methyl)piperazin-1-yl)benzamide

With reference to Scheme M-47, the target compound was obtained (460 mg, yield 50.67%). ¹H NMR (400 MHz, DMSO-d₆) δ 10.08 (s, 1H), 9.18 (s, 2H), 8.05 (s, 1H), 7.73 (ddd, J=15.2, 8.6, 6.6 Hz, 1H), 7.56 (q, J=9.0 Hz, 4H), 7.35 (t, J=8.3 Hz, 2H), 3.13 (q, J=4.7 Hz, 4H), 3.10-3.04 (m, 4H). LCMS (ESI): calcd. for [M+H]: 985.6; found, 985.1.

### Intermediate Example 82: Preparation of 4-(2-hydroxy-5-(methylsulfonyl)phenyl)-6-methyl-1,6-dihydro-7H-pyrrolo[2,3-c]pyridin-7-one (GTC00392)

With reference to Scheme M-48, the target compound GTC00392 was obtained (white solid, 1.2 g). ¹H NMR (400 MHz, MeOD) δ 7.91 (d, J=2.4 Hz, 1H), 7.79 (dd, J=8.4, 2.4 Hz, 1H), 7.35-7.31 (m, 2H), 7.12 (d, J=8.4 Hz, 1H), 6.27 (d, J=2.8 Hz, 1H), 3.70 (s, 3H), 3.11 (d, J=4.4 Hz, 3H). LCMS (ESI) calcd. 319.07; found, 319.0.

### Intermediate Example 83: Preparation of 1-(4-((5-(3,5-dimethylisoxazol-4-yl)-2-methylphenyl) (piperidin-4-ylmethyl)amino)phenyl)cyclopropane-1-carbonitrile (GTC00388)

With reference to Scheme M-49, the target compound GTC00388 was obtained (yellow solid, 2.5 g, yield 80.74%). ¹H NMR (400 MHz, MeOD) δ 7.43 (d, J=7.6 Hz, 1H), 7.22 (d, J=9.6 Hz, 2H), 7.15 (d, J=8.8 Hz, 2H), 6.57 (d, J=8.8 Hz, 2H), 3.63 (d, J=6.4 Hz, 2H), 3.49-3.33 (m, 2H), 2.96 (dt, J=13.2, 6.4 Hz, 2H), 2.42 (s, 3H), 2.26 (s, 3H), 2.19-2.02 (m, 6H), 1.65-1.54 (m, 2H), 1.55-1.42 (m, 2H), 1.38-1.29 (m, 2H). LCMS (ESI) calcd. 441.26; found, 441.2.

### Intermediate Example 84: Preparation of 5-chloro-4-(5-(cyclopropylmethyl)-1-methyl-1H-pyrazol-4-yl)-N-(piperidin-4-yl)pyrimidin-2-amine hydrochloride (GTC00324)

With reference to Scheme M-50, the target compound GTC00324 was obtained (white-like solid, 2.49 g, yield 96.89%). ¹H NMR (400 MHz, DMSO) δ 9.18 (s, 1H), 8.97 (s, 1H), 8.21 (d, J=10.4 Hz, 1H), 7.88 (s, 1H), 7.64 (s, 1H), 3.87 (d, J=22.7 Hz, 1H), 3.78-3.66 (m, 3H), 3.16 (d, J=12.4 Hz, 2H), 2.92 (d, J=6.4 Hz, 2H), 2.79 (d, J=10.2 Hz, 2H), 1.88 (t, J=11.4 Hz, 2H), 1.63 (dd, J=21.1, 10.3 Hz, 2H), 0.82 (s, 1H), 0.25 (d, J=6.7 Hz, 2H), −0.00 (s, 2H). LCMS (ESI) calcd. 347.17; found, 347.2.

### Intermediate Example 85: Preparation of 2-fluoro-5-methoxy-4-((4-((2-methyl-3-oxoisoindolin-4-yl)oxy)-5-(trifluoromethyl)pyrimidin-2-yl)amino)-N-(piperidin-4-yl)benzamide (GTC00226)

With reference to Scheme M-51, the target compound GTC00226 was obtained (light yellow solid, 595 mg, yield 87.33%). ¹H NMR (400 MHz, MeOD) δ 8.59 (d, J=0.7 Hz, 1H), 7.71 (t, J=7.8 Hz, 1H), 7.54 (d, J=7.5 Hz, 1H), 7.47 (d, J=12.8 Hz, 1H), 7.26 (dd, J=18.3, 7.2 Hz, 2H), 4.52 (s, 2H), 4.19-4.11 (m, 1H), 3.93 (s, 3H), 3.49-3.44 (m, 2H), 3.17 (dt, J=11.7, 5.8 Hz, 2H), 3.03 (s, 3H), 2.21 (dd, J=13.7, 2.6 Hz, 2H), 1.85 (dt, J=14.3, 7.4 Hz, 2H). LCMS (ESI) calcd. 575.20; found, 575.2.

### Intermediate Example 86: Preparation of (R)—N-(5-(3-hydroxypyrrolidin-1-yl)-2-(piperazin-1-yl) oxazolo[4,5-b]pyridin-6-yl)-2-(2-methylpyridin-4-yl) oxazole-4-carboxamide (GTC00176)

With reference to Scheme M-52, the target compound GTC00176 was obtained (white solid, 1.6 g). ¹H NMR (400 MHz, DMSO) δ 9.85 (s, 1H), 8.98 (s, 1H), 8.69 (d, J=5.2 Hz, 1H), 7.87 (s, 1H), 7.78 (d, J=5.1 Hz, 1H), 7.70 (s, 1H), 4.89 (d, J=3.3 Hz, 1H), 4.29 (s, 1H), 3.89-3.82 (m, 4H), 3.69-3.53 (m, 2H), 3.48-3.40 (m, 1H), 3.29-3.15 (m, 6H), 2.62 (d, J=17.3 Hz, 3H), 1.87 (ddd, J=49.9, 9.2, 4.4 Hz, 2H). LCMS (ESI) calcd. 491.22; found, 491.2.

### Intermediate Example 87: Preparation of (R)-9-methyl-6-oxo-N-(1-(4-(piperazin-1-yl)benzyl)-1H-pyrazol-4-yl)-6,7,8,9-tetrahydropyrido[3′,2′: 4,5]pyrrolo[1,2-a]pyrazine-2-carboxamide (GTC00397)

With reference to Scheme M-53, the target compound GTC00397 was obtained (white solid, 2.0 g). ¹H NMR (400 MHz, MeOD) δ 8.45 (s, 1H), 8.29-8.20 (m, 2H), 8.00 (d, J=8.0 Hz, 1H), 7.35 (d, J=8.0 Hz, 2H), 7.23 (s, 1H), 7.15 (d, J=8.0 Hz, 2H), 5.44 (s, 2H), 5.41-5.34 (m, 1H), 3.99 (dd, J=12.0, 4.0 Hz, 1H), 3.58 (d, J=12.8 Hz, 1H), 3.54-3.49 (m, 4H), 3.42 (dd, J=6.0, 3.6 Hz, 4H), 1.50 (d, J=6.0 Hz, 3H). LCMS (ESI) calcd. 485.24; found, 485.2.

### Intermediate Example 88: Preparation of 4-((7H-pyrrolo[2,3-d]pyrimidin-4-yl)amino)-N-(4-(4-(piperazin-1-yl)piperidin-1-yl)phenyl)-1H-pyrazole-3-carboxamide (GTC00865)

With reference to Scheme M-55, the target compound GTC00865 was obtained (white solid, 1.8 g). ¹H NMR (300 MHz, CD₃OD) δ 8.25 (d, J=9.8 Hz, 2H), 7.92 (d, J=9.1 Hz, 2H), 7.69 (d, J=9.2 Hz, 2H), 7.45 (d, J=3.6 Hz, 1H), 6.89 (s, 1H), 3.88 (d, J=12.6 Hz, 2H), 3.78 (d, J=12.9 Hz, 3H), 3.71 (d, J=17.0 Hz, 8H), 2.57 (d, J=12.0 Hz, 2H), 2.52-2.38 (m, 2H). LCMS (ESI) calcd. 487.27, found, 487.2.

### Intermediate Example 89: Preparation of 7-(6-(piperazin-1-yl)pyridin-3-yl)-5H-pyrido[4,3-blindole (GTC00688)

With reference to Scheme M-56, the target compound GTC00688 was obtained (off-white solid, 0.95 g). ¹H NMR (400 MHz, MeOD) δ 9.55 (s, 1H), 8.59 (d, J=2.0 Hz, 1H), 8.55 (d, J=6.0 Hz, 1H), 8.46-8.40 (m, 1H), 8.18-8.15 (m, 1H), 8.00-7.92 (m, 2H), 7.82-7.73 (m, 1H), 7.18 (d, J=8.0 Hz, 1H), 3.95-3.93 (m, 4H), 3.41-3.38 (m, 4H). LCMS (ESI) calcd. 330.17; found, 330.2.

### Intermediate Example 90: Preparation of 1-(4-(4-(4-(methylamino)piperidin-1-yl)-6-((2-(thiophen-2-yl)ethyl)amino)-1,3,5-triazin-2-yl)-1,4-diazepan-1-yl)ethan-1-one (GTC00711)

With reference to Scheme M-57, the target compound GTC00711 was obtained (yellow solid, 3.6 g). ¹H NMR (400 MHz, MeOD) δ 7.25 (d, J=3.6 Hz, 1H), 6.94 (d, J=4.4 Hz, 2H), 4.58 (s, 2H), 4.07-3.82 (m, 4H), 3.81-3.54 (m, 6H), 3.43 (d, J=8.4 Hz, 1H), 3.27-3.06 (m, 4H), 2.74 (s, 3H), 2.24 (s, 2H), 2.15 (t, J=7.2 Hz, 3H), 1.99-1.82 (m, 2H), 1.70-1.53 (m, 2H). LCMS (ESI) calcd. 459.26; found, 459.4.

### Example 1 of Compound of Formula (II): Preparation of 1-(2-(2,4-dioxotetrahydropyrimidin-1(2H)-yl)-1,3-dioxoisoindolin-5-yl)piperidine-4-carbaldehyde (GT-05580)

With reference to the method of Scheme II-1, the reaction of the fluorinated substrate 5-fluoroisobenzofuran-1,3-dione and the 1-aminodihydropyrimidine-2,4 (1H,3H)-dione acetate salt prepared in Step 3 afforded the target compound GT-05580 (light green solid, 3.4 g, yield 79.64%). ¹H NMR (400 MHz, DMSO) § 10.84 (s, 1H), 9.62 (s, 1H), 7.72 (d, J=8.6 Hz, 1H), 7.40 (d, J=1.7 Hz, 1H), 7.30 (d, J=8.7 Hz, 1H), 3.97 (d, J=13.3 Hz, 2H), 3.78 (t, J=6.8 Hz, 2H), 3.20 (t, J=10.9 Hz, 2H), 2.83 (t, J=6.7 Hz, 2H), 2.74-2.61 (m, 1H), 1.97-1.86 (m, 2H), 1.65-1.47 (m, 2H). LCMS (ESI) calcd, for C₁₈H₁₉N₄O₅⁺ [M+H]⁺: 371.14; found, 371.2.

### Example 2 of Compound of Formula (II): Preparation of 1-(2-(2,4-dioxotetrahydropyrimidin-1(2H)-yl)-1,3-dioxoisoindolin-4-yl)piperidine-4-carbaldehyde (GT-05590)

With reference to the method of Scheme II-1, the reaction of the fluorinated substrate 4-fluoroisobenzofuran-1,3-dione and the 1-aminodihydropyrimidine-2,4 (1H,3H)-dione acetate salt prepared in Step 3 afforded the target compound GT-05590 (red solid, 2.71 g, yield 84.64%). ¹H NMR (400 MHz, DMSO) § 10.84 (s, 1H), 9.67 (s, 1H), 7.91-7.65 (m, 1H), 7.40 (t, J=7.2 Hz, 2H), 3.89-3.71 (m, 2H), 3.70-3.53 (m, 2H), 3.06 (dd, J=9.6, 5.5 Hz, 2H), 2.83 (dd, J=10.1, 4.8 Hz, 2H), 2.56 (d, J=4.2 Hz, 1H), 2.08-1.96 (m, 2H), 1.72 (dd, J=20.0, 8.4 Hz, 2H). LCMS (ESI) calcd, for C₁₈H₁₉N₄O₅⁺ [M+H]⁺: 371.14; found, 371.2.

### Example 3 of Compound of Formula (II): Preparation of 1′-(2-(2,4-dioxotetrahydropyrimidin-1(2H)-yl)-1,3-dioxoisoindolin-5-yl)-[1,4′-bipiperidine]-4-carbaldehyde (GT-05595)

With reference to the method of Scheme II-2, the reaction of the fluorinated substrate 2-(2,4-dioxotetrahydropyrimidin-1(2H)-yl)-5-fluoroisoindoline-1,3-dione and the 4-(dimethoxymethyl)-[1,4′-bipiperidine]prepared in Step 2 afforded the target compound GT-05595 as a hydrate (yellow solid, 1.0 g, yield 36.72%). ¹H NMR (400 MHz, DMSO) δ 10.83 (s, 1H), 9.57 (s, 1H), 7.70 (d, J=8.6 Hz, 1H), 7.37 (d, J=2.1 Hz, 1H), 7.28 (dd, J=8.7, 2.3 Hz, 1H), 4.10 (d, J=13.2 Hz, 2H), 3.77 (t, J=6.8 Hz, 2H), 2.97 (t, J=11.7 Hz, 2H), 2.89-2.80 (m, 2H), 2.77 (d, J=11.1 Hz, 2H), 2.58 (d, J=11.3 Hz, 1H), 2.25 (d, J=10.2 Hz, 2H), 2.03 (d, J=32.2 Hz, 1H), 1.85-1.69 (m, 4H), 1.58-1.34 (m, 4H). LCMS (ESI) calcd, for C₂₃H₃₀N₅O₆⁺ [M+H+H₂O]⁺: 472.22; found, 472.3.

### Example 4 of Compound of Formula (II): Preparation of 2-(2,4-dioxotetrahydropyrimidin-1(2H)-yl)-5-(piperazin-1-yl)isoindoline-1,3-dione (GT-05581)

With reference to the method of Scheme II-3, the reaction of the fluorinated substrate 2-(2,4-dioxotetrahydropyrimidin-1(2H)-yl)-5-fluoroisoindoline-1,3-dione and tert-butyl piperazine-1-carboxylate afforded the target compound GT-05581 (yellow solid, 3.22 g, yield 78.05%). ¹H NMR (400 MHz, MeOD) δ 7.81 (d, J=8.5 Hz, 1H), 7.52 (d, J=2.2 Hz, 1H), 7.39 (dd, J=8.5, 2.3 Hz, 1H), 3.86 (t, J=6.8 Hz, 2H), 3.77-3.68 (m, 4H), 3.46-3.36 (m, 4H), 2.94 (t, J=6.8 Hz, 2H). LCMS (ESI) calcd, for C₁₆H₁₈N₅O₄⁺ [M+H]⁺: 344.14; found, 344.2.

### Example 5 of Compound of Formula (II): Preparation of 2-(2,4-dioxotetrahydropyrimidin-1(2H)-yl)-5-(4-(piperazin-1-yl)piperidin-1-yl)isoindoline-1,3-dione (GT-05594)

With reference to the method of Scheme II-3, the reaction of the fluorinated substrate 2-(2,4-dioxotetrahydropyrimidin-1(2H)-yl)-5-fluoroisoindoline-1,3-dione and tert-butyl 4-(piperidin-4-yl)piperazine-1-carboxylate afforded the target compound GT-05594 (yellow solid, 9.12 g). ¹H NMR (400 MHz, MeOD) δ 7.74 (d, J=8.6 Hz, 1H), 7.44 (d, J=2.2 Hz, 1H), 7.32 (dd, J=8.6, 2.3 Hz, 1H), 4.24 (d, J=13.6 Hz, 2H), 3.85 (t, J=6.8 Hz, 2H), 3.57 (s, 8H), 3.53 (d, J=3.8 Hz, 1H), 3.07 (t, J=12.3 Hz, 2H), 2.93 (t, J=6.8 Hz, 2H), 2.23 (d, J=12.1 Hz, 2H), 1.82 (dd, J=12.1, 3.7 Hz, 2H). LCMS (ESI) calcd, for C₂₁H₂₇N₆O₄⁺ [M+H]⁺: 427.21; found, 427.3.

### Example 6 of Compound of Formula (II): Preparation of 5-(3,3-difluoro-4-(piperazin-1-yl)piperidin-1-yl)-2-(2,4-dioxotetrahydropyrimidin-1(2H)-yl)isoindoline-1,3-dione (GT-04990)

With reference to the method of Scheme II-3, the reaction of the fluorinated substrate 2-(2,4-dioxotetrahydropyrimidin-1(2H)-yl)-5-fluoroisoindoline-1,3-dione and benzyl 4-(3,3-difluoropiperidin-4-yl)piperazine-1-carboxylate hydrochloride afforded the target compound GT-04990 (yellow solid, 1.65 g). ¹H NMR (400 MHz, MeOD) δ 7.74 (d, J=8.6 Hz, 1H), 7.45 (s, 1H), 7.33 (d, J=8.7 Hz, 1H), 4.38-4.13 (m, 2H), 3.84 (t, J=6.8 Hz, 2H), 3.27-3.16 (m, 2H), 3.13 (d, J=3.6 Hz, 5H), 3.10-3.03 (m, 4H), 2.93 (t, J=6.8 Hz, 2H), 2.14-1.95 (m, 2H), 1.92 (s, 3H). LCMS (ESI) calcd, for C₂₁H₂₅F₂N₆₀₄⁺ [M+H]⁺: 463.19; found, 463.2.

### Example 7 of Compound of Formula (II): Preparation of 5-(bromomethyl)-2-(2,4-dioxotetrahydropyrimidin-1(2H)-yl)isoindoline-1,3-dione (GT-06979)

According to the method of Scheme II-4, the target compound 5-(bromomethyl)-2-(2,4-dioxotetrahydropyrimidin-1(2H)-yl)isoindoline-1,3-dione was obtained (yellow solid, 7.07 g, yield 96.79%). ¹H NMR (400 MHz, DMSO) δ 10.90 (s, 1H), 8.08 (s, 1H), 7.99 (s, 2H), 4.90 (s, 2H), 3.81 (t, J=6.8 Hz, 2H), 2.84 (t, J=6.8 Hz, 2H). LCMS (ESI) calcd, for C₁₃H₁₁BrN₃O₄⁺ [M+H]⁺: 351.99/353.99; found, 352.0/354.0.

### Example 8 of Compound of Formula (II): Preparation of 1-(5-(bromomethyl)-1-oxoisoindolin-2-yl)dihydropyrimidine-2,4 (1H,3H)-dione (GT-05568)

According to the method of Scheme II-5, the target compound 1-(5-(bromomethyl)-1-oxoisoindolin-2-yl)dihydropyrimidine-2,4 (1H,3H)-dione was obtained (yellow solid, 7.07 g, yield 96.79%). ¹H NMR (400 MHz, DMSO) δ 10.67 (s, 1H), 7.76 (d, J=7.8 Hz, 1H), 7.71 (s, 1H), 7.62 (d, J=7.9 Hz, 1H), 4.83 (s, 2H), 4.76 (d, J=16.6 Hz, 1H), 4.53 (d, J=16.3 Hz, 1H), 3.76 (d, J=5.2 Hz, 2H), 2.93-2.81 (m, 1H), 2.76 (s, 1H). LCMS (ESI) calcd, for C₁₃H₁₃BrN₃O₃⁺ [M+H]⁺: 338.01/340.01; found, 338.0/340.0.

### Example 9 of Compound of Formula (II): Preparation of 2-(2,4-dioxotetrahydropyrimidin-1(2H)-yl)-4-fluoro-5-(piperazin-1-yl)isoindoline-1,3-dione (GT-05592)

According to the method of Scheme II-6, the target compound 2-(2,4-dioxotetrahydropyrimidin-1(2H)-yl)-4-fluoro-5-(piperazin-1-yl)isoindoline-1,3-dione was obtained (yellow solid, 2.46 g). ¹H NMR (400 MHz, MeOD) δ 7.72 (d, J=8.1 Hz, 1H), 7.50 (t, J=7.8 Hz, 1H), 3.86 (t, J=6.8 Hz, 2H), 3.53 (dd, J=5.7, 3.7 Hz, 4H), 3.46-3.40 (m, 4H), 2.94 (t, J=6.8 Hz, 2H). LCMS (ESI) calcd, for C₁₈H₂₀FN₆O₄⁺ [M+H+CH₃CN]⁺: 403.15; found, 403.3.

### Example 10 of Compound of Formula (II): Preparation of 2-(2,4-dioxotetrahydropyrimidin-1(2H)-yl)-5-fluoro-6-(piperazin-1-yl)isoindoline-1,3-dione (GT-05584)

According to the method of Scheme II-3, the target compound 2-(2,4-dioxotetrahydropyrimidin-1(2H)-yl)-5-fluoro-6-(piperazin-1-yl)isoindoline-1,3-dione was obtained (light yellow solid, 2.36 g). ¹H NMR (400 MHz, MeOD) δ 7.69 (d, J=11.0 Hz, 1H), 7.64 (d, J=7.3 Hz, 1H), 3.86 (t, J=6.8 Hz, 2H), 3.65-3.50 (m, 4H), 3.48-3.38 (m, 4H), 2.94 (t, J=6.8 Hz, 2H). LCMS (ESI) calcd, for C₁₆H₁₇FN₅O₄⁺ [M+H]⁺: 362.13; found, 362.2.

### Example 11 of Compound of Formula (II): Preparation of 2-(2,4-dioxotetrahydropyrimidin-1(2H)-yl)-4-fluoro-6-(piperazin-1-yl)isoindoline-1,3-dione (GT-05593)

According to the method of Scheme II-6, the target compound 2-(2,4-dioxotetrahydropyrimidin-1(2H)-yl)-4-fluoro-6-(piperazin-1-yl)isoindoline-1,3-dione was obtained (yellow solid, 2.18 g). ¹H NMR (400 MHz, MeOD) δ 7.38 (d, J=2.1 Hz, 1H), 7.08 (dd, J=12.6, 2.0 Hz, 1H), 3.85 (td, J=6.9, 2.6 Hz, 2H), 3.79-3.69 (m, 4H), 3.44-3.34 (m, 4H), 2.93 (t, J=6.8 Hz, 2H). LCMS (ESI) calcd, for C₁₈H₂₀FN₆O₄⁺ [M+H+CH₃CN]⁺: 403.15; found, 403.2.

### Example 12 of Compound of Formula (II): Preparation of 2-(2,4-dioxotetrahydropyrimidin-1(2H)-yl)-4-(piperazin-1-yl)isoindoline-1,3-dione (GT-05591)

According to the method of Scheme II-3, the target compound 2-(2,4-dioxotetrahydropyrimidin-1(2H)-yl)-4-(piperazin-1-yl)isoindoline-1,3-dione was obtained (yellow solid, 3.46 g). ¹H NMR (400 MHz, MeOD) δ 7.80 (dd, J=8.4, 7.3 Hz, 1H), 7.55 (dd, J=7.2, 0.6 Hz, 1H), 7.44 (d, J=7.9 Hz, 1H), 3.92-3.81 (m, 2H), 3.67-3.56 (m, 4H), 3.50-3.42 (m, 4H), 3.02-2.85 (m, 2H). LCMS (ESI) calcd, for C₁₆H₁₈N₅O₄⁺ [M+H]⁺: 344.14; found, 344.3.

### Example 13 of Compound of Formula (II): Preparation of 5-((1R,4R)-2,5-(diazabicyclo [2.2.1]heptan-2-yl)-2-(2,4-dioxotetrahydropyrimidin-1(2H)-yl)isoindoline-1,3-dione (GT-05583)

According to the method of Scheme II-3, the target compound 5-((1R,4R)-2,5-diazabicyclo[2.2.1]heptan-2-yl)-2-(2,4-dioxotetrahydropyrimidin-1(2H)-yl)isoindoline-1,3-dione was obtained (yellow solid, 3.23 g). ¹H NMR (400 MHz, MeOD) δ 7.76 (d, J=8.4 Hz, 1H), 7.19 (dd, J=6.1, 2.0 Hz, 1H), 7.08-6.95 (m, 1H), 4.94 (s, 1H), 4.60 (s, 1H), 3.86 (d, J=7.0 Hz, 2H), 3.84-3.79 (m, 1H), 3.52 (d, J=11.1 Hz, 1H), 3.42 (q, J=11.4 Hz, 2H), 2.94 (t, J=6.8 Hz, 2H), 2.34 (d, J=11.3 Hz, 1H), 2.14 (d, J=11.3 Hz, 1H). LCMS (ESI) calcd, for C₁₇H₁₈N₅O₄⁺ [M+H]⁺: 356.14; found, 356.2.

### Example 14 of Compound of Formula (II): Preparation of 5-((1S,4S)-2,5-diazabicyclo[2.2.1]heptan-2-yl)-2-(2,4-dioxotetrahydropyrimidin-1(2H)-yl)isoindoline-1,3-dione (GT-05582)

According to the method of Scheme II-3, the target compound 5-((1S,4S)-2,5-diazabicyclo[2.2.1]heptan-2-yl)-2-(2,4-dioxotetrahydropyrimidin-1(2H)-yl)isoindoline-1,3-dione was obtained (yellow solid, 4.58 g). ¹H NMR (400 MHz, MeOD) δ 7.77 (d, J=8.4 Hz, 1H), 7.20 (dd, J=7.4, 2.2 Hz, 1H), 7.03 (ddd, J=8.4, 6.1, 2.3 Hz, 1H), 4.94 (s, 1H), 4.60 (s, 1H), 3.86 (t, J=3.9 Hz, 2H), 3.84-3.81 (m, 1H), 3.52 (d, J=11.0 Hz, 1H), 3.42 (q, J=11.5 Hz, 2H), 2.94 (t, J=6.8 Hz, 2H), 2.34 (d, J=11.4 Hz, 1H), 2.13 (d, J=11.3 Hz, 1H). LCMS (ESI) calcd, for C₁₇H₁₈N₅O₄⁺ [M+H]⁺: 356.14; found, 356.2.

### Example 15 of Compound of Formula (II): Preparation of 5-(3,6-diazabicyclo[3.1.1]heptan-6-yl)-2-(2,4-dioxotetrahydropyrimidin-1(2H)-yl)isoindoline-1,3-dione (GT-05588)

According to the method of Scheme II-3, the target compound 5-(3,6-diazabicyclo[3.1.1]heptan-6-yl)-2-(2,4-dioxotetrahydropyrimidin-1(2H)-yl)isoindoline-1,3-dione was obtained (yellow solid, 2.52 g). ¹H NMR (400 MHz, MeOD) δ 7.82 (d, J=8.3 Hz, 1H), 7.18 (d, J=2.0 Hz, 1H), 7.01 (dd, J=8.3, 2.1 Hz, 1H), 4.63 (d, J=6.3 Hz, 2H), 3.86 (t, J=6.9 Hz, 2H), 3.73 (d, J=13.1 Hz, 2H), 3.43 (d, J=12.2 Hz, 2H), 3.12-3.01 (m, 1H), 2.94 (t, J=6.8 Hz, 2H), 2.03 (d, J=10.0 Hz, 1H). LCMS (ESI) calcd, for C₁₉H₂₁N₆O₄⁺[M+H+CH₃CN]⁺: 397.16; found, 397.3.

### Example 16 of Compound of Formula (II): Preparation of 5-(3,6-diazabicyclo[3.1.1]heptan-3-yl)-2-(2,4-dioxotetrahydropyrimidin-1(2H)-yl)isoindoline-1,3-dione (GT-05587)

According to the method of Scheme II-3, the target compound 5-(3,6-diazabicyclo[3.1.1]heptan-3-yl)-2-(2,4-dioxotetrahydropyrimidin-1(2H)-yl)isoindoline-1,3-dione was obtained (yellow solid, 1.83 g). ¹H NMR (400 MHz, MeOD) δ 7.84 (d, J=8.5 Hz, 1H), 7.35 (d, J=2.2 Hz, 1H), 7.22 (dd, J=8.6, 2.3 Hz, 1H), 4.61 (d, J=6.4 Hz, 2H), 4.02 (dd, J=35.3, 12.4 Hz, 4H), 3.87 (t, J=6.8 Hz, 2H), 3.16-3.06 (m, 1H), 2.94 (t, J=6.8 Hz, 2H), 2.03 (d, J=10.7 Hz, 1H). LCMS (ESI) calcd, for C₁₇H₁₈N₅O₄⁺ [M+H]⁺: 356.14; found, 356.1.

### Example 17 Of Compound of Formula (II): Preparation of 5-(3,8-Diazabicyclo[3.2.1]Octan-8-Yl)-2-(2,4-dioxotetrahydropyrimidin-1(2H)-yl)isoindoline-1,3-dione (GT-05586)

According to the method of Scheme II-3, the target compound 5-(3,8-diazabicyclo[3.2.1]octan-8-yl)-2-(2,4-dioxotetrahydropyrimidin-1(2H)-yl)isoindoline-1,3-dione was obtained (yellow solid, 2.73 g). ¹H NMR (400 MHz, MeOD) δ 7.80 (d, J=8.5 Hz, 1H), 7.43 (d, J=2.2 Hz, 1H), 7.29 (dd, J=8.5, 2.3 Hz, 1H), 4.72 (s, 2H), 3.86 (t, J=6.8 Hz, 2H), 3.37-3.31 (m, 2H), 3.21 (d, J=12.9 Hz, 2H), 2.94 (t, J=6.8 Hz, 2H), 2.35 (dd, J=9.0, 4.5 Hz, 2H), 2.15 (t, J=7.4 Hz, 2H). LCMS (ESI) calcd, for C₁₈H₂₀N₅O₄⁺ [M+H]⁺: 370.15; found, 370.2.

### Example 18 Of Compound of Formula (II): Preparation of 5-(3,8-Diazabicyclo[3.2.1]Octan-3-Yl)-2-(2,4-dioxotetrahydropyrimidin-1(2H)-yl)isoindoline-1,3-dione (GT-05589)

According to the method of Scheme II-3, the target compound 5-(3,8-diazabicyclo[3.2.1]octan-3-yl)-2-(2,4-dioxotetrahydropyrimidin-1(2H)-yl)isoindoline-1,3-dione was obtained (yellow solid, 2.27 g). ¹H NMR (400 MHz, MeOD) δ 7.80 (d, J=8.5 Hz, 1H), 7.47 (d, J=2.2 Hz, 1H), 7.33 (dd, J=8.6, 2.3 Hz, 1H), 4.25 (s, 2H), 4.01 (d, J=11.6 Hz, 2H), 3.86 (t, J=6.8 Hz, 2H), 3.33 (s, 2H), 2.94 (t, J=6.8 Hz, 2H), 2.21-2.07 (m, 4H). LCMS (ESI) calcd, for C₁₈H₂₀N₅O₄⁺ [M+H]⁺: 370.15; found, 370.1.

### Example 19 Of Compound of Formula (II): Preparation of 5-(2,5-Diazabicyclo[2.2.2]Octan-2-Yl)-2-(2,4-dioxotetrahydropyrimidin-1(2H)-yl)isoindoline-1,3-dione (GT-05585)

According to the method of Scheme II-3, the target compound 5-(2,5-diazabicyclo[2.2.2]octan-2-yl)-2-(2,4-dioxotetrahydropyrimidin-1(2H)-yl)isoindoline-1,3-dione was obtained (yellow solid, 1.43 g). ¹H NMR (400 MHz, MeOD) δ 7.78 (dd, J=8.6, 1.0 Hz, 1H), 7.28 (dd, J=5.9, 2.3 Hz, 1H), 7.15-7.08 (m, 1H), 4.51 (s, 1H), 3.95 (s, 1H), 3.88 (d, J=8.7 Hz, 1H), 3.84 (d, J=6.8 Hz, 2H), 3.71 (dd, J=11.9, 1.8 Hz, 1H), 3.52 (t, J=10.9 Hz, 2H), 2.94 (t, J=6.8 Hz, 2H), 2.26-2.13 (m, 2H), 2.11-1.98 (m, 2H). LCMS (ESI) calcd. for C₂₀H₂₃N₆O₄⁺ [M+H+CH₃CN]⁺: 411.18; found, 411.2.

### Example 20 Of Compound of Formula (II): Preparation of 5-Bromo-2-(2,4-dioxotetrahydropyrimidin-1(2H)-yl)isoindoline-1,3-dione (GT-04511)

According to the method of Steps 1~4 of Scheme II-4, the target compound 5-bromo-2-(2,4-dioxotetrahydropyrimidin-1(2H)-yl)isoindoline-1,3-dione was obtained (white solid, 7.1 g, yield 95.34%). ¹H NMR (400 MHz, DMSO) δ 10.92 (s, 1H), 8.24 (d, J=1.4 Hz, 1H), 8.16 (dd, J=8.0, 1.7 Hz, 1H), 7.93 (d, J=8.0 Hz, 1H), 3.81 (t, J=6.8 Hz, 2H), 2.85 (t, J=6.8 Hz, 2H). LCMS (ESI) calcd, for C₁₂H₉BrN₃O₄⁺ [M+H]⁺: 337.97; found, 338.0.

### Example 21 of Compound of Formula (II): Preparation of 2-(2,4-dioxotetrahydropyrimidin-1(2H)-yl)-5-(hydroxymethyl)isoindoline-1,3-dione (GT-04536)

According to the method of Steps 1-5 of Scheme II-4, the target compound 2-(2,4-dioxotetrahydropyrimidin-1(2H)-yl)-5-(hydroxymethyl)isoindoline-1,3-dione was obtained (brown solid, 6.0 g, yield 90.15%). ¹H NMR (400 MHz, DMSO) δ 10.90 (s, 1H), 7.96 (d, J=7.7 Hz, 1H), 7.92-7.59 (m, 2H), 5.61 (d, J=5.7 Hz, 1H), 4.71 (d, J=5.5 Hz, 2H), 3.82 (t, J=6.8 Hz, 2H), 2.85 (t, J=6.8 Hz, 2H). LCMS (ESI) calcd, for C₁₃H₁₂N₃O₅⁺ [M+H]⁺: 290.08, found, 290.1.

### Example 22 Of Compound of Formula (II): Preparation of 1-(5-Bromo-1-Oxoisoindolin-2-yl)dihydropyrimidine-2,4 (1H,3H)-dione (GT-08972)

According to the method of Steps 1~4 of Scheme II-4, the target compound 1-(5-bromo-1-oxoisoindolin-2-yl)dihydropyrimidine-2,4 (1H,3H)-dione was obtained (white solid, 5.2 g, yield 58.91%). LCMS (ESI) calcd. for C₁₂H₁₁BrN₃O₃⁺ [M+H]⁺: 324.00, found, 324.0.

### Example 23 of Compound of Formula (II): Preparation of 1-(5-(hydroxymethyl)-1-oxoisoindolin-2-yl)dihydropyrimidine-2,4 (1H,3H)-dione (GT-05567)

According to the method of Steps 1~4 of Scheme II-5, the target compound 1-(5-(hydroxymethyl)-1-oxoisoindolin-2-yl)dihydropyrimidine-2,4 (1H,3H)-dione was obtained (white solid, 3.62 g, yield 78.79%). ¹H NMR (400 MHz, DMSO) δ 10.64 (s, 1H), 7.71 (d, J=7.8 Hz, 1H), 7.57 (s, 1H), 7.47 (d, J=7.9 Hz, 1H), 5.42 (t, J=5.7 Hz, 1H), 4.74 (d, J=16.1 Hz, 1H), 4.63 (d, J=5.7 Hz, 2H), 4.52 (d, J=16.3 Hz, 1H), 3.78 (dd, J=11.0, 6.5 Hz, 2H), 3.57 (s, 2H), 2.91-2.80 (m, 1H), 2.75 (d, J=5.5 Hz, 1H). LCMS (ESI) calcd. for C₁₃H₁₄N₃O₄⁺ [M+H]⁺: 276.09; found, 276.2.

### Example 24 of Compound of Formula (II): Preparation of 1-(4-hydroxy-1-oxoisoindolin-2-yl)dihydropyrimidine-2,4 (1H,3H)-dione (GT-05570)

According to the method of Scheme II-7, the target compound 1-(4-hydroxy-1-oxoisoindolin-2-yl)dihydropyrimidine-2,4 (1H,3H)-dione was obtained (white solid, 190 mg, yield 29.64%). ¹H NMR (400 MHz, DMSO) § 10.66 (s, 1H), 10.24 (s, 1H), 7.37 (t, J=7.8 Hz, 1H), 7.27-7.20 (m, 1H), 7.09 (dd, J=8.0, 0.7 Hz, 1H), 4.65 (d, J=16.3 Hz, 1H), 4.37 (d, J=16.2 Hz, 1H), 3.78 (t, J=6.8 Hz, 2H), 2.86 (dd, J=16.2, 8.3 Hz, 1H), 2.75 (dd, J=13.8, 8.2 Hz, 1H). LCMS (ESI) calcd, for C₁₂H₁₂N₃O₄⁺ [M+H]⁺: 262.07; found, 262.1.

### Example 25 of Compound of Formula (II): Preparation of 2-(2,4-dioxotetrahydropyrimidin-1(2H)-yl)-5-fluoroisoindoline-1,3-dione (GT-05575)

According to the method of Steps 1~4 of Scheme II-1, the target compound 2-(2,4-dioxotetrahydropyrimidin-1(2H)-yl)-5-fluoroisoindoline-1,3-dione was obtained (white solid, 13.2 g, yield 61.00%). ¹H NMR (400 MHz, DMSO) δ 10.95 (s, 1H), 8.09 (dd, J=8.3, 4.6 Hz, 1H), 7.95 (dd, J=7.4, 2.2 Hz, 1H), 7.79 (td, J=9.5, 2.3 Hz, 1H), 3.81 (t, J=6.8 Hz, 2H), 2.85 (t, J=6.8 Hz, 2H). LCMS (ESI) calcd. for C₁₂H₉FN₃O₄⁺ [M+H]⁺: 278.06; found, 278.1.

### Example 26 of Compound of Formula (II): Preparation of 2-(2,4-dioxotetrahydropyrimidin-1(2H)-yl)-4-fluoroisoindoline-1,3-dione (GT-05576)

According to the method of Steps 1~4 of Scheme II-1, the target compound 2-(2,4-dioxotetrahydropyrimidin-1(2H)-yl)-4-fluoroisoindoline-1,3-dione was obtained (white solid, 5.5 g, yield 51.24%). ¹H NMR (400 MHz, CDCl₃) δ 7.88-7.80 (m, 1H), 7.77 (d, J=7.3 Hz, 1H), 7.57 (s, 1H), 7.49 (t, J=8.5 Hz, 1H), 3.90 (td, J=6.9, 4.0 Hz, 2H), 3.03 (t, J=6.8 Hz, 2H). LCMS (ESI) calcd, for C₁₂H₉FN₃O₄⁺ [M+H]⁺: 278.06; found, 278.1.

### Example 27 of Compound of Formula (II): Preparation of 5-(aminomethyl)-2-(2,4-dioxotetrahydropyrimidin-1(2H)-yl)isoindoline-1,3-dione (GT-07968)

According to the method of Scheme II-8, the target compound 5-(aminomethyl)-2-(2,4-dioxotetrahydropyrimidin-1(2H)-yl)isoindoline-1,3-dione was obtained (white solid, 0.89 g). ¹H NMR (400 MHz, DMSO-d₆) δ 10.92 (s, 1H), 8.44 (s, 3H), 8.12 (s, 1H), 8.07 (d, J=7.7 Hz, 1H), 7.99 (d, J=7.8 Hz, 1H), 4.29 (s, 2H), 3.83 (t, J=6.8 Hz, 2H), 2.85 (t, J=6.8 Hz, 2H). LCMS (ESI) calcd, for C₁₃H₁₃N₄O₄⁺ [M+H]⁺: 289.09; found, 289.0.

### Example 28 of Compound of Formula (II): Preparation of 1-(5-(aminomethyl)-1-oxoisoindolin-2-yl)dihydropyrimidine-2,4 (1H,3H)-dione (GT-08118)

According to the method of Scheme II-9, the target compound 1-(5-(aminomethyl)-1-oxoisoindolin-2-yl)dihydropyrimidine-2,4 (1H,3H)-dione was obtained (white solid, 25 mg). ¹H NMR (400 MHz, DMSO-d₆) δ 10.67 (s, 1H), 8.25 (s, 3H), 7.84 (d, J=7.8 Hz, 1H), 7.68 (s, 1H), 7.61 (d, J=7.9 Hz, 1H), 4.79 (d, J=17.0 Hz, 1H), 4.54 (d, J=16.6 Hz, 1H), 4.18 (q, J=5.6 Hz, 2H), 3.78 (s, 2H), 2.85 (d, J=7.5 Hz, 1H), 2.76 (s, 1H). LCMS (ESI) calcd, for C₁₃H₁₅N₄O₃⁺ [M+H]⁺: 275.11; found, 275.2.

### Example 29 Of Compound of Formula (II): Preparation of 1-(1-Oxoisoindolin-2-yl)dihydropyrimidine-2,4 (1H,3H)-dione (GT-07014)

According to the method of Steps 1-3 of Scheme II-5, or the method of Scheme II-7, the target compound was obtained (white solid, 3.0 g). ¹H NMR (400 MHz, DMSO-d₆) δ 10.66 (s, 1H), 7.77 (d, J=7.6 Hz, 1H), 7.70 (td, J=7.5, 1.1 Hz, 1H), 7.64 (d, J=7.6 Hz, 1H), 7.55 (t, J=7.4 Hz, 1H), 4.76 (d, J=16.4 Hz, 1H), 4.54 (d, J=16.3 Hz, 1H), 3.88-3.71 (m, 2H), 2.86 (d, J=7.8 Hz, 1H), 2.76 (t, J=5.5 Hz, 1H). LCMS (ESI) calcd. for C₁₂H₁₂N₃O₃⁺: 246.09; found, 246.1.

### Example 1: Preparation of 2-(2-((cyclopropylmethyl)amino)pyridin-4-yl)-N-(3-(difluoromethyl)-1-(1-((2-(2,4-dioxotetrahydropyrimidin-1(2H)-yl)-1-oxoisoindolin-5-yl)methyl)piperidin-4-yl)-1H-pyrazol-4-yl) oxazole-4-carboxamide (GT-04951)

With reference to the method of Step 2 in Scheme T-1, the target compound (GT-04951) was obtained (white solid, 12 mg, yield 34.20%). ¹H NMR (400 MHz, DMSO) δ 10.95 (s, 1H), 10.69 (s, 1H), 9.92 (s, 1H), 9.10 (d, J=7.9 Hz, 1H), 8.21 (s, 1H), 8.09 (d, J=6.4 Hz, 1H), 7.90 (s, 2H), 7.80 (d, J=8.0 Hz, 1H), 7.55 (s, 1H), 7.24 (d, J=6.2 Hz, 1H), 4.83 (d, J=16.6 Hz, 1H), 4.60 (d, J=12.9 Hz, 2H), 4.49 (s, 2H), 3.79 (d, J=6.6 Hz, 2H), 3.51 (s, 2H), 3.16 (s, 4H), 2.89-2.74 (m, 2H), 2.37-2.25 (m, 4H), 1.19-1.07 (m, 1H), 0.55 (d, J=7.5 Hz, 2H), 0.31 (d, J=4.8 Hz, 2H). LCMS (ESI) calcd, for C₃₅H₃₇F₂N₁₀O₅⁺ [M+H]⁺: 715.29, found, 715.3.

### Example 2: Preparation of 2-(2-((cyclopropylmethyl)amino)pyridin-4-yl)-N-(3-(difluoromethyl)-1-(1-((2-(2,4-dioxotetrahydropyrimidin-1(2H)-yl)-1,3-dioxoisoindolin-5-yl)methyl)piperidin-4-yl)-1H-pyrazol-4-yl) oxazole-4-carboxamide (GT-04668)

With reference to the method of Step 2 in Scheme T-1, the target compound (GT-04668) was obtained (white solid, 10 mg, yield 23.80%). ¹H NMR (400 MHz, DMSO) δ 10.92 (s, 1H), 9.89 (s, 1H), 9.06 (s, 1H), 8.22 (s, 1H), 8.12 (dd, J=19.0, 6.5 Hz, 2H), 7.21 (s, 1H), 7.07 (s, 1H), 4.59 (s, 3H), 4.32 (s, 1H), 3.83 (t, J=6.7 Hz, 2H), 3.69 (d, J=6.5 Hz, 2H), 3.53 (s, 2H), 3.18 (s, 4H), 2.87 (d, J=6.7 Hz, 1H), 2.28 (s, 2H), 2.00 (d, J=8.0 Hz, 1H), 1.13 (d, J=7.3 Hz, 1H), 0.54 (d, J=7.8 Hz, 2H), 0.30 (d, J=4.5 Hz, 2H). LCMS (ESI) calcd. for C₃₅H₃₅F₂N₁₀O₆⁺ [M+H]⁺: 729.27, found, 729.3.

### Example 3: Preparation of 2-(2-((cyclopropylmethyl)amino)pyridin-4-yl)-N-(3-(difluoromethyl)-1-(1-((1-(2-(2,4-dioxotetrahydropyrimidin-1(2H)-yl)-1,3-dioxoisoindolin-5-yl)piperidin-4-yl)methyl)piperidin-4-yl)-1H-pyrazol-4-yl) oxazole-4-carboxamide (GT-04953)

With reference to the method of Scheme T-3, the target compound (GT-04953) was obtained (white solid, 28 mg, yield 38.30%). ¹H NMR (400 MHz, DMSO) δ 10.84 (s, 1H), 9.97 (d, J=11.9 Hz, 1H), 9.12 (s, 1H), 8.24 (s, 1H), 8.09 (d, J=6.4 Hz, 1H), 7.73 (d, J=8.6 Hz, 1H), 7.60 (s, 1H), 7.42 (s, 1H), 7.26 (ddd, J=63.4, 38.5, 27.1 Hz, 3H), 4.60 (s, 1H), 4.12 (s, 1H), 3.78 (t, J=6.9 Hz, 2H), 3.68 (d, J=10.0 Hz, 2H), 3.32 (d, J=6.9 Hz, 4H), 3.08 (dd, J=25.8, 11.7 Hz, 6H), 2.83 (t, J=7.0 Hz, 2H), 2.26 (d, J=12.6 Hz, 3H), 1.94 (d, J=11.2 Hz, 2H), 1.37-1.06 (m, 4H), 0.59-0.52 (m, 2H), 0.33 (q, J=4.8 Hz, 2H). LCMS (ESI) calcd. for C₄₀H₄₄F₂N₁₁O₆⁺ [M+H]⁺: 812.34, found, 812.4.

### Example 4: Preparation of 2-(2-((cyclopropylmethyl)amino)pyridin-4-yl)-N-(3-(difluoromethyl)-1-(1-((1-(2-(2,4-dioxotetrahydropyrimidin-1(2H)-yl)-1,3-dioxoisoindolin-4-yl)piperidin-4-yl)methyl)piperidin-4-yl)-1H-pyrazol-4-yl) oxazole-4-carboxamide (GT-05132)

With reference to the method of Scheme T-3, the target compound (GT-05132) was obtained (yellow solid, 12 mg, yield 53.92%). ¹H NMR (400 MHz, DMSO) δ 10.86 (s, 1H), 9.96 (s, 1H), 9.09 (s, 1H), 8.24 (s, 1H), 8.10 (d, J=6.2 Hz, 1H), 7.79-7.72 (m, 1H), 7.54 (s, 1H), 7.45-7.21 (m, 4H), 4.61 (s, 1H), 3.82-3.67 (m, 6H), 3.30 (d, J=7.2 Hz, 4H), 3.16-3.05 (m, 4H), 2.96-2.78 (m, 4H), 2.27 (d, J=12.0 Hz, 2H), 2.14-1.94 (m, 3H), 1.47 (s, 2H), 1.14 (s, 1H), 0.55 (d, J=7.6 Hz, 2H), 0.31 (d, J=5.1 Hz, 2H). LCMS (ESI) calcd. for C₄₀H₄₄F₂N₁₁O₆⁺ [M+H]⁺: 812.34, found, 812.3.

### Example 5: Preparation of 2-(2-((cyclopropylmethyl)amino)pyridin-4-yl)-N-(3-(difluoromethyl)-1-(1-((1′-(2-(2,4-dioxotetrahydropyrimidin-1(2H)-yl)-1,3-dioxoisoindolin-5-yl)-[1,4′-bipiperidin]-4-yl)methyl)piperidin-4-yl)-1H-pyrazol-4-yl) oxazole-4-carboxamide (GT-05133)

With reference to the method of Scheme T-3, the target compound (GT-05133) was obtained (yellow solid, 21 mg, yield 58.37%). ¹H NMR (400 MHz, DMSO) δ 10.85 (s, 1H), 10.62 (s, 1H), 10.32 (s, 1H), 9.94 (s, 1H), 9.07 (s, 1H), 8.23 (s, 1H), 8.10 (d, J=6.3 Hz, 1H), 7.76 (d, J=8.6 Hz, 1H), 7.47 (s, 2H), 7.38-7.06 (m, 3H), 4.59 (s, 1H), 4.28 (d, J=12.0 Hz, 2H), 3.79 (t, J=6.9 Hz, 2H), 3.68 (d, J=11.6 Hz, 2H), 3.49 (s, 4H), 3.25-3.22 (m, 2H), 3.14-2.92 (m, 8H), 2.83 (t, J=7.1 Hz, 2H), 2.54 (d, J=5.6 Hz, 2H), 2.31-2.04 (m, 7H), 1.71 (dd, J=25.2, 11.6 Hz, 4H), 1.13 (s, 1H), 0.54 (d, J=6.9 Hz, 2H), 0.30 (d, J=4.9 Hz, 2H). LCMS (ESI) calcd. for C₄₅H₅₃F₂N₁₂O₆⁺ [M+H]⁺: 895.42, found, 895.5.

### Example 6: Preparation of N-(3-(difluoromethyl)-1-(1-((1-(2-(2,4-dioxotetrahydropyrimidin-1(2H)-yl)-1,3-dioxoisoindolin-5-yl)piperidin-4-yl)methyl)piperidin-4-yl)-1H-pyrazol-4-yl)-2-(2-((2,2,2-trifluoroethyl)amino)pyridin-4-yl) oxazole-4-carboxamide (GT-05142)

With reference to the method of Scheme T-3, the target compound (GT-05142) was obtained (yellow solid, 29 mg, yield 72.13%). ¹H NMR (400 MHz, DMSO) δ 10.85 (s, 1H), 9.97 (s, 1H), 9.88 (d, J=13.3 Hz, 1H), 9.01 (s, 1H), 8.25 (d, J=4.9 Hz, 2H), 7.89 (s, 1H), 7.73 (dd, J=8.6, 2.5 Hz, 1H), 7.42 (d, J=2.2 Hz, 1H), 7.39-7.30 (m, 2H), 7.27-7.09 (m, 2H), 4.60 (s, 1H), 4.28 (dt, J=15.4, 7.6 Hz, 2H), 4.14 (d, J=13.3 Hz, 2H), 3.79 (t, J=6.8 Hz, 2H), 3.68 (d, J=11.9 Hz, 2H), 3.19-2.96 (m, 6H), 2.83 (t, J=7.2 Hz, 2H), 2.43 (d, J=13.0 Hz, 2H), 2.32-2.11 (m, 3H), 1.94 (d, J=11.5 Hz, 2H), 1.29 (d, J=11.8 Hz, 2H). LCMS (ESI) calcd. for C₃₈H₃₉F₅N₁₁O₆⁺ [M+H]⁺: 840.30, found, 840.3.

### Example 7: Preparation of N-(3-(difluoromethyl)-1-(1-((1-(2-(2,4-dioxotetrahydropyrimidin-1(2H)-yl)-1,3-dioxoisoindolin-4-yl)piperidin-4-yl)methyl)piperidin-4-yl)-1H-pyrazol-4-yl)-2-(2-((2,2,2-trifluoroethyl)amino)pyridin-4-yl) oxazole-4-carboxamide (GT-05143)

With reference to the method of Scheme T-3, the target compound (GT-05143) was obtained (yellow solid, 45 mg, yield 83.94%). ¹H NMR (400 MHz, DMSO) δ 10.86 (s, 1H), 10.02 (s, 1H), 9.90 (s, 1H), 9.01 (s, 1H), 8.30-8.17 (m, 2H), 7.92 (s, 1H), 7.74 (d, J=7.2 Hz, 1H), 7.41 (t, J=8.3 Hz, 2H), 7.23 (dd, J=47.2, 43.4 Hz, 3H), 4.62 (t, J=20.9 Hz, 1H), 4.30 (d, J=9.3 Hz, 2H), 3.87-3.64 (m, 6H), 3.10 (s, 4H), 2.83 (t, J=6.7 Hz, 4H), 2.47 (s, 2H), 2.29 (s, 2H), 1.99 (d, J=11.6 Hz, 3H), 1.58-1.39 (m, 2H). LCMS (ESI) calcd. for C₃₈H₃₉F₅N₁₁O₆⁺ [M+H]⁺: 840.30, found, 840.3.

### Example 8: Preparation of N-(3-(difluoromethyl)-1-(1-((1′-(2-(2,4-dioxotetrahydropyrimidin-1(2H)-yl)-1,3-dioxoisoindolin-5-yl)-[1,4′-bipiperidin]-4-yl)methyl)piperidin-4-yl)-1H-pyrazol-4-yl)-2-(2-((2,2,2-trifluoroethyl)amino)pyridin-4-yl) oxazole-4-carboxamide (GT-05144)

With reference to the method of Scheme T-3, the target compound (GT-05144) was obtained (white solid, 32 mg, yield 88.67%). ¹H NMR (400 MHz, DMSO) § 10.85 (s, 1H), 10.65 (s, 1H), 10.26 (s, 1H), 9.87 (d, J=7.9 Hz, 1H), 8.99 (s, 1H), 8.24 (d, J=5.2 Hz, 2H), 7.84-7.73 (m, 2H), 7.47 (s, 1H), 7.39-7.33 (m, 1H), 7.29 (s, 1H), 7.25-7.07 (m, 2H), 4.59 (s, 1H), 4.28 (d, J=10.1 Hz, 4H), 3.79 (t, J=6.8 Hz, 2H), 3.68 (d, J=11.3 Hz, 2H), 3.48 (s, 4H), 3.23 (s, 2H), 3.20-2.91 (m, 8H), 2.83 (t, J=6.9 Hz, 2H), 2.31-2.02 (m, 7H), 1.71 (dd, J=24.8, 12.1 Hz, 3H). LCMS (ESI) calcd, for C₄₃H₄₈F₅N₁₂O₆⁺ [M+H]⁺: 923.37, found, 923.4.

### Example 9: Preparation of N-(2-(1-((2-(2,4-dioxotetrahydropyrimidin-1(2H)-yl)-1-oxoisoindolin-5-yl)methyl)piperidin-4-yl)-6-methoxy-2H-indazol-5-yl)-3-(trifluoromethyl)benzamide (GT-04876)

With reference to the method of Step 2 in Scheme T-1, the target compound was obtained (GT-04876) (white solid, 44 mg, yield 68.12%). ¹H NMR (400 MHz, DMSO) δ 10.88 (s, 1H), 10.69 (s, 1H), 9.80 (d, J=17.2 Hz, 1H), 8.32-8.26 (m, 2H), 8.05 (d, J=6.2 Hz, 1H), 7.97 (d, J=7.5 Hz, 1H), 7.91 (d, J=7.4 Hz, 2H), 7.79 (dd,J=15.9, 8.0 Hz, 2H), 7.09 (s, 1H), 4.90-4.70 (m, 2H), 4.64-4.49 (m, 3H), 3.88 (d, J=7.7 Hz, 3H), 3.80 (t, J=6.7 Hz, 2H), 3.64-3.53 (m, 2H), 3.28-3.09 (m, 3H), 2.94-2.72 (m, 2H), 2.54 (d, J=5.5 Hz, 1H), 2.34 (d, J=14.9 Hz, 2H). LCMS (ESI) calcd, for C₃₄H₃₃F₃N₇O₅⁺ [M+H]⁺: 676.25, found, 676.3.

### Example 10: Preparation of N-(2-(1-((2-(2,4-dioxotetrahydropyrimidin-1(2H)-yl)-1,3-dioxoisoindolin-5-yl)methyl)piperidin-4-yl)-6-methoxy-2H-indazol-5-yl)-3-(trifluoromethyl)benzamide (GT-05078)

With reference to the method of Step 2 in Scheme T-1, the target compound (GT-05078) was obtained (white solid, 20 mg, yield 30.34%). ¹H NMR (400 MHz, DMSO) δ 11.07 (s, 1H), 10.94 (s, 1H), 9.81 (d, J=16.9 Hz, 1H), 8.31 (dd, J=19.1, 7.5 Hz, 3H), 8.26-8.10 (m, 2H), 8.07-8.01 (m, 1H), 7.97 (d, J=7.7 Hz, 1H), 7.78 (t, J=7.6 Hz, 1H), 7.10 (s, 1H), 4.68 (d, J=57.8 Hz, 3H), 3.94-3.79 (m, 5H), 3.57 (d, J=11.0 Hz, 2H), 3.21 (d, J=30.0 Hz, 3H), 2.87 (t, J=6.8 Hz, 2H), 2.48-2.42 (m, 1H), 2.34 (d, J=10.6 Hz, 2H). LCMS (ESI) calcd. for C₃₄H₃₁F₃N₇O₆⁺ [M+H]⁺: 690.23, found, 690.2.

### Example 11: Preparation of N-(2-(1-((1-(2-(2,4-dioxotetrahydropyrimidin-1(2H)-yl)-1,3-dioxoisoindolin-5-yl)piperidin-4-yl)methyl)piperidin-4-yl)-6-methoxy-2H-indazol-5-yl)-3-(trifluoromethyl)benzamide (GT-04877)

With reference to the method of Scheme T-3, the target compound (GT-04877) was obtained (white solid, 50 mg, yield 67.68%). ¹H NMR (400 MHz, DMSO) δ 10.84 (s, 1H), 10.09 (d, J=76.8 Hz, 1H), 9.85-9.59 (m, 1H), 8.41 (d, J=47.2 Hz, 1H), 8.33-8.25 (m, 2H), 8.06 (s, 1H), 7.97 (d, J=7.9 Hz, 1H), 7.83-7.66 (m, 2H), 7.43 (s, 1H), 7.33 (d, J=9.0 Hz, 1H), 7.10 (s, 1H), 4.76 (s, 1H), 4.14 (d, J=13.2 Hz, 2H), 3.88 (s, 3H), 3.79 (t, J=6.8 Hz, 2H), 3.72 (d, J=12.0 Hz, 2H), 3.44 (s, 2H), 3.18 (dd, J=23.2, 11.6 Hz, 3H), 3.05 (t, J=11.2 Hz, 3H), 2.83 (t, J=6.8 Hz, 2H), 2.58 (d, J=13.4 Hz, 2H), 2.35 (d, J=14.4 Hz, 2H), 2.23 (s, 1H), 1.95 (d, J=10.8 Hz, 2H). LCMS (ESI) calcd, for C₃₉H₄₀F₃N₈O₆⁺ [M+H]⁺: 773.30, found, 773.3.

### Example 12: Preparation of N-(2-(1-((2-(2,4-dioxotetrahydropyrimidin-1(2H)-yl)-1-oxoisoindolin-5-yl)methyl)piperidin-4-yl)-6-methoxy-2H-indazol-5-yl)-6-(trifluoromethyl) nicotinamide (GT-04880)

With reference to the method of Scheme T-3, the target compound (GT-04880) was obtained (white solid, 45 mg, yield 69.74%). ¹H NMR (400 MHz, DMSO) δ 10.96 (s, 1H), 10.69 (s, 1H), 10.52 (d, J=8.7 Hz, 1H), 8.72 (d, J=12.6 Hz, 1H), 8.55-8.38 (m, 2H), 8.35 (s, 1H), 8.23 (t, J=6.2 Hz, 1H), 7.97-7.80 (m, 3H), 7.18 (s, 1H), 4.80 (dt, J=22.3, 12.8 Hz, 2H), 4.61 (d, J=16.7 Hz, 2H), 3.99 (d, J=9.0 Hz, 3H), 3.80 (t, J=6.8 Hz, 2H), 3.60 (ddd, J=21.7, 10.8, 8.1 Hz, 2H), 3.14 (ddd, J=22.1, 17.8, 14.7 Hz, 3H), 2.92-2.84 (m, 1H), 2.76 (d, J=22.1 Hz, 1H), 2.54 (d, J=5.6 Hz, 1H), 2.48-2.42 (m, 1H), 2.34 (d, J=11.6 Hz, 2H). LCMS (ESI) calcd, for C₃₃H₃₂F₃N₈O₅⁺ [M+H]⁺: 677.24, found, 677.3.

### Example 13: Preparation of N-(2-(1-((2-(2,4-dioxotetrahydropyrimidin-1(2H)-yl)-1,3-dioxoisoindolin-5-yl)methyl)piperidin-4-yl)-6-methoxy-2H-indazol-5-yl)-6-(trifluoromethyl) nicotinamide (GT-05079)

With reference to the method of Scheme T-3, the target compound (GT-05079) was obtained (white solid, 25 mg, yield 37.95%). ¹H NMR (400 MHz, DMSO) δ 10.93 (s, 2H), 10.51 (s, 1H), 8.71 (s, 1H), 8.55-8.32 (m, 4H), 8.21 (dd, J=11.9, 7.6 Hz, 2H), 8.14 (d, J=7.7 Hz, 1H), 7.18 (s, 1H), 4.74 (s, 1H), 4.61 (s, 2H), 3.99 (d, J=9.2 Hz, 3H), 3.84 (t, J=6.8 Hz, 2H), 3.57 (d, J=10.9 Hz, 2H), 3.24 (s, 2H), 2.87 (t, J=6.9 Hz, 2H), 2.49-2.42 (m, 2H), 2.39-2.22 (m, 2H). LCMS (ESI) calcd, for C₃₃H₃₀F₃N₈O₆⁺ [M+H]⁺: 691.22, found, 691.3.

### Example 14: Preparation of N-(2-(1-((1-(2-(2,4-dioxotetrahydropyrimidin-1(2H)-yl)-1,3-dioxoisoindolin-5-yl)piperidin-4-yl)methyl)piperidin-4-yl)-6-methoxy-2H-indazol-5-yl)-6-(trifluoromethyl) nicotinamide (GT-04883)

With reference to the method of Scheme T-3, the target compound (GT-04883) was obtained (white solid, 20 mg, yield 27.10%). ¹H NMR (400 MHz, DMSO) δ 10.84 (s, 1H), 10.52 (s, 1H), 10.05 (s, 1H), 8.72 (s, 1H), 8.51-8.34 (m, 3H), 8.23 (d, J=7.7 Hz, 1H), 7.73 (dd, J=8.5, 4.0 Hz, 1H), 7.43 (s, 1H), 7.33 (d, J=7.0 Hz, 1H), 7.19 (s, 1H), 4.78 (dd, J=34.3, 22.7 Hz, 1H), 4.14 (d, J=13.3 Hz, 2H), 4.00 (s, 3H), 3.79 (t, J=6.7 Hz, 2H), 3.72 (d, J=12.3 Hz, 2H), 3.24-2.99 (m, 6H), 2.83 (t, J=6.9 Hz, 2H), 2.63-2.54 (m, 2H), 2.34 (d, J=11.5 Hz, 2H), 2.23 (s, 1H), 1.95 (d, J=11.6 Hz, 2H), 1.31 (d, J=9.3 Hz, 2H). LCMS (ESI) calcd. for C₃₈H₃₉F₃N₉O₆⁺ [M+H]⁺: 774.30, found, 774.3.

### Example 15: Preparation of N-(3-(difluoromethyl)-1-(1-((2-(2,4-dioxotetrahydropyrimidin-1(2H)-yl)-1,3-dioxoisoindolin-5-yl)methyl)piperidin-4-yl)-1H-pyrazol-4-yl)-5-morpholinopyrazolo[1,5-alpyrimidine-3-carboxamide (GT-04621)

With reference to the method of Scheme T-3, the target compound (GT-04621) was obtained (white solid, 26 mg, yield 53.91%). ¹H NMR (400 MHz, DMSO) δ 10.82 (d, J=29.4 Hz, 2H), 9.37 (d, J=13.8 Hz, 1H), 8.76 (d, J=7.9 Hz, 1H), 8.36 (s, 1H), 8.32-8.20 (m, 2H), 8.07 (t, J=7.3 Hz, 2H), 7.06 (t, J=53.6 Hz, 1H), 6.84 (d, J=7.9 Hz, 1H), 4.55 (d, J=32.8 Hz, 3H), 3.75 (dd, J=14.4, 7.5 Hz, 6H), 3.65 (d, J=4.0 Hz, 4H), 3.46 (s, 2H), 3.10 (s, 2H), 2.79 (t, J=6.7 Hz, 2H), 2.23 (d, J=23.9 Hz, 4H). LCMS (ESI) calcd, for C₃₃H₃₄F₂N₁₁O₆⁺[M+H]⁺: 718.27, found, 718.3.

### Example 16: Preparation of 5-((1R,4R)-2-oxa-5-azabicyclo[2.2.1]heptan-5-yl)-N-(3-(difluoromethyl)-1-(1-((2-(2,4-dioxotetrahydropyrimidin-1(2H)-yl)-1,3-dioxoisoindolin-5-yl)methyl)piperidin-4-yl)-1H-pyrazol-4-yl)pyrazolo[1,5-a]pyrimidine-3-carboxamide (GT-05080)

With reference to the method of Step 2 in Scheme T-1, the target compound was obtained (GT-05080) (white solid, 25 mg, yield 39.27%). ¹H NMR (400 MHz, DMSO) δ 11.03 (s, 1H), 10.92 (d, J=10.3 Hz, 1H), 9.52 (d, J=6.2 Hz, 1H), 8.78 (d, J=7.6 Hz, 1H), 8.43 (d, J=3.0 Hz, 1H), 8.34 (d, J=14.8 Hz, 1H), 8.27 (t, J=7.3 Hz, 1H), 8.15 (dd, J=22.7, 7.7 Hz, 2H), 8.05-7.91 (m, 1H), 7.15 (dd, J=55.2, 51.8 Hz, 1H), 6.66 (dd, J=164.9, 7.8 Hz, 1H), 5.17 (d, J=77.5 Hz, 1H), 4.77 (d, J=19.7 Hz, 1H), 4.57 (s, 3H), 3.82 (dd, J=6.9, 4.0 Hz, 3H), 3.59 (dd, J=33.1, 19.8 Hz, 5H), 3.17 (s, 2H), 2.87 (t, J=6.2 Hz, 2H), 2.30 (d, J=21.8 Hz, 4H), 2.00 (dd, J=30.7, 11.7 Hz, 2H). LCMS (ESI) calcd, for C₃₄H₃₄F₂N₁₁O₆⁺ [M+H]⁺: 730.27, found, 730.3.

### Example 17: Preparation of 1-(5-((4-(3-amino-6-(2-hydroxyphenyl)pyridazin-4-yl)piperazin-1-yl)methyl)-1-oxoisoindolin-2-yl)dihydropyrimidine-2,4 (1H,3H)-dione (GT-04952)

With reference to the method of Step 2 in Scheme T-1, the target compound (GT-04952) was obtained (white solid, 30 mg, yield 38.50%). ¹H NMR (400 MHz, DMSO) δ 12.05 (s, 1H), 10.69 (s, 1H), 7.95 (s, 1H), 7.86 (dd, J=20.4, 7.8 Hz, 2H), 7.61 (d, J=8.3 Hz, 2H), 7.39 (t, J=7.6 Hz, 1H), 7.08 (d, J=8.3 Hz, 1H), 6.98 (t, J=7.5 Hz, 1H), 4.82 (d, J=16.6 Hz, 1H), 4.65-4.44 (m, 3H), 3.79 (t, J=6.8 Hz, 4H), 3.60 (ddd, J=13.4, 10.6, 6.7 Hz, 4H), 3.20-3.07 (m, 2H), 2.91-2.73 (m, 2H). LCMS (ESI) calcd, for C₂₇H₂₉N₈O₄⁺ [M+H]⁺: 529.23, found, 529.3.

### Example 18: Preparation of 5-((4-(3-amino-6-(2-hydroxyphenyl)pyridazin-4-yl)piperazin-1-yl)methyl)-2-(2,4-dioxotetrahydropyrimidin-1(2H)-yl)isoindoline-1,3-dione (GT-05071)

With reference to the method of Step 2 in Scheme T-1, the target compound (GT-05071) was obtained (white solid, 27 mg, yield 33.75%). ¹H NMR (400 MHz, DMSO) δ 10.97-10.85 (m, 1H), 8.35 (s, 1H), 8.23 (d, J=7.8 Hz, 1H), 8.12 (d, J=7.7 Hz, 1H), 7.61 (t, J=8.8 Hz, 3H), 7.39 (t, J=7.4 Hz, 1H), 7.08 (d, J=8.1 Hz, 1H), 6.98 (t, J=7.5 Hz, 1H), 4.55 (d, J=40.6 Hz, 2H), 3.83 (t, J=6.8 Hz, 2H), 3.65 (dd, J=18.0, 11.3 Hz, 8H), 2.86 (t, J=6.8 Hz, 2H). LCMS (ESI) calcd, for C₂₇H₂₇N₈O₅⁺ [M+H]⁺: 543.21, found, 543.2.

### Example 19: Preparation of 5-(4-((4-(3-amino-6-(2-hydroxyphenyl)pyridazin-4-yl)piperazin-1-yl)methyl)piperidin-1-yl)-2-(2,4-dioxotetrahydropyrimidin-1(2H)-yl)isoindoline-1,3-dione (GT-04954)

With reference to the method of Scheme T-3, the target compound (GT-04954) was obtained (white solid, 35 mg, yield 37.94%). ¹H NMR (400 MHz, DMSO) δ 10.81 (d, J=23.5 Hz, 1H), 7.72 (d, J=8.6 Hz, 1H), 7.67-7.56 (m, 2H), 7.46-7.37 (m, 2H), 7.33 (d, J=8.7 Hz, 1H), 7.14 (d, J=8.2 Hz, 1H), 6.99 (t, J=7.5 Hz, 1H), 4.12 (d, J=12.7 Hz, 2H), 3.64 (d, J=11.1 Hz, 6H), 3.33 (d, J=8.7 Hz, 3H), 3.14-3.00 (m, 4H), 2.88-2.78 (m, 2H), 2.23 (s, 1H), 2.01 (d, J=10.8 Hz, 2H), 1.38-1.21 (m, 2H). LCMS (ESI) calcd, for C₃₂H₃₆N₉O₅⁺ [M+H]⁺: 626.28, found, 626.3.

### Example 20: Preparation of 4-(4-((4-(3-amino-6-(2-hydroxyphenyl)pyridazin-4-yl)piperazin-1-yl)methyl)piperidin-1-yl)-2-(2,4-dioxotetrahydropyrimidin-1(2H)-yl)isoindoline-1,3-dione (GT-05070)

With reference to the method of Scheme T-3, the target compound (GT-05070) was obtained (white solid, 30 mg, yield 32.52%). ¹H NMR (400 MHz, DMSO) δ 11.05 (s, 1H), 10.85 (s, 1H), 7.80-7.73 (m, 1H), 7.68-7.59 (m, 2H), 7.45-7.35 (m, 3H), 7.09 (d, J=8.1 Hz, 1H), 6.99 (t, J=7.4 Hz, 1H), 3.88-3.75 (m, 4H), 3.72-3.51 (m, 7H), 3.13 (s, 2H), 3.02-2.91 (m, 2H), 2.83 (dd, J=12.1, 6.4 Hz, 2H), 2.08 (d, J=29.6 Hz, 3H), 1.48 (s, 2H). LCMS (ESI) calcd, for C₃₂H₃₆N₉O₅⁺ [M+H]⁺: 626.28, found, 626.3.

### Example 21: Preparation of 5-(4-((4-(3-amino-6-(2-hydroxyphenyl)pyridazin-4-yl)piperazin-1-yl)methyl)-[1,4′-bipiperidin]-1′-yl)-2-(2,4-dioxotetrahydropyrimidin-1(2H)-yl)isoindoline-1,3-dione (GT-05095)

With reference to the method of Scheme T-3, the target compound (GT-05095) was obtained (yellow solid, 16 mg, yield 15.31%). ¹H NMR (400 MHz, DMSO) δ 11.37 (s, 1H), 10.92 (s, 1H), 10.84 (s, 1H), 7.75 (d, J=8.6 Hz, 1H), 7.60 (d, J=7.8 Hz, 2H), 7.46 (s, 1H), 7.40 (s, 2H), 7.12 (d, J=8.2 Hz, 1H), 6.99 (s, 1H), 4.28 (d, J=13.0 Hz, 2H), 3.79 (t, J=6.8 Hz, 4H), 3.63 (s, 4H), 3.50 (s, 4H), 3.02 (dd, J=29.0, 16.3 Hz, 6H), 2.83 (t, J=6.9 Hz, 2H), 2.24-2.07 (m, 5H), 1.74 (d, J=11.7 Hz, 4H). LCMS (ESI) calcd, for C₃₇H₄₅N₁₀O₅⁺ [M+H]⁺: 709.36, found, 709.4.

### Example 22: Preparation of 1-(5-((4-(3-amino-6-(5-fluoro-2-hydroxyphenyl)pyridazin-4-yl)piperazin-1-yl)methyl)-1-oxoisoindolin-2-yl)dihydropyrimidine-2,4 (1H,3H)-dione (GT-05061)

With reference to the method of Step 2 in Scheme T-1, the target compound (GT-05061) was obtained (white solid, 45 mg, yield 59.55%). ¹H NMR (400 MHz, DMSO) δ 12.03 (s, 1H), 10.70 (s, 1H), 7.96 (s, 1H), 7.87 (dd, J=20.1, 7.9 Hz, 2H), 7.66 (s, 1H), 7.55 (dd, J=9.4, 3.1 Hz, 1H), 7.24 (td, J=8.6, 3.2 Hz, 1H), 7.08 (dd, J=9.0, 4.8 Hz, 1H), 4.93-4.58 (m, 2H), 4.55 (d, J=12.5 Hz, 2H), 3.80 (t, J=6.8 Hz, 2H), 3.73 (s, 6H), 3.23-3.04 (m, 2H), 2.92-2.75 (m, 2H). LCMS (ESI) calcd, for C₂₇H₂₈FN₈O₄⁺ [M+H]⁺: 547.22, found, 547.3.

### Example 23: Preparation of 5-((4-(3-amino-6-(5-fluoro-2-hydroxyphenyl)pyridazin-4-yl)piperazin-1-yl)methyl)-2-(2,4-dioxotetrahydropyrimidin-1(2H)-yl)isoindoline-1,3-dione (GT-05072)

With reference to the method of Step 2 in Scheme T-1, the target compound (GT-05072) was obtained (white solid, 26 mg, yield 33.55%). ¹H NMR (400 MHz, DMSO) § 12.08 (s, 1H), 10.91 (d, J=14.0 Hz, 1H), 8.35 (s, 1H), 8.22 (d, J=7.8 Hz, 1H), 8.13 (d, J=7.7 Hz, 1H), 7.92 (d, J=14.8 Hz, 1H), 7.65 (s, 1H), 7.57 (dd, J=9.6, 3.0 Hz, 2H), 7.23 (d, J=2.8 Hz, 1H), 7.05 (dd, J=8.9, 4.8 Hz, 1H), 4.61 (s, 2H), 3.83 (t, J=6.2 Hz, 2H), 3.77-3.56 (m, 6H), 3.16-2.97 (m, 2H), 2.85 (d, J=6.9 Hz, 2H). LCMS (ESI) calcd, for C₂₇H₂₆FN₈O₅⁺ [M+H]⁺: 561.20, found, 561.2.

### Example 24: Preparation of 5-(4-((4-(3-amino-6-(5-fluoro-2-hydroxyphenyl)pyridazin-4-yl)piperazin-1-yl)methyl)piperidin-1-yl)-2-(2,4-dioxotetrahydropyrimidin-1(2H)-yl)isoindoline-1,3-dione (GT-05068)

With reference to the method of Scheme T-3, the target compound (GT-05068) was obtained (white solid, 23 mg, yield 25.84%). ¹H NMR (400 MHz, DMSO) δ 11.11 (s, 1H), 10.85 (s, 1H), 7.74 (t, J=8.0 Hz, 1H), 7.67 (s, 1H), 7.52 (dd, J=9.3, 3.2 Hz, 1H), 7.42 (d, J=2.0 Hz, 1H), 7.36-7.24 (m, 2H), 7.11 (dd, J=9.0, 4.7 Hz, 1H), 4.13 (d, J=12.7 Hz, 2H), 3.80 (d, J=6.8 Hz, 2H), 3.64 (d, J=15.0 Hz, 4H), 3.21 (s, 4H), 3.11-2.98 (m, 4H), 2.83 (t, J=7.2 Hz, 2H), 2.23 (s, 1H), 1.97 (t, J=18.1 Hz, 2H), 1.31 (d, J=11.4 Hz, 2H). LCMS (ESI) calcd, for C₃₂H₃₅FNO₅⁺ [M+H]⁺: 644.27, found, 644.3.

### Example 25: Preparation of 4-(4-((4-(3-amino-6-(5-fluoro-2-hydroxyphenyl)pyridazin-4-yl)piperazin-1-yl)methyl)piperidin-1-yl)-2-(2,4-dioxotetrahydropyrimidin-1(2H)-yl)isoindoline-1,3-dione (GT-05069)

With reference to the method of Scheme T-3, the target compound (GT-05069) was obtained (white solid, 15 mg, yield 16.85%). ¹H NMR (400 MHz, DMSO) δ 11.16 (s, 1H), 10.86 (s, 1H), 7.76 (dd, J=8.5, 7.2 Hz, 1H), 7.68 (s, 1H), 7.52 (dd, J=9.3, 3.1 Hz, 1H), 7.41 (t, J=8.6 Hz, 2H), 7.27 (td, J=8.6, 3.2 Hz, 1H), 7.12 (dd, J=9.0, 4.8 Hz, 1H), 3.80 (td, J=12.1, 5.5 Hz, 4H), 3.67 (d, J=12.9 Hz, 4H), 3.37-3.30 (m, 4H), 3.13 (s, 2H), 2.96 (dt, J=20.1, 12.0 Hz, 2H), 2.84 (dd, J=11.3, 6.5 Hz, 2H), 2.20-1.97 (m, 3H), 1.61-1.39 (m, 2H). LCMS (ESI) calcd, for C₃₂H₃₅FN₉O₅⁺ [M+H]⁺: 644.27, found, 644.3.

### Example 26: Preparation of 5-(4-((4-(3-amino-6-(5-fluoro-2-hydroxyphenyl)pyridazin-4-yl)piperazin-1-yl)methyl)-[1,4′-bipiperidin]-1′-yl)-2-(2,4-dioxotetrahydropyrimidin-1(2H)-yl)isoindoline-1,3-dione (GT-05094)

With reference to the method of Scheme T-3, the target compound (GT-05094) was obtained (yellow solid, 15 mg, yield 14.93%). ¹H NMR (400 MHz, DMSO) δ 11.21 (s, 1H), 10.84 (s, 1H), 10.75 (s, 1H), 7.75 (d, J=8.6 Hz, 1H), 7.65 (s, 1H), 7.57 (d, J=6.9 Hz, 1H), 7.47 (s, 1H), 7.36 (d, J=8.2 Hz, 1H), 7.24 (dd, J=8.3, 5.5 Hz, 1H), 7.07 (dd, J=9.0, 4.8 Hz, 1H), 4.28 (d, J=12.5 Hz, 2H), 3.78 (t, J=6.8 Hz, 2H), 3.73 (s, 1H), 3.63 (s, 10H), 3.10-2.92 (m, 6H), 2.83 (t, J=7.0 Hz, 2H), 2.24-2.07 (m, 5H), 1.75 (d, J=7.8 Hz, 4H). LCMS (ESI) calcd, for C₃₇H₄₄FN₁₀O₅⁺ [M+H]⁺: 727.35, found, 727.4.

### Example 27: Preparation of 2-(6,7-dihydro-5H-pyrrolo[1,2-c]imidazol-1-yl)-2-(6-(4-(4-(((2-(2,4-dioxotetrahydropyrimidin-1(2H)-yl)-1-oxoisoindolin-5-yl)methyl)(methyl)amino)piperidin-1-yl)phenyl)-4-fluoro-1-oxoisoindolin-2-yl)-N-(thiazol-2-yl)acetamide (GT-05099)

With reference to the method of Step 2 in Scheme T-1, the target compound (GT-05099) was obtained (white solid, 11 mg, yield: 15.29%). LCMS (ESI) calcd, for C₄₄H₄₄FN₁₀O₅S⁺ [M+H]⁺: 843.32, found, 843.3.

### Example 28: Preparation of 1-(5-((4-(4-amino-3-(4-phenoxyphenyl)-1H-pyrazolo[3,4-d]pyrimidin-1-yl)piperidin-1-yl)methyl)-1-oxoisoindolin-2-yl)dihydropyrimidine-2,4 (1H,3H)-dione (GT-05075)

With reference to the method of Step 2 in Scheme T-1, the target compound (GT-05075) was obtained (white solid, 50 mg, yield 75.04%). ¹H NMR (400 MHz, DMSO) δ 11.11 (s, 1H), 10.69 (s, 1H), 8.50 (d, J=5.5 Hz, 1H), 7.93 (s, 1H), 7.89 (d, J=7.9 Hz, 1H), 7.82 (d, J=7.9 Hz, 1H), 7.65 (d, J=8.6 Hz, 2H), 7.45 (t, J=7.9 Hz, 2H), 7.24-7.13 (m, 5H), 5.07 (t, J=11.8 Hz, 1H), 4.83 (d, J=16.4 Hz, 1H), 4.65-4.47 (m, 3H), 3.80 (t, J=6.8 Hz, 2H), 3.32 (s, 6H), 2.93-2.76 (m, 2H), 2.67-2.54 (m, 2H), 2.15 (t, J=18.0 Hz, 2H). LCMS (ESI) calcd, for C₃₅H₃₄N₉O₄⁺ [M+H]⁺: 644.27, found, 644.3.

### Example 29: Preparation of 5-((4-(4-amino-3-(4-phenoxyphenyl)-1H-pyrazolo[3,4-d]pyrimidin-1-yl)piperidin-1-yl)methyl)-2-(2,4-dioxotetrahydropyrimidin-1(2H)-yl)isoindoline-1,3-dione (GT-04824)

With reference to the method of Step 2 in Scheme T-1, the target compound (GT-04824) was obtained (white solid, 47 mg, yield 55.24%). ¹H NMR (400 MHz, DMSO) δ 11.19 (s, 1H), 10.92 (s, 1H), 8.47 (s, 1H), 8.34 (s, 1H), 8.16 (dd, J=32.3, 7.7 Hz, 2H), 7.64 (d, J=8.6 Hz, 2H), 7.44 (t, J=7.9 Hz, 2H), 7.16 (ddd, J=12.2, 11.7, 7.6 Hz, 5H), 5.07 (t, J=11.8 Hz, 1H), 4.61 (d, J=14.4 Hz, 2H), 3.83 (t, J=6.7 Hz, 2H), 3.31 (s, 2H), 3.12 (ddd, J=14.7, 7.4, 4.3 Hz, 1H), 2.89 (s, 1H), 2.86 (t, J=6.7 Hz, 2H), 2.73 (s, 1H), 2.63-2.55 (m, 1H), 2.18 (d, J=11.9 Hz, 2H). LCMS (ESI) calcd, for C₃₅H₃₂N₉O₅⁺ [M+H]⁺: 658.25, found, 658.3.

### Example 30: Preparation of 5-(4-((4-(4-amino-3-(4-phenoxyphenyl)-1H-pyrazolo[3,4-d]pyrimidin-1-yl)piperidin-1-yl)methyl)piperidin-1-yl)-2-(2,4-dioxotetrahydropyrimidin-1(2H)-yl)isoindoline-1,3-dione (GT-05076)

With reference to the method of Scheme T-3, the target compound (GT-05076) was obtained (yellow solid, 55 mg, yield 57.38%). ¹H NMR (400 MHz, DMSO) § 10.85 (d, J=8.8 Hz, 1H), 10.14 (s, 1H), 8.51 (s, 1H), 7.70 (dd, J=23.3, 8.6 Hz, 3H), 7.45 (dd, J=15.7, 8.1 Hz, 3H), 7.34-7.29 (m, 1H), 7.18 (td, J=15.0, 7.6 Hz, 5H), 5.10 (s, 1H), 4.13 (d, J=12.6 Hz, 2H), 3.78 (t, J=6.8 Hz, 2H), 3.69 (d, J=11.3 Hz, 4H), 3.27 (dd, J=22.8, 11.3 Hz, 4H), 3.05 (d, J=12.9 Hz, 4H), 2.83 (t, J=7.0 Hz, 2H), 2.68 (dd, J=23.0, 12.5 Hz, 2H), 2.18 (d, J=13.0 Hz, 3H), 1.95 (d, J=11.5 Hz, 2H). LCMS (ESI) calcd, for C₄₀H₄₁N₁₀O₅⁺ [M+H]⁺: 741.33, found, 741.3.

### Example 31: Preparation of 4-(4-((4-(4-amino-3-(4-phenoxyphenyl)-1H-pyrazolo[3,4-d]pyrimidin-1-yl)piperidin-1-yl)methyl)piperidin-1-yl)-2-(2,4-dioxotetrahydropyrimidin-1(2H)-yl)isoindoline-1,3-dione (GT-05077)

With reference to the method of Scheme T-3, the target compound (GT-05077) was obtained (yellow solid, 60 mg, yield 62.60%). ¹H NMR (400 MHz, DMSO) δ 10.85 (s, 1H), 10.35 (s, 1H), 8.60 (s, 1H), 7.78-7.73 (m, 1H), 7.68 (d, J=8.6 Hz, 2H), 7.49-7.37 (m, 4H), 7.19 (td, J=15.0, 7.7 Hz, 5H), 5.12 (t, J=11.9 Hz, 1H), 3.83 (dd, J=11.6, 6.9 Hz, 6H), 3.47-3.38 (m, 2H), 3.36-3.24 (m, 3H), 3.09 (s, 2H), 3.02-2.90 (m, 2H), 2.84 (dd, J=12.6, 6.3 Hz, 2H), 2.72 (dd, J=23.6, 10.8 Hz, 2H), 2.26-1.95 (m, 5H), 1.57-1.41 (m, 2H). LCMS (ESI) calcd, for C₄₀H₄₁N₁₀O₅⁺ [M+H]⁺: 741.33, found, 741.3.

### Example 32: Preparation of 5-(4-((4-(4-amino-3-(4-phenoxyphenyl)-1H-pyrazolo[3,4-d]pyrimidin-1-yl)piperidin-1-yl)methyl)-[1,4′-bipiperidin]-1′-yl)-2-(2,4-dioxotetrahydropyrimidin-1(2H)-yl)isoindoline-1,3-dione (GT-05096)

With reference to the method of Scheme T-3, the target compound (GT-05096) was obtained (yellow solid, 16 mg, yield 15.01%). ¹H NMR (400 MHz, DMSO) δ 10.84 (s, 1H), 10.80 (s, 1H), 10.33 (s, 1H), 8.48 (s, 1H), 7.79-7.63 (m, 3H), 7.50-7.41 (m, 3H), 7.36 (d, J=7.0 Hz, 1H), 7.24-7.09 (m, 5H), 5.08 (s, 1H), 4.28 (d, J=12.3 Hz, 2H), 3.79 (t, J=6.8 Hz, 2H), 3.74-3.62 (m, 4H), 3.24 (d, J=9.5 Hz, 6H), 2.98 (dd, J=27.3, 12.9 Hz, 5H), 2.83 (t, J=6.9 Hz, 2H), 2.67 (d, J=8.0 Hz, 2H), 2.25-2.06 (m, 6H), 1.79-1.63 (m, 3H). LCMS (ESI) calcd, for C₄₅H₅₀N₁₁O₅⁺ [M+H]⁺: 824.40, found, 824.4.

### Example 33: Preparation of 2-(2,4-dioxotetrahydropyrimidin-1(2H)-yl)-5-((4-(4-((1R,2S)-6-hydroxy-2-phenyl-1,2,3,4-tetrahydronaphthalen-1-yl)phenyl)piperazin-1-yl)methyl)isoindoline-1,3-dione (GT-04823)

With reference to the method of Step 2 in Scheme T-1, the target compound (GT-04823) was obtained (white solid, 42 mg, yield 54.73%). ¹H NMR (400 MHz, DMSO) § 11.36 (s, 1H), 10.92 (s, 1H), 8.31 (s, 1H), 8.21-8.05 (m, 2H), 7.13 (ddd, J=10.1, 7.6, 2.4 Hz, 3H), 6.83 (d, J=6.7 Hz, 2H), 6.65-6.56 (m, 4H), 6.48 (dd, J=8.3, 2.5 Hz, 1H), 6.25 (d, J=8.7 Hz, 2H), 4.58 (s, 2H), 4.16 (d, J=4.9 Hz, 1H), 3.81 (t, J=6.8 Hz, 2H), 3.63 (d, J=11.2 Hz, 2H), 3.33 (dd, J=20.6, 13.3 Hz, 4H), 3.12 (dd, J=7.4, 4.2 Hz, 2H), 3.05-2.93 (m, 3H), 2.85 (t, J=6.8 Hz, 2H), 2.14-2.02 (m, 1H), 1.70 (d, J=7.2 Hz, 1H). LCMS (ESI) calcd, for C₃₉H₃₈N₅O₅⁺ [M+H]⁺: 656.29, found, 656.3.

### Example 34: Preparation of 2-(2,4-dioxotetrahydropyrimidin-1(2H)-yl)-5-((4-(4-((3S,4R)-7-hydroxy-3-phenylchroman-4-yl)phenyl)piperazin-1-yl)methyl)isoindoline-1,3-dione (GT-04778)

With reference to the method of Step 2 in Scheme T-1, the target compound (GT-04778) was obtained (white solid, 5 mg, yield 5.34%). ¹H NMR (400 MHz, DMSO) δ 10.92 (d, J=9.6 Hz, 2H), 9.33 (s, 1H), 8.27 (s, 1H), 8.11 (d, J=7.8 Hz, 2H), 7.14 (d, J=2.9 Hz, 3H), 6.78 (s, 2H), 6.70-6.60 (m, 3H), 6.44 (d, J=8.5 Hz, 2H), 6.36-6.24 (m, 2H), 4.59 (s, 2H), 4.33 (t, J=11.3 Hz, 1H), 4.25-4.18 (m, 2H), 3.82 (t, J=6.7 Hz, 2H), 3.68 (s, 2H), 3.53 (d, J=11.9 Hz, 2H), 3.30-3.24 (m, 2H), 3.17 (s, 2H), 2.98 (s, 2H), 2.86 (t, J=6.6 Hz, 2H). LCMS (ESI) calcd, for C₃₈H₃₆N₅O₆⁺ [M+H]⁺: 658.27, found, 658.3.

### Example 35: Preparation of 5-((4-(1-(4-(5,7-dimethoxy-4-oxo-3,4-dihydroquinazolin-2-yl)phenyl)piperidin-4-yl)piperazin-1-yl)methyl)-2-(2,4-dioxotetrahydropyrimidin-1(2H)-yl)isoindoline-1,3-dione (GT-04659)

With reference to the method of Step 2 in Scheme T-1, the target compound (GT-04659) was obtained (white solid, 28 mg, yield 58.21%). ¹H NMR (400 MHz, DMSO) δ 11.03 (s, 1H), 7.19-7.09 (m, 3H), 6.83 (d, J=7.4 Hz, 2H), 6.63 (d, J=8.7 Hz, 2H), 6.49 (d, J=8.2 Hz, 1H), 6.28 (d, J=8.0 Hz, 2H), 5.16 (dd, J=13.1, 5.0 Hz, 1H), 4.45 (d, J=17.7 Hz, 1H), 4.18 (d, J=4.7 Hz, 1H), 3.65 (s, 5H), 3.31 (d, J=12.4 Hz, 4H), 2.95 (dd, J=16.0, 8.4 Hz, 4H), 2.63 (d, J=17.2 Hz, 4H), 2.37 (d, J=9.4 Hz, 1H), 2.11 (s, 2H), 2.02 (dd, J=15.0, 8.3 Hz, 2H), 1.77 (d, J=37.0 Hz, 2H). LCMS (ESI) calcd, for C₃₈H₄₁N₈O₇⁺ [M+H]⁺: 721.31, found, 721.3

### Example 36: Preparation of 8-(4-(4-((2-(2,4-dioxotetrahydropyrimidin-1(2H)-yl)-1,3-dioxoisoindolin-5-yl)methyl)piperazin-1-yl)piperidin-1-yl)-9-ethyl-6,6-dimethyl-11-oxo-6,11-dihydro-5H-benzo[b]carbazole-3-carbonitrile (GT-04661)

With reference to the method of Step 2 in Scheme T-1, the target compound (GT-04661) was obtained (white solid, 30 mg, yield 63.98%). ¹H NMR (400 MHz, DMSO) δ 12.81 (s, 1H), 10.91 (s, 2H), 8.32 (d, J=8.0 Hz, 1H), 8.09-7.99 (m, 5H), 7.60 (d, J=8.4 Hz, 1H), 7.36 (s, 1H), 4.98 (s, 1H), 3.82 (dd, J=12.1, 6.3 Hz, 3H), 3.58 (s, 4H), 3.31 (d, J=10.4 Hz, 2H), 2.85 (dd, J=11.5, 6.7 Hz, 5H), 2.73-2.68 (m, 2H), 2.21 (s, 2H), 1.91 (s, 3H), 1.76 (s, 6H), 1.27 (dd, J=17.3, 9.9 Hz, 6H). LCMS (ESI) calcd, for C₄₃H₄₅N₈O₅⁺ [M+H]⁺: 753.35, found, 753.4.

### Example 37: Preparation of 5-(((1-(4-((5-chloro-4-((2-(dimethylphosphoryl)phenyl)amino)pyrimidin-2-yl)amino)-3-methoxyphenyl)piperidin-4-yl)(methyl)amino)methyl)-2-(2,4-dioxotetrahydropyrimidin-1(2H)-yl)isoindoline-1,3-dione (GT-04660)

With reference to the method of Step 2 in Scheme T-1, the target compound (GT-04660) was obtained (white solid, 50 mg, yield 81.87%). ¹H NMR (400 MHz, DMSO) δ 11.01 (s, 1H), 7.76 (s, 1H), 7.66 (s, 1H), 7.14 (d, J=7.4 Hz, 3H), 6.82 (d, J=7.0 Hz, 2H), 6.74 (s, 2H), 6.67-6.59 (m, 2H), 6.48 (dd, J=8.3, 2.1 Hz, 1H), 6.28 (d, J=7.6 Hz, 2H), 5.14 (dd, J=13.3, 5.0 Hz, 1H), 4.56 (s, 2H), 4.18 (d, J=4.6 Hz, 2H), 3.64 (s, 4H), 3.31 (d, J=12.7 Hz, 5H), 2.96 (s, 4H), 2.69-2.57 (m, 2H), 2.16-2.05 (m, 3H), 1.83 (s, 2H), 1.72 (s, 1H). LCMS (ESI) calcd, for C₃₈H₄₂ClN₉O₆P⁺ [M+H]⁺: 786.27, found, 786.3.

### Example 38: Preparation of N-(5-(4-((2-(2,4-dioxotetrahydropyrimidin-1(2H)-yl)-1-oxoisoindolin-5-yl)methyl)piperazin-1-yl)-1-((1s,4s)-4-(hydroxymethyl)cyclohexyl)-1H-benzo[d]imidazol-2-yl)-3-(trifluoromethyl)benzamide (GT-04949)

With reference to the method of Step 2 in Scheme T-1, the target compound (GT-04949) was obtained (white solid, 19 mg, yield 27.46%). ¹H NMR (400 MHz, DMSO) δ 12.78 (s, 1H), 11.25 (s, 1H), 10.69 (s, 1H), 8.49 (d, J=7.8 Hz, 1H), 8.44 (s, 1H), 7.97-7.86 (m, 3H), 7.83 (d, J=7.8 Hz, 1H), 7.74 (t, J=7.8 Hz, 1H), 7.52 (d, J=8.9 Hz, 1H), 7.20 (s, 1H), 7.01 (d, J=7.1 Hz, 1H), 4.83 (d, J=16.6 Hz, 1H), 4.70 (t, J=12.3 Hz, 1H), 4.66-4.40 (m, 3H), 3.80 (t, J=6.8 Hz, 2H), 3.72 (s, 1H), 3.68 (d, J=7.5 Hz, 3H), 3.45 (s, 2H), 3.29-3.14 (m, 4H), 2.93-2.83 (m, 1H), 2.76 (d, J=22.8 Hz, 1H), 2.43 (d, J=12.0 Hz, 2H), 1.94 (d, J=12.9 Hz, 2H), 1.85 (s, 1H), 1.72-1.57 (m, 4H). LCMS (ESI) calcd, for C₃₉H₄₂F₃N₈O₅⁺ [M+H]⁺: 759.32, found, 759.4.

### Example 39: Preparation of N-(5-(4-(1-((2-(2,4-dioxotetrahydropyrimidin-1(2H)-yl)-1-oxoisoindolin-5-yl)methyl)piperidin-4-yl)piperazin-1-yl)-1-((1s,4s)-4-(hydroxymethyl)cyclohexyl)-1H-benzo[d]imidazol-2-yl)-3-(trifluoromethyl)benzamide (GT-05019)

With reference to the method of Step 2 in Scheme T-1, the target compound (GT-05019) was obtained (white solid, 19 mg, yield 37.29%). ¹H NMR (400 MHz, DMSO-d₆) δ 10.72 (s, 1H), 8.49 (d, J=7.6 Hz, 1H), 8.44 (s, 1H), 7.94-7.86 (m, 3H), 7.75 (dd, J=15.6, 7.8 Hz, 2H), 7.52 (d, J=8.9 Hz, 1H), 7.21 (s, 1H), 7.03 (d, J=9.0 Hz, 1H), 4.82 (d, J=17.0 Hz, 1H), 4.70 (s, 1H), 4.59 (d, J=17.0 Hz, 1H), 4.46 (s, 2H), 3.80 (t, J=6.8 Hz, 3H), 3.72 (d, J=9.7 Hz, 2H), 3.68 (d, J=7.6 Hz, 3H), 3.61 (s, 3H), 3.20 (s, 4H), 2.99 (s, 2H), 2.92-2.84 (m, 1H), 2.79 (s, 1H), 2.46 (s, 1H), 2.35 (d, J=13.7 Hz, 2H), 2.20 (s, 2H), 1.94 (d, J=12.0 Hz, 3H), 1.85 (s, 1H), 1.65 (d, J=14.1 Hz, 4H). LCMS (ESI) calcd, for C₄₄H₅₁F₃N₉O₅⁺ [M+H]⁺: 842.40, found, 842.4.

### Example 40: Preparation of 5-((1R,4R)-2-oxa-5-azabicyclo[2.2.1]heptan-5-yl)-N-(3-(difluoromethyl)-1-(1-((2-(2,4-dioxotetrahydropyrimidin-1(2H)-yl)-1-oxoisoindolin-5-yl)methyl)piperidin-4-yl)-1H-pyrazol-4-yl)pyrazolo[1,5-a]pyrimidine-3-carboxamide (GT-05028)

With reference to the method of Step 2 in Scheme T-1, the target compound (GT-05028) was obtained (white solid, 33 mg, yield 43.44%). ¹H NMR (400 MHz, DMSO-d₆) δ 10.81 (s, 1H), 10.72 (s, 1H), 9.53 (d, J=6.3 Hz, 1H), 8.90-8.74 (m, 1H), 8.43 (d, J=3.2 Hz, 1H), 8.27 (t, J=6.4 Hz, 1H), 7.89 (t, J=7.9 Hz, 2H), 7.79 (d, J=7.6 Hz, 1H), 5.18 (d, J=74.3 Hz, 1H), 4.80 (dd, J=22.9, 18.9 Hz, 2H), 4.68-4.40 (m, 4H), 3.88-3.72 (m, 4H), 3.59 (s, 1H), 3.53 (d, J=9.2 Hz, 2H), 3.45 (d, J=10.5 Hz, 1H), 3.15 (s, 2H), 2.87 (dd, J=16.1, 8.3 Hz, 1H), 2.81-2.70 (m, 1H), 2.33 (d, J=10.2 Hz, 2H), 2.26 (s, 2H), 2.10-1.99 (m, 1H), 1.99-1.90 (m, 1H). LCMS (ESI) calcd, for C₃₄H₃₆F₂N₁₁O₅⁺ [M+H]⁺: 716.29, found, 716.3. LCMS (ESI) calcd. for C₃₄H₃₆F₂N₁₁O₅⁺ [M+H]⁺: 716.29, found, 716.3.

### Example 41: Preparation of N-(5-(4-((1-(2-(2,4-dioxotetrahydropyrimidin-1(2H)-yl)-1,3-dioxoisoindolin-5-yl)piperidin-4-yl)methyl)piperazin-1-yl)-1-((1s,4s)-4-(hydroxymethyl)cyclohexyl)-1H-benzo[d]imidazol-2-yl)-3-(trifluoromethyl)benzamide (GT-05055)

With reference to the method of Scheme T-3, the target compound (GT-05055) was obtained (yellow solid, 20 mg, yield 25.76%). ¹H NMR (400 MHz, DMSO-d₆) δ 10.86 (s, 1H), 10.27 (s, 1H), 8.50 (d, J=7.8 Hz, 1H), 8.45 (s, 1H), 7.91 (d, J=7.7 Hz, 1H), 7.82-7.68 (m, 2H), 7.53 (d, J=8.9 Hz, 1H), 7.43 (s, 1H), 7.34 (d, J=8.7 Hz, 1H), 7.22 (d, J=1.8 Hz, 1H), 7.04 (d, J=8.8 Hz, 1H), 4.71 (s, 1H), 4.15 (d, J=13.0 Hz, 2H), 3.79 (t, J=6.9 Hz, 2H), 3.67 (t, J=10.2 Hz, 7H), 3.30-3.20 (m, 4H), 3.06 (dd, J=25.8, 9.0 Hz, 4H), 2.89-2.79 (m, 2H), 2.47 (s, 1H), 2.22 (s, 1H), 1.95 (d, J=12.4 Hz, 4H), 1.86 (s, 1H), 1.66 (dd, J=26.6, 12.7 Hz, 4H), 1.36-1.21 (m, 2H). LCMS (ESI) calcd, for C₄₄H₅₀F₃N₉O₆⁺ [M+H]⁺: 856.38, found, 856.4.

### Example 42: Preparation of 5-((1R,4R)-2-oxa-5-azabicyclo[2.2.1]heptan-5-yl)-N-(3-(difluoromethyl)-1-(1-((1-(2-(2,4-dioxotetrahydropyrimidin-1(2H)-yl)-1,3-dioxoisoindolin-5-yl)piperidin-4-yl)methyl)piperidin-4-yl)-1H-pyrazol-4-yl)pyrazolo[1,5-a]pyrimidine-3-carboxamide (GT-05056)

With reference to the method of Scheme T-, the target compound (GT-05056) was obtained (white solid, 37 mg, yield 43.13%). ¹H NMR (400 MHz, DMSO-d₆) δ 10.86 (d, J=3.0 Hz, 1H), 9.62 (s, 1H), 9.55 (d, J=6.8 Hz, 1H), 8.80 (dd, J=7.8, 1.7 Hz, 1H), 8.46 (d, J=3.4 Hz, 1H), 8.27 (d, J=5.6 Hz, 1H), 7.72 (d, J=8.2 Hz, 1H), 7.44-7.40 (m, 1H), 7.31 (dd, J=9.5, 7.3 Hz, 1H), 4.77 (d, J=21.5 Hz, 1H), 4.58 (s, 1H), 4.15 (d, J=12.6 Hz, 2H), 3.97 (d, J=13.4 Hz, 1H), 3.82-3.76 (m, 4H), 3.66 (t, J=12.0 Hz, 2H), 3.60 (s, 1H), 3.21 (d, J=10.5 Hz, 1H), 3.13 (dd, J=19.7, 9.5 Hz, 2H), 3.04 (d, J=10.6 Hz, 3H), 2.86-2.79 (m, 3H), 2.41-2.31 (m, 2H), 2.26 (d, J=13.0 Hz, 2H), 2.05-1.98 (m, 1H), 1.93 (d, J=12.3 Hz, 4H), 1.62-1.48 (m, 1H), 1.28 (d, J=12.1 Hz, 2H). LCMS (ESI) calcd, for C₃₉H₄₃F₂N₁₂O₆⁺ [M+H]⁺: 813.34, found, 813.4.

### Example 43: Preparation of 2-(6,7-dihydro-5H-pyrrolo[1,2-c]imidazol-1-yl)-2-(6-(4-((1S,4S)-5-((2-(2,4-dioxotetrahydropyrimidin-1(2H)-yl)-1-oxoisoindolin-5-yl)methyl)-2,5-diazabicyclo[2.2.1]heptan-2-yl)phenyl)-4-fluoro-1-oxoisoindolin-2-yl)-N-(thiazol-2-yl)acetamide (GT-05114)

With reference to the method of Step 2 in Scheme T-1, the target compound (GT-05114) was obtained (white solid, 27 mg, yield 50.44%). ¹H NMR (400 MHz, DMSO-d₆) δ 10.70 (s, 1H), 9.03 (s, 1H), 7.99 (s, 1H), 7.91-7.82 (m, 2H), 7.81-7.72 (m, 2H), 7.53 (d, J=3.6 Hz, 1H), 7.33 (d, J=3.5 Hz, 1H), 6.74 (d, J=8.8 Hz, 1H), 6.37 (s, 1H), 4.90-4.59 (m, 4H), 4.58-4.44 (m, 3H), 4.24 (t, J=6.7 Hz, 2H), 3.77 (dd, J=13.5, 7.0 Hz, 3H), 3.65 (d, J=9.8 Hz, 1H), 3.44 (s, 3H), 3.22 (d, J=11.9 Hz, 1H), 2.92-2.80 (m, 2H), 2.80-2.68 (m, 1H), 2.67-2.52 (m, 4H), 2.20 (d, J=11.4 Hz, 1H). LCMS (ESI) calcd, for C₄₃H₄₀FN₁₀O₅S⁺ [M+H]⁺: 827.29, found, 827.3.

### Example 44: Preparation of 7-cyclopentyl-2-((5-(4-((4-(2-(2,4-dioxotetrahydropyrimidin-1(2H)-yl)-1,3-dioxoisoindolin-5-yl)piperazin-1-yl)methyl)piperidin-1-yl)pyridin-2-yl)amino)-N,N-dimethyl-7H-pyrrolo[2,3-d]pyrimidine-6-carboxamide (GT-05121)

With reference to the method of Scheme T-5, the target compound (GT-05121) was obtained (yellow solid, 56 mg, yield 62.82%). ¹H NMR (400 MHz, DMSO) δ 11.46 (s, 1H), 10.88 (d, J=19.3 Hz, 2H), 9.00 (s, 1H), 8.14 (d, J=9.5 Hz, 1H), 7.93 (s, 1H), 7.83 (d, J=8.5 Hz, 1H), 7.62-7.51 (m, 2H), 7.42 (d, J=6.9 Hz, 1H), 6.83 (s, 1H), 4.96-4.75 (m, 1H), 4.23 (d, J=12.4 Hz, 2H), 3.80 (t, J=6.9 Hz, 3H), 3.71 (d, J=12.5 Hz, 2H), 3.62 (s, 4H), 3.11 (s, 2H), 3.07 (s, 7H), 2.84 (dd, J=12.8, 5.9 Hz, 4H), 2.33 (d, J=1.9 Hz, 2H), 2.07-1.97 (m, 6H), 1.70-1.62 (m, 2H), 1.40 (d, J=9.8 Hz, 2H). LCMS (ESI) calcd, for C₄₁H₄₉N₁₂O₅⁺ [M+H]⁺: 789.39, found, 789.4.

### Example 45: Preparation of 5-((1R,4R)-2-oxa-5-azabicyclo[2.2.1]heptan-5-yl)-N-(3-(difluoromethyl)-1-((1r,4R)-4-((4-(1-(2-(2,4-dioxotetrahydropyrimidin-1(2H)-yl)-1,3-dioxoisoindolin-5-yl)piperidin-4-yl)piperazin-1-yl)methyl)cyclohexyl)-1H-pyrazol-4-yl)pyrazolo[1,5-a]pyrimidine-3-carboxamide (GT-05122)

With reference to the method of Scheme T-5, the target compound (GT-05122) was obtained (yellow solid, 10.5 mg, yield 9.47%). ¹H NMR (400 MHz, DMSO) δ 10.85 (s, 1H), 9.52 (s, 1H), 8.78 (d, J=7.4 Hz, 1H), 8.40 (s, 1H), 8.26 (d, J=4.6 Hz, 1H), 7.76 (d, J=8.8 Hz, 1H), 7.47 (s, 1H), 7.36 (d, J=8.1 Hz, 1H), 6.93 (d, J=40.9 Hz, 1H), 6.46 (d, J=6.4 Hz, 1H), 4.77 (d, J=19.6 Hz, 1H), 4.26 (s, 3H), 3.78 (dd, J=16.0, 9.4 Hz, 6H), 3.60 (s, 4H), 3.01 (s, 4H), 2.84 (d, J=6.8 Hz, 4H), 2.74 (s, 1H), 2.68 (s, 1H), 2.15 (s, 2H), 2.01 (d, J=16.0 Hz, 6H), 1.78 (d, J=12.8 Hz, 6H), 1.18 (s, 3H). LCMS (ESI) calcd, for C₄₄H₅₂F₂N₁₃O₆⁺ [M+H]⁺: 896.41, found, 896.4.

### Example 46: Preparation of 5-((1R,4R)-2-oxa-5-azabicyclo[2.2.1]heptan-5-yl)-N-(3-(difluoromethyl)-1-((1r,4R)-4-((4-(1-(2-(2,4-dioxotetrahydropyrimidin-1(2H)-yl)-1,3-dioxoisoindolin-5-yl)-3,3-difluoropiperidin-4-yl)piperazin-1-yl)methyl)cyclohexyl)-1H-pyrazol-4-yl)pyrazolo[1,5-alpyrimidine-3-carboxamide (GT-05123)

With reference to the method of Scheme T-5, the target compound was (GT-05123) obtained (yellow solid, 30 mg, yield 26.09%). ¹H NMR (400 MHz, DMSO) δ 10.84 (s, 1H), 9.55-9.36 (m, 2H), 8.78 (d, J=7.7 Hz, 1H), 8.40 (d, J=4.2 Hz, 1H), 8.25 (d, J=5.4 Hz, 1H), 7.75 (d, J=8.5 Hz, 1H), 7.51 (s, 1H), 7.41 (d, J=8.6 Hz, 1H), 7.18 (d, J=53.7 Hz, 1H), 7.01-6.80 (m, 1H), 4.77 (d, J=20.8 Hz, 1H), 4.48 (s, 1H), 4.25 (d, J=15.0 Hz, 2H), 3.83-3.76 (m, 3H), 3.74 (d, J=4.8 Hz, 1H), 3.68-3.56 (m, 2H), 3.45 (d, J=9.8 Hz, 4H), 3.17 (s, 2H), 3.02 (d, J=23.8 Hz, 7H), 2.83 (t, J=6.9 Hz, 2H), 2.06 (t, J=10.6 Hz, 2H), 1.93 (dd, J=27.7, 14.8 Hz, 6H), 1.82-1.66 (m, 2H), 1.26-1.10 (m, 2H). LCMS (ESI) calcd, for C₄₄H₅₀F₄N₁₃O₆⁺ [M+H]⁺: 932.39, found, 932.4.

### Example 47: Preparation of N-(2-(1-((2-(2,4-dioxotetrahydropyrimidin-1(2H)-yl)-1-oxoisoindolin-5-yl)methyl)piperidin-4-yl)-6-methoxy-2H-indazol-7-yl)-3-(trifluoromethyl)benzamide (GT-04884)

With reference to the method of Step 2 in Scheme T-1, the target compound (GT-04884) was obtained (white solid, 25 mg, yield 34.40%). ¹H NMR (400 MHz, DMSO) δ 10.68 (s, 2H), 10.09 (s, 1H), 8.40 (s, 2H), 7.97 (d, J=8.2 Hz, 1H), 7.90-7.84 (m, 2H), 7.78 (t, J=10.5 Hz, 3H), 7.70 (d, J=9.4 Hz, 1H), 7.11 (d, J=9.1 Hz, 1H), 4.77 (d, J=11.7 Hz, 2H), 4.48 (s, 2H), 3.82-3.75 (m, 5H), 3.52 (d, J=11.1 Hz, 2H), 3.33-3.29 (m, 1H), 3.19 (d, J=8.8 Hz, 2H), 2.87-2.73 (m, 2H), 2.44 (d, J=13.0 Hz, 2H), 2.30 (d, J=12.6 Hz, 2H). LCMS (ESI) calcd, for C₃₄H₃₃F₃N₇O₅⁺ [M+H]⁺: 676.25, found, 676.3.

### Example 48: Preparation of N-(2-(1-((1-(2-(2,4-dioxotetrahydropyrimidin-1(2H)-yl)-1,3-dioxoisoindolin-5-yl)piperidin-4-yl)methyl)piperidin-4-yl)-6-methoxy-2H-indazol-7-yl)-3-(trifluoromethyl)benzamide (GT-04885)

With reference to the method of Scheme T-3, the target compound (GT-04885) was obtained (white solid, 25 mg, yield 33.84%). ¹H NMR (400 MHz, DMSO) δ 10.83 (s, 1H), 10.10 (s, 1H), 9.62 (s, 1H), 8.48-8.33 (m, 2H), 7.98 (d, J=7.2 Hz, 1H), 7.79 (s, 1H), 7.72 (dd, J=8.8, 2.4 Hz, 2H), 7.41 (d, J=2.1 Hz, 1H), 7.31 (dd, J=8.7, 2.1 Hz, 1H), 7.12 (d, J=9.2 Hz, 1H), 4.77 (s, 1H), 4.12 (d, J=12.8 Hz, 2H), 3.88-3.59 (m, 7H), 3.19-3.12 (m, 2H), 3.07-3.00 (m, 3H), 2.83 (t, J=7.0 Hz, 2H), 2.42-2.13 (m, 5H), 1.89 (d, J=12.3 Hz, 2H), 1.36-1.19 (m, 3H). LCMS (ESI) calcd, for C₃₉H₄₀F₃N₈O₆⁺ [M+H]⁺: 773.30, found, 773.3.

### Example 49: Preparation of N-(2-(1-((1-(2-(2,4-dioxotetrahydropyrimidin-1(2H)-yl)-1,3-dioxoisoindolin-5-yl)piperidin-4-yl)methyl)piperidin-4-yl)-6-methoxy-2H-indazol-7-yl)-3-(trifluoromethyl)benzamide (GT-04885)

With reference to the method of Scheme T-3, the target compound (GT-04885) was obtained (white solid, 25 mg, yield 33.84%). ¹H NMR (400 MHz, DMSO) δ 10.83 (s, 1H), 10.10 (s, 1H), 9.62 (s, 1H), 8.48-8.33 (m, 2H), 7.98 (d, J=7.2 Hz, 1H), 7.79 (s, 1H), 7.72 (dd, J=8.8, 2.4 Hz, 2H), 7.41 (d, J=2.1 Hz, 1H), 7.31 (dd, J=8.7, 2.1 Hz, 1H), 7.12 (d, J=9.2 Hz, 1H), 4.77 (s, 1H), 4.12 (d, J=12.8 Hz, 2H), 3.88-3.59 (m, 7H), 3.19-3.12 (m, 2H), 3.07-3.00 (m, 3H), 2.83 (t, J=7.0 Hz, 2H), 2.42-2.13 (m, 5H), 1.89 (d, J=12.3 Hz, 2H), 1.36-1.19 (m, 3H). LCMS (ESI) calcd, for C₃₉H₄₀F₃N₈O₆⁺ [M+H]⁺: 773.30, found, 773.3.

### Example 50: Preparation of N-(5-(4-((2-(2,4-dioxotetrahydropyrimidin-1(2H)-yl)-1,3-dioxoisoindolin-5-yl)methyl)piperazin-1-yl)-1-((1s,4s)-4-(hydroxymethyl)cyclohexyl)-1H-benzo[d]imidazol-2-yl)-3-(trifluoromethyl)benzamide (GT-05081)

With reference to the method of Scheme T-1, the target compound (GT-05081) was obtained (white solid, 25 mg, yield 40.56%). ¹H NMR (400 MHz, DMSO) δ 12.79 (s, 1H), 11.68 (s, 1H), 11.02-10.85 (m, 1H), 8.49 (d, J=7.7 Hz, 1H), 8.45-7.99 (m, 4H), 7.90 (d, J=7.9 Hz, 1H), 7.74 (t, J=7.7 Hz, 1H), 7.52 (d, J=8.9 Hz, 1H), 7.20 (s, 1H), 6.99 (t, J=14.7 Hz, 1H), 4.67 (d, J=26.1 Hz, 2H), 3.84 (t, J=6.9 Hz, 2H), 3.68 (d, J=7.4 Hz, 6H), 3.35-3.08 (m, 6H), 2.86 (dd, J=13.8, 6.9 Hz, 2H), 2.46 (s, 1H), 1.91 (t, J=21.0 Hz, 3H), 1.66 (dd, J=24.9, 11.4 Hz, 4H). LCMS (ESI) calcd, for C₃₉H₄₀F₃N₈O₆⁺ [M+H]⁺: 773.30, found, 773.3.

### Example 51: Preparation of N-(3-(difluoromethyl)-1-(1-((2-(2,4-dioxotetrahydropyrimidin-1(2H)-yl)-1-oxoisoindolin-5-yl)methyl)piperidin-4-yl)-1H-pyrazol-4-yl)-2-(2-((2,2,2-trifluoroethyl)amino)pyridin-4-yl) oxazole-4-carboxamide (GT-05184)

With reference to the method of Scheme T-1, the target compound (GT-05184) was obtained (white solid, 32 mg, yield 50.43%). ¹H NMR (400 MHz, DMSO) δ 11.14 (s, 1H), 10.70 (s, 1H), 9.89 (d, J=5.7 Hz, 1H), 9.03 (d, J=9.4 Hz, 1H), 8.28-8.15 (m, 2H), 8.10 (s, 1H), 7.88 (tt, J=23.5, 11.8 Hz, 3H), 7.26 (ddd, J=66.0, 53.4, 29.6 Hz, 3H), 4.67 (dd, J=59.9, 32.2 Hz, 3H), 4.49 (s, 2H), 4.41-4.24 (m, 2H), 3.80 (t, J=6.8 Hz, 2H), 3.51 (s, 2H), 3.16 (s, 2H), 2.95-2.70 (m, 2H), 2.48-2.22 (m, 4H). LCMS (ESI) calcd, for C₃₃H₃₂F₅N₁₀O₅⁺ [M+H]⁺: 743.25, found, 743.3.

### Example 52: Preparation of N-(3-(difluoromethyl)-1-(4-(4-((2-(2,4-dioxotetrahydropyrimidin-1(2H)-yl)-1-oxoisoindolin-5-yl)methyl)piperazin-1-yl)phenyl)-1H-pyrazol-4-yl)-2-(2-((2,2,2-trifluoroethyl)amino)pyridin-4-yl) oxazole-4-carboxamide (GT-05318)

With reference to the method of Scheme T-1, the target compound (GT-05318) was obtained (white solid, 30 mg, yield 11.25%). ¹H NMR (400 MHz, DMSO) δ 11.37 (s, 1H), 10.70 (s, 1H), 10.02 (s, 1H), 9.03 (s, 1H), 8.70 (s, 1H), 8.25 (d, J=5.4 Hz, 1H), 7.89 (dd, J=22.3, 14.5 Hz, 4H), 7.74 (d, J=9.1 Hz, 2H), 7.43-7.12 (m, 5H), 4.55 (s, 4H), 4.30 (dd, J=9.5, 6.1 Hz, 2H), 3.90 (d, J=9.9 Hz, 2H), 3.79 (d, J=6.8 Hz, 2H), 3.44 (s, 2H), 3.23 (s, 4H), 2.96-2.72 (m, 2H). LCMS (ESI) calcd, for C₃₈H₃₅F₅N₁₁O₅⁺ [M+H]⁺: 820.27, found, 820.3.

### Example 53: Preparation of 6-(5-cyano-1H-pyrrolo[2,3-b]pyridin-1-yl)-N-(1-((2-(2,4-dioxotetrahydropyrimidin-1(2H)-yl)-1-oxoisoindolin-5-yl)methyl)piperidin-4-yl)-4-((isopropyl)amino) nicotinamide (GT-05319)

With reference to the method of Scheme T-1, the target compound (GT-05319) was obtained (white solid, 13 mg, yield 19.85%). ¹H NMR (400 MHz, DMSO) δ 10.82 (s, 1H), 10.70 (s, 1H), 8.82 (dd, J=5.8, 2.0 Hz, 1H), 8.79-8.61 (m, 3H), 8.52 (dd, J=7.7, 3.9 Hz, 1H), 8.07 (d, J=11.9 Hz, 1H), 7.90 (t, J=8.4 Hz, 2H), 7.80 (d, J=8.0 Hz, 1H), 6.92 (dd, J=10.6, 3.9 Hz, 1H), 4.71 (dd, J=92.1, 16.5 Hz, 2H), 4.46 (dd, J=24.5, 4.6 Hz, 2H), 4.08 (d, J=67.1 Hz, 1H), 3.80 (t, J=6.6 Hz, 3H), 3.27 (s, 2H), 3.13 (dd, J=22.4, 10.7 Hz, 2H), 2.93-2.71 (m, 2H), 2.01 (dd, J=32.7, 18.4 Hz, 4H), 1.29 (d, J=6.3 Hz, 6H). LCMS (ESI) calcd, for C₃₅H₃₇N₁₀O₄⁺ [M+H]⁺: 661.30, found, 661.3.

### Example 54: Preparation of N-(3-(difluoromethyl)-1-(1-((2-(2,4-dioxotetrahydropyrimidin-1(2H)-yl)-1,3-dioxoisoindolin-5-yl)methyl)piperidin-4-yl)-1H-pyrazol-4-yl)-2-(2-((2,2,2-trifluoroethyl)amino)pyridin-4-yl) oxazole-4-carboxamide (GT-05188)

With reference to the method of Scheme T-1, the target compound (GT-05188) was obtained (white solid, 127 mg, yield 34.44%). ¹H NMR (400 MHz, DMSO) δ 11.12 (s, 1H), 10.93 (s, 1H), 9.86 (s, 1H), 9.01 (d, J=8.6 Hz, 1H), 8.43-8.30 (m, 1H), 8.23 (dd, J=11.1, 6.2 Hz, 2H), 8.13 (d, J=7.7 Hz, 1H), 7.91 (s, 1H), 7.35-7.07 (m, 3H), 4.63 (d, J=34.1 Hz, 3H), 4.29 (dd, J=9.9, 5.7 Hz, 2H), 3.83 (t, J=6.7 Hz, 2H), 3.68 (d, J=6.5 Hz, 1H), 3.43-3.36 (m, 1H), 3.18 (s, 2H), 2.86 (t, J=6.8 Hz, 2H), 2.42-2.24 (m, 4H). LCMS (ESI) calcd. for C₃₃H₃₀F₅N₁₀O₆⁺ [M+H]⁺: 757.23, found, 757.3.

### Example 55: Preparation of 2-(6,7-dihydro-5H-pyrrolo[1,2-c]imidazol-1-yl)-2-(6-(4-(4-(((2-(2,4-dioxotetrahydropyrimidin-1(2H)-yl)-1,3-dioxoisoindolin-5-yl)methyl)(methyl)amino)piperidin-1-yl)phenyl)-4-fluoro-1-oxoisoindolin-2-yl)-N-(thiazol-2-yl)acetamide (GT-05196)

With reference to the method of Scheme T-1, the target compound (GT-05196) was obtained (white solid, 13 mg, yield 17.77%). ¹H NMR (400 MHz, DMSO) δ 11.20 (d, J=29.4 Hz, 1H), 10.93 (s, 1H), 9.08 (s, 1H), 8.38 (s, 1H), 8.26 (d, J=8.1 Hz, 1H), 8.10 (d, J=7.8 Hz, 1H), 7.82 (d, J=7.5 Hz, 2H), 7.73 (t, J=9.9 Hz, 2H), 7.53 (d, J=3.2 Hz, 1H), 7.33 (d, J=3.3 Hz, 1H), 7.14 (d, J=8.1 Hz, 2H), 6.39 (s, 1H), 4.77 (t, J=20.7 Hz, 2H), 4.51 (d, J=17.3 Hz, 2H), 4.26 (t, J=7.0 Hz, 2H), 4.00 (s, 2H), 3.83 (t, J=6.6 Hz, 2H), 3.62 (d, J=6.8 Hz, 3H), 3.12 (dd, J=7.5, 4.0 Hz, 1H), 2.86 (t, J=6.6 Hz, 4H), 2.66-2.58 (m, 4H), 2.31 (d, J=13.4 Hz, 2H), 1.94 (s, 2H). LCMS (ESI) calcd, for C₄₄H₄₂FN₁₀O₆S⁺ [M+H]⁺: 857.30, found, 857.3.

### Example 56: Preparation of N-(2-(1-((1-(2-(2,4-dioxotetrahydropyrimidin-1(2H)-yl)-1,3-dioxoisoindolin-5-yl)piperidin-4-yl)methyl)piperidin-4-yl)-6-(2-hydroxypropan-2-yl)-2H-indazol-5-yl)-6-(trifluoromethyl) picolinamide (GT-05220)

With reference to the method of Scheme T-3, the target compound (GT-05220) was obtained (yellow solid, 20 mg, yield 22.32%). ¹H NMR (400 MHz, DMSO) δ 12.39 (s, 1H), 10.85 (s, 1H), 10.16 (s, 1H), 8.76 (s, 1H), 8.55-8.36 (m, 3H), 8.17 (d, J=8.6 Hz, 1H), 7.73 (dd, J=8.6, 4.2 Hz, 1H), 7.61 (d, J=3.6 Hz, 1H), 7.43 (d, J=2.1 Hz, 1H), 7.33 (dd, J=8.8, 2.3 Hz, 1H), 4.87 (d, J=47.8 Hz, 1H), 4.14 (d, J=13.2 Hz, 2H), 3.82-3.69 (m, 5H), 3.29-2.96 (m, 7H), 2.83 (t, J=7.3 Hz, 2H), 2.61 (d, J=12.1 Hz, 2H), 2.34 (dd, J=8.8, 6.5 Hz, 2H), 2.23 (s, 1H), 1.96 (d, J=12.0 Hz, 2H), 1.63 (s, 6H). LCMS (ESI) calcd, for C₄₀H₄₃F₃N₉O₆⁺ [M+H]⁺: 802.33, found, 802.4.

### Example 57: Preparation of N-(2-(1-((1-(2-(2,4-dioxotetrahydropyrimidin-1(2H)-yl)-1,3-dioxoisoindolin-4-yl)piperidin-4-yl)methyl)piperidin-4-yl)-6-(2-hydroxypropan-2-yl)-2H-indazol-5-yl)-6-(trifluoromethyl) picolinamide (GT-05221)

With reference to the method of Scheme T-3, the target compound (GT-05221) was obtained (yellow solid, 15 mg, yield 16.74%). ¹H NMR (400 MHz, DMSO) δ 12.39 (s, 1H), 10.86 (d, J=2.9 Hz, 1H), 10.30 (s, 1H), 8.76 (d, J=2.9 Hz, 1H), 8.55-8.34 (m, 3H), 8.17 (d, J=7.8 Hz, 1H), 7.81-7.71 (m, 1H), 7.61 (d, J=4.4 Hz, 1H), 7.41 (dd, J=13.7, 6.6 Hz, 2H), 4.85 (t, J=29.7 Hz, 1H), 3.81-3.72 (m, 6H), 3.18 (dd, J=29.4, 17.6 Hz, 4H), 3.01-2.82 (m, 4H), 2.64 (dd, J=23.6, 7.2 Hz, 2H), 2.35 (d, J=14.3 Hz, 2H), 2.16-1.96 (m, 4H), 1.63 (s, 6H), 1.54-1.41 (m, 2H). LCMS (ESI) calcd, for C₄₀H₄₃F₃N₉O₆⁺ [M+H]⁺: 802.33, found, 802.3.

### Example 58: Preparation of N-(2-(1-((1-(2-(2,4-dioxotetrahydropyrimidin-1(2H)-yl)-1,3-dioxoisoindolin-5-yl)piperidin-4-yl)methyl)piperidin-4-yl)-5-(2-hydroxypropan-2-yl)benzo[d]oxazol-6-yl)-6-(trifluoromethyl) picolinamide (GT-05222)

With reference to the method of Scheme T-3, the target compound (GT-05222) was obtained (yellow solid, 22 mg, yield 24.58%). ¹H NMR (400 MHz, DMSO) δ 12.64 (s, 1H), 10.85 (s, 1H), 8.76 (d, J=3.6 Hz, 1H), 8.42 (dt, J=20.9, 7.9 Hz, 2H), 8.20 (d, J=7.8 Hz, 1H), 7.80-7.66 (m, 2H), 7.42 (s, 1H), 7.33 (d, J=6.9 Hz, 1H), 4.13 (d, J=12.6 Hz, 2H), 3.79 (t, J=6.7 Hz, 2H), 3.63 (s, 4H), 3.34 (d, J=11.1 Hz, 4H), 3.20-2.98 (m, 6H), 2.83 (t, J=7.0 Hz, 2H), 2.33 (s, 2H), 1.93 (t, J=14.3 Hz, 2H), 1.79-1.48 (m, 6H). LCMS (ESI) calcd, for C₄₀H₄₂F₃N₈O₇⁺ [M+H]⁺: 803.31, found, 803.4.

### Example 59: Preparation of N-(2-(1-((1-(2-(2,4-dioxotetrahydropyrimidin-1(2H)-yl)-1,3-dioxoisoindolin-4-yl)piperidin-4-yl)methyl)piperidin-4-yl)-5-(2-hydroxypropan-2-yl)benzo[d]oxazol-6-yl)-6-(trifluoromethyl) picolinamide (GT-05223)

With reference to the method of Scheme T-3, the target compound (GT-05223) was obtained (yellow solid, 32 mg, yield 35.75%). ¹H NMR (400 MHz, DMSO) δ 12.64 (s, 1H), 10.87 (s, 1H), 9.93 (s, 1H), 8.53-8.29 (m, 2H), 8.22-8.14 (m, 1H), 7.82-7.70 (m, 2H), 7.41 (t, J=8.2 Hz, 2H), 3.73 (dd, J=51.7, 15.0 Hz, 6H), 3.12 (d, J=23.6 Hz, 3H), 3.02-2.70 (m, 6H), 2.32 (dd, J=38.0, 21.3 Hz, 3H), 2.03 (dt, J=33.9, 25.2 Hz, 5H), 1.53 (dd, J=34.2, 28.2 Hz, 7H). LCMS (ESI) calcd, for C₄₀H₄₂F₃N₈O₇⁺ [M+H]⁺: 803.31, found, 803.3.

### Example 60: Preparation of N-(3-(difluoromethyl)-1-(1-((1-(2-(2,4-dioxotetrahydropyrimidin-1(2H)-yl)-1,3-dioxoisoindolin-4-yl)piperidin-4-yl)methyl)piperidin-4-yl)-1H-pyrazol-4-yl)-5-morpholinopyrazolo[1,5-a]pyrimidine-3-carboxamide (GT-05224)

With reference to the method of Scheme T-3, the target compound (GT-05224) was obtained (yellow solid, 32 mg, yield 35.68%). ¹H NMR (400 MHz, DMSO) δ 10.86 (s, 1H), 10.35 (s, 1H), 9.44 (s, 1H), 8.83 (d, J=7.9 Hz, 1H), 8.30 (s, 1H), 7.75 (t, J=7.8 Hz, 1H), 7.41 (t, J=8.6 Hz, 2H), 7.15 (t, J=53.5 Hz, 1H), 6.92 (d, J=8.0 Hz, 1H), 4.59 (s, 1H), 3.96-3.75 (m, 13H), 3.62-3.23 (m, 4H), 3.13 (dd, J=26.2, 14.0 Hz, 4H), 3.02-2.89 (m, 2H), 2.84 (dd, J=11.1, 6.6 Hz, 2H), 2.26 (d, J=12.1 Hz, 2H), 2.03 (s, 3H), 1.45 (s, 2H). LCMS (ESI) calcd, for C₃₈H₄₃F₂N₁₂O₆⁺ [M+H]⁺: 801.34, found, 801.4.

### Example 61: Preparation of 5-((1R,4R)-2-oxa-5-azabicyclo[2.2.1]heptan-5-yl)-N-(3-(difluoromethyl)-1-(1-((1-(2-(2,4-dioxotetrahydropyrimidin-1(2H)-yl)-1,3-dioxoisoindolin-4-yl)piperidin-4-yl)methyl)piperidin-4-yl)-1H-pyrazol-4-yl)pyrazolo[1,5-a]pyrimidine-3-carboxamide (GT-05285)

With reference to the method of Scheme T-3, the target compound (GT-05285) was obtained (yellow solid, 15 mg, yield 16.92%). ¹H NMR (400 MHz, DMSO) § 10.86 (s, 1H), 10.37 (s, 1H), 9.54 (d, J=6.1 Hz, 1H), 8.79 (d, J=7.7 Hz, 1H), 8.46 (d, J=3.0 Hz, 1H), 8.27 (d, J=5.4 Hz, 1H), 7.75 (t, J=7.8 Hz, 1H), 7.41 (t, J=8.5 Hz, 2H), 7.17 (d, J=3.5 Hz, 1H), 6.67 (dd, J=166.0, 7.8 Hz, 1H), 5.19 (d, J=79.0 Hz, 1H), 4.78 (d, J=18.6 Hz, 1H), 4.59 (s, 1H), 3.97-3.72 (m, 7H), 3.63-3.37 (m, 6H), 3.14 (dd, J=26.1, 14.4 Hz, 4H), 3.02-2.89 (m, 2H), 2.84 (dd, J=11.1, 6.4 Hz, 2H), 2.52 (s, 1H), 2.39 (s, 1H), 2.08 (ddd, J=47.5, 30.8, 12.6 Hz, 6H), 1.58-1.36 (m, 2H). LCMS (ESI) calcd, for C₃₉H₄₃F₂N₁₂O₆⁺ [M+H]⁺: 813.34, found, 813.4.

### Example 62: Preparation of N-(2-(1-((1′-(2-(2,4-dioxotetrahydropyrimidin-1(2H)-yl)-1,3-dioxoisoindolin-5-yl)-[1,4′-bipiperidin]-4-yl)methyl)piperidin-4-yl)-6-(2-hydroxypropan-2-yl)-2H-indazol-5-yl)-6-(trifluoromethyl) picolinamide (GT-05290)

With reference to the method of Scheme T-3, the target compound (GT-05290) was obtained (yellow solid, 11 mg, yield 13.90%). ¹H NMR (400 MHz, DMSO) δ 11.19 (s, 1H), 9.94 (s, 1H), 9.03 (s, 1H), 8.78 (t, J=16.1 Hz, 3H), 8.15 (d, J=6.8 Hz, 1H), 8.07-7.89 (m, 4H), 7.72-7.66 (m, 2H), 5.99 (s, 1H), 5.06 (d, J=110.0 Hz, 1H), 4.46 (d, J=7.8 Hz, 2H), 4.28 (s, 1H), 3.86 (s, 6H), 3.25 (d, J=13.0 Hz, 4H), 3.15-3.09 (m, 6H), 2.69 (d, J=8.5 Hz, 6H), 2.10 (dd, J=43.9, 14.5 Hz, 3H), 1.92 (d, J=5.6 Hz, 2H), 1.77 (s, 1H). LCMS (ESI) calcd. for C₄₅H₅₂F₃N₁₀O₆⁺ [M+H]⁺: 885.40, found, 885.5.

### Example 63: Preparation of N-(3-(difluoromethyl)-1-(1-((1-(2-(2,4-dioxotetrahydropyrimidin-1(2H)-yl)-1,3-dioxoisoindolin-5-yl)piperidin-4-yl)methyl)piperidin-4-yl)-1H-pyrazol-4-yl)-5-morpholinopyrazolo[1,5-a]pyrimidine-3-carboxamide (GT-05291)

With reference to the method of Scheme T-3, the target compound (GT-05291) was obtained (yellow solid, 26 mg, yield 28.99%). ¹H NMR (400 MHz, DMSO) δ 10.85 (s, 1H), 9.74 (s, 1H), 9.44 (s, 1H), 8.83 (d, J=7.9 Hz, 1H), 8.44 (d, J=10.9 Hz, 1H), 8.30 (s, 1H), 7.73 (d, J=8.7 Hz, 1H), 7.42 (s, 1H), 7.32 (d, J=9.0 Hz, 1H), 7.15 (t, J=53.4 Hz, 1H), 6.92 (d, J=7.9 Hz, 1H), 4.58 (s, 1H), 4.13 (d, J=13.4 Hz, 1H), 3.91-3.64 (m, 15H), 3.08 (dd, J=24.8, 12.1 Hz, 6H), 2.83 (t, J=6.9 Hz, 2H), 2.75 (t, J=6.3 Hz, 1H), 2.44-2.23 (m, 4H), 1.92 (d, J=7.3 Hz, 2H). LCMS (ESI) calcd, for C₃₈H₄₃F₂N₁₂O₆⁺ [M+H]⁺: 801.34, found, 801.4.

### Example 64: Preparation of N-(5-(4-((1-(2-(2,4-dioxotetrahydropyrimidin-1(2H)-yl)-1,3-dioxoisoindolin-4-yl)piperidin-4-yl)methyl)piperazin-1-yl)-1-((1s,4s)-4-(hydroxymethyl)cyclohexyl)-1H-benzo[d]imidazol-2-yl)-3-(trifluoromethyl)benzamide (GT-05325)

With reference to the method of Scheme T-3, the target compound (GT-05325) was obtained (yellow solid, 42 mg, yield 54.69%). ¹H NMR (400 MHz, DMSO) δ 12.79 (s, 1H), 10.86 (d, J=3.2 Hz, 2H), 10.11 (s, 1H), 9.67 (s, 1H), 8.50 (d, J=7.8 Hz, 1H), 8.45 (s, 1H), 7.90 (d, J=7.6 Hz, 1H), 7.74 (t, J=6.5 Hz, 2H), 7.53 (d, J=8.9 Hz, 1H), 7.43-7.36 (m, 3H), 7.23 (d, J=2.0 Hz, 1H), 7.05 (d, J=8.9 Hz, 1H), 4.71 (s, 1H), 3.81-3.66 (m, 10H), 3.24 (d, J=8.2 Hz, 4H), 2.99 (dd, J=22.6, 12.9 Hz, 3H), 2.83 (t, J=6.8 Hz, 3H), 2.10 (s, 1H), 2.04-1.92 (m, 5H), 1.85 (s, 1H), 1.75-1.60 (m, 5H), 1.56-1.42 (m, 2H). LCMS (ESI) calcd. for C₄₄H₄₉F₃N₉O₆⁺ [M+H]⁺: 856.38, found, 856.4.

### Example 65: Preparation of 6-(5-cyano-1H-pyrrolo[2,3-b]pyridin-1-yl)-N-(1-((2-(2,4-dioxotetrahydropyrimidin-1(2H)-yl)-1,3-dioxoisoindolin-5-yl)methyl)piperidin-4-yl)-4-((isopropyl)amino) nicotinamide (GT-05326)

With reference to the method of Scheme T-1, the target compound (GT-05326) was obtained (yellow solid, 17 mg, yield 25.41%). ¹H NMR (400 MHz, DMSO) δ 11.04 (s, 1H), 10.93 (s, 1H), 8.87-8.80 (m, 1H), 8.79-8.63 (m, 3H), 8.52 (t, J=5.5 Hz, 1H), 8.34 (s, 1H), 8.19 (d, J=7.7 Hz, 1H), 8.15-8.04 (m, 2H), 6.92 (dd, J=10.7, 3.9 Hz, 1H), 4.56 (d, J=20.7 Hz, 2H), 4.00 (s, 1H), 3.83 (t, J=6.7 Hz, 3H), 3.41 (s, 2H), 3.13 (t, J=14.5 Hz, 2H), 2.87 (t, J=6.7 Hz, 2H), 2.03 (t, J=24.2 Hz, 4H), 1.29 (d, J=6.3 Hz, 6H). LCMS (ESI) calcd. for C₃₅H₃₅N₁₀O₅⁺ [M+H]⁺: 675.28, found, 675.3.

### Example 66: Preparation of 7-cyclopentyl-2-((5-(4-(((1S,4S)-5-((2-(2,4-dioxotetrahydropyrimidin-1(2H)-yl)-1,3-dioxoisoindolin-5-yl)methyl)-2,5-diazabicyclo[2.2.1]heptan-2-yl)methyl)piperidin-1-yl)pyridin-2-yl)amino)-N,N-dimethyl-7H-pyrrolo[2,3-d]pyrimidine-6-carboxamide (GT-05197)

With reference to the method of Scheme T-3, the target compound (GT-05197) was obtained (yellow solid, 57 mg, yield 63%). ¹H NMR (400 MHz, DMSO) δ 11.48 (s, 1H), 10.84 (s, 1H), 8.99 (s, 1H), 8.13 (d, J=9.8 Hz, 1H), 7.91 (s, 1H), 7.76 (d, J=8.4 Hz, 1H), 7.65-7.48 (m, 1H), 7.20 (s, 1H), 7.02 (s, 1H), 6.82 (s, 1H), 5.02 (s, 1H), 4.88-4.74 (m, 1H), 4.58 (s, 1H), 4.09 (s, 1H), 3.80 (d, J=6.6 Hz, 3H), 3.75-3.66 (m, 4H), 3.17 (s, 2H), 3.06 (s, 7H), 2.84 (t, J=7.0 Hz, 2H), 2.78 (d, J=11.4 Hz, 2H), 2.44 (s, 1H), 2.34-2.26 (m, 2H), 2.22 (s, 1H), 2.10-1.93 (m, 7H), 1.65 (d, J=5.6 Hz, 2H), 1.38 (d, J=10.2 Hz, 2H). LCMS (ESI) calcd. for C₄₂H₄₉N₁₂O₅⁺ [M+H]⁺: 801.39, found, 801.4.

### Example 67: Preparation of 7-cyclopentyl-2-((5-(4-(((1R,4R)-5-((2-(2,4-dioxotetrahydropyrimidin-1(2H)-yl)-1,3-dioxoisoindolin-5-yl)methyl)-2,5-diazabicyclo[2.2.1]heptan-2-yl)methyl)piperidin-1-yl)pyridin-2-yl)amino)-N,N-dimethyl-7H-pyrrolo[2,3-d]pyrimidine-6-carboxamide (GT-05198)

With reference to the method of Scheme T-3, the target compound (GT-05198) was obtained (yellow solid, 45 mg, yield 47.77%). ¹H NMR (400 MHz, DMSO) δ 11.51 (s, 1H), 10.84 (s, 1H), 9.00 (s, 1H), 8.14 (d, J=9.5 Hz, 1H), 7.92 (s, 1H), 7.76 (d, J=8.3 Hz, 1H), 7.64-7.50 (m, 1H), 7.21 (d, J=20.1 Hz, 1H), 7.02 (s, 1H), 6.82 (s, 1H), 5.02 (s, 1H), 4.86-4.74 (m, 1H), 4.58 (s, 1H), 3.79 (t, J=6.7 Hz, 3H), 3.75-3.66 (m, 4H), 3.20-3.11 (m, 2H), 3.06 (s, 7H), 2.84 (t, J=7.0 Hz, 2H), 2.78 (d, J=8.4 Hz, 2H), 2.45 (d, J=11.2 Hz, 1H), 2.32 (d, J=11.4 Hz, 2H), 2.24 (d, J=15.5 Hz, 1H), 2.09-1.94 (m, 7H), 1.73-1.60 (m, 2H), 1.38 (d, J=10.9 Hz, 2H). LCMS (ESI) calcd, for C₄₂H₄₉N₁₂O₅⁺ [M+H]⁺: 801.39, found, 801.4.

### Example 68: Preparation of 7-cyclopentyl-2-((5-(4-((4-(2-(2,4-dioxotetrahydropyrimidin-1(2H)-yl)-6-fluoro-1,3-dioxoisoindolin-5-yl)piperazin-1-yl)methyl)piperidin-1-yl)pyridin-2-yl)amino)-N,N-dimethyl-7H-pyrrolo[2,3-d]pyrimidine-6-carboxamide (GT-05199)

With reference to the method of Scheme T-3, the target compound (GT-05199) was obtained (yellow solid, 14 mg, yield 10.76%). ¹H NMR (400 MHz, DMSO) δ 11.56 (s, 1H), 11.00 (s, 1H), 10.90 (s, 1H), 9.01 (d, J=2.1 Hz, 1H), 8.17 (d, J=9.4 Hz, 1H), 7.96-7.88 (m, 2H), 7.69-7.63 (m, 1H), 7.59 (d, J=9.7 Hz, 1H), 6.84 (s, 1H), 4.85-4.79 (m, 1H), 3.80 (d, J=6.8 Hz, 3H), 3.72 (d, J=12.1 Hz, 4H), 3.65 (s, 4H), 3.14 (s, 2H), 3.07 (s, 7H), 2.84 (d, J=7.1 Hz, 2H), 2.32 (d, J=10.2 Hz, 3H), 2.01 (dd, J=20.5, 8.5 Hz, 7H), 1.66 (s, 3H), 1.42 (d, J=9.9 Hz, 2H). LCMS (ESI) calcd, for C₄₁H₄₈FN₁₂O₅⁺ [M+H]⁺: 807.38, found, 807.4.

### Example 69: Preparation of 7-cyclopentyl-2-((5-(4-((8-(2-(2,4-dioxotetrahydropyrimidin-1(2H)-yl)-1,3-dioxoisoindolin-5-yl)-3,8-diazabicyclo[3.2.1]octan-3-yl)methyl)piperidin-1-yl)pyridin-2-yl)amino)-N,N-dimethyl-7H-pyrrolo[2,3-d]pyrimidine-6-carboxamide (GT-05200)

With reference to the method of Scheme T-3, the target compound (GT-05200) was obtained (yellow solid, 23 mg, yield 20.01%). ¹H NMR (400 MHz, DMSO) δ 11.45 (s, 1H), 10.86 (s, 1H), 10.30 (s, 1H), 8.99 (s, 1H), 8.10 (d, J=7.4 Hz, 1H), 7.91-7.79 (m, 2H), 7.56 (d, J=9.7 Hz, 1H), 7.47 (s, 1H), 7.33 (d, J=8.5 Hz, 1H), 6.82 (s, 1H), 4.86-4.77 (m, 3H), 3.80 (t, J=6.8 Hz, 2H), 3.64 (d, J=11.6 Hz, 3H), 3.06 (s, 7H), 2.96 (d, J=6.6 Hz, 2H), 2.85 (t, J=7.0 Hz, 2H), 2.75 (t, J=10.7 Hz, 2H), 2.40 (d, J=5.8 Hz, 2H), 2.32 (d, J=11.1 Hz, 3H), 2.03 (dd, J=38.6, 17.2 Hz, 7H), 1.90 (d, J=12.9 Hz, 2H), 1.65 (d, J=5.9 Hz, 2H), 1.35 (d, J=10.0 Hz, 2H). LCMS (ESI) calcd, for C₄₃H₅₁N₁₂O₅⁺ [M+H]⁺: 815.41, found, 815.4.

### Example 70: Preparation of 7-cyclopentyl-2-((5-(4-((6-(2-(2,4-dioxotetrahydropyrimidin-1(2H)-yl)-1,3-dioxoisoindolin-5-yl)-3,6-diazabicyclo[3.1.1]heptan-3-yl)methyl)piperidin-1-yl)pyridin-2-yl)amino)-N,N-dimethyl-7H-pyrrolo[2,3-d]pyrimidine-6-carboxamide (GT-05201)

With reference to the method of Scheme T-3, the target compound (GT-05201) was obtained (yellow solid, 24 mg, yield 21.23%). ¹H NMR (400 MHz, DMSO) δ 11.37 (d, J=76.1 Hz, 2H), 10.85 (s, 1H), 8.99 (s, 1H), 8.10 (d, J=9.6 Hz, 1H), 7.97-7.76 (m, 2H), 7.56 (d, J=9.7 Hz, 1H), 7.23 (s, 1H), 7.02 (d, J=8.3 Hz, 1H), 6.82 (s, 1H), 4.87-4.76 (m, 1H), 4.69 (s, 1H), 3.77 (dd, J=13.9, 7.2 Hz, 4H), 3.63 (d, J=12.3 Hz, 2H), 3.29-3.18 (m, 2H), 3.06 (s, 7H), 2.92-2.74 (m, 6H), 2.68 (t, J=11.5 Hz, 2H), 2.32 (d, J=10.6 Hz, 2H), 2.07-1.88 (m, 7H), 1.65 (d, J=5.8 Hz, 2H), 1.33 (d, J=10.3 Hz, 2H). LCMS (ESI) calcd, for C₄₂H₄₉N₁₂O₅⁺ [M+H]⁺: 801.39, found, 801.4.

### Example 71: Preparation of 7-cyclopentyl-2-((5-(4-((4-(2-(2,4-dioxotetrahydropyrimidin-1(2H)-yl)-1,3-dioxoisoindolin-4-yl)piperazin-1-yl)methyl)piperidin-1-yl)pyridin-2-yl)amino)-N,N-dimethyl-7H-pyrrolo[2,3-d]pyrimidine-6-carboxamide (GT-05202)

With reference to the method of Scheme T-3, the target compound (GT-05202) was obtained (yellow solid, 27 mg, yield 24.23%). ¹H NMR (400 MHz, DMSO) δ 11.54 (s, 1H), 11.16 (s, 1H), 10.88 (s, 1H), 9.01 (s, 1H), 8.17 (d, J=9.5 Hz, 1H), 7.95 (s, 1H), 7.83 (t, J=7.8 Hz, 1H), 7.60 (d, J=9.5 Hz, 1H), 7.55-7.47 (m, 2H), 6.83 (s, 1H), 4.86-4.74 (m, 1H), 3.88 (d, J=11.2 Hz, 1H), 3.80 (t, J=6.8 Hz, 3H), 3.69 (s, 4H), 3.24 (d, J=6.6 Hz, 2H), 3.15 (s, 3H), 3.06 (s, 7H), 2.87-2.81 (m, 3H), 2.32 (d, J=6.7 Hz, 3H), 2.02 (dd, J=29.1, 9.4 Hz, 7H), 1.66 (s, 3H), 1.42 (d, J=11.3 Hz, 2H). LCMS (ESI) calcd, for C₄₁H₄₉N₁₂O₅⁺ [M+H]⁺: 789.39, found, 789.4.

### Example 72: Preparation of 7-cyclopentyl-2-((5-(4-((4-(2-(2,4-dioxotetrahydropyrimidin-1(2H)-yl)-4-fluoro-1,3-dioxoisoindolin-5-yl)piperazin-1-yl)methyl)piperidin-1-yl)pyridin-2-yl)amino)-N,N-dimethyl-7H-pyrrolo[2,3-d]pyrimidine-6-carboxamide (GT-05203)

With reference to the method of Scheme T-3, the target compound (GT-05203) was obtained (yellow solid, 48 mg, yield 52.70%). ¹H NMR (400 MHz, DMSO) δ 11.50 (s, 1H), 11.01 (s, 1H), 10.90 (s, 1H), 9.00 (s, 1H), 8.15 (d, J=9.6 Hz, 1H), 7.92 (s, 1H), 7.77 (d, J=8.1 Hz, 1H), 7.56 (dd, J=16.8, 8.8 Hz, 2H), 6.83 (s, 1H), 4.91-4.74 (m, 1H), 3.80 (t, J=6.7 Hz, 2H), 3.71 (d, J=12.8 Hz, 3H), 3.62 (d, J=8.9 Hz, 4H), 3.29-3.22 (m, 4H), 3.13 (s, 2H), 3.06 (s, 6H), 2.83 (dd, J=12.4, 5.6 Hz, 3H), 2.32 (d, J=8.6 Hz, 2H), 2.08 (s, 1H), 2.09-1.96 (m, 6H), 1.66 (d, J=5.8 Hz, 2H), 1.41 (d, J=11.1 Hz, 2H). LCMS (ESI) calcd, for C₄₁H₄₈FN₁₂O₅⁺ [M+H]⁺: 807.38, found, 807.4.

### Example 73: Preparation of 7-cyclopentyl-2-((5-(4-((4-(2-(2,4-dioxotetrahydropyrimidin-1(2H)-yl)-7-fluoro-1,3-dioxoisoindolin-5-yl)piperazin-1-yl)methyl)piperidin-1-yl)pyridin-2-yl)amino)-N,N-dimethyl-7H-pyrrolo[2,3-d]pyrimidine-6-carboxamide (GT-05204)

With reference to the method of Scheme T-3, the target compound (GT-05204) was obtained (yellow solid, 31 mg, yield 34.03%). ¹H NMR (400 MHz, DMSO) δ 11.47 (s, 1H), 10.96 (s, 1H), 10.88 (s, 1H), 9.00 (s, 1H), 8.14 (d, J=9.7 Hz, 1H), 7.92 (s, 1H), 7.58 (d, J=9.7 Hz, 1H), 7.45 (s, 1H), 7.26 (d, J=12.9 Hz, 1H), 6.82 (s, 1H), 4.86-4.76 (m, 1H), 4.28 (s, 2H), 3.81-3.76 (m, 2H), 3.70 (d, J=12.6 Hz, 4H), 3.61 (d, J=11.1 Hz, 3H), 3.17 (s, 1H), 3.10 (s, 2H), 3.06 (s, 6H), 2.82 (dd, J=18.5, 11.3 Hz, 4H), 2.33 (s, 2H), 2.13-1.94 (m, 7H), 1.66 (d, J=6.0 Hz, 2H), 1.38 (d, J=11.1 Hz, 2H). LCMS (ESI) calcd, for C₄₁H₄₈FN₁₂O₅⁺ [M+H]⁺: 807.38, found, 807.4.

### Example 74: Preparation of 7-cyclopentyl-2-((5-(4-((4-(1-(2-(2,4-dioxotetrahydropyrimidin-1(2H)-yl)-1,3-dioxoisoindolin-5-yl)piperidin-4-yl)piperazin-1-yl)methyl)piperidin-1-yl)pyridin-2-yl)amino)-N,N-dimethyl-7H-pyrrolo[2,3-d]pyrimidine-6-carboxamide (GT-05205)

With reference to the method of Scheme T-3, the target compound (GT-05205) was obtained (yellow solid, 25 mg, yield 25.48%). ¹H NMR (400 MHz, DMSO) δ 11.45 (s, 1H), 10.84 (s, 1H), 8.99 (s, 1H), 8.13 (d, J=9.8 Hz, 1H), 7.91 (s, 1H), 7.76 (d, J=8.6 Hz, 1H), 7.58 (d, J=9.5 Hz, 1H), 7.48 (s, 1H), 7.37 (d, J=8.8 Hz, 1H), 6.82 (s, 1H), 4.88-4.75 (m, 1H), 4.29 (d, J=11.9 Hz, 2H), 3.79 (t, J=6.8 Hz, 3H), 3.69 (d, J=11.9 Hz, 5H), 3.31-3.28 (m, 4H), 3.17 (s, 1H), 3.06 (s, 8H), 3.00 (d, J=11.9 Hz, 2H), 2.83 (t, J=7.0 Hz, 3H), 2.78 (s, 1H), 2.33 (s, 2H), 2.18 (s, 2H), 2.00 (d, J=21.0 Hz, 7H), 1.76 (s, 2H), 1.65 (d, J=5.7 Hz, 2H), 1.36 (d, J=11.9 Hz, 2H). LCMS (ESI) calcd, for C₄₆H₅₈N₁₃O₅⁺ [M+H]⁺: 872.47, found, 872.5.

### Example 75: Preparation of 7-cyclopentyl-2-((5-(4-((4-(1-(2-(2,4-dioxotetrahydropyrimidin-1(2H)-yl)-1,3-dioxoisoindolin-5-yl)-3,3-difluoropiperidin-4-yl)piperazin-1-yl)methyl)piperidin-1-yl)pyridin-2-yl)amino)-N,N-dimethyl-7H-pyrrolo[2,3-d]pyrimidine-6-carboxamide (GT-05206)

With reference to the method of Scheme T-3, the target compound (GT-05206) was obtained (yellow solid, 32 mg, yield 31.37%). ¹H NMR (400 MHz, DMSO) δ 11.49 (s, 1H), 10.85 (s, 1H), 10.24 (s, 1H), 9.00 (s, 1H), 8.24-8.00 (m, 1H), 7.91 (s, 1H), 7.75 (d, J=8.3 Hz, 1H), 7.58 (d, J=9.5 Hz, 1H), 7.51 (d, J=2.8 Hz, 1H), 7.42 (d, J=8.8 Hz, 1H), 6.82 (s, 1H), 4.81 (p, J=8.9 Hz, 1H), 4.48 (s, 1H), 4.26 (d, J=12.5 Hz, 1H), 3.79 (t, J=6.8 Hz, 2H), 3.69 (d, J=11.5 Hz, 2H), 3.24-3.08 (m, 6H), 3.05 (d, J=8.2 Hz, 10H), 2.97 (d, J=10.8 Hz, 2H), 2.83 (t, J=7.0 Hz, 2H), 2.76 (d, J=10.8 Hz, 1H), 2.35-2.25 (m, 2H), 1.98 (t, J=15.4 Hz, 7H), 1.88 (s, 2H), 1.70-1.60 (m, 2H), 1.37 (d, J=10.7 Hz, 2H). LCMS (ESI) calcd, for C₄₆H₅₆F₂N₁₃O₅⁺ [M+H]⁺: 908.45, found, 908.5.

### Example 76: Preparation of N-((1r,4r)-4-(3-chloro-4-cyanophenoxy)cyclohexyl)-6-(4-((4-(2-(2,4-dioxotetrahydropyrimidin-1(2H)-yl)-1,3-dioxoisoindolin-5-yl)piperazin-1-yl)methyl)piperidin-1-yl)pyridazine-3-carboxamide (GT-05225)

With reference to the method of Scheme T-3, the target compound (GT-05225) was obtained (yellow solid, 26 mg, yield 29.21%). ¹H NMR (400 MHz, DMSO) δ 10.86 (s, 1H), 10.56 (s, 1H), 8.60 (d, J=8.3 Hz, 1H), 7.91-7.75 (m, 3H), 7.56 (d, J=2.1 Hz, 1H), 7.45-7.34 (m, 3H), 7.14 (dd, J=8.8, 2.4 Hz, 1H), 4.53 (dd, J=20.5, 11.3 Hz, 3H), 4.23 (d, J=13.0 Hz, 2H), 3.92-3.84 (m, 1H), 3.80 (t, J=7.0 Hz, 2H), 3.58 (dd, J=29.3, 13.5 Hz, 5H), 3.17 (t, J=8.4 Hz, 3H), 3.08 (t, J=11.9 Hz, 4H), 2.84 (t, J=7.1 Hz, 2H), 2.26 (s, 1H), 2.11 (d, J=9.9 Hz, 2H), 1.93 (dd, J=24.9, 11.1 Hz, 4H), 1.71-1.60 (m, 2H), 1.60-1.44 (m, 3H), 1.37-1.19 (m, 3H), 0.94 (t, J=7.3 Hz, 1H). LCMS (ESI) calcd, for C₄₀H₄₄ClN₁₀O₆⁺ [M+H]⁺: 795.31, found, 795.4.

### Example 77: Preparation of N-((1r,4r)-4-(3-chloro-4-cyanophenoxy)cyclohexyl)-6-(4-(((1S,4S)-5-(2-(2,4-dioxotetrahydropyrimidin-1(2H)-yl)-1,3-dioxoisoindolin-5-yl)-2,5-diazabicyclo[2.2.1]heptan-2-yl)methyl)piperidin-1-yl)pyridazine-3-carboxamide (GT-05226)

With reference to the method of Scheme T-3, the target compound (GT-05226) was obtained (yellow solid, 23 mg, yield 25.48%). ¹H NMR (400 MHz, DMSO) δ 10.85 (d, J=3.5 Hz, 1H), 8.58 (d, J=8.2 Hz, 1H), 7.86 (d, J=8.9 Hz, 1H), 7.82 (t, J=4.8 Hz, 1H), 7.77 (d, J=8.4 Hz, 1H), 7.44-7.36 (m, 2H), 7.21 (d, J=17.4 Hz, 1H), 7.13 (dd, J=8.8, 2.4 Hz, 1H), 7.03 (s, 1H), 4.99 (d, J=21.3 Hz, 1H), 4.51 (dd, J=24.4, 10.5 Hz, 3H), 3.96 (d, J=11.3 Hz, 1H), 3.84 (s, 1H), 3.82-3.77 (m, 2H), 3.74 (d, J=9.5 Hz, 2H), 3.30 (d, J=9.5 Hz, 2H), 3.17 (s, 1H), 3.03 (s, 3H), 2.84 (t, J=6.8 Hz, 2H), 2.43 (d, J=10.4 Hz, 1H), 2.20 (d, J=11.6 Hz, 1H), 2.10 (d, J=10.7 Hz, 3H), 1.99-1.85 (m, 4H), 1.67-1.57 (m, 2H), 1.51 (dd, J=22.7, 10.3 Hz, 2H), 1.23 (s, 2H). LCMS (ESI) calcd, for C₄₁H₄₄ClN₁₀O₆⁺ [M+H]⁺: 807.31, found, 807.4.

### Example 78: Preparation of N-((1r,4r)-4-(3-chloro-4-cyanophenoxy)cyclohexyl)-6-(4-(((1R,4R)-5-(2-(2,4-dioxotetrahydropyrimidin-1(2H)-yl)-1,3-dioxoisoindolin-5-yl)-2,5-diazabicyclo[2.2.1]heptan-2-yl)methyl)piperidin-1-yl)pyridazine-3-carboxamide (GT-05227)

With reference to the method of Scheme T-3, the target compound (GT-05227) was obtained (yellow solid, 27 mg, yield 29.91%). ¹H NMR (400 MHz, DMSO) δ 10.85 (d, J=3.6 Hz, 1H), 8.58 (d, J=8.2 Hz, 1H), 7.86 (d, J=8.8 Hz, 1H), 7.82 (t, J=4.8 Hz, 1H), 7.77 (d, J=8.4 Hz, 1H), 7.43-7.36 (m, 2H), 7.20 (d, J=21.2 Hz, 1H), 7.13 (dd, J=8.8, 2.4 Hz, 1H), 7.03 (s, 1H), 4.99 (d, J=21.7 Hz, 1H), 4.52 (dd, J=22.5, 11.8 Hz, 3H), 3.90 (dd, J=35.1, 11.1 Hz, 2H), 3.81-3.65 (m, 4H), 3.30 (d, J=11.0 Hz, 2H), 3.19-3.08 (m, 2H), 3.03 (s, 3H), 2.84 (t, J=7.1 Hz, 2H), 2.42 (t, J=9.5 Hz, 1H), 2.28-2.13 (m, 2H), 2.10 (d, J=10.6 Hz, 2H), 1.98-1.85 (m, 4H), 1.70-1.59 (m, 2H), 1.58-1.49 (m, 2H), 1.28 (dd, J=21.0, 13.7 Hz, 2H). LCMS (ESI) calcd. for C₄₁H₄₄ClN₁₀O₆⁺ [M+H]⁺: 807.31, found, 807.4.

### Example 79: Preparation of N-((1r,4r)-4-(3-chloro-4-cyanophenoxy)cyclohexyl)-6-(4-((4-(2-(2,4-dioxotetrahydropyrimidin-1(2H)-yl)-6-fluoro-1,3-dioxoisoindolin-5-yl)piperazin-1-yl)methyl)piperidin-1-yl)pyridazine-3-carboxamide (GT-05228)

With reference to the method of Scheme T-3, the target compound (GT-05228) was obtained (yellow solid, 20 mg, yield 22.00%). ¹H NMR (400 MHz, DMSO) δ 10.90 (s, 1H), 10.48 (s, 1H), 8.60 (d, J=8.2 Hz, 1H), 7.91 (d, J=11.3 Hz, 1H), 7.85 (dd, J=9.2, 3.9 Hz, 2H), 7.67 (d, J=7.5 Hz, 1H), 7.43 (d, J=9.7 Hz, 1H), 7.39 (d, J=2.4 Hz, 1H), 7.14 (dd, J=8.8, 2.4 Hz, 1H), 4.61-4.46 (m, 3H), 3.82 (dd, J=21.2, 14.2 Hz, 5H), 3.66-3.54 (m, 4H), 3.24 (s, 2H), 3.13-3.04 (m, 4H), 2.85 (t, J=6.8 Hz, 2H), 2.25 (s, 1H), 2.11 (d, J=10.0 Hz, 2H), 1.99-1.87 (m, 4H), 1.64 (dd, J=23.4, 10.4 Hz, 2H), 1.59-1.49 (m, 2H), 1.33-1.22 (m, 2H). LCMS (ESI) calcd, for C₄₀H₄₃ClFN₁₀O₆⁺ [M+H]⁺: 813.30, found, 813.3.

### Example 80: Preparation of N-((1r,4r)-4-(3-chloro-4-cyanophenoxy)cyclohexyl)-6-(4-((8-(2-(2,4-dioxotetrahydropyrimidin-1(2H)-yl)-1,3-dioxoisoindolin-5-yl)-3,8-diazabicyclo[3.2.1]octan-3-yl)methyl)piperidin-1-yl)pyridazine-3-carboxamide (GT-05229)

With reference to the method of Scheme T-3, the target compound (GT-05229) was obtained (yellow solid, 26 mg, yield 28.33%). ¹H NMR (400 MHz, DMSO) § 10.86 (s, 1H), 10.08 (s, 1H), 8.58 (d, J=8.2 Hz, 1H), 7.84 (dd, J=15.2, 9.2 Hz, 3H), 7.46 (s, 1H), 7.41-7.35 (m, 2H), 7.32 (d, J=8.9 Hz, 1H), 7.13 (dd, J=8.8, 2.4 Hz, 1H), 4.81 (s, 2H), 4.53 (s, 1H), 4.43 (d, J=13.3 Hz, 2H), 3.85 (d, J=8.1 Hz, 1H), 3.79 (t, J=6.9 Hz, 2H), 3.18 (d, J=6.0 Hz, 1H), 3.04 (dd, J=26.0, 12.5 Hz, 4H), 2.92 (s, 2H), 2.84 (t, J=7.3 Hz, 2H), 2.35 (d, J=8.2 Hz, 2H), 2.18 (s, 1H), 2.11 (s, 3H), 1.87 (t, J=12.7 Hz, 4H), 1.70-1.60 (m, 2H), 1.58-1.48 (m, 3H), 1.35-1.28 (m, 1H), 1.22 (d, J=12.0 Hz, 2H). LCMS (ESI) calcd, for C₄₂H₄₆ClN₁₀O₆⁺ [M+H]⁺: 821.33, found, 821.4.

### Example 81: Preparation of N-((1r,4r)-4-(3-chloro-4-cyanophenoxy)cyclohexyl)-6-(4-((4-(2-(2,4-dioxotetrahydropyrimidin-1(2H)-yl)-1,3-dioxoisoindolin-4-yl)piperazin-1-yl)methyl)piperidin-1-yl)pyridazine-3-carboxamide (GT-05230)

With reference to the method of Scheme T-3, the target compound (GT-05230) was obtained (yellow solid, 32 mg, yield 35.96%). ¹H NMR (400 MHz, DMSO) δ 10.89 (s, 1H), 10.58 (s, 1H), 8.60 (d, J=8.2 Hz, 1H), 7.88-7.80 (m, 3H), 7.50 (dd, J=11.1, 7.9 Hz, 2H), 7.40 (dd, J=11.7, 6.0 Hz, 2H), 7.14 (dd, J=8.8, 2.4 Hz, 1H), 4.51 (d, J=13.7 Hz, 3H), 3.89 (d, J=11.4 Hz, 2H), 3.80 (t, J=6.8 Hz, 3H), 3.69 (s, 2H), 3.61-3.54 (m, 2H), 3.18 (d, J=6.6 Hz, 2H), 3.06 (d, J=11.4 Hz, 1H), 2.88-2.78 (m, 2H), 2.28 (s, 1H), 2.11 (d, J=9.7 Hz, 2H), 1.98 (d, J=13.1 Hz, 2H), 1.90 (d, J=10.7 Hz, 2H), 1.65 (d, J=13.2 Hz, 2H), 1.55 (dd, J=24.7, 11.2 Hz, 4H), 1.29 (dd, J=15.2, 8.1 Hz, 3H). LCMS (ESI) calcd, for C₄₀H₄₄ClN₁₀O₆⁺ [M+H]⁺: 795.31, found, 795.3.

### Example 82: Preparation of N-((1r,4r)-4-(3-chloro-4-cyanophenoxy)cyclohexyl)-6-(4-((6-(2-(2,4-dioxotetrahydropyrimidin-1(2H)-yl)-1,3-dioxoisoindolin-5-yl)-3,6-diazabicyclo[3.1.1]heptan-3-yl)methyl)piperidin-1-yl)pyridazine-3-carboxamide (GT-05231)

With reference to the method of Scheme T-3, the target compound (GT-05231) was obtained (yellow solid, 22 mg, yield 24.37%). ¹H NMR (400 MHz, DMSO) δ 10.86 (s, 1H), 10.61 (s, 1H), 8.57 (d, J=8.3 Hz, 1H), 7.83 (dd, J=17.7, 9.3 Hz, 3H), 7.37 (dd, J=9.7, 6.1 Hz, 2H), 7.23 (s, 1H), 7.13 (dd, J=8.8, 2.4 Hz, 1H), 7.01 (d, J=8.4 Hz, 1H), 4.68 (s, 2H), 4.53 (s, 1H), 4.43 (s, 2H), 3.81 (s, 2H), 3.78 (t, J=6.7 Hz, 4H), 2.95 (t, J=11.9 Hz, 1H), 2.84 (t, J=6.9 Hz, 4H), 2.60 (d, J=10.0 Hz, 1H), 2.10 (d, J=9.3 Hz, 3H), 1.88 (s, 4H), 1.63 (dd, J=23.9, 11.0 Hz, 2H), 1.57-1.48 (m, 2H), 1.18 (d, J=9.4 Hz, 2H), 0.94 (t, J=7.3 Hz, 1H). LCMS (ESI) calcd, for C₄₁H₄₄ClN₁₀O₆⁺ [M+H]⁺: 807.31, found, 807.3.

### Example 83: Preparation of N-((1r,4r)-4-(3-chloro-4-cyanophenoxy)cyclohexyl)-6-(4-((4-(2-(2,4-dioxotetrahydropyrimidin-1(2H)-yl)-4-fluoro-1,3-dioxoisoindolin-5-yl)piperazin-1-yl)methyl)piperidin-1-yl)pyridazine-3-carboxamide (GT-05232)

With reference to the method of Scheme T-3, the target compound (GT-05232) was obtained (yellow solid, 24 mg, yield 26.40%). ¹H NMR (400 MHz, DMSO) δ 10.91 (s, 1H), 8.60 (d, J=8.3 Hz, 1H), 7.85 (dd, J=9.2, 6.5 Hz, 2H), 7.77 (d, J=8.1 Hz, 1H), 7.55 (t, J=7.9 Hz, 1H), 7.40 (dd, J=10.9, 6.1 Hz, 2H), 7.14 (dd, J=8.8, 2.4 Hz, 1H), 4.51 (d, J=13.4 Hz, 3H), 3.86 (d, J=8.3 Hz, 1H), 3.79 (t, J=6.8 Hz, 4H), 3.63 (s, 3H), 3.12-3.05 (m, 4H), 2.84 (t, J=6.8 Hz, 2H), 2.26 (s, 1H), 2.11 (d, J=9.5 Hz, 2H), 1.93 (dd, J=20.6, 13.9 Hz, 4H), 1.70-1.61 (m, 2H), 1.55 (dd, J=26.7, 12.9 Hz, 3H), 1.29 (dd, J=16.4, 9.2 Hz, 3H), 0.93 (d, J=7.4 Hz, 1H). LCMS (ESI) calcd, for C₄₀H₄₃ClFN₁₀O₆⁺ [M+H]⁺: 813.30, found, 812.3.

### Example 84: Preparation of N-((1r,4r)-4-(3-chloro-4-cyanophenoxy)cyclohexyl)-6-(4-((4-(2-(2,4-dioxotetrahydropyrimidin-1(2H)-yl)-7-fluoro-1,3-dioxoisoindolin-5-yl)piperazin-1-yl)methyl)piperidin-1-yl)pyridazine-3-carboxamide (GT-05233)

With reference to the method of Scheme T-3, the target compound (GT-05233) was obtained (yellow solid, 29 mg, yield 31.90%). ¹H NMR (400 MHz, DMSO) δ 10.89 (s, 1H), 10.57 (s, 1H), 8.59 (d, J=8.2 Hz, 1H), 7.85 (dd, J=9.2, 6.2 Hz, 2H), 7.43 (dd, J=12.9, 5.6 Hz, 2H), 7.39 (d, J=2.4 Hz, 1H), 7.26 (d, J=13.1 Hz, 1H), 7.13 (dd, J=8.8, 2.4 Hz, 1H), 4.56-4.46 (m, 3H), 4.28 (s, 2H), 3.86 (d, J=8.5 Hz, 1H), 3.79 (td, J=6.8, 1.9 Hz, 2H), 3.62 (s, 2H), 3.19-3.11 (m, 4H), 3.07 (d, J=10.4 Hz, 3H), 2.84 (t, J=7.4 Hz, 2H), 2.26 (s, 1H), 2.11 (d, J=9.6 Hz, 2H), 1.93 (dd, J=21.9, 13.0 Hz, 4H), 1.69-1.60 (m, 2H), 1.55 (dd, J=23.5, 13.4 Hz, 3H), 1.34-1.21 (m, 3H). LCMS (ESI) calcd, for C₄₀H₄₃ClFN₁₀O₆⁺ [M+H]⁺: 813.30, found, 813.3.

### Example 85: Preparation of N-((1r,4r)-4-(3-chloro-4-cyanophenoxy)cyclohexyl)-6-(4-((4-(1-(2-(2,4-dioxotetrahydropyrimidin-1(2H)-yl)-1,3-dioxoisoindolin-5-yl)-3,3-difluoropiperidin-4-yl)piperazin-1-yl)methyl)piperidin-1-yl)pyridazine-3-carboxamide (GT-05235)

With reference to the method of Scheme T-3, the target compound (GT-05235) was obtained (yellow solid, 17 mg, yield 16.09%). ¹H NMR (400 MHz, DMSO) δ 10.78 (s, 1H), 9.78 (s, 1H), 8.51 (d, J=8.2 Hz, 1H), 7.81-7.75 (m, 2H), 7.68 (d, J=8.5 Hz, 1H), 7.44 (d, J=2.9 Hz, 1H), 7.33 (dd, J=9.0, 6.0 Hz, 3H), 7.06 (dd, J=8.8, 2.4 Hz, 1H), 4.44 (dd, J=27.7, 11.3 Hz, 4H), 4.19 (d, J=12.8 Hz, 1H), 3.79 (d, J=8.0 Hz, 1H), 3.72 (t, J=6.8 Hz, 2H), 3.10 (t, J=8.4 Hz, 2H), 3.01-2.90 (m, 8H), 2.76 (t, J=6.9 Hz, 2H), 2.11 (s, 1H), 2.04 (d, J=10.1 Hz, 2H), 1.81 (s, 6H), 1.59-1.51 (m, 2H), 1.45 (dd, J=23.1, 10.8 Hz, 3H), 1.27-1.21 (m, 1H), 1.20-1.10 (m, 2H). LCMS (ESI) calcd, for C₄₅H₅₁ClF₂N₁₁O₆⁺ [M+H]⁺: 914.37, found, 914.3.

### Example 86: Preparation of 5-((1R,4R)-2-oxa-5-azabicyclo[2.2.1]heptan-5-yl)-N-(3-(difluoromethyl)-1-((1r,4R)-4-((4-(2-(2,4-dioxotetrahydropyrimidin-1(2H)-yl)-1,3-dioxoisoindolin-5-yl)piperazin-1-yl)methyl)cyclohexyl)-1H-pyrazol-4-yl)pyrazolo[1,5-a]pyrimidine-3-carboxamide (GT-05236)

With reference to the method of Scheme T-3, the target compound (GT-05236) was obtained (yellow solid, 12 mg, yield 17.23%). ¹H NMR (400 MHz, DMSO) δ 10.87 (s, 1H), 10.09 (s, 1H), 9.52 (d, J=6.7 Hz, 1H), 8.78 (d, J=7.7 Hz, 1H), 8.41 (d, J=4.3 Hz, 1H), 8.26 (d, J=5.5 Hz, 1H), 7.83 (d, J=8.5 Hz, 1H), 7.56 (s, 1H), 7.41 (d, J=6.7 Hz, 1H), 4.77 (d, J=19.6 Hz, 1H), 4.23 (d, J=12.6 Hz, 3H), 3.84-3.76 (m, 4H), 3.66-3.60 (m, 3H), 3.53-3.44 (m, 5H), 3.17 (s, 2H), 3.08 (s, 2H), 2.84 (t, J=6.9 Hz, 2H), 2.09 (d, J=11.3 Hz, 2H), 1.99 (t, J=13.1 Hz, 4H), 1.83 (dd, J=23.5, 12.2 Hz, 2H), 1.22 (d, J=12.7 Hz, 2H). LCMS (ESI) calcd. for C₃₉H₄₃F₂N₁₂O₆⁺ [M+H]⁺: 813.34, found, 813.4.

### Example 87: Preparation of 5-((1R,4R)-2-oxa-5-azabicyclo[2.2.1]heptan-5-yl)-N-(3-(difluoromethyl)-1-((1S,4R)-4-(((1S,4S)-5-(2-(2,4-dioxotetrahydropyrimidin-1(2H)-yl)-1,3-dioxoisoindolin-5-yl)-2,5-diazabicyclo[2.2.1]heptan-2-yl)methyl)cyclohexyl)-1H-pyrazol-4-yl)pyrazolo[1,5-a]pyrimidine-3-carboxamide (GT-05237)

With reference to the method of Scheme T-3, the target compound (GT-05237) was obtained (yellow solid, 12 mg, yield 16.99%). ¹H NMR (400 MHz, DMSO) δ 10.85 (s, 1H), 9.51 (d, J=6.7 Hz, 1H), 8.78 (d, J=7.8 Hz, 1H), 8.40 (d, J=3.8 Hz, 1H), 8.26 (d, J=5.5 Hz, 1H), 7.78 (d, J=8.3 Hz, 1H), 7.31-7.05 (m, 2H), 7.01 (d, J=21.2 Hz, 1H), 5.05 (d, J=20.6 Hz, 1H), 4.86-4.61 (m, 2H), 4.22 (s, 2H), 3.80 (dd, J=14.6, 9.5 Hz, 6H), 3.67-3.59 (m, 4H), 3.26 (s, 2H), 3.11 (s, 2H), 2.84 (t, J=6.8 Hz, 2H), 2.05 (s, 3H), 2.02-1.92 (m, 3H), 1.83 (s, 1H), 1.78 (d, J=12.9 Hz, 2H), 1.19 (s, 2H). LCMS (ESI) calcd, for C₄₀H₄₃F₂N₁₂O₆⁺ [M+H]⁺: 825.34, found, 825.3.

### Example 88: Preparation of 5-((1R,4R)-2-oxa-5-azabicyclo[2.2.1]heptan-5-yl)-N-(3-(difluoromethyl)-1-((1R,4R)-4-(((1R,4R)-5-(2-(2,4-dioxotetrahydropyrimidin-1(2H)-yl)-1,3-dioxoisoindolin-5-yl)-2,5-diazabicyclo[2.2.1]heptan-2-yl)methyl)cyclohexyl)-1H-pyrazol-4-yl)pyrazolo[1,5-a]pyrimidine-3-carboxamide (GT-05238)

With reference to the method of Scheme T-3, the target compound (GT-05238) was obtained (yellow solid, 16 mg, yield 22.65%). ¹H NMR (400 MHz, DMSO) δ 10.85 (s, 1H), 9.50 (d, J=6.1 Hz, 1H), 8.79 (d, J=7.7 Hz, 1H), 8.40 (d, J=3.9 Hz, 1H), 8.25 (d, J=5.5 Hz, 1H), 7.77 (d, J=8.4 Hz, 1H), 7.25 (s, 1H), 7.19-6.89 (m, 2H), 5.00 (d, J=22.9 Hz, 1H), 4.77 (d, J=20.2 Hz, 1H), 4.22 (s, 1H), 3.90-3.67 (m, 7H), 3.65-3.52 (m, 2H), 3.45 (d, J=10.1 Hz, 1H), 3.25 (s, 3H), 3.13 (d, J=28.4 Hz, 2H), 2.84 (t, J=6.8 Hz, 2H), 2.45 (d, J=10.9 Hz, 1H), 2.21 (d, J=10.5 Hz, 1H), 2.04 (d, J=8.6 Hz, 3H), 1.96 (d, J=15.9 Hz, 2H), 1.78 (dd, J=31.8, 18.8 Hz, 3H), 1.18 (s, 2H). LCMS (ESI) calcd, for C₄₀H₄₃F₂N₁₂O₆⁺ [M+H]⁺: 825.34, found, 825.3.

### Example 89: Preparation of 5-((1R,4R)-2-oxa-5-azabicyclo[2.2.1]heptan-5-yl)-N-(3-(difluoromethyl)-1-((1r,4R)-4-((4-(2-(2,4-dioxotetrahydropyrimidin-1(2H)-yl)-6-fluoro-1,3-dioxoisoindolin-5-yl)piperazin-1-yl)methyl)cyclohexyl)-1H-pyrazol-4-yl)pyrazolo[1,5-a]pyrimidine-3-carboxamide (GT-05239)

With reference to the method of Scheme T-3, the target compound (GT-05239) was obtained (yellow solid, 11 mg, yield 15.47%). ¹H NMR (400 MHz, DMSO) δ 10.89 (s, 1H), 9.51 (d, J=6.5 Hz, 1H), 8.79 (d, J=7.8 Hz, 1H), 8.41 (d, J=3.8 Hz, 1H), 8.26 (d, J=5.6 Hz, 1H), 7.91 (d, J=11.5 Hz, 1H), 7.65 (d, J=13.3 Hz, 1H), 7.16-6.98 (m, 1H), 4.77 (d, J=20.3 Hz, 1H), 4.25 (s, 1H), 3.88-3.74 (m, 6H), 3.68-3.59 (m, 4H), 3.51-3.43 (m, 2H), 3.25-3.20 (m, 2H), 3.17 (s, 1H), 3.09 (s, 2H), 2.85 (t, J=6.8 Hz, 2H), 2.76 (t, J=6.8 Hz, 1H), 2.07 (s, 2H), 2.00 (d, J=10.7 Hz, 4H), 1.82 (d, J=10.9 Hz, 2H), 1.23 (d, J=12.3 Hz, 2H). LCMS (ESI) calcd. for C₃₉H₄₂F₃N₁₂O₆⁺ [M+H]⁺: 831.33, found, 831.4.

### Example 90: Preparation of 5-((1R,4R)-2-oxa-5-azabicyclo[2.2.1]heptan-5-yl)-N-(3-(difluoromethyl)-1-((1r,4R)-4-((8-(2-(2,4-dioxotetrahydropyrimidin-1(2H)-yl)-1,3-dioxoisoindolin-5-yl)-3,8-diazabicyclo[3.2.1]octan-3-yl)methyl)cyclohexyl)-1H-pyrazol-4-yl)pyrazolo[1,5-a]pyrimidine-3-carboxamide (GT-05240)

With reference to the method of Scheme T-3, the target compound (GT-05240) was obtained (yellow solid, 29 mg, yield 40.40%). ¹H NMR (400 MHz, DMSO) δ 10.86 (s, 1H), 9.85 (s, 1H), 9.50 (d, J=6.5 Hz, 1H), 8.78 (d, J=7.8 Hz, 1H), 8.38 (d, J=4.1 Hz, 1H), 8.26 (d, J=5.6 Hz, 1H), 7.82 (d, J=8.5 Hz, 1H), 7.47 (s, 1H), 7.32 (d, J=8.0 Hz, 1H), 6.95 (dd, J=67.9, 28.9 Hz, 1H), 4.77 (d, J=19.8 Hz, 3H), 4.19 (s, 1H), 3.91-3.69 (m, 5H), 3.68-3.55 (m, 2H), 3.44 (s, 2H), 3.08 (d, J=10.5 Hz, 2H), 2.86 (dd, J=20.4, 13.3 Hz, 4H), 2.32 (d, J=7.1 Hz, 2H), 2.11 (s, 2H), 2.06-1.87 (m, 8H), 1.76 (d, J=12.5 Hz, 2H), 1.18 (d, J=12.1 Hz, 2H). LCMS (ESI) calcd, for C₄₁H₄₅F₂N₁₂O₆⁺ [M+H]⁺: 839.35, found, 839.3.

### Example 91: Preparation of 5-((1R,4R)-2-oxa-5-azabicyclo[2.2.1]heptan-5-yl)-N-(3-(difluoromethyl)-1-((1r,4R)-4-((4-(2-(2,4-dioxotetrahydropyrimidin-1(2H)-yl)-1,3-dioxoisoindolin-4-yl)piperazin-1-yl)methyl)cyclohexyl)-1H-pyrazol-4-yl)pyrazolo[1,5-a]pyrimidine-3-carboxamide (GT-05241)

With reference to the method of Scheme T-3, the target compound (GT-05241) was obtained (yellow solid, 13 mg, yield 18.67%). ¹H NMR (400 MHz, DMSO) δ 10.89 (s, 1H), 10.16 (s, 1H), 9.52 (d, J=6.6 Hz, 1H), 8.79 (d, J=7.7 Hz, 1H), 8.41 (d, J=3.8 Hz, 1H), 8.26 (d, J=5.6 Hz, 1H), 7.82 (d, J=7.5 Hz, 1H), 7.57-7.42 (m, 2H), 5.18 (d, J=79.9 Hz, 1H), 4.77 (d, J=19.6 Hz, 1H), 4.25 (s, 1H), 3.87 (s, 1H), 3.82-3.78 (m, 3H), 3.71 (d, J=19.6 Hz, 2H), 3.48 (dd, J=21.5, 11.2 Hz, 2H), 3.25-3.22 (m, 1H), 3.12 (s, 2H), 2.89-2.77 (m, 2H), 2.03 (dt, J=23.2, 14.0 Hz, 6H), 1.82 (d, J=12.2 Hz, 2H), 1.23 (d, J=12.6 Hz, 2H). LCMS (ESI) calcd. for C₃₉H₄₃F₂N₁₂O₆⁺ [M+H]⁺: 813.34, found, 813.3.

### Example 92: Preparation of 5-((1R,4R)-2-oxa-5-azabicyclo[2.2.1]heptan-5-yl)-N-(3-(difluoromethyl)-1-((1r,4R)-4-((6-(2-(2,4-dioxotetrahydropyrimidin-1(2H)-yl)-1,3-dioxoisoindolin-5-yl)-3,6-diazabicyclo[3.1.1]heptan-3-yl)methyl)cyclohexyl)-1H-pyrazol-4-yl)pyrazolo[1,5-a]pyrimidine-3-carboxamide (GT-05242)

With reference to the method of Scheme T-3, the target compound (GT-05242) was obtained (yellow solid, 22 mg, yield 31.15%). ¹H NMR (400 MHz, DMSO) δ 10.86 (s, 1H), 9.50 (d, J=6.4 Hz, 1H), 8.78 (d, J=7.7 Hz, 1H), 8.31 (dd, J=48.3, 4.5 Hz, 2H), 7.80 (t, J=27.0 Hz, 1H), 7.32-7.06 (m, 2H), 7.07-6.76 (m, 2H), 4.88-4.57 (m, 3H), 4.17 (s, 1H), 3.80 (t, J=11.7 Hz, 6H), 3.61 (d, J=13.3 Hz, 2H), 3.45 (d, J=10.9 Hz, 3H), 3.29-3.22 (m, 1H), 2.78 (t, J=35.3 Hz, 5H), 2.59 (d, J=9.7 Hz, 1H), 1.99 (dd, J=20.6, 12.0 Hz, 6H), 1.81 (s, 1H), 1.69 (d, J=13.0 Hz, 2H), 1.13 (d, J=12.2 Hz, 1H). LCMS (ESI) calcd, for C₄₀H₄₃F₂N₁₂O₆₊[M+H]⁺: 825.34, found, 825.3.

### Example 93: Preparation of 5-((1R,4R)-2-oxa-5-azabicyclo[2.2.1]heptan-5-yl)-N-(3-(difluoromethyl)-1-((1r,4R)-4-((4-(2-(2,4-dioxotetrahydropyrimidin-1(2H)-yl)-7-fluoro-1,3-dioxoisoindolin-5-yl)piperazin-1-yl)methyl)cyclohexyl)-1H-pyrazol-4-yl)pyrazolo[1,5-a]pyrimidine-3-carboxamide (GT-05299)

With reference to the method of Scheme T-3, the target compound (GT-05299) was obtained (yellow solid, 12 mg, yield 16.87%). ¹H NMR (400 MHz, DMSO) δ 10.88 (s, 1H), 10.18 (s, 1H), 9.51 (d, J=6.5 Hz, 1H), 8.79 (d, J=7.7 Hz, 1H), 8.41 (d, J=4.1 Hz, 1H), 8.26 (d, J=5.6 Hz, 1H), 7.45 (s, 1H), 7.26 (d, J=12.9 Hz, 1H), 7.05 (dd, J=53.6, 4.0 Hz, 1H), 4.77 (d, J=20.2 Hz, 1H), 4.27 (s, 3H), 3.82-3.69 (m, 4H), 3.57 (dd, J=30.5, 11.6 Hz, 5H), 3.45 (d, J=10.4 Hz, 2H), 3.10 (d, J=31.8 Hz, 4H), 2.84 (t, J=7.4 Hz, 2H), 2.11-2.05 (m, 2H), 2.05-1.90 (m, 5H), 1.89-1.73 (m, 2H), 1.21 (d, J=12.4 Hz, 2H). LCMS (ESI) calcd. for C₃₉H₄₂F₃N₁₂O₆⁺ [M+H]⁺: 831.33, found, 831.4.

### Example 94: Preparation of N-((1r,3r)-3-(3-chloro-4-cyanophenoxy)-2,2,4,4-tetramethylcyclobutyl)-6-(4-((4-(2-(2,4-dioxotetrahydropyrimidin-1(2H)-yl)-1,3-dioxoisoindolin-5-yl)piperazin-1-yl)methyl)piperidin-1-yl)nicotinamide (GT-05301)

With reference to the method of Scheme T-3, the target compound (GT-05301) was obtained (yellow solid, 15 mg, yield 17.29%). ¹H NMR (400 MHz, DMSO) δ 10.86 (s, 1H), 10.55-10.32 (m, 1H), 8.62 (d, J=2.3 Hz, 1H), 8.03 (d, J=7.1 Hz, 1H), 7.91 (d, J=8.8 Hz, 1H), 7.83 (d, J=8.6 Hz, 1H), 7.70 (d, J=9.0 Hz, 1H), 7.56 (d, J=2.0 Hz, 1H), 7.41 (d, J=7.1 Hz, 1H), 7.21 (d, J=2.4 Hz, 1H), 7.01 (dd, J=8.8, 2.5 Hz, 2H), 4.44 (d, J=13.7 Hz, 2H), 4.32 (s, 1H), 4.23 (d, J=13.5 Hz, 2H), 4.06 (d, J=9.0 Hz, 1H), 3.80 (t, J=7.0 Hz, 2H), 3.62 (d, J=10.7 Hz, 2H), 3.56-3.50 (m, 3H), 3.16 (d, J=7.0 Hz, 2H), 3.08 (s, 3H), 3.01 (s, 1H), 2.84 (t, J=7.0 Hz, 2H), 2.22 (s, 1H), 1.93 (d, J=11.1 Hz, 2H), 1.22 (s, 7H), 1.12 (s, 6H). LCMS (ESI) calcd. for C₄₃H₄₉ClN₉O₆⁺ [M+H]⁺: 822.35, found, 822.4.

### Example 95: Preparation of N-((1r,3S)-3-(3-chloro-4-cyanophenoxy)-2,2,4,4-tetramethylcyclobutyl)-6-(4-(((1S,4S)-5-(2-(2,4-dioxotetrahydropyrimidin-1(2H)-yl)-1,3-dioxoisoindolin-5-yl)-2,5-diazabicyclo[2.2.1]heptan-2-yl)methyl)piperidin-1-yl)nicotinamide (GT-05302)

With reference to the method of Scheme T-3, the target compound (GT-05302) was obtained (yellow solid, 29 mg, yield 32.97%). ¹H NMR (400 MHz, DMSO) δ 10.84 (d, J=3.7 Hz, 1H), 8.64-8.55 (m, 1H), 8.02 (d, J=8.2 Hz, 1H), 7.90 (d, J=8.8 Hz, 1H), 7.77 (d, J=8.4 Hz, 1H), 7.69 (d, J=8.2 Hz, 1H), 7.21 (d, J=2.4 Hz, 2H), 7.01 (dd, J=8.8, 2.4 Hz, 3H), 4.42 (d, J=11.9 Hz, 2H), 4.32 (s, 1H), 4.06 (d, J=9.2 Hz, 1H), 3.82-3.63 (m, 5H), 3.28-3.22 (m, 2H), 3.12 (s, 2H), 2.97 (s, 2H), 2.84 (t, J=6.8 Hz, 2H), 2.46-2.35 (m, 2H), 2.20 (d, J=11.2 Hz, 1H), 2.09 (d, J=13.9 Hz, 1H), 1.92 (s, 2H), 1.22 (s, 7H), 1.12 (s, 6H). LCMS (ESI) calcd. for C₄₄H₄₉ClN₉O₆⁺ [M+H]⁺: 834.35, found, 834.4.

### Example 96: Preparation of N-((1r,3R)-3-(3-chloro-4-cyanophenoxy)-2,2,4,4-tetramethylcyclobutyl)-6-(4-(((1R,4R)-5-(2-(2,4-dioxotetrahydropyrimidin-1(2H)-yl)-1,3-dioxoisoindolin-5-yl)-2,5-diazabicyclo[2.2.1]heptan-2-yl)methyl)piperidin-1-yl)nicotinamide (GT-05303)

With reference to the method of Scheme T-3, the target compound (GT-05303) was obtained (yellow solid, 16 mg, yield 18.19%). ¹H NMR (400 MHz, DMSO) δ 10.84 (d, J=3.9 Hz, 1H), 8.67-8.54 (m, 1H), 8.00 (d, J=6.5 Hz, 1H), 7.90 (d, J=8.8 Hz, 1H), 7.77 (d, J=8.4 Hz, 1H), 7.67 (d, J=10.1 Hz, 1H), 7.21 (d, J=2.4 Hz, 2H), 4.42 (d, J=13.2 Hz, 2H), 4.06 (d, J=9.1 Hz, 1H), 3.86-3.59 (m, 5H), 3.31-3.23 (m, 4H), 3.14 (d, J=22.5 Hz, 2H), 2.95 (s, 2H), 2.84 (t, J=7.0 Hz, 2H), 2.43 (d, J=10.9 Hz, 2H), 2.20 (d, J=12.2 Hz, 1H), 2.12 (s, 1H), 1.91 (s, 2H), 1.21 (d, J=2.1 Hz, 7H), 1.12 (s, 6H). LCMS (ESI) calcd, for C₄₄H₄₉ClN₉O₆⁺ [M+H]⁺: 834.35, found, 834.4.

### Example 97: Preparation of N-((1r,3r)-3-(3-chloro-4-cyanophenoxy)-2,2,4,4-tetramethylcyclobutyl)-6-(4-((4-(2-(2,4-dioxotetrahydropyrimidin-1(2H)-yl)-6-fluoro-1,3-dioxoisoindolin-5-yl)piperazin-1-yl)methyl)piperidin-1-yl)nicotinamide (GT-05304)

With reference to the method of Scheme T-3, the target compound (GT-05304) was obtained (yellow solid, 14 mg, yield 15.81%). ¹H NMR (400 MHz, DMSO) δ 10.89 (s, 1H), 8.60 (d, J=2.1 Hz, 1H), 8.10 (d, J=8.2 Hz, 1H), 7.98-7.87 (m, 2H), 7.82 (d, J=8.2 Hz, 1H), 7.67 (d, J=7.5 Hz, 1H), 7.21 (d, J=2.4 Hz, 1H), 7.10 (d, J=8.0 Hz, 1H), 7.01 (dd, J=8.8, 2.4 Hz, 1H), 4.45 (d, J=12.8 Hz, 2H), 4.33 (s, 1H), 4.07 (d, J=9.1 Hz, 1H), 3.80 (dd, J=13.0, 6.1 Hz, 4H), 3.60 (s, 5H), 3.23 (s, 2H), 3.12-3.02 (m, 4H), 2.85 (t, J=6.8 Hz, 2H), 2.24 (s, 1H), 1.96 (d, J=11.3 Hz, 2H), 1.23 (s, 7H), 1.13 (s, 6H). LCMS (ESI) calcd, for C₄₃H₄₈ClFN₉O₆⁺ [M+H]⁺: 840.34, found, 840.4.

### Example 98: Preparation of N-((1r,3r)-3-(3-chloro-4-cyanophenoxy)-2,2,4,4-tetramethylcyclobutyl)-6-(4-((8-(2-(2,4-dioxotetrahydropyrimidin-1(2H)-yl)-1,3-dioxoisoindolin-5-yl)-3,8-diazabicyclo[3.2.1]octan-3-yl)methyl)piperidin-1-yl)nicotinamide (GT-05305)

With reference to the method of Scheme T-3, the target compound (GT-05305) was obtained (yellow solid, 20 mg, yield 22.38%). ¹H NMR (400 MHz, DMSO) δ 10.86 (s, 1H), 10.19 (s, 1H), 8.57 (s, 1H), 8.07 (d, J=7.7 Hz, 1H), 7.90 (d, J=8.8 Hz, 1H), 7.82 (d, J=8.4 Hz, 2H), 7.47 (s, 1H), 7.32 (d, J=8.1 Hz, 1H), 7.21 (d, J=2.3 Hz, 1H), 7.12-6.95 (m, 2H), 4.82 (s, 2H), 4.46-4.30 (m, 3H), 4.06 (d, J=9.2 Hz, 1H), 3.79 (t, J=6.8 Hz, 2H), 3.56 (s, 2H), 3.12-2.99 (m, 4H), 2.91 (s, 2H), 2.84 (t, J=7.1 Hz, 2H), 2.41-2.32 (m, 2H), 2.18 (s, 1H), 2.11 (s, 2H), 1.86 (d, J=11.6 Hz, 2H), 1.22 (s, 8H), 1.12 (s, 6H). LCMS (ESI) calcd, for C₄₅H₅₁ClN₉O₆⁺ [M+H]⁺: 848.36, found, 848.4.

### Example 99: Preparation of N-((1r,3r)-3-(3-chloro-4-cyanophenoxy)-2,2,4,4-tetramethylcyclobutyl)-6-(4-((6-(2-(2,4-dioxotetrahydropyrimidin-1(2H)-yl)-1,3-dioxoisoindolin-5-yl)-3,6-diazabicyclo[3.1.1]heptan-3-yl)methyl)piperidin-1-yl)nicotinamide (GT-05306)

With reference to the method of Scheme T-3, the target compound (GT-05306) was obtained (yellow solid, 20 mg, yield 22.74%). ¹H NMR (400 MHz, DMSO) δ 10.86 (s, 1H), 8.56 (s, 1H), 8.05 (d, J=9.2 Hz, 1H), 7.90 (d, J=8.7 Hz, 1H), 7.84 (d, J=8.3 Hz, 1H), 7.77 (d, J=8.3 Hz, 1H), 7.28-7.16 (m, 2H), 7.02 (t, J=10.0 Hz, 3H), 4.68 (s, 2H), 4.42-4.29 (m, 3H), 4.05 (d, J=9.2 Hz, 1H), 3.78 (t, J=6.7 Hz, 4H), 3.35-3.29 (m, 2H), 2.95 (t, J=12.0 Hz, 2H), 2.84 (t, J=5.9 Hz, 5H), 2.68 (d, J=9.4 Hz, 1H), 2.09 (d, J=15.9 Hz, 1H), 1.88 (d, J=12.9 Hz, 2H), 1.19 (d, J=19.7 Hz, 8H), 1.12 (s, 6H). LCMS (ESI) calcd, for C₄₄H₄₉ClN₉O₆⁺ [M+H]⁺: 834.35, found, 834.4.

### Example 100: Preparation of N-((1r,3r)-3-(3-chloro-4-cyanophenoxy)-2,2,4,4-tetramethylcyclobutyl)-6-(4-((4-(2-(2,4-dioxotetrahydropyrimidin-1(2H)-yl)-1,3-dioxoisoindolin-4-yl)piperazin-1-yl)methyl)piperidin-1-yl)nicotinamide (GT-05345)

With reference to the method of Scheme T-3, the target compound (GT-05345) was obtained (yellow solid, 18 mg, yield 20.75%). ¹H NMR (400 MHz, DMSO) δ 10.88 (s, 1H), 10.56 (s, 1H), 8.60 (s, 1H), 8.09 (d, J=8.7 Hz, 1H), 7.91 (d, J=8.7 Hz, 1H), 7.86-7.74 (m, 2H), 7.50 (dd, J=10.6, 8.0 Hz, 2H), 7.21 (d, J=2.3 Hz, 1H), 7.09 (d, J=7.6 Hz, 1H), 7.01 (dd, J=8.8, 2.3 Hz, 1H), 4.45 (d, J=12.7 Hz, 2H), 4.33 (s, 1H), 4.07 (d, J=9.1 Hz, 1H), 3.89 (d, J=12.1 Hz, 1H), 3.83-3.77 (m, 3H), 3.69 (s, 3H), 3.57 (s, 3H), 3.26 (d, J=10.3 Hz, 3H), 3.14-3.03 (m, 4H), 2.85 (dt, J=12.5, 8.3 Hz, 2H), 2.26 (s, 1H), 1.97 (d, J=12.4 Hz, 2H), 1.25 (d, J=21.0 Hz, 8H), 1.13 (s, 6H). LCMS (ESI) calcd, for C₄₃H₄₉ClN₉O₆⁺ [M+H]⁺: 822.35, found, 822.4.

### Example 101: Preparation of N-((1r,3r)-3-(3-chloro-4-cyanophenoxy)-2,2,4,4-tetramethylcyclobutyl)-6-(4-((4-(2-(2,4-dioxotetrahydropyrimidin-1(2H)-yl)-7-fluoro-1,3-dioxoisoindolin-5-yl)piperazin-1-yl)methyl)piperidin-1-yl)nicotinamide (GT-05346)

With reference to the method of Scheme T-3, the target compound (GT-05346) was obtained (yellow solid, 36 mg, yield 40.65%). ¹H NMR (400 MHz, DMSO) δ 10.88 (s, 1H), 10.48 (s, 1H), 8.60 (s, 1H), 8.05 (d, J=8.0 Hz, 1H), 7.91 (d, J=8.8 Hz, 1H), 7.74 (d, J=8.2 Hz, 1H), 7.45 (s, 1H), 7.24 (dd, J=19.9, 7.5 Hz, 2H), 7.01 (dd, J=8.8, 2.3 Hz, 2H), 4.44 (d, J=13.0 Hz, 2H), 4.30 (d, J=17.9 Hz, 3H), 4.06 (d, J=9.1 Hz, 1H), 3.79 (dd, J=6.8, 4.8 Hz, 2H), 3.59 (t, J=12.2 Hz, 5H), 3.11 (s, 2H), 3.04 (d, J=23.7 Hz, 4H), 2.84 (t, J=7.2 Hz, 2H), 2.23 (s, 1H), 1.94 (d, J=10.7 Hz, 2H), 1.22 (s, 7H), 1.13 (s, 6H). LCMS (ESI) calcd, for C₄₃H₄₈ClFN₉O₆⁺ [M+H]⁺: 840.34, found, 840.3.

### Example 102: Preparation of N-((1r,3r)-3-(3-chloro-4-cyanophenoxy)-2,2,4,4-tetramethylcyclobutyl)-6-(4-((4-(1-(2-(2,4-dioxotetrahydropyrimidin-1(2H)-yl)-1,3-dioxoisoindolin-5-yl)-3,3-difluoropiperidin-4-yl)piperazin-1-yl)methyl)piperidin-1-yl)nicotinamide (GT-05347)

With reference to the method of Scheme T-3, the target compound (GT-05347) was obtained (yellow solid, 32 mg, yield 31.23%). ¹H NMR (400 MHz, DMSO) δ 10.85 (s, 1H), 9.95 (s, 1H), 8.58 (s, 1H), 8.12 (d, J=8.4 Hz, 1H), 7.91 (d, J=8.8 Hz, 1H), 7.84 (s, 1H), 7.75 (d, J=8.6 Hz, 1H), 7.52 (s, 1H), 7.41 (d, J=8.7 Hz, 1H), 7.21 (d, J=2.3 Hz, 1H), 7.13 (s, 1H), 7.01 (dd, J=8.8, 2.3 Hz, 1H), 4.52-4.37 (m, 3H), 4.33 (s, 1H), 4.26 (d, J=12.7 Hz, 1H), 4.07 (d, J=9.1 Hz, 1H), 3.79 (t, J=6.6 Hz, 2H), 3.35 (d, J=14.5 Hz, 4H), 3.07 (dd, J=32.9, 19.3 Hz, 10H), 2.83 (t, J=7.0 Hz, 2H), 2.17 (s, 1H), 1.88 (s, 4H), 1.23 (s, 8H), 1.12 (s, 6H). LCMS (ESI) calcd, for C₄₈H₅₆ClF₂N₁₀O₆⁺ [M+H]⁺: 941.40, found, 941.4.

### Example 103: Preparation of 6-(5-cyano-1H-pyrrolo[2,3-b]pyridin-1-yl)-N-(1-((1-(2-(2,4-dioxotetrahydropyrimidin-1(2H)-yl)-1,3-dioxoisoindolin-5-yl)piperidin-4-yl)methyl)piperidin-4-yl)-4-(isopropylamino) nicotinamide (GT-05813)

With reference to the method of Scheme T-3, the target compound (GT-05813) was obtained (yellow solid, 26 mg, yield 34.61%). ¹H NMR (400 MHz, DMSO-d₆) δ 10.84 (s, 1H), 10.13 (s, 1H), 8.85-8.78 (m, 2H), 8.69 (d, J=2.1 Hz, 2H), 8.51 (t, J=3.2 Hz, 1H), 8.03 (d, J=5.4 Hz, 1H), 7.72 (d, J=8.6 Hz, 1H), 7.41 (s, 1H), 7.31 (d, J=8.6 Hz, 1H), 6.93 (dd, J=3.8, 2.1 Hz, 1H), 4.07 (dd, J=40.7, 9.6 Hz, 3H), 3.79 (dd, J=15.8, 9.0 Hz, 3H), 3.36 (s, 2H), 3.01 (dd, J=24.3, 12.2 Hz, 6H), 2.83 (t, J=7.1 Hz, 2H), 2.28-2.09 (m, 2H), 2.05 (d, J=11.3 Hz, 3H), 1.93 (d, J=10.8 Hz, 2H), 1.29 (d, J=6.3 Hz, 8H). LCMS (ESI) calcd, for C₄₀H₄₄N₁₁O₅⁺ [M+H]⁺: 758.35, found, 758.4.

### Example 104: Preparation of 6-(5-cyano-1H-pyrrolo[2,3-b]pyridin-1-yl)-N-(1-((1-(2-(2,4-dioxotetrahydropyrimidin-1(2H)-yl)-1,3-dioxoisoindolin-4-yl)piperidin-4-yl)methyl)piperidin-4-yl)-4-(isopropylamino) nicotinamide (GT-05814)

With reference to the method of Scheme T-3, the target compound (GT-05814) was obtained (yellow solid, 40 mg, yield 53.24%). ¹H NMR (400 MHz, DMSO-d₆) δ 10.86 (s, 1H), 10.34 (s, 1H), 8.95-8.83 (m, 2H), 8.82 (d, J=1.9 Hz, 1H), 8.74-8.68 (m, 2H), 8.51 (t, J=3.8 Hz, 1H), 7.99 (d, J=4.0 Hz, 1H), 7.80-7.69 (m, 1H), 7.39 (t, J=8.0 Hz, 2H), 6.94 (dd, J=3.9, 1.8 Hz, 1H), 4.24-3.97 (m, 1H), 3.88-3.75 (m, 5H), 3.59 (d, J=12.0 Hz, 3H), 3.39 (s, 1H), 3.09-2.80 (m, 7H), 2.19-1.98 (m, 6H), 1.57-1.38 (m, 2H), 1.29 (d, J=6.3 Hz, 6H). LCMS (ESI) calcd, for C₄₀H₄₄N₁₁O₅⁺ [M+H]⁺: 758.35, found, 758.4.

### Example 105: Preparation of N-(2-(1-((1-(2-(2,4-dioxotetrahydropyrimidin-1(2H)-yl)-1,3-dioxoisoindolin-4-yl)piperidin-4-yl)methyl)piperidin-4-yl)-6-methoxy-2H-indazol-5-yl)-3-(trifluoromethyl)benzamide (GT-05815)

With reference to the method of Scheme T-3, the target compound (GT-05815) was obtained (yellow solid, 32 mg, yield 43.31%). ¹H NMR (400 MHz, DMSO-d₆) δ 10.86 (s, 1H), 10.01 (d, J=77.9 Hz, 1H), 9.81 (d, J=6.9 Hz, 1H), 8.51-8.22 (m, 3H), 8.06 (s, 1H), 7.98 (d, J=7.4 Hz, 1H), 7.82-7.72 (m, 2H), 7.42 (t, J=8.3 Hz, 2H), 7.10 (s, 1H), 4.77 (s, 1H), 3.88 (s, 3H), 3.84-3.70 (m, 5H), 3.42 (s, 1H), 3.26-3.10 (m, 4H), 2.90 (ddd, J=21.4, 20.9, 9.3 Hz, 4H), 2.58 (d, J=12.3 Hz, 2H), 2.36 (d, J=13.8 Hz, 2H), 1.99 (d, J=12.4 Hz, 3H), 1.46 (d, J=8.6 Hz, 2H). LCMS (ESI) calcd, for C₃₉H₄₀F₃N₈O₆⁺ [M+H]⁺: 773.30, found, 773.3.

### Example 106: Preparation of N-(2-(1-((1-(2-(2,4-dioxotetrahydropyrimidin-1(2H)-yl)-1,3-dioxoisoindolin-4-yl)piperidin-4-yl)methyl)piperidin-4-yl)-6-methoxy-2H-indazol-5-yl)-6-(trifluoromethyl) picolinamide (GT-05817)

With reference to the method of Scheme T-3, the target compound (GT-05817) was obtained (yellow solid, 40 mg, yield 54.20%). ¹H NMR (400 MHz, DMSO-d₆) δ 10.87 (s, 1H), 10.53 (s, 1H), 9.95 (d, J=78.8 Hz, 1H), 8.73 (d, J=2.4 Hz, 1H), 8.54-8.32 (m, 3H), 8.23 (d, J=7.6 Hz, 1H), 7.83-7.65 (m, 1H), 7.42 (t, J=8.2 Hz, 2H), 7.19 (d, J=3.9 Hz, 1H), 4.79 (t, J=27.3 Hz, 1H), 4.00 (s, 3H), 3.82-3.69 (m, 5H), 3.49 (d, J=4.4 Hz, 1H), 3.20 (dd, J=32.6, 21.2 Hz, 5H), 3.04-2.78 (m, 4H), 2.56 (d, J=13.0 Hz, 2H), 2.35 (d, J=13.6 Hz, 2H), 2.12 (s, 1H), 1.99 (d, J=11.3 Hz, 2H), 1.58-1.36 (m, 2H). LCMS (ESI) calcd, for C₃₈H₃₉F₃N₉O₆⁺ [M+H]⁺: 774.30, found, 774.3.

### Example 107: Preparation of N-(2-(1-((2-(2,4-dioxotetrahydropyrimidin-1(2H)-yl)-1-oxoisoindolin-5-yl)methyl)piperidin-4-yl)-6-(2-hydroxypropan-2-yl)-2H-indazol-5-yl)-6-(trifluoromethyl) picolinamide (GT-05746)

With reference to the method of Step 2 in Scheme T-1, the target compound (GT-05746) was obtained (yellow solid, 20 mg, yield 31.75%). ¹HNMR (400 MHz, DMSO-d₆) δ 10.86 (dd, J=20.0, 13.4 Hz, 1H), 9.39 (s, 1H), 9.17-8.98 (m, 1H), 7.80 (dd, J=12.9, 5.3 Hz, 1H), 7.65 (t, J=8.2 Hz, 1H), 7.56-7.27 (m, 2H), 7.19-7.07 (m, 1H), 6.91-6.73 (m, 2H), 6.45-6.15 (m, 1H), 4.80-4.37 (m, 4H), 4.31-4.20 (m, 2H), 3.82 (s, 6H), 3.20 (s, 6H), 2.90 (ddd, J=30.5, 15.6, 7.4 Hz, 2H), 2.63-2.55 (m, 2H), 2.16 (s, 1H). LCMS (ESI) calcd. for C₃₅H₃₆F₃N₈O₅⁺ [M+H]⁺: 705.28, found, 705.3.

### Example 108: Preparation of N-(2-(1-((2-(2,4-dioxotetrahydropyrimidin-1(2H)-yl)-1,3-dioxoisoindolin-5-yl)methyl)piperidin-4-yl)-6-(2-hydroxypropan-2-yl)-2H-indazol-5-yl)-6-(trifluoromethyl) picolinamide (GT-05752)

With reference to the method of Step 2 in Scheme T-1, the target compound was obtained (GT-05752) (yellow solid, 23 mg, yield 35.80%). ¹H NMR (400 MHz, DMSO) δ 12.39 (s, 1H), 11.32 (s, 1H), 10.93 (d, J=8.7 Hz, 1H), 8.74 (s, 1H), 8.46 (s, 1H), 8.42-8.34 (m, 3H), 8.27-8.08 (m, 3H), 7.60 (s, 1H), 4.84 (dd, J=39.7, 28.1 Hz, 1H), 4.61 (s, 2H), 3.83 (dt, J=6.9, 5.4 Hz, 2H), 3.58 (d, J=12.2 Hz, 2H), 3.25 (s, 3H), 2.87 (t, J=6.8 Hz, 2H), 2.77-2.59 (m, 1H), 2.35 (d, J=13.0 Hz, 2H), 1.63 (d, J=9.2 Hz, 6H). LCMS (ESI) calcd. for C₃₅H₃₄F₃N₈O₆⁺ [M+H]⁺: 719.26, found, 719.3.

### Example 109: Preparation of N-(2-(1-((2-(2,4-dioxotetrahydropyrimidin-1(2H)-yl)-1-oxoisoindolin-5-yl)methyl)piperidin-4-yl)-5-(2-hydroxypropan-2-yl)benzo[d]thiazol-6-yl)-6-(trifluoromethyl) picolinamide (GT-05747)

With reference to the method of Step 2 in Scheme T-1, the target compound (GT-05747) was obtained (yellow solid, 23 mg, yield 37.01%). ¹H NMR (400 MHz, DMSO) δ 12.62 (s, 1H), 10.86 (s, 1H), 10.70 (d, J=3.6 Hz, 1H), 8.76 (d, J=12.6 Hz, 1H), 8.43 (dd, J=28.2, 7.7 Hz, 2H), 8.19 (d, J=7.0 Hz, 1H), 7.90 (d, J=8.1 Hz, 2H), 7.81 (d, J=8.5 Hz, 1H), 7.71 (d, J=14.5 Hz, 1H), 6.09 (s, 1H), 4.82 (t, J=14.2 Hz, 1H), 4.60 (d, J=16.7 Hz, 1H), 4.48 (s, 2H), 3.80 (t, J=6.7 Hz, 2H), 3.51 (s, 2H), 3.14 (d, J=4.3 Hz, 2H), 2.79 (s, 2H), 2.35 (d, J=12.9 Hz, 2H), 2.25-2.12 (m, 2H), 2.09-1.93 (m, 1H), 1.62 (d, J=11.6 Hz, 6H). LCMS (ESI) calcd. for C₃₅H₃₅F₃N₇O₅S⁺ [M+H]⁺: 722.24, found, 722.3.

### Example 110: Preparation of N-(2-(1-((2-(2,4-dioxotetrahydropyrimidin-1(2H)-yl)-1,3-dioxoisoindolin-5-yl)methyl)piperidin-4-yl)-5-(2-hydroxypropan-2-yl)benzo[d]thiazol-6-yl)-6-(trifluoromethyl) picolinamide (GT-05762)

With reference to the method of Step 2 in Scheme T-1, the target compound (GT-05762) was obtained (yellow solid, 28 mg, yield 44.19%). ¹H NMR (400 MHz, DMSO-d₆) δ 12.58 (d, J=8.4 Hz, 1H), 11.27 (s, 1H), 9.12 (d, J=10.3 Hz, 1H), 8.52-8.44 (m, 1H), 8.38 (dd, J=14.1, 6.1 Hz, 2H), 8.21 (dd, J=11.5, 7.7 Hz, 2H), 8.16-8.01 (m, 2H), 7.94 (d, J=13.6 Hz, 1H), 6.12 (s, 1H), 4.53 (d, J=40.4 Hz, 2H), 3.83 (q, J=6.9 Hz, 3H), 3.53 (d, J=11.2 Hz, 2H), 3.16 (d, J=10.6 Hz, 2H), 2.86 (q, J=6.7 Hz, 3H), 2.28 (dd, J=35.1, 11.7 Hz, 3H), 1.65 (d, J=10.2 Hz, 6H). LCMS (ESI) calcd, for C₃₅H₃₃F₃N₇O₆S⁺ [M+H]⁺: 736.22, found, 736.3.

### Example 111: Preparation of N-(2-(1-((1-(2-(2,4-dioxotetrahydropyrimidin-1(2H)-yl)-1,3-dioxoisoindolin-5-yl)piperidin-4-yl)methyl)piperidin-4-yl)-5-(2-hydroxypropan-2-yl)benzo[d]thiazol-6-yl)-6-(trifluoromethyl) picolinamide (GT-05819)

With reference to the method of Scheme T-3, the target compound (GT-05819) was obtained (yellow solid, 38 mg, yield 53.89%). ¹H NMR (400 MHz, DMSO-d₆) δ 12.58 (s, 1H), 10.85 (s, 1H), 9.79 (s, 1H), 9.11 (d, J=4.3 Hz, 1H), 8.47 (d, J=7.8 Hz, 1H), 8.39 (t, J=7.8 Hz, 1H), 8.20 (d, J=8.4 Hz, 1H), 7.93 (d, J=4.2 Hz, 1H), 7.73 (dd, J=8.6, 5.3 Hz, 1H), 7.42 (t, J=3.7 Hz, 1H), 7.33 (dd, J=11.1, 4.4 Hz, 1H), 6.12 (s, 1H), 4.14 (d, J=13.1 Hz, 2H), 3.79 (t, J=6.9 Hz, 2H), 3.67 (d, J=10.9 Hz, 2H), 3.46 (s, 3H), 3.09 (dd, J=23.4, 12.2 Hz, 5H), 2.83 (t, J=7.2 Hz, 2H), 2.35-2.18 (m, 4H), 1.93 (d, J=11.4 Hz, 2H), 1.64 (s, 6H), 1.30 (d, J=12.2 Hz, 2H). LCMS (ESI) calcd, for C₄₀H₄₂F₃N₈O₆S⁺ [M+H]⁺: 819.29, found, 819.3.

### Example 112: Preparation of N-(2-(1-((1-(2-(2,4-dioxotetrahydropyrimidin-1(2H)-yl)-1,3-dioxoisoindolin-4-yl)piperidin-4-yl)methyl)piperidin-4-yl)-5-(2-hydroxypropan-2-yl)benzo[d]thiazol-6-yl)-6-(trifluoromethyl) picolinamide (GT-05818)

With reference to the method of Scheme T-3, the target compound (GT-05818) was obtained (yellow solid, 38 mg, yield 53.89%). ¹H NMR (400 MHz, DMSO-d₆) δ 12.58 (s, 1H), 10.86 (d, J=3.5 Hz, 1H), 10.12 (s, 1H), 9.11 (d, J=6.1 Hz, 1H), 8.48 (d, J=7.7 Hz, 1H), 8.40 (t, J=7.9 Hz, 1H), 8.20 (d, J=7.8 Hz, 1H), 7.99-7.91 (m, 1H), 7.75 (ddd, J=11.6, 7.2, 4.5 Hz, 1H), 7.45-7.36 (m, 2H), 3.86-3.67 (m, 6H), 3.52 (s, 2H), 3.12 (d, J=21.2 Hz, 4H), 3.01-2.80 (m, 4H), 2.33 (s, 3H), 2.08 (dd, J=59.9, 16.3 Hz, 3H), 1.65 (s, 6H), 1.49 (dd, J=29.0, 10.6 Hz, 2H). LCMS (ESI) calcd, for C₄₀H₄₂F₃N₈O₆S⁺ [M+H]⁺: 819.29, found, 819.3.

### Example 113: Preparation of N-(2-(1-((2-(2,4-dioxotetrahydropyrimidin-1(2H)-yl)-1-oxoisoindolin-5-yl)methyl)piperidin-4-yl)-5-(2-hydroxypropan-2-yl)benzo[d]oxazol-6-yl)-6-(trifluoromethyl) picolinamide (GT-05748)

With reference to the method of Step 2 in Scheme T-1, the target compound (GT-05748) was obtained (yellow solid, 18 mg, yield 28.59%). ¹H NMR (400 MHz, DMSO) § 12.62 (s, 1H), 10.86 (s, 1H), 10.70 (d, J=3.6 Hz, 1H), 8.76 (d, J=12.6 Hz, 1H), 8.43 (dd, J=28.2, 7.7 Hz, 2H), 8.19 (d, J=7.0 Hz, 1H), 7.90 (d, J=8.1 Hz, 2H), 7.81 (d, J=8.5 Hz, 1H), 7.71 (d, J=14.5 Hz, 1H), 6.09 (s, 1H), 4.82 (t, J=14.2 Hz, 1H), 4.60 (d, J=16.7 Hz, 1H), 4.48 (s, 2H), 3.80 (t, J=6.7 Hz, 2H), 3.51 (s, 2H), 3.14 (d, J=4.3 Hz, 2H), 2.79 (s, 2H), 2.35 (d, J=12.9 Hz, 2H), 2.25-2.12 (m, 2H), 2.09-1.93 (m, 1H), 1.62 (d, J=11.6 Hz, 6H). LCMS (ESI) calcd. for C₃₅H₃₅F₃N₇O₆⁺ [M+H]⁺: 706.26, found, 706.3.

### Example 114: Preparation of N-(2-(1-((2-(2,4-dioxotetrahydropyrimidin-1(2H)-yl)-1,3-dioxoisoindolin-5-yl)methyl)piperidin-4-yl)-5-(2-hydroxypropan-2-yl)benzo[d]oxazol-6-yl)-6-(trifluoromethyl) picolinamide (GT-05770)

With reference to the method of Step 2 in Scheme T-1, the target compound (GT-05770) was obtained (yellow solid, 16 mg, yield 24.93%). ¹HNMR (400 MHz, DMSO-d₆) δ 12.63 (s, 1H), 11.14 (s, 1H), 10.94 (s, 1H), 8.75 (s, 1H), 8.47-8.32 (m, 3H), 8.22-8.07 (m, 4H), 7.70 (s, 1H), 6.08 (s, 1H), 4.58 (s, 2H), 3.83 (d, J=6.7 Hz, 2H), 3.57 (dd, J=36.9, 11.7 Hz, 2H), 3.07 (d, J=56.9 Hz, 3H), 2.87 (t, J=6.5 Hz, 2H), 2.35 (d, J=13.6 Hz, 2H), 2.26-2.03 (m, 2H), 1.62 (d, J=11.6 Hz, 6H). LCMS (ESI) calcd, for C₃₅H₃₃F₃N₇O₇⁺ [M+H]⁺: 720.24, found, 720.3.

### Example 115: Preparation of N-(2-(1-((1′-(2-(2,4-dioxotetrahydropyrimidin-1(2H)-yl)-1,3-dioxoisoindolin-5-yl)-[1,4′-bipiperidin]-4-yl)methyl)piperidin-4-yl)-5-(2-hydroxypropan-2-yl)benzo[d]oxazol-6-yl)-6-(trifluoromethyl) picolinamide (GT-05759)

With reference to the method of Scheme T-3, the target compound (GT-05759) was obtained (yellow solid, 11 mg, yield 11.14%). ¹H NMR (400 MHz, DMSO-d₆) δ 12.62 (s, 1H), 10.84 (s, 1H), 10.67-10.25 (m, 2H), 8.75 (d, J=4.3 Hz, 1H), 8.46 (d, J=7.8 Hz, 1H), 8.38 (t, J=7.8 Hz, 1H), 8.19 (d, J=7.8 Hz, 1H), 7.46 (s, 1H), 7.35 (d, J=8.7 Hz, 1H), 6.08 (s, 1H), 4.27 (d, J=12.3 Hz, 2H), 3.78 (t, J=6.8 Hz, 2H), 3.71-3.44 (m, 8H), 3.12-2.92 (m, 7H), 2.83 (t, J=6.9 Hz, 2H), 2.17 (dd, J=64.1, 36.5 Hz, 9H), 1.71 (dd, J=22.8, 11.3 Hz, 3H), 1.61 (s, 6H). LCMS (ESI) calcd, for C₄₅H₅₁F₃N₉O₇⁺ [M+H]⁺: 886.39, found, 886.4.

### Example 116: Preparation of N-(3-(difluoromethyl)-1-(1-((2-(2,4-dioxotetrahydropyrimidin-1(2H)-yl)-1-oxoisoindolin-5-yl)methyl)piperidin-4-yl)-1H-pyrazol-4-yl)-5-morpholinopyrazolo[1,5-alpyrimidine-3-carboxamide (GT-05749)

With reference to the method of Step 2 in Scheme T-1, the target compound (GT-05749) was obtained (yellow solid, 20 mg, yield 31.72%). ¹H NMR (400 MHz, DMSO-d₆) δ 10.86 (d, J=44.0 Hz, 1H), 10.69 (s, 1H), 9.42 (s, 1H), 8.82 (d, J=7.9 Hz, 1H), 8.42 (s, 1H), 8.29 (s, 1H), 7.94-7.87 (m, 2H), 7.80 (d, J=8.0 Hz, 1H), 7.13 (t, J=53.6 Hz, 1H), 6.91 (d, J=7.9 Hz, 1H), 4.83 (d, J=16.9 Hz, 1H), 4.59 (d, J=19.4 Hz, 2H), 4.49 (s, 1H), 3.76 (dd, J=26.0, 5.1 Hz, 10H), 3.53 (d, J=11.2 Hz, 3H), 3.13 (dd, J=7.4, 4.2 Hz, 2H), 2.84 (dd, J=22.3, 15.0 Hz, 2H), 2.29 (dd, J=33.1, 11.2 Hz, 4H). LCMS (ESI) calcd, for C₃₃H₃₆F₂N₁₁O₅⁺ [M+H]⁺: 704.29, found, 704.3.

### Example 117: Preparation of N-(3-(difluoromethyl)-1-(1-((1′-(2-(2,4-dioxotetrahydropyrimidin-1(2H)-yl)-1,3-dioxoisoindolin-5-yl)-[1,4′-bipiperidin]-4-yl)methyl)piperidin-4-yl)-1H-pyrazol-4-yl)-5-morpholinopyrazolo[1,5-a]pyrimidine-3-carboxamide (GT-05760)

With reference to the method of Scheme T-3, the target compound (GT-05760) was obtained (yellow solid, 16.16 mg, yield 16.16%). ¹H NMR (400 MHz, DMSO) δ 10.85 (s, 1H), 10.66-9.97 (m, 2H), 9.44 (s, 1H), 8.83 (d, J=7.9 Hz, 1H), 8.48 (t, J=22.6 Hz, 1H), 8.30 (s, 1H), 7.79-7.44 (m, 1H), 7.14 (t, J=53.6 Hz, 1H), 6.92 (d, J=8.0 Hz, 1H), 4.58 (dd, J=22.7, 16.9 Hz, 1H), 4.28 (d, J=12.3 Hz, 1H), 3.85-3.63 (m, 12H), 3.56-3.42 (m, 12H), 3.22-3.16 (m, 3H), 2.99 (dd, J=26.3, 12.7 Hz, 3H), 2.84 (t, J=7.1 Hz, 1H), 2.26 (ddd, J=55.2, 29.8, 23.3 Hz, 6H), 1.72 (dd, J=24.8, 11.6 Hz, 2H). LCMS (ESI) calcd, for C₄₃H₅₂F₂N₁₃O₆⁺ [M+H]⁺: 884.41, found, 884.5.

### Example 118: Preparation of 5-((1R,4R)-2-oxa-5-azabicyclo[2.2.1]heptan-5-yl)-N-(3-(difluoromethyl)-1-(1-((1′-(2-(2,4-dioxotetrahydropyrimidin-1(2H)-yl)-1,3-dioxoisoindolin-5-yl)-[1,4′-bipiperidin]-4-yl)methyl)piperidin-4-yl)-1H-pyrazol-4-yl)pyrazolo[1,5-a]pyrimidine-3-carboxamide (GT-05816)

With reference to the method of Scheme T-3, the target compound (GT-05816) was obtained (yellow solid, 9 mg, yield 11.51%). ¹H NMR (400 MHz, DMSO-d₆) δ 10.86 (s, 1H), 9.55 (d, J=7.9 Hz, 2H), 8.74 (d, J=7.8 Hz, 1H), 8.45 (s, 1H), 8.31-8.25 (m, 1H), 7.76 (d, J=8.6 Hz, 1H), 7.45 (s, 1H), 7.36 (d, J=8.8 Hz, 1H), 7.12 (s, 1H), 6.88-6.42 (m, 1H), 5.15 (d, J=89.5 Hz, 1H), 4.76 (d, J=21.3 Hz, 1H), 4.26 (d, J=13.5 Hz, 2H), 3.87-3.72 (m, 12H), 3.38-3.32 (m, 7H), 3.01 (d, J=12.4 Hz, 6H), 2.84 (t, J=6.9 Hz, 2H), 2.28 (s, 2H), 2.13 (s, 2H), 2.07-1.96 (m, 3H), 1.71 (s, 2H), 1.53 (s, 1H). LCMS (ESI) calcd, for C₄₄H₅₂F₂N₁₃O₆⁺ [M+H]⁺: 896.41, found, 896.4.

### Example 119: Preparation of (R)—N-(2-(4-((2-(2,4-dioxotetrahydropyrimidin-1(2H)-yl)-1-oxoisoindolin-5-yl)methyl)piperazin-1-yl)-5-(3-hydroxypyrrolidin-1-yl) oxazolo[4,5-b]pyridin-6-yl)-2-(2-methylpyridin-4-yl) oxazole-4-carboxamide (GT-05750)

With reference to the method of Step 2 in Scheme T-1, the target compound (GT-05750) was obtained (yellow solid, 28 mg, yield 45.92%). ¹H NMR (400 MHz, DMSO) δ 11.97 (s, 1H), 10.70 (s, 1H), 10.08 (s, 1H), 9.18 (s, 1H), 8.93 (d, J=6.1 Hz, 1H), 8.39 (s, 1H), 8.28 (d, J=6.0 Hz, 1H), 7.94 (s, 1H), 7.85 (dd, J=18.0, 7.9 Hz, 2H), 7.72 (s, 1H), 4.85-4.51 (m, 4H), 4.29 (s, 4H), 3.65-3.56 (m, 4H), 3.48-3.27 (m, 6H), 2.96-2.67 (m, 6H), 1.91 (d, J=8.5 Hz, 1H), 1.79 (d, J=4.3 Hz, 1H).LCMS (ESI) calcd, for C₃₇H₃₈N₁₁O₇⁺ [M+H]⁺: 748.30, found, 748.3.

### Example 120: Preparation of (R)—N-(2-(4-((2-(2,4-dioxotetrahydropyrimidin-1(2H)-yl)-1,3-dioxoisoindolin-5-yl)methyl)piperazin-1-yl)-5-(3-hydroxypyrrolidin-1-yl) oxazolo[4,5-b]pyridin-6-yl)-2-(2-methylpyridin-4-yl) oxazole-4-carboxamide (GT-05803)

With reference to the method of Step 2 in Scheme T-1, the target compound (GT-05803) was obtained (yellow solid, 19 mg, yield 30.59%). ¹H NMR (400 MHz, DMSO-d₆) δ 12.00-11.31 (m, 1H), 10.48 (s, 1H), 10.03 (s, 1H), 9.16 (d, J=2.0 Hz, 1H), 8.90 (d, J=6.0 Hz, 1H), 8.34 (d, J=5.1 Hz, 1H), 8.22 (d, J=5.2 Hz, 1H), 7.93 (s, 1H), 7.71 (s, 1H), 7.49 (s, 1H), 4.57 (d, J=42.2 Hz, 3H), 4.28 (s, 3H), 3.62-3.59 (m, 2H), 3.33 (dd, J=50.5, 29.5 Hz, 9H), 2.86 (s, 1H), 2.81 (s, 3H), 2.77 (d, J=6.3 Hz, 1H), 1.98-1.85 (m, 1H), 1.78 (s, 1H). LCMS (ESI) calcd, for C₃₇H₃₆N₁₁O₈⁺ [M+H]⁺: 762.27, found, 762.3.

### Example 121: Preparation of (R)—N-(2-(4-((1-(2-(2,4-dioxotetrahydropyrimidin-1(2H)-yl)-1,3-dioxoisoindolin-5-yl)piperidin-4-yl)methyl)piperazin-1-yl)-5-(3-hydroxypyrrolidin-1-yl) oxazolo[4,5-b]pyridin-6-yl)-2-(2-methylpyridin-4-yl) oxazole-4-carboxamide (GT-05805)

With reference to the method of Scheme T-3, the target compound (GT-05805) was obtained (yellow solid, 16 mg, yield 23.22%). ¹H NMR (400 MHz, DMSO-d₆) δ 11.08 (s, 1H), 10.49 (dd, J=120.0, 84.2 Hz, 1H), 10.06 (d, J=4.7 Hz, 1H), 9.68 (d, J=100.1 Hz, 1H), 9.16 (d, J=3.9 Hz, 1H), 8.91 (d, J=6.0 Hz, 1H), 8.36 (s, 1H), 8.25 (d, J=5.9 Hz, 1H), 7.76-7.68 (m, 1H), 7.44-7.31 (m, 1H), 4.41-4.03 (m, 8H), 3.47-3.17 (m, 8H), 3.12-2.91 (m, 5H), 2.86-2.80 (m, 4H), 2.73 (t, J=6.5 Hz, 1H), 2.19 (s, 1H), 2.04-1.75 (m, 4H), 1.42-1.20 (m, 2H). LCMS (ESI) calcd, for C₄₂H₄₅N₁₂O₈⁺ [M+H]⁺: 845.35, found, 845.4.

### Example 122: Preparation of (R)—N-(2-(4-((1-(2-(2,4-dioxotetrahydropyrimidin-1(2H)-yl)-1,3-dioxoisoindolin-4-yl)piperidin-4-yl)methyl)piperazin-1-yl)-5-(3-hydroxypyrrolidin-1-yl) oxazolo[4,5-b]pyridin-6-yl)-2-(2-methylpyridin-4-yl) oxazole-4-carboxamide (GT-05806)

With reference to the method of Scheme T-3, the target compound (GT-05806) was obtained (yellow solid, 5 mg, yield 7.22%). ¹H NMR (400 MHz, DMSO-d₆) δ 11.02 (s, 1H), 10.86 (s, 1H), 10.02 (d, J=9.0 Hz, 1H), 9.14 (d, J=2.9 Hz, 1H), 8.89 (d, J=5.9 Hz, 1H), 8.30 (s, 1H), 8.19 (d, J=5.8 Hz, 1H), 7.75-7.69 (m, 1H), 7.54-7.37 (m, 2H), 4.33-4.18 (m, 3H), 3.85 (s, 6H), 3.47 (dd, J=7.8, 3.9 Hz, 6H), 3.27 (d, J=9.1 Hz, 3H), 3.14 (s, 2H), 2.96 (dd, J=20.6, 9.9 Hz, 2H), 2.83 (dd, J=10.2, 7.0 Hz, 2H), 2.79 (s, 3H), 2.03 (s, 3H), 1.95-1.85 (m, 1H), 1.79 (s, 1H), 1.46 (s, 2H). LCMS (ESI) calcd, for C₄₂H₄₅N₁₂O₈⁺ [M+H]⁺: 845.35, found, 845.4.

### Example 123: Preparation of N-(1-(1-((1-(2-(2,4-dioxotetrahydropyrimidin-1(2H)-yl)-1,3-dioxoisoin dolin-5-yl)piperidin-4-yl)methyl)piperidin-4-yl)-6-methoxy-1H-indazol-7-yl)-3-(trifluoromethyl)benzamide (GT-06168)

With reference to the method of Scheme T-3, the target compound (GT-06168) was obtained (yellow solid, 11 mg, yield 9.93%). ¹H NMR (400 MHz, DMSO-d₆) δ 10.84 (s, 1H), 10.32 (d, J=17.7 Hz, 1H), 9.97 (d, J=36.0 Hz, 1H), 8.45-8.35 (m, 2H), 8.17-8.10 (m, 1H), 8.05 (d, J=7.7 Hz, 1H), 7.86 (dd, J=9.7, 5.9 Hz, 1H), 7.82-7.76 (m, 1H), 7.71 (dd, J=8.6, 2.9 Hz, 1H), 7.40 (d, J=1.9 Hz, 1H), 7.30 (dd, J=8.8, 2.1 Hz, 1H), 7.15 (t, J=8.6 Hz, 1H), 4.89 (s, 1H), 4.09 (d, J=12.5 Hz, 2H), 3.85 (d, J=2.8 Hz, 3H), 3.78 (t, J=6.8 Hz, 2H), 3.71-3.50 (m, 3H), 2.99 (t, J=11.9 Hz, 3H), 2.85 (dd, J=23.9, 16.9 Hz, 4H), 2.70-2.54 (m, 2H), 2.14 (d, J=47.3 Hz, 3H), 1.85 (d, J=12.1 Hz, 2H), 1.24 (s, 2H). LCMS (ESI) calcd, for C₃₉H₄₀F₃N₈O₆⁺ [M+H]⁺: 773.30, found, 773.3.

### Example 124: Preparation of 5-((1R,4R)-2-oxa-5-azabicyclo[2.2.1]heptan-5-yl)-N-(3-(difluoromethyl)-1-((1r,4R)-4-((3-(2-(2,4-dioxotetrahydropyrimidin-1(2H)-yl)-1,3-dioxoisoindolin-5-yl)-3,6-diazabicyclo[3.1.1]heptan-6-yl)methyl)cyclohexyl)-1H-pyrazol-4-yl)pyrazolo[1,5-a]pyrimidine-3-carboxamide (GT-05822)

With reference to the method of Scheme T-3, the target compound (GT-05822) was obtained (yellow solid, 13 mg, yield 19.13%). ¹H NMR (400 MHz, DMSO-d₆) δ 10.86 (s, 1H), 9.51 (s, 1H), 8.78 (d, J=7.6 Hz, 1H), 8.41 (dd, J=8.0, 4.1 Hz, 1H), 8.25 (d, J=4.5 Hz, 1H), 7.89-7.82 (m, 1H), 7.29-7.00 (m, 2H), 6.66 (dd, J=164.2, 7.8 Hz, 1H), 5.18 (d, J=80.1 Hz, 1H), 4.85-4.63 (m, 2H), 4.52-3.96 (m, 4H), 3.86-3.70 (m, 4H), 3.67-3.55 (m, 2H), 3.45 (d, J=11.7 Hz, 3H), 3.30-3.22 (m, 2H), 2.93 (d, J=73.8 Hz, 4H), 2.16-1.87 (m, 7H), 1.78 (d, J=12.4 Hz, 3H). LCMS (ESI) calcd, for C₄₀H₄₃F₂N₁₂O₆⁺ [M+H]⁺: 825.34, found, 825.4.

### Example 125: Preparation of 5-((1R,4R)-2-oxa-5-azabicyclo[2.2.1]heptan-5-yl)-N-(3-(difluoromethyl)-1-((1r,4R)-4-((3-(2-(2,4-dioxotetrahydropyrimidin-1(2H)-yl)-1,3-dioxoisoindolin-5-yl)-3,8-diazabicyclo[3.2.1]octan-8-yl)methyl)cyclohexyl)-1H-pyrazol-4-yl)pyrazolo[1,5-a]pyrimidine-3-carboxamide (GT-05825)

With reference to the method of Scheme T-3, the target compound (GT-05825) was obtained (yellow solid, 32 mg, yield 46.30%). ¹H NMR (400 MHz, DMSO-d₆) δ 10.86 (s, 1H), 10.03 (s, 1H), 9.52 (d, J=6.5 Hz, 1H), 8.79 (d, J=7.8 Hz, 1H), 8.44 (t, J=12.8 Hz, 1H), 8.26 (d, J=5.5 Hz, 1H), 7.82 (d, J=8.5 Hz, 1H), 7.45 (d, J=17.5 Hz, 1H), 7.31 (d, J=7.5 Hz, 1H), 7.13 (t, J=54.1 Hz, 1H), 6.67 (dd, J=163.9, 7.8 Hz, 1H), 5.18 (d, J=81.6 Hz, 1H), 4.77 (d, J=20.0 Hz, 1H), 4.22 (s, 3H), 4.09-3.71 (m, 6H), 3.67-3.55 (m, 3H), 3.49-3.39 (m, 2H), 3.05 (d, J=56.0 Hz, 2H), 2.84 (t, J=6.8 Hz, 2H), 2.23 (s, 2H), 2.16-1.83 (m, 10H), 1.26 (d, J=12.1 Hz, 2H). LCMS (ESI) calcd, for C₄₁H₄₅F₂N₁₂O₆⁺ [M+H]⁺: 839.36, found, 839.4.

### Example 126: Preparation of 5-((1R,4R)-2-oxa-5-azabicyclo[2.2.1]heptan-5-yl)-N-(3-(difluoromethyl)-1-((1r,4R)-4-((5-(2-(2,4-dioxotetrahydropyrimidin-1(2H)-yl)-1,3-dioxoisoindolin-5-yl)-2,5-diazabicyclo[2.2.2]octan-2-yl)methyl)cyclohexyl)-1H-pyrazol-4-yl)pyrazolo[1,5-a]pyrimidine-3-carboxamide (GT-05833)

With reference to the method of Scheme T-3, the target compound (GT-05833) was obtained (yellow solid, 29 mg, yield 41.96%). ¹H NMR (400 MHz, DMSO-d₆) δ 10.84 (s, 1H), 9.88 (s, 1H), 9.51 (d, J=4.9 Hz, 1H), 8.78 (d, J=7.4 Hz, 1H), 8.40 (s, 1H), 8.25 (d, J=4.3 Hz, 1H), 7.78 (d, J=6.9 Hz, 1H), 7.33-6.96 (m, 3H), 6.66 (dd, J=163.5, 7.7 Hz, 1H), 5.17 (d, J=81.8 Hz, 1H), 4.77 (d, J=20.8 Hz, 1H), 4.59 (s, 1H), 4.23 (s, 1H), 4.14-3.94 (m, 2H), 3.76 (dd, J=24.7, 7.4 Hz, 5H), 3.60 (dd, J=28.2, 16.7 Hz, 3H), 3.45 (d, J=8.2 Hz, 2H), 3.22 (d, J=22.4 Hz, 4H), 2.84 (d, J=6.1 Hz, 2H), 2.15-1.77 (m, 11H), 1.20 (d, J=13.1 Hz, 1H). LCMS (ESI) calcd, for C₄₁H₄₅F₂N₁₂O₆⁺ [M+H]⁺: 839.36, found, 839.4.

### Example 127: Preparation of N-(2-((1r,4r)-4-((4-(2-(2,4-dioxotetrahydropyrimidin-1(2H)-yl)-1,3-dioxoisoindolin-5-yl)piperazin-1-yl)methyl)cyclohexyl)-6-methoxy-2H-indazol-5-yl)-6-(trifluoromethyl) picolinamide (GT-08647)

With reference to the method of Scheme T-3, the target compound (GT-08647) was obtained (yellow solid, 25 mg, yield 36.06%). ¹H NMR (400 MHz, DMSO) § 10.86 (s, 1H), 10.51 (s, 1H), 10.35 (s, 1H), 8.70 (s, 1H), 8.44 (dd, J=18.0, 7.7 Hz, 2H), 8.35 (s, 1H), 8.22 (d, J=7.8 Hz, 1H), 7.83 (d, J=8.5 Hz, 1H), 7.56 (s, 1H), 7.48-7.38 (m, 1H), 7.15 (s, 1H), 4.41 (d, J=11.6 Hz, 1H), 4.24 (d, J=13.3 Hz, 2H), 3.99 (s, 3H), 3.80 (t, J=6.8 Hz, 2H), 3.64 (d, J=11.7 Hz, 2H), 3.54 (s, 2H), 3.13 (d, J=22.9 Hz, 4H), 2.84 (t, J=6.9 Hz, 2H), 2.18 (d, J=10.5 Hz, 2H), 2.02 (dd, J=38.9, 12.8 Hz, 5H), 1.28 (d, J=13.0 Hz, 2H). LCMS (ESI) calcd, for C₃₈H₃₉F₃N₉O₆⁺ [M+H]⁺: 774.30, found, 774.3.

### Example 128: Preparation of N-(2-((1S,4r)-4-(((1S,4S)-5-(2-(2,4-dioxotetrahydropyrimidin-1(2H)-yl)-1,3-dioxoisoindolin-5-yl)-2,5-diazabicyclo[2.2.1]heptan-2-yl)methyl)cyclohexyl)-6-methoxy-2H-indazol-5-yl)-6-(trifluoromethyl) picolinamide (GT-08648)

With reference to the method of Scheme T-3, the target compound (GT-08648) was obtained (yellow solid, 37 mg, yield 52.55%). ¹H NMR (400 MHz, DMSO) δ 11.11 (s, 1H), 10.85 (s, 1H), 9.85 (s, 1H), 8.70 (d, J=5.9 Hz, 1H), 8.50-8.38 (m, 2H), 8.34 (s, 1H), 8.25-8.19 (m, 1H), 7.77 (d, J=8.4 Hz, 1H), 7.23 (s, 1H), 7.15 (s, 1H), 7.02 (s, 1H), 5.03 (s, 1H), 4.58 (s, 1H), 4.40 (s, 1H), 3.98 (s, 3H), 3.85-3.69 (m, 4H), 3.32 (d, J=11.3 Hz, 1H), 3.09 (d, J=34.1 Hz, 2H), 2.84 (t, J=6.5 Hz, 2H), 2.45 (s, 2H), 2.28-1.85 (m, 8H), 1.24 (d, J=8.2 Hz, 2H). LCMS (ESI) calcd, for C₃₉H₃₉F₃N₉O₆⁺ [M+H]⁺: 786.30, found, 786.3.

### Example 129: Preparation of N-(2-((1R,4r)-4-(((1R,4R)-5-(2-(2,4-dioxotetrahydropyrimidin-1(2H)-yl)-1,3-dioxoisoindolin-5-yl)-2,5-diazabicyclo[2.2.1]heptan-2-yl)methyl)cyclohexyl)-6-methoxy-2H-indazol-5-yl)-6-(trifluoromethyl) picolinamide (GT-08650)

With reference to the method of Scheme T-3, the target compound (GT-08650) was obtained (yellow solid, 32 mg, yield 45.45%). ¹H NMR (400 MHz, DMSO) δ 11.12 (s, 1H), 10.85 (s, 1H), 10.50 (s, 1H), 9.87 (s, 1H), 8.70 (d, J=5.8 Hz, 1H), 8.51-8.37 (m, 2H), 8.34 (s, 1H), 8.22 (d, J=7.6 Hz, 1H), 7.78 (t, J=6.9 Hz, 1H), 7.23 (s, 1H), 7.15 (s, 1H), 7.02 (s, 1H), 5.00 (d, J=19.7 Hz, 1H), 4.65 (d, J=52.2 Hz, 1H), 4.39 (d, J=11.6 Hz, 1H), 3.98 (s, 3H), 3.85-3.69 (m, 4H), 3.56 (s, 1H), 3.31 (d, J=9.0 Hz, 1H), 3.07 (d, J=47.8 Hz, 2H), 2.89-2.76 (m, 2H), 2.45 (s, 1H), 2.28-1.84 (m, 8H), 1.24 (d, J=8.1 Hz, 2H). LCMS (ESI) calcd, for C₃₉H₃₉F₃N₉O₆⁺ [M+H]⁺: 786.30, found, 786.3.

### Example 130: Preparation of N-(2-((1r,4r)-4-((4-(2-(2,4-dioxotetrahydropyrimidin-1(2H)-yl)-6-fluoro-1,3-dioxoisoindolin-5-yl)piperazin-1-yl)methyl)cyclohexyl)-6-methoxy-2H-indazol-5-yl)-6-(trifluoromethyl) picolinamide (GT-08651)

With reference to the method of Scheme T-3, the target compound (GT-08651) was obtained (yellow solid, 38 mg, yield 53.57%). ¹H NMR (400 MHz, DMSO) δ 10.90 (s, 1H), 10.51 (s, 1H), 10.02 (s, 1H), 8.70 (s, 1H), 8.44 (dd, J=18.2, 7.6 Hz, 2H), 8.35 (s, 1H), 8.22 (d, J=7.7 Hz, 1H), 7.91 (d, J=11.3 Hz, 1H), 7.68 (d, J=7.5 Hz, 1H), 7.15 (s, 1H), 4.42 (d, J=11.7 Hz, 1H), 3.99 (s, 3H), 3.81 (t, J=6.8 Hz, 3H), 3.65 (d, J=9.6 Hz, 2H), 3.48 (s, 3H), 3.28-3.23 (m, 2H), 3.13 (s, 2H), 2.85 (t, J=6.8 Hz, 2H), 2.19 (d, J=11.4 Hz, 2H), 2.00 (dd, J=28.4, 12.9 Hz, 5H), 1.30 (d, J=12.8 Hz, 2H). LCMS (ESI) calcd, for C₃₈H₃₈F₄N₉O₆⁺ [M+H]⁺: 792.29, found, 792.3.

### Example 131: Preparation of N-(2-((1r,4r)-4-((8-(2-(2,4-dioxotetrahydropyrimidin-1(2H)-yl)-1,3-dioxoisoindolin-5-yl)-3,8-diazabicyclo[3.2.1]octan-3-yl)methyl)cyclohexyl)-6-methoxy-2H-indazol-5-yl)-6-(trifluoromethyl) picolinamide (GT-08652)

With reference to the method of Scheme T-3, the target compound (GT-08652) was obtained (yellow solid, 42 mg, yield 58.61%). ¹H NMR (400 MHz, DMSO) δ 10.86 (s, 1H), 10.50 (s, 1H), 10.08 (s, 1H), 8.69 (s, 1H), 8.44 (dd, J=17.8, 7.7 Hz, 2H), 8.32 (s, 1H), 8.22 (d, J=7.6 Hz, 1H), 7.83 (d, J=8.5 Hz, 1H), 7.47 (s, 1H), 7.33 (d, J=8.7 Hz, 1H), 7.14 (s, 1H), 4.80 (s, 2H), 4.37 (t, J=11.3 Hz, 1H), 3.98 (s, 3H), 3.80 (t, J=6.8 Hz, 2H), 3.43 (s, 2H), 3.09 (d, J=11.0 Hz, 2H), 2.93 (s, 2H), 2.85 (t, J=7.3 Hz, 2H), 2.38 (d, J=7.7 Hz, 2H), 2.11 (s, 4H), 1.94 (dd, J=26.2, 12.9 Hz, 5H), 1.24 (d, J=11.5 Hz, 2H). LCMS (ESI) calcd, for C₄₀H₄₁F₃N₉O₆⁺ [M+H]⁺: 800.31, found, 800.3.

### Example 132: Preparation of N-(2-((1r,4r)-4-((3-(2-(2,4-dioxotetrahydropyrimidin-1(2H)-yl)-1,3-dioxoisoindolin-5-yl)-3,6-diazabicyclo[3.1.1]heptan-6-yl)methyl)cyclohexyl)-6-methoxy-2H-indazol-5-yl)-6-(trifluoromethyl) picolinamide (GT-08653)

With reference to the method of Scheme T-3, the target compound (GT-08653) was obtained (yellow solid, 23 mg, yield 32.67%). ¹H NMR (400 MHz, DMSO) δ 11.02 (s, 1H), 10.86 (s, 1H), 10.51 (s, 1H), 8.74-8.65 (m, 1H), 8.52-8.38 (m, 2H), 8.35 (d, J=4.8 Hz, 1H), 8.22 (d, J=7.6 Hz, 1H), 7.91-7.82 (m, 1H), 7.28-7.12 (m, 2H), 4.69 (s, 1H), 4.57-4.32 (m, 2H), 4.13-3.94 (m, 6H), 3.81 (t, J=6.0 Hz, 2H), 3.50 (s, 2H), 3.06 (d, J=36.7 Hz, 1H), 2.87-2.76 (m, 3H), 2.15 (d, J=14.9 Hz, 2H), 1.94 (dd, J=35.1, 10.2 Hz, 5H), 1.30 (s, 2H). LCMS (ESI) calcd, for C₃₉H₃₉F₃N₉O₆⁺ [M+H]⁺: 786.30, found, 786.3.

### Example 133: Preparation of N-(2-((1r,4r)-4-((3-(2-(2,4-dioxotetrahydropyrimidin-1(2H)-yl)-1,3-dioxoisoindolin-5-yl)-3,8-diazabicyclo[3.2.1]octan-8-yl)methyl)cyclohexyl)-6-methoxy-2H-indazol-5-yl)-6-(trifluoromethyl) picolinamide (GT-08654)

With reference to the method of Scheme T-3, the target compound (GT-08654) was obtained (yellow solid, mg, yield %). ¹H NMR (400 MHz, DMSO) δ 10.87 (s, 1H), 10.79 (s, 1H), 10.48 (d, J=28.7 Hz, 1H), 8.71 (s, 1H), 8.54-8.34 (m, 3H), 8.22 (d, J=7.8 Hz, 1H), 7.83 (t, J=7.5 Hz, 1H), 7.45 (t, J=7.4 Hz, 1H), 7.31 (d, J=6.9 Hz, 1H), 7.16 (s, 1H), 4.44 (t, J=11.6 Hz, 1H), 4.23 (s, 2H), 4.02 (d, J=21.7 Hz, 5H), 3.85-3.73 (m, 4H), 3.59 (s, 1H), 3.00 (s, 2H), 2.84 (t, J=7.0 Hz, 2H), 2.18 (dd, J=29.9, 14.4 Hz, 6H), 1.98 (d, J=9.0 Hz, 4H), 1.41-1.24 (m, 2H). LCMS (ESI) calcd, for C₄₀H₄₁F₃N₉O₆⁺ [M+H]⁺: 800.31, found, 800.3.

### Example 134: Preparation of N-(2-((1r,4r)-4-((4-(2-(2,4-dioxotetrahydropyrimidin-1(2H)-yl)-1,3-dioxoisoindolin-4-yl)piperazin-1-yl)methyl)cyclohexyl)-6-methoxy-2H-indazol-5-yl)-6-(trifluoromethyl) picolinamide (GT-08655)

With reference to the method of Scheme T-3, the target compound (GT-08655) was obtained (yellow solid, 40 mg, yield 57.70%). ¹H NMR (400 MHz, DMSO) δ 10.89 (s, 1H), 10.51 (s, 1H), 10.41 (s, 1H), 8.70 (s, 1H), 8.43 (d, J=18.5 Hz, 2H), 8.35 (s, 1H), 8.22 (dd, J=7.6, 0.9 Hz, 1H), 7.88-7.80 (m, 1H), 7.51 (dd, J=9.7, 8.0 Hz, 2H), 7.16 (s, 1H), 4.43 (t, J=11.8 Hz, 1H), 3.99 (s, 3H), 3.90 (d, J=12.3 Hz, 1H), 3.85-3.77 (m, 3H), 3.71 (s, 2H), 3.55 (dd, J=24.1, 12.1 Hz, 2H), 3.27 (d, J=10.9 Hz, 2H), 3.14 (s, 2H), 2.83 (s, 2H), 2.18 (d, J=9.8 Hz, 2H), 2.01 (dt, J=24.4, 12.7 Hz, 5H), 1.30 (dd, J=23.0, 11.1 Hz, 2H). LCMS (ESI) calcd. for C₃₈H₃₉F₃N₉O₆⁺ [M+H]⁺: 774.30, found, 774.3.

### Example 135: Preparation of N-(2-((1r,4r)-4-((4-(2-(2,4-dioxotetrahydropyrimidin-1(2H)-yl)-4-fluoro-1,3-dioxoisoindolin-5-yl)piperazin-1-yl)methyl)cyclohexyl)-6-methoxy-2H-indazol-5-yl)-6-(trifluoromethyl) picolinamide (GT-08656)

With reference to the method of Scheme T-3, the target compound (GT-08656) was obtained (yellow solid, 34 mg, yield 47.93%). ¹H NMR (400 MHz, DMSO) δ 10.87 (d, J=30.0 Hz, 1H), 10.51 (d, J=4.0 Hz, 2H), 8.70 (s, 1H), 8.49-8.38 (m, 2H), 8.34 (d, J=4.8 Hz, 1H), 8.22 (dd, J=7.7, 1.0 Hz, 1H), 7.77 (d, J=8.1 Hz, 1H), 7.56 (t, J=7.9 Hz, 1H), 7.16 (s, 1H), 4.43 (t, J=11.8 Hz, 1H), 3.99 (s, 3H), 3.80 (t, J=6.8 Hz, 3H), 3.64 (dd, J=16.3, 7.7 Hz, 3H), 3.50 (s, 3H), 3.26 (d, J=9.7 Hz, 2H), 3.12 (s, 2H), 2.90-2.74 (m, 2H), 2.18 (d, J=10.0 Hz, 2H), 2.08 (d, J=13.1 Hz, 2H), 1.96 (dd, J=23.1, 11.2 Hz, 3H), 1.29 (dd, J=22.9, 11.7 Hz, 2H). LCMS (ESI) calcd, for C₃₈H₃₈F₄N₉O₆⁺ [M+H]⁺: 792.29, found, 792.3.

### Example 136: Preparation of N-(2-((1r,4r)-4-((4-(2-(2,4-dioxotetrahydropyrimidin-1(2H)-yl)-7-fluoro-1,3-dioxoisoindolin-5-yl)piperazin-1-yl)methyl)cyclohexyl)-6-methoxy-2H-indazol-5-yl)-6-(trifluoromethyl) picolinamide (GT-08657)

With reference to the method of Scheme T-3, the target compound (GT-08657) was obtained (yellow solid, 41 mg, yield 57.80%). ¹H NMR (400 MHz, DMSO) δ 10.88 (s, 1H), 10.51 (s, 1H), 10.42 (s, 1H), 8.70 (s, 1H), 8.51-8.38 (m, 2H), 8.35 (s, 1H), 8.22 (dd, J=7.6, 1.0 Hz, 1H), 7.46 (d, J=1.6 Hz, 1H), 7.27 (d, J=13.1 Hz, 1H), 7.15 (s, 1H), 4.42 (t, J=11.7 Hz, 1H), 4.29 (s, 2H), 3.99 (s, 3H), 3.79 (td, J=6.7, 1.7 Hz, 2H), 3.60 (t, J=12.6 Hz, 4H), 3.11 (d, J=20.2 Hz, 4H), 2.84 (t, J=7.5 Hz, 2H), 2.18 (d, J=9.9 Hz, 2H), 2.02 (dd, J=39.1, 12.2 Hz, 5H), 1.27 (d, J=12.5 Hz, 2H). LCMS (ESI) calcd, for C₃₈H₃₈F₄N₉O₆⁺ [M+H]⁺: 792.29, found, 792.3.

### Example 137: Preparation of N-(2-((1r,4r)-4-((4-(1-(2-(2,4-dioxotetrahydropyrimidin-1(2H)-yl)-1,3-dioxoisoindolin-5-yl)piperidin-4-yl)piperazin-1-yl)methyl)cyclohexyl)-6-methoxy-2H-indazol-5-yl)-6-(trifluoromethyl) picolinamide (GT-08658)

With reference to the method of Scheme T-3, the target compound (GT-08658) was obtained (yellow solid, 37 mg, yield 48.19%). ¹H NMR (400 MHz, DMSO) δ 12.16 (s, 1H), 11.33 (s, 1H), 10.85 (s, 1H), 10.51 (s, 1H), 8.70 (s, 1H), 8.50-8.38 (m, 2H), 8.34 (s, 1H), 8.22 (d, J=7.7 Hz, 1H), 7.76 (d, J=8.6 Hz, 1H), 7.49 (s, 1H), 7.37 (d, J=8.6 Hz, 1H), 7.15 (s, 1H), 4.48-4.27 (m, 3H), 3.99 (s, 3H), 3.77 (dd, J=21.9, 15.0 Hz, 8H), 3.51 (s, 3H), 3.02 (t, J=12.5 Hz, 4H), 2.84 (t, J=7.1 Hz, 2H), 2.28-2.06 (m, 6H), 2.02-1.88 (m, 3H), 1.78 (s, 2H), 1.25 (d, J=10.9 Hz, 2H). LCMS (ESI) calcd, for C₄₃H₄₈F₃N₁₀O₆⁺ [M+H]⁺: 857.37, found, 857.5.

### Example 138: Preparation of N-(2-((1r,4r)-4-((4-(1-(2-(2,4-dioxotetrahydropyrimidin-1(2H)-yl)-1,3-dioxoisoindolin-5-yl)-3,3-difluoropiperidin-4-yl)piperazin-1-yl)methyl)cyclohexyl)-6-methoxy-2H-indazol-5-yl)-6-(trifluoromethyl) picolinamide (GT-08659)

With reference to the method of Scheme T-3, the target compound (GT-08659) was obtained (yellow solid, 12 mg, yield 15.00%). ¹H NMR (400 MHz, DMSO) δ 10.85 (s, 1H), 10.51 (s, 1H), 9.63 (s, 1H), 8.70 (s, 1H), 8.44 (dd, J=17.7, 7.8 Hz, 2H), 8.34 (s, 1H), 8.22 (d, J=7.6 Hz, 1H), 7.63 (d, J=87.1 Hz, 2H), 7.14 (s, 1H), 4.35 (dd, J=73.4, 23.2 Hz, 3H), 3.98 (s, 3H), 3.79 (t, J=6.8 Hz, 2H), 3.49 (s, 2H), 3.37 (s, 2H), 3.03 (d, J=6.9 Hz, 9H), 2.83 (t, J=6.9 Hz, 2H), 2.16 (d, J=11.6 Hz, 2H), 2.03-1.84 (m, 7H), 1.25 (d, J=13.2 Hz, 3H). LCMS (ESI) calcd, for C₄₃H₄₆F₅N₁₀O₆⁺ [M+H]⁺: 893.35, found, 893.5.

### Example 139: Preparation of N-(2-((1r,4r)-4-((5-(2-(2,4-dioxotetrahydropyrimidin-1(2H)-yl)-1,3-dioxoisoindolin-5-yl)-2,5-diazabicyclo[2.2.2]octan-2-yl)methyl)cyclohexyl)-6-methoxy-2H-indazol-5-yl)-6-(trifluoromethyl) picolinamide (GT-08660)

With reference to the method of Scheme T-3, the target compound (GT-08660) was obtained (yellow solid, 30 mg, yield 41.86%). ¹H NMR (400 MHz, DMSO) δ 10.85 (s, 1H), 10.51 (s, 1H), 10.44 (s, 1H), 8.70 (s, 1H), 8.50-8.38 (m, 2H), 8.35 (s, 1H), 8.22 (d, J=7.8 Hz, 1H), 7.79 (t, J=7.4 Hz, 1H), 7.25 (d, J=9.2 Hz, 1H), 7.15 (s, 1H), 7.09 (s, 1H), 4.61 (s, 1H), 4.40 (d, J=8.4 Hz, 1H), 4.28-4.06 (m, 1H), 3.99 (s, 4H), 3.87 (s, 1H), 3.79 (t, J=6.7 Hz, 2H), 3.63 (d, J=11.8 Hz, 2H), 3.24 (d, J=13.1 Hz, 3H), 2.84 (t, J=6.8 Hz, 2H), 2.33 (s, 1H), 2.22-2.07 (m, 4H), 2.01-1.86 (m, 5H), 1.37-1.17 (m, 2H). LCMS (ESI) calcd, for C₄₀H₄₁F₃N₉O₆⁺ [M+H]⁺: 800.3, found, 800.3.

### Example 140: Preparation of N-(2-((1r,4r)-4-((4-(2-(2,4-dioxotetrahydropyrimidin-1(2H)-yl)-1,3-dioxoisoindolin-5-yl)piperazin-1-yl)methyl)cyclohexyl)-6-methoxy-2H-indazol-5-yl)-3-(trifluoromethyl)benzamide (GT-08661)

With reference to the method of Scheme T-3, the target compound (GT-08661) was obtained (yellow solid, 36 mg, yield 51.87%). ¹H NMR (400 MHz, DMSO) δ 10.86 (s, 1H), 10.43 (s, 1H), 9.78 (s, 1H), 8.34-8.25 (m, 3H), 8.02 (s, 1H), 7.96 (s, 1H), 7.86-7.75 (m, 2H), 7.57 (d, J=2.0 Hz, 1H), 7.42 (dd, J=8.7, 2.1 Hz, 1H), 7.08 (s, 1H), 4.43 (t, J=11.6 Hz, 1H), 4.24 (d, J=12.8 Hz, 2H), 3.87 (s, 3H), 3.80 (t, J=6.8 Hz, 2H), 3.67-3.53 (m, 4H), 3.10 (s, 4H), 2.84 (t, J=7.0 Hz, 2H), 2.17 (s, 2H), 2.02 (d, J=27.4 Hz, 5H), 1.28 (dd, J=23.4, 10.3 Hz, 2H). LCMS (ESI) calcd, for C₃₉H₄₀F₃N₈O₆⁺ [M+H]⁺: 773.30, found, 773.3.

### Example 141: Preparation of N-(2-((1S,4r)-4-(((1S,4S)-5-(2-(2,4-dioxotetrahydropyrimidin-1(2H)-yl)-1,3-dioxoisoindolin-5-yl)-2,5-diazabicyclo[2.2.1]heptan-2-yl)methyl)cyclohexyl)-6-methoxy-2H-indazol-5-yl)-3-(trifluoromethyl)benzamide (GT-08662)

With reference to the method of Scheme T-3, the target compound (GT-08662) was obtained (yellow solid, 42 mg, yield 59.60%). ¹H NMR (400 MHz, DMSO) δ 11.02 (s, 1H), 10.85 (s, 1H), 9.77 (s, 2H), 8.29 (dd, J=12.8, 6.0 Hz, 3H), 8.08-7.90 (m, 2H), 7.78 (t, J=7.9 Hz, 2H), 7.23 (s, 1H), 7.06 (s, 2H), 5.03 (s, 1H), 4.58 (s, 1H), 4.40 (d, J=11.3 Hz, 1H), 3.96 (d, J=11.7 Hz, 1H), 3.87 (s, 3H), 3.84-3.68 (m, 4H), 3.32 (d, J=10.7 Hz, 1H), 3.14 (s, 2H), 2.84 (t, J=6.7 Hz, 2H), 2.43 (d, J=12.7 Hz, 2H), 2.22-1.88 (m, 7H), 1.24 (s, 2H). LCMS (ESI) calcd, for C₄₀H₄₀F₃N₈O₆⁺ [M+H]⁺: 785.30, found, 785.3.

### Example 142: Preparation of N-(2-((1R,4r)-4-(((1R,4R)-5-(2-(2,4-dioxotetrahydropyrimidin-1(2H)-yl)-1,3-dioxoisoindolin-5-yl)-2,5-diazabicyclo[2.2.1]heptan-2-yl)methyl)cyclohexyl)-6-methoxy-2H-indazol-5-yl)-3-(trifluoromethyl)benzamide (GT-08663)

With reference to the method of Scheme T-3, the target compound (GT-08663) was obtained (yellow solid, 34 mg, yield 51.09%). ¹H NMR (400 MHz, DMSO) δ 11.04 (s, 1H), 10.85 (s, 1H), 9.77 (s, 2H), 8.29 (dd, J=12.9, 6.2 Hz, 3H), 8.06-7.92 (m, 2H), 7.78 (t, J=7.9 Hz, 2H), 7.23 (s, 1H), 7.06 (s, 2H), 5.00 (d, J=20.4 Hz, 1H), 4.65 (d, J=53.7 Hz, 1H), 4.41 (t, J=11.6 Hz, 1H), 3.97 (d, J=11.1 Hz, 1H), 3.87 (s, 3H), 3.78 (dd, J=14.5, 7.9 Hz, 4H), 3.32 (d, J=11.1 Hz, 1H), 3.11 (dd, J=23.0, 16.1 Hz, 2H), 2.85 (d, J=6.5 Hz, 2H), 2.43 (d, J=12.1 Hz, 1H), 2.03 (d, J=93.8 Hz, 8H), 1.23 (s, 2H). LCMS (ESI) calcd, for C₄₀H₄₀F₃N₈O₆⁺ [M+H]⁺: 785.30, found, 785.3.

### Example 143: Preparation of N-(2-((1r,4r)-4-((4-(2-(2,4-dioxotetrahydropyrimidin-1(2H)-yl)-6-fluoro-1,3-dioxoisoindolin-5-yl)piperazin-1-yl)methyl)cyclohexyl)-6-methoxy-2H-indazol-5-yl)-3-(trifluoromethyl)benzamide (GT-08664)

With reference to the method of Scheme T-3, the target compound (GT-08664) was obtained (yellow solid, 37 mg, yield 52.10%). ¹H NMR (400 MHz, DMSO) δ 10.90 (s, 1H), 10.59 (s, 1H), 9.78 (s, 1H), 8.29 (dd, J=13.4, 7.0 Hz, 3H), 8.02 (s, 1H), 7.97 (d, J=7.7 Hz, 1H), 7.91 (d, J=11.4 Hz, 1H), 7.79 (t, J=7.8 Hz, 1H), 7.67 (d, J=7.5 Hz, 1H), 7.08 (s, 1H), 4.43 (s, 1H), 3.95-3.79 (m, 7H), 3.61 (dd, J=24.8, 12.0 Hz, 4H), 3.25 (s, 2H), 3.11 (s, 2H), 2.85 (t, J=6.8 Hz, 2H), 2.27-1.93 (m, 7H), 1.29 (d, J=12.1 Hz, 2H). LCMS (ESI) calcd. for C₃₉H₃₉F₄N₈O₆⁺ [M+H]⁺: 791.29, found, 791.3.

### Example 144: Preparation of N-(2-((1r,4r)-4-((8-(2-(2,4-dioxotetrahydropyrimidin-1(2H)-yl)-1,3-dioxoisoindolin-5-yl)-3,8-diazabicyclo[3.2.1]octan-3-yl)methyl)cyclohexyl)-6-methoxy-2H-indazol-5-yl)-3-(trifluoromethyl)benzamide (GT-08665)

With reference to the method of Scheme T-3, the target compound (GT-08665) was obtained (yellow solid, 45 mg, yield 62.74%). ¹H NMR (400 MHz, DMSO) δ 10.86 (s, 1H), 10.19 (s, 1H), 9.78 (s, 1H), 8.29 (s, 3H), 8.01 (s, 1H), 7.97 (d, J=7.7 Hz, 1H), 7.84-7.74 (m, 2H), 7.47 (d, J=1.6 Hz, 1H), 7.41-7.28 (m, 1H), 7.06 (s, 1H), 4.81 (s, 2H), 4.37 (t, J=11.8 Hz, 1H), 3.87 (s, 3H), 3.80 (t, J=6.8 Hz, 2H), 3.44 (d, J=10.9 Hz, 2H), 3.16-3.03 (m, 2H), 2.92 (s, 2H), 2.85 (d, J=1.6 Hz, 2H), 2.40 (d, J=7.8 Hz, 2H), 2.11 (s, 4H), 1.96 (s, 5H), 1.24 (d, J=12.4 Hz, 2H). LCMS (ESI) calcd, for C₄₁H₄₂F₃N₈O₆⁺ [M+H]⁺: 799.32, found, 799.3.

### Example 145: Preparation of N-(2-((1r,4r)-4-((3-(2-(2,4-dioxotetrahydropyrimidin-1(2H)-yl)-1,3-dioxoisoindolin-5-yl)-3,6-diazabicyclo[3.1.1]heptan-6-yl)methyl)cyclohexyl)-6-methoxy-2H-indazol-5-yl)-3-(trifluoromethyl)benzamide (GT-08666)

With reference to the method of Scheme T-3, the target compound (GT-08666) was obtained (yellow solid, 42 mg, yield 59.60%). ¹H NMR (400 MHz, DMSO) δ 11.15 (s, 1H), 10.86 (s, 1H), 9.78 (s, 1H), 8.37-8.23 (m, 3H), 7.99 (dd, J=16.9, 5.6 Hz, 2H), 7.86 (dd, J=8.5, 6.7 Hz, 1H), 7.78 (t, J=7.7 Hz, 1H), 7.27 (dd, J=10.1, 2.0 Hz, 1H), 7.19 (d, J=8.7 Hz, 1H), 7.07 (s, 1H), 4.68 (s, 1H), 4.53 (d, J=6.0 Hz, 2H), 4.14-3.97 (m, 3H), 3.87 (s, 3H), 3.81 (dd, J=6.7, 4.8 Hz, 2H), 3.55 (s, 2H), 3.43 (s, 1H), 2.91-2.81 (m, 3H), 2.25-2.11 (m, 2H), 1.94 (dd, J=31.2, 9.8 Hz, 6H), 1.38-1.20 (m, 2H). LCMS (ESI) calcd, for C₄₀H₄₀F₃N₈O₆⁺ [M+H]⁺: 785.30, found, 785.3.

### Example 146: Preparation of N-(2-((1r,4r)-4-((3-(2-(2,4-dioxotetrahydropyrimidin-1(2H)-yl)-1,3-dioxoisoindolin-5-yl)-3,8-diazabicyclo[3.2.1]octan-8-yl)methyl)cyclohexyl)-6-methoxy-2H-indazol-5-yl)-3-(trifluoromethyl)benzamide (GT-08667)

With reference to the method of Scheme T-3, the target compound (GT-08667) was obtained (yellow solid, 41 mg, yield 59.95%). ¹H NMR (400 MHz, DMSO) § 10.86 (s, 1H), 10.41 (s, 1H), 9.78 (s, 1H), 8.39-8.25 (m, 3H), 8.02 (s, 1H), 7.97 (d, J=7.6 Hz, 1H), 7.88-7.73 (m, 2H), 7.47 (s, 1H), 7.31 (d, J=6.7 Hz, 1H), 7.08 (s, 1H), 4.44 (s, 1H), 4.23 (s, 2H), 4.04 (d, J=12.7 Hz, 2H), 3.88 (s, 3H), 3.79 (t, J=6.8 Hz, 2H), 3.70 (d, J=12.3 Hz, 2H), 3.00 (s, 2H), 2.84 (t, J=6.9 Hz, 2H), 2.27-1.95 (m, 11H), 1.46-1.23 (m, 2H). LCMS (ESI) calcd, for C₄₁H₄₂F₃N₈O₆⁺ [M+H]⁺: 799.32, found, 799.3.

### Example 147: Preparation of N-(2-((1r,4r)-4-((4-(2-(2,4-dioxotetrahydropyrimidin-1(2H)-yl)-1,3-dioxoisoindolin-4-yl)piperazin-1-yl)methyl)cyclohexyl)-6-methoxy-2H-indazol-5-yl)-3-(trifluoromethyl)benzamide (GT-08668)

With reference to the method of Scheme T-3, the target compound (GT-08668) was obtained (yellow solid, 43 mg, yield 61.96%). ¹H NMR (400 MHz, DMSO) δ 10.89 (s, 1H), 10.71 (s, 1H), 9.79 (s, 1H), 8.30 (dd, J=13.6, 7.1 Hz, 3H), 8.02 (s, 1H), 7.97 (d, J=7.8 Hz, 1H), 7.81 (dt, J=17.6, 7.6 Hz, 2H), 7.55-7.45 (m, 2H), 7.09 (s, 1H), 4.44 (t, J=11.8 Hz, 1H), 3.89 (d, J=13.3 Hz, 4H), 3.80 (t, J=6.7 Hz, 3H), 3.70 (s, 2H), 3.60 (d, J=12.8 Hz, 2H), 3.25 (dd, J=18.1, 10.7 Hz, 2H), 3.13 (s, 2H), 2.98-2.80 (m, 2H), 2.15 (dd, J=27.8, 11.3 Hz, 4H), 1.97 (dd, J=24.5, 12.0 Hz, 3H), 1.30 (dd, J=23.5, 11.5 Hz, 2H). LCMS (ESI) calcd, for C₃₉H₄₀F₃N₈O₆⁺ [M+H]⁺: 773.30, found, 773.3.

### Example 148: Preparation of N-(2-((1r,4r)-4-((4-(2-(2,4-dioxotetrahydropyrimidin-1(2H)-yl)-4-fluoro-1,3-dioxoisoindolin-5-yl)piperazin-1-yl)methyl)cyclohexyl)-6-methoxy-2H-indazol-5-yl)-3-(trifluoromethyl)benzamide (GT-08669)

With reference to the method of Scheme T-3, the target compound (GT-08669) was obtained (yellow solid, 35 mg, yield 49.29%). ¹H NMR (400 MHz, DMSO) δ 10.91 (s, 1H), 10.52 (s, 1H), 9.78 (s, 1H), 8.29 (dd, J=13.4, 6.7 Hz, 3H), 8.02 (s, 1H), 7.97 (d, J=7.8 Hz, 1H), 7.78 (t, J=8.1 Hz, 2H), 7.55 (t, J=7.9 Hz, 1H), 7.08 (s, 1H), 4.43 (s, 1H), 3.87 (s, 3H), 3.80 (t, J=6.8 Hz, 4H), 3.66 (d, J=10.8 Hz, 2H), 3.58-3.49 (m, 2H), 3.26 (d, J=8.9 Hz, 2H), 3.12 (s, 2H), 2.84 (t, J=6.8 Hz, 2H), 2.18 (d, J=10.1 Hz, 2H), 2.08 (d, J=12.0 Hz, 2H), 2.03-1.90 (m, 3H), 1.29 (d, J=12.1 Hz, 2H). LCMS (ESI) calcd, for C₃₉H₃₉F₄N₈O₆⁺ [M+H]⁺: 791.29, found, 791.3.

### Example 149: Preparation of N-(2-((1r,4r)-4-((4-(2-(2,4-dioxotetrahydropyrimidin-1(2H)-yl)-7-fluoro-1,3-dioxoisoindolin-5-yl)piperazin-1-yl)methyl)cyclohexyl)-6-methoxy-2H-indazol-5-yl)-3-(trifluoromethyl)benzamide (GT-08670)

With reference to the method of Scheme T-3, the target compound (GT-08670) was obtained (yellow solid, 44 mg, yield 61.96%). ¹H NMR (400 MHz, DMSO) δ 10.88 (s, 1H), 10.58 (s, 1H), 9.78 (s, 1H), 8.29 (dd, J=13.0, 6.4 Hz, 3H), 8.02 (s, 1H), 7.97 (d, J=8.0 Hz, 1H), 7.79 (t, J=7.8 Hz, 1H), 7.45 (s, 1H), 7.27 (d, J=12.0 Hz, 1H), 7.08 (s, 1H), 4.43 (t, J=11.6 Hz, 1H), 4.29 (s, 2H), 3.87 (s, 3H), 3.80 (dt, J=8.2, 5.9 Hz, 2H), 3.63 (t, J=10.5 Hz, 4H), 3.10 (d, J=20.2 Hz, 4H), 2.84 (t, J=7.4 Hz, 2H), 2.18 (d, J=10.1 Hz, 2H), 2.08 (d, J=12.4 Hz, 2H), 1.97 (dd, J=23.3, 11.2 Hz, 3H), 1.27 (dd, J=22.6, 10.2 Hz, 2H). LCMS (ESI) calcd. for C₃₉H₃₉F₄N₈O₆⁺ [M+H]⁺: 791.29, found, 791.3.

### Example 150: Preparation of N-(2-((1r,4r)-4-((4-(1-(2-(2,4-dioxotetrahydropyrimidin-1(2H)-yl)-1,3-dioxoisoindolin-5-yl)piperidin-4-yl)piperazin-1-yl)methyl)cyclohexyl)-6-methoxy-2H-indazol-5-yl)-3-(trifluoromethyl)benzamide (GT-08671)

With reference to the method of Scheme T-3, the target compound (GT-08671) was obtained (yellow solid, 38 mg, yield 49.44%). ¹H NMR (400 MHz, DMSO) δ 12.20 (s, 1H), 11.41 (s, 1H), 10.85 (s, 1H), 9.78 (s, 1H), 8.29 (dd, J=12.3, 5.6 Hz, 3H), 8.02 (s, 1H), 7.97 (d, J=7.7 Hz, 1H), 7.78 (dd, J=14.1, 8.2 Hz, 2H), 7.48 (s, 1H), 7.37 (d, J=8.6 Hz, 1H), 7.07 (s, 1H), 4.42 (t, J=11.7 Hz, 1H), 4.30 (d, J=13.1 Hz, 2H), 3.87 (s, 3H), 3.80 (d, J=6.8 Hz, 2H), 3.72 (s, 3H), 3.48 (s, 4H), 3.02 (t, J=12.4 Hz, 3H), 2.82 (dd, J=15.8, 8.9 Hz, 2H), 2.27-2.05 (m, 6H), 1.94 (dd, J=22.5, 11.7 Hz, 3H), 1.76 (d, J=10.9 Hz, 2H), 1.25 (d, J=10.5 Hz, 2H). LCMS (ESI) calcd, for C₄₄H₄₉F₃N₉O₆⁺ [M+H]⁺: 856.38, found, 856.4.

### Example 151: Preparation of N-(2-((1r,4r)-4-((4-(1-(2-(2,4-dioxotetrahydropyrimidin-1(2H)-yl)-1,3-dioxoisoindolin-5-yl)-3,3-difluoropiperidin-4-yl)piperazin-1-yl)methyl)cyclohexyl)-6-methoxy-2H-indazol-5-yl)-3-(trifluoromethyl)benzamide (GT-08672)

With reference to the method of Scheme T-3, the target compound (GT-08672) was obtained (yellow solid, 6 mg, yield 7.49%). ¹H NMR (400 MHz, DMSO) § 10.85 (s, 1H), 9.77 (s, 1H), 9.23 (s, 1H), 8.35-8.24 (m, 4H), 8.01 (s, 1H), 7.97 (d, J=8.1 Hz, 1H), 7.77 (dd, J=16.9, 8.3 Hz, 2H), 7.52 (s, 1H), 7.41 (d, J=8.8 Hz, 1H), 7.06 (s, 1H), 4.45 (d, J=30.5 Hz, 2H), 4.26 (d, J=12.7 Hz, 1H), 3.87 (s, 3H), 3.79 (t, J=6.8 Hz, 2H), 3.59 (s, 4H), 3.45-3.39 (m, 3H), 3.02 (d, J=28.3 Hz, 8H), 2.83 (t, J=7.0 Hz, 2H), 2.16 (s, 2H), 1.94 (dd, J=26.0, 14.4 Hz, 8H). LCMS (ESI) calcd, for C₄₄H₄₇F₅N₉O₆⁺ [M+H]⁺: 892.36, found, 892.4.

### Example 152: Preparation of N-(2-((1r,4r)-4-((5-(2-(2,4-dioxotetrahydropyrimidin-1(2H)-yl)-1,3-dioxoisoindolin-5-yl)-2,5-diazabicyclo[2.2.2]octan-2-yl)methyl)cyclohexyl)-6-methoxy-2H-indazol-5-yl)-3-(trifluoromethyl)benzamide (GT-08673)

With reference to the method of Scheme T-3, the target compound (GT-08673) was obtained (yellow solid, 43 mg, yield 59.95%). ¹H NMR (400 MHz, DMSO) δ 10.85 (s, 1H), 10.45 (s, 1H), 9.78 (s, 1H), 8.29 (dd, J=13.8, 7.3 Hz, 3H), 8.02 (s, 1H), 7.97 (d, J=7.8 Hz, 1H), 7.79 (t, J=7.6 Hz, 2H), 7.26 (s, 1H), 7.07 (s, 2H), 4.61 (s, 1H), 4.42 (d, J=3.4 Hz, 1H), 4.24-4.05 (m, 1H), 4.00 (d, J=8.8 Hz, 1H), 3.87 (s, 4H), 3.79 (t, J=6.7 Hz, 2H), 3.58 (dd, J=33.5, 17.2 Hz, 2H), 3.35-3.18 (m, 3H), 2.84 (t, J=6.7 Hz, 2H), 2.19-1.85 (m, 10H), 1.27 (d, J=12.6 Hz, 2H). LCMS (ESI) calcd, for C₄₁H₄₂F₃N₈O₆⁺ [M+H]⁺: 799.32, found, 799.3.

### Example 153: Preparation of 1-(5-((4-(4-((9-cyclopentyl-8-(phenylamino)-9H-purin-2-yl)amino)phenyl)piperazin-1-yl)methyl)-1-oxoisoindolin-2-yl)dihydropyrimidine-2,4 (1H,3H)-dione (GT-05836)

With reference to the method of Step 2 in Scheme T-1, the target compound (GT-05836) was obtained (yellow solid, 10 mg, yield 15.96%). ¹H NMR (400 MHz, DMSO-d₆) δ 11.34 (s, 1H), 10.70 (s, 1H), 10.08 (s, 2H), 8.40 (s, 1H), 7.97-7.87 (m, 2H), 7.87-7.76 (m, 3H), 7.48 (d, J=7.8 Hz, 2H), 7.40 (t, J=7.3 Hz, 2H), 7.13 (t, J=7.2 Hz, 1H), 7.02 (d, J=7.9 Hz, 2H), 5.12 (s, 1H), 4.83 (d, J=16.2 Hz, 1H), 4.58 (d, J=29.3 Hz, 3H), 3.79 (d, J=5.4 Hz, 4H), 3.19 (s, 5H), 2.82 (d, J=29.4 Hz, 3H), 2.30 (s, 2H), 2.07 (s, 2H), 1.90 (s, 2H), 1.62 (s, 2H). LCMS (ESI) calcd, for C₃₉H₄₂N₁₁O₃⁺ [M+H]⁺: 712.35, found, 712.4.

### Example 154: Preparation of 1-(5-(((1-(4-((9-cyclopentyl-8-(phenylamino)-9H-purin-2-yl)amino)phenyl)piperidin-4-yl)(methyl)amino)methyl)-1-oxoisoindolin-2-yl)dihydropyrimidine-2,4 (1H,3H)-dione (GT-05842)

With reference to the method of Step 2 in Scheme T-1, the target compound (GT-05842) was obtained (yellow solid, 13 mg, yield 21.20%). ¹H NMR (400 MHz, DMSO-d₆) δ 11.39 (s, 1H), 10.70 (s, 1H), 10.20 (d, J=33.3 Hz, 2H), 8.45 (s, 1H), 8.01 (s, 1H), 7.88 (s, 2H), 7.80 (d, J=7.9 Hz, 2H), 7.66 (s, 2H), 7.43 (dd, J=20.7, 13.4 Hz, 4H), 7.15 (t, J=7.4 Hz, 1H), 5.29-5.11 (m, 1H), 4.83 (d, J=15.9 Hz, 1H), 4.61 (s, 2H), 4.46 (s, 1H), 3.79 (d, J=6.5 Hz, 6H), 3.31-3.10 (m, 4H), 2.94-2.73 (m, 3H), 2.67 (s, 3H), 2.36 (s, 4H), 2.11 (s, 2H), 1.95 (s, 2H), 1.66 (s, 2H). LCMS (ESI) calcd, for C₄₁H₄₆N₁₁O₃⁺ [M+H]⁺: 740.38, found, 740.4.

### Example 155: Preparation of 5-(((1-(4-((9-cyclopentyl-8-(phenylamino)-9H-purin-2-yl)amino)phenyl)piperidin-4-yl)(methyl)amino)methyl)-2-(2,4-dioxotetrahydropyrimidin-1(2H)-yl)isoindoline-1,3-dione (GT-07113)

With reference to the method of Step 2 in Scheme T-1, the target compound (GT-07113) was obtained (yellow solid, 24 mg, yield 38.41%). ¹H NMR (400 MHz, DMSO-d₆) δ 11.70 (s, 1H), 10.93 (s, 1H), 10.39 (s, 2H), 8.47 (s, 2H), 8.33 (d, J=9.8 Hz, 1H), 8.14-8.06 (m, 1H), 7.79 (d, J=7.8 Hz, 2H), 7.71 (d, J=8.6 Hz, 2H), 7.63 (s, 2H), 7.42 (t, J=7.9 Hz, 2H), 7.18 (d, J=7.4 Hz, 1H), 5.25 (s, 1H), 4.76-4.39 (m, 3H), 3.93 (s, 4H), 3.45 (s, 3H), 2.86 (t, J=6.8 Hz, 2H), 2.65 (d, J=10.5 Hz, 3H), 2.38 (dd, J=28.5, 20.6 Hz, 5H), 2.12 (s, 2H), 1.94 (s, 2H), 1.73-1.60 (m, 2H). LCMS (ESI) calcd, for C₄₁H₄₄N₁₁O₄⁺[M+H]⁺: 754.36, found, 754.3.

### Example 156: Preparation of 5-(4-(((1-(4-((9-cyclopentyl-8-(phenylamino)-9H-purin-2-yl)amino)phenyl)piperidin-4-yl)(methyl)amino)methyl)piperidin-1-yl)-2-(2,4-dioxotetrahydropyrimidin-1(2H)-yl)isoindoline-1,3-dione (GT-05843)

With reference to the method of Scheme T-3, the target compound (GT-05843) was obtained (yellow solid, 11 mg, yield 15.86%). ¹H NMR (400 MHz, DMSO-d₆) δ 10.84 (s, 1H), 10.45-9.94 (m, 3H), 8.44 (s, 1H), 7.77 (dd, J=27.7, 8.7 Hz, 3H), 7.63 (s, 2H), 7.37 (dd, J=28.9, 9.2 Hz, 5H), 7.14 (t, J=7.2 Hz, 1H), 5.25-5.11 (m, 1H), 4.15 (d, J=13.0 Hz, 2H), 3.78 (s, 6H), 3.16 (s, 2H), 2.99 (d, J=33.1 Hz, 3H), 2.81 (d, J=17.4 Hz, 4H), 2.33 (s, 3H), 2.16 (d, J=45.6 Hz, 7H), 1.94 (s, 3H), 1.65 (s, 2H), 1.27 (d, J=20.1 Hz, 3H). LCMS (ESI) calcd. for C₄₆H₅₃N₁₂O₄⁺ [M+H]⁺: 837.43, found, 837.5.

### Example 157: Preparation of 4-(4-(((1-(4-((9-cyclopentyl-8-(phenylamino)-9H-purin-2-yl)amino)phenyl)piperidin-4-yl)(methyl)amino)methyl)piperidin-1-yl)-2-(2,4-dioxotetrahydropyrimidin-1(2H)-yl)isoindoline-1,3-dione (GT-05844)

With reference to the method of Scheme T-3, the target compound (GT-05844) was obtained (yellow solid, 15 mg, yield 21.62%). ¹H NMR (400 MHz, DMSO-d₆) δ 10.86 (s, 1H), 10.07 (s, 3H), 8.43 (s, 1H), 7.77 (dd, J=22.4, 7.8 Hz, 3H), 7.62 (s, 2H), 7.40 (d, J=7.2 Hz, 5H), 7.12 (dd, J=18.4, 10.6 Hz, 1H), 5.22-5.03 (m, 1H), 3.78 (d, J=5.7 Hz, 8H), 3.21-3.14 (m, 4H), 2.99 (s, 3H), 2.78 (d, J=17.3 Hz, 6H), 2.33 (s, 3H), 2.09 (s, 6H), 1.95 (s, 3H), 1.66 (s, 2H). LCMS (ESI) calcd, for C₄₆H₅₃N₁₂O₄⁺ [M+H]⁺: 837.43, found, 837.5.

### Example 158: Preparation of 1-(5-((4-(1-(4-((9-cyclopentyl-8-(phenylamino)-9H-purin-2-yl)amino)phenyl)piperidin-4-yl)piperazin-1-yl)methyl)-1-oxoisoindolin-2-yl)dihydropyrimidine-2,4 (1H,3H)-dione (GT-05914)

With reference to the method of Step 2 in Scheme T-1, the target compound (GT-05914) was obtained (yellow solid, 37 mg, yield 62.56%). ¹H NMR (400 MHz, DMSO-d₆) δ 10.69 (s, 1H), 10.15 (s, 2H), 8.43 (s, 1H), 7.99-7.77 (m, 5H), 7.61 (s, 2H), 7.41 (t, J=7.6 Hz, 4H), 7.14 (s, 1H), 5.27-5.06 (m, 1H), 4.81 (d, J=16.3 Hz, 1H), 4.66-4.46 (m, 3H), 3.80 (s, 8H), 3.13 (s, 5H), 2.82 (d, J=29.6 Hz, 3H), 2.54 (s, 1H), 2.37-2.22 (m, 4H), 2.10 (s, 4H), 1.94 (s, 2H), 1.66 (s, 2H). LCMS (ESI) calcd, for C₄₄H₅₁N₁₂O₃⁺ [M+H]⁺: 795.42, found, 795.5.

### Example 159: Preparation of 5-((4-(1-(4-((9-cyclopentyl-8-(phenylamino)-9H-purin-2-yl)amino)phenyl)piperidin-4-yl)piperazin-1-yl)methyl)-2-(2,4-dioxotetrahydropyrimidin-1(2H)-yl)isoindoline-1,3-dione (GT-06124)

With reference to the method of Step 2 in Scheme T-1, the target compound (GT-06124) was obtained (yellow solid, 16 mg, yield 21.27%). ¹H NMR (400 MHz, DMSO-d₆) δ 10.93 (s, 1H), 10.70-10.43 (m, 1H), 10.19 (s, 2H), 8.44 (s, 1H), 8.34-8.07 (m, 2H), 7.81 (d, J=8.1 Hz, 2H), 7.61 (s, 2H), 7.41 (t, J=7.8 Hz, 4H), 7.14 (t, J=7.3 Hz, 1H), 5.23-5.02 (m, 1H), 4.49 (s, 2H), 3.82 (dd, J=14.8, 8.2 Hz, 6H), 3.41-3.31 (m, 4H), 3.12 (s, 4H), 2.86 (t, J=6.7 Hz, 2H), 2.76 (t, J=6.6 Hz, 1H), 2.32 (d, J=6.8 Hz, 4H), 2.10 (s, 4H), 1.94 (s, 2H), 1.65 (d, J=5.5 Hz, 2H). LCMS (ESI) calcd, for C₄₄H₄₉N₁₂O₄⁺ [M+H]⁺: 809.40, found, 809.4.

### Example 160: Preparation of 5-(4-((4-(1-(4-((9-cyclopentyl-8-(phenylamino)-9H-purin-2-yl)amino)phenyl)piperidin-4-yl)piperazin-1-yl)methyl)piperidin-1-yl)-2-(2,4-dioxotetrahydropyrimidin-1(2H)-yl)isoindoline-1,3-dione (GT-05955)

With reference to the method of Scheme T-3, the target compound (GT-05955) was obtained (yellow solid, 20 mg, yield 30.14%). ¹H NMR (400 MHz, DMSO-d₆) δ 10.89 (d, J=36.1 Hz, 1H), 10.34 (dd, J=64.3, 28.8 Hz, 3H), 8.44 (s, 1H), 7.79 (d, J=7.9 Hz, 2H), 7.69-7.59 (m, 2H), 7.42 (t, J=7.8 Hz, 5H), 7.16 (t, J=7.5 Hz, 1H), 5.25-5.08 (m, 1H), 4.12 (d, J=13.1 Hz, 2H), 3.84-3.77 (m, 8H), 3.04 (d, J=12.2 Hz, 4H), 2.91-2.70 (m, 5H), 2.34 (s, 4H), 2.02 (dd, J=38.7, 26.5 Hz, 11H), 1.66 (s, 2H), 1.27 (s, 3H). LCMS (ESI) calcd. for C₄₉H₅₈N₁₃O₄⁺ [M+H]⁺: 892.47, found, 892.5.

### Example 161: Preparation of 4-(4-((4-(1-(4-((9-cyclopentyl-8-(phenylamino)-9H-purin-2-yl)amino)phenyl)piperidin-4-yl)piperazin-1-yl)methyl)piperidin-1-yl)-2-(2,4-dioxotetrahydropyrimidin-1(2H)-yl)isoindoline-1,3-dione (GT-05956)

With reference to the method of Scheme T-3, the target compound (GT-05956) was obtained (yellow solid, 14 mg, yield 21.10%). ¹H NMR (400 MHz, DMSO-d₆) δ 10.89 (d, J=32.4 Hz, 1H), 10.22 (s, 2H), 8.44 (s, 1H), 7.77 (dd, J=21.6, 7.2 Hz, 3H), 7.63 (s, 2H), 7.51-7.36 (m, 7H), 7.25-7.09 (m, 2H), 5.17 (d, J=8.1 Hz, 1H), 3.81 (s, 8H), 3.37-3.31 (m, 4H), 3.18 (s, 4H), 2.85 (dd, J=34.4, 16.2 Hz, 5H), 2.33 (s, 4H), 2.10 (s, 7H), 1.93 (d, J=15.1 Hz, 4H), 1.66 (s, 2H), 1.40 (d, J=50.0 Hz, 2H). LCMS (ESI) calcd, for C₄₉H₅₈N₁₃O₄⁺ [M+H]⁺: 892.47, found, 892.5.

### Example 162: Preparation of 1-(5-((4-(4-((5-bromo-4-((5-(dimethylphosphoryl)quinoxalin-6-yl)amino)pyrimidin-2-yl)amino)-5-methoxy-2-methylphenyl)piperazin-1-yl)methyl)-1-oxoisoindolin-2-yl)dihydropyrimidine-2,4 (1H,3H)-dione (GT-05946)

With reference to the method of Step 2 in Scheme T-1, the target compound (GT-05946) was obtained (yellow solid, 22 mg, yield 38.45%). ¹H NMR (400 MHz, DMSO-d₆) δ 12.87 (s, 1H), 11.01 (s, 1H), 10.70 (s, 1H), 8.89 (d, J=10.4 Hz, 3H), 8.59 (s, 1H), 8.33 (s, 1H), 7.90 (dt, J=25.5, 11.8 Hz, 4H), 7.46 (s, 1H), 6.74 (s, 1H), 4.72 (dd, J=93.5, 18.1 Hz, 4H), 3.81 (s, 5H), 3.20 (s, 8H), 2.83 (d, J=29.3 Hz, 2H), 2.16-1.98 (m, 9H). LCMS (ESI) calcd, for C₃₉H₄₂BrN₁₁O₅P⁺ [M+H]⁺: 854.23, found, 854.3.

### Example 163: Preparation of 1-(5-(((1-(4-((5-bromo-4-((5-(dimethylphosphoryl)quinoxalin-6-yl)amino)pyrimidin-2-yl)amino)-5-methoxy-2-methylphenyl)piperidin-4-yl)(methyl)amino)methyl)-1-oxoisoindolin-2-yl)dihydropyrimidine-2,4 (1H,3H)-dione (GT-05948)

With reference to the method of Step 2 in Scheme T-1, the target compound (GT-05948) was obtained (yellow solid, 30 mg, yield 42.52%). ¹H NMR (400 MHz, DMSO-d₆) δ 11.22 (s, 1H), 10.70 (s, 1H), 10.10 (s, 1H), 9.34 (s, 2H), 8.51 (s, 1H), 8.35 (s, 1H), 8.22 (d, J=9.2 Hz, 1H), 8.13 (d, J=9.0 Hz, 1H), 8.01 (d, J=6.7 Hz, 1H), 7.88 (s, 2H), 7.36 (s, 1H), 6.78 (s, 1H), 4.83 (d, J=11.2 Hz, 1H), 4.62 (s, 2H), 4.42 (s, 1H), 3.81 (s, 6H), 3.34 (s, 1H), 3.13 (s, 2H), 2.82 (d, J=31.4 Hz, 4H), 2.67 (s, 3H), 2.26 (d, J=22.6 Hz, 2H), 2.03 (s, 2H), 1.85 (d, J=13.6 Hz, 6H), 1.70 (s, 2H). LCMS (ESI) calcd, for C₄₁H₄₆BrN₁₁O₅P⁺ [M+H]⁺: 882.26, found, 882.3.

### Example 164: Preparation of 5-(((1-(4-((5-bromo-4-((5-(dimethylphosphoryl)quinoxalin-6-yl)amino)pyrimidin-2-yl)amino)-5-methoxy-2-methylphenyl)piperidin-4-yl)(methyl)amino)methyl)-2-(2,4-dioxotetrahydropyrimidin-1(2H)-yl)isoindoline-1,3-dione (GT-06125)

With reference to the method of Step 2 in Scheme T-1, the target compound (GT-06125) was obtained (yellow solid, 50 mg, yield 69.75%). ¹H NMR (400 MHz, DMSO-d₆) δ 10.94 (s, 2H), 9.79 (s, 1H), 9.30 (s, 1H), 8.86 (s, 1H), 8.42-8.34 (m, 2H), 8.24 (d, J=8.7 Hz, 2H), 8.10 (dd, J=8.3, 5.5 Hz, 2H), 7.39 (s, 1H), 6.73 (s, 1H), 4.82-4.60 (m, 1H), 4.42 (d, J=58.6 Hz, 1H), 3.82 (d, J=9.0 Hz, 4H), 3.65 (d, J=6.9 Hz, 2H), 3.10 (s, 3H), 2.86 (t, J=6.8 Hz, 2H), 2.75-2.63 (m, 5H), 2.23 (s, 2H), 1.94 (d, J=29.6 Hz, 2H), 1.84 (d, J=13.7 Hz, 6H), 1.73 (s, 2H). LCMS (ESI) calcd, for C₄₁H₄₄BrN₁₁O₆P⁺ [M+H]⁺: 896.24, found, 896.3.

### Example 165: Preparation of 5-(4-(((1-(4-((5-bromo-4-((5-(dimethylphosphoryl)quinoxalin-6-yl)amino)pyrimidin-2-yl)amino)-5-methoxy-2-methylphenyl)piperidin-4-yl)(methyl)amino)methyl)piperidin-1-yl)-2-(2,4-dioxotetrahydropyrimidin-1(2H)-yl)isoindoline-1,3-dione (GT-05949)

With reference to the method of Scheme T-3, the target compound (GT-05949) was obtained (yellow solid, 20 mg, yield 25.53%). ¹H NMR (400 MHz, DMSO-d₆) δ 10.86 (s, 1H), 9.84 (s, 2H), 9.30 (s, 1H), 8.94 (s, 1H), 8.44 (s, 1H), 8.35 (s, 1H), 8.24 (d, J=8.8 Hz, 1H), 8.10 (d, J=8.8 Hz, 1H), 7.74 (d, J=8.8 Hz, 1H), 7.37 (dd, J=30.2, 11.0 Hz, 3H), 6.74 (s, 1H), 4.16 (d, J=12.7 Hz, 2H), 3.80 (d, J=11.0 Hz, 4H), 3.37-3.30 (m, 3H), 3.05 (d, J=12.9 Hz, 4H), 2.96-2.73 (m, 8H), 2.13 (d, J=24.1 Hz, 4H), 1.84 (d, J=13.4 Hz, 9H), 1.72 (s, 2H), 1.27 (d, J=23.6 Hz, 2H). LCMS (ESI) calcd, for C₄₆H₅₃BrN₁₂O₆P⁺ [M+H]⁺: 979.31, found, 979.3.

### Example 166: Preparation of 4-(4-(((1-(4-((5-bromo-4-((5-(dimethylphosphoryl)quinoxalin-6-yl)amino)pyrimidin-2-yl)amino)-5-methoxy-2-methylphenyl)piperidin-4-yl)(methyl)amino)methyl)piperidin-1-yl)-2-(2,4-dioxotetrahydropyrimidin-1(2H)-yl)isoindoline-1,3-dione (GT-05950)

With reference to the method of Scheme T-3, the target compound (GT-05950) was obtained (yellow solid, 23 mg, yield 29.36%). ¹H NMR (400 MHz, DMSO-d₆) δ 10.87 (s, 1H), 10.08 (d, J=53.6 Hz, 2H), 9.33 (s, 1H), 9.20 (s, 1H), 8.49 (s, 1H), 8.35 (s, 1H), 8.23 (d, J=9.2 Hz, 1H), 8.13 (d, J=9.1 Hz, 1H), 7.76 (s, 1H), 7.40 (dd, J=16.8, 8.6 Hz, 3H), 6.78 (s, 1H), 3.80 (dd, J=13.8, 6.7 Hz, 9H), 3.39-3.34 (m, 2H), 3.12 (s, 3H), 2.98 (d, J=11.4 Hz, 3H), 2.83 (d, J=11.7 Hz, 5H), 2.20 (s, 4H), 1.85 (d, J=13.6 Hz, 9H), 1.71 (s, 2H), 1.48 (s, 2H). LCMS (ESI) calcd, for C₄₆H₅₃BrN₁₂O₆P⁺ [M+H]⁺: 979.31, found, 979.3.

### Example 167: Preparation of 1-(5-((4-(1-(4-((5-bromo-4-((5-(dimethylphosphoryl)quinoxalin-6-yl)amino)pyrimidin-2-yl)amino)-5-methoxy-2-methylphenyl)piperidin-4-yl)piperazin-1-yl)methyl)-1-oxoisoindolin-2-yl)dihydropyrimidine-2,4 (1H,3H)-dione (GT-05996)

With reference to the method of Step 2 in Scheme T-1, the target compound (GT-05996) was obtained (yellow solid, 22 mg, yield 31.93%). ¹H NMR (400 MHz, DMSO-d₆) δ 10.69 (s, 1H), 9.77 (s, 1H), 9.30 (s, 1H), 8.88 (s, 1H), 8.42 (s, 1H), 8.37 (s, 1H), 8.24 (d, J=9.0 Hz, 1H), 8.10 (d, J=9.0 Hz, 1H), 7.87 (d, J=13.0 Hz, 2H), 7.79 (d, J=7.5 Hz, 1H), 7.40 (s, 1H), 6.73 (s, 1H), 4.95-4.79 (m, 1H), 4.57 (d, J=17.0 Hz, 1H), 4.42 (s, 2H), 3.81 (s, 5H), 3.69 (s, 2H), 3.55 (s, 7H), 3.11 (s, 2H), 2.91-2.66 (m, 5H), 2.15 (s, 2H), 1.84 (d, J=13.7 Hz, 8H), 1.73 (s, 2H). LCMS (ESI) calcd, for C₄₄H₅₁BrN₁₂O₅P⁺ [M+H]⁺: 937.30, found, 937.3.

### Example 168: Preparation of 5-((4-(1-(4-((5-bromo-4-((5-(dimethylphosphoryl)quinoxalin-6-yl)amino)pyrimidin-2-yl)amino)-5-methoxy-2-methylphenyl)piperidin-4-yl)piperazin-1-yl)methyl)-2-(2,4-dioxotetrahydropyrimidin-1(2H)-yl)isoindoline-1,3-dione (GT-06154)

With reference to the method of Step 2 in Scheme T-1, the target compound (GT-06154) was obtained (yellow solid, 42 mg, yield 60.06%). ¹H NMR (400 MHz, DMSO-d₆) δ 10.93 (s, 1H), 9.92 (s, 1H), 9.30 (s, 1H), 9.07 (s, 1H), 8.44 (s, 1H), 8.35 (s, 1H), 8.24 (d, J=28.3 Hz, 1H), 8.09 (dd, J=10.5, 8.5 Hz, 2H), 7.96-7.83 (m, 1H), 7.37 (s, 1H), 6.73 (s, 1H), 4.45 (s, 2H), 3.81 (d, J=7.2 Hz, 4H), 3.65 (s, 10H), 3.12 (d, J=9.4 Hz, 2H), 2.90-2.65 (m, 5H), 2.16 (s, 2H), 1.86 (t, J=16.6 Hz, 8H), 1.70 (s, 2H). LCMS (ESI) calcd, for C₄₄H₄₉BrN₁₂O₆P⁺ [M+H]⁺: 951.28, found, 951.3.

### Example 169: Preparation of 5-(4-((4-(1-(4-((5-bromo-4-((5-(dimethylphosphoryl)quinoxalin-6-yl)amino)pyrimidin-2-yl)amino)-5-methoxy-2-methylphenyl)piperidin-4-yl)piperazin-1-yl)methyl)piperidin-1-yl)-2-(2,4-dioxotetrahydropyrimidin-1(2H)-yl)isoindoline-1,3-dione (GT-05997)

With reference to the method of Scheme T-3, the target compound (GT-05997) was obtained (yellow solid, 36 mg, yield 47.35%). ¹H NMR (400 MHz, DMSO-d₆) δ 10.85 (s, 1H), 9.83 (s, 1H), 9.31 (s, 1H), 8.95 (s, 1H), 8.43 (s, 1H), 8.37 (s, 1H), 8.24 (d, J=9.4 Hz, 1H), 8.11 (d, J=9.1 Hz, 1H), 7.72 (t, J=7.0 Hz, 1H), 7.41 (d, J=9.9 Hz, 2H), 7.32 (d, J=7.8 Hz, 1H), 6.74 (s, 1H), 4.13 (d, J=12.4 Hz, 2H), 3.86-3.72 (m, 12H), 3.21-2.97 (m, 8H), 2.83 (t, J=7.0 Hz, 2H), 2.73 (s, 2H), 2.19 (s, 3H), 2.08-1.90 (m, 4H), 1.85 (d, J=13.7 Hz, 7H), 1.74 (s, 2H), 1.28 (d, J=11.7 Hz, 2H). LCMS (ESI) calcd, for C₄₉H₅₈BrN₁₃O₆P⁺ [M+H]⁺: 1034.36, found, 1034.4.

### Example 170: Preparation of 4-(4-((4-(1-(4-((5-bromo-4-((5-(dimethylphosphoryl)quinoxalin-6-yl)amino)pyrimidin-2-yl)amino)-5-methoxy-2-methylphenyl)piperidin-4-yl)piperazin-1-yl)methyl)piperidin-1-yl)-2-(2,4-dioxotetrahydropyrimidin-1(2H)-yl)isoindoline-1,3-dione (GT-05998)

With reference to the method of Scheme T-3, the target compound (GT-05998) was obtained (yellow solid, 30 mg, yield 39.46%). ¹H NMR (400 MHz, DMSO-d₆) δ 11.90 (d, J=261.8 Hz, 1H), 10.86 (s, 1H), 9.96 (s, 1H), 9.33 (s, 1H), 9.14 (s, 1H), 8.47 (s, 1H), 8.37 (s, 1H), 8.22 (s, 1H), 8.12 (d, J=8.8 Hz, 1H), 7.74 (d, J=6.8 Hz, 1H), 7.46-7.33 (m, 4H), 6.76 (s, 1H), 3.81 (d, J=13.9 Hz, 14H), 3.16 (s, 4H), 2.95-2.70 (m, 7H), 2.20 (s, 2H), 2.08 (s, 3H), 1.88 (t, J=20.3 Hz, 10H), 1.73 (s, 3H), 1.47 (s, 2H). LCMS (ESI) calcd. for C₄₉H₅₈BrN₁₃O₆P⁺ [M+H]⁺: 1034.36, found, 1034.4.

### Example 171: Preparation of 1-(5-((4-(6-((2-(1-(cyclopropylsulfonyl)-1H-pyrazol-4-yl)pyrimidin-4-yl)amino)-1-isopropyl-1H-pyrazolo[4,3-c]pyridin-3-yl)piperazin-1-yl)methyl)-1-oxoisoindolin-2-yl)dihydropyrimidine-2,4 (1H,3H)-dione (GT-06010)

With reference to the method of Step 2 in Scheme T-1, the target compound (GT-06010) was obtained (yellow solid, 35 mg, yield 58.11%). ¹H NMR (400 MHz, DMSO-d₆) δ 11.67 (s, 1H), 11.38 (s, 1H), 10.70 (s, 1H), 9.12 (d, J=7.9 Hz, 1H), 9.00 (s, 1H), 8.63 (d, J=5.6 Hz, 1H), 8.48 (dd, J=34.5, 6.1 Hz, 1H), 8.20 (s, 1H), 7.97 (s, 1H), 7.88 (dd, J=16.8, 8.0 Hz, 2H), 7.27 (s, 1H), 4.98-4.76 (m, 2H), 4.60 (d, J=13.3 Hz, 3H), 4.12 (d, J=12.1 Hz, 2H), 3.80 (s, 4H), 3.49-3.42 (m, 4H), 3.31 (s, 2H), 2.93-2.73 (m, 2H), 1.48 (d, J=6.2 Hz, 6H), 1.37 (s, 1H), 1.29 (d, J=7.1 Hz, 1H), 0.66-0.50 (m, 1H). LCMS (ESI) calcd, for C₃₆H₄₀N₁₃O₅S⁺ [M+H]⁺: 766.30, found, 766.3.

### Example 172: Preparation of 5-(4-((4-(6-((2-(1-(cyclopropylsulfonyl)-1H-pyrazol-4-yl)pyrimidin-4-yl)amino)-1-isopropyl-1H-pyrazolo[4,3-c]pyridin-3-yl)piperazin-1-yl)methyl)piperidin-1-yl)-2-(2,4-dioxotetrahydropyrimidin-1(2H)-yl)isoindoline-1,3-dione (GT-06073)

With reference to the method of Scheme T-3, the target compound (GT-06073) was obtained (yellow solid, 26 mg, yield 38.31%). ¹H NMR (400 MHz, DMSO-d₆) δ 11.82 (s, 1H), 10.85 (s, 1H), 10.72 (s, 1H), 9.20 (d, J=18.8 Hz, 1H), 8.71-8.56 (m, 2H), 8.55-8.40 (m, 1H), 8.15 (d, J=6.1 Hz, 1H), 7.80-7.63 (m, 1H), 7.44 (dd, J=13.5, 5.3 Hz, 1H), 7.33 (dt, J=30.2, 20.1 Hz, 1H), 7.02 (dd, J=69.6, 37.2 Hz, 1H), 4.88-4.78 (m, 1H), 4.15 (s, 2H), 3.90 (s, 8H), 3.44 (d, J=7.0 Hz, 2H), 3.30 (dd, J=7.9, 4.7 Hz, 4H), 3.12 (dd, J=30.0, 12.6 Hz, 4H), 3.00-2.92 (m, 1H), 2.86-2.71 (m, 2H), 2.25 (s, 1H), 2.01-1.91 (m, 2H), 1.50 (d, J=6.6 Hz, 6H), 1.41-1.27 (m, 2H). LCMS (ESI) calcd, for C₄₁H₄₇N₁₄O₆S⁺ [M+H]⁺: 863.35, found, 863.4.

### Example 173: Preparation of 1-(5-(((1-(6-((2-(1-(cyclopropylsulfonyl)-1H-pyrazol-4-yl)pyrimidin-4-yl)amino)-1-isopropyl-1H-pyrazolo[4,3-c]pyridin-3-yl)piperidin-4-yl)(methyl)amino)methyl)-1-oxoisoindolin-2-yl)dihydropyrimidine-2,4 (1H,3H)-dione (GT-06019)

With reference to the method of Step 2 in Scheme T-1, the target compound (GT-06019) was obtained (yellow solid, 35 mg, yield 59.15%). ¹H NMR (400 MHz, DMSO-d₆) δ 11.93 (s, 1H), 10.89 (s, 1H), 10.69 (s, 1H), 9.19 (s, 1H), 8.60 (s, 2H), 8.45 (d, J=6.9 Hz, 1H), 8.01 (s, 1H), 7.96 (s, 1H), 7.86 (s, 2H), 7.30 (d, J=50.8 Hz, 1H), 4.90-4.77 (m, 2H), 4.70-4.54 (m, 2H), 4.38 (dd, J=12.5, 6.6 Hz, 1H), 4.21 (d, J=11.9 Hz, 2H), 3.95 (s, 2H), 3.55-3.48 (m, 4H), 3.02 (d, J=11.4 Hz, 3H), 2.89-2.76 (m, 2H), 2.64 (d, J=3.7 Hz, 3H), 2.32 (dd, J=24.3, 11.2 Hz, 2H), 2.08-1.92 (m, 3H), 1.47 (d, J=6.5 Hz, 6H). LCMS (ESI) calcd, for C₃₈H₄₄N₁₃O₅S⁺ [M+H]⁺: 794.33, found, 794.4.

### Example 174: Preparation of 1-(5-((4-(1-(6-((2-(1-(cyclopropylsulfonyl)-1H-pyrazol-4-yl)pyrimidin-4-yl)amino)-1-isopropyl-1H-pyrazolo[4,3-c]pyridin-3-yl)piperidin-4-yl)piperazin-1-yl)methyl)-1-oxoisoindolin-2-yl)dihydropyrimidine-2,4 (1H,3H)-dione (GT-06077)

With reference to the method of Step 2 in Scheme T-1, the target compound (GT-06077) was obtained (yellow solid, 25 mg, yield 34.85%). ¹H NMR (400 MHz, DMSO-d₆) δ 12.03 (s, 2H), 10.70 (s, 1H), 9.23 (s, 1H), 8.64 (s, 2H), 8.46 (d, J=7.0 Hz, 1H), 7.97 (s, 1H), 7.87 (s, 2H), 7.28 (d, J=16.3 Hz, 1H), 4.83 (d, J=5.5 Hz, 2H), 4.61 (d, J=14.4 Hz, 4H), 4.21 (d, J=11.7 Hz, 4H), 3.81-3.53 (m, 12H), 3.01 (t, J=12.1 Hz, 2H), 2.94-2.76 (m, 2H), 2.24 (d, J=9.4 Hz, 2H), 1.97 (ddd, J=20.8, 11.8, 5.1 Hz, 3H), 1.48 (d, J=6.5 Hz, 6H). LCMS (ESI) calcd, for C₄₁H₄₉N₁₄O₅S⁺ [M+H]⁺: 849.37, found, 849.4.

### Example 175: Preparation of 5-((4-(1-(6-((2-(1-(cyclopropylsulfonyl)-1H-pyrazol-4-yl)pyrimidin-4-yl)amino)-1-isopropyl-1H-pyrazolo[4,3-c]pyridin-3-yl)piperidin-4-yl)piperazin-1-yl)methyl)-2-(2,4-dioxotetrahydropyrimidin-1(2H)-yl)isoindoline-1,3-dione (GT-06126)

With reference to the method of Step 2 in Scheme T-1, the target compound (GT-06126) was obtained (yellow solid, 32 mg, yield 43.88%). ¹H NMR (400 MHz, DMSO-d₆) δ 11.27 (s, 1H), 10.92 (d, J=6.4 Hz, 1H), 9.15 (s, 1H), 8.92 (s, 1H), 8.61 (s, 1H), 8.52 (d, J=6.2 Hz, 1H), 8.26 (s, 1H), 8.18-7.93 (m, 3H), 7.22 (s, 1H), 4.80 (dt, J=13.1, 6.6 Hz, 1H), 4.41 (s, 2H), 4.18 (d, J=12.0 Hz, 2H), 3.84 (d, J=6.7 Hz, 2H), 3.66 (s, 4H), 3.31-3.25 (m, 4H), 3.00 (t, J=11.9 Hz, 3H), 2.86 (t, J=6.8 Hz, 2H), 2.76 (t, J=6.6 Hz, 1H), 2.23 (d, J=8.9 Hz, 2H), 1.90 (d, J=8.6 Hz, 2H), 1.47 (d, J=6.5 Hz, 6H), 1.36 (d, J=4.0 Hz, 2H), 1.28 (dd, J=10.5, 5.3 Hz, 2H). LCMS (ESI) calcd, for C₄₁H₄₇N₁₄O₆S⁺ [M+H]⁺: 863.35, found, 863.4.

### Example 176: Preparation of 1-(5-((4-(1-isopropyl-6-((2-(4-methoxypiperidin-1-yl)pyrimidin-4-yl)amino)-1H-pyrazolo[4,3-c]pyridin-3-yl)piperazin-1-yl)methyl)-1-oxoisoindolin-2-yl)dihydropyrimidine-2,4 (1H,3H)-dione (GT-06034)

With reference to the method of Step 2 in Scheme T-1, the target compound (GT-06034) was obtained (yellow solid, 26 mg, yield 33.13%). ¹H NMR (400 MHz, DMSO-d₆) δ 11.93 (s, 1H), 11.54 (s, 1H), 10.67 (d, J=12.2 Hz, 1H), 9.12 (d, J=6.7 Hz, 1H), 8.08 (s, 1H), 8.02-7.93 (m, 2H), 7.90-7.82 (m, 2H), 6.66 (s, 1H), 4.89-4.78 (m, 1H), 4.69 (dt, J=12.7, 6.3 Hz, 1H), 4.57 (s, 3H), 4.10-4.02 (m, 4H), 3.53 (dd, J=35.9, 32.7 Hz, 8H), 3.28 (s, 5H), 2.97-2.72 (m, 2H), 2.01-1.85 (m, 2H), 1.59 (d, J=8.3 Hz, 2H), 1.43 (d, J=6.6 Hz, 6H). LCMS (ESI) calcd, for C₃₆H₄₅N₁₂O₄⁺ [M+H]⁺: 709.37, found, 709.4.

### Example 177: Preparation of 2-(2,4-dioxotetrahydropyrimidin-1(2H)-yl)-5-((4-(1-isopropyl-6-((2-(4-methoxypiperidin-1-yl)pyrimidin-4-yl)amino)-1H-pyrazolo[4,3-c]pyridin-3-yl)piperazin-1-yl)methyl)isoindoline-1,3-dione (GT-06127)

With reference to the method of Step 2 in Scheme T-1, the target compound (GT-06127) was obtained (yellow solid, 28 mg, yield 34.99%). ¹H NMR (400 MHz, DMSO-d₆) δ 12.23-11.43 (m, 1H), 11.31 (s, 1H), 10.78 (d, J=118.2 Hz, 1H), 9.03 (d, J=3.2 Hz, 1H), 8.46-8.06 (m, 2H), 8.01-7.86 (m, 2H), 6.63 (s, 1H), 4.73-4.46 (m, 3H), 4.05 (d, J=8.4 Hz, 4H), 3.85-3.67 (m, 8H), 3.29 (s, 6H), 2.81 (dt, J=42.1, 6.7 Hz, 2H), 1.95 (s, 2H), 1.60 (d, J=7.8 Hz, 2H), 1.43 (d, J=6.5 Hz, 6H). LCMS (ESI) calcd, for C₃₆H₄₃N₁₂O₅⁺ [M+H]⁺: 723.35, found, 723.4.

### Example 178: Preparation of 2-(2,4-dioxotetrahydropyrimidin-1(2H)-yl)-5-(4-((4-(1-isopropyl-6-((2-(4-methoxypiperidin-1-yl)pyrimidin-4-yl)amino)-1H-pyrazolo[4,3-c]pyridin-3-yl)piperazin-1-yl)methyl)piperidin-1-yl)isoindoline-1,3-dione (GT-06075)

With reference to the method of Scheme T-3, the target compound (GT-06075) was obtained (yellow solid, 20 mg, yield 22.41%). ¹H NMR (400 MHz, DMSO-d₆) δ 11.17 (s, 1H), 10.85 (s, 1H), 10.39 (s, 1H), 9.03 (s, 1H), 8.14 (s, 1H), 7.97 (d, J=7.0 Hz, 1H), 7.74 (d, J=8.7 Hz, 1H), 7.43 (s, 1H), 7.33 (d, J=8.8 Hz, 1H), 6.60 (s, 1H), 4.71-4.60 (m, 1H), 4.17-4.01 (m, 5H), 3.79 (t, J=6.8 Hz, 2H), 3.63 (s, 4H), 3.54 (d, J=15.0 Hz, 4H), 3.31 (s, 6H), 3.08 (dd, J=27.0, 14.3 Hz, 4H), 2.84 (t, J=6.9 Hz, 2H), 2.23 (s, 1H), 1.96 (d, J=9.1 Hz, 4H), 1.63 (s, 2H), 1.45 (d, J=6.6 Hz, 6H), 1.28 (dd, J=27.9, 12.1 Hz, 4H). LCMS (ESI) calcd, for C₄₁H₅₂N₁₃O₅⁺ [M+H]⁺: 806.42, found, 806.5.

### Example 179: Preparation of 2-(2,4-dioxotetrahydropyrimidin-1(2H)-yl)-4-(4-((4-(1-isopropyl-6-((2-(4-methoxypiperidin-1-yl)pyrimidin-4-yl)amino)-1H-pyrazolo[4,3-c]pyridin-3-yl)piperazin-1-yl)methyl)piperidin-1-yl)isoindoline-1,3-dione (GT-06035)

With reference to the method of Scheme T-3, the target compound (GT-06035) was obtained (yellow solid, 15 mg, yield 16.81%). ¹H NMR (400 MHz, DMSO-d₆) δ 11.40 (s, 1H), 10.85 (s, 2H), 9.10 (s, 1H), 8.11 (s, 1H), 7.97 (d, J=7.0 Hz, 1H), 7.80-7.70 (m, 1H), 7.50 (s, 1H), 7.40 (dd, J=10.8, 8.0 Hz, 1H), 6.65 (s, 1H), 4.73-4.64 (m, 1H), 4.06 (d, J=10.2 Hz, 4H), 3.85-3.76 (m, 8H), 3.43 (d, J=7.1 Hz, 4H), 3.23 (d, J=53.4 Hz, 9H), 2.99 (d, J=11.3 Hz, 1H), 2.86-2.77 (m, 2H), 2.00 (d, J=32.7 Hz, 4H), 1.60 (d, J=8.0 Hz, 2H), 1.45 (d, J=6.5 Hz, 7H). LCMS (ESI) calcd, for C₄₁H₅₂N₁₃O₅⁺ [M+H]⁺: 806.42, found, 806.5.

### Example 180: Preparation of 1-(5-(((1-(1-isopropyl-6-((2-(4-methoxypiperidin-1-yl)pyrimidin-4-yl)amino)-1H-pyrazolo[4,3-c]pyridin-3-yl)piperidin-4-yl)(methyl)amino)methyl)-1-oxoisoindolin-2-yl)dihydropyrimidine-2,4 (1H,3H)-dione (GT-06109)

With reference to the method of Step 2 in Scheme T-1, the target compound (GT-06109) was obtained (yellow solid, 26 mg, yield 33.85%). ¹H NMR (400 MHz, DMSO-d₆) δ 11.43 (d, J=72.0 Hz, 2H), 10.70 (s, 1H), 9.11 (s, 1H), 8.08 (s, 1H), 7.99 (d, J=7.0 Hz, 2H), 7.87 (t, J=7.1 Hz, 2H), 6.67 (s, 1H), 4.81 (dt, J=24.9, 12.6 Hz, 1H), 4.67 (dd, J=18.7, 12.0 Hz, 3H), 4.39 (dd, J=12.7, 6.6 Hz, 1H), 4.19 (d, J=12.1 Hz, 2H), 4.11-4.04 (m, 2H), 3.80 (s, 2H), 3.53 (dd, J=11.6, 8.0 Hz, 3H), 3.30 (s, 3H), 2.98 (d, J=12.1 Hz, 2H), 2.82 (d, J=31.5 Hz, 2H), 2.63 (d, J=4.3 Hz, 3H), 2.37 (s, 2H), 1.97 (d, J=3.9 Hz, 4H), 1.61 (d, J=8.3 Hz, 2H), 1.45 (d, J=6.6 Hz, 6H). LCMS (ESI) calcd, for C₃₈H₄₉N₁₂O₄⁺ [M+H]⁺: 737.40, found, 737.5.

### Example 181: Preparation of 2-(2,4-dioxotetrahydropyrimidin-1(2H)-yl)-5-(((1-(1-isopropyl-6-((2-(4-methoxypiperidin-1-yl)pyrimidin-4-yl)amino)-1H-pyrazolo[4,3-c]pyridin-3-yl)piperidin-4-yl)(methyl)amino)methyl)isoindoline-1,3-dione (GT-06110)

With reference to the method of Step 2 in Scheme T-1, the target compound (GT-06110) was obtained (yellow solid, 36 mg, yield 45.99%). ¹H NMR (400 MHz, DMSO-d₆) δ 11.32 (s, 1H), 10.92 (d, J=6.9 Hz, 1H), 10.55 (dd, J=59.6, 4.4 Hz, 1H), 9.04 (s, 1H), 8.45-8.19 (m, 1H), 8.10 (dd, J=22.9, 15.1 Hz, 1H), 8.02-7.71 (m, 3H), 6.65 (s, 1H), 4.65 (td, J=12.7, 6.1 Hz, 2H), 4.41 (d, J=55.5 Hz, 1H), 4.18 (d, J=11.3 Hz, 2H), 4.03 (s, 2H), 3.79 (s, 5H), 3.29 (s, 4H), 2.99 (s, 2H), 2.86-2.75 (m, 2H), 2.63 (s, 3H), 2.30 (s, 2H), 1.97 (d, J=7.2 Hz, 4H), 1.61 (d, J=8.4 Hz, 2H), 1.43 (d, J=6.6 Hz, 6H). LCMS (ESI) calcd, for C₃₈H₄₇N₁₂O₅⁺ [M+H]⁺: 751.38, found, 751.4.

### Example 182: Preparation of 2-(2,4-dioxotetrahydropyrimidin-1(2H)-yl)-5-(4-(((1-(1-isopropyl-6-((2-(4-methoxypiperidin-1-yl)pyrimidin-4-yl)amino)-1H-pyrazolo[4,3-c]pyridin-3-yl)piperidin-4-yl)(methyl)amino)methyl)piperidin-1-yl)isoindoline-1,3-dione (GT-06112)

With reference to the method of Scheme T-3, the target compound (GT-06112) was obtained (yellow solid, 26 mg, yield 29.91%). ¹H NMR (400 MHz, DMSO-d₆) δ 11.50 (s, 1H), 10.84 (s, 2H), 10.51-10.23 (m, 2H), 9.63 (d, J=3.3 Hz, 1H), 9.08 (s, 1H), 7.99 (d, J=7.0 Hz, 2H), 7.71 (ddd, J=13.7, 7.9, 5.9 Hz, 3H), 7.53-7.24 (m, 5H), 7.05 (dd, J=23.9, 11.9 Hz, 1H), 6.66 (s, 1H), 4.72-4.65 (m, 1H), 4.21-4.10 (m, 6H), 3.55 (d, J=3.7 Hz, 8H), 3.30 (d, J=3.3 Hz, 3H), 3.16-3.00 (m, 5H), 2.84-2.73 (m, 6H), 2.21 (d, J=32.0 Hz, 4H), 1.97-1.82 (m, 7H), 1.44 (d, J=6.5 Hz, 6H), 1.37-1.21 (m, 2H). LCMS (ESI) calcd, for C₄₃H₅₆N₁₃O₅⁺ [M+H]⁺: 834.45, found, 834.5.

### Example 183: Preparation of 2-(2,4-dioxotetrahydropyrimidin-1(2H)-yl)-4-(4-(((1-(1-isopropyl-6-((2-(4-methoxypiperidin-1-yl)pyrimidin-4-yl)amino)-1H-pyrazolo[4,3-c]pyridin-3-yl)piperidin-4-yl)(methyl)amino)methyl)piperidin-1-yl)isoindoline-1,3-dione (GT-06119)

With reference to the method of Scheme T-3, the target compound (GT-06119) was obtained (yellow solid, 16 mg, yield 18.40%). ¹H NMR (400 MHz, DMSO-d₆) δ 11.36 (s, 1H), 10.86 (s, 3H), 10.17 (s, 1H), 9.67 (s, 1H), 9.05 (s, 1H), 7.98 (d, J=7.1 Hz, 1H), 7.78-7.70 (m, 3H), 7.61-7.48 (m, 1H), 7.44-7.35 (m, 6H), 6.65 (s, 1H), 4.66 (s, 1H), 4.17 (d, J=12.0 Hz, 2H), 4.04 (s, 2H), 3.82-3.76 (m, 7H), 3.31 (d, J=2.4 Hz, 3H), 3.20 (d, J=6.6 Hz, 2H), 3.02 (dd, J=19.5, 9.1 Hz, 6H), 2.82 (dd, J=11.4, 4.5 Hz, 6H), 2.23 (d, J=19.6 Hz, 2H), 1.95 (d, J=22.8 Hz, 7H), 1.65 (dd, J=31.5, 10.7 Hz, 4H), 1.44 (d, J=6.6 Hz, 6H). LCMS (ESI) calcd. for C₄₃H₅₆N₁₃O₅⁺ [M+H]⁺: 834.45, found, 834.5.

### Example 184: Preparation of 2-(6,7-dihydro-5H-pyrrolo[1,2-c]imidazol-1-yl)-2-(6-(4-(4-((2-(2,4-dioxotetrahydropyrimidin-1(2H)-yl)-1-oxoisoindolin-5-yl)methyl)piperazin-1-yl)phenyl)-4-fluoro-1-oxoisoindolin-2-yl)-N-(thiazol-2-yl)acetamide (GT-06159)

With reference to the method of Step 2 in Scheme T-1, the target compound (GT-06159) was obtained (yellow solid, 26 mg, yield 44.48%). ¹H NMR (400 MHz, DMSO-d₆) δ 11.45 (s, 1H), 10.70 (s, 2H), 9.33 (s, 1H), 9.04 (d, J=17.1 Hz, 2H), 7.95 (s, 2H), 7.91-7.78 (m, 8H), 7.72 (dd, J=8.8, 2.2 Hz, 4H), 7.53 (d, J=3.6 Hz, 1H), 7.32 (d, J=3.6 Hz, 1H), 7.28 (d, J=4.4 Hz, 1H), 7.09 (d, J=8.8 Hz, 4H), 6.91 (d, J=4.4 Hz, 1H), 6.39 (s, 1H), 6.17 (s, 1H), 4.81 (t, J=15.8 Hz, 3H), 4.54 (s, 4H), 4.26 (d, J=3.8 Hz, 2H), 3.91 (s, 3H), 3.24-3.18 (m, 4H), 3.03-2.97 (m, 1H), 2.90-2.84 (m, 3H), 2.79 (d, J=5.6 Hz, 3H), 2.62-2.56 (m, 3H). LCMS (ESI) calcd. for C₄₂H₄₀FN₁₀O₅S⁺ [M+H]⁺: 815.29, found, 815.3.

### Example 185: Preparation of 2-(6,7-dihydro-5H-pyrrolo[1,2-c]imidazol-1-yl)-2-(6-(4-(4-((2-(2,4-dioxotetrahydropyrimidin-1(2H)-yl)-1,3-dioxoisoindolin-5-yl)methyl)piperazin-1-yl)phenyl)-4-fluoro-1-oxoisoindolin-2-yl)-N-(thiazol-2-yl)acetamide (GT-06160)

With reference to the method of Step 2 in Scheme T-1, the target compound (GT-06160) was obtained (yellow solid, 12 mg, yield 20.18%). ¹H NMR (400 MHz, DMSO-d₆) δ 11.52 (s, 2H), 10.72-10.42 (m, 1H), 9.06 (d, J=16.9 Hz, 1H), 8.42-8.23 (m, 1H), 8.19-7.97 (m, 1H), 7.82 (dd, J=7.1, 1.9 Hz, 2H), 7.74 (t, J=7.9 Hz, 2H), 7.53 (d, J=3.6 Hz, 1H), 7.31 (dd, J=18.3, 4.0 Hz, 1H), 7.01 (dd, J=74.0, 6.7 Hz, 2H), 6.29 (d, J=89.7 Hz, 1H), 4.86-4.71 (m, 1H), 4.63 (s, 1H), 4.59-4.48 (m, 2H), 4.26 (dd, J=8.7, 4.9 Hz, 2H), 3.94 (s, 2H), 3.84 (d, J=6.8 Hz, 1H), 3.71-3.62 (m, 4H), 3.17 (s, 4H), 2.99 (dd, J=15.4, 8.1 Hz, 1H), 2.87 (t, J=6.9 Hz, 2H), 2.76 (t, J=6.8 Hz, 1H), 2.60 (dd, J=15.8, 6.2 Hz, 2H). LCMS (ESI) calcd, for C₄₂H₃₈FN₁₀O₆S⁺ [M+H]⁺: 829.27, found, 829.3.

### Example 186: Preparation of 2-(6,7-dihydro-5H-pyrrolo[1,2-c]imidazol-1-yl)-2-(6-(4-(4-((1-(2-(2,4-dioxotetrahydropyrimidin-1(2H)-yl)-1,3-dioxoisoindolin-5-yl)piperidin-4-yl)methyl)piperazin-1-yl)phenyl)-4-fluoro-1-oxoisoindolin-2-yl)-N-(thiazol-2-yl)acetamide (GT-06161)

With reference to the method of Scheme T-3, the target compound (GT-06161) was obtained (yellow solid, 36 mg, yield 44.03%). ¹H NMR (400 MHz, DMSO-d₆) δ 10.85 (s, 1H), 10.75 (s, 1H), 9.35 (s, 1H), 9.07 (d, J=18.4 Hz, 1H), 7.82 (dd, J=8.3, 5.8 Hz, 2H), 7.74 (dd, J=8.6, 3.9 Hz, 3H), 7.53 (d, J=3.6 Hz, 1H), 7.42 (d, J=1.8 Hz, 1H), 7.32 (dd, J=11.0, 7.0 Hz, 2H), 7.13 (d, J=8.8 Hz, 2H), 6.29 (d, J=89.6 Hz, 2H), 4.79 (t, J=14.1 Hz, 1H), 4.52 (d, J=17.0 Hz, 1H), 4.26 (t, J=6.9 Hz, 2H), 4.12 (d, J=12.9 Hz, 2H), 3.92 (s, 2H), 3.80 (d, J=6.8 Hz, 2H), 3.62 (s, 4H), 3.07 (d, J=13.6 Hz, 4H), 2.86 (dt, J=14.5, 6.6 Hz, 4H), 2.65-2.54 (m, 3H), 2.24 (s, 1H), 1.98 (d, J=11.0 Hz, 2H), 1.30 (d, J=11.4 Hz, 2H). LCMS (ESI) calcd, for C₄₇H₄₇FN₁₁O₆S⁺ [M+H]⁺: 912.34, found, 912.4.

### Example 187: Preparation of 2-(6,7-dihydro-5H-pyrrolo[1,2-c]imidazol-1-yl)-2-(6-(4-(4-((1-(2-(2,4-dioxotetrahydropyrimidin-1(2H)-yl)-1,3-dioxoisoindolin-4-yl)piperidin-4-yl)methyl)piperazin-1-yl)phenyl)-4-fluoro-1-oxoisoindolin-2-yl)-N-(thiazol-2-yl)acetamide (GT-06162)

With reference to the method of Scheme T-3, the target compound (GT-06162) was obtained (yellow solid, 20 mg, yield 24.46%). ¹H NMR (400 MHz, DMSO-d₆) δ 12.83 (s, 1H), 10.85 (s, 2H), 10.39 (s, 1H), 8.96 (s, 1H), 7.87-7.71 (m, 4H), 7.53 (d, J=3.6 Hz, 1H), 7.43-7.37 (m, 2H), 7.32 (d, J=3.6 Hz, 1H), 7.13 (d, J=8.8 Hz, 2H), 6.37 (s, 1H), 4.64 (dd, J=121.9, 17.2 Hz, 2H), 4.25 (dd, J=17.1, 10.3 Hz, 2H), 3.92 (d, J=12.9 Hz, 2H), 3.84-3.63 (m, 6H), 3.16 (s, 4H), 2.98-2.78 (m, 6H), 2.65-2.53 (m, 3H), 2.05 (d, J=55.9 Hz, 4H), 1.72 (d, J=12.0 Hz, 1H), 1.48 (s, 2H). LCMS (ESI) calcd, for C₄₇H₄₇FN₁₁O₆S⁺ [M+H]⁺: 912.34, found, 912.4.

### Example 188: Preparation of 2-(6,7-dihydro-5H-pyrrolo[1,2-c]imidazol-1-yl)-2-(6-(4-(4-(4-((2-(2,4-dioxotetrahydropyrimidin-1(2H)-yl)-1-oxoisoindolin-5-yl)methyl)piperazin-1-yl)piperidin-1-yl)phenyl)-4-fluoro-1-oxoisoindolin-2-yl)-N-(thiazol-2-yl)acetamide (GT-06241)

With reference to the method of Step 2 in Scheme T-1, the target compound (GT-06241) was obtained (yellow solid, 16 mg, yield 25.37%). ¹H NMR (400 MHz, DMSO-d₆) δ 12.23 (s, 1H), 10.69 (s, 1H), 9.40 (s, 1H), 9.08 (d, J=19.5 Hz, 1H), 7.96 (s, 1H), 7.90-7.79 (m, 4H), 7.76-7.67 (m, 2H), 7.53 (d, J=3.6 Hz, 1H), 7.33 (d, J=3.6 Hz, 1H), 7.29 (d, J=4.4 Hz, 1H), 7.18 (d, J=8.5 Hz, 2H), 6.91 (d, J=4.4 Hz, 1H), 6.29 (d, J=91.0 Hz, 1H), 4.91-4.68 (m, 3H), 4.66-4.52 (m, 4H), 4.27 (d, J=3.5 Hz, 2H), 3.99 (s, 2H), 3.79 (d, J=6.8 Hz, 8H), 3.47-3.43 (m, 2H), 2.87 (d, J=10.6 Hz, 6H), 2.55 (s, 3H), 2.21 (d, J=9.5 Hz, 2H), 1.95-1.79 (m, 2H). LCMS (ESI) calcd, for C₄₇H₄₉FN₁₁O₅S⁺ [M+H]⁺: 898.36, found, 898.4.

### Example 189: Preparation of 2-(6,7-dihydro-5H-pyrrolo[1,2-c]imidazol-1-yl)-2-(6-(4-(4-(4-((2-(2,4-dioxotetrahydropyrimidin-1(2H)-yl)-1,3-dioxoisoindolin-5-yl)methyl)piperazin-1-yl)piperidin-1-yl)phenyl)-4-fluoro-1-oxoisoindolin-2-yl)-N-(thiazol-2-yl)acetamide (GT-06242)

With reference to the method of Step 2 in Scheme T-1, the target compound (GT-06242) was obtained (yellow solid, 15 mg, yield 23.42%). ¹H NMR (400 MHz, DMSO) δ 10.74 (dd, J=142.2, 30.7 Hz, 1H), 9.06 (d, J=18.1 Hz, 1H), 8.33-8.06 (m, 1H), 8.00-7.88 (m, 1H), 7.80 (t, J=7.2 Hz, 2H), 7.72 (t, J=9.0 Hz, 2H), 7.31 (dd, J=17.5, 4.0 Hz, 1H), 7.14 (d, J=8.6 Hz, 2H), 6.28 (d, J=87.8 Hz, 1H), 4.76 (dd, J=29.1, 15.9 Hz, 1H), 4.49 (dd, J=20.4, 14.0 Hz, 3H), 4.27 (d, J=3.5 Hz, 2H), 3.98 (d, J=11.7 Hz, 2H), 3.85-3.81 (m, 2H), 3.67 (s, 7H), 3.26-3.16 (m, 4H), 2.89-2.81 (m, 4H), 2.78-2.74 (m, 1H), 2.63-2.56 (m, 2H), 2.19 (s, 2H), 1.84 (d, J=8.5 Hz, 2H). LCMS (ESI) calcd, for C₄₇H₄₇FN₁₁O₆S⁺ [M+H]⁺: 912.34, found, 912.3.

### Example 190: Preparation of 2-(6,7-dihydro-5H-pyrrolo[1,2-c]imidazol-1-yl)-2-(6-(4-(4-(1-((2-(2,4-dioxotetrahydropyrimidin-1(2H)-yl)-1-oxoisoindolin-5-yl)methyl)piperidin-4-yl)piperazin-1-yl)phenyl)-4-fluoro-1-oxoisoindolin-2-yl)-N-(thiazol-2-yl)acetamide (GT-06324)

With reference to the method of Step 2 in Scheme T-1, the target compound (GT-06324) was obtained (yellow solid, 23 mg, yield 32.82%). ¹H NMR (400 MHz, DMSO-d₆) δ 11.45 (d, J=113.7 Hz, 2H), 10.70 (s, 1H), 9.34 (s, 1H), 9.06 (d, J=16.4 Hz, 1H), 7.82 (ddd, J=35.9, 21.7, 7.8 Hz, 9H), 7.53 (d, J=3.5 Hz, 1H), 7.33 (d, J=3.5 Hz, 1H), 7.11 (d, J=8.3 Hz, 3H), 6.40 (s, 1H), 4.81 (dd, J=16.6, 9.5 Hz, 2H), 4.48 (d, J=10.6 Hz, 3H), 4.26 (s, 2H), 3.96 (s, 2H), 3.80 (d, J=6.6 Hz, 2H), 3.57 (s, 5H), 3.25-3.16 (m, 4H), 3.04 (s, 2H), 2.91-2.78 (m, 3H), 2.60 (dd, J=21.2, 6.2 Hz, 3H), 2.38 (s, 2H), 2.26 (s, 2H). LCMS (ESI) calcd, for C₄₇H₄₉FN₁₁O₅S⁺ [M+H]⁺: 898.36, found, 898.3.

### Example 191: Preparation of 2-(6,7-dihydro-5H-pyrrolo[1,2-c]imidazol-1-yl)-2-(6-(4-(4-(1-((2-(2,4-dioxotetrahydropyrimidin-1(2H)-yl)-1,3-dioxoisoindolin-5-yl)methyl)piperidin-4-yl)piperazin-1-yl)phenyl)-4-fluoro-1-oxoisoindolin-2-yl)-N-(thiazol-2-yl)acetamide (GT-06325)

With reference to the method of Step 2 in Scheme T-1, the target compound (GT-06325) was obtained (yellow solid, 18 mg, yield 19.16%). ¹H NMR (400 MHz, DMSO-d₆) δ 11.59 (s, 2H), 10.76 (dd, J=97.0, 27.7 Hz, 1H), 9.33 (s, 2H), 9.06 (d, J=16.1 Hz, 1H), 7.82 (s, 2H), 7.76-7.70 (m, 3H), 7.31 (dd, J=19.0, 4.0 Hz, 1H), 7.12 (d, J=8.1 Hz, 3H), 6.91 (d, J=4.4 Hz, 1H), 6.29 (d, J=89.4 Hz, 1H), 4.73 (d, J=17.3 Hz, 1H), 4.59-4.41 (m, 4H), 4.27 (t, J=7.0 Hz, 3H), 3.95 (s, 3H), 3.86-3.82 (m, 2H), 3.65 (s, 2H), 3.04-2.97 (m, 4H), 2.89-2.76 (m, 5H), 2.60 (d, J=7.1 Hz, 2H), 2.39 (s, 3H), 2.23 (s, 2H). LCMS (ESI) calcd, for C₄₇H₄₇FN₁₁O₆S⁺ [M+H]⁺: 912.34, found, 912.4.

### Example 192: Preparation of 2-(6,7-dihydro-5H-pyrrolo[1,2-c]imidazol-1-yl)-2-(6-(4-(4-(4-((2-(2,4-dioxotetrahydropyrimidin-1(2H)-yl)-1-oxoisoindolin-5-yl)methyl)piperazin-1-yl)-3,3-difluoropiperidin-1-yl)phenyl)-4-fluoro-1-oxoisoindolin-2-yl)-N-(thiazol-2-yl)acetamide (GT-06315)

With reference to the method of Step 2 in Scheme T-1, the target compound (GT-06315) was obtained (yellow solid, 37 mg, yield 59.57%). ¹H NMR (400 MHz, DMSO-d₆) δ 11.42 (s, 1H), 10.69 (d, J=7.1 Hz, 1H), 9.08 (d, J=19.4 Hz, 1H), 7.93 (s, 1H), 7.82 (ddd, J=9.7, 8.2, 5.9 Hz, 4H), 7.67 (d, J=8.8 Hz, 2H), 7.53 (d, J=3.6 Hz, 1H), 7.33 (d, J=3.6 Hz, 1H), 7.09 (d, J=8.9 Hz, 2H), 6.41 (s, 1H), 4.81 (dd, J=16.7, 5.9 Hz, 2H), 4.57 (d, J=21.9 Hz, 2H), 4.48 (d, J=16.8 Hz, 2H), 4.26 (t, J=7.0 Hz, 2H), 4.07 (s, 1H), 3.96 (d, J=12.5 Hz, 1H), 3.80 (d, J=6.8 Hz, 2H), 3.68 (s, 3H), 3.48-3.44 (m, 1H), 3.29 (s, 2H), 3.08 (d, J=13.4 Hz, 6H), 2.95-2.83 (m, 3H), 2.80-2.73 (m, 1H), 2.66-2.56 (m, 2H), 1.93 (d, J=30.1 Hz, 2H). LCMS (ESI) calcd. for C₄₇H₄₇F₃N₁₁O₅S⁺ [M+H]⁺: 934.34, found, 934.4.

### Example 193: Preparation of 2-(6,7-dihydro-5H-pyrrolo[1,2-c]imidazol-1-yl)-2-(6-(4-(4-(4-((2-(2,4-dioxotetrahydropyrimidin-1(2H)-yl)-1,3-dioxoisoindolin-5-yl)methyl)piperazin-1-yl)-3,3-difluoropiperidin-1-yl)phenyl)-4-fluoro-1-oxoisoindolin-2-yl)-N-(thiazol-2-yl)acetamide (GT-06316)

With reference to the method of Step 2 in Scheme T-1, the target compound (GT-06316) was obtained (yellow solid, 25 mg, yield 39.66%). ¹H NMR (400 MHz, DMSO-d₆) δ 10.92 (s, 2H), 9.07 (s, 1H), 8.34-8.06 (m, 2H), 7.79 (d, J=7.3 Hz, 2H), 7.66 (d, J=8.7 Hz, 3H), 7.52 (d, J=3.6 Hz, 1H), 7.32 (d, J=3.6 Hz, 1H), 7.08 (d, J=8.8 Hz, 2H), 6.38 (s, 1H), 4.78 (d, J=17.2 Hz, 1H), 4.50 (d, J=17.4 Hz, 3H), 4.25 (t, J=6.9 Hz, 2H), 4.05 (s, 1H), 3.95 (s, 1H), 3.82 (d, J=6.7 Hz, 2H), 3.30-3.25 (m, 7H), 3.08-3.02 (m, 5H), 2.91-2.84 (m, 3H), 2.75 (s, 1H), 2.56 (d, J=6.5 Hz, 2H), 1.87 (s, 2H). LCMS (ESI) calcd, for C₄₇H₄₅F₃N₁₁O₆S⁺ [M+H]⁺: 948.32, found, 948.3.

### Example 194: Preparation of 2-(6,7-dihydro-5H-pyrrolo[1,2-c]imidazol-1-yl)-2-(6-(4-((1R,4R)-5-((2-(2,4-dioxotetrahydropyrimidin-1(2H)-yl)-1-oxoisoindolin-5-yl)methyl)-2,5-diazabicyclo[2.2.1]heptan-2-yl)phenyl)-4-fluoro-1-oxoisoindolin-2-yl)-N-(thiazol-2-yl)acetamide (GT-06172)

With reference to the method of Step 2 in Scheme T-1, the target compound (GT-06172) was obtained (yellow solid, 16 mg, yield 27.56%). ¹H NMR (400 MHz, DMSO-d₆) δ 10.91 (s, 1H), 10.67 (d, J=13.4 Hz, 2H), 9.04 (d, J=13.6 Hz, 1H), 8.87-8.60 (m, 1H), 8.00 (s, 1H), 7.88-7.77 (m, 4H), 7.70-7.63 (m, 2H), 7.53 (d, J=3.6 Hz, 1H), 7.33 (d, J=3.6 Hz, 1H), 6.74 (d, J=8.6 Hz, 2H), 6.38 (s, 1H), 5.81 (s, 1H), 4.84-4.69 (m, 4H), 4.53 (dd, J=23.6, 17.2 Hz, 4H), 4.26 (s, 2H), 3.81-3.75 (m, 4H), 3.65 (d, J=9.7 Hz, 2H), 3.24 (s, 3H), 2.92-2.85 (m, 2H), 2.78 (s, 1H), 2.61 (d, J=10.8 Hz, 2H), 2.20 (d, J=10.6 Hz, 1H). LCMS (ESI) calcd. for C₄₃H₄₀FN₁₀O₅S⁺ [M+H]⁺: 827.29, found, 827.3.

### Example 195: Preparation of 2-(6,7-dihydro-5H-pyrrolo[1,2-c]imidazol-1-yl)-2-(6-(4-((1R,4R)-5-((2-(2,4-dioxotetrahydropyrimidin-1(2H)-yl)-1,3-dioxoisoindolin-5-yl)methyl)-2,5-diazabicyclo[2.2.1]heptan-2-yl)phenyl)-4-fluoro-1-oxoisoindolin-2-yl)-N-(thiazol-2-yl)acetamide (GT-06173)

With reference to the method of Step 2 in Scheme T-1, the target compound (GT-06173) was obtained (yellow solid, 17 mg, yield 28.79%). ¹H NMR (400 MHz, DMSO-d₆) δ 10.98-10.87 (m, 1H), 10.53 (dd, J=47.7, 6.9 Hz, 1H), 9.00 (s, 1H), 8.02-7.89 (m, 2H), 7.78 (s, 2H), 7.66 (d, J=8.3 Hz, 2H), 7.53 (d, J=3.6 Hz, 1H), 7.33 (d, J=3.5 Hz, 1H), 6.76 (s, 1H), 6.37 (s, 1H), 4.78 (d, J=17.4 Hz, 2H), 4.49 (d, J=17.3 Hz, 2H), 4.26 (d, J=6.3 Hz, 2H), 3.82 (t, J=6.6 Hz, 2H), 3.67 (d, J=12.5 Hz, 2H), 3.46 (s, 2H), 3.17 (s, 6H), 2.85 (d, J=6.5 Hz, 2H), 2.60 (d, J=14.2 Hz, 2H), 2.22 (s, 1H). LCMS (ESI) calcd, for C₄₃H₃₈FN₁₀O₆S⁺ [M+H]⁺: 841.27, found, 841.3.

### Example 196: Preparation of 2-(6,7-dihydro-5H-pyrrolo[1,2-c]imidazol-1-yl)-2-(6-(4-((1R,4R)-5-((1-(2-(2,4-dioxotetrahydropyrimidin-1(2H)-yl)-1,3-dioxoisoindolin-5-yl)piperidin-4-yl)methyl)-2,5-diazabicyclo[2.2.1]heptan-2-yl)phenyl)-4-fluoro-1-oxoisoindolin-2-yl)-N-(thiazol-2-yl)acetamide (GT-06174)

With reference to the method of Step 2 in Scheme T-1, the target compound (GT-06174) was obtained (yellow solid, 22 mg, yield 33.91%). ¹H NMR (400 MHz, DMSO-d₆) δ 11.33 (s, 1H), 10.83 (d, J=4.1 Hz, 1H), 10.01 (s, 1H), 9.06 (d, J=17.8 Hz, 1H), 7.90-7.78 (m, 2H), 7.70 (dd, J=10.7, 8.7 Hz, 2H), 7.52 (d, J=3.6 Hz, 1H), 7.38 (d, J=4.6 Hz, 1H), 7.34-7.27 (m, 2H), 6.77 (t, J=7.8 Hz, 1H), 6.40 (d, J=5.4 Hz, 1H), 4.75 (dd, J=27.1, 13.1 Hz, 2H), 4.50 (d, J=16.9 Hz, 2H), 4.26 (t, J=6.8 Hz, 2H), 4.10 (d, J=12.2 Hz, 2H), 3.77 (dd, J=8.9, 4.6 Hz, 3H), 3.62 (d, J=17.0 Hz, 6H), 3.26-3.20 (m, 2H), 3.04-2.95 (m, 2H), 2.83 (dd, J=14.9, 7.6 Hz, 3H), 2.60 (dd, J=21.7, 6.5 Hz, 2H), 2.38 (d, J=9.8 Hz, 1H), 2.15 (d, J=10.3 Hz, 1H), 1.99-1.84 (m, 2H), 1.25 (s, 2H). LCMS (ESI) calcd, for C₄₈H₄₇FN₁₁O₆S⁺ [M+H]⁺: 924.34, found, 924.4.

### Example 197: Preparation of 2-(6,7-dihydro-5H-pyrrolo[1,2-c]imidazol-1-yl)-2-(6-(4-((1R,4R)-5-((1-(2-(2,4-dioxotetrahydropyrimidin-1(2H)-yl)-1,3-dioxoisoindolin-4-yl)piperidin-4-yl)methyl)-2,5-diazabicyclo[2.2.1]heptan-2-yl)phenyl)-4-fluoro-1-oxoisoindolin-2-yl)-N-(thiazol-2-yl)acetamide (GT-06175)

With reference to the method of Step 2 in Scheme T-1, the target compound (GT-06175) was obtained (yellow solid, 6 mg, yield 9.25%). ¹H NMR (400 MHz, DMSO-d₆) δ 10.86 (s, 1H), 9.71 (s, 1H), 9.23 (s, 2H), 9.03 (d, J=13.5 Hz, 2H), 7.81 (d, J=5.4 Hz, 2H), 7.77-7.69 (m, 3H), 7.53 (d, J=3.6 Hz, 1H), 7.40 (dd, J=7.9, 4.6 Hz, 2H), 7.33 (d, J=3.6 Hz, 1H), 7.28 (d, J=4.4 Hz, 1H), 6.92 (d, J=4.4 Hz, 1H), 6.79 (t, J=9.2 Hz, 2H), 6.38 (s, 1H), 6.17 (s, 1H), 4.73 (dd, J=20.7, 12.4 Hz, 3H), 4.50 (d, J=16.2 Hz, 2H), 4.27 (d, J=4.1 Hz, 3H), 3.77 (s, 4H), 3.18-3.09 (m, 4H), 2.93-2.81 (m, 5H), 2.58 (d, J=7.1 Hz, 3H), 2.41 (d, J=10.4 Hz, 1H), 2.18 (d, J=9.7 Hz, 1H), 1.95 (s, 4H), 1.44 (s, 2H). LCMS (ESI) calcd, for C₄₈H₄₇FN₁₁O₆S⁺ [M+H]⁺: 924.34, found, 924.4.

### Example 198: Preparation of 2-(6,7-dihydro-5H-pyrrolo[1,2-c]imidazol-1-yl)-2-(6-(4-(8-((2-(2,4-dioxotetrahydropyrimidin-1(2H)-yl)-1-oxoisoindolin-5-yl)methyl)-3,8-diazabicyclo[3.2.1]octan-3-yl)phenyl)-4-fluoro-1-oxoisoindolin-2-yl)-N-(thiazol-2-yl)acetamide (GT-06250)

With reference to the method of Step 2 in Scheme T-1, the target compound (GT-06250) was obtained (yellow solid, 25 mg, yield 38.56%). ¹H NMR (400 MHz, DMSO) δ 11.75 (s, 1H), 10.70 (s, 1H), 9.58-9.23 (m, 1H), 9.06 (d, J=17.5 Hz, 1H), 8.13 (s, 1H), 8.02 (d, J=7.8 Hz, 1H), 7.89 (d, J=7.8 Hz, 1H), 7.79 (dd, J=9.4, 5.6 Hz, 2H), 7.70 (d, J=8.6 Hz, 2H), 7.53 (d, J=3.6 Hz, 1H), 7.31 (dd, J=17.2, 4.0 Hz, 1H), 7.00 (t, J=10.3 Hz, 2H), 6.29 (d, J=89.5 Hz, 1H), 4.89-4.72 (m, 2H), 4.65-4.50 (m, 2H), 4.42 (s, 2H), 4.25 (d, J=6.9 Hz, 2H), 3.99 (s, 2H), 3.81 (t, J=6.8 Hz, 2H), 3.74 (d, J=11.3 Hz, 3H), 3.57 (s, 3H), 2.94-2.84 (m, 2H), 2.79 (s, 1H), 2.60 (dd, J=22.1, 6.7 Hz, 2H), 2.38 (s, 2H), 2.03 (d, J=8.3 Hz, 2H). LCMS (ESI) calcd. for C₄₄H₄₂FN₁₀O₅S⁺ [M+H]⁺: 841.30, found, 841.3.

### Example 199: Preparation of 2-(6,7-dihydro-5H-pyrrolo[1,2-c]imidazol-1-yl)-2-(6-(4-(8-((2-(2,4-dioxotetrahydropyrimidin-1(2H)-yl)-1,3-dioxoisoindolin-5-yl)methyl)-3,8-diazabicyclo[3.2.1]octan-3-yl)phenyl)-4-fluoro-1-oxoisoindolin-2-yl)-N-(thiazol-2-yl)acetamide (GT-06251)

With reference to the method of Step 2 in Scheme T-1, the target compound (GT-06251) was obtained (yellow solid, 17 mg, yield 25.79%). ¹H NMR (400 MHz, DMSO-d₆) δ 11.84 (s, 1H), 11.36 (d, J=69.5 Hz, 1H), 10.93 (s, 1H), 9.04 (d, J=16.0 Hz, 1H), 8.52 (s, 1H), 8.42 (d, J=7.8 Hz, 1H), 8.12 (d, J=7.7 Hz, 1H), 7.83-7.77 (m, 2H), 7.69 (d, J=8.4 Hz, 2H), 7.52 (d, J=3.6 Hz, 1H), 7.32 (d, J=3.6 Hz, 1H), 6.99 (d, J=8.5 Hz, 2H), 6.38 (s, 1H), 4.78 (d, J=17.1 Hz, 1H), 4.50 (d, J=16.9 Hz, 2H), 4.42 (d, J=10.4 Hz, 1H), 4.25 (t, J=7.0 Hz, 2H), 4.01 (s, 3H), 3.83 (t, J=6.8 Hz, 2H), 3.74 (s, 2H), 3.68-3.63 (m, 3H), 2.85 (d, J=6.8 Hz, 2H), 2.76 (t, J=6.8 Hz, 1H), 2.63-2.55 (m, 2H), 2.35 (d, J=16.2 Hz, 2H), 2.03 (d, J=8.2 Hz, 2H). LCMS (ESI) calcd. for C₄₄H₄₀FN₁₀O₆S⁺ [M+H]⁺: 855.28, found, 855.3.

### Example 200: Preparation of 7-cyclopentyl-2-((5-(4-((3-(2-(2,4-dioxotetrahydropyrimidin-1(2H)-yl)-1,3-dioxoisoindolin-5-yl)-3,6-diazabicyclo[3.1.1]heptan-6-yl)methyl)piperidin-1-yl)pyridin-2-yl)amino)-N,N-dimethyl-7H-pyrrolo[2,3-d]pyrimidine-6-carboxamide (GT-05820)

With reference to the method of Scheme T-3, the target compound (GT-05820) was obtained (yellow solid, 37 mg, yield 42.65%). ¹H NMR (400 MHz, DMSO-d₆) δ 11.48 (s, 1H), 10.86 (d, J=4.4 Hz, 1H), 9.00 (d, J=3.3 Hz, 1H), 8.11 (d, J=9.8 Hz, 1H), 7.88 (dd, J=20.2, 10.8 Hz, 2H), 7.57 (t, J=10.2 Hz, 1H), 7.26 (s, 1H), 7.17 (d, J=8.3 Hz, 1H), 6.82 (d, J=1.4 Hz, 1H), 4.92-4.75 (m, 1H), 4.67 (s, 1H), 4.12-3.93 (m, 3H), 3.86-3.66 (m, 5H), 3.50 (s, 2H), 3.06 (s, 6H), 2.91-2.68 (m, 6H), 2.31 (s, 2H), 1.98 (d, J=9.2 Hz, 8H), 1.66 (s, 2H), 1.37 (d, J=11.7 Hz, 2H). LCMS (ESI) calcd, for C₄₂H₄₉N₁₂O₅⁺ [M+H]⁺: 801.39, found, 801.4.

### Example 201: Preparation of 7-cyclopentyl-2-((5-(4-((3-(2-(2,4-dioxotetrahydropyrimidin-1(2H)-yl)-1,3-dioxoisoindolin-5-yl)-3,8-diazabicyclo[3.2.1]octan-8-yl)methyl)piperidin-1-yl)pyridin-2-yl)amino)-N,N-dimethyl-7H-pyrrolo[2,3-d]pyrimidine-6-carboxamide (GT-05812)

With reference to the method of Scheme T-3, the target compound (GT-05812) was obtained (yellow solid, 25 mg, yield 35.40%). ¹H NMR (400 MHz, DMSO-d₆) δ 11.58 (s, 1H), 11.24 (s, 1H), 10.86 (s, 1H), 9.01 (d, J=3.3 Hz, 1H), 8.19 (dd, J=9.6, 2.6 Hz, 1H), 7.98 (s, 1H), 7.81 (d, J=8.5 Hz, 1H), 7.62 (d, J=9.5 Hz, 1H), 7.45 (d, J=1.9 Hz, 1H), 7.34-7.25 (m, 1H), 6.84 (s, 1H), 4.81 (d, J=8.7 Hz, 1H), 4.22 (s, 2H), 4.02 (d, J=14.2 Hz, 2H), 3.88 (d, J=12.0 Hz, 2H), 3.80 (d, J=6.9 Hz, 2H), 3.74 (s, 2H), 3.35 (s, 1H), 3.11-2.98 (m, 8H), 2.85 (dd, J=14.9, 8.1 Hz, 4H), 2.27 (d, J=34.5 Hz, 5H), 2.11-1.92 (m, 8H), 1.73-1.61 (m, 2H), 1.47 (d, J=9.5 Hz, 2H). LCMS (ESI) calcd, for C₄₃H₅₁N₁₂O₅⁺ [M+H]⁺: 815.41, found, 815.5.

### Example 202: Preparation of 7-cyclopentyl-2-((5-(4-((5-(2-(2,4-dioxotetrahydropyrimidin-1(2H)-yl)-1,3-dioxoisoindolin-5-yl)-2,5-diazabicyclo[2.2.2]octan-2-yl)methyl)piperidin-1-yl)pyridin-2-yl)amino)-N,N-dimethyl-7H-pyrrolo[2,3-d]pyrimidine-6-carboxamide (GT-05831)

With reference to the method of Scheme T-3, the target compound (GT-05831) was obtained (yellow solid, 30 mg, yield 33.98%). ¹H NMR (400 MHz, DMSO-d₆) δ 11.57 (s, 1H), 10.97 (s, 1H), 10.85 (s, 1H), 9.01 (s, 1H), 8.18 (d, J=9.7 Hz, 1H), 7.97 (s, 1H), 7.78 (s, 1H), 7.64-7.51 (m, 1H), 7.24 (d, J=10.7 Hz, 1H), 7.07 (d, J=9.2 Hz, 1H), 6.84 (s, 1H), 4.80 (d, J=8.8 Hz, 1H), 4.61 (s, 1H), 4.34-4.10 (m, 1H), 3.99 (s, 1H), 3.87 (s, 1H), 3.79 (t, J=7.0 Hz, 2H), 3.69 (s, 5H), 3.23 (s, 2H), 3.06 (s, 6H), 2.83 (dd, J=16.0, 8.8 Hz, 4H), 2.32 (d, J=11.9 Hz, 2H), 2.02 (dd, J=30.0, 9.9 Hz, 10H), 1.72-1.59 (m, 2H), 1.43 (s, 2H). LCMS (ESI) calcd. for C₄₃H₅₁N₁₂O₅⁺ [M+H]⁺: 815.41, found, 815.4.

### Example 203: Preparation of 7-cyclopentyl-2-((5-(4-((2-(2,4-dioxotetrahydropyrimidin-1(2H)-yl)-1-oxoisoindolin-5-yl)methyl)piperazin-1-yl)pyridin-2-yl)amino)-N,N-dimethyl-7H-pyrrolo[2,3-d]pyrimidine-6-carboxamide (GT-06335)

With reference to the method of Step 2 in Scheme T-1, the target compound (GT-06335) was obtained (yellow solid, 35 mg, yield 54.96%). ¹H NMR (400 MHz, DMSO-d₆) δ 11.67 (s, 2H), 10.68 (d, J=12.7 Hz, 1H), 9.04 (d, J=21.5 Hz, 1H), 8.07 (d, J=9.0 Hz, 1H), 7.98 (s, 2H), 7.87 (s, 2H), 7.64-7.54 (m, 1H), 6.82 (d, J=18.6 Hz, 1H), 4.87-4.71 (m, 2H), 4.58 (d, J=12.4 Hz, 3H), 3.80 (d, J=6.4 Hz, 6H), 3.31 (s, 4H), 3.04 (s, 6H), 2.81 (dd, J=44.6, 12.0 Hz, 2H), 2.28 (s, 2H), 1.96 (dd, J=24.9, 18.2 Hz, 4H), 1.63 (d, J=5.4 Hz, 2H). LCMS (ESI) calcd, for C₃₆H₄₂N₁₁O₄⁺ [M+H]⁺: 692.34, found, 692.4.

### Example 204: Preparation of 7-cyclopentyl-2-((5-(4-((2-(2,4-dioxotetrahydropyrimidin-1(2H)-yl)-1,3-dioxoisoindolin-5-yl)methyl)piperazin-1-yl)pyridin-2-yl)amino)-N,N-dimethyl-7H-pyrrolo[2,3-d]pyrimidine-6-carboxamide (GT-06336)

With reference to the method of Step 2 in Scheme T-1, the target compound (GT-06336) was obtained (yellow solid, 38 mg, yield 58.49%). ¹H NMR (400 MHz, DMSO-d₆) δ 11.59 (s, 1H), 10.94 (s, 1H), 9.02 (s, 1H), 8.38 (s, 1H), 8.25 (dd, J=7.8, 1.1 Hz, 1H), 8.12 (d, J=7.7 Hz, 1H), 8.10-8.06 (m, 1H), 7.97 (d, J=2.9 Hz, 2H), 7.60 (d, J=9.4 Hz, 1H), 6.85 (s, 1H), 4.79 (dd, J=17.5, 8.7 Hz, 1H), 4.65 (s, 1H), 4.53 (d, J=15.1 Hz, 1H), 3.84 (d, J=6.8 Hz, 2H), 3.66 (s, 4H), 3.33-3.29 (m, 3H), 3.06 (s, 6H), 2.87 (t, J=6.8 Hz, 2H), 2.79-2.73 (m, 1H), 2.30 (dd, J=12.0, 8.5 Hz, 2H), 2.07-1.94 (m, 4H), 1.70-1.59 (m, 2H). LCMS (ESI) calcd. for C₃₆H₄₀N₁₁O₅⁺ [M+H]⁺: 706.32, found, 706.3.

### Example 205: Preparation of 7-cyclopentyl-2-((5-(4-((1-(2-(2,4-dioxotetrahydropyrimidin-1(2H)-yl)-1,3-dioxoisoindolin-5-yl)piperidin-4-yl)methyl)piperazin-1-yl)pyridin-2-yl)amino)-N,N-dimethyl-7H-pyrrolo[2,3-d]pyrimidine-6-carboxamide (GT-06337)

With reference to the method of Scheme T-3, the target compound (GT-06337) was obtained (yellow solid, 10 mg, yield 11.02%). ¹H NMR (400 MHz, DMSO-d₆) δ 11.64 (s, 1H), 10.84 (s, 1H), 10.38 (d, J=37.0 Hz, 1H), 9.03 (s, 1H), 8.12 (d, J=11.2 Hz, 1H), 7.99 (s, 1H), 7.75 (d, J=8.9 Hz, 1H), 7.61 (d, J=9.4 Hz, 1H), 7.45-7.27 (m, 1H), 7.03 (dd, J=28.0, 17.5 Hz, 1H), 6.85 (s, 1H), 4.86-4.75 (m, 1H), 4.10 (s, 1H), 3.99-3.76 (m, 10H), 3.22-3.16 (m, 4H), 3.05 (s, 6H), 2.92 (d, J=10.9 Hz, 2H), 2.75 (dd, J=11.4, 6.8 Hz, 2H), 2.30 (s, 3H), 1.99 (d, J=13.0 Hz, 6H), 1.65 (d, J=5.5 Hz, 2H), 1.31 (d, J=9.5 Hz, 2H). LCMS (ESI) calcd. for C₄₁H₄₉N₁₂O₅⁺ [M+H]⁺: 789.39, found, 789.4.

### Example 206: Preparation of 7-cyclopentyl-2-((5-(4-((1-(2-(2,4-dioxotetrahydropyrimidin-1(2H)-yl)-1,3-dioxoisoindolin-4-yl)piperidin-4-yl)methyl)piperazin-1-yl)pyridin-2-yl)amino)-N,N-dimethyl-7H-pyrrolo[2,3-d]pyrimidine-6-carboxamide (GT-06338)

With reference to the method of Scheme T-3, the target compound (GT-06338) was obtained (yellow solid, 18 mg, yield 19.83%). ¹H NMR (400 MHz, DMSO-d₆) δ 11.51 (s, 1H), 10.86 (s, 1H), 9.46 (s, 1H), 9.01 (d, J=1.4 Hz, 1H), 8.15-7.95 (m, 2H), 7.74 (d, J=7.4 Hz, 1H), 7.64 (dd, J=9.3, 5.9 Hz, 1H), 7.46-7.35 (m, 1H), 6.83 (s, 1H), 4.86-4.69 (m, 1H), 3.90-3.64 (m, 10H), 3.15 (s, 8H), 3.05 (s, 6H), 2.93 (s, 1H), 2.84-2.80 (m, 1H), 2.30 (s, 2H), 2.11-1.92 (m, 6H), 1.64 (d, J=4.3 Hz, 2H), 1.48 (s, 1H). LCMS (ESI) calcd. for C₄₁H₄₉N₁₂O₅⁺ [M+H]⁺: 789.39, found, 789.4.

### Example 207: Preparation of 7-cyclopentyl-2-((5-(4-(((2-(2,4-dioxotetrahydropyrimidin-1(2H)-yl)-1-oxoisoindolin-5-yl)methyl)(methyl)amino)piperidin-1-yl)pyridin-2-yl)amino)-N,N-dimethyl-7H-pyrrolo[2,3-d]pyrimidine-6-carboxamide (GT-06487)

With reference to the method of Step 2 in Scheme T-1, the target compound (GT-06487) was obtained (yellow solid, 18 mg, yield 23.14%). ¹H NMR (400 MHz, DMSO-d₆) δ 11.52 (d, J=85.7 Hz, 2H), 10.68 (d, J=16.5 Hz, 1H), 9.02 (s, 1H), 8.17 (dd, J=9.6, 2.7 Hz, 1H), 7.97 (dd, J=14.1, 11.6 Hz, 2H), 7.88 (dd, J=15.0, 8.0 Hz, 2H), 7.62 (d, J=9.5 Hz, 1H), 6.84 (s, 1H), 4.89-4.73 (m, 2H), 4.66-4.57 (m, 2H), 4.38 (dd, J=12.7, 7.0 Hz, 1H), 3.81 (d, J=6.8 Hz, 4H), 3.42 (s, 2H), 3.06 (s, 6H), 2.82 (dd, J=21.8, 9.4 Hz, 4H), 2.62 (d, J=4.2 Hz, 3H), 2.35 (dd, J=36.2, 7.8 Hz, 4H), 1.98 (dd, J=18.5, 11.7 Hz, 5H), 1.71-1.61 (m, 2H). LCMS (ESI) calcd. for C₃₈H₄₆N₁₁O₄⁺ [M+H]⁺: 720.37, found, 720.4.

### Example 208: Preparation of 7-cyclopentyl-2-((5-(4-(((2-(2,4-dioxotetrahydropyrimidin-1(2H)-yl)-1,3-dioxoisoindolin-5-yl)methyl)(methyl)amino)piperidin-1-yl)pyridin-2-yl)amino)-N,N-dimethyl-7H-pyrrolo[2,3-d]pyrimidine-6-carboxamide (GT-06488)

With reference to the method of Step 2 in Scheme T-1, the target compound (GT-06488) was obtained (yellow solid, 30 mg, yield 37.82%). ¹H NMR (400 MHz, DMSO-d₆) δ 11.60 (s, 1H), 10.94 (s, 1H), 10.73-10.33 (m, 1H), 9.02 (s, 1H), 8.37 (dd, J=47.0, 6.9 Hz, 1H), 8.17 (d, J=9.1 Hz, 1H), 8.06 (dd, J=30.9, 8.2 Hz, 1H), 7.97 (s, 1H), 7.62 (d, J=9.5 Hz, 1H), 6.84 (s, 1H), 4.82 (p, J=8.7 Hz, 1H), 4.66 (d, J=31.9 Hz, 1H), 4.41 (d, J=50.3 Hz, 1H), 3.87 (d, J=11.1 Hz, 2H), 3.70-3.65 (m, 3H), 3.07 (s, 6H), 2.86 (d, J=6.8 Hz, 4H), 2.66-2.59 (m, 3H), 2.40-2.27 (m, 4H), 1.99 (d, J=6.7 Hz, 6H), 1.71-1.53 (m, 2H). LCMS (ESI) calcd. for C₃₈H₄₄N₁₁O₅⁺ [M+H]⁺: 734.35, found, 734.4.

### Example 209: Preparation of 7-cyclopentyl-2-((5-(4-(((1-(2-(2,4-dioxotetrahydropyrimidin-1(2H)-yl)-1,3-dioxoisoindolin-5-yl)piperidin-4-yl)methyl)(methyl)amino)piperidin-1-yl)pyridin-2-yl)amino)-N,N-dimethyl-7H-pyrrolo[2,3-d]pyrimidine-6-carboxamide (GT-06489)

With reference to the method of Scheme T-3, the target compound (GT-06489) was obtained (yellow solid, 5 mg, yield 5.66%). ¹H NMR (400 MHz, DMSO-d₆) δ 11.60 (s, 1H), 10.85 (s, 1H), 10.38 (dd, J=34.1, 7.9 Hz, 2H), 9.02 (s, 1H), 8.15 (d, J=9.6 Hz, 1H), 7.95 (s, 1H), 7.77-7.54 (m, 2H), 7.45 (d, J=33.7 Hz, 1H), 7.38-7.23 (m, 1H), 6.84 (s, 1H), 4.81 (dd, J=17.3, 8.6 Hz, 1H), 4.14 (d, J=13.4 Hz, 2H), 3.85 (d, J=11.5 Hz, 4H), 3.66 (s, 2H), 3.41-3.39 (m, 2H), 3.16 (s, 2H), 3.06 (s, 7H), 2.80 (dd, J=21.2, 5.7 Hz, 5H), 2.36-2.26 (m, 3H), 2.18 (s, 2H), 2.06-1.95 (m, 4H), 1.85 (s, 2H), 1.66 (d, J=5.5 Hz, 2H), 1.29 (dd, J=12.7, 6.7 Hz, 2H). LCMS (ESI) calcd, for C₄₃H₅₃N₁₂O₅⁺ [M+H]⁺: 817.43, found, 817.5.

### Example 210: Preparation of 7-cyclopentyl-2-((5-(4-(4-((2-(2,4-dioxotetrahydropyrimidin-1(2H)-yl)-1-oxoisoindolin-5-yl)methyl)piperazin-1-yl)piperidin-1-yl)pyridin-2-yl)amino)-N,N-dimethyl-7H-pyrrolo[2,3-d]pyrimidine-6-carboxamide (GT-06497)

With reference to the method of Step 2 in Scheme T-1, the target compound (GT-06497) was obtained (yellow solid, 18 mg, yield 30.06%). ¹H NMR (400 MHz, DMSO-d₆) δ 12.22 (s, 1H), 11.57 (s, 1H), 10.69 (s, 1H), 9.00 (s, 1H), 8.14 (dd, J=9.6, 2.6 Hz, 1H), 7.98-7.89 (m, 2H), 7.84 (s, 2H), 7.58 (d, J=9.5 Hz, 1H), 6.83 (s, 1H), 4.87-4.71 (m, 2H), 4.55 (dd, J=38.9, 22.0 Hz, 3H), 3.79 (s, 5H), 3.68 (s, 3H), 3.56 (s, 6H), 3.05 (s, 6H), 2.87-2.74 (m, 4H), 2.34-2.20 (m, 4H), 2.00 (dd, J=19.4, 7.7 Hz, 4H), 1.86 (d, J=9.0 Hz, 1H), 1.69-1.60 (m, 2H). LCMS (ESI) calcd, for C₄₁H₅₁N₁₂O₄⁺ [M+H]⁺: 775.42, found, 775.42.

### Example 211: Preparation of 7-cyclopentyl-2-((5-(4-(4-((2-(2,4-dioxotetrahydropyrimidin-1(2H)-yl)-1,3-dioxoisoindolin-5-yl)methyl)piperazin-1-yl)piperidin-1-yl)pyridin-2-yl)amino)-N,N-dimethyl-7H-pyrrolo[2,3-d]pyrimidine-6-carboxamide (GT-06498)

With reference to the method of Step 2 in Scheme T-1, the target compound (GT-06498) was obtained (yellow solid, 18 mg, yield 29.53%). ¹H NMR (400 MHz, DMSO-d₆) δ 12.35 (s, 1H), 11.62 (s, 1H), 10.93 (s, 1H), 9.01 (s, 1H), 8.16 (d, J=9.4 Hz, 2H), 7.95 (s, 2H), 7.60 (d, J=9.5 Hz, 1H), 6.84 (s, 1H), 4.81 (s, 1H), 4.63-4.51 (m, 2H), 3.90-3.82 (m, 6H), 3.70 (s, 3H), 3.39-3.30 (m, 4H), 3.06 (s, 6H), 2.89-2.75 (m, 4H), 2.27 (dd, J=18.0, 9.6 Hz, 4H), 2.08-1.95 (m, 4H), 1.88 (d, J=7.2 Hz, 2H), 1.65 (d, J=5.9 Hz, 2H). LCMS (ESI) calcd, for C₄₁H₄₉N₁₂O₅⁺ [M+H]⁺: 789.39, found, 789.5.

### Example 212: Preparation of 1-(5-((4-(6-((6-acetyl-8-cyclopentyl-5-methyl-7-oxo-7,8-dihydropyrido[2,3-d]pyrimidin-2-yl)amino)pyridin-3-yl)piperazin-1-yl)methyl)-1-oxoisoindolin-2-yl)dihydropyrimidine-2,4 (1H,3H)-dione (GT-06573)

With reference to the method of Step 2 in Scheme T-1, the target compound (GT-06573) was obtained (yellow solid, 30 mg, yield 47.63%). ¹H NMR (400 MHz, DMSO-d₆) δ 11.54 (s, 1H), 10.78 (d, J=54.1 Hz, 1H), 10.70 (s, 1H), 8.99 (s, 1H), 8.07 (d, J=2.6 Hz, 1H), 7.94 (s, 1H), 7.91-7.78 (m, 4H), 5.84 (s, 1H), 4.79 (d, J=5.2 Hz, 1H), 4.58 (d, J=25.7 Hz, 3H), 3.89-3.78 (m, 4H), 3.25 (s, 6H), 2.82 (d, J=30.2 Hz, 2H), 2.43 (s, 3H), 2.34 (d, J=10.8 Hz, 3H), 2.28-2.14 (m, 2H), 1.92 (s, 2H), 1.86-1.72 (m, 2H), 1.66-1.54 (m, 2H). LCMS (ESI) calcd, for C₃₇H₄₁N₁₀O₅⁺ [M+H]⁺: 705.33, found, 705.4.

### Example 213: Preparation of 5-((4-(6-((6-acetyl-8-cyclopentyl-5-methyl-7-oxo-7,8-dihydropyrido[2,3-d]pyrimidin-2-yl)amino)pyridin-3-yl)piperazin-1-yl)methyl)-2-(2,4-dioxotetrahydropyrimidin-1(2H)-yl)isoindoline-1,3-dione (GT-06574)

With reference to the method of Step 2 in Scheme T-1, the target compound (GT-06574) was obtained (yellow solid, 24 mg, yield 37.36%). ¹H NMR (400 MHz, DMSO-d₆) δ 11.65-11.26 (m, 1H), 10.93 (s, 2H), 10.55 (dd, J=55.0, 3.6 Hz, 1H), 9.00 (s, 1H), 8.26 (d, J=57.7 Hz, 1H), 8.12 (d, J=7.7 Hz, 1H), 8.07 (s, 1H), 7.83 (s, 2H), 5.83 (s, 1H), 4.63 (s, 2H), 3.82 (t, J=6.8 Hz, 3H), 3.67-3.58 (m, 4H), 3.28-3.15 (m, 6H), 2.83 (dd, J=27.5, 20.7 Hz, 2H), 2.43 (s, 3H), 2.33 (s, 3H), 2.22 (d, J=3.2 Hz, 2H), 1.92 (s, 2H), 1.83-1.73 (m, 2H), 1.58 (d, J=5.1 Hz, 2H). LCMS (ESI) calcd, for C₃₇H₃₉N₁₀O₆⁺ [M+H]⁺: 719.31, found, 719.3.

### Example 214: Preparation of 5-((4-(1-(6-((6-acetyl-8-cyclopentyl-5-methyl-7-oxo-7,8-dihydropyrido[2,3-d]pyrimidin-2-yl)amino)pyridin-3-yl)piperidin-4-yl)piperazin-1-yl)methyl)-2-(2,4-dioxotetrahydropyrimidin-1(2H)-yl)isoindoline-1,3-dione (GT-06583)

With reference to the method of Step 2 in Scheme T-1, the target compound (GT-06583) was obtained (yellow solid, 25 mg, yield 41.36%). ¹H NMR (400 MHz, DMSO-d₆) δ 12.08-11.44 (m, 1H), 11.11 (s, 1H), 10.95-10.55 (m, 1H), 9.01 (s, 1H), 8.18 (d, J=50.8 Hz, 1H), 8.06 (d, J=7.8 Hz, 2H), 8.01-7.85 (m, 2H), 7.80 (d, J=9.4 Hz, 1H), 5.84 (p, J=8.8 Hz, 1H), 4.36 (s, 2H), 3.87 (dd, J=28.3, 9.3 Hz, 4H), 3.68-3.63 (m, 5H), 3.41-3.38 (m, 4H), 2.86-2.73 (m, 4H), 2.43 (d, J=7.4 Hz, 3H), 2.34 (s, 3H), 2.23 (s, 4H), 2.00-1.78 (m, 6H), 1.64-1.55 (m, 2H). LCMS (ESI) calcd, for C₄₂H₄₈N₁₁O₆⁺ [M+H]⁺: 802.38, found, 802.4.

### Example 215: Preparation of 1-(5-(((1-(6-((6-acetyl-8-cyclopentyl-5-methyl-7-oxo-7,8-dihydropyrido[2,3-d]pyrimidin-2-yl)amino)pyridin-3-yl)piperidin-4-yl)(methyl)amino)methyl)-1-oxoisoindolin-2-yl)dihydropyrimidine-2,4 (1H,3H)-dione (GT-06586)

With reference to the method of Step 2 in Scheme T-1, the target compound (GT-06586) was obtained (yellow solid, 20 mg, yield 28.84%). ¹H NMR (400 MHz, DMSO-d₆) δ 11.25 (d, J=70.7 Hz, 2H), 10.69 (s, 1H), 9.02 (s, 1H), 8.08 (d, J=2.1 Hz, 1H), 7.99 (s, 2H), 7.92-7.80 (m, 3H), 5.84 (s, 1H), 4.82 (d, J=19.7 Hz, 1H), 4.62 (s, 2H), 4.45-4.35 (m, 1H), 3.91 (s, 2H), 3.80 (d, J=6.7 Hz, 2H), 3.47-3.38 (m, 2H), 2.83 (dd, J=26.0, 12.0 Hz, 4H), 2.62 (d, J=4.2 Hz, 3H), 2.44 (s, 3H), 2.35 (s, 3H), 2.29-2.16 (m, 3H), 1.95 (s, 4H), 1.81 (d, J=4.7 Hz, 2H), 1.60 (d, J=5.0 Hz, 2H). LCMS (ESI) calcd, for C₃₉H₄₅N₁₀O₅⁺ [M+H]⁺: 733.36, found, 733.4.

### Example 216: Preparation of 5-(((1-(6-((6-acetyl-8-cyclopentyl-5-methyl-7-oxo-7,8-dihydropyrido[2,3-d]pyrimidin-2-yl)amino)pyridin-3-yl)piperidin-4-yl)(methyl)amino)methyl)-2-(2,4-dioxotetrahydropyrimidin-1(2H)-yl)isoindoline-1,3-dione (GT-06587)

With reference to the method of Step 2 in Scheme T-1, the target compound (GT-06587) was obtained (yellow solid, 26 mg, yield 36.80%). ¹H NMR (400 MHz, DMSO-d₆) δ 11.46-11.06 (m, 2H), 10.94 (s, 1H), 9.02 (s, 1H), 8.40 (d, J=4.2 Hz, 1H), 8.28 (d, J=7.7 Hz, 1H), 8.14-8.05 (m, 2H), 7.99 (d, J=7.7 Hz, 1H), 7.82 (d, J=9.4 Hz, 1H), 5.97-5.61 (m, 1H), 4.71 (t, J=19.6 Hz, 1H), 4.53-4.34 (m, 1H), 3.94 (s, 2H), 3.83 (t, J=6.7 Hz, 2H), 3.66 (s, 2H), 3.38-3.34 (m, 3H), 2.63 (s, 3H), 2.45 (s, 3H), 2.35 (d, J=7.7 Hz, 4H), 2.30-2.18 (m, 3H), 1.95 (s, 4H), 1.84-1.76 (m, 2H), 1.67-1.57 (m, 2H). LCMS (ESI) calcd, for C₃₉H₄₃N₁₀O₆⁺ [M+H]⁺: 747.34, found, 747.4.

### Example 217: Preparation of 2-(2,4-dioxotetrahydropyrimidin-1(2H)-yl)-5-((4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)methyl)isoindoline-1,3-dione (GT-06661)

With reference to the method of Step 2 in Scheme T-1, the target compound (GT-06661) was obtained (yellow solid, 24 mg, yield 38.30%). ¹H NMR (400 MHz, DMSO-d₆) δ 11.37 (s, 1H), 10.62 (dd, J=119.4, 62.6 Hz, 2H), 8.86 (d, J=3.3 Hz, 1H), 8.60 (s, 1H), 8.40 (s, 1H), 8.38-8.28 (m, 1H), 8.12 (dd, J=27.9, 8.2 Hz, 2H), 7.88 (d, J=12.0 Hz, 2H), 5.02-4.93 (m, 1H), 4.47 (s, 4H), 3.82 (d, J=6.8 Hz, 1H), 3.66 (t, J=6.7 Hz, 2H), 3.47 (dd, J=20.2, 13.2 Hz, 7H), 2.90-2.73 (m, 5H), 1.68 (d, J=6.9 Hz, 6H). LCMS (ESI) calcd. for C₃₈H₃₈F₂N₁₁O₄⁺ [M+H]⁺: 750.31, found, 750.3.

### Example 218: Preparation of 2-(2,4-dioxotetrahydropyrimidin-1(2H)-yl)-5-(4-((4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)methyl)piperidin-1-yl)isoindoline-1,3-dione (GT-06662)

With reference to the method of Scheme T-3, the target compound (GT-06662) was obtained (yellow solid, 13 mg, yield 14.94%). ¹H NMR (400 MHz, DMSO-d₆) δ 11.43 (s, 1H), 10.84 (s, 1H), 8.88 (d, J=3.1 Hz, 1H), 8.63 (s, 1H), 8.42 (s, 1H), 8.35 (d, J=9.0 Hz, 1H), 8.16 (d, J=8.8 Hz, 1H), 7.91 (d, J=11.5 Hz, 1H), 7.70 (dd, J=16.1, 6.5 Hz, 1H), 7.49-7.30 (m, 2H), 5.03-4.95 (m, 1H), 4.49 (s, 2H), 3.80 (s, 4H), 3.65 (d, J=5.5 Hz, 7H), 3.04 (dd, J=34.1, 22.3 Hz, 4H), 2.82 (s, 5H), 2.75-2.67 (m, 1H), 2.17 (s, 1H), 2.00 (d, J=9.8 Hz, 2H), 1.69 (d, J=6.8 Hz, 6H), 1.29 (dd, J=14.1, 6.5 Hz, 2H). LCMS (ESI) calcd, for C₄₃H₄₇F₂N₁₂O₄⁺ [M+H]⁺: 833.38, found, 833.4.

### Example 219: Preparation of 2-(2,4-dioxotetrahydropyrimidin-1(2H)-yl)-4-(4-((4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)methyl)piperidin-1-yl)isoindoline-1,3-dione (GT-06663)

With reference to the method of Scheme T-3, the target compound (GT-06663) was obtained (yellow solid, 13 mg, yield 14.94%). ¹H NMR (400 MHz, DMSO-d₆) δ 11.61 (s, 2H), 10.84 (s, 1H), 8.89 (d, J=3.2 Hz, 1H), 8.65 (s, 1H), 8.41 (m, 2H), 8.14 (m, 1H), 7.93 (d, J=11.5 Hz, 1H), 7.73 (m, 1H), 7.39 (t, J=8.2 Hz, 2H), 4.98 (m, 1H), 4.50 (s, 3H), 3.77 (ddd, J=36.4, 18.3, 11.5 Hz, 10H), 3.52 (d, J=36.1 Hz, 2H), 3.15 (d, J=10.0 Hz, 2H), 2.87 (m, 7H), 2.06 (s, 3H), 1.69 (d, J=6.8 Hz, 6H), 1.42 (dd, J=33.6, 24.2 Hz, 1H). LCMS (ESI) calcd. for C₄₃H₄₇F₂N₁₂O₄⁺ [M+H]⁺: 833.38, found, 833.4.

### Example 220: Preparation of 1-(5-(((1-(6-((5-fluoro-4-(4-fluoro-1-isopropyl-2-methyl-1H-benzo[d]imidazol-6-yl)pyrimidin-2-yl)amino)pyridin-3-yl)piperidin-4-yl)(methyl)amino)methyl)-1-oxoisoindolin-2-yl)dihydropyrimidine-2,4 (1H,3H)-dione (GT-06837)

With reference to the method of Step 2 in Scheme T-1, the target compound (GT-06837) was obtained (yellow solid, 24 mg, yield 45.05%). ¹H NMR (400 MHz, DMSO-d₆) δ 11.77 (s, 1H), 11.49 (s, 1H), 10.69 (s, 1H), 8.86 (d, J=3.3 Hz, 1H), 8.30 (s, 1H), 8.22 (d, J=8.9 Hz, 1H), 7.99 (d, J=13.0 Hz, 2H), 7.91-7.77 (m, 4H), 4.92 (dt, J=13.0, 6.4 Hz, 1H), 4.82 (dd, J=16.9, 8.4 Hz, 1H), 4.60 (dd, J=30.6, 13.1 Hz, 2H), 4.37 (dd, J=12.9, 6.9 Hz, 1H), 3.87 (s, 4H), 3.43 (d, J=7.0 Hz, 2H), 2.84 (d, J=8.9 Hz, 2H), 2.75 (d, J=6.5 Hz, 4H), 2.61 (d, J=4.3 Hz, 3H), 2.40 (d, J=12.0 Hz, 1H), 2.30 (d, J=11.4 Hz, 1H), 1.97 (dd, J=20.9, 9.4 Hz, 2H), 1.64 (d, J=6.9 Hz, 6H). LCMS (ESI) calcd, for C₃₉H₄₂F₂N₁₁O₃⁺ [M+H]⁺: 750.34, found, 750.3.

### Example 221: Preparation of 2-(2,4-dioxotetrahydropyrimidin-1(2H)-yl)-5-(((1-(6-((5-fluoro-4-(4-fluoro-1-isopropyl-2-methyl-1H-benzo[d]imidazol-6-yl)pyrimidin-2-yl)amino)pyridin-3-yl)piperidin-4-yl)(methyl)amino)methyl)isoindoline-1,3-dione (GT-06732)

With reference to the method of Step 2 in Scheme T-1, the target compound (GT-06732) was obtained (yellow solid, 16 mg, yield 22.93%). ¹H NMR (400 MHz, DMSO-d₆) δ 11.62 (s, 1H), 10.93 (s, 1H), 10.54 (d, J=59.4 Hz, 1H), 8.85 (d, J=3.3 Hz, 1H), 8.28 (s, 2H), 8.02 (t, J=47.3 Hz, 4H), 7.79 (dd, J=20.2, 10.7 Hz, 2H), 4.90 (s, 1H), 4.66 (d, J=32.1 Hz, 1H), 4.41 (d, J=54.3 Hz, 1H), 3.87 (d, J=20.2 Hz, 3H), 3.69 (s, 2H), 2.86 (t, J=6.6 Hz, 3H), 2.76-2.70 (m, 4H), 2.62 (s, 3H), 2.33 (s, 2H), 1.95 (s, 2H), 1.64 (d, J=6.9 Hz, 6H). LCMS (ESI) calcd, for C₃₉H₄₀F₂N₁₁O₄⁺ [M+H]⁺: 764.32, found, 764.3.

### Example 222: Preparation of 2-(2,4-dioxotetrahydropyrimidin-1(2H)-yl)-5-(4-(((1-(6-((5-fluoro-4-(4-fluoro-1-isopropyl-2-methyl-1H-benzo[d]imidazol-6-yl)pyrimidin-2-yl)amino)pyridin-3-yl)piperidin-4-yl)(methyl)amino)methyl)piperidin-1-yl)isoindoline-1,3-dione (GT-06733)

With reference to the method of Step 2 in Scheme T-1, the target compound was obtained (GT-06733) (yellow solid, 21 mg, yield 24.43%). ¹H NMR (400 MHz, DMSO-d₆) δ 11.54 (s, 1H), 10.84 (s, 2H), 10.24 (s, 1H), 8.84 (d, J=3.4 Hz, 1H), 8.27 (s, 1H), 8.13 (d, J=7.4 Hz, 1H), 7.95 (s, 1H), 7.80-7.70 (m, 4H), 7.45-7.39 (m, 2H), 7.35-7.28 (m, 2H), 4.90 (s, 1H), 4.14 (d, J=12.6 Hz, 2H), 3.97 (d, J=13.5 Hz, 1H), 3.88 (s, 1H), 3.82-3.77 (m, 4H), 3.18 (s, 2H), 3.04 (d, J=12.0 Hz, 2H), 2.83 (t, J=6.6 Hz, 5H), 2.78 (d, J=4.8 Hz, 2H), 2.72 (d, J=10.4 Hz, 4H), 2.17 (d, J=9.8 Hz, 3H), 1.97-1.82 (m, 4H), 1.64 (d, J=6.9 Hz, 6H), 1.26 (d, J=19.4 Hz, 2H). LCMS (ESI) calcd, for C₄₄H₄₉F₂N₁₂O₄⁺ [M+H]⁺: 847.40, found, 847.4.

### Example 223: Preparation of 2-(2,4-dioxotetrahydropyrimidin-1(2H)-yl)-4-(4-(((1-(6-((5-fluoro-4-(4-fluoro-1-isopropyl-2-methyl-1H-benzo[d]imidazol-6-yl)pyrimidin-2-yl)amino)pyridin-3-yl)piperidin-4-yl)(methyl)amino)methyl)piperidin-1-yl)isoindoline-1,3-dione (GT-06734)

With reference to the method of Scheme T-3, the target compound (GT-06734) was obtained (yellow solid, 26 mg, yield 30.24%). ¹H NMR (400 MHz, DMSO-d₆) δ 11.59 (s, 1H), 10.85 (s, 2H), 10.36 (s, 1H), 8.84 (d, J=3.4 Hz, 1H), 8.27 (s, 1H), 8.16 (d, J=8.0 Hz, 1H), 7.95 (s, 1H), 7.75 (dt, J=10.9, 5.5 Hz, 3H), 7.46-7.35 (m, 3H), 4.93-4.83 (m, 1H), 3.90-3.78 (m, 6H), 3.21 (s, 4H), 2.95-2.79 (m, 9H), 2.72 (d, J=10.4 Hz, 3H), 2.31-2.21 (m, 2H), 2.05 (s, 1H), 1.88 (s, 3H), 1.63 (t, J=6.9 Hz, 6H), 1.47 (s, 2H). LCMS (ESI) calcd. for C₄₄H₄₉F₂N₁₂O₄⁺ [M+H]⁺: 847.40, found, 847.4.

### Example 224: Preparation of 1-(5-((4-(6-((5-fluoro-4-(4-fluoro-1-isopropyl-2-methyl-1H-benzo[d]imidazol-6-yl)pyrimidin-2-yl)amino)pyridin-3-yl)piperazin-1-yl)methyl)-1-oxoisoindolin-2-yl)dihydropyrimidine-2,4 (1H,3H)-dione (GT-06672)

With reference to the method of Step 2 in Scheme T-1, the target compound (GT-06672) was obtained (yellow solid, 35 mg, yield 50.06%). ¹H NMR (400 MHz, DMSO-d₆) δ 11.97 (s, 1H), 11.70 (s, 1H), 10.70 (s, 1H), 8.86 (d, J=3.3 Hz, 1H), 8.32 (s, 1H), 8.13 (d, J=9.1 Hz, 1H), 7.98 (d, J=5.2 Hz, 2H), 7.85 (dt, J=15.7, 9.9 Hz, 4H), 4.98-4.77 (m, 2H), 4.59 (d, J=19.3 Hz, 3H), 3.83 (s, 3H), 3.48-3.43 (m, 3H), 3.31 (d, J=30.3 Hz, 4H), 2.93-2.84 (m, 1H), 2.78 (d, J=10.9 Hz, 4H), 1.65 (d, J=6.9 Hz, 6H). LCMS (ESI) calcd. for C₃₇H₃₈F₂N₁₁O₃⁺ [M+H]⁺: 722.31, found, 722.3.

### Example 225: Preparation of 2-(2,4-dioxotetrahydropyrimidin-1(2H)-yl)-5-((4-(6-((5-fluoro-4-(4-fluoro-1-isopropyl-2-methyl-1H-benzo[d]imidazol-6-yl)pyrimidin-2-yl)amino)pyridin-3-yl)piperazin-1-yl)methyl)isoindoline-1,3-dione (GT-06673)

With reference to the method of Step 2 in Scheme T-1, the target compound (GT-06673) was obtained (yellow solid, 31 mg, yield 43.49%). ¹H NMR (400 MHz, DMSO-d₆) δ 12.30 (s, 1H), 11.83 (s, 1H), 10.93 (s, 1H), 8.87 (d, J=3.2 Hz, 1H), 8.40 (s, 1H), 8.34 (s, 1H), 8.27 (d, J=7.8 Hz, 1H), 8.17 (d, J=7.4 Hz, 1H), 8.11 (d, J=7.7 Hz, 1H), 7.99-7.93 (m, 1H), 7.85 (dd, J=12.8, 10.8 Hz, 2H), 4.94 (dt, J=13.9, 6.9 Hz, 1H), 4.68 (d, J=13.2 Hz, 2H), 4.53 (d, J=16.8 Hz, 1H), 3.83 (t, J=6.7 Hz, 3H), 3.48-3.25 (m, 6H), 2.85 (dd, J=13.5, 6.7 Hz, 2H), 2.79 (s, 3H), 1.65 (d, J=6.9 Hz, 6H). LCMS (ESI) calcd, for C₃₇H₃₆F₂N₁₁O₄⁺ [M+H]⁺: 736.29, found, 736.3.

### Example 226: Preparation of 1-(5-((4-(1-(6-((5-fluoro-4-(4-fluoro-1-isopropyl-2-methyl-1H-benzo[d]imidazol-6-yl)pyrimidin-2-yl)amino)pyridin-3-yl)piperidin-4-yl)piperazin-1-yl)methyl)-1-oxoisoindolin-2-yl)dihydropyrimidine-2,4 (1H,3H)-dione (GT-06736)

With reference to the method of Step 2 in Scheme T-1, the target compound (GT-06736) was obtained (yellow solid, 23 mg, yield 34.77%). ¹H NMR (400 MHz, DMSO-d₆) δ 12.34 (s, 1H), 11.75 (s, 1H), 10.69 (s, 1H), 8.86 (d, J=3.3 Hz, 1H), 8.29 (s, 1H), 8.21 (d, J=11.4 Hz, 1H), 7.94 (s, 2H), 7.85 (s, 2H), 7.78 (d, J=10.2 Hz, 2H), 4.92 (dd, J=13.8, 6.9 Hz, 1H), 4.80 (d, J=17.0 Hz, 1H), 4.54 (s, 3H), 3.83 (d, J=21.1 Hz, 7H), 3.57-3.50 (m, 6H), 2.81 (dd, J=21.0, 9.1 Hz, 4H), 2.74 (s, 3H), 2.22 (s, 2H), 1.86 (d, J=9.3 Hz, 2H), 1.64 (d, J=6.9 Hz, 6H). LCMS (ESI) calcd, for C₄₂H₄₇F₂N₁₂O₃⁺ [M+H]⁺: 805.39, found, 805.4.

### Example 227: Preparation of 2-(2,4-dioxotetrahydropyrimidin-1(2H)-yl)-5-((4-(1-(6-((5-fluoro-4-(4-fluoro-1-isopropyl-2-methyl-1H-benzo[d]imidazol-6-yl)pyrimidin-2-yl)amino)pyridin-3-yl)piperidin-4-yl)piperazin-1-yl)methyl)isoindoline-1,3-dione (GT-06737)

With reference to the method of Step 2 in Scheme T-1, the target compound (GT-06737) was obtained (yellow solid, 25 mg, yield 37.15%). ¹H NMR (400 MHz, DMSO-d₆) δ 12.21 (s, 1H), 11.73 (s, 1H), 10.93 (s, 1H), 8.87 (d, J=3.3 Hz, 1H), 8.31 (d, J=11.6 Hz, 2H), 8.19 (s, 2H), 8.09 (d, J=7.7 Hz, 1H), 7.95 (s, 1H), 7.83-7.75 (m, 2H), 4.92 (dt, J=13.7, 6.8 Hz, 1H), 4.48 (s, 2H), 3.93-3.83 (m, 6H), 3.46-3.29 (m, 7H), 2.90-2.78 (m, 4H), 2.74 (s, 3H), 2.24 (d, J=9.6 Hz, 2H), 1.87 (d, J=9.9 Hz, 2H), 1.65 (d, J=6.9 Hz, 6H). LCMS (ESI) calcd, for C₄₂H₄₅F₂N₁₂O₄⁺ [M+H]⁺: 819.37, found, 819.4.

### Example 228: Preparation of 2-((5-bromo-2-((4-((4-((2-(2,4-dioxotetrahydropyrimidin-1(2H)-yl)-1-oxoisoindolin-5-yl)methyl)piperazin-1-yl) sulfonyl)-2-methylphenyl)amino)pyrimidin-4-yl)amino)-6-fluorobenzamide (GT-06828)

With reference to the method of Step 2 in Scheme T-1, the target compound (GT-06828) was obtained (yellow solid, 28 mg, yield 48.09%). ¹H NMR (400 MHz, DMSO-d₆) δ 11.32 (s, 1H), 10.68 (s, 1H), 10.35 (s, 1H), 9.38 (s, 1H), 8.39 (s, 1H), 8.16 (s, 1H), 8.10 (s, 1H), 8.04 (d, J=8.3 Hz, 1H), 7.91 (d, J=8.5 Hz, 1H), 7.84 (d, J=7.7 Hz, 2H), 7.74 (d, J=8.0 Hz, 1H), 7.59 (s, 1H), 7.51 (dd, J=8.5, 1.8 Hz, 1H), 7.35 (d, J=6.6 Hz, 1H), 7.03 (s, 1H), 4.52 (d, J=33.9 Hz, 4H), 3.79 (s, 4H), 3.47-3.33 (m, 2H), 3.22 (s, 2H), 2.79 (dd, J=20.6, 15.2 Hz, 4H), 2.34 (s, 3H). LCMS (ESI) calcd, for C₃₅H₃₅BrFN₁₀O₆S⁺ [M+H]⁺: 821.16, found, 821.2.

### Example 229: Preparation of 2-((5-bromo-2-((4-((4-((2-(2,4-dioxotetrahydropyrimidin-1(2H)-yl)-1,3-dioxoisoindolin-5-yl)methyl)piperazin-1-yl) sulfonyl)-2-methylphenyl)amino)pyrimidin-4-yl)amino)-6-fluorobenzamide (GT-06829)

With reference to the method of Step 2 in Scheme T-1, the target compound (GT-06829) was obtained (yellow solid, 25 mg, yield 42.22%). ¹H NMR (400 MHz, DMSO-d₆) δ 11.40 (s, 1H), 10.92 (s, 1H), 10.31 (s, 1H), 9.28 (s, 1H), 8.37 (s, 1H), 8.24 (s, 1H), 8.16 (s, 1H), 8.09 (t, J=9.1 Hz, 4H), 7.91 (d, J=8.5 Hz, 1H), 7.60 (s, 1H), 7.52 (d, J=8.5 Hz, 1H), 7.34 (dd, J=14.9, 8.3 Hz, 1H), 7.02 (s, 1H), 4.57 (s, 2H), 3.82 (d, J=6.8 Hz, 2H), 3.66-3.57 (m, 3H), 3.37 (dd, J=39.6, 31.4 Hz, 4H), 2.85 (t, J=6.8 Hz, 2H), 2.75 (d, J=7.0 Hz, 1H), 2.34 (s, 3H). LCMS (ESI) calcd, for C₃₅H₃₃BrFN₁₀O₇S⁺ [M+H]⁺: 835.14, found, 835.2.

### Example 230: Preparation of 2-((5-bromo-2-((4-((4-((1-(2-(2,4-dioxotetrahydropyrimidin-1(2H)-yl)-1,3-dioxoisoindolin-5-yl)piperidin-4-yl)methyl)piperazin-1-yl) sulfonyl)-2-methylphenyl)amino)pyrimidin-4-yl)amino)-6-fluorobenzamide (GT-08337)

With reference to the method of Scheme T-3, the target compound (GT-08337) was obtained (yellow solid, 37 mg, yield 56.83%). ¹H NMR (400 MHz, DMSO-d₆) δ 10.84 (s, 1H), 10.32 (s, 1H), 10.19 (s, 1H), 9.30 (s, 1H), 8.38 (s, 1H), 8.17 (s, 1H), 8.11 (d, J=7.9 Hz, 2H), 7.94 (d, J=8.5 Hz, 1H), 7.71 (d, J=8.6 Hz, 1H), 7.64 (d, J=1.7 Hz, 1H), 7.56 (dd, J=8.5, 1.9 Hz, 1H), 7.45-7.32 (m, 2H), 7.29 (dd, J=8.8, 2.1 Hz, 1H), 7.12-7.00 (m, 1H), 4.08 (d, J=12.5 Hz, 2H), 3.79-3.71 (m, 4H), 3.59 (d, J=11.7 Hz, 2H), 3.16 (d, J=10.4 Hz, 2H), 3.08-2.93 (m, 4H), 2.84 (dd, J=17.9, 10.3 Hz, 4H), 2.37 (s, 3H), 2.13 (s, 1H), 1.84 (d, J=11.7 Hz, 2H), 1.22 (d, J=11.7 Hz, 2H). LCMS (ESI) calcd, for C₄₀H₄₂BrFN₁₁O₇S⁺ [M+H]⁺: 918.22, found, 918.3.

### Example 231: Preparation of 2-((5-bromo-2-((4-((4-((1-(2-(2,4-dioxotetrahydropyrimidin-1(2H)-yl)-1,3-dioxoisoindolin-4-yl)piperidin-4-yl)methyl)piperazin-1-yl) sulfonyl)-2-methylphenyl)amino)pyrimidin-4-yl)amino)-6-fluorobenzamide (GT-08338)

With reference to the method of Scheme T-3, the target compound (GT-08338) was obtained (yellow solid, 7 mg, yield 10.75%). ¹H NMR (400 MHz, DMSO-d₆) δ 10.84 (s, 1H), 10.28 (s, 1H), 10.00 (s, 1H), 9.21 (s, 1H), 8.35 (s, 1H), 8.13 (dd, J=19.6, 11.2 Hz, 3H), 7.95 (d, J=8.5 Hz, 1H), 7.78-7.69 (m, 1H), 7.64 (s, 1H), 7.60-7.53 (m, 1H), 7.37 (t, J=7.9 Hz, 3H), 7.11-6.99 (m, 1H), 3.80 (s, 2H), 3.72-3.57 (m, 6H), 3.19 (d, J=10.3 Hz, 2H), 3.09 (s, 2H), 2.96-2.78 (m, 6H), 2.37 (s, 3H), 2.02 (s, 1H), 1.88 (d, J=10.5 Hz, 2H), 1.52-1.28 (m, 2H). LCMS (ESI) calcd, for C₄₀H₄₂BrFN₁₁O₇S⁺ [M+H]⁺: 918.22, found, 918.3.

### Example 232: Preparation of 1-(5-((4-((4-((5-amino-1-(2,6-difluorobenzoyl)-1H-1,2,4-triazol-3-yl)amino)phenyl) sulfonyl)piperazin-1-yl)methyl)-1-oxoisoindolin-2-yl)dihydropyrimidine-2,4 (1H,3H)-dione (GT-06748)

With reference to the method of Step 2 in Scheme T-1, the target compound (GT-06748) was obtained (yellow solid, 30 mg, yield 48.23%). ¹H NMR (400 MHz, DMSO-d₆) δ 10.94 (s, 1H), 10.68 (s, 1H), 10.07 (s, 1H), 8.05 (s, 2H), 7.89-7.76 (m, 2H), 7.71 (dt, J=21.9, 7.5 Hz, 2H), 7.53 (dd, J=24.0, 8.9 Hz, 4H), 7.34 (t, J=8.3 Hz, 2H), 4.91-4.35 (m, 4H), 3.78 (t, J=6.9 Hz, 2H), 3.65 (s, 2H), 3.39 (s, 2H), 3.16 (s, 2H), 2.90-2.60 (m, 4H). LCMS (ESI) calcd, for C₃₂H₃₁F₂N₁₀O₆S⁺ [M+H]⁺: 721.21, found, 721.4.

### Example 233: Preparation of 5-((4-((4-((5-amino-1-(2,6-difluorobenzoyl)-1H-1,2,4-triazol-3-yl)amino)phenyl) sulfonyl)piperazin-1-yl)methyl)-2-(2,4-dioxotetrahydropyrimidin-1(2H)-yl)isoindoline-1,3-dione (GT-06749)

With reference to the method of Step 2 in Scheme T-1, the target compound (GT-06749) was obtained (yellow solid, 32 mg, yield 50.65%). ¹H NMR (400 MHz, DMSO-d₆) δ 10.92 (s, 2H), 10.06 (s, 1H), 8.16 (s, 1H), 8.03 (d, J=14.6 Hz, 4H), 7.77-7.63 (m, 1H), 7.53 (dd, J=21.9, 9.0 Hz, 4H), 7.34 (t, J=8.3 Hz, 2H), 4.49 (s, 2H), 3.81 (t, J=6.8 Hz, 2H), 3.64 (s, 1H), 3.50-3.40 (m, 4H), 3.16 (s, 2H), 2.85 (t, J=6.8 Hz, 2H), 2.72 (dd, J=21.6, 14.7 Hz, 2H). LCMS (ESI) calcd, for C₃₂H₂₉F₂N₁₀O₇S⁺ [M+H]⁺: 735.19, found, 735.4.

### Example 234: Preparation of 5-(4-((4-((4-((5-amino-1-(2,6-difluorobenzoyl)-1H-1,2,4-triazol-3-yl)amino)phenyl) sulfonyl)piperazin-1-yl)methyl)piperidin-1-yl)-2-(2,4-dioxotetrahydropyrimidin-1(2H)-yl)isoindoline-1,3-dione (GT-08339)

With reference to the method of Scheme T-3, the target compound (GT-08339) was obtained (yellow solid, 10 mg, yield 14.17%). ¹H NMR (400 MHz, DMSO-d₆) δ 10.83 (s, 1H), 10.09 (s, 1H), 9.91 (s, 1H), 8.06 (s, 2H), 7.78-7.68 (m, 2H), 7.57 (q, J=9.1 Hz, 4H), 7.36 (dd, J=14.2, 5.7 Hz, 3H), 7.28 (dd, J=8.8, 2.2 Hz, 1H), 4.08 (d, J=13.2 Hz, 2H), 3.77 (t, J=6.9 Hz, 2H), 3.62 (s, 2H), 3.55 (s, 2H), 3.11 (d, J=10.5 Hz, 2H), 2.98 (d, J=11.3 Hz, 4H), 2.82 (t, J=7.1 Hz, 2H), 2.73 (t, J=11.4 Hz, 2H), 2.09 (s, 1H), 1.82 (d, J=12.2 Hz, 2H), 1.20 (d, J=12.2 Hz, 2H). LCMS (ESI) calcd, for C₃₇H₃₈F₂N₁₁O₇S⁺ [M+H]⁺: 818.26, found, 818.3.

### Example 235: Preparation of 4-(4-((4-((4-((5-amino-1-(2,6-difluorobenzoyl)-1H-1,2,4-triazol-3-yl)amino)phenyl) sulfonyl)piperazin-1-yl)methyl)piperidin-1-yl)-2-(2,4-dioxotetrahydropyrimidin-1(2H)-yl)isoindoline-1,3-dione (GT-08340)

With reference to the method of Scheme T-3, the target compound (GT-08340) was obtained (yellow solid, 8 mg, yield 11.33%). ¹H NMR (400 MHz, DMSO-d₆) δ 10.84 (s, 1H), 10.09 (s, 1H), 9.58 (s, 1H), 8.05 (s, 2H), 7.78-7.68 (m, 2H), 7.58 (q, J=9.1 Hz, 4H), 7.36 (dd, J=15.5, 7.7 Hz, 4H), 3.73 (ddd, J=40.1, 17.2, 7.0 Hz, 6H), 3.57 (d, J=11.9 Hz, 2H), 3.21-3.03 (m, 4H), 2.93-2.78 (m, 4H), 2.70 (d, J=12.4 Hz, 2H), 1.98 (s, 1H), 1.84 (d, J=11.5 Hz, 2H), 1.40 (d, J=9.4 Hz, 2H). LCMS (ESI) calcd, for C₃₇H₃₈F₂N₁₁O₇S⁺ [M+H]⁺: 818.26, found, 818.3.

### Example 236: Preparation of 1-(5-((4-((4-((5-amino-1-(3-methylthiophene-2-carbonyl)-1H-1,2,4-triazol-3-yl)amino)phenyl) sulfonyl)piperazin-1-yl)methyl)-1-oxoisoindolin-2-yl)dihydropyrimidine-2,4 (1H,3H)-dione (GT-06852)

With reference to the method of Step 2 in Scheme T-1, the target compound (GT-06852) was obtained (yellow solid, 33 mg, yield 52.39%). ¹H NMR (500 MHz, DMSO) δ 10.70 (s, 1H), 10.11 (d, J=6.8 Hz, 1H), 8.02 (d, J=5.0 Hz, 1H), 7.86 (dd, J=39.6, 8.2 Hz, 5H), 7.72 (dt, J=21.6, 10.9 Hz, 3H), 7.59 (d, J=7.7 Hz, 1H), 7.17 (d, J=5.1 Hz, 1H), 4.64 (dd, J=114.3, 16.4 Hz, 2H), 4.35 (s, 2H), 3.76 (d, J=11.2 Hz, 4H), 3.13 (d, J=44.6 Hz, 6H), 2.86-2.72 (m, 2H), 2.62 (s, 3H). LCMS (ESI) calcd, for C₃₁H₃₃N₁₀O₆S₂⁺ [M+H]⁺: 705.20, found, 705.3.

### Example 237: Preparation of 5-((4-((4-((5-amino-1-(3-methylthiophene-2-carbonyl)-1H-1,2,4-triazol-3-yl)amino)phenyl) sulfonyl)piperazin-1-yl)methyl)-2-(2,4-dioxotetrahydropyrimidin-1(2H)-yl)isoindoline-1,3-dione (GT-06853)

With reference to the method of Step 2 in Scheme T-1, the target compound (GT-06853) was obtained (yellow solid, 30 mg, yield 46.70%). ¹H NMR (500 MHz, DMSO) δ 11.44 (s, 1H), 10.93 (s, 1H), 10.13 (s, 1H), 8.23 (s, 1H), 8.14-8.01 (m, 3H), 7.90 (d, J=8.8 Hz, 3H), 7.70 (d, J=9.0 Hz, 2H), 7.15 (d, J=5.0 Hz, 1H), 4.55 (s, 2H), 3.80 (t, J=6.8 Hz, 2H), 3.75-3.67 (m, 2H), 3.38 (s, 2H), 3.21 (s, 2H), 2.84 (t, J=6.8 Hz, 2H), 2.75 (dd, J=17.6, 11.9 Hz, 2H), 2.62 (s, 3H). LCMS (ESI) calcd, for C₃₁H₃₁N₁₀O₇S₂⁺ [M+H]⁺: 719.18, found, 719.2.

### Example 238: Preparation of 5-(4-((4-((4-((5-amino-1-(3-methylthiophene-2-carbonyl)-1H-1,2,4-triazol-3-yl)amino)phenyl) sulfonyl)piperazin-1-yl)methyl)piperidin-1-yl)-2-(2,4-dioxotetrahydropyrimidin-1(2H)-yl)isoindoline-1,3-dione (GT-08341)

With reference to the method of Scheme T-3, the target compound (GT-08341) was obtained (yellow solid, 36 mg, yield 66.96%). ¹H NMR (400 MHz, DMSO-d₆) δ 10.83 (s, 1H), 10.14 (s, 2H), 8.05 (d, J=5.0 Hz, 1H), 7.92 (t, J=9.9 Hz, 4H), 7.75 (d, J=9.0 Hz, 2H), 7.70 (d, J=8.6 Hz, 1H), 7.37 (d, J=2.1 Hz, 1H), 7.28 (dd, J=8.8, 2.2 Hz, 1H), 7.18 (d, J=5.1 Hz, 1H), 4.07 (d, J=12.8 Hz, 2H), 3.78 (d, J=6.9 Hz, 2H), 3.67-3.65 (m, 2H), 3.57 (d, J=11.3 Hz, 2H), 3.13 (d, J=10.9 Hz, 2H), 3.05-2.91 (m, 4H), 2.83 (dd, J=14.6, 8.3 Hz, 4H), 2.64 (s, 3H), 2.10 (s, 1H), 1.83 (d, J=11.8 Hz, 2H), 1.21 (d, J=12.5 Hz, 2H). LCMS (ESI) calcd. for C₃₆H₄₀N₁₁O₇S₂⁺ [M+H]⁺: 802.26, found, 802.3.

### Example 239: Preparation of 4-(4-((4-((4-((5-amino-1-(3-methylthiophene-2-carbonyl)-1H-1,2,4-triazol-3-yl)amino)phenyl) sulfonyl)piperazin-1-yl)methyl)piperidin-1-yl)-2-(2,4-dioxotetrahydropyrimidin-1(2H)-yl)isoindoline-1,3-dione (GT-08342)

With reference to the method of Step 2 in Scheme T-1, the target compound (GT-08342) was obtained (yellow solid, 28 mg, yield 52.08%). ¹H NMR (400 MHz, DMSO-d₆) δ 10.83 (s, 1H), 10.14 (s, 2H), 8.05 (d, J=5.0 Hz, 1H), 7.92 (t, J=10.5 Hz, 4H), 7.73 (dd, J=16.0, 8.4 Hz, 3H), 7.37 (d, J=7.6 Hz, 2H), 7.17 (d, J=5.1 Hz, 1H), 3.73 (dd, J=29.9, 7.5 Hz, 5H), 3.61 (s, 4H), 3.07 (s, 4H), 2.91-2.78 (m, 5H), 2.64 (s, 3H), 2.00 (s, 1H), 1.88 (d, J=12.0 Hz, 2H), 1.38 (dd, J=19.4, 9.5 Hz, 2H). LCMS (ESI) calcd, for C₃₆H₄₀N₁₁O₇S₂⁺ [M+H]⁺: 802.26, found, 802.3.

### Example 240: Preparation of 4-((7H-pyrrolo[2,3-d]pyrimidin-4-yl)amino)-N-(4-(4-(4-((2-(2,4-dioxotetrahydropyrimidin-1(2H)-yl)-1-oxoisoindolin-5-yl)methyl)piperazin-1-yl)piperidin-1-yl)phenyl)-1H-pyrazole-3-carboxamide (GT-08394)

With reference to the method of Step 2 in Scheme T-1, the target compound (GT-08394) was obtained (yellow solid, 27 mg, yield 44.15%). ¹H NMR (400 MHz, DMSO) δ 12.87 (s, 1H), 11.13 (s, 1H), 10.69 (s, 1H), 10.41 (s, 1H), 8.28 (d, J=5.8 Hz, 2H), 7.95 (s, 1H), 7.85 (s, 2H), 7.76 (d, J=8.4 Hz, 2H), 7.51 (s, 1H), 7.34 (s, 2H), 6.99 (s, 1H), 4.80-4.52 (m, 4H), 3.90-3.78 (m, 7H), 3.53-3.43 (m, 5H), 3.15 (s, 3H), 2.82 (dd, J=37.5, 6.7 Hz, 2H), 2.26 (s, 2H), 2.14 (s, 2H). LCMS (ESI) calcd, for C₃₈H₄₂N₁₃O₄⁺ [M+H]⁺: 744.35, found, 744.2.

### Example 241: Preparation of 4-((7H-pyrrolo[2,3-d]pyrimidin-4-yl)amino)-N-(4-(4-(4-((2-(2,4-dioxotetrahydropyrimidin-1(2H)-yl)-1,3-dioxoisoindolin-5-yl)methyl)piperazin-1-yl)piperidin-1-yl)phenyl)-1H-pyrazole-3-carboxamide (GT-08395)

With reference to the method of Step 2 in Scheme T-1, the target compound (GT-08395) was obtained (yellow solid, 37 mg, yield 59.39%). ¹H NMR (400 MHz, DMSO) δ 12.89 (s, 1H), 11.18 (s, 1H), 10.93 (s, 1H), 10.46 (d, J=16.7 Hz, 1H), 8.36 (s, 1H), 8.28 (d, J=7.6 Hz, 2H), 8.23 (d, J=7.7 Hz, 1H), 8.09 (d, J=7.7 Hz, 1H), 7.78 (d, J=8.7 Hz, 2H), 7.51 (s, 1H), 7.40 (s, 2H), 7.01 (s, 1H), 4.61 (s, 3H), 3.83 (t, J=6.7 Hz, 7H), 3.55-3.41 (m, 5H), 3.21 (s, 2H), 2.86 (t, J=6.7 Hz, 2H), 2.24 (d, J=39.7 Hz, 4H). LCMS (ESI) calcd. for C₃₈H₄₀N₁₃O₅⁺ [M+H]⁺: 758.33, found, 758.5.

### Example 242: Preparation of 4-((7H-pyrrolo[2,3-d]pyrimidin-4-yl)amino)-N-(4-(4-(4-((1-(2-(2,4-dioxotetrahydropyrimidin-1(2H)-yl)-1,3-dioxoisoindolin-5-yl)piperidin-4-yl)methyl)piperazin-1-yl)piperidin-1-yl)phenyl)-1H-pyrazole-3-carboxamide (GT-08396)

With reference to the method of Scheme T-3, the target compound (GT-08396) was obtained (yellow solid, 18 mg, yield 26.04%). ¹H NMR (400 MHz, DMSO) δ 12.89 (s, 1H), 11.70 (s, 1H), 11.16 (s, 1H), 10.84 (s, 1H), 10.44 (s, 1H), 8.28 (d, J=6.6 Hz, 2H), 7.78 (d, J=8.6 Hz, 2H), 7.72 (d, J=8.6 Hz, 1H), 7.51 (s, 1H), 7.41 (s, 3H), 7.32 (d, J=8.9 Hz, 1H), 4.13 (s, 2H), 3.86 (s, 4H), 3.57-3.45 (m, 6H), 3.26-2.97 (m, 8H), 2.83 (dd, J=8.0, 6.7 Hz, 2H), 2.41-2.12 (m, 6H), 2.03 (d, J=11.5 Hz, 2H), 1.27 (d, J=11.8 Hz, 2H). LCMS (ESI) calcd. for C₄₃H₄₉N₁₄O₅⁺ [M+H]⁺: 841.40, found, 841.2.

### Example 243: Preparation of 4-((7H-pyrrolo[2,3-d]pyrimidin-4-yl)amino)-N-(4-(4-(4-((1-(2-(2,4-dioxotetrahydropyrimidin-1(2H)-yl)-1,3-dioxoisoindolin-4-yl)piperidin-4-yl)methyl)piperazin-1-yl)piperidin-1-yl)phenyl)-1H-pyrazole-3-carboxamide (GT-08397)

With reference to the method of Scheme T-3, the target compound (GT-08397) was obtained (yellow solid, 33 mg, yield 47.74%). ¹H NMR (400 MHz, DMSO) δ 12.91 (s, 1H), 11.77 (s, 1H), 11.22 (s, 1H), 10.90 (d, J=44.1 Hz, 1H), 10.49 (s, 1H), 8.27 (d, J=8.8 Hz, 2H), 7.81 (d, J=8.7 Hz, 2H), 7.76-7.70 (m, 1H), 7.51 (s, 3H), 7.39 (t, J=8.7 Hz, 2H), 3.89 (s, 9H), 3.61 (d, J=35.2 Hz, 6H), 3.33 (s, 2H), 3.17 (s, 2H), 2.93 (dd, J=21.4, 11.0 Hz, 2H), 2.83 (t, J=6.8 Hz, 2H), 2.33 (s, 4H), 2.08 (s, 3H), 1.43 (dd, J=21.8, 11.5 Hz, 2H). LCMS (ESI) calcd, for C₄₃H₄₉N₁₄O₅⁺ [M+H]⁺: 841.40, found, 841.2.

### Example 244: Preparation of N-(4-((4-((1-(2-(2,4-dioxotetrahydropyrimidin-1(2H)-yl)-1,3-dioxoisoindolin-5-yl)piperidin-4-yl)methyl)piperazin-1-yl)methyl)-3-(trifluoromethyl)phenyl)-3-(imidazo[1,2-b]pyridazin-3-ylethynyl)-4-methylbenzamide (GT-08352)

With reference to the method of Scheme T-3, the target compound (GT-08352) was obtained (yellow solid, 20 mg, yield 31%). ¹H NMR (400 MHz, DMSO-d₆) δ 11.14 (s, 1H), 10.84 (s, 1H), 10.80 (s, 1H), 8.78 (d, J=4.4 Hz, 1H), 8.36 (s, 1H), 8.33-8.29 (m, 2H), 8.26-8.19 (m, 2H), 8.13 (s, 1H), 8.01 (d, J=8.0 Hz, 1H), 7.72 (d, J=8.6 Hz, 1H), 7.58 (d, J=8.2 Hz, 1H), 7.46 (dd, J=9.2, 4.5 Hz, 1H), 7.41 (s, 1H), 7.32 (d, J=8.7 Hz, 1H), 4.25 (s, 2H), 4.12 (d, J=13.1 Hz, 2H), 3.79-3.77 (m, 4H), 3.49-3.27 (m, 6H), 3.07 (s, 2H), 3.01 (t, J=12.1 Hz, 2H), 2.83 (t, J=7.0 Hz, 2H), 2.62 (s, 3H), 2.17 (s, 1H), 1.98 (d, J=12.1 Hz, 2H), 1.34-1.19 (m, 2H). LCMS (ESI) calcd, for C₄₆H₄₄F₃N₁₀O₅⁺ [M+H]⁺: 873.34, found, 873.2.

### Example 245: Preparation of N-(4-((4-((1-(2-(2,4-dioxotetrahydropyrimidin-1(2H)-yl)-1,3-dioxoisoindolin-4-yl)piperidin-4-yl)methyl)piperazin-1-yl)methyl)-3-(trifluoromethyl)phenyl)-3-(imidazo[1,2-b]pyridazin-3-ylethynyl)-4-methylbenzamide (GT-08353)

With reference to the method of Scheme T-3, the target compound (GT-08353) was obtained (yellow solid, 20 mg, yield 31%). ¹H NMR (400 MHz, DMSO-d₆) δ 10.85 (s, 1H), 10.76 (s, 1H), 8.77 (dd, J=4.4, 1.4 Hz, 1H), 8.35-8.27 (m, 3H), 8.25-8.17 (m, 2H), 8.05 (s, 1H), 8.01-7.92 (m, 1H), 7.75 (t, 1H), 7.58 (d, J=8.3 Hz, 1H), 7.45 (dd, J=9.2, 4.5 Hz, 1H), 7.40 (t, J=7.6 Hz, 2H), 4.15 (s, 2H), 3.85-3.80 (m, 2H), 3.79-3.76 (m, 4H), 3.47-3.21 (m, 6H), 3.13 (s, 2H), 2.98-2.88 (m, 2H), 2.87-2.81 (m, 2H), 2.62 (s, 3H), 2.10-1.96 (m, 3H), 1.55-1.38 (m, 2H). LCMS (ESI) calcd, for C₄₆H₄₄F₃N₁₀O₅⁺ [M+H]⁺: 873.34, found, 873.2.

### Example 246: Preparation of N-(4-(4-((1-(2-(2,4-dioxotetrahydropyrimidin-1(2H)-yl)-1,3-dioxoisoindolin-5-yl)piperidin-4-yl)methyl)piperazin-1-yl)-3-(trifluoromethyl)phenyl)-3-(imidazo[1,2-b]pyridazin-3-ylethynyl)-4-methylbenzamide (GT-08354)

With reference to the method of Scheme T-3, the target compound (GT-08354) was obtained (yellow solid, 30 mg, yield 46%). ¹H NMR (400 MHz, DMSO-d₆) δ 10.85 (s, 1H), 10.64 (s, 1H), 10.49 (s, 1H), 8.83-8.70 (m, 1H), 8.35-8.27 (m, 2H), 8.26-8.20 (m, 2H), 8.16 (d, J=8.7 Hz, 1H), 8.02-7.95 (m, 1H), 7.73 (d, J=8.6 Hz, 1H), 7.58 (dd, J=12.5, 8.7 Hz, 2H), 7.48-7.40 (m, 2H), 7.33 (d, J=8.7 Hz, 1H), 4.14 (d, J=13.4 Hz, 2H), 3.78 (t, J=6.8 Hz, 2H), 3.59 (s, 3H), 3.46-3.37 (m, 2H), 3.16-3.10 (m, 3H), 3.09-3.00 (m, 4H), 2.84 (t, J=7.2 Hz, 2H), 2.62 (s, 3H), 2.22 (s, 1H), 1.97 (d, J=11.5 Hz, 2H), 1.31 (d, J=11.3 Hz, 2H). LCMS (ESI) calcd, for C₄₅H₄₂F₃N₁₀O₅⁺ [M+H]⁺: 859.33, found, 859.1.

### Example 247: Preparation of N-(4-(4-((1-(2-(2,4-dioxotetrahydropyrimidin-1(2H)-yl)-1,3-dioxoisoindolin-4-yl)piperidin-4-yl)methyl)piperazin-1-yl)-3-(trifluoromethyl)phenyl)-3-(imidazo[1,2-b]pyridazin-3-ylethynyl)-4-methylbenzamide (GT-08355)

With reference to the method of Scheme T-3, the target compound (GT-08355) was obtained (yellow solid, 25 mg, yield 38%). ¹H NMR (400 MHz, DMSO-d₆) δ 10.86 (s, 1H), 10.64 (s, 1H), 10.51 (s, 1H), 8.76 (dd, J=4.4, 1.5 Hz, 1H), 8.37-8.27 (m, 2H), 8.26-8.19 (m, 2H), 8.18-8.09 (m, 1H), 7.98 (dd, J=8.0, 1.7 Hz, 1H), 7.81-7.68 (m, 1H), 7.58 (dd, J=13.9, 8.6 Hz, 2H), 7.49-7.34 (m, 3H), 3.85-3.76 (m, 3H), 3.62 (s, 4H), 3.47-3.37 (m, 2H), 3.23-3.14 (m, 3H), 3.06 (d, J=11.6 Hz, 2H), 3.01-2.89 (m, 2H), 2.88-2.79 (m, 2H), 2.62 (s, 3H), 2.11 (s, 1H), 2.01 (d, J=12.1 Hz, 2H), 1.59-1.38 (m, 2H). LCMS (ESI) calcd. for C₄₅H₄₂F₃N₁₀O₅⁺ [M+H]⁺: 859.33, found, 859.2.

### Example 248: Preparation of N-(4-(chlorodifluoromethoxy)phenyl)-6-(4-((1-(2-(2,4-dioxotetrahydropyrimidin-1(2H)-yl)-1,3-dioxoisoindolin-5-yl)piperidin-4-yl)methyl)piperazin-1-yl)-5-(1H-pyrazol-3-yl)nicotinamide (GT-08356)

With reference to the method of Scheme T-3, the target compound (GT-08356) was obtained (yellow solid, 26 mg, yield 38%). ¹H NMR (400 MHz, DMSO-d₆) δ 10.84 (s, 1H), 10.55 (s, 1H), 10.46 (s, 1H), 8.83 (d, J=2.3 Hz, 1H), 8.46 (d, J=2.2 Hz, 1H), 7.92 (d, J=9.0 Hz, 2H), 7.86 (d, J=1.9 Hz, 1H), 7.72 (d, J=8.6 Hz, 1H), 7.41 (s, 1H), 7.36 (d, J=8.7 Hz, 2H), 7.31 (d, J=8.8 Hz, 1H), 6.81 (d, J=2.0 Hz, 1H), 4.12 (d, J=12.5 Hz, 2H), 3.78 (t, J=6.8 Hz, 3H), 3.66 (d, 3H), 3.52 (d, 2H), 3.41-3.35 (m, 2H), 3.16-3.12 (m, 1H), 3.06-2.97 (m, 3H), 2.83 (t, J=7.1 Hz, 2H), 2.18 (s, 1H), 1.92 (d, J=11.1 Hz, 2H), 1.33-1.21 (m, 2H). LCMS (ESI) calcd, for C₄₃H₅₃FN₇O₉⁺ [M+H]⁺: 803.26, found, 803.1.

### Example 249: Preparation of N-(4-(chlorodifluoromethoxy)phenyl)-6-(4-((1-(2-(2,4-dioxotetrahydropyrimidin-1(2H)-yl)-1,3-dioxoisoindolin-4-yl)piperidin-4-yl)methyl)piperazin-1-yl)-5-(1H-pyrazol-3-yl)nicotinamide (GT-08357)

With reference to the method of Scheme T-3, the target compound (GT-08357) was obtained (yellow solid, 25 mg, yield 34%). ¹H NMR (400 MHz, DMSO-d₆) δ 10.85 (s, 1H), 10.56 (s, 2H), 8.84 (d, J=2.1 Hz, 1H), 8.46 (d, J=2.1 Hz, 1H), 7.92 (d, J=9.0 Hz, 2H), 7.86 (d, J=1.4 Hz, 1H), 7.74 (t, J=7.8 Hz, 1H), 7.44-7.30 (m, 4H), 6.82 (d, J=1.6 Hz, 1H), 3.81-3.76 (m, 3H), 3.68 (d, J=12.9 Hz, 3H), 3.55 (d, J=11.0 Hz, 2H), 3.45-3.38 (m, 2H), 3.21-3.08 (m, 4H), 2.98-2.87 (m, 2H), 2.86-2.78 (m, 2H), 2.08 (s, 1H), 1.99 (d, J=11.9 Hz, 2H), 1.53-1.37 (m, 2H). LCMS (ESI) calcd, for C₃₈H₃₈ClF₂N₁₀O₆⁺ [M+H]⁺: 803.26, found, 803.1.

### Example 250: Preparation of N-(4-(chlorodifluoromethoxy)phenyl)-6-(4-(1-((1-(2-(2,4-dioxotetrahydropyrimidin-1(2H)-yl)-1,3-dioxoisoindolin-5-yl)piperidin-4-yl)methyl)piperidin-4-yl)piperazin-1-yl)-5-(1H-pyrazol-3-yl)nicotinamide (GT-08358)

With reference to the method of Scheme T-3, the target compound (GT-08358) was obtained (yellow solid, 17 mg, yield 27%). ¹H NMR (400 MHz, DMSO-d₆) δ 11.57 (s, 1H), 10.84 (s, 1H), 10.57 (s, 1H), 10.24 (s, 1H), 8.84 (d, J=2.3 Hz, 1H), 8.46 (d, J=2.3 Hz, 1H), 7.92 (d, J=9.1 Hz, 2H), 7.84 (d, J=1.9 Hz, 1H), 7.73 (d, J=8.6 Hz, 1H), 7.44-7.29 (m, 4H), 6.88-6.78 (m, 1H), 4.12 (d, J=13.0 Hz, 2H), 3.78 (t, J=6.9 Hz, 3H), 3.67 (d, 4H), 3.40-3.35 (m, 2H), 3.21-3.09 (m, 3H), 3.04-2.91 (m, 5H), 2.83 (t, J=7.1 Hz, 2H), 2.38-2.25 (m, 4H), 2.18 (s, 1H), 1.92 (d, J=10.9 Hz, 2H), 1.34-1.18 (m, 2H). LCMS (ESI) calcd, for C₄₃H₄₇ClF₂N₁₁O₆⁺ [M+H]⁺: 886.34, found, 886.2.

### Example 251: Preparation of N-(4-(chlorodifluoromethoxy)phenyl)-6-(4-(1-((1-(2-(2,4-dioxotetrahydropyrimidin-1(2H)-yl)-1,3-dioxoisoindolin-4-yl)piperidin-4-yl)methyl)piperidin-4-yl)piperazin-1-yl)-5-(1H-pyrazol-3-yl)nicotinamide (GT-08359)

With reference to the method of Scheme T-3, the target compound (GT-08359) was obtained (yellow solid, 21 mg, yield 34%). ¹H NMR (400 MHz, DMSO-d₆) δ 11.61 (s, 1H), 10.86 (s, 1H), 10.58 (s, 1H), 10.33 (s, 1H), 8.84 (d, J=2.2 Hz, 1H), 8.47 (d, J=2.2 Hz, 1H), 7.93 (d, J=9.0 Hz, 2H), 7.85 (d, J=2.0 Hz, 1H), 7.75 (t, J=7.8 Hz, 1H), 7.42-7.34 (m, 4H), 6.84 (d, J=2.1 Hz, 1H), 3.81-3.77 (m, 5H), 3.74-3.71 (m, 5H), 3.50 (d, 2H), 3.43-3.37 (m, 2H), 3.20-3.14 (m, 2H), 3.05-2.93 (m, 5H), 2.86-2.80 (m, 2H), 2.34 (s, 4H), 2.08 (s, 1H), 1.98 (d, J=11.0 Hz, 2H), 1.50-1.37 (m, 2H). LCMS (ESI) calcd, for C₄₃H₄₇ClF₂N₁₁O₆⁺ [M+H]⁺: 886.34, found, 886.2.

### Example 252: Preparation of N-(4-(chlorodifluoromethoxy)phenyl)-6-(4-(4-((1-(2-(2,4-dioxotetrahydropyrimidin-1(2H)-yl)-1,3-dioxoisoindolin-5-yl)piperidin-4-yl)methyl)piperazin-1-yl)piperidin-1-yl)-5-(1H-pyrazol-3-yl)nicotinamide (GT-08360)

With reference to the method of Scheme T-3, the target compound (GT-08360) was obtained (yellow solid, 25 mg, yield 40%). ¹H NMR (400 MHz, DMSO-d₆) δ 10.84 (s, 1H), 10.50 (s, 1H), 8.78 (d, J=5.4 Hz, 1H), 8.38 (d, J=2.3 Hz, 1H), 7.91 (d, J=9.1 Hz, 2H), 7.82 (d, J=1.9 Hz, 1H), 7.72 (d, J=8.6 Hz, 1H), 7.43-7.29 (m, 4H), 6.71 (d, J=1.9 Hz, 1H), 4.12 (d, J=13.0 Hz, 2H), 3.80-3.76 (m, 6H), 3.72 (s, 4H), 3.49-3.45 (m, 3H), 3.11-2.96 (m, 4H), 2.86-2.75 (m, 4H), 2.19 (s, 1H), 2.09 (d, 2H), 2.00 (d, J=11.3 Hz, 2H), 1.87-1.76 (m, 2H), 1.26 (d, J=10.2 Hz, 2H). LCMS (ESI) calcd, for C₄₃H₄₇ClF₂N₁₁O₆⁺ [M+H]⁺: 886.34, found, 886.2.

### Example 253: Preparation of N-(4-(chlorodifluoromethoxy)phenyl)-6-(4-(4-((1-(2-(2,4-dioxotetrahydropyrimidin-1(2H)-yl)-1,3-dioxoisoindolin-4-yl)piperidin-4-yl)methyl)piperazin-1-yl)piperidin-1-yl)-5-(1H-pyrazol-3-yl)nicotinamide (GT-08361)

With reference to the method of Scheme T-3, the target compound (GT-08361) was obtained (yellow solid, 25 mg, yield 40%). ¹H NMR (400 MHz, DMSO-d₆) δ 12.12 (s, 1H), 11.57 (s, 1H), 10.85 (s, 1H), 10.51 (s, 1H), 8.79 (d, J=5.6 Hz, 1H), 8.39 (d, J=2.3 Hz, 1H), 7.91 (d, J=9.1 Hz, 2H), 7.83 (d, J=2.0 Hz, 1H), 7.77-7.71 (m, 1H), 7.44-7.33 (m, 4H), 6.78-6.65 (m, 1H), 3.90-3.87 (m, 2H), 3.81-3.77 (m, 6H), 3.71-3.66 (m, 3H), 3.55-3.44 (m, 4H), 3.15 (s, 2H), 2.98-2.89 (m, 2H), 2.86-2.76 (m, 4H), 2.14-1.99 (m, 5H), 1.87-1.74 (m, 2H), 1.53-1.38 (m, 2H). LCMS (ESI) calcd, for C₄₃H₄₇ClF₂N₁₁O₆⁺ [M+H]⁺: 886.34, found, 886.2.

### Example 254: Preparation of N-(4-((4-((2-(2,4-dioxotetrahydropyrimidin-1(2H)-yl)-1-oxoisoindolin-5-yl)methyl)piperazin-1-yl)methyl)-3-(trifluoromethyl)phenyl)-3-(imidazo[1,2-b]pyridazin-3-ylethynyl)-4-methylbenzamide (GT-08216)

With reference to the method of Scheme T-3, the target compound (GT-08216) was obtained (white solid, 32 mg, yield 55%). ¹H NMR (400 MHz, DMSO-d₆) δ 10.74 (s, 1H), 10.69 (s, 1H), 8.77 (d, J=4.4, 1.4 Hz, 1H), 8.37-8.28 (m, 3H), 8.23 (d, J=1.6 Hz, 1H), 8.18 (d, J=8.6 Hz, 1H), 8.07-7.93 (m, 2H), 7.91 (s, 1H), 7.86 (d, J=7.8 Hz, 1H), 7.80 (d, J=7.9 Hz, 1H), 7.57 (d, J=8.3 Hz, 1H), 7.46 (dd, J=9.2, 4.5 Hz, 1H), 4.81 (d, J=16.7 Hz, 1H), 4.62-4.45 (m, 3H), 4.10 (s, 2H), 3.79 (t, 3H), 3.42 (s, 3H), 3.34-3.12 (m, 4H), 2.92-2.83 (m, 1H), 2.81-2.72 (m, 1H), 2.62 (s, 3H). LCMS (ESI) calcd, for C₄₁H₃₇F₃N₉O₄⁺ [M+H]⁺: 776.29, found, 776.1.

### Example 255: Preparation of N-(4-((4-((2-(2,4-dioxotetrahydropyrimidin-1(2H)-yl)-1,3-dioxoisoindolin-5-yl)methyl)piperazin-1-yl)methyl)-3-(trifluoromethyl)phenyl)-3-(imidazo[1,2-b]pyridazin-3-ylethynyl)-4-methylbenzamide (GT-08217)

With reference to the method of Scheme T-3, the target compound (GT-08217) was obtained (white solid, 35 mg, yield 59%). ¹H NMR (400 MHz, DMSO-d₆) δ 10.93 (s, 1H), 10.74 (s, 1H), 8.77 (dd, J=4.4, 1.4 Hz, 1H), 8.34-8.27 (m, 4H), 8.23 (s, 1H), 8.18 (d, J=8.2 Hz, 2H), 8.09 (d, J=7.7 Hz, 1H), 8.07-7.92 (m, 2H), 7.57 (d, J=8.3 Hz, 1H), 7.46 (dd, J=9.2, 4.5 Hz, 1H), 4.53 (s, 2H), 4.08 (s, 2H), 3.82 (t, J=6.7 Hz, 3H), 3.44-3.31 (m, 3H), 3.30-3.07 (m, 4H), 2.86 (t, J=6.8 Hz, 2H), 2.62 (s, 3H). LCMS (ESI) calcd. for C₄₁H₃₅F₃N₉O₅⁺ [M+H]⁺: 790.27, found, 790.1.

### Example 256: Preparation of N-(4-(4-((2-(2,4-dioxotetrahydropyrimidin-1(2H)-yl)-1-oxoisoindolin-5-yl)methyl)piperazin-1-yl)-3-(trifluoromethyl)phenyl)-3-(imidazo[1,2-b]pyridazin-3-ylethynyl)-4-methylbenzamide (GT-08218)

With reference to the method of Scheme T-3, the target compound (GT-08218) was obtained (white solid, 34 mg, yield 58%). ¹H NMR (400 MHz, DMSO-d₆) δ 11.29 (s, 1H), 10.70 (s, 1H), 10.62 (s, 1H), 8.76 (dd, J=4.4, 1.3 Hz, 1H), 8.28 (s, 1H), 8.23 (dd, J=6.6, 1.9 Hz, 2H), 8.16-8.11 (m, 1H), 8.01-7.94 (m, 2H), 7.91-7.84 (m, 2H), 7.56 (d, J=8.1 Hz, 2H), 7.43 (dd, J=9.2, 4.5 Hz, 1H), 4.83 (d, J=17.0 Hz, 1H), 4.60 (d, J=15.6 Hz, 3H), 3.80 (t, J=6.8 Hz, 2H), 3.41 (s, 2H), 3.35-3.25 (m, 2H), 3.25-3.15 (m, 2H), 3.06 (d, J=11.3 Hz, 2H), 2.93-2.84 (m, 1H), 2.82-2.74 (m, 1H), 2.61 (s, 3H). LCMS (ESI) calcd, for C₄₀H₃₅F₃N₉O₄⁺ [M+H]⁺: 762.28, found, 762.2.

### Example 257: Preparation of N-(4-(4-((2-(2,4-dioxotetrahydropyrimidin-1(2H)-yl)-1,3-dioxoisoindolin-5-yl)methyl)piperazin-1-yl)-3-(trifluoromethyl)phenyl)-3-(imidazo[1,2-b]pyridazin-3-ylethynyl)-4-methylbenzamide (GT-08219)

With reference to the method of Scheme T-3, the target compound (GT-08219) was obtained (white solid, 32 mg, yield 54%). ¹H NMR (400 MHz, DMSO-d₆) δ 11.52 (s, 1H), 10.94 (s, 1H), 10.56 (d, J=53.1 Hz, 1H), 8.73 (d, J=18.7 Hz, 1H), 8.39 (s, 1H), 8.35-8.20 (m, 5H), 8.17-8.09 (m, 2H), 7.97 (d, J=7.9 Hz, 1H), 7.61-7.52 (m, 2H), 7.43 (dd, J=9.2, 4.4 Hz, 1H), 4.67 (s, 2H), 3.83 (t, J=6.7 Hz, 2H), 3.42 (d, J=9.8 Hz, 2H), 3.37-3.27 (m, 2H), 3.22 (s, 2H), 3.13-3.02 (m, 2H), 2.87 (t, J=6.7 Hz, 2H), 2.61 (s, 3H). LCMS (ESI) calcd, for C₄₀H₃₃F₃N₉O₅⁺ [M+H]⁺: 776.26, found, 776.2.

### Example 258: Preparation of N-(4-(4-(((2-(2,4-dioxotetrahydropyrimidin-1(2H)-yl)-1-oxoisoindolin-5-yl)methyl)(methyl)amino)piperidin-1-yl)-3- (trifluoromethyl)phenyl)-3-(imidazo[1,2-b]pyridazin-3-ylethynyl)-4-methylbenzamide (GT-08220)

With reference to the method of Scheme T-3, the target compound (GT-08220) was obtained (white solid, 13 mg, yield 23%). ¹H NMR (400 MHz, DMSO-d₆) δ 10.95 (s, 1H), 10.71 (s, 1H), 10.59 (s, 1H), 8.79-8.74 (m, 1H), 8.33-8.26 (m, 2H), 8.20 (d, 2H), 8.10 (d, J=8.7 Hz, 1H), 7.98 (d, J=6.1 Hz, 2H), 7.91-7.80 (m, 2H), 7.62-7.53 (m, 2H), 7.45 (dd, J=9.2, 4.4 Hz, 1H), 4.89-4.76 (m, 1H), 4.72-4.55 (m, 2H), 4.40 (dd, J=12.6, 6.9 Hz, 1H), 3.83-3.81 (m, 2H), 3.36 (t, 1H), 3.05 (s, 2H), 2.94-2.77 (m, 4H), 2.66 (d, J=4.5 Hz, 3H), 2.62 (s, 3H), 2.34-2.21 (m, 2H), 1.99-1.81 (m, 2H). LCMS (ESI) calcd, for C₄₂H₃₉F₃N₉O₄⁺ [M+H]⁺: 790.31, found, 790.1.

### Example 259: Preparation of N-(4-(4-(((2-(2,4-dioxotetrahydropyrimidin-1(2H)-yl)-1,3-dioxoisoindolin-5-yl)methyl)(methyl)amino)piperidin-1-yl)-3-(trifluoromethyl)phenyl)-3-(imidazo[1,2-b]pyridazin-3-ylethynyl)-4-methylbenzamide (GT-08221)

With reference to the method of Scheme T-3, the target compound (GT-08221) was obtained (white solid, 28 mg, yield 48%). ¹H NMR (400 MHz, DMSO-d₆) δ 11.10 (d, J=25.9 Hz, 1H), 10.94 (s, 1H), 10.59 (s, 1H), 8.77 (dd, J=4.4, 1.5 Hz, 1H), 8.40 (d, J=4.3 Hz, 1H), 8.32-8.25 (m, 3H), 8.22 (d, J=1.6 Hz, 1H), 8.19 (d, J=2.4 Hz, 1H), 8.13-8.08 (m, 2H), 7.98 (d, J=8.0, 1.6 Hz, 1H), 7.61-7.53 (m, 2H), 7.44 (dd, J=9.2, 4.5 Hz, 1H), 4.81-4.67 (m, 1H), 4.58-4.41 (m, 1H), 3.84 (t, J=6.7 Hz, 2H), 3.43 (s, 1H), 3.07 (s, 2H), 2.87 (t, J=6.8 Hz, 4H), 2.66 (d, J=3.2 Hz, 3H), 2.62 (s, 3H), 2.29 (s, 2H), 2.01-1.80 (m, 2H). LCMS (ESI) calcd. for C₄₂H₃₇F₃N₉O₅⁺ [M+H]⁺: 804.29, found, 804.2.

### Example 260: Preparation of N-(4-(chlorodifluoromethoxy)phenyl)-6-(4-((2-(2,4-dioxotetrahydropyrimidin-1(2H)-yl)-1-oxoisoindolin-5-yl)methyl)piperazin-1-yl)-5-(1H-pyrazol-3-yl)nicotinamide (GT-08222)

With reference to the method of Scheme T-3, the target compound (GT-08222) was obtained (white solid, 36 mg, yield 60%). ¹H NMR (400 MHz, DMSO-d₆) δ 11.52 (s, 1H), 10.69 (s, 1H), 10.57 (s, 1H), 8.83 (d, J=2.2 Hz, 1H), 8.46 (d, J=5.4 Hz, 1H), 7.96-7.89 (m, 3H), 7.89-7.84 (m, 2H), 7.82 (d, J=8.0 Hz, 1H), 7.36 (d, J=8.8 Hz, 2H), 6.81 (d, J=2.0 Hz, 1H), 4.81 (d, J=16.7 Hz, 1H), 4.56 (s, 1H), 4.51 (s, 2H), 3.79 (t, J=6.8 Hz, 2H), 3.69 (d, J=12.6 Hz, 2H), 3.39-3.27 (m, 4H), 3.25-3.14 (m, 2H), 2.92-2.82 (m, 1H), 2.80-2.70 (m, 1H). LCMS (ESI) calcd, for C₃₃H₃₁ClF₂N₉O₅⁺ [M+H]⁺: 706.21, found, 706.1.

### Example 261: Preparation of N-(4-(chlorodifluoromethoxy)phenyl)-6-(4-((2-(2,4-dioxotetrahydropyrimidin-1(2H)-yl)-1,3-dioxoisoindolin-5-yl)methyl)piperazin-1-yl)-5-(1H-pyrazol-3-yl)nicotinamide (GT-08223)

With reference to the method of Scheme T-3, the target compound (GT-08223) was obtained (white solid, 23 mg, yield 37%). ¹H NMR (400 MHz, DMSO-d₆) δ 11.62 (s, 1H), 10.93 (s, 1H), 10.56 (s, 1H), 8.83 (d, J=2.3 Hz, 1H), 8.46 (d, J=2.3 Hz, 1H), 8.33 (s, 1H), 8.19 (d, J=8.8 Hz, 1H), 8.10 (d, J=7.7 Hz, 1H), 7.91 (d, J=9.1 Hz, 2H), 7.86 (d, J=2.1 Hz, 1H), 7.36 (d, J=9.0 Hz, 2H), 6.80 (d, J=2.2 Hz, 1H), 4.60 (s, 2H), 3.82 (t, J=6.7 Hz, 2H), 3.73-3.67 (m, 2H), 3.40-3.28 (m, 4H), 3.23 (s, 2H), 2.86 (t, J=6.8 Hz, 2H). LCMS (ESI) calcd, for C₃₃H₂₉ClF₂N₉O₆⁺ [M+H]⁺: 720.19, found, 720.1.

### Example 262: Preparation of N-(4-(chlorodifluoromethoxy)phenyl)-6-(4-(1-((2-(2,4-dioxotetrahydropyrimidin-1(2H)-yl)-1-oxoisoindolin-5-yl)methyl)piperidin-4-yl)piperazin-1-yl)-5-(1H-pyrazol-3-yl)nicotinamide (GT-08224)

With reference to the method of Scheme T-3, the target compound (GT-08224) was obtained (white solid, 25 mg, yield 44%). ¹H NMR (400 MHz, DMSO-d₆) δ 11.81-11.43 (m, 1H), 11.29 (s, 1H), 10.70 (s, 1H), 10.59 (s, 1H), 8.83 (d, J=2.4 Hz, 1H), 8.46 (d, J=2.4 Hz, 1H), 7.97-7.82 (m, 5H), 7.79 (d, J=8.0 Hz, 1H), 7.36 (d, J=8.9 Hz, 2H), 6.83 (t, J=2.8 Hz, 1H), 4.82 (d, J=16.5 Hz, 1H), 4.58 (d, J=16.3 Hz, 2H), 4.46 (s, 2H), 3.79 (t, J=6.8 Hz, 2H), 3.71 (d, J=12.4 Hz, 2H), 3.55-3.41 (m, 5H), 3.35 (t, J=12.3 Hz, 2H), 3.21-3.11 (m, 2H), 2.96 (s, 2H), 2.90-2.82 (m, 1H), 2.81-2.71 (m, 1H), 2.37-2.33 (m, 1H), 2.28-2.10 (m, 2H). LCMS (ESI) calcd, for C₃₈H₄₀ClF₂N₁₀O₅⁺ [M+H]⁺: 789.28, found, 789.1.

### Example 263: Preparation of N-(4-(chlorodifluoromethoxy)phenyl)-6-(4-(1-((2-(2,4-dioxotetrahydropyrimidin-1(2H)-yl)-1,3-dioxoisoindolin-5-yl)methyl)piperidin-4-yl)piperazin-1-yl)-5-(1H-pyrazol-3-yl)nicotinamide (GT-08225)

With reference to the method of Scheme T-3, the target compound (GT-08225) was obtained (white solid, 26 mg, yield 45%). ¹H NMR (400 MHz, DMSO-d₆) δ 11.45 (d, J=42.7 Hz, 1H), 10.93 (s, 1H), 10.58 (s, 1H), 8.83 (d, J=2.2 Hz, 1H), 8.46 (d, J=2.2 Hz, 1H), 8.33 (d, J=15.9 Hz, 1H), 8.18 (d, J=7.9 Hz, 1H), 8.11 (d, J=7.6 Hz, 1H), 7.92 (d, J=9.1 Hz, 2H), 7.84 (d, J=1.9 Hz, 1H), 7.36 (d, J=8.7 Hz, 2H), 6.82 (d, J=2.0 Hz, 1H), 4.55 (s, 2H), 3.83 (t, J=6.7 Hz, 2H), 3.71 (d, J=12.5 Hz, 2H), 3.58-3.44 (m, 5H), 3.35 (t, J=11.9 Hz, 2H), 3.20-3.10 (m, 2H), 3.02 (s, 2H), 2.86 (t, J=6.8 Hz, 2H), 2.34 (d, 2H), 2.26-2.09 (m, 2H). LCMS (ESI) calcd, for C₃₈H₃₈ClF₂N₁₀O₆⁺ [M+H]⁺: 803.26, found, 803.1.

### Example 264: Preparation of N-(4-(chlorodifluoromethoxy)phenyl)-6-(4-(4-((2-(2,4-dioxotetrahydropyrimidin-1(2H)-yl)-1-oxoisoindolin-5-yl)methyl)piperazin-1-yl)piperidin-1-yl)-5-(1H-pyrazol-3-yl)nicotinamide (GT-08226)

With reference to the method of Scheme T-3, the target compound (GT-08226) was obtained (white solid, 21 mg, yield 37%). ¹H NMR (400 MHz, DMSO-d₆) δ 10.69 (s, 1H), 10.49 (s, 1H), 8.78 (d, J=2.3 Hz, 1H), 8.37 (d, J=2.3 Hz, 1H), 7.93-7.84 (m, 4H), 7.81 (d, J=1.9 Hz, 2H), 7.35 (d, J=8.8 Hz, 2H), 6.69 (d, J=2.1 Hz, 1H), 4.80 (d, J=16.2 Hz, 1H), 4.56 (d, J=16.9 Hz, 1H), 4.50 (s, 1H), 3.79 (t, 6H), 3.70-3.66 (m, 3H), 3.57-3.52 (m, 3H), 3.44-3.39 (m, 2H), 2.90-2.83 (m, 1H), 2.78 (t, J=12.0 Hz, 3H), 2.07 (d, J=11.7 Hz, 2H), 1.82-1.70 (m, 2H). LCMS (ESI) calcd, for C₃₈H₄₀ClF₂N₁₀O₅⁺ [M+H]⁺: 789.28, found, 789.2.

### Example 265: Preparation of N-(4-(chlorodifluoromethoxy)phenyl)-6-(4-(4-((2-(2,4-dioxotetrahydropyrimidin-1(2H)-yl)-1,3-dioxoisoindolin-5-yl)methyl)piperazin-1-yl)piperidin-1-yl)-5-(1H-pyrazol-3-yl)nicotinamide (GT-08227)

With reference to the method of Scheme T-3, the target compound (GT-08227) was obtained (white solid, 25 mg, yield 44%). ¹H NMR (400 MHz, DMSO-d₆) δ 11.75 (s, 1H), 10.93 (s, 1H), 10.50 (s, 1H), 8.78 (d, J=2.3 Hz, 1H), 8.38 (d, J=2.4 Hz, 1H), 8.30 (s, 1H), 8.17 (d, J=6.7 Hz, 1H), 8.08 (d, J=7.6 Hz, 1H), 7.91 (d, J=9.1 Hz, 2H), 7.82 (d, J=2.0 Hz, 1H), 7.35 (d, J=9.0 Hz, 2H), 6.69 (d, J=2.1 Hz, 1H), 4.49 (s, 2H), 3.83 (t, J=6.8 Hz, 2H), 3.76 (s, 2H), 3.66 (s, 2H), 3.48 (s, 5H), 3.40-3.27 (m, 2H), 2.86 (t, J=6.8 Hz, 2H), 2.78 (t, J=12.1 Hz, 2H), 2.05 (t, J=20.3 Hz, 2H), 1.79 (d, J=9.3 Hz, 2H). LCMS (ESI) calcd, for C₃₈H₃₈ClF₂N₁₀O₆⁺ [M+H]⁺: 803.26, found, 803.1.

### Example 266: Preparation of 2-((2-((4-(4-((2-(2,4-dioxotetrahydropyrimidin-1(2H)-yl)-1-oxoisoindolin-5-yl)methyl)piperazin-1-yl)-2-methoxyphenyl)amino)-5-(trifluoromethyl)pyridin-4-yl)amino)-N-methylbenzamide (GT-07660)

With reference to the method of Step 2 in Scheme T-1, the target compound (GT-07660) was obtained (yellow solid, 26 mg, yield 49.07%). ¹H NMR (400 MHz, DMSO-d₆) δ 11.91 (s, 1H), 10.70 (s, 1H), 10.59 (s, 1H), 10.00 (s, 1H), 8.82 (d, J=4.6 Hz, 1H), 8.14 (s, 1H), 7.98 (s, 1H), 7.88 (s, 2H), 7.77 (d, J=7.7 Hz, 1H), 7.56 (d, J=4.0 Hz, 2H), 7.34-7.24 (m, 1H), 7.18 (d, J=8.6 Hz, 1H), 4.85-4.50 (m, 4H), 3.89 (d, J=11.9 Hz, 2H), 3.81 (d, J=6.0 Hz, 5H), 3.40 (d, J=10.3 Hz, 2H), 3.28 (d, J=12.2 Hz, 2H), 3.20 (s, 2H), 2.79 (dd, J=26.6, 17.7 Hz, 5H). LCMS (ESI) calcd, for C₃₈H₃₉F₃N₉O₅⁺ [M+H]⁺: 758.30, found, 758.5.

### Example 267: Preparation of 2-((2-((4-(4-((2-(2,4-dioxotetrahydropyrimidin-1(2H)-yl)-1,3-dioxoisoindolin-5-yl)methyl)piperazin-1-yl)-2-methoxyphenyl)amino)-5-(trifluoromethyl)pyridin-4-yl)amino)-N-methylbenzamide (GT-07661)

With reference to the method of Step 2 in Scheme T-1, the target compound (GT-07661) was obtained (yellow solid, 20 mg, yield 37.06%). ¹H NMR (400 MHz, DMSO-d₆) δ 12.21-11.53 (m, 1H), 10.94 (s, 1H), 10.73-10.43 (m, 2H), 9.95 (s, 1H), 8.82 (d, J=4.6 Hz, 1H), 8.39 (s, 1H), 8.30-8.11 (m, 2H), 7.95 (s, 1H), 7.77 (d, J=7.8 Hz, 1H), 7.56 (t, J=6.2 Hz, 2H), 7.33-7.25 (m, 1H), 7.20 (d, J=8.6 Hz, 1H), 6.73 (s, 1H), 6.63-6.45 (m, 2H), 4.63 (s, 2H), 3.90-3.78 (m, 6H), 3.68 (d, J=5.9 Hz, 1H), 3.40 (s, 2H), 3.30-3.16 (m, 4H), 2.87 (t, J=6.7 Hz, 1H), 2.76 (t, J=5.2 Hz, 4H). LCMS (ESI) calcd, for C₃₈H₃₇F₃N₉O₆⁺ [M+H]⁺: 772.28, found, 772.4.

### Example 268: Preparation of 2-((2-((4-(4-(((2-(2,4-dioxotetrahydropyrimidin-1(2H)-yl)-1-oxoisoindolin-5-yl)methyl)(methyl)amino)piperidin-1-yl)-2-methoxyphenyl)amino)-5-(trifluoromethyl)pyridin-4-yl)amino)-N-methylbenzamide (GT-07647)

With reference to the method of Step 2 in Scheme T-1, the target compound (GT-07647) was obtained (yellow solid, 25 mg, yield 42.04%). ¹H NMR (500 MHz, DMSO) δ 11.44 (s, 1H), 10.65 (d, J=45.5 Hz, 2H), 10.01 (s, 1H), 8.84 (d, J=4.6 Hz, 1H), 8.17 (s, 1H), 8.01 (s, 1H), 7.87 (dd, J=16.5, 7.6 Hz, 2H), 7.78 (d, J=7.9 Hz, 1H), 7.57 (d, J=4.0 Hz, 2H), 7.32-7.21 (m, 2H), 6.96-6.40 (m, 3H), 4.93-4.73 (m, 1H), 4.61 (dd, J=19.0, 11.1 Hz, 2H), 4.39 (s, 1H), 3.91 (d, J=10.6 Hz, 2H), 3.84-3.78 (m, 5H), 3.48-3.45 (m, 2H), 2.88 (dd, J=17.0, 7.7 Hz, 3H), 2.76 (t, J=5.7 Hz, 4H), 2.62 (d, J=4.1 Hz, 2H), 2.36 (s, 1H), 2.27 (s, 1H), 2.07 (d, J=29.7 Hz, 2H). LCMS (ESI) calcd, for C₄₀H₄₃F₃N₉O₅⁺ [M+H]⁺: 786.33, found, 786.2.

### Example 269: Preparation of 2-((2-((4-(4-(((2-(2,4-dioxotetrahydropyrimidin-1(2H)-yl)-1,3-dioxoisoindolin-5-yl)methyl)(methyl)amino)piperidin-1-yl)-2-methoxyphenyl)amino)-5-(trifluoromethyl)pyridin-4-yl)amino)-N-methylbenzamide (GT-07648)

With reference to the method of Step 2 in Scheme T-1, the target compound (GT-07648) was obtained (yellow solid, 22 mg, yield 36.35%). ¹H NMR (500 MHz, DMSO) δ 11.43 (d, J=27.1 Hz, 1H), 10.94 (s, 1H), 10.59 (s, 1H), 9.95 (s, 1H), 8.82 (q, J=4.3 Hz, 1H), 8.42 (d, J=8.8 Hz, 1H), 8.30 (d, J=6.8 Hz, 1H), 8.16 (s, 1H), 8.10 (d, J=7.7 Hz, 1H), 7.78 (d, J=7.8 Hz, 1H), 7.61-7.55 (m, 2H), 7.35-7.19 (m, 2H), 6.83 (s, 1H), 6.71 (d, J=7.6 Hz, 1H), 6.56 (s, 1H), 4.72 (s, 1H), 4.49 (d, J=5.8 Hz, 1H), 4.02-3.92 (m, 2H), 3.83 (d, J=4.2 Hz, 5H), 3.66 (s, 1H), 2.86 (dd, J=13.4, 6.6 Hz, 4H), 2.76 (d, J=4.6 Hz, 3H), 2.63 (s, 3H), 2.33 (dd, J=25.9, 11.9 Hz, 2H), 1.99 (s, 2H). LCMS (ESI) calcd, for C₄₀H₄₁F₃N₉O₆⁺ [M+H]⁺: 800.31, found, 800.2.

### Example 270: Preparation of N-(3-(((2-((4-(4-((2-(2,4-dioxotetrahydropyrimidin-1(2H)-yl)-1-oxoisoindolin-5-yl)methyl)piperazin-1-yl)phenyl)amino)-5-(trifluoromethyl)pyrimidin-4-yl)amino)methyl)pyrazin-2-yl)-N-methylmethanesulfonamide (GT-07504)

With reference to the method of Step 2 in Scheme T-1, the target compound (GT-07504) was obtained (yellow solid, 18 mg, yield 30.44%). ¹H NMR (400 MHz, DMSO-d₆) δ 11.74 (s, 1H), 10.69 (s, 1H), 10.52 (s, 1H), 8.68 (d, J=2.5 Hz, 1H), 8.60 (d, J=2.4 Hz, 1H), 8.42 (s, 2H), 7.97 (s, 1H), 7.88 (d, J=7.9 Hz, 2H), 7.20 (s, 2H), 6.82 (d, J=7.6 Hz, 2H), 4.95 (d, J=5.1 Hz, 2H), 4.82 (d, J=16.8 Hz, 1H), 4.66-4.46 (m, 3H), 3.71 (s, 4H), 3.39 (s, 2H), 3.18 (s, 7H), 3.09 (s, 3H), 2.94-2.73 (m, 2H). LCMS (ESI) calcd, for C₃₅H₃₈F₃N₁₂O₅S⁺ [M+H]⁺: 795.28, found, 795.3.

### Example 271: Preparation of N-(3-(((2-((4-(4-((2-(2,4-dioxotetrahydropyrimidin-1(2H)-yl)-1,3-dioxoisoindolin-5-yl)methyl)piperazin-1-yl)phenyl)amino)-5-(trifluoromethyl)pyrimidin-4-yl)amino)methyl)pyrazin-2-yl)-N-methylmethanesulfonamide (GT-07505)

With reference to the method of Step 2 in Scheme T-1, the target compound (GT-07505) was obtained (yellow solid, 18 mg, yield 29.91%). ¹H NMR (400 MHz, DMSO-d₆) δ 11.84 (s, 1H), 10.95 (s, 1H), 10.57 (dd, J=57.3, 4.9 Hz, 1H), 8.69 (d, J=2.5 Hz, 1H), 8.61 (d, J=2.4 Hz, 1H), 8.38 (s, 2H), 8.24 (d, J=7.9 Hz, 1H), 8.12 (d, J=7.7 Hz, 1H), 7.23 (s, 2H), 6.84 (s, 2H), 4.96 (d, J=5.0 Hz, 2H), 4.62 (s, 2H), 3.84 (d, J=6.7 Hz, 2H), 3.69-3.66 (m, 2H), 3.41 (s, 2H), 3.18 (s, 7H), 3.10 (s, 3H), 2.87 (t, J=6.7 Hz, 2H). LCMS (ESI) calcd. for C₃₅H₃₆F₃N₁₂O₆S⁺ [M+H]⁺: 809.26, found, 809.3.

### Example 272: Preparation of N-(3-(((2-((4-(4-((1-(2-(2,4-dioxotetrahydropyrimidin-1(2H)-yl)-1,3-dioxoisoindolin-5-yl)piperidin-4-yl)methyl)piperazin-1-yl)phenyl)amino)-5-(trifluoromethyl)pyrimidin-4-yl)amino)methyl)pyrazin-2-yl)-N-methylmethanesulfonamide (GT-07468)

With reference to the method of Scheme T-3, the target compound (GT-07468) was obtained (yellow solid, 28 mg, yield 42.19%). ¹H NMR (500 MHz, DMSO) δ 10.95 (s, 1H), 10.84 (d, J=4.3 Hz, 1H), 8.69 (d, J=2.5 Hz, 1H), 8.62 (d, J=2.4 Hz, 1H), 8.50 (s, 1H), 7.71 (dd, J=14.5, 8.6 Hz, 1H), 7.42 (d, J=2.0 Hz, 1H), 7.38-7.30 (m, 1H), 7.19 (s, 2H), 6.87 (s, 2H), 4.96 (d, J=5.1 Hz, 2H), 4.12 (d, J=13.6 Hz, 4H), 3.79 (t, J=5.8 Hz, 2H), 3.70 (d, J=12.4 Hz, 2H), 3.61 (d, J=11.7 Hz, 2H), 3.33 (t, J=11.8 Hz, 2H), 3.20 (s, 3H), 3.06 (dd, J=22.3, 11.0 Hz, 7H), 2.86-2.81 (m, 2H), 2.24 (s, 1H), 1.99 (d, J=11.5 Hz, 2H), 1.30 (dd, J=24.4, 11.9 Hz, 2H) LCMS (ESI) calcd, for C₄₀H₄₅F₃N₁₃O₆S⁺ [M+H]⁺: 892.33, found, 892.4.

### Example 273: Preparation of N-(3-(((2-((4-(4-((1-(2-(2,4-dioxotetrahydropyrimidin-1(2H)-yl)-1,3-dioxoisoindolin-4-yl)piperidin-4-yl)methyl)piperazin-1-yl)phenyl)amino)-5-(trifluoromethyl)pyrimidin-4-yl)amino)methyl)pyrazin-2-yl)-N-methylmethanesulfonamide (GT-07469)

With reference to the method of Scheme T-3, the target compound (GT-07469) was obtained (yellow solid, 18 mg, yield 27.12%). ¹H NMR (500 MHz, DMSO) δ 10.86 (s, 1H), 10.77 (s, 1H), 10.48 (s, 1H), 8.70 (d, J=2.5 Hz, 1H), 8.62 (d, J=2.4 Hz, 1H), 8.40 (d, J=31.6 Hz, 2H), 7.75 (dd, J=15.1, 7.8 Hz, 1H), 7.41 (dd, J=11.3, 7.8 Hz, 2H), 7.24 (s, 2H), 6.88 (s, 2H), 4.97 (d, J=5.1 Hz, 2H), 3.90-3.78 (m, 6H), 3.65-3.60 (m, 7H), 3.31 (s, 2H), 3.12 (s, 5H), 2.96 (dt, J=22.7, 11.4 Hz, 2H), 2.84 (d, J=6.0 Hz, 2H), 2.13 (s, 1H), 2.03 (d, J=8.7 Hz, 2H), 1.56-1.40 (m, 2H). LCMS (ESI) calcd, for C₄₀H₄₅F₃N₁₃O₆S⁺ [M+H]⁺: 892.33, found, 892.4.

### Example 274: Preparation of 1-(5-((4-(4-((4-((3-(methylsulfonyl)benzyl)amino)-5-(trifluoromethyl)pyrimidin-2-yl)amino)phenyl)piperazin-1-yl)methyl)-1-oxoisoindolin-2-yl)dihydropyrimidine-2,4 (1H,3H)-dione (GT-07514)

With reference to the method of Step 2 in Scheme T-1, the target compound (GT-07514) was obtained (yellow solid, 14 mg, yield 23.21%). ¹H NMR (500 MHz, DMSO) δ 11.67 (s, 1H), 10.66 (d, J=35.4 Hz, 2H), 8.93 (s, 1H), 8.45 (d, J=15.4 Hz, 1H), 7.96 (s, 1H), 7.93-7.84 (m, 3H), 7.81 (d, J=7.5 Hz, 1H), 7.61 (t, J=7.7 Hz, 1H), 7.57-7.48 (m, 1H), 7.27 (d, J=6.4 Hz, 2H), 6.91 (d, J=8.3 Hz, 2H), 4.82 (d, J=16.6 Hz, 1H), 4.72 (d, J=5.3 Hz, 2H), 4.63-4.50 (m, 3H), 3.77 (d, J=11.0 Hz, 4H), 3.39 (s, 2H), 3.23-3.12 (m, 7H), 2.93-2.71 (m, 2H). LCMS (ESI) calcd, for C₃₆H₃₇F₃N₉O₅S⁺ [M+H]⁺: 764.26, found, 764.3.

### Example 275: Preparation of 2-(2,4-dioxotetrahydropyrimidin-1(2H)-yl)-5-((4-(4-((4-((3-(methylsulfonyl)benzyl)amino)-5-(trifluoromethyl)pyrimidin-2-yl)amino)phenyl)piperazin-1-yl)methyl)isoindoline-1,3-dione (GT-07515)

With reference to the method of Step 2 in Scheme T-1, the target compound (GT-07515) was obtained (yellow solid, mg, yield %). ¹H NMR (500 MHz, DMSO) δ 11.71 (s, 1H), 10.93 (s, 1H), 10.57-10.23 (m, 1H), 8.75 (s, 1H), 8.36 (s, 2H), 8.23 (d, J=7.8 Hz, 1H), 8.12 (d, J=7.7 Hz, 1H), 7.90 (s, 1H), 7.80 (d, J=7.4 Hz, 1H), 7.60 (d, J=7.7 Hz, 2H), 7.28 (s, 2H), 6.90 (d, J=8.2 Hz, 2H), 4.71 (d, J=5.4 Hz, 2H), 4.62 (s, 2H), 3.83 (s, 2H), 3.40 (s, 2H), 3.24-3.12 (m, 7H), 2.86 (t, J=6.8 Hz, 2H). LCMS (ESI) calcd, for C₃₆H₃₅F₃N₉O₆S⁺ [M+H]⁺: 778.24, found, 778.3.

### Example 276: Preparation of 2-(2,4-dioxotetrahydropyrimidin-1(2H)-yl)-5-(4-((4-(4-((4-((3-(methylsulfonyl)benzyl)amino)-5-(trifluoromethyl)pyrimidin-2-yl)amino)phenyl)piperazin-1-yl)methyl)piperidin-1-yl)isoindoline-1,3-dione (GT-07470)

With reference to the method of Scheme T-3, the target compound (GT-07470) was obtained (yellow solid, 23 mg, yield 33.83%). ¹H NMR (500 MHz, DMSO) δ 10.84 (d, J=4.8 Hz, 1H), 10.75 (s, 2H), 8.99 (s, 1H), 8.43 (t, J=28.7 Hz, 1H), 7.92 (s, 1H), 7.83 (d, J=7.4 Hz, 1H), 7.72 (dd, J=15.9, 8.6 Hz, 1H), 7.62 (t, J=7.7 Hz, 1H), 7.54 (s, 1H), 7.42 (d, J=2.0 Hz, 1H), 7.38-7.26 (m, 3H), 6.96 (d, J=8.2 Hz, 2H), 4.73 (d, J=5.4 Hz, 2H), 4.11 (s, 2H), 3.78 (dd, J=6.7, 4.8 Hz, 5H), 3.61 (d, J=11.2 Hz, 2H), 3.34-3.27 (m, 2H), 3.17 (s, 4H), 3.07 (d, J=12.7 Hz, 3H), 2.84 (td, J=6.6, 2.2 Hz, 2H), 2.25 (s, 1H), 1.98 (d, J=11.2 Hz, 2H), 1.79-1.66 (m, 1H), 1.30 (d, J=12.3 Hz, 2H). LCMS (ESI) calcd, for C₄₁H₄₄F₃N₁₀O₆S⁺ [M+H]⁺: 861.31, found, 861.3.

### Example 277: Preparation of 2-(2,4-dioxotetrahydropyrimidin-1(2H)-yl)-4-(4-((4-(4-((4-((3-(methylsulfonyl)benzyl)amino)-5-(trifluoromethyl)pyrimidin-2-yl)amino)phenyl)piperazin-1-yl)methyl)piperidin-1-yl)isoindoline-1,3-dione (GT-07471)

With reference to the method of Scheme T-3, the target compound (GT-07471) was obtained (yellow solid, 12 mg, yield 17.65%). ¹H NMR (500 MHz, DMSO) δ 10.85 (d, J=3.1 Hz, 3H), 9.16 (d, J=73.2 Hz, 1H), 8.48 (s, 1H), 7.91 (s, 1H), 7.82 (d, J=7.4 Hz, 1H), 7.75 (dd, J=8.4, 7.3 Hz, 1H), 7.62 (t, J=7.7 Hz, 1H), 7.54 (s, 1H), 7.40 (dd, J=12.2, 7.9 Hz, 2H), 7.28 (d, J=7.9 Hz, 2H), 6.96 (d, J=8.2 Hz, 2H), 4.73 (d, J=5.3 Hz, 2H), 3.80-3.75 (m, 4H), 3.69-3.60 (m, 3H), 3.35 (dd, J=21.7, 9.1 Hz, 2H), 3.21-3.12 (m, 7H), 3.03-2.91 (m, 2H), 2.82 (dd, J=6.6, 3.9 Hz, 2H), 2.13 (s, 1H), 2.03 (d, J=9.4 Hz, 2H), 1.96-1.58 (m, 1H), 1.54-1.38 (m, 2H). LCMS (ESI) calcd, for C₄₁H₄₄F₃N₁₀O₆S⁺ [M+H]⁺: 861.31, found, 861.3.

### Example 278: Preparation of 2-((2-((4-(1-((2-(2,4-dioxotetrahydropyrimidin-1(2H)-yl)-1-oxoisoindolin-5-yl)methyl)piperidin-4-yl)-2-isopropoxy-5-methylphenyl)amino)-5-(trifluoromethyl)pyridin-4-yl)amino)-N-methylbenzamide (GT-07663)

With reference to the method of Step 2 in Scheme T-1, the target compound (GT-07663) was obtained (yellow solid, 24 mg, yield 40.68%). ¹H NMR (400 MHz, DMSO-d₆) δ 11.38 (s, 1H), 10.69 (d, J=10.9 Hz, 1H), 10.58 (s, 1H), 9.66 (s, 1H), 8.78 (s, 1H), 8.27 (d, J=20.3 Hz, 1H), 7.96 (s, 1H), 7.87 (q, J=7.9 Hz, 2H), 7.77 (t, J=9.2 Hz, 1H), 7.67-7.59 (m, 1H), 7.52 (t, J=7.7 Hz, 1H), 7.24 (dd, J=14.3, 6.4 Hz, 2H), 6.91 (s, 1H), 6.66 (d, J=19.7 Hz, 1H), 4.81 (t, J=18.1 Hz, 1H), 4.59 (d, J=16.5 Hz, 1H), 4.53-4.41 (m, 3H), 3.80 (t, J=6.8 Hz, 2H), 3.37-3.25 (m, 2H), 3.09 (d, J=10.3 Hz, 2H), 2.98 (t, J=12.0 Hz, 1H), 2.90-2.83 (m, 1H), 2.76 (t, J=7.3 Hz, 4H), 2.30-2.16 (m, 5H), 1.81 (t, J=17.5 Hz, 2H), 1.22 (dd, J=16.5, 6.0 Hz, 6H). LCMS (ESI) calcd. for C₄₂H₄₆F₃N₈O₅⁺ [M+H]⁺: 799.35, found, 799.5.

### Example 279: Preparation of 2-((2-((4-(1-((2-(2,4-dioxotetrahydropyrimidin-1(2H)-yl)-1,3-dioxoisoindolin-5-yl)methyl)piperidin-4-yl)-2-isopropoxy-5-methylphenyl)amino)-5-(trifluoromethyl)pyridin-4-yl)amino)-N-methylbenzamide (GT-07664)

With reference to the method of Step 2 in Scheme T-1, the target compound (GT-07664) was obtained (yellow solid, 28 mg, yield 46.64%). ¹H NMR (400 MHz, DMSO-d₆) δ 11.69 (s, 1H), 11.24 (d, J=77.1 Hz, 1H), 10.93 (s, 1H), 10.61 (d, J=13.9 Hz, 1H), 9.72 (s, 1H), 8.79 (d, J=3.9 Hz, 1H), 8.39 (s, 1H), 8.25 (d, J=6.8 Hz, 2H), 8.12 (t, J=8.7 Hz, 1H), 8.02-7.88 (m, 1H), 7.75 (t, J=7.3 Hz, 1H), 7.65-7.46 (m, 2H), 7.23 (dd, J=15.1, 7.5 Hz, 2H), 6.91 (s, 1H), 6.63 (s, 1H), 4.57 (s, 1H), 4.47 (dt, J=12.0, 6.0 Hz, 2H), 3.83 (t, J=6.7 Hz, 2H), 3.71-3.66 (m, 1H), 3.41-3.21 (m, 3H), 3.10 (d, J=10.4 Hz, 2H), 2.98-2.84 (m, 2H), 2.75 (d, J=4.5 Hz, 4H), 2.25 (d, J=11.9 Hz, 5H), 1.83 (d, J=12.5 Hz, 2H), 1.20 (t, J=8.9 Hz, 6H). LCMS (ESI) calcd. for C₄₂H₄₄F₃N₈O₆⁺ [M+H]⁺: 813.33, found, 813.4.

### Example 280: Preparation of 2-((2-((4-(1-((1-(2-(2,4-dioxotetrahydropyrimidin-1(2H)-yl)-1,3-dioxoisoindolin-5-yl)piperidin-4-yl)methyl)piperidin-4-yl)-2-isopropoxy-5-methylphenyl)amino)-5-(trifluoromethyl)pyridin-4-yl)amino)-N-methylbenzamide (GT-07665)

With reference to the method of Scheme T-3, the target compound (GT-07665) was obtained (yellow solid, 27 mg, yield 40.80%). ¹H NMR (400 MHz, DMSO-d₆) δ 10.85 (s, 1H), 10.66 (s, 2H), 9.95 (s, 1H), 8.83 (s, 1H), 8.29 (s, 1H), 7.79 (d, J=7.8 Hz, 1H), 7.73 (d, J=8.6 Hz, 1H), 7.63 (d, J=7.9 Hz, 1H), 7.55 (s, 1H), 7.42 (s, 1H), 7.31 (dd, J=12.8, 9.0 Hz, 2H), 7.19 (s, 1H), 7.02 (s, 1H), 6.64 (s, 1H), 4.52 (s, 1H), 4.13 (d, J=13.5 Hz, 2H), 3.79 (dd, J=8.3, 5.3 Hz, 3H), 3.29 (m, 2H), 3.03 (dd, J=21.1, 8.5 Hz, 6H), 2.84 (t, J=7.2 Hz, 2H), 2.76 (d, J=4.5 Hz, 3H), 2.39 (d, J=12.6 Hz, 2H), 2.27 (s, 4H), 2.02 (d, J=11.7 Hz, 2H), 1.81 (d, J=12.0 Hz, 2H), 1.33 (t, J=17.2 Hz, 2H), 1.21 (t, J=7.0 Hz, 6H). LCMS (ESI) calcd, for C₄₇H₅₃F₃N₉O₆⁺ [M+H]⁺: 896.41, found, 896.4.

### Example 281: Preparation of 2-((2-((4-(1-((1-(2-(2,4-dioxotetrahydropyrimidin-1(2H)-yl)-1,3-dioxoisoindolin-4-yl)piperidin-4-yl)methyl)piperidin-4-yl)-2-isopropoxy-5-methylphenyl)amino)-5-(trifluoromethyl)pyridin-4-yl)amino)-N-methylbenzamide (GT-07666)

With reference to the method of Scheme T-3, the target compound (GT-07666) was obtained (yellow solid, 22 mg, yield 33.25%). ¹H NMR (400 MHz, DMSO-d₆) δ 10.86 (s, 1H), 10.64 (s, 1H), 10.52 (s, 1H), 9.84 (s, 1H), 8.81 (s, 1H), 8.28 (s, 1H), 7.83-7.73 (m, 2H), 7.62 (s, 1H), 7.55 (s, 1H), 7.41 (t, J=8.5 Hz, 2H), 7.28 (s, 1H), 7.21 (s, 1H), 7.02 (s, 1H), 6.64 (s, 1H), 4.52 (dt, J=12.0, 6.0 Hz, 1H), 3.88-3.76 (m, 4H), 3.63 (s, 3H), 3.34 (d, J=20.6 Hz, 1H), 3.06 (s, 5H), 2.88-2.81 (m, 2H), 2.76 (d, J=4.6 Hz, 3H), 2.40 (d, J=11.0 Hz, 2H), 2.27 (s, 3H), 2.08 (d, J=11.6 Hz, 3H), 1.82 (d, J=13.2 Hz, 2H), 1.48 (dd, J=19.5, 9.5 Hz, 2H), 1.21 (t, J=5.5 Hz, 6H). LCMS (ESI) calcd, for C₄₇H₅₃F₃N₉O₆⁺ [M+H]⁺: 896.41, found, 896.5.

### Example 282: Preparation of N-(3-(((2-((4-(4-(((2-(2,4-dioxotetrahydropyrimidin-1(2H)-yl)-3-oxoisoindolin-5-yl)methyl)(methyl)amino)piperidin-1-yl)phenyl)amino)-5-(trifluoromethyl)pyrimidin-4-yl)amino)methyl)pyrazin-2-yl)-N-methylmethanesulfonamide (GT-08136)

With reference to the method of Scheme T-3, the target compound (GT-08136) was obtained (white solid, 31 mg, yield 55%). ¹H NMR (400 MHz, DMSO-d₆) δ 11.41 (s, 1H), 10.68 (s, 1H), 10.30 (s, 1H), 8.65 (dd, J=36.1, 2.4 Hz, 2H), 8.39 (s, 1H), 8.00 (s, 2H), 7.91-7.84 (m, 2H), 7.47 (s, 2H), 7.31 (s, 2H), 5.00 (d, J=5.0 Hz, 2H), 4.88-4.78 (m, 1H), 4.65-4.53 (m, 2H), 4.45-4.38 (m, 1H), 3.80 (t, 4H), 3.21 (s, 3H), 3.18 (s, 3H), 2.90-2.84 (m, 1H), 2.80-2.75 (m, 1H), 2.65 (s, 3H), 2.37 (d, J=29.7 Hz, 4H). LCMS (ESI) calcd. for C₃₇H₄₂F₃N₁₂O₅S⁺ [M+H]⁺: 823.31, found, 823.2.

### Example 283: Preparation of N-(3-(((2-((4-(4-(((2-(2,4-dioxotetrahydropyrimidin-1(2H)-yl)-1,3-dioxoisoindolin-5-yl)methyl)(methyl)amino)piperidin-1-yl)phenyl)amino)-5-(trifluoromethyl)pyrimidin-4-yl)amino)methyl)pyrazin-2-yl)-N-methylmethanesulfonamide (GT-08137)

With reference to the method of Scheme T-3, the target compound (GT-08137) was obtained (white solid, 33 mg, yield 58%). ¹H NMR (400 MHz, DMSO-d₆) δ 11.51 (s, 1H), 10.92 (s, 1H), 10.23 (s, 1H), 8.69 (d, J=2.5 Hz, 1H), 8.60 (d, J=2.4 Hz, 1H), 8.45-8.36 (m, 2H), 8.33-8.26 (m, 1H), 8.11 (d, J=7.7 Hz, 1H), 7.99-7.88 (m, 1H), 7.47 (s, 2H), 7.26 (s, 2H), 4.99 (d, J=5.0 Hz, 2H), 4.69 (s, 1H), 4.52 (s, 1H), 3.83 (t, J=6.7 Hz, 4H), 3.20 (s, 3H), 3.18 (s, 3H), 2.86 (t, J=6.7 Hz, 2H), 2.64 (s, 3H), 2.43-2.31 (m, 3H), 2.30-2.23 (m, 1H). LCMS (ESI) calcd, for C₃₇H₄₀F₃N₁₂O₆S⁺ [M+H]⁺: 837.29, found, 837.1.

### Example 284: Preparation of N-(3-(((2-((4-(4-(4-((2-(2,4-dioxotetrahydropyrimidin-1(2H)-yl)-1-oxoisoindolin-5-yl)methyl)piperazin-1-yl)piperidin-1-yl)phenyl)amino)-5-(trifluoromethyl)pyrimidin-4-yl)amino)methyl)pyrazin-2-yl)-N-methylmethanesulfonamide (GT-08138)

With reference to the method of Scheme T-3, the target compound (GT-08138) was obtained (white solid, 31 mg, yield 56%). ¹H NMR (400 MHz, DMSO-d₆) δ 10.76-10.61 (m, 1H), 10.19 (s, 1H), 8.69 (d, J=2.4 Hz, 1H), 8.60 (d, J=2.3 Hz, 1H), 8.36 (s, 1H), 7.91 (s, 2H), 7.85 (q, J=7.9 Hz, 2H), 7.42 (s, 2H), 7.17 (s, 2H), 4.98 (d, J=4.9 Hz, 2H), 4.81 (d, J=16.4 Hz, 1H), 4.61-4.48 (m, 3H), 3.81-3.77 (m, 4H), 3.72 (s, 4H), 3.48-3.41 (m, 4H), 3.18 (d, J=12.3 Hz, 6H), 3.02 (s, 2H), 2.95-2.81 (m, 2H), 2.80-2.73 (m, 1H), 2.24 (s, 2H), 2.14-1.97 (m, 2H). LCMS (ESI) calcd, for C₄₀H₄₇F₃N₁₃O₅S⁺ [M+H]⁺: 878.35, found, 878.1.

### Example 285: Preparation of N-(3-(((2-((4-(4-(4-((2-(2,4-dioxotetrahydropyrimidin-1(2H)-yl)-1,3-dioxoisoindolin-5-yl)methyl)piperazin-1-yl)piperidin-1-yl)phenyl)amino)-5-(trifluoromethyl)pyrimidin-4-yl)amino)methyl)pyrazin-2-yl)-N-methylmethanesulfonamide (GT-08139)

With reference to the method of Scheme T-3, the target compound (GT-08139) was obtained (white solid, 29 mg, yield 47%). ¹H NMR (400 MHz, DMSO-d₆) δ 10.93 (s, 1H), 10.21 (s, 1H), 8.69 (d, J=2.4 Hz, 1H), 8.60 (d, J=2.3 Hz, 1H), 8.34 (d, J=16.4 Hz, 2H), 8.19 (d, J=7.5 Hz, 1H), 8.09 (d, J=7.6 Hz, 1H), 8.02-7.89 (m, 1H), 7.42 (s, 2H), 7.18 (s, 2H), 4.98 (d, J=4.9 Hz, 2H), 4.53 (s, 2H), 3.86-3.79 (m, 5H), 3.76-3.71 (m, 6H), 3.54-3.51 (m, 3H), 3.20 (s, 3H), 3.17 (s, 3H), 3.06 (s, 2H), 2.86 (t, J=6.7 Hz, 2H), 2.32-2.23 (m, 2H), 2.11-2.05 (m, 1H). LCMS (ESI) calcd, for C₄₀H₄₅F₃N₁₃O₆S⁺ [M+H]⁺: 892.33, found, 892.1.

### Example 286: Preparation of N-(3-(((2-((4-(4-((2-(2,4-dioxotetrahydropyrimidin-1(2H)-yl)-1-oxoisoindolin-5-yl)methyl)piperazin-1-yl)phenyl)amino)-5-(trifluoromethyl)pyrimidin-4-yl)amino)methyl)phenyl)-N-methylmethanesulfonamide (GT-08140)

With reference to the method of Scheme T-3, the target compound (GT-08140) was obtained (white solid, 32 mg, yield 56%). ¹H NMR (400 MHz, DMSO-d₆) δ 11.58 (s, 1H), 10.68 (s, 1H), 10.51 (s, 1H), 8.80 (s, 1H), 8.38 (s, 1H), 7.96 (s, 1H), 7.91-7.83 (m, 2H), 7.40-7.30 (m, 4H), 7.28 (d, J=7.8 Hz, 1H), 7.19 (s, 1H), 6.93 (d, J=8.6 Hz, 2H), 4.82 (d, J=16.1 Hz, 1H), 4.65-4.60 (m, 2H), 4.53 (s, 2H), 3.82-3.77 (m, 6H), 3.40 (s, 2H), 3.20 (s, 3H), 3.15 (s, 3H), 2.87 (s, 4H), 2.81-2.74 (m, 1H). LCMS (ESI) calcd, for C₃₇H₄₀F₃N₁₀O₅S⁺ [M+H]⁺: 793.29, found, 793.1.

### Example 287: Preparation of N-(3-(((2-((4-(4-((2-(2,4-dioxotetrahydropyrimidin-1(2H)-yl)-1,3-dioxoisoindolin-5-yl)methyl)piperazin-1-yl)phenyl)amino)-5-(trifluoromethyl)pyrimidin-4-yl)amino)methyl)phenyl)-N-methylmethanesulfonamide (GT-08141)

With reference to the method of Scheme T-3, the target compound (GT-08141) was obtained (white solid, 29 mg, yield 50%). ¹H NMR (400 MHz, DMSO-d₆) δ 11.79 (s, 1H), 10.94 (s, 1H), 10.49 (s, 1H), 8.79 (s, 1H), 8.37 (s, 2H), 8.24 (d, J=7.3 Hz, 1H), 8.12 (d, J=7.7 Hz, 1H), 7.41-7.31 (m, 5H), 7.28 (d, J=7.9 Hz, 1H), 7.19 (s, 1H), 6.93 (d, J=8.7 Hz, 2H), 4.63 (s, 4H), 3.84-3.80 (m, 6H), 3.41 (s, 2H), 3.20 (s, 3H), 3.15 (s, 3H), 2.88-2.86 (m, 4H). LCMS (ESI) calcd, for C₃₇H₃₈F₃N₁₀O₆S⁺ [M+H]⁺: 807.26, found, 807.1.

### Example 288: Preparation of N-(3-(((2-((4-(4-(((2-(2,4-dioxotetrahydropyrimidin-1(2H)-yl)-1-oxoisoindolin-5-yl)methyl)(methyl)amino)piperidin-1-yl)phenyl)amino)-5-(trifluoromethyl)pyrimidin-4-yl)amino)methyl)phenyl)-N-methylmethanesulfonamide (GT-08142)

With reference to the method of Scheme T-3, the target compound (GT-08142) was obtained (white solid, 19 mg, yield 34%). ¹H NMR (400 MHz, DMSO-d₆) δ 11.31 (s, 1H), 10.70 (s, 1H), 10.31 (s, 1H), 8.44 (s, 1H), 8.35 (s, 1H), 7.99 (s, 1H), 7.88 (s, 2H), 7.49 (s, 2H), 7.41-7.37 (m, 1H), 7.35 (s, 1H), 7.28 (d, J=8.0 Hz, 2H), 7.22 (d, J=7.2 Hz, 1H), 4.86-4.79 (m, 1H), 4.67-4.59 (m, 4H), 4.45-4.38 (m, 1H), 3.80 (s, 6H), 3.55-3.51 (m, 2H), 3.16 (s, 3H), 2.88 (s, 4H), 2.80-2.76 (m, 1H), 2.67-2.63 (m, 3H), 2.42-2.32 (m, 3H). LCMS (ESI) calcd, for C₃₉H₄₄F₃N₁₀O₅S⁺ [M+H]⁺: 821.32, found, 821.2.

### Example 289: Preparation of N-(3-(((2-((4-(4-(((2-(2,4-dioxotetrahydropyrimidin-1(2H)-yl)-1,3-dioxoisoindolin-5-yl)methyl)(methyl)amino)piperidin-1-yl)phenyl)amino)-5-(trifluoromethyl)pyrimidin-4-yl)amino)methyl)phenyl)-N-methylmethanesulfonamide (GT-08143)

With reference to the method of Scheme T-3, the target compound (GT-08143) was obtained (white solid, 13 mg, yield 23%). ¹H NMR (400 MHz, DMSO-d₆) δ 11.43 (s, 1H), 10.92 (s, 1H), 10.38 (s, 1H), 8.42 (d, J=12.8 Hz, 1H), 8.35-8.25 (m, 2H), 8.11 (d, J=7.7 Hz, 1H), 7.49 (s, 2H), 7.41-7.34 (m, 2H), 7.31-7.25 (m, 2H), 7.22 (d, J=7.3 Hz, 1H), 4.74-4.63 (m, 3H), 4.52 (s, 1H), 3.85-3.79 (m, 6H), 3.56-3.52 (m, 2H), 3.16 (s, 3H), 2.88 (s, 3H), 2.85 (d, J=6.6 Hz, 2H), 2.64 (s, 3H), 2.41-2.33 (m, 2H), 2.27-2.19 (m, 1H). LCMS (ESI) calcd, for C₃₉H₄₂F₃N₁₀O₆S⁺ [M+H]⁺: 835.30, found, 835.1.

### Example 290: Preparation of N-(3-(((2-((4-(4-(4-((2-(2,4-dioxotetrahydropyrimidin-1(2H)-yl)-1-oxoisoindolin-5-yl)methyl)piperazin-1-yl)piperidin-1-yl)phenyl)amino)-5-(trifluoromethyl)pyrimidin-4-yl)amino)methyl)phenyl)-N-methylmethanesulfonamide (GT-08144)

With reference to the method of Scheme T-3, the target compound (GT-08144) was obtained (white solid, 29 mg, yield 53%). ¹H NMR (400 MHz, DMSO-d₆) δ 11.95 (s, 1H), 10.68 (s, 1H), 10.24 (s, 1H), 8.49 (s, 1H), 8.33 (s, 1H), 7.92 (s, 1H), 7.85 (t, J=8.4 Hz, 2H), 7.45 (s, 2H), 7.39-7.36 (m, 1H), 7.35 (s, 1H), 7.28 (d, J=7.7 Hz, 1H), 7.21 (d, J=7.1 Hz, 2H), 4.83-4.77 (m, 1H), 4.65 (d, J=5.3 Hz, 2H), 4.57 (d, J=16.8 Hz, 2H), 4.51 (s, 1H), 3.82-3.76 (m, 8H), 3.48-3.40 (m, 4H), 3.16 (s, 3H), 3.04-2.98 (m, 1H), 2.87 (s, 4H), 2.80-2.76 (m, 1H), 2.23 (s, 2H), 2.04 (s, 2H). LCMS (ESI) calcd, for C₄₂H₄₉F₃N₁₁O₅S⁺ [M+H]⁺: 876.36, found, 876.2.

### Example 291: Preparation of N-(3-(((2-((4-(4-(4-((2-(2,4-dioxotetrahydropyrimidin-1(2H)-yl)-1,3-dioxoisoindolin-5-yl)methyl)piperazin-1-yl)piperidin-1-yl)phenyl)amino)-5-(trifluoromethyl)pyrimidin-4-yl)amino)methyl)phenyl)-N-methylmethanesulfonamide (GT-08145)

With reference to the method of Scheme T-3, the target compound (GT-08145) was obtained (white solid, 30 mg, yield 54%). ¹H NMR (400 MHz, DMSO-d₆) δ 12.04 (s, 1H), 10.93 (s, 1H), 10.34 (s, 1H), 8.58 (s, 1H), 8.33 (s, 2H), 8.20 (d, J=7.5 Hz, 1H), 8.10 (d, J=7.7 Hz, 1H), 7.46 (s, 2H), 7.39 (t, J=7.8 Hz, 1H), 7.35 (s, 1H), 7.29 (d, J=7.9 Hz, 2H), 7.22 (d, J=7.5 Hz, 2H), 4.66 (d, J=5.2 Hz, 2H), 4.54 (s, 2H), 3.85-3.81 (m, 6H), 3.77 (s, 3H), 3.54 (s, 4H), 3.16 (s, 3H), 3.08-3.03 (m, 1H), 2.90-2.84 (m, 6H), 2.31-2.24 (m, 2H), 2.14-2.03 (m, 2H). LCMS (ESI) calcd, for C₄₂H₄₇F₃N₁₁O₆S⁺ [M+H]⁺: 890.34, found, 890.1.

### Example 292: Preparation of N-(3-(((2-((4-(4-(4-((1-(2-(2,4-dioxotetrahydropyrimidin-1(2H)-yl)-1,3-dioxoisoindolin-4-yl)piperidin-4-yl)methyl)piperazin-1-yl)piperidin-1-yl)phenyl)amino)-5-(trifluoromethyl)pyrimidin-4-yl)amino)methyl)pyrazin-2-yl)-N-methylmethanesulfonamide (GT-08245)

With reference to the method of Scheme T-3, the target compound (GT-08245) was obtained (yellow solid, 33 mg, yield 52%). ¹H NMR (400 MHz, DMSO-d₆) δ 11.56 (s, 1H), 10.85 (s, 1H), 10.10 (s, 1H), 8.70 (d, J=2.5 Hz, 1H), 8.61 (d, J=2.3 Hz, 1H), 8.35 (s, 1H), 7.87 (s, 1H), 7.75 (t, 1H), 7.51-7.31 (m, 4H), 7.14 (s, 2H), 4.99 (d, J=5.0 Hz, 2H), 3.77-3.74 (m, 6H), 3.69-3.68 (m, 4H), 3.56-3.49 (m, 4H), 3.19 (d, J=10.8 Hz, 6H), 3.08-2.87 (m, 4H), 2.83 (t, J=6.8 Hz, 2H), 2.34-2.21 (m, 2H), 2.07 (s, 5H), 1.53-1.34 (m, 2H). LCMS (ESI) calcd, for C₄₅H₅₄F₃N₁₄O₆S⁺ [M+H]⁺: 975.40, found, 975.1.

### Example 293: Preparation of N-(3-(((2-((4-(4-((1-(2-(2,4-dioxotetrahydropyrimidin-1(2H)-yl)-1,3-dioxoisoindolin-5-yl)piperidin-4-yl)methyl)piperazin-1-yl)phenyl)amino)-5-(trifluoromethyl)pyrimidin-4-yl)amino)methyl)phenyl)-N-methylmethanesulfonamide (GT-08246)

With reference to the method of Scheme T-3, the target compound (GT-08246) was obtained (yellow solid, 15 mg, yield 23%). ¹H NMR (400 MHz, DMSO-d₆) δ 10.84 (s, 1H), 10.46 (s, 1H), 8.33 (s, 1H), 7.74-7.68 (m, 1H), 7.43-7.38 (m, 2H), 7.38-7.34 (m, 3H), 7.34-7.24 (m, 3H), 7.21 (s, 1H), 6.95 (d, J=8.6 Hz, 2H), 4.64 (d, J=5.3 Hz, 2H), 4.11 (t, J=14.8 Hz, 3H), 3.80-3.77 (m, 4H), 3.76-3.71 (m, 4H), 3.60 (d, J=10.3 Hz, 2H), 3.29-3.24 (m, 2H), 3.16 (s, 4H), 2.88 (s, 3H), 2.22 (s, 1H), 1.95 (d, J=11.3 Hz, 2H), 1.74 (d, J=13.2 Hz, 1H), 1.36-1.24 (m, 2H), 1.22-1.10 (m, 1H). LCMS (ESI) calcd, for C₄₂H₄₇F₃N₁₁O₆S⁺ [M+H]⁺: 890.34, found, 890.1.

### Example 294: Preparation of N-(3-(((2-((4-(4-((1-(2-(2,4-dioxotetrahydropyrimidin-1(2H)-yl)-1,3-dioxoisoindolin-4-yl)piperidin-4-yl)methyl)piperazin-1-yl)phenyl)amino)-5-(trifluoromethyl)pyrimidin-4-yl)amino)methyl)phenyl)-N-methylmethanesulfonamide (GT-08247)

With reference to the method of Scheme T-3, the target compound (GT-08247) was obtained (yellow solid, 10 mg, yield 15%). ¹H NMR (400 MHz, DMSO-d₆) δ 10.86 (s, 2H), 10.59 (s, 1H), 8.38 (s, 1H), 7.82-7.63 (m, 2H), 7.40-7.35 (m, 5H), 7.29 (d, J=8.3 Hz, 1H), 7.20 (s, 1H), 6.97 (d, J=8.6 Hz, 2H), 4.64 (d, J=5.2 Hz, 2H), 3.81-3.75 (m, 6H), 3.61 (d, 2H), 3.32 (d, J=6.1 Hz, 2H), 3.27 (d, J=11.8 Hz, 1H), 3.16 (s, 6H), 2.99-2.92 (m, 2H), 2.89 (s, 3H), 2.85-2.81 (m, 3H), 2.12 (s, 1H), 2.01 (s, 2H), 1.79-1.74 (m, 1H), 1.51-1.30 (m, 3H). LCMS (ESI) calcd, for C₄₂H₄₇F₃N₁₁O₆S⁺ [M+H]⁺: 890.34, found, 890.2.

### Example 295: Preparation of N-(3-(((2-((4-(4-(4-((1-(2-(2,4-dioxotetrahydropyrimidin-1(2H)-yl)-1,3-dioxoisoindolin-4-yl)piperidin-4-yl)methyl)piperazin-1-yl)piperidin-1-yl)phenyl)amino)-5-(trifluoromethyl)pyrimidin-4-yl)amino)methyl)phenyl)-N-methylmethanesulfonamide (GT-08248)

With reference to the method of Scheme T-3, the target compound (GT-08248) was obtained (yellow solid, 17 mg, yield 28%). ¹H NMR (400 MHz, DMSO-d₆) δ 11.68 (s, 1H), 10.85 (s, 1H), 10.42 (s, 1H), 8.53 (s, 1H), 8.37 (s, 1H), 7.78-7.72 (m, 1H), 7.50-7.45 (m, 2H), 7.44-7.37 (m, 4H), 7.35 (s, 1H), 7.29 (d, J=7.7 Hz, 2H), 7.22 (d, J=7.4 Hz, 1H), 4.66 (d, J=5.3 Hz, 2H), 3.92-3.85 (m, 4H), 3.83-3.70 (m, 10H), 3.56 (s, 4H), 3.16 (s, 3H), 3.00 (s, 1H), 2.98-2.91 (m, 2H), 2.88 (s, 3H), 2.83 (t, J=6.8 Hz, 2H), 2.31 (d, J=11.1 Hz, 2H), 2.18-2.05 (m, 5H), 1.51-1.37 (m, 2H). LCMS (ESI) calcd, for C₄₇H₅₆F₃N₁₂O₆S⁺ [M+H]⁺: 973.41, found, 973.2.

### Example 296: Preparation of 4-(4-((4′-chloro-4,4-dimethyl-3,4,5,6-tetrahydro-[1,1′-biphenyl]-2-yl)methyl)piperazin-1-yl)-N-((4-(((2R)-4-(5-((2-(2,4-dioxotetrahydropyrimidin-1(2H)-yl)-1-oxoisoindolin-5-yl)methyl)-2,5-diazabicyclo[2.2.2]octan-2-yl)-1-(phenylthio) butan-2-yl)amino)-3-((trifluoromethyl) sulfonyl)phenyl) sulfonyl)benzamide (GT-07811)

With reference to the method of Step 2 in Scheme T-1, the target compound (GT-07811) was obtained (yellow solid, 20 mg, yield 45.44%). ¹H NMR (400 MHz, DMSO-d₆) δ 12.17 (s, 1H), 10.68 (s, 1H), 10.40 (s, 1H), 8.18 (s, 1H), 7.99 (s, 1H), 7.77 (d, J=8.7 Hz, 3H), 7.41 (d, J=8.3 Hz, 2H), 7.30 (d, J=7.7 Hz, 2H), 7.23 (t, J=7.5 Hz, 2H), 7.15 (dd, J=14.2, 7.6 Hz, 3H), 7.01 (s, 1H), 6.95 (d, J=8.9 Hz, 2H), 4.66 (d, J=78.9 Hz, 2H), 4.25 (s, 1H), 3.88 (d, J=12.2 Hz, 3H), 3.78 (t, J=6.9 Hz, 2H), 3.56 (s, 3H), 3.38 (s, 5H), 3.34-3.21 (m, 6H), 2.83 (dd, J=23.6, 15.9 Hz, 4H), 2.22 (d, J=42.2 Hz, 7H), 1.78 (s, 2H), 1.46 (t, J=6.1 Hz, 2H), 1.00 (s, 6H). LCMS (ESI) calcd, for C₆₂H₇₀ClF₃N₉O₈S₃⁺ [M+H]⁺: 1256.42, found, 1256.2.

### Example 297: Preparation of 4-(4-((4′-chloro-4,4-dimethyl-3,4,5,6-tetrahydro-[1,1′-biphenyl]-2-yl)methyl)piperazin-1-yl)-N-((4-(((2R)-4-(5-((2-(2,4-dioxotetrahydropyrimidin-1(2H)-yl)-1,3-dioxoisoindolin-5-yl)methyl)-2,5-diazabicyclo[2.2.2]octan-2-yl)-1-(phenylthio) butan-2-yl)amino)-3-((trifluoromethyl) sulfonyl)phenyl) sulfonyl)benzamide (GT-07815)

With reference to the method of Step 2 in Scheme T-1, the target compound (GT-07815) was obtained (yellow solid, 25 mg, yield 65.55%). ¹H NMR (400 MHz, DMSO-d₆) δ 12.17 (s, 1H), 10.92 (s, 1H), 10.21 (s, 1H), 8.18 (s, 1H), 8.01 (s, 2H), 7.77 (d, J=8.8 Hz, 2H), 7.41 (d, J=8.4 Hz, 2H), 7.30 (d, J=7.8 Hz, 2H), 7.23 (t, J=7.6 Hz, 2H), 7.19-7.12 (m, 3H), 7.01 (s, 1H), 6.96 (d, J=8.9 Hz, 2H), 4.72 (s, 1H), 4.24 (s, 1H), 3.89 (d, J=12.4 Hz, 2H), 3.82 (t, J=6.7 Hz, 2H), 3.57 (s, 3H), 3.40 (s, 6H), 3.37-3.08 (m, 9H), 2.85 (t, J=6.8 Hz, 2H), 2.78 (d, J=8.3 Hz, 2H), 2.22 (d, J=49.0 Hz, 7H), 1.80 (s, 2H), 1.47 (t, J=5.9 Hz, 2H), 1.00 (s, 6H). LCMS (ESI) calcd, for C₆₂H₆₈ClF₃N₉O₉S₃⁺ [M+H]⁺: 1270.39, found, 1270.2.

### Example 298: Preparation of 4-(4-((4′-chloro-4,4-dimethyl-3,4,5,6-tetrahydro-[1,1′-biphenyl]-2-yl)methyl)piperazin-1-yl)-N-((4-(((R)-4-((1R,4R)-5-((2-(2,4-dioxotetrahydropyrimidin-1(2H)-yl)-1-oxoisoindolin-5-yl)methyl)-2,5-diazabicyclo[2.2.1]heptan-2-yl)-1-(phenylthio) butan-2-yl)amino)-3-((trifluoromethyl) sulfonyl)phenyl) sulfonyl)benzamide (GT-07812)

With reference to the method of Step 2 in Scheme T-1, the target compound (GT-07812) was obtained (yellow solid, 20 mg, yield 52.87%). ¹H NMR (500 MHz, DMSO) δ 12.17 (s, 2H), 10.68 (s, 1H), 10.28 (s, 1H), 8.17 (s, 1H), 7.89 (d, J=81.8 Hz, 3H), 7.77 (d, J=8.9 Hz, 2H), 7.41 (d, J=8.5 Hz, 2H), 7.29 (d, J=7.5 Hz, 2H), 7.22 (s, 2H), 7.16 (t, J=9.0 Hz, 3H), 6.95 (d, J=9.1 Hz, 3H), 4.66 (dd, J=117.9, 16.1 Hz, 3H), 4.25 (s, 3H), 3.87 (s, 2H), 3.78 (t, J=7.1 Hz, 2H), 3.57 (s, 10H), 3.33-3.06 (m, 6H), 2.76 (d, J=5.3 Hz, 4H), 2.22 (d, J=59.1 Hz, 7H), 1.47 (t, J=6.3 Hz, 2H), 1.00 (s, 6H). LCMS (ESI) calcd, for C₆₁H₆₈ClF₃N₉O₈S₃⁺ [M+H]⁺: 1242.40, found, 1242.2.

### Example 299: Preparation of 4-(4-((4′-chloro-4,4-dimethyl-3,4,5,6-tetrahydro-[1,1′-biphenyl]-2-yl)methyl)piperazin-1-yl)-N-((4-(((R)-4-((1R,4R)-5-((2-(2,4-dioxotetrahydropyrimidin-1(2H)-yl)-1,3-dioxoisoindolin-5-yl)methyl)-2,5-diazabicyclo[2.2.1]heptan-2-yl)-1-(phenylthio) butan-2-yl)amino)-3-((trifluoromethyl) sulfonyl)phenyl) sulfonyl)benzamide (GT-07816)

With reference to the method of Step 2 in Scheme T-1, the target compound (GT-07816) was obtained (yellow solid, 20 mg, yield 52.27%). ¹H NMR (400 MHz, DMSO-d₆) δ 12.17 (s, 1H), 10.92 (s, 1H), 10.40 (s, 1H), 8.44-7.91 (m, 5H), 7.77 (d, J=8.8 Hz, 2H), 7.41 (d, J=8.3 Hz, 2H), 7.26 (d, J=21.7 Hz, 5H), 7.17 (d, J=8.3 Hz, 3H), 6.96 (d, J=9.0 Hz, 3H), 4.28 (s, 5H), 3.98-3.76 (m, 5H), 3.57 (s, 7H), 3.35-3.05 (m, 7H), 2.86 (t, J=6.8 Hz, 2H), 2.75 (s, 2H), 2.23 (d, J=40.9 Hz, 7H), 1.47 (t, J=6.1 Hz, 2H), 1.00 (s, 6H). LCMS (ESI) calcd, for C₆₁H₆₆ClF₃N₉O₉S₃⁺ [M+H]⁺: 1256.38, found, 1256.3.

### Example 300: Preparation of 4-(4-((4′-chloro-4,4-dimethyl-3,4,5,6-tetrahydro-[1,1′-biphenyl]-2-yl)methyl)piperazin-1-yl)-N-((4-(((2R)-4-(8-((2-(2,4-dioxotetrahydropyrimidin-1(2H)-yl)-1-oxoisoindolin-5-yl)methyl)-3,8-diazabicyclo[3.2.1]octan-3-yl)-1-(phenylthio) butan-2-yl)amino)-3-((trifluoromethyl) sulfonyl)phenyl) sulfonyl)benzamide (GT-07813)

With reference to the method of Step 2 in Scheme T-1, the target compound (GT-07813) was obtained (yellow solid, 12 mg, yield 31.81%). ¹H NMR (400 MHz, DMSO-d₆) δ 12.08 (s, 1H), 10.62 (s, 2H), 10.17 (s, 1H), 8.09 (s, 1H), 7.90 (d, J=7.7 Hz, 2H), 7.79 (s, 2H), 7.70 (d, J=8.8 Hz, 2H), 7.34 (d, J=8.3 Hz, 2H), 7.23 (d, J=7.7 Hz, 2H), 7.16 (t, J=7.6 Hz, 2H), 7.09 (dd, J=12.8, 7.7 Hz, 3H), 7.00 (s, 1H), 6.88 (d, J=8.8 Hz, 3H), 4.61 (dd, J=93.9, 16.2 Hz, 2H), 4.23 (s, 2H), 4.02 (s, 1H), 3.82 (d, J=13.1 Hz, 3H), 3.72 (t, J=6.8 Hz, 2H), 3.51 (s, 2H), 3.35 (s, 2H), 3.31-3.23 (m, 10H), 2.81-2.63 (m, 5H), 2.15 (d, J=48.0 Hz, 7H), 1.69 (s, 3H), 1.41 (d, J=5.6 Hz, 2H), 0.93 (s, 6H). LCMS (ESI) calcd, for C₆₂H₇₀ClF₃N₉O₈S₃⁺ [M+H]⁺: 1256.42, found, 1256.2.

### Example 301: Preparation of 4-(4-((4′-chloro-4,4-dimethyl-3,4,5,6-tetrahydro-[1,1′-biphenyl]-2-yl)methyl)piperazin-1-yl)-N-((4-(((2R)-4-(8-((2-(2,4-dioxotetrahydropyrimidin-1(2H)-yl)-1,3-dioxoisoindolin-5-yl)methyl)-3,8-diazabicyclo[3.2.1]octan-3-yl)-1-(phenylthio) butan-2-yl)amino)-3-((trifluoromethyl) sulfonyl)phenyl) sulfonyl)benzamide (GT-07817)

With reference to the method of Step 2 in Scheme T-1, the target compound (GT-07817) was obtained (yellow solid, 38 mg, yield 74.73%). ¹H NMR (400 MHz, DMSO-d₆) δ 12.16 (s, 1H), 10.93 (s, 1H), 10.48 (s, 1H), 8.37 (d, J=42.0 Hz, 1H), 8.17 (d, J=1.8 Hz, 1H), 8.08 (s, 1H), 8.01-7.92 (m, 1H), 7.77 (d, J=8.9 Hz, 2H), 7.41 (d, J=8.4 Hz, 2H), 7.30 (d, J=7.4 Hz, 2H), 7.24 (d, J=7.4 Hz, 2H), 7.16 (dd, J=14.9, 7.8 Hz, 4H), 6.96 (d, J=8.9 Hz, 3H), 4.24 (d, J=109.4 Hz, 3H), 3.98-3.71 (m, 6H), 3.57 (s, 6H), 3.33 (d, J=29.5 Hz, 8H), 2.92-2.70 (m, 5H), 2.23 (d, J=36.1 Hz, 9H), 1.47 (t, J=6.1 Hz, 2H), 1.00 (s, 6H). LCMS (ESI) calcd. for C₆₂H₆₈ClF₃N₉O₉S₃⁺ [M+H]⁺: 1270.39, found, 1270.2.

### Example 302: Preparation of 4-(4-((4′-chloro-4,4-dimethyl-3,4,5,6-tetrahydro-[1,1′-biphenyl]-2-yl)methyl)piperazin-1-yl)-N-((4-(((2R)-4-(3-((2-(2,4-dioxotetrahydropyrimidin-1(2H)-yl)-1-oxoisoindolin-5-yl)methyl)-3,8-diazabicyclo[3.2.1]octan-8-yl)-1-(phenylthio) butan-2-yl)amino)-3-((trifluoromethyl) sulfonyl)phenyl) sulfonyl)benzamide (GT-07814)

With reference to the method of Step 2 in Scheme T-1, the target compound (GT-07814) was obtained (yellow solid, 14 mg, yield 37.12%). ¹H NMR (400 MHz, DMSO-d₆) δ 12.11 (s, 1H), 10.60 (s, 1H), 10.24 (s, 2H), 8.10 (d, J=2.1 Hz, 1H), 7.93 (d, J=9.2 Hz, 1H), 7.69 (t, J=7.7 Hz, 3H), 7.50 (d, J=31.3 Hz, 2H), 7.34 (d, J=8.4 Hz, 2H), 7.22 (d, J=7.4 Hz, 2H), 7.16 (t, J=7.4 Hz, 3H), 7.10 (t, J=7.6 Hz, 3H), 6.90 (t, J=8.1 Hz, 3H), 4.48 (s, 2H), 4.10 (s, 1H), 3.82 (d, J=13.5 Hz, 4H), 3.70 (d, J=6.0 Hz, 4H), 3.50 (s, 2H), 3.29 (d, J=8.2 Hz, 6H), 2.96 (s, 1H), 2.89-2.62 (m, 9H), 2.09 (t, J=51.3 Hz, 11H), 1.40 (t, J=6.1 Hz, 2H), 0.93 (s, 6H). LCMS (ESI) calcd, for C₆₂H₇₀ClF₃N₉O₈S₃⁺ [M+H]⁺: 1256.42, found, 1256.4.

### Example 303: Preparation of 4-(4-((4′-chloro-4,4-dimethyl-3,4,5,6-tetrahydro-[1,1′-biphenyl]-2-yl)methyl)piperazin-1-yl)-N-((4-(((2R)-4-(3-((2-(2,4-dioxotetrahydropyrimidin-1(2H)-yl)-1,3-dioxoisoindolin-5-yl)methyl)-3,8-diazabicyclo[3.2.1]octan-8-yl)-1-(phenylthio) butan-2-yl)amino)-3-((trifluoromethyl) sulfonyl)phenyl) sulfonyl)benzamide (GT-07821)

With reference to the method of Step 2 in Scheme T-1, the target compound was obtained (GT-07821) (yellow solid, 29 mg, yield 57.08%). ¹H NMR (500 MHz, DMSO) δ 12.11 (s, 1H), 10.84 (s, 1H), 10.42 (d, J=25.8 Hz, 2H), 8.11 (d, J=2.1 Hz, 1H), 7.89 (dd, J=28.4, 7.2 Hz, 4H), 7.70 (d, J=8.9 Hz, 2H), 7.34 (d, J=8.4 Hz, 2H), 7.22 (d, J=7.4 Hz, 2H), 7.16 (dd, J=13.7, 6.3 Hz, 3H), 7.13-7.05 (m, 3H), 6.90 (t, J=9.5 Hz, 3H), 4.12 (d, J=5.0 Hz, 1H), 3.92-3.73 (m, 7H), 3.50 (s, 2H), 3.33-3.24 (m, 8H), 2.96 (s, 1H), 2.83 (d, J=12.4 Hz, 2H), 2.78 (t, J=6.8 Hz, 2H), 2.75-2.60 (m, 4H), 2.16 (d, J=43.8 Hz, 6H), 1.96 (s, 4H), 1.40 (t, J=6.3 Hz, 2H), 0.93 (s, 6H). LCMS (ESI) calcd, for C₆₃H₆₉C₁F₃N₈O₉S₃⁺ [M+H]⁺: 1269.40, found, 1269.2.

### Example 304: Preparation of 1-(5-((4-chloro-2-(7-chloro-5-(trifluoromethyl)-1H-benzo[d]imidazol-2-yl)phenoxy)methyl)-1-oxoisoindolin-2-yl)dihydropyrimidine-2,4 (1H,3H)-dione (GT-06477)

With reference to the method of Step 2 in Scheme T-1, the target compound (GT-06477) was obtained (yellow solid, 21 mg, yield 30.43%). ¹H NMR (400 MHz, DMSO-d₆) δ 10.65 (s, 1H), 8.26 (t, J=7.1 Hz, 1H), 7.98 (s, 1H), 7.83-7.73 (m, 2H), 7.72-7.63 (m, 2H), 7.56-7.46 (m, 1H), 7.25 (d, J=9.0 Hz, 1H), 5.66 (s, 2H), 4.79-4.48 (m, 2H), 3.75 (dd, J=11.3, 6.4 Hz, 2H), 2.87-2.72 (m, 2H). LCMS (ESI) calcd. for C₂₇H₁₉C₁₂F₃N₅O₄⁺ [M+H]⁺: 604.08, found, 604.1.

### Example 305: Preparation of 5-((4-chloro-2-(7-chloro-5-(trifluoromethyl)-1H-benzo[d]imidazol-2-yl)phenoxy)methyl)-2-(2,4-dioxotetrahydropyrimidin-1(2H)-yl)isoindoline-1,3-dione (GT-06478)

With reference to the method of Step 2 in Scheme T-1, the target compound (GT-06478) was obtained (yellow solid, 21 mg, yield 30.43%). ¹H NMR (400 MHz, DMSO-d₆) δ 10.89 (s, 1H), 8.25 (d, J=2.7 Hz, 1H), 8.15 (s, 1H), 8.00 (t, J=14.0 Hz, 3H), 7.68 (s, 1H), 7.54 (d, J=2.7 Hz, 1H), 7.30 (d, J=9.0 Hz, 1H), 5.72 (s, 2H), 3.78 (d, J=6.8 Hz, 2H), 2.83 (t, J=6.8 Hz, 2H). LCMS (ESI) calcd, for C₂₇H₁₇C₁₂F₃N₅O₅⁺ [M+H]⁺: 618.06, found, 618.1.

### Example 306: Preparation of 1-(4-((5-(3,5-dimethylisoxazol-4-yl)-2-methylphenyl)((1-((2-(2,4-dioxotetrahydropyrimidin-1(2H)-yl)-1-oxoisoindolin-5-yl)methyl)piperidin-4-yl)methyl)amino)phenyl)cyclopropane-1-carbonitrile (GT-06409)

With reference to the method of Step 2 in Scheme T-1, the target compound (GT-06409) was obtained (yellow solid, 21 mg, yield 33.14%). ¹H NMR (400 MHz, DMSO-d₆) δ 10.68 (s, 1H), 10.60 (s, 1H), 7.91-7.81 (m, 2H), 7.73 (d, J=7.8 Hz, 1H), 7.43 (d, J=7.9 Hz, 1H), 7.33-7.19 (m, 2H), 7.11 (d, J=8.7 Hz, 2H), 6.51 (dd, J=32.1, 8.6 Hz, 2H), 4.67 (dd, J=91.0, 16.5 Hz, 2H), 4.36 (s, 1H), 3.78 (t, J=6.8 Hz, 2H), 3.51 (d, J=4.6 Hz, 4H), 3.29 (s, 4H), 2.92-2.74 (m, 3H), 2.40 (s, 3H), 2.21 (d, J=14.3 Hz, 3H), 2.04-1.91 (m, 5H), 1.59 (dd, J=7.3, 4.7 Hz, 2H), 1.30 (dd, J=7.6, 4.9 Hz, 2H). LCMS (ESI) calcd, for C₄₁H₄₄N₇O₄⁺ [M+H]⁺: 698.35, found, 698.4.

### Example 307: Preparation of 1-(4-((5-(3,5-dimethylisoxazol-4-yl)-2-methylphenyl)((1-((2-(2,4-dioxotetrahydropyrimidin-1(2H)-yl)-1,3-dioxoisoindolin-5-yl)methyl)piperidin-4-yl)methyl)amino)phenyl)cyclopropane-1-carbonitrile (GT-06410)

With reference to the method of Step 2 in Scheme T-1, the target compound was obtained (GT-06410) (yellow solid, 22 mg, yield 34.05%). ¹H NMR (400 MHz, DMSO-d₆) δ 10.92 (s, 2H), 8.25 (s, 1H), 8.07 (s, 2H), 7.43 (d, J=7.8 Hz, 1H), 7.32-7.18 (m, 2H), 7.11 (d, J=8.6 Hz, 2H), 6.47 (d, J=8.1 Hz, 2H), 4.38 (d, J=40.6 Hz, 2H), 3.81 (t, J=6.7 Hz, 2H), 3.50 (s, 6H), 3.33-3.28 (m, 2H), 2.85 (t, J=6.7 Hz, 3H), 2.40 (s, 3H), 2.23 (s, 3H), 1.97 (d, J=29.7 Hz, 6H), 1.59 (dd, J=7.2, 4.7 Hz, 3H), 1.30 (q, J=5.0 Hz, 2H). LCMS (ESI) calcd. for C₄₁H₄₂N₇O₅⁺ [M+H]⁺: 712.32, found, 712.4.

### Example 308: Preparation of 1-(4-((5-(3,5-dimethylisoxazol-4-yl)-2-methylphenyl)((1-((1-(2-(2,4-dioxotetrahydropyrimidin-1(2H)-yl)-1,3-dioxoisoindolin-5-yl)piperidin-4-yl)methyl)piperidin-4-yl)methyl)amino)phenyl)cyclopropane-1-carbonitrile (GT-06418)

With reference to the method of Scheme T-3, the target compound (GT-06418) was obtained (yellow solid, 11 mg, yield 12.19%). ¹H NMR (400 MHz, DMSO-d₆) δ 10.84 (s, 1H), 9.34 (s, 1H), 7.72 (d, J=8.6 Hz, 1H), 7.45 (d, J=8.0 Hz, 1H), 7.39 (s, 1H), 7.27 (dd, J=17.3, 9.6 Hz, 3H), 7.13 (d, J=8.7 Hz, 2H), 6.51 (t, J=11.1 Hz, 2H), 4.10 (d, J=11.8 Hz, 2H), 3.77 (t, J=6.8 Hz, 2H), 3.52 (s, 5H), 3.01 (d, J=13.3 Hz, 4H), 2.91 (s, 2H), 2.86-2.79 (m, 4H), 2.42 (s, 3H), 2.24 (s, 3H), 2.02 (s, 3H), 1.96 (d, J=10.4 Hz, 2H), 1.84 (d, J=9.7 Hz, 2H), 1.64-1.56 (m, 3H), 1.35-1.28 (m, 2H), 1.23 (s, 2H). LCMS (ESI) calcd, for C₄₆H₅₁N₈O₅⁺ [M+H]⁺: 795.40, found, 795.5.

### Example 309: Preparation of 1-(4-((5-(3,5-dimethylisoxazol-4-yl)-2-methylphenyl)((1-((1-(2-(2,4-dioxotetrahydropyrimidin-1(2H)-yl)-1,3-dioxoisoindolin-4-yl)piperidin-4-yl)methyl)piperidin-4-yl)methyl)amino)phenyl)cyclopropane-1-carbonitrile (GT-06419)

With reference to the method of Scheme T-3, the target compound (GT-06419) was obtained (yellow solid, 10 mg, yield 11.08%). ¹H NMR (400 MHz, DMSO-d₆) δ 10.85 (s, 1H), 9.44 (d, J=80.0 Hz, 1H), 7.80-7.68 (m, 1H), 7.45 (d, J=7.8 Hz, 1H), 7.38 (d, J=6.8 Hz, 2H), 7.23 (s, 2H), 7.12 (d, J=8.7 Hz, 2H), 6.51 (s, 2H), 3.77 (s, 3H), 3.60 (d, J=40.2 Hz, 8H), 3.00-2.83 (m, 6H), 2.42 (s, 3H), 2.24 (s, 3H), 2.02 (s, 3H), 1.98 (s, 9H), 1.60 (d, J=2.6 Hz, 2H), 1.31 (d, J=2.5 Hz, 2H). LCMS (ESI) calcd, for C₄₆H₅₁N₈O₅⁺ [M+H]⁺: 795.40, found, 795.5.

### Example 310: Preparation of 1-(5-((2-(6-methyl-7-oxo-6,7-dihydro-1H-pyrrolo[2,3-c]pyridin-4-yl)-4-(methylsulfonyl)phenoxy)methyl)-1-oxoisoindolin-2-yl)dihydropyrimidine-2,4 (1H,3H)-dione (GT-06413)

With reference to the method of Step 2 in Scheme T-1, the target compound (GT-06413) was obtained (yellow solid, 23 mg, yield 42.40%). ¹H NMR (400 MHz, DMSO-d₆) δ 12.05 (s, 1H), 10.66 (s, 1H), 7.96-7.85 (m, 2H), 7.71 (d, J=8.2 Hz, 1H), 7.55-7.39 (m, 4H), 7.30 (t, J=2.7 Hz, 1H), 6.26-6.13 (m, 1H), 5.39 (s, 2H), 4.72-4.44 (m, 2H), 3.76 (t, J=7.2 Hz, 2H), 3.56 (s, 3H), 3.22 (s, 3H), 2.82 (dd, J=24.7, 16.8 Hz, 2H). LCMS (ESI) calcd, for C₂₈H₂₆N₅O₇S⁺ [M+H]⁺: 576.16, found, 576.2.

### Example 311: Preparation of N-((1r,4r)-4-(3-chloro-4-cyanophenoxy)cyclohexyl)-6-(4-((3-(2-(2,4-dioxotetrahydropyrimidin-1(2H)-yl)-1,3-dioxoisoindolin-5-yl)-3,6-diazabicyclo[3.1.1]heptan-6-yl)methyl)piperidin-1-yl)pyridazine-3-carboxamide (GT-05821)

With reference to the method of Scheme T-3, the target compound (GT-05821) was obtained (yellow solid, 27 mg, yield 31.30%). ¹H NMR (400 MHz, DMSO-d₆) δ 10.85 (d, J=6.1 Hz, 1H), 9.41 (s, 1H), 8.57 (s, 1H), 7.84 (ddd, J=17.2, 9.3, 5.4 Hz, 3H), 7.44-7.34 (m, 2H), 7.24 (d, J=2.0 Hz, 1H), 7.19-7.08 (m, 2H), 4.66 (s, 1H), 4.51 (s, 3H), 4.10-3.77 (m, 6H), 3.44 (s, 2H), 3.01 (d, J=14.5 Hz, 3H), 2.83 (t, J=6.8 Hz, 3H), 2.09 (d, J=17.2 Hz, 3H), 1.87 (s, 5H), 1.57 (dd, J=48.8, 11.0 Hz, 5H), 1.30 (s, 2H). LCMS (ESI) calcd. for C₄₁H₄₄ClN₁₀O₆⁺ [M+H]⁺: 807.31, found, 807.3.

### Example 312: Preparation of N-((1r,4r)-4-(3-chloro-4-cyanophenoxy)cyclohexyl)-6-(4-((3-(2-(2,4-dioxotetrahydropyrimidin-1(2H)-yl)-1,3-dioxoisoindolin-5-yl)-3,8-diazabicyclo[3.2.1]octan-8-yl)methyl)piperidin-1-yl)pyridazine-3-carboxamide (GT-05824)

With reference to the method of Scheme T-3, the target compound (GT-05824) was obtained (yellow solid, 32 mg, yield 36.46%). ¹H NMR (400 MHz, DMSO-d₆) δ 10.86 (s, 1H), 10.53 (s, 1H), 8.60 (d, J=8.2 Hz, 1H), 7.90-7.78 (m, 3H), 7.49-7.37 (m, 3H), 7.36-7.27 (m, 1H), 7.14 (dd, J=8.8, 2.4 Hz, 1H), 4.53 (d, J=10.8 Hz, 3H), 4.21 (s, 2H), 4.03 (d, J=12.5 Hz, 1H), 3.87 (d, J=8.0 Hz, 1H), 3.79 (t, J=6.9 Hz, 2H), 3.71 (d, J=12.1 Hz, 2H), 3.43 (s, 2H), 3.36-3.32 (m, 2H), 3.08 (t, J=12.0 Hz, 2H), 2.99 (s, 1H), 2.84 (t, J=7.0 Hz, 2H), 2.37-2.19 (m, 3H), 2.15-2.07 (m, 2H), 2.04-1.87 (m, 6H), 1.71-1.59 (m, 2H), 1.58-1.47 (m, 2H), 1.31 (d, J=10.1 Hz, 2H). LCMS (ESI) calcd, for C₄₂H₄₆ClN₁₀O₆⁺ [M+H]⁺: 821.33, found, 821.4.

### Example 313: Preparation of N-((1r,4r)-4-(3-chloro-4-cyanophenoxy)cyclohexyl)-6-(4-((4-(1-(2-(2,4-dioxotetrahydropyrimidin-1(2H)-yl)-1,3-dioxoisoindolin-5-yl)piperidin-4-yl)piperazin-1-yl)methyl)piperidin-1-yl)pyridazine-3-carboxamide (GT-05829)

With reference to the method of Scheme T-3, the target compound (GT-05829) was obtained (yellow solid, 30 mg, yield 31.96%). ¹H NMR (400 MHz, DMSO-d₆) δ 11.42 (s, 1H), 10.85 (s, 1H), 8.60 (d, J=8.3 Hz, 1H), 7.85 (dd, J=9.2, 5.7 Hz, 2H), 7.76 (d, J=8.6 Hz, 1H), 7.54-7.33 (m, 4H), 7.13 (dd, J=8.8, 2.4 Hz, 1H), 4.60-4.43 (m, 3H), 4.30 (d, J=13.3 Hz, 2H), 3.96-3.56 (m, 18H), 3.19-2.98 (m, 7H), 2.82 (dd, J=18.8, 12.1 Hz, 2H), 2.29-1.96 (m, 8H), 1.90 (d, J=10.0 Hz, 2H), 1.62 (ddd, J=38.2, 33.7, 23.3 Hz, 7H), 1.35-1.16 (m, 3H). LCMS (ESI) calcd, for C₄₅H₅₃ClN₁₁O₆⁺ [M+H]⁺: 878.39, found, 878.4.

### Example 314: Preparation of N-((1r,4r)-4-(3-chloro-4-cyanophenoxy)cyclohexyl)-6-(4-((5-(2-(2,4-dioxotetrahydropyrimidin-1(2H)-yl)-1,3-dioxoisoindolin-5-yl)-2,5-diazabicyclo[2.2.2]octan-2-yl)methyl)piperidin-1-yl)pyridazine-3-carboxamide (GT-05832)

With reference to the method of Scheme T-3, the target compound (GT-05832) was obtained (yellow solid, 36 mg, yield 41.02%). ¹H NMR (400 MHz, DMSO-d₆) δ 10.84 (s, 1H), 10.30 (s, 1H), 8.58 (d, J=7.6 Hz, 1H), 7.91-7.74 (m, 3H), 7.39 (d, J=11.5 Hz, 2H), 7.26 (s, 1H), 7.17-7.02 (m, 2H), 4.62-4.41 (m, 4H), 3.97 (s, 2H), 3.80 (dd, J=19.1, 11.8 Hz, 4H), 3.59 (dd, J=30.9, 13.9 Hz, 2H), 3.22 (s, 4H), 3.04 (s, 2H), 2.83 (t, J=6.4 Hz, 2H), 2.24-1.83 (m, 10H), 1.72-1.45 (m, 5H), 1.27 (dd, J=23.8, 8.8 Hz, 2H). LCMS (ESI) calcd. for C₄₂H₄₆ClN₁₀O₆⁺ [M+H]⁺: 821.33, found, 821.4.

### Example 315: Preparation of N-((1r,3r)-3-(3-chloro-4-cyanophenoxy)-2,2,4,4-tetramethylcyclobutyl)-6-(4-((3-(2-(2,4-dioxotetrahydropyrimidin-1(2H)-yl)-1,3-dioxoisoindolin-5-yl)-3,6-diazabicyclo[3.1.1]heptan-6-yl)methyl)piperidin-1-yl)nicotinamide (GT-05823)

With reference to the method of Scheme T-3, the target compound (GT-05823) was obtained (yellow solid, 15 mg, yield 22.25%). ¹H NMR (400 MHz, DMSO-d₆) δ 10.96 (s, 1H), 10.86 (d, J=5.7 Hz, 1H), 8.67-8.53 (m, 1H), 8.05 (s, 1H), 7.94-7.82 (m, 2H), 7.75 (d, J=9.0 Hz, 1H), 7.19 (d, J=14.2 Hz, 2H), 7.01 (d, J=8.8 Hz, 2H), 4.66 (s, 1H), 4.55-4.40 (m, 2H), 4.32 (d, J=3.0 Hz, 1H), 4.04 (dd, J=17.2, 12.2 Hz, 4H), 3.88-3.75 (m, 2H), 3.49 (s, 3H), 3.06-2.93 (m, 3H), 2.83 (dd, J=17.9, 12.8 Hz, 3H), 2.11 (s, 1H), 1.97 (d, J=9.9 Hz, 1H), 1.84 (s, 2H), 1.22 (d, J=4.6 Hz, 8H), 1.12 (d, J=2.9 Hz, 6H). LCMS (ESI) calcd, for C₄₄H₄₉ClN₉O₆⁺ [M+H]⁺: 834.35, found, 834.4.

### Example 316: Preparation of N-((1r,3r)-3-(3-chloro-4-cyanophenoxy)-2,2,4,4-tetramethylcyclobutyl)-6-(4-((3-(2-(2,4-dioxotetrahydropyrimidin-1(2H)-yl)-1,3-dioxoisoindolin-5-yl)-3,8-diazabicyclo[3.2.1]octan-8-yl)methyl)piperidin-1-yl)nicotinamide (GT-05826)

With reference to the method of Scheme T-3, the target compound (GT-05826) was obtained (yellow solid, 40 mg, yield 46.68%). ¹H NMR (400 MHz, DMSO-d₆) δ 10.86 (s, 1H), 10.56 (d, J=93.1 Hz, 1H), 8.60 (d, J=2.2 Hz, 1H), 8.10 (d, J=9.0 Hz, 1H), 7.91 (d, J=8.7 Hz, 1H), 7.82 (d, J=8.6 Hz, 2H), 7.46 (s, 1H), 7.31 (d, J=6.7 Hz, 1H), 7.21 (d, J=2.4 Hz, 1H), 7.10 (d, J=9.1 Hz, 1H), 7.01 (dd, J=8.8, 2.4 Hz, 1H), 4.47 (d, J=12.7 Hz, 2H), 4.34 (s, 1H), 4.21 (s, 2H), 4.04 (d, J=18.1 Hz, 2H), 3.78 (dd, J=14.3, 7.4 Hz, 3H), 3.54 (s, 2H), 3.09 (d, J=12.2 Hz, 2H), 2.98 (s, 2H), 2.84 (t, J=6.9 Hz, 2H), 2.21 (s, 3H), 1.99 (dd, J=15.5, 11.1 Hz, 4H), 1.30 (d, J=12.1 Hz, 2H), 1.23 (s, 6H), 1.13 (s, 6H). LCMS (ESI) calcd, for C₄₅H₅₁ClN₉O₆⁺ [M+H]⁺: 848.37, found, 848.4.

### Example 317: Preparation of N-((1r,3r)-3-(3-chloro-4-cyanophenoxy)-2,2,4,4-tetramethylcyclobutyl)-6-(4-((4-(2-(2,4-dioxotetrahydropyrimidin-1(2H)-yl)-4-fluoro-1,3-dioxoisoindolin-5-yl)piperazin-1-yl)methyl)piperidin-1-yl)nicotinamide (GT-05828)

With reference to the method of Scheme T-3, the target compound (GT-05828) was obtained (yellow solid, 32 mg, yield 37.70%). ¹H NMR (400 MHz, DMSO-d₆) δ 10.91 (s, 1H), 10.55 (d, J=38.9 Hz, 1H), 8.60 (d, J=2.1 Hz, 1H), 8.09 (d, J=8.9 Hz, 1H), 7.91 (d, J=8.7 Hz, 1H), 7.79 (t, J=9.4 Hz, 2H), 7.55 (t, J=7.8 Hz, 1H), 7.22 (d, J=2.4 Hz, 1H), 7.09 (d, J=8.8 Hz, 1H), 7.02 (dd, J=8.8, 2.4 Hz, 1H), 4.45 (d, J=13.2 Hz, 2H), 4.33 (s, 1H), 4.07 (d, J=9.1 Hz, 1H), 3.80 (t, J=6.8 Hz, 3H), 3.65 (d, J=10.4 Hz, 2H), 3.53 (s, 2H), 3.27 (s, 3H), 3.14-3.03 (m, 4H), 2.84 (t, J=6.8 Hz, 2H), 2.24 (s, 1H), 1.97 (d, J=11.1 Hz, 2H), 1.26 (d, J=20.0 Hz, 8H), 1.13 (s, 6H). LCMS (ESI) calcd, for C₄₃H₄₈ClFN₇O₆⁺ [M+H]⁺: 840.34, found, 840.4.

### Example 318: Preparation of N-((1r,3r)-3-(3-chloro-4-cyanophenoxy)-2,2,4,4-tetramethylcyclobutyl)-6-(4-((4-(1-(2-(2,4-dioxotetrahydropyrimidin-1(2H)-yl)-1,3-dioxoisoindolin-5-yl)piperidin-4-yl)piperazin-1-yl)methyl)piperidin-1-yl)nicotinamide (GT-05830)

With reference to the method of Scheme T-3, the target compound (GT-05830) was obtained (yellow solid, 32 mg, yield 34.99%). ¹H NMR (400 MHz, DMSO-d₆) δ 12.10-11.09 (m, 1H), 10.84 (s, 1H), 8.58 (s, 1H), 8.07 (d, J=8.8 Hz, 1H), 7.90 (d, J=8.8 Hz, 1H), 7.83-7.71 (m, 2H), 7.48 (s, 1H), 7.36 (d, J=8.4 Hz, 1H), 7.21 (s, 1H), 7.07 (d, J=8.5 Hz, 1H), 7.00 (d, J=8.9 Hz, 1H), 4.42 (d, J=13.3 Hz, 2H), 4.30 (d, J=17.2 Hz, 3H), 4.06 (d, J=9.2 Hz, 1H), 3.79 (d, J=6.6 Hz, 2H), 3.69 (s, 3H), 3.49 (s, 4H), 3.28-3.19 (m, 2H), 3.02 (d, J=11.1 Hz, 6H), 2.83 (t, J=6.5 Hz, 2H), 2.18 (s, 3H), 1.98 (d, J=11.4 Hz, 2H), 1.74 (d, J=10.9 Hz, 2H), 1.22 (s, 8H), 1.12 (s, 6H). LCMS (ESI) calcd, for C₄₈H₅₈ClN₁₀O₆⁺ [M+H]⁺: 905.42, found, 905.6.

### Example 319: Preparation of N-((1r,3r)-3-(3-chloro-4-cyanophenoxy)-2,2,4,4-tetramethylcyclobutyl)-6-(4-((5-(2-(2,4-dioxotetrahydropyrimidin-1(2H)-yl)-1,3-dioxoisoindolin-5-yl)-2,5-diazabicyclo[2.2.2]octan-2-yl)methyl)piperidin-1-yl)nicotinamide (GT-05834)

With reference to the method of Scheme T-3, the target compound (GT-05834) was obtained (yellow solid, 32 mg, yield 37.34%). ¹H NMR (400 MHz, DMSO-d₆) δ 10.85 (s, 2H), 8.58 (s, 1H), 8.15 (d, J=8.3 Hz, 1H), 7.91 (d, J=8.7 Hz, 2H), 7.83-7.74 (m, 1H), 7.21 (d, J=2.3 Hz, 3H), 7.01 (dd, J=8.8, 2.3 Hz, 2H), 4.60 (s, 1H), 4.43 (s, 2H), 4.34 (s, 1H), 4.07 (d, J=9.1 Hz, 3H), 3.79 (t, J=6.8 Hz, 3H), 3.64 (s, 2H), 3.24 (s, 3H), 3.10 (s, 2H), 2.84 (t, J=7.0 Hz, 2H), 2.22 (s, 1H), 1.98 (d, J=13.2 Hz, 5H), 1.23 (s, 8H), 1.13 (s, 6H). LCMS (ESI) calcd, for C₄₅H₅₁ClN₉O₆⁺ [M+H]⁺: 848.37, found, 848.4.

### Example 320: Preparation of 8-(4-(4-((2-(2,4-dioxotetrahydropyrimidin-1(2H)-yl)-1-oxoisoindolin-5-yl)methyl)piperazin-1-yl)piperidin-1-yl)-9-ethyl-6,6-dimethyl-11-oxo-6,11-dihydro-5H-benzo[b]carbazole-3-carbonitrile (GT-06822)

With reference to the method of Scheme T-3, the target compound (GT-06822) was obtained (white solid, 27 mg, yield 46%). ¹H NMR (400 MHz, DMSO-d₆) δ 12.89 (s, 1H), 11.72 (s, 1H), 10.69 (s, 1H), 8.32 (d, J=8.2 Hz, 1H), 8.06 (s, 1H), 8.01 (s, 1H), 7.84 (d, J=13.8 Hz, 2H), 7.78 (s, 1H), 7.61 (d, J=8.2, 1.3 Hz, 1H), 7.36 (s, 1H), 4.81 (d, J=16.5 Hz, 1H), 4.57 (d, J=16.7 Hz, 1H), 4.35 (s, 1H), 3.80 (t, J=6.9 Hz, 2H), 3.70 (s, 2H), 3.47 (s, 6H), 3.32 (d, J=11.1 Hz, 4H), 2.97-2.75 (m, 4H), 2.75-2.67 (m, 2H), 2.22 (d, J=8.9 Hz, 2H), 1.97-1.84 (m, 2H), 1.80 (s, 6H), 1.29 (t, J=7.5 Hz, 3H). LCMS (ESI) calcd, for C₄₃H₄₇N₈O₄⁺ [M+H]⁺: 739.37, found, 739.4.

### Example 321: Preparation of 8-(4-(4-((1-(2-(2,4-dioxotetrahydropyrimidin-1(2H)-yl)-1,3-dioxoisoin dolin-5-yl)piperidin-4-yl)methyl)piperazin-1-yl)piperidin-1-yl)-9-ethyl-6,6-dimethyl-11-oxo-6,11-dihydro-5H-benzo[b]carbazole-3-carbonitrile (GT-06823)

With reference to the method of Scheme T-3, the target compound (GT-06823) was obtained (GT-06823) (white solid, 40 mg, yield 61%). ¹H NMR (400 MHz, DMSO-d₆) δ 12.92 (s, 1H), 10.85 (s, 1H), 8.32 (d, J=8.2 Hz, 1H), 8.07 (s, 1H), 8.01 (s, 1H), 7.73 (d, J=8.6 Hz, 1H), 7.61 (d, J=8.2, 1.1 Hz, 1H), 7.42 (s, 1H), 7.38 (s, 1H), 7.33 (d, J=8.8 Hz, 1H), 4.13 (d, J=12.5 Hz, 2H), 3.79 (t, J=6.8 Hz, 8H), 3.52 (s, 2H), 3.34 (d, J=10.2 Hz, 3H), 3.14-2.98 (m, 4H), 2.88-2.79 (m, 4H), 2.76-2.69 (m, 2H), 2.29-2.15 (m, 3H), 2.06-1.93 (m, 4H), 1.78 (s, 6H), 1.30 (t, J=7.5 Hz, 5H). LCMS (ESI) calcd, for C₄₈H₅₄N₉O₅⁺ [M+H]⁺: 836.42, found, 836.4.

### Example 322: Preparation of 8-(4-(4-((1-(2-(2,4-dioxotetrahydropyrimidin-1(2H)-yl)-1,3-dioxoisoindolin-4-yl)piperidin-4-yl)methyl)piperazin-1-yl)piperidin-1-yl)-9-ethyl-6,6-dimethyl-11-oxo-6,11-dihydro-5H-benzo[b]carbazole-3-carbonitrile (GT-07298)

With reference to the method of Scheme T-3, the target compound (GT-07298) was obtained (white solid, 35 mg, yield 54%). ¹H NMR (400 MHz, DMSO-d₆) δ 12.87 (s, 1H), 12.11 (s, 1H), 11.46 (s, 1H), 10.86 (s, 1H), 8.32 (d, J=8.2 Hz, 1H), 8.07 (s, 1H), 8.01 (s, 1H), 7.78-7.72 (m, 1H), 7.61 (d, J=8.2, 1.3 Hz, 1H), 7.40 (d, J=15.8, 8.6 Hz, 3H), 3.93-3.74 (m, 6H), 3.70 (d, J=11.5 Hz, 2H), 3.57-3.42 (m, 3H), 3.34-3.28 (m, 4H), 3.25-3.06 (m, 2H), 2.99-2.90 (m, 2H), 2.89-2.81 (m, 4H), 2.77-2.70 (m, 2H), 2.24 (s, 2H), 2.13-1.87 (m, 5H), 1.77 (s, 6H), 1.42 (s, 2H), 1.30 (t, J=7.5 Hz, 3H). LCMS (ESI) calcd, for C₄₈H₅₄N₉O₅⁺ [M+H]⁺: 836.42, found, 836.5.

### Example 323: Preparation of 8-(4-((2-(2,4-dioxotetrahydropyrimidin-1(2H)-yl)-1,3-dioxoisoindolin-5-yl)methyl)piperazin-1-yl)-9-ethyl-6,6-dimethyl-11-oxo-6,11-dihydro-5H-benzo[b]carbazole-3-carbonitrile (GT-07320)

With reference to the method of Scheme T-3, the target compound (GT-07320) was obtained (white solid, 14 mg, yield 43%). ¹H NMR (400 MHz, DMSO-d₆) δ 12.87 (s, 1H), 11.33 (s, 1H), 10.94 (s, 1H), 8.37 (s, 1H), 8.31 (d, J=8.2 Hz, 1H), 8.23 (s, 1H), 8.14 (d, J=6.5 Hz, 1H), 8.08 (s, 1H), 8.01 (s, 1H), 7.61 (d, J=8.8 Hz, 1H), 7.38 (s, 1H), 4.68 (s, 1H), 3.84 (t, J=6.7 Hz, 2H), 3.55-3.40 (m, 2H), 3.35 (s, 4H), 3.30-3.14 (m, 3H), 2.87 (t, J=6.8 Hz, 2H), 2.76-2.69 (m, 2H), 1.77 (s, 6H), 1.34-1.24 (m, 3H). LCMS (ESI) calcd. for C₃₈H₃₆N₇O₅⁺ [M+H]⁺: 670.28, found, 670.3.

### Example 324: Preparation of 8-(4-((1-(2-(2,4-dioxotetrahydropyrimidin-1(2H)-yl)-1,3-dioxoisoindolin-5-yl)piperidin-4-yl)methyl)piperazin-1-yl)-9-ethyl-6,6-dimethyl-11-oxo-6,11-dihydro-5H-benzo[b]carbazole-3-carbonitrile (GT-07321)

With reference to the method of Scheme T-3, the target compound (GT-07321) was obtained (white solid, 20 mg, yield 55%). ¹H NMR (400 MHz, DMSO-d₆) δ 12.90 (s, 1H), 10.85 (s, 1H), 10.28 (s, 1H), 8.32 (d, J=8.2 Hz, 1H), 8.10 (s, 1H), 8.02 (s, 1H), 7.74 (d, J=8.6 Hz, 1H), 7.62 (dd, J=8.2, 1.3 Hz, 1H), 7.44 (d, J=1.9 Hz, 1H), 7.39 (s, 1H), 7.34 (dd, J=8.8, 2.1 Hz, 1H), 4.15 (d, J=13.3 Hz, 2H), 3.79 (t, J=6.9 Hz, 2H), 3.63 (d, J=9.4 Hz, 2H), 3.36 (s, 4H), 3.30-3.22 (m, 2H), 3.16 (s, 2H), 3.06 (t, J=12.1 Hz, 2H), 2.84 (t, J=7.2 Hz, 2H), 2.79-2.70 (m, 2H), 2.24 (s, 1H), 1.95 (d, J=11.0 Hz, 2H), 1.79 (s, 6H), 1.39-1.26 (m, 5H). LCMS (ESI) calcd, for C₄₃H₄₅N₈O₅⁺[M+H]⁺: 753.35, found, 753.4.

### Example 325: Preparation of 8-(4-((1-(2-(2,4-dioxotetrahydropyrimidin-1(2H)-yl)-1,3-dioxoisoindolin-4-yl)piperidin-4-yl)methyl)piperazin-1-yl)-9-ethyl-6,6-dimethyl-11-oxo-6,11-dihydro-5H-benzo[b]carbazole-3-carbonitrile (GT-07322)

With reference to the method of Scheme T-3, the target compound (GT-07322) was obtained (white solid, 22 mg, yield 61%). ¹H NMR (400 MHz, DMSO-d₆) δ 12.93 (s, 1H), 10.87 (s, 1H), 10.42 (s, 1H), 8.33 (d, J=8.2 Hz, 1H), 8.10 (s, 1H), 8.02 (s, 1H), 7.76 (t, J=7.8 Hz, 1H), 7.69-7.57 (m, 1H), 7.47-7.32 (m, 3H), 3.86-3.76 (m, 3H), 3.72 (d, J=11.1 Hz, 1H), 3.65 (d, J=9.5 Hz, 2H), 3.38 (s, 2H), 3.34-3.25 (m, 4H), 3.22 (s, 2H), 3.05-2.91 (m, 2H), 2.89-2.80 (m, 2H), 2.80-2.71 (m, 2H), 2.15 (s, 1H), 2.02 (d, J=11.6 Hz, 2H), 1.80 (s, 6H), 1.61-1.42 (m, 2H), 1.30 (t, J=7.5 Hz, 3H). LCMS (ESI) calcd, for C₄₃H₄₅N₈O₅⁺ [M+H]⁺: 753.35, found, 753.3.

### Example 326: Preparation of 8-(4-((2-(2,4-dioxotetrahydropyrimidin-1(2H)-yl)-1-oxoisoindolin-5-yl)methyl)piperazin-1-yl)-9-ethyl-6,6-dimethyl-11-oxo-6,11-dihydro-5H-benzo[b]carbazole-3-carbonitrile (GT-07340)

With reference to the method of Scheme T-3, the target compound (GT-07340) was obtained (white solid, 18 mg, yield 56%). ¹H NMR (400 MHz, DMSO-d₆) δ 12.88 (s, 1H), 11.16 (s, 1H), 10.69 (d, J=11.6 Hz, 1H), 8.31 (d, J=8.2 Hz, 1H), 8.09 (s, 1H), 8.01 (s, 1H), 7.99-7.80 (m, 3H), 7.61 (d, J=8.3 Hz, 1H), 7.37 (s, 1H), 4.92-4.78 (m, 1H), 4.61 (s, 3H), 3.80 (t, J=6.8 Hz, 2H), 3.42 (s, 2H), 3.34-3.21 (m, 6H), 2.92-2.82 (m, 1H), 2.82-2.67 (m, 3H), 1.77 (s, 6H), 1.29 (t, J=7.4 Hz, 3H). LCMS (ESI) calcd, for C₃₈H₃₈N₇O₄⁺ [M+H]⁺: 656.30, found, 656.4.

### Example 327: Preparation of 1-(5-((4-(4-((5-chloro-4-((2-(dimethylphosphoryl)phenyl)amino)pyrimidin-2-yl)amino)-3-methoxyphenyl)piperazin-1-yl)methyl)-1-oxoisoindolin-2-yl)dihydropyrimidine-2,4 (1H,3H)-dione (GT-07341)

With reference to the method of Scheme T-3, the target compound (GT-07341) was obtained (white solid, 25 mg, yield 48%). ¹H NMR (400 MHz, DMSO-d₆) δ 11.93 (s, 1H), 11.75 (s, 1H), 10.70 (s, 1H), 9.33 (s, 1H), 8.37 (s, 1H), 8.22 (d, J=26.8 Hz, 1H), 7.95 (d, J=23.8 Hz, 1H), 7.92-7.77 (m, 2H), 7.67-7.52 (m, 1H), 7.37 (d, J=8.7 Hz, 2H), 7.23 (t, J=7.2 Hz, 1H), 6.74 (d, J=2.1 Hz, 1H), 6.56 (d, J=8.7, 2.1 Hz, 1H), 4.83 (d, J=16.9 Hz, 1H), 4.58 (d, J=24.2 Hz, 3H), 3.88 (d, J=10.4 Hz, 2H), 3.81 (d, J=6.9 Hz, 2H), 3.79 (s, 3H), 3.43 (d, J=9.4 Hz, 2H), 3.31-3.20 (m, 4H), 2.91-2.82 (m, 1H), 2.82-2.71 (m, 1H), 1.79 (d, J=13.6 Hz, 6H). LCMS (ESI) calcd, for C₃₆H₄₀ClN₉O₅P⁺ [M+H]⁺: 744.26, found, 744.3.

### Example 328: Preparation of 5-((4-(4-((5-chloro-4-((2-(dimethylphosphoryl)phenyl)amino)pyrimidin-2-yl)amino)-3-methoxyphenyl)piperazin-1-yl)methyl)-2-(2,4-dioxotetrahydropyrimidin-1(2H)-yl)isoindoline-1,3-dione (GT-07342)

With reference to the method of Scheme T-3, the target compound (GT-07342) was obtained (white solid, 19 mg, yield 35%). ¹H NMR (400 MHz, DMSO-d₆) δ 11.98 (s, 2H), 10.93 (s, 1H), 9.39 (s, 1H), 8.39 (s, 1H), 8.26 (d, J=6.6 Hz, 2H), 8.13 (d, J=7.7 Hz, 1H), 7.68-7.54 (m, 1H), 7.37 (d, J=8.6 Hz, 2H), 7.24 (t, J=7.2 Hz, 1H), 6.74 (d, J=2.1 Hz, 1H), 6.64-6.47 (m, 1H), 4.73-4.53 (m, 2H), 3.88 (s, 2H), 3.83 (t, J=6.7 Hz, 2H), 3.79 (s, 3H), 3.44 (d, J=5.9 Hz, 2H), 3.25 (s, 4H), 2.87 (t, J=6.7 Hz, 2H), 1.80 (d, J=13.6 Hz, 6H). LCMS (ESI) calcd, for C₃₆H₃₈ClN₇O₆P⁺ [M+H]⁺: ,758.24, found, 758.3.

### Example 329: Preparation of 5-(4-((4-(4-((5-chloro-4-((2-(dimethylphosphoryl)phenyl)amino)pyrimidin-2-yl)amino)-3-methoxyphenyl)piperazin-1-yl)methyl)piperidin-1-yl)-2-(2,4-dioxotetrahydropyrimidin-1(2H)-yl)isoindoline-1,3-dione (GT-07343)

With reference to the method of Scheme T-3, the target compound (GT-07343) was obtained (yellow solid, 18 mg, yield 30%). ¹H NMR (400 MHz, DMSO-d₆) δ 12.13 (s, 1H), 10.85 (s, 1H), 10.85 (s, 1H), 9.62 (s, 1H), 8.32 (s, 1H), 8.30 (s, 1H), 7.76-7.70 (m, 1H), 7.65 (dd, J=13.5, 8.0 Hz, 1H), 7.46-7.40 (m, 2H), 7.38-7.31 (m, 2H), 7.27 (t, J=7.5 Hz, 1H), 6.77 (d, J=2.1 Hz, 1H), 6.61 (d, J=8.9 Hz, 1H), 4.13 (d, J=13.3 Hz, 2H), 3.89-3.86 (m, 3H), 3.81 (s, 3H), 3.78 (d, J=6.3 Hz, 3H), 3.62 (d, 2H), 3.38 (t, J=11.5 Hz, 2H), 3.19-3.16 (m, 1H), 3.10-3.03 (m, 3H), 2.82 (t, J=6.8 Hz, 2H), 2.25 (s, 1H), 2.00 (d, J=11.9 Hz, 2H), 1.82 (s, 3H), 1.79 (s, 3H), 1.36-1.22 (m, 2H). LCMS (ESI) calcd, for C₄₁H₄₇ClN₁₀O₆P⁺ [M+H]⁺: 841.31, found, 841.4.

### Example 330: Preparation of 4-(4-((4-(4-((5-chloro-4-((2-(dimethylphosphoryl)phenyl)amino)pyrimidin-2-yl)amino)-3-methoxyphenyl)piperazin-1-yl)methyl)piperidin-1-yl)-2-(2,4-dioxotetrahydropyrimidin-1(2H)-yl)isoindoline-1,3-dione (GT-07344)

With reference to the method of Scheme T-3, the target compound (GT-07344) was obtained (yellow solid, 14 mg, yield 23%). ¹H NMR (400 MHz, DMSO-d₆) δ 12.16 (s, 1H), 10.97 (s, 1H), 10.86 (s, 1H), 9.69 (s, 1H), 8.31 (s, 2H), 7.79-7.73 (m, 1H), 7.65 (dd, J=13.6, 7.7 Hz, 1H), 7.44-7.39 (m, 2H), 7.37 (d, J=8.4 Hz, 1H), 7.28 (t, J=7.2 Hz, 1H), 6.77 (d, J=2.0 Hz, 1H), 6.62 (d, J=6.7 Hz, 1H), 3.87 (d, 3H), 3.81 (s, 3H), 3.77-3.73 (m, 3H), 3.69-3.62 (m, 4H), 3.41 (t, J=12.2 Hz, 2H), 3.20 (s, 1H), 3.18-3.13 (m, 2H), 3.00-2.89 (m, 2H), 2.86-2.80 (m, 2H), 2.13 (s, 1H), 2.10-1.99 (m, 2H), 1.82 (s, 3H), 1.79 (s, 3H), 1.58-1.37 (m, 2H). LCMS (ESI) calcd, for C₄₁H₄₇ClN₁₀O₆P⁺ [M+H]⁺: 841.31, found, 841.4.

### Example 331: Preparation of 1-(5-((4-(4-((5-chloro-4-((2-(isopropylsulfonyl)phenyl)amino)pyrimidin-2-yl)amino)-5-isopropoxy-2-methylphenyl)piperidin-1-yl)methyl)-1-oxoisoindolin-2-yl)dihydropyrimidine-2,4 (1H,3H)-dione (GT-07362)

With reference to the method of Scheme T-3, the target compound (GT-07362) was obtained (white solid, 45 mg, yield 74%). ¹H NMR (400 MHz, DMSO-d₆) δ 11.26 (s, 1H), 10.70 (s, 1H), 9.80 (s, 1H), 8.73 (s, 1H), 8.38 (s, 1H), 8.27 (d, J=8.0 Hz, 1H), 7.96 (s, 1H), 7.93-7.81 (m, 3H), 7.67 (t, J=7.2 Hz, 1H), 7.44 (t, J=7.7 Hz, 1H), 7.39 (s, 1H), 6.85 (s, 1H), 4.83 (d, J=16.7 Hz, 1H), 4.59 (d, J=16.8 Hz, 1H), 4.49-4.43 (m, 2H), 3.80 (t, 3H), 3.48-3.40 (m, 3H), 3.15-3.02 (m, 2H), 2.98-2.82 (m, 2H), 2.81-2.71 (m, 1H), 2.26-2.12 (m, 2H), 2.07 (s, 3H), 1.84 (d, J=13.3 Hz, 2H), 1.25 (d, J=10.3, 4.6 Hz, 6H), 1.14 (d, J=6.8 Hz, 6H). LCMS (ESI) calcd, for C₄₁H₄₈ClN₈O₆S⁺ [M+H]⁺: 815.31, found, 815.4.

### Example 332: Preparation of 5-((4-(4-((5-chloro-4-((2-(isopropylsulfonyl)phenyl)amino)pyrimidin-2-yl)amino)-5-isopropoxy-2-methylphenyl)piperidin-1-yl)methyl)-2-(2,4-dioxotetrahydropyrimidin-1(2H)-yl)isoindoline-1,3-dione (GT-07363)

With reference to the method of Scheme T-3, the target compound (GT-07363) was obtained (white solid, 25 mg, yield 40%). ¹H NMR (400 MHz, DMSO-d₆) δ 11.28 (s, 1H), 10.94 (s, 1H), 9.72 (s, 1H), 8.58 (s, 1H), 8.35 (d, J=6.6 Hz, 2H), 8.31 (d, J=8.4 Hz, 1H), 8.22 (d, J=7.7 Hz, 1H), 8.14 (d, J=8.7 Hz, 1H), 7.87 (d, J=8.0 Hz, 1H), 7.64 (t, J=21.3, 13.6 Hz, 1H), 7.46-7.36 (m, 2H), 6.81 (s, 1H), 4.57 (s, 1H), 4.51-4.44 (m, 1H), 3.83 (t, J=6.7 Hz, 2H), 3.48-3.44 (m, 3H), 3.16-3.06 (m, 2H), 2.93 (t, J=12.0 Hz, 1H), 2.87 (t, J=6.7 Hz, 2H), 2.21-2.11 (m, 2H), 2.09 (s, 2H), 1.86 (d, J=12.6 Hz, 2H), 1.24 (t, J=6.0 Hz, 6H), 1.14 (d, J=6.7 Hz, 6H). LCMS (ESI) calcd, for C₄₁H₄₆ClN₈O₇S⁺ [M+H]⁺: 829.29, found, 829.3.

### Example 333: Preparation of 1-(5-((4-(1-(4-((5-chloro-4-((2-(dimethylphosphoryl)phenyl)amino)pyrimidin-2-yl)amino)-3-methoxyphenyl)piperidin-4-yl)piperazin-1-yl)methyl)-1-oxoisoindolin-2-yl)dihydropyrimidine-2,4 (1H,3H)-dione (GT-08146)

With reference to the method of Scheme T-3, the target compound (GT-08146) was obtained (white solid, 35 mg, yield 59%). ¹H NMR (400 MHz, DMSO-d₆) δ 12.09 (s, 1H), 10.68 (s, 1H), 9.60 (s, 1H), 8.20 (d, J=91.3 Hz, 2H), 7.96 (s, 1H), 7.83 (s, 2H), 7.69-7.61 (m, 1H), 7.45 (s, 2H), 7.28 (t, J=7.2 Hz, 1H), 6.95 (s, 1H), 6.76 (s, 1H), 4.81 (d, J=16.8 Hz, 1H), 4.57 (d, J=15.4 Hz, 2H), 3.91 (d, J=10.9 Hz, 4H), 3.82-3.79 (m, 6H), 3.64-3.60 (m, 3H), 3.55-3.51 (m, 2H), 2.98 (s, 2H), 2.89-2.83 (m, 1H), 2.80-2.74 (m, 1H), 2.26 (d, J=9.0 Hz, 2H), 1.98 (s, 2H), 1.82 (s, 3H), 1.79 (s, 3H). LCMS (ESI) calcd, for C₄₁H₄₉ClN₁₀O₅P⁺ [M+H]⁺: 827.33, found, 827.2.

### Example 334: Preparation of 5-((4-(1-(4-((5-chloro-4-((2-(dimethylphosphoryl)phenyl)amino)pyrimidin-2-yl)amino)-3-methoxyphenyl)piperidin-4-yl)piperazin-1-yl)methyl)-2-(2,4-dioxotetrahydropyrimidin-1(2H)-yl)isoindoline-1,3-dione (GT-08147)

With reference to the method of Scheme T-3, the target compound (GT-08147) was obtained (white solid, 39 mg, yield 64%). ¹H NMR (400 MHz, DMSO-d₆) δ 12.05 (s, 1H), 10.93 (s, 1H), 9.51 (s, 1H), 8.34 (s, 1H), 8.30 (d, J=13.2 Hz, 2H), 8.19 (d, J=7.4 Hz, 1H), 8.09 (d, J=7.7 Hz, 1H), 7.68-7.61 (m, 1H), 7.44 (s, 2H), 7.27 (t, J=7.4 Hz, 1H), 6.91 (s, 1H), 6.73 (s, 1H), 4.51 (s, 2H), 3.96-3.87 (m, 4H), 3.86-3.79 (m, 8H), 3.50-3.46 (m, 4H), 2.93 (s, 2H), 2.86 (t, J=6.7 Hz, 2H), 2.24 (s, 2H), 1.97 (s, 2H), 1.82 (s, 3H), 1.78 (s, 3H). LCMS (ESI) calcd, for C₄₁H₄₇ClN₁₀O₆P⁺ [M+H]⁺: 841.31, found, 841.1.

### Example 335: Preparation of 1-(5-(((1-(4-((5-chloro-4-((2-(dimethylphosphoryl)phenyl)amino)pyrimidin-2-yl)amino)-3-methoxyphenyl)piperidin-4-yl)(methyl)amino)methyl)-1-oxoisoindolin-2-yl)dihydropyrimidine-2,4 (1H,3H)-dione (GT-08148)

With reference to the method of Scheme T-3, the target compound (GT-08148) was obtained (white solid, 42 mg, yield 67%). ¹H NMR (400 MHz, DMSO-d₆) δ 12.04 (s, 1H), 11.42 (s, 1H), 10.68 (s, 1H), 9.54 (s, 1H), 8.32 (s, 2H), 8.01 (s, 1H), 7.93-7.85 (m, 2H), 7.68-7.62 (m, 1H), 7.55-7.39 (m, 2H), 7.27 (t, J=7.1 Hz, 1H), 7.03 (s, 1H), 6.87 (s, 1H), 4.90-4.78 (m, 1H), 4.68-4.56 (m, 2H), 3.90-3.88 (m, 2H), 3.83 (s, 4H), 3.80-3.79 (m, 2H), 3.54-3.51 (m, 1H), 3.08 (s, 2H), 2.92-2.85 (m, 1H), 2.81-2.75 (m, 1H), 2.65 (d, J=4.0 Hz, 3H), 2.42 (d, J=11.2 Hz, 1H), 2.36-2.30 (m, 1H), 2.25-2.07 (m, 2H), 1.82 (s, 3H), 1.79 (s, 3H). LCMS (ESI) calcd, for C₃₈H₄₄ClN₇O₅P⁺ [M+H]⁺: 772.29, found, 772.1.

### Example 336: Preparation of 5-(4-((4-(4-((5-chloro-4-((2-(isopropylsulfonyl)phenyl)amino)pyrimidin-2-yl)amino)-5-isopropoxy-2-methylphenyl)piperidin-1-yl)methyl)piperidin-1-yl)-2-(2,4-dioxotetrahydropyrimidin-1(2H)-yl)isoindoline-1,3-dione (GT-08249)

With reference to the method of Scheme T-3, the target compound (GT-08249) was obtained (yellow solid, 30 mg, yield 44%). ¹H NMR (400 MHz, DMSO-d₆) δ 10.85 (s, 1H), 10.06 (s, 1H), 9.69 (s, 1H), 8.50 (s, 1H), 8.35 (s, 2H), 7.88 (d, J=8.0 Hz, 1H), 7.74 (d, J=8.6 Hz, 1H), 7.67 (t, J=7.5 Hz, 1H), 7.48-7.39 (m, 3H), 7.33 (d, J=8.7 Hz, 1H), 6.88 (s, 1H), 4.56-4.46 (m, 1H), 4.14 (d, J=13.1 Hz, 2H), 3.79 (t, 3H), 3.61 (d, J=11.7 Hz, 2H), 3.49-3.43 (m, 1H), 3.08-2.98 (m, 5H), 2.83 (t, J=6.9 Hz, 2H), 2.26-2.16 (m, 2H), 2.12 (s, 3H), 1.98 (d, J=11.3 Hz, 2H), 1.84 (d, J=12.0 Hz, 2H), 1.35-1.28 (m, 2H), 1.24 (d, J=6.0 Hz, 6H), 1.15 (d, J=6.8 Hz, 6H). LCMS (ESI) calcd, for C₄₆H₅₅ClN₇O₇S⁺ [M+H]⁺: 912.36, found, 912.2.

### Example 337: Preparation of 4-(4-((4-(4-((5-chloro-4-((2-(isopropylsulfonyl)phenyl)amino)pyrimidin-2-yl)amino)-5-isopropoxy-2-methylphenyl)piperidin-1-yl)methyl)piperidin-1-yl)-2-(2,4-dioxotetrahydropyrimidin-1(2H)-yl)isoindoline-1,3-dione (GT-08250)

With reference to the method of Scheme T-3, the target compound (GT-08250) was obtained (yellow solid, 32 mg, yield 47%). ¹H NMR (400 MHz, DMSO-d₆) δ 10.86 (s, 1H), 10.22 (s, 1H), 9.81 (s, 1H), 8.72 (s, 1H), 8.40 (s, 1H), 8.29 (d, J=7.9 Hz, 1H), 7.89 (d, J=7.9 Hz, 1H), 7.79-7.67 (m, 2H), 7.49-7.38 (m, 4H), 6.91 (s, 1H), 4.57-4.47 (m, 1H), 3.84-3.75 (m, 4H), 3.71 (d, J=12.4 Hz, 1H), 3.62 (d, J=10.4 Hz, 2H), 3.50-3.44 (m, 1H), 3.16-3.02 (m, 4H), 3.01-2.94 (m, 2H), 2.87-2.79 (m, 2H), 2.37-2.25 (m, 2H), 2.10 (s, 4H), 2.04 (s, 2H), 1.83 (d, J=12.2 Hz, 2H), 1.56-1.41 (m, 2H), 1.25 (d, J=6.0 Hz, 6H), 1.15 (d, J=6.8 Hz, 6H). LCMS (ESI) calcd, for C₄₆H₅₅ClN₇O₇S⁺ [M+H]⁺: 912.36, found, 912.2.

### Example 338: Preparation of 5-(4-((4-(1-(4-((5-chloro-4-((2-(dimethylphosphoryl)phenyl)amino)pyrimidin-2-yl)amino)-3-methoxyphenyl)piperidin-4-yl)piperazin-1-yl)methyl)piperidin-1-yl)-2-(2,4-dioxotetrahydropyrimidin-1(2H)-yl)isoindoline-1,3-dione (GT-08251)

With reference to the method of Scheme T-3, the target compound (GT-08251) was obtained (yellow solid, 22 mg, yield 34%). ¹H NMR (400 MHz, DMSO-d₆) δ 12.01 (s, 1H), 11.68 (s, 1H), 10.84 (s, 1H), 8.36 (s, 1H), 8.28 (s, 1H), 7.72 (d, J=8.6 Hz, 1H), 7.64 (dd, J=13.9, 7.4 Hz, 1H), 7.48-7.38 (m, 3H), 7.33-7.24 (m, 2H), 6.89 (s, 1H), 6.73 (s, 1H), 4.12 (d, J=12.7 Hz, 2H), 3.95-3.89 (m, 4H), 3.82 (s, 3H), 3.77 (t, J=6.8 Hz, 6H), 3.52 (s, 4H), 3.10 (s, 1H), 3.02 (t, J=12.2 Hz, 3H), 2.96-2.90 (m, 1H), 2.83 (t, J=7.2 Hz, 2H), 2.30-2.17 (m, 3H), 2.05-1.90 (m, 4H), 1.82 (s, 3H), 1.79 (s, 3H), 1.31-1.21 (m, 2H). LCMS (ESI) calcd. for C₄₆H₅₆ClN₁₁O₆P⁺[M+H]⁺: 924.38, found, 924.2.

### Example 339: Preparation of 4-(4-((4-(1-(4-((5-chloro-4-((2-(dimethylphosphoryl)phenyl)amino)pyrimidin-2-yl)amino)-3-methoxyphenyl)piperidin-4-yl)piperazin-1-yl)methyl)piperidin-1-yl)-2-(2,4-dioxotetrahydropyrimidin-1(2H)-yl)isoindoline-1,3-dione (GT-08252)

With reference to the method of Scheme T-3, the target compound (GT-08252) was obtained (yellow solid, 31 mg, yield 48%). ¹H NMR (400 MHz, DMSO-d₆) δ 12.42 (s, 1H), 12.04 (s, 1H), 11.73 (s, 1H), 10.85 (s, 1H), 9.48 (s, 1H), 8.36 (s, 1H), 8.29 (s, 1H), 7.81-7.70 (m, 1H), 7.65 (dd, J=14.1, 7.5 Hz, 1H), 7.47-7.43 (m, 1H), 7.40 (d, J=7.3 Hz, 2H), 7.28 (t, J=7.2 Hz, 1H), 6.92 (s, 1H), 6.75 (s, 1H), 3.95-3.92 (m, 2H), 3.82 (s, 4H), 3.79-3.76 (m, 4H), 3.71-3.66 (m, 6H), 3.57-3.51 (m, 4H), 3.17 (s, 1H), 3.00-2.90 (m, 3H), 2.83 (t, J=7.2 Hz, 2H), 2.32-2.23 (m, 2H), 2.09 (s, 3H), 2.00 (s, 2H), 1.82 (s, 3H), 1.79 (s, 3H), 1.55-1.36 (m, 2H). LCMS (ESI) calcd, for C₄₆H₅₆ClN₁₁O₆P⁺[M+H]⁺: 924.38, found, 924.2.

### Example 340: Preparation of 5-(4-(((1-(4-((5-chloro-4-((2-(dimethylphosphoryl)phenyl)amino)pyrimidin-2-yl)amino)-3-methoxyphenyl)piperidin-4-yl)(methyl)amino)methyl)piperidin-1-yl)-2-(2,4-dioxotetrahydropyrimidin-1(2H)-yl)isoindoline-1,3-dione (GT-08253)

With reference to the method of Scheme T-3, the target compound (GT-08253) was obtained (yellow solid, 50 mg, yield 71%). ¹H NMR (400 MHz, DMSO-d₆) δ 12.05 (s, 1H), 10.84 (s, 1H), 10.48 (s, 1H), 9.55 (s, 1H), 8.47-8.18 (m, 2H), 7.73 (d, J=8.6 Hz, 1H), 7.65 (dd, J=13.6, 7.7 Hz, 1H), 7.49-7.45 (m, 1H), 7.43-7.40 (m, 1H), 7.33 (d, J=8.7 Hz, 1H), 7.26 (t, J=7.3 Hz, 1H), 7.04 (s, 1H), 6.86 (s, 1H), 4.14 (d, J=12.8 Hz, 2H), 3.87-3.85 (m, 3H), 3.82 (s, 3H), 3.78 (t, J=6.0 Hz, 6H), 3.57-3.51 (m, 2H), 3.17-3.10 (m, 2H), 2.94 (s, 1H), 2.83 (t, J=7.2 Hz, 2H), 2.78 (d, J=4.6 Hz, 2H), 2.34-2.29 (m, 1H), 2.22-2.16 (m, 2H), 2.09-2.02 (m, 1H), 1.89 (d, J=11.9 Hz, 1H), 1.82 (s, 3H), 1.78 (s, 3H), 1.36-1.21 (m, 2H). LCMS (ESI) calcd. for C₄₃H₅₁ClN₁₀O₆P⁺[M+H]⁺: 869.34, found, 869.2.

### Example 341: Preparation of 4-(4-(((1-(4-((5-chloro-4-((2-(dimethylphosphoryl)phenyl)amino)pyrimidin-2-yl)amino)-3-methoxyphenyl)piperidin-4-yl)(methyl)amino)methyl)piperidin-1-yl)-2-(2,4-dioxotetrahydropyrimidin-1(2H)-yl)isoindoline-1,3-dione (GT-08254)

With reference to the method of Scheme T-3, the target compound (GT-08254) was obtained (yellow solid, 27 mg, yield 39%). ¹H NMR (400 MHz, DMSO-d₆) δ 12.02 (s, 1H), 10.86 (s, 1H), 10.45 (s, 1H), 9.50 (s, 1H), 8.39 (s, 1H), 8.31 (s, 1H), 7.78-7.72 (m, 1H), 7.65 (dd, J=13.7, 7.4 Hz, 1H), 7.46 (s, 1H), 7.44-7.38 (m, 2H), 7.27 (t, J=7.1 Hz, 1H), 7.00 (s, 1H), 6.83 (s, 1H), 3.91-3.86 (m, 3H), 3.83 (s, 3H), 3.80 (d, J=6.4 Hz, 3H), 3.77 (d, J=4.7 Hz, 2H), 3.23-3.19 (m, 1H), 3.10 (s, 2H), 3.03-2.95 (m, 3H), 2.87-2.82 (m, 2H), 2.81-2.78 (m, 3H), 2.33 (s, 1H), 2.28-2.21 (m, 2H), 2.08 (s, 2H), 1.93 (d, J=11.0 Hz, 1H), 1.82 (s, 3H), 1.78 (s, 3H), 1.53-1.39 (m, 2H). LCMS (ESI) calcd, for C₄₃H₅₁ClN₁₀O₆P⁺[M+H]⁺: 869.34, found, 869.2.

### Example 342: Preparation of N-((4,6-dimethyl-2-oxo-1,2-dihydropyridin-3-yl)methyl)-4′-(4-(1-((1-(2-(2,4-dioxotetrahydropyrimidin-1(2H)-yl)-1,3-dioxoisoindolin-5-yl)piperidin-4-yl)methyl)piperidin-4-yl)piperazin-1-yl)-5-(ethyl(tetrahydro-2H-pyran-4-yl)amino)-4-methyl-[1,1′-biphenyl]-3-carboxamide (GT-08524)

With reference to the method of Scheme T-3, the target compound (GT-08524) was obtained (yellow solid, 28 mg, yield 43%). ¹H NMR (400 MHz, DMSO-d₆) δ 11.84 (s, 2H), 10.84 (s, 1H), 10.46 (s, 1H), 8.46 (s, 1H), 7.73 (d, J=8.6 Hz, 3H), 7.60 (s, 1H), 7.41 (s, 1H), 7.32 (d, J=8.9 Hz, 1H), 7.12 (d, J=8.6 Hz, 2H), 5.91 (s, 1H), 4.31 (d, J=4.8 Hz, 2H), 4.12 (d, J=12.9 Hz, 2H), 3.96 (d, J=10.7 Hz, 2H), 3.78 (s, 5H), 3.69 (s, 2H), 3.57 (d, J=9.3 Hz, 2H), 3.36-3.18 (m, 6H), 3.07-2.93 (m, 5H), 2.87-2.78 (m, 2H), 2.36 (d, J=22.3 Hz, 5H), 2.24 (s, 3H), 2.21-2.17 (m, 1H), 2.13 (s, 3H), 1.95 (d, J=10.8 Hz, 2H), 1.66 (d, J=55.0 Hz, 2H), 1.27 (d, J=11.3 Hz, 2H), 0.93 (s, 2H). LCMS (ESI) calcd, for C₅₆H₇₁N₁₀O₇⁺ [M+H]⁺: 995.55, found, 995.4.

### Example 343: Preparation of N-((4,6-dimethyl-2-oxo-1,2-dihydropyridin-3-yl)methyl)-4′-(4-(1-((1-(2-(2,4-dioxotetrahydropyrimidin-1(2H)-yl)-1,3-dioxoisoindolin-4-yl)piperidin-4-yl)methyl)piperidin-4-yl)piperazin-1-yl)-5-(ethyl(tetrahydro-2H-pyran-4-yl)amino)-4-methyl-[1,1′-biphenyl]-3-carboxamide (GT-08525)

With reference to the method of Scheme T-3, the target compound (GT-08525) was obtained (yellow solid, 25 mg, yield 38%). ¹H NMR (400 MHz, DMSO-d₆) δ 11.81 (s, 2H), 10.86 (s, 1H), 10.47 (s, 1H), 8.46 (s, 1H), 8.02 (s, 1H), 7.77-7.71 (m, 2H), 7.60 (s, 1H), 7.41 (t, J=7.7 Hz, 2H), 7.12 (d, J=8.4 Hz, 2H), 5.91 (s, 1H), 4.31 (d, J=4.8 Hz, 2H), 4.03 (s, 1H), 3.96 (d, J=10.1 Hz, 3H), 3.80 (s, 6H), 3.75-3.67 (m, 8H), 3.57 (d, J=8.5 Hz, 4H), 3.37-3.20 (m, 6H), 3.06-2.94 (m, 4H), 2.87-2.78 (m, 2H), 2.47-2.32 (m, 6H), 2.24 (s, 3H), 2.13 (s, 3H), 2.05 (d, J=24.3 Hz, 3H), 1.65 (d, J=52.3 Hz, 2H), 1.52-1.37 (m, 2H), 0.93 (s, 2H). LCMS (ESI) calcd, for C₅₆H₇₁N₁₀O₇⁺ [M+H]⁺: 995.55, found, 995.4.

### Example 344: Preparation of 5-(4-((1-(4-((5-chloro-4-((2-(dimethylphosphoryl)phenyl)amino)pyrimidin-2-yl)amino)-3-methoxyphenyl)piperidin-4-yl)methyl)piperazin-1-yl)-2-(2,4-dioxotetrahydropyrimidin-1(2H)-yl)isoindoline-1,3-dione (GT-08526)

With reference to the method of Scheme T-3, the target compound (GT-08526) was obtained (white solid, 19 mg, yield 37%). ¹H NMR (400 MHz, DMSO-d₆) δ 11.68 (s, 1H), 11.13 (s, 1H), 10.86 (s, 1H), 8.97 (s, 1H), 8.41 (s, 1H), 8.27 (s, 1H), 7.83 (d, J=8.5 Hz, 2H), 7.66-7.60 (m, 1H), 7.57 (s, 1H), 7.53-7.47 (m, 1H), 7.42 (d, J=8.5 Hz, 2H), 7.24 (t, J=7.3 Hz, 2H), 4.25 (d, J=12.3 Hz, 2H), 3.86 (s, 3H), 3.80 (t, J=6.8 Hz, 3H), 3.54-3.28 (m, 8H), 3.18 (s, 4H), 2.84 (t, J=6.9 Hz, 2H), 2.33-2.25 (m, 1H), 2.16 (d, 2H), 1.83 (s, 1H), 1.81 (s, 3H), 1.78 (s, 3H). LCMS (ESI) calcd, for C₄₁H₄₇ClN₁₀O₆P⁺[M+H]⁺: 841.31, found, 841.2.

### Example 345: Preparation of 5-((1S,4S)-5-((1-(4-((5-chloro-4-((2-(dimethylphosphoryl)phenyl)amino)pyrimidin-2-yl)amino)-3-methoxyphenyl)piperidin-4-yl)methyl)-2,5-diazabicyclo[2.2.1]heptan-2-yl)-2-(2,4-dioxotetrahydropyrimidin-1(2H)-yl)isoindoline-1,3-dione (GT-08527)

With reference to the method of Scheme T-3, the target compound (GT-08527) was obtained (white solid, 27 mg, yield 53%). ¹H NMR (400 MHz, DMSO-d₆) δ 11.68 (d, J=47.3 Hz, 1H), 10.85 (s, 1H), 10.52 (s, 1H), 9.12 (s, 1H), 8.38 (s, 1H), 8.30 (s, 1H), 7.77 (t, J=8.3 Hz, 2H), 7.69-7.52 (m, 2H), 7.49 (t, J=7.3 Hz, 1H), 7.32-7.11 (m, 3H), 7.02 (s, 1H), 5.02 (d, J=20.3 Hz, 1H), 4.65 (d, J=48.3 Hz, 1H), 4.14 (d, J=11.5 Hz, 1H), 3.86 (s, 4H), 3.80-3.78 (m, 3H), 3.76-3.75 (m, 2H), 3.67-3.63 (m, 4H), 3.39-3.30 (m, 2H), 3.25-3.07 (m, 2H), 2.84 (t, J=6.7 Hz, 2H), 2.29-2.11 (m, 4H), 1.81 (s, 3H), 1.77 (s, 3H). LCMS (ESI) calcd. for C₄₂H₄₇ClN₁₀O₆P⁺[M+H]⁺: 853.31, found, 853.2.

### Example 346: Preparation of 5-((1R,4R)-5-((1-(4-((5-chloro-4-((2-(dimethylphosphoryl)phenyl)amino)pyrimidin-2-yl)amino)-3-methoxyphenyl)piperidin-4-yl)methyl)-2,5-diazabicyclo[2.2.1]heptan-2-yl)-2-(2,4-dioxotetrahydropyrimidin-1(2H)-yl)isoindoline-1,3-dione (GT-08528)

With reference to the method of Scheme T-3, the target compound (GT-08528) was obtained (white solid, 19 mg, yield 37%). ¹H NMR (400 MHz, DMSO-d₆) δ 11.63 (d, J=45.4 Hz, 1H), 10.85 (s, 1H), 10.45 (s, 1H), 9.00 (s, 1H), 8.39 (s, 1H), 8.27 (s, 1H), 7.95-7.66 (m, 2H), 7.66-7.57 (m, 1H), 7.49 (t, J=7.4 Hz, 1H), 7.45-7.11 (m, 3H), 7.04 (s, 1H), 5.02 (d, J=19.7 Hz, 1H), 4.65 (d, J=48.9 Hz, 1H), 4.12 (d, J=11.1 Hz, 1H), 3.85 (s, 3H), 3.82-3.77 (m, 3H), 3.73 (s, 2H), 3.70 (s, 2H), 3.54-3.44 (m, 3H), 3.39-3.28 (m, 2H), 3.25-3.16 (m, 1H), 3.15-2.96 (m, 1H), 2.84 (t, J=6.7 Hz, 2H), 2.28-2.09 (m, 3H), 1.84 (s, 1H), 1.79 (d, J=13.6 Hz, 6H). LCMS (ESI) calcd, for C₄₂H₄₇ClN₁₀O₆P⁺[M+H]⁺: 853.31, found, 853.2.

### Example 347: Preparation of 5-(4-((1-(4-((5-chloro-4-((2-(dimethylphosphoryl)phenyl)amino)pyrimidin-2-yl)amino)-3-methoxyphenyl)piperidin-4-yl)methyl)piperazin-1-yl)-2-(2,4-dioxotetrahydropyrimidin-1(2H)-yl)-6-fluoroisoindoline-1,3-dione (GT-08529)

With reference to the method of Scheme T-3, the target compound (GT-08529) was obtained (white solid, 5 mg, yield 10%). ¹H NMR (400 MHz, DMSO-d₆) δ 11.59 (s, 1H), 10.89 (s, 1H), 10.83 (s, 1H), 8.91 (s, 1H), 8.34 (s, 1H), 8.19 (s, 1H), 7.84 (d, J=11.4 Hz, 1H), 7.63 (s, 1H), 7.63-7.59 (m, 1H), 7.59-7.53 (m, 1H), 7.42 (t, J=7.7 Hz, 1H), 7.17 (t, J=7.1 Hz, 2H), 3.79 (s, 3H), 3.74 (t, J=6.0 Hz, 3H), 3.61-3.54 (m, 6H), 3.50 (s, 2H), 3.23-3.16 (m, 4H), 3.14 (s, 2H), 2.78 (t, J=6.7 Hz, 2H), 2.24-2.15 (m, 1H), 2.07 (d, 2H), 1.74 (s, 3H), 1.71 (s, 3H). LCMS (ESI) calcd, for C₄₁H₄₆ClFN₁₀O₆P⁺ [M+H]⁺: 859.30, found, 859.1.

### Example 348: Preparation of 5-(3-((1-(4-((5-chloro-4-((2-(dimethylphosphoryl)phenyl)amino)pyrimidin-2-yl)amino)-3-methoxyphenyl)piperidin-4-yl)methyl)-3,8-diazabicyclo[3.2.1]octan-8-yl)-2-(2,4-dioxotetrahydropyrimidin-1(2H)-yl)isoindoline-1,3-dione (GT-08530)

With reference to the method of Scheme T-3, the target compound (GT-08530) was obtained (white solid, 7 mg, yield 14%). ¹H NMR (400 MHz, DMSO-d₆) δ 11.73 (s, 1H), 10.86 (s, 2H), 9.19 (s, 1H), 8.32 (s, 2H), 7.86-7.73 (m, 2H), 7.63 (d, 1H), 7.56-7.43 (m, 3H), 7.34 (d, 1H), 7.24 (t, J=7.2 Hz, 1H), 4.83 (s, 2H), 3.87 (d, J=6.2 Hz, 3H), 3.79 (t, J=6.9 Hz, 3H), 3.57-3.54 (m, 3H), 3.46-3.39 (m, 3H), 3.14-3.06 (m, 2H), 3.02 (s, 2H), 2.83 (t, J=15.9, 9.2 Hz, 2H), 2.47 (s, 1H), 2.33 (s, 1H), 2.10 (d, J=5.0 Hz, 4H), 1.88 (s, 2H), 1.81 (s, 3H), 1.78 (s, 3H). LCMS (ESI) calcd, for C₄₃H₄₉ClN₁₀O₆P⁺[M+H]⁺: 867.33, found, 867.2.

### Example 349: Preparation of (R)—N-(1-(4-(4-((1-(2-(2,4-dioxotetrahydropyrimidin-1(2H)-yl)-1,3-dioxoisoindolin-5-yl)piperidin-4-yl)methyl)piperazin-1-yl)benzyl)-1H-pyrazol-4-yl)-9-methyl-6-oxo-6,7,8,9-tetrahydropyrido[3′,2′: 4,5]pyrrolo[1,2-a]pyrazine-2-carboxamide (GT-08255)

With reference to the method of Scheme T-3, the target compound (GT-08255) was obtained (yellow solid, 17 mg, yield 25%). ¹H NMR (400 MHz, DMSO-d₆) δ 10.84 (s, 1H), 10.64 (s, 1H), 10.37 (s, 1H), 8.36 (d, J=4.8 Hz, 1H), 8.30 (d, J=8.2 Hz, 1H), 8.18 (s, 1H), 7.91 (d, J=8.2 Hz, 1H), 7.77 (s, 1H), 7.72 (d, J=8.6 Hz, 1H), 7.41 (d, J=1.9 Hz, 1H), 7.36-7.28 (m, 1H), 7.24 (d, J=8.6 Hz, 2H), 7.14 (s, 1H), 7.00 (d, J=8.7 Hz, 2H), 5.32-5.26 (m, 1H), 5.24 (s, 2H), 4.12 (d, J=13.3 Hz, 2H), 3.91 (dd, J=12.9, 4.3 Hz, 1H), 3.81-3.79 (m, 1H), 3.79-3.76 (m, 2H), 3.58-3.55 (m, 3H), 3.50-3.46 (m, 1H), 3.25 (t, J=11.9 Hz, 2H), 3.17-3.10 (m, 2H), 3.09-2.97 (m, 4H), 2.84 (t, 2H), 2.21 (s, 1H), 1.93 (d, J=11.7 Hz, 2H), 1.42 (d, J=6.5 Hz, 3H), 1.35-1.20 (m, 2H). LCMS (ESI) calcd, for C₄₄H₄₇N₁₂O₆⁺ [M+H]⁺: 861.36, found, 861.2.

### Example 350: Preparation of (R)—N-(1-(4-(4-((1-(2-(2,4-dioxotetrahydropyrimidin-1(2H)-yl)-1,3-dioxoisoindolin-4-yl)piperidin-4-yl)methyl)piperazin-1-yl)benzyl)-1H-pyrazol-4-yl)-9-methyl-6-oxo-6,7,8,9-tetrahydropyrido[3′,2′: 4,5]pyrrolo[1,2-a]pyrazine-2-carboxamide (GT-08256)

With reference to the method of Scheme T-3, the target compound (GT-08256) was obtained (yellow solid, 11 mg, yield 16%). ¹H NMR (400 MHz, DMSO-d₆) δ 10.86 (s, 1H), 10.64 (s, 1H), 10.39 (s, 1H), 8.36 (d, J=4.5 Hz, 1H), 8.30 (d, J=8.2 Hz, 1H), 8.20-8.15 (m, 1H), 7.91 (d, J=8.2 Hz, 1H), 7.78 (s, 1H), 7.77-7.71 (m, 1H), 7.39 (t, J=6.9 Hz, 1H), 7.26-7.21 (m, 2H), 7.14 (s, 1H), 7.01 (d, J=8.6 Hz, 1H), 5.33-5.27 (m, 1H), 5.24 (s, 2H), 3.91 (dd, J=12.9, 4.4 Hz, 1H), 3.82-3.76 (m, 4H), 3.76-3.73 (m, 4H), 3.61 (d, J=10.2 Hz, 3H), 3.50-3.44 (m, 1H), 3.33-3.21 (m, 2H), 3.16 (d, J=24.1 Hz, 4H), 3.05-2.86 (m, 2H), 2.85-2.81 (m, 1H), 2.10 (s, 1H), 2.05-1.95 (m, 2H), 1.43 (d, J=6.5 Hz, 3H). LCMS (ESI) calcd, for C₄₄H₄₇N₁₂O₆⁺ [M+H]⁺: 839.37, found, 839.2.

### Example 351: Preparation of (R)—N-(1-(4-(4-(4-((1-(2-(2,4-dioxotetrahydropyrimidin-1(2H)-yl)-1,3-dioxoisoindolin-5-yl)piperidin-4-yl)methyl)piperazin-1-yl)piperidin-1-yl)benzyl)-1H-pyrazol-4-yl)-9-methyl-6-oxo-6,7,8,9-tetrahydropyrido[3′,2′: 4,5]pyrrolo[1,2-a]pyrazine-2-carboxamide (GT-08343)

With reference to the method of Scheme T-3, the target compound (GT-08343) was obtained (yellow solid, 32 mg, yield 50%). ¹H NMR (400 MHz, DMSO-d₆) δ 10.84 (s, 1H), 10.65 (s, 1H), 8.35 (d, J=4.7 Hz, 1H), 8.30 (d, J=8.2 Hz, 1H), 8.19 (s, 1H), 7.91 (d, J=8.2 Hz, 1H), 7.78 (s, 1H), 7.72 (d, J=8.6 Hz, 1H), 7.41 (s, 1H), 7.32 (d, J=8.8 Hz, 1H), 7.24 (d, J=8.6 Hz, 2H), 7.17-7.08 (m, 3H), 5.32-5.27 (m, 1H), 5.25 (s, 2H), 4.11 (d, J=13.0 Hz, 2H), 3.93-3.84 (m, 4H), 3.78 (t, J=6.9 Hz, 3H), 3.73 (s, 4H), 3.52-3.45 (m, 5H), 3.12-2.97 (m, 4H), 2.83 (t, J=7.3 Hz, 4H), 2.20 (d, 3H), 2.00 (d, J=11.0 Hz, 2H), 1.92 (s, 2H), 1.42 (d, J=6.6 Hz, 3H), 1.31-1.21 (m, 2H). LCMS (ESI) calcd, for C₄₉H₅₆N₁₃O₆⁺ [M+H]⁺: 922.45, found, 922.3.

### Example 352: Preparation of (R)—N-(1-(4-(4-(4-((1-(2-(2,4-dioxotetrahydropyrimidin-1(2H)-yl)-1,3-dioxoisoindolin-4-yl)piperidin-4-yl)methyl)piperazin-1-yl)piperidin-1-yl)benzyl)-1H-pyrazol-4-yl)-9-methyl-6-oxo-6,7,8,9-tetrahydropyrido[3′,2′: 4,5]pyrrolo[1,2-a]pyrazine-2-carboxamide (GT-08344)

With reference to the method of Scheme T-3, the target compound (GT-08344) was obtained (yellow solid, 28 mg, yield 44%). ¹H NMR (400 MHz, DMSO-d₆) δ 10.85 (s, 1H), 10.65 (s, 1H), 8.35 (d, J=4.7 Hz, 1H), 8.30 (d, J=8.2 Hz, 1H), 8.19 (s, 1H), 7.91 (d, J=8.2 Hz, 1H), 7.78 (s, 1H), 7.77-7.72 (m, 1H), 7.40 (t, J=7.8 Hz, 2H), 7.25 (d, J=8.4 Hz, 2H), 7.16-7.08 (m, 3H), 5.31-5.27 (m, 1H), 5.25 (s, 2H), 3.94-3.83 (m, 6H), 3.80-3.77 (m, 3H), 3.76-3.73 (m, 4H), 3.51-3.44 (m, 4H), 3.15 (s, 2H), 2.95-2.79 (m, 6H), 2.22 (d, J=10.3 Hz, 2H), 2.06 (s, 3H), 1.97-1.87 (m, 2H), 1.42 (d, J=6.5 Hz, 5H). LCMS (ESI) calcd, for C₄₉H₅₆N₁₃O₆⁺ [M+H]⁺: 922.45, found, 922.3.

### Example 353: Preparation of (R)—N-(1-(4-(4-((2-(2,4-dioxotetrahydropyrimidin-1(2H)-yl)-1-oxoisoindolin-5-yl)methyl)piperazin-1-yl)benzyl)-1H-pyrazol-4-yl)-9-methyl-6-oxo-6,7,8,9-tetrahydropyrido[3′,2′: 4,5]pyrrolo[1,2-a]pyrazine-2-carboxamide (GT-08149)

With reference to the method of Scheme T-3, the target compound (GT-08149) was obtained (white solid, 25 mg, yield 42%). ¹H NMR (400 MHz, DMSO-d₆) δ 11.17 (s, 1H), 10.69 (s, 1H), 10.63 (s, 1H), 8.35 (d, J=4.8 Hz, 1H), 8.30 (d, J=8.2 Hz, 1H), 8.17 (s, 1H), 7.92-7.86 (m, 3H), 7.82-7.76 (m, 2H), 7.23 (d, J=8.6 Hz, 2H), 7.13 (s, 1H), 6.97 (d, J=8.7 Hz, 2H), 5.31-5.25 (m, 1H), 5.23 (s, 2H), 4.81 (d, J=16.4 Hz, 1H), 4.62-4.48 (m, 3H), 3.90 (dd, J=12.9, 4.4 Hz, 1H), 3.85-3.76 (m, 4H), 3.38 (s, 3H), 3.21-3.10 (m, 4H), 2.93-2.81 (m, 1H), 2.81-2.70 (m, 1H), 1.42 (d, J=6.5 Hz, 3H). LCMS (ESI) calcd, for C₃₉H₄₀N₁₁O₅⁺ [M+H]⁺: 742.32, found, 742.2.

### Example 354: Preparation of (R)—N-(1-(4-(4-((2-(2,4-dioxotetrahydropyrimidin-1(2H)-yl)-1,3-dioxoisoindolin-5-yl)methyl)piperazin-1-yl)benzyl)-1H-pyrazol-4-yl)-9-methyl-6-oxo-6,7,8,9-tetrahydropyrido[3′,2′: 4,5]pyrrolo[1,2-a]pyrazine-2-carboxamide (GT-08150)

With reference to the method of Scheme T-3, the target compound (GT-08150) was obtained (white solid, 31 mg, yield 48%). ¹H NMR (400 MHz, DMSO-d₆) δ 11.40 (s, 1H), 10.93 (s, 1H), 10.64 (s, 1H), 8.35 (d, J=4.6 Hz, 1H), 8.32-8.31 (m, 1H), 8.18 (d, J=7.9 Hz, 2H), 8.11 (d, J=7.7 Hz, 1H), 7.91 (d, J=8.3 Hz, 1H), 7.77 (s, 1H), 7.23 (d, J=8.5 Hz, 2H), 7.13 (s, 1H), 6.98 (d, J=8.5 Hz, 2H), 5.31-5.27 (m, 1H), 5.23 (s, 2H), 4.60 (s, 2H), 3.91 (dd, J=12.9, 4.2 Hz, 1H), 3.82 (t, J=6.6 Hz, 3H), 3.49-3.44 (m, 2H), 3.39 (s, 2H), 3.18 (s, 4H), 2.86 (t, J=6.7 Hz, 2H), 1.42 (d, J=6.5 Hz, 3H). LCMS (ESI) calcd, for C₃₉H₃₈N₁₁O₆⁺ [M+H]⁺: 756.30, found, 756.2.

### Example 355: Preparation of (R)—N-(1-(4-(4-(4-((2-(2,4-dioxotetrahydropyrimidin-1(2H)-yl)-1-oxoisoindolin-5-yl)methyl)piperazin-1-yl)piperidin-1-yl)benzyl)-1H-pyrazol-4-yl)-9-methyl-6-oxo-6,7,8,9-tetrahydropyrido[3′,2′: 4,5]pyrrolo[1,2-a]pyrazine-2-carboxamide (GT-08151)

With reference to the method of Scheme T-3, the target compound (GT-08151) was obtained (white solid, 32 mg, yield 57%). ¹H NMR (400 MHz, DMSO-d₆) δ 12.08 (s, 1H), 10.67 (d, J=17.2 Hz, 2H), 8.36 (d, J=4.8 Hz, 1H), 8.30 (d, J=8.2 Hz, 1H), 8.19 (s, 1H), 7.95-7.89 (m, 2H), 7.88-7.81 (m, 2H), 7.78 (s, 1H), 7.25 (d, J=8.6 Hz, 2H), 7.17-7.12 (m, 2H), 5.33-5.24 (m, 3H), 4.80 (d, J=16.6 Hz, 1H), 4.56 (d, J=17.4 Hz, 3H), 3.91 (dd, J=12.9, 4.4 Hz, 2H), 3.86-3.81 (m, 3H), 3.81-3.77 (m, 4H), 3.61-3.59 (m, 3H), 3.49 (d, J=5.2 Hz, 2H), 3.46 (d, J=4.9 Hz, 1H), 2.95-2.83 (m, 3H), 2.79-2.73 (m, 1H), 2.21 (d, J=10.3 Hz, 2H), 1.99-1.85 (m, 2H), 1.42 (d, J=6.5 Hz, 3H). LCMS (ESI) calcd, for C₄₄H₄₉N₁₂O₅⁺ [M+H]⁺: 825.39, found, 825.2.

### Example 356: Preparation of (R)—N-(1-(4-(4-(4-((2-(2,4-dioxotetrahydropyrimidin-1(2H)-yl)-1,3-dioxoisoindolin-5-yl)methyl)piperazin-1-yl)piperidin-1-yl)benzyl)-1H-pyrazol-4-yl)-9-methyl-6-oxo-6,7,8,9-tetrahydropyrido[3′,2′: 4,5]pyrrolo[1,2-a]pyrazine-2-carboxamide (GT-08152)

With reference to the method of Scheme T-3, the target compound (GT-08152) was obtained (white solid, 32 mg, yield 56%). ¹H NMR (400 MHz, DMSO-d₆) δ 10.93 (s, 1H), 10.64 (s, 1H), 8.35 (d, J=4.9 Hz, 1H), 8.30 (d, J=8.2 Hz, 2H), 8.23-8.13 (m, 2H), 8.08 (d, J=7.7 Hz, 1H), 7.91 (d, J=8.2 Hz, 1H), 7.78 (s, 1H), 7.24 (d, J=8.6 Hz, 2H), 7.19-7.08 (m, 3H), 5.35-5.18 (m, 3H), 4.53 (s, 2H), 3.94-3.88 (m, 2H), 3.82 (t, J=6.8 Hz, 6H), 3.57-3.52 (m, 4H), 3.51-3.45 (m, 4H), 2.86 (t, J=6.8 Hz, 3H), 2.20 (d, J=10.4 Hz, 2H), 2.01-1.81 (m, 2H), 1.42 (d, J=6.5 Hz, 3H). LCMS (ESI) calcd, for C₄₄H₄₇N₁₂O₆⁺ [M+H]⁺: 839.37, found, 839.2.

### Example 357: Preparation of 1-(5-((4-(5-(5H-pyrido[4,3-b]indol-7-yl)pyridin-2-yl)piperazin-1-yl)methyl)-1-oxoisoindolin-2-yl)dihydropyrimidine-2,4 (1H,3H)-dione (GT-08535)

With reference to the method of Scheme T-3, the target compound (GT-08535) was obtained (white solid, 16 mg, yield 29%). ¹H NMR (400 MHz, DMSO-d₆) δ 13.38 (s, 1H), 10.70 (s, 1H), 9.79 (s, 1H), 8.67-8.61 (m, 2H), 8.53 (d, J=8.3 Hz, 1H), 8.16 (dd, J=8.9, 2.3 Hz, 1H), 8.03-7.98 (m, 2H), 7.97 (s, 1H), 7.87 (d, J=3.6 Hz, 2H), 7.80 (dd, J=8.4, 1.2 Hz, 1H), 7.16 (d, J=9.0 Hz, 1H), 4.63 (s, 1H), 4.54 (s, 3H), 3.84-3.78 (m, 4H), 3.51 (s, 1H), 3.44 (d, J=9.6 Hz, 3H), 3.17 (d, J=14.2 Hz, 2H), 2.89-2.84 (m, 1H), 2.81-2.76 (m, 1H). LCMS (ESI) calcd, for C₃₃H₃₁N₈O₃⁺ [M+H]⁺: 587.25, found, 587.1.

### Example 358: Preparation of 1-(5-((4-(5-(5-methyl-5H-pyrido[4,3-b]indol-7-yl)pyridin-2-yl)piperazin-1-yl)methyl)-1-oxoisoindolin-2-yl)dihydropyrimidine-2,4 (1H,3H)-dione (GT-08536)

With reference to the method of Scheme T-3, the target compound (GT-08536) was obtained (white solid, 14 mg, yield 25%). ¹H NMR (400 MHz, DMSO-d₆) δ 10.70 (s, 1H), 9.79 (s, 1H), 8.78-8.70 (m, 2H), 8.55 (d, J=8.3 Hz, 1H), 8.26 (dd, J=9.1, 2.4 Hz, 1H), 8.21 (s, 1H), 7.97 (s, 1H), 7.87 (d, J=5.1 Hz, 2H), 7.17 (d, J=9.0 Hz, 1H), 4.83 (d, J=16.6 Hz, 1H), 4.54 (s, 3H), 4.15 (s, 3H), 3.79 (d, J=6.9 Hz, 4H), 3.51 (s, 2H), 3.46 (s, 1H), 3.43 (s, 1H), 3.19 (s, 2H), 2.86 (t, J=7.9 Hz, 1H), 2.82-2.75 (m, 1H). LCMS (ESI) calcd. for C₃₄H₃₃N₈O₃⁺ [M+H]⁺: 601.27, found, 601.2.

### Example 359: Preparation of 1-(1-oxo-5-((4-(5-(5-(2,2,2-trifluoroethyl)-5H-pyrido[4,3-b]indol-7-yl)pyridin-2-yl)piperazin-1-yl)methyl)isoindolin-2-yl)dihydropyrimidine-2,4 (1H,3H)-dione (GT-08537)

With reference to the method of Scheme T-3, the target compound (GT-08537) was obtained (white solid, 44 mg, yield 70%). ¹H NMR (400 MHz, DMSO-d₆) δ 10.71 (s, 1H), 9.91 (s, 1H), 8.88 (d, J=6.8 Hz, 1H), 8.71 (d, J=2.1 Hz, 1H), 8.59 (d, J=8.3 Hz, 1H), 8.45 (s, 1H), 8.39 (d, J=6.8 Hz, 1H), 8.28 (dd, J=9.0, 2.0 Hz, 1H), 7.99 (s, 1H), 7.93 (d, J=8.4 Hz, 1H), 7.88 (s, 2H), 7.24 (d, J=9.1 Hz, 1H), 5.88 (d, J=8.9 Hz, 2H), 4.84 (d, J=16.6 Hz, 1H), 4.55 (s, 4H), 3.81 (t, J=6.8 Hz, 3H), 3.58 (s, 2H), 3.44 (s, 2H), 3.22 (s, 2H), 2.87 (t, J=7.8 Hz, 1H), 2.79 (t, J=5.5 Hz, 1H). LCMS (ESI) calcd, for C₃₅H₃₂F₃N₈₀₃⁺ [M+H]⁺: 669.25, found, 669.2.

### Example 360: Preparation of 1-(5-((4-(5-(5-(difluoromethyl)-5H-pyrido[4,3-b]indol-7-yl)pyridin-2-yl)piperazin-1-yl)methyl)-1-oxoisoindolin-2-yl)dihydropyrimidine-2,4 (1H,3H)-dione (GT-08538)

With reference to the method of Scheme T-3, the target compound (GT-08538) was obtained (white solid, 21 mg, yield 35%). ¹H NMR (400 MHz, DMSO-d₆) δ 10.70 (s, 1H), 9.98 (s, 1H), 8.92 (d, J=6.8 Hz, 1H), 8.69 (d, J=2.3 Hz, 1H), 8.61 (d, J=8.2 Hz, 1H), 8.41 (d, J=6.6 Hz, 1H), 8.32 (s, 1H), 8.19 (dd, J=9.0, 2.4 Hz, 1H), 7.97 (d, J=9.7 Hz, 2H), 7.87 (dd, J=16.8, 8.0 Hz, 2H), 7.16 (d, J=9.0 Hz, 1H), 4.81 (s, 1H), 4.61 (s, 1H), 4.55 (d, J=13.7 Hz, 3H), 3.82 (s, 1H), 3.80 (s, 2H), 3.79 (s, 1H), 3.47 (d, J=12.1 Hz, 3H), 3.17 (s, 2H), 2.86 (t, J=8.0 Hz, 1H), 2.79 (s, 1H). LCMS (ESI) calcd, for C₃₄H₃₁F₂N₈₀₃⁺ [M+H]⁺: 637.25, found, 637.2.

### Example 361: Preparation of 1-(5-((4-(5-(5-methyl-5H-pyrido[4,3-b]indol-7-yl)-3-(trifluoromethyl)pyridin-2-yl)piperazin-1-yl)methyl)-1-oxoisoindolin-2-yl)dihydropyrimidine-2,4 (1H,3H)-dione (GT-08539)

With reference to the method of Scheme T-3, the target compound (GT-08539) was obtained (white solid, 37 mg, yield 59%). ¹H NMR (400 MHz, DMSO-d₆) δ 10.70 (s, 1H), 9.84 (s, 1H), 9.14 (d, J=1.7 Hz, 1H), 8.78 (d, J=6.9 Hz, 1H), 8.61 (d, J=8.2 Hz, 2H), 8.37 (s, 1H), 8.22 (d, J=6.9 Hz, 1H), 8.03-7.96 (m, 2H), 7.89 (d, J=3.9 Hz, 2H), 4.81 (s, 1H), 4.59 (d, J=16.3 Hz, 3H), 4.16 (s, 3H), 3.80 (t, J=6.9 Hz, 3H), 3.64 (s, 4H), 3.50 (s, 1H), 3.25 (s, 2H), 2.87 (s, 1H), 2.79 (s, 1H). LCMS (ESI) calcd, for C₃₅H₃₂F₃N₈₀₃⁺ [M+H]⁺: 669.25, found, 669.2.

### Example 362: Preparation of 1-(5-((4-(3-methyl-5-(5-methyl-5H-pyrido[4,3-b]indol-7-yl)pyridin-2-yl)piperazin-1-yl)methyl)-1-oxoisoindolin-2-yl)dihydropyrimidine-2,4 (1H,3H)-dione (GT-08540)

With reference to the method of Scheme T-3, the target compound (GT-08540) was obtained (white solid, 26 mg, yield 45%). ¹H NMR (400 MHz, DMSO-d₆) δ 10.70 (s, 1H), 9.81 (s, 1H), 8.76 (d, J=6.5 Hz, 1H), 8.68 (s, 1H), 8.56 (s, 1H), 8.21 (dd, J=14.7, 7.8 Hz, 3H), 8.00 (s, 1H), 7.89 (s, 3H), 4.84 (d, J=16.3 Hz, 1H), 4.57 (s, 2H), 4.15 (s, 4H), 3.83-3.78 (m, 3H), 3.70 (d, J=12.5 Hz, 2H), 3.45 (d, J=11.7 Hz, 3H), 3.31 (s, 2H), 2.88 (d, J=7.4 Hz, 1H), 2.79 (s, 1H), 2.39 (s, 3H). LCMS (ESI) calcd, for C₃₅H₃₅N₈O₃⁺ [M+H]⁺: 615.28, found, 615.2.

### Example 363: Preparation of 1-(5-((4-(3-chloro-5-(5-methyl-5H-pyrido[4,3-b]indol-7-yl)pyridin-2-yl)piperazin-1-yl)methyl)-1-oxoisoindolin-2-yl)dihydropyrimidine-2,4 (1H,3H)-dione (GT-08541)

With reference to the method of Scheme T-3, the target compound (GT-08541) was obtained (white solid, 33 mg, yield 55%). ¹H NMR (400 MHz, DMSO-d₆) δ 10.70 (s, 1H), 9.82 (s, 1H), 8.85 (s, 1H), 8.77 (d, J=6.6 Hz, 1H), 8.58 (d, J=8.2 Hz, 1H), 8.47 (s, 1H), 8.32 (s, 1H), 8.23 (s, 1H), 7.98 (s, 1H), 7.95 (d, J=8.3 Hz, 1H), 7.88 (s, 2H), 4.83 (d, J=16.8 Hz, 1H), 4.57 (s, 3H), 4.15 (s, 4H), 3.95 (s, 3H), 3.79 (d, J=6.9 Hz, 3H), 3.29 (s, 3H), 2.86 (s, 1H), 2.78 (s, 1H). LCMS (ESI) calcd, for C₃₄H₃₂ClN₈O₃⁺ [M+H]⁺: 635.23, found, 635.2.

### Example 364: Preparation of 5-((4-(5-(5H-pyrido[4,3-b]indol-7-yl)pyridin-2-yl)piperazin-1-yl)methyl)-2-(2,4-dioxotetrahydropyrimidin-1(2H)-yl)isoindoline-1,3-dione (GT-08542)

With reference to the method of Scheme T-3, the target compound (GT-08542) was obtained (white solid, 8 mg, yield 15%). ¹H NMR (400 MHz, DMSO-d₆) δ 10.94 (s, 1H), 9.78 (s, 1H), 8.67-8.63 (m, 2H), 8.52 (d, J=8.3 Hz, 1H), 8.37 (s, 1H), 8.22 (s, 1H), 8.15 (s, 1H), 8.12 (d, J=7.7 Hz, 1H), 8.01 (d, J=6.7 Hz, 1H), 7.99 (s, 1H), 7.80 (d, J=8.3 Hz, 1H), 7.16 (s, 1H), 4.62 (s, 2H), 4.52 (s, 2H), 3.84 (t, J=6.8 Hz, 3H), 3.46 (d, J=7.0 Hz, 3H), 3.18 (s, 2H), 2.87 (t, J=6.8 Hz, 2H). LCMS (ESI) calcd, for C₃₃H₂₉N₈O₄⁺ [M+H]⁺: 601.23, found, 601.2.

### Example 365: Preparation of 2-(2,4-dioxotetrahydropyrimidin-1(2H)-yl)-5-((4-(5-(5-methyl-5H-pyrido[4,3-b]indol-7-yl)pyridin-2-yl)piperazin-1-yl)methyl)isoindoline-1,3-dione (GT-08543)

With reference to the method of Scheme T-3, the target compound (GT-08543) was obtained (white solid, 13 mg, yield 23%). ¹H NMR (400 MHz, DMSO-d₆) δ 10.94 (s, 1H), 9.79 (s, 1H), 8.75 (d, J=6.9 Hz, 1H), 8.72 (d, J=2.3 Hz, 1H), 8.54 (d, J=8.2 Hz, 1H), 8.39 (s, 1H), 8.30-8.24 (m, 2H), 8.20 (d, J=6.9 Hz, 2H), 8.12 (d, J=7.7 Hz, 1H), 7.89-7.83 (m, 1H), 7.20 (d, J=9.0 Hz, 1H), 4.64 (s, 2H), 4.53 (s, 2H), 4.14 (s, 4H), 3.84 (s, 2H), 3.82 (s, 2H), 3.45 (s, 3H), 3.22 (s, 2H), 2.87 (t, J=6.8 Hz, 2H). LCMS (ESI) calcd, for C₃₄H₃₁N₈O₄⁺ [M+H]⁺: 615.25, found, 615.2.

### Example 366: Preparation of 2-(2,4-dioxotetrahydropyrimidin-1(2H)-yl)-5-((4-(5-(5-(2,2,2-trifluoroethyl)-5H-pyrido[4,3-b]indol-7-yl)pyridin-2-yl)piperazin-1-yl)methyl)isoindoline-1,3-dione (GT-08544)

With reference to the method of Scheme T-3, the target compound (GT-08544) was obtained (white solid, 49 mg, yield 80%). ¹H NMR (400 MHz, DMSO-d₆) δ 10.95 (s, 1H), 9.91 (s, 1H), 8.88 (d, J=6.8 Hz, 1H), 8.71 (d, J=2.3 Hz, 1H), 8.60-8.56 (m, 1H), 8.44 (s, 1H), 8.40 (d, J=7.8 Hz, 2H), 8.27 (d, J=7.8 Hz, 2H), 8.12 (d, J=7.7 Hz, 1H), 7.92 (d, J=8.4 Hz, 1H), 7.23 (d, J=9.1 Hz, 1H), 5.88 (d, J=8.7 Hz, 2H), 4.66 (s, 2H), 4.55 (s, 1H), 3.96 (d, J=4.9 Hz, 2H), 3.46 (s, 2H), 3.24 (s, 3H), 2.88 (t, J=6.8 Hz, 2H), 1.32 (d, J=6.6 Hz, 1H), 1.30-1.26 (m, 1H). LCMS (ESI) calcd, for C₃₅H₃₀F₃N₈O₄⁺ [M+H]⁺: 683.23, found, 683.2.

### Example 367: Preparation of 5-((4-(5-(5-(difluoromethyl)-5H-pyrido[4,3-b]indol-7-yl)pyridin-2-yl)piperazin-1-yl)methyl)-2-(2,4-dioxotetrahydropyrimidin-1(2H)-yl)isoindoline-1,3-dione (GT-08545)

With reference to the method of Scheme T-3, the target compound (GT-08545) was obtained (white solid, 38 mg, yield 65%). ¹H NMR (400 MHz, DMSO-d₆) δ 10.95 (s, 1H), 10.00 (s, 1H), 8.92 (d, J=6.8 Hz, 1H), 8.64 (d, J=2.3 Hz, 1H), 8.60 (d, J=8.3 Hz, 1H), 8.42 (d, J=7.4 Hz, 2H), 8.34 (s, 1H), 8.30-8.22 (m, 2H), 8.12 (d, J=7.7 Hz, 1H), 7.94 (d, J=7.8 Hz, 1H), 7.22 (d, J=9.1 Hz, 1H), 4.67 (s, 2H), 4.56 (s, 1H), 3.85 (t, J=6.7 Hz, 3H), 3.58 (s, 2H), 3.46 (s, 2H), 3.26 (s, 2H), 2.88 (t, J=6.8 Hz, 2H). LCMS (ESI) calcd. for C₃₄H₂₉F₂N₈O₄⁺ [M+H]⁺: 651.23, found, 651.2.

### Example 368: Preparation of 2-(2,4-dioxotetrahydropyrimidin-1(2H)-yl)-5-((4-(5-(5-methyl-5H-pyrido[4,3-b]indol-7-yl)-3-(trifluoromethyl)pyridin-2-yl)piperazin-1-yl)methyl)isoindoline-1,3-dione (GT-08546)

With reference to the method of Scheme T-3, the target compound (GT-08546) was obtained (white solid, 30 mg, yield 49%). ¹H NMR (400 MHz, DMSO-d₆) δ 10.94 (s, 1H), 9.84 (s, 1H), 9.14 (d, J=2.2 Hz, 1H), 8.77 (d, J=6.9 Hz, 1H), 8.63-8.57 (m, 2H), 8.43 (s, 1H), 8.36 (s, 1H), 8.29 (d, J=7.8 Hz, 1H), 8.22 (d, J=6.9 Hz, 1H), 8.12 (d, J=7.7 Hz, 1H), 7.98 (d, J=8.3 Hz, 1H), 4.66 (s, 2H), 4.16 (s, 3H), 3.84 (t, J=6.8 Hz, 2H), 3.64 (s, 5H), 3.44 (s, 2H), 3.30 (s, 2H), 2.87 (t, J=6.8 Hz, 2H). LCMS (ESI) calcd, for C₃₅H₃₀F₃N₈O₄⁺ [M+H]⁺: 683.23, found, 683.2.

### Example 369: Preparation of 2-(2,4-dioxotetrahydropyrimidin-1(2H)-yl)-5-((4-(5-(5-methyl-5H-pyrido[4,3-b]indol-7-yl)-4-(trifluoromethyl)pyridin-2-yl)piperazin-1-yl)methyl)isoindoline-1,3-dione (GT-08547)

With reference to the method of Scheme T-3, the target compound (GT-08547) was obtained (white solid, 36 mg, yield 59%). ¹H NMR (400 MHz, DMSO-d₆) δ 10.96 (s, 1H), 9.88 (s, 1H), 8.79 (d, J=6.9 Hz, 1H), 8.58 (d, J=8.1 Hz, 1H), 8.43 (s, 1H), 8.35 (s, 1H), 8.29 (d, J=8.7 Hz, 1H), 8.25 (d, J=6.9 Hz, 1H), 8.13 (d, J=7.7 Hz, 1H), 7.88 (s, 1H), 7.47 (d, J=8.2 Hz, 1H), 7.36 (d, J=11.3 Hz, 1H), 4.68 (s, 3H), 4.11 (s, 3H), 4.07-3.96 (m, 2H), 3.90 (s, 1H), 3.79-3.74 (m, 1H), 3.59 (s, 2H), 3.46 (s, 1H), 3.21 (s, 2H), 2.88 (t, J=6.8 Hz, 2H). LCMS (ESI) calcd, for C₃₅H₃₀F₃N₈O₄⁺ [M+H]⁺: 683.23, found, 683.2.

### Example 370: Preparation of 2-(2,4-dioxotetrahydropyrimidin-1(2H)-yl)-5-((4-(3-methyl-5-(5-methyl-5H-pyrido[4,3-b]indol-7-yl)pyridin-2-yl)piperazin-1-yl)methyl)isoindoline-1,3-dione (GT-08548)

With reference to the method of Scheme T-3, the target compound (GT-08548) was obtained (white solid, 31 mg, yield 55%). ¹H NMR (400 MHz, DMSO-d₆) δ 10.95 (s, 1H), 9.80 (s, 1H), 8.75 (d, J=6.8 Hz, 1H), 8.67 (d, J=2.1 Hz, 1H), 8.55 (s, 1H), 8.44 (s, 1H), 8.31 (d, J=7.8 Hz, 1H), 8.21 (d, J=3.8 Hz, 2H), 8.19 (s, 1H), 8.13 (d, J=7.7 Hz, 1H), 7.87 (dd, J=8.3, 1.1 Hz, 1H), 4.69 (s, 2H), 4.14 (s, 4H), 3.83 (s, 1H), 3.71 (d, J=11.3 Hz, 2H), 3.49 (s, 2H), 3.44 (s, 2H), 3.36 (s, 2H), 2.87 (s, 2H), 2.39 (s, 3H). LCMS (ESI) calcd. for C₃₅H₃₃N₈O₄⁺ [M+H]⁺: 629.26, found, 629.2.

### Example 371: Preparation of 5-((4-(3-chloro-5-(5-methyl-5H-pyrido[4,3-b]indol-7-yl)pyridin-2-yl)piperazin-1-yl)methyl)-2-(2,4-dioxotetrahydropyrimidin-1(2H)-yl)isoindoline-1,3-dione (GT-08549)

With reference to the method of Scheme T-3, the target compound (GT-08549) was obtained (white solid, 30 mg, yield 51%). ¹H NMR (400 MHz, DMSO-d₆) δ 10.94 (s, 1H), 9.81 (s, 1H), 8.83 (d, J=2.2 Hz, 1H), 8.76 (d, J=6.9 Hz, 1H), 8.56 (d, J=8.2 Hz, 1H), 8.44 (d, J=2.1 Hz, 1H), 8.42 (s, 1H), 8.29 (t, J=3.2 Hz, 2H), 8.21 (d, J=6.9 Hz, 1H), 8.13 (d, J=7.7 Hz, 1H), 7.92 (dd, J=8.3, 1.3 Hz, 1H), 4.67 (s, 2H), 4.14 (s, 3H), 3.96 (d, J=5.9 Hz, 2H), 3.84 (t, J=6.8 Hz, 2H), 3.69-3.59 (m, 1H), 3.48-3.41 (m, 3H), 3.32 (s, 2H), 2.87 (t, J=6.8 Hz, 2H). LCMS (ESI) calcd, for C₃₄H₃₀ClN₈O₄⁺ [M+H]⁺: 649.21, found, 649.2.

### Example 372: Preparation of 2-(2,4-dioxotetrahydropyrimidin-1(2H)-yl)-5-(4-((4-(5-(5-methyl-5H-pyrido[4,3-b]indol-7-yl)pyridin-2-yl)piperazin-1-yl)methyl)piperidin-1-yl)isoindoline-1,3-dione (GT-08550)

With reference to the method of Scheme T-3, the target compound (GT-08550) was obtained (white solid, 32 mg, yield 54%). ¹H NMR (400 MHz, DMSO-d₆) δ 10.85 (s, 1H), 9.80 (s, 1H), 8.75 (d, J=6.9 Hz, 1H), 8.72 (d, J=2.4 Hz, 1H), 8.56 (d, J=8.2 Hz, 1H), 8.30 (s, 1H), 8.26-8.17 (m, 2H), 7.88 (d, J=8.3 Hz, 1H), 7.73 (d, J=8.6 Hz, 1H), 7.42 (s, 1H), 7.33 (d, J=8.8 Hz, 1H), 7.26 (d, J=9.0 Hz, 1H), 4.51 (d, J=13.3 Hz, 2H), 4.15 (s, 4H), 3.67 (s, 3H), 3.07 (d, J=12.5 Hz, 4H), 2.84 (t, J=7.4 Hz, 2H), 2.26 (s, 1H), 2.01 (d, J=11.6 Hz, 2H). LCMS (ESI) calcd, for C₃₉H₄₀N₉O₄⁺ [M+H]⁺: 698.32, found, 698.3.

### Example 373: Preparation of 2-(2,4-dioxotetrahydropyrimidin-1(2H)-yl)-5-(4-((4-(5-(5-(2,2,2-trifluoroethyl)-5H-pyrido[4,3-b]indol-7-yl)pyridin-2-yl)piperazin-1-yl)methyl)piperidin-1-yl)isoindoline-1,3-dione (GT-08551)

With reference to the method of Scheme T-3, the target compound (GT-08551) was obtained (white solid, 44 mg, yield 68%). ¹H NMR (400 MHz, DMSO-d₆) δ 10.85 (s, 1H), 9.91 (s, 1H), 8.88 (d, J=6.9 Hz, 1H), 8.73 (d, J=2.4 Hz, 1H), 8.59 (d, J=8.3 Hz, 1H), 8.45 (s, 1H), 8.39 (d, J=6.8 Hz, 1H), 8.26 (d, J=2.3 Hz, 1H), 7.94 (d, J=9.4 Hz, 1H), 7.73 (d, J=8.6 Hz, 1H), 7.42 (d, J=2.0 Hz, 1H), 7.33 (d, J=8.8 Hz, 1H), 7.26 (d, J=9.0 Hz, 1H), 5.88 (d, J=8.8 Hz, 2H), 4.51 (d, J=13.0 Hz, 2H), 4.13 (d, J=13.2 Hz, 2H), 3.14 (d, J=10.7 Hz, 2H), 3.07 (d, J=11.8 Hz, 4H), 2.84 (t, J=7.4 Hz, 2H), 2.26 (s, 1H), 2.01 (d, J=12.1 Hz, 2H). LCMS (ESI) calcd, for C₄₀H₃₉F₃N₉O₄⁺ [M+H]⁺: 766.31, found, 766.3.

### Example 374: Preparation of 5-(4-((4-(5-(5-(difluoromethyl)-5H-pyrido[4,3-b]indol-7-yl)pyridin-2-yl)piperazin-1-yl)methyl)piperidin-1-yl)-2-(2,4-dioxotetrahydropyrimidin-1(2H)-yl)isoindoline-1,3-dione (GT-08552)

With reference to the method of Scheme T-3, the target compound (GT-08552) was obtained (white solid, 34 mg, yield 55%). ¹H NMR (400 MHz, DMSO-d₆) δ 10.86 (s, 1H), 10.01 (s, 1H), 8.93 (d, J=6.8 Hz, 1H), 8.65 (d, J=2.1 Hz, 1H), 8.62 (d, J=8.3 Hz, 1H), 8.43 (d, J=6.6 Hz, 1H), 8.37 (s, 1H), 8.30 (dd, J=9.0, 2.1 Hz, 1H), 7.97 (d, J=8.4 Hz, 1H), 7.72 (d, J=8.6 Hz, 1H), 7.41 (d, J=1.9 Hz, 1H), 7.31 (s, 2H), 4.54 (d, J=13.3 Hz, 2H), 4.11 (s, 2H), 3.80 (t, J=6.8 Hz, 3H), 3.67 (d, J=11.3 Hz, 2H), 3.09 (s, 2H), 2.87-2.81 (m, 2H), 2.27 (s, 1H), 2.03 (d, J=11.2 Hz, 2H). LCMS (ESI) calcd, for C₃₉H₃₈F₂N₉O₄⁺ [M+H]⁺: 734.30, found, 734.3.

### Example 375: Preparation of 2-(2,4-dioxotetrahydropyrimidin-1(2H)-yl)-5-(4-((4-(5-(5-methyl-5H-pyrido[4,3-b]indol-7-yl)-3-(trifluoromethyl)pyridin-2-yl)piperazin-1-yl)methyl)piperidin-1-yl)isoindoline-1,3-dione (GT-08553)

With reference to the method of Scheme T-3, the target compound (GT-08553) was obtained (white solid, 35 mg, yield 54%). ¹H NMR (400 MHz, DMSO-d₆) δ 10.85 (s, 1H), 9.84 (s, 1H), 9.14 (d, J=2.2 Hz, 1H), 8.77 (d, J=6.9 Hz, 1H), 8.65-8.58 (m, 2H), 8.37 (s, 1H), 8.23 (d, J=6.9 Hz, 1H), 7.99 (dd, J=8.3, 1.3 Hz, 1H), 7.72 (d, J=8.6 Hz, 1H), 7.42 (d, J=2.0 Hz, 1H), 7.32 (dd, J=8.8, 2.1 Hz, 1H), 4.17 (s, 3H), 3.79 (t, J=6.8 Hz, 2H), 3.69 (s, 3H), 3.19 (d, J=8.5 Hz, 1H), 3.17-3.17 (m, 1H), 3.12 (s, 2H), 3.04 (t, J=11.9 Hz, 2H), 2.88-2.80 (m, 2H), 2.26 (s, 1H), 2.02 (d, J=11.8 Hz, 2H). LCMS (ESI) calcd, for C₄₀H₃₉F₃N₉O₄⁺ [M+H]⁺: 766.31, found, 766.3.

### Example 376: Preparation of 2-(2,4-dioxotetrahydropyrimidin-1(2H)-yl)-5-(4-((4-(5-(5-methyl-5H-pyrido[4,3-b]indol-7-yl)-4-(trifluoromethyl)pyridin-2-yl)piperazin-1-yl)methyl)piperidin-1-yl)isoindoline-1,3-dione (GT-08554)

With reference to the method of Scheme T-3, the target compound (GT-08554) was obtained (white solid, 45 mg, yield 69%). ¹H NMR (400 MHz, DMSO-d₆) δ 10.86 (s, 1H), 9.87 (s, 1H), 8.79 (d, J=6.9 Hz, 1H), 8.58 (d, J=8.1 Hz, 1H), 8.35 (s, 1H), 8.25 (d, J=6.9 Hz, 1H), 7.89 (s, 1H), 7.73 (d, J=8.6 Hz, 1H), 7.48 (d, J=8.2 Hz, 1H), 7.41 (d, J=10.0 Hz, 2H), 7.33 (d, J=8.8 Hz, 1H), 4.56 (d, J=13.4 Hz, 2H), 4.10 (s, 3H), 3.80 (t, J=6.8 Hz, 2H), 3.72 (s, 2H), 3.09 (s, 3H), 3.06 (s, 1H), 2.84 (t, J=7.4 Hz, 2H), 2.27 (s, 1H), 2.03 (d, J=11.2 Hz, 2H). LCMS (ESI) calcd, for C₄₀H₃₉F₃N₉O₄⁺ [M+H]⁺: 766.31, found, 766.3.

### Example 377: Preparation of 2-(2,4-dioxotetrahydropyrimidin-1(2H)-yl)-5-(4-((4-(3-methyl-5-(5-methyl-5H-pyrido[4,3-b]indol-7-yl)pyridin-2-yl)piperazin-1-yl)methyl)piperidin-1-yl)isoindoline-1,3-dione (GT-08555)

With reference to the method of Scheme T-3, the target compound (GT-08555) was obtained (white solid, 22 mg, yield 36%). ¹H NMR (400 MHz, DMSO-d₆) δ 10.85 (s, 1H), 9.82 (s, 1H), 8.76 (d, J=6.9 Hz, 1H), 8.69 (d, J=2.0 Hz, 1H), 8.58 (d, J=8.2 Hz, 1H), 8.22 (t, J=9.3 Hz, 3H), 7.89 (d, J=8.6 Hz, 1H), 7.73 (d, J=8.6 Hz, 1H), 7.42 (d, J=1.9 Hz, 1H), 7.33 (dd, J=8.8, 2.0 Hz, 1H), 4.15 (s, 4H), 3.79 (t, J=6.8 Hz, 3H), 3.69 (s, 2H), 3.63 (d, J=11.4 Hz, 3H), 3.26 (d, J=9.7 Hz, 2H), 3.11 (s, 1H), 3.06 (s, 1H), 2.84 (d, J=1.5 Hz, 2H), 2.27 (s, 1H), 2.03 (d, J=11.1 Hz, 2H). LCMS (ESI) calcd, for C₄₀H₄₂N₉O₄⁺ [M+H]⁺: 712.34, found, 712.2.

### Example 378: Preparation of 5-(4-((4-(3-chloro-5-(5-methyl-5H-pyrido[4,3-b]indol-7-yl)pyridin-2-yl)piperazin-1-yl)methyl)piperidin-1-yl)-2-(2,4-dioxotetrahydropyrimidin-1(2H)-yl)isoindoline-1,3-dione (GT-08556)

With reference to the method of Scheme T-3, the target compound (GT-08556) was obtained (white solid, 19 mg, yield 31%). ¹H NMR (400 MHz, DMSO-d₆) δ 10.85 (s, 1H), 9.82 (s, 1H), 8.85 (d, J=2.1 Hz, 1H), 8.76 (s, 1H), 8.58 (d, J=8.2 Hz, 1H), 8.46 (d, J=2.1 Hz, 1H), 8.31 (s, 1H), 8.21 (d, J=6.9 Hz, 1H), 7.94 (dd, J=8.3, 1.2 Hz, 1H), 7.73 (d, J=8.6 Hz, 1H), 7.42 (d, J=2.0 Hz, 1H), 7.32 (dd, J=8.8, 2.1 Hz, 1H), 4.15 (s, 3H), 3.94 (d, J=12.7 Hz, 2H), 3.79 (t, J=6.8 Hz, 3H), 3.27-3.19 (m, 2H), 3.11 (s, 2H), 3.05 (s, 1H), 2.84 (d, J=1.5 Hz, 2H), 2.26 (s, 1H), 2.02 (d, J=11.9 Hz, 2H). LCMS (ESI) calcd, for C₃₉H₃₉ClN₉O₄⁺ [M+H]⁺: 732.28, found, 732.2.

### Example 379: Preparation of 4-(4-((4-(5-(5H-pyrido[4,3-b]indol-7-yl)pyridin-2-yl)piperazin-1-yl)methyl)piperidin-1-yl)-2-(2,4-dioxotetrahydropyrimidin-1(2H)-yl)isoindoline-1,3-dione (GT-08557)

With reference to the method of Scheme T-3, the target compound (GT-08557) was obtained (white solid, 22 mg, yield 38%). ¹H NMR (400 MHz, DMSO-d₆) δ 13.42 (s, 1H), 10.86 (s, 1H), 9.80 (s, 1H), 8.65 (d, J=6.8 Hz, 1H), 8.63 (s, 1H), 8.53 (s, 1H), 8.22 (d, J=9.0 Hz, 1H), 8.02 (d, J=6.5 Hz, 2H), 7.82 (s, 1H), 7.75 (s, 1H), 7.40 (d, J=7.1 Hz, 2H), 7.25 (d, J=9.1 Hz, 1H), 4.50 (d, J=13.5 Hz, 2H), 3.15 (s, 4H), 3.01-2.93 (m, 2H), 2.84 (d, J=2.2 Hz, 2H), 2.15 (s, 1H), 2.05 (d, J=9.6 Hz, 2H), 1.48 (dd, J=19.8, 9.2 Hz, 2H). LCMS (ESI) calcd, for C₃₈H₃₈N₉O₄⁺ [M+H]⁺: 684.30, found, 684.2.

### Example 380: Preparation of 2-(2,4-dioxotetrahydropyrimidin-1(2H)-yl)-4-(4-((4-(5-(5-methyl-5H-pyrido[4,3-b]indol-7-yl)pyridin-2-yl)piperazin-1-yl)methyl)piperidin-1-yl)isoindoline-1,3-dione (GT-08558)

With reference to the method of Scheme T-3, the target compound (GT-08558) was obtained (white solid, 37 mg, yield 62%). ¹H NMR (400 MHz, DMSO-d₆) δ 10.87 (s, 1H), 9.80 (s, 1H), 8.75 (d, J=6.9 Hz, 1H), 8.70 (d, J=2.4 Hz, 1H), 8.55 (d, J=8.2 Hz, 1H), 8.33 (dd, J=9.0, 2.1 Hz, 1H), 8.24-8.17 (m, 2H), 7.87 (d, J=8.3 Hz, 1H), 7.78-7.72 (m, 1H), 7.42 (d, J=8.5 Hz, 1H), 7.39 (d, J=7.1 Hz, 1H), 7.29 (d, J=9.1 Hz, 1H), 4.53 (d, J=12.9 Hz, 2H), 4.15 (s, 3H), 3.20 (s, 1H), 3.15 (s, 3H), 2.97 (dt, J=19.0, 11.2 Hz, 2H), 2.84 (s, 2H), 2.16 (s, 1H), 2.08 (s, 2H), 1.48 (d, J=10.6 Hz, 2H). LCMS (ESI) calcd, for C₃₉H₄₀N₉O₄⁺ [M+H]⁺: 698.32, found, 698.2.

### Example 381: Preparation of 2-(2,4-dioxotetrahydropyrimidin-1(2H)-yl)-4-(4-((4-(5-(5-(2,2,2-trifluoroethyl)-5H-pyrido[4,3-b]indol-7-yl)pyridin-2-yl)piperazin-1-yl)methyl)piperidin-1-yl)isoindoline-1,3-dione (GT-08559)

With reference to the method of Scheme T-3, the target compound (GT-08559) was obtained (white solid, 48 mg, yield 74%). ¹H NMR (400 MHz, DMSO-d₆) δ 10.87 (s, 1H), 9.92 (s, 1H), 8.88 (d, J=6.9 Hz, 1H), 8.72 (d, J=2.3 Hz, 1H), 8.60 (d, J=8.3 Hz, 1H), 8.46 (s, 1H), 8.40 (d, J=6.8 Hz, 1H), 8.30 (d, J=6.8 Hz, 1H), 7.94 (d, J=8.3 Hz, 1H), 7.80-7.71 (m, 1H), 7.43 (d, J=8.6 Hz, 1H), 7.40 (d, J=7.1 Hz, 1H), 7.29 (d, J=9.1 Hz, 1H), 5.89 (d, J=8.8 Hz, 2H), 4.53 (d, J=12.8 Hz, 2H), 3.17 (d, J=16.3 Hz, 4H), 2.97 (d, J=18.5 Hz, 2H), 2.85 (t, J=7.6 Hz, 2H), 2.16 (s, 1H), 2.08 (s, 2H), 1.57-1.40 (m, 2H). LCMS (ESI) calcd, for C₄₀H₃₉F₃N₉O₄⁺[M+H]⁺: 766.31, found, 766.2.

### Example 382: Preparation of 4-(4-((4-(5-(5-(difluoromethyl)-5H-pyrido[4,3-b]indol-7-yl)pyridin-2-yl)piperazin-1-yl)methyl)piperidin-1-yl)-2-(2,4-dioxotetrahydropyrimidin-1(2H)-yl)isoindoline-1,3-dione (GT-08560)

With reference to the method of Scheme T-3, the target compound (GT-08560) was obtained (white solid, 31 mg, yield 50%). ¹H NMR (400 MHz, DMSO-d₆) δ 10.87 (s, 1H), 10.01 (s, 1H), 8.93 (d, J=6.8 Hz, 1H), 8.68 (d, J=2.5 Hz, 1H), 8.63 (d, J=8.3 Hz, 1H), 8.43 (d, J=6.6 Hz, 1H), 8.37 (s, 1H), 8.27 (dd, J=9.0, 2.3 Hz, 1H), 7.98 (dd, J=8.4, 1.1 Hz, 1H), 7.76 (d, J=1.2 Hz, 1H), 7.43 (d, J=8.6 Hz, 1H), 7.40 (d, J=7.1 Hz, 1H), 7.27 (d, J=9.1 Hz, 1H), 4.50 (s, 2H), 3.15 (s, 4H), 2.97 (dt, J=19.2, 11.3 Hz, 2H), 2.92-2.80 (m, 2H), 2.16 (s, 1H), 2.07 (s, 2H), 1.56-1.45 (m, 2H). LCMS (ESI) calcd, for C₃₉H₃₈F₂N₉O₄⁺ [M+H]⁺: 734.30, found, 734.2.

### Example 383: Preparation of 2-(2,4-dioxotetrahydropyrimidin-1(2H)-yl)-4-(4-((4-(5-(5-methyl-5H-pyrido[4,3-b]indol-7-yl)-3-(trifluoromethyl)pyridin-2-yl)piperazin-1-yl)methyl)piperidin-1-yl)isoindoline-1,3-dione (GT-08561)

With reference to the method of Scheme T-3, the target compound (GT-08561) was obtained (white solid, 34 mg, yield 52%). ¹H NMR (400 MHz, DMSO-d₆) δ 10.87 (s, 1H), 9.84 (s, 1H), 9.15 (d, J=2.1 Hz, 1H), 8.78 (d, J=6.9 Hz, 1H), 8.62 (dd, J=5.2, 2.9 Hz, 2H), 8.38 (s, 1H), 8.23 (d, J=6.9 Hz, 1H), 8.00 (dd, J=8.3, 1.2 Hz, 1H), 7.76 (dd, J=8.3, 7.3 Hz, 1H), 7.41 (dd, J=11.0, 7.8 Hz, 2H), 4.17 (s, 3H), 3.82-3.78 (m, 2H), 3.18 (s, 4H), 2.96 (dt, J=18.9, 11.3 Hz, 2H), 2.84 (d, J=2.4 Hz, 2H), 2.15 (s, 1H), 2.06 (s, 2H), 1.58-1.40 (m, 2H). LCMS (ESI) calcd, for C₄₀H₃₉F₃N₉O₄⁺ [M+H]⁺: 766.31, found, 766.2.

### Example 384: Preparation of 2-(2,4-dioxotetrahydropyrimidin-1(2H)-yl)-4-(4-((4-(5-(5-methyl-5H-pyrido[4,3-b]indol-7-yl)-4-(trifluoromethyl)pyridin-2-yl)piperazin-1-yl)methyl)piperidin-1-yl)isoindoline-1,3-dione (GT-08562)

With reference to the method of Scheme T-3, the target compound (GT-08562) was obtained (white solid, 45 mg, yield 69%). ¹H NMR (400 MHz, DMSO-d₆) δ 10.88 (s, 1H), 9.88 (s, 1H), 8.79 (d, J=6.9 Hz, 1H), 8.59 (d, J=8.1 Hz, 1H), 8.36 (s, 1H), 8.25 (d, J=6.9 Hz, 1H), 7.89 (s, 1H), 7.76 (d, J=1.2 Hz, 1H), 7.48 (d, J=8.2 Hz, 1H), 7.43 (d, J=8.6 Hz, 1H), 7.41-7.38 (m, 2H), 4.57 (d, J=13.3 Hz, 2H), 4.11 (s, 3H), 3.16 (s, 4H), 2.97 (dt, J=19.3, 11.4 Hz, 2H), 2.85 (s, 2H), 2.17 (s, 1H), 2.10 (t, J=11.9 Hz, 2H), 1.54-1.46 (m, 2H). LCMS (ESI) calcd, for C₄₀H₃₉F₃N₉O₄⁺ [M+H]⁺: 766.31, found, 766.2.

### Example 385: Preparation of 2-(2,4-dioxotetrahydropyrimidin-1(2H)-yl)-4-(4-((4-(3-methyl-5-(5-methyl-5H-pyrido[4,3-b]indol-7-yl)pyridin-2-yl)piperazin-1-yl)methyl)piperidin-1-yl)isoindoline-1,3-dione (GT-08563)

With reference to the method of Scheme T-3, the target compound (GT-08563) was obtained (white solid, 10 mg, yield 17%). ¹H NMR (400 MHz, DMSO-d₆) δ 10.86 (s, 1H), 9.81 (s, 1H), 8.77 (d, J=6.9 Hz, 1H), 8.71 (d, J=2.2 Hz, 1H), 8.58 (d, J=8.2 Hz, 1H), 8.26 (s, 1H), 8.22 (d, J=6.9 Hz, 1H), 8.17 (d, J=2.0 Hz, 1H), 7.91 (dd, J=8.3, 1.2 Hz, 1H), 7.76 (dd, J=8.4, 7.2 Hz, 1H), 7.43 (d, J=8.6 Hz, 1H), 7.40 (d, J=7.1 Hz, 1H), 4.16 (s, 3H), 3.80 (d, J=6.8 Hz, 2H), 3.25 (d, J=9.5 Hz, 2H), 3.17 (s, 2H), 3.03-2.90 (m, 2H), 2.84 (d, J=4.9 Hz, 2H), 2.41 (s, 3H), 2.15 (s, 1H), 2.05 (s, 2H), 1.60-1.41 (m, 2H). LCMS (ESI) calcd, for C₄₀H₄₂N₉O₄⁺ [M+H]⁺: 712.34, found, 712.2.

### Example 386: Preparation of 4-(4-((4-(3-chloro-5-(5-methyl-5H-pyrido[4,3-b]indol-7-yl)pyridin-2-yl)piperazin-1-yl)methyl)piperidin-1-yl)-2-(2,4-dioxotetrahydropyrimidin-1(2H)-yl)isoindoline-1,3-dione (GT-08564)

With reference to the method of Scheme T-3, the target compound (GT-08564) was obtained (white solid, 15 mg, yield 24%). ¹H NMR (400 MHz, DMSO-d₆) δ 10.86 (s, 1H), 9.82 (s, 1H), 8.86 (d, J=2.1 Hz, 1H), 8.77 (d, J=6.9 Hz, 1H), 8.59 (d, J=8.2 Hz, 1H), 8.48 (d, J=2.1 Hz, 1H), 8.33 (s, 1H), 8.22 (d, J=6.9 Hz, 1H), 7.96 (d, J=8.4 Hz, 1H), 7.78-7.73 (m, 1H), 7.41 (dd, J=10.7, 7.8 Hz, 2H), 4.16 (s, 3H), 3.96 (d, J=12.9 Hz, 2H), 3.82-3.77 (m, 2H), 3.68 (d, J=11.2 Hz, 4H), 3.62 (d, J=13.8 Hz, 2H), 3.24 (d, J=11.1 Hz, 2H), 3.17 (s, 2H), 3.03-2.90 (m, 2H), 2.84 (s, 2H), 2.15 (s, 1H), 2.06 (s, 2H), 1.51 (dd, J=24.5, 15.3 Hz, 2H). LCMS (ESI) calcd, for C₃₉H₃₉ClN₉O₄⁺ [M+H]⁺: 732.28, found, 732.2.

### Example 387: Preparation of 1-(5-((4-(benzo[4,5]imidazo[1,2-a]pyrimidin-2-yl)piperazin-1-yl)methyl)-1-oxoisoindolin-2-yl)dihydropyrimidine-2,4 (1H,3H)-dione (GT-08565)

With reference to the method of Scheme T-3, the target compound (GT-08565) was obtained (white solid, 28 mg, yield 58%). ¹H NMR (400 MHz, DMSO-d₆) δ 10.70 (s, 1H), 9.45 (d, J=7.9 Hz, 1H), 8.28 (d, J=8.1 Hz, 1H), 7.96 (s, 1H), 7.86 (d, J=4.1 Hz, 2H), 7.67 (d, J=7.9 Hz, 1H), 7.59 (s, 1H), 7.52 (s, 1H), 7.42 (d, J=7.9 Hz, 1H), 4.81 (t, J=16.7 Hz, 2H), 4.58 (d, J=16.6 Hz, 2H), 4.52 (s, 2H), 3.80 (t, J=6.8 Hz, 3H), 3.65-3.56 (m, 1H), 3.34-3.22 (m, 3H), 3.19-3.10 (m, 1H), 2.86 (t, J=7.8 Hz, 1H), 2.78 (t, J=5.5 Hz, 1H). LCMS (ESI) calcd, for C₂₇H₂₇N₈O₃⁺ [M+H]⁺: 511.22, found, 511.2.

### Example 388: Preparation of 1-(5-(((1-(4-(4-acetyl-1,4-diazepan-1-yl)-6-((2-(thiophen-2-yl)ethyl)amino)-1,3,5-triazin-2-yl)piperidin-4-yl)(methyl)amino)methyl)-1-oxoisoindolin-2-yl)dihydropyrimidine-2,4 (1H,3H)-dione (GT-08566)

With reference to the method of Scheme T-3, the target compound (GT-08566) was obtained (white solid, 32 mg, yield 48%). ¹H NMR (400 MHz, DMSO-d₆) δ 10.69 (s, 1H), 9.34 (s, 3H), 8.74-8.28 (m, 2H), 7.99 (s, 1H), 7.87 (s, 1H), 7.86 (s, 1H), 7.37 (s, 2H), 7.04-6.89 (m, 5H), 4.81 (d, J=13.8 Hz, 2H), 4.68 (s, 2H), 4.58 (d, J=16.9 Hz, 3H), 4.37 (d, J=6.2 Hz, 1H), 3.92 (s, 3H), 3.28 (s, 3H), 3.01 (d, J=10.7 Hz, 2H), 2.87 (d, J=7.8 Hz, 1H), 2.77 (d, J=5.4 Hz, 1H), 2.14 (d, J=11.1 Hz, 3H), 2.01 (s, 4H), 1.97 (d, J=5.0 Hz, 3H), 1.88 (d, J=26.9 Hz, 4H), 1.74 (s, 3H), 1.56 (s, 2H). LCMS (ESI) calcd, for C₃₅H₄₆N₁₁O₄S⁺ [M+H]⁺: 716.34, found, 716.2.

### Example 389: Preparation of 5-((4-(benzo[4,5]imidazo[1,2-a]pyrimidin-2-yl)piperazin-1-yl)methyl)-2-(2,4-dioxotetrahydropyrimidin-1(2H)-yl)isoindoline-1,3-dione (GT-08567)

With reference to the method of Scheme T-3, the target compound (GT-08567) was obtained (white solid, 23 mg, yield 48%). ¹H NMR (400 MHz, DMSO-d₆) δ 10.93 (s, 1H), 9.45 (d, J=7.9 Hz, 1H), 8.36 (s, 1H), 8.28 (d, J=8.1 Hz, 1H), 8.23 (d, J=7.1 Hz, 1H), 8.11 (d, J=7.7 Hz, 1H), 7.67 (d, J=8.0 Hz, 1H), 7.59 (s, 1H), 7.53 (d, J=8.2 Hz, 1H), 7.43 (d, J=7.9 Hz, 1H), 4.83 (d, J=61.4 Hz, 1H), 4.62 (s, 2H), 4.52-4.30 (m, 1H), 3.90 (s, 1H), 3.83 (t, J=6.8 Hz, 2H), 3.72-3.62 (m, 1H), 3.46 (s, 2H), 3.35-3.26 (m, 2H), 2.87 (t, J=6.8 Hz, 2H). LCMS (ESI) calcd, for C₂₇H₂₅N₈O₄⁺ [M+H]⁺: 525.20, found, 525.2.

### Example 390: Preparation of 5-((4-(1-(benzo[4,5]imidazo[1,2-a]pyrimidin-2-yl)piperidin-4-yl)piperazin-1-yl)methyl)-2-(2,4-dioxotetrahydropyrimidin-1(2H)-yl)isoindoline-1,3-dione (GT-08568)

With reference to the method of Scheme T-3, the target compound (GT-08568) was obtained (white solid, 30 mg, yield 55%). ¹H NMR (400 MHz, DMSO-d₆) δ 10.92 (s, 1H), 9.35 (d, J=7.9 Hz, 1H), 8.25 (s, 2H), 8.06 (d, J=7.7 Hz, 1H), 7.63 (s, 1H), 7.60-7.54 (m, 1H), 7.52-7.48 (m, 1H), 7.46 (dd, J=6.8, 4.5 Hz, 1H), 5.02 (d, J=12.5 Hz, 1H), 4.61 (d, J=12.2 Hz, 1H), 4.41 (s, 2H), 3.82 (t, J=6.8 Hz, 3H), 3.65-3.57 (m, 5H), 3.36-3.30 (m, 3H), 3.17-3.07 (m, 2H), 2.87 (d, J=6.8 Hz, 2H), 2.34 (s, 2H), 1.81 (s, 2H). LCMS (ESI) calcd. for C₃₂H₃₄N₉O₄⁺ [M+H]⁺: 608.27, found, 608.2.

### Example 391: Preparation of 5-(((1-(4-(4-acetyl-1,4-diazepan-1-yl)-6-((2-(thiophen-2-yl)ethyl)amino)-1,3,5-triazin-2-yl)piperidin-4-yl)(methyl)amino)methyl)-2-(2,4-dioxotetrahydropyrimidin-1(2H)-yl)isoindoline-1,3-dione (GT-08569)

With reference to the method of Scheme T-3, the target compound (GT-08569) was obtained (white solid, 24 mg, yield 37%). ¹H NMR (400 MHz, DMSO-d₆) δ 10.93 (s, 1H), 8.41 (d, J=9.2 Hz, 1H), 8.28 (d, J=5.6 Hz, 1H), 8.09 (d, J=7.7 Hz, 1H), 7.37 (s, 1H), 7.00-6.93 (m, 2H), 4.80 (s, 1H), 4.68 (s, 2H), 4.46 (d, J=6.6 Hz, 1H), 3.93 (s, 1H), 3.83 (t, J=6.7 Hz, 3H), 3.79 (s, 3H), 3.62 (d, J=4.7 Hz, 8H), 3.09 (s, 2H), 2.99 (s, 2H), 2.86 (t, J=6.8 Hz, 2H), 2.59 (s, 3H), 2.32 (s, 2H), 2.01 (s, 2H), 1.98 (s, 1H), 1.84 (s, 2H), 1.74 (s, 2H). LCMS (ESI) calcd, for C₃₅H₄₄N₁₁O₅S⁺ [M+H]⁺: 730.32, found, 730.2.

### Example 392: Preparation of 5-(4-((4-(benzo[4,5]imidazo[1,2-a]pyrimidin-2-yl)piperazin-1-yl)methyl)piperidin-1-yl)-2-(2,4-dioxotetrahydropyrimidin-1(2H)-yl)isoindoline-1,3-dione (GT-08570)

With reference to the method of Scheme T-3, the target compound (GT-08570) was obtained (white solid, 24 mg, yield 46%). ¹H NMR (400 MHz, DMSO-d₆) δ 10.85 (s, 1H), 9.47 (d, J=7.9 Hz, 1H), 8.30 (d, J=8.1 Hz, 1H), 7.72 (d, J=8.6 Hz, 1H), 7.69 (d, J=8.1 Hz, 1H), 7.60 (s, 1H), 7.53 (d, J=8.2 Hz, 1H), 7.48 (d, J=7.9 Hz, 1H), 7.41 (d, J=2.0 Hz, 1H), 7.35-7.30 (m, 1H), 4.13 (d, J=13.1 Hz, 2H), 3.79 (t, J=6.8 Hz, 3H), 3.07 (d, J=13.5 Hz, 3H), 2.83 (t, J=7.3 Hz, 2H), 2.26 (s, 1H), 2.04 (d, J=11.3 Hz, 2H), 1.31 (d, J=11.8 Hz, 2H). LCMS (ESI) calcd, for C₃₂H₃₄N₉O₄⁺ [M+H]⁺: 608.27, found, 608.2.

### Example 393: Preparation of 4-(4-((4-(benzo[4,5]imidazo[1,2-a]pyrimidin-2-yl)piperazin-1-yl)methyl)piperidin-1-yl)-2-(2,4-dioxotetrahydropyrimidin-1(2H)-yl)isoindoline-1,3-dione (GT-08571)

With reference to the method of Scheme T-3, the target compound (GT-08571) was obtained (white solid, 29 mg, yield 56%). ¹H NMR (400 MHz, DMSO-d₆) δ 10.86 (s, 1H), 9.47 (d, J=7.9 Hz, 1H), 8.29 (d, J=8.0 Hz, 1H), 7.76 (d, J=1.0 Hz, 1H), 7.68 (s, 1H), 7.60 (s, 1H), 7.53 (s, 1H), 7.48 (d, J=7.9 Hz, 1H), 7.43 (d, J=8.5 Hz, 1H), 7.40 (d, J=7.1 Hz, 1H), 3.78 (d, J=6.6 Hz, 2H), 3.70 (d, J=12.0 Hz, 2H), 3.15 (s, 2H), 2.95 (dd, J=18.1, 9.7 Hz, 2H), 2.84 (d, J=1.6 Hz, 2H), 2.11 (d, J=11.8 Hz, 3H), 1.55-1.40 (m, 2H). LCMS (ESI) calcd, for C₃₂H₃₄N₉O₄⁺ [M+H]⁺: 608.27, found, 608.2.

### Example 394: Preparation of 5-(4-((1-(benzo[4,5]imidazo[1,2-a]pyrimidin-2-yl)piperidin-4-yl)methyl)piperazin-1-yl)-2-(2,4-dioxotetrahydropyrimidin-1(2H)-yl)isoindoline-1,3-dione (GT-08597)

With reference to the method of Scheme T-3, the target compound (GT-08597) was obtained (white solid, 58 mg, yield 84%). ¹H NMR (400 MHz, DMSO-d₆) δ 10.87 (s, 1H), 9.30 (d, J=7.9 Hz, 1H), 8.24 (d, J=8.1 Hz, 1H), 7.82 (d, J=8.5 Hz, 1H), 7.63 (d, J=7.9 Hz, 1H), 7.55 (dd, J=11.4, 4.0 Hz, 2H), 7.52-7.46 (m, 1H), 7.42 (dd, J=11.0, 4.9 Hz, 2H), 4.87 (d, J=12.2 Hz, 1H), 4.44 (d, J=12.7 Hz, 1H), 4.22 (s, 2H), 3.80 (t, J=6.8 Hz, 2H), 3.66 (d, J=25.4 Hz, 4H), 3.41 (s, 2H), 3.18 (d, J=11.7 Hz, 2H), 3.09 (d, J=6.0 Hz, 2H), 2.84 (t, J=7.0 Hz, 2H), 2.38 (s, 1H), 2.23-2.08 (m, 2H), 1.32 (d, J=11.4 Hz, 2H). LCMS (ESI) calcd. for C₃₂H₃₄N₉O₄⁺ [M+H]⁺: 608.27, found, 608.2.

### Example 395: Preparation of 5-((1S,4S)-5-((1-(benzo[4,5]imidazo[1,2-a]pyrimidin-2-yl)piperidin-4-yl)methyl)-2,5-diazabicyclo[2.2.1]heptan-2-yl)-2-(2,4-dioxotetrahydropyrimidin-1(2H)-yl)isoindoline-1,3-dione (GT-08605)

With reference to the method of Scheme T-3, the target compound (GT-08605) was obtained (white solid, 59 mg, yield 84%). ¹H NMR (400 MHz, DMSO-d₆) δ 10.85 (s, 1H), 9.30 (d, J=7.8 Hz, 1H), 8.23 (d, J=8.1 Hz, 1H), 7.75 (d, J=8.0 Hz, 1H), 7.63 (d, J=7.9 Hz, 1H), 7.55 (t, J=7.6 Hz, 1H), 7.47 (t, J=7.7 Hz, 1H), 7.41 (d, J=7.9 Hz, 1H), 7.19 (s, 1H), 7.01 (s, 1H), 5.02 (s, 1H), 4.85 (d, J=12.6 Hz, 1H), 4.67 (d, J=42.1 Hz, 1H), 4.42 (d, J=12.2 Hz, 1H), 3.79 (t, J=6.7 Hz, 2H), 3.72 (d, J=10.2 Hz, 2H), 3.41 (s, 2H), 3.32 (s, 2H), 3.14 (s, 2H), 3.05 (s, 1H), 2.84 (dd, J=7.9, 6.6 Hz, 2H), 2.27 (s, 1H), 2.22 (s, 1H), 2.15 (d, J=25.7 Hz, 2H), 1.31 (d, J=10.7 Hz, 2H). LCMS (ESI) calcd, for C₃₃H₃₄N₉O₄⁺ [M+H]⁺: 620.27, found, 620.2.

### Example 396: Preparation of 5-((1R,4R)-5-((1-(benzo[4,5]imidazo[1,2-a]pyrimidin-2-yl)piperidin-4-yl)methyl)-2,5-diazabicyclo[2.2.1]heptan-2-yl)-2-(2,4-dioxotetrahydropyrimidin-1(2H)-yl)isoindoline-1,3-dione (GT-08606)

With reference to the method of Scheme T-3, the target compound (GT-08606) was obtained (white solid, 46 mg, yield 66%). ¹H NMR (400 MHz, DMSO-d₆) δ 10.85 (s, 1H), 9.28 (d, J=7.9 Hz, 1H), 8.22 (d, J=8.1 Hz, 1H), 7.76 (d, J=8.4 Hz, 1H), 7.62 (d, J=7.9 Hz, 1H), 7.56 (d, J=7.3 Hz, 1H), 7.48 (d, J=7.4 Hz, 1H), 7.40 (d, J=7.9 Hz, 1H), 7.19 (s, 1H), 7.02 (s, 1H), 5.08-4.76 (m, 2H), 4.66 (d, J=40.9 Hz, 1H), 4.42 (d, J=10.1 Hz, 1H), 4.23 (s, 1H), 3.80 (d, J=6.8 Hz, 2H), 3.77 (s, 2H), 3.41 (s, 1H), 3.33-3.27 (m, 2H), 3.13 (s, 2H), 3.08-2.92 (m, 1H), 2.84 (t, J=7.3 Hz, 2H), 2.23 (t, J=39.7 Hz, 3H), 1.31 (d, J=10.1 Hz, 2H). LCMS (ESI) calcd, for C₃₃H₃₄N₉O₄⁺ [M+H]⁺: 620.27, found, 620.2.

### Example 397: Preparation of 5-(4-((1-(benzo[4,5]imidazo[1,2-a]pyrimidin-2-yl)piperidin-4-yl)methyl)piperazin-1-yl)-2-(2,4-dioxotetrahydropyrimidin-1(2H)-yl)-6-fluoroisoindoline-1,3-dione (GT-08607)

With reference to the method of Scheme T-3, the target compound (GT-08607) was obtained (white solid, 46 mg, yield 65%). ¹H NMR (400 MHz, DMSO-d₆) δ 10.90 (s, 1H), 9.31 (d, J=7.9 Hz, 1H), 8.24 (d, J=8.1 Hz, 1H), 7.90 (d, J=11.4 Hz, 1H), 7.69-7.61 (m, 2H), 7.56 (t, J=7.7 Hz, 1H), 7.49 (d, J=7.4 Hz, 1H), 7.43 (d, J=8.0 Hz, 1H), 4.87 (d, J=11.4 Hz, 1H), 4.44 (d, J=12.7 Hz, 1H), 3.81 (t, J=6.8 Hz, 3H), 3.68 (s, 3H), 3.67-3.57 (m, 2H), 3.30 (s, 2H), 3.23-3.15 (m, 2H), 3.11 (d, J=5.4 Hz, 2H), 2.85 (t, J=6.8 Hz, 2H), 2.37 (s, 1H), 2.21-2.08 (m, 2H), 1.34 (d, J=11.3 Hz, 2H). LCMS (ESI) calcd, for C₃₂H₃₃FN₉O₄⁺[M+H]⁺: 626.26, found, 626.2.

### Example 398: Preparation of 5-(3-((1-(benzo[4,5]imidazo[1,2-a]pyrimidin-2-yl)piperidin-4-yl)methyl)-3,8-diazabicyclo[3.2.1]octan-8-yl)-2-(2,4-dioxotetrahydropyrimidin-1(2H)-yl)isoindoline-1,3-dione (GT-08608)

With reference to the method of Scheme T-3, the target compound (GT-08608) was obtained (white solid, 59 mg, yield 82%). ¹H NMR (400 MHz, DMSO-d₆) δ 10.86 (s, 2H), 9.27 (d, J=7.9 Hz, 1H), 8.22 (d, J=8.1 Hz, 1H), 7.82 (d, J=8.4 Hz, 1H), 7.61 (d, J=7.9 Hz, 1H), 7.55 (s, 1H), 7.48 (d, J=7.5 Hz, 1H), 7.46 (s, 1H), 7.38 (d, J=8.0 Hz, 1H), 7.33 (d, J=8.4 Hz, 1H), 4.80 (s, 3H), 4.35 (d, J=12.8 Hz, 1H), 3.80 (t, J=6.8 Hz, 2H), 3.42 (s, 3H), 3.33 (s, 2H), 3.16 (d, J=12.1 Hz, 1H), 3.13-3.00 (m, 2H), 2.92 (s, 2H), 2.84 (dd, J=8.1, 6.6 Hz, 2H), 2.33 (s, 1H), 2.09 (s, 2H), 2.00 (d, J=11.7 Hz, 2H), 1.29 (d, J=10.7 Hz, 2H). LCMS (ESI) calcd. for C₃₄H₃₆N₉O₄*[M+H]⁺: 634.29, found, 634.2.

### Example 399: Preparation of 5-(6-((1-(benzo[4,5]imidazo[1,2-a]pyrimidin-2-yl)piperidin-4-yl)methyl)-3,6-diazabicyclo[3.1.1]heptan-3-yl)-2-(2,4-dioxotetrahydropyrimidin-1(2H)-yl)isoindoline-1,3-dione (GT-08609)

With reference to the method of Scheme T-3, the target compound (GT-08609) was obtained (white solid, 48 mg, yield 68%). ¹H NMR (400 MHz, DMSO-d₆) δ 10.85 (s, 1H), 9.28 (d, J=7.9 Hz, 1H), 8.22 (d, J=7.9 Hz, 1H), 7.84 (d, J=8.5 Hz, 1H), 7.62 (d, J=7.8 Hz, 1H), 7.56 (d, J=7.4 Hz, 1H), 7.48 (d, J=7.7 Hz, 1H), 7.40 (d, J=7.5 Hz, 1H), 7.23 (s, 1H), 7.16 (d, J=8.7 Hz, 1H), 4.87 (s, 1H), 4.66 (s, 2H), 4.43 (s, 1H), 4.34-4.14 (m, 1H), 3.99 (dd, J=27.8, 11.8 Hz, 3H), 3.80 (d, J=5.0 Hz, 2H), 3.48 (s, 1H), 3.15 (d, J=31.1 Hz, 2H), 2.84 (t, J=6.6 Hz, 3H), 2.32 (d, J=13.7 Hz, 1H), 2.04 (s, 2H), 1.97 (s, 1H), 1.87 (d, J=12.8 Hz, 1H), 1.32 (s, 2H). LCMS (ESI) calcd, for C₃₄H₃₄N₉O₄⁺ [M+H]⁺: 620.27, found, 620.2.

### Example 400: Preparation of 5-(3-((1-(benzo[4,5]imidazo[1,2-a]pyrimidin-2-yl)piperidin-4-yl)methyl)-3,6-diazabicyclo[3.1.1]heptan-6-yl)-2-(2,4-dioxotetrahydropyrimidin-1(2H)-yl)isoindoline-1,3-dione (GT-08610)

With reference to the method of Scheme T-3, the target compound (GT-08610) was obtained (white solid, 9 mg, yield 13%). ¹H NMR (400 MHz, DMSO-d₆) δ 10.86 (s, 1H), 9.25 (d, J=7.9 Hz, 1H), 8.20 (d, J=8.1 Hz, 2H), 7.84 (d, J=8.1 Hz, 1H), 7.61 (d, J=8.1 Hz, 2H), 7.55 (t, J=7.2 Hz, 2H), 7.47 (dd, J=11.2, 4.1 Hz, 2H), 7.37 (d, J=7.8 Hz, 1H), 7.23 (s, 1H), 7.01 (d, J=8.2 Hz, 1H), 4.68 (s, 2H), 4.32 (s, 2H), 3.79 (t, J=6.8 Hz, 4H), 3.10 (d, J=10.4 Hz, 2H), 2.84 (dd, J=7.7, 5.6 Hz, 5H), 2.24 (s, 1H), 2.04 (s, 3H), 1.83 (d, J=39.6 Hz, 1H), 1.23 (s, 3H). LCMS (ESI) calcd, for C₃₄H₃₄N₉O₄⁺ [M+H]⁺: 620.27, found, 620.2.

### Example 401: Preparation of 5-(8-((1-(benzo[4,5]imidazo[1,2-a]pyrimidin-2-yl)piperidin-4-yl)methyl)-3,8-diazabicyclo[3.2.1]octan-3-yl)-2-(2,4-dioxotetrahydropyrimidin-1(2H)-yl)isoindoline-1,3-dione (GT-08822)

With reference to the method of Scheme T-3, the target compound (GT-08822) was obtained (white solid, 54 mg, yield 75%). ¹H NMR (400 MHz, DMSO-d₆) δ 10.87 (s, 1H), 9.31 (d, J=7.9 Hz, 1H), 8.24 (d, J=8.1 Hz, 1H), 7.81 (d, J=8.6 Hz, 1H), 7.63 (d, J=7.9 Hz, 1H), 7.56 (s, 1H), 7.49 (d, J=7.3 Hz, 1H), 7.44 (t, J=7.7 Hz, 2H), 7.33-7.26 (m, 1H), 4.88 (d, J=11.7 Hz, 1H), 4.45 (d, J=12.1 Hz, 1H), 4.23 (s, 2H), 4.00 (s, 2H), 3.95 (s, 2H), 3.80 (t, J=6.9 Hz, 2H), 3.41 (s, 1H), 3.19 (t, J=12.0 Hz, 1H), 3.00 (s, 2H), 2.84 (t, J=7.0 Hz, 2H), 2.44 (s, 1H), 2.22 (s, 4H), 1.97 (d, J=8.5 Hz, 2H), 1.38 (d, J=10.7 Hz, 2H). LCMS (ESI) calcd. for C₃₄H₃₆N₉O₄⁺ [M+H]⁺: 634.29, found, 634.2.

### Example 402: Preparation of 4-(4-((1-(benzo[4,5]imidazo[1,2-a]pyrimidin-2-yl)piperidin-4-yl)methyl)piperazin-1-yl)-2-(2,4-dioxotetrahydropyrimidin-1(2H)-yl)isoindoline-1,3-dione (GT-08823)

With reference to the method of Scheme T-3, the target compound (GT-08823) was obtained (white solid, 42 mg, yield 61%). ¹H NMR (400 MHz, DMSO-d₆) δ 10.89 (s, 1H), 9.29 (d, J=7.9 Hz, 1H), 8.23 (d, J=8.1 Hz, 1H), 7.88-7.78 (m, 1H), 7.62 (d, J=7.8 Hz, 1H), 7.57 (d, J=7.3 Hz, 1H), 7.51 (d, J=7.2 Hz, 2H), 7.49 (s, 1H), 7.43 (d, J=8.0 Hz, 1H), 4.87 (d, J=11.9 Hz, 1H), 4.44 (d, J=12.7 Hz, 1H), 3.87 (s, 1H), 3.80 (t, J=6.8 Hz, 3H), 3.69 (s, 4H), 3.25 (s, 2H), 3.20 (d, J=12.7 Hz, 2H), 3.13 (s, 2H), 2.84 (dt, J=11.2, 6.8 Hz, 2H), 2.36 (d, J=22.4 Hz, 1H), 2.15 (s, 2H), 1.34 (d, J=11.5 Hz, 2H). LCMS (ESI) calcd, for C₃₂H₃₄N₉O₄⁺ [M+H]⁺: 608.27, found, 608.2.

### Example 403: Preparation of 5-(4-((1-(benzo[4,5]imidazo[1,2-a]pyrimidin-2-yl)piperidin-4-yl)methyl)piperazin-1-yl)-2-(2,4-dioxotetrahydropyrimidin-1(2H)-yl)-4-fluoroisoindoline-1,3-dione (GT-08824)

With reference to the method of Scheme T-3, the target compound (GT-08824) was obtained (white solid, 39 mg, yield 55%). ¹H NMR (400 MHz, DMSO-d₆) δ 10.91 (s, 1H), 9.31 (d, J=7.9 Hz, 1H), 8.24 (d, J=8.2 Hz, 1H), 7.76 (d, J=8.1 Hz, 1H), 7.63 (d, J=7.9 Hz, 1H), 7.55 (t, J=7.7 Hz, 2H), 7.49 (d, J=7.5 Hz, 1H), 7.43 (d, J=8.0 Hz, 1H), 4.87 (d, J=12.0 Hz, 1H), 4.44 (d, J=12.7 Hz, 1H), 3.80 (t, J=6.8 Hz, 2H), 3.74 (s, 4H), 3.61 (s, 2H), 3.31 (d, J=6.0 Hz, 2H), 3.24-3.15 (m, 2H), 3.12 (d, J=5.6 Hz, 2H), 2.84 (t, J=6.8 Hz, 2H), 2.38 (s, 1H), 2.23-2.08 (m, 2H), 1.34 (d, J=11.0 Hz, 2H). LCMS (ESI) calcd, for C₃₂H₃₃FN₉O₄⁺ [M+H]⁺: 626.26, found, 626.2.

### Example 404: Preparation of 6-(4-((1-(benzo[4,5]imidazo[1,2-a]pyrimidin-2-yl)piperidin-4-yl)methyl)piperazin-1-yl)-2-(2,4-dioxotetrahydropyrimidin-1(2H)-yl)-4-fluoroisoindoline-1,3-dione (GT-08825)

With reference to the method of Scheme T-3, the target compound (GT-08825) was obtained (white solid, 43 mg, yield 61%). ¹H NMR (400 MHz, DMSO-d₆) δ 10.89 (s, 1H), 9.29 (d, J=7.8 Hz, 1H), 8.23 (d, J=8.0 Hz, 1H), 7.63 (d, J=8.0 Hz, 1H), 7.56 (t, J=7.7 Hz, 1H), 7.49 (d, J=8.0 Hz, 1H), 7.46-7.40 (m, 2H), 7.26 (d, J=12.9 Hz, 1H), 4.86 (d, J=10.9 Hz, 1H), 4.43 (d, J=12.9 Hz, 1H), 4.27 (s, 2H), 3.79 (t, J=7.2 Hz, 3H), 3.61 (s, 2H), 3.41 (s, 1H), 3.32-3.28 (m, 1H), 3.17 (d, J=11.5 Hz, 3H), 3.07 (s, 2H), 2.84 (t, J=7.3 Hz, 2H), 2.36 (s, 1H), 2.14 (dd, J=22.4, 13.0 Hz, 2H), 1.31 (d, J=10.7 Hz, 2H). LCMS (ESI) calcd, for C₃₂H₃₃FN₉O₄⁺ [M+H]⁺: 626.26, found, 626.2.

### Example 405: Preparation of 5-(4-(4-((1-(benzo[4,5]imidazo[1,2-a]pyrimidin-2-yl)piperidin-4-yl)methyl)piperazin-1-yl)piperidin-1-yl)-2-(2,4-dioxotetrahydropyrimidin-1(2H)-yl)isoindoline-1,3-dione (GT-08826)

With reference to the method of Scheme T-3, the target compound (GT-08826) was obtained (white solid, 28 mg, yield 36%). ¹H NMR (400 MHz, DMSO-d₆) δ 10.85 (s, 1H), 9.27 (d, J=7.9 Hz, 1H), 8.22 (d, J=8.0 Hz, 1H), 7.75 (d, J=8.6 Hz, 1H), 7.62 (d, J=7.8 Hz, 1H), 7.56 (t, J=7.7 Hz, 1H), 7.48 (t, J=7.2 Hz, 2H), 7.41 (d, J=8.0 Hz, 1H), 7.37 (d, J=8.7 Hz, 1H), 4.85 (d, J=12.2 Hz, 1H), 4.42 (d, J=12.7 Hz, 1H), 4.29 (d, J=11.7 Hz, 2H), 3.84 (s, 2H), 3.79 (t, J=6.8 Hz, 3H), 3.71 (s, 3H), 3.50 (s, 3H), 3.16 (s, 2H), 3.01 (t, J=12.5 Hz, 3H), 2.83 (t, J=7.0 Hz, 2H), 2.29 (d, J=29.4 Hz, 1H), 2.20 (s, 2H), 2.13 (s, 2H), 1.77 (s, 2H), 1.29 (s, 2H). LCMS (ESI) calcd, for C₃₇H₄₃N₁₀O₄⁺ [M+H]⁺: 691.35, found, 691.2.

### Example 406: Preparation of 5-(4-(4-((1-(benzo[4,5]imidazo[1,2-a]pyrimidin-2-yl)piperidin-4-yl)methyl)piperazin-1-yl)-3,3-difluoropiperidin-1-yl)-2-(2,4-dioxotetrahydropyrimidin-1(2H)-yl)isoindoline-1,3-dione (GT-08827)

With reference to the method of Scheme T-3, the target compound (GT-08827) was obtained (white solid, 18 mg, yield 22%). ¹H NMR (400 MHz, DMSO-d₆) δ 10.86 (s, 1H), 9.30 (d, J=7.9 Hz, 1H), 8.23 (d, J=8.1 Hz, 1H), 7.74 (d, J=8.6 Hz, 1H), 7.62 (s, 1H), 7.55 (s, 1H), 7.52 (s, 1H), 7.48 (s, 1H), 7.42 (d, J=8.1 Hz, 2H), 4.84 (d, J=12.7 Hz, 1H), 4.54-4.35 (m, 2H), 4.26 (d, J=12.8 Hz, 1H), 3.79 (t, J=6.8 Hz, 2H), 3.46 (s, 2H), 3.38 (s, 1H), 3.35 (d, J=6.0 Hz, 2H), 3.28-3.20 (m, 2H), 3.17 (d, J=6.3 Hz, 2H), 3.05 (s, 2H), 3.02 (s, 5H), 2.83 (t, J=7.0 Hz, 2H), 2.30 (s, 1H), 2.16-2.02 (m, 2H), 1.88 (d, J=21.5 Hz, 2H), 1.30 (d, J=11.6 Hz, 2H). LCMS (ESI) calcd, for C₃₇H₄₁F₂N₁₀O₄⁺ [M+H]⁺: 727.33, found, 727.2.

### Example 407: Preparation of 5-(5-((1-(benzo[4,5]imidazo[1,2-a]pyrimidin-2-yl)piperidin-4-yl)methyl)-2,5-diazabicyclo[2.2.2]octan-2-yl)-2-(2,4-dioxotetrahydropyrimidin-1(2H)-yl)isoindoline-1,3-dione (GT-08828)

With reference to the method of Scheme T-3, the target compound (GT-08828) was obtained (white solid, 52 mg, yield 73%). ¹H NMR (400 MHz, DMSO-d₆) δ 10.85 (s, 1H), 9.29 (d, J=7.9 Hz, 1H), 8.23 (d, J=8.1 Hz, 1H), 7.78 (d, J=8.1 Hz, 1H), 7.62 (d, J=7.9 Hz, 1H), 7.55 (t, J=7.8 Hz, 1H), 7.48 (t, J=7.7 Hz, 1H), 7.42 (d, J=8.1 Hz, 1H), 7.24 (s, 1H), 7.07 (d, J=8.6 Hz, 1H), 4.86 (d, J=11.8 Hz, 1H), 4.62 (s, 1H), 4.43 (d, J=11.1 Hz, 1H), 4.02 (s, 1H), 3.87 (s, 1H), 3.79 (t, J=7.0 Hz, 2H), 3.58 (s, 1H), 3.39 (s, 2H), 3.25 (s, 2H), 3.17 (s, 2H), 2.84 (t, J=7.2 Hz, 2H), 2.62 (d, J=41.6 Hz, 1H), 2.33 (s, 1H), 2.17 (s, 2H), 1.98 (s, 2H), 1.86 (d, J=13.8 Hz, 1H), 1.33 (s, 2H). LCMS (ESI) calcd, for C₃₄H₃₆N₉O₄⁺ [M+H]⁺: 634.29, found, 634.2.

### Example 408: Preparation of(S)-5-chloro-4-(4-chloro-1-methyl-1H-pyrazol-5-yl)-N-(1-(((2-(2,4-dioxotetrahydropyrimidin-1(2H)-yl)-1-oxoisoindolin-5-yl)methyl)amino)-3-(3-fluorophenyl) propan-2-yl)thiophene-2-carboxamide (GT-08572)

With reference to the method of Scheme T-3, the target compound (GT-08572) was obtained (white solid, 27 mg, yield 42%). ¹H NMR (400 MHz, DMSO-d₆) δ 10.69 (s, 1H), 9.48 (s, 2H), 9.07 (d, J=7.9 Hz, 1H), 8.24 (s, 1H), 8.17 (d, J=1.1 Hz, 1H), 8.12 (s, 1H), 7.81 (d, J=4.7 Hz, 2H), 7.74 (s, 1H), 7.30 (d, J=6.5 Hz, 1H), 7.13 (dd, J=12.2, 6.8 Hz, 3H), 7.01 (s, 1H), 4.56 (s, 2H), 4.37 (d, J=12.9 Hz, 2H), 3.87 (s, 4H), 3.78 (d, J=6.4 Hz, 2H), 2.98 (d, J=7.1 Hz, 2H), 2.90-2.81 (m, 1H), 2.78 (s, 1H). LCMS (ESI) calcd. for C₃₁H₂₉Cl₂FN₇O₄S⁺ [M+H]⁺: 684.14, found, 684.2.

### Example 409: Preparation of 1-(5-(((1-(4-(2-(2-aminopyridin-3-yl)-5-phenyl-3H-imidazo[4,5-b]pyridin-3-yl)phenyl)cyclobutyl)amino)methyl)-1-oxoisoindolin-2-yl)dihydropyrimidine-2,4 (1H,3H)-dione (GT-08573)

With reference to the method of Scheme T-3, the target compound (GT-08573) was obtained (white solid, 40 mg, yield 62%). ¹H NMR (400 MHz, DMSO-d₆) δ 10.69 (s, 1H), 10.51 (s, 2H), 8.39 (d, J=8.4 Hz, 2H), 8.18 (dd, J=6.2, 1.3 Hz, 1H), 8.09 (d, J=8.4 Hz, 3H), 7.97 (d, J=8.5 Hz, 2H), 7.90 (d, J=6.4 Hz, 1H), 7.79 (s, 1H), 7.76 (d, J=8.5 Hz, 2H), 7.69 (s, 1H), 7.57 (d, J=8.1 Hz, 1H), 7.48 (s, 1H), 7.46 (s, 1H), 6.87-6.82 (m, 1H), 4.77 (d, J=16.8 Hz, 1H), 4.52 (d, J=16.4 Hz, 1H), 3.81-3.76 (m, 3H), 2.95 (dd, J=20.4, 8.4 Hz, 2H), 2.88-2.82 (m, 1H), 2.77 (s, 1H), 2.66 (s, 3H), 2.20-2.08 (m, 1H), 1.79 (dd, J=19.4, 8.8 Hz, 1H). LCMS (ESI) calcd, for C₄₀H₃₆N₉O₃⁺ [M+H]⁺: 690.29, found, 690.2.

### Example 410: Preparation of(S)-5-chloro-4-(4-chloro-1-methyl-1H-pyrazol-5-yl)-N-(1-(3,4-difluorophenyl)-3-(((2-(2,4-dioxotetrahydropyrimidin-1(2H)-yl)-1-oxoisoindolin-5-yl)methyl)amino) propan-2-yl) furan-2-carboxamide (GT-08574)

With reference to the method of Scheme T-3, the target compound (GT-08574) was obtained (white solid, 42 mg, yield 65%). ¹H NMR (400 MHz, DMSO-d₆) δ 10.69 (s, 1H), 9.56 (d, J=22.9 Hz, 2H), 8.92 (d, J=8.8 Hz, 1H), 7.84 (d, J=7.4 Hz, 2H), 7.75 (d, J=8.1 Hz, 1H), 7.71 (s, 1H), 7.67 (s, 1H), 7.35 (s, 1H), 7.33 (d, J=2.4 Hz, 1H), 7.11 (s, 1H), 4.80 (d, J=16.6 Hz, 1H), 4.58 (s, 2H), 4.44-4.26 (m, 2H), 3.81 (d, J=7.0 Hz, 2H), 3.77 (s, 3H), 3.16 (s, 2H), 2.97 (s, 2H), 2.89-2.83 (m, 1H), 2.81-2.73 (m, 1H). LCMS (ESI) calcd. for C₃₁H₂₈C₁₂F₂N₇O₅⁺ [M+H]⁺: 686.15, found, 686.2.

### Example 411: Preparation of(S)-5-chloro-4-(4-chloro-1-methyl-1H-pyrazol-5-yl)-N-(1-(3,4-difluorophenyl)-3-(4-((2-(2,4-dioxotetrahydropyrimidin-1(2H)-yl)-1-oxoisoindolin-5-yl)methyl)piperazin-1-yl) propan-2-yl) furan-2-carboxamide (GT-08575)

With reference to the method of Scheme T-3, the target compound (GT-08575) was obtained (white solid, 59 mg, yield 84%). ¹H NMR (400 MHz, DMSO-d₆) δ 10.69 (s, 1H), 8.89 (s, 1H), 7.84 (d, J=7.9 Hz, 1H), 7.78 (s, 1H), 7.71 (s, 1H), 7.62 (s, 1H), 7.43-7.36 (m, 1H), 7.32 (d, J=10.8 Hz, 1H), 7.13 (s, 1H), 4.80 (d, J=16.7 Hz, 1H), 4.56 (d, J=16.3 Hz, 2H), 4.39 (s, 2H), 3.80 (d, J=6.9 Hz, 2H), 3.76 (s, 4H), 3.59 (s, 3H), 3.39-3.26 (m, 4H), 3.12 (dd, J=7.4, 4.2 Hz, 2H), 3.00 (d, J=10.2 Hz, 1H), 2.87 (d, J=8.4 Hz, 2H), 2.77 (dd, J=13.4, 7.6 Hz, 1H). LCMS (ESI) calcd, for C₃₅H₃₅Cl₂F₂N₈O₅⁺ [M+H]⁺: 755.21, found, 755.2.

### Example 412: Preparation of(S)-5-chloro-4-(4-chloro-1-methyl-1H-pyrazol-5-yl)-N-(1-(4-((2-(2,4-dioxotetrahydropyrimidin-1(2H)-yl)-1-oxoisoindolin-5-yl)methyl)piperazin-1-yl)-3-(3-fluorophenyl) propan-2-yl)thiophene-2-carboxamide (GT-08576)

With reference to the method of Scheme T-3, the target compound (GT-08576) was obtained (white solid, 43 mg, yield 61%). ¹H NMR (400 MHz, DMSO-d₆) δ 10.69 (s, 1H), 9.06 (s, 1H), 8.22 (s, 1H), 8.15 (d, J=1.2 Hz, 1H), 7.85 (s, 1H), 7.83 (s, 1H), 7.77 (s, 1H), 7.28 (d, J=6.4 Hz, 1H), 7.16 (t, J=11.0 Hz, 2H), 6.99 (s, 1H), 4.77 (s, 1H), 4.56 (d, J=16.5 Hz, 2H), 4.33 (d, J=42.7 Hz, 2H), 3.87 (s, 3H), 3.79 (t, J=6.8 Hz, 2H), 3.66 (d, J=21.5 Hz, 2H), 3.53 (s, 3H), 3.38-3.30 (m, 3H), 3.26-3.10 (m, 2H), 3.02 (s, 2H), 2.85 (s, 1H), 2.78 (s, 1H). LCMS (ESI) calcd, for C₃₅H₃₆Cl₂FN₈O₄S⁺ [M+H]⁺: 753.19, found, 753.2.

### Example 413: Preparation of(S)-5-chloro-4-(4-chloro-1-methyl-1H-pyrazol-5-yl)-N-(1-(((2-(2,4-dioxotetrahydropyrimidin-1(2H)-yl)-1,3-dioxoisoindolin-5-yl)methyl)amino)-3-(3-fluorophenyl) propan-2-yl)thiophene-2-carboxamide (GT-08577)

With reference to the method of Scheme T-3, the target compound (GT-08577) was obtained (white solid, 11 mg, yield 18%). ¹H NMR (400 MHz, DMSO-d₆) δ 10.92 (s, 1H), 9.49 (s, 2H), 9.01 (s, 1H), 8.20 (d, J=24.5 Hz, 3H), 8.08 (d, J=8.4 Hz, 2H), 7.30 (dd, J=14.4, 7.8 Hz, 1H), 7.11 (d, J=7.6 Hz, 2H), 7.02 (t, J=8.7 Hz, 1H), 4.53 (d, J=5.7 Hz, 1H), 4.43 (s, 2H), 3.86 (s, 3H), 3.83 (t, J=6.7 Hz, 2H), 3.20 (s, 2H), 2.98 (d, J=7.0 Hz, 2H), 2.86 (t, J=6.7 Hz, 2H). LCMS (ESI) calcd, for C₃₁H₂₇C₁₂FN₇O₅S⁺ [M+H]⁺: 698.11, found, 698.2.

### Example 414: Preparation of 5-(((1-(4-(2-(2-aminopyridin-3-yl)-5-phenyl-3H-imidazo[4,5-b]pyridin-3-yl)phenyl)cyclobutyl)amino)methyl)-2-(2,4-dioxotetrahydropyrimidin-1(2H)-yl)isoindoline-1,3-dione (GT-08578)

With reference to the method of Scheme T-3, the target compound (GT-08578) was obtained (white solid, 28 mg, yield 45%). ¹H NMR (400 MHz, DMSO-d₆) δ 10.93 (s, 1H), 10.46 (s, 2H), 8.38 (d, J=8.4 Hz, 1H), 8.14 (s, 2H), 8.08 (d, J=8.2 Hz, 3H), 8.04 (d, J=7.7 Hz, 1H), 7.95 (d, J=8.4 Hz, 3H), 7.82 (d, J=7.2 Hz, 1H), 7.76 (d, J=8.4 Hz, 2H), 7.47 (d, J=7.7 Hz, 2H), 7.42 (s, 1H), 6.80 (s, 1H), 3.95 (s, 2H), 3.84 (t, J=6.7 Hz, 2H), 2.93 (s, 2H), 2.86 (t, J=6.7 Hz, 2H), 2.71 (s, 2H), 2.16 (d, J=9.5 Hz, 1H), 1.81 (dd, J=18.7, 9.7 Hz, 1H). LCMS (ESI) calcd, for C₄₀H₃₄N₉O₄⁺ [M+H]⁺: 704.27, found, 704.2.

### Example 415: Preparation of(S)-5-chloro-4-(4-chloro-1-methyl-1H-pyrazol-5-yl)-N-(1-(3,4-difluorophenyl)-3-(((2-(2,4-dioxotetrahydropyrimidin-1(2H)-yl)-1,3-dioxoisoindolin-5-yl)methyl)amino) propan-2-yl) furan-2-carboxamide (GT-08579)

With reference to the method of Scheme T-3, the target compound (GT-08579) was obtained (white solid, 8 mg, yield 13%). ¹H NMR (400 MHz, DMSO-d₆) δ 10.92 (s, 1H), 9.38 (s, 2H), 8.80 (d, J=8.7 Hz, 1H), 8.23 (d, J=3.7 Hz, 1H), 8.09 (s, 2H), 7.71 (s, 1H), 7.59 (s, 1H), 7.35 (d, J=10.7 Hz, 2H), 7.10 (s, 1H), 4.56 (s, 1H), 4.43 (s, 2H), 3.83 (t, J=6.8 Hz, 2H), 3.77 (d, J=5.1 Hz, 3H), 3.18 (s, 2H), 2.95 (s, 1H), 2.89 (s, 1H), 2.86 (t, J=6.8 Hz, 2H). LCMS (ESI) calcd, for C₃₁H₂₆Cl₂F₂N₇O₆⁺ [M+H]⁺: 700.13, found, 700.2.

### Example 416: Preparation of(S)-5-chloro-4-(4-chloro-1-methyl-1H-pyrazol-5-yl)-N-(1-(3,4-difluorophenyl)-3-(4-((2-(2,4-dioxotetrahydropyrimidin-1(2H)-yl)-1,3-dioxoisoindolin-5-yl)methyl)piperazin-1-yl) propan-2-yl) furan-2-carboxamide (GT-08580)

With reference to the method of Scheme T-3, the target compound (GT-08580) was obtained (white solid, 33 mg, yield 48%). ¹H NMR (400 MHz, DMSO-d₆) δ 10.92 (s, 1H), 8.87 (s, 1H), 8.20 (s, 1H), 8.06 (s, 2H), 7.71 (s, 1H), 7.61 (s, 1H), 7.43-7.35 (m, 1H), 7.32 (d, J=10.8 Hz, 1H), 7.13 (s, 1H), 4.55 (s, 1H), 4.30 (s, 2H), 3.82 (t, J=6.8 Hz, 2H), 3.76 (s, 3H), 3.65 (s, 1H), 3.58 (s, 2H), 3.36-3.23 (m, 4H), 3.09 (d, J=11.5 Hz, 2H), 2.97 (s, 2H), 2.90 (d, J=8.9 Hz, 1H), 2.86 (t, J=6.8 Hz, 2H). LCMS (ESI) calcd, for C₃₅H₃₃C₁₂F₂N₈O₆⁺ [M+H]⁺: 769.19, found, 769.2.

### Example 417: Preparation of(S)-5-chloro-4-(4-chloro-1-methyl-1H-pyrazol-5-yl)-N-(1-(4-((2-(2,4-dioxotetrahydropyrimidin-1(2H)-yl)-1,3-dioxoisoindolin-5-yl)methyl)piperazin-1-yl)-3-(3-fluorophenyl) propan-2-yl)thiophene-2-carboxamide (GT-08581)

With reference to the method of Scheme T-3, the target compound (GT-08581) was obtained (white solid, 25 mg, yield 37%). ¹H NMR (400 MHz, DMSO-d₆) δ 10.91 (s, 1H), 8.98 (s, 1H), 8.16 (t, J=4.3 Hz, 3H), 8.05 (s, 2H), 7.28 (d, J=6.4 Hz, 1H), 7.14 (d, J=8.0 Hz, 2H), 7.00 (s, 1H), 4.53 (s, 1H), 4.12 (d, J=68.3 Hz, 2H), 3.86 (s, 3H), 3.82 (t, J=6.8 Hz, 2H), 3.59 (s, 3H), 3.38-3.18 (m, 5H), 3.16-3.08 (m, 1H), 3.00 (s, 3H), 2.85 (t, J=6.8 Hz, 2H). LCMS (ESI) calcd, for C₃₅H₃₄Cl₂FN₈O₅S⁺ [M+H]⁺: 767.17, found, 767.2.

### Example 418: Preparation of(S)-5-chloro-4-(4-chloro-1-methyl-1H-pyrazol-5-yl)-N-(1-(((1-(2-(2,4-dioxotetrahydropyrimidin-1(2H)-yl)-1,3-dioxoisoindolin-5-yl)piperidin-4-yl)methyl)amino)-3-(3-fluorophenyl) propan-2-yl)thiophene-2-carboxamide (GT-08582)

With reference to the method of Scheme T-3, the target compound (GT-08582) was obtained (white solid, 15 mg, yield 23%). ¹H NMR (400 MHz, DMSO-d₆) δ 10.84 (s, 1H), 8.98 (d, J=8.2 Hz, 1H), 8.76 (d, J=49.5 Hz, 2H), 8.19 (d, J=8.4 Hz, 2H), 7.72 (d, J=8.6 Hz, 1H), 7.40 (s, 1H), 7.31 (t, J=7.3 Hz, 2H), 7.15 (d, J=8.4 Hz, 2H), 7.03 (s, 1H), 4.52 (s, 1H), 4.10 (d, J=13.0 Hz, 2H), 3.86 (s, 3H), 3.78 (t, J=6.7 Hz, 2H), 3.17 (s, 3H), 2.98 (d, J=5.9 Hz, 3H), 2.92 (s, 2H), 2.83 (t, J=6.9 Hz, 2H), 2.04 (s, 1H), 1.85 (s, 2H), 1.25 (s, 2H). LCMS (ESI) calcd, for C₃₆H₃₆Cl₂FN₈O₅S⁺ [M+H]⁺: 781.19, found, 781.2.

### Example 419: Preparation of 5-(4-(((1-(4-(2-(2-aminopyridin-3-yl)-5-phenyl-3H-imidazo[4,5-b]pyridin-3-yl)phenyl)cyclobutyl)amino)methyl)piperidin-1-yl)-2-(2,4-dioxotetrahydropyrimidin-1(2H)-yl)isoindoline-1,3-dione (GT-08583)

With reference to the method of Scheme T-3, the target compound (GT-08583) was obtained (white solid, 19 mg, yield 29%). ¹H NMR (400 MHz, DMSO-d₆) δ 10.84 (s, 1H), 9.88 (s, 2H), 8.39 (s, 1H), 8.36 (s, 1H), 8.12 (d, J=6.1 Hz, 1H), 8.08 (s, 1H), 8.05 (d, J=9.2 Hz, 3H), 7.92 (d, J=8.5 Hz, 2H), 7.69 (t, J=8.7 Hz, 3H), 7.44 (d, J=7.8 Hz, 2H), 7.39 (d, J=7.2 Hz, 1H), 7.34 (s, 1H), 7.23 (dd, J=8.7, 2.1 Hz, 1H), 6.81-6.75 (m, 1H), 4.03 (d, J=11.2 Hz, 2H), 3.77 (t, J=6.8 Hz, 3H), 2.90 (d, J=11.3 Hz, 4H), 2.83 (t, J=7.1 Hz, 3H), 2.66 (s, 3H), 2.38 (s, 2H), 2.14 (d, J=9.1 Hz, 1H), 1.87 (s, 2H), 1.81-1.74 (m, 1H). LCMS (ESI) calcd. for C₄₅H₄₃N₁₀O₄⁺[M+H]⁺: 787.35, found, 787.2.

### Example 420: Preparation of(S)-5-chloro-4-(4-chloro-1-methyl-1H-pyrazol-5-yl)-N-(1-(3,4-difluorophenyl)-3-(((1-(2-(2,4-dioxotetrahydropyrimidin-1(2H)-yl)-1,3-dioxoisoindolin-5-yl)piperidin-4-yl)methyl)amino) propan-2-yl) furan-2-carboxamide (GT-08584)

With reference to the method of Scheme T-3, the target compound (GT-08584) was obtained (white solid, 37 mg, yield 56%). ¹H NMR (400 MHz, DMSO-d₆) δ 10.84 (s, 1H), 8.87 (d, J=8.8 Hz, 2H), 8.75 (s, 1H), 7.72 (t, J=4.3 Hz, 2H), 7.66 (s, 1H), 7.41 (d, J=2.0 Hz, 1H), 7.37 (s, 1H), 7.33 (dd, J=13.7, 5.2 Hz, 2H), 7.13 (s, 1H), 4.59-4.48 (m, 1H), 4.11 (d, J=13.1 Hz, 2H), 3.80 (s, 1H), 3.77 (s, 3H), 3.76 (s, 1H), 3.17 (d, J=14.7 Hz, 2H), 2.99 (s, 2H), 2.96 (d, J=4.9 Hz, 2H), 2.93-2.88 (m, 2H), 2.83 (t, J=7.2 Hz, 2H), 2.06 (s, 1H), 1.87 (d, J=11.2 Hz, 2H), 1.27 (d, J=7.2 Hz, 2H). LCMS (ESI) calcd, for C₃₆H₃₅Cl₂F₂N₈O₆⁺ [M+H]⁺: 783.20, found, 783.2.

### Example 421: Preparation of(S)-5-chloro-4-(4-chloro-1-methyl-1H-pyrazol-5-yl)-N-(1-(3,4-difluorophenyl)-3-(4-((1-(2-(2,4-dioxotetrahydropyrimidin-1(2H)-yl)-1,3-dioxoisoindolin-5-yl)piperidin-4-yl)methyl)piperazin-1-yl) propan-2-yl) furan-2-carboxamide (GT-08585)

With reference to the method of Scheme T-3, the target compound (GT-08585) was obtained (white solid, 51 mg, yield 71%). ¹H NMR (400 MHz, DMSO-d₆) δ 10.84 (s, 1H), 8.91 (s, 1H), 7.72 (d, J=7.2 Hz, 2H), 7.62 (s, 1H), 7.41 (s, 2H), 7.31 (d, J=8.5 Hz, 2H), 7.15 (s, 1H), 4.58 (s, 1H), 4.12 (d, J=12.9 Hz, 2H), 3.90 (d, J=38.1 Hz, 1H), 3.79 (d, J=6.8 Hz, 2H), 3.77 (s, 3H), 3.57 (s, 4H), 3.54 (s, 2H), 3.39-3.24 (m, 3H), 3.11 (s, 1H), 3.02 (d, J=12.5 Hz, 4H), 2.92 (d, J=10.2 Hz, 1H), 2.83 (t, J=7.2 Hz, 2H), 2.17 (s, 1H), 1.97 (d, J=9.2 Hz, 2H), 1.26 (d, J=10.3 Hz, 2H). LCMS (ESI) calcd, for C₄₀H₄₂Cl₂F₂N₉O₆⁺ [M+H]⁺: 852.26, found, 852.2.

### Example 422: Preparation of(S)-5-chloro-4-(4-chloro-1-methyl-1H-pyrazol-5-yl)-N-(1-(4-((1-(2-(2,4-dioxotetrahydropyrimidin-1(2H)-yl)-1,3-dioxoisoindolin-5-yl)piperidin-4-yl)methyl)piperazin-1-yl)-3-(3-fluorophenyl) propan-2-yl)thiophene-2-carboxamide (GT-08586)

With reference to the method of Scheme T-3, the target compound (GT-08586) was obtained (white solid, 47 mg, yield 65%). ¹H NMR (400 MHz, DMSO-d₆) δ 10.84 (s, 1H), 9.06 (s, 1H), 8.18 (d, J=20.2 Hz, 2H), 7.72 (d, J=8.6 Hz, 1H), 7.41 (s, 1H), 7.31 (dd, J=13.2, 4.7 Hz, 2H), 7.17 (d, J=7.8 Hz, 2H), 7.00 (s, 1H), 4.56 (s, 1H), 4.12 (d, J=12.6 Hz, 2H), 3.87 (s, 3H), 3.78 (t, J=6.8 Hz, 3H), 3.73-3.61 (m, 3H), 3.54 (s, 2H), 3.44-3.38 (m, 2H), 3.35-3.20 (m, 2H), 3.04 (s, 3H), 2.99 (d, J=11.5 Hz, 3H), 2.83 (t, J=7.0 Hz, 2H), 2.17 (s, 1H), 1.95 (s, 2H), 1.26 (d, J=11.9 Hz, 2H). LCMS (ESI) calcd, for C₄₀H₄₃Cl₂FN₉O₅S⁺ [M+H]⁺: 850.25, found, 850.2.

### Example 423: Preparation of(S)-5-chloro-4-(4-chloro-1-methyl-1H-pyrazol-5-yl)-N-(1-(((1-(2-(2,4-dioxotetrahydropyrimidin-1(2H)-yl)-1,3-dioxoisoindolin-4-yl)piperidin-4-yl)methyl)amino)-3-(3-fluorophenyl) propan-2-yl)thiophene-2-carboxamide (GT-08587)

With reference to the method of Scheme T-3, the target compound (GT-08587) was obtained (white solid, 19 mg, yield 29%). ¹H NMR (400 MHz, DMSO-d₆) δ 10.85 (s, 1H), 9.08 (t, J=9.4 Hz, 1H), 8.96 (s, 1H), 8.81 (s, 1H), 8.27 (s, 1H), 8.17 (d, J=1.3 Hz, 1H), 7.79-7.67 (m, 1H), 7.39 (dd, J=7.8, 3.3 Hz, 2H), 7.35-7.29 (m, 1H), 7.15 (d, J=7.8 Hz, 2H), 7.03 (td, J=8.7, 2.1 Hz, 1H), 4.54 (s, 1H), 3.87 (s, 3H), 3.80-3.75 (m, 2H), 3.68 (dd, J=23.4, 13.0 Hz, 2H), 3.25 (s, 1H), 3.17 (s, 1H), 3.03-2.98 (m, 2H), 2.98-2.90 (m, 2H), 2.88 (d, J=10.9 Hz, 1H), 2.83 (d, J=6.8 Hz, 2H), 1.92 (d, J=9.5 Hz, 2H), 1.53-1.32 (m, 2H). LCMS (ESI) calcd. for C₃₆H₃₆Cl₂FN₈O₅S⁺ [M+H]⁺: 781.19, found, 781.2.

### Example 424: Preparation of 4-(4-(((1-(4-(2-(2-aminopyridin-3-yl)-5-phenyl-3H-imidazo[4,5-b]pyridin-3-yl)phenyl)cyclobutyl)amino)methyl)piperidin-1-yl)-2-(2,4-dioxotetrahydropyrimidin-1(2H)-yl)isoindoline-1,3-dione (GT-08588)

With reference to the method of Scheme T-3, the target compound (GT-08588) was obtained (white solid, 24 mg, yield 36%). ¹H NMR (400 MHz, DMSO-d₆) δ 10.85 (s, 1H), 9.82 (d, J=30.2 Hz, 2H), 8.36 (d, J=8.4 Hz, 1H), 8.09-8.02 (m, 4H), 7.93 (d, J=8.5 Hz, 2H), 7.77-7.69 (m, 4H), 7.45 (t, J=7.6 Hz, 2H), 7.41-7.35 (m, 2H), 7.33 (d, J=8.7 Hz, 1H), 6.80-6.69 (m, 1H), 3.77 (t, J=6.8 Hz, 2H), 3.70 (d, J=10.9 Hz, 2H), 3.61 (d, J=10.6 Hz, 2H), 2.88 (s, 2H), 2.86 (d, J=9.9 Hz, 2H), 2.83 (s, 1H), 2.80 (s, 1H), 2.69 (s, 2H), 2.42 (s, 2H), 2.15 (d, J=9.4 Hz, 1H), 1.90 (s, 2H), 1.79 (dd, J=19.2, 8.8 Hz, 2H). LCMS (ESI) calcd, for C₄₅H₄₃N₁₀O₄⁺ [M+H]⁺: 787.35, found, 787.2.

### Example 425: Preparation of(S)-5-chloro-4-(4-chloro-1-methyl-1H-pyrazol-5-yl)-N-(1-(3,4-difluorophenyl)-3-(((1-(2-(2,4-dioxotetrahydropyrimidin-1(2H)-yl)-1,3-dioxoisoindolin-4-yl)piperidin-4-yl)methyl)amino) propan-2-yl) furan-2-carboxamide (GT-08589)

With reference to the method of Scheme T-3, the target compound (GT-08589) was obtained (white solid, 41 mg, yield 62%). ¹H NMR (400 MHz, DMSO-d₆) δ 10.85 (s, 1H), 8.91 (d, J=27.9 Hz, 2H), 8.76 (s, 1H), 7.74 (s, 1H), 7.71 (s, 1H), 7.66 (s, 1H), 7.43-7.39 (m, 2H), 7.38 (d, J=4.1 Hz, 1H), 7.34 (d, J=10.9 Hz, 1H), 7.14 (s, 1H), 4.56 (s, 1H), 3.80 (d, J=10.3 Hz, 1H), 3.77 (s, 4H), 3.75 (s, 1H), 3.69 (d, J=11.6 Hz, 1H), 3.17 (s, 2H), 2.96 (d, J=6.5 Hz, 3H), 2.93 (s, 3H), 2.83 (d, J=6.7 Hz, 2H), 1.93 (s, 3H), 1.44 (d, J=11.0 Hz, 2H). LCMS (ESI) calcd, for C₃₆H₃₅Cl₂F₂N₈O₆⁺ [M+H]⁺: 783.20, found, 783.2.

### Example 426: Preparation of(S)-5-chloro-4-(4-chloro-1-methyl-1H-pyrazol-5-yl)-N-(1-(3,4-difluorophenyl)-3-(4-((1-(2-(2,4-dioxotetrahydropyrimidin-1(2H)-yl)-1,3-dioxoisoindolin-4-yl)piperidin-4-yl)methyl)piperazin-1-yl) propan-2-yl) furan-2-carboxamide (GT-08590)

With reference to the method of Scheme T-3, the target compound (GT-08590) was obtained (white solid, 41 mg, yield 57%). ¹H NMR (400 MHz, DMSO-d₆) δ 10.85 (s, 1H), 8.84 (s, 1H), 7.73 (d, J=7.4 Hz, 1H), 7.71 (s, 1H), 7.59 (s, 1H), 7.41 (d, J=7.9 Hz, 2H), 7.38 (s, 1H), 7.35-7.29 (m, 1H), 7.14 (s, 1H), 4.55 (s, 1H), 3.90 (d, J=39.1 Hz, 1H), 3.79 (d, J=6.6 Hz, 2H), 3.77 (s, 3H), 3.74 (s, 1H), 3.68 (d, J=11.8 Hz, 2H), 3.46 (s, 4H), 3.30-3.19 (m, 2H), 3.10 (s, 2H), 3.01 (d, J=13.6 Hz, 2H), 2.92 (d, J=17.9 Hz, 2H), 2.90 (s, 1H), 2.86-2.80 (m, 2H), 2.56 (s, 1H), 2.00 (s, 3H), 1.43 (s, 2H). LCMS (ESI) calcd, for C₄₀H₄₂Cl₂F₂N₉O₆⁺ [M+H]⁺: 852.26, found, 852.2.

### Example 427: Preparation of(S)-5-chloro-4-(4-chloro-1-methyl-1H-pyrazol-5-yl)-N-(1-(4-((1-(2-(2,4-dioxotetrahydropyrimidin-1(2H)-yl)-1,3-dioxoisoindolin-4-yl)piperidin-4-yl)methyl)piperazin-1-yl)-3-(3-fluorophenyl) propan-2-yl)thiophene-2-carboxamide (GT-08591)

With reference to the method of Scheme T-3, the target compound (GT-08591) was obtained (white solid, 45 mg, yield 63%). ¹H NMR (400 MHz, DMSO-d₆) δ 10.85 (s, 1H), 8.99 (s, 1H), 8.17 (d, J=12.0 Hz, 2H), 7.74 (t, J=7.7 Hz, 1H), 7.40 (t, J=7.4 Hz, 2H), 7.29 (d, J=7.4 Hz, 1H), 7.17 (t, J=10.6 Hz, 2H), 7.00 (s, 1H), 4.54 (s, 1H), 3.97 (d, J=59.6 Hz, 1H), 3.87 (s, 3H), 3.80 (s, 2H), 3.79-3.75 (m, 2H), 3.74 (s, 1H), 3.69 (s, 2H), 3.48 (s, 3H), 3.37-3.25 (m, 3H), 3.05 (d, J=10.1 Hz, 3H), 2.94 (s, 3H), 2.83 (d, J=4.1 Hz, 2H), 2.01 (s, 3H), 1.45 (d, J=10.2 Hz, 2H). LCMS (ESI) calcd, for C₄₀H₄₃Cl₂FN₉O₅S⁺ [M+H]⁺: 850.25, found, 850.2.

### Example 428: Preparation of 6-(6-(4-((2-(2,4-dioxotetrahydropyrimidin-1(2H)-yl)-1-oxoisoindolin-5-yl)methyl)piperazin-1-yl)pyridin-3-yl)-1-isopropyl-N-((6-methyl-2-oxo-4-propyl-1,2-dihydropyridin-3-yl)methyl)-1H-indazole-4-carboxamide (GT-08418)

With reference to the method of Scheme T-3, the target compound (GT-08418) was obtained (white solid, 26 mg, yield 42%). ¹H NMR (400 MHz, DMSO-d₆) δ 11.80 (s, 1H), 11.64 (s, 1H), 10.70 (s, 1H), 8.67 (d, J=1.9 Hz, 2H), 8.37 (s, 1H), 8.29 (d, J=7.2 Hz, 1H), 8.16 (s, 1H), 7.95 (s, 1H), 7.90-7.82 (m, 3H), 7.22 (d, J=9.1 Hz, 1H), 5.95 (s, 1H), 5.23-5.08 (m, 1H), 4.83 (d, J=16.2 Hz, 1H), 4.64-4.47 (m, 5H), 4.43 (d, J=4.7 Hz, 2H), 3.82-3.78 (m, 4H), 3.57-3.49 (m, 2H), 3.48-3.40 (m, 2H), 3.19 (s, 2H), 2.93-2.83 (m, 1H), 2.81-2.74 (m, 1H), 2.58-2.53 (m, 2H), 2.15 (s, 3H), 1.59-1.52 (m, 2H), 1.50 (d, J=6.5 Hz, 6H). LCMS (ESI) calcd, for C₄₃H₄₉N₁₀O₅⁺ [M+H]⁺: 785.39, found, 785.2.

### Example 429: Preparation of 6-(6-(4-((2-(2,4-dioxotetrahydropyrimidin-1(2H)-yl)-1,3-dioxoisoindolin-5-yl)methyl)piperazin-1-yl)pyridin-3-yl)-1- isopropyl-N-((6-methyl-2-oxo-4-propyl-1,2-dihydropyridin-3-yl)methyl)-1H-indazole-4-carboxamide (GT-08419)

With reference to the method of Scheme T-3, the target compound (GT-08419) was obtained (white solid, 42 mg, yield 67%). ¹H NMR (400 MHz, DMSO-d₆) δ 11.90 (s, 1H), 11.61 (s, 1H), 10.94 (s, 1H), 8.68 (d, J=2.2 Hz, 1H), 8.65 (t, J=4.8 Hz, 1H), 8.36 (d, J=5.8 Hz, 2H), 8.29-8.18 (m, 2H), 8.12 (d, J=8.1 Hz, 2H), 7.84 (s, 1H), 7.18 (d, J=9.1 Hz, 1H), 5.94 (s, 1H), 5.20-5.08 (m, 1H), 4.62 (s, 2H), 4.48 (s, 2H), 4.42 (d, J=4.7 Hz, 2H), 3.83 (t, J=6.8 Hz, 2H), 3.51-3.43 (m, 3H), 3.19 (s, 2H), 2.87 (t, J=6.8 Hz, 2H), 2.57-2.52 (m, 2H), 2.14 (s, 3H), 1.57-1.51 (m, 2H), 1.50 (d, J=6.6 Hz, 6H), 0.88 (t, J=7.3 Hz, 3H). LCMS (ESI) calcd. for C₄₃H₄₇N₁₀O₆⁺ [M+H]⁺: 799.37, found, 799.2.

### Example 430: Preparation of 6-(6-(4-(((2-(2,4-dioxotetrahydropyrimidin-1(2H)-yl)-1-oxoisoindolin-5-yl)methyl)(methyl)amino)piperidin-1-yl)pyridin-3-yl)-1-isopropyl-N-((6-methyl-2-oxo-4-propyl-1,2-dihydropyridin-3-yl)methyl)-1H-indazole-4-carboxamide (GT-08420)

With reference to the method of Scheme T-3, the target compound (GT-08420) was obtained (white solid, 22 mg, yield 36%). ¹H NMR (400 MHz, DMSO-d₆) δ 11.63 (s, 1H), 11.31 (s, 1H), 10.70 (s, 1H), 8.75-8.66 (m, 1H), 8.53 (s, 1H), 8.47 (d, J=8.9 Hz, 1H), 8.38 (s, 1H), 8.22 (s, 1H), 7.99 (s, 1H), 7.94-7.85 (m, 2H), 7.83 (s, 1H), 7.50 (d, J=8.7 Hz, 1H), 5.95 (s, 1H), 5.24-5.13 (m, 1H), 4.75-4.55 (m, 4H), 4.45-4.40 (m, 2H), 3.79 (t, J=6.4 Hz, 3H), 3.23-3.14 (m, 2H), 2.89-2.72 (m, 2H), 2.55 (d, J=7.3 Hz, 2H), 2.15 (s, 3H), 1.55-1.47 (m, 8H), 0.88 (t, J=7.3 Hz, 3H). LCMS (ESI) calcd, for C₄₅H₅₃N₁₀O₅⁺ [M+H]⁺: 813.42, found, 813.3.

### Example 431: Preparation of 6-(6-(4-(((2-(2,4-dioxotetrahydropyrimidin-1(2H)-yl)-1,3-dioxoisoindolin-5-yl)methyl)(methyl)amino)piperidin-1-yl)pyridin-3-yl)-1-isopropyl-N-((6-methyl-2-oxo-4-propyl-1,2-dihydropyridin-3-yl)methyl)-1H-indazole-4-carboxamide (GT-08421)

With reference to the method of Scheme T-3, the target compound (GT-08421) was obtained (white solid, 21 mg, yield 34%). ¹H NMR (400 MHz, DMSO-d₆) δ 11.57 (d, J=23.4 Hz, 2H), 10.94 (s, 1H), 8.74-8.66 (m, 1H), 8.53 (s, 1H), 8.49 (d, J=8.4 Hz, 1H), 8.43 (d, J=8.2 Hz, 1H), 8.39 (s, 1H), 8.31 (d, J=5.2 Hz, 1H), 8.23 (s, 1H), 8.10 (d, J=7.7 Hz, 1H), 7.84 (s, 1H), 7.51 (d, J=8.9 Hz, 1H), 5.95 (s, 1H), 5.23-5.13 (m, 1H), 4.70 (s, 3H), 4.50-4.41 (m, 3H), 3.82 (t, J=6.8 Hz, 3H), 3.22 (s, 2H), 2.86 (t, J=6.7 Hz, 2H), 2.61 (d, J=3.6 Hz, 3H), 2.57-2.53 (m, 2H), 2.41-2.31 (m, 2H), 2.15 (s, 3H), 2.01-1.89 (m, 2H), 1.55-1.49 (m, 8H), 0.88 (t, J=7.3 Hz, 3H). LCMS (ESI) calcd, for C₄₅H₅₁N₁₀O₆⁺ [M+H]⁺: 827.40, found, 827.2.

### Example 432: Preparation of 6-(6-(4-(1-((2-(2,4-dioxotetrahydropyrimidin-1(2H)-yl)-1-oxoisoindolin-5-yl)methyl)piperidin-4-yl)piperazin-1-yl)pyridin-3-yl)-1-isopropyl-N-((6-methyl-2-oxo-4-propyl-1,2-dihydropyridin-3-yl)methyl)-1H-indazole-4-carboxamide (GT-08422)

With reference to the method of Scheme T-3, the target compound (GT-08422) was obtained (white solid, 30 mg, yield 55%). ¹H NMR (400 MHz, DMSO-d₆) δ 11.60 (s, 2H), 11.26 (s, 1H), 10.68 (s, 1H), 8.73-8.59 (m, 2H), 8.37 (s, 1H), 8.24 (d, J=8.9 Hz, 1H), 8.14 (s, 1H), 7.89-7.84 (m, 2H), 7.83-7.75 (m, 1H), 7.19 (d, J=9.0 Hz, 1H), 5.93 (s, 1H), 5.22-5.07 (m, 1H), 4.82 (d, J=17.0 Hz, 1H), 4.63-4.50 (m, 3H), 4.48-4.41 (m, 3H), 3.79 (t, J=6.8 Hz, 2H), 3.57-3.51 (m, 6H), 3.49-3.42 (m, 3H), 3.17 (s, 2H), 3.00 (s, 2H), 2.90-2.71 (m, 2H), 2.54 (d, J=9.8 Hz, 2H), 2.36 (t, J=11.0 Hz, 2H), 2.27-2.21 (m, 1H), 2.12 (s, 3H), 1.57-1.47 (m, 8H), 0.88 (t, J=7.3 Hz, 3H). LCMS (ESI) calcd, for C₄₈H₅₈N₁₁O₅⁺ [M+H]⁺: 868.46, found, 868.3.

### Example 433: Preparation of 6-(6-(4-(1-((2-(2,4-dioxotetrahydropyrimidin-1(2H)-yl)-1,3-dioxoisoindolin-5-yl)methyl)piperidin-4-yl)piperazin-1-yl)pyridin-3-yl)-1-isopropyl-N-((6-methyl-2-oxo-4-propyl-1,2-dihydropyridin-3-yl)methyl)-1H-indazole-4-carboxamide (GT-08423)

With reference to the method of Scheme T-3, the target compound obtained (GT-08423) was (white solid, 16 mg, yield 29%). ¹H NMR (400 MHz, DMSO-d₆) δ 11.65 (s, 2H), 11.41 (s, 1H), 10.93 (s, 1H), 8.69 (s, 1H), 8.64 (s, 1H), 8.37 (s, 1H), 8.31 (s, 1H), 8.24 (d, J=9.2 Hz, 1H), 8.18 (d, J=7.6 Hz, 1H), 8.15-8.09 (m, 2H), 7.84 (s, 1H), 7.19 (d, J=9.0 Hz, 1H), 5.93 (s, 1H), 5.20-5.10 (m, 1H), 4.55 (s, 4H), 4.42 (d, J=4.7 Hz, 2H), 3.83 (t, J=6.7 Hz, 3H), 3.51-3.43 (m, 4H), 3.17 (s, 3H), 3.03 (s, 2H), 2.86 (t, J=6.8 Hz, 2H), 2.54 (d, J=7.9 Hz, 2H), 2.41-2.33 (m, 2H), 2.28-2.19 (m, 2H), 2.14 (s, 3H), 1.54-1.49 (m, 8H), 0.88 (t, J=7.3 Hz, 3H). LCMS (ESI) calcd, for C₄₈H₅₆N₁₁O₆⁺ [M+H]⁺: 882.44, found, 882.2.

### Example 434: Preparation of N-((4,6-dimethyl-2-oxo-1,2-dihydropyridin-3-yl)methyl)-4′-((4-((2-(2,4-dioxotetrahydropyrimidin-1(2H)-yl)-1-oxoisoindolin-5-yl)methyl)piperazin-1-yl)methyl)-5-(ethyl(tetrahydro-2H-pyran-4-yl)amino)-4-methyl-[1,1′-biphenyl]-3-carboxamide (GT-08508)

With reference to the method of Scheme T-3, the target compound (GT-08508) was obtained (white solid, 29 mg, yield 46%). ¹H NMR (400 MHz, DMSO-d₆) δ 12.42 (s, 1H), 11.58 (s, 1H), 10.68 (d, J=8.7 Hz, 1H), 8.48 (s, 1H), 7.95 (s, 1H), 7.94-7.88 (m, 2H), 7.83 (s, 2H), 7.81-7.74 (m, 2H), 7.73-7.59 (m, 1H), 5.91 (s, 1H), 4.80 (d, J=16.7 Hz, 1H), 4.60-4.42 (m, 4H), 4.31 (d, J=4.9 Hz, 2H), 3.81-3.80 (m, 4H), 3.78-3.76 (m, 6H), 3.60-3.43 (m, 8H), 3.26 (s, 2H), 2.91-2.75 (m, 2H), 2.49-2.39 (m, 2H), 2.24 (s, 3H), 2.12 (s, 3H), 1.59 (d, 2H), 0.91 (s, 2H). LCMS (ESI) calcd, for C₄₇H₅₇N₈O₆⁺ [M+H]⁺: 829.44, found, 829.2.

### Example 435: Preparation of N-((4,6-dimethyl-2-oxo-1,2-dihydropyridin-3-yl)methyl)-4′-((4-((2-(2,4-dioxotetrahydropyrimidin-1(2H)-yl)-1,3-dioxoisoindolin-5-yl)methyl)piperazin-1-yl)methyl)-5-(ethyl(tetrahydro-2H-pyran-4-yl)amino)-4-methyl-[1,1′-biphenyl]-3-carboxamide (GT-08509)

With reference to the method of Scheme T-3, the target compound (GT-08509) was obtained (white solid, 36 mg, yield 57%). ¹H NMR (400 MHz, DMSO-d₆) δ 11.58 (s, 1H), 10.92 (s, 1H), 10.54 (d, J=54.5 Hz, 1H), 8.46 (s, 1H), 8.32 (d, 1H), 8.18 (d, 1H), 8.08 (d, J=7.7 Hz, 1H), 7.89 (s, 2H), 7.80 (d, 2H), 7.70 (s, 1H), 5.91 (s, 1H), 4.65-4.39 (m, 4H), 4.31 (d, J=4.7 Hz, 2H), 3.82 (s, 3H), 3.54-3.42 (m, 7H), 3.26 (s, 2H), 2.85 (t, J=6.8 Hz, 2H), 2.48-2.38 (m, 2H), 2.24 (s, 3H), 2.12 (s, 3H), 1.58 (s, 2H), 0.91 (s, 2H). LCMS (ESI) calcd. for C₄₇H₅₅N₈O₇⁺ [M+H]⁺: 843.42, found, 843.3.

### Example 436: Preparation of N-((4,6-dimethyl-2-oxo-1,2-dihydropyridin-3-yl)methyl)-4′-(4-((2-(2,4-dioxotetrahydropyrimidin-1(2H)-yl)-1-oxoisoindolin-5-yl)methyl)piperazin-1-yl)-5-(ethyl(tetrahydro-2H-pyran-4-yl)amino)-4-methyl-[1,1′-biphenyl]-3-carboxamide (GT-08510)

With reference to the method of Scheme T-3, the target compound (GT-08510) was obtained (white solid, 18 mg, yield 30%). ¹H NMR (400 MHz, DMSO-d₆) δ 11.82 (s, 1H), 11.62 (s, 1H), 10.68 (s, 1H), 10.47 (s, 1H), 8.46 (s, 1H), 7.97 (s, 1H), 7.89-7.83 (m, 2H), 7.76-7.64 (m, 2H), 7.62-7.52 (m, 1H), 7.08 (d, J=8.7 Hz, 2H), 5.91 (s, 1H), 4.80 (d, J=20.0 Hz, 1H), 4.57 (d, J=24.9 Hz, 3H), 4.30 (d, J=4.8 Hz, 2H), 3.91 (s, 2H), 3.86-3.83 (m, 6H), 3.82-3.77 (m, 4H), 3.44-3.37 (m, 2H), 3.33-3.14 (m, 6H), 2.90-2.74 (m, 2H), 2.49-2.37 (m, 3H), 2.23 (s, 3H), 2.13 (s, 3H), 1.64 (d, J=53.7 Hz, 2H), 0.92 (s, 2H). LCMS (ESI) calcd. for C₄₆H₅₅N₈O₆⁺ [M+H]⁺: 815.42, found, 815.3.

### Example 437: Preparation of N-((4,6-dimethyl-2-oxo-1,2-dihydropyridin-3-yl)methyl)-4′-(4-((2-(2,4-dioxotetrahydropyrimidin-1(2H)-yl)-1,3-dioxoisoindolin-5-yl)methyl)piperazin-1-yl)-5-(ethyl(tetrahydro-2H-pyran-4-yl)amino)-4-methyl-[1,1′-biphenyl]-3-carboxamide (GT-08511)

With reference to the method of Scheme T-3, the target compound (GT-08511) was obtained (white solid, 26 mg, yield 42%). ¹H NMR (400 MHz, DMSO-d₆) δ 12.08 (s, 1H), 11.58 (s, 1H), 10.93 (s, 1H), 8.47 (s, 1H), 8.39 (s, 1H), 8.26 (d, J=7.7 Hz, 1H), 8.12 (d, J=7.7 Hz, 1H), 8.04-7.88 (m, 1H), 7.72 (d, J=7.6 Hz, 2H), 7.58 (s, 1H), 7.08 (d, J=8.6 Hz, 2H), 5.92 (s, 1H), 4.64 (s, 2H), 4.30 (d, J=4.8 Hz, 2H), 4.00 (s, 2H), 3.82 (d, J=6.7 Hz, 6H), 3.79-3.72 (m, 2H), 3.42 (d, J=6.1 Hz, 2H), 3.33-3.11 (m, 7H), 2.87 (t, J=6.8 Hz, 2H), 2.43 (d, J=29.8 Hz, 3H), 2.23 (s, 3H), 2.13 (s, 3H), 1.65 (d, J=54.6 Hz, 2H), 0.96 (d, J=30.8 Hz, 3H). LCMS (ESI) calcd, for C₄₆H₅₃N₈O₇⁺ [M+H]⁺: 829.40, found, 829.2.

### Example 438: Preparation of N-((4,6-dimethyl-2-oxo-1,2-dihydropyridin-3-yl)methyl)-4′-(4-(((2-(2,4-dioxotetrahydropyrimidin-1(2H)-yl)-1-oxoisoindolin-5-yl)methyl)(methyl)amino)piperidin-1-yl)-5-(ethyl(tetrahydro-2H-pyran-4-yl)amino)-4-methyl-[1,1′-biphenyl]-3-carboxamide (GT-08512)

With reference to the method of Scheme T-3, the target compound (GT-08512) was obtained (white solid, 25 mg, yield 42%). ¹H NMR (400 MHz, DMSO-d₆) δ 11.41 (s, 1H), 10.68 (d, J=11.9 Hz, 1H), 8.46 (s, 1H), 8.00 (s, 1H), 7.86 (s, 2H), 7.79-7.70 (m, 2H), 7.69-7.52 (m, 2H), 7.29 (s, 2H), 5.92 (s, 1H), 4.87-4.76 (m, 1H), 4.66-4.55 (m, 2H), 4.41 (s, 1H), 4.31 (d, J=4.8 Hz, 2H), 4.07-4.03 (m, 4H), 3.79 (t, J=6.5 Hz, 4H), 3.51 (s, 1H), 3.20 (s, 3H), 2.99 (s, 1H), 2.93-2.73 (m, 3H), 2.63 (d, J=4.0 Hz, 3H), 2.43 (d, J=29.7 Hz, 3H), 2.36-2.26 (m, 2H), 2.24 (s, 3H), 2.13 (s, 3H), 1.64 (d, J=50.0 Hz, 2H), 1.32-1.26 (m, 3H), 0.92 (s, 2H). LCMS (ESI) calcd, for C₄₈H₅₉N₈O₆⁺ [M+H]⁺: 843.46, found, 843.3.

### Example 439: Preparation of N-((4,6-dimethyl-2-oxo-1,2-dihydropyridin-3-yl)methyl)-4′-(4-(((2-(2,4-dioxotetrahydropyrimidin-1(2H)-yl)-1,3-dioxoisoindolin-5-yl)methyl)(methyl)amino)piperidin-1-yl)-5-(ethyl(tetrahydro-2H-pyran-4-yl)amino)-4-methyl-[1,1′-biphenyl]-3-carboxamide (GT-08513)

With reference to the method of Scheme T-3, the target compound (GT-08513) was obtained (white solid, 23 mg, yield 38%). ¹H NMR (400 MHz, DMSO-d₆) δ 11.61 (s, 1H), 10.93 (s, 1H), 10.75-10.34 (m, 1H), 8.54-8.37 (m, 2H), 8.31 (t, J=7.1 Hz, 1H), 8.10 (d, J=7.7 Hz, 1H), 8.05-7.87 (m, 1H), 7.76 (d, J=6.9 Hz, 2H), 7.59 (s, 1H), 7.31 (s, 2H), 5.92 (s, 1H), 4.72 (d, J=13.1 Hz, 1H), 4.49 (s, 1H), 4.31 (d, J=4.7 Hz, 2H), 4.06-4.01 (m, 6H), 3.85-3.80 (m, 4H), 3.69-3.50 (m, 3H), 3.39-3.14 (m, 3H), 3.02 (s, 2H), 2.86 (t, J=6.8 Hz, 2H), 2.63 (s, 3H), 2.47 (s, 2H), 2.42-2.31 (m, 3H), 2.24 (s, 3H), 2.13 (s, 3H), 1.65 (d, J=56.4 Hz, 2H), 0.92 (s, 2H). LCMS (ESI) calcd, for C₄₈H₅₇N₈O₇⁺ [M+H]⁺: 857.43, found, 857.3.

### Example 440: Preparation of N-((4,6-dimethyl-2-oxo-1,2-dihydropyridin-3-yl)methyl)-4′-(4-(1-((2-(2,4-dioxotetrahydropyrimidin-1(2H)-yl)-1-oxoisoindolin-5-yl)methyl)piperidin-4-yl)piperazin-1-yl)-5-(ethyl(tetrahydro-2H-pyran-4-yl)amino)-4-methyl-[1,1′-biphenyl]-3-carboxamide (GT-08514)

With reference to the method of Scheme T-3, the target compound (GT-08514) was obtained (white solid, 29 mg, yield 49%). ¹H NMR (400 MHz, DMSO-d₆) δ 11.72-11.29 (m, 2H), 10.67 (d, J=17.6 Hz, 1H), 8.46 (s, 1H), 7.95 (d, J=22.3 Hz, 1H), 7.92-7.85 (m, 2H), 7.81 (d, J=7.9 Hz, 1H), 7.72 (s, 1H), 7.59 (s, 1H), 7.10 (d, J=8.5 Hz, 2H), 5.91 (s, 1H), 4.82 (d, J=16.6 Hz, 1H), 4.58 (d, J=16.6 Hz, 1H), 4.47 (s, 2H), 4.30 (d, J=4.6 Hz, 2H), 4.00 (s, 2H), 3.94 (d, J=10.2 Hz, 3H), 3.81-3.77 (m, 4H), 3.71-3.66 (m, 4H), 3.57-3.50 (m, 4H), 3.31-3.19 (m, 5H), 3.03 (s, 2H), 2.91-2.75 (m, 2H), 2.38 (s, 3H), 2.33-2.25 (m, 2H), 2.23 (s, 3H), 2.13 (s, 3H), 1.65 (d, J=52.0 Hz, 2H), 1.33-1.24 (m, 3H), 0.92 (s, 2H). LCMS (ESI) calcd, for C₅₁H₆₄N₉O₆⁺ [M+H]⁺: 898.50, found, 898.3.

### Example 441: Preparation of N-((4,6-dimethyl-2-oxo-1,2-dihydropyridin-3-yl)methyl)-4′-(4-(1-((2-(2,4-dioxotetrahydropyrimidin-1(2H)-yl)-1,3-dioxoisoindolin-5-yl)methyl)piperidin-4-yl)piperazin-1-yl)-5-(ethyl(tetrahydro-2H-pyran-4-yl)amino)-4-methyl-[1,1′-biphenyl]-3-carboxamide (GT-08515)

With reference to the method of Scheme T-3, the target compound (GT-08515) was obtained (white solid, 30 mg, yield 49%). ¹H NMR (400 MHz, DMSO-d₆) δ 11.59 (d, J=65.2 Hz, 2H), 10.93 (s, 1H), 10.48 (s, 1H), 8.60-8.36 (m, 1H), 8.32 (s, 1H), 8.19 (d, J=7.7 Hz, 1H), 8.11 (d, J=7.7 Hz, 1H), 8.06-7.89 (m, 1H), 7.88-7.62 (m, 2H), 7.57 (d, J=14.6 Hz, 1H), 7.10 (d, J=8.3 Hz, 2H), 5.90 (s, 1H), 4.56 (s, 1H), 4.30 (d, J=4.8 Hz, 2H), 4.02-3.90 (m, 3H), 3.83 (t, J=6.7 Hz, 4H), 3.69 (s, 4H), 3.55 (s, 6H), 3.33-3.16 (m, 6H), 3.04 (s, 2H), 2.86 (t, J=6.8 Hz, 2H), 2.43-2.35 (m, 3H), 2.31-2.17 (m, 6H), 2.12 (s, 3H), 1.76-1.43 (m, 2H), 1.30-1.21 (m, 1H), 0.96 (d, J=35.2 Hz, 3H). LCMS (ESI) calcd, for C₅₁H₆₂N₉O₇⁺ [M+H]⁺: 912.48, found, 912.3.

### Example 442: Preparation of 6-(6-(4-((1-(2-(2,4-dioxotetrahydropyrimidin-1(2H)-yl)-1,3-dioxoisoindolin-5-yl)piperidin-4-yl)methyl)piperazin-1-yl)pyridin-3-yl)-1-isopropyl-N-((6-methyl-2-oxo-4-propyl-1,2-dihydropyridin-3-yl)methyl)-1H-indazole-4-carboxamide (GT-08516)

With reference to the method of Scheme T-3, the target compound (GT-08516) was obtained (yellow solid, 39 mg, yield 56%). ¹H NMR (400 MHz, DMSO-d₆) δ 11.64 (s, 1H), 10.93 (s, 1H), 10.85 (s, 1H), 8.68 (d, J=2.0 Hz, 2H), 8.38 (s, 1H), 8.31 (d, J=8.6 Hz, 1H), 8.15 (s, 1H), 7.85 (s, 1H), 7.73 (d, J=8.6 Hz, 1H), 7.42 (d, J=2.0 Hz, 1H), 7.34-7.30 (m, 1H), 7.27 (d, J=9.0 Hz, 1H), 5.95 (s, 1H), 5.18-5.11 (m, 1H), 4.51 (d, J=13.3 Hz, 2H), 4.43 (d, J=4.7 Hz, 2H), 4.13 (d, J=13.0 Hz, 2H), 3.78 (t, J=5.9 Hz, 3H), 3.68-3.62 (m, 4H), 3.14 (d, J=9.2 Hz, 1H), 3.11-3.04 (m, 4H), 2.84 (t, J=8.0, 6.7 Hz, 2H), 2.56-2.53 (m, 2H), 2.25 (s, 1H), 2.15 (s, 3H), 1.99 (d, J=11.3 Hz, 2H), 1.53-1.49 (m, 8H), 1.32-1.23 (m, 2H), 0.88 (d, J=7.4 Hz, 3H). LCMS (ESI) calcd, for C₄₈H₅₆N₁₁O₆⁺ [M+H]⁺: 882.44, found, 882.3.

### Example 443: Preparation of 6-(6-(4-((1-(2-(2,4-dioxotetrahydropyrimidin-1(2H)-yl)-1,3-dioxoisoindolin-4-yl)piperidin-4-yl)methyl)piperazin-1-yl)pyridin-3-yl)-1-isopropyl-N-((6-methyl-2-oxo-4-propyl-1,2-dihydropyridin-3-yl)methyl)-1H-indazole-4-carboxamide (GT-08517)

With reference to the method of Scheme T-3, the target compound (GT-08517) was obtained (yellow solid, 23 mg, yield 33%). ¹H NMR (400 MHz, DMSO-d₆) δ 11.61 (s, 1H), 10.86 (s, 2H), 8.76-8.61 (m, 2H), 8.37 (s, 1H), 8.28 (d, J=7.7 Hz, 1H), 8.20-8.10 (m, 1H), 7.85 (s, 1H), 7.79-7.70 (m, 1H), 7.41 (t, J=8.5 Hz, 2H), 7.24 (d, J=9.1 Hz, 1H), 5.94 (s, 1H), 5.25-5.09 (m, 1H), 4.49 (d, J=13.3 Hz, 2H), 4.43 (d, J=4.8 Hz, 2H), 3.85-3.81 (m, 4H), 3.71-3.55 (m, 8H), 3.14 (s, 3H), 3.05-2.91 (m, 2H), 2.88-2.76 (m, 2H), 2.55 (d, J=7.6 Hz, 2H), 2.15 (s, 3H), 2.03 (s, 2H), 1.57-1.49 (m, 8H), 0.89 (t, J=7.3 Hz, 3H). LCMS (ESI) calcd. for C₄₈H₅₆N₁₁O₆⁺ [M+H]⁺: 882.44, found, 882.3.

### Example 444: Preparation of N-((4,6-dimethyl-2-oxo-1,2-dihydropyridin-3-yl)methyl)-4′-((4-((1-(2-(2,4-dioxotetrahydropyrimidin-1(2H)-yl)-1,3-dioxoisoindolin-5-yl)piperidin-4-yl)methyl)piperazin-1-yl)methyl)-5-(ethyl(tetrahydro-2H-pyran-4-yl)amino)-4-methyl-[1,1′-biphenyl]-3-carboxamide (GT-08518)

With reference to the method of Scheme T-3, the target compound (GT-08518) was obtained (yellow solid, 35 mg, yield 50%). ¹H NMR (400 MHz, DMSO-d₆) δ 12.89 (s, 1H), 11.58 (s, 1H), 10.84 (s, 1H), 10.56 (s, 1H), 8.46 (s, 1H), 7.90 (s, 1H), 7.81 (d, J=7.0 Hz, 2H), 7.72 (d, J=8.6 Hz, 2H), 7.41 (s, 1H), 7.31 (d, J=8.6 Hz, 1H), 5.91 (s, 1H), 4.46 (s, 1H), 4.31 (d, J=4.8 Hz, 2H), 4.11 (d, J=12.2 Hz, 2H), 3.81 (s, 4H), 3.77 (t, J=6.8 Hz, 4H), 3.72 (s, 2H), 3.63-3.41 (m, 8H), 3.26 (s, 2H), 3.00 (t, J=12.3 Hz, 4H), 2.82 (t, J=15.9, 8.9 Hz, 2H), 2.48-2.35 (m, 2H), 2.24 (s, 3H), 2.18-2.10 (m, 4H), 1.98 (d, J=12.0 Hz, 2H), 1.61 (s, 2H), 1.27 (d, J=11.2 Hz, 2H), 0.91 (s, 2H). LCMS (ESI) calcd, for C₅₂H₆₄N₉O₇⁺ [M+H]⁺: 926.49, found, 926.3.

### Example 445: Preparation of N-((4,6-dimethyl-2-oxo-1,2-dihydropyridin-3-yl)methyl)-4′-((4-((1-(2-(2,4-dioxotetrahydropyrimidin-1(2H)-yl)-1,3-dioxoisoindolin-4-yl)piperidin-4-yl)methyl)piperazin-1-yl)methyl)-5-(ethyl(tetrahydro-2H-pyran-4-yl)amino)-4-methyl-[1,1′-biphenyl]-3-carboxamide (GT-08519)

With reference to the method of Scheme T-3, the target compound (GT-08519) was obtained (yellow solid, 31 mg, yield 45%). ¹H NMR (400 MHz, DMSO-d₆) δ 13.06-11.83 (m, 1H), 11.56 (s, 2H), 10.85 (s, 1H), 8.46 (s, 1H), 7.90 (s, 1H), 7.79 (s, 2H), 7.78-7.68 (m, 2H), 7.40 (t, J=7.5 Hz, 2H), 5.90 (s, 1H), 4.50 (s, 2H), 4.31 (d, J=4.8 Hz, 2H), 3.84-3.75 (m, 6H), 3.62-3.56 (m, 5H), 3.26 (s, 3H), 3.12 (s, 2H), 2.99-2.87 (m, 2H), 2.86-2.79 (m, 2H), 2.38 (d, J=40.1 Hz, 2H), 2.24 (s, 3H), 2.12 (s, 3H), 2.04 (s, 3H), 1.60 (s, 2H), 1.51-1.34 (m, 2H), 0.91 (s, 2H). LCMS (ESI) calcd, for C₅₂H₆₄N₉O₇⁺ [M+H]⁺: 926.49, found, 926.3.

### Example 446: Preparation of N-((4,6-dimethyl-2-oxo-1,2-dihydropyridin-3-yl)methyl)-4′-(4-((1-(2-(2,4-dioxotetrahydropyrimidin-1(2H)-yl)-1,3-dioxoisoindolin-5-yl)piperidin-4-yl)methyl)piperazin-1-yl)-5-(ethyl(tetrahydro-2H-pyran-4-yl)amino)-4-methyl-[1,1′-biphenyl]-3-carboxamide (GT-08520)

With reference to the method of Scheme T-3, the target compound (GT-08520) was obtained (yellow solid, 38 mg, yield 56%). ¹H NMR (400 MHz, DMSO-d₆) δ 11.56 (s, 1H), 10.84 (s, 1H), 10.38 (s, 1H), 8.46 (s, 1H), 8.02 (s, 1H), 7.73 (d, J=8.6 Hz, 2H), 7.63-7.48 (m, 1H), 7.42 (d, J=1.9 Hz, 1H), 7.33 (d, J=8.8 Hz, 1H), 7.11 (d, J=8.4 Hz, 2H), 5.90 (s, 1H), 4.30 (d, J=4.8 Hz, 2H), 4.13 (d, J=13.3 Hz, 2H), 3.88 (d, J=12.1 Hz, 3H), 3.80-3.76 (m, 4H), 3.70 (s, 6H), 3.62 (d, J=11.6 Hz, 3H), 3.39-3.31 (m, 2H), 3.26 (s, 1H), 3.16 (d, J=9.9 Hz, 2H), 3.11-2.99 (m, 4H), 2.83 (t, J=7.2 Hz, 2H), 2.46-2.33 (m, 2H), 2.23 (s, 4H), 2.12 (s, 3H), 1.98 (d, J=12.8 Hz, 2H), 1.64 (d, J=37.4 Hz, 2H), 1.37-1.24 (m, 2H), 0.92 (s, 2H). LCMS (ESI) calcd. for C₅₁H₆₂N₉O₇⁺ [M+H]⁺: 912.48, found, 912.2.

### Example 447: Preparation of N-((4,6-dimethyl-2-oxo-1,2-dihydropyridin-3-yl)methyl)-4′-(4-((1-(2-(2,4-dioxotetrahydropyrimidin-1(2H)-yl)-1,3-dioxoisoindolin-4-yl)piperidin-4-yl)methyl)piperazin-1-yl)-5-(ethyl(tetrahydro-2H-pyran-4-yl)amino)-4-methyl-[1,1′-biphenyl]-3-carboxamide (GT-08521)

With reference to the method of Scheme T-3, the target compound (GT-08521) was obtained (yellow solid, 26 mg, yield 39%). ¹H NMR (400 MHz, DMSO-d₆) δ 11.58 (s, 1H), 10.89 (d, J=27.1 Hz, 2H), 10.45 (s, 1H), 8.46 (s, 1H), 8.09 (s, 1H), 7.77-7.67 (m, 2H), 7.59 (s, 1H), 7.41 (t, J=8.7 Hz, 2H), 7.12 (d, J=8.6 Hz, 2H), 5.91 (s, 1H), 4.31 (d, J=4.8 Hz, 2H), 3.99 (s, 2H), 3.88 (d, J=12.7 Hz, 3H), 3.79-3.78 (m, 3H), 3.76-3.73 (m, 6H), 3.64 (d, J=10.6 Hz, 3H), 3.45-3.36 (m, 2H), 3.31-3.10 (m, 6H), 3.02-2.88 (m, 2H), 2.88-2.77 (m, 2H), 2.43 (d, J=27.5 Hz, 2H), 2.24 (s, 3H), 2.13 (s, 4H), 2.04 (s, 2H), 1.88-1.57 (m, 2H), 1.55-1.39 (m, 2H), 0.92 (s, 2H). LCMS (ESI) calcd, for C₅₁H₆₂N₉O₇⁺ [M+H]⁺: 912.48, found, 912.2.

### Example 448: Preparation of N-((4,6-dimethyl-2-oxo-1,2-dihydropyridin-3-yl)methyl)-4′-(4-(((1-(2-(2,4-dioxotetrahydropyrimidin-1(2H)-yl)-1,3-dioxoisoindolin-5-yl)piperidin-4-yl)methyl)(methyl)amino)piperidin-1-yl)-5-(ethyl(tetrahydro-2H-pyran-4-yl)amino)-4-methyl-[1,1′-biphenyl]-3-carboxamide (GT-08522)

With reference to the method of Scheme T-3, the target compound (GT-08522) was obtained (yellow solid, 30 mg, yield 46%). ¹H NMR (400 MHz, DMSO-d₆) δ 11.59 (s, 1H), 10.84 (s, 1H), 10.49 (s, 1H), 8.46 (s, 1H), 8.01 (s, 1H), 7.84-7.64 (m, 3H), 7.59 (s, 1H), 7.41 (d, J=1.8 Hz, 1H), 7.32 (d, J=8.8 Hz, 1H), 7.27 (s, 1H), 5.91 (s, 1H), 4.30 (d, J=4.9 Hz, 2H), 4.13 (d, J=12.3 Hz, 2H), 3.90 (s, 6H), 3.82-3.75 (m, 8H), 3.58-3.39 (m, 2H), 3.25 (s, 2H), 3.14 (s, 1H), 3.07-2.95 (m, 3H), 2.95-2.89 (m, 1H), 2.83 (t, J=7.2 Hz, 2H), 2.76 (d, J=4.6 Hz, 2H), 2.43 (d, J=26.1 Hz, 2H), 2.27 (s, 1H), 2.23 (s, 3H), 2.18 (s, 3H), 2.13 (s, 3H), 2.06-1.86 (m, 3H), 1.65 (d, J=51.6 Hz, 2H), 1.37-1.20 (m, 2H), 0.92 (s, 2H). LCMS (ESI) calcd, for C₅₃H₆₆N₉O₇⁺ [M+H]⁺: 940.51, found, 940.3.

### Example 449: Preparation of N-((4,6-dimethyl-2-oxo-1,2-dihydropyridin-3-yl)methyl)-4′-(4-(((1-(2-(2,4-dioxotetrahydropyrimidin-1(2H)-yl)-1,3-dioxoisoindolin-4-yl)piperidin-4-yl)methyl)(methyl)amino)piperidin-1-yl)-5-(ethyl(tetrahydro-2H-pyran-4-yl)amino)-4-methyl-[1,1′-biphenyl]-3-carboxamide (GT-08523)

With reference to the method of Scheme T-3, the target compound (GT-08523) was obtained (yellow solid, 27 mg, yield 41%). ¹H NMR (400 MHz, DMSO-d₆) δ 11.57 (s, 1H), 10.86 (s, 1H), 10.46 (s, 1H), 8.46 (s, 1H), 8.01 (s, 1H), 7.86-7.66 (m, 3H), 7.59 (s, 1H), 7.45-7.36 (m, 2H), 7.27 (s, 2H), 5.91 (s, 1H), 4.31 (d, J=4.8 Hz, 2H), 4.02 (s, 2H), 3.93 (s, 4H), 3.78-3.67 (m, 6H), 3.49 (s, 2H), 3.24 (s, 3H), 3.10-2.90 (m, 5H), 2.87-2.74 (m, 5H), 2.53 (s, 2H), 2.43 (d, J=27.7 Hz, 2H), 2.33-2.25 (m, 2H), 2.24 (s, 3H), 2.22-2.19 (m, 1H), 2.13 (s, 3H), 2.07-1.81 (m, 4H), 1.73-1.32 (m, 4H), 0.92 (s, 2H). LCMS (ESI) calcd, for C₅₃H₆₆N₉O₇⁺ [M+H]⁺: 940.51, found, 940.3.

### Example 450: Preparation of 4-(((4-(5-chloro-2-((1-((1-(2-(2,4-dioxotetrahydropyrimidin-1(2H)-yl)-1,3-dioxoisoindolin-5-yl)piperidin-4-yl)methyl)piperidin-4-yl)amino)pyridin-4-yl) thiazol-2-yl)amino)methyl)tetrahydro-2H-pyran-4-carbonitrile (GT-04719)

With reference to the method of Scheme T-3, the target compound (GT-04719) was obtained (yellow solid, 16 mg, yield 25.56%). ¹H NMR (400 MHz, DMSO-d₆) δ 10.84 (s, 1H), 10.38 (d, J=64.9 Hz, 1H), 8.36 (s, 1H), 8.07 (d, J=20.4 Hz, 1H), 7.72 (d, J=8.6 Hz, 1H), 7.69-7.61 (m, 1H), 7.54 (s, 1H), 7.41 (s, 1H), 7.34-7.26 (m, 1H), 4.13-4.10 (m, 2H), 3.94 (d, J=10.2 Hz, 4H), 3.78 (t, J=6.8 Hz, 3H), 3.71 (d, J=5.8 Hz, 2H), 3.61 (d, J=10.4 Hz, 2H), 3.51-3.41 (m, 3H), 3.06-2.97 (m, 4H), 2.83 (t, J=6.8 Hz, 2H), 2.22-2.03 (m, 4H), 1.92 (dd, J=29.2, 12.4 Hz, 4H), 1.73 (dd, J=17.1, 8.5 Hz, 2H), 1.37-1.19 (m, 2H). LCMS (ESI) calcd. for C₃₈H₄₄ClN₁₀O_{5S}⁺ [M+H]⁺: 787.29, found, 787.2.

### Example 451: Preparation of 4-(((4-(5-chloro-2-((1-((1-(2-(2,4-dioxotetrahydropyrimidin-1(2H)-yl)-1,3-dioxoisoindolin-4-yl)piperidin-4-yl)methyl)piperidin-4-yl)amino)pyridin-4-yl) thiazol-2-yl)amino)methyl)tetrahydro-2H-pyran-4-carbonitrile (GT-08345)

With reference to the method of Scheme T-3, the target compound (GT-08345) was obtained (yellow solid, 20 mg, yield 32%). ¹H NMR (400 MHz, DMSO-d₆) δ 10.86 (s, 1H), 10.34 (d, J=64.7 Hz, 1H), 8.35 (s, 1H), 8.07 (d, J=19.0 Hz, 1H), 7.78-7.72 (m, 1H), 7.64 (d, J=16.0 Hz, 1H), 7.52 (s, 1H), 7.40 (t, J=7.8 Hz, 2H), 3.93 (d, J=12.2 Hz, 3H), 3.79 (d, 4H), 3.71 (d, J=5.8 Hz, 4H), 3.48-3.41 (m, 3H), 3.02 (s, 4H), 2.97-2.89 (m, 2H), 2.86-2.81 (m, 2H), 2.18-2.05 (m, 4H), 2.01 (d, J=12.0 Hz, 2H), 1.89 (d, J=13.4 Hz, 2H), 1.76-1.67 (m, 2H), 1.51-1.35 (m, 2H). LCMS (ESI) calcd, for C₃₈H₄₄ClN₁₀O_{5S}⁺ [M+H]⁺: 787.29, found, 787.2.

### Example 452: Preparation of 5-(4-((4-((5-chloro-4-(5-(cyclopropylmethyl)-1-methyl-1H-pyrazol-4-yl)pyrimidin-2-yl)amino)piperidin-1-yl)methyl)piperidin-1-yl)-2-(2,4-dioxotetrahydropyrimidin-1(2H)-yl)isoindoline-1,3-dione (GT-08346)

With reference to the method of Scheme T-3, the target compound (GT-08346) was obtained (yellow solid, 9 mg, yield 16%). ¹H NMR (400 MHz, DMSO-d₆) δ 10.87 (s, 1H), 10.14 (d, J=83.1 Hz, 1H), 8.35 (d, J=12.7 Hz, 1H), 8.00 (s, 1H), 7.72 (d, J=8.6 Hz, 1H), 7.61 (s, 1H), 7.41 (s, 1H), 7.34-7.20 (m, 1H), 4.11 (d, J=12.8 Hz, 2H), 3.86 (s, 3H), 3.58 (d, J=11.4 Hz, 1H), 3.34-3.23 (m, 1H), 3.06-3.01 (m, 3H), 2.98 (d, J=8.4 Hz, 3H), 2.83 (t, J=7.0 Hz, 2H), 2.18 (s, 1H), 2.08-2.04 (m, 1H), 1.98-1.89 (m, 3H), 1.27 (d, J=11.8 Hz, 2H), 0.93 (s, 1H), 0.38 (s, 2H), 0.12 (d, J=16.5 Hz, 2H). LCMS (ESI) calcd, for C₃₅H₄₂ClN₁₀O₄⁺ [M+H]⁺: 701.31, found, 701.2.

### Example 453: Preparation of 4-(4-((4-((5-chloro-4-(5-(cyclopropylmethyl)-1-methyl-1H-pyrazol-4-yl)pyrimidin-2-yl)amino)piperidin-1-yl)methyl)piperidin-1-yl)-2-(2,4-dioxotetrahydropyrimidin-1(2H)-yl)isoindoline-1,3-dione (GT-08347)

With reference to the method of Scheme T-3, the target compound (GT-08347) was obtained (yellow solid, 20 mg, yield 35%). ¹H NMR (400 MHz, DMSO-d₆) δ 10.86 (s, 1H), 10.15 (d, J=84.2 Hz, 1H), 8.36 (d, J=12.8 Hz, 1H), 7.96 (s, 1H), 7.75 (t, 1H), 7.62 (s, 1H), 7.40 (t, J=7.4 Hz, 2H), 3.89 (s, 1H), 3.87 (d, J=2.4 Hz, 3H), 3.69 (d, J=12.1 Hz, 2H), 3.60 (d, J=9.9 Hz, 2H), 3.38-3.27 (m, 1H), 3.07-3.01 (m, 4H), 2.97-2.88 (m, 2H), 2.87-2.79 (m, 2H), 2.11-2.02 (m, 3H), 2.01-1.85 (m, 4H), 1.51-1.36 (m, 2H), 1.01-0.90 (m, 1H), 0.39 (s, 2H), 0.13 (d, J=16.4 Hz, 2H). LCMS (ESI) calcd, for C₃₅H₄₂ClN₁₀O₄⁺ [M+H]⁺: 701.31, found, 701.2.

### Example 454: Preparation of 4-(((5′-chloro-2′-((1-((1-(2-(2,4-dioxotetrahydropyrimidin-1(2H)-yl)-1,3-dioxoisoindolin-5-yl)piperidin-4-yl)methyl)piperidin-4-yl)amino)-[2,4′-bipyridin]-6-yl)amino)methyl)tetrahydro-2H-pyran-4-carbonitrile (GT-08348)

With reference to the method of Scheme T-3, the target compound (GT-08348) was obtained (yellow solid, 48 mg, yield 78%). ¹H NMR (400 MHz, DMSO-d₆) δ 10.84 (s, 1H), 10.45 (s, 1H), 8.12 (s, 1H), 7.71 (t, J=9.3 Hz, 3H), 7.41 (s, 1H), 7.34-7.29 (m, 1H), 7.03 (s, 1H), 6.96 (s, 1H), 6.90 (d, J=7.2 Hz, 1H), 4.10 (d, 4H), 3.92 (d, J=10.1 Hz, 3H), 3.78 (t, J=6.8 Hz, 3H), 3.73 (s, 2H), 3.60 (d, J=11.2 Hz, 2H), 3.50-3.42 (m, 3H), 3.05-2.97 (m, 5H), 2.82 (t, J=6.7 Hz, 2H), 2.16-2.09 (m, 2H), 1.96 (d, J=10.9 Hz, 2H), 1.88 (d, J=13.4 Hz, 2H), 1.69 (t, J=10.9 Hz, 2H), 1.33-1.24 (m, 2H). LCMS (ESI) calcd, for C₄₀H₄₆ClN₁₀O₅⁺ [M+H]⁺: 781.33, found, 781.2.

### Example 455: Preparation of 4-(((5′-chloro-2′-((1-((1-(2-(2,4-dioxotetrahydropyrimidin-1(2H)-yl)-1,3-dioxoisoindolin-4-yl)piperidin-4-yl)methyl)piperidin-4-yl)amino)-[2,4′-bipyridin]-6-yl)amino)methyl)tetrahydro-2H-pyran-4-carbonitrile (GT-08349)

With reference to the method of Scheme T-3, the target compound (GT-08349) was obtained (yellow solid, 35 mg, yield 57%). ¹H NMR (400 MHz, DMSO-d₆) δ 10.86 (s, 1H), 10.24 (d, J=39.8 Hz, 1H), 8.14 (d, J=16.0 Hz, 1H), 7.80-7.57 (m, 3H), 7.40 (t, J=7.5 Hz, 2H), 6.98-6.82 (m, 3H), 3.92 (s, 3H), 3.91-3.89 (m, 4H), 3.80-3.78 (m, 3H), 3.71 (s, 2H), 3.61 (d, J=11.0 Hz, 2H), 3.50-3.42 (m, 3H), 3.06-2.99 (m, 3H), 2.98-2.90 (m, 2H), 2.85-2.80 (m, 2H), 2.08-1.97 (m, 4H), 1.87 (d, J=13.3 Hz, 2H), 1.72-1.64 (m, 2H), 1.51-1.40 (m, 2H). LCMS (ESI) calcd, for C₄₀H₄₆ClN₁₀O₅⁺ [M+H]⁺: 781.33, found, 781.2.

### Example 456: Preparation of N-(5-cyclobutyl-1H-pyrazol-3-yl)-2-(4-(4-((1-(2-(2,4-dioxotetrahydropyrimidin-1(2H)-yl)-1,3-dioxoisoindolin-5-yl)piperidin-4-yl)methyl)piperazin-1-yl)phenyl)acetamide (GT-08350)

With reference to the method of Scheme T-3, the target compound (GT-08350) was obtained (yellow solid, 39 mg, yield 51%). ¹H NMR (400 MHz, DMSO-d₆) δ 10.84 (s, 1H), 10.78 (s, 1H), 10.53 (s, 1H), 7.76-7.70 (m, 1H), 7.41 (d, J=1.9 Hz, 1H), 7.34-7.29 (m, 1H), 7.23 (d, J=8.6 Hz, 2H), 6.96 (d, J=8.6 Hz, 2H), 6.29 (s, 1H), 4.13-4.10 (m, 3H), 3.81-3.72 (m, 6H), 3.58 (d, J=12.4 Hz, 2H), 3.53 (s, 2H), 3.30-3.24 (m, 2H), 3.14-3.10 (m, 1H), 3.08-3.03 (m, 3H), 2.83 (t, J=7.1 Hz, 2H), 2.29-2.18 (m, 3H), 2.13-2.05 (m, 2H), 1.99-1.89 (m, 3H), 1.85-1.78 (m, 1H), 1.34-1.21 (m, 2H). LCMS (ESI) calcd, for C₃₇H₄₄N₉O₅⁺ [M+H]⁺: 694.35, found, 694.3.

### Example 457: Preparation of N-(5-cyclobutyl-1H-pyrazol-3-yl)-2-(4-(4-((1-(2-(2,4-dioxotetrahydropyrimidin-1(2H)-yl)-1,3-dioxoisoindolin-4-yl)piperidin-4-yl)methyl)piperazin-1-yl)phenyl)acetamide (GT-08351)

With reference to the method of Scheme T-3, the target compound (GT-08351) was obtained (yellow solid, 30 mg, yield 39%). ¹H NMR (400 MHz, DMSO-d₆) δ 10.85 (s, 2H), 10.60 (s, 1H), 7.77-7.69 (m, 1H), 7.43-7.36 (m, 2H), 7.25 (d, J=11.8 Hz, 2H), 6.97 (d, J=8.7 Hz, 2H), 6.29 (s, 1H), 3.83-3.72 (m, 6H), 3.72-3.64 (m, 2H), 3.61 (d, J=11.3 Hz, 2H), 3.56-3.51 (m, 2H), 3.33-3.26 (m, 2H), 3.23-3.07 (m, 4H), 2.99-2.88 (m, 2H), 2.85-2.80 (m, 2H), 2.30-2.20 (m, 2H), 2.18-2.04 (m, 3H), 2.03-1.95 (m, 2H), 1.82-1.76 (m, 1H), 1.55-1.35 (m, 2H). LCMS (ESI) calcd, for C₃₇H₄₄N₉O₅⁺ [M+H]⁺: 694.35, found, 694.3.

### Example 458: Preparation of 4-(((4-(5-chloro-2-((1-((2-(2,4-dioxotetrahydropyrimidin-1(2H)-yl)-1-oxoisoindolin-5-yl)methyl)piperidin-4-yl)amino)pyridin-4-yl) thiazol-2-yl)amino)methyl)tetrahydro-2H-pyran-4-carbonitrile (GT-08153)

With reference to the method of Scheme T-3, the target compound (GT-08153) was obtained (white solid, 28 mg, yield 51%). ¹H NMR (400 MHz, DMSO-d₆) δ 11.33 (s, 1H), 10.68 (s, 1H), 8.34 (s, 1H), 8.03 (s, 1H), 7.97-7.91 (m, 1H), 7.88-7.82 (m, 2H), 7.65 (s, 1H), 7.48 (s, 1H), 4.82 (d, J=16.4 Hz, 1H), 4.58 (d, J=16.9 Hz, 1H), 4.46 (d, J=4.2 Hz, 2H), 3.94-3.91 (m, 4H), 3.69 (d, J=5.9 Hz, 2H), 3.50-3.41 (m, 5H), 3.10-2.99 (m, 2H), 2.91-2.84 (m, 1H), 2.81-2.73 (m, 1H), 2.15 (d, J=12.2 Hz, 2H), 2.04-1.95 (m, 2H), 1.87 (d, J=13.0 Hz, 2H), 1.75-1.67 (m, 2H). LCMS (ESI) calcd, for C₃₃H₃₇ClN₉O₄S⁺ [M+H]⁺: 690.24, found, 690.1.

### Example 459: Preparation of 4-(((4-(5-chloro-2-((1-((2-(2,4-dioxotetrahydropyrimidin-1(2H)-yl)-1,3-dioxoisoindolin-5-yl)methyl)piperidin-4-yl)amino)pyridin-4-yl) thiazol-2-yl)amino)methyl)tetrahydro-2H-pyran-4-carbonitrile (GT-08154)

With reference to the method of Scheme T-3, the target compound (GT-08154) was obtained (white solid, 34 mg, yield 61%). ¹H NMR (400 MHz, DMSO-d₆) δ 11.59 (s, 1H), 10.93 (s, 1H), 8.39-8.32 (m, 2H), 8.23 (d, J=7.9 Hz, 1H), 8.10 (d, J=7.7 Hz, 1H), 8.02 (s, 1H), 7.67 (s, 1H), 7.51 (s, 1H), 4.56 (s, 2H), 3.85-3.81 (m, 4H), 3.69 (d, J=5.7 Hz, 2H), 3.51-3.42 (m, 5H), 3.13-3.03 (m, 2H), 2.85 (t, J=6.7 Hz, 2H), 2.16 (d, J=11.1 Hz, 2H), 2.05-1.96 (m, 2H), 1.87 (d, J=13.1 Hz, 2H), 1.75-1.68 (m, 2H). LCMS (ESI) calcd. for C₃₃H₃₅ClN₉O₅S⁺ [M+H]⁺: 704.22, found, 704.1.

### Example 460: Preparation of 1-(5-((4-((5-chloro-4-(5-(cyclopropylmethyl)-1-methyl-1H-pyrazol-4-yl)pyrimidin-2-yl)amino)piperidin-1-yl)methyl)-1-oxoisoindolin-2-yl)dihydropyrimidine-2,4 (1H,3H)-dione (GT-08212)

With reference to the method of Scheme T-3, the target compound (GT-08212) was obtained (white solid, 45 mg, yield 68%). ¹H NMR (400 MHz, DMSO-d₆) δ 11.01 (d, J=55.5 Hz, 1H), 10.59 (s, 1H), 8.25 (s, 1H), 7.87 (s, 1H), 7.82 (s, 1H), 7.79 (d, J=7.8 Hz, 1H), 7.72 (d, J=7.9 Hz, 1H), 7.56 (s, 1H), 4.79-4.47 (m, 2H), 4.35 (s, 2H), 3.77 (s, 4H), 3.71 (t, J=6.8 Hz, 2H), 3.32 (s, 1H), 3.17 (s, 1H), 2.94 (s, 3H), 2.82-2.62 (m, 2H), 2.12-1.94 (m, 2H), 1.93-1.75 (m, 2H), 0.95-0.71 (m, 1H), 0.35-0.20 (m, 2H), 0.07-−0.06 (m, 2H). LCMS (ESI) calcd, for C₃₀H₃₅ClN₉O₃⁺ [M+H]⁺: 604.25, found, 604.2.

### Example 461: Preparation of 5-((4-((5-chloro-4-(5-(cyclopropylmethyl)-1-methyl-1H-pyrazol-4-yl)pyrimidin-2-yl)amino)piperidin-1-yl)methyl)-2-(2,4-dioxotetrahydropyrimidin-1(2H)-yl)isoindoline-1,3-dione (GT-08213)

With reference to the method of Scheme T-3, the target compound (GT-08213) was obtained (white solid, 44 mg, yield 65%). ¹H NMR (400 MHz, DMSO-d₆) δ 11.10 (d, J=77.0 Hz, 1H), 10.84 (s, 1H), 8.30-8.18 (m, 2H), 8.09 (d, J=7.7 Hz, 1H), 8.01 (d, J=7.6 Hz, 1H), 7.86 (s, 1H), 7.48 (s, 1H), 4.44 (s, 2H), 3.76 (s, 4H), 3.73 (t, J=6.8 Hz, 2H), 3.34 (d, J=10.8 Hz, 1H), 3.17 (s, 1H), 2.93 (s, 4H), 2.77 (t, J=6.7 Hz, 2H), 2.02 (dd, J=25.4, 12.7 Hz, 2H), 1.82 (t, J=12.4 Hz, 2H), 0.84 (s, 1H), 0.31-0.22 (m, 2H),−0.00 (s, 2H). LCMS (ESI) calcd, for C₃₀H₃₃ClN₉O₄⁺ [M+H]⁺: 618.23, found, 618.2.

### Example 462: Preparation of 4-(((5′-chloro-2′-((1-((2-(2,4-dioxotetrahydropyrimidin-1(2H)-yl)-1-oxoisoindolin-5-yl)methyl)piperidin-4-yl)amino)-[2,4′-bipyridin]-6-yl)amino)methyl)tetrahydro-2H-pyran-4-carbonitrile (GT-08214)

With reference to the method of Scheme T-3, the target compound (GT-08214) was obtained (white solid, 37 mg, yield 68%). ¹H NMR (400 MHz, DMSO-d₆) δ 11.28 (s, 1H), 10.69 (s, 1H), 8.11 (s, 1H), 7.93 (s, 1H), 7.90-7.76 (m, 3H), 7.71-7.64 (m, 1H), 6.96 (s, 2H), 6.88 (d, J=7.2 Hz, 1H), 4.82 (d, J=16.8 Hz, 1H), 4.58 (d, J=16.9 Hz, 1H), 4.45 (d, J=4.1 Hz, 2H), 4.00-3.97 (m, 1H), 3.90 (d, J=8.4 Hz, 2H), 3.79 (t, J=6.8 Hz, 2H), 3.72 (d, J=7.5 Hz, 2H), 3.45 (t, J=11.5 Hz, 4H), 3.06 (d, J=10.7 Hz, 2H), 2.91-2.83 (m, 1H), 2.80-2.72 (m, 1H), 2.15 (d, J=12.5 Hz, 2H), 2.02-1.93 (m, 2H), 1.87 (d, J=13.3 Hz, 2H), 1.73-1.65 (m, 2H). LCMS (ESI) calcd, for C₃₅H₃₉ClN₉O₄⁺ [M+H]⁺: 684.28, found, 684.2.

### Example 463: Preparation of 4-(((5′-chloro-2′-((1-((2-(2,4-dioxotetrahydropyrimidin-1(2H)-yl)-1,3-dioxoisoindolin-5-yl)methyl)piperidin-4-yl)amino)-[2,4′-bipyridin]-6-yl)amino)methyl)tetrahydro-2H-pyran-4-carbonitrile (GT-08215)

With reference to the method of Scheme T-3, the target compound (GT-08215) was obtained (white solid, 38 mg, yield 68%). ¹H NMR (400 MHz, DMSO-d₆) δ 11.33 (d, J=70.7 Hz, 1H), 10.93 (s, 1H), 8.34 (d, J=10.2 Hz, 1H), 8.20 (t, J=6.9 Hz, 1H), 8.17-8.01 (m, 3H), 7.66 (s, 1H), 6.94-6.84 (m, 3H), 4.54 (s, 2H), 3.92-3.88 (m, 3H), 3.82 (t, 2H), 3.70 (s, 2H), 3.48-3.41 (m, 4H), 3.12-3.02 (m, 2H), 2.85 (t, J=6.8 Hz, 2H), 2.15 (d, J=11.9 Hz, 2H), 1.97-1.89 (m, 2H), 1.86 (d, J=13.5 Hz, 2H), 1.71-1.65 (m, 2H). LCMS (ESI) calcd. for C₃₅H₃₇ClN₉O₅⁺ [M+H]⁺: 698.26, found, 698.1.

### Example 464: Preparation of N-(5-cyclobutyl-1H-pyrazol-3-yl)-2-(4-(4-(((2-(2,4-dioxotetrahydropyrimidin-1(2H)-yl)-1-oxoisoindolin-5-yl)methyl)(methyl)amino)piperidin-1-yl)phenyl)acetamide (GT-08228)

With reference to the method of Scheme T-3, the target compound (GT-08228) was obtained (white solid, 31 mg, yield 48%). ¹H NMR (400 MHz, DMSO-d₆) δ 11.58 (s, 1H), 10.94 (s, 1H), 10.69 (s, 1H), 8.02 (d, J=11.6 Hz, 1H), 7.96-7.85 (m, 2H), 7.61-7.51 (m, 2H), 7.41 (d, J=8.2 Hz, 2H), 6.30 (s, 1H), 4.82 (d, J=15.1 Hz, 2H), 4.62-4.54 (m, 3H), 4.48-4.44 (m, 1H), 3.80 (t, J=6.7 Hz, 2H), 3.74-3.71 (m, 1H), 3.65 (s, 2H), 3.60 (s, 1H), 3.50-3.45 (m, 1H), 3.40 (s, 1H), 2.90-2.84 (m, 1H), 2.80-2.74 (m, 1H), 2.66 (d, J=3.3 Hz, 3H), 2.42 (d, J=25.6 Hz, 3H), 2.30-2.21 (m, 2H), 2.14-2.05 (m, 2H), 1.98-1.90 (m, 1H), 1.86-1.73 (m, 1H). LCMS (ESI) calcd, for C₃₄H₄₁N₈O₄⁺ [M+H]⁺: 625.32, found, 625.2.

### Example 465: Preparation of N-(5-cyclobutyl-1H-pyrazol-3-yl)-2-(4-(4-(((2-(2,4-dioxotetrahydropyrimidin-1(2H)-yl)-1,3-dioxoisoindolin-5-yl)methyl)(methyl)amino)piperidin-1-yl)phenyl)acetamide (GT-08244)

With reference to the method of Scheme T-3, the target compound (GT-08244) was obtained (white solid, 35 mg, yield 52%). ¹H NMR (400 MHz, DMSO-d₆) δ 11.53 (s, 1H), 10.93 (s, 1H), 10.75 (s, 1H), 8.42 (d, J=15.8 Hz, 1H), 8.34-8.18 (m, 1H), 8.10 (d, J=7.7 Hz, 1H), 7.36 (s, 4H), 6.29 (s, 1H), 4.69 (s, 1H), 4.53 (s, 1H), 3.83 (t, J=6.7 Hz, 2H), 3.78 (s, 2H), 3.60 (s, 2H), 3.49-3.42 (m, 1H), 3.22 (s, 2H), 2.86 (t, J=6.7 Hz, 2H), 2.64 (s, 3H), 2.37 (d, J=12.4 Hz, 2H), 2.31-2.21 (m, 3H), 2.15-2.03 (m, 2H), 1.98-1.89 (m, 1H), 1.82 (t, J=9.3 Hz, 1H). LCMS (ESI) calcd, for C₃₄H₃₉N₈O₅⁺ [M+H]⁺: 639.30, found, 639.2.

### Example 466: Preparation of N-(5-cyclobutyl-1H-pyrazol-3-yl)-2-(4-(4-((2-(2,4-dioxotetrahydropyrimidin-1(2H)-yl)-1-oxoisoindolin-5-yl)methyl)piperazin-1-yl)phenyl)acetamide (GT-08229)

With reference to the method of Scheme T-3, the target compound (GT-08229) was obtained (white solid, 32 mg, yield 48%). ¹H NMR (400 MHz, DMSO-d₆) δ 11.54 (s, 1H), 10.81 (s, 1H), 10.69 (s, 1H), 7.94 (s, 1H), 7.90-7.80 (m, 2H), 7.22 (d, J=8.6 Hz, 2H), 6.93 (d, J=8.7 Hz, 2H), 6.29 (s, 1H), 4.81 (d, J=16.6 Hz, 1H), 4.56 (d, 1H), 4.51 (s, 2H), 3.83-3.73 (m, 4H), 3.52 (s, 2H), 3.49-3.44 (m, 1H), 3.37 (s, 2H), 3.18 (d, J=7.4 Hz, 3H), 2.91-2.82 (m, 1H), 2.80-2.71 (m, 1H), 2.30-2.19 (m, 2H), 2.14-2.03 (m, 2H), 1.99-1.89 (m, 1H), 1.86-1.77 (m, 1H). LCMS (ESI) calcd, for C₃₂H₃₇N₈O₄⁺ [M+H]⁺: 597.29, found, 597.2.

### Example 467: Preparation of N-(5-cyclobutyl-1H-pyrazol-3-yl)-2-(4-(4-((2-(2,4-dioxotetrahydropyrimidin-1(2H)-yl)-1,3-dioxoisoindolin-5-yl)methyl)piperazin-1-yl)phenyl)acetamide (GT-08230)

With reference to the method of Scheme T-3, the target compound (GT-08230) was obtained (white solid, 27 mg, yield 40%). ¹H NMR (400 MHz, DMSO-d₆) δ 11.74 (s, 1H), 10.92 (s, 1H), 10.80 (s, 1H), 8.36 (s, 1H), 8.22 (d, J=7.8 Hz, 1H), 8.10 (d, J=7.7 Hz, 1H), 7.22 (d, J=8.5 Hz, 2H), 6.93 (d, J=8.6 Hz, 2H), 6.29 (s, 1H), 4.60 (s, 2H), 3.82 (t, J=6.8 Hz, 2H), 3.80-3.70 (m, 2H), 3.52 (s, 2H), 3.49-3.44 (m, 1H), 3.38 (s, 2H), 3.26-3.05 (m, 4H), 2.86 (t, J=8.6, 4.9 Hz, 2H), 2.29-2.20 (m, 2H), 2.15-2.04 (m, 2H), 1.99-1.89 (m, 1H), 1.86-1.76 (m, 1H). LCMS (ESI) calcd, for C₃₂H₃₅N₈O₅⁺ [M+H]⁺: 611.27, found, 611.2.

### Example 468: Preparation of 5-((1R,4R)-2-oxa-5-azabicyclo[2.2.1]heptan-5-yl)-N-(3-(difluoromethyl)-1-((1r,4R)-4-((4-(2-(2,4-dioxotetrahydropyrimidin-1(2H)-yl)-4-fluoro-1,3-dioxoisoindolin-5-yl)piperazin-1-yl)methyl)cyclohexyl)-1H-pyrazol-4-yl)pyrazolo[1,5-a]pyrimidine-3-carboxamide (GT-05827)

With reference to the method of Scheme T-3, the target compound (GT-05827) was obtained (yellow solid, 17 mg, yield 24.84%). ¹H NMR (400 MHz, DMSO-d₆) δ 10.91 (s, 1H), 10.04 (s, 1H), 9.52 (d, J=6.6 Hz, 1H), 8.79 (d, J=7.7 Hz, 1H), 8.41 (d, J=4.1 Hz, 1H), 8.26 (d, J=5.5 Hz, 1H), 7.77 (d, J=8.1 Hz, 1H), 7.55 (t, J=7.9 Hz, 1H), 7.31-6.97 (m, 1H), 6.67 (dd, J=164.4, 7.8 Hz, 1H), 5.18 (d, J=81.2 Hz, 1H), 4.77 (d, J=20.2 Hz, 1H), 4.25 (s, 1H), 3.83-3.59 (m, 9H), 3.45 (d, J=10.3 Hz, 3H), 3.32-3.19 (m, 4H), 3.10 (s, 1H), 2.84 (t, J=6.8 Hz, 2H), 2.15-1.76 (m, 9H), 1.23 (d, J=11.7 Hz, 2H). LCMS (ESI) calcd, for C₃₉H₄₂F₃N₁₂O₆⁺ [M+H]⁺: 831.33, found, 831.3.

### Biological Activity Assay

### Reagents and Materials

| Reagents and materials | Suppliers or Source |
|---|---|
| Multiple myeloma cell line: MM.1S | ATCC (American Type |
| | Culture Collection) |
| Human Malignant Meningioma Cell | ATCC |
| Line: JEKO-1 | |
| Human non-small cell lung cancer | ATCC |
| cell line: NCI-H1975 | |
| Human breast cancer cell line: MCF-7 | ATCC |
| Human gastric cancer cell line: MGC803 | Dalian Meilun |
| | Biotech Co., Ltd. |
| Human T lymphocytic leukemia cell line: | ATCC |
| Jurkat | |
| Human large cell immunoblastic lymphoma | Dalian Meilun |
| cell line: SR | Biotech Co., Ltd. |
| Human acute T lymphoblastic leukemia | Dalian Meilun |
| cell line: Molt-4 | Biotech Co., Ltd. |
| Human myeloid monocytic leukemia cell | ATCC |
| line: MV-4-11 | |
| Human multiple myeloma peripheral blood | ATCC |
| B lymphocyte cell line: RPMI-8226 | |
| Human non-small cell lung cancer | ATCC |
| cell line: NCI-H2228 | |
| Human acute lymphocytic leukemia T | Dalian Meilun |
| lymphocytes cell line: CCRF-CEM | Biotech Co., Ltd. |
| Human lung cancer cell line: H292 | Dalian Meilun |
| | Biotech Co., Ltd. |
| Human diffuse large B-cell lymphoma | Kyinno Biotechnology |
| cell line: TMD8 | Co., Ltd |
| Human breast cancer cell line: | Dalian Meilun |
| MDA-MB-231 | Biotech Co., Ltd. |
| Human prostate cancer cell line: | ATCC |
| NCI-H358 | |
| Human malignant melanoma cell | Dalian Meilun |
| line: A375 | Biotech Co., Ltd. |
| Human neuroblastoma cell line: | Dalian Meilun |
| SK-N-AS | Biotech Co., Ltd. |
| Human diffuse large B-cell | Dalian Meilun |
| lymphoma cell line: WSU-DLCL2 | Biotech Co., Ltd. |
| RPMI-1640 Medium | ATCC |
| DMEM Medium | Dalian Meilun |
| | Biotech Co., Ltd. |
| IMDM Medium | Gibco Company |
| EMEM Medium | ATCC |
| Meilun Cell Counting Kit-8 | Dalian Meilun |
| | Biotech Co., Ltd. |
| Fetal bovine serum (FBS) | Sunrise Science |
| | Products Company |
| Penicillin-Streptomycin | Sunrise Science |
| | Products Company |
| CellTiter-Meiluncell Luminescent | Dalian Meilun |
| Cell Viability Assay Kit | Biotech Co., Ltd. |
| Mycoplasma detection kit | Sunrise Science |
| | Products Company |
| STR genotype detection | Shanghai Biowing Applied |
| | Biotechnology Co. Ltd. |

### Methods:

### Cell Cultures

The tumor cells used herein were cultured in a cell culture medium listed in table 3 at 37 °C in an incubator with 5% CO₂. Prior to experimentation, all cell lines used were correctly identified by STR profiling, and tested negative for mycoplasma contamination using a mycoplasma detection kit through routine screenings.

TABLE 3
Tumor cells and cell culture medium used in the present disclosure

| Cell line | Cell culture medium used |
|---|---|
| MM.1S | RPMI 1640 culture medium supplemented with 10% FBS, as well as |
| JEKO-1 | penicillin at a final concentration of 100 U/mL and streptomycin at a final |
| NCI-H1975 | concentration of 100 μg/mL |
| MGC803 | |
| Jurkat | |
| SR | |
| Molt-4 | |
| RPMI-8226 | |
| H2228 | |
| CCRF-CEM | |
| H292 | |
| TMD8 | |
| H358 | |
| WSU-DLCL2 | |
| MV-4-11 | IMDM culture medium supplemented with 10% FBS, as well as penicillin |
| | at a final concentration of 100 U/mL and streptomycin at a final |
| | concentration of 100 μg/mL |
| MDA-MB-231 | DMEM culture medium supplemented with 10% FBS, as well as penicillin |
| SK-N-AS | at a final concentration of 100 U/mL and streptomycin at a final |
| A375 | concentration of 100 μg/mL |
| MCF-7 | EMEM culture medium supplemented with 10% FBS, as well as penicillin |
| | at a final concentration of 100 U/mL and streptomycin at a final |
| | concentration of 100 μg/mL |

### I. Determination of Half Inhibitory Concentration (IC₅₀) of the Compounds Against Tumor Cells

The IC₅₀ values of the compounds of the present disclosure (including the compounds in Table 1, Table 2, and Examples 1-468) were determined using the CellTiter-Meiluncell Luminescent Cell Viability Assay Kit from Dalian Meilun Biotech Co., Ltd. The detailed procedure is as follows: Tumor cells were seeded in 96-well cell culture plates at the densities specified in Table 4. On the following day, the test compounds of the present disclosure were subjected to serial dilution. The dilution can be performed according to one of the following two methods:
(1) The first dilution method: The test compounds of the present disclosure (including the compounds in Table 1, Table 2, and Examples 1-468) were subjected to 5-fold serial dilutions starting from a highest concentration of 2000 μM, resulting in a total of 9 concentrations from high to low (2000, 400, 80, 16, 3.2, 0.64, 0.128, 0.0256, 0.00512 μM). Subsequently, 0.5 μL of the test compound dilution was transferred into the 96-well plate containing the cells, with each well containing 100 μL of cell culture medium. This resulted in final test compound concentrations of 10, 2, 0.4, 0.08, 0.016, 0.0032, 0.00064, 0.000128, and 0.0000256 μM, respectively, or
(2) As an alternative, a second dilution method can be used: The test compounds of the present disclosure (including the compounds in Table 1, Table 2, and Examples 1-468) were subjected to a 50-fold gradient dilution starting from a highest concentration of 100 μM, resulting in 2 concentrations (100 μM, 2 μM). Into the 96-well plate, the diluted test compounds and corresponding detection reagents were added, resulting in final test compound concentrations of 500 nM and 10 nM, respectively. The experimental procedure is described below using the diluted 2 μM test compound as an example.

0.5 μL of the aforementioned diluted compound of the present disclosure (including the compounds in Table 1, Table 2, and Examples 1-468) was added to the seeded cells in 100 μL of medium, yielding a final test compound concentration of 10 nM. After the cells were treated with the compounds of the present disclosure for a specified period, the cell viability assay reagent was added to the culture medium according to the kit's instructions to determine cell viability. The negative control was DMSO, and the reference controls were corresponding commercial inhibitors (including Orelabrutinib (CAS No.: 1655504-04-3); Ibrutinib (CAS No.: 936563-96-1); Compound GT-03308 (CAS No.: 1679327-21-9); Compound GT-03335 (CAS No.: 1801765-04-7); Compound GT-03336 (CAS No.: 2160546-07-4); Compound GT-03334 (CAS No.: 1801747-42-1); Compound TQB3804 (CAS No.: 2267329-76-8); Erlotinib (CAS No.: 183321-74-6); Brigatinib (CAS No.: 1197953-54-0); Alectinib (CAS No.: 1256580-46-7); ARV-471 (CAS No.: 2229711-08-2); Abemaciclib (CAS No.: 1231929-97-7); Ribociclib (CAS No.: 1211443-58-1); Palbociclib (CAS No.: 571190-30-2); Defactinib (CAS No.: 1073154-85-4); PND-1186 (CAS No.: 1061353-68-1); Dasatinib (CAS No.: 302962-49-8); Emavusertib (CAS No.: 1801344-14-8); AT7519 (CAS No.: 844442-38-2); Venetoclax (ABT-199, CAS No.: 1257044-40-8); PF-06650833 (CAS No.: 1817626-54-2); HJM-561 (CAS No.: 2570251-68-0); Osimertinib (CAS No.: 1421373-65-0); FN-1501 (CAS No.: 1429515-59-2); and ABT-263 (Navitoclax, CAS No.: 923564-51-6); KT-474 (CAS No.: 2432994-31-3); Afatinib (CAS No.: 850140-72-6); Enzalutamide (CAS No.: 915087-33-1); Tazemetostat (CAS No.: 1403254-99-8); UNC1999 (CAS No.: 1431612-23-5); AT7519 (CAS No.: 844442-38-2); and BI-D1870 (CAS No.: 501437-28-1)). All controls were treated using the same procedure as the compounds of the present disclosure. The growth inhibition curves for the compounds of the present disclosure were plotted using Prism GraphPad software, and the IC₅₀ values were calculated therefrom. The results were listed in Tables IA1 through IN4 below.

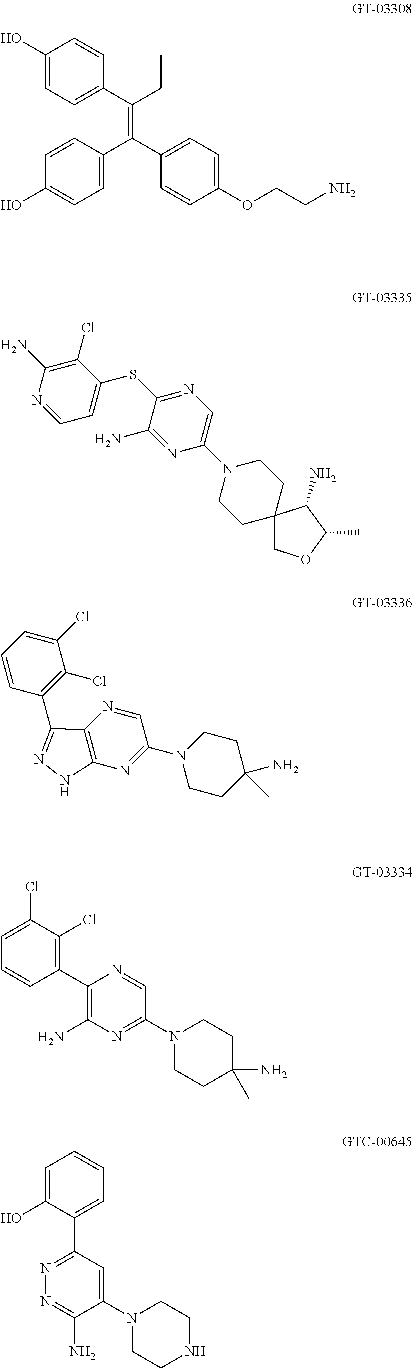 [SMILES: CC/C(=C(/c1ccc(O)cc1)c1ccc(OCCN)cc1)c1ccc(O)cc1 ; C[C@@H]1OCC2(CCN(c3cnc(Sc4ccnc(N)c4Cl)c(N)n3)CC2)[C@@H]1N ; CC1(N)CCN(c2cnc3c(-c4cccc(Cl)c4Cl)n[nH]c3n2)CC1 ; CC1(N)CCN(c2cnc(-c3cccc(Cl)c3Cl)c(N)n2)CC1 ; Nc1nnc(-c2ccccc2O)cc1N1CCNCC1]

TABLE 4
Seeding protocol and treatment time for tumor cells

| | | The times for treating cells with |
|---|---|---|
| | | the compounds of the present |
| Cells | Cells seeding protocol | disclosure (hours) |
| MM.1S | cells were seeded at a density of 10,000 cells per | 96 hours |
| Jurkat | well in 100 μL of serum-containing RPMI-1640 | |
| SR | medium per well | |
| RPMI-8226 | | |
| TMD8 | | |
| Molt-4 | cells were seeded at a density of 6,000 cells per well | 96 hours |
| | in 100 μL of serum-containing RPMI-1640 | |
| | medium per well | |
| JEKO-1 | cells were seeded at a density of 4,000 cells per well | 96 hours |
| WSU-DLCL2 | in 100 μL of serum-containing RPMI-1640 | |
| H358 | medium per well | |
| NCI-H1975 | | |
| H2228 | | |
| CCRF-CEM | | |
| H292 | | |
| MGC803 | cells were seeded at a density of 2,000 cells per well | 96 hours |
| | in 100 μL of serum-containing RPMI-1640 | |
| | medium per well | |
| MV-4-11 | cells were seeded at a density of 10,000 cells per | 96 hours |
| | well in 100 μL of serum-containing IMDM | |
| | medium per well | |
| MDA-MB-231 | cells were seeded at a density of 4,000 cells per well | 96 hours |
| A375 | in 100 μL of serum-containing DMEM medium per | |
| | well | |
| SK-N-AS | cells were seeded at a density of 3,000 cells per well | 96 hours |
| | in 100 μL of serum-containing DMEM medium per | |
| | well | |
| MCF-7 | cells were seeded at a density of 4,000 cells per well | 96 hours |
| | in 100 μL of serum-containing EMEM medium per | |
| | well | |

TABLE IA1
Inhibitory activity (IC₅₀, nM) of the compounds
of the present disclosure, designed based on the
IRAK4 inhibitors, on tumor cell proliferation

| | names | | | | |
|---|---|---|---|---|---|
| Comp. No. | MGC803 | Jurkat | SR | MM.1S | JEKO-1 |
| PF06650833 | E | E | D | F | C |
| GT-04880 | — | — | — | C | — |
| GT-04951 | A | A | C | A++ | A |
| GT-04953 | — | — | — | C | — |
| GT-04949 | C | C | A | A | A |
| GT-05019 | C | C | C | C | C |
| GT-05028 | C | A | C | A++ | B |

Note:
in table IA1, the symbols A+++, A++, A+, A, B, C, D, E, and F are defined as follows: 0 nM < A+++ < 10 nM; 10 nM < A++ ≤ 50 nM; 50 nM < A+ ≤ 100 nM; 100 nM < A ≤ 300 nM; 300 nM < B ≤ 500 nM; 500 nM < C ≤ 1000 nM; 1000 nM < D ≤ 5000 nM; 5000 nM < E ≤ 10000 nM; F > 10000 nM. The symbol “—” represents not tested.

TABLE IA2
Inhibitory activity of the compounds of the present disclosure, designed based on the IRAK4
inhibitors, on the Proliferation of Human Acute T Lymphoblastic Leukemia Cells (Molt-4)

| | Inhibition | Inhibition | | Inhibition | Inhibition | | Inhibition | Inhibition |
|---|---|---|---|---|---|---|---|---|
| | Rate at | Rate at | | Rate at | Rate at | | Rate at | Rate at |
| | 500 nM | 10 nM | Comp. | 500 nM | 10 nM | Comp. | 500 nM | 10 nM |
| Comp. No. | (%) | (%) | No. | (%) | (%) | No. | (%) | (%) |
| emavusertib | IAa2 | IAa3 | GT- | IAa9 | IAa4 | GT- | IAa6 | IAa6 |
| | | | 05749 | | | 08661 | | |
| KT-474 | IAa4 | IAa4 | GT- | IAa6 | IAa6 | GT- | IAa6 | IAa5 |
| | | | 08648 | | | 08663 | | |
| GT-05184 | IAa10 | IAa3 | GT- | IAa6 | IAa7 | GT- | IAa6 | IAa7 |
| | | | 08650 | | | 08664 | | |
| GT-05224 | IAa10 | IAa2 | GT- | IAa5 | IAa6 | GT- | IAa6 | IAa6 |
| | | | 08651 | | | 08665 | | |
| GT-05318 | IAa8 | IAa3 | GT- | IAa6 | IAa6 | GT- | IAa7 | IAa7 |
| | | | 08652 | | | 08667 | | |
| GT-05285 | IAa10 | IAa1 | GT- | IAa7 | IAa5 | GT- | IAa7 | IAa7 |
| | | | 08659 | | | 08669 | | |

Note:
in table IA2, the symbols IAa1, IAa2, IAa3, IAa4, IAa5, IAa6, IAa7, IAa8, IAa9, and IAa10 are defined as follows: −5% ≤ IAa1 ≤ 0, 0 < IAa2 < 3%, 3% ≤ IAa3 < 7%, 7% ≤ IAa4 < 10%, 10% ≤ IAa5 < 15%, 15% ≤ IAa6 < 20%, 20% ≤ IAa7 < 40%, 40%≤ IAa8 < 60%, 60% ≤ IAa9 < 80%, 80% ≤ IAa10 ≤ 100%.

TABLE IA3
Inhibitory activity of the compounds of the present disclosure, designed based on the IRAK4
inhibitors, on the Proliferation of Human myeloid monocytic leukemia cell line (MV-4-11)

| | Inhibition | Inhibition | | Inhibition | Inhibition | | Inhibition | Inhibition |
|---|---|---|---|---|---|---|---|---|
| | Rate at | Rate at | | Rate at | Rate at | | Rate at | Rate at |
| | 500 nM | 10 nM | Comp. | 500 nM | 10 nM | Comp. | 500 nM | 10 nM |
| Comp. No. | (%) | (%) | No. | (%) | (%) | No. | (%) | (%) |
| PF06650833 | IAb6 | IAb6 | GT-05285 | IAb11 | IAb8 | GT-05760 | IAb11 | IAb9 |
| GT-05184 | IAb11 | IAb9 | GT-05291 | IAb10 | IAb9 | GT-05803 | IAb10 | IAb9 |
| GT-05188 | IAb11 | IAb7 | GT-05746 | IAb10 | IAb5 | GT-05806 | IAb11 | IAb8 |
| GT-05224 | IAb11 | IAb9 | GT-05747 | IAb11 | IAb7 | GT-05816 | IAb9 | IAb2 |
| GT-05238 | IAb10 | IAb6 | GT-05749 | IAb11 | IAb4 | GT-05817 | IAb9 | IAb3 |
| GT-05318 | IAb12 | IAb11 | GT-05750 | IAb11 | IAb7 | GT-05818 | IAb9 | IAb5 |
| GT-05325 | IAb10 | IAb1 | | IAb11 | | | | |

Note:
in table IA3, the symbols IAb1, IAb2, IAb3, IAb4, IAb5, IAb6, IAb7, IAb8, IAb9, IAb10, IAb11, and IAb12 are defined as follows: −15% ≤ IAb1 < −10%, −10% ≤ IAb2 < −5%, −5% ≤ IAb3 ≤ 0%, 0% < IAb4 < 10%, 10% ≤ IAb5 < 20%, 20% ≤ IAb6 < 36%, 36% ≤ IAb7 < 45%, 45% ≤ IAb8 < 50%, 50% ≤ IAb9 < 60%, 60% ≤ IAb10 < 80%, 80% ≤ IAb11 < 100%, 100% ≤ IAb12 ≤ 110%.

TABLE IA4
Inhibitory activity of the compounds of the present disclosure, designed based on the
IRAK4 inhibitors, on the Proliferation of Human B-Cell Lymphoma Cells (RPMI-8226)

| | Inhibition | Inhibition | | Inhibition | Inhibition | | Inhibition | Inhibition |
|---|---|---|---|---|---|---|---|---|
| | Rate at | Rate at | | Rate at | Rate at | | Rate at | Rate at |
| | 500 nM | 10 nM | Comp. | 500 nM | 10 nM | Comp. | 500 nM | 10 nM |
| Comp. No. | (%) | (%) | No. | (%) | (%) | No. | (%) | (%) |
| PF06650833 | IAc3 | IAc1 | GT-05285 | IAc6 | IAc7 | GT-05816 | IAc4 | IAc7 |
| GT-05184 | IAc10 | IAc5 | GT-05291 | IAc6 | IAc6 | GT-05818 | IAc3 | IAc5 |
| GT-05188 | IAc8 | IAc7 | GT-05747 | IAc6 | IAc6 | GT-05819 | IAc6 | IAc4 |
| GT-05222 | IAc7 | IAc7 | GT-05748 | IAc4 | IAc7 | GT-05825 | IAc6 | IAc4 |
| GT-05224 | IAc7 | IAc5 | GT-05749 | IAc4 | IAc6 | GT-08653 | IAc6 | IAc4 |
| GT-05236 | IAc4 | IAc5 | GT-05750 | IAc5 | IAc6 | GT-08659 | IAc7 | IAc4 |
| GT-05239 | IAc5 | IAc7 | GT-05752 | IAc6 | IAc4 | GT-08661 | IAc6 | IAc5 |
| GT-05242 | IAc7 | IAc5 | GT-05760 | IAc7 | IAc6 | GT-08667 | IAc7 | IAc5 |
| GT-05318 | IAc9 | IAc5 | GT-05803 | IAc5 | IAc6 | GT-08668 | IAc7 | IAc5 |
| GT-05319 | IAc8 | IAc7 | GT-05806 | IAc6 | IAc5 | GT-08669 | IAc7 | IAc5 |

Note:
in table IA4, the symbols IAc1, IAc2, IAc3, IAc4, IAc5, IAc6, IAc7, IAc8, IAc9, and IAc10 are defined as follows: 0% < IAc1 < 5%, 5% ≤ IAc2 < 10%, 10% ≤ IAc3 < 15%, 15% ≤ IAc4 < 20%, 20% ≤ IAc5 < 25%, 25% ≤ IAc6 < 30%, 30% ≤ IAc7 < 40%, 40% ≤ IAc8 < 60%, 60% ≤ IAc9 < 80%, 80% ≤ IAc10 ≤ 100%.

TABLE IA5
Inhibitory activity of the compounds of the present disclosure, designed based on the
IRAK4 inhibitors, on the Proliferation of Human Multiple myeloma cell line (MM.1S)

| | Inhibition | Inhibition | | Inhibition | Inhibition | | Inhibition | Inhibition |
|---|---|---|---|---|---|---|---|---|
| | Rate at | Rate at | | Rate at | Rate at | | Rate at | Rate at |
| Comp. | 500 nM | 10 nM | Comp. | 500 nM | 10 nM | Comp. | 500 nM | 10 nM |
| No. | (%) | (%) | No. | (%) | (%) | No. | (%) | (%) |
| KT-474 | IAd4 | IAd2 | GT- | IAd9 | IAd4 | GT- | IAd5 | IAd3 |
| | | | 05224 | | | 05291 | | |
| GT- | IAd10 | IAd2 | GT- | IAd9 | IAd3 | GT- | IAd10 | IAd5 |
| 05184 | | | 05318 | | | 05749 | | |
| GT- | IAd7 | IAd3 | GT- | IAd7 | IAd7 | GT- | IAd7 | IAd6 |
| 05188 | | | 05319 | | | 05760 | | |
| GT- | IAd6 | IAd7 | GT- | IAd9 | IAd8 | GT- | IAd6 | IAd7 |
| 05222 | | | 05285 | | | 05803 | | |

Note:
in table IA5, the symbols IAd1, IAd2, IAd3, IAd4, IAd5, IAd6, IAd7, IAd8, IAd9, and IAd10 are defined as follows: 0% < IAd1 < 5%, 5% ≤ IAd2 < 10%, 10% ≤ IAd3 < 15%, 15% ≤ IAd4 < 20%, 20% ≤ IAd5 < 25%, 25% ≤ IAd6 < 30%, 30% ≤ IAd7 < 40%, 40% ≤ IAd8 < 60%, 60% ≤ IAd9 < 80%, 80% ≤ IAd10 ≤ 100%.

TABLE IB1
Inhibitory activity (IC₅₀, nM) of the compounds
of the present disclosure, designed based on the
ALK inhibitors, on tumor cell proliferation

| Comp. No. | SR |
|---|---|
| brigatinib | D |
| alectinib | D |
| GT-06822 | A++ |
| GT-06823 | A++ |
| GT-07298 | A++ |
| GT-07320 | A |
| GT-07321 | A+ |
| GT-07322 | A++ |
| GT-07340 | A++ |
| GT-07341 | A++ |
| GT-07342 | A |
| GT-07343 | A+ |
| GT-07344 | A+ |
| GT-07362 | A++ |
| GT-07363 | A |

Note:
in table IB1, the symbols A++, A+, A, B, C, D, E, and F are defined as follows: 0 nM < A+++ ≤ 10 nM; 10 nM < A++ ≤ 50 nM; 50 nM < A+ ≤ 100 nM; 100 nM < A ≤ 300 nM; 300 nM < B ≤ 500 nM; 500 nM < C ≤ 1000 nM; 1000 nM < D ≤ 5000 nM; 5000 nM < E ≤ 10000 nM; F > 10000 nM. The symbol “—” represents not tested.

TABLE IB2
Inhibitory activity of the compounds of the present disclosure,
designed based on the ALK inhibitors, on tumor cell proliferation

| | MV-4-11 cell | | H2228 cell | | SR cell | |
|---|---|---|---|---|---|---|
| | Inhibition | Inhibition | Inhibition | Inhibition | Inhibition | Inhibition |
| | Rate at 500 | Rate at 10 | Rate at 500 | Rate at 10 | Rate at 500 | Rate at 10 |
| Comp. No. | nM (%) | nM (%) | nM (%) | nM (%) | nM (%) | nM (%) |
| alectinib | IBa6 | IBa2 | IBa5 | IBa2 | IBa10 | IBa4 |
| GT-08251 | IBa9 | IBa6 | — | — | — | — |
| GT-08253 | IBa7 | IBa4 | — | — | — | — |
| GT-08254 | IBa9 | IBa7 | — | — | — | — |
| GT-08148 | IBa9 | IBa2 | IBa7 | IBa4 | IBa11 | IBa6 |
| GT-08146 | IBa11 | IBa4 | IBa9 | IBa5 | IBa11 | IBa7 |
| GT-08147 | IBa9 | IBa5 | IBa7 | IBa5 | IBa11 | IBa3 |

Note:
in table IB2, the symbols IBa1, IBa2, IBa3, IBa4, IBa5, IBa6, IBa7, IBa8, IBa9, IBa10, and IBa11 are defined as follows: 0% < IBa1 < 5%, 5% ≤ IBa2 < 10%, 10% ≤ IBa3 < 15%, 15% ≤ IBa4 < 20%, 20% ≤ IBa5 < 30%, 30% ≤ IBa6 < 45%, 45% ≤ IBa7 < 55%, 55% ≤ IBa8 <60%, 60% ≤ IBa9 < 80%, 80% ≤ IBa10 < 90%, 90% ≤ IBa11 ≤ 100%. The symbol “—” represents not tested.

TABLE IC1
Inhibitory activity (IC₅₀, nM) of the compounds
of the present disclosure, designed based on the
BTK inhibitors, on tumor cell proliferation

| | names | | | | |
|---|---|---|---|---|---|
| Comp. No. | MGC803 | Jurkat | H1975 | MM.1S | JEKO-1 |
| ibrutinib | F | F | D | F | F |
| Orelabrutinib | F | F | F | F | F |
| GT-05075 | A+ | A+ | A+ | A++ | A+ |

Note:
in table IC1, the symbols A++, A+, A, B, C, D, E, and F are defined as follows: 0 nM < A+++ ≤ 10 nM; 10 nM < A++ ≤ 50 nM; 50 nM < A+ ≤ 100 nM; 100 nM < A ≤ 300 nM; 300 nM < B ≤ 500 nM; 500 nM < C ≤ 1000 nM; 1000 nM < D ≤ 5000 nM; 5000 nM < E ≤ 10000 nM; F > 10000 nM. The symbol “—” represents not tested.

TABLE ID1
Inhibitory activity (IC₅₀, nM) of the compounds
of the present disclosure, designed based on the
EGFR inhibitors, on tumor cell proliferation

| | names |
|---|---|
| Comp. No. | H1975 |
| afatinib | B |
| erlotinib | E |
| GT-05196 | A++ |
| GT-05114 | A |
| GT-06159 | A+++ |
| GT-06160 | A |
| GT-06161 | A++ |
| GT-06162 | A++ |
| GT-06172 | A+++ |
| GT-06173 | A+ |
| GT-06174 | A++ |
| GT-06175 | A++ |
| GT-06241 | A++ |
| GT-06242 | A+ |
| GT-06250 | A+++ |
| GT-06251 | A |
| GT-06315 | A+++ |
| GT-06316 | A+ |
| GT-06324 | A++ |

Note:
in table ID1, the symbols A++, A+, A, B, C, D, E, and F are defined as follows: 0 nM < A+++ ≤ 10 nM; 10 nM < A++ ≤ 50 nM; 50 nM < A+ ≤ 100 nM; 100 nM < A ≤ 130 nM; 130 nM < B ≤ 200 nM; 200 nM < C ≤ 500 nM; 500 nM < D ≤ 1000 nM; 1000 nM < E ≤ 5000 nM; 5000 nM < F ≤ 10000 nM; G > 10000 nM. The symbol “—” represents not tested.

TABLE IE1
Inhibitory activity (IC₅₀, nM) of the compounds
of the present disclosure, designed based on the
CDK4/6 inhibitors, on tumor cell proliferation

| | names | | | | |
|---|---|---|---|---|---|
| Comp. No. | CCRF-CEM | MCF-7 | Molt-4 | MV-4-11 | SR |
| Ribociclib | E | E | G | F | F |
| Palbociclib | E | E | E | B | E |
| GT-06663 | — | | — | A++ | C |
| GT-06672 | C | A | — | A++ | A |
| GT-06733 | A | — | A | A++ | A |
| GT-06734 | C | — | C | A+ | A |
| GT-06736 | A | A+ | A | A++ | A++ |
| GT-06737 | — | — | I | B | A |
| GT-06837 | A | A | A | A++ | A++ |

Note:
in table IE1, the symbols A++, A+, A, B, C, D, E, and F are defined as follows: 0 nM < A+++ ≤ 10 nM; 10 nM < A++ ≤ 50 nM; 50 nM < A+ ≤ 100 nM; 100 nM < A ≤ 300 nM; 300 nM < B ≤ 400 nM; 400 nM < C ≤ 800 nM; 800 nM < D ≤ 1000 nM; 1000 nM < E ≤ 5000 nM; 5000 nM < F ≤ 10000 nM; G > 10000 nM. The symbol “—” represents not tested.

TABLE IE2
Inhibitory activity of the compounds of the present disclosure,
designed based on the CDK4/6 inhibitors, on the proliferation
of Human lung cancer cell line (H292)

| | Inhibition | |
|---|---|---|
| | Rate at 500 | Inhibition Rate |
| Comp. No. | nM (%) | at 10 nM (%) |
| Palbociclib | IEa6 | IEa3 |
| GT-05199 | IEa8 | IEa9 |
| GT-05202 | IEa8 | IEa8 |
| GT-05205 | IEa8 | IEa8 |
| GT-05812 | IEa7 | IEa8 |
| GT-06335 | IEa7 | IEa8 |
| GT-06338 | IEa9 | IEa7 |
| GT-06487 | IEa9 | IEa6 |
| GT-06489 | IEa9 | IEa7 |
| GT-06573 | IEa9 | IEa7 |
| GT-06583 | IEa9 | IEa8 |

Note:
in table IE2, the symbols IEa1, IEa2, IEa3, IEa4, IEa5, IEa6, IEa7, IEa8, IEa9, IEa10, and IEa11 are defined as follows: 0 < IEa1 < 5%, 5% ≤ IEa2 < 10%, 10% ≤ IEa3 < 15%, 15% ≤ IEa4 < 20%, 20% ≤ IEa5 < 25%, 25% ≤ IEa6 < 30%, 30% ≤ IEa7 < 35%, 35% ≤ IEa8 < 40%,40% ≤ IEa9 < 60%, 60% ≤ IEa10 < 80%,80% ≤ IEa11 ≤ 100%.

TABLE IE3
Inhibitory activity of the compounds of the present disclosure,
designed based on the CDK4/6 inhibitors, on the proliferation
of Human myeloid monocytic leukemia cell line (MV-4-11)

| | Inhibition | |
|---|---|---|
| | Rate at 500 | Inhibition Rate |
| Comp. No. | nM (%) | at 10 nM (%) |
| Ribociclib | IEb7 | IEb2 |
| GT-05202 | IEb9 | IEb4 |
| GT-05205 | IEb9 | IEb5 |
| GT-05812 | IEb9 | IEb4 |
| GT-06335 | IEb9 | IEb3 |
| GT-06489 | IEb9 | IEb6 |
| GT-06573 | IEb10 | IEb9 |
| GT-06583 | IEb9 | IEb9 |
| GT-06586 | IEb9 | IEb6 |

Note:
in table IE3, the symbols IEb1, IEb2, IEb3, IEb4, IEb5, IEb6, IEb7, IEb8, IEb9, IEb10, and IEb11 are defined as follows: 0 < IEb1 < 5%, 5% ≤ IEb2 < 10%, 10% ≤ IEb3 < 15%, 15% ≤ IEb4 < 20%, 20% ≤ IEb5 < 25%, 25% ≤ IEb6 < 30%, 30% ≤ IEb7 < 35%, 35% ≤ IEb8 < 40%, 40% ≤ IEb9 < 60%, 60% ≤ IEb10 < 80%, 80% ≤ IEb11 ≤ 100%.

TABLE IF1
Inhibitory activity of the compounds of the present disclosure, designed based on the AR
inhibitors, on the proliferation of Human diffuse large B-cell lymphoma cell line (TMD8)

| | Inhibition | Inhibition | | Inhibition | Inhibition | | Inhibition | Inhibition |
|---|---|---|---|---|---|---|---|---|
| | Rate at | Rate at | | Rate at | Rate at | | Rate at | Rate at |
| | 500 nM | 10 nM | Comp. | 500 nM | 10 nM | Comp. | 500 nM | 10 nM |
| Comp. No. | (%) | (%) | No. | (%) | (%) | No. | (%) | (%) |
| Enzalutamide | IFa3 | IFa3 | GT- | IFa8 | IFa4 | GT- | IFa9 | IFa4 |
| | | | 05346 | | | 05824 | | |
| GT-05226 | IFa6 | IFa3 | GT- | IFa5 | IFa2 | GT- | IFa9 | IFa6 |
| | | | 05347 | | | 05826 | | |
| GT-05227 | IFa6 | IFa4 | GT- | IFa9 | IFa6 | GT- | IFa8 | IFa3 |
| | | | 05301 | | | 05829 | | |
| GT-05229 | IFa10 | IFa9 | GT- | IFa9 | IFa7 | GT- | IFa6 | IFa3 |
| | | | 05304 | | | 05830 | | |
| GT-05230 | IFa5 | IFa2 | GT- | IFa9 | IFa6 | GT- | IFa9 | IFa3 |
| | | | 05305 | | | 05832 | | |
| GT-05231 | IFa9 | IFa2 | GT- | IFa5 | IFa3 | GT- | IFa9 | IFa4 |
| | | | 05306 | | | 05834 | | |
| GT-05232 | IFa6 | IFa3 | GT- | IFa9 | IFa4 | GT- | IFa7 | IFa4 |
| | | | 05821 | | | 06478 | | |
| GT-05235 | IFa8 | IFa2 | GT- | IFa8 | IFa3 | | | |
| | | | 05823 | | | | | |

Note:
in table IF1, the symbols IFa1, IFa2, IFa3, IFa4, IFa5, IFa6, IFa7, IFa8, IFa9, IFa10, and IFa11 are defined as follows: 0 < IFa1 < 5%, 5% ≤ IFa2 < 10%, 10% ≤ IFa3 < 15%, 15% ≤ IFa4 < 20%, 20% ≤ IFa5 < 25%, 25% ≤ IFa6 < 30%, 30% ≤ IFa7 < 35%, 35% ≤ IFa8 < 40%, 40% ≤ IFa9 < 60%, 60% ≤ IFa10 < 80%, 80% ≤ IFa11 ≤ 100%.

TABLE IF2
Inhibitory activity of the compounds of the present disclosure, designed based on the AR inhibitors,
on the proliferation of Human acute lymphocytic leukemia T lymphocytes cell line (CCRF-CEM)

| | Inhibition | Inhibition | | Inhibition | Inhibition | | Inhibition | Inhibition |
|---|---|---|---|---|---|---|---|---|
| | Rate at | Rate at | | Rate at | Rate at | | Rate at | Rate at |
| | 500 nM | 10 nM | Comp. | 500 nM | 10 nM | Comp. | 500 nM | 10 nM |
| Comp. No. | (%) | (%) | No. | (%) | (%) | No. | (%) | (%) |
| Enzalutamide | IFb2 | IFb2 | GT- | IFb6 | IFb5 | GT- | IFb7 | IFb4 |
| | | | 05346 | | | 05824 | | |
| GT-05227 | IFb6 | IFb6 | GT- | IFb7 | IFb5 | GT- | IFb8 | IFb6 |
| | | | 05301 | | | 05826 | | |
| GT-05229 | IFb9 | IFb7 | GT- | IFb7 | IFb6 | GT- | IFb6 | IFb5 |
| | | | 05304 | | | 05829 | | |
| GT-05230 | IFb6 | IFb4 | GT- | IFb8 | IFb6 | GT- | IFb6 | IFb3 |
| | | | 05305 | | | 05830 | | |
| GT-05231 | IFb7 | IFb3 | GT- | IFb6 | IFb5 | GT- | IFb7 | IFb1 |
| | | | 05306 | | | 05832 | | |
| GT-05233 | IFb6 | IFb5 | GT- | IFb7 | IFb5 | GT- | IFb8 | IFb4 |
| | | | 05821 | | | 05834 | | |
| GT-05235 | IFb7 | IFb4 | GT- | IFb6 | IFb3 | | | |
| | | | 05823 | | | | | |

Note:
in table IF2, the symbols IFb1, IFb2, IFb3, IFb4, IFb5, IFb6, IFb7, IFb8, IFb9, and IFb10 are defined as follows: −5% ≤ IFb1 ≤ 0%, 0% < IFb2 < 5%, 5% ≤ IFb3 < 10%, 10% ≤ IFb4 < 15%, 15% ≤ IFb5 < 20%, 20% ≤ IFb6 < 30%, 30% ≤ IFb7 < 40%, 40% ≤ IFb8 < 60%, 60% ≤ IFb9 < 80%, 80% ≤ IFb10 ≤ 100%.

TABLE IF3
Inhibitory activity of the compounds of the present disclosure, designed based on the
AR inhibitors, on the proliferation of Human T lymphocytic leukemia cell line (Jurkat)

| | Inhibition | Inhibition | | Inhibition | Inhibition | | Inhibition | Inhibition |
|---|---|---|---|---|---|---|---|---|
| | Rate at | Rate at | | Rate at | Rate at | | Rate at | Rate at |
| Comp. | 500 nM | 10 nM | Comp. | 500 nM | 10 nM | Comp. | 500 nM | 10 nM |
| No. | (%) | (%) | No. | (%) | (%) | No. | (%) | (%) |
| Enzalutamide | IFc2 | IFc2 | GT-05346 | IFc5 | IFc1 | GT-05824 | IFc6 | IFc2 |
| GT-05227 | IFc5 | IFc2 | GT-05301 | IFc5 | IFc3 | GT-05826 | IFc7 | IFc5 |
| GT-05229 | IFc7 | IFc5 | GT-05305 | IFc7 | IFc3 | GT-05830 | IFc5 | IFc3 |
| GT-05230 | IFc5 | IFc3 | GT-05821 | IFc5 | IFc3 | GT-05832 | IFc5 | IFc1 |
| GT-05231 | IFc6 | IFc3 | GT-05823 | IFc5 | IFc2 | GT-05834 | IFc7 | IFc4 |
| GT-05235 | IFc6 | IFc2 | | | | | | |

Note:
in table IF3, the symbols IFc1, IFc2, IFc3, IFc4, IFc5, IFc6, IFc7, IFc8, and IFc9 are defined as follows: 0% < IFc1 < 5%, 5% ≤ IFc2 < 10%, 10% ≤ IFc3 < 15%, 15% ≤ IFc4 < 20%, 20% ≤ IFc5 < 30%, 30% ≤ IFc6 < 40%, 40% ≤ IFc7 < 60%, 60% ≤ IFc8 < 80%, 80% ≤ IFc9 ≤ 100%.

TABLE IG1
Inhibitory activity of the compounds of the present disclosure,
designed based on the FAK inhibitors, on the proliferation
of Human Multiple myeloma cell line (MM.1S)

| | | Inhibition |
|---|---|---|
| | Inhibition Rate | Rate at 10 |
| Comp. No. | at 500 nM (%) | nM (%) |
| Defactinib | IGa2 | IGa2 |
| PND-1186 | IGa5 | IGa2 |
| GT-07468 | IGa9 | IGa5 |
| GT-07647 | IGa7 | IGa5 |
| GT-07660 | IGa8 | IGa4 |
| GT-07661 | IGa7 | IGa6 |
| GT-07663 | IGa7 | IGa2 |
| GT-07664 | IGa7 | IGa5 |
| GT-08245 | IGa9 | IGa2 |
| GT-08246 | IGa6 | IGa6 |
| GT-08248 | IGa8 | IGa6 |

Note:
in table IG1, the symbols IGa1, IGa2, IGa3, IGa4, IGa5, IGa6, IGa7, IGa8, and IGa9 are defined as follows: 0% < IGa1 < 5%, 5% ≤ IGa2 < 10%, 10% ≤ IGa3 < 15%, 15% ≤ IGa4 < 20%, 20% ≤ IGa5 < 30%, 30% ≤ IGa6 < 40%, 40% ≤ IGa7 < 60%, 60% ≤ IGa8 < 80%, 80% ≤ IGa9 ≤ 100%.

TABLE IG2
Inhibitory activity of the compounds of the present disclosure,
designed based on the FAK inhibitors, on the proliferation
of Human myeloid monocytic leukemia cell line (MV-4-11)

| | Inhibition | Inhibition |
|---|---|---|
| | Rate at 500 | Rate at 10 |
| Comp. No. | nM (%) | nM (%) |
| Defactinib | IGb10 | IGb3 |
| PND-1186 | IGb10 | IGb3 |
| GT-07504 | IGb12 | IGb1 |
| GT-07468 | IGb12 | IGb7 |
| GT-07469 | IGb12 | IGb2 |
| GT-07514 | IGb12 | IGb7 |
| GT-07647 | IGb12 | IGb8 |
| GT-07660 | IGb12 | IGb9 |
| GT-08245 | IGb12 | IGb6 |
| GT-08248 | IGb12 | IGb6 |
| GT-08136 | IGb12 | IGb1 |
| GT-08138 | IGb12 | IGb6 |
| GT-08142 | IGb12 | IGb8 |
| GT-08144 | IGb12 | IGb7 |

Note:
in table IG2, the symbols IGb1, IGb2, IGb3, IGb4, IGb5, IGb6, IGb7, IGb8, IGb9, IGb10, IGb11, and IGb12 are defined as follows: - 5% ≤ IGb1 ≤ 0%, 0% < IGb2 < 5%, 5% ≤ IGb3 < 10%, 10% ≤ IGb4 < 15%, 15% ≤ IGb5 < 20%, 20% ≤ IGb6 < 30%, 30% ≤ IGb7 < 40%,40% ≤ IGb8 < 50%, 50% ≤ IGb9 < 60%,60% ≤ IGb10 < 70%, 70% ≤ IGb11 < 80%, 80% ≤ IGb12 ≤ 100%.

TABLE IG3
Inhibitory activity of the compounds of the present disclosure,
designed based on the FAK inhibitors, on the proliferation
of Human breast cancer cell line (MDA-MB-231)

| | Inhibition | Inhibition |
|---|---|---|
| | Rate at 500 | Rate at 10 |
| Comp. No. | nM (%) | nM (%) |
| Defactinib | IGc6 | IGc6 |
| PND-1186 | IGc7 | IGc6 |
| GT-07468 | IGc8 | IGc5 |
| GT-07469 | IGc8 | IGc6 |
| GT-07647 | IGc10 | IGc7 |
| GT-08245 | IGc11 | IGc6 |
| GT-08248 | IGc10 | IGc5 |
| GT-08136 | IGc8 | IGc5 |

Note:
in table IG3, the symbols IGc1, IGc2, IGc3, IGc4, IGc5, IGc6, IGc7, IGc8, IGc9, IGc10, and IGc11 are defined as follows: 0% < IGc1 < 5%, 5% ≤ IGc2 < 10%, 10% ≤ IGc3 < 15%, 15% ≤ IGc4 < 20%, 20% ≤ IGc5 < 30%, 30% ≤ IGc6 < 40%, 40% ≤ IGc7 < 50%, 50% ≤ IGc8 < 60%, 60% ≤ IGc9 < 70%, 70% ≤ IGc10 < 80%, 80% ≤ IGc11 ≤ 100%.

TABLE IH1
Inhibitory activity of the compounds of the present disclosure,
designed based on the BCL-2inhibitors, on the proliferation
of Human prostate cancer cell line (NCI-H358)

| | Inhibition | Inhibition |
|---|---|---|
| | Rate at 500 | Rate at 10 |
| Comp. No. | nM (%) | nM (%) |
| ABT-199 | IHa2 | IHa2 |
| GT-07811 | IHa7 | IHa4 |
| GT-07812 | IHa8 | IHa6 |
| GT-07813 | IHa5 | IHa4 |
| GT-07814 | IHa7 | IHa4 |
| GT-07815 | IHa6 | IHa4 |
| GT-07821 | IHa5 | IHa3 |

Note:in table IH1, the symbols IHa1, IHa2, IHa3, IHa4, IHa5, IHa6, IHa7, IHa8, IHa9, IHa10, and IHa11 are defined as follows: 0% < IHa1 < 5%, 5% ≤ IHa2 < 10%, 10% ≤ IHa3 < 15%, 15% ≤ IHa4 < 20%, 20% ≤ IHa5 < 30%, 30% ≤ IHa6 < 40%, 40% ≤ IHa7 < 50%, 50% ≤ IHa8 < 60%, 60% ≤ IHa9 < 70%, 70% ≤ IHa10 < 80%, 80% ≤ IHa11 ≤ 100%.

TABLE IH2
Inhibitory activity of the compounds of the present disclosure, designed
based on the BCL-2 inhibitors, on the proliferation of Human acute
lymphocytic leukemia T lymphocytes cell line (CCRF-CEM)

| | Inhibition | Inhibition |
|---|---|---|
| | Rate at 500 | Rate at 10 |
| Comp. No. | nM (%) | nM (%) |
| ABT-199 | IHb1 | IHb1 |
| GT-07811 | IHb12 | IHb3 |
| GT-07812 | IHb12 | IHb6 |
| GT-07813 | IHb10 | IHb3 |
| GT-07814 | IHb12 | IHb9 |
| GT-07815 | IHb9 | IHb1 |
| GT-07816 | IHb8 | IHb3 |
| GT-07821 | IHb11 | IHb5 |

Note:
in table IH2, the symbols IHb1, IHb2, IHb3, IHb4, IHb5, IHb6, IHb7, IHb8, IHb9, IHb10, IHb11, and IHb12 are defined as follows: - 5% ≤ IHb1 < 0%, 0% < IHb2 < 5%, 5% ≤ IHb3 < 10%, 10% ≤ IHb4 < 15%, 15% ≤ IHb5 < 20%, 20% ≤ IHb6 < 30%, 30% ≤ IHb7 < 40%,40% ≤ IHb8 < 50%, 50% ≤ IHb9 < 60%, 60% ≤ IHb10 < 70%, 70% ≤ IHb11 < 80%, 80% ≤ IHb12 ≤ 100%.

TABLE IH3
Inhibitory activity of the compounds of the present disclosure,
designed based on the BCL-2 inhibitors, on the proliferation
of Human acute T lymphoblastic leukemia cell line (Molt-4)

| | Inhibition | Inhibition |
|---|---|---|
| | Rate at 500 | Rate at 10 |
| Comp. No. | nM (%) | nM (%) |
| ABT-199 | IHc7 | IHc1 |
| GT-07811 | IHc12 | IHc8 |
| GT-07812 | IHc12 | IHc9 |
| GT-07813 | IHc12 | IHc4 |
| GT-07814 | IHc12 | IHc12 |
| GT-07815 | IHc12 | IHc3 |
| GT-07816 | IHc10 | IHc4 |
| GT-07821 | IHc12 | IHc2 |

Note:
in table IH3, the symbols IHb1, IHb2, IHb3, IHb4, IHb5, IHb6, IHb7, IHb8, IHb9, IHb10, IHb11, and IHb12 are defined as follows: - 5% ≤ IHc1 ≤ 0%, 0% < IHc2 < 5%, 5% ≤ IHc3 < 10%, 10% ≤ IHc4 < 15%, 15% ≤ IHc5 < 20%, 20% ≤ IHc6 < 30%, 30% ≤ IHc7 < 40%, 40% ≤ IHc8 < 50%, 50% ≤ IHc9 < 60%, 60% ≤ IHc10 < 70%, 70% ≤ IHc11 < 80%, 80% ≤ IHc12 ≤ 100%.

TABLE III
Inhibitory activity of the compounds of the present disclosure, designed based on the EZH2
inhibitors, on the proliferation of Human malignant melanoma cell line (A375)

| | Inhibition | Inhibition | | Inhibition | Inhibition |
|---|---|---|---|---|---|
| | Rate at 500 | Rate at 10 | | Rate at 500 | Rate at 10 |
| Comp. No. | nM (%) | nM (%) | Comp. No. | nM (%) | nM (%) |
| Tazemetostat | IIal | IIal | GT-08515 | IIa5 | IIa5 |
| GT-08421 | IIa5 | IIa4 | GT-08516 | IIa5 | IIa5 |
| GT-08423 | IIa6 | IIa5 | GT-08518 | IIa5 | IIa5 |
| GT-08508 | IIa5 | IIa5 | | | |

Note:
in table III, the symbols IIal, IIa2, IIa3, IIa4, IIa5, IIa6, IIa7, IIa8, IIa9, IIa10, and IIa11 are defined as follows: 0% < IIal < 5%, 5% ≤ IIa2 < 10%, 10% ≤ IIa3 < 15%, 15% ≤ IIa4 < 20%, 20% ≤ IIa5 < 30%, 30% ≤ IIa6 < 40%, 40% ≤ IIa7 < 50%, 50% ≤ IIa8 < 60%, 60% ≤ IIa9 < 70%, 70% ≤ IIa10 < 80%, 80% ≤ IIal1 ≤ 100%.

TABLE II2
Inhibitory activity of the compounds of the present disclosure, designed based on the EZH2
inhibitors, on the proliferation of Human neuroblastoma cell line (SK-N-AS)

| | Inhibition | Inhibition | | Inhibition | Inhibition |
|---|---|---|---|---|---|
| | Rate at 500 | Rate at 10 | | Rate at 500 | Rate at 10 |
| Comp. No. | nM (%) | nM (%) | Comp. No. | nM (%) | nM (%) |
| UNC1999 | IIb1 | IIb1 | GT-08512 | IIb5 | IIb3 |
| GT-08421 | IIb5 | IIb5 | GT-08515 | IIb5 | IIb5 |
| GT-08422 | IIb5 | IIb2 | GT-08519 | IIb5 | IIb3 |
| GT-08423 | IIb7 | IIb5 | GT-08521 | IIb6 | IIb5 |
| GT-08510 | IIb5 | IIb4 | GT-08524 | IIb5 | IIb4 |

Note:
in table II2, the symbols IIb1, IIb2, IIb3, IIb4, IIb5, IIb6, IIb7, IIb8, IIb9, IIb10, and IIb11 are defined as follows: 0% < IIb1 < 5%, 5% ≤ IIb2 < 10%, 10% ≤ IIb3 < 15%, 15% ≤ IIb4 < 20%, 20% ≤ IIb5 < 30%, 30% ≤ IIb6 < 40%, 40% ≤ IIb7 < 50%, 50% ≤ IIb8 < 60%, 60% ≤ IIb9 < 70%, 70% ≤ IIb10 < 80%, 80% ≤ IIb11 ≤ 100%.

TABLE IJ1
Inhibitory activity of the compounds of the present disclosure, designed based on the Tau
inhibitors, on the proliferation of Human Multiple myeloma cell line (MM.1S)

| | Inhibition | Inhibition | | Inhibition | Inhibition |
|---|---|---|---|---|---|
| | Rate at 500 | Rate at 10 | | Rate at 500 | Rate at 10 |
| Comp. No. | nM (%) | nM (%) | Comp. No. | nM (%) | nM (%) |
| GT-08557 | IJa8 | IJa6 | GT-08555 | IJa9 | IJa6 |
| GT-08544 | IJa7 | IJa6 | GT-08563 | IJa9 | IJa4 |
| GT-08539 | IJa9 | IJa5 | | | |

Note:
in table IJ1, the symbols IJal, IJa2, IJa3, IJa4, IJa5, IJa6, IJa7, IJa8, IJa9, IJa10, and IJa11 are defined as follows: 0% < IJal < 5%, 5% ≤ IJa2 < 10%, 10% ≤ IJa3 <15%, 15% ≤ IJa4 <20%, 20% ≤IJa5 <30%, 30% ≤ IJa6 < 40%, 40% ≤ IJa7 < 50%, 50% ≤ IJa8 < 60%, 60% ≤ IJa9 < 70%, 70% ≤ IJa10 < 80%, 80% ≤ IJa11 ≤ 100%.

TABLE IJ2
Inhibitory activity of the compounds of the present disclosure, designed based on the Tau
inhibitors, on the proliferation of Human myeloid monocytic leukemia cell line (MV-4-11)

| | Inhibition | Inhibition | | Inhibition | Inhibition |
|---|---|---|---|---|---|
| | Rate at 500 | Rate at 10 | | Rate at 500 | Rate at 10 |
| Comp. No. | nM (%) | nM (%) | Comp. No. | nM (%) | nM (%) |
| GT-08558 | IJb9 | IJb1 | GT-08555 | IJb11 | IJb7 |
| GT-08539 | IJb9 | IJb7 | GT-08563 | IJb12 | IJb5 |
| GT-08561 | IJb9 | IJb2 | GT-08564 | IJb11 | IJb2 |

Note:
in table IJ2, the symbols IJb1, IJb2, IJb3, IJb4, IJb5, IJb6, IJb7, IJb8, IJb9, IJb10, IJb11, and IJb12 are defined as follows: −5% ≤ IJb1 ≤ 0%, 0% < IJb2 < 5%, 5% ≤ IJb3 < 10%, 10% ≤ IJb4 < 15%, 15% ≤ IJb5 < 20%, 20% ≤ IJb6 < 30%, 30% ≤ IJb7 < 40%, 40% ≤ IJb8 < 50%, 50% ≤ IJb9 < 60%, 60% ≤ IJb10 < 70%, 70% ≤ IJb11 < 80%, 80% ≤ IJb12 ≤ 100%.

TABLE IJ3
Inhibitory activity of the compounds of the present disclosure, designed based on the Tau
inhibitors, on the proliferation of Human diffuse large B-cell lymphoma cell line (TMD8)

| | Inhibition | Inhibition | | Inhibition | Inhibition |
|---|---|---|---|---|---|
| | Rate at 500 | Rate at 10 | | Rate at 500 | Rate at 10 |
| Comp. No. | nM (%) | nM (%) | Comp. No. | nM (%) | nM (%) |
| GT-08557 | IJc9 | IJc1 | GT-08553 | IJc7 | IJc3 |
| GT-08558 | IJc11 | IJc2 | GT-08563 | IJc11 | IJc4 |
| GT-08539 | IJc9 | IJc5 | GT-08564 | IJc11 | IJc3 |

Note:
in table IJ3, the symbols IJc1, IJc2, IJc3, IJc4, IJc5, IJc6, IJc7, IJc8, IJc9, IJc10, and IJe11 are defined as follows: 0% < IJcl < 5%, 5% ≤ IJc2 < 10%, 10% ≤ IJc3 < 15%, 15% ≤ IJc4 < 20%, 20% ≤ IJc5 < 30%, 30% ≤ IJc6 < 40%, 40% ≤ IJc7 < 50%, 50% ≤ IJc8 < 60%, 60% ≤ IJc9 < 70%, 70% ≤ IJc10 < 80%, 80% ≤ IJc11 ≤ 100%.

TABLE IK1
Inhibitory activity of the compounds of the present disclosure, designed based on the AKT
inhibitors, on the proliferation of Human neuroblastoma cell line (SK-N-AS)

| | Inhibition | Inhibition | | Inhibition | Inhibition |
|---|---|---|---|---|---|
| | Rate at 500 | Rate at 10 | | Rate at 500 | Rate at 10 |
| Comp. No. | nM (%) | nM (%) | Comp. No. | nM (%) | nM (%) |
| GT-08574 | IKa5 | IKa2 | GT-08576 | IKa5 | IKa4 |

Note:
in table IK1, the symbols IKal, IKa2, IKa3, IKa4, IKa5, IKa6, IKa7, IKa8, IKa9, IKal0, and IKa11 are defined as follows: 0% < IKal < 5%, 5% ≤ IKa2 < 10%, 10% ≤ IKa3 < 15%, 15% ≤ IKa4 < 20%, 20% ≤ IKa5 < 30%, 30% ≤ IKa6 < 40%, 40% ≤ IKa7 < 50%, 50% ≤ IKa8 < 60%, 60% ≤ IKa9 < 70%, 70% ≤ IKa10 < 80%, 80% ≤ IKa11 ≤ 100%.

TABLE IL1
Inhibitory activity of the compounds of the present disclosure, designed based on the BCR-ABL
inhibitors, on the proliferation of Multiple myeloma cell line (MM.1S)

| | Inhibition | Inhibition | | Inhibition | Inhibition |
|---|---|---|---|---|---|
| | Rate at 500 | Rate at 10 | | Rate at 500 | Rate at 10 |
| Comp. No. | nM (%) | nM (%) | Comp. No. | nM (%) | nM (%) |
| Dasatinib | ILa4 | ILal | GT-08356 | ILa6 | ILa5 |
| GT-08216 | ILa11 | ILa5 | GT-08359 | ILa6 | ILa3 |
| GT-08227 | ILa5 | ILa5 | GT-08360 | ILa5 | ILa2 |
| GT-08354 | ILa5 | ILa4 | | | |

Note:
in table IL1, the symbols ILal, ILa2, ILa3, ILa4, ILa5, ILa6, ILa7, ILa8, ILa9, ILa10, and ILa11 are defined as follows: 0% < ILal < 5%, 5% ≤ ILa2 < 10%, 10% ≤ ILa3 < 15%, 15% ≤ ILa4 < 20%, 20% ≤ ILa5 < 30%, 30% ≤ ILa6 < 40%, 40% ≤ ILa7 < 50%, 50% ≤ ILa8 < 60%, 60% ≤ ILa9 < 70%, 70% ≤ ILa10 < 80%, 80% ≤ ILa11 ≤ 100%.

TABLE IL2
Inhibitory activity of the compounds of the present disclosure, designed based on the BCR-
ABL inhibitors, on the proliferation of Human myeloid monocytic leukemia cell line (MV-4-11)

| | Inhibition | Inhibition | | Inhibition | Inhibition |
|---|---|---|---|---|---|
| | Rate at 500 | Rate at 10 | | Rate at 500 | Rate at 10 |
| Comp. No. | nM (%) | nM (%) | Comp. No. | nM (%) | nM (%) |
| Dasatinib | ILb8 | ILb4 | GT-08220 | ILb12 | ILb1 |
| GT-08216 | ILb12 | ILb12 | GT-08221 | ILb9 | ILb1 |
| GT-08217 | ILb12 | ILb4 | GT-08352 | ILb12 | ILb7 |
| GT-08218 | ILb12 | ILb3 | GT-08353 | ILb12 | ILb9 |

Note:
in table IL2, the symbols ILb1, ILb2, ILb3, ILb4, ILb5, ILb6, ILb7, ILb8, ILb9, ILb10, ILb11, and ILb12 are defined as follows: −5% ≤ ILb1 ≤ 0%, 0% < ILb2 < 5%, 5% ≤ ILb3 < 10%, 10% ≤ ILb4 < 15%, 15% ≤ ILb5 < 20%, 20% ≤ ILb6 < 30%, 30% ≤ ILb7 < 40%, 40% ≤ ILb8 < 50%, 50% ≤ ILb9 < 60%, 60% ≤ ILb10 < 70%, 70% ≤ ILb11 < 80%, 80% ≤ ILb12 ≤ 100%.

TABLE IL3
Inhibitory activity of the compounds of the present disclosure, designed based on the BCR-
ABL inhibitors, on the proliferation of Human acute lymphocytic leukemia T lymphocytes
cell line (CCRF-CEM)

| | Inhibition | Inhibition | | Inhibition | Inhibition |
|---|---|---|---|---|---|
| | Rate at 500 | Rate at 10 | | Rate at 500 | Rate at 10 |
| Comp. No. | nM (%) | nM (%) | Comp. No. | nM (%) | nM (%) |
| Dasatinib | ILc4 | ILc3 | GT-08352 | ILc5 | ILc4 |
| GT-08216 | ILc11 | ILc5 | GT-08359 | ILc5 | ILc4 |
| GT-08225 | ILc6 | ILc3 | GT-08360 | ILc6 | ILc5 |
| GT-08226 | ILc6 | ILc2 | GT-08361 | ILc5 | ILc5 |

Note:
in table IL3, the symbols ILc1, ILc2, ILc3, ILc4, ILc5, ILc6, ILc7, ILc8, ILc9, ILc10, and ILc11 are defined as follows: 0% < ILcl < 5%, 5% ≤ ILc2 < 10%, 10% ≤ ILc3 < 15%, 15% ≤ ILc4 < 20%, 20% ≤ ILc5 < 30%, 30% ≤ ILc6 < 40%, 40% ≤ ILc7 < 50%, 50% ≤ ILc8 < 60%, 60% ≤ ILc9 < 70%, 70% ≤ ILc10 < 80%, 80% ≤ ILc11 ≤ 100%.

TABLE IM1
Inhibitory activity of the compounds of the present disclosure, designed based on the CDK9
inhibitors, on the proliferation of Human lung cancer cell line (H292)

| | Inhibition | Inhibition | | Inhibition | Inhibition |
|---|---|---|---|---|---|
| | Rate at 500 | Rate at 10 | | Rate at 500 | Rate at 10 |
| Comp. No. | nM (%) | nM (%) | Comp. No. | nM (%) | nM (%) |
| AT7519 | ILb3 | ILb1 | GT-08349 | ILb6 | ILb5 |
| GT-04719 | ILb5 | ILb3 | GT-08350 | ILb8 | ILb8 |
| GT-08346 | ILb9 | ILb2 | GT-08212 | ILb7 | ILb6 |
| GT-08347 | ILb6 | ILb3 | GT-08215 | ILb6 | ILb6 |
| GT-08348 | ILb7 | ILb1 | GT-08229 | ILb7 | ILb6 |

Note:
in table IM1, the symbols IMal, IMa2, IMa3, IMa4, IMa5, IMa6, IMa7, IMa8, IMa9, IMa10, IMa11, and IMa12 are defined as follows: −5% ≤ IMal ≤ 0%, 0% < IMa2 < 5%, 5% ≤ IMa3 < 10%, 10% ≤ IMa4 < 15%, 15% ≤ IMa5 < 20%, 20% ≤ IMa6 < 30%, 30% ≤ IMa7 < 40%, 40% ≤ IMa8 < 50%, 50% ≤ IMa9 < 60%, 60% ≤ IMa10 < 70%, 70% ≤ IMal1 < 80%, 80% ≤ IMa12 ≤ 100%.

TABLE IM2
Inhibitory activity of the compounds of the present disclosure, designed based on the CDK9
inhibitors, on the proliferation of Human B-Cell Lymphoma Cells (RPMI-8226)

| | Inhibition | Inhibition | | Inhibition | Inhibition |
|---|---|---|---|---|---|
| | Rate at 500 | Rate at 10 | | Rate at 500 | Rate at 10 |
| Comp. No. | nM (%) | nM (%) | Comp. No. | nM (%) | nM (%) |
| AT7519 | IMb1 | IMb4 | GT-08350 | IMb7 | IMb6 |
| GT-04719 | IMb7 | IMb6 | GT-08212 | IMb5 | IMb7 |
| GT-08345 | IMb5 | IMb7 | GT-08215 | IMb7 | IMb7 |
| GT-08347 | IMb6 | IMb7 | GT-08229 | IMb6 | IMb6 |

Note:
in table IM2, the symbols IMb1, IMb2, IMb3, IMb4, IMb5, IMb6, IMb7, IMb8, IMb9, IMb10, and IMb11 are defined as follows: −20% ≤ IMb1 < −15%, −15% < IMb2 < −5%, −5% ≤ IMb3 ≤ 0%, 0% < IMb4 < 5%, 5% ≤ IMb5 < 10%, 10% ≤ IMb6 < 20%, 20% ≤ IMb7 < 30%, 30% ≤ IMb8 < 50%, 50% ≤ IMb9 < 70%, 70% ≤ IMb10 < 80%, 80% ≤ IMb11 ≤ 100%.

TABLE IN1
Inhibitory activity of the compounds of the present disclosure, designed based on the RSK
inhibitors, on the proliferation of Human myeloid monocytic leukemia cell line (MV-4-11)

| | Inhibition | Inhibition | | Inhibition | Inhibition |
|---|---|---|---|---|---|
| | Rate at 500 | Rate at 10 | | Rate at 500 | Rate at 10 |
| Comp. No. | nM (%) | nM (%) | Comp. No. | nM (%) | nM (%) |
| BI-D1870 | INa7 | INa3 | GT-08149 | INa10 | INa5 |
| GT-08255 | INa9 | INa2 | GT-08151 | INa11 | INa5 |
| GT-08256 | INa11 | INa7 | | | |

Note:
in table IN1, the symbols INal, INa2, INa3, INa4, INa5, INa6, INa7, INa8, INa9, INa10, and INa11 are defined as follows: −20% ≤ INal < −15%, −15% < INa2 < −5%,−5% ≤ INa3 ≤ 0%,0% < INa4 < 5%, 5% ≤ INa5 < 10%, 10% ≤ INa6 < 20%, 20% ≤ INa7 < 30%, 30% ≤ INa8 < 50%, 50% ≤ INa9 < 70%,70% ≤ INa10 < 80%, 80% ≤ INa11 ≤ 100%.

TABLE IN2
Inhibitory activity of the compounds of the present disclosure, designed based on the RSK
inhibitors, on the proliferation of Human acute T lymphoblastic leukemia cell line (Molt-4)

| | Inhibition | Inhibition | | Inhibition | Inhibition |
|---|---|---|---|---|---|
| | Rate at 500 | Rate at 10 | | Rate at 500 | Rate at 10 |
| Comp. No. | nM (%) | nM (%) | Comp. No. | nM (%) | nM (%) |
| BI-D1870 | INb5 | INb2 | GT-08151 | INb8 | INb4 |
| GT-08149 | INb8 | INb3 | GT-08343 | INb8 | INb7 |

Note:
in table IN2, the symbols INb1, INb2, INb3, INb4, INb5, INb6, INb7, INb8, INb9, INb10, and INb11 are defined as follows: −20% ≤ INb1 < −15%, −15% < INb2 < −5%, −5% ≤ INb3 ≤ 0%, 0% < INb4 < 5%, 5% ≤ INb5 < 10%, 10% ≤ INb6 < 20%, 20% ≤ INb7 < 30%,30% ≤ INb8 < 50%, 50% ≤ INb9 < 70%, 70% ≤ INb10 < 80%, 80% ≤ INb11 ≤ 100%.

TABLE IN3
Inhibitory activity of the compounds of the present disclosure, designed based on the RSK
inhibitors, on the proliferation of Human acute T lymphoblastic leukemia cell line (Molt-4)

| | Inhibition | Inhibition | | Inhibition | Inhibition |
|---|---|---|---|---|---|
| | Rate at 500 | Rate at 10 | | Rate at 500 | Rate at 10 |
| Comp. No. | nM (%) | nM (%) | Comp. No. | nM (%) | nM (%) |
| BI-D1870 | INc3 | INc1 | GT-08343 | INc9 | INc6 |
| GT-08151 | INc3 | INc5 | GT-08344 | INc11 | INc2 |

Note:
in table IN3, the symbols INcl, INc2, INc3, INc4, INc5, INc6, INc7, INc8, INc9, INc10, and INc11 are defined as follows: 0% < INcl < 5%, 5% ≤ INc2 < 10%, 10% ≤ INc3 < 15%, 15% ≤ INc4 < 20%, 20% ≤ INc5 < 30%, 30% ≤ INc6 < 40%, 40% ≤ INc7 < 50%, 50% ≤ INc8 < 60%, 60% ≤ INc9 < 70%, 70% ≤ INc10 < 80%, 80% ≤ INc11 ≤ 100%.

TABLE IN4
Inhibitory activity of the compounds of the present disclosure, designed based on the RSK
inhibitors, on the proliferation of Human diffuse large B-cell lymphoma cell line (WSU-DLCL2)

| | Inhibition | Inhibition | | Inhibition | Inhibition |
|---|---|---|---|---|---|
| | Rate at 500 | Rate at 10 | | Rate at 500 | Rate at 10 |
| Comp. No. | nM (%) | nM (%) | Comp. No. | nM (%) | nM (%) |
| BI-D1870 | INd5 | INd1 | GT-08151 | INd8 | INd3 |
| GT-08149 | INd7 | INd2 | | | |

Note:
in table IN4, the symbols INd1, INd2, INd3, INd4, INd5, INd6, INd7, INd8, INd9, INd10, and INd11 are defined as follows: 0% < INd1 < 5%, 5% ≤ INd2 < 10%, 10% ≤ INd3 < 15%, 15% ≤ INd4 < 20%, 20% ≤ INd5 < 30%, 30% ≤ INd6 < 40%, 40% ≤ INd7 < 50%, 50% ≤ INd8 < 60%, 60% ≤ INd9 < 70%, 70% ≤ INd10 < 80%, 80% ≤ INd11 ≤ 100%.

TABLE IO1
Inhibitory activity (IC₅₀, nM) of the compounds of Formula
(II) of the present disclosure on tumor cell proliferation

| | H292 cell | | MV-4-11 cell | | MDA-MB-231 cell | |
|---|---|---|---|---|---|---|
| | Inhibition | Inhibition | Inhibition | Inhibition | Inhibition | Inhibition |
| | Rate at 500 | Rate at 10 | Rate at 500 | Rate at 10 | Rate at 500 | Rate at 10 |
| Comp. No. | nM (%) | nM (%) | nM (%) | nM (%) | nM (%) | nM (%) |
| Lenalidomide | IOa1 | IOa4 | IOa3 | IOa5 | IOa1 | IOa1 |
| GT-08118 | IOa5 | IOa5 | IOa8 | IOa7 | IOa5 | IOa5 |

Note:
in table IO1, the symbols IOa1, IOa2, IOa3, IOa4, IOa5, IOa6, IOa7, IOa8, IOa9, IOa10, and IOa11 are defined as follows: 0% < IOa1 < 5%, 5% ≤ IOa2 < 10%, 10% ≤ IOa3 < 15%, 15% ≤ IOa4 < 20%, 20% ≤ IOa5 < 30%, 30% ≤ IOa6 < 40%, 40% ≤ IOa7 < 50%, 50% ≤ IOa8 < 60%, 60% ≤ IOa9 < 70%, 70% ≤ IOa10 < 80%, 80% ≤ IOa11 ≤ 100%.

The results demonstrate that the designed and developed compounds of Formula (I) and Formula (II) of the present invention (including the compounds listed in Table 1, Table 2, and Examples 1-468) effectively inhibit the proliferation of tumor cells (as shown in Tables IA1 to IN4). The inhibitory effect is significantly superior to that of the corresponding positive control drugs.

### II. Western-Blot Assay

Conventional Western blot assay was performed to evaluate the effects of the compounds of the present disclosure on the expression of target proteins in cells. Cell lines selected for WB detection based on the target of interest were summarized in Table II-1.

TABLE II-1

| Cell Type | Cell Line | Corresponding Target(s) |
|---|---|---|
| hPBMC Cell | hPBMC Cell | IKZF1/2/3, Weel, CK1α, GSPT1, IRAK4, CDK2, |
| | | RSK1, RSK2, AKT, EZH2 |
| Adherent Cells | MDA-MB-231 Cell | CDK4/6, CDK9 |
| | LnCap Cell | AR |
| | H1975 Cell | EGFR |
| | H2228 Cell | ALK |
| | MCF-7 Cell | ER |
| | A549 Cell | FAK |
| | T47D Cell | PARP |
| | H4 Cell | tau |
| Suspension Cells | Molt-4 Cell | BCL-2, BRM, BRG1 |
| | K562 Cell | BCR-ABL |
| | Daudi Cell | BTK |

(1) Cell Seeding and Protein Harvesting: The selected cells were processed according to the cell seeding procedure and protein harvesting procedure in Table II-2.

TABLE II-2
Cell Seeding and Protein Harvesting Procedures

| Cell Type | Cell Line | Cell Plating Procedures for Various Cell Types |
|---|---|---|
| hPBMC Cell | hPBMC Cell | Human peripheral blood mononuclear cells (hPBMCs) at a density of |
| | | 1 × 10⁶ cells/mL were thawed according to the PBMC Recovery Protocol |
| | | (Milecell Biotechnologies Inc, Shanghai). The cells were then treated |
| | | with the compounds of the present disclosure (including the compounds |
| | | listed in Tables 1 and 2, and the Examples compounds) at concentrations |
| | | of 50 nM and 500 nM. Controls included a negative control (DMSO) and |
| | | a positive control (the commercial: immunomodulatory drug |
| | | lenalidomide), both processed following the identical experimental |
| | | protocol as the compounds of the present disclosure. After 24 hours of |
| | | compounds treatment, cells were harvested and lysed in RIPA protein |
| | | lysate containing protease inhibitors on ice for 30 min, followed by |
| | | centrifugation. The supernatant was aspirated as the extracted total cell |
| | | protein. The protein concentration of the supernatant was determined |
| | | using the conventional BCA (Bicinchoninic Acid) assay. |
| Adherent Cells | MDA-MB-231 | Adherent cells (e.g., MDA-MB-231 cells) were added to 6-well plates at |
| | Cell, LnCap | a seeding density of 2.5 × 10⁵/mL, with a total volume of 2 mL of cell |
| | Cell, H1975 | suspension per well, cultured for 12 hours, and then treated with the |
| | Cell, H2228 | compounds of the present disclosure (including the compounds listed in |
| | Cell, MCF-7 | Tables 1 and 2, and the Examples compounds) at concentrations of 500 |
| | Cell, A549 | nM and 50 nM. Controls included a negative control (DMSO) and a |
| | Cell, T47D | positive control (corresponding commercial inhibitors), both processed |
| | Cell, H4 Cell, | following the identical experimental protocol as the compounds of the |
| | | present disclosure. After 24 hours of compounds treatment, the culture |
| | | medium was removed. The cells were washed once with PBS and then |
| | | lysed with RIPA protein lysate containing protease inhibitors. The |
| | | resulting lysed proteins were subsequently scraped with a cell scraper, |
| | | placed on ice for 30 minutes, and centrifuged. The supernatant was |
| | | aspirated as the extracted total cell protein. The protein concentration of |
| | | the supernatant was determined using the conventional BCA assay. |
| Suspension | Molt-4 Cell, | Suspension cells (e.g., Molt-4 cells) were added to 12-well plates at a |
| Cells | K562 Cell, | seeding density of 5 × 10⁵/mL, with a total volume of 1 mL of cell |
| | Daudi Cell | suspension per well, cultured for 12 hours, and then treated with the |
| | | compounds of the present disclosure (including the compounds listed in |
| | | Tables 1 and 2, and the Examples compounds) at concentrations of 500 |
| | | nM and 50 nM. Controls included a negative control (DMSO) and a |
| | | positive control (corresponding commercial inhibitors), both processed |
| | | following the identical experimental protocol as the compounds of the |
| | | present disclosure. After 24 hours of compounds treatment, cells were |
| | | harvested and lysed in RIPA protein lysate containing protease inhibitors |
| | | on ice for 30 min, followed by centrifugation. The supernatant was |
| | | aspirated as the extracted total cell protein. The protein concentration of |
| | | the supernatant was determined using the conventional BCA assay. |

(2) To the collected supernatant, 5×SDS sample loading buffer was added, followed by denaturation at 95 °C for 5 minutes. The denatured samples were then separated by SDS-PAGE electrophoresis on a polyacrylamide gel. Proteins were transferred to a nitrocellulose membrane (0.45 μm NC membrane), blocked with blocking buffer at room temperature for 1 hour, and subjected to antibody incubation and development procedures according to the antibody manufacturer's instructions (Cell Signaling Technology).

Half-Maximal Degradation Concentration value (the drug concentration required for degrading proteins by 50%, abbreviated as DC₅₀) reads method: comparing the grayscale values of Western blot bands from drug-treated samples with those from blank DMSO-treated controls, and identifying the drug concentration range at which the grayscale value is half that of the DMSO-treated control bands.

DC₅₀ values can be calculated using ImageJ software to measure grayscale values of Western blot bands from drug-treated samples. The concentration-response curve is fitted to estimate the drug concentration corresponding to half the grayscale value.

Target protein remaining (%) refers to the percentage of target protein remaining in cells after treatment with protein degrader compounds.

Calculation method for target protein remaining (%): Use ImageJ software to measure grayscale values of Western blot bands from drug (protein degrader)-treated samples. The grayscale value of drug-treated bands is divided by that of internal control protein bands, and the resulting quotient is normalized to obtain the relative percentage of remaining target protein after protein degrader treatment.

Target protein degradation rate (%)=1−target protein remaining (%).

The effects of the compounds of the present disclosure (including the compounds listed in Tables 1 and 2, and the Example 1-468 compounds) on substrate proteins expression in cells were shown in Tables IIA1-IIA16 below.

TABLE IIA1
Degradation of substrate protein IRAK4 by the Example compounds
of the present disclosure designed based on IRAK4 inhibitors

| Comp. | | Comp. | | Comp. | | Comp. | | Comp. | |
|---|---|---|---|---|---|---|---|---|---|
| No./names | IRAK4 | No./names | IRAK4 | No./names | IRAK4 | No./names | IRAK4 | No./names | IRAK4 |
| PF06650833 | No | GT- | A | GT- | A | GT- | A | GT- | A |
| | degradation | 05133 | | 05319 | | 05819 | | 08658 | |
| emavusertib | No | GT- | A | GT- | A | GT- | A | GT- | A |
| | degradation | 05142 | | 05325 | | 05822 | | 08659 | |
| GT-04876 | A | GT- | A | GT- | A | GT- | A | GT- | A |
| | | 05143 | | 05326 | | 05825 | | 08660 | |
| GT-04877 | A | GT- | A | GT- | A | GT- | A | GT- | A |
| | | 05144 | | 05285 | | 05827 | | 08661 | |
| GT-04880 | A | GT- | A | GT- | A | GT- | A | GT- | A |
| | | 05122 | | 05290 | | 05833 | | 08662 | |
| GT-04883 | A | GT- | A | GT- | A | GT- | A | GT- | A |
| | | 05123 | | 05291 | | 08647 | | 08664 | |
| GT-04884 | A | GT- | A | GT- | A | GT- | A | GT- | A |
| | | 05188 | | 05748 | | 08648 | | 08666 | |
| GT-04885 | A | GT- | A | GT- | A | GT- | A | GT- | A |
| | | 05220 | | 05749 | | 08650 | | 08667 | |
| GT-04951 | A | GT- | A | GT- | A | GT- | A | GT- | A |
| | | 05221 | | 05806 | | 08651 | | 08668 | |
| GT-04953 | A | GT- | A | GT- | A | GT- | A | GT- | A |
| | | 05238 | | 05813 | | 08652 | | 08669 | |
| GT-05019 | A | GT- | A | GT- | A | GT- | A | GT- | A |
| | | 05239 | | 05814 | | 08653 | | 08670 | |
| GT-05028 | A | GT- | A | GT- | A | GT- | A | GT- | A |
| | | 05240 | | 05815 | | 08654 | | 08671 | |
| GT-05078 | A | GT- | A | GT- | A | GT- | A | GT- | A |
| | | 05241 | | 05816 | | 08655 | | 08672 | |
| GT-05055 | A | GT- | A | GT- | A | GT- | A | GT- | A |
| | | 05242 | | 05817 | | 08656 | | 08673 | |
| GT-05056 | A | GT- | A | GT- | A | GT- | A | | |
| | | 05318 | | 05818 | | 08657 | | | |

TABLE IIA2
Degradation of substrate protein ALK by the Example compounds
of the present disclosure designed based on ALK inhibitors

| Comp. | |
|---|---|
| No./names | ALK |
| GT-04660 | A |
| GT-04661 | A |
| GT-06822 | A |
| GT-06823 | A |
| GT-07298 | A |
| GT-07320 | A |
| GT-07321 | A |
| GT-07322 | A |
| GT-07340 | A |
| GT-07341 | A |
| GT-07342 | A |
| GT-07343 | A |
| GT-07344 | A |
| GT-07362 | A |
| GT-07363 | A |
| GT-08530 | A |

TABLE IIA3
Degradation of substrate protein ER by the Example compounds
of the present disclosure designed based on ER inhibitors

| Comp. No./names | ER |
|---|---|
| GT-04778 | A |

TABLE IIA4
Degradation of substrate protein BTK by the Example compounds
of the present disclosure designed based on BTK inhibitors

| Comp. No./names | BTK |
|---|---|
| GT-05075 | A |
| GT-05077 | A |
| GT-05096 | A |

TABLE IIA5
Degradation of substrate proteins BRM, BRG1 by the
Example compounds of the present disclosure
designed based on SMARCA2/4 inhibitors

| Comp. No./names | BRM | BRG1 |
|---|---|---|
| GT-04952 | A | A |
| GT-04954 | A | A |
| GT-05061 | A | B |
| GT-05068 | A | B |
| GT-05069 | A | A |
| GT-05070 | A | B |
| GT-05071 | A | B |
| GT-05072 | A | B |
| GT-05094 | A | B |
| GT-05095 | A | B |

TABLE IIA6
Degradation of substrate protein EGFR by the Example compounds
of the present disclosure designed based on EGFR inhibitors

| Comp. | | Comp. | | Comp. | | Comp. | | Comp. | |
|---|---|---|---|---|---|---|---|---|---|
| No./names | EGFR | No./names | EGFR | No./names | EGFR | No./names | EGFR | No./names | EGFR |
| GT-05114 | A | GT-05955 | A | GT-06019 | A | GT-06154 | A | GT-06241 | A |
| GT-05836 | A | GT-05946 | A | GT-06035 | A | GT-06159 | A | GT-06315 | A |
| GT-05842 | A | GT-05996 | A | GT-06073 | A | GT-06161 | A | GT-07113 | A |
| GT-05843 | A | GT-05997 | A | GT-06077 | A | GT-06174 | A | | |
| GT-05914 | A | GT-05998 | A | GT-06119 | A | GT-06175 | A | | |

TABLE IIA7
Degradation of substrate proteins CDK4, CDK6
by the Example compounds of the present disclosure
designed based on CDK4/6 inhibitors

| Comp. | | |
|---|---|---|
| No./names | CDK4 | CDk6 |
| GT-05197 | A | B |
| GT-05198 | B | A |
| GT-05199 | B | A |
| GT-05200 | B | A |
| GT-05203 | B | A |
| GT-05204 | B | A |
| GT-05205 | B | A |
| GT-05206 | B | A |
| GT-05812 | B | A |
| GT-05820 | B | A |
| GT-05831 | B | A |
| GT-06336 | A | B |
| GT-06337 | B | A |
| GT-06338 | A | A |
| GT-06487 | A | A |
| GT-06488 | B | A |
| GT-06489 | A | A |
| GT-06497 | A | A |
| GT-06498 | A | A |
| GT-06573 | B | A |
| GT-06586 | A | A |
| GT-06662 | B | A |
| GT-06663 | B | A |
| GT-06673 | B | A |
| GT-06732 | B | A |
| GT-06733 | A | A |
| GT-06734 | A | A |
| GT-06736 | B | A |
| GT-06737 | B | A |
| GT-06837 | A | A |

TABLE IIA8
Degradation of substrate protein AR by the Example compounds
of the present disclosure designed based on AR inhibitors

| Comp. | | Comp. | | Comp. | | Comp. | | Comp. | |
|---|---|---|---|---|---|---|---|---|---|
| No./names | AR | No./names | AR | No./names | AR | No./names | AR | No./names | AR |
| GT-05225 | A | GT-05301 | A | GT-05824 | A | GT-05830 | A | GT-06410 | A |
| GT-05226 | A | GT-05305 | A | GT-05826 | A | GT-05832 | A | GT-06413 | A |
| GT-05229 | A | GT-05821 | A | GT-05828 | A | GT-05834 | A | GT-06414 | A |
| GT-05346 | A | GT-05823 | A | GT-05829 | A | GT-06409 | A | GT-06418 | A |

TABLE IIA9
Degradation of substrate protein CDK2 by the Example compounds
of the present disclosure designed based on CDK2 inhibitors

| Comp. No./names | CDK2 |
|---|---|
| GT-06829 | A |

TABLE IIA10
Degradation of substrate protein FAK by the Example compounds
of the present disclosure designed based on FAK inhibitors

| Comp. | | Comp. | | Comp. | |
|---|---|---|---|---|---|
| No./names | FAK | No./names | FAK | No./names | FAK |
| GT-07468 | A | GT-07470 | A | GT-07661 | A |
| GT-07469 | A | GT-07471 | A | GT-07665 | A |
| GT-07514 | A | GT-07647 | A | GT-07666 | A |

TABLE IIA11
Degradation of substrate protein BCL-2 by the Example compounds
of the present disclosure designed based on BCL-2 inhibitors

| Comp. No./names | BCL-2 |
|---|---|
| GT-07811 | A |
| GT-07812 | A |
| GT-07814 | A |
| GT-07815 | A |
| GT-07816 | A |
| GT-07817 | A |

TABLE IIA12
Degradation of substrate protein EZH2 by the Example compounds
of the present disclosure designed based on EZH2 inhibitors

| Comp. No./names | EZH2 |
|---|---|
| GT-08418 | A |
| GT-08419 | A |
| GT-08420 | A |
| GT-08421 | A |
| GT-08422 | A |
| GT-08423 | A |
| GT-08508 | A |
| GT-08509 | A |
| GT-08510 | A |
| GT-08511 | A |
| GT-08524 | A |
| GT-08525 | A |

TABLE IIA13
Degradation of substrate protein Tau by the Example compounds
of the present disclosure designed based on Tau inhibitors

| Comp. No./names | Tau |
|---|---|
| GT-08568 | A |
| GT-08589 | A |
| GT-08590 | A |
| GT-08591 | A |
| GT-08824 | A |

TABLE IIA14
Degradation of substrate protein AKT by the Example compounds
of the present disclosure designed based on AKT inhibitors

| Comp. No./names | AKT |
|---|---|
| GT-08572 | A |
| GT-08573 | A |
| GT-08574 | A |
| GT-08575 | A |
| GT-08576 | A |
| GT-08577 | A |
| GT-08578 | A |
| GT-08579 | A |
| GT-08580 | A |
| GT-08581 | A |
| GT-08582 | A |
| GT-08583 | A |
| GT-08584 | A |
| GT-08585 | A |
| GT-08586 | A |

TABLE IIA15
Degradation of substrate protein C-ABL by the Example compounds
of the present disclosure designed based on BCR-ABL inhibitors

| Comp. No./names | BCR-ABL |
|---|---|
| GT-08216 | A |
| GT-08219 | A |
| GT-08220 | A |
| GT-08352 | A |
| GT-08356 | A |
| GT-08357 | A |
| GT-08358 | A |
| GT-08359 | A |
| GT-08360 | A |
| GT-08361 | A |

TABLE IIA16
Degradation of substrate proteins WEE1, IKZF1,
IKZF2, IKZF3, CK1α, and GSPT1 by the compounds
of Formula (II) of the present disclosure

| Comp. | | | | | | |
|---|---|---|---|---|---|---|
| No./names | WEE1 | IKZF1 | IKZF2 | IKZF3 | CK1α | GSPT1 |
| Lenalidomide | B | B | B | B | A | B |
| Pomalidomide | B | A | B | A | B | B |
| GT-04511 | B | B | B | B | B | A |
| GT-04536 | B | B | B | B | B | A |
| GT-05567 | A | A | B | A | A | A |
| GT-05570 | A | A | A | A | A | A |
| GT-05575 | A | B | A | A | A | A |
| GT-05581 | A | B | B | A | A | A |
| GT-05592 | B | B | B | A | B | B |
| GT-05593 | B | B | A | B | B | A |
| GT-05591 | B | B | B | A | B | B |
| GT-05582 | A | B | A | A | A | A |
| GT-05588 | B | B | B | A | B | B |
| GT-05589 | B | B | B | A | B | B |
| GT-05580 | A | B | A | A | A | A |
| GT-05590 | B | B | B | A | B | B |
| GT-05594 | B | B | A | B | B | A |
| GT-04990 | A | B | B | B | B | A |
| GT-05595 | B | B | A | B | B | B |
| GT-06979 | A | B | A | B | B | B |
| GT-05568 | A | A | B | A | A | A |
| GT-05576 | A | B | A | A | A | A |
| GT-07968 | A | A | A | A | A | B |

Note: In Tables IIA1 to IIA16, “A” is defined as 0%≤substrate protein remaining %≤80%, and “B” is defined as 80%<substrate protein remaining %≤100%.

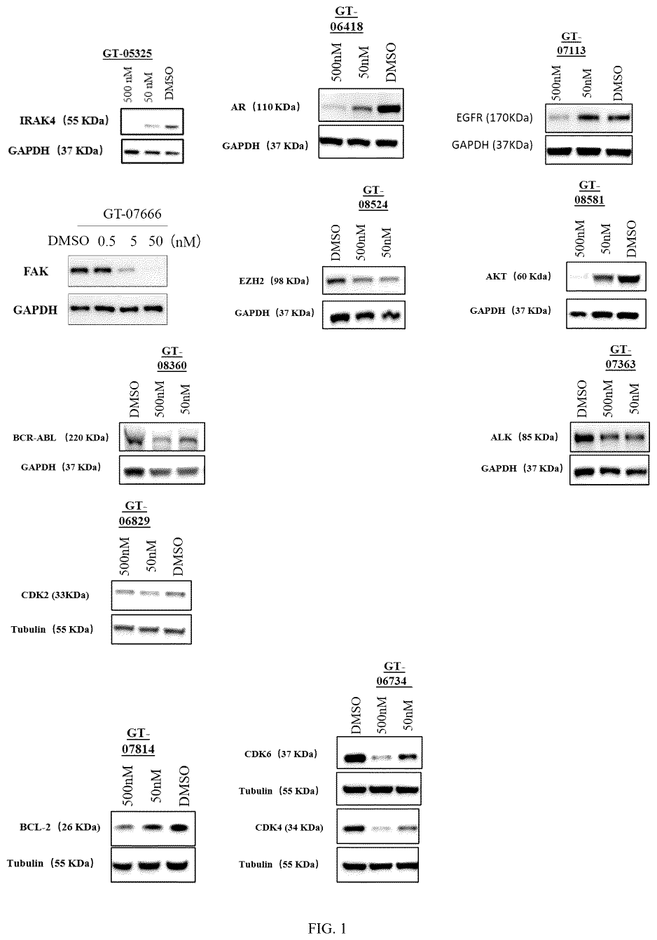

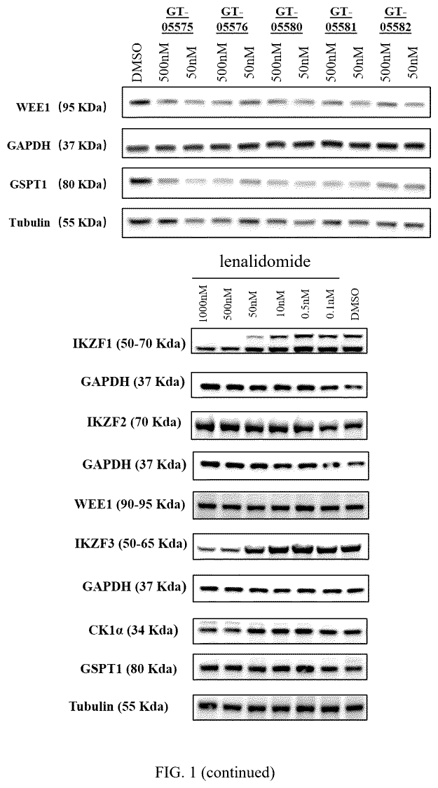

The degradation effects of the designed and synthesized compounds of Formula (I) and Formula (II) of the present invention on the target substrate proteins were shown in Tables IIA1-IIA16 and FIG. 1. From the experimental results in Tables IIA1-IIA16 and FIG. 1, it can be concluded that the compounds of Formula (I) and Formula (II) of the present invention significantly degrade the target substrate proteins. While commercial inhibitors failed to degrade the target proteins, the compounds of Formula (I) of the present invention demonstrated notable degradation efficacy. Compared to lenalidomide, the compounds of Formula (II) of the present invention exhibited significantly enhanced efficiency in inducing substrate protein degradation and also showed selective induction of degradation. In summary, the structural diversity of the compounds of the present invention enables effective degradation of diverse substrate proteins, thereby potentially enabling their use in treating indications associated with these substrates.

### III. Determination of CRBN-Binding Affinity of Compounds

The CEREBLON BINDING kits (specification: 10,000 tests; product number: Catalog #64BDCRBNPEH; CISBIO company) was used to determine the CRBN binding ability of the compounds to be tested through a HTRF method (homogeneous time-resolved fluorescence). The specific methods are as follows:
1. According to the instructions of the CEREBLON BINDING KITS, serial dilutions were performed for the test compounds of the present disclosure (including the compounds in Table 1, Table 2, and Examples 1-468) and for lenalidomide. Specifically, diluent #9 (1×) solution was used as the diluent, and multiple concentration gradients were set. The dilution operation can be performed according to one of the following two methods:
   (1) The first dilution method: The test compounds of the present disclosure (including the compounds in Table 1, Table 2, and Examples 1-468) were subjected to 5-fold serial dilutions starting from a highest concentration of 40 μM, resulting in a total of 7 concentrations from high to low (40, 8, 1.6, 0.32, 0.064, 0.0128, 0.00256 μM). Subsequently, the diluted test compounds and corresponding detection reagents were added to each well of a 96-well plate, resulting in final test compound concentrations of 10, 2, 0.4, 0.08, 0.016, 0.0032, and 0.00064 μM, respectively.
   (2) As an alternative, a second dilution method can be used: The test compounds of the present disclosure (including the compounds in Table 1, Table 2, and Examples 1-468) were subjected to 4-fold gradient dilutions starting from a highest concentration of 8 μM, resulting in 2 concentrations (8 μM, 2 μM). Into the 96-well plate, the diluted test compounds and corresponding detection reagents were added, resulting in final test compound concentrations of 2 μM and 0.5 μM, respectively. The experimental procedure is described below using the diluted 8 μM test compound and lenalidomide solutions as examples.
2. 2.5 μL of the above 8 μM of the tested compounds and lenalidomide solution, as well as the same volume of diluent #9 (1×) solution (solvent control group, Std0) were added to each well of a 96-well plate, respectively. Then, 2.5 μL of human Cereblon WT GST-tagged protein solution was added to each well. Finally, 5 μL of the thoroughly mixed Thalidomide-Re reagent and GST Eu antibody working solution were added to each of the aforementioned wells. The final concentration of the tested compounds and lenalidomide in each well was 2 μM.
3. The blank control wells were sequentially added with 2.5 μL of diluent #9 (1×) solution, 2.5 μL of PROTAC binding buffer, and 5 μL of thoroughly mixed Thalidomide-Re reagent and GST Eu antibody working solution.
4. After sealing and incubating the solutions in the aforementioned wells at room temperature for 3 hours, the absorbance values at emission wavelengths of 620 nm and 665 nm were detected by the HTRF method using a Spark microplate reader (V3.1 SP1).

The corresponding CRBN binding inhibition rate was calculated using the following method:

R_{compound}=OD 665 nm_{compound}/OD 620 nm_{compound}−OD 665 nm_{control}/OD 620 nm_{control}

R_{Std0}=OD 665 nm_{Std0}/OD 620 nm_{Std0}−OD 665 nm_{control}/OD 620 nm_{control} inhibition rate (%)=(1−R_{compound}/R_{Std0})*100

Note: OD 665 nm_{compound} is the absorbance value of each well of the compounds to be tested of the present disclosure at an emission wavelength of 665 nm.
OD 620 nm_{compound} is the absorbance value of each well of the compounds to be tested of the present disclosure at an emission wavelength of 620 nm.
OD 665 nm_{control} is the absorbance value of the blank control well at an emission wavelength of 665 nm.
OD 620 nm_{control} is the absorbance value of the blank control well at an emission wavelength of 620 nm.
OD 665 nm_{Std0} is the absorbance value of the solvent control well at an emission wavelength of 665 nm.
OD 620 nm_{Std0} is the absorbance value of the solvent control well at an emission wavelength of 620 nm.

The test results were shown in Tables III-1 and III-2 below.

TABLE III-1
HTRF inhibitory activity (IC₅₀, μM) of the compounds of the
present disclosure (including but not limited to the compounds
in Tables 1, 2, and the Examples 1-468 compounds)

| | HTRF |
|---|---|
| Comp. No. | IC₅₀ (μM) |
| Lenalidomide | e |
| GT-05568 | c |

Note:
in Table III-1, the symbols a, b, c, d, and e are defined as follows: 0 < a ≤ 0.5 μM, 0.5 μM < b ≤ 1 μM, 1 μM < c ≤ 1.5 μM, 1.5 μM < d ≤ 1.9 μM, 1.9 μM < e ≤ 10 μM, 10 μM < f.

TABLE III-2
Screening of CRBN Binding Affinity of the Compounds of the Present Invention
(including but not limited to the compounds in Tables 1 and 2, and Examples
1-468 Compounds) at Concentrations of 2 μM and 0.5 μM

| | Inhibition | Inhibition | | Inhibition | Inhibition | | Inhibition | Inhibition |
|---|---|---|---|---|---|---|---|---|
| | Rate at | Rate at | | Rate at | Rate at | | Rate at | Rate at |
| | 2 μM | 0.5 μM | Comp. | 2 μM | 0.5 μM | Comp. | 2 μM | 0.5 μM |
| Comp. No. | (%) | (%) | No. | (%) | (%) | No. | (%) | (%) |
| Lenalidomide | a4 | a2 | GT- | a5 | a3 | GT- | a5 | a3 |
| | | | 05201 | | | 07514 | | |
| Brigatinib | a1 | a | GT- | a3 | a3 | GT- | a6 | a3 |
| | | | 05226 | | | 07660 | | |
| alectinib | a | a | GT- | a6 | a4 | GT- | a5 | a2 |
| | | | 05833 | | | 08403 | | |
| GT-04951 | a6 | a3 | GT- | a4 | a3 | GT- | a6 | a1 |
| | | | 05832 | | | 08565 | | |
| GT-05028 | a6 | a2 | GT- | a2 | a3 | GT- | a5 | a1 |
| | | | 05834 | | | 08584 | | |
| GT-05096 | a5 | a1 | GT- | a5 | a1 | GT- | a5 | a1 |
| | | | 05842 | | | 08587 | | |
| GT-05114 | a5 | a2 | GT- | a5 | a1 | GT- | a5 | a2 |
| | | | 06119 | | | 08828 | | |
| GT-05242 | a5 | a3 | GT- | a5 | a1 | GT- | a5 | a2 |
| | | | 06241 | | | 08149 | | |
| GT-05285 | a6 | a2 | GT- | a5 | a1 | GT- | a6 | a2 |
| | | | 06324 | | | 08539 | | |
| GT-05748 | a6 | a1 | GT- | a5 | a3 | GT- | a5 | a1 |
| | | | 06662 | | | 08540 | | |
| GT-05749 | a5 | a2 | GT- | a5 | a2 | GT- | a6 | a3 |
| | | | 06663 | | | 08541 | | |

Note:
in table III-2, the symbols a1, a2, a3, a4, a5, a6, and a7 are defined as follows: 0% < a ≤ 10%, 10% < a1 ≤ 20%, 20% < a2 ≤ 30%, 30% < a3 ≤ 40%, 40% < a4 ≤ 53%, 53% < a5 ≤ 60%, 60% < a6 ≤ 80%, 80% < a7 ≤ 100%.

The results presented in Tables III-1 and III-2 demonstrate that the compounds of the present invention (including but not limited to those listed in Tables 1 and 2, as well as the Examples 1-468 compounds) exhibit strong binding affinity to CRBN.

The results in Table III-1 and Table III-2 demonstrate that the compounds of the present invention (including but not limited to the compounds in Tables 1 and 2, and Examples 1-468) exhibit lower or comparable IC₅₀ values, or higher inhibition rates compared to lenalidomide, indicating stronger or comparable binding affinity to CRBN.

### IV. TNF-α Activity Inhibition Assay

PBMCs cells were cultured at 37 °C with 5% CO₂ and seeded in 96-well plates at 1×10⁷ cells/well. The compounds to be tested (including those listed in Tables 1 and 2, and Examples 1-93 compounds) were dissolved in DMSO and diluted to appropriate concentrations, ensuring that the final DMSO concentration in cell culture did not exceed 0.5%. After 1-hour incubation in medium with or without the compounds, the cells were stimulated with Lipopolysaccharide (LPS, 1 ng/ml) and continued to be cultured for 18-20 hours. Culture supernatants were then collected, diluted with serum-free medium, and analyzed for TNF-α levels using an ELISA kit. IC₅₀ values were calculated using GraphPad Prism 7.0.

TNF-α downregulation is a key mechanism for the anti-tumor action of immunomodulators. The compounds of the present disclosure demonstrated dose-dependent inhibition of TNF-α levels.

### V. Pharmacokinetic Study of the Compounds of the Present Disclosure

The pharmacokinetic properties of the compounds of the present disclosure were evaluated using conventional pharmacokinetic experimental methods.

The test compounds of the present disclosure (including the compounds in Tables 1 and 2, and the Examples 1-93 compounds) were orally administered to experimental animals to evaluate their pharmacokinetic properties. The experimental animals used may be those commonly used in pharmacokinetic experiments, e.g., adult and healthy rodents (e.g., mice, rats (such as Sprague-Dawley (SD) rats), guinea pigs) or non-rodents (rabbits, dogs, and monkeys). The experimental animals used in this study were mice.

The experimental animals were divided into two groups: a control group (for blank plasma collection) and an oral administration group (dosed at 10 mg/kg or 30 mg/kg). Blood samples were collected at predetermined time points after administration, such as 0.25 h, 0.5 h, 1 h, 2 h, 4 h, 8 h, and 24 h post-dose. Blank plasma was collected from the control group.

Prior to analysis, plasma samples were processed as follows: To 10 μL of plasma sample were added 10 μL of 50% acetonitrile and 200 μL of internal standard solution (containing 50 ng/ml dexamethasone in acetonitrile). For blank control plasma samples, no internal standard was added, instead an equivalent volume of acetonitrile was supplemented. The processed samples were vortex-mixed, centrifuged, and the supernatant was subjected to LC-MS/MS analysis.

After preparation of calibration curves and QC samples, the concentrations of compounds in plasma samples were determined by LC-MS/MS analysis. The pharmacokinetic parameters of the test compounds were calculated using the pharmacokinetic calculation software WinNonlin v8.1 with non-compartmental analysis based on the plasma concentration data obtained from LC-MS/MS.

The results demonstrated that the compounds of the present disclosure administered via oral route exhibited favorable drug metabolism and pharmacokinetic (DMPK) properties, indicating their potential as therapeutic agents for cancer patients.

### VI. Effects of the Compounds of the Present Disclosure on TNF-α, IL-10, IL-6, IL-1B, IFN-γ, GM-CSF, IL-2, and IL-12p40 Release from PBMCs Induced by Lipopolysaccharide (LPS) or Phytohemagglutinin (PHA)

The following materials were used in this study:

### VI.1 Reagents and Consumable Materials

| Name | Supplier or Source |
|---|---|
| Human PBMC cell | Shanghai Sai Li |
| Lipopolysaccharide (LPS) | Sigma |
| Phytohemagglutinin (PHA) | MCE |
| RPMI 1640 Medium | Gibco |
| Human TNF-alpha DuoSet ELISA | R&D Systems |
| Human IL-6 DuoSet ELISA | R&D Systems |
| Human IL-10 DuoSet ELISA | R&D Systems |
| Human IFN-gamma DuoSet ELISA | R&D Systems |
| Human IL-2 DuoSet ELISA | R&D Systems |
| Human IL-1 beta ELISA Kit | Abclonal |
| Human GM-CSF ELISA Kit | Abclonal |
| Human IL-12/IL-23 p40 ELISA Kit | Abclonal |

### VI.2 Methods

### VI.2.1 Cell Thawing and Plating

A complete medium was prepared and pre-warmed to 37 °C. Cryovials containing the cells were retrieved from liquid nitrogen, and the area immediately below the cap-notch interface was firmly gripped with forceps. The cryovials were immersed in a 37 °C water bath, and shook occasionally to facilitate thawing. The cell suspension was aspirated and transferred to a centrifuge tube containing 10 mL complete medium, followed by thorough mixing and centrifugation at 400×g for 10 min at room temperature. The supernatant was discarded, and the pellet was resuspended in fresh complete medium. After cell counting and the cell density adjustment, the cell suspension was seeded in 96-well plates at a density of 1×10⁵ cells/well (75 μL/well) and incubated in a incubator for 2 h at 37 °C with 5% CO₂ under high humidity conditions.

### VI.2.2 Compound Preparation and Addition to Cell Plates

1) The test compounds were prepared as 10 mM stock solutions in DMSO.
2) The test compounds were serially diluted 5-fold in DMSO starting from 1 mM (highest concentration) to generate 9 concentration gradients.
3) The test compounds were further diluted in culture medium to five times their final working concentration intended for use.
4) The diluted compounds were added to designated wells according to the plate layout at 25 μL/well, achieving final concentrations starting from 1 μM with 5-fold serial dilution (9 concentration points in total).
5) After 1 h incubation, LPS or PHA (25 μL/well) was added to the cell wells to reach final concentrations of: 1 μg/mL LPS (for TNF-α, IL-10, IL-6, GM-CSF and IL-12p40), 5 μg/mL LPS (IL-1β), 50 μg/mL LPS (IFN-γ), or 1 μg/mL PHA (IL-2).
6) The plates were incubated in a incubator for 24 h at 37 °C with 5% CO₂ under high humidity.

### Cell Plate Layout:

| | 1 | 2 | 3 | 4 | 5 | 6 | 7 | 8 | 9 | 10 | 11 | 12 |
|---|---|---|---|---|---|---|---|---|---|---|---|---|
| A | H₂O | H₂O | H₂O | H₂O | H₂O | H₂O | H₂O | H₂O | H₂O | H₂O | H₂O | H₂O |
| B | H₂O | Cmpd 1# (1 μM Starting, 5-fold dilution, 9 points) | | | | | | | | | LPS/PHA | H₂O |
| C | H₂O | | | | | | | | | | | H₂O |
| D | H₂O | Cmpd 2# (1 μM Starting, 5-fold dilution, 9 points) | | | | | | | | | | H₂O |
| E | H₂O | | | | | | | | | | | H₂O |
| F | H₂O | Cmpd 3# (1 μM Starting, 5-fold dilution, 9 points) | | | | | | | | | Blank | H₂O |
| G | H₂O | | | | | | | | | | | H₂O |
| H | H₂O | H₂O | H₂O | H₂O | H₂O | H₂O | H₂O | H₂O | H₂O | H₂O | H₂O | H₂O |

### VI.2.3 Detection

(1) The cell plate was centrifuged and the culture supernatant was aspirated. TNF-α, IL-10, IL-6, IL-1β, IFN-γ, GM-CSF, IL-2, and IL-12p40 cytokines in the culture supernatant were detected according to the ELISA kit instructions.

The concentrations of TNF-α, IL-10, IL-6, IL-1β, IFN-γ, GM-CSF, IL-2, and IL-12p40 in the samples were calculated based on the standard curve. Inhibition rate %=[(Ac−As)/(Ac−Ab)]×100%
As: OA of the samples (cells+LPS/PHA+test compound)
Ac: OA of positive cell control (cells+LPS/PHA+DMSO)
Ab: OA of blank control (cells+culture medium+DMSO)

(2) IC₅₀ value was calculated as follows: using GraphPad Prism 6 software and applying the calculation formula XY-analysis/Nonlinear regression (curve fit)/Dose response-Inhibition/log (inhibitor) vs. response-Variable slope (four parameters) to perform IC₅₀ curve fitting and determine the IC₅₀ values.

The experimental results demonstrated that the compounds of the present disclosure can modulate the production levels of cytokines associated with autoimmune diseases and may be used for the treatment of autoimmune diseases.

### VII. Efficacy Experiment of the Compounds of the Present Disclosure on Psoriasis-Like Skin Lesions Induced by Imiquimod in Mice

The compounds of the present disclosure (test compounds, including the compounds in Table 1, and Examples) were administered orally via gavage once daily for 7 consecutive days to mice with psoriasis-like skin lesions induced by imiquimod, to investigate their efficacy on such lesions.

A total of 60 female C₅₇BL/6J mice (purchased from Shanghai SLAC Laboratory Animal Co., Ltd.) were randomly divided into 6 groups (n=10 per group) based on body weight:

| | | Num- | | | Adminis- | |
|---|---|---|---|---|---|---|
| | | ber | | Adminis- | tration | Route |
| | | of | | tration | concen- | and |
| | | Ani- | Dose | volume | tration | Dura- |
| Group | Drug | mals | (mg/kg) | (mL/kg) | (mg/mL) | tion |
| 1 | Control: | 10 | NA | 10 | NA | ig. qd. |
| | Vehicle | | | | | 7 days |
| 2 | Model: | 10 | NA | 10 | NA | ig. qd. |
| | Vehicle | | | | | 7 days |
| 3 | Positive | 10 | 3 | 10 | 0.3 | sc. qd. |
| | Control | | | | | 7 days |
| 4 | Low-dose | 10 | 3 | 10 | 0.3 | ig. qd. |
| | test | | | | | 7 days |
| | compounds | | | | | |
| | group | | | | | |
| 5 | Medium- | 10 | 10 | 10 | 1 | ig. qd. |
| | dose test | | | | | 7 days |
| | compounds | | | | | |
| | group | | | | | |
| 6 | High-dose | 10 | 30 | 10 | 3 | ig. qd. |
| | test | | | | | 7 days |
| | compounds | | | | | |
| | group | | | | | |

Note:
ig., oral gavage; sc., subcutaneous injection; qd., once daily.

After grouping the experimental animals, the backs of all mice were shaved. Except for the Control group, the remaining groups were topically treated with imiquimod cream once daily for 7 consecutive days. Simultaneously, each group was administered with a vehicle control, a positive control drug, or the test compounds, according to the above table, once daily for 7 consecutive days.

All clinical symptoms of each animal were observed at the beginning and throughout the experiment. The animals' weights were measured and recorded every 2 days.

### Scoring

Gross Observation Scoring of Skin Lesions (PASI Method): during the last 3 days of modeling and drug administration, the changes in skin lesions of mice in each group were observed once daily. PASI Method: the erythema, scaling, and thickening/infiltration of the lesions were scored separately, and the scores were summed to obtain the total score. PASI Scoring Criteria: 0=none; 1=mild; 2=moderate; 3=severe; 4=very severe.

The skin lesions were photographed once daily during the last 3 days of modeling and drug administration.

After the experiment, the mice were euthanized by CO2 inhalation. Three different areas of skin from the lesion site on the back were collected using a 6 mm punch, weighed, and recorded, and the average weight was calculated.

The skin tissue from the lesion site was homogenized and the levels of IL-23p19, IL-12p40, IL-17, and TNF-α were determined.

The local lesion tissue was fixed, stained with HE, and subjected to pathological scoring (based on epidermal thickness, degree of epidermal keratinization, thickness of the basal layer, and degree of dermal inflammatory cell infiltration). Scoring Criteria: 0=none; 1=mild; 2=moderate; 3=severe; 4=very severe.

Experimental data were presented as Mean+SD. Statistical analysis between two groups was performed using SPSS, with p<0.05 considered statistically significant.

The experimental results demonstrated that the compounds of the present disclosure improve psoriasis-like skin lesions on the back of mice induced by imiquimod and may be used for the treatment of psoriasis.

### VIII. Efficacy Experiment of the Compounds of the Present Disclosure on the CIA Model in Rats Induced by Type II Collagen

The compounds of the present disclosure (test compounds, including the compounds in Table 1, and Examples 1-93) were administered orally via gavage once daily for 21 consecutive days to Wistar rats with collagen-induced arthritis (CIA) that was induced using bovine type II collagen (Chondrex, Inc.), in order to investigate their efficacy in the rat arthritis model.

### Preparation of the Modeling Agent:

10 mg of CII (Immunization Grade Bovine Type II Collagen, purchased from Chondrex, Inc.) was dissolved in 5 mL of 0.05M acetic acid solution, allowing it to be fully dissolved, thereby preparing a solution with a concentration of 2 mg/mL, which was then stored at 4 °C in the dark overnight. On the day of the experiment, the 2 mg/mL CII solution was mixed with an equal volume of a 4 mg/mL solution of Complete Freund's Adjuvant (CFA, also purchased from Chondrex, Inc.) on ice, and was thoroughly emulsified into an emulsion.

On day 0, 10 Wistar rats (purchased from Beijing Vital River Laboratory Animal Technology Co., Ltd.) were randomly selected as the blank control group and were not immunized. The remaining 100 Wistar rats were intradermally injected at the base of the tail with an emulsion prepared by mixing equal volumes of CII (2 mg/mL) and CFA (4 mg/mL) for the first immunization. A second booster immunization was administered to them on day 7. Between days 10 and 14, i.e., 3 to 7 days after the second booster immunization, 50 rats with an arthritis index (AI) score ranging from 1 to 2 were selected from the modeled animals and were divided into 5 groups based on their body weight, foot volume, and AI score, with 10 rats per group:

| | | Num- | | | Adminis- | |
|---|---|---|---|---|---|---|
| | | ber | | Adminis- | tration | Route |
| | | of | | tration | concen- | and |
| | | Ani- | Dose | volume | tration | Dura- |
| Group | Drug | mals | (mg/kg) | (mL/kg) | (mg/mL) | tion |
| 1 | Control: | 10 | NA | 10 | NA | ig. qd. |
| | Vehicle | | | | | 21 days |
| 2 | Model: | 10 | NA | 10 | NA | ig. qd. |
| | Vehicle | | | | | 21 days |
| 3 | Positive | 10 | 0.1 | 10 | 0.01 | sc. qd. |
| | Control | | | | | 21 days |
| 4 | Low-dose | 10 | 3 | 10 | 0.3 | ig. qd. |
| | test | | | | | 21 days |
| | compounds | | | | | |
| | group | | | | | |
| 5 | Medium- | 10 | 10 | 10 | 1 | ig. qd. |
| | dose test | | | | | 21 days |
| | compounds | | | | | |
| | group | | | | | |
| 6 | High-dose | 10 | 30 | 10 | 3 | ig. qd. |
| | test | | | | | 21 days |
| | compounds | | | | | |
| | group | | | | | |

Note:
ig. = oral gavage;
sc. = subcutaneous injection

Administration commenced between 3 and 7 days after the second booster immunization, once the AI score of the affected paws reached 1-2. According to the table above, animals in each group received daily oral gavage (or subcutaneous injection) of either the vehicle control, positive control drug, or the test compounds for 21 consecutive days.

Beginning on day 3 after the second booster immunization, the volume of both hind paws (or toes) was measured and recorded twice weekly.

Foot Volume Measurement: Before the measurement, a line was marked on the rat's ankle joint with a marker pen to indicate the position. After clean water was added to the instrument, the instrument's reading was reset to zero in preparation for the measurement. The hind limb of the rat was placed in the water so that the marked line at the ankle joint was at the surface of the liquid. The reading obtained by pressing the foot pedal at this time was the foot volume of the rat. After the measurement was completed, the foot pedal was pressed again to reset the instrument to zero, preparing it for the measurement of the next rat.

### Scoring

Arthritis Index (AI): The foot volume was measured twice a week, and the swelling of the four toes was scored simultaneously. Each foot was scored on a scale from 0 to 4, with a maximum score of 16 for each rat.

Arthritis Index (AI) Scoring Criteria:

| Score | Degree |
|---|---|
| 0 | No swelling, normal appearance |
| 1 | Mild swelling or redness of the ankle, wrist, or finger |
| | joints |
| 2 | Moderate swelling or redness of the ankle, wrist, or |
| | finger joints |
| 3 | Severe swelling or redness of the ankle, wrist, or finger |
| | joints |
| 4 | Very severe swelling or redness of the ankle, wrist, or |
| | finger joints |

The day of starting drug administration is recorded as Day 0. Five days after starting drug administration, i.e., 2 hours after Day 5 administration, blood was collected from the jugular vein of the experimental animals, allowed to stand for more than 30 minutes, centrifuged to separate the serum, which was stored at −80 °C. The levels of TNF-α, IL-1β, and IL-6 in serum were measured by ELISA.

After the experiment, the animals were euthanized by CO2 inhalation. The spleen and thymus of the animals were collected and weighed, and the organ coefficients were calculated.

Pathological Examination: One side of the foot joint tissue was fixed in 10% neutral formalin solution, followed by decalcification and paraffin embedding to prepare pathological sections. The sections were then stained with hematoxylin and eosin (HE) for pathological observation.

### Observation Indices:

1. Infiltration of synovial inflammatory cells (lymphocytes, plasma cells);
2. Synovial tissue hyperplasia;
3. Synovial pannus;
4. Fibrinoid necrosis on the synovial surface.

Scoring and Counting Method: 0, 1, 2, 3, and 4 levels, where “0” indicates no observation; “1” is mild; “2” is moderate; “3” is severe; and “4” is extremely severe.

Experimental data were presented as Mean+SEM. Data between two groups were analyzed using Excel-T test, with p<0.05 considered statistically significant. Scoring data were analyzed using the non-parametric Mann-Whitney U test in SPSS, with p<0.05 considered statistically significant.

The experimental results demonstrated that the compounds of the present disclosure can regulate the serum levels of inflammatory cytokines in rats with collagen-induced arthritis (CIA) model that was induced using bovine type II collagen, and significantly improve the swelling of rat limbs, and thus may be used for the treatment of arthritis.

The above descriptions represent only preferred embodiments of the present invention, but the scope of protection is not limited thereto. Any person skilled in the art, within the technical scope disclosed by the invention, may make equivalent replacements or modifications based on the technical solutions and inventive concepts of the present invention, and such changes shall fall within the protection scope of the invention.

1. A compound of Formula (I)
PBM-LIN-ULM Formula (I)
or salts, enantiomers, stereoisomers, solvates, isotopically enriched analogs, prodrugs, or polymorphs thereof,
wherein PBM represents a target protein-binding moiety that is covalently linked to ULM via LIN;
wherein ULM represents an E3 ubiquitin ligase-binding ligand moiety, and represents the structure of Formula (ULM): 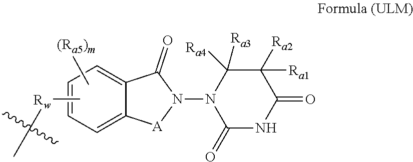
wherein A represents CO, CH₂, or CD₂;
Rₐ₁, Rₐ₂, Rₐ₃, and Rₐ₄ are the same or different and independently represent hydrogen, deuterium, halogen, C₁₋₆ alkyl, halogenated C₁₋₆ alkyl, deuterated C₁₋₆ alkyl, C₁₋₆ alkoxy, deuterated C₁₋₆ alkoxy, or halogenated C₁₋₆ alkoxy;
(Rₐ₅)ₘ indicates that the isoindoline ring to which it is attached is optionally substituted with m Rₐ₅, with each Rₐ₅ being the same or different and each independently representing halogen, hydroxy, mercapto, nitro, amino, cyano, C₁₋₆ alkyl, halogenated C₁₋₆ alkyl, deuterated C₁₋₆ alkyl, C₁₋₆ alkoxy, deuterated C₁₋₆ alkoxy, halogenated C₁₋₆ alkoxy, C₂₋₆ alkenyl, or C₂₋₆ alkynyl;
m represents an integer of 0, 1, 2, 3, or 4;
R_{w} represents O, S, S(O), S(O)₂, alkenylene, alkynylene, or N(Rₐ₆), where Rₐ₆ represents H, or C₁₋₃ alkyl; or R_{w} represents a bond; or
R_{w} represents: 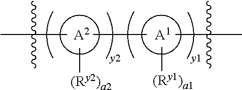
wherein each ring A¹ is the same or different and independently represents nitrogen-containing heterocyclylene, arylene, or heteroarylene, y1 represents an integer of 1 or 2, and (R^{y1})ₐ₁ indicates that each ring A¹ is independently optionally substituted with a1 R^{y1} substituents, wherein each R^{y1} independently represents deuterium, C₁₋₆ alkyl, deuterated C₁₋₆ alkyl, halogenated C₁₋₆ alkyl, C₁₋₆ alkoxy, halogen, amino, hydroxy, mercapto, cyano, carboxyl, C₂₋₆ alkynyl, or C₂₋₆ alkenyl, and a1 represents an integer of 0-20;
each ring A² is the same or different and independently represents heterocyclylene, cycloalkylene, arylene, or heteroarylene, y2 represents an integer of 0 or 1, and (R^{y2})ₐ₂ indicates that each ring A² is independently optionally substituted with a2 R^{y2} substituents, wherein each R^{y2} independently represents deuterium, C₁₋₆ alkyl, deuterated C₁₋₆ alkyl, halogenated C₁₋₆ alkyl, C₁₋₆ alkoxy, halogen, amino, hydroxy, mercapto, cyano, carboxyl, C₂₋₆ alkynyl, or C₂₋₆ alkenyl, and a2 represents an integer of 0-20; and LIN represents: 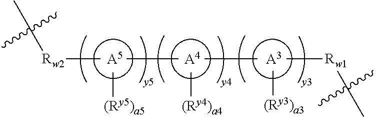
wherein R_{w2} is linked to PBM, and R_{w1} is linked to R_{w};
R_{w2} represents a bond, C(O), C(O)NH, or NHC(O);
each ring A³ is the same or different and independently represents heterocyclylene, cycloalkylene, arylene, or heteroarylene, y3 represents an integer of 0, 1, 2, or 3, and (R^{y3})ₐ₃ indicates that each ring A³ is independently optionally substituted with a3 R^{y3} substituents, with each R^{y3} independently representing deuterium, C₁₋₆ alkyl, deuterated C₁₋₆ alkyl, halogenated C₁₋₆ alkyl, C₁₋₆ alkoxy, halogen, amino, hydroxy, mercapto, cyano, carboxyl, C₂₋₆ alkynyl, or C₂₋₆ alkenyl, and a3 represents an integer of 0-10;
each ring A⁴ is the same or different and independently represents heterocyclylene, cycloalkylene, arylene, or heteroarylene, y4 represents an integer of 0, 1, 2, or 3, and (R^{y4})ₐ₄ indicates that each ring A⁴ is independently optionally substituted with a4 R^{y4} substituents, with each R^{y4} independently representing deuterium, C₁₋₆ alkyl, deuterated C₁₋₆ alkyl, halogenated C₁₋₆ alkyl, C₁₋₆ alkoxy, halogen, amino, hydroxy, mercapto, cyano, carboxyl, C₂₋₆ alkynyl, or C₂₋₆ alkenyl, and a4 represents an integer of 0-10;
each ring A⁵ is the same or different and independently represents heterocyclylene, cycloalkylene, arylene, or heteroarylene, y5 represents an integer of 0, 1, 2, or 3, and (R^{y5})ₐ₅ indicates that each ring A⁵ is independently optionally substituted with a5 R^{y5} substituents, with each R^{y5} independently representing deuterium, C₁₋₆ alkyl, deuterated C₁₋₆ alkyl, halogenated C₁₋₆ alkyl, C₁₋₆ alkoxy, halogen, amino, hydroxy, mercapto, cyano, carboxyl, C₂₋₆ alkynyl, or C₂₋₆ alkenyl, and a5 represents an integer of 0-10;
R_{w1} represents a bond or an optionally substituted linear or branched C₁₋₂₀ alkylene, wherein any one or more methylene groups in the main carbon chain skeleton of the linear or branched C₁₋₂₀ alkylene are optionally replaced by one or more groups selected from Rₐ, R_{b}, and any combination thereof; wherein each Rₐ is independently selected from the group consisting of: O, C(O), OC(O), S, S(O), S(O)₂, S(O)₂N(Rₐ₇), N(Rₐ₇)S(O)₂, C(O)N(Rₐ₇), N(Rₐ₇)C(O), N(Rₐ₇), and N(Rₐ₇)C(O)N(Rₐ₇), where each Raz is independently H or C₁₋₃ alkyl, and when any two or more methylene in the main carbon chain skeleton of the linear or branched C₁₋₂₀ alkylene are replaced by two or more Rₐ groups, the Rₐ groups are not directly connected to each other; and wherein each R^{b} is independently selected from the group consisting of: optionally substituted cycloalkylene, optionally substituted arylene, optionally substituted heterocyclylene, optionally substituted heteroarylene, alkynylene, alkenylene, and any combination thereof.

2. The compound of Formula (I) or salts, enantiomers, stereoisomers, solvates, isotopically enriched analogs, prodrugs, or polymorphs thereof as claimed in claim 1, wherein the PBM represents a small molecule ligand binding to the following protein: epidermal growth factor receptor (EGFR); Cyclin-dependent kinase 4/6(CDK4/6); Cyclin-dependent kinase 2(CDK2); anaplastic lymphoma kinase (ALK); Focal adhesion kinase (FAK); breakpoint cluster region (BCR)-Abelson leukemia virus (ABL)(BCR-ABL) tyrosine kinase; Bruton tyrosine kinase (BTK); Androgen receptor (AR); Estrogen receptor (ER); Bromodomain and extra-terminal domain protein (BET); Interleukin-1 receptor-associated kinase 4 (IRAK4); Cyclin-dependent kinase 9(CDK9); Histone-lysine N-methyltransferase EZH2 (EZH2); B cell lymphoma-2 (BCL-2) family proteins; Cyclin-dependent kinase 2(CDK2); Poly (ADP-ribose) polymerase (PARP); serine/threonine protein kinase (AKT), microtubule-associated protein tau; SWI/SNF related, matrix associated, actin dependent regulator of chromatin, subfamily a, member 2/4 (SMARCA2/4); or ribosomal protein S6 kinase (RSK).

3. The compound of Formula (I) or salts, enantiomers, stereoisomers, solvates, isotopically enriched analogs, prodrugs, or polymorphs thereof as claimed in claim 1, wherein said PBM comprises a structure selected from the following: 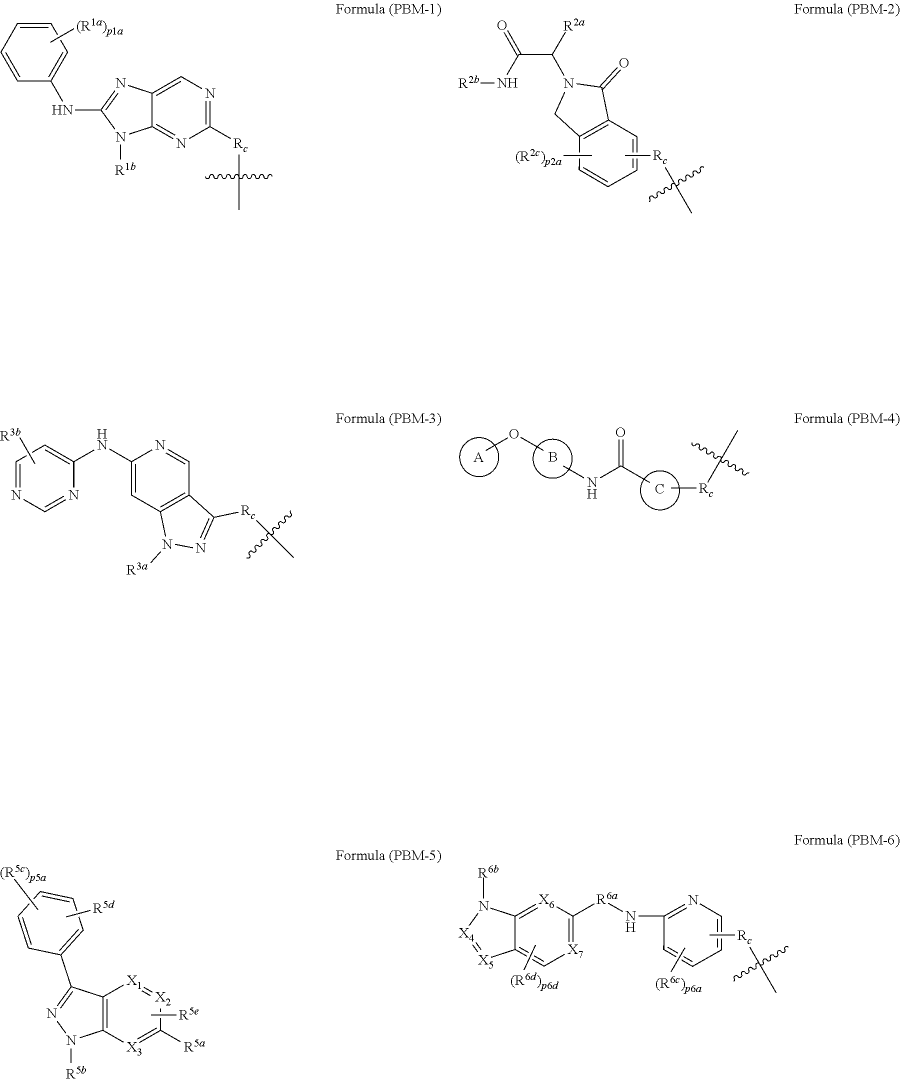 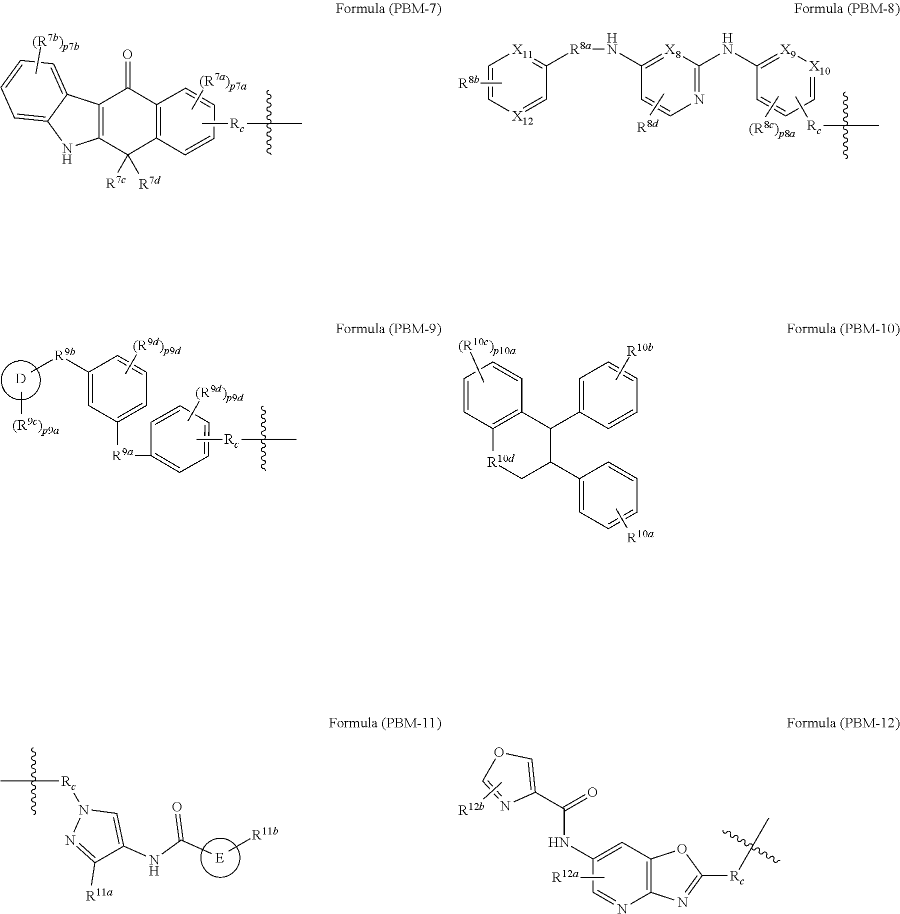 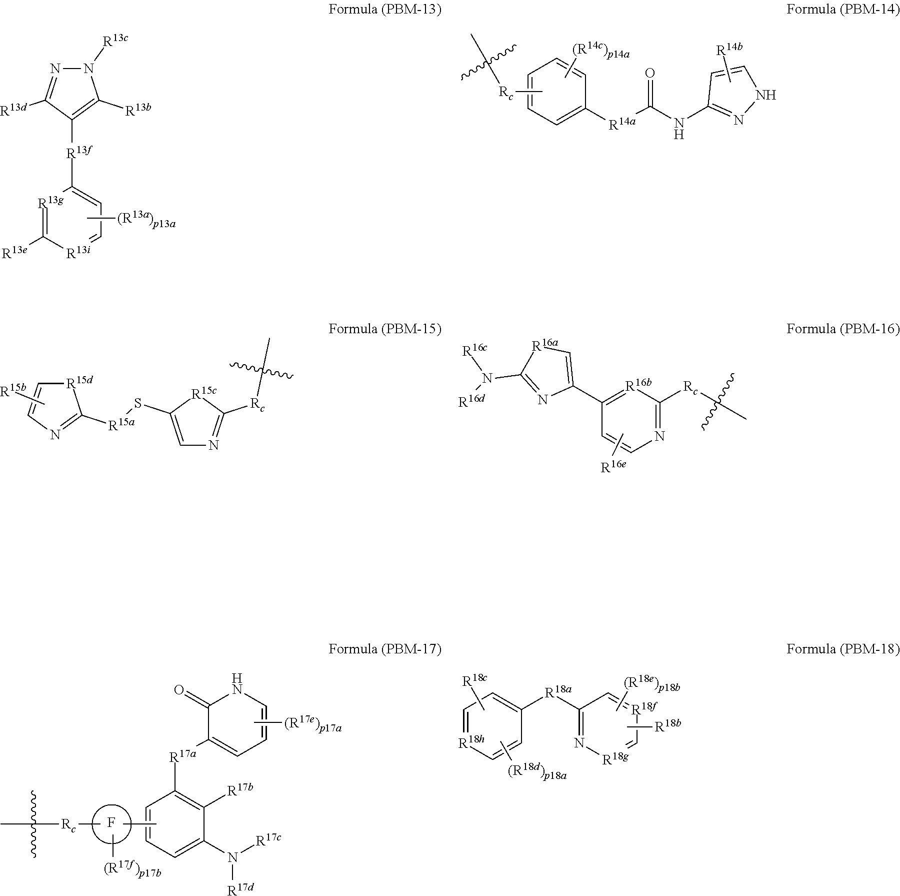 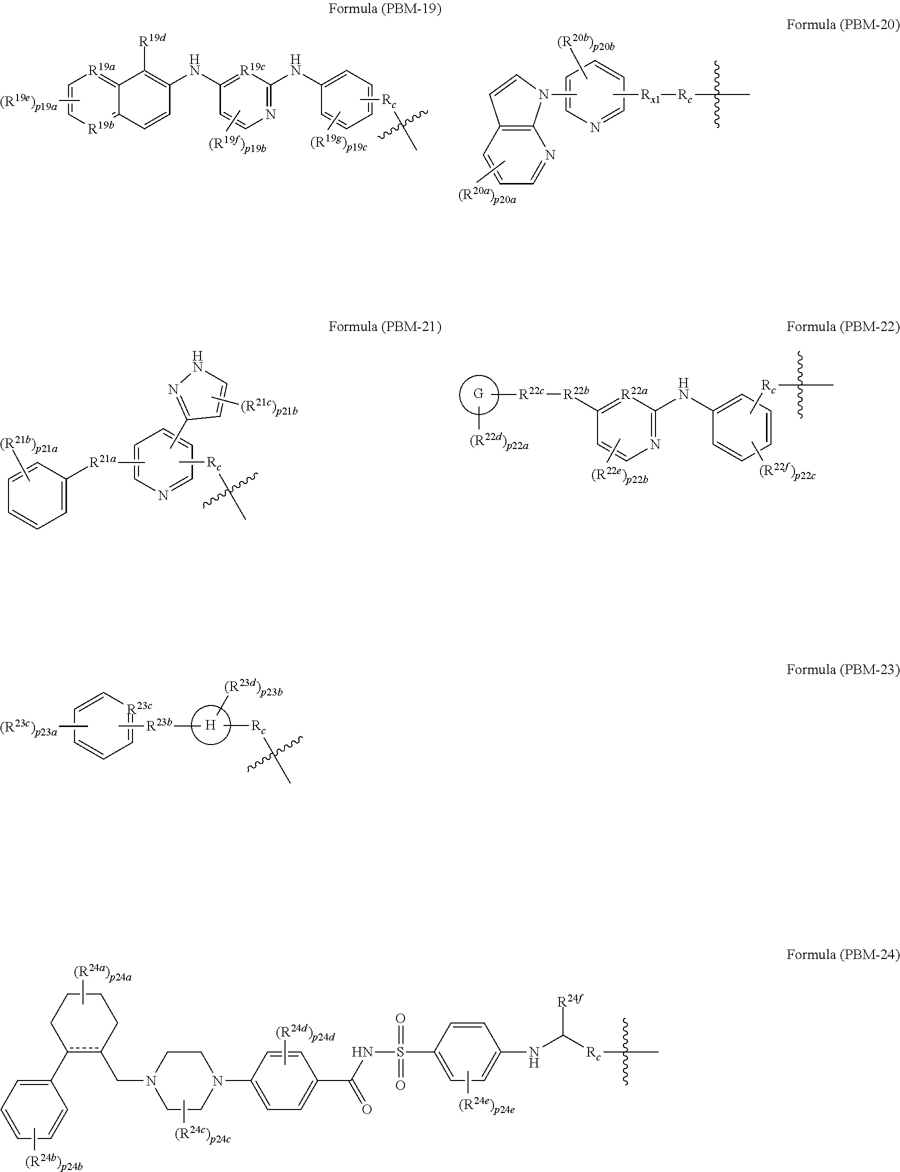 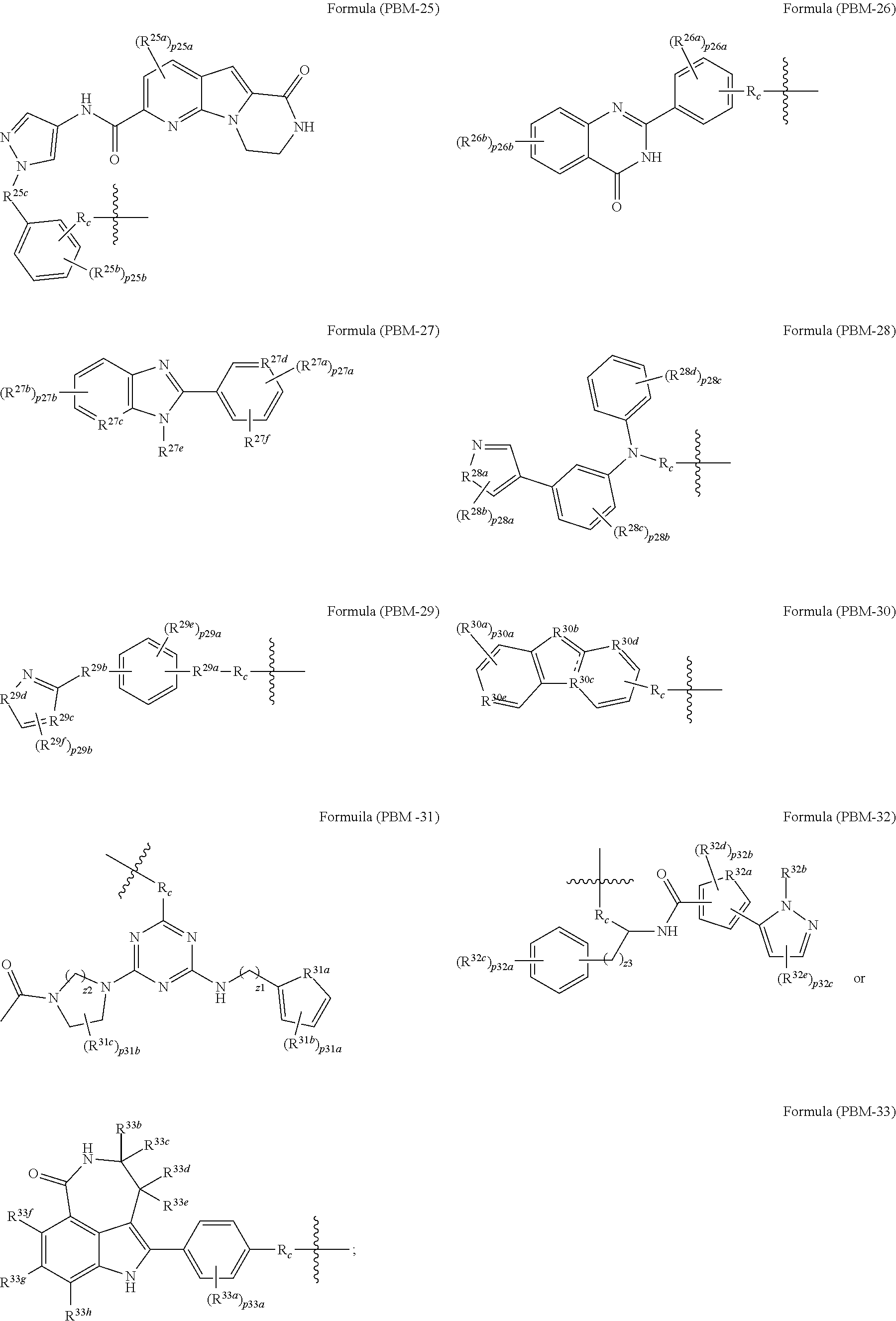
wherein
(R^{1a})ₚ₁ₐ in Formula (PBM-1) indicates that the benzene ring to which it is attached is substituted with p1a R^{1a} groups, with each R^{1a} being the same or different and independently representing deuterium, halogen, hydroxy, C₁₋₆ alkyl, deuterated C₁₋₆ alkyl, halogenated C₁₋₆ alkyl, C₁₋₆ alkoxy, halogenated C₁₋₆ alkoxy, NO₂, NH₂, or cyano;
p1a represents an integer of 0, 1, 2, 3, 4, or 5;
R^{1b} represents substituted or unsubstituted C₃₋₁₅ cycloalkyl, such as cyclopentyl;
R^{2a} in Formula (PBM-2) represents C₆₋₁₀ aryl or 5-to 20-membered heteroaryl, where the C₆₋₁₀ aryl and 5-to 20-membered heteroaryl are each independently optionally substituted with one or more substituents independently selected from the group consisting of deuterium, halogen, hydroxy, cyano, amino, nitro, C₁₋₆ alkyl, halogenated C₁₋₆ alkyl, deuterated C₁₋₆ alkyl, C₁₋₆ alkoxy, halogenated C₁₋₆ alkoxy, and any combination thereof;
R^{2b} represents C₆₋₁₀ aryl or 5-to 20-membered heteroaryl (e.g., 5-to 20-membered heteroaryl containing one or two 5-to 7-membered rings and 1 to 4 heteroatoms selected from N, O, and S), wherein the C₆₋₁₀ aryl and the 5-to 20-membered heteroaryl are each independently optionally substituted with one or more R^{2d}, with each R^{2d} being the same or different and each independently representing deuterium, halogen, hydroxy, cyano, amino, nitro, C₁₋₆ alkyl, halogenated C₁₋₆ alkyl, deuterated C₁₋₆ alkyl, C₁₋₆ alkoxy, or halogenated C₁₋₆ alkoxy;
(R^{2c})ₚ₂ₐ indicates that the isoindoline ring in Formula (PBM-2) is optionally substituted with p2a R^{2c}, with each R^{2c} being the same or different and independently representing deuterium, halogen, hydroxy, C₁₋₆ alkyl, halogenated C₁₋₆ alkyl, deuterated C₁₋₆ alkyl, NO₂, NH₂, C₁₋₆ alkoxy, halogenated C₁₋₆ alkoxy, or cyano;
p2a represents an integer of 0, 1, 2, or 3;
R^{3a} in Formula (PBM-3) represents hydrogen, C₁₋₁₀ alkyl 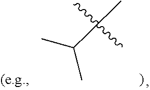 [SMILES: *C(C)C]
deuterated C₁₋₁₀ alkyl, or halogenated C₁₋₁₀ alkyl;
R^{3b} represents 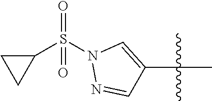 [SMILES: *c1cnn(S(=O)(=O)C2CC2)c1]
or 4-to 12-membered nitrogen-containing heterocyclyl, wherein the 4-to 12-membered nitrogen-containing heterocyclyl is optionally substituted with one or more substituents independently selected from the group consisting of deuterium, halogen, hydroxy, C₁₋₆ alkyl, halogenated C₁₋₆ alkyl, deuterated C₁₋₆ alkyl, C₁₋₆ alkoxy, halogenated C₁₋₆ alkoxy, deuterated C₁₋₆ alkoxy, NO₂, NH₂, cyano, and any combination thereof;
ring A in Formula (PBM-4) represents C₆₋₁₀ aryl or 5-to 20-membered heteroaryl (e.g., 5-to 20-membered heteroaryl containing one or two 5-to 7-membered rings and 1 to 4 heteroatoms selected from N, O, and S), and the ring A is optionally substituted with one or more R^{4a}, with each R^{4a} being the same or different and each independently representing deuterium, halogen, hydroxy, cyano, amino, nitro, C₁₋₆ alkyl, halogenated C₁₋₆ alkyl, deuterated C₁₋₆ alkyl, C₁₋₆ alkoxy, halogenated C₁₋₆ alkoxy, C₂₋₆ alkenyl or C₂₋₆ alkynyl;
ring B in Formula (PBM-4) represents C₃₋₁₅ cycloalkyl or 4-to 20-membered heterocyclyl (e.g., 4-to 20-membered heterocyclyl containing 1 to 4 heteroatoms selected from N, O and S), wherein the ring B is optionally substituted with one or more R^{4b}, with each R^{4b} being the same or different and each independently representing deuterium, halogen, C₁₋₆ alkyl, halogenated C₁₋₆ alkyl, deuterated C₁₋₆ alkyl, C₁₋₆ alkoxy, or halogenated C₁₋₆ alkoxy;
ring C in Formula (PBM-4) represents 5-to 20-membered heteroaryl (e.g., 5-to 20-membered monocyclic or bicyclic heteroaryl containing 1 to 4 heteroatoms selected from N, O and S), wherein the ring C is optionally substituted with one or more R^{4c}, with each R^{4c} being the same or different and each independently representing deuterium, halogen, hydroxy, cyano, amino, nitro, C₁₋₆ alkyl, halogenated C₁₋₆ alkyl, deuterated C₁₋₆ alkyl, C₁₋₆ alkoxy, or halogenated C₁₋₆ alkoxy;
X₁ in Formula (PBM-5) represents N or CR^{5f}, where R^{5f} represents hydrogen, deuterium, or amino, and X₂ represents N or CR^{5g}, where R^{5g} represents hydrogen or R_{c}, and X₃ represents CH or N;
R^{5a} and R^{5b} each independently represent hydrogen or R_{c};
(R^{5c})ₚ₅ₐ in Formula (PBM-5) indicates that the benzene ring to which it is attached is substituted with p5a R^{5c}, with each R^{5c} being the same or different and independently representing —O-aryl, —O-heteroaryl, 4-to 20-membered heterocyclyl, —C₁₋₃ alkylene-NHC(O)-heteroaryl, —C₁₋₃ alkylene-C(O)NH-heteroaryl, C₁₋₆ alkyl, deuterium, deuterated C₁₋₆ alkyl, halogen, or halogenated C₁₋₆ alkyl, wherein the 4-to 20-membered heterocyclyl, aryl, and heteroaryl are each independently optionally substituted with one or more R^{5h}, with each R^{5h} being the same or different and each independently representing deuterium, halogen, hydroxy, cyano, amino, C₁₋₆ alkyl, halogenated C₁₋₆ alkyl, deuterated C₁₋₆ alkyl, C₁₋₆ alkoxy, or halogenated C₁₋₆ alkoxy;
p5a represents an integer of 1, 2, 3, or 4;
R^{5d} represents R_{c}, hydrogen, halogen, C₁₋₆ alkyl, halogenated C₁₋₆ alkyl, deuterated C₁₋₆ alkyl, halogenated C₁₋₆ alkoxy, or C₁₋₆ alkoxy;
R^{5e} represents hydrogen, halogen, cyano, C₁₋₆ alkyl, halogenated C₁₋₆ alkyl, or amino;
wherein R^{5g}, R^{5a}, R^{5b}, and R^{5d} are not simultaneously hydrogen, and only one of R^{5g}, R^{5a}, R^{5b}, and R^{5d} represents R_{c}, wherein
when R^{5a} represents R_{c}, X₂ represents CH or N, and R^{5b} is hydrogen, and R^{5d} represents hydrogen, halogen, C₁₋₆ alkyl, halogenated C₁₋₆ alkyl, deuterated C₁₋₆ alkyl, halogenated C₁₋₆ alkoxy, or C₁₋₆ alkoxy;
when X² represents CR^{5g}, where R^{5g} represents R_{c}, R^{5a} and R^{5b} are hydrogen, and R^{5d} represents hydrogen, halogen, C₁₋₆ alkyl, halogenated C₁₋₆ alkyl, deuterated C₁₋₆ alkyl, halogenated C₁₋₆ alkoxy, or C₁₋₆ alkoxy;
when R^{5b} represents R_{c}, X₂ represents N or CH, and R^{5a} is hydrogen, and R^{5d} represents hydrogen, halogen, C₁₋₆ alkyl, halogenated C₁₋₆ alkyl, deuterated C₁₋₆ alkyl, halogenated C₁₋₆ alkoxy, or C₁₋₆ alkoxy, and
when R^{5d} represents R_{c}, X₂ represents N or CH, and R^{5a} and R^{5b} are hydrogen;
X₄ in Formula (PBM-6) represents CR^{6e} or a fragment 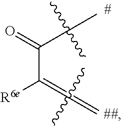
wherein symbol #indicates the point of attachment to N atom adjacent to X₄, symbol ##indicates the point of attachment to X₅, and each R^{6e} independently represents deuterium, hydrogen, halogen, C₁₋₆ alkyl, halogenated C₁₋₆ alkyl, deuterated C₁₋₆ alkyl, —C(O)—C₁₋₆ alkyl, —C(O)-deuterated C₁₋₆ alkyl, or —C(O)NR^{6f}R^{6g}, where R^{6f} and R^{6g} are the same or different and independently represent hydrogen, C₁₋₆ alkyl, or deuterated C₁₋₆ alkyl;
X₅ in Formula (PBM-6) represents N or CR^{6h}, where R^{6h} represents hydrogen, deuterium, halogen, C₁₋₆ alkyl, deuterated C₁₋₆ alkyl, or halogenated C₁₋₆ alkyl;
X₆ and X₇ each independently represent CH or N;
R^{6a} represents a bond or optionally substituted heteroarylene (e.g., heteroarylene substituted with halogen or deuterium);
R^{6b} represents hydrogen, C₁₋₆ alkyl, halogenated C₁₋₆ alkyl, deuterated C₁₋₆ alkyl, or optionally substituted C₃₋₁₂ cycloalkyl;
(R^{6c})ₚ₆ₐ in Formula (PBM-6) indicates that the pyridine ring to which it is attached is optionally substituted with p6a R^{6c}, with each R^{6c} being the same or different and each independently representing deuterium, halogen, C₁₋₆ alkyl, C₁₋₆ alkoxy, halogenated C₁₋₆ alkoxy, deuterated C₁₋₆ alkyl, or halogenated C₁₋₆ alkyl, and p6a represents an integer of 0, 1, 2, or 3;
(R^{6d})_{p6b} in Formula (PBM-6) indicates that the nitrogen-containing bicyclic heteroaryl ring to which it is attached is optionally substituted with p6b R^{6d}, with each R^{6d} being the same or different and each independently representing deuterium, halogen, C₁₋₆ alkyl, C₁₋₆ alkoxy, halogenated C₁₋₆ alkoxy, deuterated C₁₋₆ alkyl, or halogenated C₁₋₆ alkyl, and p6b represents an integer of 0 or 1;
(R^{7a})ₚ₇ₐ in Formula (PBM-7) indicates that the benzene ring to which it is attached is optionally substituted with p7a R^{7a}, with each R^{7a} being the same or different and each independently representing deuterium, cyano, halogen, C₁₋₆ alkyl, C₁₋₆ alkoxy, halogenated C₁₋₆ alkoxy, halogenated C₁₋₆ alkyl, or deuterated C₁₋₆ alkyl, and p7a represents an integer of 0, 1, 2, or 3;
(R^{7b})_{p7b} indicates that the benzene ring to which it is attached is optionally substituted with p7b R^{7b}, with each R^{7b} being the same or different and each independently representing deuterium, cyano, halogen, C₁₋₆ alkyl, C₁₋₆ alkoxy, halogenated C₁₋₆ alkoxy, halogenated C₁₋₆ alkyl, or deuterated C₁₋₆ alkyl, and p7b represents an integer of 0, 1, 2, 3, or 4;
R^{7c} and R^{7d} are the same or different and each independently represent deuterium, C₁₋₆ alkyl, halogen, halogenated C₁₋₆ alkyl, deuterated C₁₋₆ alkyl, C₁₋₆ alkoxy, or halogenated C₁₋₆ alkoxy;
X₈, X₉, X₁₀, X₁₁, and X₁₂ in Formula (PBM-8) are the same or different and independently represent N or CH;
R^{8a} represents a bond or optionally substituted methylene (e.g., halogenated methylene);
R^{8b} represents —C(O)N(R^{8f})R^{8e}, —N(R^{8f})C(O)R^{8e}, —S(O)₂N(R^{8f})R^{8e}, —N(R^{8f})S(O)₂R^{8e}, —S(O)₂R^{8e}, or —P(O)(R^{8e})₂, wherein each R^{8e} is the same or different and each independently represents C₁₋₆ alkyl or deuterated C₁₋₆ alkyl, and R^{8f} represents hydrogen or C₁₋₆ alkyl;
(R^{8c})ₚ₈ₐ in Formula (PBM-8) indicates that the 6-membered ring containing X₉ and X₁₀ to which it is attached is optionally substituted with p8a R^{8c}, with each R^{8c} being the same or different and each independently representing deuterium, halogen, hydroxy, amino, cyano, C₁₋₆ alkoxy, halogenated C₁₋₆ alkoxy, C₁₋₆ alkyl, deuterated C₁₋₆ alkyl, or halogenated C₁₋₆ alkyl;
p8a represents an integer of 0, 1, 2, 3, or 4;
R^{8d} represents deuterium, halogen, hydroxy, amino, cyano, C₁₋₆ alkoxy, halogenated C₁₋₆ alkoxy, C₁₋₆ alkyl, deuterated C₁₋₆ alkyl, or halogenated C₁₋₆ alkyl;
R^{9a} in Formula (PBM-9) represents —NHC(O)— or —C(O)NH—;
R^{9b} represents —NH—, —N(C₁₋₆ alkyl)-, or ethynylene;
ring D in Formula (PBM-9) represents a 5-to 20-membered monocyclic, bicyclic, or polycyclic heteroaryl group containing 1 to 4 heteroatoms selected from N, O, and S, and (R^{9c})ₚ₉ₐ indicates that the ring D is optionally substituted with p9a R^{6c}, with each R^{6c} being the same or different and each independently representing optionally substituted 5-to 15-membered heteroaryl, deuterium, halogen, hydroxy, cyano, amino, C₁₋₆ alkyl, halogenated C₁₋₆ alkyl, deuterated C₁₋₆ alkyl, C₁₋₆ alkoxy, or halogenated C₁₋₆ alkoxy;
p9a represents an integer of 0, 1, 2, or 3;
each (R^{9d})_{p9b} in Formula (PBM-9) independently indicates that the benzene ring to which it is attached is independently optionally substituted with p9b R^{9d}, with each Rod being the same or different and each independently representing deuterium, halogen, hydroxy, cyano, amino, C₁₋₆ alkoxy, halogenated C₁₋₆ alkoxy, C₁₋₆ alkyl, deuterated C₁₋₆ alkyl, or halogenated C₁₋₆ alkyl;
each p9b is the same or different and independently represents an integer of 0, 1, 2, 3, or 4;
R^{10d} in Formula (PBM-10) represents O, S or CH₂;
(R^{10c})ₚ₁₀ₐ indicates that the benzo-fused six-membered ring containing R^{10d} in Formula (PBM-10) is optionally substituted with p10a R^{10c}, with each R^{10c} being the same or different and independently representing deuterium, halogen, hydroxy, cyano, amino, C₁₋₆ alkoxy, halogenated C₁₋₆ alkoxy, C₁₋₆ alkyl, deuterated C₁₋₆ alkyl, or halogenated C₁₋₆ alkyl;
p10a represents an integer of 0, 1, 2, 3, or 4;
only one of R^{10a} and R^{10b} represents R_{c}, and the other represents hydrogen, halogen, hydroxy, cyano, amino, C₁₋₆ alkoxy, halogenated C₁₋₆ alkoxy, C₁₋₆ alkyl, deuterated C₁₋₆ alkyl, or halogenated C₁₋₆ alkyl;
ring E in Formula (PBM-11) represents a 5-to 20-membered monocyclic, bicyclic, or polycyclic heteroarylene group containing 1 to 4 heteroatoms selected from N, O, and S;
R^{11b} represents optionally substituted 5-to 15-membered heteroaryl, optionally substituted 5-to 15-membered heterocyclyl, deuterium, halogen, hydroxy, cyano, amino, C₁₋₆ alkyl, halogenated C₁₋₆ alkyl, deuterated C₁₋₆ alkyl, C₁₋₆ alkoxy, or halogenated C₁₋₆ alkoxy;
R^{11a} represents hydrogen, halogen, C₁₋₆ alkyl, halogenated C₁₋₆ alkyl, deuterated C₁₋₆ alkyl, C₁₋₆ alkoxy, or halogenated C₁₋₆ alkoxy;
R^{12a} in Formula (PBM-12) represents optionally substituted 5-to 15-membered heterocyclyl, optionally substituted 5-to 15-membered heteroaryl, deuterium, halogen, hydroxy, cyano, amino, C₁₋₆ alkyl, halogenated C₁₋₆ alkyl, deuterated C₁₋₆ alkyl, C₁₋₆ alkoxy, or halogenated C₁₋₆ alkoxy;
R^{12b} represents a 5-to 20-membered monocyclic, bicyclic, or polycyclic heteroaryl group containing 1 to 4 heteroatoms selected from N, O, and S, and the heteroaryl is optionally substituted with one or more substituents selected from the group consisting of deuterium, halogen, hydroxy, cyano, amino, C₁₋₆ alkyl, halogenated C₁₋₆ alkyl, deuterated C₁₋₆ alkyl, C₁₋₆ alkoxy, halogenated C₁₋₆ alkoxy, and any combination thereof;
R^{13g} in Formula (PBM-13) represents N or CR^{13h}, where R^{13h} represents hydrogen or halogen; R¹³ⁱ represents N or CR^{13j}, where R^{13j} represents hydrogen or halogen;
R^{13f} represents a bond, —C(O)NH—, —NHC(O)—, —S(O)₂NH—, or —NHS(O)₂—;
(R^{13a})ₚ₁₃ₐ indicates that the 6-membered ring containing R^{13g} and R¹³ⁱ in Formula (PBM-13) is optionally substituted with p13a R^{13a}, with each R^{13a} being the same or different and independently representing deuterium, halogen, hydroxy, cyano, amino, C₁₋₆ alkyl, C₁₋₆ alkoxy, halogenated C₁₋₆ alkoxy, deuterated C₁₋₆ alkyl, or halogenated C₁₋₆ alkyl;
p13a represents an integer of 0, 1 or 2;
R^{13b} represents hydrogen, halogen, C₁₋₆ alkyl, deuterated C₁₋₆ alkyl, halogenated C₁₋₆ alkyl, or —C₁₋₃ alkylene-C₃₋₆ cycloalkyl;
R^{13c} represents hydrogen, C₁₋₆ alkyl, deuterated C₁₋₆ alkyl, or halogenated C₁₋₆ alkyl, or R^{13b} and R^{13c} together with their respective attached nitrogen and carbon atoms form an optionally substituted 5-to 7-membered nitrogen-containing heterocycle;
R^{13d} represents hydrogen or R_{c}, and R^{13e} represents hydrogen or R_{c}, wherein R^{13d} and R^{13e} are not simultaneously hydrogen, and only one of R^{13d} and R^{13e} represents R_{c};
R^{14a} in Formula (PBM-14) represents a bond, C₁₋₃ alkylene, or halogenated C₁₋₃ alkylene;
R^{14b} represents halogen, optionally substituted C₁₋₆ alkyl, C₁₋₆ alkoxy, halogenated C₁₋₆ alkoxy, or optionally substituted C₃₋₁₅ cycloalkyl;
(R^{14c})ₚ₁₄ₐ indicates that the benzene ring in Formula (PBM-14) is optionally substituted with p14a R^{14c}, with each R^{14c} being the same or different and independently representing deuterium, halogen, hydroxy, cyano, amino, C₁₋₆ alkyl, C₁₋₆ alkoxy, halogenated C₁₋₆ alkoxy, deuterated C₁₋₆ alkyl, or halogenated C₁₋₆ alkyl;
p14a represents an integer of 0, 1, 2, 3, or 4;
R^{15a} in Formula (PBM-15) represents C₁₋₃ alkylene, or halogenated C₁₋₃ alkylene;
R^{15b} represents deuterium, C₁₋₆ alkyl, deuterated C₁₋₆ alkyl, halogenated C₁₋₆ alkyl, or optionally substituted C₃₋₆ cycloalkyl;
R^{15c} and R^{15d} each independently represent S or O;
R^{16a} in Formula (PBM-16) represents —S— or a fragment 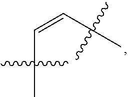 [SMILES: */C=C\*]
wherein when R^{16a} represents —S—, heteroaryl containing R^{16a} and nitrogen atom in Formula (PBM-16) is thiazolyl, and when R^{16a} represents the fragment 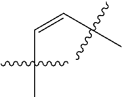 [SMILES: */C=C\*]
the heteroaryl containing R^{16a} and nitrogen atom in Formula (PBM-16) is pyridyl;
R^{16b} represents N or CH;
R^{16c} represents hydrogen or optionally substituted C₁₋₁₀ alkyl;
R^{16d} represents hydrogen, —C₁₋₃ alkylene-optionally substituted 4-to 10-membered heterocyclyl, or optionally substituted 4-to 10-membered heterocyclyl;
R^{16e} represents hydroxy, halogen, C₁₋₆ alkyl, C₁₋₆ alkoxy, halogenated C₁₋₆ alkoxy, deuterated C₁₋₆ alkyl, or halogenated C₁₋₆ alkyl;
R^{17a} in Formula (PBM-17) represents C(O)NH, NHC(O), (CH₂)₁₋₂C(O)NH, (CH₂)₁₋₂NHC(O), C(O)NH(CH₂)₁₋₂, NHC(O)(CH₂)₁₋₂, S(O)₂NH, or NHS(O)₂;
R^{17b} represents hydrogen, hydroxy, halogen, cyano, C₁₋₆ alkoxy, halogenated C₁₋₆ alkoxy, C₁₋₆ alkyl, deuterated C₁₋₆ alkyl, or halogenated C₁₋₆ alkyl;
R^{17c} represents hydrogen, C₁₋₆ alkyl, deuterated C₁₋₆ alkyl, or halogenated C₁₋₆ alkyl, or
R^{17b} and R^{17c}, together with their respective attached carbon and nitrogen atoms and the carbon atom on the benzene ring attached to the nitrogen atom, form an optionally substituted 4-to 6-membered heteroaromatic ring or an optionally substituted 4-to 6-membered heterocycle ring;
R^{17d} represents optionally substituted C₃₋₆ cycloalkyl, C₁₋₆ alkyl, deuterated C₁₋₆ alkyl, halogenated C₁₋₆ alkyl, or optionally substituted 4-to 20-membered heterocyclyl;
(R^{17e})ₚ₁₇ₐ indicates that pyridin-2 (1H)-one ring to which it is attached is substituted with p17a R^{17e}, with each R^{17e} being the same or different and independently representing hydroxy, halogen, cyano, C₁₋₆ alkyl, C₁₋₆ alkoxy, halogenated C₁₋₆ alkoxy, deuterated C₁₋₆ alkyl, or halogenated C₁₋₆ alkyl;
p17a represents an integer of 0, 1, 2, or 3;
ring F in Formula (PBM-17) represents arylene or 5-to 20-membered monocyclic or bicyclic heteroarylene containing from 1 to 4 heteroatoms selected from the group consisting of oxygen, nitrogen, and sulfur; and (R^{17f})_{p17b} indicates that the ring F is optionally substituted with p17b R^{17f}, with each R^{17f} being the same or different and each independently representing deuterium, halogen, hydroxy, cyano, amino, C₁₋₆ alkyl, halogenated C₁₋₆ alkyl, deuterated C₁₋₆ alkyl, C₁₋₆ alkoxy, or halogenated C₁₋₆ alkoxy;
p17b represents an integer of 0, 1, 2, 3, or 4;
R^{18f}, R^{18g}, and R^{18h} in Formula (PBM-18) each independently represent CH or N;
R^{18a} represents a bond, S, or NH;
R^{18b} represents R_{c}, optionally substituted cycloalkyl, or optionally substituted 4-to 15-membered nitrogen-containing heterocyclyl;
R^{18c} represents R_{c}, halogen, amino, hydroxy, cyano, C₁₋₆ alkyl, C₁₋₆ alkoxy, halogenated C₁₋₆ alkoxy, deuterated C₁₋₆ alkyl, halogenated C₁₋₆ alkyl, optionally substituted cycloalkyl, or optionally substituted 4-to 15-membered nitrogen-containing heterocyclyl;
wherein R^{18b} and R^{18c} do not simultaneously represent R_{c}, and only one of R^{18b} and R^{18c} represents R_{c};
(R^{18d})ₚ₁₈ₐ indicates that the 6-membered ring to which it is attached is optionally substituted with p18a R^{18d} with each R^{18d} being the same or different and each independently representing hydroxy, amino, cyano, halogen, C₁₋₆ alkyl, C₁₋₆ alkoxy, halogenated C₁₋₆ alkoxy, deuterated C₁₋₆ alkyl, or halogenated C₁₋₆ alkyl;
p18a represents an integer of 0, 1, 2, 3, or 4;
(R^{18e})_{p18b} indicates that the 6-membered nitrogen-containing heteroaryl ring to which it is attached is optionally substituted with p18b R^{18e}, with each R^{18e} being the same or different and each independently representing hydroxy, amino, cyano, halogen, C₁₋₆ alkyl, C₁₋₆ alkoxy, halogenated C₁₋₆ alkoxy, deuterated C₁₋₆ alkyl, or halogenated C₁₋₆ alkyl;
p^{18b} represents an integer of 0, 1 or 2;
R^{19a}, R^{19b}, and R^{19c} in Formula (PBM-19) each independently represent CH or N;
R^{19d} represents —C(O)NHR^{19h}, —NHC(O)R^{19h}, —S(O)₂NHR^{19h}, —NHS(O)₂R^{19h}, —S(O)₂R^{19h}, or —P(O)(R^{19h})₂, wherein each R^{19h} is the same or different and independently represents C₁₋₆ alkyl, deuterated C₁₋₆ alkyl, or halogenated C₁₋₆ alkyl;
(R^{19e})ₚ₁₉ₐ represents p19a R^{19e} groups, wherein each R^{19e} is the same or different and each independently represents halogen, hydroxy, cyano, amino, nitro, C₁₋₆ alkyl, halogenated C₁₋₆ alkyl, deuterated C₁₋₆ alkyl, C₁₋₆ alkoxy, or halogenated C₁₋₆ alkoxy;
p19a represents an integer of 0, 1, 2, 3, or 4;
(R^{19f})_{p19b} represents p19b R^{19f} groups, wherein each R^{19f} is the same or different and each independently represents halogen, hydroxy, cyano, amino, nitro, C₁₋₆ alkyl, halogenated C₁₋₆ alkyl, deuterated C₁₋₆ alkyl, C₁₋₆ alkoxy, or halogenated C₁₋₆ alkoxy;
p19b represents an integer of 0, 1 or 2;
(R^{19g})_{p19c} indicates that the benzene ring to which it is attached is optionally substituted with p19c R^{19g}, with each R^{19g} each independently representing halogen, hydroxy, cyano, amino, nitro, C₁₋₆ alkyl, halogenated C₁₋₆ alkyl, deuterated C₁₋₆ alkyl, C₁₋₆ alkoxy, or halogenated C₁₋₆ alkoxy;
p^{19c} represents an integer of 0, 1, 2, 3, or 4;
Rₓ₁ in Formula (PBM-20) represents a bond or C(O), or Rₓ₁ represents —C(O)NH—Rₓ₂—C(O)—***, wherein symbol *** indicates the point of attachment to R_{c}, and Rₓ₂ represents optionally substituted C₃₋₁₅ cycloalkyl;
(R^{20a})ₚ₂₀ₐ indicates that the 1H-pyrrolo[2,3-b]pyridine ring to which it is attached is optionally substituted with p20a R^{20a}, wherein each R^{20a} is independently cyano, halogen, hydroxy, amino, nitro, C₁₋₆ alkyl, halogenated C₁₋₆ alkyl, deuterated C₁₋₆ alkyl, C₁₋₆ alkoxy, or halogenated C₁₋₆ alkoxy, and p20a represents an integer of 0, 1, 2, 3, 4, or 5;
(R^{20b})_{p20b} indicates that the pyridine ring to which it is attached is optionally substituted with p20b R^{20b}, wherein each R^{20b} is independently cyano, halogen, hydroxy, nitro, C₁₋₆ alkyl, halogenated C₁₋₆ alkyl, deuterated C₁₋₆ alkyl, C₁₋₆ alkoxy, halogenated C₁₋₆ alkoxy, or NR^{20c}R^{20d}, where R^{20c} and R^{20d} are the same or different and independently represent hydrogen, C₁₋₆ alkyl, deuterated C₁₋₆ alkyl, halogenated C₁₋₆ alkyl, optionally substituted C₃₋₁₅ cycloalkyl, or optionally substituted 4-to 15-membered heterocyclyl, and p20b represents an integer of 0, 1, 2, or 3;
R^{21a} in Formula (PBM-21) represents NHC(O), C(O)NH, NHS(O)₂, or S(O)₂NH;
(R^{21b})ₚ₂₁ₐ indicates that the benzene ring to which it is attached is optionally substituted with p21a R^{21b}, wherein each R^{21b} is independently halogen, hydroxy, cyano, amino, nitro, C₁₋₆ alkyl, halogenated C₁₋₆ alkyl, deuterated C₁₋₆ alkyl, C₁₋₆ alkoxy, or halogenated C₁₋₆ alkoxy, and p21a represents an integer of 0, 1, 2, 3, 4, or 5;
(R^{21c})_{p21b} indicates that the 1H-pyrazole ring to which it is attached is optionally substituted with p21b R^{21c} wherein each R^{21c} is independently halogen, hydroxy, cyano, amino, nitro, C₁₋₆ alkyl, halogenated C₁₋₆ alkyl, deuterated C₁₋₆ alkyl, C₁₋₆ alkoxy, or halogenated C₁₋₆ alkoxy, and p21b represents an integer of 0, 1 or 2;
R^{22a} in Formula (PBM-22) represents N or CH, and R^{22b} represents O, S, NH, CH₂, CH(F), or C(F)₂;
R^{22c} represents a bond or optionally substituted methylene (e.g., halogenated methylene);
ring G represents C₆₋₁₅ aryl, 4-to 15-membered heterocyclyl, or 5-to 15-membered heteroaryl, and (R^{22d})ₚ₂₂ₐ indicates that the ring G to which it is attached is optionally substituted with p22a R^{22d}, wherein each R^{22d} is the same or different and each independently represents deuterium, halogen, hydroxy, cyano, amino, nitro, oxo, C₁₋₆ alkyl, halogenated C₁₋₆ alkyl, deuterated C₁₋₆ alkyl, C₁₋₆ alkoxy, or halogenated C₁₋₆ alkoxy, and p22a represents an integer of 0, 1, 2, 3, or 4;
(R^{22e})_{p22b} indicates that the 6-membered nitrogen-containing heteroaromatic ring containing R^{22a} to which it is attached is optionally substituted with p22b R^{22e}, wherein each R^{22e} independently represents deuterium, halogen, hydroxy, cyano, amino, nitro, C₁₋₆ alkoxy, halogenated C₁₋₆ alkoxy, C₁₋₆ alkyl, deuterated C₁₋₆ alkyl, or halogenated C₁₋₆ alkyl, and p22b represents an integer of 0, 1 or 2;
(R^{22f})_{p22c} indicates that the benzene ring to which it is attached is optionally substituted with p22c R^{22f}, wherein each R^{22f} is the same or different and independently represents deuterium, halogen, hydroxy, cyano, amino, nitro, C₁₋₆ alkoxy, halogenated C₁₋₆ alkoxy, C₁₋₆ alkyl, deuterated C₁₋₆ alkyl, or halogenated C₁₋₆ alkyl, and p22c represents an integer of 0, 1, 2, 3, or 4;
R^{23a} in Formula (PBM-23) represents N or CH;
R^{23b} represents NHC(O), C(O)NH, NHS(O)₂, or S(O)₂NH;
(R^{23c})ₚ₂₃ₐ indicates that the 6-membered ring containing R^{23a} is substituted with p23a R^{23c}, wherein each R^{23c} is the same or different and independently represents halogen, hydroxy, mercapto, cyano, amino, nitro, C₁₋₆ alkyl, halogenated C₁₋₆ alkyl, deuterated C₁₋₆ alkyl, C₁₋₆ alkoxy, or halogenated C₁₋₆ alkoxy, and p23a represents an integer of 0, 1, 2, 3, or 4;
ring H represents 5-to 15-membered heteroarylene, and (R^{23d})_{p23b} indicates that the ring H to which it is attached is optionally substituted with p23b R^{23d}, wherein each R^{23d} is the same or different and independently represents halogen, hydroxy, mercapto, cyano, amino, nitro, C₁₋₆ alkyl, halogenated C₁₋₆ alkyl, deuterated C₁₋₆ alkyl, hydroxy-substituted C₁₋₆ alkyl, amino-substituted C₁₋₆ alkyl, C₁₋₆ alkoxy, halogenated C₁₋₆ alkoxy, or optionally substituted C₃₋₁₅ cycloalkyl, and p23b represents an integer of 0, 1, 2, or 3;
the dashed line --- in Formula (PBM-24) indicates the optional presence of a double bond;
(R^{24a})ₚ₂₄ₐ indicates that the 6-membered cycloalkyl ring to which it is attached is optionally substituted with p24a R^{24a}, wherein each R^{24a} is the same or different and independently represents halogen, hydroxy, mercapto, cyano, amino, nitro, oxo, C₁₋₆ alkyl, halogenated C₁₋₆ alkyl, deuterated C₁₋₆ alkyl, C₁₋₆ alkoxy, or halogenated C₁₋₆ alkoxy, and p24a represents an integer of 0, 1, 2, 3, 4, 5, 6, 7, or 8;
(R^{24b})_{p24b} indicates that the benzene ring to which it is attached is optionally substituted with p24b R^{24b}, wherein each R^{24b} is the same or different and independently represents deuterium, halogen, hydroxy, mercapto, cyano, amino, nitro, C₁₋₆ alkyl, halogenated C₁₋₆ alkyl, deuterated C₁₋₆ alkyl, C₁₋₆ alkoxy, or halogenated C₁₋₆ alkoxy, and p24b represents an integer of 0, 1, 2, 3, 4, or 5;
(R^{24c})_{p24c} indicates that the piperazinylene to which it is attached is optionally substituted with p24c R^{24c} wherein each R^{24c} is the same or different and independently represents halogen, cyano, oxo, C₁₋₆ alkyl, halogenated C₁₋₆ alkyl, deuterated C₁₋₆ alkyl, C₁₋₆ alkoxy, or halogenated C₁₋₆ alkoxy, and p24c represents an integer of 0, 1, 2, 3, 4, 5, 6, 7, or 8;
(R^{24d})ₚ₂₄ₐ indicates that the benzene ring to which it is attached is optionally substituted with p24d R^{24d}, wherein each R^{24d} is the same or different and independently represents halogen, hydroxy, mercapto, C₁₋₆ alkyl, halogenated C₁₋₆ alkyl, deuterated C₁₋₆ alkyl, C₁₋₆ alkoxy, halogenated C₁₋₆ alkoxy, or 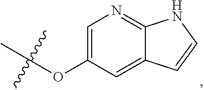 [SMILES: *Oc1cnc2[nH]ccc2c1]
and p24d represents an integer of 0, 1 or 2;
(R^{24e})ₚ₂₄ₑ indicates that the benzene ring to which it is attached is optionally substituted with p24e R^{24e}, wherein each R^{24e} is the same or different and independently represents deuterium, halogen, hydroxy, mercapto, cyano, amino, nitro, —SO₂CF₃, C₁₋₆ alkyl, halogenated C₁₋₆ alkyl, deuterated C₁₋₆ alkyl, C₁₋₆ alkoxy, or halogenated C₁₋₆ alkoxy, and p24e represents an integer of 0, 1, 2, 3, or 4;
R^{24f} represents hydrogen, deuterium, halogen, hydroxy, mercapto, cyano, amino, nitro, C₁₋₆ alkyl, halogenated C₁₋₆ alkyl, deuterated C₁₋₆ alkyl, C₁₋₆ alkoxy, halogenated C₁₋₆ alkoxy, or 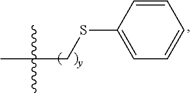
wherein y represents an integer of 1, 2, or 3;
(R^{25a})ₚ₂₅ₐ in Formula (PBM-25) represents p25a R^{25a} groups, wherein each R^{25a} is the same or different and independently represents halogen, hydroxy, mercapto, cyano, amino, nitro, C₁₋₆ alkyl, halogenated C₁₋₆ alkyl, deuterated C₁₋₆ alkyl, C₁₋₆ alkoxy, or halogenated C₁₋₆ alkoxy, and p25a represents an integer of 0, 1, 2, 3, 4, or 5;
(R^{25b})_{p25b} indicates that the benzene ring to which it is attached is optionally substituted with p25b R^{25b}, wherein each R^{25b} is the same or different and independently represents deuterium, halogen, hydroxy, mercapto, cyano, amino, nitro, C₁₋₆ alkyl, halogenated C₁₋₆ alkyl, deuterated C₁₋₆ alkyl, C₁₋₆ alkoxy, or halogenated C₁₋₆ alkoxy, and p25b represents an integer of 0, 1, 2, 3, or 4, and
R^{25c} represents C₁₋₆ alkylene or halogenated C₁₋₆ alkylene;
(R^{26a})ₚ₂₆ₐ in Formula (PBM-26) indicates that the benzene ring to which it is attached is optionally substituted with p26a R^{26a}, wherein each R^{26a} is the same or different and independently represents deuterium, halogen, hydroxy, amino, cyano, C₁₋₆ alkoxy, halogenated C₁₋₆ alkoxy, C₁₋₆ alkyl, deuterated C₁₋₆ alkyl, or halogenated C₁₋₆ alkyl;
p26a represents an integer of 0, 1, 2, 3, or 4;
(R^{26b})_{p26b} indicates that the 4-oxo-3,4-dihydroquinazoline ring to which it is attached is substituted with p26b R^{26b}, wherein each R^{26b} is the same or different and independently represents halogen, hydroxy, amino, cyano, C₁₋₆ alkoxy, halogenated C₁₋₆ alkoxy, C₁₋₆ alkyl, deuterated C₁₋₆ alkyl, or halogenated C₁₋₆ alkyl;
p26b represents an integer of 1, 2, 3, or 4;
(R^{27a})ₚ₂₇ₐ in Formula (PBM-27) indicates that the 6-membered ring containing R^{27d} to which it is attached is optionally substituted with p27a R^{27a}, wherein each R^{27a} is the same or different and independently represents deuterium, halogen, hydroxy, amino, cyano, C₁₋₆ alkoxy, halogenated C₁₋₆ alkoxy, C₁₋₆ alkyl, deuterated C₁₋₆ alkyl, or halogenated C₁₋₆ alkyl;
p27a represents an integer of 0, 1, 2, or 3;
(R^{27b})_{p27b} represents p27b R^{27b} groups, wherein each R^{27b} is the same or different and independently represents deuterium, halogen, hydroxy, amino, cyano, C₁₋₆ alkoxy, halogenated C₁₋₆ alkoxy, C₁₋₆ alkyl, deuterated C₁₋₆ alkyl, halogenated C₁₋₆ alkyl, or optionally substituted C₆₋₁₀ aryl;
p27b represents an integer of 0, 1, 2, or 3;
R^{27c} and R^{27d} each independently represents N or CH;
R^{27e} represents hydrogen or a group having the following structure of Formula (III): 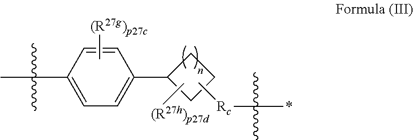
wherein (R^{27g})_{p27c} indicates that the benzene ring to which it is attached is optionally substituted with p27c R^{27g}, with each R^{27g} being the same or different and independently representing deuterium, halogen, hydroxy, amino, cyano, C₁₋₆ alkoxy, halogenated C₁₋₆ alkoxy, C₁₋₆ alkyl, deuterated C₁₋₆ alkyl, or halogenated C₁₋₆ alkyl;
p27c represents an integer of 0, 1, 2, 3, or 4;
n represents an integer of 1, 2, 3, 4, or 5;
(R^{27h})ₚ₂₇ₐ represents p27d R^{27h} groups, wherein each R^{27h} is the same or different and independently represents deuterium, halogen, hydroxy, amino, cyano, C₁₋₆ alkoxy, halogenated C₁₋₆ alkoxy, C₁₋₆ alkyl, deuterated C₁₋₆ alkyl, or halogenated C₁₋₆ alkyl;
p27d represents an integer of 0, 1, 2, 3, 4, 5, 6, or 7, and
symbol * indicates the point of attachment to LIN;
R^{27f} represents hydrogen or R_{c};
wherein when R^{27e} represents hydrogen, R^{27f} represents R_{c}; when R^{27f} represents hydrogen, R^{27e} represents the group having the structure of Formula (III);
R^{28a} represents O, S, or NH;
(R^{28b})ₚ₂₈ₐ indicates that the nitrogen-containing 5-membered ring to which it is attached is optionally substituted with p28a R^{28b}, wherein each R^{28b} is the same or different and independently represents deuterium, halogen, hydroxy, amino, cyano, C₁₋₆ alkoxy, halogenated C₁₋₆ alkoxy, C₁₋₆ alkyl, deuterated C₁₋₆ alkyl, or halogenated C₁₋₆ alkyl;
p28a represents an integer of 0, 1 or 2;
(R^{28c})_{p28b} indicates that the benzene ring to which it is attached is optionally substituted with p28b R^{28c}, wherein each R^{28c} is the same or different and independently represents deuterium, halogen, hydroxy, amino, cyano, C₁₋₆ alkoxy, halogenated C₁₋₆ alkoxy, C₁₋₆ alkyl, deuterated C₁₋₆ alkyl, or halogenated C₁₋₆ alkyl;
p28b represents an integer of 0, 1, 2, 3, or 4;
(R^{28d})_{p28c} indicates that the benzene ring to which it is attached is optionally substituted with p28c R^{28d}, wherein each R^{28d} is the same or different and independently represents deuterium, halogen, hydroxy, amino, cyano, C₁₋₆ alkoxy, halogenated C₁₋₆ alkoxy, C₁₋₆ alkyl, deuterated C₁₋₆ alkyl, halogenated C₁₋₆ alkyl, or optionally substituted C₃₋₁₅ cycloalkyl;
p28c represents an integer of 0, 1, 2, 3, 4, or 5;
R^{29a} represents S(O)₂ or a bond;
R^{29b} represents NH, NHC(O), or C(O)NH;
R^{29c} represents CH or N;
R^{29d} represents N(R^{29h}), wherein R^{29h} represents H, —C(O)-optionally substituted aryl, or —C(O)-optionally substituted heteroaryl, or
R^{29d} represents fragment 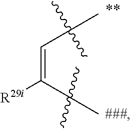
wherein symbol ** indicates the point of attachment to the adjacent nitrogen atom, symbol ###indicates the point of attachment to the adjacent carbon atom, and R²⁹ⁱ represents deuterium, hydrogen, halogen, C₁₋₆ alkyl, halogenated C₁₋₆ alkyl, or deuterated C₁₋₆ alkyl;
(R^{29e})ₚ₂₉ₐ indicates that the benzene ring to which it is attached is optionally substituted with p29a R^{29e}, wherein each R^{29e} is the same or different and independently represents deuterium, halogen, hydroxy, amino, cyano, C₁₋₆ alkoxy, halogenated C₁₋₆ alkoxy, C₁₋₆ alkyl, deuterated C₁₋₆ alkyl, or halogenated C₁₋₆ alkyl;
p29a represents an integer of 0, 1, 2, 3, or 4;
(R^{29f})_{p29b} indicates that the nitrogen-containing ring to which it is attached is substituted by p29b R^{29f} substituents, wherein each R^{29f} is the same or different and independently represents deuterium, amino, halogen, hydroxy, cyano, C₁₋₆ alkoxy, halogenated C₁₋₆ alkoxy, C₁₋₆ alkyl, deuterated C₁₋₆ alkyl, halogenated C₁₋₆ alkyl, —NH-optionally substituted aryl, or —NH-optionally substituted heteroaryl;
p29b represents an integer of 1 or 2;
(R^{30a})ₚ₃₀ₐ in Formula (PBM-30) indicates that that the fused tricyclic ring to which it is attached is optionally substituted by p30a R^{30a} substituents, wherein each R^{30a} is the same or different and independently represents deuterium, halogen, hydroxy, amino, cyano, C₁₋₆ alkoxy, halogenated C₁₋₆ alkoxy, C₁₋₆ alkyl, deuterated C₁₋₆ alkyl, or halogenated C₁₋₆ alkyl; and p30a represents an integer of 0, 1, 2, 3, 4, 5, or 6; [symbol]
in Formula (PBM-30) represents a single bond or a double bond;
fragment 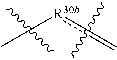
of the fused tricyclic ring in Formula (PBM-30) represents 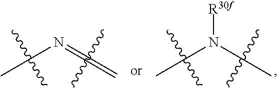
wherein R^{30f} represents hydrogen, C₁₋₆ alkyl, deuterated C₁₋₆ alkyl, or halogenated C₁₋₆ alkyl;
wherein when the fragment 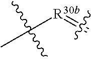
represents 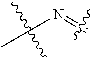
fragment 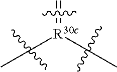
represents 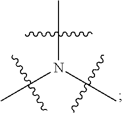 [SMILES: *N(*)*]
when the fragment 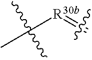
represents 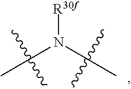
fragment 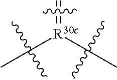
represents 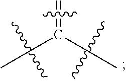
R^{30d} and R^{30e} are the same or different and independently represent N or CH;
R^{31a} in Formula (PBM-31) represents O, S, or NH;
(R^{31b})ₚ₃₁ₐ indicates that the 5-membered ring containing R^{31a} to which it is attached is optionally substituted with p31a R^{31b}, wherein each R^{31b} is the same or different and independently represents deuterium, halogen, hydroxy, amino, cyano, C₁₋₆ alkoxy, halogenated C₁₋₆ alkoxy, C₁₋₆ alkyl, deuterated C₁₋₆ alkyl, or halogenated C₁₋₆ alkyl;
p31a represents an integer of 0, 1, 2, or 3;
(R^{31c})_{p31b} indicates that the diazaheterocyclyl to which it is attached is optionally substituted by p31b R^{31c} substituents, wherein each R^{31c} is the same or different and independently represents deuterium, halogen, hydroxy, amino, cyano, C₁₋₆ alkoxy, halogenated C₁₋₆ alkoxy, C₁₋₆ alkyl, deuterated C₁₋₆ alkyl, or halogenated C₁₋₆ alkyl;
p31b represents an integer of 0, 1, 2, 3, 4, 5, 6, 7, 8, 9, or 10;
Z1 represents an integer of 1, 2, or 3;
Z2 represents an integer of 1, 2, 3, 4, 5, or 6;
R^{32a} in Formula (PBM-32) represents O, S, or NH;
R^{32b} represents hydrogen, C₁₋₆ alkyl, deuterated C₁₋₆ alkyl, or halogenated C₁₋₆ alkyl;
Z3 represents an integer of 1, 2, or 3;
(R^{32c})ₚ₃₂ₐ indicates that the benzene ring to which it is attached is optionally substituted with p32a R^{32c} substituents, wherein each R^{32c} is the same or different and independently represents deuterium, halogen, hydroxy, amino, cyano, C₁₋₆ alkoxy, halogenated C₁₋₆ alkoxy, C₁₋₆ alkyl, deuterated C₁₋₆ alkyl, or halogenated C₁₋₆ alkyl;
p32a represents an integer of 0, 1, 2, 3, 4, or 5;
(R^{32d})_{p32b} indicates that the 5-membered ring containing R^{32a} to which it is attached is optionally substituted with p32b R^{32d} substituents, wherein each R^{32d} is the same or different and independently represents deuterium, halogen, hydroxy, amino, cyano, C₁₋₆ alkoxy, halogenated C₁₋₆ alkoxy, C₁₋₆ alkyl, deuterated C₁₋₆ alkyl, or halogenated C₁₋₆ alkyl;
p32b represents an integer of 0, 1 or 2;
(R^{32e})_{p32c} indicates that the pyrazole ring to which it is attached is optionally substituted by p32c R^{32e} substituents, wherein each R^{32e} is the same or different and independently represents deuterium, halogen, hydroxy, amino, cyano, C₁₋₆ alkoxy, halogenated C₁₋₆ alkoxy, C₁₋₆ alkyl, deuterated C₁₋₆ alkyl, or halogenated C₁₋₆ alkyl;
p32c represents an integer of 0, 1 or 2;
(R^{33a})ₚ₃₃ₐ indicates that the benzene ring to which it is attached is optionally substituted with p33a R^{33a} substituents, wherein each R^{33a} is the same or different and independently represents deuterium, halogen, hydroxy, amino, cyano, C₁₋₆ alkoxy, halogenated C₁₋₆ alkoxy, C₁₋₆ alkyl, deuterated C₁₋₆ alkyl, or halogenated C₁₋₆ alkyl;
p33a represents an integer of 0, 1, 2, 3, 4, or 5;
R^{33b}, R^{33c}, R^{33d}, R^{33e}, R^{33f}, R^{33g}, and R^{33h} in Formula (PBM-33) are the same or different and independently represent hydrogen, deuterium, halogen, hydroxy, amino, cyano, C₁₋₆ alkoxy, halogenated C₁₋₆ alkoxy, C₁₋₆ alkyl, deuterated C₁₋₆ alkyl, or halogenated C₁₋₆ alkyl; and
R_{c} in each general formula independently represents a bond, —O—, —N(Rₐ)—, —NHC(O)—*, —C(O)NH—*, —N(R_{d})—R_{f}—N(Rₑ)—*, —R_{f}—C(O)NH—*, —R_{f}—NHC(O)—*, —R_{f}—C(O)O—*, —R_{f}—OC(O)—*, —C(O)O—*, —OC(O)—*, —R_{f}—, —R_{f}—N(R_{d})—*, —O—R_{f}—N(R_{d})—*, 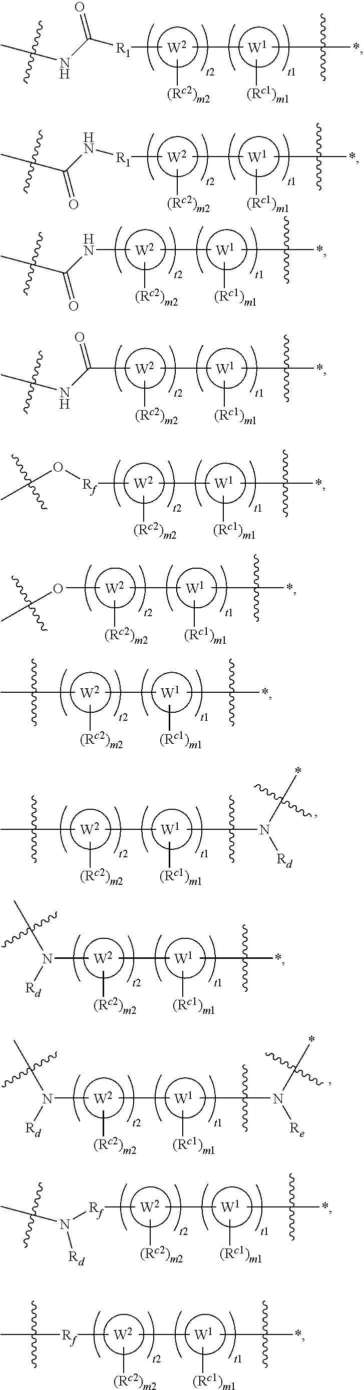 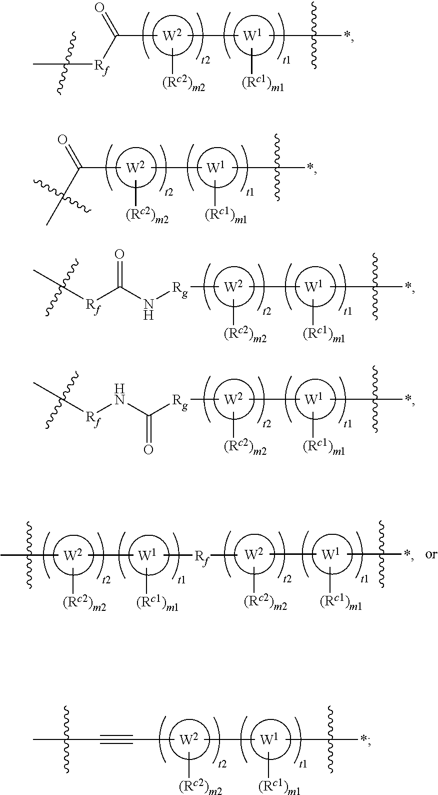
wherein each ring W¹ is the same or different and each independently represents nitrogen-containing heterocyclylene, t1 represents an integer of 1 or 2, and (R_{c1})ₘ₁ indicates that each ring W¹ is independently optionally substituted with m1 R^{c1} groups, wherein each R^{c1} is independently deuterium, C₁₋₆ alkyl, deuterated C₁₋₆ alkyl, halogenated C₁₋₆ alkyl, C₁₋₆ alkoxy, halogen, amino, hydroxy, mercapto, cyano, carboxyl, C₂₋₆ alkynyl, C₂₋₆ alkenyl, optionally deuterated C₃₋₆ cycloalkyl, or R_{c4}—R_{c3}—, wherein R_{c3} is optionally substituted C₁₋₆ alkylene, or optionally substituted C₂₋₆ alkenylene, and R_{c4} represents halogen, R_{c5}R_{c6}N—, hydroxy, carboxyl, ethynyl, or vinyl, wherein Rₑ5 and R_{c6} are the same or different and independently represent hydrogen or C₁₋₃ alkyl, and m1 represents an integer of 0-20;
each ring W² is the same or different and each independently represents heterocyclylene, cycloalkylene, arylene, or heteroarylene, t2 represents an integer of 0 or 1, and (R_{c2})ₘ₂ indicates that each ring W² is independently optionally substituted with m2 R_{c2} groups, wherein each R_{c2} is independently deuterium, C₁₋₆ alkyl, deuterated C₁₋₆ alkyl, halogenated C₁₋₆ alkyl, C₁₋₆ alkoxy, halogen, amino, hydroxy, mercapto, cyano, carboxyl, C₂₋₆ alkynyl, C₂₋₆ alkenyl, optionally deuterated C₃₋₆ cycloalkyl, or R_{c8}—R_{c7}—, wherein R_{c7} is optionally substituted C₁₋₆ alkylene or optionally substituted C₂₋₆alkenylene, and R_{c8} represents halogen, R_{c9}R_{c10}N—, hydroxy, carboxyl, ethynyl, or vinyl, wherein R_{c9} and R_{c10} are the same or different and independently represent hydrogen or C₁₋₃ alkyl, and m2 represents an integer of 0-20; and
R_{d} and Rₑ each independently represent hydrogen or C₁₋₆ alkyl; R_{f} and R_{g} each independently represent optionally substituted C₁₋₆ alkylene or optionally substituted C₂₋₆ alkenylene, and symbol * indicates the point of attachment to LIN.

4. The compound of Formula (I) or salts, enantiomers, stereoisomers, solvates, isotopically enriched analogs, prodrugs, or polymorphs thereof as claimed in claim 3, wherein the PBM represents a structure of the following formulae: 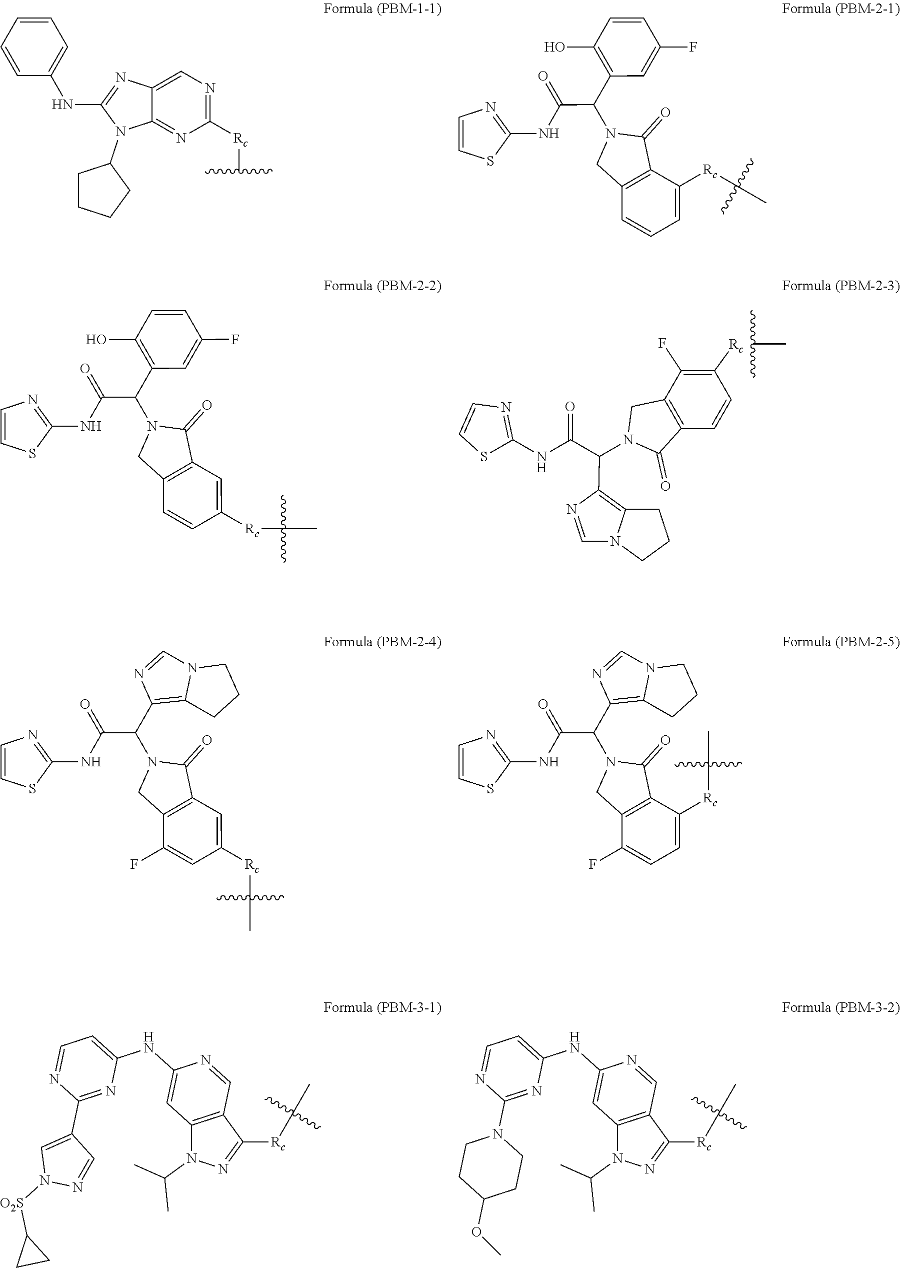 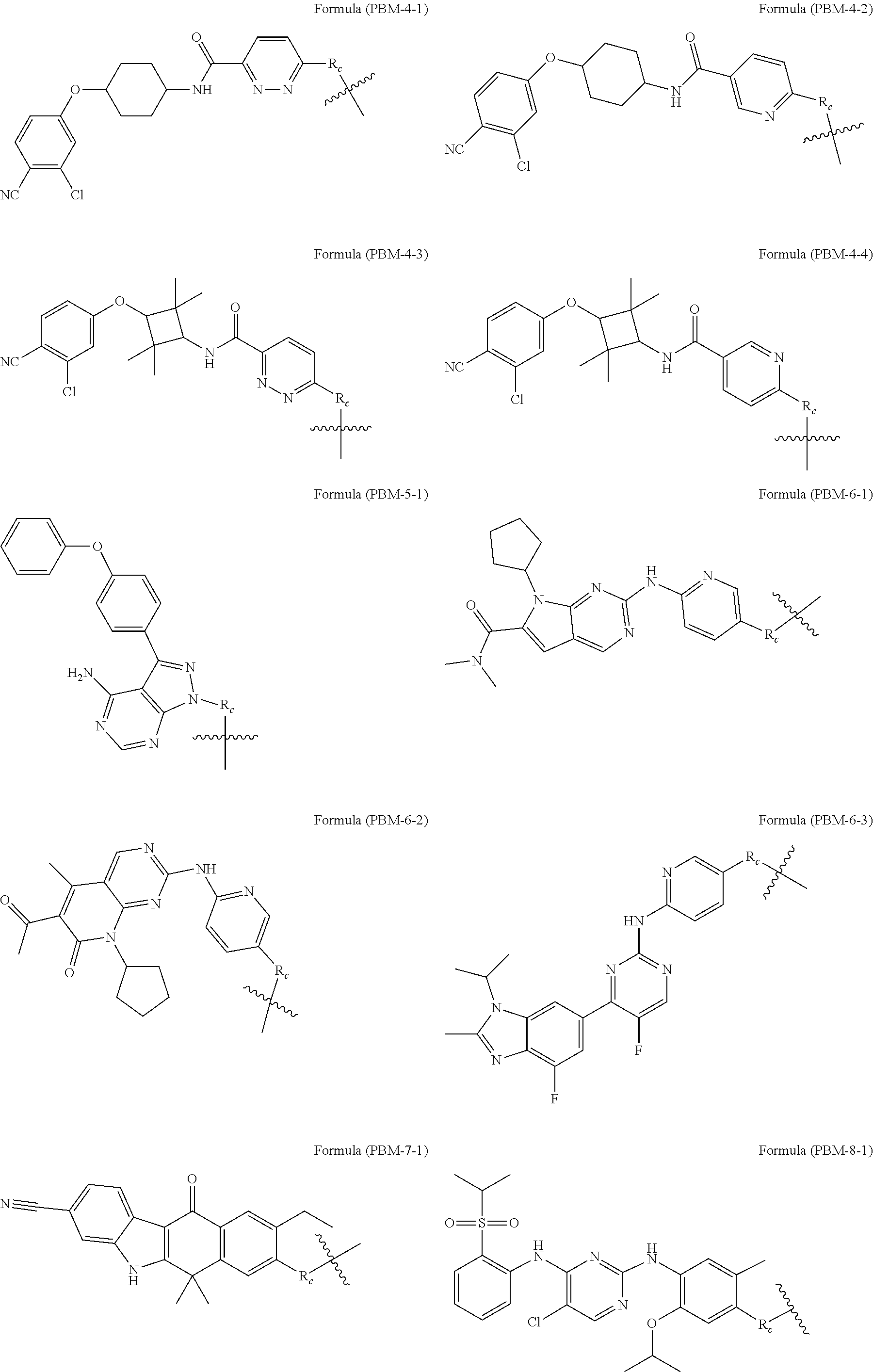 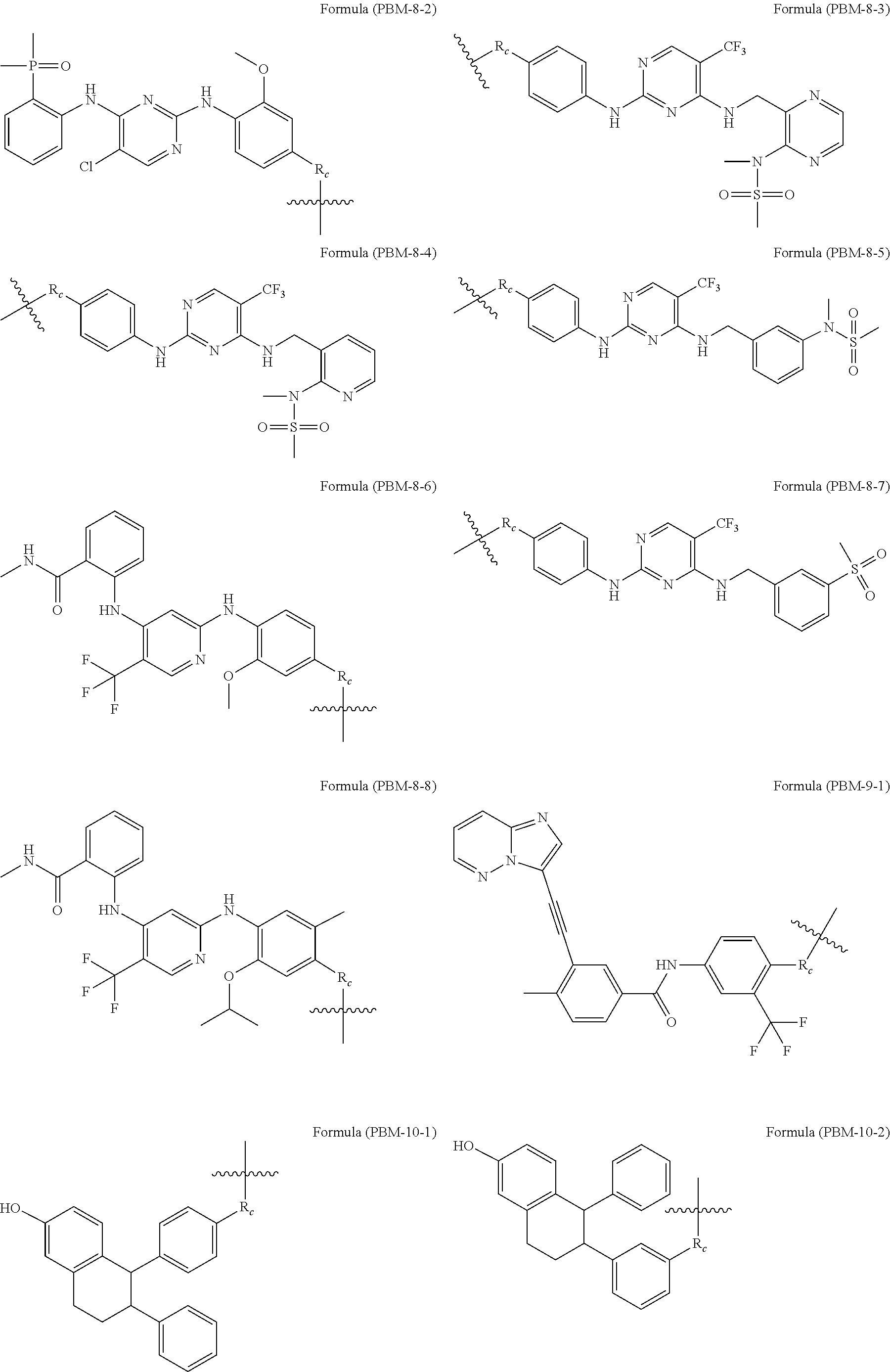 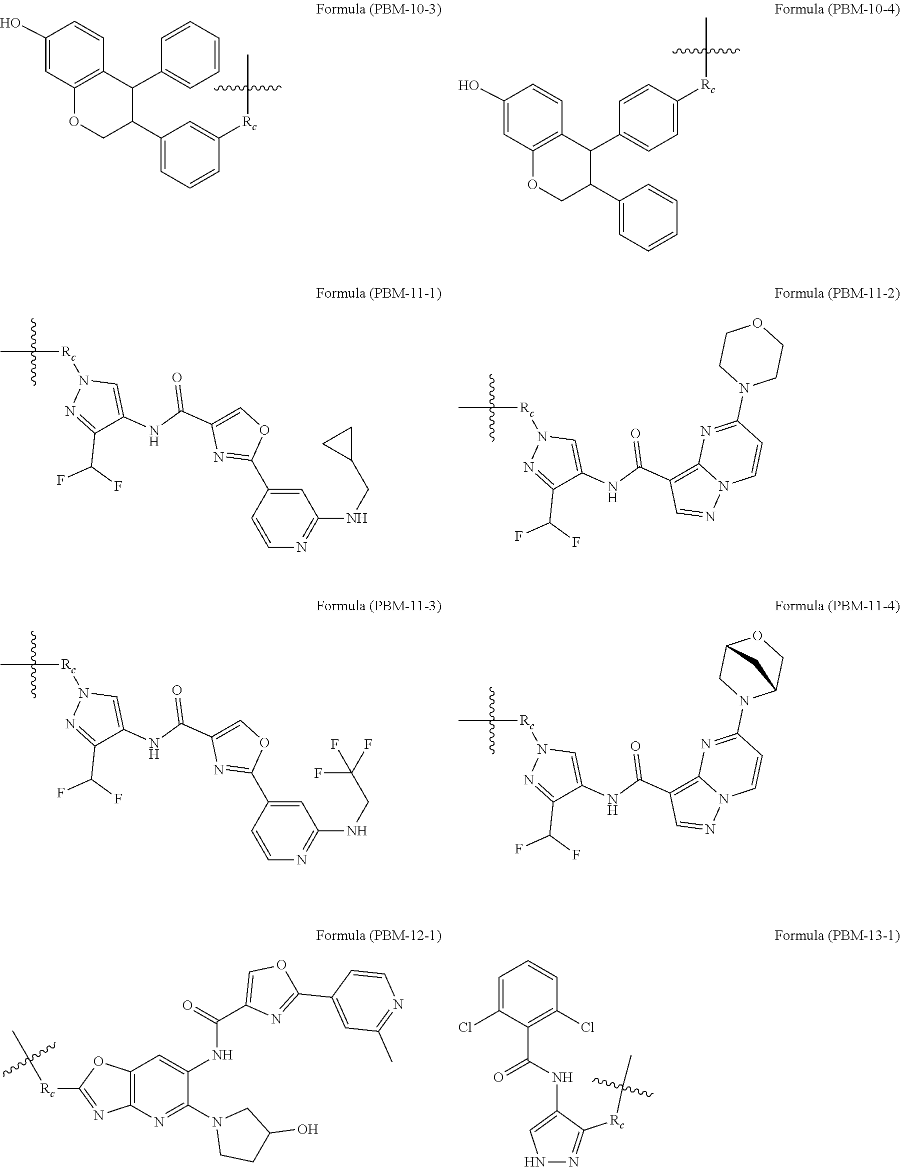 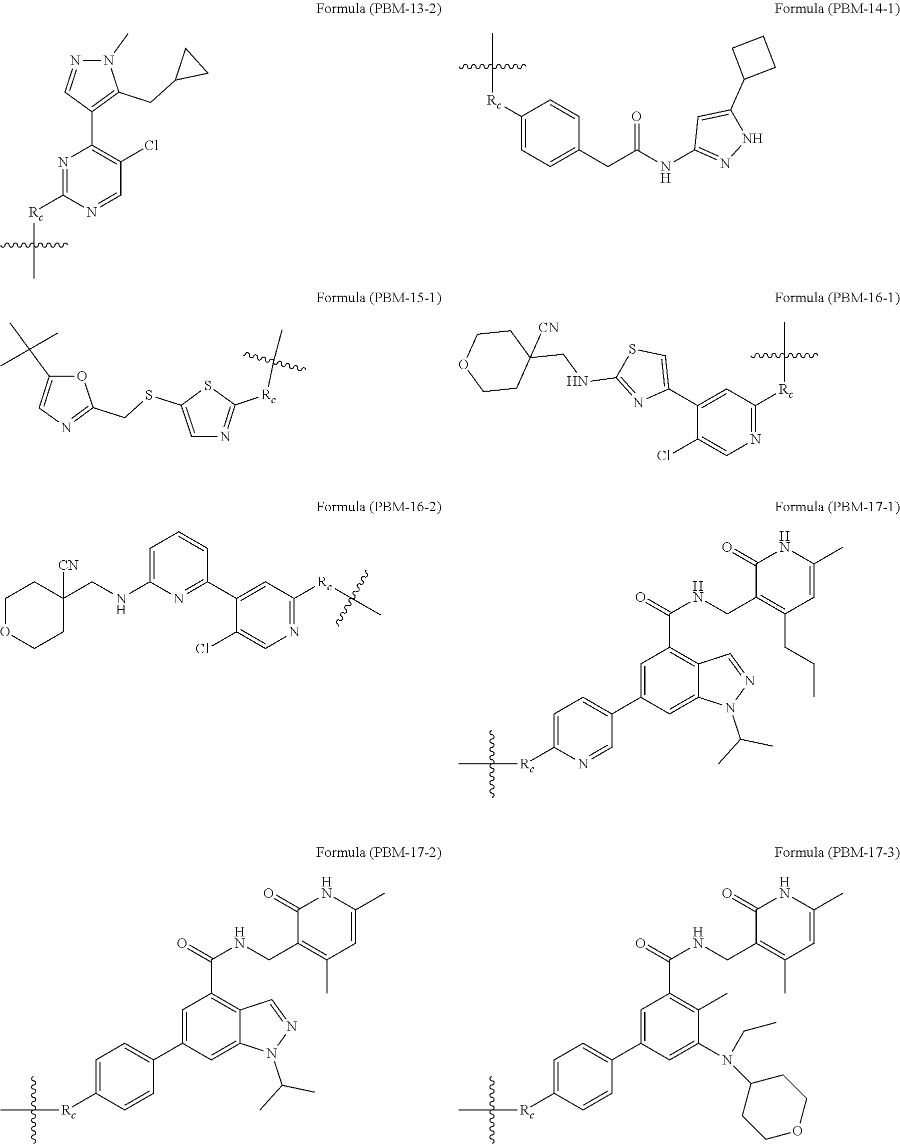 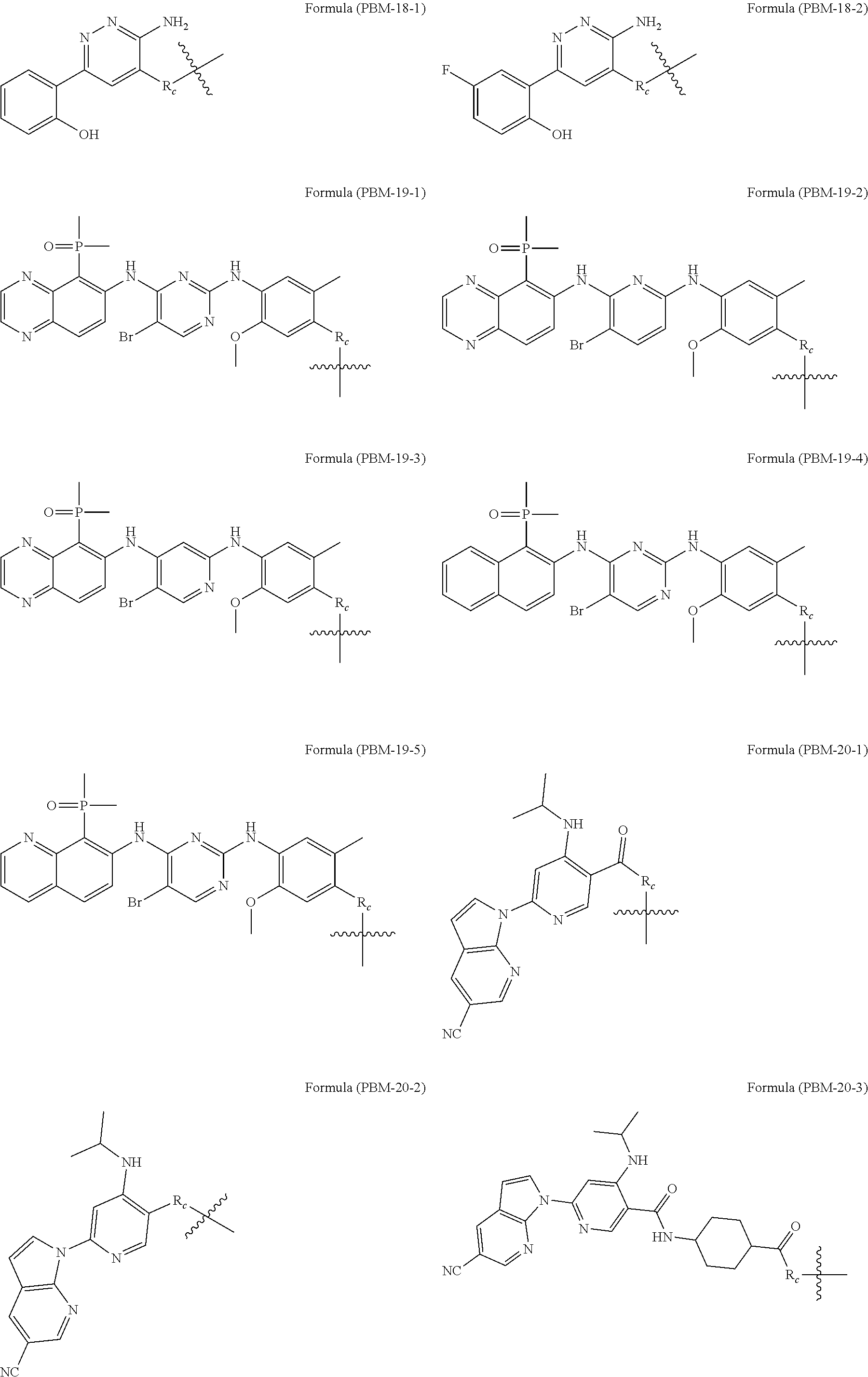 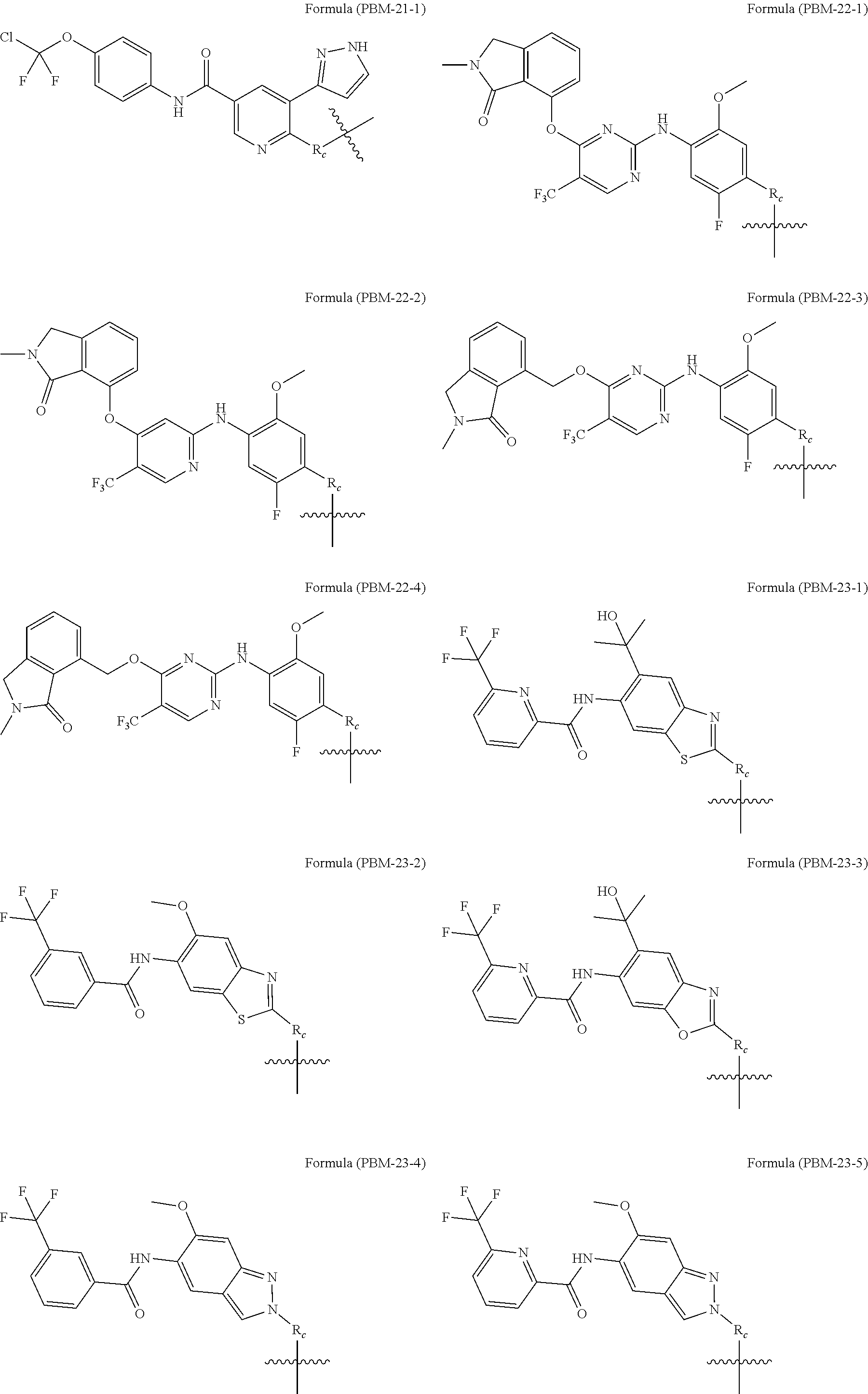 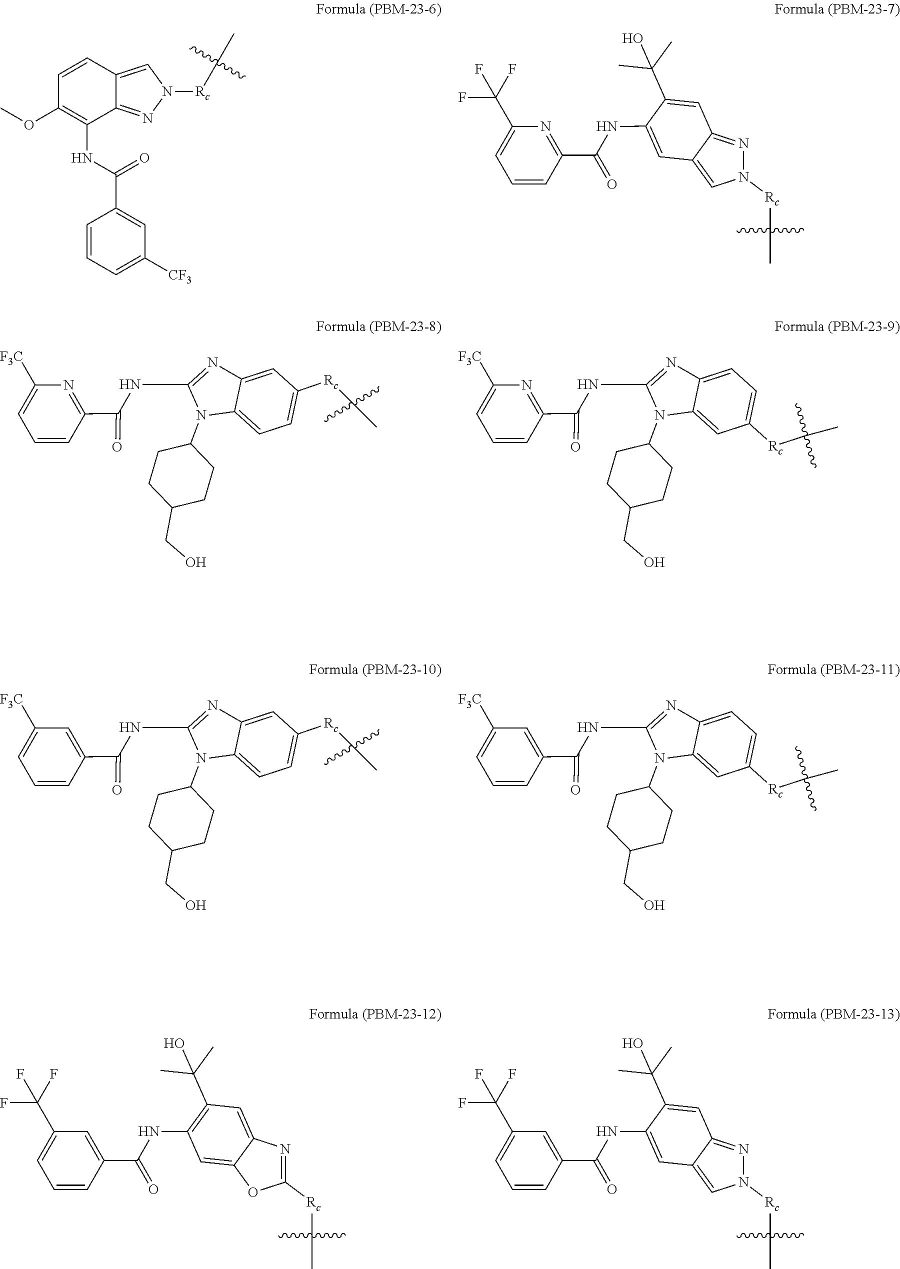 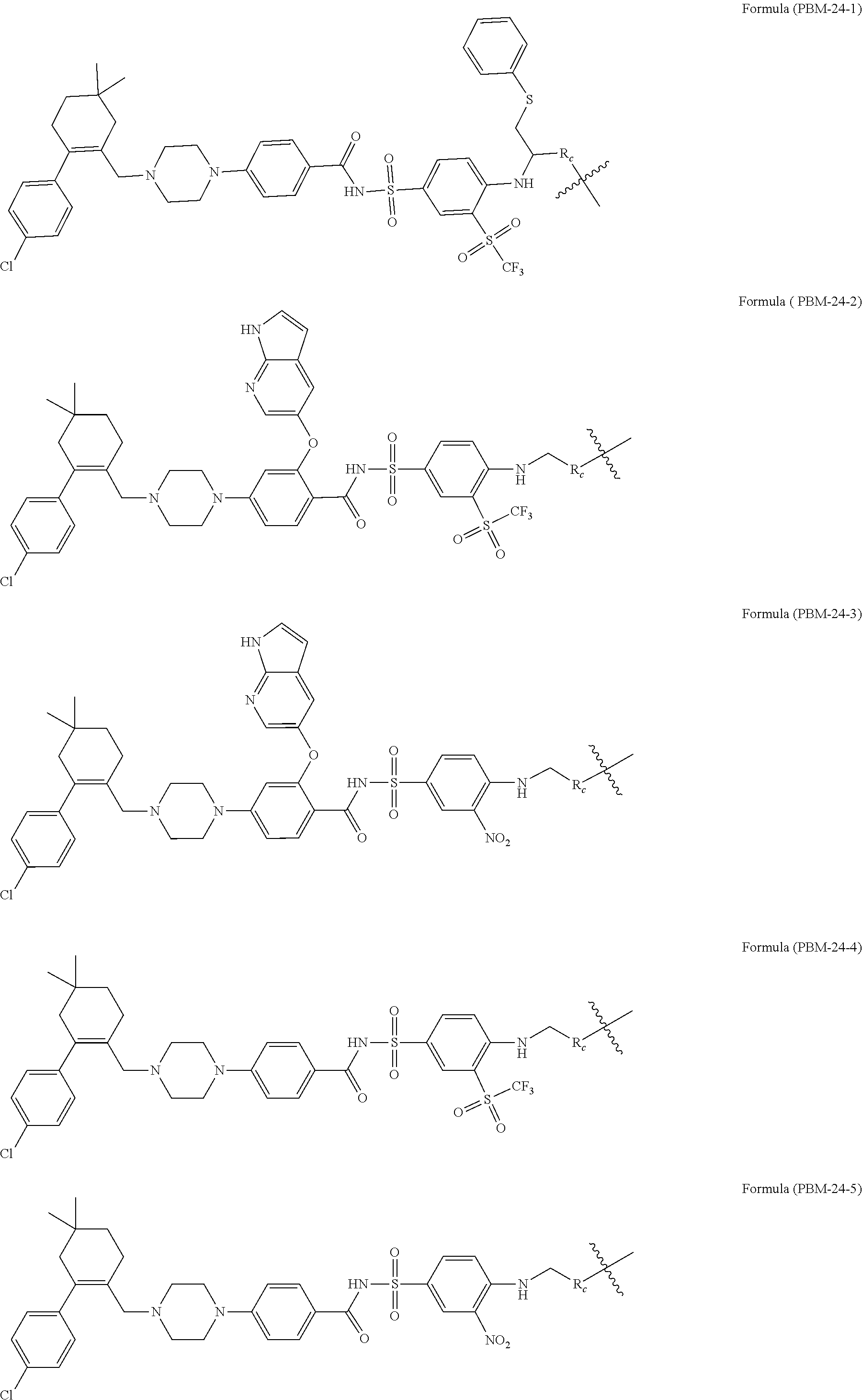 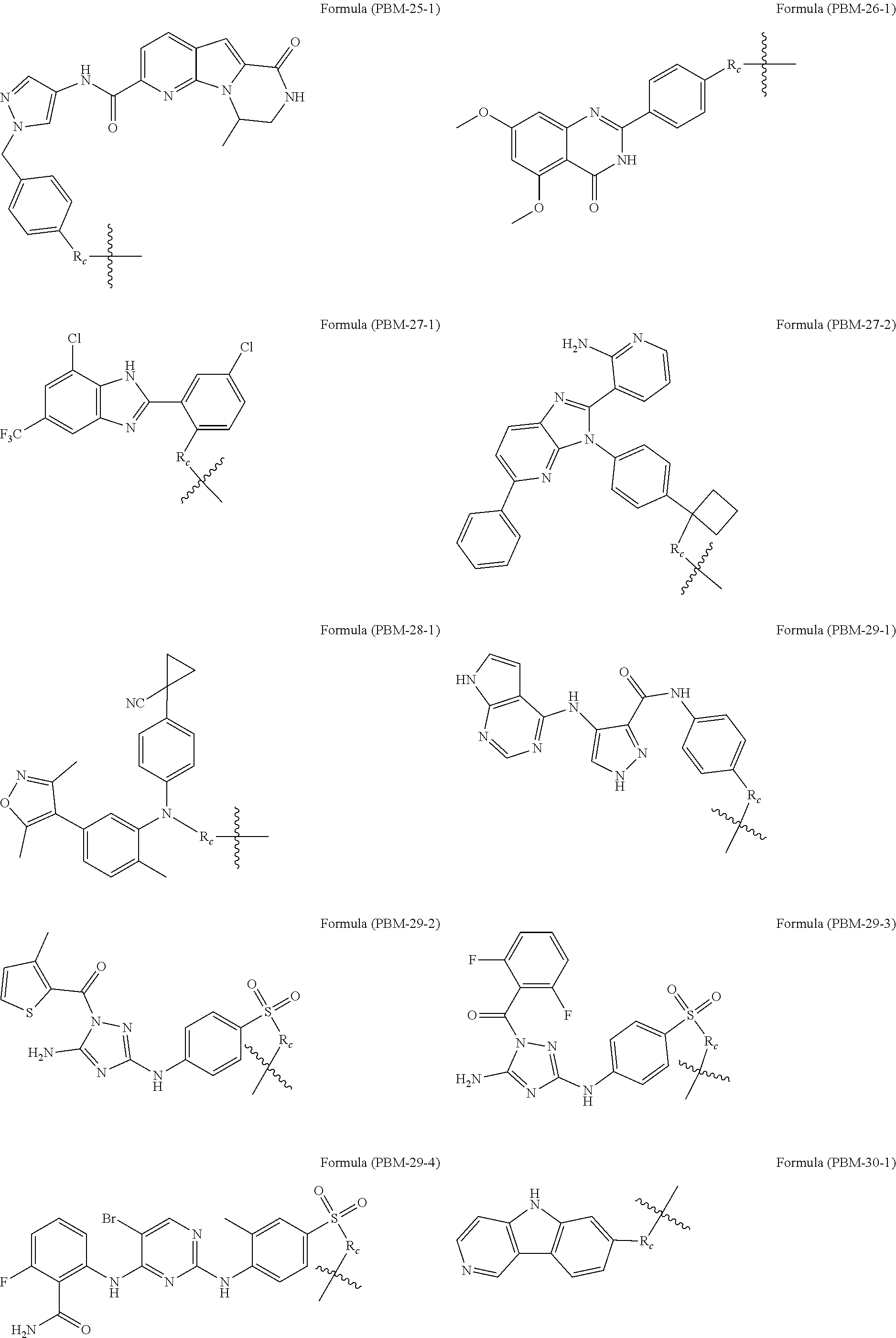 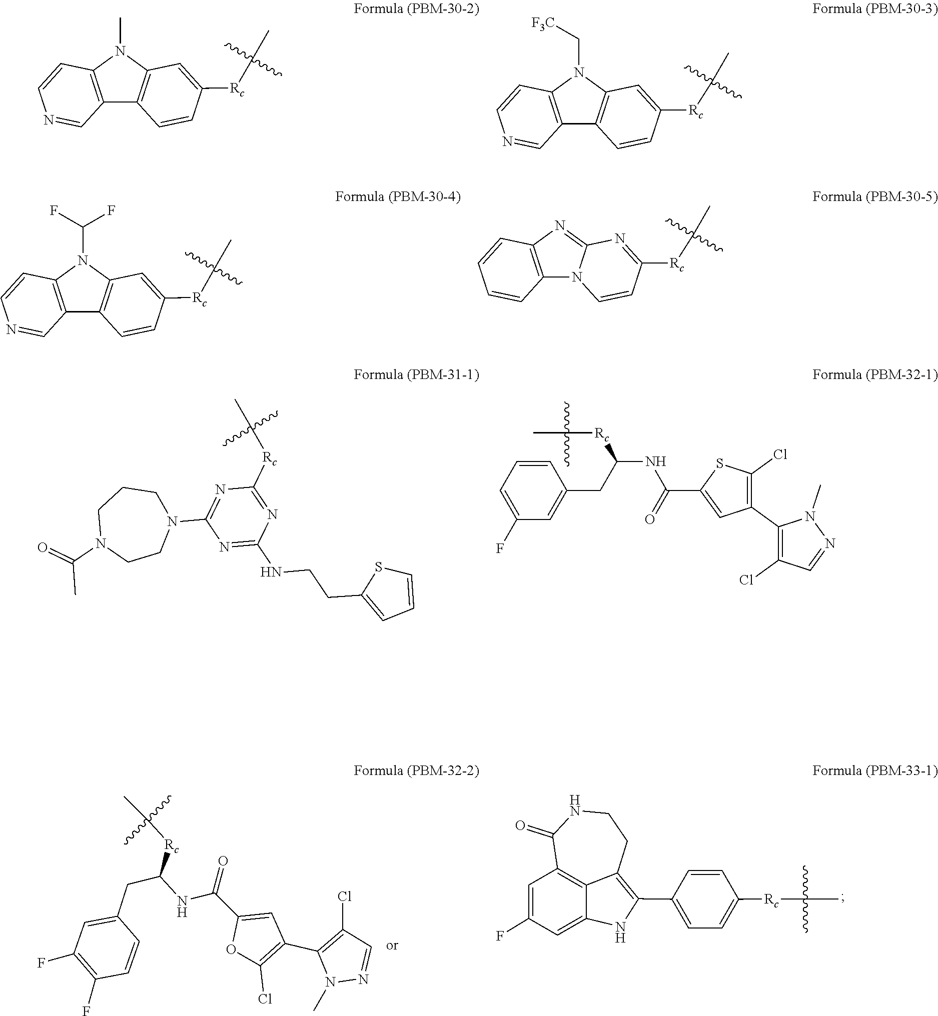
wherein R_{c} is as defined in claim 3.

5. The compound of Formula (I) or salts, enantiomers, stereoisomers, solvates, isotopically enriched analogs, prodrugs, or polymorphs thereof as claimed in claim 3, wherein
the R_{c} represents a bond, —O—, —NHC(O)—*, —C(O)NH—*, —C(O)O—*, —OC(O)—*, or —NH—, or
R_{c} represents a structure of the following formulae:
—N(R_{d})—, —N(R_{d})—R_{f}—N(Rₑ)—*, —R_{f}—C(O)NH—*, —R_{f}—NHC(O)—*, —R_{f}—C(O)O—*, —R_{f}—OC(O)—*, —R_{f}—, —R_{f}—N(R_{d})—*, —O—R_{f}—N(R_{d})—*, 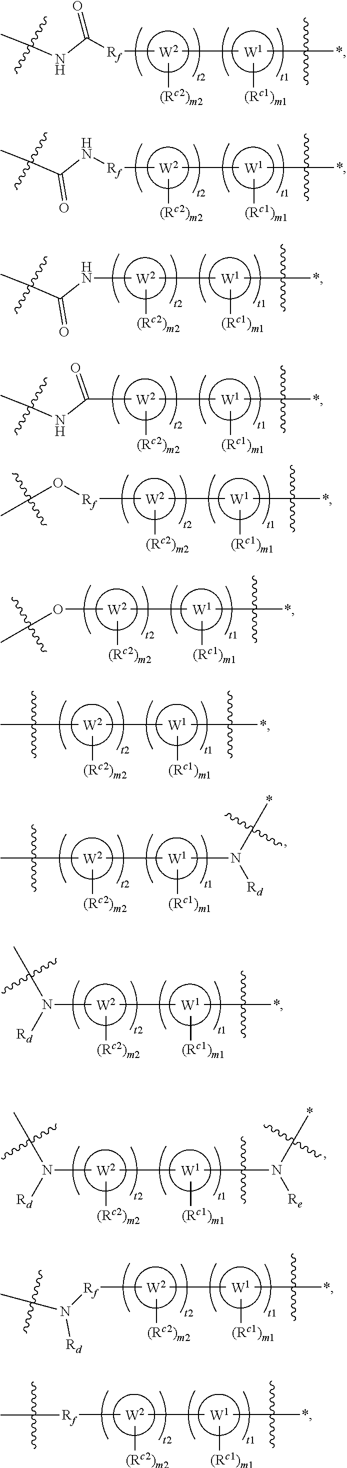 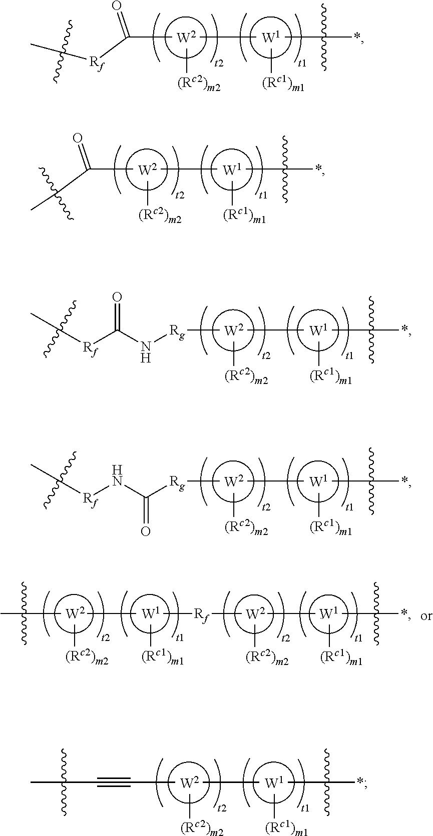
wherein each ring W¹ is the same or different and each independently represents 4-to 20-membered nitrogen-containing heterocyclylene, t1 represents an integer of 1 or 2, and (R_{c1})ₘ₁ indicates that each ring W¹ is independently optionally substituted with m1 R^{c1} groups, wherein each R^{c1} is independently deuterium, C₁₋₆ alkyl, deuterated C₁₋₆ alkyl, halogenated C₁₋₆ alkyl, C₁₋₆ alkoxy, halogenated C₁₋₆ alkoxy, deuterated C₁₋₆ alkoxy, halogen, amino, hydroxy, mercapto, cyano, carboxyl, C₂₋₆ alkynyl, C₂₋₆ alkenyl, optionally deuterated C₃₋₆ cycloalkyl, or R_{c4}—R_{c3}—, wherein R_{c3} is optionally substituted C₁₋₆ alkylene, or optionally substituted C₂₋₆ alkenylene, and R_{c4} represents halogen, R_{c5}R_{c6}N—, hydroxy, carboxyl, ethynyl, or vinyl, wherein R_{c5} and R_{c6} are the same or different and independently represent hydrogen or C₁₋₃ alkyl, and m1 represents an integer of 0-20;
each ring W² is the same or different and each independently represents 4-to 20-membered heterocyclylene, C₃₋₂₀ cycloalkylene, C₅₋₂₀ arylene, or 5-to 20-membered heteroarylene, t2 represents an integer of 0 or 1, and (R_{c2})ₘ₂ indicates that each ring W² is independently optionally substituted with m2 R_{c2} groups, wherein each R_{c2} is independently deuterium, C₁₋₆ alkyl, deuterated C₁₋₆ alkyl, halogenated C₁₋₆ alkyl, C₁₋₆ alkoxy, halogenated C₁₋₆ alkoxy, deuterated C₁₋₆ alkoxy, halogen, amino, hydroxy, mercapto, cyano, carboxyl, C₂₋₆ alkynyl, C₂₋₆ alkenyl, optionally deuterated C₃₋₆ cycloalkyl, or R_{c8}—R_{c7}—, wherein R_{c7} is optionally substituted C₁₋₆ alkylene or optionally substituted C₂₋₆ alkenylene, and R_{c8} represents halogen, R_{c9}R_{c10}N—, hydroxy, carboxyl, ethynyl, or vinyl, wherein R_{c9} and R_{c10} are the same or different and independently represent hydrogen or C₁₋₃ alkyl, and m2 represents an integer of 0-20; and
R_{d} and Rₑ each independently represent hydrogen, or C₁₋₆ alkyl; R_{f} and R_{g} each independently represent optionally substituted C₁₋₆ alkylene or optionally substituted C₂₋₆alkenylene;
the C₁₋₆ alkylene and C₂₋₆ alkenylene are each independently optionally substituted with one or more substituents selected from the group consisting of deuterium, halogen, hydroxy, mercapto, cyano, amino, nitro, C₁₋₆ alkyl, halogenated C₁₋₆ alkyl, deuterated C₁₋₆ alkyl, C₁₋₆ alkoxy, or halogenated C₁₋₆ alkoxy; and
symbol * indicates the point of attachment to LIN.

6. The compound of Formula (I) or salts, enantiomers, stereoisomers, solvates, isotopically enriched analogs, prodrugs, or polymorphs thereof as claimed in claim 3, wherein each ring W¹ is the same or different and each independently represents 4-to 15-membered nitrogen-containing heterocyclylene, e.g.,
piperidinylene, piperazinylene, morpholinylene, azetidinylene, pyrrolidinylene, imidazolidylene, pyrazolidylene, oxazolidinylene, thiazolidinylene, thiomorpholinylene, azepanylene, diazacycloheptanylene, azacyclooctylene, diazacyclooctylene, azabicyclo[3.1.1]heptanylene, azabicyclo[2.2.1]heptanylene, azabicyclo[3.2.1]octanylene, azabicyclo[2.2.2]octanylene, diazabicyclo[3.1.1]heptanylene, diazabicyclo[2.2.1]heptanylene, diazabicyclo[3.2.1]octanylene, diazabicyclo[2.2.2]octanylene, 3-azaspiro[5.5]undecanylene, 8-azaspiro[4.5]decanylene, 2-oxa-8-azaspiro[4.5]decanylene, or 3-methyl-2-oxa-8-azaspiro[4.5]decanylene, or 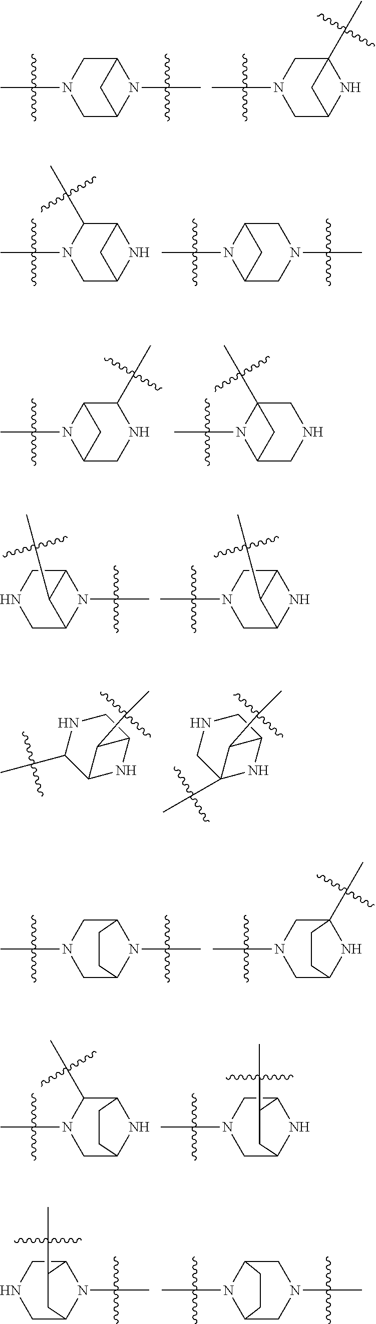 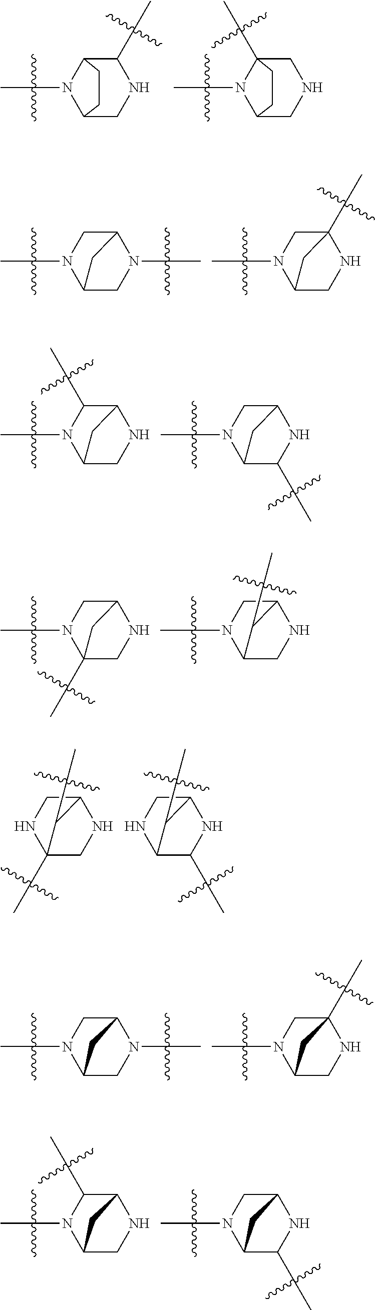 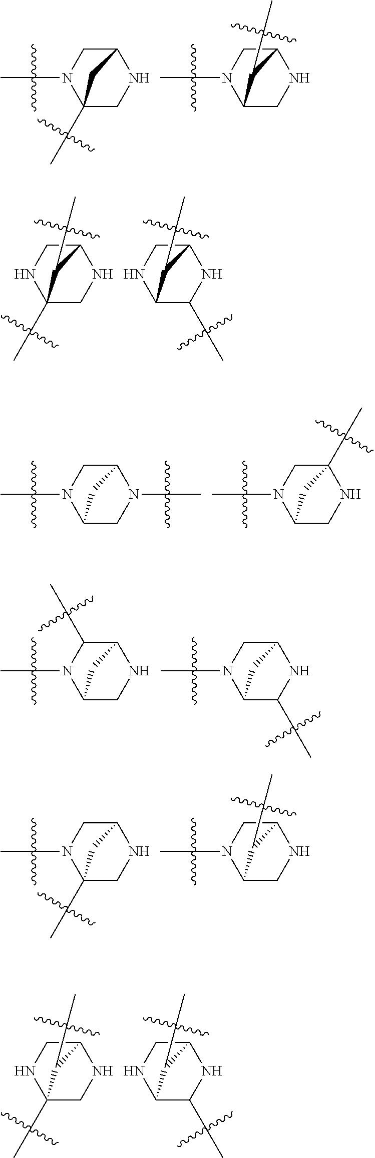 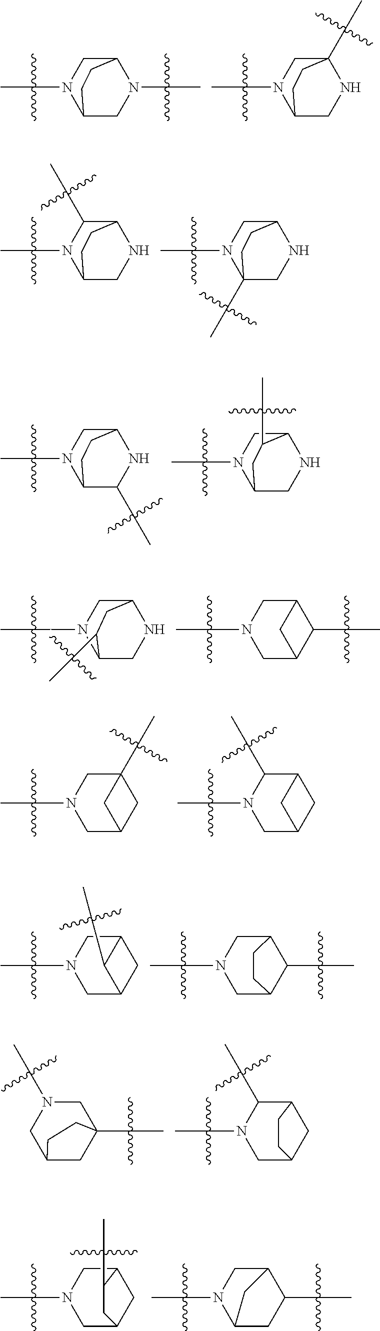 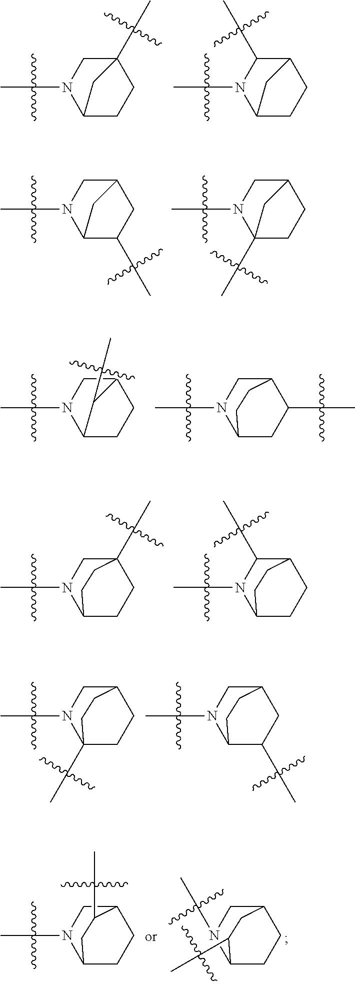
wherein each ring W¹ is each independently optionally substituted with m1 R^{c1} groups, wherein each R^{c1} is independently deuterium, C₁₋₆ alkyl, deuterated C₁₋₆ alkyl, halogenated C₁₋₆ alkyl, C₁₋₆ alkoxy, halogenated C₁₋₆ alkoxy, deuterated C₁₋₆ alkoxy, halogen, amino, hydroxy, mercapto, cyano, carboxyl, C₂₋₆ alkynyl, C₂₋₆ alkenyl, optionally deuterated C₃₋₆ cycloalkyl, or R_{c4}—R_{c3}—, wherein R^{c3} is optionally substituted C₁₋₆ alkylene or optionally substituted C₂₋₆ alkenylene, and R_{c4} represents halogen, R_{c5}R_{c6}N—, hydroxy, carboxyl, ethynyl, or vinyl, wherein R_{c5} and R_{c6} are the same or different and independently represent hydrogen, or C₁₋₃ alkyl, and the C₁₋₆ alkylene and the C₂₋₆ alkenylene are each independently optionally substituted with one or more substituents selected from the group consisting of deuterium, halogen, hydroxy, mercapto, cyano, amino, nitro, C₁₋₆ alkyl, halogenated C₁₋₆ alkyl, deuterated C₁₋₆ alkyl, C₁₋₆ alkoxy, or halogenated C₁₋₆ alkoxy, and m1 represents an integer of 0-20, and/or
each ring W² is the same or different and each independently represents 4-to 15-membered nitrogen-containing heterocyclylene, C₃₋₁₅ cycloalkylene, 6-to 15-membered arylene, or 5-to 15-memberedheteroarylene, e.g.,
piperidinylene, piperazinylene, morpholinylene, azetidinylene, pyrrolidinylene, imidazolidylene, pyrazolidylene, oxazolidinylene, thiazolidinylene, thiomorpholinylene, azepanylene, diazacycloheptanylene, azacyclooctylene, diazacyclooctylene, azabicyclo[3.1.1]heptanylene, azabicyclo[2.2.1]heptanylene, azabicyclo[3.2.1]octanylene, azabicyclo[2.2.2]octanylene, diazabicyclo[3.1.1]heptanylene, diazabicyclo[2.2.1]heptanylene, diazabicyclo[3.2.1]octanylene, diazabicyclo[2.2.2]octanylene, 3-azaspiro[5.5]undecanylene, 8-azaspiro[4.5]decanylene, 2-oxa-8-azaspiro[4.5]decanylene, 3-methyl-2-oxa-8-azaspiro[4.5]decanylene, cyclopropylene, cyclobutylene, cyclopentylene, cyclopentenylene, cyclohexylene, cyclohexenylene, cycloheptylene, cyclooctylene, decalinylene, octahydropentalenylene, octahydro-1H-indenylene, 2,3-dihydro-1H-indenylene, spiro-cycloalkylene, adamantanylene, noradamantanylene, norbornylene, phenylene, naphthylene, furanylene, oxazolylene, isoxazolylene, oxadiazolylene, thienylene, thiazolylene, isothiazolylene, thiadiazolylene, pyrrolylene, imidazolylene, pyrazolylene, triazolylene, pyridylene, pyrimidinylene, pyridazinylene, pyrazinylene, indolylene, isoindolylene, benzofuranylene, isobenzofuranylene, benzothienylene, indazolylene, benzimidazolylene, benzoxazolylene, benzisoxazolylene, benzothiazolylene, benzisothiazolylene, benzotriazolylene, benzo[2,1,3]oxadiazolylene, benzo[2,1,3]thiadiazolylene, benzo[1,2,3]thiadiazolylene, quinolinylene, isoquinolinylene, naphthyridinylene, cinnolinylene, quinazolinylene, quinoxalinylene, phthalazinylene, pyrazolo[1,5-a]pyridylene, pyrazolo[1,5-alpyrimidinylene, imidazo[1,2-a]pyridylene, 1H-pyrrolo[3,2-b]pyridylene, 1H-pyrrolo[2,3-b]pyridylene, pyrrolo[2,1-b]thiazolylene, imidazo[2,1-b]thiazolylene, or 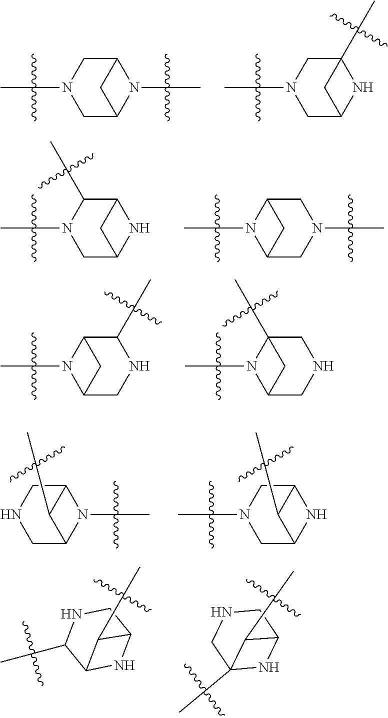 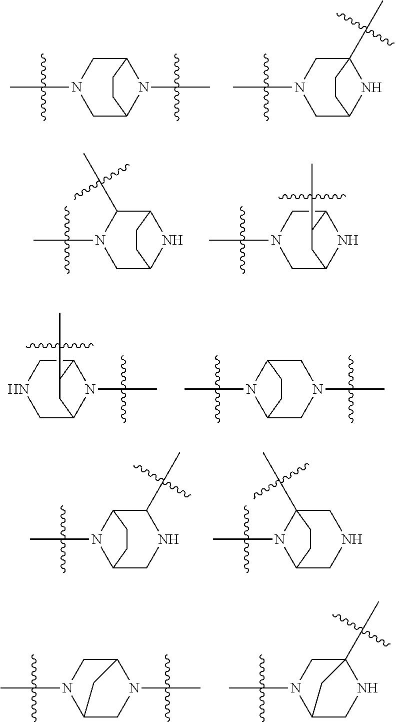 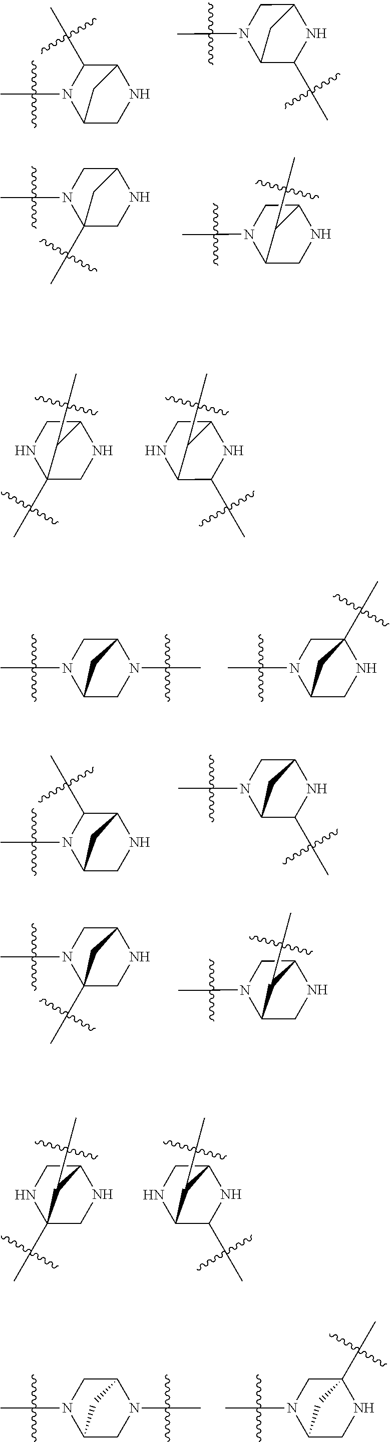 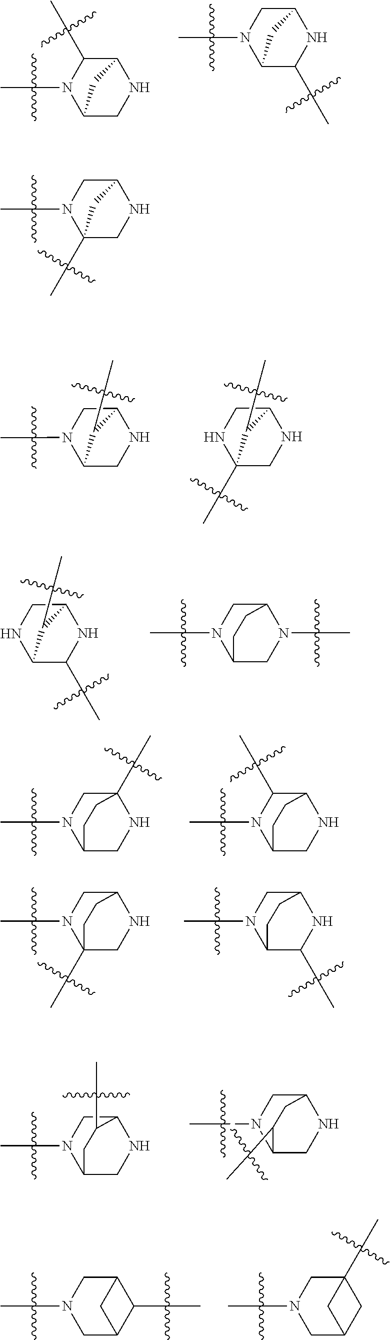 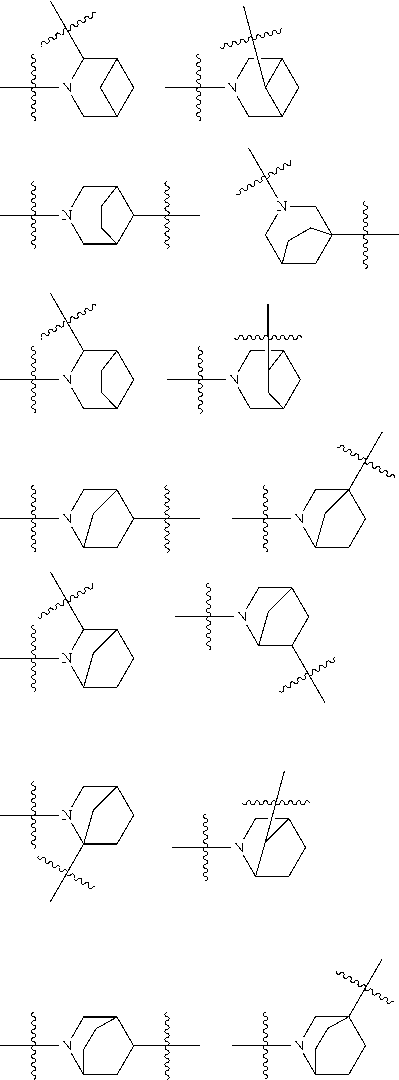 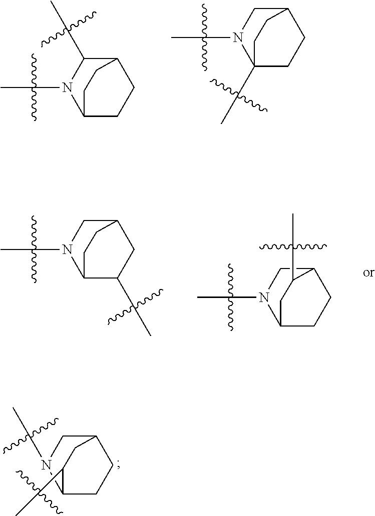
wherein each ring W² is each independently optionally substituted with m2 R_{c2} groups, wherein each R_{c2} is independently deuterium, C₁₋₆ alkyl, deuterated C₁₋₆ alkyl, halogenated C₁₋₆ alkyl, C₁₋₆ alkoxy, halogenated C₁₋₆ alkoxy, deuterated C₁₋₆ alkoxy, halogen, amino, hydroxy, mercapto, cyano, carboxyl, C₂₋₆ alkynyl, C₂₋₆ alkenyl, optionally deuterated C₃₋₆ cycloalkyl, or R_{c8}—R_{c7}—, wherein R_{c7} is optionally substituted C₁₋₆ alkylene or optionally substituted C₂₋₆ alkenylene, and R_{c8} represents halogen, R_{c9}R_{c10}N—, hydroxy, carboxyl, ethynyl, or vinyl, wherein R_{c9} and R_{c10} are the same or different and independently represent hydrogen or C₁₋₃ alkyl, and the C₁₋₆ alkylene and C₂₋₆ alkenylene are each independently optionally substituted with one or more substituents selected from the group consisting of deuterium, halogen, hydroxy, mercapto, cyano, amino, nitro, C₁₋₆ alkyl, halogenated C₁₋₆ alkyl, deuterated C₁₋₆ alkyl, C₁₋₆ alkoxy, or halogenated C₁₋₆ alkoxy, and m2 represents an integer of 0-20.

7. The compound of Formula (I) or salts, enantiomers, stereoisomers, solvates, isotopically enriched analogs, prodrugs, or polymorphs thereof as claimed in claim 3, wherein the 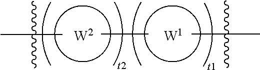
moiety of R_{c} in each general formula represents the following groups: 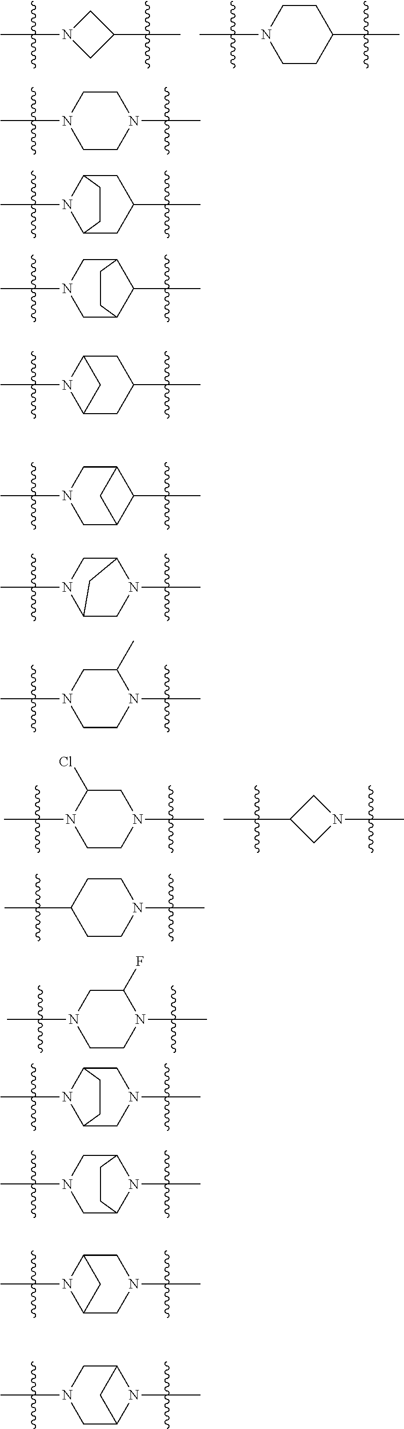 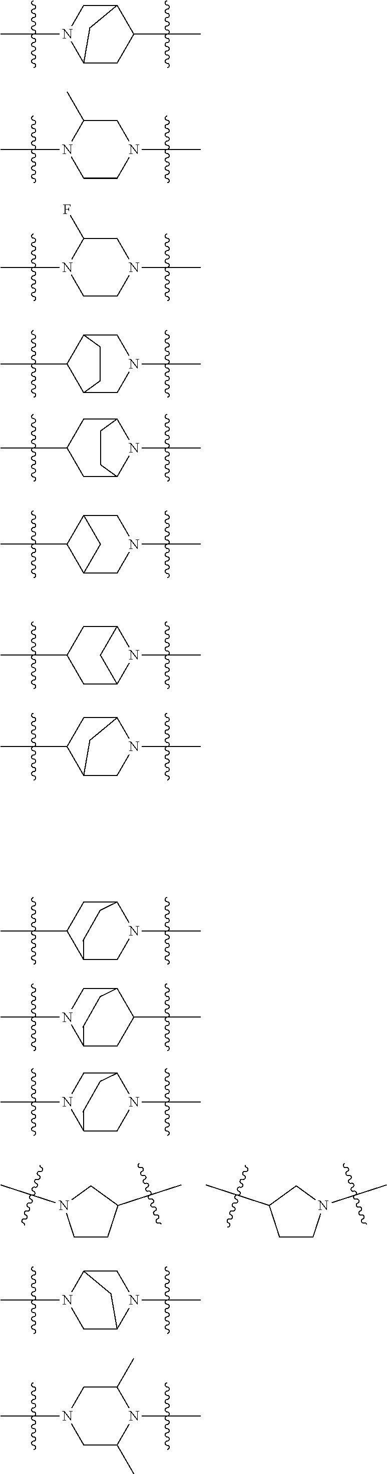 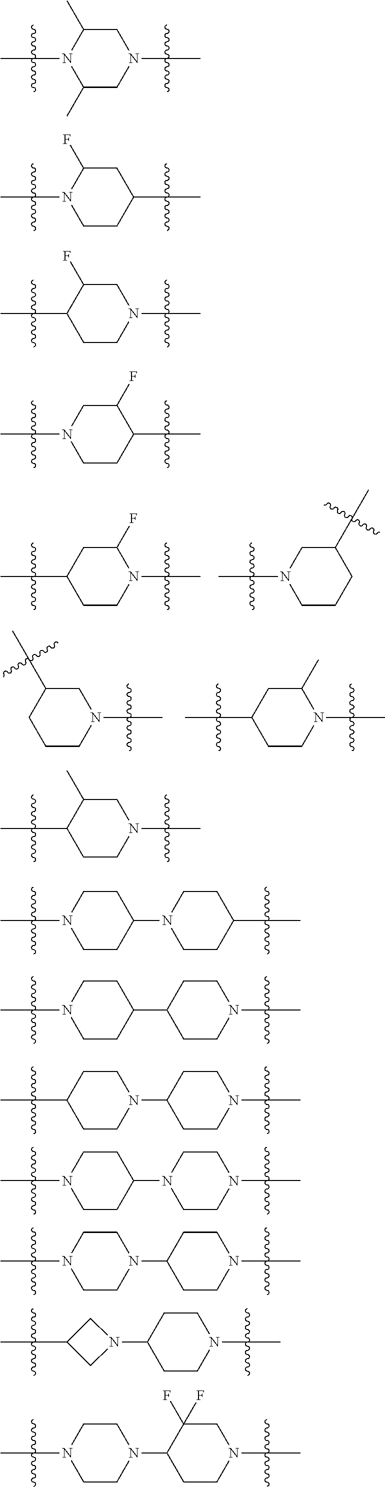 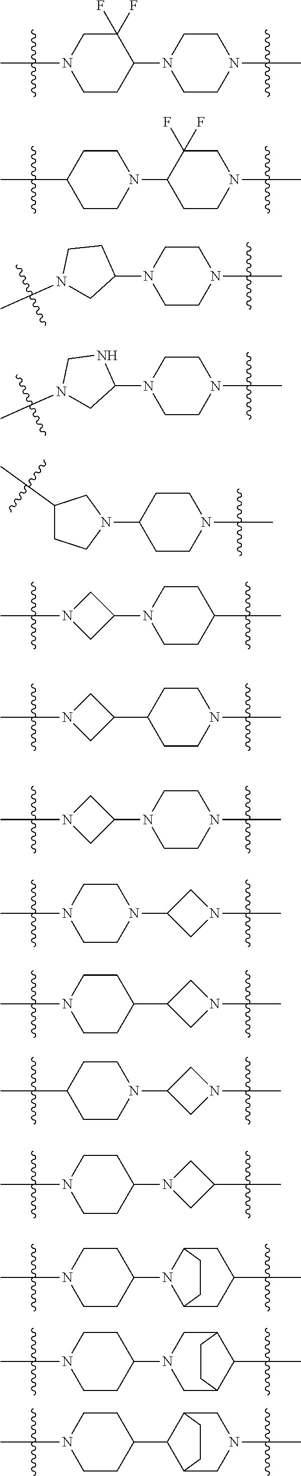 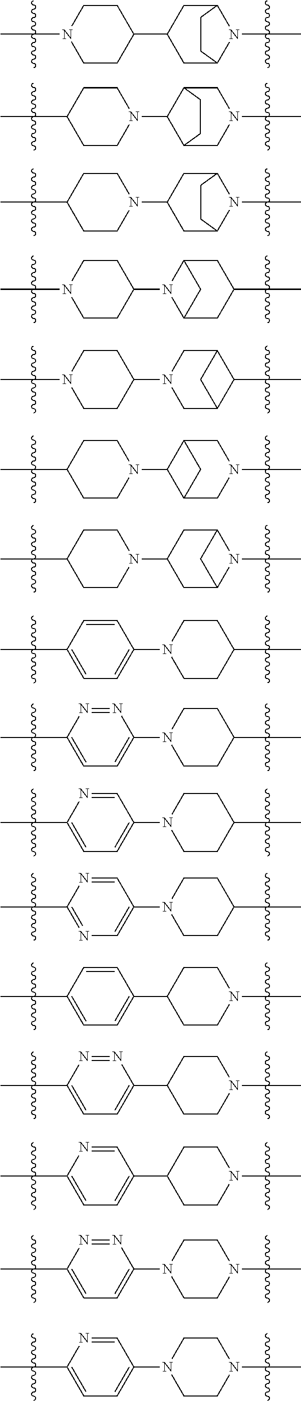 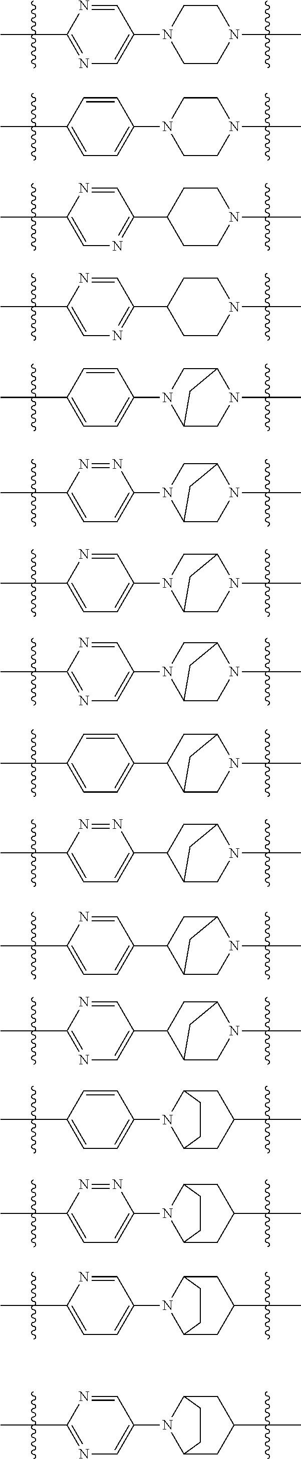 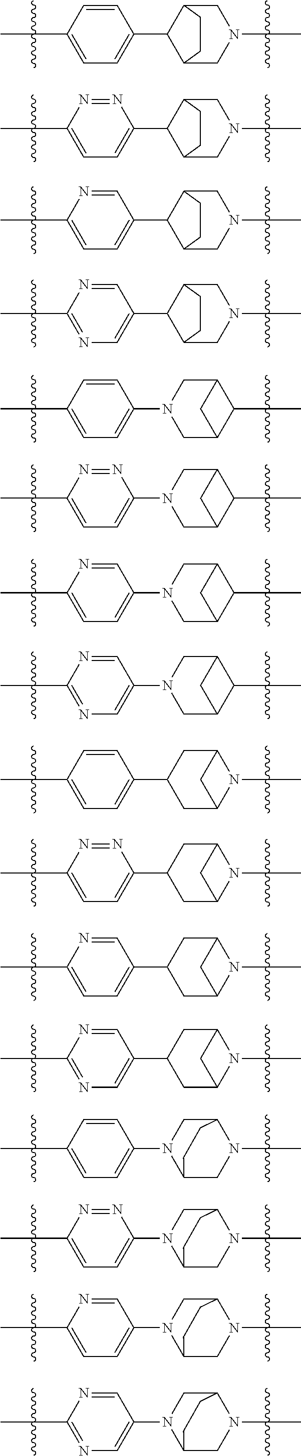 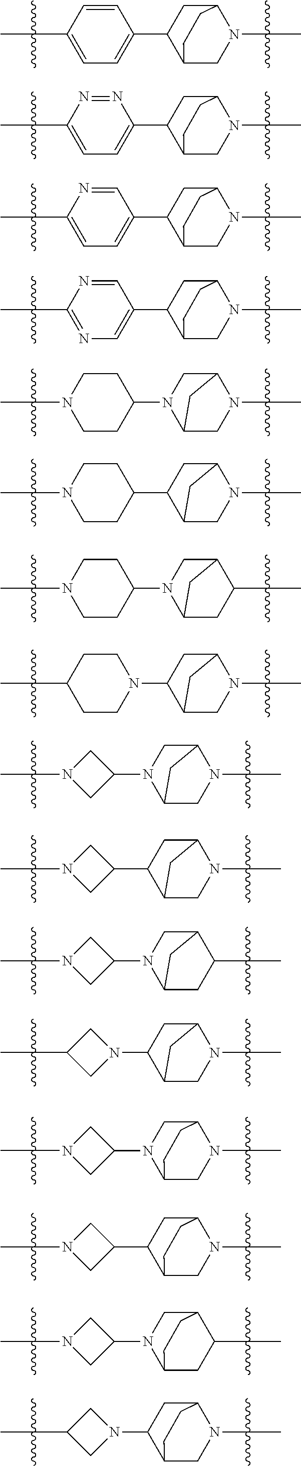 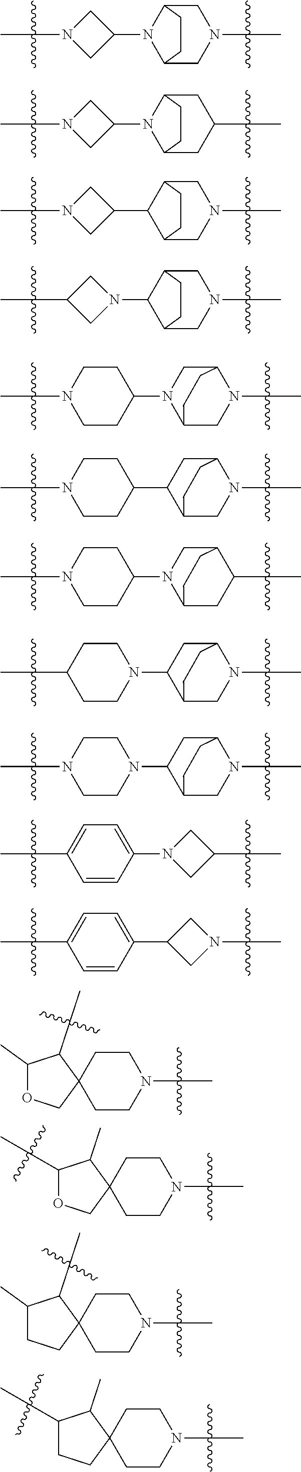 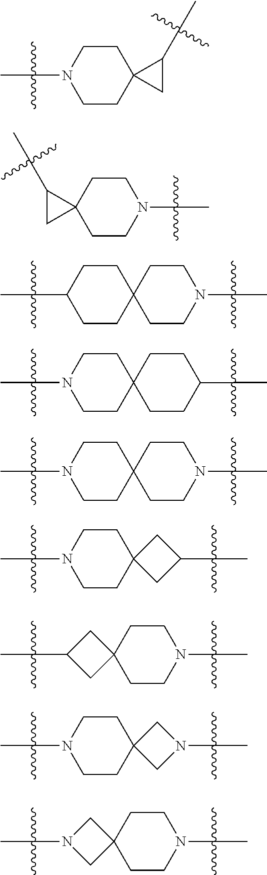 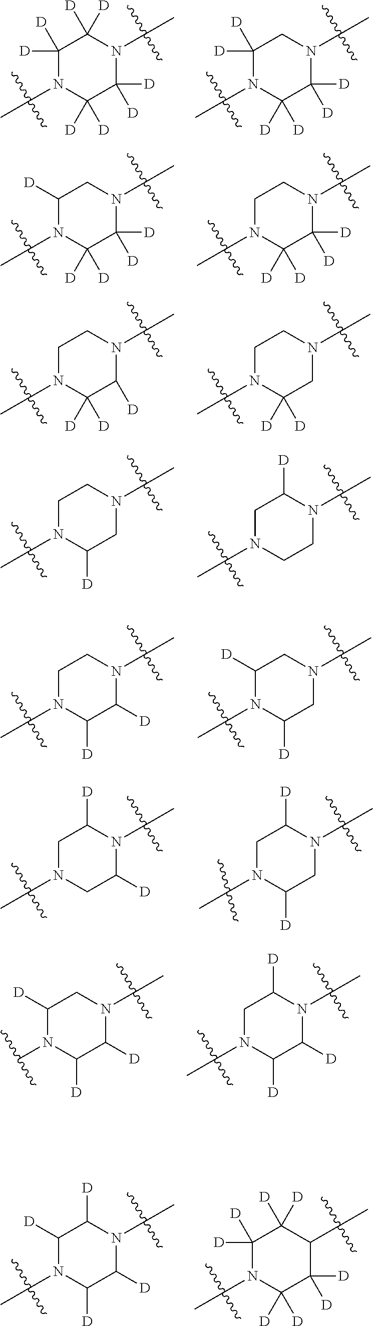 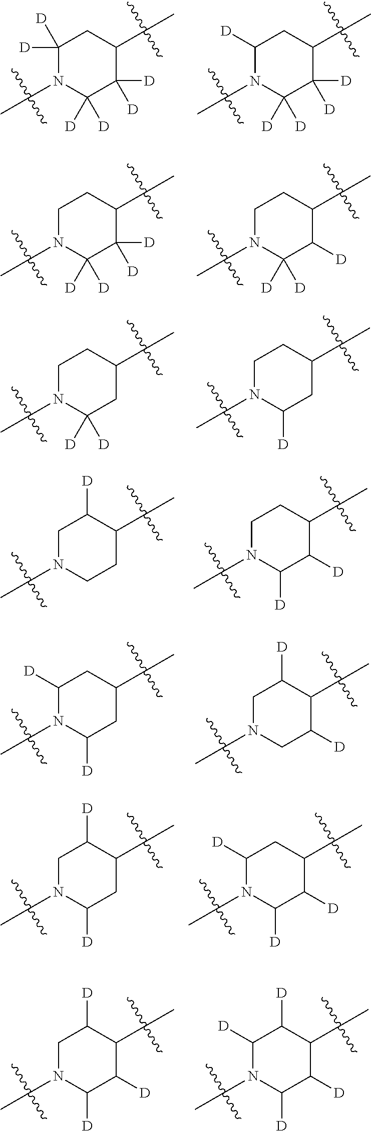 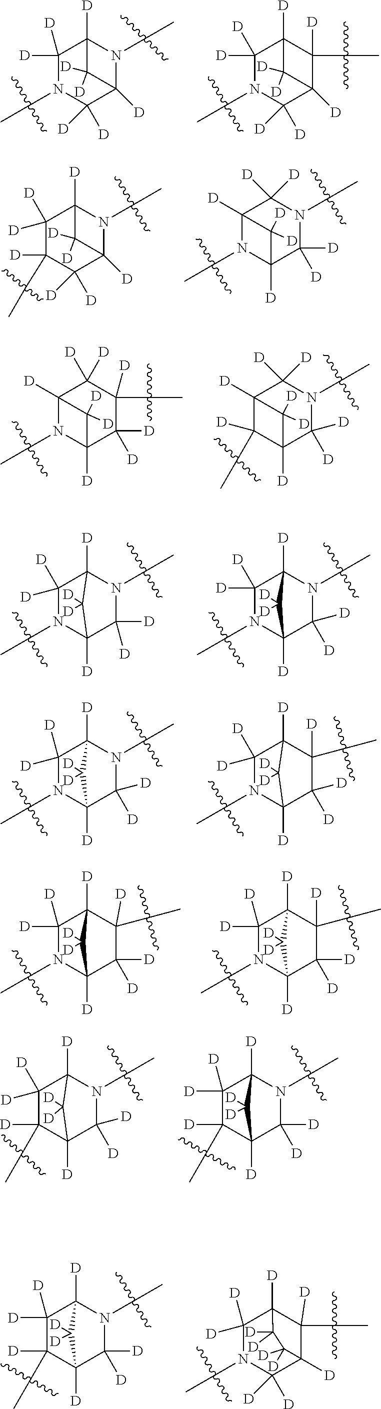 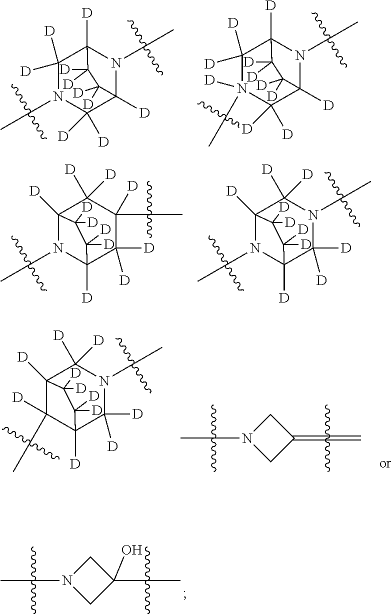
wherein the groups are each independently optionally substituted with one or more substituents selected from the group consisting of deuterium, optionally deuterated C₁₋₆ alkyl, optionally halogenated C₁₋₆ alkyl, C₁₋₆ alkoxy, halogenated C₁₋₆ alkoxy, deuterated C₁₋₆ alkoxy, halogen, amino, hydroxy, mercapto, cyano, carboxyl, C₂₋₆ alkynyl, C₂₋₆ alkenyl, optionally deuterated C₃₋₆ cycloalkyl, R_{c12}—R_{c11}, and any combination thereof, wherein R^{c11} is optionally substituted C₁₋₆ alkylene or optionally substituted C₂₋₆ alkenylene, and R_{c12} represents halogen, R_{c13}R_{c14}N—, hydroxy, carboxyl, ethynyl, or vinyl, wherein R_{c13} and R_{c14} are the same or different and independently represent hydrogen, or C₁₋₃ alkyl, and the C₁₋₆ alkylene and C₂₋₆ alkenylene are each independently optionally substituted with one or more substituents selected from the group consisting of deuterium, halogen, hydroxy, mercapto, cyano, amino, nitro, C₁₋₆ alkyl, halogenated C₁₋₆ alkyl, deuterated C₁₋₆ alkyl, C₁₋₆ alkoxy, or halogenated C₁₋₆ alkoxy.

8. The compound of Formula (I) or salts, enantiomers, stereoisomers, solvates, isotopically enriched analogs, prodrugs, or polymorphs thereof as claimed in claim 3, wherein the R_{c} represents the following groups:
a bond, —O—, —NHC(O)—*, —C(O)NH—*, —C(O)O—*, —OC(O)—*, —CH₂C(O)O—*, —CH₂OC(O)—*, —NH—, —N(CH₃)—, —N(CH₃)—(CH₂)₂—N(CH₃)—*, —N(CH₃)—(CH₂)₃—N(CH₃)—*, —NH—(CH₂)₂—N(CH₃)—*, —N(CH₃)—(CH₂)₃—NH—*, —CH₂—NHC(O)—*, —CH₂—C(O)NH—*, —CH₂—NH—*, —O—(CH₂)₂—N(CH₃)—*, —O—(CH₂)₂—NH—*, or —CH₂—N(CH₃)—*, or 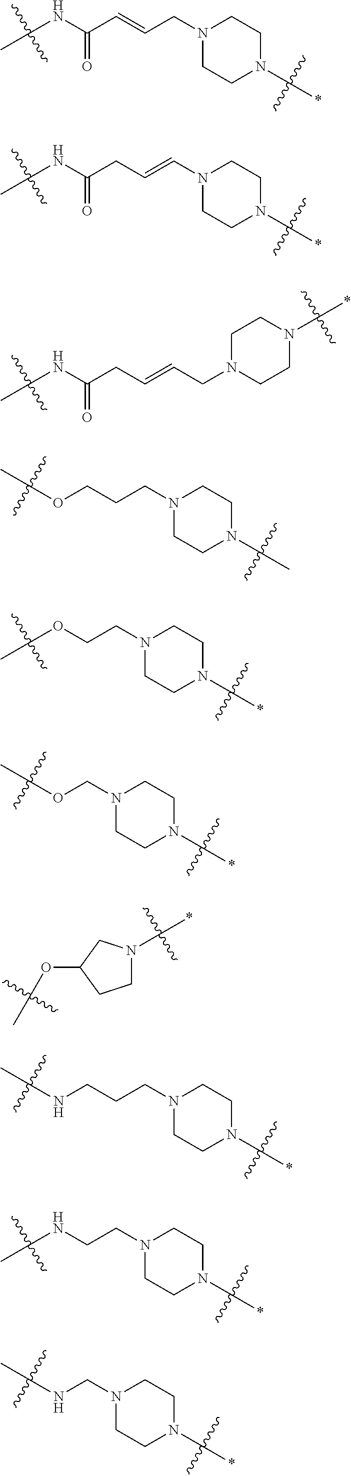 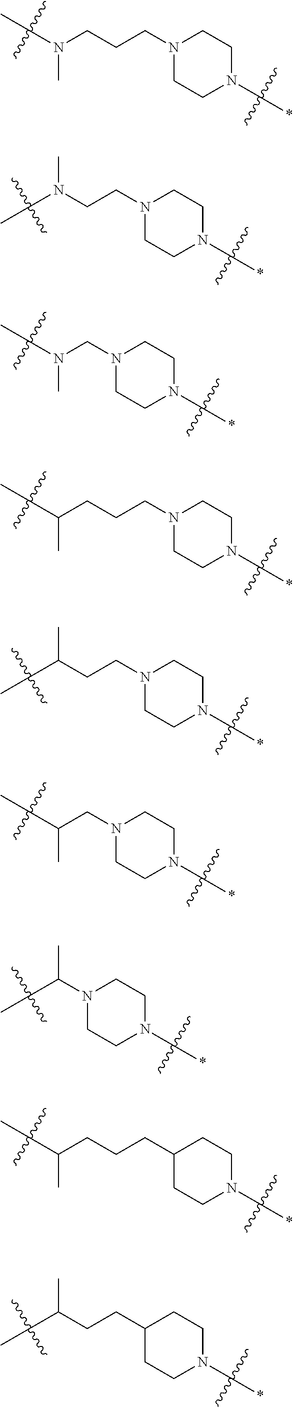 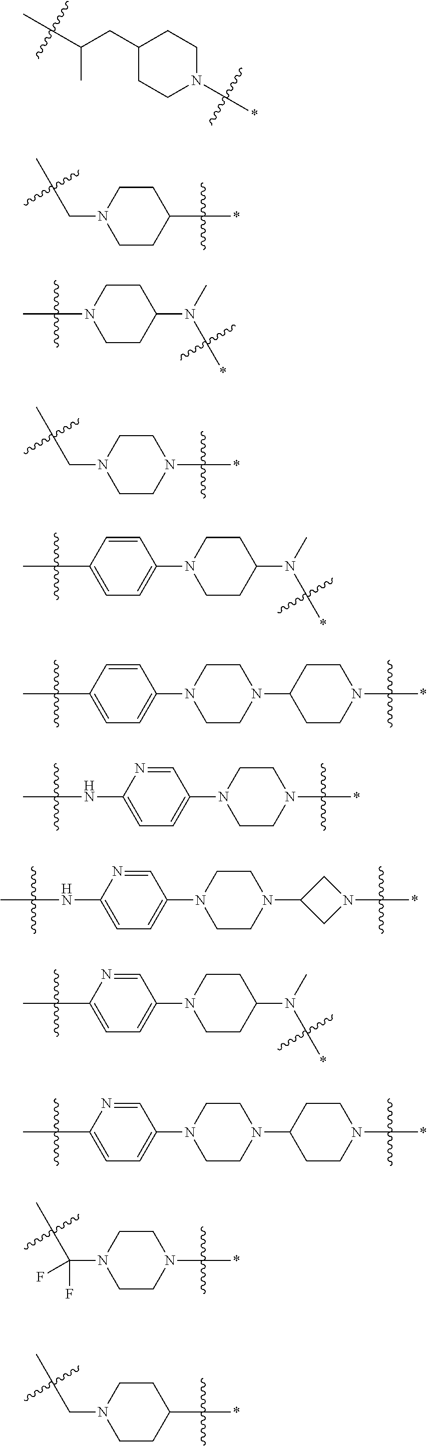 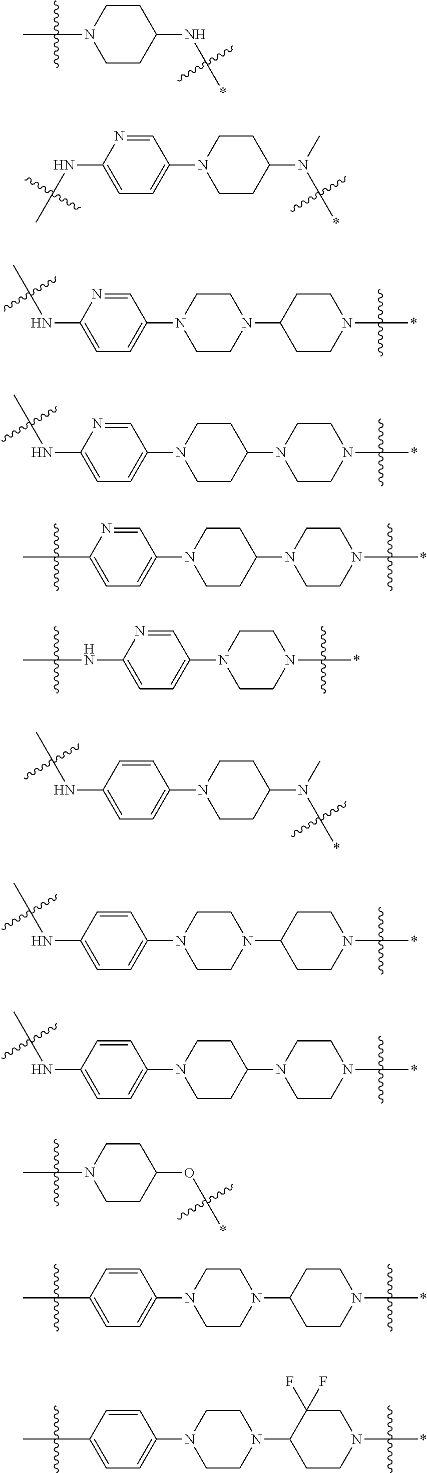 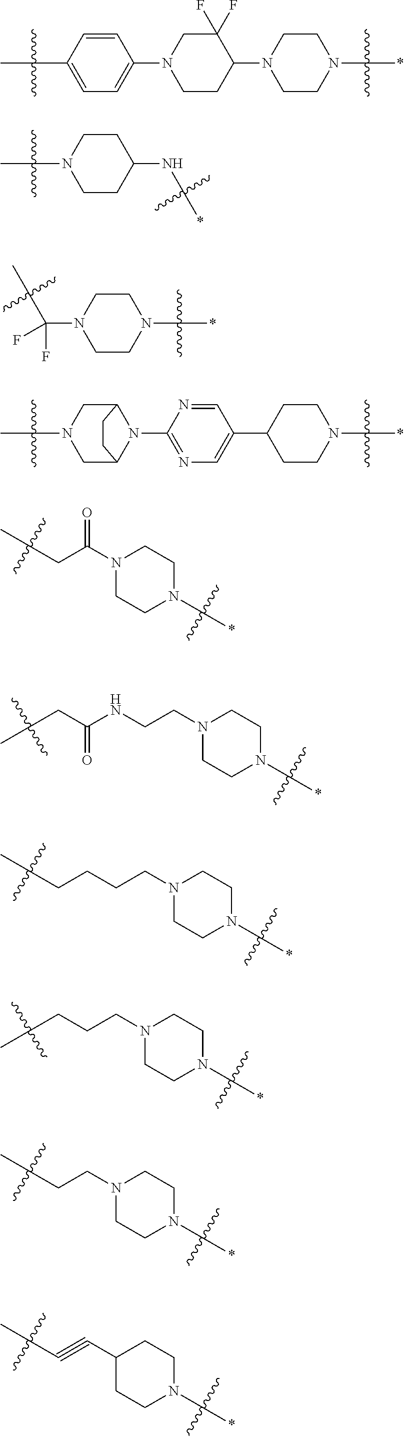 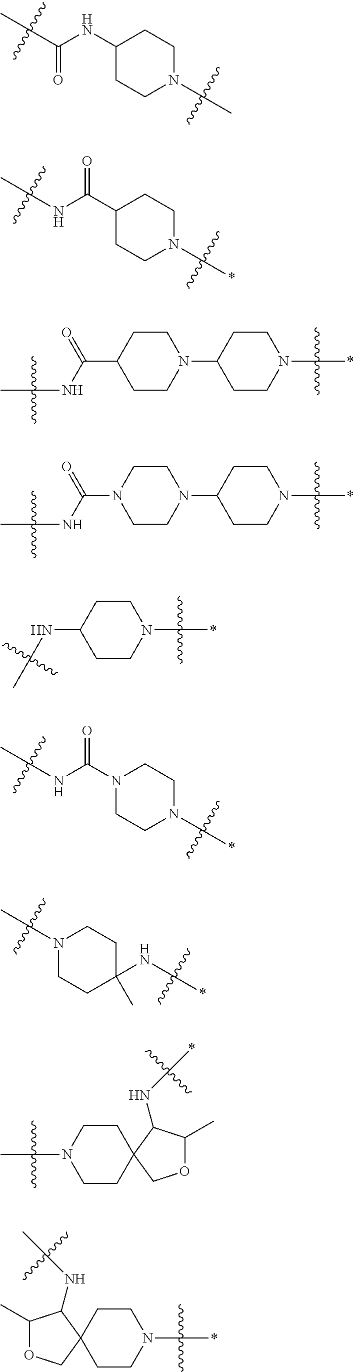 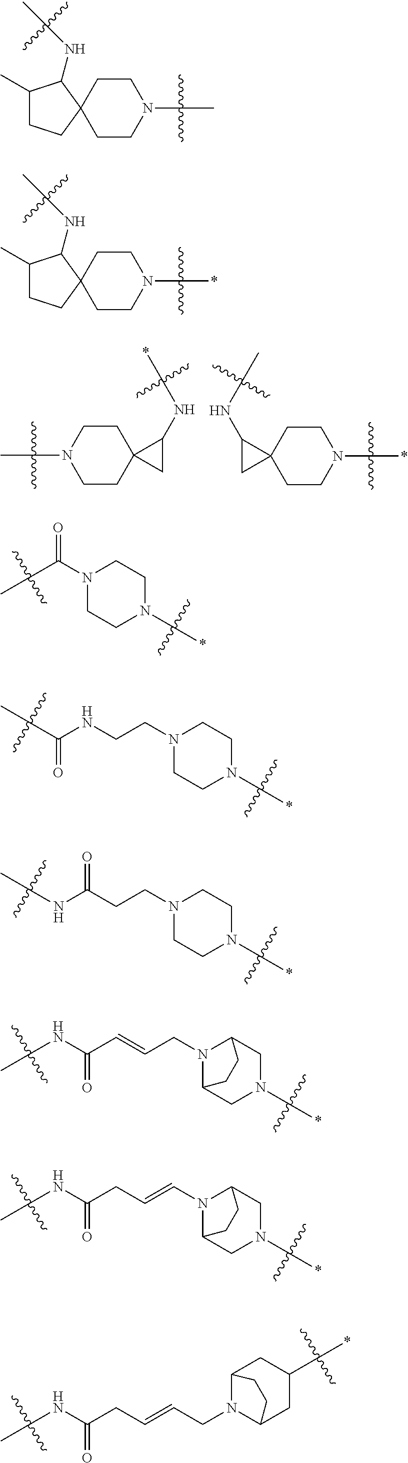 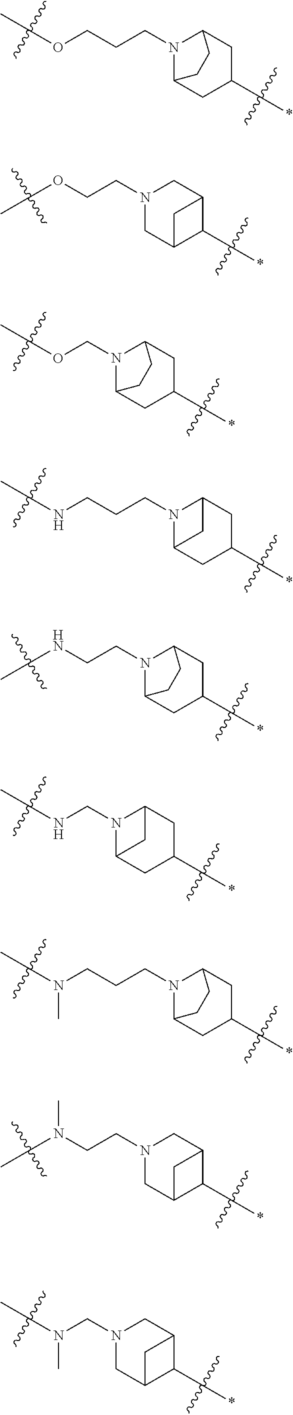 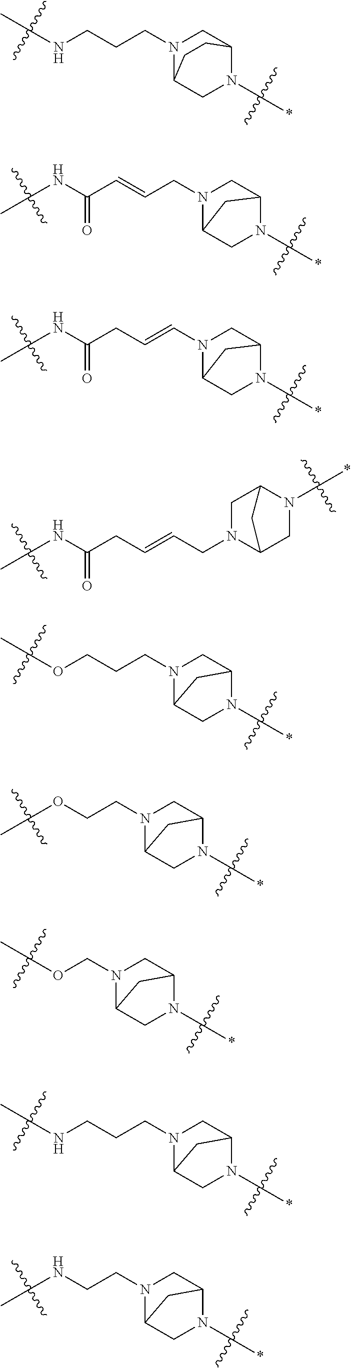 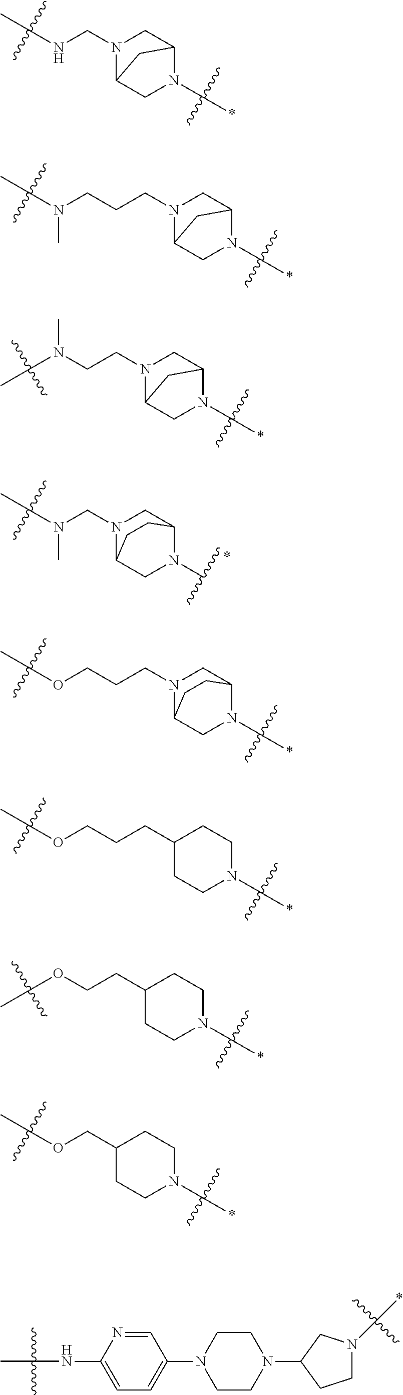 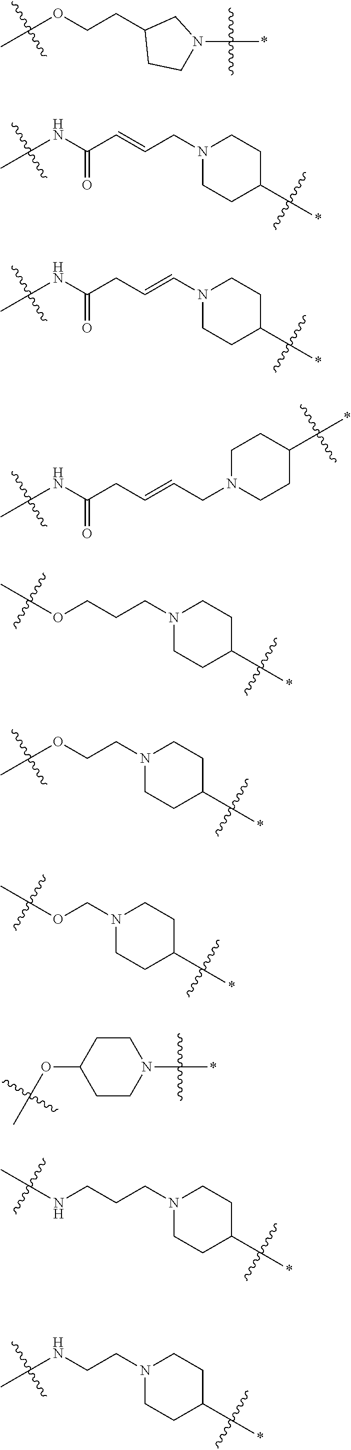 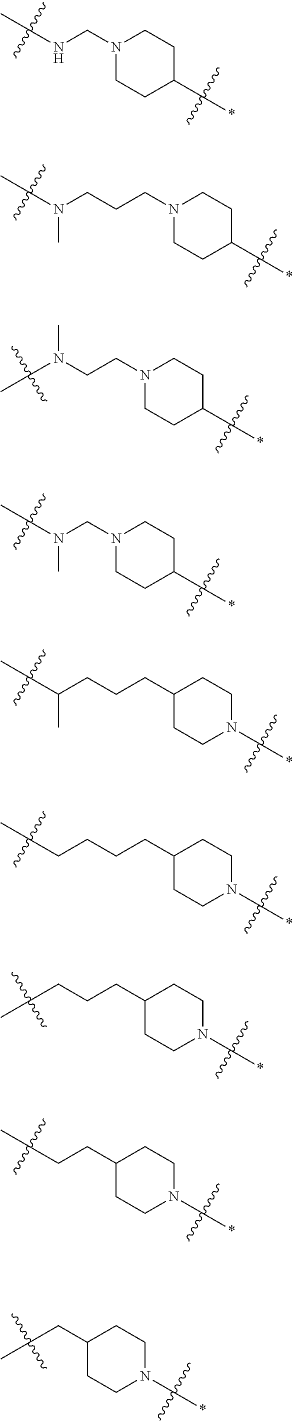 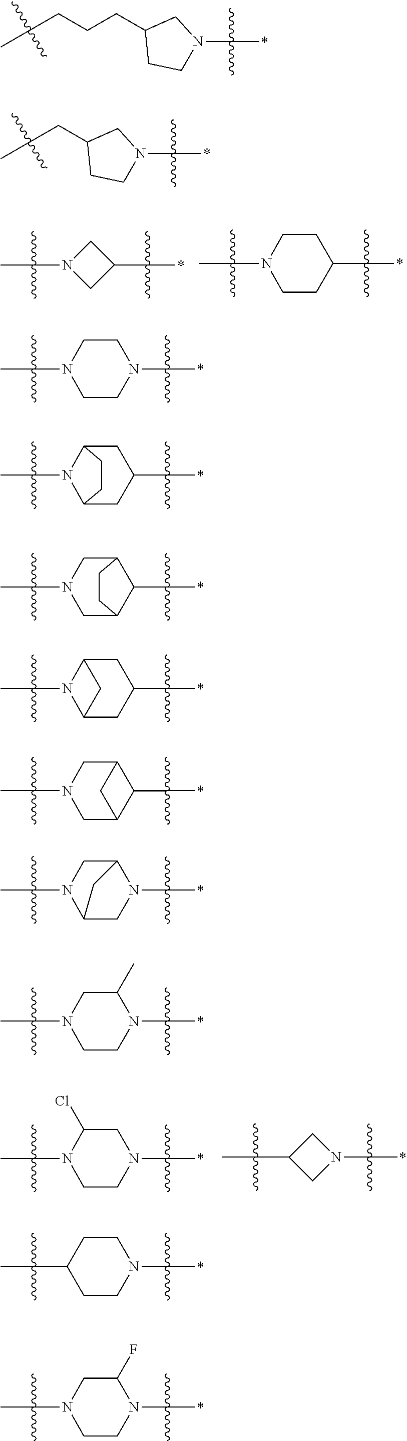 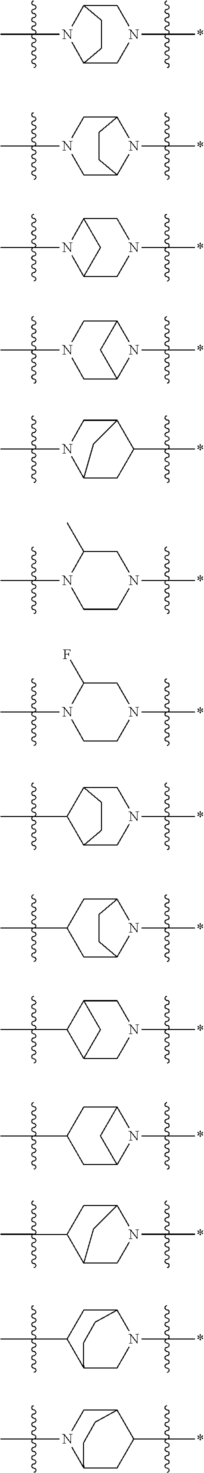 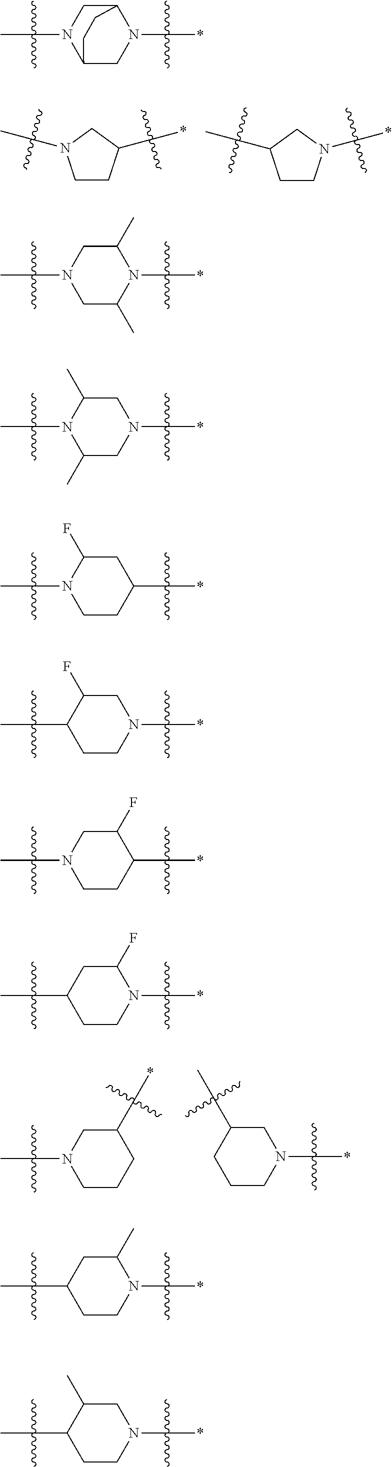 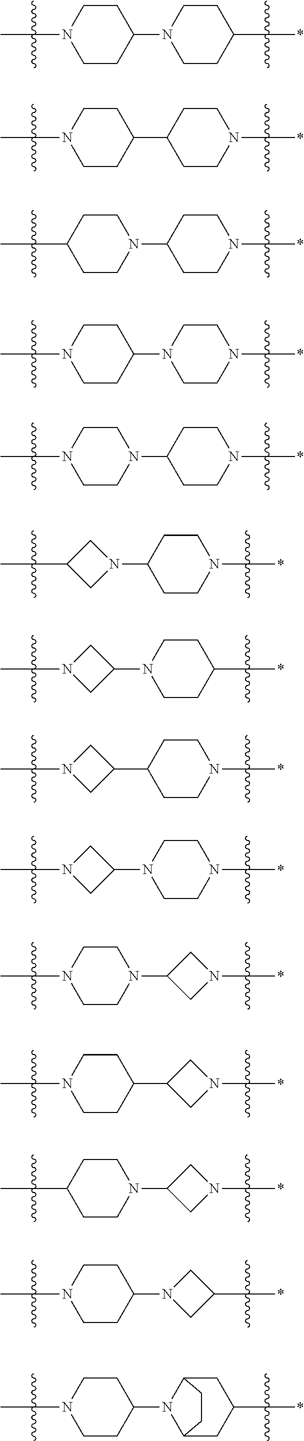 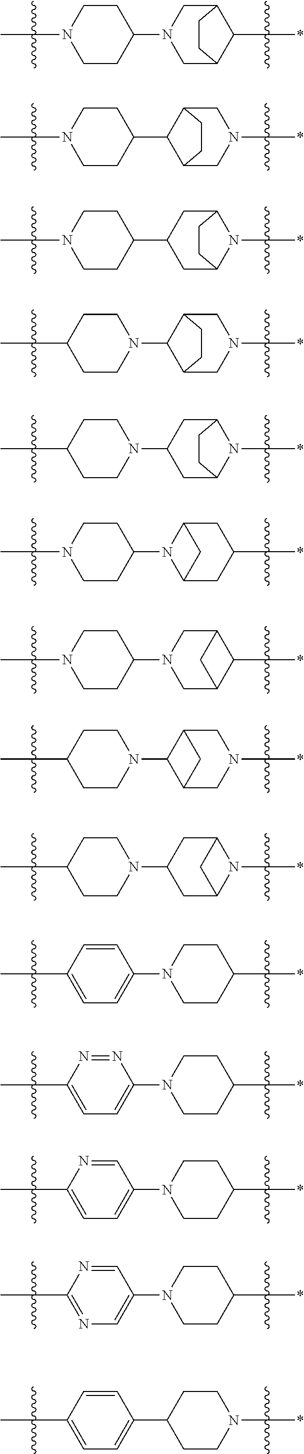 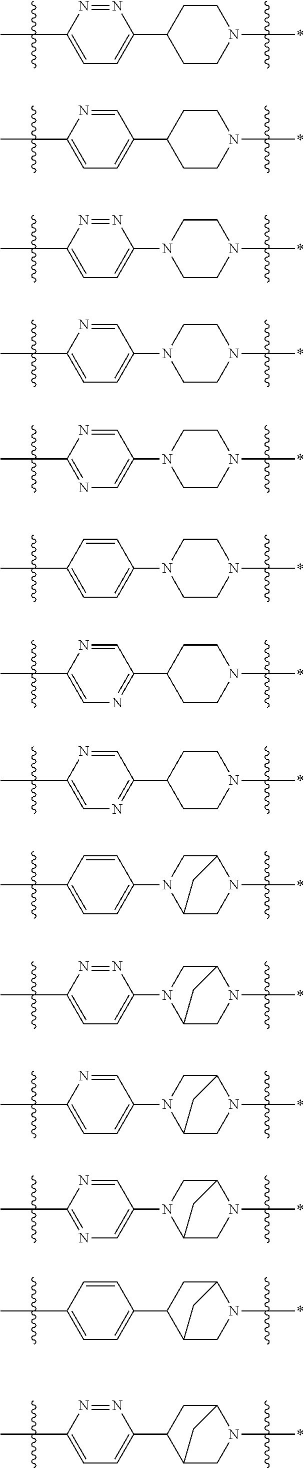 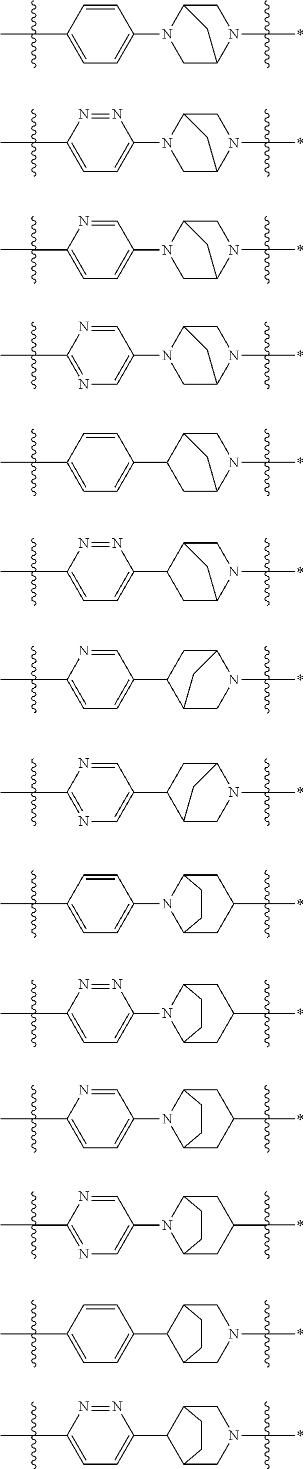 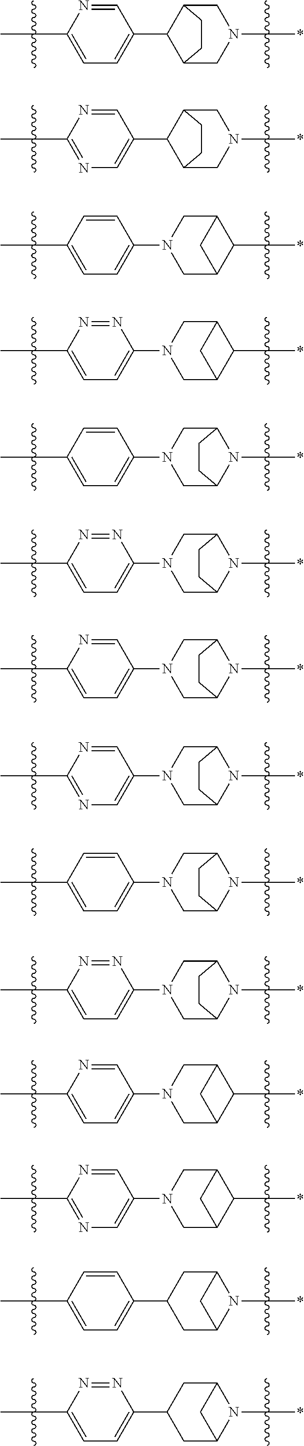 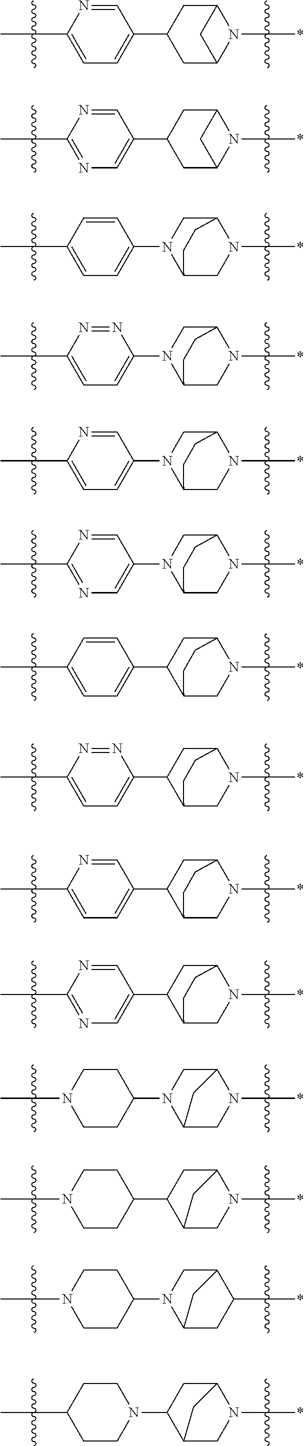 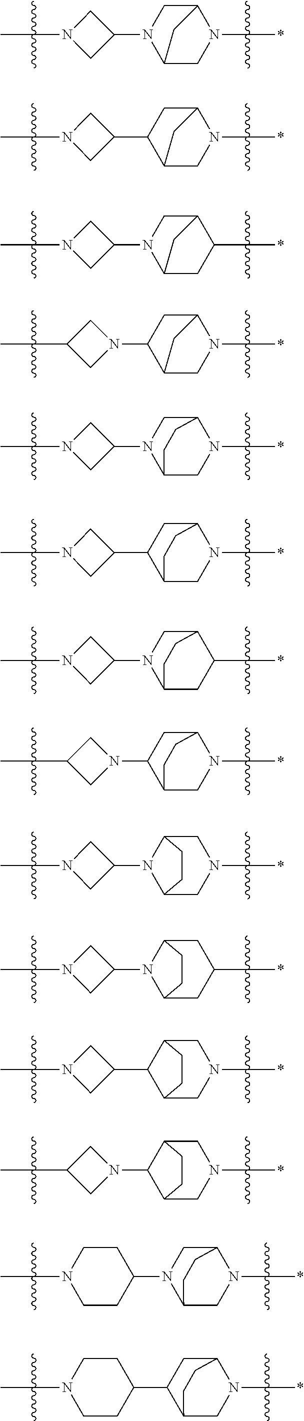 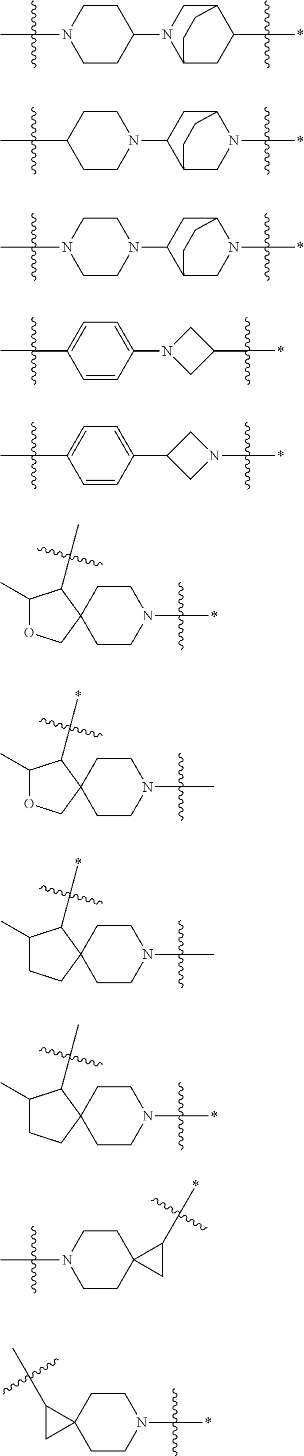 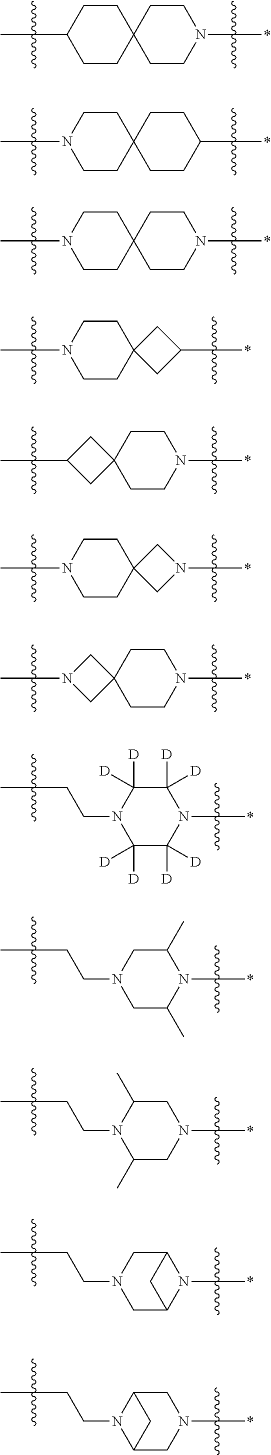 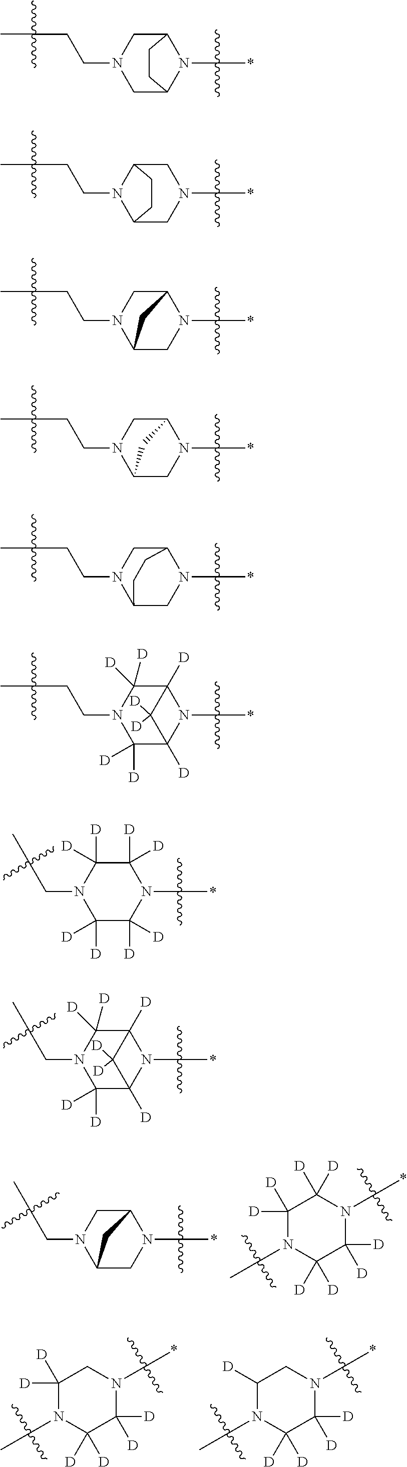 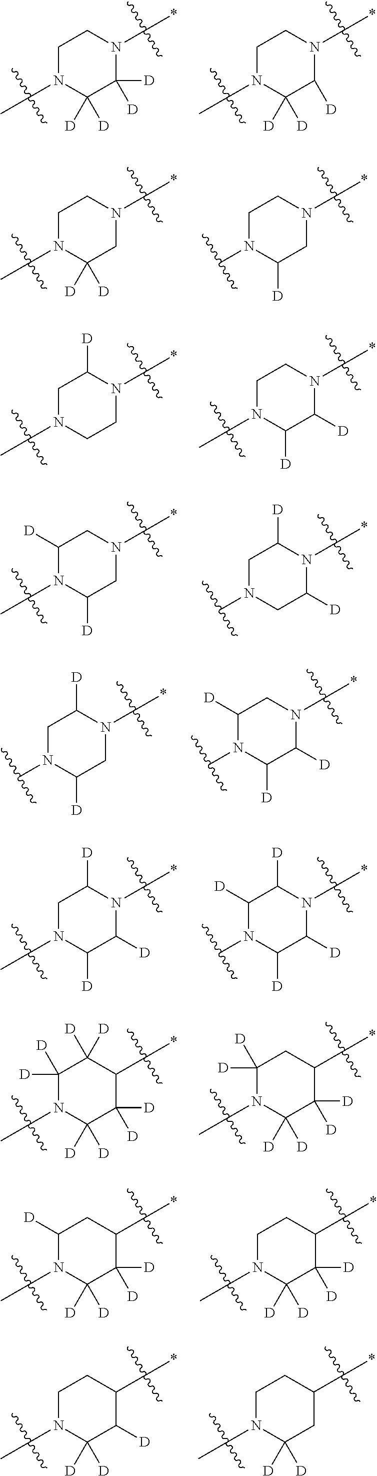 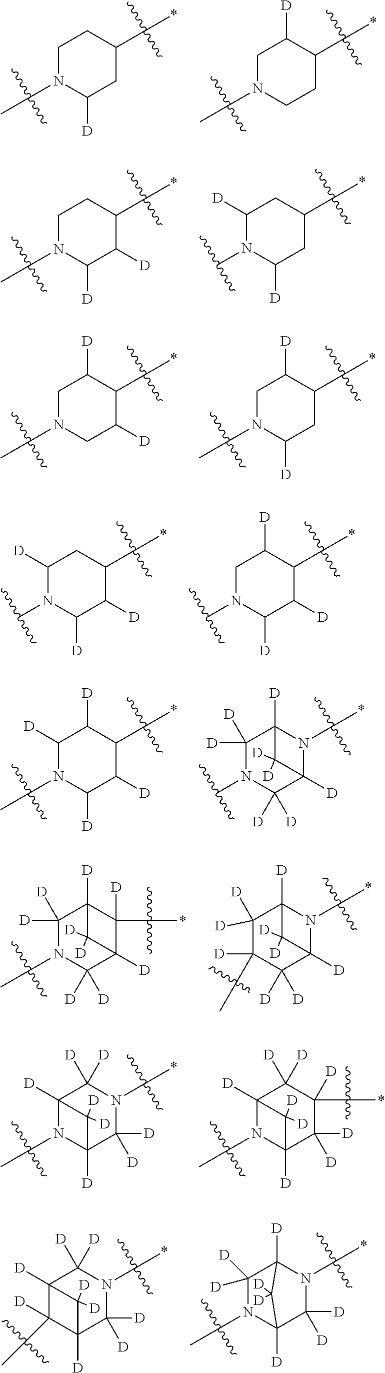 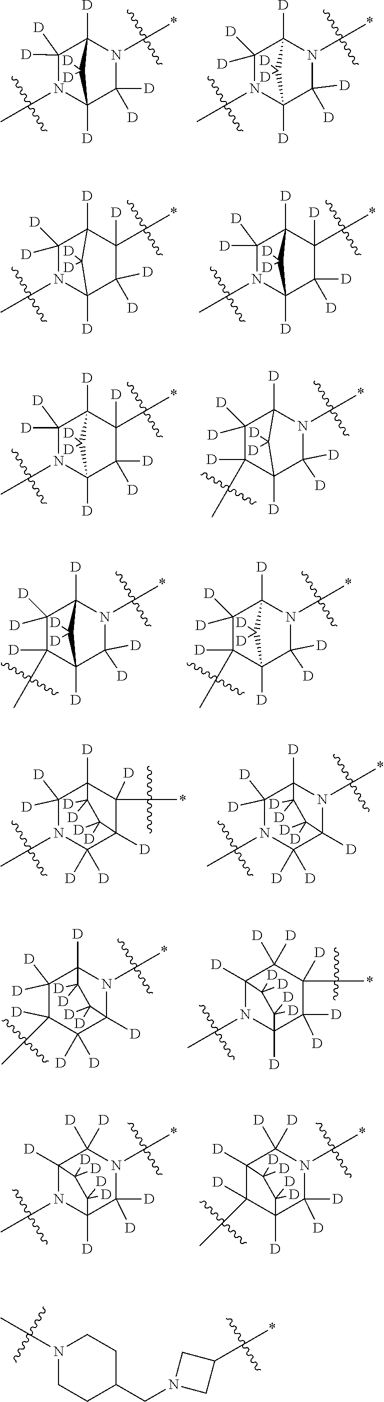 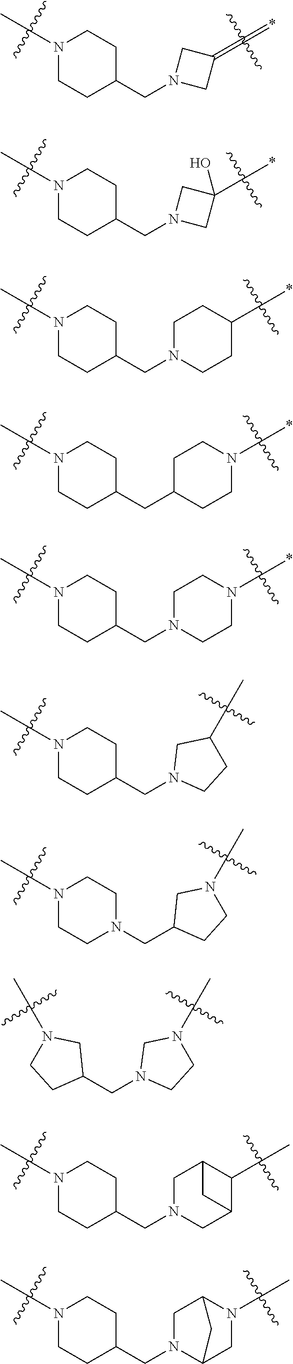 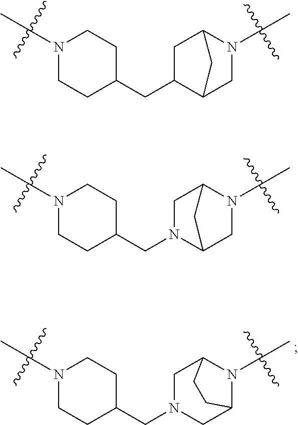 [SMILES: *N1CCC(CC2CC3CC2CN3*)CC1 ; *N1CCC(CN2CC3CC2CN3*)CC1 ; *N1CCC(CN2CC3CCC(C2)N3*)CC1]
wherein the groups are each independently optionally substituted with one or more substituents selected from the group consisting of deuterium, C₁₋₆ alkyl, deuterated C₁₋₆ alkyl, halogenated C₁₋₆ alkyl, C₁₋₆ alkoxy, halogenated C₁₋₆ alkoxy, halogen, amino, hydroxy, mercapto, cyano, C₂₋₆ alkynyl, C₂₋₆ alkenyl, optionally deuterated C₃₋₆ cycloalkyl, R_{c12}—R_{c11}—, and any combination thereof, wherein R^{c11} is optionally substituted C₁₋₆ alkylene or optionally substituted C₂₋₆ alkenylene, and R_{c12} represents halogen, R_{c13}R_{c14}N—, hydroxy, carboxyl, ethynyl, or vinyl, wherein R_{c13} and R_{c14} are the same or different and independently represent hydrogen or C₁₋₃ alkyl, and the C₁₋₆ alkylene and C₂₋₆ alkenylene are each independently optionally substituted with one or more substituents selected from the group consisting of deuterium, halogen, hydroxy, mercapto, cyano, amino, nitro, C₁₋₆ alkyl, halogenated C₁₋₆ alkyl, deuterated C₁₋₆ alkyl, C₁₋₆ alkoxy, or halogenated C₁₋₆ alkoxy; and symbol * indicates the point of attachment to LIN.

9. The compound of Formula (I) or salts, enantiomers, stereoisomers, solvates, isotopically enriched analogs, prodrugs, or polymorphs thereof as claimed in claim 1, wherein the ULM represents a structure of the following Formula (ULM-1-1), Formula (ULM-1-2), Formula (ULM-1-3), or Formula (ULM-1-4): 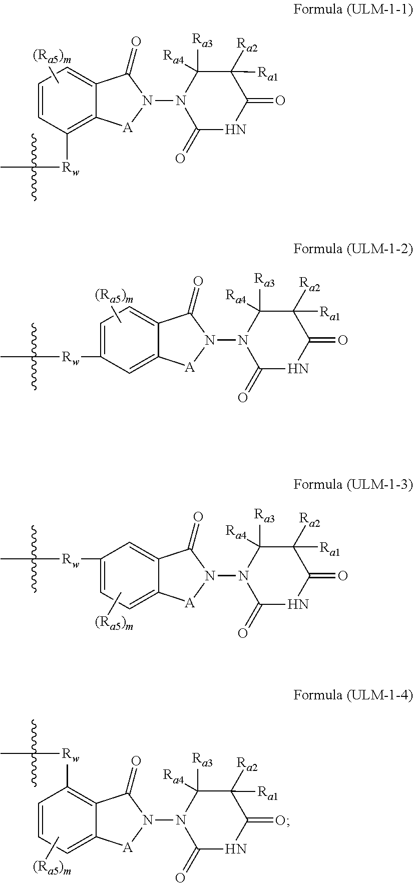
wherein
A represents CO, CH₂, or CD₂;
Rₐ₁, Rₐ₂, Rₐ₃, and Rₐ₄ are the same or different and independently represent hydrogen, deuterium, halogen, C₁₋₆ alkyl, halogenated C₁₋₆ alkyl, deuterated C₁₋₆ alkyl, C₁₋₆ alkoxy, deuterated C₁₋₆ alkoxy, or halogenated C₁₋₆ alkoxy;
(Rₐ₅)ₘ indicates that the isoindoline ring to which it is attached is optionally substituted with m Rₐ₅, with each Rₐ₅ being the same or different and each independently representing halogen, hydroxy, mercapto, nitro, amino, cyano, C₁₋₆ alkyl, halogenated C₁₋₆ alkyl, deuterated C₁₋₆ alkyl, C₁₋₆ alkoxy, deuterated C₁₋₆ alkoxy, halogenated C₁₋₆ alkoxy, C₂₋₆ alkenyl or C₂₋₆ alkynyl;
m represents an integer of 0, 1, 2, 3, or 4;
R_{w} represents O, S, S(O), S(O)₂, alkenylene, alkynylene, or N(Rₐ₆), wherein Rₐ₆ represents H or C₁₋₃ alkyl, or R_{w} represents a bond, or
R_{w} represents: 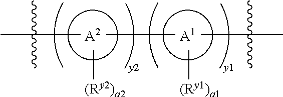
wherein each ring A¹ is the same or different and independently represents nitrogen-containing heterocyclylene, arylene, or heteroarylene, y1 represents an integer of 1 or 2, and (R^{y1})ₐ₁ indicates that each ring A¹ is independently optionally substituted with a1 R^{y1} substituents, wherein each R^{y1} independently represents deuterium, C₁₋₆ alkyl, deuterated C₁₋₆ alkyl, halogenated C₁₋₆ alkyl, C₁₋₆ alkoxy, halogen, amino, hydroxy, mercapto, cyano, carboxyl, C₂₋₆ alkynyl, or C₂₋₆ alkenyl, and a1 represents an integer of 0-20;
each ring A² is the same or different and independently represents heterocyclylene, cycloalkylene, arylene, or heteroarylene, y2 represents an integer of 0 or 1, and (R^{y2})ₐ₂ indicates that each ring A² is independently optionally substituted with a2 R^{y2} substituents, wherein each R^{y2} independently represents deuterium, C₁₋₆ alkyl, deuterated C₁₋₆ alkyl, halogenated C₁₋₆ alkyl, C₁₋₆ alkoxy, halogen, amino, hydroxy, mercapto, cyano, carboxyl, C₂₋₆ alkynyl, or C₂₋₆ alkenyl, and a2 represents an integer of 0-20.

10. The compound of Formula (I) or salts, enantiomers, stereoisomers, solvates, isotopically enriched analogs, prodrugs, or polymorphs thereof as claimed in claim 1, wherein the ULM represents a structure of the following Formula (ULM-2-1), Formula (ULM-2-2), Formula (ULM-2-3), or Formula (ULM-2-4): 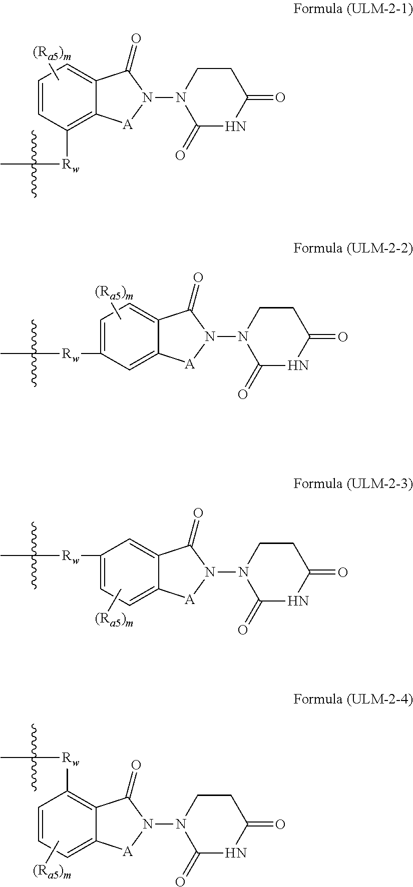
wherein A, (Rₐ₅)ₘ, and R_{w} are as defined in claim 1.

11. The compound of Formula (I) or salts, enantiomers, stereoisomers, solvates, isotopically enriched analogs, prodrugs, or polymorphs thereof as claimed in claim 1, wherein
(i) each ring A¹ independently represents 4-to 20-membered nitrogen-containing heterocyclylene, C₅₋₂₀ arylene, or 5-to 20-membered heteroarylene; preferably each ring A¹ independently represents:
piperidinylene, piperazinylene, morpholinylene, azetidinylene, pyrrolidinylene, imidazolidylene, pyrazolidylene, oxazolidinylene, thiazolidinylene, thiomorpholinylene, azepanylene, diazacycloheptanylene, azacyclooctylene, diazacyclooctylene, azabicyclo[3.1.1]heptanylene, azabicyclo[2.2.1]heptanylene, azabicyclo[3.2.1]octanylene, azabicyclo[2.2.2]octanylene, diazabicyclo[3.1.1]heptanylene, diazabicyclo[2.2.1]heptanylene, diazabicyclo[3.2.1]octanylene, diazabicyclo[2.2.2]octanylene, 3-azaspiro[5.5]undecanylene, 8-azaspiro[4.5]decanylene, 2-oxa-8-azaspiro[4.5]decanylene, 3-methyl-2-oxa-8-azaspiro[4.5]decanylene, phenylene, naphthylene, furanylene, oxazolylene, isoxazolylene, oxadiazolylene, thienylene, thiazolylene, isothiazolylene, thiadiazolylene, pyrrolylene, imidazolylene, pyrazolylene, triazolylene, pyridylene, pyrimidinylene, pyridazinylene, pyrazinylene, indolylene, isoindolylene, benzofuranylene, isobenzofuranylene, benzothienylene, indazolylene, benzimidazolylene, benzoxazolylene, benzisoxazolylene, benzothiazolylene, benzisothiazolylene, benzotriazolylene, benzo[2,1,3]oxadiazolylene, benzo[2,1,3]thiadiazolylene, benzo[1,2,3]thiadiazolylene, quinolinylene, isoquinolinylene, naphthyridinylene, cinnolinylene, quinazolinylene, quinoxalinylene, phthalazinylene, pyrazolo[1,5-a]pyridylene, pyrazolo[1,5-alpyrimidinylene, imidazo[1,2-a]pyridylene, 1H-pyrrolo[3,2-b]pyridylene, 1H-pyrrolo[2,3-b]pyridylene, pyrrolo[2,1-b]thiazolylene, imidazo[2,1-b]thiazolylene, or 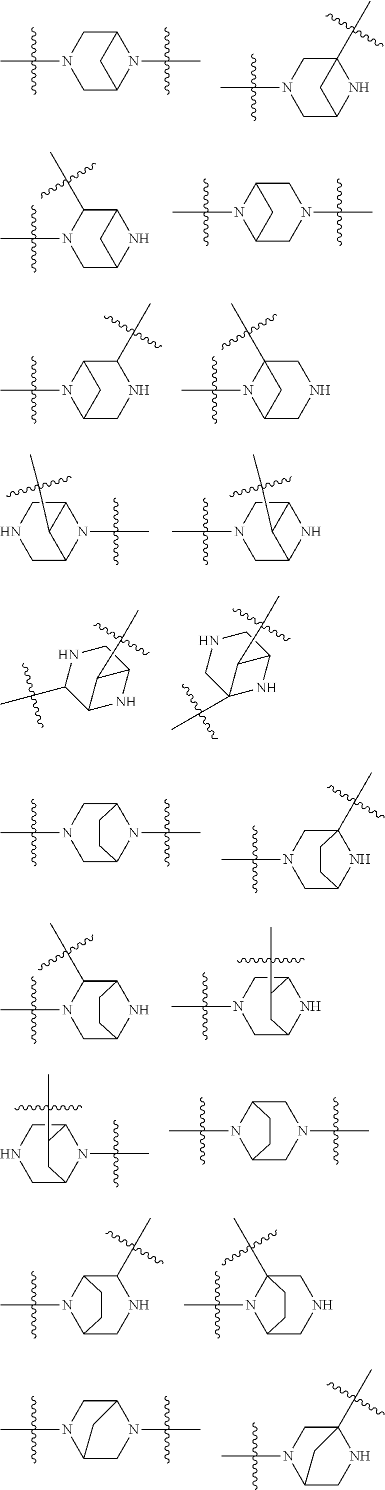 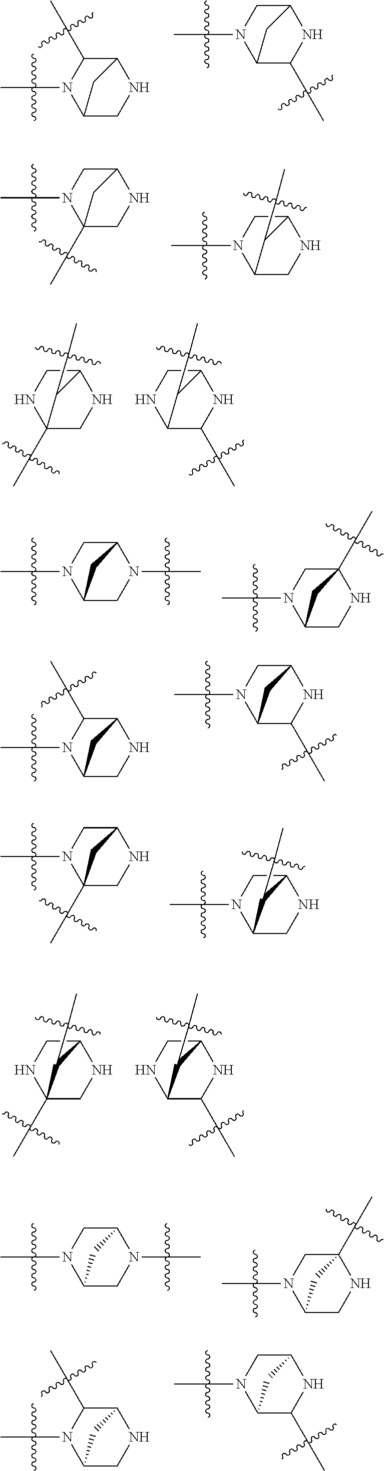 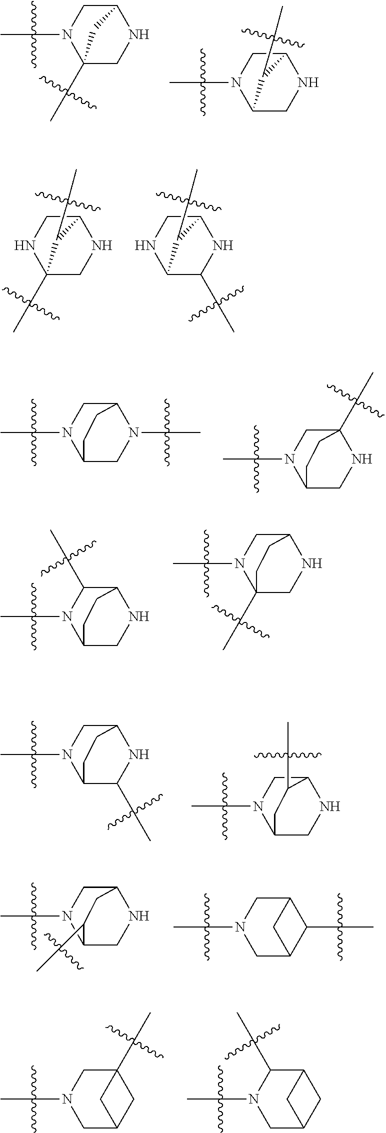 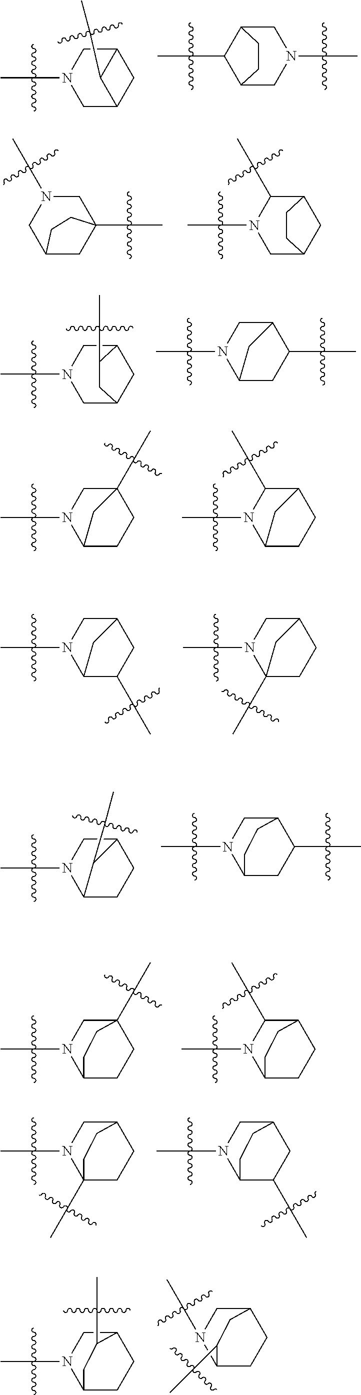 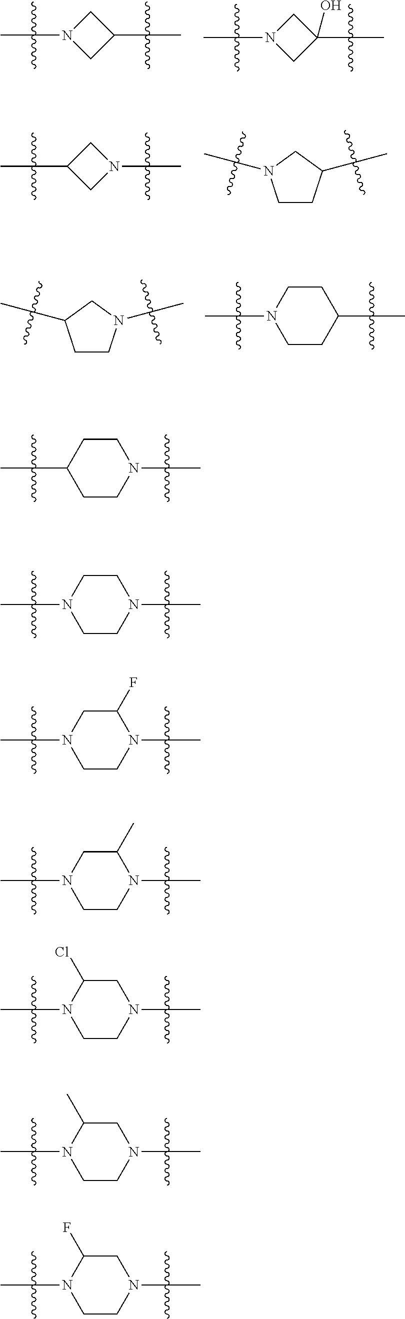 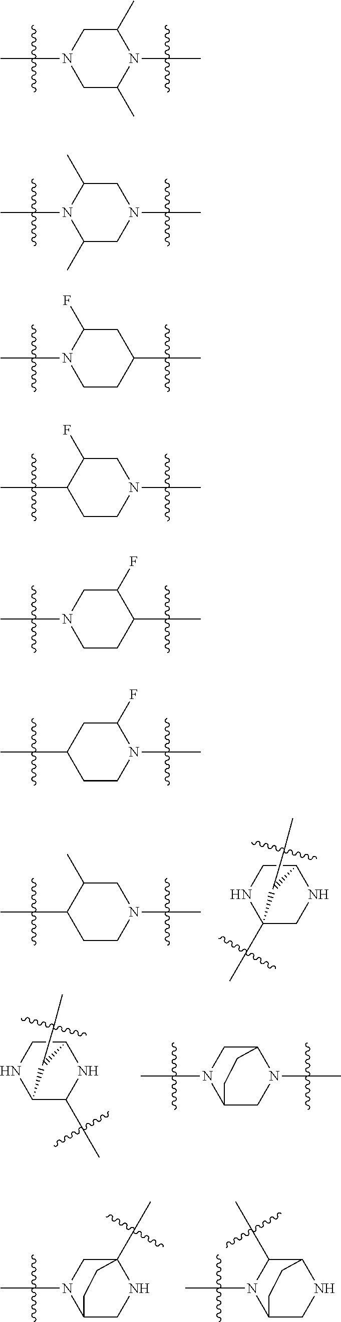 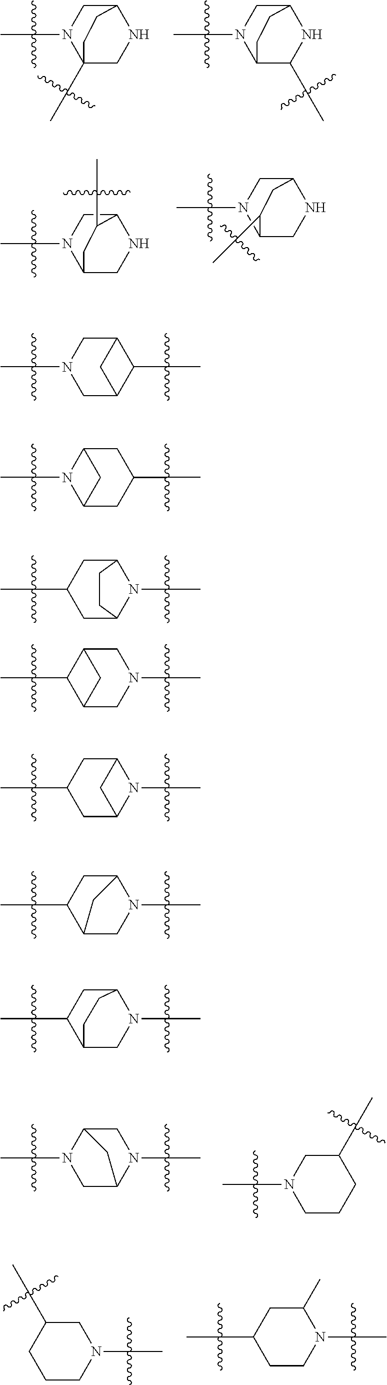 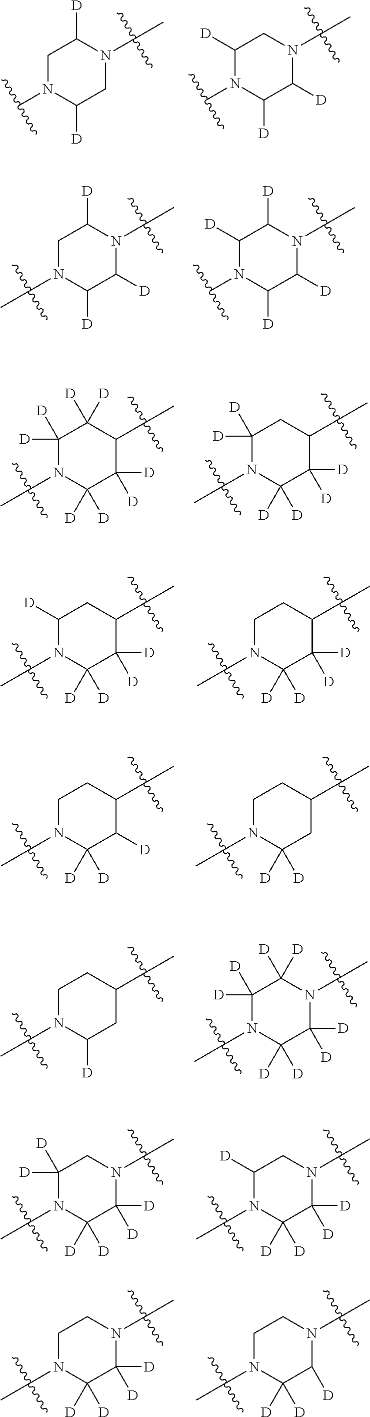 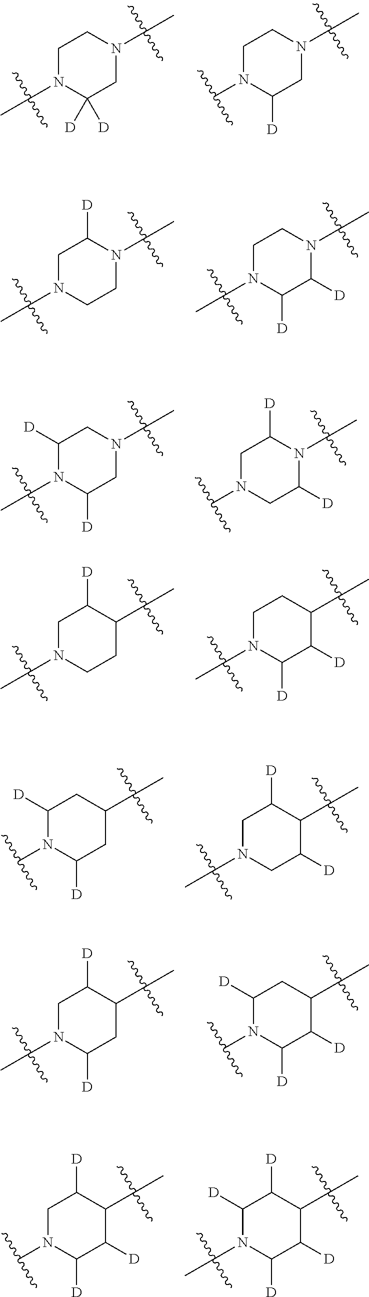 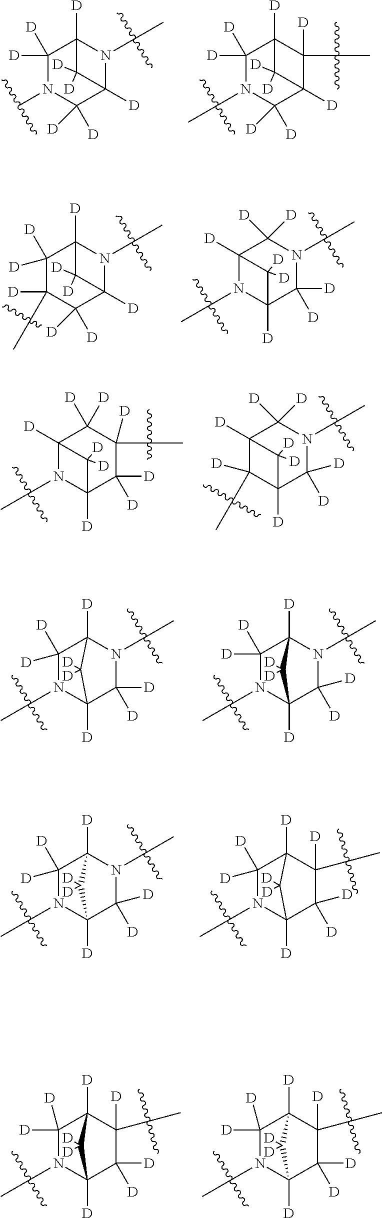 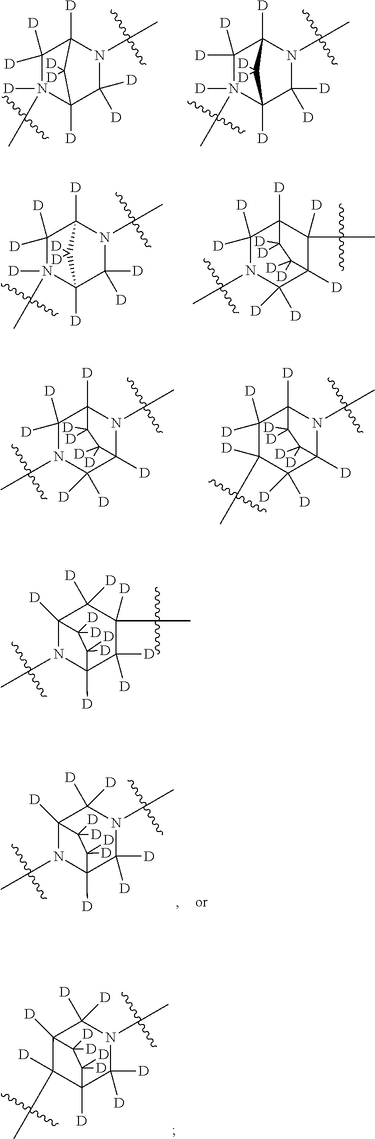
each ring A¹ is independently optionally substituted with a1 R^{y1} substituents, and each R^{y1} independently represents deuterium, C₁₋₆ alkyl, deuterated C₁₋₆ alkyl, halogenated C₁₋₆ alkyl, C₁₋₆ alkoxy, halogen, amino, hydroxy, mercapto, cyano, carboxyl, C₂₋₆ alkynyl, or C₂₋₆ alkenyl;
a1 represents an integer of 0-20, and/or
(ii) each ring A² independently represents 4-to 20-membered heterocyclylene, C₃₋₂₀ cycloalkylene, C₅₋₂₀ arylene, or 5-to 20-membered heteroarylene; preferably each ring A² independently represents:
azetidinylene, oxetanylene, pyrrolidinylene, imidazolidinylene, pyrazolidylene, tetrahydrofuranylene, tetrahydropyranylene, tetrahydrothienylene, tetrahydrothiopyranylene, oxazolidinylene, thiazolidinylene, piperidinylene, piperazinylene, morpholinylene, thiomorpholinylene, azacycloheptanylene, azacyclooctylene, dioxacyclohexylene, azacycloheptanylene, azacyclooctylene, diazacycloheptanylene, diazacyclooctylene, 6-azabicyclo[3.1.1]heptanylene, 2,5-diazabicyclo[2.2.1]heptanylene, 3,6-diazabicyclo[3.1.1]heptanylene, 3-azabicyclo[3.2.1]octanylene, 3,8-diazabicyclo[3.2.1]octanylene, 3,8-diazabicyclo[3.2.1]octanylene, 2,5-diazabicyclo[2.2.2]octanylene, 5-to 20-membered azaspirocycloalkylene, cyclopropylene, cyclobutylene, cyclopentylene, cyclopentenylene, cyclohexylene, cyclohexenylene, cycloheptylene, cyclooctylene, decalinylene, octahydropentalenylene, octahydro-1H-indenylene, 2,3-dihydro-1H-indenylene, C₅-15 spiro-cycloalkylene, adamantanylene, noradamantanylene, norbornylene, phenylene, naphthylene, furanylene, oxazolylene, isoxazolylene, oxadiazolylene, thienylene, thiazolylene, isothiazolylene, thiadiazolylene, pyrrolylene, imidazolylene, pyrazolylene, triazolylene, pyridylene, pyrimidinylene, pyridazinylene, pyrazinylene, indolylene, isoindolylene, benzofuranylene, isobenzofuranylene, benzothienylene, indazolylene, benzimidazolylene, benzoxazolylene, benzisoxazolylene, benzothiazolylene, benzisothiazolylene, benzotriazolylene, benzo[2,1,3]oxadiazolylene, benzo[2,1,3]thiadiazolylene, benzo[1,2,3]thiadiazolylene, quinolinylene, isoquinolinylene, naphthyridinylene, cinnolinylene, quinazolinylene, quinoxalinylene, phthalazinylene, pyrazolo[1,5-a]pyridylene, pyrazolo[1,5-a]pyrimidinylene, imidazo[1,2-a]pyridylene, 1H-pyrrolo[3,2-b]pyridylene, 1H-pyrrolo[2,3-b]pyridylene, pyrrolo[2,1-b]thiazolylene, imidazo[2,1-b]thiazolylene, or 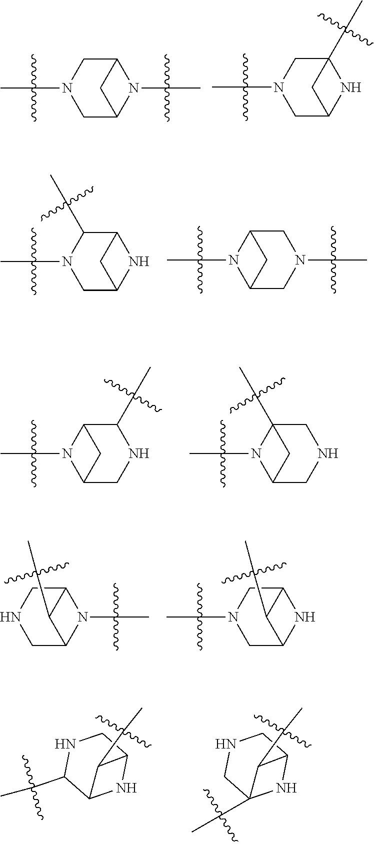 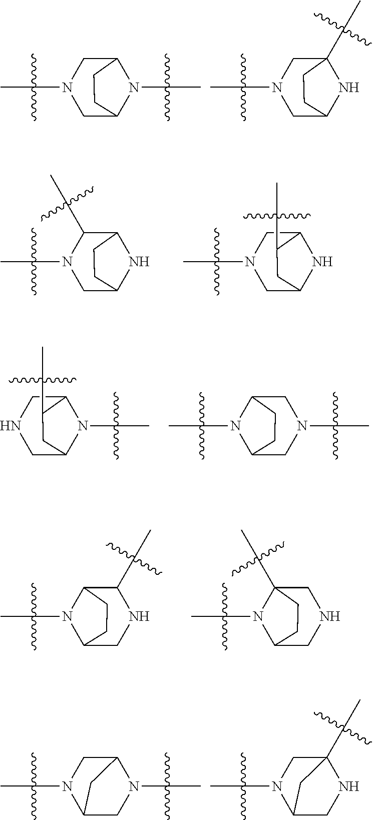 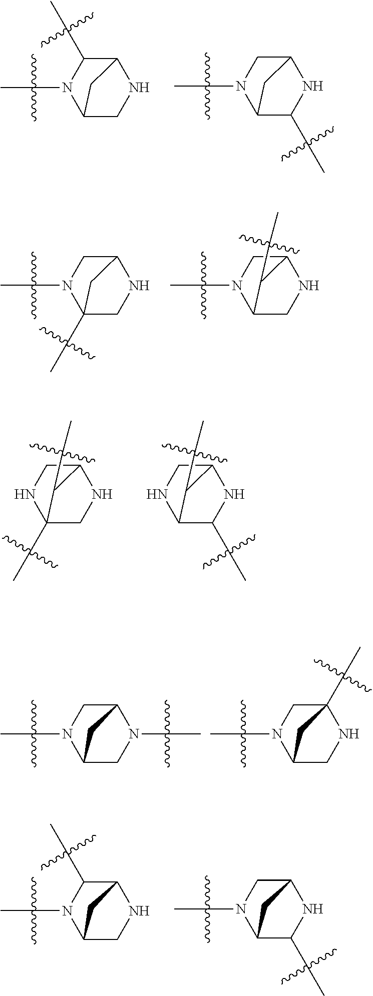 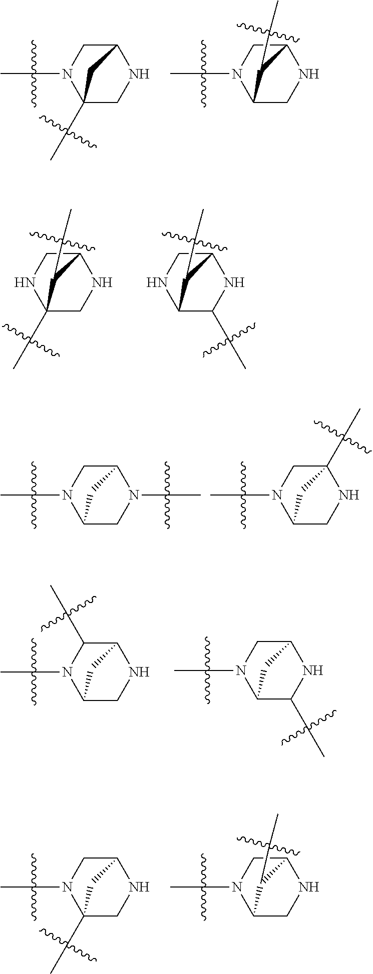 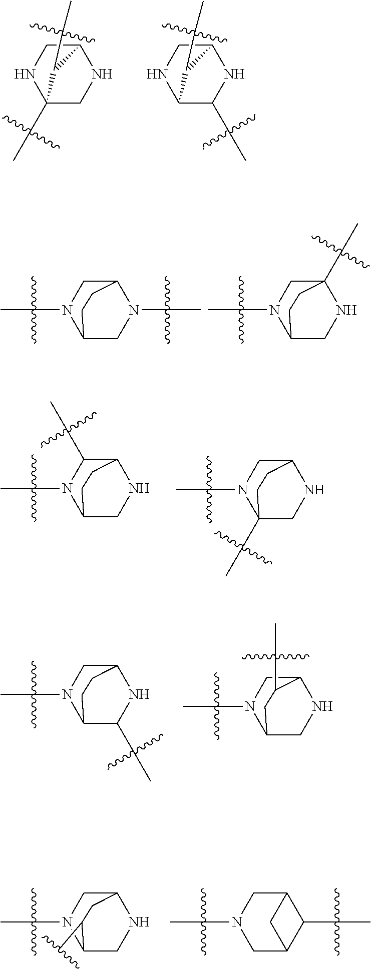 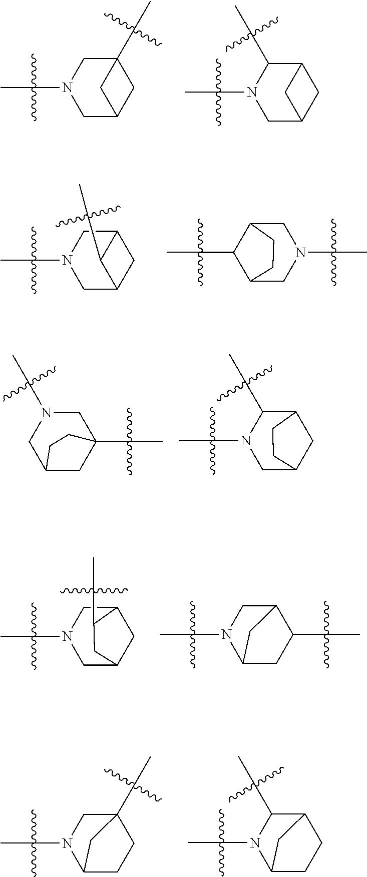 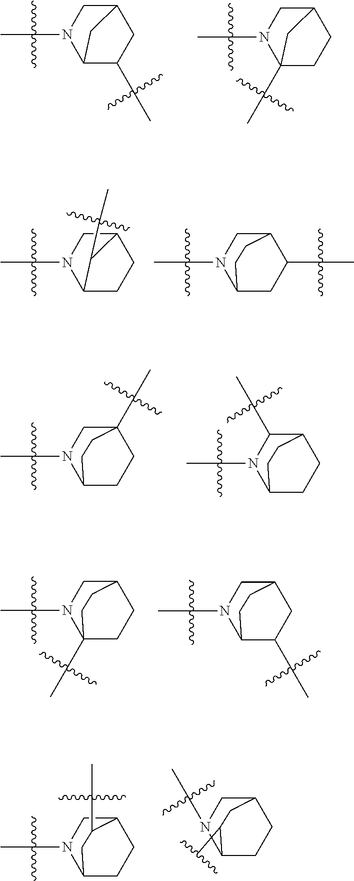 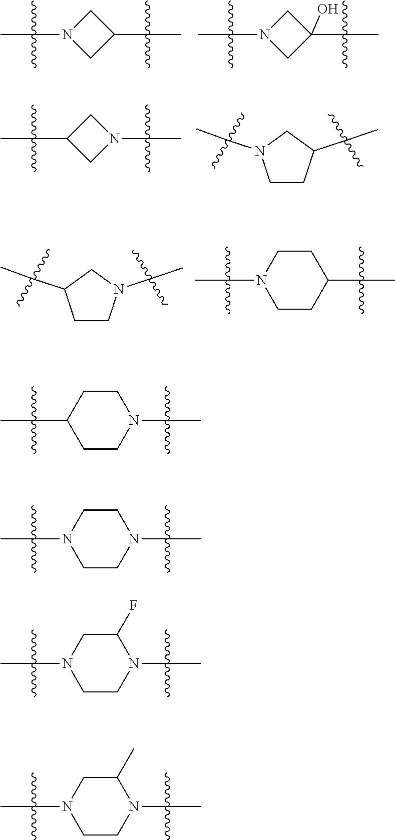 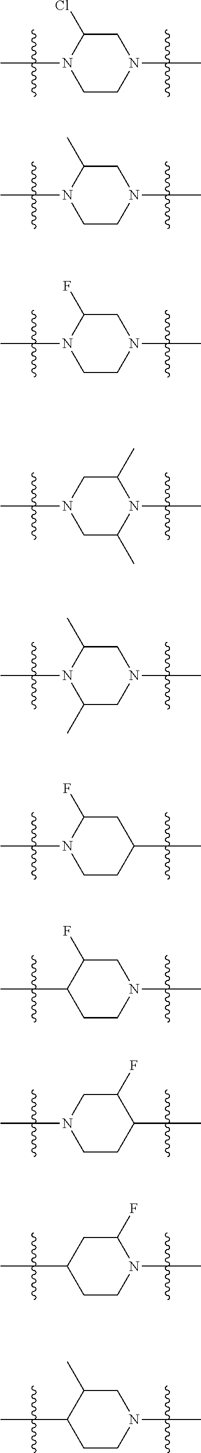 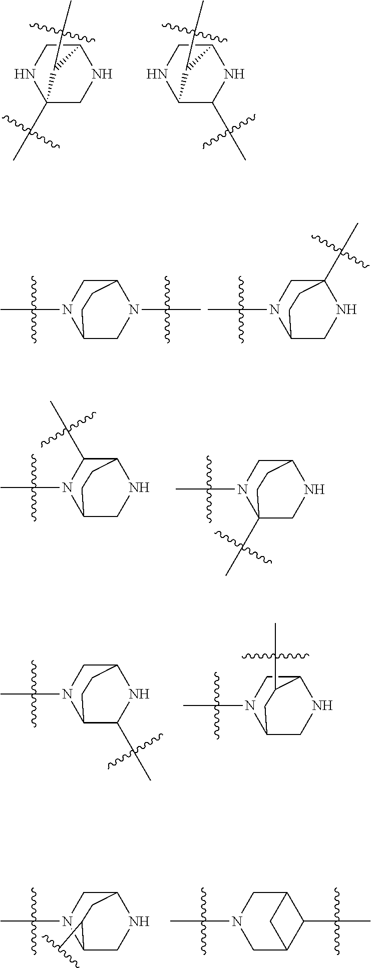 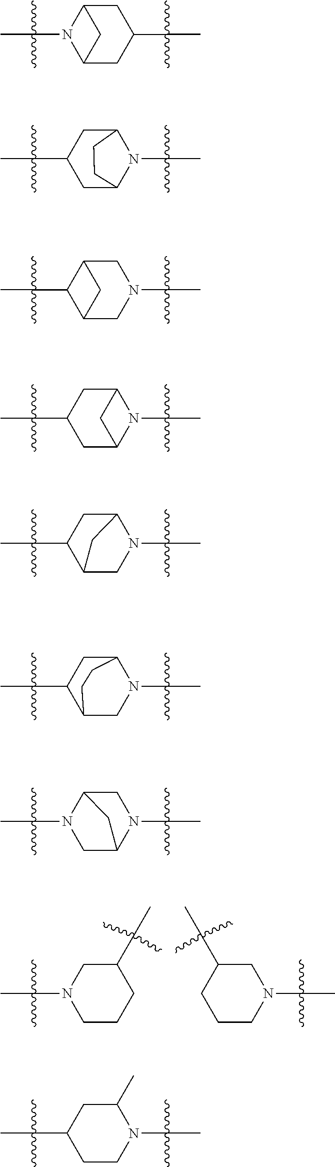 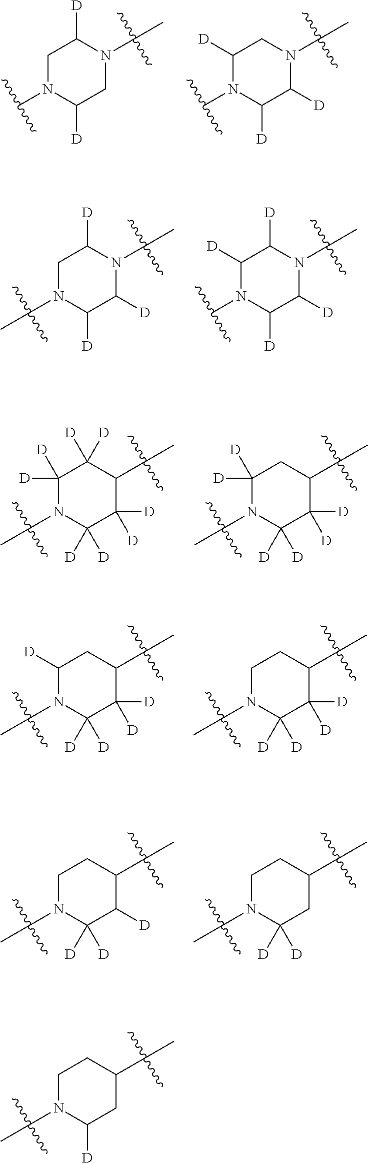 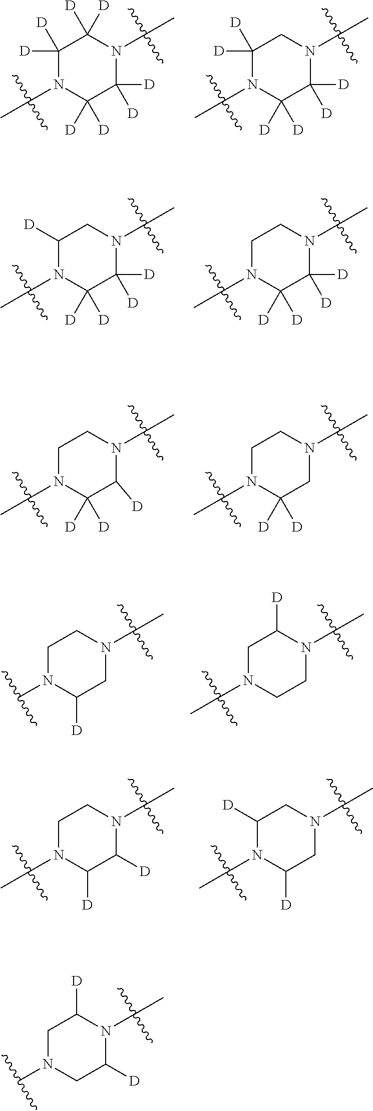 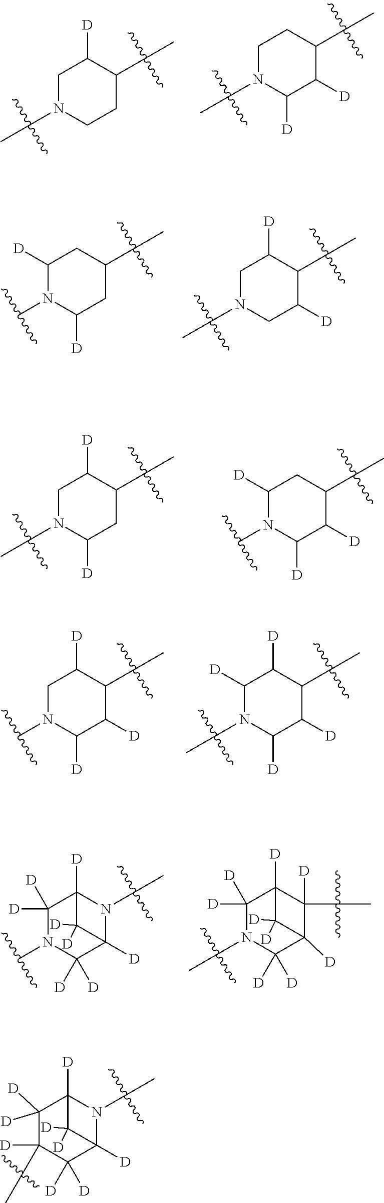 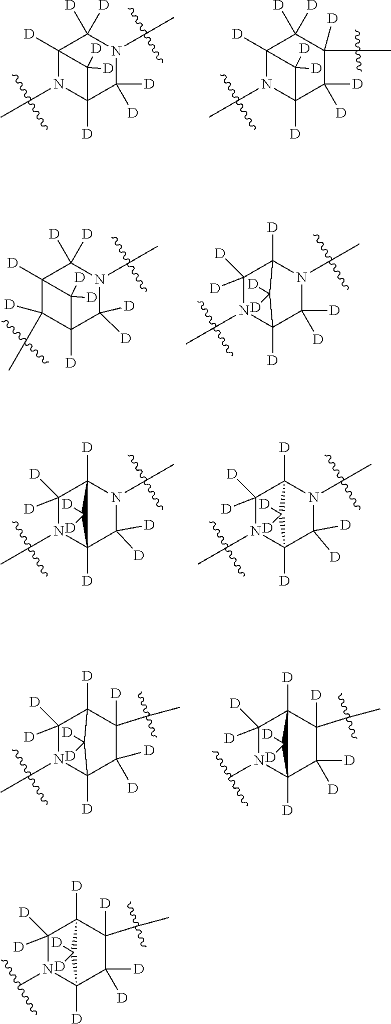 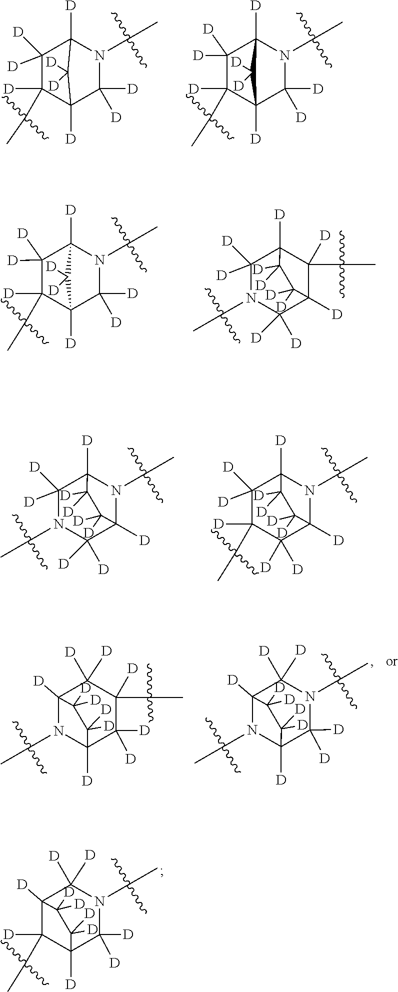
each ring A² is independently optionally substituted with a2 R^{y2} groups, wherein each R^{y2} is independently deuterium, C₁₋₆ alkyl, deuterated C₁₋₆ alkyl, halogenated C₁₋₆ alkyl, C₁₋₆ alkoxy, halogen, amino, hydroxy, mercapto, cyano, carboxyl, C₂₋₆ alkynyl, or C₂₋₆ alkenyl, and
a2 represents an integer of 0-20.

12. The compound of Formula (I) or salts, enantiomers, stereoisomers, solvates, isotopically enriched analogs, prodrugs, or polymorphs thereof as claimed in claim 1, wherein R_{w} represents: 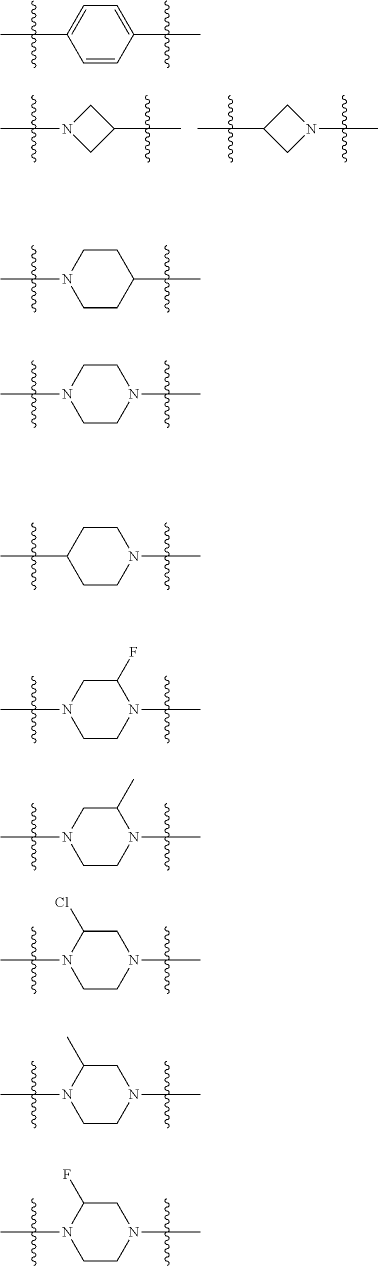 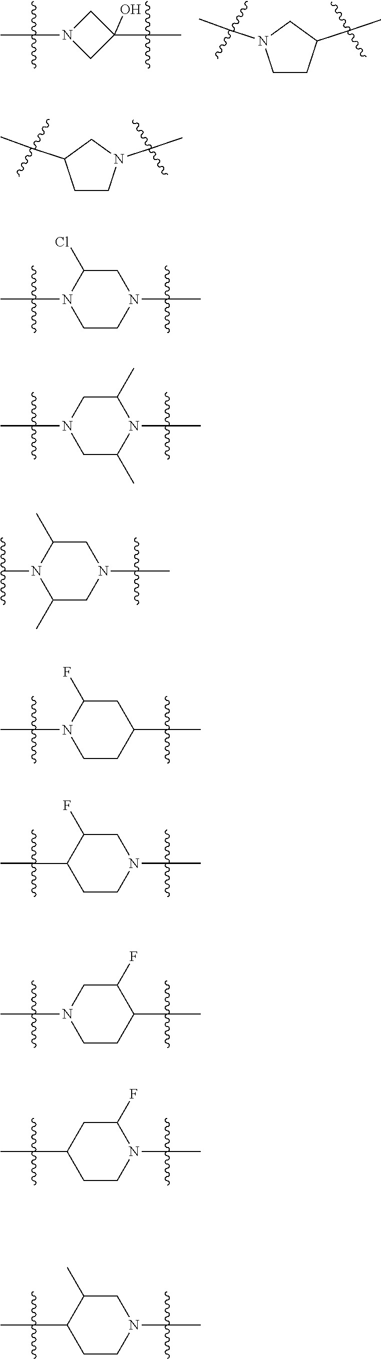 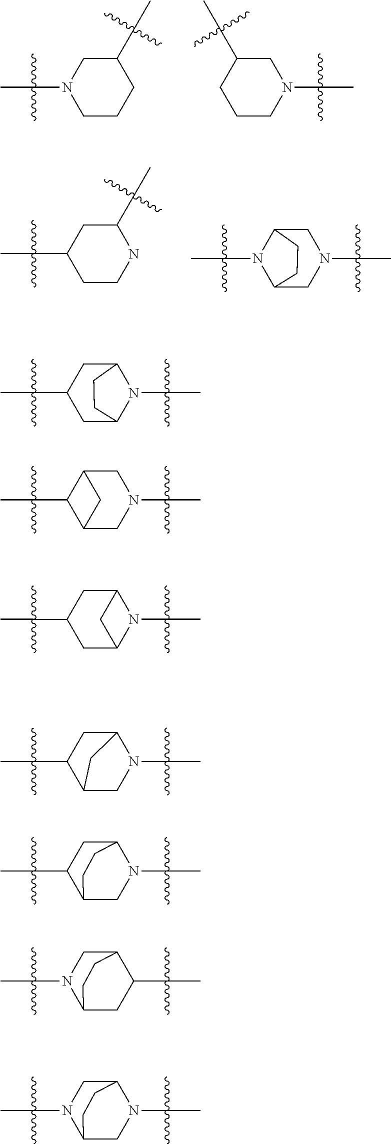 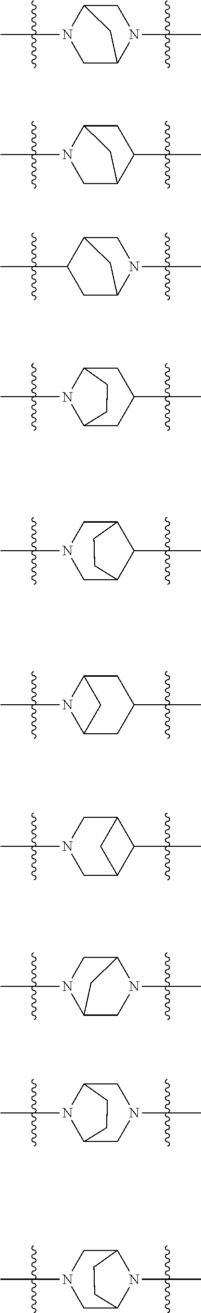 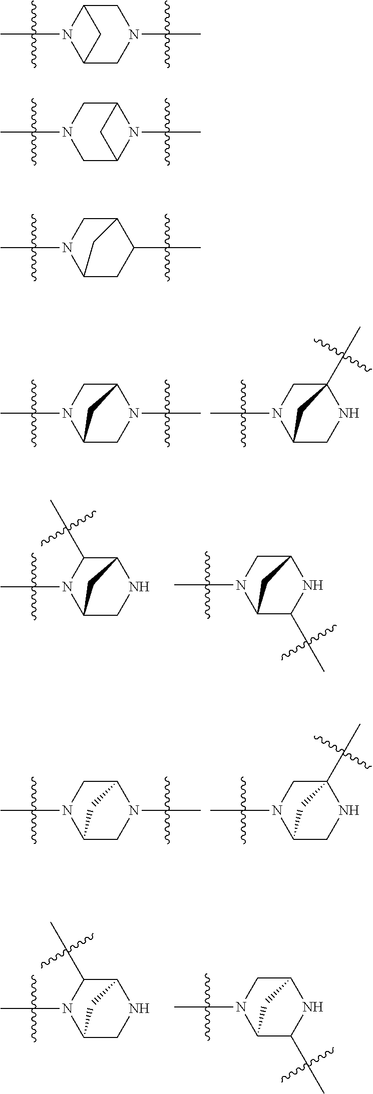 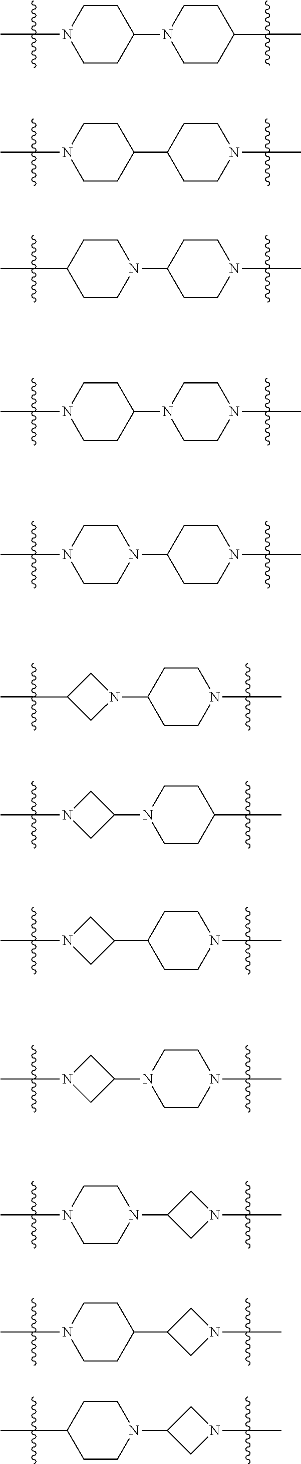 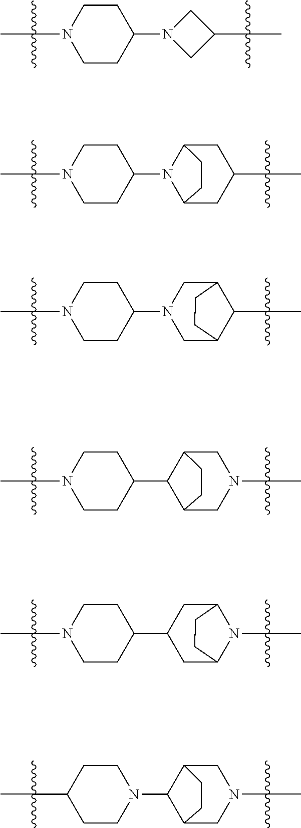 [SMILES: *C1CN(C2CCN(*)CC2)C1 ; *C1CC2CCC(C1)N2C1CCN(*)CC1 ; *C1C2CCC1CN(C1CCN(*)CC1)C2 ; *N1CCC(C2C3CCC2CN(*)C3)CC1 ; *N1CCC(C2CC3CCC(C2)N3*)CC1 ; *C1CCN(C2C3CCC2CN(*)C3)CC1] 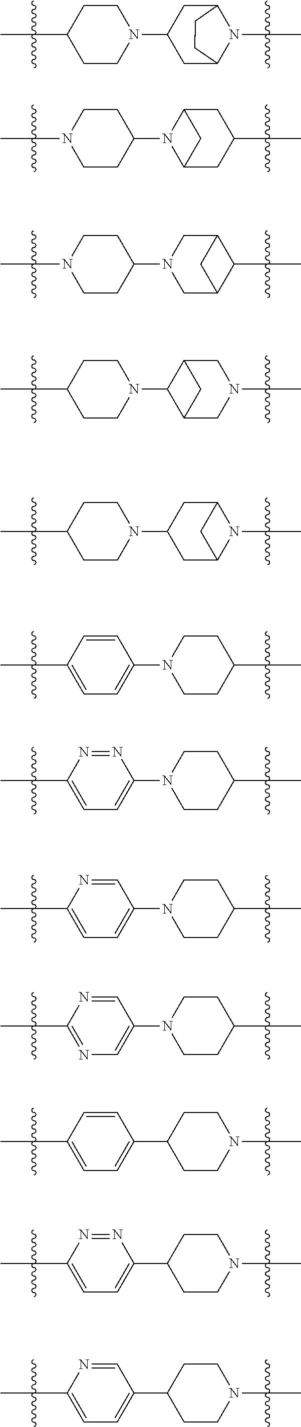 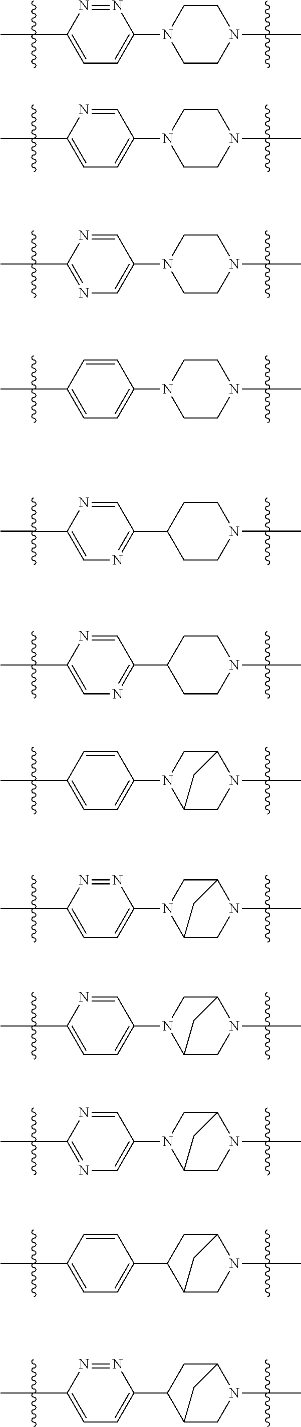 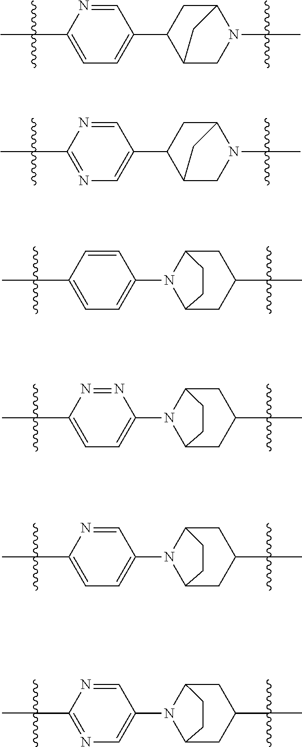 [SMILES: *c1ccc(C2CC3CC2CN3*)cn1 ; *c1ncc(C2CC3CC2CN3*)cn1 ; *c1ccc(N2C3CCC2CC(*)C3)cc1 ; *c1ccc(N2C3CCC2CC(*)C3)nn1 ; *c1ccc(N2C3CCC2CC(*)C3)cn1 ; *c1ncc(N2C3CCC2CC(*)C3)cn1] 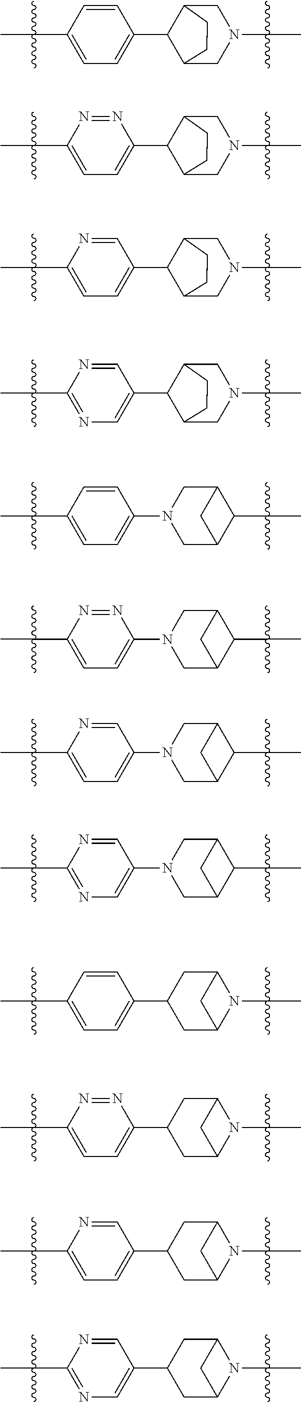 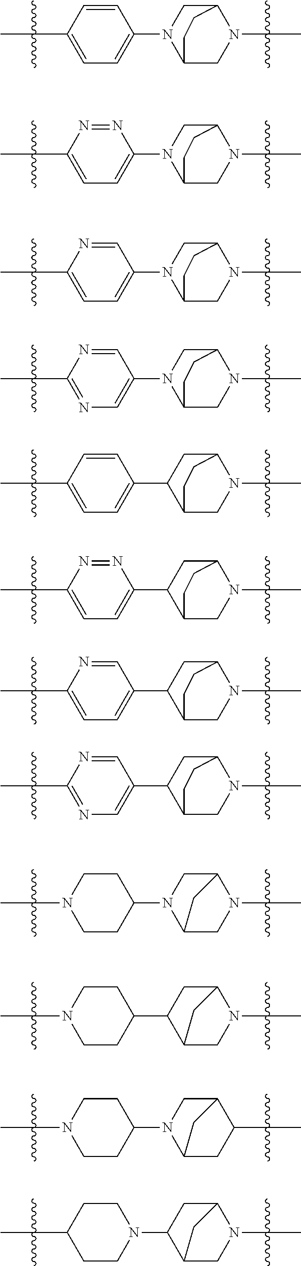 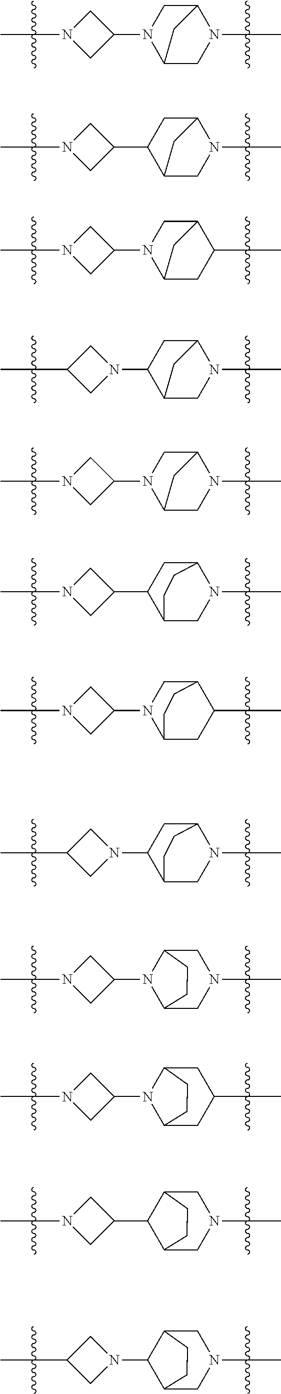 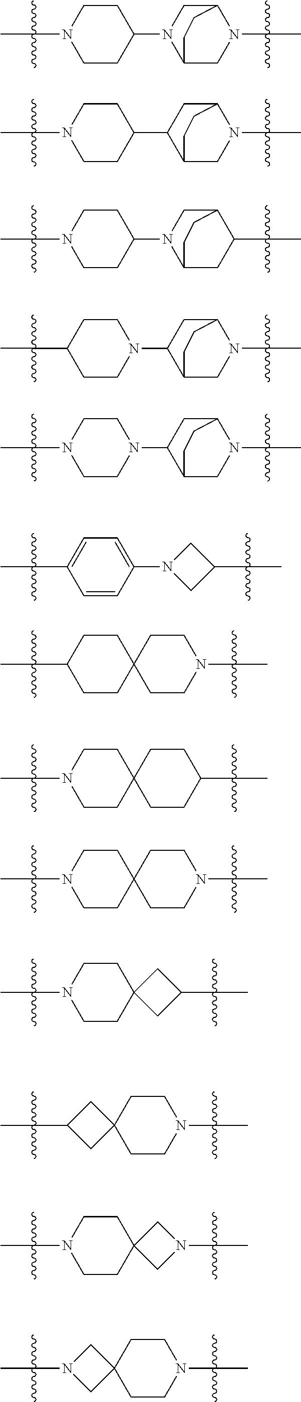 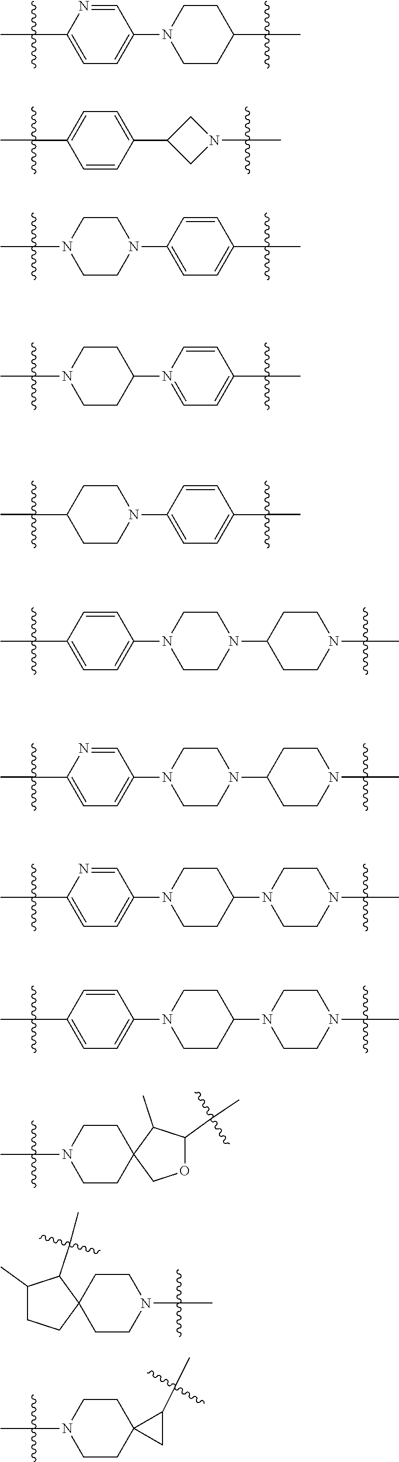 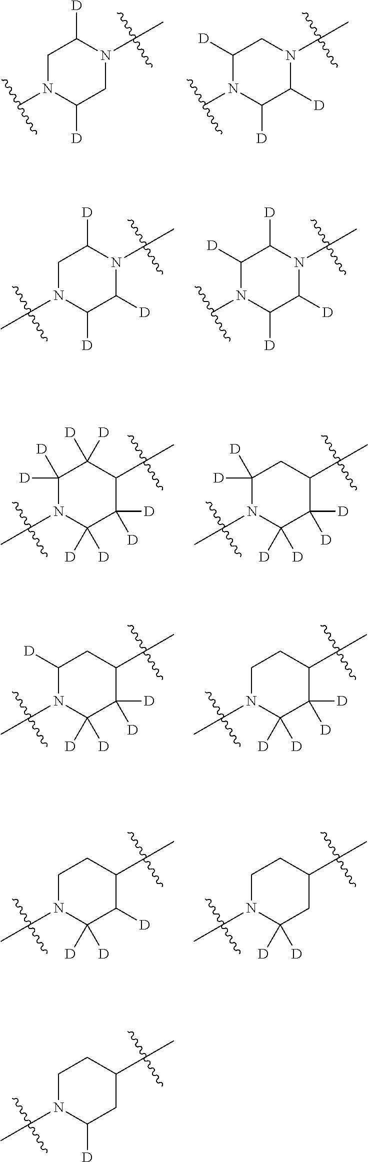 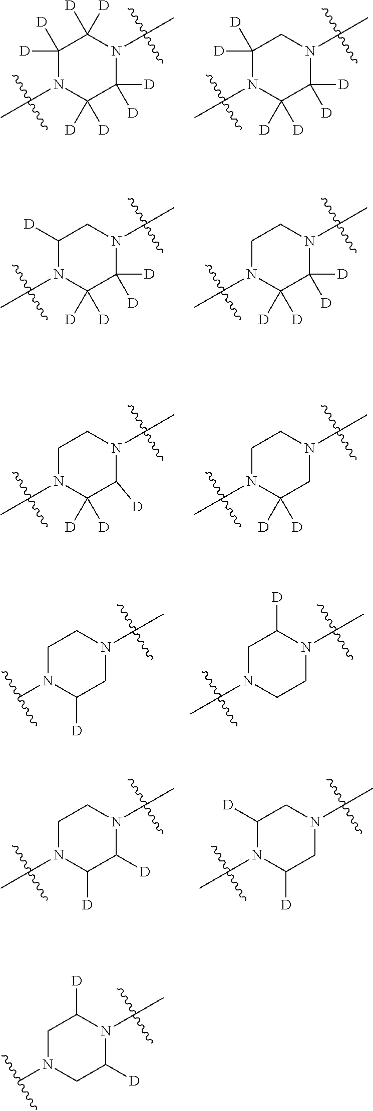 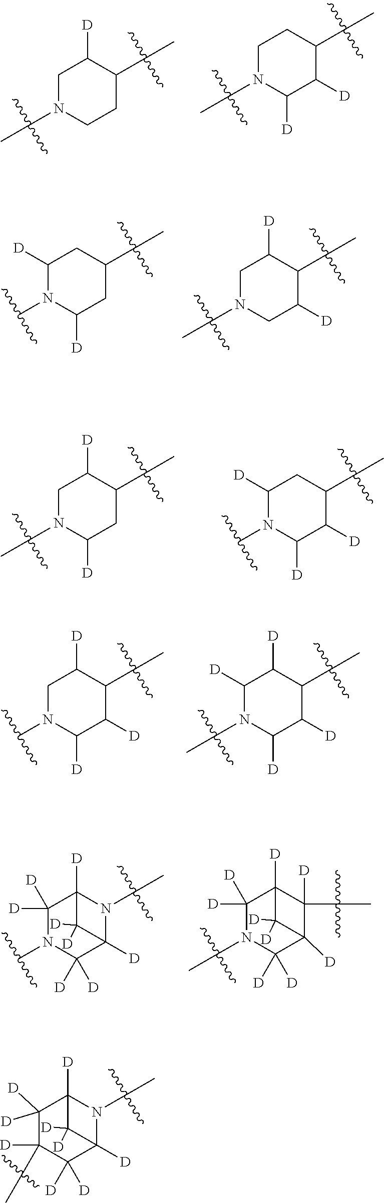 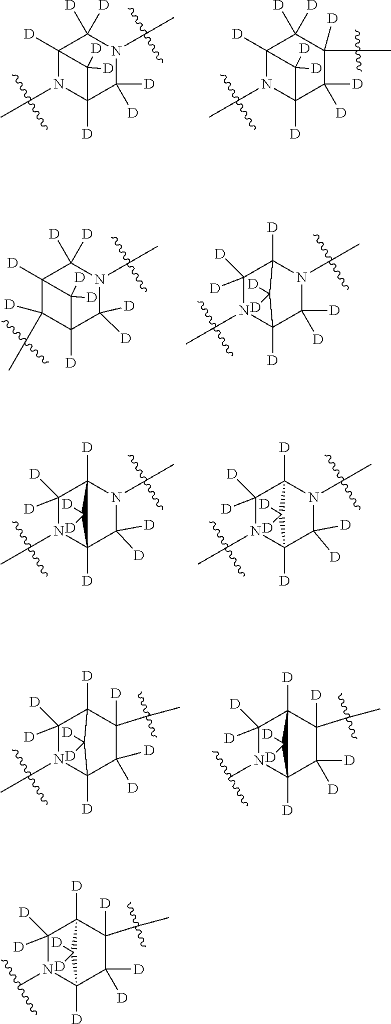 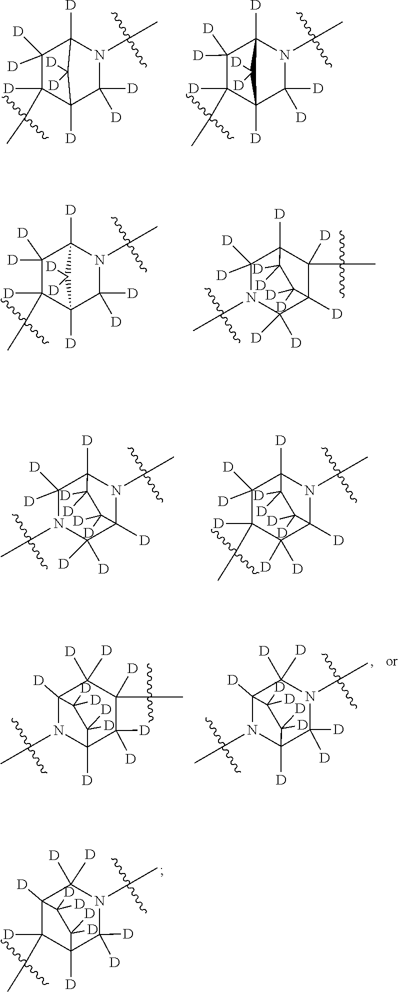
wherein the groups are optionally substituted with a substituent selected from the group consisting of deuterium, C₁₋₆ alkyl, deuterated C₁₋₆ alkyl, halogenated C₁₋₆ alkyl, C₁₋₆ alkoxy, halogen, amino, hydroxy, mercapto, cyano, carboxyl, C₂₋₆ alkynyl, or C₂₋₆ alkenyl.

13. The compound of Formula (I) or salts, enantiomers, stereoisomers, solvates, isotopically enriched analogs, prodrugs, or polymorphs thereof as claimed in claim 1, wherein
each ring A³ is the same or different and independently represents 4-to 20-membered heterocyclylene, C₃₋₂₀cycloalkylene, C₅₋₂₀ arylene, or 5-to 20-membered heteroarylene, preferably each ring A³ independently represents:
azetidinylene, oxetanylene, pyrrolidinylene, imidazolidinylene, pyrazolidylene, tetrahydrofuranylene, tetrahydropyranylene, tetrahydrothienylene, tetrahydrothiopyranylene, oxazolidinylene, thiazolidinylene, piperidinylene, piperazinylene, morpholinylene, thiomorpholinylene, azacycloheptanylene, azacyclooctylene, dioxacyclohexylene, azacycloheptanylene, azacyclooctylene, diazacycloheptanylene, diazacyclooctylene, 6-azabicyclo[3.1.1]heptanylene, 2,5-diazabicyclo[2.2.1]heptanylene, 3,6-diazabicyclo[3.1.1]heptanylene, 3-azabicyclo[3.2.1]octanylene, 3,8-diazabicyclo[3.2.1]octanylene, 3,8-diazabicyclo[3.2.1]octanylene, 2,5-diazabicyclo[2.2.2]octanylene, 5-to 20-membered azaspirocycloalkylene, cyclopropylene, cyclobutylene, cyclopentylene, cyclopentenylene, cyclohexylene, cyclohexenylene, cycloheptylene, cyclooctylene, decalinylene, octahydropentalenylene, octahydro-1H-indenylene, 2,3-dihydro-1H-indenylene, C₅-15 spiro-cycloalkylene, adamantanylene, noradamantanylene, norbornylene, phenylene, naphthylene, furanylene, oxazolylene, isoxazolylene, oxadiazolylene, thienylene, thiazolylene, isothiazolylene, thiadiazolylene, pyrrolylene, imidazolylene, pyrazolylene, triazolylene, pyridylene, pyrimidinylene, pyridazinylene, pyrazinylene, indolylene, isoindolylene, benzofuranylene, isobenzofuranylene, benzothienylene, indazolylene, benzimidazolylene, benzoxazolylene, benzisoxazolylene, benzothiazolylene, benzisothiazolylene, benzotriazolylene, benzo[2,1,3]oxadiazolylene, benzo[2,1,3]thiadiazolylene, benzo[1,2,3]thiadiazolylene, quinolinylene, isoquinolinylene, naphthyridinylene, cinnolinylene, quinazolinylene, quinoxalinylene, phthalazinylene, pyrazolo[1,5-a]pyridylene, pyrazolo[1,5-a]pyrimidinylene, imidazo[1,2-a]pyridylene, 1H-pyrrolo[3,2-b]pyridylene, 1H-pyrrolo[2,3-b]pyridylene, pyrrolo[2,1-b]thiazolylene, imidazo[2,1-b]thiazolylene, or 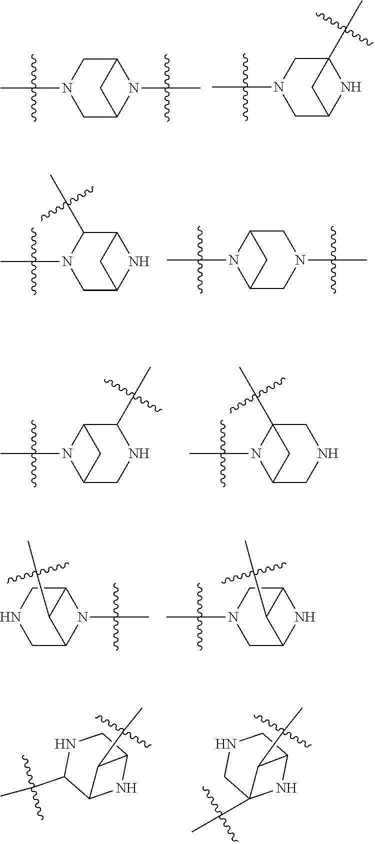 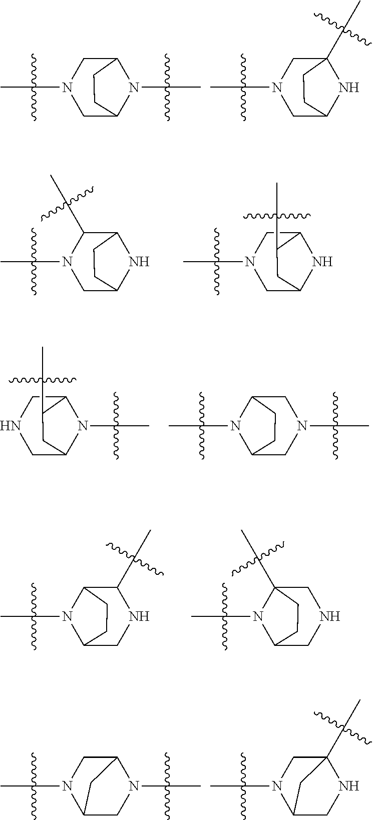 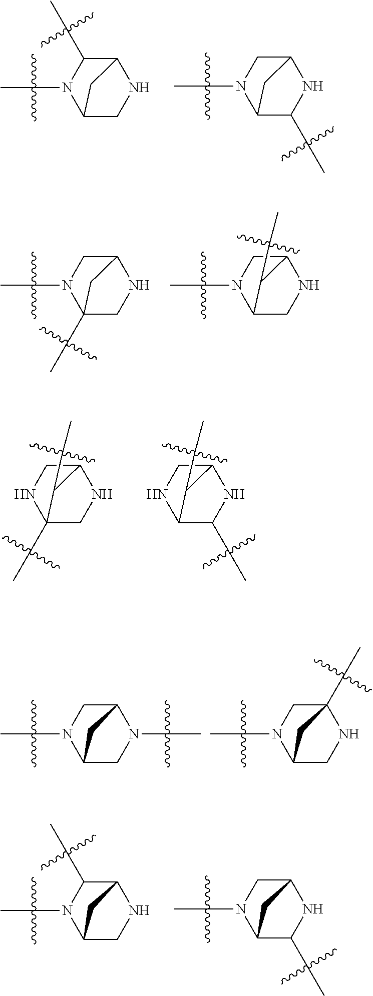 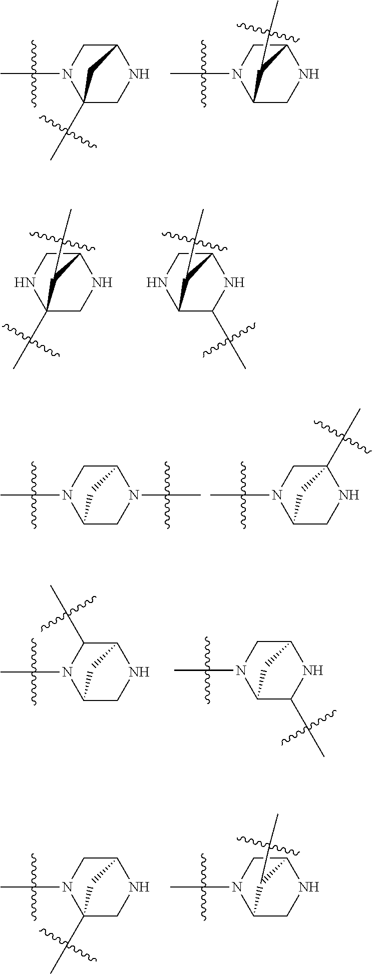 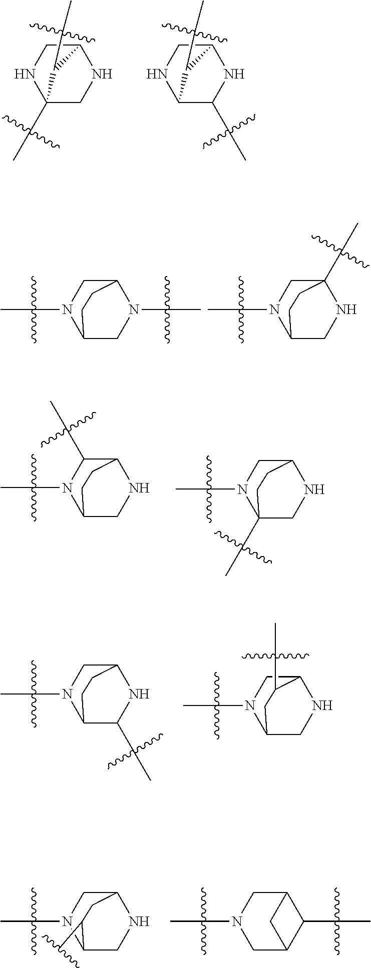 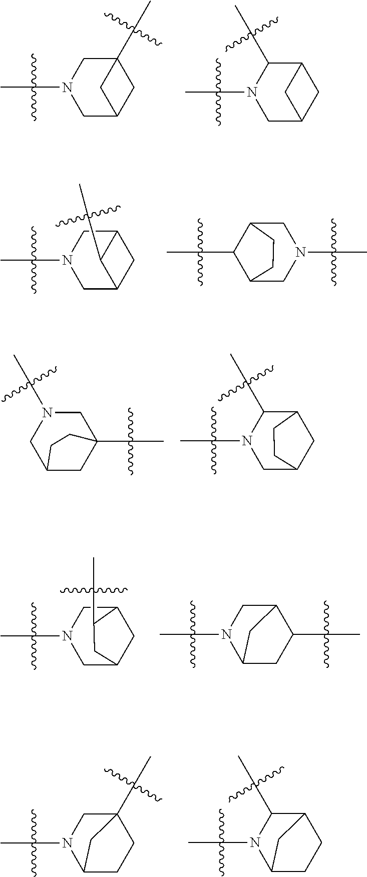 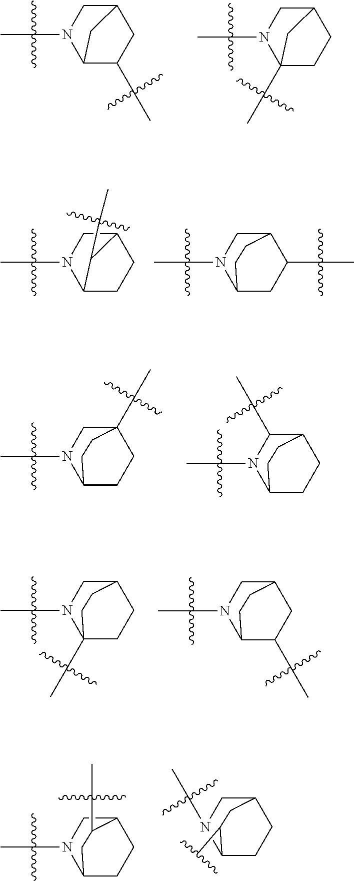 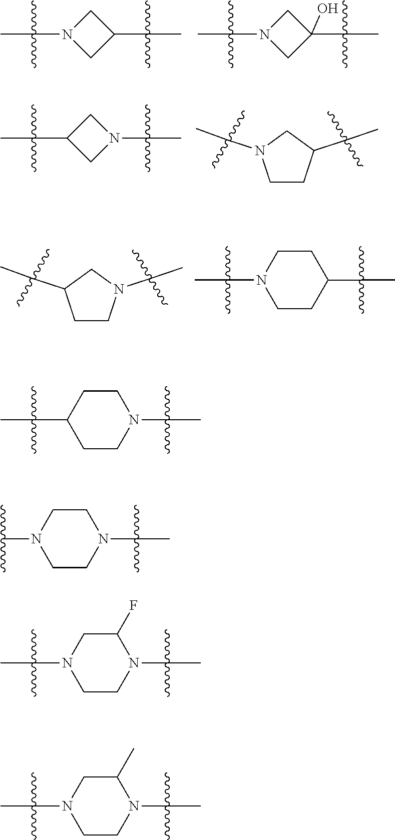 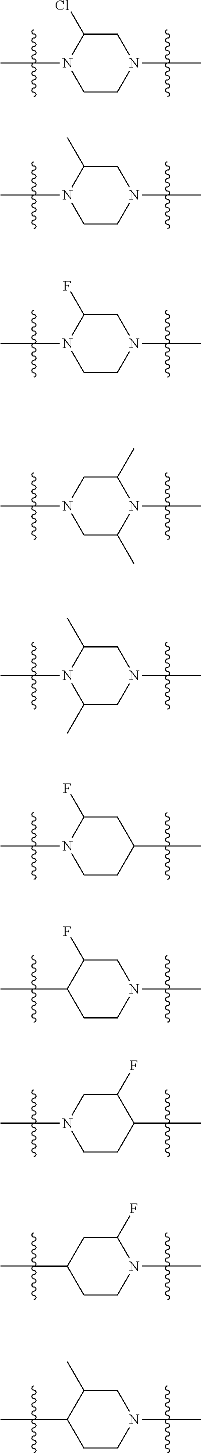 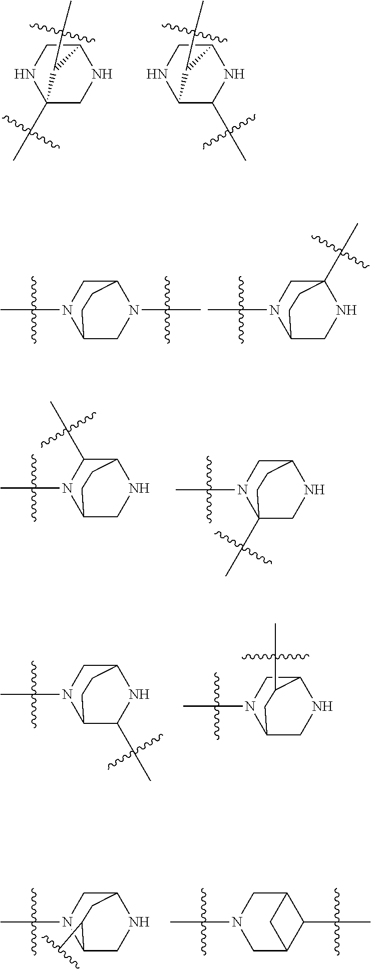 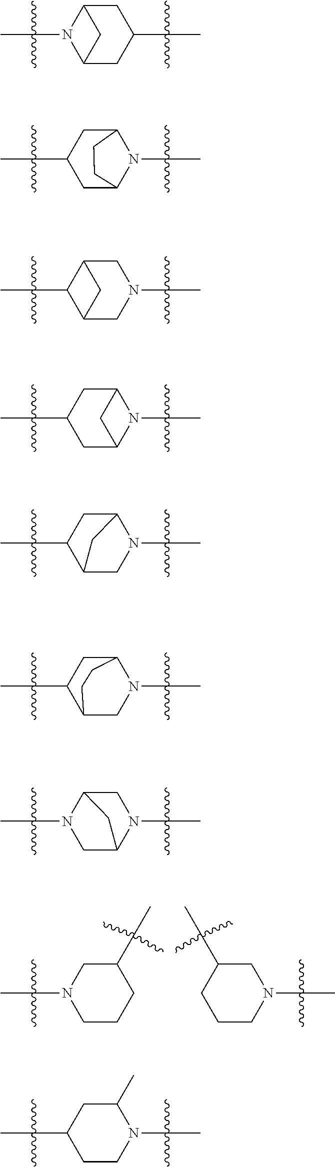 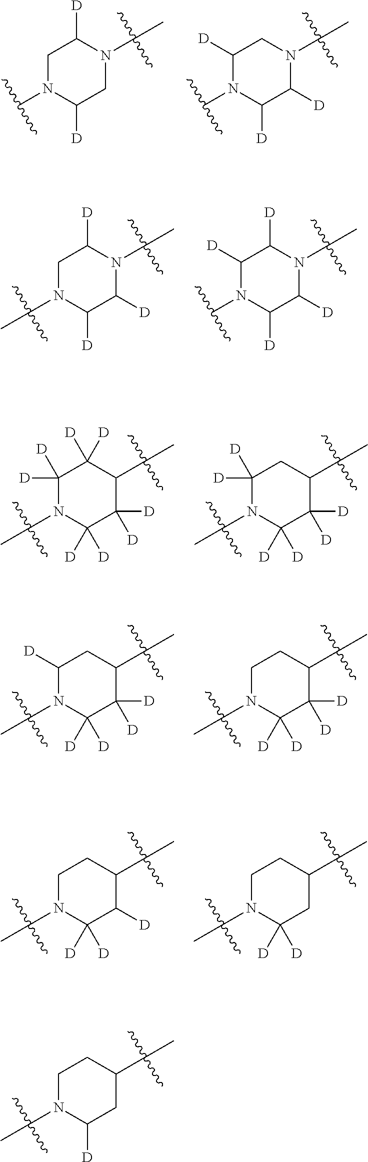 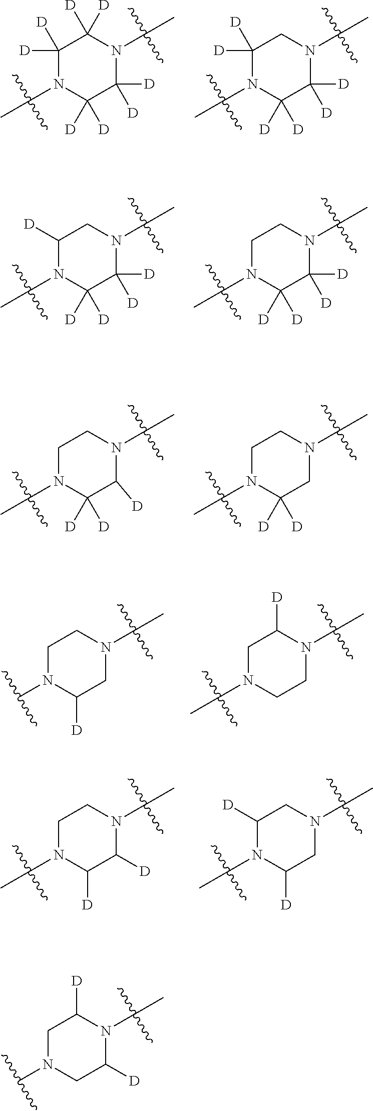 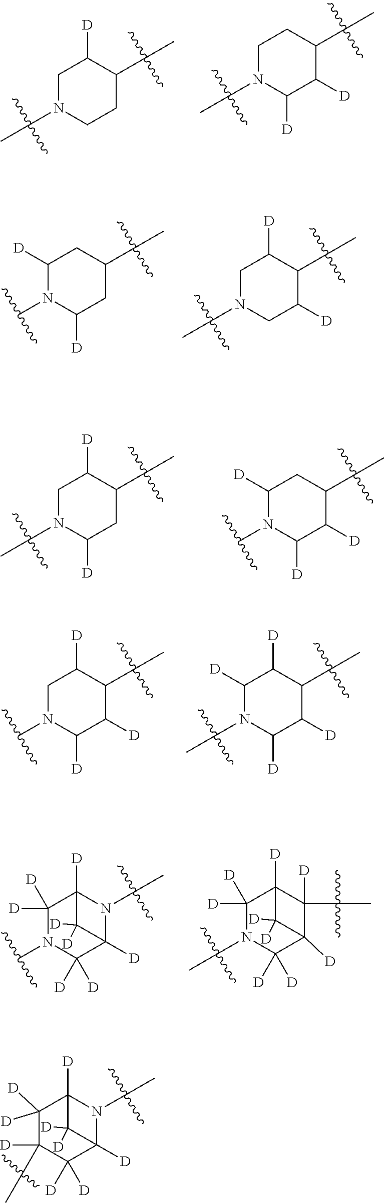 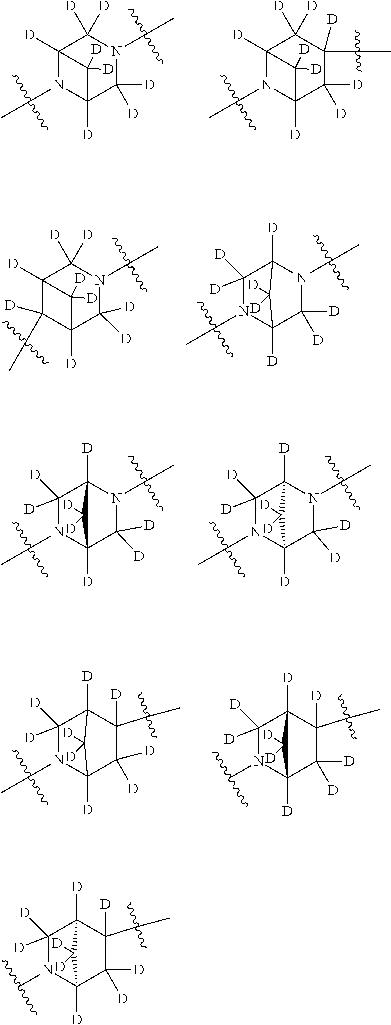 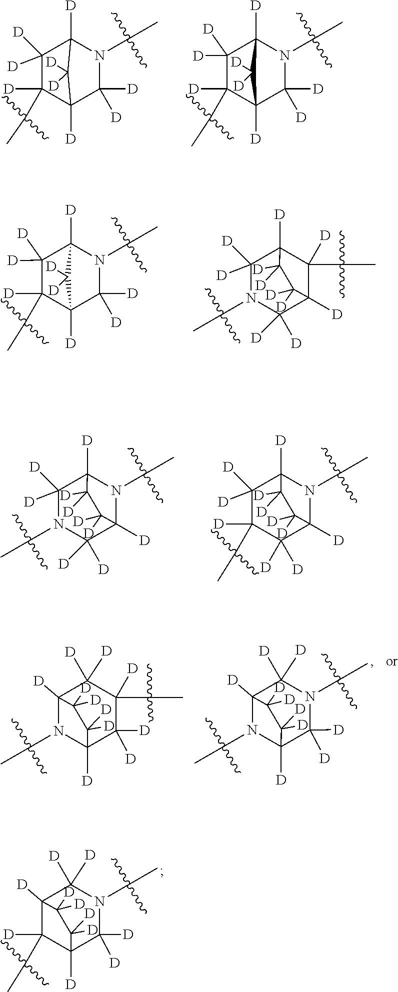
y3 represents an integer of 0, 1, 2, or 3, and (R^{y3})ₐ₃ indicates that each ring A³ is independently optionally substituted with a3 R^{y3} substituents, wherein each R^{y3} independently represents deuterium, C₁₋₆ alkyl, deuterated C₁₋₆ alkyl, halogenated C₁₋₆ alkyl, C₁₋₆ alkoxy, halogen, amino, hydroxy, mercapto, cyano, carboxyl, C₂₋₆ alkynyl, or C₂₋₆ alkenyl, and a3 represents an integer of 0-10, and/or
each ring A⁴ is the same or different and independently represents 4-to 20-membered heterocyclylene, C₃₋₂₀ cycloalkylene, C₅₋₂₀ arylene, or 5-to 20-membered heteroarylene, preferably each ring A⁴ independently represents:
azetidinylene, oxetanylene, pyrrolidinylene, imidazolidinylene, pyrazolidylene, tetrahydrofuranylene, tetrahydropyranylene, tetrahydrothienylene, tetrahydrothiopyranylene, oxazolidinylene, thiazolidinylene, piperidinylene, piperazinylene, morpholinylene, thiomorpholinylene, azacycloheptanylene, azacyclooctylene, dioxacyclohexylene, azacycloheptanylene, azacyclooctylene, diazacycloheptanylene, diazacyclooctylene, 6-azabicyclo[3.1.1]heptanylene, 2,5-diazabicyclo[2.2.1]heptanylene, 3,6-diazabicyclo[3.1.1]heptanylene, 3-azabicyclo[3.2.1]octanylene, 3,8-diazabicyclo[3.2.1]octanylene, 3,8-diazabicyclo[3.2.1]octanylene, 2,5-diazabicyclo[2.2.2]octanylene, 5-to 20-membered azaspirocycloalkylene, cyclopropylene, cyclobutylene, cyclopentylene, cyclopentenylene, cyclohexylene, cyclohexenylene, cycloheptylene, cyclooctylene, decalinylene, octahydropentalenylene, octahydro-1H-indenylene, 2,3-dihydro-1H-indenylene, C₅-15 spiro-cycloalkylene, adamantanylene, noradamantanylene, norbornylene, phenylene, naphthylene, furanylene, oxazolylene, isoxazolylene, oxadiazolylene, thienylene, thiazolylene, isothiazolylene, thiadiazolylene, pyrrolylene, imidazolylene, pyrazolylene, triazolylene, pyridylene, pyrimidinylene, pyridazinylene, pyrazinylene, indolylene, isoindolylene, benzofuranylene, isobenzofuranylene, benzothienylene, indazolylene, benzimidazolylene, benzoxazolylene, benzisoxazolylene, benzothiazolylene, benzisothiazolylene, benzotriazolylene, benzo[2,1,3]oxadiazolylene, benzo[2,1,3]thiadiazolylene, benzo[1,2,3]thiadiazolylene, quinolinylene, isoquinolinylene, naphthyridinylene, cinnolinylene, quinazolinylene, quinoxalinylene, phthalazinylene, pyrazolo[1,5-a]pyridylene, pyrazolo[1,5-a]pyrimidinylene, imidazo[1,2-a]pyridylene, 1H-pyrrolo[3,2-b]pyridylene, 1H-pyrrolo[2,3-b]pyridylene, pyrrolo[2,1-b]thiazolylene, imidazo[2,1-b]thiazolylene, or 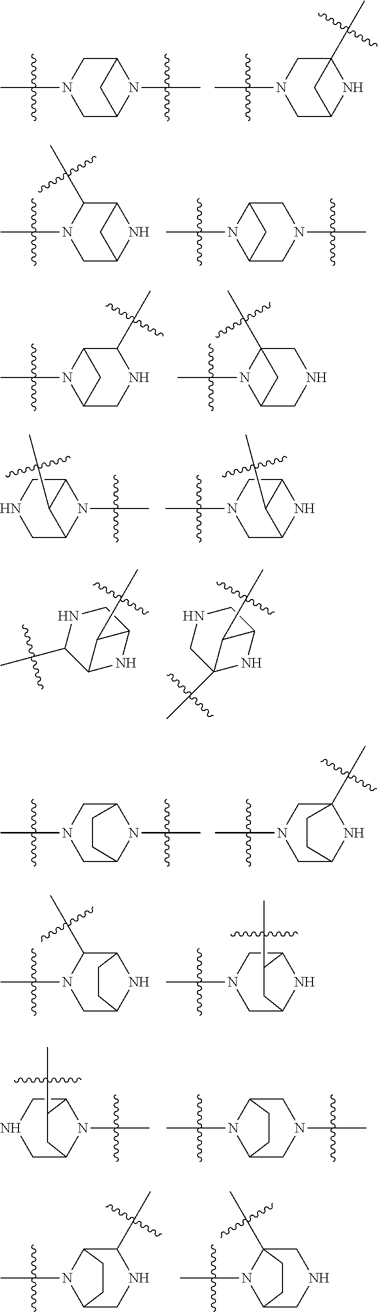 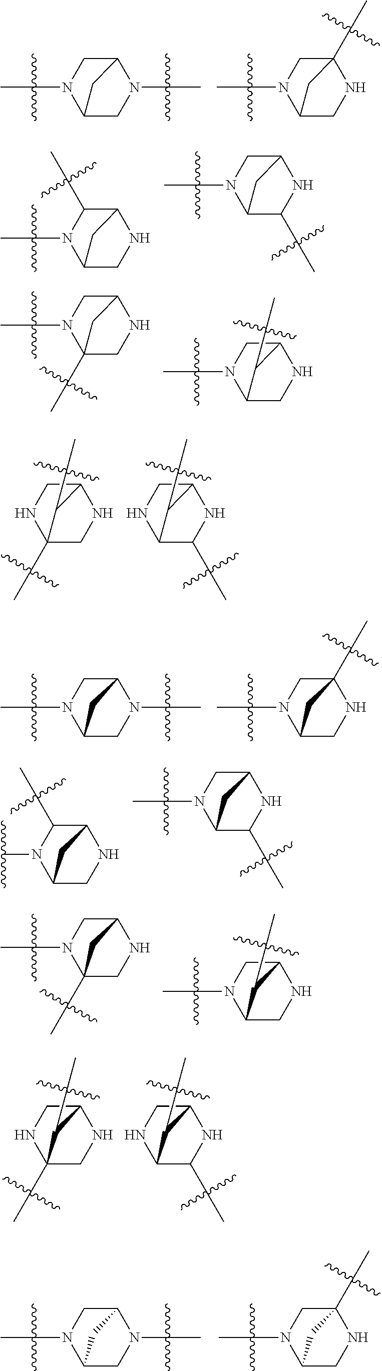 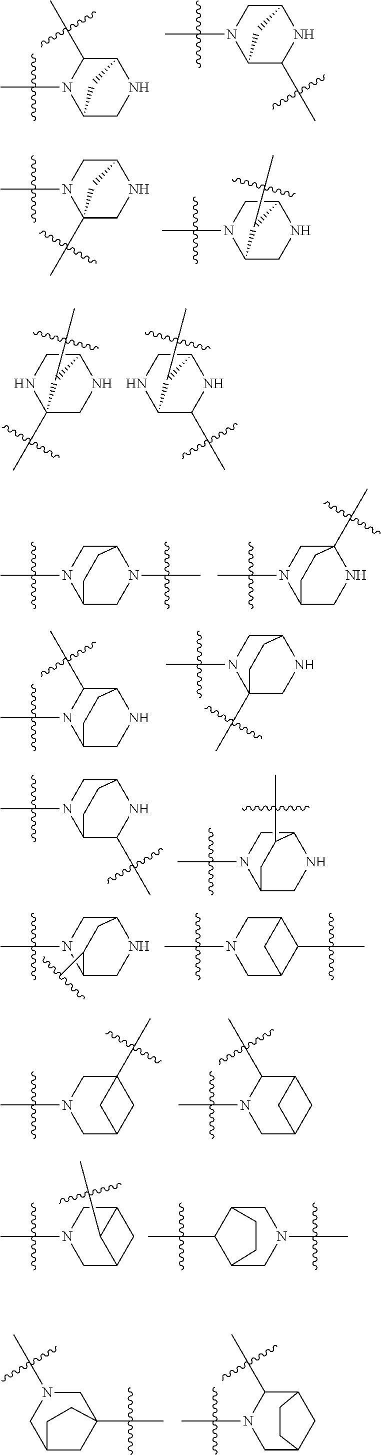 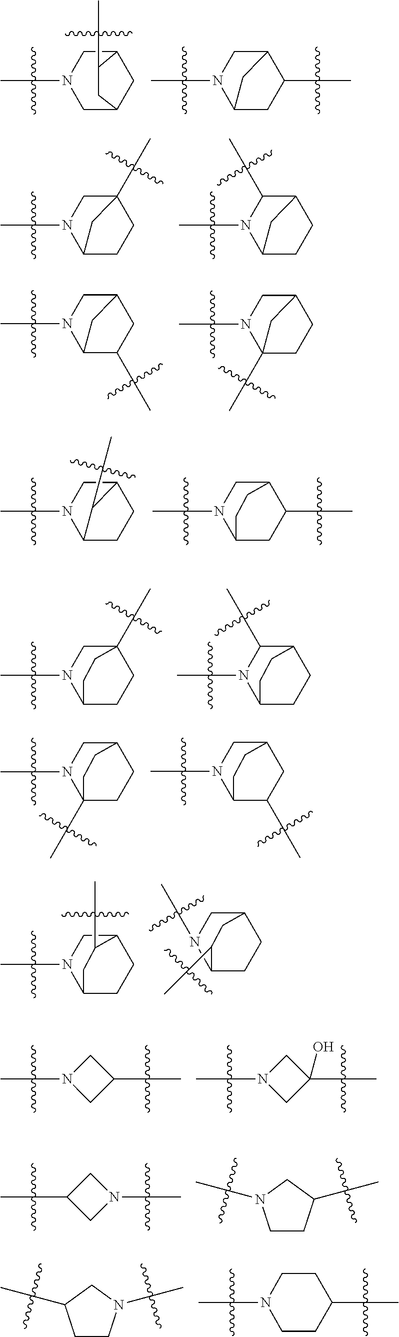 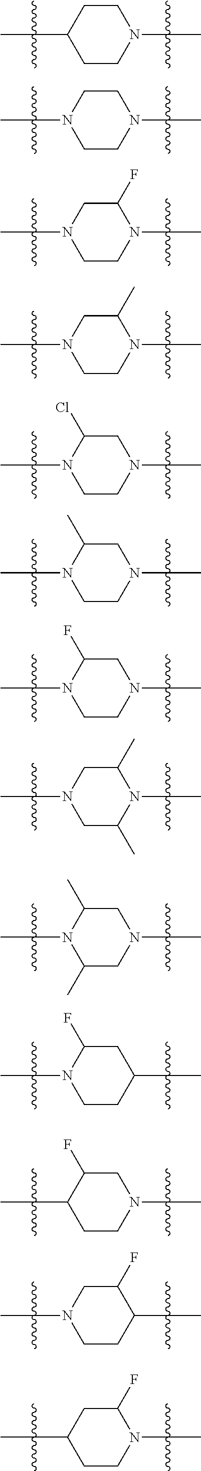 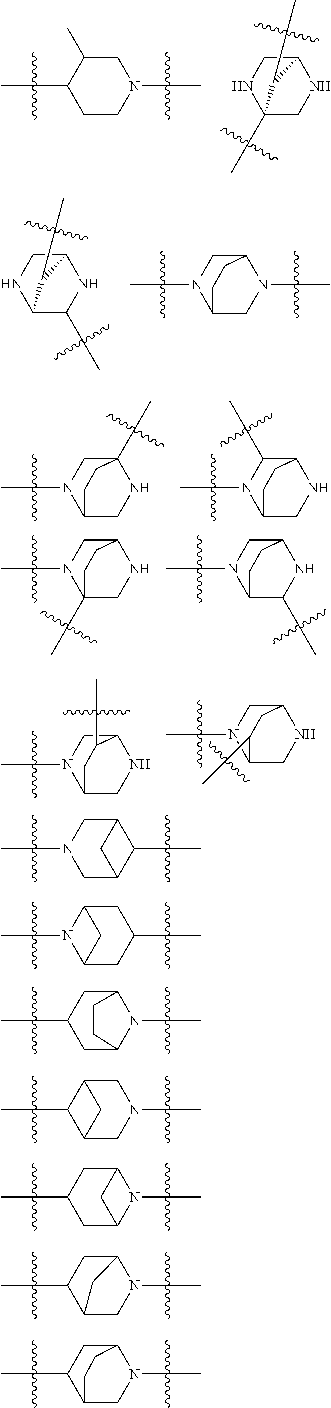 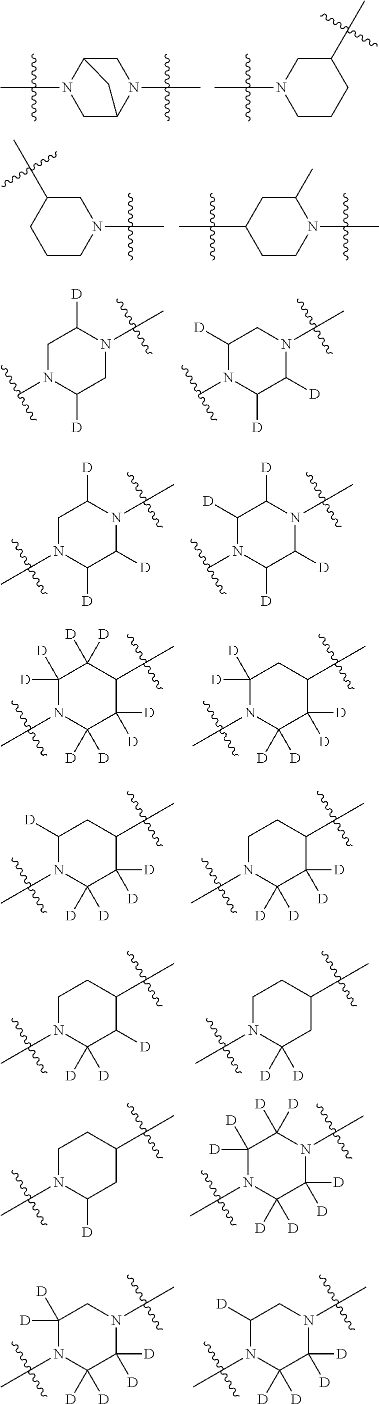 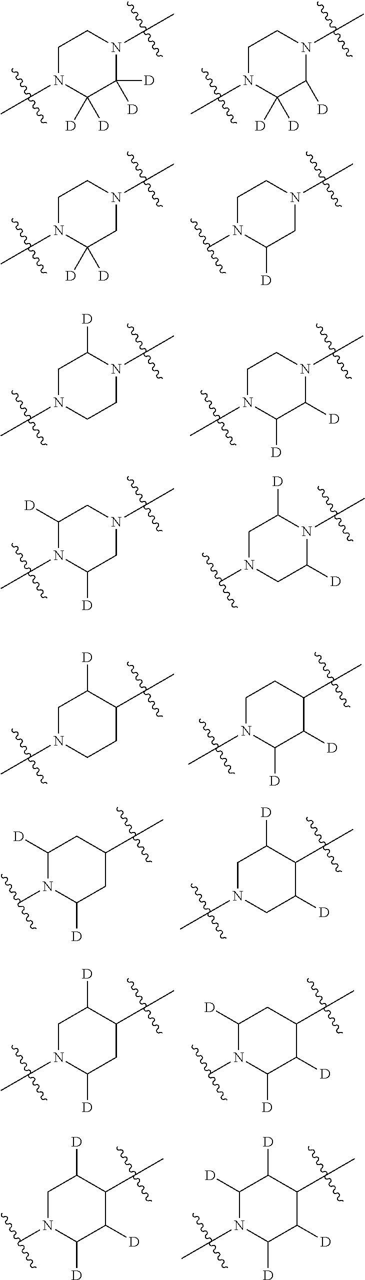 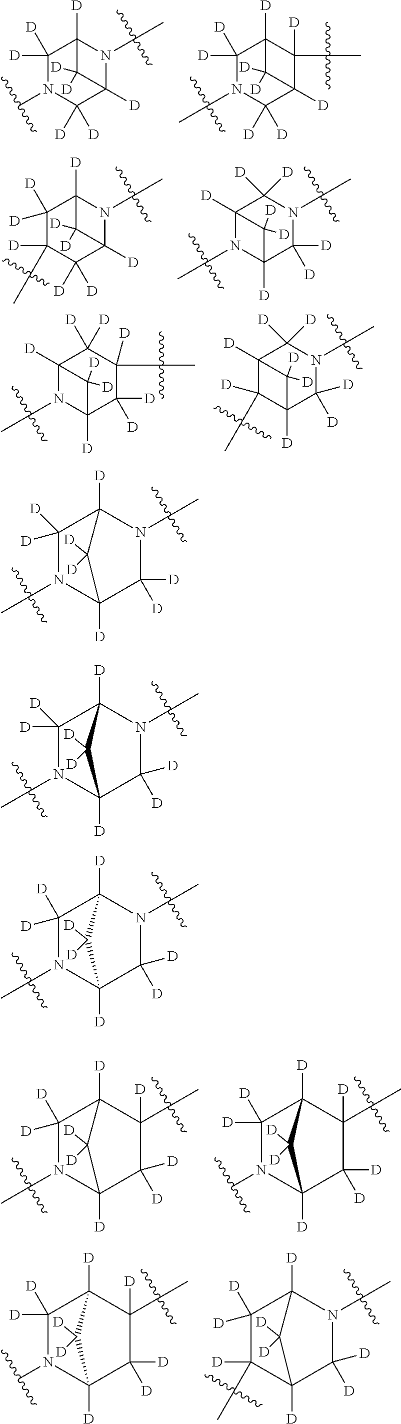 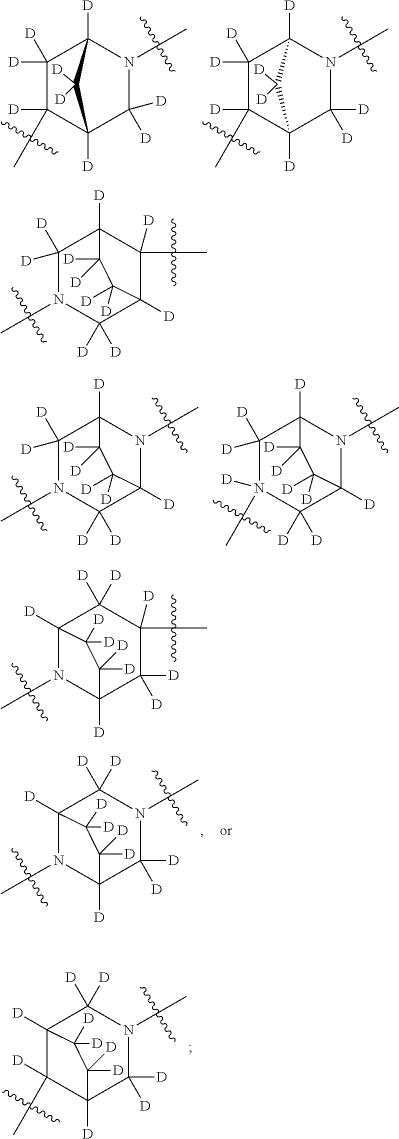
y4 represents an integer of 0, 1, 2, or 3, and (R^{y4}) a4 indicates that each ring A⁴ is independently optionally substituted with a4 R^{y4} substituents, wherein each R^{y4} independently represents deuterium, C₁₋₆ alkyl, deuterated C₁₋₆ alkyl, halogenated C₁₋₆ alkyl, C₁₋₆ alkoxy, halogen, amino, hydroxy, mercapto, cyano, carboxyl, C₂₋₆ alkynyl, or C₂₋₆ alkenyl, and a4 represents an integer of 0-10, and/or
each ring A⁵ is the same or different and independently represents 4-to 20-membered heterocyclylene, C₃₋₂₀cycloalkylene, C₅₋₂₀ arylene, or 5-to 20-membered heteroarylene, preferably each ring A⁵ independently represents:
azetidinylene, oxetanylene, pyrrolidinylene, imidazolidinylene, pyrazolidylene, tetrahydrofuranylene, tetrahydropyranylene, tetrahydrothienylene, tetrahydrothiopyranylene, oxazolidinylene, thiazolidinylene, piperidinylene, piperazinylene, morpholinylene, thiomorpholinylene, azacycloheptanylene, azacyclooctylene, dioxacyclohexylene, azacycloheptanylene, azacyclooctylene, diazacycloheptanylene, diazacyclooctylene, 6-azabicyclo[3.1.1]heptanylene, 2,5-diazabicyclo[2.2.1]heptanylene, 3,6-diazabicyclo[3.1.1]heptanylene, 3-azabicyclo[3.2.1]octanylene, 3,8-diazabicyclo[3.2.1]octanylene, 3,8-diazabicyclo[3.2.1]octanylene, 2,5-diazabicyclo[2.2.2]octanylene, 5-to 20-membered azaspirocycloalkylene, cyclopropylene, cyclobutylene, cyclopentylene, cyclopentenylene, cyclohexylene, cyclohexenylene, cycloheptylene, cyclooctylene, decalinylene, octahydropentalenylene, octahydro-1H-indenylene, 2,3-dihydro-1H-indenylene, C₅-15 spiro-cycloalkylene, adamantanylene, noradamantanylene, norbornylene, phenylene, naphthylene, furanylene, oxazolylene, isoxazolylene, oxadiazolylene, thienylene, thiazolylene, isothiazolylene, thiadiazolylene, pyrrolylene, imidazolylene, pyrazolylene, triazolylene, pyridylene, pyrimidinylene, pyridazinylene, pyrazinylene, indolylene, isoindolylene, benzofuranylene, isobenzofuranylene, benzothienylene, indazolylene, benzimidazolylene, benzoxazolylene, benzisoxazolylene, benzothiazolylene, benzisothiazolylene, benzotriazolylene, benzo[2,1,3]oxadiazolylene, benzo[2,1,3]thiadiazolylene, benzo[1,2,3]thiadiazolylene, quinolinylene, isoquinolinylene, naphthyridinylene, cinnolinylene, quinazolinylene, quinoxalinylene, phthalazinylene, pyrazolo[1,5-a]pyridylene, pyrazolo[1,5-a]pyrimidinylene, imidazo[1,2-a]pyridylene, 1H-pyrrolo[3,2-b]pyridylene, 1H-pyrrolo[2,3-b]pyridylene, pyrrolo[2,1-b]thiazolylene, imidazo[2,1-b]thiazolylene, or 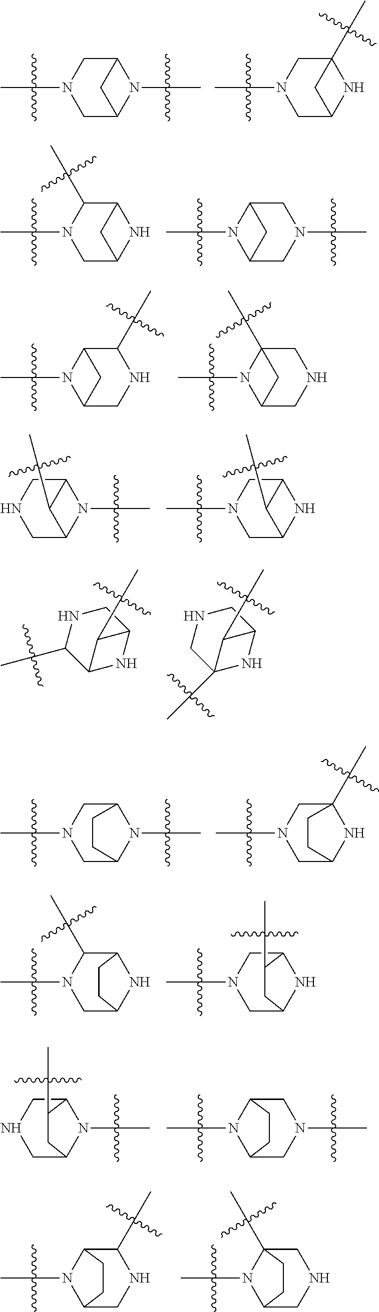 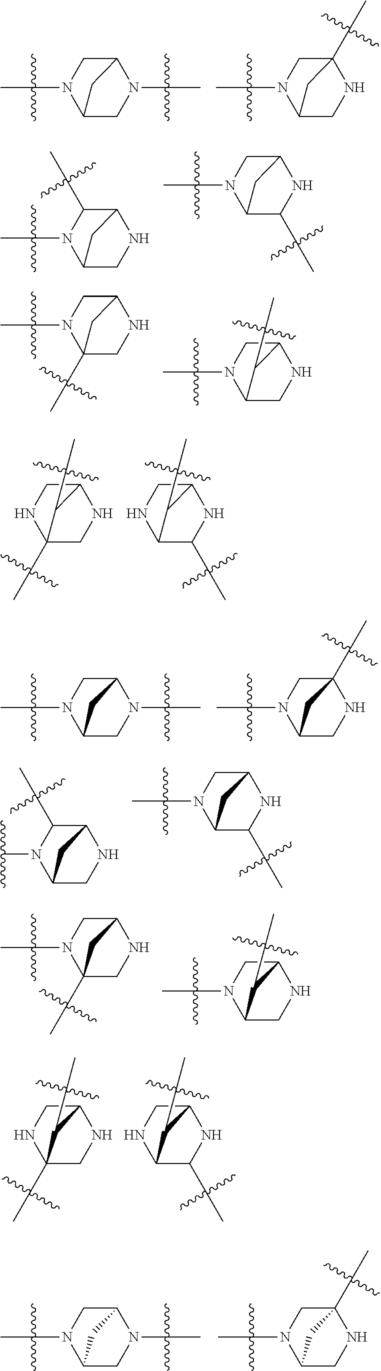 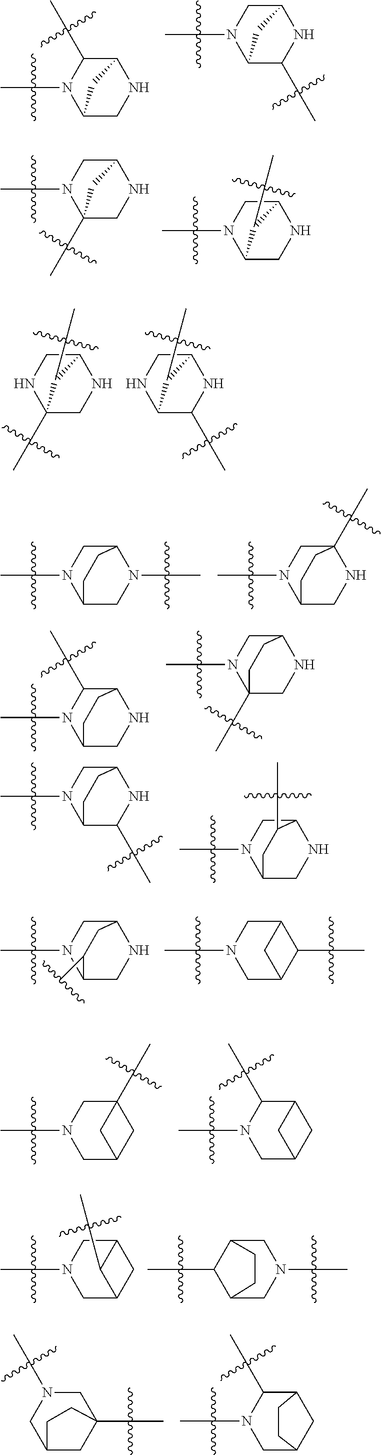 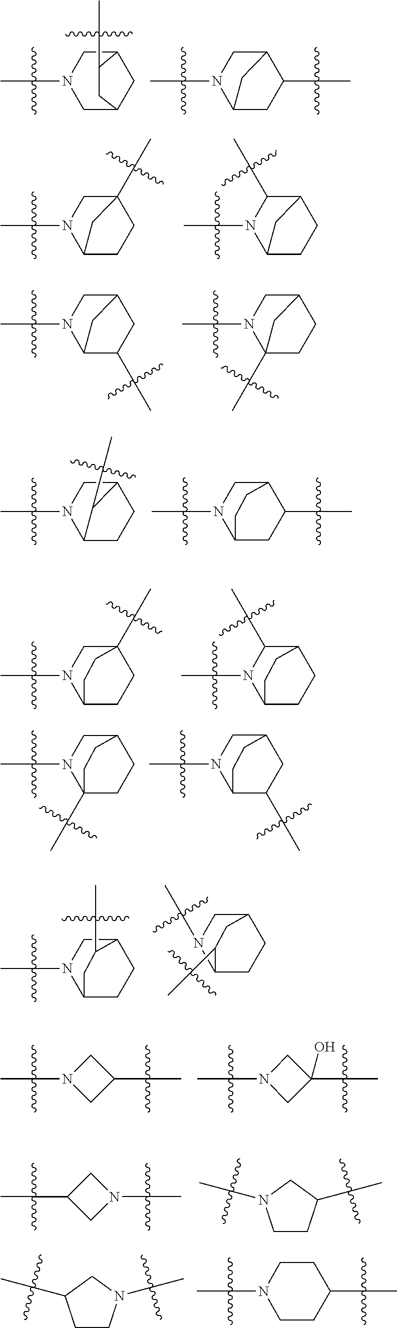 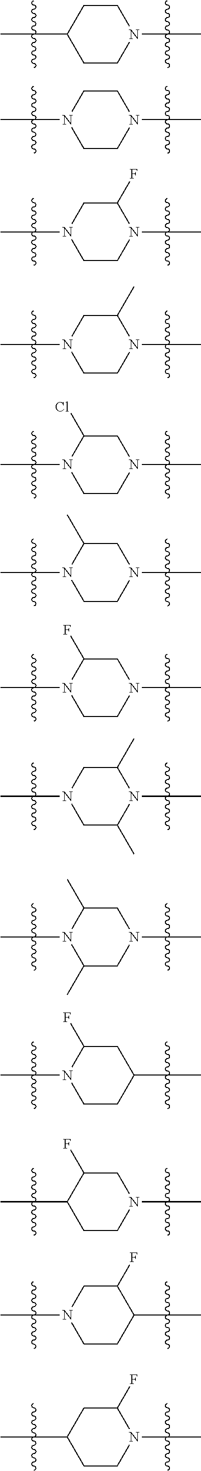 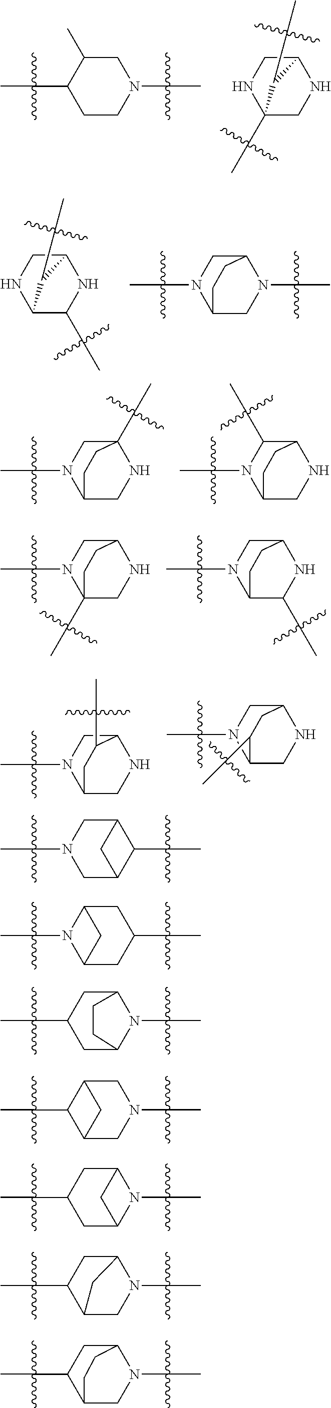 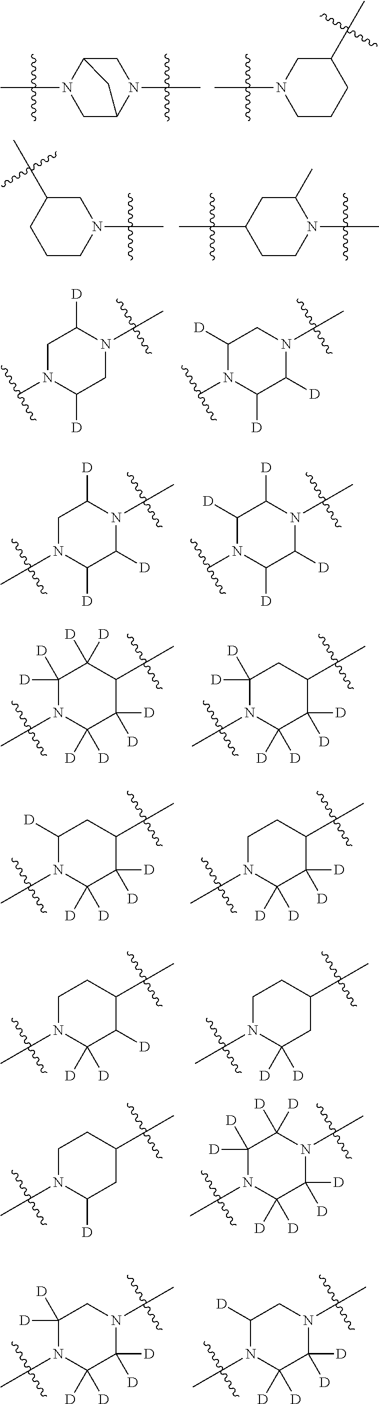 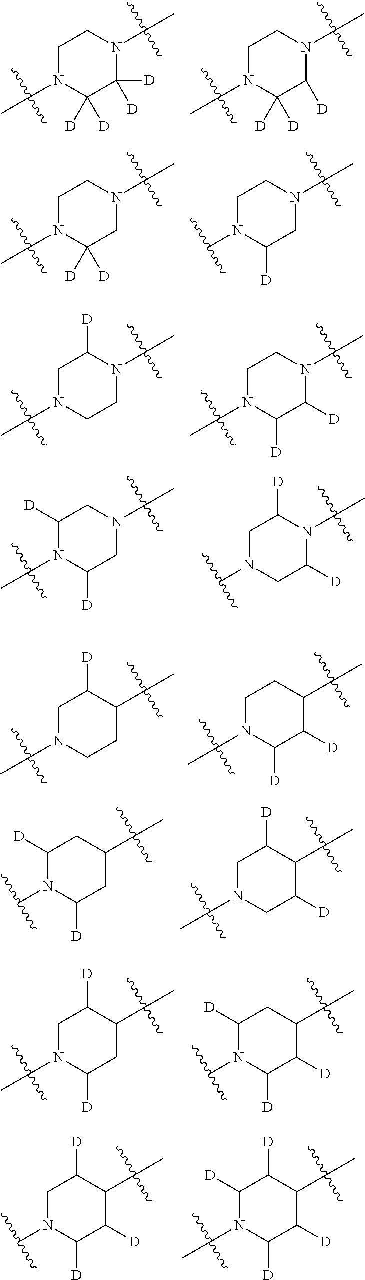 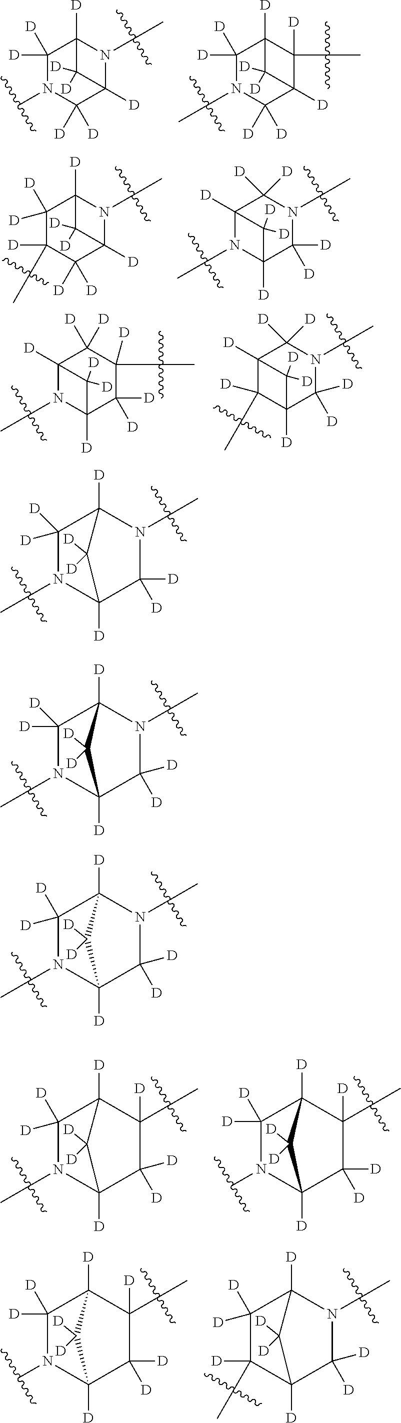 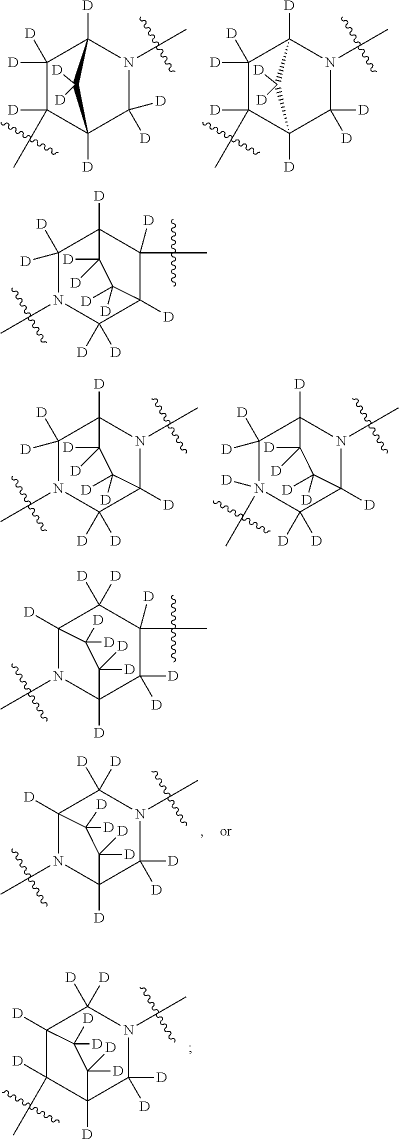
y5 represents an integer of 0, 1, 2, or 3, and (R^{y5}) as indicates that each ring A⁵ is independently optionally substituted with a5 R^{y5} substituents, wherein each R^{y5} independently represents deuterium, C₁₋₆ alkyl, deuterated C₁₋₆ alkyl, halogenated C₁₋₆ alkyl, C₁₋₆ alkoxy, halogen, amino, hydroxy, mercapto, cyano, carboxyl, C₂₋₆ alkynyl, or C₂₋₆ alkenyl, and a5 represents an integer of 0-10, and/or
R_{w1} represents a bond or optionally substituted linear or branched C₁₋₂₀ alkylene, wherein any one or more methylene groups in the main carbon chain skeleton of the linear or branched C₁₋₂₀ alkylene are optionally replaced by one or more groups selected from Rₐ, R_{b}, and any combination thereof; wherein each R^{a} is independently selected from the group consisting of: O, C(O), OC(O), S, S(O), S(O)₂, S(O)₂N(Rₐ₇), N(Rₐ₇)S(O)₂, C(O)N(Rₐ₇), N(Rₐ₇)C(O), N(Rₐ₇), and N(Rₐ₇)C(O)N(Rₐ₇), where each Rₐ₇ is independently H or C₁₋₃ alkyl, and when any two or more methylene in the main carbon chain skeleton of the linear or branched C₁₋₂₀ alkylene are replaced by two or more Rₐ groups, the Rₐ groups are not directly connected to each other; and wherein each R^{b} is independently selected from the group consisting of: optionally substituted cycloalkylene (e.g., optionally substituted C₃₋₂₀ cycloalkylene), optionally substituted arylene (e.g., optionally substituted C₅₋₂₀ arylene), optionally substituted heterocyclylene (e.g., optionally substituted 4-to 20-membered heterocyclylene), optionally substituted heteroarylene (e.g., optionally substituted 5-to 20-membered heteroarylene), alkynylene (e.g., C₂₋₆ alkynylene), alkenylene (e.g., C₂₋₆ alkenylene), and any combination thereof, wherein the cycloalkylene, arylene, heterocyclylene, and heteroarylene are each independently optionally substituted with a substituent selected from the group consisting of deuterium, hydroxy, amino, mercapto, nitro, halogen, cyano, oxo, C₁₋₆ alkyl, halogenated C₁₋₆ alkyl, deuterated C₁₋₆ alkyl, C₁₋₆ alkoxy, halogenated C₁₋₆ alkoxy, C₃₋₆ cycloalkyl, halogenated C₃₋₆ cycloalkyl, deuterated C₃₋₆ cycloalkyl, 4-to 10-membered heterocyclyl, and any combination thereof, and wherein one or more hydrogens of said linear or branched C₁₋₂₀ alkylene are optionally replaced by a substituent selected from the group consisting of deuterium, hydroxy, amino, mercapto, nitro, halogen, cyano, halogenated C₁₋₆ alkyl, deuterated C₁₋₆ alkyl, C₁₋₆ alkoxy, halogenated C₁₋₆ alkoxy, C₃₋₆ cycloalkyl, halogenated C₃₋₆ cycloalkyl, deuterated C₃₋₆ cycloalkyl, 4-to 10-membered heterocyclyl, and any combination thereof.

14. The compound of Formula (I) or salts, enantiomers, stereoisomers, solvates, isotopically enriched analogs, prodrugs, or polymorphs thereof as claimed in claim 1, wherein the 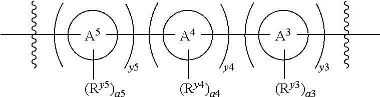
moiety in the general formula of LIN represents: 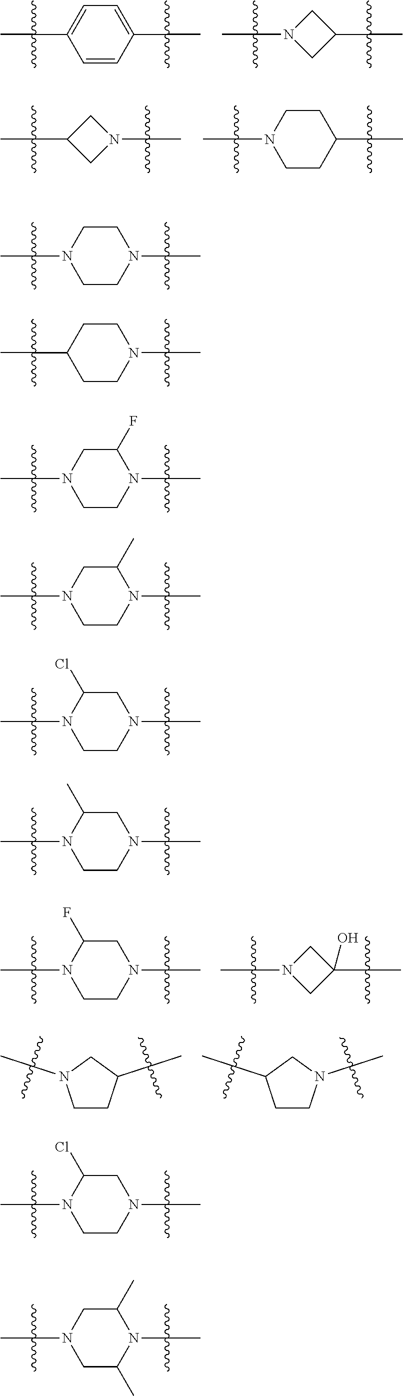 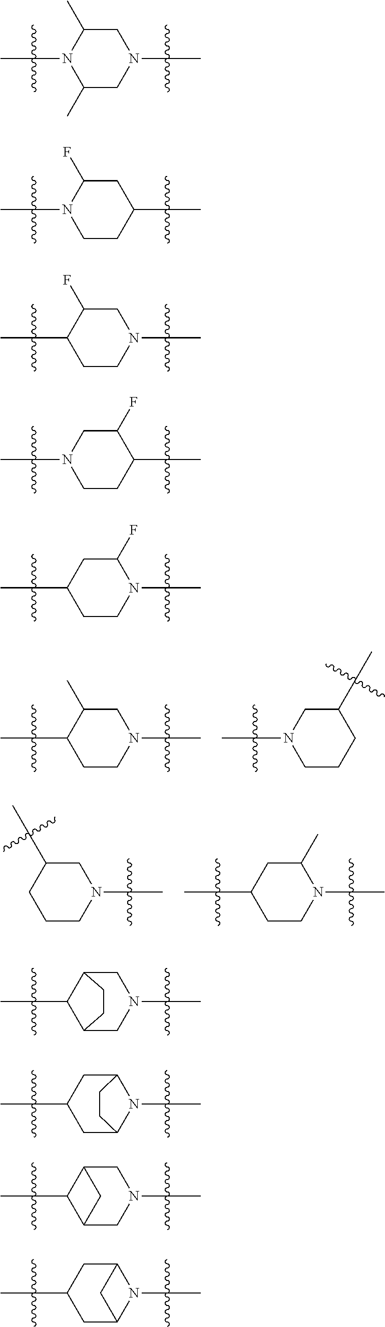 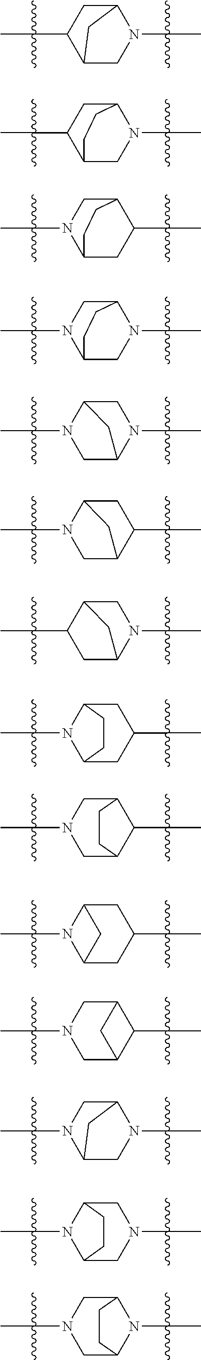 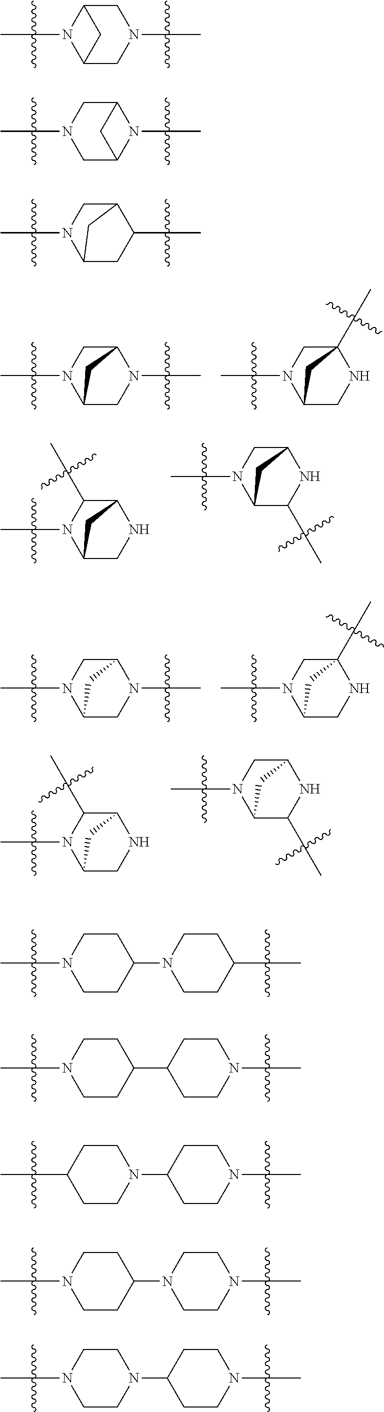 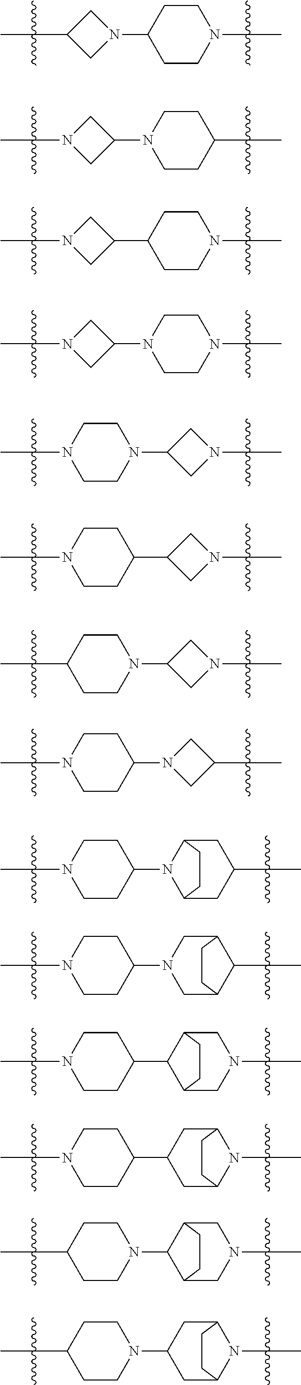 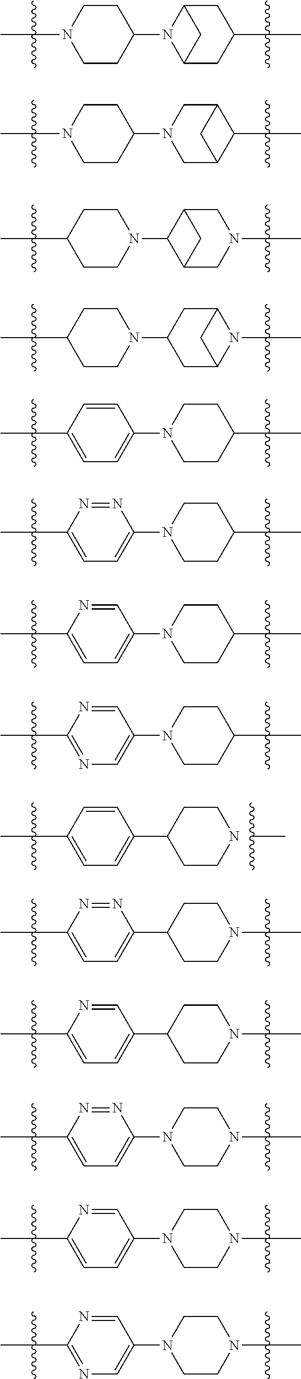 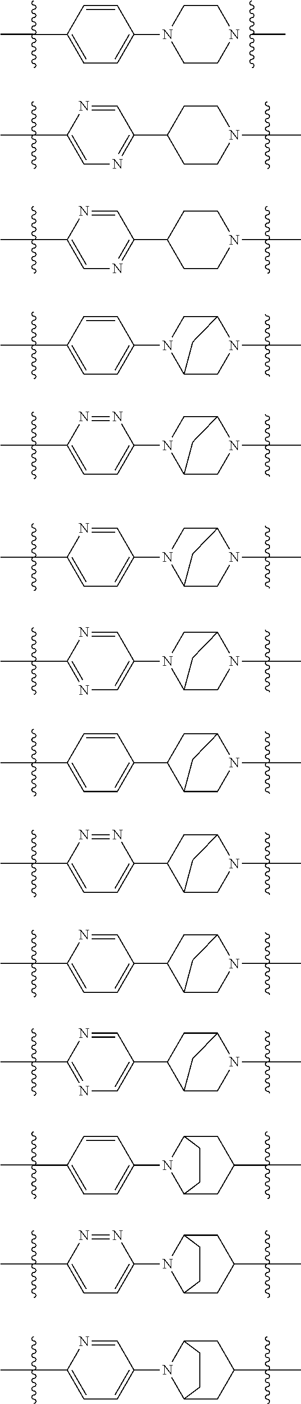 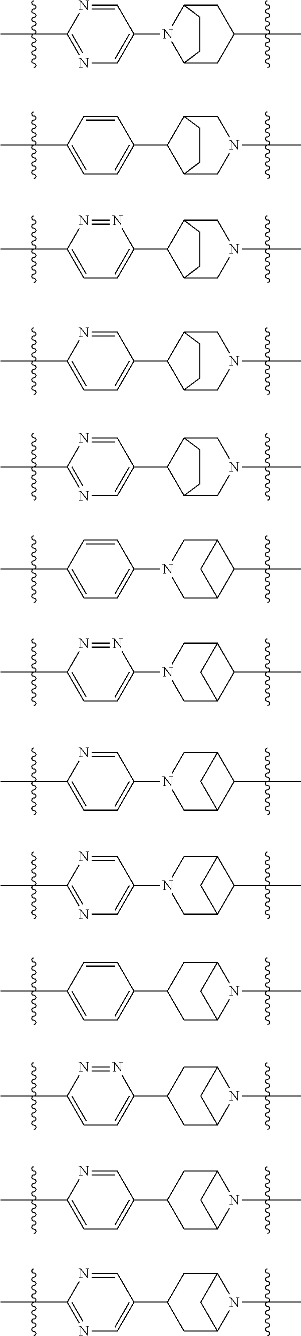 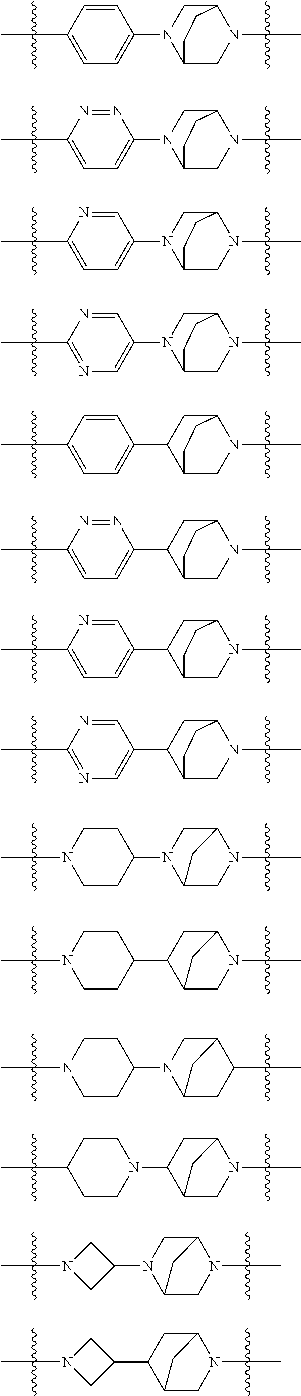 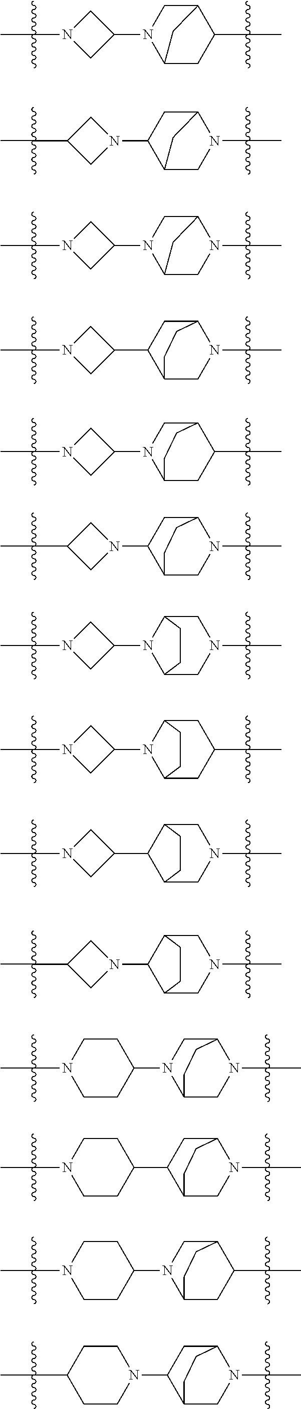 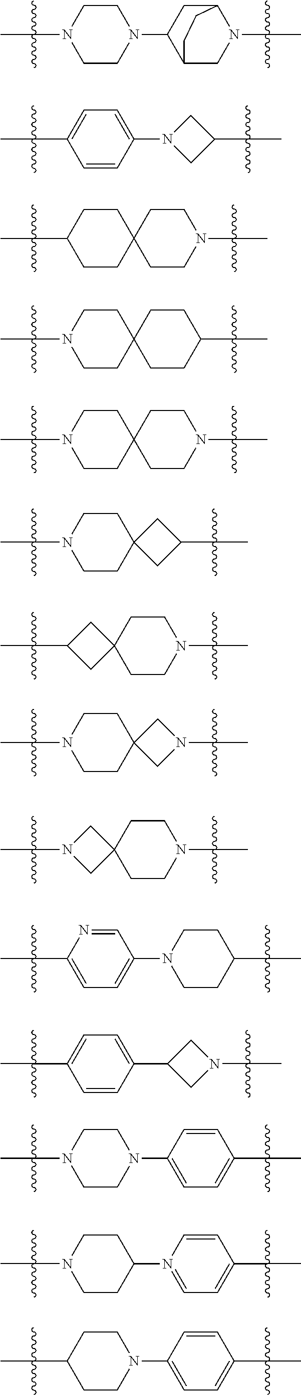 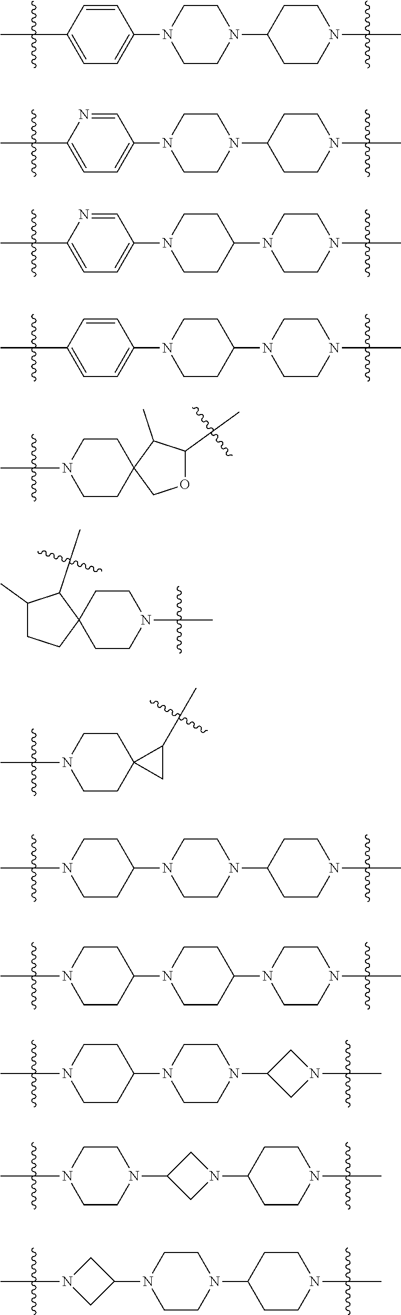 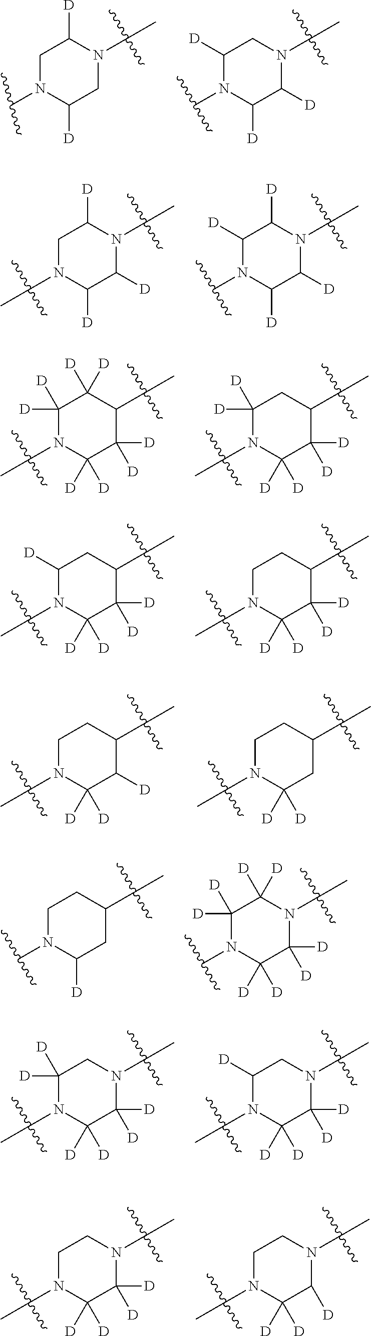 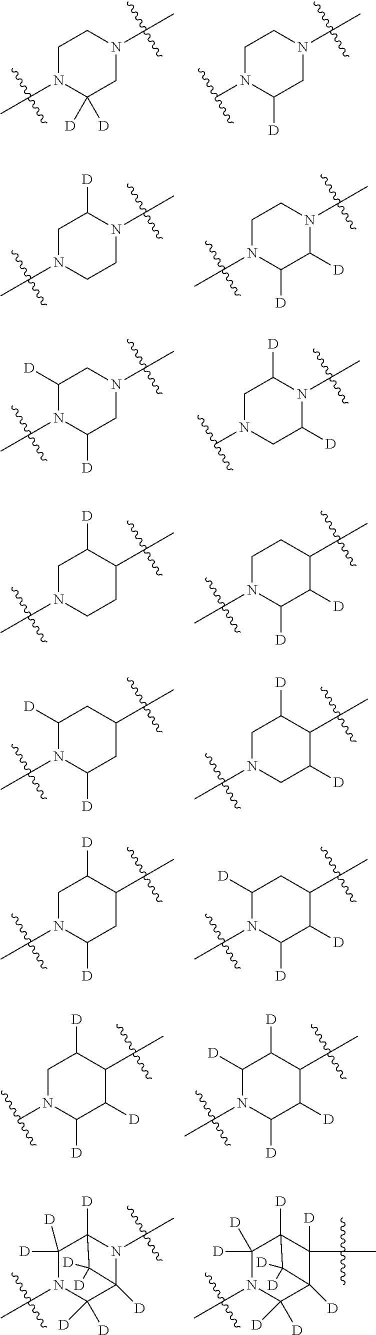 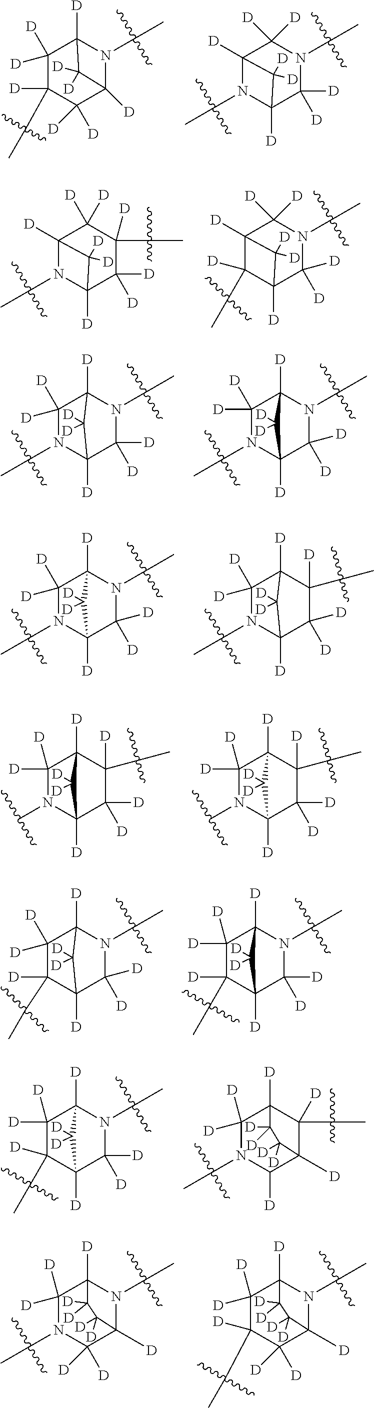 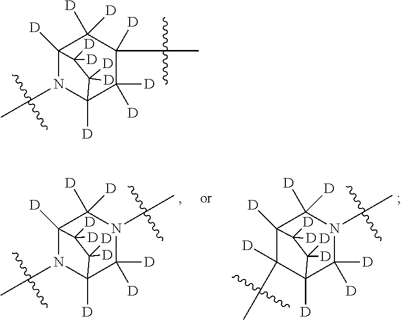
wherein the groups are optionally substituted with a substituent selected from the group consisting of deuterium, C₁₋₆ alkyl, deuterated C₁₋₆ alkyl, halogenated C₁₋₆ alkyl, C₁₋₆ alkoxy, halogen, amino, hydroxy, mercapto, cyano, carboxyl, C₂₋₆ alkynyl, or C₂₋₆ alkenyl, and/or
R_{w1} represents a bond, or
R_{w1} represents:
—C₁₋₁₅ alkylene-;
—(C(Rₐ₈)(Rₐ₉))ₙ₁—(Rₐ(C(Rₐ₁₀)(Rₐ₁₁))ₙ₂)ₘ₃—;
—(C(Rₐ₈)(Rₐ₉))ₙ₁—(Rₐ(C(Rₐ₁₀)(Rₐ₁₁))ₙ₂)ₘ₃—(Rₐ(C(Rₐ₁₂)(Rₐ₁₃))ₙ₃)ₘ₄—;
—(C(Rₐ₈)(Rₐ₉))ₙ₁—(Rₐ(C(Rₐ₁₀)(Rₐ₁₁))ₙ₂)ₘ₃—(Rₐ(C(Rₐ₁₂)(Rₐ₁₃))ₙ₃)ₘ₄—(Rₐ(C(Rₐ₁₄)(Rₐ₁₅))ₙ₄)ₘ₅—;
—(C(Rₐ₅)(Rₐ))ₙ₁—((C(Rₐ₁₀)(Rₐ₁₁))ₙ₂Rₐ)ₘ₃—;
—(C(Rₐ₈)(Rₐ₉))ₙ₁—((C(Rₐ₁₀)(Rₐ₁₁))ₙ₂Rₐ)ₘ₃—((C(Rₐ₁₂)(Rₐ₁₃))ₙ₃Rₐ)ₘ₄—;
—(C(Rₐ₈)(Rₐ₉))ₙ₁—((C(Rₐ₁₀)(Rₐ₁₁))ₙ₂Rₐ)ₘ₃—((C(Rₐ₁₂)(Rₐ₁₃))ₙ₃Rₐ)ₘ₄—((C(Rₐ₁₄)(Rₐ₁₅))ₙ₄Rₐ)ₘ₅—;
—(C(Rₐ₈)(Rₐ₉))ₙ₁—(R_{b}(C(Rₐ₁₀)(Rₐ₁₁))ₙ₂)ₘ₃—;
—(C(Rₐ₈)(Rₐ₉))ₙ₁—(R_{b}(C(Rₐ₁₀)(Rₐ₁₁))ₙ₂)ₘ₃—(R_{b}(C(Rₐ₁₂)(Rₐ₁₃))ₙ₃)ₘ₄—;
—(C(Rₐ₈)(Rₐ₉))ₙ₁—(R_{b}(C(Rₐ₁₀)(Rₐ₁₁))ₙ₂)ₘ₃—(R_{b}(C(Rₐ₁₂)(Rₐ₁₃))ₙ₃)ₘ₄—(R_{b}(C(Rₐ₁₄)(Rₐ₁₅))ₙ₄)ₘ₅—;
—(C(Rₐ₈)(Rₐ₉))ₙ₁—((C(Rₐ₁₀)(Rₐ₁₁))ₙ₂R_{b})ₘ₃—;
—(C(Rₐ₈)(Rₐ₉))ₙ₁—((C(Rₐ₁₀)(Rₐ₁₁))ₙ₂R_{b})ₘ₃—((C(Rₐ₁₂)(Rₐ₁₃))ₙ₃R_{b})ₘ₄—;
—(C(Rₐ₈)(Rₐ₉))ₙ₁—((C(Rₐ₁₀)(Rₐ₁₁))ₙ₂R_{b})ₘ₃—((C(Rₐ₁₂)(Rₐ₁₃))ₙ₃R_{b})ₘ₄—((C(Rₐ₁₄)(Rₐ₁₅))ₙ₄R_{b})ₘ₅—;
—(C(Rₐ₈)(Rₐ₉))ₙ₁—(Rₐ—R_{b}—(C(Rₐ₁₀)(Rₐ₁₁))ₙ₂)ₘ₃—;
—(C(Rₐ₈)(Rₐ₉))ₙ₁—(Rₐ(C(Rₐ₁₀)(Rₐ₁₁))ₙ₂)ₘ₃—(R_{b}(C(Rₐ₁₂)(Rₐ₁₃))ₙ₃)ₘ₄—;
—(C(Rₐ₈)(Rₐ₉))ₙ₁—(R_{b}—Rₐ—(C(Rₐ₁₀)(Rₐ₁₁))ₙ₂)ₘ₃—;
—(C(Rₐ₈)(Rₐ₉))ₙ₁—(R_{b}(C(Rₐ₁₀)(Rₐ₁₁))ₙ₂)ₘ₃—(Rₐ(C(Rₐ₁₂)(Rₐ₁₃))ₙ₃)ₘ₄—;
—(C(Rₐ₈)(Rₐ₉))ₙ₁—((C(Rₐ₁₀)(Rₐ₁₁))ₙ₂—Rₐ—R_{b})ₘ₃—;
—(C(Rₐ₈)(Rₐ₉))ₙ₁—((C(Rₐ₁₀)(Rₐ₁₁))ₙ₂Rₐ)ₘ₃—((C(Rₐ₁₂)(Rₐ₁₃))ₙ₃R_{b})ₘ₄—;
—(C(Rₐ₈)(Rₐ₉))ₙ₁—((C(Rₐ₁₀)(Rₐ₁₁))ₙ₂—R_{b}—Rₐ)ₘ₃—, or
—(C(Rₐ₈)(Rₐ₉))ₙ₁—((C(Rₐ₁₀)(Rₐ₁₁))ₙ₂R_{b})ₘ₃—((C(Rₐ₁₂)(Rₐ₁₃))ₙ₃Rₐ)ₘ₄—;
wherein each R^{a} is independently selected from the group consisting of O, C(O), OC(O), S, S(O), S(O)₂, S(O)₂N(Rₐ₇), N(Rₐ₇)S(O)₂, C(O)N(Rₐ₇), N(Rₐ₇)C(O), N(Rₐ₇), and N(Rₐ₇)C(O)N(Rₐ₇), wherein each Rₐ₇ independently represents H or C₁₋₃ alkyl; and each R^{b} is independently selected from the group consisting of optionally substituted cycloalkylene (e.g., optionally substituted C₃₋₂₀ cycloalkylene), optionally substituted arylene (e.g., optionally substituted C₅₋₂₀ arylene), optionally substituted heterocyclylene (e.g., optionally substituted 4-to 20-membered heterocyclylene), optionally substituted heteroarylene (e.g., 5-to 20-membered heteroarylene), alkynylene (e.g., C₂₋₆alkynylene), alkenylene (e.g., C₂₋₆alkenylene), and any combination thereof, wherein the cycloalkylene, arylene, heterocyclylene, and heteroarylene are each independently optionally substituted with a substituent selected from the group consisting of deuterium, hydroxy, amino, mercapto, nitro, halogen, cyano, oxo, C₁₋₆ alkyl, halogenated C₁₋₆ alkyl, deuterated C₁₋₆ alkyl, C₁₋₆ alkoxy, halogenated C₁₋₆ alkoxy, C₃₋₆ cycloalkyl, halogenated C₃₋₆ cycloalkyl, deuterated C₃₋₆ cycloalkyl, 4-to 10-membered heterocyclyl, and any combination thereof;
Rₐ₈, Rₐ₉, Rₐ₁₀, Rₐ₁₁, Rₐ₁₂, Rₐ₁₃, Rₐ₁₄, and Rₐ₁₅ each independently represent H, deuterium, hydroxy, amino, mercapto, nitro, halogen, cyano, oxo, halogenated C₁₋₆ alkyl, deuterated C₁₋₆ alkyl, C₁₋₆ alkoxy, halogenated C₁₋₆ alkoxy, C₃₋₆ cycloalkyl, halogenated C₃₋₆ cycloalkyl, deuterated C₃₋₆ cycloalkyl, 4-to 10-membered heterocyclyl, and any combination thereof, and
n1, n2, n3, n4, m3, m4, m5 each independently represent an integer of 1, 2, 3, 4, 5, 6, 7, 8, 9, or 10; and
any one or more hydrogens of said C₁₋₁₅ alkylene are optionally replaced by a substituent selected from: deuterium, hydroxy, amino, mercapto, nitro, halogen, cyano, oxo, halogenated C₁₋₆ alkyl, deuterated C₁₋₆ alkyl, C₁₋₆ alkoxy, halogenated C₁₋₆ alkoxy, C₃₋₆ cycloalkyl, halogenated C₃₋₆ cycloalkyl, deuterated C₃₋₆ cycloalkyl, 4-to 10-membered heterocyclyl, and any combination thereof.

15. The compound of Formula (I) or salts, enantiomers, stereoisomers, solvates, isotopically enriched analogs, prodrugs, or polymorphs thereof as claimed in claim 1, wherein the R_{w1} represents a structure of the following Formulae:
—(C(Rₐ₈)(Rₐ₉))ₙ₁—(O(C(Rₐ₁₀)(Rₐ₁₁))ₙ₂)ₘ₃—;
—(C(Rₐ₈)(Rₐ₉))ₙ₁—(O(C(Rₐ₁₀)(Rₐ₁₁))ₙ₂)ₘ₃—(O(C(Rₐ₁₂)(Rₐ₁₃))ₙ₃)ₘ₄—;
—(C(Rₐ₈)(Rₐ₉))ₙ₁—(O(C(Rₐ₁₀)(Rₐ₁₁))ₙ₂)ₘ₃—(O(C(Rₐ₁₂)(Rₐ₁₃))ₙ₃)ₘ₄—(O(C(Rₐ₁₄)(Rₐ₁₅))ₙ₄)ₘ₅—;
—(C(Rₐ₈)(Rₐ₉))ₙ₁—((C(Rₐ₁₀)(Rₐ₁₁))ₙ₂O)ₘ₃—;
—(C(Rₐ₈)(Rₐ₉))ₙ₁—((C(Rₐ₁₀)(Rₐ₁₁))ₙ₂O)ₘ₃—((C(Rₐ₁₂)(Rₐ₁₃))ₙ₃₀)ₘ₄—;
—(C(Rₐ₈)(Rₐ₉))ₙ₁—((C(Rₐ₁₀)(Rₐ₁₁))ₙ₂O)ₘ₃—((C(Rₐ₁₂)(Rₐ₁₃))ₙ₃₀)ₘ₄—((C(Rₐ₁₄)(Rₐ₁₅))ₙ₄₀)ₘ₅—;
—(C(Rₐ₈)(Rₐ₉))ₙ₁—(N(Rₐ₇)(C(Rₐ₁₀)(Rₐ₁₁))ₙ₂)ₘ₃—;
—(C(Rₐ₈)(Rₐ₉))ₙ₁—(N(Rₐ₇)(C(Rₐ₁₀)(Rₐ₁₁))ₙ₂)ₘ₃—(N(Rₐ₇)(C(Rₐ₁₂)(Rₐ₁₃))ₙ₃)ₘ₄—;
—(C(Rₐ₈)(Rₐ₉))ₙ₁—(N(Rₐ₇)(C(Rₐ₁₀)(Rₐ₁₁))ₙ₂)ₘ₃—(N(Rₐ₇)(C(Rₐ₁₂)(Rₐ₁₃))ₙ₃)ₘ₄—(N(Rₐ₇)(C(Rₐ₁₄)(Rₐ₁₅))ₙ₄)ₘ₅—;
—(C(Rₐ₈)(Rₐ₉))ₙ₁—((C(Rₐ₁₀)(Rₐ₁₁))ₙ₂N(Rₐ₇))ₘ₃—;
—(C(Rₐ₈)(Rₐ₉))ₙ₁—((C(Rₐ₁₀)(Rₐ₁₁))ₙ₂N(Rₐ₇))ₘ₃—((C(Rₐ₁₂)(Rₐ₁₃))ₙ₃N(Rₐ₇))ₘ₄—;
—(C(Rₐ₈)(Rₐ₉))ₙ₁—((C(Rₐ₁₀)(Rₐ₁₁))ₙ₂N(Rₐ₇))ₘ₃—((C(Rₐ₁₂)(Rₐ₁₃))ₙ₃N(Rₐ₇))ₘ₄—((C(Rₐ₁₄)(Rₐ₁₅))ₙ₄N(Rₐ₇))ₘ₅—;
—(C(Rₐ₈)(Rₐ₉))ₙ₁—(C(O)N(Rₐ₇)—(C(Rₐ₁₀)(Rₐ₁₁))ₙ₂)ₘ₃—;
—(C(Rₐ₈)(Rₐ₉))ₙ₁—(C(O)N(Rₐ₇)—(C(Rₐ₁₀)(Rₐ₁₁))ₙ₂)ₘ₃—(C(O)N(Rₐ₇)—(C(Rₐ₁₂)(Rₐ₁₃))ₙ₃)ₘ₄—;
—(C(Rₐ₈)(Rₐ₉))ₙ₁—(C(O)N(Rₐ₇)—(C(Rₐ₁₀)(Rₐ₁₁))ₙ₂)ₘ₃—(C(O)N(Rₐ₇)—(C(Rₐ₁₂)(Rₐ₁₃))ₙ₃)ₘ₄—(C(O)N(Rₐ₇)—(C(Rₐ₁₄)(Rₐ₁₅))ₙ₄)ₘ₅—;
—(C(Rₐ₈)(Rₐ₉))ₙ₁—((C(Rₐ₁₀)(Rₐ₁₁))ₙ₂—C(O)N(Rₐ₇))ₘ₃—;
—(C(Rₐ₈)(Rₐ₉))ₙ₁—((C(Rₐ₁₀)(Rₐ₁₁))ₙ₂—C(O)N(Rₐ₇))ₘ₃—((C(Rₐ₁₂)(Rₐ₁₃))ₙ₃—C(O)N(Rₐ₇))ₘ₄—;
—(C(Rₐ₈)(Rₐ₉))ₙ₁—((C(Rₐ₁₀)(Rₐ₁₁))ₙ₂—C(O)N(Rₐ₇))ₘ₃—((C(Rₐ₁₂)(Rₐ₁₃))ₙ₃—C(O)N(Rₐ₇))ₘ₄—((C(Rₐ₁₄)(Rₐ₁₅))ₙ₄—C(O)N(Rₐ₇))ₘ₅—;
—(C(Rₐ₈)(Rₐ₉))ₙ₁—(C(O)N(Rₐ₇)—(C(Rₐ₁₀)(Rₐ₁₁))ₙ₂)ₘ₃—(O)—(C(Rₐ₁₂)(Rₐ₁₃))ₙ₃)ₘ₄—;
—(C(Rₐ₈)(Rₐ₉))ₙ₁—C(O)N(Rₐ₇)—(C(Rₐ₁₀)(Rₐ₁₁))ₙ₂—(O(C(Rₐ₁₂)(Rₐ₁₃))ₙ₃)ₘ₃—;
—(C(Rₐ₈)(Rₐ₉))ₙ₁—((C(Rₐ₁₀)(Rₐ₁₁))ₙ₂—C(O)N(Rₐ₇))ₘ₃—((C(Rₐ₁₂)(Rₐ₁₃))ₙ₃O)ₘ₄—;
—(C(Rₐ₈)(Rₐ₉))ₙ₁—(C(Rₐ₁₀)(Rₐ₁₁))ₙ₂—C(O)N(Rₐ₇)—((C(Rₐ₁₂)(Rₐ₁₃))ₙ₃₀)ₘ₃—;
—(C(Rₐ₈)(Rₐ₉))ₙ₁—(N(Rₐ₇)C(O)—(C(Rₐ₁₀)(Rₐ₁₁))ₙ₂)ₘ₃—;
—(C(Rₐ₈)(Rₐ₉))ₙ₁—(N(Rₐ₇)C(O)—(C(Rₐ₁₀)(Rₐ₁₁))ₙ₂)ₘ₃—(O—(C(Rₐ₁₂)(Rₐ₁₃)))ₙ₃)ₘ₄—;
—(C(Rₐ₈)(Rₐ₉))ₙ₁—(N(Rₐ₇)C(O)—(C(Rₐ₁₀)(Rₐ₁₁))ₙ₂)ₘ₃—(O—(C(Rₐ₁₂)(Rₐ₁₃))ₙ₃)ₘ₄—(O—(C(Rₐ₁₄)(Rₐ₁₅))ₙ₄)ₘ₅—;
—(C(Rₐ₈)(Rₐ₉))ₙ₁—N(Rₐ₇)C(O)—(C(Rₐ₁₀)(Rₐ₁₁))ₙ₂—(O(C(Rₐ₁₂)(Rₐ₁₃))ₙ₃)ₘ₃—;
—(C(Rₐ₈)(Rₐ₉))ₙ₁—N(Rₐ₇)C(O)N(Rₐ₇)—(C(Rₐ₁₀)(Rₐ₁₁))ₙ₂—;
—(C(Rₐ₈)(Rₐ₉))ₙ₁—((C(Rₐ₁₀)(Rₐ₁₁))ₙ₂—N(Rₐ₇)C(O))ₘ₃—;
—(C(Rₐ₈)(Rₐ₉))ₙ₁—((C(Rₐ₁₀)(Rₐ₁₁))ₙ₂—N(Rₐ₇)C(O))ₘ₃—((C(Rₐ₁₂)(Rₐ₁₃)))ₙ₃—O)ₘ₄—;
—(C(Rₐ₈)(Rₐ₉))ₙ₁—((C(Rₐ₁₀)(Rₐ₁₁))ₙ₂—N(Rₐ₇)C(O))ₘ₃—((C(Rₐ₁₂)(Rₐ₁₃))ₙ₃₀)ₘ₄—((C(Rₐ₁₄)(Rₐ₁₅))ₙ₄₀)ₘ₅—;
—(C(Rₐ₈)(Rₐ₉))ₙ₁—(C(Rₐ₁₀)(Rₐ₁₁))ₙ₂—N(Rₐ₇)C(O)—((C(Rₐ₁₂)(Rₐ₁₃))ₙ₃₀)ₘ₃—;
—(C(Rₐ₈)(Rₐ₉))ₙ₁-(arylene-(C(Rₐ₁₀)(Rₐ₁₁))ₙ₂)ₘ₃—;
—(C(Rₐ₈)(Rₐ₉))ₙ₁-(arylene-(C(Rₐ₁₀)(Rₐ₁₁))ₙ₂)ₘ₃-arylene-(C(Rₐ₁₂)(Rₐ₁₃))ₙ₃—;
—(C(Rₐ₈)(Rₐ₉))ₙ₁—((C(Rₐ₁₀)(Rₐ₁₁))ₙ₂-arylene)ₘ₃-;
—(C(Rₐ₈)(Rₐ₉))ₙ₁—((C(Rₐ₁₀)(Rₐ₁₁))ₙ₂-arylene)ₘ₃-(C(Rₐ₁₂)(Rₐ₁₃))ₙ₃-arylene-;
—(C(Rₐ₈)(Rₐ₉))ₙ₁-(heterocyclylene-(C(Rₐ₁₀)(Rₐ₁₁))ₙ₂)ₘ₃—;
—(C(Rₐ₈)(Rₐ₉))ₙ₁-(heterocyclylene-(C(Rₐ₁₀)(Rₐ₁₁))ₙ₂)ₘ₃-(heterocyclylene-(C(Rₐ₁₂)(Rₐ₁₃))ₙ₃)ₘ₄—;
—(C(Rₐ₈)(Rₐ₉))ₙ₁—((C(Rₐ₁₀)(Rₐ₁₁))ₙ₂-heterocyclylene)ₘ₃-;
—(C(Rₐ₈)(Rₐ₉))ₙ₁—((C(Rₐ₁₀)(Rₐ₁₁))ₙ₂-heterocyclylene)ₘ₃-((C(Rₐ₁₂)(Rₐ₁₃))ₙ₃-heterocyclylene)ₘ₄-;
—(C(Rₐ₈)(Rₐ₉))ₙ₁-(heteroarylene-(C(Rₐ₁₀)(Rₐ₁₁))ₙ₂)ₘ₃—;
—(C(Rₐ₈)(Rₐ₉))ₙ₁-(heteroarylene-(C(Rₐ₁₀)(Rₐ₁₁))ₙ₂)ₘ₃-(heteroarylene-(C(Rₐ₁₂)(Rₐ₁₃))ₙ₃)ₘ₄—;
—(C(Rₐ₈)(Rₐ₉))ₙ₁—((C(Rₐ₁₀)(Rₐ₁₁))ₙ₂-heteroarylene)ₘ₃-;
—(C(Rₐ₈)(Rₐ₉))ₙ₁—((C(Rₐ₁₀)(Rₐ₁₁))ₙ₂-heteroarylene)ₘ₃-((C(Rₐ₁₂)(Rₐ₁₃))ₙ₃-heteroarylene)ₘ₄-;
—(C(Rₐ₈)(Rₐ₉))ₙ₁-(cycloalkylene-(C(Rₐ₁₀)(Rₐ₁₁))ₙ₂)ₘ₃—;
—(C(Rₐ₈)(Rₐ₉))ₙ₁-(cycloalkylene-(C(Rₐ₁₀)(Rₐ₁₁))ₙ₂)ₘ₃-(cycloalkylene-(C(Rₐ₁₂)(Rₐ₁₃))ₙ₃)ₘ₄—;
—(C(Rₐ₈)(Rₐ₉))ₙ₁—((C(Rₐ₁₀)(Rₐ₁₁))ₙ₂-cycloalkylene)ₘ₃-, or
(C(Rₐ₈)(Rₐ₉))ₙ₁—((C(Rₐ₁₀)(Rₐ₁₁))ₙ₂-cycloalkylene)ₘ₃-((C(Rₐ₁₂)(Rₐ₁₃))ₙ₃-cycloalkylene)ₘ₄-;
wherein each Rₐ₇ independently represents H or C₁₋₃ alkyl;
the cycloalkylene (e.g., C₃₋₂₀cycloalkylene), arylene (e.g., C₅₋₂₀ arylene), heterocyclylene (e.g., 4-to 20-membered heterocyclylene), and heteroarylene (e.g., 5-to 20-membered heteroarylene) are each independently optionally substituted with a substituent selected from the group consisting of deuterium, hydroxy, amino, mercapto, nitro, halogen, cyano, oxo, C₁₋₆ alkyl, halogenated C₁₋₆ alkyl, deuterated C₁₋₆ alkyl, C₁₋₆ alkoxy, halogenated C₁₋₆ alkoxy, C₃₋₆ cycloalkyl, halogenated C₃₋₆ cycloalkyl, deuterated C₃₋₆ cycloalkyl, 4-to 10-membered heterocyclyl, and any combination thereof;
Rₐ₈, Rₐ₉, Rₐ₁₀, Rₐ₁₁, Rₐ₁₂, Rₐ₁₃, Rₐ₁₄, and Rₐ₁₅ each independently represent H, deuterium, hydroxy, amino, mercapto, nitro, halogen, cyano, halogenated C₁₋₆ alkyl, deuterated C₁₋₆ alkyl, C₁₋₆ alkoxy, halogenated C₁₋₆ alkoxy, C₃₋₆ cycloalkyl, halogenated C₃₋₆ cycloalkyl, deuterated C₃₋₆ cycloalkyl, 4-to 10-membered heterocyclyl, and any combination thereof, and
n1, n2, n3, n4, m3, m4, and m5 each independently represent an integer of 1, 2, 3, 4, 5, 6, 7, 8, 9, or 10.

16. The compound of Formula (I) or salts, enantiomers, stereoisomers, solvates, isotopically enriched analogs, prodrugs, or polymorphs thereof as claimed in claim 1, wherein R_{w1} represents:
—CH₂—, —(CH₂)₂—, —(CH₂)₃—, —(CH₂)₄—, —(CH₂)₅—, —(CH₂)₆—, —(CH₂)₇—, —(CH₂)₈—, —(CH₂)₉—, —(CH₂)₁₀—, —(CH₂)₁₁—, —(CH₂)₁₂—, —(CH₂)₁₃—, —(CH₂)₁₄—, —(CH₂)₁₅—; —O—CH₂—, —O—(CH₂)₂—, —O—(CH₂)₃—, —O—(CH₂)₄—, —O—(CH₂)₅—, —O—(CH₂)₆—, —O—(CH₂)₇—, —O—(CH₂)₈—, —O—(CH₂)₉—, —O—(CH₂)₁₀—, —(CH₂)₁—O—, —(CH₂)₂—O—, —(CH₂)₃—O—, —(CH₂)₄—O—, —(CH₂)₅—O—, —(CH₂)₆—O—, —(CH₂)₇—O—, —(CH₂)₈—O—, —(CH₂)₉—O—, —(CH₂)₁₀—O—, —CH₂—O—CH₂—, —CH₂—O—(CH₂)₂—, —(CH₂)₁—O—(CH₂)₃—, —(CH₂)₁—O—(CH₂)₄—, —(CH₂)₁—O—(CH₂)₅—, —(CH₂)₁—O—(CH₂)₆—, —(CH₂)₁—O—(CH₂)₇—, —(CH₂)₁—O—(CH₂)₈—, —(CH₂)₁—O—(CH₂)₉—, —(CH₂)₁—O—(CH₂)₁₀—, —(CH₂)₂—O—(CH₂)₁—, —(CH₂)₂—O—(CH₂)₂—, —(CH₂)₂—O—(CH₂)₃—, —(CH₂)₂—O—(CH₂)₄—, —(CH₂)₂—O—(CH₂)₅—, —(CH₂)₂—O—(CH₂)₆—, —(CH₂)₂—O—(CH₂)₇—, —(CH₂)₂—O—(CH₂)₈—, —(CH₂)₂—O—(CH₂)₉—, —(CH₂)₂—O—(CH₂)₁₀—, —(CH₂)₃—O—(CH₂)₁—, —(CH₂)₃—O—(CH₂)₂—, —(CH₂)₃—O—(CH₂)₃—, —(CH₂)₃—O—(CH₂)₄—, —(CH₂)₃—O—(CH₂)₅—, —(CH₂)₃—O—(CH₂)₆—, —(CH₂)₃—O—(CH₂)₇—, —(CH₂)₄—O—(CH₂)₁—, —(CH₂)₄—O—(CH₂)₂—, —(CH₂)₄—O—(CH₂)₃—, —(CH₂)₄—O—(CH₂)₄—, —(CH₂)₄—O—(CH₂)₅—, —(CH₂)₄—O—(CH₂)₆—, —(CH₂)₅—O—(CH₂)₁—, —(CH₂)₅—O—(CH₂)₂—, —(CH₂)₅—O—(CH₂)₃—, —(CH₂)₅—O—(CH₂)₄—, —(CH₂)₅—O—(CH₂)₅—, —(CH₂)₆—O—(CH₂)₁—, —(CH₂)₆—O—(CH₂)₂—, —(CH₂)₆—O—(CH₂)₃—, —(CH₂)₆—O—(CH₂)₄—, —(CH₂)₇—O—(CH₂)₁—, —(CH₂)₇—O—(CH₂)₂—, —(CH₂)₇—O—(CH₂)₃—, —(CH₂)₈—O—(CH₂)₁—, —(CH₂)₈—O—(CH₂)₂—, —CH(CH₃)—O—(CH₂)₁—, —CH(CH₃)—O—(CH₂)₂—, —CH(CH₃)—O—(CH₂)₃—, —CH(CH₃)—O—(CH₂)₄—, —CH(CH₃)—O—(CH₂)₅—, —CH(CH₃)—O—(CH₂)₆—, —CH(CH₃)—O—(CH₂)₇—, —CH(CH₃)—O—(CH₂)₈—, —CH(CH₃)—O—(CH₂)₉—, —CH(CH₃)—O—(CH₂)₁₀—, —(O(CH₂)₂)₂—, —(O(CH₂)₂)₃—, —(O(CH₂)₂)₄—, —(O(CH₂)₂)₅—, —(O(CH₂)₂)₆—, —(O(CH₂)₂)₇—, —CH₂—(O(CH₂)₂)₂—, —CH₂—(O(CH₂)₂)₃—, —CH₂—(O(CH₂)₂)₄—, —CH₂—(O(CH₂)₂)₅—, —CH₂—(O(CH₂)₂)₆—, —CH₂—(O(CH₂)₂)₇—, —CH₂—(O(CH₂)₂)₁—OCH₂—, —CH₂—(O(CH₂)₂)₂—OCH₂—, —CH₂—(O(CH₂)₂)₃—OCH₂—, —CH₂—(O(CH₂)₂)₄—OCH₂—, —CH₂—(O(CH₂)₂)₅—OCH₂—, —(CH₂)₂—(O(CH₂)₂)₂—, —(CH₂)₂—(O(CH₂)₂)₃—, —(CH₂)₂—(O(CH₂)₂)₄—, —(CH₂)₂—(O(CH₂)₂)₅—, —(CH₂)₂—(O(CH₂)₂)₆—, —(CH₂)₃—(O(CH₂)₂)₂—, —(CH₂)₃—(O(CH₂)₂)₃—, —(CH₂)₃—(O(CH₂)₂)₄—, —(CH₂)₃—(O(CH₂)₂)₅—, —(CH₂)₄—(O(CH₂)₂)₂—, —(CH₂)₄—(O(CH₂)₂)₃—, —(CH₂)₄—(O(CH₂)₂)₄—, —(CH₂)₄—(O(CH₂)₂)₅—, —CH₂—(O(CH₂)₃)₂—, —CH₂—(O(CH₂)₃)₃—, —CH₂—(O(CH₂)₃)₄—, —(CH₂)₂—(O(CH₂)₃)₂—, —(CH₂)₂—(O(CH₂)₃)₃—, —(CH₂)₂—(O(CH₂)₃)₄—, —(CH₂)₃—(O(CH₂)₃)₂—, —(CH₂)₃—(O(CH₂)₃)₃—, —(CH₂)₃—(O(CH₂)₃)₄—, —CH₂—O—(CH₂)₂—O—(CH₂)₃—, —CH₂—(O(CH₂)₂)₂—(O(CH₂)₃)₂—, (CH₂)₂—O—(CH₂)₂—O—(CH₂)₃—, —(CH₂)₂—(O(CH₂)₂)₂—(O(CH₂)₃)₂—, —(CH₂)₃—O—(CH₂)₂—O—(CH₂)₃—, —(CH₂)₃—(O(CH₂)₂)₂—(O(CH₂)₃)₂—, —CH₂—O—(CH₂)₃—O—(CH₂)₂—, —CH₂—(O(CH₂)₃)₂—(O(CH₂)₂)₂—, —(CH₂)₂—O—(CH₂)₃—O—(CH₂)₂—, —(CH₂)₂—(O(CH₂)₃)₂—(O(CH₂)₂)₂—, —(CH₂)₃—O—(CH₂)₃—O—(CH₂)₂—, —(CH₂)₃—(O(CH₂)₃)₂—(O(CH₂)₂)₂—, —CH₂—O—(CH₂)₂—O—CH₂—, —(CH₂)₂—O—(CH₂)₂—O—CH₂—, —(CH₂)₂—(O(CH₂)₂)₂—O—(CH₂)₃—, —(CH₂)₂—(O(CH₂)₂)₃—O—(CH₂)₃—, —(CH₂)₂—(O(CH₂)₂)₄—O—(CH₂)₃—, —(CH₂)₅—(O(CH₂)₂)₂—O—(CH₂)₅—, —(CH₂)₅—(O(CH₂)₂)₂—O—(CH₂)₆—, —(CH₂)₁—N(Rₐ₇)—(CH₂)₁—, —(CH₂)₁—N(Rₐ₇)—(CH₂)₂—, —(CH₂)₁—N(Rₐ₇)—(CH₂)₃—, —(CH₂)₁—N(Rₐ₇)—(CH₂)₄—, —(CH₂)₁—N(Rₐ₇)—(CH₂)₅—, —(CH₂)₁—N(Rₐ₇)—(CH₂)₆—, —(CH₂)₁—N(Rₐ₇)—(CH₂)₇—, —(CH₂)₁—N(Rₐ₇)—(CH₂)₈—, —(CH₂)₁—N(Rₐ₇)—(CH₂)₉—, —(CH₂)₁—N(Rₐ₇)—(CH₂)₁₀—, —N(Rₐ₇)—(CH₂)₁—, —N(Rₐ₇)—(CH₂)₂—, —N(Rₐ₇)—(CH₂)₃—, —N(Rₐ₇)—(CH₂)₄—, —N(Rₐ₇)—(CH₂)₅—, —N(Rₐ₇)—(CH₂)₆—, —N(Rₐ₇)—(CH₂)₇—, —N(Rₐ₇)—(CH₂)₈—, —N(Rₐ₇)—(CH₂)₉—, —N(Rₐ₇)—(CH₂)₁₀—, —(CH₂)₂—N(Rₐ₇)—(CH₂)₁—, —(CH₂)₂—N(Rₐ₇)—(CH₂)₂—, —(CH₂)₂—N(Rₐ₇)—(CH₂)₃—, —(CH₂)₂—N(Rₐ₇)—(CH₂)₄—, —(CH₂)₂—N(Rₐ₇)—(CH₂)₅—, —(CH₂)₂—N(Rₐ₇)—(CH₂)₆—, —(CH₂)₂—N(Rₐ₇)—(CH₂)₇—, —(CH₂)₂—N(Rₐ₇)—(CH₂)₈—, —(CH₂)₂—N(Rₐ₇)—(CH₂)₉—, —(CH₂)₂—N(Rₐ₇)—(CH₂)₁₀—, —(CH₂)₃—N(Rₐ₇)—(CH₂)₁—, —(CH₂)₃—N(Rₐ₇)—(CH₂)₂—, —(CH₂)₃—N(Rₐ₇)—(CH₂)₃—, —(CH₂)₄—N(Rₐ₇)—(CH₂)₁—, —(CH₂)₄—N(Rₐ₇)—(CH₂)₂—, —(CH₂)₄—N(Rₐ₇)—(CH₂)₃—, —(CH₂)₄—N(Rₐ₇)—(CH₂)₄—, —(CH₂)₅—N(Rₐ₇)—(CH₂)₁—, —(CH₂)₅—N(Rₐ₇)—(CH₂)₂—, —(CH₂)₅—N(Rₐ₇)—(CH₂)₃—, —(CH₂)₅—N(Rₐ₇)—(CH₂)₄—, —(CH₂)₅—N(Rₐ₇)—(CH₂)₅—, —(CH₂)₆—N(Rₐ₇)—(CH₂)₁—, —(CH₂)₆—N(Rₐ₇)—(CH₂)₂—, —(CH₂)₆—N(Rₐ₇)—(CH₂)₃—, —(CH₂)₇—N(Rₐ₇)—(CH₂)₁—, —(CH₂)₇—N(Rₐ₇)—(CH₂)₂—, —(CH₂)₇—N(Rₐ₇)—(CH₂)₃—, —(CH₂)₈—N(Rₐ₇)—(CH₂)₁—, —(CH₂)₈—N(Rₐ₇)—(CH₂)₂—, —(CH₂)₈—N(Rₐ₇)—(CH₂)₃—, —CH(CH₃)—N(Rₐ₇)—(CH₂)₁—, —CH(CH₃)—N(Rₐ₇)—(CH₂)₂—, —CH(CH₃)—N(Rₐ₇)—(CH₂)₃—, —CH(CH₃)—N(Rₐ₇)—(CH₂)₄—, —CH(CH₃)—N(Rₐ₇)—(CH₂)₅—, —CH(CH₃)—N(Rₐ₇)—(CH₂)₆—, —CH(CH₃)—N(Rₐ₇)—(CH₂)₇—, —CH(CH₃)—N(Rₐ₇)—(CH₂)₈—, —CH(CH₃)—N(Rₐ₇)—(CH₂)₉—, —CH(CH₃)—N(Rₐ₇)—(CH₂)₁₀—, —C(O)NHCH₂—, —C(O)NH(CH₂)₂—, —C(O)NH(CH₂)₃—, —C(O)NH(CH₂)₄—, —C(O)NH(CH₂)₅—, —CH₂C(O)NHCH₂—, —(CH₂)₂C(O)NH(CH₂)₂—, —(CH₂)₂C(O)NH(CH₂)₃—, —(CH₂)₂C(O)NH(CH₂)₄—, —(CH₂)₂C(O)NH(CH₂)₅—, —(CH₂)₃C(O)NH(CH₂)₃—, —(CH₂)₃C(O)NH(CH₂)₄—, —(CH₂)₄C(O)NH(CH₂)₄—, —(CH₂)₅C(O)NH(CH₂)₅—, —(CH₂)₆C(O)NH(CH₂)₇—, —(CH₂)₆C(O)NH(CH₂)₆—, —(CH₂)₇C(O)NH(CH₂)₇—, —(CH₂)₂C(O)NH(CH₂)₂—O—(CH₂)₂—, —NHC(O)CH₂—, —NHC(O)(CH₂)₂—, —NHC(O)(CH₂)₃—, —NHC(O)(CH₂)₄—, —NHC(O)(CH₂)₅—, —CH₂NHC(O)CH₂—, —(CH₂)₂NHC(O)(CH₂)₂—, —(CH₂)₂NHC(O)(CH₂)₃—, —(CH₂)₂NHC(O)(CH₂)₄—, —(CH₂)₂NHC(O)(CH₂)₅—, —(CH₂)₃NHC(O)(CH₂)₃—, —(CH₂)₃NHC(O)(CH₂)₄—, —(CH₂)₄NHC(O)(CH₂)₄—, —(CH₂)₅NHC(O)(CH₂)₅—, —(CH₂)₆NHC(O)(CH₂)₇—, —(CH₂)₆NHC(O)(CH₂)₆—, —(CH₂)₇NHC(O)(CH₂)₇—, —(CH₂)₄NHC(O)(CH₂)₈—, —(CH₂)₂NHC(O)(CH₂)₂—O—(CH₂)₂—, —(CH₂)₄NHC(O)CH₂—,-phenylene-CH₂—,-phenylene-(CH₂)₂—,-phenylene-(CH₂)₃—,-phenylene-(CH₂)₄—,-phenylene-(CH₂)₅—,-phenylene-(CH₂)₆—,-phenylene-(CH₂)₇—,-phenylene-(CH₂)₈—, —CH₂-phenylene-CH₂—, —CH₂-phenylene-(CH₂)₂—, —CH₂-phenylene-(CH₂)₃—, —CH₂-phenylene-(CH₂)₄—, —CH₂-phenylene-(CH₂)₅—, —CH₂-phenylene-(CH₂)₆—, —CH₂-phenylene-(CH₂)₇—, —CH₂-phenylene-(CH₂)₈—, —(CH₂)₂-phenylene-(CH₂)₁—, —(CH₂)₂-phenylene-(CH₂)₂—, —(CH₂)₂-phenylene-(CH₂)₃—, —(CH₂)₂-phenylene-(CH₂)₄—, —(CH₂)₂-phenylene-(CH₂)₅—, —(CH₂)₂-phenylene-(CH₂)₆—, —(CH₂)₂-phenylene-(CH₂)₇—, —(CH₂)₂-phenylene-(CH₂)₈—, —(CH₂)₃-phenylene-CH₂—, —(CH₂)₃-phenylene-(CH₂)₂—, —(CH₂)₃-phenylene-(CH₂)₃—, —(CH₂)₃-phenylene-(CH₂)₄—, —(CH₂)₃-phenylene-(CH₂)₅—, —(CH₂)₃-phenylene-(CH₂)₆—, —(CH₂)₃-phenylene-(CH₂)₇—, —(CH₂)₃-phenylene-(CH₂)₈—, —(CH₂)₄-phenylene-CH₂—, —(CH₂)₄-phenylene-(CH₂)₂—, —(CH₂)₄-phenylene-(CH₂)₃—, —(CH₂)₄-phenylene-(CH₂)₄—, —(CH₂)₄-phenylene-(CH₂)₅—, —(CH₂)₄-phenylene-(CH₂)₆—, —(CH₂)₄-phenylene-(CH₂)₇—, —(CH₂)₄-phenylene-(CH₂)₈—, —(CH₂)₅-phenylene-(CH₂)₁—, —(CH₂)₅-phenylene-(CH₂)₂—, —(CH₂)₅-phenylene-(CH₂)₃—, —(CH₂)₅-phenylene-(CH₂)₄—, —(CH₂)₅-phenylene-(CH₂)₅—, —(CH₂)₅-phenylene-(CH₂)₆—, —(CH₂)₅-phenylene-(CH₂)₇—, —(CH₂)₅-phenylene-(CH₂)₈—, —(CH₂)₆-phenylene-(CH₂)₁—, —(CH₂)₆-phenylene-(CH₂)₂—, —(CH₂)₆-phenylene-(CH₂)₃—, —(CH₂)₆-phenylene-(CH₂)₄—, —(CH₂)₆-phenylene-(CH₂)₅—, —(CH₂)₆-phenylene-(CH₂)₆—, —(CH₂)₆-phenylene-(CH₂)₇—, —(CH₂)₆-phenylene-(CH₂)₈—, —(CH₂)₇-phenylene-(CH₂)₁—, —(CH₂)₇-phenylene-(CH₂)₂—, —(CH₂)₇-phenylene-(CH₂)₃—, —(CH₂)₇-phenylene-(CH₂)₄—, —(CH₂)₇-phenylene-(CH₂)₈—, —(CH₂)₈-phenylene-CH₂—, —(CH₂)₈-phenylene-(CH₂)₂—, —(CH₂)₈-phenylene-(CH₂)₃—, —(CH₂)₈-phenylene-(CH₂)₄—, —(CH₂)₈-phenylene-(CH₂)₅—, —(CH₂)₈-phenylene-(CH₂)₆—, —(CH₂)₈-phenylene-(CH₂)₇—, —N(Rₐ₇)—CH₂-phenylene-CH₂—, —N(Rₐ₇)—CH₂-phenylene-(CH₂)₂—, —N(Rₐ₇)—CH₂-phenylene-(CH₂)₃—, —N(Rₐ₇)—CH₂-phenylene-(CH₂)₄—, —N(Rₐ₇)—CH₂-phenylene-(CH₂)₅—, —N(Rₐ₇)—CH₂-phenylene-(CH₂)₆—, —N(Rₐ₇)—CH₂-phenylene-(CH₂)₇—, —N(Rₐ₇)—CH₂-phenylene-(CH₂)₈—, —CH₂—N(Rₐ₇)—CH₂-phenylene-CH₂—, —CH₂—N(Rₐ₇)—CH₂-phenylene-(CH₂)₂—, —CH₂—N(Rₐ₇)—CH₂-phenylene-(CH₂)₃—, —CH₂—N(Rₐ₇)—CH₂-phenylene-(CH₂)₄—, —CH₂—N(Rₐ₇)—CH₂-phenylene-(CH₂)₅—, —CH₂—N(Rₐ₇)—CH₂-phenylene-(CH₂)₆—, —CH₂—N(Rₐ₇)—CH₂-phenylene-(CH₂)₇—, —CH₂—N(Rₐ₇)—CH₂-phenylene-(CH₂)₈—, —CH₂—N(Rₐ₇)—(CH₂)₂-phenylene-CH₂—, —CH₂—N(Rₐ₇)—(CH₂)₂-phenylene-(CH₂)₂—, —CH₂—N(Rₐ₇)—(CH₂)₂-phenylene-(CH₂)₃—, —CH₂—N(Rₐ₇)—(CH₂)₂-phenylene-(CH₂)₄—, —CH₂—N(Rₐ₇)—(CH₂)₂-phenylene-(CH₂)₅—, —CH₂—N(Rₐ₇)—(CH₂)₂-phenylene-(CH₂)₆—, —CH₂—N(Rₐ₇)—(CH₂)₂-phenylene-(CH₂)₇—, —CH₂—N(Rₐ₇)—(CH₂)₂-phenylene-(CH₂)₈—, —(CH₂)₂—N(Rₐ₇)—CH₂-phenylene-CH₂—, —(CH₂)₂—N(Rₐ₇)—CH₂-phenylene-(CH₂)₂—, —(CH₂)₂—N(Rₐ₇)—CH₂-phenylene-(CH₂)₃—, —(CH₂)₂—N(Rₐ₇)—CH₂-phenylene-(CH₂)₄—, —(CH₂)₂—N(Rₐ₇)—CH₂-phenylene-(CH₂)₅—, —(CH₂)₂—N(Rₐ₇)—CH₂-phenylene-(CH₂)₆—, —(CH₂)₂—N(Rₐ₇)—CH₂-phenylene-(CH₂)₇—, —(CH₂)₂—N(Rₐ₇)—CH₂-phenylene-(CH₂)₈—, —(CH₂)₃—N(Rₐ₇)—CH₂-phenylene-CH₂—, —(CH₂)₃—N(Rₐ₇)—CH₂-phenylene-(CH₂)₂—, —(CH₂)₃—N(Rₐ₇)—CH₂-phenylene-(CH₂)₃—, —(CH₂)₃—N(Rₐ₇)—CH₂-phenylene-(CH₂)₈—, —(CH₂)₄—N(Rₐ₇)—CH₂-phenylene-CH₂—, —(CH₂)₄—N(Rₐ₇)—CH₂-phenylene-(CH₂)₂—, —(CH₂)₄—N(Rₐ₇)—CH₂-phenylene-(CH₂)₃—, —(CH₂)₄—N(Rₐ₇)—CH₂-phenylene-(CH₂)₈—, —(CH₂)₅—N(Rₐ₇)—CH₂-phenylene-(CH₂)₃—, —(CH₂)₅—N(Rₐ₇)—CH₂-phenylene-(CH₂)₈—, —(CH₂)₆—N(Rₐ₇)—CH₂-phenylene-(CH₂)₃—, —(CH₂)₆—N(Rₐ₇)—CH₂-phenylene-(CH₂)₈—, —(CH₂)₇—N(Rₐ₇)—CH₂-phenylene-(CH₂)₃—, —(CH₂)₈—N(Rₐ₇)—CH₂-phenylene-CH₂—, —(CH₂)₈—N(Rₐ₇)—CH₂-phenylene-(CH₂)₂—, —(CH₂)₈—N(Rₐ₇)—CH₂-phenylene-(CH₂)₃—, —(CH₂)₈—N(Rₐ₇)—CH₂-phenylene-(CH₂)₄—, (CH₂)₈—N(Rₐ₇)—CH₂-phenylene-(CH₂)₅—, —(CH₂)₈—N(Rₐ₇)—CH₂-phenylene-(CH₂)₆—,-piperidinylene-CH₂—,-piperidinylene- (CH₂)₂—,-piperidinylene-(CH₂)₃—,-piperidinylene-(CH₂)₄—,-piperidinylene-(CH₂)₅—,-piperidinylene-(CH₂)₆—,-piperidinylene-(CH₂)₇—,-piperidinylene-(CH₂)₈—, —CH₂-piperidinylene-CH₂—, —CH₂-piperidinylene-(CH₂)₂—, —CH₂-piperidinylene-(CH₂)₃—, —CH₂-piperidinylene-(CH₂)₄—, —CH₂-piperidinylene-(CH₂)₅—, —CH₂-piperidinylene-(CH₂)₆—, —CH₂-piperidinylene-(CH₂)₇—, —CH₂-piperidinylene-(CH₂)₈—, —(CH₂)₂-piperidinylene-(CH₂)₁—, —(CH₂)₂-piperidinylene-(CH₂)₂—, —(CH₂)₂-piperidinylene-(CH₂)₃—, —(CH₂)₂-piperidinylene-(CH₂)₄—, —(CH₂)₂-piperidinylene-(CH₂)₅—, —(CH₂)₂-piperidinylene-(CH₂)₆—, —(CH₂)₂-piperidinylene-(CH₂)₇—, —(CH₂)₂-piperidinylene-(CH₂)₈—, —(CH₂)₃-piperidinylene-CH₂—, —(CH₂)₃-piperidinylene-(CH₂)₂—, —(CH₂)₃-piperidinylene-(CH₂)₃—, —(CH₂)₃-piperidinylene-(CH₂)₄—, —(CH₂)₃-piperidinylene-(CH₂)₅—, —(CH₂)₃-piperidinylene-(CH₂)₆—, —(CH₂)₃-piperidinylene-(CH₂)₇—, —(CH₂)₃-piperidinylene-(CH₂)₈—, —(CH₂)₄-piperidinylene-CH₂—, —(CH₂)₄-piperidinylene-(CH₂)₂—, —(CH₂)₄-piperidinylene-(CH₂)₃—, —(CH₂)₄-piperidinylene-(CH₂)₄—, —(CH₂)₄-piperidinylene-(CH₂)₅—, —(CH₂)₄-piperidinylene-(CH₂)₆—, —(CH₂)₄-piperidinylene-(CH₂)₇—, —(CH₂)₄-piperidinylene-(CH₂)₈—, —(CH₂)₅-piperidinylene-(CH₂)₁—, —(CH₂)₅-piperidinylene-(CH₂)₂—, —(CH₂)₅-piperidinylene-(CH₂)₃—, —(CH₂)₅-piperidinylene-(CH₂)₄—, —(CH₂)₅-piperidinylene-(CH₂)₅—, —(CH₂)₅-piperidinylene-(CH₂)₆—, —(CH₂)₅-piperidinylene-(CH₂)₇—, —(CH₂)₅-piperidinylene-(CH₂)₈—, —(CH₂)₆-piperidinylene-(CH₂)₁—, —(CH₂)₆-piperidinylene-(CH₂)₂—, —(CH₂)₆-piperidinylene-(CH₂)₃—, —(CH₂)₆-piperidinylene-(CH₂)₄—, —(CH₂)₆-piperidinylene-(CH₂)₅—, —(CH₂)₆-piperidinylene-(CH₂)₆—, —(CH₂)₆-piperidinylene-(CH₂)₇—, —(CH₂)₆-piperidinylene-(CH₂)₈—, —(CH₂)₇-piperidinylene-(CH₂)₁—, —(CH₂)₇-piperidinylene-(CH₂)₂—, —(CH₂)₇-piperidinylene-(CH₂)₃—, —(CH₂)₇-piperidinylene-(CH₂)₄—, —(CH₂)₇-piperidinylene-(CH₂)₈—, —(CH₂)₈-piperidinylene-CH₂—, —(CH₂)₈-piperidinylene-(CH₂)₂—, —(CH₂)₈-piperidinylene-(CH₂)₃—, —(CH₂)₈-piperidinylene-(CH₂)₄—, —(CH₂)₈-piperidinylene-(CH₂)₅—, —(CH₂)₈-piperidinylene-(CH₂)₆—, —(CH₂)₈-piperidinylene-(CH₂)₇—, —N(Rₐ₇)—CH₂-piperidinylene-CH₂—, —N(Rₐ₇)—CH₂-piperidinylene-(CH₂)₂—, —N(Rₐ₇)—CH₂-piperidinylene-(CH₂)₃—, —N(Rₐ₇)—CH₂-piperidinylene-(CH₂)₄—, —N(Rₐ₇)—CH₂-piperidinylene-(CH₂)₅—, —N(Rₐ₇)—CH₂-piperidinylene-(CH₂)₆—, —N(Rₐ₇)—CH₂-piperidinylene-(CH₂)₇—, —N(Rₐ₇)—CH₂-piperidinylene-(CH₂)₈—, —CH₂—N(Rₐ₇)—CH₂-piperidinylene-CH₂—, —CH₂—N(Rₐ₇)—CH₂-piperidinylene-(CH₂)₂—, —CH₂—N(Rₐ₇)—CH₂-piperidinylene-(CH₂)₃—, —CH₂—N(Rₐ₇)—CH₂-piperidinylene-(CH₂)₄—, —CH₂—N(Rₐ₇)—CH₂-piperidinylene-(CH₂)₅—, —CH₂—N(Rₐ₇)—CH₂-piperidinylene-(CH₂)₆—, —CH₂—N(Rₐ₇)—CH₂-piperidinylene-(CH₂)₇—, —CH₂—N(Rₐ₇)—CH₂-piperidinylene-(CH₂)₈—, —CH₂—N(Rₐ₇)—(CH₂)₂-piperidinylene-CH₂—, —CH₂—N(Rₐ₇)—(CH₂)₂-piperidinylene-(CH₂)₂—, —CH₂—N(Rₐ₇)—(CH₂)₂-piperidinylene-(CH₂)₃—, —CH₂—N(Rₐ₇)—(CH₂)₂-piperidinylene-(CH₂)₄—, —CH₂—N(Rₐ₇)—(CH₂)₂-piperidinylene-(CH₂)₅—, —CH₂—N(Rₐ₇)—(CH₂)₂-piperidinylene-(CH₂)₆—, —CH₂—N(Rₐ₇)—(CH₂)₂-piperidinylene-(CH₂)₇—, —CH₂—N(Rₐ₇)—(CH₂)₂-piperidinylene-(CH₂)₈—, —(CH₂)₂—N(Rₐ₇)—CH₂-piperidinylene-CH₂—, —(CH₂)₂—N(Rₐ₇)—CH₂-piperidinylene-(CH₂)₂—, —(CH₂)₂—N(Rₐ₇)—CH₂-piperidinylene-(CH₂)₃—, —(CH₂)₂—N(Rₐ₇)—CH₂-piperidinylene-(CH₂)₄—, (CH₂)₂—N(Rₐ₇)—CH₂-piperidinylene-(CH₂)₅—, —(CH₂)₂—N(Rₐ₇)—CH₂-piperidinylene-(CH₂)₆—, —(CH₂)₂—N(Rₐ₇)—CH₂-piperidinylene-(CH₂)₇—, —(CH₂)₂—N(Rₐ₇)—CH₂-piperidinylene-(CH₂)₈—, —(CH₂)₃—N(Rₐ₇)—CH₂-piperidinylene-CH₂—, —(CH₂)₃—N(Rₐ₇)—CH₂-piperidinylene-(CH₂)₂—, —(CH₂)₃—N(Rₐ₇)—CH₂-piperidinylene-(CH₂)₃—, —(CH₂)₃—N(Rₐ₇)—CH₂-piperidinylene-(CH₂)₈—, —(CH₂)₄—N(Rₐ₇)—CH₂-piperidinylene-CH₂—, —(CH₂)₄—N(Rₐ₇)—CH₂-piperidinylene-(CH₂)₂—, —(CH₂)₄—N(Rₐ₇)—CH₂-piperidinylene-(CH₂)₃—, —(CH₂)₄—N(Rₐ₇)—CH₂-piperidinylene-(CH₂)₈—, —(CH₂)₅—N(Rₐ₇)—CH₂-piperidinylene-(CH₂)₃—, —(CH₂)₅—N(Rₐ₇)—CH₂-piperidinylene-(CH₂)₈—, —(CH₂)₆—N(Rₐ₇)—CH₂-piperidinylene-(CH₂)₃—, (CH₂)₆—N(Rₐ₇)—CH₂-piperidinylene-(CH₂)₈—, —(CH₂)₇—N(Rₐ₇)—CH₂-piperidinylene-(CH₂)₃—, —(CH₂)₈—N(Rₐ₇)—CH₂-piperidinylene-CH₂—, —(CH₂)₈—N(Rₐ₇)—CH₂-piperidinylene-(CH₂)₂—, —(CH₂)₈—N(Rₐ₇)—CH₂-piperidinylene-(CH₂)₃—, —(CH₂)₈—N(Rₐ₇)—CH₂-piperidinylene-(CH₂)₄—, —(CH₂)₈—N(Rₐ₇)—CH₂-piperidinylene-(CH₂)₅—, —(CH₂)₈—N(Rₐ₇)—CH₂-piperidinylene-(CH₂)₆—,-piperazinylene-CH₂—,-piperazinylene-(CH₂)₂—,-piperazinylene-(CH₂)₃—,-piperazinylene-(CH₂)₄—,-piperazinylene-(CH₂)₅—,-piperazinylene-(CH₂)₆—,-piperazinylene-(CH₂)₇—,-piperazinylene-(CH₂)₈—, —CH₂-piperazinylene-CH₂—, —CH₂-piperazinylene-(CH₂)₂—, —CH₂-piperazinylene-(CH₂)₃—, —CH₂-piperazinylene-(CH₂)₄—, —CH₂-piperazinylene-(CH₂)₅—, —CH₂-piperazinylene-(CH₂)₆—, —CH₂-piperazinylene-(CH₂)₇—, —CH₂-piperazinylene-(CH₂)₈—, —(CH₂)₂-piperazinylene-(CH₂)₁—, —(CH₂)₂-piperazinylene-(CH₂)₂—, —(CH₂)₂-piperazinylene-(CH₂)₃—, —(CH₂)₂-piperazinylene-(CH₂)₄—, —(CH₂)₂-piperazinylene-(CH₂)₅—, —(CH₂)₂-piperazinylene-(CH₂)₆—, —(CH₂)₂-piperazinylene-(CH₂)₇—, —(CH₂)₂-piperazinylene-(CH₂)₈—, —(CH₂)₃-piperazinylene-CH₂—, —(CH₂)₃-piperazinylene-(CH₂)₂—, —(CH₂)₃-piperazinylene-(CH₂)₃—, —(CH₂)₃-piperazinylene-(CH₂)₄—, —(CH₂)₃-piperazinylene-(CH₂)₅—, —(CH₂)₃-piperazinylene-(CH₂)₆—, —(CH₂)₃-piperazinylene-(CH₂)₇—, —(CH₂)₃-piperazinylene-(CH₂)₈—, —(CH₂)₄-piperazinylene-CH₂—, —(CH₂)₄-piperazinylene-(CH₂)₂—, —(CH₂)₄-piperazinylene-(CH₂)₃—, —(CH₂)₄-piperazinylene-(CH₂)₄—, —(CH₂)₄-piperazinylene-(CH₂)₅—, —(CH₂)₄-piperazinylene-(CH₂)₆—, —(CH₂)₄-piperazinylene-(CH₂)₇—, —(CH₂)₄-piperazinylene-(CH₂)₈—, —(CH₂)₅-piperazinylene-(CH₂)₁—, —(CH₂)₅-piperazinylene-(CH₂)₂—, —(CH₂)₅-piperazinylene-(CH₂)₃—, —(CH₂)₅-piperazinylene-(CH₂)₄—, —(CH₂)₅-piperazinylene-(CH₂)₅—, —(CH₂)₅-piperazinylene-(CH₂)₆—, —(CH₂)₅-piperazinylene-(CH₂)₇—, —(CH₂)₅-piperazinylene-(CH₂)₈—, —(CH₂)₆-piperazinylene-(CH₂)₁—, —(CH₂)₆-piperazinylene-(CH₂)₂—, —(CH₂)₆-piperazinylene-(CH₂)₃—, —(CH₂)₆-piperazinylene-(CH₂)₄—, —(CH₂)₆-piperazinylene-(CH₂)₅—, —(CH₂)₆-piperazinylene-(CH₂)₆—, —(CH₂)₆-piperazinylene-(CH₂)₇—, —(CH₂)₆-piperazinylene-(CH₂)₈—, —(CH₂)₇-piperazinylene-(CH₂)₁—, —(CH₂)₇-piperazinylene-(CH₂)₂—, —(CH₂)₇-piperazinylene-(CH₂)₃—, —(CH₂)₇-piperazinylene-(CH₂)₄—, —(CH₂)₈-piperazinylene-CH₂—, —(CH₂)₈-piperazinylene-(CH₂)₂—, —(CH₂)₈—piperazinylene-(CH₂)₃—, —(CH₂)₈-piperazinylene-(CH₂)₄—, —(CH₂)₈-piperazinylene-(CH₂)₅—, or —(CH₂)₈-piperazinylene-(CH₂)₆—;
wherein each Rₐ₇ independently represents H or C₁₋₃ alkyl;
the phenylene, piperazinylene and piperidinylene are each independently optionally substituted with a substituent selected from the group consisting of deuterium, hydroxy, amino, mercapto, nitro, halogen, cyano, oxo, C₁₋₆ alkyl, halogenated C₁₋₆ alkyl, deuterated C₁₋₆ alkyl, C₁₋₆ alkoxy, halogenated C₁₋₆ alkoxy, C₃₋₆ cycloalkyl, halogenated C₃₋₆ cycloalkyl, deuterated C₃₋₆ cycloalkyl, 4-to 10-membered heterocyclyl, and any combination thereof.

17. The compound of Formula (I) or salts, enantiomers, stereoisomers, solvates, isotopically enriched analogs, prodrugs, or polymorphs thereof as claimed in claim 1, wherein LIN represents: a bond, C(O), C(O)NH, NHC(O), —CH₂—, —(CH₂)₂—, —(CH₂)₃—, —(CH₂)₄—, —(CH₂)₅—, —(CH₂)₆—, —(CH₂)₇—, —(CH₂)₈—, —(CH₂)₉—, —(CH₂)₁₀—, —(CH₂)₁₁—, —(CH₂)₁₂—, —(CH₂)₁₃—, —(CH₂)₁₄—, —(CH₂)₁₅—; —O—CH₂—, —O—(CH₂)₂—, —O—(CH₂)₃—, —O—(CH₂)₄—, —O—(CH₂)₅—, —O—(CH₂)₆—, —O—(CH₂)₇—, —O—(CH₂)₈—, —O—(CH₂)₉—, —O—(CH₂)₁₀—, —(CH₂)₁—O—, —(CH₂)₂—O—, —(CH₂)₃—O—, —(CH₂)₄—O—, —(CH₂)₅—O—, —(CH₂)₆—O—, —(CH₂)₇—O—, —(CH₂)₈—O—, —(CH₂)₉—O—, —(CH₂)₁₀—O—, —CH₂—O—CH₂—, —CH₂—O—(CH₂)₂—, —(CH₂)₁—O—(CH₂)₃—, —(CH₂)₁—O—(CH₂)₄—, —(CH₂)₁—O—(CH₂)₅—, —(CH₂)₁—O—(CH₂)₆—, —(CH₂)₁—O—(CH₂)₇—, —(CH₂)₁—O—(CH₂)₈—, —(CH₂)₂—O—(CH₂)₁—, —(CH₂)₂—O—(CH₂)₂—, —(CH₂)₂—O—(CH₂)₃—, —(CH₂)₂—O—(CH₂)₄—, —(CH₂)₂—O—(CH₂)₅—, —(CH₂)₂—O—(CH₂)₆—, —(CH₂)₂—O—(CH₂)₇—, —(CH₂)₂—O—(CH₂)₈—, —(CH₂)₃—O—(CH₂)₁—, —(CH₂)₃—O—(CH₂)₂—, —(CH₂)₃—O—(CH₂)₃—, —(CH₂)₃—O—(CH₂)₄—, —(CH₂)₃—O—(CH₂)₅—, —(CH₂)₃—O—(CH₂)₆—, —(CH₂)₃—O—(CH₂)₇—, —(CH₂)₄—O—(CH₂)₁—, —(CH₂)₄—O—(CH₂)₂—, —(CH₂)₄—O—(CH₂)₃—, —(CH₂)₄—O—(CH₂)₄—, —(CH₂)₄—O—(CH₂)₅—, —(CH₂)₄—O—(CH₂)₆—, —(CH₂)₅—O—(CH₂)₁—, —(CH₂)₅—O—(CH₂)₂—, —(CH₂)₅—O—(CH₂)₃—, —(CH₂)₅—O—(CH₂)₄—, —(CH₂)₅—O—(CH₂)₅—, —(CH₂)₆—O—(CH₂)₁—, —(CH₂)₆—O—(CH₂)₂—, —(CH₂)₆—O—(CH₂)₃—, —(CH₂)₆—O—(CH₂)₄—, —(CH₂)₇—O—(CH₂)₁—, —(CH₂)₇—O—(CH₂)₂—, —(CH₂)₇—O—(CH₂)₃—, —(CH₂)₈—O—(CH₂)₁—, —(CH₂)₈—O—(CH₂)₂—, —CH(CH₃)—O—(CH₂)₁—, —CH(CH₃)—O—(CH₂)₂—, —CH(CH₃)—O—(CH₂)₃—, —CH(CH₃)—O—(CH₂)₄—, —CH(CH₃)—O—(CH₂)₅—, —CH(CH₃)—O—(CH₂)₆—, —CH(CH₃)—O—(CH₂)₇—, —CH(CH₃)—O—(CH₂)₈—, —(O(CH₂)₂)₂—, —(O(CH₂)₂)₃—, —(O(CH₂)₂)₄—, —(O(CH₂)₂)₅—, —(O(CH₂)₂)₆—, —CH₂—(O(CH₂)₂)₂—, —CH₂—(O(CH₂)₂)₃—, —CH₂—(O(CH₂)₂)₄—, —CH₂—(O(CH₂)₂)₅—, —CH₂—(O(CH₂)₂)₆—, —CH₂—(O(CH₂)₂)₁—OCH₂—, —CH₂—(O(CH₂)₂)₂—OCH₂—, —CH₂—(O(CH₂)₂)₃—OCH₂—, —CH₂—(O(CH₂)₂)₄—OCH₂—, —CH₂—(O(CH₂)₂)₅—OCH₂—, —CH₂—(O(CH₂)₂)₆—OCH₂—, —(CH₂)₂—(O(CH₂)₂)₂—, —(CH₂)₂—(O(CH₂)₂)₃—, —(CH₂)₂—(O(CH₂)₂)₄—, —(CH₂)₂—(O(CH₂)₂)₅—, —(CH₂)₂—(O(CH₂)₂)₆—, —(CH₂)₃—(O(CH₂)₂)₂—, —(CH₂)₃—(O(CH₂)₂)₃—, —(CH₂)₃—(O(CH₂)₂)₄—, —(CH₂)₃—(O(CH₂)₂)₅—, —(CH₂)₄—(O(CH₂)₂)₂—, —(CH₂)₄—(O(CH₂)₂)₃—, —(CH₂)₄—(O(CH₂)₂)₄—, —(CH₂)₄—(O(CH₂)₂)₅—, —CH₂—(O(CH₂)₃)₂—, —CH₂—(O(CH₂)₃)₃—, —CH₂—(O(CH₂)₃)₄—, —(CH₂)₂—(O(CH₂)₃)₂—, —(CH₂)₂—(O(CH₂)₃)₃—, —(CH₂)₂—(O(CH₂)₃)₄—, —(CH₂)₃—(O(CH₂)₃)₂—, —(CH₂)₃—(O(CH₂)₃)₃—, —CH₂—O—(CH₂)₂—O—(CH₂)₃—, —CH₂—(O(CH₂)₂)₂—(O(CH₂)₃)₂—, (CH₂)₂—O—(CH₂)₂—O—(CH₂)₃—, —(CH₂)₂—(O(CH₂)₂)₂—(O(CH₂)₃)₂—, —(CH₂)₃—O—(CH₂)₂—O—(CH₂)₃—, —(CH₂)₃—(O(CH₂)₂)₂—(O(CH₂)₃)₂—, —CH₂—O—(CH₂)₃—O—(CH₂)₂—, —CH₂—(O(CH₂)₃)₂—(O(CH₂)₂)₂—, (CH₂)₁—N(Rₐ₇)—(CH₂)₁—, —(CH₂)₁—N(Rₐ₇)—(CH₂)₂—, —(CH₂)₁—N(Rₐ₇)—(CH₂)₃—, —(CH₂)₁—N(Rₐ₇)—(CH₂)₄—, —(CH₂)₁—N(Rₐ₇)—(CH₂)₅—, —(CH₂)₁—N(Rₐ₇)—(CH₂)₆—, —N(Rₐ₇)—(CH₂)₁—, —N(Rₐ₇)—(CH₂)₂—, —N(Rₐ₇)—(CH₂)₃—, —N(Rₐ₇)—(CH₂)₄—, —N(Rₐ₇)—(CH₂)₅—, —N(Rₐ₇)—(CH₂)₆—, —(CH₂)₂—N(Rₐ₇)—(CH₂)₁—, —(CH₂)₂—N(Rₐ₇)—(CH₂)₂—, —(CH₂)₂—N(Rₐ₇)—(CH₂)₃—, —(CH₂)₂—N(Rₐ₇)—(CH₂)₄—, —(CH₂)₂—N(Rₐ₇)—(CH₂)₅—, —(CH₂)₂—N(Rₐ₇)—(CH₂)₆—, —(CH₂)₃—N(Rₐ₇)—(CH₂)₁—, —(CH₂)₃—N(Rₐ₇)—(CH₂)₂—, —(CH₂)₃—N(Rₐ₇)—(CH₂)₃—, —(CH₂)₄—N(Rₐ₇)—(CH₂)₁—, —(CH₂)₄—N(Rₐ₇)—(CH₂)₂—, —(CH₂)₄—N(Rₐ₇)—(CH₂)₃—, —(CH₂)₄—N(Rₐ₇)—(CH₂)₄—, —(CH₂)₅—N(Rₐ₇)—(CH₂)₁—, —(CH₂)₅—N(Rₐ₇)—(CH₂)₂—, —(CH₂)₅—N(Rₐ₇)—(CH₂)₃—, —(CH₂)₅—N(Rₐ₇)—(CH₂)₄—, —(CH₂)₅—N(Rₐ₇)—(CH₂)₅—, —(CH₂)₆—N(Rₐ₇)—(CH₂)₁—, —(CH₂)₆—N(Rₐ₇)—(CH₂)₂—, —(CH₂)₆—N(Rₐ₇)—(CH₂)₃—, —(CH₂)₇—N(Rₐ₇)—(CH₂)₁—, —(CH₂)₇—N(Rₐ₇)—(CH₂)₂—, —(CH₂)₇—N(Rₐ₇)—(CH₂)₃—, —(CH₂)₈—N(Rₐ₇)—(CH₂)₁—, —(CH₂)₈—N(Rₐ₇)—(CH₂)₂—, —(CH₂)₈—N(Rₐ₇)—(CH₂)₃—, —CH(CH₃)—N(Rₐ₇)—(CH₂)₁—, —CH(CH₃)—N(Rₐ₇)—(CH₂)₂—, —CH(CH₃)—N(Rₐ₇)—(CH₂)₃—, —CH(CH₃)—N(Rₐ₇)—(CH₂)₄—, —CH(CH₃)—N(Rₐ₇)—(CH₂)₅—, —CH(CH₃)—N(Rₐ₇)—(CH₂)₆—, —C(O)NHCH₂—, —C(O)NH(CH₂)₂—, —C(O)NH(CH₂)₃—, —C(O)NH(CH₂)₄—, —C(O)NH(CH₂)₅—, —CH₂C(O)NHCH₂—, —(CH₂)₂C(O)NH(CH₂)₂—, —(CH₂)₂C(O)NH(CH₂)₃—, —(CH₂)₂C(O)NH(CH₂)₄—, —(CH₂)₂C(O)NH(CH₂)₅—, —(CH₂)₃C(O)NH(CH₂)₃—, —(CH₂)₃C(O)NH(CH₂)₄—, —(CH₂)₄C(O)NH(CH₂)₄—, —(CH₂)₅C(O)NH(CH₂)₅—, —(CH₂)₂C(O)NH(CH₂)₂—O—(CH₂)₂—, —NHC(O)CH₂—, —NHC(O)(CH₂)₂—, —NHC(O)(CH₂)₃—, —NHC(O)(CH₂)₄—, —NHC(O)(CH₂)₅—, —CH₂NHC(O)CH₂—, —(CH₂)₂NHC(O)(CH₂)₂—, —(CH₂)₂NHC(O)(CH₂)₃—, —(CH₂)₂NHC(O)(CH₂)₄—, —(CH₂)₂NHC(O)(CH₂)₅—, —(CH₂)₃NHC(O)(CH₂)₃—, —(CH₂)₃NHC(O)(CH₂)₄—, —(CH₂)₄NHC(O)(CH₂)₄—, —(CH₂)₅NHC(O)(CH₂)₅—, —(CH₂)₂NHC(O)(CH₂)₂—O—(CH₂)₂—, —(CH₂)₄NHC(O)CH₂—, —CH₂-phenylene-CH₂—, —CH₂-phenylene-(CH₂)₂—, —CH₂-phenylene-(CH₂)₃—, —CH₂-phenylene-(CH₂)₄—, —CH₂-phenylene-(CH₂)₅—, —CH₂-phenylene-(CH₂)₆—, —(CH₂)₂-phenylene-(CH₂)₁—, —(CH₂)₂-phenylene-(CH₂)₂—, —(CH₂)₂-phenylene- (CH₂)₃—, —(CH₂)₂-phenylene-(CH₂)₄—, —(CH₂)₂-phenylene-(CH₂)₅—, —(CH₂)₃-phenylene-CH₂—, —(CH₂)₃-phenylene- (CH₂)₂—, —(CH₂)₃-phenylene-(CH₂)₃—, —(CH₂)₃-phenylene-(CH₂)₄—, —(CH₂)₃-phenylene-(CH₂)₅—, —(CH₂)₄- phenylene-CH₂—, —(CH₂)₄-phenylene-(CH₂)₂—, —(CH₂)₄-phenylene-(CH₂)₃—, —(CH₂)₄-phenylene-(CH₂)₄—, —(CH₂)₄-phenylene- (CH₂)₅—, —CH₂-piperidinylene-CH₂—, —CH₂-piperidinylene-(CH₂)₂—, —CH₂-piperidinylene-(CH₂)₃—, —CH₂-piperidinylene-(CH₂)₄—, —CH₂-piperidinylene-(CH₂)₅—, —CH₂-piperidinylene-(CH₂)₆—, —(CH₂)₂-piperidinylene-(CH₂)₁—, —(CH₂)₂-piperidinylene-(CH₂)₂—, —(CH₂)₂-piperidinylene-(CH₂)₃—, —(CH₂)₂-piperidinylene-(CH₂)₄—, —(CH₂)₂-piperidinylene-(CH₂)₅—, —(CH₂)₃-piperidinylene-CH₂—, —(CH₂)₃-piperidinylene-(CH₂)₂—, —(CH₂)₃-piperidinylene-(CH₂)₃—, —(CH₂)₃-piperidinylene-(CH₂)₄—, —(CH₂)₃-piperidinylene-(CH₂)₅—, —CH₂-piperazinylene-CH₂—, —CH₂-piperazinylene-(CH₂)₂—, —CH₂-piperazinylene-(CH₂)₃—, —CH₂-piperazinylene-(CH₂)₄—, —CH₂-piperazinylene-(CH₂)₅—, —(CH₂)₂-piperazinylene-(CH₂)₁—, —(CH₂)₂-piperazinylene-(CH₂)₂—, —(CH₂)₂-piperazinylene-(CH₂)₃—, —(CH₂)₂-piperazinylene-(CH₂)₄—, —(CH₂)₂-piperazinylene-(CH₂)₅—, —(CH₂)₃-piperazinylene-CH₂—, —(CH₂)₃-piperazinylene-(CH₂)₂—, —(CH₂)₃-piperazinylene-(CH₂)₃—, —(CH₂)₃-piperazinylene-(CH₂)₄—, —(CH₂)₃-piperazinylene-(CH₂)₅—, —(CH₂)₄-piperazinylene-CH₂—, —(CH₂)₄-piperazinylene-(CH₂)₂—, —(CH₂)₄-piperazinylene-(CH₂)₃—, —(CH₂)₄-piperazinylene-(CH₂)₄—, —(CH₂)₄-piperazinylene-(CH₂)₅—, —(CH₂)₈-piperazinylene-CH₂—, —(CH₂)₈-piperazinylene-(CH₂)₂—, —(CH₂)₈-piperazinylene-(CH₂)₃—, —(CH₂)₈-piperazinylene-(CH₂)₄—, or —(CH₂)₈-piperazinylene-(CH₂)₅—, or LIN represents: 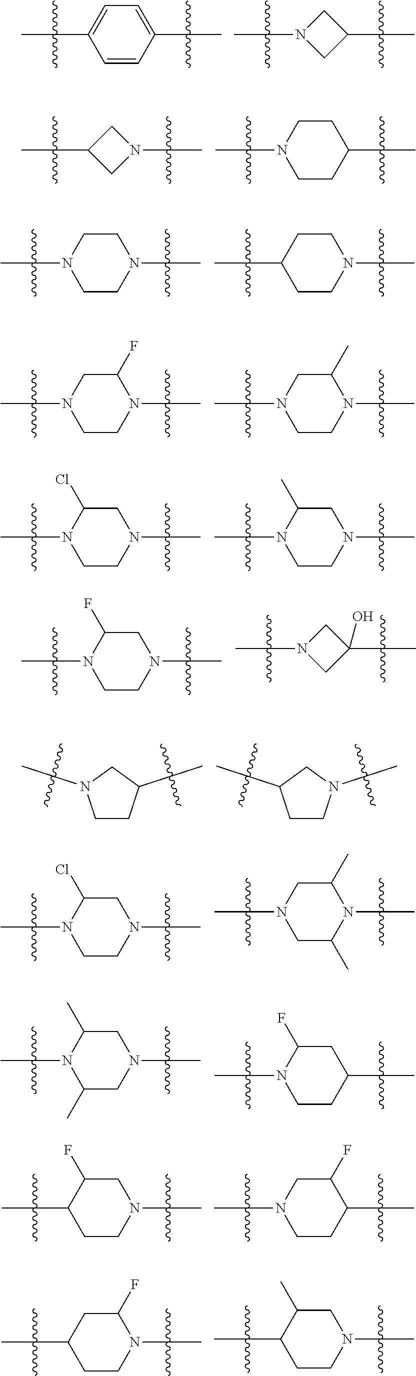 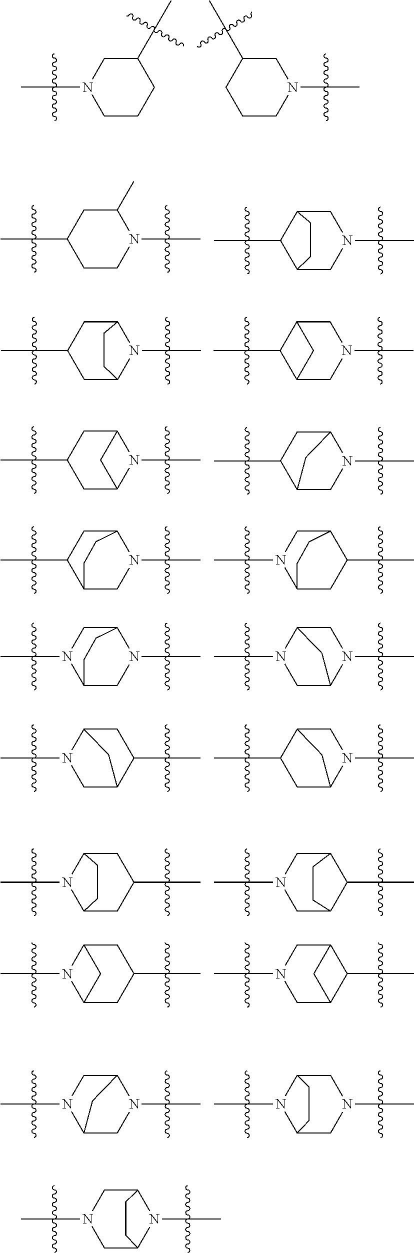 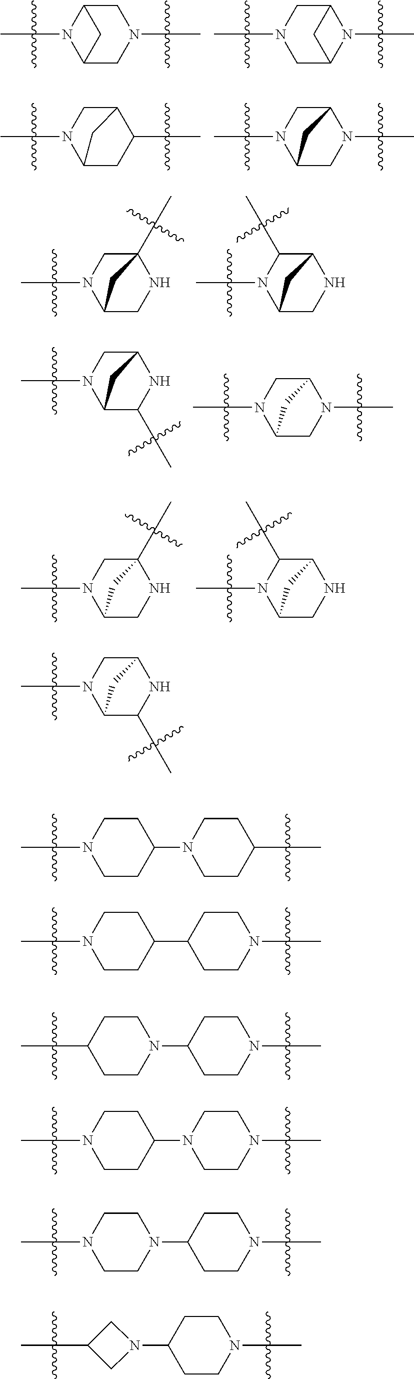 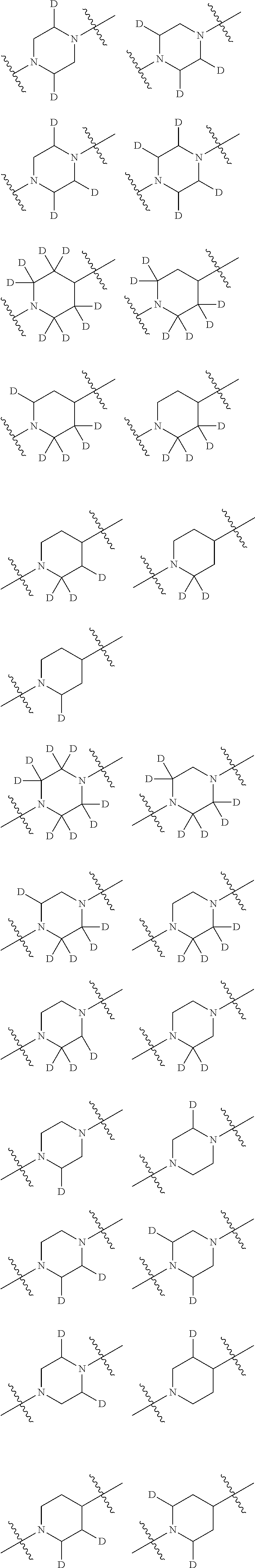 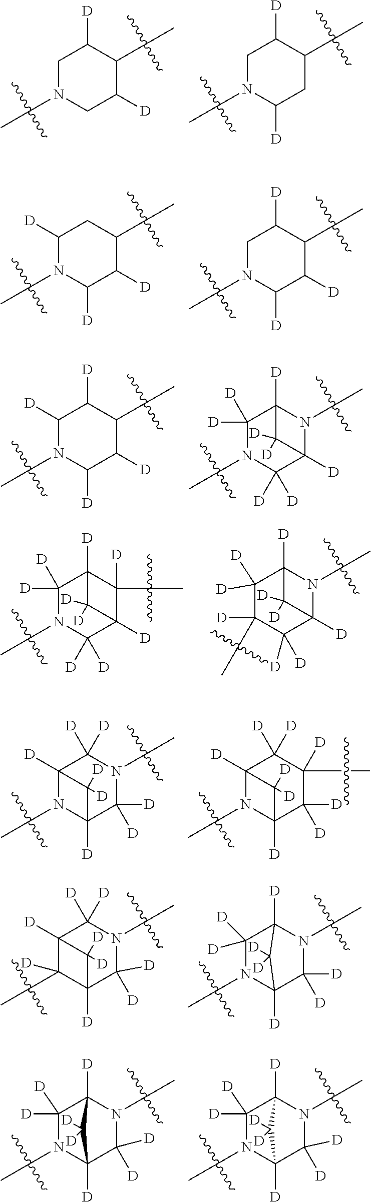 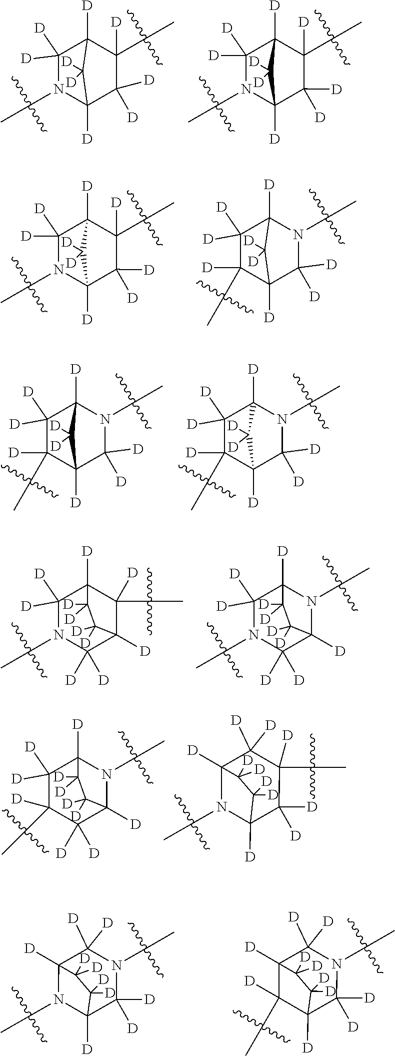 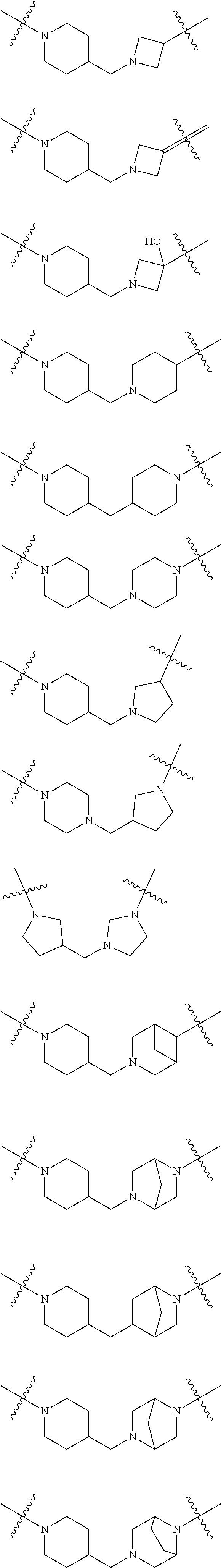 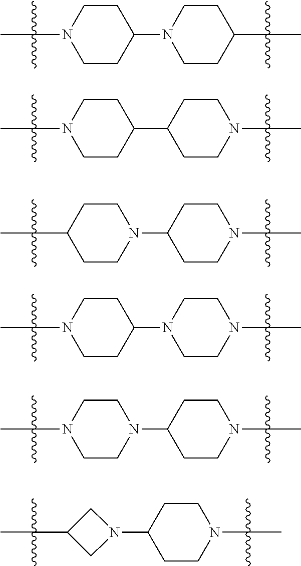 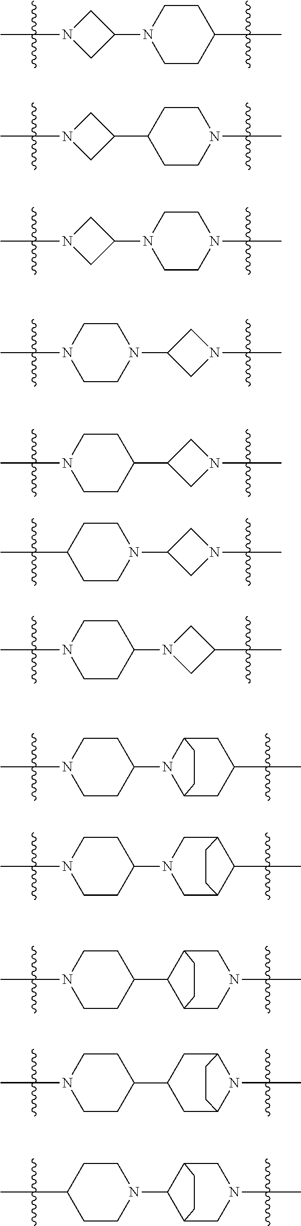 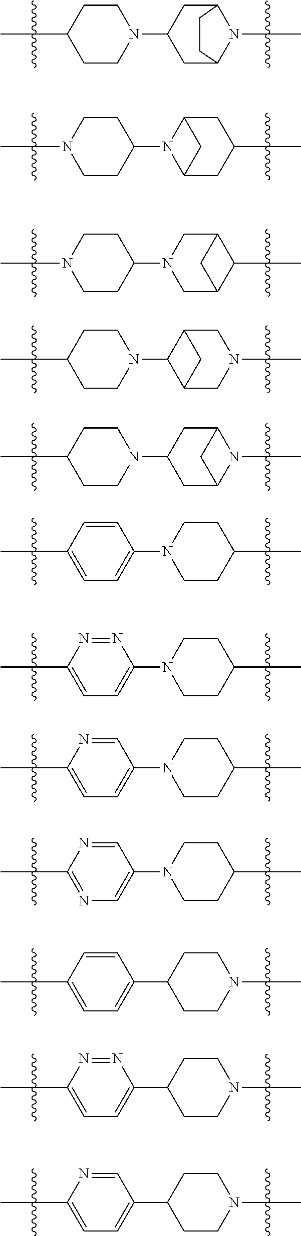 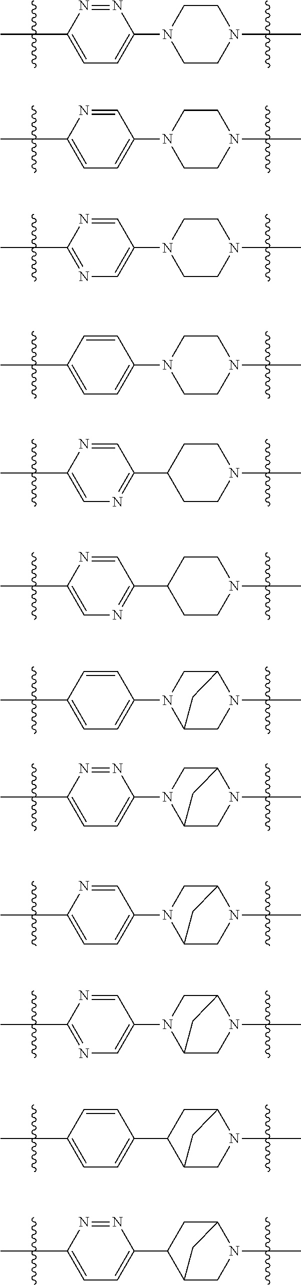 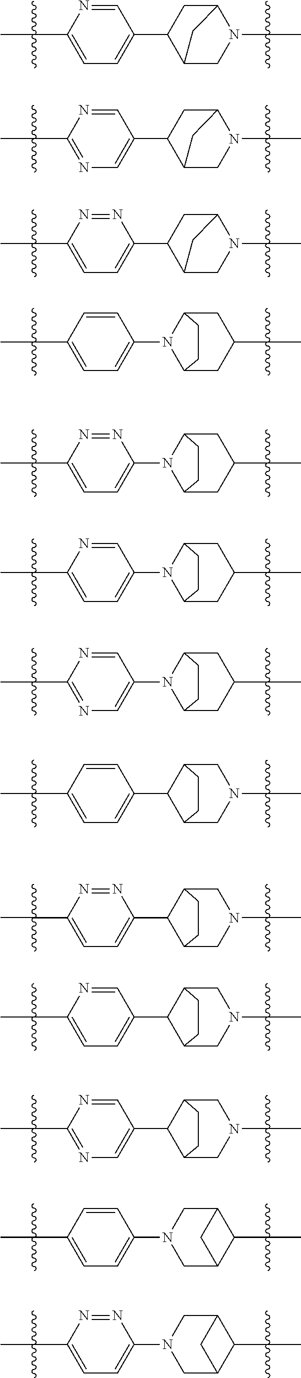 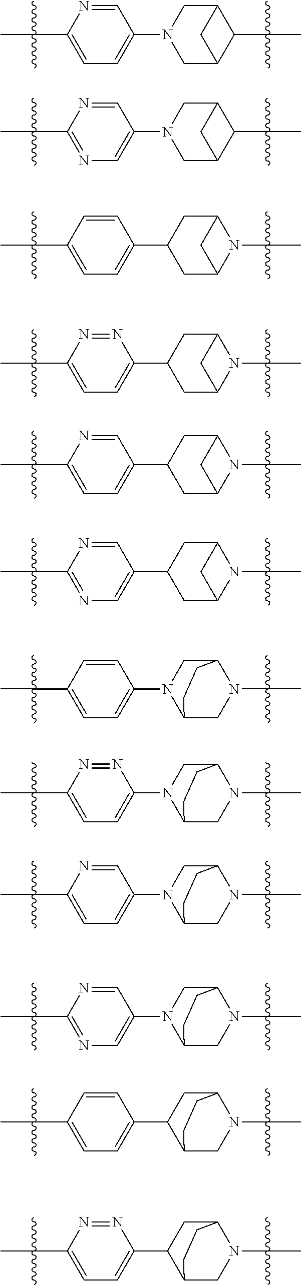 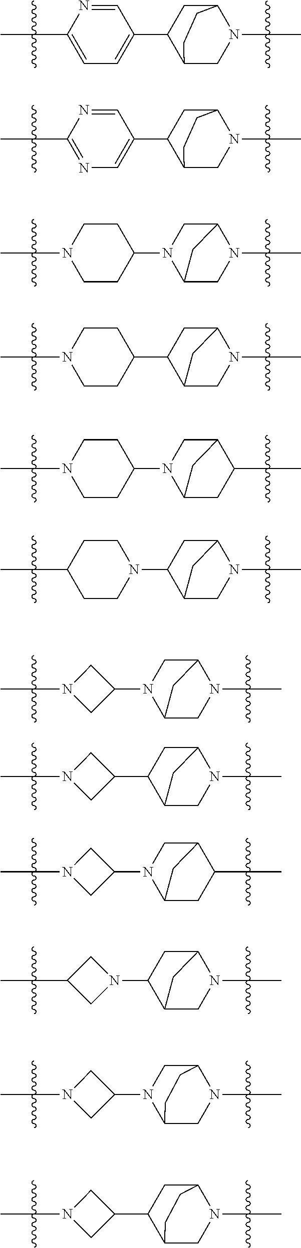 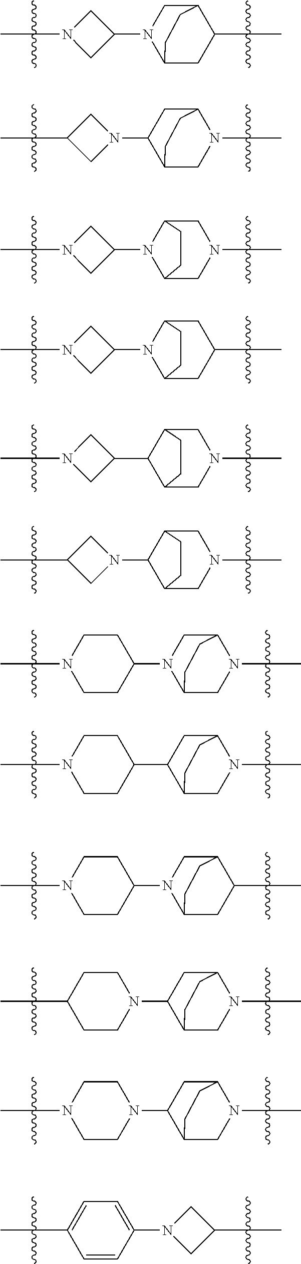 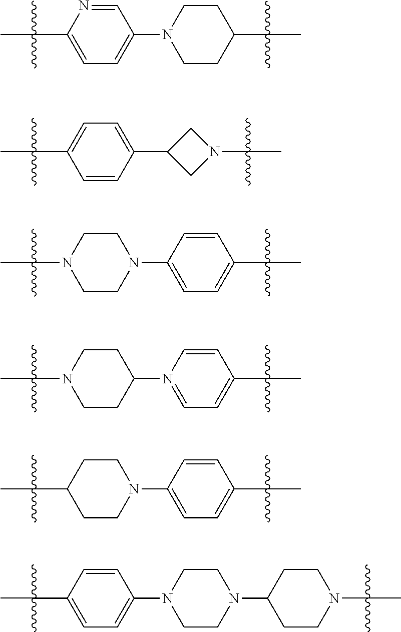 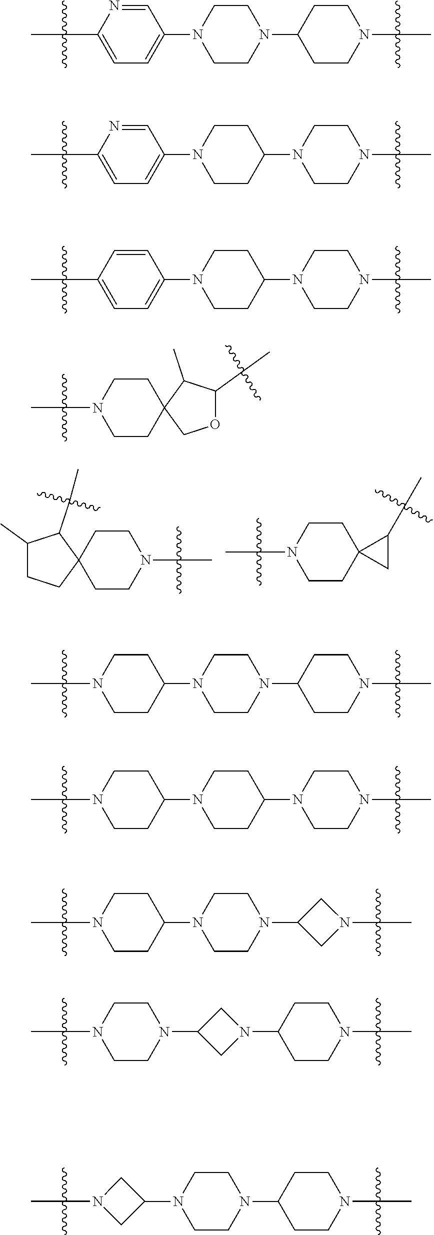 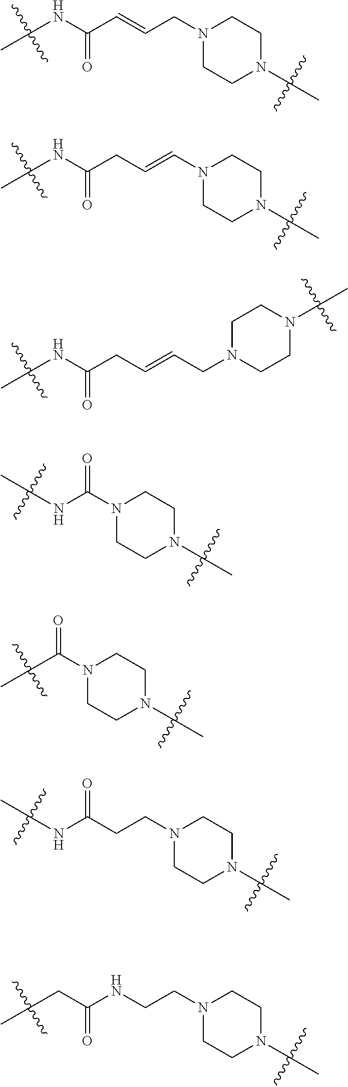 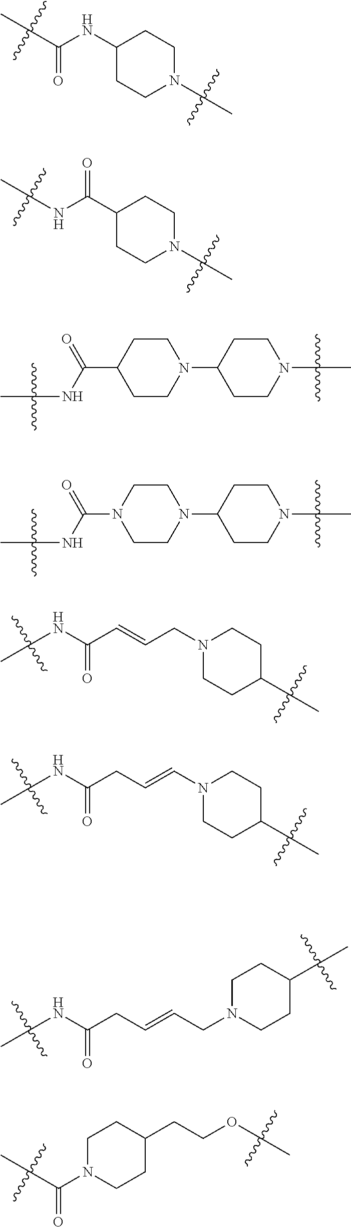 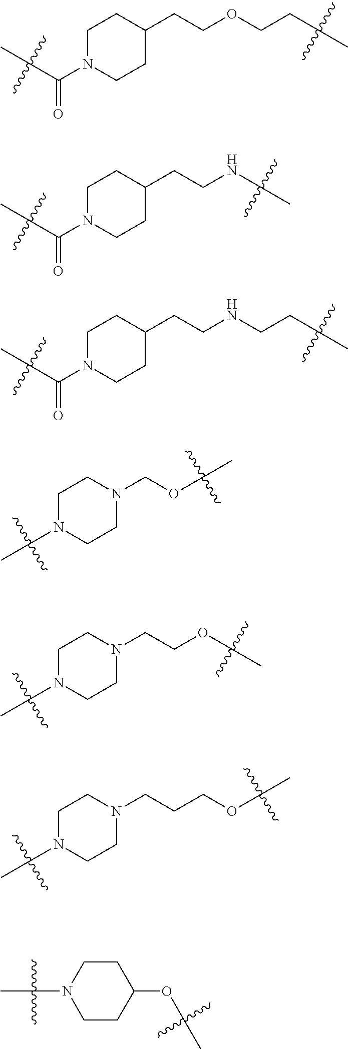 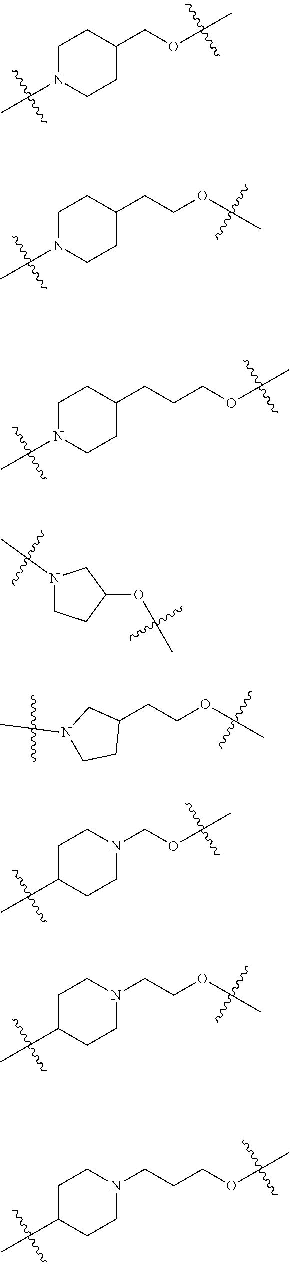 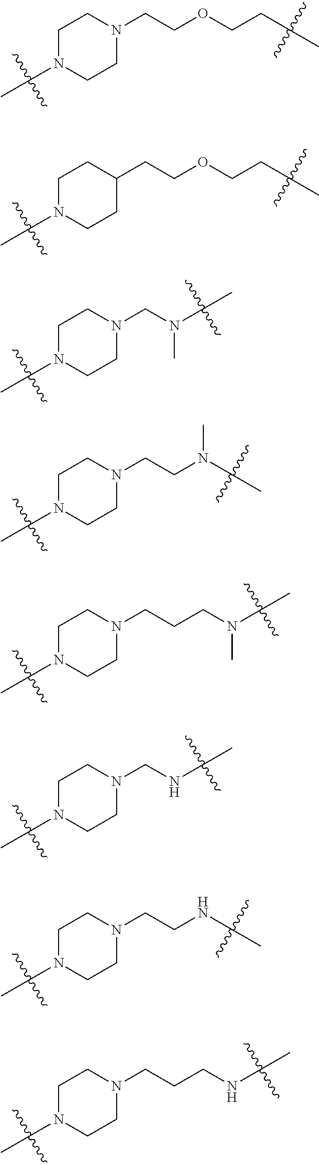 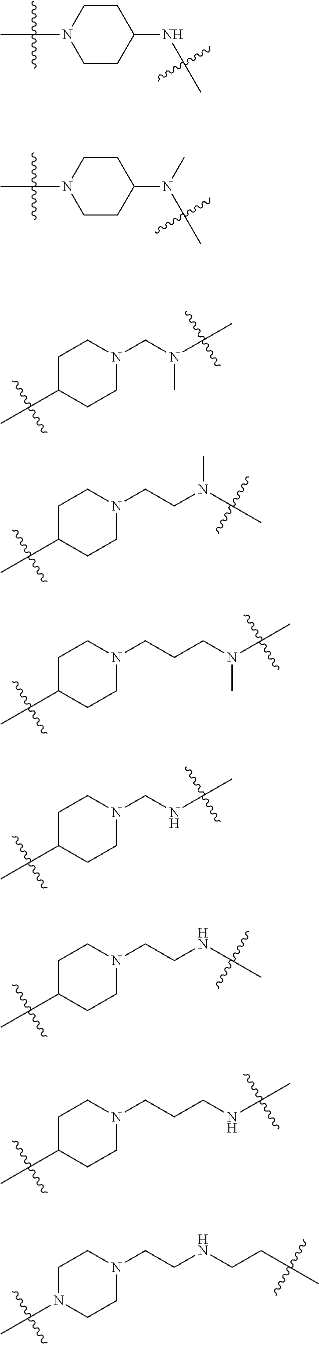 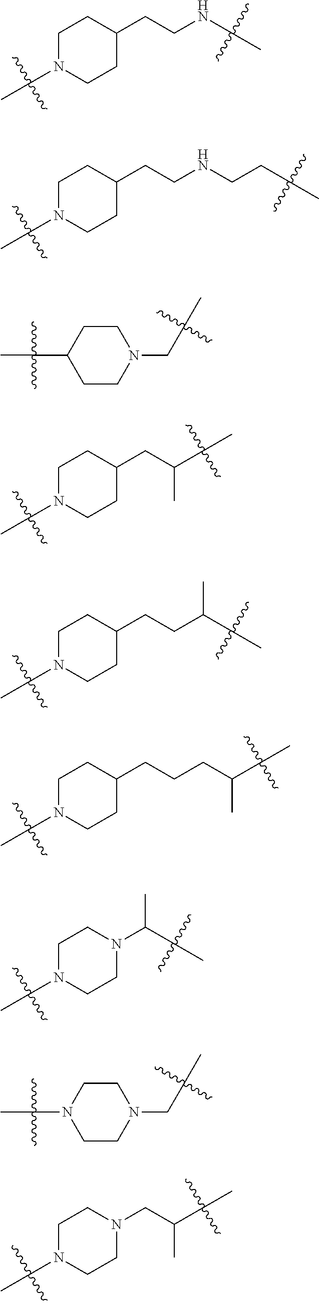 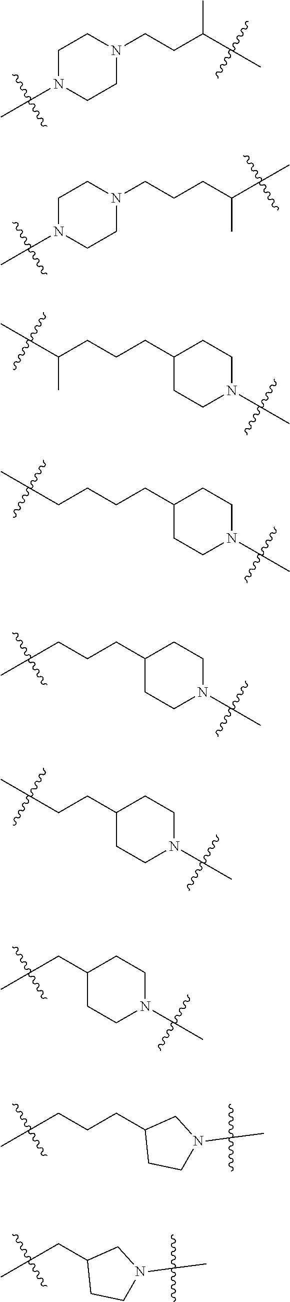 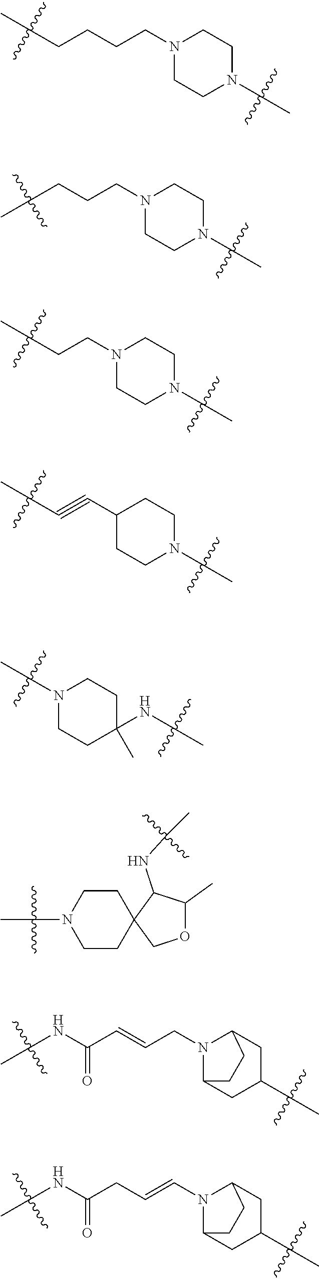 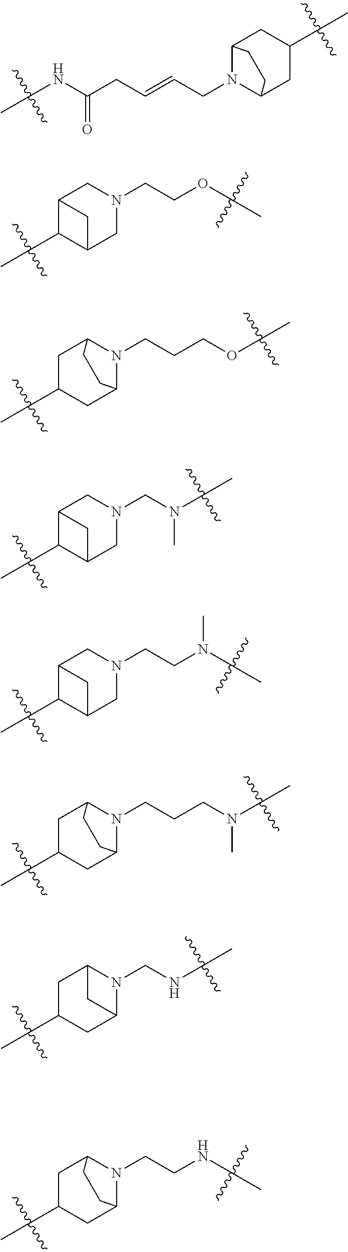 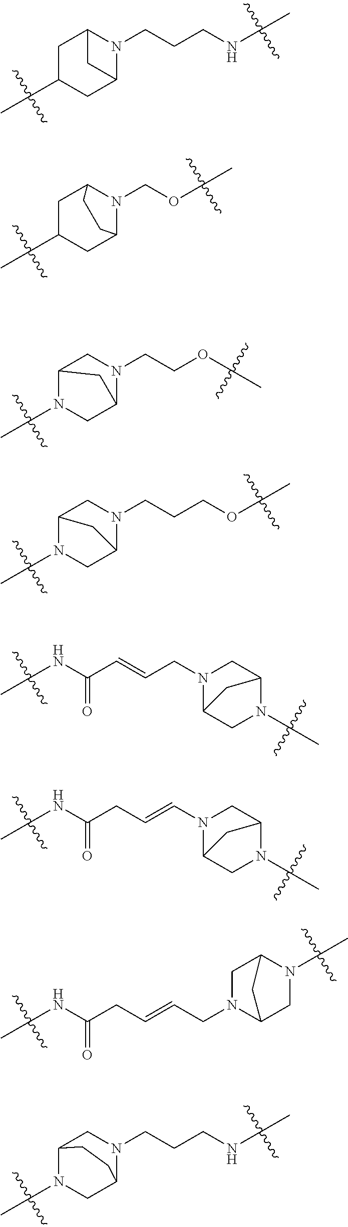 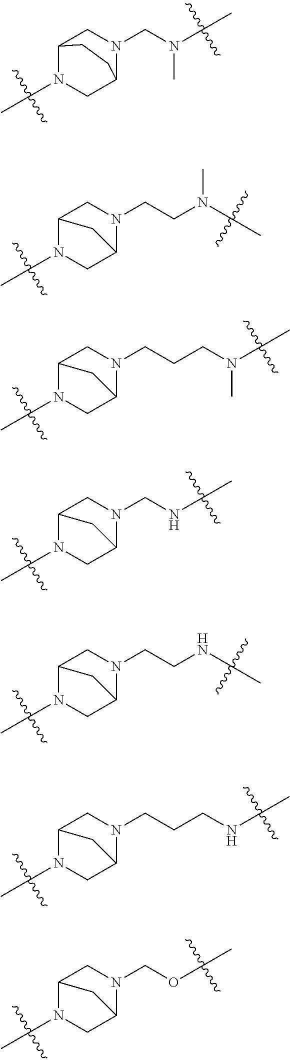 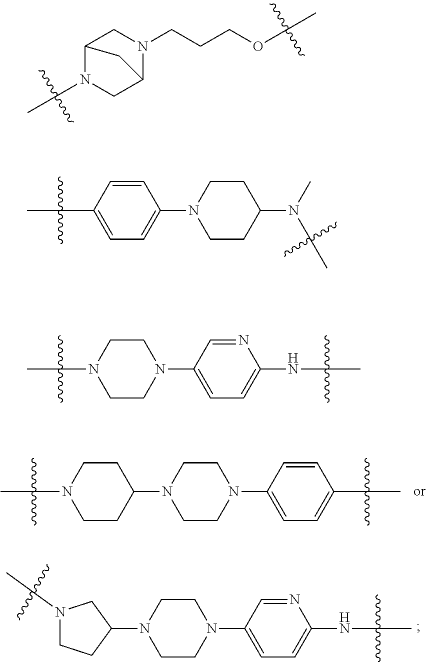 [SMILES: *OCCCN1CC2CC1CN2* ; *c1ccc(N2CCC(N(*)C)CC2)cc1 ; *Nc1ccc(N2CCN(*)CC2)cn1 ; *c1ccc(N2CCN(C3CCN(*)CC3)CC2)cc1 ; *Nc1ccc(N2CCN(C3CCN(*)C3)CC2)cn1]
wherein the groups are optionally substituted with a substituent selected from the group consisting of deuterium, C₁₋₆ alkyl, deuterated C₁₋₆ alkyl, halogenated C₁₋₆ alkyl, C₁₋₆ alkoxy, halogen, amino, hydroxy, mercapto, cyano, carboxyl, C₂₋₆ alkynyl, or C₂₋₆ alkenyl.

18. The compound of Formula (I) or salts, enantiomers, stereoisomers, solvates, isotopically enriched analogs, prodrugs, or polymorphs thereof as claimed in claim 3, which is also represented by Formula (I-1), Formula (I-2), Formula (I-3), Formula (I-4), Formula (I-5-1), Formula (I-5-2), Formula (I-5-3), Formula (I-5-4), Formula (I-6), Formula (I-7), Formula (I-8), Formula (I-9), Formula (I-10-1), Formula (I-10-2), Formula (I-11), Formula (I-12), Formula (I-13-1), Formula (I-13-2), Formula (I-14), Formula (I-15), Formula (I-16), Formula (I-17), Formula (I-18-1), Formula (I-18-2), Formula (I-19), Formula (I-20), Formula (I-21), Formula (I-22), Formula (I-23), Formula (I-24), Formula (I-25), Formula (I-26), Formula (I-27-1), Formula (I-27-2), Formula (I-28), Formula (I-29), Formula (I-30), Formula (I-31), or Formula (I-32): 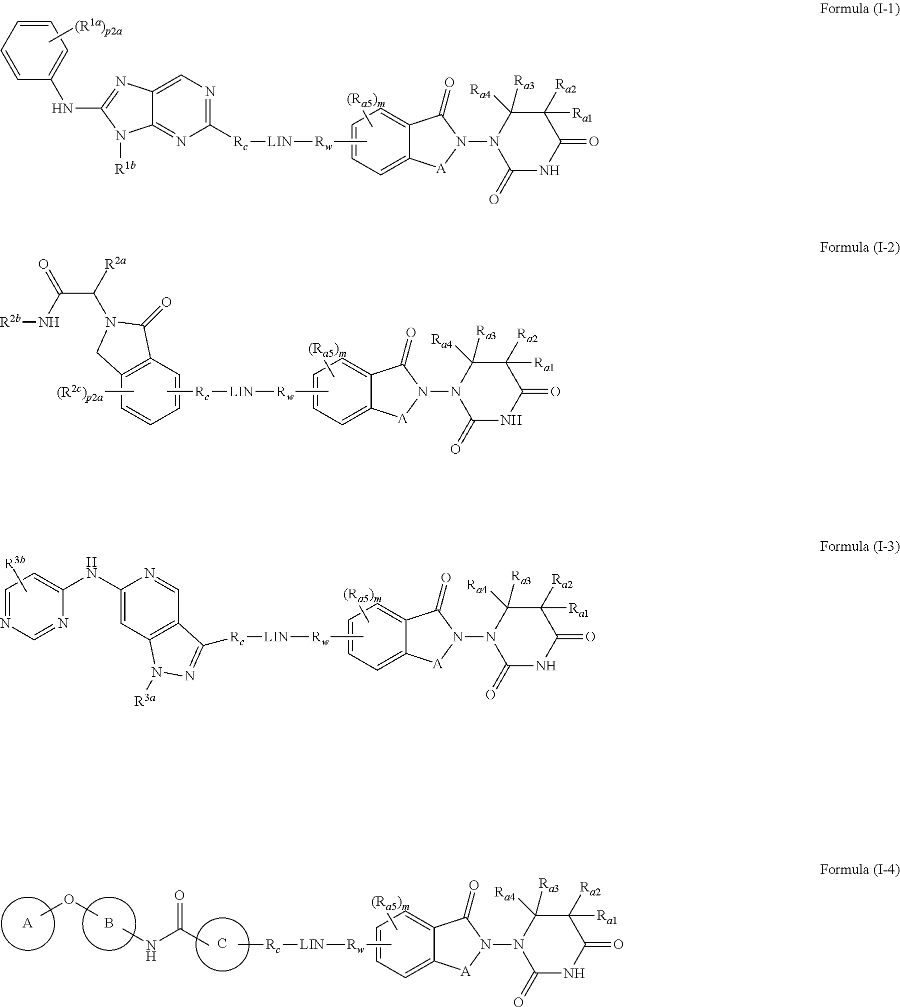 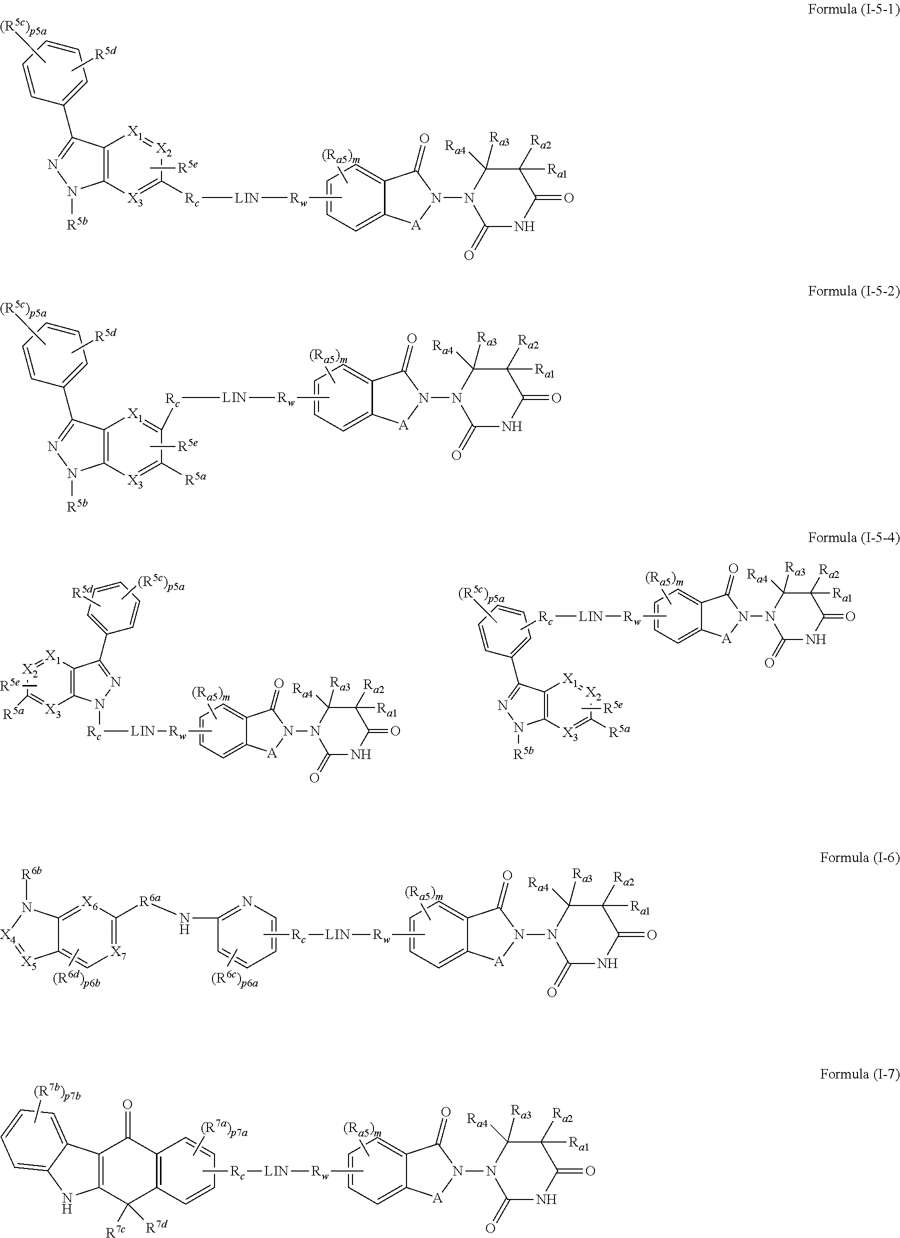 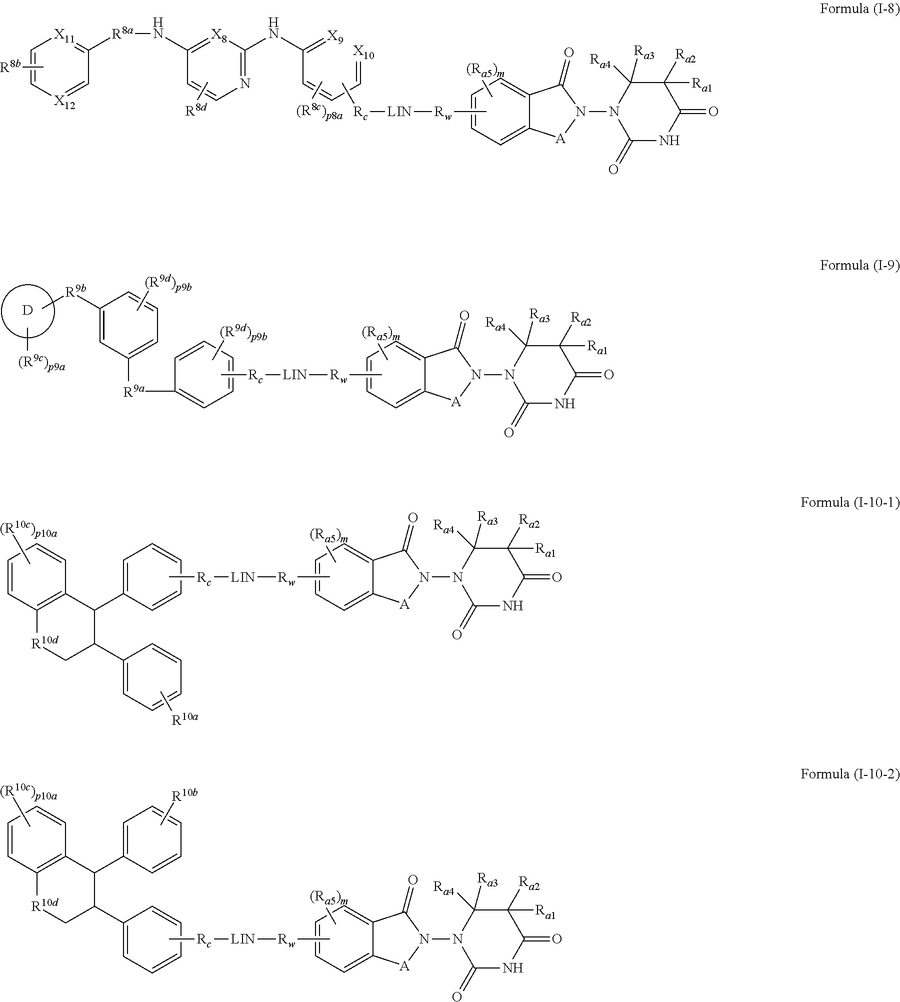 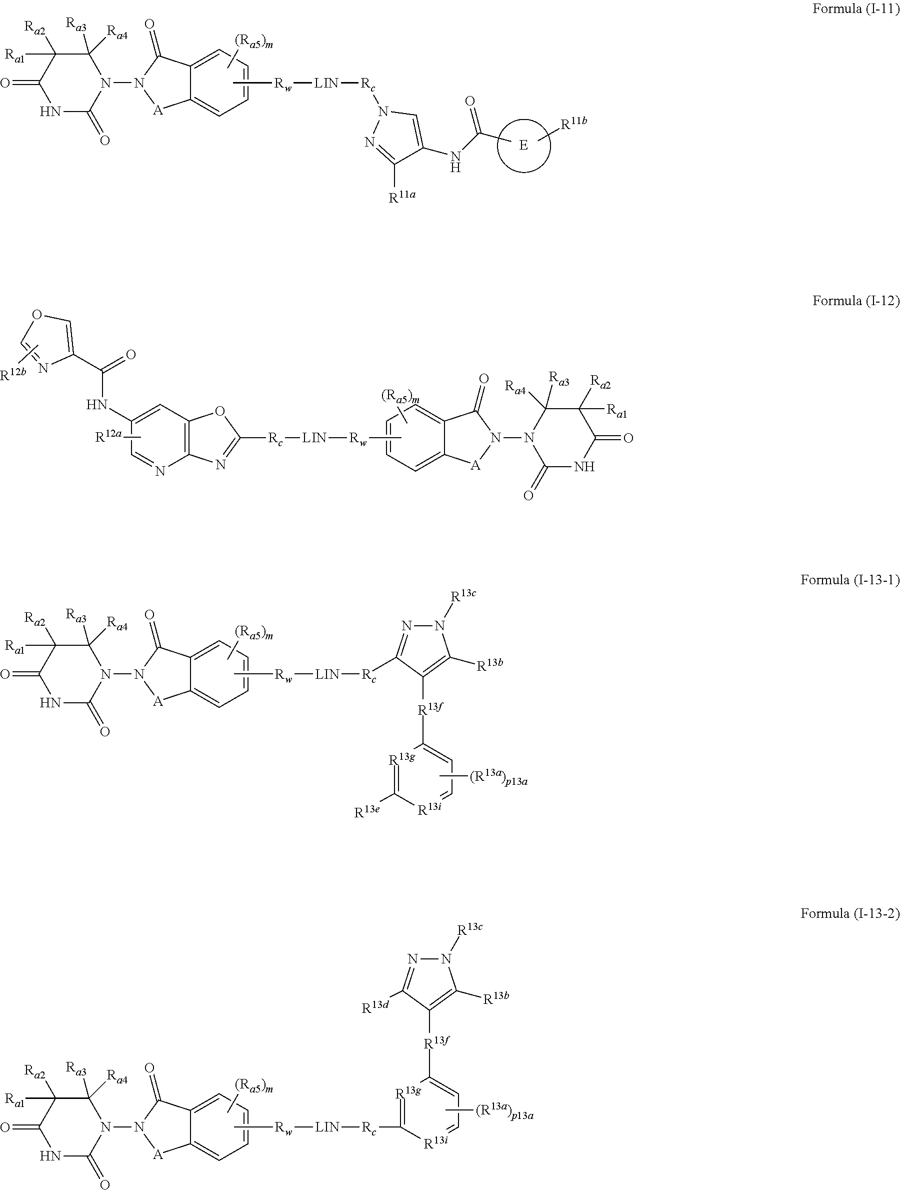 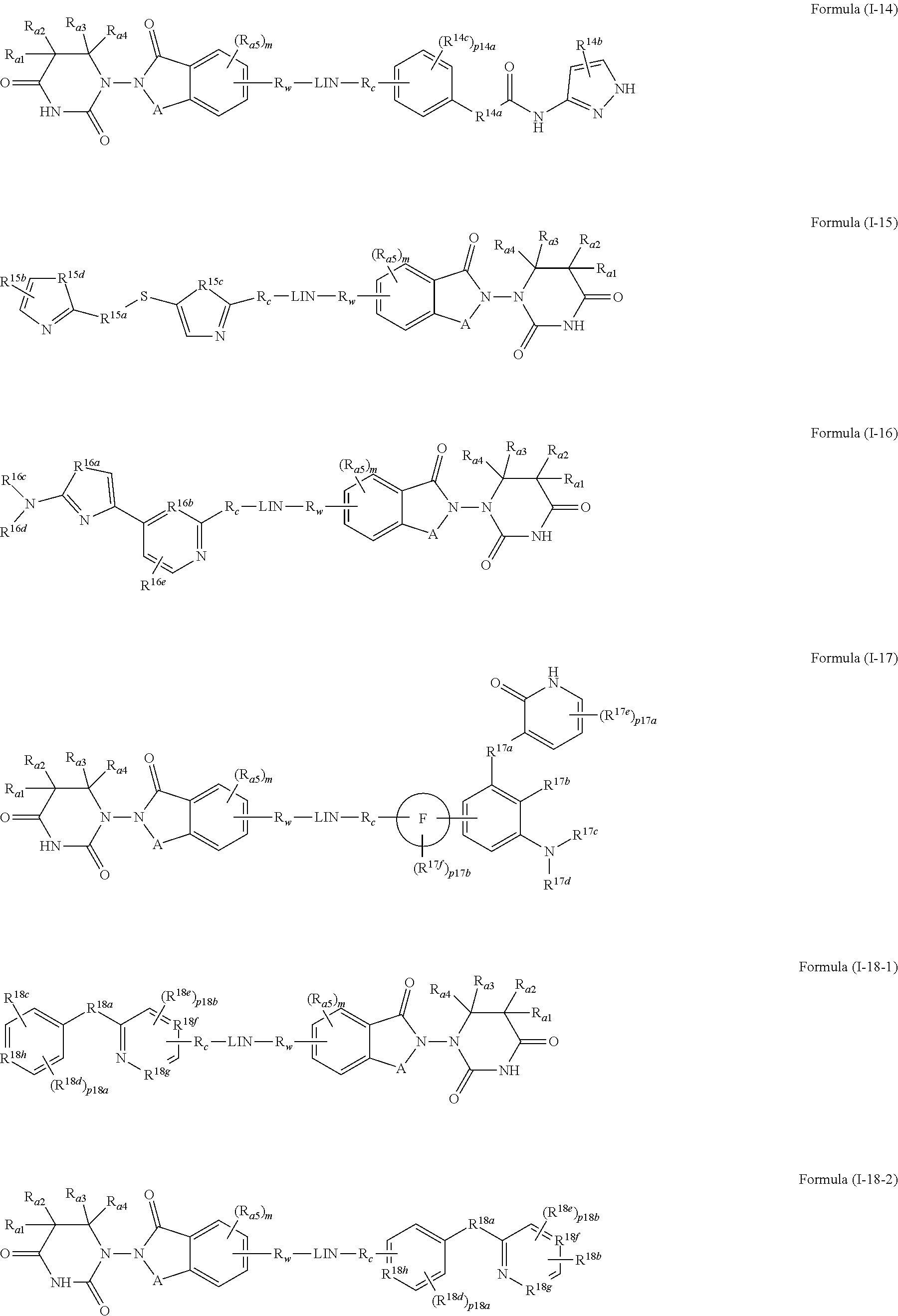 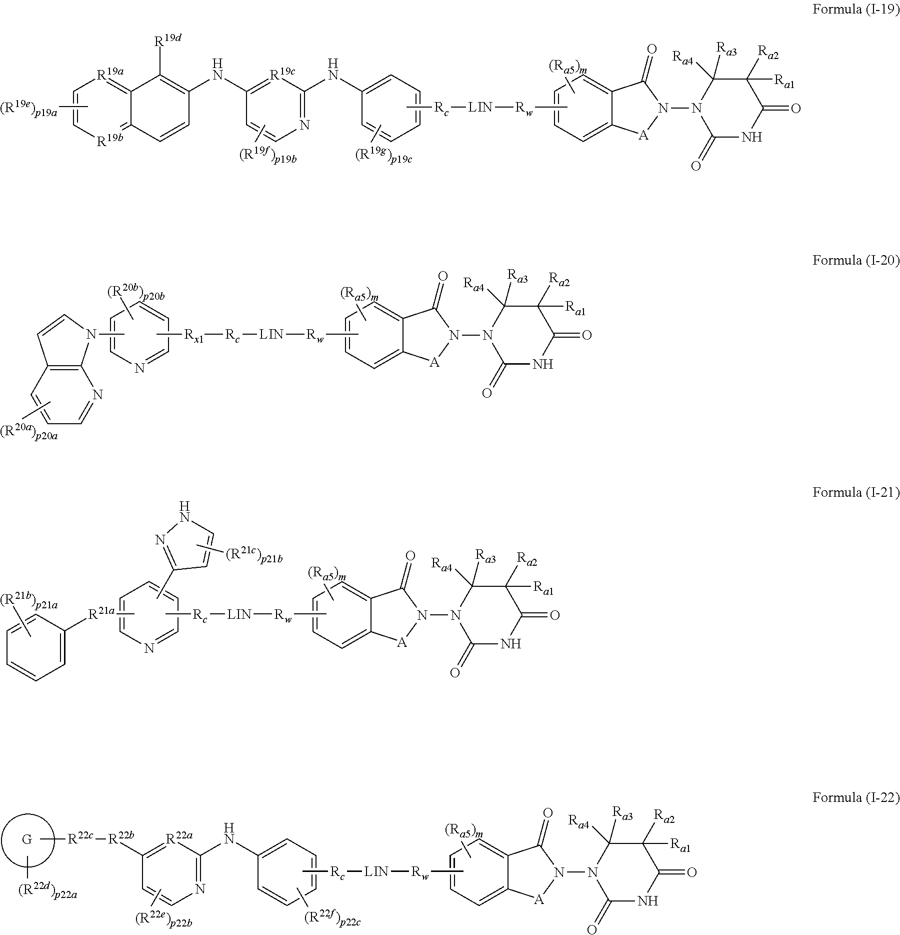 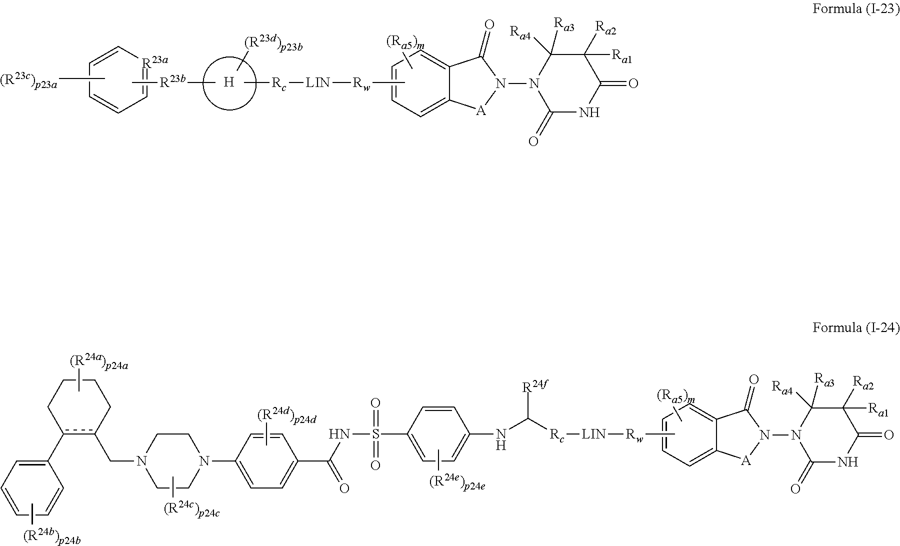 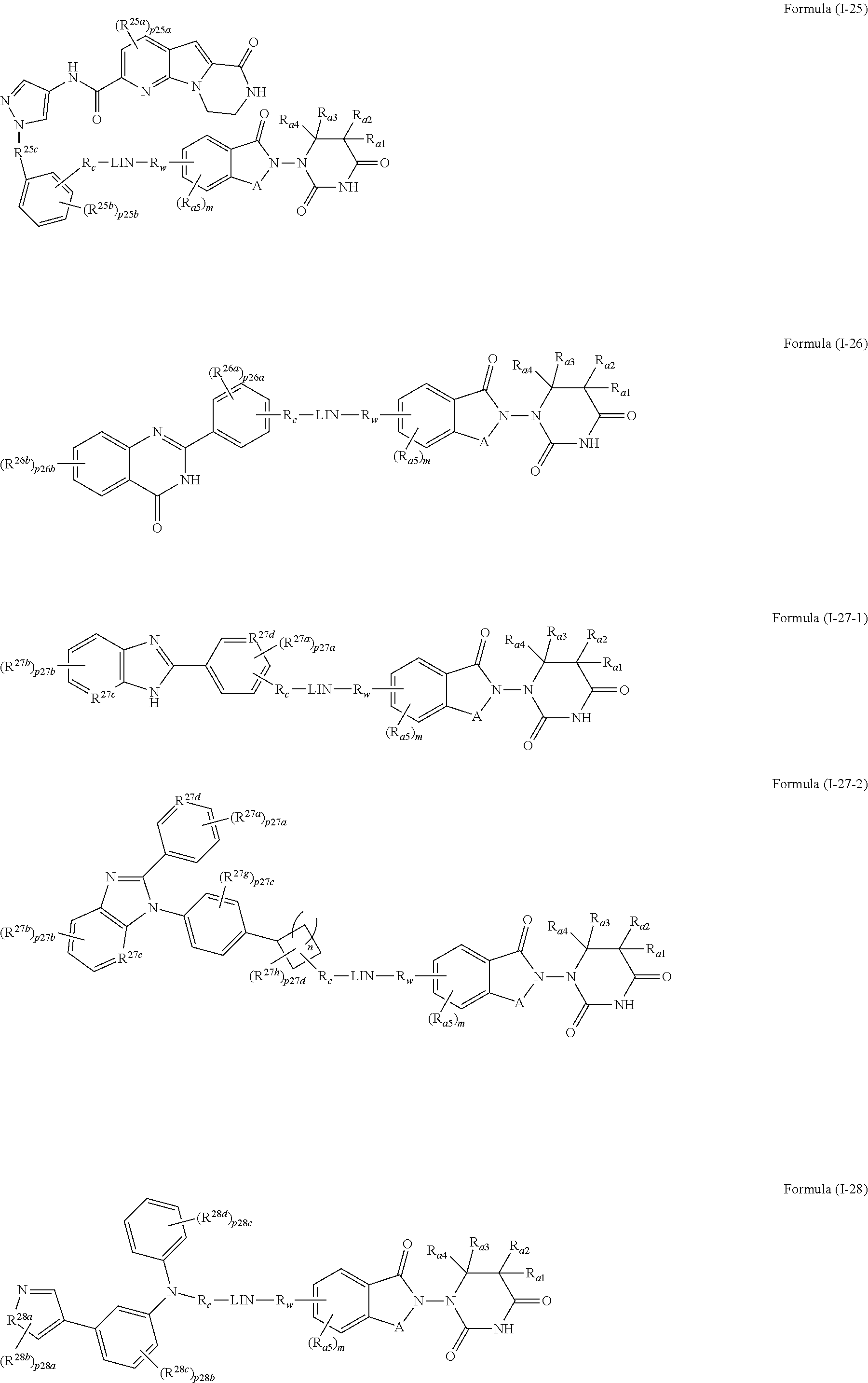 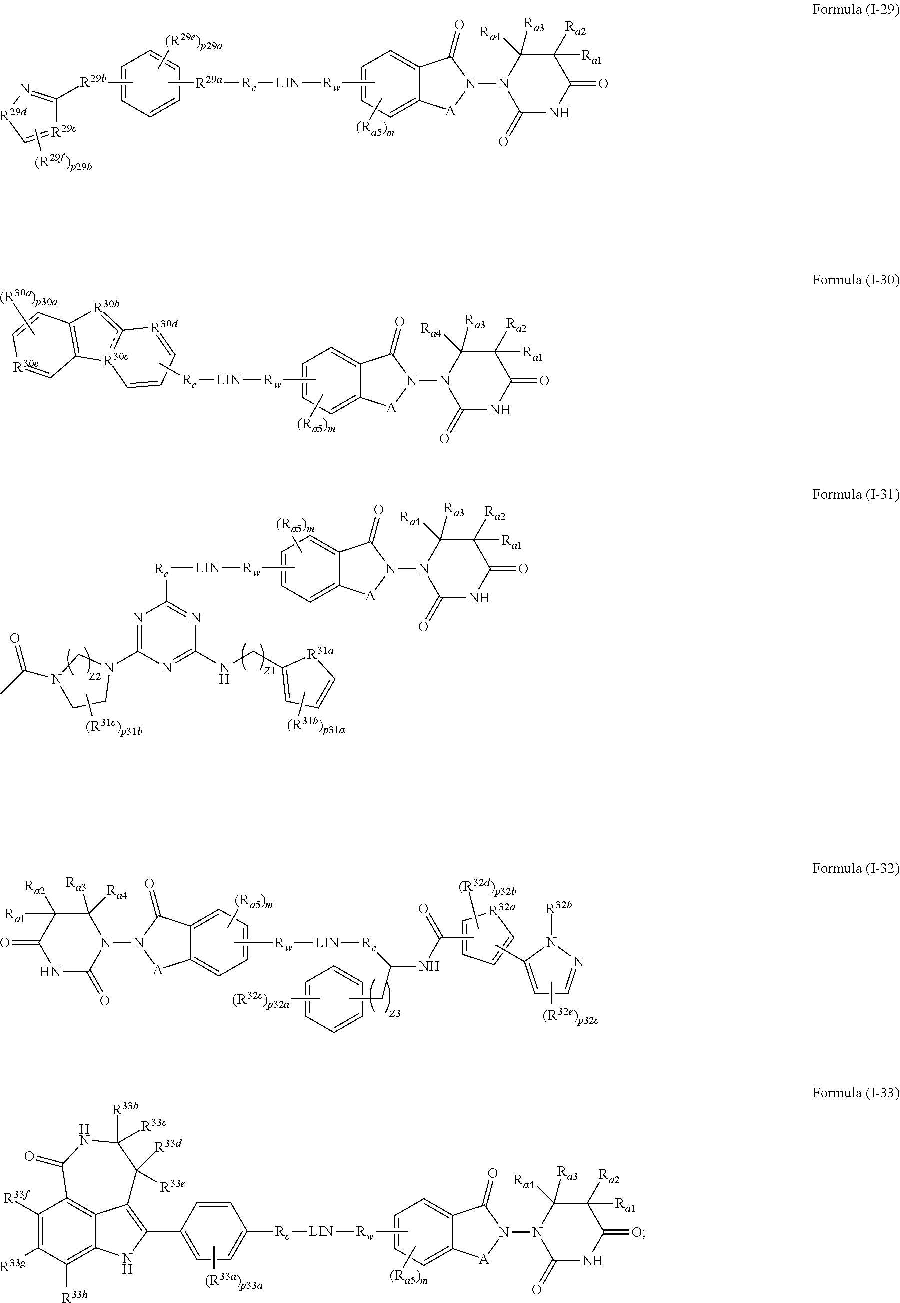
wherein A, Rₐ₁, Rₐ₂, Rₐ₃, Rₐ₄, (Rₐ₅) m, R_{w}, LIN, R_{c}, (R^{1a})ₚ₁ₐ, R^{1b}, R^{2a}, R^{2b}, (R^{2c})ₚ₂ₐ, R^{3a}, R^{3b}, ring A, ring B, ring C, R^{5a}, R^{5b}, (R^{5c})ₚ₅ₐ, R^{5d}, R^{5e}, X₁, X₂, X₃, R^{6a}, R^{6b}, (R^{6c})ₚ₆ₐ, (R^{6d})_{p6b}, X₄, X₅, X₆, X₇, (R^{7a})ₚ₇ₐ, (R^{7b})_{p7b}, R^{7c}, R^{7a}, R^{8a}, R^{8b}, (R^{8c})ₚ₈ₐ, R^{8d}, X₈, X₉, X₁₀, X₁₁, X₁₂, R^{9a}, R^{9b}, (R^{9c})ₚ₉ₐ, (R^{9d})_{p9b}, R^{10a}, R_{10b}, (R^{10c})ₚ₁₀ₐ, R^{10d}, R₁₁ₐ, R^{11b}, ring E, R^{12a}, R^{12b}, (R^{13a})ₚ₁₃ₐ, R^{13b}, R^{13c}, R^{13d}, R^{13e}, R^{13f}, R^{13g}, R¹³ⁱ, R^{14a}, R^{14b}, (R^{14c})ₚ₁₄ₐ, R^{15a}, R^{15b}, R^{15c}, R^{15d}, R^{16a}, R^{16b}, R^{16c}, R^{16d}, R^{16e}, R^{17a}, R^{17b}, R^{17c}, R^{17d}, (R^{17e})ₚ₁₇ₐ, (R^{17f})_{p17b}, ring F, R^{18a}, R^{18c}, (R^{18d})ₚ₁₈ₐ, (R^{18e})_{p18b}, R^{18f}, R^{18g}, R^{18h}, R^{19a}, R^{19b}, R^{19c}, R^{19d}, (R^{19e})ₚ₁₉ₐ, (R^{19f})_{p19b}, (R^{19g})_{p19c}, (R^{20a})ₚ₂₀ₐ, (R^{20b})_{p20b}, Rₓ₁, R^{21a}, (R^{21b})ₚ₂₁ₐ, (R^{21c})_{p21b}, R^{22a}, R^{22b}, R^{22c}, (R^{22d})ₚ₂₂ₐ, (R^{22e})_{p22b}, (R^{22f})_{p22c}, ring G, R^{23a}, R^{23b}, (R^{23c})ₚ₂₃ₐ, (R^{23d})_{p23b}, ring H, (R^{24a})ₚ₂₄ₐ, (R^{24b})_{p24b}, (R^{24c})_{p24c},n, (R^{24d})_{p24d}, (R^{24e})ₚ₂₄ₑ, R^{24f}, (R^{25a})ₚ₂₅ₐ, (R^{25b})_{p25b}, R^{25c}, (R^{26a})ₚ₂₆ₐ, (R^{26b})_{p26b}, (R^{27a})ₚ₂₇ₐ, (R^{27b})_{p27b}, R^{27c}, R^{27d}, (R^{27g})_{p27c}, n, (R^{27h})_{p27d}, R^{28a}, (R^{28b})ₚ₂₈ₐ, (R^{28c})_{p28b}, (R^{28d})_{p28c}, R^{29a}, R^{29b}, R^{29c}, R^{29d}, (R^{29e})ₚ₂₉ₐ, (R^{29f})_{p29b}, (R^{30a})ₚ₃₀ₐ, [symbol] , fragment 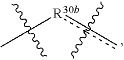
fragment 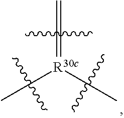
R^{30d}, R^{30e}, R^{31a}, (R^{31b})ₚ₃₁ₐ, (R^{31c})_{p31b}, Z1, Z2, R^{32a}, R^{32b}, (R^{32c})ₚ₃₂ₐ, (R^{32d})_{p32b}, (R^{32e})_{p32c}, Z3, (R^{33a})ₚ₃₃ₐ, R^{33b}, R^{33c}, R^{33d}, R^{33e}, R^{33f}, R^{33g}, and R^{33h} are as defined in claim 3.

19. The compound of Formula (I) or salts, enantiomers, stereoisomers, solvates, isotopically enriched analogs, prodrugs, or polymorphs thereof as claimed in claim 1, which is selected from:
1-(5-((4-(4-amino-3-(4-phenoxyphenyl)-1H-pyrazolo[3,4-d]pyrimidin-1-yl)piperidin-1-yl)methyl)-1-oxoisoindolin-2-yl)dihydropyrimidine-2,4 (1H,3H)-dione;
1-(5-((4-(4-((9-cyclopentyl-8-(phenylamino)-9H-purin-2-yl)amino)phenyl) piperazin-1-yl)methyl)-1-oxoisoindolin-2-yl)dihydropyrimidine-2,4 (1H,3H)-dione;
1-(5-(((1-(4-((9-cyclopentyl-8-(phenylamino)-9H-purin-2-yl)amino)phenyl) piperidin-4-yl)(methyl)amino)methyl)-1-oxoisoindolin-2-yl)dihydropyrimidine-2,4 (1H,3H)-dione;
1-(5-((4-(1-(4-((9-cyclopentyl-8-(phenylamino)-9H-purin-2-yl)amino)phenyl) piperidin-4-yl)piperazin-1-yl)methyl)-1-oxoisoindolin-2-yl)dihydropyrimidine-2,4 (1H,3H)-dione;
1-(5-((4-(4-((5-bromo-4-((5-(dimethylphosphoryl) quinoxalin-6-yl)amino)pyrimidin-2-yl)amino)-5-methoxy-2-methylphenyl) piperazin-1-yl)methyl)-1-oxoisoindolin-2-yl)dihydropyrimidine-2,4 (1H,3H)-dione;
1-(5-(((1-(4-((5-bromo-4-((5-(dimethylphosphoryl) quinoxalin-6-yl)amino)pyrimidin-2-yl)amino)-5-methoxy-2-methylphenyl) piperidin-4-yl)(methyl)amino)methyl)-1-oxoisoindolin-2-yl)dihydropyrimidine-2,4 (1H,3H)-dione;
1-(5-((4-(1-(4-((5-bromo-4-((5-(dimethylphosphoryl) quinoxalin-6-yl)amino)pyrimidin-2-yl)amino)-5-methoxy-2-methylphenyl) piperidin-4-yl)piperazin-1-yl)methyl)-1-oxoisoindolin-2-yl)dihydropyrimidine-2,4 (1H,3H)-dione;
2-(7-(((2-(2,4-dioxotetrahydropyrimidin-1 (2H)-yl)-1-oxoisoindolin-5-yl)methyl)amino)-1-oxoisoindolin-2-yl)-2-(5-fluoro-2-hydroxyphenyl)-N-(thiazol-2-yl)acetamide;
2-(6-(4-((2-(2,4-dioxotetrahydropyrimidin-1 (2H)-yl)-1-oxoisoindolin-5-yl)methyl)piperazin-1-yl)-1-oxoisoindolin-2-yl)-2-(5-fluoro-2-hydroxyphenyl)-N-(thiazol-2-yl)acetamide;
2-(6-(4-(((2-(2,4-dioxotetrahydropyrimidin-1 (2H)-yl)-1-oxoisoindolin-5-yl)methyl)(methyl)amino) piperidin-1-yl)-1-oxoisoindolin-2-yl)-2-(5-fluoro-2-hydroxyphenyl)-N-(thiazol-2-yl)acetamide;
2-(6-(4-(4-((2-(2,4-dioxotetrahydropyrimidin-1 (2H)-yl)-1-oxoisoindolin-5-yl)methyl)piperazin-1-yl)piperidin-1-yl)-1-oxoisoindolin-2-yl)-2-(5-fluoro-2-hydroxyphenyl)-N-(thiazol-2-yl)acetamide;
2-(6-(4-(4-((2-(2,4-dioxotetrahydropyrimidin-1 (2H)-yl)-1-oxoisoindolin-5-yl)methyl)piperazin-1-yl)phenyl)-1-oxoisoindolin-2-yl)-2-(5-fluoro-2-hydroxyphenyl)-N-(thiazol-2-yl)acetamide;
2-(6-(6-(4-((2-(2,4-dioxotetrahydropyrimidin-1 (2H)-yl)-1-oxoisoindolin-5-yl)methyl)piperazin-1-yl)pyridazin-3-yl)-1-oxoisoindolin-2-yl)-2-(5-fluoro-2-hydroxyphenyl)-N-(thiazol-2-yl)acetamide;
2-(6,7-dihydro-5H-pyrrolo[1,2-c]imidazol-1-yl)-2-(6-(4-(4-((2-(2,4-dioxotetrahydropyrimidin-1 (2H)-yl)-1-oxoisoindolin-5-yl)methyl)piperazin-1-yl)phenyl)-4-fluoro-1-oxoisoindolin-2-yl)-N-(thiazol-2-yl)acetamide;
2-(6,7-dihydro-5H-pyrrolo[1,2-c]imidazol-1-yl)-2-(6-(4-((1S,4S)-5-((2-(2,4-dioxotetrahydropyrimidin-1 (2H)-yl)-1-oxoisoindolin-5-yl)methyl)-2,5-diazabicyclo[2.2.1]heptan-2-yl)phenyl)-4-fluoro-1-oxoisoindolin-2-yl)-N-(thiazol-2-yl)acetamide;
2-(6,7-dihydro-5H-pyrrolo[1,2-c]imidazol-1-yl)-2-(6-(4-((1R,4R)-5-((2-(2,4-dioxotetrahydropyrimidin-1 (2H)-yl)-1-oxoisoindolin-5-yl)methyl)-2,5-diazabicyclo[2.2.1]heptan-2-yl)phenyl)-4-fluoro-1-oxoisoindolin-2-yl)-N-(thiazol-2-yl)acetamide;
2-(6,7-dihydro-5H-pyrrolo[1,2-c]imidazol-1-yl)-2-(6-(4-(8-((2-(2,4-dioxotetrahydropyrimidin-1 (2H)-yl)-1-oxoisoindolin-5-yl)methyl)-3,8-diazabicyclo[3.2.1]octan-3-yl)phenyl)-4-fluoro-1-oxoisoindolin-2-yl)-N-(thiazol-2-yl)acetamide;
2-(6,7-dihydro-5H-pyrrolo[1,2-c]imidazol-1-yl)-2-(6-(4-(4-(((2-(2,4-dioxotetrahydropyrimidin-1 (2H)-yl)-1-oxoisoindolin-5-yl)methyl)(methyl)amino) piperidin-1-yl)phenyl)-4-fluoro-1-oxoisoindolin-2-yl)-N-(thiazol-2-yl)acetamide;
2-(6,7-dihydro-5H-pyrrolo[1,2-c]imidazol-1-yl)-2-(6-(4-(4-(4-((2-(2,4-dioxotetrahydropyrimidin-1 (2H)-yl)-1-oxoisoindolin-5-yl)methyl)piperazin-1-yl)piperidin-1-yl)phenyl)-4-fluoro-1-oxoisoindolin-2-yl)-N-(thiazol-2-yl)acetamide;
2-(6,7-dihydro-5H-pyrrolo[1,2-c]imidazol-1-yl)-2-(6-(4-(4-(4-((2-(2,4-dioxotetrahydropyrimidin-1 (2H)-yl)-1-oxoisoindolin-5-yl)methyl)piperazin-1-yl)-3,3-difluoropiperidin-1-yl)phenyl)-4-fluoro-1-oxoisoindolin-2-yl)-N-(thiazol-2-yl)acetamide;
2-(6,7-dihydro-5H-pyrrolo[1,2-c]imidazol-1-yl)-2-(6-(4-(4-(1-((2-(2,4-dioxotetrahydropyrimidin-1 (2H)-yl)-1-oxoisoindolin-5-yl)methyl)piperidin-4-yl)piperazin-1-yl)phenyl)-4-fluoro-1-oxoisoindolin-2-yl)-N-(thiazol-2-yl)acetamide;
2-(6,7-dihydro-5H-pyrrolo[1,2-c]imidazol-1-yl)-2-(6-(4-(4-(1-((2-(2,4-dioxotetrahydropyrimidin-1 (2H)-yl)-1-oxoisoindolin-5-yl)methyl)-3,3-difluoropiperidin-4-yl)piperazin-1-yl)phenyl)-4-fluoro-1-oxoisoindolin-2-yl)-N-(thiazol-2-yl)acetamide;
1-(5-((4-(6-((2-(1-(cyclopropylsulfonyl)-1H-pyrazol-4-yl)pyrimidin-4-yl)amino)-1-isopropyl-1H-pyrazolo[4,3-c]pyridin-3-yl)piperazin-1-yl)methyl)-1-oxoisoindolin-2-yl)dihydropyrimidine-2,4 (1H,3H)-dione;
1-(5-(((1-(6-((2-(1-(cyclopropylsulfonyl)-1H-pyrazol-4-yl)pyrimidin-4-yl)amino)-1-isopropyl-1H-pyrazolo[4,3-c]pyridin-3-yl)piperidin-4-yl)(methyl)amino)methyl)-1-oxoisoindolin-2-yl)dihydropyrimidine-2,4 (1H,3H)-dione;
1-(5-((4-(1-(6-((2-(1-(cyclopropylsulfonyl)-1H-pyrazol-4-yl)pyrimidin-4-yl)amino)-1-isopropyl-1H-pyrazolo[4,3-c]pyridin-3-yl)piperidin-4-yl)piperazin-1-yl)methyl)-1-oxoisoindolin-2-yl)dihydropyrimidine-2,4 (1H,3H)-dione;
1-(5-((4-(1-isopropyl-6-((2-(4-methoxypiperidin-1-yl)pyrimidin-4-yl)amino)-1H-pyrazolo[4,3-c]pyridin-3-yl) piperazin-1-yl)methyl)-1-oxoisoindolin-2-yl)dihydropyrimidine-2,4 (1H,3H)-dione;
1-(5-(((1-(1-isopropyl-6-((2-(4-methoxypiperidin-1-yl)pyrimidin-4-yl)amino)-1H-pyrazolo[4,3-c]pyridin-3-yl) piperidin-4-yl)(methyl)amino)methyl)-1-oxoisoindolin-2-yl)dihydropyrimidine-2,4 (1H,3H)-dione;
1-(5-((4-(1-(1-isopropyl-6-((2-(4-methoxypiperidin-1-yl)pyrimidin-4-yl)amino)-1H-pyrazolo[4,3-c]pyridin-3-yl) piperidin-4-yl)piperazin-1-yl)methyl)-1-oxoisoindolin-2-yl)dihydropyrimidine-2,4 (1H,3H)-dione;
N-(5-(((5-(tert-butyl) oxazol-2-yl)methyl)thio) thiazol-2-yl)-1-((2-(2,4-dioxotetrahydropyrimidin-1 (2H)-yl)-1-oxoisoindolin-5-yl)methyl)piperidine-4-carboxamide;
N-(5-(((5-(tert-butyl) oxazol-2-yl)methyl)thio) thiazol-2-yl)-1′-((2-(2,4-dioxotetrahydropyrimidin-1 (2H)-yl)-1-oxoisoindolin-5-yl)methyl)-[1,4′-bipiperidine]-4-carboxamide;
4-(2,6-dichlorobenzamido)-N-(1-((2-(2,4-dioxotetrahydropyrimidin-1 (2H)-yl)-1-oxoisoindolin-5-yl)methyl)piperidin-4-yl)-1H-pyrazole-3-carboxamide;
N-(5-cyclobutyl-1H-pyrazol-3-yl)-2-(4-((2-(2,4-dioxotetrahydropyrimidin-1 (2H)-yl)-1-oxoisoindolin-5-yl)methoxy)phenyl)acetamide;
1-(5-((4-((5-chloro-4-(5-(cyclopropylmethyl)-1-methyl-1H-pyrazol-4-yl)pyrimidin-2-yl)amino) piperidin-1-yl)methyl)-1-oxoisoindolin-2-yl)dihydropyrimidine-2,4 (1H,3H)-dione;
4-(((4-(5-chloro-2-((1-((2-(2,4-dioxotetrahydropyrimidin-1 (2H)-yl)-1-oxoisoindolin-5-yl)methyl)piperidin-4-yl)amino)pyridin-4-yl) thiazol-2-yl)amino)methyl)tetrahydro-2H-pyran-4-carbonitrile;
4-(((5′-chloro-2′-((1-((2-(2,4-dioxotetrahydropyrimidin-1 (2H)-yl)-1-oxoisoindolin-5-yl)methyl)piperidin-4-yl)amino)-[2,4′-bipyridin]-6-yl)amino)methyl)tetrahydro-2H-pyran-4-carbonitrile;
4-(((5′-chloro-2′-((1-((2-(2,4-dioxotetrahydropyrimidin-1 (2H)-yl)-1-oxoisoindolin-4-yl)methyl)piperidin-4-yl)amino)-[2,4′-bipyridin]-6-yl)amino)methyl)tetrahydro-2H-pyran-4-carbonitrile;
N-(4-(chlorodifluoromethoxy)phenyl)-6-(4-((2-(2,4-dioxotetrahydropyrimidin-1 (2H)-yl)-1-oxoisoindolin-5-yl)methyl)piperazin-1-yl)-5-(1H-pyrazol-3-yl)nicotinamide;
N-(4-(chlorodifluoromethoxy)phenyl)-6-(4-(((2-(2,4-dioxotetrahydropyrimidin-1 (2H)-yl)-1-oxoisoindolin-5-yl)methyl)(methyl)amino) piperidin-1-yl)-5-(1H-pyrazol-3-yl)nicotinamide;
N-(4-(chlorodifluoromethoxy)phenyl)-6-(4-(1-((2-(2,4-dioxotetrahydropyrimidin-1 (2H)-yl)-1-oxoisoindolin-5-yl)methyl)piperidin-4-yl)piperazin-1-yl)-5-(1H-pyrazol-3-yl)nicotinamide;
N-(4-(chlorodifluoromethoxy)phenyl)-6-(4-(4-((2-(2,4-dioxotetrahydropyrimidin-1 (2H)-yl)-1-oxoisoindolin-5-yl)methyl)piperazin-1-yl)piperidin-1-yl)-5-(1H-pyrazol-3-yl)nicotinamide;
N-(4-((4-((2-(2,4-dioxotetrahydropyrimidin-1 (2H)-yl)-1-oxoisoindolin-5-yl)methyl)piperazin-1-yl)methyl)-3-(trifluoromethyl)phenyl)-3-(imidazo[1,2-b]pyridazin-3-ylethynyl)-4-methylbenzamide;
N-(4-(4-((2-(2,4-dioxotetrahydropyrimidin-1 (2H)-yl)-1-oxoisoindolin-5-yl)methyl)piperazin-1-yl)-3-(trifluoromethyl)phenyl)-3-(imidazo[1,2-b]pyridazin-3-ylethynyl)-4-methylbenzamide;
N-(4-(4-(1-((2-(2,4-dioxotetrahydropyrimidin-1 (2H)-yl)-1-oxoisoindolin-5-yl)methyl)piperidin-4-yl) piperazin-1-yl)-3-(trifluoromethyl)phenyl)-3-(imidazo[1,2-b]pyridazin-3-ylethynyl)-4-methylbenzamide;
N-(4-(4-(((2-(2,4-dioxotetrahydropyrimidin-1 (2H)-yl)-1-oxoisoindolin-5-yl)methyl)(methyl)amino) piperidin-1-yl)-3-(trifluoromethyl)phenyl)-3-(imidazo[1,2-b]pyridazin-3-ylethynyl)-4-methylbenzamide;
7-cyclopentyl-2-((5-(4-((2-(2,4-dioxotetrahydropyrimidin-1 (2H)-yl)-1-oxoisoindolin-5-yl)methyl)piperazin-1-yl)pyridin-2-yl)amino)-N,N-dimethyl-7H-pyrrolo[2,3-d]pyrimidine-6-carboxamide;
7-cyclopentyl-2-((5-(4-((4-(2-(2,4-dioxotetrahydropyrimidin-1 (2H)-yl)-1-oxoisoindolin-5-yl)piperazin-1-yl)methyl)piperidin-1-yl)pyridin-2-yl)amino)-N,N-dimethyl-7H-pyrrolo[2,3-d]pyrimidine-6-carboxamide;
7-cyclopentyl-2-((5-(4-(((2-(2,4-dioxotetrahydropyrimidin-1 (2H)-yl)-1-oxoisoindolin-5-yl)methyl)(methyl)amino) piperidin-1-yl)pyridin-2-yl)amino)-N,N-dimethyl-7H-pyrrolo[2,3-d]pyrimidine-6-carboxamide;
7-cyclopentyl-2-((5-(4-(4-((2-(2,4-dioxotetrahydropyrimidin-1 (2H)-yl)-1-oxoisoindolin-5-yl)methyl)piperazin-1-yl)piperidin-1-yl)pyridin-2-yl)amino)-N,N-dimethyl-7H-pyrrolo[2,3-d]pyrimidine-6-carboxamide;
1-(5-((4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)methyl)-1-oxoisoindolin-2-yl)dihydropyrimidine-2,4 (1H,3H)-dione;
1-(5-((4-(6-((5-fluoro-4-(4-fluoro-1-isopropyl-2-methyl-1H-benzo[d]imidazol-6-yl)pyrimidin-2-yl)amino)pyridin-3-yl)piperazin-1-yl)methyl)-1-oxoisoindolin-2-yl)dihydropyrimidine-2,4 (1H,3H)-dione;
1-(5-(((1-(6-((5-fluoro-4-(4-fluoro-1-isopropyl-2-methyl-1H-benzo[d]imidazol-6-yl)pyrimidin-2-yl)amino)pyridin-3-yl)piperidin-4-yl)(methyl)amino)methyl)-1-oxoisoindolin-2-yl)dihydropyrimidine-2,4 (1H,3H)-dione;
1-(5-((4-(1-(6-((5-fluoro-4-(4-fluoro-1-isopropyl-2-methyl-1H-benzo[d]imidazol-6-yl)pyrimidin-2-yl)amino)pyridin-3-yl)piperidin-4-yl)piperazin-1-yl)methyl)-1-oxoisoindolin-2-yl)dihydropyrimidine-2,4 (1H,3H)-dione;
1-(5-((4-(6-((6-acetyl-8-cyclopentyl-5-methyl-7-oxo-7,8-dihydropyrido[2,3-d]pyrimidin-2-yl)amino)pyridin-3-yl)piperazin-1-yl)methyl)-1-oxoisoindolin-2-yl)dihydropyrimidine-2,4 (1H,3H)-dione;
1-(5-(((1-(6-((6-acetyl-8-cyclopentyl-5-methyl-7-oxo-7,8-dihydropyrido[2,3-d]pyrimidin-2-yl)amino)pyridin-3-yl)piperidin-4-yl)(methyl)amino)methyl)-1-oxoisoindolin-2-yl)dihydropyrimidine-2,4 (1H,3H)-dione;
1-(5-((4-(1-(6-((6-acetyl-8-cyclopentyl-5-methyl-7-oxo-7,8-dihydropyrido[2,3-d]pyrimidin-2-yl)amino)pyridin-3-yl)piperidin-4-yl)piperazin-1-yl)methyl)-1-oxoisoindolin-2-yl)dihydropyrimidine-2,4 (1H,3H)-dione;
1-(5-(((1-(4-((5-chloro-4-((2-(dimethylphosphoryl)phenyl)amino)pyrimidin-2-yl)amino)-3-methoxyphenyl) piperidin-4-yl)(methyl)amino)methyl)-1-oxoisoindolin-2-yl)dihydropyrimidine-2,4 (1H,3H)-dione:
1-(5-(((1-(4-((5-chloro-4-((2-(dimethylphosphoryl)phenyl)amino)pyrimidin-2-yl)amino)-3-methoxyphenyl) piperidin-4-yl)amino)methyl)-1-oxoisoindolin-2-yl)dihydropyrimidine-2,4 (1H,3H)-dione;
1-(5-((4-(4-((5-chloro-4-((2-(dimethylphosphoryl)phenyl)amino)pyrimidin-2-yl)amino)-3-methoxyphenyl) piperazin-1-yl)methyl)-1-oxoisoindolin-2-yl)dihydropyrimidine-2,4 (1H,3H)-dione;
1-(5-((4-(1-(4-((5-chloro-4-((2-(dimethylphosphoryl)phenyl)amino)pyrimidin-2-yl)amino)-3-methoxyphenyl) piperidin-4-yl)piperazin-1-yl)methyl)-1-oxoisoindolin-2-yl)dihydropyrimidine-2,4 (1H,3H)-dione;
8-(4-(4-((2-(2,4-dioxotetrahydropyrimidin-1 (2H)-yl)-1-oxoisoindolin-5-yl)methyl)piperazin-1-yl)piperidin-1-yl)-9-ethyl-6,6-dimethyl-11-oxo-6,11-dihydro-5H-benzo[b]carbazole-3-carbonitrile;
8-(4-((2-(2,4-dioxotetrahydropyrimidin-1 (2H)-yl)-1-oxoisoindolin-5-yl)methyl)piperazin-1-yl)-9-ethyl-6,6-dimethyl-11-oxo-6,11-dihydro-5H-benzo[b]carbazole-3-carbonitrile;
8-(4-(((2-(2,4-dioxotetrahydropyrimidin-1 (2H)-yl)-1-oxoisoindolin-5-yl)methyl)(methyl)amino) piperidin-1-yl)-9-ethyl-6,6-dimethyl-11-oxo-6,11-dihydro-5H-benzo[b]carbazole-3-carbonitrile;
2-((2-((4-(4-(4-((2-(2,4-dioxotetrahydropyrimidin-1 (2H)-yl)-1-oxoisoindolin-5-yl)methyl)piperazin-1-yl) piperidin-1-yl)-2-methoxyphenyl)amino)-5-(trifluoromethyl)pyridin-4-yl)amino)-N-methylbenzamide;
2-((2-((4-(4-(((2-(2,4-dioxotetrahydropyrimidin-1 (2H)-yl)-1-oxoisoindolin-5-yl)methyl)amino) piperidin-1-yl)-2-methoxyphenyl)amino)-5-(trifluoromethyl)pyridin-4-yl)amino)-N-methylbenzamide;
2-((2-((4-(4-(((2-(2,4-dioxotetrahydropyrimidin-1 (2H)-yl)-1-oxoisoindolin-5-yl)methyl)(methyl)amino) piperidin-1-yl)-2-methoxyphenyl)amino)-5-(trifluoromethyl)pyridin-4-yl)amino)-N-methylbenzamide;
2-((2-((4-(4-((2-(2,4-dioxotetrahydropyrimidin-1 (2H)-yl)-1-oxoisoindolin-5-yl)methyl)piperazin-1-yl)-2-methoxyphenyl)amino)-5-(trifluoromethyl)pyridin-4-yl)amino)-N-methylbenzamide;
2-((2-((4-(1-((2-(2,4-dioxotetrahydropyrimidin-1 (2H)-yl)-1-oxoisoindolin-5-yl)methyl)piperidin-4-yl)-2-isopropoxy-5-methylphenyl)amino)-5-(trifluoromethyl)pyridin-4-yl)amino)-N-methylbenzamide;
2-((2-((4-(4-((2-(2,4-dioxotetrahydropyrimidin-1 (2H)-yl)-1-oxoisoindolin-5-yl)methyl)piperazin-1-yl)-2-isopropoxy-5-methylphenyl)amino)-5-(trifluoromethyl)pyridin-4-yl)amino)-N-methylbenzamide;
2-((2-((4-(4-(((2-(2,4-dioxotetrahydropyrimidin-1 (2H)-yl)-1-oxoisoindolin-5-yl)methyl)(methyl)amino) piperidin-1-yl)-2-isopropoxy-5-methylphenyl)amino)-5-(trifluoromethyl)pyridin-4-yl)amino)-N-methylbenzamide;
2-((2-((4-(4-(1-((2-(2,4-dioxotetrahydropyrimidin-1 (2H)-yl)-1-oxoisoindolin-5-yl)methyl)piperidin-4-yl) piperazin-1-yl)-2-isopropoxy-5-methylphenyl)amino)-5-(trifluoromethyl)pyridin-4-yl)amino)-N-methylbenzamide;
N-(1-((2-(2,4-dioxotetrahydropyrimidin-1 (2H)-yl)-1-oxoisoindolin-5-yl)methyl)piperidin-4-yl)-2-fluoro-5-methoxy-4-((4-((2-methyl-3-oxoisoindolin-4-yl)oxy)-5-(trifluoromethyl)pyrimidin-2-yl)amino)benzamide;
N-(3-(((2-((4-(4-((2-(2,4-dioxotetrahydropyrimidin-1 (2H)-yl)-1-oxoisoindolin-5-yl)methyl)piperazin-1-yl)phenyl)amino)-5-(trifluoromethyl)pyrimidin-4-yl)amino)methyl)pyrazin-2-yl)-N-methylmethanesulfonamide;
N-(3-(((2-((4-(4-(((2-(2,4-dioxotetrahydropyrimidin-1 (2H)-yl)-3-oxoisoindolin-5-yl)methyl)(methyl)amino) piperidin-1-yl)phenyl)amino)-5-(trifluoromethyl)pyrimidin-4-yl)amino)methyl)pyrazin-2-yl)-N-methylmethanesulfonamide;
N-(3-(((2-((4-(4-(4-((2-(2,4-dioxotetrahydropyrimidin-1 (2H)-yl)-1-oxoisoindolin-5-yl)methyl)piperazin-1-yl) piperidin-1-yl)phenyl)amino)-5-(trifluoromethyl)pyrimidin-4-yl)amino)methyl)pyrazin-2-yl)-N-methylmethanesulfonamide;
N-(3-(((2-((4-(4-((2-(2,4-dioxotetrahydropyrimidin-1 (2H)-yl)-1-oxoisoindolin-5-yl)methyl)piperazin-1-yl)phenyl)amino)-5-(trifluoromethyl)pyrimidin-4-yl)amino)methyl)phenyl)-N-methylmethanesulfonamide;
N-(3-(((2-((4-(1-((2-(2,4-dioxotetrahydropyrimidin-1 (2H)-yl)-1-oxoisoindolin-5-yl)methyl)piperidin-4-yl)phenyl)amino)-5-(trifluoromethyl)pyrimidin-4-yl)amino)methyl)phenyl)-N-methylmethanesulfonamide;
N-(3-(((2-((4-(4-(((2-(2,4-dioxotetrahydropyrimidin-1 (2H)-yl)-1-oxoisoindolin-5-yl)methyl)(methyl)amino) piperidin-1-yl)phenyl)amino)-5-(trifluoromethyl)pyrimidin-4-yl)amino)methyl)phenyl)-N-methylmethanesulfonamide;
N-(3-(((2-((4-(4-(4-((2-(2,4-dioxotetrahydropyrimidin-1 (2H)-yl)-1-oxoisoindolin-5-yl)methyl)piperazin-1-yl) piperidin-1-yl)phenyl)amino)-5-(trifluoromethyl)pyrimidin-4-yl)amino)methyl)phenyl)-N-methylmethanesulfonamide;
1-(5-((4-(4-((4-((3-(methylsulfonyl)benzyl)amino)-5-(trifluoromethyl)pyrimidin-2-yl)amino)phenyl) piperazin-1-yl)methyl)-1-oxoisoindolin-2-yl)dihydropyrimidine-2,4 (1H,3H)-dione;
2-(2-((cyclopropylmethyl)amino)pyridin-4-yl)-N-(3-(difluoromethyl)-1-(1-((2-(2,4-dioxotetrahydropyrimidin-1 (2H)-yl)-1-oxoisoindolin-5-yl)methyl)piperidin-4-yl)-1H-pyrazol-4-yl) oxazole-4-carboxamide;
2-(2-((cyclopropylmethyl)amino)pyridin-4-yl)-N-(3-(difluoromethyl)-1-(1-((1-(2-(2,4-dioxotetrahydropyrimidin-1 (2H)-yl)-1-oxoisoindolin-5-yl)piperidin-4-yl)methyl)piperidin-4-yl)-1H-pyrazol-4-yl) oxazole-4-carboxamide;
2-(2-((cyclopropylmethyl)amino)pyridin-4-yl)-N-(3-(difluoromethyl)-1-(1-((1-(2-(2,4-dioxotetrahydropyrimidin-1 (2H)-yl)-1-oxoisoindolin-4-yl)piperidin-4-yl)methyl)piperidin-4-yl)-1H-pyrazol-4-yl) oxazole-4-carboxamide;
2-(2-((cyclopropylmethyl)amino)pyridin-4-yl)-N-(3-(difluoromethyl)-1-(1-((1′-(2-(2,4-dioxotetrahydropyrimidin-1 (2H)-yl)-1-oxoisoindolin-5-yl)-[1,4′-bipiperidin]-4-yl)methyl)piperidin-4-yl)-1H-pyrazol-4-yl) oxazole-4-carboxamide;
2-(2-((cyclopropylmethyl)amino)pyridin-4-yl)-N-(3-(difluoromethyl)-1-(4-(4-((2-(2,4-dioxotetrahydropyrimidin-1 (2H)-yl)-1-oxoisoindolin-5-yl)methyl)piperazin-1-yl)phenyl)-1H-pyrazol-4-yl) oxazole-4-carboxamide;
N-(2-(1-((2-(2,4-dioxotetrahydropyrimidin-1 (2H)-yl)-1-oxoisoindolin-5-yl)methyl)piperidin-4-yl)-6-methoxy-2H-indazol-5-yl)-3-(trifluoromethyl)benzamide;
N-(2-(1-((1-(2-(2,4-dioxotetrahydropyrimidin-1 (2H)-yl)-1-oxoisoindolin-5-yl)piperidin-4-yl)methyl)piperidin-4-yl)-6-methoxy-2H-indazol-5-yl)-3-(trifluoromethyl)benzamide;
N-(2-(1-((2-(2,4-dioxotetrahydropyrimidin-1 (2H)-yl)-1-oxoisoindolin-5-yl)methyl)piperidin-4-yl)-6-methoxy-2H-indazol-5-yl)-6-(trifluoromethyl)picolinamide;
N-(2-(1-((1-(2-(2,4-dioxotetrahydropyrimidin-1 (2H)-yl)-1-oxoisoindolin-5-yl)piperidin-4-yl)methyl)piperidin-4-yl)-6-methoxy-2H-indazol-5-yl)-6-(trifluoromethyl)picolinamide;
N-(2-(1-((2-(2,4-dioxotetrahydropyrimidin-1 (2H)-yl)-1-oxoisoindolin-5-yl)methyl)piperidin-4-yl)-6-methoxy-2H-indazol-7-yl)-3-(trifluoromethyl)benzamide;
N-(3-(difluoromethyl)-1-(1-((2-(2,4-dioxotetrahydropyrimidin-1 (2H)-yl)-1-oxoisoindolin-5-yl)methyl)piperidin-4-yl)-1H-pyrazol-4-yl)-5-morpholinopyrazolo[1,5-a]pyrimidine-3-carboxamide;
5-((1R,4R)-2-oxa-5-azabicyclo[2.2.1]heptan-5-yl)-N-(3-(difluoromethyl)-1-(1-((2-(2,4-dioxotetrahydropyrimidin-1 (2H)-yl)-1-oxoisoindolin-5-yl)methyl)piperidin-4-yl)-1H-pyrazol-4-yl)pyrazolo[1,5-alpyrimidine-3-carboxamide;
5-((1R,4R)-2-oxa-5-azabicyclo[2.2.1]heptan-5-yl)-N-(3-(difluoromethyl)-1-(1-((1-(2-(2,4-dioxotetrahydropyrimidin-1 (2H)-yl)-1-oxoisoindolin-5-yl)piperidin-4-yl)methyl)piperidin-4-yl)-1H-pyrazol-4-yl)pyrazolo[1,5-a]pyrimidine-3-carboxamide;
5-((1R,4R)-2-oxa-5-azabicyclo[2.2.1]heptan-5-yl)-N-(3-(difluoromethyl)-1-((1r,4R)-4-((4-(1-(2-(2,4-dioxotetrahydropyrimidin-1 (2H)-yl)-1-oxoisoindolin-5-yl)piperidin-4-yl)piperazin-1-yl)methyl)cyclohexyl)-1H-pyrazol-4-yl)pyrazolo[1,5-a]pyrimidine-3-carboxamide;
5-((1R,4R)-2-oxa-5-azabicyclo[2.2.1]heptan-5-yl)-N-(3-(difluoromethyl)-1-((1r,4R)-4-((4-(1-(2-(2,4-dioxotetrahydropyrimidin-1 (2H)-yl)-1-oxoisoindolin-5-yl)-3,3-difluoropiperidin-4-yl)piperazin-1-yl)methyl)cyclohexyl)-1H-pyrazol-4-yl)pyrazolo[1,5-a]pyrimidine-3-carboxamide;
N-(5-((4-((2-(2,4-dioxotetrahydropyrimidin-1 (2H)-yl)-1-oxoisoindolin-5-yl)methyl)piperazin-1-yl)methyl)-1-((1s,4s)-4-(hydroxymethyl)cyclohexyl)-1H-benzo[d]imidazol-2-yl)-3-(trifluoromethyl)benzamide;
N-(5-(4-((2-(2,4-dioxotetrahydropyrimidin-1 (2H)-yl)-1-oxoisoindolin-5-yl)methyl)piperazin-1-yl)-1-((1s,4s)-4-(hydroxymethyl)cyclohexyl)-1H-benzo[d]imidazol-2-yl)-3-(trifluoromethyl)benzamide;
N-(5-(4-((1-(2-(2,4-dioxotetrahydropyrimidin-1 (2H)-yl)-1-oxoisoindolin-5-yl)piperidin-4-yl)methyl)piperazin-1-yl)-1-((1s,4s)-4-(hydroxymethyl)cyclohexyl)-1H-benzo[d]imidazol-2-yl)-3-(trifluoromethyl)benzamide;
N-(5-(4-(1-((2-(2,4-dioxotetrahydropyrimidin-1 (2H)-yl)-1-oxoisoindolin-5-yl)methyl)piperidin-4-yl) piperazin-1-yl)-1-((1s,4s)-4-(hydroxymethyl)cyclohexyl)-1H-benzo[d]imidazol-2-yl)-3-(trifluoromethyl)benzamide;
6-(5-cyano-1H-pyrrolo[2,3-b]pyridin-1-yl)-N-(1-((2-(2,4-dioxotetrahydropyrimidin-1 (2H)-yl)-1-oxoisoindolin-5-yl)methyl)piperidin-4-yl)-4-(isopropylamino) nicotinamide;
N-(3-(difluoromethyl)-1-(1-((2-(2,4-dioxotetrahydropyrimidin-1 (2H)-yl)-1-oxoisoindolin-5-yl)methyl)piperidin-4-yl)-1H-pyrazol-4-yl)-2-(2-((2,2,2-trifluoroethyl)amino)pyridin-4-yl) oxazole-4-carboxamide;
N-(3-(difluoromethyl)-1-(1-((1-(2-(2,4-dioxotetrahydropyrimidin-1 (2H)-yl)-1-oxoisoindolin-5-yl)piperidin-4-yl)methyl)piperidin-4-yl)-1H-pyrazol-4-yl)-2-(2-((2,2,2-trifluoroethyl)amino)pyridin-4-yl) oxazole-4-carboxamide;
N-(3-(difluoromethyl)-1-(1-((1′-(2-(2,4-dioxotetrahydropyrimidin-1 (2H)-yl)-1-oxoisoindolin-5-yl)-[1,4′-bipiperidin]-4-yl)methyl)piperidin-4-yl)-1H-pyrazol-4-yl)-2-(2-((2,2,2-trifluoroethyl)amino)pyridin-4-yl) oxazole-4-carboxamide;
N-(3-(difluoromethyl)-1-(4-(4-((2-(2,4-dioxotetrahydropyrimidin-1 (2H)-yl)-1-oxoisoindolin-5-yl)methyl)piperazin-1-yl)phenyl)-1H-pyrazol-4-yl)-2-(2-((2,2,2-trifluoroethyl)amino)pyridin-4-yl) oxazole-4-carboxamide;
(R)-N-(2-(4-((2-(2,4-dioxotetrahydropyrimidin-1 (2H)-yl)-1-oxoisoindolin-5-yl)methyl)piperazin-1-yl)-5-(3-hydroxypyrrolidin-1-yl) oxazolo[4,5-b]pyridin-6-yl)-2-(2-methylpyridin-4-yl) oxazole-4-carboxamide;
N-(2-(1-((2-(2,4-dioxotetrahydropyrimidin-1 (2H)-yl)-1-oxoisoindolin-5-yl)methyl)piperidin-4-yl)-5-(2-hydroxypropan-2-yl)benzo[d]thiazol-6-yl)-6-(trifluoromethyl)picolinamide;
N-(2-(1-((2-(2,4-dioxotetrahydropyrimidin-1 (2H)-yl)-1-oxoisoindolin-5-yl)methyl)piperidin-4-yl)-5-(2-hydroxypropan-2-yl)benzo[d]oxazol-6-yl)-6-(trifluoromethyl)picolinamide;
N-(2-(1-((2-(2,4-dioxotetrahydropyrimidin-1 (2H)-yl)-1-oxoisoindolin-5-yl)methyl)piperidin-4-yl)-6-(2-hydroxypropan-2-yl)-2H-indazol-5-yl)-6-(trifluoromethyl)picolinamide;
6-(6-(4-((2-(2,4-dioxotetrahydropyrimidin-1 (2H)-yl)-1-oxoisoindolin-5-yl)methyl)piperazin-1-yl)pyridin-3-yl)-1-isopropyl-N-((6-methyl-2-oxo-4-propyl-1,2-dihydropyridin-3-yl)methyl)-1H-indazole-4-carboxamide;
6-(6-(4-(((2-(2,4-dioxotetrahydropyrimidin-1 (2H)-yl)-1-oxoisoindolin-5-yl)methyl)(methyl)amino) piperidin-1-yl)pyridin-3-yl)-1-isopropyl-N-((6-methyl-2-oxo-4-propyl-1,2-dihydropyridin-3-yl)methyl)-1H-indazole-4-carboxamide;
6-(6-(4-(1-((2-(2,4-dioxotetrahydropyrimidin-1 (2H)-yl)-1-oxoisoindolin-5-yl)methyl)piperidin-4-yl)piperazin-1-yl)pyridin-3-yl)-1-isopropyl-N-((6-methyl-2-oxo-4-propyl-1,2-dihydropyridin-3-yl)methyl)-1H-indazole-4-carboxamide;
N-((4,6-dimethyl-2-oxo-1,2-dihydropyridin-3-yl)methyl)-4′-((4-((2-(2,4-dioxotetrahydropyrimidin-1 (2H)-yl)-1-oxoisoindolin-5-yl)methyl)piperazin-1-yl)methyl)-5-(ethyl(tetrahydro-2H-pyran-4-yl)amino)-4-methyl-[1,1′-biphenyl]-3-carboxamide;
N-((4,6-dimethyl-2-oxo-1,2-dihydropyridin-3-yl)methyl)-4′-(4-((2-(2,4-dioxotetrahydropyrimidin-1 (2H)-yl)-1-oxoisoindolin-5-yl)methyl)piperazin-1-yl)-5-(ethyl(tetrahydro-2H-pyran-4-yl)amino)-4-methyl-[1,1′-biphenyl]-3-carboxamide;
N-((4,6-dimethyl-2-oxo-1,2-dihydropyridin-3-yl)methyl)-4′-(4-(((2-(2,4-dioxotetrahydropyrimidin-1 (2H)-yl)-1-oxoisoindolin-5-yl)methyl)(methyl)amino) piperidin-1-yl)-5-(ethyl(tetrahydro-2H-pyran-4-yl)amino)-4-methyl-[1,1′-biphenyl]-3-carboxamide;
N-((4,6-dimethyl-2-oxo-1,2-dihydropyridin-3-yl)methyl)-4′-(4-(1-((2-(2,4-dioxotetrahydropyrimidin-1 (2H)-yl)-1-oxoisoindolin-5-yl)methyl)piperidin-4-yl)piperazin-1-yl)-5-(ethyl(tetrahydro-2H-pyran-4-yl)amino)-4-methyl-[1,1′-biphenyl]-3-carboxamide;
(R)-4-(4-((4′-chloro-4,4-dimethyl-3,4,5,6-tetrahydro-[1,1′-biphenyl]-2-yl)methyl)piperazin-1-yl)-N-((4-((4-(4-((2-(2,4-dioxotetrahydropyrimidin-1 (2H)-yl)-1-oxoisoindolin-5-yl)methyl)piperazin-1-yl-2,2,3,3,5,5,6,6-d8)-1-(phenylthio)butan-2-yl)amino)-3-((trifluoromethyl)sulfonyl)phenyl)sulfonyl)benzamide;
4-(4-((4′-chloro-4,4-dimethyl-3,4,5,6-tetrahydro-[1,1′-biphenyl]-2-yl)methyl)piperazin-1-yl)-N-((4-(((2R)-4-(4-((2-(2,4-dioxotetrahydropyrimidin-1 (2H)-yl)-1-oxoisoindolin-5-yl)methyl)-3,5-dimethylpiperazin-1-yl)-1-(phenylthio)butan-2-yl)amino)-3-((trifluoromethyl)sulfonyl)phenyl)sulfonyl)benzamide;
4-(4-((4′-chloro-4,4-dimethyl-3,4,5,6-tetrahydro-[1,1′-biphenyl]-2-yl)methyl)piperazin-1-yl)-N-((4-(((2R)-4-(4-((2-(2,4-dioxotetrahydropyrimidin-1 (2H)-yl)-1-oxoisoindolin-5-yl)methyl)-2,6-dimethylpiperazin-1-yl)-1-(phenylthio)butan-2-yl)amino)-3-((trifluoromethyl)sulfonyl)phenyl)sulfonyl)benzamide;
4-(4-((4′-chloro-4,4-dimethyl-3,4,5,6-tetrahydro-[1,1′-biphenyl]-2-yl)methyl)piperazin-1-yl)-N-((4-(((2R)-4-(6-((2-(2,4-dioxotetrahydropyrimidin-1 (2H)-yl)-1-oxoisoindolin-5-yl)methyl)-3,6-diazabicyclo[3.1.1]heptan-3-yl)-1-(phenylthio)butan-2-yl)amino)-3-((trifluoromethyl)sulfonyl)phenyl)sulfonyl)benzamide;
4-(4-((4′-chloro-4,4-dimethyl-3,4,5,6-tetrahydro-[1,1′-biphenyl]-2-yl)methyl)piperazin-1-yl)-N-((4-(((2R)-4-(3-((2-(2,4-dioxotetrahydropyrimidin-1 (2H)-yl)-1-oxoisoindolin-5-yl)methyl)-3,6-diazabicyclo[3.1.1]heptan-6-yl)-1-(phenylthio)butan-2-yl)amino)-3-((trifluoromethyl)sulfonyl)phenyl)sulfonyl)benzamide;
4-(4-((4′-chloro-4,4-dimethyl-3,4,5,6-tetrahydro-[1,1′-biphenyl]-2-yl)methyl)piperazin-1-yl)-N-((4-(((2R)-4-(8-((2-(2,4-dioxotetrahydropyrimidin-1 (2H)-yl)-1-oxoisoindolin-5-yl)methyl)-3,8-diazabicyclo[3.2.1]octan-3-yl)-1-(phenylthio)butan-2-yl)amino)-3-((trifluoromethyl)sulfonyl)phenyl)sulfonyl)benzamide;
4-(4-((4′-chloro-4,4-dimethyl-3,4,5,6-tetrahydro-[1,1′-biphenyl]-2-yl)methyl)piperazin-1-yl)-N-((4-(((2R)-4-(3-((2-(2,4-dioxotetrahydropyrimidin-1 (2H)-yl)-1-oxoisoindolin-5-yl)methyl)-3,8-diazabicyclo[3.2.1]octan-8-yl)-1-(phenylthio)butan-2-yl)amino)-3-((trifluoromethyl)sulfonyl)phenyl)sulfonyl)benzamide;
4-(4-((4′-chloro-4,4-dimethyl-3,4,5,6-tetrahydro-[1,1′-biphenyl]-2-yl)methyl)piperazin-1-yl)-N-((4-(((R)-4-((1R,4R)-5-((2-(2,4-dioxotetrahydropyrimidin-1 (2H)-yl)-1-oxoisoindolin-5-yl)methyl)-2,5-diazabicyclo[2.2.1]heptan-2-yl)-1-(phenylthio)butan-2-yl)amino)-3-((trifluoromethyl)sulfonyl)phenyl)sulfonyl)benzamide;
4-(4-((4′-chloro-4,4-dimethyl-3,4,5,6-tetrahydro-[1,1′-biphenyl]-2-yl)methyl)piperazin-1-yl)-N-((4-(((R)-4-((1S,4S)-5-((2-(2,4-dioxotetrahydropyrimidin-1 (2H)-yl)-1-oxoisoindolin-5-yl)methyl)-2,5-diazabicyclo[2.2.1]heptan-2-yl)-1-(phenylthio)butan-2-yl)amino)-3-((trifluoromethyl)sulfonyl)phenyl)sulfonyl)benzamide;
4-(4-((4′-chloro-4,4-dimethyl-3,4,5,6-tetrahydro-[1,1′-biphenyl]-2-yl)methyl)piperazin-1-yl)-N-((4-(((2R)-4-(5-((2-(2,4-dioxotetrahydropyrimidin-1 (2H)-yl)-1-oxoisoindolin-5-yl)methyl)-2,5-diazabicyclo[2.2.2]octan-2-yl)-1-(phenylthio)butan-2-yl)amino)-3-((trifluoromethyl)sulfonyl)phenyl)sulfonyl)benzamide;
(R)-4-(4-((4′-chloro-4,4-dimethyl-3,4,5,6-tetrahydro-[1,1′-biphenyl]-2-yl)methyl)piperazin-1-yl)-N-((4-((4-(4-((2-(2,4-dioxotetrahydropyrimidin-1 (2H)-yl)-1-oxoisoindolin-5-yl)methyl)piperazin-1-yl)-1-(phenylthio)butan-2-yl)amino)-3-((trifluoromethyl)sulfonyl)phenyl)sulfonyl)benzamide;
2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)-N-((4-(((1-((2-(2,4-dioxotetrahydropyrimidin-1 (2H)-yl)-1-oxoisoindolin-5-yl)methyl)piperidin-4-yl)methyl)amino)-3-nitrophenyl)sulfonyl)benzamide;
(R)-N-(1-(4-(((2-(2,4-dioxotetrahydropyrimidin-1 (2H)-yl)-1-oxoisoindolin-5-yl)methyl)amino)benzyl)-1H-pyrazol-4-yl)-9-methyl-6-oxo-6,7,8,9-tetrahydropyrido[3′,2′: 4,5]pyrrolo[1,2-a]pyrazine-2-carboxamide;
(R)-N-(1-(4-(4-((2-(2,4-dioxotetrahydropyrimidin-1 (2H)-yl)-1-oxoisoindolin-5-yl)methyl)piperazin-1-yl)benzyl)-1H-pyrazol-4-yl)-9-methyl-6-oxo-6,7,8,9-tetrahydropyrido[3′,2′: 4,5]pyrrolo[1,2-a]pyrazine-2-carboxamide;
(R)-N-(1-(4-(4-(((2-(2,4-dioxotetrahydropyrimidin-1 (2H)-yl)-1-oxoisoindolin-5-yl)methyl)(methyl)amino) piperidin-1-yl)benzyl)-1H-pyrazol-4-yl)-9-methyl-6-oxo-6,7,8,9-tetrahydropyrido[3′,2′: 4,5]pyrrolo[1,2-a]pyrazine-2-carboxamide;
(R)-N-(1-(4-(4-(((2-(2,4-dioxotetrahydropyrimidin-1 (2H)-yl)-4-fluoro-1-oxoisoindolin-5-yl)methyl)(methyl)amino) piperidin-1-yl)benzyl)-1H-pyrazol-4-yl)-9-methyl-6-oxo-6,7,8,9-tetrahydropyrido[3′,2′: 4,5]pyrrolo[1,2-a]pyrazine-2-carboxamide;
(R)-N-(1-(4-(4-(4-((2-(2,4-dioxotetrahydropyrimidin-1 (2H)-yl)-1-oxoisoindolin-5-yl)methyl)piperazin-1-yl) piperidin-1-yl)benzyl)-1H-pyrazol-4-yl)-9-methyl-6-oxo-6,7,8,9-tetrahydropyrido[3′,2′: 4,5]pyrrolo[1,2-alpyrazine-2-carboxamide;
1-(5-((4-(3-amino-6-(2-hydroxyphenyl)pyridazin-4-yl)piperazin-1-yl)methyl)-1-oxoisoindolin-2-yl)dihydropyrimidine-2,4 (1H,3H)-dione;
1-(5-((4-(3-amino-6-(5-fluoro-2-hydroxyphenyl)pyridazin-4-yl)piperazin-1-yl)methyl)-1-oxoisoindolin-2-yl)dihydropyrimidine-2,4 (1H,3H)-dione;
5-((4-(4-amino-3-(4-phenoxyphenyl)-1H-pyrazolo[3,4-d]pyrimidin-1-yl)piperidin-1-yl)methyl)-2-(2,4-dioxotetrahydropyrimidin-1 (2H)-yl)isoindoline-1,3-dione;
5-((4-(4-amino-3-(4-phenoxyphenyl)-1H-pyrazolo[3,4-d]pyrimidin-1-yl)piperidin-1-yl)methyl)-2-(2,4-dioxotetrahydropyrimidin-1 (2H)-yl)isoindoline-1,3-dione;
5-(4-((4-(4-amino-3-(4-phenoxyphenyl)-1H-pyrazolo[3,4-d]pyrimidin-1-yl)piperidin-1-yl)methyl)piperidin-1-yl)-2-(2,4-dioxotetrahydropyrimidin-1 (2H)-yl)isoindoline-1,3-dione;
4-(4-((4-(4-amino-3-(4-phenoxyphenyl)-1H-pyrazolo[3,4-d]pyrimidin-1-yl)piperidin-1-yl)methyl)piperidin-1-yl)-2-(2,4-dioxotetrahydropyrimidin-1 (2H)-yl)isoindoline-1,3-dione;
5-(4-((4-(4-amino-3-(4-phenoxyphenyl)-1H-pyrazolo[3,4-d]pyrimidin-1-yl)piperidin-1-yl)methyl)-[1,4′-bipiperidin]-1′-yl)-2-(2,4-dioxotetrahydropyrimidin-1 (2H)-yl)isoindoline-1,3-dione;
2-(2,4-dioxotetrahydropyrimidin-1 (2H)-yl)-5-((4-(4-((1R,2S)-6-hydroxy-2-phenyl-1,2,3,4-tetrahydronaphthalen-1-yl)phenyl) piperazin-1-yl)methyl)isoindoline-1,3-dione;
2-(2,4-dioxotetrahydropyrimidin-1 (2H)-yl)-5-((4-(4-((3S,4R)-7-hydroxy-3-phenylchroman-4-yl)phenyl) piperazin-1-yl)methyl)isoindoline-1,3-dione;
N-((1r,3r)-3-(3-chloro-4-cyanophenoxy)-2,2,4,4-tetramethylcyclobutyl)-6-(4-((4-(2-(2,4-dioxotetrahydropyrimidin-1 (2H)-yl)-1,3-dioxoisoindolin-5-yl)piperazin-1-yl)methyl)piperidin-1-yl)nicotinamide;
N-((1r,3S)-3-(3-chloro-4-cyanophenoxy)-2,2,4,4-tetramethylcyclobutyl)-6-(4-(((1S,4S)-5-(2-(2,4-dioxotetrahydropyrimidin-1 (2H)-yl)-1,3-dioxoisoindolin-5-yl)-2,5-diazabicyclo[2.2.1]heptan-2-yl)methyl)piperidin-1-yl)nicotinamide;
N-((1r,3R)-3-(3-chloro-4-cyanophenoxy)-2,2,4,4-tetramethylcyclobutyl)-6-(4-(((1R,4R)-5-(2-(2,4-dioxotetrahydropyrimidin-1 (2H)-yl)-1,3-dioxoisoindolin-5-yl)-2,5-diazabicyclo[2.2.1]heptan-2-yl)methyl)piperidin-1-yl)nicotinamide;
N-((1r,3r)-3-(3-chloro-4-cyanophenoxy)-2,2,4,4-tetramethylcyclobutyl)-6-(4-((4-(2-(2,4-dioxotetrahydropyrimidin-1 (2H)-yl)-6-fluoro-1,3-dioxoisoindolin-5-yl)piperazin-1-yl)methyl)piperidin-1-yl)nicotinamide;
N-((1r,3r)-3-(3-chloro-4-cyanophenoxy)-2,2,4,4-tetramethylcyclobutyl)-6-(4-((8-(2-(2,4-dioxotetrahydropyrimidin-1 (2H)-yl)-1,3-dioxoisoindolin-5-yl)-3,8-diazabicyclo[3.2.1]octan-3-yl)methyl)piperidin-1-yl)nicotinamide;
N-((1r,3r)-3-(3-chloro-4-cyanophenoxy)-2,2,4,4-tetramethylcyclobutyl)-6-(4-((6-(2-(2,4-dioxotetrahydropyrimidin-1 (2H)-yl)-1,3-dioxoisoindolin-5-yl)-3,6-diazabicyclo[3.1.1]heptan-3-yl)methyl)piperidin-1-yl)nicotinamide;
N-((1r,3r)-3-(3-chloro-4-cyanophenoxy)-2,2,4,4-tetramethylcyclobutyl)-6-(4-((4-(2-(2,4-dioxotetrahydropyrimidin-1 (2H)-yl)-1,3-dioxoisoindolin-4-yl)piperazin-1-yl)methyl)piperidin-1-yl) nicotinamide;
N-((1r,3r)-3-(3-chloro-4-cyanophenoxy)-2,2,4,4-tetramethylcyclobutyl)-6-(4-((4-(2-(2,4-dioxotetrahydropyrimidin-1 (2H)-yl)-7-fluoro-1,3-dioxoisoindolin-5-yl)piperazin-1-yl)methyl)piperidin-1-yl) nicotinamide;
N-((1r,3r)-3-(3-chloro-4-cyanophenoxy)-2,2,4,4-tetramethylcyclobutyl)-6-(4-((4-(1-(2-(2,4-dioxotetrahydropyrimidin-1 (2H)-yl)-1,3-dioxoisoindolin-5-yl)-3,3-difluoropiperidin-4-yl)piperazin-1-yl)methyl)piperidin-1-yl) nicotinamide;
N-((1r,4r)-4-(3-chloro-4-cyanophenoxy)cyclohexyl)-6-(4-((4-(2-(2,4-dioxotetrahydropyrimidin-1 (2H)-yl)-1,3-dioxoisoindolin-5-yl)piperazin-1-yl)methyl)piperidin-1-yl)pyridazine-3-carboxamide;
N-((1r,4r)-4-(3-chloro-4-cyanophenoxy)cyclohexyl)-6-(4-(((1S,4S)-5-(2-(2,4-dioxotetrahydropyrimidin-1 (2H)-yl)-1,3-dioxoisoindolin-5-yl)-2,5-diazabicyclo[2.2.1]heptan-2-yl)methyl)piperidin-1-yl)pyridazine-3-carboxamide;
N-((1r,4r)-4-(3-chloro-4-cyanophenoxy)cyclohexyl)-6-(4-(((1R,4R)-5-(2-(2,4-dioxotetrahydropyrimidin-1 (2H)-yl)-1,3-dioxoisoindolin-5-yl)-2,5-diazabicyclo[2.2.1]heptan-2-yl)methyl)piperidin-1-yl)pyridazine-3-carboxamide;
N-((1r,4r)-4-(3-chloro-4-cyanophenoxy)cyclohexyl)-6-(4-((4-(2-(2,4-dioxotetrahydropyrimidin-1 (2H)-yl)-6-fluoro-1,3-dioxoisoindolin-5-yl)piperazin-1-yl)methyl)piperidin-1-yl)pyridazine-3-carboxamide;
N-((1r,4r)-4-(3-chloro-4-cyanophenoxy)cyclohexyl)-6-(4-((8-(2-(2,4-dioxotetrahydropyrimidin-1 (2H)-yl)-1,3-dioxoisoindolin-5-yl)-3,8-diazabicyclo[3.2.1]octan-3-yl)methyl)piperidin-1-yl)pyridazine-3-carboxamide;
N-((1r,4r)-4-(3-chloro-4-cyanophenoxy)cyclohexyl)-6-(4-((4-(2-(2,4-dioxotetrahydropyrimidin-1 (2H)-yl)-1,3-dioxoisoindolin-4-yl)piperazin-1-yl)methyl)piperidin-1-yl)pyridazine-3-carboxamide;
N-((1r,4r)-4-(3-chloro-4-cyanophenoxy)cyclohexyl)-6-(4-((6-(2-(2,4-dioxotetrahydropyrimidin-1 (2H)-yl)-1,3-dioxoisoindolin-5-yl)-3,6-diazabicyclo[3.1.1]heptan-3-yl)methyl)piperidin-1-yl)pyridazine-3-carboxamide;
N-((1r,4r)-4-(3-chloro-4-cyanophenoxy)cyclohexyl)-6-(4-((4-(2-(2,4-dioxotetrahydropyrimidin-1 (2H)-yl)-4-fluoro-1,3-dioxoisoindolin-5-yl)piperazin-1-yl)methyl)piperidin-1-yl)pyridazine-3-carboxamide;
N-((1r,4r)-4-(3-chloro-4-cyanophenoxy)cyclohexyl)-6-(4-((4-(2-(2,4-dioxotetrahydropyrimidin-1 (2H)-yl)-7-fluoro-1,3-dioxoisoindolin-5-yl)piperazin-1-yl)methyl)piperidin-1-yl)pyridazine-3-carboxamide;
N-((1r,4r)-4-(3-chloro-4-cyanophenoxy)cyclohexyl)-6-(4-((4-(1-(2-(2,4-dioxotetrahydropyrimidin-1 (2H)-yl)-1,3-dioxoisoindolin-5-yl)-3,3-difluoropiperidin-4-yl)piperazin-1-yl)methyl)piperidin-1-yl)pyridazine-3-carboxamide;
5-((4-(4-((9-cyclopentyl-8-(phenylamino)-9H-purin-2-yl)amino)phenyl) piperazin-1-yl)methyl)-2-(2,4-dioxotetrahydropyrimidin-1 (2H)-yl)isoindoline-1,3-dione;
5-(((1-(4-((9-cyclopentyl-8-(phenylamino)-9H-purin-2-yl)amino)phenyl) piperidin-4-yl)(methyl)amino)methyl)-2-(2,4-dioxotetrahydropyrimidin-1 (2H)-yl)isoindoline-1,3-dione;
5-((4-(1-(4-((9-cyclopentyl-8-(phenylamino)-9H-purin-2-yl)amino)phenyl) piperidin-4-yl)piperazin-1-yl)methyl)-2-(2,4-dioxotetrahydropyrimidin-1 (2H)-yl)isoindoline-1,3-dione;
5-((4-(4-((5-bromo-4-((5-(dimethylphosphoryl) quinoxalin-6-yl)amino)pyrimidin-2-yl)amino)-5-methoxy-2-methylphenyl) piperazin-1-yl)methyl)-2-(2,4-dioxotetrahydropyrimidin-1 (2H)-yl)isoindoline-1,3-dione;
5-(((1-(4-((5-bromo-4-((5-(dimethylphosphoryl) quinoxalin-6-yl)amino)pyrimidin-2-yl)amino)-5-methoxy-2-methylphenyl) piperidin-4-yl)(methyl)amino)methyl)-2-(2,4-dioxotetrahydropyrimidin-1 (2H)-yl)isoindoline-1,3-dione;
5-((4-(1-(4-((5-bromo-4-((5-(dimethylphosphoryl) quinoxalin-6-yl)amino)pyrimidin-2-yl)amino)-5-methoxy-2-methylphenyl) piperidin-4-yl)piperazin-1-yl)methyl)-2-(2,4-dioxotetrahydropyrimidin-1 (2H)-yl)isoindoline-1,3-dione;
2-(7-(((2-(2,4-dioxotetrahydropyrimidin-1 (2H)-yl)-1,3-dioxoisoindolin-5-yl)methyl)amino)-1-oxoisoindolin-2-yl)-2-(5-fluoro-2-hydroxyphenyl)-N-(thiazol-2-yl)acetamide;
2-(6-(4-((2-(2,4-dioxotetrahydropyrimidin-1 (2H)-yl)-1,3-dioxoisoindolin-5-yl)methyl)piperazin-1-yl)-1-oxoisoindolin-2-yl)-2-(5-fluoro-2-hydroxyphenyl)-N-(thiazol-2-yl)acetamide;
2-(6-(4-(((2-(2,4-dioxotetrahydropyrimidin-1 (2H)-yl)-1,3-dioxoisoindolin-5-yl)methyl)(methyl)amino) piperidin-1-yl)-1-oxoisoindolin-2-yl)-2-(5-fluoro-2-hydroxyphenyl)-N-(thiazol-2-yl)acetamide;
2-(6-(4-(4-((2-(2,4-dioxotetrahydropyrimidin-1 (2H)-yl)-1,3-dioxoisoindolin-5-yl)methyl)piperazin-1-yl) piperidin-1-yl)-1-oxoisoindolin-2-yl)-2-(5-fluoro-2-hydroxyphenyl)-N-(thiazol-2-yl)acetamide;
2-(6-(4-(4-((2-(2,4-dioxotetrahydropyrimidin-1 (2H)-yl)-1,3-dioxoisoindolin-5-yl)methyl)piperazin-1-yl)phenyl)-1-oxoisoindolin-2-yl)-2-(5-fluoro-2-hydroxyphenyl)-N-(thiazol-2-yl)acetamide;
2-(6-(6-(4-((2-(2,4-dioxotetrahydropyrimidin-1 (2H)-yl)-1,3-dioxoisoindolin-5-yl)methyl)piperazin-1-yl)pyridazin-3-yl)-1-oxoisoindolin-2-yl)-2-(5-fluoro-2-hydroxyphenyl)-N-(thiazol-2-yl)acetamide;
2-(6,7-dihydro-5H-pyrrolo[1,2-c]imidazol-1-yl)-2-(6-(4-(4-((2-(2,4-dioxotetrahydropyrimidin-1 (2H)-yl)-1,3-dioxoisoindolin-5-yl)methyl)piperazin-1-yl)phenyl)-4-fluoro-1-oxoisoindolin-2-yl)-N-(thiazol-2-yl)acetamide;
2-(6,7-dihydro-5H-pyrrolo[1,2-c]imidazol-1-yl)-2-(6-(4-((1S,4S)-5-((2-(2,4-dioxotetrahydropyrimidin-1 (2H)-yl)-1,3-dioxoisoindolin-5-yl)methyl)-2,5-diazabicyclo[2.2.1]heptan-2-yl)phenyl)-4-fluoro-1-oxoisoindolin-2-yl)-N-(thiazol-2-yl)acetamide;
2-(6,7-dihydro-5H-pyrrolo[1,2-c]imidazol-1-yl)-2-(6-(4-((1R,4R)-5-((2-(2,4-dioxotetrahydropyrimidin-1 (2H)-yl)-1,3-dioxoisoindolin-5-yl)methyl)-2,5-diazabicyclo[2.2.1]heptan-2-yl)phenyl)-4-fluoro-1-oxoisoindolin-2-yl)-N-(thiazol-2-yl)acetamide;
2-(6,7-dihydro-5H-pyrrolo[1,2-c]imidazol-1-yl)-2-(6-(4-(8-((2-(2,4-dioxotetrahydropyrimidin-1 (2H)-yl)-1,3-dioxoisoindolin-5-yl)methyl)-3,8-diazabicyclo[3.2.1]octan-3-yl)phenyl)-4-fluoro-1-oxoisoindolin-2-yl)-N-(thiazol-2-yl)acetamide;
2-(6,7-dihydro-5H-pyrrolo[1,2-c]imidazol-1-yl)-2-(6-(4-(4-(((2-(2,4-dioxotetrahydropyrimidin-1 (2H)-yl)-1,3-dioxoisoindolin-5-yl)methyl)(methyl)amino) piperidin-1-yl)phenyl)-4-fluoro-1-oxoisoindolin-2-yl)-N-(thiazol-2-yl)acetamide;
2-(6,7-dihydro-5H-pyrrolo[1,2-c]imidazol-1-yl)-2-(6-(4-(4-(4-((2-(2,4-dioxotetrahydropyrimidin-1 (2H)-yl)-1,3-dioxoisoindolin-5-yl)methyl)piperazin-1-yl)piperidin-1-yl)phenyl)-4-fluoro-1-oxoisoindolin-2-yl)-N-(thiazol-2-yl)acetamide;
2-(6,7-dihydro-5H-pyrrolo[1,2-c]imidazol-1-yl)-2-(6-(4-(4-(4-((2-(2,4-dioxotetrahydropyrimidin-1 (2H)-yl)-1,3-dioxoisoindolin-5-yl)methyl)piperazin-1-yl)-3,3-difluoropiperidin-1-yl)phenyl)-4-fluoro-1-oxoisoindolin-2-yl)-N-(thiazol-2-yl)acetamide;
2-(6,7-dihydro-5H-pyrrolo[1,2-c]imidazol-1-yl)-2-(6-(4-(4-(1-((2-(2,4-dioxotetrahydropyrimidin-1 (2H)-yl)-1,3-dioxoisoindolin-5-yl)methyl)piperidin-4-yl)piperazin-1-yl)phenyl)-4-fluoro-1-oxoisoindolin-2-yl)-N-(thiazol-2-yl)acetamide;
2-(6,7-dihydro-5H-pyrrolo[1,2-c]imidazol-1-yl)-2-(6-(4-(4-(1-((2-(2,4-dioxotetrahydropyrimidin-1 (2H)-yl)-1,3-dioxoisoindolin-5-yl)methyl)-3,3-difluoropiperidin-4-yl)piperazin-1-yl)phenyl)-4-fluoro-1-oxoisoindolin-2-yl)-N-(thiazol-2-yl)acetamide;
5-((4-(6-((2-(1-(cyclopropylsulfonyl)-1H-pyrazol-4-yl)pyrimidin-4-yl)amino)-1-isopropyl-1H-pyrazolo[4,3-c]pyridin-3-yl)piperazin-1-yl)methyl)-2-(2,4-dioxotetrahydropyrimidin-1 (2H)-yl)isoindoline-1,3-dione;
5-(((1-(6-((2-(1-(cyclopropylsulfonyl)-1H-pyrazol-4-yl)pyrimidin-4-yl)amino)-1-isopropyl-1H-pyrazolo[4,3-c]pyridin-3-yl)piperidin-4-yl)(methyl)amino)methyl)-2-(2,4-dioxotetrahydropyrimidin-1 (2H)-yl)isoindoline-1,3-dione;
5-((4-(1-(6-((2-(1-(cyclopropylsulfonyl)-1H-pyrazol-4-yl)pyrimidin-4-yl)amino)-1-isopropyl-1H-pyrazolo[4,3-c]pyridin-3-yl)piperidin-4-yl)piperazin-1-yl)methyl)-2-(2,4-dioxotetrahydropyrimidin-1 (2H)-yl)isoindoline-1,3-dione;
2-(2,4-dioxotetrahydropyrimidin-1 (2H)-yl)-5-((4-(1-isopropyl-6-((2-(4-methoxypiperidin-1-yl)pyrimidin-4-yl)amino)-1H-pyrazolo[4,3-c]pyridin-3-yl)piperazin-1-yl)methyl)isoindoline-1,3-dione;
2-(2,4-dioxotetrahydropyrimidin-1 (2H)-yl)-5-(((1-(1-isopropyl-6-((2-(4-methoxypiperidin-1-yl)pyrimidin-4-yl)amino)-1H-pyrazolo[4,3-c]pyridin-3-yl)piperidin-4-yl)(methyl)amino)methyl)isoindoline-1,3-dione;
2-(2,4-dioxotetrahydropyrimidin-1 (2H)-yl)-5-((4-(1-(1-isopropyl-6-((2-(4-methoxypiperidin-1-yl)pyrimidin-4-yl)amino)-1H-pyrazolo[4,3-c]pyridin-3-yl)piperidin-4-yl)piperazin-1-yl)methyl)isoindoline-1,3-dione;
N-(5-(((5-(tert-butyl) oxazol-2-yl)methyl)thio) thiazol-2-yl)-1-((2-(2,4-dioxotetrahydropyrimidin-1 (2H)-yl)-1,3-dioxoisoindolin-5-yl)methyl)piperidine-4-carboxamide;
N-(5-(((5-(tert-butyl) oxazol-2-yl)methyl)thio) thiazol-2-yl)-1′-((2-(2,4-dioxotetrahydropyrimidin-1 (2H)-yl)-1,3-dioxoisoindolin-5-yl)methyl)-[1,4′-bipiperidine]-4-carboxamide;
4-(2,6-dichlorobenzamido)-N-(1-((2-(2,4-dioxotetrahydropyrimidin-1 (2H)-yl)-1,3-dioxoisoindolin-5-yl)methyl)piperidin-4-yl)-1H-pyrazole-3-carboxamide;
4-(2,6-dichlorobenzamido)-N-(1-((4-(2-(2,4-dioxotetrahydropyrimidin-1 (2H)-yl)-1,3-dioxoisoindolin-5-yl) piperazin-1-yl)methyl)piperidin-4-yl)-1H-pyrazole-3-carboxamide;
N-(5-cyclobutyl-1H-pyrazol-3-yl)-2-(4-((2-(2,4-dioxotetrahydropyrimidin-1 (2H)-yl)-1,3-dioxoisoindolin-5-yl)methoxy)phenyl)acetamide;
5-((4-((5-chloro-4-(5-(cyclopropylmethyl)-1-methyl-1H-pyrazol-4-yl)pyrimidin-2-yl)amino) piperidin-1-yl)methyl)-2-(2,4-dioxotetrahydropyrimidin-1 (2H)-yl)isoindoline-1,3-dione;
4-(((4-(5-chloro-2-((1-((2-(2,4-dioxotetrahydropyrimidin-1 (2H)-yl)-1,3-dioxoisoindolin-5-yl)methyl)piperidin-4-yl)amino)pyridin-4-yl) thiazol-2-yl)amino)methyl)tetrahydro-2H-pyran-4-carbonitrile;
4-(((5′-chloro-2′-((1-((2-(2,4-dioxotetrahydropyrimidin-1 (2H)-yl)-1,3-dioxoisoindolin-5-yl)methyl)piperidin-4-yl)amino)-[2,4′-bipyridin]-6-yl)amino)methyl)tetrahydro-2H-pyran-4-carbonitrile;
N-(4-(chlorodifluoromethoxy)phenyl)-6-(4-((2-(2,4-dioxotetrahydropyrimidin-1 (2H)-yl)-1,3-dioxoisoindolin-5-yl)methyl)piperazin-1-yl)-5-(1H-pyrazol-3-yl) nicotinamide;
N-(4-(chlorodifluoromethoxy)phenyl)-6-(4-(((2-(2,4-dioxotetrahydropyrimidin-1 (2H)-yl)-1,3-dioxoisoindolin-5-yl)methyl)(methyl)amino) piperidin-1-yl)-5-(1H-pyrazol-3-yl) nicotinamide;
N-(4-(chlorodifluoromethoxy)phenyl)-6-(4-(1-((2-(2,4-dioxotetrahydropyrimidin-1 (2H)-yl)-1,3-dioxoisoindolin-5-yl)methyl)piperidin-4-yl)piperazin-1-yl)-5-(1H-pyrazol-3-yl) nicotinamide;
N-(4-(chlorodifluoromethoxy)phenyl)-6-(4-(4-((2-(2,4-dioxotetrahydropyrimidin-1 (2H)-yl)-1,3-dioxoisoindolin-5-yl)methyl)piperazin-1-yl)piperidin-1-yl)-5-(1H-pyrazol-3-yl) nicotinamide;
N-(4-((4-((2-(2,4-dioxotetrahydropyrimidin-1 (2H)-yl)-1,3-dioxoisoindolin-5-yl)methyl)piperazin-1-yl)methyl)-3-(trifluoromethyl)phenyl)-3-(imidazo[1,2-b]pyridazin-3-ylethynyl)-4-methylbenzamide;
N-(4-(4-((2-(2,4-dioxotetrahydropyrimidin-1 (2H)-yl)-1,3-dioxoisoindolin-5-yl)methyl)piperazin-1-yl)-3-(trifluoromethyl)phenyl)-3-(imidazo[1,2-b]pyridazin-3-ylethynyl)-4-methylbenzamide;
N-(4-(4-(1-((2-(2,4-dioxotetrahydropyrimidin-1 (2H)-yl)-1,3-dioxoisoindolin-5-yl)methyl)piperidin-4-yl) piperazin-1-yl)-3-(trifluoromethyl)phenyl)-3-(imidazo[1,2-b]pyridazin-3-ylethynyl)-4-methylbenzamide;
N-(4-(4-(((2-(2,4-dioxotetrahydropyrimidin-1 (2H)-yl)-1,3-dioxoisoindolin-5-yl)methyl)(methyl)amino) piperidin-1-yl)-3-(trifluoromethyl)phenyl)-3-(imidazo[1,2-b]pyridazin-3-ylethynyl)-4-methylbenzamide;
7-cyclopentyl-2-((5-(4-((2-(2,4-dioxotetrahydropyrimidin-1 (2H)-yl)-1,3-dioxoisoindolin-5-yl)methyl)piperazin-1-yl)pyridin-2-yl)amino)-N,N-dimethyl-7H-pyrrolo[2,3-d]pyrimidine-6-carboxamide;
7-cyclopentyl-2-((5-(4-((4-(2-(2,4-dioxotetrahydropyrimidin-1 (2H)-yl)-1,3-dioxoisoindolin-5-yl)piperazin-1-yl)methyl)piperidin-1-yl)pyridin-2-yl)amino)-N,N-dimethyl-7H-pyrrolo[2,3-d]pyrimidine-6-carboxamide;
7-cyclopentyl-2-((5-(4-(((2-(2,4-dioxotetrahydropyrimidin-1 (2H)-yl)-1,3-dioxoisoindolin-5-yl)methyl)(methyl)amino) piperidin-1-yl)pyridin-2-yl)amino)-N,N-dimethyl-7H-pyrrolo[2,3-d]pyrimidine-6-carboxamide;
7-cyclopentyl-2-((5-(4-(4-((2-(2,4-dioxotetrahydropyrimidin-1 (2H)-yl)-1,3-dioxoisoindolin-5-yl)methyl)piperazin-1-yl)piperidin-1-yl)pyridin-2-yl)amino)-N,N-dimethyl-7H-pyrrolo[2,3-d]pyrimidine-6-carboxamide;
7-cyclopentyl-2-((5-(4-(((1S,4S)-5-(2-(2,4-dioxotetrahydropyrimidin-1 (2H)-yl)-1,3-dioxoisoindolin-5-yl)-2,5-diazabicyclo[2.2.1]heptan-2-yl)methyl)piperidin-1-yl)pyridin-2-yl)amino)-N,N-dimethyl-7H-pyrrolo[2,3-d]pyrimidine-6-carboxamide;
7-cyclopentyl-2-((5-(4-(((1R,4R)-5-(2-(2,4-dioxotetrahydropyrimidin-1 (2H)-yl)-1,3-dioxoisoindolin-5-yl)-2,5-diazabicyclo[2.2.1]heptan-2-yl)methyl)piperidin-1-yl)pyridin-2-yl)amino)-N,N-dimethyl-7H-pyrrolo[2,3-d]pyrimidine-6-carboxamide;
7-cyclopentyl-2-((5-(4-((4-(2-(2,4-dioxotetrahydropyrimidin-1 (2H)-yl)-6-fluoro-1,3-dioxoisoindolin-5-yl) piperazin-1-yl)methyl)piperidin-1-yl)pyridin-2-yl)amino)-N,N-dimethyl-7H-pyrrolo[2,3-d]pyrimidine-6-carboxamide;
7-cyclopentyl-2-((5-(4-((8-(2-(2,4-dioxotetrahydropyrimidin-1 (2H)-yl)-1,3-dioxoisoindolin-5-yl)-3,8-diazabicyclo[3.2.1]octan-3-yl)methyl)piperidin-1-yl)pyridin-2-yl)amino)-N,N-dimethyl-7H-pyrrolo[2,3-d]pyrimidine-6-carboxamide;
7-cyclopentyl-2-((5-(4-((6-(2-(2,4-dioxotetrahydropyrimidin-1 (2H)-yl)-1,3-dioxoisoindolin-5-yl)-3,6-diazabicyclo[3.1.1]heptan-3-yl)methyl)piperidin-1-yl)pyridin-2-yl)amino)-N,N-dimethyl-7H-pyrrolo[2,3-d]pyrimidine-6-carboxamide;
7-cyclopentyl-2-((5-(4-((4-(2-(2,4-dioxotetrahydropyrimidin-1 (2H)-yl)-1,3-dioxoisoindolin-4-yl)piperazin-1-yl)methyl)piperidin-1-yl)pyridin-2-yl)amino)-N,N-dimethyl-7H-pyrrolo[2,3-d]pyrimidine-6-carboxamide;
7-cyclopentyl-2-((5-(4-((4-(2-(2,4-dioxotetrahydropyrimidin-1 (2H)-yl)-4-fluoro-1,3-dioxoisoindolin-5-yl) piperazin-1-yl)methyl)piperidin-1-yl)pyridin-2-yl)amino)-N,N-dimethyl-7H-pyrrolo[2,3-d]pyrimidine-6-carboxamide;
7-cyclopentyl-2-((5-(4-((4-(2-(2,4-dioxotetrahydropyrimidin-1 (2H)-yl)-7-fluoro-1,3-dioxoisoindolin-5-yl) piperazin-1-yl)methyl)piperidin-1-yl)pyridin-2-yl)amino)-N,N-dimethyl-7H-pyrrolo[2,3-d]pyrimidine-6-carboxamide;
7-cyclopentyl-2-((5-(4-((4-(1-(2-(2,4-dioxotetrahydropyrimidin-1 (2H)-yl)-1,3-dioxoisoindolin-5-yl)piperidin-4-yl)piperazin-1-yl)methyl)piperidin-1-yl)pyridin-2-yl)amino)-N,N-dimethyl-7H-pyrrolo[2,3-d]pyrimidine-6-carboxamide;
7-cyclopentyl-2-((5-(4-((4-(1-(2-(2,4-dioxotetrahydropyrimidin-1 (2H)-yl)-1,3-dioxoisoindolin-5-yl)-3,3-difluoropiperidin-4-yl)piperazin-1-yl)methyl)piperidin-1-yl)pyridin-2-yl)amino)-N,N-dimethyl-7H-pyrrolo[2,3-d]pyrimidine-6-carboxamide;
2-(2,4-dioxotetrahydropyrimidin-1 (2H)-yl)-5-((4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)methyl)isoindoline-1,3-dione;
2-(2,4-dioxotetrahydropyrimidin-1 (2H)-yl)-5-((4-(6-((5-fluoro-4-(4-fluoro-1-isopropyl-2-methyl-1H-benzo[d]imidazol-6-yl)pyrimidin-2-yl)amino)pyridin-3-yl)piperazin-1-yl)methyl)isoindoline-1,3-dione;
2-(2,4-dioxotetrahydropyrimidin-1 (2H)-yl)-5-(((1-(6-((5-fluoro-4-(4-fluoro-1-isopropyl-2-methyl-1H-benzo[d]imidazol-6-yl)pyrimidin-2-yl)amino)pyridin-3-yl)piperidin-4-yl)(methyl)amino)methyl)isoindoline-1,3-dione;
2-(2,4-dioxotetrahydropyrimidin-1 (2H)-yl)-5-((4-(1-(6-((5-fluoro-4-(4-fluoro-1-isopropyl-2-methyl-1H-benzo[d]imidazol-6-yl)pyrimidin-2-yl)amino)pyridin-3-yl)piperidin-4-yl)piperazin-1-yl)methyl)isoindoline-1,3-dione;
5-((4-(6-((6-acetyl-8-cyclopentyl-5-methyl-7-oxo-7,8-dihydropyrido[2,3-d]pyrimidin-2-yl)amino)pyridin-3-yl) piperazin-1-yl)methyl)-2-(2,4-dioxotetrahydropyrimidin-1 (2H)-yl)isoindoline-1,3-dione;
5-(((1-(6-((6-acetyl-8-cyclopentyl-5-methyl-7-oxo-7,8-dihydropyrido[2,3-d]pyrimidin-2-yl)amino)pyridin-3-yl) piperidin-4-yl)(methyl)amino)methyl)-2-(2,4-dioxotetrahydropyrimidin-1 (2H)-yl)isoindoline-1,3-dione;
5-((4-(1-(6-((6-acetyl-8-cyclopentyl-5-methyl-7-oxo-7,8-dihydropyrido[2,3-d]pyrimidin-2-yl)amino)pyridin-3-yl)piperidin-4-yl)piperazin-1-yl)methyl)-2-(2,4-dioxotetrahydropyrimidin-1 (2H)-yl)isoindoline-1,3-dione;
5-((4-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)piperidin-1-yl)methyl)-2-(2,4-dioxotetrahydropyrimidin-1 (2H)-yl)isoindoline-1,3-dione;
5-(((1-(4-((5-chloro-4-((2-(dimethylphosphoryl)phenyl)amino)pyrimidin-2-yl)amino)-3-methoxyphenyl) piperidin-4-yl)(methyl)amino)methyl)-2-(2,4-dioxotetrahydropyrimidin-1 (2H)-yl)isoindoline-1,3-dione;
5-(((1-(4-((5-chloro-4-((2-(dimethylphosphoryl)phenyl)amino)pyrimidin-2-yl)amino)-3-methoxyphenyl) piperidin-4-yl)amino)methyl)-2-(2,4-dioxotetrahydropyrimidin-1 (2H)-yl)isoindoline-1,3-dione;
5-((4-(4-((5-chloro-4-((2-(dimethylphosphoryl)phenyl)amino)pyrimidin-2-yl)amino)-3-methoxyphenyl) piperazin-1-yl)methyl)-2-(2,4-dioxotetrahydropyrimidin-1 (2H)-yl)isoindoline-1,3-dione;
5-((4-(1-(4-((5-chloro-4-((2-(dimethylphosphoryl)phenyl)amino)pyrimidin-2-yl)amino)-3-methoxyphenyl) piperidin-4-yl)piperazin-1-yl)methyl)-2-(2,4-dioxotetrahydropyrimidin-1 (2H)-yl)isoindoline-1,3-dione;
8-(4-(4-((2-(2,4-dioxotetrahydropyrimidin-1 (2H)-yl)-1,3-dioxoisoindolin-5-yl)methyl)piperazin-1-yl) piperidin-1-yl)-9-ethyl-6,6-dimethyl-11-oxo-6,11-dihydro-5H-benzo[b]carbazole-3-carbonitrile;
8-(4-((2-(2,4-dioxotetrahydropyrimidin-1 (2H)-yl)-1,3-dioxoisoindolin-5-yl)methyl)piperazin-1-yl)-9-ethyl-6,6-dimethyl-11-oxo-6,11-dihydro-5H-benzo[b]carbazole-3-carbonitrile;
8-(4-(((2-(2,4-dioxotetrahydropyrimidin-1 (2H)-yl)-1,3-dioxoisoindolin-5-yl)methyl)(methyl)amino) piperidin-1-yl)-9-ethyl-6,6-dimethyl-11-oxo-6,11-dihydro-5H-benzo[b]carbazole-3-carbonitrile;
5-((4-(1-(4-(5,7-dimethoxy-4-oxo-3,4-dihydroquinazolin-2-yl)phenyl) piperidin-4-yl)piperazin-1-yl)methyl)-2-(2,4-dioxotetrahydropyrimidin-1 (2H)-yl)isoindoline-1,3-dione;
2-((2-((4-(4-(4-((2-(2,4-dioxotetrahydropyrimidin-1 (2H)-yl)-1,3-dioxoisoindolin-5-yl)methyl)piperazin-1-yl) piperidin-1-yl)-2-methoxyphenyl)amino)-5-(trifluoromethyl)pyridin-4-yl)amino)-N-methylbenzamide;
2-((2-((4-(4-(((2-(2,4-dioxotetrahydropyrimidin-1 (2H)-yl)-1,3-dioxoisoindolin-5-yl)methyl)amino) piperidin-1-yl)-2-methoxyphenyl)amino)-5-(trifluoromethyl)pyridin-4-yl)amino)-N-methylbenzamide;
2-((2-((4-(4-(((2-(2,4-dioxotetrahydropyrimidin-1 (2H)-yl)-1,3-dioxoisoindolin-5-yl)methyl)(methyl)amino) piperidin-1-yl)-2-methoxyphenyl)amino)-5-(trifluoromethyl)pyridin-4-yl)amino)-N-methylbenzamide;
2-((2-((4-(4-((2-(2,4-dioxotetrahydropyrimidin-1 (2H)-yl)-1,3-dioxoisoindolin-5-yl)methyl)piperazin-1-yl)-2-methoxyphenyl)amino)-5-(trifluoromethyl)pyridin-4-yl)amino)-N-methylbenzamide;
2-((2-((4-(1-((2-(2,4-dioxotetrahydropyrimidin-1 (2H)-yl)-1,3-dioxoisoindolin-5-yl)methyl)piperidin-4-yl)-2-isopropoxy-5-methylphenyl)amino)-5-(trifluoromethyl)pyridin-4-yl)amino)-N-methylbenzamide;
2-((2-((4-(4-((2-(2,4-dioxotetrahydropyrimidin-1 (2H)-yl)-1,3-dioxoisoindolin-5-yl)methyl)piperazin-1-yl)-2-isopropoxy-5-methylphenyl)amino)-5-(trifluoromethyl)pyridin-4-yl)amino)-N-methylbenzamide;
2-((2-((4-(4-(((2-(2,4-dioxotetrahydropyrimidin-1 (2H)-yl)-1,3-dioxoisoindolin-5-yl)methyl)(methyl)amino) piperidin-1-yl)-2-isopropoxy-5-methylphenyl)amino)-5-(trifluoromethyl)pyridin-4-yl)amino)-N-methylbenzamide;
2-((2-((4-(4-(1-((2-(2,4-dioxotetrahydropyrimidin-1 (2H)-yl)-1,3-dioxoisoindolin-5-yl)methyl)piperidin-4-yl) piperazin-1-yl)-2-isopropoxy-5-methylphenyl)amino)-5-(trifluoromethyl)pyridin-4-yl)amino)-N-methylbenzamide;
N-(1-((2-(2,4-dioxotetrahydropyrimidin-1 (2H)-yl)-1,3-dioxoisoindolin-5-yl)methyl)piperidin-4-yl)-2-fluoro-5-methoxy-4-((4-((2-methyl-3-oxoisoindolin-4-yl)oxy)-5-(trifluoromethyl)pyrimidin-2-yl)amino)benzamide;
N-(3-(((2-((4-(4-((2-(2,4-dioxotetrahydropyrimidin-1 (2H)-yl)-1,3-dioxoisoindolin-5-yl)methyl)piperazin-1-yl)phenyl)amino)-5-(trifluoromethyl)pyrimidin-4-yl)amino)methyl)pyrazin-2-yl)-N-methylmethanesulfonamide;
N-(3-(((2-((4-(4-(((2-(2,4-dioxotetrahydropyrimidin-1 (2H)-yl)-1,3-dioxoisoindolin-5-yl)methyl)(methyl)amino) piperidin-1-yl)phenyl)amino)-5-(trifluoromethyl)pyrimidin-4-yl)amino)methyl)pyrazin-2-yl)-N-methylmethanesulfonamide;
N-(3-(((2-((4-(4-(4-((2-(2,4-dioxotetrahydropyrimidin-1 (2H)-yl)-1,3-dioxoisoindolin-5-yl)methyl)piperazin-1-yl)piperidin-1-yl)phenyl)amino)-5-(trifluoromethyl)pyrimidin-4-yl)amino)methyl)pyrazin-2-yl)-N-methylmethanesulfonamide;
N-(3-(((2-((4-(4-((2-(2,4-dioxotetrahydropyrimidin-1 (2H)-yl)-1,3-dioxoisoindolin-5-yl)methyl)piperazin-1-yl)phenyl)amino)-5-(trifluoromethyl)pyrimidin-4-yl)amino)methyl)phenyl)-N-methylmethanesulfonamide;
N-(3-(((2-((4-(1-((2-(2,4-dioxotetrahydropyrimidin-1 (2H)-yl)-1,3-dioxoisoindolin-5-yl)methyl)piperidin-4-yl)phenyl)amino)-5-(trifluoromethyl)pyrimidin-4-yl)amino)methyl)phenyl)-N-methylmethanesulfonamide;
N-(3-(((2-((4-(4-(((2-(2,4-dioxotetrahydropyrimidin-1 (2H)-yl)-1,3-dioxoisoindolin-5-yl)methyl)(methyl)amino) piperidin-1-yl)phenyl)amino)-5-(trifluoromethyl)pyrimidin-4-yl)amino)methyl)phenyl)-N-methylmethanesulfonamide;
N-(3-(((2-((4-(4-(4-((2-(2,4-dioxotetrahydropyrimidin-1 (2H)-yl)-1,3-dioxoisoindolin-5-yl)methyl)piperazin-1-yl)piperidin-1-yl)phenyl)amino)-5-(trifluoromethyl)pyrimidin-4-yl)amino)methyl)phenyl)-N-methylmethanesulfonamide;
2-(2,4-dioxotetrahydropyrimidin-1 (2H)-yl)-5-((4-(4-((4-((3-(methylsulfonyl)benzyl)amino)-5-(trifluoromethyl)pyrimidin-2-yl)amino)phenyl) piperazin-1-yl)methyl)isoindoline-1,3-dione;
2-(2-((cyclopropylmethyl)amino)pyridin-4-yl)-N-(3-(difluoromethyl)-1-(1-((2-(2,4-dioxotetrahydropyrimidin-1 (2H)-yl)-1,3-dioxoisoindolin-5-yl)methyl)piperidin-4-yl)-1H-pyrazol-4-yl) oxazole-4-carboxamide;
2-(2-((cyclopropylmethyl)amino)pyridin-4-yl)-N-(3-(difluoromethyl)-1-(1-((1-(2-(2,4-dioxotetrahydropyrimidin-1 (2H)-yl)-1,3-dioxoisoindolin-5-yl)piperidin-4-yl)methyl)piperidin-4-yl)-1H-pyrazol-4-yl) oxazole-4-carboxamide;
2-(2-((cyclopropylmethyl)amino)pyridin-4-yl)-N-(3-(difluoromethyl)-1-(1-((1-(2-(2,4-dioxotetrahydropyrimidin-1 (2H)-yl)-1,3-dioxoisoindolin-4-yl)piperidin-4-yl)methyl)piperidin-4-yl)-1H-pyrazol-4-yl) oxazole-4-carboxamide;
2-(2-((cyclopropylmethyl)amino)pyridin-4-yl)-N-(3-(difluoromethyl)-1-(1-((1′-(2-(2,4-dioxotetrahydropyrimidin-1 (2H)-yl)-1,3-dioxoisoindolin-5-yl)-[1,4′-bipiperidin]-4-yl)methyl)piperidin-4-yl)-1H-pyrazol-4-yl) oxazole-4-carboxamide;
2-(2-((cyclopropylmethyl)amino)pyridin-4-yl)-N-(3-(difluoromethyl)-1-(4-(4-((2-(2,4-dioxotetrahydropyrimidin-1 (2H)-yl)-1,3-dioxoisoindolin-5-yl)methyl)piperazin-1-yl)phenyl)-1H-pyrazol-4-yl) oxazole-4-carboxamide;
N-(2-(1-((2-(2,4-dioxotetrahydropyrimidin-1 (2H)-yl)-1,3-dioxoisoindolin-5-yl)methyl)piperidin-4-yl)-6-methoxy-2H-indazol-5-yl)-3-(trifluoromethyl)benzamide;
N-(2-(1-((1-(2-(2,4-dioxotetrahydropyrimidin-1 (2H)-yl)-1,3-dioxoisoindolin-5-yl)piperidin-4-yl)methyl)piperidin-4-yl)-6-methoxy-2H-indazol-5-yl)-3-(trifluoromethyl)benzamide;
N-(2-(1-((2-(2,4-dioxotetrahydropyrimidin-1 (2H)-yl)-1,3-dioxoisoindolin-5-yl)methyl)piperidin-4-yl)-6-methoxy-2H-indazol-5-yl)-6-(trifluoromethyl)picolinamide;
N-(2-(1-((1-(2-(2,4-dioxotetrahydropyrimidin-1 (2H)-yl)-1,3-dioxoisoindolin-5-yl)piperidin-4-yl)methyl)piperidin-4-yl)-6-methoxy-2H-indazol-5-yl)-6-(trifluoromethyl)picolinamide;
N-(2-(1-((1-(2-(2,4-dioxotetrahydropyrimidin-1 (2H)-yl)-1,3-dioxoisoindolin-5-yl)piperidin-4-yl)methyl)piperidin-4-yl)-6-methoxy-2H-indazol-7-yl)-3-(trifluoromethyl)benzamide;
N-(3-(difluoromethyl)-1-(1-((2-(2,4-dioxotetrahydropyrimidin-1 (2H)-yl)-1,3-dioxoisoindolin-5-yl)methyl)piperidin-4-yl)-1H-pyrazol-4-yl)-5-morpholinopyrazolo[1,5-a]pyrimidine-3-carboxamide;
N-(3-(difluoromethyl)-1-(1-((1-(2-(2,4-dioxotetrahydropyrimidin-1 (2H)-yl)-1,3-dioxoisoindolin-4-yl) piperidin-4-yl)methyl)piperidin-4-yl)-1H-pyrazol-4-yl)-5-morpholinopyrazolo[1,5-a]pyrimidine-3-carboxamide;
N-(3-(difluoromethyl)-1-(1-((1-(2-(2,4-dioxotetrahydropyrimidin-1 (2H)-yl)-1,3-dioxoisoindolin-5-yl) piperidin-4-yl)methyl)piperidin-4-yl)-1H-pyrazol-4-yl)-5-morpholinopyrazolo[1,5-a]pyrimidine-3-carboxamide;
5-((1R,4R)-2-oxa-5-azabicyclo[2.2.1]heptan-5-yl)-N-(3-(difluoromethyl)-1-(1-((2-(2,4-dioxotetrahydropyrimidin-1 (2H)-yl)-1,3-dioxoisoindolin-5-yl)methyl)piperidin-4-yl)-1H-pyrazol-4-yl)pyrazolo[1,5-a]pyrimidine-3-carboxamide;
5-((1R,4R)-2-oxa-5-azabicyclo[2.2.1]heptan-5-yl)-N-(3-(difluoromethyl)-1-(1-((1-(2-(2,4-dioxotetrahydropyrimidin-1 (2H)-yl)-1,3-dioxoisoindolin-5-yl)piperidin-4-yl)methyl)piperidin-4-yl)-1H-pyrazol-4-yl)pyrazolo[1,5-a]pyrimidine-3-carboxamide;
5-((1R,4R)-2-oxa-5-azabicyclo[2.2.1]heptan-5-yl)-N-(3-(difluoromethyl)-1-(1-((1-(2-(2,4-dioxotetrahydropyrimidin-1 (2H)-yl)-1,3-dioxoisoindolin-4-yl)piperidin-4-yl)methyl)piperidin-4-yl)-1H-pyrazol-4-yl)pyrazolo[1,5-a]pyrimidine-3-carboxamide;
5-((1R,4R)-2-oxa-5-azabicyclo[2.2.1]heptan-5-yl)-N-(3-(difluoromethyl)-1-((1r,4R)-4-((4-(2-(2,4-dioxotetrahydropyrimidin-1 (2H)-yl)-1,3-dioxoisoindolin-5-yl)piperazin-1-yl)methyl)cyclohexyl)-1H-pyrazol-4-yl)pyrazolo[1,5-a]pyrimidine-3-carboxamide;
5-((1R,4R)-2-oxa-5-azabicyclo[2.2.1]heptan-5-yl)-N-(3-(difluoromethyl)-1-((1S,4R)-4-(((1S,4S)-5-(2-(2,4-dioxotetrahydropyrimidin-1 (2H)-yl)-1,3-dioxoisoindolin-5-yl)-2,5-diazabicyclo[2.2.1]heptan-2-yl)methyl)cyclohexyl)-1H-pyrazol-4-yl)pyrazolo[1,5-a]pyrimidine-3-carboxamide;
5-((1R,4R)-2-oxa-5-azabicyclo[2.2.1]heptan-5-yl)-N-(3-(difluoromethyl)-1-((1R,4R)-4-(((1R,4R)-5-(2-(2,4-dioxotetrahydropyrimidin-1 (2H)-yl)-1,3-dioxoisoindolin-5-yl)-2,5-diazabicyclo[2.2.1]heptan-2-yl)methyl)cyclohexyl)-1H-pyrazol-4-yl)pyrazolo[1,5-a]pyrimidine-3-carboxamide;
5-((1R,4R)-2-oxa-5-azabicyclo[2.2.1]heptan-5-yl)-N-(3-(difluoromethyl)-1-((1r,4R)-4-((4-(1-(2-(2,4-dioxotetrahydropyrimidin-1 (2H)-yl)-1,3-dioxoisoindolin-5-yl)piperidin-4-yl)piperazin-1-yl)methyl)cyclohexyl)-1H-pyrazol-4-yl)pyrazolo[1,5-a]pyrimidine-3-carboxamide;
5-((1R,4R)-2-oxa-5-azabicyclo[2.2.1]heptan-5-yl)-N-(3-(difluoromethyl)-1-((1r,4R)-4-((4-(2-(2,4-dioxotetrahydropyrimidin-1 (2H)-yl)-6-fluoro-1,3-dioxoisoindolin-5-yl)piperazin-1-yl)methyl)cyclohexyl)-1H-pyrazol-4-yl)pyrazolo[1,5-a]pyrimidine-3-carboxamide;
5-((1R,4R)-2-oxa-5-azabicyclo[2.2.1]heptan-5-yl)-N-(3-(difluoromethyl)-1-((1r,4R)-4-((8-(2-(2,4-dioxotetrahydropyrimidin-1 (2H)-yl)-1,3-dioxoisoindolin-5-yl)-3,8-diazabicyclo[3.2.1]octan-3-yl)methyl)cyclohexyl)-1H-pyrazol-4-yl)pyrazolo[1,5-a]pyrimidine-3-carboxamide;
5-((1R,4R)-2-oxa-5-azabicyclo[2.2.1]heptan-5-yl)-N-(3-(difluoromethyl)-1-((1r,4R)-4-((4-(2-(2,4-dioxotetrahydropyrimidin-1 (2H)-yl)-1,3-dioxoisoindolin-4-yl)piperazin-1-yl)methyl)cyclohexyl)-1H-pyrazol-4-yl)pyrazolo[1,5-a]pyrimidine-3-carboxamide;
5-((1R,4R)-2-oxa-5-azabicyclo[2.2.1]heptan-5-yl)-N-(3-(difluoromethyl)-1-((1r,4R)-4-((6-(2-(2,4-dioxotetrahydropyrimidin-1 (2H)-yl)-1,3-dioxoisoindolin-5-yl)-3,6-diazabicyclo[3.1.1]heptan-3-yl)methyl)cyclohexyl)-1H-pyrazol-4-yl)pyrazolo[1,5-a]pyrimidine-3-carboxamide;
5-((1R,4R)-2-oxa-5-azabicyclo[2.2.1]heptan-5-yl)-N-(3-(difluoromethyl)-1-((1r,4R)-4-((4-(2-(2,4-dioxotetrahydropyrimidin-1 (2H)-yl)-7-fluoro-1,3-dioxoisoindolin-5-yl)piperazin-1-yl)methyl)cyclohexyl)-1H-pyrazol-4-yl)pyrazolo[1,5-a]pyrimidine-3-carboxamide;
5-((1R,4R)-2-oxa-5-azabicyclo[2.2.1]heptan-5-yl)-N-(3-(difluoromethyl)-1-((1r,4R)-4-((4-(1-(2-(2,4-dioxotetrahydropyrimidin-1 (2H)-yl)-1,3-dioxoisoindolin-5-yl)-3,3-difluoropiperidin-4-yl)piperazin-1-yl)methyl)cyclohexyl)-1H-pyrazol-4-yl)pyrazolo[1,5-a]pyrimidine-3-carboxamide;
N-(5-((4-((2-(2,4-dioxotetrahydropyrimidin-1 (2H)-yl)-1,3-dioxoisoindolin-5-yl)methyl)piperazin-1-yl)methyl)-1-((1s,4s)-4-(hydroxymethyl)cyclohexyl)-1H-benzo[d]imidazol-2-yl)-3-(trifluoromethyl)benzamide;
N-(5-(4-((2-(2,4-dioxotetrahydropyrimidin-1 (2H)-yl)-1,3-dioxoisoindolin-5-yl)methyl)piperazin-1-yl)-1-((1s,4s)-4-(hydroxymethyl)cyclohexyl)-1H-benzo[d]imidazol-2-yl)-3-(trifluoromethyl)benzamide;
N-(5-(4-((1-(2-(2,4-dioxotetrahydropyrimidin-1 (2H)-yl)-1,3-dioxoisoindolin-5-yl)piperidin-4-yl)methyl)piperazin-1-yl)-1-((1s,4s)-4-(hydroxymethyl)cyclohexyl)-1H-benzo[d]imidazol-2-yl)-3-(trifluoromethyl)benzamide;
N-(5-(4-((1-(2-(2,4-dioxotetrahydropyrimidin-1 (2H)-yl)-1,3-dioxoisoindolin-4-yl)piperidin-4-yl)methyl)piperazin-1-yl)-1-((1s,4s)-4-(hydroxymethyl)cyclohexyl)-1H-benzo[d]imidazol-2-yl)-3-(trifluoromethyl)benzamide;
N-(5-(4-(1-((2-(2,4-dioxotetrahydropyrimidin-1 (2H)-yl)-1,3-dioxoisoindolin-5-yl)methyl)piperidin-4-yl) piperazin-1-yl)-1-((1s,4s)-4-(hydroxymethyl)cyclohexyl)-1H-benzo[d]imidazol-2-yl)-3-(trifluoromethyl)benzamide;
6-(5-cyano-1H-pyrrolo[2,3-b]pyridin-1-yl)-N-(1-((2-(2,4-dioxotetrahydropyrimidin-1 (2H)-yl)-1,3-dioxoisoindolin-5-yl)methyl)piperidin-4-yl)-4-(isopropylamino) nicotinamide;
N-(3-(difluoromethyl)-1-(1-((2-(2,4-dioxotetrahydropyrimidin-1 (2H)-yl)-1,3-dioxoisoindolin-5-yl)methyl)piperidin-4-yl)-1H-pyrazol-4-yl)-2-(2-((2,2,2-trifluoroethyl)amino)pyridin-4-yl) oxazole-4-carboxamide;
N-(3-(difluoromethyl)-1-(1-((1-(2-(2,4-dioxotetrahydropyrimidin-1 (2H)-yl)-1,3-dioxoisoindolin-5-yl) piperidin-4-yl)methyl)piperidin-4-yl)-1H-pyrazol-4-yl)-2-(2-((2,2,2-trifluoroethyl)amino)pyridin-4-yl) oxazole-4-carboxamide;
N-(3-(difluoromethyl)-1-(1-((1-(2-(2,4-dioxotetrahydropyrimidin-1 (2H)-yl)-1,3-dioxoisoindolin-5-yl) piperidin-4-yl)methyl)piperidin-4-yl)-1H-pyrazol-4-yl)-2-(2-((2,2,2-trifluoroethyl)amino)pyridin-4-yl) oxazole-4-carboxamide;
N-(3-(difluoromethyl)-1-(1-((1′-(2-(2,4-dioxotetrahydropyrimidin-1 (2H)-yl)-1,3-dioxoisoindolin-5-yl)-[1,4′-bipiperidin]-4-yl)methyl)piperidin-4-yl)-1H-pyrazol-4-yl)-2-(2-((2,2,2-trifluoroethyl)amino)pyridin-4-yl) oxazole-4-carboxamide;
(R)-N-(2-(4-((2-(2,4-dioxotetrahydropyrimidin-1 (2H)-yl)-1,3-dioxoisoindolin-5-yl)methyl)piperazin-1-yl)-5-(3-hydroxypyrrolidin-1-yl) oxazolo[4,5-b]pyridin-6-yl)-2-(2-methylpyridin-4-yl) oxazole-4-carboxamide;
N-(2-(1-((2-(2,4-dioxotetrahydropyrimidin-1 (2H)-yl)-1,3-dioxoisoindolin-5-yl)methyl)piperidin-4-yl)-5-(2-hydroxypropan-2-yl)benzo[d]thiazol-6-yl)-6-(trifluoromethyl)picolinamide;
N-(2-(1-((2-(2,4-dioxotetrahydropyrimidin-1 (2H)-yl)-1,3-dioxoisoindolin-5-yl)methyl)piperidin-4-yl)-5-(2-hydroxypropan-2-yl)benzo[d]oxazol-6-yl)-6-(trifluoromethyl)picolinamide;
N-(2-(1-((1-(2-(2,4-dioxotetrahydropyrimidin-1 (2H)-yl)-1,3-dioxoisoindolin-5-yl)piperidin-4-yl)methyl)piperidin-4-yl)-5-(2-hydroxypropan-2-yl)benzo[d]oxazol-6-yl)-6-(trifluoromethyl)picolinamide;
N-(2-(1-((1-(2-(2,4-dioxotetrahydropyrimidin-1 (2H)-yl)-1,3-dioxoisoindolin-4-yl)piperidin-4-yl)methyl)piperidin-4-yl)-5-(2-hydroxypropan-2-yl)benzo[d]oxazol-6-yl)-6-(trifluoromethyl)picolinamide;
N-(2-(1-((2-(2,4-dioxotetrahydropyrimidin-1 (2H)-yl)-1,3-dioxoisoindolin-5-yl)methyl)piperidin-4-yl)-6-(2-hydroxypropan-2-yl)-2H-indazol-5-yl)-6-(trifluoromethyl)picolinamide;
N-(2-(1-((1-(2-(2,4-dioxotetrahydropyrimidin-1 (2H)-yl)-1,3-dioxoisoindolin-5-yl)piperidin-4-yl)methyl)piperidin-4-yl)-6-(2-hydroxypropan-2-yl)-2H-indazol-5-yl)-6-(trifluoromethyl)picolinamide;
N-(2-(1-((1-(2-(2,4-dioxotetrahydropyrimidin-1 (2H)-yl)-1,3-dioxoisoindolin-4-yl)piperidin-4-yl)methyl)piperidin-4-yl)-6-(2-hydroxypropan-2-yl)-2H-indazol-5-yl)-6-(trifluoromethyl)picolinamide;
N-(2-(1-((1′-(2-(2,4-dioxotetrahydropyrimidin-1 (2H)-yl)-1,3-dioxoisoindolin-5-yl)-[1,4′-bipiperidin]-4-yl)methyl)piperidin-4-yl)-6-(2-hydroxypropan-2-yl)-2H-indazol-5-yl)-6-(trifluoromethyl)picolinamide;
6-(6-(4-((2-(2,4-dioxotetrahydropyrimidin-1 (2H)-yl)-1,3-dioxoisoindolin-5-yl)methyl)piperazin-1-yl)pyridin-3-yl)-1-isopropyl-N-((6-methyl-2-oxo-4-propyl-1,2-dihydropyridin-3-yl)methyl)-1H-indazole-4-carboxamide;
6-(6-(4-(((2-(2,4-dioxotetrahydropyrimidin-1 (2H)-yl)-1,3-dioxoisoindolin-5-yl)methyl)(methyl)amino) piperidin-1-yl)pyridin-3-yl)-1-isopropyl-N-((6-methyl-2-oxo-4-propyl-1,2-dihydropyridin-3-yl)methyl)-1H-indazole-4-carboxamide;
6-(6-(4-(1-((2-(2,4-dioxotetrahydropyrimidin-1 (2H)-yl)-1,3-dioxoisoindolin-5-yl)methyl)piperidin-4-yl) piperazin-1-yl)pyridin-3-yl)-1-isopropyl-N-((6-methyl-2-oxo-4-propyl-1,2-dihydropyridin-3-yl)methyl)-1H-indazole-4-carboxamide;
N-((4,6-dimethyl-2-oxo-1,2-dihydropyridin-3-yl)methyl)-4′-((4-((2-(2,4-dioxotetrahydropyrimidin-1 (2H)-yl)-1,3-dioxoisoindolin-5-yl)methyl)piperazin-1-yl)methyl)-5-(ethyl(tetrahydro-2H-pyran-4-yl)amino)-4-methyl-[1,1′-biphenyl]-3-carboxamide;
N-((4,6-dimethyl-2-oxo-1,2-dihydropyridin-3-yl)methyl)-4′-(4-((2-(2,4-dioxotetrahydropyrimidin-1 (2H)-yl)-1,3-dioxoisoindolin-5-yl)methyl)piperazin-1-yl)-5-(ethyl(tetrahydro-2H-pyran-4-yl)amino)-4-methyl-[1,1′-biphenyl]-3-carboxamide;
N-((4,6-dimethyl-2-oxo-1,2-dihydropyridin-3-yl)methyl)-4′-(4-(((2-(2,4-dioxotetrahydropyrimidin-1 (2H)-yl)-1,3-dioxoisoindolin-5-yl)methyl)(methyl)amino) piperidin-1-yl)-5-(ethyl(tetrahydro-2H-pyran-4-yl)amino)-4-methyl-[1,1′-biphenyl]-3-carboxamide;
N-((4,6-dimethyl-2-oxo-1,2-dihydropyridin-3-yl)methyl)-4′-(4-(1-((2-(2,4-dioxotetrahydropyrimidin-1 (2H)-yl)-1,3-dioxoisoindolin-5-yl)methyl)piperidin-4-yl)piperazin-1-yl)-5-(ethyl(tetrahydro-2H-pyran-4-yl)amino)-4-methyl-[1,1′-biphenyl]-3-carboxamide;
(R)-4-(4-((4′-chloro-4,4-dimethyl-3,4,5,6-tetrahydro-[1,1′-biphenyl]-2-yl)methyl)piperazin-1-yl)-N-((4-((4-(4-((2-(2,4-dioxotetrahydropyrimidin-1 (2H)-yl)-1,3-dioxoisoindolin-5-yl)methyl)piperazin-1-yl-2,2,3,3,5,5,6,6-d8)-1-(phenylthio)butan-2-yl)amino)-3-((trifluoromethyl)sulfonyl)phenyl)sulfonyl)benzamide;
4-(4-((4′-chloro-4,4-dimethyl-3,4,5,6-tetrahydro-[1,1′-biphenyl]-2-yl)methyl)piperazin-1-yl)-N-((4-(((2R)-4-(4-((2-(2,4-dioxotetrahydropyrimidin-1 (2H)-yl)-1,3-dioxoisoindolin-5-yl)methyl)-3,5-dimethylpiperazin-1-yl)-1-(phenylthio)butan-2-yl)amino)-3-((trifluoromethyl)sulfonyl)phenyl)sulfonyl)benzamide;
4-(4-((4′-chloro-4,4-dimethyl-3,4,5,6-tetrahydro-[1,1′-biphenyl]-2-yl)methyl)piperazin-1-yl)-N-((4-(((2R)-4-(4-((2-(2,4-dioxotetrahydropyrimidin-1 (2H)-yl)-1,3-dioxoisoindolin-5-yl)methyl)-2,6-dimethylpiperazin-1-yl)-1-(phenylthio)butan-2-yl)amino)-3-((trifluoromethyl)sulfonyl)phenyl)sulfonyl)benzamide;
4-(4-((4′-chloro-4,4-dimethyl-3,4,5,6-tetrahydro-[1,1′-biphenyl]-2-yl)methyl)piperazin-1-yl)-N-((4-(((2R)-4-(6-((2-(2,4-dioxotetrahydropyrimidin-1 (2H)-yl)-1,3-dioxoisoindolin-5-yl)methyl)-3,6-diazabicyclo[3.1.1]heptan-3-yl)-1-(phenylthio)butan-2-yl)amino)-3-((trifluoromethyl)sulfonyl)phenyl)sulfonyl)benzamide;
4-(4-((4′-chloro-4,4-dimethyl-3,4,5,6-tetrahydro-[1,1′-biphenyl]-2-yl)methyl)piperazin-1-yl)-N-((4-(((2R)-4-(3-((2-(2,4-dioxotetrahydropyrimidin-1 (2H)-yl)-1,3-dioxoisoindolin-5-yl)methyl)-3,6-diazabicyclo[3.1.1]heptan-6-yl)-1-(phenylthio)butan-2-yl)amino)-3-((trifluoromethyl)sulfonyl)phenyl)sulfonyl)benzamide;
4-(4-((4′-chloro-4,4-dimethyl-3,4,5,6-tetrahydro-[1,1′-biphenyl]-2-yl)methyl)piperazin-1-yl)-N-((4-(((2R)-4-(8-((2-(2,4-dioxotetrahydropyrimidin-1 (2H)-yl)-1,3-dioxoisoindolin-5-yl)methyl)-3,8-diazabicyclo[3.2.1]octan-3-yl)-1-(phenylthio)butan-2-yl)amino)-3-((trifluoromethyl)sulfonyl)phenyl)sulfonyl)benzamide;
4-(4-((4′-chloro-4,4-dimethyl-3,4,5,6-tetrahydro-[1,1′-biphenyl]-2-yl)methyl)piperazin-1-yl)-N-((4-(((2R)-4-(3-((2-(2,4-dioxotetrahydropyrimidin-1 (2H)-yl)-1,3-dioxoisoindolin-5-yl)methyl)-3,8-diazabicyclo[3.2.1]octan-8-yl)-1-(phenylthio)butan-2-yl)amino)-3-((trifluoromethyl)sulfonyl)phenyl)sulfonyl)benzamide;
4-(4-((4′-chloro-4,4-dimethyl-3,4,5,6-tetrahydro-[1,1′-biphenyl]-2-yl)methyl)piperazin-1-yl)-N-((4-(((R)-4-((1R,4R)-5-((2-(2,4-dioxotetrahydropyrimidin-1 (2H)-yl)-1,3-dioxoisoindolin-5-yl)methyl)-2,5-diazabicyclo[2.2.1]heptan-2-yl)-1-(phenylthio)butan-2-yl)amino)-3-((trifluoromethyl)sulfonyl)phenyl)sulfonyl)benzamide;
4-(4-((4′-chloro-4,4-dimethyl-3,4,5,6-tetrahydro-[1,1′-biphenyl]-2-yl)methyl)piperazin-1-yl)-N-((4-(((R)-4-((1S,4S)-5-((2-(2,4-dioxotetrahydropyrimidin-1 (2H)-yl)-1,3-dioxoisoindolin-5-yl)methyl)-2,5-diazabicyclo[2.2.1]heptan-2-yl)-1-(phenylthio)butan-2-yl)amino)-3-((trifluoromethyl)sulfonyl)phenyl)sulfonyl)benzamide;
4-(4-((4′-chloro-4,4-dimethyl-3,4,5,6-tetrahydro-[1,1′-biphenyl]-2-yl)methyl)piperazin-1-yl)-N-((4-(((2R)-4-(5-((2-(2,4-dioxotetrahydropyrimidin-1 (2H)-yl)-1,3-dioxoisoindolin-5-yl)methyl)-2,5-diazabicyclo[2.2.2]octan-2-yl)-1-(phenylthio)butan-2-yl)amino)-3-((trifluoromethyl)sulfonyl)phenyl)sulfonyl)benzamide;
(R)-4-(4-((4′-chloro-4,4-dimethyl-3,4,5,6-tetrahydro-[1,1′-biphenyl]-2-yl)methyl)piperazin-1-yl)-N-((4-((4-(4-((2-(2,4-dioxotetrahydropyrimidin-1 (2H)-yl)-1,3-dioxoisoindolin-5-yl)methyl)piperazin-1-yl)-1-(phenylthio)butan-2-yl)amino)-3-((trifluoromethyl)sulfonyl)phenyl)sulfonyl)benzamide;
2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)-N-((4-(((1-((2-(2,4-dioxotetrahydropyrimidin-1 (2H)-yl)-1,3-dioxoisoindolin-5-yl)methyl)piperidin-4-yl)methyl)amino)-3-nitrophenyl)sulfonyl)benzamide;
(R)-N-(1-(4-(((2-(2,4-dioxotetrahydropyrimidin-1 (2H)-yl)-1,3-dioxoisoindolin-5-yl)methyl)amino)benzyl)-1H-pyrazol-4-yl)-9-methyl-6-oxo-6,7,8,9-tetrahydropyrido[3′,2′: 4,5]pyrrolo[1,2-a]pyrazine-2-carboxamide;
(R)-N-(1-(4-(4-((2-(2,4-dioxotetrahydropyrimidin-1 (2H)-yl)-1,3-dioxoisoindolin-5-yl)methyl)piperazin-1-yl)benzyl)-1H-pyrazol-4-yl)-9-methyl-6-oxo-6,7,8,9-tetrahydropyrido[3′,2′: 4,5]pyrrolo[1,2-a]pyrazine-2-carboxamide;
(R)-N-(1-(4-(4-(((2-(2,4-dioxotetrahydropyrimidin-1 (2H)-yl)-1,3-dioxoisoindolin-5-yl)methyl)(methyl)amino) piperidin-1-yl)benzyl)-1H-pyrazol-4-yl)-9-methyl-6-oxo-6,7,8,9-tetrahydropyrido[3′,2′: 4,5]pyrrolo[1,2-a]pyrazine-2-carboxamide;
(R)-N-(1-(4-(4-(4-((2-(2,4-dioxotetrahydropyrimidin-1 (2H)-yl)-1,3-dioxoisoindolin-5-yl)methyl)piperazin-1-yl) piperidin-1-yl)benzyl)-1H-pyrazol-4-yl)-9-methyl-6-oxo-6,7,8,9-tetrahydropyrido[3′,2′: 4,5]pyrrolo[1,2-alpyrazine-2-carboxamide;
5-((4-(3-amino-6-(2-hydroxyphenyl)pyridazin-4-yl)piperazin-1-yl)methyl)-2-(2,4-dioxotetrahydropyrimidin-1 (2H)-yl)isoindoline-1,3-dione;
5-(4-((4-(3-amino-6-(2-hydroxyphenyl)pyridazin-4-yl)piperazin-1-yl)methyl)piperidin-1-yl)-2-(2,4-dioxotetrahydropyrimidin-1 (2H)-yl)isoindoline-1,3-dione;
4-(4-((4-(3-amino-6-(2-hydroxyphenyl)pyridazin-4-yl)piperazin-1-yl)methyl)piperidin-1-yl)-2-(2,4-dioxotetrahydropyrimidin-1 (2H)-yl)isoindoline-1,3-dione;
5-(4-((4-(3-amino-6-(2-hydroxyphenyl)pyridazin-4-yl)piperazin-1-yl)methyl)-[1,4′-bipiperidin]-1′-yl)-2-(2,4-dioxotetrahydropyrimidin-1 (2H)-yl)isoindoline-1,3-dione;
5-((4-(3-amino-6-(5-fluoro-2-hydroxyphenyl)pyridazin-4-yl)piperazin-1-yl)methyl)-2-(2,4-dioxotetrahydropyrimidin-1 (2H)-yl)isoindoline-1,3-dione;
5-(4-((4-(3-amino-6-(5-fluoro-2-hydroxyphenyl)pyridazin-4-yl)piperazin-1-yl)methyl)piperidin-1-yl)-2-(2,4-dioxotetrahydropyrimidin-1 (2H)-yl)isoindoline-1,3-dione;
4-(4-((4-(3-amino-6-(5-fluoro-2-hydroxyphenyl)pyridazin-4-yl)piperazin-1-yl)methyl)piperidin-1-yl)-2-(2,4-dioxotetrahydropyrimidin-1 (2H)-yl)isoindoline-1,3-dione;
5-(4-((4-(3-amino-6-(5-fluoro-2-hydroxyphenyl)pyridazin-4-yl)piperazin-1-yl)methyl)-[1,4′-bipiperidin]-1′-yl)-2-(2,4-dioxotetrahydropyrimidin-1 (2H)-yl)isoindoline-1,3-dione;
5-(4-(((1-(4-((9-cyclopentyl-8-(phenylamino)-9H-purin-2-yl)amino)phenyl) piperidin-4-yl)(methyl)amino)methyl)piperidin-1-yl)-2-(2,4-dioxotetrahydropyrimidin-1 (2H)-yl)isoindoline-1,3-dione;
4-(4-(((1-(4-((9-cyclopentyl-8-(phenylamino)-9H-purin-2-yl)amino)phenyl) piperidin-4-yl)(methyl)amino)methyl)piperidin-1-yl)-2-(2,4-dioxotetrahydropyrimidin-1 (2H)-yl)isoindoline-1,3-dione;
5-(4-((4-(1-(4-((9-cyclopentyl-8-(phenylamino)-9H-purin-2-yl)amino)phenyl) piperidin-4-yl)piperazin-1-yl)methyl)piperidin-1-yl)-2-(2,4-dioxotetrahydropyrimidin-1 (2H)-yl)isoindoline-1,3-dione;
4-(4-((4-(1-(4-((9-cyclopentyl-8-(phenylamino)-9H-purin-2-yl)amino)phenyl) piperidin-4-yl)piperazin-1-yl)methyl)piperidin-1-yl)-2-(2,4-dioxotetrahydropyrimidin-1 (2H)-yl)isoindoline-1,3-dione;
5-(4-(((1-(4-((5-bromo-4-((5-(dimethylphosphoryl) quinoxalin-6-yl)amino)pyrimidin-2-yl)amino)-5-methoxy-2-methylphenyl) piperidin-4-yl)(methyl)amino)methyl)piperidin-1-yl)-2-(2,4-dioxotetrahydropyrimidin-1 (2H)-yl)isoindoline-1,3-dione;
4-(4-(((1-(4-((5-bromo-4-((5-(dimethylphosphoryl) quinoxalin-6-yl)amino)pyrimidin-2-yl)amino)-5-methoxy-2-methylphenyl) piperidin-4-yl)(methyl)amino)methyl)piperidin-1-yl)-2-(2,4-dioxotetrahydropyrimidin-1 (2H)-yl)isoindoline-1,3-dione;
5-(4-((4-(1-(4-((5-bromo-4-((5-(dimethylphosphoryl) quinoxalin-6-yl)amino)pyrimidin-2-yl)amino)-5-methoxy-2-methylphenyl) piperidin-4-yl)piperazin-1-yl)methyl)piperidin-1-yl)-2-(2,4-dioxotetrahydropyrimidin-1 (2H)-yl)isoindoline-1,3-dione;
4-(4-((4-(1-(4-((5-bromo-4-((5-(dimethylphosphoryl) quinoxalin-6-yl)amino)pyrimidin-2-yl)amino)-5-methoxy-2-methylphenyl) piperidin-4-yl)piperazin-1-yl)methyl)piperidin-1-yl)-2-(2,4-dioxotetrahydropyrimidin-1 (2H)-yl)isoindoline-1,3-dione;
5-(4-((4-(6-((2-(1-(cyclopropylsulfonyl)-1H-pyrazol-4-yl)pyrimidin-4-yl)amino)-1-isopropyl-1H-pyrazolo[4,3-c]pyridin-3-yl)piperazin-1-yl)methyl)piperidin-1-yl)-2-(2,4-dioxotetrahydropyrimidin-1 (2H)-yl)isoindoline-1,3-dione;
2-(2,4-dioxotetrahydropyrimidin-1 (2H)-yl)-5-(4-((4-(1-isopropyl-6-((2-(4-methoxypiperidin-1-yl)pyrimidin-4-yl)amino)-1H-pyrazolo[4,3-c]pyridin-3-yl)piperazin-1-yl)methyl)piperidin-1-yl)isoindoline-1,3-dione;
2-(2,4-dioxotetrahydropyrimidin-1 (2H)-yl)-4-(4-((4-(1-isopropyl-6-((2-(4-methoxypiperidin-1-yl)pyrimidin-4-yl)amino)-1H-pyrazolo[4,3-c]pyridin-3-yl)piperazin-1-yl)methyl)piperidin-1-yl)isoindoline-1,3-dione;
2-(2,4-dioxotetrahydropyrimidin-1 (2H)-yl)-5-(4-(((1-(1-isopropyl-6-((2-(4-methoxypiperidin-1-yl)pyrimidin-4-yl)amino)-1H-pyrazolo[4,3-c]pyridin-3-yl)piperidin-4-yl)(methyl)amino)methyl)piperidin-1-yl)isoindoline-1,3-dione;
2-(2,4-dioxotetrahydropyrimidin-1 (2H)-yl)-4-(4-(((1-(1-isopropyl-6-((2-(4-methoxypiperidin-1-yl)pyrimidin-4-yl)amino)-1H-pyrazolo[4,3-c]pyridin-3-yl)piperidin-4-yl)(methyl)amino)methyl)piperidin-1-yl)isoindoline-1,3-dione;
2-(6,7-dihydro-5H-pyrrolo[1,2-c]imidazol-1-yl)-2-(6-(4-(4-((1-(2-(2,4-dioxotetrahydropyrimidin-1 (2H)-yl)-1,3-dioxoisoindolin-5-yl)piperidin-4-yl)methyl)piperazin-1-yl)phenyl)-4-fluoro-1-oxoisoindolin-2-yl)-N-(thiazol-2-yl)acetamide;
2-(6,7-dihydro-5H-pyrrolo[1,2-c]imidazol-1-yl)-2-(6-(4-(4-((1-(2-(2,4-dioxotetrahydropyrimidin-1 (2H)-yl)-1,3-dioxoisoindolin-4-yl)piperidin-4-yl)methyl)piperazin-1-yl)phenyl)-4-fluoro-1-oxoisoindolin-2-yl)-N-(thiazol-2-yl)acetamide;
2-(6,7-dihydro-5H-pyrrolo[1,2-c]imidazol-1-yl)-2-(6-(4-((1R,4R)-5-((1-(2-(2,4-dioxotetrahydropyrimidin-1 (2H)-yl)-1,3-dioxoisoindolin-5-yl)piperidin-4-yl)methyl)-2,5-diazabicyclo[2.2.1]heptan-2-yl)phenyl)-4-fluoro-1-oxoisoindolin-2-yl)-N-(thiazol-2-yl)acetamide;
2-(6,7-dihydro-5H-pyrrolo[1,2-c]imidazol-1-yl)-2-(6-(4-((1R,4R)-5-((1-(2-(2,4-dioxotetrahydropyrimidin-1 (2H)-yl)-1,3-dioxoisoindolin-4-yl)piperidin-4-yl)methyl)-2,5-diazabicyclo[2.2.1]heptan-2-yl)phenyl)-4-fluoro-1-oxoisoindolin-2-yl)-N-(thiazol-2-yl)acetamide;
4-(((4-(5-chloro-2-((1-((1-(2-(2,4-dioxotetrahydropyrimidin-1 (2H)-yl)-1,3-dioxoisoindolin-5-yl)piperidin-4-yl)methyl)piperidin-4-yl)amino)pyridin-4-yl) thiazol-2-yl)amino)methyl)tetrahydro-2H-pyran-4-carbonitrile;
4-(((4-(5-chloro-2-((1-((1-(2-(2,4-dioxotetrahydropyrimidin-1 (2H)-yl)-1,3-dioxoisoindolin-4-yl)piperidin-4-yl)methyl)piperidin-4-yl)amino)pyridin-4-yl) thiazol-2-yl)amino)methyl)tetrahydro-2H-pyran-4-carbonitrile;
5-(4-((4-((5-chloro-4-(5-(cyclopropylmethyl)-1-methyl-1H-pyrazol-4-yl)pyrimidin-2-yl)amino) piperidin-1-yl)methyl)piperidin-1-yl)-2-(2,4-dioxotetrahydropyrimidin-1 (2H)-yl)isoindoline-1,3-dione;
4-(4-((4-((5-chloro-4-(5-(cyclopropylmethyl)-1-methyl-1H-pyrazol-4-yl)pyrimidin-2-yl)amino) piperidin-1-yl)methyl)piperidin-1-yl)-2-(2,4-dioxotetrahydropyrimidin-1 (2H)-yl)isoindoline-1,3-dione;
4-(((5′-chloro-2′-((1-((1-(2-(2,4-dioxotetrahydropyrimidin-1 (2H)-yl)-1,3-dioxoisoindolin-5-yl)piperidin-4-yl)methyl)piperidin-4-yl)amino)-[2,4′-bipyridin]-6-yl)amino)methyl)tetrahydro-2H-pyran-4-carbonitrile;
4-(((5′-chloro-2′-((1-((1-(2-(2,4-dioxotetrahydropyrimidin-1 (2H)-yl)-1,3-dioxoisoindolin-4-yl)piperidin-4-yl)methyl)piperidin-4-yl)amino)-[2,4′-bipyridin]-6-yl)amino)methyl)tetrahydro-2H-pyran-4-carbonitrile;
N-(5-cyclobutyl-1H-pyrazol-3-yl)-2-(4-(4-(((2-(2,4-dioxotetrahydropyrimidin-1 (2H)-yl)-1-oxoisoindolin-5-yl)methyl)(methyl)amino) piperidin-1-yl)phenyl)acetamide;
N-(5-cyclobutyl-1H-pyrazol-3-yl)-2-(4-(4-(((2-(2,4-dioxotetrahydropyrimidin-1 (2H)-yl)-1,3-dioxoisoindolin-5-yl)methyl)(methyl)amino) piperidin-1-yl)phenyl)acetamide;
N-(5-cyclobutyl-1H-pyrazol-3-yl)-2-(4-(4-((2-(2,4-dioxotetrahydropyrimidin-1 (2H)-yl)-1-oxoisoindolin-5-yl)methyl)piperazin-1-yl)phenyl)acetamide;
N-(5-cyclobutyl-1H-pyrazol-3-yl)-2-(4-(4-((2-(2,4-dioxotetrahydropyrimidin-1 (2H)-yl)-1,3-dioxoisoindolin-5-yl)methyl)piperazin-1-yl)phenyl)acetamide;
N-(5-cyclobutyl-1H-pyrazol-3-yl)-2-(4-(4-((1-(2-(2,4-dioxotetrahydropyrimidin-1 (2H)-yl)-1,3-dioxoisoindolin-5-yl)piperidin-4-yl)methyl)piperazin-1-yl)phenyl)acetamide;
N-(5-cyclobutyl-1H-pyrazol-3-yl)-2-(4-(4-((1-(2-(2,4-dioxotetrahydropyrimidin-1 (2H)-yl)-1,3-dioxoisoindolin-4-yl)piperidin-4-yl)methyl)piperazin-1-yl)phenyl)acetamide;
N-(4-((4-((1-(2-(2,4-dioxotetrahydropyrimidin-1 (2H)-yl)-1,3-dioxoisoindolin-5-yl)piperidin-4-yl)methyl)piperazin-1-yl)methyl)-3-(trifluoromethyl)phenyl)-3-(imidazo[1,2-b]pyridazin-3-ylethynyl)-4-methylbenzamide;
N-(4-((4-((1-(2-(2,4-dioxotetrahydropyrimidin-1 (2H)-yl)-1,3-dioxoisoindolin-4-yl)piperidin-4-yl)methyl)piperazin-1-yl)methyl)-3-(trifluoromethyl)phenyl)-3-(imidazo[1,2-b]pyridazin-3-ylethynyl)-4-methylbenzamide;
N-(4-(4-((1-(2-(2,4-dioxotetrahydropyrimidin-1 (2H)-yl)-1,3-dioxoisoindolin-5-yl)piperidin-4-yl)methyl)piperazin-1-yl)-3-(trifluoromethyl)phenyl)-3-(imidazo[1,2-b]pyridazin-3-ylethynyl)-4-methylbenzamide;
N-(4-(4-((1-(2-(2,4-dioxotetrahydropyrimidin-1 (2H)-yl)-1,3-dioxoisoindolin-4-yl)piperidin-4-yl)methyl)piperazin-1-yl)-3-(trifluoromethyl)phenyl)-3-(imidazo[1,2-b]pyridazin-3-ylethynyl)-4-methylbenzamide;
N-(4-(chlorodifluoromethoxy)phenyl)-6-(4-((1-(2-(2,4-dioxotetrahydropyrimidin-1 (2H)-yl)-1,3-dioxoisoindolin-5-yl)piperidin-4-yl)methyl)piperazin-1-yl)-5-(1H-pyrazol-3-yl) nicotinamide;
N-(4-(chlorodifluoromethoxy)phenyl)-6-(4-((1-(2-(2,4-dioxotetrahydropyrimidin-1 (2H)-yl)-1,3-dioxoisoindolin-4-yl)piperidin-4-yl)methyl)piperazin-1-yl)-5-(1H-pyrazol-3-yl) nicotinamide;
N-(4-(chlorodifluoromethoxy)phenyl)-6-(4-(1-((1-(2-(2,4-dioxotetrahydropyrimidin-1 (2H)-yl)-1,3-dioxoisoindolin-5-yl)piperidin-4-yl)methyl)piperidin-4-yl)piperazin-1-yl)-5-(1H-pyrazol-3-yl) nicotinamide;
N-(4-(chlorodifluoromethoxy)phenyl)-6-(4-(1-((1-(2-(2,4-dioxotetrahydropyrimidin-1 (2H)-yl)-1,3-dioxoisoindolin-4-yl)piperidin-4-yl)methyl)piperidin-4-yl)piperazin-1-yl)-5-(1H-pyrazol-3-yl) nicotinamide;
N-(4-(chlorodifluoromethoxy)phenyl)-6-(4-(4-((1-(2-(2,4-dioxotetrahydropyrimidin-1 (2H)-yl)-1,3-dioxoisoindolin-5-yl)piperidin-4-yl)methyl)piperazin-1-yl)piperidin-1-yl)-5-(1H-pyrazol-3-yl) nicotinamide;
N-(4-(chlorodifluoromethoxy)phenyl)-6-(4-(4-((1-(2-(2,4-dioxotetrahydropyrimidin-1 (2H)-yl)-1,3-dioxoisoindolin-4-yl)piperidin-4-yl)methyl)piperazin-1-yl)piperidin-1-yl)-5-(1H-pyrazol-3-yl) nicotinamide;
7-cyclopentyl-2-((5-(4-((1-(2-(2,4-dioxotetrahydropyrimidin-1 (2H)-yl)-1,3-dioxoisoindolin-5-yl)piperidin-4-yl)methyl)piperazin-1-yl)pyridin-2-yl)amino)-N,N-dimethyl-7H-pyrrolo[2,3-d]pyrimidine-6-carboxamide;
7-cyclopentyl-2-((5-(4-((1-(2-(2,4-dioxotetrahydropyrimidin-1 (2H)-yl)-1,3-dioxoisoindolin-4-yl)piperidin-4-yl)methyl)piperazin-1-yl)pyridin-2-yl)amino)-N,N-dimethyl-7H-pyrrolo[2,3-d]pyrimidine-6-carboxamide;
7-cyclopentyl-2-((5-(4-(((1-(2-(2,4-dioxotetrahydropyrimidin-1 (2H)-yl)-1,3-dioxoisoindolin-5-yl)piperidin-4-yl)methyl)(methyl)amino) piperidin-1-yl)pyridin-2-yl)amino)-N,N-dimethyl-7H-pyrrolo[2,3-d]pyrimidine-6-2-(2,4-dioxotetrahydropyrimidin-1 (2H)-yl)-5-(4-((4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)methyl)piperidin-1-yl)isoindoline-1,3-dione;
2-(2,4-dioxotetrahydropyrimidin-1 (2H)-yl)-4-(4-((4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)methyl)piperidin-1-yl)isoindoline-1,3-dione;
2-(2,4-dioxotetrahydropyrimidin-1 (2H)-yl)-5-(4-(((1-(6-((5-fluoro-4-(4-fluoro-1-isopropyl-2-methyl-1H-benzo[d]imidazol-6-yl)pyrimidin-2-yl)amino)pyridin-3-yl)piperidin-4-yl)(methyl)amino)methyl)piperidin-1-yl)isoindoline-1,3-dione;
2-(2,4-dioxotetrahydropyrimidin-1 (2H)-yl)-4-(4-(((1-(6-((5-fluoro-4-(4-fluoro-1-isopropyl-2-methyl-1H-benzo[d]imidazol-6-yl)pyrimidin-2-yl)amino)pyridin-3-yl)piperidin-4-yl)(methyl)amino)methyl)piperidin-1-yl)isoindoline-1,3-dione;
7-cyclopentyl-2-((5-(4-((3-(2-(2,4-dioxotetrahydropyrimidin-1 (2H)-yl)-1,3-dioxoisoindolin-5-yl)-3,6-diazabicyclo[3.1.1]heptan-6-yl)methyl)piperidin-1-yl)pyridin-2-yl)amino)-N,N-dimethyl-7H-pyrrolo[2,3-d]pyrimidine-6-carboxamide;
7-cyclopentyl-2-((5-(4-((3-(2-(2,4-dioxotetrahydropyrimidin-1 (2H)-yl)-1,3-dioxoisoindolin-5-yl)-3,8-diazabicyclo[3.2.1]octan-8-yl)methyl)piperidin-1-yl)pyridin-2-yl)amino)-N,N-dimethyl-7H-pyrrolo[2,3-d]pyrimidine-6-carboxamide;
7-cyclopentyl-2-((5-(4-((5-(2-(2,4-dioxotetrahydropyrimidin-1 (2H)-yl)-1,3-dioxoisoindolin-5-yl)-2,5-diazabicyclo[2.2.2]octan-2-yl)methyl)piperidin-1-yl)pyridin-2-yl)amino)-N,N-dimethyl-7H-pyrrolo[2,3-d]pyrimidine-6-carboxamide;
8-(4-(4-((1-(2-(2,4-dioxotetrahydropyrimidin-1 (2H)-yl)-1,3-dioxoisoindolin-5-yl)piperidin-4-yl)methyl)piperazin-1-yl)piperidin-1-yl)-9-ethyl-6,6-dimethyl-11-oxo-6,11-dihydro-5H-benzo[b]carbazole-3-carbonitrile;
8-(4-(4-((1-(2-(2,4-dioxotetrahydropyrimidin-1 (2H)-yl)-1,3-dioxoisoindolin-4-yl)piperidin-4-yl)methyl)piperazin-1-yl)piperidin-1-yl)-9-ethyl-6,6-dimethyl-11-oxo-6,11-dihydro-5H-benzo[b]carbazole-3-carbonitrile;
8-(4-((1-(2-(2,4-dioxotetrahydropyrimidin-1 (2H)-yl)-1,3-dioxoisoindolin-5-yl)piperidin-4-yl)methyl)piperazin-1-yl)-9-ethyl-6,6-dimethyl-11-oxo-6,11-dihydro-5H-benzo[b]carbazole-3-carbonitrile;
8-(4-((1-(2-(2,4-dioxotetrahydropyrimidin-1 (2H)-yl)-1,3-dioxoisoindolin-4-yl)piperidin-4-yl)methyl)piperazin-1-yl)-9-ethyl-6,6-dimethyl-11-oxo-6,11-dihydro-5H-benzo[b]carbazole-3-carbonitrile;
1-(5-((4-(4-((5-chloro-4-((2-(isopropylsulfonyl)phenyl)amino)pyrimidin-2-yl)amino)-5-isopropoxy-2-methylphenyl) piperidin-1-yl)methyl)-1-oxoisoindolin-2-yl)dihydropyrimidine-2,4 (1H,3H)-dione;
5-((4-(4-((5-chloro-4-((2-(isopropylsulfonyl)phenyl)amino)pyrimidin-2-yl)amino)-5-isopropoxy-2-methylphenyl) piperidin-1-yl)methyl)-2-(2,4-dioxotetrahydropyrimidin-1 (2H)-yl)isoindoline-1,3-dione;
5-(4-((4-(4-((5-chloro-4-((2-(isopropylsulfonyl)phenyl)amino)pyrimidin-2-yl)amino)-5-isopropoxy-2-methylphenyl) piperidin-1-yl)methyl)piperidin-1-yl)-2-(2,4-dioxotetrahydropyrimidin-1 (2H)-yl)isoindoline-1,3-dione;
4-(4-((4-(4-((5-chloro-4-((2-(isopropylsulfonyl)phenyl)amino)pyrimidin-2-yl)amino)-5-isopropoxy-2-methylphenyl) piperidin-1-yl)methyl)piperidin-1-yl)-2-(2,4-dioxotetrahydropyrimidin-1 (2H)-yl)isoindoline-1,3-dione;
5-(4-((4-(1-(4-((5-chloro-4-((2-(dimethylphosphoryl)phenyl)amino)pyrimidin-2-yl)amino)-3-methoxyphenyl) piperidin-4-yl)piperazin-1-yl)methyl)piperidin-1-yl)-2-(2,4-dioxotetrahydropyrimidin-1 (2H)-yl)isoindoline-1,3-dione;
4-(4-((4-(1-(4-((5-chloro-4-((2-(dimethylphosphoryl)phenyl)amino)pyrimidin-2-yl)amino)-3-methoxyphenyl) piperidin-4-yl)piperazin-1-yl)methyl)piperidin-1-yl)-2-(2,4-dioxotetrahydropyrimidin-1 (2H)-yl)isoindoline-1,3-dione;
5-(4-((4-(4-((5-chloro-4-((2-(dimethylphosphoryl)phenyl)amino)pyrimidin-2-yl)amino)-3-methoxyphenyl) piperazin-1-yl)methyl)piperidin-1-yl)-2-(2,4-dioxotetrahydropyrimidin-1 (2H)-yl)isoindoline-1,3-dione;
4-(4-((4-(4-((5-chloro-4-((2-(dimethylphosphoryl)phenyl)amino)pyrimidin-2-yl)amino)-3-methoxyphenyl) piperazin-1-yl)methyl)piperidin-1-yl)-2-(2,4-dioxotetrahydropyrimidin-1 (2H)-yl)isoindoline-1,3-dione;
5-(4-(((1-(4-((5-chloro-4-((2-(dimethylphosphoryl)phenyl)amino)pyrimidin-2-yl)amino)-3-methoxyphenyl) piperidin-4-yl)(methyl)amino)methyl)piperidin-1-yl)-2-(2,4-dioxotetrahydropyrimidin-1 (2H)-yl)isoindoline-1,3-dione;
4-(4-(((1-(4-((5-chloro-4-((2-(dimethylphosphoryl)phenyl)amino)pyrimidin-2-yl)amino)-3-methoxyphenyl) piperidin-4-yl)(methyl)amino)methyl)piperidin-1-yl)-2-(2,4-dioxotetrahydropyrimidin-1 (2H)-yl)isoindoline-1,3-dione;
5-(4-((1-(4-((5-chloro-4-((2-(dimethylphosphoryl)phenyl)amino)pyrimidin-2-yl)amino)-3-methoxyphenyl) piperidin-4-yl)methyl)piperazin-1-yl)-2-(2,4-dioxotetrahydropyrimidin-1 (2H)-yl)isoindoline-1,3-dione;
5-((1S,4S)-5-((1-(4-((5-chloro-4-((2-(dimethylphosphoryl)phenyl)amino)pyrimidin-2-yl)amino)-3-methoxyphenyl) piperidin-4-yl)methyl)-2,5-diazabicyclo[2.2.1]heptan-2-yl)-2-(2,4-dioxotetrahydropyrimidin-1 (2H)-yl)isoindoline-1,3-dione;
5-((1R,4R)-5-((1-(4-((5-chloro-4-((2-(dimethylphosphoryl)phenyl)amino)pyrimidin-2-yl)amino)-3-methoxyphenyl) piperidin-4-yl)methyl)-2,5-diazabicyclo[2.2.1]heptan-2-yl)-2-(2,4-dioxotetrahydropyrimidin-1 (2H)-yl)isoindoline-1,3-dione;
5-(4-((1-(4-((5-chloro-4-((2-(dimethylphosphoryl)phenyl)amino)pyrimidin-2-yl)amino)-3-methoxyphenyl) piperidin-4-yl)methyl)piperazin-1-yl)-2-(2,4-dioxotetrahydropyrimidin-1 (2H)-yl)-6-fluoroisoindoline-1,3-dione;
5-(3-((1-(4-((5-chloro-4-((2-(dimethylphosphoryl)phenyl)amino)pyrimidin-2-yl)amino)-3-methoxyphenyl) piperidin-4-yl)methyl)-3,8-diazabicyclo[3.2.1]octan-8-yl)-2-(2,4-dioxotetrahydropyrimidin-1 (2H)-yl)isoindoline-1,3-dione;
N-(3-(((2-((4-(4-((1-(2-(2,4-dioxotetrahydropyrimidin-1 (2H)-yl)-1,3-dioxoisoindolin-5-yl)piperidin-4-yl)methyl)piperazin-1-yl)phenyl)amino)-5-(trifluoromethyl)pyrimidin-4-yl)amino)methyl)pyrazin-2-yl)-N-methylmethanesulfonamide;
N-(3-(((2-((4-(4-((1-(2-(2,4-dioxotetrahydropyrimidin-1 (2H)-yl)-1,3-dioxoisoindolin-4-yl)piperidin-4-yl)methyl)piperazin-1-yl)phenyl)amino)-5-(trifluoromethyl)pyrimidin-4-yl)amino)methyl)pyrazin-2-yl)-N-methylmethanesulfonamide;
N-(3-(((2-((4-(4-(4-((1-(2-(2,4-dioxotetrahydropyrimidin-1 (2H)-yl)-1,3-dioxoisoindolin-4-yl)piperidin-4-yl)methyl)piperazin-1-yl)piperidin-1-yl)phenyl)amino)-5-(trifluoromethyl)pyrimidin-4-yl)amino)methyl)pyrazin-2-yl)-N-methylmethanesulfonamide;
2-(2,4-dioxotetrahydropyrimidin-1 (2H)-yl)-5-(4-((4-(4-((4-((3-(methylsulfonyl)benzyl)amino)-5-(trifluoromethyl)pyrimidin-2-yl)amino)phenyl) piperazin-1-yl)methyl)piperidin-1-yl)isoindoline-1,3-dione;
2-(2,4-dioxotetrahydropyrimidin-1 (2H)-yl)-4-(4-((4-(4-((4-((3-(methylsulfonyl)benzyl)amino)-5-(trifluoromethyl)pyrimidin-2-yl)amino)phenyl) piperazin-1-yl)methyl)piperidin-1-yl)isoindoline-1,3-dione;
N-(3-(((2-((4-(4-((1-(2-(2,4-dioxotetrahydropyrimidin-1 (2H)-yl)-1,3-dioxoisoindolin-5-yl)piperidin-4-yl)methyl)piperazin-1-yl)phenyl)amino)-5-(trifluoromethyl)pyrimidin-4-yl)amino)methyl)phenyl)-N-methylmethanesulfonamide;
N-(3-(((2-((4-(4-((1-(2-(2,4-dioxotetrahydropyrimidin-1 (2H)-yl)-1,3-dioxoisoindolin-4-yl)piperidin-4-yl)methyl)piperazin-1-yl)phenyl)amino)-5-(trifluoromethyl)pyrimidin-4-yl)amino)methyl)phenyl)-N-methylmethanesulfonamide;
N-(3-(((2-((4-(4-(4-((1-(2-(2,4-dioxotetrahydropyrimidin-1 (2H)-yl)-1,3-dioxoisoindolin-4-yl)piperidin-4-yl)methyl)piperazin-1-yl)piperidin-1-yl)phenyl)amino)-5-(trifluoromethyl)pyrimidin-4-yl)amino)methyl)phenyl)-N-methylmethanesulfonamide;
2-((2-((4-(1-((1-(2-(2,4-dioxotetrahydropyrimidin-1 (2H)-yl)-1,3-dioxoisoindolin-5-yl)piperidin-4-yl)methyl)piperidin-4-yl)-2-isopropoxy-5-methylphenyl)amino)-5-(trifluoromethyl)pyridin-4-yl)amino)-N-methylbenzamide;
2-((2-((4-(1-((1-(2-(2,4-dioxotetrahydropyrimidin-1 (2H)-yl)-1,3-dioxoisoindolin-4-yl)piperidin-4-yl)methyl)piperidin-4-yl)-2-isopropoxy-5-methylphenyl)amino)-5-(trifluoromethyl)pyridin-4-yl)amino)-N-methylbenzamide;
6-(5-cyano-1H-pyrrolo[2,3-b]pyridin-1-yl)-N-(1-((1-(2-(2,4-dioxotetrahydropyrimidin-1 (2H)-yl)-1,3-dioxoisoindolin-5-yl)piperidin-4-yl)methyl)piperidin-4-yl)-4-(isopropylamino) nicotinamide;
6-(5-cyano-1H-pyrrolo[2,3-b]pyridin-1-yl)-N-(1-((1-(2-(2,4-dioxotetrahydropyrimidin-1 (2H)-yl)-1,3-dioxoisoindolin-4-yl)piperidin-4-yl)methyl)piperidin-4-yl)-4-(isopropylamino) nicotinamide;
N-(2-(1-((1-(2-(2,4-dioxotetrahydropyrimidin-1 (2H)-yl)-1,3-dioxoisoindolin-4-yl)piperidin-4-yl)methyl)piperidin-4-yl)-6-methoxy-2H-indazol-5-yl)-3-(trifluoromethyl)benzamide;
N-(2-(1-((1-(2-(2,4-dioxotetrahydropyrimidin-1 (2H)-yl)-1,3-dioxoisoindolin-4-yl)piperidin-4-yl)methyl)piperidin-4-yl)-6-methoxy-2H-indazol-5-yl)-6-(trifluoromethyl)picolinamide;
N-(2-(1-((1-(2-(2,4-dioxotetrahydropyrimidin-1 (2H)-yl)-1,3-dioxoisoindolin-4-yl)piperidin-4-yl)methyl)piperidin-4-yl)-5-(2-hydroxypropan-2-yl)benzo[d]thiazol-6-yl)-6-(trifluoromethyl)picolinamide;
N-(2-(1-((1-(2-(2,4-dioxotetrahydropyrimidin-1 (2H)-yl)-1,3-dioxoisoindolin-5-yl)piperidin-4-yl)methyl)piperidin-4-yl)-5-(2-hydroxypropan-2-yl)benzo[d]thiazol-6-yl)-6-(trifluoromethyl)picolinamide;
N-(2-(1-((1′-(2-(2,4-dioxotetrahydropyrimidin-1 (2H)-yl)-1,3-dioxoisoindolin-5-yl)-[1,4′-bipiperidin]-4-yl)methyl)piperidin-4-yl)-5-(2-hydroxypropan-2-yl)benzo[d]oxazol-6-yl)-6-(trifluoromethyl)picolinamide;
N-(3-(difluoromethyl)-1-(1-((1′-(2-(2,4-dioxotetrahydropyrimidin-1 (2H)-yl)-1,3-dioxoisoindolin-5-yl)-[1,4′-bipiperidin]-4-yl)methyl)piperidin-4-yl)-1H-pyrazol-4-yl)-5-morpholinopyrazolo[1,5-a]pyrimidine-3-carboxamide
5-((1R,4R)-2-oxa-5-azabicyclo[2.2.1]heptan-5-yl)-N-(3-(difluoromethyl)-1-(1-((1′-(2-(2,4-dioxotetrahydropyrimidin-1 (2H)-yl)-1,3-dioxoisoindolin-5-yl)-[1,4′-bipiperidin]-4-yl)methyl)piperidin-4-yl)-1H-pyrazol-4-yl)pyrazolo[1,5-a]pyrimidine-3-carboxamide;
(R)-N-(2-(4-((1-(2-(2,4-dioxotetrahydropyrimidin-1 (2H)-yl)-1,3-dioxoisoindolin-5-yl)piperidin-4-yl)methyl)piperazin-1-yl)-5-(3-hydroxypyrrolidin-1-yl) oxazolo[4,5-b]pyridin-6-yl)-2-(2-methylpyridin-4-yl) oxazole-4-carboxamide;
(R)-N-(2-(4-((1-(2-(2,4-dioxotetrahydropyrimidin-1 (2H)-yl)-1,3-dioxoisoindolin-4-yl)piperidin-4-yl)methyl)piperazin-1-yl)-5-(3-hydroxypyrrolidin-1-yl) oxazolo[4,5-b]pyridin-6-yl)-2-(2-methylpyridin-4-yl) oxazole-4-carboxamide;
N-(1-(1-((1-(2-(2,4-dioxotetrahydropyrimidin-1 (2H)-yl)-1,3-dioxoisoindolin-5-yl)piperidin-4-yl)methyl)piperidin-4-yl)-6-methoxy-1H-indazol-7-yl)-3-(trifluoromethyl)benzamide;
N-(2-((1r,4r)-4-((4-(2-(2,4-dioxotetrahydropyrimidin-1 (2H)-yl)-1,3-dioxoisoindolin-5-yl)piperazin-1-yl)methyl)cyclohexyl)-6-methoxy-2H-indazol-5-yl)-6-(trifluoromethyl)picolinamide;
N-(2-((1S,4r)-4-(((1S,4S)-5-(2-(2,4-dioxotetrahydropyrimidin-1 (2H)-yl)-1,3-dioxoisoindolin-5-yl)-2,5-diazabicyclo[2.2.1]heptan-2-yl)methyl)cyclohexyl)-6-methoxy-2H-indazol-5-yl)-6-(trifluoromethyl)picolinamide;
N-(2-((1R,4r)-4-(((1R,4R)-5-(2-(2,4-dioxotetrahydropyrimidin-1 (2H)-yl)-1,3-dioxoisoindolin-5-yl)-2,5-diazabicyclo[2.2.1]heptan-2-yl)methyl)cyclohexyl)-6-methoxy-2H-indazol-5-yl)-6-(trifluoromethyl)picolinamide;
N-(2-((1r,4r)-4-((4-(2-(2,4-dioxotetrahydropyrimidin-1 (2H)-yl)-6-fluoro-1,3-dioxoisoindolin-5-yl)piperazin-1-yl)methyl)cyclohexyl)-6-methoxy-2H-indazol-5-yl)-6-(trifluoromethyl)picolinamide;
N-(2-((1r,4r)-4-((8-(2-(2,4-dioxotetrahydropyrimidin-1 (2H)-yl)-1,3-dioxoisoindolin-5-yl)-3,8-diazabicyclo[3.2.1]octan-3-yl)methyl)cyclohexyl)-6-methoxy-2H-indazol-5-yl)-6-(trifluoromethyl)picolinamide;
N-(2-((1r,4r)-4-((3-(2-(2,4-dioxotetrahydropyrimidin-1 (2H)-yl)-1,3-dioxoisoindolin-5-yl)-3,6-diazabicyclo[3.1.1]heptan-6-yl)methyl)cyclohexyl)-6-methoxy-2H-indazol-5-yl)-6-(trifluoromethyl)picolinamide;
N-(2-((1r,4r)-4-((4-(2-(2,4-dioxotetrahydropyrimidin-1 (2H)-yl)-1,3-dioxoisoindolin-5-yl)piperazin-1-yl)methyl)cyclohexyl)-6-methoxy-2H-indazol-5-yl)-3-(trifluoromethyl)benzamide;
N-(2-((1S,4r)-4-(((1S,4S)-5-(2-(2,4-dioxotetrahydropyrimidin-1 (2H)-yl)-1,3-dioxoisoindolin-5-yl)-2,5-diazabicyclo[2.2.1]heptan-2-yl)methyl)cyclohexyl)-6-methoxy-2H-indazol-5-yl)-3-(trifluoromethyl)benzamide;
N-(2-((1R,4r)-4-(((1R,4R)-5-(2-(2,4-dioxotetrahydropyrimidin-1 (2H)-yl)-1,3-dioxoisoindolin-5-yl)-2,5-diazabicyclo[2.2.1]heptan-2-yl)methyl)cyclohexyl)-6-methoxy-2H-indazol-5-yl)-3-(trifluoromethyl)benzamide;
N-(2-((1r,4r)-4-((4-(2-(2,4-dioxotetrahydropyrimidin-1 (2H)-yl)-6-fluoro-1,3-dioxoisoindolin-5-yl)piperazin-1-yl)methyl)cyclohexyl)-6-methoxy-2H-indazol-5-yl)-3-(trifluoromethyl)benzamide;
N-(2-((1r,4r)-4-((8-(2-(2,4-dioxotetrahydropyrimidin-1 (2H)-yl)-1,3-dioxoisoindolin-5-yl)-3,8-diazabicyclo[3.2.1]octan-3-yl)methyl)cyclohexyl)-6-methoxy-2H-indazol-5-yl)-3-(trifluoromethyl)benzamide;
N-(2-((1r,4r)-4-((3-(2-(2,4-dioxotetrahydropyrimidin-1 (2H)-yl)-1,3-dioxoisoindolin-5-yl)-3,6-diazabicyclo[3.1.1]heptan-6-yl)methyl)cyclohexyl)-6-methoxy-2H-indazol-5-yl)-3-(trifluoromethyl)benzamide;
N-(2-((1r,4r)-4-((3-(2-(2,4-dioxotetrahydropyrimidin-1 (2H)-yl)-1,3-dioxoisoindolin-5-yl)-3,8-diazabicyclo[3.2.1]octan-8-yl)methyl)cyclohexyl)-6-methoxy-2H-indazol-5-yl)-6-(trifluoromethyl)picolinamide;
N-(2-((1r,4r)-4-((4-(2-(2,4-dioxotetrahydropyrimidin-1 (2H)-yl)-1,3-dioxoisoindolin-4-yl)piperazin-1-yl)methyl)cyclohexyl)-6-methoxy-2H-indazol-5-yl)-6-(trifluoromethyl)picolinamide;
N-(2-((1r,4r)-4-((4-(2-(2,4-dioxotetrahydropyrimidin-1 (2H)-yl)-4-fluoro-1,3-dioxoisoindolin-5-yl)piperazin-1-yl)methyl)cyclohexyl)-6-methoxy-2H-indazol-5-yl)-6-(trifluoromethyl)picolinamide;
N-(2-((1r,4r)-4-((4-(2-(2,4-dioxotetrahydropyrimidin-1 (2H)-yl)-7-fluoro-1,3-dioxoisoindolin-5-yl)piperazin-1-yl)methyl)cyclohexyl)-6-methoxy-2H-indazol-5-yl)-6-(trifluoromethyl)picolinamide;
N-(2-((1r,4r)-4-((4-(1-(2-(2,4-dioxotetrahydropyrimidin-1 (2H)-yl)-1,3-dioxoisoindolin-5-yl)piperidin-4-yl) piperazin-1-yl)methyl)cyclohexyl)-6-methoxy-2H-indazol-5-yl)-6-(trifluoromethyl)picolinamide;
N-(2-((1r,4r)-4-((4-(1-(2-(2,4-dioxotetrahydropyrimidin-1 (2H)-yl)-1,3-dioxoisoindolin-5-yl)-3,3-difluoropiperidin-4-yl)piperazin-1-yl)methyl)cyclohexyl)-6-methoxy-2H-indazol-5-yl)-6-(trifluoromethyl)picolinamide;
N-(2-((1r,4r)-4-((5-(2-(2,4-dioxotetrahydropyrimidin-1 (2H)-yl)-1,3-dioxoisoindolin-5-yl)-2,5-diazabicyclo[2.2.2]octan-2-yl)methyl)cyclohexyl)-6-methoxy-2H-indazol-5-yl)-6-(trifluoromethyl)picolinamide;
N-(2-((1r,4r)-4-((3-(2-(2,4-dioxotetrahydropyrimidin-1 (2H)-yl)-1,3-dioxoisoindolin-5-yl)-3,8-diazabicyclo[3.2.1]octan-8-yl)methyl)cyclohexyl)-6-methoxy-2H-indazol-5-yl)-3-(trifluoromethyl)benzamide;
N-(2-((1r,4r)-4-((4-(2-(2,4-dioxotetrahydropyrimidin-1 (2H)-yl)-1,3-dioxoisoindolin-4-yl)piperazin-1-yl)methyl)cyclohexyl)-6-methoxy-2H-indazol-5-yl)-3-(trifluoromethyl)benzamide;
N-(2-((1r,4r)-4-((4-(2-(2,4-dioxotetrahydropyrimidin-1 (2H)-yl)-4-fluoro-1,3-dioxoisoindolin-5-yl)piperazin-1-yl)methyl)cyclohexyl)-6-methoxy-2H-indazol-5-yl)-3-(trifluoromethyl)benzamide;
N-(2-((1r,4r)-4-((4-(2-(2,4-dioxotetrahydropyrimidin-1 (2H)-yl)-7-fluoro-1,3-dioxoisoindolin-5-yl)piperazin-1-yl)methyl)cyclohexyl)-6-methoxy-2H-indazol-5-yl)-3-(trifluoromethyl)benzamide;
N-(2-((1r,4r)-4-((4-(1-(2-(2,4-dioxotetrahydropyrimidin-1 (2H)-yl)-1,3-dioxoisoindolin-5-yl)piperidin-4-yl) piperazin-1-yl)methyl)cyclohexyl)-6-methoxy-2H-indazol-5-yl)-3-(trifluoromethyl)benzamide;
N-(2-((1r,4r)-4-((4-(1-(2-(2,4-dioxotetrahydropyrimidin-1 (2H)-yl)-1,3-dioxoisoindolin-5-yl)-3,3-difluoropiperidin-4-yl)piperazin-1-yl)methyl)cyclohexyl)-6-methoxy-2H-indazol-5-yl)-3-(trifluoromethyl)benzamide;
N-(2-((1r,4r)-4-((5-(2-(2,4-dioxotetrahydropyrimidin-1 (2H)-yl)-1,3-dioxoisoindolin-5-yl)-2,5-diazabicyclo[2.2.2]octan-2-yl)methyl)cyclohexyl)-6-methoxy-2H-indazol-5-yl)-3-(trifluoromethyl)benzamide;
5-((1R,4R)-2-oxa-5-azabicyclo[2.2.1]heptan-5-yl)-N-(3-(difluoromethyl)-1-((1r,4R)-4-((3-(2-(2,4-dioxotetrahydropyrimidin-1 (2H)-yl)-1,3-dioxoisoindolin-5-yl)-3,6-diazabicyclo[3.1.1]heptan-6-yl)methyl)cyclohexyl)-1H-pyrazol-4-yl)pyrazolo[1,5-a]pyrimidine-3-carboxamide;
5-((1R,4R)-2-oxa-5-azabicyclo[2.2.1]heptan-5-yl)-N-(3-(difluoromethyl)-1-((1r,4R)-4-((3-(2-(2,4-dioxotetrahydropyrimidin-1 (2H)-yl)-1,3-dioxoisoindolin-5-yl)-3,8-diazabicyclo[3.2.1]octan-8-yl)methyl)cyclohexyl)-1H-pyrazol-4-yl)pyrazolo[1,5-a]pyrimidine-3-carboxamide;
5-((1R,4R)-2-oxa-5-azabicyclo[2.2.1]heptan-5-yl)-N-(3-(difluoromethyl)-1-((1r,4R)-4-((4-(2-(2,4-dioxotetrahydropyrimidin-1 (2H)-yl)-4-fluoro-1,3-dioxoisoindolin-5-yl)piperazin-1-yl)methyl)cyclohexyl)-1H-pyrazol-4-yl)pyrazolo[1,5-a]pyrimidine-3-carboxamide;
5-((1R,4R)-2-oxa-5-azabicyclo[2.2.1]heptan-5-yl)-N-(3-(difluoromethyl)-1-((1r,4R)-4-((5-(2-(2,4-dioxotetrahydropyrimidin-1 (2H)-yl)-1,3-dioxoisoindolin-5-yl)-2,5-diazabicyclo[2.2.2]octan-2-yl)methyl)cyclohexyl)-1H-pyrazol-4-yl)pyrazolo[1,5-a]pyrimidine-3-carboxamide;
6-(6-(4-((1-(2-(2,4-dioxotetrahydropyrimidin-1 (2H)-yl)-1,3-dioxoisoindolin-5-yl)piperidin-4-yl)methyl)piperazin-1-yl)pyridin-3-yl)-1-isopropyl-N-((6-methyl-2-oxo-4-propyl-1,2-dihydropyridin-3-yl)methyl)-1H-indazole-4-carboxamide;
6-(6-(4-((1-(2-(2,4-dioxotetrahydropyrimidin-1 (2H)-yl)-1,3-dioxoisoindolin-4-yl)piperidin-4-yl)methyl)piperazin-1-yl)pyridin-3-yl)-1-isopropyl-N-((6-methyl-2-oxo-4-propyl-1,2-dihydropyridin-3-yl)methyl)-1H-indazole-4-carboxamide;
N-((4,6-dimethyl-2-oxo-1,2-dihydropyridin-3-yl)methyl)-4′-((4-((1-(2-(2,4-dioxotetrahydropyrimidin-1 (2H)-yl)-1,3-dioxoisoindolin-5-yl)piperidin-4-yl)methyl)piperazin-1-yl)methyl)-5-(ethyl(tetrahydro-2H-pyran-4-yl)amino)-4-methyl-[1,1′-biphenyl]-3-carboxamide;
N-((4,6-dimethyl-2-oxo-1,2-dihydropyridin-3-yl)methyl)-4′-((4-((1-(2-(2,4-dioxotetrahydropyrimidin-1 (2H)-yl)-1,3-dioxoisoindolin-4-yl)piperidin-4-yl)methyl)piperazin-1-yl)methyl)-5-(ethyl(tetrahydro-2H-pyran-4-yl)amino)-4-methyl-[1,1′-biphenyl]-3-carboxamide;
N-((4,6-dimethyl-2-oxo-1,2-dihydropyridin-3-yl)methyl)-4′-(4-((1-(2-(2,4-dioxotetrahydropyrimidin-1 (2H)-yl)-1,3-dioxoisoindolin-5-yl)piperidin-4-yl)methyl)piperazin-1-yl)-5-(ethyl(tetrahydro-2H-pyran-4-yl)amino)-4-methyl-[1,1′-biphenyl]-3-carboxamide;
N-((4,6-dimethyl-2-oxo-1,2-dihydropyridin-3-yl)methyl)-4′-(4-((1-(2-(2,4-dioxotetrahydropyrimidin-1 (2H)-yl)-1,3-dioxoisoindolin-4-yl)piperidin-4-yl)methyl)piperazin-1-yl)-5-(ethyl(tetrahydro-2H-pyran-4-yl)amino)-4-methyl-[1,1′-biphenyl]-3-carboxamide;
N-((4,6-dimethyl-2-oxo-1,2-dihydropyridin-3-yl)methyl)-4′-(4-(((1-(2-(2,4-dioxotetrahydropyrimidin-1 (2H)-yl)-1,3-dioxoisoindolin-5-yl)piperidin-4-yl)methyl)(methyl)amino) piperidin-1-yl)-5-(ethyl(tetrahydro-2H-pyran-4-yl)amino)-4-methyl-[1,1′-biphenyl]-3-carboxamide;
N-((4,6-dimethyl-2-oxo-1,2-dihydropyridin-3-yl)methyl)-4′-(4-(((1-(2-(2,4-dioxotetrahydropyrimidin-1 (2H)-yl)-1,3-dioxoisoindolin-4-yl)piperidin-4-yl)methyl)(methyl)amino) piperidin-1-yl)-5-(ethyl(tetrahydro-2H-pyran-4-yl)amino)-4-methyl-[1,1′-biphenyl]-3-carboxamide;
N-((4,6-dimethyl-2-oxo-1,2-dihydropyridin-3-yl)methyl)-4′-(4-(1-((1-(2-(2,4-dioxotetrahydropyrimidin-1 (2H)-yl)-1,3-dioxoisoindolin-5-yl)piperidin-4-yl)methyl)piperidin-4-yl)piperazin-1-yl)-5-(ethyl(tetrahydro-2H-pyran-4-yl)amino)-4-methyl-[1,1′-biphenyl]-3-carboxamide;
N-((4,6-dimethyl-2-oxo-1,2-dihydropyridin-3-yl)methyl)-4′-(4-(1-((1-(2-(2,4-dioxotetrahydropyrimidin-1 (2H)-yl)-1,3-dioxoisoindolin-4-yl)piperidin-4-yl)methyl)piperidin-4-yl)piperazin-1-yl)-5-(ethyl(tetrahydro-2H-pyran-4-yl)amino)-4-methyl-[1,1′-biphenyl]-3-carboxamide;
(R)-N-(1-(4-(4-((1-(2-(2,4-dioxotetrahydropyrimidin-1 (2H)-yl)-1,3-dioxoisoindolin-5-yl)piperidin-4-yl)methyl)piperazin-1-yl)benzyl)-1H-pyrazol-4-yl)-9-methyl-6-oxo-6,7,8,9-tetrahydropyrido[3′,2′: 4,5]pyrrolo[1,2-alpyrazine-2-carboxamide;
(R)-N-(1-(4-(4-((1-(2-(2,4-dioxotetrahydropyrimidin-1 (2H)-yl)-1,3-dioxoisoindolin-4-yl)piperidin-4-yl)methyl)piperazin-1-yl)benzyl)-1H-pyrazol-4-yl)-9-methyl-6-oxo-6,7,8,9-tetrahydropyrido[3′,2′: 4,5]pyrrolo[1,2-alpyrazine-2-carboxamide;
(R)-N-(1-(4-(4-(4-((1-(2-(2,4-dioxotetrahydropyrimidin-1 (2H)-yl)-1,3-dioxoisoindolin-5-yl)piperidin-4-yl)methyl)piperazin-1-yl)piperidin-1-yl)benzyl)-1H-pyrazol-4-yl)-9-methyl-6-oxo-6,7,8,9-tetrahydropyrido[3′,2′: 4,5]pyrrolo[1,2-a]pyrazine-2-carboxamide;
(R)-N-(1-(4-(4-(4-((1-(2-(2,4-dioxotetrahydropyrimidin-1 (2H)-yl)-1,3-dioxoisoindolin-4-yl)piperidin-4-yl)methyl)piperazin-1-yl)piperidin-1-yl)benzyl)-1H-pyrazol-4-yl)-9-methyl-6-oxo-6,7,8,9-tetrahydropyrido[3′,2′: 4,5]pyrrolo[1,2-a]pyrazine-2-carboxamide;
2-((5-bromo-2-((4-((4-((2-(2,4-dioxotetrahydropyrimidin-1 (2H)-yl)-1-oxoisoindolin-5-yl)methyl)piperazin-1-yl)sulfonyl)-2-methylphenyl)amino)pyrimidin-4-yl)amino)-6-fluorobenzamide;
2-((5-bromo-2-((4-((4-((2-(2,4-dioxotetrahydropyrimidin-1 (2H)-yl)-1,3-dioxoisoindolin-5-yl)methyl)piperazin-1-yl)sulfonyl)-2-methylphenyl)amino)pyrimidin-4-yl)amino)-6-fluorobenzamide;
2-((5-bromo-2-((4-((4-((1-(2-(2,4-dioxotetrahydropyrimidin-1 (2H)-yl)-1,3-dioxoisoindolin-5-yl)piperidin-4-yl)methyl)piperazin-1-yl)sulfonyl)-2-methylphenyl)amino)pyrimidin-4-yl)amino)-6-fluorobenzamide;
2-((5-bromo-2-((4-((4-((1-(2-(2,4-dioxotetrahydropyrimidin-1 (2H)-yl)-1,3-dioxoisoindolin-4-yl)piperidin-4-yl)methyl)piperazin-1-yl)sulfonyl)-2-methylphenyl)amino)pyrimidin-4-yl)amino)-6-fluorobenzamide;
1-(5-((4-((4-((5-amino-1-(2,6-difluorobenzoyl)-1H-1,2,4-triazol-3-yl)amino)phenyl)sulfonyl) piperazin-1-yl)methyl)-1-oxoisoindolin-2-yl)dihydropyrimidine-2,4 (1H,3H)-dione;
5-((4-((4-((5-amino-1-(2,6-difluorobenzoyl)-1H-1,2,4-triazol-3-yl)amino)phenyl)sulfonyl) piperazin-1-yl)methyl)-2-(2,4-dioxotetrahydropyrimidin-1 (2H)-yl)isoindoline-1,3-dione;
5-(4-((4-((4-((5-amino-1-(2,6-difluorobenzoyl)-1H-1,2,4-triazol-3-yl)amino)phenyl)sulfonyl) piperazin-1-yl)methyl)piperidin-1-yl)-2-(2,4-dioxotetrahydropyrimidin-1 (2H)-yl)isoindoline-1,3-dione;
4-(4-((4-((4-((5-amino-1-(2,6-difluorobenzoyl)-1H-1,2,4-triazol-3-yl)amino)phenyl)sulfonyl) piperazin-1-yl)methyl)piperidin-1-yl)-2-(2,4-dioxotetrahydropyrimidin-1 (2H)-yl)isoindoline-1,3-dione;
1-(5-((4-((4-((5-amino-1-(3-methylthiophene-2-carbonyl)-1H-1,2,4-triazol-3-yl)amino)phenyl)sulfonyl) piperazin-1-yl)methyl)-1-oxoisoindolin-2-yl)dihydropyrimidine-2,4 (1H,3H)-dione;
5-((4-((4-((5-amino-1-(3-methylthiophene-2-carbonyl)-1H-1,2,4-triazol-3-yl)amino)phenyl)sulfonyl) piperazin-1-yl)methyl)-2-(2,4-dioxotetrahydropyrimidin-1 (2H)-yl)isoindoline-1,3-dione
5-(4-((4-((4-((5-amino-1-(3-methylthiophene-2-carbonyl)-1H-1,2,4-triazol-3-yl)amino)phenyl)sulfonyl) piperazin-1-yl)methyl)piperidin-1-yl)-2-(2,4-dioxotetrahydropyrimidin-1 (2H)-yl)isoindoline-1,3-dione;
4-(4-((4-((4-((5-amino-1-(3-methylthiophene-2-carbonyl)-1H-1,2,4-triazol-3-yl)amino)phenyl)sulfonyl) piperazin-1-yl)methyl)piperidin-1-yl)-2-(2,4-dioxotetrahydropyrimidin-1 (2H)-yl)isoindoline-1,3-dione;
4-((7H-pyrrolo[2,3-d]pyrimidin-4-yl)amino)-N-(4-(4-(4-((2-(2,4-dioxotetrahydropyrimidin-1 (2H)-yl)-1-oxoisoindolin-5-yl)methyl)piperazin-1-yl)piperidin-1-yl)phenyl)-1H-pyrazole-3-carboxamide;
4-((7H-pyrrolo[2,3-d]pyrimidin-4-yl)amino)-N-(4-(4-(4-((2-(2,4-dioxotetrahydropyrimidin-1 (2H)-yl)-1,3-dioxoisoindolin-5-yl)methyl)piperazin-1-yl)piperidin-1-yl)phenyl)-1H-pyrazole-3-carboxamide;
4-((7H-pyrrolo[2,3-d]pyrimidin-4-yl)amino)-N-(4-(4-(4-((1-(2-(2,4-dioxotetrahydropyrimidin-1 (2H)-yl)-1,3-dioxoisoindolin-5-yl)piperidin-4-yl)methyl)piperazin-1-yl)piperidin-1-yl)phenyl)-1H-pyrazole-3-carboxamide;
4-((7H-pyrrolo[2,3-d]pyrimidin-4-yl)amino)-N-(4-(4-(4-((1-(2-(2,4-dioxotetrahydropyrimidin-1 (2H)-yl)-1,3-dioxoisoindolin-4-yl)piperidin-4-yl)methyl)piperazin-1-yl)piperidin-1-yl)phenyl)-1H-pyrazole-3-carboxamide;
1-(5-((4-(5-(5H-pyrido[4,3-b]indol-7-yl)pyridin-2-yl)piperazin-1-yl)methyl)-1-oxoisoindolin-2-yl)dihydropyrimidine-2,4 (1H,3H)-dione;
5-((4-(5-(5H-pyrido[4,3-b]indol-7-yl)pyridin-2-yl)piperazin-1-yl)methyl)-2-(2,4-dioxotetrahydropyrimidin-1 (2H)-yl)isoindoline-1,3-dione;
4-(4-((4-(5-(5H-pyrido[4,3-b]indol-7-yl)pyridin-2-yl)piperazin-1-yl)methyl)piperidin-1-yl)-2-(2,4-dioxotetrahydropyrimidin-1 (2H)-yl)isoindoline-1,3-dione;
1-(5-((4-(5-(5-methyl-5H-pyrido[4,3-b]indol-7-yl)pyridin-2-yl)piperazin-1-yl)methyl)-1-oxoisoindolin-2-yl)dihydropyrimidine-2,4 (1H,3H)-dione;
2-(2,4-dioxotetrahydropyrimidin-1 (2H)-yl)-5-((4-(5-(5-methyl-5H-pyrido[4,3-b]indol-7-yl)pyridin-2-yl) piperazin-1-yl)methyl)isoindoline-1,3-dione;
2-(2,4-dioxotetrahydropyrimidin-1 (2H)-yl)-5-(4-((4-(5-(5-methyl-5H-pyrido[4,3-b]indol-7-yl)pyridin-2-yl) piperazin-1-yl)methyl)piperidin-1-yl)isoindoline-1,3-dione;
2-(2,4-dioxotetrahydropyrimidin-1 (2H)-yl)-4-(4-((4-(5-(5-methyl-5H-pyrido[4,3-b]indol-7-yl)pyridin-2-yl) piperazin-1-yl)methyl)piperidin-1-yl)isoindoline-1,3-dione;
1-(1-oxo-5-((4-(5-(5-(2,2,2-trifluoroethyl)-5H-pyrido[4,3-b]indol-7-yl)pyridin-2-yl)piperazin-1-yl)methyl)isoindolin-2-yl)dihydropyrimidine-2,4 (1H,3H)-dione;
2-(2,4-dioxotetrahydropyrimidin-1 (2H)-yl)-5-((4-(5-(5-(2,2,2-trifluoroethyl)-5H-pyrido[4,3-b]indol-7-yl)pyridin-2-yl)piperazin-1-yl)methyl)isoindoline-1,3-dione;
2-(2,4-dioxotetrahydropyrimidin-1 (2H)-yl)-5-(4-((4-(5-(5-(2,2,2-trifluoroethyl)-5H-pyrido[4,3-b]indol-7-yl)pyridin-2-yl)piperazin-1-yl)methyl)piperidin-1-yl)isoindoline-1,3-dione;
2-(2,4-dioxotetrahydropyrimidin-1 (2H)-yl)-4-(4-((4-(5-(5-(2,2,2-trifluoroethyl)-5H-pyrido[4,3-b]indol-7-yl)pyridin-2-yl)piperazin-1-yl)methyl)piperidin-1-yl)isoindoline-1,3-dione;
1-(5-((4-(5-(5-(difluoromethyl)-5H-pyrido[4,3-b]indol-7-yl)pyridin-2-yl)piperazin-1-yl)methyl)-1-oxoisoindolin-2-yl)dihydropyrimidine-2,4 (1H,3H)-dione;
5-((4-(5-(5-(difluoromethyl)-5H-pyrido[4,3-b]indol-7-yl)pyridin-2-yl)piperazin-1-yl)methyl)-2-(2,4-dioxotetrahydropyrimidin-1 (2H)-yl)isoindoline-1,3-dione;
5-(4-((4-(5-(5-(difluoromethyl)-5H-pyrido[4,3-b]indol-7-yl)pyridin-2-yl)piperazin-1-yl)methyl)piperidin-1-yl)-2-(2,4-dioxotetrahydropyrimidin-1 (2H)-yl)isoindoline-1,3-dione;
4-(4-((4-(5-(5-(difluoromethyl)-5H-pyrido[4,3-b]indol-7-yl)pyridin-2-yl)piperazin-1-yl)methyl)piperidin-1-yl)-2-(2,4-dioxotetrahydropyrimidin-1 (2H)-yl)isoindoline-1,3-dione;
1-(5-((4-(5-(5-methyl-5H-pyrido[4,3-b]indol-7-yl)-3-(trifluoromethyl)pyridin-2-yl)piperazin-1-yl)methyl)-1-oxoisoindolin-2-yl)dihydropyrimidine-2,4 (1H,3H)-dione;
2-(2,4-dioxotetrahydropyrimidin-1 (2H)-yl)-5-((4-(5-(5-methyl-5H-pyrido[4,3-b]indol-7-yl)-3-(trifluoromethyl)pyridin-2-yl)piperazin-1-yl)methyl)isoindoline-1,3-dione;
2-(2,4-dioxotetrahydropyrimidin-1 (2H)-yl)-5-(4-((4-(5-(5-methyl-5H-pyrido[4,3-b]indol-7-yl)-3-(trifluoromethyl)pyridin-2-yl)piperazin-1-yl)methyl)piperidin-1-yl)isoindoline-1,3-dione;
2-(2,4-dioxotetrahydropyrimidin-1 (2H)-yl)-4-(4-((4-(5-(5-methyl-5H-pyrido[4,3-b]indol-7-yl)-3-(trifluoromethyl)pyridin-2-yl)piperazin-1-yl)methyl)piperidin-1-yl)isoindoline-1,3-dione;
2-(2,4-dioxotetrahydropyrimidin-1 (2H)-yl)-5-((4-(5-(5-methyl-5H-pyrido[4,3-b]indol-7-yl)-4-(trifluoromethyl)pyridin-2-yl)piperazin-1-yl)methyl)isoindoline-1,3-dione;
2-(2,4-dioxotetrahydropyrimidin-1 (2H)-yl)-5-(4-((4-(5-(5-methyl-5H-pyrido[4,3-b]indol-7-yl)-4-(trifluoromethyl)pyridin-2-yl)piperazin-1-yl)methyl)piperidin-1-yl)isoindoline-1,3-dione;
2-(2,4-dioxotetrahydropyrimidin-1 (2H)-yl)-4-(4-((4-(5-(5-methyl-5H-pyrido[4,3-b]indol-7-yl)-4-(trifluoromethyl)pyridin-2-yl)piperazin-1-yl)methyl)piperidin-1-yl)isoindoline-1,3-dione;
1-(5-((4-(3-methyl-5-(5-methyl-5H-pyrido[4,3-b]indol-7-yl)pyridin-2-yl)piperazin-1-yl)methyl)-1-oxoisoindolin-2-yl)dihydropyrimidine-2,4 (1H,3H)-dione;
2-(2,4-dioxotetrahydropyrimidin-1 (2H)-yl)-5-((4-(3-methyl-5-(5-methyl-5H-pyrido[4,3-b]indol-7-yl)pyridin-2-yl)piperazin-1-yl)methyl)isoindoline-1,3-dione;
2-(2,4-dioxotetrahydropyrimidin-1 (2H)-yl)-5-(4-((4-(3-methyl-5-(5-methyl-5H-pyrido[4,3-b]indol-7-yl)pyridin-2-yl)piperazin-1-yl)methyl)piperidin-1-yl)isoindoline-1,3-dione;
2-(2,4-dioxotetrahydropyrimidin-1 (2H)-yl)-4-(4-((4-(3-methyl-5-(5-methyl-5H-pyrido[4,3-b]indol-7-yl)pyridin-2-yl)piperazin-1-yl)methyl)piperidin-1-yl)isoindoline-1,3-dione;
1-(5-((4-(3-chloro-5-(5-methyl-5H-pyrido[4,3-b]indol-7-yl)pyridin-2-yl)piperazin-1-yl)methyl)-1-oxoisoindolin-2-yl)dihydropyrimidine-2,4 (1H,3H)-dione;
5-((4-(3-chloro-5-(5-methyl-5H-pyrido[4,3-b]indol-7-yl)pyridin-2-yl)piperazin-1-yl)methyl)-2-(2,4-dioxotetrahydropyrimidin-1 (2H)-yl)isoindoline-1,3-dione;
5-(4-((4-(3-chloro-5-(5-methyl-5H-pyrido[4,3-b]indol-7-yl)pyridin-2-yl)piperazin-1-yl)methyl)piperidin-1-yl)-2-(2,4-dioxotetrahydropyrimidin-1 (2H)-yl)isoindoline-1,3-dione;
4-(4-((4-(3-chloro-5-(5-methyl-5H-pyrido[4,3-b]indol-7-yl)pyridin-2-yl)piperazin-1-yl)methyl)piperidin-1-yl)-2-(2,4-dioxotetrahydropyrimidin-1 (2H)-yl)isoindoline-1,3-dione;
1-(5-((4-(benzo[4,5]imidazo[1,2-a]pyrimidin-2-yl)piperazin-1-yl)methyl)-1-oxoisoindolin-2-yl)dihydropyrimidine-2,4 (1H,3H)-dione;
5-((4-(benzo[4,5]imidazo[1,2-a]pyrimidin-2-yl)piperazin-1-yl)methyl)-2-(2,4-dioxotetrahydropyrimidin-1 (2H)-yl)isoindoline-1,3-dione;
5-(4-((4-(benzo[4,5]imidazo[1,2-a]pyrimidin-2-yl)piperazin-1-yl)methyl)piperidin-1-yl)-2-(2,4-dioxotetrahydropyrimidin-1 (2H)-yl)isoindoline-1,3-dione;
4-(4-((4-(benzo[4,5]imidazo[1,2-a]pyrimidin-2-yl)piperazin-1-yl)methyl)piperidin-1-yl)-2-(2,4-dioxotetrahydropyrimidin-1 (2H)-yl)isoindoline-1,3-dione;
5-((4-(1-(benzo[4,5]imidazo[1,2-a]pyrimidin-2-yl)piperidin-4-yl)piperazin-1-yl)methyl)-2-(2,4-dioxotetrahydropyrimidin-1 (2H)-yl)isoindoline-1,3-dione;
1-(5-(((1-(4-(4-acetyl-1,4-diazepan-1-yl)-6-((2-(thiophen-2-yl)ethyl)amino)-1,3,5-triazin-2-yl)piperidin-4-yl)(methyl)amino)methyl)-1-oxoisoindolin-2-yl)dihydropyrimidine-2,4 (1H,3H)-dione;
5-(((1-(4-(4-acetyl-1,4-diazepan-1-yl)-6-((2-(thiophen-2-yl)ethyl)amino)-1,3,5-triazin-2-yl)piperidin-4-yl)(methyl)amino)methyl)-2-(2,4-dioxotetrahydropyrimidin-1 (2H)-yl)isoindoline-1,3-dione;
5-(4-((1-(benzo[4,5]imidazo[1,2-a]pyrimidin-2-yl)piperidin-4-yl)methyl)piperazin-1-yl)-2-(2,4-dioxotetrahydropyrimidin-1 (2H)-yl)isoindoline-1,3-dione;
5-((1S,4S)-5-((1-(benzo[4,5]imidazo[1,2-a]pyrimidin-2-yl)piperidin-4-yl)methyl)-2,5-diazabicyclo[2.2.1]heptan-2-yl)-2-(2,4-dioxotetrahydropyrimidin-1 (2H)-yl)isoindoline-1,3-dione;
5-((1R,4R)-5-((1-(benzo[4,5]imidazo[1,2-a]pyrimidin-2-yl)piperidin-4-yl)methyl)-2,5-diazabicyclo[2.2.1]heptan-2-yl)-2-(2,4-dioxotetrahydropyrimidin-1 (2H)-yl)isoindoline-1,3-dione;
5-(4-((1-(benzo[4,5]imidazo[1,2-a]pyrimidin-2-yl)piperidin-4-yl)methyl)piperazin-1-yl)-2-(2,4-dioxotetrahydropyrimidin-1 (2H)-yl)-6-fluoroisoindoline-1,3-dione;
5-(3-((1-(benzo[4,5]imidazo[1,2-a]pyrimidin-2-yl)piperidin-4-yl)methyl)-3,8-diazabicyclo[3.2.1]octan-8-yl)-2-(2,4-dioxotetrahydropyrimidin-1 (2H)-yl)isoindoline-1,3-dione;
5-(6-((1-(benzo[4,5]imidazo[1,2-a]pyrimidin-2-yl)piperidin-4-yl)methyl)-3,6-diazabicyclo[3.1.1]heptan-3-yl)-2-(2,4-dioxotetrahydropyrimidin-1 (2H)-yl)isoindoline-1,3-dione;
5-(3-((1-(benzo[4,5]imidazo[1,2-a]pyrimidin-2-yl)piperidin-4-yl)methyl)-3,6-diazabicyclo[3.1.1]heptan-6-yl)-2-(2,4-dioxotetrahydropyrimidin-1 (2H)-yl)isoindoline-1,3-dione;
5-(8-((1-(benzo[4,5]imidazo[1,2-a]pyrimidin-2-yl)piperidin-4-yl)methyl)-3,8-diazabicyclo[3.2.1]octan-3-yl)-2-(2,4-dioxotetrahydropyrimidin-1 (2H)-yl)isoindoline-1,3-dione;
4-(4-((1-(benzo[4,5]imidazo[1,2-a]pyrimidin-2-yl)piperidin-4-yl)methyl)piperazin-1-yl)-2-(2,4-dioxotetrahydropyrimidin-1 (2H)-yl)isoindoline-1,3-dione;
5-(4-((1-(benzo[4,5]imidazo[1,2-a]pyrimidin-2-yl)piperidin-4-yl)methyl)piperazin-1-yl)-2-(2,4-dioxotetrahydropyrimidin-1 (2H)-yl)-4-fluoroisoindoline-1,3-dione;
6-(4-((1-(benzo[4,5]imidazo[1,2-a]pyrimidin-2-yl)piperidin-4-yl)methyl)piperazin-1-yl)-2-(2,4-dioxotetrahydropyrimidin-1 (2H)-yl)-4-fluoroisoindoline-1,3-dione;
5-(4-(4-((1-(benzo[4,5]imidazo[1,2-a]pyrimidin-2-yl)piperidin-4-yl)methyl)piperazin-1-yl)piperidin-1-yl)-2-(2,4-dioxotetrahydropyrimidin-1 (2H)-yl)isoindoline-1,3-dione;
5-(4-(4-((1-(benzo[4,5]imidazo[1,2-a]pyrimidin-2-yl)piperidin-4-yl)methyl)piperazin-1-yl)-3,3-difluoropiperidin-1-yl)-2-(2,4-dioxotetrahydropyrimidin-1 (2H)-yl)isoindoline-1,3-dione;
5-(5-((1-(benzo[4,5]imidazo[1,2-a]pyrimidin-2-yl)piperidin-4-yl)methyl)-2,5-diazabicyclo[2.2.2]octan-2-yl)-2-(2,4-dioxotetrahydropyrimidin-1 (2H)-yl)isoindoline-1,3-dione;
1-(5-((4-chloro-2-(7-chloro-5-(trifluoromethyl)-1H-benzo[d]imidazol-2-yl)phenoxy)methyl)-1-oxoisoindolin-2-yl)dihydropyrimidine-2,4 (1H,3H)-dione;
5-((4-chloro-2-(7-chloro-5-(trifluoromethyl)-1H-benzo[d]imidazol-2-yl)phenoxy)methyl)-2-(2,4-dioxotetrahydropyrimidin-1 (2H)-yl)isoindoline-1,3-dione;
1-(4-((5-(3,5-dimethylisoxazol-4-yl)-2-methylphenyl)((1-((2-(2,4-dioxotetrahydropyrimidin-1 (2H)-yl)-1-oxoisoindolin-5-yl)methyl)piperidin-4-yl)methyl)amino)phenyl)cyclopropane-1-carbonitrile;
1-(4-((5-(3,5-dimethylisoxazol-4-yl)-2-methylphenyl)((1-((2-(2,4-dioxotetrahydropyrimidin-1 (2H)-yl)-1,3-dioxoisoindolin-5-yl)methyl)piperidin-4-yl)methyl)amino)phenyl)cyclopropane-1-carbonitrile;
1-(4-((5-(3,5-dimethylisoxazol-4-yl)-2-methylphenyl)((1-((1-(2-(2,4-dioxotetrahydropyrimidin-1 (2H)-yl)-1,3-dioxoisoindolin-5-yl)piperidin-4-yl)methyl)piperidin-4-yl)methyl)amino)phenyl)cyclopropane-1-carbonitrile;
1-(4-((5-(3,5-dimethylisoxazol-4-yl)-2-methylphenyl)((1-((1-(2-(2,4-dioxotetrahydropyrimidin-1 (2H)-yl)-1,3-dioxoisoindolin-4-yl)piperidin-4-yl)methyl)piperidin-4-yl)methyl)amino)phenyl)cyclopropane-1-carbonitrile;
1-(5-((2-(6-methyl-7-oxo-6,7-dihydro-1H-pyrrolo[2,3-c]pyridin-4-yl)-4-(methylsulfonyl)phenoxy)methyl)-1-oxoisoindolin-2-yl)dihydropyrimidine-2,4 (1H,3H)-dione;
N-((1r,4r)-4-(3-chloro-4-cyanophenoxy)cyclohexyl)-6-(4-((3-(2-(2,4-dioxotetrahydropyrimidin-1 (2H)-yl)-1,3-dioxoisoindolin-5-yl)-3,6-diazabicyclo[3.1.1]heptan-6-yl)methyl)piperidin-1-yl)pyridazine-3-carboxamide;
N-((1r,4r)-4-(3-chloro-4-cyanophenoxy)cyclohexyl)-6-(4-((3-(2-(2,4-dioxotetrahydropyrimidin-1 (2H)-yl)-1,3-dioxoisoindolin-5-yl)-3,8-diazabicyclo[3.2.1]octan-8-yl)methyl)piperidin-1-yl)pyridazine-3-carboxamide;
N-((1r,4r)-4-(3-chloro-4-cyanophenoxy)cyclohexyl)-6-(4-((4-(1-(2-(2,4-dioxotetrahydropyrimidin-1 (2H)-yl)-1,3-dioxoisoindolin-5-yl)piperidin-4-yl)piperazin-1-yl)methyl)piperidin-1-yl)pyridazine-3-carboxamide;
N-((1r,4r)-4-(3-chloro-4-cyanophenoxy)cyclohexyl)-6-(4-((5-(2-(2,4-dioxotetrahydropyrimidin-1 (2H)-yl)-1,3-dioxoisoindolin-5-yl)-2,5-diazabicyclo[2.2.2]octan-2-yl)methyl)piperidin-1-yl)pyridazine-3-carboxamide;
N-((1r,3r)-3-(3-chloro-4-cyanophenoxy)-2,2,4,4-tetramethylcyclobutyl)-6-(4-((3-(2-(2,4-dioxotetrahydropyrimidin-1 (2H)-yl)-1,3-dioxoisoindolin-5-yl)-3,6-diazabicyclo[3.1.1]heptan-6-yl)methyl)piperidin-1-yl) nicotinamide;
N-((1r,3r)-3-(3-chloro-4-cyanophenoxy)-2,2,4,4-tetramethylcyclobutyl)-6-(4-((3-(2-(2,4-dioxotetrahydropyrimidin-1 (2H)-yl)-1,3-dioxoisoindolin-5-yl)-3,8-diazabicyclo[3.2.1]octan-8-yl)methyl)piperidin-1-yl) nicotinamide;
N-((1r,3r)-3-(3-chloro-4-cyanophenoxy)-2,2,4,4-tetramethylcyclobutyl)-6-(4-((4-(2-(2,4-dioxotetrahydropyrimidin-1 (2H)-yl)-4-fluoro-1,3-dioxoisoindolin-5-yl)piperazin-1-yl)methyl)piperidin-1-yl) nicotinamide;
N-((1r,3r)-3-(3-chloro-4-cyanophenoxy)-2,2,4,4-tetramethylcyclobutyl)-6-(4-((4-(1-(2-(2,4-dioxotetrahydropyrimidin-1 (2H)-yl)-1,3-dioxoisoindolin-5-yl)piperidin-4-yl)piperazin-1-yl)methyl)piperidin-1-yl) nicotinamide;
N-((1r,3r)-3-(3-chloro-4-cyanophenoxy)-2,2,4,4-tetramethylcyclobutyl)-6-(4-((5-(2-(2,4-dioxotetrahydropyrimidin-1 (2H)-yl)-1,3-dioxoisoindolin-5-yl)-2,5-diazabicyclo[2.2.2]octan-2-yl)methyl)piperidin-1-yl) nicotinamide;
(S)-5-chloro-4-(4-chloro-1-methyl-1H-pyrazol-5-yl)-N-(1-(((2-(2,4-dioxotetrahydropyrimidin-1 (2H)-yl)-1-oxoisoindolin-5-yl)methyl)amino)-3-(3-fluorophenyl) propan-2-yl)thiophene-2-carboxamide;
1-(5-(((1-(4-(2-(2-aminopyridin-3-yl)-5-phenyl-3H-imidazo[4,5-b]pyridin-3-yl)phenyl)cyclobutyl)amino)methyl)-1-oxoisoindolin-2-yl)dihydropyrimidine-2,4 (1H,3H)-dione;
(S)-5-chloro-4-(4-chloro-1-methyl-1H-pyrazol-5-yl)-N-(1-(3,4-difluorophenyl)-3-(((2-(2,4-dioxotetrahydropyrimidin-1 (2H)-yl)-1-oxoisoindolin-5-yl)methyl)amino) propan-2-yl) furan-2-carboxamide;
(S)-5-chloro-4-(4-chloro-1-methyl-1H-pyrazol-5-yl)-N-(1-(3,4-difluorophenyl)-3-(4-((2-(2,4-dioxotetrahydropyrimidin-1 (2H)-yl)-1-oxoisoindolin-5-yl)methyl)piperazin-1-yl) propan-2-yl) furan-2-carboxamide;
(S)-5-chloro-4-(4-chloro-1-methyl-1H-pyrazol-5-yl)-N-(1-(4-((2-(2,4-dioxotetrahydropyrimidin-1 (2H)-yl)-1-oxoisoindolin-5-yl)methyl)piperazin-1-yl)-3-(3-fluorophenyl) propan-2-yl)thiophene-2-carboxamide;
(S)-5-chloro-4-(4-chloro-1-methyl-1H-pyrazol-5-yl)-N-(1-(((2-(2,4-dioxotetrahydropyrimidin-1 (2H)-yl)-1,3-dioxoisoindolin-5-yl)methyl)amino)-3-(3-fluorophenyl) propan-2-yl)thiophene-2-carboxamide;
5-(((1-(4-(2-(2-aminopyridin-3-yl)-5-phenyl-3H-imidazo[4,5-b]pyridin-3-yl)phenyl)cyclobutyl)amino)methyl)-2-(2,4-dioxotetrahydropyrimidin-1 (2H)-yl)isoindoline-1,3-dione;
(S)-5-chloro-4-(4-chloro-1-methyl-1H-pyrazol-5-yl)-N-(1-(3,4-difluorophenyl)-3-(((2-(2,4-dioxotetrahydropyrimidin-1 (2H)-yl)-1,3-dioxoisoindolin-5-yl)methyl)amino) propan-2-yl) furan-2-carboxamide;
(S)-5-chloro-4-(4-chloro-1-methyl-1H-pyrazol-5-yl)-N-(1-(3,4-difluorophenyl)-3-(4-((2-(2,4-dioxotetrahydropyrimidin-1 (2H)-yl)-1,3-dioxoisoindolin-5-yl)methyl)piperazin-1-yl) propan-2-yl) furan-2-carboxamide;
(S)-5-chloro-4-(4-chloro-1-methyl-1H-pyrazol-5-yl)-N-(1-(4-((2-(2,4-dioxotetrahydropyrimidin-1 (2H)-yl)-1,3-dioxoisoindolin-5-yl)methyl)piperazin-1-yl)-3-(3-fluorophenyl) propan-2-yl)thiophene-2-carboxamide;
(S)-5-chloro-4-(4-chloro-1-methyl-1H-pyrazol-5-yl)-N-(1-(((1-(2-(2,4-dioxotetrahydropyrimidin-1 (2H)-yl)-1,3-dioxoisoindolin-5-yl)piperidin-4-yl)methyl)amino)-3-(3-fluorophenyl) propan-2-yl)thiophene-2-carboxamide;
5-(4-(((1-(4-(2-(2-aminopyridin-3-yl)-5-phenyl-3H-imidazo[4,5-b]pyridin-3-yl)phenyl)cyclobutyl)amino)methyl)piperidin-1-yl)-2-(2,4-dioxotetrahydropyrimidin-1 (2H)-yl)isoindoline-1,3-dione;
(S)-5-chloro-4-(4-chloro-1-methyl-1H-pyrazol-5-yl)-N-(1-(3,4-difluorophenyl)-3-(((1-(2-(2,4-dioxotetrahydropyrimidin-1 (2H)-yl)-1,3-dioxoisoindolin-5-yl)piperidin-4-yl)methyl)amino) propan-2-yl) furan-2-carboxamide;
(S)-5-chloro-4-(4-chloro-1-methyl-1H-pyrazol-5-yl)-N-(1-(3,4-difluorophenyl)-3-(4-((1-(2-(2,4-dioxotetrahydropyrimidin-1 (2H)-yl)-1,3-dioxoisoindolin-5-yl)piperidin-4-yl)methyl)piperazin-1-yl) propan-2-yl) furan-2-carboxamide;
(S)-5-chloro-4-(4-chloro-1-methyl-1H-pyrazol-5-yl)-N-(1-(4-((1-(2-(2,4-dioxotetrahydropyrimidin-1 (2H)-yl)-1,3-dioxoisoindolin-5-yl)piperidin-4-yl)methyl)piperazin-1-yl)-3-(3-fluorophenyl) propan-2-yl)thiophene-2-carboxamide;
(S)-5-chloro-4-(4-chloro-1-methyl-1H-pyrazol-5-yl)-N-(1-(((1-(2-(2,4-dioxotetrahydropyrimidin-1 (2H)-yl)-1,3-dioxoisoindolin-4-yl)piperidin-4-yl)methyl)amino)-3-(3-fluorophenyl) propan-2-yl)thiophene-2-carboxamide;
4-(4-(((1-(4-(2-(2-aminopyridin-3-yl)-5-phenyl-3H-imidazo[4,5-b]pyridin-3-yl)phenyl)cyclobutyl)amino)methyl)piperidin-1-yl)-2-(2,4-dioxotetrahydropyrimidin-1 (2H)-yl)isoindoline-1,3-dione;
(S)-5-chloro-4-(4-chloro-1-methyl-1H-pyrazol-5-yl)-N-(1-(3,4-difluorophenyl)-3-(((1-(2-(2,4-dioxotetrahydropyrimidin-1 (2H)-yl)-1,3-dioxoisoindolin-4-yl)piperidin-4-yl)methyl)amino) propan-2-yl) furan-2-carboxamide;
(S)-5-chloro-4-(4-chloro-1-methyl-1H-pyrazol-5-yl)-N-(1-(3,4-difluorophenyl)-3-(4-((1-(2-(2,4-dioxotetrahydropyrimidin-1 (2H)-yl)-1,3-dioxoisoindolin-4-yl)piperidin-4-yl)methyl)piperazin-1-yl) propan-2-yl) furan-2-carboxamide;
(S)-5-chloro-4-(4-chloro-1-methyl-1H-pyrazol-5-yl)-N-(1-(4-((1-(2-(2,4-dioxotetrahydropyrimidin-1 (2H)-yl)-1,3-dioxoisoindolin-4-yl)piperidin-4-yl)methyl)piperazin-1-yl)-3-(3-fluorophenyl) propan-2-yl)thiophene-2-carboxamide;
1-(5-(((4-(8-fluoro-1-oxo-2,3,4,6-tetrahydro-1H-azepino[5,4,3-cd]indol-5-yl)benzyl)(methyl)amino)methyl)-1-oxoisoindolin-2-yl)dihydropyrimidine-2,4 (1H,3H)-dione;
2-(2,4-dioxotetrahydropyrimidin-1 (2H)-yl)-5-(((4-(8-fluoro-1-oxo-2,3,4,6-tetrahydro-1H-azepino[5,4,3-cd]indol-5-yl)benzyl)(methyl)amino)methyl)isoindoline-1,3-dione;
2-(2,4-dioxotetrahydropyrimidin-1 (2H)-yl)-5-(4-(((4-(8-fluoro-1-oxo-2,3,4,6-tetrahydro-1H-azepino[5,4,3-cd]indol-5-yl)benzyl)(methyl)amino)methyl)piperidin-1-yl)isoindoline-1,3-dione;
2-(2,4-dioxotetrahydropyrimidin-1 (2H)-yl)-4-(4-(((4-(8-fluoro-1-oxo-2,3,4,6-tetrahydro-1H-azepino[5,4,3-cd]indol-5-yl)benzyl)(methyl)amino)methyl)piperidin-1-yl)isoindoline-1,3-dione; and
1-(5-((4-(1-(benzo[4,5]imidazo[1,2-a]pyrimidin-2-yl)piperidin-4-yl)piperazin-1-yl)methyl)-1-oxoisoindolin-2-yl)dihydropyrimidine-2,4 (1H,3H)-dione.

20. The compound of Formula (I) or salts, enantiomers, stereoisomers, solvates, or polymorphs thereof as claimed in claim 1, which is a hydrohalic acid salt (including hydrochloride, hydrobromide), sulfate, citrate, maleate, mesylate, lactate, lactobionate, L-tartrate, fumarate, L-malate, α-ketoglutarate, hippurate, D-glucuronate, D-gluconate, α-D-glucoheptonate, glycolate, mucate, L-ascorbate, orotate, picrate, glycinate, alaninate, arginate, cinnamate, laurate, pamoate, sebacate, besylate, methanesulfonate, ethanesulfonate, edisylate, formate, acetate, 2,2-dichloroacetate, trimethylacetate, propionate, valerate, palmitate, triphenylacetate, 2-ethyl-butane-1,4-dioate, iodate, nicotinate, L-pyroglutamate, L-prolinate, ferulate, 2-hydroxyethanesulfonate, nitrate, gentisate, cholate, salicylate, terephthalate, glutarate, adipate, stearate, oleate, undecylenate, camphorate, camsylate, dodecylsulfate, phosphate, thiocyanate, dihydrogen phosphate, pyrophosphate, metaphosphate, oxalate, malonate, benzoate, mandelate, succinate, pyruvate, 4-chlorobenzenesulfonate, 1,5-naphthalenedisulfonate, 3-hydroxy-2-naphthoate, 1-hydroxy-2-naphthoate, 2-naphthalenesulfonate, trifluoroacetate, methanesulfonate, hippurate, glycolate, or 4-methylbenzenesulfonate of the compound of Formula (I).

21. A compound of Formula (II) 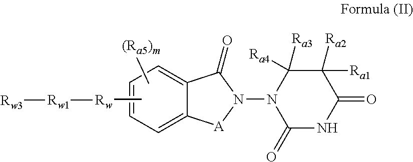
or salts, enantiomers, stereoisomers, solvates, isotopically enriched analogs, prodrugs, or polymorphs thereof,
wherein
A represents CO, CH₂, or CD₂;
Rₐ₁, Rₐ₂, Rₐ₃, and Rₐ₄ are the same or different and independently represent hydrogen, deuterium, halogen, C₁₋₆ alkyl, halogenated C₁₋₆ alkyl, deuterated C₁₋₆ alkyl, C₁₋₆ alkoxy, deuterated C₁₋₆ alkoxy, or halogenated C₁₋₆ alkoxy;
(Rₐ₅)ₘ indicates that the isoindoline ring to which it is attached is optionally substituted with m Rₐ₅, with each Rₐ₅ being the same or different and each independently representing halogen, hydroxy, mercapto, nitro, amino, cyano, C₁₋₆ alkyl, halogenated C₁₋₆ alkyl, deuterated C₁₋₆ alkyl, C₁₋₆ alkoxy, deuterated C₁₋₆ alkoxy, halogenated C₁₋₆ alkoxy, C₂₋₆ alkenyl, or C₂₋₆ alkynyl;
m represents an integer of 0, 1, 2, 3, or 4;
R_{w} represents O, S, S(O), S(O)₂, alkenylene, alkynylene, or N(Rₐ₆), wherein Rₐ₆ represents H or C₁₋₃ alkyl, or R_{w} represents a bond, or
R_{w} represents: 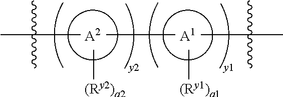
wherein each ring A¹ is the same or different and independently represents nitrogen-containing heterocyclylene, arylene, or heteroarylene, y1 represents an integer of 1 or 2, and (R^{y1})ₐ₁ indicates that each ring A¹ is independently optionally substituted with a1 R^{y1} substituents, wherein each R^{y1} independently represents deuterium, C₁₋₆ alkyl, deuterated C₁₋₆ alkyl, halogenated C₁₋₆ alkyl, C₁₋₆ alkoxy, halogen, amino, hydroxy, mercapto, cyano, carboxyl, C₂₋₆ alkynyl, or C₂₋₆ alkenyl, and a1 represents an integer of 0-20;
each ring A² is the same or different and independently represents heterocyclylene, cycloalkylene, arylene, or heteroarylene, y2 represents an integer of 0 or 1, and (R^{y2})ₐ₂ indicates that each ring A² is independently optionally substituted with a2 R^{y2} substituents, wherein each R^{y2} independently represents deuterium, C₁₋₆ alkyl, deuterated C₁₋₆ alkyl, halogenated C₁₋₆ alkyl, C₁₋₆ alkoxy, halogen, amino, hydroxy, mercapto, cyano, carboxyl, C₂₋₆ alkynyl, or C₂₋₆ alkenyl, and a2 represents an integer of 0-20; and
R_{w1} represents a bond or optionally substituted linear or branched C₁₋₂₀ alkylene, wherein any one or more methylene groups in the main carbon chain skeleton of the linear or branched C₁₋₂₀ alkylene are optionally replaced by one or more groups selected from Rₐ, R_{b}, and any combination thereof; wherein each R^{a} is independently selected from the group consisting of: O, C(O), OC(O), S, S(O), S(O)₂, S(O)₂N(Rₐ₇), N(Rₐ₇)S(O)₂, C(O)N(Rₐ₇), N(Rₐ₇)C(O), N(Rₐ₇), and N(Rₐ₇)C(O)N(Rₐ₇), where each Rₐ₇ is independently H or C₁₋₃ alkyl, and when one or more adjacent methylene groups in the main carbon chain skeleton of the linear or branched C₁₋₂₀ alkylene are replaced by one or more Rₐ groups, the Rₐ groups are not directly connected to each other; and wherein each R^{b} is independently selected from the group consisting of: optionally substituted cycloalkylene, optionally substituted arylene, optionally substituted heterocyclylene, optionally substituted heteroarylene, alkynylene, alkenylene, and any combination thereof, and
R_{w3} represents hydrogen, deuterium, leaving group, hydroxy, halogen, COOH, NH₂, N₃, CHO, methanesulfonyloxy, trifluoromethanesulfonyloxy or p-toluenesulfonyloxy.

22. The compound of Formula (II) or salts, enantiomers, stereoisomers, solvates, isotopically enriched analogs, prodrugs, or polymorphs thereof as claimed in claim 21, wherein the compound of Formula (II) is also represented by Formula (II-1) or Formula (II-2): 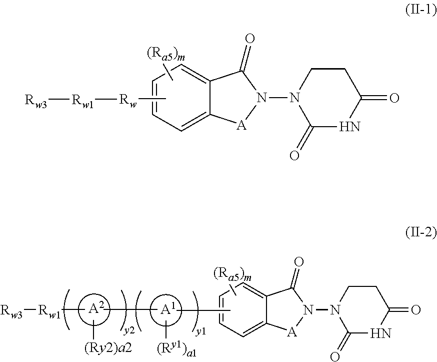
wherein A, (Rₐ₅)ₘ, R_{w}, ring A¹, ring A², y1, (R^{y1})ₐ₁, y2, (R^{y2})ₐ₂, R_{w1}, and R_{w3} are as defined in claim 21.

23. The compound of Formula (II) or salts, enantiomers, stereoisomers, solvates, isotopically enriched analogs, prodrugs, or polymorphs thereof as claimed in claim 21, wherein
(i) each ring A¹ independently represents 4-to 20-membered nitrogen-containing heterocyclylene, C₅₋₂₀ arylene, or 5-to 20-membered heteroarylene; preferably each ring A¹ independently represents:
piperidinylene, piperazinylene, morpholinylene, azetidinylene, pyrrolidinylene, imidazolidylene, pyrazolidylene, oxazolidinylene, thiazolidinylene, thiomorpholinylene, azepanylene, diazacycloheptanylene, azacyclooctylene, diazacyclooctylene, azabicyclo[3.1.1]heptanylene, azabicyclo[2.2.1]heptanylene, azabicyclo[3.2.1]octanylene, azabicyclo[2.2.2]octanylene, diazabicyclo[3.1.1]heptanylene, diazabicyclo[2.2.1]heptanylene, diazabicyclo[3.2.1]octanylene, diazabicyclo[2.2.2]octanylene, 3-azaspiro[5.5]undecanylene, 8-azaspiro[4.5]decanylene, 2-oxa-8-azaspiro[4.5]decanylene, 3-methyl-2-oxa-8-azaspiro[4.5]decanylene, phenylene, naphthylene, furanylene, oxazolylene, isoxazolylene, oxadiazolylene, thienylene, thiazolylene, isothiazolylene, thiadiazolylene, pyrrolylene, imidazolylene, pyrazolylene, triazolylene, pyridylene, pyrimidinylene, pyridazinylene, pyrazinylene, indolylene, isoindolylene, benzofuranylene, isobenzofuranylene, benzothienylene, indazolylene, benzimidazolylene, benzoxazolylene, benzisoxazolylene, benzothiazolylene, benzisothiazolylene, benzotriazolylene, benzo[2,1,3]oxadiazolylene, benzo[2,1,3]thiadiazolylene, benzo[1,2,3]thiadiazolylene, quinolinylene, isoquinolinylene, naphthyridinylene, cinnolinylene, quinazolinylene, quinoxalinylene, phthalazinylene, pyrazolo[1,5-a]pyridylene, pyrazolo[1,5-alpyrimidinylene, imidazo[1,2-a]pyridylene, 1H-pyrrolo[3,2-b]pyridylene, 1H-pyrrolo[2,3-b]pyridylene, pyrrolo[2,1-b]thiazolylene, imidazo[2,1-b]thiazolylene, or 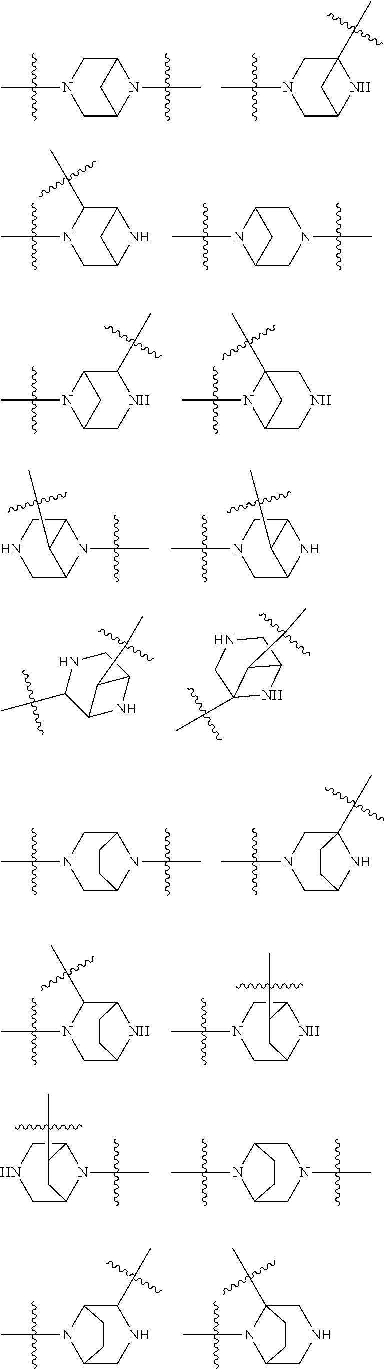 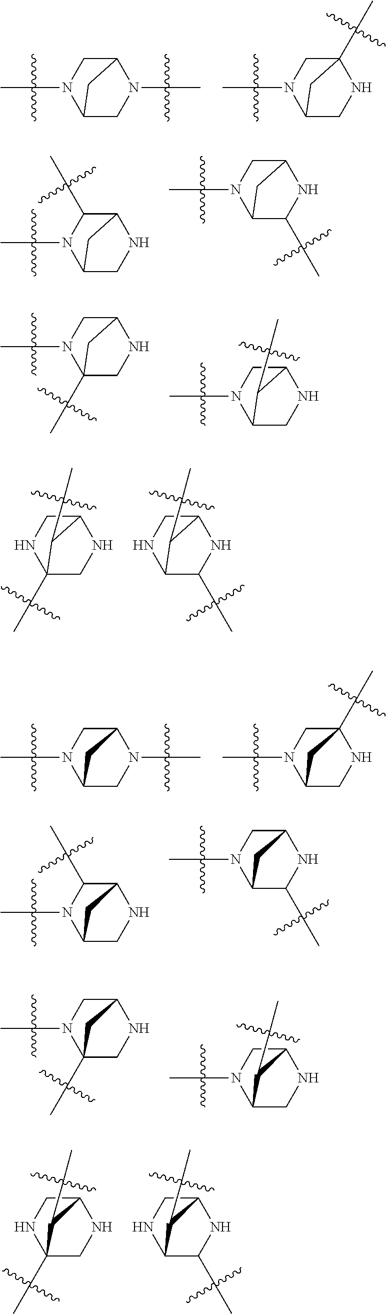 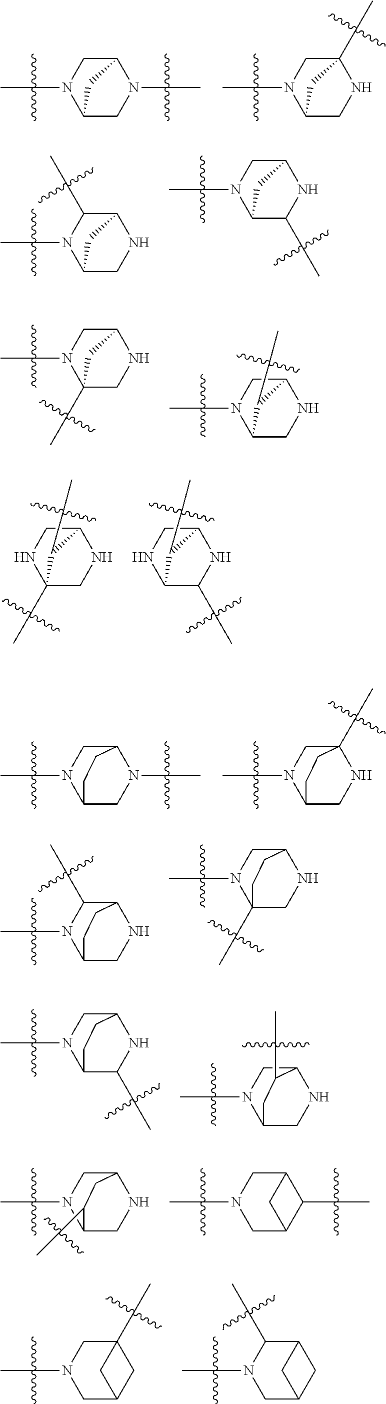 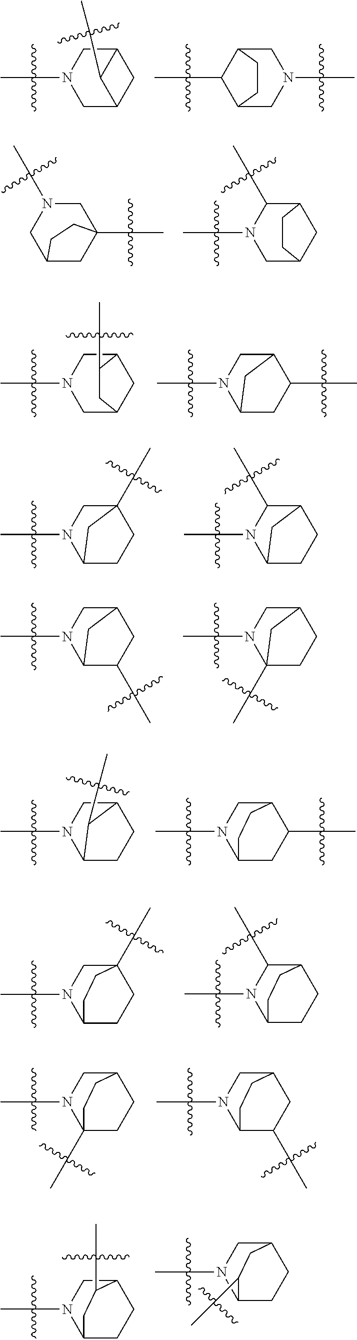 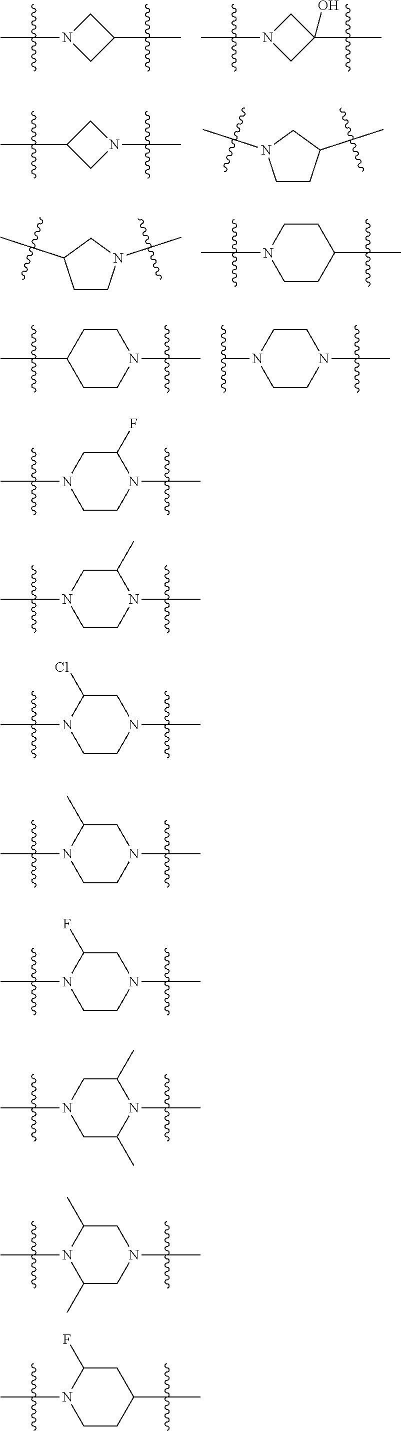 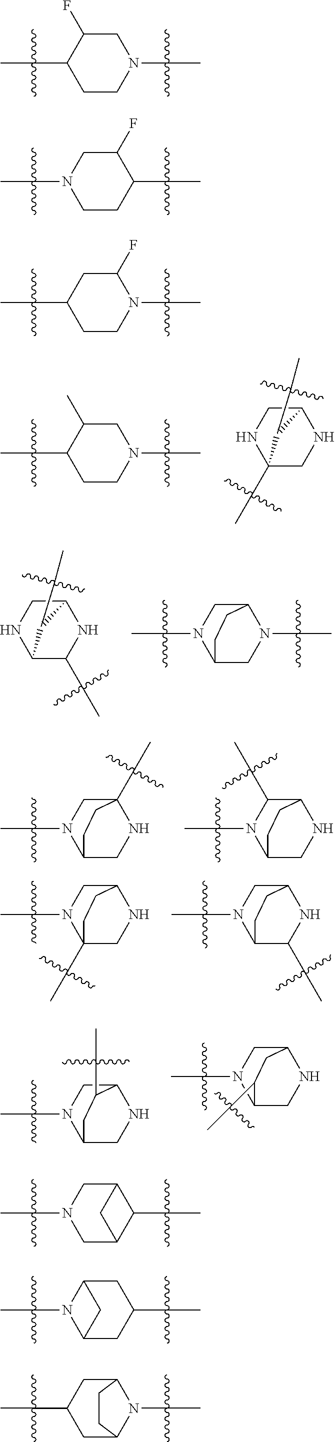 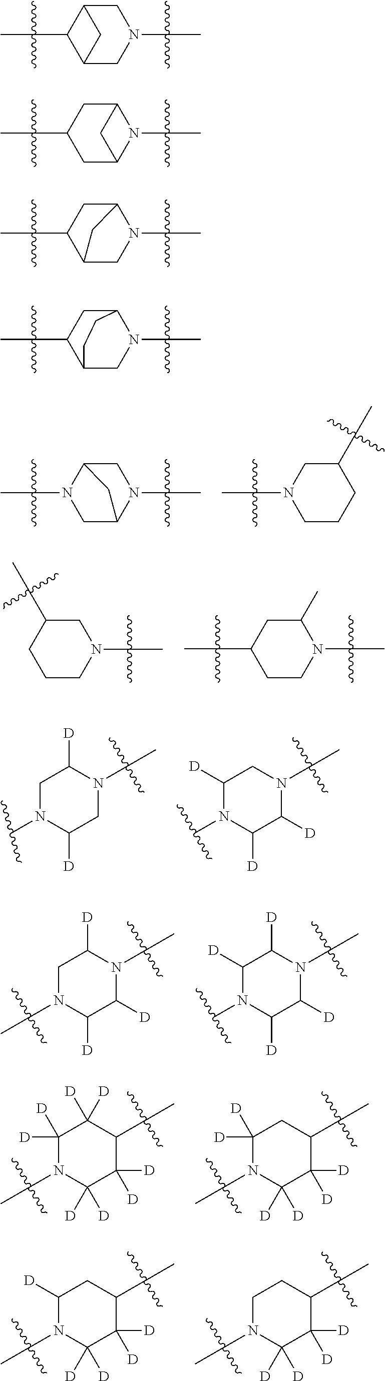 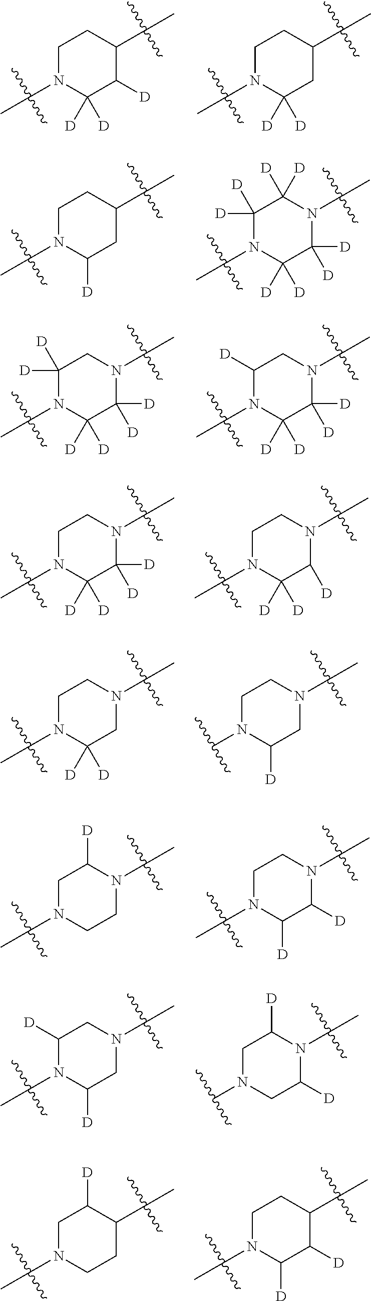 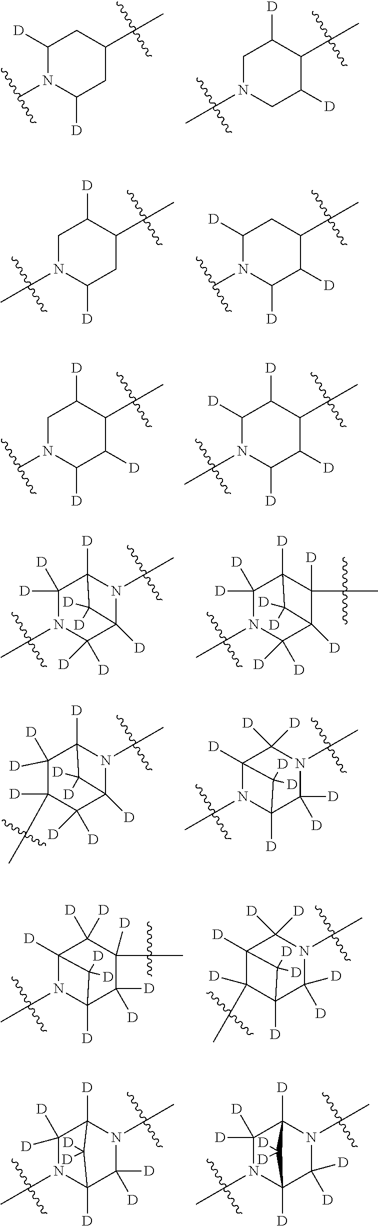 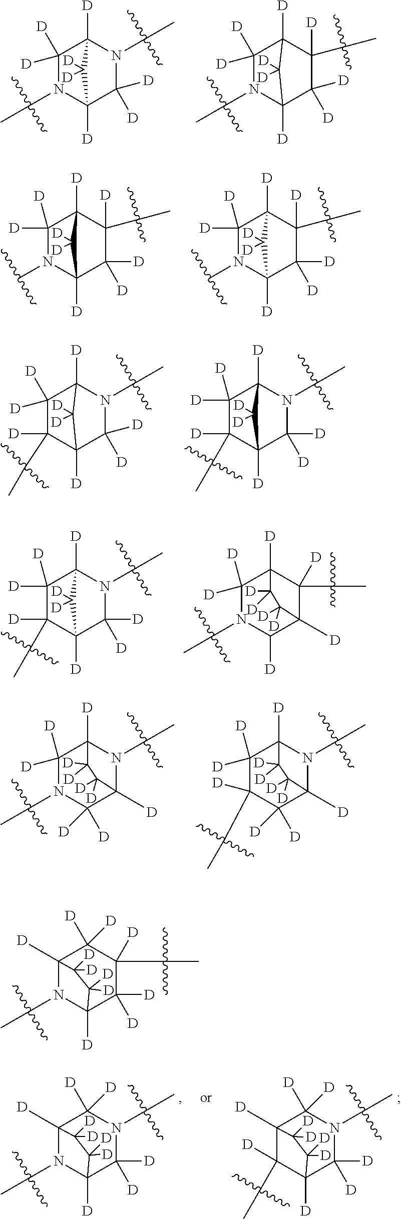
each ring A¹ is independently optionally substituted with a1 R^{y1} substituents, and each R^{y1} independently represents deuterium, C₁₋₆ alkyl, deuterated C₁₋₆ alkyl, halogenated C₁₋₆ alkyl, C₁₋₆ alkoxy, halogen, amino, hydroxy, mercapto, cyano, carboxyl, C₂₋₆ alkynyl, or C₂₋₆ alkenyl; and
a1 represents an integer of 0-20;
and/or
(ii) each ring A² independently represents 4-to 20-membered heterocyclylene, C₃₋₂₀cycloalkylene, C₅₋₂₀ arylene, or 5-to 20-membered heteroarylene; preferably each ring A² independently represents:
azetidinylene, oxetanylene, pyrrolidinylene, imidazolidinylene, pyrazolidylene, tetrahydrofuranylene, tetrahydropyranylene, tetrahydrothienylene, tetrahydrothiopyranylene, oxazolidinylene, thiazolidinylene, piperidinylene, piperazinylene, morpholinylene, thiomorpholinylene, azacycloheptanylene, azacyclooctylene, dioxacyclohexylene, azacycloheptanylene, azacyclooctylene, diazacycloheptanylene, diazacyclooctylene, 6-azabicyclo[3.1.1]heptanylene, 2,5-diazabicyclo[2.2.1]heptanylene, 3,6-diazabicyclo[3.1.1]heptanylene, 3-azabicyclo[3.2.1]octanylene, 3,8-diazabicyclo[3.2.1]octanylene, 3,8-diazabicyclo[3.2.1]octanylene, 2,5-diazabicyclo[2.2.2]octanylene, 5-to 20-membered azaspirocycloalkylene, cyclopropylene, cyclobutylene, cyclopentylene, cyclopentenylene, cyclohexylene, cyclohexenylene, cycloheptylene, cyclooctylene, decalinylene, octahydropentalenylene, octahydro-1H-indenylene, 2,3-dihydro-1H-indenylene, C₅-15 spiro-cycloalkylene, adamantanylene, noradamantanylene, norbornylene, phenylene, naphthylene, furanylene, oxazolylene, isoxazolylene, oxadiazolylene, thienylene, thiazolylene, isothiazolylene, thiadiazolylene, pyrrolylene, imidazolylene, pyrazolylene, triazolylene, pyridylene, pyrimidinylene, pyridazinylene, pyrazinylene, indolylene, isoindolylene, benzofuranylene, isobenzofuranylene, benzothienylene, indazolylene, benzimidazolylene, benzoxazolylene, benzisoxazolylene, benzothiazolylene, benzisothiazolylene, benzotriazolylene, benzo[2,1,3]oxadiazolylene, benzo[2,1,3]thiadiazolylene, benzo[1,2,3]thiadiazolylene, quinolinylene, isoquinolinylene, naphthyridinylene, cinnolinylene, quinazolinylene, quinoxalinylene, phthalazinylene, pyrazolo[1,5-a]pyridylene, pyrazolo[1,5-a]pyrimidinylene, imidazo[1,2-a]pyridylene, 1H-pyrrolo[3,2-b]pyridylene, 1H-pyrrolo[2,3-b]pyridylene, pyrrolo[2,1-b]thiazolylene, imidazo[2,1-b]thiazolylene, or 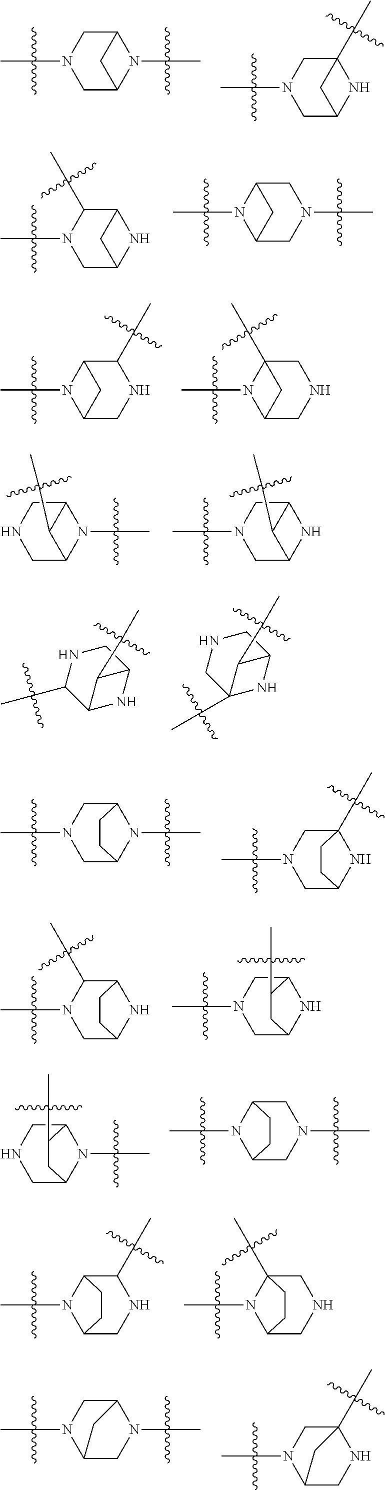 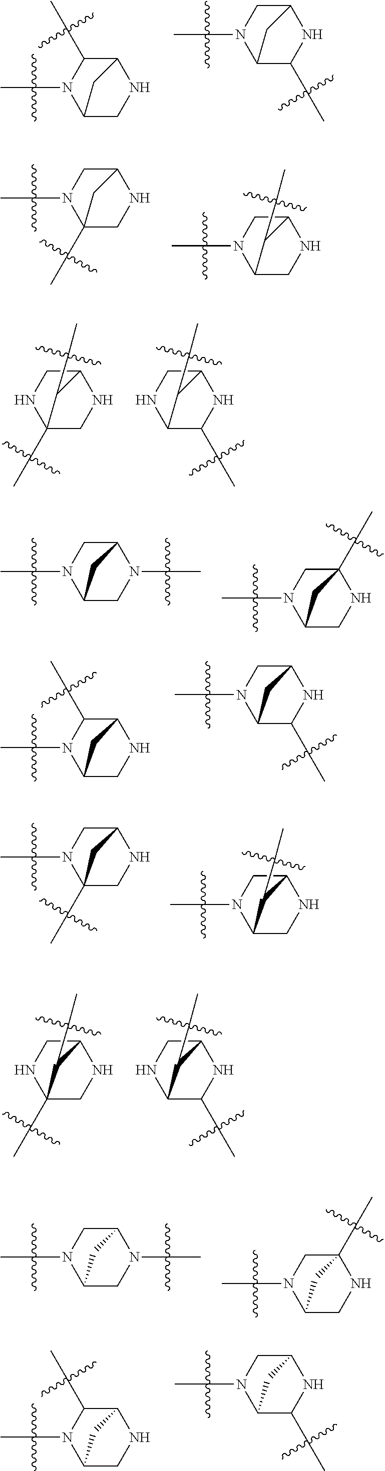 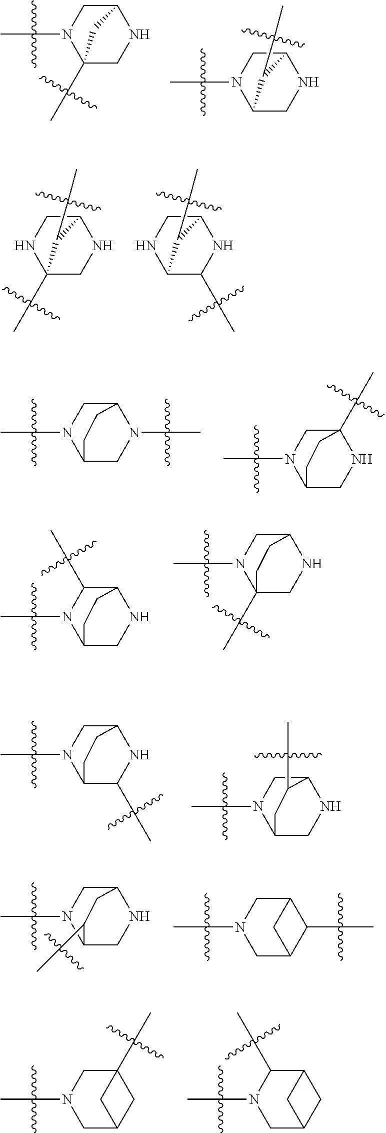 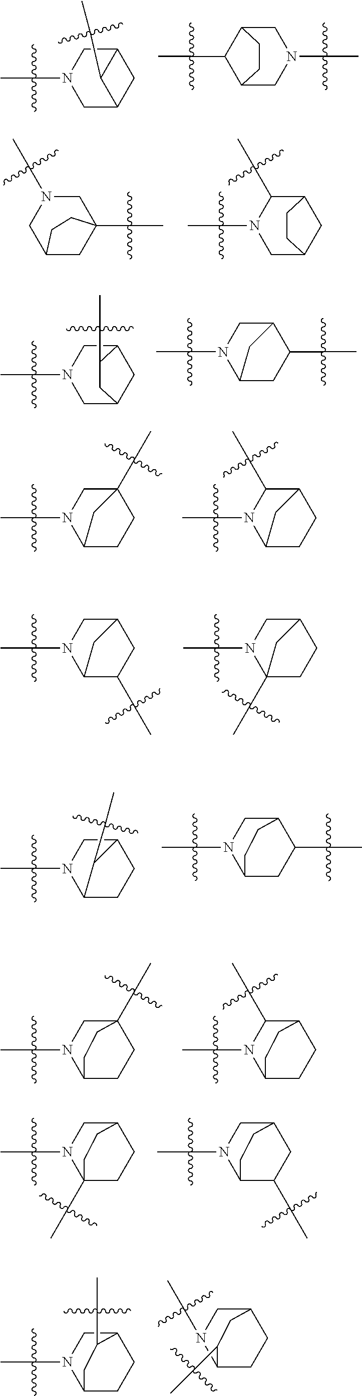 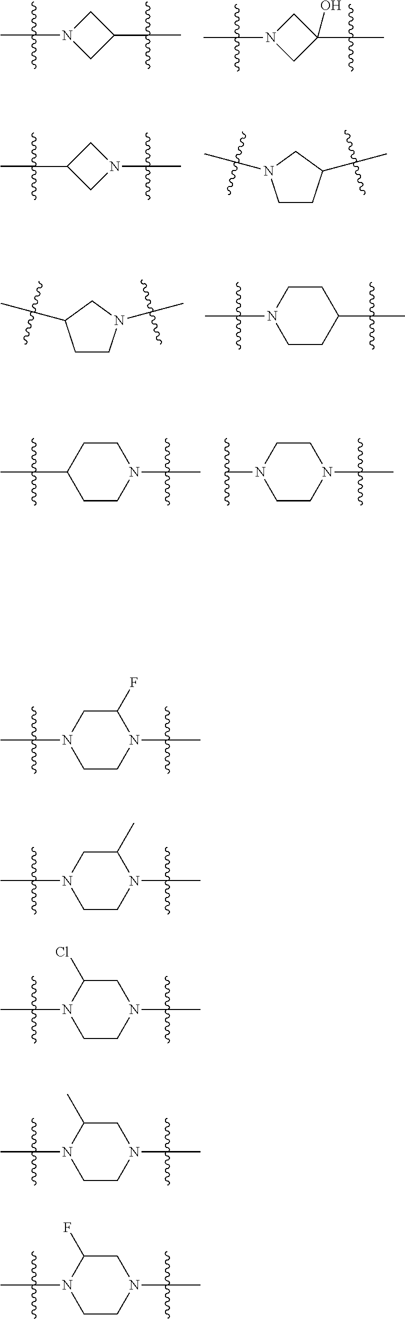 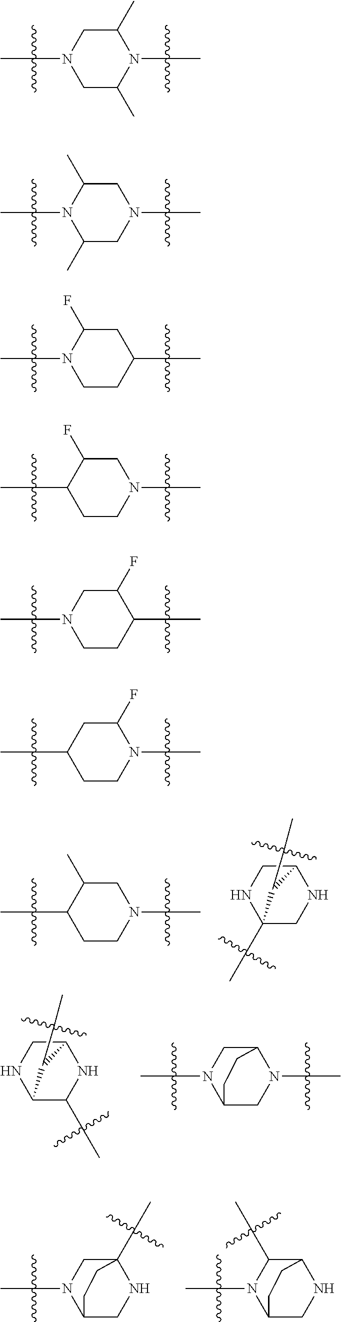 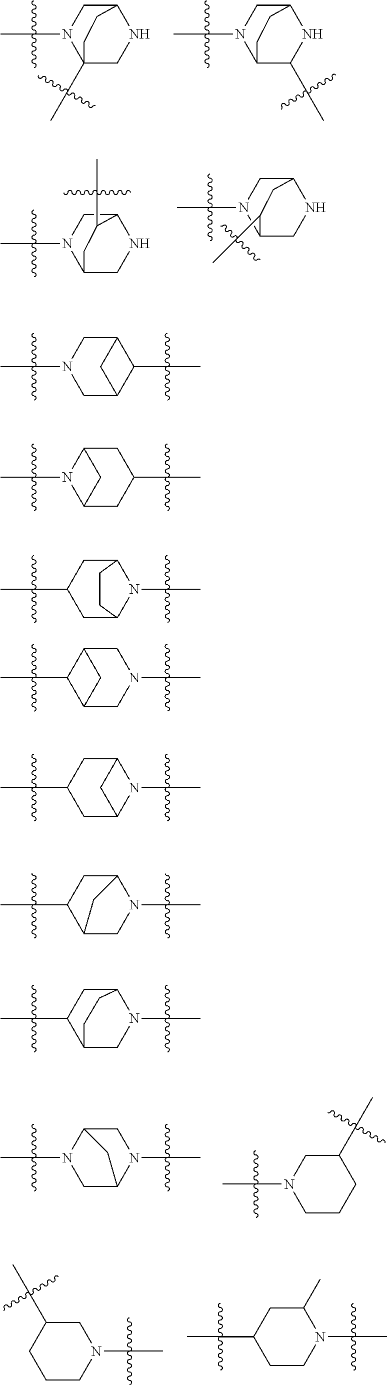 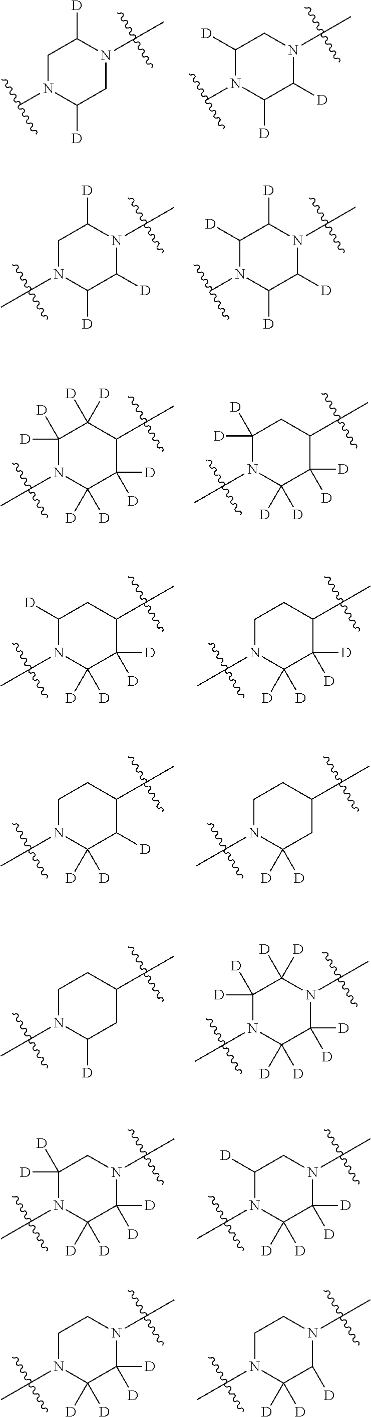 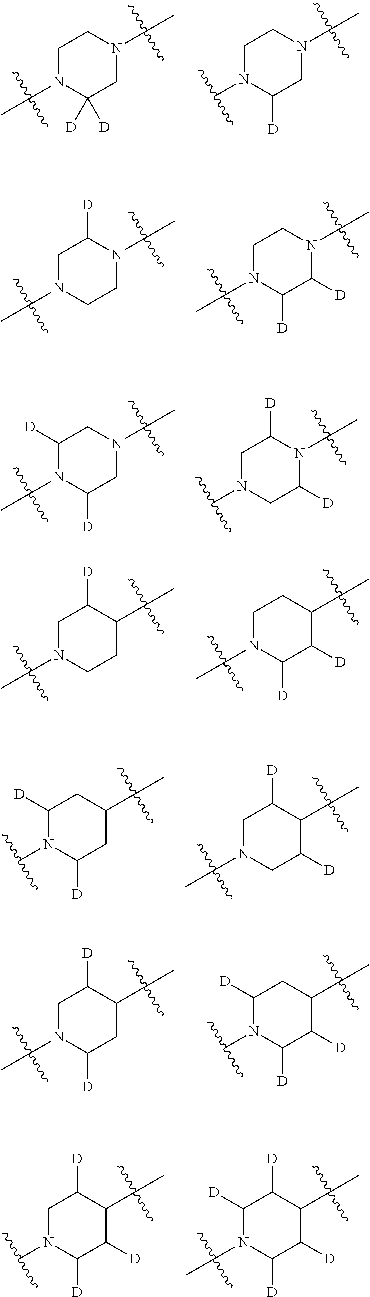 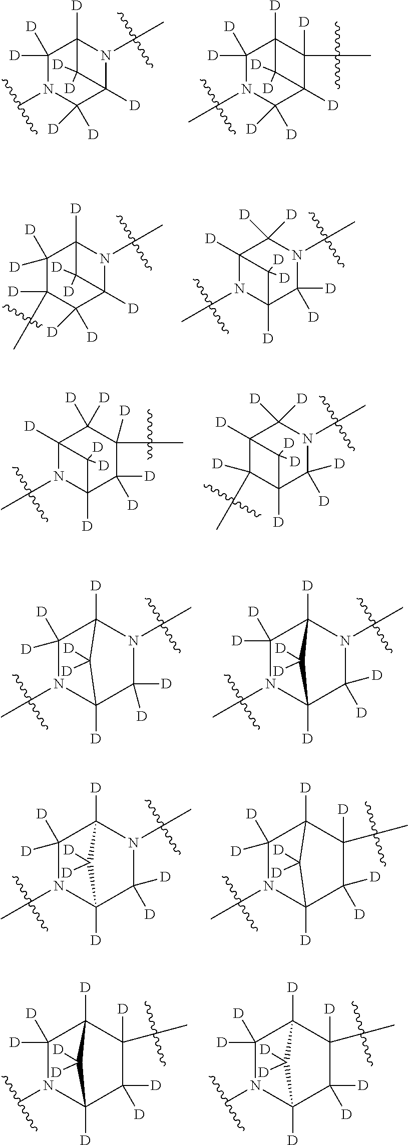 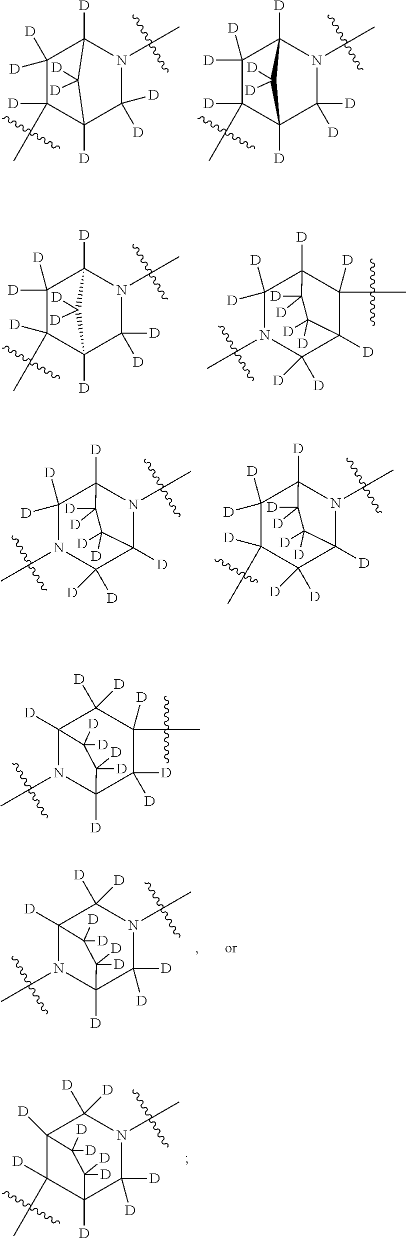
each ring A² is independently optionally substituted with a2 R^{y2} substituents, and each R^{y2} independently represents deuterium, C₁₋₆ alkyl, deuterated C₁₋₆ alkyl, halogenated C₁₋₆ alkyl, C₁₋₆ alkoxy, halogen, amino, hydroxy, mercapto, cyano, carboxyl, C₂₋₆ alkynyl, or C₂₋₆ alkenyl, and
a2 represents an integer of 0-20.

24. The compound of Formula (II) or salts, enantiomers, stereoisomers, solvates, isotopically enriched analogs, prodrugs, or polymorphs thereof as claimed in claim 21, wherein R_{w} represents: 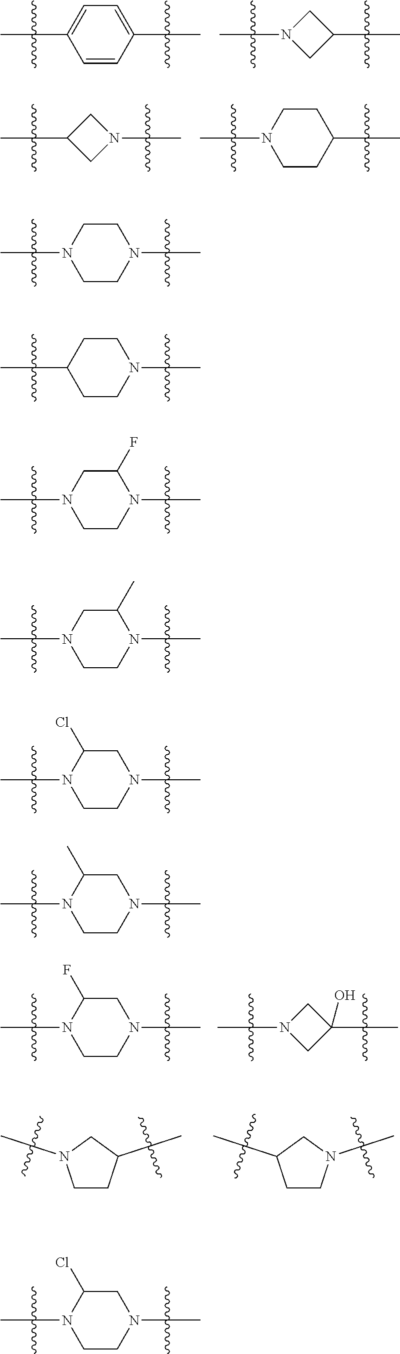 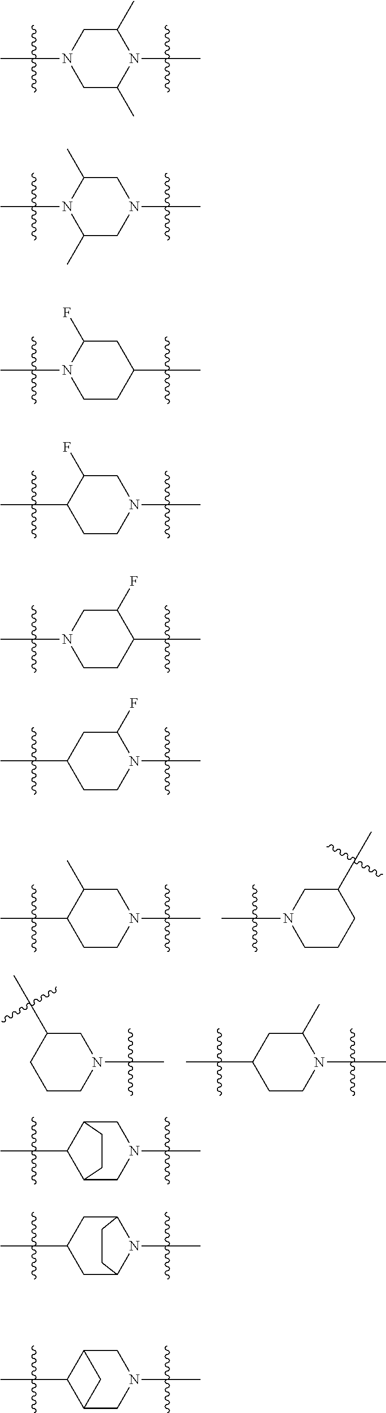 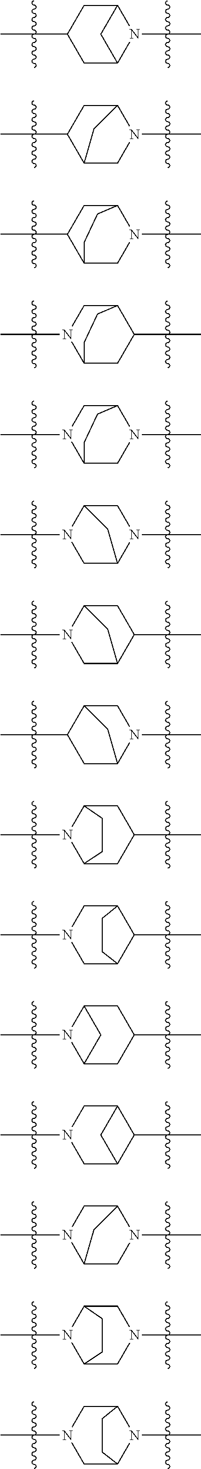 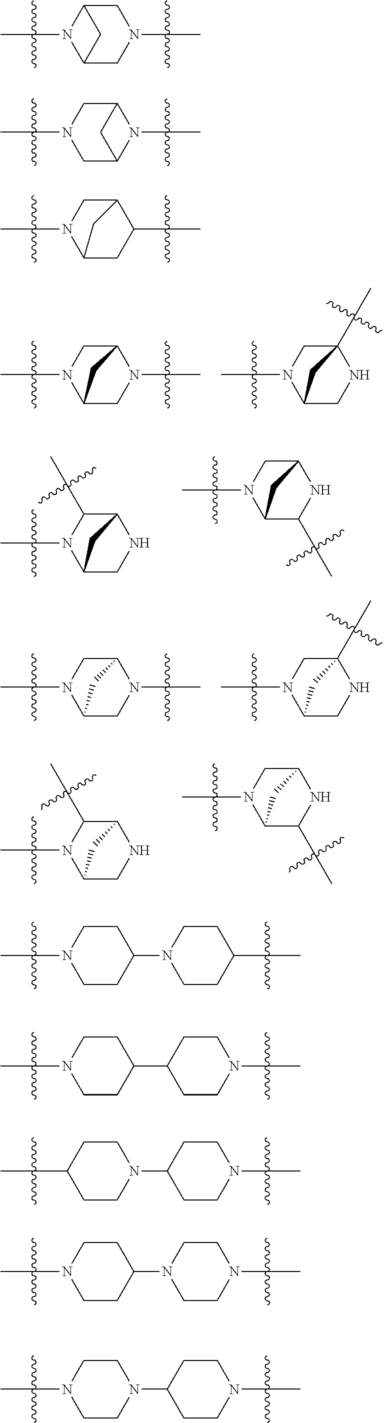 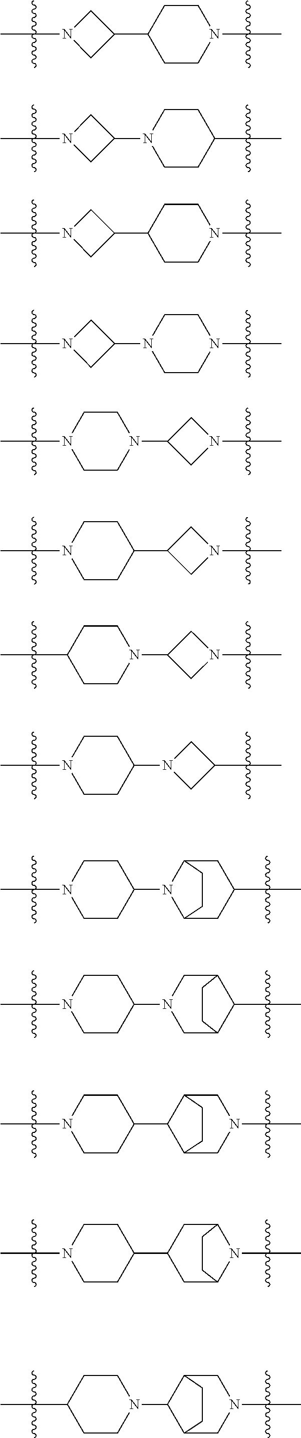 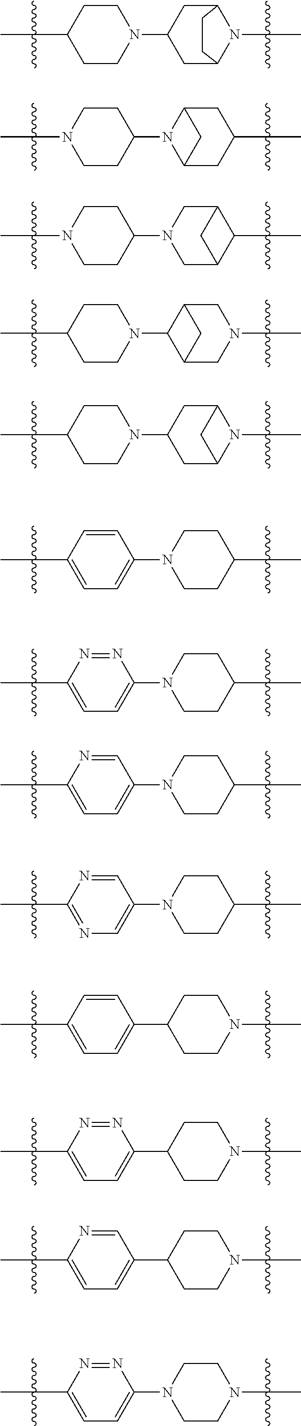 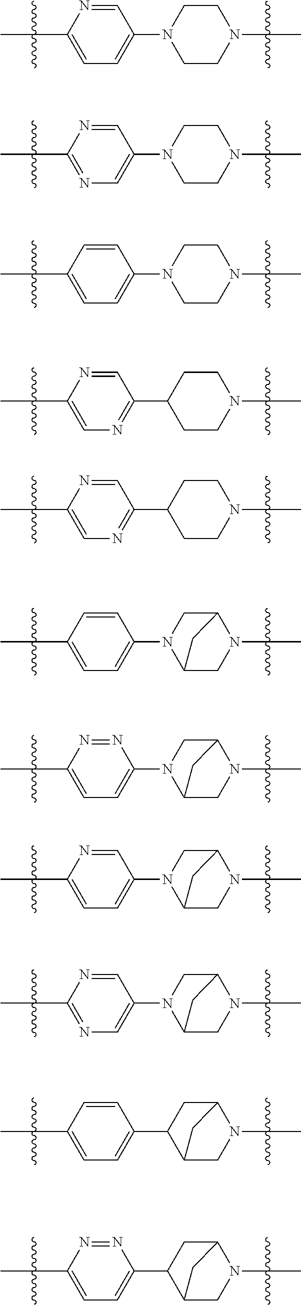 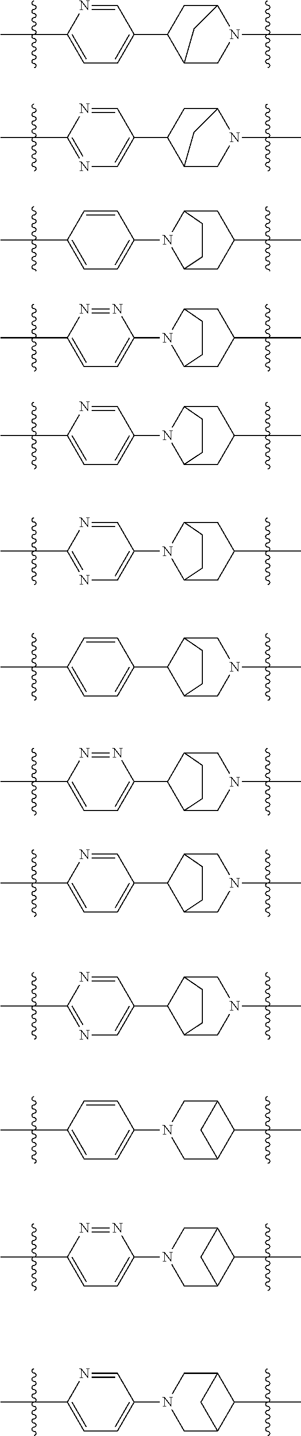 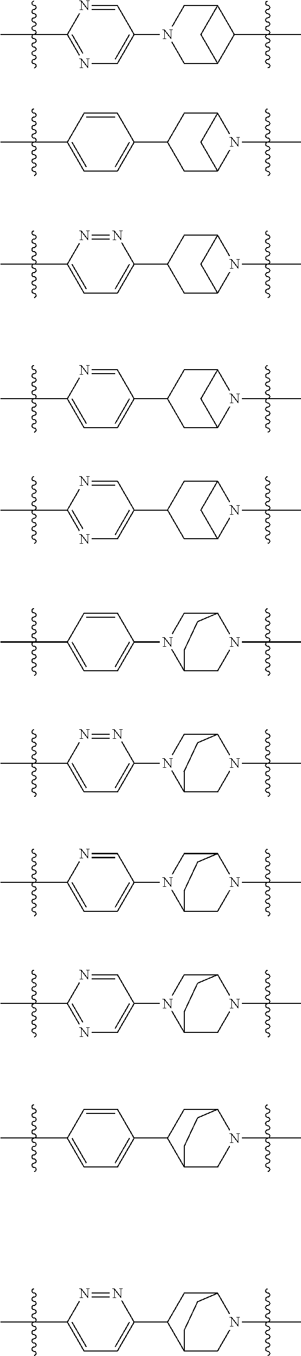 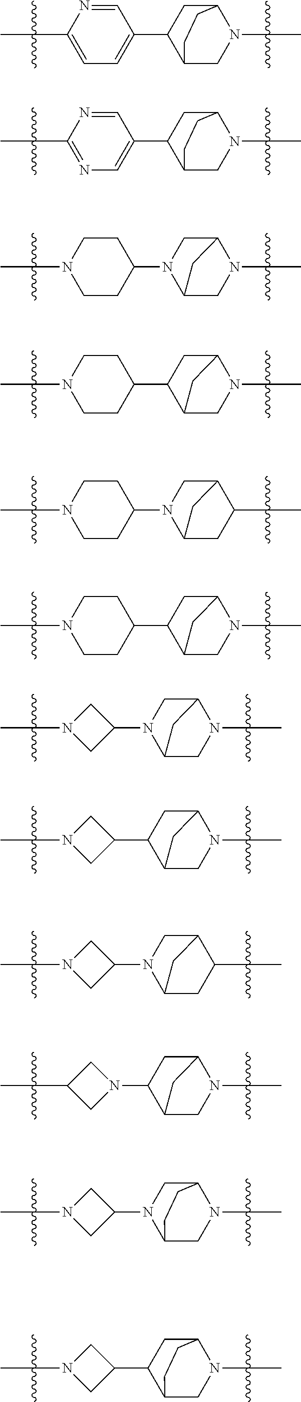 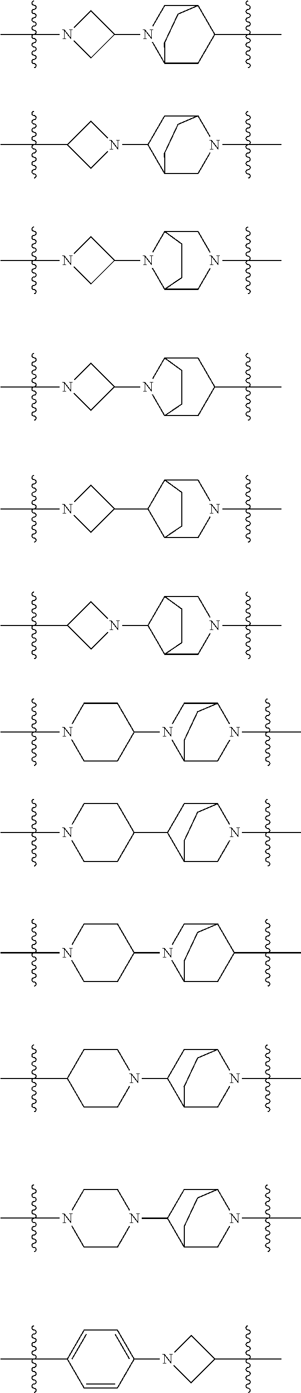 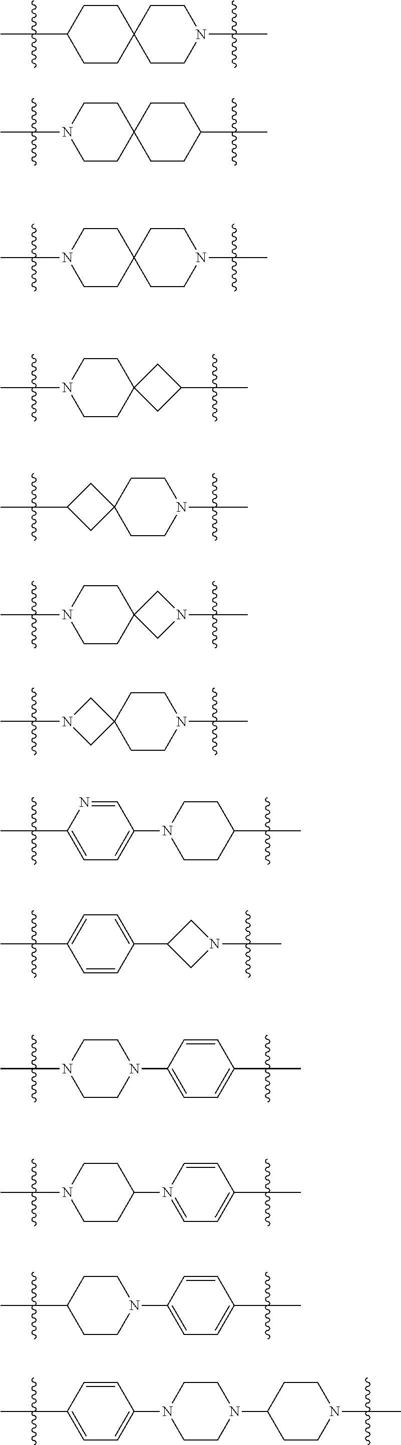 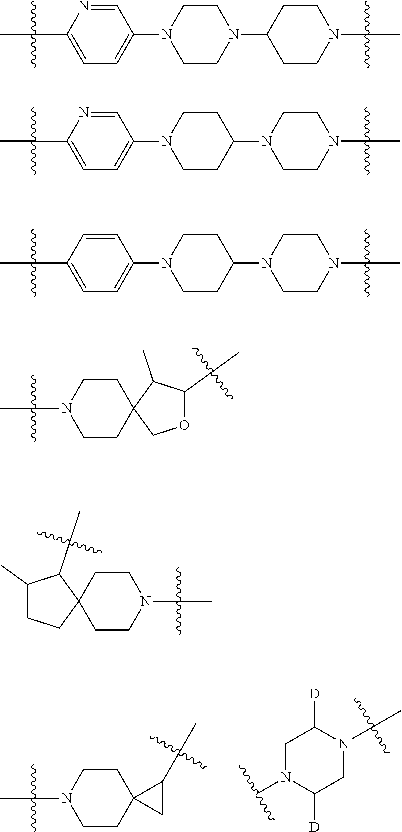 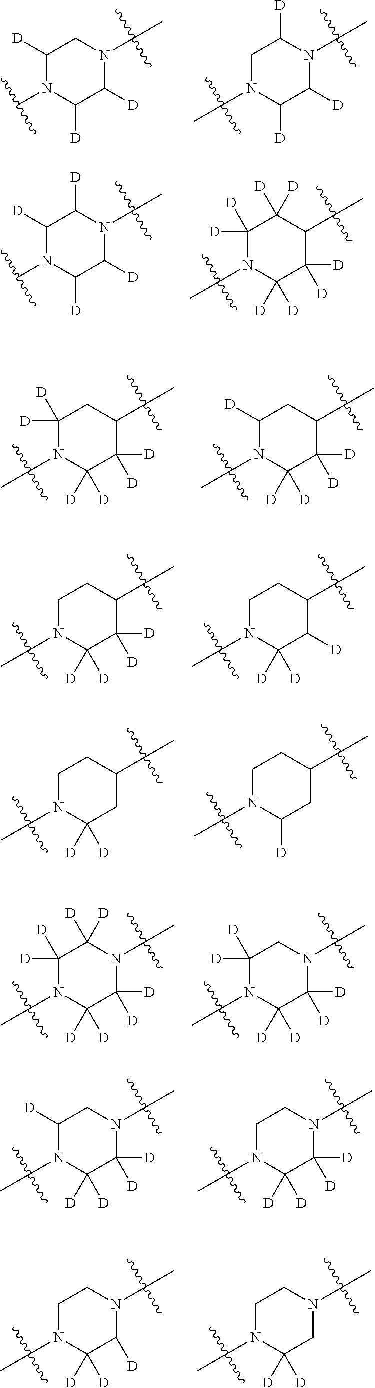 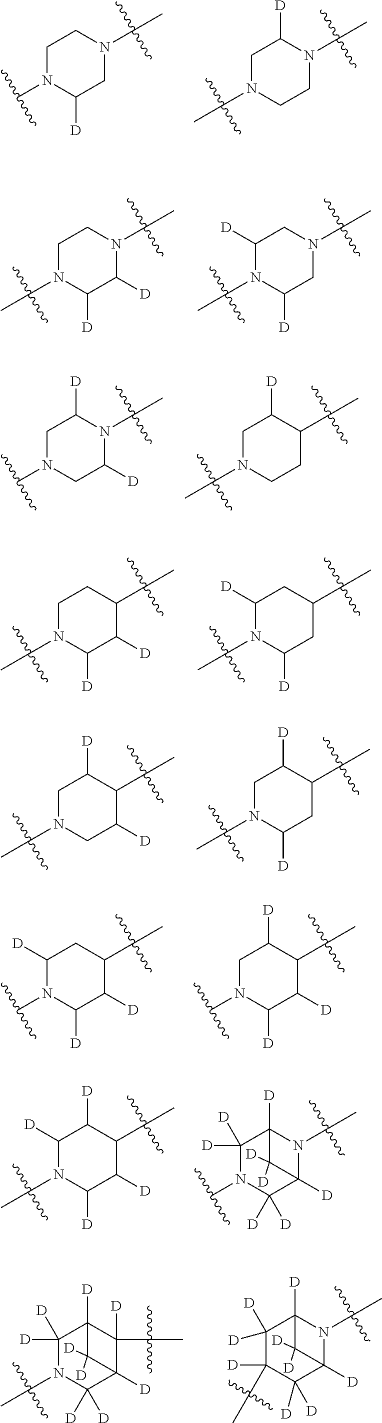 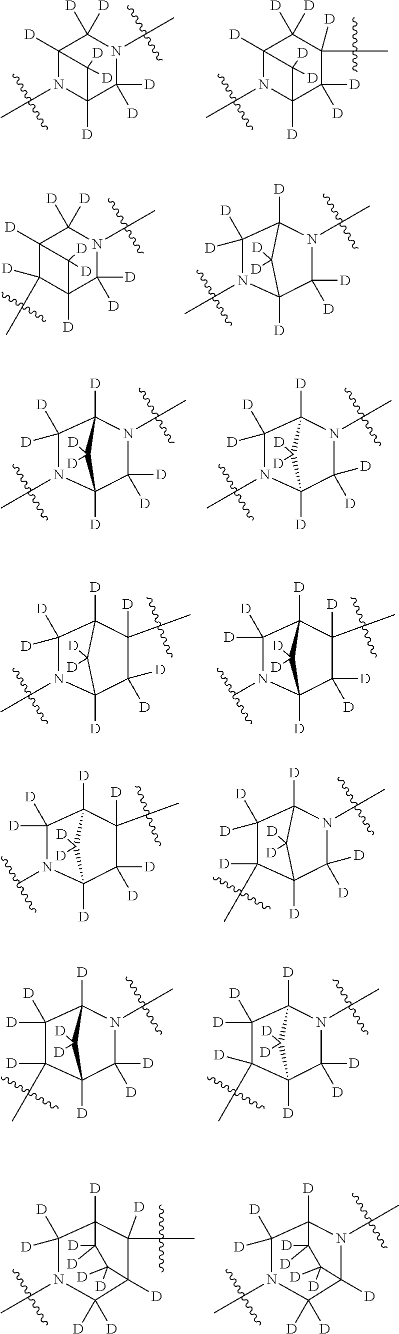 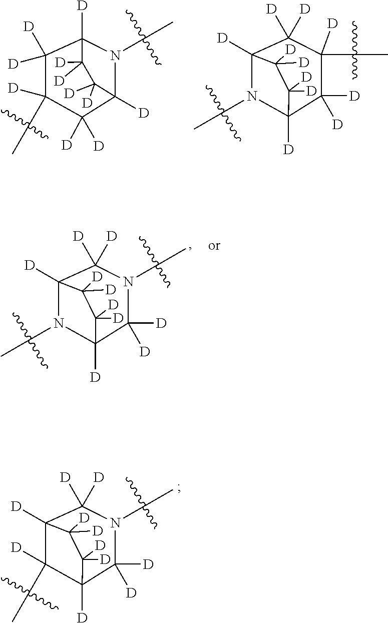 [SMILES: *N1C2([2H])C([2H])([2H])C(*)([2H])C([2H])([2H])C1([2H])C([2H])([2H])C2([2H])[2H] ; *N1C2([2H])C([2H])([2H])C(*)([2H])C([2H])([2H])C1([2H])C([2H])([2H])C2([2H])[2H] ; *N1C([2H])([2H])C2([2H])N(*)C([2H])(C1([2H])[2H])C([2H])([2H])C2([2H])[2H] ; "," ; "or" ; *N1C([2H])([2H])C2([2H])C(*)([2H])C([2H])(C1([2H])[2H])C([2H])([2H])C2([2H])[2H]]
wherein the groups are optionally substituted with a substituent selected from the group consisting of deuterium, C₁₋₆ alkyl, deuterated C₁₋₆ alkyl, halogenated C₁₋₆ alkyl, C₁₋₆ alkoxy, halogen, amino, hydroxy, mercapto, cyano, carboxyl, C₂₋₆ alkynyl, or C₂₋₆ alkenyl.

25. The compound of Formula (II) or salts, enantiomers, stereoisomers, solvates, isotopically enriched analogs, prodrugs, or polymorphs thereof as claimed in claim 21, wherein the R_{w1} represents a bond or optionally substituted linear or branched C₁₋₂₀ alkylene, wherein any one or more methylene groups in the main carbon chain skeleton of the linear or branched C₁₋₂₀ alkylene are optionally replaced by one or more groups selected from: Rₐ, R_{b}, and any combination thereof, wherein each R^{a} is independently selected from the group consisting of O, C(O), OC(O), S, S(O), S(O)₂, S(O)₂N(Rₐ₇), N(Rₐ₇)S(O)₂, C(O)N(Rₐ₇), N(Rₐ₇)C(O), N(Rₐ₇), and N(Rₐ₇)C(O)N(Rₐ₇), where each Raz is independently H or C₁₋₃ alkyl, and when any two or more methylene in the main carbon chain skeleton of the linear or branched C₁₋₂₀ alkylene are replaced by two or more Rₐ groups, the Rₐ groups are not directly connected to each other; and wherein each R^{b} is independently selected from the group consisting of: optionally substituted cycloalkylene (e.g., optionally substituted C₃₋₂₀cycloalkylene), optionally substituted arylene (e.g., optionally substituted C₅₋₂₀ arylene), optionally substituted heterocyclylene (e.g., optionally substituted 4-to 20-membered heterocyclylene), optionally substituted heteroarylene (e.g., optionally substituted 5-to 20-membered heteroarylene), alkynylene (e.g., C₂₋₆alkynylene), alkenylene (e.g., C₂₋₆alkenylene), and any combination thereof, wherein the cycloalkylene, arylene, heterocyclylene, and heteroarylene are each independently optionally substituted with a substituent selected from the group consisting of deuterium, hydroxy, amino, mercapto, nitro, halogen, cyano, oxo, C₁₋₆ alkyl, halogenated C₁₋₆ alkyl, deuterated C₁₋₆ alkyl, C₁₋₆ alkoxy, halogenated C₁₋₆ alkoxy, C₃₋₆ cycloalkyl, halogenated C₃₋₆ cycloalkyl, deuterated C₃₋₆ cycloalkyl, 4-to 10-membered heterocyclyl, and any combination thereof, and wherein one or more hydrogens of said linear or branched C₁₋₂₀ alkylene are optionally replaced by a substituent selected from the group consisting of deuterium, hydroxy, amino, mercapto, nitro, halogen, cyano, oxo, halogenated C₁₋₆ alkyl, deuterated C₁₋₆ alkyl, C₁₋₆ alkoxy, halogenated C₁₋₆ alkoxy, C₃₋₆ cycloalkyl, halogenated C₃₋₆ cycloalkyl, deuterated C₃₋₆ cycloalkyl, 4-to 10-membered heterocyclyl, and any combination thereof.

26. The compound of Formula (II) or salts, enantiomers, stereoisomers, solvates, isotopically enriched analogs, prodrugs, or polymorphs thereof as claimed in claim 21, wherein R_{w1} represents a bond, or R_{w1} represents:
—C₁₋₁₅ alkylene-;
—(C(Rₐ₅)(Rₐ₉))ₙ₁—(Rₐ(C(Rₐ₁₀)(Rₐ₁₁))ₙ₂)ₘ₃—;
—(C(Rₐ₅)(Rₐ₉))ₙ₁—(Rₐ(C(Rₐ₁₀)(Rₐ₁₁))ₙ₂)ₘ₃—(Rₐ(C(Rₐ₁₂)(Rₐ₁₃))ₙ₃)ₘ₄—;
—(C(Rₐ₅)(Rₐ₉))ₙ₁—(Rₐ(C(Rₐ₁₀)(Rₐ₁₁))ₙ₂)ₘ₃—(Rₐ(C(Rₐ₁₂)(Rₐ₁₃))ₙ₃)ₘ₄—(Rₐ(C(Rₐ₁₄)(Rₐ₁₅))ₙ₄)ₘ₅—;
—(C(Rₐ₅)(Rₐ₉))ₙ₁—((C(Rₐ₁₀)(Rₐ₁₁))ₙ₂Rₐ)ₘ₃—;
—(C(Rₐ₅)(Rₐ₉))ₙ₁—((C(Rₐ₁₀)(Rₐ₁₁))ₙ₂Rₐ)ₘ₃—((C(Rₐ₁₂)(Rₐ₁₃))ₙ₃Rₐ)ₘ₄—;
—(C(Rₐ₅)(Rₐ₉))ₙ₁—((C(Rₐ₁₀)(Rₐ₁₁))ₙ₂Rₐ)ₘ₃—((C(Rₐ₁₂)(Rₐ₁₃))ₙ₃Rₐ)ₘ₄—((C(Rₐ₁₄)(Rₐ₁₅))ₙ₄Rₐ)ₘ₅—;
—(C(Rₐ₅)(Rₐ₉))ₙ₁—(R_{b}(C(Rₐ₁₀)(Rₐ₁₁))ₙ₂)ₘ₃—;
—(C(Rₐ₈)(Rₐ₉))ₙ₁—(R_{b}(C(Rₐ₁₀)(Rₐ₁₁))ₙ₂)ₘ₃—(R_{b}(C(Rₐ₁₂)(Rₐ₁₃))ₙ₃)ₘ₄—;
—(C(Rₐ₅)(Rₐ₉))ₙ₁—(R_{b}(C(Rₐ₁₀)(Rₐ₁₁))ₙ₂)ₘ₃—(R_{b}(C(Rₐ₁₂)(Rₐ₁₃))ₙ₃)ₘ₄—(R_{b}(C(Rₐ₁₄)(Rₐ₁₅))ₙ₄)ₘ₅—;
—(C(Rₐ₈)(Rₐ₉))ₙ₁—((C(Rₐ₁₀)(Rₐ₁₁))ₙ₂R_{b})ₘ₃—;
—(C(Rₐ₅)(Rₐ₉))ₙ₁—((C(Rₐ₁₀)(Rₐ₁₁))ₙ₂R_{b})ₘ₃—((C(Rₐ₁₂)(Rₐ₁₃))ₙ₃R_{b})ₘ₄—;
—(C(Rₐ₅)(Rₐ₉))ₙ₁—((C(Rₐ₁₀)(Rₐ₁₁))ₙ₂R_{b})ₘ₃—((C(Rₐ₁₂)(Rₐ₁₃))ₙ₃R_{b})ₘ₄—((C(Rₐ₁₄)(Rₐ₁₅))ₙ₄R_{b})ₘ₅—;
—(C(Rₐ₅)(Rₐ₉))ₙ₁—(Rₐ—R_{b}—(C(Rₐ₁₀)(Rₐ₁₁))ₙ₂)ₘ₃—;
—(C(Rₐ₅)(Rₐ₉))ₙ₁—(Rₐ(C(Rₐ₁₀)(Rₐ₁₁))ₙ₂)ₘ₃—(R_{b}(C(Rₐ₁₂)(Rₐ₁₃))ₙ₃)ₘ₄—;
—(C(Rₐ₅)(Rₐ₉))ₙ₁—(R_{b}—Rₐ—(C(Rₐ₁₀)(Rₐ₁₁))ₙ₂)ₘ₃—;
—(C(Rₐ₅)(Rₐ₉))ₙ₁—(R_{b}(C(Rₐ₁₀)(Rₐ₁₁))ₙ₂)ₘ₃—(Rₐ(C(Rₐ₁₂)(Rₐ₁₃))ₙ₃)ₘ₄—;
—(C(Rₐ₅)(Rₐ₉))ₙ₁—((C(Rₐ₁₀)(Rₐ₁₁))ₙ₂—Rₐ—R_{b})ₘ₃—;
—(C(Rₐ₅)(Rₐ₉))ₙ₁—((C(Rₐ₁₀)(Rₐ₁₁))ₙ₂Rₐ)ₘ₃—((C(Rₐ₁₂)(Rₐ₁₃))ₙ₃R_{b})ₘ₄—;
—(C(Rₐ₅)(Rₐ₉))ₙ₁—((C(Rₐ₁₀)(Rₐ₁₁))ₙ₂—R_{b}—Rₐ)ₘ₃—, or
(C(Rₐ₅)(Rₐ₉))ₙ₁—((C(Rₐ₁₀)(Rₐ₁₁))ₙ₂R_{b})ₘ₃—((C(Rₐ₁₂)(Rₐ₁₃))ₙ₃Rₐ)ₘ₄—;
wherein each R^{a} is independently selected from the group consisting of O, C(O), OC(O), S, S(O), S(O)₂, S(O)₂N(Rₐ₇), N(Rₐ₇)S(O)₂, C(O)N(Rₐ₇), N(Rₐ₇)C(O), N(Rₐ₇), and N(Rₐ₇)C(O)N(Rₐ₇), wherein each Rₐ₇ independently represents H or C₁₋₃ alkyl; and each R_{b} is independently selected from the group consisting of optionally substituted cycloalkylene (e.g., optionally substituted C₃₋₂₀cycloalkylene), optionally substituted arylene (e.g., optionally substituted C₅₋₂₀ arylene), optionally substituted heterocyclylene (e.g., optionally substituted 4-to 20-membered heterocyclylene), optionally substituted heteroarylene (e.g., 5-to 20-membered heteroarylene), alkynylene (e.g., C₂₋₆alkynylene), alkenylene (e.g., C₂₋₆alkenylene), and any combination thereof, wherein the cycloalkylene, arylene, heterocyclylene, and heteroarylene are each independently optionally substituted with a substituent selected from the group consisting of deuterium, hydroxy, amino, mercapto, nitro, halogen, cyano, oxo, C₁₋₆ alkyl, halogenated C₁₋₆ alkyl, deuterated C₁₋₆ alkyl, C₁₋₆ alkoxy, halogenated C₁₋₆ alkoxy, C₃₋₆ cycloalkyl, halogenated C₃₋₆ cycloalkyl, deuterated C₃₋₆ cycloalkyl, 4-to 10-membered heterocyclyl, and any combination thereof;
Rₐ₅, Rₐ₉, Rₐ₁₀, Rₐ₁₁, Rₐ₁₂, Rₐ₁₃, Rₐ₁₄, and Rₐ₁₅ each independently represent H, deuterium, hydroxy, amino, mercapto, nitro, halogen, cyano, oxo, halogenated C₁₋₆ alkyl, deuterated C₁₋₆ alkyl, C₁₋₆ alkoxy, halogenated C₁₋₆ alkoxy, C₃₋₆ cycloalkyl, halogenated C₃₋₆ cycloalkyl, deuterated C₃₋₆ cycloalkyl, 4-to 10-membered heterocyclyl, and any combination thereof, and
n1, n2, n3, n4, m3, m4, and m5 each independently represent an integer of 1, 2, 3, 4, 5, 6, 7, 8, 9, or 10; and
any one or more hydrogens of said C₁₋₁₅ alkylene are optionally replaced by a substituent selected from: deuterium, hydroxy, amino, mercapto, nitro, halogen, cyano, oxo, halogenated C₁₋₆ alkyl, deuterated C₁₋₆ alkyl, C₁₋₆ alkoxy, halogenated C₁₋₆ alkoxy, C₃₋₆ cycloalkyl, halogenated C₃₋₆ cycloalkyl, deuterated C₃₋₆ cycloalkyl, 4-to 10-membered heterocyclyl, and any combination thereof.

27. The compound of Formula (I) or salts, enantiomers, stereoisomers, solvates, isotopically enriched analogs, prodrugs, or polymorphs thereof as claimed in claim 21, wherein the R_{w1} represents a structure of the following Formulae:
—(C(Rₐ₅)(Rₐ₉))ₙ₁—(O(C(Rₐ₁₀)(Rₐ₁₁))ₙ₂)ₘ₃—;
—(C(Rₐ₈)(Rₐ₉))ₙ₁—(O(C(Rₐ₁₀)(Rₐ₁₁))ₙ₂)ₘ₃—(O(C(Rₐ₁₂)(Rₐ₁₃))ₙ₃)ₘ₄—;
—(C(Rₐ₅)(Rₐ₉))ₙ₁—(O(C(Rₐ₁₀)(Rₐ₁₁))ₙ₂)ₘ₃—(O(C(Rₐ₁₂)(Rₐ₁₃))ₙ₃)ₘ₄—(O(C(Rₐ₁₄)(Rₐ₁₅))ₙ₄)ₘ₅—;
—(C(Rₐ₅)(Rₐ₉))ₙ₁—((C(Rₐ₁₀)(Rₐ₁₁))ₙ₂₀)ₘ₃—;
—(C(Rₐ₈)(Rₐ₉))ₙ₁—((C(Rₐ₁₀)(Rₐ₁₁))ₙ₂₀)ₘ₃—((C(Rₐ₁₂)(Rₐ₁₃))ₙ₃₀)ₘ₄—;
—(C(Rₐ₅)(Rₐ₉))ₙ₁—((C(Rₐ₁₀)(Rₐ₁₁))ₙ₂₀)ₘ₃—((C(Rₐ₁₂)(Rₐ₁₃))ₙ₃₀)ₘ₄—((C(Rₐ₁₄)(Rₐ₁₅))ₙ₄₀)ₘ₅—;
—(C(Rₐ₈)(Rₐ₉))ₙ₁—(N(Rₐ₇)(C(Rₐ₁₀)(Rₐ₁₁))ₙ₂)ₘ₃—;
—(C(Rₐ₅)(Rₐ₉))ₙ₁—(N(Rₐ₇)(C(Rₐ₁₀)(Rₐ₁₁))ₙ₂)ₘ₃—(N(Rₐ₇)(C(Rₐ₁₂)(Rₐ₁₃))ₙ₃)ₘ₄—;
—(C(Rₐ₅)(Rₐ₉))ₙ₁—(N(Rₐ₇)(C(Rₐ₁₀)(Rₐ₁₁))ₙ₂)ₘ₃—(N(Rₐ₇)(C(Rₐ₁₂)(Rₐ₁₃))ₙ₃)ₘ₄—(N(Rₐ₇)(C(Rₐ₁₄)(Rₐ₁₅))ₙ₄)ₘ₅—;
—(C(Rₐ₅)(Rₐ)ₙ₁—(C(Rₐ₁₀)(Rₐ₁₁)ₙ₂N(Rₐ₇)ₘ₃—;
—(C(Rₐ₅)(Rₐ₉))ₙ₁—((C(Rₐ₁₀)(Rₐ₁₁))ₙ₂N(Rₐ₇))ₘ₃—((C(Rₐ₁₂)(Rₐ₁₃))ₙ₃N(Rₐ₇))ₘ₄—;
—(C(Rₐ₅)(Rₐ₉))ₙ₁—((C(Rₐ₁₀)(Rₐ₁₁))ₙ₂N(Rₐ₇))ₘ₃—((C(Rₐ₁₂)(Rₐ₁₃))ₙ₃N(Rₐ₇))ₘ₄—((C(Rₐ₁₄)(Rₐ₁₅))ₙ₄N(Rₐ₇))ₘ₅—;
—(C(Rₐ₅)(Rₐ₉))ₙ₁—(C(O)N(Rₐ₇)—(C(Rₐ₁₀)(Rₐ₁₁))ₙ₂)ₘ₃—;
—(C(Rₐ₅)(Rₐ₉))ₙ₁—(C(O)N(Rₐ₇)—(C(Rₐ₁₀)(Rₐ₁₁))ₙ₂)ₘ₃—(C(O)N(Rₐ₇)—(C(Rₐ₁₂)(Rₐ₁₃))ₙ₃)ₘ₄—;
—(C(Rₐ₈)(Rₐ₉))ₙ₁—(C(O)N(Rₐ₇)—(C(Rₐ₁₀)(Rₐ₁₁))ₙ₂)ₘ₃—(C(O)N(Rₐ₇)—(C(Rₐ₁₂)(Rₐ₁₃))ₙ₃)ₘ₄—(C(O)N(Rₐ₇)—(C(Rₐ₁₄)(Rₐ₁₅))ₙ₄)ₘ₅—;
—(C(Rₐ₅)(Rₐ₉))ₙ₁—((C(Rₐ₁₀)(Rₐ₁₁))ₙ₂—C(O)N(Rₐ₇))ₘ₃—;
—(C(Rₐ₅)(Rₐ₉))ₙ₁—((C(Rₐ₁₀)(Rₐ₁₁))ₙ₂—C(O)N(Rₐ₇))ₘ₃—((C(Rₐ₁₂)(Rₐ₁₃))ₙ₃—C(O)N(Rₐ₇))ₘ₄—;
—(C(Rₐ₅)(Rₐ₉))ₙ₁—((C(Rₐ₁₀)(Rₐ₁₁))ₙ₂—C(O)N(Rₐ₇))ₘ₃—((C(Rₐ₁₂)(Rₐ₁₃))ₙ₃—C(O)N(Rₐ₇))ₘ₄—((C(Rₐ₁₄)(Rₐ₁₅))ₙ₄—C(O)N(Rₐ₇))ₘ₅—;
—(C(Rₐ₅)(Rₐ₉))ₙ₁—(C(O)N(Rₐ₇)—(C(Rₐ₁₀)(Rₐ₁₁))ₙ₂)ₘ₃—(O—(C(Rₐ₁₂)(Rₐ₁₃))ₙ₃)ₘ₄—;
—(C(Rₐ₅)(Rₐ₉))ₙ₁—C(O)N(Rₐ₇)—(C(Rₐ₁₀)(Rₐ₁₁))ₙ₂—(O(C(Rₐ₁₂)(Rₐ₁₃))ₙ₃)ₘ₃—;
—(C(Rₐ₈)(Rₐ₉))ₙ₁—((C(Rₐ₁₀)(Rₐ₁₁))ₙ₂—C(O)N(Rₐ₇))ₘ₃—((C(Rₐ₁₂)(Rₐ₁₃))ₙ₃₀)ₘ₄—;
—(C(Rₐ₅)(Rₐ₉))ₙ₁—(C(Rₐ₁₀)(Rₐ₁₁))ₙ₂—C(O)N(Rₐ₇)—((C(Rₐ₁₂)(Rₐ₁₃))ₙ₃₀)ₘ₃—;
—(C(Rₐ₅)(Rₐ₉))ₙ₁—(N(Rₐ₇)C(O)—(C(Rₐ₁₀)(Rₐ₁₁))ₙ₂)ₘ₃—;
—(C(Rₐ₅)(Rₐ₉))ₙ₁—(N(Rₐ₇)C(O)—(C(Rₐ₁₀)(Rₐ₁₁))ₙ₂)ₘ₃—(O—(C(Rₐ₁₂)(Rₐ₁₃)))ₙ₃)ₘ₄—;
—(C(Rₐ₅)(Rₐ₉))ₙ₁—(N(Rₐ₇)C(O)—(C(Rₐ₁₀)(Rₐ₁₁))ₙ₂)ₘ₃—(O—(C(Rₐ₁₂)(Rₐ₁₃))ₙ₃)ₘ₄—(O—(C(Rₐ₁₄)(Rₐ₁₅))ₙ₄)ₘ₅—;
—(C(Rₐ₅)(Rₐ₉))ₙ₁—N(Rₐ₇)C(O)—(C(Rₐ₁₀)(Rₐ₁₁))ₙ₂—(O(C(Rₐ₁₂)(Rₐ₁₃))ₙ₃)ₘ₃—;
—(C(Rₐ₈)(Rₐ₉))ₙ₁—N(Rₐ₇)C(O)N(Rₐ₇)—(C(Rₐ₁₀)(Rₐ₁₁))ₙ₂—;
—(C(Rₐ₈)(Rₐ₉))ₙ₁—((C(Rₐ₁₀)(Rₐ₁₁))ₙ₂—N(Rₐ₇)C(O))ₘ₃—;
—(C(Rₐ₅)(Rₐ₉))ₙ₁—((C(Rₐ₁₀)(Rₐ₁₁))ₙ₂—N(Rₐ₇)C(O))ₘ₃—((C(Rₐ₁₂)(Rₐ₁₃)))ₙ₃—O)ₘ₄—;
—(C(Rₐ₅)(Rₐ₉))ₙ₁—((C(Rₐ₁₀)(Rₐ₁₁))ₙ₂—N(Rₐ₇)C(O))ₘ₃—((C(Rₐ₁₂)(Rₐ₁₃))ₙ₃₀)ₘ₄—((C(Rₐ₁₄)(Rₐ₁₅))ₙ₄₀)ₘ₅—;
—(C(Rₐ₅)(Rₐ₉))ₙ₁—(C(Rₐ₁₀)(Rₐ₁₁))ₙ₂—N(Rₐ₇)C(O)—((C(Rₐ₁₂)(Rₐ₁₃))ₙ₃₀)ₘ₃—;
—(C(Rₐ₅)(Rₐ₉))ₙ₁-(arylene-(C(Rₐ₁₀)(Rₐ₁₁))ₙ₂)ₘ₃—;
—(C(Rₐ₅)(Rₐ₉))ₙ₁-(arylene-(C(Rₐ₁₀)(Rₐ₁₁))ₙ₂)ₘ₃-arylene-(C(Rₐ₁₂)(Rₐ₁₃))ₙ₃—;
—(C(Rₐ₅)(Rₐ₉))ₙ₁—((C(Rₐ₁₀)(Rₐ₁₁))ₙ₂-arylene)ₘ₃-;
—(C(Rₐ₅)(Rₐ₉))ₙ₁—((C(Rₐ₁₀)(Rₐ₁₁))ₙ₂-arylene)ₘ₃-(C(Rₐ₁₂)(Rₐ₁₃))ₙ₃-arylene-;
—(C(Rₐ₅)(Rₐ₉))ₙ₁-(heterocyclylene-(C(Rₐ₁₀)(Rₐ₁₁))ₙ₂)ₘ₃—;
—(C(Rₐ₅)(Rₐ₉))ₙ₁-(heterocyclylene-(C(Rₐ₁₀)(Rₐ₁₁))ₙ₂)ₘ₃-(heterocyclylene-(C(Rₐ₁₂)(Rₐ₁₃))ₙ₃)ₘ₄—;
—(C(Rₐ₅)(Rₐ₉))ₙ₁—((C(Rₐ₁₀)(Rₐ₁₁))ₙ₂-heterocyclylene)ₘ₃-;
—(C(Rₐ₅)(Rₐ₉))ₙ₁—((C(Rₐ₁₀)(Rₐ₁₁))ₙ₂-heterocyclylene)ₘ₃-((C(Rₐ₁₂)(Rₐ₁₃))ₙ₃-heterocyclylene)ₘ₄-;
—(C(Rₐ₅)(Rₐ₉))ₙ₁-(heteroarylene-(C(Rₐ₁₀)(Rₐ₁₁))ₙ₂)ₘ₃—;
—(C(Rₐ₅)(Rₐ₉))ₙ₁-(heteroarylene-(C(Rₐ₁₀)(Rₐ₁₁))ₙ₂)ₘ₃-(heteroarylene-(C(Rₐ₁₂)(Rₐ₁₃))ₙ₃)ₘ₄—;
—(C(Rₐ₅)(Rₐ₉))ₙ₁—((C(Rₐ₁₀)(Rₐ₁₁))ₙ₂-heteroarylene)ₘ₃-;
—(C(Rₐ₈)(Rₐ₉))ₙ₁—((C(Rₐ₁₀)(Rₐ₁₁))ₙ₂-heteroarylene)ₘ₃-((C(Rₐ₁₂)(Rₐ₁₃))ₙ₃-heteroarylene)ₘ₄-;
—(C(Rₐ₈)(Rₐ₉))ₙ₁-(cycloalkylene-(C(Rₐ₁₀)(Rₐ₁₁))ₙ₂)ₘ₃—;
—(C(Rₐ₅)(Rₐ₉))ₙ₁-(cycloalkylene-(C(Rₐ₁₀)(Rₐ₁₁))ₙ₂)ₘ₃-(cycloalkylene-(C(Rₐ₁₂)(Rₐ₁₃))ₙ₃)ₘ₄—;
—(C(Rₐ₅)(Rₐ₉))ₙ₁—((C(Rₐ₁₀)(Rₐ₁₁))ₙ₂-cycloalkylene)ₘ₃-, or
(C(Rₐ₅)(Rₐ₉))ₙ₁—((C(Rₐ₁₀)(Rₐ₁₁))ₙ₂-cycloalkylene)ₘ₃-((C(Rₐ₁₂)(Rₐ₁₃))ₙ₃-cycloalkylene)ₘ₄-;
wherein each Rₐ₇ independently represents H or C₁₋₃ alkyl;
the cycloalkylene (e.g., C₃₋₂₀cycloalkylene), arylene (e.g., C₅₋₂₀ arylene), heterocyclylene (e.g., 4-to 20-membered heterocyclylene), and heteroarylene (e.g., 5-to 20-membered heteroarylene) are each independently optionally substituted with a substituent selected from the group consisting of deuterium, hydroxy, amino, mercapto, nitro, halogen, cyano, oxo, C₁₋₆ alkyl, halogenated C₁₋₆ alkyl, deuterated C₁₋₆ alkyl, C₁₋₆ alkoxy, halogenated C₁₋₆ alkoxy, C₃₋₆ cycloalkyl, halogenated C₃₋₆ cycloalkyl, deuterated C₃₋₆ cycloalkyl, 4-to 10-membered heterocyclyl, and any combination thereof;
Rₐ₅, Rₐ₉, Rₐ₁₀, Rₐ₁₁, Rₐ₁₂, Rₐ₁₃, Rₐ₁₄, and Rₐ₁₅ each independently represent H, deuterium, hydroxy, amino, mercapto, nitro, halogen, cyano, oxo, halogenated C₁₋₆ alkyl, deuterated C₁₋₆ alkyl, C₁₋₆ alkoxy, halogenated C₁₋₆ alkoxy, C₃₋₆ cycloalkyl, halogenated C₃₋₆ cycloalkyl, deuterated C₃₋₆ cycloalkyl, 4-to 10-membered heterocyclyl, and any combination thereof, and
n1, n2, n3, n4, m3, m4, m5 each independently represent an integer of 1, 2, 3, 4, 5, 6, 7, 8, 9, or 10.

28. The compound of Formula (II) or salts, enantiomers, stereoisomers, solvates, isotopically enriched analogs, prodrugs, or polymorphs thereof as claimed in claim 21, wherein R_{w1} represents:
—CH₂—, —(CH₂)₂—, —(CH₂)₃—, —(CH₂)₄—, —(CH₂)₅—, —(CH₂)₆—, —(CH₂)₇—, —(CH₂)₈—, —(CH₂)₉—, —(CH₂)₁₀—, —(CH₂)₁₁—, —(CH₂)₁₂—, —(CH₂)₁₃—, —(CH₂)₁₄—, —(CH₂)₁₅—; —O—CH₂—, —O—(CH₂)₂—, —O—(CH₂)₃—, —O—(CH₂)₄—, —O—(CH₂)₅—, —O—(CH₂)₆—, —O—(CH₂)₇—, —O—(CH₂)₈—, —O—(CH₂)₉—, —O—(CH₂)₁₀—, —(CH₂)₁—O—, —(CH₂)₂—O—, —(CH₂)₃—O—, —(CH₂)₄—O—, —(CH₂)₅—O—, —(CH₂)₆—O—, —(CH₂)₇—O—, —(CH₂)₈—O—, —(CH₂)₉—O—, —(CH₂)₁₀—O—, —CH₂—O—CH₂—, —CH₂—O—(CH₂)₂—, —(CH₂)₁—O—(CH₂)₃—, —(CH₂)₁—O—(CH₂)₄—, —(CH₂)₁—O—(CH₂)₅—, —(CH₂)₁—O—(CH₂)₆—, —(CH₂)₁—O—(CH₂)₇—, —(CH₂)₁—O—(CH₂)₈—, —(CH₂)₁—O—(CH₂)₉—, —(CH₂)₁—O—(CH₂)₁₀—, —(CH₂)₂—O—(CH₂)₁—, —(CH₂)₂—O—(CH₂)₂—, —(CH₂)₂—O—(CH₂)₃—, —(CH₂)₂—O—(CH₂)₄—, —(CH₂)₂—O—(CH₂)₅—, —(CH₂)₂—O—(CH₂)₆—, —(CH₂)₂—O—(CH₂)₇—, —(CH₂)₂—O—(CH₂)₈—, —(CH₂)₂—O—(CH₂)₉—, —(CH₂)₂—O—(CH₂)₁₀—, —(CH₂)₃—O—(CH₂)₁—, —(CH₂)₃—O—(CH₂)₂—, —(CH₂)₃—O—(CH₂)₃—, —(CH₂)₃—O—(CH₂)₄—, —(CH₂)₃—O—(CH₂)₅—, —(CH₂)₃—O—(CH₂)₆—, —(CH₂)₃—O—(CH₂)₇—, —(CH₂)₄—O—(CH₂)₁—, —(CH₂)₄—O—(CH₂)₂—, —(CH₂)₄—O—(CH₂)₃—, —(CH₂)₄—O—(CH₂)₄—, —(CH₂)₄—O—(CH₂)₅—, —(CH₂)₄—O—(CH₂)₆—, —(CH₂)₅—O—(CH₂)₁—, —(CH₂)₅—O—(CH₂)₂—, —(CH₂)₅—O—(CH₂)₃—, —(CH₂)₅—O—(CH₂)₄—, —(CH₂)₅—O—(CH₂)₅—, —(CH₂)₆—O—(CH₂)₁—, —(CH₂)₆—O—(CH₂)₂—, —(CH₂)₆—O—(CH₂)₃—, —(CH₂)₆—O—(CH₂)₄—, —(CH₂)₇—O—(CH₂)₁—, —(CH₂)₇—O—(CH₂)₂—, —(CH₂)₇—O—(CH₂)₃—, —(CH₂)₈—O—(CH₂)₁—, —(CH₂)₈—O—(CH₂)₂—, —CH(CH₃)—O—(CH₂)₁—, —CH(CH₃)—O—(CH₂)₂—, —CH(CH₃)—O—(CH₂)₃—, —CH(CH₃)—O—(CH₂)₄—, —CH(CH₃)—O—(CH₂)₅—, —CH(CH₃)—O—(CH₂)₆—, —CH(CH₃)—O—(CH₂)₇—, —CH(CH₃)—O—(CH₂)₈—, —CH(CH₃)—O—(CH₂)₉—, —CH(CH₃)—O—(CH₂)₁₀—, —(O(CH₂)₂)₂—, —(O(CH₂)₂)₃—, —(O(CH₂)₂)₄—, —(O(CH₂)₂)₅—, —(O(CH₂)₂)₆—, —(O(CH₂)₂)₇—, —CH₂—(O(CH₂)₂)₂—, —CH₂—(O(CH₂)₂)₃—, —CH₂—(O(CH₂)₂)₄—, —CH₂—(O(CH₂)₂)₅—, —CH₂—(O(CH₂)₂)₆—, —CH₂—(O(CH₂)₂)₇—, —CH₂—(O(CH₂)₂)₁—OCH₂—, —CH₂—(O(CH₂)₂)₂—OCH₂—, —CH₂—(O(CH₂)₂)₃—OCH₂—, —CH₂—(O(CH₂)₂)₄—OCH₂—, —CH₂—(O(CH₂)₂)₅—OCH₂—, —(CH₂)₂—(O(CH₂)₂)₂—, —(CH₂)₂—(O(CH₂)₂)₃—, —(CH₂)₂—(O(CH₂)₂)₄—, —(CH₂)₂—(O(CH₂)₂)₅—, —(CH₂)₂—(O(CH₂)₂)₆—, —(CH₂)₃—(O(CH₂)₂)₂—, —(CH₂)₃—(O(CH₂)₂)₃—, —(CH₂)₃—(O(CH₂)₂)₄—, —(CH₂)₃—(O(CH₂)₂)₅—, —(CH₂)₄—(O(CH₂)₂)₂—, —(CH₂)₄—(O(CH₂)₂)₃—, —(CH₂)₄—(O(CH₂)₂)₄—, —(CH₂)₄—(O(CH₂)₂)₅—, —CH₂—(O(CH₂)₃)₂—, —CH₂—(O(CH₂)₃)₃—, —CH₂—(O(CH₂)₃)₄—, —(CH₂)₂—(O(CH₂)₃)₂—, —(CH₂)₂—(O(CH₂)₃)₃—, —(CH₂)₂—(O(CH₂)₃)₄—, —(CH₂)₃—(O(CH₂)₃)₂—, —(CH₂)₃—(O(CH₂)₃)₃—, —(CH₂)₃—(O(CH₂)₃)₄—, —CH₂—O—(CH₂)₂—O—(CH₂)₃—, —CH₂—(O(CH₂)₂)₂—(O(CH₂)₃)₂—, (CH₂)₂—O—(CH₂)₂—O—(CH₂)₃—, —(CH₂)₂—(O(CH₂)₂)₂—(O(CH₂)₃)₂—, —(CH₂)₃—O—(CH₂)₂—O—(CH₂)₃—, —(CH₂)₃—(O(CH₂)₂)₂—(O(CH₂)₃)₂—, —CH₂—O—(CH₂)₃—O—(CH₂)₂—, —CH₂—(O(CH₂)₃)₂—(O(CH₂)₂)₂—, —(CH₂)₂—O—(CH₂)₃—O—(CH₂)₂—, —(CH₂)₂—(O(CH₂)₃)₂—(O(CH₂)₂)₂—, —(CH₂)₃—O—(CH₂)₃—O—(CH₂)₂—, —(CH₂)₃—(O(CH₂)₃)₂—(O(CH₂)₂)₂—, —CH₂—O—(CH₂)₂—O—CH₂—, —(CH₂)₂—O—(CH₂)₂—O—CH₂—, —(CH₂)₂—(O(CH₂)₂)₂—O—(CH₂)₃—, —(CH₂)₂—(O(CH₂)₂)₃—O—(CH₂)₃—, —(CH₂)₂—(O(CH₂)₂)₄—O—(CH₂)₃—, —(CH₂)₅—(O(CH₂)₂)₂—O—(CH₂)₅—, —(CH₂)₅—(O(CH₂)₂)₂—O—(CH₂)₆—, —(CH₂)₁—N(Rₐ₇)—(CH₂)₁—, —(CH₂)₁—N(Rₐ₇)—(CH₂)₂—, —(CH₂)₁—N(Rₐ₇)—(CH₂)₃—, —(CH₂)₁—N(Rₐ₇)—(CH₂)₄—, —(CH₂)₁—N(Rₐ₇)—(CH₂)₅—, —(CH₂)₁—N(Rₐ₇)—(CH₂)₆—, —(CH₂)₁—N(Rₐ₇)—(CH₂)₇—, —(CH₂)₁—N(Rₐ₇)—(CH₂)₈—, —(CH₂)₁—N(Rₐ₇)—(CH₂)₉—, —(CH₂)₁—N(Rₐ₇)—(CH₂)₁₀—, —N(Rₐ₇)—(CH₂)₁—, —N(Rₐ₇)—(CH₂)₂—, —N(Rₐ₇)—(CH₂)₃—, —N(Rₐ₇)—(CH₂)₄—, —N(Rₐ₇)—(CH₂)₅—, —N(Rₐ₇)—(CH₂)₆—, —N(Rₐ₇)—(CH₂)₇—, —N(Rₐ₇)—(CH₂)₈—, —N(Rₐ₇)—(CH₂)₉—, —N(Rₐ₇)—(CH₂)₁₀—, —(CH₂)₂—N(Rₐ₇)—(CH₂)₁—, —(CH₂)₂—N(Rₐ₇)—(CH₂)₂—, —(CH₂)₂—N(Rₐ₇)—(CH₂)₃—, —(CH₂)₂—N(Rₐ₇)—(CH₂)₄—, —(CH₂)₂—N(Rₐ₇)—(CH₂)₅—, —(CH₂)₂—N(Rₐ₇)—(CH₂)₆—, —(CH₂)₂—N(Rₐ₇)—(CH₂)₇—, —(CH₂)₂—N(Rₐ₇)—(CH₂)₈—, —(CH₂)₂—N(Rₐ₇)—(CH₂)₉—, —(CH₂)₂—N(Rₐ₇)—(CH₂)₁₀—, —(CH₂)₃—N(Rₐ₇)—(CH₂)₁—, —(CH₂)₃—N(Rₐ₇)—(CH₂)₂—, —(CH₂)₃—N(Rₐ₇)—(CH₂)₃—, —(CH₂)₄—N(Rₐ₇)—(CH₂)₁—, —(CH₂)₄—N(Rₐ₇)—(CH₂)₂—, —(CH₂)₄—N(Rₐ₇)—(CH₂)₃—, —(CH₂)₄—N(Rₐ₇)—(CH₂)₄—, —(CH₂)₅—N(Rₐ₇)—(CH₂)₁—, —(CH₂)₅—N(Rₐ₇)—(CH₂)₂—, —(CH₂)₅—N(Rₐ₇)—(CH₂)₃—, —(CH₂)₅—N(Rₐ₇)—(CH₂)₄—, —(CH₂)₅—N(Rₐ₇)—(CH₂)₅—, —(CH₂)₆—N(Rₐ₇)—(CH₂)₁—, —(CH₂)₆—N(Rₐ₇)—(CH₂)₂—, —(CH₂)₆—N(Rₐ₇)—(CH₂)₃—, —(CH₂)₇—N(Rₐ₇)—(CH₂)₁—, —(CH₂)₇—N(Rₐ₇)—(CH₂)₂—, —(CH₂)₇—N(Rₐ₇)—(CH₂)₃—, —(CH₂)₈—N(Rₐ₇)—(CH₂)₁—, —(CH₂)₈—N(Rₐ₇)—(CH₂)₂—, —(CH₂)₈—N(Rₐ₇)—(CH₂)₃—, —CH(CH₃)—N(Rₐ₇)—(CH₂)₁—, —CH(CH₃)—N(Rₐ₇)—(CH₂)₂—, —CH(CH₃)—N(Rₐ₇)—(CH₂)₃—, —CH(CH₃)—N(Rₐ₇)—(CH₂)₄—, —CH(CH₃)—N(Rₐ₇)—(CH₂)₅—, —CH(CH₃)—N(Rₐ₇)—(CH₂)₆—, —CH(CH₃)—N(Rₐ₇)—(CH₂)₇—, —CH(CH₃)—N(Rₐ₇)—(CH₂)₈—, —CH(CH₃)—N(Rₐ₇)—(CH₂)₉—, —CH(CH₃)—N(Rₐ₇)—(CH₂)₁₀—, —C(O)NHCH₂—, —C(O)NH(CH₂)₂—, —C(O)NH(CH₂)₃—, —C(O)NH(CH₂)₄—, —C(O)NH(CH₂)₅—, —CH₂C(O)NHCH₂—, —(CH₂)₂C(O)NH(CH₂)₂—, —(CH₂)₂C(O)NH(CH₂)₃—, —(CH₂)₂C(O)NH(CH₂)₄—, —(CH₂)₂C(O)NH(CH₂)₅—, —(CH₂)₃C(O)NH(CH₂)₃—, —(CH₂)₃C(O)NH(CH₂)₄—, —(CH₂)₄C(O)NH(CH₂)₄—, —(CH₂)₅C(O)NH(CH₂)₅—, —(CH₂)₆C(O)NH(CH₂)₇—, —(CH₂)₆C(O)NH(CH₂)₆—, —(CH₂)₇C(O)NH(CH₂)₇—, —(CH₂)₂C(O)NH(CH₂)₂—O—(CH₂)₂—, —NHC(O)CH₂—, —NHC(O)(CH₂)₂—, —NHC(O)(CH₂)₃—, —NHC(O)(CH₂)₄—, —NHC(O)(CH₂)₅—, —CH₂NHC(O)CH₂—, —(CH₂)₂NHC(O)(CH₂)₂—, —(CH₂)₂NHC(O)(CH₂)₃—, —(CH₂)₂NHC(O)(CH₂)₄—, —(CH₂)₂NHC(O)(CH₂)₅—, —(CH₂)₃NHC(O)(CH₂)₃—, —(CH₂)₃NHC(O)(CH₂)₄—, —(CH₂)₄NHC(O)(CH₂)₄—, —(CH₂)₅NHC(O)(CH₂)₅—, —(CH₂)₆NHC(O)(CH₂)₇—, —(CH₂)₆NHC(O)(CH₂)₆—, —(CH₂)₇NHC(O)(CH₂)₇—, —(CH₂)₄NHC(O)(CH₂)₈—, —(CH₂)₂NHC(O)(CH₂)₂—O—(CH₂)₂—, —(CH₂)₄NHC(O)CH₂—,-phenylene-CH₂—,-phenylene-(CH₂)₂—,-phenylene-(CH₂)₃—,-phenylene-(CH₂)₄—,-phenylene-(CH₂)₅—,-phenylene-(CH₂)₆—,-phenylene-(CH₂)₇—,-phenylene-(CH₂)₈—, —CH₂-phenylene-CH₂—, —CH₂-phenylene-(CH₂)₂—, —CH₂-phenylene-(CH₂)₃—, —CH₂-phenylene-(CH₂)₄—, —CH₂-phenylene-(CH₂)₅—, —CH₂-phenylene-(CH₂)₆—, —CH₂-phenylene-(CH₂)₇—, —CH₂-phenylene-(CH₂)₈—, —(CH₂)₂-phenylene-(CH₂)₁—, —(CH₂)₂-phenylene-(CH₂)₂—, —(CH₂)₂-phenylene-(CH₂)₃—, —(CH₂)₂-phenylene-(CH₂)₄—, —(CH₂)₂-phenylene-(CH₂)₅—, —(CH₂)₂-phenylene-(CH₂)₆—, —(CH₂)₂-phenylene-(CH₂)₇—, —(CH₂)₂-phenylene-(CH₂)₈—, —(CH₂)₃-phenylene-CH₂—, —(CH₂)₃-phenylene-(CH₂)₂—, —(CH₂)₃-phenylene-(CH₂)₃—, —(CH₂)₃-phenylene-(CH₂)₄—, —(CH₂)₃-phenylene-(CH₂)₅—, —(CH₂)₃-phenylene-(CH₂)₆—, —(CH₂)₃-phenylene-(CH₂)₇—, —(CH₂)₃-phenylene-(CH₂)₈—, —(CH₂)₄-phenylene-CH₂—, —(CH₂)₄-phenylene-(CH₂)₂—, —(CH₂)₄-phenylene-(CH₂)₃—, —(CH₂)₄-phenylene-(CH₂)₄—, —(CH₂)₄-phenylene-(CH₂)₅—, —(CH₂)₄-phenylene-(CH₂)₆—, —(CH₂)₄-phenylene-(CH₂)₇—, —(CH₂)₄-phenylene-(CH₂)₈—, —(CH₂)₅-phenylene-(CH₂)₁—, —(CH₂)₅-phenylene-(CH₂)₂—, —(CH₂)₅-phenylene-(CH₂)₃—, —(CH₂)₅-phenylene-(CH₂)₄—, —(CH₂)₅-phenylene-(CH₂)₅—, —(CH₂)₅-phenylene-(CH₂)₆—, —(CH₂)₅-phenylene-(CH₂)₇—, —(CH₂)₅-phenylene-(CH₂)₈—, —(CH₂)₆-phenylene-(CH₂)₁—, —(CH₂)₆-phenylene-(CH₂)₂—, —(CH₂)₆-phenylene-(CH₂)₃—, —(CH₂)₆-phenylene-(CH₂)₄—, —(CH₂)₆-phenylene-(CH₂)₅—, —(CH₂)₆-phenylene-(CH₂)₆—, —(CH₂)₆-phenylene-(CH₂)₇—, —(CH₂)₆-phenylene-(CH₂)₈—, —(CH₂)₇-phenylene-(CH₂)₁—, —(CH₂)₇-phenylene-(CH₂)₂—, —(CH₂)₇-phenylene-(CH₂)₃—, —(CH₂)₇-phenylene-(CH₂)₄—, —(CH₂)₇-phenylene-(CH₂)₈—, —(CH₂)₈-phenylene-CH₂—, —(CH₂)₈-phenylene-(CH₂)₂—, —(CH₂)₈-phenylene-(CH₂)₃—, —(CH₂)₈-phenylene-(CH₂)₄—, —(CH₂)₈-phenylene-(CH₂)₅—, —(CH₂)₈-phenylene-(CH₂)₆—, —(CH₂)₈-phenylene-(CH₂)₇—, —N(Rₐ₇)—CH₂-phenylene-CH₂—, —N(Rₐ₇)—CH₂-phenylene-(CH₂)₂—, —N(Rₐ₇)—CH₂-phenylene-(CH₂)₃—, —N(Rₐ₇)—CH₂-phenylene-(CH₂)₄—, —N(Rₐ₇)—CH₂-phenylene-(CH₂)₅—, —N(Rₐ₇)—CH₂-phenylene-(CH₂)₆—, —N(Rₐ₇)—CH₂-phenylene-(CH₂)₇—, —N(Rₐ₇)—CH₂-phenylene-(CH₂)₈—, —CH₂—N(Rₐ₇)—CH₂-phenylene-CH₂—, —CH₂—N(Rₐ₇)—CH₂-phenylene-(CH₂)₂—, —CH₂—N(Rₐ₇)—CH₂-phenylene-(CH₂)₃—, —CH₂—N(Rₐ₇)—CH₂-phenylene-(CH₂)₄—, —CH₂—N(Rₐ₇)—CH₂-phenylene-(CH₂)₅—, —CH₂—N(Rₐ₇)—CH₂-phenylene-(CH₂)₆—, —CH₂—N(Rₐ₇)—CH₂-phenylene-(CH₂)₇—, —CH₂—N(Rₐ₇)—CH₂-phenylene-(CH₂)₈—, —CH₂—N(Rₐ₇)—(CH₂)₂-phenylene-CH₂—, —CH₂—N(Rₐ₇)—(CH₂)₂-phenylene-(CH₂)₂—, —CH₂—N(Rₐ₇)—(CH₂)₂-phenylene-(CH₂)₃—, —CH₂—N(Rₐ₇)—(CH₂)₂-phenylene-(CH₂)₄—, —CH₂—N(Rₐ₇)—(CH₂)₂-phenylene-(CH₂)₅—, —CH₂—N(Rₐ₇)—(CH₂)₂-phenylene-(CH₂)₆—, —CH₂—N(Rₐ₇)—(CH₂)₂-phenylene-(CH₂)₇—, —CH₂—N(Rₐ₇)—(CH₂)₂-phenylene-(CH₂)₈—, —(CH₂)₂—N(Rₐ₇)—CH₂-phenylene-CH₂—, —(CH₂)₂—N(Rₐ₇)—CH₂-phenylene-(CH₂)₂—, —(CH₂)₂—N(Rₐ₇)—CH₂-phenylene-(CH₂)₃—, —(CH₂)₂—N(Rₐ₇)—CH₂-phenylene-(CH₂)₄—, —(CH₂)₂—N(Rₐ₇)—CH₂-phenylene-(CH₂)₅—, —(CH₂)₂—N(Rₐ₇)—CH₂-phenylene-(CH₂)₆—, —(CH₂)₂—N(Rₐ₇)—CH₂-phenylene-(CH₂)₇—, —(CH₂)₂—N(Rₐ₇)—CH₂-phenylene-(CH₂)₈—, —(CH₂)₃—N(Rₐ₇)—CH₂-phenylene-CH₂—, —(CH₂)₃—N(Rₐ₇)—CH₂-phenylene-(CH₂)₂—, —(CH₂)₃—N(Rₐ₇)—CH₂-phenylene-(CH₂)₃—, —(CH₂)₃—N(Rₐ₇)—CH₂-phenylene-(CH₂)₈—, —(CH₂)₄—N(Rₐ₇)—CH₂-phenylene-CH₂—, —(CH₂)₄—N(Rₐ₇)—CH₂-phenylene-(CH₂)₂—, —(CH₂)₄—N(Rₐ₇)—CH₂-phenylene-(CH₂)₃—, —(CH₂)₄—N(Rₐ₇)—CH₂-phenylene-(CH₂)₈—, —(CH₂)₅—N(Rₐ₇)—CH₂-phenylene-(CH₂)₃—, —(CH₂)₅—N(Rₐ₇)—CH₂-phenylene-(CH₂)₈—, —(CH₂)₆—N(Rₐ₇)—CH₂-phenylene-(CH₂)₃—, —(CH₂)₆—N(Rₐ₇)—CH₂-phenylene-(CH₂)₈—, —(CH₂)₇—N(Rₐ₇)—CH₂-phenylene-(CH₂)₃—, —(CH₂)₈—N(Rₐ₇)—CH₂-phenylene-CH₂—, —(CH₂)₈—N(Rₐ₇)—CH₂-phenylene-(CH₂)₂—, —(CH₂)₈—N(Rₐ₇)—CH₂-phenylene-(CH₂)₃—, —(CH₂)₈—N(Rₐ₇)—CH₂-phenylene-(CH₂)₄—,
(CH₂)₈—N(Rₐ₇)—CH₂-phenylene-(CH₂)₅—, —(CH₂)₈—N(Rₐ₇)—CH₂-phenylene-(CH₂)₆—,-piperidinylene-CH₂—,- piperidinylene-(CH₂)₂—,-piperidinylene-(CH₂)₃—,-piperidinylene-(CH₂)₄—,-piperidinylene-(CH₂)₅—,-piperidinylene-(CH₂)₆—,-piperidinylene-(CH₂)₇—,-piperidinylene-(CH₂)₈—, —CH₂-piperidinylene-CH₂—, —CH₂-piperidinylene-(CH₂)₂—, —CH₂-piperidinylene-(CH₂)₃—, —CH₂-piperidinylene-(CH₂)₄—, —CH₂-piperidinylene-(CH₂)₅—, —CH₂-piperidinylene-(CH₂)₆—, —CH₂-piperidinylene-(CH₂)₇—, —CH₂-piperidinylene-(CH₂)₈—, —(CH₂)₂-piperidinylene-(CH₂)₁—, —(CH₂)₂-piperidinylene-(CH₂)₂—, —(CH₂)₂-piperidinylene-(CH₂)₃—, —(CH₂)₂-piperidinylene-(CH₂)₄—, —(CH₂)₂-piperidinylene-(CH₂)₅—, —(CH₂)₂-piperidinylene-(CH₂)₆—, —(CH₂)₂-piperidinylene-(CH₂)₇—, —(CH₂)₂-piperidinylene-(CH₂)₈—, —(CH₂)₃-piperidinylene-CH₂—, —(CH₂)₃-piperidinylene-(CH₂)₂—, —(CH₂)₃-piperidinylene-(CH₂)₃—, —(CH₂)₃-piperidinylene-(CH₂)₄—, —(CH₂)₃-piperidinylene-(CH₂)₅—, —(CH₂)₃-piperidinylene-(CH₂)₆—, —(CH₂)₃-piperidinylene-(CH₂)₇—, —(CH₂)₃-piperidinylene-(CH₂)₈—, —(CH₂)₄-piperidinylene-CH₂—, —(CH₂)₄-piperidinylene-(CH₂)₂—, —(CH₂)₄-piperidinylene-(CH₂)₃—, —(CH₂)₄-piperidinylene-(CH₂)₄—, —(CH₂)₄-piperidinylene-(CH₂)₅—, —(CH₂)₄-piperidinylene-(CH₂)₆—, —(CH₂)₄-piperidinylene-(CH₂)₇—, —(CH₂)₄-piperidinylene-(CH₂)₈—, —(CH₂)₅-piperidinylene-(CH₂)₁—, —(CH₂)₅-piperidinylene-(CH₂)₂—, —(CH₂)₅-piperidinylene-(CH₂)₃—, —(CH₂)₅-piperidinylene-(CH₂)₄—, —(CH₂)₅-piperidinylene-(CH₂)₅—, —(CH₂)₅-piperidinylene-(CH₂)₆—, —(CH₂)₅-piperidinylene-(CH₂)₇—, —(CH₂)₅-piperidinylene-(CH₂)₈—, —(CH₂)₆-piperidinylene-(CH₂)₁—, —(CH₂)₆-piperidinylene-(CH₂)₂—, —(CH₂)₆-piperidinylene-(CH₂)₃—, —(CH₂)₆-piperidinylene-(CH₂)₄—, —(CH₂)₆-piperidinylene-(CH₂)₅—, —(CH₂)₆-piperidinylene-(CH₂)₆—, —(CH₂)₆-piperidinylene-(CH₂)₇—, —(CH₂)₆-piperidinylene-(CH₂)₈—, —(CH₂)₇-piperidinylene-(CH₂)₁—, —(CH₂)₇-piperidinylene-(CH₂)₂—, —(CH₂)₇-piperidinylene-(CH₂)₃—, —(CH₂)₇-piperidinylene-(CH₂)₄—, —(CH₂)₇-piperidinylene-(CH₂)₈—, —(CH₂)₈-piperidinylene-CH₂—, —(CH₂)₈-piperidinylene-(CH₂)₂—, —(CH₂)₈-piperidinylene-(CH₂)₃—, —(CH₂)₈-piperidinylene-(CH₂)₄—, —(CH₂)₈-piperidinylene-(CH₂)₅—, —(CH₂)₈-piperidinylene-(CH₂)₆—, —(CH₂)₈-piperidinylene-(CH₂)₇—, —N(Rₐ₇)—CH₂-piperidinylene-CH₂—, —N(Rₐ₇)—CH₂-piperidinylene-(CH₂)₂—, —N(Rₐ₇)—CH₂-piperidinylene-(CH₂)₃—, —N(Rₐ₇)—CH₂-piperidinylene-(CH₂)₄—, —N(Rₐ₇)—CH₂-piperidinylene-(CH₂)₅—, —N(Rₐ₇)—CH₂-piperidinylene-(CH₂)₆—, —N(Rₐ₇)—CH₂-piperidinylene-(CH₂)₇—, —N(Rₐ₇)—CH₂-piperidinylene-(CH₂)₈—, —CH₂—N(Rₐ₇)—CH₂-piperidinylene-CH₂—, —CH₂—N(Rₐ₇)—CH₂-piperidinylene-(CH₂)₂—, —CH₂—N(Rₐ₇)—CH₂-piperidinylene-(CH₂)₃—, —CH₂—N(Rₐ₇)—CH₂-piperidinylene-(CH₂)₄—, —CH₂—N(Rₐ₇)—CH₂-piperidinylene-(CH₂)₅—, —CH₂—N(Rₐ₇)—CH₂-piperidinylene-(CH₂)₆—, —CH₂—N(Rₐ₇)—CH₂-piperidinylene-(CH₂)₇—, —CH₂—N(Rₐ₇)—CH₂-piperidinylene-(CH₂)₈—, —CH₂—N(Rₐ₇)—(CH₂)₂-piperidinylene-CH₂—, —CH₂—N(Rₐ₇)—(CH₂)₂-piperidinylene-(CH₂)₂—, —CH₂—N(Rₐ₇)—(CH₂)₂-piperidinylene-(CH₂)₃—, —CH₂—N(Rₐ₇)—(CH₂)₂-piperidinylene-(CH₂)₄—, —CH₂—N(Rₐ₇)—(CH₂)₂-piperidinylene-(CH₂)₅—, —CH₂—N(Rₐ₇)—(CH₂)₂-piperidinylene-(CH₂)₆—, —CH₂—N(Rₐ₇)—(CH₂)₂-piperidinylene-(CH₂)₇—, —CH₂—N(Rₐ₇)—(CH₂)₂-piperidinylene-(CH₂)₈—, —(CH₂)₂—N(Rₐ₇)—CH₂-piperidinylene-CH₂—, —(CH₂)₂—N(Rₐ₇)—CH₂-piperidinylene-(CH₂)₂—, —(CH₂)₂—N(Rₐ₇)—CH₂-piperidinylene-(CH₂)₃—, —(CH₂)₂—N(Rₐ₇)—CH₂-piperidinylene-(CH₂)₄—, (CH₂)₂—N(Rₐ₇)—CH₂-piperidinylene-(CH₂)₅—, —(CH₂)₂—N(Rₐ₇)—CH₂-piperidinylene-(CH₂)₆—, —(CH₂)₂—N(Rₐ₇)—CH₂-piperidinylene-(CH₂)₇—, —(CH₂)₂—N(Rₐ₇)—CH₂-piperidinylene-(CH₂)₈—, —(CH₂)₃—N(Rₐ₇)—CH₂-piperidinylene-CH₂—, —(CH₂)₃—N(Rₐ₇)—CH₂-piperidinylene-(CH₂)₂—, —(CH₂)₃—N(Rₐ₇)—CH₂-piperidinylene-(CH₂)₃—, —(CH₂)₃—N(Rₐ₇)—CH₂-piperidinylene-(CH₂)₈—, —(CH₂)₄—N(Rₐ₇)—CH₂-piperidinylene-CH₂—, —(CH₂)₄—N(Rₐ₇)—CH₂-piperidinylene-(CH₂)₂—, —(CH₂)₄—N(Rₐ₇)—CH₂-piperidinylene-(CH₂)₃—, —(CH₂)₄—N(Rₐ₇)—CH₂-piperidinylene-(CH₂)₈—, —(CH₂)₅—N(Rₐ₇)—CH₂-piperidinylene-(CH₂)₃—, —(CH₂)₅—N(Rₐ₇)—CH₂-piperidinylene-(CH₂)₈—, —(CH₂)₆—N(Rₐ₇)—CH₂-piperidinylene-(CH₂)₃—, (CH₂)₆—N(Rₐ₇)—CH₂-piperidinylene-(CH₂)₈—, —(CH₂)₇—N(Rₐ₇)—CH₂-piperidinylene-(CH₂)₃—, —(CH₂)₈—N(Rₐ₇)—CH₂-piperidinylene-CH₂—, —(CH₂)₈—N(Rₐ₇)—CH₂-piperidinylene-(CH₂)₂—, —(CH₂)₈—N(Rₐ₇)—CH₂-piperidinylene-(CH₂)₃—, —(CH₂)₈—N(Rₐ₇)—CH₂-piperidinylene-(CH₂)₄—, —(CH₂)₈—N(Rₐ₇)—CH₂-piperidinylene-(CH₂)₅—, —(CH₂)₈—N(Rₐ₇)—CH₂-piperidinylene-(CH₂)₆—,-piperazinylene-CH₂—,-piperazinylene-(CH₂)₂—,-piperazinylene-(CH₂)₃—,-piperazinylene-(CH₂)₄—,-piperazinylene-(CH₂)₅—,-piperazinylene-(CH₂)₆—,-piperazinylene-(CH₂)₇—,-piperazinylene-(CH₂)₈—, —CH₂-piperazinylene-CH₂—, —CH₂-piperazinylene-(CH₂)₂—, —CH₂-piperazinylene-(CH₂)₃—, —CH₂-piperazinylene-(CH₂)₄—, —CH₂-piperazinylene-(CH₂)₅—, —CH₂-piperazinylene-(CH₂)₆—, —CH₂-piperazinylene-(CH₂)₇—, —CH₂-piperazinylene-(CH₂)₈—, —(CH₂)₂-piperazinylene-(CH₂)₁—, —(CH₂)₂-piperazinylene-(CH₂)₂—, —(CH₂)₂-piperazinylene-(CH₂)₃—, —(CH₂)₂-piperazinylene-(CH₂)₄—, —(CH₂)₂-piperazinylene-(CH₂)₅—, —(CH₂)₂-piperazinylene-(CH₂)₆—, —(CH₂)₂-piperazinylene-(CH₂)₇—, —(CH₂)₂-piperazinylene-(CH₂)₈—, —(CH₂)₃-piperazinylene-CH₂—, —(CH₂)₃-piperazinylene-(CH₂)₂—, —(CH₂)₃-piperazinylene-(CH₂)₃—, —(CH₂)₃-piperazinylene-(CH₂)₄—, —(CH₂)₃-piperazinylene-(CH₂)₅—, —(CH₂)₃-piperazinylene-(CH₂)₆—, —(CH₂)₃-piperazinylene-(CH₂)₇—, —(CH₂)₃-piperazinylene-(CH₂)₈—, —(CH₂)₄-piperazinylene-CH₂—, —(CH₂)₄-piperazinylene-(CH₂)₂—, —(CH₂)₄-piperazinylene-(CH₂)₃—, —(CH₂)₄-piperazinylene-(CH₂)₄—, —(CH₂)₄-piperazinylene-(CH₂)₅—, —(CH₂)₄-piperazinylene-(CH₂)₆—, —(CH₂)₄-piperazinylene-(CH₂)₇—, —(CH₂)₄-piperazinylene-(CH₂)₈—, —(CH₂)₅-piperazinylene-(CH₂)₁—, —(CH₂)₅-piperazinylene-(CH₂)₂—, —(CH₂)₅-piperazinylene-(CH₂)₃—, —(CH₂)₅-piperazinylene-(CH₂)₄—, —(CH₂)₅-piperazinylene-(CH₂)₅—, —(CH₂)₅-piperazinylene-(CH₂)₆—, —(CH₂)₅-piperazinylene-(CH₂)₇—, —(CH₂)₅-piperazinylene-(CH₂)₈—, —(CH₂)₆-piperazinylene-(CH₂)₁—, —(CH₂)₆-piperazinylene-(CH₂)₂—, —(CH₂)₆-piperazinylene-(CH₂)₃—, —(CH₂)₆-piperazinylene-(CH₂)₄—, —(CH₂)₆-piperazinylene-(CH₂)₅—, —(CH₂)₆-piperazinylene-(CH₂)₆—, —(CH₂)₆-piperazinylene-(CH₂)₇—, —(CH₂)₆-piperazinylene-(CH₂)₈—, —(CH₂)₇-piperazinylene-(CH₂)₁—, —(CH₂)₇-piperazinylene-(CH₂)₂—, —(CH₂)₇-piperazinylene-(CH₂)₃—, —(CH₂)₇-piperazinylene-(CH₂)₄—, —(CH₂)₈-piperazinylene-CH₂—, —(CH₂)₈-piperazinylene-(CH₂)₂—, —(CH₂)₈-piperazinylene-(CH₂)₃—, —(CH₂)₈-piperazinylene-(CH₂)₄—, —(CH₂)₈-piperazinylene-(CH₂)₅—, or —(CH₂)₈-piperazinylene-(CH₂)₆—;
wherein each Rₐ₇ independently represents H or C₁₋₃ alkyl;
the phenylene, piperazinylene and piperidinylene are each independently optionally substituted with a substituent selected from the group consisting of deuterium, hydroxy, amino, mercapto, nitro, halogen, cyano, oxo, C₁₋₆ alkyl, halogenated C₁₋₆ alkyl, deuterated C₁₋₆ alkyl, C₁₋₆ alkoxy, halogenated C₁₋₆ alkoxy, C₃₋₆ cycloalkyl, halogenated C₃₋₆ cycloalkyl, deuterated C₃₋₆ cycloalkyl, 4-to 10-membered heterocyclyl, and any combination thereof.

29. The compound of Formula (I) or salts, enantiomers, stereoisomers, solvates, isotopically enriched analogs, prodrugs, or polymorphs thereof as claimed in claim 21, which is selected from:
1-(1-oxoisoindolin-2-yl)dihydropyrimidine-2,4 (1H,3H)-dione;
2-(2,4-dioxotetrahydropyrimidin-1 (2H)-yl)-5-fluoroisoindoline-1,3-dione;
2-(2,4-dioxotetrahydropyrimidin-1 (2H)-yl)-4-fluoroisoindoline-1,3-dione;
2-(2,4-dioxotetrahydropyrimidin-1 (2H)-yl)-4-hydroxyisoindoline-1,3-dione;
2-(2,4-dioxotetrahydropyrimidin-1 (2H)-yl)-5-hydroxyisoindoline-1,3-dione;
5-bromo-2-(2,4-dioxotetrahydropyrimidin-1 (2H)-yl)isoindoline-1,3-dione;
4-bromo-2-(2,4-dioxotetrahydropyrimidin-1 (2H)-yl)isoindoline-1,3-dione;
2-(2,4-dioxotetrahydropyrimidin-1 (2H)-yl)-5-(hydroxymethyl)isoindoline-1,3-dione;
2-(2,4-dioxotetrahydropyrimidin-1 (2H)-yl)-4-(hydroxymethyl)isoindoline-1,3-dione;
5-(aminomethyl)-2-(2,4-dioxotetrahydropyrimidin-1 (2H)-yl)isoindoline-1,3-dione;
5-(2-aminoethyl)-2-(2,4-dioxotetrahydropyrimidin-1 (2H)-yl)isoindoline-1,3-dione;
5-(3-aminopropyl)-2-(2,4-dioxotetrahydropyrimidin-1 (2H)-yl)isoindoline-1,3-dione;
4-(aminomethyl)-2-(2,4-dioxotetrahydropyrimidin-1 (2H)-yl)isoindoline-1,3-dione;
4-(2-aminoethyl)-2-(2,4-dioxotetrahydropyrimidin-1 (2H)-yl)isoindoline-1,3-dione;
4-(3-aminopropyl)-2-(2,4-dioxotetrahydropyrimidin-1 (2H)-yl)isoindoline-1,3-dione;
2-(2,4-dioxotetrahydropyrimidin-1 (2H)-yl)-5-(iodomethyl)isoindoline-1,3-dione;
2-(2,4-dioxotetrahydropyrimidin-1 (2H)-yl)-5-(2-iodoethyl)isoindoline-1,3-dione;
2-(2,4-dioxotetrahydropyrimidin-1 (2H)-yl)-5-(3-iodopropyl)isoindoline-1,3-dione;
2-(2,4-dioxotetrahydropyrimidin-1 (2H)-yl)-4-(iodomethyl)isoindoline-1,3-dione;
2-(2,4-dioxotetrahydropyrimidin-1 (2H)-yl)-4-(2-iodoethyl)isoindoline-1,3-dione;
2-(2,4-dioxotetrahydropyrimidin-1 (2H)-yl)-4-(3-iodopropyl)isoindoline-1,3-dione;
5-(bromomethyl)-2-(2,4-dioxotetrahydropyrimidin-1 (2H)-yl)isoindoline-1,3-dione;
5-(2-bromoethyl)-2-(2,4-dioxotetrahydropyrimidin-1 (2H)-yl)isoindoline-1,3-dione;
5-(3-bromopropyl)-2-(2,4-dioxotetrahydropyrimidin-1 (2H)-yl)isoindoline-1,3-dione;
4-(bromomethyl)-2-(2,4-dioxotetrahydropyrimidin-1 (2H)-yl)isoindoline-1,3-dione;
4-(2-bromoethyl)-2-(2,4-dioxotetrahydropyrimidin-1 (2H)-yl)isoindoline-1,3-dione;
4-(3-bromopropyl)-2-(2,4-dioxotetrahydropyrimidin-1 (2H)-yl)isoindoline-1,3-dione;
5-(chloromethyl)-2-(2,4-dioxotetrahydropyrimidin-1 (2H)-yl)isoindoline-1,3-dione;
5-(2-chloroethyl)-2-(2,4-dioxotetrahydropyrimidin-1 (2H)-yl)isoindoline-1,3-dione;
5-(3-chloropropyl)-2-(2,4-dioxotetrahydropyrimidin-1 (2H)-yl)isoindoline-1,3-dione;
4-(chloromethyl)-2-(2,4-dioxotetrahydropyrimidin-1 (2H)-yl)isoindoline-1,3-dione;
4-(2-chloroethyl)-2-(2,4-dioxotetrahydropyrimidin-1 (2H)-yl)isoindoline-1,3-dione;
4-(3-chloropropyl)-2-(2,4-dioxotetrahydropyrimidin-1 (2H)-yl)isoindoline-1,3-dione;
(2-(2,4-dioxotetrahydropyrimidin-1 (2H)-yl)-1,3-dioxoisoindolin-5-yl)methyl 4-methylbenzenesulfonate;
2-(2-(2,4-dioxotetrahydropyrimidin-1 (2H)-yl)-1,3-dioxoisoindolin-5-yl)ethyl 4-methylbenzenesulfonate;
3-(2-(2,4-dioxotetrahydropyrimidin-1 (2H)-yl)-1,3-dioxoisoindolin-5-yl) propyl 4-methylbenzenesulfonate;
(2-(2,4-dioxotetrahydropyrimidin-1 (2H)-yl)-1,3-dioxoisoindolin-4-yl)methyl 4-methylbenzenesulfonate;
2-(2-(2,4-dioxotetrahydropyrimidin-1 (2H)-yl)-1,3-dioxoisoindolin-4-yl)ethyl 4-methylbenzenesulfonate;
3-(2-(2,4-dioxotetrahydropyrimidin-1 (2H)-yl)-1,3-dioxoisoindolin-4-yl) propyl 4-methylbenzenesulfonate;
2-(2,4-dioxotetrahydropyrimidin-1 (2H)-yl)-4-(piperazin-1-yl)isoindoline-1,3-dione;
2-(2,4-dioxotetrahydropyrimidin-1 (2H)-yl)-5-(piperazin-1-yl)isoindoline-1,3-dione;
2-(2,4-dioxotetrahydropyrimidin-1 (2H)-yl)-4-fluoro-5-(piperazin-1-yl)isoindoline-1,3-dione;
2-(2,4-dioxotetrahydropyrimidin-1 (2H)-yl)-5-fluoro-6-(piperazin-1-yl)isoindoline-1,3-dione;
2-(2,4-dioxotetrahydropyrimidin-1 (2H)-yl)-4-fluoro-6-(piperazin-1-yl)isoindoline-1,3-dione;
5-(3,6-diazabicyclo[3.1.1]heptan-3-yl)-2-(2,4-dioxotetrahydropyrimidin-1 (2H)-yl)isoindoline-1,3-dione;
5-(3,6-diazabicyclo[3.1.1]heptan-6-yl)-2-(2,4-dioxotetrahydropyrimidin-1 (2H)-yl)isoindoline-1,3-dione;
5-((1R,4R)-2,5-diazabicyclo[2.2.1]heptan-2-yl)-2-(2,4-dioxotetrahydropyrimidin-1 (2H)-yl)isoindoline-1,3-dione;
5-((1S,4S)-2,5-diazabicyclo[2.2.1]heptan-2-yl)-2-(2,4-dioxotetrahydropyrimidin-1 (2H)-yl)isoindoline-1,3-dione;
5-(3,8-diazabicyclo[3.2.1]octan-3-yl)-2-(2,4-dioxotetrahydropyrimidin-1 (2H)-yl)isoindoline-1,3-dione;
5-(3,8-diazabicyclo[3.2.1]octan-8-yl)-2-(2,4-dioxotetrahydropyrimidin-1 (2H)-yl)isoindoline-1,3-dione;
5-(2,5-diazabicyclo[2.2.2]octan-2-yl)-2-(2,4-dioxotetrahydropyrimidin-1 (2H)-yl)isoindoline-1,3-dione;
2-(2,4-dioxotetrahydropyrimidin-1 (2H)-yl)-5-(4-(piperazin-1-yl)piperidin-1-yl)isoindoline-1,3-dione;
5-(3,3-difluoro-4-(piperazin-1-yl)piperidin-1-yl)-2-(2,4-dioxotetrahydropyrimidin-1 (2H)-yl)isoindoline-1,3-dione;
1-(2-(2,4-dioxotetrahydropyrimidin-1 (2H)-yl)-1,3-dioxoisoindolin-4-yl)piperidine-4-carbaldehyde;
1-(2-(2,4-dioxotetrahydropyrimidin-1 (2H)-yl)-1,3-dioxoisoindolin-5-yl)piperidine-4-carbaldehyde;
1′-(2-(2,4-dioxotetrahydropyrimidin-1 (2H)-yl)-1,3-dioxoisoindolin-5-yl)-[1,4′-bipiperidine]-4-carbaldehyde;
1-(5-fluoro-1-oxoisoindolin-2-yl)dihydropyrimidine-2,4 (1H,3H)-dione;
1-(4-fluoro-1-oxoisoindolin-2-yl)dihydropyrimidine-2,4 (1H,3H)-dione;
1-(6-fluoro-1-oxoisoindolin-2-yl)dihydropyrimidine-2,4 (1H,3H)-dione;
1-(7-fluoro-1-oxoisoindolin-2-yl)dihydropyrimidine-2,4 (1H,3H)-dione;
1-(4-hydroxy-1-oxoisoindolin-2-yl)dihydropyrimidine-2,4 (1H,3H)-dione;
1-(5-hydroxy-1-oxoisoindolin-2-yl)dihydropyrimidine-2,4 (1H,3H)-dione;
1-(6-hydroxy-1-oxoisoindolin-2-yl)dihydropyrimidine-2,4 (1H,3H)-dione;
1-(7-hydroxy-1-oxoisoindolin-2-yl)dihydropyrimidine-2,4 (1H,3H)-dione;
1-(5-bromo-1-oxoisoindolin-2-yl)dihydropyrimidine-2,4 (1H,3H)-dione;
1-(4-bromo-1-oxoisoindolin-2-yl)dihydropyrimidine-2,4 (1H,3H)-dione;
1-(5-bromo-1-oxoisoindolin-2-yl)dihydropyrimidine-2,4 (1H,3H)-dione;
1-(7-bromo-1-oxoisoindolin-2-yl)dihydropyrimidine-2,4 (1H,3H)-dione;
1-(5-(hydroxymethyl)-1-oxoisoindolin-2-yl)dihydropyrimidine-2,4 (1H,3H)-dione;
1-(4-(hydroxymethyl)-1-oxoisoindolin-2-yl)dihydropyrimidine-2,4 (1H,3H)-dione;
1-(5-(aminomethyl)-1-oxoisoindolin-2-yl)dihydropyrimidine-2,4 (1H,3H)-dione;
1-(5-(2-aminoethyl)-1-oxoisoindolin-2-yl)dihydropyrimidine-2,4 (1H,3H)-dione;
1-(5-(3-aminopropyl)-1-oxoisoindolin-2-yl)dihydropyrimidine-2,4 (1H,3H)-dione;
1-(4-(aminomethyl)-1-oxoisoindolin-2-yl)dihydropyrimidine-2,4 (1H,3H)-dione;
1-(4-(2-aminoethyl)-1-oxoisoindolin-2-yl)dihydropyrimidine-2,4 (1H,3H)-dione;
1-(4-(3-aminopropyl)-1-oxoisoindolin-2-yl)dihydropyrimidine-2,4 (1H,3H)-dione;
1-(5-(iodomethyl)-1-oxoisoindolin-2-yl)dihydropyrimidine-2,4 (1H,3H)-dione;
1-(5-(2-iodoethyl)-1-oxoisoindolin-2-yl)dihydropyrimidine-2,4 (1H,3H)-dione;
1-(5-(3-iodopropyl)-1-oxoisoindolin-2-yl)dihydropyrimidine-2,4 (1H,3H)-dione;
1-(4-(iodomethyl)-1-oxoisoindolin-2-yl)dihydropyrimidine-2,4 (1H,3H)-dione;
1-(4-(2-iodoethyl)-1-oxoisoindolin-2-yl)dihydropyrimidine-2,4 (1H,3H)-dione;
1-(4-(3-iodopropyl)-1-oxoisoindolin-2-yl)dihydropyrimidine-2,4 (1H,3H)-dione;
1-(5-(bromomethyl)-1-oxoisoindolin-2-yl)dihydropyrimidine-2,4 (1H,3H)-dione;
1-(5-(2-bromoethyl)-1-oxoisoindolin-2-yl)dihydropyrimidine-2,4 (1H,3H)-dione;
1-(5-(3-bromopropyl)-1-oxoisoindolin-2-yl)dihydropyrimidine-2,4 (1H,3H)-dione;
1-(4-(bromomethyl)-1-oxoisoindolin-2-yl)dihydropyrimidine-2,4 (1H,3H)-dione;
1-(4-(2-bromoethyl)-1-oxoisoindolin-2-yl)dihydropyrimidine-2,4 (1H,3H)-dione;
1-(4-(3-bromopropyl)-1-oxoisoindolin-2-yl)dihydropyrimidine-2,4 (1H,3H)-dione;
1-(5-(chloromethyl)-1-oxoisoindolin-2-yl)dihydropyrimidine-2,4 (1H,3H)-dione;
1-(5-(2-chloroethyl)-1-oxoisoindolin-2-yl)dihydropyrimidine-2,4 (1H,3H)-dione;
1-(5-(3-chloropropyl)-1-oxoisoindolin-2-yl)dihydropyrimidine-2,4 (1H,3H)-dione;
1-(4-(chloromethyl)-1-oxoisoindolin-2-yl)dihydropyrimidine-2,4 (1H,3H)-dione;
1-(4-(2-chloroethyl)-1-oxoisoindolin-2-yl)dihydropyrimidine-2,4 (1H,3H)-dione;
1-(4-(3-chloropropyl)-1-oxoisoindolin-2-yl)dihydropyrimidine-2,4 (1H,3H)-dione;
(2-(2,4-dioxotetrahydropyrimidin-1 (2H)-yl)-1-oxoisoindolin-5-yl)methyl 4-methylbenzenesulfonate;
2-(2-(2,4-dioxotetrahydropyrimidin-1 (2H)-yl)-1-oxoisoindolin-5-yl)ethyl 4-methylbenzenesulfonate;
3-(2-(2,4-dioxotetrahydropyrimidin-1 (2H)-yl)-1-oxoisoindolin-5-yl) propyl 4-methylbenzenesulfonate;
(2-(2,4-dioxotetrahydropyrimidin-1 (2H)-yl)-1-oxoisoindolin-4-yl)methyl 4-methylbenzenesulfonate;
2-(2-(2,4-dioxotetrahydropyrimidin-1 (2H)-yl)-1-oxoisoindolin-4-yl)ethyl 4-methylbenzenesulfonate;
3-(2-(2,4-dioxotetrahydropyrimidin-1 (2H)-yl)-1-oxoisoindolin-4-yl) propyl 4-methylbenzenesulfonate;
1-(1-oxo-4-(piperazin-1-yl)isoindolin-2-yl)dihydropyrimidine-2,4 (1H,3H)-dione;
1-(1-oxo-5-(piperazin-1-yl)isoindolin-2-yl)dihydropyrimidine-2,4 (1H,3H)-dione;
1-(4-fluoro-1-oxo-5-(piperazin-1-yl)isoindolin-2-yl)dihydropyrimidine-2,4 (1H,3H)-dione;
1-(6-fluoro-1-oxo-5-(piperazin-1-yl)isoindolin-2-yl)dihydropyrimidine-2,4 (1H,3H)-dione;
1-(7-fluoro-1-oxo-5-(piperazin-1-yl)isoindolin-2-yl)dihydropyrimidine-2,4 (1H,3H)-dione;
1-(5-(3,6-diazabicyclo[3.1.1]heptan-3-yl)-1-oxoisoindolin-2-yl)dihydropyrimidine-2,4 (1H,3H)-dione;
1-(5-(3,6-diazabicyclo[3.1.1]heptan-6-yl)-1-oxoisoindolin-2-yl)dihydropyrimidine-2,4 (1H,3H)-dione;
1-(5-((1R,4R)-2,5-diazabicyclo[2.2.1]heptan-2-yl)-1-oxoisoindolin-2-yl)dihydropyrimidine-2,4 (1H,3H)-dione;
1-(5-((1S,4S)-2,5-diazabicyclo[2.2.1]heptan-2-yl)-1-oxoisoindolin-2-yl)dihydropyrimidine-2,4 (1H,3H)-dione;
1-(5-(3,8-diazabicyclo[3.2.1]octan-3-yl)-1-oxoisoindolin-2-yl)dihydropyrimidine-2,4 (1H,3H)-dione;
1-(5-(3,8-diazabicyclo[3.2.1]octan-8-yl)-1-oxoisoindolin-2-yl)dihydropyrimidine-2,4 (1H,3H)-dione;
1-(5-(2,5-diazabicyclo[2.2.2]octan-2-yl)-1-oxoisoindolin-2-yl)dihydropyrimidine-2,4 (1H,3H)-dione;
1-(1-oxo-6-(4-(piperazin-1-yl)piperidin-1-yl)isoindolin-2-yl)dihydropyrimidine-2,4 (1H,3H)-dione;
1-(6-(3,3-difluoro-4-(piperazin-1-yl)piperidin-1-yl)-1-oxoisoindolin-2-yl)dihydropyrimidine-2,4 (1H,3H)-dione;
1-(2-(2,4-dioxotetrahydropyrimidin-1 (2H)-yl)-1-oxoisoindolin-4-yl)piperidine-4-carbaldehyde;
1-(2-(2,4-dioxotetrahydropyrimidin-1 (2H)-yl)-1-oxoisoindolin-5-yl)piperidine-4-carbaldehyde; and
l′-(2-(2,4-dioxotetrahydropyrimidin-1 (2H)-yl)-3-oxoisoindolin-5-yl)-[1,4′-bipiperidine]-4-carbaldehyde.

30. A pharmaceutical composition, comprising:
the compound of Formula (I) or pharmaceutically acceptable salts, enantiomers, stereoisomers, solvates, isotopically enriched analogs, prodrugs, or polymorphs thereof as claimed in claim 1; and
at least one pharmaceutically acceptable carrier;
optionally comprising at least one additional therapeutic agent, e.g., an anticancer agent.

31. (canceled)

32. A medicine kit or reagent kit comprising:
the compound of Formula (I) or a pharmaceutically acceptable salt thereof as claimed in claim 1; or
a pharmaceutical composition comprising the compound of Formula (I) or a pharmaceutically acceptable salt thereof.

33-38. (canceled)

39. A method for treating or preventing a disease or disorder in a subject, comprising administering to the subject a therapeutically effective amount of the compound of Formula (I) or salts, enantiomers, stereoisomers, solvates, isotopically enriched analogs, prodrugs, or polymorphs thereof as claimed in claim 1, or a pharmaceutical composition comprising the compound of Formula (I) or a pharmaceutically acceptable salt thereof, wherein the disease or disorder comprises tumors, autoinflammatory diseases, inflammatory diseases, autoimmune diseases, neurological disorders, respiratory diseases, hemorrhagic shock, transplant rejection, multiple organ dysfunction syndrome (MODS), sarcoidosis, adult respiratory distress syndrome, cardiovascular diseases, Richter syndrome (RS), acute liver failure, diabetes, Kennedy disease, seborrheic alopecia, hirsutism, skin diseases, dysfunctional uterine bleeding, anemia, pediatric aplastic anemia, endometriosis, polycystic ovary syndrome, and thyroid diseases.

40. The method as claimed in claim 39, wherein the disease or disorder is selected from the group consisting of: myeloma, including multiple myeloma, plasma cell myeloma, smoldering myeloma, smoldering multiple myeloma; myelofibrosis; bone marrow disease; myelodysplastic syndrome (MDS); previously treated myelodysplastic syndrome; transplantation-related cancer; neutropenia; leukemia, including chronic myelogenous leukemia, B-cell chronic lymphocytic leukemia, leukemia-associated anemia, acute myeloid leukemia (AML), chronic myeloid leukemia (CML), monocytic leukemia, myelomonocytic leukemia, acute lymphoblastic leukemia (ALL), acute T-lymphocytic leukemia, T-lymphocytic leukemia, chronic lymphocytic leukemia; lymphoma, including diffuse large B-cell lymphoma, non-Hodgkin's lymphoma, Hodgkin's lymphoma, anaplastic lymphoma, anaplastic large cell lymphoma, immunoblastic T-Cell lymphoma, CD20 positive lymphoma, mantle cell lymphoma, follicular lymphoma (FL), Burkitt's lymphoma, marginal zone lymphoma (MZL), primary lymphoma, B-cell lymphoma, recurrent B-cell non-Hodgkin's lymphoma, recurrent diffuse large B-cell lymphoma, recurrent mediastinal (thymic) large B-cell lymphoma, primary mediastinal (thymic) large B-cell lymphoma, recurrent transformed non-Hodgkin's lymphoma, refractory B-cell non-Hodgkin's lymphoma, refractory diffuse large B-cell lymphoma, refractory primary mediastinal (thymic) large B-cell lymphoma, refractory transformed non-Hodgkin's lymphoma; thyroid cancer; melanoma; lung cancer, including lung adenocarcinoma, lung squamous cell carcinoma, non-small cell lung cancer and small cell lung cancer; inflammatory myofibroblastoma; colorectal cancer; intestinal cancer; brain glioma; astroblastoma; glioma; peripheral neuroepithelioma; ovarian cancer; bronchial cancer; prostate cancer; breast cancer, including triple negative breast cancer, sporadic breast cancer, ductal carcinoma of the breast, and patients with Cowden syndrome; pancreatic cancer; central nervous system tumor; neuroblastoma; extramedullary plasmacytoma; plasmacytoma; gastric cancer; gastrointestinal stromal tumors; esophageal cancer; colorectal adenocarcinoma; esophageal squamous cell carcinoma; liver cancer; renal cell carcinoma; bladder cancer; endometrial cancer; metrocarcinoma; head and neck cancer; brain cancer; oral cancer; sarcoma, including rhabdomyosarcoma, various lipogenic tumors, Ewing's sarcoma/primitive neuroectodermal tumors (Ewing/PNETs), and leiomyosarcoma; urothelial carcinoma; basal cell carcinoma; oral squamous cell carcinoma; cholangiocarcinoma; bone cancer; cervical cancer; skin cancer; Unverricht's syndrome; Richter syndrome (RS); sepsis syndrome; autoimmune diseases, including rheumatoid arthritis, autoimmune encephalomyelitis, ankylosing spondylitis, psoriasis, systemic lupus erythematosus, multiple sclerosis, recurrent oral ulcers, Kawasaki disease, polymyositis/dermatomyositis, Sjogren's syndrome, and atopic dermatitis; keratoconjunctivitis; autoinflammatory diseases, including gout, etc; inflammatory diseases, including Crohn's disease and ulcerative colitis, pneumonia, osteoarthritis, synovitis, systemic inflammatory response syndrome, airway inflammation, bronchitis; cerebral malaria; infectious diseases, including viral pneumonia, Acquired immunodeficiency syndrome (AIDS), COVID-19 novel coronavirus infection, gram-negative bacteria infection, gram-positive bacteria infection, tuberculosis, etc; septic shock; bacterial meningitis; chronic obstructive pulmonary disease; asthma; hemorrhagic shock; organ (including kidney, heart, lung) or tissue transplantation rejection; diabetes; sarcoidosis; adult respiratory distress syndrome; multiple organ dysfunction caused by cachexia and septic shock; acute liver failure; dysfunctional uterine bleeding; anemia; pediatric aplastic anemia; endometriosis; polycystic ovary syndrome; thyroid disease; cardiovascular diseases (e.g., coronary heart disease, congestive heart failure, myocardial infarction, atherosclerosis); skin diseases (e.g., acne); Kennedy disease; seborrheic alopecia; and hirsutism.

41. (canceled)

42. A method for treating or preventing a disease or disorder associated with a cereblon protein in a subject, comprising administering to the subject a therapeutically effective amount of the compound of Formula (II) or salts, enantiomers, stereoisomers, solvates, isotopically enriched analogs, prodrugs, or polymorphs thereof as claimed in claim 21, or a pharmaceutical composition comprising the compound of Formula (I) or a pharmaceutically acceptable salt thereof.

43. The method as claimed in claim 42, wherein the disease or disorder associated with cereblon protein comprises tumors, autoinflammatory diseases, inflammatory diseases, autoimmune diseases, neurological disorders, respiratory diseases, hemorrhagic shock, transplant rejection, multiple organ dysfunction syndrome (MODS), sarcoidosis, adult respiratory distress syndrome, cardiovascular diseases, Richter syndrome (RS), acute liver failure, diabetes, Kennedy disease, seborrheic alopecia, hirsutism, skin diseases, dysfunctional uterine bleeding, anemia, pediatric aplastic anemia, endometriosis, polycystic ovary syndrome, and thyroid diseases.

44. The method as claimed in claim 42, wherein the disease or disorder associated with cereblon protein is selected from the group consisting of: myeloma, including multiple myeloma, plasma cell myeloma, smoldering myeloma, smoldering multiple myeloma; myelofibrosis; bone marrow disease; myelodysplastic syndrome (MDS); previously treated myelodysplastic syndrome; transplantation-related cancer; neutropenia; leukemia, including chronic myelogenous leukemia, B-cell chronic lymphocytic leukemia, leukemia-associated anemia, acute myeloid leukemia (AML), chronic myeloid leukemia (CML), monocytic leukemia, myelomonocytic leukemia, acute lymphoblastic leukemia (ALL), acute T-lymphocytic leukemia, T-lymphocytic leukemia, chronic lymphocytic leukemia; lymphoma, including diffuse large B-cell lymphoma, non-Hodgkin's lymphoma, Hodgkin's lymphoma, anaplastic lymphoma, anaplastic large cell lymphoma, immunoblastic T-Cell lymphoma, CD20 positive lymphoma, mantle cell lymphoma, follicular lymphoma (FL), Burkitt's lymphoma, marginal zone lymphoma (MZL), primary lymphoma, B-cell lymphoma, recurrent B-cell non-Hodgkin's lymphoma, recurrent diffuse large B-cell lymphoma, recurrent mediastinal (thymic) large B-cell lymphoma, primary mediastinal (thymic) large B-cell lymphoma, recurrent transformed non-Hodgkin's lymphoma, refractory B-cell non-Hodgkin's lymphoma, refractory diffuse large B-cell lymphoma, refractory primary mediastinal (thymic) large B-cell lymphoma, refractory transformed non-Hodgkin's lymphoma; thyroid cancer; melanoma; lung cancer, including lung adenocarcinoma, lung squamous cell carcinoma, non-small cell lung cancer and small cell lung cancer; inflammatory myofibroblastoma; colorectal cancer; intestinal cancer; brain glioma; astroblastoma; glioma; peripheral neuroepithelioma; ovarian cancer; bronchial cancer; prostate cancer; breast cancer, including triple negative breast cancer, sporadic breast cancer, ductal carcinoma of the breast, and patients with Cowden syndrome; pancreatic cancer; central nervous system tumor; neuroblastoma; extramedullary plasmacytoma; plasmacytoma; gastric cancer; gastrointestinal stromal tumors; esophageal cancer; colorectal adenocarcinoma; esophageal squamous cell carcinoma; liver cancer; renal cell carcinoma; bladder cancer; endometrial cancer; metrocarcinoma; head and neck cancer; brain cancer; oral cancer; sarcoma, including rhabdomyosarcoma, various lipogenic tumors, Ewing's sarcoma/primitive neuroectodermal tumors (Ewing/PNETs), and leiomyosarcoma; urothelial carcinoma; basal cell carcinoma; oral squamous cell carcinoma; cholangiocarcinoma; bone cancer; cervical cancer; skin cancer; Unverricht's syndrome; Richter syndrome (RS); sepsis syndrome; autoimmune diseases, including rheumatoid arthritis, autoimmune encephalomyelitis, ankylosing spondylitis, psoriasis, systemic lupus erythematosus, multiple sclerosis, recurrent oral ulcers, Kawasaki disease, polymyositis/dermatomyositis, Sjogren's syndrome, and atopic dermatitis; keratoconjunctivitis; inflammatory diseases, including Crohn's disease and ulcerative colitis, pneumonia, osteoarthritis, synovitis, systemic inflammatory response syndrome, airway inflammation, bronchitis; cerebral malaria; infectious diseases, including viral pneumonia, Acquired immunodeficiency syndrome (AIDS), COVID-19 novel coronavirus infection, gram-negative bacteria infection, gram-positive bacteria infection, tuberculosis, etc; septic shock; tuberculosis; bacterial meningitis; chronic obstructive pulmonary disease; asthma; hemorrhagic shock; organ (including kidney, heart, lung) or tissue transplantation rejection; diabetes; sarcoidosis; adult respiratory distress syndrome; anemia; pediatric aplastic anemia; cardiovascular diseases (e.g., coronary heart disease, congestive heart failure, myocardial infarction, atherosclerosis); multiple organ dysfunction caused by cachexia and septic shock; and acute liver failure.

45. The compound of Formula (II) or salts, enantiomers, stereoisomers, solvates, isotopically enriched analogs, prodrugs, or polymorphs thereof as claimed in claim 21 for use as a Cereblon protein (CRBN) binder.

46. A method, comprising: preparing the compound of Formula (I) or salts, enantiomers, stereoisomers, solvates, isotopically enriched analogs, prodrugs, or polymorphs thereof as claimed in claim 1 from a compound of Formula (II), 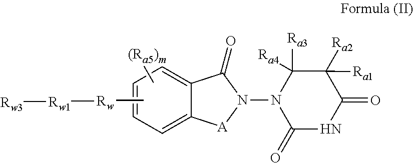
or salts, enantiomers, stereoisomers, solvates, isotopically enriched analogs, prodrugs, or polymorphs thereof,
wherein
A represents CO, CH₂, or CD₂;
Rₐ₁, Rₐ₂, Rₐ₃, and Rₐ₄ are the same or different and independently represent hydrogen, deuterium, halogen, C₁₋₆ alkyl, halogenated C₁₋₆ alkyl, deuterated C₁₋₆ alkyl, C₁₋₆ alkoxy, deuterated C₁₋₆ alkoxy, or halogenated C₁₋₆ alkoxy:
(Rₐ₅)ₘ indicates that the isoindoline ring to which it is attached is optionally substituted with m Rₐ₅, with each Rₐ₅ being the same or different and each independently representing halogen, hydroxy, mercapto, nitro, amino, cyano, C₁₋₆ alkyl, halogenated C₁₋₆ alkyl, deuterated C₁₋₆ alkyl, C₁₋₆ alkoxy, deuterated C₁₋₆ alkoxy, halogenated C₁₋₆ alkoxy, C₂₋₆ alkenyl, or C₂₋₆ alkynyl;
m represents an integer of 0, 1, 2, 3, or 4;
R_{w} represents O, S, S(O), S(O)₂, alkenylene, alkynylene, or N(Rₐ₆), wherein Rₐ₆ represents H or C₁₋₃ alkyl, or R_{w} represents a bond, or
R_{w} represents: 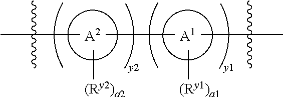
wherein each ring A¹ is the same or different and independently represents nitrogen-containing heterocyclylene, arylene, or heteroarylene, y1 represents an integer of 1 or 2, and (R^{y1})ₐ₁ indicates that each ring A¹ is independently optionally substituted with a1 R^{y1} substituents, wherein each R^{y1} independently represents deuterium, C₁₋₆ alkyl, deuterated C₁₋₆ alkyl, halogenated C₁₋₆ alkyl, C₁₋₆ alkoxy, halogen, amino, hydroxy, mercapto, cyano, carboxyl, C₂₋₆ alkynyl, or C₂₋₆ alkenyl, and a1 represents an integer of 0-20;
each ring A² is the same or different and independently represents heterocyclylene, cycloalkylene, arylene, or heteroarylene, y2 represents an integer of 0 or 1, and (R^{y2})ₐ₂ indicates that each ring A² is independently optionally substituted with a2 R^{y2} substituents, wherein each R^{y2} independently represents deuterium, C₁₋₆ alkyl, deuterated C₁₋₆ alkyl, halogenated C₁₋₆ alkyl, C₁₋₆ alkoxy, halogen, amino, hydroxy, mercapto, cyano, carboxyl, C₂₋₆ alkynyl, or C₂₋₆ alkenyl, and a2 represents an integer of 0-20; and
R_{w1} represents a bond or optionally substituted linear or branched C₁₋₂₀ alkylene, wherein any one or more methylene groups in the main carbon chain skeleton of the linear or branched C₁₋₂₀ alkylene are optionally replaced by one or more groups selected from Rₐ, R_{b}, and any combination thereof; wherein each Rₐ is independently selected from the group consisting of: O, C(O), OC(O), S, S(O), S(O)₂, S(O), N(Rₐ₇), N(Rₐ₇) S(O)₂, C(O)N(Rₐ₇), N(Rₐ₇)C(O), N(Rₐ₇), and N(Rₐ₇)C(O)N(Rₐ₇), where each Rₐ₇ is independently H or C₁₋₃ alkyl, and when one or more adjacent methylene groups in the main carbon chain skeleton of the linear or branched C₁₋₂₀ alkylene are replaced by one or more Rₐ groups, the Rₐ groups are not directly connected to each other; and wherein each R^{b} is independently selected from the group consisting of: optionally substituted cycloalkylene, optionally substituted arylene, optionally substituted heterocyclylene, optionally substituted heteroarylene, alkynylene, alkenylene, and any combination thereof, and
R_{w3} represents hydrogen, deuterium, leaving group, hydroxy, halogen, COOH, NH₂, N₃, CHO, methanesulfonyloxy, trifluoromethanesulfonyloxy or p-toluenesulfonyloxy.

47. A pharmaceutical composition, comprising:
the compound of Formula (II) or pharmaceutically acceptable salts, enantiomers, stereoisomers, solvates, isotopically enriched analogs, prodrugs, or polymorphs thereof as claimed in claim 21; and
at least one pharmaceutically acceptable carrier;
optionally comprising at least one additional therapeutic agent, e.g., an anticancer agent.

48. A medicine kit or reagent kit comprising:
the compound of Formula (II) or a pharmaceutically acceptable salt thereof as claimed in claim 21; or
a pharmaceutical composition comprising the compound of Formula (II) or a pharmaceutically acceptable salt thereof.
